(19) United States
(12) Patent Application Publication
(10) Pub. No.: US 2024/0228488 A1
(43) Pub. Date: Jul. 11, 2024

(54) HETEROARYL INHIBITORS OF PLASMA KALLIKREIN

(71) Applicant: TAKEDA PHARMACEUTICAL COMPANY LIMITED, Osaka-shi Osaka (JP)

(72) Inventors: Nikolaos PAPAIOANNOU; Jeremy Mark TRAVINS, Lexington, MA (US); Sarah Jocelyn FINK; John Mark ELLARD, Saffron Walden (GB); Alastair RAE, Saffron Walden (GB); Jonathan Andrew SPENCER, Saffron Walden (GB); Stuart Shane RANKIN, Saffron Walden (GB); Robert Staurt Laurie CHAPMAN, Saffron Walden (GB)

(21) Appl. No.: 18/550,306

(22) PCT Filed: Mar. 16, 2022
(86) PCT No.: PCT/US2022/020482

§ 371 Date: Sep. 13, 2023

Related U.S. Application Data
(60) Provisional application No. 63/162,468, filed on Mar. 17, 2021.

(51) Int. Cl. C07D 471/04; C07D 519/00

(52) U.S. Cl. CPC C07D 471/04; C07D 519/00

(57) ABSTRACT

The present invention provides compounds and compositions thereof which are useful as inhibitors of plasma kallikrein and which exhibit desirable characteristics for the same.

### CROSS REFERENCE TO RELATED APPLICATIONS

This application claims the benefit of U.S. Provisional Application No. 63/162,468, filed Mar. 17, 2021, which is herein incorporated by reference in its entirety.

### I. BACKGROUND OF THE INVENTION

Plasma Kallikrein (PKa) is a serine protease zymogen in blood that is converted to its catalytically active form by coagulation factor XIIa, and contributes to the innate inflammatory response and intrinsic cascade of blood coagulation. The mechanisms that lead to the activation of this pathway in vivo include interactions with polyphosphates released from activated platelets and deficiency of C1 inhibitor (C1-INH), the primary physiological inhibitor of PKa. PKa-mediated cleavage of high-molecular weight kininogen generates the potent vasodilator and pro-inflammatory nonapeptide bradykinin (BK), which activates the bradykinin 2 receptor. Subsequent cleavage of BK by carboxypeptidases generates des-Arg9-BK, which activates the B1 receptor. Both B1 and B2 receptors are expressed by vascular, glial, and neuronal cell types, with the highest levels of retinal expression detected in the ganglion cell layer and inner and outer nuclear layers. Activation of B1 and B2 receptors causes vasodilation and increases vascular permeability.

PKa is also associated with a number of disorders, such as hereditary angioedema (HAE), an autosomal dominant disease characterized by painful, unpredictable, recurrent attacks of inflammation affecting the hands, feet, face, abdomen, urogenital tract, and the larynx. Prevalence for HAE is uncertain but is estimated to be approximately 1 case per 50,000 persons without known differences among ethnic groups. HAE is caused by deficient (Type I) or dysfunctional (Type II) levels of C1-INH, which inhibits PKa, bradykinin, and other serine proteases in the blood. Individuals with hereditary angioedema (HAE) are deficient in C1-INH and consequently undergo excessive bradykinin generation, which in turn cause painful, debilitating, and potentially fatal swelling attacks. If left untreated, HAE can result in a mortality rate as high as 40% primarily due to upper airway obstruction.

### II. SUMMARY OF THE INVENTION

The present disclosure is based on, at least in part, the development of a number of compounds which bind to plasma kallikrein and effectively inhibit its activity. Accordingly, provided herein are compounds and uses thereof for targeting plasma kallikrein and/or treating plasma kallikrein-mediated diseases and disorders.

In some embodiments, the present invention provides a compound of Formula (I):

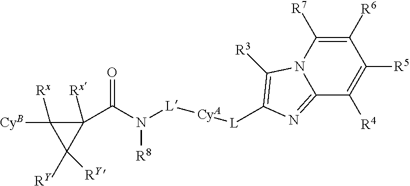

or a pharmaceutically acceptable salt thereof, wherein each of Cy^{A}, Cy^{B}, L, L′, R^{x}, R^{x′}, R^{Y}, R^{Y′}, R³, R⁴, R⁵, R⁶, R⁷, and R⁸ is defined and described in classes and subclasses herein, both singly and in combination. In certain embodiments, the present invention provides compounds of Formulae (I)-(VI-c), as defined and described in classes and subclasses herein. In certain embodiments, the present invention provides novel intermediates and processes for preparing compounds disclosed herein. The disclosure also extends to pharmaceutical compositions comprising any one of the same, and use of compounds or compositions herein for treatment, in particular treatment of autoimmune disease, such as HAE or diabetic macular edema.

In some embodiments, the present invention also provides methods of using compounds of Formulae (I)-(VI-c).

Advantageously, the compounds of the present disclosure have therapeutic activity and/or adequate levels of bioavailability and/or adequate half-life for use as a therapeutic.

### III. DETAILED DESCRIPTION OF CERTAIN EMBODIMENTS

### A. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “halogen” means F, Cl, Br, or I.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

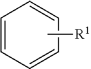

refers to at least

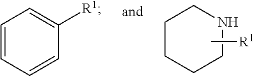

refers to at least

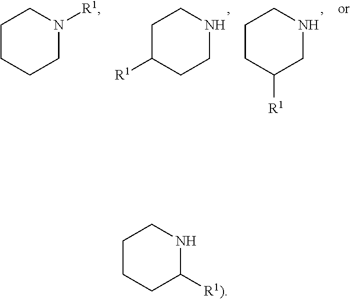

In addition, in a polycyclic ring system, substituents may, unless otherwise indicated, replace a hydrogen on any individual ring (e.g.,

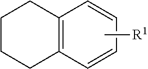

refers to at least

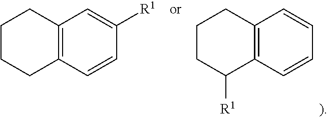

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O(CH₂)₀₋₄C(O)OR^{∘}; —O(CH₂)₀₋₄OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6 membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{#}₂, ═NNHC(O)R^{#}, ═NNHC(O)OR^{#}, ═NNHS(O)₂R^{#}, ═NR^{#}, ═NOR^{#}, —O(C(R^{#}₂))₂₋₃O—, or —S(C(R^{#}₂))₂₋₃S—, wherein each independent occurrence of R^{#} is selected from hydrogen, C₁₋₆ aliphatic which may be substituted as defined below, or an unsubstituted 5-6 membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{#} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%.

The term “oxo,” as used herein, means an oxygen that is double bonded to a carbon atom, thereby forming a carbonyl.

The symbol “ [symbol] ”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, information about a reference compound is historical. In some embodiments, information about a reference compound is stored, for example in a computer-readable medium. In some embodiments, comparison of a particular compound of interest with a reference compound establishes identity with, similarity to, or difference of the particular compound of interest relative to the compound.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### B. Compounds

In some embodiments, a provided compound is of Formula (I):

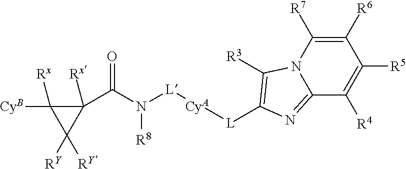

or a pharmaceutically acceptable salt thereof,
wherein:
Cy^{A} is a phenylene or a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 —R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2
heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}—, —S—, —SO—, or SO₂—;
each R³, R⁴, R⁵, R⁶, and R⁷ is independently selected from hydrogen or -L^{C}-R^{C}, wherein
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
      each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups;
         each L^{D} is independently selected from a covalent bond or an optionally substituted C₁₋₆ hydrocarbon chain, wherein 1 to 3 methylene units are optionally and independently replaced with —O— or —NR—;
         each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and
R⁸ is selected from hydrogen, —OR, or an optionally substituted C₁₋₆ aliphatic group.

It will be appreciated that, “oxo” refers a double bonded oxygen substitution on a carbon “C═O”, where the carbon atom is part of the structure or group that is substituted by oxo. For example, where Cy^{C} is substituted with -L^{D}-R^{D}, and where L^{D} is a covalent bond and R^{D} is oxo, the carbon atom substituted with oxo (i.e., the carbon in C═O) is part of Cy^{C} (e.g., a structure of Cy^{C} being cyclopentyl substituted with -L^{D}-R^{D} at the 2-position, where L^{D} is a covalent bond and R^{D} is oxo corresponds to

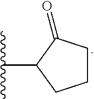 [SMILES: *C1CCCC1=O]

In some embodiments, Cy^{A} is a phenylene or a 5- to 6-membered monocyclic heteroarylene having 1-3 heteroatoms independently selected from oxygen, nitrogen, and sulfur, wherein Cy^{A} is substituted with 0-4 R^{A} groups. In some embodiments, Cy^{A} is a 5- to 6-membered monocyclic heteroarylene having 1-3 heteroatoms independently selected from oxygen, nitrogen, and sulfur, or a 7 to 12-membered bicyclic heteroarylene having 1-4 heteroatoms independently selected from oxygen, nitrogen, and sulfur, wherein Cy^{A} is substituted with 0-4 —R^{A} groups.

In some embodiments, Cy^{A} is a phenylene, wherein Cy^{A} is substituted with 0-4 —R^{A} groups. In some embodiments, Cy^{A} is a phenylene, wherein Cy^{A} is substituted with 0-2 —R^{A} groups.

In some embodiments, Cy^{A} is a 5- to 6-membered monocyclic heteroarylene having 1-3 heteroatoms independently selected from oxygen, nitrogen, and sulfur, wherein Cy^{A} is substituted with 0-4 —R^{A} groups.

In some embodiments, Cy^{A} is a 6-membered monocyclic heteroarylene having 1-3 heteroatoms independently selected from oxygen, nitrogen, and sulfur, wherein Cy^{A} is substituted with 0-4 —R^{A} groups. In some embodiments, Cy^{A} is a 6-membered monocyclic heteroarylene having 1-3 nitrogen heteroatoms, wherein Cy^{A} is substituted with 0-4 R^{A} groups. In some embodiments, Cy^{A} is a pyridinediyl substituted with 0-1 R^{A} groups. In some embodiments, Cy^{A} is a pyrimidinediyl substituted with 0-1 R^{A} groups. In some embodiments, Cy^{A} is a pyridazinediyl substituted with 0-1 R^{A} groups. In some embodiments, Cy^{A} is a triazinediyl substituted with 0-1 R^{A} groups.

In some embodiments, Cy^{A} is a 5-membered monocyclic heteroarylene having 1-3 heteroatoms independently selected from oxygen, nitrogen, and sulfur, wherein Cy^{A} is substituted with 0-2 —R^{A} groups. In some embodiments, Cy^{A} is an unsubstituted thiadiazolediyl. In some embodiments, Cy^{A} is an unsubstituted oxadiazolediyl. In some embodiments, Cy^{A} is an unsubstituted triazolediyl.

In some embodiments, Cy^{A} is a 7- to 12-membered bicyclic heteroarylene having 1-4 heteroatoms independently selected from oxygen, nitrogen, and sulfur, wherein Cy^{A} is substituted with 0-4 —R^{A} groups. In some embodiments, Cy^{A} is a 8- to 12-membered bicyclic heteroarylene having 1-4 heteroatoms independently selected from oxygen, nitrogen, and sulfur, wherein Cy^{A} is substituted with 0-4 —R^{A} groups. In some embodiments, Cy^{A} is a 9-membered bicyclic heteroarylene having 3-4 heteroatoms independently selected from oxygen and nitrogen, wherein Cy^{A} is substituted with 0-1 —R^{A} groups. In some embodiments, Cy^{A} is a 10-membered bicyclic heteroarylene having 3-4 heteroatoms independently selected from oxygen and nitrogen, wherein Cy^{A} is substituted with 0-1 —R^{A} groups.

In some embodiments, Cy^{A} is selected from the group consisting of:

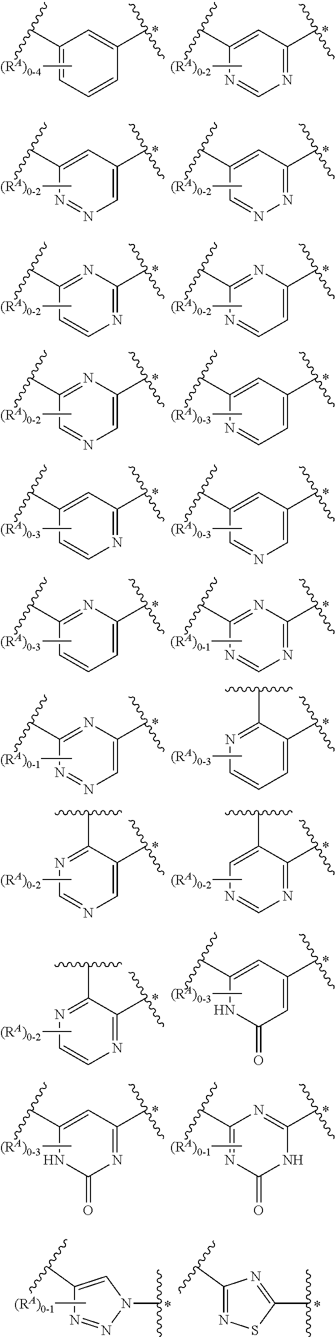

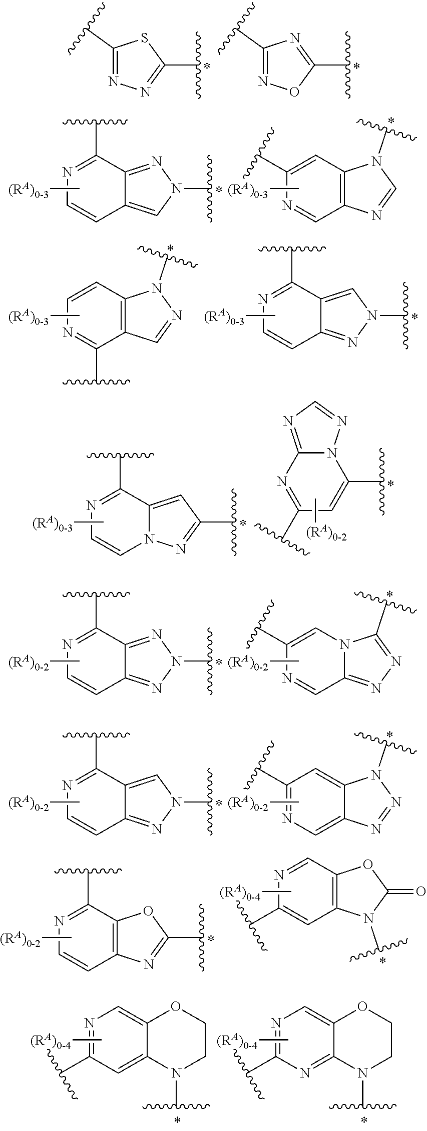

wherein * represents point of attachment to L.

In some embodiments, Cy^{A} is selected from the group consisting of

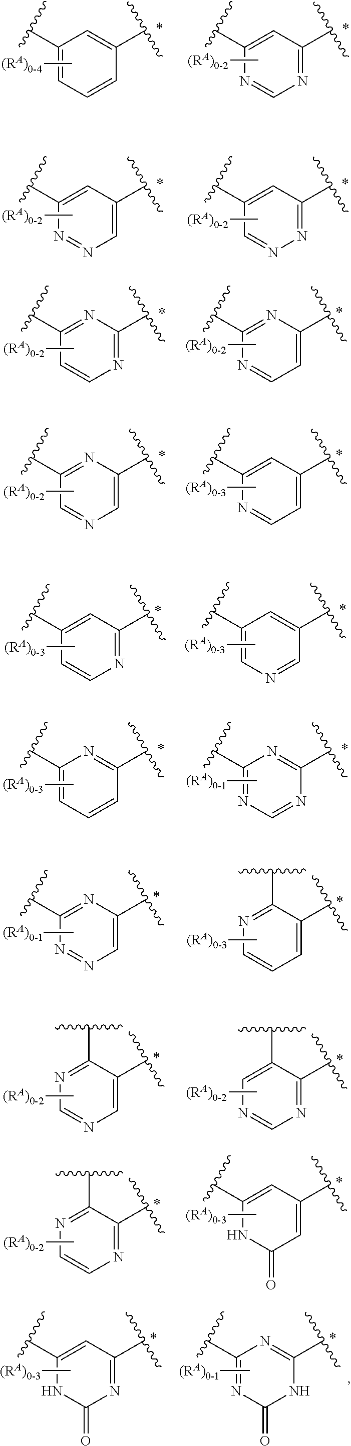

wherein * represents the point of attachment to L.

In some embodiments, Cy^{A} is selected from the group consisting of:

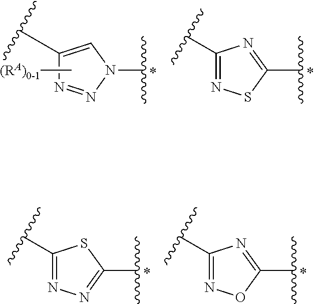

wherein * represents the point of attachment to L.

In some embodiments, Cy^{A} is selected from the group consisting of:

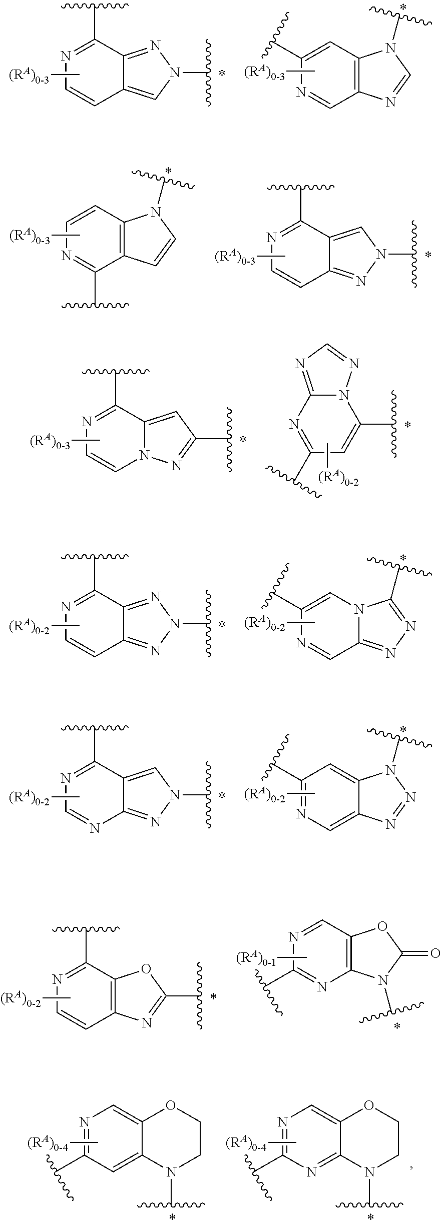

wherein * represents the point of attachment to L.

In some embodiments, Cy^{A} is selected from the group consisting of

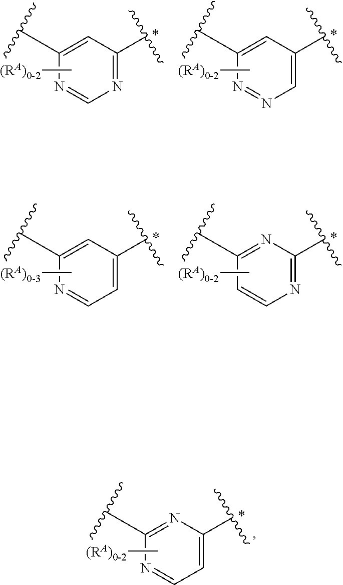

wherein * represents the point of attachment to L.

In some embodiments, Cy^{A} is selected from the group consisting of:

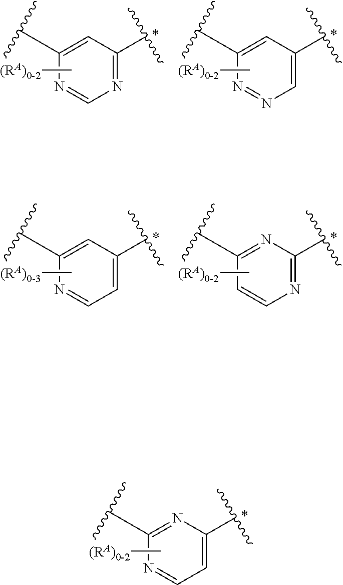

in particular:

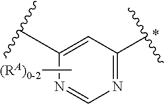

wherein * represents the point of attachment to L.

In some embodiments, each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur.

In some embodiments, Cy^{A} is:

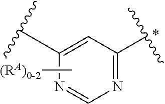

wherein * represents the point of attachment to L.

In some embodiments, Cy^{A} is:

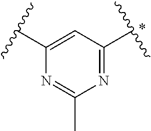 [SMILES: *c1cc(*)nc(C)n1]

wherein * represents the point of attachment to L.

In one embodiment, Cy^{A} comprising 0 R^{A} groups, i.e. Cy^{A} is unsubstituted.

In one embodiment, Cy^{A} comprises 1 R^{A} group, for example as described herein, in particular methyl.

In one embodiment, Cy^{A} comprises 2 R^{A} groups, for example independently selected from the groups/atoms described herein.

In some embodiments, substituents on an optionally substituted R^{A} group are independently halogen, —(CH₂)₀₋₄OR^{∘}, or —(CH₂)₀₋₄N(R^{∘})₂, wherein each R^{∘} is independently as defined above and described in classes and subclasses herein.

In some embodiments, a single instance of R^{A} is oxo. In some embodiments, a single instance of R^{A} is halogen. In some embodiments, a single instance of R^{A} is fluorine. In some embodiments, a single instance of R^{A} is chlorine. In some embodiments, a single instance of R^{A} is —CN. In some embodiments, a single instance of R^{A} is —C(O)₂R. In some embodiments, a single instance of R^{A} is —N(R)₂. In some embodiments, a single instance of R^{A} is —OR. In some embodiments, a single instance of R^{A} is —OMe. In some embodiments, a single instance of R^{A} is —SR. In some embodiments, a single instance of R^{A} is —SR, wherein R is optionally substituted C₁₋₆ aliphatic. In some embodiments, a single instance of R^{A} is —S(O)R. In some embodiments, a single instance of R^{A} is —S(O)R, wherein R is optionally substituted C₁₋₆ aliphatic. In some embodiments, a single instance of R^{A} is —S(O)₂R. In some embodiments, a single instance of R^{A} is —S(O)₂R, wherein R is optionally substituted C₁₋₆ aliphatic. In some embodiments, a single instance of R^{A} is —OR, wherein R is optionally substituted C₁₋₆ aliphatic. In some embodiments, a single instance of R^{A} is —OR, wherein R is C₁₋₆ aliphatic, optionally substituted with —(CH₂)₀₋₄R^{∘}, wherein R^{∘} is phenyl optionally substituted with —OR^{•}, wherein R^{•} is independently as defined above and described in classes and subclasses herein. It will be appreciated that references herein to embodiments in which “a single instance” of a substituent is defined are not limited to monosubstituted embodiments. For example, “[i]n some embodiments, a single instance of R^{A} is oxo” includes embodiments in which at least one instance of R^{A} is oxo and which may comprise one or more additional R^{A} groups as defined herein.

In some embodiments, a single instance of R^{A} is C₁₋₆ aliphatic substituted with halogen. In some embodiments, a single instance of R^{A} is CF₃. In some embodiments, a single instance of R^{A} is C₁₋₆ aliphatic substituted with —(CH₂)₀₋₄OR^{∘}, wherein R^{∘} is selected from hydrogen or C₁₋₆ aliphatic. In some embodiments, a single instance of R^{A} is C₁₋₆ aliphatic substituted with —(CH₂)₀₋₄N(R^{∘})₂, wherein each R^{∘} is independently selected from hydrogen or C₁₋₆ aliphatic.

In some embodiments, a single instance of R^{A} is optionally substituted 3- to 7-membered saturated or partially unsaturated monocyclic carbocyclyl. In some embodiments, a single instance of R^{A} is optionally substituted cyclopropyl.

In some embodiments, a single instance of R^{A} is optionally substituted 3- to 7-membered saturated or partially unsaturated monocyclic heterocyclyl having 1-2 heteroatoms selected from oxygen, nitrogen, or sulfur. In some embodiments, a single instance of R^{A} is optionally substituted 3- to 7-membered saturated monocyclic heterocyclyl having 1-2 heteroatoms selected from oxygen and nitrogen. In some embodiments, a single instance of R^{A} is optionally substituted oxetanyl. In some embodiments, a single instance of R^{A} is oxetanyl optionally substituted with halogen or —(CH₂)₀₋₄OR^{∘}. In some embodiments, a single instance of R^{A} is pyrrolidinyl.

In some embodiments, Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 —R^{B} groups.

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4 —R^{B} groups.

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4 —R^{B} groups, for example pyrimidinyl substituted with 0-4 —R^{B} groups, such as 0 or 1 group (in particular wherein 1 group is methyl).

In some embodiments, Cy^{B} is phenyl, wherein Cy^{B} is substituted with 0-4 —R^{B} groups. In some embodiments, Cy^{B} is phenyl, wherein Cy^{B} is substituted with 0-3 —R^{B} groups.

In some embodiments, Cy^{B} is a 6-membered heteroaryl having 1-3 heteroatoms independently selected from oxygen, nitrogen, and sulfur, wherein Cy^{B} is substituted with 0-4 —R^{B} groups. In some embodiments, Cy^{B} is a 6-membered heteroaryl having 1-3 nitrogens, wherein Cy^{B} is substituted with 0-4 —R^{B} groups. In some embodiments, Cy^{B} is a pyrimidinyl group substituted with 0-2 —R^{B} groups. In some embodiments, Cy^{B} is a pyridinyl group substituted with 0-2 —R^{B} groups. In some embodiments, Cy^{B} is a pyrazinyl group substituted with 0-1 —R^{B} groups. In some embodiments, Cy^{B} is a pyridazinyl group substituted with 0-1 —R^{B} groups. In some embodiments, Cy^{B} is a 1,3,5-triazinyl group substituted with 0-1 —R^{B} groups.

In some embodiments, Cy^{B} is a 5-membered heteroaryl having 1-3 heteroatoms independently selected from oxygen, nitrogen, and sulfur, wherein Cy^{B} is substituted with 0-4 —R^{B}groups. In some embodiments, Cy^{B} is a 5-membered heteroaryl having 1-2 heteroatoms independently selected from sulfur and nitrogen, wherein Cy^{B} is substituted with 0-4 —R^{B} groups. In some embodiments, Cy^{B} is a thienyl group substituted with 0-2 —R^{B} groups. In some embodiments, Cy^{B} is a thiazolyl group substituted with 0-1 —R^{B} groups. In some embodiments, Cy^{B} is a thiadiazolyl group substituted with 0-1 —R^{B} groups.

In some embodiments, Cy^{B} is selected from the group consisting of:

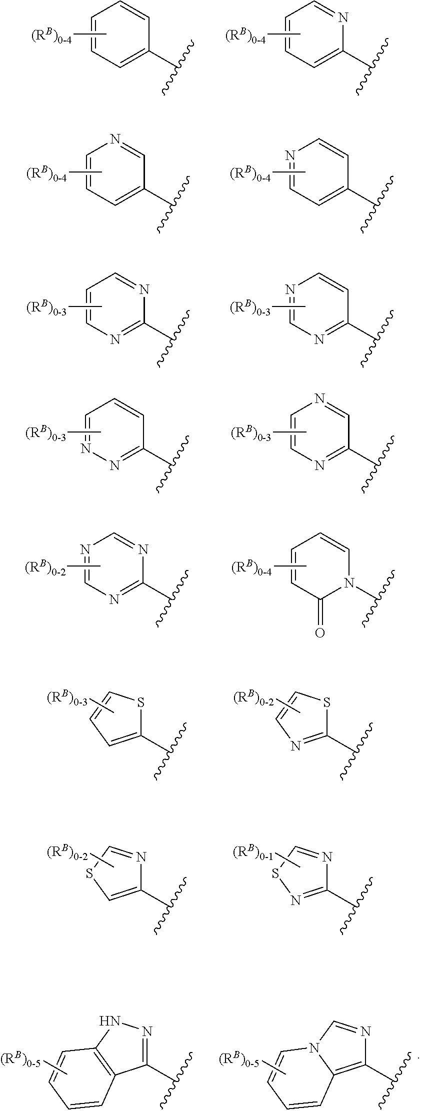

In some embodiments, Cy^{B} is selected from the group consisting of:

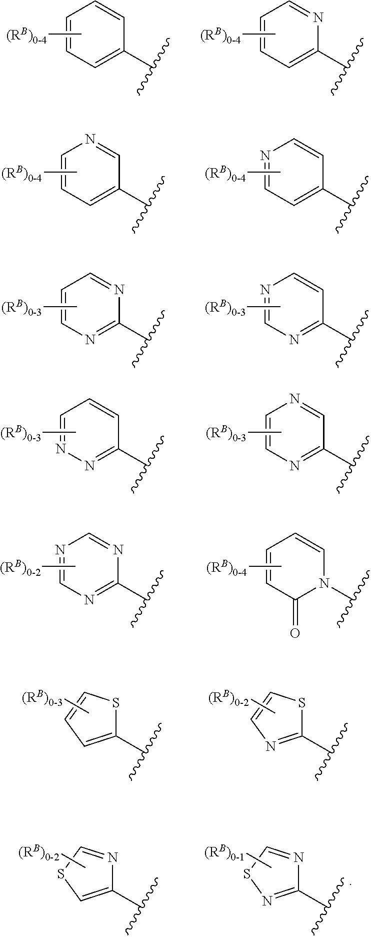

In some embodiments, Cy^{B} is selected from the group consisting of:

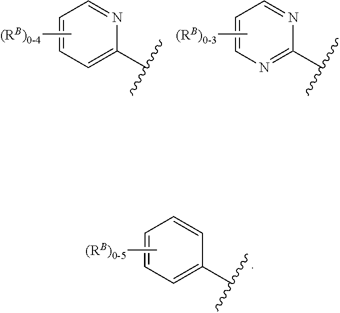

In some embodiments, Cy^{B} is selected from the group consisting of:

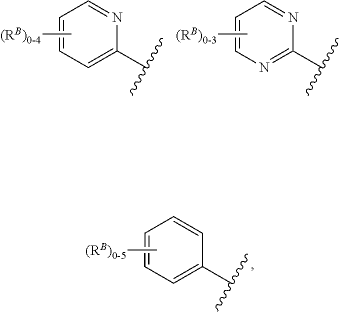

in particular

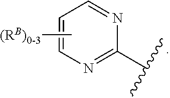

In some embodiments, Cy^{B} is:

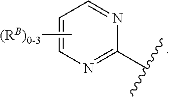

In some embodiments, Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups. It will be appreciated that references herein to the number of atoms in a spirocyclic ring system (e.g., 6- to 12-membered) include the depicted cyclopropyl ring.

In some embodiments, Cy^{B} and R^{x}, together with their intervening atoms, form a 6- to 12-membered spirocyclic ring system having 0-1 nitrogen heteroatoms, wherein the ring or rings formed by Cy^{B} and R^{x} may be substituted with 1-3 —R^{B} groups.

In some embodiments, Cy^{B} and R^{x}, together with their intervening atoms, form a 6- to 12-membered spirocyclic ring system selected from:

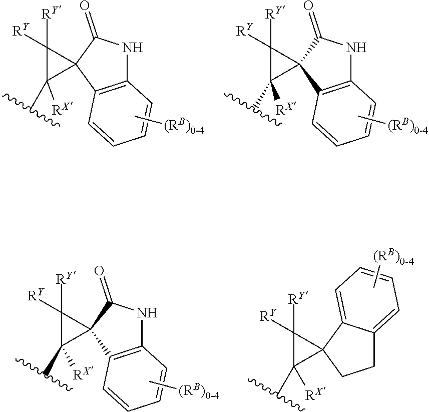

In some embodiments, each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur.

In some embodiments, substituents on an optionally substituted R^{B} group are independently selected from oxo, halogen, and —(CH₂)₀₋₄OR^{∘}, wherein R^{∘} is as defined above and described in classes and subclasses herein.

In some embodiments, a single instance of R^{B} is oxo. In some embodiments, a single instance of R^{B} is halogen. In some embodiments, a single instance of R^{B} is fluorine. In some embodiments, a single instance of R^{B} is chlorine. In some embodiments, a single instance of R^{B} is —CN. In some embodiments, a single instance of R^{B} is —NO₂. In some embodiments, a single instance of R^{B} is —N(R)₂. In some embodiments, a single instance of R^{B} is —NH₂. In some embodiments, a single instance of R^{B} is —N(R)C(O)₂R. In some embodiments, a single instance of R^{B} is —OR. In some embodiments, a single instance of R^{B} is —OMe.

In some embodiments, a single instance of R^{B} is optionally substituted C₁₋₆ aliphatic. In some embodiments, a single instance of R^{B} is C₁₋₆ aliphatic substituted with halogen. In some embodiments, a single instance of R^{B} is methyl. In some embodiments, a single instance of R^{B} is CF₃. In some embodiments, a single instance of R^{B} is CF₂.

In some embodiments, a single instance of R^{B} is —N(R)C(O)₂R, wherein each R is independently selected from hydrogen or C₁₋₆ aliphatic optionally substituted with —(CH₂)₀₋₄R^{∘}.

In some embodiments, a single instance of R^{B} is —OR, wherein each R is independently selected from hydrogen or C₁₋₆ aliphatic optionally substituted with halogen, —(CH₂)₀₋₄OR^{∘}, or (CH₂)₀₋₄C(O)OR^{∘}.

In some embodiments, a single instance of R^{B} is a 5-membered heteroaryl having 1-4 heteroatoms independently selected from oxygen, nitrogen, and sulfur. In some embodiments, a single instance of R^{B} is tetrazolyl.

In some embodiments, each of R^{x} and R^{x′} is independently selected from hydrogen and halogen. In some embodiments R^{x} is H. In some embodiments R^{x′} is H. In some embodiments, each of R^{x} and R^{x′} is hydrogen. In some embodiments, one of R^{x} and R^{x′} is hydrogen and the other is halogen.

In some embodiments, each of R^{Y} and R^{Y′} is independently selected from hydrogen and halogen. In some embodiments R^{Y} is H. Is some embodiments R^{Y′} is H.

In some embodiments, each of R^{Y} and R^{Y′} is hydrogen.

In some embodiments, R^{Y} is an optionally substituted C₁₋₆ aliphatic group.

In some embodiments, R^{Y} is an optionally substituted C₁₋₆ aliphatic group and R^{Y′} is hydrogen. In some embodiments, R^{Y} is substituted with —(CH₂)₀₋₄OR^{∘}, wherein R^{∘} is as defined above and described in classes and subclasses herein.

In some embodiments, L is an optionally substituted C₁₋₃ hydrocarbon chain, wherein 1-3 methylene units are optionally replaced with —O—, —NR^{z}—, —S—, or —SO₂—. In some embodiments, L is an optionally substituted C₁₋₃ hydrocarbon chain, wherein 1 methylene unit is optionally replaced with —O—, —NR^{z}—, —S—, or —SO₂—. In some embodiments, L is an optionally substituted C₁₋₃ hydrocarbon chain, wherein 1 methylene unit is optionally replaced with —O—, —NR^{z}—, —S—, or —SO₂—, in particular —NR^{z}—.

In some embodiments, L is an optionally substituted C₁ hydrocarbon chain.

In some embodiments, L is an optionally substituted C₁ hydrocarbon chain, wherein the 1 methylene unit is replaced with 5-membered saturated or partially unsaturated heterocyclene having 1 nitrogen heteroatom, optionally substituted with —(CH₂)₀₋₄OR^{∘}, wherein R^{∘} is as defined above and described in classes and subclasses herein.

In some embodiments, L is —CH₂—. In some embodiments, L is optionally substituted

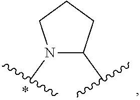 [SMILES: *C1CCCN1*]

wherein * represents the point of attachment to Cy^{A}. In some embodiments, L is optionally substituted

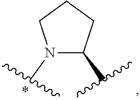 [SMILES: *[C@@H]1CCCN1*]

wherein * represents the point of attachment to Cy^{A}. In some embodiments, L is optionally substituted

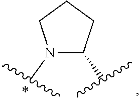 [SMILES: *[C@H]1CCCN1*]

wherein * represents the point of attachment to Cy^{A}. In some embodiments, L is

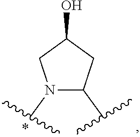 [SMILES: *C1C[C@H](O)CN1*]

wherein * represents the point of attachment to Cy^{A}. In some embodiments, L is

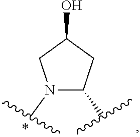 [SMILES: *[C@H]1C[C@H](O)CN1*]

wherein * represents the point of attachment to Cy^{A}. In some embodiments, L is

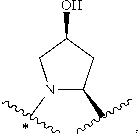 [SMILES: *[C@@H]1C[C@H](O)CN1*]

wherein * represents the point of attachment to Cy^{A}.

In some embodiments, L is an optionally substituted C₂ hydrocarbon chain, wherein 1 methylene unit is optionally replaced with —NR^{z}— or —O—. In some embodiments, L is an optionally substituted C₂ hydrocarbon chain, wherein 1 methylene unit is optionally replaced with —NR^{z}— or —O—, in particular —NR^{z}—. In some embodiments, L is an optionally substituted C₂ hydrocarbon chain, wherein the methylene unit connected to Cy^{A} is replaced with —NR^{z}— or —O—. In some embodiments, L is an optionally substituted C₂ hydrocarbon chain, wherein the methylene unit connected to Cy^{A} is replaced with —NR^{z}—. In some embodiments, L is an optionally substituted C₂ hydrocarbon chain, wherein the methylene unit connected to Cy^{A} is replaced with —NR^{z}—, and wherein R^{z} is selected from hydrogen, —(CH₂)₀₋₃C(O)OR, or an optionally substituted C₁₋₆ aliphatic group. In some embodiments, L is an optionally substituted C₂ hydrocarbon chain, wherein the methylene unit connected to Cy^{A} is replaced with —NR^{z}—, and wherein R^{z} is selected from hydrogen, —(CH₂)₀₋₃C(O)OR, or an optionally substituted C₁₋₆ aliphatic group (such as methyl).

In some embodiments, R^{z} is selected from H and C₁₋₆ aliphatic group, such as H or methyl, in particular methyl.

In some embodiments, L is an optionally substituted C₂ hydrocarbon chain, wherein the methylene unit connected to Cy^{A} is replaced with —O—.

In some embodiments, L is *—NHCH(Me)—, wherein * represents the point of attachment to Cy^{A}. In some embodiments, L is

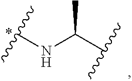 [SMILES: *N[C@H](*)C]

wherein * represents the point of attachment to Cy^{A}. In some embodiments, L is

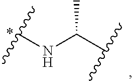 [SMILES: *N[C@@H](*)C]

wherein * represents the point of attachment to Cy^{A}. In some embodiments, L is *—NHCH₂—, wherein * represents the point of attachment to Cy^{A}. In some embodiments, L is *—N(CH₃)CH₂—, wherein * represents the point of attachment to Cy^{A}. In some embodiments, L is

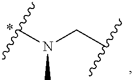

wherein * represents the point of attachment to Cy^{A}. In some embodiments, L is

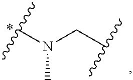

wherein * represents the point of attachment to Cy^{A}. In some embodiments, L is *—OCH(Me)—, wherein * represents the point of attachment to Cy^{A}. In some embodiments, L is *—OCH₂—, wherein * represents the point of attachment to Cy^{A}.

In some embodiments, L comprises a two-atom spacer between Cy^{A} and

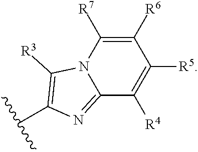

In some embodiments, L is an optionally substituted 5- to 6-membered saturated or partially unsaturated heterocyclene, having 1-3 heteroatoms independently selected from oxygen, nitrogen, and sulfur. In some embodiments, L is an optionally substituted 5-membered saturated or partially unsaturated heterocyclene, having 1 heteroatom independently selected from oxygen, nitrogen, and sulfur. In some embodiments, L is an optionally substituted pyrrolidinediyl group. In some embodiments, L is optionally substituted

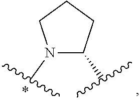 [SMILES: *[C@H]1CCCN1*]

wherein * represents the point of attachment to Cy_{A}.

In some embodiments, optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

In some embodiments, L′ is a covalent bond or a methylene unit optionally substituted with —(CH₂)₀₋₄R^{∘}, wherein R^{∘} is independently as defined above and described in classes and subclasses herein. In some embodiments, R^{∘} is hydrogen or C₁₋₆ aliphatic.

In some embodiments, L′ is a covalent bond.

In some embodiments, each of R³, R⁴, R⁵, R⁶, and R⁷ is independently selected from hydrogen or L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{C}, or an optionally substituted group selected from C₁₋₆ aliphatic.

In some embodiments, R³ is selected from hydrogen or L^{C}-R^{C}, wherein L^{C} is a covalent bond and R^{C} is halogen. In some embodiments, R³ is chloro. In some embodiments, R³ is fluoro. In some embodiments R³ is H.

In some embodiments, R⁴ is selected from hydrogen or L^{C}-R^{C}, wherein L^{C} is selected from a covalent bond or an optionally substituted C₁₋₆ hydrocarbon chain, wherein 1 to 3 methylene units are optionally and independently replaced with —O— or —NR—; and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic. In some embodiments L^{C} is a covalent bond.

In some embodiments, R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic. In some embodiments, R⁴ is chloro. In some embodiments, R⁴ is fluoro.

In some embodiments, R⁴ is selected from the group consisting of:

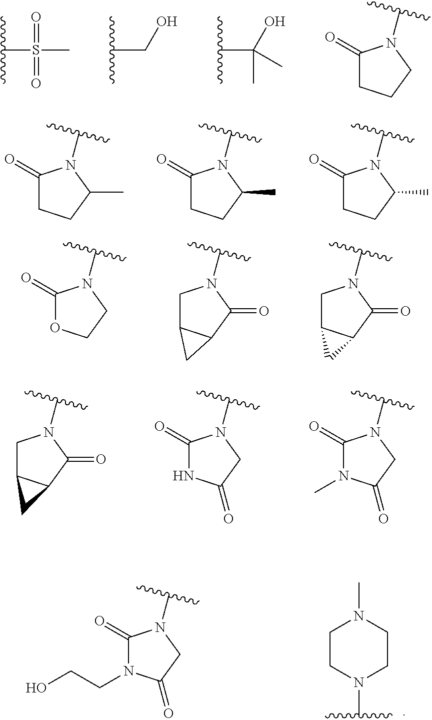

In some embodiments R⁴ is selected from the group consisting of

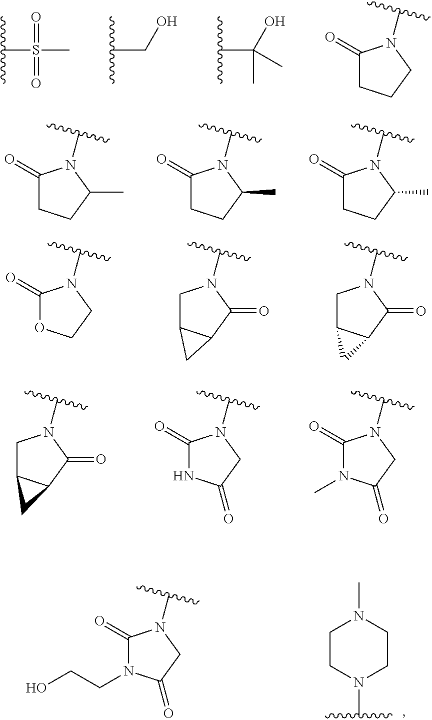

in particular

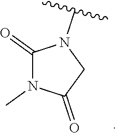 [SMILES: *N1CC(=O)N(C)C1=O]

In some embodiments of R⁴, optional substituents on a C₁₋₆ aliphatic group are selected from —(CH₂)₀₋₄R^{∘}, —(CH₂)₀₋₄OR^{∘}, —CN, —(CH₂)₀₋₄N(R^{∘})₂, and —(CH₂)₀₋₄C(O)OR^{∘}, wherein each R^{∘} is independently as defined above and described in classes and subclasses herein.

In some embodiments of R⁴, Cy^{C} is selected from a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4 -L^{D}-R^{D} groups. In some embodiments of R⁴, Cy^{C} is a 5-membered saturated or partially unsaturated monocyclic heterocyclyl having 1-2 heteroatoms selected from oxygen, nitrogen, or sulfur.

In some embodiments, Cy^{C} is substituted with 0 L^{D}-R^{D} groups. In some embodiments, Cy^{C} is substituted with 1 L^{D}-R^{D} groups. In some embodiments, Cy^{C} is substituted with 2 L^{D}-R^{D} groups. In some embodiments, Cy^{C} is substituted with 3 L^{D}-R^{D} groups. In some embodiments, Cy^{C} is substituted with 4 L^{D}-R^{D} groups.

In some embodiments L^{D} is a covalent bond. In some embodiments R^{D} is optionally substituted C₁₋₆ aliphatic, such as methyl.

In some embodiments of R⁴, Cy^{C} is selected from the group consisting of:

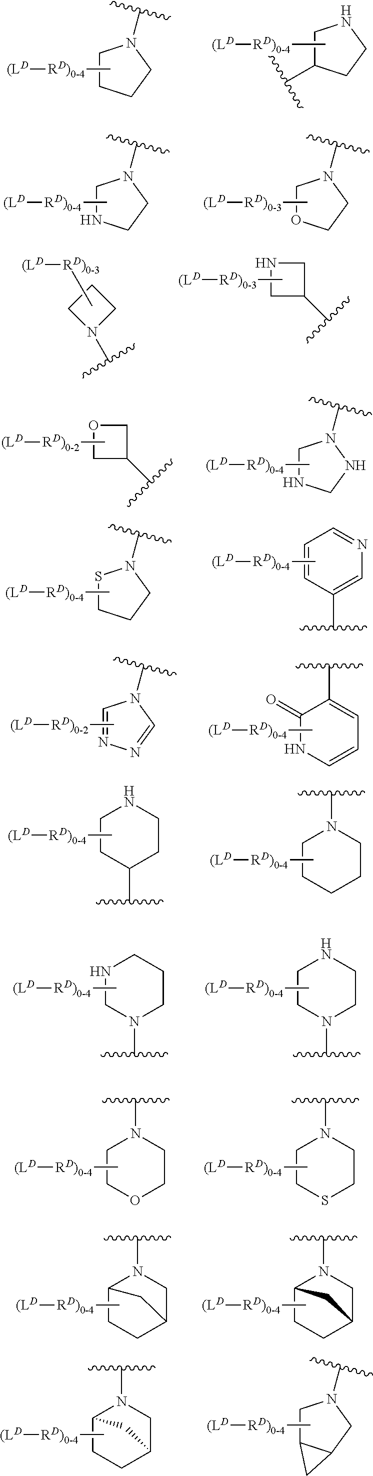

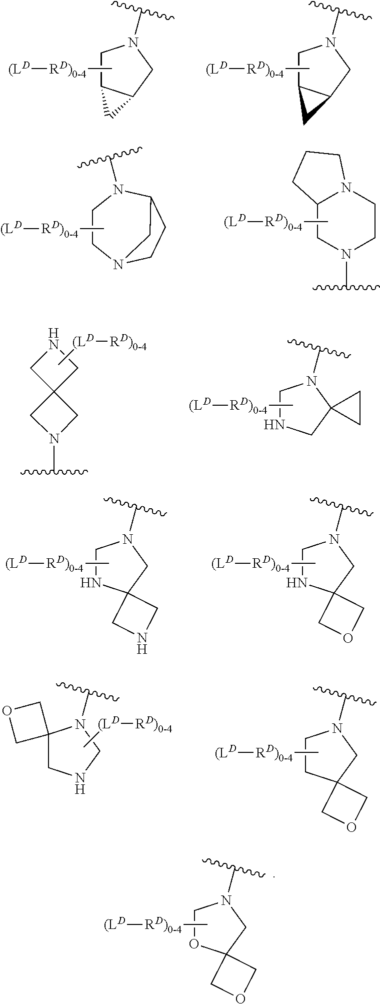

In some embodiments of R⁴, R^{D} is selected from oxo, halogen, —C(O)₂R, —N(R)₂, —OR, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

In some embodiments of a R^{D} group of R⁴, optional substituents on R^{D} are selected from halogen, —(CH₂)₀₋₄R^{∘}, —(CH₂)₀₋₄OR^{∘}, —(CH₂)₀₋₄N(R^{∘})₂, —(CH₂)₀₋₄C(O)OR^{∘}, and —OP(O)(OR^{∘})₂, wherein each R^{∘} is independently as defined above and described in classes and subclasses herein.

In some embodiments of R⁴, L^{D} is a covalent bond.

In some embodiments, R⁵ is hydrogen.

In some embodiments, R⁵ is L^{C}-R^{C}, wherein L^{C} is a covalent bond and R^{C} is Cy^{C}. In some embodiments, Cy^{C} is a cyclopropyl group.

In some embodiments, R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆-aliphatic group.

In some embodiments of R⁶, Cy^{C} is a cyclopropyl group substituted with 0-4 L^{D}-R^{D} groups. In some embodiments of R⁶, Cy^{C} is a cyclopropyl group substituted with methyl or halogen. In some embodiments, L^{D} is a covalent bond and R^{D} is selected from halogen and optionally substituted C₁₋₆ aliphatic.

In some embodiments, R⁷ is selected from hydrogen or L^{C}-R^{C}, wherein L^{C} is a covalent bond, and wherein R^{C} is Cy^{C}.

In some embodiments, R⁷ is hydrogen.

In some embodiments of R⁷, Cy^{C} is:

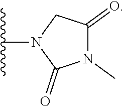 [SMILES: *N1CC(=O)N(C)C1=O]

In some embodiments, R⁸ is hydrogen.

In some embodiments, R⁸ is selected from —OR or an optionally substituted C₁₋₆ aliphatic group.

In some embodiments, a provided compound is of Formula (II):

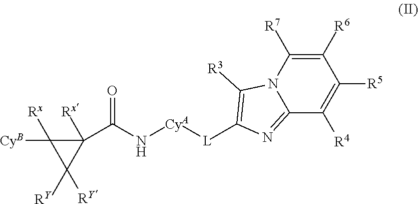

or a pharmaceutically acceptable salt thereof,
wherein each of Cy^{A}, Cy^{B}, L, R^{x}, R^{x′}, R^{Y}, R^{Y′}, R³, R⁴, R⁵, R⁶, and R⁷ is defined and described in classes and subclasses herein, both singly and in combination.

It will be understood that, unless otherwise specified or prohibited by the foregoing definition of Formula (II), embodiments of variables Cy^{A}, Cy^{B}, L, R^{x}, R^{x′}, R^{Y}, R^{Y′}, R³, R⁴, R⁵, R⁶, and R⁷ as defined above and described in classes and subclasses herein, also apply to compounds of Formula (II), both singly and in combination.

In some embodiments, a provided compound is of Formula (III-a), Formula (III-b), or

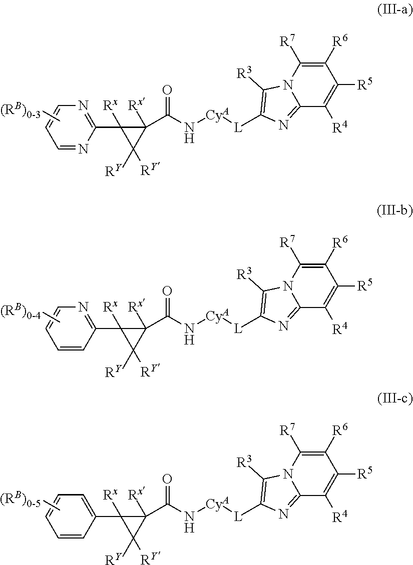

or a pharmaceutically acceptable salt thereof,
wherein each of Cy^{A}, R^{B}, L, R^{x}, R^{x′}, R^{Y}, R^{Y′}, R³, R⁴, R⁵, R⁶, and R⁷ is defined and described in classes and subclasses herein, both singly and in combination, in particular formula (III-a).

It will be understood that, unless otherwise specified or prohibited by the foregoing definitions of Formulae (III-a), (III-b), and (III-c), embodiments of variables Cy^{A}, R^{A}, L, R^{x}, R^{x′}, R^{Y}, R^{Y′}, R³, R⁴, R⁵, R⁶, and R⁷ as defined above and described in classes and subclasses herein, also apply to compounds of Formulae (III-a), (III-b), and (III-c), both singly and in combination.

In some embodiments, a provided compound is of Formula (IV-a), Formula (IV-b), Formula (IV-c), Formula (IV-d), Formula (IV-e), or Formula (IV-e):

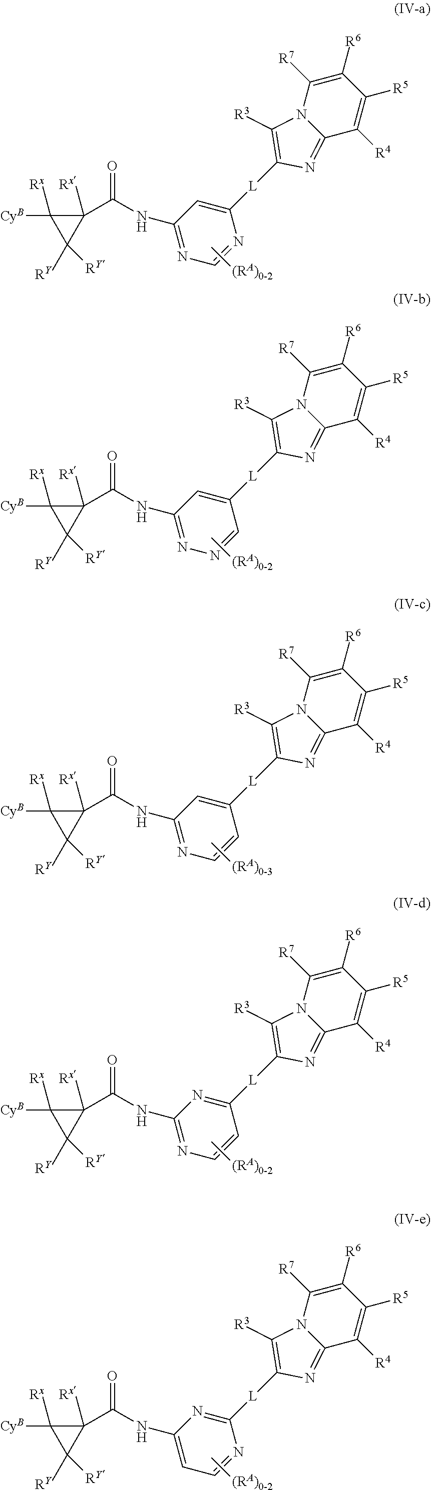

or a pharmaceutically acceptable salt thereof, in particular formula (IV-a).

It will be understood that, unless otherwise specified or prohibited by the foregoing definitions of Formulae (IV-a), (IV-b), (IV-c), (IV-d), and (IV-e), embodiments of variables Cy^{B}, R^{A}, L, R^{x}, R^{x′}, R^{Y}, R^{Y′}, R³, R⁴, R⁵, R⁶, and R⁷ as defined above and described in classes and subclasses herein, also apply to compounds of Formulae (IV-a), (IV-b), (IV-c), (IV-d), and (IV-e), both singly and in combination.

In some embodiments, a provided compound is of Formula (V-a), Formula (V-b), or Formula (V-c):

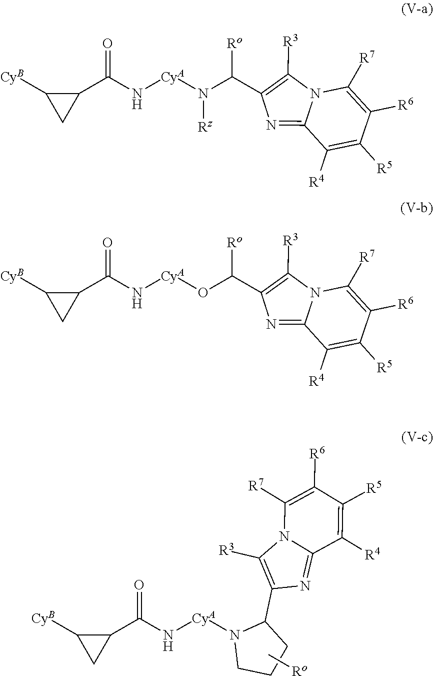

or a pharmaceutically acceptable salt thereof,
wherein each of Cy^{A}, Cy^{B}, R^{z}, R^{∘}, R³, R⁴, R⁵, R⁶, and R⁷ is defined and described in classes and subclasses herein, both singly and in combination, in particular formula (V-a).

It will be understood that, unless otherwise specified or prohibited by the foregoing definitions of Formulae (V-a), (V-b), and (V-c), embodiments of variables Cy^{A}, Cy^{B}, R^{z}, R^{∘}, R³, R⁴, R⁵, R⁶, and R⁷ as defined above and described in classes and subclasses herein, also apply to compounds of Formulae (V-a), (V-b), and (V-c), both singly and in combination.

In some embodiments, a provided compound is of Formula (V-a-1), Formula (V-b-1), or Formula (V-c-1):

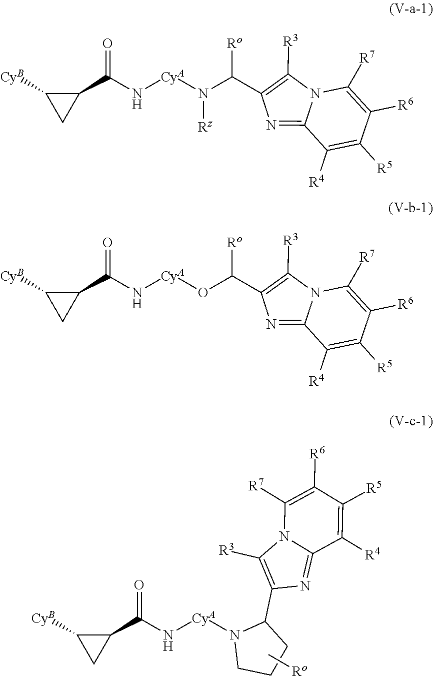

or a pharmaceutically acceptable salt thereof,
wherein each of Cy^{A}, Cy^{B}, R^{z}, R^{∘}, R³, R⁴, R⁵, R⁶, and R⁷ is defined and described in classes and subclasses herein, both singly and in combination, in particular formula (V-a-1).

It will be understood that, unless otherwise specified or prohibited by the foregoing definitions of Formulae (V-a), (V-b), (V-c), (V-a-1), (V-b-1), and (V-c-1), embodiments of variables Cy^{A}, Cy^{B}, R^{z}, R^{∘}, R³, R⁴, R⁵, R⁶, and R⁷ as defined above and described in classes and subclasses herein, also apply to compounds of Formulae (V-a), (V-b), (V-c), (V-a-1), (V-b-1), and (V-c-1), both singly and in combination.

In some embodiments, a provided compound is of Formula (VI-a), Formula (VI-b), or Formula (VI-c):

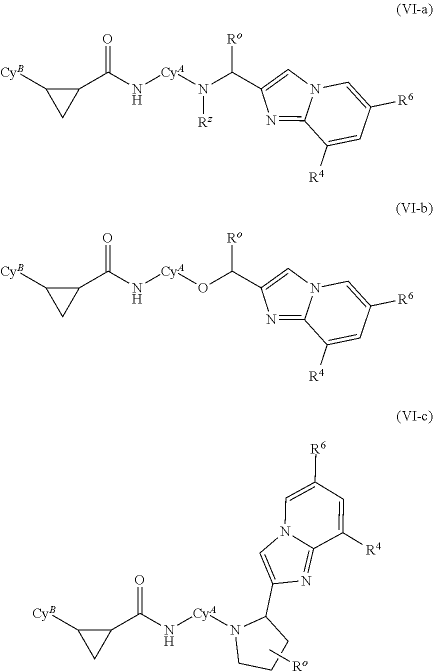

or a pharmaceutically acceptable salt thereof,
wherein each of Cy^{A}, Cy^{B}, R^{z}, and R^{∘} is defined and described in classes and subclasses herein, both singly and in combination;
R⁴ is L^{C}-R^{C}, wherein L^{C} is a covalent bond and R^{C} is Cy^{C}, wherein Cy^{C} is a 5-membered saturated or partially unsaturated monocyclic heterocyclyl having 1-2 heteroatoms selected from nitrogen, and wherein Cy^{C} is substituted with 0-4 -L^{D}-R^{D} groups;
R⁶ is L^{C}-R^{C}, wherein L^{C} is a covalent bond and R^{C} is C₁₋₆ aliphatic or Cy^{C}, wherein Cy^{C} is cyclopropyl optionally substituted with halogen, in particular (VI-a).

It will be understood that, unless otherwise specified or prohibited by the foregoing definitions of Formulae (VI-a), (VI-b), and (VI-c), embodiments of variables Cy^{A}, Cy^{B}, R^{z}, and R^{∘} as defined above and described in classes and subclasses herein, also apply to compounds of Formulae (VI-a), (VI-b), and (VI-c), both singly and in combination.

In some embodiments of Formulae (VI-a), (VI-b), and (VI-c), R⁴ is a ring selected from:

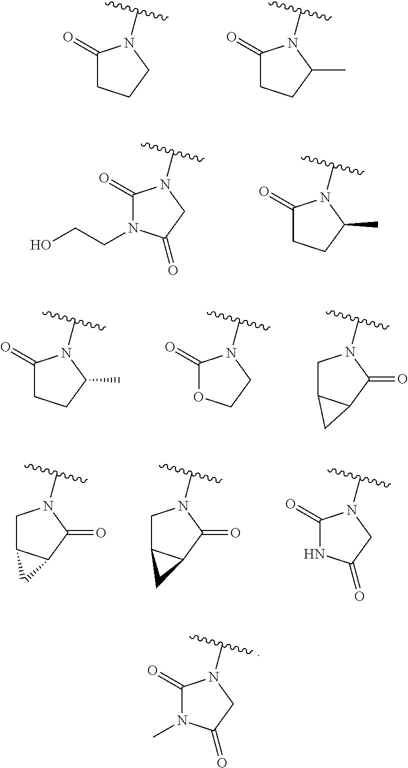

In some embodiments of Formulae (VI-a), (VI-b), and (VI-c), R⁴ is a ring selected from:

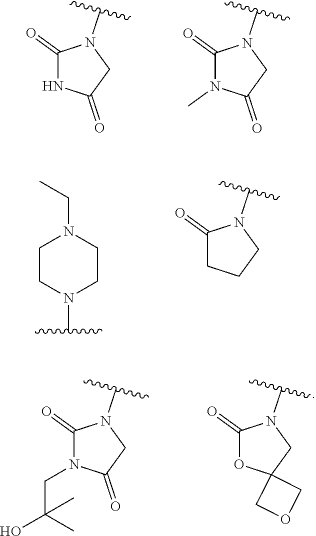

In some embodiments of Formulae (VI-a), (VI-b), and (VI-c) R⁴ is a ring selected from:

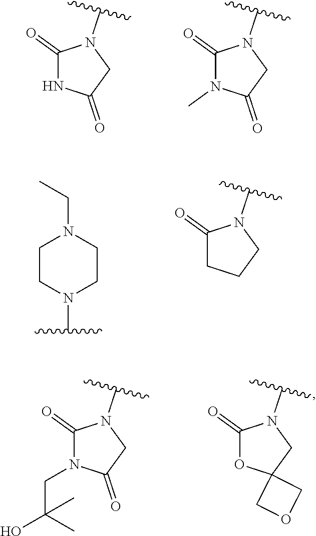

in particular:

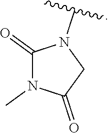

In some embodiments of Formulae (VI-a), (VI-b), and (VI-c), R⁴ is:

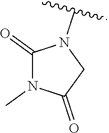

In certain embodiments of provided compounds (i.e., of any species not otherwise defined and of any for Formula (I)-(VI-c), the moiety:

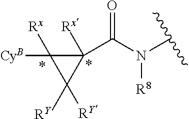

(including where one or more of R^{x}, R^{x′}, R^{Y}, R^{Y′}, or R⁸ is hydrogen) is in the relative trans configuration with respect to the Cy^{B} and amide group attached to the two stereocenters marked with an *. In other words, it will be appreciated that “trans” in the context of the moiety:

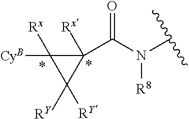

is meant a compound comprising a mixture of:

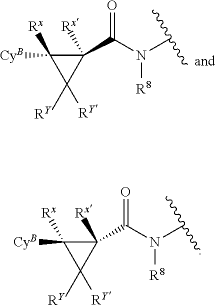

In some embodiments, such a mixture is a racemic mixture.

In certain embodiments of provided compounds (i.e., of any species not otherwise defined and of any of Formula (I)-(VI-c), the absolute stereochemistry of the moiety:

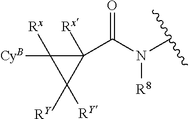

is as follows:

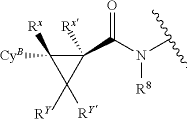

In certain embodiments of provided compounds (i.e., of any species not otherwise defined and of any of Formula (I)-(VI-c), the absolute stereochemistry of the moiety:

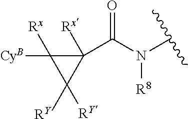

is as follows:

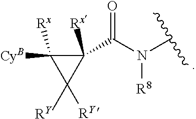

In some embodiments, a provided compound is selected from the group consisting of those in Table A:

### Table A

(1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-1);
(1S,2S)-2-(3-chlorophenyl)-N-(6-(((6-cyclopropyl-8-(4-methylpiperazin-1-yl)imidazo[1,2-a]pyridin-2-yl)methyl)amino)pyrimidin-4-yl)cyclopropane-1-carboxamide (I-2);
(1S,2S)-2-(3-chlorophenyl)-N-(6-(((6-cyclopropyl-8-(4-hydroxypiperidin-1-yl)imidazo[1,2-a]pyridin-2-yl)methyl)amino)pyrimidin-4-yl)cyclopropane-1-carboxamide (I-3);
(1S,2S)-2-(3-chlorophenyl)-N-(4-(((6-cyclopropyl-8-(3-(dimethylamino)azetidin-1-yl)imidazo[1,2-a]pyridin-2-yl)methyl)amino)pyridin-2-yl)cyclopropane-1-carboxamide (I-4);
(1S,2S)-2-(3-chlorophenyl)-N-(6-(((6-cyclopropyl-8-((1-methylpiperidin-4-yl)amino)imidazo[1,2-a]pyridin-2-yl)methyl)amino)pyrimidin-4-yl)cyclopropane-1-carboxamide, formic acid salt (I-5);
(1S,2S)-2-(3-chlorophenyl)-N-(6-(((6-cyclopropyl-8-((1-methylpiperidin-4-yl)amino)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(methoxymethyl)-4-methylpiperazin-1-yl)imidazo[1,2-a]pyridin-2-yl)methyl)amino)pyrimidin-4-yl)cyclopropane-1-carboxamide (I-6);
(1S,2S)-2-(3-chlorophenyl)-N-(6-(((6-cyclopropyl-8-(3-(hydroxymethyl)-4-methylpiperazin-1-yl)imidazo[1,2-a]pyridin-2-yl)methyl)amino)pyrimidin-4-yl)cyclopropane-1-carboxamide, formic acid salt (I-7);
(1S,2S)-2-(3-chlorophenyl)-N-(6-(((6-cyclopropyl-8-(3-(hydroxymethyl)-4-methylpiperazin-1-yl)imidazo[1,2-a]pyridin-2-yl)methyl)amino)pyrimidin-4-yl)cyclopropane-1-carboxamide;
methyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)-1-methylpiperazine-2-carboxylate (I-8);
(1S,2S)-2-(3-chlorophenyl)-N-(6-(((6-cyclopropyl-8-(3-methyl-4-oxoimidazolidin-1-yl)imidazo[1,2-a]pyridin-2-yl)methyl)amino)pyrimidin-4-yl)cyclopropane-1-carboxamide (I-9);
(1S,2S)-2-(3-chlorophenyl)-N-(6-(((6-cyclopropyl-8-(1,1-dioxidothiomorpholino)imidazo[1,2-a]pyridin-2-yl)methyl)amino)pyrimidin-4-yl)cyclopropane-1-carboxamide (I-10);
(1S,2S)—N-(6-(((8-(1,4-diazabicyclo[3.2.1]octan-4-yl)-6-cyclopropylimidazo[1,2-a]pyridin-2-yl)methyl)amino)pyrimidin-4-yl)-2-(3-chlorophenyl)cyclopropane-1-carboxamide (I-11);
(1S,2S)-2-(3-chlorophenyl)-N-(6-(((6-cyclopropyl-8-(6-cyclopropyl-2,6-diazaspiro[3.3]heptan-2-yl)imidazo[1,2-a]pyridin-2-yl)methyl)amino)pyrimidin-4-yl)cyclopropane-1-scarboxamide (I-12);
(1S,2S)-2-(3-chlorophenyl)-N-(6-(((6-cyclopropyl-8-(3-(dimethylamino)pyrrolidin-1-yl)imidazo[1,2-a]pyridin-2-yl)methyl)amino)pyrimidin-4-yl)cyclopropane-1-carboxamide (I-13);
(1S,2S)-2-(3-chlorophenyl)-N-(6-(((6-cyclopropyl-8-(3-(dimethylamino)pyrrolidin-1-yl)imidazo[1,2-a]pyridin-2-yl)methyl)amino)pyrimidin-4-yl)cyclopropane-1-carboxamide, first eluting isomer (I-14);
(1S,2S)-2-(3-chlorophenyl)-N-(6-(((6-cyclopropyl-8-(3-(dimethylamino)pyrrolidin-1-yl)imidazo[1,2-a]pyridin-2-yl)methyl)amino)pyrimidin-4-yl)cyclopropane-1-carboxamide, second eluting isomer (I-15);
(1S,2S)-2-(3-chlorophenyl)-N-(6-(((6-cyclopropyl-8-((R)-3-(dimethylamino)pyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-((S)-3-(dimethylamino)pyrrolidin-1-yl)imidazo[1,2-a]pyridin-2-yl)methyl)amino)pyrimidin-4-yl)cyclopropane-1-carboxamide;
1S,2S)-2-(3-chlorophenyl)-N-(6-(((6-cyclopropyl-8-(3-(methylamino)pyrrolidin-1-yl)imidazo[1,2-a]pyridin-2-yl)methyl)amino)pyrimidin-4-yl)cyclopropane-1-carboxamide (I-16);
(1S,2S)-2-(3-chlorophenyl)-N-(6-(((6-cyclopropyl-8-(3-(dimethylamino)-2-oxopyrrolidin-1-yl)imidazo[1,2-a]pyridin-2-yl)methyl)amino)pyrimidin-4-yl)cyclopropane-1-carboxamide (I-17);
(1S,2S)-2-(3-chlorophenyl)-N-(6-(((6-cyclopropyl-8-(4-(dimethylamino)-2-oxopyrrolidin-1-yl)imidazo[1,2-a]pyridin-2-yl)methyl)amino)pyrimidin-4-yl)cyclopropane-1-carboxamide (I-18);
(1S,2S)-2-(3-chlorophenyl)-N-(6-(((6-cyclopropyl-8-(5-oxoimidazolidin-1-yl)imidazo[1,2-a]pyridin-2-yl)methyl)amino)pyrimidin-4-yl)cyclopropane-1-carboxamide (I-19);
(1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-20);
(1S,2S)-2-(3-chlorophenyl)-N-(4-(((6-cyclopropyl-8-(3-(trifluoromethyl)piperazin-1-yl)imidazo[1,2-a]pyridin-2-yl)methyl)amino)pyridin-2-yl)cyclopropane-1-carboxamide (I-21);
(1S,2S)-2-(3-chlorophenyl)-N-(4-(((6-cyclopropyl-8-(3-(4-methoxybenzyl)-2,4-dioxoimidazolidin-1-yl)imidazo[1,2-a]pyridin-2-yl)methyl)amino)pyridin-2-yl)cyclopropane-1-carboxamide (I-22);
(1S,2S)—N-(4-(((8-(1,4-diazabicyclo[3.2.1]octan-4-yl)-6-cyclopropylimidazo[1,2-a]pyridin-2-yl)methyl)amino)pyridin-2-yl)-2-(3-chlorophenyl)cyclopropane-1-carboxamide (I-23);
(1S,2S)-2-(3-chlorophenyl)-N-(4-(((6-cyclopropyl-8-(6-cyclopropyl-2,6-diazaspiro[3.3]heptan-2-yl)imidazo[1,2-a]pyridin-2-yl)methyl)amino)pyridin-2-yl)cyclopropane-1-carboxamide (I-24);
(1S,2S)-2-(3-chlorophenyl)-N-(4-(((6-cyclopropyl-8-(3-(dimethylamino)pyrrolidin-1-yl)imidazo[1,2-a]pyridin-2-yl)methyl)amino)pyridin-2-yl)cyclopropane-1-carboxamide (I-25);
(1S,2S)-2-(3-chlorophenyl)-N-(4-(((6-cyclopropyl-8-(oxetan-3-ylamino)imidazo[1,2-a]pyridin-2-yl)methyl)amino)pyridin-2-yl)cyclopropane-1-carboxamide (I-26);
(1S,2S)—N-(6-(((R)-1-(6-cyclopropyl-8-(7-oxo-2-oxa-6-azaspiro[3.4]octan-6-yl)imidazo[1,2-a]pyridin-2-yl)ethyl)amino)-2-methylpyrimidin-4-yl)-2-(4-methylpyrimidin-2-yl)cyclopropane-1-carboxamide (I-27);
rac-(1S*,2S*)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-28);
(1R,2R)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-29);
(1S,2S)—N-(6-((6-cyclopropyl-8-(3-methyl-2,4-dioxfoimidazolidin-1-yl)imidazo[1,2-a]pyridin-2-yl)methoxy)pyrimidin-4-yl)-2-(4-methylpyrimidin-2-yl)cyclopropane-1-carboxamide (I-30);
rac-(1S*,2S*)—N-(6-((8-(3-(2-(benzyloxy)ethyl)-2,4-dioxoimidazolidin-1-yl)-6-cyclopropylimidazo[1,2-a]pyridin-2-yl)methoxy)pyrimidin-4-yl)-2-(4-methylpyrimidin-2-yl)cyclopropane-1-carboxamide (I-31);
(1S,2S)—N-(6-((6-cyclopropyl-8-(2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-32);
rac-(1S*,2S*)—N-(6-((6-cyclopropyl-8-(3-(2-hydroxyethyl)-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-33);
(1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide (I-34);
(1S,2S)—N-(5-(1-(6-cyclopropyl-8-(3-methyl-2,4-dioxoimidazolidin-1-yl)imidazo[1,2-a]pyridin-2-yl)ethoxy)-6-methylpyridazin-3-yl)-2-(4-methylpyrimidin-2-yl)cyclopropane-1-carboxamide (I-35);
(1S,2S)—N-(5-(1-(6-methyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-36);
(1S,2S)—N-(5-(1-(6-cyclopropyl-8-(2-oxooxazolidin-3-yl)imidazo[1,2-a]pyridin-2-yl)ethoxy)pyridazin-3-yl)-2-(4-methylpyrimidin-2-yl)cyclopropane-1-carboxamide (I-37);
(1S,2S)—N-(6-((6-cyclopropyl-8-(3-methyl-2,4-dioxoimidazolidin-1-yl)imidazo[1,2-a]pyridin-2-yl)methoxy)-4-methoxypyridin-2-yl)-2-(4-methylpyrimidin-2-yl)cyclopropane-1-carboxamide (I-38);
(1S,2S)—N-(5-(1-(6-cyclopropyl-8-(2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-39);
(1S,2S)—N-(5-(1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-40);
(1S,2S)—N-(5-((S)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-41);
(1S,2S)—N-(5-((R)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-42);
rac-(1S*,2S*)-2-(3-chloro-2-fluorophenyl)-N-(6-(((6-cyclopropylimidazo[1,2-a]pyridin-2-yl)methyl)amino)pyrimidin-4-yl)cyclopropane-1-carboxamide (I-43);
rac-(1S*,2S*)-2-(5-chloro-2-fluorophenyl)-N-(6-(((6-cyclopropylimidazo[1,2-a]pyridin-2-yl)methyl)amino)pyrimidin-4-yl)cyclopropane-1-carboxamide (I-44);
rac-(1S*,2S*)-2-(3-chloro-4-fluorophenyl)-N-(6-(((6-cyclopropylimidazo[1,2-a]pyridin-2-yl)methyl)amino)pyrimidin-4-yl)cyclopropane-1-carboxamide (I-45);
rac-(1S*,2S*)-2-(3-chloro-5-fluorophenyl)-N-(6-(((6-cyclopropylimidazo[1,2-a]pyridin-2-yl)methyl)amino)pyrimidin-4-yl)cyclopropane-1-carboxamide (I-46);
rac-(1S*,2S*)-2-(5-chloro-2-methoxyphenyl)-N-(6-(((6-cyclopropylimidazo[1,2-a]pyridin-2-yl)methyl)amino)pyrimidin-4-yl)cyclopropane-1-carboxamide (I-47);
rac-(1S*,2S*)-2-(5-chloro-2-cyanophenyl)-N-(6-(((6-cyclopropylimidazo[1,2-a]pyridin-2-yl)methyl)amino)pyrimidin-4-yl)cyclopropane-1-carboxamide, formic acid salt (I-48);
rac-(1S*,2S*)-2-(5-chloro-2-cyanophenyl)-N-(6-(((6-cyclopropylimidazo[1,2-a]pyridin-2-yl)methyl)amino)pyrimidin-4-yl)cyclopropane-1-carboxamide;
rac-(1S*,2S*)—N-(6-(((6-cyclopropylimidazo[1,2-a]pyridin-2-yl)methyl)amino)pyrimidin-4-yl)-2-(2,5-dichlorophenyl)cyclopropane-1-carboxamide, formic acid salt (I-49);
rac-(1S*,2S*)—N-(6-(((6-cyclopropylimidazo[1,2-a]pyridin-2-yl)methyl)amino)pyrimidin-4-yl)-2-(2,5-dichlorophenyl)cyclopropane-1-carboxamide;
rac-(1S*,2S*)-2-(3-chloro-6-cyano-2-fluorophenyl)-N-(6-(((6-cyclopropylimidazo[1,2-a]pyridin-2-yl)methyl)amino)pyrimidin-4-yl)cyclopropane-1-carboxamide, formic acid salt (I-50);
rac-(1S*,2S*)-2-(3-chloro-6-cyano-2-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-(difluoromethyl)phenyl)-N-(6-(((6-cyclopropylimidazo[1,2-a]pyridin-2-yl)methyl)amino)pyrimidin-4-yl)cyclopropane-1-carboxamide (I-51);
rac-(1S*,2S*)-2-(5-chloro-2-(trifluoromethyl)phenyl)-N-(6-(((6-cyclopropylimidazo[1,2-a]pyridin-2-yl)methyl)amino)pyrimidin-4-yl)cyclopropane-1-carboxamide (I-52);
rac-(1S*,2S*)—N-(6-(((6-cyclopropylimidazo[1,2-a]pyridin-2-yl)methyl)amino)pyrimidin-4-yl)-2-(3-methoxyphenyl)cyclopropane-1-carboxamide (I-53);
rac-(1S*,2S*)-2-(3-bromophenyl)-N-(6-(((6-cyclopropylimidazo[1,2-a]pyridin-2-yl)methyl)amino)pyrimidin-4-yl)cyclopropane-1-carboxamide (I-54);
rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide (I-55);
(1R,2R)-2-(3-chlorophenyl)-N-(6-(((6-cyclopropylimidazo[1,2-a]pyridin-2-yl)methyl)amino)pyrimidin-4-yl)cyclopropane-1-carboxamide (I-56);
(1S,2S)-2-(3-chlorophenyl)-N-(6-(((6-cyclopropylimidazo[1,2-a]pyridin-2-yl)methyl)amino)pyrimidin-4-yl)cyclopropane-1-carboxamide (I-57);
rac-(1S*,2S*)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide (I-58);
rac-(1S*,2S*)—N-(6-(((8-chloro-6-cyclopropylimidazo[1,2-a]pyridin-2-yl)methyl)amino)pyrimidin-4-yl)-2-(3-chlorophenyl)cyclopropane-1-carboxamide (I-59);
rac-(1S*,2S*)-2-(3-chlorophenyl)-N-(6-(((6-cyclopropyl-8-(methylsulfonyl)imidazo[1,2-a]pyridin-2-yl)methyl)amino)pyrimidin-4-yl)cyclopropane-1-carboxamide (I-60);
rac-ethyl 3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoate (I-61);
(rac-(1S*,2S*))-2-(3-chlorophenyl)-N-(6-(((6-cyclopropyl-8-((S)-4-hydroxy-2-oxopyrrolidin-1-yl)imidazo[1,2-a]pyridin-2-yl)methyl)amino)pyrimidin-4-yl)cyclopropane-1-carboxamide (I-62);
rac-(1S*,2S*)-2-(3-chlorophenyl)-N-(6-(((6-cyclopropyl-8-(3-hydroxyoxetan-3-yl)imidazo[1,2-a]pyridin-2-yl)methyl)amino)pyrimidin-4-yl)cyclopropane-1-carboxamide (I-63);
rac-(1S*,2S*)-2-(3-chlorophenyl)-N-(6-(((6-cyclopropyl-8-(3-fluorooxetan-3-yl)imidazo[1,2-a]pyridin-2-yl)methyl)amino)pyrimidin-4-yl)cyclopropane-1-carboxamide (I-64);
rac-(1S*,2S*)-2-(3-chlorophenyl)-N-(6-(((6-cyclopropyl-8-((R)-4-hydroxy-2-oxopyrrolidin-1-yl)imidazo[1,2-a]pyridin-2-yl)methyl)amino)pyrimidin-4-yl)cyclopropane-1-carboxamide (I-65);
rac-(1S*,2S*)-2-(3-chlorophenyl)-N-(6-(((6-cyclopropyl-8-(4,4-dimethyl-2-oxopyrrolidin-1-yl)imidazo[1,2-a]pyridin-2-yl)methyl)amino)pyrimidin-4-yl)cyclopropane-1-carboxamide (I-66);
rac-(1S*,2S*)-2-(3-chlorophenyl)-N-(6-(((6-cyclopropyl-8-(3-methyl-2-oxopyrrolidin-1-yl)imidazo[1,2-a]pyridin-2-yl)methyl)amino)pyrimidin-4-yl)cyclopropane-1-carboxamide (I-67);
rac-(1S*,2S*)-2-(3-chlorophenyl)-N-(6-(((6-cyclopropyl-8-(2-methyl-3-oxomorpholino)imidazo[1,2-a]pyridin-2-yl)methyl)amino)pyrimidin-4-yl)cyclopropane-1-carboxamide, formic acid salt (I-68);
rac-(1S*,2S*)-2-(3-chlorophenyl)-N-(6-(((6-cyclopropyl-8-(2-methyl-3-oxomorpholino)imidazo[1,2-a]pyridin-2-yl)methyl)amino)pyrimidin-4-yl)cyclopropane-1-carboxamide;
rac-(1S*,2S*)—N-(6-(((3-chloro-6-cyclopropylimidazo[1,2-a]pyridin-2-yl)methyl)amino)pyrimidin-4-yl)-2-(3-chlorophenyl)cyclopropane-1-carboxamide (I-69);
rac-(1S*,2S*)-2-(3-chlorophenyl)-N-(6-(((6-cyclopropyl-8-(hydroxymethyl)imidazo[1,2-a]pyridin-2-yl)methyl)amino)pyrimidin-4-yl)cyclopropane-1-carboxamide, formic acid salt (I-70);
rac-(1S*,2S*)-2-(3-chlorophenyl)-N-(6-(((6-cyclopropyl-8-(hydroxymethyl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((8-cyano-6-cyclopropylimidazo[1,2-a]pyridin-2-yl)methyl)amino)pyrimidin-4-yl)cyclopropane-1-carboxamide (I-71);
rac-3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoic acid (I-72);
rac-(1S*,2S*)-2-(3-chlorophenyl)-N-(6-(((6-cyclopropyl-8-(morpholinomethyl)imidazo[1,2-a]pyridin-2-yl)methyl)amino)pyrimidin-4-yl)cyclopropane-1-carboxamide (I-73);
rac-(1S*,2S*)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide (I-74);
rac-(1S*,2S*)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide, first eluting isomer (I-75);
rac-(1S*,2S*)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide, second eluting isomer (I-76);
(1S,2S)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide;
(1R,2R)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropylimidazo[1,2-a]pyridin-2-yl)methyl)(2-hydroxyethyl)amino)pyrimidin-4-yl)cyclopropane-1-carboxamide (I-77);
rac-(1S*,2S*)-2-(3-chlorophenyl)-N-(4-(((6-cyclopropylimidazo[1,2-a]pyridin-2-yl)methyl)amino)pyrimidin-5-yl)cyclopropane-1-carboxamide (I-78);
rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide (I-79);
rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, first eluting isomer (I-80);
rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, second eluting isomer (I-81);
(1S,2S)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
(1R,2R)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
rac-(1S*,2S*)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-82);
(1R,2R)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-83);
(1S,2S)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-84);
(1S,2S)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide (I-85);
rac-(1S*,2S*)-2-(3-chlorophenyl)-N-(4-(((6-cyclopropylimidazo[1,2-a]pyridin-2-yl)methyl)amino)-5-fluoropyridin-2-yl)cyclopropane-1-carboxamide, formic acid salt (I-86);
rac-(1S*,2S*)-2-(3-chlorophenyl)-N-(4-(((6-cyclopropylimidazo[1,2-a]pyridin-2-yl)methyl)amino)-5-fluoropyridin-2-yl)cyclopropane-1-carboxamide;
rac-(1S*,2S*)-2-(3-chlorophenyl)-N-(4-(((8-(2-cyanoethyl)-6-cyclopropylimidazo[1,2-a]pyridin-2-yl)methyl)amino)pyridin-2-yl)cyclopropane-1-carboxamide (I-87);
(1S,2S)-2-(3-chlorophenyl)-N-(4-(((6-cyclopropyl-8-(4-methylpiperazin-1-yl)imidazo[1,2-a]pyridin-2-yl)methyl)amino)pyrimidin-2-yl)cyclopropane-1-carboxamide (I-88);
(1S,2S)-2-(3-chlorophenyl)-N-(4-(((6-cyclopropyl-8-(2-methyl-5-oxopyrrolidin-1-yl)imidazo[1,2-a]pyridin-2-yl)methyl)amino)pyridin-2-yl)cyclopropane-1-carboxamide (I-89);
(1S,2S)-2-(3-chlorophenyl)-N-(4-(((6-cyclopropyl-8-(2-methyl-5-oxopyrrolidin-1-yl)imidazo[1,2-a]pyridin-2-yl)methyl)amino)pyridin-2-yl)cyclopropane-1-carboxamide, first eluting isomer (I-90);
(1S,2S)-2-(3-chlorophenyl)-N-(4-(((6-cyclopropyl-8-(2-methyl-5-oxopyrrolidin-1-yl)imidazo[1,2-a]pyridin-2-yl)methyl)amino)pyridin-2-yl)cyclopropane-1-carboxamide, second eluting isomer (I-91);
(1S,2S)-2-(3-chlorophenyl)-N-(4-(((6-cyclopropyl-8-((S)-2-methyl-5-oxopyrrolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((R)-2-methyl-5-oxopyrrolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-oxo-2-azabicyclo[2.2.1]heptan-2-yl)imidazo[1,2-a]pyridin-2-yl)methyl)amino)pyridin-2-yl)cyclopropane-1-carboxamide (I-92);
(1S,2S)-2-(3-chlorophenyl)-N-(4-(((6-cyclopropyl-8-(3-oxo-2-azabicyclo[2.2.1]heptan-2-yl)imidazo[1,2-a]pyridin-2-yl)methyl)amino)pyridin-2-yl)cyclopropane-1-carboxamide, first eluting isomer (I-93);
(1S,2S)-2-(3-chlorophenyl)-N-(4-(((6-cyclopropyl-8-(3-oxo-2-azabicyclo[2.2.1]heptan-2-yl)imidazo[1,2-a]pyridin-2-yl)methyl)amino)pyridin-2-yl)cyclopropane-1-carboxamide, second eluting isomer (I-94);
(1S,2S)-2-(3-chlorophenyl)-N-(4-(((6-cyclopropyl-8-((1R,4S)-3-oxo-2-azabicyclo[2.2.1]heptan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1S,4R)-3-oxo-2-azabicyclo[2.2.1]heptan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(2-oxo-3-azabicyclo[3.1.0]hexan-3-yl)imidazo[1,2-a]pyridin-2-yl)methyl)amino)pyridin-2-yl)cyclopropane-1-carboxamide (I-95);
(1S,2S)-2-(3-chlorophenyl)-N-(4-(((6-cyclopropyl-8-(2-oxo-3-azabicyclo[3.1.0]hexan-3-yl)imidazo[1,2-a]pyridin-2-yl)methyl)amino)pyridin-2-yl)cyclopropane-1-carboxamide, first eluting isomer (I-96);
(1S,2S)-2-(3-chlorophenyl)-N-(4-(((6-cyclopropyl-8-(2-oxo-3-azabicyclo[3.1.0]hexan-3-yl)imidazo[1,2-a]pyridin-2-yl)methyl)amino)pyridin-2-yl)cyclopropane-1-carboxamide, second eluting isomer (I-97);
(1S,2S)-2-(3-chlorophenyl)-N-(4-(((6-cyclopropyl-8-((1S,5R)-2-oxo-3-azabicyclo[3.1.0]hexan-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1R,5S)-2-oxo-3-azabicyclo[3.1.0]hexan-3-yl)imidazo[1,2-a]pyridin-2-yl)methyl)amino)pyridin-2-yl)cyclopropane-1-carboxamide;
rac-(1S*,2S*)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-98);
(1S,2S)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-99);
rac-(1S*,2S*)-2-(5-chloro-2-cyanophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-100);
rac-(1S*,2S*)-2-(3-chlorophenyl)-N-(5-(((6-cyclopropylimidazo[1,2-a]pyridin-2-yl)methyl)amino)pyridazin-3-yl)cyclopropane-1-carboxamide (I-101);
rac-(1S*,2S*)-2-(5-chloro-2-cyanophenyl)-N-(5-(((6-cyclopropylimidazo[1,2-a]pyridin-2-yl)methyl)amino)pyridazin-3-yl)cyclopropane-1-carboxamide (I-102);
rac-(1S*,2S*)-2-(3-chlorophenyl)-N-(5-((6-cyclopropylimidazo[1,2-a]pyridin-2-yl)methoxy)pyridazin-3-yl)cyclopropane-1-carboxamide (I-103);
rac-(1S*,2S*)-2-(3-chlorophenyl)-N-(6-((6-cyclopropylimidazo[1,2-a]pyridin-2-yl)methoxy)pyrimidin-4-yl)cyclopropane-1-carboxamide (I-104);
rac-(1S*,2S*)-2-(3-chlorophenyl)-N-(6-(((6-cyclopropylimidazo[1,2-a]pyridin-2-yl)methyl)amino)-2-(pyrrolidin-1-yl)pyrimidin-4-yl)cyclopropane-1-carboxamide (I-105);
rac-(1S*,2S*)-2-(3-chlorophenyl)-N-(6-(((6-cyclopropylimidazo[1,2-a]pyridin-2-yl)methyl)amino)-2-(methylthio)pyrimidin-4-yl)cyclopropane-1-carboxamide (I-106);
rac-(1S*,2S*)-2-(3-chlorophenyl)-N-(6-(((6-cyclopropylimidazo[1,2-a]pyridin-2-yl)methyl)amino)-2-methoxypyrimidin-4-yl)cyclopropane-1-carboxamide (I-107);
rac-(1S*,2S*)-2-(3-chlorophenyl)-N-(6-(((6-cyclopropylimidazo[1,2-a]pyridin-2-yl)methyl)amino)-2-(methoxymethyl)pyrimidin-4-yl)cyclopropane-1-carboxamide (I-108);
rac-(1S*,2S*)-2-(3-chlorophenyl)-N-(6-(((6-cyclopropylimidazo[1,2-a]pyridin-2-yl)methyl)amino)-2-(methylsulfinyl)pyrimidin-4-yl)cyclopropane-1-carboxamide (I-109);
rac-(1S*,2S*)-2-(3-chlorophenyl)-N-(6-(((6-cyclopropylimidazo[1,2-a]pyridin-2-yl)methyl)amino)-2-(methylsulfonyl)pyrimidin-4-yl)cyclopropane-1-carboxamide (I-110);
rac-ethyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate (I-111);
rac-4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylic acid (I-112);
rac-(1S*,2S*)—N-(6-chloro-2-(3-hydroxyoxetan-3-yl)pyrimidin-4-yl)-2-(4-methylpyrimidin-2-yl)cyclopropane-1-carboxamide (I-113);
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-fluorooxetan-3-yl)pyrimidin-4-yl)-2-(4-methylpyrimidin-2-yl)cyclopropane-1-carboxamide (I-114);
rac-(1S*,2S*)-2-(3-chlorophenyl)-N-(4-(((6-cyclopropylimidazo[1,2-a]pyridin-2-yl)methyl)amino)pyridin-2-yl)cyclopropane-1-carboxamide (I-115);
(1R,2R)-2-(3-chlorophenyl)-N-(4-(((6-cyclopropylimidazo[1,2-a]pyridin-2-yl)methyl)amino)pyridin-2-yl)cyclopropane-1-carboxamide (I-116);
(1S,2S)-2-(3-chlorophenyl)-N-(4-(((6-cyclopropylimidazo[1,2-a]pyridin-2-yl)methyl)amino)pyridin-2-yl)cyclopropane-1-carboxamide (I-117);
rac-(1S*,2S*)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide (I-118);
rac-(1S*,2S*)-2-(3-chlorophenyl)-N-(4-(((8-cyano-6-cyclopropylimidazo[1,2-a]pyridin-2-yl)methyl)amino)pyridin-2-yl)cyclopropane-1-carboxamide (I-119);
rac-(1S*,2S*)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (I-120);
(1S,2S)-2-(3-chlorophenyl)-N-(4-(((6-cyclopropylimidazo[1,2-a]pyridin-2-yl)methyl)amino)pyridin-2-yl)cyclopropane-1-carboxamide (I-121);
(1S,2S)-2-(3-chlorophenyl)-N-(4-(((6-cyclopropyl-8-(2-hydroxypropan-2-yl)imidazo[1,2-a]pyridin-2-yl)methyl)amino)pyridin-2-yl)cyclopropane-1-carboxamide (I-122);
(1S,2S)-2-(3-chlorophenyl)-N-(4-(((6-isopropylimidazo[1,2-a]pyridin-2-yl)methyl)amino)pyridin-2-yl)cyclopropane-1-carboxamide (I-123);
(1S,2S)-2-(3-chlorophenyl)-N-(4-(((6-cyclopropyl-8-(3,3-difluoroazetidin-1-yl)imidazo[1,2-a]pyridin-2-yl)methyl)amino)pyridin-2-yl)cyclopropane-1-carboxamide (I-124);
(1S,2S)-2-(3-chlorophenyl)-N-(4-(((6-cyclopropyl-8-(3-fluoroazetidin-1-yl)imidazo[1,2-a]pyridin-2-yl)methyl)amino)pyridin-2-yl)cyclopropane-1-carboxamide (I-125);
(1S,2S)-2-(3-chlorophenyl)-N-(4-(((6-cyclopropyl-8-(methylsulfonamido)imidazo[1,2-a]pyridin-2-yl)methyl)amino)pyridin-2-yl)cyclopropane-1-carboxamide (I-126);
(1S,2S)-2-(3-chlorophenyl)-N-(4-(((6-cyclopropyl-8-(4-methyl-2-oxopiperazin-1-yl)imidazo[1,2-a]pyridin-2-yl)methyl)amino)pyridin-2-yl)cyclopropane-1-carboxamide (I-127);
(1S,2S)-2-(3-chlorophenyl)-N-(4-(((6-cyclopropyl-8-(N-methylmethylsulfonamido)imidazo[1,2-a]pyridin-2-yl)methyl)amino)pyridin-2-yl)cyclopropane-1-carboxamide, formic acid salt (I-128);
(1S,2S)-2-(3-chlorophenyl)-N-(4-(((6-cyclopropyl-8-(N-methylmethylsulfonamido)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-morpholinoimidazo[1,2-a]pyridin-2-yl)methyl)amino)pyridin-2-yl)cyclopropane-1-carboxamide (I-129);
(1S,2S)-2-(3-chlorophenyl)-N-(4-(((6-cyclopropyl-8-(4-methyl-3-oxopiperazin-1-yl)imidazo[1,2-a]pyridin-2-yl)methyl)amino)pyridin-2-yl)cyclopropane-1-carboxamide (I-130);
rac-(1S*,2S*)-2-(5-chloro-2-cyanophenyl)-N-(4-(((6-cyclopropyl-8-(2-hydroxypropan-2-yl)imidazo[1,2-a]pyridin-2-yl)methyl)amino)pyridin-2-yl)cyclopropane-1-carboxamide (I-131);
(1S,2S)-2-(3-chlorophenyl)-N-(4-(((6-cyclopropyl-8-(piperazin-1-yl)imidazo[1,2-a]pyridin-2-yl)methyl)amino)pyridin-2-yl)cyclopropane-1-carboxamide (I-132);
(1S,2S)-2-(3-chlorophenyl)-N-(4-(((6-cyclopropyl-8-(3-fluoroazetidin-3-yl)imidazo[1,2-a]pyridin-2-yl)methyl)amino)pyridin-2-yl)cyclopropane-1-carboxamide, formic acid salt (I-133);
(1S,2S)-2-(3-chlorophenyl)-N-(4-(((6-cyclopropyl-8-(3-fluoroazetidin-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-hydroxyazetidin-3-yl)imidazo[1,2-a]pyridin-2-yl)methyl)amino)pyridin-2-yl)cyclopropane-1-carboxamide (I-134);
(1S,2S)—N-(4-(((8-(-azetidin-3-yl(hydroxy)methyl)-6-cyclopropylimidazo[1,2-a]pyridin-2-yl)methyl)amino)pyridin-2-yl)-2-(3-chlorophenyl)cyclopropane-1-carboxamide (I-135);
(1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide (I-137);
(1S,2S)-2-(3-chlorophenyl)-N-(4-(((6-cyclopropyl-8-(4-methylpiperazin-1-yl)imidazo[1,2-a]pyridin-2-yl)methyl)amino)pyridin-2-yl)cyclopropane-1-carboxamide (I-138);
(1S,2S)-2-(3-chlorophenyl)-N-(4-(((6-cyclopropyl-8-(N-methylacetamido)imidazo[1,2-a]pyridin-2-yl)methyl)amino)pyridin-2-yl)cyclopropane-1-carboxamide (I-139);
(1S,2S)-2-(3-chlorophenyl)-N-(4-(((6-cyclopropyl-8-(6-methyl-2,6-diazaspiro[3.3]heptan-2-yl)imidazo[1,2-a]pyridin-2-yl)methyl)amino)pyridin-2-yl)cyclopropane-1-carboxamide (I-140);
((1S,2S)-2-(3-chlorophenyl)-N-(4-((1-(6-cyclopropylimidazo[1,2-a]pyridin-2-yl)-2,2,2-trifluoroethyl)amino)pyridin-2-yl)cyclopropane-1-carboxamide, formic acid salt (I-141);
((1S,2S)-2-(3-chlorophenyl)-N-(4-((1-(6-cyclopropylimidazo[1,2-a]pyridin-2-yl)-2,2,2-trifluoroethyl)amino)pyridin-2-yl)cyclopropane-1-carboxamide;
(1S,2S)—N-(4-(((8-acetamido-6-cyclopropylimidazo[1,2-a]pyridin-2-yl)methyl)amino)pyridin-2-yl)-2-(3-chlorophenyl)cyclopropane-1-carboxamide, formic acid salt (I-142);
(1S,2S)—N-(4-(((8-acetamido-6-cyclopropylimidazo[1,2-a]pyridin-2-yl)methyl)amino)pyridin-2-yl)-2-(3-chlorophenyl)cyclopropane-1-carboxamide;
rac-(1S*,2S*)-2-(5-chloro-2-cyanophenyl)-N-(4-(((6-cyclopropyl-8-(4-methylpiperazin-1-yl)imidazo[1,2-a]pyridin-2-yl)methyl)amino)pyridin-2-yl)cyclopropane-1-carboxamide (I-143);
(1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide (I-144);
(1S,2S)-2-(3-chlorophenyl)-N-(6-(((6-cyclopropyl-8-(3-fluoroazetidin-1-yl)imidazo[1,2-a]pyridin-2-yl)methyl)amino)pyrimidin-4-yl)cyclopropane-1-carboxamide (I-145);
methyl 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylate (I-146);
(1S,2S)-2-(3-chlorophenyl)-N-(6-(((6-cyclopropyl-8-((1-methyl-2-oxopyrrolidin-3-yl)oxy)imidazo[1,2-a]pyridin-2-yl)methyl)amino)pyrimidin-4-yl)cyclopropane-1-carboxamide (I-147);
(1S,2S)—N-(6-(((8-(benzyloxy)-6-cyclopropylimidazo[1,2-a]pyridin-2-yl)methyl)amino)pyrimidin-4-yl)-2-(3-chlorophenyl)cyclopropane-1-carboxamide (I-148);
(1S,2S)-2-(3-chlorophenyl)-N-(6-(((6-cyclopropyl-8-(3-oxopiperazin-1-yl)imidazo[1,2-a]pyridin-2-yl)methyl)amino)pyrimidin-4-yl)cyclopropane-1-carboxamide (I-149);
(1S,2S)-2-(3-chlorophenyl)-N-(6-(((6-cyclopropyl-8-(2-methoxypyridin-3-yl)imidazo[1,2-a]pyridin-2-yl)methyl)amino)pyrimidin-4-yl)cyclopropane-1-carboxamide (I-150);
(1S,2S)-2-(3-chlorophenyl)-N-(6-(((6-cyclopropyl-8-(N,N-dimethylsulfamoyl)imidazo[1,2-a]pyridin-2-yl)methyl)amino)pyrimidin-4-yl)cyclopropane-1-carboxamide (I-151);
(1S,2S)-2-(3-chlorophenyl)-N-(6-(((6-cyclopropyl-3-fluoroimidazo[1,2-a]pyridin-2-yl)methyl)amino)pyrimidin-4-yl)cyclopropane-1-carboxamide (I-152);
(1S,2S)-2-(3-chlorophenyl)-N-(6-(((6-cyclopropyl-8-(4-methyl-3-oxopiperazin-1-yl)imidazo[1,2-a]pyridin-2-yl)methyl)amino)pyrimidin-4-yl)cyclopropane-1-carboxamide (I-153);
(1S,2S)-2-(3-chlorophenyl)-N-(6-(((6-cyclopropyl-8-morpholinoimidazo[1,2-a]pyridin-2-yl)methyl)amino)pyrimidin-4-yl)cyclopropane-1-carboxamide (I-154);
(1S,2S)-2-(3-chlorophenyl)-N-(6-(((6-cyclopropyl-8-(3-oxothiomorpholino)imidazo[1,2-a]pyridin-2-yl)methyl)amino)pyrimidin-4-yl)cyclopropane-1-carboxamide (I-155);
tert-butyl (2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)carbamate (I-156);
(1S,2S)-2-(3-chlorophenyl)-N-(6-(((6-cyclopropyl-8-(3-methyl-2-oxoimidazolidin-1-yl)imidazo[1,2-a]pyridin-2-yl)methyl)amino)pyrimidin-4-yl)cyclopropane-1-carboxamide (I-157);
(1S,2S)-2-(3-chlorophenyl)-N-(6-(((6-cyclopropyl-8-(2-(trifluoromethyl)pyrrolidin-1-yl)imidazo[1,2-a]pyridin-2-yl)methyl)amino)pyrimidin-4-yl)cyclopropane-1-carboxamide (I-158);
(1S,2S)-2-(3-chlorophenyl)-N-(6-(((6-cyclopropyl-8-(2-hydroxypropan-2-yl)imidazo[1,2-a]pyridin-2-yl)methyl)amino)pyrimidin-4-yl)cyclopropane-1-carboxamide (I-159);
(1S,2S)-2-(3-chlorophenyl)-N-(6-(((6-isopropylimidazo[1,2-a]pyridin-2-yl)methyl)amino)pyrimidin-4-yl)cyclopropane-1-carboxamide (I-160);
(1S,2S)—N-(6-(((6-cyclopropyl-8-(methylsulfonyl)imidazo[1,2-a]pyridin-2-yl)methyl)(methyl)amino)-2-methylpyrimidin-4-yl)-2-(4-methylpyrimidin-2-yl)cyclopropane-1-carboxamide (I-161);
(1S,2S)-2-(3-chlorophenyl)-N-(6-(((6-cyclopropyl-8-(methylsulfonyl)imidazo[1,2-a]pyridin-2-yl)methyl)amino)pyrimidin-4-yl)cyclopropane-1-carboxamide (I-162);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(fluoromethyl)pyrimidin-2-yl)cyclopropane-1-carboxamide (I-163);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(difluoromethyl)pyrimidin-2-yl)cyclopropane-1-carboxamide (I-164);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(trifluoromethyl)pyrimidin-2-yl)cyclopropane-1-carboxamide (I-165);
rac-(1S*,2S*)-2-(3-cyano-6-methylpyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-166);
rac-(1S*,2S*)-2-(6-chloro-3-cyanopyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-167);
rac-(1S*,2S*)-2-(4-chloropyrimidin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-168);
(1S,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-169);
rac-(1 S*,2S*)-2-(5-chloro-2-cyanophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-170);
(1S,2S)-2-(3-chlorophenyl)-N-(6-(((6-cyclopropyl-8-(3-methyl-2,5-dioxoimidazolidin-1-yl)imidazo[1,2-a]pyridin-2-yl)methyl)amino)pyrimidin-4-yl)cyclopropane-1-carboxamide (I-171);
(1S,2S)-2-(4-chloropyridin-2-yl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide (I-172);
rac-(1S*,2S*)-2-(4-chloropyridin-2-yl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide (I-173);
(1S,2S)-2-(3-chlorophenyl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide (I-174);
(1S,2S)-2-(3-chlorophenyl)-N-(6-(((6-cyclopropylimidazo[1,2-a]pyridin-2-yl)methyl)amino)pyrimidin-4-yl)-N-hydroxycyclopropane-1-carboxamide (I-175);
rac-(1S*,2S*)-2-(6-chloropyrimidin-4-yl)-N-(6-(((6-cyclopropylimidazo[1,2-a]pyridin-2-yl)methyl)amino)pyrimidin-4-yl)cyclopropane-1-carboxamide (I-176);
rac-(1S*,2S*)-2-(4-chloropyrimidin-2-yl)-N-(6-(((6-cyclopropylimidazo[1,2-a]pyridin-2-yl)methyl)amino)pyrimidin-4-yl)cyclopropane-1-carboxamide (I-177);
rac-(1S*,2S*)-2-(5-chloropyridazin-3-yl)-N-(6-(((6-cyclopropylimidazo[1,2-a]pyridin-2-yl)methyl)amino)pyrimidin-4-yl)cyclopropane-1-carboxamide (I-178);
rac-(1S*,2S*)-2-(5-chloro-3-cyanothiophen-2-yl)-N-(6-(((6-cyclopropylimidazo[1,2-a]pyridin-2-yl)methyl)amino)pyrimidin-4-yl)cyclopropane-1-carboxamide (I-179);
rac-(1R*,2S*,3R*)-2-(3-chlorophenyl)-N-(6-(((6-cyclopropylimidazo[1,2-a]pyridin-2-yl)methyl)amino)pyrimidin-4-yl)-3-(hydroxymethyl)cyclopropane-1-carboxamide (I-180);
rac-(1S*,2S*)—N-(6-(((6-cyclopropylimidazo[1,2-a]pyridin-2-yl)methyl)amino)pyrimidin-4-yl)-2-(5-methoxy-2-oxopyridin-1(2H)-yl)cyclopropane-1-carboxamide (I-181);
rac-tert-butyl (4-((1S*,2S*)-2-((6-(((6-cyclopropylimidazo[1,2-a]pyridin-2-yl)methyl)amino)pyrimidin-4-yl)carbamoyl)cyclopropyl)-6-methoxypyridin-2-yl)carbamate (I-182);
rac-(1R*,2R*)-6′-chloro-N-(6-(((6-cyclopropylimidazo[1,2-a]pyridin-2-yl)methyl)amino)pyrimidin-4-yl)-2′,3′-dihydrospiro[cyclopropane-1,1′-indene]-2-carboxamide (I-183);
rac-(1S*,2S*)-2-(3-chlorophenyl)-N-(6-(((6-cyclopropylimidazo[1,2-a]pyridin-2-yl)methyl)amino)pyrimidin-4-yl)-1-fluorocyclopropane-1-carboxamide (I-184);
rac-(1S*,2S*)-2-(5-chlorothiophen-2-yl)-N-(6-(((6-cyclopropylimidazo[1,2-a]pyridin-2-yl)methyl)amino)pyrimidin-4-yl)cyclopropane-1-carboxamide (I-185);
rac-(1S*,2S*)-2-(4-chlorothiophen-2-yl)-N-(6-(((6-cyclopropylimidazo[1,2-a]pyridin-2-yl)methyl)amino)pyrimidin-4-yl)cyclopropane-1-carboxamide (I-186);
rac-(1S*,2S*)-2-(2-chloro-6-methoxypyridin-4-yl)-N-(6-(((6-cyclopropylimidazo[1,2-a]pyridin-2-yl)methyl)amino)pyrimidin-4-yl)cyclopropane-1-carboxamide (I-187);
rac-tert-butyl (2-chloro-4-((1S*,2S*)-2-((6-(((6-cyclopropylimidazo[1,2-a]pyridin-2-yl)methyl)amino)pyrimidin-4-yl)carbamoyl)cyclopropyl)phenyl)carbamate (I-188);
rac-tert-butyl (3-chloro-5-((1S*,2S*)-2-((6-(((6-cyclopropylimidazo[1,2-a]pyridin-2-yl)methyl)amino)pyrimidin-4-yl)carbamoyl)cyclopropyl)phenyl)carbamate (I-189);
rac-(1S*,2S*)-2-(3-chloro-2-nitrophenyl)-N-(6-(((6-cyclopropylimidazo[1,2-a]pyridin-2-yl)methyl)amino)pyrimidin-4-yl)cyclopropane-1-carboxamide (I-190);
rac-(1S*,2S*)-2-(5-chloro-1H-indazol-3-yl)-N-(6-(((6-cyclopropylimidazo[1,2-a]pyridin-2-yl)methyl)amino)pyrimidin-4-yl)cyclopropane-1-carboxamide (I-191);
rac-(1S*,2S*)-2-(4-chloropyridin-2-yl)-N-(6-(((6-cyclopropyl-8-(4-methylpiperazin-1-yl)imidazo[1,2-a]pyridin-2-yl)methyl)amino)pyrimidin-4-yl)cyclopropane-1-carboxamide (I-192);
(1S,2S)-2-(3-chlorophenyl)-N-(6-(((6-cyclopropyl-8-(1-methylazetidin-3-yl)imidazo[1,2-a]pyridin-2-yl)methyl)amino)pyrimidin-4-yl)cyclopropane-1-carboxamide (I-193);
(1S,2S)-2-(3-chlorophenyl)-N-(6-(((6-cyclopropyl-8-(3-methyl-2,4-dioxotetrahydropyrimidin-1(2H)-yl)imidazo[1,2-a]pyridin-2-yl)methyl)amino)pyrimidin-4-yl)cyclopropane-1-carboxamide (I-194);
rac-(1S*,2S*)-2-(4-chloropyridin-2-yl)-N-(6-(((6-cyclopropyl-8-(4-methyl-2-oxopiperazin-1-yl)imidazo[1,2-a]pyridin-2-yl)methyl)amino)pyrimidin-4-yl)cyclopropane-1-carboxamide (I-195);
(1S,2S)-2-(3-chlorophenyl)-N-(6-(((6-cyclopropyl-8-(7-hydroxyhexahydropyrrolo[1,2-a]pyrazin-2(1H)-yl)imidazo[1,2-a]pyridin-2-yl)methyl)amino)pyrimidin-4-yl)cyclopropane-1-carboxamide (I-196);
(1S,2S)-2-(3-chlorophenyl)-N-(6-(((6-cyclopropyl-8-(7,7-difluorohexahydropyrrolo[1,2-a]pyrazin-2(1H)-yl)imidazo[1,2-a]pyridin-2-yl)methyl)amino)pyrimidin-4-yl)cyclopropane-1-carboxamide (I-197);
(1S,2S)-2-(3-chlorophenyl)-N-(6-(((6-cyclopropyl-8-(3-hydroxy-1-methylazetidin-3-yl)imidazo[1,2-a]pyridin-2-yl)methyl)amino)pyrimidin-4-yl)cyclopropane-1-carboxamide (I-198);
(1S,2S)-2-(3-chlorophenyl)-N-(6-(((6-cyclopropyl-8-(5,5-dimethyl-2-oxooxazolidin-3-yl)imidazo[1,2-a]pyridin-2-yl)methyl)amino)pyrimidin-4-yl)cyclopropane-1-carboxamide (I-199);
(1S,2S)-2-(3-chlorophenyl)-N-(6-(((6-cyclopropyl-8-(hexahydropyrrolo[1,2-a]pyrazin-2(1H)-yl)imidazo[1,2-a]pyridin-2-yl)methyl)amino)pyrimidin-4-yl)cyclopropane-1-carboxamide (I-200);
(1S,2S)-2-(3-chlorophenyl)-N-(6-(((6-cyclopropyl-8-(4-cyclopropylpiperazin-1-yl)imidazo[1,2-a]pyridin-2-yl)methyl)amino)pyrimidin-4-yl)cyclopropane-1-carboxamide (I-201);
(1S,2S)-2-(3-chlorophenyl)-N-(6-(((6-cyclopropyl-8-(4-(oxetan-3-yl)piperazin-1-yl)imidazo[1,2-a]pyridin-2-yl)methyl)amino)pyrimidin-4-yl)cyclopropane-1-carboxamide (I-202);
(1S,2S)—N-(6-(((6-chloroimidazo[1,2-a]pyridin-2-yl)methyl)amino)pyrimidin-4-yl)-2-(3-chlorophenyl)cyclopropane-1-carboxamide (I-203);
rac-(1S*,2S*)—N-(6-(((6-cyclopropylimidazo[1,2-a]pyridin-2-yl)methyl)amino)pyrimidin-4-yl)-2-(4-methoxypyridin-2-yl)cyclopropane-1-carboxamide (I-204);
(1S,2S)-2-(3-chlorophenyl)-N-(6-(((6-(dimethylamino)imidazo[1,2-a]pyridin-2-yl)methyl)amino)pyrimidin-4-yl)cyclopropane-1-carboxamide (I-205);
rac-(1S*,2S*)-2-(2-cyano-5-methoxyphenyl)-N-(4-(((6-cyclopropylimidazo[1,2-a]pyridin-2-yl)methyl)amino)pyridin-2-yl)cyclopropane-1-carboxamide (I-206);
(1S,2S)—N-(6-(((8-acetyl-6-cyclopropylimidazo[1,2-a]pyridin-2-yl)methyl)amino)pyrimidin-4-yl)-2-(3-chlorophenyl)cyclopropane-1-carboxamide (I-207);
rac-(1S*,2S*)—N-(4-(((6-cyclopropyl-8-(3-methyl-2,4-dioxoimidazolidin-1-yl)imidazo[1,2-a]pyridin-2-yl)methyl)amino)pyridin-2-yl)-2-(4-methylpyrimidin-2-yl)cyclopropane-1-carboxamide (I-208);
(1S,2S)-2-(3-chlorophenyl)-N-(4-(((6-cyclopropyl-8-(3-methyl-2,4-dioxoimidazolidin-1-yl)imidazo[1,2-a]pyridin-2-yl)methyl)amino)pyridin-2-yl)cyclopropane-1-carboxamide (I-209);
rac-(1S*,2S*)-2-(6-chloropyrimidin-4-yl)-N-(4-(((6-cyclopropylimidazo[1,2-a]pyridin-2-yl)methyl)amino)pyridin-2-yl)cyclopropane-1-carboxamide (I-210);
rac-(1S*,2S*)-2-(5-chloropyridazin-3-yl)-N-(4-(((6-cyclopropylimidazo[1,2-a]pyridin-2-yl)methyl)amino)pyridin-2-yl)cyclopropane-1-carboxamide (I-211);
rac-(1S*,2S*)-2-(5-chloro-3-cyanothiophen-2-yl)-N-(4-(((6-cyclopropylimidazo[1,2-a]pyridin-2-yl)methyl)amino)pyridin-2-yl)cyclopropane-1-carboxamide (I-212);
rac-(1R*,2S*,3R*)-2-(3-chlorophenyl)-N-(4-(((6-cyclopropylimidazo[1,2-a]pyridin-2-yl)methyl)amino)pyridin-2-yl)-3-(hydroxymethyl)cyclopropane-1-carboxamide (I-213);
rac-(1S*,2S*)—N-(4-(((6-cyclopropylimidazo[1,2-a]pyridin-2-yl)methyl)amino)pyridin-2-yl)-2-(4-methoxypyridin-2-yl)cyclopropane-1-carboxamide (I-214);
rac-tert-butyl (4-((1S*,2S*)-2-((4-(((6-cyclopropylimidazo[1,2-a]pyridin-2-yl)methyl)amino)pyridin-2-yl)carbamoyl)cyclopropyl)-6-methoxypyridin-2-yl)carbamate (I-216);
rac-(1S*,2S*)-2-(3-chlorophenyl)-N-(4-(((6-cyclopropylimidazo[1,2-a]pyridin-2-yl)methyl)amino)pyridin-2-yl)-1-fluorocyclopropane-1-carboxamide (I-217);
rac-(1S*,2S*)-2-(5-chlorothiophen-2-yl)-N-(4-(((6-cyclopropylimidazo[1,2-a]pyridin-2-yl)methyl)amino)pyridin-2-yl)cyclopropane-1-carboxamide (I-218);
rac-(1S*,2S*)-2-(4-chlorothiophen-2-yl)-N-(4-(((6-cyclopropylimidazo[1,2-a]pyridin-2-yl)methyl)amino)pyridin-2-yl)cyclopropane-1-carboxamide (I-219);
rac-(1S*,2S*)-2-(2-chloropyridin-4-yl)-N-(4-(((6-cyclopropylimidazo[1,2-a]pyridin-2-yl)methyl)amino)pyridin-2-yl)cyclopropane-1-carboxamide (I-220);
rac-(1S*,2S*)-2-(5-chloropyridin-3-yl)-N-(4-(((6-cyclopropylimidazo[1,2-a]pyridin-2-yl)methyl)amino)pyridin-2-yl)cyclopropane-1-carboxamide (I-221);
rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide (I-222);
rac-(1S*,2S*)-2-(5-chloro-2-nitrophenyl)-N-(4-(((6-cyclopropylimidazo[1,2-a]pyridin-2-yl)methyl)amino)pyridin-2-yl)cyclopropane-1-carboxamide (I-223);
rac-(1S*,2S*)-2-(2-cyano-5-methoxyphenyl)-N-(4-(((6-cyclopropylimidazo[1,2-a]pyridin-2-yl)methyl)amino)pyridin-2-yl)cyclopropane-1-carboxamide (I-224);
rac-(1S*,2S*)-2-(5-chloro-1H-indazol-3-yl)-N-(4-(((6-cyclopropylimidazo[1,2-a]pyridin-2-yl)methyl)amino)pyridin-2-yl)cyclopropane-1-carboxamide (I-225);
tert-butyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)azetidine-1-carboxylate (I-226);
(1S,2S)-2-(3-chlorophenyl)-N-(4-(((6-cyclopropyl-8-(3-fluoro-1-methylazetidin-3-yl)imidazo[1,2-a]pyridin-2-yl)methyl)amino)pyridin-2-yl)cyclopropane-1-carboxamide (I-227);
(1S,2S)-2-(3-chlorophenyl)-N-(4-(((6-cyclopropyl-8-(7-hydroxyhexahydropyrrolo[1,2-a]pyrazin-2(1H)-yl)imidazo[1,2-a]pyridin-2-yl)methyl)amino)pyridin-2-yl)cyclopropane-1-carboxamide (I-228);
(1S,2S)-2-(3-chlorophenyl)-N-(4-(((6-cyclopropyl-8-(4-fluoro-1-methylpiperidin-4-yl)imidazo[1,2-a]pyridin-2-yl)methyl)amino)pyridin-2-yl)cyclopropane-1-carboxamide (I-229);
(1S,2S)-2-(3-chlorophenyl)-N-(4-(((6-cyclopropyl-8-(7,7-difluorohexahydropyrrolo[1,2-a]pyrazin-2(1H)-yl)imidazo[1,2-a]pyridin-2-yl)methyl)amino)pyridin-2-yl)cyclopropane-1-carboxamide (I-230);
(1S,2S)-2-(3-chlorophenyl)-N-(4-(((6-cyclopropyl-8-(hexahydropyrrolo[1,2-a]pyrazin-2(1H)-yl)imidazo[1,2-a]pyridin-2-yl)methyl)amino)pyridin-2-yl)cyclopropane-1-carboxamide (I-231);
(1S,2S)-2-(3-chlorophenyl)-N-(4-(((6-cyclopropyl-8-(4-cyclopropylpiperazin-1-yl)imidazo[1,2-a]pyridin-2-yl)methyl)amino)pyridin-2-yl)cyclopropane-1-carboxamide (I-232);
(1S,2S)-2-(3-chlorophenyl)-N-(4-(((6-cyclopropyl-8-(4-(oxetan-3-yl)piperazin-1-yl)imidazo[1,2-a]pyridin-2-yl)methyl)amino)pyridin-2-yl)cyclopropane-1-carboxamide (I-233);
(1S,2S)-2-(3-chlorophenyl)-N-(4-(((6-cyclopropyl-8-(4-ethylpiperazin-1-yl)imidazo[1,2-a]pyridin-2-yl)methyl)amino)pyridin-2-yl)cyclopropane-1-carboxamide (I-234);
(1S,2S)-2-(3-chlorophenyl)-N-(4-(((6-cyclopropyl-8-(methylsulfonyl)imidazo[1,2-a]pyridin-2-yl)methyl)amino)pyridin-2-yl)cyclopropane-1-carboxamide (I-235);
(1S,2S)-2-(3-chlorophenyl)-N-(4-(((6-cyclopropyl-8-(1-hydroxyethyl)imidazo[1,2-a]pyridin-2-yl)methyl)amino)pyridin-2-yl)cyclopropane-1-carboxamide (I-236);
(1S,2S)—N-(4-(((8-acetyl-6-cyclopropylimidazo[1,2-a]pyridin-2-yl)methyl)amino)pyridin-2-yl)-2-(3-chlorophenyl)cyclopropane-1-carboxamide (I-237);
(1S,2S)-2-(3-chlorophenyl)-N-(4-(((6-cyclopropyl-8-(2-oxooxazolidin-3-yl)imidazo[1,2-a]pyridin-2-yl)methyl)amino)pyridin-2-yl)cyclopropane-1-carboxamide (I-238);
methyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate (I-239);
ethyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate (I-240);
ethyl 2-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)acetate (I-241);
rac-(1S*,2S*)-2-(3-chlorophenyl)-N-(4-(((6-cyclopropyl-8-(3-hydroxyoxetan-3-yl)imidazo[1,2-a]pyridin-2-yl)methyl)amino)pyridin-2-yl)cyclopropane-1-carboxamide (I-242);
(1S,2S)-2-(3-chlorophenyl)-N-(2-(((6-cyclopropyl-8-(4-methylpiperazin-1-yl)imidazo[1,2-a]pyridin-2-yl)methyl)amino)pyridin-4-yl)cyclopropane-1-carboxamide (I-243);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-methoxypyrimidin-2-yl)-2-(4-methylpyrimidin-2-yl)cyclopropane-1-carboxamide (I-244);
(1S,2S)—N-(6-(((R)-1-(6-cyclopropyl-8-(2,5-dimethyl-6-oxo-2,5,7-triazaspiro[3.4]octan-7-yl)imidazo[1,2-a]pyridin-2-yl)ethyl)amino)pyrimidin-4-yl)-2-(4-methylpyrimidin-2-yl)cyclopropane-1-carboxamide (I-245);
(1S,2S)—N-(2-(((R)-1-(6-cyclopropyl-8-(3-methyl-2,4-dioxoimidazolidin-1-yl)imidazo[1,2-a]pyridin-2-yl)ethyl)amino)-6-methylpyridin-4-yl)-2-(4-methylpyrimidin-2-yl)cyclopropane-1-carboxamide (I-246);
(1S,2S)—N-(6-chloro-5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclopropane-1-carboxamide (I-247);
(1S*,2S*)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide, isomer 1 (I-248);
(1S,2S)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1R,2R)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1S*,2S*)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide, isomer 2 (I-249);
(1S,2S)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1R,2R)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1S,2S)-2-(3-chlorophenyl)-N-(6-((2R,4R)-2-(6-cyclopropylimidazo[1,2-a]pyridin-2-yl)-4-hydroxypyrrolidin-1-yl)pyrimidin-4-yl)cyclopropane-1-carboxamide (I-250);
(1S,2S)-2-(3-chlorophenyl)-N-(6-((R)-2-(6-cyclopropylimidazo[1,2-a]pyridin-2-yl)pyrrolidin-1-yl)pyrimidin-4-yl)cyclopropane-1-carboxamide (I-251);
(1S,2S)—N-(4-(((6-chloro-8-(4-methylpiperazin-1-yl)imidazo[1,2-a]pyridin-2-yl)methyl)amino)pyridin-2-yl)-2-(3-chlorophenyl)cyclopropane-1-carboxamide (I-252);
(1S,2S)—N-(4-(((6-chloroimidazo[1,2-a]pyridin-2-yl)methyl)amino)pyridin-2-yl)-2-(3-chlorophenyl)cyclopropane-1-carboxamide (I-253);
(1S,2S)—N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)-2-(4-methylpyrimidin-2-yl)cyclopropane-1-carboxamide (I-254);
(1S,2S)-2-(3-chlorophenyl)-N-(6-(((6-cyclopropyl-8-(4-methylpiperazin-1-yl)imidazo[1,2-a]pyridin-2-yl)methyl)amino)pyrazin-2-yl)cyclopropane-1-carboxamide (I-255);
rac-(1S*,2S*)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide (I-256);
rac-(1S*,2S*)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide (I-257), first eluting isomer;
rac-(1S*,2S*)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide, second eluting isomer (I-258);
(1S,2S)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide;
(1R,2R)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-259);
(1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-260);
(1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-261);
rac-(1S*,2S*)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-262);
(1R,2R)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-263);
(1S,2S)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-264);
rac-(1S*,2S*)-2-(4-chloropyridin-2-yl)-N-(6-(((6-cyclopropylimidazo[1,2-a]pyridin-2-yl)methyl)amino)pyrimidin-4-yl)cyclopropane-1-carboxamide (I-265);
rac-(1S*,2S*)-2-(4-chloropyridin-2-yl)-N-(6-(((6-cyclopropylimidazo[1,2-a]pyridin-2-yl)methyl)amino)pyrimidin-4-yl)cyclopropane-1-carboxamide, first eluting isomer (I-266);
rac-(1S*,2S*)-2-(4-chloropyridin-2-yl)-N-(6-(((6-cyclopropylimidazo[1,2-a]pyridin-2-yl)methyl)amino)pyrimidin-4-yl)cyclopropane-1-carboxamide, second eluting isomer (I-267);
(1R,2R)-2-(4-chloropyridin-2-yl)-N-(6-(((6-cyclopropylimidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(4-chloropyridin-2-yl)-N-(4-(((6-cyclopropylimidazo[1,2-a]pyridin-2-yl)methyl)amino)pyridin-2-yl)cyclopropane-1-carboxamide (Example I-268);
rac-(1S*,2S*)-2-(4-chloropyridin-2-yl)-N-(4-(((6-cyclopropylimidazo[1,2-a]pyridin-2-yl)methyl)amino)pyridin-2-yl)cyclopropane-1-carboxamide, first eluting isomer (Example I-269);
rac-(1S*,2S*)-2-(4-chloropyridin-2-yl)-N-(4-(((6-cyclopropylimidazo[1,2-a]pyridin-2-yl)methyl)amino)pyridin-2-yl)cyclopropane-1-carboxamide, second eluting isomer (Example I-270);
(1R,2R)-2-(4-chloropyridin-2-yl)-N-(4-(((6-cyclopropylimidazo[1,2-a]pyridin-2-yl)methyl)amino)pyridin-2-yl)cyclopropane-1-carboxamide;
(1S,2S)-2-(4-chloropyridin-2-yl)-N-(4-(((6-cyclopropylimidazo[1,2-a]pyridin-2-yl)methyl)amino)pyridin-2-yl)cyclopropane-1-carboxamide;
rac-(1S*,2S*)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide (I-271);
rac-(1S*,2S*)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide, first eluting isomer (I-272);
rac-(1S*,2S*)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide, second eluting isomer (I-273);
(1R,2R)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide;
(1S,2S)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide;
(1S,2S)-2-(3-chlorophenyl)-N-(6-(((6-cyclopropyl-8-(N-methylacetamido)imidazo[1,2-a]pyridin-2-yl)methyl)amino)pyrimidin-4-yl)cyclopropane-1-carboxamide (I-274);
(1S,2S)—N-(6-(((8-acetamido-6-cyclopropylimidazo[1,2-a]pyridin-2-yl)methyl)amino)pyrimidin-4-yl)-2-(3-chlorophenyl)cyclopropane-1-carboxamide (I-275);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-(dimethylamino)ethoxy)pyrimidin-4-yl)-2-(4-methylpyrimidin-2-yl)cyclopropane-1-carboxamide (I-276);
rac-(1S*,2S*)-2-(2-amino-6-methoxypyridin-4-yl)-N-(6-(((6-cyclopropylimidazo[1,2-a]pyridin-2-yl)methyl)amino)pyrimidin-4-yl)cyclopropane-1-carboxamide (I-277);
rac-(1S*,2S*)-2-(4-amino-3-chlorophenyl)-N-(6-(((6-cyclopropylimidazo[1,2-a]pyridin-2-yl)methyl)amino)pyrimidin-4-yl)cyclopropane-1-carboxamide (I-278);
rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide (I-279);
rac-(1S*,2S*)-2-(2-amino-3-chlorophenyl)-N-(6-(((6-cyclopropylimidazo[1,2-a]pyridin-2-yl)methyl)amino)pyrimidin-4-yl)cyclopropane-1-carboxamide (I-280):
(1S,2S)-2-(3-chlorophenyl)-N-(6-(((6-cyclopropyl-8-(1-hydroxyethyl)imidazo[1,2-a]pyridin-2-yl)methyl)amino)pyrimidin-4-yl)cyclopropane-1-carboxamide (I-281);
(1S,2S)-2-(3-chlorophenyl)-N-(4-((2R,4S)-2-(6-cyclopropylimidazo[1,2-a]pyridin-2-yl)-4-hydroxypyrrolidin-1-yl)pyridin-2-yl)cyclopropane-1-carboxamide (I-282);
(1S,2S)-2-(3-chlorophenyl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide (I-283);
(1S,2S)—N-(4-(((8-(azetidin-3-yl)-6-cyclopropylimidazo[1,2-a]pyridin-2-yl)methyl)amino)pyridin-2-yl)-2-(3-chlorophenyl)cyclopropane-1-carboxamide (I-284);
(1S,2S)-2-(3-chlorophenyl)-N-(6-(((6-cyclopropyl-8-(4-methylpiperazin-1-yl)imidazo[1,2-a]pyridin-2-yl)methyl)amino)-2-hydroxypyrimidin-4-yl)cyclopropane-1-carboxamide (I-285);
rac-(1S*,2S*)-2-(2-amino-6-methoxypyridin-4-yl)-N-(4-(((6-cyclopropylimidazo[1,2-a]pyridin-2-yl)methyl)amino)pyridin-2-yl)cyclopropane-1-carboxamide (I-286);
rac-(1S*,2S*)-2-(5-chloro-2-(H-tetrazol-1-yl)phenyl)-N-(4-(((6-cyclopropylimidazo[1,2-a]pyridin-2-yl)methyl)amino)pyridin-2-yl)cyclopropane-1-carboxamide (I-287);
(1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (I-288);
rac-(1S*,2S*)-2-(5-chloro-2-cyanophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (I-289);
3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoic acid (I-290);
(1S,2S)-2-(3-chlorophenyl)-N-(4-(((6-cyclopropyl-8-(3-hydroxypropyl)imidazo[1,2-a]pyridin-2-yl)methyl)amino)pyridin-2-yl)cyclopropane-1-carboxamide (I-291);
(1S,2S)-2-(3-chlorophenyl)-N-(4-(((6-cyclopropyl-8-(2-hydroxyethyl)imidazo[1,2-a]pyridin-2-yl)methyl)amino)pyridin-2-yl)cyclopropane-1-carboxamide (I-292);
(1S,2S)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (I-293);
rac-(1S*,2S*)-2-(3-chlorophenyl)-N-(4-(((6-cyclopropyl-8-(3-fluorooxetan-3-yl)imidazo[1,2-a]pyridin-2-yl)methyl)amino)pyridin-2-yl)cyclopropane-1-carboxamide (I-294);
(1S,2S)-2-(3-chlorophenyl)-N-(4-(((6-cyclopropylimidazo[1,2-a]pyridin-2-yl)methyl)amino)-6-hydroxy-1,3,5-triazin-2-yl)cyclopropane-1-carboxamide (I-295);
(1S,2S)-2-(3-chlorophenyl)-N-(6-(((6-cyclopropylimidazo[1,2-a]pyridin-2-yl)methyl)amino)-2-oxo-2,3-dihydropyrimidin-4-yl)cyclopropane-1-carboxamide (I-296);
(1S,2S)—N-(6-((2R,4S)-4-amino-2-(6-cyclopropylimidazo[1,2-a]pyridin-2-yl)pyrrolidin-1-yl)pyrimidin-4-yl)-2-(3-chlorophenyl)cyclopropane-1-carboxamide (I-297);
2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid (I-298);
2-(((6-((1S,2S)-2-(3-chlorophenyl)cyclopropane-1-carboxamido)pyrimidin-4-yl)amino)methyl)-6-cyclopropyl-N,N-dimethylimidazo[1,2-a]pyridine-8-carboxamide (I-299);
(1S,2S)-2-(3-chlorophenyl)-N-(6-(((6-cyclopropyl-8-(morpholine-4-carbonyl)imidazo[1,2-a]pyridin-2-yl)methyl)amino)pyrimidin-4-yl)cyclopropane-1-carboxamide (I-300);
(1S,2S)-2-(3-chlorophenyl)-N-(6-(((6-cyclopropyl-8-(4-methylpiperazine-1-carbonyl)imidazo[1,2-a]pyridin-2-yl)methyl)amino)pyrimidin-4-yl)cyclopropane-1-carboxamide (I-301);
2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxamide (I-302);
2-(((6-((1S,2S)-2-(3-chlorophenyl)cyclopropane-1-carboxamido)pyrimidin-4-yl)amino)methyl)-6-cyclopropyl-N-methylimidazo[1,2-a]pyridine-8-carboxamide (I-303);
(1S,2S)-2-(3-chlorophenyl)-N-(6-(((6-cyclopropyl-8-(piperazine-1-carbonyl)imidazo[1,2-a]pyridin-2-yl)methyl)amino)pyrimidin-4-yl)cyclopropane-1-carboxamide (I-304);
(1S,2S)-2-(3-chlorophenyl)-N-(6-(((6-cyclopropyl-8-(2-oxo-1,2-dihydropyridin-3-yl)imidazo[1,2-a]pyridin-2-yl)methyl)amino)pyrimidin-4-yl)cyclopropane-1-carboxamide (I-305);
(1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide (I-306);
(1S,2S)-2-(3-chlorophenyl)-N-(6-(((6-cyclopropyl-8-(piperazin-1-yl)imidazo[1,2-a]pyridin-2-yl)methyl)amino)pyrimidin-4-yl)cyclopropane-1-carboxamide (I-307);
(1S,2S)—N-(6-(((8-amino-6-cyclopropylimidazo[1,2-a]pyridin-2-yl)methyl)amino)pyrimidin-4-yl)-2-(3-chlorophenyl)cyclopropane-1-carboxamide (I-308);
(1S,2S)-2-(3-chlorophenyl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide (I-309);
rac-(1S*,2S*)-2-(5-chloro-2-(2-methoxyethoxy)phenyl)-N-(6-(((6-cyclopropylimidazo[1,2-a]pyridin-2-yl)methyl)amino)pyrimidin-4-yl)cyclopropane-1-carboxamide, formic acid salt (I-310);
rac-(1S*,2S*)-2-(5-chloro-2-(2-methoxyethoxy)phenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-(difluoromethoxy)phenyl)-N-(6-(((6-cyclopropylimidazo[1,2-a]pyridin-2-yl)methyl)amino)pyrimidin-4-yl)cyclopropane-1-carboxamide (I-311);
rac-ethyl 3-(4-chloro-2-((1S*,2S*)-2-((6-(((6-cyclopropylimidazo[1,2-a]pyridin-2-yl)methyl)amino)pyrimidin-4-yl)carbamoyl)cyclopropyl)phenoxy)propanoate (I-312);
rac-(1S*,2S*)-2-(5-chloro-2-hydroxyphenyl)-N-(6-(((6-cyclopropylimidazo[1,2-a]pyridin-2-yl)methyl)amino)pyrimidin-4-yl)cyclopropane-1-carboxamide (I-313);
rac-(1S*,2S*)-2-(6-cyano-2-fluoro-3-methoxyphenyl)-N-(6-(((6-cyclopropylimidazo[1,2-a]pyridin-2-yl)methyl)amino)pyrimidin-4-yl)cyclopropane-1-carboxamide, formic acid salt (I-314);
rac-(1S*,2S*)-2-(6-cyano-2-fluoro-3-methoxyphenyl)-N-(6-(((6-cyclopropylimidazo[1,2-a]pyridin-2-yl)methyl)amino)pyrimidin-4-yl)cyclopropane-1-carboxamide;
rac-3-(4-chloro-2-((1S*,2S*)-2-((6-(((6-cyclopropylimidazo[1,2-a]pyridin-2-yl)methyl)amino)pyrimidin-4-yl)carbamoyl)cyclopropyl)phenoxy)acetic acid (I-315);
rac-(1S*,2S*)-2-(5-chloro-2-(H-tetrazol-1-yl)phenyl)-N-(6-(((6-cyclopropylimidazo[1,2-a]pyridin-2-yl)methyl)amino)pyrimidin-4-yl)cyclopropane-1-carboxamide (I-316);
(rac-(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide (I-317);
(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, first eluting isomer (I-318);
(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, second eluting isomer (I-319);
(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, third eluting isomer (I-320);
(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, fourth eluting isomer (I-321);
(1S,2S)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-322);
(1R,2R)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-323);
(1S,2S)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-324);
(1S,2S)-2-(4-chloropyridin-2-yl)-N-(6-(((6-cyclopropyl-8-(methylsulfonyl)imidazo[1,2-a]pyridin-2-yl)methyl)amino)pyrimidin-4-yl)cyclopropane-1-carboxamide (I-325);
(1S,2S)-2-(4-chloropyridin-2-yl)-N-(6-(((6-cyclopropyl-8-(1,1-dioxidothiomorpholino)imidazo[1,2-a]pyridin-2-yl)methyl)amino)pyrimidin-4-yl)cyclopropane-1-carboxamide (I-326);
(1S,2S)-2-(4-chloropyridin-2-yl)-N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)cyclopropane-1-carboxamide (I-327);
(1S,2S)-2-(4-chloropyridin-2-yl)-N-(6-(((6-cyclopropylimidazo[1,2-a]pyridin-2-yl)methyl)(2-hydroxyethyl)amino)pyrimidin-4-yl)cyclopropane-1-carboxamide (I-328);
rac-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide (I-329);
rac-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, first eluting mixture of enantiomers (I-330);
rac-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, second eluting mixture of enantiomers (I-331);
2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, first eluting enantiomer (I-332);
2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, second eluting enantiomer (I-333);
(1R,2R)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide;
(1S,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide;
(1R,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide;
(1S,2R)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide;
(rac-(1S*,2S*))-1-(2-(((6-chloropyrimidin-4-yl)amino)methyl)-6-cyclopropylimidazo[1,2-a]pyridin-8-yl)pyrrolidin-2-one (I-334);
(rac-(1S*,2S*))-1-(2-(((6-chloropyrimidin-4-yl)amino)methyl)-6-cyclopropylimidazo[1,2-a]pyridin-8-yl)pyrrolidin-2-one, first eluting isomer (I-335);
(rac-(1S*,2S*))-1-(2-(((6-chloropyrimidin-4-yl)amino)methyl)-6-cyclopropylimidazo[1,2-a]pyridin-8-yl)pyrrolidin-2-one, second eluting isomer (I-336);
(1S,2S)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
(1R,2R)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
(rac-(1S*,2S*))-2-(5-chloro-2-cyanophenyl)-N-(6-(((6-cyclopropyl-8-(2-hydroxypropan-2-yl)imidazo[1,2-a]pyridin-2-yl)methyl)amino)pyrimidin-4-yl)cyclopropane-1-carboxamide (I-337);
(1S,2S)-2-(3-chlorophenyl)-N-(4-(((6-cyclopropylimidazo[1,2-a]pyridin-2-yl)methyl)amino)-6-methylpyridin-2-yl)cyclopropane-1-carboxamide (I-338);
(1S,2S)-2-(4-chloropyridin-2-yl)-N-(4-(((6-cyclopropylimidazo[1,2-a]pyridin-2-yl)methyl)amino)-6-methoxypyridin-2-yl)cyclopropane-1-carboxamide (I-339);
(1S,2S)-2-(3-chlorophenyl)-N-(4-(((6-cyclopropylimidazo[1,2-a]pyridin-2-yl)methyl)amino)-6-methoxypyridin-2-yl)cyclopropane-1-carboxamide (I-340);
(1S,2S)-2-(3-chlorophenyl)-N-(4-(((6-cyclopropylimidazo[1,2-a]pyridin-2-yl)methyl)amino)-6-(2-morpholinoethyl)pyridin-2-yl)cyclopropane-1-carboxamide (I-341);
rac-(1S*,2S*)-2-(5-chloro-2-cyanophenyl)-N-(4-(((6-cyclopropylimidazo[1,2-a]pyridin-2-yl)methyl)amino)-6-methylpyridin-2-yl)cyclopropane-1-carboxamide (I-342);
(1S,2S)-2-(4-chloropyridin-2-yl)-N-(4-(((6-cyclopropylimidazo[1,2-a]pyridin-2-yl)methyl)amino)-6-oxo-1,6-dihydropyridin-2-yl)cyclopropane-1-carboxamide (I-343);
(rac-(1S*,2S*))-2-(3-chlorophenyl)-N-(4-(((6-cyclopropylimidazo[1,2-a]pyridin-2-yl)methyl)amino)-1,3,5-triazin-2-yl)cyclopropane-1-carboxamide (I-344);
((rac-(1S*,2S*))-2-(3-chlorophenyl)-N-(6-(((6-cyclopropylimidazo[1,2-a]pyridin-2-yl)methyl)amino)pyrazin-2-yl)cyclopropane-1-carboxamide (I-345);
(rac-(1 S*,2S*))-2-(3-chlorophenyl)-N-(4-(((6-cyclopropylimidazo[1,2-a]pyridin-2-yl)methyl)amino)pyrimidin-2-yl)cyclopropane-1-carboxamide (I-346);
(rac-(1S*,2S*))-2-(3-chlorophenyl)-N-(2-(((6-cyclopropylimidazo[1,2-a]pyridin-2-yl)methyl)amino)pyrimidin-4-yl)cyclopropane-1-carboxamide (I-347);
(rac-(1S*,2S*))-2-(3-chlorophenyl)-N-(6-(((6-cyclopropylimidazo[1,2-a]pyridin-2-yl)methyl)amino)pyridazin-4-yl)cyclopropane-1-carboxamide (I-348);
2-(3-chlorophenyl)-2-cyano-N-(6-(((6-cyclopropylimidazo[1,2-a]pyridin-2-yl)methyl)amino)pyrimidin-4-yl)cyclopropane-1-carboxamide (I-349);
(1S,2S)-2-(3-chlorophenyl)-N-(6-(((6-cyclopropylimidazo[1,2-a]pyridin-2-yl)methyl)amino)pyrimidin-4-yl)-N-methylcyclopropane-1-carboxamide (I-350);
rac-(1S*,2S*)—N-(6-(((6-cyclopropylimidazo[1,2-a]pyridin-2-yl)methyl)amino)pyrimidin-4-yl)-2-(3-cyclopropylphenyl)cyclopropane-1-carboxamide (I-351);
rac-(1S*,2S*)-2-(5-chloro-2-cyanophenyl)-N-(6-((6-cyclopropylimidazo[1,2-a]pyridin-2-yl)(hydroxy)methyl)pyrimidin-4-yl)cyclopropane-1-carboxamide (I-352);
rac-(1 S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide (I-353);
(1S,2S)—N-(6-(((6-cyclopropyl-8-(3-methyl-2,4-dioxoimidazolidin-1-yl)imidazo[1,2-a]pyridin-2-yl)methyl)(methyl)amino)-2-(methoxymethyl)pyrimidin-4-yl)-2-(4-methylpyrimidin-2-yl)cyclopropane-1-carboxamide (I-354);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(methoxymethyl)pyrimidin-4-yl)-2-(4-methylpyrimidin-2-yl)cyclopropane-1-carboxamide (I-355);
(1S,2S)—N-(6-((1-(6-chloro-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-356);
(1S,2S)—N-(6-(((6-cyclopropyl-8-(6-methyl-5,7-dioxo-4,6-diazaspiro[2.4]heptan-4-yl)imidazo[1,2-a]pyridin-2-yl)methyl)amino)-2-methylpyrimidin-4-yl)-2-(4-methylpyrimidin-2-yl)cyclopropane-1-carboxamide (I-357);
(1S,2S)—N-(6-(((R)-1-(6-cyclopropyl-8-(3-methyl-2,4-dioxoimidazolidin-1-yl)imidazo[1,2-a]pyridin-2-yl)propyl)amino)-2-methylpyrimidin-4-yl)-2-(4-methylpyrimidin-2-yl)cyclopropane-1-carboxamide (I-358);
(1S,2S)—N-(6-(((6-cyclopropyl-8-(3-(2-hydroxy-2-methylpropyl)-2,4-dioxoimidazolidin-1-yl)imidazo[1,2-a]pyridin-2-yl)methyl)(methyl)amino)-2-methylpyrimidin-4-yl)-2-(4-methylpyrimidin-2-yl)cyclopropane-1-carboxamide (I-359);
(1S,2S)—N-(6-(((6-cyclopropyl-8-(3-methyl-2,4-dioxoimidazolidin-1-yl)imidazo[1,2-a]pyridin-2-yl)methyl)(ethyl)amino)-2-methylpyrimidin-4-yl)-2-(4-methylpyrimidin-2-yl)cyclopropane-1-carboxamide (I-360);
(1S,2S)—N-(6-(((R)-1-(6-cyclopropyl-8-(methylsulfonyl)imidazo[1,2-a]pyridin-2-yl)ethyl)amino)-2-methylpyrimidin-4-yl)-2-(4-methylpyrimidin-2-yl)cyclopropane-1-carboxamide (I-361);
(1S,2S)—N-(6-((1-(7-cyclopropyl-5-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-362);
(1S,2S)—N-(6-((1-(6-cyclopropyl-8-(methylsulfonyl)imidazo[1,2-a]pyridin-2-yl)ethyl)amino)-2-methylpyrimidin-4-yl)-2-(4-methylpyrimidin-2-yl)cyclopropane-1-carboxamide (I-363);
(1S,2S)—N-(6-((1-(6-cyclopropyl-8-(3-methyl-2-oxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-364);
(1S,2S)—N-(6-(((6-cyclopropyl-8-(3-methyl-2,4-dioxoimidazolidin-1-yl)imidazo[1,2-a]pyridin-2-yl)methyl)amino)-2-methylpyrimidin-4-yl)-2-(4-methylpyrimidin-2-yl)cyclopropane-1-carboxamide (I-365);
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-methylpyrimidin-4-yl)-2-(4-methylpyrimidin-2-yl)cyclopropane-1-carboxamide (I-366);
(1S,2S)—N-(6-(((6-cyclopropyl-8-(2,4-dimethyl-3,5-dioxo-1,2,4-triazolidin-1-yl)imidazo[1,2-a]pyridin-2-yl)methyl)amino)pyrimidin-4-yl)-2-(4-methylpyrimidin-2-yl)cyclopropane-1-carboxamide (I-367);
(1S,2S)—N-(6-(((6-cyclopropyl-8-(3-(2-hydroxy-2-methylpropyl)-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide (I-368);
(1S,2S)—N-(6-((1-(6-cyclopropyl-8-(3-methyl-2,4-dioxoimidazolidin-1-yl)imidazo[1,2-a]pyridin-2-yl)ethyl)(methyl)amino)pyrimidin-4-yl)-2-(4-methylpyrimidin-2-yl)cyclopropane-1-carboxamide (I-369);
(1S,2S)—N-(6-((1-(6-cyclopropyl-8-(3-methyl-2,4-dioxoimidazolidin-1-yl)imidazo[1,2-a]pyridin-2-yl)-2,2,2-trifluoroethyl)amino)pyrimidin-4-yl)-2-(4-methylpyrimidin-2-yl)cyclopropane-1-carboxamide (I-370);
benzyl N-((6-cyclopropyl-8-(3-methyl-2,4-dioxoimidazolidin-1-yl)imidazo[1,2-a]pyridin-2-yl)methyl)-N-(6-((1S,2S)-2-(4-methylpyrimidin-2-yl)cyclopropane-1-carboxamido)pyrimidin-4-yl)glycinate (I-371);
(1S,2S)—N-(6-(((6-cyclopropyl-8-(6-oxo-2,5-dioxa-7-azaspiro[3.4]octan-7-yl)imidazo[1,2-a]pyridin-2-yl)methyl)amino)pyrimidin-4-yl)-2-(4-methylpyrimidin-2-yl)cyclopropane-1-carboxamide (I-372);
(1S,2S)—N-(6-((1-(6-cyclopropyl-8-(3-methyl-2,4-dioxoimidazolidin-1-yl)imidazo[1,2-a]pyridin-2-yl)-2-methoxyethyl)amino)pyrimidin-4-yl)-2-(4-methylpyrimidin-2-yl)cyclopropane-1-carboxamide (I-373);
(1S,2S)—N-(6-(((6-(1-fluorocyclopropyl)-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-374);
(1S,2S)—N-(6-(((6-cyclopropyl-8-(2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide (I-375);
(1S,2S)—N-(6-(((6-cyclopropyl-8-(2-oxooxazolidin-3-yl)imidazo[1,2-a]pyridin-2-yl)methyl)amino)pyrimidin-4-yl)-2-(4-methylpyrimidin-2-yl)cyclopropane-1-carboxamide (I-376);
(1S,2S)—N-(6-(((6-cyclopropyl-8-(2-oxopyrrolidin-1-yl)imidazo[1,2-a]pyridin-2-yl)methyl)amino)pyrimidin-4-yl)-2-(4-methylpyrimidin-2-yl)cyclopropane-1-carboxamide (I-377);
(1S,2S)—N-(6-(((6-cyclopropyl-8-(3-(oxetan-3-yl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-378);
(1S,2S)—N-(6-(((6-cyclopropyl-3-fluoro-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-379);
(1S,2S)—N-(6-(((6-chloro-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-380);
(1S,2S)—N-(6-(((6-cyclopropylimidazo[1,2-a]pyridin-2-yl)methyl)amino)pyrimidin-4-yl)-2-(4-methylpyrimidin-2-yl)cyclopropane-1-carboxamide (I-381);
(1S,2S)—N-(6-(((6-cyclopropyl-8-(3-(2-(2-methoxyethoxy)ethyl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-382);
(1S,2S)—N-(6-(((6-cyclopropyl-8-(4-ethylpiperazin-1-yl)imidazo[1,2-a]pyridin-2-yl)methyl)amino)pyrimidin-4-yl)-2-(4-methylpyrimidin-2-yl)cyclopropane-1-carboxamide (I-383);
(1S,2S)—N-(6-(((6-cyclopropyl-8-(3-cyclopropyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-384);
(1S,2S)—N-(6-(((6-cyclopropyl-8-(3-ethyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-385);
rac-(1S*,2S*)—N-(6-(((6-cyclopropyl-8-(4-methyl-3,5-dioxo-1,2,4-triazolidin-1-yl)imidazo[1,2-a]pyridin-2-yl)methyl)amino)pyrimidin-4-yl)-2-(4-methylpyrimidin-2-yl)cyclopropane-1-carboxamide (I-386);
rac-(1S*,2S*)—N-(6-(((6-cyclopropyl-8-(3-methyl-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide (I-387);
rac-(1 S*,2S*)—N-(6-(((6-cyclopropyl-8-(2,4-dioxo-3-(2,2,2-trifluoroethyl)imidazolidin-1-yl)imidazo[1,2-a]pyridin-2-yl)methyl)amino)pyrimidin-4-yl)-2-(4-methylpyrimidin-2-yl)cyclopropane-1-carboxamide (I-388);
rac-(1S*,2S*)—N-(6-(((6-cyclopropyl-8-(2-oxopyrrolidin-1-yl)imidazo[1,2-a]pyridin-2-yl)methyl)amino)pyrimidin-4-yl)-2-(4-methylpyrimidin-2-yl)cyclopropane-1-carboxamide (I-389);
rac-(1S*,2S*)—N-(6-(((6-cyclopropyl-8-(2-oxooxazolidin-3-yl)imidazo[1,2-a]pyridin-2-yl)methyl)amino)pyrimidin-4-yl)-2-(4-methylpyrimidin-2-yl)cyclopropane-1-carboxamide (I-390);
rac-(1S*,2S*)—N-(6-(((6-cyclopropyl-8-(3-methyl-2,4-dioxoimidazolidin-1-yl)imidazo[1,2-a]pyridin-2-yl)methyl-d2)amino)pyrimidin-4-yl)-2-(4-methylpyrimidin-2-yl)cyclopropane-1-carboxamide (I-391);
rac-(1S*,2S*)—N-(6-(((6-cyclopropyl-8-(2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-392);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyridin-2-yl)cyclopropane-1-carboxamide (I-393);
(1S,2S)-2-(4-chloropyridin-2-yl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide (I-394);
(1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)pyrimidin-4-yl)cyclopropane-1-carboxamide (I-395);
(1S,2S)-2-(3-chlorophenyl)-N-(6-(((6-cyclopropyl-8-(2,4-dioxoimidazolidin-1-yl)imidazo[1,2-a]pyridin-2-yl)methyl)amino)pyrimidin-4-yl)cyclopropane-1-carboxamide (I-396);
(1S,2S)-2-(3-chlorophenyl)-N-(6-(((6-cyclopropyl-8-(3-(4-methoxybenzyl)-2,4-dioxoimidazolidin-1-yl)imidazo[1,2-a]pyridin-2-yl)methyl)amino)pyrimidin-4-yl)cyclopropane-1-carboxamide (I-397);
(1S,2S)-2-(3-chlorophenyl)-N-(6-(((6-cyclopropyl-8-(2,4-dioxo-3-(2,2,2-trifluoroethyl)imidazolidin-1-yl)imidazo[1,2-a]pyridin-2-yl)methyl)amino)pyrimidin-4-yl)cyclopropane-1-carboxamide (I-398);
(1S,2S)-2-(3-chlorophenyl)-N-(6-(((6-cyclopropyl-8-(3-cyclopropyl-2,4-dioxoimidazolidin-1-yl)imidazo[1,2-a]pyridin-2-yl)methyl)amino)pyrimidin-4-yl)cyclopropane-1-carboxamide (I-399);
(1S,2S)-2-(3-chlorophenyl)-N-(6-(((6-cyclopropyl-8-((3-methyl-2,4-dioxoimidazolidin-1-yl)methyl)imidazo[1,2-a]pyridin-2-yl)methyl)amino)pyrimidin-4-yl)cyclopropane-1-carboxamide (I-400);
(1S,2S)-2-(3-chlorophenyl)-N-(6-(((6-cyclopropyl-8-(4-hydroxy-1-methylpiperidin-4-yl)imidazo[1,2-a]pyridin-2-yl)methyl)amino)pyrimidin-4-yl)cyclopropane-1-carboxamide (I-401);
(1S,2S)-2-(3-chlorophenyl)-N-(6-(((6-cyclopropyl-8-(3-fluoro-1-methylazetidin-3-yl)imidazo[1,2-a]pyridin-2-yl)methyl)amino)pyrimidin-4-yl)cyclopropane-1-carboxamide (I-402);
(1S,2S)-2-(3-chlorophenyl)-N-(6-(((6-cyclopropyl-8-(4-fluoro-1-methylpiperidin-4-yl)imidazo[1,2-a]pyridin-2-yl)methyl)amino)pyrimidin-4-yl)cyclopropane-1-carboxamide (I-403);
(1S,2S)-2-(3-chlorophenyl)-N-(6-(((6-cyclopropyl-8-(4-methyl-2,5-dioxopiperazin-1-yl)imidazo[1,2-a]pyridin-2-yl)methyl)amino)pyrimidin-4-yl)cyclopropane-1-carboxamide (I-404);
rac-(1S*,2S*)-2-(3-chlorophenyl)-N-(6-(((6-cyclopropyl-8-(4H-1,2,4-triazol-4-yl)imidazo[1,2-a]pyridin-2-yl)methyl)amino)pyrimidin-4-yl)cyclopropane-1-carboxamide (I-405);
(1S,2S)—N-(6-(((6-cyclopropyl-8-(3-methyl-2,4-dioxoimidazolidin-1-yl)imidazo[1,2-a]pyridin-2-yl)methyl)(methyl)amino)-2-methoxypyrimidin-4-yl)-2-(4-methylpyrimidin-2-yl)cyclopropane-1-carboxamide (I-406);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methoxypyrimidin-4-yl)-2-(4-methylpyrimidin-2-yl)cyclopropane-1-carboxamide (I-407);
(1S,2S)—N-(2-cyclopropyl-6-(((6-cyclopropyl-8-(2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide (I-408);
(1S,2S)—N-(6-((1-(8-(3-(2-(benzyloxy)ethyl)-2,4-dioxoimidazolidin-1-yl)-6-cyclopropylimidazo[1,2-a]pyridin-2-yl)ethyl)amino)-2-cyclopropylpyrimidin-4-yl)-2-(4-methylpyrimidin-2-yl)cyclopropane-1-carboxamide (I-409);
(1S,2S)—N-(2-cyclopropyl-6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-410);
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(difluoromethyl)pyrimidin-4-yl)-2-(4-methylpyrimidin-2-yl)cyclopropane-1-carboxamide (I-411);
rac-(1S*,2S*)—N-(6-(((6-cyclopropyl-8-(3-methyl-2,4-dioxoimidazolidin-1-yl)imidazo[1,2-a]pyridin-2-yl)methyl)amino)-5-fluoropyrimidin-4-yl)-2-(4-methylpyrimidin-2-yl)cyclopropane-1-carboxamide (I-412);
rac-(1R*,2S*)-5′-chloro-N-(6-(((6-cyclopropylimidazo[1,2-a]pyridin-2-yl)methyl)amino)pyrimidin-4-yl)-1′-methyl-2′-oxospiro[cyclopropane-1,3′-indoline]-2-carboxamide (I-413);
rac-(1S*,2S*)—N-(4-(((6-cyclopropylimidazo[1,2-a]pyridin-2-yl)methyl)amino)pyridin-2-yl)-2-(4-methylpyrimidin-2-yl)cyclopropane-1-carboxamide (I-414);
(1S,2S)—N-(6-(((R)-1-(6-methoxy-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-415);
(1S,2S)—N-(6-(((S)-1-(6-(difluoromethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-416);
(1S,2S)—N-(6-(((R)-1-(6-(difluoromethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-417);
(1S,2S)—N-(6-(((R)-1-(6-cyclopropyl-8-(2-oxopyrrolidin-1-yl)imidazo[1,2-a]pyridin-2-yl)ethyl)amino)pyrimidin-4-yl)-2-(4-methylpyrimidin-2-yl)cyclopropane-1-carboxamide (I-418);
(1S,2S)—N-(6-(((R)-1-(6-ethyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-419);
(1S,2S)—N-(6-(((R)-1-(6-cyclopropyl-8-(7-methyl-6,8-dioxo-2-oxa-5,7-diazaspiro[3.4]octan-5-yl)imidazo[1,2-a]pyridin-2-yl)ethyl)amino)pyrimidin-4-yl)-2-(4-methylpyrimidin-2-yl)cyclopropane-1-carboxamide (I-420);
(1S,2S)—N-(6-(((R)-1-(6-chloro-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-421);
(1S,2S)—N-(6-(((R)-1-(6-cyclopropyl-8-((dimethyl(oxo)-16-sulfaneylidene)amino)imidazo[1,2-a]pyridin-2-yl)ethyl)amino)pyrimidin-4-yl)-2-(4-methylpyrimidin-2-yl)cyclopropane-1-carboxamide (I-422);
(1S,2S)—N-(6-(((R)-1-(6-methyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-423);
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-424);
(3S,5R)-5-(6-cyclopropyl-8-(3-methyl-2,4-dioxoimidazolidin-1-yl)imidazo[1,2-a]pyridin-2-yl)-1-(6-((1S,2S)-2-(4-methylpyrimidin-2-yl)cyclopropane-1-carboxamido)pyrimidin-4-yl)pyrrolidin-3-yl propionate (I-425);
(1S,2S)—N-(6-((2R)-2-(6-cyclopropylimidazo[1,2-a]pyridin-2-yl)-4-hydroxy-4-methylpyrrolidin-1-yl)pyrimidin-4-yl)-2-(4-methylpyrimidin-2-yl)cyclopropane-1-carboxamide (I-426);
(1S,2S)—N-(6-(((S)-1-(6-(difluoromethyl)-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-427);
(1S,2S)—N-(6-(((R)-1-(6-(difluoromethyl)-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-428);
(1S,2S)—N-(6-(((R)-1-(6-ethyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-429);
(1S,2S)—N-(2-methyl-6-(((1R)-1-(6-methyl-8-(2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide (I-430);
(1S,2S)—N-(6-(((R)-1-(6-cyclopropyl-8-((dimethyl(oxo)-16-sulfaneylidene)amino)imidazo[1,2-a]pyridin-2-yl)ethyl)amino)-2-methylpyrimidin-4-yl)-2-(4-methylpyrimidin-2-yl)cyclopropane-1-carboxamide (I-431);
(1S,2S)—N-(6-(((R)-1-(6-cyclopropyl-8-(3,5,5-trimethyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-432);
(1S,2S)—N-(6-(((1R)-1-(6-cyclopropyl-8-(2-oxo-3-azabicyclo[3.1.0]hexan-3-yl)imidazo[1,2-a]pyridin-2-yl)ethyl)amino)-2-methylpyrimidin-4-yl)-2-(4-methylpyrimidin-2-yl)cyclopropane-1-carboxamide (I-433);
(1S,2S)—N-(2-methyl-6-(((R)-1-(6-methyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-434);
(1S,2S)—N-(6-(((R)-1-(6-cyclopropyl-8-(5-methyl-6-oxo-2-oxa-5,7-diazaspiro[3.4]octan-7-yl)imidazo[1,2-a]pyridin-2-yl)ethyl)amino)-2-methylpyrimidin-4-yl)-2-(4-methylpyrimidin-2-yl)cyclopropane-1-carboxamide (I-435);
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-436);
(1S,2S)—N-(6-(((R)-1-(6-cyclopropyl-8-(6-oxo-2,5-dioxa-7-azaspiro[3.4]octan-7-yl)imidazo[1,2-a]pyridin-2-yl)ethyl)amino)-2-methylpyrimidin-4-yl)-2-(4-methylpyrimidin-2-yl)cyclopropane-1-carboxamide (I-437);
(1S,2S)—N-(2-(methoxymethyl)-6-(((R)-1-(6-methyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-438);
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)-2-(methoxymethyl)pyrimidin-4-yl)-2-(4-methylpyrimidin-2-yl)cyclopropane-1-carboxamide (I-439);
(1S,2S)—N-(2-(((R)-1-(6-cyclopropyl-8-(3-methyl-2,4-dioxoimidazolidin-1-yl)imidazo[1,2-a]pyridin-2-yl)ethyl)amino)-6-methylpyrimidin-4-yl)-2-(4-methylpyrimidin-2-yl)cyclopropane-1-carboxamide (I-440);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(dimethylamino)pyrimidin-4-yl)-2-(4-methylpyrimidin-2-yl)cyclopropane-1-carboxamide (I-441);
(1S,2S)-2-(4-chloropyridin-2-yl)-N-(6-((2R,4S)-2-(6-cyclopropylimidazo[1,2-a]pyridin-2-yl)-4-(hydroxymethyl)pyrrolidin-1-yl)pyrimidin-4-yl)cyclopropane-1-carboxamide (I-442);
(1S,2S)—N-(6-(((R)-1-(6-cyclopropyl-8-((1R*,5S*)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide (I-443);
(1S,2S)—N-(6-(((R)-1-(6-cyclopropyl-8-((1R*,5S*)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide,
first eluting isomer (I-444);
(1S,2S)—N-(6-(((R)-1-(6-cyclopropyl-8-((1R*,5S*)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide,
second eluting isomer (I-445);
(1S,2S)—N-(6-(((R)-1-(6-cyclopropyl-8-((1S,5R)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-((1R,5S)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide (I-446);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide, first eluting isomer (I-447);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide, second eluting isomer (I-448);
(1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide;
(1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide;
N-(6-((1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
rac-(1S*,2S*)—N-(6-((1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-449);
rac-(1S*,2S*)—N-(6-((1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, first eluting isomer (I-450);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-451);
(1S,2S)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-452);
rac-(1S*,2S*)—N-(6-((1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, fourth eluting isomer (I-453);
(1R,2R)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1R,2R)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide,
N-(6-((1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-((1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-454);
(1S,2S)—N-(6-(((S)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide, first eluting isomer (I-455);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide,
second eluting isomer (I-456);
rac-(1S,2S)-2-(3-chlorophenyl)-N-(6-(((6-cyclopropyl-8-((1R*,5S*)-2-oxo-3-azabicyclo[3.1.0]hexan-3-yl)imidazo[1,2-a]pyridin-2-yl)methyl)amino)pyrimidin-4-yl)cyclopropane-1-carboxamide (I-457);
rac-(1S,2S)-2-(3-chlorophenyl)-N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)cyclopropane-1-carboxamide, first eluting isomer (I-458);
rac-(1S,2S)-2-(3-chlorophenyl)-N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)cyclopropane-1-carboxamide, second eluting isomer (I-459);
(1S,2S)-2-(3-chlorophenyl)-N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)cyclopropane-1-carboxamide;
(1S,2S)—N-(5-((6-cyclopropyl-8-((1S,5R)-2-oxo-3-azabicyclo[3.1.0]hexan-3-yl)imidazo[1,2-a]pyridin-2-yl)methoxy)pyridazin-3-yl)-2-(4-methylpyrimidin-2-yl)cyclopropane-1-carboxamide;
(1S,2S)—N-(2-cyclopropyl-6-((1-(6-cyclopropyl-8-(3-(2-hydroxyethyl)-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-460);
N-((6-cyclopropyl-8-(3-methyl-2,4-dioxoimidazolidin-1-yl)imidazo[1,2-a]pyridin-2-yl)methyl)-N-(6-((1S,2S)-2-(4-methylpyrimidin-2-yl)cyclopropane-1-carboxamido)pyrimidin-4-yl)glycine (I-461);
(1S,2S)—N-(6-(((6-cyclopropyl-8-(3-(2-hydroxyethyl)-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide (I-462);
(1S,2S)-2-(3-chlorophenyl)-N-(6-(((6-cyclopropyl-8-(3-(2-hydroxyethyl)-2,4-dioxoimidazolidin-1-yl)imidazo[1,2-a]pyridin-2-yl)methyl)amino)pyrimidin-4-yl)cyclopropane-1-carboxamide (I-463);
(1S,2S)—N-(6-(((6-cyclopropyl-8-(3-(2-hydroxyethyl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-464);
2-(3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)acetic acid. (I-465);
(3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl dihydrogen phosphate (I-466);
1-((((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamoyl)oxy)ethyl isobutyrate (I-467);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide (I-468);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(hydroxymethyl)pyrimidin-4-yl)-2-(4-methylpyrimidin-2-yl)cyclopropane-1-carboxamide (I-469);
(1S,2S)—N-(6-((2R,4S)-4-hydroxy-2-(6-((1S*,2S*)-2-methylcyclopropyl)imidazo[1,2-a]pyridin-2-yl)pyrrolidin-1-yl)pyrimidin-4-yl)-2-(4-methylpyrimidin-2-yl)cyclopropane-1-carboxamide (I-470);
rac-(1S*,2S*)—N-(6-((2R,4S)-2-(6-cyclopropyl-8-(2-oxooxazolidin-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (I-471);
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(4-methylpyridin-2-yl)cyclopropane-1-carboxamide (I-472);
rac-(1S*,2S*)—N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (I-473);
rac-(1S*,2S*)—N-(6-((2R,4S)-2-(6-cyclopropyl-8-(2,4-dioxoimidazolidin-1-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (I-474);
(1S,2S)-2-(4-chloropyridin-2-yl)-N-(6-((2R,4S)-2-(6-cyclopropyl-8-(2,4-dioxoimidazolidin-1-yl)imidazo[1,2-a]pyridin-2-yl)-4-hydroxypyrrolidin-1-yl)pyrimidin-4-yl)cyclopropane-1-carboxamide (I-475);
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(4-methylpyrimidin-2-yl)cyclopropane-1-carboxamide (I-476);
rac-(1S*,2S*)-2-(5-chloro-2-cyanophenyl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide (I-477);
(1S,2S)-2-(4-chloropyridin-2-yl)-N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)cyclopropane-1-carboxamide (I-478);
(1S,2S)-2-(3-chlorophenyl)-N-(6-((2R,4S)-2-(6-cyclopropyl-8-(2-oxooxazolidin-3-yl)imidazo[1,2-a]pyridin-2-yl)-4-hydroxypyrrolidin-1-yl)pyrimidin-4-yl)cyclopropane-1-carboxamide (I-479);
(1S,2S)-2-(3-chlorophenyl)-N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)cyclopropane-1-carboxamide (I-480);
rac-(1S*,2S*)-2-(4-chloropyrimidin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide (I-481);
rac-(1S*,2S*)—N-(6-((2R,4S)-2-(6-cyclopropyl-8-(2-oxo-3-azabicyclo[3.1.0]hexan-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (I-482);
(1S,2S)-2-(3-chlorophenyl)-N-(6-((2S,3S,4R)-2-(6-cyclopropylimidazo[1,2-a]pyridin-2-yl)-3,4-dihydroxypyrrolidin-1-yl)pyrimidin-4-yl)cyclopropane-1-carboxamide (I-483);
(1S,2S)-2-(3-chlorophenyl)-N-(6-((2R,3S)-2-(6-cyclopropylimidazo[1,2-a]pyridin-2-yl)-3-hydroxypyrrolidin-1-yl)pyrimidin-4-yl)cyclopropane-1-carboxamide (I-484);
(1S,2S)-2-(3-chlorophenyl)-N-(6-((2S,3R)-2-(6-cyclopropylimidazo[1,2-a]pyridin-2-yl)-3-hydroxypyrrolidin-1-yl)pyrimidin-4-yl)cyclopropane-1-carboxamide (I-485);
(1S,2S)—N-(6-((2R,4S)-2-(6-cyclopropylimidazo[1,2-a]pyridin-2-yl)-4-hydroxypyrrolidin-1-yl)pyrimidin-4-yl)-2-(3-ethynylphenyl)cyclopropane-1-carboxamide (I-486);
(1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)-2-hydroxypyrimidin-4-yl)cyclopropane-1-carboxamide (I-487);
(1S,2S)-2-(3-chlorophenyl)-N-(4-(((6-cyclopropyl-8-(4-hydroxy-1-methylpiperidin-4-yl)imidazo[1,2-a]pyridin-2-yl)methyl)amino)pyridin-2-yl)cyclopropane-1-carboxamide (I-488);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide (I-489);
rac-(1S*,2S*)—N-(6-(((6-cyclopropyl-8-(3-methyl-2,4-dioxoimidazolidin-1-yl)imidazo[1,2-a]pyridin-2-yl)methyl)(methyl)amino)-2-methylpyrimidin-4-yl)-2-fluoro-2-(4-methylpyrimidin-2-yl)cyclopropane-1-carboxamide (I-490);
rac-(-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(5-fluoro-4-methylpyrimidin-2-yl)cyclopropane-1-carboxamide (I-491);
rac-(-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(5-fluoro-4-methylpyrimidin-2-yl)cyclopropane-1-carboxamide (I-492);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(2-methylthiazol-4-yl)cyclopropane-1-carboxamide (I-493);
rac-(1S*,3S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluoro-3-(4-methylpyrimidin-2-yl)cyclopropane-1-carboxamide (I-494);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methyl-1,3,5-triazin-2-yl)cyclopropane-1-carboxamide (I-495);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluoro-2-(4-methylpyrimidin-2-yl)cyclopropane-1-carboxamide (I-496);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4,6-dimethylpyrimidin-2-yl)cyclopropane-1-carboxamide (I-497);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(6-methylpyrazin-2-yl)cyclopropane-1-carboxamide (I-498);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(2-methylpyrimidin-4-yl)cyclopropane-1-carboxamide (I-499);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(6-methylpyridin-2-yl)cyclopropane-1-carboxamide (I-500);
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(trifluoromethyl)pyrimidin-4-yl)-2-(4-methylpyrimidin-2-yl)cyclopropane-1-carboxamide (I-501);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(3-fluoro-6-methylpyridin-2-yl)cyclopropane-1-carboxamide (I-502);
rac-(1S*,3S*)-3-(3-chlorophenyl)-N-(6-(((6-cyclopropylimidazo[1,2-a]pyridin-2-yl)methyl)amino)pyrimidin-4-yl)-2,2-difluorocyclopropane-1-carboxamide (I-503);
rac-(1S*,2S*)-2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)cyclopropane-1-carboxamide (I-504);
(1R*,2R*,3S*)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide, isomer 2 (I-505);
(1R*,2R*,3S*)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide, isomer 3 (I-506);
(1R*,2R*,3S*)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide, isomer 1 (I-507);
(1R*,2R*,3S*)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide, isomer 4 (I-508);
(1R,2R,3S)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide;
(1R,2R,3R)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide;
(1S,2S,3S)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide;
(1S,2S,3R)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide;
rac-(1R*,2S*)-5′-chloro-N-(6-(((6-cyclopropylimidazo[1,2-a]pyridin-2-yl)methyl)amino)pyrimidin-4-yl)-2′-oxospiro[cyclopropane-1,3′-indoline]-2-carboxamide (I-509);
rac-(1R*,2R*)-5′-chloro-N-(6-(((6-cyclopropylimidazo[1,2-a]pyridin-2-yl)methyl)amino)pyrimidin-4-yl)-2′-oxospiro[cyclopropane-1,3′-indoline]-2-carboxamide (I-510);
rac-(1R*,2R*,3S*)-2-(3-chlorophenyl)-N-(6-(((6-cyclopropylimidazo[1,2-a]pyridin-2-yl)methyl)amino)pyrimidin-4-yl)-3-fluorocyclopropane-1-carboxamide (I-511);
(1S,2S)-2-(3-chlorophenyl)-N-(4-(((6-cyclopropyl-8-(2,4-dioxo-3-(2,2,2-trifluoroethyl)imidazolidin-1-yl)imidazo[1,2-a]pyridin-2-yl)methyl)amino)pyridin-2-yl)cyclopropane-1-carboxamide (I-512);
(1S,2S)-2-(3-chlorophenyl)-N-(4-(((6-cyclopropyl-8-(3-cyclopropyl-2,4-dioxoimidazolidin-1-yl)imidazo[1,2-a]pyridin-2-yl)methyl)amino)pyridin-2-yl)cyclopropane-1-carboxamide (I-513);
rac-(1R*,2S*)-5′-chloro-N-(4-(((6-cyclopropylimidazo[1,2-a]pyridin-2-yl)methyl)amino)pyridin-2-yl)-2′-oxospiro[cyclopropane-1,3′-indoline]-2-carboxamide (I-514);
rac-(1S*,2S*)—N-(4-(((8-(3-(2-(benzyloxy)ethyl)-2,4-dioxoimidazolidin-1-yl)-6-cyclopropylimidazo[1,2-a]pyridin-2-yl)methyl)amino)-5-fluoropyrimidin-2-yl)-2-(4-methylpyrimidin-2-yl)cyclopropane-1-carboxamide (I-515);
rac-(1S*,2S*)—N-(4-(((6-cyclopropyl-8-(2,4-dioxoimidazolidin-1-yl)imidazo[1,2-a]pyridin-2-yl)methyl)(methyl)amino)-5-fluoropyrimidin-2-yl)-2-(4-methylpyrimidin-2-yl)cyclopropane-1-carboxamide (I-516);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide (I-517);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(trifluoromethyl)pyrimidin-2-yl)-2-(4-methylpyrimidin-2-yl)cyclopropane-1-carboxamide (I-518);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-methylpyrimidin-2-yl)-2-(4-methylpyrimidin-2-yl)cyclopropane-1-carboxamide (I-519);
rac-(1S*,2S*)—N-(4-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide (I-520);
rac-(1S*,2S*)-2-(4-chloropyridin-2-yl)-N-(4-(((6-cyclopropyl-8-(2,4-dioxoimidazolidin-1-yl)imidazo[1,2-a]pyridin-2-yl)methyl)amino)pyrimidin-2-yl)cyclopropane-1-carboxamide (I-521);
rac-(1S*,2S*)—N-(4-cyclopropyl-6-(((6-cyclopropyl-8-(2,4-dioxoimidazolidin-1-yl)imidazo[1,2-a]pyridin-2-yl)methyl)(methyl)amino)-1,3,5-triazin-2-yl)-2-(4-methylpyrimidin-2-yl)cyclopropane-1-carboxamide (I-522);
rac-(1S*,2S*)—N-(4-cyclopropyl-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-523);
rac-(1S*,2S*)—N-(4-(((6-cyclopropyl-8-(2,4-dioxoimidazolidin-1-yl)imidazo[1,2-a]pyridin-2-yl)methyl)(methyl)amino)-6-methyl-1,3,5-triazin-2-yl)-2-(4-methylpyrimidin-2-yl)cyclopropane-1-carboxamide (I-524);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-methyl-1,3,5-triazin-2-yl)-2-(4-methylpyrimidin-2-yl)cyclopropane-1-carboxamide (I-525);
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrazin-2-yl)-2-(4-methylpyrimidin-2-yl)cyclopropane-1-carboxamide (I-526);
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(5-methyl-1,2,4-thiadiazol-3-yl)cyclopropane-1-carboxamide (I-527);
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-1-fluoro-2-(4-methylpyrimidin-2-yl)cyclopropane-1-carboxamide (I-528);
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methoxypyrimidin-2-yl)cyclopropane-1-carboxamide (I-529);
(1S,2S)—N-(6-(((1R)-1-(6-(1-fluoroethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-530);
(1S,2S)—N-(6-(((R)-1-(6-(1,1-difluoroethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-531);
(1S,2S)—N-(6-(((R)-1-(6-cyclopropyl-8-(2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-532);
(1S,2S)—N-(6-(((R)-1-(6-cyclopropyl-8-(2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-533);
(1S,2S)—N-(6-(((R)-1-(6-cyclopropyl-8-(2-oxooxazolidin-3-yl)imidazo[1,2-a]pyridin-2-yl)ethyl)amino)pyrimidin-4-yl)-2-(4-methylpyrimidin-2-yl)cyclopropane-1-carboxamide (I-534);
(1S,2S)—N-(6-(((6-(2,2-difluorocyclopropyl)-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-535);
(1S,2S)—N-(6-(((R)-1-(6-cyclopropyl-8-(3-methyl-2-oxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-536);
(1S,2S)—N-(6-(((6-ethyl-8-(3-methyl-2,4-dioxoimidazolidin-1-yl)imidazo[1,2-a]pyridin-2-yl)methyl)amino)-2-methylpyrimidin-4-yl)-2-(4-methylpyrimidin-2-yl)cyclopropane-1-carboxamide (I-537);
(1S,2S)—N-(6-(((6-ethyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-538);
rac-(1S*,2S*)—N-(4-cyclopropyl-6-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide (I-539);
rac-(1S*,2S*)—N-(4-(((6-cyclopropyl-8-(3-(2-hydroxyethyl)-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide (I-540);
rac-(1S*,2S*)—N-(4-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-2-yl)-2-(4-methylpyrimidin-2-yl)cyclopropane-1-carboxamide (I-541);
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(3-fluoro-4-methylpyridin-2-yl)cyclopropane-1-carboxamide (I-542);
rac-(1S*,3S*)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide (I-543);
rac-(1S*,3S*)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide,
first eluting isomer (I-544);
rac-(1S*,3S*)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide,
second eluting isomer (I-545);
(1R,3R)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide;
(1S,3S)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide;
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide (I-546);
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide, first eluting isomer (I-547);
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide, second eluting isomer (I-548);
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide;
(1R,2R)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide;
(1S,2S)—N-(6-(1-(6-cyclopropyl-8-(3-methyl-2,4-dioxoimidazolidin-1-yl)imidazo[1,2-a]pyridin-2-yl)ethoxy)pyrimidin-4-yl)-2-(4-methylpyrimidin-2-yl)cyclopropane-1-carboxamide (I-549);
methyl 2-(3-(6-cyclopropyl-2-(((2-methyl-6-((1S,2S)-2-(4-methylpyrimidin-2-yl)cyclopropane-1-carboxamido)pyrimidin-4-yl)oxy)methyl)imidazo[1,2-a]pyridin-8-yl)-2,5-dioxoimidazolidin-1-yl)acetate (I-550);
(1S,2S)—N-(6-((6-cyclopropyl-8-(3-methyl-2,4-dioxoimidazolidin-1-yl)imidazo[1,2-a]pyridin-2-yl)methoxy)-2-(methoxymethyl)pyrimidin-4-yl)-2-(4-methylpyrimidin-2-yl)cyclopropane-1-carboxamide (I-551);
(1S,2S)—N-(6-(1-(6-cyclopropyl-8-(3-methyl-2,4-dioxoimidazolidin-1-yl)imidazo[1,2-a]pyridin-2-yl)ethoxy)-2-methylpyrimidin-4-yl)-2-(4-methylpyrimidin-2-yl)cyclopropane-1-carboxamide (I-552);
(1S,2S)—N-(6-((6-cyclopropyl-8-(6-oxo-2,5-dioxa-7-azaspiro[3.4]octan-7-yl)imidazo[1,2-a]pyridin-2-yl)methoxy)-2-methylpyrimidin-4-yl)-2-(4-methylpyrimidin-2-yl)cyclopropane-1-carboxamide (I-553);
(1S,2S)—N-(6-((7-cyclopropyl-5-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-554);
(1S,2S)—N-(6-((6-cyclopropyl-8-(3-methyl-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide (I-555);
(1S,2S)—N-(6-((6-cyclopropyl-8-(3-methyl-2,4-dioxoimidazolidin-1-yl)imidazo[1,2-a]pyridin-2-yl)methoxy)-2-methoxypyrimidin-4-yl)-2-(4-methylpyrimidin-2-yl)cyclopropane-1-carboxamide (I-556);
(1S,2S)-2-(3-chlorophenyl)-N-(5-((6-cyclopropyl-8-(3-methyl-2,4-dioxoimidazolidin-1-yl)imidazo[1,2-a]pyridin-2-yl)methoxy)pyridazin-3-yl)cyclopropane-1-carboxamide (I-557);
(1S,2S)—N-(5-((6-cyclopropyl-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (I-558);
rac-(1S*,2S*)-2-(2-cyano-5-methoxyphenyl)-N-(5-((6-cyclopropyl-8-(3-methyl-2,4-dioxoimidazolidin-1-yl)imidazo[1,2-a]pyridin-2-yl)methoxy)pyridazin-3-yl)cyclopropane-1-carboxamide (I-559);
(1S,2S)—N-(5-((6-cyclopropyl-8-(3-methyl-2,4-dioxoimidazolidin-1-yl)imidazo[1,2-a]pyridin-2-yl)methoxy)-6-methylpyridazin-3-yl)-2-(4-methylpyrimidin-2-yl)cyclopropane-1-carboxamide (I-560);
(1S,2S)—N-(5-((6-cyclopropyl-8-(methylsulfonyl)imidazo[1,2-a]pyridin-2-yl)methoxy)pyridazin-3-yl)-2-(4-methylpyrimidin-2-yl)cyclopropane-1-carboxamide (I-561);
(1S,2S)—N-(6-((6-cyclopropyl-8-(3-(2-hydroxy-2-methylpropyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide (I-562);
(1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide (I-563);
(1S,2S)—N-(6-((6-cyclopropyl-8-(3-(2-(dimethylamino)ethyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide (I-564);
(1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide (I-565);
rac-(1S*,2S*)-2-(3-chlorophenyl)-N-(4-((8-(2-cyanoethyl)-6-cyclopropylimidazo[1,2-a]pyridin-2-yl)methoxy)pyridin-2-yl)cyclopropane-1-carboxamide (I-566);
rac-(1S*,2S*)-2-(3-chlorophenyl)-N-(4-((8-cyano-6-cyclopropylimidazo[1,2-a]pyridin-2-yl)methoxy)pyridin-2-yl)cyclopropane-1-carboxamide (I-567);
(1S,2S)—N-(5-((6-cyclopropylimidazo[1,2-a]pyridin-2-yl)methoxy)pyridazin-3-yl)-2-(4-methylpyrimidin-2-yl)cyclopropane-1-carboxamide (I-568);
(1S,2S)-2-(3-chlorophenyl)-N-(3-((6-cyclopropylimidazo[1,2-a]pyridin-2-yl)methoxy)pyridin-2-yl)cyclopropane-1-carboxamide (I-569);
(1S,2S)-2-(3-chlorophenyl)-N-(3-((6-cyclopropylimidazo[1,2-a]pyridin-2-yl)methoxy)pyrazin-2-yl)cyclopropane-1-carboxamide (I-570);
(1S,2S)—N-(5-((6-cyclopropyl-8-(2-oxo-3-azabicyclo[3.1.0]hexan-3-yl)imidazo[1,2-a]pyridin-2-yl)methoxy)pyridazin-3-yl)-2-(4-methylpyrimidin-2-yl)cyclopropane-1-carboxamide (I-571);
rac-(1S*,2S*)—N-(4-((8-(aminomethyl)-6-cyclopropylimidazo[1,2-a]pyridin-2-yl)methoxy)pyridin-2-yl)-2-(3-chlorophenyl)cyclopropane-1-carboxamide (I-572);
(1S,2S)—N-(4-((6-cyclopropyl-8-(3-methyl-2,4-dioxoimidazolidin-1-yl)imidazo[1,2-a]pyridin-2-yl)methoxy)-6-methyl-1,3,5-triazin-2-yl)-2-(4-methylpyrimidin-2-yl)cyclopropane-1-carboxamide (I-573);
rac-(1S*,2S*)-2-(3-chlorophenyl)-N-(5-(((6-cyclopropylimidazo[1,2-a]pyridin-2-yl)methyl)amino)pyridin-3-yl)cyclopropane-1-carboxamide (I-574);
rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridin-3-yl)-2-(4-methylpyrimidin-2-yl)cyclopropane-1-carboxamide (I-575);
rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[4,3-c]pyridin-4-yl)-2-(4-methylpyrimidin-2-yl)cyclopropane-1-carboxamide (I-576);
(1S,2S)-2-(4-chloropyridin-2-yl)-N-(2-((6-cyclopropylimidazo[1,2-a]pyridin-2-yl)methyl)-2H-pyrazolo[4,3-c]pyridin-4-yl)cyclopropane-1-carboxamide (I-577);
rac-(1S*,2S*)-2-(5-chloro-2-nitrophenyl)-N-(2-((6-cyclopropylimidazo[1,2-a]pyridin-2-yl)methyl)-2H-pyrazolo[4,3-c]pyridin-4-yl)cyclopropane-1-carboxamide (I-578);
rac-(1S*,2S*)-2-(5-chloro-2-cyanophenyl)-N-(2-((6-cyclopropylimidazo[1,2-a]pyridin-2-yl)methyl)-2H-pyrazolo[4,3-c]pyridin-4-yl)cyclopropane-1-carboxamide (I-579);
(1S,2S)-2-(3-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide (I-580);
(1S,2S)-2-(3-chlorophenyl)-N-(2-((6-cyclopropylimidazo[1,2-a]pyridin-2-yl)methyl)-6-methyl-2H-pyrazolo[4,3-c]pyridin-4-yl)cyclopropane-1-carboxamide (I-581);
(1S,2S)—N-(5-((6-cyclopropyl-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (I-582);
(1S,2S)-2-(3-chlorophenyl)-N-(2-((6-cyclopropylimidazo[1,2-a]pyridin-2-yl)methyl)-2H-[1,2,3]triazolo[4,5-c]pyridin-4-yl)cyclopropane-1-carboxamide (I-583);
(1S,2S)-2-(3-chlorophenyl)-N-(1-((6-cyclopropylimidazo[1,2-a]pyridin-2-yl)methyl)-1H-pyrazolo[4,3-c]pyridin-4-yl)cyclopropane-1-carboxamide (I-584);
rac-(1S*,2S*)-2-(2-amino-5-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide (I-585);
rac-(1S*,2S*)-2-(5-chloro-2-(1H-tetrazol-1-yl)phenyl)-N-(2-((6-cyclopropylimidazo[1,2-a]pyridin-2-yl)methyl)-2H-pyrazolo[4,3-c]pyridin-4-yl)cyclopropane-1-carboxamide (I-586);
rac-(1S*,2S*)-2-(3-chlorophenyl)-N-(6-((6-cyclopropylimidazo[1,2-a]pyridin-2-yl)methyl)pyrimidin-4-yl)cyclopropane-1-carboxamide (I-587);
rac-(1S*,2S*)-2-(3-chlorophenyl)-N-((1-((6-cyclopropylimidazo[1,2-a]pyridin-2-yl)methyl)-1H-1,2,3-triazol-4-yl)methyl)cyclopropane-1-carboxamide (I-588);
rac-(1S*,2S*)-2-(3-chlorophenyl)-N—((S*)1-(1-((6-cyclopropylimidazo[1,2-a]pyridin-2-yl)methyl)-1H-1,2,3-triazol-4-yl)ethyl)cyclopropane-1-carboxamide (I-589), Isomer 1;
rac-(1R*,2R*)-2-(3-chlorophenyl)-N—((S*)1-(1-((6-cyclopropylimidazo[1,2-a]pyridin-2-yl)methyl)-1H-1,2,3-triazol-4-yl)ethyl)cyclopropane-1-carboxamide (I-590), Isomer 2;
(1S*,2S*)-2-(3-chlorophenyl)-N—((S)-1-(1-((6-cyclopropylimidazo[1,2-a]pyridin-2-yl)methyl)-1H-1,2,3-triazol-4-yl)ethyl)cyclopropane-1-carboxamide;
(1S,2S)-2-(3-chlorophenyl)-N—((S)-1-(1-((6-cyclopropylimidazo[1,2-a]pyridin-2-yl)methyl)-1H-1,2,3-triazol-4-yl)ethyl)cyclopropane-1-carboxamide;
(1R,2R)-2-(3-chlorophenyl)-N—((S)-1-(1-((6-cyclopropylimidazo[1,2-a]pyridin-2-yl)methyl)-1H-1,2,3-triazol-4-yl)ethyl)cyclopropane-1-carboxamide;
(1S*,2S*)-2-(3-chlorophenyl)-N—((R)-1-(1-((6-cyclopropylimidazo[1,2-a]pyridin-2-yl)methyl)-1H-1,2,3-triazol-4-yl)ethyl)cyclopropane-1-carboxamide;
(1S,2S)-2-(3-chlorophenyl)-N—((R)-1-(1-((6-cyclopropylimidazo[1,2-a]pyridin-2-yl)methyl)-1H-1,2,3-triazol-4-yl)ethyl)cyclopropane-1-carboxamide;
(1R,2R)-2-(3-chlorophenyl)-N—((R)-1-(1-((6-cyclopropylimidazo[1,2-a]pyridin-2-yl)methyl)-1H-1,2,3-triazol-4-yl)ethyl)cyclopropane-1-carboxamide;
(1S,2S)-2-(3-chlorophenyl)-N-((1-((6-cyclopropyl-8-(4H-1,2,4-triazol-4-yl)imidazo[1,2-a]pyridin-2-yl)methyl)-1H-1,2,3-triazol-4-yl)methyl)cyclopropane-1-carboxamide (I-591);
rac-(1S*,2S*)-2-(3-chlorophenyl)-N-(2-((6-cyclopropylimidazo[1,2-a]pyridin-2-yl)methyl)-2H-pyrazolo[3,4-c]pyridin-7-yl)cyclopropane-1-carboxamide (I-592);
rac-(1S*,2S*)-2-(3-chlorophenyl)-N-(1-((6-cyclopropylimidazo[1,2-a]pyridin-2-yl)methyl)-1H-1,2,3-triazol-4-yl)cyclopropane-1-carboxamide (I-593);
rac-(1S*,2S*)-2-(3-chlorophenyl)-N-(1-((6-cyclopropylimidazo[1,2-a]pyridin-2-yl)methyl)-1H-imidazo[4,5-c]pyridin-6-yl)cyclopropane-1-carboxamide (I-594);
(1S,2S)-2-(3-chlorophenyl)-N-(4-(((6-cyclopropyl-8-((methylamino)methyl)imidazo[1,2-a]pyridin-2-yl)methyl)amino)pyridin-2-yl)cyclopropane-1-carboxamide (I-595);
(1S,2S)-2-(3-chlorophenyl)-N-(5-((6-cyclopropylimidazo[1,2-a]pyridin-2-yl)methoxy)pyrimidin-4-yl)cyclopropane-1-carboxamide (I-596);
(1S,2S)-2-(3-chlorophenyl)-N-(1-((6-cyclopropylimidazo[1,2-a]pyridin-2-yl)methyl)-2,3-dihydro-1H-pyrido[3,4-b][1,4]oxazin-7-yl)cyclopropane-1-carboxamide (I-597);
(1S,2S)-2-(3-chlorophenyl)-N-(4-(((6-cyclopropyl-8-(2-(dimethylamino)ethyl)imidazo[1,2-a]pyridin-2-yl)methyl)amino)pyridin-2-yl)cyclopropane-1-carboxamide (I-598);
(1S,2S)-2-(3-chlorophenyl)-N-(4-(((6-cyclopropyl-8-(4-(2-methoxyethyl)piperazin-1-yl)imidazo[1,2-a]pyridin-2-yl)methyl)amino)pyridin-2-yl)cyclopropane-1-carboxamide (I-599);
ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (I-600);
(1S,2S)-2-(3-chlorophenyl)-N-(4-(((6-cyclopropyl-8-(4-(cyclopropylmethyl)piperazin-1-yl)imidazo[1,2-a]pyridin-2-yl)methyl)amino)pyridin-2-yl)cyclopropane-1-carboxamide (I-601);
2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetic acid (I-602);
(1S,2S)-2-(3-chlorophenyl)-N-(4-(((6-cyclopropyl-8-(4-(2-hydroxyethyl)piperazin-1-yl)imidazo[1,2-a]pyridin-2-yl)methyl)amino)pyridin-2-yl)cyclopropane-1-carboxamide (I-603);
(1S,2S)-2-(3-chlorophenyl)-N-(4-(((6-cyclopropylimidazo[1,2-a]pyridin-2-yl)methyl)(2-hydroxyethyl)amino)pyridin-2-yl)cyclopropane-1-carboxamide (I-604);
(1S,2S)-2-(3-chlorophenyl)-N-(4-(((6-cyclopropyl-8-(4-methylpiperazin-1-yl)imidazo[1,2-a]pyridin-2-yl)methyl)amino)-6-methylpyridin-2-yl)cyclopropane-1-carboxamide (I-605);
(1S,2S)-2-(3-chlorophenyl)-N-(5-(((6-cyclopropylimidazo[1,2-a]pyridin-2-yl)methyl)amino)-1,3,4-thiadiazol-2-yl)cyclopropane-1-carboxamide (I-606);
(1S,2S)-2-(3-chlorophenyl)-N-(4-(((6-cyclopropyl-8-(2-(methylamino)ethyl)imidazo[1,2-a]pyridin-2-yl)methyl)amino)pyridin-2-yl)cyclopropane-1-carboxamide (I-607);
(1S,2S)-2-(3-chlorophenyl)-N-(4-(((6-cyclopropyl-8-(3-hydroxy-1-methylazetidin-3-yl)imidazo[1,2-a]pyridin-2-yl)methyl)amino)pyridin-2-yl)cyclopropane-1-carboxamide (I-608);
(1S,2S)-2-(3-chlorophenyl)-N-(4-(((6-cyclopropyl-8-(1-methylazetidin-3-yl)imidazo[1,2-a]pyridin-2-yl)methyl)amino)pyridin-2-yl)cyclopropane-1-carboxamide (I-609);
(1S,2S)-2-(3-chlorophenyl)-N-(5-(((6-cyclopropylimidazo[1,2-a]pyridin-2-yl)methyl)amino)-1,2,4-thiadiazol-3-yl)cyclopropane-1-carboxamide (I-610);
(1S,2S)-2-(3-chlorophenyl)-N-(4-(((6-cyclopropylimidazo[1,2-a]pyridin-2-yl)methyl)amino)-6-hydroxypyridin-2-yl)cyclopropane-1-carboxamide (I-611);
(1S,2S)-2-(3-chlorophenyl)-N-(5-(((6-cyclopropyl-8-(4-methylpiperazin-1-yl)imidazo[1,2-a]pyridin-2-yl)methyl)amino)pyridazin-3-yl)cyclopropane-1-carboxamide (I-612);
(1S,2S)-2-(3-chlorophenyl)-N-(2-(((6-cyclopropyl-8-(4-methylpiperazin-1-yl)imidazo[1,2-a]pyridin-2-yl)methyl)amino)pyrimidin-4-yl)cyclopropane-1-carboxamide (I-613);
rac-(1S*,2S*)-2-(3-chloropyridinyl)-N-(5-(((6-cyclopropylimidazo[1,2-a]pyridin-2-yl)methyl)amino)-1,2,4-thiadiazol-3-yl)cyclopropane-1-carboxamide (I-614);
(1S,2S)-2-(3-chlorophenyl)-N-(5-(((6-cyclopropylimidazo[1,2-a]pyridin-2-yl)methyl)amino)-1,2,4-oxadiazol-3-yl)cyclopropane-1-carboxamide (I-615);
(1S,2S)-2-(3-chlorophenyl)-N-(6-((2R,4S)-2-(6-cyclopropylimidazo[1,2-a]pyridin-2-yl)-4-methoxypyrrolidin-1-yl)pyrimidin-4-yl)cyclopropane-1-carboxamide (I-616);
rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclopropane-1-carboxamide (I-617);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-(trifluoromethyl)pyridin-2-yl)-2-(4-methylpyrimidin-2-yl)cyclopropane-1-carboxamide (I-618);
rac-(1S*,2S*)—N-(5-(((6-cyclopropylimidazo[1,2-a]pyridin-2-yl)methyl)amino)-2-(methylsulfonyl)phenyl)-2-(4-methylpyrimidin-2-yl)cyclopropane-1-carboxamide (I-619);
rac-(1S*,2S*)—N-(6-((6-cyclopropyl-8-(3-methyl-2,4-dioxoimidazolidin-1-yl)imidazo[1,2-a]pyridin-2-yl)methyl)pyrimidin-4-yl)-2-(4-methylpyrimidin-2-yl)cyclopropane-1-carboxamide (I-620);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide (I-621);
rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-622);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-623);
rac-(1S*,2S*)—N-(7-(((6-cyclopropyl-8-(3-methyl-2,4-dioxoimidazolidin-1-yl)imidazo[1,2-a]pyridin-2-yl)methyl)amino)-[1,2,4]triazolo[1,5-a]pyrimidin-5-yl)-2-(4-methylpyrimidin-2-yl)cyclopropane-1-carboxamide (I-624);
N-(6-(2-(6-cyclopropyl-8-(3-methyl-2,4-dioxoimidazolidin-1-yl)imidazo[1,2-a]pyridin-2-yl)ethyl)pyrimidin-4-yl)-2-(4-methylpyrimidin-2-yl)cyclopropane-1-carboxamide (I-625);
rac-(1S*,2S*)—N-(3-((6-cyclopropyl-8-(3-methyl-2,4-dioxoimidazolidin-1-yl)imidazo[1,2-a]pyridin-2-yl)methyl)-2-oxo-2,3-dihydrooxazolo[4,5-d]pyrimidin-5-yl)-2-(4-methylpyrimidin-2-yl)cyclopropane-1-carboxamide (I-626);
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(hydroxymethyl)pyrimidin-2-yl)-2-(4-methylpyrimidin-2-yl)cyclopropane-1-carboxamide (I-627);
rac-(1S*,2S*)—N-(4-((6-cyclopropyl-8-(3-methyl-2,4-dioxoimidazolidin-1-yl)imidazo[1,2-a]pyridin-2-yl)methoxy)-5-(trifluoromethyl)pyrimidin-2-yl)-2-(4-methylpyrimidin-2-yl)cyclopropane-1-carboxamide (I-628);
(1S,2S)—N-(2-((6-cyclopropyl-8-(3-methyl-2,4-dioxoimidazolidin-1-yl)imidazo[1,2-a]pyridin-2-yl)methyl)oxazolo[5,4-c]pyridin-4-yl)-2-(4-methylpyrimidin-2-yl)cyclopropane-1-carboxamide (I-629);
(1S,2S)—N-(3-((6-cyclopropyl-8-(3-methyl-2,4-dioxoimidazolidin-1-yl)imidazo[1,2-a]pyridin-2-yl)methyl)-[1,2,4]triazolo[4,3-a]pyrazin-6-yl)-2-(4-methylpyrimidin-2-yl)cyclopropane-1-carboxamide (I-630);
(1S,2S)-2-(4-chloropyrimidin-2-yl)-N-(8-((6-cyclopropyl-8-(3-methyl-2,4-dioxoimidazolidin-1-yl)imidazo[1,2-a]pyridin-2-yl)methyl)-7,8-dihydro-6H-pyrimido[5,4-b][1,4]oxazin-2-yl)cyclopropane-1-carboxamide (I-631);
rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide (I-632);
rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide, first eluting isomer (I-633);
rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide, second eluting isomer (I-634);
(1R,2R)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide;
(1S,2S)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide;
(1S,2S)—N-(1-((6-cyclopropyl-8-(3-methyl-2,4-dioxoimidazolidin-1-yl)imidazo[1,2-a]pyridin-2-yl)methyl)-1H-[1,2,3]triazolo[4,5-c]pyridin-6-yl)-2-(4-methylpyrimidin-2-yl)cyclopropane-1-carboxamide (I-635);
(1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide (I-636);
(1S,2S)—N-(6-cyclopropyl-4-(((6-cyclopropyl-8-(3-(2-hydroxyethyl)-2,4-dioxoimidazolidin-1-yl)imidazo[1,2-a]pyridin-2-yl)methyl)(methyl)amino)pyridin-2-yl)-2-(4-methylpyrimidin-2-yl)cyclopropane-1-carboxamide (I-637);
(1S,2S)—N-(6-(((6-cyclopropyl-8-(3-methyl-2,4-dioxoimidazolidin-1-yl)imidazo[1,2-a]pyridin-2-yl)methyl)thio)-2-methylpyrimidin-4-yl)-2-(4-methylpyrimidin-2-yl)cyclopropane-1-carboxamide (I-638);
(1S,2S)—N-(2-(((6-cyclopropyl-8-(3-methyl-2,4-dioxoimidazolidin-1-yl)imidazo[1,2-a]pyridin-2-yl)methyl)amino)-6-methylpyridin-4-yl)-2-(4-methylpyrimidin-2-yl)cyclopropane-1-carboxamide (I-639);
(1S,2S)—N-(5-(((6-cyclopropyl-8-(3-methyl-2,4-dioxoimidazolidin-1-yl)imidazo[1,2-a]pyridin-2-yl)methyl)amino)-6-(trifluoromethyl)pyridazin-3-yl)-2-(4-methylpyrimidin-2-yl)cyclopropane-1-carboxamide (I-640);
(1S,2S)-2-(3-chlorophenyl)-N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)cyclopropane-1-carboxamide (I-641);
(1S,2S)—N-(4-(((6-cyclopropyl-8-(3-methyl-2,4-dioxoimidazolidin-1-yl)imidazo-[1,2-a]pyridin-2-yl)methyl)amino)-6-methylpyridin-2-yl)-2-(4-methylpyrimidin-2-yl)cyclopropane-1-carboxamide (I-642);
(1S,2S)—N-(3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (I-643);
(1S,2S)—N-(3-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (I-644);
(1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide (I-645);
(1S,2S)—N-(3-(((6-cyclopropyl-8-(3-methyl-2,4-dioxoimidazolidin-1-yl)imidazo[1,2-a]pyridin-2-yl)methyl)sulfonyl)phenyl)-2-(4-methylpyrimidin-2-yl)cyclopropane-1-carboxamide (I-646);
(1S,2S)-2-(3-chlorophenyl)-N-(6-(((6-cyclopropyl-8-(2-oxopiperazin-1-yl)imidazo[1,2-a]pyridin-2-yl)methyl)amino)pyrimidin-4-yl)cyclopropane-1-carboxamide (I-647);
(1S,2S)—N-(6-((6-cyclopropyl-8-(3-methyl-2,4-dioxoimidazolidin-1-yl)imidazo[1,2-a]pyridin-2-yl)methoxy)pyridazin-4-yl)-2-(4-methylpyrimidin-2-yl)cyclopropane-1-carboxamide (I-648);
(1S,2S)—N-(2-((6-cyclopropyl-8-(3-methyl-2,4-dioxoimidazolidin-1-yl)imidazo[1,2-a]pyridin-2-yl)methyl)pyrazolo[1,5-a]pyrazin-4-yl)-2-(4-methylpyrimidin-2-yl)cyclopropane-1-carboxamide (I-649);
(1S,2S)—N-(6-((1-(6-cyclopropyl-8-(3-methyl-2,4-dioxoimidazolidin-1-yl)imidazo [1,2-a]pyridin-2-yl)-2-hydroxyethyl)amino)-2-methylpyrimidin-4-yl)-2-(4-methylpyrimidin-2-yl)cyclopropane-1-carboxamide (I-650);
(1S,2S)—N-(5-(((6-cyclopropyl-8-(3-methyl-2,4-dioxoimidazolidin-1-yl)imidazo[1,2-a]pyridin-2-yl)methyl)amino)-1,2,4-triazin-3-yl)-2-(4-methylpyrimidin-2-yl)cyclopropane-1-carboxamide (I-651);
(1S,2S)—N-(2-((6-cyclopropyl-8-(3-methyl-2,4-dioxoimidazolidin-1-yl)imidazo[1,2-a]pyridin-2-yl)methoxy)-6-methylpyrimidin-4-yl)-2-(4-methylpyrimidin-2-yl)cyclopropane-1-carboxamide (I-652);
(1S,2S)—N-(4-(2-(6-cyclopropyl-8-(3-methyl-2,4-dioxoimidazolidin-1-yl)imidazo[1,2-a]pyridin-2-yl)ethyl)pyridin-2-yl)-2-(4-methylpyrimidin-2-yl)cyclopropane-1-carboxamide (I-653);
ethyl 4-((6-cyclopropyl-8-(3-methyl-2,4-dioxoimidazolidin-1-yl)imidazo[1,2-a]pyridin-2-yl)methoxy)-2-((1S,2S)-2-(4-methylpyrimidin-2-yl)cyclopropane-1-carboxamido)pyrimidine-5-carboxylate (I-654);
(1S,2S)—N-(3-cyano-5-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (I-655);
(1S,2S)—N-(3-(((6-cyclopropyl-8-(3-methyl-2,4-dioxoimidazolidin-1-yl)imidazo[1,2-a]pyridin-2-yl)methyl)amino)-4-fluorophenyl)-2-(4-methylpyrimidin-2-yl)cyclopropane-1-carboxamide (I-656);
(1S,2S)—N-(3-((6-cyclopropyl-8-(3-methyl-2,4-dioxoimidazolidin-1-yl)imidazo[1,2-a]pyridin-2-yl)methoxy)-4-fluorophenyl)-2-(4-methylpyrimidin-2-yl)cyclopropane-1-carboxamide (I-657);
(1S,2S)—N-(4-chloro-3-(((6-cyclopropyl-8-(3-methyl-2,4-dioxoimidazolidin-1-yl)imidazo[1,2-a]pyridin-2-yl)methyl)amino)-5-fluorophenyl)-2-(4-methylpyrimidin-2-yl)cyclopropane-1-carboxamide (I-658);
(1S,2S)—N-(5-(((6-cyclopropyl-8-(3-methyl-2,4-dioxoimidazolidin-1-yl)imidazo[1,2-a]pyridin-2-yl)methyl)amino)-2,4-difluorophenyl)-2-(4-methylpyrimidin-2-yl)cyclopropane-1-carboxamide (I-659);
(1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide (I-660);
(1S,2S)—N-(6-((6-cyclopropyl-8-(3-methyl-2,4-dioxoimidazolidin-1-yl)imidazo[1,2-a]pyridin-2-yl)methoxy)-4-hydroxypyridin-2-yl)-2-(4-methylpyrimidin-2-yl)cyclopropane-1-carboxamide (I-661);
(1S,2S)—N-(6-((6-cyclopropyl-8-(3-methyl-2,4-dioxoimidazolidin-1-yl)imidazo[1,2-a]pyridin-2-yl)methoxy)-2-(1-hydroxyethyl)pyrimidin-4-yl)-2-(4-methylpyrimidin-2-yl)cyclopropane-1-carboxamide (I-662);
(1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide (I-663);
(1S,2S)—N-(4-((6-cyclopropyl-8-(3-methyl-2,4-dioxoimidazolidin-1-yl)imidazo[1,2-a]pyridin-2-yl)methoxy)-5-(hydroxymethyl)pyrimidin-2-yl)-2-(4-methylpyrimidin-2-yl)cyclopropane-1-carboxamide (I-664);
(1S,2S)—N-(5-(((R)-1-(6-cyclopropyl-8-(3-methyl-2,4-dioxoimidazolidin-1-yl)imidazo[1,2-a]pyridin-2-yl)ethyl)amino)-1,2,4-triazin-3-yl)-2-(4-methylpyrimidin-2-yl)cyclopropane-1-carboxamide (I-665);
N-(5-((6-cyclopropyl-8-(3-methyl-2,4-dioxoimidazolidin-1-yl)imidazo[1,2-a]pyridin-2-yl)methoxy)-6-(trifluoromethyl)pyridazin-3-yl)-2-(4-methylpyrimidin-2-yl)cyclopropane-1-carboxamide (I-666);
(1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (I-667);
(1S,2S)—N-(5-(((6-cyclopropyl-8-(3-methyl-2,4-dioxoimidazolidin-1-yl)imidazo[1,2-a]pyridin-2-yl)methyl)(methyl)amino)-1,2,4-triazin-3-yl)-2-(4-methylpyrimidin-2-yl)cyclopropane-1-carboxamide (I-668);
(1S,2S)—N-(4-cyano-3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (I-669);
(1S,2S)—N-(5-((6-cyclopropyl-8-(3-methyl-2,4-dioxoimidazolidin-1-yl)imidazo[1,2-a]pyridin-2-yl)methoxy)-6-(methylamino)pyridazin-3-yl)-2-(4-methylpyrimidin-2-yl)cyclopropane-1-carboxamide (I-670);
(1S,2S)—N-(5-((6-cyclopropyl-8-(3-methyl-2,4-dioxoimidazolidin-1-yl)imidazo[1,2-a]pyridin-2-yl)methoxy)-1,2,4-triazin-3-yl)-2-(4-methylpyrimidin-2-yl)cyclopropane-1-carboxamide (I-671);
(1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (I-672);
2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (I-673);
N-(4-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-674);
(1S,2S)—N-(6-((6-cyclopropyl-8-(3-methyl-2,4-dioxoimidazolidin-1-yl)imidazo[1,2-a]pyridin-2-yl)methoxy)-4-fluoropyridin-2-yl)-2-(4-methylpyrimidin-2-yl)cyclopropane-1-carboxamide (I-675);
(1S,2S)—N-(5-((6-(1-fluorocyclopropyl)-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (I-676);
(1S,2S)—N-(5-((6-cyclopropyl-8-(3-methyl-2,4-dioxoimidazolidin-1-yl)imidazo[1,2-a]pyridin-2-yl)methoxy)-6-(hydroxymethyl)pyridazin-3-yl)-2-(4-methylpyrimidin-2-yl)cyclopropane-1-carboxamide (I-677);
(1S,2S)—N-(6-(((R)-1-(6-(fluoromethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-678); and
(1S,2S)—N-(6-(((R)-1-(6-isopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-679);

and pharmaceutically acceptable salts thereof.

Compounds explicitly disclosed herein may be claimed as an individual compound, including where there is no reference to stereochemistry.

Processes for preparing compounds of the disclosure are described herein below.

### A. Pharmaceutical Compositions

In another aspect, the present invention provides pharmaceutical compositions comprising a compound of the present disclosure, including Formulae (I)-(VI-c) or a compound of Formulae (I)-(VI-c) and examples in combination with a pharmaceutically acceptable excipient (e.g., carrier).

The pharmaceutical compositions include optical isomers, diastereomers, or pharmaceutically acceptable salts of the inhibitors disclosed herein. A compound of Formulae (I)-(VI-c) included in the pharmaceutical composition may be covalently attached to a carrier moiety, as described above. Alternatively, a compound of Formulae (I)-(VI-c) included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

In some embodiments, a compound as described herein can be incorporated into a pharmaceutical composition for administration by methods known to those skilled in the art and described herein for provided compounds.

### D. Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). In some embodiments compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### E. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat HAE, such compositions will contain an amount of active ingredient effective to achieve the desired result (e.g. inhibiting PKa and/or decreasing the amount of bradykinin in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to PKa inhibition); presence of other diseases or other health-related problems; kind of concurrent treatment; and complications from any disease or treatment regimen. Other therapeutic regimens or agents can be used in conjunction with the methods and compounds of the invention.

For any provided compound or test agent, the therapeutically effective amount can be initially determined from cell culture assays. Target concentrations will be those concentrations of active compound(s) that are capable of decreasing PKa enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring PKa inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound. In some embodiments, a reference compound is a PKa inhibitor known in the art. In some embodiments, a reference compound is a PKa inhibitor selected from those disclosed in PCT Publication Number WO 2019/178129.

In some embodiments a compound of the disclosure or a pharmaceutical composition comprising the same is provided as a unit dose.

### F. Methods of Treatment

The present disclosure provides compounds and pharmaceutical compositions comprising the same for use in medicine i.e. for use in treatment. The present disclosure further provides the use of any compounds described herein for inhibiting the activity of PKa, which would be beneficial to treatment of PKa-mediated diseases and conditions. Exemplary PKa-mediated disorders include edema, which refers to swelling in the whole body of a subject or a part thereof due to inflammation or injury when small blood vessels become leaky and releases fluid into nearby tissues. In some examples, the edema is HAE. In other examples, the edema occurs in eyes, e.g., diabetic macular edema (DME). The present disclosure provides methods of inhibiting the activity of PKa. In certain embodiments, the application provides a method of inhibiting the activity of PKa in vitro via contacting any of the compounds described herein with PKa molecules in a sample, such as a biological sample. In certain embodiments, the application provides a method of inhibiting the activity of PKa in vivo via delivering an effective amount of any of the compounds described herein to a subject in need of the treatment through a suitable route.

In certain embodiments, the methods comprise administering to a subject in need thereof (e.g., a subject such as a human patient, for example with edema) any of the compounds described herein or a pharmaceutically acceptable salt thereof. In certain embodiments, the methods comprise administering a compound of Formulae (I)-(VI-c), or a pharmaceutically acceptable salt or composition thereof, to a subject in need thereof. In some embodiments, the method comprises administering a pharmaceutical composition comprising a compound of Formulae (I)-(VI-c), or a pharmaceutically acceptable salt to a subject in need thereof.

In certain embodiments, the subject to be treated by any of the methods described herein is a human patient having, suspected of having, or at risk for edema, for example, HAE or diabetic macular edema (DIE). A subject having an edema can be identified by routine medical examination, e.g., laboratory tests. A subject suspected of having an edema might show one or more symptoms of the disease/disorder. A subject at risk for edema can be a subject having one or more of the risk factors associated with the disease, for example, deficiency in C1-INH as for HAE.

In certain embodiments, provided herein are methods of alleviating one or more symptoms of HAE in a human patient who is suffering from an HAE attack. Such a patient can be identified by routine medical procedures. An effective amount of one or more of the provided compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In other embodiments, provided herein are methods or reducing the risk of HAE attack in a human HAE patient who is in quiescent stage. Such a patient can be identified based on various factors, including history of HAE attack. An effective amount of one or more of the compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In some embodiments, provided herein is prophylactic treatment of HAE in human patients having risk to HAE attacks with one or more of the compounds described herein. In some embodiments, patients suitable for prophylactic treatment of HAE are human subjects suffering from HAE (e.g., having history of HAE attacks). In some embodiments, patients suitable for such prophylactic treatment are human subjects where a physician determines a history of HAE attacks warrants a prophylactic approach (e.g., human subjects experiencing more than a particular average number of attacks over a time period, including by way of nonlimiting example, one, two, or more attacks per month). Alternatively, patients suitable for the prophylactic treatment may be human subjects having no HAE attack history but bearing one or more risk factors for HAE (e.g., family history, genetic defects in C1-INH gene, etc.) Such prophylactic treatment may involve the compounds described herein as the sole active agent, or involve additional anti-HAE agents, such as those described herein.

In certain embodiments, provided herein are methods for preventing or reducing edema in an eye of a subject (e.g., a human patient). In some examples, the human patient is a diabetic having, suspected of having, or at risk for diabetic macular edema (DIE). DIE is the proliferative form of diabetic retinopathy characterized by swelling of the retinal layers, neovascularization, vascular leak, and retinal thickening in diabetes mellitus due to leaking of fluid from blood vessels within the macula. To practice this method, an effective amount of one or more of the compounds described herein, or pharmaceutically acceptable salts thereof, may be delivered into the eye of the subject where treatment is needed. For example, the compound may be delivered topically, by intraocular injection, or intravitreal injection. A subject may be treated with the compound as described herein, either as the sole active agent, or in combination with another treatment for DME. Non-limiting examples of treatment for DME include laser photocoagulation, steroids, VEGF pathway targeting agents (e.g., Lucentis® (ranibizumab) or Eylea® (aflibercept)), and/or anti-PDGF agents.

In certain embodiments, the methods disclosed herein comprise administering to the subject an effective amount of a compound of Formulae (I)-(VI-c), or a pharmaceutically acceptable salt or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formulae (I)-(VI-c), or at different times than the compound of Formulae (I)-(VI-c). For example, the compound of Formulae (I)-(VI-c) and any additional pharmaceutical agent(s) may be on the same dosing schedule or different dosing schedules. All or some doses of the compound of Formulae (I)-(VI-c) may be administered before all or some doses of an additional pharmaceutical agent, after all or some does an additional pharmaceutical agent, within a dosing schedule of an additional pharmaceutical agent, or a combination thereof. The timing of administration of the compound of Formulae (I)-(VI-c) and additional pharmaceutical agents may be different for different additional pharmaceutical agents.

Also provided is use of a compound of the present disclosure for the manufacture of a medicament for a condition/disease disclosed herein.

In certain embodiments, the additional pharmaceutical agent comprises an agent useful in the treatment of an edema, such as HAE or DME. Examples of such agents are provided herein.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of examples.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

The background section of this specification contains relevant technical information and may be used as basis for amendment. Subject headings herein are employed to divide the document into sections and are not intended to be used to construe the meaning of the disclosure provided herein.

The present specification claims priority from U.S. Provisional Application No. 63/162,468 (filed Mar. 17, 2021) incorporated herein by reference. This application may be used as basis for corrections to the present specification, especially in respect of chemical structures disclosed therein.

### IV. EXAMPLES

In certain embodiments, the Examples describe compounds comprising one or more stereocenters, where a particular stereocenter is designated “S*” or “R*.” In both cases, the depiction of the “*” generally indicates that the exact configuration is unknown (e.g., for a compound with a single stereocenter, the depiction R*- or S*- indicates that either the R- or S-isomer was isolated, but the configuration at the stereocenter of the particular isomer isolated was not determined).

It will be appreciated that compounds described within the Examples may comprise more than one stereocenter. As described above, single stereochemical isomers as well as enantiomeric, diastereomeric, and geometric (or conformational) mixtures of the present compounds are within the scope of the invention. Within a particular compound name, where more than one “S*” or “R*” appear within a single pair of parentheses (e.g., “(1S*,2S*)”), it is understood that the S* and/or R* configurations are relative to each other. For example, a compound denoted “(1S*,2S*)-” or “(1R*,2R*)-” would be understood to refer specifically to either the “(1S,2S)-” or “(1R,2R)-” isomer, but not the “(1S,2R)-” or “(1R,2S)-” isomers. Furthermore, a compound denoted “rac-(1S*,2S*)-” or “rac-(1R*,2R*)-” would be understood to include a racemic mixture of the “(1S,2S)-” and “(1R,2R)-” isomers. Similarly, a compound denoted “(1S*,2R*)-” or “(1R*,2S*)-” would be understood to refer specifically to either the “(1R,2S)-” or “(1S,2R)-” isomer, but not the “(1S,2S)-” or “(1R,2R)-” isomers. In addition, a compound denoted “rac-(1R*,2S*)-” or “rac-(1S*,2R*)-” would be understood to include a racemic mixture of the “(1R,2S)-” and “(1S,2R)-” isomers. In certain embodiments, the Examples include schemes that depict compounds with one or more stereocenters. In some embodiments, the symbol “&” followed by a number appears adjacent to a stereocenter. In such cases, it is understood to include a mixture of both configurations (e.g., R- and S-) at that position.

In some embodiments, the term “or” followed by a number appears adjacent to a stereocenter. In such cases, it is understood to denote either an “R-” or “S-” isomer, but the particular isomer was not determined.

In some embodiments, the numbering following the symbol “&” or term “or” refers to one stereocenter's relation to another stereocenter in that compound. For example, where two stereocenters in a compound are each denoted with the same number (e.g., two instances of “&1”), it is understood that the configurations are relative to each other (e.g., if the structure is drawn as (S,S) and both stereocenters are denoted “&1”, it is understood to include a mixture of the (S,S) and (R,R) isomers, but not the (S,R) or (R,S) isomers). However, where each stereocenter is denoted with a different number (e.g., one instance of “&1” and one instance of “&2”), it is understood that that the configurations may be independent to each other (e.g., if the structure is drawn (S,S) and one stereocenter is denoted “&1” and one is denoted “&2,” it is understood to include a mixture of the (S,S), (S,R), (R,S), and (R,R) isomers).

In one embodiment, Examples 449 to 453 can be prepared as follows:

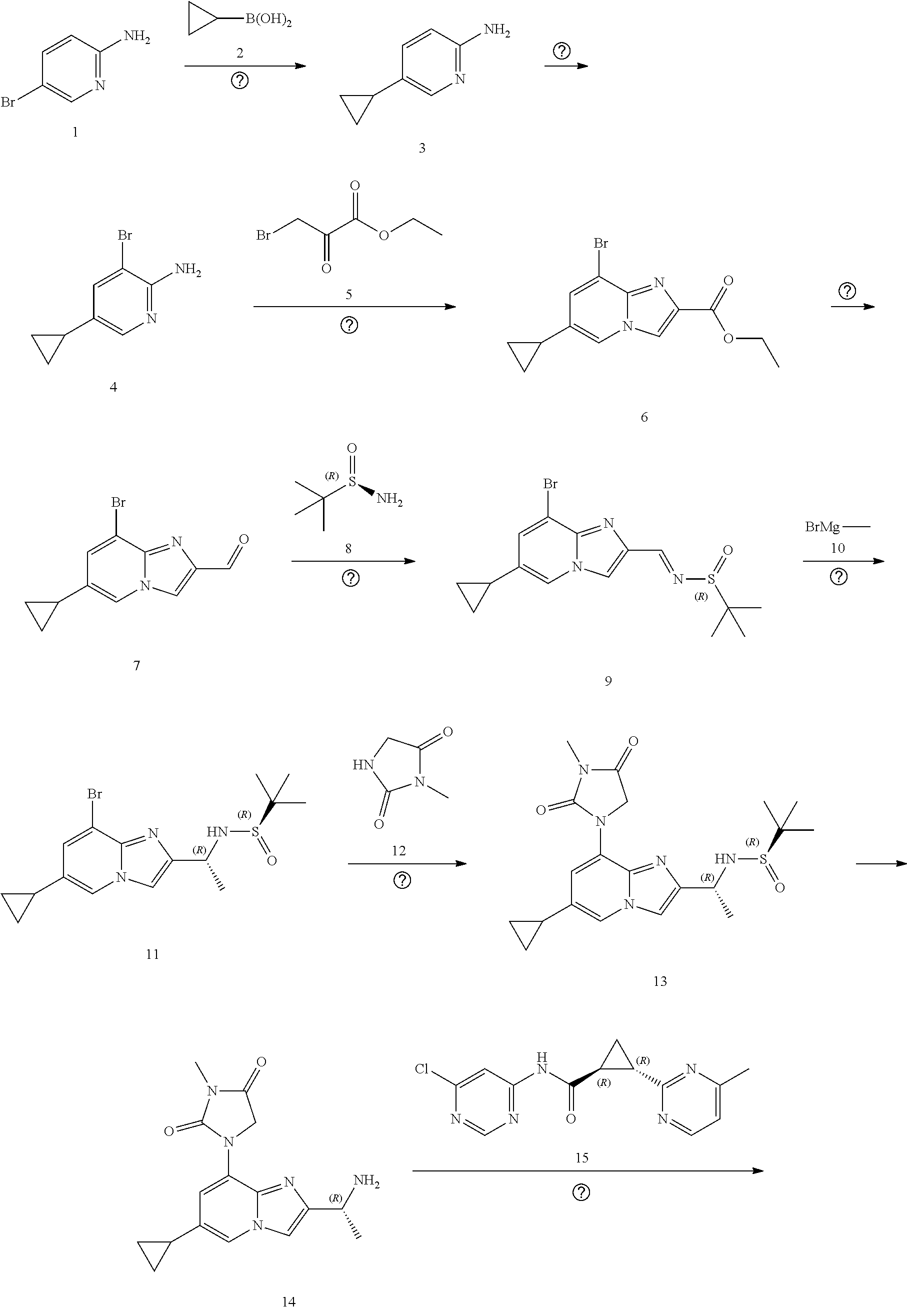

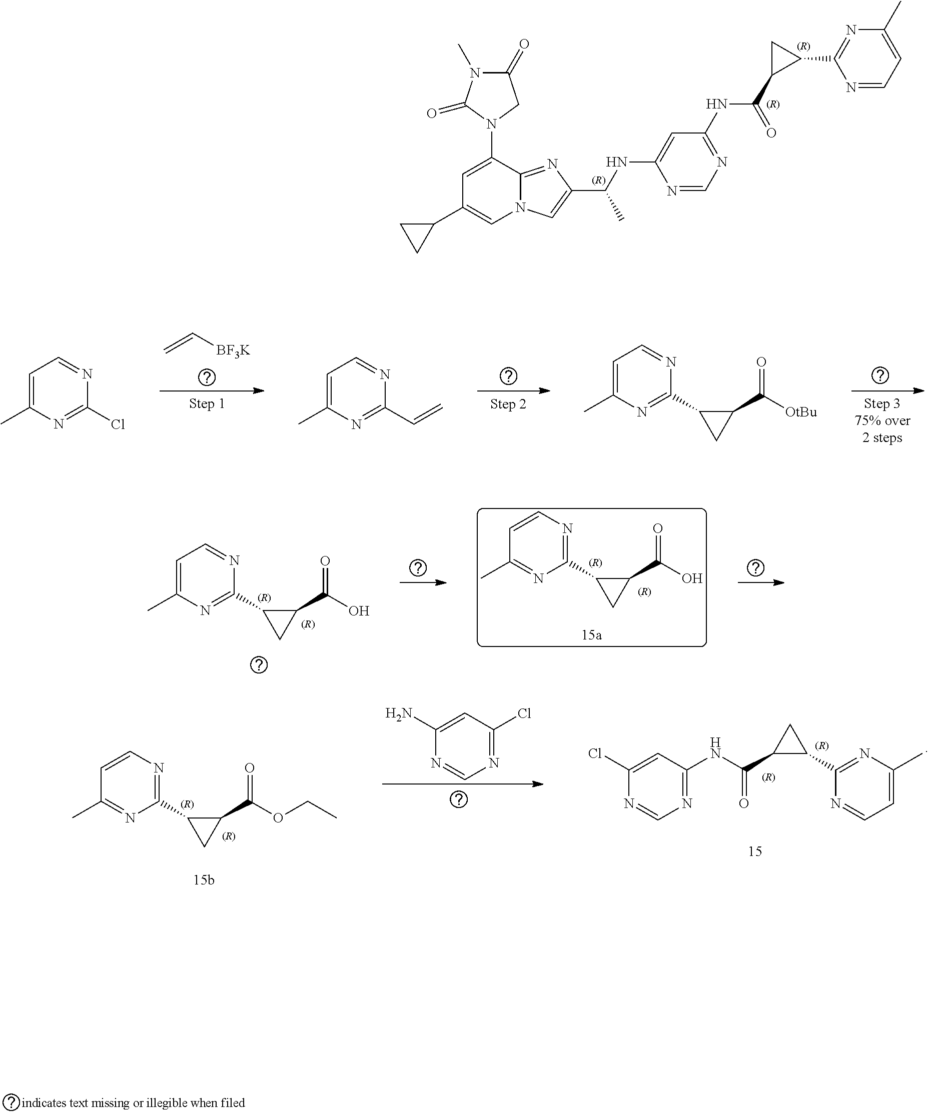

### Synthesis of Intermediates

### Synthesis of 4,6-dichloro-2-(methoxymethyl)pyrimidine

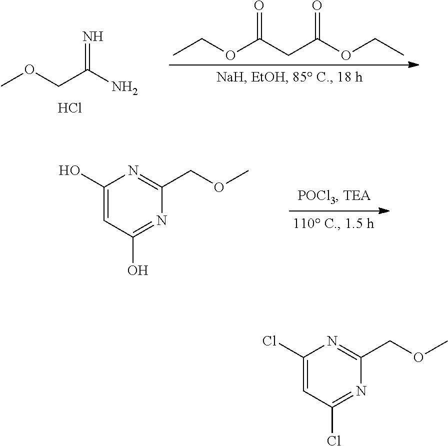

### Synthesis of 2-(methoxymethyl)pyrimidine-4,6-diol

To a mixture of 2-methoxyacetimidamide hydrochloride (5 g, 40.32 mmol) and diethyl malonate (9 g, 56.82 mmol) in EtOH (50 mL) was added NaH (5.6 g, 60% suspension in paraffin oil, 142.05 mmol) in small portions at room temperature and the resulting mixture was heated to 85° C. for 18 h. The reaction was cooled, the pH was adjusted to ˜3 with 4 N HCl solution, and the mixture was extracted with CHCl₃/IPA (3/1; 100 mL×8). The combined organic layers were washed with brine (50 mL), dried over Na₂SO₄ and concentrated in vacuo to afford 2-(methoxymethyl)pyrimidine-4,6-diol (4.8 g, 76%) as an off-white solid, which was used without further purification.

ESI-MS [M+H]+: 157.1.

### Synthesis of 4,6-dichloro-2-(methoxymethyl)pyrimidine

A mixture of 2-(methoxymethyl)pyrimidine-4,6-diol (2.5 g, 16 mmol) and TEA (2.4 g, 24 mmol) in POCl₃ (15.7 g, 103 mmol) was stirred at 110° C. for 1.5 h. The reaction was cooled to room temperature and concentrated. Ice-water (50 mL) was added and the mixture was extracted with EtOAc (50 mL×3). The combined organic layers were washed with brine (50 mL), dried over Na₂SO₄ and concentrated in vacuo. The residue was purified by silica gel chromatography (EtOAc/PE from 0 to 10%) to 4,6-dichloro-2-(methoxymethyl)pyrimidine (950 mg, 30.6% yield) as a yellow solid.

### Synthesis of 2-(2-((tert-butyldimethylsilyl)oxy)ethoxy)-4,6-dichloropyrimidine

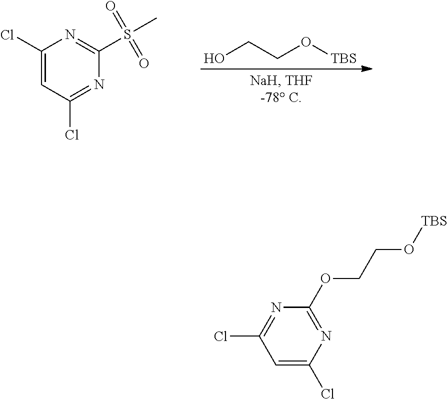

To a solution of 2-((tert-butyldimethylsilyl)oxy)ethan-1-ol (1.76 g, 10 mmol) in THF (30 mL) was added NaH (480 mg, 60% suspension in paraffin oil, 12 mmol) at 0° C. slowly. The resulting suspension was stirred at 0° C. for 10 min followed by addition of a solution of 4,6-dichloro-2-(methylsulfonyl) pyrimidine (2.27 g, 10 mmol) in THF (10 mL). After stirring at room temperature for 3 h, the reaction was quenched with saturated aqueous NH₄Cl (30 mL) and extracted with EtOAc (50 mL×3). The combined organic layers were washed with brine (50 mL), dried over Na₂SO₄, concentrated in vacuo and the crude product was purified by silica gel chromatography (eluent: PE/EtOAc=5/1) to give 2-(2-((tert-butyldimethylsilyl)oxy)ethoxy)-4,6-dichloropyrimidine (1.3 g, 40%) as a yellow solid. ESI-MS [M+H]+: 323.1.

### Synthesis of 4,6-dichloro-2-(difluoromethyl)pyrimidine

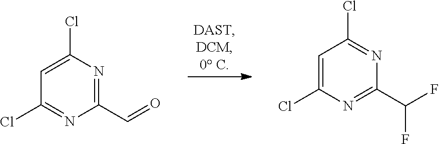 [SMILES: O=Cc1nc(Cl)cc(Cl)n1 ; FC(F)c1nc(Cl)cc(Cl)n1]

To a mixture of 4,6-dichloropyrimidine-2-carbaldehyde (150 mg, 0.86 mmol) in DCM (3 mL) was added DAST (694 mg, 4.3 mmol) at 0° C. under N₂. The reaction was stirred for 2 hours at room temperature under N₂. The reaction was quenched with H₂O (0.5 mL), then concentrated in vacuo and purified by preparative TLC (eluent: PE/EtOAc=6/1) to give 4,6-dichloro-2-(difluoromethyl)pyrimidine as a white solid (80 mg, 47%).

### Synthesis of 4,6-dichloro-2-(fluoromethyl)pyrimidine

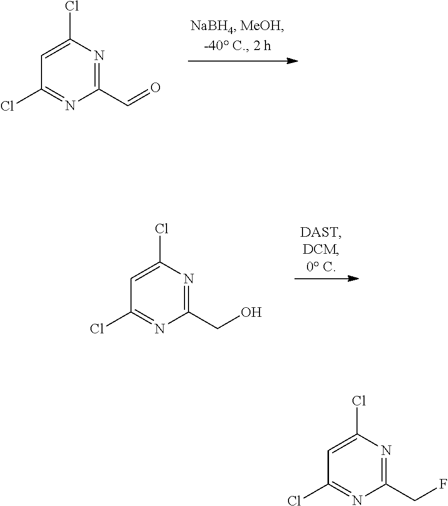 [SMILES: O=Cc1nc(Cl)cc(Cl)n1 ; OCc1nc(Cl)cc(Cl)n1 ; FCc1nc(Cl)cc(Cl)n1]

### Synthesis of (4,6-dichloropyrimidin-2-yl)methanol

To a mixture of 4,6-dichloropyrimidine-2-carbaldehyde (200 mg, 1.14 mmol) in MeOH (3 mL) was added NaBH₄ (86 mg, 2.28 mmol) at −40° C. under N₂. The reaction mixture was stirred at −40° C. for 1 h under N₂. The reaction was quenched with H₂O (3 mL) and extracted with EtOAc (10 mL×3). The organic layers were washed with brine (20 mL), dried over Na₂SO₄ then concentrated in vacuo to give (4,6-dichloropyrimidin-2-yl)methanol as a white solid (140 mg, crude), which was used directly in the next step. ESI-MS [M+H]⁺: 179.1

### Synthesis of 4,6-dichloro-2-(fluoromethyl)pyrimidine

A mixture of (4,6-dichloropyrimidin-2-yl)methanol (100 mg, 0.56 mmol) in DCM (5 mL) was added DAST (451 mg, 2.8 mmol) at 0° C. under N₂. The mixture was stirred for 2 h at RT under N₂. The mixture was quenched with H₂O (20 mL) and extracted with EtOAc (10 mL×3). The organic layers were concentrated in vacuo and purified by preparative TLC ((eluent: PE/EtOAc=6/1) to give the product 4,6-dichloro-2-(fluoromethyl)pyrimidine as a white solid (50 mg, 50%). ESI-MS [M+H]⁺: 181.2

### Synthesis of 2-bromo-6-(2-morpholinoethyl)pyridin-4-amine

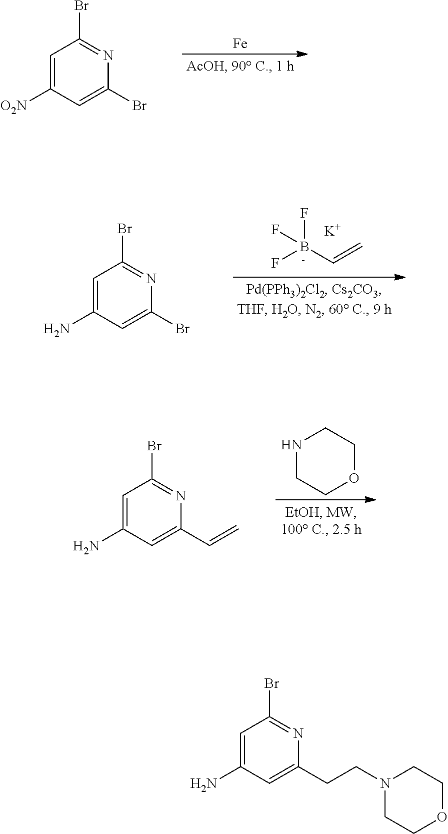

### Synthesis of 2,6-dibromopyridin-4-amine

To a mixture of 2,6-dibromo-4-nitropyridine (2.3 g, 8.2 mmol) in AcOH (15 ml) was added iron powder (2.3 mg, 41 mmol) at room temperature. The mixture was stirred at 90° C. for 1 h. The mixture was filtered and concentrated in vacuo to afford 2,6-dibromopyridin-4-amine (3.15 g, crude) as a yellow solid, which was used without further purification. ESI-MS [M+H]+: 281.1.

### Synthesis of 2-bromo-6-vinylpyridin-4-amine

To a mixture of 2,6-dibromopyridin-4-amine (3.0 g, 11.9 mmol), potassium trifluoro(vinyl)borate (1.6 g, 11.9 mmol) and Cs₂CO₃ (11.6 g, 35.7 mmol) in THF (27 ml) and water (3 mL) was added Pd(PPh₃)₂Cl₂ (417 mg, 0.60 mmol) at room temperature. The mixture was stirred at 60° C. for 9 h and cooled to room temperature. Water (50 mL) was added to the mixture and extracted with EtOAc (3×50 mL). The combined organic layers were washed with brine (60 mL), dried over anhydrous Na₂SO₄, filtered and concentrated in vacuo to give the crude, which was purified by silica gel chromatography (eluent: PE/EtOAc=3/1) to give 2-bromo-6-vinylpyridin-4-amine (584 mg, 25%) as a white solid. ESI-MS [M+H]+: 199.1.

### Synthesis of 2-bromo-6-(2-morpholinoethyl)pyridin-4-amine

To a solution of 2-bromo-6-vinylpyridin-4-amine (580 mg, 2.9 mmol) in EtOH (4 mL) was added morpholine (5.0 g, 58 mmol). The mixture was stirred at 100° C. in a microwave reactor for 2.5 h. The reaction was concentrated in vacuo. The residue was purified by silica gel chromatography (eluent: MeOH/DCM=0-10%) to afford 800 mg yellow solid, which was purified again by preparative TLC (eluent: DCM:MeOH=7:1) to afford 2-bromo-6-(2-morpholinoethyl)pyridin-4-amine (419 mg, 51%) as pale-yellow solid. ESI-MS [M+H]+: 286.1.

### Synthesis of 4-chloro-2-((6-cyclopropylimidazo[1,2-a]pyridin-2-yl)methyl)-2H-[1,2,3]triazolo[4,5-c]pyridine

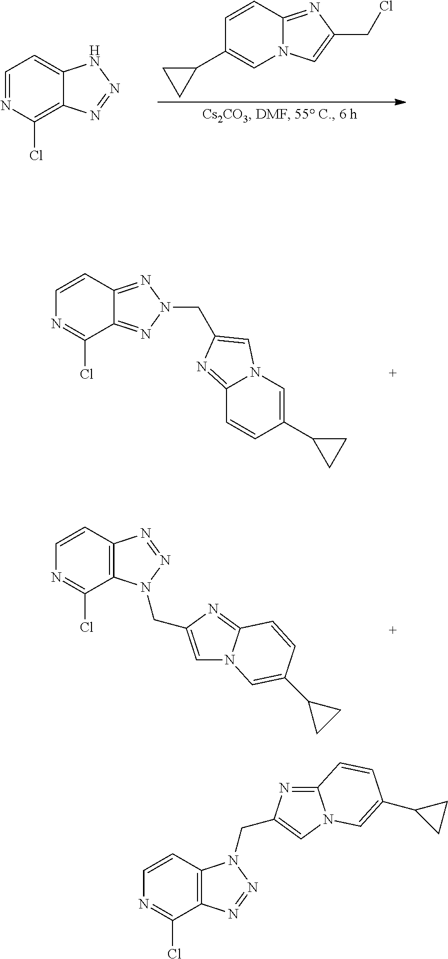

A mixture of 4-chloro-1H-[1,2,3]triazolo[4,5-c]pyridine (3.5 g, 22.6 mmol), 2-(chloromethyl)-6-cyclopropylimidazo[1,2-a]pyridine (5.6 g, 27.1 mmol) and Cs₂CO₃ (14.7 g, 45.2 mmol) in DMF (50 mL) was stirred at 55° C. for 6 h. After cooling to room temperature, the reaction was quenched with water (500 mL) and extracted with EtOAc (300 mL×3). The combined organic layers were washed with brine (300 mL), dried over Na₂SO₄, concentrated in vacuo to give the crude, which was purified by Prep-HPLC to give 4-chloro-2-((6-cyclopropylimidazo[1,2-a]pyridin-2-yl)methyl)-2H-[1,2,3]triazolo[4,5-c]pyridine (100 mg, 1.4%), 4-chloro-3-((6-cyclopropylimidazo[1,2-a]pyridin-2-yl)methyl)-3H-[1,2,3]triazolo[4,5-c]pyridine (250 mg, 3.4%) and 4-chloro-1-((6-cyclopropylimidazo[1,2-a]pyridin-2-yl)methyl)-1H-[1,2,3]triazolo[4,5-c]pyridine (1.5 g, 21%) as yellow solids. ESI-MS [M+H]+: 325.1

### Synthesis of methyl (E)-3-(5-chloropyridin-3-yl)acrylate

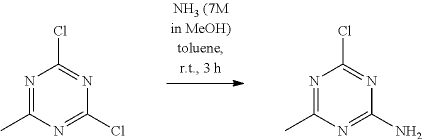

A mixture of 2,4-dichloro-6-methyl-1,3,5-triazine (700 mg, 4.29 mmol) in NH₃/MeOH (9 mL, 7M) and toluene (10 mL) was stirred at room temperature for 3 h. The mixture was concentrated in vacuo to give the crude, which was purified by silica gel chromatography (eluent: DCM/MeOH=10/1) to give 4-chloro-6-methyl-1,3,5-triazin-2-amine (300 mg, 49%) as a white solid. ESI-MS [M+H]+: 145.2.

### Synthesis of 4-chloro-6-methyl-2H-pyrazolo[4,3-c]pyridine

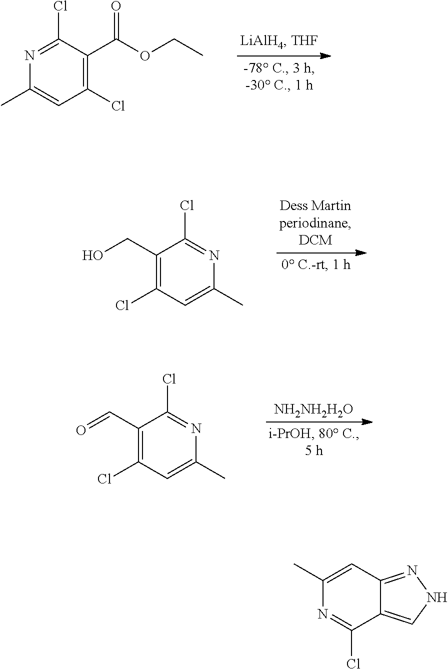

### Synthesis of (2,4-dichloro-6-methylpyridin-3-yl)methanol

To a solution of ethyl 2,4-dichloro-6-methylnicotinate (2.0 g, 8.54 mmol) in THF (30 mL) was added LiAlH₄ (42 mL, 42 mmol) at −78° C. The resulting mixture was stirred at −78° C. for 3 h and at −30° C. for another 1 h under N₂. After cooling back the mixture to −78° C., a solution of saturated aq·NH₄Cl (100 mL) solution was added and extracted with EtOAc (3×40 mL). The combined organic layers were washed with brine (50 mL), dried over Na₂SO₄ and concentrated in vacuo. The residue was purified by silica gel chromatography (EtOAc/PE from 0 to 10%) to give (2,4-dichloro-6-methylpyridin-3-yl)methanol (1.2 g, 73%) as a colorless oil. ESI-MS [M+H]⁺: 192.0.

### Synthesis of 2,4-dichloro-6-methylnicotinaldehyde

A mixture of (2,4-dichloro-6-methylpyridin-3-yl)methanol (1.2 g, 6.25 mmol) in DCM (30 mL) was added Dess Martin periodinane (3.18 g, 7.5 mmol) at 0° C. and warmed to room temperature for 1 h under N₂. The mixture quenched with saturated aq·Na₂S₂O₃ (50 mL) and extracted with DCM (3×50 mL). The combined organic layers were washed with brine (50 mL), dried over Na₂SO₄ and concentrated in vacuo. The residue was purified by preparative TLC (eluent: PE/EtOAc=10/1) to give 2,4-dichloro-6-methylnicotinaldehyde (1 g, 84%) as a white solid. ESI-MS [M+H]⁺: 190.0.

### Synthesis of 4-chloro-6-methyl-2H-pyrazolo[4,3-c]pyridine

To a mixture of 2,4-dichloro-6-methylnicotinaldehyde (1 g, 5.26 mmol) in i-PrOH (15 mL) was added hydrazine hydrate (1.5 mL, 45.6 mmol) and the mixture was stirred at 80° C. for 5 h under N₂. The reaction was quenched with water (30 mL) and the mixture was extracted with DCM (3×30 mL). The combined organic layers were washed with brine (50 mL), dried over Na₂SO₄ and concentrated in vacuo. The crude product was purified by preparative TLC (eluent: PE/EtOAc=10/1) to give 4-chloro-6-methyl-2H-pyrazolo[4,3-c]pyridine (370 mg, 42%) as a white oil. ESI-MS [M+H]⁺: 168.0.

### Synthesis of 2,4-dichloro-6-((4-methoxybenzyl)oxy)-1,3,5-triazine

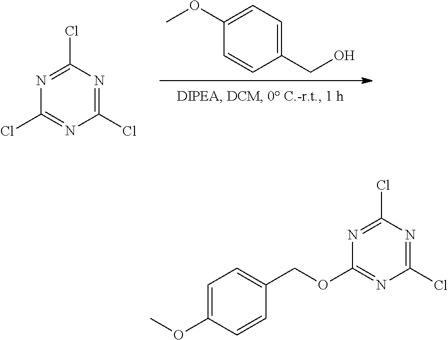

To a mixture of 2,4,6-trichloro-1,3,5-triazine (550 mg, 3.0 mmol) and (4-methoxyphenyl)methanol (414 mg, 3.0 mmol) in DCM (10 mL) was added DIPEA (774 mg, 6.0 mmol) at 0° C. and then stirred at room temperature under N₂ for 1 h. The resulting mixture was evaporated to afford crude 2,4-dichloro-6-((4-methoxybenzyl)oxy)-1,3,5-triazine (1.2 g, crude) as a yellow oil which was used directly in next step. ESI-MS [M+H]+: 286.0

### Synthesis of 4,6-dichloro-2-((4-methoxybenzyl)oxy)pyrimidine

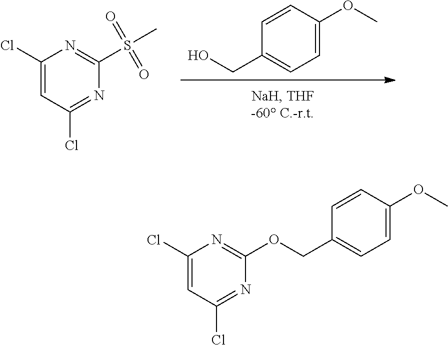

To a mixture of 4,6-dichloro-2-(methylsulfonyl)pyrimidine (800 mg, 3.54 mmol) in THF (15 mL) was added NaH (254 mg, 11 mmol) at −60° C. under N₂. After stirring the mixture at −60° C. for 1 h, a solution of (4-methoxyphenyl)methanol (586 mg, 4.24 mmol) in THF (5 mL) was added dropwise at −60° C. Then the mixture was warmed to room temperature and stirred for 1 h. The reaction was quenched with saturated aq·NH₄Cl (50 mL) and extracted with EtOAc (3×50 mL). The combined organic layers were washed with brine (30 mL), dried over anhydrous Na₂SO₄, and concentrated in vacuo to give the crude, which was purified by silica gel chromatography (eluent: MeOH/DCM=1/50) to give 4,6-dichloro-2-((4-methoxybenzyl)oxy)pyrimidine (400 mg, 40%) as a yellow solid. ESI-MS [M+H]+: 285.1

### Synthesis of 4,6-dichloro-2-(pyrrolidin-1-yl)pyrimidine

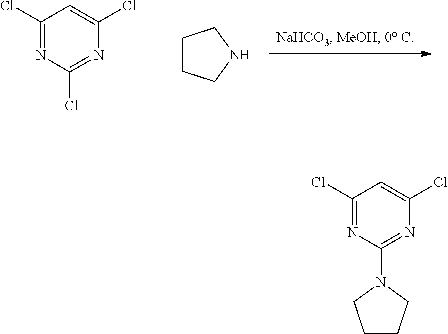 [SMILES: Clc1cc(Cl)nc(Cl)n1 ; C1CCNC1 ; Clc1cc(Cl)nc(N2CCCC2)n1]

A suspension of 2,4,6-trichloropyrimidine (1.0 g, 5.5 mmol) and NaHCO₃ (1.4 g, 16 mmol) in MeOH (7.0 mL) was stirred at 0° C. A solution of pyrrolidine (0.46 mL, 5.5 mmol) in MeOH (3 mL) was added dropwise and the mixture was stirred at 0° C. for 3 h. The reaction mixture was allowed to warm to room temperature, filtered, concentrated in vacuo. The residue was purified by column chromatography on silica gel, eluting with a gradient of 0-10% EtOAc in cyclohexane to give the title compound (420 mg, 35%).

¹H NMR (400 MHz, CDCl₃) δ 6.51 (s, 1H), 3.60-3.55 (m, 4H), 2.01-1.96 (m, 4H).

### Synthesis of ethyl 4,6-dichloropyrimidine-2-carboxylate

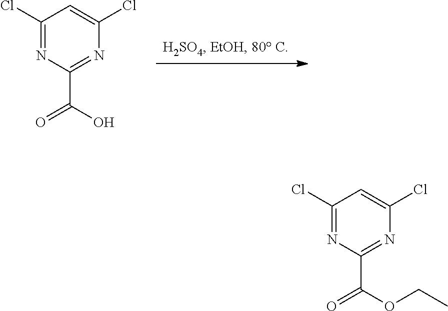

A solution of 4,6-dichloropyrimidine-2-carboxylic acid (300 mg, 1.6 mmol) and H₂SO₄ (8.3 μL, 0.16 mmol) in EtOH (8.0 mL) was stirred at 80° C. for 16 h. The reaction mixture was allowed to cool to room temperature and concentrated in vacuo. The residue was dissolved in DCM (15 mL) and washed with NaHCO₃ (sat·aq·, 10 mL), dried over MgSO₄ and concentrated in vacuo to give the title compound (270 mg, 78%) as a colourless oil.

ESI-MS (M+H): 221.0, ¹H NMR (400 MHz, CDCl₃) δ 7.58 (s, 1H), 4.54 (q, J=7.0 Hz, 2H), 1.46 (t, J=7.3 Hz, 3H).

### Synthesis of 2-(2-((tert-butyldimethylsilyl)oxy)ethyl)-4,6-dichloropyrimidine

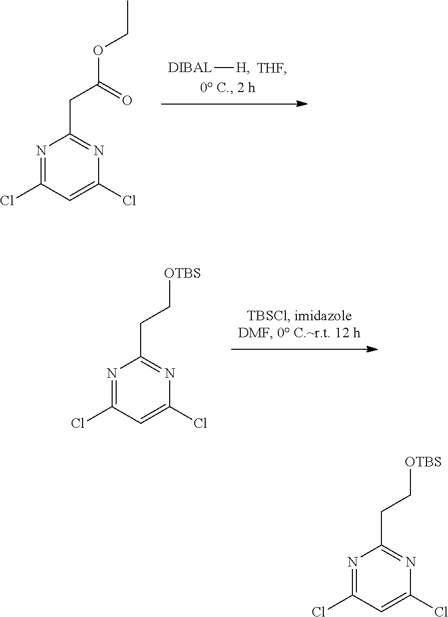

### Synthesis of 2-(4,6-dichloropyrimidin-2-yl)ethan-1-ol

To a solution of ethyl 2-(4,6-dichloropyrimidin-2-yl)acetate (117.5 mg, 0.5 mmol) in THF (5 mL) was added DIBALH (1.0 M in hexane, 1.0 mL, 1.0 mmol) at 0° C. After stirring the mixture at room temperature for 2 h, the reaction was quenched with Na₂SO₄·10 H₂O. The resulting solution was filtered and the filter cake was washed with DCM/MeOH (10/1, 50 mL). The filtrate was concentrated in vacuo to give the crude product 2-(4,6-dichloropyrimidin-2-yl)ethan-1-ol as a yellow solid (109 mg, crude), which was used without further purification.

ESI-MS [M+H]+: 193.1

### Synthesis of 2-(2-((tert-butyldimethylsilyl)oxy)ethyl)-4,6-dichloropyrimidine

To a solution of 2-(4,6-dichloropyrimidin-2-yl)ethan-1-ol (96 mg, 0.5 mmol) in DMF (5 mL) was added imidazole (51 mg, 0.75 mmol) and TBSCl (83 mg, 0.55 mmol) at 0° C. After stirring at room temperature for 3 h, the reaction was quenched with water (20 mL) then extracted with EtOAc (3×20 mL). The combined organic layers were washed with brine (30 mL), dried over Na₂SO₄ and concentrated in vacuo to give the crude product, which was purified by silica gel chromatography (PE:EA=10:1) to give the product 2-(2-((tert-butyldimethylsilyl)oxy)ethyl)-4,6-dichloropyrimidine as a colorless oil (75 mg, 49%, two steps).

ESI-MS [M+H]+: 307.1

### Synthesis of 2-(((tert-butyldimethylsilyl)oxy)methyl)-4,6-dichloropyrimidine

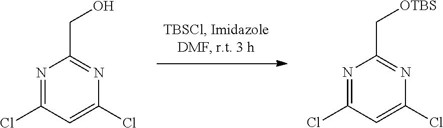 [SMILES: OCc1nc(Cl)cc(Cl)n1 ; CC(C)(C)[Si](C)(C)OCc1nc(Cl)cc(Cl)n1]

To a mixture of (4,6-dichloropyrimidin-2-yl)methanol (1.48 g crude) and imidazole (1.135 g, 16.686 mmol) in DCM (50 mL) was added TBSCl (1.89 g, 12.515 mmol) at 0° C. After stirring at room temperature for 3 h, the reaction mixture was quenched with water (40 mL) and extracted with DCM (3×50 mL). The combined organic layers were washed with brine (50 mL), dried over anhydrous Na₂SO₄, filtered and concentrated in vacuo. The residue was purified by silica gel column chromatography (eluent: PE/EtOAc=4/1) to give the product (1.72 g, 70%) as yellow oil.

ESI-MS [M+H]+: 293.1.

### Synthesis of 4,6-dichloro-N,N-dimethylpyrimidin-2-amine

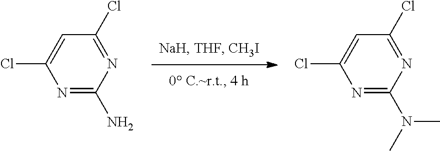

To a solution of 4,6-dichloropyrimidin-2-amine (1.0 g, 6.1 mmol) in THF (40 mL) was added NaH (1 g, 25 mmol, 60% dispersion in mineral oil) slowly at 0° C. The reaction mixture was stirred at 0° C. for 1 h, then CH₃I (1.7 g, 12 mmol) was added at 0° C. After stirring at room temperature for another 3 h, the reaction was quenched with water (40 mL) then extracted with EtOAc (3×50 mL). The combined organics were dried over Na₂SO₄ and concentrated in vacuo to give the crude, which was purified by silica gel column chromatography (eluent: PE/EtOAc=2/1) to afford 4,6-dichloro-N,N-dimethylpyrimidin-2-amine (640 mg, 55%) as a yellow solid. ESI-MS: [M+H]+, 192.0

### Synthesis of 2-(5-chloro-2-nitrophenyl)cyclopropane-1-carboxamide

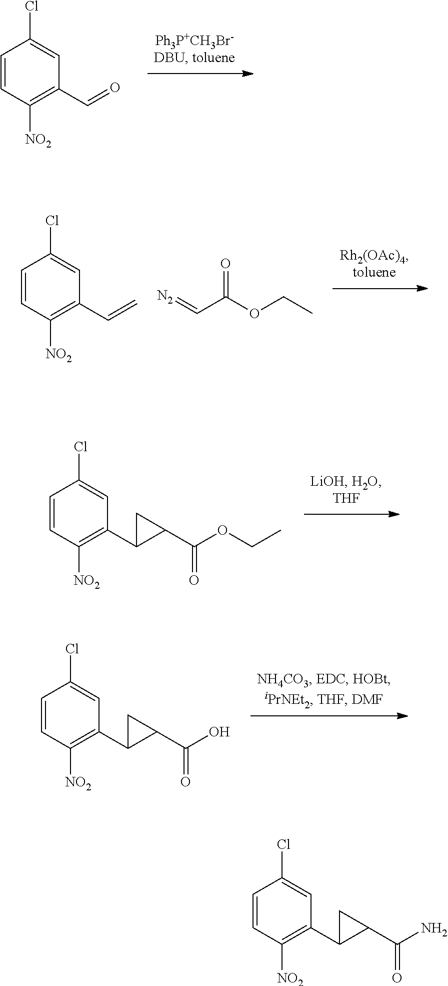

### Synthesis of 4-chloro-1-nitro-2-vinylbenzene

DBU (2.9 mL, 19.4 mmol) was added to a stirred solution of 5-chloro-2-nitrobenzaldehyde (3.0 g, 16.17 mmol) and methyltriphenylphosphonium bromide (6.93 g, 19.4 mmol) in dry toluene (50 mL). The mixture was stirred at room temperature for 16 h, then (NH₄)₂CO₃ (sat·aq·) was added. The mixture was extracted with EtOAc (×3) and the organic extracts were dried (hydrophobic frit) and concentrated in vacuo. The resulting oil was purified by dry flash silica chromatography eluting with a gradient of 0-50% EtOAc in cyclohexane to give the desired product (450 mg, 15%). ¹H NMR (400 MHz, CDCl₃) δ 7.95-7.90 (m, 1H), 7.61-7.57 (m, 1H), 7.38 (dd, J=2.1, 8.7 Hz, 1H), 7.23-7.12 (m, 1H), 5.76 (d, J=17.2 Hz, 1H), 5.54 (d, J=10.9 Hz, 1H).

### Synthesis of ethyl 2-(5-chloro-2-nitrophenyl)cyclopropane-1-carboxylate

A solution of ethyl diazoacetate (0.60 mL, 5.72 mmol) in toluene (5 mL) was added dropwise over 2 h to a solution of 4-chloro-1-nitro-2-vinylbenzene (350 mg, 1.91 mmol) and Rh₂(OAc)₄ (17 mg, 0.038 mmol) in toluene (5 mL) at 80° C. for 16 h. The reaction mixture was cooled to room temperature and concentrated in vacuo. The crude product was purified by dry flash silica chromatography eluting with a gradient of 0-20% EtOAc in cyclohexane to give the desired product as a 2:1 mixture of diastereoisomers (255 mg, 50%). ¹H NMR (400 MHz, CDCl₃) δ 7.92 (d, J=9.1 Hz, 1H), 7.36 (d, J=8.6 Hz, 1H), 7.23 (s, 1H), 4.30-4.19 (m, 2H), 3.02-2.95 (m, 1H), 1.90-1.83 (m, 1H), 1.72-1.67 (m, 1H), 1.35-1.28 (m, 4H).

### Synthesis of 2-(5-chloro-2-nitrophenyl)cyclopropane-1-carboxylic acid

LiOH (222 mg, 9.27 mmol) was added to a solution of ethyl 2-(5-chloro-2-nitrophenyl)cyclopropane-1-carboxylate (500 mg, 1.85 mmol) in THF/water (1:1; 10 mL) and the mixture was stirred at room temperature for 16 h. The reaction mixture was acidified to pH1 with HCl (1 M, aq·) and extracted with DCM. The organic extract was dried (hydrophobic frit) and concentrated in vacuo to give the desired product as a mixture of diastereoisomers (356 mg, 79%). ¹H NMR (400 MHz, DMSO) δ 8.06 (d, J=8.7 Hz, 1H), 7.67-7.60 (m, 2H), 7.52 (d, J=1.8 Hz, 1H), 2.80-2.73 (m, 1H), 1.98-1.91 (m, 1H), 1.65-1.60 (m, 1H), 1.51-1.46 (m, 1H). ESI-MS (M+H)+: 240

### Synthesis of 2-(5-chloro-2-nitrophenyl)cyclopropane-1-carboxamide

A solution of 2-(5-chloro-2-nitrophenyl)cyclopropane-1-carboxylic acid (150 mg, 0.62 mmol), EDC·HCl (116 mg, 0.745 mmol), HOBt (101 mg, 0.745 mmol), (NH₄)₂CO₃ (581 mg, 3.10 mmol) and DIPEA (0.65 mL, 3.72 mmol) in THF (10 mL) and DMF (10 mL) was stirred at room temperature for 16 h. Brine was added and the mixture was extracted with EtOAc. The organic extract was dried (hydrophobic frit) and concentrated in vacuo. The crude product was purified by dry flash silica chromatography eluting with a gradient of 0-100% EtOAc in cyclohexane to give the desired product as a mixture of diastereoisomers (56 mg, 37%). ¹H NMR (400 MHz, CDCl₃) δ 7.89 (d, J=8.6 Hz, 1H), 7.36 (dd, J=2.0, 8.8 Hz, 1H), 7.26 (s, 1H), 5.66 (d, J=54.9 Hz, 2H), 2.97-2.87 (m, 1H), 1.76-1.64 (m, 2H), 1.34-1.22 (m, 1H).

### Synthesis of 2-(6-cyano-2-fluoro-3-methoxyphenyl)cyclopropane-1-carboxamide

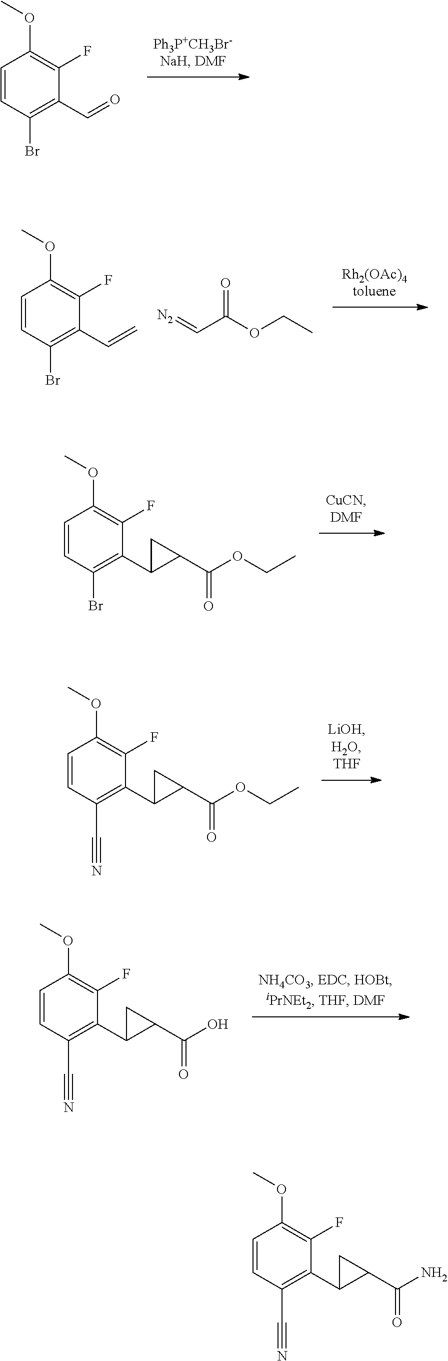

### Synthesis of 1-bromo-3-fluoro-4-methoxy-2-vinylbenzene

NaH (60% in mineral oil; 0.15 g, 6.44 mmol) was added to an ice-cooled solution of methyltriphenylphosphonium bromide (2.30 g, 6.44 mmol) in DMF (50 mL) at 0° C. The solution was allowed to warm to room temperature, stirred for 30 min, then cooled to 0° C. 6-Bromo-2-fluoro-3-methoxybenzaldehyde (5.78 g, 16.17 mmol) was added portion-wise. The mixture was stirred at room temperature for 16 h, then NH₄Cl (sat·aq·) was added. The mixture was extracted with EtOAc (×3) and the organic extracts were dried over MgSO₄ and concentrated in vacuo. The resulting oil was purified by flash silica chromatography eluting with a gradient of 0-15% EtOAc in isohexane to give the desired product as a clear oil (790 mg, 64%). ¹H NMR (400 MHz, CDCl₃) δ 7.29 (dd, J=2.1, 8.7 Hz, 1H), 6.79-6.65 (m, 2H), 5.92 (d, J=17.9 Hz, 1H), 5.65 (d, J=11.6 Hz, 1H), 3.88 (s, 3H).

### Synthesis of ethyl 2-(6-bromo-2-fluoro-3-methoxyphenyl)cyclopropane-1-carboxylate

A solution of ethyl diazoacetate (1.1 mL, 10.3 mmol) in toluene (3 mL) was added dropwise over 3 h to a solution of 1-bromo-3-fluoro-4-methoxy-2-vinylbenzene (790 mg, 3.43 mmol) and Rh₂(OAc)₄ (30 mg, 0.069 mmol) in toluene (7 mL) at 80° C. The reaction mixture was stirred at 80° C. for 16 h, cooled to room temperature and concentrated in vacuo. The crude product was partially purified by dry flash silica chromatography eluting with a gradient of 0-30% ethyl acetate in isohexane to give a ˜1:1 mixture of the desired product and the starting alkene (724 mg). The impure product was carried forward without further purification to the next step.

### Synthesis of ethyl 2-(6-cyano-2-fluoro-3-methoxyphenyl)cyclopropane-1-carboxylate

A mixture of ethyl 2-(6-bromo-2-fluoro-3-methoxyphenyl)cyclopropane-1-carboxylate (˜50% purity; 724 mg, 1.14 mmol), and CuCN (204 mg, 2.28 mmol) in DMF (11 mL) was heated at 150° C. for 16 h. The mixture was cooled to room temperature, then diluted with EtOAc and 1:4 NH₄OH (sat·aq·) in NH₄Cl (sat·aq·). The organic phase was separated and the aqueous phase further extracted with EtOAc (×2). The organic extracts were dried (MgSO₄) and concentrated in vacuo. The crude product was purified by dry flash silica chromatography eluting with 0-40% EtOAc in isohexane to give the desired product as a mixture of isomers (175 mg, 58%). This was used without further purification

### Synthesis of 2-(6-cyano-2-fluoro-3-methoxyphenyl)cyclopropane-1-carboxamide

Using a similar procedure to that for 2-(5-chloro-2-nitrophenyl)cyclopropane-1-carboxamide, 2-(6-cyano-2-fluoro-3-methoxyphenyl)cyclopropane-1-carboxamide (67 mg) was synthesised from ethyl 2-(6-cyano-2-fluoro-3-methoxyphenyl)cyclopropane-1-carboxylate (175 mg, 0.66 mmol) and was used without further purification in the next step.

### Synthesis of 2-(3-chlorophenyl)-2-fluorocyclopropane-1-carboxamide

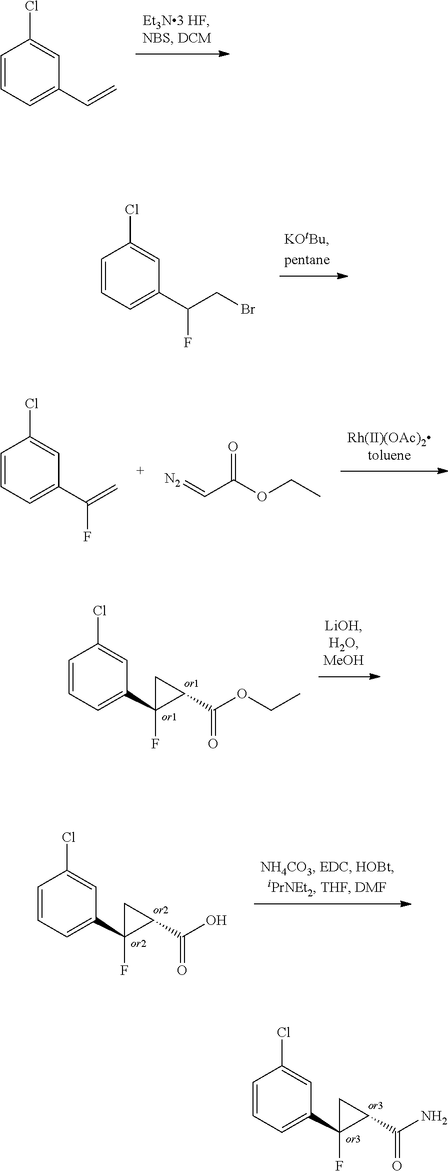

### Synthesis of rac-1-(2-bromo-1-fluoroethyl)-3-chlorobenzene

NEt₃·3HF (7.1 mL, 43.29 mmol) was added dropwise to an ice-cooled solution of 3-chlorostyrene (1.8 g, 14.43 mmol) and NBS (3.08 g, 17.32 mmol) in DCM (15 mL) at 0° C. and the mixture was stirred at room temperature for 16 h. Further NBS (1.50 g, 8.43 mmol) was added and the mixture was stirred at room temperature for 7 h. The solution was poured into NH₄OH (sat·aq·), then extracted with EtOAc (×5). The extracts were washed with HCl (1 M aq·), dried (MgSO₄) and concentrated in vacuo. The crude product was purified by dry flash silica chromatography eluting with pentane to give the desired product as a clear oil (1.61 g, 47%). ¹H NMR (400 MHz, CDCl₃) δ 7.38-7.33 (m, 3H), 7.26-7.21 (m, 1H), 5.68-5.52 (m, 1H), 3.71-3.55 (m, 2H).

### Synthesis of 1-chloro-3-(1-fluorovinyl)benzene

KO^{t}Bu (1.52 g, 13.56 mmol) was added portion-wise to an ice-cooled solution of rac-1-(2-bromo-1-fluoroethyl)-3-chlorobenzene (1.61 g, 6.78 mmol) in pentane (40 mL) at 0° C. and the mixture was stirred at 35° C. for 1.5 h. The suspension was poured into iced water, then the mixture was extracted with pentane (×2). The extract was a dried (MgSO₄) and concentrated in vacuo to give the desired product as a clear oil (0.93 g, 88%). ¹H NMR (400 MHz, CDCl₃) δ 7.53 (dd, J=1.9, 1.9 Hz, 1H), 7.45-7.41 (m, 1H), 7.34-7.30 (m, 2H), 5.06 (dd, J=3.6, 49.2 Hz, 1H), 4.90 (dd, J=3.8, 17.7 Hz, 1H).

### Synthesis of ethyl 2-(3-chlorophenyl)-2-fluorocyclopropane-1-carboxylate

The title compound was synthesised using a similar procedure to ethyl 2-(6-bromo-2-fluoro-3-methoxyphenyl)cyclopropane-1-carboxylate starting from 1-chloro-3-(1-fluorovinyl)benzene (200 mg, 1.28 mmol) to give the desired product as a mixture of diastereoisomers (279 mg, 90%).

¹H NMR (400 MHz, CDCl₃) δ 7.49-7.47 (m, 0.5H, Isomer A), 7.37-7.29 (m, 2.5H, Isomer A+B), 7.18-7.14 (m, 0.5H, Isomer B), 4.29-4.18 (m, 2H), 2.61-2.51 (m, 0.5H, Isomer A), 2.36-2.27 (m, 0.5H, Isomer B), 2.21-2.15 (m, 0.5H, Isomer A), 2.01-1.93 (m, 0.5H, Isomer B), 1.89-1.78 (m, 0.5H, Isomer B), 1.65-1.58 (m, 0.5H, Isomer A), 1.35-1.28 (m, 3H).

### Synthesis of 2-(3-chlorophenyl)-2-fluorocyclopropane-1-carboxamide

The title compound was synthesised using a similar procedure to 2-(6-cyano-2-fluoro-3-methoxyphenyl)cyclopropane-1-carboxamide starting from ethyl 2-(3-chlorophenyl)-2-fluorocyclopropane-1-carboxylate (279 mg, 1.15 mmol) to give the desired product as a mixture of diastereoisomers (yellow solid, 179 mg, 99%) which was used without further purification.

### Synthesis of rac-(1S*,2S*)-2-(2,5-dichlorophenyl)cyclopropane-1-carboxylic acid

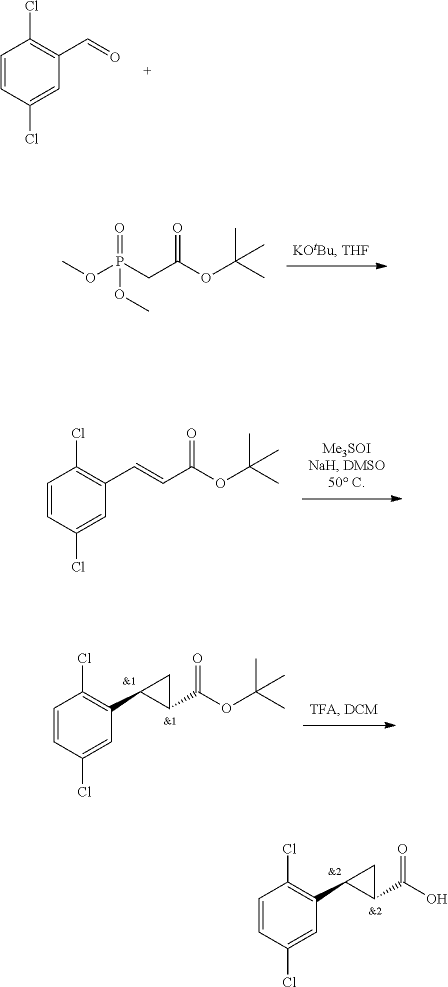 [SMILES: O=Cc1cc(Cl)ccc1Cl ; COP(=O)(CC(=O)OC(C)(C)C)OC ; CC(C)(C)OC(=O)/C=C/c1cc(Cl)ccc1Cl ; CC(C)(C)OC(=O)[C@H]1C[C@@H]1c1cc(Cl)ccc1Cl ; O=C(O)[C@H]1C[C@@H]1c1cc(Cl)ccc1Cl]

### Synthesis of tert-butyl (E)-3-(2,5-dichlorophenyl)acrylate

KO^{t}Bu (505 mg, 4.50 mmol) was added to a stirred solution of 2,5-dichlorobenzaldehyde (525 mg, 3.00 mmol) and tert-butyl 2-(dimethoxyphosphoryl)acetate (673 mg, 3.00 mmol) in THF (30 mL). The mixture was stirred at room temperature for 16 h, then diluted with EtOAc (30 mL). The solution was washed with water (30 mL) and brine (30 mL), then dried (hydrophobic frit) and concentrated in vacuo to give the title compound (840 mg, ˜90% purity, 96%) as a clear oil that was used in the next reaction without further purification. ¹H NMR (400 MHz, CDCl₃) δ 7.91 (d, J=17.1 Hz, 1H), 7.58 (d, J=2.4 Hz, 1H), 7.34 (d, J=8.5 Hz, 1H), 7.27 (d, J=2.9 Hz, 1H), 6.36 (d, J=15.0 Hz, 1H), 1.48 (s, 9H).

### Synthesis of rac-(1S*,2S*)-tert-butyl 2-(2,5-dichlorophenyl)cyclopropane-1-carboxylate

NaH (60% in mineral oil, 137 mg, 3.43 mmol) was added portion-wise to a solution of trimethylsulfoxonium iodide (754 mg, 3.43 mmol) in DMSO (5 mL). The resulting mixture was stirred for 30 min at room temperature followed by 1 h at 40° C., with regular sonication to assist solubilisation. A solution of tert-butyl (E)-3-(2,5-dichlorophenyl)acrylate (780 mg, 2.86 mmol) in DMSO (1 mL) was then added dropwise and the resulting mixture was stirred at 60° C. for 16 h. The mixture was diluted with water (25 mL) and extracted with EtOAc (2×10 mL), then the combined organics were washed with water and brine, dried (hydrophobic frit) and concentrated in vacuo. The resulting oil was purified by dry flash silica chromatography eluting with a gradient of 5-50% Et₂O in cyclohexane to give the desired product as an oil (466 mg, 56%). ¹H NM/R (400 MHz, CDCl₃) δ 7.29 (d, J=8.4 Hz, 1H), 7.13 (dd, J=2.4, 8.7 Hz, 1H), 6.97 (d, J=2.5 Hz, 1H), 2.63 (ddd, J=4.5, 6.7, 9.0 Hz, 1H), 1.75-1.70 (m, 1H), 1.59-1.54 (m, 1H), 1.48 (s, 9H), 1.27-1.22 (m, 1H).

### Synthesis of rac-(1S*,2S*)-2-(2,5-dichlorophenyl)cyclopropane-1-carboxylic acid

TFA (1.2 mL) was added to a solution of rac-(1S*,2S*)-tert-butyl 2-(2,5-dichlorophenyl)cyclopropane-1-carboxylate (450 mg, 1.57 mmol) in DCM (20 mL) and the mixture was stirred at room temperature for 16 h. The solvents were removed in vacuo to give the desired product as a grey solid (354 mg, 97%) which was used without further purification.

The compounds in Table A1 were synthesized using a similar method to rac-(1S*,2S*)-2-(2,5-dichlorophenyl)cyclopropane-1-carboxylic acid starting from the appropriate benzaldehyde derivatives:

TABLE Al

| Compound | Analytical data |
|---|---|
| 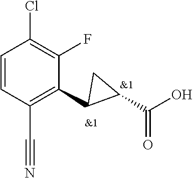 | compound used without purification |
| rac-(1S*,2S*)-2-(3-chloro-6-cyano-2- | |
| fluorophenyl)cyclopropane-1-carboxylic acid | |
| 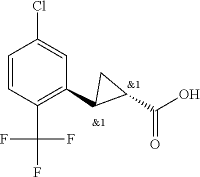 | ESI-MS (M + H)+: 263 |
| rac-(1S*,2S*)-2-(5-chloro-2- | |
| (trifluoromethyl)phenyl)cyclopropane-1-carboxylic | |
| acid | |
| 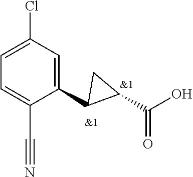 | ESI-MS (M + H)+: 220, ¹H NMR (400 MHz, DMSO) δ 12.53 − 12.52 (m, 1H), 7.86 (d, J = 7.9 Hz, 1H), 7.51 (d, J = 7.9 Hz, 1H), 7.38 (s, 1H), 2.69 − 2.64 (m, 1H), 1.59 − 1.53 (m, 2H). 1H obscured by DMSO signal. |
| rac-(1S*,2S*)-2-(5-chloro-2- | |
| cyanophenyl)cyclopropane-1-carboxylic acid | |
| 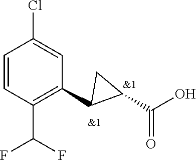 | ESI-MS (M + H)+: 245, ¹H NMR (400 MHz, DMSO) δ 7.62 (d, J = 8.1 Hz, 1H), 7.49 (d, J = 8.3 Hz, 1H), 7.46 − 7.17 (m, 2H), 2.70 − 2.64 (m, 1H), 1.99 − 1.92 (m, 1H), 1.60 − 1.40 (m, 2H). |
| rac-(1S*,2S*)-2-(5-chloro-2- | |
| (difluoromethyl)phenyl)cyclopropane-1-carboxylic | |
| acid | |
| 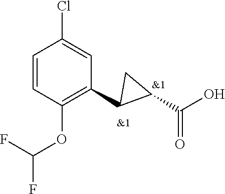 | ESI-MS (M + H)+: 261 |
| rac-(1S*,2S*)-2-(5-chloro-2- | |
| (difluoromethoxy)phenyl)cyclopropane-1- | |
| carboxylic acid | |
| 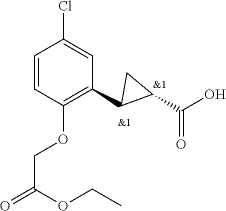 | ESI-MS (M + H)+: 297 |
| rac-(1S*,2S*)-2-(5-chloro-2-(2-ethoxy-2- | |
| oxoethoxy)phenyl)cyclopropane-1-carboxylic acid | |
| 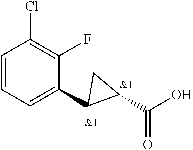 | ESI-MS (M + H)+: 213, ¹H NMR (400 MHz, CDCl₃) 7.26 (1H, s), 7.30 − 7.22 (1H, m), 7.00 (1H, dd, J = 8.0, 8.0 Hz), 6.89 (1H, dd, J-6.7, 6.7 Hz), 2.78 − 2.70 (1H, m), 1.98 − 1.91 (1H, m), 1.43 (1H, ddd, J = 4.6, 6.8, 8.3 Hz); |
| rac-(1S*,2S*)-2-(3-chloro-2- | |
| fluorophenyl)cyclopropane-1-carboxylic acid | |
| 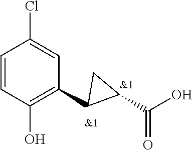 | ESI-MS (M + H)+: 211 |
| rac-(1S*,2S*)-2-(5-chloro-2- | |
| hydroxyphenyl)cyclopropane-1-carboxylic acid | |
| 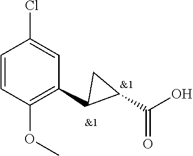 | ESI-MS (M + H)+: 227, ¹H NMR (400 MHz, CDCl₃) δ, 7.15 (dd, J = 2.3, 8.6 Hz, 1H), 6.87 (d, J = 2.3 Hz, 1H), 6.77 (d, J = 8.6 Hz, 1H), 3.84 (s, 3H), 2.76 (ddd, J = 4.0, 6.8, 9.1 Hz, 1H), 1.85 − 1.79 (m, 1H), 1.42 − 1.36 (m, 1H). |
| rac-(1S*,2S*)-2-(5-chloro-2- | |
| methoxyphenyl)cyclopropane-1-carboxylic acid | |

### Synthesis of rac-(1S*,2S*)-2-(5-chloro-2-hydroxyphenyl)cyclopropane-1-carboxamide

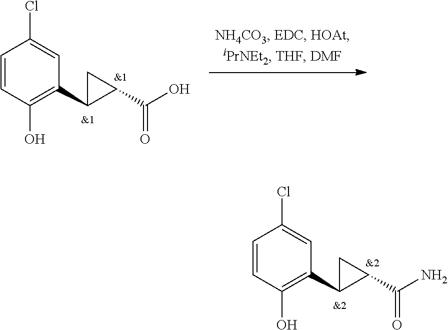 [SMILES: O=C(O)[C@H]1C[C@@H]1c1cc(Cl)ccc1O ; NC(=O)[C@H]1C[C@@H]1c1cc(Cl)ccc1O]

A solution of rac-(1S*,2S*)-2-(5-chloro-2-hydroxyphenyl)cyclopropane-1-carboxylic acid (100 mg, 0.47 mmol), EDC (108 mg, 0.56 mmol), HOAt (70 mg, 0.52 mmol), (NH₄)₂CO₃ (452 mg, 4.70 mmol) and DIPEA (0.37 mL, 2.12 mmol) in THF (4 mL) and DMF (4 mL) was stirred at 50° C. for 16 h. Water was added and the mixture was extracted with EtOAc. The organic extract was washed with brine and concentrated in vacuo. The crude product was purified by dry flash silica chromatography eluting with a gradient of 5-100% EtOAc in cyclohexane to give the desired product as a mixture of diastereoisomers (73 mg, 73%).

ESI-MS (M+H)−: 210, ¹H NMR (400 MHz, CDCl₃) δ, 6.99 (dd, J=2.3, 8.6 Hz, 1H), 6.82 (d, J=2.0 Hz, 1H), 6.77 (d, J=8.6 Hz, 1H), 6.45-6.36 (m, 1H), 6.00-5.89 (m, 1H), 2.60-2.51 (m, 1H), 1.70-1.61 (m, OH), 1.57-1.48 (m, 1H), 1.29-1.18 (m, 1H).

The compounds in Table A2 were synthesized using a similar method to rac-(1S*,2S*)-2-(5-chloro-2-hydroxyphenyl)cyclopropane-1-carboxamide starting from the appropriate carboxylic acids:

TABLE A2

| Compound | Coupling partner | Analytical Data |
|---|---|---|
| 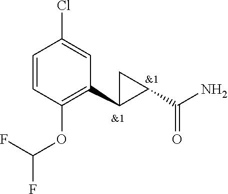 [SMILES: NC(=O)[C@H]1C[C@@H]1c1cc(Cl)ccc1OC(F)F] | rac-(1S*,2S*)-2-(5-chloro-2- (difluoromethoxy)phenyl)cyclo- propane-1-carboxylic acid | compound used without purification |
| rac-(1S*,2S*)-2-(5-chloro-2- | | |
| (difluoromethoxy)phenyl)cyclo- | | |
| propane-1-carboxamide | | |
| 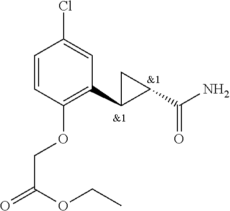 [SMILES: CCOC(=O)COc1ccc(Cl)cc1[C@H]1C[C@@H]1C(N)=O] | rac-(1S*,2S*)-2-(5-chloro-2- (2-ethoxy-2- oxoethoxy)phenyl)cyclo- propane-1-carboxylic acid | ESI-MS (M + H)+: 298, ¹H NMR (400 MHz, DMSO) δ 7.58-7.54 (m, 1H), 7.18 (dd, J = 2.6, 8.8 Hz, 1H), 6.95-6.89 (m, 3H), 4.85 (s, 2H), 4.18 (q, J = 7.1 Hz, 2H), 2.50-2.47 (m, 1H), 1.91-1.85 (m, 1H), 1.30- 1.28 (m, 1H), 1.26-1.20 (m, 4H). |
| rac-ethyl 2-(2-((1S*,2S*)-2- | | |
| carbamoylcyclopropyl)-4- | | |
| chlorophenoxy)acetate | | |

### Synthesis of (1S*,2S*)-2-(3-chloro-4-fluorophenyl)cyclopropanecarboxylic acid

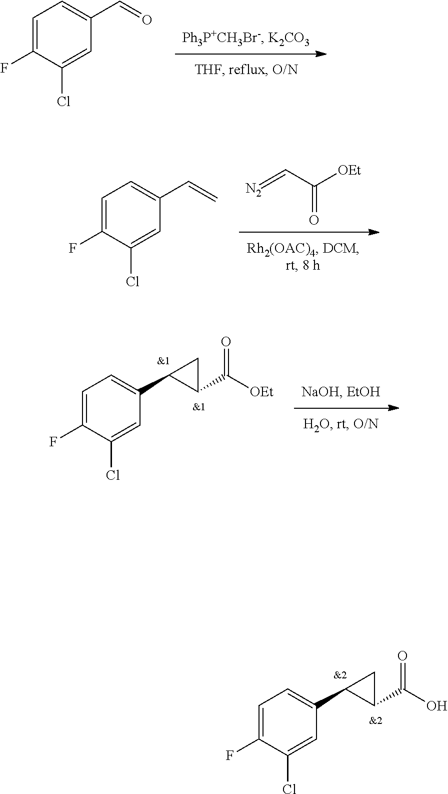

### Synthesis of 2-chloro-1-fluoro-4-vinylbenzene

5-chloro-2-fluorobenzaldehyde (5.0 g, 30 mmol) was added to a stirred suspension of potassium carbonate (8.0 g, 60 mmol) and methyltriphenylphonium bromide (17.0 g, 50 mmol) in anhydrous THF (150 mL) under nitrogen. After stirring at reflux overnight, the reaction mixture was cooled and filtered. The filtrate was concentrated in vacuo and the residue was purified by silica gel column chromatography (petroleum ether) to afford 2-chloro-1-fluoro-4-vinylbenzene (3.5 g, 71%) as a colourless oil.

¹H NMR (400 MHz, CDCl₃,) δ 7.43 (dd, J=7.2, 2.0 Hz, 1H), 7.27-7.23 (m, 1H), 7.08 (t, J=9.6 Hz, 1H), 6.44-6.57 (m, 1H), 5.68 (d, J=17.6 Hz, 1H), 5.27 (d, J=10.8 Hz, 1H).

### Synthesis of rac (1S*,2S*)-ethyl 2-(3-chloro-4-fluorophenyl)cyclopropanecarboxylate

To a suspension of 2-chloro-1-fluoro-4-vinylbenzene (1.0 g, 6.4 mmol) and diacetoxyrhodium (0.57 g, 1.3 mmol) in dichloromethane (40 mL) was added ethyl diazoacetate (4.4 g, 38.4 mmol) in DCM (20 mL) over 8 hours. After addition, the mixture was filtered, and the filtrate was concentrated in vacuo. The residue was purified by silica gel column chromatography (petroleum ether:ethyl acetate=10:1) to afford rac-(1S*,2S*)-ethyl 2-(3-chloro-4-fluorophenyl) cyclopropanecarboxylate (0.75 g, 48%) as a colourless oil.

1H NMR (400 MHz, CDCl₃,) δ 7.13 (dd, J=7.2, 2.0 Hz, 1H), 7.04 (t, J=8.8 Hz, 1H), 6.99-6.95 (m, 1H), 4.18 (q, J=7.2 Hz, 2H), 2.50-2.45 (m, 1H), 1.87-1.83 (m, 1H), 1.62-1.57 (m, 1H), 1.34-1.23 (m, 4H).

### Synthesis of rac (1S*,2S*)-2-(3-chloro-4-fluorophenyl)cyclopropanecarboxylic acid

To a solution of rac-(1S*,2S*)-ethyl 2-(3-chloro-4-fluorophenyl)cyclopropanecarboxylate (0.5 g, 2.1 mmol) in ethanol (40 mL) and water (10 mL) was added sodium hydroxide (0.3 g, 8.3 mmol). The mixture was stirred at room temperature overnight the concentrated in vacuo. The residue was diluted with water (20 mL), and acidified with 1N HCl to pH=1-2. The resulting precipitate was collected by filtration. The filter cake was dried in vacuum to afford (1S*,2S*)-2-(3-chloro-4-fluorophenyl)cyclopropanecarboxylic acid (0.3 g, 68%) as a white solid.

ESI-MS [M+H]+: 214.9, ¹H NMR (400 MHz, CDCl₃): δ 7.15 (dd, J=6.8 Hz, 2.0 Hz, 1H), 7.05 (t, J=8.8 Hz, 1H), 7.01-6.97 (m, 1H), 2.58-2.53 (m, 1H), 1.88-1.84 (m, 1H), 1.68-1.64 (m, 1H), 1.38-1.33 (m, 1H).

The compounds in Table A3 were synthesized using a similar method to rac (1S*,2S*)-2-(3-chloro-4-fluorophenyl)cyclopropanecarboxylic acid starting from the appropriate benzaldehyde derivatives:

TABLE A3

| Compound | Analytical data |
|---|---|
| 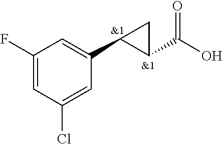 [SMILES: O=C(O)[C@H]1C[C@@H]1c1cc(F)cc(Cl)c1] | ESI-MS [M + H] +: 214.9, ¹H NMR (400 MHz, DMSO): δ 12.39 (s, 1H), 7.23 (dt, J = 8.8, 2.0 Hz, 1H), 7.19 (s, 1H), 7.07 (dt, J = 10.4, 2.0 Hz, 1H), 2.49-2.44 (m, 1H), 1.94- 1.90 (m, 1H), 1.46-1.39 (m, 2H). |
| (rac)-(1S*,2S*)-2-(3-chloro-5- | |
| fluorophenyl)cyclopropanecarboxylic acid | |
| 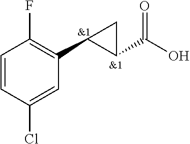 [SMILES: O=C(O)[C@H]1C[C@@H]1c1cc(Cl)ccc1F] | ESI-MS [M + H] +: 214.9, ¹H NMR (400 MHz, DMSO): δ 7.33-7.29 (m, 1H), 7.25-7.19 (m, 2H), 2.47-2.42 (m, 1H), 1.93-1.88 (m, 1H), 1.49-1.39 (m, 2H). |
| (rac)-(1S*,2S*)-2-(5-chloro-2- | |
| fluorophenyl)cyclopropanecarboxylic acid | |

### Synthesis of (1S,2S)-2-(4-methylpyrimidin-2-yl)cyclopropane-1-carboxamide

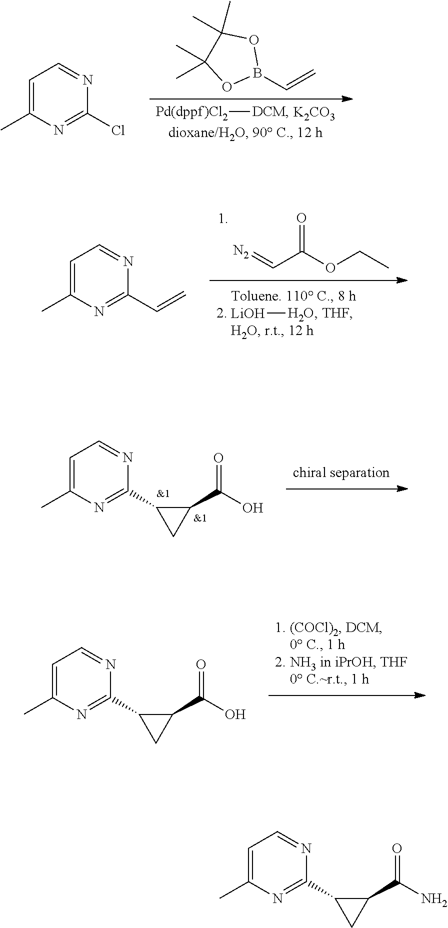

### Synthesis of 4-methyl-2-vinylpyrimidine

A mixture of 2-chloro-4-methylpyrimidine (6.4 g, 50 mmol), 4,4,5,5-tetramethyl-2-vinyl-1,3,2-dioxaborolane (10 g, 65 mmol), Pd(dppf)Cl₂-DCM (2 g, 2.5 mmol) and K₂CO₃ (17.25 g, 125 mmol) in dioxane (100 mL) and H₂O (5 ml) was stirred at 90° C. for 12 h. The reaction mixture was treated with H₂O (50 mL) and extracted with EA (50 ml×3). The combined organic layers were washed with brine (50 mL), dried over Na₂SO₄ and concentrated in vacuo (at 30° C.). The crude product was purified by silica column chromatography (PE/EtOAc=5/1) to give 4-methyl-2-vinylpyrimidine (4.8 g, 80%) as yellow oil. ESI-MS [M+H]⁺: 121.2.

### Synthesis of rac-ethyl (1S*, 2S*)-2-(4-methylpyrimidin-2-yl)cyclopropane-1-carboxylate

A solution of 4-methyl-2-vinylpyrimidine (4.8 g, 40 mmol) and ethyl 2-diazoacetate (9.12 g, 80 mmol) in toluene (70 mL) was refluxed at 110° C. for 8 h. The reaction was concentrated in vacuo and the crude product was purified by silica gel column (PE/EA=5/1) to give rac-ethyl (1S*, 2S*)-2-(4-methylpyrimidin-2-yl)cyclopropane-1-carboxylate (3.6 g, 43.7%) as yellow oil. ESI-MS [M+H]⁺: 207.2.

### Synthesis of rac-(1S*,2S*)-2-(4-methylpyrimidin-2-yl)cyclopropane-1-carboxylic acid

A mixture of rac-ethyl (1S*, 2S*)-2-(4-methylpyrimidin-2-yl)cyclopropane-1-carboxylate (3.6 g, 17.5 mmol) and LiOH—OH (1.4 g, 35 mmol) in THF/H₂O (20 mL/10 mL) was stirred at room temperature for 12 h. The reaction was concentrated in vacuo to remove THF and the pH of the residue was adjusted to 3 by HCl (2N). The white solid was precipitated, and mixture was filtered, dried to give rac-(1S*,2S*)-2-(4-methylpyrimidin-2-yl)cyclopropane-1-carboxylic acid (2.4 g, 77%) as a white solid. ESI-MS [M+H]⁺: 179.2. Purity: 95%. 1H NMR (400 MHz, DMSO) δ 12.45 (s, 1H), 8.51 (d, J=5.1 Hz, 1H), 7.20 (d, J=5.1 Hz, 1H), 2.58-2.52 (m, 1H), 2.41 (S, 3H), 2.07-2.03 (m, 1H), 1.54-1.45 (m, 2H).

### Separation of rac-(1S*,2S*)-2-(4-methylpyrimidin-2-yl)cyclopropane-1-carboxylic acid

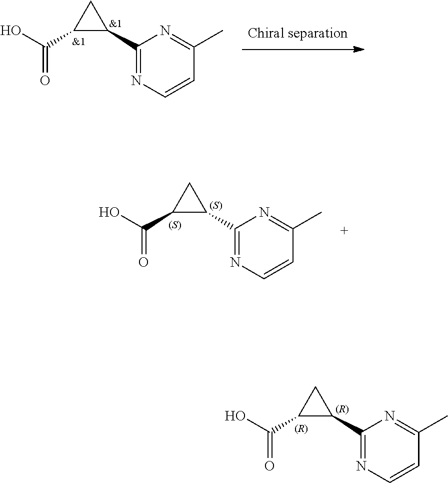

The mixture was separated using chiral column separation (Daicel CHIRALPAK AY, 250 mm L×50 mm I.D., 10 μm, EtOH/HOAc)=100/0.1 (V/V), 65 mL/min, 38° C.) to give two enantiomers: (1S,2S)-2-(4-methylpyrimidin-2-yl)cyclopropane-1-carboxylic acid and (1R,2R)-2-(4-methylpyrimidin-2-yl)cyclopropane-1-carboxylic acid

(1S,2S)-2-(4-methylpyrimidin-2-yl)cyclopropane-1-carboxylic acid (first eluting isomer): ESI-MS [M+H]+: 179.1. Purity: 95%. 1H NMR (400 MHz, DMSO) δ 12.45 (s, 1H), 8.51 (d, J=5.1 Hz, 1H), 7.20 (d, J=5.1 Hz, 1H), 2.57-2.52 (m, 1H), 2.41 (S, 3H), 2.07-2.03 (m, 1H), 1.53-1.46 (m, 2H). RT=4.6 min, 99% e.e.

(1R,2R)-2-(4-methylpyrimidin-2-yl)cyclopropane-1-carboxylic acid (second eluting isomer): ESI-MS [M+H]+: 179.1. Purity: 95%. 1H NMR (400 MHz, DMSO) δ 12.44 (s, 1H), 8.51 (d, J=5.1 Hz, 1H), 7.20 (d, J=5.1 Hz, 1H), 2.56-2.51 (m, 1H), 2.41 (S, 3H), 2.07-2.03 (m, 1H), 1.53-1.46 (m, 2H). RT=5.9 min, 99% e.e.

### Synthesis of (1S,2S)-2-(4-methylpyrimidin-2-yl)cyclopropane-1-carboxamide

To a solution of (1S, 2S)-2-(4-methylpyrimidin-2-yl)cyclopropane-1-carboxylic acid (1.1 g, 6.1 mmol) in dry DCM (20 mL) was added (COCl)₂ (1.56 g, 12.3 mmol) at 0° C. slowly and was stirred at 0° C. for another 1 h. The reaction mixture was concentrated in vacuo and the resulting acid chloride was dissolved in dry THF (20 mL), cooled to 0° C. and then added NH3 (20 mL, 2 M solution in iPrOH). The resulting solution was stirred at room temperature for another 1 h and concentrated in vacuo to give crude, which was purified with silica gel chromatography (eluent: DCM/MeOH=20/1) to furnish (1S,2S)-2-(4-methylpyrimidin-2-yl)cyclopropane-1-carboxamide (900 mg, 81.2%) as a yellow solid. ESI-MS [M+H]+: 178.1.

### Synthesis of rac-ethyl (1R*,2R*)-2-fluoro-2-(4-methylpyrimidin-2-yl)cyclopropane-1-carboxylate

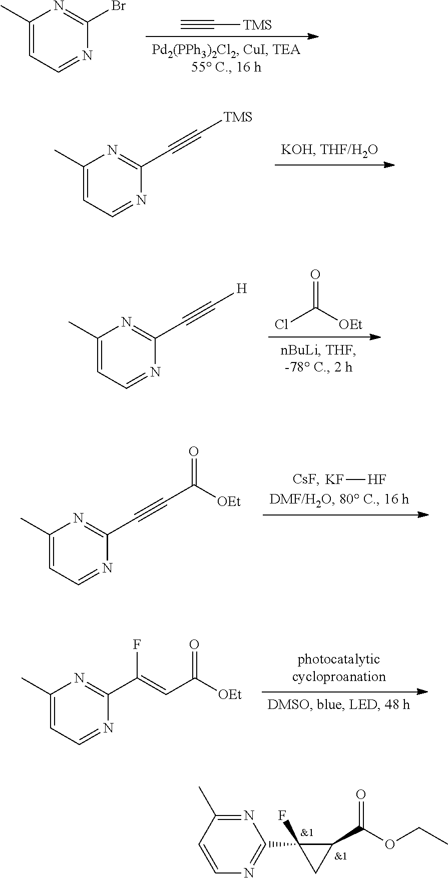

### Synthesis of 4-methyl-2-((trimethylsilyl)ethynyl)pyrimidine

To a solution of 2-bromo-4-methylpyrimidine (10 g, 58 mmol) in TEA (29.4 g, 290 mmol) were added ethynyltrimethylsilane (17.1 g, 174.4 mmol), Pd₂(PPh₃)₂Cl₂ (2.04 g, 2.9 mmol) and CuI (2.21 g, 11.6 mmol) under N₂ protection. The resulting reaction was stirred at 50° C. for 16 h, cooled to room temperature and concentrated to give the crude, which was purified by silica gel chromatography (EtOAc/PE=1/3) to give 4-methyl-2-((trimethylsilyl)ethynyl)pyrimidine (9 g, 82%) as a yellow solid. ESI-MS [M+H]⁺: 191.1.

### Synthesis of 2-ethynyl-4-methylpyrimidine

To a solution of 4-methyl-2-((trimethylsilyl)ethynyl)pyrimidine (9 g, 47.4 mmol) in THF (36 mL) was added KOH (8 g, 142 mmol) in water (140 mL). After stirring at room temperature for 2 h, the reaction was extracted with EtOAc/MeOH (10/1, 3×200 mL). The combined organics were washed by brine (200 mL), concentrated in vacuo and the residue purified by silica gel chromatography (EA/PE=1/1) to give 2-ethynyl-4-methylpyrimidine (1.5 g, 81%) as a yellow solid. ESI-MS [M+H]⁺: 119.1.

### Synthesis of ethyl 3-(4-methylpyrimidin-2-yl)propiolate

To a solution of 2-ethynyl-4-methylpyrimidine (1 g, 8.4 mmol) in THF (15 mL) was added n-BuLi (3.87 mL, 2.4M solution in hexane 9.3 mmol) at −78° C. under N₂ and the resulting mixture was stirred at −78° C. for 2 h. Ethyl carbonochloridate (3.8 g, 39.3 mmol) was added and the resulting mixture was stirred at −78° C. for another 1 h. The pH of reaction was adjusted to 7 with HCl (1N) and extracted with EtOAc (3×80 mL). The combined organic layers were washed with brine (60 mL), dried over Na₂SO₄ and concentrated in vacuo to give the crude, which was purified by silica gel chromatography (EtOAc/PE=1/4) to give ethyl 3-(4-methylpyrimidin-2-yl)propiolate (780 mg, 49%) as a white solid. ESI-MS [M+Na]⁺: 191.1

### Synthesis of ethyl (Z)-3-fluoro-3-(4-methylpyrimidin-2-yl)acrylate

To a solution of ethyl 3-(4-methylpyrimidin-2-yl)propiolate (780 mg, 4.1 mmol) in DMF/H₂O (15 mL/0.3 mL) was added KF-HF (350 mg, 4.5 mmol) and CsF (3.12 g, 20.5 mmol). The reaction mixture was stirred at 90° C. for 16 h. Water (60 mL) was added to the reaction and extracted with EtOAc (3×800 mL). The combined organic layers were washed with brine, dried over Na₂SO₄, concentrated in vacuo to give the crude, which was purified by silica gel chromatography (EtOAc/PE=1/5) to give ethyl (Z)-3-fluoro-3-(4-methylpyrimidin-2-yl)acrylate (370 mg, 43%) as colorless oil. ESI-MS [M+H]⁺: 211.1

### Synthesis of rac-ethyl (1R*,2R*)-2-fluoro-2-(4-methylpyrimidin-2-yl)cyclopropane-1-carboxylate

A flame dried 10 mL round bottom flask equipped with a magnetic stir bar was charged with ethyl (Z)-3-fluoro-3-(4-methylpyrimidin-2-yl)acrylate (370 mg, 1.76 mmol), 4CzIPN (2,4,5,6-tetra(9H-carbazol-9-yl)isophthalonitrile, 69 mg, 0.088 mmol), triethylammonium bis(catecholato)iodomethylsilicate (1.284 g, 2.647 mmol) and anhydrous DMSO (5 mL). The resulting solution was degassed (Vac/N₂, 3 cycles) and placed in front of two blue LEDs (Kessil, H150, 32 W). The lamps were arranged on both sides of the flask as close as possible to the surface. A fan for cooling was mounted over the setup. The reaction was left under irradiation for 48 h. The resulting reaction was poured into water (50 mL) and extracted with EtOAc:MeOH (10:1, 3×50 mL). The organic layers were washed with brine (50 mL), dried over Na₂SO₄, concentrated in vacuo and purified by silica gel chromatography (EtOAc/PE=1/4) to give rac-ethyl (1R*,2R*)-2-fluoro-2-(4-methylpyrimidin-2-yl)cyclopropane-1-carboxylate (140 mg, 35%) as yellow oil. ESI-MS [M+H]+: 225.2.

### Synthesis of rac-ethyl (1S*,2S*)-2-(5-fluoro-4-methylpyrimidin-2-yl)cyclopropane-1-carboxylate

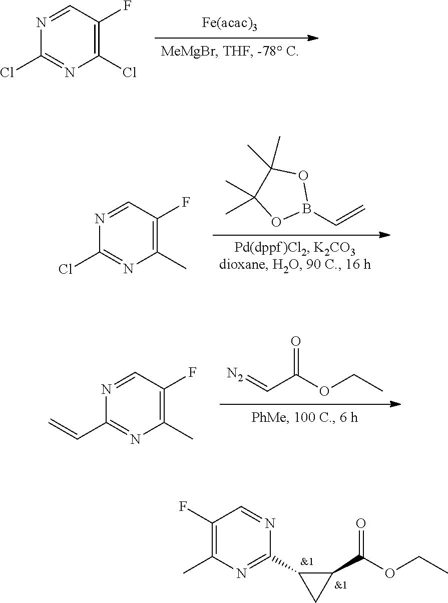

### Synthesis of 2-chloro-5-fluoro-4-methylpyrimidine

To a mixture of 2,4-dichloro-5-fluoropyrimidine (5 g, 30.1 mmol) in THF (80 mL) was added Fe(acac)₃ (1.17 g, 3.31 mmol). After cooling to −78° C., CH₃MgBr (3M solution in Et₂O, 17 mL, 51.5 mmol) was added dropwise. After stirring at −78° C. for 2 h under N₂, the reaction was quenched with saturated aqueous NH₄Cl (20 mL) and extracted with EtOAc (3×30 mL). The combined organic layers were washed with brine (30 mL), dried over anhydrous Na₂SO₄, and concentrated in vacuo to give the crude, which was purified by column chromatography (eluent: PE/EtOAc=15/1) to give 2-chloro-5-fluoro-4-methylpyrimidine (3.8 g, 86%) as a yellow oil. ESI-MS [M+H]+: No MS.

### Synthesis of 5-fluoro-4-methyl-2-vinylpyrimidine

To a mixture of 2-chloro-5-fluoro-4-methylpyrimidine (3.8 g, 26 mmol), 4,4,5,5-tetramethyl-2-vinyl-1,3,2-dioxaborolane (4.8 g, 31.2 mmol) and K₂CO₃ (10.8 g, 78 mmol) in dioxane/H₂O (80/20 mL) was added Pd(dppf)Cl₂ (952 mg, 1.3 mmol). The resulting reaction mixture was stirred at 90° C. for 16 h under N₂. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent: PE/EtOAc=15/1) to give 5-fluoro-4-methyl-2-vinylpyrimidine (2 g, 56%) as a yellow oil. ESI-MS [M+H]⁺: No MS.

### Synthesis of rac-ethyl (1S*,2S*)-2-(5-fluoro-4-methylpyrimidin-2-yl)cyclopropane-1-carboxylate

To a solution of 5-fluoro-4-methyl-2-vinylpyrimidine (500 mg, 3.62 mmol) in toluene (5 mL) was added ethyl 2-diazoacetate (1.24 g, 10.87 mmol). The resulting reaction mixture was stirred at 100° C. for 6 h under N₂, cooled to room temperature and then concentrated in vacuo to give the crude, which was purified by silica gel chromatography (eluent: PE/EtOAc=5/1) to give rac-ethyl (1S*,2S*)-2-(5-fluoro-4-methylpyrimidin-2-yl)cyclopropane-1-carboxylate (200 mg, 25%) as a yellow oil. ESI-MS [M+H]+: 225.2.

### Synthesis of rac-(1S*,2S*)-2-(4-methylthiazol-2-yl)cyclopropane-1-carboxamide

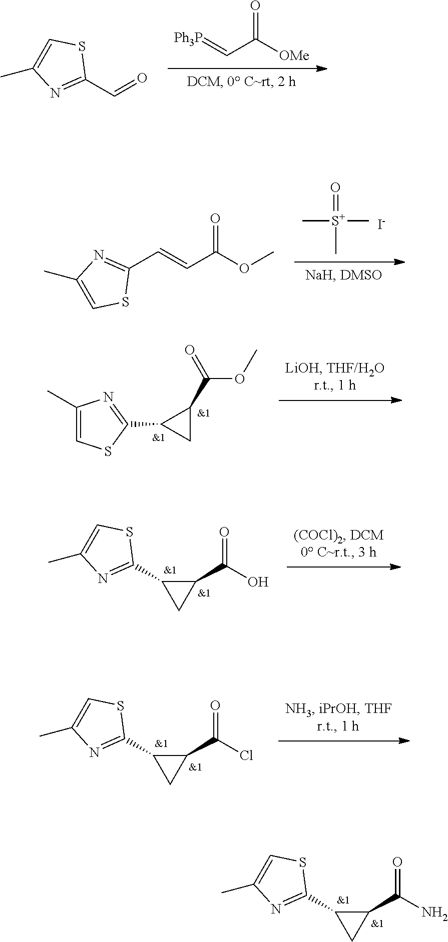

### Synthesis of methyl (E)-3-(4-methylthiazol-2-yl)acrylate

To a mixture of 4-methylthiazole-2-carbaldehyde (500 mg, 3.94 mmol) in dry DCM (20.0 mL) was added methyl 2-(triphenyl-15-phosphanylidene)acetate (1.32 g, 3.94 mmol) at 0° C. and the resulting reaction mixture was stirred at rt for 2 h. Water (50 mL) was added and the mixture was extracted with DCM (3×30 mL). The combined organic layers were washed with brine (50 mL), dried over anhydrous Na₂SO₄, and concentrated in vacuo to give the crude, which was purified by column chromatography (eluent: PE/EtOAc=10/1) to give methyl (E)-3-(4-methylthiazol-2-yl)acrylate (300 mg, 41.6%) as a yellow oil. ESI-MS [M+H]⁺: 184.2.

### Synthesis of rac-methyl (1S*,2S*)-2-(4-methylthiazol-2-yl)cyclopropane-1-carboxylate

A mixture of trimethylsulfoxonium iodide (0.726 g, 3.3 mmol) and NaH (132 mg, 60%, 3.3 mmol) in THF (25 mL) was stirred at room temperature for 40 min. Then a solution of ethyl (E)-3-(2-methylthiazol-4-yl)acrylate (200 mg, 1.1 mmol) in DMSO (5 mL) was added and stirred at 50° C. for 2 h. The mixture was quenched with saturated aqueous NaHCO₃ (30 mL) then extracted with EtOAc (3×30 mL). The combined organic layers were washed with brine (50 mL), dried over Na₂SO₄, concentrated in vacuo to give the crude product, which was purified with preparative TLC (eluent: PE/EtOAc=4/1) to give rac-methyl (1S*,2S*)-2-(4-methylthiazol-2-yl)cyclopropane-1-carboxylate (50 mg, 23%). ESI-MS [M+H]+: 198.2

### Synthesis of rac-(1S*,2S*)-2-(4-methylthiazol-2-yl)cyclopropane-1-carboxylic acid

A mixture of rac-methyl (1S*,2S*)-2-(4-methylthiazol-2-yl)cyclopropane-1-carboxylate (100 mg, 0.5 mmol) and LiOH—H₂O (63 mg, 1.5 mmol) in THF/H₂O (2 mL/2 mL) was stirred at room temperature for 1 h. The pH of the mixture was adjusted to 6 with HCl solution (4M, aq·) and then concentrated in vacuo to give rac-(1S*,2S*)-2-(4-methylthiazol-2-yl)cyclopropane-1-carboxylic acid as a white solid (150 mg, crude), which was used directly in the next step. ESI-MS [M+H]⁺: 184.1.

### Synthesis of rac-(1S*,2S*)-2-(4-methylthiazol-2-yl)cyclopropane-1-carbonyl chloride

To a mixture of rac-(1S*,2S*)-2-(4-methylthiazol-2-yl)cyclopropane-1-carboxylic acid (150 mg, crude) in DCM (5 mL) was added (COCl)₂ (192 mg, 1.52 mmol) at 0° C. The mixture was stirred for 3 h at room temperature and then concentrated in vacuo to give rac-(1S*,2S*)-2-(4-methylthiazol-2-yl)cyclopropane-1-carbonyl chloride (150 mg, crude), which was used directly in the next step. ESI-MS [M+H]⁺: 198.1.

### Synthesis of rac-(1S*,2S*)-2-(4-methylthiazol-2-yl)cyclopropane-1-carboxamide

To a solution of NH₃ in IPA (3 mL) was added a solution of rac-(1S*,2S*)-2-(4-methylthiazol-2-yl)cyclopropane-1-carbonyl chloride (150 mg, crude from previous step) in THF (1 mL) at 0° C. under N₂. The resulting mixture was allowed to warm to room temperature and stirred for 1 h then concentrated in vacuo and purified by preparative TLC (eluent: DCM/MeOH=10/1) to give rac-(1S*,2S*)-2-(4-methylthiazol-2-yl)cyclopropane-1-carboxamide as a white solid (60 mg, 66%). ESI-MS [M+H]⁺: 183.0.

### Synthesis of rac-ethyl (1S*,2S*)-2-(2-methylthiazol-4-yl)cyclopropane-1-carboxylate

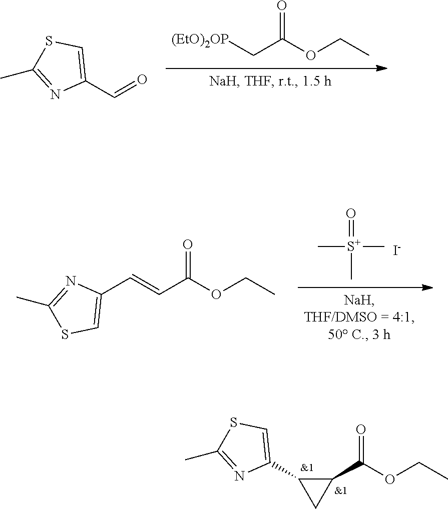

### Synthesis of ethyl (E)-3-(2-methylthiazol-4-yl)acrylate

To a mixture of NaH (202 mg, 60% suspension in paraffin oil, 5.06 mmol) in THF (20 mL) was added ethyl 2-(diethoxyphosphoryl)acetate (1.1 g, 5.06 mmol). The reaction mixture was stirred at room temperature for 0.5 h. Then a solution of 2-methylthiazole-4-carbaldehyde (586 mg, 4.6 mmol) in THF (5 mL) was added. The reaction was stirred at room temperature for another 1 h. The mixture was quenched with saturated aqueous NaHCO₃ (20 mL). The aqueous phase was extracted with EtOAc (3×30 mL). The combined organic layers were washed with brine (50 mL), dried over Na₂SO₄, concentrated in vacuo to give the crude product, which was purified with flash chromatography (eluent: PE/EtOAc=2/1) to give the product ethyl (E)-3-(2-methylthiazol-4-yl)acrylate as a white solid. (700 mg, 77%)

ESI-MS [M+H]+: 198.1

### Synthesis of rac-ethyl (1S*,2S*)-2-(2-methylthiazol-4-yl)cyclopropane-1-carboxylate

A mixture of trimethylsulfoxonium iodide (1.1 g, 2.25 mmol) and NaH (182 mg, 4.55 mmol) in THF (24 mL) was stirred at room temperature for 40 min. Then a solution of ethyl (E)-3-(2-methylthiazol-4-yl)acrylate (690 mg, 3.5 mmol) in DMSO (6 mL) was added and stirred at 50° C. for 2 h. The mixture was quenched with saturated aqueous NaHCO₃ (30 mL), extracted with EtOAc (3×30 mL). The combined organic layers were washed with brine (50 mL), dried over Na₂SO₄ then concentrated in vacuo to give the crude product, which was purified with preparative TLC (eluent: PE/EtOAc=4/1) to give rac-ethyl (1S*,2S*)-2-(2-methylthiazol-4-yl)cyclopropane-1-carboxylate as a colorless oil. (360 mg, 48%)

ESI-MS [M+H]+: 212.2

### Synthesis of (1R*,3R*)-2,2-difluoro-3-(4-methylpyrimidin-2-yl)cyclopropane-1-carboxylic acid

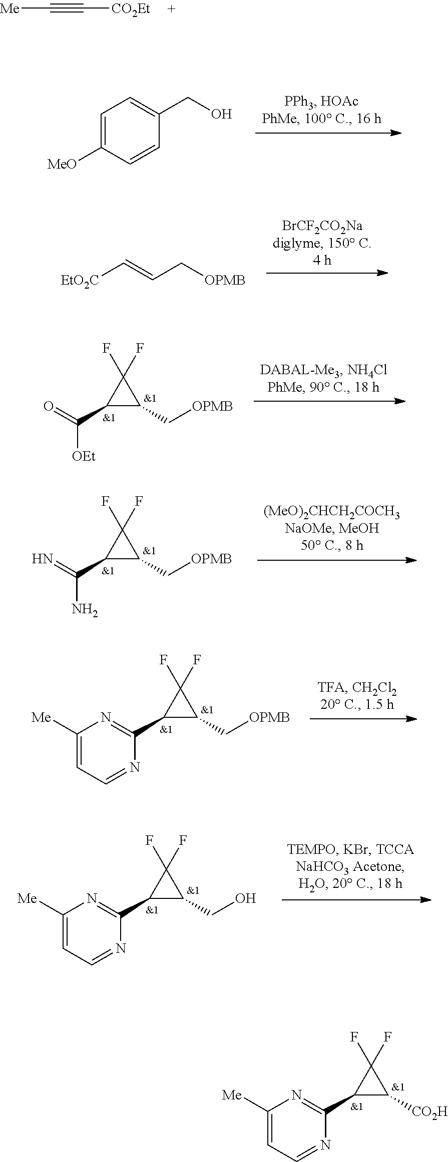

### Synthesis of ethyl (E)-4-((4-methoxybenzyl)oxy)but-2-enoate

A solution of ethyl tetrolate (7.22 g, 64.4 mmol), 4-methoxybenzyl alcohol (9.34 g, 67.6 mmol), triphenylphosphine (844 mg, 3.22 mmol) and acetic acid (736 μL, 12.9 mmol) in toluene (65 ml) was heated at 100° C. for 16 h. After being cooled to room temperature, the mixture was concentrated in vacuo and the resulting oil loaded onto a silica gel column (350 g) equilibrated with heptane:ethyl acetate=85:15. The column was eluted with this mixture (3 L) to obtain ethyl (E)-4-((4-methoxybenzyl)oxy)but-2-enoate as a colourless oil (11.92 g) which was used without further purification.

ESI-MS [M+Na] 273, ¹H NMR (400 MHz, CDCl₃, ppm) δ 7.27 (d, J 7 Hz, 2H), 6.97 (dt, J 16, 4 Hz, 1H), 6.89 (d, J 7 Hz, 2H), 6.11 (dt, J 16, 2 Hz, 1H), 4.49 (s, 2H), 4.20 (q, J 7 Hz, 2H), 4.15 (dd, J 4, 2 Hz, 2H), 3.81 (s, 3H), 1.29 (t, J 7 Hz, 3H).

### Synthesis of ethyl (1S*,3S*)-2,2-difluoro-3-(((4-methoxybenzyl)oxy)methyl)cyclopropane-1-carboxylate

Ethyl (E)-4-((4-methoxybenzyl)oxy)but-2-enoate (11.92 g, 47.6 mmol) was heated to 150° C. under nitrogen. A solution of sodium bromodifluoroacetate (28.1 g, 143 mmol) in anhydrous diglyme (48 mL) was added via syringe pump over 4 h with vigorous stirring. After the addition was complete the mixture was stirred for another 10 min and allowed to cool to room temperature. Heptane (300 mL) and water (250 mL) were added, the layers were separated, and the organic layer was passed through a sinter funnel to remove a small amount of precipitate. The aqueous layer was extracted with heptane (2×50 mL). The combined organic solutions were washed with water (3×250 mL), dried (MgSO₄), filtered and concentrated in vacuo to obtain crude (1S*,3S*)-2,2-difluoro-3-(((4-methoxybenzyl)oxy)methyl)cyclopropane-1-carboxylate as a brown oil.

¹H NMR (400 MHz, CDCl₃, ppm) δ 7.25 (d, J 9 Hz, 2H), 6.89 (d, J 9 Hz, 2H), 4.49 (d, J 12 Hz, 1H), 4.44 (d, J 12 Hz, 1H), 4.20 (q, J 7 Hz, 2H), 3.81 (s, 3H), 3.58 (d, J 7 Hz, 2H), 2.66-2.56 (m, 1H), 2.27 (dd, J 12, 8 Hz, 1H), 1.28 (t, J 7 Hz, 3H). ¹⁹F NMR (373 MHz, CDCl₃, ppm) δ −134.9 (ddd, J 155, 13, 1 Hz, 1F), −135.6 (dd, J 155, 13 Hz, 1F).

### Synthesis of (1S*,3S*)-2,2-difluoro-3-(((4-methoxybenzyl)oxy)methyl)cyclopropane-1-carboximidamide

An oven-dried 3-neck flask (250 mL) equipped with stir bar, reflux condenser and nitrogen inlet was charged with ammonium chloride (6.29 g, 118 mmol) and dry toluene (50 mL). The mixture was cooled to 0° C. and DABAL-Me₃ (30.2 g, 118 mmol) was added in one portion. The mixture was stirred for another 10 min and a solution of ethyl (1S*,3S*)-2,2-difluoro-3-(((4-methoxybenzyl)oxy)methyl)cyclopropane-1-carboxylate (7.07 g, 23.5 mmol) in dry toluene (66 mL) was added via syringe. The cooling bath was removed, and the mixture was heated to 90° C. (DrySyn) for 18 h. The mixture was cooled to room temperature and slowly poured into a stirred mixture of methanol:water=9:1 (400 mL) and Dicalite (100 g). After being stirred for another 20 min (until all effervescence had ceased), the solids were filtered. The resulting filter cake was washed with methanol (4×100 mL). The combined filtrates were concentrated under reduced pressure, and the resulting residue was co-evaporated with acetonitrile (3×200 mL) and then taken up in additional acetonitrile (100 mL). The solids were removed by filtration and washed with acetonitrile (3×25 mL). The combined filtrates were concentrated in vacuo to obtain crude (1S*,3S*)-2,2-difluoro-3-(((4-methoxybenzyl)oxy) methyl)cyclopropane-1-carboximidamide (ca 10 g), as a brown gum, which was used in the next step without further purification.

ESI-MS [M+H]⁺ 271.

### Synthesis of 2-((1S*,3S*)-2,2-difluoro-3-(((4-methoxybenzyl)oxy)methyl)cyclopropyl)-4-methylpyrimidine

1,1-Dimethoxypropan-2-one (3.42 mL, 28.2 mmol) and a solution of sodium methoxide (1 M in methanol, freshly prepared, 47 mL, 47 mmol) were added sequentially to a solution of crude (1S*,3S*)-2,2-difluoro-3-(((4-methoxybenzyl)oxy)methyl)cyclopropane-1-carboximidamide (˜10 g; assumed 23.5 mmol amidine) in anhydrous methanol (60 mL). The resulting solution was heated at 50° C. for 8 h. The mixture was concentrated in vacuo, and the residue was taken up in tert-butyl methyl ether (200 mL) and saturated aqueous sodium bicarbonate (150 ml). The layers were separated, and the aqueous layer was extracted with tert-butyl methyl ether (50 mL). The combined organic layers were washed with brine and dried (MgSO₄). The solution was filtered and concentrated in vacuo to obtain a brown oil, which was purified on a silica cartridge (Silicycle, Silaprep 220 g, 120 ml/min) using a gradient of heptane:ethyl acetate=7:3-55:45 (over 12 column volumes) to afford 2-((1S*,3S*)-2,2-difluoro-3-(((4-methoxybenzyl)oxy)methyl)cyclopropyl)-4-methylpyrimidine (4.44 g, 59% over 2 steps), as a light yellow oil.

ESI-MS [M+H]+ 321, ¹H NMR (400 MHz, CDCl₃, ppm) δ 8.50 (d, J 5 Hz, 1H), 7.27 (d, J 8 Hz, 2H), 7.02 (d, J 5 Hz, 1H), 6.88 (d, J 8 Hz, 2H), 4.54 (d, J 12 Hz, 1H), 4.47 (d, J 12 Hz, 1H), 3.80 (s, 3H), 3.76-3.67 (m, 2H), 3.05-2.95 (m, 1H), 2.86 (dd, J 14, 7 Hz, 1H), 2.50 (s, 3H).

¹⁹F NMR (373 MHz, CDCl₃, ppm) δ −133.0 (dd, J 155, 14 Hz, 1F), −139.2 (ddd, J 155, 14, 3 Hz, 1F).

### Synthesis of ((1S*,3S*)-2,2-difluoro-3-(4-methylpyrimidin-2-yl)cyclopropyl)methanol

2-((1S*,3S*)-2,2-Difluoro-3-(((4-methoxybenzyl)oxy)methyl)cyclopropyl)-4-methylpyrimidine (710 mg, 2.22 mmol) was dissolved in DCM (40.5 mL), and trifluoroacetic acid (4.0 mL, 52.2 mmol) was added followed by water (500 μL, 27.8 mmol). The reaction mixture was stirred at room temperature for 1.5 h. The mixture was neutralised (to pH7) by the addition of solid sodium bicarbonate and water (45 mL). The aqueous layer was separated and extracted with DCM (3×45 mL). The organic solutions were combined, washed with water (45 mL), dried over Na₂SO₄ and concentrated in vacuo to give a red solid. Purification by flash chromatography (loading the sample in solution in ethyl acetate, Silicycle, Silaprep 25 g cartridge), eluting with a gradient of heptane:ethyl acetate=90:10-0:100, then ethyl acetate:methanol=100:0-80:20) to afford ((1S*,3S*)-2,2-difluoro-3-(4-methylpyrimidin-2-yl)cyclopropyl)methanol (353 mg, yield 80%), as a colourless solid.

ESI-MS [M+H]+ 201, ¹H NMR (396 MHz, CDCl₃) δ 8.51 (d, J 5.4 Hz, 1H), 7.04 (d, J 5.4 Hz, 1H), 3.98-3.87 (m, 2H), 3.04-2.90 (m, 2H), 2.51 (s, 3H).

¹⁹F NMR (373 MHz, CDCl₃) δ −133.8-−134.3 (m, 1F), −138.7 (ddd, J 157, 13, 2 Hz, 1F).

### Synthesis of (1S*,3S*)-2,2-difluoro-3-(4-methylpyrimidin-2-yl)cyclopropane-1-carboxylic acid

((1S*,3S*)-2,2-Difluoro-3-(4-methylpyrimidin-2-yl)cyclopropyl)methanol (590 mg, 2.95 mmol) was dissolve in acetone (30 mL) and saturated aqueous sodium bicarbonate (8.7 mL) was added to obtain a cloudy solution. The solution was cooled to 0° C. in an ice bath, and potassium bromide (71 mg, 0.60 mmol) and (2,2,6,6-tetramethylpiperidin-1-yl)oxyl (TEMPO, 9 mg, 0.058 mmol) were added. Trichloroisocyanuric acid (1.378 g, 5.93 mmol) was added in small portions over 10 minutes. The reaction mixture was allowed to reach room temperature and stirred at room temperature for 18 h. The reaction was quenched by the addition of isopropanol (2 mL) and acetone (30 mL), and the mixture was filtered. The filtrate was concentrated in vacuo to remove acetone and isopropanol. The resulting aqueous solution was diluted with water, filtered again, and the solid was washed with DCM (30 mL). The biphasic filtrate was concentrated in vacuo to remove the DCM. The aqueous solution (pH 7) was rendered basic (pH 11) by the addition of aqueous sodium hydroxide (4M). The aqueous solution was washed with DCM (3×15 mL), neutralised (pH 7) by adding hydrochloric acid (2M), and then washed again with DCM (3×15 mL). Finally, the aqueous solution was acidified (pH 1) by adding hydrochloric acid (2M) and the product was extracted into DCM (4×15 mL). The pH of the aqueous layer was found to be ˜2.5, therefore, more hydrochloric acid (2M) was added and further extractions with DCM (4×15 mL) were performed. The combined organics were concentrated in vacuo to obtain (1S*,3S*)-2,2-difluoro-3-(4-methylpyrimidin-2-yl)cyclopropane-1-carboxylic acid (447 mg, 71%), as a pale pink solid.

ESI-MS [M+H]⁺ 215, ¹H NMR (396 MHz, CDCl₃): δ 8.59 (d, J 5.4 Hz, 1H, H-2′), 7.13 (d, J 5.4 Hz, 1H, H-3′), 3.79 (ddd, J 13, 7.7, 1.4 Hz, 1H, H-2), 3.51 (dd, J 13, 7.7 Hz, 1H, H-3), 2.55 (s, 3H, H-5′).

¹⁹F NMR (373 MHz, CDCl₃) δ −130.8 (dd, J 150, 13 Hz, 1F), −135.3-−135.7 (m, 1F).

### Synthesis of rac-methyl (1S*,3S*)-2,2-difluoro-3-(4-methylpyrimidin-2-yl)cyclopropane-1-carboxylate

To a mixture of rac-(1S*,3S*)-2,2-difluoro-3-(4-methylpyrimidin-2-yl)cyclopropane-1-carboxylic acid ((150 mg, 0.7 mmol) in CH₃OH (10 mL) was added SOCl₂ (167 mg, 1.7 mmol). The reaction mixture was stirred at 40° C. for 3 h. The reaction mixture was poured into water (50 mL) then extracted with EtOAc (3×50 mL). The combined organics were washed with brine (50 mL), dried over Na₂SO₄, concentrated in vacuo to give the crude, which was purified by flash chromatography (PE/EtOAc=5/1) to get rac-methyl (1S*,3S*)-2,2-difluoro-3-(4-methylpyrimidin-2-yl)cyclopropane-1-carboxylate as colorless oil (120 mg, yield 75%). ESI-MS [M+H]+: 229

### Synthesis of rac-ethyl (1S*,2S*)-2-(4-methyl-1,3,5-triazin-2-yl)cyclopropane-1-carboxylate

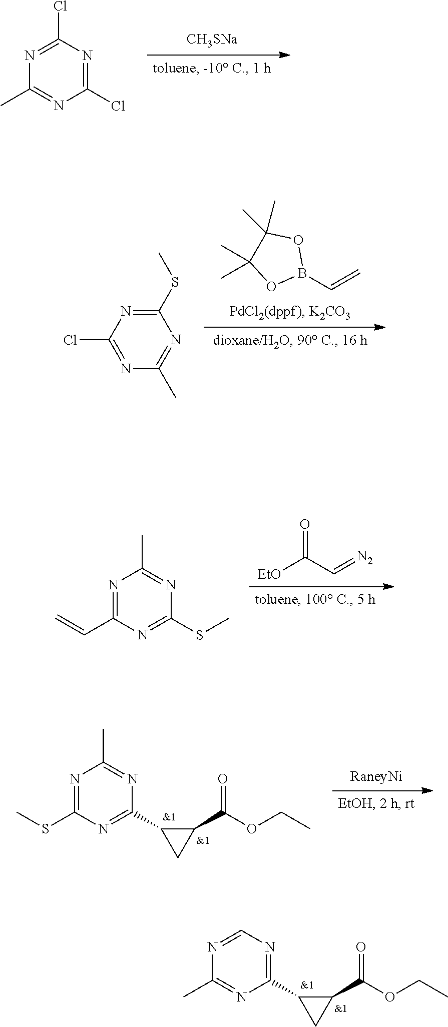

### Synthesis of 2-chloro-4-methyl-6-(methylthio)-1,3,5-triazine

To a solution of 2,4-dichloro-6-methyl-1,3,5-triazine (2 g, 12.2 mmol) in toluene (20 mL) was added CH₃SNa (769 mg, 10.9 mmol) slowly at −10° C. The mixture was stirred for 1 h at −10° C. then water (20 mL) was added. The mixture was extracted with EtOAc (3×30 mL). The combined organic layers were washed with brine (20 mL), dried over Na₂SO₄ then concentrated in vacuo to give the crude, which was purified by column chromatography (PE:EtAOc=3:1) to give 2-chloro-4-methyl-6-(methylthio)-1,3,5-triazine (1 g, 47.6%) as a white solid. ESI-MS [M+H]⁺: 176.0.

### Synthesis of 2-methyl-4-(methylthio)-6-vinyl-1,3,5-triazine

To a mixture of benzyl 2-chloro-4-methyl-6-(methylthio)-1,3,5-triazine (495 mg, 2.81 mmol) in dioxane (15 mL) and water (1.5 mL) was added 4,4,5,5-tetramethyl-2-vinyl-1,3,2-dioxaborolane (1.2 g, 3.15 mmol), PdCl₂(dppf) (206 mg, 0.28 mmol) and K₂CO₃ (1.16 g, 8.43 mmol) at 25° C. The resulting reaction mixture was then stirred at 90° C. for 16 h then cooled to room temperature. The reaction mixture was filtered through Celite® and the filter cake was washed with DCM/MeOH (10/1, 50 mL). The filtrate was concentrated in vacuo to give the crude, which was purified by column chromatography (eluent: PE/EtAOc=20/1˜10/1) to give 2-methyl-4-(methylthio)-6-vinyl-1,3,5-triazine (250 mg, 53.2%) as yellow oil. ESI-MS [M+H]+: 168.0.

### Synthesis of rac-ethyl (1S*, 2S*)-2-(4-methyl-6-(methylthio)-1,3,5-triazin-2-yl)cyclopropane-1-carboxylate

To a mixture of 2-methyl-4-(methylthio)-6-vinyl-1,3,5-triazine (250 mg, 1.5 mmol) in toluene (15 mL) was added ethyl 2-diazoacetate (512 mg, 4.5 mmol). The resulting reaction mixture was stirred at 100° C. for 5 h under a N₂ atmosphere. The reaction was cooled to room temperature then filtered. The filtrate was concentrated to give the crude product, which was purified by silica column chromatography (eluent: PE/EtAOc=3/1) to give rac-ethyl (1S*, 2S*)-2-(4-methyl-6-(methylthio)-1,3,5-triazin-2-yl)cyclopropane-1-carboxylate (100 mg, 26.3%) as a yellow solid. ESI-MS [M+H]⁺: 254.1.

### Synthesis of rac-ethyl (1S*,2S*)-2-(4-methyl-1,3,5-triazin-2-yl)cyclopropane-1-carboxylate

A mixture of rac-ethyl (1S*,2S*)-2-(4-methyl-6-(methylthio)-1,3,5-triazin-2-yl)cyclopropane-1-carboxylate (100 mg, 0.4 mmol) and RaneyNi (100 mg) in EtOH (3 mL) was stirred at room temperature for 2 h. The reaction mixture was filtered, the filtrate was concentrated in vacuo to give rac-ethyl (1S*,2S*)-2-(4-methyl-1,3,5-triazin-2-yl)cyclopropane-1-carboxylate (60 mg, 72.4%) as colorless oil, which was used into next step without further purification. ESI-MS [M+H]⁺: 208.1.

### Synthesis of rac-ethyl (1S*,2S*)-2-(4,6-dimethylpyrimidin-2-yl)cyclopropane-1-carboxylate

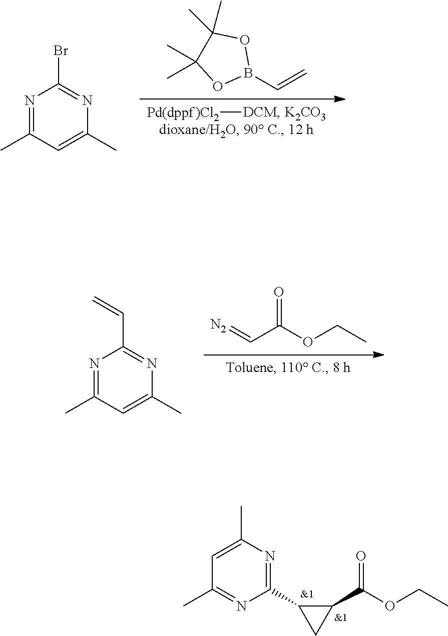

### Synthesis of 4,6-dimethyl-2-vinylpyrimidine

A mixture of 2-bromo-4,6-dimethylpyrimidine (900 mg, 4.8 mmol), 4,4,5,5-tetramethyl-2-vinyl-1,3,2-dioxaborolane (875 mg, 5.7 mmol), Pd(dppf)Cl₂-DCM (192 mg, 0.23 mmol) and K₂CO₃ (1.9 g, 14.1 mmol) in dioxane (30 mL) and water (3 ml) was stirred at 90° C. for 12 h. Water (30 mL) was added to the reaction, extracted with EA (3×50 mL). The combined organic layers were washed with brine (50 mL), dried over Na₂SO₄, concentrated in vacuo to give the crude, which was purified by silica column chromatography (PE/EtOAc=5/1) to give 5-(prop-1-en-2-yl)-2,3-dihydro-1H-inden-4-amine (500 mg, 78%) as a yellow solid. ESI-MS [M+H]⁺: 135.2.

### Synthesis of rac-ethyl (1S*,2S*)-2-(4,6-dimethylpyrimidin-2-yl)cyclopropane-1-carboxylate

A mixture of 4,6-dimethyl-2-vinylpyrimidine (500 mg, 3.7 mmol) and ethyl 2-diazoacetate (843 mg, 7.4 mmol) in toluene (30 mL) was heated at 110° C. for 8 h. The reaction was cooled to room temperature and concentrated in vacuo to give the crude, which was purified by silica gel column (PE/EtOAc=5/1) to give rac-ethyl (1S*,2S*)-2-(4,6-dimethylpyrimidin-2-yl)cyclopropane-1-carboxylate (250 mg, 31%) as a yellow solid. ESI-MS [M+H]⁺: 221.2.

The compounds in Table A4 were synthesized using a similar method to rac-ethyl (1S*,2S*)-2-(4,6-dimethylpyrimidin-2-yl)cyclopropane-1-carboxylate starting from the appropriate heteroaryl bromide or chloride derivatives:

TABLE A4

| Compound | Analytical data |
|---|---|
| 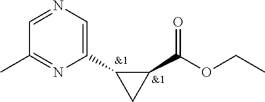 | ESI-MS [M + H]⁺: 207.1 |
| rac-ethyl (1S*,2S*)-2-(6-methylpyrazin-2- | |
| yl)cyclopropane-1-carboxylate | |
| 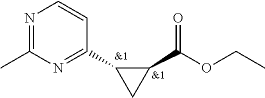 | ESI-MS [M + H] ⁺: 207.1 |
| rac-ethyl (1S*,2S*)-2-(2-methylpyrimidin-4- | |
| yl)cyclopropane-1-carboxylate | |
| 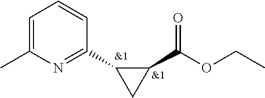 | ESI-MS [M + H ]⁺: 206.1 |
| rac-ethyl (1S*,2S*)-2-(6-methylpyridin-2- | |
| yl)cyclopropane-1-carboxylate | |
| 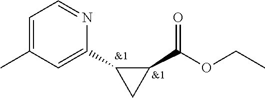 | ESI-MS [M + H ]⁺: 206.1 |
| rac-ethyl (1S*,2S*)-2-(4-methylpyridin-2- | |
| yl)cyclopropane-1-carboxylate | |
| 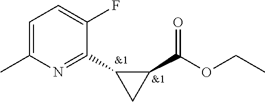 [SMILES: CCOC(=O)[C@H]1C[C@@H]1c1nc(C)ccc1F] | ESI-MS [M + H] ⁺: 224.1 |
| rac-ethyl (1S*,2S*)-2-(3-fluoro-6-methylpyridin- | |
| 2-yl)cyclopropane-1-carboxylate | |
| 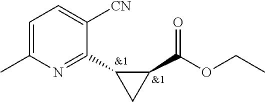 [SMILES: CCOC(=O)[C@H]1C[C@@H]1c1nc(C)ccc1C#N] | ESI-MS [M + H]⁺: 231.1 |
| rac-ethyl (1S*,2S*)-2-(3-cyano-6-methylpyridin-2- | |
| yl)cyclopropane-1-carboxylate | |
| 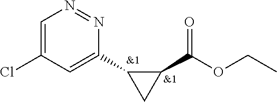 | ESI-MS [M + H]⁺: 227.2 |
| rac-ethyl (1S*,2S*)-2-(5-chloropyridazin-3- | |
| yl)cyclopropane-1-carboxylate | |
| 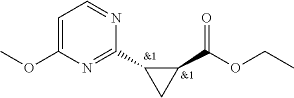 [SMILES: CCOC(=O)[C@H]1C[C@@H]1c1nccc(OC)n1] | ESI-MS [M + H]⁺: 223.1 |
| rac-ethyl (1S*,2S*)-2-(4-methoxypyrimidin-2- | |
| yl)cyclopropane-1-carboxylate | |
| 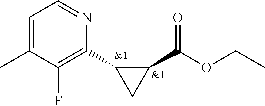 | ESI-MS [M + H]⁺: 244.2 |
| rac-ethyl (1S*,2S*)-2-(3-fluoro-4-methylpyridin-2- | |
| yl)cyclopropane-1-carboxylate | |

### Synthesis of rac-(1S*,2S*)-2-(4-(fluoromethyl)pyrimidin-2-yl)cyclopropane-1-carboxamide

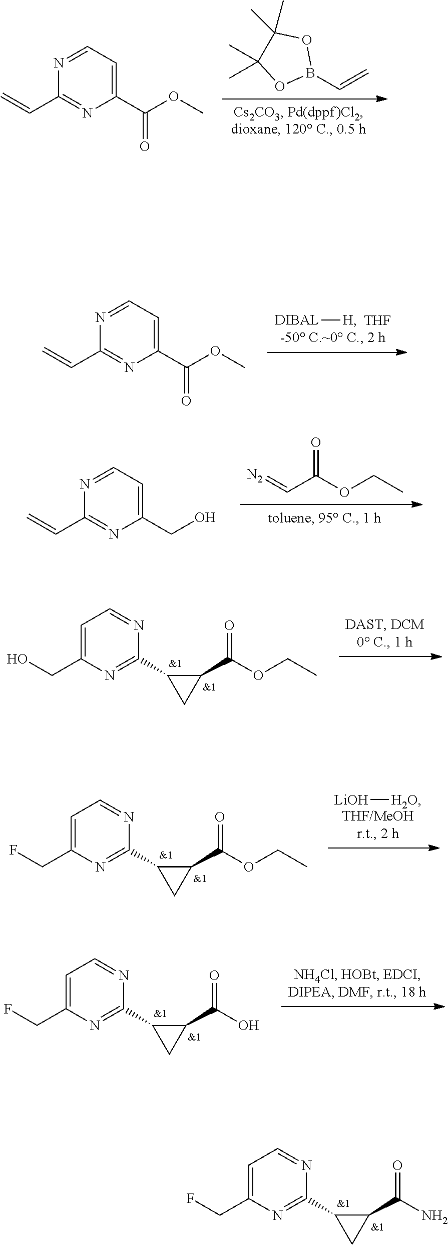

### Synthesis of methyl 2-vinylpyrimidine-4-carboxylate

To a mixture of methyl 2-chloropyrimidine-4-carboxylate (1.2 g, 6.97 mmol), 4,4,5,5-tetramethyl-2-vinyl-1,3,2-dioxaborolane (1.61 g, 10.4 mmol) and Cs₂CO₃ (6.8 g, 20.9 mmol) in dioxane/H₂O (20/5 mL) was added Pd(dppf)Cl₂ (254.9 mg, 0.35 mmol). The resulting reaction mixture was stirred at 120° C. for 5 h under N₂. After cooling to room temperature, water (50 mL) was added and the mixture was extracted with EtOAc (3×50 mL). The combined organic layers were washed with brine (60 mL), dried over anhydrous Na₂SO₄, and concentrated in vacuo to give the crude, which was purified by column chromatography (eluent: PE/EtOAc=15/1) to give methyl 2-vinylpyrimidine-4-carboxylate (700 mg, 63.6%) as a yellow oil. ESI-MS [M+H]⁺: 165.2.

### Synthesis of (2-vinylpyrimidin-4-yl)methanol

To a solution of methyl 2-vinylpyrimidine-4-carboxylate (300 mg, 1.8 mmol) in THF (6 mL) was added DIBAL-H (4.57 ml, 1.0M in hexane) at −50° C. The mixture was stirred at 0° C. for 2 h, water (10 mL) was added and the resulting mixture was extracted with EtOAc (20 mL×3). The combined organic layers were washed with brine (30 mL), dried over Na₂SO₄, filtered and concentrated in vacuo to give the crude, which was purified by preparative TLC(PE/EA=5/1) to give (2-vinylpyrimidin-4-yl)methanol (180 mg, 73%) as a yellow solid. ESI-MS [M+H]+: 137.2.

### Synthesis of rac-ethyl (1S*,2S*)-2-(4-(hydroxymethyl)pyrimidin-2-yl)cyclopropane-1-carboxylate

To a solution of (2-vinylpyrimidin-4-yl)methanol (180 mg, 1.3 mmol) in toluene (5 mL) was added ethyl 2-diazoacetate (444.7 mg, 3.97 mmol). The resulting reaction mixture was stirred at 95° C. for 1 h under N₂. The reaction was then cooled to room temperature and concentrated in vacuo to give the crude, which was purified by preparative TLC (eluent: PE/EtOAc=3/1) to give rac-ethyl (1S*,2S*)-2-(4-(hydroxymethyl)pyrimidin-2-yl)cyclopropane-1-carboxylate (100 mg, 34.6%) as a yellow oil. ESI-MS [M+H]+: 223.2.

### Synthesis of rac-ethyl (1S*,2S*)-2-(4-(fluoromethyl)pyrimidin-2-yl)cyclopropane-1-carboxylate

To a solution of rac-ethyl (1S*,2S*)-2-(4-(hydroxymethyl)pyrimidin-2-yl)cyclopropane-1-carboxylate (100 mg, 0.45 mmol) in DCM (3 mL) was added DAST (93.3 mg, 0.58 mmol) at 0° C., After stirring the resulting reaction mixture was stirred at 0° C. for 1 h under N₂, water (20 mL) was added and extracted with EtOAc (20 mL×3). The combined organic layers were washed with brine (20 mL), dried over Na₂SO₄, filtered and concentrated in vacuo to give the crude, which was purified by preparative TLC (PE/EtOAc=5/1) to give rac-ethyl (1S*,2S*)-2-(4-(fluoromethyl)pyrimidin-2-yl)cyclopropane-1-carboxylate (60 mg, 60%) as a yellow oil. ESI-MS [M+H]+: 225.2.

### Synthesis of rac-(1S*,2S*)-2-(4-(fluoromethyl)pyrimidin-2-yl)cyclopropane-1-carboxamide

Using a similar procedure to that for 2-(5-chloro-2-nitrophenyl)cyclopropane-1-carboxamide, rac-(1S*,2S*)-2-(4-(fluoromethyl)pyrimidin-2-yl)cyclopropane-1-carboxamide (30 mg, 60%) was synthesised from rac-ethyl (1S*,2S*)-2-(4-(fluoromethyl)pyrimidin-2-yl)cyclopropane-1-carboxylate (60 mg, 0.26 mmol). ESI-MS [M+H]+: 196.2.

### Synthesis of rac-(1S*, 2S*)-2-(4-(difluoromethyl)pyrimidin-2-yl)cyclopropane-1-carboxamide

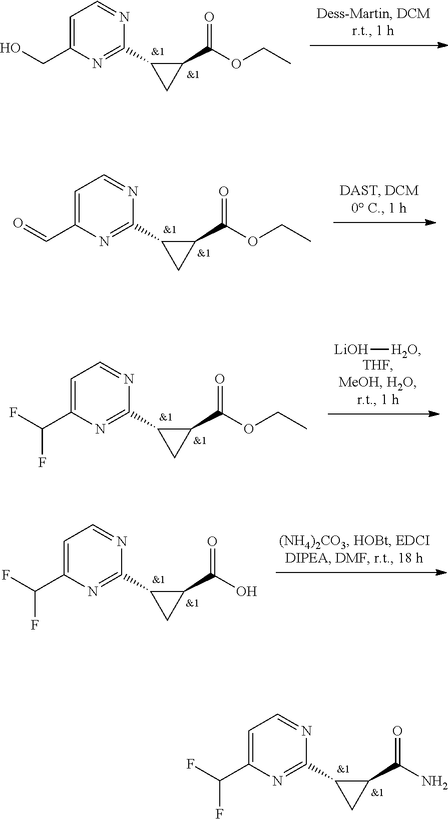

### Synthesis of rac-ethyl (1S*, 2S*)-2-(4 formylpyrimidin-2-yl)cyclopropane-1-carboxylate

To a solution of rac-ethyl (1S*, 2S*)-2-(4-(hydroxymethyl)pyrimidin-2-yl)cyclopropane-1-carboxylate (200 mg, 0.9 mmol) in DCM (5 mL) was added Dess-Martin periodinane (457.9 mg, 1.08 mmol). The resulting reaction mixture was stirred at room temperature for 1 h under N₂. The reaction was quenched with saturated aqueous NH₄Cl (15 mL) and extracted with DCM (3×20 mL). The combined organic layers were washed with brine (30 mL), dried over Na₂SO₄, concentrated in vacuo to give the crude, which was purified by preparative TLC (eluent: PE/EtOAc=5/1) to give rac-ethyl (1S*,2S*)-2-(4-formylpyrimidin-2-yl)cyclopropane-1-carboxylate (170 mg, 85%) as a yellow solid. ESI-MS [M+H]+: 221.2.

### Synthesis of rac-ethyl (1S*, 2S*)-2-(4-(difluoromethyl)pyrimidin-2-yl)cyclopropane-1-carboxylate

To a solution of rac-ethyl (1S*, 2S*)-2-(4-formylpyrimidin-2-yl)cyclopropane-1-carboxylate (170 mg, 0.77 mmol) in DCM (3 mL) was added DAST (149.5 mg, 0.93 mmol) at 0° C., After stirring the resulting reaction mixture at 0° C. for 1 h under N₂, water (20 mL) was added to the reaction, extracted with EtOAc (3×20 mL). The combined organic layers were washed with brine (30 mL), dried over Na₂SO₄, filtered and concentrated in vacuo to give the crude, which was purified by preparative TLC (PE/EtOAc=5/1) to give rac-ethyl (1S*, 2S*)-2-(4-(difluoromethyl)pyrimidin-2-yl)cyclopropane-1-carboxylate (100 mg, 53.4%) as a yellow oil. ESI-MS [M+H]+: 243.2.

### Synthesis of rac-(1S*, 2S*)-2-(4-(difluoromethyl)pyrimidin-2-yl)cyclopropane-1-carboxamide

Using a similar procedure to that for 2-(5-chloro-2-nitrophenyl)cyclopropane-1-carboxamide, rac-(1S*, 2S*)-2-(4-(difluoromethyl)pyrimidin-2-yl)cyclopropane-1-carboxamide (13 mg, 50%) was synthesised from rac-ethyl (1S*, 2S*)-2-(4-(difluoromethyl)pyrimidin-2-yl)cyclopropane-1-carboxylate (100 mg, 0.41 mmol). ESI-MS [M+H]+: 196.2.

### Synthesis of rac-(1S*,2S*)-2-(4-(trifluoromethyl)pyrimidin-2-yl)cyclopropane-1-carboxamide

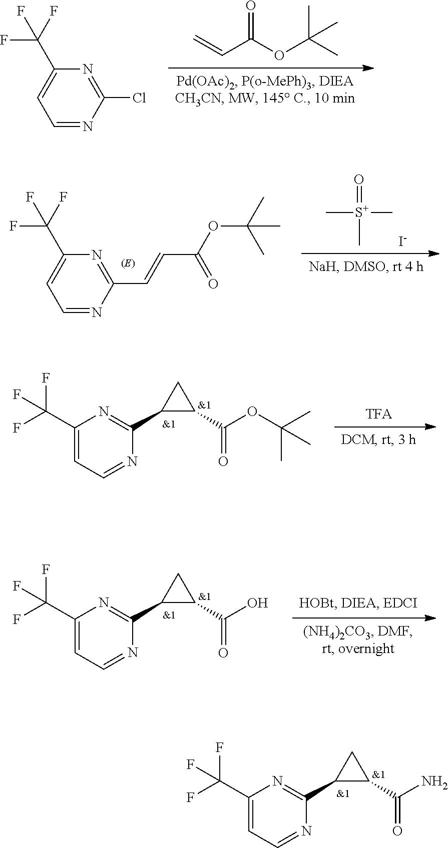

### Synthesis of tert-butyl (E)-3-(4-(trifluoromethyl)pyrimidin-2-yl)acrylate

To a mixture of 2-chloro-4-(trifluoromethyl)pyrimidine (2 g, 11 mmol) and tert-butyl acrylate (4.21 g, 32.9 mmol) in CH₃CN (10 mL) was added Pd(OAc)₂ (490 mg, 2.19 mmol), P(o-MePh)₃ (1 g, 3.29 mmol) and DIEA (5.66 g, 43.83 mmol). The mixture was stirred at 145° C. for 10 min under microwave irradiation. After cooling to 25° C., the reaction was filtered through Celite®. The filtrate was concentrated in vacuo to give the residue, which was purified by silica gel chromatography (eluent: EtOAc/PE=1/100˜20/1) to afford tert-butyl (E)-3-(4-(trifluoromethyl)pyrimidin-2-yl)acrylate (615 mg, 19%) as pale-yellow sticky oil. ESI-MS [M+H]+: 275.1

### Synthesis of rac-tert-butyl (1S*,2S*)-2-(4-(trifluoromethyl)pyrimidin-2-yl)cyclopropane-1-carboxylate

To a mixture of trimethylsulfoxonium iodide (672 mg, 3.05 mmol) in DMSO (5 mL) was added NaH (122 mg, 60% purity in mineral oil, 3.05 mmol) at room temperature. The resulting mixture was stirred for 1 h under N₂ then a solution of tert-butyl (E)-3-(4-(trifluoromethyl)pyrimidin-2-yl)acrylate (465 mg, 1.7 mmol) in DMSO (2 mL) was added. The resulting mixture was stirred at room temperature for 3 h. The reaction was quenched with saturated aqueous NH₄Cl (40 mL) then extracted with EtOAc (5×30 mL). The combined organic layers were dried over Na₂SO₄, filtered and concentrated in vacuo. The residue was purified by silica gel chromatography (EtOAc/PE=1/100˜10/1) to afford rac-tert-butyl (1S*,2S*)-2-(4-(trifluoromethyl)pyrimidin-2-yl)cyclopropane-1-carboxylate (235 mg, 48%) as pale-yellow oil. ESI-MS [M+H]⁺: 289.1

### Synthesis of rac-(1S*,2S*)-2-(4-(trifluoromethyl)pyrimidin-2-yl)cyclopropane-1-carboxylic acid

A mixture of rac-tert-butyl (1S*,2S*)-2-(4-(trifluoromethyl)pyrimidin-2-yl)cyclopropane-1-carboxylate (235 mg, 0.815 mmol) and TFA (2 mL) in DCM (1 mL) was stirred at room temperature for 3 h. The reaction was concentrated in vacuo. The residue was diluted with water (10 mL), then basified with 1 N NaOH solution (3 mL) to pH=9, and washed with EtOAc (2×30 mL). The separated aqueous phase was acidified with 1 N HCl (2 mL) to pH=5, and then extracted with DCM (3×15 mL). The combined organic phase was dried over Na₂SO₄, filtered and concentrated in vacuo to afford rac-(1S*,2S*)-2-(4-(trifluoromethyl)pyrimidin-2-yl)cyclopropane-1-carboxylic acid (140 mg, crude) as white solid, which was used in next step without purification.

ESI-MS [M+H]+: 233.1

### Synthesis of rac-(1S*,2S*)-2-(4-(trifluoromethyl)pyrimidin-2-yl)cyclopropane-1-carboxamide

To a mixture of rac-(1S*,2S*)-2-(4-(trifluoromethyl)pyrimidin-2-yl)cyclopropane-1-carboxylic acid (120 mg, 0.517 mmol), and (NH₄)₂CO₃ (199 mg, 2.067 mmol) in DMF (2 mL) was added HOBt (105 mg, 0.775 mmol), DIPEA (149 mg, 0.775 mmol) and EDCI (333 mg, 2.585 mmol). The mixture was stirred at room temperature overnight. The mixture was diluted with water (30 mL), and then extracted with EtOAc (3×20 mL). The combined organic phase was dried over Na₂SO₄, filtered and concentrated in vacuo. The residue was purified by silica gel chromatography (eluent: EtOAc/PE=1/100-100/1) to afford rac-(1S*,2S*)-2-(4-(trifluoromethyl)pyrimidin-2-yl)cyclopropane-1-carboxamide (80 mg, 67%) as white solid.

ESI-MS [M+H]+: 232.1

### Synthesis of rac-(1S*,2S*)-2-(4-chloropyridin-2-yl)cyclopropane-1-carboxamide

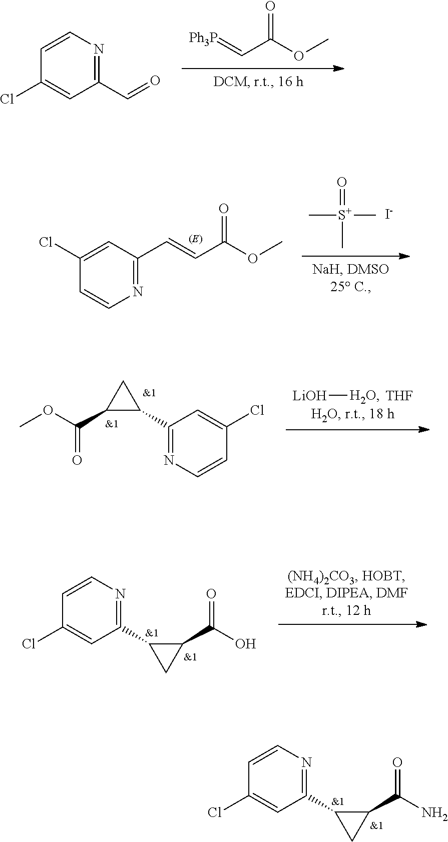

### Synthesis of methyl (E)-3-(4-chloropyridin-2-yl)acrylate

A mixture of 4-chloropicolinaldehyde (2 g, 14.2 mmol) and methyl 2-(triphenyl-15-phosphanylidene)acetate (2.4 g, 14.2 mmol) in DCM (30 mL) was stirred at room temperature for 16 h. The reaction was quenched with water (100 mL) then extracted with DCM (3×50 mL). The combined organic layers were washed with brine (30 mL), dried over Na₂SO₄ and concentrated in vacuo. The residue was purified by silica gel column chromatography (eluent: EtOAc/PE from 0 to 20%) to afford methyl (E)-3-(4-chloropyridin-2-yl)acrylate (2.5 g, 89% yield) as a white solid. ESI-MS [M+H]+: 198.1

### Synthesis of rac-methyl (1S*,2S*)-2-(4-chloropyridin-2-yl)cyclopropane-1-carboxylate

To a mixture of trimethylsulfoxonium iodide (8.4 g, 38.1 mmol) in DMSO (50 mL) was added NaH (1.5 g, 38.1 mmol) and the mixture was stirred at room temperature for 2 h. Methyl (E)-3-(4-chloropyridin-2-yl)acrylate (2.5 g, 12.7 mmol) in DMSO (10 mL) was added and the resulting mixture was stirred at room temperature for 16 h. The reaction was quenched with saturated NH₄Cl solution (100 mL) then extracted with EtOAc (3×100 mL). The combined organic layers were washed with brine (100 mL), dried over Na₂SO₄ and concentrated in vacuo. The residue was purified by silica gel column chromatography (eluent: EtOAc/PE from 0 to 20%) to afford rac-methyl (1S*,2S*)-2-(4-chloropyridin-2-yl)cyclopropane-1-carboxylate (0.7 g, 26% yield) as a yellow oil. ESI-MS [M+H]+: 212.2

### Synthesis of rac-(1S*,2S*)-2-(4-chloropyridin-2-yl)cyclopropane-1-carboxylic acid

A mixture of rac-methyl (1S*,2S*)-2-(4-chloropyridin-2-yl)cyclopropane-1-carboxylate (340 mg, 1.6 mmol) and LiOH—H₂O (135 mg, 3.2 mmol) in THF/H₂O (5 mL/5 mL) was stirred at room temperature for 18 h. The pH of the reaction mixture was adjusted to pH ˜3 with 1N HCl and the mixture was extracted with IPA/CHCl₃ (3/1, 5×30 mL) The combined organic layers were washed with brine (30 mL), dried over Na₂SO₄ and concentrated in vacuo to afford rac-(1S*,2S*)-2-(4-chloropyridin-2-yl)cyclopropane-1-carboxylic acid (300 mg, crude) as an off-white solid, which was used in the next step directly. ESI-MS [M+H]+: 198.2

### Synthesis of rac-(1S*,2S*)-2-(4-chloropyridin-2-yl)cyclopropane-1-carboxamide

A mixture of rac-(1S*,2S*)-2-(4-chloropyridin-2-yl)cyclopropane-1-carboxylic acid (300 mg, 1.52 mmol), (NH₄)₂CO₃ (670 mg, 7.6 mmol), HOBt (410 mg, 3.04 mmol), EDCI (578 mg, 3.04 mmol) and DIEA (588 mg, 4.56 mmol) in DMF (5 mL) was stirred at room temperature for 12 h. The mixture was quenched with water (150 mL) and extracted with EtOAc (5×50 mL). The combined organic layers were washed with brine (50 mL), dried over Na₂SO₄ and concentrated in vacuo. The residue was purified by silica gel column chromatography (EtOAc/PE from 0 to 50%) to rac-(1S*,2S*)-2-(4-chloropyridin-2-yl)cyclopropane-1-carboxamide (220 mg, 73%) as an off-white solid. ESI-MS [M+H]+: 197.2

The mixture was separated using SFC (SFC80, Daicel CHIRALPAK AD-H, 250 mm×20 mm I.D., 5 μm, CO2/MEOH=74/26, 50 g/min, 35° C.) to give two enantiomers: (1S,2S)-2-(4-chloropyridin-2-yl)cyclopropane-1-carboxamide (0.1 g, first eluting isomer, Rt=2.6 min, 99.9% e.e) and (1R,2R)-2-(4-chloropyridin-2-yl)cyclopropane-1-carboxamide (0.1 g, second eluting isomer, Rt=5.5 min, 99.9% e.e)

### Synthesis of rac-(1S*,2S*)-2-(3-cyano-6-methylpyridin-2-yl)cyclopropane-1-carboxamide

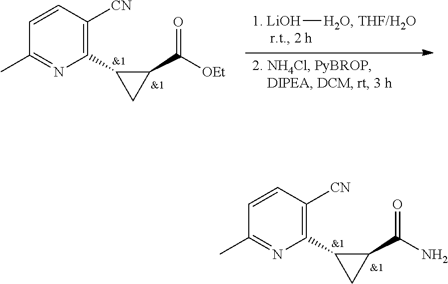

### Synthesis of rac-(1S*,2S*)-2-(3-cyano-6-methylpyridin-2-yl)cyclopropane-1-carboxylic acid

To a mixture of rac-ethyl (1S*,2S*)-2-(3-cyano-6-methylpyridin-2-yl)cyclopropane-1-carboxylate (690 mg, 3 mmol) in THF (10 mL) and water (10 mL) was added LiOH—H₂O (370 mg, 9 mmol). The reaction was stirred at room temperature for 2 h. The pH of the reaction was adjusted to 6 with HCl (1N), and extracted with DCM/MeOH (10/1, 3×30 mL). The combined organic layers were dried over Na₂SO₄, filtered and concentrated in vacuo. The residue was purified by silica gel chromatography (eluent: DCM/MeOH/HCOOH=100/10/1) to give rac-(1S*,2S*)-2-(3-cyano-6-methylpyridin-2-yl)cyclopropane-1-carboxylic acid (330 mg, 54%) as a white solid. ESI-MS [M+H]⁺: 203.1.

### Synthesis of rac-(1S*,2S*)-2-(3-cyano-6-methylpyridin-2-yl)cyclopropane-1-carboxamide

A mixture of rac-(1S*,2S*)-2-(3-cyano-6-methylpyridin-2-yl)cyclopropane-1-carboxylic acid (202 mg, 1 mmol) and NH₄Cl (106 mg, 2 mol), PyBROP (699 mg, 1.5 mmol), DIEA (516 mg, 4 mmol) in DCM (10 mL) was stirred at RT for 2 h. After diluting the mixture with DCM (20 mL), the resulting solution was washed with saturated aqueous NaHCO₃ (30 mL), water (20 mL) then brine (30 mL), dried over Na₂SO₄ and concentrated in vacuo. The residue was purified by preparative TLC (eluent: DCM/MeOH=20/1) to afford rac-(1S*,2S*)-2-(3-cyano-6-methylpyridin-2-yl)cyclopropane-1-carboxamide (160 mg, 80%) as a white solid. ESI-MS [M+H]+: 202.1.

### Synthesis of rac-(1S*,2S*)-2-(5-chloro-2-cyanophenyl)cyclopropane-1-carboxamide

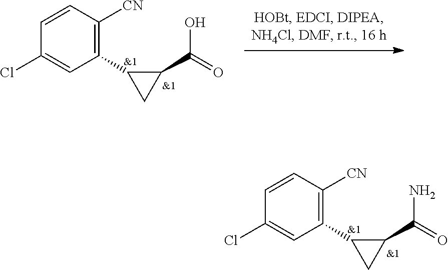

A mixture of rac-(1S*,2S*)-2-(5-chloro-2-cyanophenyl)cyclopropane-1-carboxylic acid (245 mg, 1.1 mmol), NH₄Cl (350 mg, 6.6 mol), HOBt (297 mg, 12.2 mmol), EDCI (420 mg, 2.2 mmol) and DIPEA (851 mg, 6.6 mmol) in DMF (10 mL) was stirred at room temperature under N₂ for 16 h. The reaction was poured onto water (20 ml) and extracted with EtOAc (3×20 ml), The combined organic layers were washed with brine, dried over Na₂SO₄ and concentrated to give the crude, which was purified with preparative TLC (eluent: DCM/MeOH=20/1) to give the rac-(1S*,2S*)-2-(5-chloro-2-cyanophenyl)cyclopropane-1-carboxamide (215 mg, 88%) as white solid. ESI-MS [M+H]⁺: 221.1.

### Synthesis of (1S,2S)-2-(3-chlorophenyl)cyclopropane-1-carboxamide

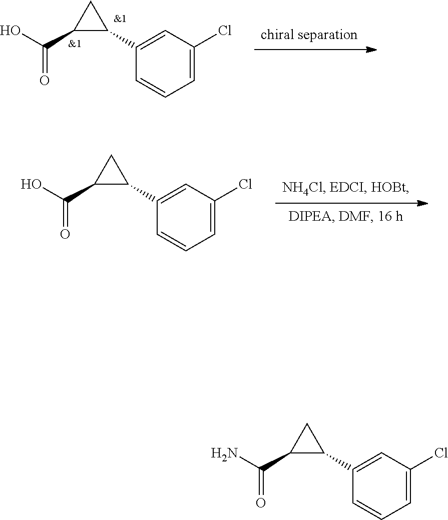 [SMILES: O=C(O)[C@H]1C[C@@H]1c1cccc(Cl)c1 ; "chiral separation" ; O=C(O)[C@H]1C[C@@H]1c1cccc(Cl)c1 ; "DIPEA, DMF, 16 h" ; "NH4Cl, EDCI, HOBt," ; NC(=O)[C@H]1C[C@@H]1c1cccc(Cl)c1]

### Synthesis of (1S,2S)-2-(3-chlorophenyl)cyclopropane-1-carboxylic acid (first eluting isomer)

rac-(1S*,2S*)-2-(3-chlorophenyl)cyclopropane-1-carboxylic acid (10 g) was separated using SFC (SFC80, Daicel CHIRALPAK AD-H 250 mm×20 mm I.D., 5 μm, CO₂/EtOH=86/14, 50 g/min, 35° C.) to give (1S,2S)-2-(3-chlorophenyl)cyclopropane-1-carboxylic acid (4.5 g, first eluting isomer, Rt=3.0 min, 99.9% e.e.) and (1R,2R)-2-(3-chlorophenyl)cyclopropane-1-carboxylic acid (4.3 g, second eluting isomer, Rt=4.0 min, 99.9% e.e.)

### Synthesis of (1S,2S)-2-(3-chlorophenyl)cyclopropane-1-carboxamide

To a solution of (1S,2S)-2-(3-chlorophenyl)cyclopropane-1-carboxylic acid (250 mg, 1.28 mmol) in DMF (4 mL) was added (NH₄)₂CO₃ (247 mg, 2.56 mmol), HOBt (260 mg, 1.92 mmol), EDCI (372 mg, 1.92 mmol) and DIPEA (498 mg, 3.84 mmol). After stirring at room temperature for 12 h, water (20 mL) was added then the reaction was extracted with EtOAc (5×50 mL). The combined organic layers were washed with brine (30 mL), dried over anhydrous Na₂SO₄, and concentrated in vacuo to give the crude, which was purified by silica gel column chromatography (eluent: DCM/MeOH=15/1) to give (1S,2S)-2-(3-chlorophenyl)cyclopropane-1-carboxamide (210 mg, 84%) as a white solid. ESI-MS [M+H]+: 196.1

### Synthesis of rac-methyl (1S*,3S*)-3-(3-chlorophenyl)-2,2-difluorocyclopropane-1-carboxylate

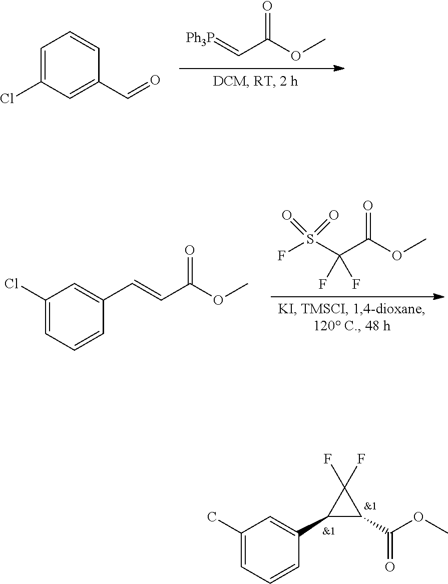

### Synthesis of methyl (E)-3-(3-chlorophenyl)acrylate

A mixture of 3-chlorobenzaldehyde (10 g, 71.4 mmol) and methyl 2-(triphenyl-15-phosphanylidene)acetate (23.9 g, 71.4 mmol) in DCM (200 mL) was stirred at room temperature for 2 h. The reaction was quenched with water (300 mL) then extracted with DCM (3×200 mL). The combined organic layers were washed with brine (200 mL), dried over Na₂SO₄ and concentrated in vacuo. The residue was purified by silica gel column chromatography (eluent: EtOAc/PE from 0 to 10%) to afford methyl (E)-3-(3-chlorophenyl)acrylate (13.5 g, 96% yield) as a white solid.

### Synthesis of rac-methyl (1S*,3S*)-3-(3-chlorophenyl)-2,2-difluorocyclopropane-1-carboxylate

A mixture of methyl (E)-3-(3-chlorophenyl)acrylate (1.0 g, 5.1 mmol), dry KI (2.5 g, 15.3 mmol), TMSCl (1.7 g, 15.3 mmol) and methyl 2,2-difluoro-2-(fluorosulfonyl)acetate (2.9 g, 15.3 mmol) in 1,4-dioxane (10 mL) was stirred at 120° C. for 48h in a sealed tube. After cooled to room temperature, the reaction was quenched with water (40 mL) and extracted with EtOAc (3×50 mL). The combined organic layers were washed with brine (50 mL), dried over Na₂SO₄ and concentrated in vacuo. The residue was purified by silica gel column chromatography (eluent: EtOAc/PE from 0 to 10%) to afford rac-methyl (1S*,3S*)-3-(3-chlorophenyl)-2,2-difluorocyclopropane-1-carboxylate (200 mg, 16% yield) as a yellow oil.

### Synthesis of rac-(1S*,2S*)-2-(4-chloropyrimidin-2-yl)cyclopropane-1-carboxamide

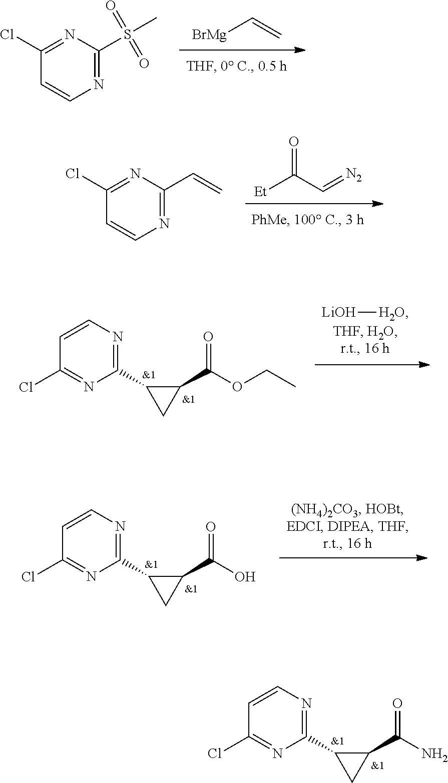

### Synthesis of 4-chloro-2-vinylpyrimidine

To a mixture of 4-chloro-2-(methylsulfonyl)pyrimidine (200 mg, 1.04 mmol) in THF (10 mL) was added vinylmagnesium bromide (2.0M solution in THF, 0.52 mL, 1.04 mmol) under N₂ at 0° C. After stirring at 0° C. for 0.5 h, the reaction was quenched with water (20 mL) and extracted with EtOAc (3×15 mL). The combined organic layers were washed with brine (20 mL), dried over anhydrous Na₂SO₄, and concentrated to give 4-chloro-2-vinylpyrimidine (150 mg, crude) as a yellow oil, which was used in the next step without further purification. ESI-MS [M+H]+: 141.1.

### Synthesis of rac-ethyl (1S*,2S*)-2-(4-chloropyrimidin-2-yl)cyclopropane-1-carboxylate

To a solution of 4-chloro-2-vinylpyrimidine (150 mg, 1.07 mmol) in toluene (5 mL) was added ethyl 2-diazoacetate (360 mg, 3.21 mmol). The resulting reaction mixture was stirred at 100° C. for 3 h under N₂. After cooling to room temperature, the reaction concentrated in vacuo to give the crude, which was purified by silica gel chromatography (eluent: PE/EtOAc=3/1) to give rac-ethyl (1S*,2S*)-2-(4-chloropyrimidin-2-yl)cyclopropane-1-carboxylate (50 mg, 21%) as a yellow solid. ESI-MS [M+H]+: 227.2.

### Synthesis of rac-(1S*,2S*)-2-(4-chloropyrimidin-2-yl)cyclopropane-1-carboxamide

Using a similar procedure to that for 2-(5-chloro-2-nitrophenyl)cyclopropane-1-carboxamide, rac-(1S*,2S*)-2-(4-chloropyrimidin-2-yl)cyclopropane-1-carboxamide (15 mg, 76%) was synthesised from rac-ethyl (1S*,2S*)-2-(4-chloropyrimidin-2-yl)cyclopropane-1-carboxylate (25 mg, 0.11 mmol). ESI-MS [M+H]⁺: 198.2

### Synthesis of rac-(1S*,2S*)-2-(6-chloropyrimidin-4-yl)cyclopropane-1-carboxamide

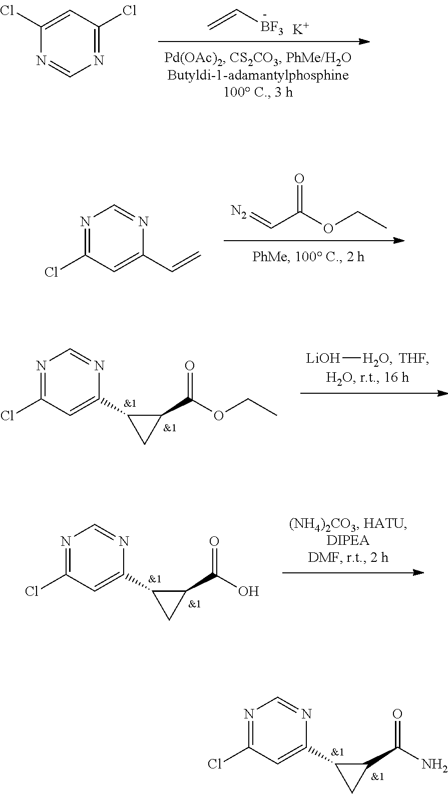

### Synthesis of 4-chloro-6-vinylpyrimidine

A mixture of 4,6-dichloropyrimidine (6 g, 40.2 mmol), potassium ethenyltrifluoroborate (5.4 g, 44.2 mol), Pd(OAc)₂ (900 mg, 4 mmol), butyldi-1-adamantylphosphine (2 g, 6 mmol) and Cs₂CO₃ (26 g, 80.4 mmol) in toluene (120 mL) and water (12 mL) was stirred at 100° C. for 3 h. The reaction mixture was diluted with water (300 mL) then extracted with EtOAc (2×100 mL). The combined organics were washed with brine (100 mL), dried over Na₂SO₄, concentrated in vacuo to give the crude product, which was purified by silica gel column chromatography (eluant: PE:EtOAc=10:1) to give 4-chloro-6-vinylpyrimidine (1.7 g, 30.0% yield) as a colorless oil.

### Synthesis of rac-ethyl (1S*,2S*)-2-(6-chloropyrimidin-4-yl)cyclopropane-1-carboxylate

To a solution of 4-chloro-6-vinylpyrimidine (1.7 g, 12.1 mmol) in toluene (40 mL) was added dropwise a solution of ethyl 2-diazoacetate (4.14 g, 36.3 mmol) in toluene (20 mL). The resulting solution was stirred at 100° C. under N₂ for 2 h, cooled to room temperature and concentrated in vacuo to give the crude product. The crude product was purified by silica gel column chromatography (eluant: PE/EtOAc=10/1) to give ethyl rac-ethyl (1S*,2S*)-2-(6-chloropyrimidin-4-yl)cyclopropane-1-carboxylate (810 mg, 29.5% yield) as colorless oil. ESI-MS [M+H]+: 227.1

### Synthesis of rac-(1S*,2S*)-2-(6-chloropyrimidin-4-yl)cyclopropane-1-carboxylic acid

A mixture of rac-ethyl (1S*,2S*)-2-(6-chloropyrimidin-4-yl)cyclopropane-1-carboxylate (400 mg, 1.66 mmol) and LiOH (120 mg, 5 mmol) in THF (10 mL) and water (2 mL) was stirred at 25° C. for 16 h. After removing THF, the mixture was acidified to pH ˜3 with HCl (1M) then extracted with EtOAc (2×20 mL). The combined organics were washed with brine (50 mL), dried over Na₂SO₄ then concentrated in vacuo to give the crude product. (400 mg, crude). ESI-MS [M+H]+: 199.1

### Synthesis of rac-(1S*,2S*)-2-(6-chloropyrimidin-4-yl)cyclopropane-1-carboxamide

A mixture of rac-(1S*,2S*)-2-(6-chloropyrimidin-4-yl)cyclopropane-1-carboxylic acid (400 mg, 2 mmol), HATU (384 mg, 4 mmol) and DIPEA (755 mg, 6 mmol) in DMF (20 mL) was stirred at 25° C. for 10 mins. (NH₄)₂CO₃ (384 mg, 4 mmol) was added then the mixture was stirred at 25° C. for 2 h. The mixture was diluted with water (200 mL) then extracted with EtOAc (3×50 mL). The combined organics were washed with brine (100 mL), dried over Na₂SO₄ then concentrated in vacuo to give the crude product. The crude product was trituated with PE:DCM=1:1 to give the rac-(1S*,2S*)-2-(6-chloropyrimidin-4-yl)cyclopropane-1-carboxamide as an off-white solid. (300 mg, 75% yield). ESI-MS [M+H]+: 198.1

### Synthesis of rac-methyl (1S*,2S*)-2-(3-chlorophenyl)-3-fluorocyclopropane-1-carboxylate

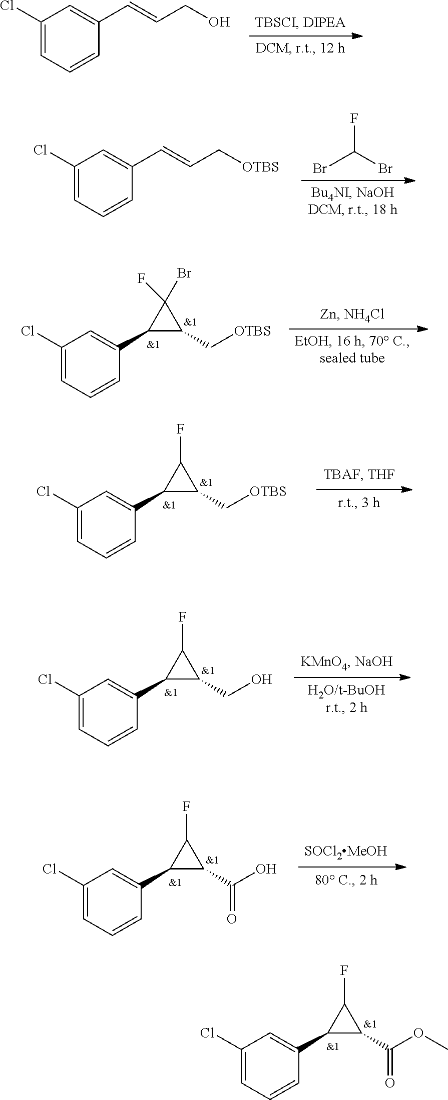

### Synthesis of (E)-tert-butyl((3-(3-chlorophenyl)allyl)oxy)dimethylsilane

To a solution of (E)-3-(3-chlorophenyl)prop-2-en-1-ol (300 mg, 1.78 mmol) and DIPEA (807 mg, 6.23 mmol) in DCM (10 mL) was added TBSCl (538 mg, 3.56 mmol). The resulting mixture was stirred at room temperature for 12 h. Water (30 mL) was added to the reaction, extracted with DCM (3×30 mL). The combined organic layers were washed with brine (30 mL), dried over anhydrous Na₂SO₄, and concentrated in vacuo to give the crude, which was purified by silica gel column chromatography (eluent: PE/EtOAc=1/1) to give (E)-tert-butyl((3-(3-chlorophenyl)allyl)oxy)dimethylsilane (300 mg, 59%) as a yellow oil. ESI-MS [M+H]+: 283.1.

### Synthesis of rac-(((1S*,3S*)-2-bromo-3-(3-chlorophenyl)-2-fluorocyclopropyl)methoxy)(tert-butyl)dimethylsilane

A mixture of (E)-tert-butyl((3-(3-chlorophenyl)allyl)oxy)dimethylsilane (300 mg, 1.06 mmol), dibromofluoromethane (305 mg, 1.59 mmol) and Bu₄NI (39 mg, 0.106 mmol) in DCM (1 mL)/50% NaOH aqueous (1 mL). The mixture was degassed with N₂ for 1 min, then stirred at 25° C. in a sealed tube for 18 h. The reaction mixture was quenched with water (10 mL) and extracted with EtOAc (3×30 mL). The combined organic layers were washed with brine (20 mL), dried over anhydrous Na₂SO₄, and concentrated in vacuo to give the crude product, which was purified by silica gel column chromatography (eluent: PE/EtOAc=100/0) to give rac-(((1S*,3S*)-2-bromo-3-(3-chlorophenyl)-2-fluorocyclopropyl)methoxy)(tert-butyl)dimethylsilane (160 mg, 38.3%) as a yellow oil. ESI-MS [M+H]+: 393.1.

### Synthesis of rac-tert-butyl(((1S*,2S*)-2-(3-chlorophenyl)-3-fluorocyclopropyl)methoxy)dimethylsilane

A mixture of rac-(((1S*,3S*)-2-bromo-3-(3-chlorophenyl)-2-fluorocyclopropyl)methoxy)(tert-butyl)dimethylsilane (160 mg, 0.4 mmol), Zn (240 mg, 3.6 mmol) and NH₄Cl (196 mg, 3.6 mmol) in EtOH (5 mL) was degassed with N₂ for 1 min then stirred in a sealed tube at 70° C. for 16 h. After cooling to room temperature, the reaction mixture was filtered through Celite® and the filter cake was washed with DCM/MeOH (10/1, 40 mL). The filtrate was concentrated to give the crude, which was purified by silica gel column chromatography (eluent: DCM/MeOH=50/1˜20/1) to give rac-tert-butyl(((1S*,2S*)-2-(3-chlorophenyl)-3-fluorocyclopropyl)methoxy)dimethylsilane (120 mg, 93.7%) as a yellow oil. ESI-MS [M+H]+: 315.1.

### Synthesis of rac-((1S*,2S*)-2-(3-chlorophenyl)-3-fluorocyclopropyl)methanol

To a mixture of rac-tert-butyl(((1S*,2S*)-2-(3-chlorophenyl)-3-fluorocyclopropyl)methoxy)dimethylsilane (120 mg, 0.38 mmol) in THF (10 mL) was added TBAF (250 mg, 0.95 mmol). The reaction mixture was stirred at 25° C. for 3 h under N₂. Water (10 mL) was added, and the reaction mixture was extracted with EtOAc (3×30 mL). The combined organic layers were washed with brine (30 mL), dried over anhydrous Na₂SO₄, and concentrated in vacuo to give the crude product, which was purified by silica gel column chromatography (eluent: PE/EtOAc=1/1) to give rac-((1S*,2S*)-2-(3-chlorophenyl)-3-fluorocyclopropyl)methanol (90 mg, 90%) as yellow oil. ESI-MS [M+H]+: 201.1.

### Synthesis of rac-(1S*,2S*)-2-(3-chlorophenyl)-3-fluorocyclopropane-1-carboxylic acid

To a mixture of rac-((1S*,2S*)-2-(3-chlorophenyl)-3-fluorocyclopropyl)methanol (90 mg, 0.45 mmol) in t-BuOH (2 mL) was added a mixture of NaOH (144 mg, 3.6 mmol) and KMnO₄ (213 mg, 1.35 mmol) in water (1.5 ml). After stirring at 25° C. for 2 h under N₂, Water (20 mL) was added, the mixture was extracted with DCM (3×30 mL). The combined organic layers were washed with brine (10 mL), dried over anhydrous Na₂SO₄, and concentrated in vacuo to give the crude product, which was purified by silica gel column chromatography (eluent: DCM/MeOH=15/1) to give rac-(1S*,2S*)-2-(3-chlorophenyl)-3-fluorocyclopropane-1-carboxylic acid (70 mg, 72.6%) as a yellow oil. ESI-MS [M+H]+: 215.1.

### Synthesis of rac-methyl (1S*,2S*)-2-(3-chlorophenyl)-3-fluorocyclopropane-1-carboxylate

To a mixture of rac-(1S*,2S*)-2-(3-chlorophenyl)-3-fluorocyclopropane-1-carboxylic acid (70 mg, 0.32 mmol) in MeOH (4 mL) was added SOCl₂ (0.4 ml). The reaction mixture was stirred at 80° C. for 2 h under N₂, cooled to room temperature then concentrated in vacuo to give the crude product, which was purified by preparative TLC (eluent: DCM/MeOH=20/1) to give rac-methyl (1S*,2S*)-2-(3-chlorophenyl)-3-fluorocyclopropane-1-carboxylate (40 mg, 54%) as a yellow solid. ESI-MS [M+H]+: 229.1.

### Synthesis of rac-(1S*,2S*)-2-(5-chloropyridazin-3-yl)cyclopropane-1-carboxamide

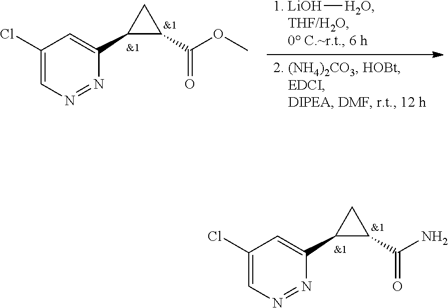

Using a similar procedure to that for 2-(5-chloro-2-nitrophenyl)cyclopropane-1-carboxamide, rac-(1S*,2S*)-2-(5-chloropyridazin-3-yl)cyclopropane-1-carboxamide (5 mg, 12%) was synthesised from rac-ethyl (1S*,2S*)-2-(5-chloropyridazin-3-yl)cyclopropane-1-carboxylate (210 mg, 0.93 mmol). ESI-MS [M+H]+: 198.2.

### Synthesis of rac-(1S*,2S*)-2-(5-chloro-3-cyanothiophen-2-yl)cyclopropane-1-carboxylic acid

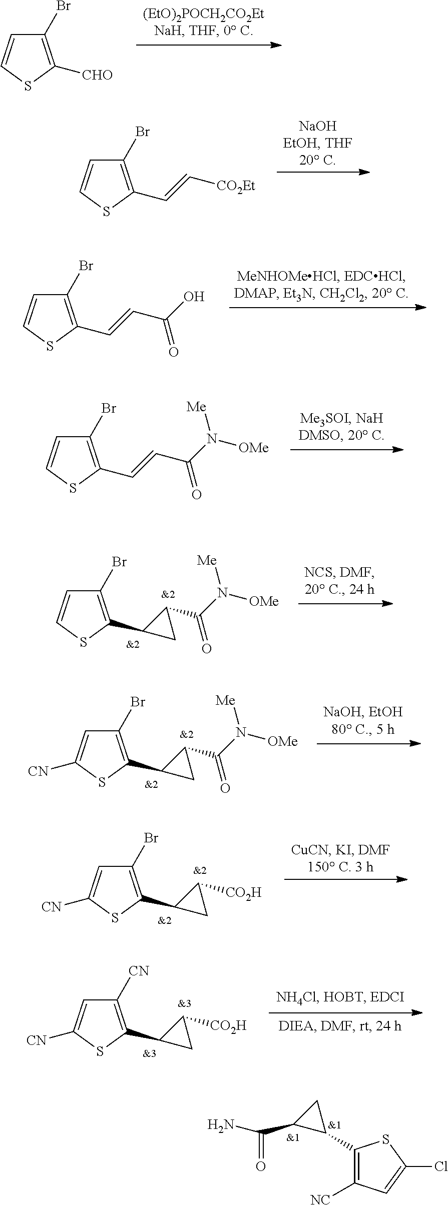

### Synthesis of ethyl (E)-3-(3-bromothiophen-2-yl)acrylate

Sodium hydride (60% oil dispersion, 2.76 g, 69.0 mmol) was suspended in anhydrous THF (500 mL) under nitrogen at 0° C. Triethyl phosphonoacetate (14.7 g, 65.5 mmol) was added dropwise over 5 min and the mixture was stirred for 30 min. 3-Bromo-2-thiophenecarboxaldehyde (10.0 g, 52.4 mmol) was added dropwise over 5 min. The mixture was allowed to warm to room temperature and stirred for a further 18 h. The reaction mixture was poured into saturated aqueous ammonium chloride (100 mL) and extracted with methyl tert-butyl ether (3×100 mL). The combined organic extracts were washed with brine (2×100 mL), dried (MgSO₄), filtered and concentrated in vacuo to give a pale yellow solid. The solid was purified by column chromatography (200 g silica gel, eluting with 0-25% ethyl acetate in heptane) to give ethyl (E)-3-(3-bromothiophen-2-yl)acrylate (10.9 g, 79%), as a colourless solid. ESI-MS [M+H]⁺ 263, ¹H NMR (400 MHz, CDCl₃) δ 7.83 (dd, J 15.7, 1.2 Hz, 1H), 7.33 (d, J 5.4 Hz, 1H), 7.03 (d, J 5.4 Hz, 1H), 6.30 (d, J 15.7 Hz, 1H), 4.27 (q, J 7.3 Hz, 2H), 1.33 (t, J 7.3 Hz, 3H).

### Synthesis of (E)-3-(3-bromothiophen-2-yl)acrylic acid

Aqueous sodium hydroxide (2 M, 85.7 mL, 171 mmol) was added to a solution of ethyl (E)-3-(3-bromothiophen-2-yl)acrylate (10.9 g, 41.6 mmol) in a mixture of THF (120 mL) and ethanol (60 mL), and the mixture was stirred for 18 h at room temperature. The mixture was acidified to pH 2 using hydrochloric acid (2M, 100 mL) and extracted with ethyl acetate (3×200 mL). The combined extracts were washed with water (2×100 mL), brine (2×100 mL), dried (MgSO₄), filtered and concentrated in vacuo to give (E)-3-(3-bromothiophen-2-yl)acrylic acid (9.26 g, 96%), as a colourless solid. ESI-MS [M−H]⁻ 231/233, ¹H NMR (400 MHz, CD₃SOCD₃) δ 12.63 (s, 1H), 7.85 (d, J 5.4 Hz, 1H), 7.63 (d, J 16.3 Hz, 1H), 7.25 (d, J 5.4 Hz, 1H), 6.30 (d, J 16.3 Hz, 1H).

### Synthesis of (E)-3-(3-bromothiophen-2-yl)-N-methoxy-N-methylacrylamide

A solution of (E)-3-(3-bromothiophen-2-yl)acrylic acid (9.26 g, 39.7 mmol) and N-ethyl-N′N′-diemthylaminopropylcarbodiimide hydrochloride (10.66 g, 55.63 mmol) in DCM (150 mL) under nitrogen was cooled to 0° C. N,N-Dimethylaminopyridine (5.29 g, 43.3 mmol), N,O-dimethylhydroxylamine hydrochloride (4.88 g, 50.1 mmol) and triethylamine (11.6 mL, 83.4 mmol) were added and the reaction mixture was stirred for 18 h at room temperature. Further N-ethyl-N′N′-diemthylaminopropylcarbodiimide hydrochloride (2.13 g, 11.1 mmol), N,O-dimethylhydroxylamine hydrochloride (0.976 g, 10.0 mmol) and triethylamine (2.3 mL, 17 mmol) were added. The reaction mixture was stirred for another 18 h at room temperature. The reaction mixture was diluted with ethyl acetate (500 mL) and the solution was washed with water (2×100 mL), brine (2×100 mL), dried (MgSO₄), filtered and concentrated in vacuo to give a yellow oil. Purification by flash chromatography (Silica gel, eluting with 0-80% ethyl acetate in heptane) gave (E)-3-(3-bromothiophen-2-yl)-N-methoxy-N-methylacrylamide (8.39 g, 76%), as a colourless solid. ESI-MS [M+H]⁺ 276/278¹H NMR (400 MHz, CDCl₃) δ 7.88 (d, J 15.7 Hz, 1H), 7.30 (d, J 5.4 Hz, 1H), 7.02 (d, J 5.4 Hz, 1H), 6.90 (d, J 15.7 Hz, 1H), 3.76 (s, 3H), 3.30 (s, 3H).

### Synthesis of (rac)-(1S*,2S*)-2-(3-bromothiophen-2-yl)-N-methoxy-N-methylcyclopropane-1-carboxamide

Sodium hydride (60% oil dispersion, 2.57 g, 107 mmol) was washed with heptane (2×50 mL) and suspended in anhydrous DMSO (30 mL) under nitrogen at room temperature. A solution of trimethylsulfoxonium iodide (21.8 g, 99.0 mmol) in anhydrous DMSO (90 mL) was added dropwise over 10 min to give a cloudy suspension. The mixture was stirred at room temperature for 1 h, during which time it became a pale-yellow solution. Then a solution of (E)-3-(3-bromothiophen-2-yl)-N-methoxy-N-methylacrylamide (8.39 g, 30.4 mmol) in anhydrous DMSO (30 mL) was added dropwise over 5 min. The mixture was stirred at room temperature overnight, diluted with ethyl acetate (250 mL) and the solution was washed with water (3×70 mL), brine (3×80 mL), dried (MgSO₄), filtered and concentrated in vacuo to give a yellow oil. Purification by column chromatography (silica gel, eluting with 0-80% ethyl acetate in heptane) gave (rac)-(1S*,2S*)-2-(3-bromothiophen-2-yl)-N-methoxy-N-methylcyclopropane-1-carboxamide (7.46 g, 85%), as a colourless oil. ESI-MS [M+H]⁺ 290/292, ¹H NMR (400 MHz, CDCl₃) δ 7.06 (d, J 5.2 Hz, 1H), 6.91 (d, J 5.2 Hz, 1H), 3.75 (s, 3H), 3.25 (s, 3H), 2.65 (ddd, J 9.6, 6.0, 4.0 Hz, 1H), 2.45 (br s, 1H), 1.70 (ddd, J 9.6, 5.4, 4.0 Hz, 1H), 1.30 (ddd, J 8.5, 6.0, 4.0 Hz, 1H).

### Synthesis of (rac)-(1S*,2S*)-2-(3-bromo-5-chlorothiophen-2-yl)-N-methoxy-N-methylcyclopropane-1-carboxamide

N-Chlorosuccinimide (2.02 g, 15.2 mmol) was added to a solution of (rac)-(1S*,2S*)-2-(3-bromothiophen-2-yl)-N-methoxy-N-methylcyclopropane-1-carboxamide (4.0 g, 13.78 mmol) in DMF (103 mL) at room temperature under nitrogen. The mixture was stirred for 24 h, diluted with ethyl acetate and washed with aqueous sodium thiosulfate (10%, 3×40 mL). The organic solution was washed with water (4×50 mL), brine (4×50 mL), dried (MgSO₄), filtered and concentrated in vacuo to give a yellow oil. Analysis by LCMS showed the reaction was incomplete. Consequently, the oil was dissolved in DMF (20 mL) at room temperature and more N-chlorosuccinimide (707 mg, 5.28 mmol) was added. The reaction mixture was stirred for 6 h at room temperature. The reaction mixture was diluted with ethyl acetate (200 mL) and washed with aqueous sodium thiosulfate (10%, 3×60 mL), water (4×80 mL), brine (4×80 mL), dried (MgSO₄), filtered and concentrated in vacuo to give a yellow oil. Purification by column chromatography (silica gel, eluting with 0-80% ethyl acetate in heptane) gave (rac)-(1S*,2S*)-2-(3-bromo-5-chlorothiophen-2-yl)-N-methoxy-N-methylcyclopropane-1-carboxamide (3.80 g, 85%), as a colourless solid. ESI-MS [M+H]⁺ 326/328, ¹H NMR (400 MHz, CDCl₃) δ 6.75 (s, 1H), 3.77 (s, 3H), 3.25 (s, 3H), 2.60-2.55 (m, 1H), 2.42 (m, 1H), 1.70-1.65 (m, 1H), 1.26-1.22 (m, 1H).

### Synthesis of (rac)-(1S*,2S*)-2-(3-bromo-5-chlorothiophen-2-yl)-cyclopropane-1-carboxylic acid

A solution of sodium hydroxide (2 M in water, 11.72 mL, 23.45 mmol) was added to a solution of (rac)-(1S*,2S*)-2-(3-bromo-5-chlorothiophen-2-yl)-N-methoxy-N-methylcyclopropane-1-carboxamide (3.8 g, 11.72 mmol) in EtOH (46.0 mL) at room temperature under nitrogen. The mixture was stirred at 80° C. for 5 h. The mixture was acidified to pH 2 using hydrochloric acid (2M, 15 mL) and extracted with ethyl acetate (2×100 mL). The extracts were washed with water (2×60 mL), brine (2×60 mL), dried (MgSO₄), filtered and concentrated in vacuo. The residue was recrystallised from ethyl acetate in heptane to give (rac)-(1S*,2S*)-2-(3-bromo-5-chlorothiophen-2-yl)-cyclopropane-1-carboxylic acid (3.18 g, 96%), as a colourless solid. ESI-MS [M−H]⁻ 281/283, ¹H NMR (400 MHz, CD₃SOCD₃) δ 12.52 (s, 1H), 7.15 (s, 1H), 2.38 (ddd, J 9.7, 5.4, 3.6 Hz, 1H), 1.80-1.76 (m, 1H), 1.48-1.43 (m, 1H), 1.29-1.24 (m, 1H).

### Synthesis of (rac)-(1S*,2S*)-2-(5-chloro-3-cyanothiophen-2-yl)-cyclopropane-1-carboxylic acid

A mixture of (rac)-(1S*,2S*)-2-(3-bromo-5-chlorothiophen-2-yl)-cyclopropane-1-carboxylic acid (1.00 g, 3.55 mmol), copper cyanide (956 mg, 10.6 mmol) and potassium iodide (59 mg, 0.35 mmol) in anhydrous DMF (4 mL) was heated at 150° C. under nitrogen for 3 h. After being cooled to room temperature, the mixture was diluted with ethyl acetate (100 mL) and filtered through short pad of Dicalite (2 g). The filtrate was acidified with hydrochloric acid (2M, 6 mL) to give pH 2. The aqueous layer was separated and extracted with ethyl acetate (2×100 mL). the combined organic solutions were washed with brine (2×50 mL), dried (MgSO₄), filtered and concentrated under reduced pressure. The residue was purified by reverse phase chromatography, eluting with a gradient of acetonitrile and 0.1% aqueous formic acid=10:90 to 90:10 over 10 column volumes. Fractions containing product were pooled, concentrated in vacuo and freeze-dried to give (rac)-(1S*,2S*)-2-(5-chloro-3-cyanothiophen-2-yl)-cyclopropane-1-carboxylic acid (545 mg, 69%), as a colourless solid. ESI-MS [M−H]⁻ 226/228, ¹H NMR (400 MHz, CD₃SOCD₃) δ 12.67 (s, 1H), 7.47 (s, 1H), 2.65 (ddd, J 10, 6, 4.4 Hz, 1H), 2.00 (ddd, J 9.2, 4.8, 4.4 Hz, 1H), 1.56 (dt, J 10, 4.8 Hz, 1H), 1.41 (ddd, J 9.2, 6, 4.8 Hz, 1H).

### Synthesis of rac-(1S*,2S*)-2-(5-chloro-3-cyanothiophen-2-yl)cyclopropane-1-carboxamide

A mixture of rac-(1S*,2S*)-2-(5-chloro-3-cyanothiophen-2-yl)cyclopropane-1-carboxylic acid (270 mg, 1.19 mmol), NH₄Cl (378 mg, 7.14 mol), HOBt (321 mg, 2.38 mmol), EDCI (455 mg, 2.38 mmol) and DIPEA (921 mg, 7.14 mmol) in DMF (12 mL) was stirred at room temperature under N₂ for 24 h. The reaction was poured into water (20 ml) then extracted with EtOAc (3×30 ml). The combined organic layers were washed with brine (30 ml), dried with Na₂SO₄ then concentrated in vacuo to give the crude, which was purified by silica gel chromatography (DCM/MeOH=30:1) to rac-(1S*,2S*)-2-(5-chloro-3-cyanothiophen-2-yl)cyclopropane-1-carboxamide (210 mg, 78%) as a yellow solid. ESI-MS [M+H]+: 227.1.

### Syntheses of methyl rac-(1R*,2S*)-5′-chloro-1′-methyl-2′-oxospiro[cyclopropane-1,3′-indoline]-2-carboxylate

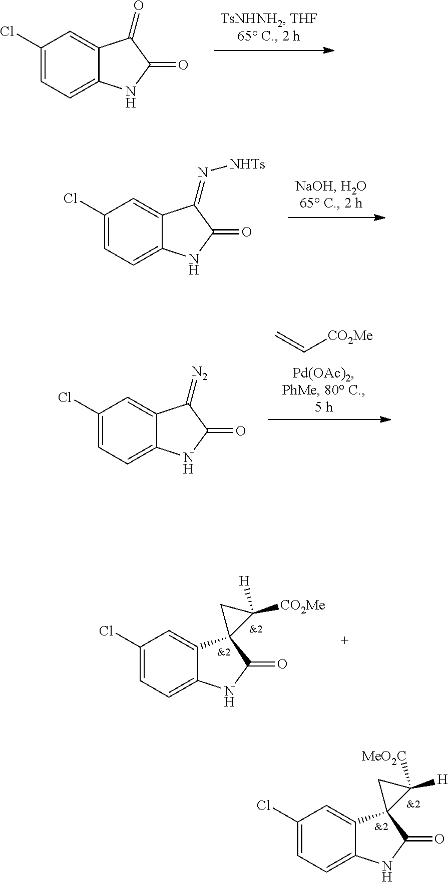

### Synthesis of (Z)—N′-(5-chloro-2-oxoindolin-3-ylidene)-4-methylbenzenesulfonohydrazide

5-Chloroisatin (20.0 g, 110 mmol) was suspended in THF (400 mL). Tosylhydrazine (21.5 g, 115 mmol) was added, and the suspension was heated at reflux for 2 h. The reaction mixture was allowed to cool to room temperature, and the product was collected by filtration, washed with cold methanol and dried in vacuo. (Z)—N′-(5-Chloro-2-oxoindolin-3-ylidene)-4-methylbenzenesulfonohydrazide (34.6 g, 90%) was obtained as a yellow solid. ESI-MS [M+H]⁺ 350. ¹H-NMR (400 MHz, DMSO): δ 12.46 (s, 1H), 11.30 (s, 1H), 7.88 (d, J 7.9 Hz, 2H), 7.45 (d, J 7.9 Hz, 2H and d, J 2.4 Hz, 1H), 7.39 (dd, J 8.8, 2.4 Hz, 1H), 6.91 (d, J 8.8 Hz, 1H), 2.39 (s, 3H).

### Synthesis of 5-chloro-3-diazoindolin-2-one

(Z)—N′-(5-Chloro-2-oxoindolin-3-ylidene)-4-methylbenzenesulfonohydrazide (12.0 g, 38.1 mmol) was treated with a solution of sodium hydroxide (3.04 g, 76.1 mmol) in water (375 mL). The reaction mixture was stirred at 65° C. for 2 h, and then allowed to cool to room temperature. The reaction mixture was neutralized (from pH 11 to pH7) by the gradual addition of solid carbon dioxide with cooling in an ice-water bath. The mixture was filtered and the solid dried in vacuo to give 5-chloro-3-diazoindolin-2-one (7.01 g, 99%), as an orange solid. ¹H NMR (400 MHz, DMSO): δ 10.78 (s, 1H), 7.58 (d, J 1.8 Hz, 1H), 7.12 (dd, J 8.5, 1.8 Hz, 1H), 6.89 (d, J 8.5 Hz, 1H).

### Methyl rac-(1R*, 2S*)-5′-chloro-2′-oxospiro[cyclopropane-1,3′-indoline]-2-carboxylate and methyl rac-(1R*, 2R*)-5′-chloro-2′-oxospiro[cyclopropane-1,3′-indoline]-2-carboxylate

A mixture of 5-chloro-3-diazoindolin-2-one (3.40 g, 17.6 mmol), methyl acrylate (3.40 mL, 37.5 mmol) and palladium acetate (478 mg, 2.13 mmol) in anhydrous toluene (100 mL) was heated under nitrogen at 80° C. for 5 h. The reaction mixture was concentrated under reduced pressure. The residue was dissolved in ethyl acetate (40 mL), adsorbed onto silica gel (35 g), and purified by flash chromatography (Biotage Isolera, 330 g silica cartridge, eluting with 25% ethyl acetate in heptane for 2 column volumes, then 25-75% ethyl acetate in heptane over 12 column volumes). Fractions were collected together to give a mixture of the impure desired product isomers. This material was combined with another batch, prepared on the same scale in the same manner already described, to give a red solid (2.128 g). Two other impure batches prepared previously (0.150 g and 0.317 g respectively) were also added. The combined crude material (2.595 g) was adsorbed onto silica gel (10 g) with acetone (40 mL) and re-purified by flash chromatography (eluting with 10% methyl tert-butyl ether in toluene for 1 column volume, followed by 10-33% methyl tert-butyl ether in toluene over 8 column volumes and 33-50% methyl tert-butyl ether in toluene over 3 column volumes).

First eluted was the (1R*,2R*)-isomer (structure assigned by NMR)

ESI-MS [M+H]⁺: 252. ¹H NMR (400 MHz, CDCl₃) δ 8.44 (s, 1H), 7.37 (d, J 2.4 Hz, 1H), 7.21 (dd, J 8.5, 2.4 Hz, 1H), 6.87 (d, J 8.5 Hz, 1H), 3.72 (s, 3H), 2.73 (dd, J 8.5, 7.5 Hz, 1H), 2.15 (dd, J 7.5, 4.5 Hz, 1H), 2.06 (dd, J 8.5, 4.5 Hz, 1H).

Second eluted was methyl (1R*,2S*)-isomer (structure assigned by NMR) (1.230 g, 12%), as an orange solid.

ESI-MS [M+H]⁺: 252, ¹H NMR (400 MHz, CDCl₃) δ 8.35 (s, 1H), 7.20 (dd, J 8.5, 1.8 Hz, 1H), 6.87 (d, J 8.5 Hz, 1H), 6.81 (d, J 1.8 Hz, 1H), 3.74 (s, 3H), 2.67 (t, J 8.5 Hz, 1H), 2.41 (dd, J 8.5, 5 Hz, 1H), 1.84 (dd, J 8.5, 5 Hz, 1H).

### Synthesis of rac-(1R*,2S*)-5′-chloro-1′-methyl-2′-oxospiro[cyclopropane-1,3′-indoline]-2-carboxamide

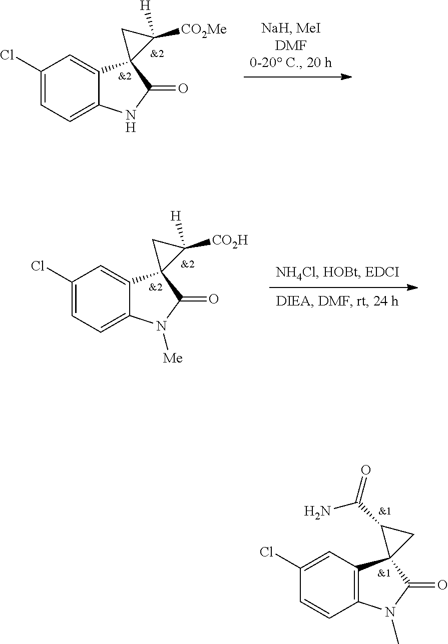 [SMILES: COC(=O)[C@@H]1C[C@]12C(=O)Nc1ccc(Cl)cc12 ; "NaH, MeIDMF0-20° C., 20 h" ; CN1C(=O)[C@]2(C[C@H]2C(=O)O)c2cc(Cl)ccc21 ; "DIEA, DMF, rt, 24 h" ; "NH4Cl, HOBt, EDCI" ; CN1C(=O)[C@]2(C[C@H]2C(N)=O)c2cc(Cl)ccc21]

A solution of methyl rac-(1R*,2S*)-5′-chloro-2′-oxospiro[cyclopropane-1,3′-indoline]-2-carboxylate (630 mg, 2.50 mmol) in anhydrous THF (100 mL) was added to a stirred suspension of sodium hydride (120 mg, 60% oil dispersion, 5.00 mmol) in anhydrous THF (25 mL) under nitrogen at 0° C. The mixture was stirred at room temperature for 1.5 h then re-cooled to 0° C. Iodomethane (0.32 mL, 5.00 mmol) was added and the mixture was stirred at 0° C. for 2 h and then at room temperature for 18 h. The mixture was treated with aqueous citric acid (10% w/v, 18 mL) and the aqueous layer was extracted with ethyl acetate (3×50 mL) and dichloromethane (30 mL). The combined organic solutions were washed with brine (2×50 mL), dried (MgSO₄) and concentrated under reduced pressure. The residue was purified by reversed-phase column chromatography, eluting with a gradient of 5-70% acetonitrile in water containing 0.1% formic acid over 14 column volumes. Fractions containing the product were pooled and concentrated in vacuo to give (1R*,2S*)-5′-chloro-1′-methyl-2′-oxospiro[cyclopropane-1,3′-indoline]-2-carboxylic acid (304 mg, 48%), as a colourless solid. ESI-MS [M+H]⁺ 252, ¹H NMR (400 MHz, CDCl₃) δ 7.34 (dd, J 8, 2 Hz, 1H), 6.95 (d, J 8 Hz, 1H), 6.86 (d, J 2 Hz, 1H), 3.38 (s, 3H), 2.89 (dd, J 9.7, 8.5 Hz, 1H), 2.32 (dd, J 8.5, 5.3 Hz, 1H), 2.16 (dd, J 9.7, 5.3 Hz, 1H).

### Synthesis of rac-(1R*,2S*)-5′-chloro-1′-methyl-2′-oxospiro[cyclopropane-1,3′-indoline]-2-carboxamide

A mixture of rac-(1R*,2S*)-5′-chloro-1′-methyl-2′-oxospiro[cyclopropane-1,3′-indoline]-2-carboxylic acid (300 mg, 1.19 mmol), NH₄Cl (378 mg, 7.14 mol), HOBt (321 mg, 2.38 mmol), EDCI (455 mg, 2.38 mmol) and DIPEA (921 mg, 7.14 mmol) in DMF (12 mL) was stirred at room temperature under N₂ for 24 h. The reaction was poured into water (20 ml) and extracted with EtOAc (30 ml×4). The combined organic layers were washed with brine (30 ml), dried over Na₂SO₄ then concentrated in vacuo to give the crude, which was purified by silica gel chromatography (eluant: DCM/MeOH=30:1) to give the rac-(1R*,2S*)-5′-chloro-1′-methyl-2′-oxospiro[cyclopropane-1,3′-indoline]-2-carboxamide (250 mg, 84%) as a yellow solid. ESI-MS [M+H]+: 251.1.

### Synthesis of rac-(1R*,2S*,3R*)-2-(3-chlorophenyl)-3-(hydroxymethyl)cyclopropane-1-carboxamide chlorophenyl)acrylate

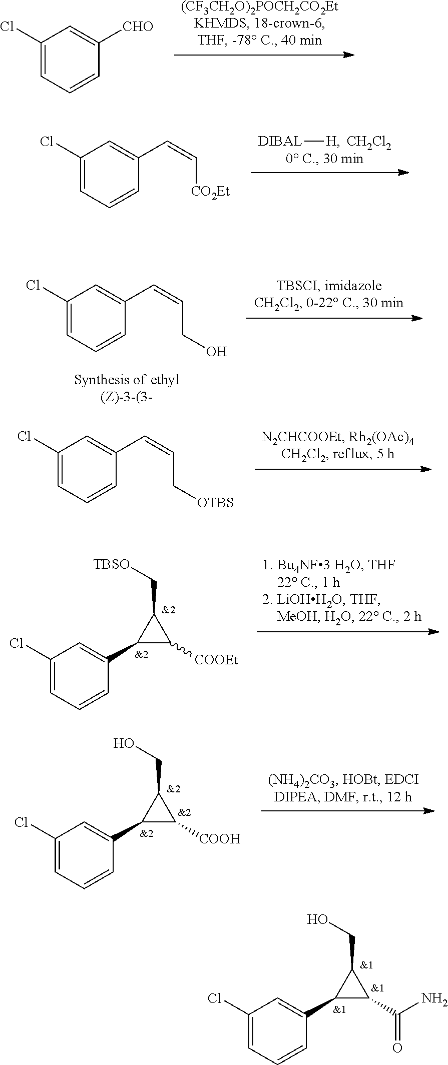

To a −78° C. solution of ethyl bis(2,2,2-trifluoroethyl)phosphonoacetate (2.51 g, 7.56 mmol) and 18-crown-6 (9.51 g, 36.0 mmol) in anhydrous THF (100 mL) was added potassium bis(trimethylsilyl)amide (11% w/v in toluene, 15.6 mL, 7.56 mmol) dropwise over 5 min. After being stirred for 10 min, a solution of 3-chlorobenzaldehyde (816 μL, 7.20 mmol) in anhydrous THF (5 mL) was added over ca. 5 min. After being stirred for 40 min the reaction was quenched by the addition of saturated aqueous ammonium chloride (50 mL) and the resulting suspension was allowed to warm to room temperature. Most of the solvent was removed under reduced pressure and the biphasic mixture was diluted with ethyl acetate (100 mL). The layers were separated, and the organic layer was washed with water 3 times, and brine. The solution was dried (MgSO₄), filtered and concentrated under reduced pressure to give a light-yellow oil. Purification on silica gel (40 g), eluting with a gradient of heptane:ethyl acetate=100:0 to 97:3 afforded ethyl (Z)-3-(3-chlorophenyl)acrylate (1.43 g, 86%), as a colourless liquid. ¹H NMR (400 MHz, CDCl₃, ppm) δ 7.58-7.56 (m, 1H), 7.44-7.40 (m, 1H), 7.32-7.27 (m, 2H), 6.88 (d, J 13 Hz, 1H), 5.99 (d, J 13 Hz, 1H), 4.18 (q, J 7 Hz, 2H), 1.25 (t, J 7 Hz, 3H).

### Synthesis of (Z)-3-(3-chlorophenyl)prop-2-en-1-ol

A solution of ethyl (Z)-3-(3-chlorophenyl)acrylate (1.43 g, 6.80 mmol) in anhydrous DCM (47 mL) under nitrogen was cooled in an ice bath and a solution of diisobutylaluminium hydride (25% in toluene, 10.0 mL, 15.0 mmol) was added dropwise over ca. 10 min. After addition was complete, the mixture was stirred for another 20 min before being poured into 5% aqueous sodium potassium tartrate (200 mL). The mixture was vigorously stirred for 1 h and filtered through a plug of Dicalite, washing the solid with DCM. The filtrate layers were separated and the aqueous layer extracted once with DCM. The combined organic layers were dried (MgSO₄), filtered and evaporated to obtain (Z)-3-(3-chlorophenyl)prop-2-en-1-ol (1.20 g, 105% mass recovery), as a light yellow oil. The material was judged pure by LCMS and NMR and used in the next step without further purification. ¹H NMR (400 MHz, CDCl₃, ppm) δ 7.31-7.18 (m, 3H, overlap with CDCl₃), 7.11-7.07 (m, 1H), 6.51 (d, J 12 Hz, 1H), 5.93 (dt, J 12, 6 Hz, 1H), 4.42 (dd, J 6, 2 Hz, 2H).

### Synthesis of (Z)-tert-butyl((3-(3-chlorophenyl)allyl)oxy)dimethylsilane

A solution of tert-butyldimethylsilyl chloride (1.29 g, 8.54 mmol) in DCM (4 mL) was added dropwise to a stirred solution of (Z)-3-(3-chlorophenyl)prop-2-en-1-ol (1.20 g, 6.80 mmol) and imidazole (581 mg, 8.54 mmol) in DCM (35 mL) at 0° C. After addition was complete, the cooling bath was removed, and the suspension was stirred for another 30 min. The mixture was transferred to a separating funnel and washed with water (100 mL). The aqueous layer was extracted once with DCM (10 mL) and the combined organic solutions were dried overnight (MgSO₄), filtered and concentrated in vacuo. The residue was purified on silica gel (40 g) eluting with a gradient of heptane:ethyl acetate=100:0 to 95:5 to afford (Z)-tert-butyl((3-(3-chlorophenyl)allyl)oxy)dimethylsilane (1.93 g, 96%), as a colourless oil. ESI-MS [M−H]⁻ 281. ¹H NMR (400 MHz, CDCl₃, ppm) δ 7.32-7.18 (m, 4H, overlap with CDCl₃), 7.09-7.05 (m, 1H), 6.46-6.40 (m, 1H), 5.88 (dt, J 12, 6 Hz, 1H), 4.39 (dd, J 6, 2 Hz, 2H), 0.90 (s, 9H), 0.06 (s, 6H).

### Synthesis of ethyl (1R*,2R*,3S*) and (1R*,2S*,3R*)-2-(((tert-butyldimethylsilyl)oxy)methyl)-3-(3-chlorophenyl) cyclopropane-1-carboxylate

A solution of (Z)-tert-butyl((3-(3-chlorophenyl)allyl)oxy)dimethylsilane (1.21 g, 4.27 mmol) and Rh₂(OAc)₄ (37 mg, 0.085 mmol) in anhydrous DCM (12 mL) was heated to reflux. Once a steady reflux was established, a solution of ethyl diazoacetate (85% in DCM, 2.11 mL, 17.1 mmol) in anhydrous DCM (6.8 mL) was added via syringe pump over 5 h. At the end of the addition the reaction was judged complete by LCMS, and the solvent was evaporated to obtain an orange oil, which was purified by flash chromatography on silica gel (50 g), eluting with a gradient of heptane:ethyl acetate=100:0 to 90:10 to obtain ethyl (1R*,2R*,3S*) and (1R*,2S*,3R*)-2-(((tert-butyldimethylsilyl)oxy)methyl)-3-(3-chlorophenyl)cyclopropane-1-carboxylate (1:1 mixture of isomers, 1.28 g, containing ca. 6% w/w diethyl fumarate, 77%), as a colourless oil. ESI-MS [M+H]⁺ 369/371; ¹H NMR (400 MHz, CDCl₃, ppm) δ 7.34 (s, 1H), 7.28 (s, 1H), 7.21-7.14 (m, 6H), 4.21-4.15 (m, 2H), 4.09-3.99 (m, 2H), 3.97 (dd, J 11, 8.5 Hz, 1H), 3.90 (dd, J 11, 5 Hz, 1H), 3.59 (dd, J 11, 6 Hz, 1H), 3.26 (dd, J 11, 8 Hz, 1H), 2.83 (dd, J 10, 5 Hz, 1H), 2.72 (t, J 9 Hz, 1H), 2.15 (t, J 9 Hz, 1H), 2.07-2.00 (m, 1H), 1.98 (t, J 5 Hz, 1H), 1.85 (ddd, J 14, 9, 5.4 Hz, 1H), 1.32 (s, J 7 Hz, 3H), 1.18 (t, J 7 Hz, 3H), 0.93 (s, 9H), 0.83 (s, 9H), 0.07 (s, 6H), 0.05 (s, 6H), −0.08 (s, 6H), −0.11 (s, 6H).

### Synthesis of rac-(1R*,2S*,3R*)-2-(3-chlorophenyl)-3-(hydroxymethyl)cyclopropane-1-carboxylic acid and rac-(1R*,2R*,3S*)-2-(3-chlorophenyl)-3-(hydroxymethyl)cyclopropane-1-carboxylic acid

A freshly prepared solution of tetrabutylammonium fluoride trihydrate (2.07 g, 6.56 mmol) in THF (8 mL) was added to a solution of ethyl (1R*,2R*,3S*) and (1R*,2S*,3R*)-2-(((tert-butyldimethylsilyl)oxy)methyl)-3-(3-chlorophenyl)cyclopropane-1-carboxylate (1:1 mixture of isomers, 1.21 g, 3.28 mmol) in THF (8 mL). After 1 h the mixture was diluted with ethyl acetate (100 mL), washed with saturated aqueous sodium bicarbonate (2×50 mL) and brine, dried (MgSO₄), and concentrated under reduced pressure to obtain a dark brown oil that was used in the next step without further treatment.

The mixture of hydroxyesters was dissolved in a mixture of THF (6.4 mL) and methanol (3.2 mL) and aqueous lithium hydroxide (2 M, 6.4 mL, 12.8 mmol) was added at room temperature. After 2 h the reaction was quenched with hydrochloric acid (2 M, 10 mL). Ethyl acetate (100 mL) was added, the layers were separated, and the aqueous layer extracted once with ethyl acetate. The combined organic solutions were washed with brine, dried (Na₂SO₄), filtered and concentrated under reduced pressure to obtain a brown oil (ca. 950 mg). Purification by reversed-phase column chromatography, eluting with a gradient of MeCN:H₂O (50 mL/min, each containing 0.1% formic acid v/v; 10:90 to 35:65 over 12 column volumes). Mixed fractions containing the desired product isomers were pooled and purified again by reversed-phase chromatography (Biotage Cis SNAP Ultra cartridge, 60 g; 50 mL/min, MeCN:H₂O each containing 0.1% formic acid v/v, 10:90 to 55:45 over 12 CV). Fractions containing the separated isomers were pooled to obtain (1R*,2S*,3R*)-2-(3-chlorophenyl)-3-(hydroxymethyl)cyclopropane-1-carboxylic acid as a colourless solid (268 mg, 36%) and (1R*,2R*,3S*)-2-(3-chlorophenyl)-3-(hydroxymethyl)cyclopropane-1-carboxylic acid as an off-white solid (213 mg, 29%). Assignment of the relative stereochemistry was accomplished by 2D NOESY NMR.

(1R*,2S*,3R*)-Isomer

ESI-MS [M−H]⁻ 225/227, ¹H NMR (400 MHz, CD₃OD, ppm) δ 7.33-7.20 (m, 4H), 3.40 (dd, J 12, 7 Hz, 1H), 3.23 (dd, J 12, 7 Hz, 1H), 2.80 (dd, J 10, 5 Hz, 1H), 2.09 (t, J 5 Hz, 1H), 2.03-1.95 (m, 1H).

(1R*,2R*,3S*)-Isomer

ESI-MS [M−H]⁻ 225/227 ¹H NMR (400 MHz, CD₃OD, ppm) δ 7.31-7.17 (m, 4H), 3.96 (dd, J 11, 8 Hz, 1H), 3.85 (dd, J 11, 8 Hz, 1H), 2.78 (t, J 9 Hz, 1H), 2.19 (t, J 9 Hz, 1H), 1.88 (pentet, J 8 Hz, 1H).

### Synthesis of rac-(1R*,2S*,3R*)-2-(3-chlorophenyl)-3-(hydroxymethyl)cyclopropane-1-carboxamide

A mixture of rac-(1R*,2S*,3R*)-2-(3-chlorophenyl)-3-(hydroxymethyl)cyclopropane-1-carboxylic acid (100 mg, 0.44 mmol), (NH₄)₂CO₃ (84.5 mg, 0.88 mmol), HOBt (90.4 mg, 0.66 mmol), EDCI (126.5 mg, 0.66 mmol) and DIPEA (170.3 mg, 1.32 mmol) in DMF (5 mL) was stirred at room temperature for 18 h. Water (20 mL) was added then the reaction was extracted with EtOAc (3×30 mL). The combined organic layers were washed with brine (30 mL), dried over Na₂SO₄, filtered and concentrated in vacuo to give the crude, which was purified by silica gel chromatography (eluant: DCM/MeOH=20/1) to give rac-(1R*,2S*,3R*)-2-(3-chlorophenyl)-3-(hydroxymethyl)cyclopropane-1-carboxamide (38 mg, 38%) as a white solid. ESI-MS [M+H]+: 226.1.

### Synthesis of rac-(1S*,2S*)-2-(5-methoxy-2-oxopyridin-1(2H)-yl)cyclopropane-1-carboxamide

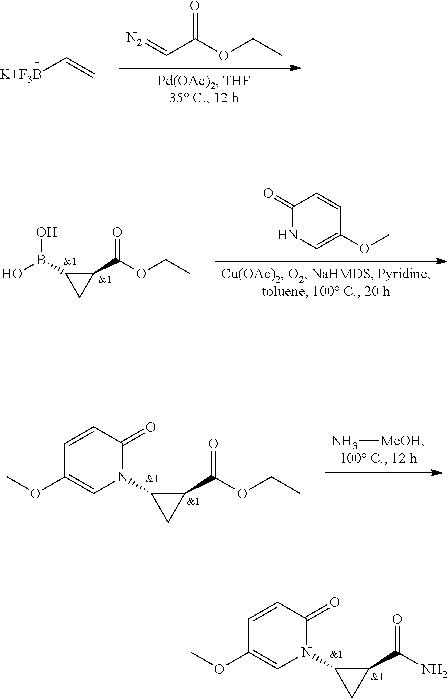

### Synthesis of rac-((1S*,2S*)-2-(ethoxycarbonyl)cyclopropyl)boronic acid

A mixture of trifluoro(vinyl)borate (3.0 g, 22.5 mmol), ethyl 2-diazoacetate (5.1 g, 45 mmol) and Pd(OAc)₂ (505 mg, 2.25 mmol) in THF (30 ml) was stirred at 35° C. for 12 h. The reaction mixture was filtered and the filter cake was washed by DCM (400 ml). The filtrate was concentrated in vacuo to give the crude rac-((1S*,2S*)-2-(ethoxycarbonyl)cyclopropyl)boronic acid (9.0 g crude), which was used into next step without further purification. ESI-MS: [M+H]⁺, 159.2

### Synthesis of rac-ethyl (1S*,2S*)-2-(5-methoxy-2-oxopyridin-1(2H)-yl)cyclopropane-1-carboxylate

A mixture of rac-((1S*,2S*)-2-(ethoxycarbonyl)cyclopropyl)boronic acid (312 mg, 2.5 mmol), 5-methoxypyridin-2(1H)-one (1.18 g, 7.5 mmol), pyridine (987 mg, 12.5 mmol), NaHMDS (1.25 ml, 2.5 mmol) and Cu(OAc)₂ (452 mg, 2.5 mmol) in toluene (10 ml) was stirred at 100° C. for 20 h. The mixture was cooled to room temperature, filtered and the filter cake was washed by EtOAc (100 mL). The filtrate was concentrated in vacuo to give the crude, which was purified by silica gel chromatography (eluant: EtOAc/PE=4/1) to afford rac-ethyl (1S*,2S*)-2-(5-methoxy-2-oxopyridin-1(2H)-yl)cyclopropane-1-carboxylate (125 mg, 20%) as a yellow oil. ESI-MS: [M+H]⁺, 238.2

### Synthesis of rac-(1S*,2S*)-2-(5-methoxy-2-oxopyridin-1(2H)-yl)cyclopropane-1-carboxamide

A mixture of rac-ethyl (1S*,2S*)-2-(5-methoxy-2-oxopyridin-1(2H)-yl)cyclopropane-1-carboxylate (100 mg, 0.1 mmol) and NH₃ in MeOH (7M, 10 ml, 70 mmol) were stirred at 100° C. for 12 h in a sealed tube. The reaction was cooled to room temperature and concentrated in vacuo to give the crude which was purified by preparative TLC (eluant: DCM:MeOH=10:1) to afford rac-(1S*,2S*)-2-(5-methoxy-2-oxopyridin-1(2H)-yl)cyclopropane-1-carboxamide (65 mg, 75%) as a white solid. ESI-MS: [M+H]⁺, 209.2

### Synthesis of rac-(1S*,2S*)-2-(4-methoxypyridin-2-yl)cyclopropane-1-carboxamide

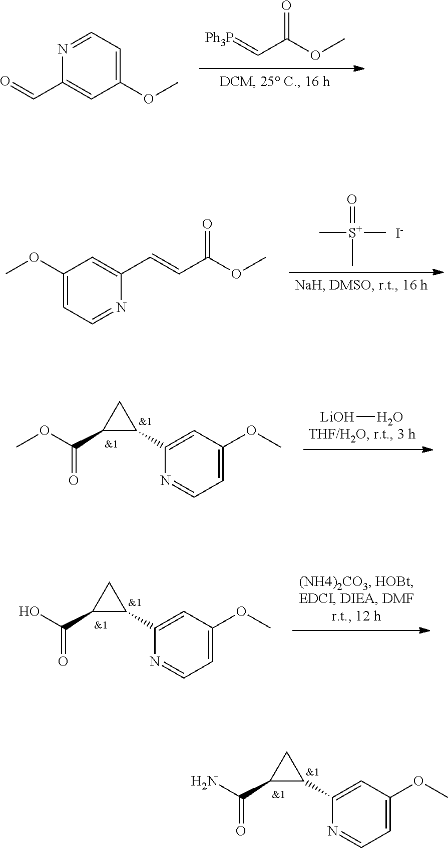

### Synthesis of methyl (E)-3-(4-methoxypyridin-2-yl)acrylate

A mixture of 4-methoxypicolinaldehyde (3 g, 21.9 mmol) and methyl 2-(triphenyl-15-phosphaneylidene)acetate (7.3 g, 21.9 mmol) in DCM (30 mL) was stirred at room temperature for 16 h. The reaction was quenched with water (100 mL) then extracted with DCM (3×50 mL). The combined organic layers were washed with brine (60 mL), dried over Na₂SO₄ and concentrated in vacuo. The residue was purified by silica gel column chromatography (eluent: EtOAc/PE from 0 to 20%) to afford methyl (E)-3-(4-methoxypyridin-2-yl)acrylate (3.3 g, 79% yield) as a white solid. ESI-MS: [M+H]⁺, 194.2

### Synthesis of rac-methyl (1S*,2S*)-2-(4-methoxypyridin-2-yl)cyclopropane-1-carboxylate

To a mixture of trimethylsulfoxonium iodide (6.9 g, 31.2 mmol) in DMSO (50 mL) was added NaH (1.2 g, 60% suspension in paraffin oil, 31.2 mmol). The resulting mixture was stirred at room temperature for 2 h. A solution of methyl (E)-3-(4-methoxypyridin-2-yl)acrylate (2.0 g, 10.4 mmol) in DMSO (10 mL) was added and reaction mixture was stirred at room temperature for 16 h. The reaction was quenched with saturated NH₄Cl (60 mL) then extracted with EtOAc (3×100 mL). The combined organic layers were washed with brine (100 mL), dried over Na₂SO₄ and concentrated. The residue was purified by silica gel column chromatography (eluent: EtOAc/PE from 0 to 20%) to afford rac-methyl (1S*,2S*)-2-(4-methoxypyridin-2-yl)cyclopropane-1-carboxylate (1.3 g, 60% yield) as a yellow oil. ESI-MS: [M+H]⁺, 208.2

### Synthesis of rac-(1S*,2S*)-2-(4-methoxypyridin-2-yl)cyclopropane-1-carboxamide

Using a similar procedure to that for 2-(5-chloro-2-nitrophenyl)cyclopropane-1-carboxamide, rac-(1S*,2S*)-2-(4-methoxypyridin-2-yl)cyclopropane-1-carboxamide (0.65 g, 50%) was synthesised from rac-methyl (1S*,2S*)-2-(4-methoxypyridin-2-yl)cyclopropane-1-carboxylate (1.3 g, 6.3 mmol). ESI-MS: [M+H]⁺, 193.2.

### Synthesis of rac-tert-butyl (4-((1S*,2S*)-2-carbamoylcyclopropyl)-6-methoxypyridin-2-yl)carbamate

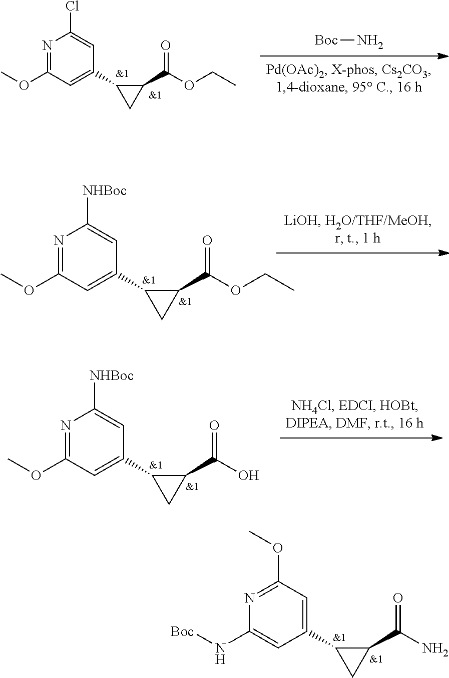

### Synthesis of rac-ethyl (1S*,2S*)-2-(2-((tert-butoxycarbonyl)amino)-6-methoxypyridin-4-yl)cyclopropane-1-carboxylate

A mixture of rac-ethyl (1S*,2S*)-2-(2-chloro-6-methoxypyridin-4-yl)cyclopropane-1-carboxylate (300 mg, 1.17 mmol), tert-butyl carbamate (1.1 g, 9.36 mmol), Pd(OAc)₂ (26 mg, 0.117 mmol), X-phos (112 mg, 0.234 mmol) and Cs₂CO₃ (1.14 g, 3.51 mmol) in 1,4-dioxane (10 mL) was stirred at 90° C. for 16 h under N₂. The reaction mixture was cooled to room temperature, filtered through Celite®, and then the filter cake was washed with DCM/MeOH (10/1, 50 mL). The filtrate was concentrated in vacuo to give the crude, which was purified by silica gel chromatography (eluent: EtOAc/PE=1/30) to give rac-ethyl (1S*,2S*)-2-(2-((tert-butoxycarbonyl)amino)-6-methoxypyridin-4-yl)cyclopropane-1-carboxylate (320 mg, 81%) as a white solid. ESI-MS [M+H]⁺: 337.1.

### Synthesis of rac-(1S*,2S*)-2-(2-((tert-butoxycarbonyl)amino)-6-methoxypyridin-4-yl)cyclopropane-1-carboxylic acid

A mixture of rac-ethyl (1S*,2S*)-2-(2-((tert-butoxycarbonyl)amino)-6-methoxypyridin-4-yl)cyclopropane-1-carboxylate (320 mg, 0.951 mmol) and LiOH—H₂O (160 mg, 3.81 mmol) in THF (5 mL)/MeOH (5 mL)/H₂O (4 mL) was stirred at room temperature for 2 h. The reaction mixture was poured into water (40 mL) then the pH of the was acidified to 4˜5 with HCl (2 N). The reaction mixture was extracted with DCM/MeOH (10/1, 3×40 ml). The combined organics were washed with brine (50 mL), dried over Na₂SO₄ then concentrated in vacuo to give rac-(1S*,2S*)-2-(2-((tert-butoxycarbonyl)amino)-6-methoxypyridin-4-yl)cyclopropane-1-carboxylic acid (293 mg crude) as a white solid. ESI-MS [M+H]+: 309.1.

### Synthesis of rac-tert-butyl (4-((1S*,2S*)-2-carbamoylcyclopropyl)-6-methoxypyridin-2-yl)carbamate

A mixture of rac-(1S*,2S*)-2-(2-((tert-butoxycarbonyl)amino)-6-methoxypyridin-4-yl)cyclopropane-1-carboxylic acid (293 mg, 0.951 mmol), NH₄Cl (509 mg, 9.51 mmol), EDCI (365 mg, 1.90 mmol), HOBt (257 mg, 1.90 mmol) and DIPEA (737 mg, 5.71 mmol) in DMF (10 mL) was stirred at 25° C. for 16 h. The reaction mixture was poured into water (30 mL) then extracted with EtOAc (3×40 ml). The combined organics were washed with brine (40 mL), dried over Na₂SO₄ then concentrated in vacuo to give the crude, which was purified by silica gel chromatography (eluent: EtOAc/PE=1/1) to give rac-tert-butyl (4-((1S*,2S*)-2-carbamoylcyclopropyl)-6-methoxypyridin-2-yl)carbamate (240 mg, 82%) as a white solid. ESI-MS [M+H]⁺: 308.1.

### Synthesis of rac-(1R*,2R*)-6′-chloro-2′,3′-dihydrospiro[cyclopropane-1,1′-indene]-2-carboxamide

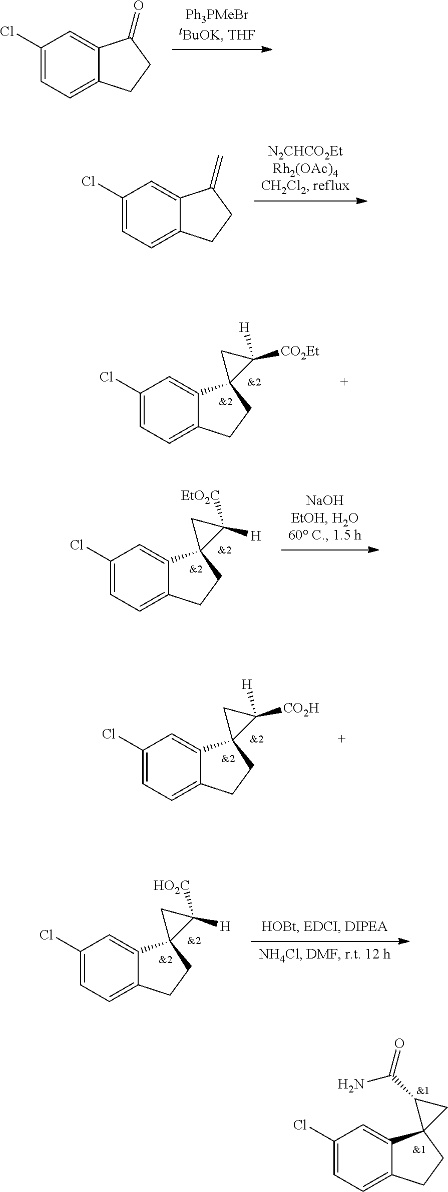

### Synthesis of 6-chloro-1-methylene-2,3-dihydro-1H-indene

A solution of potassium tert-butoxide (97.0 mL, 1.0 M in THF, 97.0 mmol) was added to a suspension of methyltriphenylphosphonium bromide (34.7 g, 97.1 mmol) in anhydrous THF (150 mL) under nitrogen at 0° C. The bright yellow suspension was stirred for 1 h and a solution of 6-chloroindan-1-one (8.09 g, 48.6 mmol) in anhydrous THF (75 mL) was added over 5 min. Once the addition was complete the cooling bath was removed. After 1 h, saturated aqueous ammonium chloride (100 mL) was added slowly. After being stirred for 10 min, the THF was removed in vacuo, and the residue was diluted with ethyl acetate (150 mL) and water (100 mL). The layers were separated, and the organic phase washed with brine, dried (MgSO₄) and concentrated in vacuo onto silica gel (50 g). This material was applied to the top of a chromatography column (200 g silica gel) which was eluted with heptane to afford 6-chloro-1-methylene-2,3-dihydro-1H-indene (7.24 g, 90%) as a light-yellow oil. ¹H NMR (400 MHz, CDCl₃, ppm): δ 7.44 (s, 1H), 7.18-7.16 (m, 2H), 5.44 (t, J 2 Hz, 1H), 5.07 (t, J 2 Hz, 1H), 2.95-2.92 (m, 2H), 2.84-2.80 (m, 2H).

### Synthesis of ethyl (1R*,2R*)-6′-chloro-2′,3′-dihydrospiro[cyclopropane-1,1′-indene]-2-carboxylate and ethyl (1R*,2S*)-6′-chloro-2′,3′-dihydrospiro[cyclopropane-1,1′-indene]-2-carboxylate

A solution of ethyl diazoacetate (85%, 10.0 g, 104 mmol) in anhydrous DCM (20 mL) was added via syringe pump over 5 h to a stirred solution of 6-chloro-1-methylene-2,3-dihydro-1H-indene (5.69 g, 34.6 mmol) and rhodium acetate (153 mg, 0.346 mmol) in anhydrous DCM (80 mL) under nitrogen at reflux. The dark green-blue mixture was concentrated in vacuo, dissolved in toluene and applied to the top of a silica gel column (500 g), which was eluted with 1-4% methyl tert-butyl ether in heptane in 1% increments to give a mixture of ethyl (1R*,2R*)-6′-chloro-2′,3′-dihydrospiro[cyclopropane-1,1′-indene]-2-carboxylate and ethyl (1R*,2S*)-6′-chloro-2′,3′-dihydrospiro[cyclopropane-1,1′-indene]-2-carboxylates (ratio 6:5, 3.932 g, 45%). Purification of several aliquots (10×7 mg) of the isomeric mixture by preparative HPLC was performed:

First eluted was a colourless oil (10 mg, RT=5.6 min, minor isomer). This was assigned as the (1R*,2S*) by n.O.e experiments. ¹H NMR (400 MHz, CDCl₃, ppm): δ 7.18 (s, 1H), 7.10 (m, 2H), 4.06 (dq, J 11, 7 Hz, 1H), 3.99 (dq, J 11, 7 Hz, 1H), 3.05 (ddd, J 8.2, 10, 16 Hz, 1H), 2.86 (ddd, J 1.5, 8.9, 16 Hz, 1H), 2.39 (ddd, J 8.9, 10, 13 Hz, 1H), 2.10 (dd, J 6.4, 8 Hz, 1H), 1.92 (ddd, J 1.5, 8.2, 13 Hz, 1H), 1.84 (dd, J 5.6, 6.4 Hz, 1H), 1.41 (dd, J 5.6, 8 Hz, 1H), 1.16 (t, J 7 Hz, 3H).

Second eluted was a colourless oil (16 mg, RT=6.0 min, major isomer). This was assigned as the (1R*,2R*) by n.O.e experiments. ¹H NMR (400 MHz, CDCl₃, ppm): δ 7.10 (m, 2H), 6.65 (s, 1H), 4.15 (m, 2H), 2.96 (m, 2H), 2.27 (m, 2H), 2.00 (dd, J 6, 8.5 Hz, 1H), 1.65 (dd, J 5, 6 Hz, 1H), 1.39 (dd, J 5, 8.5 Hz, 1H), 1.25 (t, J 7.2 Hz, 3H).

### Synthesis of (1R*,2R*)-6′-chloro-2′,3′-dihydrospiro[cyclopropane-1,1′-indene]-2-carboxylic acid and (1R*,2S*)-6′-chloro-2′,3′-dihydrospiro[cyclopropane-1,1′-indene]-2-carboxylic acid

A solution of sodium hydroxide (1.88 g, 46.9 mmol) in water (23 mL) was treated with a solution of a mixture of ethyl (1R*,2R*)-6′-chloro-2′,3′-dihydrospiro[cyclopropane-1,1′-indene]-2-carboxylate and ethyl (1R*,2S*)-6′-chloro-2′,3′-dihydrospiro[cyclopropane-1,1′-indene]-2-carboxylates (ratio 6:5, 3.92 g, 15.6 mmol) in ethanol (75 mL) under nitrogen. The mixture was heated at 60° C. for 90 min then was cooled to room temperature, concentrated in vacuo, and the residue was diluted with water (70 mL). Hydrochloric acid (2M) was added to give pH 1 which precipitated a gummy solid. The gum was extracted into ethyl acetate (3×70 mL), which was dried (Na₂SO₄) and concentrated in vacuo to give a pale brown solid. Recrystallisation from a mixture of boiling heptane (70 mL) and ethyl acetate (20 mL) gave (1S*,2S*)-6′-chloro-2′,3′-dihydrospiro[cyclopropane-1,1′-indene]-2-carboxylic acid (1169 mg, 30%), as colourless needles. ESI-MS [M−H]⁻ 221/223, ¹H NMR (400 MHz, DMSO, ppm): δ 12.28 (br s, 1H), 7.22 (d, J 8 Hz, 1H), 7.17 (dd, J 8, 1.7 Hz, 1H), 6.98 (d, J 1.7 Hz, 1H), 2.99-2.86 (m, 2H), 2.24-2.11 (m, 2H), 2.00 (dd, J 6, 8.5 Hz, 1H), 1.48 (dd, J 5, 8.5 Hz, 1H), 1.44 (dd, J 5, 6 Hz, 1H).

### Synthesis of rac-(1S*,2S*)-6′-chloro-2′,3′-dihydrospiro[cyclopropane-1,1′-indene]-2-carboxamide

A mixture of rac-(1S*,2S*)-6′-chloro-2′,3′-dihydrospiro[cyclopropane-1,1′-indene]-2-carboxylic acid (230 mg, 1.04 mmol), NH₄Cl (220 mg, 4.14 mmol), HOBt (351 mg, 2.6 mmol), EDCI (500 mg, 2.6 mmol) and DIPEA (671 mg, 5.2 mmol) in DMF (15 mL) was stirred at room temperature for 12 h under N₂. The reaction was quenched with water (30 mL) then extracted with EtOAc (3×30 mL). The combined organic layers were washed with brine (50 mL), dried over Na₂SO₄, then concentrated in vacuo to give the crude, which was triturated with DCM (10 mL) to give rac-(1S*,2S*)-6′-chloro-2′,3′-dihydrospiro[cyclopropane-1,1′-indene]-2-carboxamide (180 mg, 78%) as a white solid. ESI-MS [M+H]+: 222.1

### Synthesis of rac-(1S*,2S*)-2-(3-chlorophenyl)-1-fluorocyclopropane-1-carboxamide

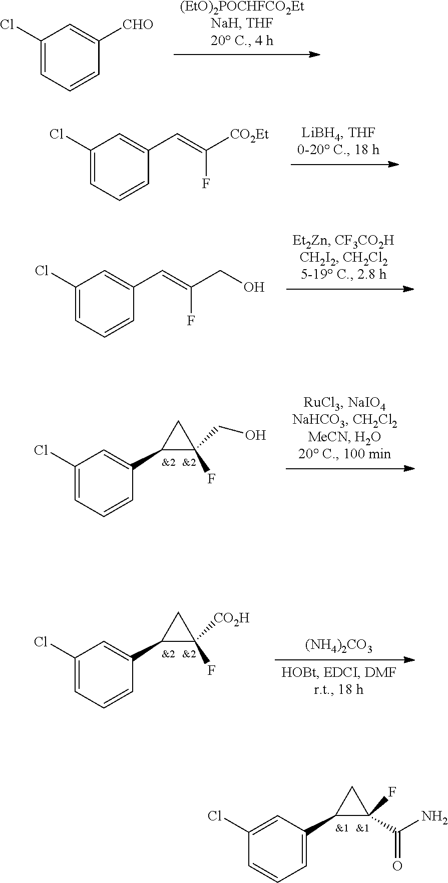

### Synthesis of ethyl Z-(3-chlorophenyl)-2-fluoropropanoate

A solution of triethyl 2-fluorophosphonoacetate (12.1 g, 50 mmol) in anhydrous THF (25 mL) was added dropwise via syringe over 40 min to a stirred suspension of sodium hydride (2.00 g, 60% oil dispersion, 50 mmol) in anhydrous THF (50 mL) under nitrogen at room temperature. The resulting orange mixture was stirred for 55 min, and a solution of 3-chlorobenzaldehyde (2.81 g, 20.0 mmol) in anhydrous THF (15 mL) was added dropwise over 30 min. The resulting mixture was stirred for 4 h, and then treated with aqueous ammonium chloride (10% w/v, 75 mL). The product was extracted into methyl tert-butyl ether (100 mL and 50 mL) and the organic solutions were washed with water (2×50 mL), dried (Na₂SO₄) and concentrated in vacuo. The residue was dissolved in a little heptane (with warming) and purified by flash chromatography on silica gel (150 g), eluting with heptane followed by heptane:ethyl acetate=50:1. Fractions containing product were combined and concentrated in vacuo to give a colourless solid (3.371 g, 74%), which consisted of a 5:1 mixture of E and Z isomers.

Following the discovery that the desired Z-isomer is a solid and the undesired E-isomer is a liquid, the 5:1 mixture of isomers (2.79 g) was suspended in 7 mL heptane and stored for 24 h at −20° C. The crystals were collected by filtration and washed with cold heptane to give ethyl Z-(3-chlorophenyl)-2-fluoropropanoate (2.017 g), as a colourless solid.

¹H NMR (400 MHz, CDCl₃, ppm) Z-isomer: δ 7.64 (s, 1H), 7.52-7.50 (m, 1H), 7.36-7.31 (m, 2H), 6.85 (d, J 34 Hz, 1H), 4.36 (q, J 7.2 Hz, 2H), 1.39 (t, J 7.2 Hz, 3H).

¹H NMR (400 MHz, CDCl₃, ppm) E-isomer: δ 7.47 (s, 1H), 7.32-7.28 (m, 3H), 6.84 (d, J 22 Hz, 1H), 4.25 (q, J 7.2 Hz, 2H), 1.25 (t, J 7.2 Hz, 3H).

¹⁹F NMR (373 MHz, CDCl₃, ppm, no reference) δ −115.3 (d, J 21 Hz, minor isomer), −122.9 (d, J 34 Hz, major isomer).

### Synthesis of (Z)-3-(3-chlorophenyl)-2-fluoroprop-2-en-1-ol

Lithium borohydride (789 mg, 37.6 mmol) was added in portions to a stirred solution of Z-(3-chlorophenyl)-2-fluoropropanoate (2.147 g, 9.40 mmol) in anhydrous THF (50 mL) under nitrogen with cooling in an ice-water bath. After 10 min, the bath was removed, and the mixture was stirred at room temperature 17.5 h. The mixture was cooled in an ice-water bath and treated with saturated aqueous ammonium chloride (25 mL). Ethyl acetate (30 mL) and water (30 mL) were added and the layers were separated. The aqueous layer was extracted with ethyl acetate (3×50 mL) and the combined extracts were washed with brine (30 mL), dried (Na₂SO₄) and concentrated in vacuo. The residue was dissolved in ethyl acetate and adsorbed onto Dicalite. Purification by flash chromatography (100 g silica gel) eluting with heptane:ethyl acetate=3:1 gave (Z)-3-(3-chlorophenyl)-2-fluoroprop-2-en-1-ol, as a colourless oil (1.840 g, containing 10% w/w ethyl acetate, 95%), which crystallised on standing. ¹H NMR (400 MHz, CDCl₃, ppm) δ 7.52 (s, 1H), 7.36 (d, J 7.6 Hz, 1H), 7.25 (t, J 8 Hz, 1H), 7.21 (m, 1H), 5.75 (d, J 38 Hz, 1H), 4.30 (dd, J 7, 13.5 Hz, 2H), 1.85 (t, J 7 Hz, 1H). ¹⁹F NMR (373 MHz, CDCl₃, ppm, no reference) δ −111.1 (dt, J 38, 13.5 Hz).

### Synthesis of ((1S*,2S*)-2-(3-chlorophenyl)-1-fluorocyclopropyl)methanol

A solution of trifluoroacetic acid (2.58 mL, 33.5 mmol) in anhydrous DCM (5 mL) was added dropwise over 17 min to a solution of diethyl zinc (33.5 mL, 1.0 M in hexanes, 33.5 mmol) in anhydrous DCM (66 mL) under nitrogen, keeping the internal temperature between 5° C. and 7° C. by means of an ice-water bath. After 20 min, a solution of diiodomethane (2.70 mL, 33.5 mmol) in anhydrous DCM (5 mL) was added dropwise over 12 min (internal temperature 5° C.). The mixture was stirred for 26 min and then a solution of (Z)-3-(3-chlorophenyl)-2-fluoroprop-2-en-1-ol (1.561 g, 8.37 mmol) in anhydrous DCM (10 mL) was added dropwise over 12 min (internal temperature 3-5° C.). After another 12 min the cooling bath was taken away, and the mixture was allowed to warm to room temperature. The mixture was stirred for 2.8 h, treated with treated with saturated aqueous ammonium chloride (25 mL), initially dropwise. Water (50 mL) was added and the layers were separated. The aqueous layer was extracted with DCM (3×40 mL) and the combined extracts were dried (Na₂SO₄) and concentrated in vacuo. Purification by flash chromatography (100 g silica gel) eluting with heptane:ethyl acetate=4:1 gave ((1S*,2S*)-2-(3-chlorophenyl)-1-fluorocyclopropyl)methanol, as an almost colourless oil (1.169 g, 70%). ¹H NMR (400 MHz, CDCl₃, ppm) δ 7.26-7.20 (m, 3H), 7.15-7.13 (m, 1H), 4.01 (ddd, J 5.6, 13, 20 Hz, 1H), 3.86 (ddd, J 6.4, 13, 20 Hz, 1H), 2.21 (ddd, J 4.4, 8, 10.4 Hz, 1H), 1.94-1.90 (m, 1H), 1.49 (dt, J 20, 8 Hz, 1H), 1.32 (dt, J 20, 8 Hz, 1H). ¹⁹F NMR (376 MHz, CDCl₃, ppm, no reference) δ −200.2 (dddt, J 4, 11, 13, 20 Hz).

### Synthesis of (1S*,2S*)-2-(3-chlorophenyl)-1-fluorocyclopropane-1-carboxylic acid

((1S*,2S*)-2-(3-Chlorophenyl)-1-fluorocyclopropyl)methanol (1.160 g, 5.79 mmol) was dissolved in a mixture of DCM (40 mL) and acetonitrile (40 mL) under nitrogen at room temperature. Water (60 mL) was added followed by sodium bicarbonate (3.205 g, 38.2 mmol) and sodium meta-periodate (6.946 g, 32.5 mmol). Finally, ruthenium (III) chloride (240 mg, 1.16 mmol) was added and the mixture was stirred vigorously for 100 min. The mixture was diluted with DCM (30 mL) and water (30 mL) and acidified with 2M hydrochloric acid to pH1. The mixture was filtered through a pad of Dicalite, and the filter cake was washed with DCM (100 mL). The filtrate layers were separated, the aqueous layer was extracted with DCM (2×50 mL) and the combined extracts were dried (Na₂SO₄) and concentrated in vacuo. The resulting black oily residue was adsorbed onto silica gel (10 g) with DCM and purified by flash chromatography, eluting with heptane:ethyl acetate:acetic acid=50:50:1 to give (1R*,2R*)-2-(3-chlorophenyl)-1-fluorocyclopropane-1-carboxylic acid, as a pale grey oil (912 mg, 73%). This material was combined with another batch of similar quality (96 mg) and re-purified by flash chromatography on silica gel (100 g), eluting with heptane:ethyl acetate:acetic acid=50:50:1 to give (1S*,2S*)-2-(3-chlorophenyl)-1-fluorocyclopropane-1-carboxylic acid, as pale grey oil (1043 mg, 68%, containing the following residual solvents: acetic acid (6.1% w/w), DCM (2.7% w/w) and ethyl acetate (1.3% w/w)). ESI-MS [M−H]⁻ 213/215, ¹H NMR (400 MHz, CDCl₃, ppm) δ 7.28-7.26 (m, 3H), 7.16-7.14 (m, 1H), 2.91 (ddd, J 2.4, 9.2, 11.2 Hz, 1H), 2.00 (ddd, J 2.4, 7.2, 10 Hz, 1H), 1.87 (ddd, J 7.2, 9.2, 16 Hz, 1H). ¹⁹F NMR (373 MHz, CDCl₃, ppm, no reference) δ −207.0 (apparent dd, J 10, 17 Hz).

### Synthesis of rac-(1S*,2S*)-2-(3-chlorophenyl)-1-fluorocyclopropane-1-carboxamide

A mixture of rac-(1S*,2S*)-2-(3-chlorophenyl)-1-fluorocyclopropane-1-carboxylic acid (370 mg, 1.72 mmol), (NH₄)₂CO₃ (331 mg, 3.45 mmol), HOBt (348.3 mg, 2.58 mmol), EDCI (497.9 mg, 2.58 mmol) and DIPEA (665.6 mg, 5.16 mmol) in DMF (5 mL) was stirred at room temperature for 18 h. Water (20 mL) was added then the mixture was extracted with EtOAc (3×30 mL). The combined organic layers were washed with brine (20 mL), dried over Na₂SO₄, filtered and concentrated in vacuo to give the crude, which was purified by silica gel chromatography (eluant: PE/EtOAc=2/1) to give rac-(1S*,2S*)-2-(3-chlorophenyl)-1-fluorocyclopropane-1-carboxamide (180 mg, 49.2%) as a white solid. ESI-MS [M+H]+: 214.1.

### Synthesis of rac-(1S*,2S*)-2-(5-chlorothiophen-2-yl)cyclopropane-1-carboxamide

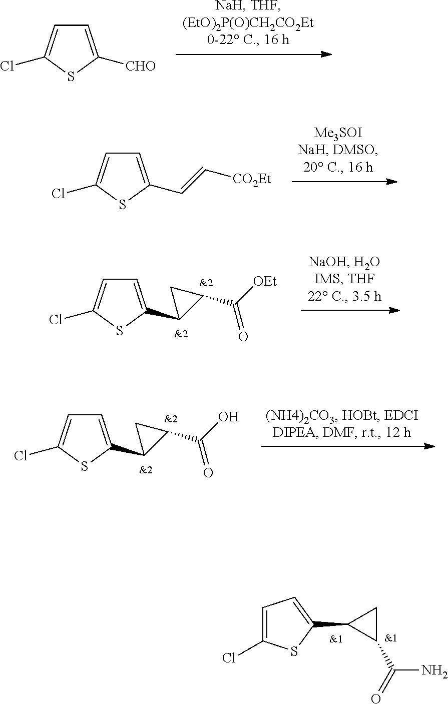

### Synthesis of ethyl (E)-3-(5-chlorothiophen-2-yl)acrylate

Sodium hydride (60% dispersion in oil, 1.074 g, 26.9 mmol) was suspended in anhydrous THF (150 mL) at 0° C. under nitrogen. Triethyl phosphonoacetate (4.80 mL, 24.2 mmol) was added dropwise over 5 min. The mixture was stirred for 45 min. 5-Chloro-2-thiophenecarboxaldehyde (2.20 mL, 20.7 mmol) was added dropwise over 5 min. The mixture was allowed to warm to room temperature and stirred for a further 16 h. The mixture was poured into saturated aqueous ammonium chloride (300 mL) and extracted with methyl tert-butyl ether (3×100 mL). The combined extracts were washed with brine (150 mL), dried (MgSO₄), filtered and concentrated in vacuo to give a light brown oil (5.055 g). Purification by flash column chromatography (SiliCyCle SiliaSep cartridge, 120 g) eluting with 5% ethyl acetate in heptane for 1 column volume followed by 5-25% ethyl acetate in heptane over 10 column volumes gave ethyl (E)-3-(5-chlorothiophen-2-yl)acrylate (3.585 g, 80%) as a pale yellow oil. ¹H NMR (400 MHz, CDCl₃, ppm) δ 7.63 (d, J 15.7 Hz, 1H), 7.02 (d, J 4.2 Hz, 1H), 6.87 (d, J 4.2 Hz, 1H), 6.10 (d, J 15.7 Hz, 1H), 4.24 (q, J 7.0 Hz, 2H), 1.32 (t, J 7.0 Hz, 3H).

### Synthesis of ethyl (1R*,2R*)-2-(5-chlorothiophen-2-yl)cyclopropane-1-carboxylate

Sodium hydride (60% dispersion in oil, 0.381 g, 9.53 mmol) was washed with heptane (2×10 mL) under nitrogen prior to use and then anhydrous DMSO (12 mL) was added. To this was added a solution of trimethylsulfoxonium iodide (2.058 g, 9.35 mmol) in anhydrous DMSO (14 mL) dropwise over 15 min to give a cloudy suspension. The mixture was stirred at room temperature for 1 h, during which time the mixture became a pale-yellow solution. To this was added a solution of (E)-3-(5-chlorothiophen-2-yl)acrylate (1.350 g, 6.23 mmol) in anhydrous DMSO (4 mL) dropwise over 5 min. The mixture was stirred for 16 h and then poured into water (50 mL) and extracted with ethyl acetate (4×30 mL). The combined extracts were washed with water (2×50 mL) and brine (50 mL). The extracts were dried (Na₂SO₄), filtered and concentrated in vacuo to give an orange oil. Purification by flash column chromatography (SiliCyCle SiliaSep cartridge, 40 g) eluting with 5% ethyl acetate in heptane for 1 column volume followed by 5-30% ethyl acetate in heptane over 15 column volumes gave ethyl (1R*,2R*)-2-(5-chlorothiophen-2-yl)cyclopropane-1-carboxylate (0.496 g, 35%) as a pale yellow oil. ¹H NMR (400 MHz, CDCl₃, ppm) δ 6.69 (d, J 4.2 Hz, 1H), 6.58 (d, J 4.4 Hz, 1H), 4.17 (q, J 7.1 Hz, 2H), 2.58 (ddd, J 9, 5, 4 Hz, 1H), 1.88 (ddd, J 8, 5, 4 Hz, 1H), 1.58 (ddd, J 9, 6, 5 Hz, 1H), 1.28 (t, J 7.1 Hz, 3H), 1.25 (ddd, J 8, 6, 4 Hz, 1H).

### Synthesis of (1R*,2R*)-2-(5-chlorothiophen-2-yl)cyclopropane-1-carboxylic acid

Ethyl (1R*,2R*)-2-(5-chlorothiophen-2-yl)cyclopropane-1-carboxylate (0.643 g, 2.79 mmol) was dissolved in a mixture of aqueous sodium hydroxide (2 M, 3.5 mL, 7.00 mmol), THF (3 mL) and industrial methylated spirit (3 mL). The mixture was stirred at room temperature for 2 h 15 min and then acidified to pH 5 using aqueous hydrochloric acid (2 M). The mixture was diluted with water (10 mL) and extracted with ethyl acetate (3×20 mL). The combined extracts were washed with water (20 mL) and brine (20 mL), dried (MgSO₄), filtered and concentrated in vacuo to give a pale-yellow oil. The aqueous phase was found to contain product and so the pH was reduced to pH 3 with hydrochloric acid. The aqueous phase was extracted further with ethyl acetate (3×20 mL), and the extracts were dried (MgSO₄), filtered and concentrated in vacuo to give a pale-yellow oil. The two oils (ca. 0.7 g) were combined and purified by column chromatography on silica gel (21 g) eluting with 10% ethyl acetate in heptane+1% acetic acid to give (1R*,2R*)-2-(5-chlorothiophen-2-yl)cyclopropane-1-carboxylic acid (0.549 g, 84%) as a pale-yellow oil that solidified on standing. ESI-MS [M−H]⁻ 201, H NMR (400 MHz, CDCl₃, ppm) δ 6.71 (d, J 3.6 Hz, 1H), 6.60 (d, J 3.0 Hz, 1H), 2.67 (ddd, J 10, 7, 4 Hz, 1H), 1.90 (ddd, J 9, 5, 4 Hz, 1H), 1.65 (ddd, J 10, 5, 5 Hz, 1H), 1.36 (ddd, J 9, 7, 5 Hz, 1H).

### Synthesis of rac-(1S*,2S*)-2-(5-chlorothiophen-2-yl)cyclopropane-1-carboxamide

A solution of rac-(1S*,2S*)-2-(5-chlorothiophen-2-yl)cyclopropane-1-carboxylic acid (260 mg, 1.28 mmol), (NH₄)₂CO₃ (247 mg, 2.56 mmol), HOBt (260 mg, 1.92 mmol), EDCI (372 mg, 1.92 mmol), and DIPEA (498 mg, 3.84 mmol)) in DMF (4 mL) was stirred at room temperature for 12 h. Water (20 mL) was added to the reaction which was then extracted with EtOAc (5×50 mL). The combined organic layers were washed with brine (30 mL), dried over anhydrous Na₂SO₄, and concentrated in vacuo to give the crude, which was purified by silica gel chromatography (eluent: DCM/MeOH=15/1) to give rac-(1S*,2S*)-2-(5-chlorothiophen-2-yl)cyclopropane-1-carboxamide (180 mg, 69%) as a white solid. ESI-MS [M+H]+: 202.1

### Synthesis of rac-(1S*,2S*)-2-(4-chlorothiophen-2-yl)cyclopropane-1-carboxamide

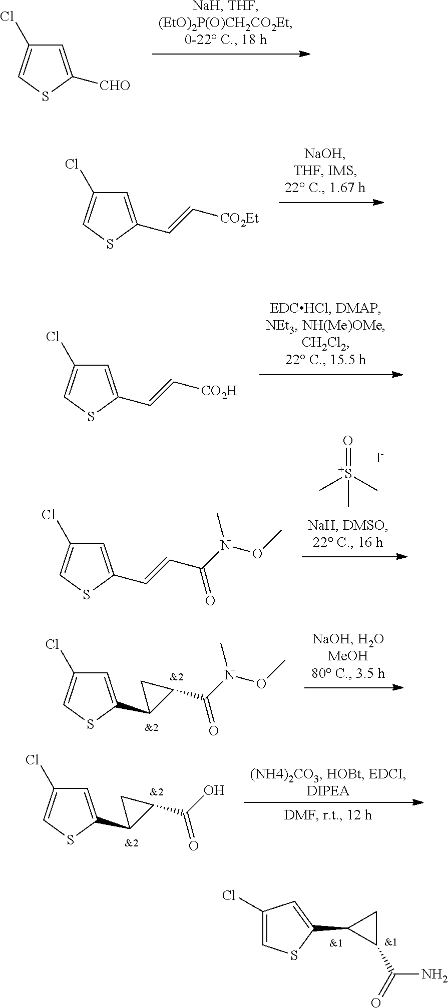

### Synthesis of ethyl (E)-3-(4-chlorothiophen-2-yl)acrylate

Sodium hydride (60% dispersion in oil, 1.074 g, 26.9 mmol) was suspended in anhydrous THF (150 mL) under nitrogen at 0° C. Triethyl phosphonoacetate (4.80 mL, 24.2 mmol) was added dropwise over 5 min and the mixture was stirred for 1 h. 4-Chloro-2-thiophenecarboxaldehyde (2.20 mL, 20.7 mmol) was added dropwise over 5 min. The mixture was warmed to room temperature and stirred for a further 18 h. The reaction mixture was poured into saturated aqueous ammonium chloride (300 mL) and extracted with methyl tert-butyl ether (3×100 mL). The combined organic extracts were washed with brine (150 mL), dried (MgSO₄), filtered and concentrated in vacuo to give a dark red solid (5.156 g). The solid was purified by silica gel column chromatography (100 g), eluting with 10% ethyl acetate in heptane to give ethyl (E)-3-(4-chlorothiophen-2-yl)acrylate (3.355 g, 75%), as an orange solid. ESI-MS [M−OEt]⁺ 171, ¹H NMR (400 MHz, CDCl₃, ppm) δ 7.65 (d, J 15.7 Hz, 1H), 7.13 (s, 1H), 7.11 (s, 1H), 6.24 (d, J 15.7 Hz, 1H), 4.25 (q, J 7.1 Hz, 2H), 1.32 (t, J 7.1 Hz, 3H).

### Synthesis of (E)-3-(4-chlorothiophen-2-yl)acrylic acid

Ethyl (E)-3-(4-chlorothiophen-2-yl)acrylate (2.703 g, 12.5 mmol) was dissolved in a mixture of THF (15 mL) and industrial methylated spirit (15 mL). Aqueous sodium hydroxide (2M, 17 mL, 34 mmol) was added and the mixture was stirred at room temperature for 1 h 40 min. The mixture was acidified to pH 2 using hydrochloric acid (2M). The mixture was diluted with water (40 mL) and extracted with ethyl acetate (3×50 mL). The extracts were dried (MgSO₄), filtered and concentrated in vacuo to give (E)-3-(4-chlorothiophen-2-yl)acrylic acid (2.325 g, 99%), as a pale brown solid. ESI-MS [M−CO₂H]⁻ 143, ¹H NMR (400 MHz, CDCl₃, ppm) δ 7.75 (d, J 15.7 Hz, 1H), 7.19 (s, 1H), 7.16 (s, 1H), 6.25 (d, J 15.7 Hz, 1H).

### Synthesis of (E)-3-(4-chlorothiophen-2-yl)-N-methoxy-N-methylacrylamide

(E)-3-(4-Chlorothiophen-2-yl)acrylic acid (2.325 g, 12.3 mmol), 1-ethyl-3-(3-(N,N-dimethylamino)propyl)carbodiimide (3.287 g, 17.2 mmol), triethylamine (1.90 mL, 25.9 mmol), (4-dimethylamino)pyridine (1.637 g, 13.4 mmol) and N,O-dimethylhydroxylamine hydrochloride (1.516 g, 15.5 mmol) were mixed together in DCM (40 mL) and stirred at room temperature under nitrogen for 15.5 h. The mixture was diluted with DCM (50 mL) and washed with water (2×75 mL) then brine (75 mL). The organic phase was dried (MgSO₄), filtered and concentrated in vacuo to give a red oil (4.074 g). Purification by flash column chromatography (SiliCyCle SiliaSep cartridge, 120 g), eluting with 5% ethyl acetate in heptane for 2 column volumes and then 5-50% ethyl acetate in heptane over 15 column volumes gave (E)-3-(4-chlorothiophen-2-yl)-N-methoxy-N-methylacrylamide (2.143 g, 75%) as a pale-yellow oil. ESI-MS [M+H]⁺ 232, ¹H NMR (400 MHz, CDCl₃, ppm) δ 7.70 (d, J 15.7 Hz, 1H), 7.12 (s, 1H), 7.11 (s, 1H), 6.83 (d, J 15.7 Hz, 1H), 3.75 (s, 3H), 3.29 (s, 3H).

### Synthesis of (1S*,2S*)-2-(4-chlorothiophen-2-yl)-N-methoxy-N-methylcyclopropane-1-carboxamide

Sodium hydride (60% dispersion in oil, 0.660 g, 16.3 mmol) was washed with heptane (2×15 mL) and suspended in anhydrous DMSO (10 mL). To this suspension was added a solution of trimethylsulfoxonium iodide (3.310 g, 15.0 mmol) in anhydrous DMSO (30 mL) dropwise over 15 min to give a cloudy suspension. The mixture was stirred at room temperature under nitrogen for 1 h, during which time it became a pale-yellow translucent solution. To this was added a solution of (E)-3-(4-chlorothiophen-2-yl)-N-methoxy-N-methylacrylamide (2.143 g, 9.3 mmol) in anhydrous DMSO (5 mL) dropwise over 5 min. The mixture was stirred at room temperature for 16 h, poured into water (100 mL) and extracted with ethyl acetate (5×50 mL). The combined organic extracts were washed with water (2×100 mL) and brine (100 mL). The organic solution was dried (MgSO₄), filtered and concentrated in vacuo to give a yellow oil. Purification by flash column chromatography (SiliCyCle SiliaSep cartridge, 40 g), eluting with heptane for 2 column volume followed by 5-20% ethyl acetate in heptane over 15 column volumes and then 20% ethyl acetate in heptane for 5 column volumes gave (1S*,2S*)-2-(4-chlorothiophen-2-yl)-N-methoxy-N-methylcyclopropane-1-carboxamide (1.748 g, 77%) as a yellow oil. ESI-MS [M+H]⁺ 246. ¹H NMR (400 MHz, CDCl₃, ppm) δ 6.87 (d, J 1.8 Hz, 1H), 6.69 (s, 1H), 3.74 (s, 3H), 3.24 (s, 3H), 2.63-2.58 (m, 1H), 2.43 (br s, 1H), 1.67-1.63 (m, 1H), 1.31-1.27 (m, 1H).

### Synthesis of (1S*,2S*)-2-(4-chlorothiophen-2-yl)cyclopropane-1-carboxylic acid

(1S*,2S*)-2-(4-chlorothiophen-2-yl)-N-methoxy-N-methylcyclopropane-1-carboxamide (1.748 g, 7.10 mmol) was dissolved in methanol (14 mL) and aqueous sodium hydroxide (2M, 8.0 mL, 16 mmol) was added. The mixture was stirred at 80° C. (oil bath) under nitrogen for 3.5 h. The mixture was cooled to room temperature and then acidified to pH 2 using 2M aqueous hydrochloric acid. The mixture was diluted with water (20 mL) and extracted with ethyl acetate (3×30 mL). The extracts were dried (MgSO₄), filtered and concentrated in vacuo to give a pale-yellow oil (1.851 g). Purification by flash column chromatography (SiliCyCle SiliaSep cartridge, 120 g), eluting with 5% ethyl acetate in heptane+1% acetic acid for 2 column volumes followed by 5-60% ethyl acetate in heptane+1% acetic acid over 15 column volumes) gave a pale-yellow oil, which crystallised on standing (1.338 g). Recrystallisation from hot heptane gave (1S*,2S*)-2-(4-chlorothiophen-2-yl)cyclopropane-1-carboxylic acid (0.601 g, 42%), as a colourless solid. ESI-MS [M−H]⁻ 201, ¹H NMR (400 MHz, CDCl₃, ppm) δ 6.90 (d, J 1.2 Hz, 1H), 6.71 (s, 1H), 2.70 (ddd, J 10, 6, 4 Hz, 1H), 1.93 (ddd, J 9, 6, 5 Hz, 1H), 1.68 (ddd, J 10, 7, 5 Hz, 1H), 1.38 (ddd, J 9, 7, 4 Hz, 1H).

### Synthesis of rac-(1S*,2S*)-2-(4-chlorothiophen-2-yl)cyclopropane-1-carboxamide

A mixture of rac-(1S*,2S*)-2-(4-chlorothiophen-2-yl)cyclopropane-1-carboxylic acid (300 mg, 1.49 mmol), (NH₄)₂CO₃ (713 mg, 7.43 mmol), HOBt (402 mg, 2.98 mmol), EDCI (566 mg, 2.98 mmol) and DIPEA (577 mg, 4.47 mmol) in DMF (10 mL) was stirred at room temperature for 12 h. The mixture was quenched with water (100 mL) then extracted with EtOAc (5×50 mL). The combined organic layers were washed with brine (50 mL), dried over Na₂SO₄ and concentrated in vacuo. The residue was purified by silica gel chromatography (eluant: EtOAc/PE from 0 to 30%) to afford rac-(1S*,2S*)-2-(4-chlorothiophen-2-yl)cyclopropane-1-carboxamide (250 mg, 83%) as an off-white solid. ESI-MS [M+H]⁺: 202.1.

### Synthesis of rac-tert-butyl (3-((1S*,2S*)-2-carbamoylcyclopropyl)-5-chlorophenyl)carbamate

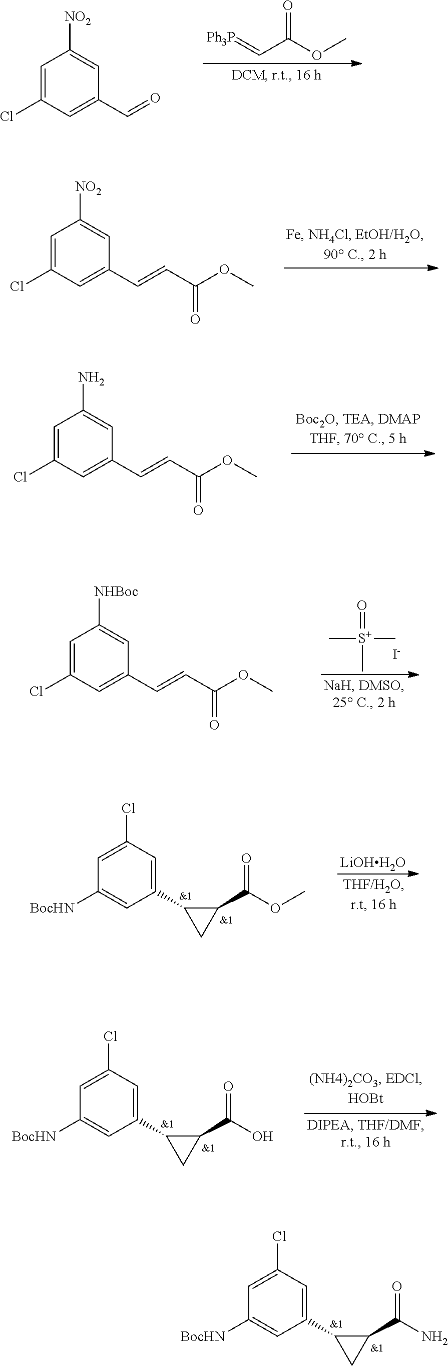

### Synthesis of methyl (E)-3-(3-chloro-5-nitrophenyl)acrylate

To a solution of 3-chloro-5-nitrobenzaldehyde (1.75 g, 9.5 mmol) in DCM (50 mL) was added methyl 2-(triphenyl-phosphanylidene)acetate (3.8 g, 11.3 mmol) and the mixture was stirred at room temperature for 16 h. The mixture was quenched with water (100 mL) and extracted with DCM (3×50 mL). The combined organic layers were washed with brine (50 mL), dried over Na₂SO₄ and concentrated in vacuo. The residue was purified by silica gel chromatography (eluent: EtOAc/PE from 0 to 15%) to give methyl (E)-3-(3-chloro-5-nitrophenyl)acrylate (2 g, 88%) as a white solid. ESI-MS [M+H]⁺: 242.1.

### Synthesis of methyl (E)-3-(3-amino-5-chlorophenyl)acrylate

A mixture of methyl (E)-3-(3-chloro-5-nitrophenyl)acrylate (2 g, 8.3 mmol), Fe (4.6 g, 83 mmol) and NH₄Cl (4.4 g, 83 mmol) in EtOH/H₂O (60 mL/20 mL) was stirred at 90° C. for 2 h. The reaction mixture was poured into water (100 mL) and filtered. The filtrate was extracted with EtOAc (3×40 ml). The combined organics were washed with brine (40 mL), dried over Na₂SO₄ and concentrated in vacuo. The residue was purified by silica gel chromatography (eluent: EtOAc/PE from 0 to 15%) to give methyl (E)-3-(3-amino-5-chlorophenyl)acrylate (1.75 g, 98%) as a yellow solid. ESI-MS [M+H]⁺: 212.1.

### Synthesis of methyl (E)-3-(3-((tert-butoxycarbonyl)amino)-5-chlorophenyl)acrylate

To a mixture of methyl (E)-3-(3-amino-5-chlorophenyl)acrylate (1.9 g, 9 mmol), Et₃N (2.73 g, 27 mmol) and DMAP (1.65 g, 13.5 mmol) in THF (50 mL) was added Boc₂O (3.93 g, 18 mmol) and the mixture was stirred at 70° C. for 5 h. The mixture was cooled to room temperature, diluted with water (100 mL) then extracted with EtOAc (3×50 mL). The combined organics were washed with brine (50 mL), dried over Na₂SO₄ and concentrated. The residue was purified by silica gel chromatography (eluent: EtOAc/PE from 0 to 15%) to give methyl (E)-3-(3-((tert-butoxycarbonyl)amino)-5-chlorophenyl)acrylate (2.3 g, 82%) as a white solid. ESI-MS [M−55]⁺: 256.1.

### Synthesis of rac-methyl (1S*,2S*)-2-(3-((tert-butoxycarbonyl)amino)-5-chlorophenyl)cyclopropane-1-carboxylate

To a stirred solution of trimethylsulfoxonium iodide (960 mg, 4.4 mmol) in dry DMSO (20 mL) was added NaH (180 mg, 4.4 mmol) at 25° C. and the mixture was stirred at 25° C. for 1 h. A solution of methyl (E)-3-(3-((tert-butoxy carbonyl)amino)-5-chlorophenyl)acrylate (1 g, 3.2 mmol) in DMSO (10 mL) was then added dropwise. The mixture was stirred at 25° C. for 2 h, quenched with water (100 mL) and extracted with EtOAc (2×100 ml). The combined organics were washed with water (3×100 mL) and brine (100 mL), dried over Na₂SO₄, concentrated in vacuo and purified by silica gel chromatography (eluent: EtOAc/PE from 0 to 15%) to give rac-methyl (1S*,2S*)-2-(3-((tert-butoxycarbonyl)amino)-5-chlorophenyl)cyclopropane-1-carboxylate (240 mg, 23%) as a white solid. ESI-MS [M+Na]⁺: 348.2.

### Synthesis of rac-tert-butyl (3-((1S*,2S*)-2-carbamoylcyclopropyl)-5-chlorophenyl)carbamate

Using a similar procedure to that for 2-(5-chloro-2-nitrophenyl)cyclopropane-1-carboxamide, rac-tert-butyl (3-((1S*,2S*)-2-carbamoylcyclopropyl)-5-chlorophenyl)carbamate (220 mg, 76%) was synthesised from rac-methyl (1S*,2S*)-2-(3-((tert-butoxycarbonyl)amino)-5-chlorophenyl)cyclopropane-1-carboxylate (240 mg, 0.74 mmol). ESI-MS [M+Na]+: 333.1.

### Synthesis of rac-(1S*,2S*)-2-(2-chloro-6-methoxypyridin-4-yl)cyclopropane-1-carboxamide

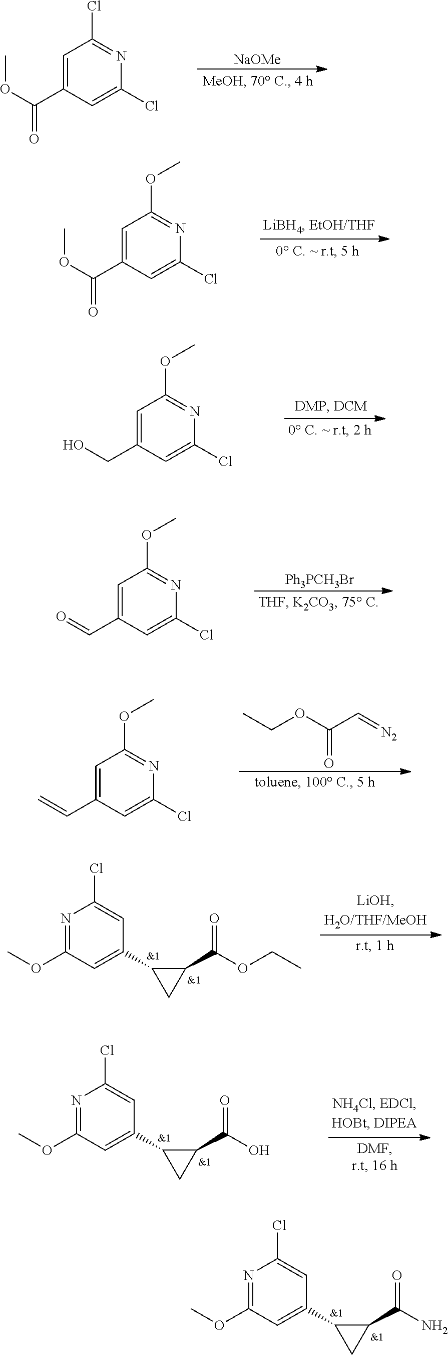

### Synthesis of methyl 2-chloro-6-methoxyisonicotinate

Na (2.23 g, 97.0 mmol) was added to a MeOH (100 mL) with stirring at 0° C. in portions and the mixture was stirred at room temperature for 1 h. Methyl 2,6-dichloroisonicotinate (10 g, 48.54 mmol) was added in portions and the resulting mixture was stirred at 70° C. for 4 h. The reaction mixture was adjusted to pH 9˜10 by NaHCO₃ aqueous (100 mL) and MeOH was removed in vacuo. The mixture was extracted with EtOAc (3×50 ml). The combined organics were washed with brine (50 mL), dried over Na₂SO₄ and concentrated in vacuo. The residue was purified by silica gel chromatography (eluent: EtOAc/PE=1/5) to give methyl 2-chloro-6-methoxyisonicotinate (7.5 g, 77%) as a white solid. ESI-MS [M+H]⁺: 202.2.

### Synthesis of (2-chloro-6-methoxypyridin-4-yl)methanol

To a stirred solution of methyl 2-chloro-6-methoxyisonicotinate (4.5 g, 22.32 mmol) in THF (60 mL) and EtOH (20 mL) was added LiBH₄ (972 mg, 44.64 mmol) in portions at 0° C. and the mixture was stirred at room temperature for 5 h. The reaction mixture was quenched with saturated NH₄Cl aqueous (100 mL) and extracted with EtOAc (3×100 ml). The combined organics were washed with brine (100 mL), dried over Na₂SO₄, concentrated in vacuo to give (2-chloro-6-methoxypyridin-4-yl)methanol (3.8 g, 98%) as a white solid which was used directly in the next step. ESI-MS [M+H]⁺: 174.1.

### Synthesis of 2-chloro-6-methoxyisonicotinaldehyde

To a stirred solution of (2-chloro-6-methoxypyridin-4-yl)methanol (2.15 g, 12.38 mmol) in DCM (80 mL) was added Dess-Martin periodinane (7.88 g, 18.58 mmol) in portions at 0° C. The mixture was stirred at room temperature for 2 h, filtered and rinsed with DCM (50 mL). The filtrate was washed with saturated NaHCO₃ aqueous (80 mL) and brine (80 mL), dried over Na₂SO₄ and concentrated in vacuo. The residue was purified by silica gel chromatography (eluent: EtOAc/PE=1/10) to give 2-chloro-6-methoxyisonicotinaldehyde (2.0 g, 94%) as a colorless syrup. ESI-MS [M+H]⁺: 172.1.

### Synthesis of 2-chloro-6-methoxy-4-vinylpyridine

To a solution of 2-chloro-6-methoxyisonicotinaldehyde (3.2 g, 18.65 mmol) in THF (50 mL) was added bromo(methyl)triphenyl-λ5-phosphane (9.99 g, 27.98 mmol) and K₂CO₃ (7.73 g, 55.95 mmol) at room temperature. The mixture was stirred at 70° C. for 16 h. The reaction mixture was poured into water (100 mL) and extracted with EtOAc (3×80 ml). The combined organics were washed with brine (80 mL), dried over Na₂SO₄ then concentrated in vacuo to give the crude, which was purified by silica gel chromatography (eluent: EtOAc/PE=1/5) to give 2-chloro-6-methoxy-4-vinylpyridine (2.2 g, 70%) as a yellow oil. ESI-MS [M+H]⁺: 170.1.

### Synthesis of rac-ethyl (1S*,2S*)-2-(2-chloro-6-methoxypyridin-4-yl)cyclopropane-1-carboxylate

To a stirred solution of 2-chloro-6-methoxy-4-vinylpyridine (2.2 g, 12.97 mmol) in toluene (40 mL) was added ethyl 2-diazoacetate (4.43 g, 38.91 mmol) in three portions over 2 h at 100° C. and the mixture was stirred at 100° C. for another 3 h. The reaction mixture was cooled, quenched with water (100 mL) and extracted with EtOAc (3×50 mL). The combined organic layers were washed with brine (50 mL), dried over Na₂SO₄ and concentrated in vacuo. The residue was purified by silica gel chromatography (EtOAc/PE from 0 to 50%) concentrated to give the crude, which was purified by silica gel chromatography (eluent: EtOAc/PE=1/5) to give rac-ethyl (1S*,2S*)-2-(2-chloro-6-methoxypyridin-4-yl)cyclopropane-1-carboxylate (1.06 g, 32%, trans-racemic) as a yellow oil. ESI-MS [M+H]⁺: 256.1.

### Synthesis of rac-(1S*,2S*)-2-(2-chloro-6-methoxypyridin-4-yl)cyclopropane-1-carboxamide

Using a similar procedure to that for rac-tert-butyl (4-((1S*,2S*)-2-carbamoylcyclopropyl)-6-methoxypyridin-2-yl)carbamate, rac-(1S*,2S*)-2-(2-chloro-6-methoxypyridin-4-yl)cyclopropane-1-carboxamide (75 mg, 77%) was synthesised from rac-ethyl (1S*,2S*)-2-(2-chloro-6-methoxypyridin-4-yl)cyclopropane-1-carboxylate (110 mg, 0.43 mmol). ESI-MS [M+H]⁺: 227.1.

### Synthesis of rac-(1S*,2S*)-2-(6-chloro-3-cyanopyridin-2-yl)cyclopropane-1-carboxamide

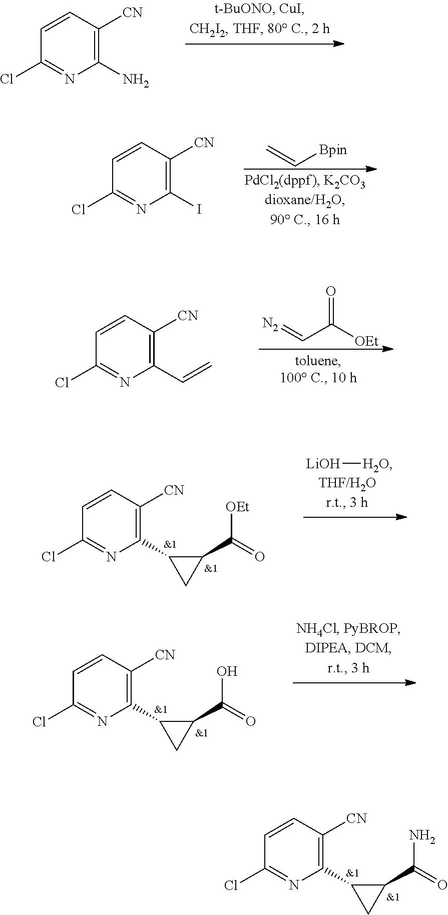

### Synthesis of 6-chloro-2-iodonicotinonitrile

A mixture of 2-amino-6-chloronicotinonitrile (857 mg, 5.6 mmol), t-BuONO (2.3 g, 22.4 mmol), CH₂I₂ (12 g, 44.8 mmol) and CuI (1.6 g, 8.4 mmol) in THF (30 mL) was stirred at 85° C. for 2 h. The reaction was concentrated in vacuo then the residue was dissolved in EtOAc (100 mL). The organics were washed with saturated Na₂S₂O₃ solution (40 mL), saturated NaHCO₃ solution (40 mL) and brine (40 mL). The organic phase was dried over Na₂SO₄, concentrated in vacuo to give the crude product, which was purified by silica gel chromatography (eluent: EtOAc/PE from 0 to 10%) to give 6-chloro-2-iodonicotinonitrile as a yellow solid. (900 mg, 61%). ESI-MS [M+H]⁺: 264.9.

### Synthesis of 6-chloro-2-vinylnicotinonitrile

To a solution of 6-chloro-2-iodonicotinonitrile (530 mg, 2 mmol) in 1,4-dioxane (12 mL) and water (3 mL) was added K₂CO₃ (828 mg, 6 mmol), Pd(dppf)Cl₂ (146 mg, 0.2 mmol) and 4,4,5,5-tetramethyl-2-vinyl-1,3,2-dioxaborolane (308 mg, 2 mmol). The resulting mixture was stirred at 90° C. overnight. After cooling to room temperature, the mixture was diluted with water (50 mL) and extracted with EtOAc (3×30 mL). The combined organic layers were washed with brine (30 mL), dried over Na₂SO₄, filtered, and concentrated in vacuo. The residue was purified by silica gel chromatography column (eluent: EtOAc/PE from 0 to 10%) to give 6-chloro-2-vinylnicotinonitrile (250 mg, 76%) as white solid. ESI-MS [M+H]⁺: 165.0.

### Synthesis of rac-ethyl (1S*,2S*)-2-(6-chloro-3-cyanopyridin-2-yl)cyclopropane-1-carboxylate

To a reaction mixture of 6-chloro-2-vinylnicotinonitrile (250 mg, 1.5 mol) in toluene (10 mL) was added ethyl 2-diazoacetate (513 mg, 4.5 mol). The reaction was stirred at 100° C. for 10 h. The reaction was cooled to room temperature and concentrated in vacuo to give the crude, which was purified by silica gel chromatography (eluent: EtOAc/PE from 0 to 10%) to afford rac-ethyl (1S*,2S*)-2-(6-chloro-3-cyanopyridin-2-yl)cyclopropane-1-carboxylate (230 mg, 60%) as white solid. ESI-MS [M+H]+: 251.0.

### Synthesis of rac-(1S*,2S*)-2-(6-chloro-3-cyanopyridin-2-yl)cyclopropane-1-carboxamide

Using a similar procedure to that for rac-(1S*,2S*)-2-(3-cyano-6-methylpyridin-2-yl)cyclopropane-1-carboxamide, rac-(1S*,2S*)-2-(6-chloro-3-cyanopyridin-2-yl)cyclopropane-1-carboxamide (120 mg, 69%) was synthesised from rac-ethyl (1S*,2S*)-2-(6-chloro-3-cyanopyridin-2-yl)cyclopropane-1-carboxylate (230 mg, 0.92 mmol). ESI-MS [M+H]+: 222.1.

### Synthesis of rac-tert-butyl (tert-butoxycarbonyl)(4-((1S*,2S*)-2-carbamoylcyclopropyl)-2-chlorophenyl)carbamate

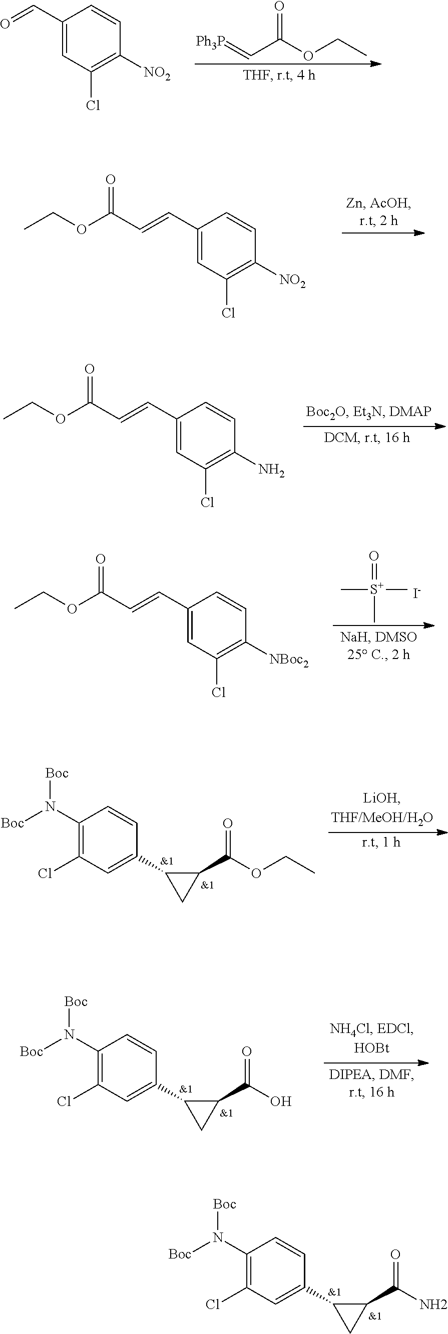

### Synthesis of ethyl (E)-3-(3-chloro-4-nitrophenyl)acrylate

To a solution of 3-chloro-4-nitrobenzaldehyde (1.5 g, 8.08 mmol) in THF (40 mL) was added ethyl (triphenylphosphoranylidene)acetate (3.66 g, 10.51 mmol) and the mixture was stirred at room temperature for 16 h. The reaction mixture was concentrated in vacuo to give the crude, which was purified by silica gel chromatography (eluent: EtOAc/PE=1/10) to give ethyl (E)-3-(3-chloro-4-nitrophenyl)acrylate (1.8 g, 87%) as a yellow solid. ESI-MS [M+H]⁺: 256.1.

### Synthesis of ethyl (E)-3-(4-amino-3-chlorophenyl)acrylate

A mixture of ethyl (E)-3-(3-chloro-4-nitrophenyl)acrylate (490 mg, 1.92 mmol) and Zn (1.26 g, 19.3 mmol) in AcOH (10 mL) was stirred at room temperature for 16 h. The reaction mixture was poured into water (30 mL), adjusted to pH 9˜10 with NaHCO₃ solution and filtered. The filtrate was extracted with EtOAc (3×50 ml). The combined organics were washed with brine (50 mL), dried over Na₂SO₄, concentrated in vacuo to give the crude, which was purified by silica gel chromatography (eluent: EtOAc/PE=1/10) to give ethyl (E)-3-(4-amino-3-chlorophenyl)acrylate (390 mg, 90%) as a yellow solid. ESI-MS [M+H]⁺: 226.1.

### Synthesis of ethyl (E)-3-(4-(di-(tert-butoxycarbonyl)amino)-3-chlorophenyl)acrylate

To a mixture of ethyl (E)-3-(4-amino-3-chlorophenyl)acrylate (390 mg, 1.73 mmol), Et₃N (875 mg, 8.65 mmol) and DMAP (106 mg, 0.865 mmol) in DCM (10 mL) was added dropwise Boc₂O (943 mg, 4.32 mmol) and the mixture was stirred at room temperature for 16 h. The reaction mixture was diluted with DCM (50 mL) and washed with water (40 mL) and brine (40 mL), dried over Na₂SO₄ then concentrated in vacuo to give the crude, which was purified by silica gel chromatography (eluent: EtOAc/PE=1/10) to give ethyl (E)-3-(4-(di-(tert-butoxycarbonyl)amino)-3-chlorophenyl)acrylate (260 mg, 35%) as a yellow solid. ESI-MS [M+Na]⁺: 448.1.

### Synthesis of rac-ethyl (1S*,2S*)-2-(4-(bis(tert-butoxycarbonyl)amino)-3-chlorophenyl)cyclopropane-1-carboxylate

To a stirred solution of trimethylsulfoxonium iodide (242 mg, 1.1 mmol) in dry DMSO (5 mL) was added NaH (44 mg, 1.1 mmol) at 25° C. After stirring the mixture for 1 h, a solution of ethyl (E)-3-(4-(di-(tert-butoxycarbonyl)amino)-3-chlorophenyl)acrylate (260 mg, 0.61 mmol) in DMSO (1 mL) was added dropwise and stirred at 25° C. for another 2 h. The reaction was quenched with water (80 mL) then extracted with EtOAc (3×30 ml). The combined organics were washed with brine (2×30 mL), dried over Na₂SO₄ then concentrated in vacuo to give the crude, which was purified by silica gel chromatography (eluent: EtOAc/PE=1/10) to give rac-ethyl (1S*,2S*)-2-(4-(bis(tert-butoxycarbonyl)amino)-3-chlorophenyl)cyclopropane-1-carboxylate (100 mg, 37%) as a white solid. ESI-MS [M+Na]⁺: 462.1.

### Synthesis of rac-tert-butyl (tert-butoxycarbonyl)(4-((1S*,2S*)-2-carbamoylcyclopropyl)-2-chlorophenyl)carbamate

Using a similar procedure to that for rac-tert-butyl (4-((1S*,2S*)-2-carbamoylcyclopropyl)-6-methoxypyridin-2-yl)carbamate, rac-tert-butyl (tert-butoxycarbonyl)(4-((1S*,2S*)-2-carbamoylcyclopropyl)-2-chlorophenyl)carbamate (40 mg, 77%) was synthesised from rac-ethyl (1S*,2S*)-2-(4-(bis(tert-butoxycarbonyl)amino)-3-chlorophenyl)cyclopropane-1-carboxylate (95 mg, 0.216 mmol). ESI-MS [M+Na]⁺: 333.1.

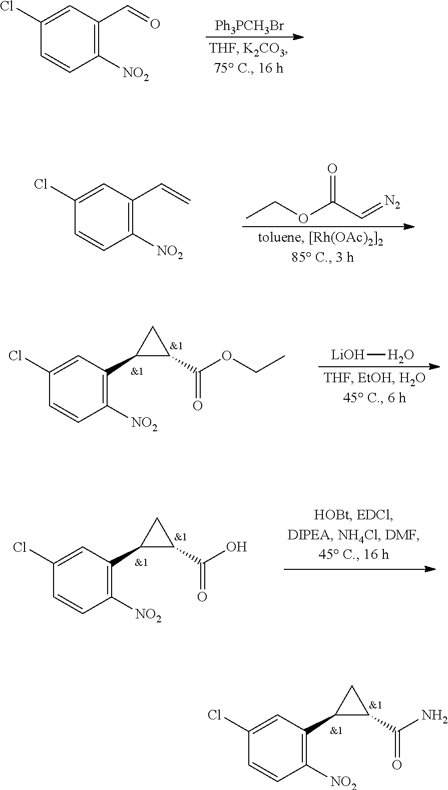

### Synthesis of rac-(1S*,2S*)-2-(5-chloro-2-nitrophenyl)cyclopropane-1-carboxamide

### Synthesis of 4-chloro-1-nitro-2-vinylbenzene

To a solution of 5-chloro-2-nitrobenzaldehyde (5.0 g, 27 mmol) in THF (100 mL), was added Ph₃PCH₃Br (11.0 g, 32 mmol) and K₂CO₃ (7.4 g, 54 mmol) at room temperature. The reaction mixture was stirred at 75° C. for 16 h. Water (200 ml) was added and the mixture was extracted with EtOAc (3×100 mL). The combined organics were washed with brine (100 ml), dried over Na₂SO₄ and concentrated in vacuo to give the crude which was purified by silica gel chromatography (eluent: EtOAc:PE=1:50) to afford 4-chloro-1-nitro-2-vinylbenzene (2.3 g, 46.5%) as a yellow oil. 1H NMR (400 MHz, DMSO) δ 8.02 (d, J=8.7 Hz, 1H), 7.90 (d, J=2.3 Hz, 1H), 7.64-7.61 (m, 1H), 7.02-6.95 (m, 1H), 6.03 (d, J=17.3 Hz, 1H), 5.58 (d, J=11.1 Hz, 1H).

### Synthesis of rac-ethyl (1S*,2S*)-2-(5-chloro-2-nitrophenyl)cyclopropane-1-carboxylate

Ethyl 2-diazoacetate (2.8 g, 24.6 mmol) was added slowly to a mixture of 4-chloro-1-nitro-2-vinylbenzene (1.5 g, 8.2 mmol and [Rh(OAc)₂]₂ (73 mg, 0.164 mmol) in toluene (30 ml) at 85° C. The reaction mixture was stirred at the same temperature for 3 h. After cooling to room temperature, the mixture was concentrated in vacuo to give the crude which was purified by silica gel chromatography (eluent: EtOAc:PE=1:40) to afford rac-ethyl (1S*,2S*)-2-(5-chloro-2-nitrophenyl)cyclopropane-1-carboxylate (700 mg, 32%) as a yellow oil.

ESI-MS: [M+H]+, 270.1

### Synthesis of rac-(1S*,2S*)-2-(5-chloro-2-nitrophenyl)cyclopropane-1-carboxamide

Using a similar procedure to that for rac-tert-butyl (4-((1S*,2S*)-2-carbamoylcyclopropyl)-6-methoxypyridin-2-yl)carbamate, rac-(1S*,2S*)-2-(5-chloro-2-nitrophenyl)cyclopropane-1-carboxamide (400 mg, 64% in 2 steps) was synthesised from rac-ethyl (1S*,2S*)-2-(5-chloro-2-nitrophenyl)cyclopropane-1-carboxylate (700 mg, 2.6 mmol). ESI-MS: [M+H]+, 241.1

### Synthesis of rac-methyl (1S*,2R*)-5′-chloro-2′-oxospiro[cyclopropane-1,3′-indoline]-2-carboxylate and rac-methyl (1S*,2S*)-5′-chloro-2′-oxospiro[cyclopropane-1,3′-indoline]-2-carboxylate

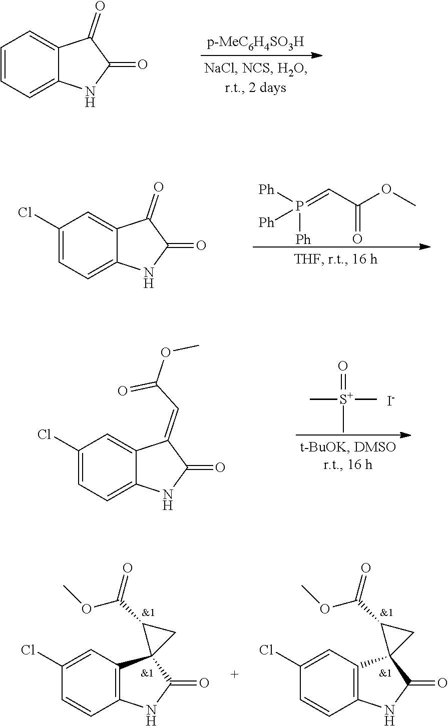

### Synthesis of 5-chloroindoline-2,3-dione

A mixture of indoline-2,3-dione (3.5 g, 24 mmol), p-MeC₆H₄SO₃H (6.8 g, 36 mmol), NaCl (2.8 g, 48 mmol) and NCS (4.8 g, 36 mmol) in water (50 mL) was stirred at room temperature for 2 days. The precipitates were collected by filtration and dried in an oven to give 5-chloroindoline-2,3-dione (4 g, 93%) as an orange solid. ESI-MS [M+H]+: 182.1.

### Synthesis of methyl (E)-2-(5-chloro-2-oxoindolin-3-ylidene)acetate

To a solution of 5-chloroindoline-2,3-dione (4 g, 22 mmol) in THF (20 mL) was added methyl 2-(triphenyl-15-phosphanylidene)acetate (8 g, 24 mmol). The reaction mixture was stirred at room temperature for 16 h. then concentrated in vacuo. The resulting residue purified by silica gel chromatography (eluent: PE/EtOAc=2/1) to give methyl (E)-2-(5-chloro-2-oxoindolin-3-ylidene)acetate (1.7 g, 33%) as an orange solid. ESI-MS: [M+H]⁺: 238.1.

### Synthesis of rac-methyl (1S*,2R*)-5′-chloro-2′-oxospiro[cyclopropane-1,3′-indoline]-2-carboxylate and rac-methyl (1S*,2S*)-5′-chloro-2′-oxospiro[cyclopropane-1,3′-indoline]-2-carboxylate

A mixture of trimethylsulfoxonium iodide (2.3 g, 10.5 mmol) and t-BuOK (10.5 mL, 10.5 mmol, 1M in THF) in DMSO (5 mL) was stirred at room temperature for 1 h under N₂. Then a solution of methyl (E)-2-(5-chloro-2-oxoindolin-3-ylidene)acetate (1.7 g, 7 mmol) in DMSO/THF (5 mL/10 mL) was added. The reaction mixture was stirred at room temperature for 16 h then acidified to pH 7 with HCl (1 M, aq·). The mixture was extracted with EtOAc (3×50 mL). The combined organic layers were washed with brine (2×20 mL), dried over Na₂SO₄, concentrated in vacuo to give the crude, which was purified by column chromatography (eluent: DCM/MeOH=20/1) to give rac-methyl (1S*,2R*)-5′-chloro-2′-oxospiro[cyclopropane-1,3′-indoline]-2-carboxylate (isomer 1: 250 mg, 14%) and rac-methyl (1S*,2S*)-5′-chloro-2′-oxospiro[cyclopropane-1,3′-indoline]-2-carboxylate (isomer 2: 300 mg, 17%) as yellow solids. ESI-MS [M+H]+: 252.0.

### Synthesis of rac-(1S*,2S*)-2-(3-chloro-2-nitrophenyl)cyclopropane-1-carboxamide

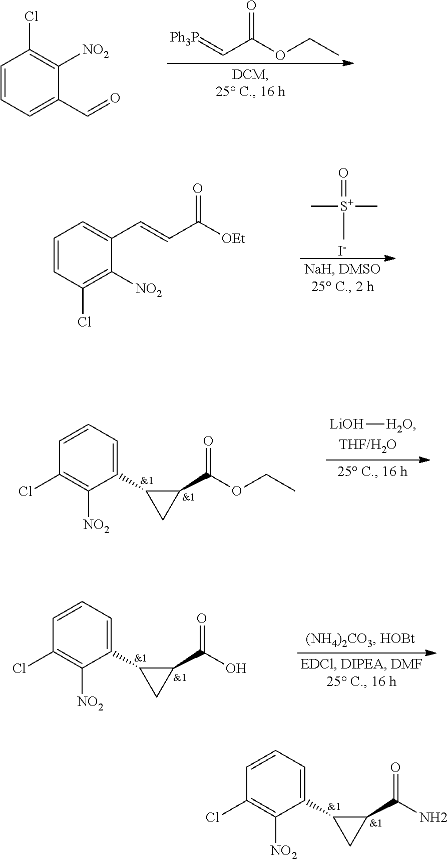

### Synthesis of ethyl (E)-3-(3-chloro-2-nitrophenyl)acrylate

A mixture of 3-chloro-2-nitrobenzaldehyde (2 g, 10.7 mmol) and ethyl 2-(triphenyl-15-phosphanylidene)acetate (4.5 g, 12.9 mmol) in DCM (80 mL) was stirred at 25° C. for 16 h under N₂. The mixture concentrated to give a crude product which was purified by flash column chromatography (eluent: PE:EtOAc=0 to 100%) to give ethyl (E)-3-(3-chloro-2-nitrophenyl)acrylate (1.5 g, 55% yield) as an off-white solid. ESI-MS [M+H]+: 256.0

### Synthesis of rac-ethyl (1S*,2S*)-2-(3-chloro-2-nitrophenyl)cyclopropane-1-carboxylate

To a mixture of NaH (410 mg, 10 mmol) in DMSO (10 mL) was added trimethylsulfoxonium iodide (2.01 g, 9 mmol) slowly at 25° C. under N₂. The reaction mixture was then stirred at 25° C. for 0.5 h until a clear solution was obtained. Ethyl (E)-3-(3-chloro-2-nitrophenyl)acrylate (1.3 g, 5 mmol) in DMSO (5 mL) was added and the mixture was stirred at 25° C. for 2 h. The mixture was quenched with saturated NH₄Cl solution (150 mL) and extracted with EtOAc (3×50 mL). The organic layers were washed with water (30 mL), brine (30 mL), dried over Na₂SO₄ and concentrated in vacuo. The crude product was purified by silica gel column chromatography (eluent: MeOH/DCM from 0 to 10%) to give rac-ethyl (1S*,2S*)-2-(3-chloro-2-nitrophenyl)cyclopropane-1-carboxylate (300 mg, 22% yield) as a yellow oil. ESI-MS [M+H]+: 270.1

### Synthesis of rac-(1S*,2S*)-2-(3-chloro-2-nitrophenyl)cyclopropane-1-carboxamide

Using a similar procedure to that for 2-(5-chloro-2-nitrophenyl)cyclopropane-1-carboxamide, rac-(1S*,2S*)-2-(3-chloro-2-nitrophenyl)cyclopropane-1-carboxamide (225 mg, 85% yield) was synthesised from rac-ethyl (1S*,2S*)-2-(3-chloro-2-nitrophenyl)cyclopropane-1-carboxylate (300 mg, 1.11 mmol). ESI-MS [M+H]+: 241.1

### Synthesis of rac-(1S*,2S*)-2-(2-cyano-5-methoxyphenyl)cyclopropane-1-carboxamide

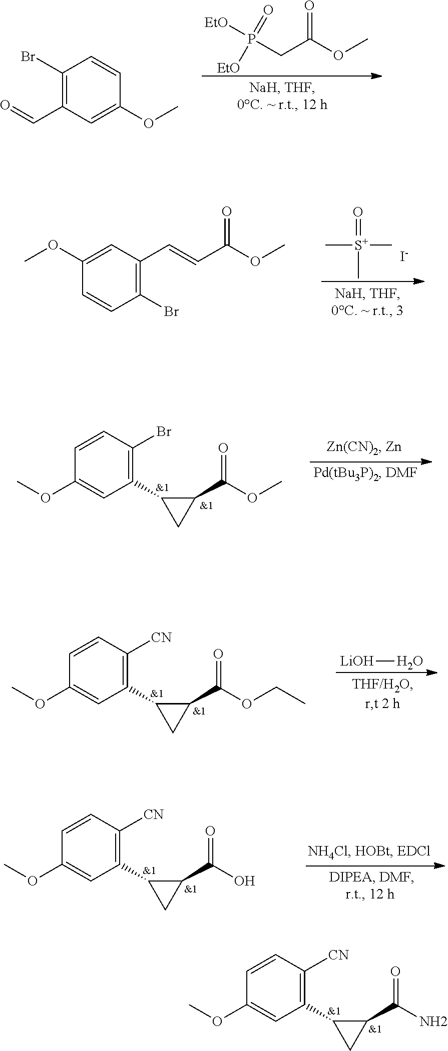

### Synthesis of methyl (E)-3-(2-bromo-5-methoxyphenyl)acrylate

To a solution of methyl 2-(diethoxyphosphoryl)acetate (1.1 mL, 5.75 mmol) in THF (10 mL) was added NaH (220 mg, 5.5 mmol) at 0° C. The mixture was stirred for 5 min then 2-bromo-5-methoxybenzaldehyde (1.07 g, 5.0 mmol) in THF (10 mL) was added dropwise. The mixture was stirred at room temperature for 12 h. Water (50 mL) was added and the mixture extracted with EtOAc (3×40 mL). The combined organic layers were washed with brine (40 mL), dried over Na₂SO₄ and concentrated in vacuo to give the crude, which was purified by silica gel chromatography (eluent: PE/EtOAc=10/1) to give methyl (E)-3-(2-bromo-5-methoxyphenyl)acrylate (1.2 g, 89%) as a white solid. ESI-MS [M+H]+: 271.1.

### Synthesis of rac-methyl (1S*,2S*)-2-(2-bromo-5-methoxyphenyl)cyclopropane-1-carboxylate

NaH (320 mg, 13.3 mmol) was added to a solution of trimethylsulfoxonium iodide (2.93 g, 13.3 mmol) in DMSO (50 mL). The mixture was stirred at room temperature for 2 h. Methyl (E)-3-(2-bromo-5-methoxyphenyl)acrylate (2.0 g, 7.4 mmol) in DMSO (30 mL) was added to the mixture and stirring was continued at room temperature for 2 h. Water (300 mL) was added and the mixture was extracted with EtOAc (3×100 mL). The combined organic layers were washed with brine (3×50 mL), dried over Na₂SO₄ and concentrated to give the crude, which was purified by silica gel chromatography (eluent: PE/EtOAc=30/1) to give rac-methyl (1S*,2S*)-2-(2-bromo-5-methoxyphenyl)cyclopropane-1-carboxylate (1.1 g, 52%) as a yellow solid. ESI-MS [M+H]+: 285.2.

### Synthesis of rac-methyl (1S*,2S*)-2-(2-cyano-5-methoxyphenyl)cyclopropane-1-carboxylate

To a mixture of rac-methyl (1S*,2S*)-2-(2-bromo-5-methoxyphenyl)cyclopropane-1-carboxylate (330 mg, 1.11 mmol), Zn(CN)₂ (129.8 mg, 1.11 mmol) and Zn (7.2 mg, 0.111 mmol) in DMF (5 mL) was added Pd(t-BuP)₂ (56.7 mg, 0.111 mmol). The resulting reaction mixture was stirred at 80° C. for 4h under N₂ then cooled to room temperature. Water (60 mL) was added and the mixture was extracted with EtOAc (3×30 mL). The combined organic layers were washed with brine (40 mL), dried over Na2SO4 and concentrated in vacuo to give the crude, which was purified by silica gel chromatography (eluent: DCM/MeOH=50/1) to give rac-methyl (1S*,2S*)-2-(2-cyano-5-methoxyphenyl)cyclopropane-1-carboxylate (125 mg, 51%) as a yellow solid. ESI-MS [M+H]+: 232.2.

### Synthesis of rac-(1S*,2S*)-2-(2-cyano-5-methoxyphenyl)cyclopropane-1-carboxamide

Using a similar procedure to that for rac-tert-butyl (4-((1S*,2S*)-2-carbamoylcyclopropyl)-6-methoxypyridin-2-yl)carbamate, rac-(1S*,2S*)-2-(2-cyano-5-methoxyphenyl)cyclopropane-1-carboxamide (300 mg, 85.7%) was synthesised from rac-methyl (1S*,2S*)-2-(2-cyano-5-methoxyphenyl)cyclopropane-1-carboxylate (500 mg, 2.16 mmol). ESI-MS [M+H]+: 217.2.

### Synthesis of rac-(1S*,2S*)-2-(5-chloro-1H-indazol-3-yl)cyclopropane-1-carboxamide

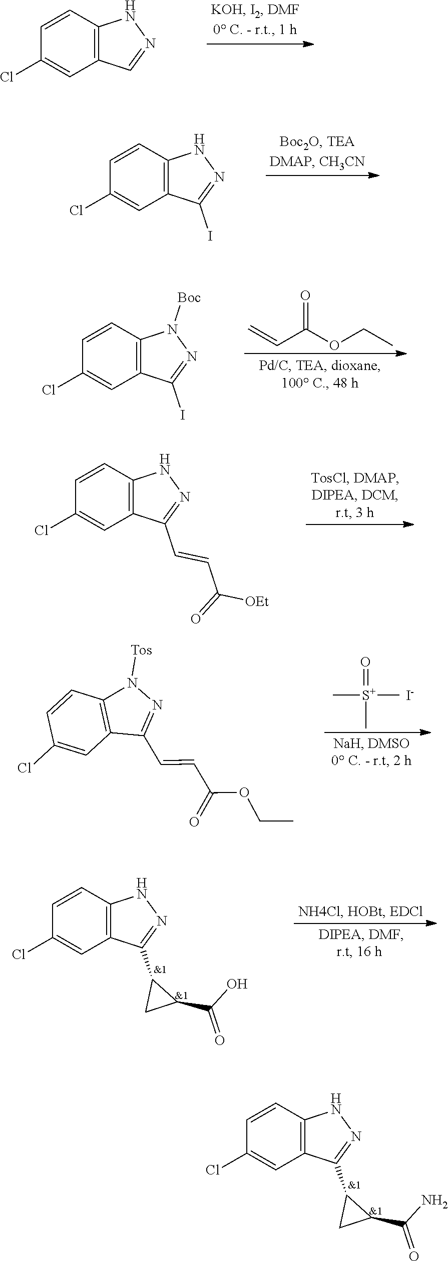

### Synthesis of 5-chloro-3-iodo-1H-indazole

To a mixture of 5-chloro-1H-indazole (5.2 g, 34 mmol) in dry DMF (50 mL) was added KOH (7.6 g, 136 mmol) and I₂ (17.2 g, 68 mmol) at 0° C. under N₂. The mixture was stirred for 1 h at 0° C. and then at room temperature for another 1 h. The reaction was quenched with water (500 mL) and extracted with EtOAc (3×300 mL). The organic layers were washed with brine (200 ml), dried over Na₂SO₄ and concentrated in vacuo to give crude 5-chloro-3-iodo-1H-indazole (9.0 g, crude) which used in the next step without further purification. ESI-MS [M+H]⁺: 279.9.

### Synthesis of 6-(chloromethyl)isoquinolin-1-amine

To a mixture of 5-chloro-3-iodo-1H-indazole (6.4 g, crude) in dry MeCN (80 mL) was added TEA (4.6 g, 46 mmol), DMAP (280 mg, 2.3 mmol) and Boc₂O (5.9 g, 27.6 mmol). The reaction was stirred at room temperature for 16 h under N₂ then poured into water (200 mL) and extracted with EtOAc (3×100 mL). The combined organics were washed with brine (100 mL), dried over Na₂SO₄ and concentrated in vacuo. The residue was purified by silica gel column (eluent: EtOAc/PE=10%) to give tert-butyl 5-chloro-3-iodo-1H-indazole-1-carboxylate (8.2 g, 94%) as white solid. ESI-MS [M−55]+: 322.9.

### Synthesis of tert-butyl 5-chloro-3-iodo-1H-indazole-1-carboxylate

To a solution of tert-butyl 5-chloro-3-iodo-1H-indazole-1-carboxylate (5 g, 13.2 mmol) in dry 1,4-dioxane (250 mL) were added ethyl acrylate (26 g, 264 mmol), Pd/C (2.75 g, 2.6 mmol) and TEA (26 g, 264 mmol). The mixture was stirred at 100° C. for 48 h under N₂, cooled to room temperature, filtered with through Celite® and washed with EtOAc (2×50 mL). The organic layers were concentrated and purified by chromatography (eluent: EtOAc/PE=15%) to give ethyl (E)-3-(5-chloro-1H-indazol-3-yl)acrylate (2 g, 61%) as a yellow solid. ESI-MS [M+H]⁺: 251.1.

### Synthesis of 6-(aminomethyl)isoquinolin-1-amine

To a solution of ethyl (E)-3-(5-chloro-1H-indazol-3-yl)acrylate (2.2 g, 8.8 mmol) in dry DCM (100 mL) was added TsCl (2.5 g, 13.2 mmol), DMAP (322 mg, 2.6 mmol) and DIPEA (3.4 g, 26.4 mmol). After stirring for 3 h under N₂, the reaction mixture was poured into water (100 mL) and extracted with DCM (3×100 ml). The combined organics were washed with brine (100 mL), dried over Na₂SO₄ and concentrated in vacuo. The residue was purified by silica gel chromatography (eluent: EtOAc/PE=10%) to give ethyl (E)-3-(5-chloro-1-tosyl-1H-indazol-3-yl)acrylate (1.7 g, 48%) as a white solid. ESI-MS [M+H]⁺: 405.0.

### Synthesis of rac-(1S*,2S*)-2-(5-chloro-1H-indazol-3-yl)cyclopropane-1-carboxylic acid

To a solution of NaH (504 mg, 12.6 mmol) in dry DMSO (20 mL) was added trimethylsulfoxonium iodide (2.7 g, 12.6 mmol) in DMSO (20 mL). The mixture was stirred at 0° C. for 0.5 h then ethyl (E)-3-(5-chloro-1-tosyl-1H-indazol-3-yl)acrylate (1.7 g, 4.2 mmol) in dry DMSO (10 mL) was added. After stirring at room temperature for 2 h under N₂, the mixture was poured into water (200 mL) and extracted with EtOAc (2×100 mL). The water layers were adjusted pH to 5 with HCl (5 ml, 1M, aq·), then extracted with EtOAc (2×100 mL). The combined organic layers were dried over Na₂SO₄ and concentrated in vacuo and purified by column (DCM/MeOH=10/1) to give rac-(1S*,2S*)-2-(5-chloro-1H-indazol-3-yl)cyclopropane-1-carboxylic acid (500 mg, 50%) as a white solid. ESI-MS [M+H]+: 237.1.

### Synthesis of rac-(1S*,2S*)-2-(5-chloro-1H-indazol-3-yl)cyclopropane-1-carboxamide

To a solution of rac-(1S*,2S*)-2-(5-chloro-1H-indazol-3-yl)cyclopropane-1-carboxylic acid (500 mg, 2.1 mmol) in dry DMF (10 mL) was added NH₄Cl (1.1 g, 21 mmol), HOBt (368 mg, 2.7 mmol), EDCI (515 mg, 2.7 mmol) and DIPEA (812 mg, 6.3 mmol). The reaction was stirred at room temperature for 16 h under N₂. Water (100 ml) was added and extracted with EtOAc (3×50 mL). The organic layers were washed with brine (50 mL), dried over Na₂SO₄ and concentrated in vacuo. The residue was concentrated and purified by silica gel chromatography (eluent: DCM/MeOH=10:1) to give rac-(1S*,2S*)-2-(5-chloro-1H-indazol-3-yl)cyclopropane-1-carboxamide (200 mg, 40%) as a white solid. ESI-MS [M+H]⁺: 236.1.

### Synthesis of ethyl (1R*,2R*,3S*)-2-(3-chlorophenyl)-3-methylcyclopropane-1-carboxylate

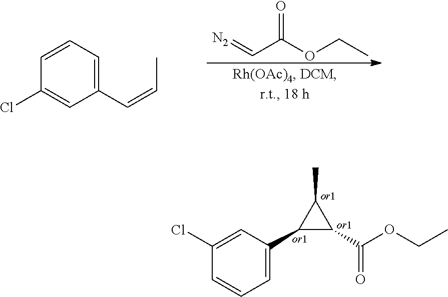

To a mixture of (Z)-1-chloro-3-(prop-1-en-1-yl)benzene (1.52 g, 10.0 mmol) and Rh(OAc)₄ (221 mg, 0.5 mmol) in DCM (30 mL) was added ethyl 2-diazoacetate (2.28 g, 20.0 mmol) dropwise. After stirring at room temperature for 18 h, the reaction mixture was diluted with water (100 mL) and extracted with DCM (3×50 mL). The combined organic layers were washed with brine (50 mL), dried over Na₂SO₄ and concentrated in vacuo. The residue was purified by silica gel chromatography (eluent: DCM/PE from 0 to 100%) to afford ethyl (1R*,2R*,3S*)-2-(3-chlorophenyl)-3-methylcyclopropane-1-carboxylate (isomer 1, 650 mg, 25%; isomer 2, 500 mg, 21%; isomer 3, 400 mg, 17%; isomer 4, 80 mg, 3.4%) as light-yellow oils.

### Synthesis of rac-(S*2S*)-2-(5-chloropyridin-3-yl)cyclopropane-1-carboxamide

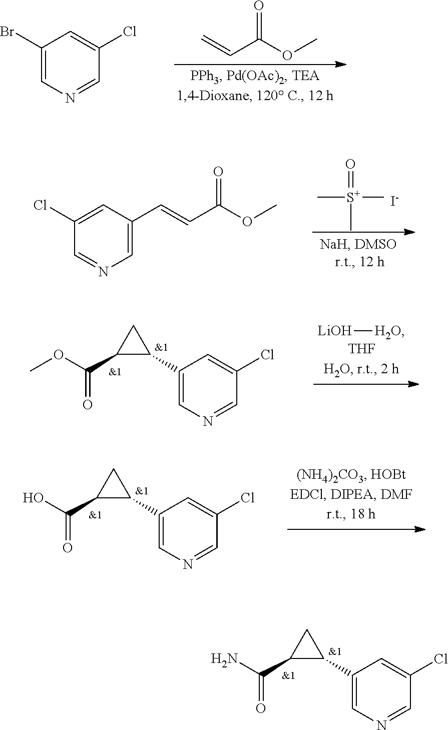

### Synthesis of methyl (E)-3-(5-chloropyridin-3-yl)acrylate

A mixture of 3-bromo-5-chloropyridine (2 g, 10.4 mmol), methyl acrylate (4.7 g, 52.0 mmol), PPh₃ (545 mg, 2.08 mmol), Pd(OAc)₂ (466 mg, 2.08 mmol), TEA (5.25 g, 52.0 mmol) and 1,4-dioxane (20 mL) was stirred at 120° C. or 12 h. The reaction was diluted with water (30 mL) then extracted with EtOAc (3×40 mL). The combined organic layers were washed with brine, dried over Na₂SO₄, filtered and concentrated in vacuo to give the crude, which was purified by silica gel chromatography (eluent: DCM\MeOH=100\1) to give methyl (E)-3-(5-chloropyridin-3-yl)acrylate (500 mg, 25%) as a white solid. ESI-MS [M+H]+: 198.2.

### Synthesis of rac-methyl (1S*,2S*)-2-(5-chloropyridin-3-yl)cyclopropane-1-carboxylate

To a solution of trimethylsulfoxonium iodide (600 mg, 91.3 mmol) in DMSO (20 mL) was added NaH (220 mg, 60% dispersion in mineral oil, 91.3 mmol. The reaction mixture was stirred at room temperature for 1 h and then a solution of methyl (E)-3-(5-chloropyridin-3-yl)acrylate (600 mg, 30.3 mmol) in DMSO (30 mL) was added. The reaction mixture was stirred at room temperature for 12 h. The reaction was quenched with water (60 mL) and extracted with EtOAc (3×60 mL). The combined organic layers were washed with brine, dried over Na₂SO₄, filtered and concentrated in vacuo to give the crude, which was purified by silica gel chromatography (eluent: PE\EA=4\1) to give rac-methyl (1S*,2S*)-2-(5-chloropyridin-3-yl)cyclopropane-1-carboxylate (220 mg, 34%) as a white solid. ESI-MS [M+H]+: 212.1

### Synthesis of rac-(1S*,2S*)-2-(5-chloropyridin-3-yl)cyclopropane-1-carboxamide

Using a similar procedure to that for 2-(5-chloro-2-nitrophenyl)cyclopropane-1-carboxamide, rac-(1S*,2S*)-2-(5-chloropyridin-3-yl)cyclopropane-1-carboxamide (130 mg, 65%) was synthesised from rac-methyl (1S*,2S*)-2-(5-chloropyridin-3-yl)cyclopropane-1-carboxylate (220 mg, 1.11 mmol). ESI-MS [M+H]+: 197.1.

### Synthesis of rac-(1S*,2S*)-2-(2-chloropyridin-4-yl)cyclopropane-1-carboxamide

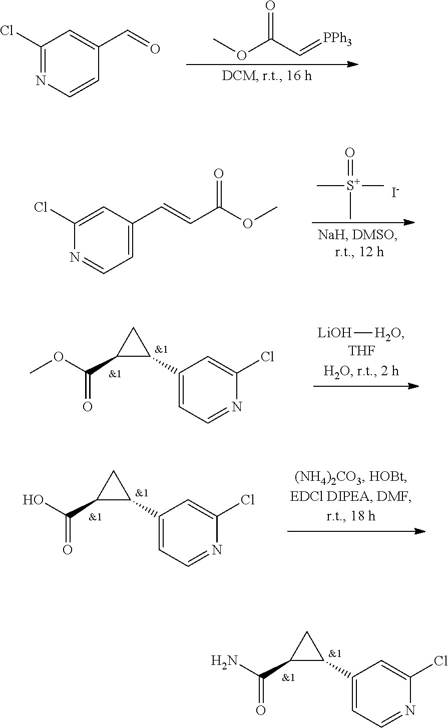

### Synthesis of methyl (E)-3-(2-chloropyridin-4-yl)acrylate

A mixture of 2-chloroisonicotinaldehyde (2 g, 14.0 mmol), methyl 2-(triphenyl-15-phosphanylidene)acetate (5.67 g, 16.96 mmol) and DCM (40 mL) was stirred at room temperature for 16 h. The reaction was concentrated in vacuo to give the crude, which was purified by silica gel chromatography (eluent: PE\EtOAc=10\1) to give methyl (E)-3-(2-chloropyridin-4-yl)acrylate (1.8 g, 64%) as a White solid. ESI-MS [M+H]+: 198.2.

### Synthesis of rac-methyl (1S*,2S*)-2-(2-chloropyridin-4-yl)cyclopropane-1-carboxylate

To a solution of Trimethylsulfoxonium iodide (600 mg, 91.3 mmol) in DMSO (20 mL) was added NaH (220 mg, 60% dispersion in mineral oil, 91.3 mmol). After stirring the resulting mixture at room temperature for 1 h, a solution of methyl (E)-3-(5-chloropyridin-3-yl)acrylate (600 mg, 30.3 mmol) in DMSO (30 mL) was added and then stirred at room temperature for another 12 h. The reaction was quenched with water (60 mL), extracted with EtOAc (3×60 mL). The combined organic layers were washed with brine, dried over Na₂SO₄, filtered and concentrated to give the crude, which was purified by silica gel chromatography (eluent: PE/EtOAc=5/1) to rac-methyl (1S*,2S*)-2-(2-chloropyridin-4-yl)cyclopropane-1-carboxylate (220 mg, 34.3%) as a white solid. ESI-MS [M+H]+: 212.1

### Synthesis of rac-(1S*,2S*)-2-(2-chloropyridin-4-yl)cyclopropane-1-carboxylic acid

A mixture of rac-methyl (1S*,2S*)-2-(2-chloropyridin-4-yl)cyclopropane-1-carboxylate (220 mg, 1.11 mmol), LiOH—H₂O (137 mg, 3.33 mmol) in THF/H₂O (2 mL/5 mL) was stirred at room temperature for 2 h. The reaction was concentrated, then the pH of the residue was adjusted to ˜4 with HCl (1.0 N, aq·) and extracted with EA (3×30 mL). The combined organic layers were washed with brine, dried over Na₂SO₄, filtered and concentrated to give rac-(1S*,2S*)-2-(2-chloropyridin-4-yl)cyclopropane-1-carboxylic acid (200 mg, crude) as a white solid, which was used into next step without further purification. ESI-MS [M+H]+: 198.2.

### Synthesis of rac-(1S*,2S*)-2-(2-chloropyridin-4-yl)cyclopropane-1-carboxamide

A mixture of rac-(1S*,2S*)-2-(2-chloropyridin-4-yl)cyclopropane-1-carboxylic acid (200 mg, crude), (NH₄)₂CO₃ (195 mg, 2.03 mmol), HOBt (205.5 mg, 1.52 mmol), EDCI (293.3 mg, 1.52 mmol), DIPEA (134.8 mg, 1.045 mmol) and DMF (3 mL) was stirred at room temperature for 12 h. The reaction was washed with water (20 mL), extracted with EtOAc (3×30 mL). The combined organic layers were washed with brine, dried over Na₂SO₄, filtered and concentrated to give the crude, which was purified by silica gel chromatography (eluent: DCM/MeOH=20/1) to give rac-(1S*,2S*)-2-(2-chloropyridin-4-yl)cyclopropane-1-carboxamide (130 mg, 65%) as a white solid. ESI-MS [M+H]+: 197.1.

### Synthesis of rac-ethyl (1S*,2S*)-2-(5-methyl-1,2,4-thiadiazol-3-yl)cyclopropane-1-carboxylate

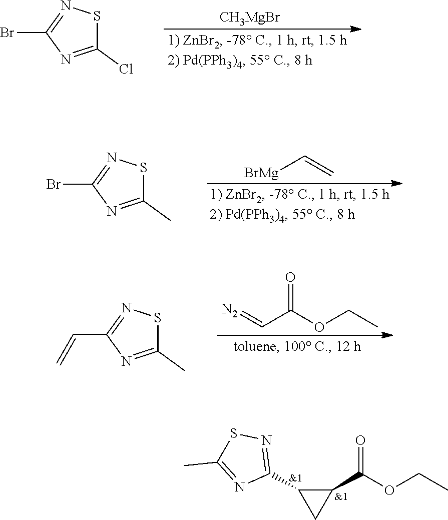

### Synthesis of 3-bromo-5-methyl-1,2,4-thiadiazole

To a mixture of ZnBr₂ (3.26 g, 14 mmoL) in THF (60 mL) and was added CH₃MgBr (4.7 mL, 3M solution in THF, 14 mmol) at −78° C. for 1 h. The resulting mixture was stirred at room temperature for 1 h and then were added 3-bromo-5-chloro-1,2,4-thiadiazole (2 g, 10 mmol) and Pd(PPh₃)₄ (1.1 g, 1 mmol). After stirring at 55° C. for 8h, the reaction was washed with water (50 mL) and extracted with EtOAc (3×50 mL). The combined organic layers were washed with brine (50 mL), dried over Na₂SO₄, concentrated in vacuo to give the crude, which was purified with silica gel column chromatography (eluent: PE/EtOAc=5/1) to give 3-bromo-5-methyl-1,2,4-thiadiazole (550 mg, 31%) as colorless oil. ESI-MS [M+H]+: 199.0.

### Synthesis of 5-methyl-3-vinyl-1,2,4-thiadiazole

To a mixture of ZnBr₂ (250 mg, 1.96 mmol) in THF (30 mL) and was added CH₃MgBr (0.65 mL, 1.96 mmol, 3M solution in THF) at −78° C. for 1 h. The resulting mixture was stirred at room temperature for 1 h and then 3-bromo-5-methyl-1,2,4-thiadiazole (250 mg, 1.6 mmol) and Pd(PPh₃)₄ (185 mg, 0.16 mmol) were added. After stirring at 55° C. for 8h, the reaction was washed with saturated aqueous NH₄Cl (30 mL), extracted with EtOAc (3×30 mL). The combined organic layers were washed with brine (30 mL), dried over Na₂SO₄ then concentrated in vacuo to give the crude, which was purified preparative TLC (eluent: PE/EtOAc=5/1) to give 5-methyl-3-vinyl-1,2,4-thiadiazole (120 mg, 60%) as a yellow oil. ESI-MS [M+H]+: 127.1.

### Synthesis of rac-ethyl (1S*,2S*)-2-(5-methyl-1,2,4-thiadiazol-3-yl)cyclopropane-1-carboxylate

To a solution of 5-methyl-3-vinyl-1,2,4-thiadiazole (120 mg, 0.95 mmol) in toluene (5 mL) was added ethyl 2-diazoacetate (760 mg, 6.66 mmol). The resulting reaction mixture was stirred at 100° C. for 12 h. The reaction was cooled to room temperature and then concentrated in vacuo to give the crude, which was purified by silica gel chromatography (eluent: PE/EtOAc=5/1) to give rac-ethyl (1S*,2S*)-2-(5-methyl-1,2,4-thiadiazol-3-yl)cyclopropane-1-carboxylate (60 mg, 30%) as a yellow oil. ESI-MS [M+H]+: 213.2.

### Synthesis of rac-ethyl (1S*,2S*)-1-fluoro-2-(4-methylpyrimidin-2-yl)cyclopropane-1-carboxylate

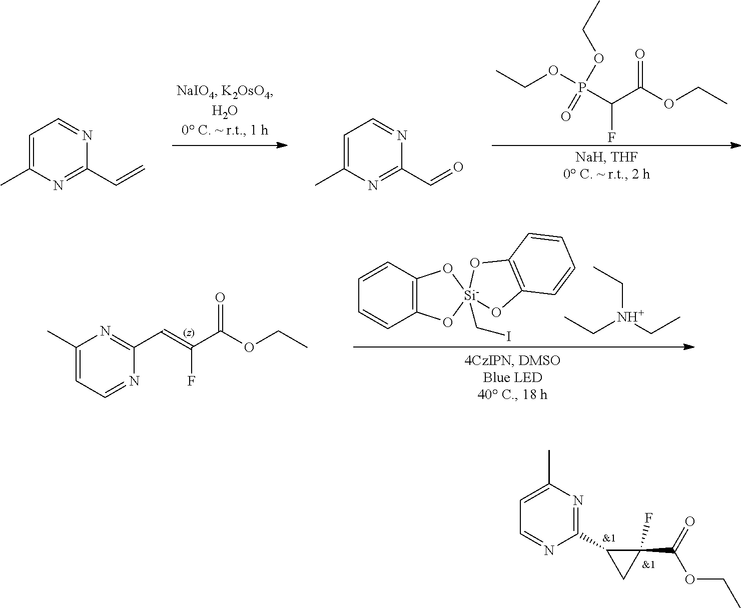

### Synthesis of 4-methylpyrimidine-2-carbaldehyde

A mixture of 4-methyl-2-vinylpyrimidine (2 g, 16.7 mmol), NaIO₄ (14.2 g, 66.8 mmol) and K₂OsO₄ (564.4 mg, 1.7 mmol) in THF (80 mL) and water (20 mL) was stirred at room temperature for 1 h. The reaction was diluted water (60 mL), extracted with EtOAc (3×60 mL). The combined organic layers were washed with brine (60 mL), dried over Na₂SO₄, filtered and concentrated in vacuo to give the crude, which was purified silica gel column chromatography (eluent: PE/EA=20/1-10/1) to give 4-methylpyrimidine-2-carbaldehyde (300 mg, 15%) as a yellow oil. ESI-MS [M+H]+: 123.2.

### Synthesis of ethyl (Z)-2-fluoro-3-(4-methylpyrimidin-2-yl)acrylate

To a solution of ethyl 2-(diethoxyphosphoryl)-2-fluoroacetate (895 mg, 3.7 mmol) in THF (30 mL) was added NaH (180 mg, 4.5 mmol, 60% dispersion in mineral oil) at 0° C. The reaction mixture was stirred for 30 min and then a solution of 4-methylpyrimidine-2-carbaldehyde (300 mg, 2.5 mmol) in THF (5 mL) was added. After stirring at room temperature for another 2 h, the reaction was quenched with saturated aqueous (50 mL) and extracted with EtOAc (3×50 mL). The combined organic layers were washed with brine (50 mL), dried over Na₂SO₄ and concentrated in vacuo to give the crude, which was purified with silica gel column chromatography (eluent: PE/EA=10/1-5/1) to give ethyl (Z)-2-fluoro-3-(4-methylpyrimidin-2-yl)acrylate (315 mg, 59%) as yellow oil. ESI-MS [M+H]+: 211.2.

### Synthesis of rac-ethyl (1S*,2S*)-1-fluoro-2-(4-methylpyrimidin-2-yl)cyclopropane-1-carboxylate

To a mixture of ethyl (Z)-2-fluoro-3-(4-methylpyrimidin-2-yl)acrylate (300 mg, 1.43 mmol) in DMSO (5 mL) were added triethylammonium bis(catecholato)iodomethylsilicate (1 g, 2.14 mmol) and 4CzIPN (54 mg, 0.07 mmol). The resulting solution was degassed with N₂ and placed in front of two blue LEDs (Kessil, H150, 32W). The reaction was stirred for 18 h. The reaction was diluted with water (30 mL) and extracted with EtOAc (3×50 mL). The combined organic layers were washed with brine (50 mL), dried over Na₂SO₄ then concentrated in vacuo to give the crude, which was purified with preparative TLC (PE/EA=2/1) to give rac-ethyl (1S*,2S*)-1-fluoro-2-(4-methylpyrimidin-2-yl)cyclopropane-1-carboxylate as yellow oil. ESI-MS [M+H]+: 224.2.

### Synthesis of (1S,2S)-2-(3-((trimethylsilyl)ethynyl)phenyl)cyclopropane-1-carboxamide

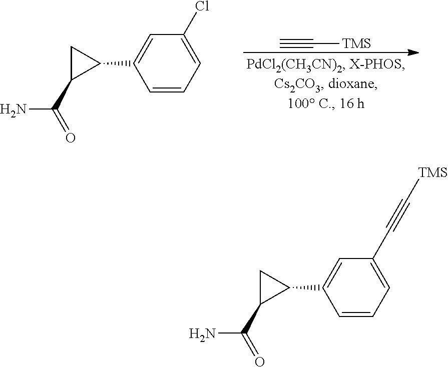

To a solution of (1S,2S)-2-(3-chlorophenyl)cyclopropane-1-carboxamide (255 mg, 1.30 mmol) in 1,4-Dioxane (5 mL) were added ethynyltrimethylsilane (384 mg, 3.9 mmol), X-PHOS (31 mg, 0.06 mmol), Cs₂CO₃ (937 mg, 2.86 mmol) and PdCl₂(CH₃CN)₂ (17 mg, 0.06 mmol). After stirring at 100° C. for 16 h, the reaction was cooled to room temperature, diluted with water (50 mL) then extracted with EtOAc (3×50 mL). The combined organic layers were washed with brine (40 mL), dried over Na₂SO₄, filtered and concentrated in vacuo to give the crude, which was purified by silica gel chromatography (MeOH/DCM=1/20) to give (1S,2S)-2-(3-((trimethylsilyl)ethynyl)phenyl)cyclopropane-1-carboxamide (180 mg, 53.4%) as a yellow solid. ESI-MS [M+H]+: 258.1.

### Synthesis of rac-(1S*, 2S*)-2-(7-chloro-8-fluoroimidazo[1,5-a]pyridin-1-yl)cyclopropanecarboxylic acid

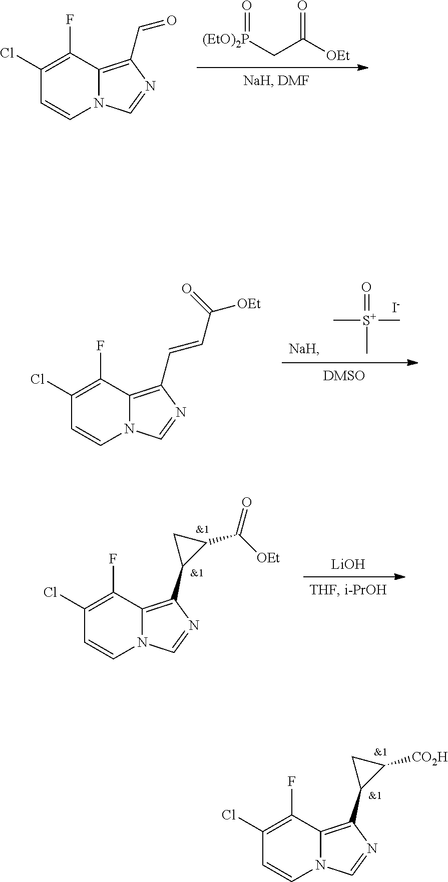

### Synthesis of ethyl 3-(7-chloro-8-fluoroimidazo[1,5-a]pyridin-1-yl)acrylate

To a solution of ethyl 2-(diethoxyphosphoryl)acetate (6.8 g, 30.3 mmol) in dry DMF (100 mL) cooled at 0° C. under N₂ was added NaH (60%, 910 mg, 37.88 mmol) in portions. The reaction mixture was stirred at 0° C. for 1 h before 7-chloro-8-fluoroimidazo[1,5-a]pyridine-1-carbaldehyde (5 g, 25.25 mmol) was added dropwise. The resulting mixture was allowed to warm to room temperature and stirred overnight. Once the reaction was complete by TLC, the reaction was quenched with saturated NH₄Cl solution and extracted with ethyl acetate (50 mL×3). The combined organic layers were washed with brine, dried over anhydrous sodium sulphate, filtered, and concentrated in vacuo. The residue was purified by silica gel column chromatography (petroleum ether:ethyl acetate=3:1) to afford ethyl 3-(7-chloro-8-fluoroimidazo[1,5-a]pyridin-1-yl)acrylate (3.2 g, 47%) as a yellow solid. ESI-MS [M+H]⁺ 269.1

### Synthesis of rac-(1S*, 2S*)-ethyl 2-(7-chloro-8-fluoroimidazo[1,5-a]pyridin-1-yl)cyclopropanecarboxylate

To a solution of trimethylsulfoxonium iodide (5.25 g, 23.88 mmol) in dry DMSO (100 mL) was added NaH (60%, 860 mg, 35.82 mmol) in portions at 0° C. The reaction mixture was stirred at this temperature for 30 min before ethyl 3-(7-chloro-8-fluoroimidazo[1,5-a]pyridin-1-yl)acrylate (3.2 g, 11.94 mmol) was added. The resulting mixture was stirred at room temperature overnight then quenched with saturated NH₄Cl solution, extracted with ethyl acetate (50 mL×3). The combined organic layers were washed with brine, dried over sodium sulfate, filtrated, concentrated in vacuo and purified by chromatography (petrol ether:ethyl acetate=3:1) to give rac-(1S*, 2S*)-ethyl 2-(7-chloro-8-fluoroimidazo[1,5-a]pyridin-1-yl)cyclopropanecarboxylate (440 mg, 13%) as a yellow solid. ESI-MS [M+H]⁺ 283.1

### Synthesis of rac-(1S*, 2S*)-2-(7-chloro-8-fluoroimdazo[1,5-a]pyridin-1-yl)cyclopropanecarboxylic acid

To a solution of rac-(1S*, 2S*)-ethyl 2-(7-chloro-8-fluoroimidazo[1,5-a]pyridin-1-yl)cyclopropanecarboxylate (440 mg, 1.56 mmol) in THF (10 mL), iPrOH (10 mL) and water (5 mL) was added lithium hydroxide hydrate (197 mg, 4.68 mmol). The resulting reaction mixture was stirred at room temperature for 4 hrs. After evaporation, the residue was diluted with water (5 mL) and acidified with 1 N HCl to pH˜5. The precipitate was filtered and dried under vacuum to give rac-(1S*,2S*)-2-(7-chloro-8-fluoroimidazo[1,5-a]pyridin-1-yl)cyclopropanecarboxylic acid (280 mg, 71%) as a white solid. ESI-MS 255.0 [M+H]⁺, ¹H NMR (400 MHz, DMSO) δ 8.37 (d, J=2.4 Hz, 1H), 8.16 (d, J=7.2 Hz, 1H), 6.72 (t, J=6.8 Hz, 1H), 2.66-2.64 (m, 1H), 1.97-1.93 (m, 1H), 1.51-1.43 (m, 2H).

### Synthesis of rac-(1S*,2S*)-2-(3-bromophenyl)cyclopropanecarboxylic acid

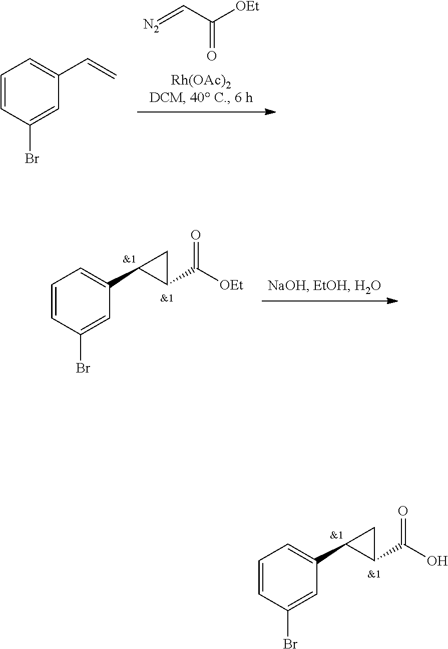

### Synthesis of rac-(1S*,2S*)-ethyl 2-(3-bromophenyl)cyclopropanecarboxylate

To a solution of 1-bromo-3-vinylbenzene (5 g, 27.32 mmol) in DCM (50 mL) was added Rh(OAc)₂ (765 mg, 2.73 mmol). The mixture was heated to 40° C. and a solution of ethyl diazoacetate (15.7 g, 136.66 mmol) in DCM (300 mL) was added dropwise over 6 hrs. After the addition, the reaction mixture was cooled to room temperature and filtered through a short pad of Celite. The filtrate was concentrated in vacuo and purified by silica gel column chromatography (petroleum ether:ethyl acetate=20:1) to afford rac-(1S*,2S*)-ethyl 2-(3-bromophenyl)cyclopropanecarboxylate (3.9 g, 53%) as a colorless oil. ESI-MS [M+H]⁺ 269.0

### Synthesis of rac-(1S*,2S*)-2-(3-bromophenyl)cyclopropanecarboxylic acid

To a solution of trans-ethyl 2-(3-bromophenyl)cyclopropanecarboxylate (1.8 g, 6.7 mmol) in EtOH (30 mL) was added sodium hydroxide (550 mg, 13.8 mmol). After stirring at room temperature for 1 hr, the mixture was cooled to 0° C., and HCl (1M, 10 mL) was added. The suspension was filtered and the filter cake was washed with water and dried under vacuum to afford rac-(1S*,2S*)-2-(3-bromophenyl)cyclopropanecarboxylic acid (1.4 g, 88%) as a white solid. ESI-MS [M+H]⁺ 242.8, ¹H NMR (400 MHz, CDCl₃): δ 7.35 (d, J=7.6 Hz, 1H), 7.25 (s, 1H), 7.15 (d, J=8.0 Hz, 1H), 7.04 (d, J=7.6 Hz, 1H), 2.59-2.54 (m, 1H), 1.92-1.82 (m, 1H), 1.69-1.64 (m, 1H), 1.42-1.36 (m, 1H).

### Synthesis of rac-(1S*,2S*)-2-(3-cyclopropylphenyl)cyclopropanecarboxylic acid

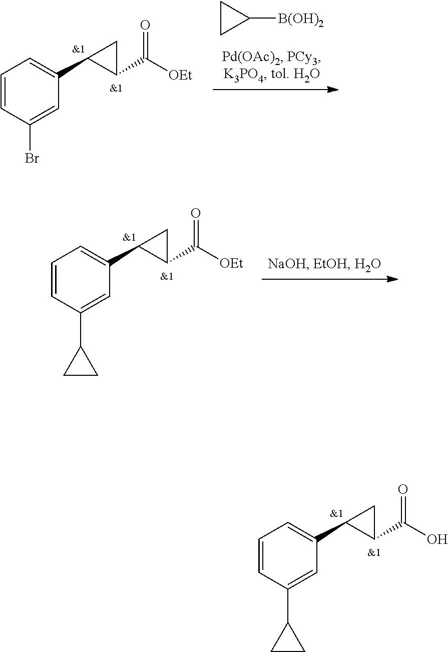

### Synthesis of rac-(1S*,2S*)-ethyl 2-(3-cyclopropylphenyl)cyclopropanecarboxylate

To a solution of trans-ethyl 2-(3-bromophenyl)cyclopropanecarboxylate (2.0 g, 7.5 mmol) in tol/H₂O (40 mL/8 mL), was added cyclopropylboronic acid (9.7 g, 113 mmol), Pd(OAc)₂ (840 mg, 3.75 mmol), tricyclohexyl phosphine (PCy₃, 2.1 g, 7.5 mmol and K₃PO₄ (4.8 g, 22.5 mmol). The mixture was stirred at 100° C. under N₂ overnight. Once the reaction was complete by TLC, the mixture was cooled to room temperature and quenched with water then extracted with ethyl acetate. The combined organic layers were washed with brine, dried and concentrated in vacuo. The residue was purified by silica gel column chromatography (petroleum ether:ethyl acetate=20:1) to afford rac-(1S*,2S*)-ethyl 2-(3-cyclopropylphenyl)cyclopropanecarboxylate (1.3 g, 87%) as a yellow oil.

### Synthesis of rac-(1S*,2S*)-2-(3-cyclopropylphenyl)cyclopropanecarboxylic acid

To a solution of trans-ethyl 2-(3-cyclopropylphenyl)cyclopropanecarboxylate (1.3 g, 5.65 mmol) in ethanol (10 mL) and water (4 mL) was added sodium hydroxide (452 mg, 11.3 mmol). After the mixture was stirred at room temperature for 1.5 hrs, the reaction was complete by TLC. The mixture was concentrated in vacuo. The residue was diluted with water and then acidified with 1M HCl to pH=5. The mixture was extracted with ethyl acetate and the combined organic layers were washed with brine then concentrated in vacuo to afford rac-(1S*,2S*)-2-(3-cyclopropylphenyl)cyclopropanecarboxylic acid (1.05 g, 92%) as a yellow solid. ESI-MS [M−H]-201.0, ¹H NMR (400 MHz, DMSO) δ 12.27 (s, 1H), 7.13 (t, J=7.6 Hz, 1H), 6.90-6.87 (m, 3H), 2.36-2.32 (m, 1H), 1.89-1.77 (m, 2H), 1.42-1.38 (m, 1H), 1.34-1.30 (m, 1H), 0.93-0.88 (m, 2H), 0.68-0.64 (m, 2H).

### Synthesis of rac-(1S*,2S*)-2-(3-cyclopropylphenyl)cyclopropanecarboxylic acid

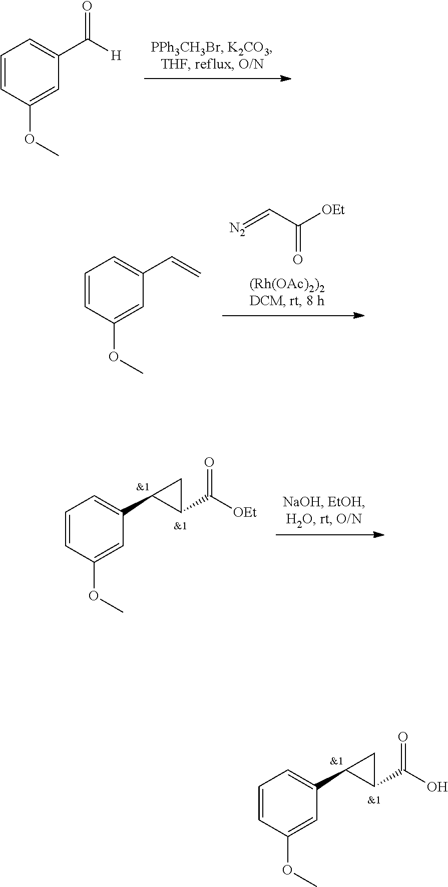

### Synthesis of 1-methoxy-3-vinylbenzene

3-methoxybenzaldehyde (20 g, 147 mmol) was added to a stirred suspension of potassium carbonate (36.5 g, 264.6 mmol) and methyltriphenylphonium bromide (78.7 g, 220.5 mmol) in anhydrous THF (300 mL) under nitrogen. The reaction was stirred at reflux overnight. After cooling to room temperature, the mixture was filtered, and the filtrate was concentrated in vacuo. The residue was purified by silica gel column chromatography (petroleum ether) to afford 1-methoxy-3-vinylbenzene (15.6 g, 79%) as a colourless oil. ESI-MS [M+H]⁺ 135.1

### Synthesis of rac-(1S*,2S*)-ethyl 2-(3-methoxyphenyl)cyclopropanecarboxylate

To a suspension of 1-methoxy-3-vinylbenzene (9.6 g, 71.6 mmol) and diacetoxyrhodium (2.5 g, 5.72 mmol) in DCM (100 mL) was added ethyl diazoacetate (20 g, 173.9 mmol) in DCM (150 mL) dropwise over 8 hours. After the addition, the reaction mixture was filtered, and the filtrate was concentrated in vacuo. The residue was purified by silica gel column chromatography (petroleum ether:ethyl acetate=20:1) to afford rac-(1S*,2S*)-ethyl 2-(3-methoxyphenyl)cyclopropanecarboxylate (5.8 g, 37%) as a colorless oil.

### Synthesis of rac-(1S*,2S*)-2-(3-methoxyphenyl)cyclopropanecarboxylic acid

To a solution of trans-ethyl 2-(3-methoxyphenyl)cyclopropanecarboxylate (5.8 g, 26.3 mmol) in ethanol (40 mL) and water (10 mL) was added sodium hydroxide (3.1 g, 71.9 mmol). The mixture was stirred at room temperature overnight. Once the reaction was complete by LCMS, the mixture was concentrated in vacuo. The residue was diluted with water (20 mL) and then acidified with 1M HCl to pH=1-2. The resulting precipitate was collected by filtration and dried under vacuum to afford rac-(1S*,2S*)-2-(3-methoxyphenyl)cyclopropanecarboxylic acid (4.26 g, 85%) as a white solid. ESI-MS [2M−H]⁻ 382.9, 1H NMR (400 MHz, CDCl₃) δ 7.20 (t, J=8.0 Hz, 1H), 6.75 (d, J=8.8 Hz, 1H), 6.69 (d, J=7.6 Hz, 1H), 6.65 (s, 1H), 3.80 (s, 3H), 2.60-2.55 (m, 1H), 1.94-1.86 (m, 1H), 1.68-1.62 (m, 1H), 1.45-1.35 (m, 1H).

### Synthesis of (6-cyclopropylimidazo[1,2-a]pyridin-2-yl)methanamine

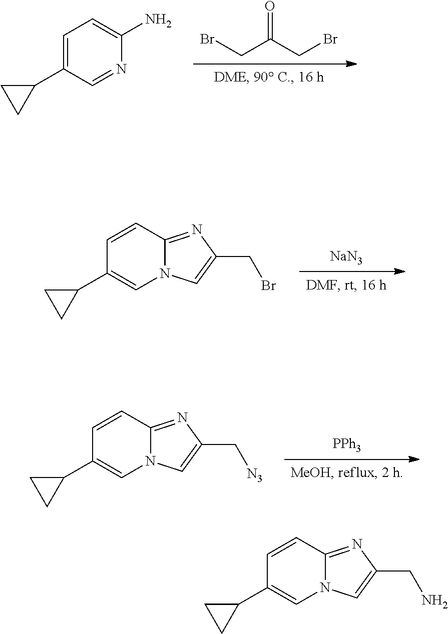

### Synthesis of 2-(bromomethyl)-6-cyclopropylimidazo[1,2-a]pyridine

A mixture of 5-cyclopropylpyridin-2-amine (670 mg, 5.0 mmol) and 1,3-dibromopropan-2-one (1.61 g, 7.5 mmol) in DME (20.0 mL) was stirred at 90° C. under N₂ for 16 h. The reaction mixture was cooled to room temperature, quenched with sat·NaHCO₃ solution (50 mL) and extracted with EtOAc (3×50 ml). The combined organic layers were washed with brine (50 mL), dried over Na₂SO₄ and concentrated in vacuo. The crude residue was purified by column chromatography (eluent: DCM/MeOH=50/1˜30/1) to give the product 2-(bromomethyl)-6-cyclopropylimidazo[1,2-a]pyridine (880 mg, 70%) as a yellow solid. ESI-MS [M+H]⁺: 252.2.

### Synthesis of 2-(azidomethyl)-6-cyclopropylimidazo[1,2-a]pyridine

A mixture of 2-(bromomethyl)-6-cyclopropylimidazo[1,2-a]pyridine (753 mg, 3 mmol) and NaN₃ (244 mg, 3.75 mmol) in DMF (10.0 mL) was stirred at room temperature under N₂ for 16 h. The mixture was diluted with EtOAc (100 mL) and washed with brine (3×50 mL). The organic layers were dried over Na₂SO₄ and concentrated in vacuo to give 2-(azidomethyl)-6-cyclopropylimidazo[1,2-a]pyridine (730 mg, crude) as a yellow solid, which was used for the next step without purification. ESI-MS [M+H]⁺: 214.2.

### Synthesis of (6-cyclopropylimidazo[1,2-a]pyridin-2-yl)methanamine

A mixture of 2-(azidomethyl)-6-cyclopropylimidazo[1,2-a]pyridine (730 mg, crude) and PPh₃ (983 mg, 3.75 mmol) in MeOH (25 mL) was stirred at reflux for 2 h. The mixture was concentrated in vacuo and purified by preparative TLC (eluent: DCM/MeOH=10/1) to give (6-cyclopropylimidazo[1,2-a]pyridin-2-yl)methanamine (450 mg, 81% over 2 steps) as a yellow oil. ESI-MS [M+H]+: 188.2

### Synthesis of 2-((tert-butyldimethylsilyl)oxy)-N-((6-cyclopropylimidazo[1,2-a]pyridin-2-yl)methyl)ethan-1-amine

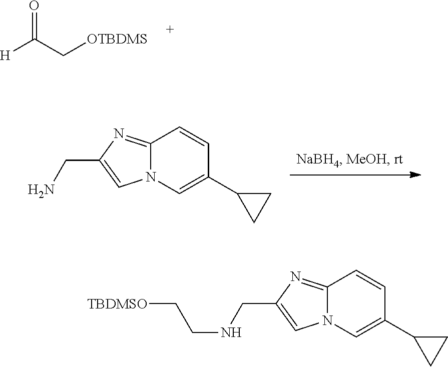 [SMILES: CC(C)(C)[Si](C)(C)OCC=O ; NCc1cn2cc(C3CC3)ccc2n1 ; "NaBH4, MeOH, rt" ; CC(C)(C)[Si](C)(C)OCCNCc1cn2cc(C3CC3)ccc2n1]

(6-cyclopropylimidazo[1,2-a]pyridin-2-yl)methanamine (50 mg, 0.28 mmol) and 2-((tert-butyldimethylsilyl)oxy)acetaldehyde (76 μL, 0.40 mmol) were dissolved in MeOH (1.0 mL) and stirred at room temperature for 2 h. NaBH₄ (30 mg, 0.80 mmol) was then added and the reaction stirred at room temperature for a further 1 h. The reaction was then quenched with water (5 mL) and extracted into DCM (3×10 mL), the combined organics were dried over MgSO₄ then concentrated in vacuo. The residue was purified by silica gel chromatography, eluting with a gradient of 0-20% (7N NH₃ in MeOH) in EtOAc to give the title compound (47 mg, 51%). This was used directly without further purification.

### Synthesis of 1-(6-cyclopropylimidazo[1,2-a]pyridin-2-yl)-2,2,2-trifluoroethan-1-one

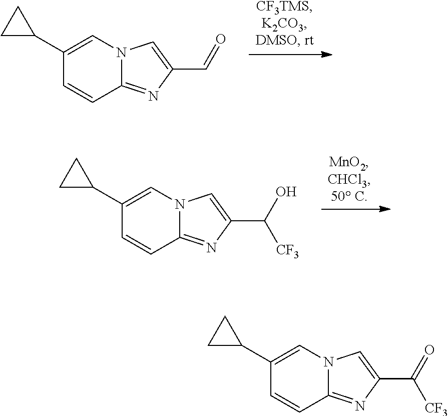 [SMILES: O=Cc1cn2cc(C3CC3)ccc2n1 ; "CF3TMS,K2CO3,DMSO, rt" ; OC(c1cn2cc(C3CC3)ccc2n1)C(F)(F)F ; "MnO2,CHCl3,50° C." ; O=C(c1cn2cc(C3CC3)ccc2n1)C(F)(F)F]

### Synthesis of 1-(6-cyclopropylimidazo[1,2-a]pyridin-2-yl)-2,2,2-trifluoroethan-1-ol

To a solution of 6-cyclopropylimidazo[1,2-a]pyridine-2-carbaldehyde (100 mg, 0.54 mmol) and trimethyl(trifluoromethyl)silane (99 mg, 0.70 mmol) in DMSO (2.0 mL) was added K₂CO₃ (7.4 mg, 0.05 mmol) and the reaction mixture was stirred at room temperature for 16 h. The mixture was poured onto ice water and extracted with ethyl acetate (3×20 mL). The organics were dried over MgSO₄ and concentrated in vacuo to give the title compound (100 mg, 73%) which was used without further purification. ESI-MS (M+H)+: 257.2, ¹H NMR (400 MHz, CDCl₃) δ 7.89 (s, 1H), 7.55 (s, 1H), 7.50 (d, J=9.5 Hz, 1H), 7.00 (dd, J=1.7, 9.3 Hz, 1H), 5.23-5.17 (m, 1H), 1.94-1.86 (m, 1H), 1.02-0.96 (m, 2H), 0.71-0.66 (m, 2H). OH not observed

### Synthesis of 1-(6-cyclopropylimidazo[1,2-a]pyridin-2-yl)-2,2,2-trifluoroethan-1-one

A suspension of 1-(6-cyclopropylimidazo[1,2-a]pyridin-2-yl)-2,2,2-trifluoroethan-1-ol (400 mg, 1.6 mmol), manganese(IV) oxide (1.4 g, 16 mmol) in chloroform (15 mL) was stirred at 50° C. for 16 h. The mixture was cooled to room temperature, filtered through Celite®, washed with DCM (30 mL) and concentrated in vacuo to give the title compound (350 mg, 88%) which was used without further purification. ESI-MS (M+H)+: 273.2

### Synthesis of (6-isopropylimidazo[1,2-a]pyridin-2-yl)methanamine

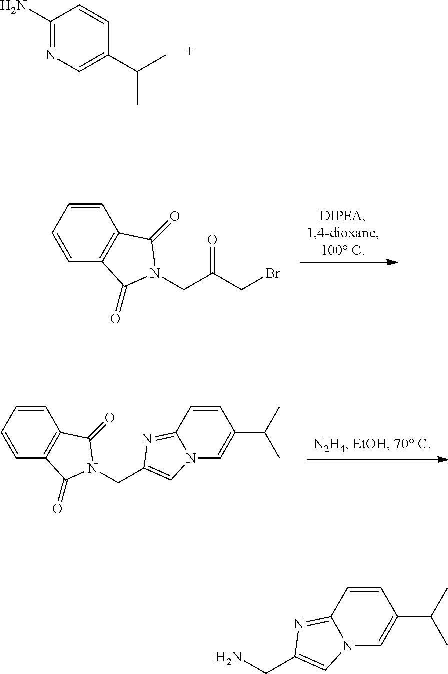

### Synthesis of 2-((6-Isopropylimidazo[1,2-a]pyridin-2-yl)methyl)isoindoline-1,3-dione

A solution of 5-isopropylpyridin-2-amine (500 mg, 3.7 mmol), 2-(3-bromo-2-oxopropyl)isoindoline-1,3-dione (1100 mg, 4.0 mmol) and DIPEA (0.96 mL, 5.5 mmol) in 1,4-dioxane (37 mL) was stirred at 100° C. for 16 h. The mixture was cooled to room temperature and concentrated in vacuo. The residue was purified by silica gel chromatography eluting with a gradient of 50-100% EtOAc in cyclohexane to give the title compound (480 mg, 41%) as a red oil. ESI-MS (M+H)+: 320.1, ¹H NMR (400 MHz, DMSO) 8.38 (1H, s), 8.03-7.94 (4H, m), 7.88 (1H, s), 7.48 (1H, d, J=9.3 Hz), 7.27 (1H, dd, J=1.8, 9.3 Hz), 4.97 (2H, s), 3.01-2.93 (1H, m), 1.30 (6H, d, J=6.8 Hz);

### Synthesis of (6-Isopropylimidazo[1,2-a]pyridin-2-yl)methanamine

Using a similar procedure to that for 3-(2-(aminomethyl)-6-cyclopropylimidazo[1,2-a]pyridin-8-yl)oxetan-3-ol, (6-isopropylimidazo[1,2-a]pyridin-2-yl)methanamine (272 mg, 97%) was synthesised from 2-((6-isopropylimidazo[1,2-a]pyridin-2-yl)methyl)isoindoline-1,3-dione (480 mg, 1.50 mmol) using hydrazine hydrate. ESI-MS (M+H)+: 190.1, ¹H NMR (400 MHz, DMSO) δ 8.33-8.33 (m, 1H), 7.69 (s, 1H), 7.41-7.38 (m, 1H), 7.18-7.15 (m, 1H), 3.80-3.79 (m, 2H), 2.95-2.86 (m, 1H), 1.26-1.23 (m, 6H).

### Synthesis of 2-(azidomethyl)-N,N-dimethylimidazo[1,2-a]pyridin-6-amine

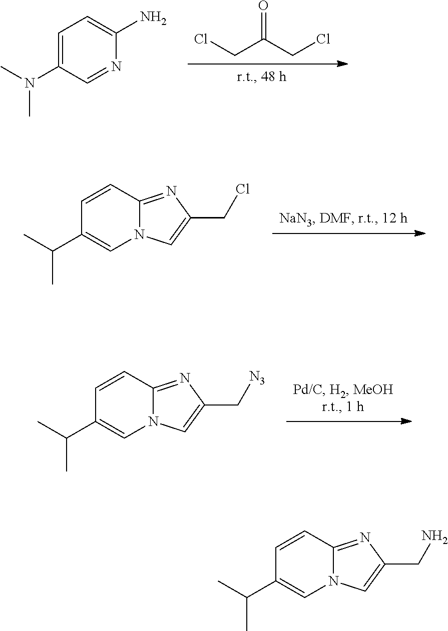

### Synthesis of 2-(2-((tert-butyldimethylsilyl)oxy)ethoxy)-4,6-dichloropyrimidine

A solution of N⁵,N⁵-dimethylpyridine-2,5-diamine (1 g, 7.3 mmol) in 1,3-dichloropropan-2-one (2.9 g, 21.9 mmol) was stirred at room temperature for 48h. The reaction mixture was used into next step without purification. ESI-MS [M+H]+: 210.1.

### Synthesis of 2-(azidomethyl)-N,N-dimethylimidazo[1,2-a]pyridin-6-amine

To the solution from previous step was added DMF (30 mL), followed by NaN₃ (4.7 g, 73 mmol). The reaction was stirred at room temperature for 12 h. The reaction was quenched with water (50 mL) and extracted with EtOAc (3×50 mL). The combined organic layers were washed with brine (50 mL), dried over anhydrous Na₂SO₄ and concentrated in vacuo to give the crude product, which was purified by silica gel chromatography (eluent: PE/EtOAc=1/1) to give 2-(azidomethyl)-N,N-dimethylimidazo[1,2-a]pyridin-6-amine (300 mg, 19% (2 steps)) as a yellow solid. ESI-MS [M+H]+: 217.2.

### Synthesis of 2-(aminomethyl)-N,N-dimethylimidazo[1,2-a]pyridin-6-amine

A solution of 2-(azidomethyl)-N,N-dimethylimidazo[1,2-a]pyridin-6-amine (300 mg, 1.39 mmol) and Pd/C (30 mg) in MeOH (10 mL) was stirred at room temperature under H₂ atmosphere for 1 h. The reaction mixture was filtered through a pad of celite, washed with MeOH (3×30 mL). The filtrate was concentrated in vacuo to give 2-(aminomethyl)-N,N-dimethylimidazo[1,2-a]pyridin-6-amine (210 mg, 79.5%) as a yellow solid, which was used in the next step without further purification. ESI-MS [M+H]+: 191.2.

### Synthesis of (3-chloro-6-cyclopropylimidazo[1,2-a]pyridin-2-yl)methanamine

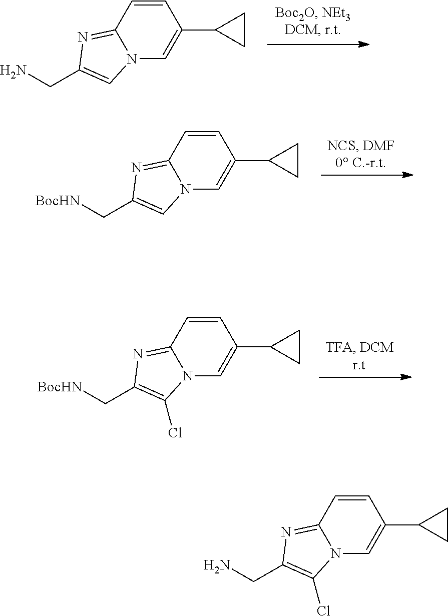

### Synthesis of tert-butyl ((6-cyclopropylimidazo[1,2-a]pyridin-2-yl)methyl)carbamate

A mixture of (6-cyclopropylimidazo[1,2-a]pyridin-2-yl)methanamine (1.0 g, 5.3 mmol), di-tert-butyl decarbonate (1.4 g, 6.4 mmol) and NEt₃ (1.1 mL, 8.0 mmol) in DCM (55 mL) was stirred at room temperature for 18 h. A solution of NaHCO₃ (sat·aq·, 50 mL) was added and the mixture was extracted with DCM (3×50 mL). The combined organics were dried (MgSO₄), filtered and concentrated in vacuo. The residue was purified by column chromatography on silica gel eluting with 20-100% EtOAc in cyclohexane to give the title compound (930 mg, 60%) as a yellow solid. ESI-MS (M+H)+: 288.2, ¹H NMR (400 MHz, DMSO) δ 8.38 (s, 1H), 7.63 (s, 1H), 7.40 (d, J=9.3 Hz, 1H), 7.32 (dd, J=5.7, 5.7 Hz, 1H), 7.00 (dd, J=1.9, 9.2 Hz, 1H), 4.25 (d, J=6.1 Hz, 2H), 2.01-1.93 (m, 1H), 1.45 (s, 9H), 1.00-0.94 (m, 2H), 0.75-0.70 (m, 2H).

### Synthesis of tert-butyl ((3-chloro-6-cyclopropylimidazo[1,2-a]pyridin-2-yl)methyl)carbamate

NCS (0.10 g, 0.76 mmol) was added to a solution of tert-butyl ((6-cyclopropylimidazo[1,2-a]pyridin-2-yl)methyl)carbamate (0.20 g, 0.70 mmol) in DMF (5.0 mL) at 0° C. under a N₂ atmosphere. The mixture was stirred at 0° C. for 4 h then quenched with water. The mixture was extracted with EtOAc. The combined organic layers were dried (MgSO₄), filtered and concentrated in vacuo to give the title compound (0.11 g, 49%). ESI-MS (M+H)+: 322.3, ¹H NMR (400 MHz, DMSO) δ 8.14 (s, 1H), 7.55 (dd, J=0.6, 9.4 Hz, 1H), 7.32-7.26 (m, 1H), 7.11 (dd, J=1.7, 9.1 Hz, 1H), 4.30 (d, J=5.7 Hz, 2H), 2.18-2.10 (m, 1H), 1.44 (s, 9H), 1.05-0.99 (m, 2H), 0.84-0.78 (m, 2H).

### Synthesis of (3-chloro-6-cyclopropylimidazo[1,2-a]pyridin-2-yl)methanamine

A solution of tert-butyl ((3-chloro-6-cyclopropylimidazo[1,2-a]pyridin-2-yl)methyl)carbamate (0.11 g, 0.34 mmol) in TFA (0.26 mL) and DCM (5.0 mL) was stirred at room temperature for 3 h. The mixture was loaded onto an SCX cartridge, washed with MeOH and eluted with 7 N NH₃ in MeOH to give the title compound (0.060 g, 79%) which was used directly in the next step. ESI-MS (M+H)+: 222.2

### Synthesis of 2-(aminomethyl)-6-cyclopropylimidazo[1,2-a]pyridine-8-carbonitrile

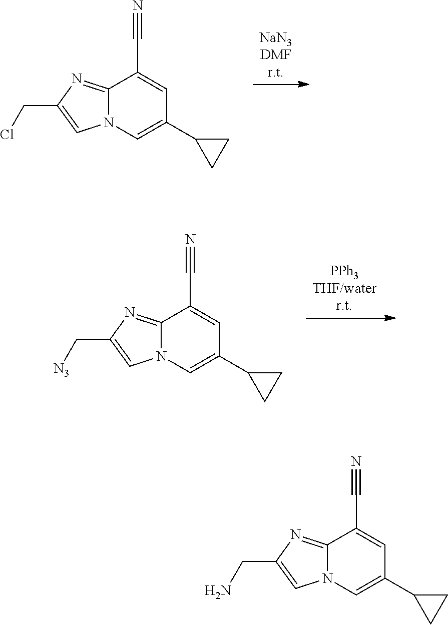 [SMILES: N#Cc1cc(C2CC2)cn2cc(CCl)nc12 ; "NaN3DMFr.t." ; N#Cc1cc(C2CC2)cn2cc(CN=[N+]=[N-])nc12 ; "PPh3THF/waterr.t." ; N#Cc1cc(C2CC2)cn2cc(CN)nc12]

### Synthesis of 2-(azidomethyl)-6-cyclopropylimidazo[1,2-a]pyridine-8-carbonitrile

2-(Chloromethyl)-6-cyclopropylimidazo[1,2-a]pyridine-8-carbonitrile (630 mg, 2.70 mmol) and NaN₃ (230 mg, 3.5 mmol) were combined in DMF (5.0 mL) and stirred at room temperature under a nitrogen atmosphere for 18 h. The mixture was diluted with EtOAc (50 mL) and washed with water (2×50 mL) and brine (50 mL), then dried over MgSO₄, filtered and concentrated in vacuo to give the title compound (580 mg, 89%) as a purple oil. ESI-MS [M+H]+: 239.2, ¹H NMR (400 MHz, DMSO) δ 8.72 (dd, J=0.5, 1.7 Hz, 1H), 8.02 (s, 1H), 7.80 (d, J=1.8 Hz, 1H), 4.56 (s, 2H), 2.03-1.96 (m, 1H), 0.99-0.94 (m, 2H), 0.80-0.75 (m, 2H).

### Synthesis of 2-(aminomethyl)-6-cyclopropylimidazo[1,2-a]pyridine-8-carbonitrile

2-(Azidomethyl)-6-cyclopropylimidazo[1,2-a]pyridine-8-carbonitrile (580 mg, 2.4 mmol) and triphenyl phosphine (1.3 g, 4.8 mmol) were combined in THF (11 mL) and water (1.0 mL) and stirred at room temperature for 24 h. The mixture was then concentrated in vacuo, dissolved in DCM and treated with HCl in dioxane (4.0 M). The precipitate was collected by filtration and washed with DCM to give the title compound (630 mg, 90%) as a pale yellow solid. ESI-MS [M+H]+: 213.2, ¹H NMR (400 MHz, DMSO) δ 8.81 (s, 1H), 8.38 (br s, 3H), 8.06 (s, 1H), 7.85 (s, 1H), 4.19 (d, J=5.6 Hz, 2H), 2.04-1.97 (m, 1H), 1.02-0.95 (m, 2H), 0.82-0.76 (m, 2H).

### Synthesis of 6-cyclopropyl-2-(hydroxymethyl)imidazo[1,2-a]pyridine-8-carbonitrile

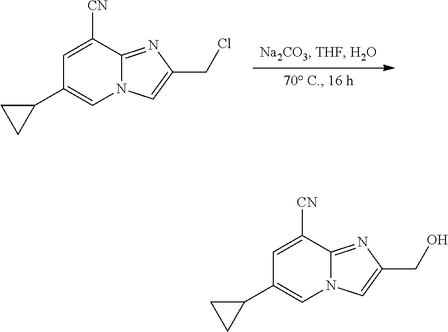

A mixture of 2-(chloromethyl)-6-cyclopropylimidazo[1,2-a]pyridine-8-carbonitrile (0.55 g, 2.0 mmol) and Na₂CO₃ (1.3 g, 12 mmol) in a mixed solvent of THF/H₂O (V/V=1/1, 20 mL) was stirred at 70° C. for 16 h. After cooling to room temperature, the reaction mixture was extracted with EtOAc/MeOH (10/1, 3×30 mL). The combined organic layers were concentrated and purified by prep-TLC (eluent: DCM/MeOH=20/1) to give 6-cyclopropyl-2-(hydroxymethyl)-imidazo[1,2-a]pyridine-8-carbonitrile (0.17 g, 40%) as a yellow solid. ESI-MS [M+H]⁺: 214.2

### Synthesis of (2-(aminomethyl)-6-cyclopropylimidazo[1,2-a]pyridin-8-yl)methanol hydrochloride

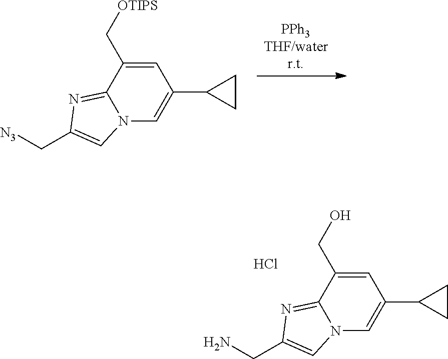

A mixture of 2-(azidomethyl)-6-cyclopropyl-8-(((triisopropylsilyl)oxy)methyl)imidazo[1,2-a]pyridine (0.80 g, 2.0 mmol), PPh₃ (1.2 g, 4.5 mmol) and water (1.0 mL) in THF (10 mL) was stirred at room temperature for 18 h. The mixture was concentrated in vacuo. The material was divided, and 0.95 g of the 2.0 g residue was dissolved in DCM and washed with HCl solution (2.0 M aq·) and water. The combined aqueous layers were concentrated in vacuo. The residue was dissolved in MeOH and MP-carbonate resin (0.4 g) was added. The mixture was stirred gently for 1 h then filtered. The filtrate was concentrated in vacuo to give the title compound (0.15 g, 20%) as a yellow gum. ESI-MS (M+H)+: 218.2, ¹H NMR (400 MHz, DMSO) δ 8.98-8.85 (m, 3H), 8.80 (s, 1H), 8.34 (s, 1H), 7.66 (s, 1H), 4.89 (s, 2H), 4.43-4.35 (m, 2H), 3.22 (s, 1H), 2.19-2.10 (m, 1H), 1.15-1.07 (m, 2H), 0.88-0.84 (m, 2H).

### Synthesis of 1-(2-(aminomethyl)-6-cyclopropylimidazo[1,2-a]pyridin-8-yl)ethan-1-one

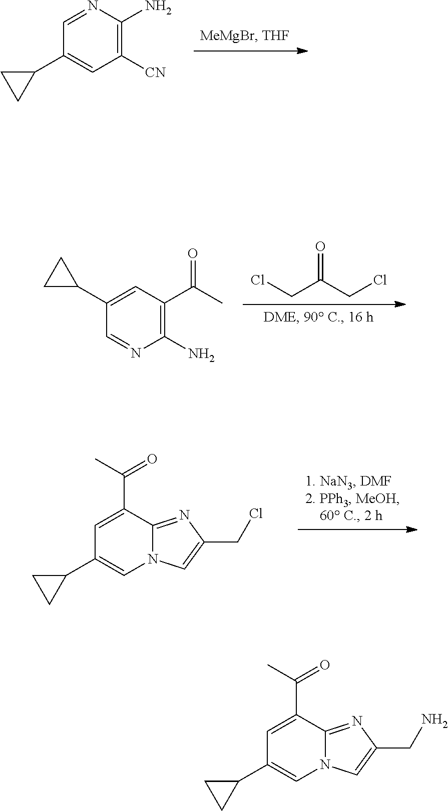

### Synthesis of 1-(2-amino-5-cyclopropylpyridin-3-yl)ethan-1-one

To a solution of 2-amino-5-cyclopropylnicotinonitrile (400 mg, 2.52 mmol) in THF (15 mL) was added CH₃MgBr (6.72 ml, 3M in THF, 20.16 mmol) drop-wise at 0° C. Then the mixture was stirred at 60° C. for 2.5 h, quenched with sat·NH₄Cl solution (30 mL) and extracted with EtOAc/MeOH (10/1) (3×30 mL). The organic layers were concentrated in vacuo to give the crude product, which was purified by silica gel chromatography (eluent: DCM/MeOH=0˜5%) to give 1-(2-amino-5-cyclopropylpyridin-3-yl)ethan-1-one as a light yellow solid (300 mg, 67%). ESI-MS [M+H]⁺: 177.2.

### Synthesis of 1-(2-(chloromethyl)-6-cyclopropylimidazo[1,2-a]pyridin-8-yl)ethan-1-one

A solution of 1-(2-amino-5-cyclopropylpyridin-3-yl)ethan-1-one (600 mg, 3.41 mmol) and 1,3-dichloropropan-2-one (859 mg, 6.82 mmol) in DME (20 mL) was stirred at 90° C. for 16 h under N₂. The reaction was cooled to room temperature and concentrated in vacuo to give the crude product, which was purified by silica gel chromatography (eluent: DCM/MeOH=0˜20%) to give 1-(2-(chloromethyl)-6-cyclopropylimidazo[1,2-a]pyridin-8-yl)ethan-1-one as a brown solid (550 mg, 65%). ESI-MS [M+H]⁺: 249.1.

### Synthesis of 1-(2-(azidomethyl)-6-cyclopropylimidazo[1,2-a]pyridin-8-yl)ethan-1-one

A solution of 1-(2-(chloromethyl)-6-cyclopropylimidazo[1,2-a]pyridin-8-yl)ethan-1-one (550 mg, 2.02 mmol) and NaN₃ (180 mg, 2.77 mmol) in DMF (10 mL) was stirred at room temperature for 3 h under N₂. The mixture was diluted with EtOAc (50 mL) and washed with H₂O (3×50 mL). The organic layers were concentrated in vacuo to give 1-(2-(azidomethyl)-6-cyclopropylimidazo[1,2-a]pyridin-8-yl)ethan-1-one as a brown solid (450 mg, crude), which was used into next step directly. ESI-MS [M+H]⁺: 256.2.

### Synthesis of 1-(2-(aminomethyl)-6-cyclopropylimidazo[1,2-a]pyridin-8-yl)ethan-1-one

A solution of 1-(2-(azidomethyl)-6-cyclopropylimidazo[1,2-a]pyridin-8-yl)ethan-1-one (450 mg, crude) and PPh₃ (692 mg, 2.64 mmol) in MeOH (20 mL) was stirred at 60° C. for 2 h. The mixture was cooled to room temperature and concentrated in vacuo. The residue was purified by column chromatography (eluent: DCM/MeOH=0˜20%) to give 1-(2-(aminomethyl)-6-cyclopropylimidazo[1,2-a]pyridin-8-yl)ethan-1-one as a light yellow oil (320 mg, 79%). ESI-MS [M+H]⁺: 230.1

### Synthesis of 1-(2-(aminomethyl)-6-cyclopropylimidazo[1,2-a]pyridin-8-yl)ethan-1-ol

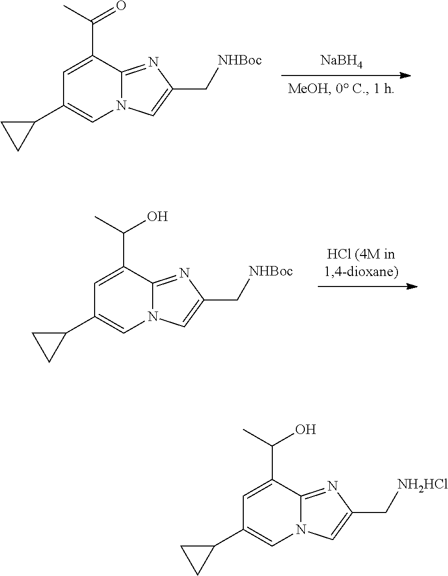

### Synthesis of tert-butyl ((6-cyclopropyl-8-(1-hydroxyethyl)imidazo[1,2-a]pyridin-2-yl)methyl)carbamate

To solution of tert-butyl ((8-acetyl-6-cyclopropylimidazo[1,2-a]pyridin-2-yl)methyl)carbamate (300 mg, 0.91 mmol) in MeOH (20 mL) was added NaBH₄ (17.1 mg, 0.45 mmol) at 0° C. After stirring at 0° C. for 1 h, the mixture was quenched with water (20 mL) and concentrated to give the crude product which was purified by preparative TLC (eluent: DCM/MeOH=15/1) to give tert-butyl ((6-cyclopropyl-8-(1-hydroxyethyl)imidazo[1,2-a]pyridin-2-yl)methyl)carbamate as a colourless oil (180 mg, 60%). ESI-MS [M+H]⁺: 332.1

### Synthesis of 1-(2-(aminomethyl)-6-cyclopropylimidazo[1,2-a]pyridin-8-yl)ethan-1-ol

A mixture of tert-butyl ((6-cyclopropyl-8-(1-hydroxyethyl)imidazo[1,2-a]pyridin-2-yl)methyl)carbamate (180 mg, 0.54 mmol) in HCl (4 M solution in 1,4-dioxane, 6 mL) was stirred at room temperature for 1 h. The mixture was concentrated in vacuo to give 1-(2-(aminomethyl)-6-cyclopropylimidazo[1,2-a]pyridin-8-yl)ethan-1-ol as the hydrochloric acid salt (180 mg, crude) as a yellow solid. ESI-MS [M+H]⁺: 232.1

### Synthesis of ethyl 3-(2-(aminomethyl)-6-cyclopropylimidazo[1,2-a]pyridin-8-yl)propanoate

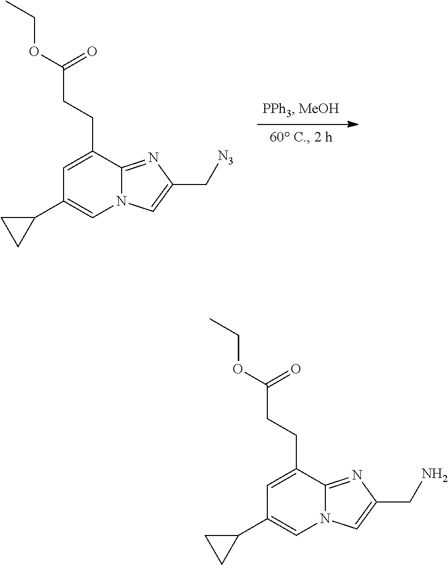

To a solution of ethyl 3-(2-(azidomethyl)-6-cyclopropylimidazo[1,2-a]pyridin-8-yl)propanoate (240 mg, 0.77 mmol) in MeOH (7 mL) was added PPh₃ (301 mg, 1.15 mmol). The resulting mixture was heated to 60° C. and stirred for 2 h under N₂. The reaction mixture was cooled to room temperature and concentrated in vacuo to give the crude product, which was purified by preparative TLC (eluent: MeOH/DCM=1/9) to give ethyl 3-(2-(aminomethyl)-6-cyclopropylimidazo[1,2-a]pyridin-8-yl)propanoate (130 mg, 59%) as yellow oil. ESI-MS [M+H]+: 288.2.

### Synthesis of (6-cyclopropyl-8-((2,2-diethoxyethoxy)methyl)imidazo[1,2-a]pyridin-2-yl)methanamine

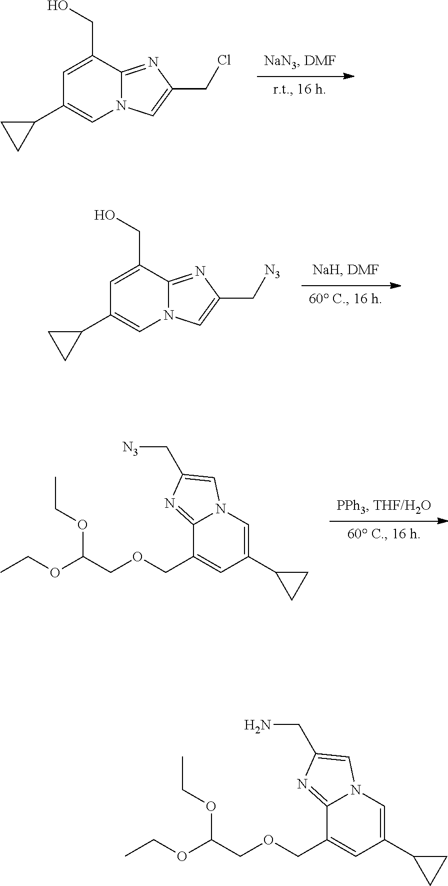

### Synthesis of 2-(azidomethyl)-6-cyclopropyl-8-((2,2-diethoxyethoxy)methyl)imidazo[1,2-a]pyridine

To a mixture of (2-(azidomethyl)-6-cyclopropylimidazo[1,2-a]pyridin-8-yl)methanol (1 g, 4.1 mmol) in DME (20 mL) was added NaH (0.39 g, 16.4 mmol) at 0° C. The reaction mixture was stirred at 0° C. for 30 min, 2-bromo-1,1-diethoxyethane (0.8 g, 4.1 mmol) was add slowly. The resulting mixture was stirred at 60° C. for 16 h under N₂. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give the crude, which was purified by silica gel chromatography (eluent: DCM/MeOH=100/1) to give 2-(azidomethyl)-6-cyclopropyl-8-((2,2-diethoxyethoxy)methyl)imidazo[1,2-a]pyridine (780 mg, 53%) as a white solid. ESI-MS [M+H]+: 360.2.

### Synthesis of (6-cyclopropyl-8-((2,2-diethoxyethoxy)methyl)imidazo[1,2-a]pyridin-2-yl)methanamine

A mixture of 2-(azidomethyl)-6-cyclopropyl-8-((2,2-diethoxyethoxy)methyl)imidazo[1,2-a]pyridine (780 mg, 2.17 mmol) and PPh₃ (1.1 g, 4.34 mmol) in a solution of THF (20 mL) and water (5 mL) was stirred at 60° C. for 16 h under N₂. Water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent: DCM/MeOH=100/1) to give (6-cyclopropyl-8-((2,2-diethoxyethoxy)methyl)imidazo[1,2-a]pyridin-2-yl)methanamine (450 mg, 62%) as a white solid. ESI-MS [M+H]+: 334.2.

### Synthesis of ethyl 2-(2-(aminomethyl)-6-cyclopropylimidazo[1,2-a]pyridin-8-yl)acetate

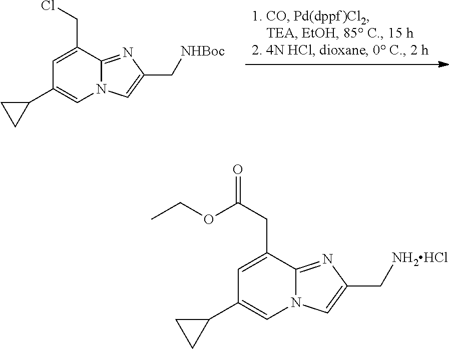

### Synthesis of ethyl 2-(2-(((tert-butoxycarbonyl)amino)methyl)-6-cyclopropylimidazo[1,2-a]pyridin-8-yl)acetate

A mixture of tert-butyl ((8-(chloromethyl)-6-cyclopropylimidazo[1,2-a]pyridin-2-yl)methyl)carbamate (0.35 g, 1.0 mmol), Pd(dppf)Cl₂ (82 mg, 0.11 mmol) and Et₃N (2.0 mL) in EtOH (10 mL) was stirred at 85° C. under CO for 15 h. After cooling to room temperature, the reaction mixture was concentrated in vacuo and purified by silica gel chromatography (eluent: DCM/MeOH=0˜10%) to give ethyl 2-(2-(((tert-butoxycarbonyl)amino)methyl)-6-cyclopropylimidazo[1,2-a]pyridin-8-yl)acetate (0.20 g, 54%) as a brown oil. ESI-MS [M+H]+: 374.2.

### Synthesis of ethyl 2-(2-(aminomethyl)-6-cyclopropylimidazo[1,2-a]pyridin-8-yl)acetate

A mixture of ethyl 2-(2-(((tert-butoxycarbonyl)amino)methyl)-6-cyclopropylimidazo[1,2-a]pyridin-8-yl)acetate (0.20 g, 0.54 mmol) in HCl (5 mL, 4 N in dioxane) was stirred at room temperature for 2 h. The reaction mixture was concentrated in vacuo to give ethyl 2-(2-(aminomethyl)-6-cyclopropylimidazo[1,2-a]pyridin-8-yl)acetate (0.15 g, quant) as a yellow solid (hydrochloric acid salt), which was used for the next step directly without further purification. ESI-MS [M+H]+: 274.2.

### Synthesis of 3-(2-(aminomethyl)-6-cyclopropylimidazo[1,2-a]pyridin-8-yl)oxetan-3-ol

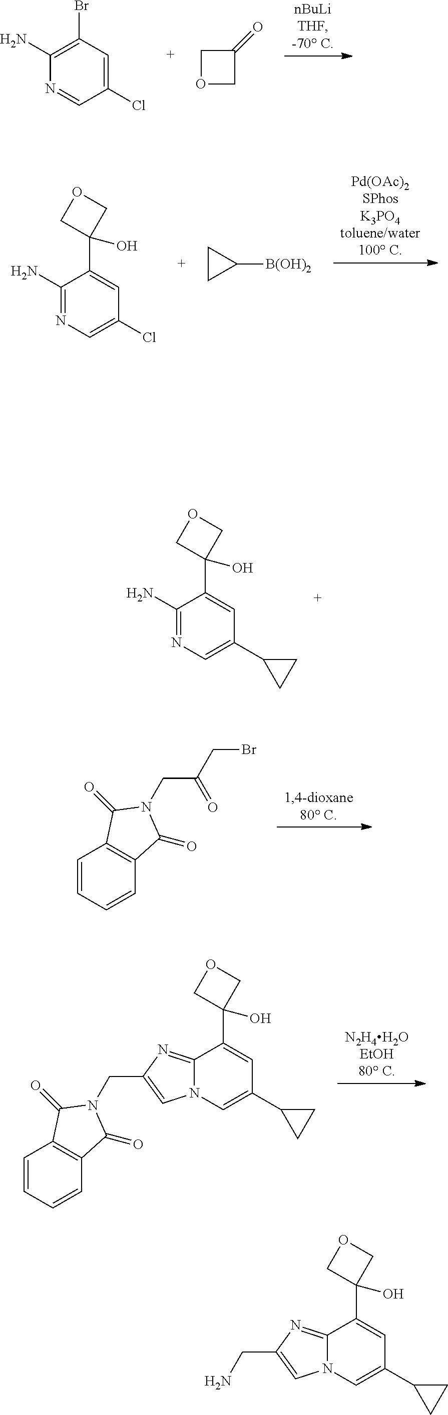

### Synthesis of 3-(2-amino-5-chloropyridin-3-yl)oxetan-3-ol

nBuLi in hexanes (9.2 mL, 22 mmol) was added dropwise to a stirred solution of 3-bromo-5-chloropyridin-2-amine (1.1 g, 5.5 mmol) in anhydrous THF (40 mL) at −70° C. under a nitrogen atmosphere. The mixture was stirred −70° C. for 1 h then a solution of oxetan-3-one (1.8 mL, 22 mmol) in THF (10 mL) was added slowly over 10 min. The resultant mixture was warmed to room temperature and stirred for 1 h. NH₄Cl (sat·aq·, 15 mL) was added and the mixture was extracted with EtOAc (3×25 mL). The combined organics were dried over MgSO₄, filtered and concentrated in vacuo. The residue was purified by column chromatography on silica gel, eluting with 0-10% MeOH in DCM to give the title compound (460 mg, 42%) as a pale brown solid. ESI-MS [M+H]+: 201.1, ¹H NMR (400 MHz, CDCl₃) δ 8.01 (d, J=2.4 Hz, 1H), 7.45 (d, J=2.4 Hz, 1H), 5.04-5.00 (m, 4H), 4.90-4.87 (m, 2H), 2.76 (br s, 1H).

### Synthesis of 3-(2-amino-5-cyclopropylpyridin-3-yl)oxetan-3-ol

A mixture of 3-(2-amino-5-chloropyridin-3-yl)oxetan-3-ol (550 mg, 2.8 mmol), cyclopropylboronic acid (350 mg, 4.1 mmol), SPhos (110 mg, 0.28 mmol), Pd(OAc)₂ (62 mg, 0.28 mmol) and K₃PO₄ (2.1 g, 9.6 mmol) in toluene (30 mL) and water (3.0 mL) was degassed with nitrogen then stirred at 100° C. for 18 h under a nitrogen atmosphere. The mixture was cooled and filtered through Celite® with EtOAc (75 mL). The filtrate was dried over MgSO₄, filtered and concentrated in vacuo. The residue was purified by column chromatography on silica gel, eluting with 0-10% MeOH in DCM to give the title compound (400 mg, 70%) as a pale yellow solid. ESI-MS [M+H]+: 207.1, ¹H NMR (400 MHz, CDCl₃) δ 7.85 (d, J=1.9 Hz, 1H), 7.16 (d, J=2.3 Hz, 1H), 5.07 (dd, J=0.8, 7.3 Hz, 2H), 4.89 (dd, J=0.8, 7.3 Hz, 2H), 4.75 (br s, 2H), 2.95 (br s, 1H), 1.85-1.78 (m, 1H), 0.94-0.88 (m, 2H), 0.62-0.57 (m, 2H).

### Synthesis of 2-((6-cyclopropyl-8-(3-hydroxyoxetan-3-yl)imidazo[1,2-a]pyridin-2-yl)methyl)isoindoline-1,3-dione

A mixture of 3-(2-amino-5-cyclopropylpyridin-3-yl)oxetan-3-ol (210 mg, 1.0 mmol) and 2-(3-bromo-2-oxopropyl)isoindoline-1,3-dione (280 mg, 1.0 mmol) in 1,4-dioxane (4.0 mL) was stirred at 80° C. for 18 h. The mixture was cooled to room temperature and diluted with EtOAc and K₂CO₃ (10% aq·). The mixture was separated and the aqueous layer was further extracted with EtOAc. The combined organic layers were washed with brine (sat·aq·), dried over MgSO₄, filtered and concentrated in vacuo. The residue was purified by column chromatography on silica gel, eluting with 1-6% MeOH in DCM to give the title compound (210 mg, 54%) as a light yellow solid. ESI-MS [M+H]+: 390.2, ¹H NMR (400 MHz, CDCl₃) δ 7.88-7.85 (m, 2H), 7.76-7.75 (m, 1H), 7.74-7.71 (m, 2H), 7.47 (s, 1H), 7.36 (s, 1H), 7.24 (d, J=1.6 Hz, 1H), 5.02-4.99 (m, 4H), 4.77 (d, J=7.3 Hz, 2H), 2.01-1.88 (m, 1H), 1.01-0.95 (m, 2H), 0.70-0.65 (m, 2H).

### Synthesis of 3-(2-(aminomethyl)-6-cyclopropylimidazo[1,2-a]pyridin-8-yl)oxetan-3-ol

A mixture of 2-((6-cyclopropyl-8-(3-hydroxyoxetan-3-yl)imidazo[1,2-a]pyridin-2-yl)methyl)isoindoline-1,3-dione (120 mg, 0.31 mmol) and hydrazine hydrate (87 μL, 1.8 mmol) in EtOH (3.0 mL) was heated at reflux for 1 h. The mixture was cooled to room temperature, filtered and concentrated in vacuo. The residue was redissolved in MeOH, filtered and concentrated in vacuo. The residue was purified by column chromatography on reverse phase silica gel, eluting with 30% MeCN in water (0.1% NH₅CO₃) to give the title compound (0.029 g, 36%) as an off-white solid. ¹H NMR (400 MHz, MeOD) δ, 8.19 (s, 1H), 7.84 (s, 1H), 7.21 (s, 1H), 5.37 (d, J=6.8 Hz, 2H), 4.25 (s, 2H), 3.35 (s, 2H), 2.04-1.94 (m, 1H), 1.03-0.96 (m, 2H), 0.76-0.71 (m, 2H), exchangeable OH and NH not observed.

### Synthesis of (6-cyclopropyl-8-(3-fluorooxetan-3-yl)imidazo[1,2-a]pyridin-2-yl)methanamine

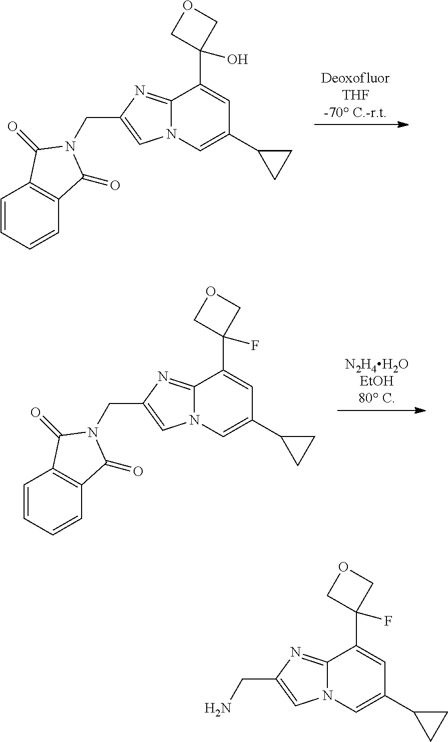 [SMILES: O=C1c2ccccc2C(=O)N1Cc1cn2cc(C3CC3)cc(C3(O)COC3)c2n1 ; "DeoxofluorTHF-70° C.-r.t." ; O=C1c2ccccc2C(=O)N1Cc1cn2cc(C3CC3)cc(C3(F)COC3)c2n1 ; "N2H4·H2OEtOH80° C." ; NCc1cn2cc(C3CC3)cc(C3(F)COC3)c2n1]

### Synthesis of 2-((6-cyclopropyl-8-(3-fluorooxetan-3-yl)imidazo[1,2-a]pyridin-2-yl)methyl)isoindoline-1,3-dione

Deoxofluor® (50% in THF, 0.6 mL, 1.6 mmol) was added to a solution of 2-((6-cyclopropyl-8-(3-hydroxyoxetan-3-yl)imidazo[1,2-a]pyridin-2-yl)methyl)isoindoline-1,3-dione (420 mg, 1.1 mmol) in DCM (20 mL) at −70° C. The mixture warmed to 0° C. and stirred for 3 h. Further Deoxofluor® (50% in THF, 0.3 mL, 0.82 mmol) was added and the mixture was stirred at 0° C. for a further 2 h. The mixture was warmed to room temperature and stirred for 18 h. The mixture was cooled to 0° C. and NaHCO₃ (sat·aq·, 15 mL) was added. The mixture was separated and the organic layer was dried over MgSO₄, filtered and concentrated in vacuo. The residue was purified by column chromatography on silica gel, eluting with 20-60% EtOAc in cyclohexane to give the title compound (190 mg, 44%) as a colourless solid. ESI-MS [M+H]+: 392.1, ¹H NMR (400 MHz, CDCl₃) δ 7.91-7.86 (m, 2H), 7.79 (s, 1H), 7.75-7.70 (m, 2H), 7.43 (s, 1H), 6.95 (s, 1H), 5.53-5.41 (m, 2H), 5.08 (s, 2H), 5.06-4.95 (m, 2H), 1.90-1.82 (m, 1H), 0.98-0.90 (m, 2H), 0.66-0.60 (m, 2H).

### Synthesis of (6-cyclopropyl-8-(3-fluorooxetan-3-yl)imidazo[1,2-a]pyridin-2-yl)methanamine

A mixture of 2-((6-cyclopropyl-8-(3-fluorooxetan-3-yl)imidazo[1,2-a]pyridin-2-yl)methyl)isoindoline-1,3-dione (180 mg, 0.46 mmol) in EtOH (5 mL) and hydrazine monohydrate (67 μL, 1.4 mmol) was stirred at reflux for 1 h. The mixture was cooled and concentrated in vacuo. The residue was dissolved in a mixture of DCM/MeOH (9:1, 8 mL), filtered and concentrated in vacuo to give the title compound (110 mg, 95%) as a viscous pale brown oil. ESI-MS [M+H]+: 262.0, ¹H NMR (400 MHz, CDCl₃) δ 7.88 (s, 1H), 7.44 (s, 1H), 6.97 (s, 1H), 5.56-5.46 (m, 2H), 5.14-5.04 (m, 2H), 4.02 (s, 2H), 1.01-0.93 (m, 2H), 0.70-0.64 (m, 2H), cyclopropyl CH signal obscured by water signal.

### Synthesis of tert-butyl 3-(2-(aminomethyl)-6-cyclopropylimidazo[1,2-a]pyridin-8-yl)-3-fluoroazetidine-1-carboxylate

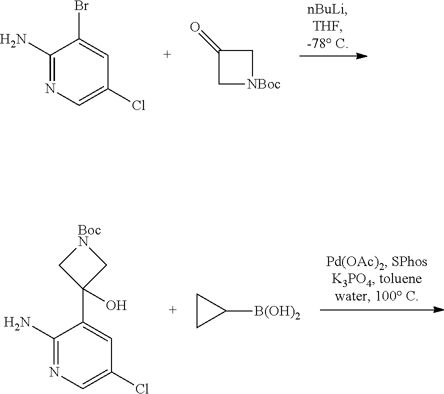 [SMILES: Nc1ncc(Cl)cc1Br ; CC(C)(C)OC(=O)N1CC(=O)C1 ; "nBuLi,THF,-78° C." ; CC(C)(C)OC(=O)N1CC(O)(c2cc(Cl)cnc2N)C1 ; OB(O)C1CC1 ; "Pd(OAc)2, SPhosK3PO4, toluenewater, 100° C."]

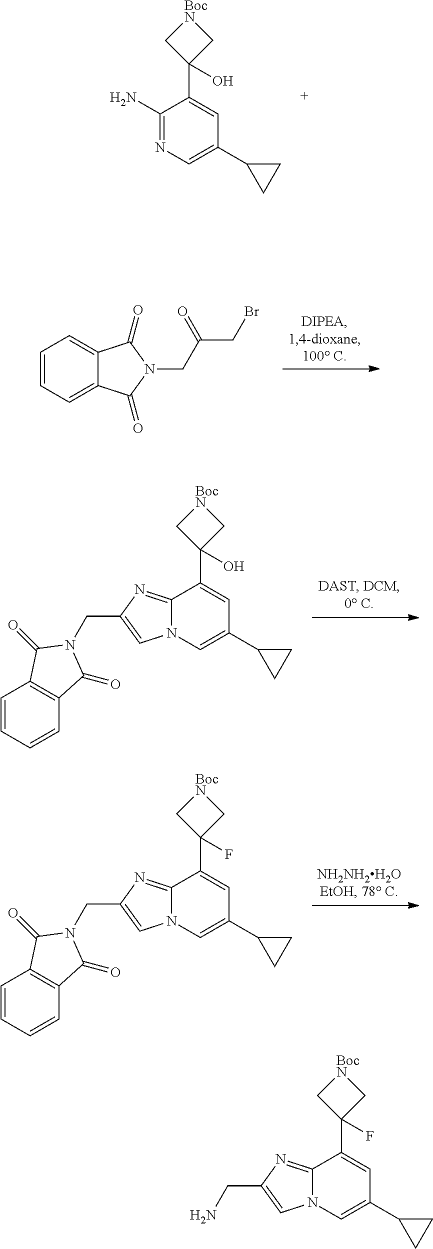

### Synthesis of tert-butyl 3-(6-cyclopropyl-2-((1,3-dioxoisoindolin-2-yl)methyl)imidazo[1,2-a]pyridin-8-yl)-3-hydroxyazetidine-1-carboxylate

Using a similar procedure to that for 2-((6-cyclopropyl-8-(3-hydroxyoxetan-3-yl)imidazo[1,2-a]pyridin-2-yl)methyl)isoindoline-1,3-dione, tert-butyl 3-(6-cyclopropyl-2-((1,3-dioxoisoindolin-2-yl)methyl)imidazo[1,2-a]pyridin-8-yl)-3-hydroxyazetidine-1-carboxylate (512 mg) was synthesised from 3-bromo-5-chloropyridin-2-amine (2.0 g, 9.64 mmol) and tert-butyl 3-oxoazetidine-1-carboxylate (6.6 g, 38.56 mmol). ESI-MS (M+H)+: 489.2, ¹H NMR (400 MHz, CDCl₃) δ 7.88-7.86 (m, 2H), 7.74-7.71 (m, 2H), 7.46 (s, 1H), 7.16 (s, 1H), 6.99 (d, J=1.5 Hz, 1H), 5.01 (s, 2H), 4.26-4.22 (m, 2H), 4.19-4.12 (m, 2H), 1.92-1.85 (m, 1H), 1.46 (s, 9H), 1.00-0.94 (m, 2H), 0.68-0.63 (m, 2H).

### Synthesis of tert-butyl 3-(6-cyclopropyl-2-((1,3-dioxoisoindolin-2-yl)methyl)imidazo[1,2-a]pyridin-8-yl)-3-fluoroazetidine-1-carboxylate

DAST (0.20 mL, 1.54 mmol) was added dropwise to a stirred solution of tert-butyl 3-(6-cyclopropyl-2-((1,3-dioxoisoindolin-2-yl)methyl)imidazo[1,2-a]pyridin-8-yl)-3-hydroxyazetidine-1-carboxylate (250 mg, 0.512 mmol) in DCM (20 mL) at 0° C. The reaction was stirred at 0° C. for 30 min. The reaction was quenched with NaHCO₃ solution (sat·aq·, 20 mL) and extracted with DCM (2×20 mL), the organics were combined, dried (MgSO₄) and concentrated in vacuo. The residue was purified by silica gel chromatography, eluting with a gradient of 0-100% EtOAc in cyclohexane to give the title compound (160 mg, 64%). ESI-MS (M+H)+: 491.2, ¹H NMR (400 MHz, CDCl₃) δ 7.90-7.87 (m, 2H), 7.76-7.72 (m, 3H), 7.38 (s, 1H), 6.97 (s, 1H), 5.05 (s, 2H), 4.89-4.78 (m, 2H), 4.30 (dd, J=10.2, 22.9 Hz, 2H), 1.89-1.81 (m, 1H), 1.52 (s, 9H), 0.97-0.91 (m, 2H), 0.65-0.60 (m, 2H).

### Synthesis of tert-butyl 3-(2-(aminomethyl)-6-cyclopropylimidazo[1,2-a]pyridin-8-yl)-3-fluoroazetidine-1-carboxylate

Using a similar procedure to that for 3-(2-(aminomethyl)-6-cyclopropylimidazo[1,2-a]pyridin-8-yl)oxetan-3-ol, tert-butyl 3-(2-(aminomethyl)-6-cyclopropylimidazo[1,2-a]pyridin-8-yl)-3-fluoroazetidine-1-carboxylate (110 mg, 93%) was synthesised from tert-butyl 3-(6-cyclopropyl-2-((1,3-dioxoisoindolin-2-yl)methyl)imidazo[1,2-a]pyridin-8-yl)-3-fluoroazetidine-1-carboxylate (160 mg, 0.326 mmol) using hydrazine hydrate. ESI-MS (M+H)+: 361.1, ¹H NMR (400 MHz, CDCl₃) δ 7.85 (s, 1H), 7.40 (s, 1H), 6.98 (d, J=1.4 Hz, 1H), 4.87 (dd, J=10.4, 22.8 Hz, 2H), 4.33 (dd, J=10.2, 22.6 Hz, 2H), 3.97 (s, 2H), 1.93-1.85 (m, 1H), 1.50 (s, 9H), 0.99-0.93 (m, 2H), 0.70-0.65 (m, 2H). Two exchangeable protons (NH₂) not visible

### Synthesis of (6-cyclopropyl-8-(3-fluoro-1-methylazetidin-3-yl)imidazo[1,2-a]pyridin-2-yl)methanamine

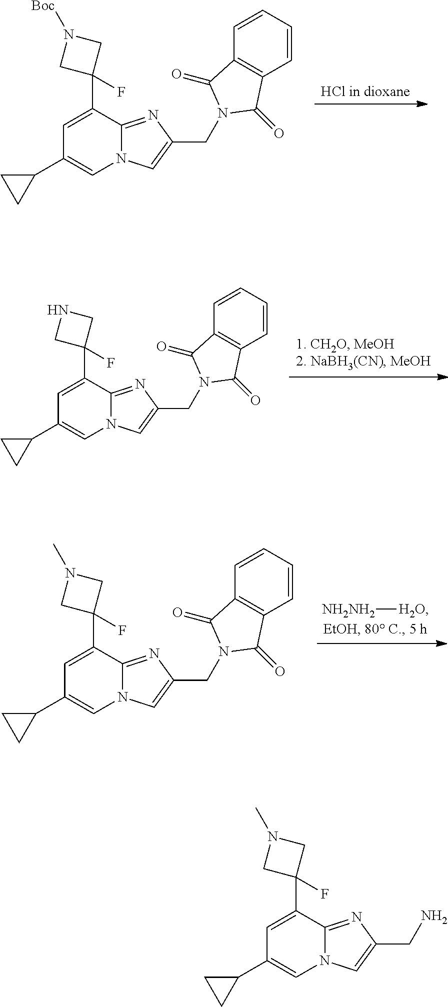

### Synthesis of 2-((6-cyclopropyl-8-(3-fluoroazetidin-3-yl)imidazo[1,2-a]pyridin-2-yl)methyl)isoindoline-1,3-dione

To a solution of tert-butyl 3-(6-cyclopropyl-2-((1,3-dioxoisoindolin-2-yl)methyl)imidazo[1,2-a]pyridin-8-yl)-3-fluoroazetidine-1-carboxylate (110 mg, 0.22 mmol) in MeOH (10 mL) was added HCl (4M solution in dioxane, 5 mL). The resulting mixture was stirred at room temperature for 1 h and concentrated in vacuo to give 2-((6-cyclopropyl-8-(3-fluoroazetidin-3-yl)imidazo[1,2-a]pyridin-2-yl)methyl)isoindoline-1,3-dione as a hydrochloric acid salt (90 mg, 96%) as a yellow oil, which was used in next step without purification. ESI-MS [M+H]+: 391.1.

### Synthesis of 2-((6-cyclopropyl-8-(3-fluoro-1-methylazetidin-3-yl)imidazo[1,2-a]pyridin-2-yl)methyl)isoindoline-1,3-dione

To a solution of 2-((6-cyclopropyl-8-(3-fluoroazetidin-3-yl)imidazo[1,2-a]pyridin-2-yl)methyl)isoindoline-1,3-dione hydrochloride (89.5 mg, 0.21 mmol), paraformaldehyde (63 mg, 2.1 mmol) and NaBH₃CN ((93 mg, 1.47 mmol) in MeOH (10 mL) was stirred at room temperature for 4 h. The mixture was then quenched with water (20 mL) and extracted with EtOAc (3×20 mL). The combined organic layers were washed with brine (50 mL), dried over anhydrous Na₂SO₄ and concentrated in vacuo to give the crude, which was purified by silica gel chromatography (DCM/MeOH=20/1) to give 2-((6-cyclopropyl-8-(3-fluoro-1-methylazetidin-3-yl)imidazo[1,2-a]pyridin-2-yl)methyl)isoindoline-1,3-dione (51 mg, 60%) as a yellow oil. ESI-MS [M+H]+: 405.2

### Synthesis of (6-cyclopropyl-8-(3-fluoro-1-methylazetidin-3-yl)imidazo[1,2-a]pyridin-2-yl)methanamine

To a solution of 2-((6-cyclopropyl-8-(3-fluoro-1-methylazetidin-3-yl)imidazo[1,2-a]pyridin-2-yl)methyl)isoindoline-1,3-dione (50 mg, 0.12 mmol) in EtOH (10 mL) was added N₂H₄⁻H₂O (1 mL). After stirring at 80° C. for 5 h, the reaction mixture was filtered and the filtrate was concentrated in vacuo to give (6-cyclopropyl-8-(3-fluoro-1-methylazetidin-3-yl)imidazo[1,2-a]pyridin-2-yl)methanamine (30 mg, 91%) as a white solid, which was used in next step without purification. ESI-MS [M+H]⁺: 275.1.

### Synthesis of tert-butyl 3-(2-(aminomethyl)-6-cyclopropylimidazo[1,2-a]pyridin-8-yl)-3-hydroxyazetidine-1-carboxylate

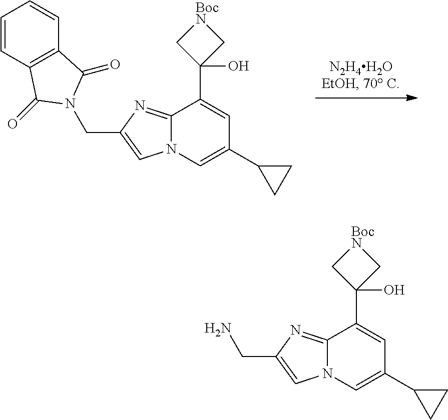 [SMILES: CC(C)(C)OC(=O)N1CC(O)(c2cc(C3CC3)cn3cc(CN4C(=O)c5ccccc5C4=O)nc23)C1 ; "N2H4·H2OEtOH, 70° C." ; CC(C)(C)OC(=O)N1CC(O)(c2cc(C3CC3)cn3cc(CN)nc23)C1]

Using a similar procedure to that for 3-(2-(aminomethyl)-6-cyclopropylimidazo[1,2-a]pyridin-8-yl)oxetan-3-ol, tert-butyl 3-(2-(aminomethyl)-6-cyclopropylimidazo[1,2-a]pyridin-8-yl)-3-hydroxyazetidine-1-carboxylate (180 mg, 99%) was synthesised from tert-butyl 3-(6-cyclopropyl-2-((1,3-dioxoisoindolin-2-yl)methyl)imidazo[1,2-a]pyridin-8-yl)-3-hydroxyazetidine-1-carboxylate (250 mg, 0.51 mmol) using hydrazine hydrate. ¹H NMR (400 MHz, CDCl₃) δ 7.79 (s, 1H), 7.38 (s, 1H), 7.01-6.99 (m, 1H), 4.29 (d, J=9.3 Hz, 2H), 4.24-4.19 (m, 2H), 3.97 (d, J=0.6 Hz, 2H), 1.97-1.88 (m, 1H), 1.47 (s, 9H), 1.02-0.96 (m, 2H), 0.71-0.66 (m, 2H). NH₂ and OH not observed

### Synthesis of 3-(2-(aminomethyl)-6-cyclopropylimidazo[1,2-a]pyridin-8-yl)-1-methylazetidin-3-ol

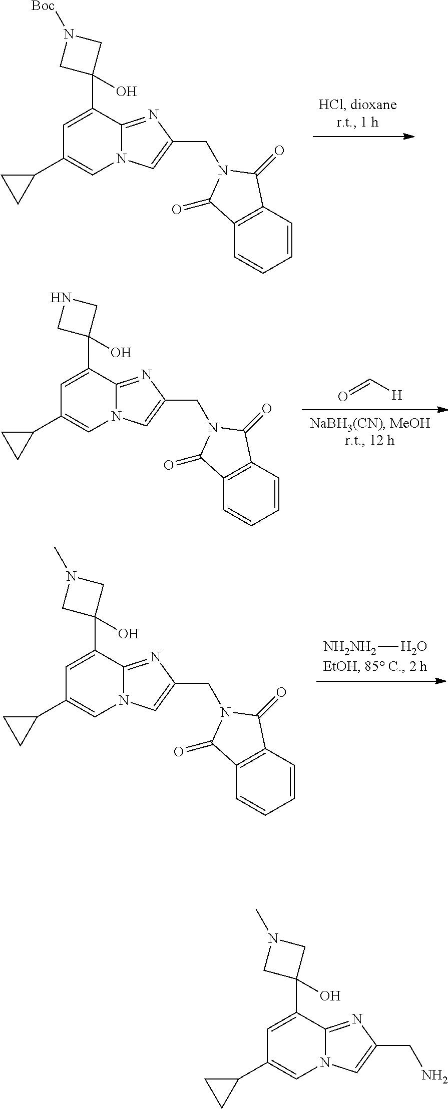

### Synthesis of 2-((6-cyclopropyl-8-(3-hydroxyazetidin-3-yl)imidazo[1,2-a]pyridin-2-yl)methyl)isoindoline-1,3-dione

To a solution of tert-butyl 3-(6-cyclopropyl-2-((1,3-dioxoisoindolin-2-yl)methyl)imidazo[1,2-a]pyridin-8-yl)-3-hydroxyazetidine-1-carboxylate (3.1 g, 6.35 mmol) in dioxane (20 mL) was added HCl (8 mL, 4 M solution in dioxane, 32 mmol). The resulting mixture was stirred at room temperature for 1 h. The solution was concentrated in vacuo to give the residue, which was washed with saturated aqueous NaHCO₃ (50 mL), extracted with EtOAc (3×70 mL). The combined organic layers were washed with brine (70 mL), dried over Na₂SO₄, concentrated in vacuo to give the crude product, which was purified with silica gel chromatography (eluent: DMC/MeOH=15/1) to give 2-((6-cyclopropyl-8-(3-hydroxyazetidin-3-yl)imidazo[1,2-a]pyridin-2-yl)methyl)isoindoline-1,3-dione (1.5 g, 61%) as a yellow solid. ESI-MS [M+H]⁺: 389.1.

### Synthesis of 2-((6-cyclopropyl-8-(3-hydroxy-1-methylazetidin-3-yl)imidazo[1,2-a]pyridin-2-yl)methyl)isoindoline-1,3-dione

To a solution of 2-((6-cyclopropyl-8-(3-hydroxyazetidin-3-yl)imidazo[1,2-a]pyridin-2-yl)methyl)isoindoline-1,3-dione (1.5 g, 3.86 mmol) in MeOH (30 mL) were added paraformaldehyde (232 mg, 7.72 mmol) and NaBH₃(CN) (487 mg, 7.72 mmol). The resulting mixture was stirred at room temperature for 12 h. Then water (50 mL) was added and the mixture extracted with EtOAc (3×70 mL). The combined organic layers were washed with brine (50 mL), dried over Na₂SO₄ and concentrated in vacuo give the crude product, which was purified with silica gel chromatography (eluent: DCM/MeOH=20/1) to give 2-((6-cyclopropyl-8-(3-hydroxy-1-methylazetidin-3-yl)imidazo[1,2-a]pyridin-2-yl)methyl)isoindoline-1,3-dione (1.1 g, 71%) as a yellow solid. ESI-MS [M+H]⁺: 403.2.

### Synthesis of 3-(2-(aminomethyl)-6-cyclopropylimidazo[1,2-a]pyridin-8-yl)-1-methylazetidin-3-ol

A solution of 2-((6-cyclopropyl-8-(3-hydroxy-1-methylazetidin-3-yl)imidazo[1,2-a]pyridin-2-yl)methyl)isoindoline-1,3-dione (1.1 g, 2.74 mmol) in NH₂NH₂—H₂O (2 mL) in EtOH (20 mL) was stirred at 85° C. for 2 h. After cooling to the room temperature, the white solid was precipitated and filtered. The filtrate was concentrated in vacuo to give 3-(2-(aminomethyl)-6-cyclopropylimidazo[1,2-a]pyridin-8-yl)-1-methylazetidin-3-ol (700 mg crude), which was used into next step without further purification. ESI-MS [M+H]⁺: 273.2.

### Synthesis of tert-butyl 3-((2-(aminomethyl)-6-cyclopropylimidazo[1,2-a]pyridin-8-yl)(hydroxy)methyl)azetidine-1-carboxylate

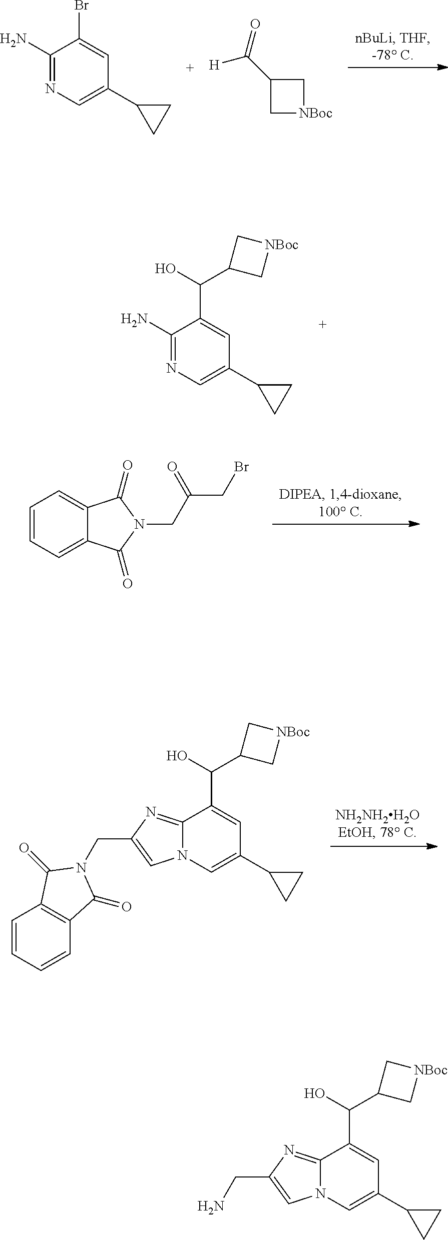

Using a similar procedure to that for 3-(2-(aminomethyl)-6-cyclopropylimidazo[1,2-a]pyridin-8-yl)oxetan-3-ol, tert-butyl 3-((2-(aminomethyl)-6-cyclopropylimidazo[1,2-a]pyridin-8-yl)(hydroxy)methyl)azetidine-1-carboxylate (80 mg) was synthesised from 3-bromo-5-cyclopropylpyridin-2-amine (633 mg, 2.97 mmol) and tert-butyl 3-formylazetidine-1-carboxylate (1.10 g, 5.94 mmol). ESI-MS (M+H)+: 373.2

### Synthesis of 4-(2-(aminomethyl)-6-cyclopropylimidazo[1,2-a]pyridin-8-yl)-1-methylpiperidin-4-ol

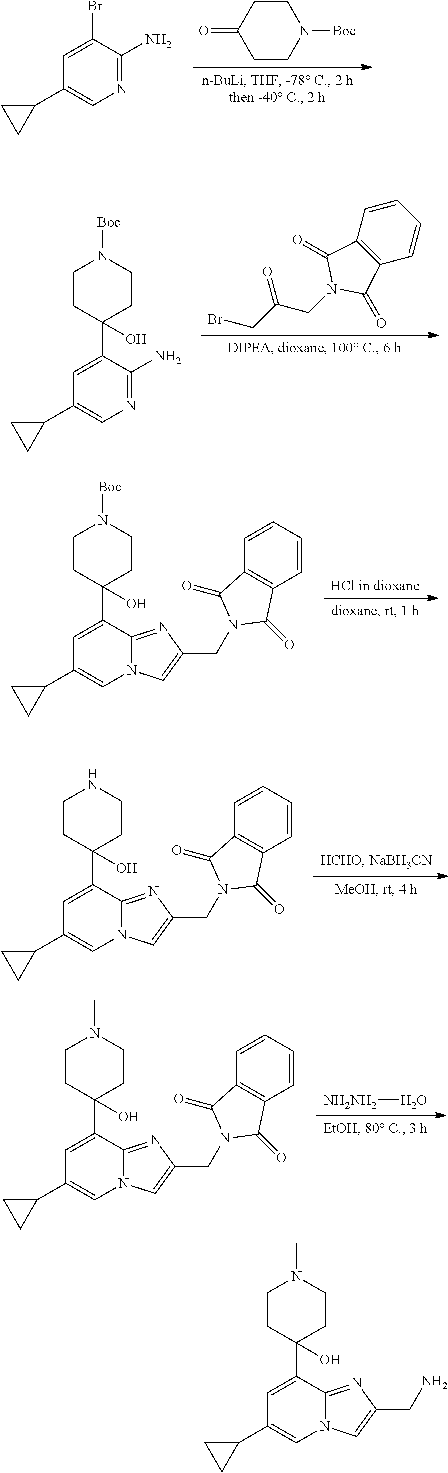

### Synthesis of tert-butyl 4-(6-cyclopropyl-2-((1,3-dioxoisoindolin-2-yl)methyl)imidazo[1,2-a]pyridin-8-yl)-4-hydroxypiperidine-1-carboxylate

Using a similar procedure to that for 3-(2-(aminomethyl)-6-cyclopropylimidazo[1,2-a]pyridin-8-yl)oxetan-3-ol, tert-butyl 4-(6-cyclopropyl-2-((1,3-dioxoisoindolin-2-yl)methyl)imidazo[1,2-a]pyridin-8-yl)-4-hydroxypiperidine-1-carboxylate (937 mg, 76%) was synthesised from 3-bromo-5-cyclopropylpyridin-2-amine (500 mg, 2.35 mmol) and tert-butyl 4-oxopiperidine-1-carboxylate (1.9 g, 9.54 mmol). ESI-MS [M+H]+ 517.2

### Synthesis of 2-((6-cyclopropyl-8-(4-hydroxypiperidin-4-yl)imidazo[1,2-a]pyridin-2-yl)methyl)isoindoline-1,3-dione hydrochloride

To a solution of tert-butyl 4-(6-cyclopropyl-2-((1,3-dioxoisoindolin-2-yl)methyl)imidazo[1,2-a]pyridin-8-yl)-4-hydroxypiperidine-1-carboxylate (937 mg, 1.82 mmol) in dioxane (3 mL) was added HCl (4 M in dioxane, 3 mL) at room temperature and stirred for 1 h. The reaction mixture was concentrated to give 2-((6-cyclopropyl-8-(4-hydroxypiperidin-4-yl)imidazo[1,2-a]pyridin-2-yl)methyl)isoindoline-1,3-dione hydrochloride (866 mg, crude) as a white solid, which was used in next step without purification. ESI-MS [M+H]⁺: 417.2.

### Synthesis of 2-((6-cyclopropyl-8-(4-hydroxy-1-methylpiperidin-4-yl)imidazo[1,2-a]pyridin-2-yl)methyl) isoindoline-1,3-dione

A mixture of 2-((6-cyclopropyl-8-(4-hydroxypiperidin-4-yl)imidazo[1,2-a]pyridin-2-yl)methyl) isoindoline-1,3-dione hydrochloride (866 mg, 2.08 mmol) and HCHO (779 mg, 9.60 mmol) in MeOH (20 mL) was added NaBH₃CN (302 mg, 4.80 mmol) at room temperature. The mixture was stirred at rt for 4 h. H₂O (30 mL) was added and extracted with EtOAc (3×30 mL). The combined organic layers were washed with brine (30 mL), dried over Na₂SO₄ and concentrated in vacuo give the crude product, which was purified by silica gel chromatography (eluent: DCM/MeOH=9/1) to give 2-((6-cyclopropyl-8-(4-hydroxy-1-methylpiperidin-4-yl)imidazo[1,2-a]pyridin-2-yl)methyl)isoindoline-1,3-dione (822 mg, 92%) as a yellow oil. ESI-MS [M+H]⁺: 431.2.

### Synthesis of 4-(2-(aminomethyl)-6-cyclopropylimidazo[1,2-a]pyridin-8-yl)-1-methylpiperidin-4-ol

A solution of 2-((6-cyclopropyl-8-(4-hydroxy-1-methylpiperidin-4-yl)imidazo[1,2-a]pyridin-2-yl)methyl)isoindoline-1,3-dione (400 mg, 0.93 mmol) in EtOH (15 mL) was added NH₂NH₂—H₂O (0.5 mL) at room temperature. Then the mixture was stirred at 80° C. for 3 h and then cooled to room temperature. The reaction mixture was filtered and the filtrate was concentrated in vacuo to give 4-(2-(aminomethyl)-6-cyclopropylimidazo[1,2-a]pyridin-8-yl)-1-methylpiperidin-4-ol (407 mg, crude) as a yellow solid, which was used directly into next step without further purification. ESI-MS [M+H]⁺: 301.2.

### Synthesis of (6-cyclopropyl-8-(4-fluoro-1-methylpiperidin-4-yl)imidazo[1,2-a]pyridin-2-yl)methanamine

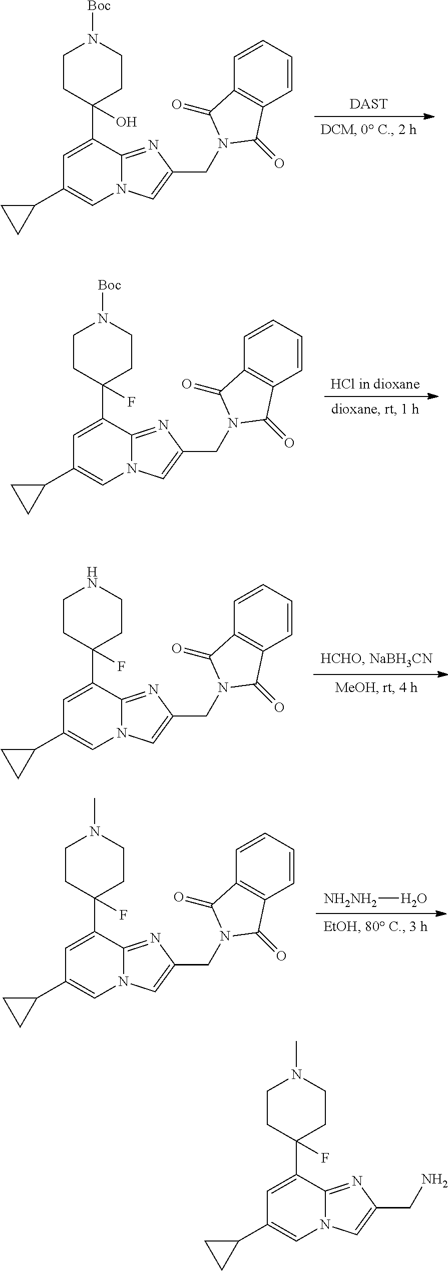

### Synthesis of tert-butyl 4-(6-cyclopropyl-2-((1,3-dioxoisoindolin-2-yl)methyl)imidazo[1,2-a]pyridin-8-yl)-4-fluoropiperidine-1-carboxylate

To a solution of tert-butyl 4-(6-cyclopropyl-2-((1,3-dioxoisoindolin-2-yl)methyl)imidazo[1,2-a]pyridin-8-yl)-4-hydroxy piperidine-1-carboxylate (517 mg, 1.0 mmol) in DCM (10 mL) was added a solution of DAST (177 mg, 1.1 mmol) in DCM (5 mL) dropwise at 0° C. After stirring at 0° C. for 2 h under N₂, the mixture was quenched with water (20 mL) and extracted by DCM (3×20 mL). The combined organic layers were washed with brine (20 mL) and concentrated in vacuo to give the crude product, which was purified by silica gel chromatography (eluent: PE/EtOAc=2/1) to give tert-butyl 4-(6-cyclopropyl-2-((1,3-dioxoisoindolin-2-yl)methyl)imidazo[1,2-a]pyridin-8-yl)-4-fluoropiperidine-1-carboxylate (447 mg, 86%) as a yellow solid. ESI-MS [M+H]⁺: 519.2.

### Synthesis of 2-((6-cyclopropyl-8-(4-fluoropiperidin-4-yl)imidazo[1,2-a]pyridin-2-yl)methyl)isoindoline-1,3-dione hydrochloride

To a solution of tert-butyl 4-(6-cyclopropyl-2-((1,3-dioxoisoindolin-2-yl)methyl)imidazo[1,2-a] pyridine-8-yl)-4-fluoropiperidine-1-carboxylate (347 mg, 0.67 mmol) in dioxane (2 mL) was added HCl (4 M in dioxane, 2 mL) at room temperature and stirred at rt for 1 h. The reaction mixture was concentrated in vacuo to give 2-((6-cyclopropyl-8-(4-fluoropiperidin-4-yl)imidazo[1,2-a]pyridin-2-yl)methyl)isoindoline-1,3-dione as the hydrochloric acid salt (300 mg, crude) as a white solid which was used in next step without purification. ESI-MS [M+H]⁺: 419.2.

### Synthesis of 2-((6-cyclopropyl-8-(4-fluoro-1-methylpiperidin-4-yl)imidazo[1,2-a]pyridin-2-yl)methyl)isoindoline-1,3-dione

To a mixture of 2-((6-cyclopropyl-8-(4-fluoropiperidin-4-yl)imidazo[1,2-a]pyridin-2-yl)methyl)isoindoline-1,3-dione hydrochloride (200 mg, 0.44 mmol) and paraformaldehyde (66 mg, 2.2 mmol) in MeOH (5 mL) was added NaBH₃CN (70 mg, 1.1 mmol) at room temperature. After stirring for 4 h, water (30 mL) was added and extracted with EtOAc (3×50 mL). The combined organic layers were washed with brine (30 mL), dried over Na₂SO₄ and concentrated in vacuo to give the crude product, which was purified by preparative TLC (eluent: DCM/MeOH=10/1) to give 2-((6-cyclopropyl-8-(4-fluoro-1-methylpiperidin-4-yl)imidazo[1,2-a]pyridin-2-yl)methyl)isoindoline-1,3-dione (160 mg, 84%) as a white solid. ESI-MS [M+H]⁺: 433.2.

### Synthesis of (6-cyclopropyl-8-(4-fluoro-1-methylpiperidin-4-yl)imidazo[1,2-a]pyridin-2-yl)methanamine

To a mixture of 2-((6-cyclopropyl-8-(4-fluoro-1-methylpiperidin-4-yl)imidazo[1,2-a]pyridin-2-yl) methyl)isoindoline-1,3-dione (312 mg, 0.72 mmol) in EtOH (20 mL) was added NH₂NH₂—H₂O (225 mg, 3.6 mmol) at room temperature. After stirring at 80° C. for 3 h, the reaction mixture was filtered and the filtrate was concentrated to give (6-cyclopropyl-8-(4-fluoro-1-methylpiperidin-4-yl)imidazo[1,2-a]pyridin-2-yl)methanamine (200 mg, crude) as a yellow solid, which was used directly in the next step without further purification. ESI-MS [M+H]⁺: 303.2.

### Synthesis of tert-butyl 4-(2-(aminomethyl)-6-cyclopropylimidazo[1,2-a]pyridin-8-yl)piperazine-1-carboxylate

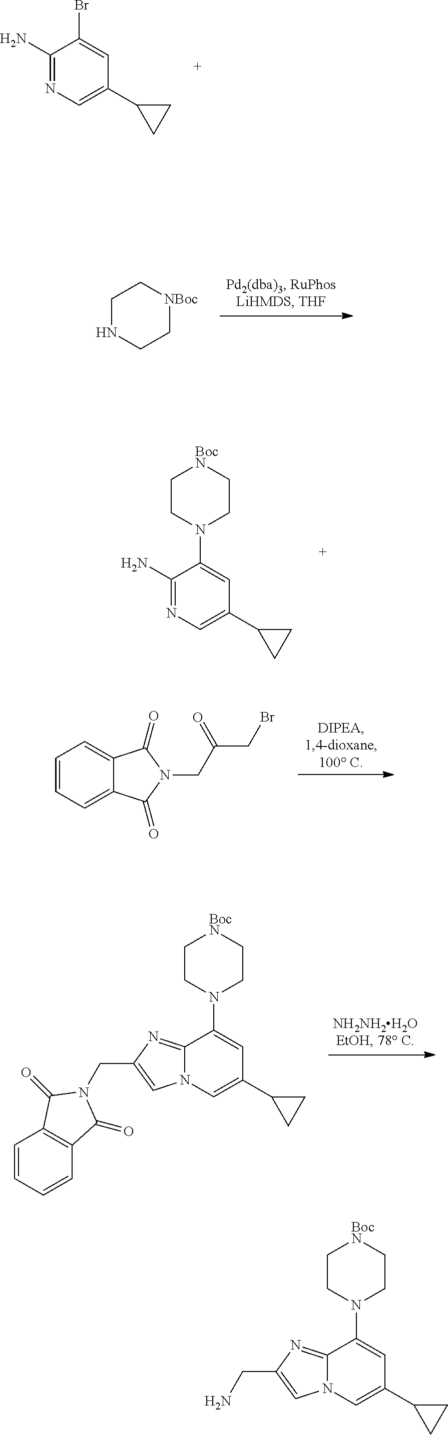

### Synthesis of tert-Butyl 4-(2-amino-5-cyclopropylpyridin-3-yl)piperazine-1-carboxylate

A mixture of 3-bromo-5-cyclopropylpyridin-2-amine (1.00 g, 4.69 mmol), 1-Boc-piperazine (1.31 g, 7.04 mmol), Pd₂(dba)₃ (0.43 g, 0.47 mmol), and RuPhos (0.44 g, 0.94 mmol) and degassed with N₂ for 10 min. THF (40 mL) and LiHMDS (1M in THF, 11.7 mL, 11.7 mmol) were added and the resulting reaction mixture was heated to 65° C. for 90 min. The reaction mixture was allowed to cool to room temperature and was quenched with NH₄Cl (sat·aq·, 100 mL) and extracted with EtOAc (3×75 mL). The combined organics were dried over MgSO₄ and concentrated in vacuo. The residue was purified by silica gel chromatography, eluting with a gradient of 0-100% EtOAc in cyclohexane to give the title compound (0.940 g, 63%). ESI-MS (M+H)+: 319.3, ¹H NMR (400 MHz, CDCl₃): δ ppm, 7.67 (1H, d, J=2.0 Hz), 6.86 (1H, d, J=2.0 Hz), 4.55 (2H, br s), 3.56 (4H, br s), 2.84 (4H, t, J=4.7 Hz), 1.82-1.74 (1H, m), 1.49 (9H, s), 0.91-0.85 (2H, m), 0.60-0.55 (2H, m).

### Synthesis of tert-Butyl 4-(6-cyclopropyl-2-((1,3-dioxoisoindolin-2-yl)methyl)imidazo[1,2-a]pyridin-8-yl)piperazine-1-carboxylate

tert-Butyl 4-(2-amino-5-cyclopropylpyridin-3-yl)piperazine-1-carboxylate (940 mg, 2.95 mmol) and 2-(3-bromo-2-oxopropyl)isoindoline-1,3-dione (1.02 g, 3.25 mmol) were dissolved in 1,4-dioxane (90 mL) and DIPEA (0.77 mL, 4.43 mmol) was added. The reaction mixture was heated to 100° C. and stirred at this temperature for 4.5 h. The reaction mixture was cooled to room temperature and concentrated in vacuo. The residue was purified by silica gel chromatography, eluting with a gradient of 0-100% EtOAc in cyclohexane to give the title compound (1.38 g, 93%). ESI-MS (M+H)+: 502.2, ¹H NMR (400 MHz, CDCl₃): δ ppm, 7.87 (2H, dd, J=3.1, 5.5 Hz), 7.72 (2H, dd, J=3.1, 5.5 Hz), 7.44 (1H, dd, J=1.0, 1.0 Hz), 7.35 (1H, d, J=1.0 Hz), 6.14 (1H, d, J=1.0 Hz), 5.03 (2H, s), 3.62 (4H, t, J=5.1 Hz), 3.41 (4H, br s), 1.84-1.76 (1H, m), 1.49 (9H, s), 0.91-0.85 (2H, m), 0.62-0.57 (2H, m).

### Synthesis of tert-Butyl 4-(2-(aminomethyl)-6-cyclopropylimidazo[1,2-a]pyridin-8-yl)piperazine-1-carboxylate

Using a similar procedure to that for 3-(2-(aminomethyl)-6-cyclopropylimidazo[1,2-a]pyridin-8-yl)oxetan-3-ol, tert-butyl 4-(2-(aminomethyl)-6-cyclopropylimidazo[1,2-a]pyridin-8-yl)piperazine-1-carboxylate (1.14 g, 85%) was synthesised from tert-butyl 4-(6-cyclopropyl-2-((1,3-dioxoisoindolin-2-yl)methyl)imidazo[1,2-a]pyridin-8-yl)piperazine-1-carboxylate (1.38 g, 2.75 mmol) using hydrazine hydrate. ESI-MS (M+H)+: 372.3, ¹H NMR (400 MHz, CDCl₃): δ ppm, 7.52 (1H, s), 7.31 (1H, s), 6.19 (1H, d, J=1.6 Hz), 3.98 (2H, s), 3.69 (4H, t, J=4.9 Hz), 3.43 (4H, t, J=4.9 Hz), 1.86-1.79 (1H, m), 1.49 (9H, s), 0.94-0.89 (2H, m), 0.66-0.61 (2H, m).

Using a similar procedure to that used for tert-butyl 4-(2-(aminomethyl)-6-cyclopropylimidazo[1,2-a]pyridin-8-yl)piperazine-1-carboxylate, the compounds in Table A5 were prepared starting from 3-bromo-5-cyclopropylpyridin-2-amine and an appropriate coupling partner.

TABLE A5

| Structure | Coupling Partner | Analytical Data |
|---|---|---|
| 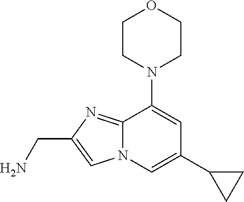 [SMILES: NCc1cn2cc(C3CC3)cc(N3CCOCC3)c2n1] | morpholine | used without further purification |
| (6-cyclopropyl-8-morpholinoimidazo[1,2-a] | | |
| pyridin-2-yl)methanamine | | |

### Synthesis of (6-cyclopropyl-8-(4-cyclopropylpiperazin-1-yl)imidazo[1,2-a]pyridin-2-yl)methanamine

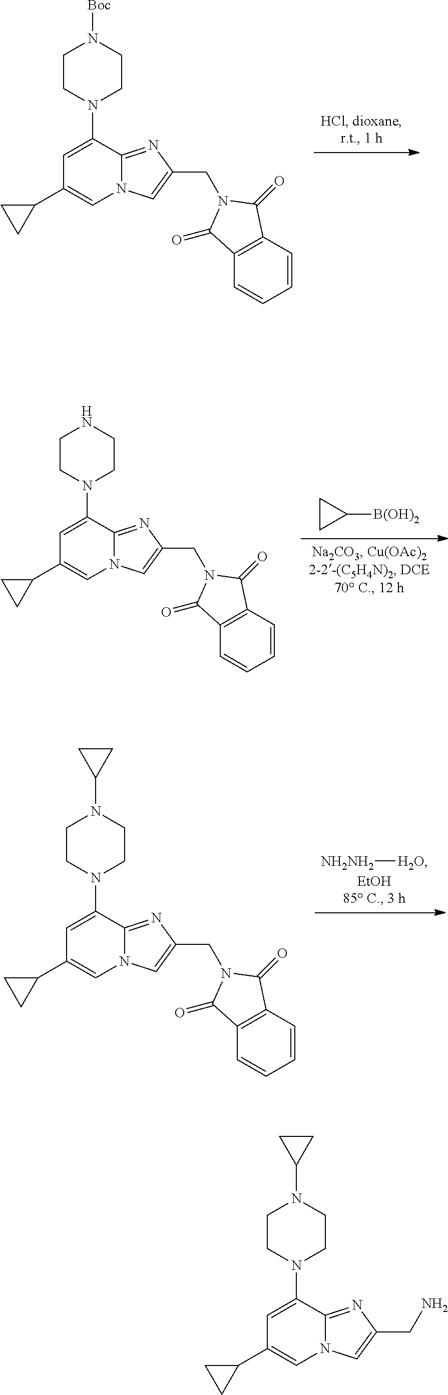

### Synthesis of 2-((6-cyclopropyl-8-(piperazin-1-yl)imidazo[1,2-a]pyridin-2-yl)methyl)isoindoline-1,3-dione

To a solution of tert-butyl 4-(6-cyclopropyl-2-((1,3-dioxoisoindolin-2-yl)methyl)imidazo[1,2-a]pyridin-8-yl)piperazine-1-carboxylate (7 g, 13.97 mmol) in dioxane (30 mL) was added HCl (13.75 mL, 4 M solution in dioxane, 55 mmol). The resulting mixture was stirred at room temperature for 1 h. The reaction was concentrated in vacuo to give the residue, which was washed with saturated aqueous NaHCO₃ (150 mL) and extracted with EtOAc (3×70 mL). The combined organic layers were washed with brine (70 mL), dried over Na₂SO₄, concentrated in vacuo to give the crude residue, which was purified with silica gel chromatography (eluent: DMC/MeOH=15/1) to give 2-((6-cyclopropyl-8-(piperazin-1-yl)imidazo[1,2-a]pyridin-2-yl)methyl)isoindoline-1,3-dione (4.5 g, 81.8%) as a yellow solid. ESI-MS [M+H]⁺: 402.1.

### Synthesis of 2-((6-cyclopropyl-8-(4-cyclopropylpiperazin-1-yl)imidazo[1,2-a]pyridin-2-yl)methyl)isoindoline-1,3-dione

To a mixture of 2-((6-cyclopropyl-8-(piperazin-1-yl)imidazo[1,2-a]pyridin-2-yl)methyl)isoindoline-1,3-dione (750 mg, 1.87 mmol), cyclopropylboronic acid (322 mg, 3.74 mmol) and Na₂CO₃ (396 mg, 3.74 mmol) in DCE (10 mL) were added Cu(OAc)₂ (338 mg, 1.87 mmol) and 2,2′-bipyridine (292 mg, 1.87 mmol) in DCE (10 mL). After stirring at 70° C. for 12 h, the reaction mixture was cooled to room temperature and filtered. The resulting filtrate was washed with water (30 mL) and extracted with DCM (30 mL×3). The combined organic layers were washed with brine (50 mL), dried over Na₂SO₄ then concentrated in vacuo to give the crude, which was purified by silica gel chromatography (eluant: DCM/MeOH=20/1) to give the product 2-((6-cyclopropyl-8-(4-cyclopropylpiperazin-1-yl)imidazo[1,2-a]pyridin-2-yl)methyl)isoindoline-1,3-dione (250 mg, 30%) as a yellow oil. ESI-MS [M+H]+: 442.2

### Synthesis of (6-cyclopropyl-8-(4-cyclopropylpiperazin-1-yl)imidazo[1,2-a]pyridin-2-yl)methanamine

A mixture of 2-((6-cyclopropyl-8-(4-cyclopropylpiperazin-1-yl)imidazo[1,2-a]pyridin-2-yl)methyl)isoindoline-1,3-dione (150 mg, 0.34 mmol) and NH₂NH₂—H₂O (170 mg, 3.40 mmol) in EtOH (20 mL) was stirred at 85° C. for 3 h. The reaction was cooled to room temperature and the mixture was filtered. The filtrate was concentrated in vacuo to give the crude product (6-cyclopropyl-8-(4-cyclopropylpiperazin-1-yl)imidazo[1,2-a]pyridin-2-yl)methanamine (120 mg, crude) as a yellow oil which was used in next step without purification. ESI-MS [M+H]+: 312.2

### Synthesis of 3-bromo-5-cyclopropylpyridin-2-amine

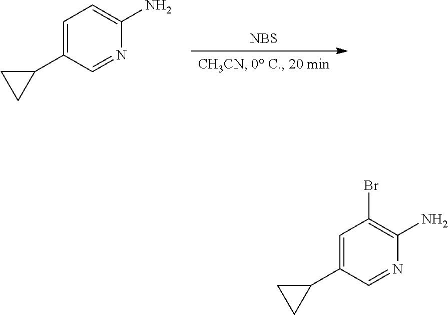 [SMILES: Nc1ccc(C2CC2)cn1 ; "NBS" ; "CH3CN, 0° C., 20 min" ; Nc1ncc(C2CC2)cc1Br]

To a stirred solution of 5-cyclopropylpyridin-2-amine (10 g, 74 mmol) in CH₃CN (200 mL) was added NBS (20 g, 111.9 mmol) at 0° C. and then the mixture was stirred at room temperature for 20 min. The reaction mixture was poured into water (250 mL) and extracted with EtOAc (250 mL×3). The combined organics were washed with brine (250 mL), dried over Na₂SO₄. The organic layers were concentrated in vacuo and purified by silica gel chromatography (eluent: PE:EA=10:1) to give 3-bromo-5-cyclopropylpyridin-2-amine (10 g, yield 62%) ESI-MS [M+H]+: 213.0

### Synthesis of 2-((8-bromo-6-cyclopropylimidazo[1,2-a]pyridin-2-yl)methyl)isoindoline-1,3-dione

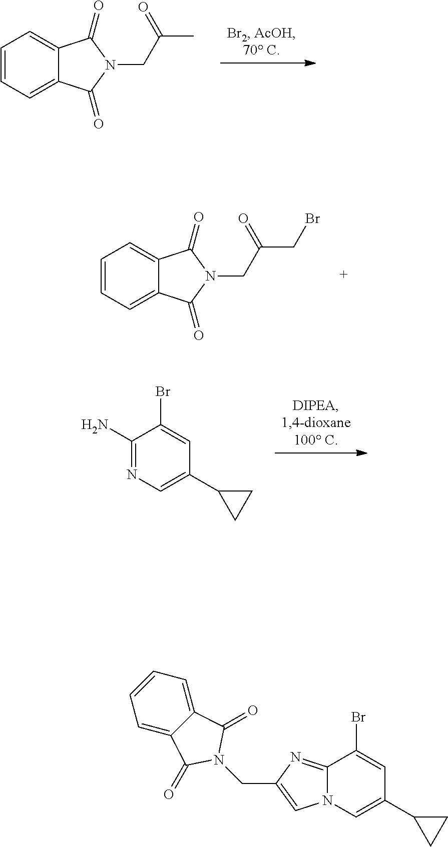 [SMILES: CC(=O)CN1C(=O)c2ccccc2C1=O ; "Br2, AcOH,70° C." ; O=C(CBr)CN1C(=O)c2ccccc2C1=O ; Nc1ncc(C2CC2)cc1Br ; "DIPEA,1,4-dioxane100° C." ; O=C1c2ccccc2C(=O)N1Cc1cn2cc(C3CC3)cc(Br)c2n1]

### Synthesis of 2-(3-bromo-2-oxopropyl)isoindoline-1,3-dione

A few drops of a solution of bromine (7.6 mL, 150 mmol) in AcOH (80 mL) was added to a stirred solution of 2-(2-oxopropyl)isoindoline-1,3-dione (20 g, 98 mmol) at 70° C. The reaction was stirred until the solution went colourless. The remainder of the bromine/AcOH solution was added dropwise over 2 h. The reaction mixture was stirred at 70° C. for a further 2 h. The mixture was cooled to room temperature and concentrated in vacuo. The residue was dissolved in DCM (300 mL) and washed with Na₂S₂O₃ solution (1.0 M, 75 mL) and Na₂CO₃ solution (10% aq·, 2×150 mL). The combined organics were dried over MgSO₄ and concentrated in vacuo. The residue was triturated with hot diethyl ether, filtered and dried to give the title compound (22.6 g, 81%) as a white solid. ¹H NMR (400 MHz, CDCl₃) δ 7.92-7.87 (m, 2H), 7.79-7.73 (m, 2H), 4.78 (s, 2H), 4.01 (s, 2H).

### Synthesis of 2-((8-bromo-6-cyclopropylimidazo[1,2-a]pyridin-2-yl)methyl)isoindoline-1,3-dione

A solution of 2-(3-bromo-2-oxopropyl)isoindoline-1,3-dione (12 g, 39 mmol), 3-bromo-5-cyclopropylpyridin-2-amine (7.5 g, 35 mmol) and DIPEA (9.2 mL, 53 mmol) in 1,4-dioxane (350 mL) was heated at 100° C. for 16 h. The mixture was cooled to room temperature and the volume was reduced by a half by concentrating in vacuo. The mixture was diluted with DCM (200 mL) and washed with NaHCO₃ solution (sat·aq·, 150 mL) and brine (150 mL). The organics were dried over MgSO₄ and concentrated in vacuo. The residue was purified by column chromatography on silica gel, eluting with a gradient of 0-100% EtOAc in cyclohexane to give the title compound (7.2 g, 52%). ESI-MS (M+H)+: 396.1, 398.1 ¹H NMR (400 MHz, DMSO) δ 8.35 (d, J=1.0 Hz, 1H), 7.99-7.94 (m, 2H), 7.94-7.91 (m, 3H), 7.39 (d, J=1.5 Hz, 1H), 4.94 (s, 2H), 2.02-1.94 (m, 1H), 0.96 (m, 2H), 0.76-0.71 (m, 2H).

### Synthesis of 2-((8-chloro-6-cyclopropylimidazo[1,2-a]pyridin-2-yl)methyl)isoindoline-1,3-dione

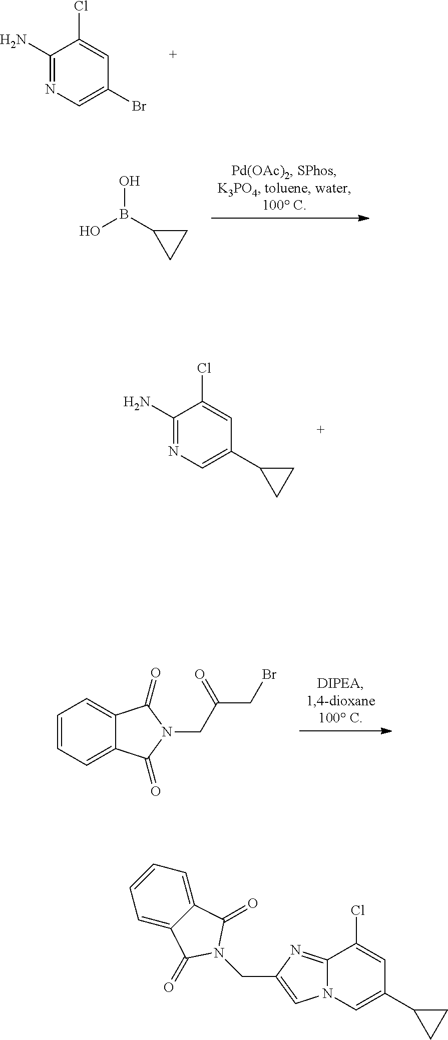

2-((8-chloro-6-cyclopropylimidazo[1,2-a]pyridin-2-yl)methyl)isoindoline-1,3-dione was synthesized using a similar method to 2-((8-bromo-6-cyclopropylimidazo[1,2-a]pyridin-2-yl)methyl)isoindoline-1,3-dione, starting from 5-bromo-3-chloropyridin-2-amine. ESI-MS (M+H)+: 352.1, ¹H NMR (400 MHz, CDCl₃) δ 7.91-7.85 (m, 2H), 7.76-7.70 (m, 3H), 7.46 (s, 1H), 6.99 (s, 1H), 5.10 (s, 2H), 1.87-1.80 (m, 1H), 0.99-0.89 (m, 2H), 0.67-0.60 (m, 2H).

### Synthesis of (8-chloro-6-cyclopropylimidazo[1,2-a]pyridin-2-yl)methanamine

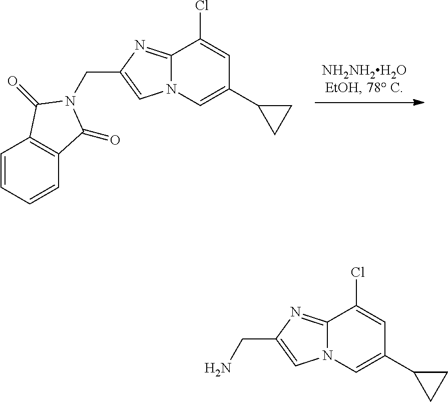 [SMILES: O=C1c2ccccc2C(=O)N1Cc1cn2cc(C3CC3)cc(Cl)c2n1 ; "NH2NH2·H2OEtOH, 78° C." ; NCc1cn2cc(C3CC3)cc(Cl)c2n1]

Using a similar procedure to that for 3-(2-(aminomethyl)-6-cyclopropylimidazo[1,2-a]pyridin-8-yl)oxetan-3-ol, (8-chloro-6-cyclopropylimidazo[1,2-a]pyridin-2-yl)methanamine (75 mg, 78%) was synthesised from 2-((8-chloro-6-cyclopropylimidazo[1,2-a]pyridin-2-yl)methyl)isoindoline-1,3-dione (152 mg, 0.432 mmol) using hydrazine hydrate. ESI-MS (M+H)+: 222.2, ¹H NMR (400 MHz, MeOD) δ 8.18-8.15 (m, 1H), 7.76-7.73 (m, 1H), 7.20-7.17 (m, 1H), 3.97 (s, 2H), 2.00-1.89 (m, 1H), 1.04-0.96 (m, 2H), 0.77-0.69 (m, 2H), exchangeable protons not observed.

### Synthesis of 1-(2-(aminomethyl)-6-cyclopropylimidazo[1,2-a]pyridin-8-yl)pyrrolidin-2-one

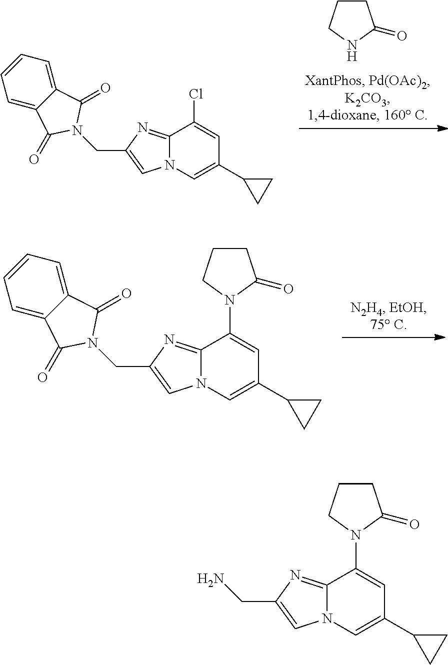

### Synthesis of 2-((6-cyclopropyl-8-(2-oxopyrrolidin-1-yl)imidazo[1,2-a]pyridin-2-yl)methyl)isoindoline-1,3-dione

Pyrrolidine-2-one (0.65 mL, 8.5 mmol), 2-((8-chloro-6-cyclopropylimidazo[1,2-a]pyridin-2-yl)methyl)isoindoline-1,3-dione (2000 mg, 5.7 mmol), Xantphos (660 mg, 1.1 mmol) and K₂CO₃ (1600 mg, 11 mmol) in 1,4-dioxane (20 mL) was degassed for 5 min with N₂. Pd(OAc)₂ (130 mg, 0.57 mmol) was added and the reaction mixture was heated in a microwave at 160° C. for 2 h. The mixture was cooled to room temperature, filtered through Celite® and concentrated in vacuo. The residue was purified by silica gel chromatography eluting with a gradient of 20-100% EtOAc in cyclohexane to give the title compound (1.0 g, 44%). ESI-MS (M+H)+: 401.3, ¹H NMR (400 MHz, CDCl₃) δ 7.87 (dd, J=3.0, 5.5 Hz, 2H), 7.72 (dd, J=3.1, 5.5 Hz, 2H), 7.69-7.68 (m, 1H), 7.45 (s, 1H), 7.24 (d, J=1.5 Hz, 1H), 5.02 (s, 2H), 4.27 (dd, J=7.1, 7.1 Hz, 2H), 2.58 (t, J=8.2 Hz, 2H), 2.22-2.13 (m, 2H), 1.90-1.82 (m, 1H), 0.95-0.89 (m, 2H), 0.68-0.62 (m, 2H).

### Synthesis of 1-(2-(aminomethyl)-6-cyclopropylimidazo[1,2-a]pyridin-8-yl)pyrrolidin-2-one

Using a similar procedure to that for 3-(2-(aminomethyl)-6-cyclopropylimidazo[1,2-a]pyridin-8-yl)oxetan-3-ol, 1-(2-(aminomethyl)-6-cyclopropylimidazo[1,2-a]pyridin-8-yl)pyrrolidin-2-one (600 mg, 89%) was synthesised from 2-((6-cyclopropyl-8-(2-oxopyrrolidin-1-yl)imidazo[1,2-a]pyridin-2-yl)methyl)isoindoline-1,3-dione (1.0 g, 2.50 mmol) using hydrazine hydrate. ESI-MS (M+H)+: 271.3, ¹H NMR (400 MHz, CDCl₃) δ 7.77 (s, 1H), 7.40 (s, 1H), 7.19 (d, J=1.5 Hz, 1H), 4.28 (t, J=7.2 Hz, 2H), 3.99 (d, J=0.6 Hz, 2H), 2.62 (t, J=8.2 Hz, 2H), 2.28-2.20 (m, 2H), 1.93-1.85 (m, 1H), 0.97-0.91 (m, 2H), 0.72-0.66 (m, 2H). NH₂ not observed.

The compounds in Table A6 were synthesized using a similar method to 1-(2-(aminomethyl)-6-cyclopropylimidazo[1,2-a]pyridin-8-yl)pyrrolidin-2-one from 2-((8-chloro-6-cyclopropylimidazo[1,2-a]pyridin-2-yl)methyl)isoindoline-1,3-dione and the appropriate coupling partner, followed by treatment with hydrazine hydrate:

TABLE A6

| Compound | Starting Material | Analytical Data |
|---|---|---|
| 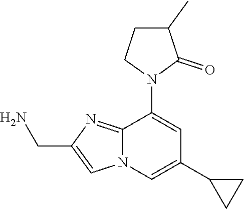 [SMILES: CC1CCN(c2cc(C3CC3)cn3cc(CN)nc23)C1=O] | 3-methylpyrrolidin-2-one | ESI-MS (M + H)+: 285.2, ¹H NMR (400 MHz, MeOD) δ 8.24- 8.23 (m, 1H), 7.80 (s, 1H), 7.15 (d, J = 1.6 Hz, 1H), 4.06 (s, 2H), 4.02-3.97 (m, 2H), 2.87-2.76 (m, 1H), 2.55-2.46 (m, 1H), 2.04-1.96 (m, 2H), 1.34 (d, J = 7.2 Hz, 3H), 1.05-0.99 (m, 2H), 0.80-0.75 (m, 2H). NH₂ not observed |
| 1-(2-(aminomethyl)-6- | | |
| cyclopropylimidazo[1,2-a]pyridin- | | |
| 8-yl)-3-methylpyrrolidin-2-one | | |
| 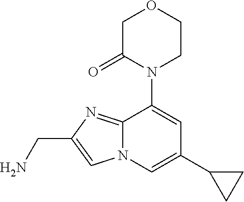 [SMILES: NCc1cn2cc(C3CC3)cc(N3CCOCC3=O)c2n1] | morpholin-3-one | ESI-MS [M + H]+: 287.2, ¹H NMR (400 MHz, MeOD) δ 8.25- 8.24 (m, 1H), 7.74-7.73 (m, 1H), 7.13 (d, J = 1.6 Hz, 1H), 4.36 (s, 2H), 4.15-4.12 (m, 2H), 3.93- 3.93 (m, 2H), 3.84-3.80 (m, 2H), 2.02-1.94 (m, 1H), 1.02- 0.97 (m, 2H), 0.78-0.73 (m, 2H), exchangeable NH₂ signal not observed. |
| 4-(2-(aminomethyl)-6- | | |
| cyclopropylimidazo[1,2-a]pyridin- | | |
| 8-yl)morpholin-3-one | | |
| 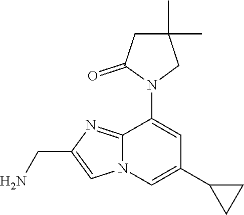 [SMILES: CC1(C)CC(=O)N(c2cc(C3CC3)cn3cc(CN)nc23)C1] | 4,4-dimethyl-2- pyrrolidinone | ESI-MS [M + H]+: 299, ¹H NMR (400 MHz, MeOD) δ 8.18 (s, 1H), 7.73 (s, 1H), 7.11 (d, J = 1.5 Hz, 1H), 3.97 (s, 2H), 3.78 (s, 2H), 2.48 (s, 2H), 2.01-1.93 (m, 1H), 1.32 (s, 6H), 1.02-0.95 (m, 2H), 0.77-0.71 (m, 2H). |
| 1-(2-(aminomethyl)-6- | | |
| cyclopropylimidazo[1,2-a]pyridin- | | |
| 8-yl)-4,4-dimethylpyrrolidin-2-one | | |
| 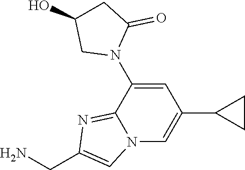 [SMILES: NCc1cn2cc(C3CC3)cc(N3C[C@@H](O)CC3=O)c2n1] | (S)-2-((6-cyclopropyl-8-(4- hydroxy-2-oxopyrrolidin-1- yl)imidazo[1,2-a]pyridin-2- yl)methyl)isoindoline-1,3- dione | ESI-MS (M + H)+: 287.2, ¹H NMR (400 MHz, MeOD) δ 8.24- 8.24 (m, 1H), 7.76-7.75 (m, 1H), 7.16 (d, J = 1.5 Hz, 1H), 4.64- 4.64 (m, 1H), 4.37 (dd, J = 5.4, 10.7 Hz, 1H), 3.96 (s, 2H), 3.80- 3.76 (m, 1H), 3.02 (dd, J = 6.5, 17.4 Hz, 1H), 2.55-2.49 (m, 1H), 2.04-1.96 (m, 1H), 1.05- 0.99 (m, 2H), 0.80-0.75 (m, |
| (S)-1-(2-(aminomethyl)-6- | | 2H), exchangeable protons not |
| cyclopropylimidazo[1,2-a]pyridin- | | observed. |
| 8-yl)-4-hydroxypyrrolidin-2-one | | |
| 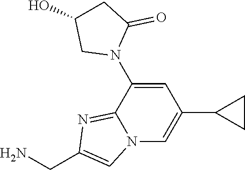 [SMILES: NCc1cn2cc(C3CC3)cc(N3C[C@H](O)CC3=O)c2n1] | (R)-2-((6-cyclopropyl-8-(4- hydroxy-2-oxopyrrolidin-1- yl)imidazo[1,2-a]pyridin-2- yl)methyl)isoindoline-1,3- dione | ESI-MS (M + H)+: 287, ¹H NMR (400 MHz, MeOD) δ 8.24-8.24 (m, 1H), 7.76-7.75 (m, 1H), 7.17-7.16 (m, 1H), 4.64-4.64 (m, 1H), 4.37 (dd, J = 5.4, 10.7 Hz, 1H), 3.97-3.96 (m, 2H), 3.80-3.76 (m, 1H), 3.02 (dd, J = 6.5, 17.3 Hz, 1H), 2.55-2.49 (m, 1H), 2.04-1.96 (m, 1H), 1.05-0.99 (m, 2H), 0.80-0.75 |
| (R)-1-(2-(aminomethyl)-6- | | (m, 2H), exchangeable protons |
| cyclopropylimidazo[1,2-a]pyridin- | | not observed. |
| 8-yl)-4-hydroxypyrrolidin-2-one | | |
| 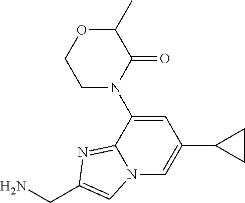 [SMILES: CC1OCCN(c2cc(C3CC3)cn3cc(CN)nc23)C1=O] | 2-((6-cyclopropyl-8-(2- methyl-3- oxomorpholino)imidazo[1,2- a]pyridin-2- yl)methyl)isoindoline-1,3- dione | ESI-MS (M + H)+: 301.2, ¹H NMR (400 MHz, DMSO) δ 8.34- 8.33 (m, 1H), 7.73 (s, 1H), 6.97- 6.96 (m, 1H), 4.39-4.35 (m, 1H), 4.09 (s, 1H), 4.00-3.96 (m, 2H), 3.84-3.80 (m, 2H), 3.75- 3.70 (m, 1H), 1.98-1.92 (m, 1H), 1.41 (d, J-6.8 Hz, 3H), 0.97- 0.92 (m, 2H), 0.72-0.67 (m, 2H), exchangeable protons not observed. |
| 4-(2-(aminomethyl)-6- | | |
| cyclopropylimidazo[1,2-a]pyridin- | | |
| 8-yl)-2-methylmorpholin-3-one | | |

### Synthesis of 1-(2-(aminomethyl)-6-cyclopropylimidazo[1,2-a]pyridin-8-yl)imidazolidine-2,4-dione

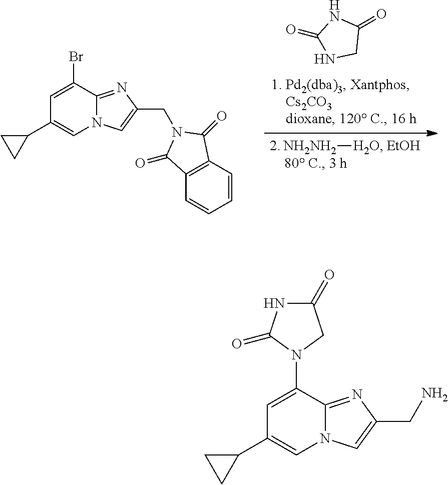

### Synthesis of 2-((6-cyclopropyl-8-(2,4-dioxoimidazolidin-1-yl)imidazo[1,2-a]pyridin-2-yl)methyl)isoindoline-1,3-dione

A mixture of 2-((8-bromo-6-cyclopropylimidazo[1,2-a]pyridin-2-yl)methyl)isoindoline-1,3-dione (350 mg, 0.886 mmol), imidazolidine-2,4-dione (354 mg, 3.544 mmol), Pd₂(dba)₃ (162 mg, 0.177 mmol), Xantphos (154 mg, 0.266 mmol) and Cs₂CO₃ (866 mg, 2.658 mmol) in 1,4-dioxane (20 ml) were stirred at 120° C. for 16 h. The reaction mixture was allowed to reach room temperature, quenched with water (50 mL) and extracted with EtOAc (3×50 ml). The combined organics were washed with brine (50 mL), dried over Na₂SO₄ and concentrated in vacuo. The crude residue was purified by preparative TLC (eluent: DCM:MeOH=15:1) to afford 2-((6-cyclopropyl-8-(2,4-dioxoimidazolidin-1-yl)imidazo[1,2-a]pyridin-2-yl)methyl)isoindoline-1,3-dione (70 mg, 19%) as a yellow solid.

ESI-MS: [M+H]⁺, 416.1

### Synthesis of 1-(2-(aminomethyl)-6-cyclopropylimidazo[1,2-a]pyridin-8-yl)imidazolidine-2,4-dione

A solution of 2-((6-cyclopropyl-8-(2,4-dioxoimidazolidin-1-yl)imidazo[1,2-a]pyridin-2-yl)methyl) isoindoline-1,3-dione (70 mg, 0.17 mmol) and NH₂NH₂—H₂O (0.2 mL) in EtOH (1 ml) was stirred at 80° C. for 3 h. The reaction mixture was allowed to reach room temperature, solid was filtered and filtrate was concentrated to give 1-(2-(aminomethyl)-6-cyclopropylimidazo[1,2-a]pyridin-8-yl)imidazolidine-2,4-dione (40 mg, crude) which was used in next step without further purification. ESI-MS: [M+H]⁺, 286.2

Using a similar procedure to that used for 1-(2-(aminomethyl)-6-cyclopropylimidazo[1,2-a]pyridin-8-yl)imidazolidine-2,4-dione, the compounds in Table A7 were prepared from 2-((8-bromo-6-cyclopropylimidazo[1,2-a]pyridin-2-yl)methyl)isoindoline-1,3-dione and an appropriate coupling partner, followed by treatment with hydrazine hydrate.

TABLE A7

| Structure | Coupling Partner | Analytical Data |
|---|---|---|
| 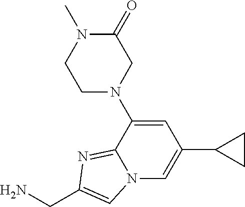 [SMILES: CN1CCN(c2cc(C3CC3)cn3cc(CN)nc23)CC1=O] | 1-methylpiperazin- 2-one | ¹H NMR (400 MHz, DMSO) δ 7.97 (s, 1H), 7.63 (s, 1H), 6.20 (d, J = 1.5 Hz, 1H), 4.12 (s, 2H), 3.92 (m, 2H), 3.83 (s, 2H), 3.50 (m, 2H), 3.22 (s, 2H), 2.96 (s, 3H), 1.97-1.88 (m, 1H), 0.93 (m, 2H), 0.77- 0.72 (m, 2H). |
| 4-(2-(aminomethyl)-6- | | |
| cyclopropylimidazo[1,2- | | |
| a]pyridin-8-yl)-1- | | |
| methylpiperazin-2-one | | |
| 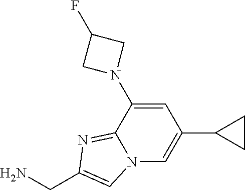 [SMILES: NCc1cn2cc(C3CC3)cc(N3CC(F)C3)c2n1] | 3-fluoroazetidine | ESI-MS (M + H)+: 261.2. |
| (6-cyclopropyl-8-(3- | | |
| fluoroazetidin-1-yl)imidazo[1,2- | | |
| a]pyridin-2-yl)methanamine | | |
| 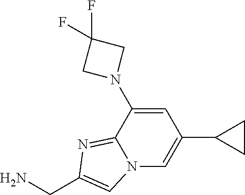 [SMILES: NCc1cn2cc(C3CC3)cc(N3CC(F)(F)C3)c2n1] | 3,3- difluoroazetidine | ESI-MS (M + H)+: 279.2. |
| (6-cyclopropyl-8-(3,3- | | |
| difluoroazetidin-1- | | |
| yl)imidazo[1,2-a]pyridin-2- | | |
| yl)methanamine | | |
| 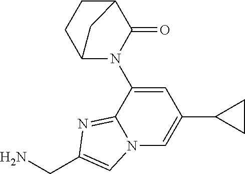 [SMILES: NCc1cn2cc(C3CC3)cc(N3C(=O)C4CCC3C4)c2n1] | 2- azabicyclo[2.2.1] heptan-3-one | ¹H NMR (400 MHz, DMSO) δ 8.17 (s, 1H), 7.69 (s, 1H), 7.29 (s, 1H), 5.47 (s, 1H), 3.81 (s, 2H), 2.83 (m, 1H), 2.02 (m, 1H), 1.97-1.73 (m, 4H), 1.65-1.53 (m, 2H), 0.94-0.90 (m, 2H), 0.67-0.61 (m, 2H). |
| 2-(2-(aminomethyl)-6- | | |
| cyclopropylimidazo[1,2- | | |
| a]pyridin-8-yl)-2- | | |
| azabicyclo[2.2.1]heptan-3-one | | |
| 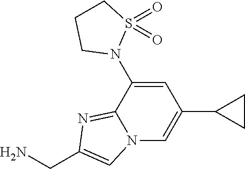 [SMILES: NCc1cn2cc(C3CC3)cc(N3CCCS3(=O)=O)c2n1] | isothiazolidine 1,1- dioxide | ESI-MS (M + H)+: 307.2 |
| 2-(2-(aminomethyl)-6- | | |
| cyclopropylimidazo[1,2- | | |
| a]pyridin-8-yl)isothiazolidine | | |
| 1,1-dioxide | | |
| 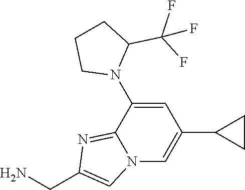 [SMILES: NCc1cn2cc(C3CC3)cc(N3CCCC3C(F)(F)F)c2n1] | 2- (trifluoromethyl) pyrrolidine | ¹H NMR (400 MHz, DMSO) δ 7.83 (s, 1H), 7.61 (s, 1H), 6.42-6.33 (m, 1H), 6.10 (s, 1H), 3.94-3.85 (m, 1H), 3.81 (s, 2H), 3.47 (s, 1H), 2.34-2.21 (m, 1H), 2.17- 2.05 (m, 3H), 1.95-1.86 (m, 1H), 0.92 (m, 2H), 0.77-0.67 (m, 2H). |
| (6-cyclopropyl-8-(2- | | |
| (trifluoromethyl)pyrrolidin-1- | | |
| yl)imidazo[1,2-a]pyridin-2- | | |
| yl)methanamine | | |
| 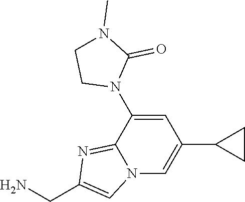 [SMILES: CN1CCN(c2cc(C3CC3)cn3cc(CN)nc23)C1=O] | 1- methylimidazolidin- 2-one | ¹H NMR (400 MHz, CDCl₃) δ 7.79 (s, 1H), 7.40 (s, 1H), 7.03 (d, J = 1.6 Hz, 1H), 5.04 (dd, J-6.3, 13.6 Hz, 1H), 3.99 (d, J = 1.8 Hz, 2H), 2.67-2.61 (m, 2H), 2.59- 2.45 (m, 1H), 1.93-1.86 (m, 1H), 1.82- 1.74 (m, 1H), 1.07 (d, J = 6.3 Hz, 3H), 0.97- 0.93 (m, 2H), 0.72-0.67 (m, 2H). |
| 1-(2-(aminomethyl)-6- | | |
| cyclopropylimidazo[1,2- | | |
| a]pyridin-8-yl)-3- | | |
| methylimidazolidin-2-one | | |
| 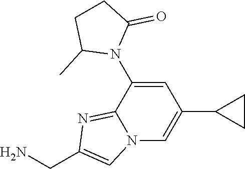 | 5- methylpyrrolidin- 2-one | ESI-MS (M + H)+: 415.4, ¹H NMR (400 MHz, CDCl₃) δ 7.89-7.86 (m, 2H), 7.74-7.70 (m, 3H), 7.44 (s, 1H), 7.07-7.06 (m, 1H), 5.13-5.04 (m, 1H), 5.03-5.02 (m, 2H), 2.63-2.58 (m, 2H), 2.48-2.39 (m, 1H), 1.90-1.82 (m, 1H), 1.75-1.65 (m, 1H), 1.00 (d, J = 6.3 Hz, 3H), 0.95-0.90 (m, 2H), 0.68-0.63 (m, 2H). |
| 1-(2-(aminomethyl)-6- | | |
| cyclopropylimidazo[1,2- | | |
| a]pyridin-8-yl)-5- | | |
| methylpyrrolidin-2-one | | |
| 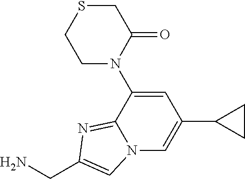 [SMILES: NCc1cn2cc(C3CC3)cc(N3CCSCC3=O)c2n1] | thiomorpholin-3- one | ¹H NMR (400 MHz, DMSO) δ 8.31 (d, J = 1.5 Hz, 1H), 7.73 (s, 1H), 6.93 (d, J = 1.5 Hz, 1H), 4.01 (t, J = 5.8, 2H), 3.80 (s, 2H), 3.47 (s, 2H), 3.18 (m, 2H), 1.99-1.91 (m, 1H), 0.97-0.91 (m, 2H), 0.71-0.66 (m, 2H). |
| 4-(2-(aminomethyl)-6- | | |
| cyclopropylimidazo[1,2- | | |
| a]pyridin-8-yl)thiomorpholin-3- | | |
| one | | |
| 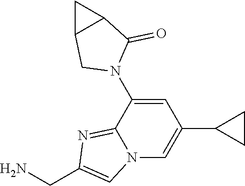 [SMILES: NCc1cn2cc(C3CC3)cc(N3CC4CC4C3=O)c2n1] | 3- azabicyclo[3.1.0] hexan-2-one | ESI-MS (M + H)+: 283.1, ¹H NMR (400 MHz, CDCl₃) δ 7.73 (s, 1H), 7.38 (s, 1H), 7.09 (s, 1H), 4.52 (m, 1H), 4.12 (m, 1H), 3.99 (s, 2H), 2.12-2.02 (m, 2H), 1.93- 1.83 (m, 1H), 1.28-1.21 (m, 2H), 0.99- 0.87 (m, 2H), 0.69-0.64 (m, 2H). |
| 3-(2-(aminomethyl)-6- | | |
| cyclopropylimidazo[1,2- | | |
| a]pyridin-8-yl)-3- | | |
| azabicyclo[3.1.0]hexan-2-one | | |
| 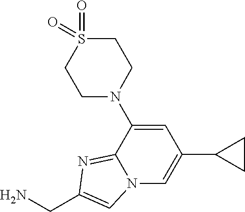 [SMILES: NCc1cn2cc(C3CC3)cc(N3CCS(=O)(=O)CC3)c2n1] | thiomorpholine 1,1-dioxide | ¹H NMR (400 MHz, DMSO) δ 7.94 (s, 1H), 7.60 (s, 1H), 6.28 (s, 1H), 4.12-4.11 (m, 4H), 3.78 (s, 2H), 3.24 (m, 4H), 1.93- 1.85 (m, 1H), 0.92-0.86 (m, 2H), 0.75- 0.70 (m, 2H). |
| 4-(2-(aminomethyl)-6- | | |
| cyclopropylimidazo[1,2- | | |
| a]pyridin-8-yl)thiomorpholine | | |
| 1,1-dioxide | | |
| 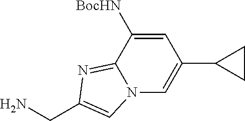 [SMILES: CC(C)(C)OC(=O)Nc1cc(C2CC2)cn2cc(CN)nc12] | tert-Butyl carbamate | ESI-MS (M + H)+: 303.1 |
| tert-butyl (2-(aminomethyl)-6- | | |
| cyclopropylimidazo[1,2- | | |
| a]pyridin-8-yl)carbamate | | |

### Synthesis of 2-(methoxymethyl)-1-methylpiperazine

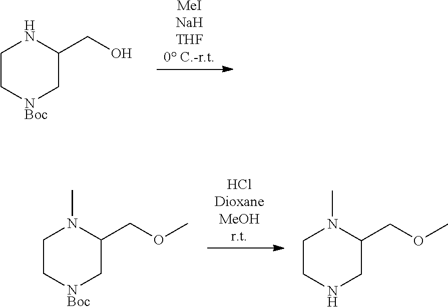

### Synthesis of tert-butyl 3-(methoxymethyl)-4-methylpiperazine-1-carboxylate

NaH (60% in mineral oil, 0.041 g, 1.0 mmol) was added to a stirred solution of tert-butyl 3-(hydroxymethyl)piperazine-1-carboxylate (0.20 g, 0.93 mmol) in THF (5.0 mL) at 0° C. The mixture was stirred at 0° C. for 30 min then MeI (0.063 mL, 1.0 mmol) was added. The mixture was stirred at 0° C. for 2 h then quenched with water and extracted with DCM (3×). The combined organic layers were dried over MgSO₄, filtered and concentrated in vacuo. The residue was purified by column chromatography on silica gel, eluting with 0-20% (7 N NH₃ in MeOH) in DCM to give the title compound (0.060 g, 28%). ¹H NMR (400 MHz, CDCl₃) δ 3.97-3.77 (m, 2H), 3.52-3.37 (m, 2H), 3.35 (s, 3H), 3.07-2.92 (m, 2H), 2.77-2.70 (m, 1H), 2.34 (s, 3H), 2.24-2.14 (m, 1H), 2.14-2.06 (m, 1H), 1.46 (s, 9H).

### Synthesis of 2-(methoxymethyl)-1-methylpiperazine

A mixture of tert-butyl 3-(methoxymethyl)-4-methylpiperazine-1-carboxylate (0.060 g, 0.25 mmol) and HCl (4.0 M in 1,4-dioxane, 0.61 mL, 2.5 mmol) in MeOH (1.0 mL) was stirred at room temperature for 2 h. The mixture was concentrated in vacuo to give the title compound (0.035 g, quant.) as an off-white foam which was used without further purification.

### Synthesis of 3-methylimidazolidin-4-one

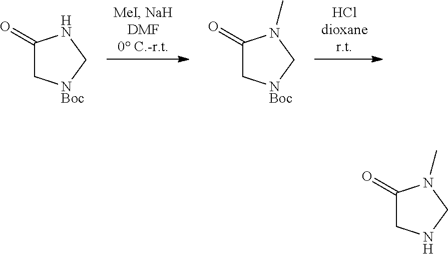

### Synthesis of tert-butyl 3-methyl-4-oxoimidazolidine-1-carboxylate

NaH (60% in mineral oil, 0.20 g, 4.9 mmol) was added to a stirred solution of tert-butyl 4-oxoimidazolidine-1-carboxylate (0.76 g, 4.1 mmol) in DMF (5.0 mL) at 0° C. After 20 min MeI (0.36 mL, 5.7 mmol) was added and the mixture was warmed to room temperature and stirred for 2 h. NaHCO₃ (sat·aq·) was added and the mixture was extracted with EtOAc (3×). The combined organic layers were washed with brine (sat·aq·), dried over MgSO₄, filtered and concentrated in vacuo. The residue was purified by column chromatography on silica gel, eluting with 0-100% EtOAc in DCM to give the title compound (0.54 g, 66%) as an off-white solid. ESI-MS (M+H)+: 201.2, ¹H NMR (400 MHz, CDCl₃) δ 4.71 (d, J=8.3 Hz, 2H), 3.97-3.87 (m, 2H), 2.96 (s, 3H), 1.48 (s, 9H).

### Synthesis of 3-methylimidazolidin-4-one

A mixture of tert-butyl 3-methyl-4-oxoimidazolidine-1-carboxylate (0.54 g, 2.7 mmol) and HCl solution (4.0 M in 1,4-dioxane, 6.7 mL, 27 mmol) in MeOH (5.0 mL) was stirred at room temperature for 2 h. The mixture was concentrated in vacuo and the residue was loaded onto an SCX cartridge and washed with a mixture of MeOH and DCM. The product was eluted with (7 N NH₃ in MeOH) in DCM to give the title compound (0.23 g, 85%) as a clear oil. ESI-MS (M+H)+: 101.3, ¹H NMR (400 MHz, CDCl₃) δ 4.39 (s, 2H), 3.47 (d, J=7.3 Hz, 2H), 2.86 (s, 3H).

### Synthesis of 3-methylimidazolidine-2,4-dione

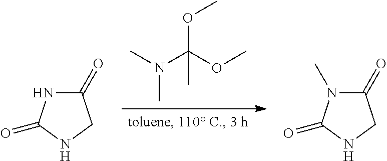

To a solution of imidazolidine-2,4-dione (1 g, 10 mmol) in dry toluene (25 mL) was added 1,1-dimethoxy-N,N-dimethylethan-1-amine (4 g, 30 mmol). The reaction mixture was stirred at 110° C. for 3 h under N₂ and then cooled to 0° C. for 15 minutes and filtered. The filtrate was concentrated and the crude residue was purified by silica gel chromatography (eluent: DCM/MeOH=10/1) to give 3-methylimidazolidine-2,4-dione (400 mg, 34%) as a white solid. ESI-MS [M+H]⁺: 115.1.

### Synthesis of 4-(2-(aminomethyl)-6-cyclopropylimidazo[1,2-a]pyridin-8-yl)piperazin-2-one

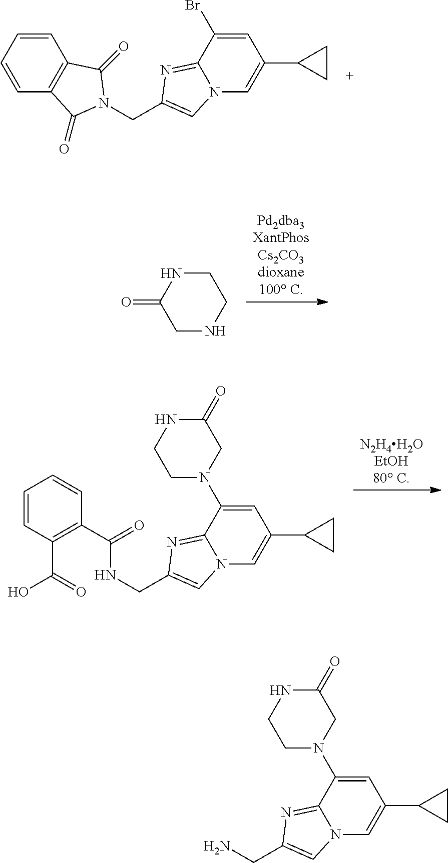 [SMILES: O=C1c2ccccc2C(=O)N1Cc1cn2cc(C3CC3)cc(Br)c2n1 ; "Pd2dba3XantPhosCs2CO3dioxane100° C." ; O=C1CNCCN1 ; O=C1CN(c2cc(C3CC3)cn3cc(CNC(=O)c4ccccc4C(=O)O)nc23)CCN1 ; "N2H4·H2OEtOH80° C." ; NCc1cn2cc(C3CC3)cc(N3CCNC(=O)C3)c2n1]

### Synthesis of 2-(((6-cyclopropyl-8-(3-oxopiperazin-1-yl)imidazo[1,2-a]pyridin-2-yl)methyl)carbamoyl)benzoic acid

A mixture of 2-((8-bromo-6-cyclopropylimidazo[1,2-a]pyridin-2-yl)methyl)isoindoline-1,3-dione (0.50 g, 1.3 mmol), piperazin-2-one (0.19 g, 1.9 mmol), Pd₂(dba)₃ (0.12 g, 0.13 mmol), XantPhos (0.15 g, 0.25 mmol) and Cs₂CO₃ (0.82 g, 2.5 mmol) in 1,4-dioxane (13 mL) was degassed with nitrogen then stirred at 100° C. for 2.5 h. The mixture was filtered through Celite® with a mixture of 7 N NH₃ in MeOH and DCM and concentrated in vacuo. The residue was purified by column chromatography on silica gel (40 g), eluting with 1-100% (7 N NH₃ in MeOH) in DCM to give the title compound (0.68 g, quant.), which was used directly in the next step. ESI-MS (M+H)+: 434.4.

### Synthesis of 4-(2-(aminomethyl)-6-cyclopropylimidazo[1,2-a]pyridin-8-yl)piperazin-2-one

A mixture of 2-(((6-cyclopropyl-8-(3-oxopiperazin-1-yl)imidazo[1,2-a]pyridin-2-yl)methyl)carbamoyl)benzoic acid (0.68 g, 1.6 mmol) and hydrazine hydrate (0.98 mL, 16 mmol) in ethanol (16 mL) was stirred at 80° C. for 18 h. The mixture was loaded onto an SCX cartridge and washed with a mixture of MeOH and DCM. The product was eluted with 7 N NH₃ in MeOH in DCM to give the title compound (0.18 g, 39%) as a yellow gum, which was used without further purification. ESI-MS (M+H)+: 286.3.

### Synthesis of 2-((6-cyclopropyl-8-(2-oxo-3-azabicyclo[3.1.0]hexan-3-yl)imidazo[1,2-a]pyridin-2-yl)methyl)isoindoline-1,3-dione

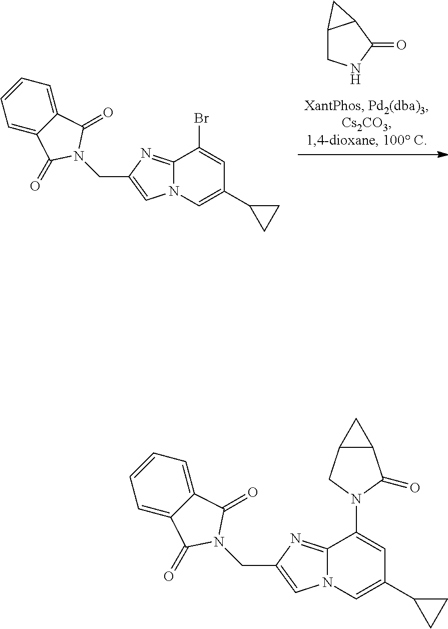 [SMILES: O=C1NCC2CC12 ; O=C1c2ccccc2C(=O)N1Cc1cn2cc(C3CC3)cc(Br)c2n1 ; "XantPhos, Pd2(dba)3,Cs2CO3,1,4-dioxane, 100° C." ; O=C1c2ccccc2C(=O)N1Cc1cn2cc(C3CC3)cc(N3CC4CC4C3=O)c2n1]

3-azabicyclo[3.1.0]hexan-2-one (270 mg, 2.8 mmol), 2-((8-bromo-6-cyclopropylimidazo[1,2-a]pyridin-2-yl)methyl)isoindoline-1,3-dione (730 mg, 1.9 mmol), XantPhos (210 mg, 0.37 mmol), and Cs₂CO₃ (1.2 g, 3.7 mmol) were dissolved in 1,4-dioxane (10 mL) and degassed with nitrogen for 5 min. Pd₂(dba)₃ (170 mg, 0.185 mmol) was added and the reaction mixture was heated to 100° C. and stirred for 4 h. The reaction mixture was cooled to room temperature, filtered through Celite® and concentrated in vacuo. The residue was purified by silica gel chromatography eluting with a gradient of 20-100% EtOAc in cyclohexane to give the title compound (580 mg, 76%) which was used without further purification. ESI-MS (M+H)+: 413.2 ¹H NMR (400 MHz, CDCl₃): δ 7.88 (dd, J=3.0, 5.4 Hz, 2H), 7.73 (dd, J=3.0, 5.4 Hz, 2H), 7.66 (dd, J=0.7, 1.5 Hz, 1H), 7.43 (s, 1H), 7.14 (d, J=1.5 Hz, 1H), 5.01 (s, 2H), 4.45 (dd, J=5.9, 10.5 Hz, 1H), 4.18-4.14 (m, 1H), 2.08-2.03 (m, 1H), 2.01-1.95 (m, 1H), 1.87-1.80 (m, 1H), 1.20-1.14 (m, 1H), 0.93-0.86 (m, 3H), 0.66-0.61 (m, 2H).

### Synthesis of 1-(2-(aminomethyl)-6-cyclopropylimidazo[1,2-a]pyridin-8-yl)-3-tritylimidazolidin-2-one

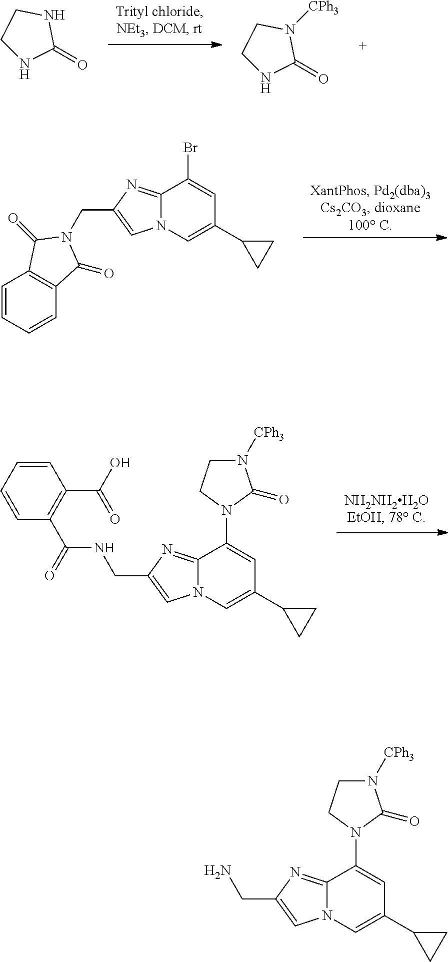

### Synthesis of 1-tritylimidazolidin-2-one

Triethylamine (1.6 mL, 11.62 mmol) was added to a stirred solution of imidazolidin-2-one (500 mg, 5.81 mmol) and trityl chloride (2.43 g, 8.71 mmol) in DCM (15 mL). The reaction was stirred at room temperature for 18 h. The reaction was diluted with water (50 mL) and extracted with DCM (3×30 mL). The organics were combined, dried over MgSO₄ then concentrated in vacuo. The residue was purified by silica gel chromatography, eluting with a gradient of 0-10% MeOH in DCM to give the title compound (1.44 g, 75%). ESI-MS (M+H)+: 327.2, ¹H NMR (400 MHz, DMSO) δ 7.39 (d, J=7.5 Hz, 6H), 7.29 (dd, J=7.7, 7.7 Hz, 6H), 7.19 (dd, J=7.2, 7.2 Hz, 3H), 6.40 (s, 1H), 3.31-3.19 (m, 4H).

### Synthesis of 2-(((6-cyclopropyl-8-(2-oxo-3-tritylimidazolidin-1-yl)imidazo[1,2-a]pyridin-2-yl)methyl)carbamoyl)benzoic acid

2-((8-bromo-6-cyclopropylimidazo[1,2-a]pyridin-2-yl)methyl)isoindoline-1,3-dione (300 mg, 0.76 mmol), 1-tritylimidazolidin-2-one (370 mg, 1.14 mmol), Xantphos (88 mg, 0.15 mmol) and Cs₂CO₃ (490 mg, 1.51 mmol) were suspended in dioxane and degassed with N₂ for 5 min. Pd₂(dba)₃ (69 mg, 0.0757 mmol) was added and the reaction was heated to 100° C., under a N₂ environment, for 4 h. The reaction was cooled to room temperature and filtered through Celite®. The Celite® was washed with DCM:MeOH (20 mL 1:1), the filtrate was concentrated in vacuo. The residue was purified by silica gel chromatography, eluting with a gradient of 0-10% (7N NH₃ in MeOH) in DCM to give the title compound (320 mg, 64%). ESI-MS (M+H)+: 662.4, ¹H NMR (400 MHz, DMSO) δ 8.10 (s, 1H), 7.84 (s, 1H), 7.62 (d, J=7.0 Hz, 2H), 7.46 (d, J=7.6 Hz, 6H), 7.34 (dd, J=7.7, 7.7 Hz, 7H), 7.23 (dd, J=7.3, 7.3 Hz, 4H), 7.14 (d, J=1.5 Hz, 1H), 4.51 (d, J=5.5 Hz, 2H), 4.34 (dd, J=7.4, 7.4 Hz, 2H), 3.48 (dd, J=7.5, 7.5 Hz, 2H), 1.91-1.83 (m, 1H), 0.91-0.85 (m, 2H), 0.62-0.57 (m, 2H), Exchangeable protons not seen.

### Synthesis of 1-(2-(aminomethyl)-6-cyclopropylimidazo[1,2-a]pyridin-8-yl)-3-tritylimidazolidin-2-one

Hydrazine hydrate (0.13 mL, 2.42 mmol) was added to a stirred solution of 2-(((6-cyclopropyl-8-(2-oxo-3-tritylimidazolidin-1-yl)imidazo[1,2-a]pyridin-2-yl)methyl)carbamoyl)benzoic acid (320 mg, 0.484 mmol) in ethanol (5.0 mL) the reaction was then heated to 78° C. for 18 h. The reaction was cooled to room temperature and loaded onto a 5.0 g SCX cartridge in MeOH, washed with 3:1 DCM:MeOH and eluted with 3:1 DCM:7N NH₃ in MeOH. The eluent was concentrated in vacuo to give the title compound (140 mg, 57%), used in the next step without further purification. ESI-MS (M+H)+: 514.3, ¹H NMR (400 MHz, DMSO) δ 8.12 (d, J=1.4 Hz, 1H), 7.67 (s, 1H), 7.46 (d, J=7.5 Hz, 6H), 7.33 (dd, J=7.7, 7.7 Hz, 6H), 7.23 (dd, J=7.3, 7.3 Hz, 3H), 7.11 (d, J=1.5 Hz, 1H), 4.31 (dd, J=7.5, 7.5 Hz, 2H), 3.81 (s, 2H), 3.29-3.21 (m, 2H), 3.18 (s, 1H), 1.91-1.83 (m, 1H), 0.91-0.85 (m, 2H), 0.61-0.56 (m, 2H).

### Synthesis of tert-butyl 4-(2-(aminomethyl)-6-cyclopropylimidazo[1,2-a]pyridin-8-yl)-3-oxopiperazine-1-carboxylate

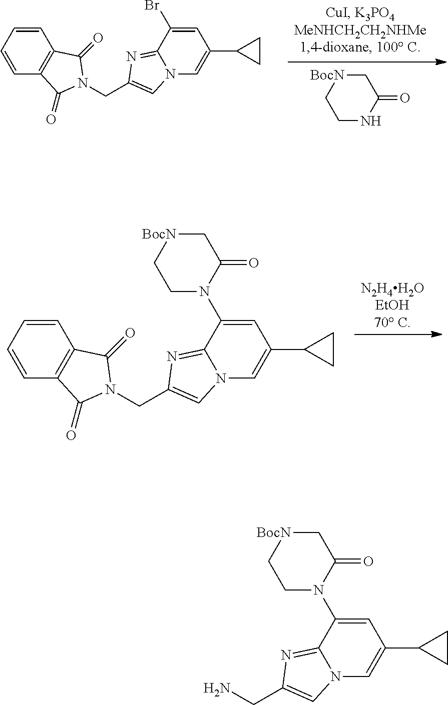

### Synthesis of tert-butyl 4-(6-cyclopropyl-2-((1,3-dioxoisoindolin-2-yl)methyl)imidazo[1,2-a]pyridin-8-yl)-3-oxopiperazine-1-carboxylate

A mixture of 2-((8-bromo-6-cyclopropylimidazo[1,2-a]pyridin-2-yl)methyl)isoindoline-1,3-dione (0.30 g, 0.76 mmol), tert-butyl 3-oxopiperazine-1-carboxylate (0.20 g, 0.98 mmol), CuI (0.029 g, 0.15 mmol) and N,N′-dimethylethylenediamine (0.033 mL, 0.30 mmol) in 1,4-dioxane (3.0 mL) was degassed with nitrogen and stirred at 115° C. for 18 h. K₃PO₄ (0.21 g, 0.98 mmol) and further CuI (0.029 g, 0.15 mmol) were added and the mixture was stirred at 115° C. for 18 h. The mixture was diluted with EtOAc and washed with NH₄OH (dil·aq·) and brine (sat·aq·). The organic layer was passed through a phase separator and concentrated in vacuo. The residue was purified by column chromatography on silica gel, eluting with 0-20% MeOH in DCM to give the title compound (0.12 g, 32%) as a beige solid. ESI-MS (M+H)+: 516, ¹H NMR (400 MHz, DMSO) δ 8.27 (d, J=10.7 Hz, 1H), 7.96-7.87 (m, 4H), 7.77 (d, J=9.3 Hz, 1H), 7.37 (d, J=9.3 Hz, 1H), 4.88 (s, 2H), 4.12 (s, 1H), 3.84-3.64 (m, 3H), 3.48-3.39 (m, 2H), 1.96-1.89 (m, 1H), 1.45 (d, J=18.2 Hz, 9H), 0.96-0.89 (m, 2H), 0.69-0.64 (m, 2H).

### Synthesis of tert-butyl 4-(2-(aminomethyl)-6-cyclopropylimidazo[1,2-a]pyridin-8-yl)-3-oxopiperazine-1-carboxylate

Hydrazine monohydrate (0.27 mL, 5.5 mmol) was added to a stirred solution of tert-butyl 4-(6-cyclopropyl-2-((1,3-dioxoisoindolin-2-yl)methyl)imidazo[1,2-a]pyridin-8-yl)-3-oxopiperazine-1-carboxylate (0.28 g, 0.55 mmol) in EtOH (1.7 mL). The mixture was stirred at 70° C. for 1 h. The mixture was diluted with EtOH and filtered with further EtOH. The filtrate was concentrated in vacuo. The residue was loaded onto an SCX cartridge and washed with MeOH. The product was eluted with 20% (7 N NH₃ in MeOH) in DCM and concentrated in vacuo to give the title compound (0.13 g, 59%) as a yellow gum. ESI-MS (M+H)+: 386.

### Synthesis of 1-(2-(aminomethyl)-6-cyclopropylimidazo[1,2-a]pyridin-8-yl)-4-methylpiperazin-2-one

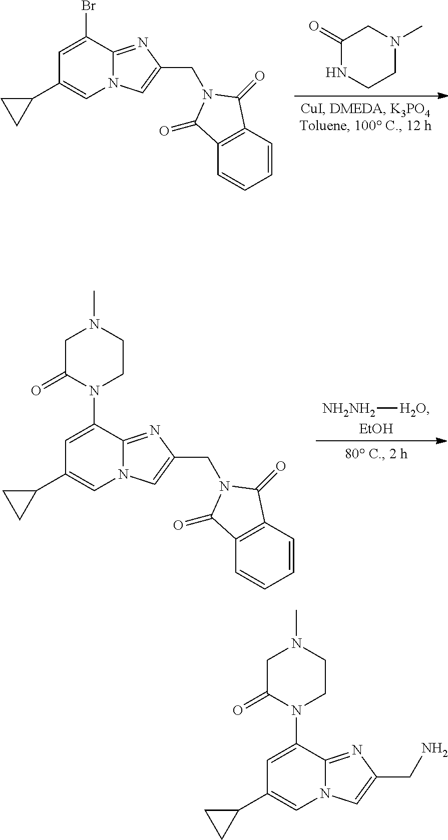

### Synthesis of 2-((6-cyclopropyl-8-(4-methyl-2-oxopiperazin-1-yl)imidazo[1,2-a]pyridin-2-yl)methyl)isoindoline-1,3-dione

To a mixture of 2-((8-bromo-6-cyclopropylimidazo[1,2-a]pyridin-2-yl)methyl)isoindoline-1,3-dione (500 mg, 1.26 mmol), 4-methylpiperazin-2-one (287.3 mg, 2.52 mmol) and K₃PO₄ (801.3 mg, 3.78 mmol) in toluene (10 mL) were added CuI (47.9 mg, 0.25 mmol) and DMEDA (221.7 mg, 2.52 mmol). The reaction mixture was stirred at 100° C. for 12 h under N₂ and cooled to room temperature. The reaction mixture was filtered through celite and the filter cake was washed with EtOAc (10 mL). The filtrate was concentrated in vacuo to give the crude product, which was purified by column chromatography (PE/EtOAc=4/1) to give 2-((6-cyclopropyl-8-(4-methyl-2-oxopiperazin-1-yl)imidazo[1,2-a]pyridin-2-yl)methyl)isoindoline-1,3-dione (120 mg, 22%) as a white solid. ESI-MS [M+H]+: 430.2.

### Synthesis of 1-(2-(aminomethyl)-6-cyclopropylimidazo[1,2-a]pyridin-8-yl)-4-methylpiperazin-2-one

To a solution of 2-((6-cyclopropyl-8-(4-methyl-2-oxopiperazin-1-yl)imidazo[1,2-a]pyridin-2-yl)methyl)isoindoline-1,3-dione (120 mg, 0.28 mmol) in EtOH (5 mL) was added hydrazine hydrate (77 mg, 1.54 mmol). The reaction mixture was stirred at 80° C. for 2 h and cooled to room temperature. The reaction mixture was filtered through celite and the filter cake was washed with DCM/MeOH (30/1, 100 mL). The filtrate was concentrated in vacuo to give the crude, which was purified by preparative TLC (DCM/MeOH=30/1) to give 1-(2-(aminomethyl)-6-cyclopropylimidazo[1,2-a]pyridin-8-yl)-4-methylpiperazin-2-one (60 mg, 72%) as a white solid. ESI-MS [M+H]+: 300.2.

### Synthesis of (6-cyclopropyl-8-(1-methylazetidin-3-yl)imidazo[1,2-a]pyridin-

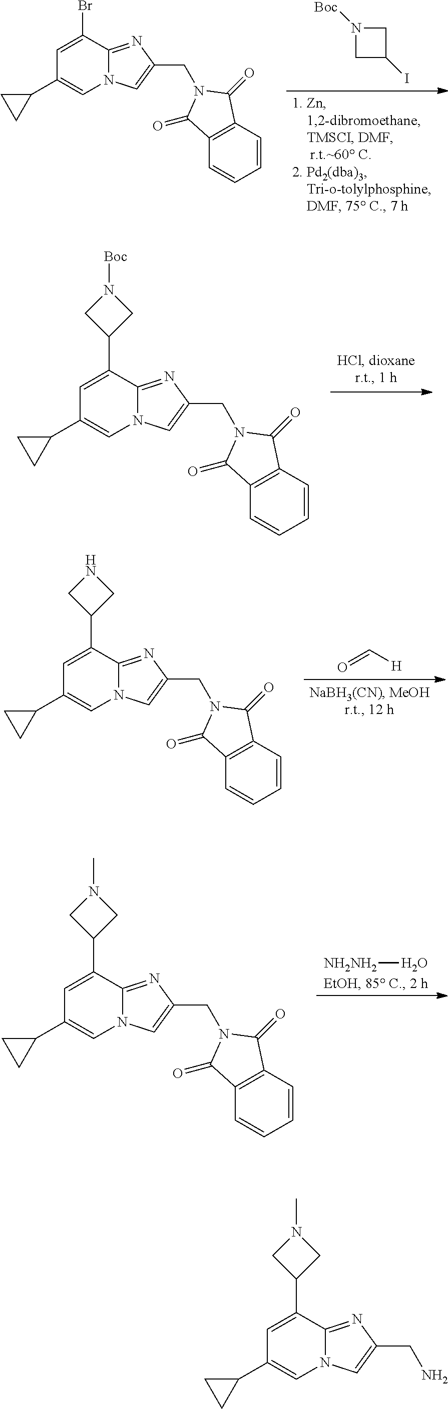

### Synthesis of tert-butyl 3-(6-cyclopropyl-2-((1,3-dioxoisoindolin-2-yl)methyl)imidazo[1,2-a]pyridin-8-yl)azetidine-1-carboxylate

1,2-Dibromoethane (95 mg, 0.51 mmol) was added to a suspension of zinc dust (3.28 g, 50.1 mmol) in dry DMF (50 mL), and the reaction mixture was heated to reflux for 1 h. After cooling to room temperature, the reaction mixture was treated with trimethylsilyl chloride (55 mg, 0.51 mmol) and stirred for 1 h. A solution of tert-butyl 3-iodoazetidine-1-carboxylate (2.86 g, 10.1 mmol) in DMF (15 mL) was then added drop-wise. The reaction was stirred for 1 h at 60° C. and cooled to room temperature. To this mixture were added 2-((8-bromo-6-cyclopropylimidazo[1,2-a]pyridin-2-yl)methyl)isoindoline-1,3-dione (2 g, 5.05 mmol) and Pd₂(dba)₃ (467 mg, 0.51 mmol) and tri-o-tolylphosphine (310 mg, 1.02 mmol) and the mixture was heated at 75° C. for 7 h. After cooling to room temperature the reaction mixture was filtered through Celite and the filtrate was concentrated in vacuo, then treated with EtOAc (3×50 mL) and saturated aqueous sodium carbonate solution. The combined organic layers were dried over Na₂SO₄, filtered and concentrated in vacuo to give the crude, which was purified with silica gel chromatography (eluent: DCM/MeOH=20/1) to give tert-butyl 3-(6-cyclopropyl-2-((1,3-dioxoisoindolin-2-yl)methyl)imidazo[1,2-a]pyridin-8-yl)azetidine-1-carboxylate (320 mg, 13%) as a yellow solid. ESI-MS [M+H]⁺: 473.2.

### Synthesis of 2-((8-(azetidin-3-yl)-6-cyclopropylimidazo[1,2-a]pyridin-2-yl)methyl)isoindoline-1,3-dione

To a solution of tert-butyl 3-(6-cyclopropyl-2-((1,3-dioxoisoindolin-2-yl)methyl)imidazo[1,2-a]pyridin-8-yl)azetidine-1-carboxylate (320 mg, 0.68 mmol) in dioxane (10 mL) was added HCl (2 mL, 4 M solution in dioxane, 4 mmol). The resulting mixture was stirred at room temperature for 1 h and then concentrated in vacuo to give the residue, which was washed with saturated aqueous NaHCO₃ (30 mL), extracted with EtOAc (3×30 mL). The combined organic layers were washed with brine (30 mL), dried over Na₂SO₄, concentrated in vacuo to give the crude, which was purified with silica gel chromatography (eluent: DMC/MeOH=15/1) to give 2-((8-(azetidin-3-yl)-6-cyclopropylimidazo[1,2-a]pyridin-2-yl)methyl)isoindoline-1,3-dione (190 mg, 75%) as a yellow solid. ESI-MS [M+H]⁺: 373.1.

### Synthesis of 2-((6-cyclopropyl-8-(1-methylazetidin-3-yl)imidazo[1,2-a]pyridin-2-yl)methyl)isoindoline-1,3-dione

To a solution of 2-((8-(azetidin-3-yl)-6-cyclopropylimidazo[1,2-a]pyridin-2-yl)methyl)isoindoline-1,3-dione (190 mg, 0.51 mmol) in MeOH (10 mL) was added paraformaldehyde (46 mg, 1.53 mmol) and NaBH₃(CN) (97 mg, 1.53 mmol). After stirring the resulting mixture at room temperature for 12 h, water (30 mL) was added and the mixture extracted with EtOAc (3×30 mL). The combined organic layers were washed with brine (30 mL), dried over Na₂SO₄, concentrated in vacuo give the crude, which was purified with silica gel chromatography (eluent: DCM/MeOH=20/1) to give 2-((6-cyclopropyl-8-(1-methylazetidin-3-yl)imidazo[1,2-a]pyridin-2-yl)methyl)isoindoline-1,3-dione (140 mg, 71%) as a yellow solid. ESI-MS [M+H]⁺: 387.2.

### Synthesis of (6-cyclopropyl-8-(1-methylazetidin-3-yl)imidazo[1,2-a]pyridin-2-yl)methanamine

A solution of 2-((6-cyclopropyl-8-(1-methylazetidin-3-yl)imidazo[1,2-a]pyridin-2-yl)methyl)isoindoline-1,3-dione (140 mg, 0.36 mmol) in NH₂NH₂—H₂O (0.5 mL) in EtOH (10 mL) was stirred at 85° C. for 2 h. After cooling to the room temperature, the white solid was precipitated and filtered. The filtrate was concentrated in vacuo to give (6-cyclopropyl-8-(1-methylazetidin-3-yl)imidazo[1,2-a]pyridin-2-yl)methanamine (80 mg crude) as yellow oil, which was used into next step without further purification. ESI-MS [M+H]⁺: 257.2.

### Synthesis of tert-butyl 3-(2-(aminomethyl)-6-cyclopropylimidazo[1,2-a]pyridin-8-yl)azetidine-1-carboxylate

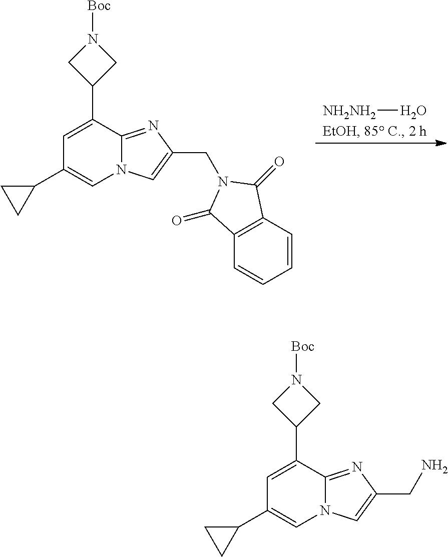

To a solution of tert-butyl 3-(6-cyclopropyl-2-((1,3-dioxoisoindolin-2-yl)methyl)imidazo[1,2-a]pyridin-8-yl)azetidine-1-carboxylate (150 mg, 0.32 mmol) in NH₂NH₂—H₂O (0.5 mL) in EtOH (10 mL) was stirred at 85° C. for 2 h. After cooling to the room temperature, the white solid was precipitated and filtered. The filtrate was concentrated in vacuo to give tert-butyl 3-(2-(aminomethyl)-6-cyclopropylimidazo[1,2-a]pyridin-8-yl)azetidine-1-carboxylate (100 mg crude) as a yellow solid, which was used without further purification. ESI-MS [M+H]⁺: 343.2.

### Synthesis of (6-cyclopropyl-8-(4-(oxetan-3-yl)piperazin-1-yl)imidazo[1,2-a]pyridin-2-yl)methanamine

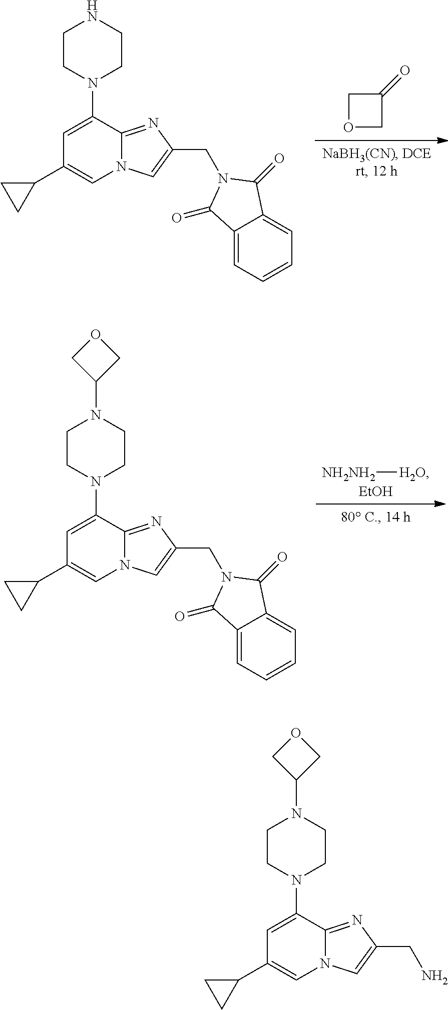

### Synthesis of 2-((6-cyclopropyl-8-(4-(oxetan-3-yl)piperazin-1-yl)imidazo[1,2-a]pyridin-2yl)methyl)isoindoline-1,3-dione

To a mixture of 2-((6-cyclopropyl-8-(piperazin-1-yl)imidazo[1,2-a]pyridin-2-yl)methyl)isoindoline-1,3-dione (50 mg, 0.12 mmol) and oxetan-3-one (17 mg, 0.24 mmol) in DCE (10 mL) was added NaBH₃CN (23 mg, 0.37 mmol) slowly at 0° C. The reaction mixture was stirred at room temperature for 12 h. The resulting mixture was quenched with water (20 mL) and the aqueous phase was extracted with EtOAc (3×20 mL). The combined organic layers were washed with brine (30 mL), dried over Na₂SO₄ and concentrated in vacuo to give the crude, which was purified by Pre-TLC (DCM/MeOH=10/1) to give 2-((6-cyclopropyl-8-(4-(oxetan-3-yl)piperazin-1-yl)imidazo[1,2-a]pyridin-2-yl)methyl)isoindoline-1,3-dione (30 mg, 54%) as a yellow oil.

ESI-MS [M+H]+: 458.2

### Synthesis of (6-cyclopropyl-8-(4-(oxetan-3-yl)piperazin-1-yl)imidazo[1,2-a]pyridin-2-yl)methanamine

To a solution of 2-((6-cyclopropyl-8-(4-(oxetan-3-yl)piperazin-1-yl)imidazo[1,2-a]pyridin-2-yl)methyl)isoindoline-1,3-dione (30 mg, 0.066 mmol)) in EtOH (2 mL) was added NH₂NH₂—H₂O (0.2 mL). The reaction mixture was stirred at 85° C. for 14 h. The resulting mixture was filtered, and the filtrate was concentrated in vacuo to give (6-cyclopropyl-8-(4-(oxetan-3-yl)piperazin-1-yl)imidazo[1,2-a]pyridin-2-yl)methanamine (200 mg, crude) as a yellow oil, which was used in next step without purification. ESI-MS [M+H]+: 328.2

### Synthesis of tert-butyl (6-cyclopropyl-2-((1,3-dioxoisoindolin-2-yl)methyl)imidazo[1,2-a]pyridin-8-yl)carbamate

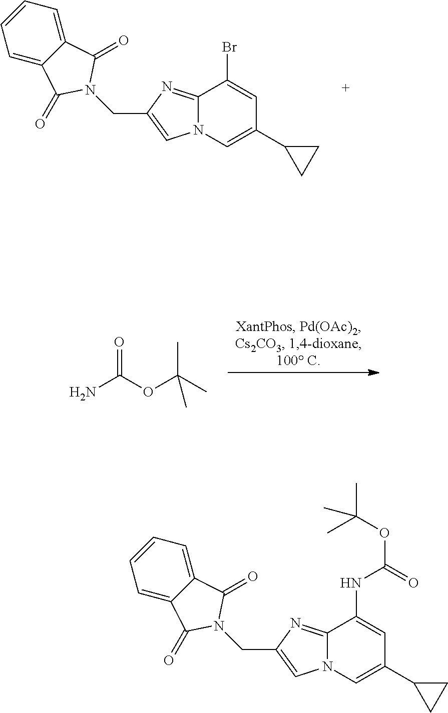

tert-Butyl carbamate (330 mg, 2.8 mmol), 2-((8-bromo-6-cyclopropylimidazo[1,2-a]pyridin-2-yl)methyl)isoindoline-1,3-dione (1000 mg, 2.5 mmol), XantPhos (29 mg, 0.500 mmol), and Cs₂CO₃ (1600 mg, 5.1 mmol) were dissolved in 1,4-dioxane (15 mL) and degassed with nitrogen for 10 min. Pd(OAc)₂ (57 mg, 0.25 mmol) was added and the reaction mixture was heated in a microwave at 130° C. for 3 h. The reaction mixture was cooled to room temperature, filtered through Celite®, washed with methanol and concentrated in vacuo. The residue was purified by silica gel chromatography eluting with a gradient of 30-60% EtOAc in cyclohexane to give the title compound (704 mg, 65%).

ESI-MS (M+H)+: 433.2, ¹H NMR (400 MHz, CDCl₃) δ 7.88-7.86 (m, 2H), 7.73-7.70 (m, 3H), 7.58 (s, 1H), 7.47-7.45 (m, 2H), 4.99 (s, 2H), 1.53 (s, 9H), 0.91-0.86 (m, 2H), 0.68-0.63 (m, 2H).

### Synthesis of N-(2-(aminomethyl)-6-cyclopropylimidazo[1,2-I]pyridin-8-yl)methanesulfonamide

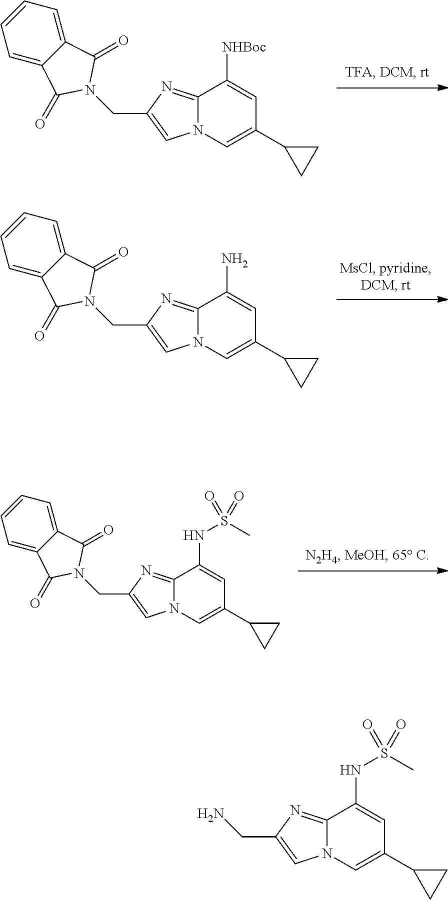

### Synthesis of 2-((8-amino-6-cyclopropylimidazo[1,2-a]pyridin-2-yl)methyl)isoindoline-1,3-dione

Trifluoroacetic acid (0.64 mL, 8.3 mmol) was added to a solution of tert-butyl (6-cyclopropyl-2-((1,3-dioxoisoindolin-2-yl)methyl)imidazo[1,2-a]pyridin-8-yl)carbamate (900 mg, 2.1 mmol) in DCM (10 mL). The reaction mixture was stirred at room temperature for 6 h. The reaction mixture was quenched with Na₂CO₃ (sat·aq·, 20 mL) and extracted with DCM (3×75 mL). The combined organics were dried over a hydrophobic frit and concentrated in vacuo to give the title compound (700 mg, quant.) which was used without further purification. ESI-MS (M+H)+: 333.2

### Synthesis of N-(6-cyclopropyl-2-((1,3-dioxoisoindolin-2-yl)methyl)imidazo[1,2-a]pyridin-8-yl)methanesulfonamide

Methanesulfonyl chloride (0.16 mL, 2.1 mmol) was added to a solution of 2-((8-amino-6-cyclopropylimidazo[1,2-a]pyridin-2-yl)methyl)isoindoline-1,3-dione (700 mg, 2.1 mmol) and pyridine (0.51 mL, 6.3 mmol) in DCM (0.3 mL) was stirred at room temperature for 16 h. The mixture was quenched with water (5 mL) and extracted with DCM (3×25 mL). The organics were dried over a hydrophobic frit and concentrated in vacuo to give the title compound (820 mg, 95%). This was used without further purification.

### Synthesis of N-(2-(aminomethyl)-6-cyclopropylimidazo[1,2-a]pyridin-8-yl)methanesulfonamide

Using a similar procedure to that for 3-(2-(aminomethyl)-6-cyclopropylimidazo[1,2-a]pyridin-8-yl)oxetan-3-ol, N-(2-(aminomethyl)-6-cyclopropylimidazo[1,2-a]pyridin-8-yl)methanesulfonamide (260 mg, 95%) was synthesised from N-(6-cyclopropyl-2-((1,3-dioxoisoindolin-2-yl)methyl)imidazo[1,2-a]pyridin-8-yl)methanesulfonamide (400 mg, 0.975 mmol) using hydrazine hydrate. ESI-MS (M+H)+: 281.1

### Synthesis of N-(2-(((6-chloropyrimidin-4-yl)amino)methyl)-6-cyclopropylimidazo[1,2-a]pyridin-8-yl)-N-methylacetamide

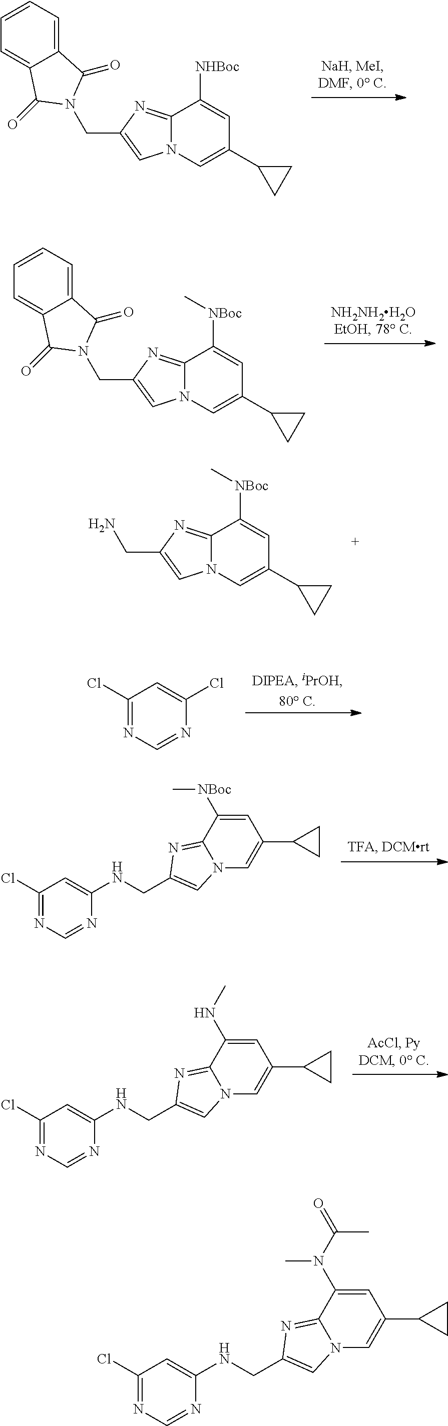

### Synthesis of tert-butyl (6-cyclopropyl-2-((1,3-dioxoisoindolin-2-yl)methyl)imidazo[1,2-a]pyridin-8-yl)(methyl)carbamate

NaH (60% in mineral oil, 55.5 mg, 1.39 mmol) was added to stirred solution of tert-butyl (6-cyclopropyl-2-((1,3-dioxoisoindolin-2-yl)methyl)imidazo[1,2-a]pyridin-8-yl)carbamate (500 mg, 1.16 mmol) in DMF (10 mL) at 0° C. The reaction was stirred at 0° C. for 30 min. MeI (86 μL, 1.39 mmol) was then added and the reaction stirred at 0° C. for 2 h. The reaction was quenched with water (15 mL) and extracted with EtOAc (3×15 mL). The organics were combined, dried over MgSO₄ and concentrated in vacuo. The residue was purified by silica gel chromatography, eluting with a gradient of 0-100% EtOAc in cyclohexane to give the title compound (350 mg, 68%). ¹H NMR (400 MHz, CDCl₃) δ 7.88 (dd, J=3.0, 5.3 Hz, 2H), 7.73 (dd, J=3.0, 5.6 Hz, 2H), 7.68 (s, 1H), 7.40 (s, 1H), 6.83-6.78 (m, 1H), 5.06 (s, 2H), 3.34 (s, 3H), 1.88-1.80 (m, 1H), 1.37 (s, 9H), 0.95-0.89 (m, 2H), 0.64-0.59 (m, 2H).

### Synthesis of tert-butyl (2-(aminomethyl)-6-cyclopropylimidazo[1,2-a]pyridin-8-yl)(methyl)carbamate

Using a similar procedure to that for 3-(2-(aminomethyl)-6-cyclopropylimidazo[1,2-a]pyridin-8-yl)oxetan-3-ol, tert-butyl (2-(aminomethyl)-6-cyclopropylimidazo[1,2-a]pyridin-8-yl)(methyl)carbamate (260 mg) was synthesised from tert-butyl (6-cyclopropyl-2-((1,3-dioxoisoindolin-2-yl)methyl)imidazo[1,2-a]pyridin-8-yl)(methyl)carbamate (350 mg, 0.79 mmol) using hydrazine hydrate. ESI-MS (M+H)+: 317.4

### Synthesis of tert-butyl (2-(((6-chloropyrimidin-4-yl)amino)methyl)-6-cyclopropylimidazo[1,2-a]pyridin-8-yl)(methyl)carbamate

DIPEA (0.44 mL, 2.56 mmol) was added to a stirred solution of 4,6-dichloropyrimidine (150 mg, 0.85 mmol) tert-butyl (2-(aminomethyl)-6-cyclopropylimidazo[1,2-a]pyridin-8-yl)(methyl)carbamate (260 mg, 0.85 mmol) in isopropanol (10 mL). The reaction was then heated at 80° C. for 18 h. The reaction was cooled to room temperature and partitioned between water (30 mL) and EtOAc (30 mL), the layers were separated and the aqueous layer was further extracted with EtOAc (2×20 mL). The organics were combined, dried over MgSO₄ and concentrated in vacuo to give the title compound (160 mg, 40%). ESI-MS (M+H)+: 429.3, ¹H NMR (400 MHz, CDCl₃) δ 8.36 (s, 1H), 7.75 (s, 1H), 7.43 (s, 1H), 6.86 (s, 1H), 6.41 (s, 1H), 4.70-4.60 (m, 2H), 3.35 (s, 3H), 1.92-1.83 (m, 1H), 1.42 (s, 9H), 1.00-0.92 (m, 2H), 0.69-0.62 (m, 2H). One exchangeable (NH) not visible.

### Synthesis of 2-(((6-chloropyrimidin-4-yl)amino)methyl)-6-cyclopropyl-N-methylimidazo[1,2-a]pyridin-8-amine

TFA (0.114 mL, 1.49 mmol) was added to a stirred solution of tert-butyl (2-(((6-chloropyrimidin-4-yl)amino)methyl)-6-cyclopropylimidazo[1,2-a]pyridin-8-yl)(methyl)carbamate (160 mg, 0.373 mmol) in DCM (5.0 mL) and stirred at room temperature for 2 h. The reaction was quenched with NaHCO₃ solution (sat·aq·, 20 mL) and extracted with DCM (3×20 mL), the organics were combined, dried over MgSO₄ and concentrated in vacuo to give the title compound (120 mg, 98%). ESI-MS (M+H)+: 329.3

### Synthesis of N-(2-(((6-chloropyrimidin-4-yl)amino)methyl)-6-cyclopropylimidazo[1,2-a]pyridin-8-yl)-N-methylacetamide

Acetyl chloride (26 μL, 0.36 mmol) was added to a stirred solution of 2-(((6-chloropyrimidin-4-yl)amino)methyl)-6-cyclopropyl-N-methylimidazo[1,2-a]pyridin-8-amine (120 mg, 0.36 mmol) and pyridine (88 μL, 1.09 mmol) in DCM (1.0 mL) at 0° C. The reaction was stirred at 0° C. for 1 hour. The reaction was diluted with water (10 mL) and extracted with DCM (3×10 mL). The organics were combined, dried over MgSO₄ and concentrated in vacuo. The residue was purified by silica gel chromatography, eluting with a gradient of 0-10% (7N NH₃ in MeOH) in DCM to give the title compound (100 mg, 74%). ESI-MS (M+H)+: 371.3, ¹H NMR (400 MHz, CDCl₃) δ 8.36 (s, 1H), 7.88 (s, 1H), 7.55 (s, 1H), 6.86 (s, 1H), 6.62 (s, 1H), 6.44 (s, 1H), 4.75-4.63 (m, 2H), 3.33 (s, 3H), 1.97-1.81 (m, 4H), 1.01 (d, J=8.1 Hz, 2H), 0.72-0.64 (m, 2H).

### Synthesis of N-(2-(aminomethyl)-6-cyclopropylimidazo[1,2-a]pyridin-8-yl)-N-methylmethanesulfonamide

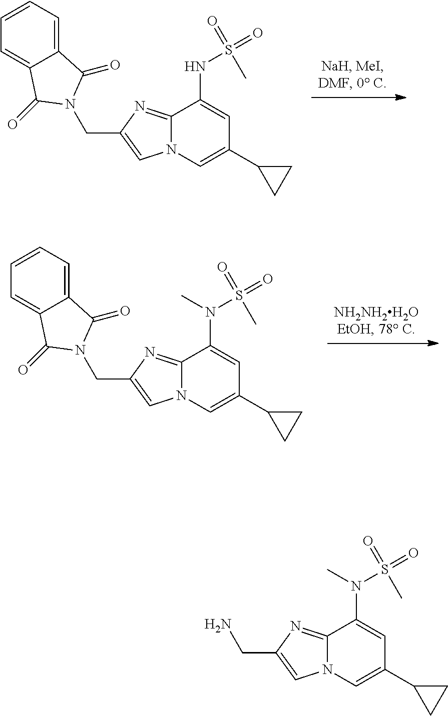 [SMILES: CS(=O)(=O)Nc1cc(C2CC2)cn2cc(CN3C(=O)c4ccccc4C3=O)nc12 ; "NaH, MeI,DMF, 0° C." ; CN(c1cc(C2CC2)cn2cc(CN3C(=O)c4ccccc4C3=O)nc12)S(C)(=O)=O ; "NH2NH2·H2OEtOH, 78° C." ; CN(c1cc(C2CC2)cn2cc(CN)nc12)S(C)(=O)=O]

### Synthesis of N-(6-cyclopropyl-2-((1,3-dioxoisoindolin-2-yl)methyl)imidazo[1,2-a]pyridin-8-yl)-N-methylmethanesulfonamide

NaH (60% in mineral oil, 23.4 mg, 0.59 mmol) was added to stirred solution of N-(6-cyclopropyl-2-((1,3-dioxoisoindolin-2-yl)methyl)imidazo[1,2-a]pyridin-8-yl)methanesulfonamide (200 mg, 0.49 mmol) in DMF (5 mL) at 0° C. The reaction was stirred at 0° C. for 30 min. MeI (36 μL, 0.59 mmol) was then added and the reaction stirred at 0° C. for 4 h. The reaction was quenched with water (15 mL) and extracted with EtOAc (3×15 mL). The organics were combined, dried over MgSO₄ and concentrated in vacuo. The residue was purified by silica gel chromatography, eluting with a gradient of 0-100% EtOAc in cyclohexane to give the title compound (177 mg, 86%). ESI-MS (M+H)+: 425.1, ¹H NMR (400 MHz, CDCl₃) δ 7.86 (dd, J=3.0, 5.6 Hz, 2H), 7.79 (d, J=1.0 Hz, 1H), 7.73 (dd, J=3.2, 5.4 Hz, 2H), 7.49 (s, 1H), 7.01 (d, J=1.5 Hz, 1H), 5.02 (s, 2H), 3.40 (s, 3H), 3.01 (s, 3H), 1.89-1.80 (m, 1H), 0.97-0.91 (m, 2H), 0.67-0.63 (m, 2H).

### Synthesis of N-(2-(aminomethyl)-6-cyclopropylimidazo[1,2-a]pyridin-8-yl)-N-methylmethanesulfonamide

Using a similar procedure to that for 3-(2-(aminomethyl)-6-cyclopropylimidazo[1,2-a]pyridin-8-yl)oxetan-3-ol, N-(2-(aminomethyl)-6-cyclopropylimidazo[1,2-a]pyridin-8-yl)-N-methylmethanesulfonamide (100 mg, 81%) was synthesised from N-(6-cyclopropyl-2-((1,3-dioxoisoindolin-2-yl)methyl)imidazo[1,2-a]pyridin-8-yl)-N-methylmethanesulfonamide (177 mg, 0.42 mmol) using hydrazine hydrate. ESI-MS (M+H)+: 295.1

### Synthesis of (6-cyclopropyl-8-(4H-1,2,4-triazol-4-yl)imidazo[1,2-a]pyridin-2-yl)methanamine

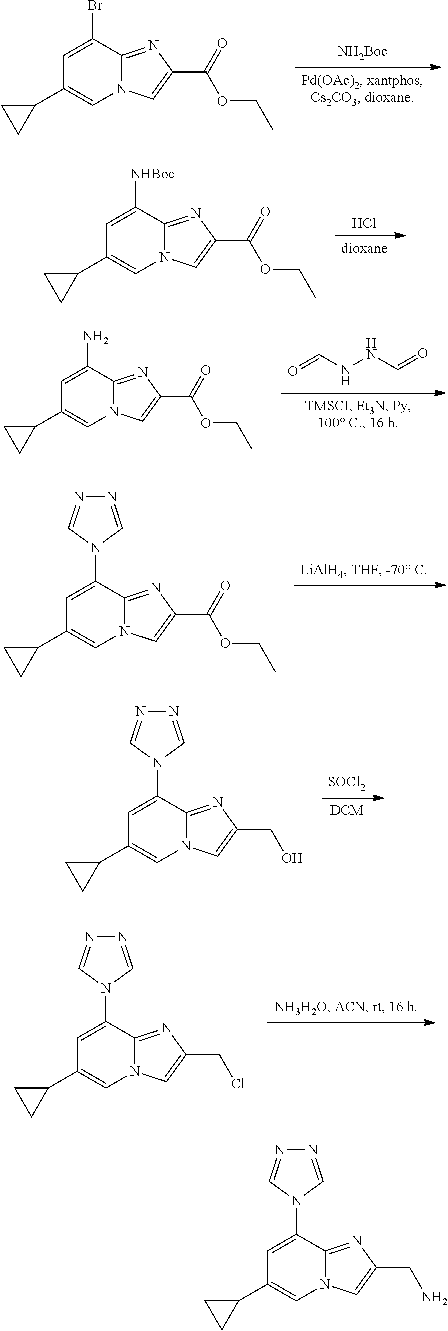

### Synthesis of ethyl 8-((tert-butoxycarbonyl)amino)-6-cyclopropylimidazo[1,2-a]pyridine-2-carboxylate

To a mixture of ethyl 8-bromo-6-cyclopropylimidazo[1,2-a]pyridine-2-carboxylate (1.3 g, 4.22 mmol), tert-butyl carbamate (740.7 mg, 6.33 mmol) and Cs₂CO₃ (3.4 g, 10.55 mmol) in dioxane (20 mL) was added Pd(OAc)₂ (94.5 mg, 0.42 mmol) and Xantphos (159.5 g, 0.42 mmol). The reaction mixture was stirred at 95° C. for 16 h under N₂. The reaction mixture was cooled to room temperature, then filtered through celite and the filter cake was washed with DCM/MeOH (10/1, 50 mL). The filtrate was concentrated in vacuo to give the crude, which was purified by silica gel chromatography (eluent: DCM/MeOH=40/1) to give ethyl 8-((tert-butoxycarbonyl)amino)-6-cyclopropylimidazo[1,2-a]pyridine-2-carboxylate as a yellow solid (1.3 g, 89%). ESI-MS [M+H]⁺: 346.2.

### Synthesis of ethyl 8-amino-6-cyclopropylimidazo[1,2-a]pyridine-2-carboxylate

A solution of ethyl 8-((tert-butoxycarbonyl)amino)-6-cyclopropylimidazo[1,2-a]pyridine-2-carboxylate (1.3 g, 3.77 mmol) in HCl (4M in dioxane, 30 mL) was stirred at room temperature for 2 h. The mixture was concentrated in vacuo to give ethyl 8-amino-6-cyclopropylimidazo[1,2-a]pyridine-2-carboxylate (924 mg, crude) as a yellow solid, which was used for the next step directly without purification. ESI-MS [M+H]⁺: 246.1.

### Synthesis of ethyl 6-cyclopropyl-8-(4H-1,2,4-triazol-4-yl)imidazo[1,2-a]pyridine-2-carboxylate

To a solution of ethyl 8-amino-6-cyclopropylimidazo[1,2-a]pyridine-2-carboxylate (924 mg, crude), N′-formylformohydrazide (995.3 mg, 11.31 mmol) and Et₃N (2.66 g, 26.39 mmol) in pyridine (20 mL) was added TMSCl (2.85 g, 26.39 mmol) at 0° C. Then the mixture was stirred at 100° C. for 16 h under N₂. After cooling to room temperature, the mixture was concentrated in vacuo to get the crude product, which was purified by silica gel chromatography (eluent: DCM/MeOH=50/1˜40/1) to give ethyl 6-cyclopropyl-8-(4H-1,2,4-triazol-4-yl)imidazo[1,2-a]pyridine-2-carboxylate as a yellow solid (500 mg, 44%). ESI-MS [M+H]⁺: 298.2.

### Synthesis of (6-cyclopropyl-8-(4H-1,2,4-triazol-4-yl)imidazo[1,2-a]pyridin-2-yl)methanol

To a solution of ethyl 6-cyclopropyl-8-(4H-1,2,4-triazol-4-yl)imidazo[1,2-a]pyridine-2-carboxylate (500 mg, 1.68 mmol) in THF (20.0 mL) was stirred LiAlH₄ (8.4 mL, 8.4 mmol) at −70° C. under N₂. After stirring at −70° C. for 3 h, the reaction mixture was quenched with water (20 mL) at −70° C., warmed to room temperature and stirred for 10 min. The suspension was filtered and the filtrate was concentrated in vacuo and purified by preparative TLC (eluent: DCM/MeOH=50/1˜40/1) to give (6-cyclopropyl-8-(4H-1,2,4-triazol-4-yl)imidazo[1,2-a]pyridin-2-yl)methanol (120 mg, 28%) as a light yellow solid. ESI-MS [M+H]+: 256.1.

### Synthesis of 2-(chloromethyl)-6-cyclopropyl-8-(4H-1,2,4-triazol-4-yl)imidazo[1,2-a]pyridine

To a solution of (6-cyclopropyl-8-(4H-1,2,4-triazol-4-yl)imidazo[1,2-a]pyridin-2-yl)methanol (120 mg, 0.47 mmol) in DCM (10.0 mL) was added SOCl₂ (1.0 mL) at 0° C. and it was stirred at room temperature for 1 h. Then the reaction mixture was concentrated in vacuo to give 2-(chloromethyl)-6-cyclopropyl-8-(4H-1,2,4-triazol-4-yl)imidazo[1,2-a]pyridine (120 mg, crude) as a yellow oil, which was used for the next step directly without purification. ESI-MS [M+H]+: 274.1.

### Synthesis of (6-cyclopropyl-8-(4H-1,2,4-triazol-4-yl)imidazo[1,2-a]pyridin-2-yl)methanamine

To a solution of 2-(chloromethyl)-6-cyclopropyl-8-(4H-1,2,4-triazol-4-yl)imidazo[1,2-a]pyridine (120 mg, 0.44 mmol) in CH₃CN (2.0 mL) was added NH₃ (25 wt % in water, 2.0 mL) at room temperature and stirred for 16 h. The mixture was then concentrated to give (6-cyclopropyl-8-(4H-1,2,4-triazol-4-yl)imidazo[1,2-a]pyridin-2-yl)methanamine (100 mg, crude) as a yellow oil. which was used for the next step without purification. ESI-MS [M+H]+: 255.1.

### Synthesis of 3-(2-(aminomethyl)-6-cyclopropylimidazo[1,2-a]pyridin-8-yl)oxazolidin-2-one

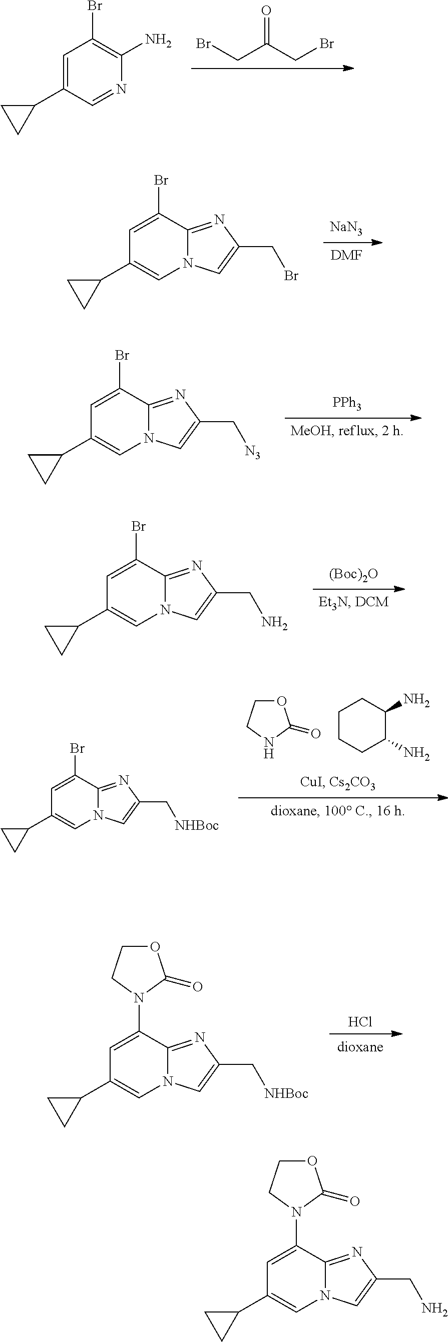

### Synthesis of 8-bromo-2-(bromomethyl)-6-cyclopropylimidazo[1,2-a]pyridine

A mixture of 3-bromo-5-cyclopropylpyridin-2-amine (1.06 g, 5.0 mmol) and 1,3-dibromopropan-2-one (1.62 g, 7.5 mmol) in DME (20.0 mL) was stirred at 90° C. under N₂ for 16 h. The reaction mixture was warmed to room temperature, quenched with sat·NaHCO₃ solution (50 mL) and extracted with EtOAc (3×50 ml). The combined organics were washed with brine (50 mL), dried over Na₂SO₄ and concentrated in vacuo. The crude residue was purified by column chromatography (eluent: DCM/MeOH=50/1˜30/1) to give the product 8-bromo-2-(bromomethyl)-6-cyclopropylimidazo[1,2-a]pyridine (1.2 g, 73%) as a yellow solid. ESI-MS [M+H]⁺: 330.2.

### Synthesis of 2-(azidomethyl)-8-bromo-6-cyclopropylimidazo[1,2-a]pyridine

A mixture of 8-bromo-2-(bromomethyl)-6-cyclopropylimidazo[1,2-a]pyridine (1.0 g, 3.0 mmol) and NaN₃ (244 mg, 3.75 mmol) in DMF (10.0 mL) was stirred at room temperature under N₂ for 16 h. The mixture was diluted with EtOAc (100 mL) and washed with brine (3×50 mL). The organic layer was dried over Na₂SO₄ and concentrated in vacuo to give 2-(azidomethyl)-8-bromo-6-cyclopropylimidazo[1,2-a]pyridine (900 mg, crude) as a yellow solid which was used for the next step without purification. ESI-MS [M+H]⁺: 292.2.

### Synthesis of (8-bromo-6-cyclopropylimidazo[1,2-a]pyridin-2-yl)methanamine

A mixture of 2-(azidomethyl)-8-bromo-6-cyclopropylimidazo[1,2-a]pyridine (870 mg, 3.0 mmol) and PPh₃ (983 mg, 3.75 mmol) in MeOH (25 mL) was stirred at reflux for 2 h. The mixture was concentrated in vacuo and purified by preparative TLC (eluent: DCM/MeOH=10/1) to give (8-bromo-6-cyclopropylimidazo[1,2-a]pyridin-2-yl)methanamine (550 mg, 69%) as a yellow oil. ESI-MS [M+H]⁺: 266.2.

### Synthesis of tert-butyl ((8-bromo-6-cyclopropylimidazo[1,2-a]pyridin-2-yl)methyl)carbamate

A mixture of (8-bromo-6-cyclopropylimidazo[1,2-a]pyridin-2-yl)methanamine (550 mg, 2.08 mmol), (Boc)₂O (679 mg, 3.11 mmol) and Et₃N (630 mg, 6.24 mmol) in DCM (20 mL) was stirred at room temperature for 16 h. The mixture was quenched with water (50 mL) and extracted with DCM (3×50 ml). The combined organics were washed with brine (50 mL), dried over Na₂SO₄ and concentrated in vacuo. The residue was purified by preparative TLC (eluent: DCM/MeOH=20/1) to give tert-butyl ((8-bromo-6-cyclopropylimidazo[1,2-a]pyridin-2-yl)methyl)carbamate (520 mg, 69%) as a yellow solid. ESI-MS [M+H]⁺: 366.2.

### Synthesis of tert-butyl ((6-cyclopropyl-8-(2-oxooxazolidin-3-yl)imidazo[1,2-a]pyridin-2-yl)methyl)carbamate

A mixture of tert-butyl ((8-bromo-6-cyclopropylimidazo[1,2-a]pyridin-2-yl)methyl)carbamate (100 mg, 0.27 mmol), oxazolidin-2-one (47.7 mg, 0.55 mmol), (1R,2R)-cyclohexane-1,2-diamine (30.8 mg, 0.27 mmol), CuI (51.3 mg, 0.27 mmol) and Cs₂CO₃ (179.3 mg, 0.55 mmol) in dioxane (10 mL) was stirred at 100° C. under N₂ for 16 h. The reaction mixture was allowed to reach room temperature, quenched with water (50 mL) and extracted with DCM (3×50 ml). The combined organics were washed with brine (50 mL), dried over Na₂SO₄ and concentrated in vacuo. The residue was purified by preparative TLC (eluent: DCM/MeOH=10/1) to give the product tert-butyl ((6-cyclopropyl-8-(2-oxooxazolidin-3-yl)imidazo[1,2-a]pyridin-2-yl)methyl)carbamate (50 mg, 50%) as a light yellow solid. ESI-MS [M+H]⁺: 373.2.

### Synthesis of 3-(2-(aminomethyl)-6-cyclopropylimidazo[1,2-a]pyridin-8-yl)oxazolidin-2-one

A mixture of tert-butyl ((6-cyclopropyl-8-(2-oxooxazolidin-3-yl)imidazo[1,2-a]pyridin-2-yl)methyl)carbamate (50 mg, 0.13 mmol) in HCl/dioxane (4M, 5.0 mL) was stirred at room temperature for 2 h. The resulting solution was concentrated to give 3-(2-(aminomethyl)-6-cyclopropylimidazo[1,2-a]pyridin-8-yl)oxazolidin-2-one (35 mg, crude) as a yellow solid which was used for the next step directly. ESI-MS [M+H]⁺: 273.2.

Using a similar procedure to that used for 3-(2-(aminomethyl)-6-cyclopropylimidazo[1,2-a]pyridin-8-yl)oxazolidin-2-one, the compounds in Table A8 were prepared from tert-butyl ((8-bromo-6-cyclopropylimidazo[1,2-a]pyridin-2-yl)methyl)carbamate and an appropriate coupling partner, followed by treatment with HCl in dioxane.

TABLE A8

| Structure | Coupling Partner | Analytical Data |
|---|---|---|
| 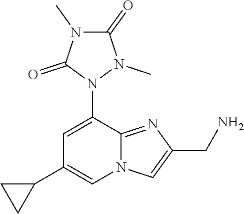 [SMILES: Cn1c(=O)n(C)n(-c2cc(C3CC3)cn3cc(CN)nc23)c1=O] | 1,4-dimethyl-1,2,4-triazolidine- 3,5-dione | ESI-MS [M + H]+: 315.2 |
| 1-(2-(aminomethyl)-6- | | |
| cyclopropylimidazo[1,2-a]pyridin-8-yl)- | | |
| 2,4-dimethyl-1,2,4-triazolidine-3,5-dione | | |
| hydrochloric acid salt | | |
| 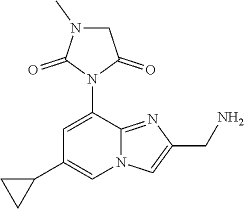 [SMILES: CN1CC(=O)N(c2cc(C3CC3)cn3cc(CN)nc23)C1=O] | 1-methylimidazolidine-2,4- dione | ESI-MS [M + H]+: 300.1 |
| 3-(2-(aminomethyl)-6- | | |
| cyclopropylimidazo[1,2-a]pyridin-8-yl)-1- | | |
| methylimidazolidine-2,4-dione | | |

### Synthesis of 1-(2-(aminomethyl)-6-cyclopropylimidazo[1,2-a]pyridin-8-yl)-4-methylpiperazine-2,5-dione hydrochloride

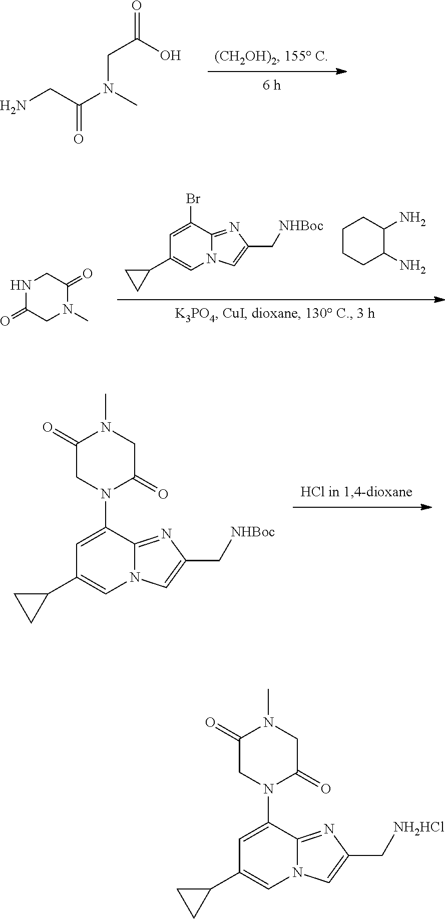

### Synthesis of 1-methylpiperazine-2,5-dione

A solution of N-glycyl-N-methylglycine (1.1 g, 7.53 mmol) in (CH₂OH)₂ (10 mL) was stirred at 155° C. for 6 h. The reaction was cooled to room temperature and concentrated in vacuo to give the crude, which was purified by silica gel chromatography (eluent: DCM/MeOH=25/1) to give 1-methylpiperazine-2,5-dione (800 mg, 83%) as a white solid. ESI-MS: [M+H]+, 129.2.

### Synthesis of tert-butyl ((6-cyclopropyl-8-(4-methyl-2,5-dioxopiperazin-1-yl)imidazo[1,2-a]pyridin-2-yl)methyl)carbamate

A mixture of tert-butyl ((8-bromo-6-cyclopropylimidazo[1,2-a]pyridin-2-yl)methyl)carbamate (200 mg, 0.55 mmol), 1-methylpiperazine-2,5-dione (210 mg, 1.64 mmol), cyclohexane-1,2-diamine (31 mg, 0.27 mmol), K₃PO₄ (232 mg, 1.09 mmol) and CuI (21 mg, 0.11 mmol) in 1,4-dioxane (10 ml) was stirred at 130° C. for 3 h. The reaction mixture was cooled to room temperature, filtered through celite and the filter cake was washed with DCM (60 mL). The filtrate was concentrated in vacuo to give the crude, which was purified by silica gel chromatography (eluent: DCM/MeOH=100/7) to give tert-butyl ((6-cyclopropyl-8-(4-methyl-2,5-dioxopiperazin-1-yl)imidazo[1,2-a]pyridin-2-yl)methyl)carbamate (60 mg, 26%) as a yellow solid. ESI-MS: [M+H]⁺, 314.2

### Synthesis of 1-(2-(aminomethyl)-6-cyclopropylimidazo[1,2-a]pyridin-8-yl)-4-methylpiperazine-2,5-dione hydrochloride

To a solution of tert-butyl ((6-cyclopropyl-8-(4-methyl-2,5-dioxopiperazin-1-yl)imidazo[1,2-a]pyridin-2-yl)methyl)carbamate (50 mg, 0.12 mmol) in 1,4-dioxane (1 ml) was added HCl (4 M solution in dioxane, 1.5 ml, 6 mmol). The resulting mixture was stirred at room temperature for 1 h and concentrated in vacuo to give 11-(2-(aminomethyl)-6-cyclopropylimidazo[1,2-a]pyridin-8-yl)-4-methylpiperazine-2,5-dione hydrochloride (60 mg, 100%) which was used to the next step without further purification. ESI-MS: [M+H]+, 314.2

### Synthesis of 1-(2-(aminomethyl)-6-cyclopropylimidazo[1,2-a]pyridin-8-yl)-3-methylimidazolidine-2,4-dione

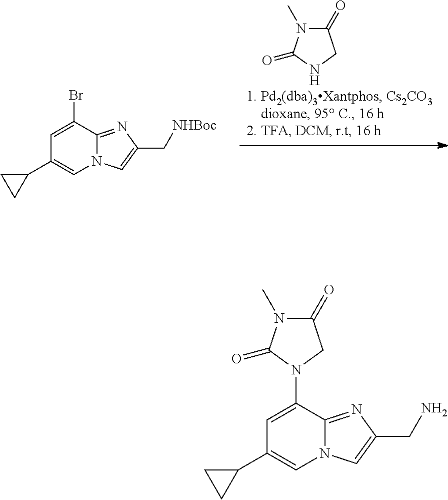

### Synthesis of tert-butyl ((6-cyclopropyl-8-(3-methyl-2,4-dioxoimidazolidin-1-yl)imidazo[1,2-a]pyridin-2-yl)methyl)carbamate

To a mixture of 3-methylimidazolidine-2,4-dione (224 mg, 2 mmol), tert-butyl ((8-bromo-6-cyclopropylimidazo[1,2-a]pyridin-2-yl)methyl)carbamate (365 mg, 1 mmol) and Cs₂CO₃ (975 mg, 3 mmol) in dry 1,4-dioxane (10 mL) was added Pd₂(dba)₃ (183 mg, 0.2 mmol) and XantPhos (116 mg, 0.2 mmol). The reaction was stirred at 100° C. for 16 h under N₂, quenched with water (50 mL) and extracted with EtOAc (30 mL×3). The organic layers were washed with brine (30 mL), dried with Na₂SO₄, concentrated in vacuo and purified by silica gel chromatography (eluent: DCM/MeOH=10/1) to give tert-butyl ((6-cyclopropyl-8-(3-methyl-2,4-dioxoimidazolidin-1-yl)imidazo[1,2-a]pyridin-2-yl)methyl)carbamate (150 mg, 37%) as a yellow solid. ESI-MS [M+H]⁺: 400.0.

### Synthesis of 1-(2-(aminomethyl)-6-cyclopropylimidazo[1,2-a]pyridin-8-yl)-3-methylimidazolidine-2,4-dione

To a mixture of tert-butyl ((6-cyclopropyl-8-(3-methyl-2,4-dioxoimidazolidin-1-yl)imidazo[1,2-a]pyridin-2-yl)methyl)carbamate (500 mg, 1.25 mmol) in dry DCM (10 mL) was added TFA (2 ml). The reaction mixture was stirred at room temperature for 16 h under N₂ and then neutralized with methanolic ammonia solution (7M). The resulting solution was concentrated in vacuo and purified by silica gel chromatography (eluent: DCM/MeOH=10/1) to give 1-(2-(aminomethyl)-6-cyclopropylimidazo[1,2-a]pyridin-8-yl)-3-methylimidazolidine-2,4-dione (150 mg, 40%) as a white solid. ESI-MS [M+H]⁺: 300.1.

Using a similar procedure to that used for 1-(2-(aminomethyl)-6-cyclopropylimidazo[1,2-a]pyridin-8-yl)-3-methylimidazolidine-2,4-dione, the compounds in Table A9 were prepared from tert-butyl ((8-bromo-6-cyclopropylimidazo[1,2-a]pyridin-2-yl)methyl)carbamate and an appropriate coupling partner, followed by treatment with TFA in dichloromethane or HCl in dioxane.

TABLE A9

| Structure | Coupling Partner | Analytical Data |
|---|---|---|
| 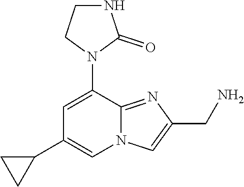 [SMILES: NCc1cn2cc(C3CC3)cc(N3CCNC3=O)c2n1] | imidazolidin-2-one | ESI-MS: [M + H]+, 272.2 |
| 1-(2-(aminomethyl)-6- | | |
| cyclopropylimidazo[1,2-a]pyridin-8- | | |
| yl)imidazolidin-2-one hydrochloride | | |
| 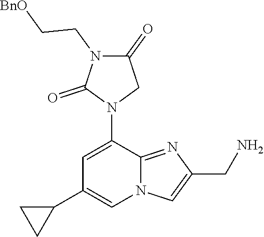 [SMILES: NCc1cn2cc(C3CC3)cc(N3CC(=O)N(CCOCc4ccccc4)C3=O)c2n1] | 3-(2- (benzyloxy)ethyl) imidazolidine-2,4- dione | ESI-MS [M + H]⁺: 420.2 |
| 1-(2-(aminomethyl)-6- | | |
| cyclopropylimidazo[1,2-a]pyridin-8-yl)-3- | | |
| (2-(benzyloxy)ethyl)imidazolidine-2,4- | | |
| dione hydrochloride | | |
| 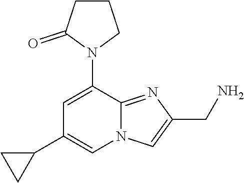 [SMILES: NCc1cn2cc(C3CC3)cc(N3CCCC3=O)c2n1] | pyrrolidin-2-one | ESI-MS [M + H]⁺: 271.1 |
| 1-(2-(aminomethyl)-6- | | |
| cyclopropylimidazo[1,2-a]pyridin-8- | | |
| yl)pyrrolidin-2-one hydrochloride | | |
| 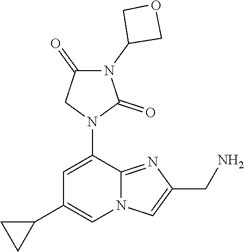 [SMILES: NCc1cn2cc(C3CC3)cc(N3CC(=O)N(C4COC4)C3=O)c2n1] | 3-(oxetan-3- yl)imidazolidine- 2,4-dione | ESI-MS [M + H] +: 342.2 |
| 1-(2-(aminomethyl)-6- | | |
| cyclopropylimidazo[1,2-a]pyridin-8-yl)-3- | | |
| (oxetan-3-yl)imidazolidine-2,4-dione | | |
| 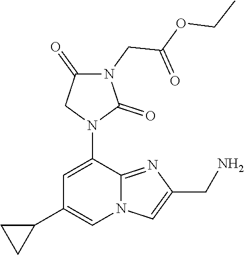 [SMILES: CCOC(=O)CN1C(=O)CN(c2cc(C3CC3)cn3cc(CN)nc23)C1=O] | ethyl 2-(2,5- dioxoimidazolidin- 1-yl)acetate | ESI-MS [M + H] ⁺: 372.2 |
| 2-(3-(2-(aminomethyl)-6- | | |
| ethyl 2-(3-(2-(aminomethyl)-6- | | |
| cyclopropylimidazo[1,2-a]pyridin-8-yl)- | | |
| 2,5-dioxoimidazolidin-1-yl)acetate | | |
| 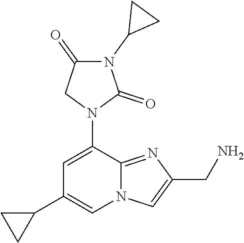 [SMILES: NCc1cn2cc(C3CC3)cc(N3CC(=O)N(C4CC4)C3=O)c2n1] | 3- cyclopropylimidazo- lidine-2,4-dione | ESI-MS [M + H]+: 326.2 |
| 1-(2-(aminomethyl)-6- | | |
| cyclopropylimidazo[1,2-a]pyridin-8-yl)-3- | | |
| cyclopropylimidazolidine-2,4-dione | | |
| hydrochloride | | |
| 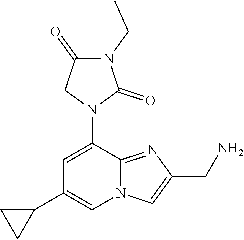 [SMILES: CCN1C(=O)CN(c2cc(C3CC3)cn3cc(CN)nc23)C1=O] | 3- ethylimidazolidine- 2,4-dione | ESI-MS [M + H]⁺: 314.2 |
| 1-(2-(aminomethyl)-6- | | |
| cyclopropylimidazo[1,2-a]pyridin-8-yl)-3- | | |
| ethylimidazolidine-2,4-dione | | |
| hydrochloride | | |
| 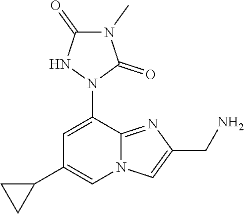 [SMILES: Cn1c(=O)[nH]n(-c2cc(C3CC3)cn3cc(CN)nc23)c1=O] | 4-methyl-1,2,4- triazolidine-3,5- dione | ESI-MS [M + H]+: 301.2 |
| 1-(2-(aminomethyl)-6- | | |
| cyclopropylimidazo[1,2-a]pyridin-8-yl)-4- | | |
| methyl-1,2,4-triazolidine-3,5-dione | | |
| hydrochloride | | |
| 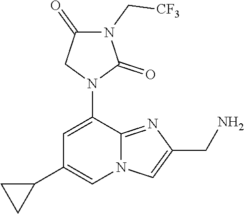 [SMILES: NCc1cn2cc(C3CC3)cc(N3CC(=O)N(CC(F)(F)F)C3=O)c2n1] | 3-(2,2,2- trifluoroethyl) imidazolidine-2,4-dione | ESI-MS [M + H]⁺: 368.2 |
| 1-(2-(aminomethyl)-6- | | |
| cyclopropylimidazo[1,2-a]pyridin-8-yl)-3- | | |
| (2,2,2-trifluoroethyl)imidazolidine-2,4- | | |
| dione | | |
| 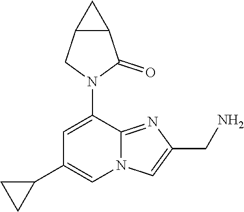 [SMILES: NCc1cn2cc(C3CC3)cc(N3CC4CC4C3=O)c2n1] | 3- azabicyclo[3.1.0] hexan-2-one | ESI-MS [M + H]+: 283.2 |
| 3-(2-(aminomethyl)-6- | | |
| cyclopropylimidazo[1,2-a]pyridin-8-yl)-3- | | |
| azabicyclo[3.1.0]hexan-2-one | | |
| hydrochloride | | |
| 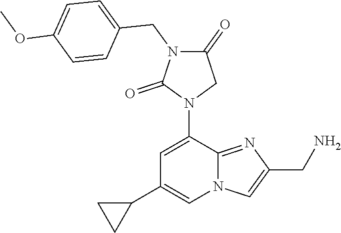 [SMILES: COc1ccc(CN2C(=O)CN(c3cc(C4CC4)cn4cc(CN)nc34)C2=O)cc1] | 3-(4- methoxybenzyl) imidazolidine-2,4-dione | ESI-MS: [M + H]+, 406.2 |
| 1-(2-(aminomethyl)-6- | | |
| cyclopropylimidazo[1,2-a]pyridin-8-yl)-3- | | |
| (4-methoxybenzyl)imidazolidine-2,4- | | |
| dione hydrochloride | | |
| 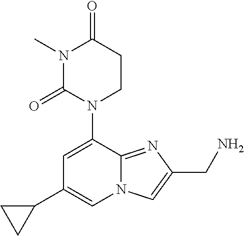 [SMILES: CN1C(=O)CCN(c2cc(C3CC3)cn3cc(CN)nc23)C1=O] | 3- methyldihydropyrimidine- 2,4(1H,3H)-dione | ESI-MS [M + H]+: 314.1 |
| 1-(2-(aminomethyl)-6- | | |
| cyclopropylimidazo[1,2-a]pyridin-8-yl)-3- | | |
| methyldihydropyrimidine-2,4(1H,3H)- | | |
| dione hydrochloride | | |
| 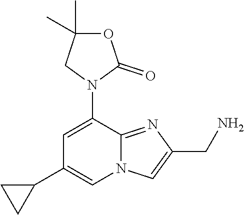 [SMILES: CC1(C)CN(c2cc(C3CC3)cn3cc(CN)nc23)C(=O)O1] | 5,5- dimethyloxazolidin- 2-one | ESI-MS [M + H]+: 301.2 |
| 3-(2-(aminomethyl)-6- | | |
| cyclopropylimidazo[1,2-a]pyridin-8-yl)- | | |
| 5,5-dimethyloxazolidin-2-one | | |
| hydrochloride | | |

### Synthesis of 1-(2-(aminomethyl)-6-cyclopropyl-3-fluoroimidazo[1,2-a]pyridin-8-yl)-3-methylimidazolidine-2,4-dione

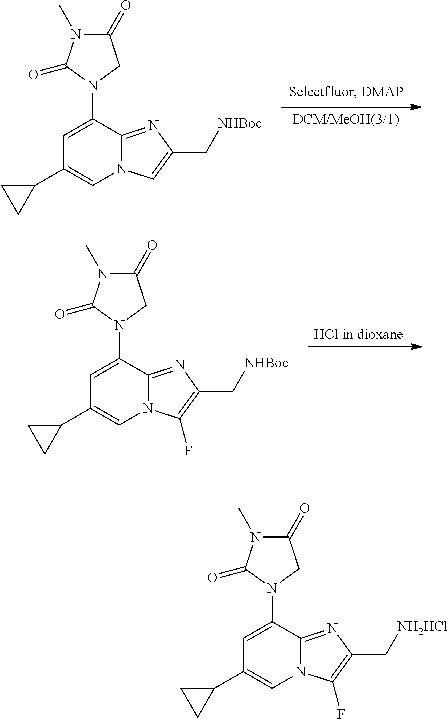

### Synthesis of tert-butyl ((6-cyclopropyl-3-fluoro-8-(3-methyl-2,4-dioxoimidazolidin-1-yl)imidazo[1,2-a]pyridin-2-yl)methyl)carbamate

A mixture of tert-butyl ((6-cyclopropyl-8-(3-methyl-2,4-dioxoimidazolidin-1-yl)imidazo[1,2-a]pyridin-2-yl)methyl)carbamate (200 mg, 0.5 mmol), Selectfluor (354 mg, 1.0 mmol) and DMAP (61 mg, 0.5 mmol) in DCM/MeOH (3.0 mL/1.0 mL) was stirred at room temperature for 16 h. The reaction was concentrated in vacuo to give the crude product, which was purified by silica gel chromatography (eluent: DCM/MeOH=30/1˜10/1) to give tert-butyl ((6-cyclopropyl-3-fluoro-8-(3-methyl-2,4-dioxoimidazolidin-1-yl)imidazo[1,2-a]pyridin-2-yl)methyl)carbamate as a white solid (120 mg, 58%). ESI-MS [M+H]⁺: 418.2.

### Synthesis of 1-(2-(aminomethyl)-6-cyclopropyl-3-fluoroimidazo[1,2-a]pyridin-8-yl)-3-methylimidazolidine-2,4-dione

A mixture of tert-butyl ((6-cyclopropyl-3-fluoro-8-(3-methyl-2,4-dioxoimidazolidin-1-yl)imidazo[1,2-a]pyridin-2-yl)methyl)carbamate (84 mg, 0.2 mmol) in HCl (4 M in dioxane, 5 mL) was stirred at room temperature for 1 h. The reaction mixture was concentrated in vacuo to give 1-(2-(aminomethyl)-6-cyclopropyl-3-fluoroimidazo[1,2-a]pyridin-8-yl)-3-methylimidazolidine-2,4-dione as the hydrochloric acid salt (80 mg, crude) as a white solid, which was used directly in the next step. ESI-MS [M+H]⁺: 318.2.

### Synthesis of 1-(2-(aminomethyl)-6-cyclopropylimidazo[1,2-a]pyridin-8-yl)-3-(4-methoxybenzyl)imidazolidine-2,4-dione

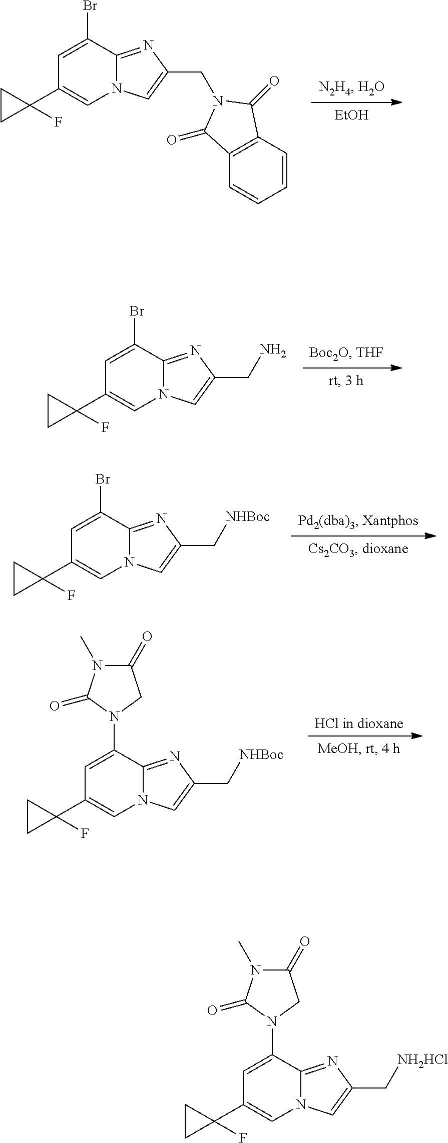

### Synthesis of (8-bromo-6-(1-fluorocyclopropyl)imidazo[1,2-a]pyridin-2-yl)methanamine

To a solution of 2-((8-bromo-6-(1-fluorocyclopropyl)imidazo[1,2-a]pyridin-2-yl)methyl)isoindoline-1,3-dione (200 mg, 0.48 mmol) in EtOH (10 mL) was added N₂H₄⁻H₂O (1 mL). The reaction mixture was stirred at 65° C. for 2 h and then allowed to reach room temperature. The solution was filtered and the filtrate was concentrated in vacuo to give (8-bromo-6-(1-fluorocyclopropyl)imidazo[1,2-a]pyridin-2-yl)methanamine (100 mg, crude) as a white solid, which was used in the next step without further purification. ESI-MS [M+H]⁺: 284.1.

### Synthesis of tert-butyl ((8-bromo-6-(1-fluorocyclopropyl)imidazo[1,2-a]pyridin-2-yl)methyl)carbamate

To a solution of (8-bromo-6-(1-fluorocyclopropyl)imidazo[1,2-a]pyridin-2-yl)methanamine (100 mg, crude) in dry THF (10 mL) was added Boc₂O (152 mg, 0.70 mmol) and trimethylamine (106 mg, 1.05 mmol). After stirring for 3 h at room temperature, water (10 mL) was added and the mixture was extracted with EtOAc (3×10 mL). The combined organic layers were washed with brine (20 mL), dried over anhydrous Na₂SO₄ then concentrated to give the crude, which was purified by preparative TLC (eluent: PE/EtOAc=3/1) to give tert-butyl ((8-bromo-6-(1-fluorocyclopropyl)imidazo[1,2-a]pyridin-2-yl)methyl)carbamate (120 mg, 65% in 2 steps) as a white solid. ESI-MS [M+H]⁺: 384.2.

### Synthesis of tert-butyl ((6-(1-fluorocyclopropyl)-8-(3-methyl-2,4-dioxoimidazolidin-1-yl)imidazo[1,2-a]pyridin-2-yl)methyl)carbamate

To a mixture of tert-butyl ((8-bromo-6-(1-fluorocyclopropyl)imidazo[1,2-a]pyridin-2-yl)methyl)carbamate (120 mg, 0.31 mmol), 3-methylimidazolidine-2,4-dione (71 mg, 0.62 mmol), and Cs₂CO₃ (303 mg, 0.93 mmol) in dioxane (10 mL) was added Pd₂(dba)₃ (28 mg, 0.031 mmol) and Xantphos (18 mg, 0.031 mmol). The reaction mixture was stirred at 90° C. for 16 h under N₂ and cooled to room temperature. The reaction mixture was filtered through celite and the filter cake was washed with DCM/MeOH (10/1, 20 mL). The filtrate was concentrated in vacuo to give the crude, which was purified by silica gel chromatography (eluent: DCM/MeOH=50/1˜20/1) to give tert-butyl ((6-(1-fluorocyclopropyl)-8-(3-methyl-2,4-dioxoimidazolidin-1-yl)imidazo[1,2-a]pyridin-2-yl)methyl)carbamate (100 mg, 77%) as a yellow solid. ESI-MS [M+H]+: 418.2.

### Synthesis of 1-(2-(aminomethyl)-6-(1-fluorocyclopropyl)imidazo[1,2-a]pyridin-8-yl)-3-methylimidazolidine-2,4-dione

To a solution of tert-butyl ((6-(1-fluorocyclopropyl)-8-(3-methyl-2,4-dioxoimidazolidin-1-yl)imidazo[1,2-a]pyridin-2-yl)methyl)carbamate (100 mg, 0.24 mmol) in MeOH (5 mL) was added HCl (4M solution in dioxane, 5 mL). The resulting mixture was stirred at room temperature for 4 h and then concentrated in vacuo to give 1-(2-(aminomethyl)-6-(1-fluorocyclopropyl)imidazo[1,2-a]pyridin-8-yl)-3-methylimidazolidine-2,4-dione as the hydrochloric acid salt (80 mg, 85%) as a yellow oil which was used in next step without purification. ESI-MS [M+H]+: 318.1.

### Synthesis of 3-(2-(benzyloxy)ethyl)imidazolidine-2,4-dione

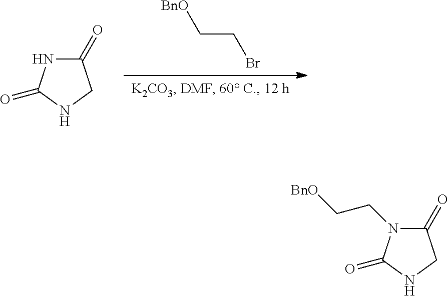

A solution of imidazolidine-2,4-dione (500 mg, 5 mmol), ((2-bromoethoxy)methyl)benzene (1.18 g, 5.5 mmol) and K₂CO₃ (1.38 g, 10 mmol) in DMF (10 mL) was stirred at 60° C. for 12 h. The reaction mixture was cooled to room temperature, poured into water (50 mL) and extracted with EtOAc (2×50 ml). The combined organic layers were washed with brine (100 mL), dried over Na₂SO₄ then concentrated in vacuo to give the crude product, which was purified by silica gel chromatography (eluent: EtOAc/PE=1/1) to give 3-(2-(benzyloxy)ethyl)imidazolidine-2,4-dione (720 mg, 62%) as a white solid. ESI-MS [M+H]⁺: 235.1.

### Synthesis of 3-(oxetan-3-yl)imidazolidine-2,4-dione

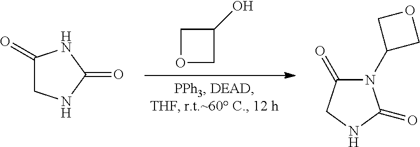

To a solution of imidazolidine-2,4-dione (3 g, 30 mmol), oxetan-3-ol (6.66 g, 90 mmol) and PPh₃ (15.78 g, 60 mmol) in THF (50 mL) was added DEAD (10.44 g, 60 mmol). The resulting mixture was stirred at 60° C. for 12 h, then water (100 mL) was added. The mixture was extracted with EtOAc (3×80 mL). The combined organic layers were washed with brine (80 mL), dried over Na₂SO₄ and concentrated in vacuo to give the crude product, which was purified with silica gel chromatography (eluent: DCM/MeOH=15/1) to give 3-(oxetan-3-yl)imidazolidine-2,4-dione (1 g, 21%) as a yellow solid. ESI-MS [M+H]⁺:157.2.

### Synthesis of ethyl 2-(2,5-dioxoimidazolidin-1-yl)acetate

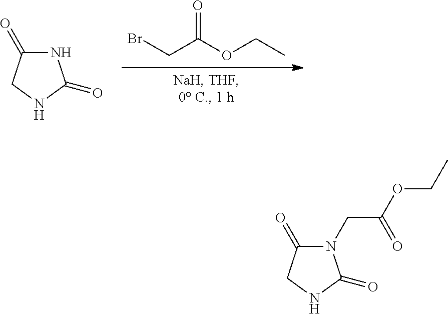

To a solution of imidazolidine-2,4-dione (2.0 g, 20.0 mmol) in dry THF (50 mL) was added NaH (1.2 g, 30 mmol) at 0° C. The mixture was stirred for 0.5 h, then ethyl 2-bromoacetate (3.3 g, 20.0 mmol) was added. The reaction was stirred at room temperature for another 1 h, then quenched with sat·NH₄Cl solution (50 mL) and extracted with EtOAc (2×50 mL). The combined organic layers were washed with brine (50 mL), dried over anhydrous Na₂SO₄ and concentrated inn vacuo to give the crude product, which was purified by silica gel chromatography (eluent: DCM/MeOH=10/1) to give ethyl 2-(2,5-dioxoimidazolidin-1-yl)acetate (500 mg, 13%) as a brown oil. ESI-MS [M+H]⁺: 187.2.

### Synthesis of 3-cyclopropylimidazolidine-2,4-dione

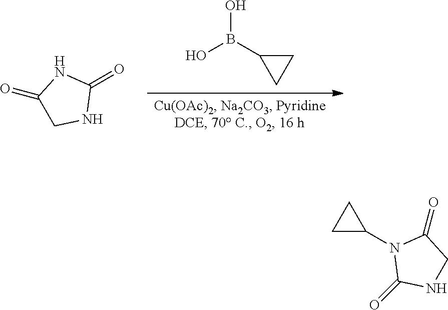 [SMILES: O=C1CNC(=O)N1 ; OB(O)C1CC1 ; "Cu(OAc)2, Na2CO3, PyridineDCE, 70° C., O2, 16 h" ; O=C1CNC(=O)N1C1CC1]

To a mixture of imidazolidine-2,4-dione (1 g, 10 mmol), cyclopropylboronic acid (903 mg, 10.5 mmol), Cu(OAc)₂ (3.73 g, 20.5 mmol), and Na₂CO₃ (2.17 g, 20.5 mmol) in DCE (100 mL) was added pyridine (3.95 g, 50 mmol). After stirring at 70° C. for 16 h under 02, the reaction mixture was filtered through celite and the filter cake was washed with DCM/MeOH (10/1, 100 mL). The filtrate was concentrated in vacuo to give the crude product, which was purified by silica gel chromatography (eluent: MeOH/DCM=1/100˜1/5) to afford 3-cyclopropylimidazolidine-2,4-dione (190 mg, 14%) as white solid. ESI-MS [M+H]+: 141.2.

### Synthesis of 3-(2,2,2-trifluoroethyl)imidazolidine-2,4-dione

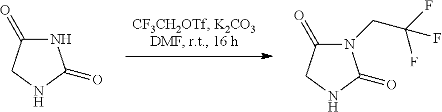 [SMILES: O=C1CNC(=O)N1 ; "CF3CH2OTf, K2CO3DMF, r.t., 16 h" ; O=C1CNC(=O)N1CC(F)(F)F]

To a mixture of imidazolidine-2,4-dione (1 g, 10 mmol) in DMF (30 mL) was added K₂CO₃ (2.76 g, 20 mmol) and CF₃CH₂OTf (2.55 g, 11 mmol). The reaction was stirred at room temperature under N₂ for 16 h. The reaction was poured into water (50 ml) and extracted with EtOAc (3×50 ml). The combined organic layers were washed with brine (50 mL), dried over anhydrous Na₂SO₄ and concentrated in vacuo to give the crude, which was purified by silica gel chromatography (eluent: DCM/MeOH=40/1) to give the 3-(2,2,2-trifluoroethyl)imidazolidine-2,4-dione (1 g, 55%) as yellow solid. ESI-MS [M+H]⁺: 183.0.

### Synthesis of 3-(4-methoxybenzyl)imidazolidine-2,4-dione

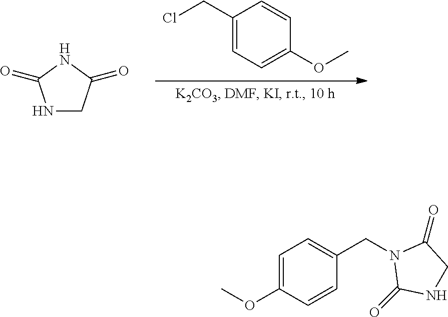

A mixture of imidazolidine-2,4-dione (1.0 g, 10 mmol), 1-(chloromethyl)-4-methoxybenzene (1.56 g, 10 mmol), K₂CO₃ (2.07 g, 15 mmol) and KI (166 mg, 1 mmol) in DMF (40 ml) was stirred at rt for 10 h. The reaction was quenched with water (50 mL) and extracted with EtOAc (3×50 mL). The combined organic layers were washed with brine (50 mL), dried over anhydrous Na₂SO₄ and concentrated in vacuo to give the crude product, which was purified by silica gel chromatography (eluent: PE/EtOAc=1/1) to give 3-(4-methoxybenzyl)imidazolidine-2,4-dione (1.0 g, 45%) as a yellow solid. ESI-MS: [M+H]+, 221.2.

### Synthesis of 3-methyldihydropyrimidine-2,4(1H,3H)-dione

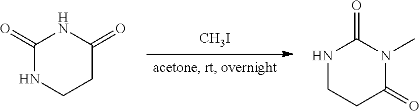

To a mixture of dihydropyrimidine-2,4(1H,3H)-dione (500 mg, 4.38 mmol) in acetone (5 mL) was added K₂CO₃ (1.09 g, 7.89 mmol) and iodomethane (1.12 g, 7.89 mmol) successively. After stirring at room temperature overnight, the resulting mixture was filtered through celite and the filter cake was washed with DCM/MeOH (10/1, 15 mL). The filtrate was concentrated in vacuo to give the crude, which was purified by was purified by silica gel chromatography (eluent: MeOH/DCM=1/100˜20/1) to give 3-methyldihydropyrimidine-2,4(1H,3H)-dione (44 mg, 8%) as white solid. ESI-MS [M+H]+: 129.1.

### Synthesis of (6-cyclopropyl-8-(4-methylpiperazin-1-yl)imidazo[1,2-a]pyridin-2-yl)methanamine

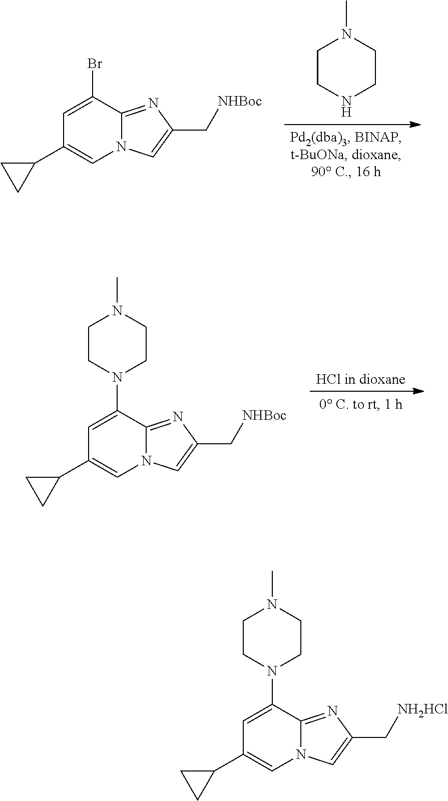

### Synthesis of tert-butyl ((6-cyclopropyl-8-(4-methylpiperazin-1-yl)imidazo[1,2-a]pyridin-2-yl)methyl)carbamate

To a mixture of tert-butyl ((7-bromo-5-cyclopropylpyrazolo[1,5-a]pyridin-2-yl)methyl)carbamate (2 g, 5.5 mmol), 1-methylpiperazine (1.64 g, 16.4 mmol) and t-BuONa (1.58 g, 16.4 mmol) in 1,4-dioxane (50 mL) was added Pd₂(dba)₃ (504 mg, 0.55 mmol) and Xantphos (636 mg, 1.1 mmol). After stirring at 90° C. for 16 h under N₂, the reaction mixture was filtered through celite and the filter cake was washed with DCM/MeOH (10/1, 50 mL). The filtrate was concentrated in vacuo to give the crude product, which was purified by silica gel chromatography (eluent: DCM/MeOH=10/1) to give tert-butyl ((6-cyclopropyl-8-(4-methylpiperazin-1-yl)imidazo[1,2-a]pyridin-2-yl)methyl)carbamate (900 mg, 431) as yellow solid. ESI-MS [M+H]⁺: 386.2.

### Synthesis of (6-cyclopropyl-8-(4-methylpiperazin-1-yl)imidazo[1,2-a]pyridin-2-yl)methanamine

To a solution of tert-butyl ((6-cyclopropyl-8-(4-methylpiperazin-1-yl)imidazo[1,2-a]pyridin-2-yl)methyl)carbamate (900 mg, 2.3 mmol) in dioxane (10 mL) was added HCl (4M solution in dioxane, 10 mL). The resulting mixture was stirred at room temperature for 1 h and then concentrated in vacuo to give (6-cyclopropyl-8-(4-methylpiperazin-1-yl)imidazo[1,2-a]pyridin-2-yl)methanamine as the hydrochloric acid salt (920 mg, crude) as a yellow solid, which was used in the next step without further purification. ESI-MS [M+H]⁺: 286.2.

Using a similar procedure to that used for (6-cyclopropyl-8-(4-methylpiperazin-1-yl)imidazo[1,2-a]pyridin-2-yl)methanamine, the compounds in Table A10 were prepared from tert-butyl ((8-bromo-6-cyclopropylimidazo[1,2-a]pyridin-2-yl)methyl)carbamate and an appropriate coupling partner, followed by treatment HCl in dioxane.

TABLE A10

| Structure | Coupling Partner | Analytical Data |
|---|---|---|
| 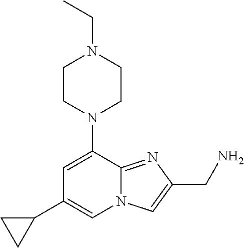 [SMILES: CCN1CCN(c2cc(C3CC3)cn3cc(CN)nc23)CC1] | 1-ethylpiperazine | ESI-MS [M + H]+: 300.1 |
| (6-cyclopropyl-8-(4-ethylpiperazin-1- | | |
| yl)imidazo[1,2-a]pyridin-2- | | |
| yl)methanamine hydrochloride | | |
| 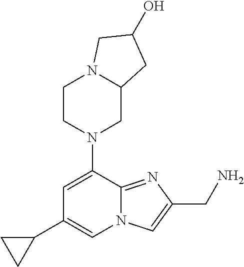 [SMILES: NCc1cn2cc(C3CC3)cc(N3CCN4CC(O)CC4C3)c2n1] | octahydropyrrolo[1,2-a]pyrazin- 7-ol | ESI-MS [M + H]+: 327.2 |
| 2-(2-(aminomethyl)-6- | | |
| cyclopropylimidazo[1,2-a]pyridin-8- | | |
| yl)octahydropyrrolo[1,2-a]pyrazin-7-ol | | |
| hydrochloride | | |
| 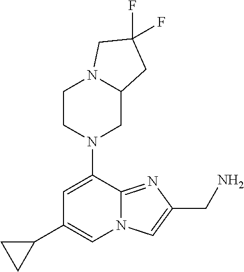 [SMILES: NCc1cn2cc(C3CC3)cc(N3CCN4CC(F)(F)CC4C3)c2n1] | 7,7-difluorooctahydro-pyrrolo [1,2-a]pyrazine | ESI-MS [M + H]+: 163.2 |
| (6-cyclopropyl-8-(7,7- | | |
| difluorohexahydropyrrolo[1,2-a]pyrazin- | | |
| 2(1H)-yl)imidazo[1,2-a]pyridin-2- | | |
| yl)methanamine hydrochloride | | |
| 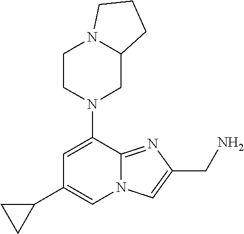 [SMILES: NCc1cn2cc(C3CC3)cc(N3CCN4CCCC4C3)c2n1] | octahydropyrrolo[1,2-a]pyrazine | ESI-MS [M + H]⁺: 312.2 |
| (6-cyclopropyl-8-(hexahydropyrrolo[1,2- | | |
| a]pyrazin-2(1H)-yl)imidazo[1,2- | | |
| a]pyridin-2-yl)methanamine | | |
| hydrochloride | | |

### Synthesis of 1-(2-(aminomethyl)-6-cyclopropylimidazo[1,2-a]pyridin-8-yl)-3-(2-(2-methoxyethoxy)ethyl)imidazolidine-2,4-dione

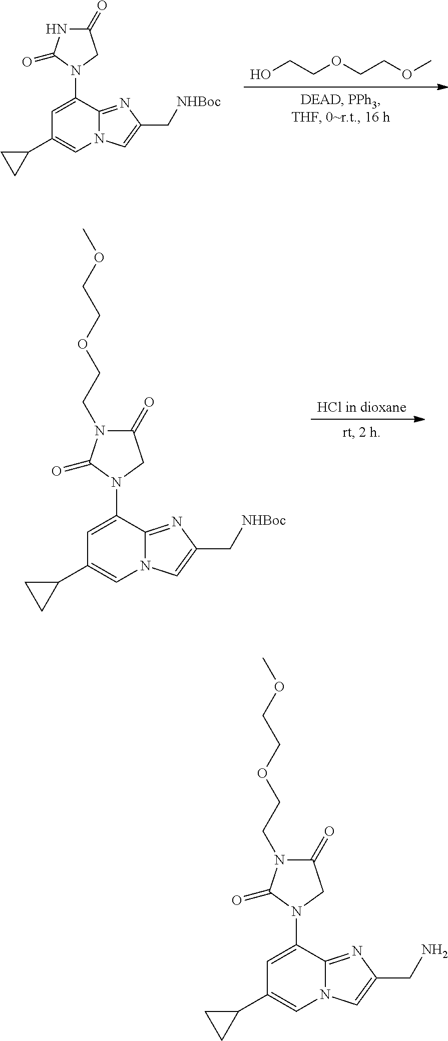

### Synthesis of tert-butyl ((6-cyclopropyl-8-(3-(2-(2-methoxyethoxy)ethyl)-2,4-dioxoimidazolidin-1-yl)imidazo[1,2-a]pyridin-2-yl)methyl)carbamate

To a mixture of tert-butyl ((6-cyclopropyl-8-(2,4-dioxoimidazolidin-1-yl)imidazo[1,2-a]pyridin-2-yl)methyl)carbamate (145 mg, 0.38 mmol) and PPh₃ (199 mg, 0.76 mol) in THF (10 mL) was added 2-(2-methoxyethoxy)ethan-1-ol (91 mg, 0.76 mol) at 0° C. Diethylazodicarboxylate (198 mg, 1.14 mmol) was added dropwise to the reaction and the mixture was stirred at room temperature under N₂ for 16 h. The reaction was concentrated in vacuo to give the crude, which was purified by preparative TLC (eluent: DCM/MeOH: 15/1) to give tert-butyl ((6-cyclopropyl-8-(3-(2-(2-methoxyethoxy)ethyl)-2,4-dioxoimidazolidin-1-yl)imidazo[1,2-a]pyridin-2-yl)methyl)carbamate (50 mg, 27%) as yellow oil. ESI-MS [M+H]⁺: 488.2.

### Synthesis of 1-(2-(aminomethyl)-6-cyclopropylimidazo[1,2-a]pyridin-8-yl)-3-(2-(2-methoxyethoxy)ethyl)imidazolidine-2,4-dione

To a solution of tert-butyl ((6-cyclopropyl-8-(3-(2-(2-methoxyethoxy)ethyl)-2,4-dioxoimidazolidin-1-yl)imidazo[1,2-a]pyridin-2-yl)methyl)carbamate (50 mg, 0.1 mol) in dioxane (2 mL) was added HCl (4M solution in dioxane, 2 mL). After stirring at room temperature for 2 h, the mixture was basified (pH 8) with sat·aqueous NaHCO₃ solution then extracted with DCM (3×20 mL). The combined organic layers were washed with brine (30 mL), dried over anhydrous Na₂SO₄ and concentrated in vacuo to give the crude product, which was purified with preparative TLC (eluent: DCM/MeOH=10/1) to afford 1-(2-(aminomethyl)-6-cyclopropylimidazo[1,2-a]pyridin-8-yl)-3-(2-(2-methoxyethoxy)ethyl)imidazolidine-2,4-dione (38 mg, 98%) as yellow solid. ESI-MS [M+H]⁺: 388.2.

### Synthesis of 1-((2-(aminomethyl)-6-cyclopropylimidazo[1,2-a]pyridin-8-yl)methyl)-3-methyl

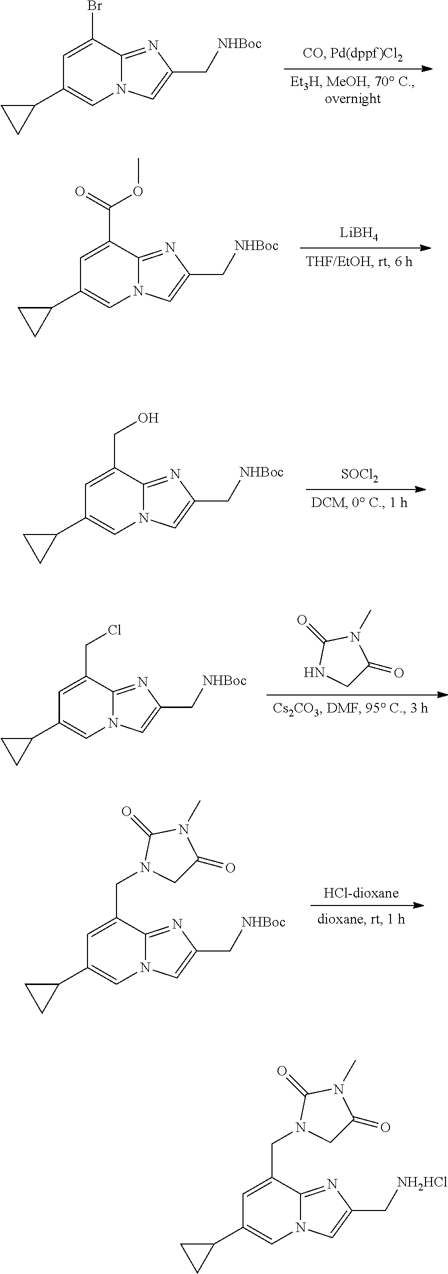

### Synthesis of methyl 2-(((tert-butoxycarbonyl)amino)methyl)-6-cyclopropylimidazo[1,2-a]pyridine-8-carboxylate

To a mixture of tert-butyl ((8-bromo-6-cyclopropylimidazo[1,2-a]pyridin-2-yl)methyl)carbamate (500 mg, 1.37 mmol) and Et₃N (693 mg, 6.85 mmol) in MeOH (15 ml) was added Pd(dppf)Cl₂ (102 mg, 0.14 mmol). The mixture was stirred at 70° C. under CO overnight and cooled to room temperature. The reaction mixture was filtered and the filtrate was concentrated in vacuo to give the crude, which was purified by silica gel chromatography (eluent: PE/EtOAc=1/2) to give methyl 2-(((tert-butoxycarbonyl)amino)methyl)-6-cyclopropylimidazo[1,2-a]pyridine-8-carboxylate (410 mg, 87%) as an orange solid. ESI-MS [M+H]⁺: 346.2.

### Synthesis of tert-butyl ((6-cyclopropyl-8-(hydroxymethyl)imidazo[1,2-a]pyridin-2-yl)methyl)carbamate

To a solution of methyl 2-(((tert-butoxycarbonyl)amino)methyl)-6-cyclopropylimidazo[1,2-a] pyridine-8-carboxylat (420 mg, 1.22 mmol) in THF/EtOH (5/1, 24 mL) was added LiBH₄ (80 mg, 3.67 mmol). The reaction mixture was stirred at room temperature for 6 h, then quenched with saturated NH₄Cl (10 mL) and extracted with EtOAc (3×20 mL). The combined organic layers were washed with brine (20 mL), dried over anhydrous Na₂SO₄, and concentrated in vacuo to give tert-butyl ((6-cyclopropyl-8-(hydroxymethyl)imidazo[1,2-a]pyridin-2-yl)methyl)carbamate (397 mg, crude) as a yellow solid. ESI-MS [M+H]⁺: 318.2.

### Synthesis of tert-butyl ((8-(chloromethyl)-6-cyclopropylimidazo[1,2-a]pyridin-2-yl)methyl)carbamate

To a solution of tert-butyl ((6-cyclopropyl-8-(hydroxymethyl)imidazo[1,2-a]pyridin-2-yl)methyl) carbamate (350 mg, 1.10 mmol) in DCM (3.5 mL) was added SOCl₂ (131 mg, 1.10 mmol) at 0° C. for 1 h. The resulting mixture was concentrated in vacuo to give tert-butyl ((8-(chloromethyl)-6-cyclopropylimidazo[1,2-a]pyridin-2-yl)methyl)carbamate (413 mg, crude) as a yellow solid which was used in next step without purification. ESI-MS [M+H]⁺: 336.2.

### Synthesis of tert-butyl ((6-cyclopropyl-8-((3-methyl-2,4-dioxoimidazolidin-1-yl)methyl)imidazo[1,2-a]pyridin-2-yl)methyl)carbamate

A mixture of tert-butyl ((8-(chloromethyl)-6-cyclopropylimidazo[1,2-a]pyridin-2-yl)methyl) carbamate (413 mg, crude), 3-methylimidazolidine-2,4-dione (146 mg, 1.28 mmol), and Cs₂CO₃ (1.251 g, 3.84 mmol) in DMF (10.0 ml) was stirred in a sealed tube at 95° C. for 3 h. The resulting mixture was cooled to room temperature and concentrated in vacuo to give the crude, which was purified by column chromatography (eluent: DCM/MeOH=10/1) to give tert-butyl ((6-cyclopropyl-8-((3-methyl-2,4-dioxoimidazolidin-1-yl)methyl)imidazo[1,2-a]pyridin-2-yl)methyl)carbamate (82 mg, 18%) as a yellow solid. ESI-MS [M+H]⁺: 414.2.

### Synthesis of 1-((2-(aminomethyl)-6-cyclopropylimidazo[1,2-a]pyridin-8-yl)methyl)-3-methyl imidazolidine-2,4-dione hydrochloride

To a solution of tert-butyl ((6-cyclopropyl-8-((3-methyl-2,4-dioxoimidazolidin-1-yl)methyl)imidazo [1,2-a]pyridin-2-yl)methyl)carbamate (82 mg, 0.20 mmol) in dioxane (2 ml) was added HCl (4 M solution in dioxane, 2 mL, 0.8 mmol) at room temperature. After stirring the resulting mixture for 1 h, the mixture was concentrated in vacuo to give 1-((2-(aminomethyl)-6-cyclopropylimidazo[1,2-a]pyridin-8-yl)methyl)-3-methylimidazolidine-2,4-dione hydrochloride (83 mg, crude) as a yellow solid which was used in the next step without purification. ESI-MS [M+H]⁺: 350.1.

### Synthesis of 1-(6-cyclopropyl-2-((methylamino)methyl)imidazo[1,2-a]pyridin-8-yl)-3-methylimidazolidine-2,4-dione

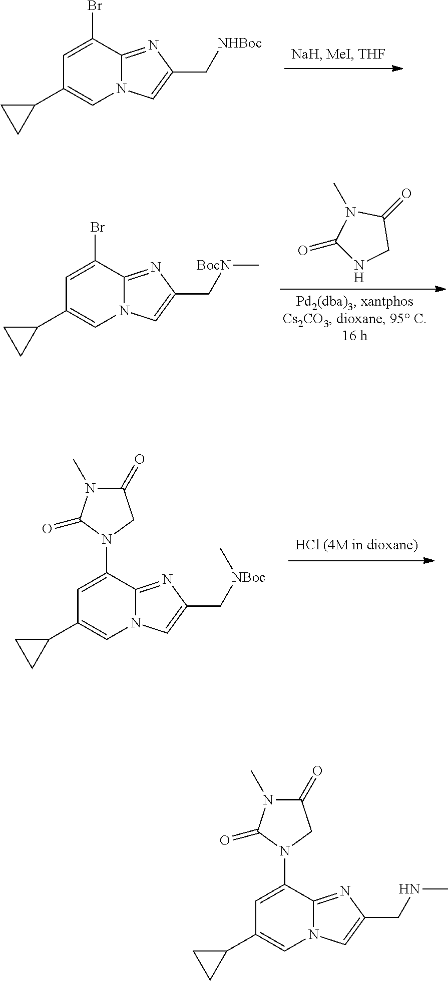

### Synthesis of tert-butyl ((8-bromo-6-cyclopropylimidazo[1,2-a]pyridin-2-yl)methyl)(methyl)carbamate

To a solution of tert-butyl ((8-bromo-6-cyclopropylimidazo[1,2-a]pyridin-2-yl)methyl)carbamate (200 mg, 0.55 mmol) in THF (6 mL) was added NaH (32.9 mg, 60% in oil, 0.82 mmol) at 0° C. The reaction mixture was stirred at 0° C. for 0.5 h under N₂ and then a solution of MeI (116.4 mg, 0.82 mmol) in THF (1 mL) was added. After stirring the mixture at room temperature for 2 h, the mixture was quenched with sat·aq·NH₄Cl solution (30 mL) and extracted with EtOAc (3×30 mL). The combined organic layers were washed with brine (20 mL), dried over Na₂SO₄ and concentrated. The crude residue was purified by preparative TLC (eluent: PE/EtOAc=2/1) to give tert-butyl ((8-bromo-6-cyclopropylimidazo[1,2-a]pyridin-2-yl)methyl)(methyl)carbamate (170 mg, 82%) as a white solid. ESI-MS [M+H]+: 380.2.

### Synthesis of (tert-butyl ((6-cyclopropyl-8-(3-methyl-2,4-dioxoimidazolidin-1-yl)imidazo[1,2-a]pyridin-2-yl)methyl)(methyl)carbamate

To a mixture of tert-butyl ((8-bromo-6-cyclopropylimidazo[1,2-a]pyridin-2-yl)methyl)(methyl)carbamate (170 mg, 0.45 mmol), 3-methylimidazolidine-2,4-dione (154 mg, 1.35 mmol), and Cs₂CO₃ (440 mg, 1.35 mmol) in dioxane (5 mL) was added Pd₂(dba)₃ (41 mg, 0.045 mmol) and XantPhos (52 mg, 0.09 mmol). The reaction mixture was stirred at 95° C. for 16 h under N₂. The reaction mixture was cooled to room temperature, filtered through Celite® and the filter cake was washed with DCM/MeOH (10/1, 20 mL). The filtrate was concentrated to give the crude residue, which was purified by preparative TLC (eluent: DCM/MeOH=20/1) to give tert-butyl ((6-cyclopropyl-8-(3-methyl-2,4-dioxoimidazolidin-1-yl)imidazo[1,2-a]pyridin-2-yl)methyl)(methyl)carbamate (80 mg, 43%) as a white solid. ESI-MS [M+H]+: 414.2.

### Synthesis of 1-(6-cyclopropyl-2-((methylamino)methyl)imidazo[1,2-a]pyridin-8-yl)imidazolidine-2,4-dione

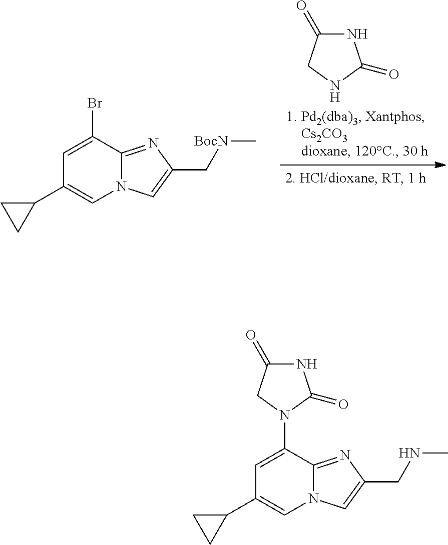

### Synthesis of tert-butyl ((6-cyclopropyl-8-(2,4-dioxoimidazolidin-1-yl)imidazo[1,2-a]pyridin-2-yl)methyl)(methyl)carbamate

To a solution of tert-butyl ((8-bromo-6-cyclopropylimidazo[1,2-a]pyridin-2-yl)methyl)(methyl)carbamate (1.14 g, 3 mmol) in dioxane (30 mL) were added imidazolidine-2,4-dione (1.2 g, 12 mmol), Cs₂CO₃ (2.93 g, 9 mmol), Xantphos (520 mg, 0.9 mmol) and Pd₂(dba)₃ (411 mg, 0.45 mmol) under N₂. The resulting reaction was then stirred at 120° C. for 16 h and cooled to room temperature. The reaction mixture was poured into water (50 mL) and extracted with EtOAc (3×50 ml). The combined organics were washed with brine (50 mL), dried over Na₂SO₄ then concentrated to give the crude product, which was purified by silica gel chromatography (eluent: EtOAc/PE=4/1) to give tert-butyl ((6-cyclopropyl-8-(2,4-dioxoimidazolidin-1-yl)imidazo[1,2-a]pyridin-2-yl)methyl)(methyl)carbamate (650 mg, 54%) as a yellow solid. ESI-MS [M+H]⁺: 400.2.

### Synthesis of 1-(6-cyclopropyl-2-((methylamino)methyl)imidazo[1,2-a]pyridin-8-yl)imidazolidine-2,4-dione

To a solution of tert-butyl ((6-cyclopropyl-8-(2,4-dioxoimidazolidin-1-yl)imidazo[1,2-a]pyridin-2-yl)methyl)(methyl)carbamate (650 mg, 1.63 mmol) in MeOH (10 mL) was added HCl/dioxane (10 ml, 6 mol/L in dioxane). The resulting reaction was stirred at room temperature for 1 h and then concentrated in vacuo to give 1-(6-cyclopropyl-2-((methylamino)methyl)imidazo[1,2-a]pyridin-8-yl)imidazolidine-2,4-dione (650 mg, crude) as a yellow solid which was used in next step without purification. ESI-MS [M+H]⁺: 300.2.

Using a similar procedure to that used for 1-(6-cyclopropyl-2-((methylamino)methyl)imidazo[1,2-a]pyridin-8-yl)imidazolidine-2,4-dione, the compounds in Table A11 were prepared from tert-butyl ((6-cyclopropyl-8-(2,4-dioxoimidazolidin-1-yl)imidazo[1,2-a]pyridin-2-yl)methyl)(methyl)carbamate and an appropriate coupling partner, followed by treatment with HCl in dioxane.

TABLE A11

| Structure | Coupling Partner | Analytical Data |
|---|---|---|
| 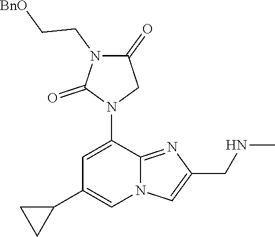 [SMILES: CNCc1cn2cc(C3CC3)cc(N3CC(=O)N(CCOCc4ccccc4)C3=O)c2n1] | 3-(2-(benzyloxy) ethyl)imidazolidine-2,4- dione | ESI-MS [M + H]⁺: 434.2 |
| 13-(2-(benzyloxy)ethyl)-1-(6- | | |
| cyclopropyl-2-((methylamino) | | |
| methyl)imidazo[1,2-a]pyridin-8- | | |
| yl)imidazolidine-2,4-dione hydrochloride | | |

### Synthesis of 1-(6-cyclopropyl-2-((ethylamino)methyl)imidazo[1,2-a]pyridin-8-yl)-3-methylimidazolidine-2,4-dione

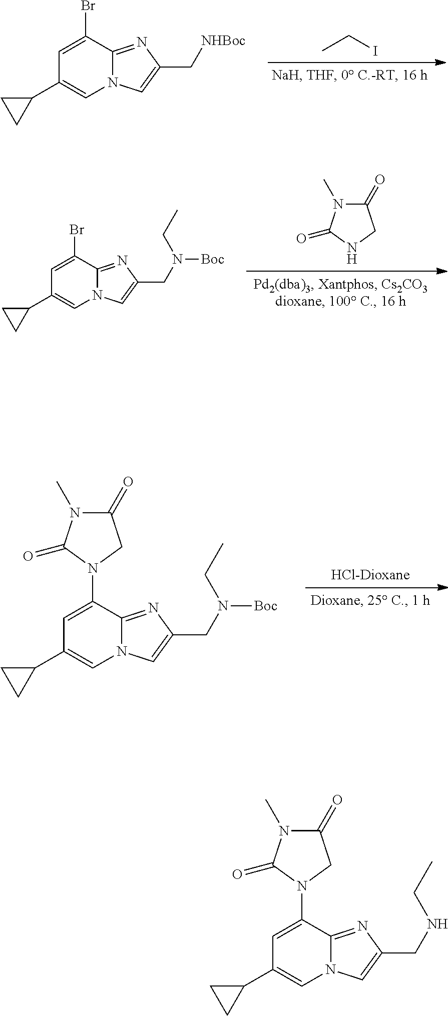

Using a similar procedure to that for tert-butyl ((6-cyclopropyl-8-(3-methyl-2,4-dioxoimidazolidin-1-yl)imidazo[1,2-a]pyridin-2-yl)methyl)(methyl)carbamate, 1-(6-cyclopropyl-2-((methylamino)methyl)imidazo[1,2-a]pyridin-8-yl)-3-methylimidazolidine-2,4-dione (60 mg) was synthesised from tert-butyl ((8-bromo-6-cyclopropylimidazo[1,2-a]pyridin-2-yl)methyl)carbamate and ethyl iodide. ESI-MS [M+H]+: 328.2.

### Synthesis of benzyl ((6-cyclopropyl-8-(3-methyl-2,4-dioxoimidazolidin-1-yl)imidazo[1,2-a]pyridin-2-yl)methyl)glycinate

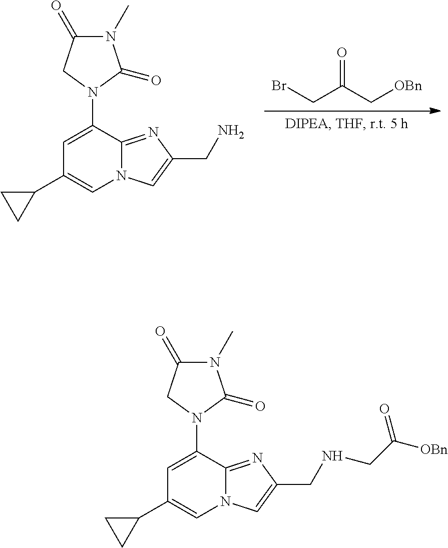

To a solution of 1-(2-(aminomethyl)-6-cyclopropylimidazo[1,2-a]pyridin-8-yl)-3-methylimidazolidine-2,4-dione (299 mg, 1.0 mmol) and DIPEA (645 mg, 5.0 mmol) in THF (20 mL) was added 1-(benzyloxy)-3-bromopropan-2-one (242 mg, 1.0 mmol) at 0° C. The mixture was stirred at room temperature for 5h, quenched with water (30 mL) and extracted with EtOAc (3×10 mL). The combined organic layers were washed with brine (20 mL), dried over anhydrous Na₂SO₄ and concentrated in vacuo to give the crude product, which was purified with preparative TLC (eluent: DCM/MeOH=10/1) to give the product benzyl ((6-cyclopropyl-8-(3-methyl-2,4-dioxoimidazolidin-1-yl)imidazo[1,2-a]pyridin-2-yl)methyl)glycinate as a yellow oil (190 mg, 42%). ESI-MS [M+H]+: 448.1.

### Synthesis of 1-(6-cyclopropyl-2-((methylamino)methyl)imidazo[1,2-a]pyridin-8-yl)-3-(2-hydroxy-2-methylpropyl)imidazolidine-2,4-dione

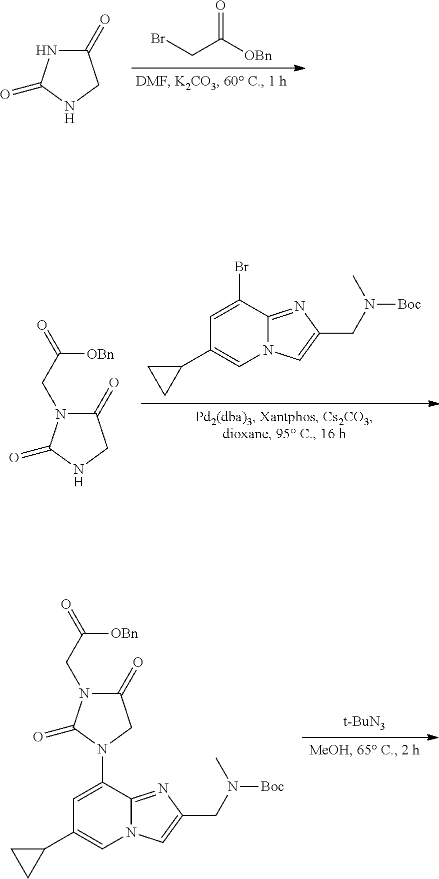

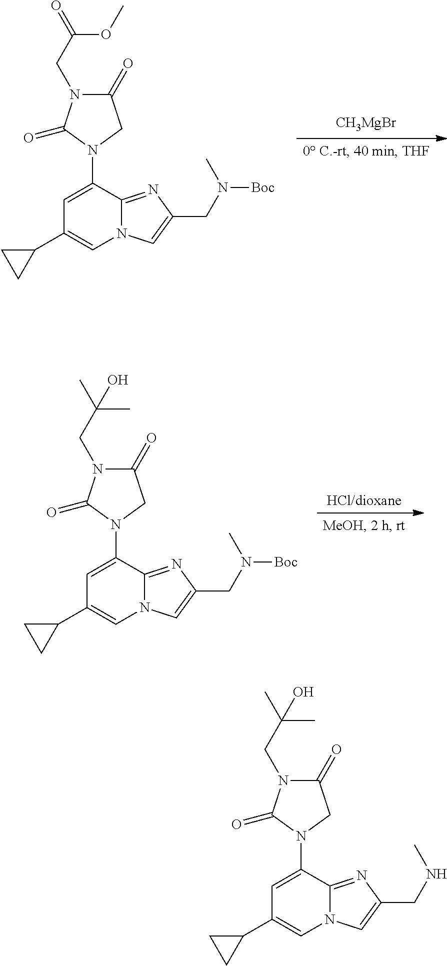

### Synthesis of benzyl 2-(2,5-dioxoimidazolidin-1-yl)acetate

To a solution of imidazolidine-2,4-dione (2 g, 20.0 mmol) in DMF (20 mL) was added benzyl 2-bromoacetate (3.2 g, 14.0 mmol) and K₂CO₃ (5.5 g, 40.0 mmol). After stirring the mixture was stirred at 60° C. for 1 h, quenched with water (50 mL) and extracted with EtOAc (3×30 mL). The combined organics were washed with brine (50 mL), dried over Na₂SO₄ and concentrated in vacuo to give benzyl 2-(2,5-dioxoimidazolidin-1-yl)acetate (2.5 g, 50.4%) as a white solid. ESI-MS [M+Na]⁺: 271.1.

### Synthesis of benzyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate

A mixture of benzyl 2-(2,5-dioxoimidazolidin-1-yl)acetate (2.35 g, 9.5 mmol), tert-butyl ((8-bromo-6-cyclopropylimidazo[1,2-a]pyridin-2-yl)methyl)(methyl)carbamate (900 mg, 2.4 mmol), Pd₂(dba)₃ (433 mg, 0.2 mmol), Xantphos (548 mg, 0.95 mmol) and Cs₂CO₃ (2.3 g, 7.0 mmol) in dioxane (20 mL) was stirred at 95° C. for 16 h under N₂. The mixture was warmed to room temperature, quenched with water (50 mL) and extracted with DCM (3×50 ml). The combined organics were washed with brine (50 mL), dried over Na₂SO₄ and concentrated. The residue was purified by silica gel chromatography (eluant: PE:EtAOc=1:1) to give benzyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (600 mg, 46%) as a yellow solid. ESI-MS [M+H]⁺: 548.2.

### Synthesis of methyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate

To a solution of benzyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (600 mg, 1.09 mmol) in MeOH (10 mL) was added t-BuN₃ (1.6 g, 21.9 mmol) and the mixture was stirred at 65° C. for 2 h under N₂. The mixture was cooled to room temperature, filtered and the filtrate was concentrated in vacuo to give the crude product which was purified by silica gel chromatography (eluent: DCM/MeOH=20/1) to give methyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (450 mg, 87.5%) as a yellow solid. ESI-MS [M+H]⁺: 472.2.

### Synthesis of tert-butyl ((6-cyclopropyl-8-(3-(2-hydroxy-2-methylpropyl)-2,4-dioxoimidazolidin-1-yl)imidazo[1,2-a]pyridin-2-yl)methyl)(methyl)carbamate

To a solution of methyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (300 mg, 0.63 mmol) in THF (10 mL) was added CH₃MgBr (2.1 mL, 3 M in THF, 6.3 mmol) at 0° C. and then mixture was stirred at room temperature for 40 min. The mixture was quenched with sat·NH₄Cl solution (50 mL) and extracted with EtOAc (3×30 mL). The combined organic layers were washed with brine (30 mL), dried over Na₂SO₄ and concentrated in vacuo. The residue was purified by preparative TLC (eluent: DCM/MeOH=16/1) to give tert-butyl ((6-cyclopropyl-8-(3-(2-hydroxy-2-methylpropyl)-2,4-dioxoimidazolidin-1-yl)imidazo[1,2-a]pyridin-2-yl)methyl)(methyl)carbamate (30 mg, 10%) as a yellow solid. ESI-MS [M+H]⁺: 472.2.

### Synthesis of 1-(6-cyclopropyl-2-((methylamino)methyl)imidazo[1,2-a]pyridin-8-yl)-3-(2-hydroxy-2-methylpropyl)imidazolidine-2,4-dione

To a solution of tert-butyl ((6-cyclopropyl-8-(3-(2-hydroxy-2-methylpropyl)-2,4-dioxoimidazolidin-1-yl)imidazo[1,2-a]pyridin-2-yl)methyl)(methyl)carbamate (30 mg, 0.063 mmol) in dioxane (2 mL) was added HCl/dioxane (2 mL) at room temperature and the mixture was stirred for 2 h. The mixture was concentrated in vacuo to give 1-(6-cyclopropyl-2-((methylamino)methyl)imidazo[1,2-a]pyridin-8-yl)-3-(2-hydroxy-2-methylpropyl)imidazolidine-2,4-dione (30 mg, crude) as a yellow solid which was used in next step without purification. ESI-MS [M+H]⁺: 372.2.

### Synthesis of 1-(2-(1-aminoethyl)-7-cyclopropylimidazo[1,2-a]pyridin-5-yl)-3-methylimidazolidine-2,4-dione

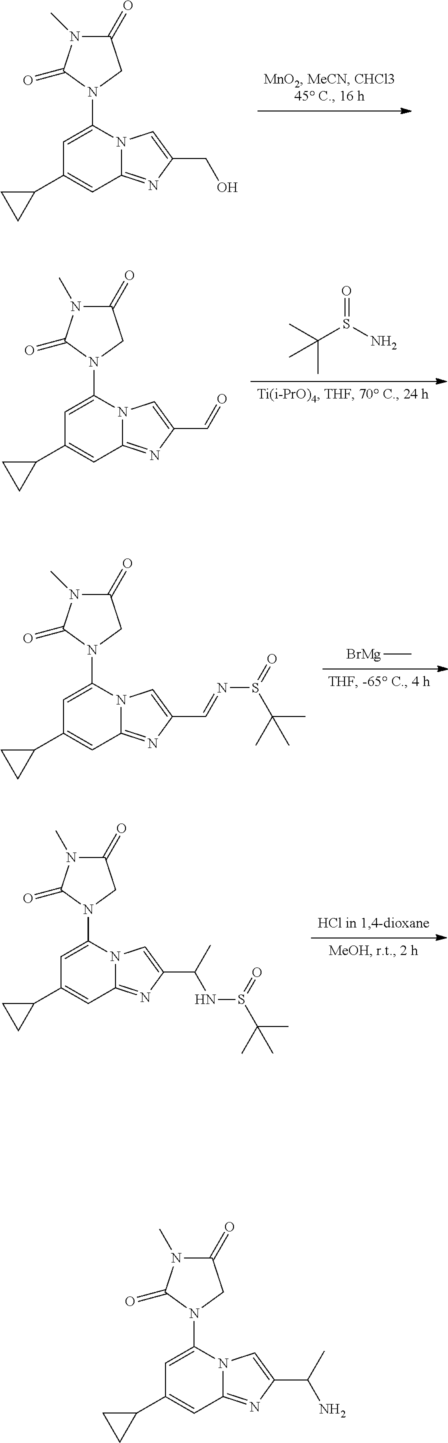

### Synthesis of 7-cyclopropyl-5-(3-methyl-2,4-dioxoimidazolidin-1-yl)imidazo[1,2-a]pyridine-2-carbaldehyde

A mixture of 1-(7-cyclopropyl-2-(hydroxymethyl)imidazo[1,2-a]pyridin-5-yl)-3-methylimidazolidine-2,4-dione (300 mg, 1.0 mmol) and MnO₂ (1.3 g, 15 mmol) in CHCl₃ (3.0 ml) and MeCN (3.0 mL) was stirred at 45° C. for 16 h. The reaction mixture was filtered through celite and the filter cake was washed with DCM/MeOH (10/1, 50 mL). The filtrate was concentrated in vacuo to give the crude, which was purified by silica gel chromatography (eluent: PE/EA=1/2) to give 7-cyclopropyl-5-(3-methyl-2,4-dioxoimidazolidin-1-yl)imidazo[1,2-a]pyridine-2-carbaldehyde (200 mg, 67%) as a yellow solid. ESI-MS: [M+H]⁺, 299.1

### Synthesis of (E)-N-((7-cyclopropyl-5-(3-methyl-2,4-dioxoimidazolidin-1-yl)imidazo[1,2-a]pyridin-2-yl)methylene)-2-methylpropane-2-sulfinamide

To a solution of 7-cyclopropyl-5-(3-methyl-2,4-dioxoimidazolidin-1-yl)imidazo[1,2-a]pyridine-2-carbaldehyde (180 mg, 0.6 mmol) and 2-methylpropane-2-sulfinamide (218 mg, 1.8 mmol) in THF (6 mL) was added Ti(i-PrO)₄ (1.02 g, 3.6 mmol) dropwise. The resulting mixture was stirred at 70° C. for another 24 h. The reaction was quenched with water (50 mL) then extracted with EtOAc (3×30 mL). The combined organic layers were washed with brine (20 mL), dried over anhydrous Na₂SO₄, and concentrated in vacuo to give the crude, which was purified by silica gel chromatography (eluent, DCM/MeOH=30/1) to give (E)-N-((7-cyclopropyl-5-(3-methyl-2,4-dioxoimidazolidin-1-yl)imidazo[1,2-a]pyridin-2-yl)methylene)-2-methylpropane-2-sulfinamide (100 mg, 41%) as a yellow solid. ESI-MS: [M+H]⁺, 402.1

### Synthesis of N-(1-(7-cyclopropyl-5-(3-methyl-2,4-dioxoimidazolidin-1-yl)imidazo[1,2-a]pyridin-2-yl)ethyl)-2-methylpropane-2-sulfinamide

A solution of (E)-N-((7-cyclopropyl-5-(3-methyl-2,4-dioxoimidazolidin-1-yl)imidazo[1,2-a]pyridin-2-yl)methylene)-2-methylpropane-2-sulfinamide (100 mg, 0.25 mmol) in THF (4 mL) was cooled to −65° C. Methylmagnesium bromide (3M solution in THF, 0.292 ml, 0.875 mmol) was added dropwise. The resulting mixture was stirred at −65° C. for 4 h under N₂. The reaction was quenched with saturated aqueous NH₄Cl (20 mL) then extracted with DCM (3×20 ml), The combined organic layers were washed with brine (20 mL), dried over anhydrous Na₂SO₄, and concentrated in vacuo to give the crude product, which was purified by silica gel chromatography (eluent: DCM/MeOH=25/1) to give N-(1-(7-cyclopropyl-5-(3-methyl-2,4-dioxoimidazolidin-1-yl)imidazo[1,2-a]pyridin-2-yl)ethyl)-2-methylpropane-2-sulfinamide (18 mg, 17%) as a yellow solid. ESI-MS: [M+H]⁺, 418.1

### Synthesis of 1-(2-(1-aminoethyl)-7-cyclopropylimidazo[1,2-a]pyridin-5-yl)-3-methylimidazolidine-2,4-dione

To a mixture of N-(1-(7-cyclopropyl-5-(3-methyl-2,4-dioxoimidazolidin-1-yl)imidazo[1,2-a]pyridin-2-yl)ethyl)-2-methylpropane-2-sulfinamide (50 mg, 0.12 mmol) in MeOH (1 mL) was added HCl (4M solution in dioxane, 0.5 mL). The resulting mixture was stirred at room temperature for 2 h. The reaction mixture was diluted with DCM (20 ml) and concentrated in vacuo to give the crude, which was purified by preparative TLC (eluent, DCM/MeOH=8/1) to give 1-(2-(1-aminoethyl)-7-cyclopropylimidazo[1,2-a]pyridin-5-yl)-3-methylimidazolidine-2,4-dione (20 mg, 53%) as a yellow solid. ESI-MS: [M+H]⁺, 314.1

### Synthesis of 1-(2-(1-aminoethyl)-6-cyclopropylimidazo[1,2-a]pyridin-8-yl)-3-methylimidazolidin-2-one

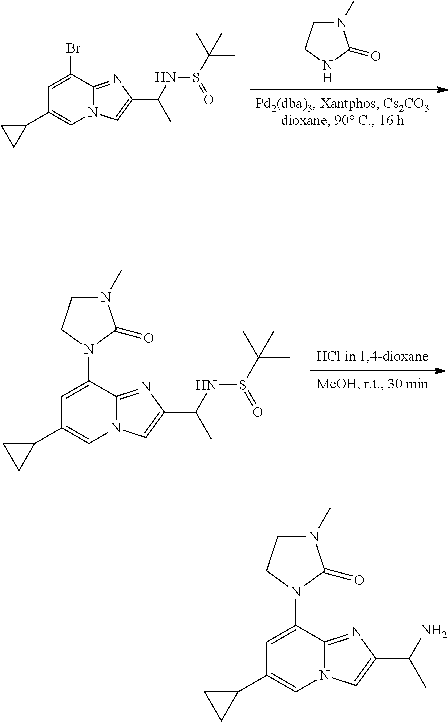

### Synthesis of N-(1-(6-cyclopropyl-8-(3-methyl-2-oxoimidazolidin-1-yl)imidazo[1,2-a]pyridin-2-yl)ethyl)-2-methylpropane-2-sulfinamide

To a mixture of N-(1-(8-bromo-6-cyclopropylimidazo[1,2-a]pyridin-2-yl)ethyl)-2-methylpropane-2-sulfinamide (200 mg, 0.52 mmol), 1-methylimidazolidin-2-one (156 mg, 1.56 mmol) and Cs₂CO₃ (509 mg, 1.56 mmol) in 1,4-dioxane (10 mL) were added Pd₂(dba)₃ (48 mg, 0.052 mmol) and Xantphos (60 mg, 0.104 mmol). The mixture was stirred at 90° C. for 16 h under N₂. The reaction mixture was cooled to room temperature, filtered through celite and the filter cake was washed with DCM/MeOH (10/1, 30 mL). The filtrate was concentrated in vacuo to give the crude product, which was purified by column chromatography (eluent: EtOAc/PE=1/2) to give N-(1-(6-cyclopropyl-8-(3-methyl-2-oxoimidazolidin-1-yl)imidazo[1,2-a]pyridin-2-yl)ethyl)-2-methylpropane-2-sulfinamide (180 mg, 86%) as a yellow solid. ESI-MS [M+H]⁺: 404.2.

### Synthesis of 1-(2-(1-aminoethyl)-6-cyclopropylimidazo[1,2-a]pyridin-8-yl)-3-methylimidazolidin-2-one

To a stirred solution of N-(1-(6-cyclopropyl-8-(3-methyl-2-oxoimidazolidin-1-yl)imidazo[1,2-a]pyridin-2-yl)ethyl)-2-methylpropane-2-sulfinamide (215 mg, 0.53 mmol) in MeOH (5 mL) was added HCl (4 M solution in 1,4-dioxane, 5 mL). The mixture was stirred at room temperature for 30 min. The reaction mixture was concentrated in vacuo and a solution of NH₃ (7 M solution in MeOH, 5 mL) was added. The resulting mixture was stirred for 10 min and concentrated in vacuo to give the crude, which was purified by preparative TLC (eluent: DCM/MeOH=10/1) to give 1-(2-(1-aminoethyl)-6-cyclopropylimidazo[1,2-a]pyridin-8-yl)-3-methylimidazolidin-2-one (110 mg, 69%) as a white solid. ESI-MS [M+H]⁺: 300.2.

### Synthesis of 1-(2-(1-aminoethyl)-6-cyclopropylimidazo[1,2-a]pyridin-8-yl)-3-(2-(benzyloxy)ethyl)imidazolidine-2,4-dione

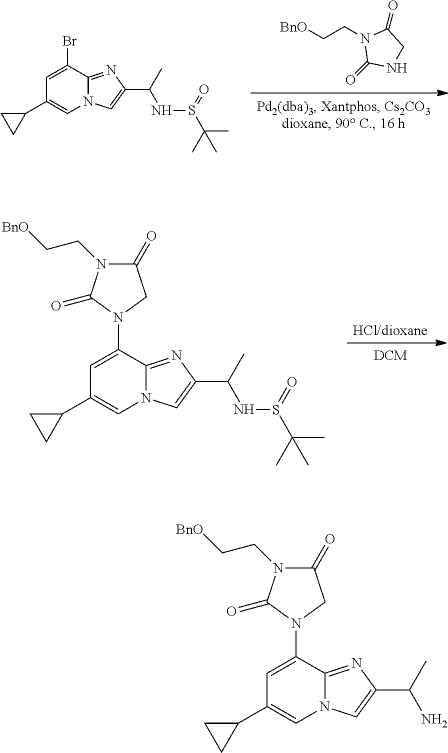

### Synthesis of N-(1-(8-(3-(2-(benzyloxy)ethyl)-2,4-dioxoimidazolidin-1-yl)-6-cyclopropylimidazo[1,2-a]pyridin-2-yl)ethyl)-2-methylpropane-2-sulfinamide

To a solution of N-(1-(8-bromo-6-cyclopropylimidazo[1,2-a]pyridin-2-yl)ethyl)-2-methylpropane-2-sulfinamide (300 mg, 0.78 mmol) in dioxane (10 mL) were added 3-(2-(benzyloxy)ethyl)imidazolidine-2,4-dione (548 mg, 2.34 mmol), Pd₂(dba)₃ (143 mg, 0.16 mmol), Xantphos (181 mg, 0.31 mmol) and Cs₂CO₃ (1019 mg, 3.13 mmol). After stirring the mixture at 95° C. for 16 h under N₂, it was cooled to room temperature, diluted with water (50 mL) and extracted with EtOAc (3×50 mL). The combined organic layers were washed with brine (50 mL), dried over Na₂SO₄ then concentrated to give the crude, which was purified by silica gel (eluant: DCM:MeOH=50:1) to give N-(1-(8-(3-(2-(benzyloxy)ethyl)-2,4-dioxoimidazolidin-1-yl)-6-cyclopropylimidazo[1,2-a]pyridin-2-yl)ethyl)-2-methylpropane-2-sulfinamide as yellow solid. (300 mg, 72% yield). ESI-MS [M+H]⁺: 538.2

### Synthesis of 1-(2-(1-aminoethyl)-6-cyclopropylimidazo[1,2-a]pyridin-8-yl)-3-(2-(benzyloxy)ethyl)imidazolidine-2,4-dione

To a solution of N-(1-(8-(3-(2-(benzyloxy)ethyl)-2,4-dioxoimidazolidin-1-yl)-6-cyclopropylimidazo[1,2-a]pyridin-2-yl)ethyl)-2-methylpropane-2-sulfinamide (300 mg, 0.55 mmol) in DCM (10 mL) was added HCl (4M solution in dioxane, 1 mL) and then the mixture was stirred at 0° C. for 1 h. The resulting solution was concentrated and basified by NH₃/MeOH (30 mL) and concentrated to dryness. The residue was purified by preparative TLC (DCM:NH₃/MeOH=12:1) to give 1-(2-(1-aminoethyl)-6-cyclopropylimidazo[1,2-a]pyridin-8-yl)-3-(2-(benzyloxy)ethyl)imidazolidine-2,4-dione as yellow solid. (170 mg, 71% yield). ESI-MS [M+H]⁺: 434.2

### Synthesis of (R)-((2-(1-aminoethyl)-6-cyclopropylimidazo[1,2-a]pyridin-8-yl)imino)dimethyl-λ⁶-sulfanone

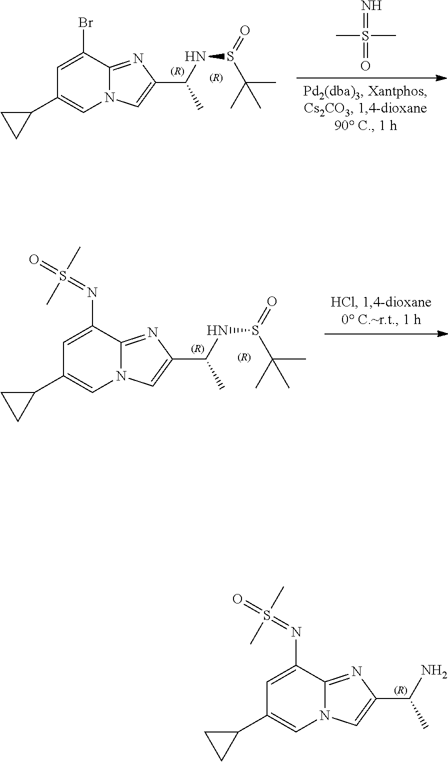

### Synthesis of (R)—N—((R)-1-(6-cyclopropyl-8-((dimethyl(oxo)-λ⁶-sulfaneylidene)amino)imidazo[1,2-a]pyridin-2-yl)ethyl)-2-methylpropane-2-sulfinamide

To a mixture of (R)—N—((R)-1-(8-bromo-6-cyclopropylimidazo[1,2-a]pyridin-2-yl)ethyl)-2-methylpropane-2-sulfinamide (350 mg, 0.91 mmol), iminodimethyl-λ⁶-sulfanone (213.9 mg, 2.3 mmol) and Cs₂CO₃ (743 mg, 2.28 mmol) in 1,4-dioxane (20 mL) was added Pd₂(dba)₃ (0.091 mmol) and Xantphos (104 mg, 0.18 mmol). The reaction mixture was stirred at 90° C. for 1 h and cooled to room temperature. The resulting solution was filtered through Celite® and the filter cake was washed with DCM/MeOH (10/1, 50 mL). The filtrate was concentrated in vacuo to give the crude, which was purified by preparative TLC (eluent: DCM/MeOH=20/1) to give (R)—N—((R)-1-(6-cyclopropyl-8-((dimethyl(oxo)-λ⁶-sulfaneylidene)amino)imidazo[1,2-a]pyridin-2-yl)ethyl)-2-methylpropane-2-sulfinamide (210 mg, 58%) as a white solid.

ESI-MS [M+H]+: 397.2

### Synthesis of (R)-((2-(1-aminoethyl)-6-cyclopropylimidazo[1,2-a]pyridin-8-yl)imino)dimethyl-λ⁶-sulfanone

To a solution of (R)—N—((R)-1-(6-cyclopropyl-8-((dimethyl(oxo)-λ⁶-sulfaneylidene)amino)imidazo[1,2-a]pyridin-2-yl)ethyl)-2-methylpropane-2-sulfinamide (210 mg, 0.53 mmol) in 1,4-dioxane (10 mL) was added HCl (4M solution in 1,4-dioxane, 1 mL) at 0° C. The reaction was stirred at room temperature for 1 h and concentrated in vacuo to give the crude, which was purified by preparative TLC (DCM/MeOH/NH₄OH=10/1/0.1) to give (R)-((2-(1-aminoethyl)-6-cyclopropylimidazo[1,2-a]pyridin-8-yl)imino)dimethyl-λ⁶-sulfanone (140 mg, 90%) as a yellow solid. ESI-MS [M+H]+: 293.1

### Synthesis of (R)-1-(2-(1-aminoethyl)-6-cyclopropylimidazo[1,2-a]pyridin-8-yl)-3,5,5-trimethylimidazolidine-2,4-dione

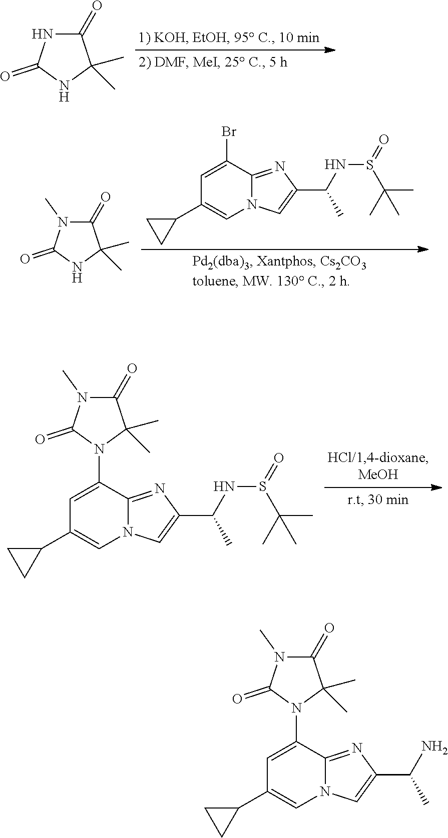

### Synthesis of 3,5,5-trimethylimidazolidine-2,4-dione

A mixture of 5,5-dimethylimidazolidine-2,4-dione (4.0 g, 0.031 mol) and KOH (1.9 g, 0.034 mol) were dissolved in EtOH (60 mL) by stirring at 95° C. for 10 min. After removing the solvent by evaporation, the resulting hydantoin potassium salt was dried under vacuum at 45° C. The residue was dissolved in DMF (60 mL) and then was added MeI (4.4 g, 0.031 mol). After stirring at 25° C. for 5 h, the reaction mixture was filtered, and the filtrate was concentrated in vacuo to give the crude product, which was purified by silica gel chromatography (eluent: PE/EtOAc=10/1) to give 3,5,5-trimethylimidazolidine-2,4-dione (0.50 g, 11%) as a yellow solid. ESI-MS [M+H]⁺: 143.0.

### Synthesis of N—((R)-1-(6-cyclopropyl-8-(3,5,5-trimethyl-2,4-dioxoimidazolidin-1-yl)imidazo[1,2-a]pyridin-2-yl)ethyl)-2-methylpropane-2-sulfinamide

A mixture of 3,5,5-trimethylimidazolidine-2,4-dione (0.44 g, 3.1 mmol), N—((R)-1-(8-bromo-6-cyclopropylimidazo[1,2-a]pyridin-2-yl)ethyl)-2-methylpropane-2-sulfinamide (0.30 g, 0.78 mmol), Pd₂(dba)₃ (0.15 g, 0.16 mmol), Xantphos (0.18 g, 0.31 mmol) and Cs₂CO₃ (0.75 g, 2.3 mmol) in toluene (10 mL) was stirred in a sealed tube. After degassing with N₂ for 1 min, the reaction was irradiated in microwave at 130° C. for 2 h. The reaction mixture was diluted in DCM/MeOH (50 mL, 10/1), filtered and the filtrate was concentrated in vacuo. The residue was purified by silica gel chromatography (eluent: PE/EtOAc=10/1) to give N—((R)-1-(6-cyclopropyl-8-(3,5,5-trimethyl-2,4-dioxoimidazolidin-1-yl)imidazo[1,2-a]pyridin-2-yl)ethyl)-2-methylpropane-2-sulfinamide (0.13 g, 37%) as a yellowy solid. ESI-MS [M+H]⁺: 446.2.

### Synthesis of (R)-1-(2-(1-aminoethyl)-6-cyclopropylimidazo[1,2-a]pyridin-8-yl)-3,5,5-trimethylimidazolidine-2,4-dione

To a solution of N—((R)-1-(6-cyclopropyl-8-(3,5,5-trimethyl-2,4-dioxoimidazolidin-1-yl)imidazo[1,2-a]pyridin-2-yl)ethyl)-2-methylpropane-2-sulfinamide (0.13 g, 0.29 mmol) in MeOH (2.0 mL) was added HCl (4.0 M solution in 1,4-dioxane, 3.0 mL). The resulting mixture was stirred at room temperature for 30 min and concentrated in vacuo. Then a solution of NH₃ (7 M in MeOH 5.0 mL) was added to the residue and stirred for 10 min. The mixture was concentrated in vacuo to give the crude, which was purified by preparative TLC (eluent: DCM/NH₃ (7 M in MeOH)=10/1) to give (R)-1-(2-(1-aminoethyl)-6-cyclopropylimidazo[1,2-a]pyridin-8-yl)-3,5,5-trimethylimidazolidine-2,4-dione (80 mg, 80%) as a white solid. ESI-MS [M+H]⁺: 342.2.

### Synthesis of (R)-1-(2-(1-aminoethyl)-6-cyclopropylimidazo[1,2-a]pyridin-8-yl)imidazolidine-2,4-dione

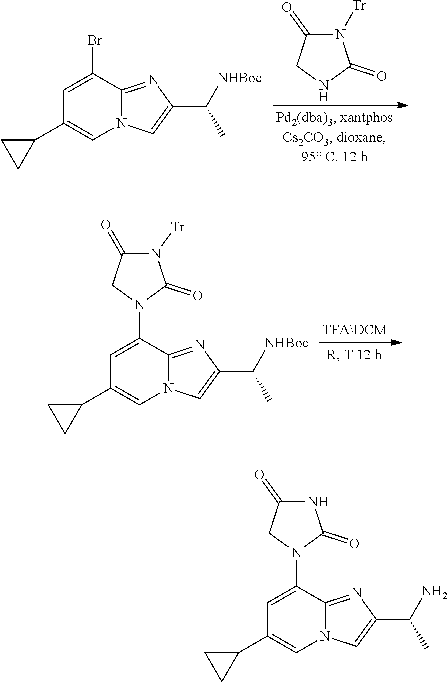

### Synthesis of tert-butyl (R)-(1-(6-cyclopropyl-8-(2,4-dioxo-3-tritylimidazolidin-1-yl)imidazo[1,2-a]pyridin-2-yl)ethyl)carbamate

To a mixture of tert-butyl (R)-(1-(8-bromo-6-cyclopropylimidazo[1,2-a]pyridin-2-yl)ethyl)carbamate (500 mg, 1.3 mmol), 3-tritylimidazolidine-2,4-dione (889 mg, 2.6 mmol) and Cs₂CO₃ (1.27 g, 3.9 mmol) in dioxane (10 mL) was added Pd₂(dba)₃ (174 mg, 0.19 mmol) and xantPhos (150 mg, 0.26 mmol). The reaction mixture was stirred at 95° C. for 12 h under N₂. Then the reaction mixture was filtered through Celite® and the filter cake was washed with DCM/MeOH (10/1, 100 mL). The filtrate was concentrated in vacuo to give the crude, which was purified by preparative TLC (eluent: DCM/MeOH=20/1) to give tert-butyl (R)-(1-(6-cyclopropyl-8-(2,4-dioxo-3-tritylimidazolidin-1-yl)imidazo[1,2-a]pyridin-2-yl)ethyl)carbamate (300 mg, 36%) as a yellow solid. ESI-MS [M+H]+: 642.2.

### Synthesis of (R)-1-(2-(1-aminoethyl)-6-cyclopropylimidazo[1,2-a]pyridin-8-yl)imidazolidine-2,4-dione

A solution of tert-butyl (R)-(1-(6-cyclopropyl-8-(2,4-dioxo-3-tritylimidazolidin-1-yl)imidazo[1,2-a]pyridin-2-yl)ethyl)carbamate (300 mg, 0.47 mmol) in TFA (2 mL) and DCM (2 mL) was stirred at room temperature for 12 h. The mixture was quenched with NaHCO₃ (sat·aq·, 50 mL) and extracted with DCM (50 mL×3). The combined organic layers were washed with brine (50 mL), dried over Na₂SO₄ and concentrated in vacuo. The residue was purified by column chromatography (eluent: DCM/MeOH=0-10%) to give (R)-1-(2-(1-aminoethyl)-6-cyclopropylimidazo[1,2-a]pyridin-8-yl)imidazolidine-2,4-dione (100 mg, 71%) as a white solid. ESI-MS [M+H]⁺: 300.2

### Synthesis of (R)-3-(2-(1-aminoethyl)-6-cyclopropylimidazo[1,2-a]pyridin-8-yl)oxazolidin-2-one

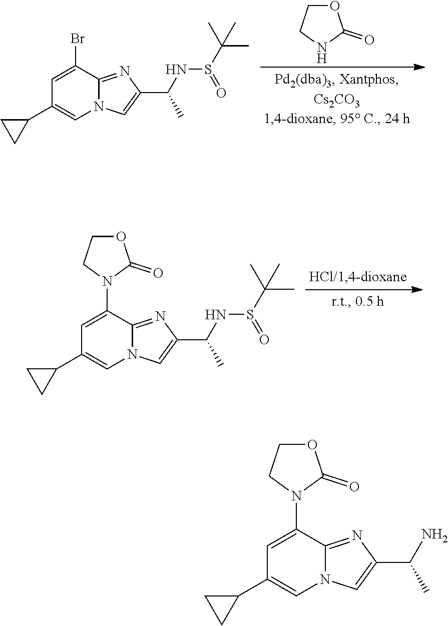 [SMILES: C[C@@H](NS(=O)C(C)(C)C)c1cn2cc(C3CC3)cc(Br)c2n1 ; O=C1NCCO1 ; C[C@@H](NS(=O)C(C)(C)C)c1cn2cc(C3CC3)cc(N3CCOC3=O)c2n1 ; C[C@@H](N)c1cn2cc(C3CC3)cc(N3CCOC3=O)c2n1]

### Synthesis of N—((R)-1-(6-cyclopropyl-8-(2-oxooxazolidin-3-yl)imidazo[1,2-a]pyridin-2-yl)ethyl)-2-methylpropane-2-sulfinamide

To a mixture of N—((R)-1-(8-bromo-6-cyclopropylimidazo[1,2-a]pyridin-2-yl)ethyl)-2-methylpropane-2-sulfinamide (295 mg, 0.78 mmol), oxazolidin-2-one (205 mg, 2.34 mmol) and Cs₂CO₃ (750 mg, 2.3 mmol) and Xantphos (93 mg, 0.16 mmol) in 1,4-dioxane (15 mL) was added Pd₂(dba)₃ (73 mg, 0.080 mmol) and the reaction mixture was stirred at 95° C. for 24 h under N₂. The reaction mixture was filtered through Celite® and the filter cake was washed with DCM/MeOH (10/1, 100 mL). The filtrate was concentrated in vacuo to give the crude, which was purified by column chromatography (eluent: EtOAc/PE=0-100%) to afford N—((R)-1-(6-cyclopropyl-8-(2-oxooxazolidin-3-yl)imidazo[1,2-a]pyridin-2-yl)ethyl)-2-methylpropane-2-sulfinamide (240 mg, 79%) as an off-yellow solid. ESI-MS [M+H]+: 391.2

### Synthesis of (R)-3-(2-(1-aminoethyl)-6-cyclopropylimidazo[1,2-a]pyridin-8-yl)oxazolidin-2-one

To a mixture of N—((R)-1-(6-cyclopropyl-8-(3-methyl-2-oxoimidazolidin-1-yl)imidazo[1,2-a]pyridin-2-yl)ethyl)-2-methylpropane-2-sulfinamide (240 mg, 0.62 mmol) in MeOH (3 mL) was added HCl/1,4-dioxane (5 mL, 4.0 M in 1,4-dioxane). The reaction was stirred at room temperature for 0.5 h. The mixture was quenched with NaHCO₃ (sat·aq·, 50 mL) and extracted with EtOAc (50 mL×3). The combined organic layers were washed with brine (50 mL), dried over Na₂SO₄ and concentrated in vacuo. The residue was purified by column chromatography (eluent: MeOH/DCM=0-10%) with to afford (R)-3-(2-(1-aminoethyl)-6-cyclopropylimidazo[1,2-a]pyridin-8-yl)oxazolidin-2-one (140 mg, 79%) as an yellow solid. ESI-MS [M+H]+: 287.1

### Synthesis of 1-(6-cyclopropyl-2-(1-(methylamino)ethyl)imidazo[1,2-a]pyridin-8-yl)-3-methylimidazolidine-2,4-dione

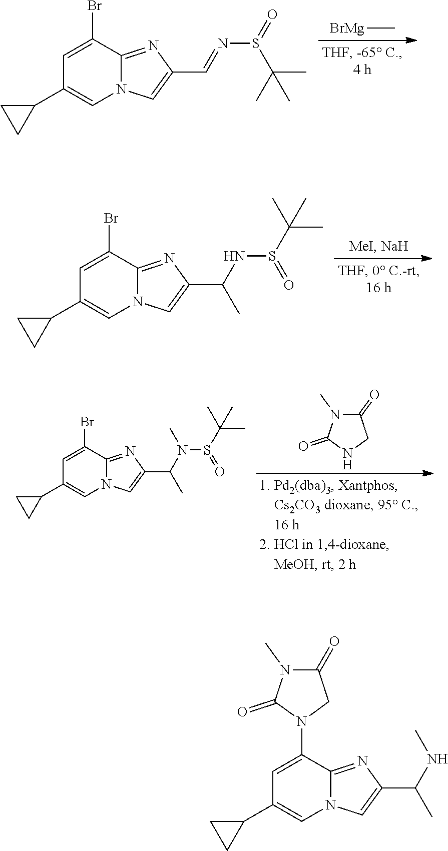

### Synthesis of N-(1-(8-bromo-6-cyclopropylimidazo[1,2-a]pyridin-2-yl)ethyl)-2-methylpropane-2-sulfinamide

To a mixture of (E)-N-((8-bromo-6-cyclopropylimidazo[1,2-a]pyridin-2-yl)methylene)-2-methylpropane-2-sulfinamide (1 g, 2.7 mmol) in THF (25 mL) was added CH₃MgBr (2.7 mL, 3 M in THF, 8.1 mmol) at −65° C. and then stirred for 4 h under N₂. Then the mixture was quenched with sat·NH₄Cl solution (20 mL) at −65° C., warmed to room temperature and extracted with DCM (3×30 mL). The combined organic layers were washed with brine (20 mL), dried over anhydrous Na₂SO₄, and concentrated in vacuo to give N-(1-(8-bromo-6-cyclopropylimidazo[1,2-a]pyridin-2-yl)ethyl)-2-methylpropane-2-sulfinamide (860 mg, crude) as a yellow solid. ESI-MS [M+H]⁺: 384.2.

### Synthesis of N-(1-(8-bromo-6-cyclopropylimidazo[1,2-a]pyridin-2-yl)ethyl)-N,2-dimethylpropane-2-sulfinamide

To a solution of N-(1-(8-bromo-6-cyclopropylimidazo[1,2-a]pyridin-2-yl)ethyl)-2-methylpropane-2-sulfinamide (500 mg, crude) in THF (20 mL) was added NaH (76 mg, 1.9 mmol) at 0° C. The mixture was stirred for 0.5 h. Then a solution of MeI (269.8 mg, 1.9 mmol) in THF (2 mL) was added to the reaction mixture and it was stirred at room temperature for 16 h. The reaction was quenched with water (10 mL) and extracted with EtOAc (3×20 mL). The combined organic layers were washed with brine (20 mL), dried over anhydrous Na₂SO₄ and concentrated to give the crude product, which was purified by preparative TLC (eluent: DCM/MeOH=10/1) to give methyl 2-(3-(6-cyclopropyl-2-(hydroxymethyl)imidazo[1,2-a]pyridin-8-yl)-2,5-dioxoimidazolidin-1-yl)acetate (370 mg, 58% in 2 steps) as a yellow solid. ESI-MS [M+H]⁺: 400.1.

### Synthesis of N-(1-(6-cyclopropyl-8-(3-methyl-2,4-dioxoimidazolidin-1-yl)imidazo[1,2-a]pyridin-2-yl)ethyl)-N,2-dimethylpropane-2-sulfinamide

To a solution of methyl 2-(3-(6-cyclopropyl-2-(hydroxymethyl)imidazo[1,2-a]pyridin-8-yl)-2,5-dioxoimidazolidin-1-yl)acetate (370 mg, 0.93 mmol) in dioxane (10 mL) was added 3-methylimidazolidine-2,4-dione (424 mg, 3.72 mmol), Pd₂(dba)₃ (164.9 mg, 0.18 mmol), Xantphos (208.1 mg, 0.36 mmol) and Cs₂CO₃ (909.5 mg, 2.79 mmol). After stirring the mixture at 95° C. for 16 h under N₂, the mixture was filtered through celite and the filter cake was washed with DCM/MeOH (10/1, 50 mL). The filtrate was concentrated in vacuo to give the crude product, which was purified by silica gel chromatography (eluent: DCM/MeOH=50/1˜20/1) to give N-(1-(6-cyclopropyl-8-(3-methyl-2,4-dioxoimidazolidin-1-yl)imidazo[1,2-a]pyridin-2-yl)ethyl)-N,2-dimethylpropane-2-sulfinamide (300 mg, 75%) as a yellow solid. ESI-MS [M+H]⁺: 432.2.

### Synthesis of 1-(6-cyclopropyl-2-(1-(methylamino)ethyl)imidazo[1,2-a]pyridin-8-yl)-3-methylimidazolidine-2,4-dione

To a solution of N-(1-(6-cyclopropyl-8-(3-methyl-2,4-dioxoimidazolidin-1-yl)imidazo[1,2-a]pyridin-2-yl)ethyl)-N,2-dimethylpropane-2-sulfinamide (300 mg, 0.70 mmol) in MeOH (5 mL) was added HCl (4M solution in dioxane, 5 mL). After stirring for 2 h, the reaction mixture was concentrated in vacuo to give 1-(6-cyclopropyl-2-(1-(methylamino)ethyl)imidazo[1,2-a]pyridin-8-yl)-3-methylimidazolidine-2,4-dione as the hydrochloric acid salt (200 mg, 79%) as a yellow solid which was used in next step without purification. ESI-MS [M+H]+: 328.1.

### Synthesis of 1-(2-(aminomethyl-d2)-6-cyclopropylimidazo[1,2-a]pyridin-8-yl)-3-methylimidazolidine-2,4-dione

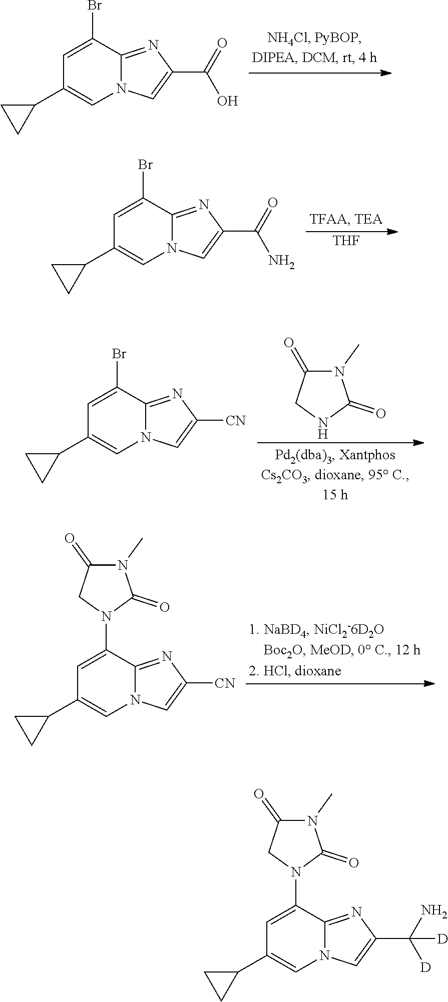

### Synthesis of 8-bromo-6-cyclopropylimidazo[1,2-a]pyridine-2-carboxamide

A solution of 8-bromo-6-cyclopropylimidazo[1,2-a]pyridine-2-carboxylic acid (1.12 g, crude), NH₄Cl (424 mg, 8 mmol), PyBOP (2796 mg, 6 mmol) and DIPEA (2064 mg, 16 mmol) in DCM (30 mL) was stirred at room temperature for 3 h. The reaction mixture was diluted with DCM (50 mL), washed with sat·NaHCO₃ solution (50 mL) and water (50 mL). The organic layer was dried over anhydrous Na₂SO₄ and concentrated in vacuo to get the crude, which was purified by silica gel chromatography (eluent: DCM/MeOH=10/1) to afford 8-bromo-6-cyclopropylimidazo[1,2-a]pyridine-2-carboxamide (840 mg, 75%) as yellow solid. ESI-MS [M+H]⁺: 280.0.

### Synthesis of 8-bromo-6-cyclopropylimidazo[1,2-a]pyridine-2-carbonitrile

To a solution of 8-bromo-6-cyclopropylimidazo[1,2-a]pyridine-2-carboxamide (560 mg, 2 mmol) in TEA (2 ml) and THF (10 mL) was added TFAA (1 mL) at 0° C. After stirring for 2 h, the reaction was quenched with water (30 mL) and extracted with EtOAc (3×30 mL). The combined organic layers were washed with brine (20 mL), dried over anhydrous Na₂SO₄, and concentrated to give the crude product, which was purified by column chromatography (eluent: DCM/MeOH=30/1) to give the 8-bromo-6-cyclopropylimidazo[1,2-a]pyridine-2-carbonitrile (290 mg, 56%) as yellow solid. ESI-MS [M+H]+: 262.0.

### Synthesis of 6-cyclopropyl-8-(3-methyl-2,4-dioxoimidazolidin-1-yl)imidazo[1,2-a]pyridine-2-carbonitrile

To a mixture of 8-bromo-6-cyclopropylimidazo[1,2-a]pyridine-2-carbonitrile (290 mg, 1.1 mmol) and 3-methylimidazolidine-2,4-dione (376 mg, 3.3 mmol) and Cs₂CO₃ (1076 mg, 3.3 mmol) in dioxane (20 mL) was added Pd₂(dba)₃ (100 mg, 0.11 mmol) and Xantphos (127 mg, 0.22 mmol). The reaction was stirred at 95° C. under N₂ for 15 h, cooled to room temperature, filtered through celite and the filter cake was washed with DCM/MeOH (10/1, 50 mL). The filtrate was concentrated in vacuo to give the crude, which was purified by preparative TLC (eluent: DCM/MeOH=20/1) to give the 6-cyclopropyl-8-(3-methyl-2,4-dioxoimidazolidin-1-yl)imidazo[1,2-a]pyridine-2-carbonitrile (180 mg, 56%) as yellow solid. ESI-MS [M+H]+: 296.1.

### Synthesis of tert-butyl((6-cyclopropyl-8-(3-methyl-2,4-dioxoimidazolidin-1-yl)imidazo[1,2-a]pyridin-2-yl)methyl-d2)carbamate

To a mixture of 6-cyclopropyl-8-(3-methyl-2,4-dioxoimidazolidin-1-yl)imidazo[1,2-a]pyridine-2-carbonitrile (180 mg, 0.61 mmol) and NiCl₂ 6D₂O (7 mg, 0.03 mmol) in MeOD (10 mL) was added Boc₂O (266 mg, 1.22 mmol). After stirring at room temperature under N₂ for 30 min, the reaction mixture was treated with NaBD₄ (154 mg, 3.66 mmol) slowly in small portions. The mixture was stirred at 0° C. under N₂ for 1 h and then at room temperature for 12 h. The reaction was quenched with sat·aqueous NH₄Cl solution (30 mL) and extracted with ethyl acetate (3×30 mL). The combined organic layers were dried over anhydrous Na₂SO₄ and concentrated in vacuo to give the crude, which was purified by preparative TLC (eluent: DCM/MeOH=15/1) to give the tert-butyl((6-cyclopropyl-8-(3-methyl-2,4-dioxoimidazolidin-1-yl)imidazo[1,2-a]pyridin-2-yl)methyl-d2)carbamate (100 mg, 41%) as yellow solid. ESI-MS [M+H]+: 402.2.

### Synthesis of 1-(2-(aminomethyl-d2)-6-cyclopropylimidazo[1,2-a]pyridin-8-yl)-3-methylimidazolidine-2,4-dione

To a solution of tert-butyl ((6-cyclopropyl-8-(3-methyl-2,4-dioxoimidazolidin-1-yl)imidazo[1,2-a]pyridin-2-yl)methyl-d2)carbamate (100 mg, 0.25 mol) in dioxane (2 mL). was added HCl (4M solution in dioxane, 2 mL). After stirring at room temperature for 1 h., the mixture was basified (pH 8) with sat·aqueous NaHCO₃ solution and the mixture was extracted with DCM (3×20 mL). The combined organic layers were washed with brine (30 mL), dried over anhydrous Na₂SO₄ and concentrated in vacuo to give the crude product, which was purified by preparative TLC (eluent: DCM/MeOH=10/1) to afford 1-(2-(aminomethyl-d2)-6-cyclopropylimidazo[1,2-a]pyridin-8-yl)-3-methylimidazolidine-2,4-dione (55 mg, 73%) as a white solid. ESI-MS [M+H]+: 302.1.

### Synthesis of (R)-1-(2-(1-aminoethyl)-6-cyclopropylimidazo[1,2-a]pyridin-8-yl)-3-methylimidazolidine-2,4-dione

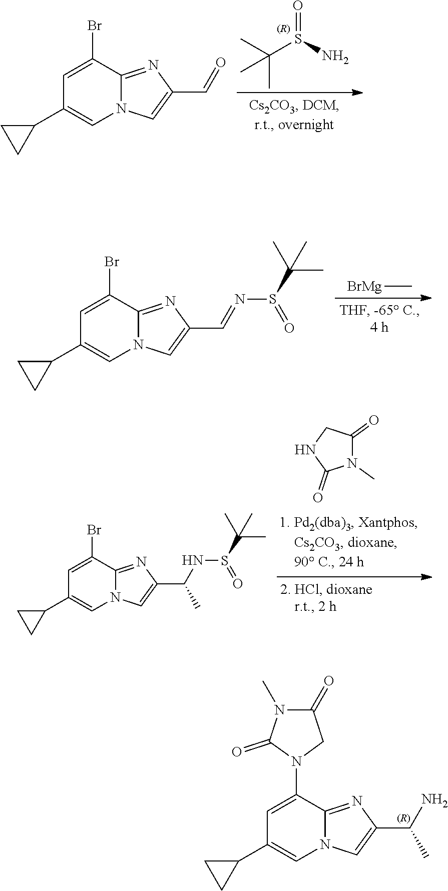

### Synthesis of (R)—N-((6-cyclopropylimidazo[1,2-a]pyridin-2-yl)methylene)-2-methylpropane-2-sulfinamide

To a solution of 8-bromo-6-cyclopropylimidazo[1,2-a]pyridine-2-carbaldehyde (4.2 g, 15.8 mmol) in DCM (80 mL) was added (R)-2-methylpropane-2-sulfinamide (2.5 g, 0.21 mmol) and Cs₂CO₃ (10.33 g, 0.21 mmol). The reaction mixture was stirred at room temperature overnight, then poured into water (100 mL) and extracted with EtOAc (100 mL×3). The combined organics were washed with brine (100 mL), dried over Na₂SO₄. The reaction mixture was concentrated in vacuo and purified by silica gel chromatography (eluent: PE:EtOAc=5:1) to give (R)—N-((8-bromo-6-cyclopropylimidazo[1,2-a]pyridin-2-yl)methylene)-2-methylpropane-2-sulfinamide (4.2 g, yield 72%). ESI-MS [M+H]+: 368.0

### Synthesis of (R)—N—((R)-1-(8-bromo-6-cyclopropylimidazo[1,2-a]pyridin-2-yl)ethyl)-2-methylpropane-2-sulfinamide

To a solution of (R,E)-N-((6-cyclopropylimidazo[1,2-a]pyridin-2-yl)methylene)-2-methylpropane-2-sulfinamide (200 mg, 0.54 mmol) in THF (5 mL) was added methylmagnesium bromide (0.72 mL, 2.16 mmol) at −65° C. and the mixture was stirred for 4 h at the same temperature. The reaction mixture was quenched with NH₄Cl solution (sat·aq·, 50 mL) at −65° C., stirred for 10 min then extracted with EtOAc (50 mL×3). The combined organics were washed with brine (50 mL), dried over Na₂SO₄ and concentrated in vacuo. The residue was purified by silica gel chromatography (eluent: PE:EtOAc=5:1) to give (R)—N—((R)-1-(8-bromo-6-cyclopropylimidazo[1,2-a]pyridin-2-yl)ethyl)-2-methylpropane-2-sulfinamide (160 mg, yield 77%). ESI-MS [M+H]+: 384.1

### Synthesis of (R)—N—((R)-1-(6-cyclopropyl-8-(3-methyl-2,4-dioxoimidazolidin-1-yl)imidazo[1,2-a]pyridin-2-yl)ethyl)-2-methylpropane-2-sulfinamide

To a solution of (R)—N—((R)-1-(8-bromo-6-cyclopropylimidazo[1,2-a]pyridin-2-yl)ethyl)-2-methylpropane-2-sulfinamide (400 mg, 1.05 mmol) in 1,4-dioxane (40 mL) were added 3-methylimidazolidine-2,4-dione (190 mg, 1.56 mmol), Pd₂(dba)₃ (100 mg, 0.105 mmol), XantPhos (120 mg, 0.21 mmol), and Cs₂CO₃ (1.15 g, 3.09 mmol) at 90° C. for 16 h under N₂. The reaction mixture was filtered and the filter cake was washed with DCM/MeOH (10/1, 50 mL). The filtrate was concentrated in vacuo to give the crude residue, which was purified by silica gel chromatography (eluent: DCM/MeOH=50/1˜20/1) to give (R)—N—((R)-1-(6-cyclopropyl-8-(3-methyl-2,4-dioxoimidazolidin-1-yl)imidazo[1,2-a]pyridin-2-yl)ethyl)-2-methylpropane-2-sulfinamide (400 mg, 91%). ESI-MS [M+H]+: 418.2

### Synthesis of (R)-1-(2-(1-aminoethyl)-6-cyclopropylimidazo[1,2-a]pyridin-8-yl)-3-methylimidazolidine-2,4-dione

A mixture of (R)—N—((R)-1-(6-cyclopropyl-8-(3-methyl-2,4-dioxoimidazolidin-1-yl)imidazo[1,2-a]pyridin-2-yl)ethyl)-2-methylpropane-2-sulfinamide (70 mg, 0.17 mmol) in HCl/dioxane (4M, 2 mL) was stirred at room temperature for 1 h. The reaction mixture was poured into ammonia in methanol (7M, 2 mL) and concentrated in vacuo to give the crude residue, which was purified by silica gel chromatography (eluent: DCM:MeOH=10:1) to give (R)-1-(2-(1-aminoethyl)-6-cyclopropylimidazo[1,2-a]pyridin-8-yl)-3-methylimidazolidine-2,4-dione a white solid (50 mg, yield 95%). ESI-MS [M+H]+: 314.

### Synthesis of (R)-1-(2-(1-aminopropyl)-6-cyclopropylimidazo[1,2-a]pyridin-8-yl)-3-methyl imidazolidine-2,4-dione

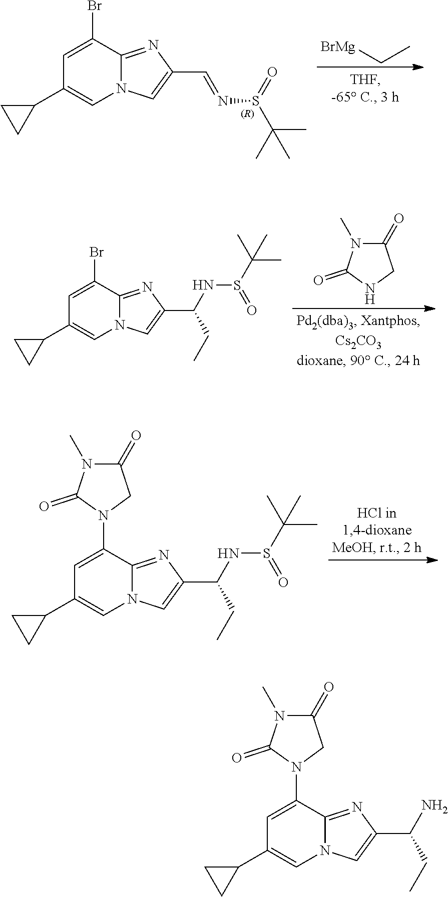

Using a similar procedure to that for ((R)-1-(2-(1-aminoethyl)-6-cyclopropylimidazo[1,2-a]pyridin-8-yl)-3-methylimidazolidine-2,4-dione, (R)-1-(2-(1-aminopropyl)-6-cyclopropylimidazo[1,2-a]pyridin-8-yl)-3-methylimidazolidine-2,4-dione (243 mg) was synthesised from (R,E)-N-((8-bromo-6-cyclopropylimidazo[1,2-a]pyridin-2-yl)methylene)-2-methylpropane-2-sulfinamide and ethylmagnesium bromide. ESI-MS [M+H]+: 328.1

### Synthesis of 3-(2-((R)-1-aminoethyl)-6-cyclopropylimidazo[1,2-a]pyridin-8-yl)-3-azabicyclo[3.1.0]hexan-2-one

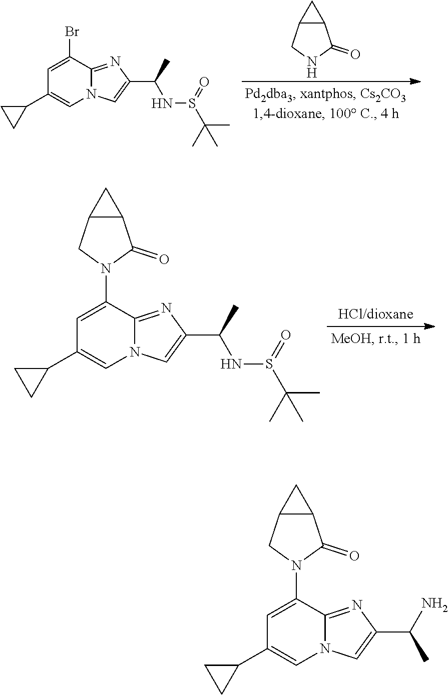 [SMILES: C[C@@H](NS(=O)C(C)(C)C)c1cn2cc(C3CC3)cc(Br)c2n1 ; O=C1NCC2CC12 ; C[C@@H](NS(=O)C(C)(C)C)c1cn2cc(C3CC3)cc(N3CC4CC4C3=O)c2n1 ; C[C@H](N)c1cn2cc(C3CC3)cc(N3CC4CC4C3=O)c2n1]

### Synthesis of N-((1R)-1-(6-cyclopropyl-8-(2-oxo-3-azabicyclo[3.1.0]hexan-3-yl)imidazo[1,2-a]pyridin-2-yl)ethyl)-2-methylpropane-2-sulfinamide

To a mixture of N—((R)-1-(8-bromo-6-cyclopropylimidazo[1,2-a]pyridin-2-yl)ethyl)-2-methylpropane-2-sulfinamide (0.75 g, 1.9 mmol), 3-azabicyclo[3.1.0]hexan-2-one (0.19 g, 1.9 mmol) and Cs₂CO₃ (1.9 g, 5.7 mmol) in 1,4-dioxane (30 mL) was added Pd₂(dba)₃ (0.36 g, 0.38 mmol) and Xantphos (0.45 g, 0.76 mmol). The resulting mixture was stirred at 100° C. for 4 h under N₂ and cooled to room temperature. The reaction mixture was filtered through Celite® and the filter cake was washed with DCM/MeOH (10/1, 50 mL). The filtrate was concentrated in vacuo to give the crude, which was purified by silica gel column chromatography (eluent: DCM/MeOH=0˜5%) to afford N-((1R)-1-(6-cyclopropyl-8-(2-oxo-3-azabicyclo[3.1.0]hexan-3-yl)imidazo[1,2-a]pyridin-2-yl)ethyl)-2-methylpropane-2-sulfinamide (0.76 g, 97%) as a yellow solid. ESI-MS: [M+H]⁺, 401.2

### Synthesis of 3-(2-((R)-1-aminoethyl)-6-cyclopropylimidazo[1,2-a]pyridin-8-yl)-3-azabicyclo[3.1.0]hexan-2-one

To a mixture of N-((1R)-1-(6-cyclopropyl-8-(2-oxo-3-azabicyclo[3.1.0]hexan-3-yl)imidazo[1,2-a]pyridin-2-yl)ethyl)-2-methylpropane-2-sulfinamide (0.76 g, 1.9 mmol) in MeOH (10 mL) was added HCl (5 mL, 20 mmol, 4M in 1,4-dioxane,) at 0° C. The reaction mixture was stirred at room temperature for 1 h and concentrated in vacuo. The resulting residue was basified (pH=8) with NH₃/MeOH (5 mL, 35 mmol, 7M in MeOH) at 0° C. The mixture was diluted with DCM (90 mL), filtered and the filter cake was washed by DCM (3×40 ml). The filtrate was concentrated in vacuo to give the crude, which was purified by column chromatography (eluent: MeOH/DCM=1/8) to afford 3-(2-((R)-1-aminoethyl)-6-cyclopropylimidazo[1,2-a]pyridin-8-yl)-3-azabicyclo[3.1.0]hexan-2-one (0.54 g, 96%) as a yellow solid. ESI-MS: [M+H]⁺, 297.1

### Synthesis of (R)-1-(2-(1-aminoethyl)-6-cyclopropylimidazo[1,2-a]pyridin-8-yl)pyrrolidin-2-one

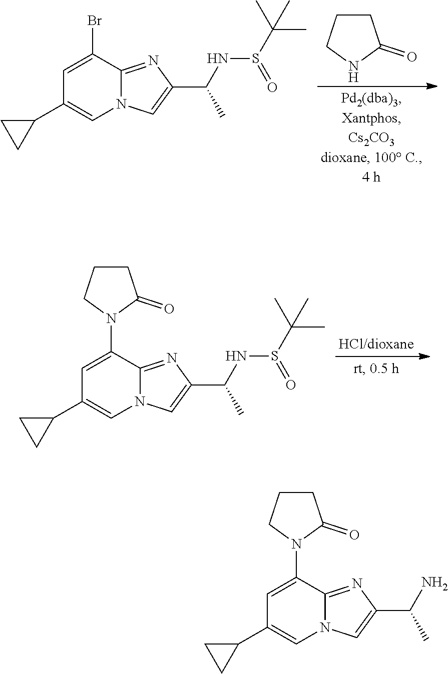

### Synthesis of N—((R)-1-(6-cyclopropyl-8-(2-oxopyrrolidin-1-yl)imidazo[1,2-a]pyridin-2-yl)ethyl)-2-methylpropane-2-sulfinamide

A mixture of N—((R)-1-(8-bromo-6-cyclopropylimidazo[1,2-a]pyridin-2-yl)ethyl)-2-methylpropane-2-sulfinamide (300 mg, 0.78 mmol), pyrrolidin-2-one (266 mg, 3.12 mmol), Pd₂(dba)₃ (143 mg, 0.156 mmol), XantPhos (180 mg, 0.312 mmol) and Cs₂CO₃ (763 mg, 2.34 mmol) in dioxane (15 mL) was stirred at 100° C. for 4 h under N₂. The reaction mixture was filtered through Celite® and the filter cake was washed with DCM/MeOH (10/1, 50 mL). The filtrate was concentrated in vacuo to give the crude, which was purified by column chromatography (eluent: DCM/MeOH=0˜10/1) to give N—((R)-1-(6-cyclopropyl-8-(2-oxopyrrolidin-1-yl)imidazo[1,2-a]pyridin-2-yl)ethyl)-2-methylpropane-2-sulfinamide (250 mg, 83%) as a yellow oil. ESI-MS [M+H]+: 389.2

### Synthesis of (R)-1-(2-(1-aminoethyl)-6-cyclopropylimidazo[1,2-a]pyridin-8-yl)pyrrolidin-2-one

To a mixture of N—((R)-1-(6-cyclopropyl-8-(2-oxopyrrolidin-1-yl)imidazo[1,2-a]pyridin-2-yl)ethyl)-2-methylpropane-2-sulfinamide (250 mg, 0.64 mmol) in MeOH (3 mL) was added HCl (5 mL, 4M solution in dioxane). The resulting mixture was stirred at room temperature for 0.5 h. The reaction mixture was concentrated in vacuo to give (R)-1-(2-(1-aminoethyl)-6-cyclopropylimidazo[1,2-a]pyridin-8-yl)pyrrolidin-2-one as the hydrochloric acid salt (250 mg, quant) as a yellow solid. ESI-MS [M+H]+: 285.1.

### Synthesis of 1-(2-(1-amino-2,2,2-trifluoroethyl)-6-cyclopropylimidazo[1,2-a]pyridin-8-yl)-3-methylimidazolidine-2,4-dione

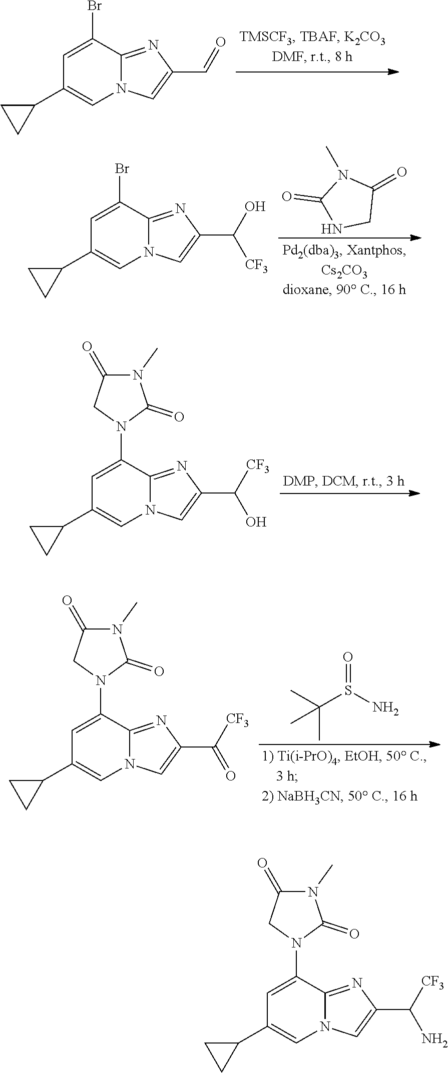

### Synthesis of 1-(8-bromo-6-cyclopropylimidazo[1,2-a]pyridin-2-yl)-2,2,2-trifluoroethan-1-ol

To a solution of 8-bromo-6-cyclopropylimidazo[1,2-a]pyridine-2-carbaldehyde (1.5 g, 5.68 mmol) in DMF (30 ml) were added K₂CO₃ (78 mg, 0.568 mmol) and TMSCF₃ (1.2 g, 8.52 mmol). The resulting reaction was stirred at room temperature for 4 h, then TBAF (8.52 mL, 8.52 mmol) was added dropwise. After stirring for another 4 h, the mixture was quenched with water (200 mL) and extracted with DCM (3×100 mL). The combined organic layers were washed with brine (200 mL), dried over anhydrous Na₂SO₄, and concentrated in vacuo to give the crude, which was purified by silica gel chromatography (eluent: PE/EtOAc=1/1) to give 1-(8-bromo-6-cyclopropylimidazo[1,2-a]pyridin-2-yl)-2,2,2-trifluoroethan-1-ol (1.2 g, 63%) as a yellow solid. ESI-MS: [M+H]⁺, 335.2.

### Synthesis of 1-(6-cyclopropyl-2-(2,2,2-trifluoro-1-hydroxyethyl)imidazo[1,2-a]pyridin-8-yl)-3-methylimidazolidine-2,4-dione

To a solution of 1-(8-bromo-6-cyclopropylimidazo[1,2-a]pyridin-2-yl)-2,2,2-trifluoroethan-1-ol (1.0 g, 2.99 mmol), 3-methylimidazolidine-2,4-dione (1.0 g, 8.97 mmol) and Cs₂CO₃ (2.4 g, 7.48 mmol) in 1,4-dioxane (30 mL) was added Pd₂(dba)₃ (274 mg, 0.299 mmol) and Xantphos (346 mg, 0.598 mmol). After stirring at 90° C. for 16 h under N₂, the reaction mixture was cooled to room temperature, filtered through celite and the filter cake was washed with DCM/MeOH (10/1, 100 mL). The filtrate was concentrated in vacuo to give the crude, which was purified by silica gel chromatography (eluent: DCM/MeOH=20/1) to give 1-(6-cyclopropyl-2-(2,2,2-trifluoro-1-hydroxyethyl)imidazo[1,2-a]pyridin-8-yl)-3-methylimidazolidine-2,4-dione (800 mg, 73%) as a yellow solid. ESI-MS: [M+H]⁺, 369.1.

### Synthesis of 1-(6-cyclopropyl-2-(2,2,2-trifluoroacetyl)imidazo[1,2-a]pyridin-8-yl)-3-methyl imidazolidine-2,4-dione

To a solution of 1-(6-cyclopropyl-2-(2,2,2-trifluoro-1-hydroxyethyl)imidazo[1,2-a]pyridin-8-yl)-3-methylimidazolidine-2,4-dione (800 mg, 2.17 mmol) in DCM (20 mL) was added DMP (2.76 g, 6.51 mmol). The resulting mixture was stirred at room temperature for 3 h and then quenched with saturated aqueous Na₂S₂O₃ solution (30 mL) and extracted with DCM (3×50 mL). The combined organic layers were washed with brine (80 mL), dried over anhydrous Na₂SO₄, and concentrated in vacuo to give the crude, which was purified by silica gel chromatography (eluent: DCM/MeOH=25/1) to give 1-(6-cyclopropyl-2-(2,2,2-trifluoroacetyl)imidazo[1,2-a]pyridin-8-yl)-3-methylimidazolidine-2,4-dione (550 mg, 69%) as a yellow solid. ESI-MS: [M+H]⁺, 367.2.

### Synthesis of 1-(2-(1-amino-2,2,2-trifluoroethyl)-6-cyclopropylimidazo[1,2-a]pyridin-8-yl)-3-methylimidazolidine-2,4-dione

To a solution of 1-(6-cyclopropyl-2-(2,2,2-trifluoroacetyl)imidazo[1,2-a]pyridin-8-yl)-3-methylimidazolidine-2,4-dione (350 mg, 0.96 mmol) and 2-methylpropane-2-sulfinamide (1.74 g, 14.40 mmol) in EtOH (20 mL) was added Ti(i-PrO)₄ (2.73 g, 9.6 mmol) dropwise. The resulting reaction was stirred at 50° C. for 3 h, and then NaBH₃CN (363 mg, 5.76 mmol) was added. The resulting mixture was stirred at 50° C. for another 16 h. The reaction was quenched with water (30 mL) then extracted with DCM (3×30 mL). The combined organic layers were washed with brine (50 mL), dried over anhydrous Na₂SO₄, and concentrated in vacuo to give the crude, which was purified by silica gel chromatography (eluent: DCM/MeOH=10/1) to give 1-(2-(1-amino-2,2,2-trifluoroethyl)-6-cyclopropylimidazo[1,2-a]pyridin-8-yl)-3-methylimidazolidine-2,4-dione (150 mg, 42%) as a white solid. ESI-MS: [M+H]⁺, 368.1.

### Synthesis of 1-(2-(1-amino-2-methoxyethyl)-6-cyclopropylimidazo[1,2-a]pyridin-8-yl)-3-methylimidazolidine-2,4-dione

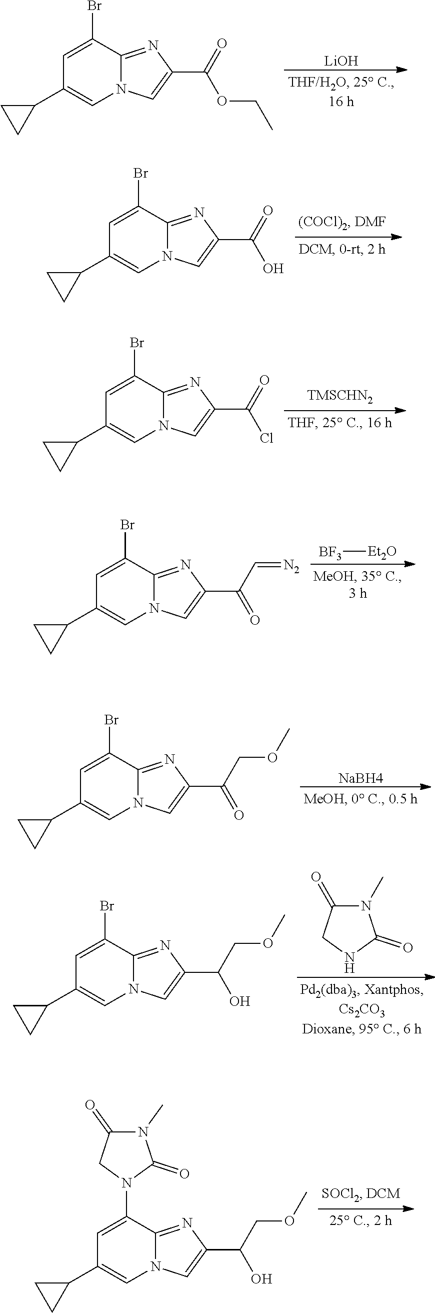

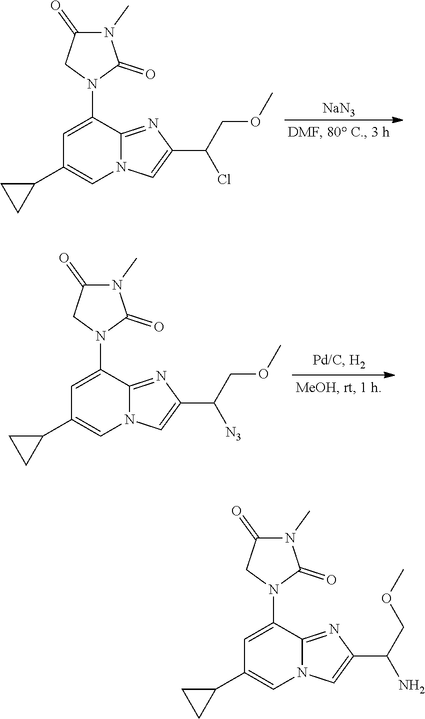 [SMILES: COCC(Cl)c1cn2cc(C3CC3)cc(N3CC(=O)N(C)C3=O)c2n1 ; COCC(N=[N+]=[N-])c1cn2cc(C3CC3)cc(N3CC(=O)N(C)C3=O)c2n1 ; COCC(N)c1cn2cc(C3CC3)cc(N3CC(=O)N(C)C3=O)c2n1 ; "NaN3"]

### Synthesis of 8-bromo-6-cyclopropylimidazo[1,2-a]pyridine-2-carboxylic acid

To a solution of ethyl 8-bromo-6-cyclopropylimidazo[1,2-a]pyridine-2-carboxylate (3.25 g, 10.51 mmol) in THF (20 mL), water (20 mL), and EtOH (4 mL) was added LiOH·H₂O (882 mg, 21.02 mmol). After stirring at 25° C. for 16 h, the resulting mixture was concentrated to give the crude, which was diluted with water (10 mL) and then acidified to pH 5 by adding HCl (1M aq·). The aqueous layer was extracted with EtOAc (3×30 mL), washed with brine (30 mL), dried over Na₂SO₄ and concentrated in vacuo to give 8-bromo-6-cyclopropylimidazo[1,2-a]pyridine-2-carboxylic acid (2 g, 68%) as a white solid. ESI-MS [M+H]⁺: 281.0.

### Synthesis of 8-bromo-6-cyclopropylimidazo[1,2-a]pyridine-2-carbonyl chloride

To a solution of 8-bromo-6-cyclopropylimidazo[1,2-a]pyridine-2-carboxylic acid (2 g, 7.11 mmol) in DCM (50 mL) was added oxalyl chloride (1.8 g, 14.18 mmol) and catalytic amount of DMF at 0° C. After stirring at 25° C. for 2 h, the resulting mixture was concentrated to give 8-bromo-6-cyclopropylimidazo[1,2-a]pyridine-2-carbonyl chloride (2 g). This crude product was used for next step without further purification.

### Synthesis of 1-(8-bromo-6-cyclopropylimidazo[1,2-a]pyridin-2-yl)-2-diazoethan-1-one

To a solution of 8-bromo-6-cyclopropylimidazo[1,2-a]pyridine-2-carbonyl chloride (2 g, crude) in THF (15 mL) was added TMSCHN₂ (10 mL, 2 M solution in hexane) at 0° C. After stirring at 25° C. for 16 h, the resulting mixture was concentrated to give 1-(8-bromo-6-cyclopropylimidazo[1,2-a]pyridin-2-yl)-2-diazoethan-1-one (2 g) as a colorless oil. This crude product was used to next step without further purification. ESI-MS [M+H]+: 305.1.

### Synthesis of 1-(8-bromo-6-cyclopropylimidazo[1,2-a]pyridin-2-yl)-2-methoxyethan-1-one

To a solution of 1-(8-bromo-6-cyclopropylimidazo[1,2-a]pyridin-2-yl)-2-diazoethan-1-one (2 g, crude) in MeOH was added boron trifluoride etherate (0.6 mL, 48% BF₃) at 0° C. After stirring at 35° C. for 3 h, the resulting mixture was concentrated in vacuo to give the crude, which was purified by silica gel chromatography (eluent: EtOAc/PE=0˜100/1) to give 1-(8-bromo-6-cyclopropylimidazo[1,2-a]pyridin-2-yl)-2-methoxyethan-1-one (900 mg, 41%) as a yellow solid. ESI-MS [M+H]+: 309.0.

### Synthesis of 1-(8-bromo-6-cyclopropylimidazo[1,2-a]pyridin-2-yl)-2-methoxyethan-1-ol

To a solution of 1-(8-bromo-6-cyclopropylimidazo[1,2-a]pyridin-2-yl)-2-methoxyethan-1-one (450 mg, 1.46 mmol) in MeOH (110 mL) was added NaBH₄ (110 mg, 2.91 mmol) at 0° C. After stirring at 25° C. for 1 h, the resulting mixture was quenched with saturated aqueous NH₄Cl (20 mL) and extracted with EtOAc (3×20 mL). The combined organic layers were washed with brine (20 mL), dried over Na₂SO₄, and concentrated in vacuo to give the crude product, which was purified by silica gel chromatography (eluent: EtOAc/PE=0˜100/1) to give 1-(8-bromo-6-cyclopropylimidazo[1,2-a]pyridin-2-yl)-2-methoxyethan-1-ol (200 mg, 44%) as a colorless oil. ESI-MS [M+H]+: 311.0.

### Synthesis of 1-(6-cyclopropyl-2-(1-hydroxy-2-methoxyethyl)imidazo[1,2-a]pyridin-8-yl)-3-methylimidazolidine-2,4-dione

To a mixture of 1-(8-bromo-6-cyclopropylimidazo[1,2-a]pyridin-2-yl)-2-methoxyethan-1-ol (150 mg, 0.48 mmol), 3-methylimidazolidine-2,4-dione (82 mg, 0.72 mmol), and Cs₂CO₃ (470 mg, 1.44 mmol) in dioxane (5 mL) were added Pd₂(dba)₃ (82 mg, 0.090 mmol) and Xantphos (110 mg, 0.190 mmol). The reaction mixture was stirred at 95° C. for 16 h under N₂ and, after cooling to room temperature, the mixture was filtered through celite and the filter cake was washed with EtOAc (20 mL). The filtrate was concentrated to give the crude product, which was purified by column chromatography (eluent: EtOAc/PE=0˜100/1) to give 1-(6-cyclopropyl-2-(1-hydroxy-2-methoxyethyl)imidazo[1,2-a]pyridin-8-yl)-3-methylimidazolidine-2,4-dione (120 mg, 73% yield) as a yellow solid, ESI-MS [M+H]+: 345.2.

### Synthesis of 1-(2-(1-chloro-2-methoxyethyl)-6-cyclopropylimidazo[1,2-a]pyridin-8-yl)-3-methylimidazolidine-2,4-dione

To a solution of 1-(6-cyclopropyl-2-(1-hydroxy-2-methoxyethyl)imidazo[1,2-a]pyridin-8-yl)-3-methylimidazolidine-2,4-dione (100 mg, 0.29 mmol) in DCM (5 mL) was added SOCl₂ (1.0 mL) at 0° C. After stirring at 25° C. for 2 h, the resulting mixture was concentrated to give the 1-(2-(1-chloro-2-methoxyethyl)-6-cyclopropylimidazo[1,2-a]pyridin-8-yl)-3-methylimidazolidine-2,4-dione (100 mg). The crude colorless oil was used for next step directly without further purification.

### Synthesis of 1-(2-(1-azido-2-methoxyethyl)-6-cyclopropylimidazo[1,2-a]pyridin-8-yl)-3-methylimidazolidine-2,4-dione

To a solution of 1-(2-(1-chloro-2-methoxyethyl)-6-cyclopropylimidazo[1,2-a]pyridin-8-yl)-3-methylimidazolidine-2,4-dione (100 mg, crude) in DMF (5 mL) was added NaN₃ (63 mg, 0.99 mmol) at room temperature and the reaction mixture was stirred at 80° C. for 3 h. The reaction was cooled to room temperature, quenched with water (30 mL) and extracted with EtOAc (3×30 mL). The combined organic layers were concentrated in vacuo to give 1-(2-(1-azido-2-methoxyethyl)-6-cyclopropylimidazo[1,2-a]pyridin-8-yl)-3-methylimidazolidine-2,4-dione (100 mg, crude) as a yellow oil. ESI-MS [M+H]⁺: 370.1.

### Synthesis of 1-(2-(1-amino-2-methoxyethyl)-6-cyclopropylimidazo[1,2-a]pyridin-8-yl)-3-methylimidazolidine-2,4-dione

To a solution of 1-(2-(1-azido-2-methoxyethyl)-6-cyclopropylimidazo[1,2-a]pyridin-8-yl)-3-methylimidazolidine-2,4-dione (100 mg, crude) in MeOH (5 mL) was added Pd/C (10 mg) at room temperature and the reaction mixture was stirred for 1 h. The mixture was then filtered and the filtrate was concentrated in vacuo to give 1-(2-(1-amino-2-methoxyethyl)-6-cyclopropylimidazo[1,2-a]pyridin-8-yl)-3-methylimidazolidine-2,4-dione (60 mg, crude) as a yellow oil which was used in next step without purification. ESI-MS [M+H]⁺: 344.1.

### Synthesis of 1-(2-(1-amino-2-hydroxyethyl)-6-cyclopropylimidazo[1,2-a]pyridin-8-yl)-3-methylimidazolidine-2,4-dione

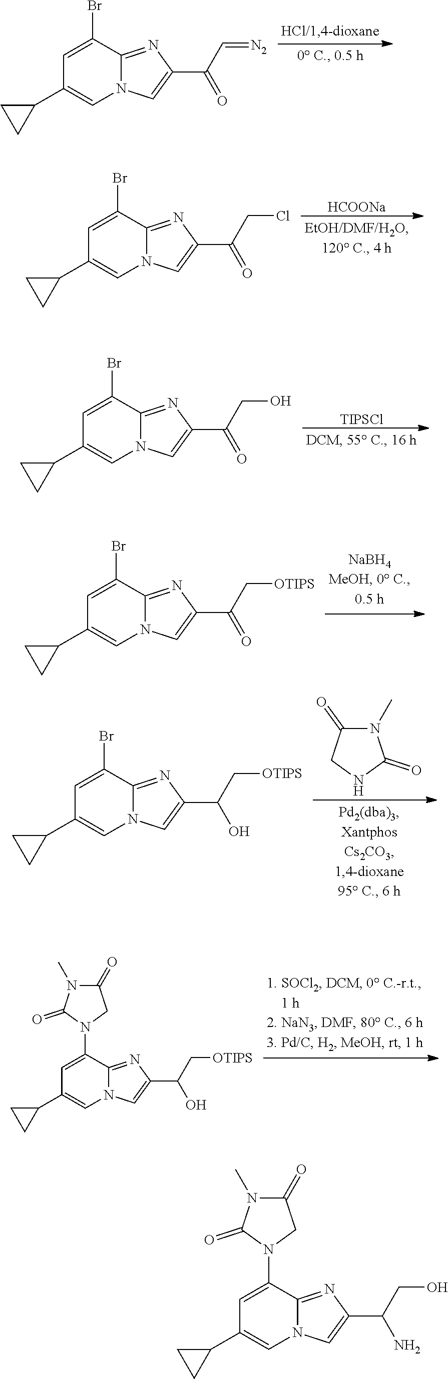

### Synthesis of 1-(8-bromo-6-cyclopropylimidazo[1,2-a]pyridin-2-yl)-2-chloroethan-1-one

To a mixture of 1-(8-bromo-6-cyclopropylimidazo[1,2-a]pyridin-2-yl)-2-diazoethan-1-one (2.1 g, 7.1 mmol) in THF (50 mL) was added HCl (4M solution in dioxane, 5 mL) at 0° C. and stirred for 0.5 h. The reaction mixture was concentrated in vacuo to give the crude, which was purified by column chromatography (eluent: PE/EtOAc=2/1) to give 1-(8-bromo-6-cyclopropylimidazo[1,2-a]pyridin-2-yl)-2-chloroethan-1-one (1.0 g, 45%) as a yellow solid. ESI-MS [M+H]+: 313.0.

### Synthesis of 1-(8-bromo-6-cyclopropylimidazo[1,2-a]pyridin-2-yl)-2-hydroxyethan-1-one

A mixture of 1-(8-bromo-6-cyclopropylimidazo[1,2-a]pyridin-2-yl)-2-chloroethan-1-one (1.1 g, 3.5 mmol), HCOONa (0.72 g, 11 mmol) in a solution of EtOH (28 mL), DMF (10 mL) and water (8 mL) was stirred at 120° C. for 4h. After cooling to 25° C., water (40 mL) was added, the reaction mixture was extracted by DCM (3×40 mL). The organic layers were concentrated in vacuo to give the crude, which was purified by silica gel chromatography (eluent: PE/EtOAc=2/1) to give 1-(8-bromo-6-cyclopropylimidazo[1,2-a]pyridin-2-yl)-2-hydroxyethan-1-one (0.54 g, 52%) as a pale solid. ESI-MS [M+H]+: 295.1.

### Synthesis of 1-(8-bromo-6-cyclopropylimidazo[1,2-a]pyridin-2-yl)-2-((triisopropylsilyl)oxy)ethan-1-one

To a mixture of 1-(8-bromo-6-cyclopropylimidazo[1,2-a]pyridin-2-yl)-2-hydroxyethan-1-one (0.54 g, 1.8 mmol) and imidazole (0.87 g, 13 mmol) in DCM (30 mL) was added TIPSCl (1.8 g, 9.2 mmol). The reaction was stirred at 55° C. for 16 h under N₂. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give the crude, which was purified by silica gel chromatography (eluent: PE/EtOAc=2/1) to give 1-(8-bromo-6-cyclopropylimidazo[1,2-a]pyridin-2-yl)-2-((triisopropylsilyl)oxy)ethan-1-one (0.65 g, 80%) as a pale solid. ESI-MS [M+H]+: 451.1.

### Synthesis of 1-(2-(1-amino-2-hydroxyethyl)-6-cyclopropylimidazo[1,2-a]pyridin-8-yl)-3-methylimidazolidine-2,4-dione

Using a similar procedure to that for 1-(2-(1-amino-2-methoxyethyl)-6-cyclopropylimidazo[1,2-a]pyridin-8-yl)-3-methylimidazolidine-2,4-dione, 1-(2-(1-amino-2-hydroxyethyl)-6-cyclopropylimidazo[1,2-a]pyridin-8-yl)-3-methylimidazolidine-2,4-dione (170 mg, crude) was synthesised from 1-(8-bromo-6-cyclopropylimidazo[1,2-a]pyridin-2-yl)-2-((triisopropylsilyl)oxy)ethan-1-one. ESI-MS [M+H]+: 330.1

### Synthesis of 1-(2-(chloromethyl)-6-cyclopropylimidazo[1,2-a]pyridin-8-yl)-3-methylimidazolidine-2,4-dione

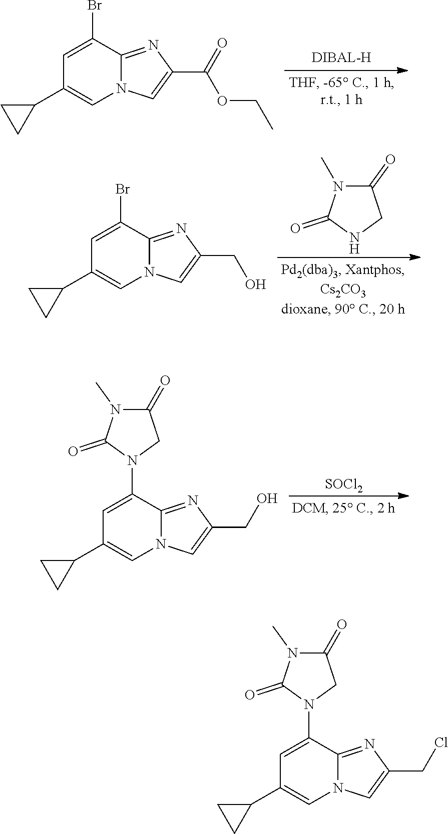

### Synthesis ethyl 8-bromo-6-cyclopropylimidazo[1,2-a]pyridine-2-carboxylate

A mixture of 3-bromo-5-cyclopropylpyridin-2-amine (4.9 g, 23 mmol), ethyl 3-bromo-2-oxopropanoate (5.4 g, 28 mmol) in DME (100 mL) was stirred at 90° C. for 24 h under N₂. After cooling to room temperature, the reaction was quenched with saturated aq·Na₂CO₃ (20 mL) and extracted with EtOAc (3×200 mL). The combined organic layers were washed with saturated brine solution (20 mL), dried over anhydrous Na₂SO₄, and concentrated in vacuo to give the crude, which was purified by column chromatography on silica gel (eluent: PE/EtOAc=1/1) to give ethyl 8-bromo-6-cyclopropylimidazo[1,2-a]pyridine-2-carboxylate (4.0 g, 56%) as a yellow solid. ESI-MS [M+H]⁺: 309.1

### Synthesis of (8-bromo-6-cyclopropylimidazo[1,2-a]pyridin-2-yl)methanol

A mixture of ethyl 8-bromo-6-cyclopropylimidazo[1,2-a]pyridine-2-carboxylate (4.0 g, 13 mmol) in THF (60 mL) was added DIBAL-H (1M in hexane, 40 mL, 40 mmol) dropwise. The resulting mixture was stirred at −65° C. for 1 h and then warmed to 25° C. and stirred for another 1 h under N₂. The reaction was quenched with NaOH (aqueous 10%, 30 mL) and extracted with EtOAc (4×50 mL). The combined organic layers were washed with saturated brine solution (30 mL), dried over anhydrous Na₂SO₄, and concentrated in vacuo to give the crude, which was purified by column chromatography on silica gel (eluent: PE/EtOAc=3/1) to give (8-bromo-6-cyclopropylimidazo[1,2-a]pyridin-2-yl)methanol (2.0 g, 58%) as a yellow solid. ESI-MS [M+H]+: 267.1

### Synthesis of 1-(6-cyclopropyl-2-(hydroxymethyl)imidazo[1,2-a]pyridin-8-yl)-3-methylimidazolidine-2,4-dione

To a mixture of (8-bromo-6-cyclopropylimidazo[1,2-a]pyridin-2-yl)methanol (0.86 g, 3.2 mmol), 3-methylimidazolidine-2,4-dione (0.74 g, 6.5 mmol) and Cs₂CO₃ (2.6 g, 8.0 mmol) in dioxane (12 mL) were added Pd₂(dba)₃ (0.30 g, 0.33 mmol) and Xantphos (0.37 g, 0.64 mmol). The reaction mixture was stirred at 90° C. for 20 h under N₂. After cooling to room temperature, the reaction mixture was filtered through Celite® and the filter cake was washed with DCM/MeOH (10/1, 100 mL). The filtrate was concentrated in vacuo to give the crude, which was purified by column chromatography on silica gel (eluent: MeOH/DCM=1/20) to give 1-(6-cyclopropyl-2-(hydroxymethyl)imidazo[1,2-a]pyridin-8-yl)-3-methylimidazolidine-2,4-dione (0.44 g, 46%) as a yellow solid. ESI-MS [M+H]+: 300.1.

### Synthesis of 1-(2-(chloromethyl)-6-cyclopropylimidazo[1,2-a]pyridin-8-yl)-3-methylimidazolidine-2,4-dione

To a solution of 1-(6-cyclopropyl-2-(hydroxymethyl)imidazo[1,2-a]pyridin-8-yl)-3-methylimidazolidine-2,4-dione (0.40 g, 1.3 mmol) in DCM (10 mL) was added SOCl₂ (3.0 mL). The resulting mixture was stirred at 25° C. for 2 h under N₂. The reaction mixture was concentrated in vacuo to give 1-(2-(chloromethyl)-6-cyclopropylimidazo[1,2-a]pyridin-8-yl)-3-methylimidazolidine-2,4-dione (0.42 g, quant) as a yellow solid which was used to the next step without further purification. ESI-MS [M+H]+: 319.1.

### Synthesis of 6-cyclopropyl-8-(3-methyl-2,4-dioxoimidazolidin-1-yl)imidazo[1,2-a]pyridine-2-carbaldehyde

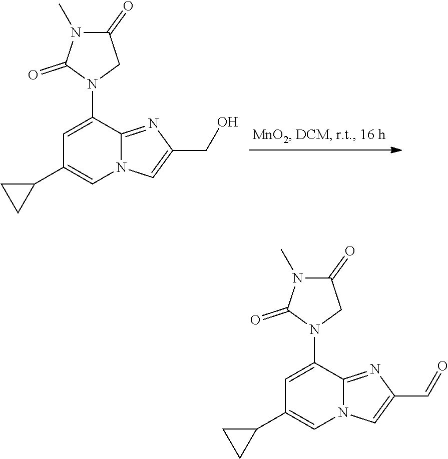 [SMILES: CN1C(=O)CN(c2cc(C3CC3)cn3cc(CO)nc23)C1=O ; CN1C(=O)CN(c2cc(C3CC3)cn3cc(C=O)nc23)C1=O]

A mixture of 1-(6-cyclopropyl-2-(hydroxymethyl)imidazo[1,2-a]pyridin-8-yl)-3-methylimidazolidine-2,4-dione (0.30 g, 1.0 mmol) and MnO₂ (0.87 g, 10 mmol) in DCM (10.0 mL) was stirred at room temperature for 16 h. The reaction mixture was filtered through Celite® and the filter cake was washed with DCM/MeOH (50/1, 50 mL). The filtrate was concentrated in vacuo to give the crude, which was purified by column chromatography on silica gel (eluent: DCM/MeOH=50/1˜20/1) to give 6-cyclopropyl-8-(3-methyl-2,4-dioxoimidazolidin-1-yl)imidazo-[1,2-a]pyridine-2-carbaldehyde (0.22 g, 74%) as a yellow solid. ESI-MS [M+H]⁺: 299.2.

### Synthesis of 1-(6-cyclopropyl-2-(1-hydroxyethyl)imidazo[1,2-a]pyridin-8-yl)-3-methylimidazolidine-2,4-dione

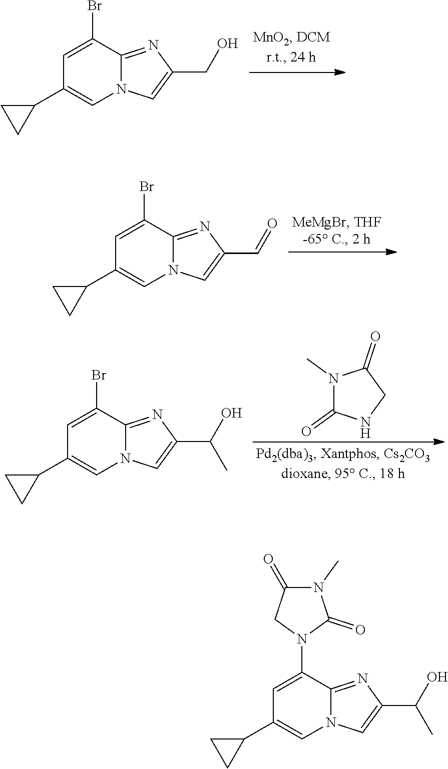

### Synthesis of 8-bromo-6-cyclopropylimidazo[1,2-a]pyridine-2-carbaldehyde

A mixture of (8-bromo-6-cyclopropylimidazo[1,2-a]pyridin-2-yl)methanol (10 g, 37.45 mmol) and MnO₂ (9.8 g, 112 mmol) was stirred at room temperature for 12 h. Second batch of MnO₂ (9.8 g, 112 mmol) was added and stirred at room temperature for another 12 h. The reaction mixture was filtered through a short silica pad and washed with EtOAc (500 mL). The filtrate was concentrated in vacuo to give 8-bromo-6-cyclopropylimidazo[1,2-a]pyridine-2-carbaldehyde (8 g, 80.8%) as a yellow solid. ESI-MS [M+H]⁺: 266.2.

### Synthesis of 1-(8-bromo-6-cyclopropylimidazo[1,2-a]pyridin-2-yl)ethan-1-ol

To a solution of 8-bromo-6-cyclopropylimidazo[1,2-a]pyridine-2-carbaldehyde (8 g, 30 mmol) in dry THF (100 mL) was added MeMgBr (30 mL, 3M solution in Et2O, 90 mmol) at −65° C. slowly and the resulting solution was stirred at −65° C. for 2 h. The reaction was quenched with saturated aqueous NH₄Cl (70 mL) at −65° C., stirred and warmed to room temperature then extracted with EtOAc (4×75 mL). The combined organic layers were washed with brine (75 mL), dried over Na₂SO₄ then concentrated in vacuo to give the crude product, which was purified with silica gel chromatography (eluent: DCM/MeOH=15/1) to give 1-(8-bromo-6-cyclopropylimidazo[1,2-a]pyridin-2-yl)ethan-1-ol (7.3 g, 86.9%) as a yellow solid. ESI-MS [M+H]⁺: 281.2.

### Synthesis of 1-(6-cyclopropyl-2-(1-hydroxyethyl)imidazo[1,2-a]pyridin-8-yl)-3-methylimidazolidine-2,4-dione

A mixture of 1-(8-bromo-6-cyclopropylimidazo[1,2-a]pyridin-2-yl)ethan-1-ol (7.3 g, 25.98 mmol), 3-methylimidazolidine-2,4-dione (8.9 g, 77.9 mmol), Pd₂(dba)₃ (2.38 g, 2.6 mmol), Xantphos (3 g, 5.2 mmol) and Cs₂CO₃ (25 g, 77 mmol) in dioxane (75 mL) was stirred at 95° C. for 18 h. The reaction mixture was cooled to room temperature, filtered through celite, washed with EtoAc (3×75 mL). The combined filtrate was washed with brine (80 mL), dried over Na₂SO₄ then concentrated in vacuo to give the crude product, which was purified with silica gel chromatography (eluent: DCM/MeOH=15/1) to give 1-(6-cyclopropyl-2-(1-hydroxyethyl)imidazo[1,2-a]pyridin-8-yl)-3-methylimidazolidine-2,4-dione (6.1 g, 75%) as a yellow solid. ESI-MS [M+H]⁺: 315.2.

### Synthesis of 3-(2-((tert-butyldimethylsilyl)oxy)ethyl)-1-(6-cyclopropyl-2-(hydroxymethyl)imidazo[1,2-a]pyridin-8-yl)imidazolidine-2,4-dione

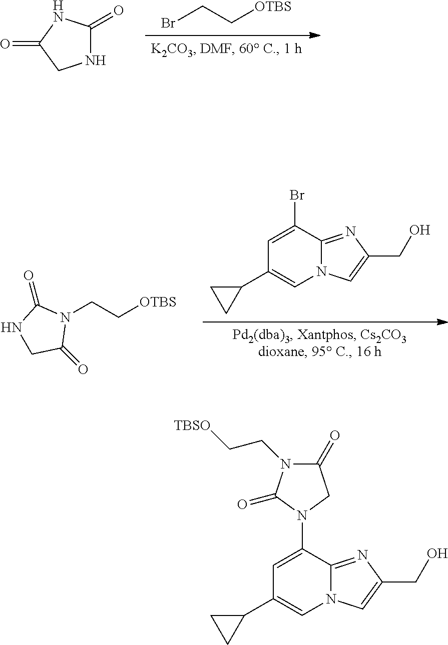

### Synthesis of 3-(2-((tert-butyldimethylsilyl)oxy)ethyl)imidazolidine-2,4-dione

To the mixture of imidazolidine-2,4-dione (5 g, 59 mmol), K₂CO₃ (20.7 g, 150 mmol) in DMF (40 mL) was added (2-bromoethoxy)(tert-butyl)dimethylsilane (9.57 g, 40 mmol). The resulting reaction mixture was stirred at 60° C. for 1 h under N₂. After cooling to room temperature, water (50 mL) was added and the mixture was extracted with EtOAc (3×30 mL). The combined organic layers were washed with brine (60 mL), dried over anhydrous Na₂SO₄ and concentrated in vacuo to give the crude product, which was purified by silica gel chromatography (eluent: MeOH/DCM=0˜1/10) to afford 3-(2-((tert-butyldimethylsilyl)oxy)ethyl)imidazolidine-2,4-dione (710 mg, 5.5%) as pale solid. ESI-MS [M+H]⁺: 259.2.

### Synthesis of 3-(2-((tert-butyldimethylsilyl)oxy)ethyl)-1-(6-cyclopropyl-2-(hydroxymethyl)imidazo[1,2-a]pyridin-8-yl)imidazolidine-2,4-dione

To a mixture of 3-(2-((tert-butyldimethylsilyl)oxy)ethyl)imidazolidine-2,4-dione (720 mg, 2.79 mmol), (8-bromo-6-cyclopropylimidazo[1,2-a]pyridin-2-yl)methanol (250 mg, 0.93 mmol), and Cs₂CO₃ (909 mg, 2.79 mmol) in dioxane (35 mL) was added Pd₂(dba)₃ (170 mg, 0.186 mmol) and Xantphos (215 mg, 0.372 mmol). The reaction mixture was stirred at 95° C. for 16 h under N₂ and then cooled to room temperature. The solution was filtered through celite and the filter cake was washed with DCM/MeOH (10/1, 50 mL). The filtrate was concentrated in vacuo to give the crude, which was purified by column chromatography (eluent: EtOAc/PE=1/100˜100/1) to afford 3-(2-((tert-butyldimethylsilyl)oxy)ethyl)-1-(6-cyclopropyl-2-(hydroxymethyl)imidazo[1,2-a]pyridin-8-yl)imidazolidine-2,4-dione (300 mg, 73%) as yellow solid. ESI-MS [M+H]⁺: 445.3.

### Synthesis of methyl 2-(3-(6-cyclopropyl-2-(hydroxymethyl)imidazo[1,2-a]pyridin-8-yl)-2,5-dioxoimidazolidin-1-yl)acetate

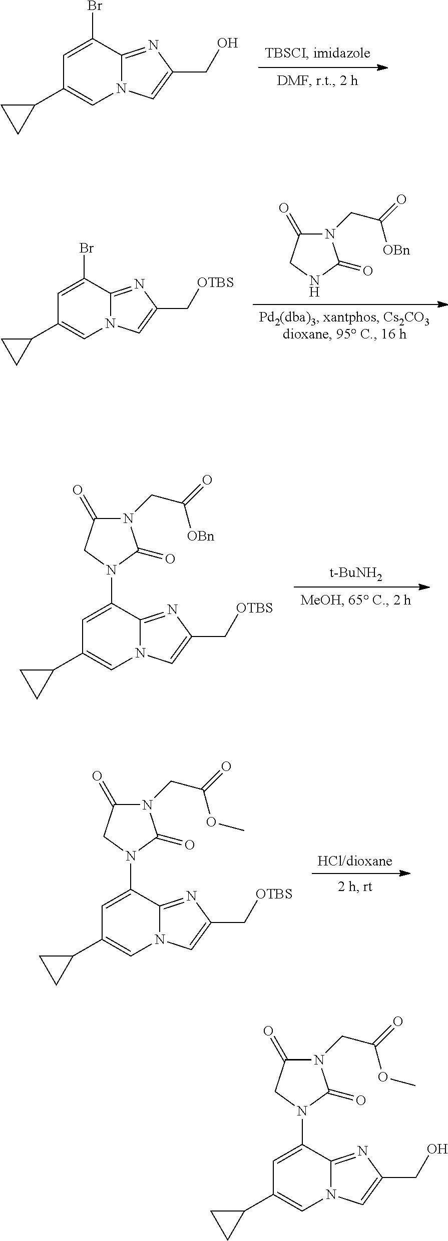

### Synthesis of 8-bromo-2-(((tert-butyldimethylsilyl)oxy)methyl)-6-cyclopropylimidazo[1,2-a]pyridine

To a solution of (8-bromo-6-cyclopropylimidazo[1,2-a]pyridin-2-yl)methanol (2 g, 7.5 mmol) in DMF (20 mL) was added tert-butylchlorodimethylsilane (1.69 g, 11.2 mmol) and imidazole (1.53 g, 22.5 mmol). The mixture was stirred at 25° C. for 2 h, then water (50 mL) was added and the mixture was extracted with EtOAc (30 mL×3). The combined organic layers were washed with brine (30 mL), dried over Na₂SO₄ and concentrated in vacuo. The resulting residue was purified by column chromatography (eluent: PE:EtAOc=1:1) to give 8-bromo-2-(((tert-butyldimethylsilyl)oxy)methyl)-6-cyclopropylimidazo[1,2-a]pyridine (2.6 g, 91%) as a purple oil. ESI-MS [M+H]⁺: 381.1.

### Synthesis of benzyl 2-(3-(2-(((tert-butyldimethylsilyl)oxy)methyl)-6-cyclopropylimidazo[1,2-a]pyridin-8-yl)-2,5-dioxoimidazolidin-1-yl)acetate

To a solution of benzyl 2-(2,5-dioxoimidazolidin-1-yl)acetate (3.1 g, 12.6 mmol) in dioxane (20 mL) was added 8-bromo-2-(((tert-butyldimethylsilyl)oxy)methyl)-6-cyclopropylimidazo[1,2-a]pyridine (1.2 g, 3.15 mmol), Pd₂(dba)₃ (577 mg, 0.63 mmol), Xantphos (729 mg, 1.26 mmol) and Cs₂CO₃ (3.0 g, 9.45 mmol). The mixture was stirred at 95° C. for 16 h under N₂, cooled to room temperature, quenched with water (50 mL) and extracted with EtOAc (3×50 ml). The combined organics were washed with brine (50 mL), dried over Na₂SO₄ and concentrated. The crude residue was purified by column chromatography (eluent: PE:EtAOc=1:1) to give benzyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (1 g, 58%) as a yellow oil. ESI-MS [M+H]⁺: 549.2.

### Synthesis of methyl 2-(3-(2-(((tert-butyldimethylsilyl)oxy)methyl)-6-cyclopropylimidazo[1,2-a]pyridin-8-yl)-2,5-dioxoimidazolidin-1-yl)acetate

To a mixture of benzyl 2-(3-(2-(((tert-butoxycarbonyl)(methyl)amino)methyl)-6-cyclopropylimidazo[1,2-a]pyridin-8-yl)-2,5-dioxoimidazolidin-1-yl)acetate (1 g, 1.82 mmol) in MeOH (15 mL) was added t-BuN₃ (2.7 g, 36.4 mmol). The mixture was stirred at 65° C. for 2 h under N₂ then cooled to room temperature and filtered. The filtrate was concentrated to give the crude product which was purified by column chromatography (eluent: DCM/MeOH=20/1) to give methyl 2-(3-(2-(((tert-butyldimethylsilyl)oxy)methyl)-6-cyclopropylimidazo[1,2-a]pyridin-8-yl)-2,5-dioxoimidazolidin-1-yl)acetate (800 mg, 93%) as a yellow solid. ESI-MS [M+H]⁺: 473.2.

### Synthesis of methyl 2-(3-(6-cyclopropyl-2-(hydroxymethyl)imidazo[1,2-a]pyridin-8-yl)-2,5-dioxoimidazolidin-1-yl)acetate

To a solution of methyl 2-(3-(2-(((tert-butyldimethylsilyl)oxy)methyl)-6-cyclopropylimidazo[1,2-a]pyridin-8-yl)-2,5-dioxoimidazolidin-1-yl)acetate (500 mg, 1.05 mmol) in MeOH (3 mL) was added HCl/dioxane (5.0 mL, 20 mmol) at 0° C. and the mixture was stirred at RT for 2 h. Then the mixture was quenched with water (50 mL) and extracted with EtOAc (3×50 ml). The combined organics were washed with brine (50 mL), dried over Na₂SO₄ and concentrated in vacuo. The residue was purified by preparative TLC (eluent: DCM/MeOH=10/1) to give methyl 2-(3-(6-cyclopropyl-2-(hydroxymethyl)imidazo[1,2-a]pyridin-8-yl)-2,5-dioxoimidazolidin-1-yl)acetate (370 mg, 98%) as a yellow solid. ESI-MS [M+H]⁺: 359.1.

### Synthesis of 3-(6-cyclopropyl-2-(hydroxymethyl)imidazo[1,2-a]pyridin-8-yl)-3-azabicyclo[3.1.0]hexan-2-one

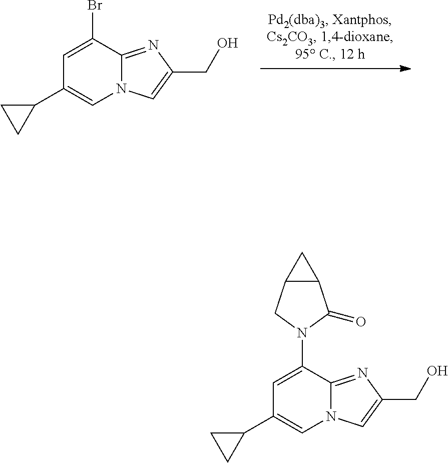 [SMILES: OCc1cn2cc(C3CC3)cc(Br)c2n1 ; "Pd2(dba)3, Xantphos,Cs2CO3, 1,4-dioxane,95° C., 12 h" ; O=C1C2CC2CN1c1cc(C2CC2)cn2cc(CO)nc12]

To a mixture of (8-bromo-6-cyclopropylimidazo[1,2-a]pyridin-2-yl)methanol (300 mg, 1.12 mmol), 3-azabicyclo[3.1.0]hexan-2-one (217.3 mg, 2.24 mmol) and Cs₂CO₃ (1.1 g, 3.36 mmol) in 1,4-dioxane (5 mL) was added Pd₂(dba)₃ (102.5 mg, 0.112 mmol) and Xantphos (129.6 mg, 0.224 mmol). The reaction mixture was stirred at 95° C. for 12 h under N₂ and cooled to room temperature. The solution was filtered through Celite® and the filter cake was washed with DCM/MeOH (10/1, 100 mL). The filtrate was concentrated in vacuo to give the crude, which was purified by preparative TLC (DCM/MeOH=20/1) to give 3-(6-cyclopropyl-2-(hydroxymethyl)imidazo[1,2-a]pyridin-8-yl)-3-azabicyclo[3.1.0]hexan-2-one (250 mg, 79%) as a yellow solid. ESI-MS [M+H]+: 284.2.

### Synthesis of 1-(7-cyclopropyl-2-(hydroxymethyl)imidazo[1,2-a]pyridin-5-yl)-3-methylimidazolidine-2,4-dione

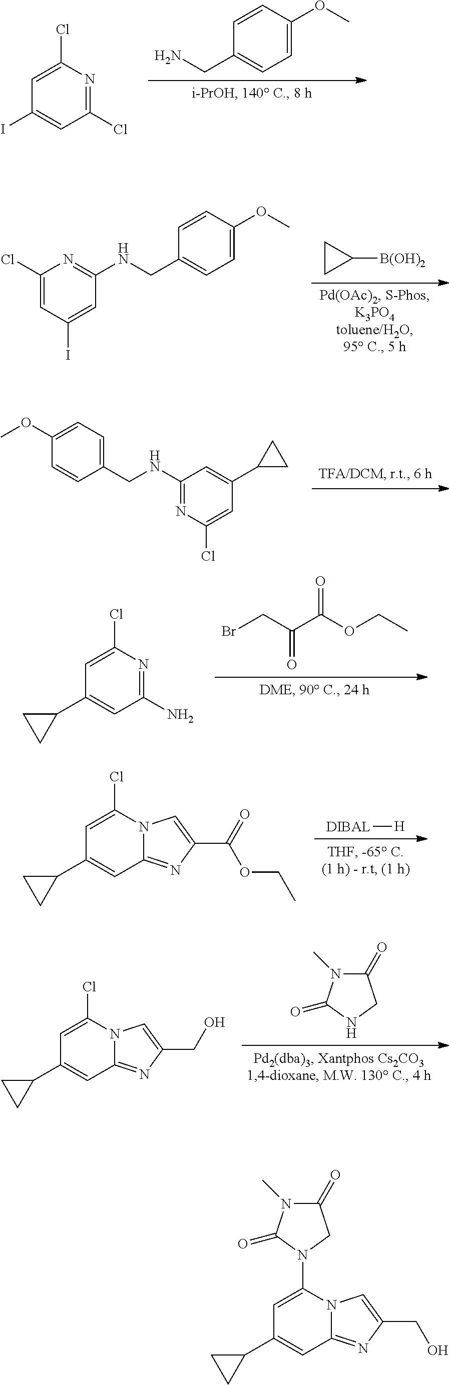

### Synthesis of 6-chloro-4-iodo-N-(4-methoxybenzyl)pyridin-2-amine

A mixture of 2,6-dichloro-4-iodopyridine (5.0 g, 18.26 mmol) and (4-methoxyphenyl)methanamine (12.5 g, 91.12 mmol) in i-PrOH (100 mL) was stirred in a sealed tube at 140° C. for 8 h. The resulting mixture was concentrated in vacuo to give the crude, which was purified by silica gel chromatography (eluent: PE/EtOAc=20/1) to give 6-chloro-4-iodo-N-(4-methoxybenzyl)pyridin-2-amine (2.2 g, 32%) as a white solid. ESI-MS: [M+H]⁺: 374.9.

### Synthesis of 6-chloro-4-cyclopropyl-N-(4-methoxybenzyl)pyridin-2-amine

To a mixture of 6-chloro-4-iodo-N-(4-methoxybenzyl)pyridin-2-amine (2.5 g, 6.67 mmol), cyclopropylboronic acid (575 mg, 6.69 mmol), and K₃PO₄ (4.25 g, 20.02 mmol) in H₂O (6 mL) and toluene (60 mL) was added Pd(OAc)₂ (299 mg, 1.33 mmol) and S-Phos (1.1 g, 2.68 mmol). The reaction mixture was stirred at 95° C. for 5 h under N₂. The reaction mixture was filtered through celite and the filter cake was washed with EtOAc (50 mL). The filtrate was concentrated in vacuo to give the crude, which was purified by silica gel chromatography (eluent: PE/EtOAc=10/1) to give 6-chloro-4-cyclopropyl-N-(4-methoxybenzyl)pyridin-2-amine (1.4 g, 73%) as a yellow solid. ESI-MS: [M+H]⁺: 289.1.

### Synthesis of 6-chloro-4-cyclopropylpyridin-2-amine

To a solution of 6-chloro-4-cyclopropyl-N-(4-methoxybenzyl)pyridin-2-amine (2.5 g, 8.66 mmol) in DCM (10 mL) was added TFA (2 mL). The reaction mixture was stirred at room temperature for 6 h. After cooling to 0° C., the mixture was adjusted to pH 8 by adding saturated NaHCO₃ solution. The resulting mixture was then extracted with DCM (300 mL×3). The combined organic layers were washed with brine (50 mL), dried over anhydrous Na₂SO₄ and concentrated in vacuo to give the crude, which was purified by silica gel chromatography (eluent: PE/EA=2/1) to give 6-chloro-4-cyclopropylpyridin-2-amine (1.4 g, 95.9%) as a yellow solid. ESI-MS: [M+H]⁺, 289.1.

### Synthesis of ethyl 5-chloro-7-cyclopropylimidazo[1,2-a]pyridine-2-carboxylate

To a solution of 6-chloro-4-cyclopropylpyridin-2-amine (1.4 g, 8.30 mmol) in DME (30 mL) was added ethyl 3-bromo-2-oxopropanoate (1.9 g, 9.74 mmol). The reaction mixture was stirred at 90° C. for 24 h under N₂. After cooling to room temperature, water (100 mL) was added and then extracted with DCM (400 mL×3). The combined organic layers were washed with brine (100 mL), dried over anhydrous Na₂SO₄, and concentrated in vacuo to give the crude, which was purified by silica gel chromatography (eluent: EA/PE=3/2) to give 5-chloro-7-cyclopropylimidazo[1,2-a]pyridine-2-carboxylate (740 mg, 34%) as a yellow solid. ESI-MS: [M+H]⁺, 265.1.

### Synthesis of (5-chloro-7-cyclopropylimidazo[1,2-a]pyridin-2-yl)methanol

To a solution of ethyl 5-chloro-7-cyclopropylimidazo[1,2-a]pyridine-2-carboxylate (2.5 g, 9.47 mmol) in THF (50 mL) was added DIBAL-H (1M solution in hexanes, 18.94 mL, 18.94 mmol) dropwise at −65° C. The resulting reaction was stirred at −65° C. for 1 h under N₂, then warmed to room temperature for another 1 h. The reaction was quenched with H₂O (5 mL) at 0° C., followed by NaOH (5 mL, 15% aqueous solution) and H₂O (20 mL). The reaction mixture was warmed to room temperature and stirred for another 15 min then extracted with EtOAc (50 mL×3). The combined organic layers were washed with brine (80 mL), dried over anhydrous Na₂SO₄, and concentrated in vacuo to give the crude, which was purified by silica chromatography (eluent: DCM/MeOH=25/1) to give (5-chloro-7-cyclopropylimidazo[1,2-a]pyridin-2-yl)methanol (1.48 g, 70%) as a yellow solid. ESI-MS: [M+H]⁺, 223.0

### Synthesis of 1-(7-cyclopropyl-2-(hydroxymethyl)imidazo[1,2-a]pyridin-5-yl)-3-methylimidazolidine-2,4-dione

To a solution of (5-chloro-7-cyclopropylimidazo[1,2-a]pyridin-2-yl)methanol (230 mg, 1.04 mmol), 3-methylimidazolidine-2,4-dione (948 mg, 8.32 mmol) and Cs₂CO₃ (1.02 g, 3.12 mmol) in 1,4-dioxane (10 mL) was added Pd₂(dba)₃ (192 mg, 0.21 mmol) and XantPhos (243 mg, 0.42 mmol). The reaction mixture was stirred in a sealed tube under microwave at 130° C. for 4 h. The reaction mixture was filtered through celite and the filter cake was washed with DCM/MeOH (10/1, 100 mL). The filtrate was concentrated in vacuo to give the crude, which was purified by silica gel chromatography (eluent: DCM/MeOH=25/1) to give 1-(7-cyclopropyl-2-(hydroxymethyl)imidazo[1,2-a]pyridin-5-yl)-3-methylimidazolidine-2,4-dione (300 mg, 96%) as a yellow solid. ESI-MS: [M+H]⁺, 301.1

### Synthesis of (6-cyclopropyl-8-(4-methylpiperazin-1-yl)imidazo[1,2-a]pyridin-2-yl)methanol

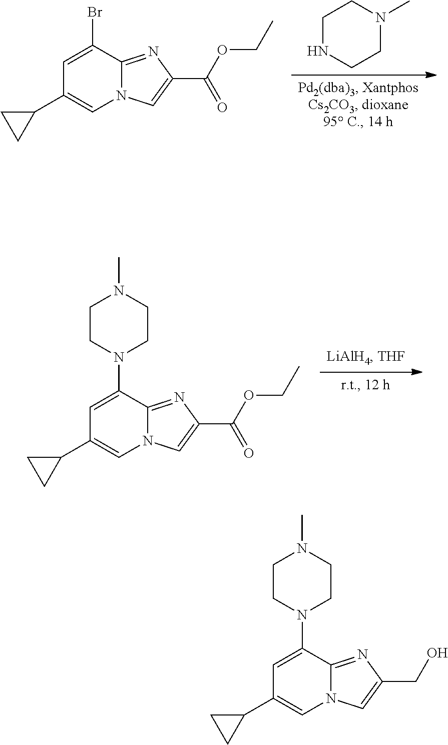

### Synthesis of 2-(((2-bromopyridin-4-yl)oxy)methyl)-5-cyclopropyl-7-(4-methylpiperazin-1-yl)pyrazolo[1,5-a]pyridine

To a mixture of ethyl 8-bromo-6-cyclopropylimidazo[1,2-a]pyridine-2-carboxylate (500 mg, 1.6 mmol), 1-methylpiperazine (320 mg, 3.2 mmol) and Cs₂CO₃ (1.56 g, 4.8 mmol) in 1,4-dioxane (5 mL) was added Pd₂(dba)₃ (146.5 mg, 0.16 mmol) and Xantphos (185.2 mg, 0.32 mmol). The resulting mixture was stirred at 95° C. for 14h under N₂. The reaction mixture was filtered through Celite® and the filter cake was washed with DCM/MeOH (10/1, 10 mL). The filtrate was concentrated in vacuo to give the crude, which was purified by preparative TLC (DCM/MeOH=20/1) to give ethyl 6-cyclopropyl-8-(4-methylpiperazin-1-yl)imidazo[1,2-a]pyridine-2-carboxylate (300 mg, 57%) as a white solid. ESI-MS [M+H]+: 329.2.

### Synthesis of (6-cyclopropyl-8-(4-methylpiperazin-1-yl)imidazo[1,2-a]pyridin-2-yl)methanol

To a mixture of 6-cyclopropyl-8-(4-methylpiperazin-1-yl)imidazo[1,2-a]pyridine-2-carboxylate (300 mg, 0.91 mmol) in THF (10 mL) was added LiAlH₄ (103.4 mg, 2.73 mmol) at 0° C. After stirring at room temperature for 12 h, the reaction was quenched with saturated aq·NH₄Cl (25 mL) then extracted with EtOAc (3×30 mL). The combined organic layers were washed with brine, dried over Na₂SO₄, filtered and concentrated in vacuo to give the crude, which was purified by preparative TLC (DCM/MeOH=60/1) to give (6-cyclopropyl-8-(4-methylpiperazin-1-yl)imidazo[1,2-a]pyridin-2-yl)methanol (180 mg, 69%) as a white solid. ESI-MS [M+H]+: 287.2.

### Synthesis of (8-(benzyloxy)-6-cyclopropylimidazo[1,2-a]pyridin-2-yl)methanamine

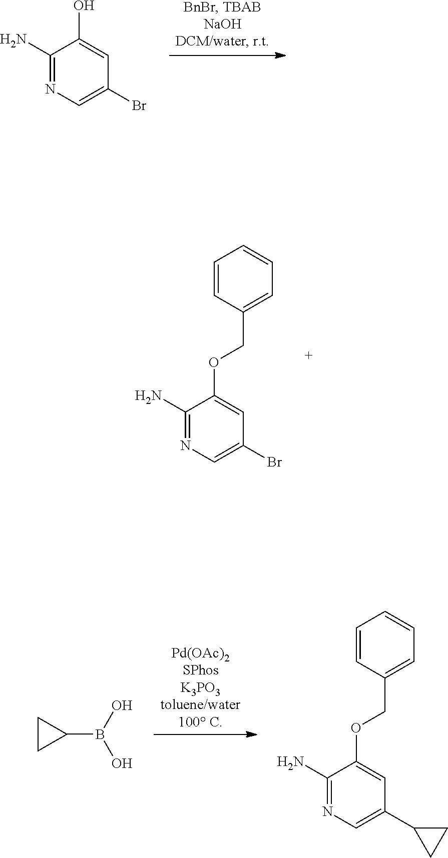 [SMILES: Nc1ncc(Br)cc1O ; "BnBr, TBABNaOHDCM/water, r.t." ; Nc1ncc(Br)cc1OCc1ccccc1 ; OB(O)C1CC1 ; "Pd(OAc)2SPhosK3PO3toluene/water100° C." ; Nc1ncc(C2CC2)cc1OCc1ccccc1]

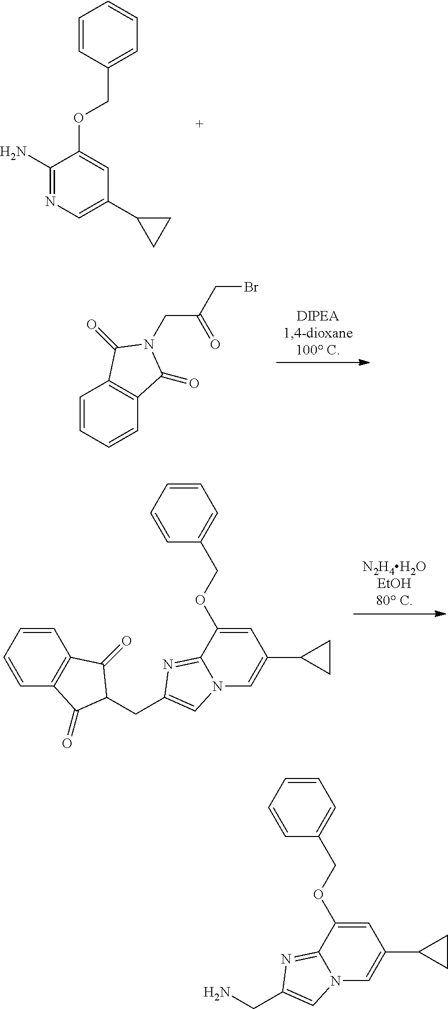 [SMILES: Nc1ncc(C2CC2)cc1OCc1ccccc1 ; O=C(CBr)CN1C(=O)c2ccccc2C1=O ; "DIPEA1,4-dioxane100° C." ; O=C1c2ccccc2C(=O)C1Cc1cn2cc(C3CC3)cc(OCc3ccccc3)c2n1 ; "N2H4·H2OEtOH80° C." ; NCc1cn2cc(C3CC3)cc(OCc3ccccc3)c2n1]

### Synthesis of 3-(benzyloxy)-5-bromopyridin-2-amine

2-Amino-5-bromopyridin-3-ol (13 g, 67 mmol) was added portionwise to a vigorously stirred mixture of NaOH (15 g, 380 mmol) in water (24 mL) and DCM (40 mL). TBAB (0.36 g, 1.1 mmol) was added and the mixture was stirred at room temperature for 15 min. A solution of benzyl bromide (13 g, 74 mmol) in DCM (40 mL) was added and the mixture was stirred at room temperature for 18 h. Water (100 mL) was added and the mixture was extracted with DCM (3×100 mL). The combined organic layers were dried (MgSO₄), filtered and concentrated in vacuo. The residue was purified by column chromatography on silica gel, eluting with 20-80% EtOAc in cyclohexane to give the title compound (13 g, 67%) as a yellow solid.

ESI-MS (M+H)+: 281.1, 279.1 ¹H NMR (400 MHz, DMSO) δ 7.60-7.59 (m, 1H), 7.53-7.50 (m, 2H), 7.44-7.34 (m, 3H), 7.29 (d, J=2.0 Hz, 1H), 5.96 (s, 2H), 5.17 (s, 2H).

### Synthesis of 3-(benzyloxy)-5-cyclopropylpyridin-2-amine

A mixture of 3-(benzyloxy)-5-bromopyridin-2-amine (13 g, 45 mmol), cyclopropylboronic acid (5.8 g, 67 mmol), Pd(OAc)₂ (0.50 g, 2.2 mmol), SPhos (1.8 g, 4.5 mmol) and K₃PO₄ (33 g, 160 mmol) in toluene (130 mL) and water (13 mL) was degassed with N₂ then stirred at 100° C. under a N₂ atmosphere for 18 h. The mixture was cooled and the organic layer was decanted and filtered through Celite®. The filtrate was diluted with EtOAc and the organic layer further decanted and repeatedly filtered through Celite®. The combined filtrates were concentrated in vacuo and the residue was purified by column chromatography on silica gel, eluting with 20-100% EtOAc in cyclohexane to give the title compound (8.7 g, 89%) as a yellow solid.

ESI-MS (M+H)+: 241.3, ¹H NMR (400 MHz, DMSO) δ 7.51-7.49 (m, 2H), 7.42-7.31 (m, 4H), 6.77 (d, J=1.9 Hz, 1H), 5.41 (s, 2H), 5.12 (s, 2H), 1.80-1.73 (m, 1H), 0.85-0.79 (m, 2H), 0.58-0.53 (m, 2H).

### Synthesis of 2-((8-(benzyloxy)-6-cyclopropylimidazo[1,2-a]pyridin-2-yl)methyl)isoindoline-1,3-dione

A mixture of 3-(benzyloxy)-5-cyclopropylpyridin-2-amine (4.1 g, 17 mmol), 2-(3-bromo-2-oxopropyl)isoindoline-1,3-dione (5.2 g, 18.6 mmol) and DIPEA (3.2 mL, 19 mmol) in 1,4-dioxane (340 mL) was stirred at 100° C. for 18 h then concentrated in vacuo. The residue was dissolved in DCM (250 mL) and washed with water (150 mL), then a solution of NaHCO₃ (sat·aq·, 150 mL), then brine (sat·aq·, 150 mL) and filtered through a phase separator then concentrated in vacuo. The residue was purified by column chromatography on silica gel, eluting with 0-100% EtOAc in cyclohexane to give the title compound (2.4 g, 34%) as a green solid.

ESI-MS (M+H)+: 424.4 ¹H NMR (400 MHz, DMSO) δ 7.94-7.86 (m, 5H), 7.71 (s, 1H), 7.49-7.46 (m, 2H), 7.43-7.35 (m, 3H), 6.44 (d, J=1.1 Hz, 1H), 5.25 (s, 2H), 4.84 (s, 2H), 1.91-1.83 (m, 1H), 0.92-0.86 (m, 2H), 0.68-0.63 (m, 2H).

### Synthesis of (8-(benzyloxy)-6-cyclopropylimidazo[1,2-a]pyridin-2-yl)methanamine

A mixture of 2-((8-(benzyloxy)-6-cyclopropylimidazo[1,2-a]pyridin-2-yl)methyl)isoindoline-1,3-dione (0.50 g, 1.2 mmol) and hydrazine monohydrate (0.74 mL, 12 mmol) in EtOH (12 mL) was stirred at 80° C. for 1 h. The mixture was cooled to room temperature, loaded onto an SCX cartridge and washed with a mixture of DCM and MeOH (1:1). The product was eluted with a mixture of DCM and 7 N NH₃ in MeOH (1:1) to give the title compound (450 mg, quantitative) as a yellow gum.

ESI-MS (M+H)+: 294.2 ¹H NMR (400 MHz, DMSO) δ 7.95 (s, 1H), 7.63 (s, 1H), 7.51 (d, J=6.9 Hz, 2H), 7.46-7.37 (m, 3H), 6.44 (s, 1H), 5.26 (s, 2H), 3.77 (s, 2H), 1.94-1.86 (m, 1H), 0.93-0.87 (m, 2H), 0.71-0.66 (m, 2H). NH₂ not observed

### Synthesis of 3-((2-(aminomethyl)-6-cyclopropylimidazo[1,2-a]pyridin-8-yl)oxy)-1-methylpyrrolidin-2-one

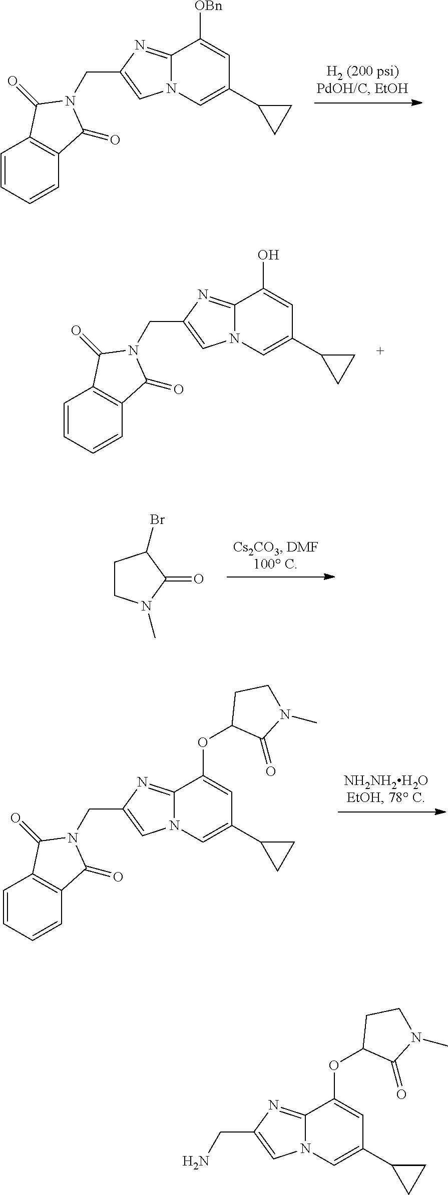

### Synthesis of 2-((6-cyclopropyl-8-hydroxyimidazo[1,2-a]pyridin-2-yl)methyl)isoindoline-1,3-dione

2-((8-(benzyloxy)-6-cyclopropylimidazo[1,2-a]pyridin-2-yl)methyl)isoindoline-1,3-dione (1.90 g, 4.49 mmol) and palladium hydroxide (10 mol % on carbon, 1.9 g, 1.35 mmol) were suspended in ethanol (90 mL) and degassed with N₂ for 5 min. The reaction was then placed under H₂ (200 psi) and the reaction stirred at room temperature for 18 h. The reaction was filtered through Celite® which was then washed with MeOH (50 mL). The reaction was concentrated in vacuo. The residue was purified by silica gel chromatography, eluting with a gradient of 0-20% MeOH in DCM to give the title compound (790 mg, 53%). ESI-MS (M+H)+: 334.1, ¹H NMR (400 MHz, DMSO) δ 7.98-7.90 (m, 4H), 7.84 (d, J=1.0 Hz, 1H), 7.74 (s, 1H), 6.26-6.23 (m, 1H), 4.90 (s, 2H), 1.91-1.76 (m, 1H), 0.94-0.88 (m, 2H), 0.65-0.60 (m, 2H).

### Synthesis of 2-((6-cyclopropyl-8-((1-methyl-2-oxopyrrolidin-3-yl)oxy)imidazo[1,2-a]pyridin-2-yl)methyl)isoindoline-1,3-dione

2-((6-cyclopropyl-8-hydroxyimidazo[1,2-a]pyridin-2-yl)methyl)isoindoline-1,3-dione (200 mg, 0.60 mmol), 3-bromo-1-methylpyrrolidin-2-one (117 mg, 0.66 mmol) and Cs₂CO₃ (215 mg, 0.66 mmol) were suspended in DMF (3.0 mL) and the reaction was heated to 100° C. for 30 min. The reaction was cooled to room temperature and diluted with EtOAc (10 mL) and NaHCO₃ (sat·aq·, 15 mL), the layers were separated and the aqueous layer was further extracted with EtOAc (2×30 mL). The combined organics were dried over MgSO₄ then concentrated in vacuo. The residue was purified by silica gel chromatography, eluting with a gradient of 0-20% (7N NH₃ in MeOH) in DCM to give the title compound (200 mg, 78%). ESI-MS (M+H)+: 431.3, ¹H NMR (400 MHz, CDCl₃) δ 7.88-7.85 (m, 2H), 7.71 (dd, J=3.0, 5.6 Hz, 2H), 7.48 (s, 1H), 7.39 (s, 1H), 6.76-6.73 (m, 1H), 5.30 (s, 2H), 5.26 (dd, J=5.4, 8.0 Hz, 1H), 3.62-3.55 (m, 1H), 3.40-3.32 (m, 1H), 2.61-2.38 (m, 2H), 1.87-1.78 (m, 2H), 1.47-1.43 (m, 1H), 0.92-0.86 (m, 2H), 0.66-0.61 (m, 2H).

### Synthesis of 3-((2-(aminomethyl)-6-cyclopropylimidazo[1,2-a]pyridin-8-yl)oxy)-1-methylpyrrolidin-2-one

Using a similar procedure to that for 3-(2-(aminomethyl)-6-cyclopropylimidazo[1,2-a]pyridin-8-yl)oxetan-3-ol, 3-((2-(aminomethyl)-6-cyclopropylimidazo[1,2-a]pyridin-8-yl)oxy)-1-methylpyrrolidin-2-one (125 mg, 89%) was synthesised from 2-((6-cyclopropyl-8-((1-methyl-2-oxopyrrolidin-3-yl)oxy)imidazo[1,2-a]pyridin-2-yl)methyl)isoindoline-1,3-dione (200 mg, 0.46 mmol) using hydrazine hydrate. ESI-MS (M+H)+: 301.2, ¹H NMR (400 MHz, MeOD) δ 7.88 (s, 1H), 7.65 (s, 1H), 6.76 (s, 1H), 5.30 (dd, J=6.2, 7.8 Hz, 1H), 3.93 (s, 2H), 3.66-3.59 (m, 1H), 3.56-3.47 (m, 1H), 2.96 (s, 3H), 2.72-2.60 (m, 1H), 2.32-2.23 (m, 1H), 1.99-1.92 (m, 1H), 1.01-0.92 (m, 2H), 0.79-0.74 (m, 2H).

The compounds in Table A12 were synthesized using a similar method to 3-((2-(aminomethyl)-6-cyclopropylimidazo[1,2-a]pyridin-8-yl)oxy)-1-methylpyrrolidin-2-one, starting from 2-((6-cyclopropyl-8-hydroxyimidazo[1,2-a]pyridin-2-yl)methyl)isoindoline-1,3-dione and the appropriate alkyl halide:

TABLE A12

| Compound | Alkyl halide | Analytical data |
|---|---|---|
| 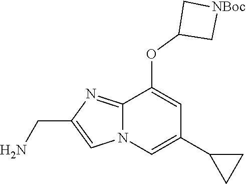 [SMILES: CC(C)(C)OC(=O)N1CC(Oc2cc(C3CC3)cn3cc(CN)nc23)C1] | tert-butyl 3- iodoazetidine-1- carboxylate | 1H NMR (400 MHz, DMSO) δ 8.02 (s, 1H), 7.70 (s, 1H), 6.18 (d, J = 1.3 Hz, 1H), 5.30-5.23 (m, 1H), 4.39 (dd, J = 5.6, 8.1 Hz, 2H), 3.91 (dd, J = 2.9, 9.5 Hz, 2H), 1.98-1.90 (m, 1H), 1.45 (s, 9H), 0.95 (ddd, J = 4.4, 6.4, 8.4 Hz, 2H), 0.76-0.71 (m, 2H) |
| tert-butyl 3-((2-(aminomethyl)-6- | | |
| cyclopropylimidazo[1,2-a]pyridin-8- | | |
| yl)oxy)azetidine-1-carboxylate | | |

### Synthesis of (6-cyclopropyl-8-(2-methoxypyridin-3-yl)imidazo[1,2-a]pyridin-2-yl)methanamine

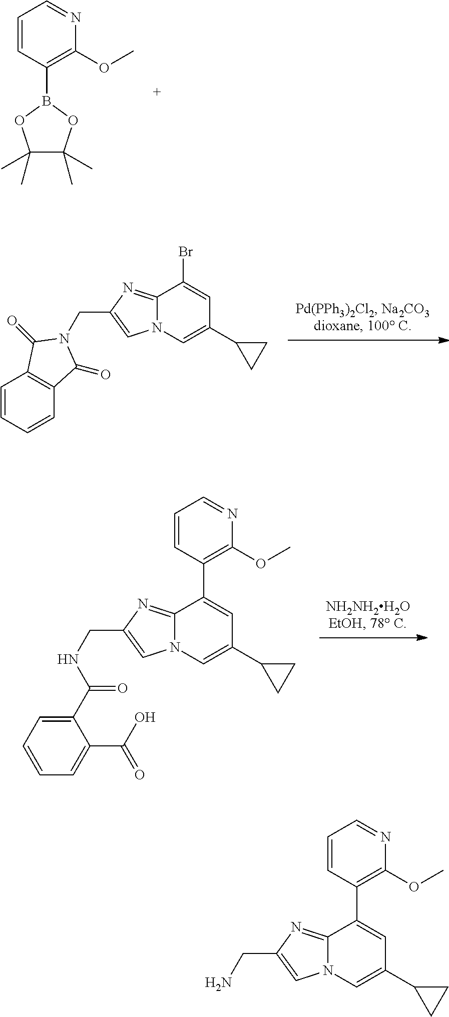

### Synthesis of 2-(((6-cyclopropyl-8-(2-methoxypyridin-3-yl)imidazo[1,2-a]pyridin-2-yl)methyl)carbamoyl)benzoic acid

2-((8-bromo-6-cyclopropylimidazo[1,2-a]pyridin-2-yl)methyl)isoindoline-1,3-dione (500 mg, 1.26 mmol), 2-methoxy-3-(4,4,5,5-tetramethyl-1,3,2-dioxaborolan-2-yl)pyridine (326 mg, 1.39 mmol) and Na₂CO₃ (1M aq·, 3.8 mL, 3.8 mmol) were suspended in 1,4-dioxane (5.0 mL) and degassed with N₂ for 5 min. Pd(PPh₃)Cl₂ (89 mg, 0.126 mmol) was then added and the reaction was heated to 100° C. under a N₂ environment for 30 min. The reaction was cooled to room temperature and filtered through Celite®, which was then washed with DCM:MeOH 2:1 (10 mL). The reaction was concentrated in vacuo. The residue was purified by silica gel chromatography, eluting with a gradient of 0-100% (7N NH₃ in MeOH) in DCM to give the title compound (550 mg, 98%).

ESI-MS (M+H)+: 443.4, ¹H NMR (400 MHz, CDCl₃) δ 8.27-8.26 (1H, m), 8.06-8.06 (2H, m), 7.74-7.73 (2H, m), 7.56-7.54 (2H, m), 7.17-7.17 (2H, m), 6.98 (1H, s), 6.82-6.82 (1H, m), 3.81 (2H, s), 3.47 (3H, s), 1.85-1.76 (1H, m), 0.94-0.87 (2H, m), 0.62-0.58 (2H, m)

### Synthesis of (6-cyclopropyl-8-(2-methoxypyridin-3-yl)imidazo[1,2-a]pyridin-2-yl)methanamine

Using a similar procedure to that for 3-(2-(aminomethyl)-6-cyclopropylimidazo[1,2-a]pyridin-8-yl)oxetan-3-ol, (6-cyclopropyl-8-(2-methoxypyridin-3-yl)imidazo[1,2-a]pyridin-2-yl)methanamine (370 mg, 99%) was synthesised from 2-(((6-cyclopropyl-8-(2-methoxypyridin-3-yl)imidazo[1,2-a]pyridin-2-yl)methyl)carbamoyl)benzoic acid (550 mg, 1.26 mmol) using hydrazine hydrate.

ESI-MS (M+H)+: 295.3, ¹H NMR (400 MHz, MeOD) δ 8.14-8.11 (m, 2H), 7.76 (dd, J=2.0, 7.3 Hz, 1H), 7.67 (s, 1H), 7.01-6.97 (m, 2H), 3.88 (s, 2H), 3.79 (s, 3H), 1.93-1.85 (m, 1H), 0.93-0.87 (m, 2H), 0.67-0.62 (m, 2H).

### Synthesis of ethyl 3-(2-(aminomethyl)-6-cyclopropylimidazo[1,2-a]pyridin-8-yl)propanoate

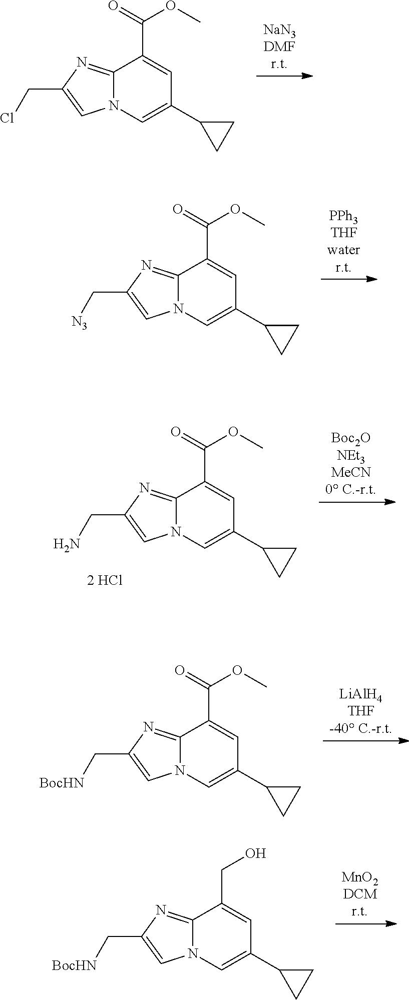

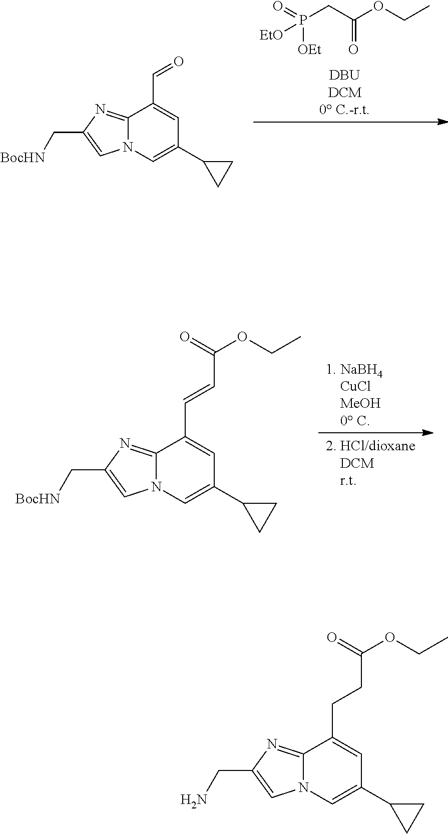

### Synthesis of methyl 2-(azidomethyl)-6-cyclopropylimidazo[1,2-a]pyridine-8-carboxylate

Sodium azide (1.1 g, 17 mmol) was added to a mixture of methyl 2-(chloromethyl)-6-cyclopropylimidazo[1,2-a]pyridine-8-carboxylate hydrochloride (3.4 g, 11 mmol) and NEt₃ (2.6 g, 26 mmol) in DMF (25 mL). The mixture was stirred at room temperature under a nitrogen atmosphere for 72 h. The mixture was poured into EtOAc (330 mL) and washed with water (330 mL), brine (sat·aq·, 330 mL), water (330 mL), dried over MgSO₄, filtered and concentrated in vacuo to give the title compound (2.7 g, 77%) as a brown oil. ESI-MS [M+H]+: 272.2, ¹H NMR (400 MHz, DMSO) δ 8.62 (dd, J=0.6, 1.8 Hz, 1H), 7.96 (m, 1H), 7.64 (d, J=1.9 Hz, 1H), 4.55 (s, 2H), 3.89 (s, 3H), 2.06-1.99 (m, 1H), 0.99-0.93 (m, 2H), 0.75-0.70 (m, 2H).

### Synthesis of methyl 2-(aminomethyl)-6-cyclopropylimidazo[1,2-a]pyridine-8-carboxylate dihydrochloride

A mixture of methyl 2-(azidomethyl)-6-cyclopropylimidazo[1,2-a]pyridine-8-carboxylate (2.7 g, 10 mmol) and triphenylphosphine (5.3, 20 mmol) in THF (45 mL) and water (5.0 mL) was stirred at room temperature for 18 h. The mixture was concentrated in vacuo then dissolved in DCM and treated with HCl (4.0 M in 1,4-dioxane). The precipitate was collected by filtration and washed with Et₂O to give the title compound (2.4 g, 76%) as a cream solid. ESI-MS [M+H]+: 246.2, ¹H NMR (400 MHz, DMSO) δ 8.98 (s, 1H), 8.62 (br s, 3H), 8.25 (s, 1H), 8.12 (s, 1H), 4.36 (s, 2H), 4.00 (s, 3H), 2.18-2.14 (m, 1H), 1.10-1.04 (m, 2H), 0.86-0.82 (m, 2H).

### Synthesis of methyl 2-(((tert-butoxycarbonyl)amino)methyl)-6-cyclopropylimidazo[1,2-a]pyridine-8-carboxylate

Di-tert-butyl dicarbonate (1.7 g, 7.6 mmol) was added to a mixture of methyl 2-(aminomethyl)-6-cyclopropylimidazo[1,2-a]pyridine-8-carboxylate dihydrochloride (2.4 g, 7.6 mmol) and NEt₃ (5.3 mL, 38 mmol) in MeCN (25 mL) 0° C. The mixture allowed to warm to room temperature and stirred for 18 h under a nitrogen atmosphere. The mixture was diluted with NaHCO₃ (sat·aq·, 50 mL) and water (50 mL) then extracted with DCM (100 mL×3). The combined organic layers were dried over MgSO₄, filtered and concentrated in vacuo to the title compound (2.2 g, 82%) as a yellow gum. ESI-MS [M+H]+: 346.2, ¹H NMR (400 MHz, DMSO) δ 8.59 (s, 1H), 7.70 (s, 1H), 7.36-7.34 (m, 1H), 4.23 (d, J=5.6 Hz, 2H), 3.88 (s, 3H), 2.04-1.95 (m, 1H), 1.40 (s, 9H), 0.98-0.91 (m, 2H), 0.73-0.67 (m, 2H), exchangeable NH not observed.

### Synthesis of tert-butyl ((6-cyclopropyl-8-(hydroxymethyl)imidazo[1,2-a]pyridin-2-yl)methyl)carbamate

LiAlH₄ (1.0 M in THF, 6.3 mL, 6.3 mmol) was added dropwise to a solution of methyl 2-(((tert-butoxycarbonyl)amino)methyl)-6-cyclopropylimidazo[1,2-a]pyridine-8-carboxylate (2.2 g, 6.3 mmol) in THF (41 mL) at −40° C. under a nitrogen atmosphere. The mixture was stirred at −40° C. for 30 min then warmed to 0° C. and stirred for 30 min then warmed to room temperature and stirred for 30 min. The mixture was cooled to 0° C. and further LiAlH₄ (1.0 M in THF, 3.2 mL, 3.2 mmol) was added. The mixture was warmed to room temperature and stirred for 45 min. The mixture was cooled to 0° C. and water (1.5 mL) and NaOH (20% aq·, 0.35 mL) were added. The mixture was warmed to room temperature and stirred for 15 min. Et₂O (10 mL) was added and the mixture was filtered through Celite® with THF and concentrated in vacuo. The residue was redissolved in THF (21 mL) and cooled to −40° C. under a nitrogen atmosphere. LiAlH₄ (1.0 M in THF, 2.8 mL, 2.8 mmol) was added dropwise and the mixture was stirred at −40° C. for 1.5 h. Further LiAlH₄ (1.0 M in THF, 2.8 mL, 2.8 mmol) was added and the mixture was stirred at −40° C. for a further 30 min. The mixture was warmed to room temperature and stirred for 2 h. The mixture was cooled to 0° C. and water (0.21 mL), NaOH (20% aq, 0.16 mL) and water (0.66 mL) were added slowly. The mixture was warmed to room temperature and stirred for 15 min. MgSO₄ was added and the mixture was filtered through Celite® with THF and concentrated in vacuo to give the title compound (1.5 g, 75%) as a yellow gum. ESI-MS [M+H]+: 318.2, ¹H NMR (400 MHz, DMSO) δ 8.22 (s, 1H), 7.58 (s, 1H), 7.29-7.26 (m, 1H), 6.97-6.95 (m, 1H), 5.30 (t, J=5.7 Hz, 1H), 4.75 (d, J=5.3 Hz, 2H), 4.19 (d, J=5.9 Hz, 2H), 1.97-1.89 (m, 1H), 1.40 (s, 9H), 0.95-0.89 (m, 2H), 0.68-0.63 (m, 2H).

### Synthesis of tert-butyl ((6-cyclopropyl-8-formylimidazo[1,2-a]pyridin-2-yl)methyl)carbamate

To a solution of tert-butyl ((6-cyclopropyl-8-(hydroxymethyl)imidazo[1,2-a]pyridin-2-yl)methyl)carbamate (400 mg, 1.3 mmol) in DCM (15 mL) was added MnO₂ (1.1 g, 13 mmol). The mixture was stirred at room temperature for 2 h under a nitrogen atmosphere. The mixture was filtered through Celite® with DCM then THF (×3) and concentrated in vacuo to give the title compound (210 mg, 32%) as a yellow oil. ESI-MS [M+H]+: 316.2, ¹H NMR (400 MHz, DMSO) δ 10.48 (s, 1H), 8.70 (s, 1H), 7.75 (s, 1H), 7.42-7.36 (m, 1H), 6.87 (s, 1H), 4.27 (d, J=5.9 Hz, 2H), 2.08-1.99 (m, 1H), 1.41 (s, 9H), 1.01-0.94 (m, 2H), 0.77-0.71 (m, 2H).

### Synthesis of ethyl (E)-3-(2-(((tert-butoxycarbonyl)amino)methyl)-6-cyclopropylimidazo[1,2-a]pyridin-8-yl)acrylate

DBU (85 μl, 0.57 mmol) was added slowly to a solution of tert-butyl ((6-cyclopropyl-8-formylimidazo[1,2-a]pyridin-2-yl)methyl)carbamate (180 mg, 0.57 mmol) and triethyl phosphonoacetate (140 mg, 0.628 mmol) in DCM (10 mL) at 0° C. under a nitrogen atmosphere. The mixture was stirred at room temperature for 72 h. The mixture was diluted with water (20 mL) and brine (sat·aq·, 30 mL) and extracted with DCM (3×50 mL). The combined organic layers were passed through a phase separator and concentrated in vacuo. The residue was purified by column chromatography on silica gel, eluting with 1-5% NH₃ in MeOH in DCM to give the title compound (270 mg, quant.) as a yellow oil which was used directly in the next step without further purification. ESI-MS [M+H]+: 386.3.

### Synthesis of ethyl 3-(2-(((tert-butoxycarbonyl)amino)methyl)-6-cyclopropylimidazo[1,2-a]pyridin-8-yl)propanoate

NaBH₄ (240 mg, 6.2 mmol) was added to a mixture of ethyl (E)-3-(2-(((tert-butoxycarbonyl)amino)methyl)-6-cyclopropylimidazo[1,2-a]pyridin-8-yl)acrylate (240 mg, 0.62 mmol) and CuCl (62 mg, 0.62 mmol) in MeOH (10 mL) in three portions at 0° C. under a nitrogen atmosphere. The mixture was stirred at 0° C. for 3 h. The mixture was diluted with NaHCO₃ (sat·aq·, 5.0 mL), water (50 mL) and NaHCO₃ (sat·aq·, 45 mL) and extracted with EtOAc (3×75 mL). The combined organic layers were dried over MgSO₄, filtered and concentrated in vacuo to give the title compound (280 mg, quant.) as a yellow oil which was used directly in the next step without further purification. ESI-MS [M+H]+: 388.3.

### Synthesis of ethyl 3-(2-(aminomethyl)-6-cyclopropylimidazo[1,2-a]pyridin-8-yl)propanoate

HCl (4.0 M in 1,4-dioxane, 0.9 mL, 3.6 mmol) was added to a solution of ethyl 3-(2-(((tert-butoxycarbonyl)amino)methyl)-6-cyclopropylimidazo[1,2-a]pyridin-8-yl)propanoate (280 mg, 0.72 mmol) in DCM (2 mL). The mixture was stirred at room temperature for 90 min then concentrated in vacuo to give the title compound (250 mg, quant.) as a yellow oil which was used directly in the next step.

ESI-MS [M+H]+: 288.3.

### Synthesis of (6-cyclopropyl-8-(methylsulfonyl)imidazo[1,2-a]pyridin-2-yl)methanol

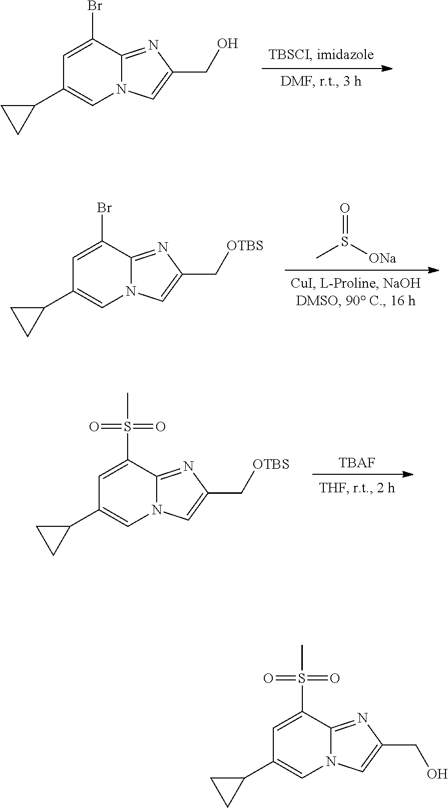

### Synthesis of 8-bromo-2-(((tert-butyldimethylsilyl)oxy)methyl)-6-cyclopropylimidazo[1,2-a]pyridine

To a mixture of (8-bromo-6-cyclopropylimidazo[1,2-a]pyridin-2-yl)methanol (0.20 g, 0.75 mmol), imidazole (0.77 g, 11 mmol) in DMF (10 mL) was added TBSCl (0.85 g, 5.6 mmol). The mixture was stirred at room temperature for 3 h under N₂. The reaction was quenched with water (20 mL) and extracted with EtOAc (3×50 mL). The combined organic layers were washed with saturated brine solution (30 mL), dried over Na₂SO₄, and concentrated in vacuo to give the crude, which was purified by silica gel chromatography (eluent: PE/EtOAc=20/1˜5/1) to give 8-bromo-2-(((tert-butyldimethylsilyl) oxy)methyl)-6-cyclopropylimidazo[1,2-a]pyridine (0.15 g, 52%) as a yellow oil. ESI-MS [M+H]+: 383.1

### Synthesis of 2-(((tert-butyldimethylsilyl)oxy)methyl)-6-cyclopropyl-8-(methylsulfonyl)imidazo[1,2-a]pyridine

To a mixture of 8-bromo-2-(((tert-butyldimethylsilyl)oxy)methyl)-6-cyclopropylimidazo[1,2-a]pyridine (0.96 g, 2.5 mmol) in DMSO (25 mL) were added sodium methanesulfinate (2.6 g, 25 mmol), NaOH (0.10 g, 2.5 mmol), L-Proline (0.29 g, 2.5 mmol) and CuI (0.49 g, 2.6 mmol). The mixture was stirred at 90° C. for 16 h. After cooling to room temperature, the reaction was quenched with water (30 mL) and extracted with EtOAc (3×50 mL). The combine organic layers were washed with saturated brine solution (60 mL), dried over Na₂SO₄, and concentrated in vacuo to give the crude, which was purified by column chromatography on silica gel (eluent: PE/EtOAc=10/1˜3/1) to give 2-(((tert-butyldimethylsilyl)oxy)methyl)-6-cyclopropyl-8-(methylsulfonyl)-imidazo[1,2-a]pyridine (0.59 g, 62%) as a yellow solid. ESI-MS [M+H]+: 381.1

### Synthesis of (6-cyclopropyl-8-(methylsulfonyl)imidazo[1,2-a]pyridin-2-yl)methanol

To a mixture 2-(((tert-butyldimethylsilyl)oxy)methyl)-6-cyclopropyl-8-(methylsulfonyl)imidazo[1,2-a]pyridine (0.30 g, 0.79 mmol) in THF (10 mL) was added TBAF (1.6 mL, 1.6 mmol). After stirring at room temperature for 2 h, the mixture was concentrated in vacuo to give the crude (6-cyclopropyl-8-(methylsulfonyl)imidazo[1,2-a]pyridin-2-yl)methanol (0.10 g, 48%) as a white solid.

ESI-MS [M+H]+: 267.2

### Synthesis of (6-cyclopropyl-8-(methylsulfonyl)imidazo[1,2-a]pyridin-2-yl)methanamine

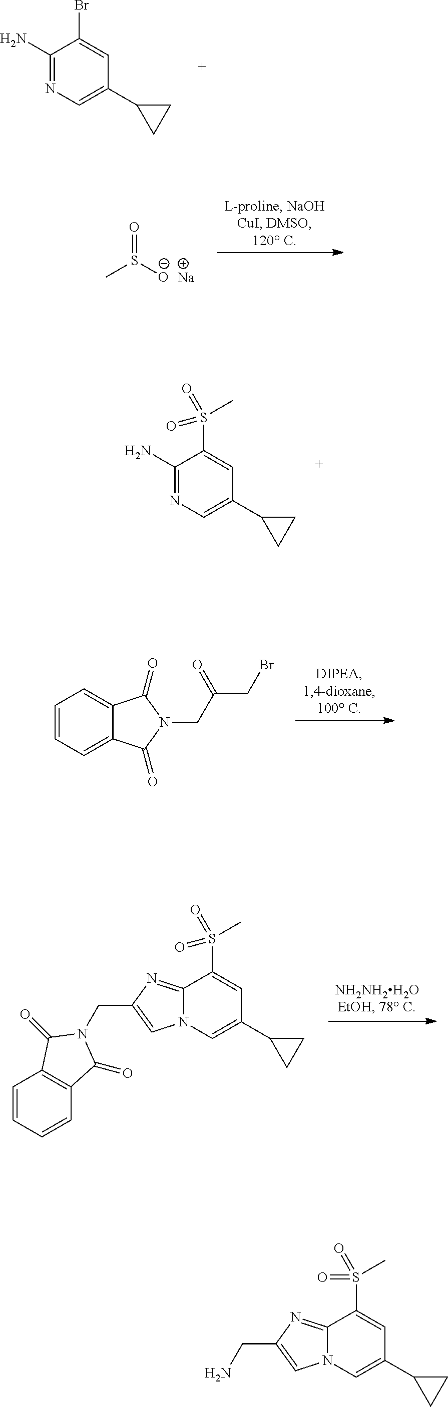

### Synthesis of 5-cyclopropyl-3-(methylsulfonyl)pyridin-2-amine

3-bromo-5-cyclopropylpyridin-2-amine (250 mg, 1.17 mmol), sodium methanesulfinate (299 mg, 2.93 mmol), L-proline (135 mg, 1.17 mmol), NaOH (47 mg, 1.17 mmol) and CuI (223 mg, 1.17 mmol) were suspended in DMSO (1.5 mL), the reaction was degassed with N₂ for 5 min. The reaction was then heated at 120° C. for 18 h. The reaction was cooled to room temperature then filtered through Celite®. The Celite® was then washed with 10% MeOH in DCM (20 mL). The washings were concentrated in vacuo, and the residue was purified by silica gel chromatography, eluting with a gradient of 25-100% EtOAc in cyclohexane to give the title compound (145 mg, 58%). ¹H NMR (400 MHz, CDCl₃) δ 8.23-8.18 (m, 1H), 7.67 (dd, J=4.0, 4.0 Hz, 1H), 5.76 (s, 2H), 3.07-3.06 (m, 3H), 1.88-1.79 (m, 1H), 1.00-0.83 (m, 2H), 0.68-0.53 (m, 2H).

### Synthesis of 2-((6-cyclopropyl-8-(methylsulfonyl)imidazo[1,2-a]pyridin-2-yl)methyl)isoindoline-1,3-dione

DIPEA (0.24 mL, 1.37 mmol) was added to a stirred solution of 5-cyclopropyl-3-(methylsulfonyl)pyridin-2-amine (145 mg, 0.686 mmol) and 2-(3-bromo-2-oxopropyl)isoindoline-1,3-dione (194 mg, 0.686 mmol) in 1,4-dioxane (7.0 mL). The reaction was heated at 100° C. for 18 h. The reaction was cooled to room temperature and the reaction was concentrated in vacuo. The residue was dissolved in EtOAc (100 mL) and washed with NaHCO₃ (sat·aq·, 2×40 mL) and brine (50 mL). The combined organics were dried over MgSO₄ and concentrated in vacuo. The residue was purified by silica gel chromatography, eluting with a gradient of 20-100% EtOAc in cyclohexane to give the title compound (100 mg, 37%). ¹H NMR (400 MHz, CDCl₃) δ 8.00 (d, J=1.0 Hz, 1H), 7.89-7.86 (m, 2H), 7.74 (dd, J=2.9, 5.4 Hz, 2H), 7.68-7.65 (m, 1H), 7.55 (s, 1H), 5.11 (s, 2H), 3.51 (s, 2H), 1.96-1.84 (m, 1H), 1.04-0.94 (m, 2H), 0.72-0.63 (m, 2H).

### Synthesis of (6-cyclopropyl-8-(methylsulfonyl)imidazo[1,2-a]pyridin-2-yl)methanamine

Using a similar procedure to that for 3-(2-(aminomethyl)-6-cyclopropylimidazo[1,2-a]pyridin-8-yl)oxetan-3-ol, (6-cyclopropyl-8-(methylsulfonyl)imidazo[1,2-a]pyridin-2-yl)methanamine (67 mg) was synthesised from 2-((6-cyclopropyl-8-(methylsulfonyl)imidazo[1,2-a]pyridin-2-yl)methyl)isoindoline-1,3-dione (100 mg, 0.253 mmol) using hydrazine hydrate. The product was used without further purification.

### Synthesis of (R)-1-(6-cyclopropyl-8-(methylsulfonyl)imidazo[1,2-a]pyridin-2-yl)ethan-1-amine

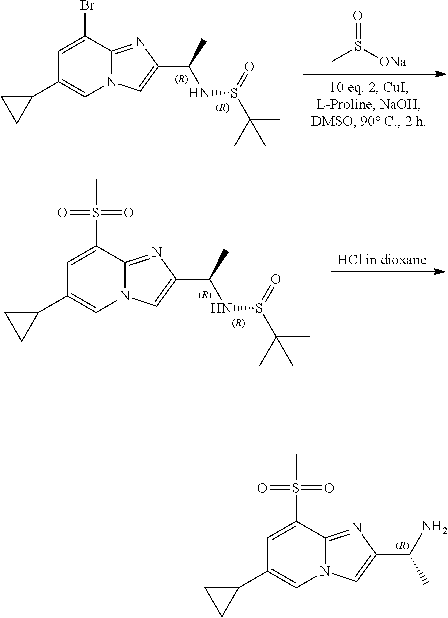

### Synthesis of (R)—N—((R)-1-(6-cyclopropyl-8-(methylsulfonyl)imidazo[1,2-a]pyridin-2-yl)ethyl)-2-methylpropane-2-sulfinamide

To a mixture of (R)—N—((R)-1-(8-bromo-6-cyclopropylimidazo[1,2-a]pyridin-2-yl)ethyl)-2-methylpropane-2-sulfinamide (200 mg, 0.52 mmol), sodium methanesulfinate (530 mg, 5.2 mmol), L-Proline (60 mg, 0.52 mmol) and NaOH (21 mg, 0.52 mmol) in DMSO (6 mL) was added CuI (99 mg, 0.52 mmol). The reaction mixture was stirred at 90° C. for 2 h under N₂ and cooled to room temperature. The solution was filtered through celite and the filter cake was washed with DCM/MeOH (10/1, 30 mL). The filtrate was concentrated in vacuo to give the crude, which was purified by preparative TLC (eluent: PE/EtOAc=20/1) to give (R)—N—((R)-1-(6-cyclopropyl-8-(methylsulfonyl)imidazo[1,2-a]pyridin-2-yl)ethyl)-2-methylpropane-2-sulfinamide (150 mg, 50%) as a yellow oil. ESI-MS [M+H]+: 384.2.

### Synthesis of (R)-1-(6-cyclopropyl-8-(methylsulfonyl)imidazo[1,2-a]pyridin-2-yl)ethan-1-amine

To a solution of (R)—N—((R)-1-(6-cyclopropyl-8-(methylsulfonyl)imidazo[1,2-a]pyridin-2-yl)ethyl)-2-methylpropane-2-sulfinamide (150 mg, 0.39 mmol) in dioxane (5 mL) was added HCl (4M solution in dioxane, 5 mL). The resulting mixture was stirred at room temperature for 1 h. and was concentrated in vacuo to give (R)-1-(6-cyclopropyl-8-(methylsulfonyl)imidazo[1,2-a]pyridin-2-yl)ethan-1-amine as the hydrochloric acid salt (100 mg, crude) as a yellow oil which was used in next step without purification. ESI-MS [M+H]+: 280.1.

### Synthesis of 1-(6-cyclopropyl-8-(methylsulfonyl)imidazo[1,2-a]pyridin-2-yl)-N-methylmethanamine

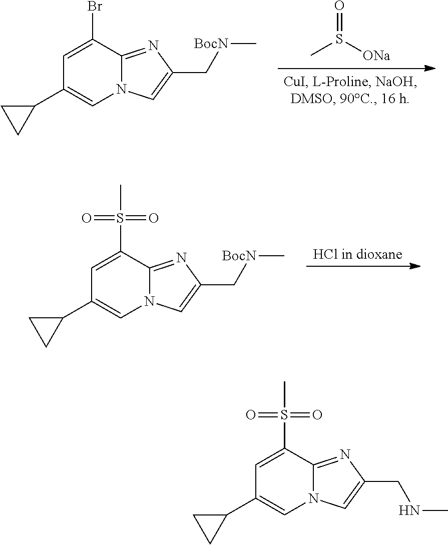

### Synthesis of tert-butyl ((6-cyclopropyl-8-(methylsulfonyl)imidazo[1,2-a]pyridin-2-yl)methyl)(methyl)carbamate

To a mixture of tert-butyl ((8-bromo-6-cyclopropylimidazo[1,2-a]pyridin-2-yl)methyl)(methyl)carbamate (200 mg, 0.53 mmol), sodium methanesulfinate (540 mg, 5.3 mmol), L-Proline (61 mg, 0.53 mmol) and NaOH (21 mg, 0.53 mmol) in DMSO (6 mL) was added CuI (101 mg, 0.53 mmol). The reaction mixture was stirred at 90° C. for 16 h under N₂ and then cooled to room temperature. The solution was filtered through celite and the filter cake was washed with DCM/MeOH (10/1, 30 mL). The filtrate was concentrated in vacuo to give the crude product, which was purified by preparative TLC (eluent: PE/EtOAc=1/1) to give (tert-butyl ((6-cyclopropyl-8-(methylsulfonyl)imidazo[1,2-a]pyridin-2-yl)methyl)(methyl)carbamate (160 mg, 80%) as a yellow solid. ESI-MS [M+H]+: 380.2.

### Synthesis of 1-(6-cyclopropyl-8-(methylsulfonyl)imidazo[1,2-a]pyridin-2-yl)-N-methylmethanamine

To a solution of tert-butyl ((6-cyclopropyl-8-(methylsulfonyl)imidazo[1,2-a]pyridin-2-yl)methyl)(methyl)carbamate (160 mg, 0.42 mmol) in dioxane (5 mL) was added HCl (4M solution in dioxane, 5 mL). The resulting mixture was stirred at room temperature for 1 h and then concentrated in vacuo to give 1-(6-cyclopropyl-8-(methylsulfonyl)imidazo[1,2-a]pyridin-2-yl)-N-methylmethanamine as the hydrochloric acid salt (110 mg, crude) as a yellow solid which was used in next step without purification. ESI-MS [M+H]+: 280.1.

### Synthesis of 1-(8-bromo-6-methylimidazo[1,2-a]pyridin-2-yl)ethan-1-ol

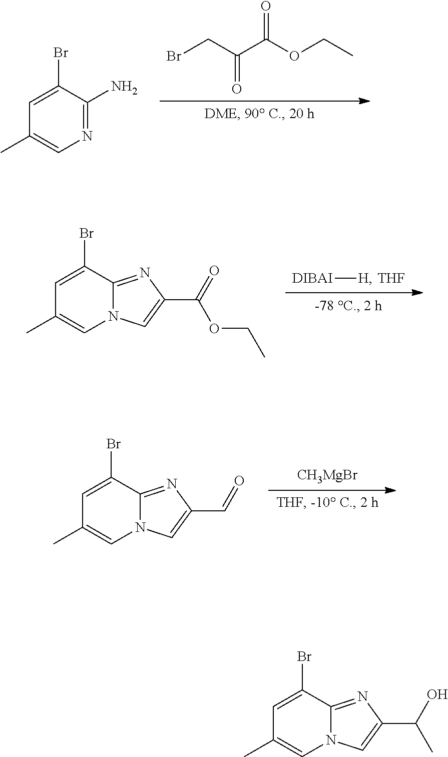

### Synthesis of ethyl 8-bromo-6-methylimidazo[1,2-a]pyridine-2-carboxylate

To a mixture of 3-bromo-5-methylpyridin-2-amine (5.0 g, 27 mmol) and ethyl 3-bromo-2-oxopropanoate (6.3 g, 32 mmol) in DME (60 mL) was stirred at 90° C. for 20 h. The reaction mixture was cooled to room temperature and was quenched with water (50 mL), extracted with EtOAc (50 mL×3). The combined organic layers were concentrated in vacuo to give the crude, which was purified by column chromatography (eluent: EtOAc/PE=0-100%) to afford ethyl 8-bromo-6-methylimidazo[1,2-a]pyridine-2-carboxylate (3.5 g, 46%) as yellow solid. ESI-MS [M+H]+: 283.0.

### Synthesis of 8-bromo-6-methylimidazo[1,2-a]pyridine-2-carbaldehyde

To a solution of ethyl 8-bromo-6-methylimidazo[1,2-a]pyridine-2-carboxylate (3.5 g, 12.4 mmol) in THF (50 mL) was added DIBAL-H (37 mL, 37 mmol, 1M in hexane) at −78° C. The reaction mixture was stirred at −78° C. for 2 h. The reaction was quenched at −65° C. by MeOH (30 mL) and 15% NaOH (aq·, 30 mL). The mixture was then allowed to room temperature, water (40 mL) was added and extracted with EtOAc (100 mL×3). The combined organic layers were washed with brine (50 mL), dried over Na₂SO₄ and concentrated in vacuo. The residue was purified by column chromatography (eluent: EtOAc/PE=0-80%) to afford 8-bromo-6-methylimidazo[1,2-a]pyridine-2-carbaldehyde (2.4 g, 81%) as yellow solid. ESI-MS [M+H]+: 239.0.

### Synthesis of 1-(8-bromo-6-methylimidazo[1,2-a]pyridin-2-yl)ethan-1-ol

To a solution of 8-bromo-6-methylimidazo[1,2-a]pyridine-2-carbaldehyde (1.2 g, 5.0 mmol) in THF (25 mL) was added CH₃MgBr (5.0 mL, 15 mmol, 3M in ether) at −10° C. and the reaction mixture was stirred at this temperature for 2 h. The reaction was quenched with NH₄Cl (sat, a.q., 50 mL) at −10° C. and extracted with EtOAc (30 mL×3). The combined organic layers were washed with brine (50 mL), dried over Na₂SO₄ and concentrated in vacuo. The residue was purified by column chromatography (eluent: EtOAc/PE=0-80%) to afford 1-(8-bromo-6-methylimidazo[1,2-a]pyridin-2-yl)ethan-1-ol (1.0 g, 79%) as a yellow solid. ESI-MS [M+H]+: 255.0.

### Synthesis of (R)-1-(2-(1-aminoethyl)-6-methylimidazo[1,2-a]pyridin-8-yl)-3-methylimidazolidine-2,4-dione

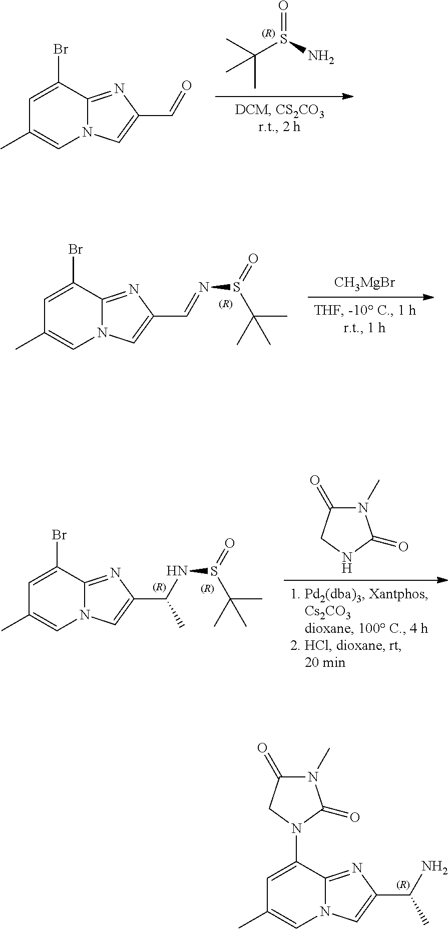

### Synthesis of (R,E)-N-((8-bromo-6-methylimidazo[1,2-a]pyridin-2-yl)methylene)-2-methylpropane-2-sulfinamide

To a mixture of 8-bromo-6-methylimidazo[1,2-a]pyridine-2-carbaldehyde (12.0 g, 50 mmol) in DCM (200 mL) was added (R)-2-methylpropane-2-sulfinamide (9.1 g, 75 mmol) and Cs₂CO₃ (32.7 g, 100 mmol). The resulting mixture was stirred at room temperature for 4 h. The reaction mixture was poured into water (200 mL) and extracted with DCM (3×200 mL). The combined organics were washed with brine (200 mL), dried over Na₂SO₄ and concentrated in vacuo. The residue was purified by silica gel chromatography (eluent: EtOAc/PE=0˜50%) to give (R,E)-N-((8-bromo-6-methylimidazo[1,2-a]pyridin-2-yl)methylene)-2-methylpropane-2-sulfinamide (10 g, yield 58%) as a yellow oil. ESI-MS [M+H]+: 342.0

### Synthesis of (R)—N—((R)-1-(8-bromo-6-methylimidazo[1,2-a]pyridin-2-yl)ethyl)-2-methylpropane-2-sulfinamide

To a mixture of (R,E)-N-((8-bromo-6-methylimidazo[1,2-a]pyridin-2-yl)methylene)-2-methylpropane-2-sulfinamide (400 mg, 1.17 mmol) in THF (10 mL) was added methylmagnesium bromide (1.17 mL, 3 M in THF, 3.50 mmol) at −10° C. The resulting mixture was stirred at −10° C. for 1 h and then room temperature for 1 h. The reaction mixture was quenched with saturated aq·NH₄Cl (50 mL) and extracted with EtOAc (3×30 mL). The combined organics were washed with brine (30 mL), dried over Na₂SO₄ and concentrated in vacuo. The residue was purified by silica gel chromatography (eluent: EtOAc/PE=0˜40%) to give (R)—N—((R)-1-(8-bromo-6-methylimidazo[1,2-a]pyridin-2-yl)ethyl)-2-methylpropane-2-sulfinamide (260 mg, yield 62%) as a yellow oil. ESI-MS [M+H]+: 358.0

### Synthesis of (R)-2-methyl-N—((R)-1-(6-methyl-8-(3-methyl-2,4-dioxoimidazolidin-1-yl)imidazo[1,2-a]pyridin-2-yl)ethyl)propane-2-sulfinamide

A mixture of (R)—N—((R)-1-(8-bromo-6-methylimidazo[1,2-a]pyridin-2-yl)ethyl)-2-methylpropane-2-sulfinamide (260 mg, 0.73 mmol), 3-methylimidazolidine-2,4-dione (400 mg, 3.51 mmol), Pd₂(dba)₃ (134 mg, 0.15 mmol), XantPhos (169 mg, 0.29 mmol) and Cs₂CO₃ (713 mg, 2.19 mmol) in 1,4-dioxane (6 mL) was stirred at 100° C. for 4 h under N₂. The reaction mixture was cooled to room temperature, then filtered through Celite® and the filter cake was washed with DCM/MeOH (10/1, 50 mL). The filtrate was concentrated in vacuo to give the crude, which was purified by silica gel chromatography (eluent: DCM/MeOH=0˜5%) to give (R)-2-methyl-N—((R)-1-(6-methyl-8-(3-methyl-2,4-dioxoimidazolidin-1-yl)imidazo[1,2-a]pyridin-2-yl)ethyl)propane-2-sulfinamide (230 mg, 81%) as a yellow solid. ESI-MS [M+H]+: 392.2

### Synthesis of (R)-1-(2-(1-aminoethyl)-6-methylimidazo[1,2-a]pyridin-8-yl)-3-methylimidazolidine-2,4-dione

A mixture of (R)-2-methyl-N—((R)-1-(6-methyl-8-(3-methyl-2,4-dioxoimidazolidin-1-yl)imidazo[1,2-a]pyridin-2-yl)ethyl)propane-2-sulfinamide (230 mg, 0.59 mmol) in HCl/dioxane (2 mL) was stirred at room temperature for 20 min. The reaction mixture was poured into NH₃ in methanol (10 mL) and concentrated in vacuo to give the crude, which was purified by silica gel chromatography (eluent: DCM/MeOH=0˜10%) to get (R)-1-(2-(1-aminoethyl)-6-methylimidazo[1,2-a]pyridin-8-yl)-3-methylimidazolidine-2,4-dione (150 mg, yield 88%) as a white solid. ESI-MS [M+H]+: 288.1

### Synthesis of 3-(2-((R)-1-aminoethyl)-6-methylimidazo[1,2-a]pyridin-8-yl)-3-azabicyclo[3.1.0]hexan-2-one

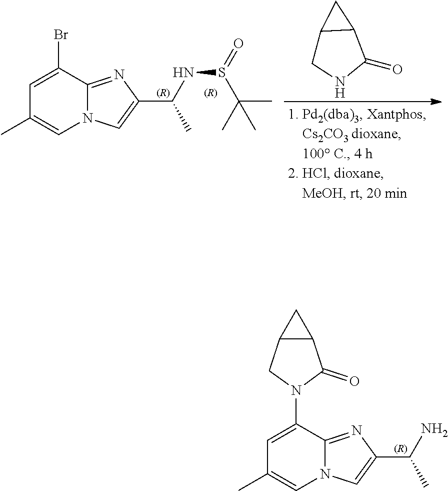

Using a similar procedure to that for (R)-1-(2-(1-aminoethyl)-6-methylimidazo[1,2-a]pyridin-8-yl)-3-methylimidazolidine-2,4-dione, 3-(2-((R)-1-aminoethyl)-6-methylimidazo[1,2-a]pyridin-8-yl)-3-azabicyclo[3.1.0]hexan-2-one hydrochloric acid salt (140 mg) was synthesised from (R)—N—((R)-1-(8-bromo-6-methylimidazo[1,2-a]pyridin-2-yl)ethyl)-2-methylpropane-2-sulfinamide (250 mg, 0.7 mmol) and 3-azabicyclo[3.1.0]hexan-2-one (135.8 mg, 1.4 mmol). ESI-MS [M+H]+: 271.2

### Synthesis of (R)-1-(2-(1-aminoethyl)-6-ethylimidazo[1,2-a]pyridin-8-yl)-3-methylimidazolidine-2,4-dione

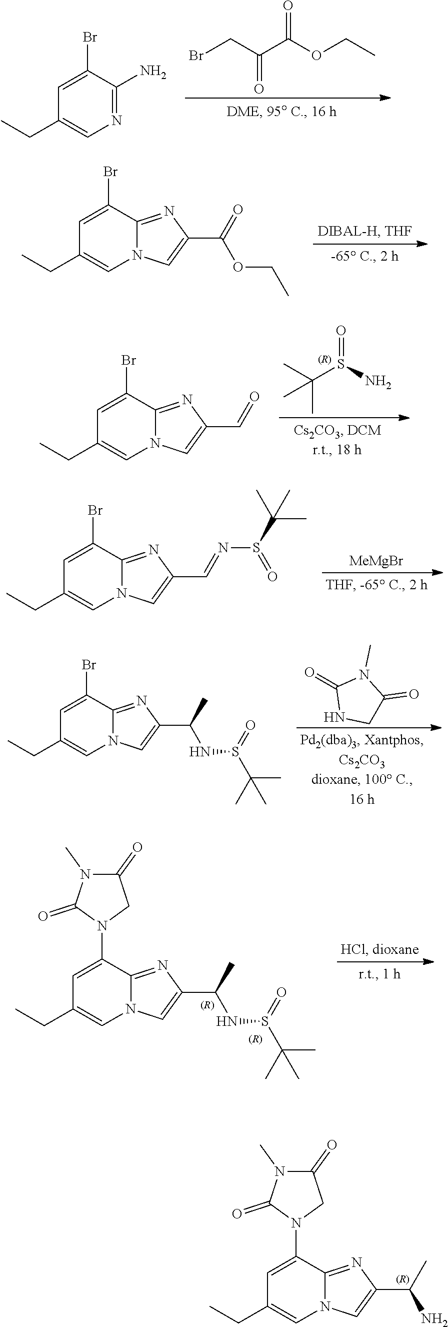

Using a similar procedure to that for (R)-1-(2-(1-aminoethyl)-6-methylimidazo[1,2-a]pyridin-8-yl)-3-methylimidazolidine-2,4-dione, (R)-1-(2-(1-aminoethyl)-6-ethylimidazo[1,2-a]pyridin-8-yl)-3-methylimidazolidine-2,4-dione (0.50 g (0.50 g) was synthesised from 3-bromo-5-ethylpyridin-2-amine. ESI-MS [M+H]+: 302.1

### Synthesis of 1-(2-(aminomethyl)-6-ethylimidazo[1,2-a]pyridin-8-yl)-3-methylimidazolidine-2,4-dione

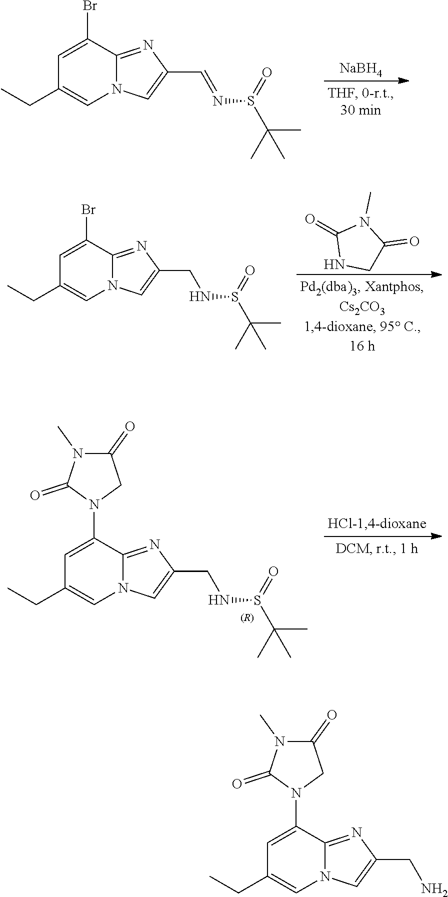

### Synthesis of (R)—N-((8-bromo-6-ethylimidazo[1,2-a]pyridin-2-yl)methyl)-2-methylpropane-2-sulfinamide

NaBH₄ (88 mg, 2.3 mmol) was added to a solution of (R,E)-N-((8-bromo-6-ethylimidazo[1,2-a]pyridin-2-yl)methylene)-2-methylpropane-2-sulfinamide (200 mg, 0.56 mmol) in THF (8 mL) at 0° C. and the mixture was stirred at room temperature for 0.5 h. The reaction was quenched with NaHCO₃ (sat·aq·, 50 mL) and extracted with EtOAc (50 mL×3). The combined organic layers were washed with brine (50 mL), dried over Na₂SO₄ and concentrated in vacuo. The residue was purified by column chromatography (eluting: EtOAc/PE=0-100%) to afford (R)—N-((8-bromo-6-ethylimidazo[1,2-a]pyridin-2-yl)methyl)-2-methylpropane-2-sulfinamide (200 mg, yield 99%) as pale yellow solid. ESI-MS [M+H]+: 358.1

### Synthesis of (R)—N-((6-ethyl-8-(3-methyl-2,4-dioxoimidazolidin-1-yl)imidazo[1,2-a]pyridin-2-yl)methyl)-2-methylpropane-2-sulfinamide

To a mixture of (R)—N-((8-bromo-6-ethylimidazo[1,2-a]pyridin-2-yl)methyl)-2-methylpropane-2-sulfinamide (380 mg, 1.1 mmol) and 3-methylimidazolidine-2,4-dione (480 mg, 4.2 mmol) in 1,4-dioxane (20 mL) was added Pd₂(dba)₃ (150 mg, 0.16 mmol), Xantphos (180 mg, 0.32 mmol) and Cs₂CO₃ (1.1 mg, 3.2 mmol). The mixture was stirred at 95° C. for 16 h under N₂. The reaction mixture was filtered through Celite® and the filter cake was washed with DCM/MeOH (10/1, 100 mL). The filtrate was concentrated in vacuo to give the crude, which was purified by column chromatography (eluting: MeOH/DCM=0-4%) to afford (R)—N-((6-ethyl-8-(3-methyl-2,4-dioxoimidazolidin-1-yl)imidazo[1,2-a]pyridin-2-yl)methyl)-2-methylpropane-2-sulfinamide (270 mg, yield 64%) as pale yellow solid. ESI-MS [M+H]+: 392.1

### Synthesis of 1-(2-(aminomethyl)-6-ethylimidazo[1,2-a]pyridin-8-yl)-3-methylimidazolidine-2,4-dione

To a mixture of (R)—N-((6-ethyl-8-(3-methyl-2,4-dioxoimidazolidin-1-yl)imidazo[1,2-a]pyridin-2-yl)methyl)-2-methylpropane-2-sulfinamide (270 mg, 0.69 mmol) in DCM (5 mL) was added HCl (4M in dioxane, 1.7 mL, 6.8 mmol). The mixture was stirred at room temperature for 1 h. The mixture was concentrated in vacuo. The residue was treated with NH₃ (7M in MeOH, 5 mL) and the resulting solution was stirred at room temperature for 5 min then concentrated in vacuo. The residue was purified by column chromatography (eluting: MeOH/DCM=0-20%) to afford 1-(2-(aminomethyl)-6-ethylimidazo[1,2-a]pyridin-8-yl)-3-methylimidazolidine-2,4-dione (180 mg, yield 91%) as white solid.

ESI-MS [M+H]+: 288.1

### Synthesis of 1-(2-(1-aminoethyl)-6-chloroimidazo[1,2-a]pyridin-8-yl)-3-methylimidazolidine-2,4-dione

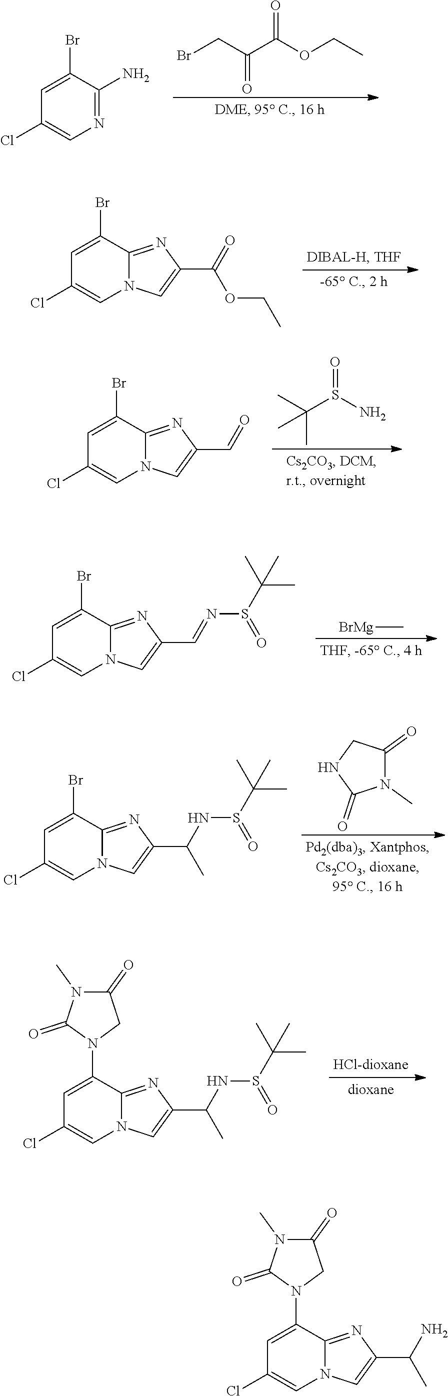

Using a similar procedure to that for (R)-1-(2-(1-aminoethyl)-6-methylimidazo[1,2-a]pyridin-8-yl)-3-methylimidazolidine-2,4-dione, 1-(2-(1-aminoethyl)-6-chloroimidazo[1,2-a]pyridin-8-yl)-3-methylimidazolidine-2,4-dione (0.71 mg) was synthesised from 3-bromo-5-chloropyridin-2-amine. ESI-MS [M+H]⁺: 308.1

### Synthesis of 1-(2-(aminomethyl)-6-chloroimidazo[1,2-a]pyridin-8-yl)-3-methylimidazolidine-2,4-dione

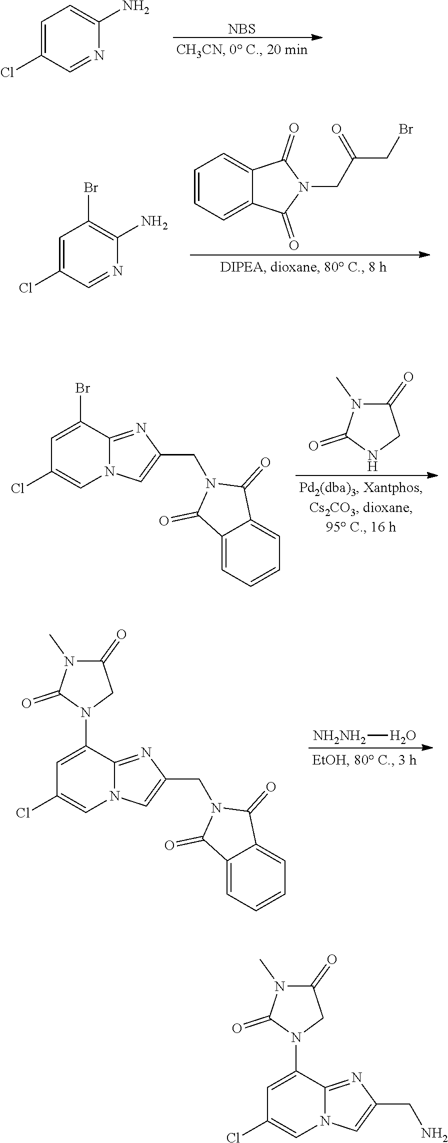

### Synthesis of 3-bromo-5-chloropyridin-2-amine

To a mixture of 5-chloropyridin-2-amine (3.0 g, 23.44 mmol) in CH₃CN (30 mL) was added a solution of NBS (4.2 g, 23.44 mmol) in CH₃CN (20 mL) at 0° C. and stirred for 20 min. The reaction was quenched with water (50 mL) and extracted with EtOAc (3×50 mL). The combined organic layers were washed with brine (50 mL), dried over anhydrous Na₂SO₄ and concentrated in vacuo to give the crude product, which was purified by silica gel chromatography (eluting: EtOAc/PE=1/10) to give 3-bromo-5-chloropyridin-2-amine (3.5 g, 72%) as a brown solid. ESI-MS [M+H]⁺: 207.2.

### Synthesis of 2-((8-bromo-6-chloroimidazo[1,2-a]pyridin-2-yl)methyl)isoindoline-1,3-dione

To a solution of 3-bromo-5-chloropyridin-2-amine (1.0 g, 4.85 mmol) in dioxane (50 mL) was added 2-(3-bromo-2-oxopropyl)isoindoline-1,3-dione (2.7 g, 9.71 mmol) and DIPEA (3.1 g, 24.30 mol). The reaction mixture was stirred at 80° C. for 8h. After cooling to room temperature, the reaction mixture was diluted with EtOAc (100 mL) and washed with brine (50 mL). The organic layer was dried and concentrated in vacuo to give the crude product, which was purified by silica gel chromatography (eluent: PE/EtOAc=10/1) to give 2-((8-bromo-6-chloroimidazo[1,2-a]pyridin-2-yl)methyl)isoindoline-1,3-dione (300 mg, 16%) as a yellow solid. ESI-MS [M+H]⁺: 390.1.

### Synthesis of 2-((6-chloro-8-(3-methyl-2,4-dioxoimidazolidin-1-yl)imidazo[1,2-a]pyridin-2-yl)methyl)isoindoline-1,3-dione

To a mixture of 2-((8-bromo-6-chloroimidazo[1,2-a]pyridin-2-yl)methyl)isoindoline-1,3-dione (200 mg, 0.51 mmol), 3-methylimidazolidine-2,4-dione (116 mg, 1.02 mmol) and Cs₂CO₃ (499 mg, 1.53 mmol) in dioxane (20 mL) was added Pd₂(dba)₃ (46 mg, 0.05 mmol) and Xantphos (58 mg, 0.10 mmol). The reaction mixture was stirred at 95° C. for 16 h, cooled to room temperature, filtered through celite and the filter cake was washed with DCM/MeOH (10/1, 50 mL). The filtrate was concentrated in vacuo to give the crude product, which was purified by silica gel chromatography (eluting: PE/EtOAc=1/2) to give 2-((6-chloro-8-(3-methyl-2,4-dioxoimidazolidin-1-yl)imidazo[1,2-a]pyridin-2-yl)methyl)isoindoline-1,3-dione (150 mg, 69%) as a brown oil. ESI-MS [M+H]+: 424.0.

### Synthesis of 1-(2-(aminomethyl)-6-chloroimidazo[1,2-a]pyridin-8-yl)-3-methylimidazolidine-2,4-dione

To a solution of 2-((6-chloro-8-(3-methyl-2,4-dioxoimidazolidin-1-yl)imidazo[1,2-a]pyridin-2-yl)methyl)isoindoline-1,3-dione (150 mg, 0.35 mmol) in EtOH (15 mL) was added NH₂NH₂—H₂O (0.2 mL). The reaction mixture was stirred at 80° C. for 3 h and then cooled to room temperature. The resulting solution was filtered and filtrate was concentrated in vacuo to give the crude, which was purified by preparative TLC (eluting: DCM/MeOH=10/1) to give 1-(2-(aminomethyl)-6-chloroimidazo[1,2-a]pyridin-8-yl)-3-methylimidazolidine-2,4-dione (60 mg, 58%) as a white solid. ESI-MS [M+H]+: 294.1.

### Synthesis of (6-chloro-8-(4-methylpiperazin-1-yl)imidazo[1,2-a]pyridin-2-yl)methanamine

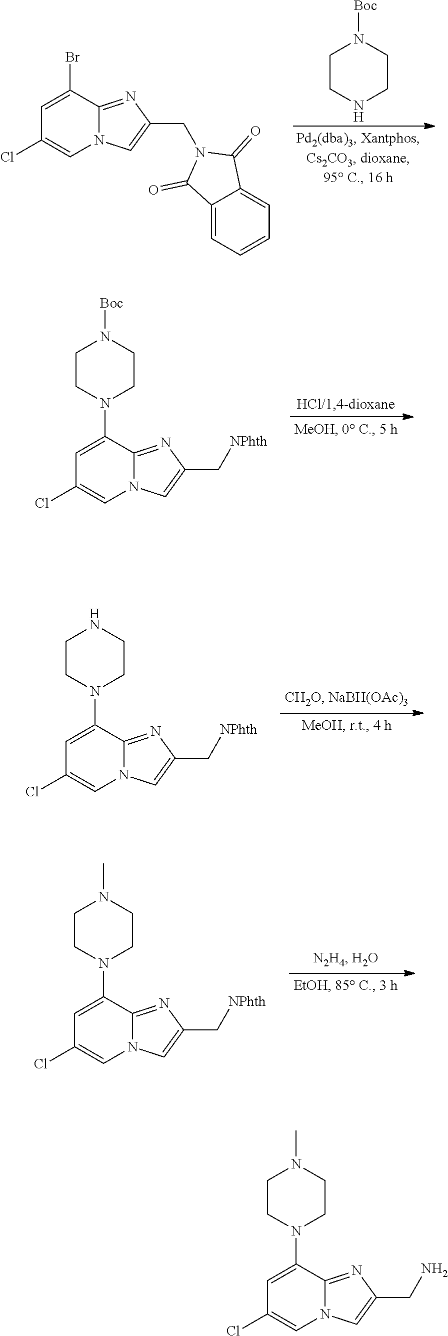

### Synthesis of tert-butyl 4-(6-chloro-2-((1,3-dioxoisoindolin-2-yl)methyl)imidazo[1,2-a]pyridin-8-yl)piperazine-1-carboxylate

To a solution of 2-((8-bromo-6-chloroimidazo[1,2-a]pyridin-2-yl)methyl)isoindoline-1,3-dione (200 mg, 0.51 mmol), tert-butyl piperazine-1-carboxylate (190 mg, 1.02 mmol) and Cs₂CO₃ (499 mg, 1.53 mmol) in dioxane (20 mL) were added Pd₂(dba)₃ (46 mg, 0.05 mmol) and Xantphos (58 mg, 0.10 mmol). The reaction mixture was stirred at 95° C. for 16 h, cooled to room temperature, filtered through celite and the filter cake was washed with DCM/MeOH (10/1, 50 mL). The filtrate was concentrated in vacuo to give the crude product, which was purified by column chromatography (eluting: PE/EtOAc=1/2) to give tert-butyl 4-(6-chloro-2-((1,3-dioxoisoindolin-2-yl)methyl)imidazo[1,2-a]pyridin-8-yl)piperazine-1-carboxylate (151 mg, 60%) as a brown oil. ESI-MS [M+H]+: 496.0

### Synthesis of 2-((6-chloro-8-(piperazin-1-yl)imidazo[1,2-a]pyridin-2-yl)methyl)isoindoline-1,3-dione

To a solution of tert-butyl 4-(6-chloro-2-((1,3-dioxoisoindolin-2-yl)methyl)imidazo[1,2-a]pyridin-8-yl)piperazine-1-carboxylate (0.40 g, 0.81 mmol) in MeOH (10 mL) was added HCl (1.0 mL, 4.0 mmol, 4M in 1,4-dioxane). The mixture was stirred at 0° C. for 5 h and evaporated to give 2-((6-chloro-8-(piperazin-1-yl)imidazo[1,2-a]pyridin-2-yl)methyl)isoindoline-1,3-dione (0.30 g, quant) as a light yellow oil, which was used in next step without purification. ESI-MS [M+H]+: 396.2

### Synthesis of 2-((6-chloro-8-(4-methylpiperazin-1-yl)imidazo[1,2-a]pyridin-2-yl)methyl)isoindoline-1,3-dione

A solution of 2-((6-chloro-8-(piperazin-1-yl)imidazo[1,2-a]pyridin-2-yl)methyl)isoindoline-1,3-dione (0.3 g, crude), formaldehyde (0.19 g, 6.2 mmol, 37 wt. % in H₂O) and NaBH(OAc)₃ (0.92 g, 4.3 mmol) in MeOH (10 mL) was stirred at room temperature for 4 h. Water (20 mL) was added and extracted with EtOAc (3×20 mL). The combined organic layers were washed with brine (50 mL), dried over Na₂SO₄, concentrated in vacuo to give the crude, which was purified by column chromatography on silica gel (eluent: DCM/MeOH=20/1) to give 2-((6-chloro-8-(4-methylpiperazin-1-yl)imidazo[1,2-a]pyridin-2-yl)methyl)isoindoline-1,3-dione (0.15 g, 45%) as a yellow oil. ESI-MS [M+H]+: 410.2

### Synthesis of (6-chloro-8-(4-methylpiperazin-1-yl)imidazo[1,2-a]pyridin-2-yl)methanamine

To a solution of 2-((6-chloro-8-(4-methylpiperazin-1-yl)imidazo[1,2-a]pyridin-2-yl)methyl)isoindoline-1,3-dione (0.15 g, 0.37 mmol)) in EtOH (10 mL) was added N₂H₄·H₂O (92 mg, 1.9 mmol, 80% purity). The mixture was stirred at 85° C. for 3 h and cooled to room temperature. The mixture was filtered, and the filtrate was concentrated in vacuo to give (6-chloro-8-(4-methylpiperazin-1-yl)imidazo[1,2-a]pyridin-2-yl)methanamine (0.12 g, crude) as a yellow oil, which was used in next step without purification.

ESI-MS [M+H]+: 280.2.

### Synthesis of (R)-1-(2-(1-aminoethyl)-6-methoxyimidazo[1,2-a]pyridin-8-yl)-3-methylimidazolidine-2,4-dione

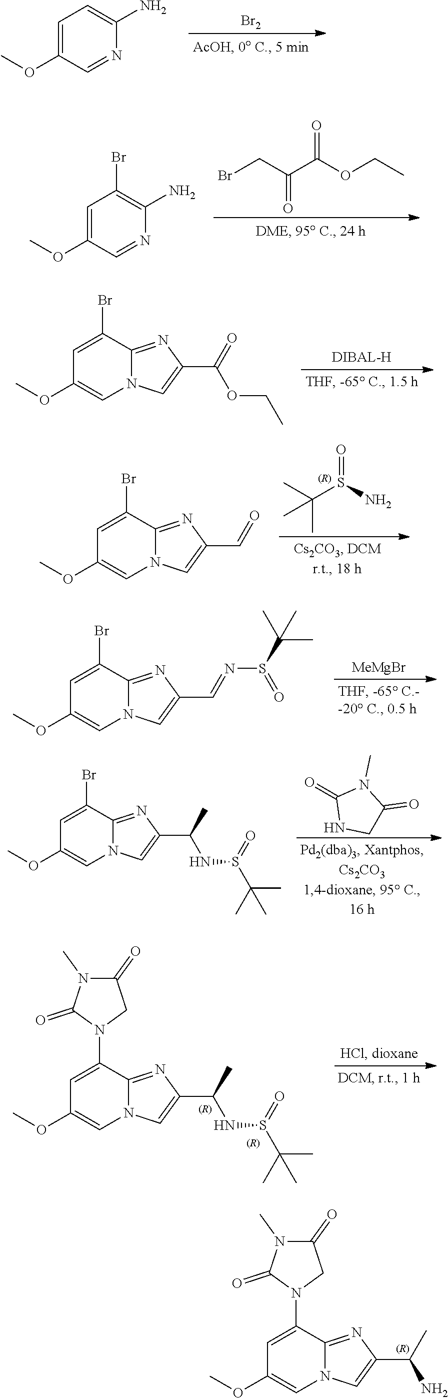

### Synthesis of 3-bromo-5-methoxypyridin-2-amine

To a solution of 5-methoxypyridin-2-amine (5 g, 40 mmol) in AcOH (35 mL) at 0° C. was added Br₂ (2.2 mL, 40 mmol). The mixture was stirred at 0° C. for 5 min and quenched with water (20 mL). The mixture was neutralized with saturated aq·NaHCO₃ (100 mL) and extracted with DCM (3×40 mL). The combined organic layers were washed with brine (200 mL), dried over Na₂SO₄ and concentrated in vacuo. The residue was purified by silica gel column chromatography (eluent: EtOAc/PE=0˜20%) to afford 3-bromo-5-methoxypyridin-2-amine (2.2 g, yield 27%) as brown solid. ESI-MS [M+H]+: 203.1

### Synthesis of ethyl 8-bromo-6-methoxyimidazo[1,2-a]pyridine-2-carboxylate

To a mixture of 3-bromo-5-methoxypyridin-2-amine (2.4 g, 12 mmol) in DME (50 mL) was added ethyl 3-bromo-2-oxopropanoate (3.5 g, 18 mmol). The mixture was stirred at 95° C. for 24 h under N₂ and cooled to room temperature. The mixture was quenched with saturated aq·NaHCO₃ (50 mL) and extracted with DCM (3×50 mL). The combined organic layers were washed with brine (50 mL), dried over Na₂SO₄ and concentrated in vacuo. The residue was purified by flash column chromatography (eluent: EtOAc/PE=0˜40%) to afford ethyl 8-bromo-6-methoxyimidazo[1,2-a]pyridine-2-carboxylate (2.1 g, yield 59%) as brown solid. ESI-MS [M+H]+: 299.1

### Synthesis of 8-bromo-6-methoxyimidazo[1,2-a]pyridine-2-carbaldehyde

To a mixture of ethyl 8-bromo-6-methoxyimidazo[1,2-a]pyridine-2-carboxylate (2.1 g, 7.0 mmol) in THF (40 mL) was added dropwise DIBAL-H (1M in hexane, 22 mL, 22 mmol) at −65° C. After stirring at −65° C. for 1.5 h, the mixture was quenched with NaOH (15%, aq·40 mL) and extracted with EtOAc (3×40 mL). The combined organic layers were washed with brine (50 mL), dried over Na₂SO₄ and concentrated in vacuo. The residue was purified by silica gel column chromatography (eluent: EtOAc/PE=0˜50%) to afford 8-bromo-6-methoxyimidazo[1,2-a]pyridine-2-carbaldehyde (1.1 g, yield 62%) as white solid. ESI-MS [M+H]+: 255.1

### Synthesis of (R,E)-N-((8-bromo-6-methoxyimidazo[1,2-a]pyridin-2-yl)methylene)-2-methylpropane-2-sulfinamide

To a mixture of 8-bromo-6-methoxyimidazo[1,2-a]pyridine-2-carbaldehyde (1.1 g, 4.3 mmol) and (R)-2-methylpropane-2-sulfinamide (0.63 g, 5.2 mmol) in DCM (25 mL) was added Cs₂CO₃ (2.8 g, 8.6 mmol). After stirring at room temperature for 18 h, the mixture was quenched with water (50 mL) and extracted with EtOAc (3×40 mL). The combined organic layers were washed with brine (50 mL), dried over Na₂SO₄ and concentrated in vacuo. The residue was purified by silica gel column chromatography (eluent: EtOAc/PE=0˜50%) to afford (R,E)-N-((8-bromo-6-methoxyimidazo[1,2-a]pyridin-2-yl)methylene)-2-methylpropane-2-sulfinamide (0.56 g, yield 36%) as pale-yellow solid. ESI-MS [M+H]+: 358.1

### Synthesis of (R)—N—((R)-1-(8-bromo-6-methoxyimidazo[1,2-a]pyridin-2-yl)ethyl)-2-methylpropane-2-sulfinamide

To a mixture of (R,E)-N-((8-bromo-6-methoxyimidazo[1,2-a]pyridin-2-yl)methylene)-2-methylpropane-2-sulfinamide (0.56 g, 1.7 mmol) in THF (20 mL) was added methylmagnesium bromide (1.7 mL, 5.1 mmol, 3M in ether) at −65° C. After warming to −20° C. in 30 min, the mixture was quenched with saturated aq·NH₄Cl (30 mL) and extracted with EtOAc (3×30 mL). The combined organic layers were washed with brine (50 mL), dried over Na₂SO₄ and concentrated in vacuo. The residue was purified by silica gel column chromatography (eluent: MeOH/DCM=0˜5%) to afford (R)—N—((R)-1-(8-bromo-6-methoxyimidazo[1,2-a]pyridin-2-yl)ethyl)-2-methylpropane-2-sulfinamide (0.54 g, yield 85%) as pale-yellow solid. ESI-MS [M+H]+: 374.1

### Synthesis of (R)—N—((R)-1-(6-methoxy-8-(3-methyl-2,4-dioxoimidazolidin-1-yl)imidazo[1,2-a]pyridin-2-yl)ethyl)-2-methylpropane-2-sulfinamide

A mixture of (R)—N—((R)-1-(8-bromo-6-methoxyimidazo[1,2-a]pyridin-2-yl)ethyl)-2-methylpropane-2-sulfinamide (0.54 g, 1.5 mmol), 3-methylimidazolidine-2,4-dione (0.83 g, 7.3 mmol), Pd₂(dba)₃ (0.20 g, 0.22 mmol), Xantphos (0.25 g, 0.44 mmol) and Cs₂CO₃ (1.2 mg, 3.6 mmol) in 1,4-dioxane (20 mL) was stirred at 95° C. for 16 h under N₂. The mixture was filtered through Celite® and the filter cake was washed with DCM/MeOH (10/1, 50 mL). The filtrate was concentrated in vacuo to give the crude, which was purified by silica gel chromatography (eluent: MeOH/DCM=0˜4%) to afford (R)—N—((R)-1-(6-methoxy-8-(3-methyl-2,4-dioxoimidazolidin-1-yl)imidazo[1,2-a]pyridin-2-yl)ethyl)-2-methylpropane-2-sulfinamide (0.55 g, yield 92%) as pale-yellow solid. ESI-MS [M+H]+: 408.2

### Synthesis of (R)-1-(2-(1-aminoethyl)-6-methoxyimidazo[1,2-a]pyridin-8-yl)-3-methylimidazolidine-2,4-dione

To a mixture of (R)—N—((R)-1-(6-methoxy-8-(3-methyl-2,4-dioxoimidazolidin-1-yl)imidazo[1,2-a]pyridin-2-yl)ethyl)-2-methylpropane-2-sulfinamide (0.55 g, 1.4 mmol) in DCM (5.0 mL) was added HCl (3.3 mL, 14 mmol, 4M in 1,4-dioxane,). The mixture was stirred at room temperature for 1 h and concentrated in vacuo. NH₃/MeOH (10 mL, 7M in MeOH) was added to the resulting residue and the mixture stirred for 15 min. The mixture was concentrated in vacuo and the residue was purified by silica gel chromatography (eluent: MeOH/DCM=0˜18%) to afford (R)-1-(2-(1-aminoethyl)-6-methoxyimidazo[1,2-a]pyridin-8-yl)-3-methylimidazolidine-2,4-dione (0.28 g, yield 69%) as white solid. ESI-MS [M+H]+: 304.1

### Synthesis of (S*)-1-(2-(1-aminoethyl)-6-(difluoromethyl)imidazo[1,2-a]pyridin-8-yl)-3-methylimidazolidine-2,4-dione

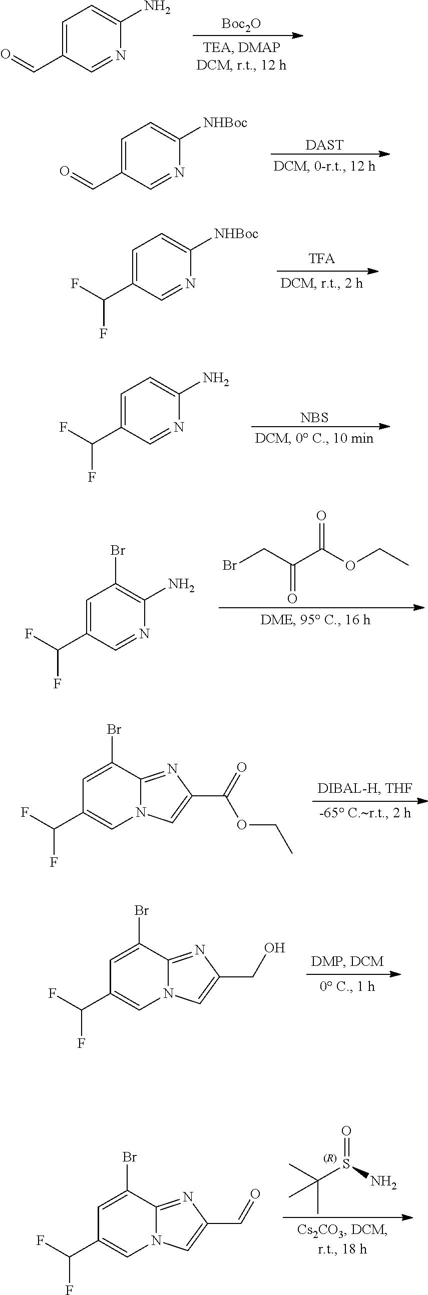

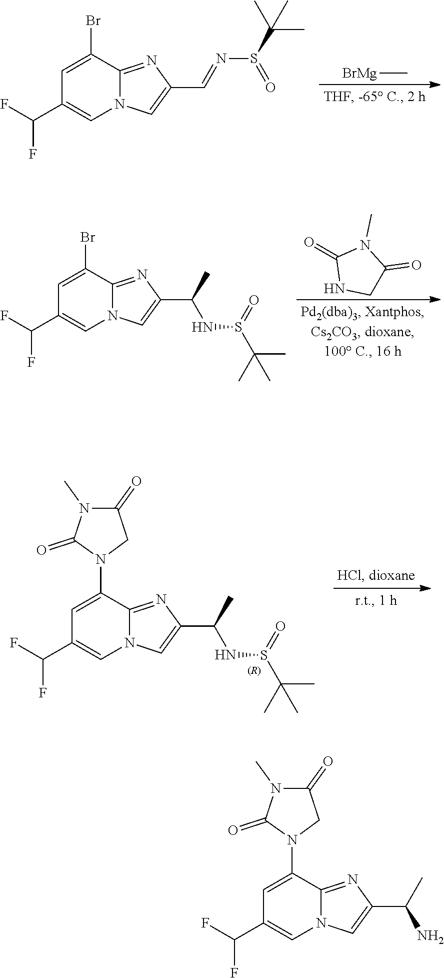

### Synthesis of tert-butyl (5-formylpyridin-2-yl)carbamate

To a mixture of 6-aminonicotinaldehyde (5.0 g, 41 mmol) in DCM (80 mL) were added Boc₂O (9.4 g, 43 mmol), DMAP (0.25 g, 2.0 mmol) at room temperature and the mixture was stirred for 12 h. The mixture was quenched with water (100 mL) and extracted with DCM (3×100 mL). The combined organic layers were washed with brine (100 mL), dried over Na₂SO₄ and concentrated in vacuo. The residue was purified by silica gel column chromatography (eluent: EtOAc/PE from 0 to 20%) to afford tert-butyl (5-formylpyridin-2-yl) carbamate (7.2 g, 79%) as a white solid. ESI-MS [M+H]+: 223.1

### Synthesis of tert-butyl (5-(difluoromethyl) pyridin-2-yl) carbamate

To a mixture of tert-butyl (5-formylpyridin-2-yl) carbamate (7.2 g, 32 mmol) in DCM (100 mL) was added DAST (17 mL, 96 mmol) at 0° C. The mixture was stirred at room temperature for 12 h. The reaction was quenched with saturated aq·NaHCO₃ (100 mL) then extracted with DCM (3×100 mL). The combined organic layers were washed with brine (100 mL), dried over Na₂SO₄ and concentrated in vacuo. The residue was purified by silica gel column chromatography (eluent: EtOAc/PE from 0 to 20%) to afford tert-butyl (5-(difluoromethyl) pyridin-2-yl) carbamate (5.8 g, 76%) as a white solid. ESI-MS [M+H]+: 245.1

### Synthesis of 5-(difluoromethyl)pyridin-2-amine

To a mixture of tert-butyl (5-(difluoromethyl) pyridin-2-yl) carbamate (5.8 g, 24 mmol) in DCM (100 mL) was added TFA (17 mL, 240 mmol) at room temperature. The mixture was stirred at room temperature for 2 h. The reaction was quenched with NH₃/MeOH (7 M in MeOH, 20 mL) until pH>7 and then water (100 mL) was added. The mixture was extracted with DCM (3×100 mL). The combined organic layers were washed with brine (100 mL), dried over Na₂SO₄ and concentrated in vacuo. The residue was purified by silica gel column chromatography (eluent: EtOAc/PE from 0 to 30%) to give 5-(difluoromethyl)pyridin-2-amine (3.5 g, 95%) as a white solid. ESI-MS [M+H]+: 145.0

### Synthesis of 3-bromo-5-(difluoromethyl)pyridin-2-amine

NBS (4.8 g, 27 mmol) was added to a solution of 5-(difluoromethyl) pyridin-2-amine (3.5 g, 24 mmol) in DCM (30 mL) at 0° C. After stirring at 0° C. for 10 min, the mixture was quenched with water (100 mL), and then neutralized with saturated aq·NH₄Cl (100 mL). The aqueous phase was extracted with DCM (3×100 mL). The combined organic layers were washed with brine (100 mL), dried over Na₂SO₄ and concentrated in vacuo. The residue was purified by silica gel column chromatography (eluent: EtOAc/PE from 0 to 20%) to afford 3-bromo-5-(difluoromethyl)pyridin-2-amine (3.2 g, yield 58%) as brown solid. ESI-MS [M+H]+: 223.1

### Synthesis of ethyl 8-bromo-6-(difluoromethyl)imidazo[1,2-a]pyridine-2-carboxylate

To a solution of 3-bromo-5-(difluoromethyl)pyridin-2-amine (3.2 g, 14.4 mmol) in DME (50 mL) was added ethyl 3-bromo-2-oxopropanoate (4.5 g, 24 mmol). After stirring stirred at 95° C. for 24 h under N₂, the reaction mixture was cooled to room temperature. The mixture was then quenched with water (100 mL) and extracted with EtOAc (3×100 mL). The combined organic layers were washed with brine (100 mL), dried over Na₂SO₄ and concentrated in vacuo. The residue was purified by silica gel column chromatography (eluent: EtOAc/PE from 0 to 40%) to afford ethyl 8-bromo-6-(difluoromethyl)imidazo[1,2-a]pyridine-2-carboxylate (2.0 g, yield 41%) as brown solid. ESI-MS [M+H]+: 319.1

### Synthesis of (8-bromo-6-(difluoromethyl)imidazo[1,2-a]pyridin-2-yl)methanol

DIBAL-H (19 mL, 19 mmol) was added dropwise to a solution of ethyl 8-bromo-6-(difluoromethyl)imidazo[1,2-a]pyridine-2-carboxylate (2.0 g, 6.3 mmol) in THF (40 mL) at −65° C. The reaction mixture was further stirred at −65° C. for 1 h and then at room temperature for another 1 h. The mixture was quenched with NaOH (aq·, 15%, 40 mL) and extracted with EtOAc (3×40 mL). The combined organic layers were washed with brine (100 mL), dried over Na₂SO₄ and concentrated in vacuo. The residue was purified by silica gel column chromatography (eluent: EtOAc/PE from 0 to 50%) to afford (8-bromo-6-(difluoromethyl)imidazo[1,2-a]pyridin-2-yl)methanol (1.6 g, yield 92%) as white solid. ESI-MS [M+H]+: 277.1

### Synthesis of 8-bromo-6-(difluoromethyl)imidazo[1,2-a]pyridine-2-carbaldehyde

Dess Martin periodinane (6.1 g, 15 mmol) was added to a solution of (8-bromo-6-(difluoromethyl)imidazo[1,2-a]pyridin-2-yl)methanol (1.6 g, 5.8 mmol) at 0° C. After stirring at 0° C. for 1 h, the reaction was quenched with saturated aq·Na₂S₂O₃ (40 mL). The mixture was cooled to 0° C. and basified with saturated aq·NaHCO₃ (40 mL) until pH>7. The resulting mixture was the extracted with DCM (3×50 mL). The combined organic layers were washed with brine (50 mL), dried over Na₂SO₄ and concentrated in vacuo. The residue was purified by silica gel column chromatography (eluent: EtOAc/PE from 0 to 50%) to afford 8-bromo-6-(difluoromethyl)imidazo[1,2-a]pyridine-2-carbaldehyde (1.4 g, yield 92%) as white solid. ESI-MS [M+H]+: 275.1

### Synthesis of (R,E)-N-((8-bromo-6-(difluoromethyl)imidazo[1,2-a]pyridin-2-yl)methylene)-2-methylpropane-2-sulfinamide

To a mixture of 8-bromo-6-(difluoromethyl)imidazo[1,2-a]pyridine-2-carbaldehyde (1.4 g, 5.0 mmol) and (R)-2-methylpropane-2-sulfinamide (0.72 g, 6.0 mmol) in DCM (30 mL) was added Cs₂CO₃ (3.2 g, 10 mmol). After stirring at room temperature for 18 h, the mixture was filtered through Celite® and the filter cake was washed with DCM/MeOH (10/1, 50 mL). The filtrate was concentrated in vacuo to give the crude, which was purified by silica gel column chromatography (eluent: EtOAc/PE from 0 to 50%) to afford (R,E)-N-((8-bromo-6-(difluoromethyl)imidazo[1,2-a]pyridin-2-yl)methylene)-2-methylpropane-2-sulfinamide (1.4 g, yield 74%) as pale-yellow solid. ESI-MS [M+H]+: 378.1

### Synthesis of (R)—N—((R)-1-(8-bromo-6-(difluoromethyl)imidazo[1,2-a]pyridin-2-yl)ethyl)-2-methylpropane-2-sulfinamide and (S)—N—((R)-1-(8-bromo-6-(difluoromethyl)imidazo[1,2-a]pyridin-2-yl)ethyl)-2-methylpropane-2-sulfinamide

Methylmagnesium bromide ((2.5 mL, 7.4 mmol, 3 M in ether) was added dropwise to a mixture of (R,E)-N-((8-bromo-6-(difluoromethyl)imidazo[1,2-a]pyridin-2-yl)methylene)-2-methylpropane-2-sulfinamide (1.4 g, 3.7 mmol) in THF (40 mL) at −65° C. After warming to −20° C. for 30 min, the mixture was quenched with saturated aq·NH₄Cl (50 mL) and extracted with EtOAc (3×50 mL). The combined organic layers were washed with brine (100 mL), dried over Na₂SO₄ and concentrated in vacuo. The residue was purified by silica gel chromatography (eluent: MeOH/DCM from 0 to 8%) to afford isomer 1 (0.5 g, yield 34%) and isomer 2 (0.5 g, yield 34%) as pale yellow solids (stereochemistry not assigned). ESI-MS [M+H]+: 394.1

The following 2 steps were carried out on isomer 1 and isomer 2 of 1-(8-bromo-6-(difluoromethyl)imidazo[1,2-a]pyridin-2-yl)ethyl)-2-methylpropane-2-sulfinamide-isomer 1 is shown in full, the yield for isomer 2 (after step 2) is given also.

### Synthesis of (S*)—N—((R)-1-(6-(difluoromethyl)-8-(3-methyl-2,4-dioxoimidazolidin-1-yl)imidazo[1,2-a]pyridin-2-yl)ethyl)-2-methylpropane-2-sulfinamide, isomer 1

A mixture of (S*)—N—((R)-1-(8-bromo-6-(difluoromethyl)imidazo[1,2-a]pyridin-2-yl)ethyl)-2-methylpropane-2-sulfinamide (0.20 g, 0.5 mmol, isomer 1), 3-methylimidazolidine-2,4-dione (0.23 g, 2.0 mmol), Pd₂(dba)₃ (0.069 g, 0.075 mmol), Xantphos (0.087 g, 0.15 mmol) and Cs₂CO₃ (0.49 g, 1.5 mmol) in 1,4-dioxane (10 mL) was stirred at 95° C. for 16 h under N₂. The mixture was cooled to room temperature and filtered through Celite®. The filter cake was washed with DCM/MeOH (10/1, 50 mL) and the filtrate was concentrated in vacuo to give the crude, which was purified by silica gel chromatography (eluent: DCM/MeOH=0˜5%) to afford (S*)—N—((R)-1-(6-(difluoromethyl)-8-(3-methyl-2,4-dioxoimidazolidin-1-yl)imidazo[1,2-a]pyridin-2-yl)ethyl)-2-methylpropane-2-sulfinamide, isomer 1 (40 mg, yield 18%) as pale-yellow solid. ESI-MS [M+H]+: 428.1

### Synthesis of (S*)-1-(2-(1-aminoethyl)-6-(difluoromethyl)imidazo[1,2-a]pyridin-8-yl)-3-methylimidazolidine-2,4-dione

To a mixture of (S*)—N—((R)-1-(6-(difluoromethyl)-8-(3-methyl-2,4-dioxoimidazolidin-1-yl)imidazo[1,2-a]pyridin-2-yl)ethyl)-2-methylpropane-2-sulfinamide (40 mg, 0.09 mmol) in DCM (5.0 mL) was added HCl (3.3 mL, 14 mmol, 4M in 1,4-dioxane). The mixture was stirred at room temperature for 1 h and concentrated in vacuo. NH₃/MeOH (7M in MeOH, 10 mL) was added to the resulting residue and the mixture was stirred for 15 min. The solution was concentrated in vacuo and the residue was purified by preparative TLC to afford (S*)-1-(2-(1-aminoethyl)-6-(difluoromethyl)imidazo[1,2-a]pyridin-8-yl)-3-methylimidazolidine-2,4-dione (30 mg, yield 91%) as white solid. ESI-MS [M+H]+: 324.1.1

Isomer 2 of 1-(8-bromo-6-(difluoromethyl)imidazo[1,2-a]pyridin-2-yl)ethyl)-2-methylpropane-2-sulfinamide was also carried through these steps to give the desired product in 30% yield.

### Synthesis of 4-(2-(aminomethyl)-6-cyclopropylimidazo[1,2-a]pyridin-8-yl)-6-methyl-4,6-diazaspiro[2.4]heptane-5,7-dione

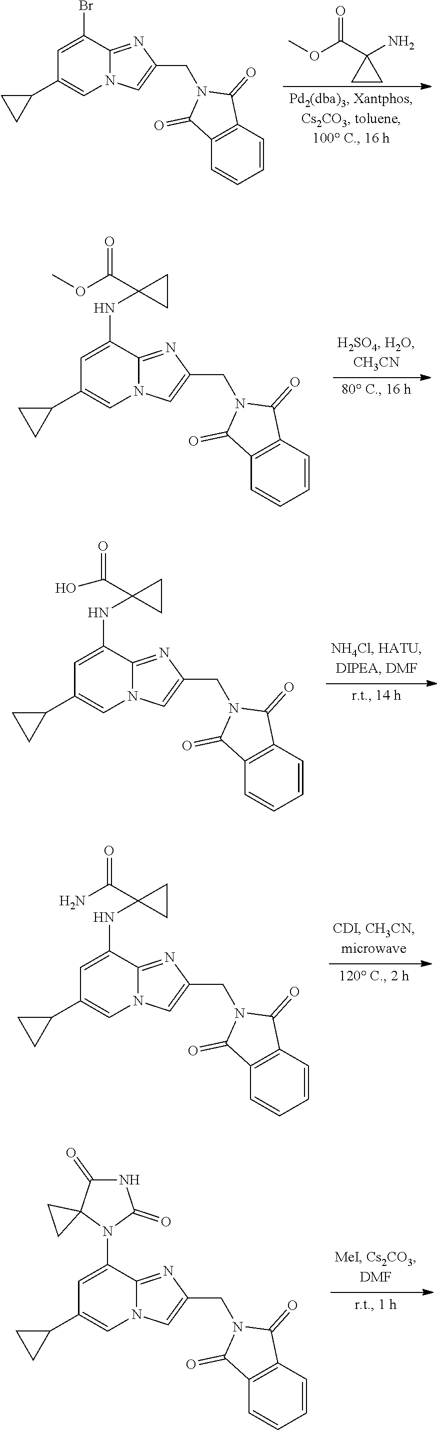

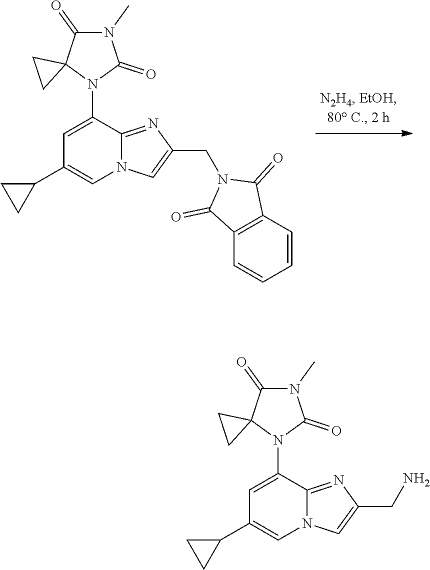 [SMILES: CN1C(=O)N(c2cc(C3CC3)cn3cc(CN4C(=O)c5ccccc5C4=O)nc23)C2(CC2)C1=O ; CN1C(=O)N(c2cc(C3CC3)cn3cc(CN)nc23)C2(CC2)C1=O]

### Synthesis of methyl 1-((6-cyclopropyl-2-((1,3-dioxoisoindolin-2-yl)methyl)imidazo[1,2-a]pyridin-8-yl)amino)cyclopropane-1-carboxylate

To a mixture of 2-((8-bromo-6-cyclopropylimidazo[1,2-a]pyridin-2-yl)methyl)isoindoline-1,3-dione (2 g, 5.05 mmol), methyl 1-aminocyclopropane-1-carboxylate (2.91 g, 25.3 mmol) and Cs₂CO₃ (4.94 g, 15.2 mmol) in toluene (40 mL) was added Pd₂(dba)₃ (460 mg, 0.505 mmol) and Xantphos (580 mg, 1.01 mmol). The mixture was stirred at 100° C. for 16 h under N₂. The reaction mixture was cooled to room temperature, filtered through celite and the filter cake was washed with DCM/MeOH (10/1, 100 mL). The filtrate was concentrated in vacuo to give the crude product, which was purified by silica gel chromatography (eluent: EtOAc/PE=1/2) to give methyl 1-((6-cyclopropyl-2-((1,3-dioxoisoindolin-2-yl)methyl)imidazo[1,2-a]pyridin-8-yl)amino)cyclopropane-1-carboxylate (730 mg, 34) as a yellow solid. ESI-MS [M+H]⁺: 431.1.

### Synthesis of 1-((6-cyclopropyl-2-((1,3-dioxoisoindolin-2-yl)methyl)imidazo[1,2-a]pyridin-8-yl)amino)cyclopropane-1-carboxylic acid

A mixture of methyl 1-((6-cyclopropyl-2-((1,3-dioxoisoindolin-2-yl)methyl)imidazo[1,2-a]pyridin-8-yl)amino)cyclopropane-1-carboxylate (730 mg, 1.70 mmol) and H₂SO₄ (3 mL, 300%) in CH₃CN (6 mL) was stirred at 80° C. for 16 h. The reaction mixture was cooled to room temperature and poured into water (50 mL). The pH was adjusted to 4-5 by NaHCO₃ aqueous (40 mL) and extracted with EtOAc (3×50 ml). The combined organics were washed with brine (100 mL×1), dried over Na₂SO₄, concentrated and dried in vacuo to give 1-((6-cyclopropyl-2-((1,3-dioxoisoindolin-2-yl)methyl)imidazo[1,2-a]pyridin-8-yl)amino)cyclopropane-1-carboxylic acid (390 mg, 550%) as a yellow syrup. ESI-MS [M+H]⁺: 417.2.

### Synthesis of 1-((6-cyclopropyl-2-((1,3-dioxoisoindolin-2-yl)methyl)imidazo[1,2-a]pyridin-8-yl)amino)cyclopropane-1-carboxamide

To a stirred solution of 1-((6-cyclopropyl-2-((1,3-dioxoisoindolin-2-yl)methyl)imidazo[1,2-a]pyridin-8-yl)amino)cyclopropane-1-carboxylic acid (370 mg, 0.888 mmol) and NH₄Cl (475 mg, 8.88 mmol) in DMF (10 mL) was added HATU (439 mg, 1.15 mmol) and DIPEA (344 mg, 2.66 mmol). The resulting mixture was stirred at room temperature for 14 h. The reaction mixture was poured into water (100 mL) and extracted with EtOAc (3×50 ml). The combined organics were washed with brine (3×30 mL), dried over Na₂SO₄ then concentrated in vacuo to give the crude product, which was purified by silica gel chromatography (eluent: DCM/MeOH=30/1) to give 1-((6-cyclopropyl-2-((1,3-dioxoisoindolin-2-yl)methyl)imidazo[1,2-a]pyridin-8-yl)amino)cyclopropane-1-carboxamide (330 mg, 89%) as a pale solid. ESI-MS [M+H]⁺: 416.1.

### Synthesis of 2-((6-cyclopropyl-8-(5,7-dioxo-4,6-diazaspiro[2.4]heptan-4-yl)imidazo[1,2-a]pyridin-2-yl)methyl)isoindoline-1,3-dione

A mixture of 1-((6-cyclopropyl-2-((1,3-dioxoisoindolin-2-yl)methyl)imidazo[1,2-a]pyridin-8-yl)amino)cyclopropane-1-carboxamide (330 mg, 0.794 mmol), CDI (644 mg, 3.97 mmol) and Et₃N (402 mg, 3.97 mmol) in CH₃CN (8 mL) was stirred in a sealed tube. After degassing with N₂ for 1 min, the reaction was irradiated in microwave at 120° C. for 2 h. After the reaction mixture was cooled to room temperature, water (40 mL) was added then the mixture was extracted with EtOAc (40 ml×3). The combined organics were washed with brine (40 mL), dried over Na₂SO₄ then concentrated in vacuo to give the crude product, which was purified by silica gel chromatography (eluent: DCM/MeOH=30/1) to give 2-((6-cyclopropyl-8-(5,7-dioxo-4,6-diazaspiro[2.4]heptan-4-yl)imidazo[1,2-a]pyridin-2-yl)methyl)isoindoline-1,3-dione (350 mg, 100%) as a yellow solid. ESI-MS [M+H]⁺: 442.1.

### Synthesis of 2-((6-cyclopropyl-8-(6-methyl-5,7-dioxo-4,6-diazaspiro[2.4]heptan-4-yl)imidazo[1,2-a]pyridin-2-yl)methyl)isoindoline-1,3-dione

To a stirred solution of 2-((6-cyclopropyl-8-(5,7-dioxo-4,6-diazaspiro[2.4]heptan-4-yl)imidazo[1,2-a]pyridin-2-yl)methyl)isoindoline-1,3-dione (350 mg, 0.793 mmol) and Cs₂CO₃ (517 mg, 1.586 mmol) in DMF (10 mL) was added MeI (225 mg, 1.586 mmol). The mixture was stirred at room temperature for 1 h. The reaction mixture was poured into water (50 mL) and extracted with EtOAc (3×50 ml). The combined organics were washed with brine (50 mL), dried over Na₂SO₄ then concentrated in vacuo to give the crude, which was purified by silica gel chromatography (eluent: EtOAc/PE=1/2) to give 2-((6-cyclopropyl-8-(6-methyl-5,7-dioxo-4,6-diazaspiro[2.4]heptan-4-yl)imidazo[1,2-a]pyridin-2-yl)methyl)isoindoline-1,3-dione (250 mg, 69%) as a pale solid. ESI-MS [M+H]⁺: 456.1.

### Synthesis of 4-(2-(aminomethyl)-6-cyclopropylimidazo[1,2-a]pyridin-8-yl)-6-methyl-4,6-diazaspiro[2.4]heptane-5,7-dione

To a stirred solution of 2-((6-cyclopropyl-8-(6-methyl-5,7-dioxo-4,6-diazaspiro[2.4]heptan-4-yl)imidazo[1,2-a]pyridin-2-yl)methyl)isoindoline-1,3-dione (250 mg, 0.549 mmol) in EtOH (5 mL) was added N₂H₄ (2 mL, 80% in water). The mixture was stirred at 80° C. for 2 h, cooled to room temperature, filtered and the filtrate was concentrated to give the crude product, which was purified by preparative TLC (DCM/MeOH/NH₃=10/1/1) to give 4-(2-(aminomethyl)-6-cyclopropylimidazo[1,2-a]pyridin-8-yl)-6-methyl-4,6-diazaspiro[2.4]heptane-5,7-dione (110 mg, 61%) as a pale solid. ESI-MS [M+H]⁺: 326.1.

### Synthesis of 7-(2-(aminomethyl)-6-cyclopropylimidazo[1,2-a]pyridin-8-yl)-2,5-dioxa-7-azaspiro[3.4]octan-6-one

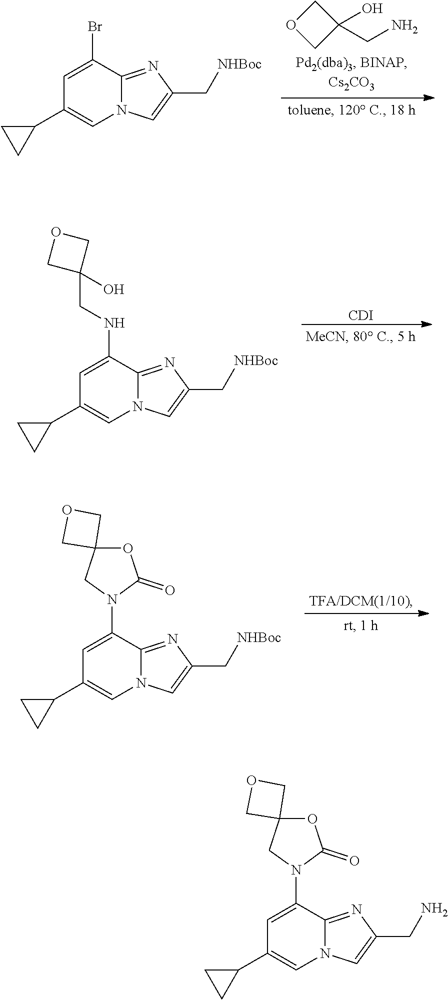 [SMILES: CC(C)(C)OC(=O)NCc1cn2cc(C3CC3)cc(Br)c2n1 ; NCC1(O)COC1 ; CC(C)(C)OC(=O)NCc1cn2cc(C3CC3)cc(NCC3(O)COC3)c2n1 ; "CDI" ; CC(C)(C)OC(=O)NCc1cn2cc(C3CC3)cc(N3CC4(COC4)OC3=O)c2n1 ; NCc1cn2cc(C3CC3)cc(N3CC4(COC4)OC3=O)c2n1]

### Synthesis of tert-butyl ((6-cyclopropyl-8-(((3-hydroxyoxetan-3-yl)methyl)amino)imidazo[1,2-a]pyridin-2-yl)methyl)carbamate

A mixture of tert-butyl ((8-bromo-6-cyclopropylimidazo[1,2-a]pyridin-2-yl)methyl)carbamate (730 mg, 2.0 mmol), 3-(aminomethyl)oxetan-3-ol (206 mg, 2.0 mmol), Pd₂(dba)₃ (275 mg, 0.3 mmol), BINAP (373 mg, 0.6 mmol) and Cs₂CO₃ (1.3 g, 4.0 mmol) in toluene (20 mL) was stirred at 100° C. for 18 h under N₂. The reaction was cooled to room temperature, quenched with water (30 mL) and extracted with EtOAc (3×30 mL). The combined organic layers were washed with brine (20 mL), dried over anhydrous Na₂SO₄ and concentrated in vacuo to give the crude product, which was purified by silica gel chromatography (eluent: DCM/MeOH=50/1˜20/1) to afford tert-butyl ((6-cyclopropyl-8-(((3-hydroxyoxetan-3-yl)methyl)amino)imidazo[1,2-a]pyridin-2-yl)methyl)carbamate (220 mg, 28%) as a yellow oil. ESI-MS [M+H]⁺: 389.2.

### Synthesis of tert-butyl ((6-cyclopropyl-8-(6-oxo-2,5-dioxa-7-azaspiro[3.4]octan-7-yl)imidazo[1,2-a]pyridin-2-yl)methyl)carbamate

A mixture of tert-butyl ((6-cyclopropyl-8-(((3-hydroxyoxetan-3-yl)methyl)amino)imidazo[1,2-a]pyridin-2-yl)methyl)carbamate (220 mg, 0.57 mmol) and CDI (367 mg, 2.27 mmol) in MeCN (20 mL) was stirred at 80° C. for 5h. The reaction was cooled to room temperature, quenched with water (30 mL) and extracted with EtOAc (3×30 mL). The combined organic layers were washed with brine (30 mL), dried over Na₂SO₄ and concentrated in vacuo to give the crude product, which purified by preparative TLC (eluent: MeOH/DCM=1/15) to afford tert-butyl ((6-cyclopropyl-8-(6-oxo-2,5-dioxa-7-azaspiro[3.4]octan-7-yl)imidazo[1,2-a]pyridin-2-yl)methyl)carbamate (180 mg, 76%) as a white solid. ESI-MS [M+H]⁺: 415.2.

### Synthesis of 7-(2-(aminomethyl)-6-cyclopropylimidazo[1,2-a]pyridin-8-yl)-2,5-dioxa-7-azaspiro[3.4]octan-6-one

A solution of tert-butyl ((6-cyclopropyl-8-(6-oxo-2,5-dioxa-7-azaspiro[3.4]octan-7-yl)imidazo[1,2-a]pyridin-2-yl)methyl)carbamate (120 mg, 0.29 mmol) in TFA/DCM (0.5 mL/5.0 mL) was stirred at room temperature for 1 h. The mixture was quenched with sat·NaHCO₃ solution (30 mL) then extracted with DCM (3×10 mL). The combined organic layers were washed with brine (30 mL), dried over Na₂SO₄ and concentrated in vacuo to give the crude, which was purified by preparative TLC (eluent: MeOH/DCM=1/8) to afford 7-(2-(aminomethyl)-6-cyclopropylimidazo[1,2-a]pyridin-8-yl)-2,5-dioxa-7-azaspiro[3.4]octan-6-one (60 mg, 66%) as a yellow oil. ESI-MS [M+H]⁺: 315.2.

### Synthesis of (R)-7-(2-(1-aminoethyl)-6-cyclopropylimidazo[1,2-a]pyridin-8-yl)-2,5-dimethyl-2,5,7-triazaspiro[3.4]octan-6-one

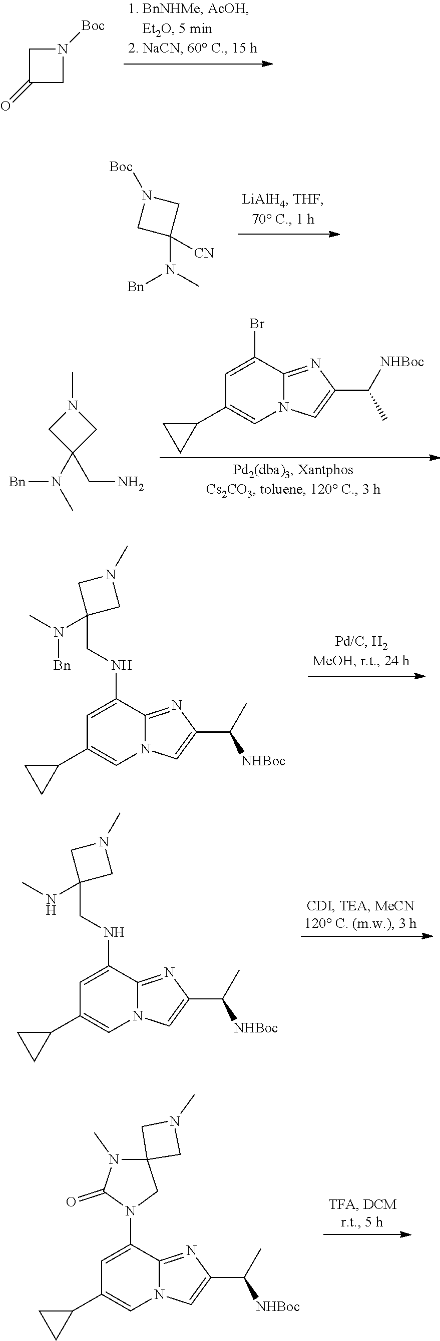

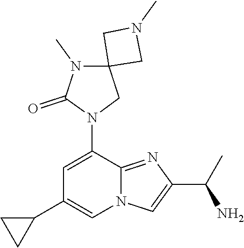 [SMILES: C[C@@H](N)c1cn2cc(C3CC3)cc(N3CC4(CN(C)C4)N(C)C3=O)c2n1]

### Synthesis of tert-butyl 3-(benzyl(methyl)amino)-3-cyanoazetidine-1-carboxylate

A mixture of tert-butyl 3-oxoazetidine-1-carboxylate (3 g, 17.5 mmol), N-methyl-1-phenylmethanamine (2.1 g, 17.5 mmol) and acetic acid (2.1 g, 35 mmol) in Et₂O (100 mL) was stirred at room temperature for 5 min. Then NaCN (857.5 mg, 17.5 mmol) was added and the mixture was stirred at 60° C. for 15h. The reaction was quenched with saturated aq·NaHCO₃ (100 mL) and extracted with EtOAc (3×50 mL). The organic layers were washed with brine (50 mL), dried over Na₂SO₄, concentrated in vacuo to give the crude, which was purified by silica gel chromatography (eluent: PE/EtOAc=0-10%) to give tert-butyl 3-(benzyl(methyl)amino)-3-cyanoazetidine-1-carboxylate (3.8 g, 73%) as a yellow solid. ESI-MS [M+H]+: 557.2

### Synthesis of 3-(aminomethyl)-N-benzyl-N,1-dimethylazetidin-3-amine

To a solution of tert-butyl 3-(benzyl(methyl)amino)-3-cyanoazetidine-1-carboxylate (600 mg, 2.0 mmol) in THF (40 mL) was added LiAlH₄ (10 mL, 10 mmol, 1M in THF). After stirring at 70° C. for 1 h, the reaction was quenched Na₂SO₄-10H₂O at 0° C. The mixture was filtered and washed with MeOH (100 mL). The filtrate was concentrated in vacuo to give the crude, which was purified with preparative TLC (eluent: DMC/MeOH=0˜12%) to give 3-(aminomethyl)-N-benzyl-N,1-dimethylazetidin-3-amine (300 mg, 69%) as a yellow solid. ESI-MS [M+H]+: 220.2

### Synthesis of tert-butyl (R)-(1-(8-(((3-(benzyl(methyl)amino)-1-methylazetidin-3-yl)methyl)amino)-6-cyclopropylimidazo[1,2-a]pyridin-2-yl)ethyl)carbamate

A mixture of 3-(aminomethyl)-N-benzyl-N,1-dimethylazetidin-3-amine (290 mg, 1.32 mmol), tert-butyl (R)-(1-(8-bromo-6-cyclopropylimidazo[1,2-a]pyridin-2-yl)ethyl)carbamate (501 mg, 1.32 mmol), Pd₂(dba)₃ (120 mg, 0.13 mmol), Xantphos (117 mg, 0.2 mmol) and Cs₂CO₃ (1.1 g, 3.3 mmol) in toluene (25 mL) was stirred at 120° C. for 3 h. The reaction was quenched with brine (30 mL) and extracted with EtOAc (3×40 mL). The combined organic layers were washed with brine (40 mL), dried over Na₂SO₄ then concentrated in vacuo to give the crude, which was purified by silica gel chromatography (eluent: DCM/MeOH=0-5%) to give tert-butyl (R)-(1-(8-(((3-(benzyl(methyl)amino)-1-methylazetidin-3-yl)methyl)amino)-6-cyclopropylimidazo[1,2-a]pyridin-2-yl)ethyl)carbamate (180 mg, 26%) as a yellow solid. ESI-MS [M+H]+: 519.2

### Synthesis of tert-butyl (R)-(1-(6-cyclopropyl-8-(((1-methyl-3-(methylamino)azetidin-3-yl)methyl)amino)imidazo[1,2-a]pyridin-2-yl)ethyl)carbamate

A mixture of tert-butyl (R)-(1-(8-(((3-(benzyl(methyl)amino)-1-methylazetidin-3-yl)methyl)amino)-6-cyclopropylimidazo[1,2-a]pyridin-2-yl)ethyl)carbamate (180 mg, 0.35 mmol) and Pd/C (30 mg) in MeOH (20 mL) was stirred at room temperature for 24h under H₂. The reaction mixture was filtered through Celite® and the filter cake was washed with MeOH (50 mL). The filtrate was concentrated in vacuo to give the crude, which was purified by preparative TLC (eluent: DCM/MeOH=10/1) to give tert-butyl (R)-(1-(6-cyclopropyl-8-(((1-methyl-3-(methylamino)azetidin-3-yl)methyl)amino)imidazo[1,2-a]pyridin-2-yl)ethyl)carbamate (60 mg, 40%) as a yellow solid. ESI-MS [M+H]+: 429.2

### Synthesis of tert-butyl (R)-(1-(6-cyclopropyl-8-(2,5-dimethyl-6-oxo-2,5,7-triazaspiro[3.4]octan-7-yl)imidazo[1,2-a]pyridin-2-yl)ethyl)carbamate

A mixture of tert-butyl (R)-(1-(6-cyclopropyl-8-(((1-methyl-3-(methylamino)azetidin-3-yl)methyl)amino)imidazo[1,2-a]pyridin-2-yl)ethyl)carbamate (60 mg, 0.14 mmol), CDI (180 mg, 1.12 mmol) and TEA (170 mg, 1.68 mmol) in MeCN (4 mL) was stirred at 120° C. under microwave heating for 3 h. The reaction was diluted with water (30 mL) and extracted with EtOAc (3×40 mL). The combined organic layers were washed with brine (40 mL), dried over Na₂SO₄ and concentrated in vacuo to give the crude, which was purified with preparative TLC (eluent: DCM/MeOH=10/1) to give tert-butyl (R)-(1-(6-cyclopropyl-8-(2,5-dimethyl-6-oxo-2,5,7-triazaspiro[3.4]octan-7-yl)imidazo[1,2-a]pyridin-2-yl)ethyl)carbamate (40 mg, 63%) as a yellow solid. ESI-MS [M+H]+: 455.2

### Synthesis of (R)-7-(2-(1-aminoethyl)-6-cyclopropylimidazo[1,2-a]pyridin-8-yl)-2,5-dimethyl-2,5,7-triazaspiro[3.4]octan-6-one

To a solution of tert-butyl (R)-(1-(6-cyclopropyl-8-(2,5-dimethyl-6-oxo-2,5,7-triazaspiro[3.4]octan-7-yl)imidazo[1,2-a]pyridin-2-yl)ethyl)carbamate (40 mg, 0.09 mmol) in DCM (5 mL) was added TFA (0.5 mL). The reaction was stirred at room temperature for 5h and concentrated in vacuo to give the crude. The residue was neutralized with NH₃ (7M solution in MeOH) and concentrated in vacuo to give the crude, which was purified with preparative TLC (eluent: DCM/MeOH/NH4OH=10/1/0.1) to give (R)-7-(2-(1-aminoethyl)-6-cyclopropylimidazo[1,2-a]pyridin-8-yl)-2,5-dimethyl-2,5,7-triazaspiro[3.4]octan-6-one (20 mg, 63%) as yellow oil. ESI-MS [M+H]+: 355.2

### Synthesis of 5-(2-(1-aminoethyl)-6-cyclopropylimidazo[1,2-a]pyridin-8-yl)-7-methyl-2-oxa-5,7-diazaspiro[3.4]octane-6,8-dione

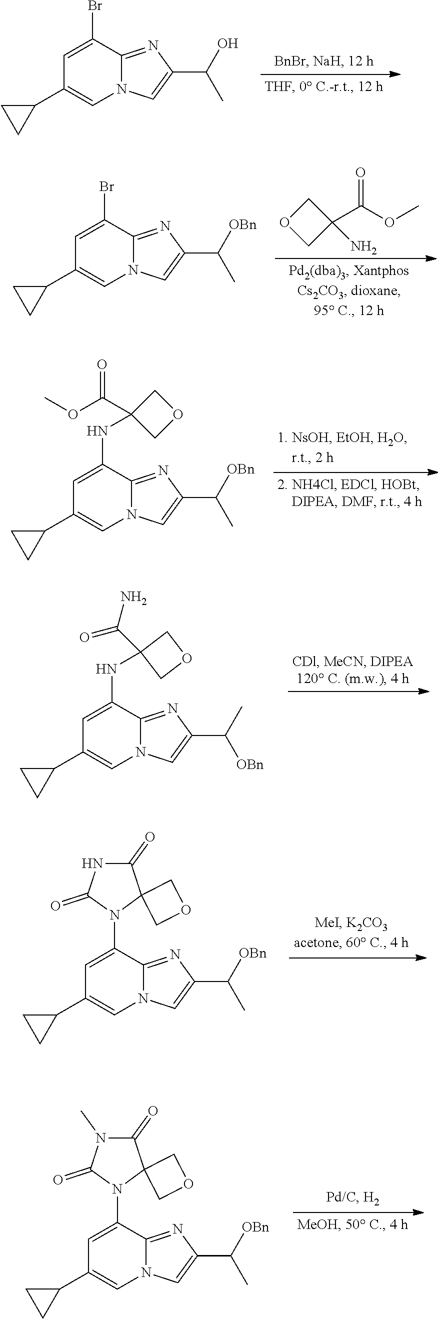

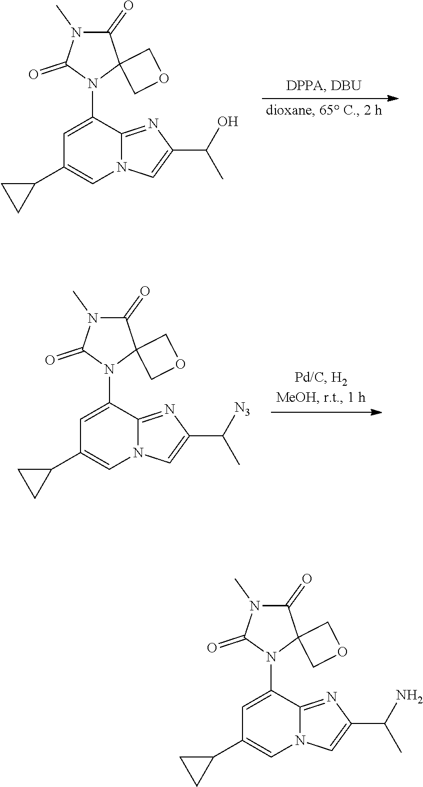 [SMILES: CC(O)c1cn2cc(C3CC3)cc(N3C(=O)N(C)C(=O)C34COC4)c2n1 ; CC(N=[N+]=[N-])c1cn2cc(C3CC3)cc(N3C(=O)N(C)C(=O)C34COC4)c2n1 ; CC(N)c1cn2cc(C3CC3)cc(N3C(=O)N(C)C(=O)C34COC4)c2n1]

### Synthesis of 2-(1-(benzyloxy)ethyl)-8-bromo-6-cyclopropylimidazo[1,2-a]pyridine

To a solution of 1-(8-bromo-6-cyclopropylimidazo[1,2-a]pyridin-2-yl)ethan-1-ol (2.0 g, 7.1 mmol) in THF (60 mL) was slowly added NaH (430 mg, 17.9 mmol) at 0° C. After stirring 30 min, BnCl (1.8 g, 10.5 mmol) was added and the mixture was stirred at room temperature for 12 h. The mixture was quenched with saturated aq·NH₄Cl (100 mL) and extracted with EtOAc (3×50 mL). The combined organic layers were washed with brine (50 mL), dried over Na₂SO₄ and concentrated in vacuo. The residue was purified by silica gel chromatography (eluent:: EtOAc/PE from 0 to 20%) to give 2-(1-(benzyloxy)ethyl)-8-bromo-6-cyclopropylimidazo[1,2-a]pyridine (1.8 g, 68%) as a colorless oil. ESI-MS [M+H]+: 371.1.

### Synthesis of methyl 3-((2-(1-(benzyloxy)ethyl)-6-cyclopropylimidazo[1,2-a]pyridin-8-yl)amino)oxetane-3-carboxylate

A mixture of 2-(1-(benzyloxy)ethyl)-8-bromo-6-cyclopropylimidazo[1,2-a]pyridine (1.8 g, 4.86 mmol), methyl 3-aminooxetane-3-carboxylate (764 mg, 5.83 mmol), Pd₂(dba)₃ (889 mg, 0.97 mmol), xantphos (1.12 g, 1.94 mmol) and Cs₂CO₃ (3.96 g, 12.1 mmol) in 1,4-dioxane (50 mL) was stirred at 95° C. for 12 h under N₂. The reaction mixture was filtered through Celite® and the filter cake was washed with DCM/MeOH (10/1, 50 mL). The filtrate was concentrated in vacuo to give the crude, which was purified by silica gel chromatography (eluent: DCM/MeOH=0-5%) to give methyl 3-((2-(1-(benzyloxy)ethyl)-6-cyclopropylimidazo[1,2-a]pyridin-8-yl)amino)oxetane-3-carboxylate (900 mg, 44%) as a white solid. ESI-MS [M+H]+: 422.2.

### Synthesis of 3-((2-(1-(benzyloxy)ethyl)-6-cyclopropylimidazo[1,2-a]pyridin-8-yl)amino)oxetane-3-carboxamide

To a mixture of methyl 3-((2-(1-(benzyloxy)ethyl)-6-cyclopropylimidazo[1,2-a]pyridin-8-yl)amino)oxetane-3-carboxylate (900 mg, 2.1 mmol) in EtOH/H₂O (12 mL/4 mL) was added LiOH—H₂O (101 mg, 4.2 mmol). After stirring at room temperature for 2 h, the solution was acidified to pH=3-4 with aq·HCl (1N) and extracted with EtOAc (3×50 mL). The combined organic layers were washed with brine (50 mL), dried over Na₂SO₄ and concentrated in vacuo. The crude product was dissolved in DMF (10 mL) and NH₄Cl (597 mg, 11.1 mmol), HOBt (597 mg, 4.4 mmol), EDCI (1.7 g, 8.8 mmol) and DIPEA (1.4 g, 10.9 mmol) were added. After stirring at room temperature for 4h, the mixture was quenched with water (100 mL) and extracted with EtOAc (3×50 mL). The combined organic layers were washed with brine (50 mL), dried over Na₂SO₄ and concentrated in vacuo to give the crude, which was purified by silica gel chromatography (eluent:: DCM/MeOH=0-7%) to give 3-((2-(1-(benzyloxy)ethyl)-6-cyclopropylimidazo[1,2-a]pyridin-8-yl)amino)oxetane-3-carboxamide (780 mg, 90%) as a yellow solid. ESI-MS [M+H]⁺: 407.2.

### Synthesis of 5-(2-(1-(benzyloxy)ethyl)-6-cyclopropylimidazo[1,2-a]pyridin-8-yl)-2-oxa-5,7-diazaspiro[3.4]octane-6,8-dione

To a mixture of 3-((2-(1-(benzyloxy)ethyl)-6-cyclopropylimidazo[1,2-a]pyridin-8-yl)amino)oxetane-3-carboxamide (780 mg, 1.92 mmol) in MeCN (10 mL) was added CDI (1.56 g, 9.6 mmol) and TEA (2.67 mL, 19.2 mmol). After heating at 120° C. for 4h in a microwave, the mixture was washed with water (50 mL,) and extracted with EtOAc (3×30 mL). The combined organic layers were washed with brine (30 mL), dried over Na₂SO₄ and concentrated in vacuo. The residue was purified by silica gel chromatography (eluent: DCM/MeOH=0-5%) to give 5-(2-(1-(benzyloxy)ethyl)-6-cyclopropylimidazo[1,2-a]pyridin-8-yl)-7-methyl-2-oxa-5,7-diazaspiro[3.4]octane-6,8-dione (610 mg, 74%) as a yellow solid. ESI-MS [M+H]+: 433.2.

### Synthesis of 5-(2-(1-(benzyloxy)ethyl)-6-cyclopropylimidazo[1,2-a]pyridin-8-yl)-7-methyl-2-oxa-5,7-diazaspiro[3.4]octane-6,8-dione

To a mixture of 5-(2-(1-(benzyloxy)ethyl)-6-cyclopropylimidazo[1,2-a]pyridin-8-yl)-7-methyl-2-oxa-5,7-diazaspiro[3.4]octane-6,8-dione (610 mg, 1.41 mmol) in acetone (15 mL) were added K₂CO₃ (585 mg, 4.24 mmol) and MeI (602 mg, 4.24 mmol). The mixture was stirred at 60° C. for 4h and cooled to room temperature. The mixture was quenched with water (30 mL,) and extracted with EtOAc (3×30 mL). The combined organic layers were washed with brine (30 mL), dried over Na₂SO₄ and concentrated in vacuo. The residue was purified by silica gel chromatography (eluent: DCM/MeOH=0-5%) to give 5-(2-(1-(benzyloxy)ethyl)-6-cyclopropylimidazo[1,2-a]pyridin-8-yl)-7-methyl-2-oxa-5,7-diazaspiro[3.4]octane-6,8-dione (570 mg, 91%) as a yellow solid. ESI-MS [M+H]+: 447.2.

### Synthesis of 5-(6-cyclopropyl-2-(1-hydroxyethyl)imidazo[1,2-a]pyridin-8-yl)-7-methyl-2-oxa-5,7-diazaspiro[3.4]octane-6,8-dione

A mixture of 5-(2-(1-(benzyloxy)ethyl)-6-cyclopropylimidazo[1,2-a]pyridin-8-yl)-7-methyl-2-oxa-5,7-diazaspiro[3.4]octane-6,8-dione (570 mg, 1.28 mmol) and Pd/C (122 mg) in MeOH (10 mL) was stirred at 50° C. for 4h under H₂. The reaction mixture was filtered through Celite® and the filter cake was washed with DCM/MeOH (10/1, 50 mL). The filtrate was concentrated in vacuo to give the crude, which was purified by silica gel chromatography (eluent: DCM/MeOH=30/1) to give 5-(6-cyclopropyl-2-(1-hydroxyethyl)imidazo[1,2-a]pyridin-8-yl)-7-methyl-2-oxa-5,7-diazaspiro[3.4]octane-6,8-dione (210 mg, 42%) as a white solid. ESI-MS [M+H]+: 357.2.

### Synthesis of 5-(2-(1-azidoethyl)-6-cyclopropylimidazo[1,2-a]pyridin-8-yl)-7-methyl-2-oxa-5,7-diazaspiro[3.4]octane-6,8-dione

To a mixture of 5-(6-cyclopropyl-2-(1-hydroxyethyl)imidazo[1,2-a]pyridin-8-yl)-7-methyl-2-oxa-5,7-diazaspiro[3.4]octane-6,8-dione (160 mg, 0.45 mmol) in 1,4-dioxane (10 mL) were added DPPA (1.24 g, 4.51 mmol) and DBU (684 mg, 4.5 mmol). After stirring at 65° C. for 2 h, the mixture was quenched with water (50 mL) and extracted with EtOAc (3×50 mL). The combined organic layers were washed with brine (50 mL), dried over Na₂SO₄ and concentrated in vacuo. The residue was purified by silica gel column chromatography (eluent: DCM/MeOH=0˜30/1) to give 5-(2-(1-azidoethyl)-6-cyclopropylimidazo[1,2-a]pyridin-8-yl)-7-methyl-2-oxa-5,7-diazaspiro[3.4]octane-6,8-dione (120 mg, 70%) as a grey solid. ESI-MS [M+H]+: 382.2.

### Synthesis of 5-(2-(1-aminoethyl)-6-cyclopropylimidazo[1,2-a]pyridin-8-yl)-7-methyl-2-oxa-5,7-diazaspiro[3.4]octane-6,8-dione

To a mixture of 5-(2-(1-azidoethyl)-6-cyclopropylimidazo[1,2-a]pyridin-8-yl)-7-methyl-2-oxa-5,7-diazaspiro[3.4]octane-6,8-dione (120 mg, 0.31 mmol) in MeOH (10 mL) was added Pd/C (24 mg). After stirring at room temperature for 1 h under H₂, the reaction mixture was filtered through Celite® and the filter cake was washed with DCM/MeOH (10/1, 50 mL). The filtrate was concentrated to give the crude, which was purified by silica gel chromatography (eluent: DCM/NH₃·MeOH=20/1) to give 5-(2-(1-aminoethyl)-6-cyclopropylimidazo[1,2-a]pyridin-8-yl)-7-methyl-2-oxa-5,7-diazaspiro[3.4]octane-6,8-dione (100 mg, 90%) as a white solid. ESI-MS [M+H]+: 356.1.

### Synthesis of 7-(6-cyclopropyl-2-(hydroxymethyl)imidazo[1,2-a]pyridin-8-yl)-2,5-dioxa-7-azaspiro[3.4]octan-6-one

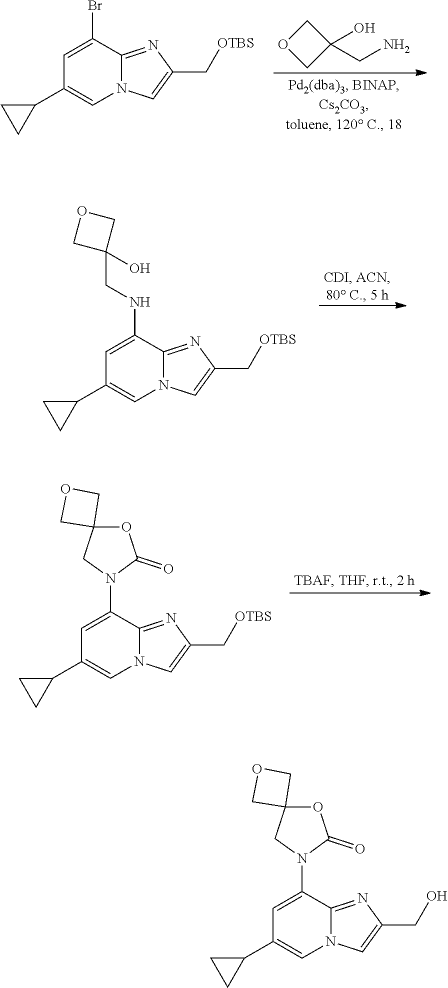 [SMILES: CC(C)(C)[Si](C)(C)OCc1cn2cc(C3CC3)cc(Br)c2n1 ; NCC1(O)COC1 ; CC(C)(C)[Si](C)(C)OCc1cn2cc(C3CC3)cc(NCC3(O)COC3)c2n1 ; "CDI, ACN,80° C., 5 h" ; CC(C)(C)[Si](C)(C)OCc1cn2cc(C3CC3)cc(N3CC4(COC4)OC3=O)c2n1 ; O=C1OC2(COC2)CN1c1cc(C2CC2)cn2cc(CO)nc12]

### Synthesis of 3-(((2-(((tert-butyldimethylsilyl)oxy)methyl)-6-cyclopropylimidazo[1,2-a]pyridin-8-yl)amino)methyl)oxetan-3-ol

To a mixture of 8-bromo-2-(((tert-butyldimethylsilyl)oxy)methyl)-6-cyclopropylimidazo[1,2-a]pyridine (500 mg, 1.31 mmol), 3-(aminomethyl)oxetan-3-ol (271 mg, 2.63 mmol), Cs₂CO₃ (1.28 g, 3.93 mmol) in toluene (50 mL) was added Pd₂(dba)₃ (240 mg, 0.262 mmol) and BINAP (326 mg, 0.524 mmol) at room temperature. Then the mixture was stirred at 120° C. for 16 h under N₂ and cooled to room temperature. The reaction mixture was filtered and the filter cake was washed with DCM (100 mL). The filtrate was concentrated in vacuo to give the crude product which was purified by column chromatography (eluent: PE:EA=0 to 1:1) to give 3-(((2-(((tert-butyldimethylsilyl)oxy)methyl)-6-cyclopropylimidazo[1,2-a]pyridin-8-yl)amino)methyl)oxetan-3-ol (210 mg, 39.8%) as a yellow oil. ESI-MS [M+H]+: 404.2.

### Synthesis of 7-(2-(((tert-butyldimethylsilyl)oxy)methyl)-6-cyclopropylimidazo[1,2-a]pyridin-8-yl)-2,5-dioxa-7-azaspiro[3.4]octan-6-one

To a solution of 3-(((2-(((tert-butyldimethylsilyl)oxy)methyl)-6-cyclopropylimidazo[1,2-a]pyridin-8-yl)amino)methyl)oxetan-3-ol (160 mg, 0.39 mmol) in ACN (20 mL) was added CDI (250 mg, 1.54 mmol) and the mixture was stirred at 80° C. for 5 h. After bringing the reaction to room temperature, water (50 mL) was added and the mixture was extracted with EtOAc (3×50 ml). The combined organics were washed with brine (50 mL), dried over Na₂SO₄ then concentrated in vacuo to give the crude product, which was purified by silica gel chromatography to give 7-(2-(((tert-butyldimethylsilyl)oxy)methyl)-6-cyclopropylimidazo[1,2-a]pyridin-8-yl)-2,5-dioxa-7-azaspiro[3.4]octan-6-one (76 mg, 44.7%) as a yellow oil. ESI-MS [M+H]+: 430.2.

### Synthesis of 7-(6-cyclopropyl-2-(hydroxymethyl)imidazo[1,2-a]pyridin-8-yl)-2,5-dioxa-7-azaspiro[3.4]octan-6-one

To a solution of 7-(2-(((tert-butyldimethylsilyl)oxy)methyl)-6-cyclopropylimidazo[1,2-a]pyridin-8-yl)-2,5-dioxa-7-azaspiro[3.4]octan-6-one (76 mg, 0.18 mmol) in THF (2 mL) was added TBAF (0.36 mL, 0.36 mmol) and the mixture was stirred at room temperature for 2 h. The reaction mixture was poured into water (30 mL) and extracted with EtOAc (3×30 ml). The combined organics were washed with brine (20 mL), dried over Na₂SO₄ then concentrated in vacuo to give the crude, which was purified by silica gel chromatography (eluant: EtOAc:PE from 0 to 50%) to give 7-(6-cyclopropyl-2-(hydroxymethyl)imidazo[1,2-a]pyridin-8-yl)-2,5-dioxa-7-azaspiro[3.4]octan-6-one (50 mg, 89%) as a white solid. ESI-MS [M+H]+: 316.1

### Synthesis of (R)-7-(2-(1-aminoethyl)-6-cyclopropylimidazo[1,2-a]pyridin-8-yl)-5-methyl-2-oxa-5,7-diazaspiro[3.4]octan-6-one

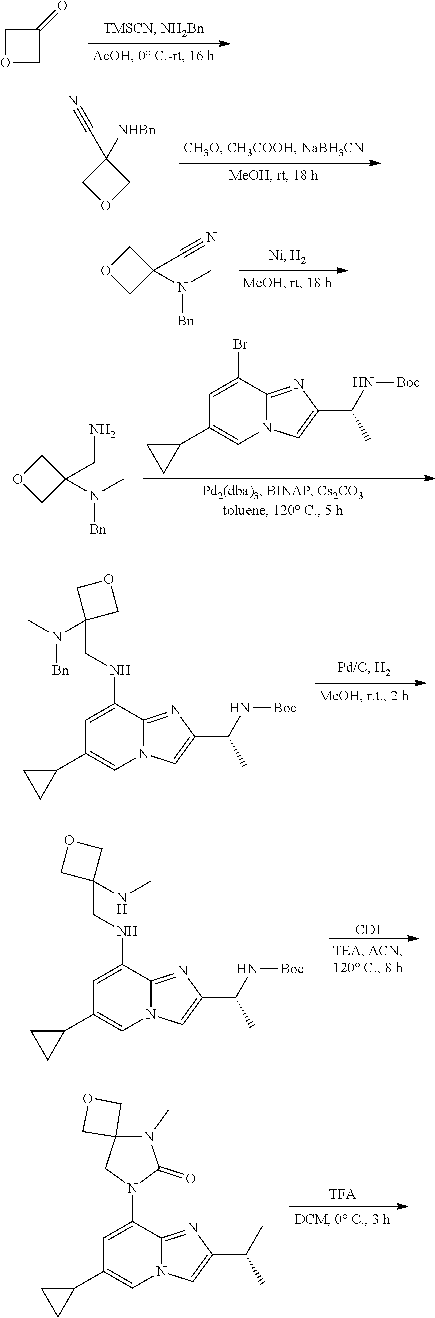

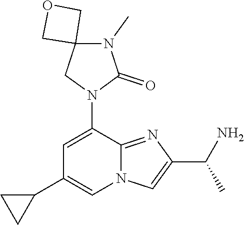 [SMILES: C[C@@H](N)c1cn2cc(C3CC3)cc(N3CC4(COC4)N(C)C3=O)c2n1]

### Synthesis of 3-(benzylamino)oxetane-3-carbonitrile

To a mixture of NH₂Bn (13 g, 0.12 mol) in AcOH (50 mL) was added oxetan-3-one (2.0 g, 28 mmol) and TMSCN (6.3 g, 64 mmol). The mixture was stirred at room temperature for 16 h. The mixture was concentrated to remove AcOH in vacuo, then diluted with saturated Na₂CO₃ (sat·, aq·, 100 mL) and extracted by EtOAc (100 mL×3). The combined organic layers were concentrated in vacuo and the residue was purified by column chromatography on silica gel, eluting with 0-20% EA/PE to afford 3-(benzylamino)oxetane-3-carbonitrile (3.7 g, 70%) as yellow oil. ESI-MS [M+H]+: 189.1.

### Synthesis of 3-(benzyl(methyl)amino)oxetane-3-carbonitrile

To a mixture of 3-(benzylamino)oxetane-3-carbonitrile (3.6 g, 19 mmol) in MeOH (60 mL) was added CH₂O (7.8 g, 0.26 mol) and CH₃COOH (0.5 mL). The mixture was stirred at room temperature for 16 h then NaBH₃CN (6.1 g, 96 mmol) was added. The mixture was stirred at room temperature for 2 h. Water (100 mL) was added and the mixture was extracted by DCM (100 mL×3). The combined organic layers were concentrated in vacuo and the residue was purified by flash column chromatography, eluting with 0-20% MeOH/DCM to afford 3-(benzyl(methyl)amino)oxetane-3-carbonitrile (0.50 g, 13%) as yellow oil. ESI-MS [M+H]+: 203.2.

### Synthesis of 3-(aminomethyl)-N-benzyl-N-methyloxetan-3-amine

To a mixture of 3-(benzyl(methyl)amino)oxetane-3-carbonitrile (0.50 g, 2.5 mmol) in MeOH (10 mL) was added Raney-Ni and stirred at room temperature for 16 h. The mixture was filtered through Celite® and the filter cake was washed with MeOH (30 mL). The filtrate was concentrated in vacuo and the residue was purified by column chromatography on silica gel, eluting with 0-20% MeOH/DCM to afford 3-(aminomethyl)-N-benzyl-N-methyloxetan-3-amine (0.24 g, 48%) as yellow oil. ESI-MS [M+H]+: 207.2.

### Synthesis of tert-butyl (R)-(1-(8-(((3-(benzyl(methyl)amino)oxetan-3-yl)methyl)amino)-6-cyclopropylimidazo[1,2-a]pyridin-2-yl)ethyl)carbamate

To a mixture of 3-(aminomethyl)-N-benzyl-N-methyloxetan-3-amine (0.14 g, 0.66 mmol), tert-butyl (R)-(1-(8-bromo-6-cyclopropylimidazo[1,2-a]pyridin-2-yl)ethyl)carbamate (0.15 g, 0.39 mmol), and Cs₂CO₃ (0.38 g, 1.2 mmol) in toluene (15 mL) were added Pd₂(dba)₃ (71 mg, 0.078 mmol) and BINAP (97 mg, 0.16 mmol). The reaction mixture was stirred at 120° C. for 5 h under N₂ and cooled to room temperature. The reaction mixture was filtered through Celite® and the filter cake was washed with DCM/MeOH (10/1, 40 mL). The filtrate was concentrated in vacuo to give the crude, which was purified by preparative TLC (eluent: EA/PE=2/1) to give tert-butyl (R)-(1-(8-(((3-(benzyl(methyl)amino)oxetan-3-yl)methyl)amino)-6-cyclopropylimidazo[1,2-a]pyridin-2-yl)ethyl)carbamate (0.16 g, 82%) as a yellow oil. ESI-MS [M+H]+: 506.3

### Synthesis of tert-butyl (R)-(1-(6-cyclopropyl-8-(((3-(methylamino)oxetan-3-yl)methyl)amino)imidazo[1,2-a]pyridin-2-yl)ethyl)carbamate

A mixture of tert-butyl (R)-(1-(8-(((3-(benzyl(methyl)amino)oxetan-3-yl)methyl)amino)-6-cyclopropylimidazo[1,2-a]pyridin-2-yl)ethyl)carbamate (316 mg, 0.62 mmol) and Pd/C (316 mg) in MeOH (10 mL) was stirred at room temperature for 2 h under H₂. After stirring at 90° C. for 16 h under N₂, the reaction mixture was filtered through Celite® and the filter cake was washed with DCM/MeOH (10/1, 100 mL). The filtrate was concentrated in vacuo to give the crude, which was purified by column chromatography (eluent: DCM/MeOH=50/1˜20/1) to give tert-butyl (R)-(1-(6-cyclopropyl-8-(((3-(methylamino)oxetan-3-yl)methyl)amino)imidazo[1,2-a]pyridin-2-yl)ethyl)carbamate (170 mg, 66%) as a yellow solid. ESI-MS [M+H]+: 416.2

### Synthesis of tert-butyl (R)-(1-(6-cyclopropyl-8-(5-methyl-6-oxo-2-oxa-5,7-diazaspiro[3.4]octan-7-yl)imidazo[1,2-a]pyridin-2-yl)ethyl)carbamate

A mixture of tert-butyl (R)-(1-(6-cyclopropyl-8-(((3-(methylamino)oxetan-3-yl)methyl)amino)imidazo[1,2-a]pyridin-2-yl)ethyl)carbamate (222 mg, 0.53 mmol), TEA (800 mg, 7.9 mmol) and CDI (700 mg, 4.3 mmol) in MeCN (6 mL) was stirred at 120° C. for 8 h. The reaction was quenched with water (10 mL) and extracted with EtOAc (3×20 mL). The combined organic layers were washed with brine (20 mL), dried over anhydrous Na₂SO₄ and concentrated in vacuo to give the crude product, which was purified by column chromatography (eluent: DCM/MeOH=20/1) to give tert-butyl (R)-(1-(6-cyclopropyl-8-(5-methyl-6-oxo-2-oxa-5,7-diazaspiro[3.4]octan-7-yl)imidazo[1,2-a]pyridin-2-yl)ethyl)carbamate (80 mg, 34%) as a white solid. ESI-MS [M+H]+: 442.2.

### Synthesis of (R)-7-(2-(1-aminoethyl)-6-cyclopropylimidazo[1,2-a]pyridin-8-yl)-5-methyl-2-oxa-5,7-diazaspiro[3.4]octan-6-one

To a solution of tert-butyl (R)-(1-(6-cyclopropyl-8-(5-methyl-6-oxo-2-oxa-5,7-diazaspiro[3.4]octan-7-yl)imidazo[1,2-a]pyridin-2-yl)ethyl)carbamate (40 mg, 0.09 mmol) in DCM (4 mL) was added TFA (0.4 mL). The resulting mixture was stirred at room temperature for 3 h. After cooled to 0° C., the mixture was quenched with a solution of NH₃ (7M in MeOH, 2 mL). The resulting solution was concentrated in vacuo to give the crude, which was purified by preparative TLC (eluent: DCM/MeOH=20/1) to give (R)-7-(2-(1-aminoethyl)-6-cyclopropylimidazo[1,2-a]pyridin-8-yl)-5-methyl-2-oxa-5,7-diazaspiro[3.4]octan-6-one (20 mg, 65%) as a yellow solid. ESI-MS [M+H]+: 342.1.

### Synthesis of (R)-7-(2-(1-aminoethyl)-6-cyclopropylimidazo[1,2-a]pyridin-8-yl)-2,5-dioxa-7-azaspiro[3.4]octan-6-one

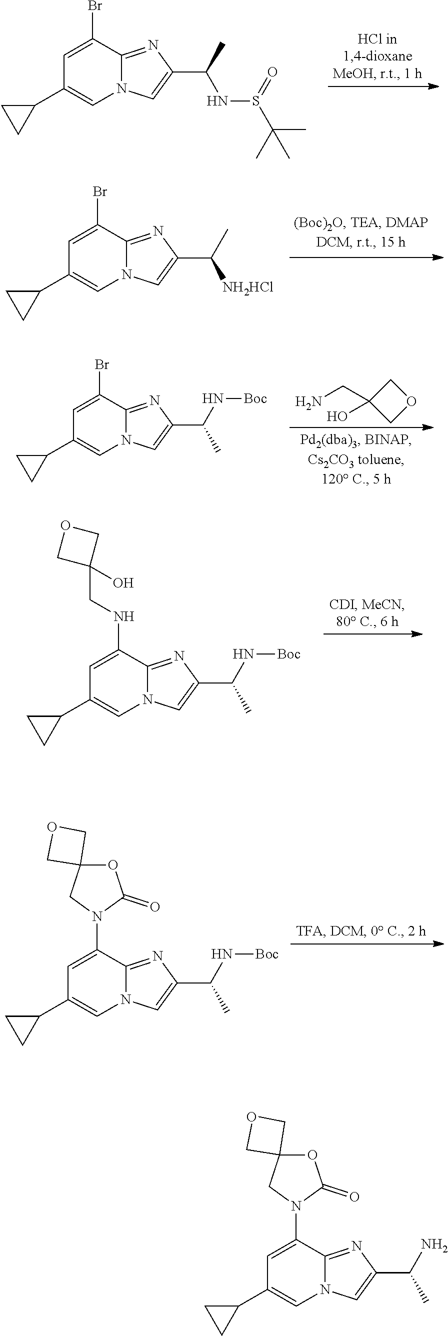

### Synthesis of (R)-1-(8-bromo-6-cyclopropylimidazo[1,2-a]pyridin-2-yl)ethan-1-amine hydrochloride

To a solution of N—((R)-1-(8-bromo-6-cyclopropylimidazo[1,2-a]pyridin-2-yl)ethyl)-2-methylpropane-2-sulfinamide (1.5 g, 3.9 mmol) in MeOH (10 mL) was added HCl (4M solution in 1,4-dioxane, 10 mL). The resulting mixture was stirred at room temperature for 1 h and concentrated in vacuo to give (R)-1-(8-bromo-6-cyclopropylimidazo[1,2-a]pyridin-2-yl)ethan-1-amine hydrochloride (1.3 g, 94%) as a yellow solid. ESI-MS: [M+H]⁺, 280.0

### Synthesis of tert-butyl (R)-(1-(8-bromo-6-cyclopropylimidazo[1,2-a]pyridin-2-yl)ethyl)carbamate

A mixture of (R)-1-(8-bromo-6-cyclopropylimidazo[1,2-a]pyridin-2-yl)ethan-1-amine hydrochloride (0.70 g, 2.5 mmol), DMAP (31 mg, 0.25 mmol), TEA (1.3 g, 13 mmol) and (Boc)₂O (0.83 g, 3.8 mmol) in DCM (10 mL) was stirred at room temperature for 15 h. The reaction was quenched with water (40 mL), extracted with DCM (3×40 mL). The combined organic layers were washed with brine (2×60 mL), dried over anhydrous Na₂SO₄, and concentrated in vacuo to give the crude, which was purified by silica gel chromatography (eluent: PE/EtOAc=3/2) to afford tert-butyl (R)-(1-(8-bromo-6-cyclopropylimidazo[1,2-a]pyridin-2-yl)ethyl)carbamate (0.60 g, 63%) as a yellow oil. ESI-MS: [M+H]⁺, 380.1

### Synthesis of tert-butyl (R)-(1-(6-cyclopropyl-8-(((3-hydroxyoxetan-3-yl)methyl)amino)imidazo[1,2-a]pyridin-2-yl)ethyl)carbamate

To a solution of tert-butyl (R)-(1-(8-bromo-6-cyclopropylimidazo[1,2-a]pyridin-2-yl)ethyl)carbamate (0.50 g, 1.3 mmol), 3-(aminomethyl)oxetan-3-ol (0.27 g, 2.6 mmol) and Cs₂CO₃ (1.3 g, 3.9 mmol) in toluene (50 mL) was added Pd₂(dba)₃ (0.24 g, 0.26 mmol) and BINAP (0.33 g, 0.52 mmol). After stirring at 120° C. for 5 h, the reaction mixture was filtered through Celite® and the filter cake was washed with DCM (50 mL×3). The filtrate was concentrated in vacuo to give the crude, which was purified by silica gel chromatography (eluent: MeOH/DCM=1/10) to afford tert-butyl (R)-(1-(6-cyclopropyl-8-(((3-hydroxyoxetan-3-yl)methyl)amino)imidazo[1,2-a]pyridin-2-yl)ethyl)carbamate (0.31 g, 58%) as a yellow solid. ESI-MS: [M+H]⁺, 403.2

### Synthesis of tert-butyl (R)-(1-(6-cyclopropyl-8-(6-oxo-2,5-dioxa-7-azaspiro[3.4]octan-7-yl)imidazo[1,2-a]pyridin-2-yl)ethyl)carbamate

A mixture of tert-butyl (R)-(1-(6-cyclopropyl-8-(((3-hydroxyoxetan-3-yl)methyl)amino)imidazo[1,2-a]pyridin-2-yl)ethyl)carbamate (0.31 g, 0.77 mmol) and 1,1′-carbonyldiimidazole (0.50 g, 3.1 mmol) in acetonitrile (40 mL) was stirred at 80° C. for 6 h. The reaction mixture was concentrated in vacuo to give the crude, which was purified by silica gel chromatography (eluent: MeOH/DCM=1/20) to afford tert-butyl (R)-(1-(6-cyclopropyl-8-(6-oxo-2,5-dioxa-7-azaspiro[3.4]octan-7-yl)imidazo[1,2-a]pyridin-2-yl)ethyl)carbamate (0.30 g, 91%) as a yellow solid. ESI-MS: [M+H]⁺, 429.2

### Synthesis of (R)-7-(2-(1-aminoethyl)-6-cyclopropylimidazo[1,2-a]pyridin-8-yl)-2,5-dioxa-7-azaspiro[3.4]octan-6-one

A mixture of tert-butyl (R)-(1-(6-cyclopropyl-8-(6-oxo-2,5-dioxa-7-azaspiro[3.4]octan-7-yl)imidazo[1,2-a]pyridin-2-yl)ethyl)carbamate (0.30 g, 0.70 mmol) and trifluoroacetic acid (1 mL) in DCM (10 mL) stirred at 0° C. for 2 h. The pH of reaction mixture was adjusted to 8 with NH₃ (7M solution in MeOH, 5 mL, 35 mmol) at 0° C. The mixture was diluted with DCM (40 mL) then concentrated in vacuo. The residue was dissolved in DCM (20 mL). The mixture was filtered and the filtrant washed by DCM (3×50 mL). The combined organics were concentrated in vacuo to give (R)-7-(2-(1-aminoethyl)-6-cyclopropylimidazo[1,2-a]pyridin-8-yl)-2,5-dioxa-7-azaspiro[3.4]octan-6-one (0.20 g, 87%) as a white solid. ESI-MS: [M+H]⁺, 329.2

### Synthesis of 2-(aminomethyl)-6-cyclopropyl-N,N-dimethylimidazo[1,2-a]pyridine-8-sulfonamide

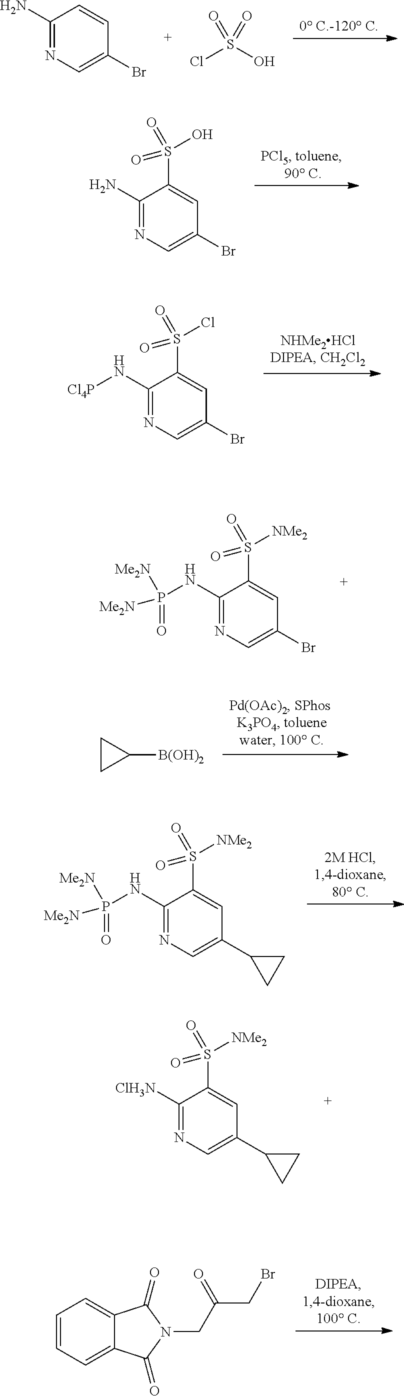

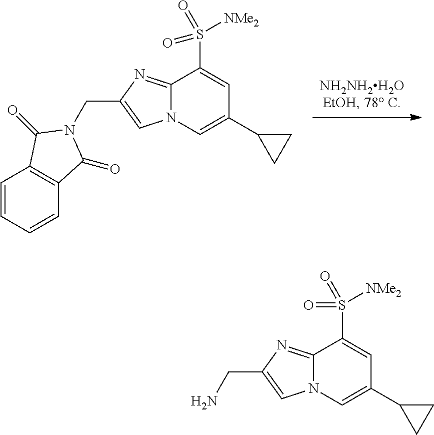 [SMILES: CN(C)S(=O)(=O)c1cc(C2CC2)cn2cc(CN3C(=O)c4ccccc4C3=O)nc12 ; "NH2NH2·H2OEtOH, 78° C." ; CN(C)S(=O)(=O)c1cc(C2CC2)cn2cc(CN)nc12]

### Synthesis of 2-amino-5-bromopyridine-3-sulfonic acid

5-bromopyridin-2-amine (1.29 g, 7.45 mmol), was added portionwise to stirred sulfurochloridic acid (5.0 mL) at 0° C. The reaction was then heated to 120° C. for 18 h. The reaction was cooled to room temperature and poured onto ice (250 g). The resulting precipitate was filtered, washed with water and dried to give the title product as a pale yellow powder (1.18 g, 58%). ¹H NMR (400 MHz, DMSO) δ 8.15-8.11 (m, 2H), 7.56 (s, 1H), 7.21 (d, J=9.3 Hz, 1H), 6.80 (d, J=8.3 Hz, 1H).

### Synthesis of 5-bromo-2-((tetrachloro-λ⁵-phosphanyl)amino)pyridine-3-sulfonyl chloride

2-amino-5-bromopyridine-3-sulfonic acid (1.0 g, 3.95 mmol) and PCl₅ (1.23 g, 5.93 mmol) were suspended in toluene (10.0 mL) and the reaction was heated at 90° C. for 18 h. The reaction was concentrated in vacuo and used in the next step without further purification.

### Synthesis of 2-((bis(dimethylamino)phosphoryl)amino)-5-bromo-N,N-dimethylpyridine-3-sulfonamide

DIPEA (6.9 mL, 39.50 mmol) was added to a stirred mixture of 5-bromo-2-((tetrachloro-λ⁵-phosphanyl)amino)pyridine-3-sulfonyl chloride (1.75 g, 3.95 mmol) and dimethylamine hydrochloride (3.22 g, 39.50 mmol) in DCM (40 mL) and stirred at room temperature for 18 h. The reaction was diluted with DCM (60 mL) and washed with water and brine. The combined organics were dried over MgSO₄ and concentrated in vacuo. The residue was used in the next step without further purification.

### Synthesis of 2-((bis(dimethylamino)phosphoryl)amino)-5-cyclopropyl-N,N-dimethylpyridine-3-sulfonamide

2-((bis(dimethylamino)phosphoryl)amino)-5-bromo-N,N-dimethylpyridine-3-sulfonamide (1.64 g, 3.95 mmol), cyclopropylboronic acid (0.51 g, 5.93 mmol), Pd(OAc)₂ (44 mg, 0.20 mmol), SPhos (160 mg, 0.40 mmol) and K₃PO₄ (2.93 g, 13.83 mmol) were suspended in toluene (20 mL) and water (2.0 mL), the reaction was degassed with N₂ for 5 min. The reaction was then heated to 100° C. for 2.5 h. The reaction was cooled to room temperature, diluted with EtOAc (60 mL) and washed with water and brine. The combined organics were dried over MgSO₄ and concentrated in vacuo. The residue was purified by silica gel chromatography, eluting with a gradient of 0-6% MeOH in DCM to give the title compound (805 mg, 54%) which was used without further purification.

### Synthesis of 2-amino-5-cyclopropyl-N,N-dimethylpyridine-3-sulfonamide hydrochloride

2-((bis(dimethylamino)phosphoryl)amino)-5-cyclopropyl-N,N-dimethylpyridine-3-sulfonamide (805 mg, 2.14 mmol), was suspended HCl (2M aq·, 4.3 mL, 8.58 mmol) and 1,4-dioxane (15.0 mL) and heated at 80° C. for 48 h. The mixture was then cooled to room temperature and concentrated in vacuo. The residue was dissolved in DCM (100 mL) and washed with NaHCO₃ (sat·aq·, 2×40 mL) and brine (50 mL), the combined organics were dried over MgSO₄ and concentrated in vacuo to give the title compound (430 mg, 83%). ¹H NMR (400 MHz, DMSO) δ 8.13 (d, J=2.3 Hz, 1H), 7.47 (d, J=2.5 Hz, 1H), 6.50 (s, 2H), 2.72 (s, 6H), 1.98-1.90 (m, 1H), 0.94 (ddd, J=4.4, 6.3, 8.5 Hz, 2H), 0.69-0.64 (m, 2H).

### Synthesis of 6-cyclopropyl-2-((1,3-dioxoisoindolin-2-yl)methyl)-N,N-dimethylimidazo[1,2-a]pyridine-8-sulfonamide

DIPEA (0.23 mL, 1.30 mmol) was added to a stirred solution of 2-amino-5-cyclopropyl-N,N-dimethylpyridine-3-sulfonamide hydrochloride (360 mg, 0.648 mmol) and 2-(3-bromo-2-oxopropyl)isoindoline-1,3-dione (200 mg, 0.713 mmol) in dioxane (10.0 mL). The reaction was heated at 100° C. for 18 h. The reaction was cooled to room temperature and then concentrated in vacuo. The residue was dissolved in EtOAc (100 mL) and washed with NaHCO₃ (sat·aq·, 2×40 mL) and brine (50 mL), the combined organics were dried over MgSO₄ and concentrated in vacuo. The residue was purified by silica gel chromatography, eluting with a gradient of 0-100% DCM in EtOAc to give the title compound (270 mg, 95%). ESI-MS (M+H)+: 425, ¹H NMR (400 MHz, DMSO) δ 8.57 (d, J=1.0 Hz, 1H), 7.99-7.91 (m, 5H), 7.53 (d, J=1.5 Hz, 1H), 4.99 (s, 2H), 2.74 (s, 6H), 2.13-2.05 (m, 1H), 1.01 (ddd, J=4.4, 6.4, 8.3 Hz, 2H), 0.78-0.72 (m, 2H).

### Synthesis of 2-(aminomethyl)-6-cyclopropyl-N,N-dimethylimidazo[1,2-a]pyridine-8-sulfonamide

Using a similar procedure to that for 3-(2-(aminomethyl)-6-cyclopropylimidazo[1,2-a]pyridin-8-yl)oxetan-3-ol, 2-(aminomethyl)-6-cyclopropyl-N,N-dimethylimidazo[1,2-a]pyridine-8-sulfonamide (220 mg at 79% purity) was synthesised from 6-cyclopropyl-2-((1,3-dioxoisoindolin-2-yl)methyl)-N,N-dimethylimidazo[1,2-a]pyridine-8-sulfonamide (285 mg, 0.671 mmol) using hydrazine hydrate. ESI-MS (M+H)+: 295.3, ¹H NMR (400 MHz, DMSO) δ 8.63 (d, J=1.0 Hz, 1H), 7.86 (s, 1H), 7.51 (d, J=1.8 Hz, 1H), 3.88 (s, 2H), 2.91 (s, 6H), 2.14-2.06 (m, 1H), 1.05-1.00 (m, 2H), 0.80-0.74 (m, 2H).

### Synthesis of 2-(2-(aminomethyl)-6-cyclopropylimidazo[1,2-a]pyridin-8-yl)propan-2-ol

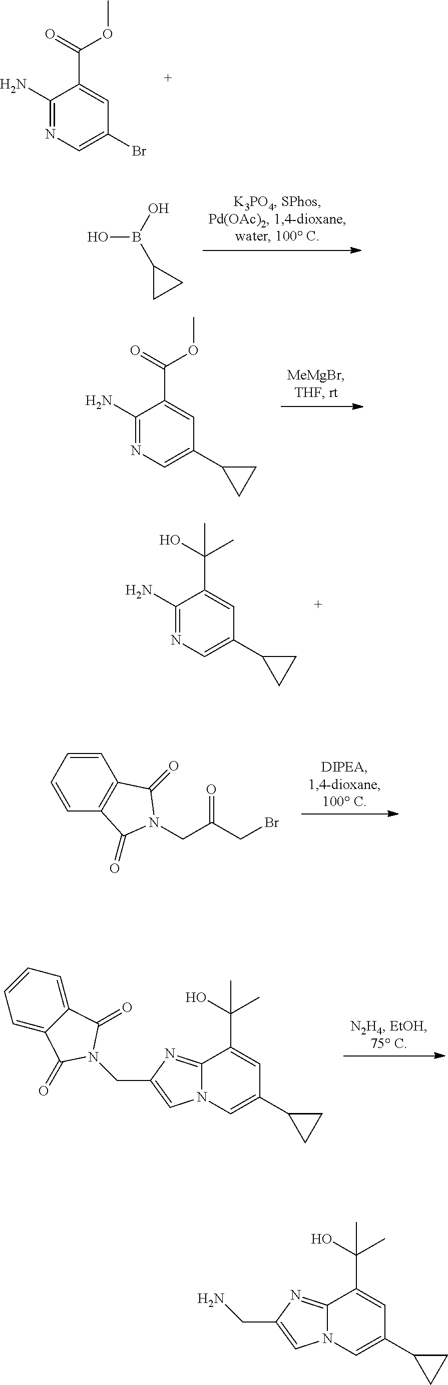

### Synthesis of methyl 2-amino-5-cyclopropylnicotinate

Methyl 2-amino-5-bromonicotinate (2.0 g, 8.7 mmol), cyclopropylboronic acid (1900 mg, 22 mmol), SPhos (360 mg, 0.87 mmol) and K₃PO₄ (6.4 g, 30 mmol) were suspended in 1,4-dioxane (40 mL) and water (4.0 mL) and degassed for 5 min. Pd(OAc)₂ (97 mg, 0.4 mmol) was added and the reaction mixture was stirred at 100° C. for 16 h. The mixture was cooled to room temperature, diluted with EtOAc (100 mL) and washed with water (70 mL) and brine (3×50 mL). The organics were dried over MgSO₄ and concentrated in vacuo. The residue was purified by silica gel chromatography eluting with a gradient of 0-100% EtOAc in isohexane to give the title compound (1.2 g, 72%).

ESI-MS (M+H)+: 193.1, ¹H NMR (400 MHz, CDCl₃) δ 8.09 (d, J=2.4 Hz, 1H), 7.83-7.81 (m, 1H), 6.32-6.32 (m, 2H), 3.88 (s, 3H), 1.83-1.77 (m, 1H), 0.93-0.88 (m, 2H), 0.64-0.56 (m, 2H).

### Synthesis of 2-(2-Amino-5-cyclopropylpyridin-3-yl)propan-2-ol

A solution of methyl 2-amino-5-cyclopropylnicotinate (1200 mg, 6.2 mmol) in THF (30 mL) was cooled to −20° C. and methylmagnesium bromide (3M in Et₂O, 13 mL, 38 mmol) was added dropwise. The reaction mixture was allowed to warm up to room temperature over 16 h. The mixture was quenched with sodium bicarbonate (sat·aq·, 50 mL), diluted with EtOAc (70 mL), filtered and separated. The organics were washed with brine (50 mL), dried over MgSO₄ and concentrated in vacuo. The residue was purified by silica gel chromatography eluting with a gradient of 0-100% EtOAc in cyclohexane to give the title compound (750 mg, 63%).

ESI-MS (M+H)+: 193.2, ¹H NMR (400 MHz, DMSO) δ 7.65 (d, J=2.1 Hz, 1H), 7.00 (d, J=2.1 Hz, 1H), 5.84 (s, 2H), 5.32 (s, 1H), 1.81-1.73 (m, 1H), 1.48 (s, 6H), 0.85-0.80 (m, 2H), 0.57-0.52 (m, 2H).

### Synthesis of 2-((6-cyclopropyl-8-(2-hydroxypropan-2-yl)imidazo[1,2-a]pyridin-2-yl)methyl)isoindoline-1,3-dione

A solution of 2-(2-amino-5-cyclopropylpyridin-3-yl)propan-2-ol (700 mg, 3.6 mmol), 2-(3-bromo-2-oxopropyl)isoindoline-1,3-dione (1300 mg, 4.0 mmol) and DIPEA (0.95 mL, 5.5 mmol) in 1,4-dioxane (80 mL) was stirred at 100° C. for 16 h. The mixture was cooled to room temperature, diluted with EtOAc (150 mL) and washed with sodium bicarbonate (2×70 mL). The organics were dried over MgSO₄ and concentrated in vacuo. The residue was purified by silica gel chromatography eluting with a gradient of 0-100% EtOAc in cyclohexane to give the title compound (800 mg, 59%). ESI-MS (M+H)+: 376.2, ¹H NMR (400 MHz, CDCl₃) δ 7.87-7.85 (m, 2H), 7.73-7.70 (m, 2H), 7.67 (s, 1H), 7.42 (s, 1H), 6.78 (d, J=1.6 Hz, 1H), 6.38 (s, 1H), 5.02 (s, 2H), 1.89-1.81 (m, 1H), 1.64 (s, 6H), 0.96-0.90 (m, 2H), 0.64-0.59 (m, 2H).

### Synthesis of 2-(2-(aminomethyl)-6-cyclopropylimidazo[1,2-a]pyridin-8-yl)propan-2-ol

Using a similar procedure to that for 3-(2-(aminomethyl)-6-cyclopropylimidazo[1,2-a]pyridin-8-yl)oxetan-3-ol, 2-(2-(aminomethyl)-6-cyclopropylimidazo[1,2-a]pyridin-8-yl)propan-2-ol (500 mg, 96%) was synthesised from 2-((6-cyclopropyl-8-(2-hydroxypropan-2-yl)imidazo[1,2-a]pyridin-2-yl)methyl)isoindoline-1,3-dione (800 mg, 2.13 mmol) using hydrazine hydrate.

ESI-MS (M+H)+: 246.2, ¹H NMR (400 MHz, CDCl₃) δ 7.74-7.73 (m, 1H), 7.34 (s, 1H), 6.79 (d, J=1.6 Hz, 1H), 3.97 (s, 2H), 1.92-1.83 (m, 1H), 1.69 (s, 6H), 0.98-0.92 (m, 2H), 0.68-0.63 (m, 2H). OH and NH₂ not observed

### Synthesis of (R)-1-(2-(1-aminoethyl)-6-(1-fluorocyclopropyl)imidazo[1,2-a]pyridin-8-yl)-3-methylimidazolidine-2,4-dione

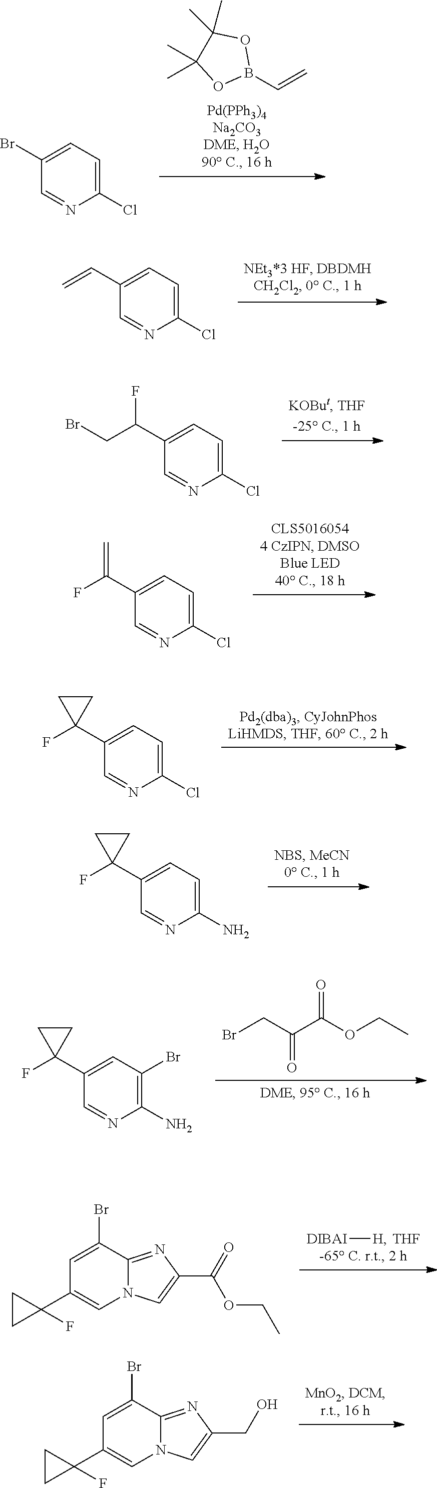

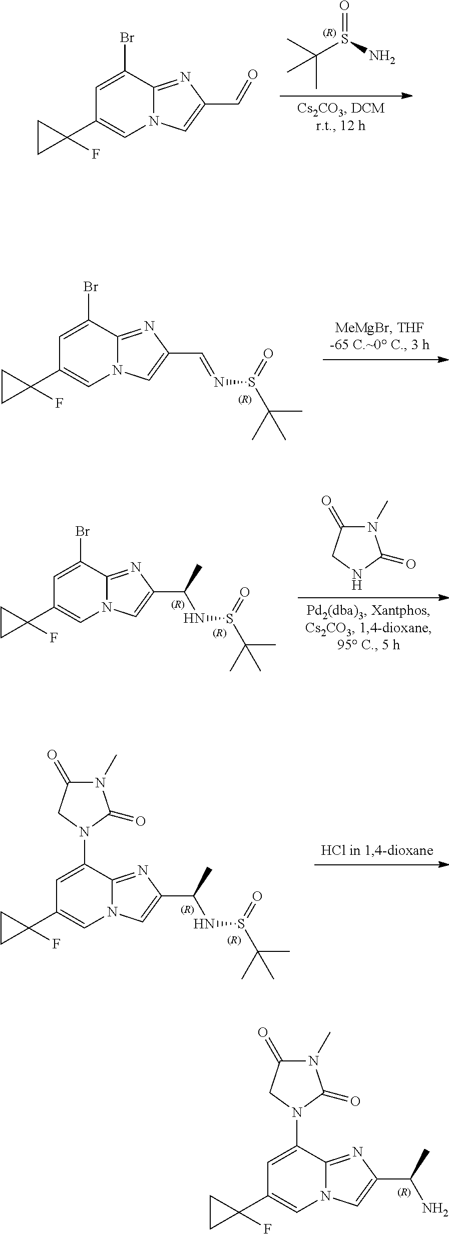

### Synthesis of 2-chloro-5-vinylpyridine

A mixture of 5-bromo-2-chloropyridine (10.0 g, 52.0 mmol), vinyl boronic acid pinacol ester (10.4 g, 67.6 mmol), 1,2-dimethoxyethane (250 mL) and Na₂CO₃ (2 M aq·, 78 mL) was degassed (by vacuum/nitrogen refill cycles). Pd(PPh₃)₄ (3.00 g, 2.60 mmol) was added and the mixture was heated at 90° C. for 16 h. The mixture was cooled to room temperature, diluted with ethyl acetate (250 mL) and Na₂CO₃ (10% aq·, 150 mL). The layers were separated, and the organic layer was washed with brine, dried with Na₂SO₄, filtered and concentrated in vacuo to obtain a golden oil, which was loaded onto a silica gel column (250 g) equilibrated with heptane. Elution with heptane:ethyl acetate (1:0-96:4) afforded 2-chloro-5-vinylpyridine (4.71 g, 65%), as colourless liquid. ESI-MS [M+H]+: 140/142. 1H NMR (400 MHz, CDCl₃, ppm) δ 8.37 (d, J 3 Hz, 1H), 7.70 (dd, J 9, 3 Hz, 1H), 7.29 (d, J 9 Hz, 1H), 6.67 (dd, J 18, 11 Hz, 1H), 5.81 (d, J 18 Hz, 1H), 5.42 (d, J 11 Hz, 1H).

### Synthesis of 5-(2-bromo-1-fluoroethyl)-2-chloropyridine

A solution of 2-chloro-5-vinylpyridine (4.71 g, 33.7 mmol) in dichloromethane (45 mL) was added to a suspension of 1,3-dibromo-5,5-dimethylhydantoin (14.5 g, 50.6 mmol) in dichloromethane (40 mL) at 0° C. After being stirred for 30 min the mixture was treated with triethylamine trihydrofluoride (8.24 mL, 50.6 mmol). After a further 30 min the reaction was quenched by being poured into NaHCO₃ (sat·aq·, 250 mL). After being stirred for 30 min the mixture was diluted with ethyl acetate (250 mL). The layers were separated, and the organic layer washed with NaHSO3 (5%, aq·, 100 mL), followed by NaHCO₃ (sat·aq·, 100 mL). The organic solution was washed with brine, dried with Na₂SO₄, filtered and concentrated to obtain an orange oil, which was purified on silica gel (200 g) column purification using a gradient of heptane:ethyl acetate (95:5 to 8:2) to afford 5-(2-bromo-1-fluoroethyl)-2-chloropyridine (4.38 g, 54%), as a pale yellow oil. ESI-MS [M+H]+: 238/240/242. 1H NMR (400 MHz, CDCl₃, ppm) δ 8.41 (s, 1H), 7.70 (dd, J 8, 2 Hz, 1H), 7.40 (d, J 8 Hz, 1H), 5.68 (dt, J 46, 6 Hz, 1H), 3.75-3.59 (m, 2H). 19F NMR (373 MHz, CDCl₃, ppm) δ −175.1 (dt, J 46, 18 Hz, 1F).

### Synthesis of 2-chloro-5-(1-fluorovinyl)pyridine

A solution of potassium tert-butoxide (1 M in tetrahydrofuran, 29.4 ml, 29.4 mmol) was added via syringe pump over 60 min to a rapidly stirred solution of 5-(2-bromo-1-fluoroethyl)-2-chloropyridine in anhydrous tetrahydrofuran (100 mL) at −25° C. After the addition was complete, the mixture was poured into citric acid (1 M aq·, 50 mL). The pH was raised to ˜7 by addition of NaHCO₃ (sat·aq·, 150 mL). The mixture was diluted with methyl tert-butyl ether (300 mL) and the layers were separated. The organic layer was washed with brine (2×150 mL), dried (MgSO₄), filtered and concentrated in vacuo to obtain a light brown oil. The crude product was purified on silica gel (100 g) using a gradient of heptane:ethyl acetate (1:0-9:1) to afford 2-chloro-5-(1-fluorovinyl)pyridine (2.46 g, 85%), as a colourless oil. ESI-MS [M+H]+ 158/160, 1H NMR (400 MHz, CDCl₃, ppm) δ 8.59 (d, J 2 Hz, 1H), 7.78 (dd, J 9, 2 Hz, 1H), 7.36 (d, J 9 Hz, 1H), 5.11 (dd, J 49, 4 Hz, 1H), 5.02 (dd, J 18, 4 Hz, 1H). 19F NMR (376 MHz, CDCl₃, ppm) δ −108.7 (dd, J 49, 18 Hz, 1F).

### Synthesis of 2-chloro-5-(1-fluorocyclopropyl)pyridine

An oven-dried 250 mL round bottom flask equipped with a magnetic stir bar was charged with 2-chloro-5-(1-fluorovinyl)pyridine (1.53 g, 9.74 mmol), 4CzIPN (384 mg, 0.49 mmol), triethylammonium bis(catecholato)iodomethylsilicate (7.12 g, 14.6 mmol) and anhydrous DMSO (100 mL). The resulting solution was degassed (vacuum/nitrogen refill×3) and placed in front of two blue LED lamps (Kessil, H150 Growlights, 32W, 420-500 nm). The lamps were arranged on both sides of the flask as close as possible to its surface. A fan for cooling was mounted over the reaction flask. The reaction was left for 18 h. Proton NMR of a sample showed complete consumption of starting material. The heterogeneous mixture was poured into methyl tert-butyl ether (250 mL). NaHCO₃ (Sat·, aq·, 150 mL) and water (300 mL) were added with stirring. After for 10 min Dicalite (30 g) was added and the mixture was stirred for another 10 min. The mixture was filtered through a sinter funnel, washing the precipitate with additional methyl tert-butyl ether (2×200 mL). The clear biphasic filtrate was transferred to a separatory funnel and the layers were separated. The organic layer was washed with more saturated aqueous sodium bicarbonate (250 mL). The combined aqueous layers were extracted with methyl tert-butyl ether (100 mL). The organic solutions were combined, washed with brine (2×150 mL), dried (Na₂SO₄), filtered and concentrated under reduced pressure to obtain a dark yellow gum. Purification by flash chromatography on silica gel (150 g), eluting with pentane:methyl tert-butyl ether=96:4 (500 mL), 94:6 (500 mL) and 92:8 (1 L), gave 2-chloro-5-(1-fluorocyclopropyl)pyridine (673 mg, 40%), as a yellow oil that crystallised upon standing. ESI-MS [M+H]+ 172/174, 1H NMR (400 MHz, CDCl₃, ppm) δ 8.28 (d, J 2 Hz, 1H), 7.56 (dd, J 8, 2 Hz, 1H), 7.33 (d, J 8 Hz, 1H), 1.62-1.52 (m, 2H), 1.12-1.05 (m, 2H).

### Synthesis of 5-(1-fluorocyclopropyl)pyridin-2-amine

To a stirred solution of 2-chloro-5-(1-fluorocyclopropyl)pyridine (860 mg, 5 mmol), Pd₂(dba)₃ (458 mg, 0.5 mmol) and CyJohnPhos (350 mg, 1 mmol) in THF (50 mL) was added LiHMDS (25 ml, 25 mmol. After stirring at 60° C. for 2 h under N₂, the reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by silica gel chromatography (eluent: DCM/MeOH=30/1) to give the 5-(1-fluorocyclopropyl)pyridin-2-amine (450 mg, 59%) as a yellow solid. ESI-MS [M+H]⁺: 153.1.

### Synthesis of 3-bromo-5-(1-fluorocyclopropyl)pyridin-2-amine

To a stirred solution of 5-(1-fluorocyclopropyl)pyridin-2-amine (450 mg, 3 mmol) in MeCN (20 mL) was added a solution of NBS (587 mg, 3.3 mmol) in MeCN (5 mL) at 0° C. After stirring at 0° C. for 0.5 h. water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent: EA/PE=1/5) to give the 3-bromo-5-(1-fluorocyclopropyl)pyridin-2-amine (290 mg, 42%) as a yellow solid. ESI-MS [M+H]⁺: 231.0.

### Synthesis of ethyl 8-bromo-6-(1-fluorocyclopropyl)imidazo[1,2-a]pyridine-2-carboxylate

To a mixture of 3-bromo-5-(1-fluorocyclopropyl)pyridin-2-amine (290 mg, 1.3 mmol) in DME (20 mL) was added ethyl 3-bromo-2-oxopropanoate (761 mg, 3.9 mmol) and the mixture was stirred at 95° C. for 16 h. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give the crude, which was purified by silica gel chromatography (eluent: PE/EtOAc=5/1) to give ethyl 8-bromo-6-(1-fluorocyclopropyl)imidazo[1,2-a]pyridine-2-carboxylate (260 mg, 63%) as a yellow solid. ESI-MS [M+H]⁺: 327.0.

### Synthesis of (8-bromo-6-(1-fluorocyclopropyl)imidazo[1,2-a]pyridin-2-yl)methanol

To a stirred solution of ethyl 8-bromo-6-(1-fluorocyclopropyl)imidazo[1,2-a]pyridine-2-carboxylate (260 mg, 0.8 mmol) in THF (10 mL) was slowly added DIBAL-H (2.4 mmol, 2.4 mL) at −65° C. Then the reaction was stirred at −65° C. for 2 h and warmed to room temperature. The reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by silica gel chromatography (eluent: EA/PE=1/3) to give the (8-bromo-6-(1-fluorocyclopropyl)imidazo[1,2-a]pyridin-2-yl)methanol (160 mg, 71%) as yellow solid. ESI-MS [M+H]+: 285.1.

### Synthesis of 8-bromo-6-(1-fluorocyclopropyl)imidazo[1,2-a]pyridine-2-carbaldehyde

To a mixture of (8-bromo-6-(1-fluorocyclopropyl)imidazo[1,2-a]pyridin-2-yl)methanol (158 mg, 0.55 mmol) in DCM (20 mL) was added MnO₂ (479 mg, 5.5 mmol). The resulting mixture was stirred at room temperature for 16 h, then concentrated in vacuo to give the crude, which was purified by silica gel chromatography (eluent: EA/PE=1/3) to give the 8-bromo-6-(1-fluorocyclopropyl)imidazo[1,2-a]pyridine-2-carbaldehyde (142 mg, 92%) as a yellow solid. ESI-MS [M+H]+: 283.0.

### Synthesis of (R, E)-N-((8-bromo-6-(1-fluorocyclopropyl)imidazo[1,2-a]pyridin-2-yl)methylene)-2-methylpropane-2-sulfinamide

To a mixture of 8-bromo-6-(1-fluorocyclopropyl)imidazo[1,2-a]pyridine-2-carbaldehyde (142 mg, 0.5 mmol) and (R)-2-methylpropane-2-sulfinamide (91 mg, 0.75 mmol) in DCM (15 mL) was added Cs₂CO₃ (326 mg, 1.0 mmol). The reaction was stirred at room temperature for 16 h, then concentrated in vacuo to give the crude, which was purified by silica gel chromatography (eluent: EA/PE=1:1) to give (R, E)-N-((8-bromo-6-(1-fluorocyclopropyl)imidazo[1,2-a]pyridin-2-yl)methylene)-2-methylpropane-2-sulfinamide (160 mg, 83%) as yellow solid. ESI-MS [M+H]+: 386.0.

### Synthesis of (R)—N—((R)-1-(8-bromo-6-(1-fluorocyclopropyl)imidazo[1,2-a]pyridin-2-yl)ethyl)-2-methylpropane-2-sulfinamide

To a solution of (R, E)-N-((8-bromo-6-(1-fluorocyclopropyl)imidazo[1,2-a]pyridin-2-yl)methylene)-2-methylpropane-2-sulfinamide (160 mg, 0.41 mmol) in THF (10 mL) was slowly added methylmagnesium bromide (1.2 mmol, 1M in THF, 1.2 mL) at −65° C. Then the reaction was stirred at −65° C. for 3 h under N₂. The reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by silica gel chromatography (eluent: EA/PE=1/1) to give the (R)—N—((R)-1-(8-bromo-6-(1-fluorocyclopropyl)imidazo[1,2-a]pyridin-2-yl)ethyl)-2-methylpropane-2-sulfinamide (120 mg, 72%) as a yellow solid. ESI-MS [M+H]+: 402.1.

### Synthesis of (R)—N—((R)-1-(6-(1-fluorocyclopropyl)-8-(3-methyl-2,4-dioxoimidazolidin-1-yl)imidazo[1,2-a]pyridin-2-yl)ethyl)-2-methylpropane-2-sulfinamide

To a solution of (R)—N—((R)-1-(8-bromo-6-(1-fluorocyclopropyl)imidazo[1,2-a]pyridin-2-yl)ethyl)-2-methylpropane-2-sulfinamide (100 mg, 0.25 mmol), 3-methylimidazolidine-2,4-dione (86 mg, 0.75 mmol) and Cs₂CO₃ (245 mg, 0.75 mmol) in 1,4-dioxane (20 mL) was added Pd₂(dba)₃ (23 mg, 0.025 mmol) and Xantphos (29 mg, 0.05 mmol). The reaction mixture was filtered through Celite® and the filter cake was washed with DCM/MeOH (10/1, 100 mL). The filtrate was concentrated in vacuo to give the crude, which was purified by silica gel chromatography (eluent: MeOH/DCM=1/30) to give the (R)—N—((R)-1-(6-(1-fluorocyclopropyl)-8-(3-methyl-2,4-dioxoimidazolidin-1-yl)imidazo[1,2-a]pyridin-2-yl)ethyl)-2-methylpropane-2-sulfinamide (90 mg, 83%) as a yellow solid. ESI-MS [M+H]+: 436.2.

### Synthesis of (R)-1-(2-(1-aminoethyl)-6-(1-fluorocyclopropyl)imidazo[1,2-a]pyridin-8-yl)-3-methylimidazolidine-2,4-dione

To a mixture of (R)—N—((R)-1-(6-(1-fluorocyclopropyl)-8-(3-methyl-2,4-dioxoimidazolidin-1-yl)imidazo[1,2-a]pyridin-2-yl)ethyl)-2-methylpropane-2-sulfinamide (90 mg, 0.21 mol) in 1,4-dioxane (2 mL) was added HCl (4M solution in dioxane, 2 mL). After stirring at room temperature for 1 h, the resulting reaction solution was neutralized with saturated aq·NaHCO₃ solution and extracted with DCM (3×20 mL). The combined organic layers were washed with brine, dried over Na₂SO₄, concentrated in vacuo to give the crude, which was purified by preparative TLC to give (R)-1-(2-(1-aminoethyl)-6-(1-fluorocyclopropyl)imidazo[1,2-a]pyridin-8-yl)-3-methylimidazolidine-2,4-dione (42 mg, 61%) as a yellow solid. ESI-MS [M+H]+: 332.2.

### Synthesis of 3-(2-(aminomethyl)-6-cyclopropylimidazo[1,2-a]pyridin-8-yl)propanenitrile

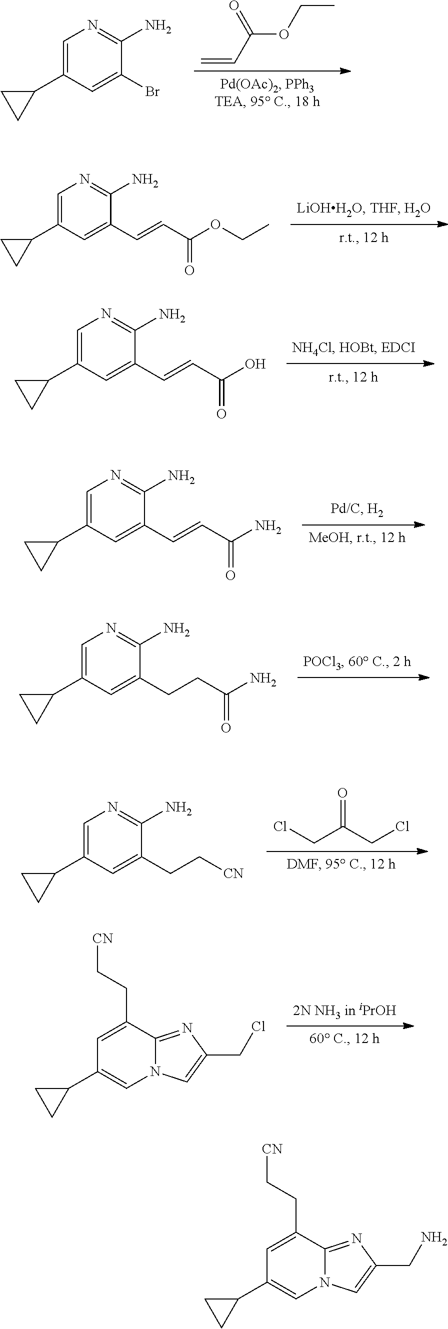

### Synthesis of ethyl (E)-3-(2-amino-5-cyclopropylpyridin-3-yl)acrylate

A mixture of 3-bromo-5-cyclopropylpyridin-2-amine (1.5 g, 7.0 mmol), Pd(OAc)₂ (0.16 g, 0.71 mmol), PPh₃ (0.93 g, 3.5 mmol), ethyl acrylate (1.4 g, 14 mmol) and Et₃N (2.1 g, 21 mmol) in DMF (30 mL) was stirred at 95° C. under N₂ for 16 h. After cooling to room temperature, the reaction mixture was quenched with water (40 mL) and extracted with EtOAc (6×70 mL). The combined organic layers were washed with saturated brine solution (50 mL), dried over Na₂SO₄, and concentrated in vacuo to give the crude, which was purified with column chromatography on silica gel (eluent: PE/EtOAc=1/1) to give ethyl (E)-3-(2-amino-5-cyclopropylpyridin-3-yl)acrylate (0.70 g, 43%). as a yellow solid. ESI-MS [M+H]+: 233.1

### Synthesis of (E)-3-(2-amino-5-cyclopropylpyridin-3-yl)acrylic acid

A mixture of ethyl (E)-3-(2-amino-5-cyclopropylpyridin-3-yl)acrylate (0.70 g, 3.0 mmol) and LiOH—H₂O (0.51 g, 12 mmol) in THF (30 mL) and H₂O (10 mL) was stirred at room temperature for 12 h. The reaction mixture was concentrated in vacuo to remove the THF and pH was adjusted to 3˜4 with HCl (4 N in dioxane). The resulting mixture was concentrated in vacuo to give crude (E)-3-(2-amino-5-cyclopropylpyridin-3-yl)acrylic acid (0.75 g, quant), which was used to the next step directly without further purification. ESI-MS [M+H]+: 205.2

### Synthesis of (E)-3-(2-amino-5-cyclopropylpyridin-3-yl)acrylamide

A mixture of (E)-3-(2-amino-5-cyclopropylpyridin-3-yl)acrylic acid (0.75 g, 3.7 mmol), (NH₄)₂CO₃ (1.8 g, 19 mmol), HOBt (0.99 g, 7.3 mmol), EDCI (1.4 g, 7.3 mmol), and DIPEA (2.4 g, 19 mmol) in DMF (25 mL) was stirred at room temperature for 12 h. The reaction mixture was quenched with water (20 mL) and extracted with EtOAc (8×50 mL). The combined organic layers were washed with saturated brine solution (50 mL), dried over Na₂SO₄, and concentrated in vacuo to give the crude, which was purified with column chromatography on silica gel (eluent: DCM/CH₃OH=10/1) to give (E)-3-(2-amino-5-cyclopropylpyridin-3-yl)acrylamide (0.51 g, 68%) as a white solid. ESI-MS [M+H]+: 204.2

### Synthesis of 3-(2-amino-5-cyclopropylpyridin-3-yl)propanamide

A mixture of (E)-3-(2-amino-5-cyclopropylpyridin-3-yl)acrylamide (0.51 g, 2.5 mmol) in MeOH (30 mL) and Pd/C (80 mg) was stirred at room temperature under H₂ for 12 h. The mixture was filtered and the filtrate was concentrated in vacuo to give the crude, which was purified with column chromatography on silica gel (eluent: DCM/CH₃OH=10/1) to give 3-(2-amino-5-cyclopropylpyridin-3-yl)propanamide (0.31 g, 60%) as a yellow solid. ESI-MS [M+H]+: 205.1

### Synthesis of 3-(2-amino-5-cyclopropylpyridin-3-yl)propanenitrile

A mixture of 3-(2-amino-5-cyclopropylpyridin-3-yl)propanamide (0.31 g, 1.5 mmol) in POCl₃ (15 mL) was stirred at 60° C. under N₂ for 2 h. After cooling to room temperature, the reaction solution was concentrated in vacuo and the pH adjusted to 7˜8 with saturated aq·NaHCO₃. The resulting mixture was extracted with EtOAc (3×30 mL). The combined organic layers were washed with saturated brine solution (30 mL), dried over Na₂SO₄ and concentrated in vacuo to give the crude, which was purified with preparative TLC (eluent: DCM/CH₃OH=10/1) to give 3-(2-amino-5-cyclopropylpyridin-3-yl)propanenitrile (0.12 g, 43%) as a white solid. ESI-MS [M+H]+: 187.2

### Synthesis of 3-(2-(chloromethyl)-6-cyclopropylimidazo[1,2-a]pyridin-8-yl)propanenitrile

To a solution of 3-(2-amino-5-cyclopropylpyridin-3-yl)propanenitrile (0.12 g, 0.64 mmol) in DMF (15 mL) was added 1,3-dichloropropan-2-one (0.24 g, 1.9 mmol). The resulting mixture was stirred at 95° C. under N₂ for 13 h. After cooling to room temperature, the reaction was quenched with water (20 mL) and extracted with EtOAc (3×30 mL). The combined organic layers were washed with saturated brine solution (20 mL), dried over Na₂SO₄, and concentrated in vacuo to give the crude, which was purified with preparative TLC (eluent: PE/EA=1/1) to give the 3-(2-(chloromethyl)-6-cyclopropylimidazo[1,2-a]pyridin-8-yl)propanenitrile (70 mg, 42%) as a white solid. ESI-MS [M+H]+: 260.1

### Synthesis of 3-(2-(aminomethyl)-6-cyclopropylimidazo[1,2-a]pyridin-8-yl)propanenitrile

A mixture of 3-(2-(chloromethyl)-6-cyclopropylimidazo[1,2-a]pyridin-8-yl)propanenitrile (70 mg, 0.27 mmol) in NH₃ (2N NH₃ in ⁱPrOH, 20 mL, 40 mmol) was stirred at 60° C. in sealed tube for 14 h. After cooling to room temperature, the reaction mixture was concentrated in vacuo to give the 3-(2-(aminomethyl)-6-cyclopropylimidazo[1,2-a]pyridin-8-yl)propanenitrile (80 mg, quant), which was used without further purification. ESI-MS [M+H]+: 241.1

### Synthesis of 3-(6-cyclopropyl-2-(hydroxymethyl)imidazo[1,2-a]pyridin-8-yl)propanenitrile

A mixture of 3-(2-(chloromethyl)-6-cyclopropylimidazo[1,2-a]pyridin-8-yl)propanenitrile (0.13 g, 0.50 mmol) and Na₂CO₃ (0.16 g, 1.5 mmol) in THF (10 mL) and water (10 mL) was stirred at 90° C. for 16 h. After cooling to room temperature, the reaction mixture was quenched with water (10 mL) and extracted with EtOAc/MeOH (10/1, 3×20 mL). The combined organic layers were washed with saturated brine solution (20 mL), dried over Na₂SO₄, and concentrated in vacuo to give 3-(6-cyclopropyl-2-(hydroxymethyl)imidazo[1,2-a]pyridin-8-yl)propanenitrile as a white solid, which was used into next step without further purification. (90 mg, 75%) ESI-MS [M+H]+: 241.2

### Synthesis of (R)-1-(2-(1-aminoethyl)-6-(1,1-difluoroethyl)imidazo[1,2-a]pyridin-8-yl)-3-methylimidazolidine-2,4-dione

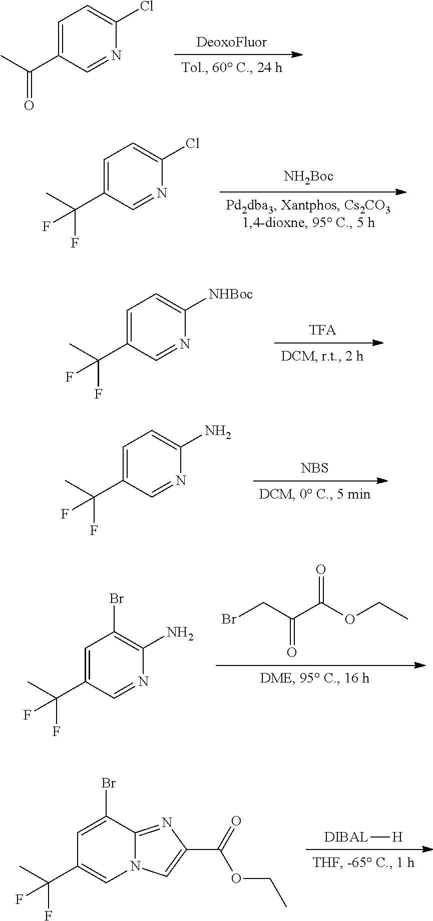

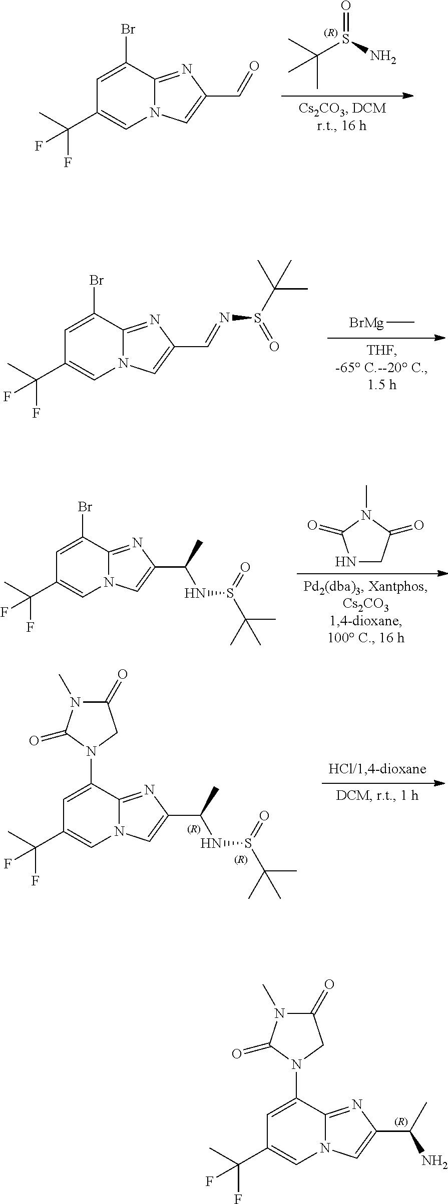

### Synthesis of 2-chloro-5-(1,1-difluoroethyl)pyridine

To a solution of 1-(6-chloropyridin-3-yl)ethan-1-one (6.0 g, 39 mmol) in toluene (80 mL) was added DeoxoFluor® (21 mL, 0.11 mol) at room temperature and the mixture was stirred at 60° C. for 24 h. The reaction was quenched with NaHCO₃(Sat·aq·, 50 mL) and extracted With EtOAc (50 mL×3). The combined organic layers were washed with brine (50 mL), dried over Na₂SO₄ and concentrated. The residue was purified by column chromatography (eluent: EtOAc/PE=0-20%) to afford 2-chloro-5-(1,1-difluoroethyl)pyridine (4.3 g, yield 62%) as colorless oil. ESI-MS [M+H]+: 178.1

### Synthesis of tert-butyl (5-(1,1-difluoroethyl)pyridin-2-yl)carbamate

To a mixture of 2-chloro-5-(1,1-difluoroethyl)pyridine (4.3 g, 24 mmol) and tert-butyl carbamate (5.7 g, 48 mmol), Xantphos (2.8 g, 4.8 mmol) and Cs₂CO₃ (20 g, 61 mmol) in 1,4-dioxane (100 mL) was added Pd₂(dba)₃ (2.2 g, 2.4 mmol) and the mixture was stirred at 95° C. for 5 h under N₂. The reaction mixture was filtered through Celite® and the filter cake was washed with DCM/MeOH (10/1, 100 mL). The filtrate was concentrated in vacuo to give the crude, which was purified by column chromatography (eluent: EtOAc/PE=0-20%) to afford tert-butyl (5-(1,1-difluoroethyl)pyridin-2-yl)carbamate (6 g, yield 97%) as a pale yellow solid. ESI-MS [M+H]+: 259.1

### Synthesis of 5-(1,1-d fluoroethyl)pyridin-2-amine

To a solution of tert-butyl (5-(1,1-difluoroethyl)pyridin-2-yl)carbamate (6 g, 23 mmol) in DCM (30 mL) was added TFA (5 mL) and the mixture was stirred at room temperature for 2 h. The reaction was concentrated in vacuo and then neutralized with NH₃ (10 mL, 7M in MeOH), and then concentrated in vacuo. The residue was purified by column chromatography (eluent: EtOAc/PE=0-50%) to afford 5-(1,1-difluoroethyl)pyridin-2-amine (2.6 g, yield 72%) as a pale yellow solid. ESI-MS [M+H]+: 159.1

### Synthesis of 3-bromo-5-(1,1-difluoroethyl)pyridin-2-amine

To a mixture of 5-(1,1-difluoroethyl)pyridin-2-amine (2.6 g, 16.5 mmol) in DCM (35 mL) was added NBS (3.2 g, 18 mmol) at 0° C. The mixture was stirred at 0° C. for 5 min and quenched with NaHCO₃(Sat·aq·, 50 mL). The mixture was extracted with DCM (50 mL×3). The combined organic layers were washed with brine (50 mL), dried over Na₂SO₄ and concentrated in vacuo. The residue was purified by column chromatography (eluent: EtOAc/PE=0-40%) to afford 3-bromo-5-(1,1-difluoroethyl)pyridin-2-amine (2.9 g, yield 75%) as pale yellow solid. ESI-MS [M+H]+: 237.1

### Synthesis of ethyl 8-bromo-6-(1,1-difluoroethyl)imidazo[1,2-a]pyridine-2-carboxylate

To a mixture of 3-bromo-5-(1,1-difluoroethyl)pyridin-2-amine (2.9 g, 12 mmol) in DME (50 mL) was added ethyl 3-bromo-2-oxopropanoate (2.6 mL, 18 mmol) and the mixture was stirred at 95° C. for 16 h. The mixture was quenched with NaHCO₃ (sat·aq·, 50 mL) and extracted with DCM (50 mL×3). The combined organic layers were washed with brine (50 mL), dried over Na₂SO₄ and concentrated in vacuo. The residue was purified by column chromatography (eluent: EtOAc/PE=0-50%) to afford ethyl 8-bromo-6-(1,1-difluoroethyl)imidazo[1,2-a]pyridine-2-carboxylate (3 g, yield 75%) as green sticky oil. ESI-MS [M+H]+: 333.1

### Synthesis of 8-bromo-6-(1,1-difluoroethyl)imidazo[1,2-a]pyridine-2-carbaldehyde carboxylate

To a mixture of ethyl 8-bromo-6-(1,1-difluoroethyl)imidazo[1,2-a]pyridine-2-carboxylate (1.5 g, 4.5 mmol) in THF (40 mL) was added DIBAL-H (14 mL, 14 mmol, 1 M in hexanes) at −65° C. and the mixture was stirred at −65° C. for 1 h. The reaction was quenched at −65° C. with MeOH (5 mL) and 15% NaOH (aq·, 20 mL). The mixture was allowed to warm to room temperature and extracted with EtOAc (50 mL×3). The combined organic layers were washed with brine (50 mL), dried over Na₂SO₄ and concentrated in vacuo. The residue was purified by column chromatography (eluent: EtOAc/PE=0-35%) to afford 8-bromo-6-(1,1-difluoroethyl)imidazo[1,2-a]pyridine-2-carbaldehyde carboxylate (560 mg, yield 43%) as pale-yellow solid. ESI-MS [M+H]+: 289.1

### Synthesis of (R,E)-N-((8-bromo-6-(1,1-difluoroethyl)imidazo[1,2-a]pyridin-2-yl)methylene)-2-methylpropane-2-sulfinamide

To a mixture of 8-bromo-6-(1,1-difluoroethyl)imidazo[1,2-a]pyridine-2-carbaldehyde carboxylate (560 mg, 1.94 mmol) and (R)-2-methylpropane-2-sulfinamide (280 mg, 2.33 mmol) in DCM (10 mL) was added Cs₂CO₃ (1.3 g, 3.88 mmol) and the mixture was stirred at room temperature for 16 h. The reaction was filtered, and the filtrate was washed with DCM (50 mL) then concentrated in vacuo. The residue was purified by column chromatography (eluent: EtOAc/PE=0-40%) to afford (R,E)-N-((8-bromo-6-(1,1-difluoroethyl)imidazo[1,2-a]pyridin-2-yl)methylene)-2-methylpropane-2-sulfinamide (750 mg, yield 99%) as white solid. ESI-MS [M+H]+: 392.1

### Synthesis of (R)—N—((R)-1-(8-bromo-6-(1,1-difluoroethyl)imidazo[1,2-a]pyridin-2-yl)ethyl)-2-methylpropane-2-sulfinamide

To a solution of (R,E)-N-((8-bromo-6-(1,1-difluoroethyl)imidazo[1,2-a]pyridin-2-yl)methylene)-2-methylpropane-2-sulfinamide (750 mg, 1.92 mmol) in THF (20 mL) was added dropwise methylmagnesium bromide (2 mL, 6 mmol, 3M in ether) at −65° C. The mixture was warmed to −20° C. and stirred for 1.5 h. The reaction was quenched with NH₄Cl (Sat·aq·, 50 mL) and extracted with EtOAc (50 mL×3). The combined organic layers were washed with brine (50 mL), dried over Na₂SO₄ and concentrated in vacuo. The residue was purified by column chromatography (eluent: EtOAc/PE=0-50%) to afford (R)—N—((R)-1-(8-bromo-6-(1,1-difluoroethyl)imidazo[1,2-a]pyridin-2-yl)ethyl)-2-methylpropane-2-sulfinamide (550 mg, yield 64%) as white solid. ESI-MS [M+H]+: 408.1

### Synthesis of (R)—N—((R)-1-(6-(1,1-difluoroethyl)-8-(3-methyl-2,4-dioxoimidazolidin-1-yl)imidazo[1,2-a]pyridin-2-yl)ethyl)-2-methylpropane-2-sulfinamide

To a mixture of (R)—N—((R)-1-(8-bromo-6-(1,1-difluoroethyl)imidazo[1,2-a]pyridin-2-yl)ethyl)-2-methylpropane-2-sulfinamide (550 mg, 1.35 mmol) and 3-methylimidazolidine-2,4-dione (610 mg, 5.35 mmol) in 1,4-dioxane (25 mL) was added Pd₂(dba)₃ (180 mg, 0.21 mmol), xantPhos (0.23 g, 0.40 mmol) and Cs₂CO₃ (1.1 g, 3.4 mmol). The mixture was stirred at 100° C. for 16 h under N₂. The reaction mixture was filtered through Celite® and the filter cake was washed with DCM/MeOH (10/1, 100 mL). The filtrate was concentrated in vacuo to give the crude, which was purified by column chromatography (eluent: EtOAc/PE=0-50%) to afford (R)—N—((R)-1-(6-(1,1-difluoroethyl)-8-(3-methyl-2,4-dioxoimidazolidin-1-yl)imidazo[1,2-a]pyridin-2-yl)ethyl)-2-methylpropane-2-sulfinamide (500 mg, yield 84%) as pale yellow solid. ESI-MS [M+H]+: 442.2

### Synthesis of (R)-1-(2-(1-aminoethyl)-6-(1,1-difluoroethyl)imidazo[1,2-a]pyridin-8-yl)-3-methylimidazolidine-2,4-dione

To a mixture of (R)—N—((R)-1-(6-(1,1-difluoroethyl)-8-(3-methyl-2,4-dioxoimidazolidin-1-yl)imidazo[1,2-a]pyridin-2-yl)ethyl)-2-methylpropane-2-sulfinamide (500 mg, 1.13 mmol) in DCM (4 mL) was added HCl (4 mL, 16 mmol, 4M in 1,4-dioxane) at 0° C. The reaction was stirring at room temperature for 1 h. The reaction was concentrated in vacuo. The residue was neutralized with NH₃ (10 mL, 7 M in MeOH). The reaction was stirred for 5 min and then concentrated in vacuo. The residue was purified by column chromatography (eluent: MeOH/DCM=0-9%) to afford (R)-1-(2-(1-aminoethyl)-6-(1,1-difluoroethyl)imidazo[1,2-a]pyridin-8-yl)-3-methylimidazolidine-2,4-dione (340 mg, yield 89%) as white solid. ESI-MS [M+H]+: 338.2

### Synthesis of 1-(2-((R)-1-aminoethyl)-6-(1-fluoroethyl)imidazo[1,2-a]pyridin-8-yl)-3-methylimidazolidine-2,4-dione

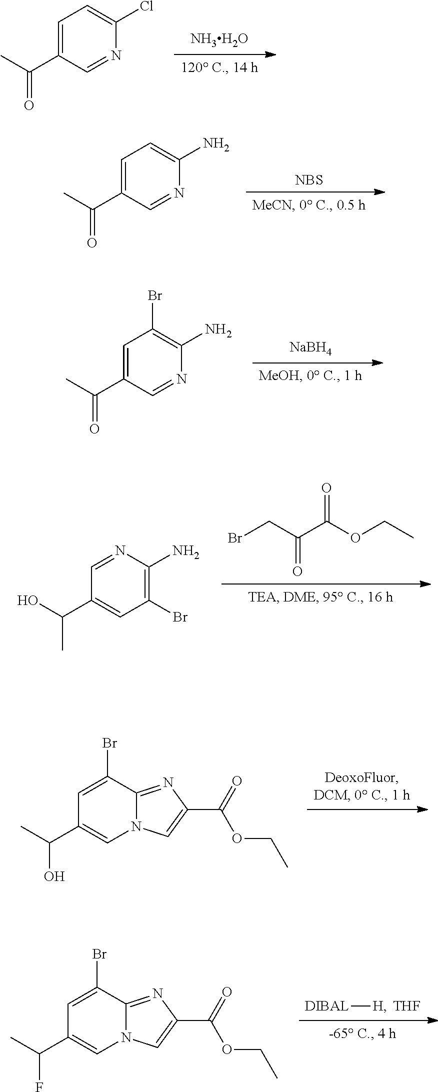

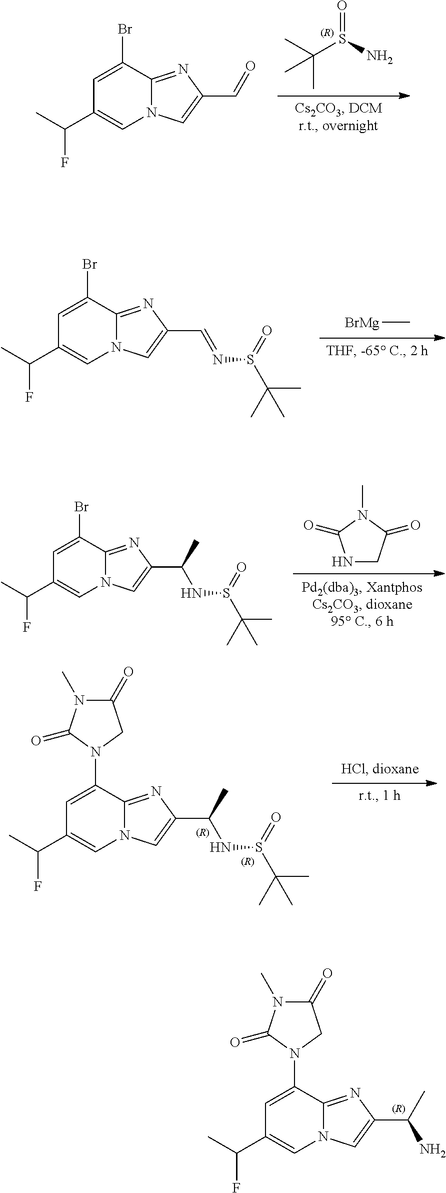

### Synthesis of 1-(6-aminopyridin-3-yl)ethan-1-one

A mixture of 1-(6-chloropyridin-3-yl)ethan-1-one (4.96 g, 32 mmol) in NH₃/H₂O (60 mL) was stirred in a sealed tube at 120° C. for 14 h. After cooling to room temperature, the reaction mixture was concentrated in vacuo to give the crude, which was purified by column chromatography (eluent: MeOH/DCM=0˜5%) to give 1-(6-aminopyridin-3-yl)ethan-1-one (3.4 g, 78%) as a yellow solid. ESI-MS [M+H]+: 137.1.

### Synthesis of 1-(6-amino-5-bromopyridin-3-yl)ethan-1-one

To a stirred solution of 1-(6-aminopyridin-3-yl)ethan-1-one (3.4 g, 25 mmol) in MeCN (120 mL) was added NBS (4.9 g in 30 mL MeCN, 27.5 mmol) at 0° C. slowly. Then the reaction was stirred at 0° C. for 0.5 h. The reaction mixture was quenched with Na₂SO₃ (sat·aq·, 100 mL) and extracted with EtOAc (50 mL×3). The combined organic layers were washed with brine (50 mL), dried over Na₂SO₄ and concentrated in vacuo to give the crude, which was purified by column chromatography (eluent: EtOAc/PE=0˜20%) to give the 1-(6-amino-5-bromopyridin-3-yl)ethan-1-one (4.3 g, 80%) as yellow solid. ESI-MS [M+H]⁺: 215.0.

### Synthesis of 1-(6-amino-5-bromopyridin-3-yl)ethan-1-ol

To a solution of 1-(6-amino-5-bromopyridin-3-yl)ethan-1-one (4.3 g, 20 mmol) in MeOH (100 mL) was added NaBH₄ (2.28 g, 60 mmol) portionwise at 0° C. The resulting mixture was stirred at 0° C. for 1 h. The reaction was quenched with NH₄Cl (sat·aq·, 100 mL) and extracted with EtOAc (60 mL×3). The combined organic layers were washed with brine (60 mL), dried over Na₂SO₄ and concentrated in vacuo to give the crude, which was purified by column chromatography (eluent: MeOH/DCM=0˜5%) to give 1-(6-amino-5-bromopyridin-3-yl)ethan-1-ol (2.9 g, 67%) as yellow solid. ESI-MS [M+H]⁺: 217.0.

### Synthesis of ethyl 8-bromo-6-(1-hydroxyethyl)imidazo[1,2-a]pyridine-2-carboxylate

To a solution of 1-(6-amino-5-bromopyridin-3-yl)ethan-1-ol (2.5 g, 11.5 mmol) in DME (100 mL) was added ethyl 3-bromo-2-oxopropanoate (6.7 g, 34.5 mmol) and TEA (3.5 g, 34.5 mmol). The reaction was stirred at 95° C. for 16 h. After cooling to room temperature, the reaction mixture was quenched with NaHCO₃ (sat·aq·, 100 mL) and extracted with EtOAc (50 mL×3). The combined organic layers were washed with brine (50 mL), dried over Na₂SO₄ and concentrated in vacuo to give the crude, which was purified by column chromatography (eluent: EtOAc/PE=0˜30%) to give the ethyl 8-bromo-6-(1-hydroxyethyl)imidazo[1,2-a]pyridine-2-carboxylate (1.3 g, 36%) as yellow oil. ESI-MS [M+H]+: 313.0.

### Synthesis of ethyl 8-bromo-6-(1-fluoroethyl)imidazo[1,2-a]pyridine-2-carboxylate

To a solution of ethyl 8-bromo-6-(1-hydroxyethyl)imidazo[1,2-a]pyridine-2-carboxylate (1.3 g, 4.2 mmol) in DCM (80 mL) was added DeoxoFluor® (2.8 g, 12.6 mol) at 0° C. The resulting mixture was stirred at 0° C. for 1 h. The reaction was quenched with NaHCO₃ (sat·aq·, 80 mL) and extracted with EtOAc (50 mL×3). The combined organic layers were washed with brine (50 mL), dried over Na₂SO₄ and concentrated in vacuo to give the crude, which was purified by column chromatography (eluent: EtOAc/PE=0˜20%) to give ethyl 8-bromo-6-(1-fluoroethyl)imidazo[1,2-a]pyridine-2-carboxylate (800 mg, 61%) as yellow oil. ESI-MS [M+H]+: 314.9.

### Synthesis of 8-bromo-6-(1-fluoroethyl)imidazo[1,2-a]pyridine-2-carbaldehyde

To a stirred solution of ethyl 8-bromo-6-(1-fluoroethyl)imidazo[1,2-a]pyridine-2-carboxylate (800 mg, 2.5 mmol) in THF (50 mL) was slowly added DIBAL-H (7.5 mL, 7.5 mmol, 1 mol/L) at −70° C. Then the reaction was stirred at −65° C. for 4h. EtOAc (50 mL) and Na₂SO₄·10H₂O was added in slowly at −65° C. and the reaction mixture was warmed to room temperature and stirred for 30 minutes. Water (10 mL) was added slowly at room temperature and the reaction mixture was stirred at room temperature stirred for another 10 minutes. The reaction mixture was filtered through Celite® and the filter cake was washed with DCM/MeOH (10/1, 50 mL). The filtrate was concentrated in vacuo to give the crude, which was purified by silica gel chromatography (eluent: EtOAc/PE=0˜20%) to give the 8-bromo-6-(1-fluoroethyl)imidazo[1,2-a]pyridine-2-carbaldehyde (440 mg, 65%) as yellow solid. ESI-MS [M+H]+: 271.0.

### Synthesis of ((R)—N-((E)-(8-bromo-6-(1-fluoroethyl)imidazo[1,2-a]pyridin-2-yl)methylene)-2-methylpropane-2-sulfinamide

To a reaction mixture of 8-bromo-6-(1-fluoroethyl)imidazo[1,2-a]pyridine-2-carbaldehyde (432 mg, 1.6 mmol) and (R)-2-methylpropane-2-sulfinamide (290 mg, 2.4 mmol) in DCM (15 mL) was added Cs₂CO₃ (1.04 g, 3.2 mol) and the mixture was stirred at room temperature for 16 h. The mixture was quenched water (50 mL) and extracted with DCM (50 mL×3). The combined organic layers were washed with brine (50 mL), dried over Na₂SO₄ and concentrated in vacuo. The residue was purified by column chromatography (eluent: EtOAc/PE=0˜50%) to give ((R)—N-((E)-(8-bromo-6-(1-fluoroethyl)imidazo[1,2-a]pyridin-2-yl)methylene)-2-methylpropane-2-sulfinamide (320 mg, 54%) as yellow solid. ESI-MS [M+H]+: 374.0.

### Synthesis of (R)—N-((1R)-1-(8-bromo-6-(1-fluoroethyl)imidazo[1,2-a]pyridin-2-yl)ethyl)-2-methylpropane-2-sulfinamide

To a stirred solution of ((R)—N-((E)-(8-bromo-6-(1-fluoroethyl)imidazo[1,2-a]pyridin-2-yl)methylene)-2-methylpropane-2-sulfinamide (320 mg, 0.86 mmol) in THF (10 mL) was slowly added methylmagnesium bromide (1.2 mL, 3.6 mmol, 3 mol/L) at −70° C. Then the reaction was stirred at −65° C. for 2 h. The reaction was quenched with NH₄Cl (sat·aq·, 30 mL) and then extracted with EtOAc (30 mL×3). The combined organic layers were washed with brine (30 mL), dried over Na₂SO₄, filtered and concentrated in vacuo to give the crude, which was purified by column chromatography (eluent: MeOH/DCM=0˜5%) to give (R)—N-((1R)-1-(8-bromo-6-(1-fluoroethyl)imidazo[1,2-a]pyridin-2-yl)ethyl)-2-methylpropane-2-sulfinamide (200 mg, 60%) as yellow solid. ESI-MS [M+H]+: 390.1.

### Synthesis of (R)—N-((1R)-1-(6-(1-fluoroethyl)-8-(3-methyl-2,4-dioxoimidazolidin-1-yl)imidazo[1,2-a]pyridin-2-yl)ethyl)-2-methylpropane-2-sulfinamide

To a stirred mixture of (R)—N-((1R)-1-(8-bromo-6-(1-fluoroethyl)imidazo[1,2-a]pyridin-2-yl)ethyl)-2-methylpropane-2-sulfinamide (200 mg, 0.5 mmol), 3-methylimidazolidine-2,4-dione (171 mg, 1.5 mmol), xantPhos (58 mg, 0.1 mmol) and Cs₂CO₃ (489 mg, 1.5 mmol) in 1,4-dioxane (15 mL) was added Pd₂(dba)₃ (46 mg, 0.05 mmol). The reaction was stirred at 95° C. for 6 h under N₂. The reaction mixture was cooled to room temperature then filtered through Celite® and the filter cake was washed with DCM/MeOH (10/1, 50 mL). The filtrate was concentrated in vacuo to give the crude, which was purified by column chromatography (eluent: MeOH/DCM=0˜3%) to give the (R)—N-((1R)-1-(6-(1-fluoroethyl)-8-(3-methyl-2,4-dioxoimidazolidin-1-yl)imidazo[1,2-a]pyridin-2-yl)ethyl)-2-methylpropane-2-sulfinamide (195 mg, 92%) as yellow solid. ESI-MS [M+H]+: 424.1.

### Synthesis of 1-(2-((R)-1-aminoethyl)-6-(1-fluoroethyl)imidazo[1,2-a]pyridin-8-yl)-3-methylimidazolidine-2,4-dione

To a solution of (R)—N-((1R)-1-(6-(1-fluoroethyl)-8-(3-methyl-2,4-dioxoimidazolidin-1-yl)imidazo[1,2-a]pyridin-2-yl)ethyl)-2-methylpropane-2-sulfinamide (195 mg, 0.46 mol) in 1,4-dioxane (8 mL) was added HCl (2 mL, 8 mmol, 4M solution in 1,4-dioxane). The resulting mixture was stirred at room temperature for 1 h. The reaction was concentrated in vacuo and neutralized with NH₃ (5 mL, 7 M in MeOH). The reaction was stirred for 5 min and concentrated in vacuo to give the crude, which was purified by column chromatography (eluent: MeOH/DCM=0˜10%) to afford 1-(2-((R)-1-aminoethyl)-6-(1-fluoroethyl)imidazo[1,2-a]pyridin-8-yl)-3-methylimidazolidine-2,4-dione (100 mg, 68%) as yellow solid. ESI-MS [M+H]+: 320.1.

### Synthesis of 1-(2-(aminomethyl)-6-(2,2-difluorocyclopropyl)imidazo[1,2-a]pyridin-8-yl)-3-methylimidazolidine-2,4-dione

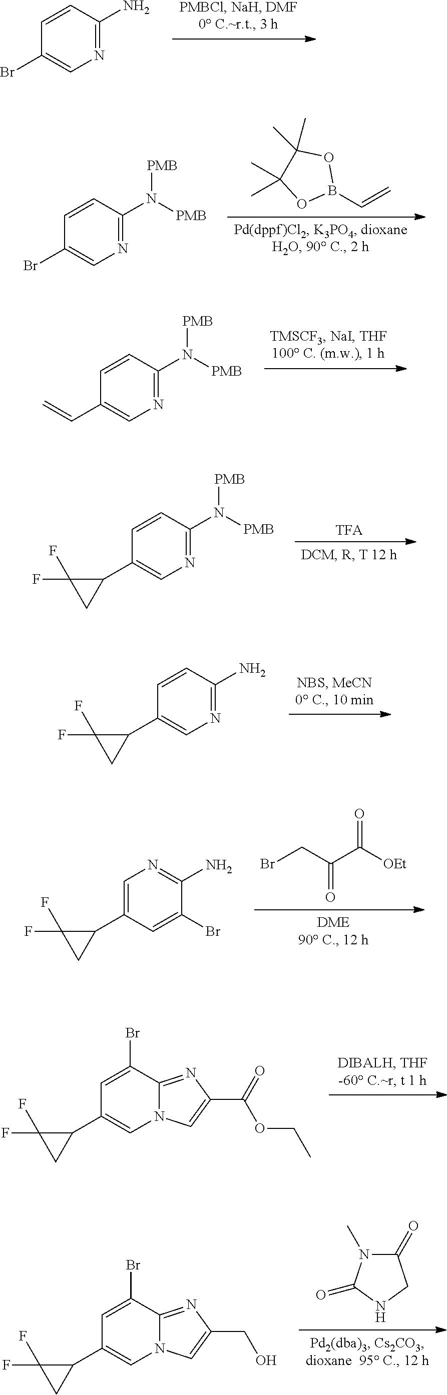

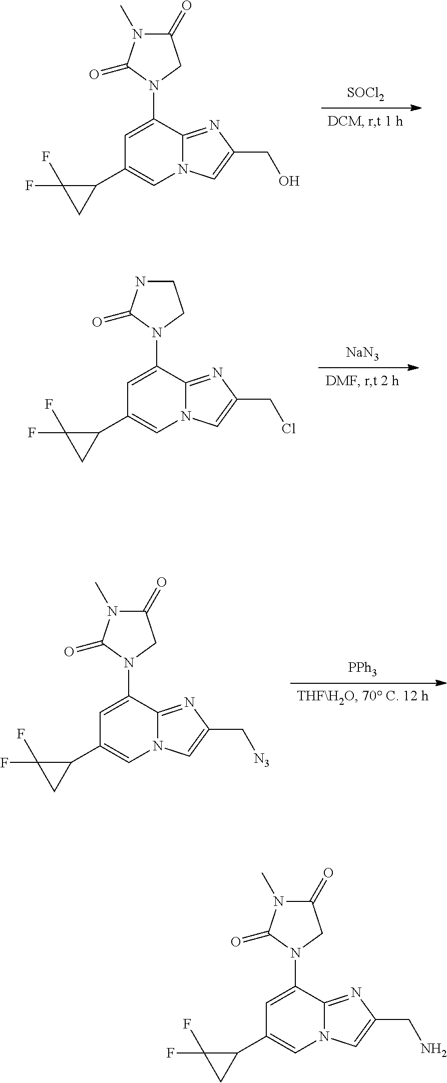

### Synthesis of 5-bromo-N,N-bis(4-methoxybenzyl)pyridin-2-amine

To a mixture of 5-bromopyridin-2-amine (5 g, 28.9 mmol) in DMF (80 ml) was added NaH (4.62 g, 60% in mineral oil, 115.6 mmol) at 0° C. and the mixture was stirred at 0° C. for 1 h. PMBCl (18 g, 115.6 mmol) was added and the resulting mixture was stirred at room temperature for 2 h. The mixture was quenched with NH₄Cl (sat·aq·, 10 mL) and extracted with EtOAc (100 mL×3). The combined organic layers were washed with brine (100 mL), dried over Na₂SO₄ and concentrated in vacuo. The residue was purified by column chromatography (eluent: EtOAc/PE=0˜20%) to give 5-bromo-N,N-bis(4-methoxybenzyl)pyridin-2-amine (10 g, 84%) as a white solid. ESI-MS [M+H]+: 413.2.

### Synthesis of N,N-bis(4-methoxybenzyl)-5-vinylpyridin-2-amine

To a mixture of 5-bromo-N,N-bis(4-methoxybenzyl)pyridin-2-amine (2 g, 4.8 mmol), 4,4,5,5-tetramethyl-2-vinyl-1,3,2-dioxaborolane (1.1 g, 7.3 mmol) and K₃PO₄ (2.1 g, 9.6 mmol) in 1,4-dioxane/H₂O (40/10 mL) was added Pd(dppf)Cl₂ (350.8 mg, 0.48 mmol). The resulting reaction mixture was stirred at 90° C. for 2 h under N₂. After cooling to room temperature, water (100 mL) was added and the mixture was extracted with EtOAc (40 mL×3). The combined organic layers were washed with brine (60 mL), dried over anhydrous Na₂SO₄, and concentrated in vacuo to give the crude, which was purified by column chromatography (eluent: EtOAc/PE=0˜20%) to give N,N-bis(4-methoxybenzyl)-5-vinylpyridin-2-amine (1.1 g, 64%) as a yellow solid. ESI-MS [M+H]⁺: 361.2.

### Synthesis of 5-(2,2-difluorocyclopropyl)-N,N-bis(4-methoxybenzyl)pyridin-2-amine

A mixture of N,N-bis(4-methoxybenzyl)-5-vinylpyridin-2-amine (800 mg, 2.2 mmol), TMSCF3 (1.25 g, 8.8 mmol), NaI (1.32 g, 8.8 mmol) in THF (3 mL) in a sealed tube was irradiated in a microwave reactor at 100° C. for 4 h. The mixture was quenched with NaHCO₃ (sat·aq·, 50 mL) and extracted with DCM (50 mL×3). The combined organic layers were washed with brine (50 mL), dried over Na₂SO₄ and concentrated in vacuo. The residue was purified by column chromatography (eluent: EtOAc/PE=0-30%) to give 5-(2,2-difluorocyclopropyl)-N,N-bis(4-methoxybenzyl)pyridin-2-amine (480 mg, 53%) as a yellow solid. ESI-MS [M+H]⁺: 411.2.

### Synthesis of 5-(2,2-difluorocyclopropyl)pyridin-2-amine

A solution of 5-(2,2-difluorocyclopropyl)-N,N-bis(4-methoxybenzyl)pyridin-2-amine (6 g, 14.6 mmol) in TFA (20 mL) and DCM (20 mL) was stirred at room temperature for 12 h. The reaction was concentrated in vacuo. The residue was diluted with NaHCO₃ (sat·aq·, 100 mL) and extracted with DCM (100 mL×3). The combined organic layers were washed with brine (100 mL), dried over Na₂SO₄ and concentrated in vacuo. The residue was purified by column chromatography (eluent: MeOH/DCM=0-10%) to give 5-(2,2-difluorocyclopropyl)309yridine-2-amine (2 g, 80%) as a white solid. ESI-MS [M+H]⁺: 171.2

### Synthesis of 3-bromo-5-(2,2-difluorocyclopropyl)309yridine-2-amine

To a mixture of 5-(2,2-difluorocyclopropyl)309yridine-2-amine (2 g, 11.8 mmol) in MeCN (40 mL) was added NBS (2.3 g, 13.0 mmol) at 0° C. and the reaction mixture was stirred at 0° C. for 10 min. The mixture was quenched with Na₂SO₃ (sat·aq·, 100 mL) and extracted with DCM (100 mL×3). The combined organic layers were washed with brine (100 mL), dried over Na₂SO₄ and concentrated in vacuo. The residue was purified by column chromatography (eluent: MeOH/DCM=0-10%) to give 3-bromo-5-(2,2-difluorocyclopropyl)309yridine-2-amine (1.4 g, 48%) as a yellow solid. ESI-MS [M+H]⁺: 249.2.

### Synthesis of ethyl 8-bromo-6-(2,2-difluorocyclopropyl)imidazo[1,2-a]pyridine-2-carboxylate

A mixture of 3-bromo-5-(2,2-difluorocyclopropyl)pyridin-2-amine (800 mg, 3.2 mmol) and ethyl 3-bromo-2-oxopropanoate (1.86 g, 9.6 mmol) in DME (16 mL) was stirred at 90° C. for 12 h. The mixture was quenched with NaHCO₃ (sat·aq·, 100 mL) and extracted with EtOAc (100 mL×3). The combined organic layers were washed with brine (100 mL), dried over Na₂SO₄ and concentrated in vacuo. The residue was purified by column chromatography (eluent: EtOAc/PE=0-50%) to give ethyl 8-bromo-6-(2,2-difluorocyclopropyl)imidazo[1,2-a]pyridine-2-carboxylate (500 mg, 45%) as a yellow solid. ESI-MS [M+H]⁺: 345.2

### Synthesis of (8-bromo-6-(2,2-difluorocyclopropyl)imidazo[1,2-a]pyridin-2-yl)methanol

To a solution of ethyl 8-bromo-6-(2,2-difluorocyclopropyl)imidazo[1,2-a]pyridine-2-carboxylate (100 mg, 0.29 mmol) in THF (3 mL) was added DIBALH (1.0 M solution in hexane, 0.87 mL, 0.87 mmol) at −60° C. The resulting reaction was warmed and stirred at room temperature for 1 h. Na₂SO₄·10H₂O was added and the resulting mixture was stirred at room temperature for 0.5h. The mixture was filtered and the filter cake was washed with DCM/MeOH (10/1, 30 mL). The filtrate was concentrated in vacuo to give the crude, which was purified by preparative TLC (eluent: DCM/MeOH=30/1) to give (8-bromo-6-(2,2-difluorocyclopropyl)imidazo[1,2-a]pyridin-2-yl)methanol (50 mg, 57%) as a white solid. ESI-MS [M+H]+: 303.1.

### Synthesis of 1-(6-(2,2-difluorocyclopropyl)-2-(hydroxymethyl)imidazo[1,2-a]pyridin-8-yl)-3-methylimidazolidine-2,4-dione

To a mixture of (8-bromo-6-(2,2-difluorocyclopropyl)imidazo[1,2-a]pyridin-2-yl)methanol (300 mg, 0.99 mmol), 3-methylimidazolidine-2,4-dione (340 mg, 2.97 mmol), Cs₂CO₃ (968 mg, 2.97 mmol), and xantPhos (115 mg, 0.2 mmol) in 1,4-dioxane (10 mL) was added Pd₂(dba)₃ (137 mg, 0.15 mmol) and the reaction mixture was stirred at 95° C. for 12 h under N₂. The reaction mixture was filtered through Celite® and the filter cake was washed with DCM/MeOH (10/1, 100 mL). The filtrate was concentrated in vacuo to give the crude, which was purified by preparative TLC (eluent: DCM/MeOH=20/1) to give 1-(6-(2,2-difluorocyclopropyl)-2-(hydroxymethyl)imidazo[1,2-a]pyridin-8-yl)-3-methylimidazolidine-2,4-dione (180 mg, 54%) as a yellow solid. ESI-MS [M+H]+: 337.2.

### Synthesis of 1-(2-(chloromethyl)-6-(2,2-difluorocyclopropyl)imidazo[1,2-a]pyridin-8-yl)-312-imidazolidin-2-one

To a solution of 1-(6-(2,2-difluorocyclopropyl)-2-(hydroxymethyl)imidazo[1,2-a]pyridin-8-yl)-3-methylimidazolidine-2,4-dione (150 mg, 0.45 mmol) in DCM (5 mL) was added (108 mg, 0.9 mmol). The reaction mixture was stirred at room temperature for 1 h under N₂ and concentrated in vacuo to give 1-(2-(chloromethyl)-6-(2,2-difluorocyclopropyl)imidazo[1,2-a]pyridin-8-yl)-312-imidazolidin-2-one (160 mg, crude) as a yellow solid, which was used in the next step without further purification. ESI-MS [M+H]+: 326.2.

### Synthesis of 1-(2-(chloromethyl)-6-(2,2-difluorocyclopropyl)imidazo[1,2-a]pyridin-8-yl)-312-imidazolidin-2-one

A mixture of 1-(2-(chloromethyl)-6-(2,2-difluorocyclopropyl)imidazo[1,2-a]pyridin-8-yl)-312-imidazolidin-2-one (160 mg, crude) and NaN₃ (64 mg, 0.98 mmol) in DMF (3 mL) was stirred at room temperature for 2 h. The mixture was quenched with water (30 mL) and extracted with EtOAc (30 mL×3). The combined organic layers were washed with brine (20 mL), dried over Na₂SO₄ and concentrated in vacuo to give methyl 1-(2-(azidomethyl)-6-(2,2-difluorocyclopropyl)imidazo[1,2-a]pyridin-8-yl)-3-methylimidazolidine-2,4-dione (165 mg, crude) as a yellow solid, which was used in the next step without further purification. ESI-MS [M+H]+: 362.1.

### Synthesis of 1-(2-(aminomethyl)-6-(2,2-difluorocyclopropyl)imidazo[1,2-a]pyridin-8-yl)-3-methylimidazolidine-2,4-dione

To a mixture of methyl 1-(2-(azidomethyl)-6-(2,2-difluorocyclopropyl)imidazo[1,2-a]pyridin-8-yl)-3-methylimidazolidine-2,4-dione (160 mg, crude) in THF/H₂O (10 ml/1 mL) was added PPh₃ (254 mg, 0.97 mmol) and the reaction mixture was stirred at 70° C. for 2 h under N₂. Water (20 mL) was added and the mixture was extracted with EtOAc (20 mL×5). The combined organic layers were washed with brine (20 mL), dried over anhydrous Na₂SO₄, and concentrated in vacuo to give the crude, which was purified by preparative TLC (eluent: DCM/MeOH=20/1) to give 1-(2-(aminomethyl)-6-(2,2-difluorocyclopropyl)imidazo[1,2-a]pyridin-8-yl)-3-methylimidazolidine-2,4-dione (90 mg, 60% over 3 steps) as a yellow solid. ESI-MS [M+H]⁺: 336.2.

### Synthesis of N-(2-(((6-chloropyrimidin-4-yl)amino)methyl)-6-cyclopropylimidazo[1,2-a]pyridin-8-yl)acetamide

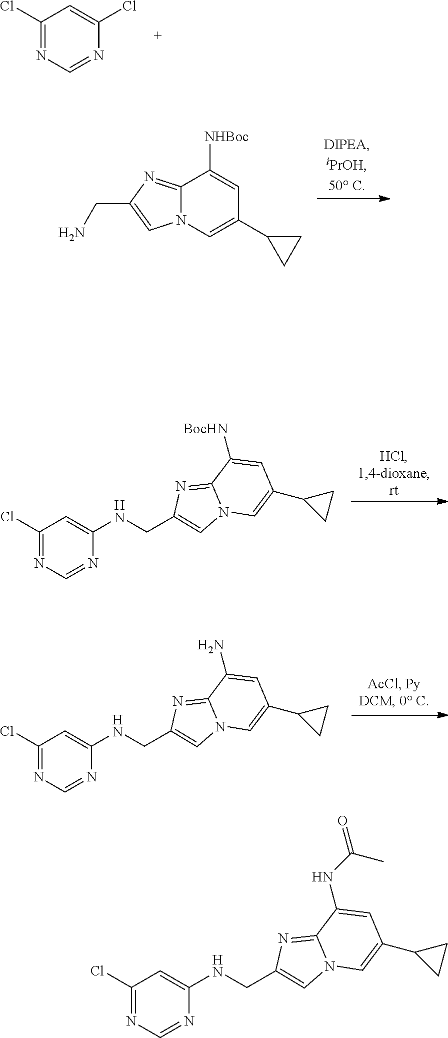

### Synthesis of tert-butyl (2-(((6-chloropyrimidin-4-yl)amino)methyl)-6-cyclopropylimidazo[1,2-a]pyridin-8-yl)carbamate

DIPEA (0.19 mL, 1.91 mmol) was added to a stirred solution of 4,6-dichloropyrimidine (95 mg, 0.64 mmol) and tert-butyl (2-(aminomethyl)-6-cyclopropylimidazo[1,2-a]pyridin-8-yl)carbamate (193 mg, 0.64 mmol) in isopropanol (5 mL), the reaction was then heated at 50° C. for 18 h. The reaction was cooled to room temperature and partitioned between water (30 mL) and EtOAc (30 mL), the layers were separated and the aqueous layer was further extracted with EtOAc (2×20 mL). The organics were combined, dried over MgSO₄ and concentrated in vacuo to give the title compound (175 mg, 66%). ESI-MS (M+H)+: 415.3, ¹H NMR (400 MHz, CDCl₃) δ 8.40 (s, 1H), 7.66 (s, 1H), 7.62 (s, 1H), 7.52 (d, J=5.1 Hz, 1H), 7.39 (s, 1H), 6.45 (d, J=0.8 Hz, 1H), 4.62 (s, 2H), 1.90-1.86 (m, 1H), 1.55 (s, 9H), 1.21 (d, J=7.5 Hz, 1H), 0.96-0.90 (m, 2H), 0.73-0.68 (m, 2H).

### Synthesis of 2-(((6-chloropyrimidin-4-yl)amino)methyl)-6-cyclopropylimidazo[1,2-a]pyridin-8-amine

4M HCl in dioxane (0.16 mL, 0.63 mmol) was added to a stirred solution of tert-butyl (2-(((6-chloropyrimidin-4-yl)amino)methyl)-6-cyclopropylimidazo[1,2-a]pyridin-8-yl)carbamate (175 mg, 0.42 mmol) in 1,4-dioxane (5.0 mL) and stirred at room temperature for 18 h. The reaction was quenched with NaHCO₃ solution (sat·aq·, 30 mL) and extracted with EtOAc (3×20 mL). The organics were combined, dried (MgSO₄) and concentrated in vacuo. The residue was purified by silica gel chromatography, eluting with a gradient of 0-10% MeOH (7N NH₃ in MeOH) in DCM to give the title compound (61 mg, 46%).

ESI-MS (M+H)+: 315.1, ¹H NMR (400 MHz, CDCl₃) δ 8.39 (s, 1H), 7.36 (d, J=7.8 Hz, 2H), 6.43 (s, 1H), 6.11 (d, J=1.5 Hz, 1H), 6.01 (s, 1H), 4.62-4.62 (m, 2H), 4.39 (s, 2H), 1.84-1.76 (m, 1H), 0.92-0.87 (m, 2H), 0.65-0.60 (m, 2H).

### Synthesis of N-(2-(((6-chloropyrimidin-4-yl)amino)methyl)-6-cyclopropylimidazo[1,2-a]pyridin-8-yl)acetamide

Acetyl chloride (11.2 μL, 0.16 mmol) was added to a stirred solution of 2-(((6-chloropyrimidin-4-yl)amino)methyl)-6-cyclopropylimidazo[1,2-a]pyridin-8-amine (50 mg, 0.16 mmol) and pyridine (38 μL, 0.48 mmol) in DCM (0.5 mL) at 0° C. The reaction was stirred at 0° C. for 1 hour. The reaction was diluted with water (10 mL) and extracted with DCM (3×10 mL). The organics were combined, dried over MgSO₄ and concentrated in vacuo. The residue was purified by silica gel chromatography, eluting with a gradient of 0-10% MeOH (7N NH₃ in MeOH) in DCM to give the title compound (35 mg, 56%).

ESI-MS (M+H)+: 397.1, ¹H NMR (400 MHz, CDCl₃) δ 8.43 (s, 1H), 8.39 (s, 1H), 7.95 (s, 1H), 7.58 (s, 1H), 7.41 (s, 1H), 6.43 (s, 1H), 5.97-5.97 (m, 1H), 4.63-4.63 (m, 2H), 2.27-2.26 (m, 3H), 1.88 (s, 1H), 0.98-0.91 (m, 2H), 0.73-0.66 (m, 2H).

### Synthesis of 6-chloro-N-((6-cyclopropyl-3-fluoroimidazo[1,2-a]pyridin-2-yl)methyl)pyrimidin-4-amine

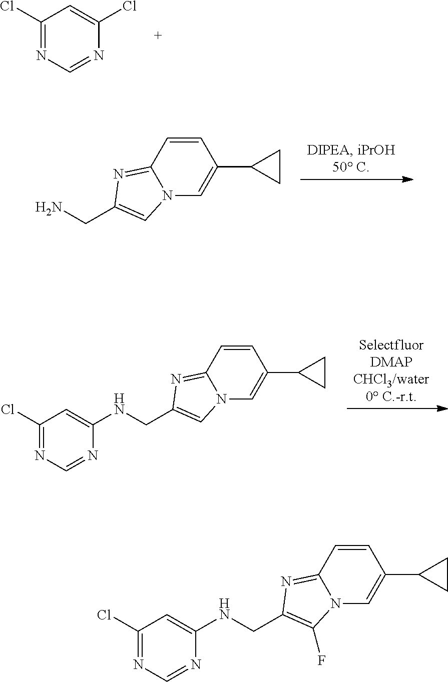 [SMILES: Clc1cc(Cl)ncn1 ; NCc1cn2cc(C3CC3)ccc2n1 ; "DIPEA, iPrOH50° C." ; Clc1cc(NCc2cn3cc(C4CC4)ccc3n2)ncn1 ; "SelectfluorDMAPCHCl3/water0° C.-r.t." ; Fc1c(CNc2cc(Cl)ncn2)nc2ccc(C3CC3)cn12]

### Synthesis of 6-chloro-N-((6-cyclopropylimidazo[1,2-a]pyridin-2-yl)methyl)pyrimidin-4-amine

A mixture of 4,6-dichloropyrimidine (1.8 g, 12 mmol), (6-cyclopropylimidazo[1,2-a]pyridin-2-yl)methanamine (2.5 g, 13 mmol) and DIPEA (6.3 mL, 36 mmol) in iPrOH (25 mL) was stirred at 50° C. for 18 h. Water was added and the precipitate was collected by filtration to give the title compound (3.2 g, 89%).

ESI-MS (M+H)+: 300, ¹H NMR (400 MHz, DMSO) δ 8.33-8.17 (m, 3H), 7.69 (s, 1H), 7.40 (d, J=9.4 Hz, 1H), 6.99 (dd, J=1.8, 9.2 Hz, 1H), 6.64 (d, J=0.7 Hz, 1H), 4.62-4.48 (m, 2H), 1.97-1.89 (m, 1H), 0.96-0.90 (m, 2H), 0.70-0.65 (m, 2H).

### Synthesis of 6-chloro-N-((6-cyclopropyl-3-fluoroimidazo[1,2-a]pyridin-2-yl)methyl)pyrimidin-4-amine

A mixture of 6-chloro-N-((6-cyclopropylimidazo[1,2-a]pyridin-2-yl)methyl)pyrimidin-4-amine (0.20 g, 0.67 mmol), Selectfluor™ (0.47 g, 1.3 mmol) and DMAP (0.082 g, 0.67 mmol) in CHCl₃ (5.0 mL) and water (1.0 mL) was stirred at 0° C. for 6 h. The mixture was warmed to room temperature and stirred for 18 h. The reaction was quenched with water and extracted with DCM. The organic layer was passed through a hydrophobic frit and concentrated in vacuo. The residue was purified by column chromatography on silica gel, eluting with 0-5% 7 N NH₃ in MeOH in DCM to give the title compound (0.042 g, 20%).

ESI-MS (M+H)+: 318.3, ¹H NMR (400 MHz, CDCl₃) δ 8.40 (s, 1H), 7.65 (d, J=0.8 Hz, 1H), 7.37-7.33 (m, 1H), 6.93 (dd, J=1.8, 9.4 Hz, 1H), 6.47 (s, 1H), 5.84 (s, 1H), 4.68 (s, 2H), 1.96-1.88 (m, 1H), 1.03-0.97 (m, 2H), 0.73-0.68 (m, 2H).

### Synthesis of 2-((2R,4S)-4-((tert-butyldimethylsilyl)oxy)pyrrolidin-2-yl)-6-cyclopropylimidazo[1,2-a]pyridine

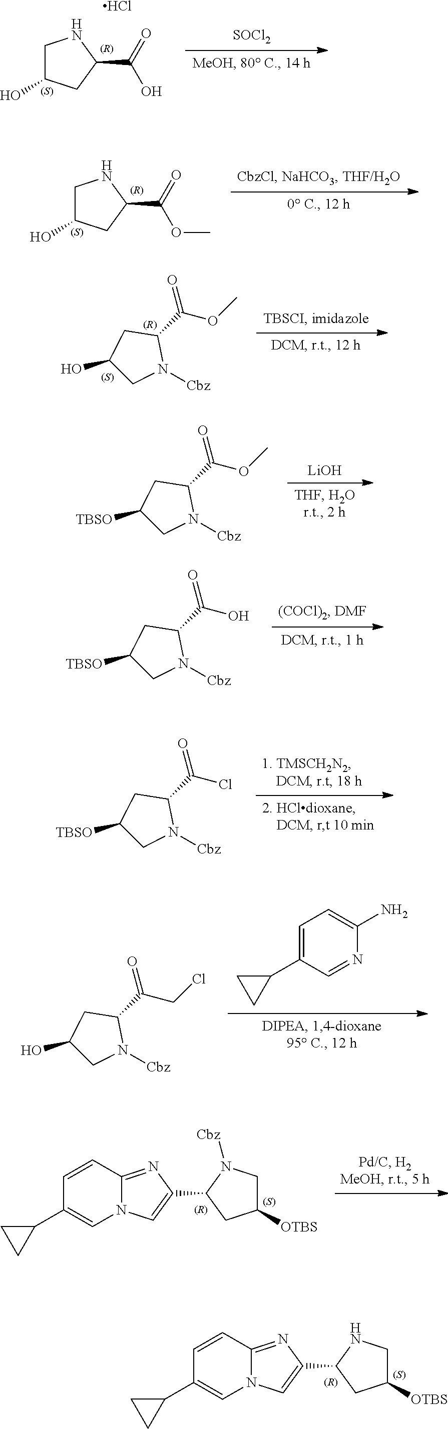

### Synthesis of methyl (2R,4S)-4-hydroxypyrrolidine-2-carboxylate

To a solution of (2R,4S)-4-hydroxypyrrolidine-2-carboxylic acid hydrochloride (5 g, 29.8 mmol) in MeOH (50 mL) was added SOCl₂ (10.6 g, 89.4 mmol) slowly at 0° C. After stirring at 80° C. for 12 h, the reaction was concentrated in vacuo to give methyl (2R,4S)-4-hydroxypyrrolidine-2-carboxylate (4 g, crude) as a grey solid. ESI-MS [M+H]+: 146.2.

### Synthesis of 1-benzyl 2-methyl (2R,4S)-4-hydroxypyrrolidine-1,2-dicarboxylate

To a solution of methyl (2R,4S)-4-hydroxypyrrolidine-2-carboxylate (4 g, 27.5 mmol) in THF (80 mL) was added saturated aqueous NaHCO₃ (60 ml), followed by CbzCl (5.6 g, 33.1 mmol) at 0° C. The reaction mixture was stirred at 0° C. for another 1 h and then extracted with EtOAc (3×50 mL). The combined organic layers were washed with brine, dried over Na₂SO₄ and concentrated in vacuo to give the crude, which was purified by silica gel chromatography (EtOAc/PE=1/5) to give 1-benzyl 2-methyl (2R,4s)-4-hydroxypyrrolidine-1,2-dicarboxylate (5.8 g, 75%) as yellow oil. ESI-MS [M+H]⁺: 280.1.

### Synthesis of 1-benzyl 2-methyl (2R,4S)-4-((tert-butyldimethylsilyl)oxy)pyrrolidine-1,2-dicarboxylate

A mixture of 1-benzyl 2-methyl (2R,4S)-4-hydroxypyrrolidine-1,2-dicarboxylate (4 g, 14.3 mmol), TMSCl (2.3 g, 21.5 mmol), imidazole (1.9 g, 28.6 mmol) in DCM (40 mL) was stirred at room temperature for 12 h. The reaction was washed with water (50 mL) then extracted with EtOAc (3×50 mL). The combined organic layers were washed with brine, dried over Na₂SO₄, filtered and concentrated in vacuo to give the crude, which was purified by silica gel chromatography (EtOAc/PE=1/3) to give 1-benzyl 2-methyl (2R,4S)-4-((tert-butyldimethylsilyl)oxy)pyrrolidine-1,2-dicarboxylate (4 g, 71%) as yellow oil. ESI-MS [M+H]⁺: 394.1.

### Synthesis of (2R,4S)-1-((benzyloxy)carbonyl)-4-((tert-butyldimethylsilyl)oxy)pyrrolidine-2-carboxylic acid

To a solution of 1-benzyl 2-methyl (2R,4S)-4-((tert-butyldimethylsilyl)oxy)pyrrolidine-1,2-dicarboxylate (4 g, 10.2 mmol) in THF (40 mL) was added LiOH (1.25 g, 30.6 mmol) and water (4 mL). The reaction mixture was stirred at room temperature for 2 h and then concentrated in vacuo to remove THF. The pH of the resulting residue was adjusted to 3-4 with HCl (1.0 M) then the mixture was extracted with EtOAc (3×50 mL). The combined organic layers were washed with brine, dried over Na₂SO₄, filtered and concentrated in vacuo to give the crude (2R,4S)-1-((benzyloxy)carbonyl)-4-((tert-butyldimethylsilyl)oxy)pyrrolidine-2-carboxylic acid (3.3 g, 87%) as yellow oil. ESI-MS [M+H]⁺: 394.1.

### Synthesis of benzyl (2R,4S)-4-((tert-butyldimethylsilyl)oxy)-2-(chlorocarbonyl)pyrrolidine-1-carboxylate

To a solution of (2R,4S)-1-((benzyloxy)carbonyl)-4-((tert-butyldimethylsilyl)oxy)pyrrolidine-2-carboxylic acid (2 g, crude from previous step) in DCM (20 mL) was added DMF (2 drops) and (COCl)₂ (1.0 g, 7.91 mmol) at 0° C. After stirring at room temperature for 1 h, the solution was concentrated to give benzyl (2R,4S)-4-((tert-butyldimethylsilyl)oxy)-2-(chlorocarbonyl)pyrrolidine-1-carboxylate (2.1 g crude) as yellow oil, which was used in next step without further purification. ESI-MS [M+H]⁺: 394.1.

### Synthesis of benzyl (2R,4S)-2-(2-chloroacetyl)-4-hydroxypyrrolidine-1-carboxylate

To a solution of benzyl (2R,4S)-4-((tert-butyldimethylsilyl)oxy)-2-(chlorocarbonyl)pyrrolidine-1-carboxylate (400 mg, 1 mmol) in DCM (20 mL) was added TMSCH₂N₂ (1.0 mL, 2M in hexane). After stirring at room temperature for 12 h, the solution was concentrated in vacuo to give the crude, which was re-dissolved in DCM (10 mL) and then HCl (4.0 M in 1,4-dioxane, 0.5 mL) was added. After stirring at room temperature for another 10 min, the solution was concentrated to give the crude, which was purified by silica gel chromatography (eluent: EtOAc/PE=1/5) to give benzyl (2R,4S)-2-(2-chloroacetyl)-4-hydroxypyrrolidine-1-carboxylate (150 mg, 36%) as yellow oil. ESI-MS [M+H]⁺: 298.2.

### Synthesis of benzyl (2R,4S)-4-((tert-butyldimethylsilyl)oxy)-2-(6-cyclopropylimidazo[1,2-a]pyridin-2-yl)pyrrolidine-1-carboxylate

A solution of benzyl (2R,4S)-2-(2-chloroacetyl)-4-hydroxypyrrolidine-1-carboxylate (150 mg, 0.51 mmol), 5-cyclopropylpyridin-2-amine (134 mg, 1.0 mmol) and DIPEA (329 mg, 2.55 mmol) in 1,4-dioxane (10 mL) was stirred at 95° C. for 12 h. The resulting mixture was concentrated in vacuo to give the crude, which was purified with silica gel chromatography (eluent: DCM/MeOH=15/1) to give benzyl (2R,4S)-4-((tert-butyldimethylsilyl)oxy)-2-(6-cyclopropylimidazo[1,2-a]pyridin-2-yl)pyrrolidine-1-carboxylate (150 mg, 58%) as a yellow solid. ESI-MS [M+H]⁺: 492.1.

### Synthesis of 2-((2R,4S)-4-((tert-butyldimethylsilyl)oxy)pyrrolidin-2-yl)-6-cyclopropylimidazo[1,2-a]pyridine

A mixture of benzyl (2R,4S)-4-((tert-butyldimethylsilyl)oxy)-2-(6-cyclopropylimidazo[1,2-a]pyridin-2-yl)pyrrolidine-1-carboxylate (150 mg, 0.31 mmol) and Pd/C (20 mg) in MeOH (10 mL) was stirred at room temperature for 5 h under a H₂ atmosphere. The reaction was filtered, washed with MeOH (30 mL). The filtrate was concentrated in vacuo to give 2-((2R,4S)-4-((tert-butyldimethylsilyl)oxy)pyrrolidin-2-yl)-6-cyclopropylimidazo[1,2-a]pyridine (120 mg, crude) as a yellow solid. ESI-MS [M+H]⁺: 358.2.

### Synthesis of 2-((2R,4R)-4-((tert-butyldimethylsilyl)oxy)pyrrolidin-2-yl)-6-cyclopropylimidazo[1,2-a]pyridine

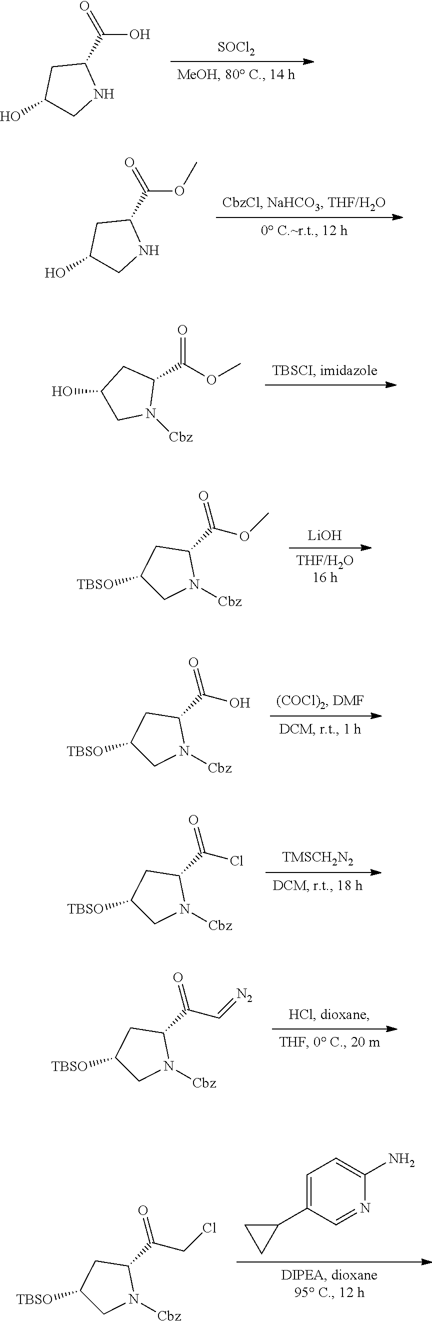

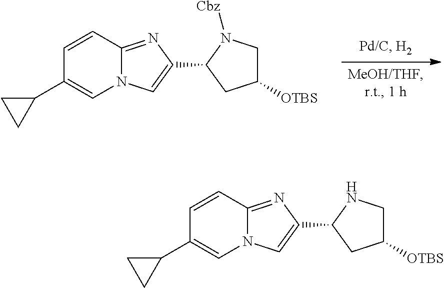 [SMILES: CC(C)(C)[Si](C)(C)O[C@@H]1C[C@H](c2cn3cc(C4CC4)ccc3n2)N(C(=O)OCc2ccccc2)C1 ; "MeOH/THF,r.t., 1 h" ; "Pd/C, H2" ; CC(C)(C)[Si](C)(C)O[C@H]1CN[C@@H](c2cn3cc(C4CC4)ccc3n2)C1]

Using a similar procedure to that for 2-((2R,4S)-4-((tert-butyldimethylsilyl)oxy)pyrrolidin-2-yl)-6-cyclopropylimidazo[1,2-a]pyridine, 2-((2R,4R)-4-((tert-butyldimethylsilyl)oxy)pyrrolidin-2-yl)-6-cyclopropylimidazo[1,2-a]pyridine (30 mg) was synthesised from (2R,4R)-4-hydroxypyrrolidine-2-carboxylic acid hydrochloric acid salt. ESI-MS [M+H]+: 358.2

### Synthesis of (R)-6-cyclopropyl-2-(pyrrolidin-2-yl)imidazo[1,2-a]pyridine

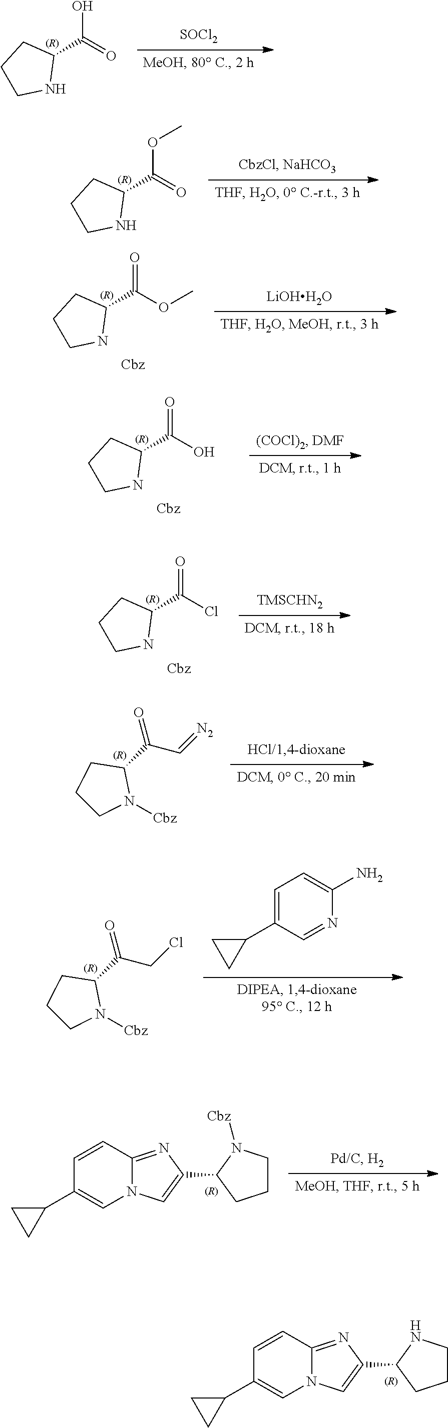

Using a similar procedure to that for 2-((2R,4S)-4-((tert-butyldimethylsilyl)oxy)pyrrolidin-2-yl)-6-cyclopropylimidazo[1,2-a]pyridine(R)-6-cyclopropyl-2-(pyrrolidin-2-yl)imidazo[1,2-a]pyridine (65 mg) was synthesised from D-proline. ESI-MS [M+H]+: 228.2

### Synthesis of 1-(2-((2R,4S)-4-((tert-butyldimethylsilyl)oxy)pyrrolidin-2-yl)-6-cyclopropylimidazo[1,2-a]pyridin-8-yl)-3-methylimidazolidine-2,4-dione

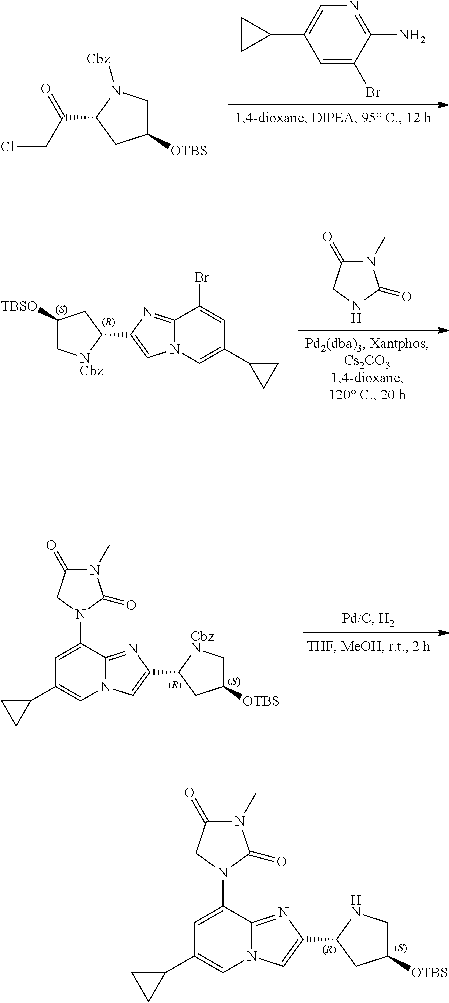

### Synthesis of benzyl (2R,4S)-2-(8-bromo-6-cyclopropylimidazo[1,2-a]pyridin-2-yl)-4-((tert-butyldimethylsilyl)oxy)pyrrolidine-1-carboxylate

A mixture of benzyl (2R,4S)-4-((tert-butyldimethylsilyl)oxy)-2-(2-chloroacetyl)pyrrolidine-1-carboxylate (0.55 g, 1.3 mmol), 3-bromo-5-cyclopropylpyridin-2-amine (0.51 g, 2.4 mmol) and DIPEA (0.52 g, 4.0 mmol) in dioxane (5.0 mL) was stirred at 95° C. for 16 h. After cooling to room temperature, the mixture was concentrated in vacuo to give the crude product, which was purified by column chromatography on silica gel (eluent: DCM/MeOH=10/1) to give benzyl (2R,4S)-2-(8-bromo-6-cyclopropylimidazo[1,2-a]pyridin-2-yl)-4-((tert-butyldimethylsilyl)oxy)pyrrolidine-1-carboxylate (0.20 g, 27%) as a yellow solid. ESI-MS [M+H]+: 572.1.

### Synthesis of benzyl (2R,4S)-4-((tert-butyldimethylsilyl)oxy)-2-(6-cyclopropyl-8-(3-methyl-2,4-dioxoimidazolidin-1-yl)imidazo[1,2-a]pyridin-2-yl)pyrrolidine-1-carboxylate

To a mixture of benzyl (2R,4S)-2-(8-bromo-6-cyclopropylimidazo[1,2-a]pyridin-2-yl)-4-((tert-butyldimethylsilyl)oxy)pyrrolidine-1-carboxylate (0.30 g, 0.53 mmol), 3-methylimidazolidine-2,4-dione (0.12 g, 1.1 mmol), and Cs₂CO₃ (0.52 g, 1.6 mmol) in dioxane (50 mL) was added Pd₂(dba)₃ (48 mg, 0.052 mmol) and Xantphos (61 mg, 0.11 mmol). The reaction mixture was stirred at 120° C. for 24 h under N₂. After cooling to room temperature, the mixture was filtered through Celite® and the filter cake was washed with DCM/MeOH (10/1, 100 mL). The filtrate was concentrated in vacuo to give the crude, which was purified by silica gel chromatography (eluent: DCM/MeOH=50/1˜20/1) to benzyl (2R,4S)-4-((tert-butyldimethylsilyl)oxy)-2-(6-cyclopropyl-8-(3-methyl-2,4-dioxoimidazolidin-1-yl)imidazo[1,2-a]pyridin-2-yl)pyrrolidine-1-carboxylate (0.31 g, 97%) as a yellow solid. ESI-MS [M+H]+: 604.1.

### Synthesis of 1-(2-((2R,4S)-4-((tert-butyldimethylsilyl)oxy)pyrrolidin-2-yl)-6-cyclopropylimidazo[1,2-a]pyridin-8-yl)-3-methylimidazolidine-2,4-dione

To a mixture of benzyl (2R,4S)-4-((tert-butyldimethylsilyl)oxy)-2-(6-cyclopropyl-8-(3-methyl-2,4-dioxoimidazolidin-1-yl)imidazo[1,2-a]pyridin-2-yl)pyrrolidine-1-carboxylate (0.31 g, 0.51 mmol) in THF (3.0 mL) and MeOH (3.0 mL) was added Pd/C (0.31 g). The mixture was stirred at room temperature for 2 h. The reaction mixture was filtered and concentrated in vacuo to give 1-(2-((2R,4S)-4-((tert-butyldimethylsilyl)oxy)pyrrolidin-2-yl)-6-cyclopropylimidazo[1,2-a]pyridin-8-yl)-3-methylimidazolidine-2,4-dione as a yellow solid (0.23 g, 96%), which was without further purification. ESI-MS [M+H]+: 470.1.

The compounds in Table A13 were synthesized using a similar method to 1-(2-((2R,4S)-4-((tert-butyldimethylsilyl)oxy)pyrrolidin-2-yl)-6-cyclopropylimidazo[1,2-a]pyridin-8-yl)-3-methylimidazolidine-2,4-dione starting from benzyl (2R,4S)-2-(8-bromo-6-cyclopropylimidazo[1,2-a]pyridin-2-yl)-4-((tert-butyldimethylsilyl)oxy)pyrrolidine-1-carboxylate and the appropriate coupling partner:

TABLE A13

| Structure | Coupling Partner | Analytical Data |
|---|---|---|
| 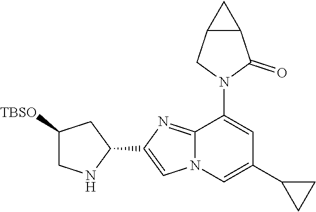 [SMILES: CC(C)(C)[Si](C)(C)O[C@@H]1CN[C@@H](c2cn3cc(C4CC4)cc(N4CC5CC5C4=O)c3n2)C1] | 3-azabicyclo[3.1.0] hexan-2-one | ESI-MS (M + H)+: 453.3 |
| 3-(2-((2RS,4RS)-4-((tert- | | |
| butyldimethylsilyl)oxy)pyrrolidine-2-yl)-6- | | |
| cyclopropylimidazo[1,2-a]pyridin-8-yl)-3- | | |
| azabicyclo[3.1.0]hexan-2-one | | |
| 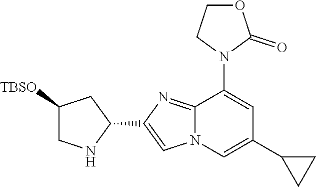 [SMILES: CC(C)(C)[Si](C)(C)O[C@@H]1CN[C@@H](c2cn3cc(C4CC4)cc(N4CCOC4=O)c3n2)C1] | oxazolidin-2-one | ESI-MS [M + H]+: 443.2 |
| 3-(2-((2R,4S)-4-((tert- | | |
| butyldimethylsilyl)oxy)pyrrolidin-2-yl)-6- | | |
| cyclopropylimidazo[1,2-a]pyridin-8- | | |
| yl)oxazolidin-2-one | | |
| 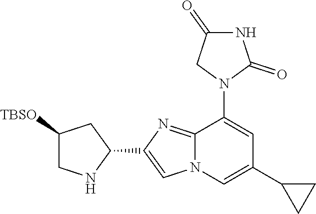 [SMILES: CC(C)(C)[Si](C)(C)O[C@@H]1CN[C@@H](c2cn3cc(C4CC4)cc(N4CC(=O)NC4=O)c3n2)C1] | imidazolidine-2,4- dione | ESI-MS [M + H]+: 456.2 |
| 1-(2-((2R,4S)-4-((tert- | | |
| butyldimethylsilyl)oxy)pyrrolidin-2-yl)-6- | | |
| cyclopropylimidazo[1,2-a]pyridin-8- | | |
| yl)imidazolidine-2,4-dione | | |

### Synthesis of (3S,5R)-5-(6-cyclopropyl-8-(3-methyl-2,4-dioxoimidazolidin-1-yl)imidazo[1,2-a]pyridin-2 yl)pyrrolidin-3 yl propionate

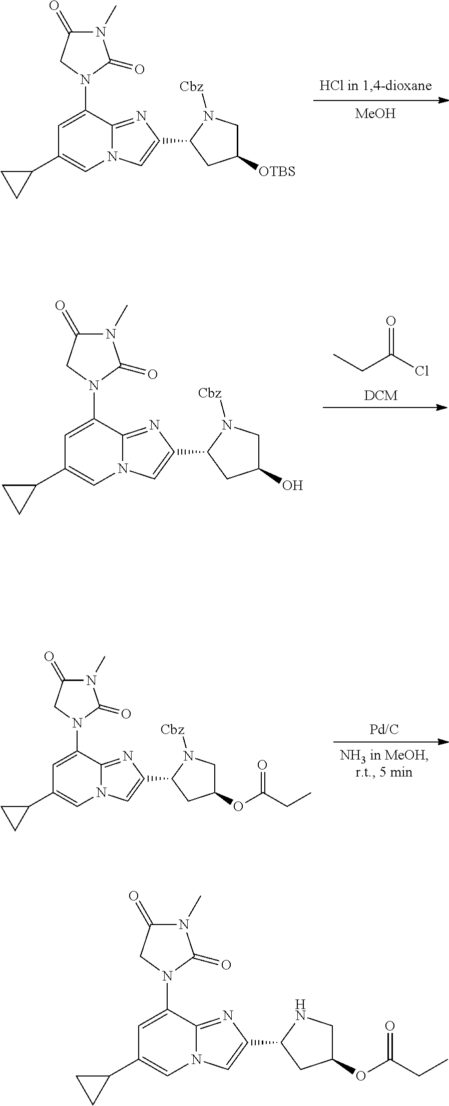

### Synthesis of benzyl (2R,4S)-2-(6-cyclopropyl-8-(3-methyl-2,4-dioxoimidazolidin-1-yl)imidazo[1,2-a]pyridin-2-yl)-4-hydroxypyrrolidine-1-carboxylate

To a mixture of benzyl (2R,4S)-4-((tert-butyldimethylsilyl)oxy)-2-(6-cyclopropyl-8-(3-methyl-2,4-dioxoimidazolidin-1-yl)imidazo[1,2-a]pyridin-2-yl)pyrrolidine-1-carboxylate (210 mg, 0.35 mmol) in MeOH (2 mL) was added HCl (4M solution in dioxane, 2 mL). The mixture was stirred at 25° C. for 2 h then concentrated in vacuo to give benzyl (2R,4S)-2-(6-cyclopropyl-8-(3-methyl-2,4-dioxoimidazolidin-1-yl)imidazo[1,2-a]pyridin-2-yl)-4-hydroxypyrrolidine-1-carboxylate (170 mg, crude) as a yellow solid. ESI-MS [M+H]+: 490.2.

### Synthesis of benzyl (2R,4S)-2-(6-cyclopropyl-8-(3-methyl-2,4-dioxoimidazolidin-1-yl)imidazo[1,2-a]pyridin-2-yl)-4-(propionyloxy)pyrrolidine-1-carboxylate

To a mixture of benzyl (2R,4S)-2-(6-cyclopropyl-8-(3-methyl-2,4-dioxoimidazolidin-1-yl)imidazo[1,2-a]pyridin-2-yl)-4-hydroxypyrrolidine-1-carboxylate (150 mg, 0.3 mmol) in DCM (1 mL) was added propionyl chloride (1 mL) and the mixture was stirred at 25° C. for 2 h under N₂. The mixture was basified with saturated aq·NaHCO₃ solution to pH 8 and then extracted with (DCM (3×20 mL). The combined organic layers were washed with brine (20 mL), dried over anhydrous Na₂SO₄, and concentrated in vacuo to give the crude product, which was purified by silica gel chromatography (eluent: PE/EtOAc=2/1) to give benzyl (2R,4S)-2-(6-cyclopropyl-8-(3-methyl-2,4-dioxoimidazolidin-1-yl)imidazo[1,2-a]pyridin-2-yl)-4-(propionyloxy)pyrrolidine-1-carboxylate (120 mg, 72%) as a white solid. ESI-MS [M+H]⁺: 546.2.

### Synthesis of (3S,5R)-5-(6-cyclopropyl-8-(3-methyl-2,4-dioxoimidazolidin-1-yl)imidazo[1,2-a]pyridin-2-yl)pyrrolidin-3-yl propionate

To a mixture of benzyl (2R,4S)-2-(6-cyclopropyl-8-(3-methyl-2,4-dioxoimidazolidin-1-yl)imidazo[1,2-a]pyridin-2-yl)-4-(propionyloxy)pyrrolidine-1-carboxylate (100 mg, 0.18 mmol) in NH₃ (7M in MeOH, 2 mL) was added Pd/C (100 mg) and the mixture was stirred at 25° C. for 5 min under H₂. The mixture was filtered and the filtrate was concentrated in vacuo to give (3S,5R)-5-(6-cyclopropyl-8-(3-methyl-2,4-dioxoimidazolidin-1-yl)imidazo[1,2-a]pyridin-2-yl)pyrrolidin-3-yl propionate (60 mg, 82%) as a white solid. ESI-MS [M+H]⁺: 412.2

### Synthesis of (5R)-5-(6-cyclopropylimidazo[1,2-a]pyridin-2-yl)-3-methylpyrrolidin-3-ol

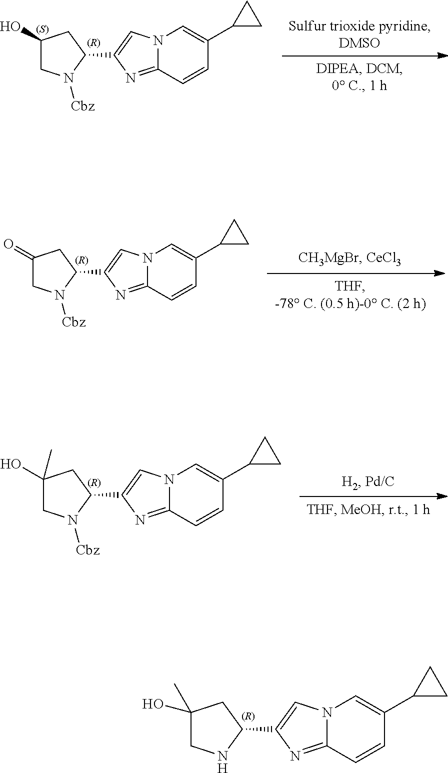

### Synthesis of benzyl (R)-2-(6-cyclopropylimidazo[1,2-a]pyridin-2-yl)-4-oxopyrrolidine-1-carboxylate

To a mixture of benzyl (2R,4S)-2-(6-cyclopropylimidazo[1,2-a]pyridin-2-yl)-4-hydroxypyrrolidine-1-carboxylate (0.55 g, 1.5 mmol) in DCM (6 mL) was added DIPEA (0.73 mL, 4.4 mmol) dropwise at 0° C. followed by sulfur trioxide pyridine (1.2 g, 7.4 mmol) in DMSO (3.0 mL) dropwise at 0° C. After stirring at 0° C. for 1 h, the mixture was quenched with water (10 mL) and extracted with DCM (3×50 mL). The organic layers were combined and washed with brine (10 mL). The mixture was concentrated in vacuo and purified by column chromatography on silica gel (eluent: DCM/MeOH=20/1) to give benzyl (R)-2-(6-cyclopropylimidazo[1,2-a]pyridin-2-yl)-4-oxopyrrolidine-1-carboxylate (0.52 mg, 95%) as a yellow oil. ESI-MS [M+H]⁺: 376.2.

### Synthesis of benzyl (2R)-2-(6-cyclopropylimidazo[1,2-a]pyridin-2-yl)-4-hydroxy-4-methylpyrrolidine-1-carboxylate

A mixture of CeCl₃ (0.80 g, 3.2 mmol) in THF (12 mL) was added CH₃MgBr (3.4 mL, 10 mmol, 3 mol/L in diethyl ether) at −78° C. and stirred for 0.5 h. Then to the resulting the mixture was added benzyl (R)-2-(6-cyclopropylimidazo[1,2-a]pyridin-2-yl)-4-oxopyrrolidine-1-carboxylate (0.48 g, 1.3 mmol) in THF (3.0 mL) at −78° C. After stirring at 0° C. for 2 h, the mixture was quenched with a solution of saturated aq·NH₄Cl (10 mL) and extracted with EtOAc (3×60 mL). The organic layers were combined and washed with brine (10 mL) and concentrated in vacuo. The residue was purified by column chromatography on silica gel (eluent: DCM/MeOH=20/1) to give benzyl (2R)-2-(6-cyclopropylimidazo[1,2-a]pyridin-2-yl)-4-hydroxy-4-methylpyrrolidine-1-carboxylate (0.50 g, 98%) as a yellow oil. ESI-MS [M+H]⁺: 392.2.

### Synthesis of (5R)-5-(6-cyclopropylimidazo[1,2-a]pyridin-2-yl)-3-methylpyrrolidin-3-ol

A mixture of benzyl (2R)-2-(6-cyclopropylimidazo[1,2-a]pyridin-2-yl)-4-hydroxy-4-methylpyrrolidine-1-carboxylate (0.50 g, 1.3 mmol) in THF (2.5 mL) and MeOH (2.5 mL) was added 10% Pd on carbon (0.50 g) at room temperature. The mixture was stirred at room temperature under and atmosphere of H₂ for 1 h. The mixture was filtered and concentrated in vacuo to give (5R)-5-(6-cyclopropylimidazo[1,2-a]pyridin-2-yl)-3-methylpyrrolidin-3-ol (0.14 g, crude) as a yellow oil. ESI-MS [M+H]⁺: 258.2.

### Synthesis of ((3S,5R)-5-(6-cyclopropylimidazo[1,2-a]pyridin-2-yl)pyrrolidin-3-yl)methanol

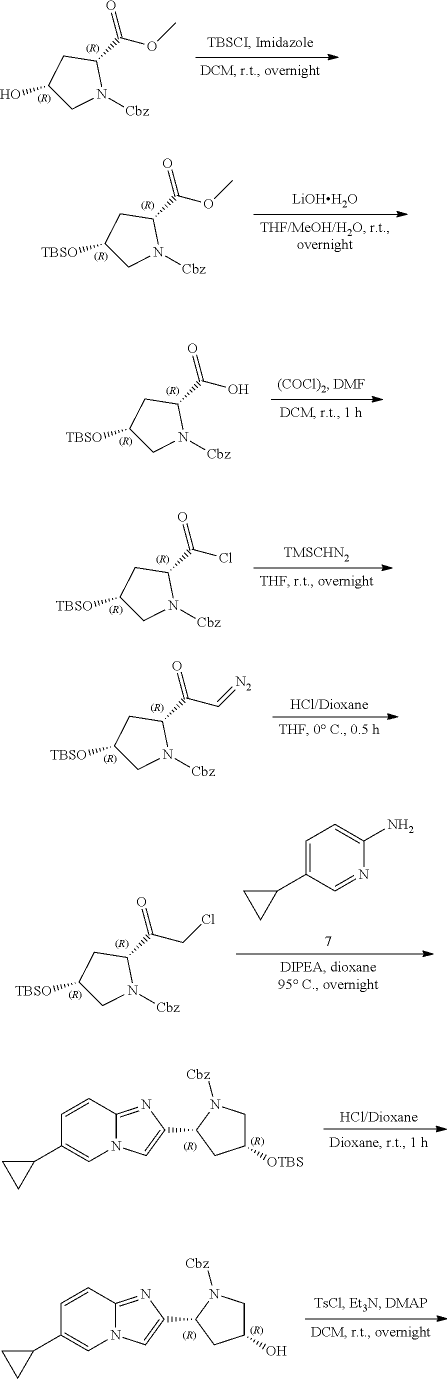

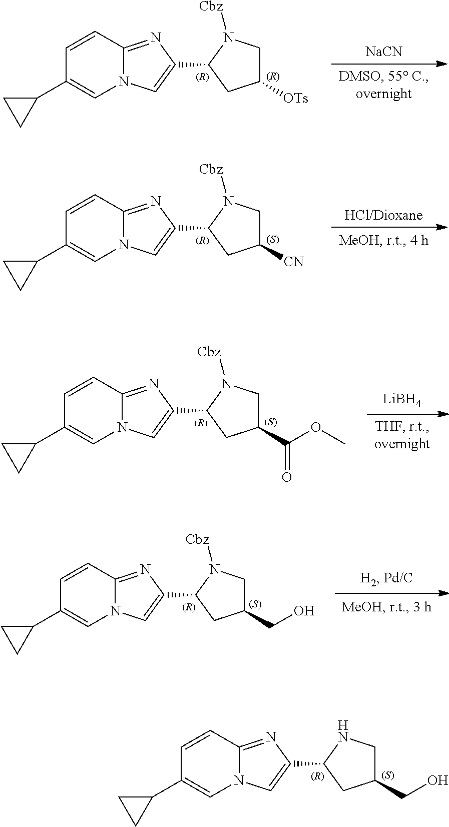

### Synthesis of benzyl (2R,4R)-2-(6-cyclopropylimidazo[1,2-a]pyridin-2-yl)-4-(tosyloxy)pyrrolidine-1-carboxylate

Using a similar procedure to that for benzyl (2R,4S)-4-((tert-butyldimethylsilyl)oxy)-2-(6-cyclopropylimidazo[1,2-a]pyridin-2-yl)pyrrolidine-1-carboxylate, benzyl (2R,4R)-4-((tert-butyldimethylsilyl)oxy)-2-(6-cyclopropylimidazo[1,2-a]pyridin-2-yl)pyrrolidine-1-carboxylate (1.1 g) was synthesised 1-benzyl 2-methyl (2R,4R)-4-hydroxypyrrolidine-1,2-dicarboxylate (5.0 g, 18 mmol).

### Synthesis of benzyl (2R,4R)-2-(6-cyclopropylimidazo[1,2-a]pyridin-2-yl)-4-hydroxypyrrolidine-1-carboxylate

To a mixture of benzyl (2R,4R)-4-((tert-butyldimethylsilyl)oxy)-2-(6-cyclopropylimidazo[1,2-a]pyridin-2-yl)pyrrolidine-1-carboxylate (1.1 g, 2.2 mmol) in 1,4-dioxane (10 mL) was added HCl (10 mL, 40 mmol, 4 mol/L in 1,4-dioxane) at room temperature. After stirring at room temperature for 1 h, the mixture was concentrated in vacuo and the residue was basified with saturated aq·NaHCO₃ (30 mL) to pH=8. The mixture was extracted with DCM (3×60 mL). The organic layers were combined and concentrated in vacuo to give benzyl (2R,4R)-2-(6-cyclopropylimidazo[1,2-a]pyridin-2-yl)-4-hydroxypyrrolidine-1-carboxylate (0.91 g, crude) as a brown oil, which was used without purification. ESI-MS [M+H]⁺: 378.2.

### Synthesis of benzyl (2R,4R)-2-(6-cyclopropylimidazo[1,2-a]pyridin-2-yl)-4-(tosyloxy)pyrrolidine-1-carboxylate

To a mixture of benzyl (2R,4R)-2-(6-cyclopropylimidazo[1,2-a]pyridin-2-yl)-4-hydroxypyrrolidine-1-carboxylate (0.91 g, 2.4 mmol), TsCl (1.4 g, 7.2 mmol) and Et₃N (1.0 mL, 7.2 mmol) in DCM (10 mL) was added DMAP (0.15 g, 1.2 mmol) at room temperature. After stirring at room temperature overnight, the mixture was quenched with water (20 mL) and extracted with DCM (3×60 mL). The combined organic layers were concentrated in vacuo and purified by column chromatography on silica gel (eluent: DCM/MeOH=10/1) to give benzyl (2R,4R)-2-(6-cyclopropylimidazo[1,2-a]pyridin-2-yl)-4-(tosyloxy)pyrrolidine-1-carboxylate (0.78 g, 61%) as a yellow oil. ESI-MS [M+H]⁺: 532.2.

### Synthesis of benzyl (2R,4S)-4-cyano-2-(6-cyclopropylimidazo[1,2-a]pyridin-2-yl)pyrrolidine-1-carboxylate

A mixture of benzyl (2R,4R)-2-(6-cyclopropylimidazo[1,2-a]pyridin-2-yl)-4-(tosyloxy)pyrrolidine-1-carboxylate (0.78 g, 1.5 mmol) in DMSO (10 mL) was added NaCN (0.29 g, 5.9 mmol) at room temperature. After stirring at 55° C. overnight, the mixture was washed with water (30 mL) and extracted with EtOAc (3×60 mL). The organic layers were combined and purified by column chromatography on silica gel (eluent: DCM/MeOH=15/1) to give benzyl (2R,4S)-4-cyano-2-(6-cyclopropylimidazo[1,2-a]pyridin-2-yl)pyrrolidine-1-carboxylate (0.52 g, 91%) as an orange oil. ESI-MS [M+H]⁺: 387.2.

### Synthesis of 1-benzyl 3-methyl (3S,5R)-5-(6-cyclopropylimidazo[1,2-a]pyridin-2-yl)pyrrolidine-1,3-dicarboxylate

A mixture of benzyl (2R,4S)-4-cyano-2-(6-cyclopropylimidazo[1,2-a]pyridin-2-yl)pyrrolidine-1-carboxylate (0.35 g, 0.90 mmol) in MeOH (4.0 mL) was added HCl (7.0 mL, 28 mmol, 4 M in 1,4-dioxane) was stirred at room temperature for 4 h. The mixture was concentrated in vacuo and then the residue was diluted with saturated aq·NaHCO₃ (10 mL). The aqueous was extracted with EtOAc (3×30 mL). The combined organic layers were concentrated in vacuo and purified by preparative TLC (eluent: DCM/MeOH=40/1) to give 1-benzyl 3-methyl (3S,5R)-5-(6-cyclopropylimidazo[1,2-a]pyridin-2-yl)pyrrolidine-1,3-dicarboxylate (0.21 g, 55%) as a yellow oil. ESI-MS [M+H]⁺: 420.2.

### Synthesis of benzyl (2R,4S)-2-(6-cyclopropylimidazo[1,2-a]pyridin-2-yl)-4-(hydroxymethyl)pyrrolidine-1-carboxylate

To a mixture of 1-benzyl 3-methyl (3S,5R)-5-(6-cyclopropylimidazo[1,2-a]pyridin-2-yl)pyrrolidine-1,3-dicarboxylate (0.13 g, 0.31 mmol) in THF (3.0 mL) was added LiBH₄ (14 mg, 0.62 mmol) at 0° C. After stirring at room temperature overnight, the mixture was quenched with saturated aq·NH₄Cl (30 mL) and extracted with EtOAc (3×60 mL). The organic layers were combined and concentrated in vacuo to give benzyl (2R,4S)-2-(6-cyclopropylimidazo[1,2-a]pyridin-2-yl)-4-(hydroxymethyl)pyrrolidine-1-carboxylate (69 mg, crude) as a white solid, which was used without purification. ESI-MS [M+H]⁺: 392.2.

### Synthesis of ((3S,5R)-5-(6-cyclopropylimidazo[1,2-a]pyridin-2-yl)pyrrolidin-3-yl)methanol

To a mixture of benzyl (2R,4S)-2-(6-cyclopropylimidazo[1,2-a]pyridine-2-yl)-4-(hydroxymethyl) pyrrolidine-1-carboxylate (69 mg, 0.18 mmol) in MeOH (2.0 mL) was added 10% Pd on carbon (70 mg) at room temperature. The mixture was stirred for 3 h under an atmosphere of H₂. The mixture was filtered and concentrated in vacuo to give ((3S,5R)-5-(6-cyclopropylimidazo[1,2-a]pyridin-2-yl)pyrrolidin-3-yl)methanol (37 mg, crude) as a yellow oil, which was used without purification. ESI-MS [M+H]⁺: 258.2.

### Synthesis of 2-((2S,3S,4R)-3,4-bis((tert-butyldimethylsilyl)oxy)pyrrolidin-2-yl)-6-cyclopropylimidazo[1,2-a]pyridine

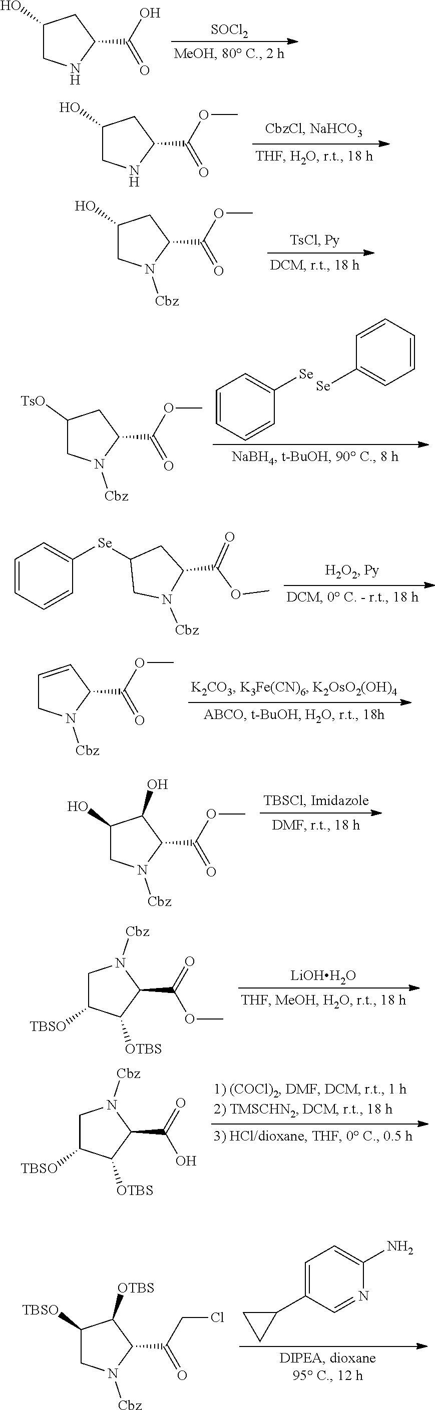

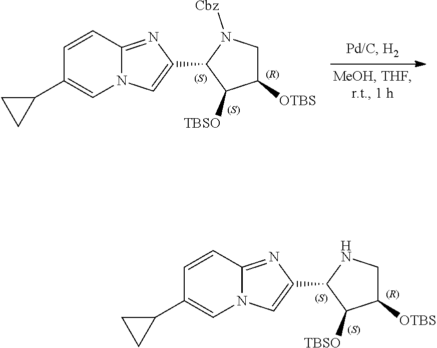

### Synthesis of methyl (2R,4R)-4-hydroxypyrrolidine-2-carboxylate

To a mixture of (2R,4R)-4-hydroxypyrrolidine-2-carboxylic acid (25 g, 0.19 mol) in MeOH (0.25 L) was added SOCl₂ (25 mL) at 0° C. The reaction mixture was then warmed to 80° C. and stirred for 2 h. After cooling to room temperature, the mixture was concentrated in vacuo to afford methyl (2R,4R)-4-hydroxypyrrolidine-2-carboxylate (35 g, crude) as a white solid, which was used without further purification. ESI-MS [M+H]+: 146.1

### Synthesis of 1-benzyl 2-methyl (2R,4R)-4-hydroxypyrrolidine-1,2-dicarboxylate

To a mixture of methyl (2R,4R)-4-hydroxypyrrolidine-2-carboxylate (35 g, 0.2 mol) and NaHCO₃ (48 g, 0.57 mol) in THF/H₂O (0.60 L/0.40 L) was added CbzCl (49 g, 0.29 mol) at 0° C. The mixture was then warmed to room temperature and stirred for 18 h. The resulting solution was extracted with EtOAc (3×0.30 L). The combined organic layers were washed with brine (0.30 L), dried over Na₂SO₄ and concentrated in vacuo. The residue was purified by column chromatography on silica gel (eluent: EtOAc/PE=0-100%) to afford 1-benzyl 2-methyl (2R,4R)-4-hydroxypyrrolidine-1,2-dicarboxylate (50 g, 94% for two steps) as a white solid. ESI-MS [M+H]+: 280.1

### Synthesis of 1-benzyl 2-methyl (2R)-4-(tosyloxy)pyrrolidine-1,2-dicarboxylate

A mixture of 1-benzyl 2-methyl (2R,4R)-4-hydroxypyrrolidine-1,2-dicarboxylate (15 g, 54 mmol), TsCl (46 g, 81 mmol) and DMAP (20 g, 0.16 mol) in DCM (0.20 L) was stirred at room temperature for 18 h. The mixture was quenched with water (0.50 L), and then extracted with DCM (3×0.30 L). The combined organic layers were washed with brine (0.20 L), dried over Na₂SO₄ and concentrated in vacuo. The residue was purified by column chromatography on silica gel (eluent: EtOAc/PE=0-30%) to afford 1-benzyl 2-methyl (2R)-4-(tosyloxy)pyrrolidine-1,2-dicarboxylate (19 g, 82%) as a white solid. ESI-MS [M+H]+: 434.1

### Synthesis of 1-benzyl 2-methyl (2R)-4-(phenylselanyl)pyrrolidine-1,2-dicarboxylate

To a mixture of 1,2-diphenyldiselane (2.6 g, 8.3 mmol) in t-BuOH (0.10 L) was added NaBH₄ (0.63 g, 17 mmol) and 1-benzyl 2-methyl (2R)-4-(tosyloxy)pyrrolidine-1,2-dicarboxylate (6.0 g, 14 mmol) and the mixture was stirred at 90° C. for 8 h. The mixture was quenched with water (0.20 L), filtered then extracted with DCM (3×0.10 L). The combined organic layers were washed with brine (0.10 L), dried over Na₂SO₄ and concentrated in vacuo. The residue was purified by column chromatography on silica gel (eluent: EtOAc/PE=0-30%) to afford 1-benzyl 2-methyl (2R)-4-(phenylselanyl)pyrrolidine-1,2-dicarboxylate (4 g, 68%) as a colorless oil. ESI-MS [M+H]+: 420.1

### Synthesis of 1-benzyl 2-methyl (R)-2,5-dihydro-1H-pyrrole-1,2-dicarboxylate

To the mixture of 1-benzyl 2-methyl (2R)-4-(phenylselanyl)pyrrolidine-1,2-dicarboxylate (2.1 g, 5.0 mmol) and pyridine (0.56 mL, 7.0 mmol) in DCM (30 mL) was added H₂O₂ (1.4 mL, 13 mmol, 30% in water) at 0° C. and the mixture was stirred at room temperature for 18 h. The mixture was quenched with saturated aq·Na₂SO₃ (30 mL) and extracted with DCM (3×50 mL). The combined organic layers were washed with brine (30 mL), dried over Na₂SO₄ and concentrated in vacuo. The residue was purified by column chromatography on silica gel (eluent: EtOAc/PE=0-30%) to afford 1-benzyl 2-methyl (R)-2,5-dihydro-1H-pyrrole-1,2-dicarboxylate (1.0 g, 77%) as a colorless oil. ESI-MS [M+H]+: 262.1

### Synthesis of 1-benzyl 2-methyl (2R,3S,4R)-3,4-dihydroxypyrrolidine-1,2-dicarboxylate

To the mixture of 1-benzyl 2-methyl (R)-2,5-dihydro-1H-pyrrole-1,2-dicarboxylate (6.5 g, 25 mmol), K₃Fe(CN)₆ (25 g, 75 mmol), 1-azabicyclo[2.2.2]octane (0.13 g, 1.2 mmol) and K₂CO₃ (10 g, 75 mmol) in t-BuOH/H₂O (80 mL/80 mL) was added K₂OsO₂(OH)₄ (1.1 g, 1.2 mmol). The mixture was stirred at room temperature for 18 h. then quenched with saturated aq·Na₂SO₃ (30 mL) and extracted with EA (3×200 mL). The combined organic layers were washed with brine (200 mL), dried over Na₂SO₄ and concentrated in vacuo. The residue was purified by column chromatography on silica gel (eluent: MeOH/DCM=0-5%) to afford 1-benzyl 2-methyl (2R,3S,4R)-3,4-dihydroxypyrrolidine-1,2-dicarboxylate (5.5 g, 75%) as a colorless oil. ESI-MS [M+H]+: 296.1.

### Synthesis of 1-benzyl 2-methyl (2R,3S,4R)-3,4-bis((tert-butyldimethylsilyl)oxy)pyrrolidine-1,2-dicarboxylate

To a mixture of 1-benzyl 2-methyl (2R,3S,4R)-3,4-dihydroxypyrrolidine-1,2-dicarboxylate (5.2 g, 18 mmol) and imidazole (7.2 g, 0.11 mol) in DMF (50 mL) was added TBSCl (7.9 g, 53 mmol). The mixture was stirred at room temperature for 18 h then quenched with water (500 mL), and then extracted with EA (3×300 mL). The combined organic layers were washed with brine (200 mL), dried over Na₂SO₄ and concentrated in vacuo. The residue was purified by column chromatography on silica gel (eluent: EtOAc/PE=0-15%) to afford 1-benzyl 2-methyl (2R,3S,4R)-3,4-bis((tert-butyldimethylsilyl)oxy)pyrrolidine-1,2-dicarboxylate (8.2 g, 89%) as a colorless oil. ESI-MS [M+H]+: 524.3

### Synthesis of 2-((2S,3S,4R)-3,4-bis((tert-butyldimethylsilyl)oxy)pyrrolidin-2-yl)-6-cyclopropylimidazo[1,2-a]pyridine

Using a similar procedure to that for 2-((2R,4S)-4-((tert-butyldimethylsilyl)oxy)pyrrolidin-2-yl)-6-cyclopropylimidazo[1,2-a]pyridine, 2-((2S,3S,4R)-3,4-bis((tert-butyldimethylsilyl)oxy)pyrrolidin-2-yl)-6-cyclopropylimidazo[1,2-a]pyridine (0.10 g) was synthesised from 1-benzyl 2-methyl (2R,3S,4R)-3,4-bis((tert-butyldimethylsilyl)oxy)pyrrolidine-1,2-dicarboxylate. ESI-MS [M+H]+: 488.3

### Synthesis of 2-((2R,4S)-4-((tert-butyldimethylsilyl)oxy)pyrrolidin-2-yl)-6-(2-methylcyclopropyl)imidazo[1,2-a]pyridine

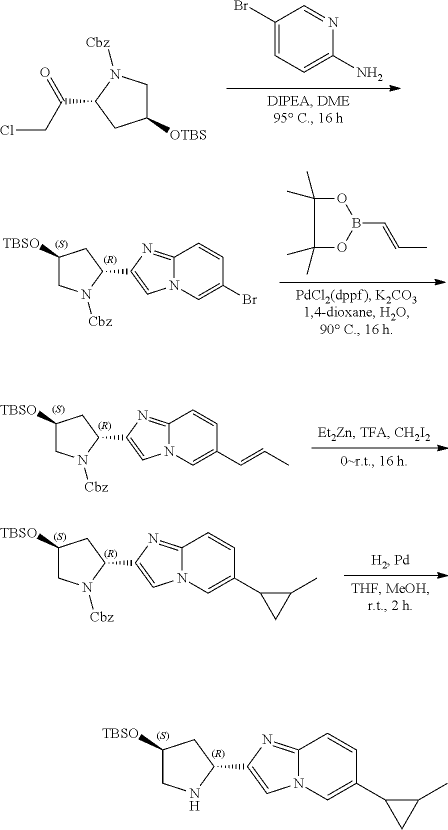

### Synthesis of benzyl (2R,4S)-2-(6-bromoimidazo[1,2-a]pyridin-2-yl)-4-((tert-butyldimethylsilyl)oxy)pyrrolidine-1-carboxylate

A mixture of benzyl (2R,4S)-4-((tert-butyldimethylsilyl)oxy)-2-(2-chloroacetyl)pyrrolidine-1-carboxylate (1.0 g, 2.4 mmol), 5-bromopyridin-2-amine (0.83 g, 4.8 mmol) and DIPEA (0.93 g, 7.2 mmol) in DME (30 mL) was stirred at 95° C. for 16 h. The resulting mixture was concentrated in vacuo and then extracted with DCM (3×50 mL). The combined organic layers were concentrated in vacuo to get the crude, which was purified by column chromatography (eluent: EA/PE=1/2) to give benzyl (2R,4S)-2-(6-bromoimidazo[1,2-a]pyridin-2-yl)-4-((tert-butyldimethylsilyl)oxy)pyrrolidine-1-carboxylate (0.20 g, 16%) as a yellow oil. ESI-MS [M+H]+: 530.2.

### Synthesis of benzyl (2R,4S)-4-((tert-butyldimethylsilyl)oxy)-2-(6-((E)-prop-1-en-1-yl)imidazo[1,2-a]pyridin-2-yl)pyrrolidine-1-carboxylate

A mixture of benzyl (2R,4S)-2-(6-bromoimidazo[1,2-a]pyridin-2-yl)-4-((tert-butyldimethylsilyl)oxy)pyrrolidine-1-carboxylate (0.50 g, 0.94 mmol), PdCl₂(dppf) (0.14 g, 0.19 mmol), (E)-4,4,5,5-tetramethyl-2-(prop-1-en-1-yl)-1,3,2-dioxaborolane (0.47 g, 2.8 mmol) and K₂CO₃ (0.39 g, 2.8 mmol) in s solution of 1,4-dioxane (10 mL) and water (2.0 mL) was stirred at 95° C. under N₂ for 16 h. The reaction mixture was quenched with water (50 mL) and extracted with EtOAc (5×70 mL). The combined organic layers were washed with brine, dried over Na₂SO₄, and concentrated in vacuo to give the crude, which was purified with column chromatography (eluent: PE/EtOAc=1/1) to give benzyl (2R,4S)-4-((tert-butyldimethylsilyl)oxy)-2-(6-((E)-prop-1-en-1-yl)imidazo[1,2-a]pyridin-2-yl)pyrrolidine-1-carboxylate (0.33 g, 71%) as a white solid. ESI-MS [M+H]+: 492.2.

### Synthesis of benzyl (2R,4S)-4-((tert-butyldimethylsilyl)oxy)-2-(6-(2-methylcyclopropyl)imidazo[1,2-a]pyridin-2-yl)pyrrolidine-1-carboxylate

To a solution of DCM (0.5 mL) was added Et₂Zn (0.92 mL, 1.83 mmol, 2.0 M in hexanes) under N₂ atmosphere. The solution was cooled in an ice bath and a solution of trifluoroacetic acid (209 mg, 1.83 mmol) in DCM (1.0 mL) was then dripped very slowly into the reaction mixture via syringe. Upon stirring for 10 min, a solution of CH₂l₂ (490 mg, 1.83 mmol) in DCM (1.0 mL) was added. After an additional 20 min of stirring, a solution of benzyl (2R,4S)-4-((tert-butyldimethylsilyl)oxy)-2-(6-((E)-prop-1-en-1-yl)imidazo[1,2-a]pyridin-2-yl)pyrrolidine-1-carboxylate (90 mg, 0.1 mmol) in DCM (1.0 mL) was added, and the ice bath was removed. After the reaction mixture was stirred at room temperature for 16 h, the mixture was quenched with saturated aq·NH₄Cl (10 mL) and the phases were separated. The aqueous layer was extracted with DCM (2×10 mL). The combined organic layers were concentrated in vacuo and purified by Prep-HPLC (eluent: DCM/MeOH=10/1) to give benzyl (2R,4S)-4-((tert-butyldimethylsilyl)oxy)-2-(6-(2-methylcyclopropyl)imidazo[1,2-a]pyridin-2-yl)pyrrolidine-1-carboxylate (10 mg, 11%) as a colourless oil. ESI-MS [M+H]+: 506.2.

### Synthesis of 2-((2R,4S)-4-((tert-butyldimethylsilyl)oxy)pyrrolidin-2-yl)-6-(2-methylcyclopropyl)imidazo[1,2-a]pyridine

To a mixture of benzyl (2R,4S)-4-((tert-butyldimethylsilyl)oxy)-2-(6-(2-methylcyclopropyl)imidazo[1,2-a]pyridin-2-yl)pyrrolidine-1-carboxylate (10 mg, 0.02 mmol) in THF (1 mL) and MeOH (1 mL) was added Pd/C (10 mg). the mixture was stirred at room temperature under the H₂ for 2 h. The reaction mixture was filtered through Celite® and the filter cake was washed with DCM/MeOH (10/1, 10 mL). The filtrate was concentrated in vacuo to give 2-((2R,4S)-4-((tert-butyldimethylsilyl)oxy)pyrrolidin-2-yl)-6-(2-methylcyclopropyl)imidazo[1,2-a]pyridine (6 mg, 81%) as a yellow oil. ESI-MS [M+H]+: 372.2.

### Synthesis of 6-cyclopropyl-2-((2R,3S)-3-((triisopropylsilyl)oxy)pyrrolidin-2-yl)imidazo[1,2-a]pyridine

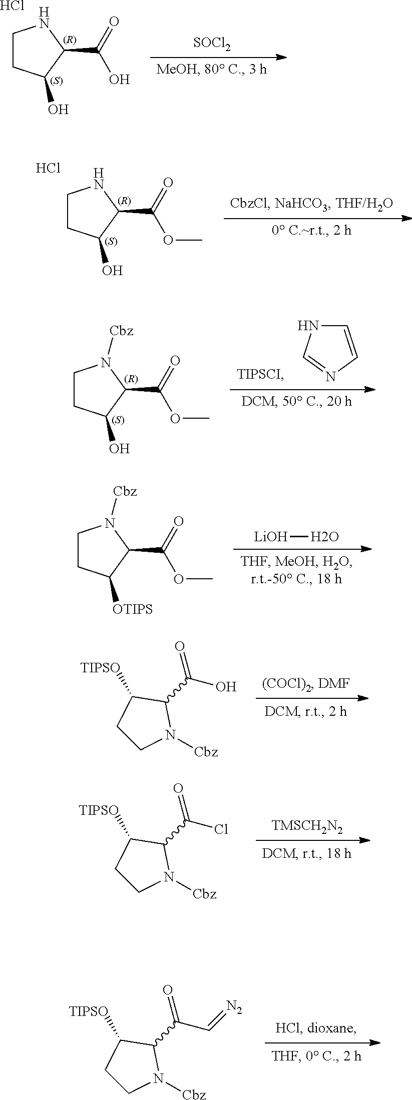

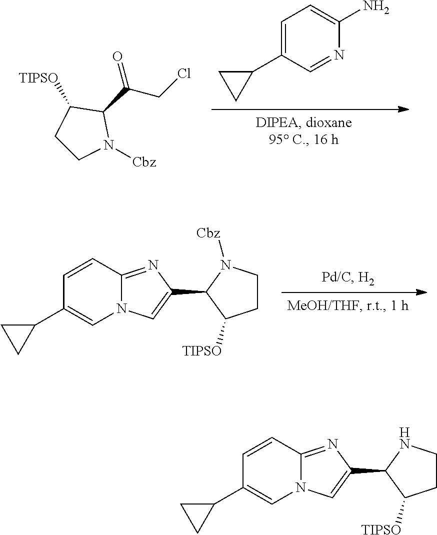

Using a similar procedure to that for 2-((2R,4S)-4-((tert-butyldimethylsilyl)oxy)pyrrolidin-2-yl)-6-cyclopropylimidazo[1,2-a]pyridine, 6-cyclopropyl-2-((2R,3S)-3-((triisopropylsilyl)oxy)pyrrolidin-2-yl)imidazo[1,2-a]pyridine (35 mg) was synthesised from (2R,3S)-3-hydroxypyrrolidine-2-carboxylic acid hydrochloric acid salt. TIPSCl was used in place of TBSCl. ESI-MS [M+H]⁺:400.3

### Synthesis of 6-cyclopropyl-2-((2S,3R)-3-((triisopropylsilyl)oxy)pyrrolidin-2-yl)imidazo[1,2-a]pyridine

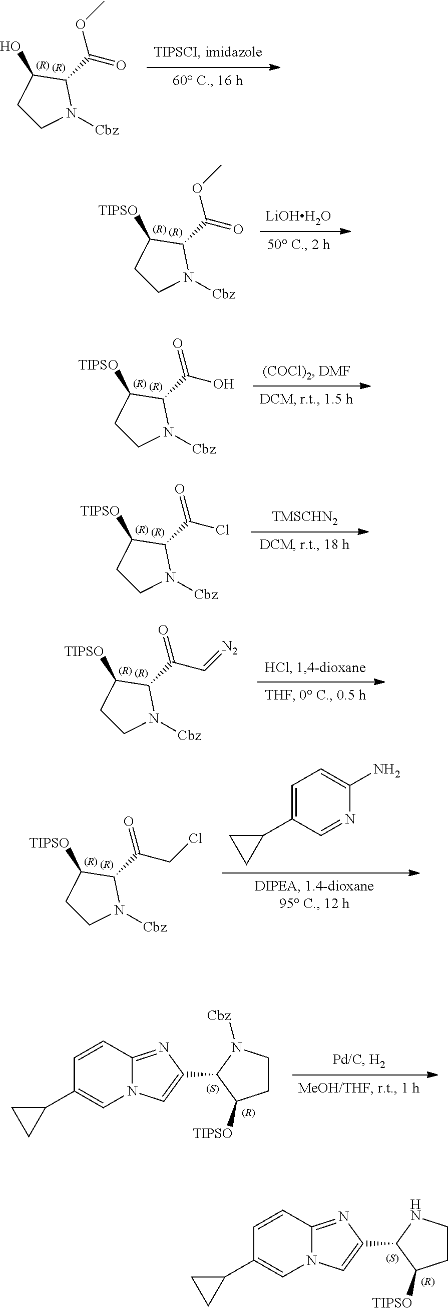

### Synthesis of 1-benzyl 2-methyl (2R,3R)-3-((triisopropylsilyl)oxy)pyrrolidine-1,2-dicarboxylate

To a mixture of 1-benzyl 2-methyl (2R,3R)-3-hydroxypyrrolidine-1,2-dicarboxylate (2.0 g, 7.2 mmol) in DCM (40 mL) was added imidazole (3.4 g, 50 mmol) and TIPSCl (6.9 g, 36 mmol) at 0° C. The resulting reaction mixture was stirred at 60° C. for 16 h under N₂ then cooled to room temperature. The reaction mixture was concentrated in vacuo in vacuo to give the crude. The residue was purified by column chromatography on silica gel (eluent: EtOAc/PE=0%-50%) to afford 1-benzyl 2-methyl (2R,3R)-3-((triisopropylsilyl)oxy) pyrrolidine-1,2-dicarboxylate (1.6 g, 50%) as yellow oil. ESI-MS [M+H]⁺: 436.2.

### Synthesis of (2R,3R)-1-((benzyloxy)carbonyl)-3-((triisopropylsilyl)oxy)pyrrolidine-2-carboxylic acid

To a mixture of 1-benzyl 2-methyl (2R,3R)-3-((triisopropylsilyl)oxy)pyrrolidine-1,2-dicarboxylate (1.6 g, 3.6 mmol) in MeOH (10 mL), THF (10 mL) and water (5 mL) was added LiOH·H₂O (1.1 g, 25 mmol) at room temperature. The resulting reaction mixture was stirred at 50° C. for 2 h under N₂. The reaction mixture was acidified (pH=4) with 1 N aq·HCl, diluted with H₂O (40 mL), then extracted with DCM (3×40 mL). The combined organics were concentrated in vacuo to afford (2R,3R)-1-((benzyloxy y)carbonyl)-3-((triisopropylsilyl)oxy)pyrrolidine-2-carboxylic acid (1.5 g, quant) as yellow oil. MS [M+H]+: 444.2.

### Synthesis of 6-cyclopropyl-2-((2S,3R)-3-((triisopropylsilyl)oxy)pyrrolidin-2-yl)imidazo[1,2-a]pyridine

Using a similar procedure to that for 2-((2R,4S)-4-((tert-butyldimethylsilyl)oxy)pyrrolidin-2-yl)-6-cyclopropylimidazo[1,2-a]pyridine, 6-cyclopropyl-2-((2S,3R)-3-((triisopropylsilyl)oxy)pyrrolidin-2-yl)imidazo[1,2-a]pyridine (65 mg) was synthesised from ((2R,3R)-1-((benzyloxy y)carbonyl)-3-((triisopropylsilyl)oxy)pyrrolidine-2-carboxylic acid. ESI-MS [M+H]⁺: 400.3

### Example 1

### Synthesis of (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-1)

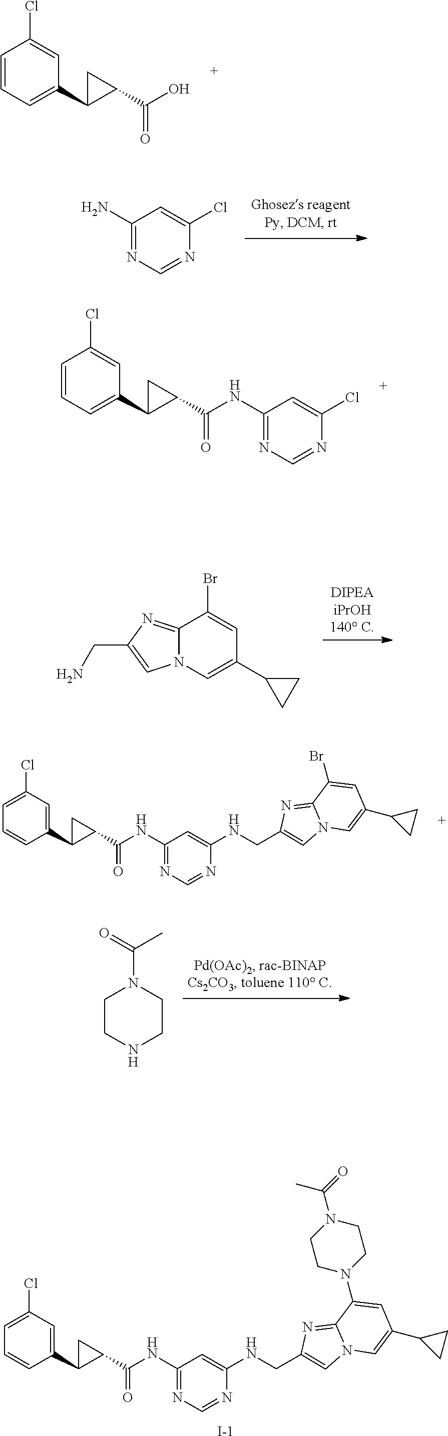

Synthesis of (1S,2S)-2-(3-chlorophenyl)-N-(6-chloropyrimidin-4-yl)cyclopropane-1-carboxamide. Ghosez's reagent (0.81 mL, 6.1 mmol) was added to a stirred solution of (1S,2S)-2-(3-chlorophenyl)cyclopropane-1-carboxylic acid (1.0 g, 5.1 mmol) in DCM (20 mL). The reaction was stirred at room temperature for 1.5 h, then 6-chloropyrimidin-4-amine (730 mg, 5.6 mmol) and pyridine (0.62 mL, 7.6 mmol) were added and the reaction was stirred at room temperature for 18 h. The reaction was diluted with DCM (60 mL) and NaHCO₃ (sat·aq·, 60 mL). The layers were separated and the aqueous layer was further extracted with DCM (2×30 mL). The combined organics were dried over MgSO4 then concentrated in vacuo. The residue was purified by column chromatography on silica gel, eluting with a gradient of 0-100% EtOAc in cyclohexane to give the title compound (1.1 g, 70%). ESI-MS (M+H)+: 308.1, ¹H NMR (400 MHz, CDCl₃) δ 8.63 (s, 1H), 8.24 (s, 2H), 7.24-7.21 (m, 2H), 7.09 (s, 1H), 7.04-7.00 (m, 1H), 2.66-2.60 (m, 1H), 1.82-1.77 (m, 2H), 1.51-1.45 (m, 1H).

Synthesis of (1S,2S)—N-(6-(((8-bromo-6-cyclopropylimidazo[1,2-a]pyridin-2-yl)methyl)amino)pyrimidin-4-yl)-2-(3-chlorophenyl)cyclopropane-1-carboxamide. (1S,2S)-2-(3-chlorophenyl)-N-(6-chloropyrimidin-4-yl)cyclopropane-1-carboxamide (1.0 g, 3.3 mmol), (8-bromo-6-cyclopropylimidazo[1,2-a]pyridin-2-yl)methanamine (950 mg, 3.6 mmol) and DIPEA (1.1 mL, 6.5 mmol) were suspended in iPrOH (20 mL). The reaction mixture was heated in a microwave at 140° C. for 4 h. The reaction mixture was concentrated in vacuo. The residue was purified by column chromatography on silica gel, eluting with a gradient of 0-20% (7 N NH₃ in MeOH) in DCM to give the title compound (1.3 g, 75%). ESI-MS (M+H)+: 537.3, 539.3, ¹H NMR (400 MHz, CDCl₃) δ 8.69-8.62 (m, 1H), 8.25 (s, 1H), 7.82 (s, 1H), 7.53 (s, 1H), 7.31 (s, 1H), 7.21-7.17 (m, 3H), 7.05 (d, J=2.0 Hz, 1H), 6.99-6.95 (m, 1H), 5.87-5.86 (m, 1H), 4.71-4.70 (m, 2H), 2.56 (ddd, J=4.0, 6.5, 9.2 Hz, 1H), 1.91-1.85 (m, 1H), 1.82-1.69 (m, 2H), 1.75 (s, 2H), 1.36 (ddd, J=4.7, 6.6, 8.2 Hz, 1H), 1.00-0.94 (m, 2H), 0.69-0.64 (m, 2H).

Synthesis of (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-1). (1S,2S)—N-(6-(((8-Bromo-6-cyclopropylimidazo[1,2-a]pyridin-2-yl)methyl)amino)pyrimidin-4-yl)-2-(3-chlorophenyl)cyclopropane-1-carboxamide (60 mg, 0.11 mmol), 1-acetylpiperazine (21 mg, 0.17 mmol), rac-BINAP (14 mg, 0.022 mmol), and Cs₂CO₃ (73 mg, 0.22 mmol) were suspended in toluene (4.0 mL) and degassed with nitrogen for 5 min. Pd(OAc)_{z}(2.5 mg, 0.011 mmol) was then added and the reaction was heated at 110° C. under a nitrogen atmosphere for 18 h. The reaction was cooled to room temperature, filtered through Celite®, and washed with DCM:MeOH (2:1, 10 mL). The filtrate was concentrated in vacuo. The residue was purified by reverse phase preparative HPLC to give the title compound (8.5 mg, 13%). ESI-MS (M+H)+: 585.3, ¹H NMR (400 MHz, DMSO) δ 10.59 (s, 1H), 8.19 (s, 1H), 7.90-7.89 (m, 1H), 7.84 (s, 1H), 7.53 (br s, 1H), 7.34-7.25 (m, 4H), 7.16-7.13 (m, 1H), 6.18 (d, J 1.2 Hz, 1H), 4.54 (br s, 2H), 3.62-3.60 (m, 4H), 3.51 (d, J=4.8 Hz, 2H), 3.41-3.36 (m, 2H), 2.42-2.31 (m, 2H), 2.05 (s, 3H), 1.88-1.81 (m, 1H), 1.49-1.37 (m, 2H), 0.89-0.84 (m, 2H), 0.68-0.63 (m, 2H).

Using a similar procedure to that used for (1S,2S)—N-(6-(((8-(4-acetylpiperazin-2-yl)-6-cyclopropylimidazo[1,2-a]pyridin-2-yl)methyl)amino)pyrimidin-4-yl)-2-(3-chlorophenyl)cyclopropane-1-carboxamide, the compounds in Table 1 were prepared from (1S,2S)—N-(6-(((8-bromo-6-cyclopropylimidazo[1,2-a]pyridin-2-yl)methyl)amino)pyrimidin-4-yl)-2-(3-chlorophenyl)cyclopropane-1-carboxamide and an appropriate coupling partner.

TABLE 1

| | | Coupling Partner/ |
|---|---|---|
| Example | Compound | Analytical Data |
| Example 2 | 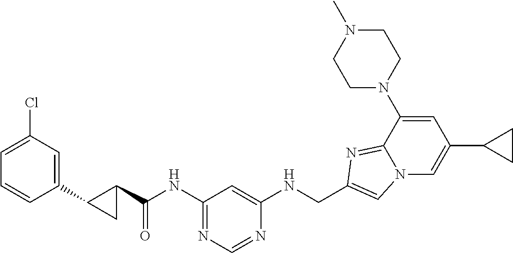 [SMILES: CN1CCN(c2cc(C3CC3)cn3cc(CNc4cc(NC(=O)[C@H]5C[C@@H]5c5cccc(Cl)c5)ncn4)nc23)CC1] | 1-methylpiperazine ESI-MS (M + H)+: 557.5, ¹H NMR (400 MHz, DMSO) δ 10.64 (1H, s), 8.24 (1H, s), 7.91 (2H, s), 7.56 (1H, s), 7.40 − 7.30 (4H, m), 7.20 (1H, d, J = 7.6 Hz), 6.19 (1H, s), 4.62 − 4.62 (2H, m), 3.52 (4H, s), 3.37 (4H, s), 2.48 − 2.38 (2H, m), 2.29 (3H, s), 1.94 − 1.86 (1H, m), 1.55 − 1.42 (2H, m), 0.94 − 0.88 (2H, m), 0.73 − 0.68 (2H, m); |
| | (1S,2S)-2-(3-chlorophenyl)-N-(6-(((6- | |
| | cyclopropyl-8-(4-methylpiperazin-1- | |
| | yl)imidazo[1,2-a]pyridin-2- | |
| | yl)methyl)amino)pyrimidin-4- | |
| | yl)cyclopropane-1-carboxamide (I-2) | |
| Example 3 | 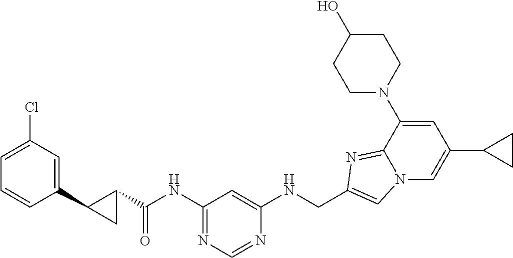 [SMILES: O=C(Nc1cc(NCc2cn3cc(C4CC4)cc(N4CCC(O)CC4)c3n2)ncn1)[C@@H]1C[C@H]1c1cccc(Cl)c1] | piperidin-4-ol ESI-MS (M + H)+: 558.5, ¹H NMR (400 MHz, DMSO) δ 10.68 (s, 1H), 8.24 (s, 1H), 8.01 (s, 2H), 7.71 (s, 1H), 7.36 − 7.30 (m, 2H), 7.28 (d, J = 6.3 Hz, 2H), 7.17 (d, J = 7.7 Hz, 1H), 4.65 − 4.65 (m, 3H), 3.73 − 3.66 (m, 2H), 2.96 (dd, J = 9.7, 9.7 Hz, 2H), 2.46 − 2.34 (m, 2H), 1.93 − 1.90 (m, 3H), 1.61 (d, J = 9.5 Hz, 2H), 1.52 − 1.39 (m, 2H), 0.92 (d, J = 6.5 Hz, 2H), 0.71 (d, J = 4.1 Hz, 2H). |
| | (1S,2S)-2-(3-chlorophenyl)-N-(6-(((6- | |
| | cyclopropyl-8-(4-hydroxypiperidin-1- | |
| | yl)imidazo[1,2-a]pyridin-2- | |
| | yl)methyl)amino)pyrimidin-4- | |
| | yl)cyclopropane-1-carboxamide (I-3) | |
| Example 4 | 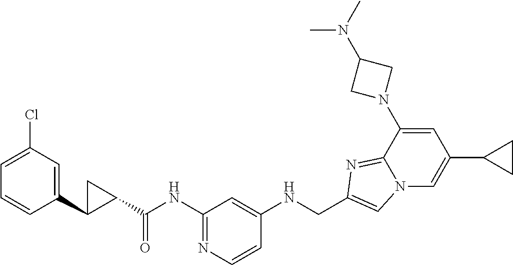 [SMILES: CN(C)C1CN(c2cc(C3CC3)cn3cc(CNc4ccnc(NC(=O)[C@H]5C[C@@H]5c5cccc(Cl)c5)c4)nc23)C1] | N,N-dimethylazetidin-3-amine ESI-MS (M + H)+: 556.5, ¹H NMR (400 MHz, DMSO) δ 10.38 − 10.37 (m, 1H), 7.77 (d, J = 5.9 Hz, 1H), 7.70 (s, 1H), 7.53 (s, 1H), 7.42 (s, 1H), 7.36 − 7.30 (m, 1H), 7.26 (d, J = 6.9 Hz, 2H), 7.18 − 7.12 (m, 2H), 6.35 (dd, J = 2.1, 5.8 Hz, 1H), 5.67 (s, 1H), 4.33 (d, J = 5.8 Hz, 2H), 4.24 (dd, J = 7.6, 7.6 Hz, 2H), 3.88 (dd, J = 6.3, 6.3 Hz, 2H), 2.42 − 2.33 (m, 2H), 2.18 (s, 6H), 1.86 − 1.78 (m, 1H), 1.49 − 1.33 (m, 2H), 0.89 − 0.83 (m, 2H), 0.67 − 0.61 (m, 2H). |
| | (1S,2S)-2-(3-chlorophenyl)-N-(4-(((6- | |
| | cyclopropyl-8-(3- | |
| | (dimethylamino)azetidin-1- | |
| | yl)imidazo[1,2-a]pyridin-2- | |
| | yl)methyl)amino)pyridin-2- | |
| | yl)cyclopropane-1-carboxamide (I-4) | |
| Example 5 | 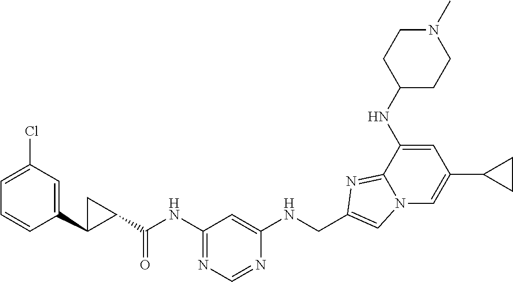 [SMILES: CN1CCC(Nc2cc(C3CC3)cn3cc(CNc4cc(NC(=O)[C@H]5C[C@@H]5c5cccc(Cl)c5)ncn4)nc23)CC1] | 1-methylpiperidin-4-amine ESI-MS (M + H)+: 571.6, ¹H NMR (400 MHz, DMSO) δ 10.64 (s, 1H), 8.35 (s, 1H), 8.24 (s, 1H), 7.84 (s, 1H), 7.59 (s, 1H), 7.53 (s, 1H), 7.40 − 7.29 (m, 4H), 7.22 − 7.19 (m, 1H), 6.68 (s, 1H), 5.36 (d, J = 8.3 Hz, 1H), 4.58 (s, 2H), 3.50 − 3.43 (m, 1H), 2.80 − 2.71 (m, 2H), 2.49 − 2.37 (m, 2H), 2.24 (s, 3H), 2.15 − 2.08 (m, 2H), 1.97 − 1.83 (m, 3H), 1.66 − 1.42 (m, 4H), 0.92 − 0.86 (m, 2H), 0.70 − 0.65 (m, 2H). |
| | (1S,2S)-2-(3-chlorophenyl)-N-(6-(((6- | |
| | cyclopropyl-8-((1-methylpiperidin-4- | |
| | yl)amino)imidazo[1,2-a]pyridin-2- | |
| | yl)methyl)amino)pyrimidin-4- | |
| | yl)cyclopropane-1-carboxamide, | |
| | formic acid salt (I-5) | |
| Example 6 | 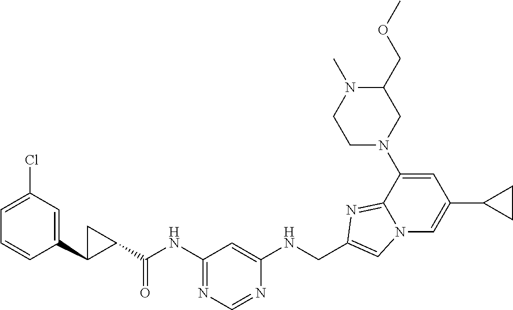 [SMILES: COCC1CN(c2cc(C3CC3)cn3cc(CNc4cc(NC(=O)[C@H]5C[C@@H]5c5cccc(Cl)c5)ncn4)nc23)CCN1C] | 2-(methoxymethyl)-1-methylpiperazine ESI-MS (M + H)+: 601.5, ¹H NMR (400 MHz, DMSO) δ 10.63 (s, 1H), 8.24 (s, 1H), 7.92 − 7.86 (m, 2H), 7.57 (s, 1H), 7.39 − 7.34 (m, 1H), 7.34 − 7.29 (m, 3H), 7.20 (d, J = 7.8 Hz, 1H), 6.20 (d, J = 1.0 Hz, 1H), 4.61 − 4.56 (m, 2H), 4.31 − 4.26 (m, 1H), 4.06 − 4.03 (m, 1H), 3.59 (dd, J = 4.2, 10.0 Hz, 1H), 3.44 − 3.39 (m, 1H), 3.33 (s, 3H), 2.91 − 2.84 (m, 2H), 2.73 − 2.64 (m, 1H), 2.46 − 2.38 (m, 4H), 2.33 (s, 3H), 1.95 − 1.87 (m, 1H), 1.55 − 1.42 (m, 2H), 0.94 − 0.89 (m, 2H), 0.72 − 0.67 (m, 2H). |
| | (1S,2S)-2-(3-chlorophenyl)-N-(6-(((6- | |
| | cyclopropyl-8-(3-(methoxymethyl)-4- | |
| | methylpiperazin-1-yl)imidazo[1,2- | |
| | a]pyridin-2- | |
| | yl)methyl)amino)pyrimidin-4- | |
| | yl)cyclopropane-1-carboxamide (I-6) | |
| Example 7 | 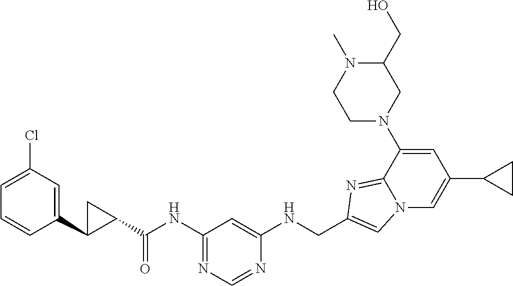 [SMILES: CN1CCN(c2cc(C3CC3)cn3cc(CNc4cc(NC(=O)[C@H]5C[C@@H]5c5cccc(Cl)c5)ncn4)nc23)CC1CO] | (1-methylpiperazin-2-yl)methanol ESI-MS (M + H)+: 587.4, ¹H NMR (400 MHz, DMSO) δ 10.61 (s, 1H), 8.35 (s, 1H), 8.21 (s, 1H), 7.91 − 7.80 (m, 2H), 7.53 (s, 1H), 7.36 − 7.25 (m, 4H), 7.16 (d, J = 7.8 Hz, 1H), 6.17 (s, 1H), 4.55 (s, 2H), 4.24 (s, 1H), 3.83 (d, J = 11.6 Hz, 1H), 3.66 (dd, J = 3.9, 11.2 Hz, 1H), 3.54 − 3.49 (m, 1H), 2.86 − 2.80 (m, 3H), 2.43 − 2.34 (m, 4H), 2.31 − 2.26 (m, 4H), 1.91 − 1.83 (m, 1H), 1.51 − 1.39 (m, 2H), 0.91 − 0.85 (m, 2H), 0.68 − 0.64 (m, 2H). |
| | (1S,2S)-2-(3-chlorophenyl)-N-(6-(((6- | |
| | cyclopropyl-8-(3-(hydroxymethyl)-4- | |
| | methylpiperazin-1-yl)imidazo[1,2- | |
| | a]pyridin-2- | |
| | yl)methyl)amino)pyrimidin-4- | |
| | yl)cyclopropane-1-carboxamide, | |
| | formic acid salt (I-7) | |
| Example 8 | 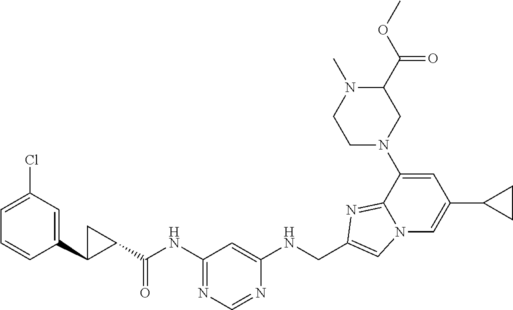 [SMILES: COC(=O)C1CN(c2cc(C3CC3)cn3cc(CNc4cc(NC(=O)[C@H]5C[C@@H]5c5cccc(Cl)c5)ncn4)nc23)CCN1C] | methyl 1-methylpiperazine-2- carboxylate ESI-MS (M + H)+: 615.4, ¹H NMR (400 MHz, DMSO) δ 10.64 (s, 1H), 8.25 (s, 1H), 7.93 (s, 1H), 7.89 (s, 1H), 7.57 (s, 1H), 7.40 − 7.34 (m, 1H), 7.32 − 7.29 (m, 3H), 7.20 (d, J = 7.8 Hz, 1H), 6.24 (d, J = 1.3 Hz, 1H), 4.58 (s, 2H), 3.99 − 3.96 (m, 1H), 3.82 (s, 1H), 3.72 (s, 3H), 3.27 − 3.20 (m, 1H), 3.14 − 3.09 (m, 1H), 2.48 − 2.38 (m, 4H), 2.34 (s, 3H), 1.94 − 1.87 (m, 1H), 1.55 − 1.42 (m, 2H), 0.92 (ddd, J = 4.1, 6.3, 8.4 Hz, 2H), 0.73 − 0.68 (m, 2H). |
| | methyl 4-(2-(((6-((15,2S)-2-(3- | |
| | chlorophenyl)cyclopropane-1- | |
| | carboxamido)pyrimidin-4- | |
| | yl)amino)methyl)-6- | |
| | cyclopropylimidazo[1,2-α]pyridin-8- | |
| | yl)-1-methylpiperazine-2-carboxylate | |
| | (I-8) | |
| Example 9 | 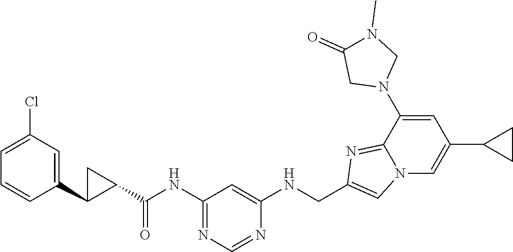 [SMILES: CN1CN(c2cc(C3CC3)cn3cc(CNc4cc(NC(=O)[C@H]5C[C@@H]5c5cccc(Cl)c5)ncn4)nc23)CC1=O] | 3-methylimidazolidin-4-one ESI-MS (M + H)+: 557.5, ¹H NMR (400 MHz, DMSO) δ 10.61 (s, 1H), 8.21 (s, 1H), 7.84 (s, 1H), 7.77 (s, 1H), 7.56 (s, 1H), 7.32 (d, J = 7.5 Hz, 2H), 7.27 (d, J = 5.4 Hz, 2H), 7.16 (d, J = 7.7 Hz, 1H), 5.82 (d, J = 1.3 Hz, 1H), 5.28 (s, 2H), 4.56 − 4.56 (m, 2H), 4.10 (s, 2H), 2.91 (s, 3H), 2.45 − 2.34 (m, 2H), 1.88 − 1.80 (m, 1H), 1.51 − 1.39 (m, 2H), 0.90 − 0.85 (m, 2H), 0.71 − 0.66 (m, 2H). |
| | (1S,2S)-2-(3-chlorophenyl)-N-(6-(((6- | |
| | cyclopropyl-8-(3-methyl-4- | |
| | oxoimidazolidin-1-yl)imidazo[1,2- | |
| | a]pyridin-2- | |
| | yl)methyl)amino)pyrimidin-4- | |
| | yl)cyclopropane-1-carboxamide (I-9) | |
| Example 10 | 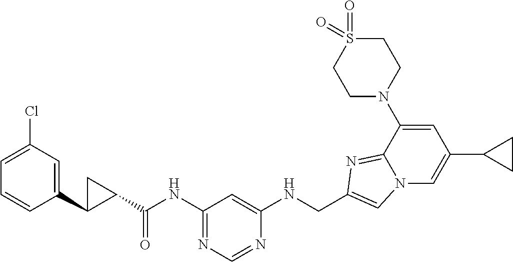 [SMILES: O=C(Nc1cc(NCc2cn3cc(C4CC4)cc(N4CCS(=O)(=O)CC4)c3n2)ncn1)[C@H]1C[C@@H]1c1cccc(Cl)c1] | thiomorpholine 1,1-dioxide ESI-MS (M + H)+: 592.4, ¹H NMR (400 MHz, DMSO) δ 10.62 (s, 1H), 8.20 (s, 1H), 7.93 (s, 1H), 7.86 (s, 1H), 7.57 (s, 1H), 7.36 − 7.26 (m, 4H), 7.16 (d, J = 7.7 Hz, 1H), 6.31 (s, 1H), 4.56 − 4.55 (m, 2H), 4.14 (d, J = 2.1 Hz, 4H), 3.25 (dd, J = 4.8, 4.8 Hz, 4H), 2.45 − 2.34 (m, 3H), 1.92 − 1.84 (m, 1H), 1.51 − 1.39 (m, 2H), 0.91 − 0.86 (m, 2H), 0.73 − 0.68 (m, 2H). |
| | (1S,2S)-2-(3-chlorophenyl)-N-(6-(((6- | |
| | cyclopropyl-8-(1,1- | |
| | dioxidothiomorpholino)imidazo[1,2- | |
| | a]pyridin-2- | |
| | yl)methyl)amino)pyrimidin-4- | |
| | yl)cyclopropane-1-carboxamide (I-10) | |
| Example 11 | 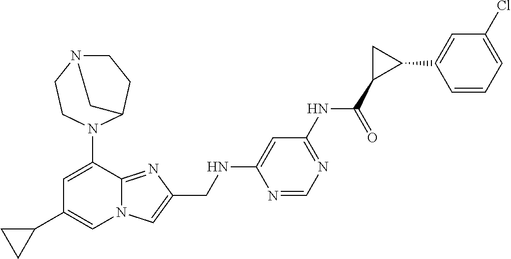 [SMILES: O=C(Nc1cc(NCc2cn3cc(C4CC4)cc(N4CCN5CCC4C5)c3n2)ncn1)[C@H]1C[C@@H]1c1cccc(Cl)c1] | 1,4-diazabicyclo[3.2.1]octane ESI-MS (M + H)+: 569.2, ¹H NMR (400 MHz, MeOD) δ 8.17 (s, 1H), 7.76 (s, 1H), 7.55 (s, 1H), 7.34 (d, J = 0.8 Hz, 1H), 7.28-7.24 (m, 1H), 7.20-7.18 (m, 2H), 7.10 (d, J = 7.6 Hz, 1H), 6.32 (s, 1H), 4.64 (s, 2H), 3.42 (d, J = 11.1 Hz, 1H), 3.26-3.20 (m, 4H), 3.08 − 3.01 (m, 2H), 2.89 (d, J = 8.9 Hz, 1H), 2.75 (d, J = 12.9 Hz, 1H), 2.49-2.44 (m, 1H), 2.20-2.18 (m, 1H), 1.90 − 1.81 (m, 3H), 1.62-1.58 (m, 2H), 0.93 − 0.90 (m, 2H), 0.70-0.67 (m, 2H). |
| | (1S,2S)-N-(6-(((8-(1,4- | |
| | diazabicyclo[3.2.1]octan-4-yl)-6- | |
| | cyclopropylimidazo[1,2-a]pyridin-2- | |
| | yl)methyl)amino)pyrimidin-4-yl)-2-(3- | |
| | chlorophenyl)cyclopropane-1- | |
| | carboxamide (I-11) | |
| Example 12 | 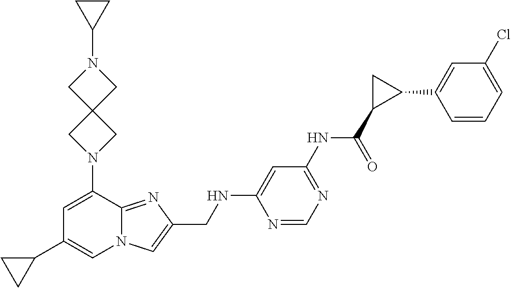 [SMILES: O=C(Nc1cc(NCc2cn3cc(C4CC4)cc(N4CC5(C4)CN(C4CC4)C5)c3n2)ncn1)[C@H]1C[C@@H]1c1cccc(Cl)c1] | 2-cyclopropyl-2,6- diazaspiro[3.3]heptane ESI-MS (M + H)+: 595.2, ¹H NMR (400 MHz, MeOD) δ 8.20 (s, 1H), 7.68 (s, 1H), 7.58 (s, 1H), 7.32 (s, 1H), 7.26 (t, J = 7.7 Hz, 1H), 7.21 − 7.17 (m, 2H), 7.10 (d, J = 7.6 Hz, 1H), 5.98 (s, 1H), 4.64 (s, 2H), 4.40 (s, 4H), 4.33 (s, 4H), 3.04 − 2.97 (m, 1H), 2.50 − 2.45 (m, 1H), 2.21 − 2.17 (m, 1H), 1.90 − 1.84 (m, 1H), 1.61 − 1.58 (m, 1H), 1.42 − 1.38 (m, 1H), 0.95 − 0.90 (m, 4H), 0.86 − 0.83 (m, 2H), 0.69 − 0.65 (m, 2H). |
| | (1S,2S)-2-(3-chlorophenyl)-N-(6-(((6- | |
| | cyclopropyl-8-(6-cyclopropyl-2,6- | |
| | diazaspiro[3.3]heptan-2- | |
| | yl)imidazo[1,2-a]pyridin-2- | |
| | yl)methyl)amino)pyrimidin-4- | |
| | yl)cyclopropane-1-scarboxamide (I-12) | |

### Examples 13-15

### Synthesis of (1S,2S)-2-(3-chlorophenyl)-N-(6-(((6-cyclopropyl-8-(3-(dimethylamino)pyrrolidin-1-yl)imidazo[1,2-a]pyridin-2-yl)methyl)amino)pyrimidin-4-yl)cyclopropane-1-carboxamide (I-13)

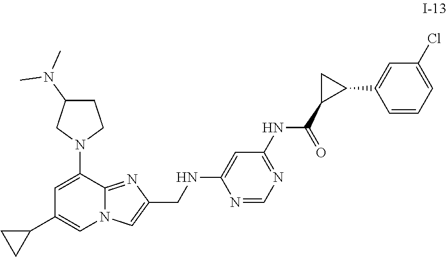 [SMILES: CN(C)C1CCN(c2cc(C3CC3)cn3cc(CNc4cc(NC(=O)[C@H]5C[C@@H]5c5cccc(Cl)c5)ncn4)nc23)C1]

The title compound was prepared in a similar manner to (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide as a mixture of diastereomers. ESI-MS [M+H]+: 571.2, ¹H NMR (400 MHz, DMSO) δ 10.58 (s, 1H), 8.17 (s, 1H), 7.79 (s, 1H), 7.61 (s, 1H), 7.47 (s, 1H), 7.29-7.24 (m, 4H), 7.15-7.13 (d, J=7.6 Hz, 1H), 5.70 (s, 1H), 4.52 (s, 2H), 3.93-3.84 (m, 2H), 3.67-3.62 (m, 1H), 3.50-3.48 (m, 1H), 2.78-2.72 (m, 1H), 2.42-2.34 (m, 2H), 2.19 (s, 6H), 1.83-1.74 (m, 2H), 1.50-1.38 (m, 3H), 0.85-0.81 (m, 2H), 0.64-0.60 (m, 2H).

The mixture was separated using chiral column separation [CHIRALPAK AD-H, 0.46 cm I.D×5 cm L MEOH=100, 1 mL/min, 35° C.) to give two diastereomers: (1S,2S)-2-(3-chlorophenyl)-N-(6-(((6-cyclopropyl-8-((R)-3-(dimethylamino)pyrrolidin-1-yl)imidazo[1,2-a]pyridin-2-yl)methyl)amino)pyrimidin-4-yl)cyclopropane-1-carboxamide and (1S,2S)-2-(3-chlorophenyl)-N-(6-(((6-cyclopropyl-8-((S)-3-(dimethylamino)pyrrolidin-1-yl)imidazo[1,2-a]pyridin-2-yl)methyl)amino)pyrimidin-4-yl)cyclopropane-1-carboxamide.

First eluting isomer (I-14): ESI-MS [M+H]+: 571.2. ¹H NMR (400 MHz, MeOD) δ 8.17 (s, 1H), 7.54 (s, 1H), 7.50 (s, 1H), 7.35 (s, 1H), 7.28-7.24 (m, 1H), 7.21-7.18 (m, 2H), 7.11-7.09 (m, 1H), 5.93 (s, 1H), 4.61 (s, 2H), 4.05-4.01 (m, 1H), 3.79-3.74 (m, 1H), 3.70-3.63 (m, 1H), 3.53-3.48 (m, 1H), 3.01-2.94 (m, 1H), 2.50-2.45 (m, 1H), 2.37 (s, 6H), 2.30-2.24 (m, 1H), 2.21-2.17 (m, 1H), 1.93-1.81 (m, 2H), 1.63-1.59 (m, 1H), 1.41-1.37 (m, 1H), 0.91-0.86 (m, 2H), 0.68-0.64 (m, 2H). RT=6.447 min, 99.82% e.e.

Second eluting isomer (I-15): ESI-MS [M+H]+: 571.2. ¹H NMR (400 MHz, MeOD) δ 8.16 (s, 1H), 7.56 (s, 1H), 7.49 (s, 1H), 7.34 (s, 1H), 7.27-7.23 (m, 1H), 7.19-7.17 (m, 2H), 7.10-7.08 (m, 1H), 5.95 (s, 1H), 4.60 (s, 2H), 3.98-3.93 (m, 1H), 3.80-3.75 (m, 1H), 3.68-3.59 (m, 2H), 3.18-3.11 (m, 1H), 2.47 (s, 6H), 2.33-2.26 (m, 1H), 2.20-2.16 (m, 1H), 1.98-1.93 (m, 1H), 1.86-1.79 (m, 1H), 1.62-1.57 (m, 1H), 1.39-1.35 (m, 1H), 0.89-0.86 (m, 2H), 0.67-0.63 (m, 2H). RT=8.348 min, 99.53% e.e.

### Example 14

### Synthesis of (1S,2S)-2-(3-chlorophenyl)-N-(6-(((6-cyclopropyl-8-(3-(methylamino)pyrrolidin-1-yl)imidazo[1,2-a]pyridin-2-yl)methyl)amino)pyrimidin-4-yl)cyclopropane-1-carboxamide (I-16)

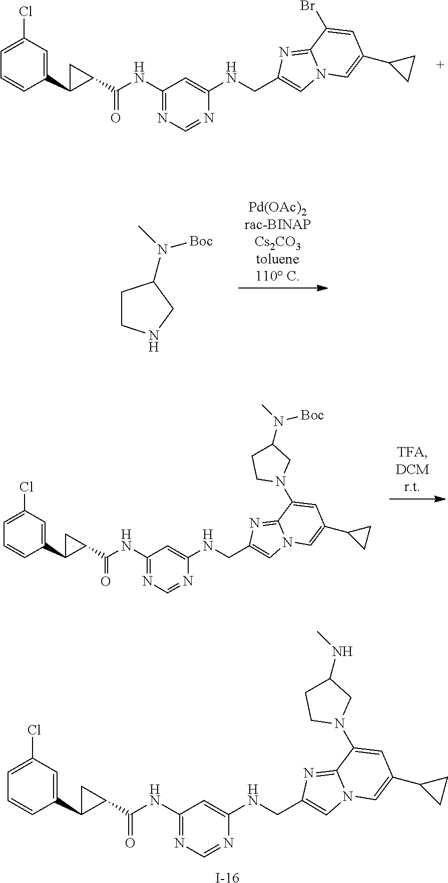

Synthesis of tert-butyl (1-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)pyrrolidin-3-yl)(methyl)carbamate. (1S,2S)—N-(6-(((8-Bromo-6-cyclopropylimidazo[1,2-a]pyridin-2-yl)methyl)amino)pyrimidin-4-yl)-2-(3-chlorophenyl)cyclopropane-1-carboxamide (150 mg, 0.28 mmol), tert-butyl methyl(pyrrolidin-3-yl)carbamate (84 mg, 0.42 mmol), rac-BINAP (35 mg, 0.056 mmol), and Cs₂CO₃ (180 mg, 0.56 mmol) were suspended in toluene (5.0 mL) and degassed with N₂ for 5 min. Pd(OAc)₂ (6.3 mg, 0.028 mmol) was then added and the reaction mixture was heated at 110° C. under a nitrogen atmosphere for 18 h. The reaction mixture was cooled to room temperature and filtered through Celite® and washed with DCM:MeOH (2:1, 10 mL). The reaction mixture was concentrated in vacuo. The residue was purified by column chromatography on silica gel, eluting with a gradient of 0-20% NH₃ in MeOH in DCM to give the title compound (120 mg, 66%). ESI-MS (M+H)+: 657.6, ¹H NMR (400 MHz, CDCl₃) δ 8.28 (s, 1H), 8.07 (s, 1H), 7.34-7.31 (m, 3H), 7.25-7.18 (m, 3H), 7.09 (s, 1H), 7.03-6.99 (m, 1H), 5.79 (s, 1H), 4.60 (s, 2H), 4.08-4.07 (m, 1H), 3.80 (dd, J=8.1, 10.6 Hz, 1H), 3.71-3.64 (m, 2H), 2.87 (s, 3H), 2.61-2.55 (m, 1H), 2.27-2.18 (m, 1H), 2.13-2.03 (m, 1H), 1.83-1.69 (m, 3H), 1.50-1.46 (m, 1OH), 1.43-1.37 (m, 1H), 0.91-0.86 (m, 2H), 0.66-0.61 (m, 2H).

Synthesis of (1S,2S)-2-(3-chlorophenyl)-N-(6-(((6-cyclopropyl-8-(3-(methylamino)pyrrolidin-1-yl)imidazo[1,2-a]pyridin-2-yl)methyl)amino)pyrimidin-4-yl)cyclopropane-1-carboxamide (I-16). Trifluoroacetic acid (0.28 mL, 3.7 mmol) was added to a stirred solution of tert-butyl (1-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)pyrrolidin-3-yl)(methyl)carbamate (120 mg, 0.22 mmol) in DCM (5.0 mL), the reaction was then stirred at room temperature for 3 h. The reaction was concentrated in vacuo. The residue was loaded on to an SCX cartridge in MeOH, washed with DCM:MeOH (3:1) and eluted with DCM:7N NH₃ in MeOH (3:1). The product was concentrated in vacuo and purified by reverse phase preparative HPLC to give the title compound (20.3 mg, 20%) as a formic acid salt.

ESI-MS (M+H)+: 557.4, ¹H NMR (400 MHz, DMSO) δ 10.60 (s, 1H), 8.20 (s, 1H), 7.81 (s, 1H), 7.63 (s, 1H), 7.49 (s, 1H), 7.36-7.26 (m, 4H), 7.16 (d, J=7.7 Hz, 1H), 5.70 (s, 1H), 4.54 (s, 2H), 3.91 (dd, J=6.3, 10.8 Hz, 1H), 3.78-3.71 (m, 1H), 3.68-3.62 (m, 1H), 3.59-3.53 (m, 1H), 2.44-2.34 (m, 6H), 2.13-2.05 (m, 1H), 1.85-1.77 (m, 2H), 1.51-1.39 (m, 2H), 0.88-0.82 (m, 2H), 0.66-0.61 (m, 2H).

### Example 15

### Synthesis of (1S,2S)-2-(3-chlorophenyl)-N-(6-(((6-cyclopropyl-8-(3-(dimethylamino)-2-oxopyrrolidin-1-yl)imidazo[1,2-a]pyridin-2-yl)methyl)amino)pyrimidin-4-yl)cyclopropane-1-carboxamide (I-17)

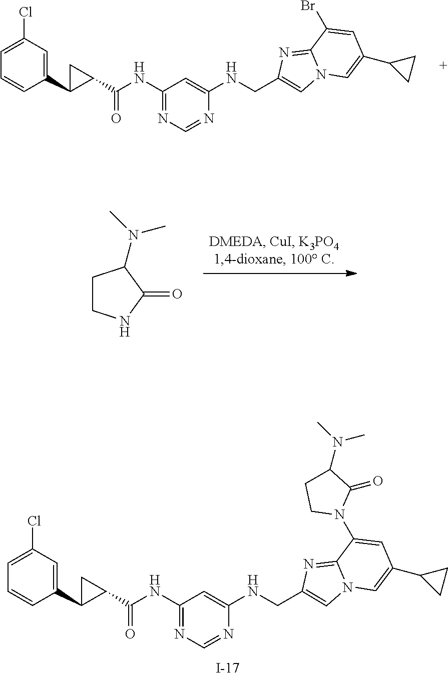

(1S,2S)—N-(6-(((8-Bromo-6-cyclopropylimidazo[1,2-a]pyridin-2-yl)methyl)amino)pyrimidin-4-yl)-2-(3-chlorophenyl)cyclopropane-1-carboxamide (100 mg, 0.19 mmol), 3-(dimethylamino)pyrrolidin-2-one (36 mg, 0.28 mmol), N,N-dimethylethylenediamine (12 μL, 0.11 mmol), and K₃PO₄ (79 mg, 0.37 mmol) were suspended in 1,4-dioxane (4.0 mL) and degassed with nitrogen for 5 min. CuI (11 mg, 0.059 mmol) was then added and the reaction was heated at 100° C. under a nitrogen atmosphere for 18 h. The reaction was then cooled to room temperature and filtered through Celite® and washed with DCM:MeOH (2:1, 10 mL). The reaction was concentrated in vacuo and the residue was purified by reverse phase preparative HPLC to give the title compound (23 mg, 21%). ESI-MS (M+H)+: 585.3, ¹H NMR (400 MHz, DMSO) δ 10.64 (s, 1H), 8.28 (d, J=1.1 Hz, 1H), 8.23 (s, 1H), 7.91 (s, 1H), 7.68 (s, 1H), 7.38-7.32 (m, 2H), 7.30-7.28 (m, 2H), 7.19 (d, J=6.9 Hz, 2H), 4.60 (s, 2H), 4.16 (dd, J=2.7, 9.2 Hz, 1H), 4.07-4.00 (m, 1H), 3.61 (dd, J=8.8, 8.8 Hz, 1H), 2.47-2.38 (m, 8H), 2.29-2.21 (m, 1H), 2.16-2.08 (m, 1H), 2.00-1.93 (m, 1H), 1.53-1.42 (m, 2H), 0.99-0.93 (m, 2H), 0.70-0.66 (m, 2H).

Using a similar procedure to that used for (1S,2S)-2-(3-chlorophenyl)-N-(6-(((6-cyclopropyl-8-(3-(dimethylamino)-2-oxopyrrolidin-1-yl)imidazo[1,2-a]pyridin-2-yl)methyl)amino)pyrimidin-4-yl)cyclopropane-1-carboxamide, the compound in Table 2 was prepared from (1S,2S)—N-(6-(((8-Bromo-6-cyclopropylimidazo[1,2-a]pyridin-2-yl)methyl)amino)pyrimidin-4-yl)-2-(3-chlorophenyl)cyclopropane-1-carboxamide and 4-(dimethylamino)pyrrolidin-2-one.

TABLE 2

| | | Coupling Partner |
|---|---|---|
| Example | Compound | Analytical Data |
| Example 18 | 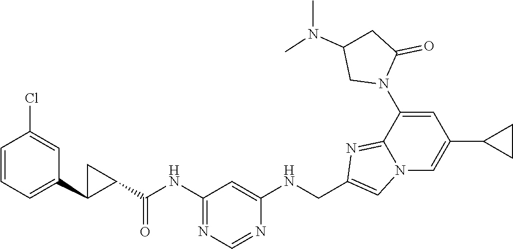 [SMILES: CN(C)C1CC(=O)N(c2cc(C3CC3)cn3cc(CNc4cc(NC(=O)[C@H]5C[C@@H]5c5cccc(Cl)c5)ncn4)nc23)C1] | 4-(dimethylamino)pyrrolidin-2-one ESI-MS (M + H)+: 585.3, ¹H NMR (400 MHz, DMSO) δ 10.63 (s, 1H), 8.28 (d, J = 1.3 Hz, 1H), 8.23 (s, 1H), 7.90 − 7.89 (m, 1H), 7.69 (s, 1H), 7.38 − 7.28 (m, 4H), 7.18 (d, J = 7.6 Hz, 1H), 7.15 (d, J = 1.5 Hz, 1H), 4.61 (s, 2H), 4.24 (dd, J = 7.5, 9.7 Hz, 1H), 4.04 (dd, J = 6.1, 9.9 Hz, 1H), 3.23 − 3.14 (m, 1H), 2.66 (dd, J = 8.5, 16.6 Hz, 1H), 2.51 − 2.39 (m, 3H), 2.22 (s, 6H), 1.99 − 1.91 (m, 1H), 1.54 − 1.41 (m, 2H), 0.95 (ddd, J = 4.4, 6.3, 8.4 Hz, 2H), 0.70 − 0.65 (m, 2H). |
| | (1S,2S)-2-(3-chlorophenyl)-N-(6-(((6- | |
| | cyclopropyl-8-(4-(dimethylamino)-2- | |
| | oxopyrrolidin-1-yl)imidazo[1,2- | |
| | a]pyridin-2- | |
| | yl)methyl)amino)pyrimidin-4- | |
| | yl)cyclopropane-1-carboxamide (I-18) | |

### Example 16

### Synthesis of (1S,2S)-2-(3-chlorophenyl)-N-(6-(((6-cyclopropyl-8-(5-oxoimidazolidin-1-yl)imidazo[1,2-a]pyridin-2-yl)methyl)amino)pyrimidin-4-yl)cyclopropane-1-carboxamide (I-19)

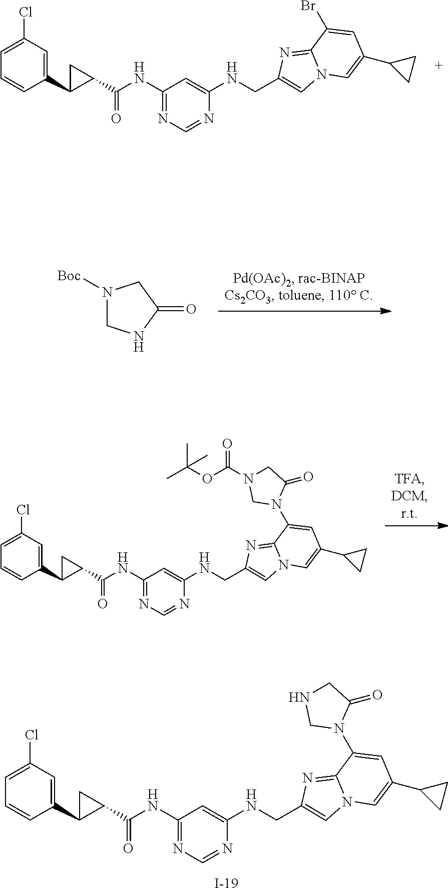

Synthesis of tert-butyl 3-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)-4-oxoimidazolidine-1-carboxylate. (1S,2S)—N-(6-(((8-Bromo-6-cyclopropylimidazo[1,2-a]pyridin-2-yl)methyl)amino)pyrimidin-4-yl)-2-(3-chlorophenyl)cyclopropane-1-carboxamide (80 mg, 0.15 mmol), tert-butyl 4-oxoimidazolidine-1-carboxylate (42 mg, 0.22 mmol), N,N-dimethylethylenediamine (3.2 μL, 0.030 mmol), and K₃PO₄ (63 mg, 0.30 mmol) were suspended in 1,4-dioxane (4.0 mL) and degassed with nitrogen for 5 min. CuI (2.8 mg, 0.015 mmol) was then added and the reaction was heated at 100° C. under a nitrogen atmosphere for 18 h. The reaction was cooled to room temperature and filtered through Celite® and washed with DCM:MeOH (2:1, 10 mL). The reaction was concentrated in vacuo. The residue was purified by column chromatography on silica gel, eluting with a gradient of 0-20% NH₃ in MeOH in DCM to give the title compound (50 mg, 52%). ESI-MS (M+H)+: 643.4

Synthesis of (1S,2S)-2-(3-chlorophenyl)-N-(6-(((6-cyclopropyl-8-(5-oxoimidazolidin-1-yl)imidazo[1,2-a]pyridin-2-yl)methyl)amino)pyrimidin-4-yl)cyclopropane-1-carboxamide (I-19). Trifluoroacetic acid (0.1 mL, 1.2 mmol) was added to a stirred solution of tert-butyl 3-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)-4-oxoimidazolidine-1-carboxylate (40 mg, 0.062 mmol) in DCM (2.0 mL). The reaction was stirred at room temperature for 3 h then concentrated in vacuo. The residue was loaded on to an SCX cartridge in MeOH, washed with DCM:MeOH (3:1), and eluted with DCM:NH₃ in MeOH (3:1). The product was concentrated in vacuo and purified by reverse phase preparative HPLC to give the title compound (8.1 mg, 24%). ESI-MS (M+H)+: 543.3, ¹H NMR (400 MHz, DMSO) δ 10.62 (s, 1H), 8.23-8.20 (m, 2H), 7.92-7.85 (m, 1H), 7.67 (s, 1H), 7.36-7.30 (m, 3H), 7.28-7.26 (m, 2H), 7.16 (d, J=7.7 Hz, 1H), 5.16 (s, 2H), 4.60-4.60 (m, 2H), 3.64 (s, 1H), 3.44 (s, 2H), 2.45-2.36 (m, 2H), 1.98-1.90 (m, 1H), 1.51-1.39 (m, 2H), 0.97-0.91 (m, 2H), 0.67-0.62 (m, 2H).

### Example 20

### Synthesis of (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-20)

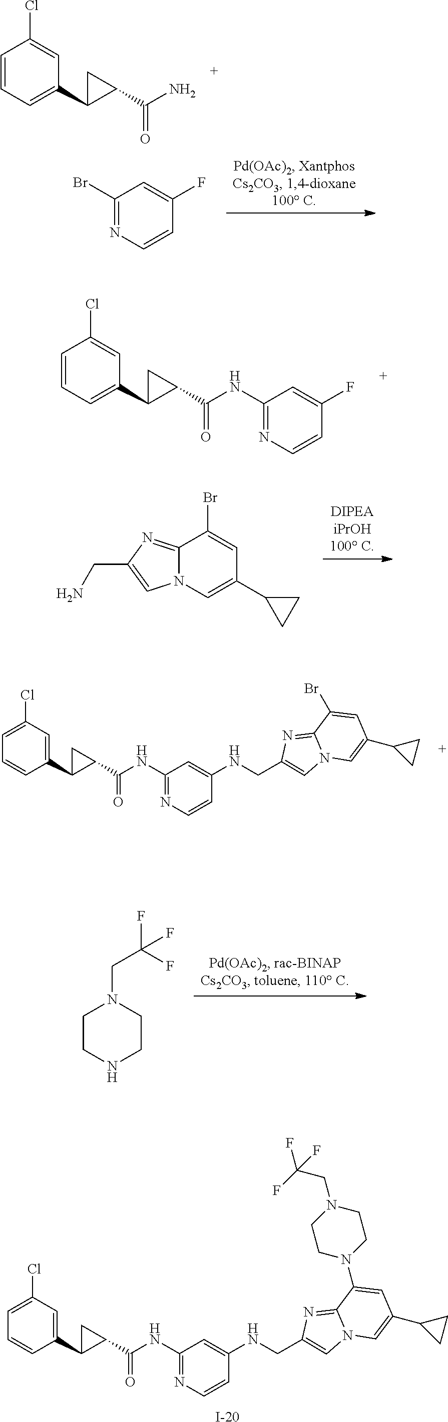

Synthesis of (1S,2S)-2-(3-chlorophenyl)-N-(4-fluoropyridin-2-yl)cyclopropane-1-carboxamide. 2-Bromo-4-fluoropyridine (500 mg, 2.8 mmol), (1S,2S)-2-(3-chlorophenyl)cyclopropane-1-carboxamide (611 mg, 3.13 mmol), Xantphos (330 mg, 0.57 mmol), and Cs₂CO₃ (1.9 g, 5.7 mmol) were suspended in 1,4-dioxane (20 mL) and degassed with nitrogen for 5 min. Pd(OAc)₂ (64 mg, 0.28 mmol) was then added and the reaction was heated at 110° C. under a nitrogen atmosphere for 4 h. The reaction was cooled to room temperature and filtered through Celite® and washed with DCM:MeOH (2:1, 30 mL). The reaction was concentrated in vacuo and the residue was purified by column chromatography on silica gel, eluting with a gradient of 0-60% EtOAc in cyclohexane to give the title compound (630 mg, 77%). ¹H NMR (400 MHz, CDCl₃) δ, 8.20-8.11 (m, 1H), 8.01 (dd, J=2.0, 11.1 Hz, 1H), 7.23-7.18 (m, 2H), 7.08 (dd, J=2.7, 2.7 Hz, 1H), 7.07-7.01 (m, 2H), 6.77-6.72 (m, 1H), 2.63-2.56 (m, 1H), 1.80-1.73 (m, 2H), 1.42-1.37 (m, 1H).

Synthesis of (1S,2S)—N-(4-(((8-bromo-6-cyclopropylimidazo[1,2-a]pyridin-2-yl)methyl)amino)pyridin-2-yl)-2-(3-chlorophenyl)cyclopropane-1-carboxamide. (1S,2S)-2-(3-Chlorophenyl)-N-(4-fluoropyridin-2-yl)cyclopropane-1-carboxamide (400 mg, 1.4 mmol), (8-bromo-6-cyclopropylimidazo[1,2-a]pyridin-2-yl)methanamine (370 mg, 1.4 mmol), and DIPEA (0.72 mL, 4.1 mmol) were suspended in iPrOH (20 mL). The reaction mixture was then heated at 100° C. in a sealed tube for 18 h. The reaction was concentrated in vacuo. The residue was purified by column chromatography on silica gel, eluting with a gradient of 0-20% NH₃ in MeOH in DCM to give the title compound (91 mg, 12%). ¹H NMR (400 MHz, CDCl₃) δ 9.68-9.68 (m, 1H), 7.82 (s, 1H), 7.67 (d, J=5.8 Hz, 1H), 7.62 (s, 1H), 7.55-7.52 (m, 1H), 7.21-7.15 (m, 3H), 7.02 (s, 1H), 7.01-6.95 (m, 1H), 6.18 (dd, J=2.3, 5.8 Hz, 1H), 5.20 (dd, J=5.7, 5.7 Hz, 1H), 4.54 (d, J=5.6 Hz, 2H), 2.56-2.50 (m, 1H), 2.28-2.09 (m, 2H), 2.05-1.98 (m, 1H), 1.90-1.83 (m, 2H), 1.75-1.66 (m, 1H), 1.32-1.26 (m, 1H), 1.00-0.94 (m, 2H), 0.69-0.64 (m, 2H).

Synthesis of (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide (I-20). (1S,2S)—N-(4-(((8-Bromo-6-cyclopropylimidazo[1,2-a]pyridin-2-yl)methyl)amino)pyridin-2-yl)-2-(3-chlorophenyl)cyclopropane-1-carboxamide (100 mg, 0.15 mmol), 1-(2,2,2-trifluoroethyl)piperazine (100 mg, 0.60 mmol), rac-BINAP (34 mg, 0.060 mmol), and Cs₂CO₃ (190 mg, 0.60 mmol) were suspended in toluene (11 mL) and degassed with nitrogen for 5 m. Pd(OAc)₂ (6.7 mg, 0.030 mmol) was then added and the reaction was heated at 100° C. under a nitrogen atmosphere for 76 h. The reaction was cooled to room temperature and filtered through Celite® and washed with DCM:MeOH (2:1, 10 mL). The reaction was concentrated in vacuo and the residue was purified by reverse phase preparative HPLC to give the title compound (12 mg, 13%) as a formic acid salt. ESI-MS (M+H)+: 624.5, ¹H NMR (400 MHz, DMSO) δ 10.35 (s, 1H), 7.93 (s, 1H), 7.82-7.79 (m, 1H), 7.59 (s, 1H), 7.50 (s, 1H), 7.36-7.28 (m, 3H), 7.21-7.11 (m, 2H), 6.38 (dd, J=2.0, 5.8 Hz, 1H), 6.22 (d, J=1.3 Hz, 1H), 4.39 (d, J=5.8 Hz, 2H), 3.57-3.51 (m, 4H), 3.30 (q, J=10.2 Hz, 2H), 2.90-2.84 (m, 4H), 2.45-2.38 (m, 2H), 1.96-1.86 (m, 1H), 1.53-1.47 (m, 1H), 1.42-1.36 (m, 1H), 0.95-0.89 (m, 2H), 0.74-0.69 (n, 2H).

Using a similar procedure to that used for (1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide, the compounds in Table 3 were prepared from (1S,2S)—N-(4-(((8-Bromo-6-cyclopropylimidazo[1,2-a]pyridin-2-yl)methyl)amino)pyridin-2-yl)-2-(3-chlorophenyl)cyclopropane-1-carboxamide and an appropriate coupling partner.

TABLE 3

| | | Coupling Partner/ |
|---|---|---|
| Example | Compound | Analytical Data |
| Example 21 | 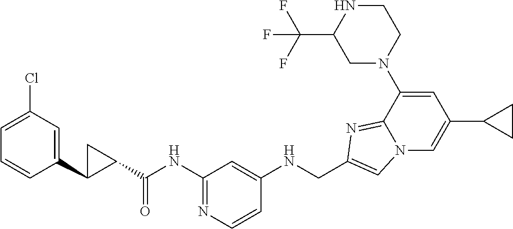 [SMILES: O=C(Nc1cc(NCc2cn3cc(C4CC4)cc(N4CCNC(C(F)(F)F)C4)c3n2)ccn1)[C@H]1C[C@@H]1c1cccc(Cl)c1] | 2-(trifluoromethyl)piperazine ESI-MS (M + H)+: 610.5, ¹H NMR (400 MHz, DMSO) δ 10.35 (s, 1H), 8.57 (s, 1H), 7.95 (s, 1H), 7.82 − 7.79 (m, 1H), 7.60 (s, 1H), 7.49 (s, 1H), 7.39 − 7.28 (m, 3H), 7.21 − 7.11 (m, 2H), 6.39 (dd, J = 2.3, 5.8 Hz, 1H), 6.30 (d, J = 1.3 Hz, 1H), 4.45 − 4.37 (m, 3H), 4.08 (d, J = 10.6 Hz, 1H), 3.62 (t, J = 9.5 Hz, 1H), 3.12 − 3.07 (m, 1H), 3.00 − 2.96 (m, 1H), 2.87 − 2.71 (m, 2H), 2.44 − 2.38 (m, 2H), 1.96 − 1.88 (m, 1H), 1.54 − 1.36 (m, 2H), 0.95 − 0.89 (m, 2H), 0.75 − 0.69 (m, 2H). |
| | (1S,2S)-2-(3-chlorophenyl)-N-(4-(((6- | |
| | cyclopropyl-8-(3- | |
| | (trifluoromethyl)piperazin-1- | |
| | yl)imidazo[1,2-a]pyridin-2- | |
| | yl)methyl)amino)pyridin-2- | |
| | yl)cyclopropane-1-carboxamide (I-21) | |
| Example 22 | 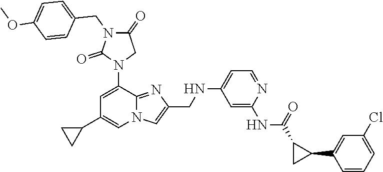 [SMILES: COc1ccc(CN2C(=O)CN(c3cc(C4CC4)cn4cc(CNc5ccnc(NC(=O)[C@H]6C[C@@H]6c6cccc(Cl)c6)c5)nc34)C2=O)cc1] | 3-(4-methoxybenzyl)imidazolidine-2,4-dione ESI-MS [M + H]+: 676.2; ¹H NMR (400 MHz, DMSO) δ 10.34 (s, 1H), 8.26 (s, 1H), 7.76 − 7.72 (m, 2H), 7.35 − 7.24 (m, 7H), 7.14 (d, J = 7.3 Hz, 1H), 6.91 (d, J = 8.5 Hz, 2H), 6.35 (s, 1H), 4.99 (s, 2H), 4.61 (s, 2H), 4.37 (s, 2H), 3.73 (s, 3H), 2.40 − 2.31 (m, 2H), 1.96 − 1.92 (m, 1H), 1.47 − 1.43 (m, 1H), 1.38 − 1.34 (m, 1H), 0.95 − 0.92 (m, 2H), 0.66 − 0.62 (m, 2H). 1,4-diazabicyclo[3.2.1]octane |
| | (1S,2S)-2-(3-chlorophenyl)-N-(4-(((6- | |
| | cyclopropyl-8-(3-(4-methoxybenzyl)- | |
| | 2,4-dioxoimidazolidin-1- | |
| | yl)imidazo[1,2-a]pyridin-2- | |
| | yl)methyl)amino)pyridin-2- | |
| | yl)cyclopropane-1-carboxamide (I-22) | |
| Example 23 | 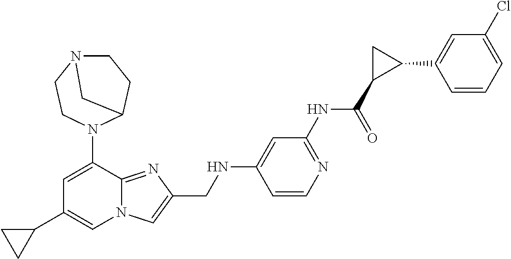 [SMILES: O=C(Nc1cc(NCc2cn3cc(C4CC4)cc(N4CCN5CCC4C5)c3n2)ccn1)[C@H]1C[C@@H]1c1cccc(Cl)c1] | ESI-MS [M + H]+: 568.2.. ¹H NMR (400 MHz, MeOD) δ 7.76 (d, J = 3.4 Hz, 2H), 7.56 (s, 1H), 7.37 (s, 1H), 7.27-7.24 (m, 1H), 7.19-7.17 (m, 2H), 7.09 (d, J = 7.6 Hz, 1H), 6.41-6.39 (m, 1H), 6.33 (s, 1H), 4.48 (s, 2H), 3.44 (d, J = 11.2 Hz, 1H), 3.25-3.22 (m, 4H), 3.13-3.02 (m, 2H), 2.92 (d, J = 9.0 Hz, 1H), 2.79 (d, J = 10.5 Hz, 1H), 2.48-2.43 (m, 1H), 2.19-2.15 (m, 1H), 1.91 − 1.85 (m, 3H), 1.61-1.57 (m, 2H), 0.93 − 0.91 (m, 2H), 0.70-0.67 m, 2H). |
| | (1S,2S)-N-(4-(((8-(1,4- | |
| | diazabicyclo[3.2.1]octan-4-yl)-6- | |
| | cyclopropylimidazo[1,2-a]pyridin-2- | |
| | yl)methyl)amino)pyridin-2-yl)-2-(3- | |
| | chlorophenyl)cyclopropane-1- | |
| | carboxamide (I-23) | |
| Example 24 | 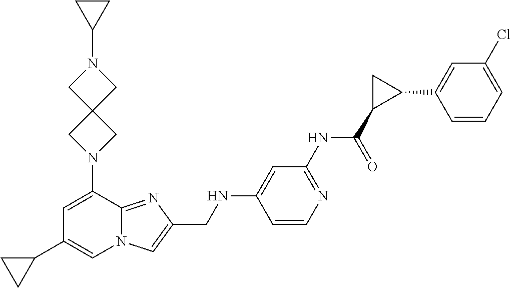 [SMILES: O=C(Nc1cc(NCc2cn3cc(C4CC4)cc(N4CC5(C4)CN(C4CC4)C5)c3n2)ccn1)[C@H]1C[C@@H]1c1cccc(Cl)c1] | 2-cyclopropyl-2,6- diazaspiro[3.3]heptane ESI-MS [M + H] +: 594.2. ¹H NMR (400 MHz, DMSO) δ 10.40 (s, 1H), 7.73 (d, J = 5.9 Hz, 1H), 7.65 (s, 1H), 7.48 (s, 1H), 7.32 − 7.21 (m 2H), 7.18 − 7.10 (m, 2H), 7.18 − 7.10 (m, 2H), 6.32 (d, J = 4.8 Hz, 1H), 5.63 (s, 1H), 4.28 (d, J = 5.6 Hz, 2H), 4.17 (s, 4H), 3.68 (s, 4H), 2.38 − 2.30 (m, 3H), 1.81 − 1.74 (m, 1H), 1.45 − 1.40 (m, 1H), 1.36 − 1.31 (m, 1H), 0.83 − 0.79 (m, 2H), 0.65 − 0.56 (m, 2H), 0.52 − 0.26 (m, 4H). |
| | (1S,2S)-2-(3-chlorophenyl)-N-(4-(((6- | |
| | cyclopropyl-8-(6-cyclopropyl-2,6- | |
| | diazaspiro[3.3]heptan-2- | |
| | yl)imidazo[1,2-a]pyridin-2- | |
| | yl)methyl)amino)pyridin-2- | |
| | yl)cyclopropane-1-carboxamide (I-24) | |
| Example 25 | 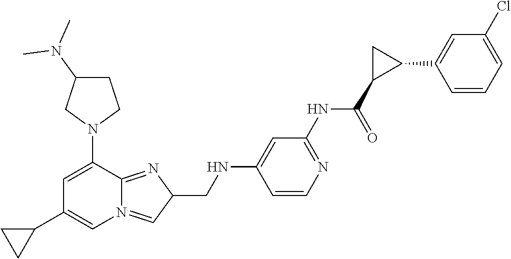 | 3-(dimethylamino)pyrrolidine ESI-MS [M + H] +: 570.2. ¹H NMR (400 MHz, DMSO) 8 10.30 (s, 1H), 7.74 (d, J = 5.8 Hz, 1H), 7.62 (s, 1H), 7.50 (s, 1H), 7.44 (s, 1H), 7.33 − 7.23 (m, 3H), 7.14 (d, J = 7.6 Hz, 1H), 7.05 (t, J = 5.5 Hz, 1H), 6.33 (dd, J = 5.8, 2.1 Hz, 1H), 5.71 (s, 1H), 4.31 (d, J = 5.3 Hz, 2H), 3.94 − 3.86 (m, 2H), 3.69 − 3.62 (m, 1H), 3.40 − 3.35 (m, 1H), 2.79 − 2.71 (m, 1H), 2.39 − 2.32 (m, 2H), 2.20 (s, 6H), 2.14 − 2.08 (m, 1H), 1.83 − 1.73 (m, 2H), 1.46 − 1.42 (m, 1H), 1.36 − 1.31 (m, 1H), 0.87 − 0.81 (m, 2H), 0.65 − 0.61 (m, 2H). |
| | (1S,2S)-2-(3-chlorophenyl)-N-(4-(((6- | |
| | cyclopropyl-8-(3- | |
| | (dimethylamino)pyrrolidin-1- | |
| | yl)imidazo[1,2-a]pyridin-2- | |
| | yl)methyl)amino)pyridin-2- | |
| | yl)cyclopropane-1-carboxamide (I-25) | |
| Example 26 | 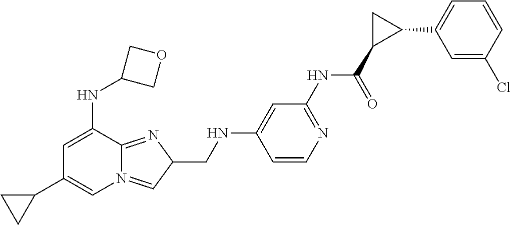 | 3-amino-oxetane ESI-MS [M + H]+: 529.2, ¹H NMR (400 MHz, DMSO) δ 10.35 (s, 1H), 7.76 (d, J = 5.8 Hz, 1H), 7.62 (s, 1H), 7.52 (s, 1H), 7.42 (s, 1H), 7.33 − 7.29 (m, 1H), 7.25 (d, J = 6.7 Hz, 2H), 7.14 (d, J = 7.6 Hz, 1H), 7.09 (s, 1H), 6.57 (d, J = 6.2 Hz, 1H), 6.34 − 6.29 (m, 1H), 5.63 (s, 1H), 4.84 (t, J = 6.4 Hz, 2H), 4.75 − 4.68 (m, 1H), 4.59 (t, J = 5.9 Hz, 2H), 4.35 (d, J = 4.8 Hz, 2H), 2.40 − 2.33 (m, 2H), 1.82 − 1.76 (m, 1H), 1.47 − 1.42 (m, 1H), 1.37 − 1.33 (m, 1H), 0.86 − 0.81 (m, 2H), 0.63 − 0.59 (m, 2H). |
| | (1S,2S)-2-(3-chlorophenyl)-N-(4-(((6- | |
| | cyclopropyl-8-(oxetan-3- | |
| | ylamino)imidazo[1,2-a]pyridin-2- | |
| | yl)methyl)amino)pyridin-2- | |
| | yl)cyclopropane-1-carboxamide (I-26) | |

### Example 17

### Synthesis of (1S,2S)—N-(6-(((R)-1-(6-cyclopropyl-8-(7-oxo-2-oxa-6-azaspiro[3.4]octan-6-yl)imidazo[1,2-a]pyridin-2-yl)ethyl)amino)-2-methylpyrimidin-4-yl)-2-(4-methylpyrimidin-2-yl)cyclopropane-1-carboxamide (I-27)

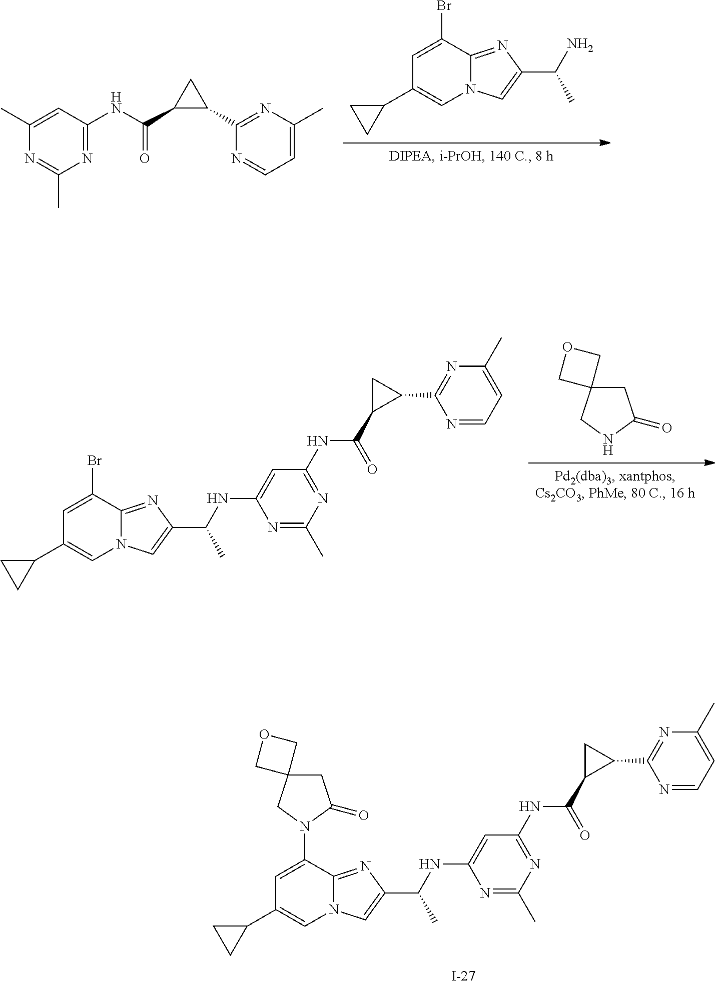

Synthesis of (1S,2S)—N-(6-(((R)-1-(8-bromo-6-cyclopropylimidazo[1,2-a]pyridin-2-yl)ethyl)amino)-2-methylpyrimidin-4-yl)-2-(4-methylpyrimidin-2-yl)cyclopropane-1-carboxamide. A mixture of (1S,2S)—N-(6-chloro-2-methylpyrimidin-4-yl)-2-(4-methylpyrimidin-2-yl)cyclopropane-1-carboxamide (200 mg, 0.66 mmol), (R)-1-(8-bromo-6-cyclopropylimidazo[1,2-a]pyridin-2-yl)ethan-1-amine (276 mg, 0.99 mmol) and DIPEA (255 mg, 1.97 mmol) in i-PrOH (2 mL) was stirred in a sealed tube. After degassing with N₂ for 1 min, the reaction mixture was irradiated in microwave at 140° C. for 8 h. The reaction was cooled to room temperature and concentrated in vacuo to give the crude, which was purified by preparative TLC (eluent: DCM/MeOH=10/1) to give (1S,2S)—N-(6-(((R)-1-(8-bromo-6-cyclopropylimidazo[1,2-a]pyridin-2-yl)ethyl)amino)-2-methylpyrimidin-4-yl)-2-(4-methylpyrimidin-2-yl)cyclopropane-1-carboxamide (200 mg, 55.4%) as a yellow solid. ESI-MS [M+H]+: 547.2.

Synthesis of (1S,2S)—N-(6-(((R)-1-(6-cyclopropyl-8-(7-oxo-2-oxa-6-azaspiro[3.4]octan-6-yl)imidazo[1,2-a]pyridin-2-yl)ethyl)amino)-2-methylpyrimidin-4-yl)-2-(4-methylpyrimidin-2-yl)cyclopropane-1-carboxamide (I-27). To a mixture of (1S,2S)—N-(6-(((R)-1-(8-bromo-6-cyclopropylimidazo[1,2-a]pyridin-2-yl)ethyl)amino)-2-methylpyrimidin-4-yl)-2-(4-methylpyrimidin-2-yl)cyclopropane-1-carboxamide (100 mg, 0.18 mmol), 2-oxa-6-azaspiro[3.4]octan-7-one (46 mg, 0.36 mmol), and Cs₂CO₃ (117 mg, 0.36 mmol) in toluene (5 mL) was added Pd₂(dba)₃ (33 mg, 0.036 mmol) and Xantphos (21 mg, 0.036 mmol). The reaction mixture was stirred at 80° C. for 16 h under N₂. After cooling to room temperature, water (20 mL) was added and the mixture was extracted with EtOAc (3×20 mL). The combined organic layers were washed with brine (30 mL), dried over anhydrous Na₂SO₄, and concentrated in vacuo to give the crude, which was purified by preparative TLC (eluent: DCM/MeOH=10/1) to give (1S,2S)—N-(6-(((R)-1-(6-cyclopropyl-8-(7-oxo-2-oxa-6-azaspiro[3.4]octan-6-yl)imidazo[1,2-a]pyridin-2-yl)ethyl)amino)-2-methylpyrimidin-4-yl)-2-(4-methylpyrimidin-2-yl)cyclopropane-1-carboxamide (33 mg, 30.9%) as a white solid. ESI-MS [M+H]⁺: 594.3. ¹H NMR (400 MHz, DMSO) δ 10.63 (s, 1H), 8.49 (d, J=5.1 Hz, 1H), 8.21 (s, 1H), 7.60 (s, 2H), 7.18 (d, J=5.1 Hz, 1H), 7.10 (s, 2H), 5.40 (s, 1H), 4.63-4.62 (m, 4H), 4.44-4.43 (m, 2H), 2.87 (s, 2H), 2.59-2.51 (m, 2H), 2.39 (s, 3H), 2.22 (s, 3H), 1.93-1.83 (m, 1H), 1.47-1.45 (m, 5H), 0.89-0.87 (m, 2H), 0.60-0.59 (m, 2H).

### Synthesis of 3-tritylimidazolidine-2,4-dione

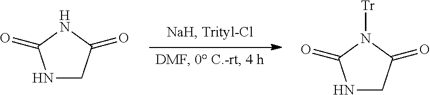

To a mixture of imidazolidine-2,4-dione (100 mg, 1.0 mmol) in DMF (4 mL) was added NaH (60 mg, 60% dispersion in mineral oil, 1.50 mmol) under N₂. The resulting reaction was stirred at rt for 0.5 h, then trityl chloride (418 mg, 1.50 mmol) in DMF (1 mL) was added. The reaction mixture was stirred at rt for 4 h. The reaction was quenched with water (20 mL) and extracted with EtOAc (30 mL×3). The combined organic layers were washed with brine (10 mL), dried over anhydrous Na₂SO₄, and concentrated in vacuo to give the crude product, which was purified by column chromatography (eluent: EtOAc/PE=1/100˜100/1) to give 3-tritylimidazolidine-2,4-dione (150 mg, 44%) as a pale solid. ESI-MS [M+H]+: 343.2.

### Synthesis of 3-(2-(benzyloxy)ethyl)imidazolidine-2,4-dione

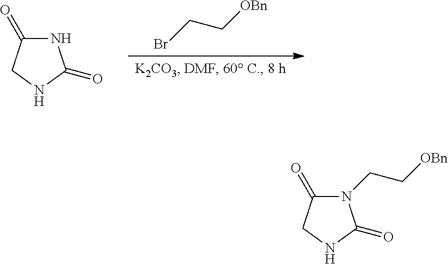

A mixture of imidazolidine-2,4-dione (1.0 g, 10 mmol), ((2-bromoethoxy)methyl)benzene (2.14 g, 10 mmol), and K₂CO₃ (2.76 g, 20 mmol) in DMF (30 mL) was stirred at 60° C. for 8 h. The reaction was quenched with water (100 mL) and extracted with EtOAc (50 mL×3). The combined organic layers were washed with water (50 mL), dried over anhydrous Na₂SO₄, and concentrated in vacuo to give the crude product, which was purified by column chromatography (eluent: PE/EtOAc=10/1) to give 3-(2-(benzyloxy)ethyl)imidazolidine-2,4-dione (1.2 g, 51%) as a white solid. ESI-MS [M+H]⁺: 235.2.

### Synthesis of 8-bromo-2-(((6-chloropyrimidin-4-yl)oxy)methyl)-6-cyclopropylimidazo[1,2-a]pyridine

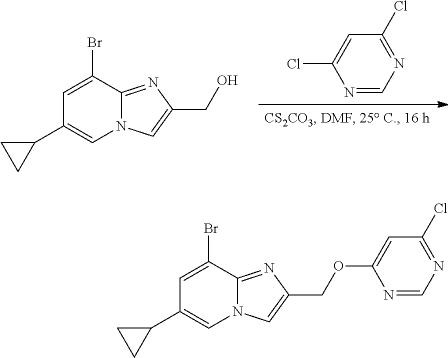

A mixture of (8-bromo-6-cyclopropylimidazo[1,2-a]pyridin-2-yl)methanol (390 mg, 1.46 mmol), 4,6-dichloropyrimidine (434 mg, 2.91 mmol), and Cs₂CO₃ (1.4 g, 4.30 mmol) in DMF (6 ml) was stirred at 25° C. for 16 h. Water (30 mL) was added and the reaction mixture was extracted with EtOAc (3×50 mL). The combined organic layers were washed with brine (30 mL), dried over anhydrous Na₂SO₄, and concentrated in vacuo to give the crude, which was purified by column chromatography (eluent: PE/EtOAc=2/1) to give 8-bromo-2-(((6-chloropyrimidin-4-yl)oxy)methyl)-6-cyclopropylimidazo[1,2-a]pyridine (420 mg, 75.8%) as a yellow solid. ESI-MS [M+H]⁺: 378.9

### Synthesis of 1-(2-(((6-chloropyrimidin-4-yl)oxy)methyl)-6-cyclopropylimidazo[1,2-a]pyridin-8-yl)-3-methylimidazolidine-2,4-dione

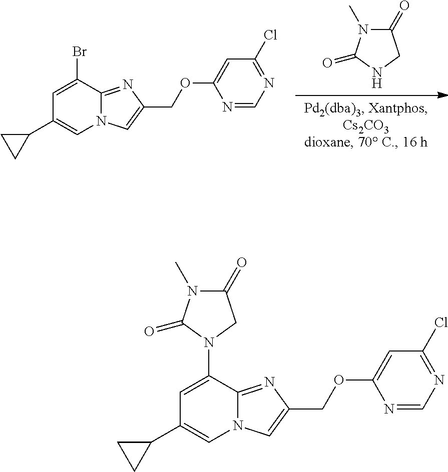

To a mixture of 8-bromo-2-(((6-chloropyrimidin-4-yl)oxy)methyl)-6-cyclopropylimidazo[1,2-a]pyridine (200 mg, 0.53 mmol), 3-methylimidazolidine-2,4-dione (48.2 mg, 0.42 mmol), and Cs₂CO₃ (431 mg, 1.32 mmol) in dioxane (6 mL) was added Pd₂(dba)₃ (73 mg, 0.080 mmol) and XantPhos (92 mg, 0.159 mmol). The reaction mixture was stirred at 70° C. for 16 h under N₂. The reaction mixture was cooled to room temperature, filtered through Celite®, and the filter cake was washed with DCM/MeOH (10/1, 50 mL). The filtrate was concentrated in vacuo to give the crude, which was purified by column chromatography (eluent: DCM/MeOH=50/1˜20/1) to give 1-(2-(((6-chloropyrimidin-4-yl)oxy)methyl)-6-cyclopropylimidazo[1,2-a]pyridin-8-yl)-3-methylimidazolidine-2,4-dione (60 mg, 34.6%) as a yellow solid. ESI-MS [M+H]+: 413.1.

The compounds in Table 4 were prepared in a similar manner to 1-(2-(((6-chloropyrimidin-4-yl)oxy)methyl)-6-cyclopropylimidazo[1,2-a]pyridin-8-yl)-3-methylimidazolidine-2,4-dione from 8-bromo-2-(((6-chloropyrimidin-4-yl)oxy)methyl)-6-cyclopropylimidazo[1,2-a]pyridine and an appropriate coupling partner.

TABLE 4

| | Coupling partner/ |
|---|---|
| Compound | Analytical Data |
| 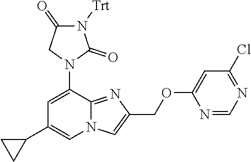 | 3- tritylimidazolidine- 2,4- dione ESI-MS [M + H]⁺: 641.2 |
| 1-(2-(((6-chloropyrimidin-4-yl)oxy)methyl)- | |
| 6-cyclopropylimidazo[1,2-a]pyridin- | |
| 8-yl)-3-tritylimidazolidine-2,4-dione | |
| 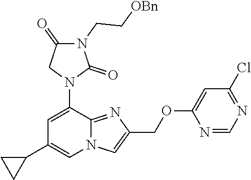 [SMILES: O=C1CN(c2cc(C3CC3)cn3cc(COc4cc(Cl)ncn4)nc23)C(=O)N1CCOCc1ccccc1] | 3-(2-(benzyloxy) ethyl) imidazolidine- 2,4-dione ESI-MS [M + H]⁺: 533.2 |
| 3-(2-(benzyloxy)ethyl)-1-(2-(((6-chloropyrimidin- | |
| 4-yl)oxy)methyl)-6-cyclopropylimidazo | |
| [1,2-a]pyridin-8-yl)imidazolidine-2,4-dione | |

### Examples 28-30

### Synthesis of rac-(1S*,2S*)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-28)

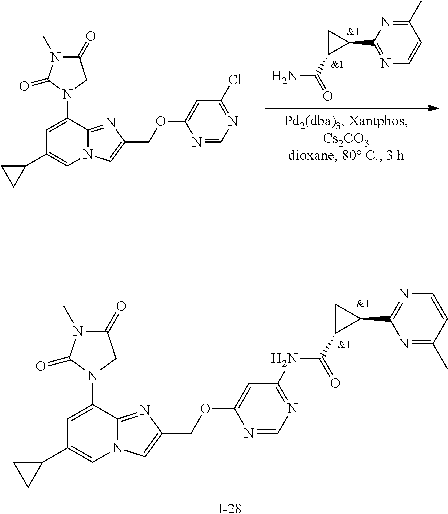

To a mixture of 1-(2-(((6-chloropyrimidin-4-yl)oxy)methyl)-6-cyclopropylimidazo[1,2-a]pyridin-8-yl)-3-methylimidazolidine-2,4-dione (50 mg, 0.12 mmol), rac-(1S*,2S*)-2-(4-methylpyrimidin-2-yl)cyclopropane-1-carboxamide (21 mg, 0.12 mmol), and Cs₂CO₃ (79 mg, 0.24 mmol) in dioxane (4 mL) was added Pd₂(dba)₃ (22 mg, 0.024 mmol) and XantPhos (28 mg, 0.048 mmol). The reaction mixture was stirred at 80° C. for 3 h under N₂. The reaction mixture was cooled to room temperature, filtered through Celite®, and the filter cake was washed with DCM/MeOH (10/1, 80 mL). The filtrate was concentrated to give the crude, which was purified by preparative TLC (eluent: DCM/MeOH=10/1) to give the title compound as a mixture of enantiomers (7 mg, 10.5%) as a white solid. ESI-MS [M+H]+: 554.2. ¹H NMR (400 MHz, DMSO) δ=11.21 (s, 1H), 8.55 (s, 1H), 8.53 (d, J=5.1, 1H), 8.28 (s, 1H), 7.95 (s, 1H), 7.49 (s, 1H), 7.38 (d, J=1.3, 1H), 7.21 (d, J=5.1, 1H), 5.48 (s, 2H), 4.89 (s, 2H), 2.97 (s, 3H), 2.68-2.65 (m, 1H), 2.58-2.54 (m, 1H), 2.41 (s, 3H), 1.99-1.93 (m, 1H), 1.61-1.52 (m, 2H), 0.99-0.91 (m, 2H), 0.70-0.62 (m, 2H).

The mixture was separated using SFC (Daicel CHIRALPAK OD-H 250 mm×20 mm I.D. CO2/MeOH (0.2% NH4·OH)=60/40, 50 g/min, 35° C.) to give two enantiomers: (1S,2S)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide and (1R,2R)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide.

First eluting isomer, (1R,2R)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-29): ESI-MS (M+H)+: 554.2, ¹H NMR (400 MHz, DMSO) δ=11.20 (s, 1H), 8.60-8.46 (m, 2H), 8.28 (s, 1H), 7.95 (s, 1H), 7.49 (s, 1H), 7.38 (s, 1H), 7.22 (d, J=5.1, 1H), 5.49 (s, 2H), 4.89 (s, 2H), 2.97 (s, 3H), 2.69-2.65 (m, 1H), 2.58-2.55 (m, 1H), 2.42 (s, 3H), 1.96-1.92 (m, 1H), 1.60-1.52 (m, 2H), 1.00-0.92 (m, 2H), 0.71-0.62 (m, 2H). RT=1.76 min, 100.0% e.e.

Second eluting isomer, (1S,2S)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-30): ESI-MS (M+H)+: 554.2, ¹H NMR (400 MHz, DMSO) δ=11.20 (s, 1H), 8.58-8.49 (m, 2H), 8.28 (s, 1H), 7.95 (s, 1H), 7.49 (s, 1H), 7.38 (d, J=1.4, 1H), 7.22 (d, J=5.1, 1H), 5.48 (s, 2H), 4.89 (s, 2H), 2.97 (s, 3H), 2.68-2.65 (m, 1H), 2.59-2.56 (m, 1H), 2.42 (s, 3H), 1.96-1.93 (m, 1H), 1.60-1.53 (m, 2H), 0.98-0.93 (m, 2H), 0.69-0.63 (m, 2H). RT=2.33 min, 100.0% e.e.

### Example 31

### Synthesis of rac-(1S*,2S*)—N-(6-((8-(3-(2-(benzyloxy)ethyl)-2,4-dioxoimidazolidin-1-yl)-6-cyclopropylimidazo[1,2-a]pyridin-2-yl)methoxy)pyrimidin-4-yl)-2-(4-methylpyrimidin-2-yl cyclopropane-1-carboxamide (I-31)

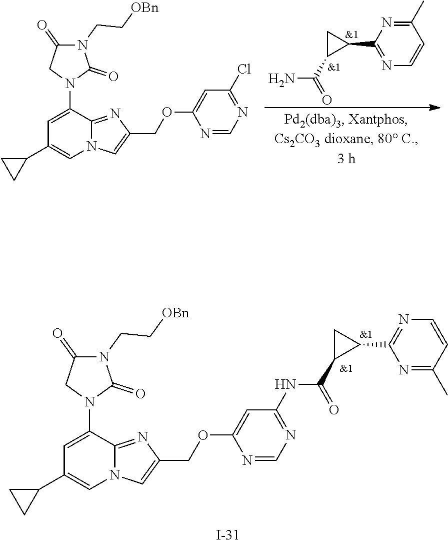

The title compound was prepared in a similar manner to rac-(1S*,2S*)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide from 3-(2-(benzyloxy)ethyl)-1-(2-(((6-chloropyrimidin-4-yl)oxy)methyl)-6-cyclopropylimidazo[1,2-a]pyridin-8-yl)imidazolidine-2,4-dione and rac-(1S*,2S*)-2-(4-methylpyrimidin-2-yl)cyclopropane-1-carboxamide. ESI-MS (M+H)+: 674.3, ¹H NMR (400 MHz, DMSO) δ 11.22 (s, 1H), 8.57 (d, J=0.8 Hz, 1H), 8.53 (d, J=5.1 Hz, 1H), 8.40 (s, 1H), 8.06 (s, 1H), 7.50 (d, J=0.8 Hz, 1H), 7.45 (s, 1H), 7.38-7.29 (m, 4H), 7.29-7.24 (m, 1H), 7.22 (d, J=5.1 Hz, 1H), 5.53 (s, 2H), 4.86 (s, 2H), 4.51 (s, 2H), 3.74-3.71 (m, 2H), 3.66-3.63 (2H), 2.69-2.65 (m, 1H), 2.61-2.53 (m, 1H), 2.42 (s, 3H), 2.01-1.98 (m, 1H), 1.60-1.52 (m, 2H), 1.09-0.85 (m, 2H), 0.71-0.69 (m, 2H).

### Example 32

### Synthesis of (1S,2S)—N-(6-((6-cyclopropyl-8-(2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-32)

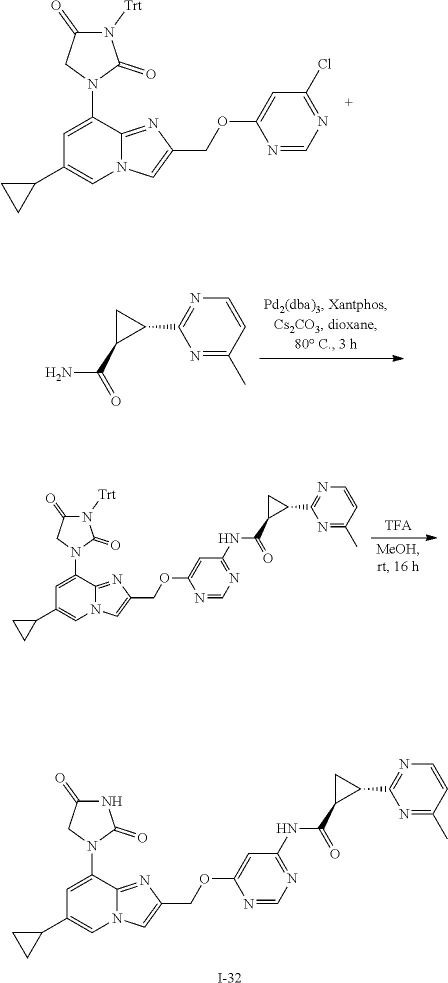

Synthesis of (1S,2S)—N-(6-((6-cyclopropyl-8-(2,4-dioxo-3-tritylimidazolidin-1-yl)imidazo[1,2-a]pyridin-2-yl)methoxy)pyrimidin-4-yl)-2-(4-methylpyrimidin-2-yl)cyclopropane-1-carboxamide. The title compound was prepared in a similar manner to rac-(1S*,2S*)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide from 1-(2-(((6-chloropyrimidin-4-yl)oxy)methyl)-6-cyclopropylimidazo[1,2-a]pyridin-8-yl)-3-tritylimidazolidine-2,4-dione dione and (1S,2S)-2-(4-methylpyrimidin-2-yl)cyclopropane-1-carboxamide. ESI-MS (M+H)+: 782.2

Synthesis of (1S,2S)—N-(6-((6-cyclopropyl-8-(2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-32). To a solution of (1S,2S)—N-(6-((6-cyclopropyl-8-(2,4-dioxo-3-tritylimidazolidin-1-yl)imidazo[1,2-a]pyridin-2-yl)methoxy)pyrimidin-4-yl)-2-(4-methylpyrimidin-2-yl)cyclopropane-1-carboxamide (30 mg, 0.038 mmol) in MeOH (3 mL) was added TFA (3 mL). The resulting mixture was stirred at room temperature for 1 h. The reaction mixture was concentrated in vacuo and the residue was neutralized with NH₃ in MeOH (7M, 1 mL). The resulting solution was concentrated in vacuo and purified by preparative TLC (eluent: MeOH/DCM=1/10) to afford (1S,2S)—N-(6-((6-cyclopropyl-8-(2,4-dioxoimid azolidin-1-yl)imidazo [1,2-a]pyridin-2-yl)methoxy)pyrimidin-4-yl)-2-(4-methylpyrimidin-2-yl)cyclopropane-1-carboxamide (10 mg, 48%) as a pale solid. ESI-MS (M+H)+: 540.2, ¹H NMR (400 MHz, DMSO) δ 11.35 (s, 1H), 11.20 (s, 1H), 8.56-8.52 (m, 2H), 8.27 (s, 1H), 7.95 (s, 1H), 7.49 (d, J=0.9 Hz, 1H), 7.39 (s, 1H), 7.22-7.21 (m, 1H), 5.49 (s, 2H), 4.85 (s, 2H), 2.67-2.65 (m, 1H), 2.58-2.55 (m, 1H), 2.42 (s, 3H), 1.98-1.94 (m, 1H), 1.59-1.52 (m, 2H), 0.98-0.93 (m, 2H), 0.69-0.65 (m, 2H).

### Example 33

### Synthesis of rac-(1S*,2S*)—N-(6-((6-cyclopropyl-8-(3-(2-hydroxyethyl)-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (I-33)

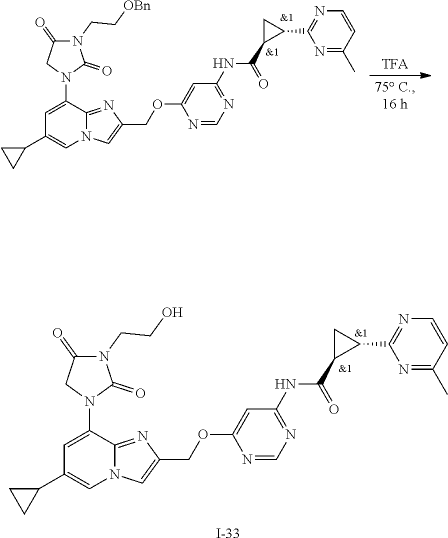

A solution of N-(6-((8-(3-(2-(benzyloxy)ethyl)-2,4-dioxoimidazolidin-1-yl)-6-cyclopropylimidazo[1,2-a]pyridin-2-yl)methoxy)pyrimidin-4-yl)-2-(4-methylpyrimidin-2-yl)cyclopropane-1-carboxamide (25 mg, 0.037 mmol) in TFA (3 mL) was stirred at 70° C. for 16 h. The solution was cooled to room temperature and concentrated in vacuo, and the residue was neutralized with NH₃ in MeOH (7M, 1 mL). The resulting mixture was concentrated in vacuo and purified by preparative HPLC to give N-(6-((6-cyclopropyl-8-(3-(2-hydroxyethyl)-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (10 mg, 46%) as a white solid. ESI-MS (M+H)+: 584.2, ¹H NMR (400 MHz, DMSO) δ 11.20 (s, 1H), 8.56 (d, J=0.9 Hz, 1H), 8.53 (d, J=5.1 Hz, 1H), 8.27 (d, J=0.9 Hz, 1H), 7.95 (s, 1H), 7.49 (d, J=0.9 Hz, 1H), 7.38 (d, J=1.4 Hz, 1H), 7.22 (d, J=5.0 Hz, 1H), 5.49 (s, 2H), 4.90 (d, J=10.3 Hz, 3H), 3.56 (d, J=3.7 Hz, 4H), 2.67-2.64 (m, 1H), 2.59-2.54 (m, 1H), 2.42 (s, 3H), 2.00-1.94 (m, 1H), 1.61-1.50 (m, 2H), 1.06-0.87 (m, 2H), 0.76-0.60 (m, 2H).

### Synthesis of 8-bromo-2-(((2-chloro-6-methylpyridin-4-yl)oxy)methyl)-6-cyclopropylimidazo[1,2-a]pyridine

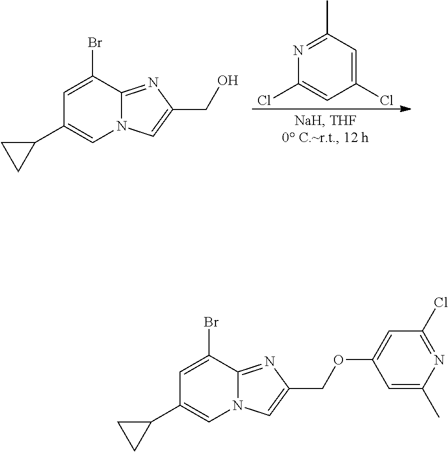

To mixture of (8-bromo-6-cyclopropylimidazo[1,2-a]pyridin-2-yl)methanol (532 mg, 2.0 mmol) in dry THF (10.0 mL) was added NaH (120 mg, 3.0 mmol) at 0° C. After stirring at 0° C. for 0.5 h, a solution of 2,4-dichloro-6-methylpyridine (483 mg, 3.0 mmol) was added, and the reaction mixture was stirred at room temperature for another 12 h. The reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by column chromatography (eluent: DCM/MeOH=40/1) to give 8-bromo-2-(((2-chloro-6-methylpyridin-4-yl)oxy)methyl)-6-cyclopropylimidazo[1,2-a]pyridine (160 mg, 20%) as a yellow solid. ESI-MS [M+H]⁺: 394.1.

### Synthesis of 1-(2-(((2-chloro-6-methylpyridin-4-yl)oxy)methyl)-6-cyclopropylimidazo[1,2-a]pyridin-8-yl)-3-methylimidazolidine-2,4-dione

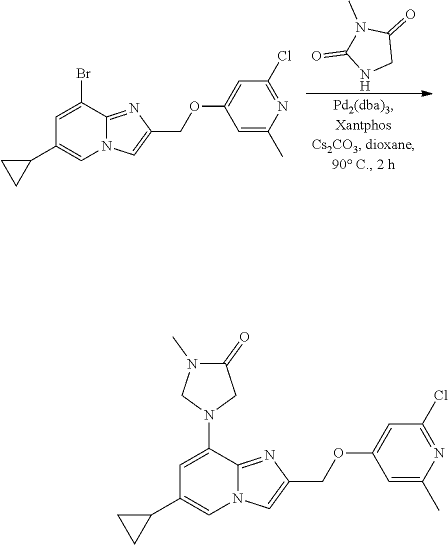

To a mixture of 8-bromo-2-(((2-chloro-6-methylpyridin-4-yl)oxy)methyl)-6-cyclopropylimidazo[,2-a]pyridine (50 mg, 0.13 mmol), 3-methylimidazolidine-2,4-dione (17.4 mg, 0.15 mmol), and Cs₂CO₃ (124 mg, 0.38 mmol) in dioxane (5 mL) were added Pd₂(dba)₃ (12 mg, 0.013 mmol) and Xantphos (15 mg, 0.026 mmol). After stirring at 90° C. for 2 h under N₂, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=30/1) to give 1-(2-(((2-chloro-6-methylpyridin-4-yl)oxy)methyl)-6-cyclopropylimidazo[1,2-a]pyridin-8-yl)-3-methylimidazolidine-2,4-dione (30 mg, 56%) as a white solid. ESI-MS [M+H]⁺: 426.2.

The compounds in Table 5 were made in a similar manner to 1-(2-(((2-chloro-6-methylpyridin-4-yl)oxy)methyl)-6-cyclopropylimidazo[1,2-a]pyridin-8-yl)-3-methylimidazolidine-2,4-dione from appropriate starting materials.

TABLE 5

| | Analytical |
|---|---|
| Intermediate | Data |
| 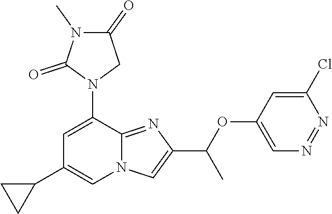 [SMILES: CC(Oc1cnnc(Cl)c1)c1cn2cc(C3CC3)cc(N3CC(=O)N(C)C3=O)c2n1] | ESI-MS [M + H]⁺: 427.1. |
| 1-(2-(1-((6-chloropyridazin-4-yl)oxy)ethyl)-6- | |
| cyclopropylimidazo[1,2- | |
| a]pyridin-8-yl)-3-methylimidazolidine-2,4-dione | |
| 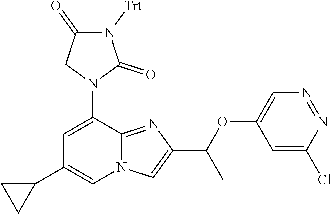 | ESI-MS [M + H]⁺: 655.2. |
| 1-(2-(1-((6-chloropyridazin-4-yl)oxy)ethyl)-6- | |
| cyclopropylimidazo[1,2-a]pyridin-8-yl)-3- | |
| tritylimidazolidine-2,4-dione | |
| 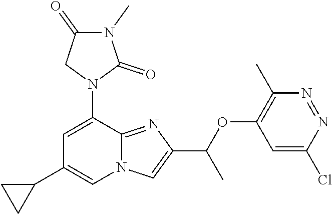 [SMILES: Cc1nnc(Cl)cc1OC(C)c1cn2cc(C3CC3)cc(N3CC(=O)N(C)C3=O)c2n1] | ESI-MS [M + H]⁺: 441.1. |
| 1-(2-(1-((6-chloro-3-methylpyridazin-4-yl)oxy)ethyl)-6- | |
| cyclopropylimidazo[1,2-a]pyridin-8-yl)-3- | |
| methylimidazolidine-2,4-dione | |
| 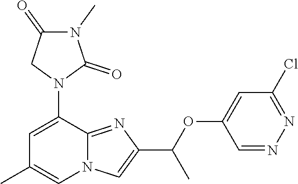 [SMILES: Cc1cc(N2CC(=O)N(C)C2=O)c2nc(C(C)Oc3cnnc(Cl)c3)cn2c1] | ESI-MS [M + H]⁺: 401.2. |
| 1-(2-(1-((6-chloropyridazin-4-yl)oxy)ethyl)- | |
| 6-methylimidazo[1,2-a]pyridin-8-yl)-3- | |
| methylimidazolidine-2,4-dione | |
| 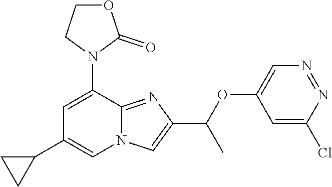 [SMILES: CC(Oc1cnnc(Cl)c1)c1cn2cc(C3CC3)cc(N3CCOC3=O)c2n1] | ESI-MS [M + H]⁺: 400.2. |
| 3-(2-(1-((6-chloropyridazin-4-yl)oxy)ethyl)-6- | |
| cyclopropylimidazo[1,2-a]pyridin-8-yl)oxazolidin-2-one | |
| 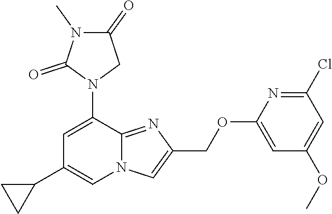 [SMILES: COc1cc(Cl)nc(OCc2cn3cc(C4CC4)cc(N4CC(=O)N(C)C4=O)c3n2)c1] | ESI-MS [M + H]⁺: 442.1. |
| 1-(2-(((6-chloro-4-methoxypyridin-2-yl)oxy)methyl)-6- | |
| cyclopropylimidazo[1,2-a]pyridin-8-yl)-3- | |
| methylimidazolidine-2,4-dione | |

### Example 34

### Synthesis of (1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide (I-34)

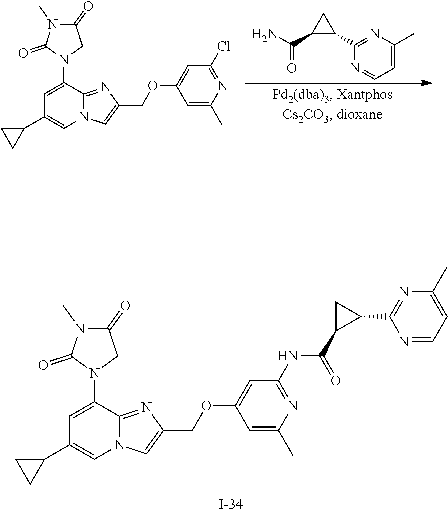

To a mixture of 1-(2-(((2-chloro-6-methylpyridin-4-yl)oxy)methyl)-6-cyclopropylimidazo[1,2-a]pyridin-8-yl)-3-methylimidazolidine-2,4-dione (40 mg, 0.094 mmol), (1S,2S)-2-(4-methylpyrimidin-2-yl)cyclopropane-1-carboxamide (20 mg, 0.11 mmol), and Cs₂CO₃ (92 mg, 0.28 mmol) in dioxane (3 mL) were added Pd₂(dba)₃ (8.6 mg, 0.0094 mmol) and Xantphos (11 mg, 0.028 mmol). After stirring at 80° C. for 2 h under N₂, the reaction mixture was cooled to room temperature and filtered through Celite®. The filter cake was washed with DCM/MeOH (10/1, 20 mL) and the filtrate was concentrated to give the crude, which was purified by preparative HPLC to give (1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide (13 mg, 25%) as a white solid. ESI-MS [M+H]+: 567.2. ¹H NMR (400 MHz, DMSO) δ 10.76 (s, 1H), 8.50 (dd, J=5.1, 2.5 Hz, 1H), 8.27 (s, 1H), 7.94 (d, J=2.3 Hz, 1H), 7.65 (s, 1H), 7.37 (s, 1H), 7.26-7.09 (m, 1H), 6.71 (s, 1H), 5.21 (s, 2H), 4.89 (d, J=1.4 Hz, 2H), 2.95 (s, 3H), 2.60-2.58 (m, 2H), 2.39 (s, 3H), 2.31 (s, 3H), 1.99-1.89 (m, 1H), 1.49-1.47 (m, 2H), 0.98-0.88 (m, 2H), 0.69-0.60 (m, 2H).

The compounds in Table 6 were prepared in a similar manner to (1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide from (1S,2S)-2-(4-methylpyrimidin-2-yl)cyclopropane-1-carboxamide and an appropriate coupling partner.

TABLE 6

| | | Coupling Partner / |
|---|---|---|
| Example | Compound | Analytical Data |
| Example 35 | 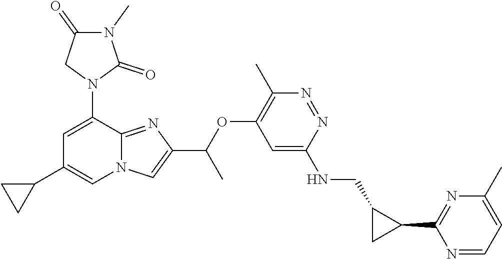 | 1-(2-(1-((6-chloro-3-methylpyridazin-4- yl)oxy)ethyl)-6-cyclopropylimidazo[1,2- a]pyridin-8-yl)-3-methylimidazolidine-2,4- dione ESI-MS [M + H]+: 582.2, ¹H NMR (400 MHz, DMSO) δ 11.21 (s, 1H), 8.52 (t, J = 5.2 Hz, 1H), 8.22 (s, 1H), 8.16 (s, 1H), 7.92 (d, J = 3.6 Hz, 1H), 7.41 (d, J = 10.2 Hz, 1H), 7.20 (t, J = 4.8 Hz, 1H), 5.78 − 5.59 (m, 1H), 5.10 − 4.92 (m, 2H), 2.99 (d, J = 8.2 Hz, 3H), 2.62 − 2.60 (m, 2H), 2.47 − 2.36 (m, 6H), 1.99 − 1.91 (m, 1H), 1.75 (d, J = 6.4 Hz, 3H), 1.58 − 1.45 (m, 2H), 1.01 − 0.89 (m, 2H), 0.73 − 0.58 (m, 2H). |
| | (1S,2S)-N-(5-(1-(6- | |
| | cyclopropyl-8-(3-methyl- | |
| | 2,4-dioxoimidazolidin-1- | |
| | yl)imidazo[1,2-α]pyridin-2- | |
| | yl)ethoxy)-6- | |
| | methylpyridazin-3-yl)-2-(4- | |
| | methylpyrimidin-2- | |
| | yl)cyclopropane-1- | |
| | carboxamide (I-35) | |
| Example 36 | 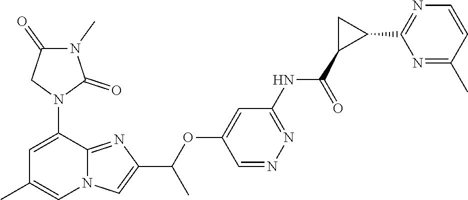 (1S,2S)-N-(5-(1-(6-methyl- | 1-(2-(1-((6-chloropyridazin-4- yl)oxy)ethyl)-6-methylimidazo[1,2- a]pyridin-8-yl)-3-methylimidazolidine- 2,4-dione ESI-MS [M + H]+: 542.2, ¹H NMR (400 MHz, DMSO) 8 11.32 (s, 1H), 8.73 − 8.72 (m, 1H), 8.54 − 8.52 (m, 1H), 8.21 − 8.16 (m, 2H), 7.99 (d, J = 2.1 Hz, 1H), 7.50 − 7.48 (m, 1H), 7.22 − 7.20 (m, 1H), 5.82 − 5.77 (m, 1H), 5.02 − 4.96 (m, 2H), 2.99 (d, J = 7.1 Hz, 3H), 2.56 − 2.55 (m, 1H), 2.52 − 2.52 (m, 1H), 2.42 (d, J = 3.8 Hz, 3H), 2.27 (s, 3H), 1.74 (d, J = 6.4 Hz, 3H), 1.57 − 1.51 (m, 2H). |
| | 8-(3-methyl-2,4- | |
| | dioxoimidazolidin-1- | |
| | yl)imidazo[1,2-α]pyridin-2- | |
| | yl)ethoxy)pyridazin-3-yl)-2- | |
| | (4-methylpyrimidin-2- | |
| | yl)cyclopropane-1- | |
| | carboxamide (I-36) | |
| Example 37 | 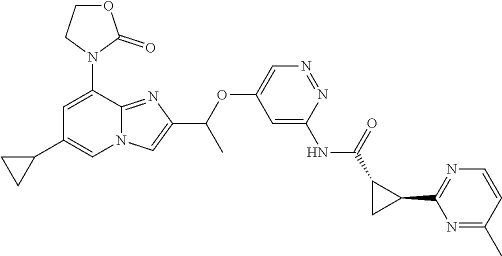 [SMILES: Cc1ccnc([C@H]2C[C@@H]2C(=O)Nc2cc(OC(C)c3cn4cc(C5CC5)cc(N5CCOC5=O)c4n3)cnn2)n1] | 3-(2-(1-((6-chloropyridazin-4- yl)oxy)ethyl)-6-cyclopropylimidazo[1,2- a]pyridin-8-yl)oxazolidin-2-one ESI-MS [M + H]+: 541.2, ¹H NMR (400 MHz, DMSO) δ 11.34 (s, 1H), 8.75 − 8.74(m, 1H), 8.53 (d, J = 5.1 Hz, 1H), 8.24 − 8.23 (m, 1H), 8.10 − 8.08 (m, 1H), 7.92 (d, J = 2.9 Hz, 1H), 7.29 − 7.28 (m, 1H), 7.22 (d, J = 5.1 Hz, 1H), 5.84 − 5.79 (m, 1H), 4.53 − 4.43 (m, 4H), 2.56 − 2.55 (m, 1H), 2.52 − 2.52 (m, 1H), 2.42 (s, 3H), 1.97 − 1.93 (m, 1H), 1.73 (d, J = 6.4 Hz, 3H), 1.59 − 1.51 (m, 2H), 0.96 − 0.91 (m, 2H), 0.67 − 0.63 (m, 2H). |
| | (1S,2S)-N-(5-(1-(6- | |
| | cyclopropyl-8-(2- | |
| | oxooxazolidin-3- | |
| | yl)imidazo[1,2-α]pyridin-2- | |
| | yl)ethoxy)pyridazin-3-yl)-2- | |
| | (4-methylpyrimidin-2- | |
| | yl)cyclopropane-1- | |
| | carboxamide (I-37) | |
| Example 38 | 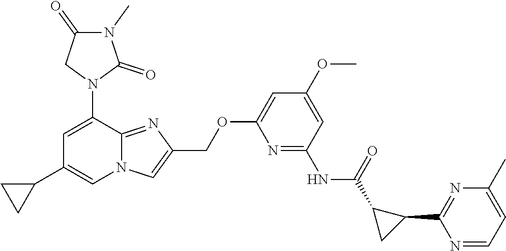 [SMILES: COc1cc(NC(=O)[C@H]2C[C@@H]2c2nccc(C)n2)nc(OCc2cn3cc(C4CC4)cc(N4CC(=O)N(C)C4=O)c3n2)c1] | 1-(2-(((6-chloro-4-methoxypyridin-2- yl)oxy)methyl)-6-cyclopropylimidazo[1,2- a]pyridin-8-yl)-3-methylimidazolidine-2,4- dione ESI-MS [M + H]+: 583.2. ¹H NMR (400 MHz, DMSO) δ 10.65 (s, 1H), 8.53 (d, J = 5.1 Hz, 1H), 8.27 (s, 1H), 7.93 (s, 1H), 7.36 (d, J = 1.5 Hz, 2H), 7.21 (d, J = 5.2 Hz, 1H), 6.12 (d, J = 1.9 Hz, 1H), 5.39 (s, 2H), 4.88 (s, 2H), 3.78 (s, 3H), 2.97 (s, 3H), 2.52-2.51 (m, 2H), 2.42 (s, 3H), 1.98- 1.92 (m, 1H), 1.67 − 1.50 (m, 2H), 0.97- 0.92 (m, 2H), 0.73 − 0.60 (m, 2H). |
| | (1S,2S)-N-(6-((6- | |
| | cyclopropyl-8-(3-methyl- | |
| | 2,4-dioxoimidazolidin-1- | |
| | yl)imidazo[1,2-α]pyridin-2- | |
| | yl)methoxy)-4- | |
| | methoxypyridin-2-yl)-2-(4- | |
| | methylpyrimidin-2- | |
| | yl)cyclopropane-1- | |
| | carboxamide (I-38) | |

### Example 39

### Synthesis of (1S,2S)—N-(5-(1-(6-cyclopropyl-8-(2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-39)

### Synthesis of (1S,2S)—N-(5-(1-(6-cyclopropyl-8-(2,4-dioxo-3-tritylimidazolidin-1-yl)imidazo[1,2-a]pyridin-2-yl)ethoxy)pyridazin-3-yl)-2-(4-methylpyrimidin-2-yl)cyclopropane-1-carboxamide

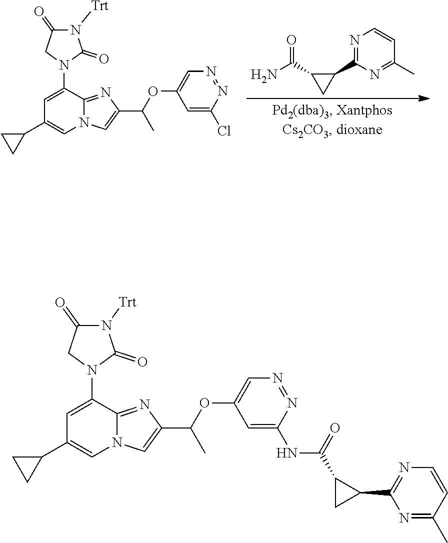

The title compound was prepared in a similar manner to (1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide from (1S,2S)-2-(4-methylpyrimidin-2-yl)cyclopropane-1-carboxamide and 1-(2-(1-((6-chloropyridazin-4-yl)oxy)ethyl)-6-cyclopropylimidazo[1,2-a]pyridin-8-yl)-3-tritylimidazolidine-2,4-dione.

### Synthesis of (1S,2S)—N-(5-(1-(6-cyclopropyl-8-(2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-39)

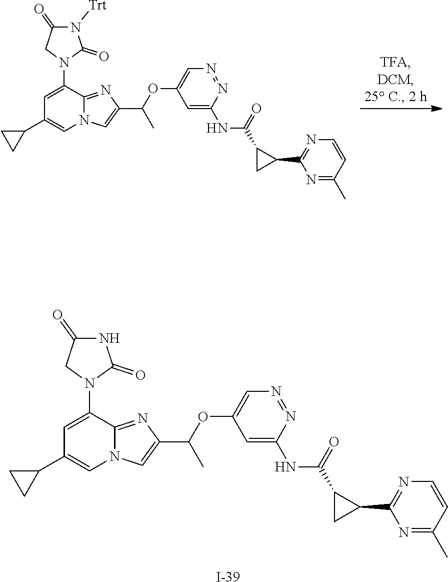

To a mixture of (1S,2S)—N-(5-(1-(6-cyclopropyl-8-(2,4-dioxo-3-tritylimidazolidin-1-yl)imidazo[1,2-a]pyridin-2-yl)ethoxy)pyridazin-3-yl)-2-(4-methylpyrimidin-2-yl)cyclopropane-1-carboxamide (100 mg, 0.12 mmol) in DCM (2 mL) was added TFA (1 mL). After stirring the reaction mixture at 25° C. for 2 h, the reaction mixture was diluted with DCM (30 mL) and washed with saturated aq·NaHCO₃ (10 mL), H₂O (10 mL), and brine (10 mL). The organic extract was dried over anhydrous Na₂SO₄ and concentrated to give the crude product, which was purified by preparative TLC (eluent: DCM/MeOH=10/1) to give (1S,2S)—N-(5-(1-(6-cyclopropyl-8-(2,4-dioxo-3-tritylimidazolidin-1-yl)imidazo[1,2-a]pyridin-2-yl)ethoxy)pyridazin-3-yl)-2-(4-methylpyrimidin-2-yl)cyclopropane-1-carboxamide (30 mg, 43%) as a yellow solid. ESI-MS [M+H]+: 554.2, ¹H NMR (400 MHz, DMSO) δ 11.40-11.27 (m, 2H), 8.75-8.54 (m, 1H), 8.54-8.52 (m, 1H), 8.22-8.21 (m, 1H), 8.16-8.07 (m, 1H), 7.92 (d, J=2.4 Hz, 1H), 7.41-7.32 (m, 1H), 7.22-7.20 (m, 1H), 5.81-5.80 (m, 1H), 4.91 (d, J=7.0 Hz, 2H), 2.65-2.62 (m, 1H), 2.58-2.56 (m, 1H), 2.42 (d, J=3.2 Hz, 3H), 1.99-1.91 (m, 1H), 1.74-1.72 (m, 3H), 1.60-1.51 (m, 2H), 0.94-0.93 (m, 2H), 0.66-0.65 (m, 2H).

### Examples 40-42

### Synthesis of (1S,2S)—N-(5-(1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-40)

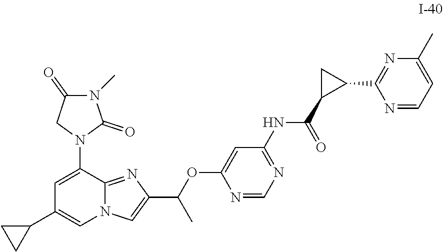 [SMILES: Cc1ccnc([C@H]2C[C@@H]2C(=O)Nc2cc(OC(C)c3cn4cc(C5CC5)cc(N5CC(=O)N(C)C5=O)c4n3)ncn2)n1]

The title compound was prepared in a similar manner to (1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide as a mixture of diastereomers. ESI-MS [M+H]+: 568.3. ¹H NMR (400 MHz, DMSO) δ 11.32 (s, 1H), 8.73 (d, J=2.6 Hz, 1H), 8.56-8.50 (m, 1H), 8.26-8.21 (m, 1H), 8.18-8.14 (m, 1H), 7.94 (d, J=3.1 Hz, 1H), 7.43-7.37 (m, 1H), 7.24-7.19 (m, 1H), 5.85-5.73 (m, 1H), 5.06-4.91 (m, 2H), 2.99 (d, J=7.2 Hz, 3H), 2.58-2.56 (m, 2H), 2.42 (d, J=3.7 Hz, 3H), 2.00-1.91 (m, 1H), 1.73 (d, J=6.4 Hz, 3H), 1.58-1.50 (m, 2H), 0.98-0.92 (m, 2H), 0.68-0.61 (m, 2H).

The mixture was separated using SFC 80 (Daicel CHIRALPAK AS-H 20×250 mm, 5.0 μm, CO2/MeOH (0.2% NH3 (7M in MeOH))=65/35, 50 g/min, 35° C.) to give two diastereomers: (1S,2S)—N-(5-((R)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide and (1S,2S)—N-(5-((S)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide

First eluting isomer, (1S,2S)—N-(5-((S)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-41): ESI-MS [M+H]+: 568.2, ¹H NMR (400 MHz, DMSO) δ 11.32 (s, 1H), 8.73 (d, J=2.7 Hz, 1H), 8.52 (d, J=5.1 Hz, 1H), 8.25-8.22 (m, 1H), 8.16 (d, J=2.7 Hz, 1H), 7.94 (s, 1H), 7.41 (d, J=1.4 Hz, 1H), 7.21 (d, J=5.1 Hz, 1H), 5.83-5.75 (m, 1H), 5.06-4.90 (m, 2H), 3.00 (s, 3H), 2.53-2.51 (m, 2H), 2.41 (s, 3H), 1.99-1.92 (m, 1H), 1.73 (d, J=6.4 Hz, 3H), 1.57-1.51 (m, 2H), 0.97-0.92 (m, 2H), 0.67-0.63 (m, 2H). RT=2.21 min, 100.0% e.e.

Second eluting isomer, (1S,2S)—N-(5-((R)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide (I-42): ESI-MS [M+H]+: 568.3, ¹H NMR (400 MHz, DMSO) δ 11.32 (s, 1H), 8.73 (d, J=2.7 Hz, 1H), 8.53 (d, J=5.1 Hz, 1H), 8.25-8.21 (m, 1H), 8.16 (d, J=2.6 Hz, 1H), 7.94 (s, 1H), 7.39 (d, J=1.4 Hz, 1H), 7.22 (d, J=5.1 Hz, 1H), 5.82-5.77 (m, 1H), 4.95 (s, 2H), 2.98 (s, 3H), 2.55-2.53 (m, 2H), 2.42 (s, 3H), 1.98-1.92 (m, 1H), 1.73 (d, J=6.4 Hz, 3H), 1.56-1.50 (m, 2H), 0.98-0.91 (m, 2H), 0.67-0.60 (m, 2H). RT=3.33 min, 100.0% e.e.

### Synthesis of rac-(1S*,2S*)-2-(3-chloro-2-fluorophenyl)-N-(6-chloropyrimidin-4-yl)cyclopropane-1-carboxamide

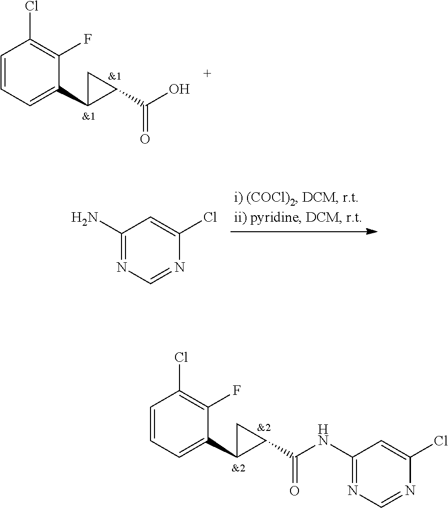 [SMILES: O=C(O)[C@H]1C[C@@H]1c1cccc(Cl)c1F ; Nc1cc(Cl)ncn1 ; O=C(Nc1cc(Cl)ncn1)[C@H]1C[C@@H]1c1cccc(Cl)c1F]

Oxalyl chloride (0.31 mL, 3.6 mmol) and DMF (2 drops) were added to a solution of rac-(1S*,2S*)-2-(3-chloro-2-fluorophenyl)cyclopropane-1-carboxylic acid (0.26 g, 1.2 mmol) in DCM (5 mL). The mixture was stirred at room temperature for 2 h then concentrated in vacuo. Residual oxalyl chloride was removed by azeotroping with toluene. The residue was redissolved in DCM (10 mL) and cooled to 0° C. A suspension of 6-chloropyrimidin-4-amine (0.16 g, 1.2 mmol) and DIPEA (0.63 mL, 3.6 mmol) in DCM (15 mL) was added portionwise to the mixture. The mixture was allowed to warm to room temperature and stirred for 18 h. The mixture was washed with a solution of K₂CO₃ (sat·aq·, 15 mL), dried (MgSO₄), filtered, and concentrated in vacuo. The residue was purified by column chromatography on silica gel, eluting with 5% EtOAc in DCM to give the title compound (0.15 g, 40%) as an orange-brown sticky residue. ESI-MS (M+H)+: 326, ¹H NMR (400 MHz, CDCl₃) δ 8.64 (s, 1H), 8.37 (s, 1H), 8.25 (s, 1H), 7.30-7.25 (m, 1H), 7.03 (m, 1H), 6.97-6.90 (m, 1H), 2.79-2.72 (m, 1H), 1.92-1.86 (m, 1H), 1.80-1.75 (m, 1H), 1.56-1.48 (m, 1H).

Using a similar procedure to that used for the synthesis of rac-(1S*,2S*)-2-(3-chloro-2-fluorophenyl)-N-(6-chloropyrimidin-4-yl)cyclopropane-1-carboxamide, the compounds in Table 7 were prepared from 6-chloropyrimidin-4-amine and an appropriate acid coupling partner.

TABLE 7

| | Coupling Partner/ |
|---|---|
| Structure | Analytical Data |
| 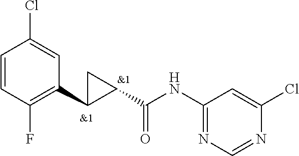 [SMILES: O=C(Nc1cc(Cl)ncn1)[C@H]1C[C@@H]1c1cc(Cl)ccc1F] | rac-(1S*,2S*)-2-(5-chloro-2- fluorophenyl)cyclopropane-1-carboxylic acid ESI-MS (M + H)+: 326 ¹H NMR (400 MHz, CDCl₃) δ 8.64 (s, 1H), 8.25 (s, 2H), 7.21 − 7.15 (m, 1H), 7.02 − 6.95 (m, 2H), 2.76 − 2.69 (m, 1H), 1.90 − 1.74 (m, 2H), 1.54 − 1.48 (m, 1H). |
| rac-(1S*,2S*)-2-(5-chloro-2- | |
| fluorophenyl)-N-(6-chloropyrimidin-4- | |
| yl)cyclopropane-1-carboxamide | |
| 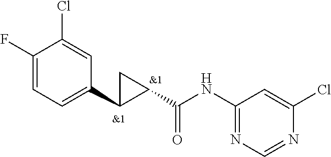 [SMILES: O=C(Nc1cc(Cl)ncn1)[C@H]1C[C@@H]1c1ccc(F)c(Cl)c1] | rac-(1S*,2S*)-2-(3-chloro-4- fluorophenyl)cyclopropane-1-carboxylic acid ESI-MS (M + H)+: 326 |
| rac-(1S*,2S*)-2-(3-chloro-4- | |
| fluorophenyl)-N-(6-chloropyrimidin-4- | |
| yl)cyclopropane-1-carboxamide | |
| 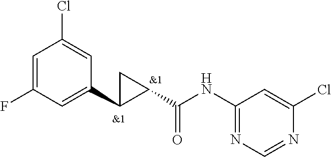 [SMILES: O=C(Nc1cc(Cl)ncn1)[C@H]1C[C@@H]1c1cc(F)cc(Cl)c1] | rac-(1S*,2S*)-2-(3-chloro-5- fluorophenyl)cyclopropane-1-carboxylic acid ESI-MS (M + H)+: 326 |
| rac-(1S*,2S*)-2-(3-chloro-5- | |
| fluorophenyl)-N-(6-chloropyrimidin-4- | |
| yl)cyclopropane-1-carboxamide | |
| 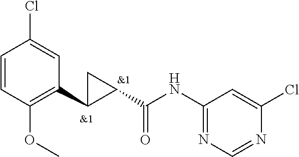 [SMILES: COc1ccc(Cl)cc1[C@H]1C[C@@H]1C(=O)Nc1cc(Cl)ncn1] | rac-(1S*,2S*)-2-(5-chloro-2- methoxyphenyl)cyclopropane-1-carboxylic acid ESI-MS (M + H)+: 337.9, ¹H NMR (400 MHz, CDCl₃) δ 8.63 (s, 1H), 8.33 (s, 1H), 8.26 (s, 1H), 7.21 − 7.15 (m, 1H), 6.94 − 6.91 (m, 1H), 6.79 (d, J = 8.6 Hz, 1H), 3.85 (s, 3H), 2.75 − 2.67 (m, 1H), 1.74 − 1.69 (m, 2H), 1.50 − 1.45 (m, 1H). |
| rac-(1S*,2S*)-2-(5-chloro-2- | |
| methoxyphenyl)-N-(6-chloropyrimidin- | |
| 4-yl)cyclopropane-1-carboxamide | |
| 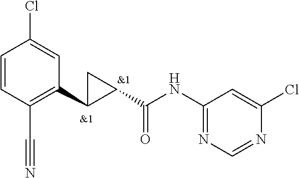 [SMILES: N#Cc1ccc(Cl)cc1[C@H]1C[C@@H]1C(=O)Nc1cc(Cl)ncn1] | rac-(1S*,2S*)-2-(5-chloro-2- cyanophenyl)cyclopropane-1-carboxylic acid ESI-MS (M + H)+: 331.0, ¹H NMR (400 MHz, DMSO) 8 11.61 (s, 1H), 8.79 (d, J = 1.0 Hz, 1H), 8.16 (d, J = 1.0 Hz, 1H), 7.89 (d, J = 8.4 Hz, 1H), 7.56 (dd, J = 2.1, 8.3 Hz, 1H), 7.46 (d, J = 2.1 Hz, 1H), 2.72 − 2.65 (m, 1H), 1.76 − 1.62 (m, 2H). 1H missing, presumed under DMSO peak. |
| rac-(1S*,2S*)-2-(5-chloro-2- | |
| cyanophenyl)-N-(6-chloropyrimidin-4- | |
| yl)cyclopropane-1-carboxamide | |
| 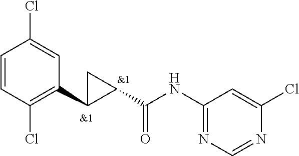 [SMILES: O=C(Nc1cc(Cl)ncn1)[C@H]1C[C@@H]1c1cc(Cl)ccc1Cl] | rac-(1S*,2S*)-2-(2,5-dichlorophenyl)cyclopropane-1- carboxylic acid ESI-MS (M + H)+: 342 |
| rac-(1S*,2S*)-N-(6-chloropyrimidin-4- | |
| yl)-2-(2,5-dichlorophenyl)cyclopropane- | |
| 1-carboxamide | |
| 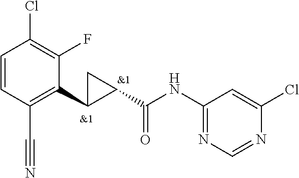 [SMILES: N#Cc1ccc(Cl)c(F)c1[C@H]1C[C@@H]1C(=O)Nc1cc(Cl)ncn1] | rac-(1S*,2S*)-2-(3-chloro-6-cyano-2- fluorophenyl)cyclopropane-1-carboxylic acid ESI-MS (M + H)+: 349.0 |
| rac-(1S*,2S*)-2-(3-chloro-6-cyano-2- | |
| fluorophenyl)-N-(6-chloropyrimidin-4- | |
| yl)cyclopropane-1-carboxamide | |
| 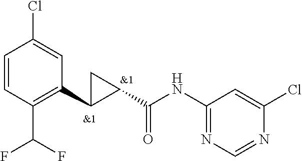 [SMILES: O=C(Nc1cc(Cl)ncn1)[C@H]1C[C@@H]1c1cc(Cl)ccc1C(F)F] | rac-(1S*,2S*)-2-(5-chloro-2- (difluoromethyl)phenyl)cyclopropane-1-carboxylic acid ESI-MS (M + H)+: 356.0, ¹H NMR (400 MHz, DMSO) δ 11.62 − 11.59 (m, 1H), 8.83 − 8.81 (m, 1H), 8.20 (s, 1H), 7.66 − 7.61 (m, 4H), 7.54 − 7.40 (m, 4H), 7.66 − 7.19 (m, 4H), 7.34 − 7.32 (m, 4H), 2.72 − 2.67 (m, 1H), 1.69 − 1.59 (m, 2H). 1H missing, presumed under DMSO peak. |
| rac-(1S*,2S*)-2-(5-chloro-2- | |
| (difluoromethyl)phenyl)-N-(6- | |
| chloropyrimidin-4-yl)cyclopropane-1- | |
| carboxamide | |
| 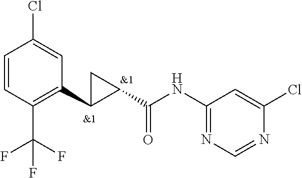 [SMILES: O=C(Nc1cc(Cl)ncn1)[C@H]1C[C@@H]1c1cc(Cl)ccc1C(F)(F)F] | rac-(1S*,2S*)-2-(5-chloro-2- (trifluoromethyl)phenyl)cyclopropane-1-carboxylic acid ESI-MS (M + H)+: 374 |
| rac-(1S*,2S*)-2-(5-chloro-2- | |
| (trifluoromethyl)phenyl)-N-(6- | |
| chloropyrimidin-4-yl)cyclopropane-1- | |
| carboxamide | |
| 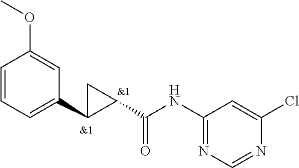 [SMILES: COc1cccc([C@H]2C[C@@H]2C(=O)Nc2cc(Cl)ncn2)c1] | rac-(1S*,2S*)-2-(3-methoxyphenyl)cyclopropane-1- carboxylic acid ESI-MS (M + H)+: 304.1 |
| rac-(1S*,2S*)-N-(6-chloropyrimidin-4- | |
| yl)-2-(3-methoxyphenyl)cyclopropane- | |
| 1-carboxamide | |
| 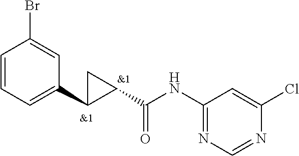 [SMILES: O=C(Nc1cc(Cl)ncn1)[C@H]1C[C@@H]1c1cccc(Br)c1] | rac-(1S*,2S*)-2-(3-bromophenyl)cyclopropane-1- carboxylic acid ESI-MS (M + H)+: 351.9, 353.9 |
| rac-(1S*,2S*)-2-(3-bromophenyl)-N-(6- | |
| chloropyrimidin-4-yl)cyclopropane-1- | |
| carboxamide | |

### Example 43

### Synthesis of rac-(1S*,2S*)-2-(3-chloro-2-fluorophenyl)-N-(6-(((6-cyclopropylimidazo[1,2-a]pyridin-2-yl)methyl)amino)pyrimidin-4-yl)cyclopropane-1-carboxamide (I-43)

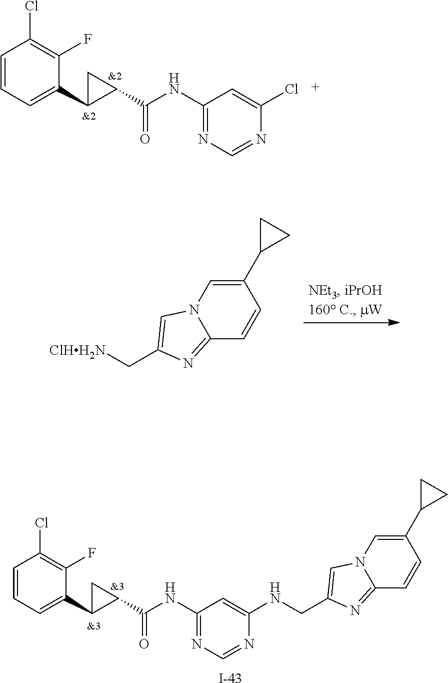

A mixture of rac-(1S*,2S*)-2-(3-chloro-2-fluorophenyl)-N-(6-chloropyrimidin-4-yl)cyclopropane-1-carboxamide (0.062 g, 0.19 mmol), (6-cyclopropylimidazo[1,2-a]pyridin-2-yl)methanamine (0.036 g, 0.19 mmol), and DIPEA (0.099 mL, 0.57 mmol) in iPrOH (2.0 mL) was stirred at 140° C. in a microwave for 1.5 h. The mixture was concentrated in vacuo. The residue was redissolved in DCM and washed with a solution of Na₂CO₃ (sat·aq·). The organic layer was concentrated in vacuo and the residue was purified by reverse phase preparative HPLC to give the title compound (0.035 g, 38%) as a beige solid. ESI-MS (M+H)+: 477.3, ¹H NMR (400 MHz, DMSO) δ 10.67 (s, 1H), 8.31 (s, 1H), 8.22 (s, 1H), 7.88 (br s, 1H), 7.62 (s, 1H), 7.49-7.44 (m, 1H), 7.38 (d, J=9.3 Hz, 1H), 7.29 (s, 1H), 7.22-7.12 (m, 2H), 6.97 (dd, J=1.8, 9.3 Hz, 1H), 4.58 (br s, 2H), 2.52-2.46 (m, 1H), 2.42-2.35 (m, 1H), 1.96-1.88 (m, 1H), 1.53-1.43 (m, 2H), 0.95-0.89 (m, 2H), 0.70-0.65 (m, 2H).

Using a similar procedure to that used for rac-(1S*,2S*)-2-(3-chloro-2-fluorophenyl)-N-(6-(((6-cyclopropylimidazo[1,2-a]pyridin-2-yl)methyl)amino)pyrimidin-4-yl)cyclopropane-1-carboxamide, the compounds in Table 8 were prepared from (6-cyclopropylimidazo[1,2-a]pyridin-2-yl)methanamine and appropriate coupling partner.

TABLE 8

| | | Coupling Partner/ | |
|---|---|---|---|
| Example | Compound | Analytical Data | |
| Example 44 | 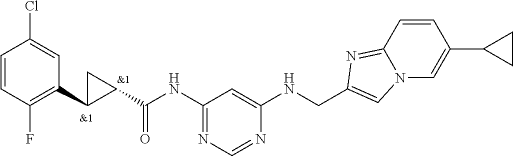 [SMILES: O=C(Nc1cc(NCc2cn3cc(C4CC4)ccc3n2)ncn1)[C@H]1C[C@@H]1c1cc(Cl)ccc1F] rac-(1S*,2S*)-2-(5-chloro-2- fluorophenyl)-N-(6-(((6- | rac-(1S*,2S*)-2-(5-chloro-2- fluorophenyl)-N-(6-chloropyrimidin- 4-yl)cyclopropane-1-carboxamide ESI-MS (M + H)+: 477.3, ¹H NMR (400 MHz, DMSO) δ 10.64 (s, 1H), 8.31 (s, 1H), 8.22 (s, 1H), 7.88 (br s, 1H), 7.62 (s, 1H), 7.40 − 7.31 (m, 2H), 7.31 − 7.22 (m, 3H), 6.97 (dd, J = 1.8, 9.3 Hz, 1H), 4.58 − 4.58 (br s, 2H), 2.47 − 2.38 (m, 2H), 1.96 − 1.88 (m, 1H), 1.53 − 1.44 (m, 2H), 0.95 − 0.89 (m, 2H), 0.70 − 0.65 (m, 2H). | |
| | cyclopropylimidazo[1,2-a]pyridin-2- | | |
| | yl)methyl)amino)pyrimidin-4- | | |
| | yl)cyclopropane-1-carboxamide (I-44) | | |
| Example 45 | 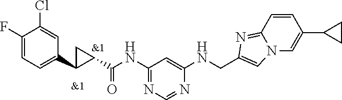 [SMILES: O=C(Nc1cc(NCc2cn3cc(C4CC4)ccc3n2)ncn1)[C@H]1C[C@@H]1c1ccc(F)c(Cl)c1] rac-(1S*,2S*)-2-(3-chloro-4- fluorophenyl)-N-(6-(((6- cyclopropylimidazo[1,2-a]pyridin-2- yl)methyl)amino)pyrimidin-4- yl)cyclopropane-1-carboxamide (I-45) | rac-(1S*,2S*)-2-(3-chloro-4- fluorophenyl)-N-(6-chloropyrimidin- 4-yl)cyclopropane-1-carboxamide ESI-MS (M + H)+: 477.3, ¹H NMR (400 MHz, DMSO) δ 10.61 (s, 1H), 8.34 (s, 1H), 8.21 (s, 1H), 7.95 − 7.95 (m, 1H), 7.74 (s, 1H), 7.36 − 7.30 (m, 1H), 7.28 − 7.25 (m, 3H), 7.21 − 7.14 (m, 2H), 4.60 (br s, 2H), 2.46 − 2.36 (m, 2H), 1.98 − 1.90 (m, 1H), 1.52 − 1.38 (m, 2H), 0.96 − 0.90 (m, 2H), 0.74 − 0.69 (m, 2H). | |
| Example 46 | 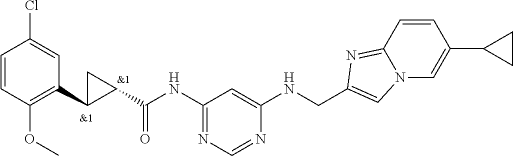 rac-(1S*,2S*)-2-(3-chloro-5- fluorophenyl)-N-(6-(((6- cyclopropylimidazo[1,2-a]pyridin-2- | rac-(1S*,2S*)-2-(3-chloro-5- fluorophenyl)-N-(6-chloropyrimidin- 4-yl)cyclopropane-1-carboxamide ESI-MS (M + H)+: 477.2, ¹H NMR (400 MHz, DMSO) δ 10.59 (s, 1H), 8.31 (s, 1H), 8.21 (s, 1H), 7.87 (br s, 1H), 7.62 (s, 1H), 7.38 (d, J = 9.3 Hz, 1H), 7.29 − 7.23 (m, 2H), 7.17 (s, 1H), 7.09 (d, J = 9.7 Hz, 1H), 6.97 (dd, J = 1.8, 9.3 Hz, 1H), 4.57 (br s, 2H), 2.49 − 2.34 (m, 2H), 1.96 − 1.88 (m, 1H), 1.53 − 1.44 (m, 2H), 0.95 − 0.89 (m, 2H), 0.70 − 0.65 (m, 2H). | |
| | yl)methyl)amino)pyrimidin-4- | | |
| | yl)cyclopropane-1-carboxamide (I-46) | | |
| Example 47 | 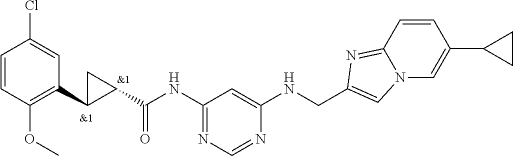 [SMILES: COc1ccc(Cl)cc1[C@H]1C[C@@H]1C(=O)Nc1cc(NCc2cn3cc(C4CC4)ccc3n2)ncn1] rac-(1S*,2S*)-2-(5-chloro-2- methoxyphenyl)-N-(6-(((6- cyclopropylimidazo[1,2-a]pyridin-2- yl)methyl)amino)pyrimidin-4- | rac-(1S*,2S*)-2-(5-chloro-2- methoxyphenyl)-N-(6- chloropyrimidin-4-yl)cyclopropane- 1-carboxamide ESI-MS (M + H)+: 489.4, ¹H NMR (400 MHz, DMSO) δ 10.57 (s, 1H), 8.31 (s, 1H), 8.21 (s, 1H), 7.86 (br s, 1H), 7.62 (s, 1H), 7.38 (d, J = 9.3 Hz, 1H), 7.31 − 7.23 (m, 2H), 7.03 − 6.95 (m, 3H), 4.58 (br s, 2H), 3.81 (s, 3H), 2.53 − 2.47 (m, 1H), 2.35 − 2.28 (m, 1H), 1.96 − 1.88 (m, 1H), 1.44 − 1.34 (m, 2H), 0.95 − 0.89 (m, 2H), 0.70 − 0.65 (m, 2H). | |
| | yl)cyclopropane-1-carboxamide (I-47) | | |
| Example 48 | 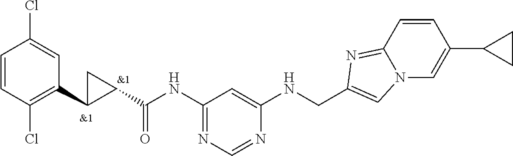 [SMILES: O=C(Nc1cc(NCc2cn3cc(C4CC4)ccc3n2)ncn1)[C@H]1C[C@@H]1c1cc(Cl)ccc1Cl] rac-(1S*,2S*)-2-(5-chloro-2-cyanophenyl)- N-(6-(((6-cyclopropylimidazo[1,2- a]pyridin-2-yl)methyl)amino)pyrimidin-4- yl)cyclopropane-1-carboxamide, formic acid salt (I-48) | rac-(1S*,2S*)-2-(5-chloro-2- cyanophenyl)-N-(6-chloropyrimidin- 4-yl)cyclopropane-1-carboxamide ESI-MS (M + H)+: 484.3, ¹H NMR (400 MHz, DMSO) δ 10.67 − 10.62 (m, 1H), 8.33 − 8.28 (m, 1H), 8.21 (s, 1H), 7.86 (d, J = 8.1 Hz, 2H), 7.61 (s, 1H), 7.52 (dd, J = 1.4, 8.2 Hz, 1H), 7.40 (s, 1H), 7.36 (d, J = 9.3 Hz, 1H), 7.28 (s, 1H), 6.95 (dd, J = 1.0, 9.3 Hz, 1H), 4.57 (br s, 2H), 2.62 − 2.56 (m, 1H), 1.95 − 1.86 (m, 1H), 1.61 − 1.53 (m, 2H), 0.94 − 0.87 (m, 2H), 0.69 − 0.62 (m, 2H). One CH of cyclopropyl group obscured by DMSO peak. | |
| Example 49 | 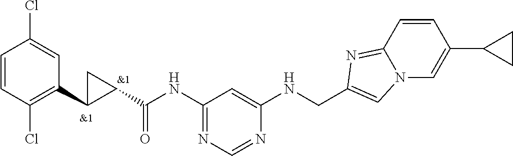 [SMILES: O=C(Nc1cc(NCc2cn3cc(C4CC4)ccc3n2)ncn1)[C@H]1C[C@@H]1c1cc(Cl)ccc1Cl] rac-(1S*,2S*)-N-(6-(((6- cyclopropylimidazo[1,2-a]pyridin-2- yl)methyl)amino)pyrimidin-4-yl)-2-(2,5- dichlorophenyl)cyclopropane-1- | rac-(1S*,2S*)-N-(6-chloropyrimidin- 4-yl)-2-(2,5- dichlorophenyl)cyclopropane-1- carboxamide ESI-MS (M + H)+: 493.4, ¹H NMR (400 MHz, DMSO) δ 10.69 (s, 1H), 8.51 (s, 1H), 8.35 (s, 1H), 8.26 (s, 1H), 7.93 − 7.88 (m, 1H), 7.67 (s, 1H), 7.55 (d, J = 8.6 Hz, 1H), 7.41 − 7.35 (m, 4H), 7.01 (d, J = 9.3 Hz, 1H), 4.63 (br s, 2H), 2.41 − 2.36 (m, 1H), 2.00 − 1.93 (m, 1H), 1.53 (t, J = 6.3 Hz, 2H), 0.99 − 0.94 (m, 2H), 0.74 − 0.68 (m, 2H). | |
| | carboxamide, formic acid salt (I-49) | | |
| Example 50 | 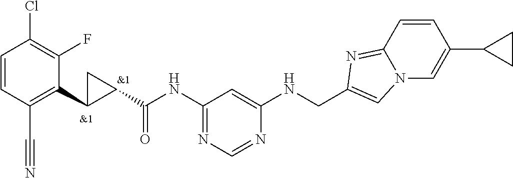 [SMILES: N#Cc1ccc(Cl)c(F)c1[C@H]1C[C@@H]1C(=O)Nc1cc(NCc2cn3cc(C4CC4)ccc3n2)ncn1] rac-(1S*,2S*)-2-(3-chloro-6-cyano-2- fluorophenyl)-N-(6-(((6- cyclopropylimidazo[1,2-a]pyridin-2- yl)methyl)amino)pyrimidin-4- yl)cyclopropane-1-carboxamide, formic acid salt (I-50) | rac- (1S*,2S*)- 2-(3- chloro-6- fluorophenyl)- N-(6- chloropyrimidin- 4-yl) cyclopropane- 1- carboxamide | ESI-MS (M + H)+: 502.3, ¹H NMR (400 MHz, DMSO) δ 10.75 (s, 1H), 8.31 − 8.20 (m, cyano-2- 3H), 7.87 − 7.85 (m, 1H), 7.75 − 7.73 (m, 2H), 7.61 (s, 1H), 7.37 (d, J = 9.3 Hz, 1H), 7.30 − 7.29 (m, 1H), 6.97 − 4.52 (m, 2H), 1.94 − 1.86 (m, 1H), 1.58 − 1.52 (m, 2H), 0.94 − 0.87 (m, 2H), 0.69 − 0.63 (m, 2H). Two CH of cyclopropyl group obscured by DMSO peak. |

| | | |
|---|---|---|
| Example 51 | 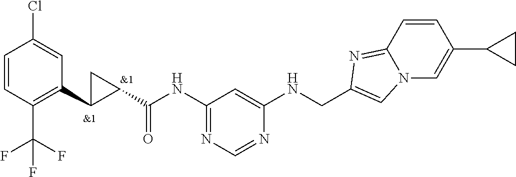 [SMILES: O=C(Nc1cc(NCc2cn3cc(C4CC4)ccc3n2)ncn1)[C@H]1C[C@@H]1c1cc(Cl)ccc1C(F)(F)F] rac-(1S*,2S*)-2-(5-chloro-2- (difluoromethyl)phenyl)-N-(6-(((6- cyclopropylimidazo[1,2-a]pyridin-2- yl)methyl)amino)pyrimidin-4- | rac-(1S*,2S*)-2-(5-chloro-2- (difluoromethyl)phenyl)-N-(6- chloropyrimidin-4-yl)cyclopropane- 1-carboxamide ESI-MS (M + H)+: 509.3, ¹H NMR (400 MHz, DMSO) 8 10.63 − 10.60 (m, 1H), 8.30 − 8.28 (m, 1H), 8.20 (d, J = 0.7 Hz, 1H), 7.86 (br s, 1H), 7.61 (s, 1H), 7.57 (d, J = 8.4 Hz, 1H), 7.45 (q, J = 3.3 Hz, 1H), 7.41 − 7.12 (m, 4H), 6.95 (dd, J = 1.8, 9.3 Hz, 1H), 4.57 (br s, 2H), 2.58 − 2.54 (m, 1H), 2.45 − 2.38 (m, 1H), 1.94 − 1.87 (m, 1H), 1.51 − 1.43 (m, 2H), 0.93 − 0.87 (m, 2H), 0.68 − 0.63 (m, 2H). |
| | yl)cyclopropane-1-carboxamide (I-51) | |
| Example 52 | 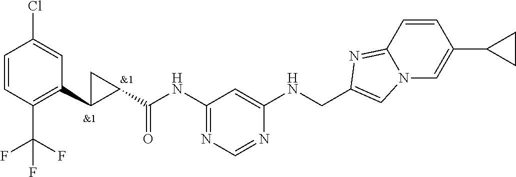 [SMILES: O=C(Nc1cc(NCc2cn3cc(C4CC4)ccc3n2)ncn1)[C@H]1C[C@@H]1c1cc(Cl)ccc1C(F)(F)F] rac-(1S*,2S*)-2-(5-chloro-2- (trifluoromethyl)phenyl)-N-(6-(((6- cyclopropylimidazo[1,2-α]pyridin-2- yl)methyl)amino)pyrimidin-4- | rac-(1S*,2S*)-2-(5-chloro-2- (trifluoromethyl)phenyl)-N-(6- chloropyrimidin-4-yl)cyclopropane- 1-carboxamide ESI-MS (M + H)+: 527.4, ¹H NMR (400 MHz, DMSO) δ 10.68 (s, 1H), 8.35 (s, 1H), 8.26 (s, 1H), 7.92 − 7.89 (m, 1H), 7.79 (d, J = 8.1 Hz, 1H), 7.67 (s, 1H), 7.59 (d, J = 8.3 Hz, 1H), 7.50 (s, 1H), 7.42 (d, J = 9.6 Hz, 1H), 7.34 (s, 1H), 7.01 (d, J = 9.3 Hz, 1H), 4.63 (br s, 2H), 2.49 − 2.46 (m, 1H), 1.99 − 1.92 (m, 1H), 1.71 − 1.66 (m, 1H), 1.56 − 1.50 (m, 1H), 0.99 − 0.94 (m, 2H), 0.74 − 0.68 (m, 2H). |
| | yl)cyclopropane-1-carboxamide (I-52) | |
| Example 53 | 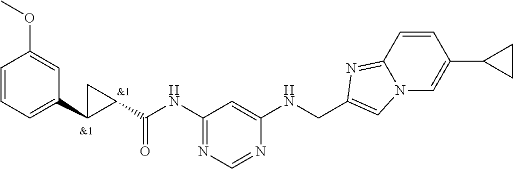 [SMILES: COc1cccc([C@H]2C[C@@H]2C(=O)Nc2cc(NCc3cn4cc(C5CC5)ccc4n3)ncn2)c1] rac-(1S*,2S*)-N-(6- (6- cyclopropylimidazo[1,2-a]pyridin-2- yl)methyl)amino)pyrimidin-4-yl)-2-(3- methoxyphenyl)cyclopropane-1-carboxamide (I-53) | rac-(1S*,2S*)-N-(6-chloropyrimidin- 4-yl)-2-(3- methoxyphenyl)cyclopropane-1- carboxamide ESI-MS (M + H)+: 455.3, ¹H NMR (400 MHz, DMSO) δ 10.64 (s, 1H), 8.35 (s, 1H), 8.24 (s, 1H), 7.89 (s, 1H), 7.66 (s, 1H), 7.42 (d, J = 9.3 Hz, 1H), 7.32 (s, 1H), 7.25 (t, J = 8.1 Hz, 1H), 7.01 (dd, J = 1.5, 9.3 Hz, 1H), 6.84 − 6.75 (m, 3H), 4.59 (m, 2H), 3.80 − 3.78 (m, 3H), 2.44 − 2.37 (m, 2H), 2.00 − 1.92 (m, 1H), 1.53 − 1.36 (m, 2H), 1.00 − 0.93 (m, 2H), 0.74 − 0.68 (m, 2H). |
| Example 54 | 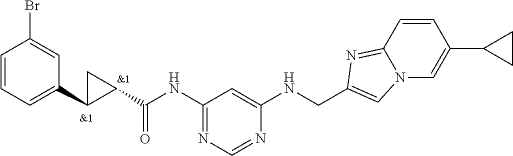 [SMILES: O=C(Nc1cc(NCc2cn3cc(C4CC4)ccc3n2)ncn1)[C@H]1C[C@@H]1c1cccc(Br)c1] rac-(1S*,2S*)-2-(3-bromophenyl)-N-(6- (((6-cyclopropylimidazo[1,2-a]pyridin-2- yl)methyl)amino)pyrimidin-4- yl)cyclopropane-1-carboxamide (I-54) | rac-(1S*,2S*)-2-(3-bromophenyl)-N- (6-chloropyrimidin-4- yl)cyclopropane-1-carboxamide ESI-MS (M + H)+: 503.2, 505.2, ¹H NMR (400 MHz, DMSO) δ 10.60 (s, 1H), 8.32 − 8.30 (m, 1H), 8.23 − 8.20 (m, 1H), 7.91 − 7.82 (m, 1H), 7.62 (s, 1H), 7.42 − 7.37 (m, 3H), 7.30 − 7.24 (m, 2H), 7.22 − 7.18 (m, 1H), 6.99 − 6.95 (m, 1H), 4.62 − 4.52 (m, 2H), 2.45 − 2.35 (m, 2H), 1.95 − 1.89 (m, 1H), 1.52 − 1.45 (m, 1H), 1.44 − 1.38 (m, 1H), 0.95 − 0.89 (m, 2H), 0.70 − 0.65 (m, 2H). |

### Examples 55-71

### Synthesis of rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide (I-55)

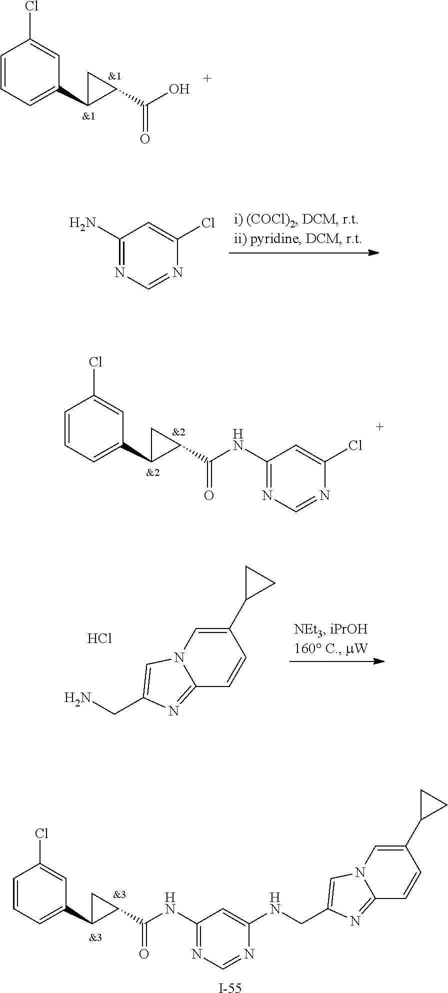

Synthesis of rac-(1S*,2S*)-2-(3-chlorophenyl)-N-(6-chloropyrimidin-4-yl)cyclopropane-1-carboxamide. Oxalyl chloride (0.53 mL, 6.10 mmol) was added to a stirred solution of rac-(1S*,2S*)-2-(3-chlorophenyl)cyclopropane-1-carboxylic acid (0.40 g, 2.0 mmol) and DMF (2 drops) in DCM (8.0 mL). The mixture was stirred at room temperature for 1 h then concentrated in vacuo. The residue was re-dissolved in DCM (20 mL), and 6-chloropyrimidin-4-amine (0.27 g, 2.1 mmol) and pyridine (0.25 mL, 3.1 mmol) were added. The mixture was stirred at room temperature under a nitrogen atmosphere for 18 h. The mixture was diluted with DCM (100 mL), washed with water (50 mL), dried over MgSO₄, filtered, and concentrated in vacuo to give the title compound (0.61 g, 96%) as a yellow solid. ESI-MS (M+H)+: 308.1, ¹H NMR (400 MHz, DMSO) δ 11.57 (s, 1H), 8.80 (s, 1H), 8.19 (s, 1H), 7.39 (m, 1H), 7.35-7.30 (m, 2H), 7.23 (d, J=7.3 Hz, 1H), 2.49-2.44 (m, 1H), 1.64-1.54 (m, 2H), 1H hidden under solvent peaks.

Synthesis of rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide (I-55). NEt₃ (0.063 mL, 0.13 mmol) was added to a suspension of rac-(1S*,2S*)-2-(3-chlorophenyl)-N-(6-chloropyrimidin-4-yl)cyclopropane-1-carboxamide (0.040 g, 0.13 mmol) and (6-cyclopropylimidazo[1,2-a]pyridin-2-yl)methanamine hydrochloride (0.029 g, 0.13 mmol) in iPrOH (1.0 mL). The mixture was stirred at 160° C. in a microwave reactor for 1.5 h. The mixture was concentrated in vacuo. The residue was re-dissolved in DCM and washed with water. The aqueous layer was extracted with DCM (2×) and the combined organic layers were passed through a hydrophobic frit and concentrated in vacuo. The residue was purified by reverse phase preparative HPLC to give the title compound (0.036 g, 61%) as a beige solid (mixture of enantiomers). ESI-MS (M+H): 459.3, ¹H NMR (400 MHz, DMSO) δ 10.60 (s, 1H), 8.29 (d, J=9.8 Hz, 1H), 8.21 (s, 1H), 7.86 (br s, 1H), 7.62 (s, 1H), 7.40-7.30 (m, 2H), 7.28-7.25 (m, 3H), 7.16 (d, J=7.7 Hz, 1H), 6.97 (dd, J=1.8, 9.3 Hz, 1H), 4.57 (br s, 2H), 2.45-2.34 (m, 2H), 1.96-1.88 (m, 1H), 1.52-1.38 (m, 2H), 0.95-0.89 (m, 2H), 0.70-0.65 (m, 2H).

The mixture was separated using SFC (LUX Cellulose-4 20×250 mm, 5 μm 55/45 MeOH (0.1% DEA)/CO₂, 70 mL/min, 120 bar, 40° C.) to give two enantiomers.

First eluting isomer, (1R,2R)-2-(3-chlorophenyl)-N-(6-(((6-cyclopropylimidazo[1,2-a]pyridin-2-yl)methyl)amino)pyrimidin-4-yl)cyclopropane-1-carboxamide (I-56): ESI-MS (M+H): 459.3, ¹H NMR (400 MHz, DMSO) δ 10.58 (s, 1H), 8.29 (s, 1H), 8.19 (s, 1H), 7.85-7.84 (m, 1H), 7.60 (s, 1H), 7.39-7.28 (m, 2H), 7.26 (d, J=5.1 Hz, 3H), 7.15 (d, J=7.6 Hz, 1H), 6.98-6.94 (m, 1H), 4.56-4.55 (m, 2H), 2.41-2.35 (m, 2H), 1.95-1.86 (m, 1H), 1.50-1.36 (m, 2H), 0.94-0.87 (m, 2H), 0.69-0.62 (m, 2H). RT=1.44 min, 99.3% e.e.

Second eluting isomer, (1S,2S)-2-(3-chlorophenyl)-N-(6-(((6-cyclopropylimidazo[1,2-a]pyridin-2-yl)methyl)amino)pyrimidin-4-yl)cyclopropane-1-carboxamide (I-57): ESI-MS (M+H): 459.3 ¹H NMR (400 MHz, DMSO) δ 10.60 (s, 1H), 8.31 (s, 1H), 8.21 (s, 1H), 7.86 (br s, 1H), 7.62 (s, 1H), 7.40-7.30 (m, 2H), 7.28-7.25 (m, 3H), 7.16 (d, J=7.7 Hz, 1H), 6.97 (dd, J=1.7, 9.3 Hz, 1H), 4.57 (br s, 2H), 2.45-2.36 (m, 2H), 1.96-1.88 (m, 1H), 1.51-1.38 (m, 2H), 0.95-0.89 (m, 2H), 0.70-0.65 (m, 2H). RT=3.93 min, 98.2% e.e.

Using a similar procedure to that used for rac-(1S*,2S*)-2-(3-chlorophenyl)-N-(6(((6-cyclopropylimidazo[N,2-a]pyridin-2-yl)methyl)amino)pyrimidin-4-yl)cyclopropane-1-carboxamide, the compounds in Table 9 were prepared from rac-(1S*,2S*)-2-(3-chlorophenyl)-N-(6-chloropyrimidin-4-yl)cyclopropane-1-carboxamide and an appropriate coupling partner.

TABLE 9

| | | Coupling Partner/ |
|---|---|---|
| Example | Compound | Analytical Data |
| Example 58 | 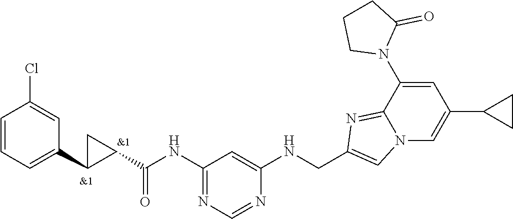 [SMILES: O=C(Nc1cc(NCc2cn3cc(C4CC4)cc(N4CCCC4=O)c3n2)ncn1)[C@H]1C[C@@H]1c1cccc(Cl)c1] | 1-(2-(aminomethyl)-6- cyclopropylimidazo[1,2-a]pyridin-8- yl)pyrrolidin-2-one ESI-MS (M + H): 542.3, ¹H NMR (400 MHz, DMSO) δ 10.62 (s, 1H), 8.26 (s, 1H), 8.22 (s, 1H), 7.90 (s, 1H), 7.67 (s, 1H), 7.36 − 7.24 (m, 4H), 7.18 (d, J = 7.6 Hz, 1H), 7.14 (s, 1H), 4.58 (br s, 2H), 4.17 (m, 2H), 2.51 − 2.39 (m, 3H), 2.20 − 2.09 (m, 2H), 1.98 − 1.90 (m, 1H), 1.52 − 1.39 (m, 2H), 0.98 − 0.91 (m, 2H), 0.67 (m, 2H). |
| | rac-(1S*,2S*)-2-(3-chlorophenyl)-N-(6- | |
| | (((6-cyclopropyl-8-(2-oxopyrrolidin-1- | |
| | yl)imidazo[1,2-α]pyridin-2- | |
| | yl)methyl)amino)pyrimidin-4- | |
| | yl)cyclopropane-1-carboxamide (I-58) | |
| Example 59 | 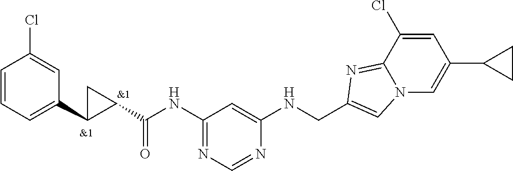 [SMILES: O=C(Nc1cc(NCc2cn3cc(C4CC4)cc(Cl)c3n2)ncn1)[C@H]1C[C@@H]1c1cccc(Cl)c1] | (8-chloro-6-cyclopropylimidazo[1,2- a]pyridin-2-yl)methanamine ESI-MS (M + H): 493.3, ¹H NMR (400 MHz, DMSO) δ 10.61 (s, 1H), 8.34 (s, 1H), 8.21 (s, 1H), 7.95 (m, 1H), 7.74 (s, 1H), 7.36 − 7.30 (m, 1H), 7.28 − 7.25 (m, 3H), 7.21 − 7.14 (m, 2H), 4.60 (br s, 2H), 2.46 − 2.36 (m, 2H), 1.98 − 1.90 (m, 1H), 1.52 − 1.38 (m, 2H), 0.96 − 0.90 (m, 2H), 0.74 − 0.69 (m, 2H). |
| | rac-(1S*,2S*)-N-(6-(((8-chloro-6- | |
| | cyclopropylimidazo[1,2-α]pyridin-2- | |
| | yl)methyl)amino)pyrimidin-4-yl)-2-(3- | |
| | chlorophenyl)cyclopropane-1- | |
| | carboxamide (I-59) | |
| Example 60 | 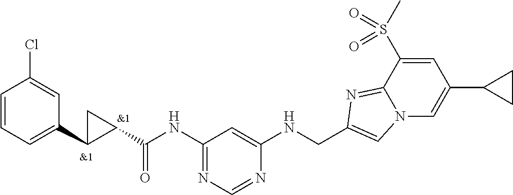 [SMILES: CS(=O)(=O)c1cc(C2CC2)cn2cc(CNc3cc(NC(=O)[C@H]4C[C@@H]4c4cccc(Cl)c4)ncn3)nc12] | (6-cyclopropyl-8- (methylsulfonyl)imidazo[1,2- a]pyridin-2-yl)methanamine ESI-MS (M + H): 537.2, ¹H NMR (400 MHz, DMSO) 10.66 (s, 1H), 8.68 (s, 1H), 8.25 (s, 1H), 8.03 (s, 1H), 7.86 (s, 1H), 7.62 (s, 1H), 7.40 − 7.18 (m, 5H), 4.70 (s, 2H), 3.59 (s, 3H), 2.48 − 2.40 (m, 2H), 2.16 − 2.08 (m, 1H), 1.56 − 1.43 (m, 2H), 1.06 − 0.99 (m, 2H), 0.79 − 0.73 (m, 2H). |
| | rac-(1S*,2S*)-2-(3-chlorophenyl)-N-(6- | |
| | (((6-cyclopropyl-8- | |
| | (methylsulfonyl)imidazo[1,2-α]pyridin- | |
| | 2-yl)methyl)amino)pyrimidin-4- | |
| | yl)cyclopropane-1-carboxamide (I-60) | |
| Example 61 | 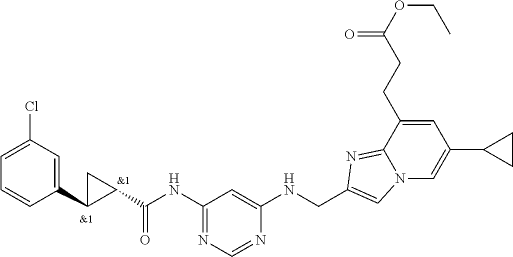 [SMILES: CCOC(=O)CCc1cc(C2CC2)cn2cc(CNc3cc(NC(=O)[C@H]4C[C@@H]4c4cccc(Cl)c4)ncn3)nc12] | ethyl 3-(2-(aminomethyl)-6- cyclopropylimidazo[1,2-a]pyridin-8- yl)propanoate ESI-MS (M + H): 559.4, ¹H NMR (400 MHz, DMSO) δ 10.60 (s, 1H), 8.18 (d, J = 8.3 Hz, 2H), 7.90 (br s, 1H), 7.58 (s, 1H), 7.34 − 7.24 (m, 4H), 7.15 (d, J-6.8 Hz, 1H), 6.78 (s, 1H), 4.57 (br s, 2H), 4.06 (q, J = 7.1 Hz, 2H), 3.10 − 3.05 (m, 2H), 2.83 − 2.77 (m, 2H), 2.43 − 2.35 (m, 2H), 1.89 − 1.82 (m, 1H), 1.48 − 1.37 (m, 2H), 1.16 (t, J = 7.1 Hz, 3H), 0.91 − 0.86 (m, 2H), 0.66 − 0.61 (m, 2H). |
| | rac-ethyl 3-(2-(((6-((1S*,2S*)-2-(3- | |
| | chlorophenyl)cyclopropane-1- | |
| | carboxamido)pyrimidin-4- | |
| | yl)amino)methyl)-6- | |
| | cyclopropylimidazo[1,2-α]pyridin-8- | |
| | yl)propanoate (I-61) | |
| Example 62 | 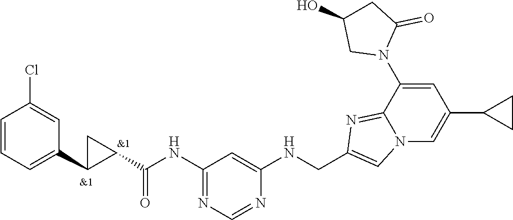 [SMILES: O=C(Nc1cc(NCc2cn3cc(C4CC4)cc(N4C[C@@H](O)CC4=O)c3n2)ncn1)[C@H]1C[C@@H]1c1cccc(Cl)c1] | (S)-1-(2-(aminomethyl)-6- cyclopropylimidazo[1,2-a]pyridin-8- yl)-4-hydroxypyrrolidin-2-one ESI-MS (M + H): 558.4, ¹H NMR (400 MHz, DMSO) 10.61 (s, 1H), 8.23 (d, J = 1.4 Hz, 1H), 8.21 (s, 1H), 7.90 (s, 1H), 7.66 (s, 1H), 7.35 − 7.15 (m, 6H), 5.37 (s, 1H), 4.58 (s, 2H), 4.49 − 4.43 (m, 2H), 4.00 − 3.96 (m, 1H), 2.86 − 2.78 (m, 1H), 2.45 − 2.37 (m, 2H), 2.34 − 2.28 (m, 1H), 1.97 − 1.90 (m, 1H), 1.49 (t, J = 9.0 Hz, 1H), 1.45 − 1.38 (m, 1H), 0.96 − 0.91 (m, 2H), 0.68 − 0.62 (m, 2H) |
| | (rac-(1S*,2S*))-2-(3-chlorophenyl)-N- | |
| | (6-(((6-cyclopropyl-8-((S)-4-hydroxy-2- | |
| | oxopyrrolidin-1-yl)imidazo[1,2- | |
| | a]pyridin-2-yl)methyl)amino)pyrimidin- | |
| | 4-yl)cyclopropane-1-carboxamide (I-62) | |
| Example 63 | 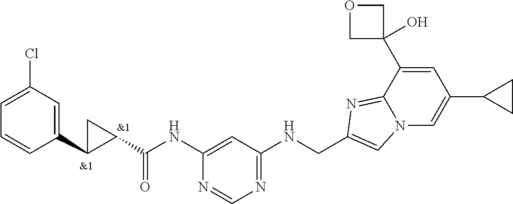 [SMILES: O=C(Nc1cc(NCc2cn3cc(C4CC4)cc(C4(O)COC4)c3n2)ncn1)[C@H]1C[C@@H]1c1cccc(Cl)c1] rac-(1S*,2S*)-2-(3-chlorophenyl)-N-(6- | (6-cyclopropyl-8-(3-hydroxyoxetan- 3-yl)imidazo[1,2-a]pyridin-2- yl)methanamine ESI-MS (M + H): 3.3, ¹H NMR (400 MHz, DMSO) δ 10.62 (s, 1H), 8.28 (d, J = 1.6 Hz, 1H), 8.21 (s, 1H), 7.89 (s, 1H), 7.65 (s, 1H), 7.36 − 7.25 (m, 4H), 7.16 (d, J = 8.0 Hz, 1H), 7.05 (d, J = 1.8 Hz, 1H), 6.49 (s, 1H), 5.25 (d, J = 6.6 Hz, 2H), 4.68 (d, J-6.6 Hz, 2H), 4.59 (s, 2H), 2.45 − 2.35 (m, 2H), 1.99 − 1.91 (m, 1H), 1.52 − 1.45 (m, 1H), 1.45 − 1.39 (m, 1H), 0.96 − 0.90 (m, 2H), 0.72 − 0.67 (m, 2H). |
| | (((6-cyclopropyl-8-(3-hydroxyoxetan-3- | |
| | yl)imidazo[1,2-α]pyridin-2- | |
| | yl)methyl)amino)pyrimidin-4- | |
| | yl)cyclopropane-1-carboxamide (I-63) | |
| Example 64 | 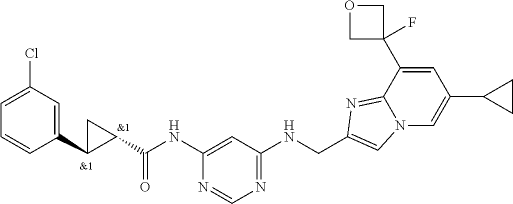 [SMILES: O=C(Nc1cc(NCc2cn3cc(C4CC4)cc(C4(F)COC4)c3n2)ncn1)[C@H]1C[C@@H]1c1cccc(Cl)c1] | (6-cyclopropyl-8-(3-fluorooxetan-3- yl)imidazo[1,2-a]pyridin-2- yl)methanamine ESI-MS (M + H): 533.4, ¹H NMR (400 MHz, DMSO) 8 10.65 (s, 1H), 8.44 (s, 1H), 8.25 (s, 1H), 7.93 (br s, 1H), 7.73 (s, 1H), 7.38 − 7.29 (m, 4H), 7.23 − 7.15 (m, 2H), 5.40 (dd, J = 8.8, 26.3 Hz, 2H), 5.03 (dd, J = 8.7, 22.4 Hz, 2H), 4.63 (br s, 2H), 2.45 − 2.38 (m, 2H), 2.04 − 1.96 (m, 1H), 1.54 − 1.43 (m, 2H), 0.98 (m, 2H), 0.80 − 0.74 (m, 2H). |
| | rac-(1S*,2S*)-2-(3-chlorophenyl)-N-(6- | |
| | (((6-cyclopropyl-8-(3-fluorooxetan-3- | |
| | yl)imidazo[1,2-α]pyridin-2- | |
| | yl)methyl)amino)pyrimidin-4- | |
| | yl)cyclopropane-1-carboxamide (I-64) | |
| Example 65 | 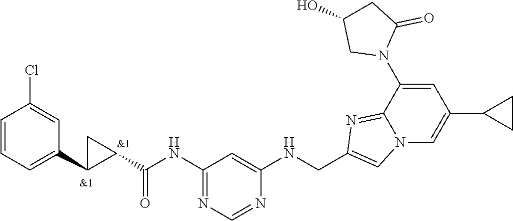 [SMILES: O=C(Nc1cc(NCc2cn3cc(C4CC4)cc(N4C[C@H](O)CC4=O)c3n2)ncn1)[C@H]1C[C@@H]1c1cccc(Cl)c1] | (R)-1-(2-(aminomethyl)-6- cyclopropylimidazo[1,2-a]pyridin-8- yl)-4-hydroxypyrrolidin-2-one ESI-MS (M + H): 558.4, ¹H NMR (400 MHz, DMSO) δ 10.65 (s, 1H), 8.27 (d, J = 7.9 Hz, 2H), 7.94 (s, 1H), 7.70 (s, 1H), 7.38 − 7.20 (m, 6H), 5.40 (d, J = 2.5 Hz, 1H), 4.62 (s, 2H), 4.52 − 4.47 (m, 2H), 4.01 (d, J = 8.4 Hz, 1H), 2.90 − 2.83 (m, 1H), 2.48 − 2.33 (m, 3H), 2.00 − 1.94 (m, 1H), 1.55 − 1.43 (m, 2H), 1.01 − 0.95 (m, 2H), 0.72 − 0.67 (m, 2H). |
| | rac-(1S*,2S*)-2-(3-chlorophenyl)-N-(6- | |
| | (((6-cyclopropyl-8-((R)-4-hydroxy-2- | |
| | oxopyrrolidin-1-yl)imidazo[1,2- | |
| | a]pyridin-2-yl)methyl)amino)pyrimidin- | |
| | 4-yl)cyclopropane-1-carboxamide (I-65) | |
| Example 66 | 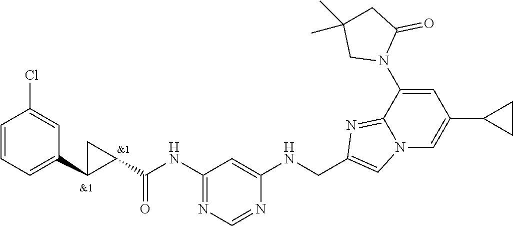 [SMILES: CC1(C)CC(=O)N(c2cc(C3CC3)cn3cc(CNc4cc(NC(=O)[C@H]5C[C@@H]5c5cccc(Cl)c5)ncn4)nc23)C1] | 1-(2-(aminomethyl)-6- cyclopropylimidazo[1,2-a]pyridin-8- yl)-4,4-dimethylpyrrolidin-2-one ESI-MS (M + H): 570.5, ¹H NMR (400 MHz, DMSO) δ 10.61 (s, 1H), 8.26 − 8.20 (m, 2H), 7.88 (s, 1H), 7.65 (s, 1H), 7.36 − 7.26 (m, 4H), 7.17 − 7.14 (m, 2H), 4.57 (s, 2H), 3.90 (s, 2H), 2.45 − 2.37 (m, 2H), 2.34 (s, 2H), 1.96 − 1.89 (m, 1H), 1.51 − 1.37 (m, 2H), 1.22 (s, 6H), 0.96 − 0.90 (m, 2H), 0.68 − 0.63 (m, 2H). |
| | rac-(1S*,2S*)-2-(3-chlorophenyl)-N-(6- | |
| | (((6-cyclopropyl-8-(4,4-dimethyl-2- | |
| | oxopyrrolidin-1-yl)imidazo[1,2- | |
| | a]pyridin-2-yl)methyl)amino)pyrimidin- | |
| | 4-yl)cyclopropane-1-carboxamide (I-66) | |
| Example 67 | 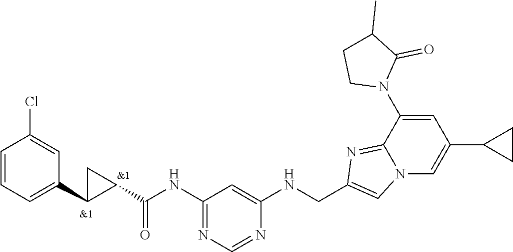 [SMILES: CC1CCN(c2cc(C3CC3)cn3cc(CNc4cc(NC(=O)[C@H]5C[C@@H]5c5cccc(Cl)c5)ncn4)nc23)C1=O] | 1-(2-(aminomethyl)-6- cyclopropylimidazo[1,2-a]pyridin-8- yl)-3-methylpyrrolidin-2-one ESI-MS (M + H): 556.3, ¹H NMR (400 MHz, DMSO) δ 10.59 (s, 1H), 8.23 (s, 1H), 8.19 (s, 1H), 7.64 (s, 1H), 7.34 − 7.24 (m, 4H), 7.15 (d, J = 8.3 Hz, 2H), 4.55 (br s, 2H), 4.17 − 4.01 (m, 2H), 2.68 − 2.57 (m, 1H), 2.44 − 2.31 (m, 3H), 1.95 − 1.87 (m, 1H), 1.80 − 1.68 (m, 1H), 1.50 − 1.37 (m, 2H), 1.18 (d, J = 7.1 Hz, 3H), 0.95 − 0.89 (m, 2H), 0.66 − 0.61 (m, 2H). |
| | rac-(1S*,2S*)-2-(3-chlorophenyl)-N-(6- | |
| | (((6-cyclopropyl-8-(3-methyl-2- | |
| | oxopyrrolidin-1-yl)imidazo[1,2- | |
| | a]pyridin-2-yl)methyl)amino)pyrimidin- | |
| | 4-yl)cyclopropane-1-carboxamide (I-67) | |
| Example 68 | 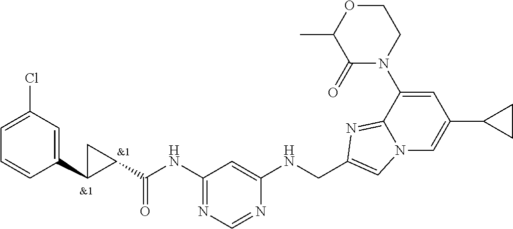 [SMILES: CC1OCCN(c2cc(C3CC3)cn3cc(CNc4cc(NC(=O)[C@H]5C[C@@H]5c5cccc(Cl)c5)ncn4)nc23)C1=O] rac-(1S*,2S*)-2-(3-chlorophenyl)-N-(6- (((6-cyclopropyl-8-(2-methyl-3- | 4-(2-(aminomethyl)-6- cyclopropylimidazo[1,2-a]pyridin-8- yl)-2-methylmorpholin-3-one ESI-MS (M + H): 572.4, ¹H NMR (400 MHz, DMSO) δ 10.60 (s, 1H), 8.40 (s, 0.2H), 8.31 − 8.29 (m, 1H), 8.20 − 8.18 (m, 1H), 7.90 (br s, 1H), 7.65 (s, 1H), 7.34 − 7.24 (m, 4H), 7.17 − 7.13 (m, 1H), 6.99 (d, J = 1.5 Hz, 1H), 4.56 (br s, 2H), 4.36 (q, J = 6.8 Hz, 1H), 4.10 − 4.05 (m, 1H), 3.99 − 3.95 (m, 2H), 3.74 − 3.72 (m, 1H), 2.43 − 2.34 (m, 2H), 1.96 − 1.88 (m, 1H), 1.49 − 1.44 (m, 1H), 1.41 − 1.38 (m, 4H), 0.95 − 0.89 (m, 2H), 0.69 − 0.64 (m, 2H). |
| | oxomorpholino)imidazo[1,2-α]pyridin-2- | |
| | yl)methyl)amino)pyrimidin-4- | |
| | yl)cyclopropane-1-carboxamide, formic | |
| | acid salt (I-68) | |
| Example 69 | 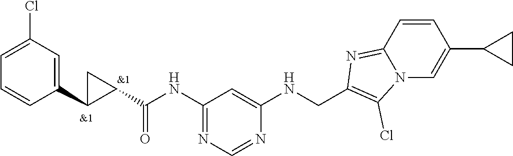 [SMILES: O=C(Nc1cc(NCc2nc3ccc(C4CC4)cn3c2Cl)ncn1)[C@H]1C[C@@H]1c1cccc(Cl)c1] rac-(1S*,2S*)-N-(6-(((3-chloro-6- cyclopropylimidazo[1,2-α]pyridin-2- | (3-chloro-6-cyclopropylimidazo[1,2- a]pyridin-2-yl)methanamine ESI-MS (M + H): 493.3, ¹H NMR (400 MHz, DMSO) 8 10.51 (s, 1H), 8.14 (s, 1H), 8.03 − 8.03 (m, 1H), 7.77 (s, 1H), 7.43 (d, J = 9.3 Hz, 1H), 7.26 − 7.18 (m, 4H), 7.09 (td, J = 1.3, 7.6 Hz, 1H), 6.98 (dd, J = 1.7, 9.4 Hz, 1H), 4.55 (s, 2H), 2.38 − 2.28 (m, 2H), 2.06 − 1.98 (m, 1H), 1.44 − 1.30 (m, 2H), 0.93 − 0.87 (m, 2H), 0.72 − 0.67 (m, 2H). |
| | yl)methyl)amino)pyrimidin-4-yl)-2-(3- | |
| | chlorophenyl)cyclopropane-1- | |
| | carboxamide (I-69) | |
| Example 70 | 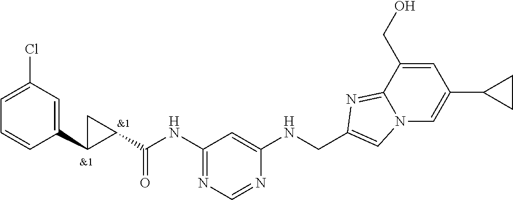 [SMILES: O=C(Nc1cc(NCc2cn3cc(C4CC4)cc(CO)c3n2)ncn1)[C@H]1C[C@@H]1c1cccc(Cl)c1] rac-(1S*,2S*)-2-(3-chlorophenyl)-N-(6- | (2-(aminomethyl)-6- cyclopropylimidazo[1,2-a]pyridin-8- yl)methanol hydrochloride ESI-MS (M + H): 489.3, ¹H NMR (400 MHz, DMSO) δ 10.59 (s, 1H), 8.44 (s, 0.4H), 8.19 − 8.18 (m, 2H), 7.86 (br s, 1H), 7.60 (s, 1H), 7.34 − 7.29 (m, 1H), 7.26 − 7.24 (m, 3H), 7.17 − 7.13 (m, 1H), 6.97 − 6.96 (m, 1H), 5.31 (br s, 1H), 4.77 (s, 2H), 4.56 − 4.55 (m, 2H), 2.44 − 2.32 (m, 2H), 1.96 − 1.88 (m, 1H), 1.50 − 1.43 (m, 1H), 1.43 − 1.37 (m, 1H), 0.94 − 0.88 (m, 2H), 0.67 − 0.62 (m, 2H). |
| | (((6-cyclopropyl-8- | |
| | (hydroxymethyl)imidazo[1,2-α]pyridin- | |
| | 2-yl)methyl)amino)pyrimidin-4- | |
| | yl)cyclopropane-1-carboxamide, formic | |
| | acid salt (I-70) | |
| Example 71 | 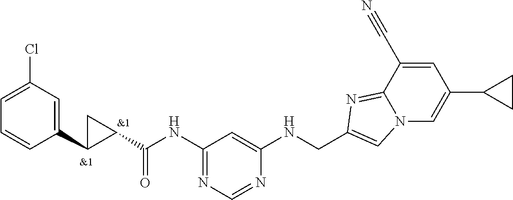 [SMILES: N#Cc1cc(C2CC2)cn2cc(CNc3cc(NC(=O)[C@H]4C[C@@H]4c4cccc(Cl)c4)ncn3)nc12] | 2-(aminomethyl)-6- cyclopropylimidazo[1,2-a]pyridine- 8-carbonitrile ESI-MS (M + H): 484.3, ¹H NMR (400 MHz, DMSO) δ 10.66 (s, 1H), 8.70 (s, 1H), 8.25 (s, 1H), 8.06 − 8.00 (m, 1H), 7.86 − 7.77 (m, 2H), 7.39 − 7.29 (m, 4H), 7.20 (d, J = 7.3 Hz, 1H), 4.67 − 4.67 (m, 2H), 2.47 − 2.38 (m, 2H), 2.05 − 1.98 (m, 1H), 1.55 − 1.42 (m, 2H), 1.03 − 0.96 (m, 2H), 0.83 − 0.77 (m, 2H). |
| | rac-(1S*,2S*)-2-(3-chlorophenyl)-N-(6- | |
| | (((8-cyano-6-cyclopropylimidazo[1,2- | |
| | a]pyridin-2-yl)methyl)amino)pyrimidin- | |
| | 4-yl)cyclopropane-1-carboxamide (I-71) | |

### Example 72

### Synthesis of rac-3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoic acid (I-72)

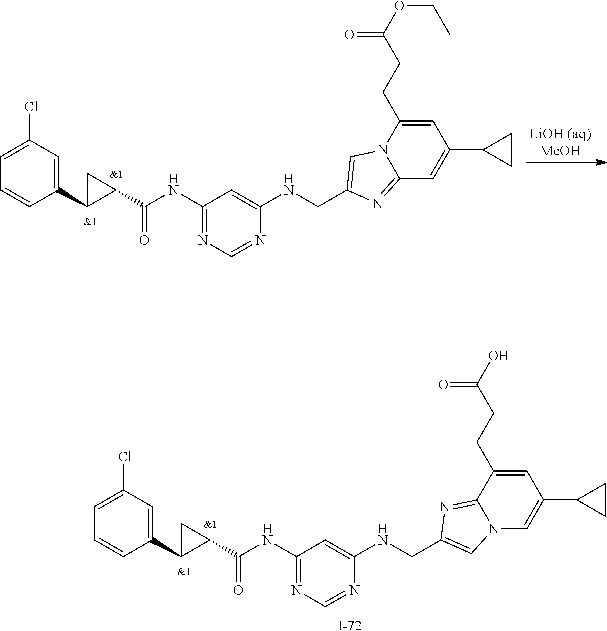

LiOH (1M, aq·, 0.070 mL) was added to a solution of rac-ethyl 3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoate (26 mg, 0.046 mmol) in MeOH (1.0 mL). The mixture was stirred for 18 h, then LiOH (1M, aq·, 0.070 mL) was added. The mixture was stirred at room temperature for a further 3 h, then at 50° C. for 3 h. The mixture was cooled to room temperature, then water (25 mL) was added. The pH was adjusted to ca. 6. The mixture was extracted with DCM (25 mL), then EtOAc (2×25 mL). The combined organics were dried over MgSO₄, then concentrated in vacuo. The residue was purified by preparative HPLC to give the title compound (4.6 mg, 18%). ESI-MS (M+H)+: 531.3, ¹H NMR (400 MHz, DMSO) δ 10.58 (s, 1H), 8.20-8.19 (m, 1H), 8.15-8.13 (m, 1H), 7.92-7.86 (m, 1H), 7.57 (s, 1H), 7.34-7.24 (m, 4H), 7.17-7.13 (m, 1H), 6.79-6.78 (m, 1H), 4.56 (br s, 2H), 3.04 (t, J=7.7 Hz, 2H), 2.66-2.61 (m, 2H), 2.44-2.32 (m, 2H), 1.90-1.82 (m, 1H), 1.50-1.36 (m, 2H), 0.91-0.85 (m, 2H), 0.66-0.61 (m, 2H).

### Example 73

### Synthesis of rac-(1S*,2S*)-2-(3-chlorophenyl)-N-(6-(((6-cyclopropyl-8-(morpholinomethyl)imidazo[1,2-a]pyridin-2-yl)methyl)amino)pyrimidin-4-yl)cyclopropane-1-carboxamide (I-73)

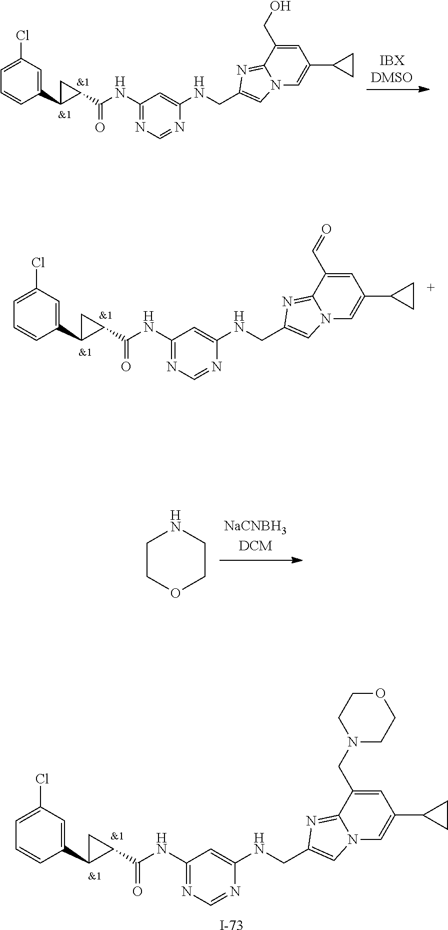

IBX (45%, 84 mg, 0.135 mmol) was added to a solution of rac-(1S*,2S*)-2-(3-chlorophenyl)-N-(6-(((6-cyclopropyl-8-(hydroxymethyl)imidazo[1,2-a]pyridin-2-yl)methyl)amino)pyrimidin-4-yl)cyclopropane-1-carboxamide (55 mg, 0.11 mmol) in DMSO (1.0 mL). The mixture was stirred for 3 h, then Na₂S₂O₃ (sat·aq·, 5 mL) was added. The mixture was stirred for 5 min, then NaHCO₃ (sat·aq·, 5 mL) was added. The mixture was stirred for a further 5 min, then diluted with NaHCO₃ (sat·aq·, 25 mL), then extracted with DCM (2×25 mL). The combined organics were washed with brine (25 mL), passed through a phase separator cartridge, then concentrated in vacuo. The residue (55 mg) was dissolved in DCM (5.0 mL), then morpholine (0.020 mL, 0.23 mmol) was added. After stirring for 30 min, NaCNBH₃ (14 mg, 0.23 mmol) was added and the mixture was stirred for 18 h. Further morpholine (0.020 mL, 0.23 mmol) was added, and after a further 2 h stirring, NaCNBH₃ (14 mg, 0.23 mmol) then MeOH (0.1 mL) were added. The mixture was stirred for 2 h then morpholine (0.020 mL, 0.23 mmol) was added. The mixture was stirred for 18 h, then diluted with NaHCO₃ (sat·aq·, 15 mL). The mixture was extracted with DCM (25 mL) the EtOAc (2×25 mL). The combined organics were washed with brine, passed through a phase separator column then concentrated in vacuo. The residue was purified by preparative HPLC to give the title compound as a formic acid salt. ESI-MS (M+H)+: 558.4, ¹H NMR (400 MHz, DMSO) δ 10.59 (s, 1H), 8.19 (d, J=0.7 Hz, 2H), 7.87 (br s, 1H), 7.60 (s, 1H), 7.34-7.24 (m, 4H), 7.17-7.13 (m, 1H), 6.96 (d, J=1.3 Hz, 1H), 4.56 (s, 2H), 3.76 (s, 2H), 3.61 (t, J=4.5 Hz, 4H), 2.48-2.31 (m, 6H), 1.96-1.88 (m, 1H), 1.49-1.37 (m, 2H), 0.94-0.88 (m, 2H), 0.66-0.61 (m, 2H).

### Examples 74-76

### Synthesis of rac-(1S*,2S*)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide (I-74)

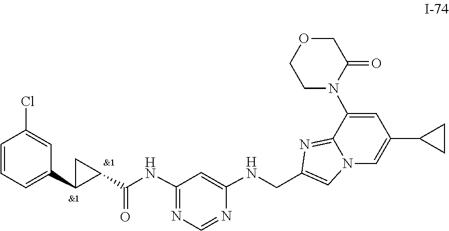 [SMILES: O=C(Nc1cc(NCc2cn3cc(C4CC4)cc(N4CCOCC4=O)c3n2)ncn1)[C@H]1C[C@@H]1c1cccc(Cl)c1]

Using a similar procedure to that used for rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide, the title compound was prepared using rac-(1S*,2S*)-2-(3-chlorophenyl)-N-(6-chloropyrimidin-4-yl)cyclopropane-1-carboxamide (32 mg, 0.11 mmol) and 4-(2-(aminomethyl)-6-cyclopropylimidazo[1,2-a]pyridin-8-yl)morpholin-3-one (30 mg, 0.11 mmol), then purified by preparative LCMS to give the title compound (23 mg, 39%) as a mixture of enantiomers. ESI-MS (M+H)+: 558.4, ¹H NMR (400 MHz, DMSO) δ 10.65 (s, 1H), 8.37 (s, 1H), 8.25 (s, 1H), 7.96 (br s, 1H), 7.72 (s, 1H), 7.38-7.28 (m, 4H), 7.21 (d, J=7.6 Hz, 1H), 7.07 (s, 1H), 4.66-4.57 (m, 2H), 4.31 (s, 2H), 4.08 (m, 2H), 3.90 (m, 2H), 2.47-2.39 (m, 2H), 2.02-1.94 (m, 1H), 1.55-1.42 (m, 2H), 1.01-0.95 (m, 2H), 0.75-0.69 (m, 2H).

The mixture was separated using SFC (YMC Amylose-C 10×250 mm, 5 μm 55/45 EtOH (0.1% DEA)/CO₂, 15 mL/min, 120 bar, 40° C.) to give two enantiomers: (1S,2S)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide and (1R,2R)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide.

First eluting isomer (I-75, 4.7 mg): ESI-MS (M+H): 558.4, ¹H NMR (400 MHz, DMSO) 10.61 (s, 1H), 8.33 (s, 1H), 8.21 (s, 1H), 7.94 (s, 1H), 7.68 (s, 1H), 7.36-7.27 (m, 4H), 7.17 (d, J=7.7 Hz, 1H), 7.03 (d, J=1.0 Hz, 1H), 4.58-4.58 (m, 2H), 4.27 (s, 2H), 4.06-4.01 (m, 2H), 3.88-3.84 (m, 2H), 2.43-2.34 (m, 2H), 1.98-1.90 (m, 1H), 1.51-1.39 (m, 2H), 0.97-0.91 (m, 2H), 0.71-0.65 (m, 2H). RT=2.5 min, 99.9% e.e.

Second eluting isomer (I-76, 3.8 mg): ESI-MS (M+H): 558.4, ¹H NMR (400 MHz, DMSO) 10.61 (s, 1H), 8.33 (d, J=1.1 Hz, 1H), 8.21 (s, 1H), 7.93 (s, 1H), 7.68 (s, 1H), 7.36-7.26 (m, 4H), 7.16 (d, J=7.7 Hz, 1H), 7.03 (d, J=1.5 Hz, 1H), 4.58 (s, 2H), 4.27 (s, 2H), 4.06-4.01 (m, 2H), 3.88-3.84 (m, 2H), 2.43-2.33 (m, 2H), 1.98-1.90 (m, 1H), 1.52-1.39 (m, 2H), 0.97-0.91 (m, 2H), 0.71-0.66 (m, 2H). RT=5.1 min, 99.9% e.e.

### Example 77

### Synthesis of (1S,2S)-2-(3-chlorophenyl)-N-(6-(((6-cyclopropylimidazo[1,2-a]pyridin-2-yl)methyl)(2-hydroxyethyl)amino)pyrimidin-4-yl)cyclopropane-1-carboxamide (I-77)

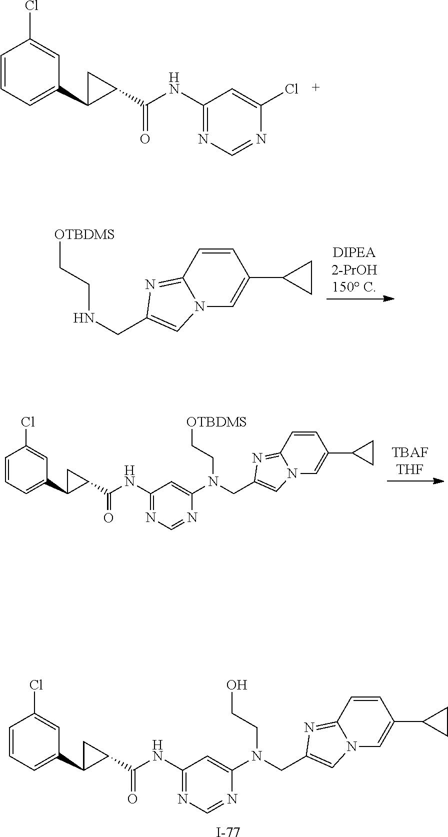

Synthesis of (1S,2S)—N-(6-((2-((tert-butyldimethylsilyl)oxy)ethyl)((6-cyclopropylimidazo[1,2-a]pyridin-2-yl)methyl)amino)pyrimidin-4-yl)-2-(3-chlorophenyl)cyclopropane-1-carboxamide. A mixture of (1S,2S)-2-(3-chlorophenyl)-N-(6-chloropyrimidin-4-yl)cyclopropane-1-carboxamide (0.040 g, 0.13 mmol), 2-((tert-butyldimethylsilyl)oxy)-N-((6-cyclopropylimidazo[1,2-a]pyridin-2-yl)methyl)ethan-1-amine (0.044 g, 0.13 mmol), and DIPEA (0.068 mL, 0.39 mmol) in iPrOH (1.5 mL) was stirred in a microwave at 150° C. for 1 hour. The reaction mixture was concentrated in vacuo, then the residue was purified by column chromatography on silica gel, eluting with a gradient of 0-20% 7N NH₃ in MeOH in DCM to give the title compound (0.070 g, 88%), which was used without further purification.

Synthesis of (1S,2S)-2-(3-chlorophenyl)-N-(6-(((6-cyclopropylimidazo[1,2-a]pyridin-2-yl)methyl)(2-hydroxyethyl)amino)pyrimidin-4-yl)cyclopropane-1-carboxamide (I-77). Using a similar procedure to that for (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide, the title compound was synthesized from (1S,2S)—N-(6-((2-((tert-butyldimethylsilyl)oxy)ethyl)((6-cyclopropylimidazo[1,2-a]pyridin-2-yl)methyl)amino)pyrimidin-4-yl)-2-(3-chlorophenyl)cyclopropane-1-carboxamide. ESI-MS (M+H): 503.4, ¹H NMR (400 MHz, DMSO) δ 10.72 (s, 1H), 8.36-8.30 (m, 2H), 7.68 (s, 1H), 7.49-7.30 (m, 5H), 7.22-7.18 (m, 1H), 7.04 (dd, J=1.5, 9.3 Hz, 1H), 5.25 (s, 1H), 4.84 (s, 2H), 3.68-3.67 (m, 4H), 2.49-2.38 (m, 2H), 2.01-1.93 (m, 1H), 1.56-1.42 (m, 2H), 0.99-0.93 (m, 2H), 0.74-0.69 (m, 2H).

### Example 78

### Synthesis of rac-(1S*,2S*)-2-(3-chlorophenyl)-N-(4-(((6-cyclopropylimidazo[1,2-a]pyridin-2-yl)methyl)amino)pyrimidin-5-yl)cyclopropane-1-carboxamide (I-78)

### Synthesis of rac-(1S*,2S*)-2-(3-chlorophenyl)-N-(4-chloropyrimidin-5-yl)cyclopropane-1-carboxamide

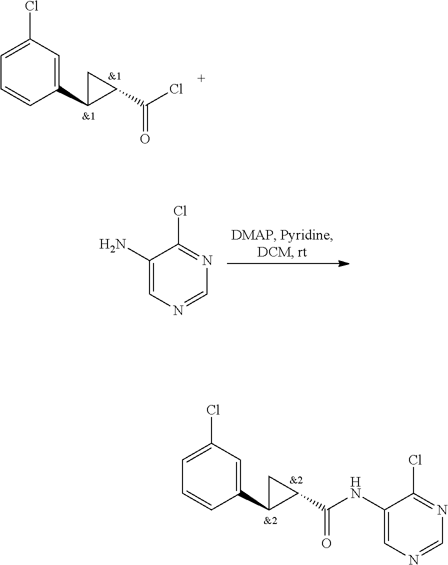 [SMILES: O=C(Cl)[C@H]1C[C@@H]1c1cccc(Cl)c1 ; Nc1cncnc1Cl ; O=C(Nc1cncnc1Cl)[C@H]1C[C@@H]1c1cccc(Cl)c1]

A mixture of 4-chloropyrimidin-5-amine (66 mg, 0.511 mmol), DMAP (6.2 mg, 0.0511 mmol), and pyridine (0.041 mL, 0.511 mmol) was added to a solution of rac-(1S*,2S*)-2-(3-chlorophenyl)cyclopropane-1-carbonyl chloride (110 mg, 0.511 mmol) in DCM (2 mL) at room temperature and stirred for 24 h. The mixture was diluted with DCM (25 mL) and washed with NH₄Cl (aq·sat·15 mL), water (10 mL), NaHCO₃ (aq·sat·15 mL), and water (10 mL), then dried (MgSO₄), filtered, and concentrated in vacuo to give the title compound (131 mg, 83%). ESI-MS (M+H)+: 310.0, ¹H NMR (400 MHz, DMSO) δ 10.26 (s, 1H), 9.22 (s, 1H), 8.82 (s, 1H), 7.38-7.33 (m, 1H), 7.31-7.28 (m, 2H), 7.23-7.19 (m, 1H), 2.49-2.46 (m, 1H), 1.59-1.47 (m, 2H). 1H missing, presumed under DMSO.

### Synthesis of rac-(1S*,2S*)-2-(3-chlorophenyl)-N-(4-(((6-cyclopropylimidazo[1,2-a]pyridin-2-yl)methyl)amino)pyrimidin-5-yl)cyclopropane-1-carboxamide (I-78)

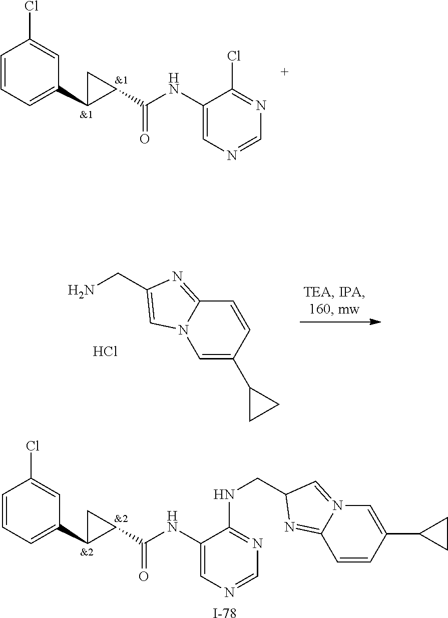

A mixture of rac-(1S*,2S*)-2-(3-chlorophenyl)-N-(4-chloropyrimidin-5-yl)cyclopropane-1-carboxamide (65 mg, 0.211 mmol), (6-cyclopropylimidazo[1,2-a]pyridin-2-yl)methanamine hydrochloride (71 mg, 0.316 mmol), TEA (0.15 mL, 1.05 mmol), and IPA (1 mL) was placed in a microwave vial, sealed, and heated to 160° C. under microwave conditions for 1 h. The mixture was diluted with water (25 mL) and extracted with EtOAc (3×25 mL). The organic phases were combined, dried (MgSO₄), filtered, and concentrated in vacuo. Purification by reverse phase preparative HPLC gave the title compound (7.5 mg, 7%) as an orange solid. ESI-MS (M+H)+: 459.3, ¹H NMR (400 MHz, DMSO) δ 9.59 (s, 1H), 8.33-8.28 (m, 3H), 7.65 (s, 1H), 7.40-7.24 (m, 6H), 7.18 (d, J=7.6 Hz, 1H), 6.96 (dd, J=1.0, 9.0 Hz, 1H), 4.66 (d, J=5.3 Hz, 2H), 2.46-2.39 (m, 1H), 2.17-2.12 (m, 1H), 1.94-1.86 (m, 1H), 1.54-1.39 (m, 2H), 0.94-0.87 (m, 2H), 0.68-0.63 (m, 2H).

### Synthesis of N⁴-((6-cyclopropylimidazo[1,2-a]pyridin-2-yl)methyl)-5-fluoropyridine-2,4-diamine

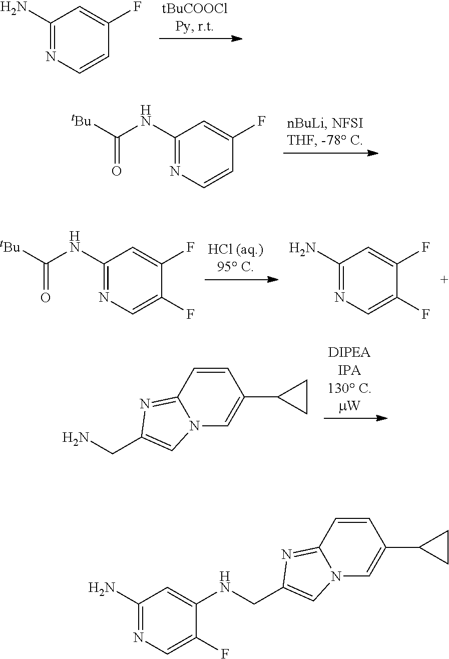 [SMILES: Nc1cc(F)ccn1 ; CC(C)(C)C(=O)Nc1cc(F)ccn1 ; CC(C)(C)C(=O)Nc1cc(F)c(F)cn1 ; Nc1cc(F)c(F)cn1 ; NCc1cn2cc(C3CC3)ccc2n1 ; Nc1cc(NCc2cn3cc(C4CC4)ccc3n2)c(F)cn1]

Synthesis of N-(4-fluoropyridin-2-yl)pivalamide. Pivaloyl chloride (1.6 mL, 13 mmol) was added dropwise to a stirred solution of 4-fluoropyridin-2-amine (1.0 g, 8.9 mmol) in pyridine (4.0 mL). The reaction was then stirred at room temperature for 18 h. The reaction was partitioned between EtOAc (20 mL) and water (30 mL), the layers were separated, and the aqueous layer was further extracted with EtOAc (2×20 mL). The organic layers were combined, dried (MgSO₄), and concentrated in vacuo. The residue was purified by column chromatography on silica gel, eluting with a gradient of 0-5% MeOH in DCM to give the title compound (1.3 g, 75%). ESI-MS (M+H)+: 197.1, ¹H NMR (400 MHz, CDCl₃) δ 8.38-8.28 (br s, 1H), 8.20 (dd, J=5.6, 8.6 Hz, 1H), 8.07 (dd, J=2.5, 11.4 Hz, 1H), 6.80-6.76 (m, 1H), 1.32 (s, 9H).

Synthesis of N-(4,5-difluoropyridin-2-yl)pivalamide. nBuLi (2.2 M in hexanes, 2.8 mL, 6.1 mmol) was added dropwise to a stirred solution of N-(4-fluoropyridin-2-yl)pivalamide (500 mg, 2.6 mmol) in THF (15 mL) at −78° C. and stirred for 30 min. NFSI (1.6 g, 5.1 mmol) in THF (5.0 mL) was then added dropwise, and the reaction was stirred at −78° C. for a further 20 min and then allowed to warm to room temperature. The reaction was quenched with NH₄Cl (sat·aq·, 15 mL) and extracted with EtOAc (3×30 mL). The organic layers were combined, dried (MgSO₄), and concentrated in vacuo. The residue was purified by column chromatography on silica gel, eluting with a gradient of 0-5% MeOH in DCM to give the title compound (400 mg, 73%). ESI-MS (M+H)+: 215.1, ¹H NMR (400 MHz, CDCl₃) δ 8.14 (m, 1H), 7.68 (br s, 1H), 7.05-6.99 (m, 1H), 1.35 (s, 9H).

Synthesis of 4,5-difluoropyridin-2-amine. N-(4,5-difluoropyridin-2-yl)pivalamide (400 mg, 1.9 mmol) was suspended in concentrated HCl (33% aq·, 5.0 mL) and heated at 95° C. for 2 h. The reaction was cooled to room temperature and then poured over ice (25 g). The reaction was then taken to basic pH using NaOH (2.0 M aq·) and extracted with EtOAc (3×30 mL). The organic layers were combined, dried (MgSO₄), and concentrated in vacuo to give the title compound (150 mg, 62%). ESI-MS (M+H)+: 131.1, ¹H NMR (400 MHz, CDCl₃) δ 7.78 (m, 1H), 6.54-6.49 (m, 1H), 4.77-4.68 (br s, 2H).

Synthesis of N⁴-((6-cyclopropylimidazo[1,2-a]pyridin-2-yl)methyl)-5-fluoropyridine-2,4-diamine. A solution of 4,5-difluoropyridin-2-amine (0.120 g, 0.92 mmol), (6-cyclopropylimidazo[1,2-a]pyridin-2-yl)methanamine (0.31 g, 1.4 mmol) and DIPEA (0.56 mL, 3.2 mmol) in iPrOH (5.0 mL) was heated to 130° C. in a microwave for 2.5 h. The mixture was cooled to room temperature and concentrated in vacuo. The residue was purified by column chromatography on silica gel, eluting with a gradient of 0-5% (7 N NH₃ in MeOH) in DCM to give the title compound (0.170 g, 62%). ESI-MS (M+H)+: 298.2, ¹H NMR (400 MHz, DMSO) δ 8.34 (s, 1H), 7.66 (s, 1H), 7.44-7.38 (m, 2H), 7.01 (dd, J=1.8, 9.3 Hz, 1H), 6.61 (dd, J=5.2, 5.2 Hz, 1H), 6.11 (dd, J=5.9, 5.9 Hz, 1H), 5.61 (s, 2H), 4.45 (d, J=6.1 Hz, 2H), 1.99-1.91 (m, 1H), 0.98-0.92 (m, 2H), 0.73-0.68 (m, 2H).

### Synthesis of 1-(2-(((2-aminopyridin-4-yl)amino)methyl)-6-cyclopropylimidazo[1,2-a]pyridin-8-yl)pyrrolidin-2-one

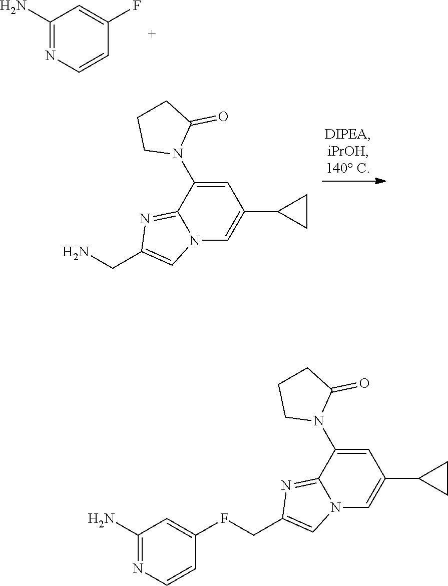

Synthesis of 1-(2-(((2-aminopyridin-4-yl)amino)methyl)-6-cyclopropylimidazo[1,2-a]pyridin-8-yl)pyrrolidin-2-one. A solution of 4-fluoropyridin-2-amine (0.058 g, 0.52 mmol), 1-(2-(aminomethyl)-6-cyclopropylimidazo[1,2-a]pyridin-8-yl)pyrrolidin-2-one (0.12 g, 0.55 mmol), and DIPEA (0.33 mL, 1.9 mmol) in iPrOH (5.0 mL) was heated to 140° C. in a microwave for 14 h. The mixture was cooled to room temperature and concentrated in vacuo. The residue was purified by column chromatography on silica gel, eluting with a gradient of 0-20% (7 N NH₃ in MeOH) in DCM to give the title compound (0.070 g, 34%). ESI-MS (M+H)+: 377, ¹H NMR (400 MHz, DMSO) δ 8.29 (d, J=1.3 Hz, 1H), 7.65 (s, 1H), 7.46 (d, J=5.8 Hz, 1H), 6.98 (d, J=1.6 Hz, 1H), 6.62 (m, 1H), 5.93 (dd, J=2.1, 5.8 Hz, 1H), 5.77 (s, 1H), 5.62 (d, J=2.0 Hz, 1H), 5.33 (s, 2H), 4.89-4.83 (m, 1H), 4.31 (dd, J=1.6, 5.6 Hz, 2H), 2.50-2.47 (m, 3H), 1.97-1.89 (m, 1H), 1.76-1.66 (m, 1H), 0.96-0.92 (m, 2H), 0.69-0.64 (m, 2H).

The compounds in Table 10 were synthesized using a similar procedure to that used for 1-(2-(((2-aminopyridin-4-yl)amino)methyl)-6-cyclopropylimidazo[1,2-a]pyridin-8-yl)pyrrolidin-2-one from 4-fluropyridin-2-amine or 4-fluoropyrimidin-2-amine and an appropriate coupling partner.

TABLE 10

| | Coupling Partner/ |
|---|---|
| Compound | Analytical Data |
| 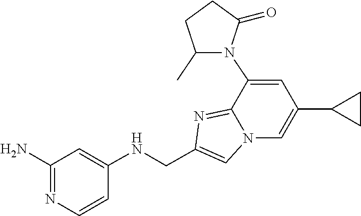 [SMILES: CC1CCC(=O)N1c1cc(C2CC2)cn2cc(CNc3ccnc(N)c3)nc12] | 1-(2-(aminomethyl)-6-cyclopropylimidazo[1,2- a]pyridin-8-yl)-5-methylpyrrolidin-2-one ESI-MS (M + H)+: 377.4, ¹H NMR (400 MHz, DMSO) δ 8.29 (d, J = 1.3 Hz, 1H), 7.65 (s, 1H), 7.46 (d, J = 5.8 Hz, 1H), 6.98 (d, J = 1.6 Hz, 1H), 6.62 (dd, J = 5.7, 5.7 Hz, 1H), 5.93 (dd, J = 2.1, 5.8 Hz, 1H), 5.77 (s, 1H), 5.62 (d, J = 2.0 Hz, 1H), 5.33 (s, 2H), 4.89-4.83 (m, 1H), 4.31 (dd, J = 1.6, 5.6 Hz, 2H), 2.50-2.47 (m, 3H), 1.97-1.89 (m, 1H), 1.76-1.66 (m, 1H), 1.01 (d, 3H), 0.96-0.92 (m, 2H), 0.69-0.64 (m, 2H). |
| 1-(2-(((2-aminopyridin-4- | |
| yl)amino)methyl)-6- | |
| cyclopropylimidazo[1,2-a]pyridin-8-yl)- | |
| 5-methylpyrrolidin-2-one | |
| 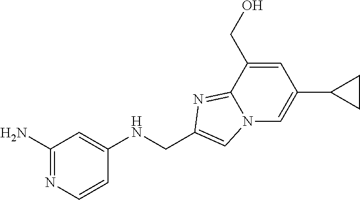 [SMILES: Nc1cc(NCc2cn3cc(C4CC4)cc(CO)c3n2)ccn1] | (2-(aminomethyl)-6-cyclopropylimidazo[1,2- a]pyridin-8-yl)methanol ¹H NMR (400 MHz, DMSO) δ 8.25 (s, 1H), 7.65 (s, 1H), 7.50 (d, J = 5.8 Hz, 1H), 7.03 (d, J = 1.5 Hz, 1H), 6.61 (m, 1H), 5.96 (dd, J = 2.1, 5.9 Hz, 1H), 5.65 (d, J = 2.0 Hz, 1H), 5.38-5.32 (m, 3H), 4.83 (d, J = 5.3 Hz, 2H), 4.34 (d, J = 5.8 Hz, 2H), 2.02-1.93 (m, 1H), 0.99- 0.93 (m, 2H), 0.72-0.67 (m, 2H). |
| (2-(((2-aminopyridin-4- | |
| yl)amino)methyl)-6- | |
| cyclopropylimidazo[1,2-a]pyridin-8- | |
| yl)methanol | |
| 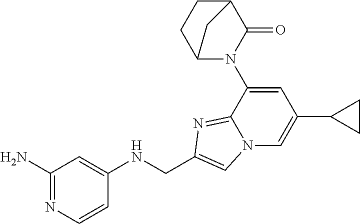 [SMILES: Nc1cc(NCc2cn3cc(C4CC4)cc(N4C(=O)C5CCC4C5)c3n2)ccn1] | 2-(2-(aminomethyl)-6-cyclopropylimidazo[1,2- a]pyridin-8-yl)-2-azabicyclo[2.2.1]heptan-3-one ESI-MS (M + H)+: 297.3, ¹H NMR (400 MHz, DMSO) δ 8.21 (d, J = 1.0 Hz, 1H), 7.68 (s, 1H), 7.51 (d, J = 5.8 Hz, 1H), 7.36 (d, J = 1.5 Hz, 1H), 6.62 (m, 1H), 5.98 (dd, J = 2.0, 5.8 Hz, 1H), 5.66 (d, J = 2.0 Hz, 1H), 5.51 (s, 1H), 5.33 (s, 2H), 4.36 (d, J = 5.8 Hz, 2H), 3.22 (d, J = 5.1 Hz, 1H), 2.89 (d, J = 2.5 Hz, 1H), 2.08 (d, J = 9.6 Hz, 1H), 2.03-1.79 (m, 2H), 1.71-1.59 (m, 1H), 0.98- 0.92 (m, 2H), 0.70-0.64 (m, 2H). |
| (2-(((2-aminopyridin-4- | |
| yl)amino)methyl)-6- | |
| cyclopropylimidazo[1,2-a]pyridin-8- | |
| yl)methanol | |
| 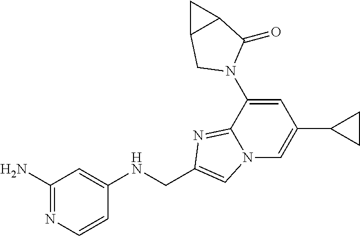 [SMILES: Nc1cc(NCc2cn3cc(C4CC4)cc(N4CC5CC5C4=O)c3n2)ccn1] | 3-(2-(aminomethyl)-6-cyclopropylimidazo[1,2- a]pyridin-8-yl)-3-azabicyclo[3.1.0]hexan-2-one ¹H NMR (400 MHz, CDCl₃): δ ppm 7.72 (dd, J = 0.7, 1.5 Hz, 1H), 7.69 (d, J = 6.0 Hz, 1H), 7.37 (s, 1H), 7.11 (d, J = 1.5 Hz, 1H), 6.04 (dd, J = 2.1, 6.0 Hz, 1H), 5.73 (d, J = 2.1 Hz, 1H), 4.82 (t, J = 5.5 Hz, 1H), 4.49 (dd, J = 5.8, 10.2 Hz, 1H), 4.44 (d, J = 5.5 Hz, 2H), 4.11 (dd, J = 1.5, 10.4 Hz, 1H), 2.15-2.03 (m, 2H), 1.91-1.83 (m, 1H), 1.29-1.23 (m, 1H), 1.00-0.92 (m, 3H), 0.69- 0.65 (m, 2H) |
| (3-(((2-aminopyridin-4- | |
| yl)amino)methyl)-6- | |
| cyclopropylimidazo[1,2-a]pyridin-8-yl)- | |
| 3-azabicyclo[3.1.0]hexan-2-one | |
| 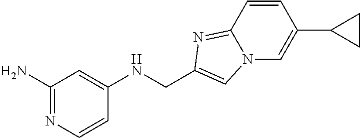 [SMILES: Nc1cc(NCc2cn3cc(C4CC4)ccc3n2)ccn1] | (6-cyclopropylimidazo[1,2-a]pyridin-2- yl)methanamine ESI-MS (M + H)+: 280.2, ¹H NMR (400 MHz, CDCl₃) δ 7.85 (d, J = 7.3 Hz, 1H), 7.43 (dd, J = 6.3, 9.3 Hz, 1H), 7.73 (d, 1H), 7.39 (s, 1H), 6.93 (m, 1H), 6.04 (d, 1H), 5.73 (s, 1H), 4.47 (d, J = 5.3 Hz, 2H), 1.93-1.84 (m, 1H), 0.99-0.95 (m, 2H), 0.69-0.64 (m, 2H). |
| N⁴-((6-cyclopropylimidazo[1,2-a]pyridin- | |
| 2-yl)methyl)pyridine-2,4-diamine | |
| 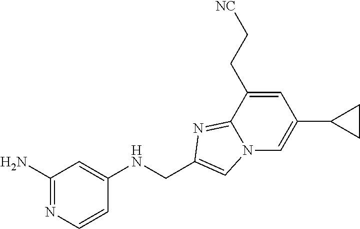 [SMILES: N#CCCc1cc(C2CC2)cn2cc(CNc3ccnc(N)c3)nc12] | 3-(2-(aminomethyl)-6-cyclopropylimidazo[1,2- a]pyridin-8-yl)propanenitrile ESI-MS [M + H ]+: 333.1 |
| 3-(2-(((2-aminopyridin-4- | |
| yl)amino)methyl)-6- | |
| cyclopropylimidazo[1,2-a]pyridin-8- | |
| yl)propanenitrile | |
| 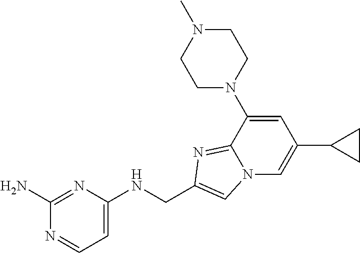 [SMILES: CN1CCN(c2cc(C3CC3)cn3cc(CNc4ccnc(N)n4)nc23)CC1] | (6-cyclopropyl-8-(4-methylpiperazin-1- yl)imidazo[1,2-a]pyridin-2-yl)methanamine ESI-MS (M + H)+: 379.2 |
| N4-((6-cyclopropyl-8-(4- | |
| methylpiperazin-1-yl)imidazo[1,2- | |
| a]pyridin-2-yl)methyl)pyrimidine-2,4- | |
| diamine | |

### Examples 79-81

### Synthesis of rac-(1S*2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide (I-79)

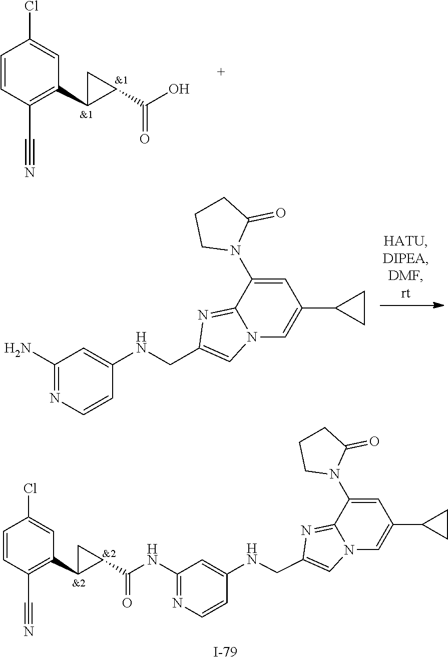

DIPEA (0.32 mL, 1.8 mmol) was added to a stirred mixture of 1-(2-(((2-aminopyridin-4-yl)amino)methyl)-6-cyclopropylimidazo[1,2-a]pyridin-8-yl)pyrrolidin-2-one (140 mg, 0.39 mmol), rac-(1S*,2S*)-2-(5-chloro-2-cyanophenyl)cyclopropane-1-carboxylic acid (82 mg, 0.39 mmol), and HATU (180 mg, 0.48 mmol) in DMF (4.0 mL). The mixture was then stirred at room temperature for 18 h. The mixture was diluted with EtOAc (50 mL) and washed with water (2×30 mL) and brine (sat·aq·, 30 mL). The organic layers were dried (MgSO₄) and concentrated in vacuo. The residue was purified by reverse phase preparative HPLC to give the title compound (19 mg, 9%) as a formic acid salt (mixture of enantiomers).

ESI-MS (M+H)+: 566.4, ¹H NMR (400 MHz, CDCl₃) δ 8.50 (s, 1H), 7.81 (s, 2H), 7.61 (d, J=6.8 Hz, 1H), 7.57-7.52 (m, 2H), 7.30 (dd, J=1.9, 8.3 Hz, 1H), 7.19 (dd, J=1.8, 4.9 Hz, 2H), 6.38 (dd, J=2.4, 6.8 Hz, 1H), 4.55 (d, J=5.0 Hz, 2H), 4.21-4.14 (m, 2H), 2.95-2.89 (m, 1H), 2.66-2.61 (m, 2H), 2.42-2.36 (m, 1H), 2.30-2.21 (m, 2H), 1.94-1.78 (m, 2H), 1.45 (ddd, J=4.8, 6.5, 8.4 Hz, 1H), 1.00-0.95 (m, 2H), 0.73-0.68 (m, 2H).

The mixture was separated using SFC (YMC Amylose-C 10×250 mm, 5 μm 55/45 MeOH (0.1% DEA)/CO₂, 15 mL/min, 120 bar, 40° C.) to give two enantiomers: (1S,2S)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide and (1R,2R)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide.

First eluting isomer (I-80): ESI-MS (M+H)+: 566.4, ¹H NMR (400 MHz, CDCl₃) δ 8.50 (s, 1H), 7.81 (s, 2H), 7.61 (d, J=6.8 Hz, 1H), 7.57-7.52 (m, 2H), 7.30 (dd, J=1.9, 8.3 Hz, 1H), 7.19 (dd, J=1.8, 4.9 Hz, 2H), 6.38 (dd, J=2.4, 6.8 Hz, 1H), 4.55 (d, J=5.0 Hz, 2H), 4.21-4.14 (m, 2H), 2.95-2.89 (m, 1H), 2.66-2.61 (m, 2H), 2.42-2.36 (m, 1H), 2.30-2.21 (m, 2H), 1.94-1.78 (m, 2H), 1.45 (ddd, J=4.8, 6.5, 8.4 Hz, 1H), 1.00-0.95 (m, 2H), 0.73-0.68 (m, 2H). RT=6.43 min, 99.9% e.e. Formic acid salt.

Second eluting isomer (I-81): ESI-MS (M+H)+: 566.4, ¹H NMR (400 MHz, CDCl₃) δ 8.50 (s, 1H), 7.81 (s, 2H), 7.61 (d, J=6.8 Hz, 1H), 7.57-7.52 (m, 2H), 7.30 (dd, J=1.9, 8.3 Hz, 1H), 7.19 (dd, J=1.8, 4.9 Hz, 2H), 6.38 (dd, J=2.4, 6.8 Hz, 1H), 4.55 (d, J=5.0 Hz, 2H), 4.21-4.14 (m, 2H), 2.95-2.89 (m, 1H), 2.66-2.61 (m, 2H), 2.42-2.36 (m, 1H), 2.30-2.21 (m, 2H), 1.94-1.78 (m, 2H), 1.45 (ddd, J=4.8, 6.5, 8.4 Hz, 1H), 1.00-0.95 (m, 2H), 0.73-0.68 (m, 2H). RT=8.17 min, 98.5% e.e.

### Examples 82-84

### Synthesis of rac-(1S*,2S*)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-82)

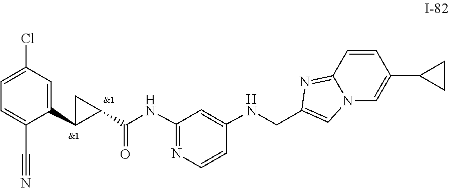 [SMILES: N#Cc1ccc(Cl)cc1[C@H]1C[C@@H]1C(=O)Nc1cc(NCc2cn3cc(C4CC4)ccc3n2)ccn1]

Using a similar procedure to that used for rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the title compound was prepared using rac-(1S*,2S*)-2-(5-chloro-2-cyanophenyl)cyclopropane-1-carboxylic acid (57 mg, 0.26 mmol) and N⁴-((6-cyclopropylimidazo[1,2-a]pyridin-2-yl)methyl)pyridine-2,4-diamine (100 mg, 0.23 mmol), then purified by preparative LCMS to give the title compound (19 mg, 17%) as a mixture of enantiomers. ESI-MS (M+H): +483.3, ¹H NMR (400 MHz, DMSO) δ 10.35 (s, 1H), 8.30 (s, 1H), 7.85 (d, J=8.3 Hz, 1H), 7.76 (d, J=5.6 Hz, 1H), 7.63 (s, 1H), 7.51 (dd, J=2.3, 8.3 Hz, 1H), 7.45 (s, 1H), 7.39 (s, 1H), 7.37 (d, J=5.7 Hz, 1H), 7.11 (t, J=5.8 Hz, 1H), 6.97 (dd, J=1.8, 9.3 Hz, 1H), 6.33 (dd, J=2.3, 5.8 Hz, 1H), 4.35 (d, J=5.8 Hz, 2H), 2.59-2.52 (m, 1H), 1.90 (tt, J=3.9, 9.3 Hz, 1H), 1.56-1.51 (m, 2H), 0.90 (ddd, J=4.3, 6.3, 8.4 Hz, 2H), 0.66 (td, J=4.7, 6.3 Hz, 2H). One CH of cyclopropyl group obscured by DMSO peak.

The mixture was separated using SFC (YMC Amylose-C 10×250 mm, 5 μm 55/45 MeOH (0.1% DEA)/CO₂, 15 mL/min, 120 bar, 40° C.) to give two enantiomers: (1R,2R)-2-(5-chloro-2-cyanophenyl)-N-(4-(((6-cyclopropylimidazo[1,2-a]pyridin-2-yl)methyl)amino)pyridin-2-yl)cyclopropane-1-carboxamide and (1S,2S)-2-(5-chloro-2-cyanophenyl)-N-(4-(((6-cyclopropylimidazo[1,2-a]pyridin-2-yl)methyl)amino)pyridin-2-yl)cyclopropane-1-carboxamide.

First eluting isomer, (1R,2R)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-83): ESI-MS (M+H)+: 483.2, ¹H NMR (400 MHz, DMSO) δ 10.36 (s, 1H), 8.30 (s, 1H), 7.85 (d, J=8.3 Hz, 1H), 7.76 (d, J=5.8 Hz, 1H), 7.63 (s, 1H), 7.51 (dd, J=2.0, 8.3 Hz, 1H), 7.45 (s, 1H), 7.39-7.36 (m, 2H), 7.11 (dd, J=5.9, 5.9 Hz, 1H), 6.97 (dd, J=1.9, 9.2 Hz, 1H), 6.33 (dd, J=2.0, 5.8 Hz, 1H), 4.35 (d, J=5.8 Hz, 2H), 2.59-2.54 (m, 1H), 1.94-1.86 (m, 1H), 1.56-1.51 (m, 2H), 0.90 (ddd, J=4.3, 6.3, 8.3 Hz, 2H), 0.68-0.63 (m, 2H). One CH of cyclopropyl group obscured by DMSO peak. RT=5.52 min, 99% e.e.

Second eluting isomer, (1S,2S)-2-(5-chloro-2-cyanophenyl)-N-(4-(((6-cyclopropylimidazo[1,2-a]pyridin-2-yl)methyl)amino)pyridin-2-yl)cyclopropane-1-carboxamide (I-84): ESI-MS (M+H)+: 483.2, ¹H NMR (400 MHz, DMSO) δ 10.36 (s, 1H), 8.30 (s, 1H), 7.85 (d, J=8.3 Hz, 1H), 7.76 (d, J=5.8 Hz, 1H), 7.63 (s, 1H), 7.51 (dd, J=2.0, 8.3 Hz, 1H), 7.45 (s, 1H), 7.39-7.36 (m, 2H), 7.11 (dd, J=5.8, 5.8 Hz, 1H), 6.97 (dd, J=1.8, 9.3 Hz, 1H), 6.33 (dd, J=2.0, 5.8 Hz, 1H), 4.36 (d, J=5.8 Hz, 2H), 2.60-2.52 (m, 1H), 1.94-1.86 (m, 1H), 1.54 (dd, J=7.5, 7.5 Hz, 2H), 0.90 (ddd, J=4.4, 6.3, 8.4 Hz, 2H), 0.68-0.63 (m, 2H). RT=12.87 min, 98.5% e.e.

### Example 85

### Synthesis of (1S,2S)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide (I-85)

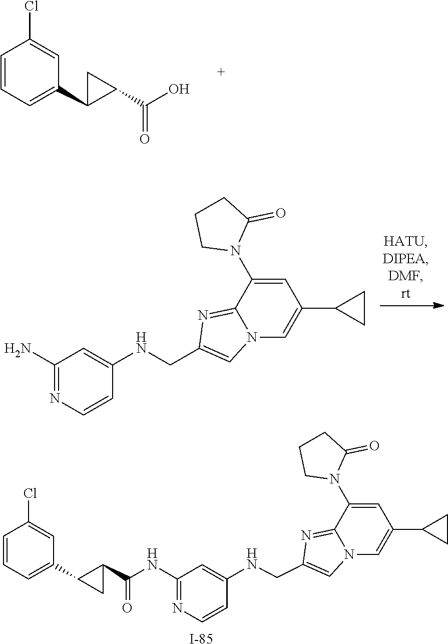

A mixture of 1-(2-(((2-aminopyridin-4-yl)amino)methyl)-6-cyclopropylimidazo[1,2-a]pyridin-8-yl)pyrrolidin-2-one (183 mg, 0.51 mmol), (1S,2S)-2-(3-chlorophenyl)cyclopropane-1-carboxylic acid (95 mg, 0.48 mmol), HATU (238 mg, 0.63 mmol), and DIPEA (0.42 mL, 2.40 mmol) in DMF (4.0 mL) was stirred at room temperature for 18 h. Water (50 mL) and NaHCO₃ (sat·aq·, 50 mL) were added, and the mixture was extracted with DCM (3×50 mL). The combined organics were dried over MgSO₄, filtered, and concentrated in vacuo. The residue was purified by column chromatography on silica gel, eluting with 0-20% (7N NH₃ in MeOH) in DCM to give the title compound (120 mg, 46%). ESI-MS (M+H)+: 541, ¹H NMR (400 MHz, DMSO) δ 10.36 (s, 1H), 8.30 (d, J=1.3 Hz, 1H), 7.81 (d, J=5.8 Hz, 1H), 7.72 (s, 1H), 7.52-7.49 (m, 1H), 7.36 (dd, J=8.0, 8.0 Hz, 1H), 7.31-7.28 (m, 2H), 7.21-7.17 (m, 3H), 6.37 (dd, J=2.0, 5.8 Hz, 1H), 4.41 (d, J=5.8 Hz, 2H), 4.21 (dd, J=7.1, 7.1 Hz, 2H), 2.54-2.50 (m, 2H), 2.46-2.37 (m, 2H), 2.23-2.14 (m, 2H), 2.01-1.93 (m, 1H), 1.53-1.46 (m, 1H), 1.43-1.37 (m, 1H), 0.97 (ddd, J=4.3, 6.3, 8.3 Hz, 2H), 0.72-0.67 (m, 2H).

Using a similar procedure to that used for (1S,2S)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the compounds in Table 11 were prepared using rac-(1S*,2S*)-2-(3-chlorophenyl)cyclopropane-1-carboxylic acid or (1S,2S)-2-(3-chlorophenyl)cyclopropane-1-carboxylic acid and an appropriate coupling partner.

TABLE 11

| | | Coupling Partner/ |
|---|---|---|
| Example | Compound | Analytical Data |
| Example 86 | 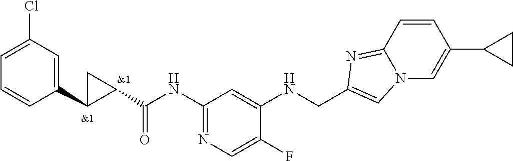 [SMILES: O=C(Nc1cc(NCc2cn3cc(C4CC4)ccc3n2)c(F)cn1)[C@H]1C[C@@H]1c1cccc(Cl)c1] rac-(1S*,2S*)-2-(3-chlorophenyl)-N-(4- (((6-cyclopropylimidazo[1,2-a]pyridin-2- yl)methyl)amino)-5-fluoropyridin-2- yl)cyclopropane-1-carboxamide; formic acid salt (I-86) | N⁴-((6-Cyclopropylimidazo[1,2- a]pyridin-2-yl)methyl)-5- fluoropyridine-2,4-diamine ESI-MS (M + H)+: 476.3, ¹H NMR (400 MHz, DMSO) δ 10.17 (s, 1H), 8.33 (s, 1H), 7.75 (d, J = 5.5 Hz, 1H), 7.68 (s, 1H), 7.42 (d, J = 9.3 Hz, 1H), 7.39-7.34 (m, 1H), 7.31 (d, J = 1.3 Hz, 2H), 7.29-7.29 (m, 2H), 7.20 (d, J = 7.7 Hz, 1H), 7.15 (dd, J = 6.1, 6.1 Hz, 1H), 7.01 (dd, J = 1.8, 9.3 Hz, 1H), 6.65 (dd, J = 6.0, 6.0 Hz, 1H), 4.50 (d, J = 5.9 Hz, 2H), 2.58 (s, 1H), 2.44- 2.38 (m, 1H), 2.27-2.22 (m, 1H), 1.98-1.90 (m, 1H), 1.52-1.41 (m, 2H), 0.97-0.91 (m, 2H), 0.72-0.67 |
| | | (m, 2H). |
| Example 87 | 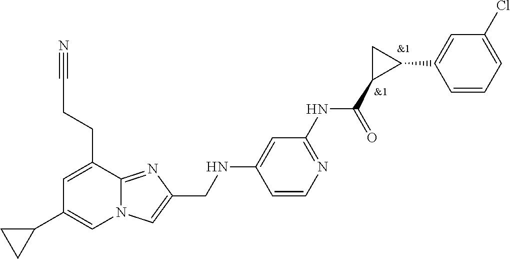 [SMILES: N#CCCc1cc(C2CC2)cn2cc(CNc3ccnc(NC(=O)[C@H]4C[C@@H]4c4cccc(Cl)c4)c3)nc12] | 3-(2-(((2-aminopyridin-4- yl)amino)methyl)-6- cyclopropylimidazo[1,2-a]pyridin-8- yl)propanenitrile ESI-MS [M + H ]+: 511.2. ¹H NMR (400 MHz, DMSO) δ 10.31 (s, 1H), 8.24 (s, 1H), 7.75 (d, J = 5.7 Hz, 1H), 7.64 (s, 1H), 7.44 (s, 1H), 7.36-7.26 (m, 1H), 7.27-7.17 (m, 2H), 7.16- 7.04 (m, 3H), 6.92 (s, 1H), 6.32 (d, J = 5.7 Hz, 1H), 4.37 (d, J = 5.6 Hz, 2H), 3.17-3.11 (m, 2H), 3.10-3.01 (m, 2H), 2.45-2.28 (m, 2H), 1.94- 1.84 (m, 1H), 1.49-1.39 (m, 1H), 1.39-1.29 (m, 1H), 0.91 (d, J = 8.3 Hz, 2H), 0.67 (d, J = 5.1 Hz, 2H). |
| | rac-(1S*,2S*)-2-(3-chlorophenyl)-N-(4- | |
| | (((8-(2-cyanoethyl)-6- | |
| | cyclopropylimidazo[1,2-a]pyridin-2- | |
| | yl)methyl)amino)pyridin-2- | |
| | yl)cyclopropane-1-carboxamide (I-87) | |
| Example 88 | 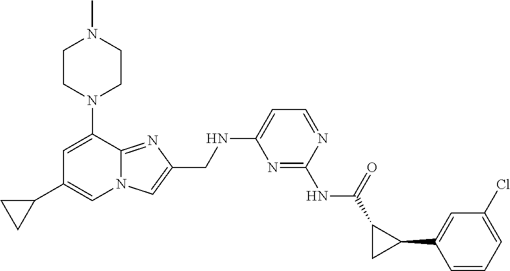 [SMILES: CN1CCN(c2cc(C3CC3)cn3cc(CNc4ccnc(NC(=O)[C@H]5C[C@@H]5c5cccc(Cl)c5)n4)nc23)CC1] | N4-((6-cyclopropyl-8-(4- methylpiperazin-1-yl)imidazo[1,2- a]pyridin-2-yl)methyl)pyrimidine- 2,4-diamine ESI-MS [M + H]+: 557.2. ¹H NMR (400 MHz, DMSO) δ 10.23 (s, 1H), 7.88 (s, 2H), 7.61 (s, 1H), 7.39-7.16 (m, 3H), 7.12 (d, J = 7.7 Hz, 1H), 6.24 (d, J = 5.9 Hz, 1H), 6.18 (s, 1H), 4.50 (s, 2H), 3.50-3.40 (m, 4H), 2.77- 2.67 (m, 4H), 2.53 (s, 3H), 2.43 (s, 2H), 1.99-1.78 (m, 1H), 1.56-1.43 (m, 1H), 1.42-1.31 (m, 1H), 0.99- 0.76 (m, 2H), 0.69-0.65 (m, 2H). |
| | (1S,2S)-2-(3-chlorophenyl)-N-(4-(((6- | |
| | cyclopropyl-8-(4-methylpiperazin-1- | |
| | yl)imidazo[1,2-a]pyridin-2- | |
| | yl)methyl)amino)pyrimidin-2- | |
| | yl)cyclopropane-1-carboxamide (I-88) | |

### Examples 89-91

### Synthesis of (1S,2S)-2-(3-chlorophenyl)-N-(4-(((6-cyclopropyl-8-(2-methyl-5-oxopyrrolidin-1-yl)imidazo[1,2-a]pyridin-2-yl)methyl)amino)pyridin-2-yl)cyclopropane-1-carboxamide (I-89)

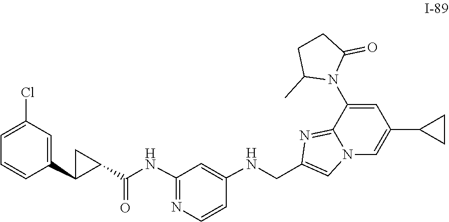 [SMILES: CC1CCC(=O)N1c1cc(C2CC2)cn2cc(CNc3ccnc(NC(=O)[C@H]4C[C@@H]4c4cccc(Cl)c4)c3)nc12]

Using a similar procedure to that used for rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the title compound was prepared using (S,S)-2-(3-chlorophenyl)cyclopropane-1-carboxylic acid (35 mg, 0.18 mmol) and N⁴-((6-cyclopropylimidazo[1,2-a]pyridin-2-yl)methyl)pyridine-2,4-diamine (70 mg, 0.19 mmol), then purified by preparative LCMS to give the title compound (20 mg, 20%) as a mixture of diastereomers. ESI-MS (M+H)+: 555.4, ¹H NMR (400 MHz, DMSO) δ 10.33 (s, 1H), 8.29 (d, J=1.4 Hz, 1H), 7.77 (d, J=5.8 Hz, 1H), 7.68 (s, 1H), 7.46 (s, 1H), 7.33 (dd, J=7.6, 7.6 Hz, 1H), 7.26 (d, J=7.3 Hz, 2H), 7.15 (d, J=7.7 Hz, 2H), 6.99 (d, J=1.6 Hz, 1H), 6.33 (dd, J=2.0, 5.8 Hz, 1H), 4.89 (dd, J=6.1, 12.3 Hz, 1H), 4.37 (d, J=5.8 Hz, 2H), 2.49-2.33 (m, 4H), 1.98-1.90 (m, 1H), 1.76-1.66 (m, 1H), 1.49-1.43 (m, 1H), 1.39-1.32 (m, 1H), 1.01 (d, J=6.4 Hz, 3H), 0.97-0.92 (m, 2H), 0.69-0.64 (m, 2H). 1 proton hidden under DMSO signal.

The mixture was separated using SFC (LUX Cellulose-4 10×250 mm, 5 μm 55/45 MeOH (0.1% DEA)/CO₂, 15 mL/min, 120 bar, 40° C.) to give two diastereomers: (1S,2S)-2-(3-chlorophenyl)-N-(4-(((6-cyclopropyl-8-((S)-2-methyl-5-oxopyrrolidin-1-yl)imidazo[1,2-a]pyridin-2-yl)methyl)amino)pyridin-2-yl)cyclopropane-1-carboxamide and (1S,2S)-2-(3-chlorophenyl)-N-(4-(((6-cyclopropyl-8-((R)-2-methyl-5-oxopyrrolidin-1-yl)imidazo[1,2-a]pyridin-2-yl)methyl)amino)pyridin-2-yl)cyclopropane-1-carboxamide.

First eluting isomer (I-90): ESI-MS (M+H)+: 555.4, ¹H NMR (400 MHz, DMSO) δ 10.33 (s, 1H), 8.29 (d, J=1.4 Hz, 1H), 7.77 (d, J=5.8 Hz, 1H), 7.68 (s, 1H), 7.46 (s, 1H), 7.33 (dd, J=7.6, 7.6 Hz, 1H), 7.26 (d, J=7.3 Hz, 2H), 7.15 (d, J=7.7 Hz, 2H), 6.99 (d, J=1.6 Hz, 1H), 6.33 (dd, J=2.0, 5.8 Hz, 1H), 4.89 (dd, J=6.1, 12.3 Hz, 1H), 4.37 (d, J=5.8 Hz, 2H), 2.49-2.33 (m, 4H), 1.98-1.90 (m, 1H), 1.76-1.66 (m, 1H), 1.49-1.43 (m, 1H), 1.39-1.32 (m, 1H), 1.01 (d, J=6.4 Hz, 3H), 0.97-0.92 (m, 2H), 0.69-0.64 (m, 2H). 1 proton hidden under DMSO. RT=14.42 min, 97.71% e.e.

Second eluting isomer (I-91): ESI-MS (M+H)+: 555.4, ¹H NMR (400 MHz, DMSO) δ 10.33 (s, 1H), 8.29 (d, J=1.4 Hz, 1H), 7.77 (d, J=5.8 Hz, 1H), 7.68 (s, 1H), 7.46 (s, 1H), 7.33 (dd, J=7.6, 7.6 Hz, 1H), 7.26 (d, J=7.3 Hz, 2H), 7.15 (d, J=7.7 Hz, 2H), 6.99 (d, J=1.6 Hz, 1H), 6.33 (dd, J=2.0, 5.8 Hz, 1H), 4.89 (dd, J=6.1, 12.3 Hz, 1H), 4.37 (d, J=5.8 Hz, 2H), 2.49-2.33 (m, 4H), 1.98-1.90 (m, 1H), 1.76-1.66 (m, 1H), 1.49-1.43 (m, 1H), 1.39-1.32 (m, 1H), 1.01 (d, J=6.4 Hz, 3H), 0.97-0.92 (m, 2H), 0.69-0.64 (m, 2H). 1 proton hidden under DMSO. RT=18.25 min, 96.6% e.e.

### Examples 92-94

### Synthesis of (1S,2S)-2-(3-chlorophenyl)-N-(4-(((6-cyclopropyl-8-(3-oxo-2-azabicyclo[2.2.1]heptan-2-yl)imidazo[1,2-a]pyridin-2-yl)methyl)amino)pyridin-2-yl)cyclopropane-1-carboxamide (I-92)

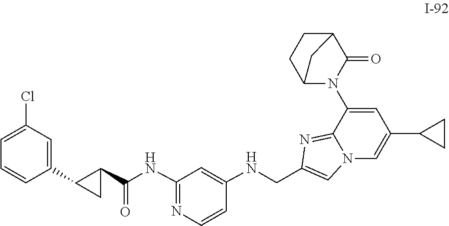 [SMILES: O=C(Nc1cc(NCc2cn3cc(C4CC4)cc(N4C(=O)C5CCC4C5)c3n2)ccn1)[C@H]1C[C@@H]1c1cccc(Cl)c1]

Using a similar procedure to that used for rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the title compound was prepared using (S,S)-2-(3-chlorophenyl)cyclopropane-1-carboxylic acid (23 mg, 0.12 mmol) and 2-(2-(((2-aminopyridin-4-yl)amino)methyl)-6-cyclopropylimidazo[1,2-a]pyridin-8-yl)-2-azabicyclo[2.2.1]heptan-3-one (50 mg, 0.13 mmol), then purified by preparative LCMS to give the title compound (21 mg, 32%) as a mixture of diastereomers. ESI-MS (M+H)+: 567.3, ¹H NMR (400 MHz, DMSO) δ 10.52-10.52 (m, 1H), 8.17 (d, J=1.1 Hz, 1H), 7.79 (d, J=6.0 Hz, 1H), 7.69 (s, 1H), 7.36-7.34 (m, 3H), 7.34-7.30 (m, 3H), 7.27 (d, J=5.5 Hz, 2H), 7.16 (d, J=7.7 Hz, 1H), 6.44 (d, J=2.3 Hz, 1H), 5.47 (s, 1H), 4.42 (d, J=5.5 Hz, 2H), 2.84 (d, J=2.4 Hz, 1H), 2.44-2.32 (m, 2H), 2.03 (d, J=9.4 Hz, 1H), 1.95-1.74 (m, 4H), 1.66-1.40 (m, 4H), 0.95-0.88 (m, 2H), 0.66-0.60 (m, 2H).

The mixture was separated using SFC (YMC Amylose-C 4.6×250 mm, 5 μm 50/50 IPA (0.1% DEA)/CO₂, 0.95 mL/min, 120 bar, 40° C.) to give two diastereomers: (1S,2S)-2-(3-chlorophenyl)-N-(4-(((6-cyclopropyl-8-((1R,4S)-3-oxo-2-azabicyclo[2.2.1]heptan-2-yl)imidazo[1,2-a]pyridin-2-yl)methyl)amino)pyridin-2-yl)cyclopropane-1-carboxamide and (1S,2S)-2-(3-chlorophenyl)-N-(4-(((6-cyclopropyl-8-((1S,4R)-3-oxo-2-azabicyclo[2.2.1]heptan-2-yl)imidazo[1,2-a]pyridin-2-yl)methyl)amino)pyridin-2-yl)cyclopropane-1-carboxamide.

First eluting isomer (I-93, 8.8 mg): ESI-MS (M+H)+: 567.2, ¹H NMR (400 MHz, DMSO) δ 10.52-10.52 (m, 1H), 8.17 (d, J=1.1 Hz, 1H), 7.79 (d, J=6.0 Hz, 1H), 7.69 (s, 1H), 7.36-7.34 (m, 3H), 7.34-7.30 (m, 3H), 7.27 (d, J=5.5 Hz, 2H), 7.16 (d, J=7.7 Hz, 1H), 6.44 (d, J=2.3 Hz, 1H), 5.47 (s, 1H), 4.42 (d, J=5.5 Hz, 2H), 2.84 (d, J=2.4 Hz, 1H), 2.44-2.32 (m, 2H), 2.03 (d, J=9.4 Hz, 1H), 1.95-1.74 (m, 4H), 1.66-1.40 (m, 4H), 0.95-0.88 (m, 2H), 0.66-0.60 (m, 2H). RT=2.02 min, 99.9% e.e.

Second eluting isomer (I-94, 7.9 mg): ESI-MS (M+H)+: 567.3, ¹H NMR (400 MHz, DMSO) δ 10.52-10.52 (m, 1H), 8.17 (d, J=1.1 Hz, 1H), 7.79 (d, J=6.0 Hz, 1H), 7.69 (s, 1H), 7.36-7.34 (m, 3H), 7.34-7.30 (m, 3H), 7.27 (d, J=5.5 Hz, 2H), 7.16 (d, J=7.7 Hz, 1H), 6.44 (d, J=2.3 Hz, 1H), 5.47 (s, 1H), 4.42 (d, J=5.5 Hz, 2H), 2.84 (d, J=2.4 Hz, 1H), 2.44-2.32 (m, 2H), 2.03 (d, J=9.4 Hz, 1H), 1.95-1.74 (m, 4H), 1.66-1.40 (m, 4H), 0.95-0.88 (m, 2H), 0.66-0.60 (m, 2H). RT=2.8 min, 99.6% e.e

### Examples 95-97

### Synthesis of (S,2S)-2-(3-chlorophenyl)-N-(4-(((6-cyclopropyl-8-(2-oxo-3-azabicyclo[3.1.0]hexan-3-yl)imidazo[1,2-a]pyridin-2-yl)methyl)amino)pyridin-2-yl)cyclopropane-1-carboxamide (I-95)

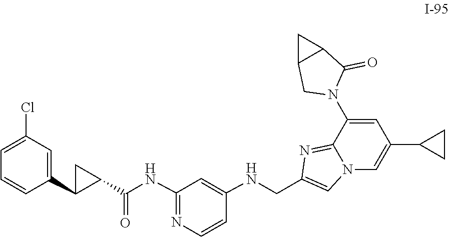 [SMILES: O=C(Nc1cc(NCc2cn3cc(C4CC4)cc(N4CC5CC5C4=O)c3n2)ccn1)[C@H]1C[C@@H]1c1cccc(Cl)c1]

Using a similar procedure to that used for rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the title compound was prepared using (S,S)-2-(3-chlorophenyl)cyclopropane-1-carboxylic acid (32 mg, 0.16 mmol) and 3-(2-(((2-aminopyridin-4-yl)amino)methyl)-6-cyclopropylimidazo[1,2-a]pyridin-8-yl)-3-azabicyclo[3.1.0]hexan-2-one (60 mg, 0.16 mmol), then purified by preparative LCMS to give the title compound (30 mg, 34%) as a mixture of diastereomers. ESI-MS (M+H)+: 553, ¹H NMR (400 MHz, DMSO): δ, ppm, 10.38 (1H, br s), 8.25 (1H, s), 7.80 (1H, d, J=6.4 Hz), 7.70 (1H, s), 7.36-7.25 (4H, m), 7.17 (1H, d, J=7.2 Hz), 7.06 (1H, s), 6.41 (1H, br s), 4.41-4.32 (3H, m), 4.00 (1H, dd, J=5.4, 10.0 Hz), 2.42 (1H, br s), 2.32 (1H, br s), 2.10 (1H, br s), 2.01 (1H, br s), 1.95-1.87 (1H, m), 1.49 (1H, br s), 1.41 (1H, br s), 1.27-1.18 (2H, m), 0.92 (2H, dd, J=2.0, 8.2 Hz), 0.86 (1H, br s), 0.66-0.62 (2H, m).

The mixture was separated using SFC (YMC Amylose-C 10×250 mm, 5 μm 55/45 MeOH (0.1% DEA)/CO₂, 15 mL/min, 120 bar, 40° C.) to give two diastereomers: (1S,2S)-2-(3-chlorophenyl)-N-(4-(((6-cyclopropyl-8-((1S,5R)-2-oxo-3-azabicyclo[3.1.0]hexan-3-yl)imidazo[1,2-a]pyridin-2-yl)methyl)amino)pyridin-2-yl)cyclopropane-1-carboxamide and (1S,2S)-2-(3-chlorophenyl)-N-(4-(((6-cyclopropyl-8-((1R,5S)-2-oxo-3-azabicyclo[3.1.0]hexan-3-yl)imidazo[1,2-a]pyridin-2-yl)methyl)amino)pyridin-2-yl)cyclopropane-1-carboxamide.

First eluting isomer (I-96, 12.68 mg): ESI-MS (M+H)+: 553, ¹H NMR (400 MHz, DMSO): δ, ppm, 10.35 (1H, s), 8.26 (1H, dd, J=0.5, 1.6 Hz), 7.79 (1H, d, J=6.0 Hz), 7.68 (1H, s), 7.49 (1H, s), 7.35 (1H, ddd, J=7.4, 7.4, 1.3 Hz), 7.30-7.27 (2H, m), 7.18 (1H, ddd, J=1.3, 1.3, 7.4 Hz), 7.15 (1H, d, J=6.0 Hz), 7.08 (1H, d, J=1.6 Hz), 6.36 (1H, dd, J=2.2, 5.8 Hz), 4.41-4.37 (3H, m), 4.03 (1H, dd, J=1.2, 10.3 Hz), 2.41-2.40 (2H, m), 2.14-2.14 (1H, m), 2.04-2.04 (1H, m), 1.97-1.89 (1H, m), 1.51-1.45 (1H, m), 1.41-1.35 (1H, m), 1.27-1.21 (1H, m), 0.96-0.92 (2H, m), 0.88 (1H, ddd, J=4.2, 4.2, 3.3 Hz), 0.69-0.65 (2H, m). RT=8.73 min, 99.9% e.e.

Second eluting isomer (I-97, 17.75 mg): ESI-MS (M+H)+: 553, ¹H NMR (400 MHz, DMSO): δ, ppm, 10.35 (1H, s), 8.26 (1H, dd, J=0.5, 1.6 Hz), 7.79 (1H, d, J=6.0 Hz), 7.68 (1H, s), 7.49 (1H, s), 7.35 (1H, ddd, J=7.4, 7.4, 1.3 Hz), 7.30-7.27 (2H, m), 7.18 (1H, ddd, J=1.3, 1.3, 7.4 Hz), 7.15 (1H, d, J=6.0 Hz), 7.08 (1H, d, J=1.6 Hz), 6.36 (1H, dd, J=2.2, 5.8 Hz), 4.41-4.37 (3H, m), 4.03 (1H, dd, J=1.2, 10.3 Hz), 2.41-2.40 (2H, m), 2.14-2.14 (1H, m), 2.04-2.04 (1H, m), 1.97-1.89 (1H, m), 1.51-1.45 (1H, m), 1.41-1.35 (1H, m), 1.27-1.21 (1H, m), 0.96-0.92 (2H, m), 0.88 (1H, ddd, J=4.2, 4.2, 3.3 Hz), 0.69-0.65 (2H, m). RT=12.64 min, 98% e.e.

### Example 98

### Synthesis of rac-(1S*,2S*)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-98)

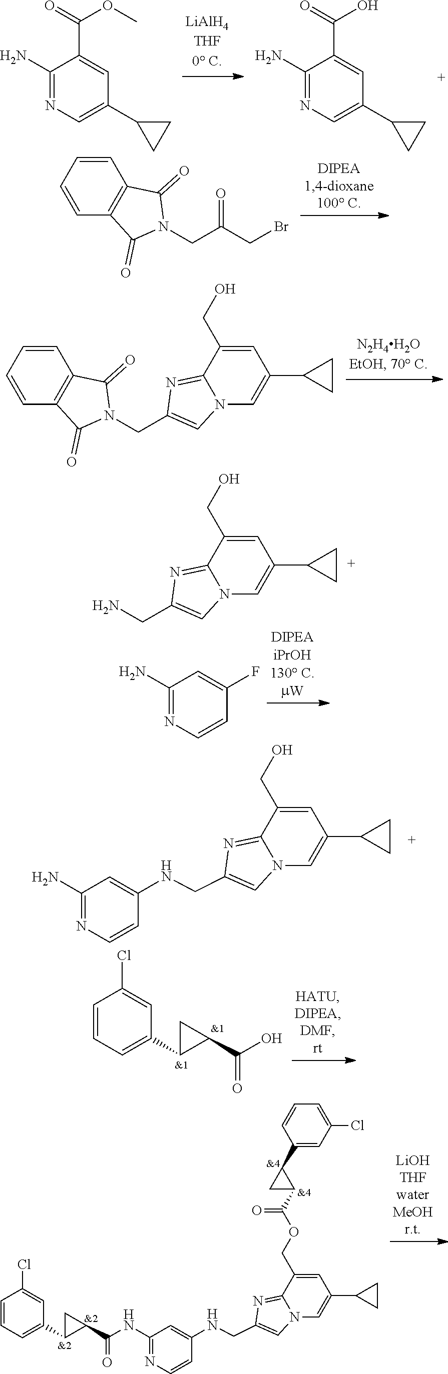

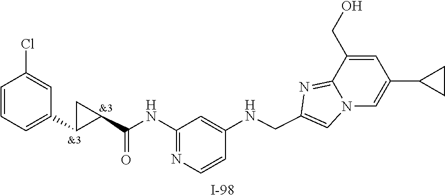

Synthesis of (2-amino-5-cyclopropylpyridin-3-yl)methanol. LiAlH₄ solution (1.0 M in THF, 12.5 mL, 12.5 mmol) was added dropwise to a stirred solution of methyl 2-amino-5-cyclopropylnicotinate (3.0 g, 16 mmol) in THF (60 mL) at 0° C. under a N₂ atmosphere. The mixture was stirred at 0° C. for 2 h, then further LiAlH₄ (1.0 M in THF, 2.5 mL, 2.5 mmol) was added dropwise. The mixture was stirred for 30 min, then water (0.57 mL) was added, followed by NaOH (20% aq·, 0.57 mL) and water (1.7 mL). The mixture was warmed to room temperature and diluted with Et₂O (60 mL). After 15 min MgSO₄ was added and the mixture was stirred for 75 min, then filtered through Celite® with Et₂O. The filtrate was concentrated in vacuo to give the title compound (2.5 g, 98%) as a yellow oil. ESI-MS (M+H)+: 311.2, ¹H NMR (400 MHz, DMSO) δ 7.75 (d, J=1.8 Hz, 1H), 7.12 (s, 1H), 5.47 (s, 2H), 5.18-5.13 (m, 1H), 4.36 (d, J=5.3 Hz, 2H), 1.85-1.77 (m, 1H), 0.91-0.84 (m, 2H), 0.60-0.54 (m, 2H).

Synthesis of 2-((6-cyclopropyl-8-(hydroxymethyl)imidazo[1,2-a]pyridin-2-yl)methyl)isoindoline-1,3-dione. A mixture of (2-amino-5-cyclopropylpyridin-3-yl)methanol (0.50 g, 3.0 mmol), 2-(3-bromo-2-oxopropyl)isoindoline-1,3-dione (1.3 g, 3.0 mmol), and DIPEA (0.53 mL, 3.0 mmol) in 1,4-dioxane (10 mL) was stirred at 100° C. for 2 h, then cooled to room temperature and allowed to stand for 18 h. The mixture was concentrated in vacuo, and the residue was purified by column chromatography on silica gel, eluting with 1-10% MeOH in DCM to give the title compound (0.50 g, 47%) as an orange gum. ESI-MS (M+H)+: 348.2, ¹H NMR (400 MHz, DMSO) δ 8.15 (s, 1H), 7.95-7.87 (m, 4H), 7.73 (s, 1H), 6.98 (d, J=1.3 Hz, 1H), 5.32-5.28 (m, 1H), 4.87 (s, 2H), 4.73 (d, J=5.8 Hz, 2H), 1.98-1.89 (m, 1H), 0.95-0.89 (m, 2H), 0.67-0.61 (m, 2H).

Synthesis of (2-(aminomethyl)-6-cyclopropylimidazo[1,2-a]pyridin-8-yl)methanol. A mixture of 2-((6-cyclopropyl-8-(hydroxymethyl)imidazo[1,2-a]pyridin-2-yl)methyl)isoindoline-1,3-dione (0.50 g, 1.4 mmol) and hydrazine monohydrate (0.18 mL, 2.9 mmol) in EtOH (14 mL) was stirred at 70° C. for 18 h. The mixture was loaded onto an SCX cartridge and washed with MeOH. The product was eluted with 7 N NH₃ in MeOH to give the title compound (0.25 g, 80%) as an orange gum. ESI-MS (M+H)+: 218.2, ¹H NMR (400 MHz, DMSO) δ 8.21 (s, 1H), 7.65 (s, 1H), 6.95 (d, J=1.4 Hz, 1H), 4.77 (s, 2H), 3.79 (s, 2H), 3.18 (s, 1H), 1.99-1.90 (m, 1H), 0.93 (ddd, J=4.3, 6.3, 8.3 Hz, 2H), 0.69-0.64 (m, 2H). NH₂ not observed

Synthesis of (2-(((2-aminopyridin-4-yl)amino)methyl)-6-cyclopropylimidazo[1,2-a]pyridin-8-yl)methanol. A mixture of (2-(aminomethyl)-6-cyclopropylimidazo[1,2-a]pyridin-8-yl)methanol (0.20 g, 0.92 mmol), 4-fluoropyridin-2-amine (0.10 g, 0.92 mmol), and DIPEA (0.24 mL, 1.4 mmol) in iPrOH (2.0 mL) was stirred at 130° C. in a microwave for 2 h. The mixture was concentrated in vacuo and the residue was purified by column chromatography on silica gel, eluting with a gradient of 1-50% (7 N NH₃ in MeOH) in DCM to give the title compound (0.14 g, 50%) as an orange gum. ESI-MS (M+H)+: 310.2, ¹H NMR (400 MHz, DMSO) δ 8.25 (s, 1H), 7.65 (s, 1H), 7.50 (d, J=5.8 Hz, 1H), 7.03 (d, J=1.5 Hz, 1H), 6.64-6.59 (m, 1H), 5.96 (dd, J=2.1, 5.9 Hz, 1H), 5.65 (d, J=2.0 Hz, 1H), 5.38-5.32 (m, 3H), 4.83 (d, J=5.3 Hz, 2H), 4.34 (d, J=5.8 Hz, 2H), 2.02-1.93 (m, 1H), 0.99-0.93 (m, 2H), 0.72-0.67 (m, 2H).

Synthesis of (2-(((2-((rac-(1S*,2S*))-2-(3-chlorophenyl)cyclopropane-1-carboxamido)pyridin-4-yl)amino)methyl)-6-cyclopropylimidazo[1,2-a]pyridin-8-yl)methyl (rac-(1S*,2S*))-2-(3-chlorophenyl)cyclopropane-1-carboxylate. A mixture of (2-(((2-aminopyridin-4-yl)amino)methyl)-6-cyclopropylimidazo[1,2-a]pyridin-8-yl)methanol (0.036 g, 0.12 mmol), rac-(1S*,2S*)-2-(3-chlorophenyl)cyclopropane-1-carboxylic acid (0.023 g, 0.12 mmol), HATU (0.049 g, 0.13 mmol), and DIPEA (0.041 mL, 0.23 mmol) in DMF (1.0 mL) was stirred at room temperature for 18 h. Water (10 mL) and brine (sat·aq·, 15 mL) were added, and the mixture was extracted with EtOAc (3×25 mL). The combined organics were dried (MgSO₄), filtered, and concentrated in vacuo to give the title compound (0.070 g, 91%), which was used directly in the next step without further purification.]

Synthesis of rac-(1S*,2S*)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-98). A mixture of (2-(((2-((rac-(1S*,2S*))-2-(3-chlorophenyl)cyclopropane-1-carboxamido)pyridin-4-yl)amino)methyl)-6-cyclopropylimidazo[1,2-a]pyridin-8-yl)methyl (rac-(1S*,2S*))-2-(3-chlorophenyl)cyclopropane-1-carboxylate (0.045 g, 0.068 mmol) and LiOH (1.0 M aq·, 0.034 mL, 0.034 mmol) in THF (3.0 mL) was stirred at room temperature for 3 h. MeOH (0.5 mL) was added and the mixture was stirred at room temperature for 30 min. Further LiOH (1.0 M aq·, 0.10 mL, 0.10 mmol) was added and the mixture was stirred at room temperature for 1 h. The mixture was concentrated in vacuo, then dissolved in brine (sat·aq·, 25 mL) and water (25 mL). The mixture was extracted with EtOAc (3×50 mL) and the combined organic layers were dried (MgSO₄), filtered, and concentrated in vacuo. The residue was purified by preparative HPLC to give the title compound (2.3 mg, 7%) as a white solid (formic acid salt). ESI-MS (M+H)+: 488.2, ¹H NMR (400 MHz, DMSO) δ 10.31 (s, 1H), 8.27-8.26 (m, 1H), 8.20 (s, 1H), 7.75 (d, J=5.7 Hz, 1H), 7.62 (s, 1H), 7.45-7.42 (m, 1H), 7.34-7.28 (m, 1H), 7.26-7.23 (m, 2H), 7.16-7.09 (m, 2H), 6.99-6.97 (m, 1H), 6.31 (dd, J=2.1, 5.8 Hz, 1H), 5.31 (br s, 1H), 4.78 (s, 2H), 4.35 (d, J=5.7 Hz, 2H), 2.40-2.31 (m, 2H), 1.96-1.88 (m, 1H), 1.47-1.41 (m, 1H), 1.37-1.31 (m, 1H), 0.94-0.88 (m, 2H), 0.67-0.62 (m, 2H).

### Synthesis of N⁴-((8-(((tert-butyldimethylsilyl)oxy)methyl)-6-cyclopropylimidazo[1,2-a]pyridin-2-yl)methyl)pyridine-2,4-diamine

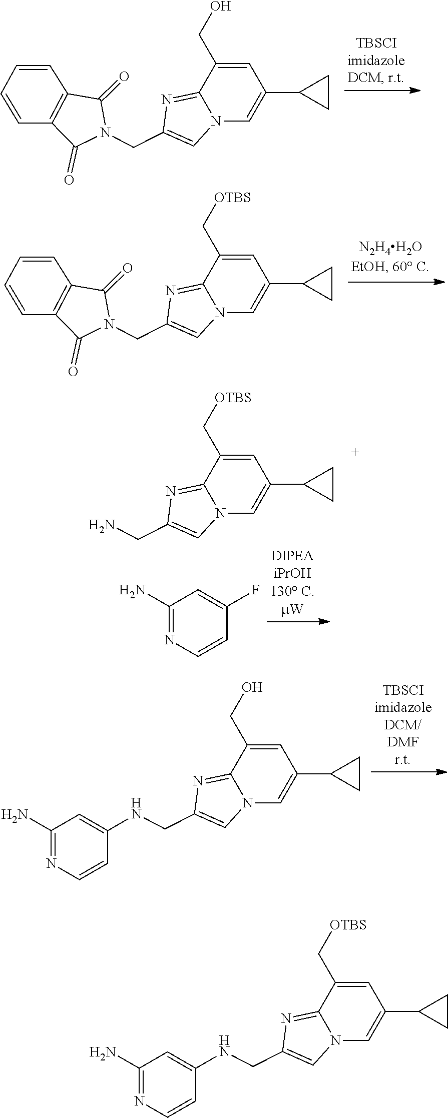 [SMILES: O=C1c2ccccc2C(=O)N1Cc1cn2cc(C3CC3)cc(CO)c2n1 ; CC(C)(C)[Si](C)(C)OCc1cc(C2CC2)cn2cc(CN3C(=O)c4ccccc4C3=O)nc12 ; CC(C)(C)[Si](C)(C)OCc1cc(C2CC2)cn2cc(CN)nc12 ; Nc1cc(F)ccn1 ; Nc1cc(NCc2cn3cc(C4CC4)cc(CO)c3n2)ccn1 ; CC(C)(C)[Si](C)(C)OCc1cc(C2CC2)cn2cc(CNc3ccnc(N)c3)nc12]

Synthesis of 2-((8-(((tert-butyldimethylsilyl)oxy)methyl)-6-cyclopropylimidazo[1,2-a]pyridin-2-yl)methyl)isoindoline-1,3-dione. TBDMS-Cl (0.65 g, 4.3 mmol) and imidazole (0.49 g, 7.2 mmol) were added to a solution of 2-((6-cyclopropyl-8-(hydroxymethyl)imidazo[1,2-a]pyridin-2-yl)methyl)isoindoline-1,3-dione (1.3 g, 3.6 mmol) in DCM (30 mL). The mixture was stirred at room temperature for 18 h. Water (100 mL) was added and the mixture was extracted with DCM (3×100 ml). The combined organic layers were dried over MgSO₄, filtered, and concentrated in vacuo. The residue was purified by column chromatography on silica gel, eluting with a gradient of 20-80% EtOAc in cyclohexane to give the title compound (0.89 g, 54%) as an orange solid. ESI-MS (M+H)+: 462.4, ¹H NMR (400 MHz, DMSO) δ 8.22 (s, 1H), 7.97-7.89 (m, 4H), 7.78 (s, 1H), 6.95 (d, J=1.1 Hz, 1H), 4.94 (s, 2H), 4.89 (s, 2H), 2.01-1.93 (m, 1H), 0.99-0.94 (m, 2H), 0.92 (s, 9H), 0.67-0.61 (m, 2H), 0.10 (s, 6H).

Synthesis of (8-(((tert-butyldimethylsilyl)oxy)methyl)-6-cyclopropylimidazo[1,2-a]pyridin-2-yl)methanamine. A mixture of 2-((8-(((tert-butyldimethylsilyl)oxy)methyl)-6-cyclopropylimidazo[1,2-a]pyridin-2-yl)methyl)isoindoline-1,3-dione (0.89 g, 1.9 mmol) and hydrazine monohydrate (0.24 mL, 3.9 mmol) in CHCl₃ (20 mL) was stirred at 60° C. for 18 h. The mixture was diluted with EtOAc and filtered. The filtrate was concentrated in vacuo to give the title compound (0.82 g, quantitative) as an orange gum. ¹H NMR (400 MHz, DMSO) δ 8.27 (s, 1H), 7.70 (s, 1H), 6.92 (d, J=1.5 Hz, 1H), 4.99 (s, 2H), 3.83 (s, 2H), 2.02-1.94 (m, 1H), 1.01-0.95 (m, 11H), 0.69-0.63 (m, 2H), 0.15 (s, 6H). NH₂ not observed.

Synthesis of (2-(((2-aminopyridin-4-yl)amino)methyl)-6-cyclopropylimidazo[1,2-a]pyridin-8-yl)methanol A mixture of (8-(((tert-butyldimethylsilyl)oxy)methyl)-6-cyclopropylimidazo[1,2-a]pyridin-2-yl)methanamine (0.41 g, 1.2 mmol), 4-fluoropyridin-2-amine (0.14 g, 1.2 mmol), and DIPEA (0.65 mL, 3.7 mmol) in iPrOH (4.0 mL) was stirred at 130° C. in a microwave for 1.5 h. The mixture was then stirred at 130° C. in a microwave for a further 4 h. The mixture was concentrated in vacuo and the residue was purified by column chromatography on silica gel, eluting with 0-100% 7 N NH₃ in MeOH in DCM to give the title compound (0.11 g, 21%) as a yellow gum. ESI-MS (M+H)+: 310, ¹H NMR (400 MHz, DMSO) δ 8.21 (s, 1H), 8.09 (dd, J=3.4, 5.9 Hz, 1H), 7.89 (dd, J=3.3, 6.0 Hz, 1H), 7.62 (s, 1H), 7.46 (d, J=6.0 Hz, 1H), 6.99 (d, J=1.4 Hz, 1H), 6.73-6.68 (m, 1H), 5.94 (dd, J=2.1, 6.0 Hz, 1H), 5.62 (d, J=2.0 Hz, 1H), 5.46 (s, 2H), 4.79 (s, 2H), 4.31 (d, J=5.8 Hz, 2H), 3.18 (s, 1H), 1.98-1.90 (m, 1H), 0.96-0.89 (m, 2H), 0.68-0.63 (m, 2H).

Synthesis of N⁴-((8-(((tert-butyldimethylsilyl)oxy)methyl)-6-cyclopropylimidazo[1,2-a]pyridin-2-yl)methyl)pyridine-2,4-diamine. TBDMS-Cl (0.22 g, 1.5 mmol) and imidazole (0.10 g, 1.5 mmol) were added to a solution of (2-(((2-aminopyridin-4-yl)amino)methyl)-6-cyclopropylimidazo[1,2-a]pyridin-8-yl)methanol (0.15 g, 0.49 mmol) in DCM (10 mL) and DMF (5 mL). The mixture was stirred at room temperature for 18 h. Water (50 mL) and NaHCO₃ (sat·aq·, 50 mL) were added, and the mixture was extracted with DCM (3×50 ml). The combined organic layers were dried over MgSO₄, filtered, and concentrated in vacuo. The residue was purified by column chromatography on silica gel, eluting with 0-10% methanolic ammonia in DCM to give the title compound (0.078 g, 37%) as a yellow oil. ¹H NMR (400 MHz, DMSO) δ 8.27 (s, 1H), 7.65 (s, 1H), 7.48 (d, J=5.9 Hz, 1H), 6.95 (d, J=1.5 Hz, 1H), 6.61-6.56 (m, 1H), 5.93 (dd, J=2.1, 5.8 Hz, 1H), 5.63 (d, J=2.0 Hz, 1H), 5.30 (s, 2H), 5.01 (s, 2H), 4.32 (d, J=5.8 Hz, 2H), 2.01-1.93 (m, 1H), 0.98 (s, 11H), 0.67-0.62 (m, 2H), 0.16 (s, 6H).

### Example 99

### Synthesis of (1S,2S)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-99)

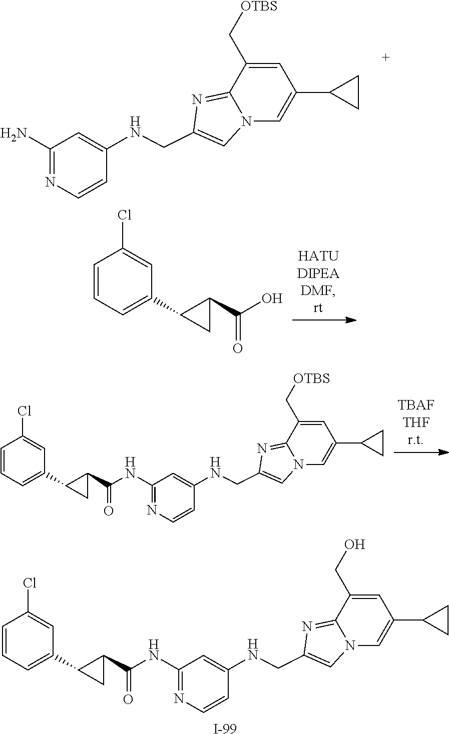

Synthesis of (1S,2S)—N-(4-(((8-(((tert-butyldimethylsilyl)oxy)methyl)-6-cyclopropylimidazo[1,2-a]pyridin-2-yl)methyl)amino)pyridin-2-yl)-2-(3-chlorophenyl)cyclopropane-1-carboxamide. A mixture of N⁴-((8-(((tert-butyldimethylsilyl)oxy)methyl)-6-cyclopropylimidazo[1,2-a]pyridin-2-yl)methyl)pyridine-2,4-diamine (0.11 g, 0.26 mmol), (1S,2S)-2-(3-chlorophenyl)cyclopropane-1-carboxylic acid (0.050 g, 0.26 mmol), HATU (0.11 g, 0.28 mmol), and DIPEA (0.089 mL, 0.51 mmol) in DMF (3.0 mL) was stirred at room temperature for 18 h. Water (50 mL) and NaHCO₃ (sat·aq·, 50 mL) were added and the mixture was extracted with DCM (3×50 mL). The combined organics were dried over MgSO₄, filtered, and concentrated in vacuo. The residue was purified by column chromatography on silica gel, eluting with 0-20% 7 N NH₃ in MeOH in DCM to give the title compound (0.076 g, 51%) as an orange oil, which was used directly in the next step without further purification.

Synthesis of (1S,2S)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-99). A mixture of (1S,2S)—N-(4-(((8-(((tert-butyldimethylsilyl)oxy)methyl)-6-cyclopropylimidazo[1,2-a]pyridin-2-yl)methyl)amino)pyridin-2-yl)-2-(3-chlorophenyl)cyclopropane-1-carboxamide (0.076 g, 0.13 mmol), and TBAF (1.0 M in THF, 0.15 mL, 0.15 mmol) in THF (3 mL) was stirred at room temperature for 3 h. Water (20 mL) and brine (sat·aq·, 5 mL) were added, and the mixture was extracted with EtOAc (3×25 mL). The combined organics were dried over MgSO₄, filtered, and concentrated in vacuo. The residue was purified by column chromatography on silica gel, eluting with 0-10% 7 N NH₃ in MeOH in DCM to give the title compound (0.011 g, 17%) as a yellow solid. ESI-MS (M+H)+. 488.3, ¹H NMR (400 MHz, DMSO) δ 10.31 (s, 1H), 8.21-8.20 (m, 1H), 7.75 (d, J=5.7 Hz, 1H), 7.62 (s, 1H), 7.43 (br s, 1H), 7.32-7.23 (m, 3H), 7.16-7.11 (m, 2H), 6.99-6.97 (m, 1H), 6.31 (dd, J=2.1, 5.7 Hz, 1H), 5.31 (t, J=5.8 Hz, 1H), 4.78 (d, J=5.6 Hz, 2H), 4.35 (d, J=5.7 Hz, 2H), 2.41-2.31 (m, 2H), 1.96-1.88 (m, 1H), 1.47-1.41 (m, 1H), 1.38-1.31 (m, 1H), 0.94-0.87 (m, 2H), 0.67-0.62 (m, 2H).

### Example 100

### Synthesis of rac-(1S*,2S*)-2-(5-chloro-2-cyanophenyl)-N-(4-(((6-cyclopropyl-8-(hydroxymethyl)imidazo[1,2-a]pyridin-2-yl)methyl)amino)pyridin-2-yl)cyclopropane-1-carboxamide (I-100)

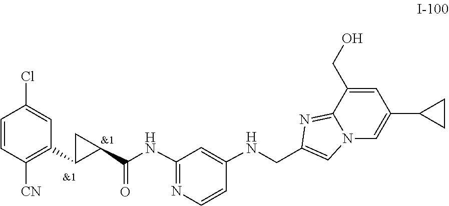 [SMILES: N#Cc1ccc(Cl)cc1[C@@H]1C[C@H]1C(=O)Nc1cc(NCc2cn3cc(C4CC4)cc(CO)c3n2)ccn1]

Using a similar procedure to that used for (1S,2S)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide, rac-(1S*,2S*)-2-(5-chloro-2-cyanophenyl)-N-(4-(((6-cyclopropyl-8-(hydroxymethyl)imidazo[1,2-a]pyridin-2-yl)methyl)amino)pyridin-2-yl)cyclopropane-1-carboxamide was prepared from rac-(1S*,2S*)-(5-chloro-2-cyanophenyl)cyclopropane-1-carboxylic acid and N⁴-((8-(((tert-butyldimethylsilyl)oxy)methyl)-6-cyclopropylimidazo[1,2-a]pyridin-2-yl)methyl)pyridine-2,4-diamine (3.3 mg, 6%). ESI-MS (M+H)+: 513.3, ¹H NMR (400 MHz, DMSO) δ 10.47-10.47 (m, 1H), 8.21 (s, 1H), 7.86 (d, J=8.4 Hz, 1H), 7.79-7.76 (m, 1H), 7.66 (s, 1H), 7.55-7.50 (m, 1H), 7.41 (s, 1H), 6.99 (s, 1H), 5.35-5.31 (m, 1H), 4.78 (d, J=5.3 Hz, 2H), 4.40-4.39 (m, 2H), 1.95-1.89 (m, 1H), 1.57-1.57 (m, 2H), 0.96-0.88 (m, 2H), 0.66-0.64 (m, 2H). Two cyclopropyl CH signals obscured by DMSO signal. Three signals in aromatic region missing and/or extremely broad.

### Synthesis of N⁵-((6-cyclopropylimidazo[1,2-a]pyridin-2-yl)methyl)pyridazine-3,5-diamine

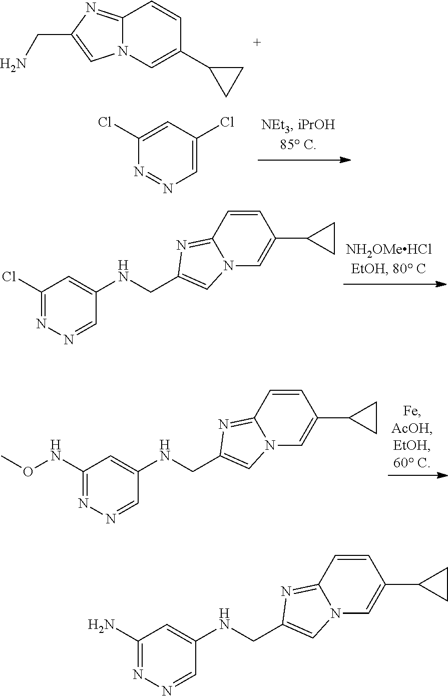

Synthesis of 6-chloro-N-((6-cyclopropylimidazo[1,2-a]pyridin-2-yl)methyl)pyridazin-4-amine. Triethylamine (0.56 mL, 4.0 mmol) was added to a stirred mixture of (6-cyclopropylimidazo[1,2-a]pyridin-2-yl)methanamine (300 mg, 1.3 mmol) and 3,5-dichloropyridazine (240 mg, 1.6 mmol) in iPrOH (3.0 mL). The reaction was then stirred at 85° C. for 18 h. The reaction was cooled to room temperature and diluted with EtOAc (30 mL) and water (30 mL). The layers were separated, and the aqueous layer was further extracted with EtOAc (2×20 mL). The organic layers were combined, dried over MgSO₄ and concentrated in vacuo. The residue was purified by column chromatography on silica gel, eluting with a gradient of 0-5% MeOH in DCM to give the title compound (0.16 g, 41%). ESI-MS (M+H)+: 300.0, 302.0, ¹H NMR (400 MHz, DMSO) δ 8.65 (s, 1H), 8.34 (t, J=1.3 Hz, 1H), 7.93 (s, 1H), 7.77 (s, 1H), 7.42 (d, J=10.4 Hz, 1H), 7.01 (dd, J=1.8, 9.2 Hz, 1H), 6.83 (d, J=2.3 Hz, 1H), 4.47 (d, J=6.0 Hz, 2H), 1.98-1.90 (m, 1H), 0.96-0.91 (m, 2H), 0.71-0.67 (m, 2H).

Synthesis of N-((6-cyclopropylimidazo[1,2-a]pyridin-2-yl)methyl)-6-(methoxyamino)pyridazin-4-amine. O-Methyl hydroxylamine hydrochloride (690 mg, 8.2 mmol) was added to a stirred mixture of 6-chloro-N-((6-cyclopropylimidazo[1,2-a]pyridin-2-yl)methyl)pyridazin-4-amine (160 mg, 0.55 mmol) in ethanol (9.0 mL), and the reaction was stirred at 80° C. for 24 h. The reaction was cooled to room temperature and filtered through Celite®. The filtrate was concentrated in vacuo. The residue was suspended in NaHCO₃ (sat·aq·, 30 mL) and extracted with DCM (3×30 mL). The organic layers were combined, dried over MgSO₄, and concentrated in vacuo to give the title compound as a crude oil (183 mg), which was carried forward to the next step without further purification. ESI-MS (M+H)+: 311.2.

Synthesis of N⁵-((6-cyclopropylimidazo[1,2-a]pyridin-2-yl)methyl)pyridazine-3,5-diamine. Iron powder (170 mg, 3.0 mmol) and acetic acid (20% aq·, 2.8 mL, 9.8 mmol) were added to a stirred solution of N-((6-cyclopropylimidazo[1,2-a]pyridin-2-yl)methyl)-6-(methoxyamino)pyridazin-4-amine (180 mg, 0.59 mol) in ethanol (19 mL). The reaction was heated at 60° C. for 2.5 h. The reaction was cooled to room temperature, diluted with EtOAc (50 mL), and filtered through Celite®. The filtrate was concentrated in vacuo, then the residue was suspended in DCM/MeOH (1:1, 10 mL) and filtered. The filtrate was concentrated in vacuo. The residue was purified by column chromatography on silica gel, eluting with a gradient of 0-5% (7N NH₃ in MeOH) in DCM to give the title compound (0.097 g, 59%). ESI-MS (M+H)+: 281.2, ¹H NMR (400 MHz, DMSO) δ 8.24 (s, 1H), 7.94 (d, J=2.3 Hz, 1H), 7.57 (s, 1H), 7.31 (d, J=9.3 Hz, 1H), 6.99 (dd, J=5.3, 5.3 Hz, 1H), 6.91 (dd, J=1.3, 9.3 Hz, 1H), 5.65 (s, 3H), 4.25 (d, J=5.6 Hz, 2H), 1.89-1.80 (m, 1H), 0.88-0.81 (m, 2H), 0.63-0.56 (m, 2H).

Using a similar procedure to that used for rac-(1S*,2S*)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide, the compounds in Table 12 were prepared using N⁵-((6-cyclopropylimidazo[1,2-a]pyridin-2-yl)methyl)pyridazine-3,5-diamine and an appropriate acid and purified by preparative LCMS.

TABLE 12

| | | Coupling Partner/ |
|---|---|---|
| Example | Compound | Analytical Data |
| Example 101 | 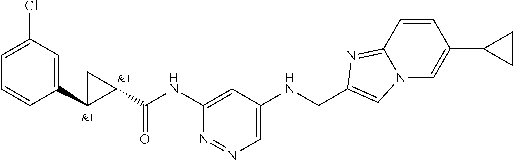 [SMILES: O=C(Nc1cc(NCc2cn3cc(C4CC4)ccc3n2)cnn1)[C@H]1C[C@@H]1c1cccc(Cl)c1] rac-(1S*,2S*)-2-(3-chlorophenyl)-N-(5- (((6-cyclopropylimidazo[1,2-a]pyridin-2- yl)methyl)amino)pyridazin-3- yl)cyclopropane-1-carboxamide (I-101) | rac-(1S*,2S*)-2-(3- chlorophenyl)cyclopropane-1- carboxylic acid ESI-MS (M + H)+: 459.2, ¹H NMR (400 MHz, DMSO) δ 10.86 (s, 1H), 8.44 (d, J = 2.5 Hz, 1H), 8.33 (s, 1H), 7.70 (s, 1H), 7.56 (dd, J = 5.8, 5.8 Hz, 1H), 7.50 (d, J = 1.9 Hz, 1H), 7.41 (d, J = 9.4 Hz, 1H), 7.36-7.30 (m, 1H), 7.26 (dd, J = 1.6, 3.5 Hz, 2H), 7.16 (d, J = 7.7 Hz, 1H), 7.00 (dd, J = 1.7, 9.3 Hz, 1H), 4.41 (d, J = 5.6 Hz, 2H), 2.41 (dd, J = 6.5, 6.5 Hz, 2H), 2.01- 1.89 (m, 1H), 1.52-1.37 (m, 2H), 0.96-0.89 (m, 2H), 0.71-0.65 (m, |
| | | 2H). |
| Example 102 | 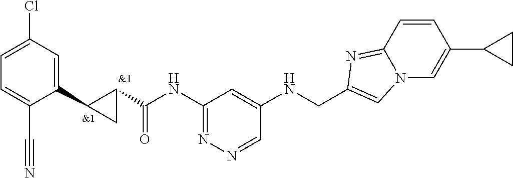 [SMILES: N#Cc1ccc(Cl)cc1[C@@H]1C[C@H]1C(=O)Nc1cc(NCc2cn3cc(C4CC4)ccc3n2)cnn1] rac-(1S*,2S*)-2-(5-chloro-2-cyanophenyl)- N-(5-(((6-cyclopropylimidazo[1,2- a]pyridin-2-yl)methyl)amino)pyridazin-3- yl)cyclopropane-1-carboxamide (I-102) | rac-(1S*,2S*)-2-(5-chloro-2- cyanophenyl)cyclopropane-1- carboxylic acid ESI-MS (M + H )+: 484.3, ¹H NMR (400 MHz, DMSO) δ 10.91 (s, 1H), 8.46-8.43 (m, 1H), 8.33 (s, 1H), 7.87 (d, J = 8.6 Hz, 1H), 7.71 (s, 1H), 7.57 (s, 1H), 7.53 (d, J = 9.9 Hz, 2H), 7.41 (d, J = 9.1 Hz, 2H), 7.00 (d, J = 9.1 Hz, 1H), 4.42 (d, J = 4.5 Hz, 2H), 2.60-2.55 (m, 2H), 1.94-1.89 (m, 1H), 1.62-1.55 (m, 2H), 0.93 (d, J = 7.1 Hz, 2H), 0.68 (d, J = 4.3 Hz, 2H). |

### Example 103

### Synthesis of rac-(1S*,2S*)-2-(3-chlorophenyl)-N-(5-((6-cyclopropylimidazo[1,2-a]pyridin-2-yl)methoxy)pyridazin-3-yl)cyclopropane-1-carboxamide (I-103)

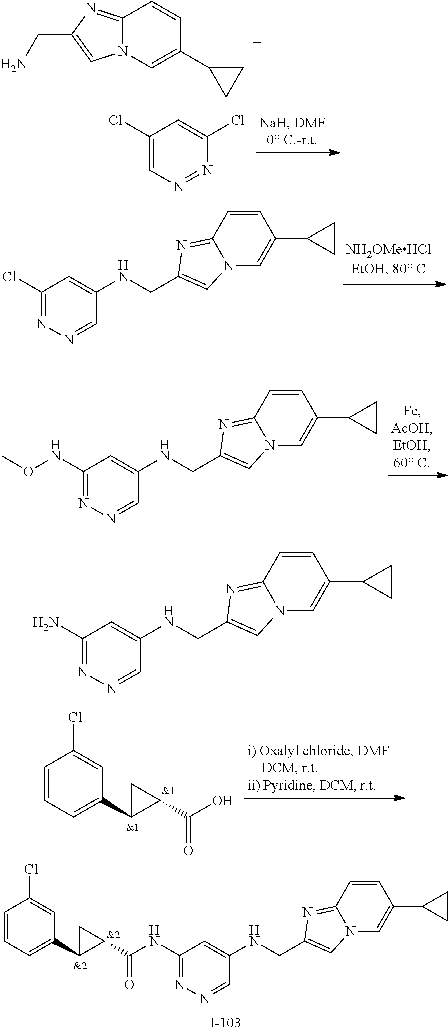

Synthesis of 2-(((6-chloropyridazin-4-yl)oxy)methyl)-6-cyclopropylimidazo[1,2-a]pyridine. (6-cyclopropylimidazo[1,2-a]pyridin-2-yl)methanol (150 mg, 0.80 mmol) in DMF (1.0 mL) was added to a stirred mixture of NaH (60% in mineral oil, 54 mg, 1.4 mmol) in DMF (1.0 mL) at 0° C. and stirred at 0° C. for 2 h. 3,5-Dichloropyridazine (140 mg, 0.96 mmol) in DMF (1.0 mL) was then added and the reaction was allowed to warm to room temperature and stirred for 18 h. The reaction was quenched with water (10 mL) and extracted with DCM (3×15 mL). The organic layers were combined, dried over MgSO₄, and concentrated in vacuo. The residue was purified by column chromatography on silica gel, eluting with a gradient of 0-100% EtOAc in DCM to give the title compound (0.17 g, 70%). ESI-MS (M+H)+: 301.0, ¹H NMR (400 MHz, CDCl₃) δ 8.91 (d, J=2.5 Hz, 1H), 7.89 (s, 1H), 7.57 (s, 1H), 7.49 (d, J=9.3 Hz, 1H), 7.18 (d, J=2.5 Hz, 1H), 6.99 (dd, J=1.5, 9.3 Hz, 1H), 5.34 (s, 2H), 1.94-1.86 (m, 1H), 1.02-0.95 (m, 2H), 0.72-0.65 (m, 2H).

Synthesis of N-(5-((6-cyclopropylimidazo[1,2-a]pyridin-2-yl)methoxy)pyridazin-3-yl)-O-methylhydroxylamine. O-Methyl hydroxylamine hydrochloride (830 mg, 10 mmol) was added to a stirred mixture of 2-(((6-chloropyridazin-4-yl)oxy)methyl)-6-cyclopropylimidazo[1,2-a]pyridine (200 mg, 0.67 mmol) in EtOH (25.0 mL) and the reaction was stirred at 80° C. for 24 h. The reaction was cooled to room temperature and filtered through Celite®. The filtrate was concentrated in vacuo, and the residue was suspended in NaHCO₃ (sat·aq·, 30 mL) and extracted with DCM (3×30 mL). The organic layers were combined, dried over MgSO₄, and concentrated in vacuo. The residue was purified by column chromatography on silica gel, eluting with a gradient of 0-10% MeOH in DCM to give the title compound (0.11 g, 53%). ESI-MS (M+H)+: 312, ¹H NMR (400 MHz, CDCl₃) δ 9.29 (s, 1H), 7.88 (s, 1H), 7.54 (s, 1H), 7.47 (d, J=9.3 Hz, 1H), 7.16 (s, 1H), 6.97 (dd, J=1.6, 9.0 Hz, 1H), 5.93 (s, 1H), 5.08 (s, 2H), 3.80 (s, 3H), 1.94-1.86 (m, 1H), 1.01-0.95 (m, 2H), 0.72-0.66 (m, 2H).

Synthesis of 5-((6-cyclopropylimidazo[1,2-a]pyridin-2-yl)methoxy)pyridazin-3-amine. Iron powder (110 mg, 1.9 mmol) and acetic acid (20% aq·, 0.9 mL) were added to a stirred solution of N-(5-((6-cyclopropylimidazo[1,2-a]pyridin-2-yl)methoxy)pyridazin-3-yl)-O-methylhydroxylamine (120 mg, 0.38 mol) in EtOH (6.0 mL). The reaction was heated at 60° C. for 1 h. The reaction was cooled to room temperature, diluted with EtOAc (50 mL), and filtered through Celite®. The filtrate was concentrated in vacuo, and the residue was suspended in DCM/MeOH (1:1, 10 mL) and filtered. The filtrate was concentrated in vacuo. The residue was purified by column chromatography on silica gel, eluting with a gradient of 0-5% (7 N NH₃ in MeOH) in DCM to give the title compound (0.10 g, 95%) which was carried through to the next step without further purification. ESI-MS (M+H)+: 282.1.

Synthesis of rac-(1S*,2S*)-2-(3-chlorophenyl)-N-(5-((6-cyclopropylimidazo[1,2-a]pyridin-2-yl)methoxy)pyridazin-3-yl)cyclopropane-1-carboxamide (I-103). Oxalyl chloride (48 μL, 0.55 mmol) was added to a stirred solution of rac-(1S*,2S*)-2-(3-chlorophenyl)cyclopropane-1-carboxylic acid (36 mg, 0.18 mmol) and DMF (3.5 μL, 0.046 mmol) in DCM (1.0 mL) and stirred at room temperature for 20 min. The reaction was concentrated in vacuo. The residue was dissolved in DCM (2.0 mL) and added to a stirred solution of 5-((6-cyclopropylimidazo[1,2-a]pyridin-2-yl)methoxy)pyridazin-3-amine (51 mg, 0.18 mmol) in DCM (1.0 mL). Pyridine (30 μL, 0.37 mmol) was then added and the reaction was stirred at room temperature for 18 h. The reaction was quenched with water (20 mL) and extracted with DCM (3×15 mL). The organic layers were combined, dried over MgSO₄, and concentrated in vacuo. The residue was purified by reverse phase preparative HPLC to give the title compound (4.1 mg, 5%). ESI-MS (M+H)+: 460.3, ¹H NMR (400 MHz, DMSO) δ 11.36 (s, 1H), 8.83 (d, J=2.8 Hz, 1H), 8.38 (s, 1H), 8.08 (d, J=2.8 Hz, 1H), 7.94 (s, 1H), 7.46 (d, J=9.5 Hz, 1H), 7.37-7.27 (m, 3H), 7.19 (d, J=7.6 Hz, 1H), 7.04 (dd, J=1.6, 9.4 Hz, 1H), 5.35 (s, 2H), 2.49-2.42 (m, 2H), 2.00-1.92 (m, 1H), 1.58-1.46 (m, 2H), 0.98-0.91 (m, 2H), 0.73-0.69 (m, 2H).

### Example 104

### Synthesis of rac-(1S*,2S*)-2-(3-chlorophenyl)-N-(6-((6-cyclopropylimidazo[1,2-a]pyridin-2-yl)methoxy)pyrimidin-4-yl)cyclopropane-1-carboxamide (I-104)

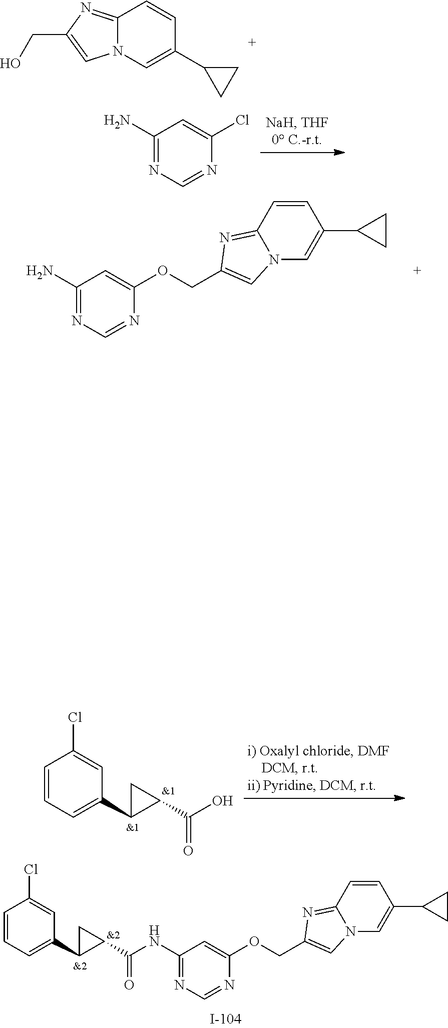

Synthesis of 6-((6-cyclopropylimidazo[1,2-a]pyridin-2-yl)methoxy)pyrimidin-4-amine. (6-Cyclopropylimidazo[1,2-a]pyridin-2-yl)methanol (50 mg, 0.27 mmol) in THF (1.0 mL) was added to a stirred mixture of NaH (60% in mineral oil, 21 mg, 0.53 mmol) in THF (1.0 mL) at room temperature and stirred for 2 h. 6-Chloropyrimidin-4-amine (41 mg, 0.32 mmol) in THF (2.0 mL) was then added, and the reaction was heated to 65° C. and stirred for 18 h. The reaction was cooled to room temperature, quenched with water (10 mL), and extracted with DCM (3×15 mL). The combined organic layers were combined, dried (MgSO₄), and concentrated in vacuo to give the title compound (0.065 g, 87%) which was carried forward to the next step without further purification. ESI-MS (M+H)+: 282.1, ¹H NMR (400 MHz, CDCl₃) δ 8.31 (s, 1H), 7.86 (s, 1H), 7.55 (s, 1H), 7.48 (d, J=9.0 Hz, 1H), 6.96-6.92 (m, 1H), 5.86 (s, 1H), 5.52 (s, 2H), 4.68 (s, 2H), 1.92-1.85 (m, 1H), 0.99-0.93 (m, 2H), 0.70-0.64 (m, 2H).

Synthesis of rac-(1S*,2S*)-2-(3-chlorophenyl)-N-(6-((6-cyclopropylimidazo[1,2-a]pyridin-2-yl)methoxy)pyrimidin-4-yl)cyclopropane-1-carboxamide (I-104). Oxalyl chloride (60 μL, 0.69 mmol) was added to a stirred solution of rac-(1S*,2S*)-2-(3-chlorophenyl)cyclopropane-1-carboxylic acid (45 mg, 0.23 mmol) and DMF (4.5 μL, 0.058 mmol) in DCM (1.0 mL) and stirred at room temperature for 20 min. The reaction was concentrated in vacuo. The residue was dissolved in DCM (2.0 mL) and added to a stirred solution of 6-((6-cyclopropylimidazo[1,2-a]pyridin-2-yl)methoxy)pyrimidin-4-amine (65 mg, 0.23 mmol) in DCM (1.0 mL) at 0° C. Pyridine (0.93 mL, 12 mmol) was then added and the reaction was stirred at 0° C. for 1 h. The reaction was quenched with water (20 mL) and extracted with DCM (3×15 mL). The organic layers were combined, dried over MgSO₄, and concentrated in vacuo. The residue was purified by reverse phase preparative HPLC to give the title compound (39 mg, 37%). ESI-MS (M+H)+: 460.2, ¹H NMR (400 MHz, DMSO) δ 11.17 (s, 1H), 8.61 (d, J=0.9 Hz, 1H), 8.39 (s, 1H), 7.91 (s, 1H), 7.52 (d, J=0.9 Hz, 1H), 7.47 (d, J=9.6 Hz, 1H), 7.41-7.30 (m, 3H), 7.24-7.20 (m, 1H), 7.06 (dd, J=1.7, 9.4 Hz, 1H), 5.53 (s, 2H), 2.53-2.49 (m, 1H), 2.49-2.43 (m, 1H), 2.03-1.95 (m, 1H), 1.61-1.49 (m, 2H), 1.01-0.95 (m, 2H), 0.75-0.72 (m, 2H).

### Synthesis of 6-chloro-N-((6-cyclopropylimidazo[1,2-a]pyridin-2-yl)methyl)-2-(pyrrolidin-1-yl)pyrimidin-4-amine

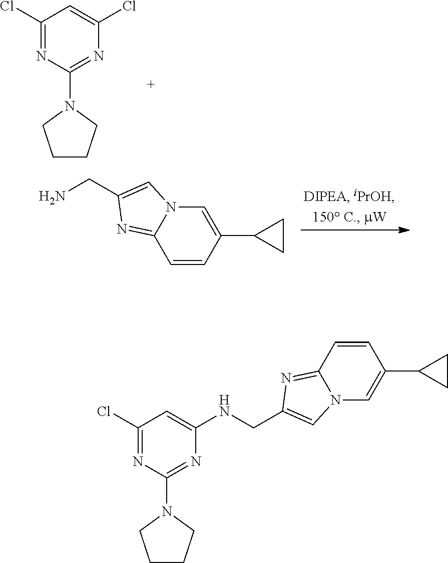

4,6-Dichloro-2-(pyrrolidin-1-yl)pyrimidine (50 mg, 0.23 mmol), (6-cyclopropylimidazo[1,2-a]pyridin-2-yl)methanamine (47 mg, 0.25 mmol) and DIPEA (0.12 mL, 0.69 mmol) in iPrOH (2.0 mL) were heated to 150° C. in a microwave for 2.5 h. The mixture was cooled to room temperature, quenched with water (10 mL), and extracted into EtOAc (2×10 mL). The combined organics were passed through a hydrophobic frit and concentrated in vacuo. The residue was purified by column chromatography on silica gel, eluting with a gradient of 5-100% EtOAc in cyclohexane to give the title compound (40 mg, 47%). ESI-MS (M+H): 369, ¹H NMR (400 MHz, CDCl₃) δ 7.85-7.84 (m, 1H), 7.45-7.40 (m, 2H), 6.93 (dd, J=1.7, 9.2 Hz, 1H), 5.73 (s, 1H), 3.57-3.52 (m, 4H), 1.95-1.85 (m, 5H), 0.99-0.93 (m, 2H), 0.69-0.65 (m, 2H), −0.02 (s, 1H), −0.15 (s, 1H).

Using a similar procedure to that used for 6-chloro-N-((6-cyclopropylimidazo[1,2-a]pyridin-2-yl)methyl)-2-(pyrrolidin-1-yl)pyrimidin-4-amine, the compounds in Table 13 were made from (6-cyclopropylimidazo[1,2-a]pyridin-2-yl)methanamine and an appropriate coupling partner.

TABLE 13

| | Coupling Partner/ |
|---|---|
| Compound | Analytical Data |
| 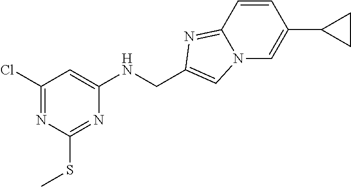 [SMILES: CSc1nc(Cl)cc(NCc2cn3cc(C4CC4)ccc3n2)n1] | 2-(methylthio)-4,6- dichloropyrimidine ESI-MS (M + H): 346 |
| 6-chloro-N-((6-cyclopropylimidazo[1,2-a]pyridin-2-yl)methyl)-2- | |
| (methylthio)pyrimidin-4-amine | |
| 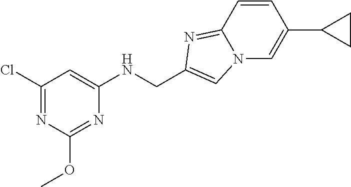 [SMILES: COc1nc(Cl)cc(NCc2cn3cc(C4CC4)ccc3n2)n1] | 2-(methoxy)-4,6- dichloropyrimidine ESI-MS (M + H): 330 |
| 6-chloro-N-((6-cyclopropylimidazo[1,2-a]pyridin-2-yl)methyl)-2- | |
| methoxypyrimidin-4-amine | |
| 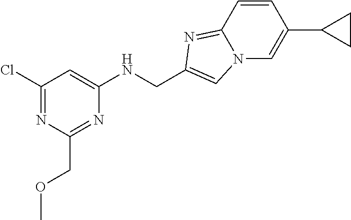 [SMILES: COCc1nc(Cl)cc(NCc2cn3cc(C4CC4)ccc3n2)n1] | 2-(methoxymethyl)-4,6- dichloropyrimidine ESI-MS (M + H): 344 |
| 6-chloro-N-((6-cyclopropylimidazo[1,2-a]pyridin-2-yl)methyl)-2- | |
| (methoxymethyl)pyrimidin-4-amine | |

### Example 105

### Synthesis of rac-(1S*,2S*)-2-(3-chlorophenyl)-N-(6-(((6-cyclopropylimidazo[1,2-a]pyridin-2-yl)methyl)amino)-2-(pyrrolidin-1-yl)pyrimidin-4-yl)cyclopropane-1-carboxamide (I-105)

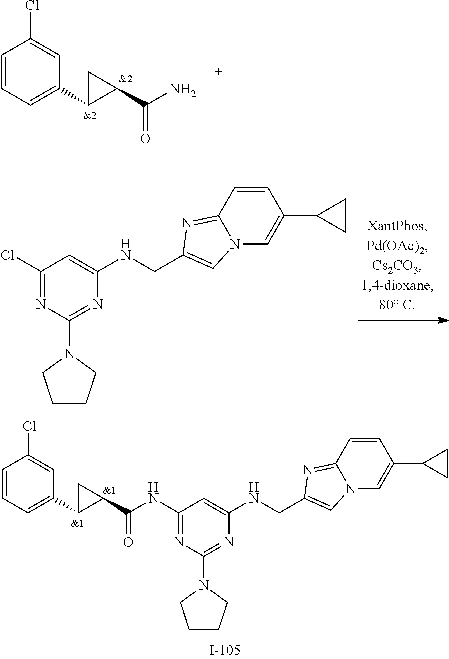

rac-(1S*,2S*)-2-(3-Chlorophenyl)cyclopropane-1-carboxamide (21 mg, 0.11 mmol—prepared in a similar manner to (1S,2S)-2-(3-chlorophenyl)cyclopropane-1-carboxamide), Pd(OAc)₂ (4.9 mg, 0.02 mmol), XantPhos (25 mg, 0.04 mmol), and Cs₂CO₃ (53 mg, 0.16 mmol) were placed under N₂ atmosphere. A solution of 6-chloro-N-((6-cyclopropylimidazo[1,2-a]pyridin-2-yl)methyl)-2-(pyrrolidin-1-yl)pyrimidin-4-amine (40 mg, 0.11 mmol) in 1,4-dioxane (2.5 mL) was added, and the mixture was degassed for 15 min. The reaction mixture was stirred at 80° C. for 2 h. The reaction mixture was allowed to cool to room temperature, filtered through Celite®, washed with MeOH, and concentrated in vacuo. The residue was purified by column chromatography on silica gel, eluting with a gradient of 5-100% EtOAc in cyclohexane. The residue was further purified by preparative HPLC to give the title compound (6.5 mg, 11%) as a formic acid salt. ESI-MS (M+H): 528.3, ¹H NMR (400 MHz, DMSO) δ 9.96 (s, 1H), 8.23 (s, 1H), 7.52 (s, 1H), 7.31-7.20 (m, 3H), 7.17 (d, J=9.1 Hz, 2H), 7.07 (d, J=7.6 Hz, 1H), 6.87 (d, J=9.3 Hz, 1H), 6.53 (s, 1H), 4.46 (s, 2H), 3.37-3.33 (m, 4H), 2.31-2.24 (m, 1H), 1.85-1.80 (m, 1H), 1.77 (s, 4H), 1.40-1.34 (m, 1H), 1.27-1.20 (m, 1H), 0.87-0.80 (m, 2H), 0.58 (q, J=5.2 Hz, 2H) 1 H missing under DMSO peak.

Using a similar procedure to that used for rac-(1S*,2S*)-2-(3-chlorophenyl)-N-(6-(((6-cyclopropylimidazo[1,2-a]pyridin-2-yl)methyl)amino)-2-(pyrrolidin-1-yl)pyrimidin-4-yl)cyclopropane-1-carboxamide, the compounds in Table 14 were prepared from rac-(1S*,2S*)-2-(3-Chlorophenyl)cyclopropane-1-carboxamide and an appropriate coupling partner.

TABLE 14

| | | Coupling Partner/ |
|---|---|---|
| Example | Compound | Analytical Data |
| Example 106 | 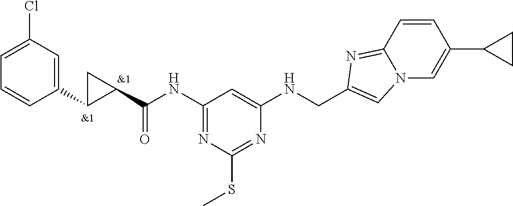 [SMILES: CSc1nc(NCc2cn3cc(C4CC4)ccc3n2)cc(NC(=O)[C@@H]2C[C@H]2c2cccc(Cl)c2)n1] rac-(1S*,2S*)-2-(3-chlorophenyl)-N-(6- (((6-cyclopropylimidazo[1,2-a]pyridin-2- yl)methyl)amino)-2- (methylthio)pyrimidin-4- yl)cyclopropane-1-carboxamide (I-106) | 6-chloro-N-((6- cyclopropylimidazo[1,2-a]pyridin-2- yl)methyl)-2-(methylthio)pyrimidin-4- amine ESI-MS (M + H): 505.4, ¹H NMR (400 MHz, DMSO) δ 10.62 (s, 1H), 8.37 (s, 1H), 8.01-8.00 (m, 1H), 7.67 (s, 1H), 7.43 (d, J = 9.6 Hz, 1H), 7.36 (d, J = 6.8 Hz, 1H), 7.33-7.28 (m, 2H), 7.20 (d, J = 7.3 Hz, 1H), 7.06-7.00 (m, 2H), 4.66-4.62 (s, 2H), 2.48-2.42 (m, 4H), 1.97-1.94 (m, 1H), 1.52-1.51 (m, 1H), 1.45-1.41 (m, 1H), 1.00-0.94 (m, 2H), 0.72-0.71 (m, 2H). One cyclopropyl C—H not observed - possibly under DMSO peak. |
| Example 107 | 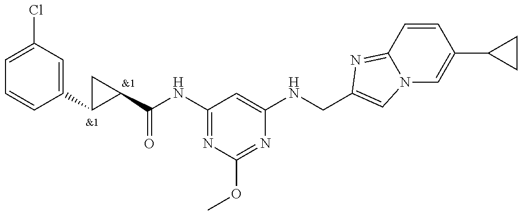 [SMILES: COc1nc(NCc2cn3cc(C4CC4)ccc3n2)cc(NC(=O)[C@@H]2C[C@H]2c2cccc(Cl)c2)n1] rac-(1S*,2S*)-2-(3-chlorophenyl)-N-(6- (((6-cyclopropylimidazo[1,2-a]pyridin-2- yl)methyl)amino)-2-methoxypyrimidin- 4-yl)cyclopropane-1-carboxamide (I- 107) | 6-chloro-N-((6- cyclopropylimidazo[1,2-a]pyridin-2- yl)methyl)-2-methoxypyrimidin-4- amine ESI-MS (M + H): 489.3, ¹H NMR (400 MHz, DMSO) δ 10.53 (s, 1H), 8.35 (s, 1H), 7.67 (s, 1H), 7.43 (d, J = 9.0 Hz, 1H), 7.36 (d, J = 7.8 Hz, 1H), 7.31 (s, 1H), 7.30 (s, 1H), 7.20 (d, J = 7.8 Hz, 1H), 7.03 (s, 1H), 7.01 (s, 1H), 6.60 (s, 1H), 4.61 (br s, 2H), 3.82 (s, 3H), 2.44 (t, J = 7.0 Hz, 2H), 2.00-1.92 (m, 1H), 1.54-1.50 (m, 1H), 1.46-1.41 (m, 1H), 0.96 (q, J = 6.2 Hz, 2H), 0.75-0.69 (m, 2H). |
| Example 108 | 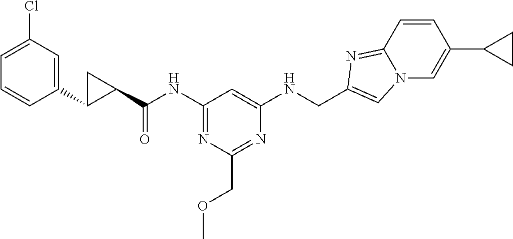 [SMILES: COCc1nc(NCc2cn3cc(C4CC4)ccc3n2)cc(NC(=O)[C@@H]2C[C@H]2c2cccc(Cl)c2)n1] | 6-chloro-N-((6- cyclopropylimidazo[1,2-a]pyridin-2- yl)methyl)-2- (methoxymethyl)pyrimidin-4-amine ESI-MS (M + H): 503.3, ¹H NMR (400 MHz, DMSO) δ 10.70 (s, 1H), 8.30 (s, 1H), 7.82 (s, 1H), 7.62 (s, 1H), 7.37 (d, J = 9.3 Hz, 1H), 7.31 (d, J = 8.0 Hz, 1H), 7.26 (s, 1H), 7.25 (s, 1H), 7.19-7.11 (m, 2H), 6.96 (d, J = 9.1 Hz, 1H), 4.57 (br s, 2H), 4.23 (s, 2H), 2.39 (t, J = 7.1 Hz, 2H), 1.95-1.86 (m, 1H), 1.48- 1.43 (m, 1H), 1.40-1.33 (m, 1H) 0.94- 0.87 (m, 2H), 0.69-0.63 (m, 2H). *3.37 (s, 3H) vsible after D₂O shake. |
| | rac-(1S*,2S*)-2-(3-chlorophenyl)-N-(6- | |
| | (((6-cyclopropylimidazo[1,2-a]pyridin-2- | |
| | yl)methyl)amino)-2- | |
| | (methoxymethyl)pyrimidin-4- | |
| | yl)cyclopropane-1-carboxamide (I-108) | |

### Examples 109-110

### Synthesis of rac-(1S*,2S*)-2-(3-chlorophenyl)-N-(6-(((6-cyclopropylimidazo[1,2-a]pyridin-2-yl)methyl)amino)-2-(methylsulfinyl)pyrimidin-4-yl)cyclopropane-1-carboxamide (I-109) and rac-(methylsulfonyl)pyrimidin-4-yl)cyclopropane-1-carboxamide (I-110)

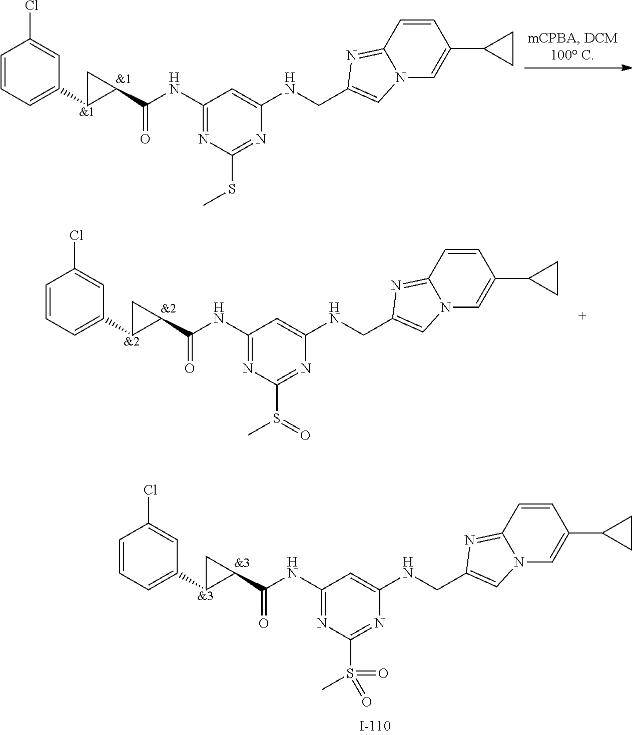 [SMILES: CSc1nc(NCc2cn3cc(C4CC4)ccc3n2)cc(NC(=O)[C@@H]2C[C@H]2c2cccc(Cl)c2)n1 ; CS(=O)c1nc(NCc2cn3cc(C4CC4)ccc3n2)cc(NC(=O)[C@@H]2C[C@H]2c2cccc(Cl)c2)n1 ; CS(=O)(=O)c1nc(NCc2cn3cc(C4CC4)ccc3n2)cc(NC(=O)[C@@H]2C[C@H]2c2cccc(Cl)c2)n1]

mCPBA (68 mg, 0.40 mmol) was added to a solution of rac-(1S*,2S*)-2-(3-chlorophenyl)-N-(6-(((6-cyclopropylimidazo[1,2-a]pyridin-2-yl)methyl)amino)-2-(methylthio)pyrimidin-4-yl)cyclopropane-1-carboxamide (100 mg, 0.20 mmol) in DCM (3.0 mL) at 0° C. The reaction mixture was stirred for 4 h at 0° C. The reaction mixture was allowed to warm to room temperature, quenched with water (10 mL), and extracted with DCM (3×10 mL). The combined organics were passed through a hydrophobic frit and concentrated in vacuo. The residue was purified by preparative HPLC to give rac-(1S*,2S*)-(3-chlorophenyl)-N-(6-(((6-cyclopropylimidazo[1,2-a]pyridin-2-yl)methyl)amino)-2-(methylsulfinyl)pyrimidin-4-yl)cyclopropane-1-carboxamide (11.2 mg, 11%) and rac-(1S*,2S*)-2-(3-chlorophenyl)-N-(6-(((6-cyclopropylimidazo[1,2-a]pyridin-2-yl)methyl)amino)-2-(methylsulfonyl)pyrimidin-4-yl)cyclopropane-1-carboxamide (4.2 mg, 4%).

rac-(1S*,2S*)-2-(3-chlorophenyl)-N-(6-(((6-cyclopropylimidazo[1,2-a]pyridin-2-yl)methyl)amino)-2-(methylsulfinyl)pyrimidin-4-yl)cyclopropane-1-carboxamide:

ESI-MS (M+H): 521.3, ¹H NMR (400 MHz, DMSO) δ 11.04 (s, 1H), 8.38 (s, 1H), 8.30 (s, 1H), 7.66 (s, 1H), 7.37 (d, J=9.9 Hz, 1H), 7.35-7.30 (m, 2H), 7.28-7.23 (m, 2H), 7.15 (d, J=7.6 Hz, 1H), 6.97 (d, J=9.1 Hz, 1H), 4.62 (br s, 2H), 2.76 (s, 3H), 2.44-2.40 (m, 2H), 1.93-1.88 (m, 1H), 1.51-1.47 (m, 1H), 1.43-1.39 (m, 1H), 0.94-0.88 (m, 2H), 0.69-0.64 (m, 2H).

rac-(1S*,2S*)-2-(3-chlorophenyl)-N-(6-(((6-cyclopropylimidazo[1,2-a]pyridin-2-yl)methyl)amino)-2-(methylsulfonyl)pyrimidin-4-yl)cyclopropane-1-carboxamide:

ESI-MS (M+H): 537.3, ¹H NMR (400 MHz, DMSO) δ 11.14 (s, 1H), 8.64 (s, 1H), 8.34 (s, 1H), 7.72 (s, 1H), 7.46 (s, 1H), 7.42 (d, J=8.8 Hz, 1H), 7.34 (d, J=7.1 Hz, 1H), 7.34-7.29 (m, 2H), 7.19 (d, J=6.6 Hz, 1H), 7.00 (d, J=9.1 Hz, 1H), 4.78 (br s, 2H), 3.29 (s, 3H), 2.50-2.40 (m, 2H), 1.99-1.92 (m, 1H), 1.58-1.50 (m, 1H), 1.50-1.41 (m, 1H), 0.99-0.90 (m, 2H), 0.74-0.67 (m, 2H).

### Example 111

### Synthesis of rac-ethyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate (I-111)

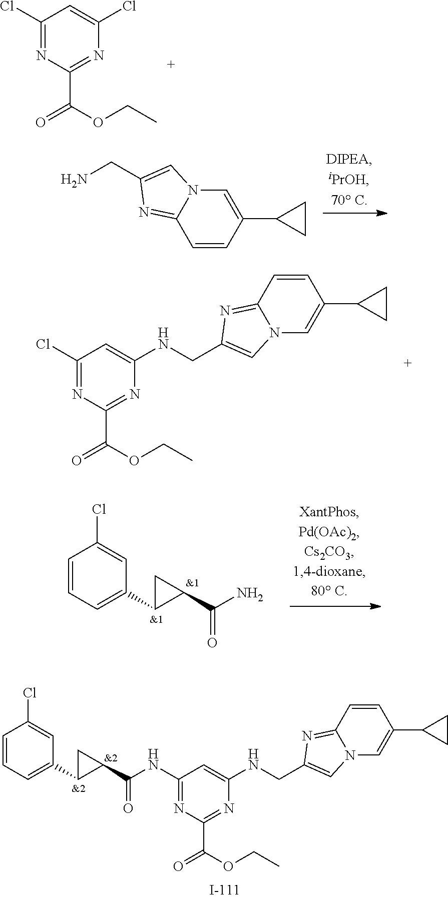

Synthesis of ethyl 4-chloro-6-(((6-cyclopropylimidazo[1,2-a]pyridin-2-yl)methyl)amino)pyrimidine-2-carboxylate. A solution of ethyl 4,6-dichloropyrimidine-2-carboxylate (200 mg, 0.91 mmol), (6-cyclopropylimidazo[1,2-a]pyridin-2-yl)methanamine (140 mg, 0.75 mmol) and DIPEA (0.33 mL, 1.9 mmol) in iPrOH (10 mL) was heated to 70° C. for 1.5 h. The mixture was cooled to room temperature and concentrated in vacuo. The residue was dissolved in 5% MeOH in DCM (60 mL) and washed with water (15 mL). The organics were passed through a hydrophobic frit and concentrated in vacuo. The residue was triturated with Et₂O (10 mL) to give the title compound (190 mg, 68%) as a beige solid. ESI-MS (M+H): 372.1, ¹H NMR (400 MHz, CDCl₃) δ 7.86 (s, 1H), 7.46-7.42 (m, 2H), 6.97 (dd, J=1.7, 9.3 Hz, 1H), 6.54 (s, 1H), 6.33-6.33 (m, 1H), 4.70-4.67 (m, 2H), 4.48 (q, J=7.2 Hz, 2H), 1.93-1.85 (m, 1H), 1.44 (dd, J=7.1, 7.1 Hz, 3H), 1.00-0.94 (m, 2H), 0.70-0.65 (m, 2H).

Synthesis of rac-ethyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate (I-111). rac-(1S*,2S*)-2-(3-chlorophenyl)cyclopropane-1-carboxamide (70 mg, 0.19 mmol), ethyl 4-chloro-6-(((6-cyclopropylimidazo[1,2-a]pyridin-2-yl)methyl)amino)pyrimidine-2-carboxylate (37 mg, 0.19 mmol), Pd(OAc)₂ (8.5 mg, 0.04 mmol), XantPhos (44 mg, 0.08 mmol), and Cs₂CO₃ (92 mg, 0.282 mmol) were placed under N₂ atmosphere, 1,4-dioxane (5.0 mL) was added, and the mixture was degassed for 5 min. The reaction mixture was stirred at 90° C. for 2 h. The reaction mixture was allowed to cool to room temperature, diluted with 5% MeOH in DCM (50 mL), filtered through Celite®, and concentrated in vacuo. The residue was purified by column chromatography on silica gel, eluting with a gradient of 0-10% EtOH in DCM. The residue was triturated with iPr₂O to give the title compound (40 mg, 40%). ESI-MS (M+H): 531.5, ¹H NMR (400 MHz, DMSO) 11.00 (s, 1H), 8.31 (s, 1H), 8.22 (s, 1H), 7.66 (s, 1H), 7.42-7.26 (m, 5H), 7.15 (d, J=7.5 Hz, 1H), 6.98 (dd, J=1.7, 9.3 Hz, 1H), 4.64 (s, 2H), 4.30 (q, J=7.1 Hz, 2H), 2.47-2.39 (m, 2H), 1.97-1.89 (m, 1H), 1.49-1.37 (m, 2H), 1.30 (t, J=7.1 Hz, 3H), 0.95-0.89 (m, 2H), 0.70-0.65 (m, 2H).

### Example 112

### Synthesis of rac-4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylic acid (I-112)

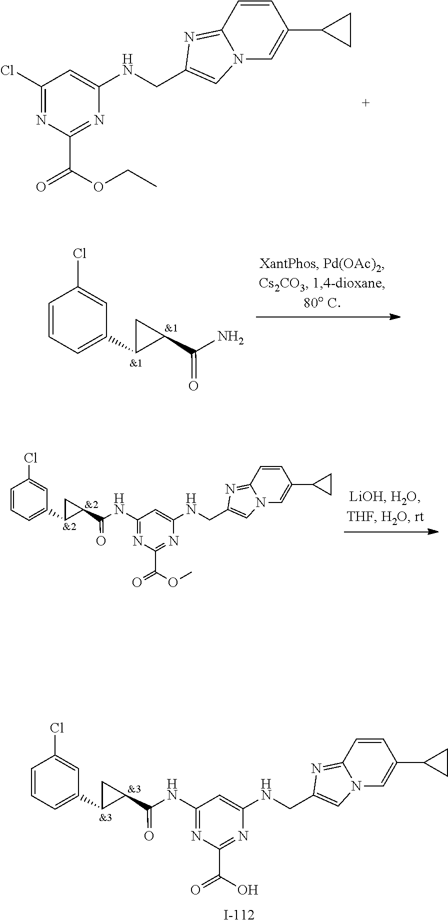 [SMILES: CCOC(=O)c1nc(Cl)cc(NCc2cn3cc(C4CC4)ccc3n2)n1 ; NC(=O)[C@@H]1C[C@H]1c1cccc(Cl)c1 ; COC(=O)c1nc(NCc2cn3cc(C4CC4)ccc3n2)cc(NC(=O)[C@@H]2C[C@H]2c2cccc(Cl)c2)n1 ; O=C(O)c1nc(NCc2cn3cc(C4CC4)ccc3n2)cc(NC(=O)[C@@H]2C[C@H]2c2cccc(Cl)c2)n1]

Synthesis of rac-methyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate. rac-(1S*,2S*)-2-(3-chlorophenyl)cyclopropane-1-carboxamide (70 mg, 0.19 mmol), ethyl 4-chloro-6-(((6-cyclopropylimidazo[1,2-a]pyridin-2-yl)methyl)amino)pyrimidine-2-carboxylate (37 mg, 0.19 mmol), Pd(OAc)₂ (8.5 mg, 0.04 mmol), XantPhos (44 mg, 0.08 mmol), and Cs₂CO₃ (92 mg, 0.282 mmol) were placed under N₂ atmosphere, 1,4-dioxane (5.0 mL) was added, and the mixture was degassed for 5 min. The reaction mixture was stirred at 80° C. for 1.5 h. The reaction mixture was allowed to cool to room temperature, diluted with 10% MeOH in DCM (50 mL), filtered through Celite®, and concentrated in vacuo. Transesterification to the methyl ester was observed after workup. The residue was purified by column chromatography on silica gel, eluting with a gradient of 0-10% MeOH in DCM to give the title compound (40 mg, 40%) as a colourless oil. ESI-MS (M+H): 531.2 ¹H NMR (400 MHz, CDCl₃) δ 8.39-8.39 (m, 1H), 7.85 (s, 1H), 7.48 (s, 1H), 7.44 (d, J=9.3 Hz, 2H), 7.20 (dd, J=7.5, 7.5 Hz, 2H), 7.08 (s, 1H), 7.00-6.97 (m, 1H), 6.93 (dd, J=1.7, 9.3 Hz, 1H), 6.11-6.11 (m, 1H), 4.69-4.69 (m, 2H), 3.99 (s, 3H), 2.60-2.54 (m, 1H), 1.92-1.84 (m, 1H), 1.76-1.67 (m, 2H), 1.45-1.34 (m, 1H), 0.99-0.93 (m, 2H), 0.69-0.64 (m, 2H).

Synthesis of rac-4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylic acid (I-112). A solution of rac-methyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate (40 mg, 0.08 mmol) and LiOH·H₂O (3.6 mg, 0.09 mmol) in THF (2.0 mL) and water (0.4 mL) was stirred at room temperature for 4 h. A solution of HCl (4 M in 1,4-dioxane, 20 μL) was added to the reaction mixture, which was stirred for 5 min, then concentrated in vacuo. The residue was purified by preparative HPLC to give the title compound (12 mg, 31%) as a formic acid salt. ESI-MS (M+H): 503.3, ¹H NMR (400 MHz, DMSO) δ 10.96 (s, 1H), 8.32 (s, 1H), 8.11 (s, 1H), 7.66 (s, 1H), 7.39 (d, J=9.4 Hz, 1H), 7.35-7.30 (m, 2H), 7.28-7.26 (m, 2H), 7.15 (d, J=7.7 Hz, 1H), 6.98 (dd, J=1.8, 9.3 Hz, 1H), 4.65-4.64 (m, 2H), 2.44-2.38 (m, 2H), 1.96-1.89 (m, 1H), 1.52-1.38 (m, 2H), 0.95-0.89 (m, 2H), 0.70-0.66 (m, 2H)

### Examples 113-114

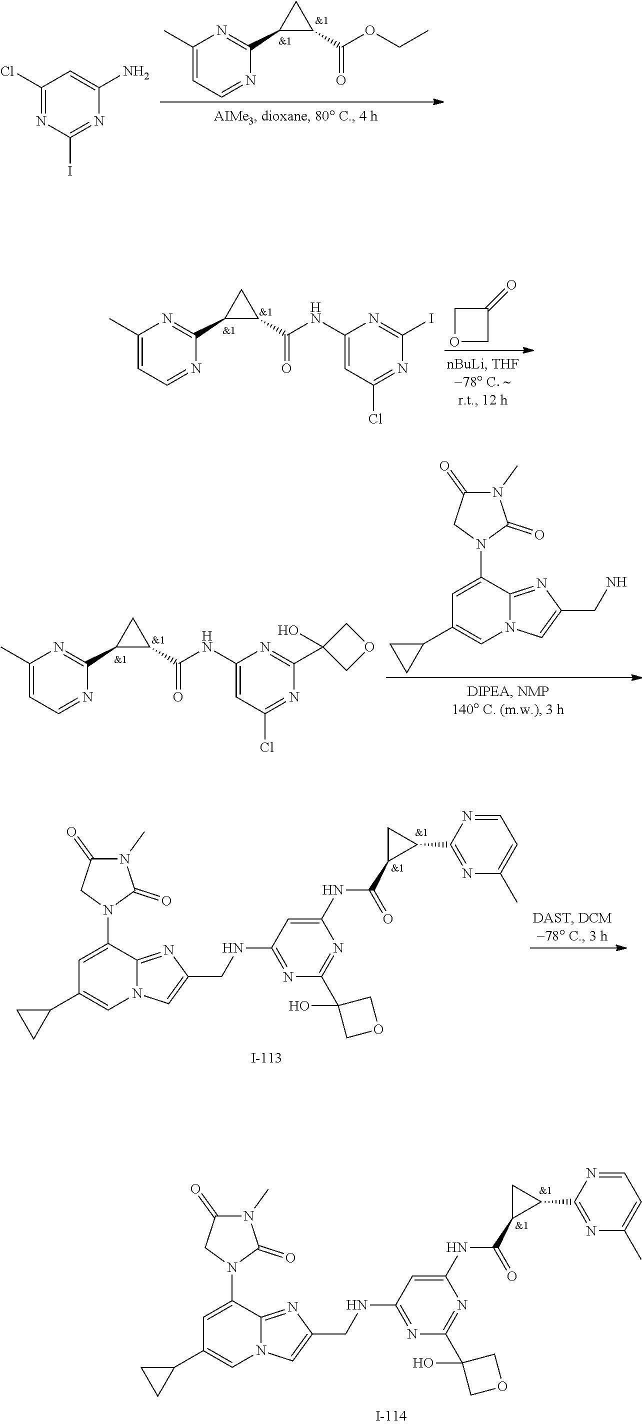

### Synthesis of rac-(1S*,2S*)—N-(6-chloro-2-iodopyrimidin-4-yl)-2-(4-methylpyrimidin-2-yl)cyclopropane-1-carboxamide

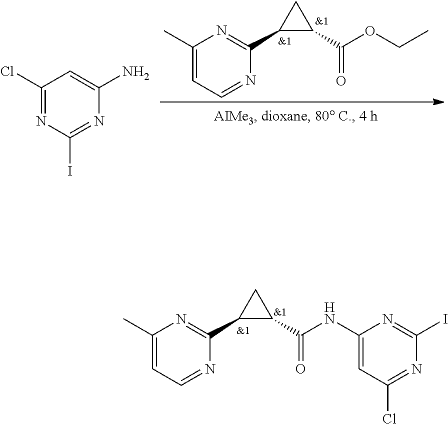

To a mixture of 6-chloro-2-iodopyrimidin-4-amine (3 g, 11.74 mmol) in dioxane (50 mL) was added AlMe3 (4.35 mL, 13.05 mmol) at 0° C. under N₂. After the reaction mixture was stirred at 30° C. for 2 h, a solution of rac-ethyl (1S*,2S*)-2-(4-methylpyrimidin-2-yl)cyclopropane-1-carboxylate (900 mg, 4.36 mmol) in dioxane (5 mL) was added. The resulting mixture was stirred at 90° C. for 6 h, then quenched with water (50 mL) and extracted with EtOAc (3×50 mL). The combined organic layers were washed with brine (100 mL), dried over Na₂SO₄, and concentrated to give the crude, which was purified by column chromatography (eluent: PE/EtOAc=5:1) to give rac-(1S*,2S*)—N-(6-chloro-2-iodopyrimidin-4-yl)-2-(4-methylpyrimidin-2-yl)cyclopropane-1-carboxamide as a white solid (750 mg, 41.3%). ESI-MS [M+H]+: 416

### Synthesis of rac-(1S*,2S*)—N-(6-chloro-2-(3-hydroxyoxetan-3-yl)pyrimidin-4-yl)-2-(4-methylpyrimidin-2-yl)cyclopropane-1-carboxamide

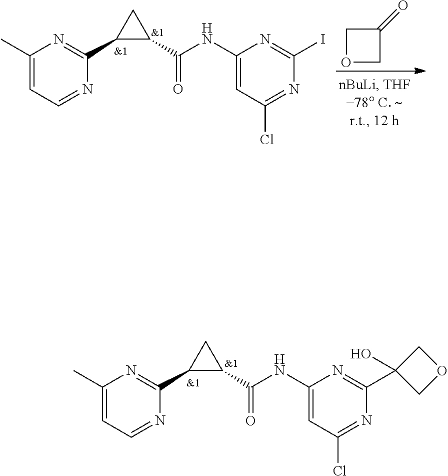

To a solution of rac-(1S*,2S*)—N-(6-chloro-2-iodopyrimidin-4-yl)-2-(4-methylpyrimidin-2-yl)cyclopropane-1-carboxamide (50 mg, 0.12 mmol) in THF (10 mL) was added nBuLi (0.23 mL, 0.58 mmol) at −78° C. for 1 h. Oxetan-3-one (173 mg, 2.40 mmol) was then added and the mixture was stirred for another 3 h. The reaction mixture was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC (eluent: DCM/MeOH=10/1) to give rac-(1S*,2S*)—N-(6-chloro-2-(3-hydroxyoxetan-3-yl)pyrimidin-4-yl)-2-(4-methylpyrimidin-2-yl)cyclopropane-1-carboxamide as a white solid (10 mg, 23%) ESI-MS [M+H]+: 362

### Synthesis of rac-(1S*,2S*)—N-(6-chloro-2-(3-hydroxyoxetan-3-yl)pyrimidin-4-yl)-2-(4-methylpyrimidin-2-yl)cyclopropane-1-carboxamide (I-113)

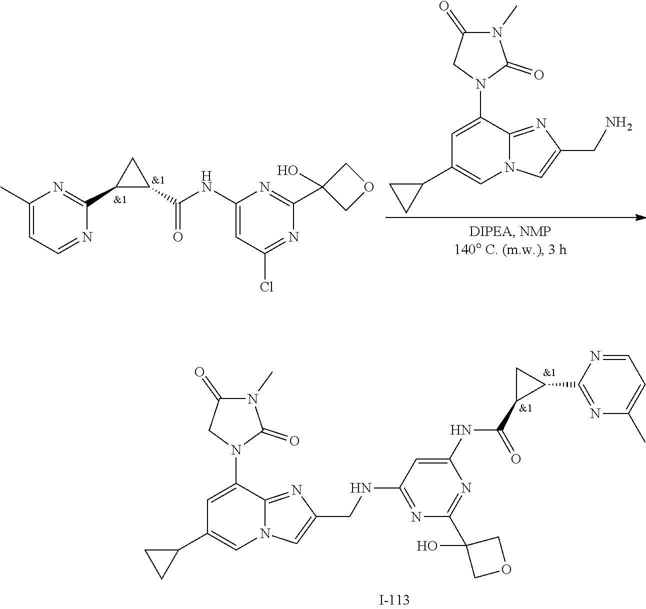

A mixture of rac-(1S*,2S*)—N-(6-chloro-2-(3-hydroxyoxetan-3-yl)pyrimidin-4-yl)-2-(4-methylpyrimidin-2-yl)cyclopropane-1-carboxamide (20 mg, 0.055 mmol), 1-(2-(aminomethyl)-6-cyclopropylimidazo[1,2-a]pyridin-8-yl)-3-methylimidazolidine-2,4-dione (24 mg, 0.080 mmol), and DIPEA (34.9 mg, 0.27 mmol) in i-PrOH (5 mL) was stirred in a sealed tube. After degassing with N₂ for 1 min, the reaction was irradiated in microwave at 140° C. for 3 h. The reaction mixture was cooled to room temperature and concentrated in vacuo to give the crude, which was purified by preparative TLC (eluent: DCM/MeOH=10/1) to give rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-hydroxyoxetan-3-yl)pyrimidin-4-yl)-2-(4-methylpyrimidin-2-yl)cyclopropane-1-carboxamide as a white solid (20 mg, 58.2%). ESI-MS [M+H]+: 625.2, ¹H NMR (400 MHz, CD3OD) δ 8.45 (d, J=5.2 Hz, 1H), 8.14 (s, 1H), 7.73 (s, 1H), 7.36-7.32 (m, 1H), 7.26 (s, 1H), 7.16 (d, J=5.2 Hz, 1H), 5.00 (d, J=6.2 Hz, 2H), 4.71 (d, J=6.2 Hz, 3H), 4.66-4.58 (m, 3H), 3.08 (s, 3H), 2.71-2.67 (m, 1H), 2.53-2.49 (m, 1H), 2.46 (s, 3H), 1.95-1.92 (m, 1H), 1.68-1.63 (m, 2H), 0.99-0.94 (m, 2H), 0.74-0.71 (m, 2H).

### Synthesis of rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-fluorooxetan-3-yl)pyrimidin-4-yl)-2-(4-methylpyrimidin-2-yl)cyclopropane-1-carboxamide (I-114)

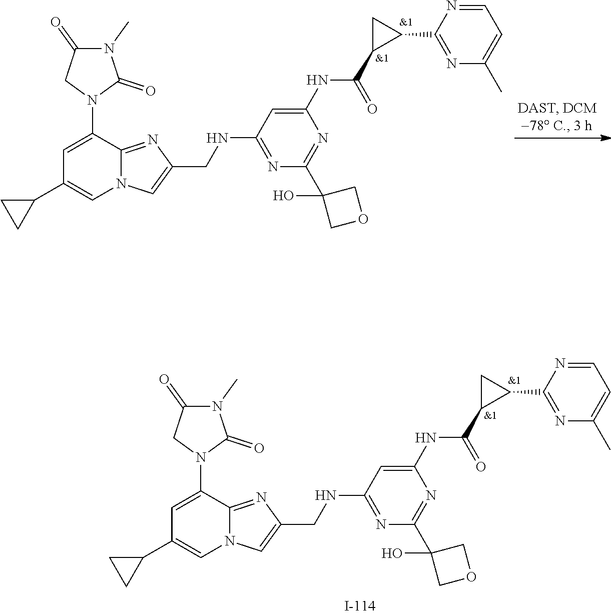 [SMILES: Cc1ccnc([C@H]2C[C@@H]2C(=O)Nc2cc(NCc3cn4cc(C5CC5)cc(N5CC(=O)N(C)C5=O)c4n3)nc(C3(O)COC3)n2)n1 ; Cc1ccnc([C@H]2C[C@@H]2C(=O)Nc2cc(NCc3cn4cc(C5CC5)cc(N5CC(=O)N(C)C5=O)c4n3)nc(C3(O)COC3)n2)n1]

To a mixture of rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-hydroxyoxetan-3-yl)pyrimidin-4-yl)-2-(4-methylpyrimidin-2-yl)cyclopropane-1-carboxamide (32 mg, 0.051 mmol) in DCM (10 mL) was added DAST (80 mg, 0.496 mmol) at −78° C. After stirring at −78° C. for 3 h, the resulting mixture was quenched with water (10 mL) and extracted with EtOAc (3×10 mL). The combined organic layers were washed with brine (20 mL), dried over anhydrous Na₂SO₄, and concentrated to give the crude product, which was purified by preparative TLC (eluent: DCM/MeOH=10/1) to give rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-fluorooxetan-3-yl)pyrimidin-4-yl)-2-(4-methylpyrimidin-2-yl)cyclopropane-1-carboxamide (17 mg, yield 53.2%) as a white solid. ESI-MS [M+H]+: 627.3, ¹H NMR (400 MHz, CD3OD) δ 8.45 (d, J=5.2 Hz, 1H), 8.15 (s, 1H), 7.71 (s, 1H), 7.29 (d, J=14.8 Hz, 2H), 7.15 (d, J=5.2 Hz, 1H), 5.12 (dd, J=7.7, 2.8 Hz, 1H), 5.07 (dd, J=8.4, 2.0 Hz, 1H), 4.93-4.91 (m, 1H), 4.81-4.80 (m, 1H), 4.69-4.62 (m, 3H), 4.53-4.48 (m, 1H), 3.08 (s, 3H), 2.71-2.67 (m, 1H), 2.53-2.49 (m, 1H), 2.46 (s, 3H), 1.68-1.62 (m, 2H), 0.98-0.94 (m, 2H), 0.74-0.70 (m, 2H).

### Synthesis of 2-chloro-N-((6-cyclopropylimidazo[1,2-a]pyridin-2-yl)methyl)pyridin-4-amine

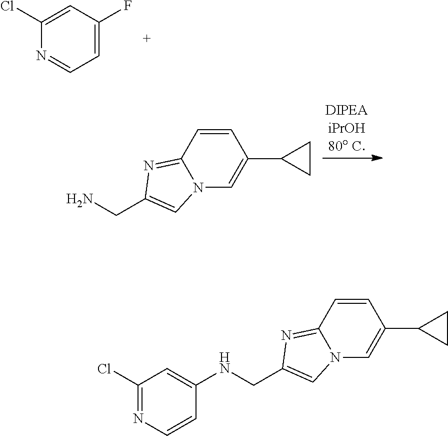 [SMILES: Fc1ccnc(Cl)c1 ; NCc1cn2cc(C3CC3)ccc2n1 ; Clc1cc(NCc2cn3cc(C4CC4)ccc3n2)ccn1]

A mixture of (6-cyclopropylimidazo[1,2-a]pyridin-2-yl)methanamine (0.30 g, 1.6 mmol), 2-chloro-4-fluoropyridine (0.14 mL, 1.6 mmol), and DIPEA (0.56 mL, 3.2 mmol) in iPrOH (16 mL) was stirred at 80° C. for 24 h. Water was added and the mixture was extracted with DCM (3×30 mL). The combined organic layers were dried (MgSO₄), filtered, and concentrated in vacuo. The residue was purified by column chromatography on silica gel, eluting with 0-6% MeOH in DCM to give the title compound (0.046 g, 20%) as a yellow oil. ¹H NMR (400 MHz, CDCl₃) δ 7.95 (d, J=5.8 Hz, 1H), 7.86 (s, 1H), 7.45 (d, J=9.3 Hz, 1H), 7.41 (s, 1H), 6.96 (dd, J=1.8, 9.3 Hz, 1H), 6.54 (d, J=2.0 Hz, 1H), 6.44 (dd, J=2.3, 5.8 Hz, 1H), 5.12 (dd, J=5.4, 5.4 Hz, 1H), 4.47 (d, J=5.3 Hz, 2H), 1.93-1.85 (m, 1H), 0.97 (ddd, J=4.9, 6.3, 8.4 Hz, 2H), 0.70-0.65 (m, 2H).

The compounds in Table 15 were synthesized using a similar procedure to that used for 2-chloro-N-((6-cyclopropylimidazo[1,2-a]pyridin-2-yl)methyl)pyridin-4-amine from 2-chloro-4-fluoropyridine and an appropriate coupling partner.

TABLE 15

| | Coupling Partner/ |
|---|---|
| Compound | Analytical Data |
| 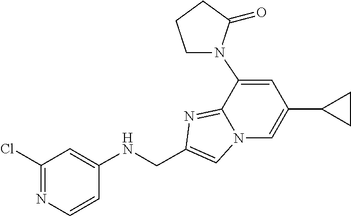 [SMILES: O=C1CCCN1c1cc(C2CC2)cn2cc(CNc3ccnc(Cl)c3)nc12] | 1-(2-(aminomethyl)-6-cyclopropylimidazo[1,2- a]pyridin-8-yl)pyrrolidin-2-one ESI-MS (M + H)+: 382.2, ¹H NMR (400 MHz, CDCl₃) δ 7.95 (d, J = 5.8 Hz, 1H), 7.75 (s, 1H), 7.39 (s, 1H), 7.21 (d, J = 1.6 Hz, 1H), 6.58 (d, J = 2.1 Hz, 1H), 6.44 (dd, J = 2.2, 5.8 Hz, 1H), 5.21 (dd, J = 4.9, 4.9 Hz, 1H), 4.44 (d, J = 5.1 Hz, 2H), 4.25 (dd, J = 7.1, 7.1 Hz, 2H), 2.63 (dd, J = 8.2, 8.2 Hz, 2H), 2.30-2.20 (m, 2H), 1.93-1.85 (m, 1H), 0.99-0.92 (m, 2H), 0.72-0.67 (m, 2H). |
| 1-(2-(((2-chloropyridin-4- | |
| yl)amino)methyl)-6- | |
| cyclopropylimidazo[1,2-a]pyridin-8- | |
| yl)pyrrolidin-2-one | |
| 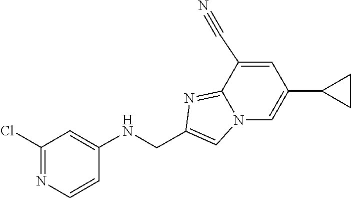 [SMILES: N#Cc1cc(C2CC2)cn2cc(CNc3ccnc(Cl)c3)nc12] | 2-(aminomethyl)-6-cyclopropylimidazo[1,2- a]pyridine-8-carbonitrile ESI-MS (M + H)+: 324 ¹H NMR (400 MHz, CDCl₃) δ 8.07 (s, 1H), 7.96-7.92 (m, 1H), 7.54 (s, 1H), 7.42 (d, J = 1.5 Hz, 1H), 6.51 (d, J = 2.0 Hz, 1H), 6.44 (dd, J = 2.3, 5.8 Hz, 1H), 5.48 (dd, J = 5.4, 5.4 Hz, 1H), 4.54 (d, J = 5.3 Hz, 2H), 1.97-1.87 (m, 1H), 1.08-1.02 (m, 2H), 0.74-0.68 (m, 2H). |
| 2-(((2-chloropyridin-4-yl)amino)methyl)-6- | |
| cyclopropylimidazo[1,2-a]pyridine-8- | |
| carbonitrile | |

### Synthesis of rac-(1S*,2S*)-2-(3-chlorophenyl)-N-(4-(((6-cyclopropylimidazo[1,2-a]pyridin-2-yl)methyl)amino)pyridin-2-yl)cyclopropane-1-carboxamide (I-115)

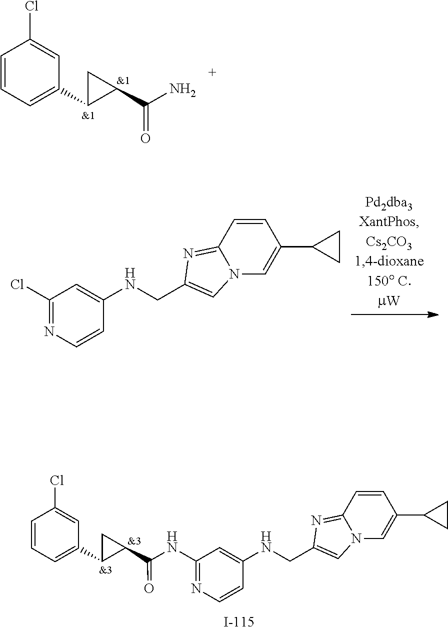 [SMILES: NC(=O)[C@@H]1C[C@H]1c1cccc(Cl)c1 ; Clc1cc(NCc2cn3cc(C4CC4)ccc3n2)ccn1 ; O=C(Nc1cc(NCc2cn3cc(C4CC4)ccc3n2)ccn1)[C@@H]1C[C@H]1c1cccc(Cl)c1]

A mixture of 2-chloro-N-((6-cyclopropylimidazo[1,2-a]pyridin-2-yl)methyl)pyridin-4-amine (0.070 g, 0.23 mmol), rac-(1S*,2S*)-2-(3-chlorophenyl)cyclopropane-1-carboxamide (0.062 g, 0.28 mmol), Pd₂(dba)₃ (0.032 g, 0.035 mmol), XantPhos (0.041 g, 0.070 mmol), and Cs₂CO₃ (0.15 g, 0.47 mmol) in 1,4-dioxane (2.5 mL) and DMF (0.5 mL) was degassed with nitrogen, then stirred at 150° C. in a microwave for 1 h. The mixture was diluted with a mixture of methanol in DCM (1:9) and filtered through Celite®. The mixture was washed with water then concentrated in vacuo. The residue was purified by preparative HPLC to give the title compound (0.010 g, 9%, beige solid) as a mixture of enantiomers. ESI-MS (M+H)+: 458.3, ¹H NMR (400 MHz, DMSO) δ 10.30 (s, 1H), 8.36 (s, 1H), 8.31 (s, 1H), 7.75 (d, J=5.8 Hz, 1H), 7.63 (s, 1H), 7.44-7.22 (m, 5H), 7.16-7.08 (m, 2H), 6.97 (dd, J=1.6, 9.2 Hz, 1H), 6.32 (dd, J=1.9, 5.8 Hz, 1H), 4.35 (d, J=5.8 Hz, 2H), 2.39-2.31 (m, 2H), 1.94-1.87 (m, 1H), 1.47-1.41 (m, 1H), 1.37-1.31 (m, 1H), 0.93-0.87 (m, 2H), 0.69-0.64 (m, 2H).

The mixture was separated using SFC (YMC Cellulose-C 10×250 mm, 5 μm 55/45 MeOH (0.1% DEA)/CO₂, 15 mL/min, 120 bar, 40° C.) to give two enantiomers: (1S,2S)-2-(3-chlorophenyl)-N-(4-(((6-cyclopropylimidazo[1,2-a]pyridin-2-yl)methyl)amino)pyridin-2-yl)cyclopropane-1-carboxamide and (1R,2R)-2-(3-chlorophenyl)-N-(4-(((6-cyclopropylimidazo[1,2-a]pyridin-2-yl)methyl)amino)pyridin-2-yl)cyclopropane-1-carboxamide.

First eluting isomer, (1R,2R)-2-(3-chlorophenyl)-N-(4-(((6-cyclopropylimidazo[1,2-a]pyridin-2-yl)methyl)amino)pyridin-2-yl)cyclopropane-1-carboxamide (I-116):

ESI-MS (M+H)+: 458.2, ¹H NMR (400 MHz, DMSO) δ 10.36 (s, 1H), 8.36 (s, 1H), 7.81 (d, J=5.8 Hz, 1H), 7.68 (s, 1H), 7.51-7.28 (m, 6H), 7.22-7.12 (m, 2H), 7.05-7.00 (m, 1H), 6.38 (dd, J=2.3, 5.8 Hz, 1H), 4.41 (d, J=5.8 Hz, 2H), 2.46-2.37 (m, 2H), 2.01-1.92 (m, 1H), 1.53-1.46 (m, 1H), 1.43-1.36 (m, 1H), 1.00-0.92 (m, 2H), 0.74-0.68 (m, 2H), Enantiomer RT=3.00 min, 99.9% e.e. Formic acid salt

Second eluting isomer, (1S,2S)-2-(3-chlorophenyl)-N-(4-(((6-cyclopropylimidazo[1,2-a]pyridin-2-yl)methyl)amino)pyridin-2-yl)cyclopropane-1-carboxamide (I-117):

ESI-MS (M+H)+: 458.2, ¹H NMR (400 MHz, DMSO) δ 10.35 (s, 1H), 8.35 (s, 1H), 7.80 (d, J=5.6 Hz, 1H), 7.68 (s, 1H), 7.48 (s, 1H), 7.43 (d, J=9.3 Hz, 1H), 7.36 (dd, J=7.6, 7.6 Hz, 1H), 7.31-7.26 (m, 2H), 7.22-7.12 (m, 2H), 7.04-6.99 (m, 1H), 6.39-6.35 (m, 1H), 4.42 (d, J=4.5 Hz, 2H), 2.44-2.40 (m, 2H), 2.01-1.92 (m, 1H), 1.55-1.46 (m, 1H), 1.41-1.34 (m, 1H), 0.99-0.91 (m, 2H), 0.75-0.70 (m, 2H). Enantiomer RT=3.91 min, 99.9% e.e.

Using a similar procedure to that used for rac-(1S*,2S*)-2-(3-chlorophenyl)-N-(4-(((6-cyclopropylimidazo[1,2-a]pyridin-2-yl)methyl)amino)pyridin-2-yl)cyclopropane-1-carboxamide, the compounds in Table 16 were prepared from rac-(1S*,2S*)-2-3-chlorophenyl)cyclopropane-1-carboxamide and the indicated coupling partner, then purified by preparative LCMS.

TABLE 16

| | | Coupling Partner/ |
|---|---|---|
| Example | Compound | Analytical Data |
| Example 118 | 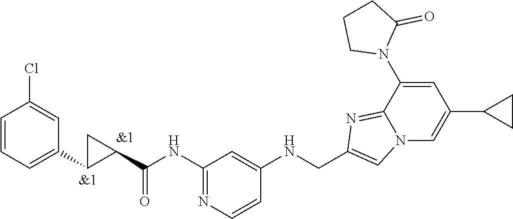 [SMILES: O=C(Nc1cc(NCc2cn3cc(C4CC4)cc(N4CCCC4=O)c3n2)ccn1)[C@@H]1C[C@H]1c1cccc(Cl)c1] rac-(1S*,2S*)-2-(3-chlorophenyl)-N-(4- (((6-cyclopropyl-2-(2-oxopyrrolidin-1- yl)imidazo[1,2-a]pyridin-2- yl)methyl)amino)pyridin-2- yl)cyclopropane-1-carboxamide (I-118) | 1-(2-(((2-chloropyridin-4- yl)amino)methyl)-6- cyclopropylimidazo[1,2-a]pyridin-8- yl)pyrrolidin-2-one ESI-MS (M + H)+: 541.3, ¹H NMR (400 MHz, DMSO) δ 10.36 (s, 1H), 8.30 (d, J = 1.0 Hz, 1H), 7.81 (d, J = 5.8 Hz, 1H), 7.72 (s, 1H), 7.50 (s, 1H), 7.36 (dd, J = 8.0, 8.0 Hz, 1H), 7.30 (d, J = 7.3 Hz, 2H), 7.21-7.17 (m, 3H), 6.37 (dd, J = 1.8, 5.8 Hz, 1H), 4.42 (d, J = 5.6 Hz, 2H), 4.21 (dd, J = 7.1, Hz, 2H), 2.45-2.37 (m, 2H), 2.22- 2.13 (m, 2H), 2.01-1.93 (m, 1H), 1.53- 1.47 (m, 1H), 1.42-1.37 (m, 1H), 1.00-0.94 (m, 2H), 0.72-0.67 (m, 2H). |
| Example 119 | 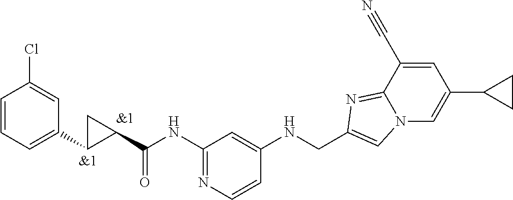 [SMILES: N#Cc1cc(C2CC2)cn2cc(CNc3ccnc(NC(=O)[C@@H]4C[C@H]4c4cccc(Cl)c4)c3)nc12] rac-(1S*,2S*)-2-(3-chlorophenyl)-N-(4- (((8-cyano-6-cyclopropylimidazo[1,2- a]pyridin-2-yl)methyl)amino)pyridin-2- yl)cyclopropane-1-carboxamide (I-119) | 2-(((2-chloropyridin-4- yl)amino)methyl)-6- cyclopropylimidazo[1,2-a]pyridine- 8-carbonitrile ESI-MS (M + H)+: 483.2, ¹H NMR (400 MHz, DMSO) δ 10.37 (s, 1H), 8.72 (d, J = 1.4 Hz, 1H), 7.86 (s, 1H), 7.82 (d, J = 5.9 Hz, 1H), 7.80 (d, J = 1.6 Hz, 1H), 7.49 (s, 1H), 7.37-7.26 (m, 4H), 7.19 (td, J = 1.3, 7.6 Hz, 1H), 6.38 (dd, J = 2.2, 5.8 Hz, 1H), 4.48 (d, J = 6.0 Hz, 2H), 2.46-2.38 (m, 2H), 2.02 (tt, J = 4.0, 9.2 Hz, 1H), 1.50 (ddd, J = 4.0, 5.3, 9.1 Hz, 1H), 1.40 (ddd, J = 4.0, 6.3, 8.0 Hz, 1H), 1.00 (ddd, J = 4.4, 6.5, 8.2 Hz, 2H), 0.80 (td, J = 4.6, 6.2 Hz, 2H). |

### Example 120

### Synthesis of rac-(1S*,2S*)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (I-120)

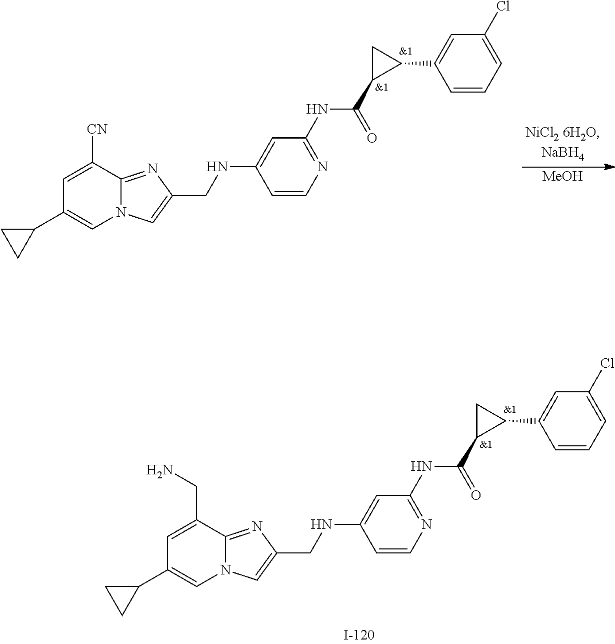 [SMILES: "MeOH" ; N#Cc1cc(C2CC2)cn2cc(CNc3ccnc(NC(=O)[C@H]4C[C@@H]4c4cccc(Cl)c4)c3)nc12 ; NCc1cc(C2CC2)cn2cc(CNc3ccnc(NC(=O)[C@H]4C[C@@H]4c4cccc(Cl)c4)c3)nc12]

To a mixture of rac-(1S*,2S*)-2-(3-chlorophenyl)-N-(4-(((8-cyano-6-cyclopropylimidazo[1,2-a]pyridin-2-yl)methyl)amino)pyridin-2-yl)cyclopropane-1-carboxamide (70 mg, 0.145 mmol), in MeOH (5.0 mL) was added NiCl₂6H₂O (34.6 mg, 0.145 mmol) at 0° C., and then NaBH₄ (15.7 mg, 0.435 mmol) was added. After stirring at 0° C. under N₂ for 1 h, the mixture was quenched with water (1.0 mL), concentrated, and purified by preparative HPLC to give rac-(1S*,2S*)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (6.1 mg, 8.7%) as a white solid. ESI-MS [M+H]+: 487.2. ¹H NMR (400 MHz, DMSO) δ 10.33 (s, 1H), 8.72-7.97 (m, 4H), 7.88-7.58 (m, 2H), 7.45 (s, 1H), 7.37-6.95 (m, 6H), 6.32 (s, 1H), 4.38 (s, 2H), 4.14 (s, 2H), 2.39-2.33 (m, 2H), 1.93-1.87 (m, 1H), 1.45-1.41 (m, 1H), 1.36-1.31 (m, 1H), 0.93-0.88 (m, 2H), 0.80-0.55 (m, 2H).

### Synthesis of 2-(2-(((2-bromopyridin-4-yl)amino)methyl)-6-cyclopropylimidazo[1,2-a]pyridin-8-yl)isothiazolidine 1,1-dioxide

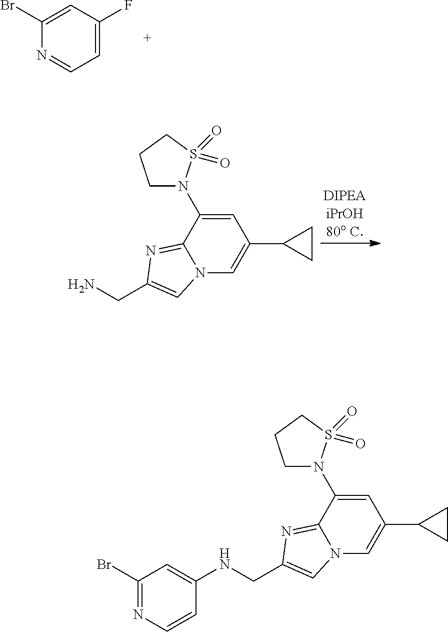 [SMILES: Fc1ccnc(Br)c1 ; NCc1cn2cc(C3CC3)cc(N3CCCS3(=O)=O)c2n1 ; O=S1(=O)CCCN1c1cc(C2CC2)cn2cc(CNc3ccnc(Br)c3)nc12]

A mixture of 2-(2-(aminomethyl)-6-cyclopropylimidazo[1,2-a]pyridin-8-yl)isothiazolidine 1,1-dioxide (0.18 g, 0.59 mmol), 2-bromo-4-fluoropyridine (0.098 mL, 0.56 mmol) and DIPEA (0.36 mL, 2.1 mmol) in iPrOH (5.0 mL) was stirred at 80° C. for 18 h. The mixture was concentrated in vacuo. The residue was purified by column chromatography on silica gel, eluting with 0-100% EtOAc in cyclohexane to give the title compound. ¹H NMR (400 MHz, CDCl₃) δ, 7.76-7.74 (m, 1H), 7.41-7.39 (m, 1H), 7.15 (d, J=1.5 Hz, 1H), 6.99-6.95 (m, 1H), 6.78 (d, J=2.1 Hz, 1H), 6.48-6.45 (m, 1H), 4.45 (dd, J=5.3, 8.3 Hz, 2H), 4.31 (t, J=6.8 Hz, 2H), 3.41 (t, J=7.5 Hz, 2H), 2.66-2.59 (m, 2H), 1.91-1.84 (m, 1H), 1.00-0.93 (m, 2H), 0.71-0.67 (m, 2H).

The compounds in Table 17 were synthesized using a similar procedure to that used for 2-(2-(((2-bromopyridin-4-yl)amino)methyl)-6-cyclopropylimidazo[1,2-a]pyridin-8-yl)isothiazolidine 1,1-dioxide from 2-bromo-4-fluoropyridine and an appropriate coupling partner.

TABLE 17

| | Coupling Partner/ |
|---|---|
| Compound | Analytical Data |
| 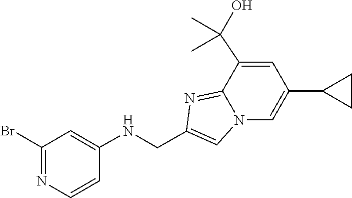 [SMILES: CC(C)(O)c1cc(C2CC2)cn2cc(CNc3ccnc(Br)c3)nc12] | 2-(2-(aminomethyl)-6-cyclopropylimidazo[1,2- a]pyridin-8-yl)propan-2-ol ¹H NMR (400 MHz, CDCl₃) δ 7.94 (d, J = 5.8 Hz, 1H), 7.74 (s, 1H), 7.36 (s, 1H), 6.85 (d, J = 1.5 Hz, 1H), 6.72 (d, J = 2.1 Hz, 1H), 6.48 (dd, J = 2.3, 5.8 Hz, 1H), 6.31 (s, 1H), 4.95 (dd, J = 4.6, 4.6 Hz, 1H), 4.46 (d, J = 5.1 Hz, 2H), 1.92-1.85 (m, 1H), 1.70 (s, 6H), 0.99-0.93 (m, 2H), 0.69-0.63 (m, 2H). |
| 2-(2-(((2-bromopyridin-4-yl)amino)methyl)-6- | |
| cyclopropylimidazo[1,2-a]pyridin-8-yl)propan- | |
| 2-ol | |
| 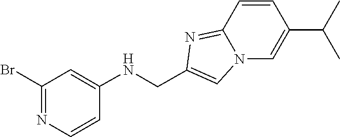 [SMILES: CC(C)c1ccc2nc(CNc3ccnc(Br)c3)cn2c1] | (6-isopropylimidazo[1,2-a]pyridin-2- yl)methanamine ESI-MS (M + H)+: 347.0, 345.0 ¹H NMR (400 MHz, DMSO) δ 8.35 (d, J = 0.4 Hz, 1H), 7.78 (d, J = 5.8 Hz, 1H), 7.74 (s, 1H), 7.46-7.42 (m, 2H), 7.21 (dd, J = 1.8, 9.3 Hz, 1H), 6.74 (s, 1H), 6.62 (dd, J = 2.1, 5.8 Hz, 1H), 4.40 (d, J = 5.8 Hz, 2H), 2.94-2.86 (m, 1H), 1.24 (d, J = 6.9 Hz, 6H). |
| 2-bromo-N-((6-isopropylimidazo[1,2- | |
| a]pyridin-2-yl)methyl)pyridin-4-amine | |
| 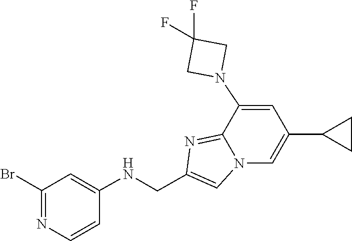 [SMILES: FC1(F)CN(c2cc(C3CC3)cn3cc(CNc4ccnc(Br)c4)nc23)C1] | (6-cyclopropyl-8-(3,3-difluoroazetidin-1- yl)imidazo[1,2-a]pyridin-2-yl)methanamine ESI-MS (M + H)+: 434.1, ¹H NMR (400 MHz, DMSO) δ 7.88 (s, 1H), 7.83 (d, J = 5.8 Hz, 1H), 7.67 (s, 1H), 7.42-7.37 (m, 1H), 6.87 (d, J = 2.0 Hz, 1H), 6.66 (dd, J = 2.0, 5.8 Hz, 1H), 5.93 (s, 1H), 4.58 (dd, J = 12.4, 12.4 Hz, 4H), 4.40 (dd, J = 5.8, 5.8 Hz, 2H), 1.93-1.85 (m, 1H), 0.96- 0.89 (m, 2H), 0.74-0.68 (m, 2H). |
| 2-bromo-N-((6-cyclopropyl-8-(3,3- | |
| difluoroazetidin-1-yl)imidazo[1,2-a]pyridin-2- | |
| yl)methyl)pyridin-4-amine | |
| 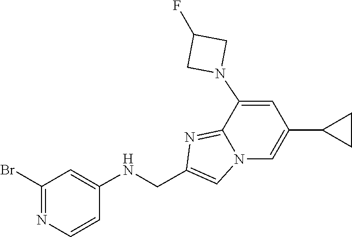 [SMILES: FC1CN(c2cc(C3CC3)cn3cc(CNc4ccnc(Br)c4)nc23)C1] | (6-cyclopropyl-8-(3-fluoroazetidin-1- yl)imidazo[1,2-a]pyridin-2-yl)methanamine ESI-MS (M + H)+: 416.1, ¹H NMR (400 MHz, DMSO) δ 7.79 (d, J = 5.7 Hz, 1H), 7.77-7.75 (m, 1H), 7.60 (s, 1H), 7.40-7.33 (m, 1H), 6.82 (d, J = 2.0 Hz, 1H), 6.62 (dd, J = 2.1, 5.8 Hz, 1H), 5.75 (d, J = 1.5 Hz, 1H), 5.59-5.40 (m, 1H), 4.46 (ddd, J = 5.0, 10.4, 20.8 Hz, 2H), 4.35 (d, J = 5.6 Hz, 2H), 4.21-4.10 (m, 2H), 1.88-180 (m, 1H), 0.89-0.84 (m, 2H), 0.68-0.63 (m, 2H). |
| 2-bromo-N-((6-cyclopropyl-8-(3- | |
| fluoroazetidin-1-yl)imidazo[1,2-a]pyridin-2- | |
| yl)methyl)pyridin-4-amine | |
| 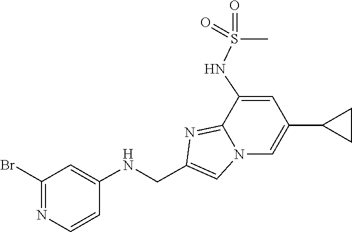 [SMILES: CS(=O)(=O)Nc1cc(C2CC2)cn2cc(CNc3ccnc(Br)c3)nc12] | N-(2-(aminomethyl)-6-cyclopropylimidazo[1,2- a]pyridin-8-yl)methanesulfonamide ESI-MS (M + H)+: 438.3 |
| N-(2-(((2-bromopyridin-4-yl)amino)methyl)-6- | |
| cyclopropylimidazo[1,2-a]pyridin-8- | |
| yl)methanesulfonamide | |
| 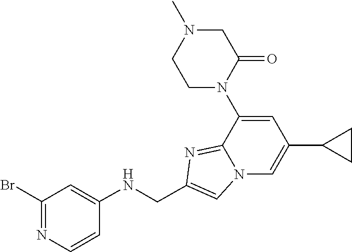 [SMILES: CN1CCN(c2cc(C3CC3)cn3cc(CNc4ccnc(Br)c4)nc23)C(=O)C1] | 1-(2-(aminomethyl)-6-cyclopropylimidazo[1,2- a]pyridin-8-yl)-4-methylpiperazin-2-one ¹H NMR (400 MHz, CDCl₃) δ 7.92 (d, J = 5.8 Hz, 1H), 7.83 (s, 1H), 7.42 (s, 1H), 7.00-6.97 (m, 1H), 6.72 (d, J = 2.1 Hz, 1H), 6.47 (dd, J = 2.3, 5.8 Hz, 1H), 4.45 (d, J = 5.1 Hz, 2H), 3.92 (dd, J = 5.5, 5.5 Hz, 2H), 3.37 (s, 2H), 2.91 (dd, J = 5.5, 5.5 Hz, 2H), 2.45 (s, 3H), 1.91-1.85 (m, 1H), 0.99- 0.94 (m, 2H), 0.70-0.66 (m, 2H). |
| 1-(2-(((2-bromopyridin-4-yl)amino)methyl)-6- | |
| cyclopropylimidazo[1,2-a]pyridin-8-yl)-4- | |
| methylpiperazin-2-one | |
| 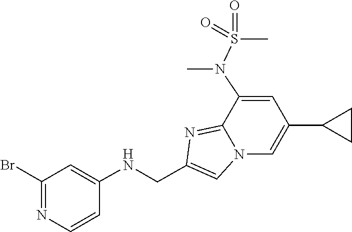 [SMILES: CN(c1cc(C2CC2)cn2cc(CNc3ccnc(Br)c3)nc12)S(C)(=O)=O] | N-(2-(aminomethyl)-6-cyclopropylimidazo[1,2- a]pyridin-8-yl)-N-methylmethanesulfonamide ESI-MS (M +H)⁺: 452.0, ¹H NMR (400 MHz, CDCl₃) δ 7.92 (d, J = 5.6 Hz, 1H), 7.84 (s, 1H), 7.43 (s, 1H), 7.08 (d, J = 1.5 Hz, 1H), 6.76 (d, J = 2.3 Hz, 1H), 6.46 (dd, J = 2.3, 5.8 Hz, 1H), 5.06 (dd, J = 5.2, 5.2 Hz, 1H), 4.46 (d, J = 5.6 Hz, 2H), 3.51 (s, 3H), 3.13 (s, 3H), 1.93-1.83 (m, 2H), 1.01-0.94 (m, 2H), 0.71-0.65 (m, 2H). |
| N-(2-(((2-bromopyridin-4-yl)amino)methyl)-6- | |
| cyclopropylimidazo[1,2-a]pyridin-8-yl)-N- | |
| methylmethanesulfonamide | |
| 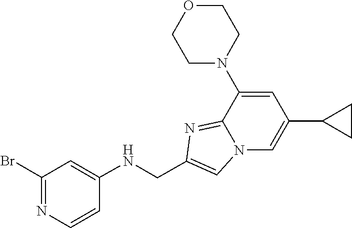 [SMILES: Brc1cc(NCc2cn3cc(C4CC4)cc(N4CCOCC4)c3n2)ccn1] | (6-cyclopropyl-8-morpholinoimidazo[1,2- a]pyridin-2-yl)methanamine ESI-MS (M + H)+: 430.1, 1H NMR (400 MHz, CDCl₃) δ 7.91 (d, J = 5.6 Hz, 1H), 7.52 (s, 1H), 7.32 (s, 1H), 6.73 (d, J = 2.0 Hz, 1H), 6.46 (dd, J = 2.0, 5.8 Hz, 1H), 6.24 (d, J = 1.3 Hz, 1H), 5.17 (dd, J = 5.1, 5.1 Hz, 1H), 4.43 (d, J = 5.3 Hz, 2H), 3.99-3.95 (m, 4H), 3.49 (dd, J = 4.7, 4.7 Hz, 4H), 1.89-1.81 (m, 1H), 0.96-0.87 (m, 2H), 0.68- 0.63 (m, 2H). |
| 2-bromo-N-((6-cyclopropyl-8- | |
| morpholinoimidazo[1,2-a]pyridin-2- | |
| yl)methyl)pyridin-4-amine | |
| 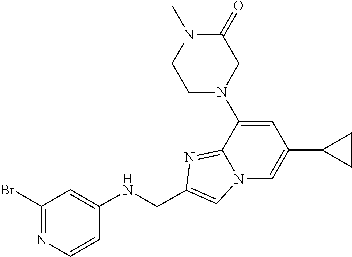 [SMILES: CN1CCN(c2cc(C3CC3)cn3cc(CNc4ccnc(Br)c4)nc23)CC1=O] | 4-(2-(aminomethyl)-6-cyclopropylimidazo[1,2- a]pyridin-8-yl)-1-methylpiperazin-2-one ESI-MS (M + H)+: 455.3, 457.3 |
| 4-(2-(((2-bromopyridin-4-yl)amino)methyl)-6- | |
| cyclopropylimidazo[1,2-a]pyridin-8-yl)-1- | |
| methylpiperazin-2-one | |

### Example 121

### Synthesis of (1S,2S)-2-(3-chlorophenyl)-N-(4-(((6-cyclopropylimidazo[1,2-a]pyridin-2-yl)methyl)amino)pyridin-2-yl)cyclopropane-1-carboxamide (I-121)

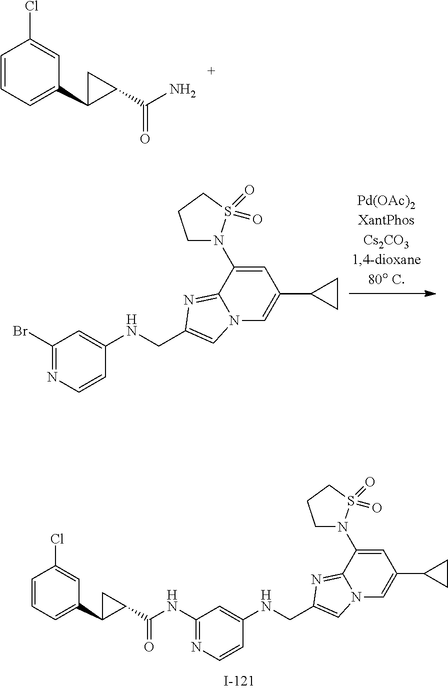 [SMILES: NC(=O)[C@H]1C[C@@H]1c1cccc(Cl)c1 ; O=S1(=O)CCCN1c1cc(C2CC2)cn2cc(CNc3ccnc(Br)c3)nc12 ; O=C(Nc1cc(NCc2cn3cc(C4CC4)cc(N4CCCS4(=O)=O)c3n2)ccn1)[C@H]1C[C@@H]1c1cccc(Cl)c1]

A mixture of 2-(2-(((2-bromopyridin-4-yl)amino)methyl)-6-cyclopropylimidazo[1,2-a]pyridin-8-yl)isothiazolidine 1,1-dioxide (0.090 g, 0.20 mmol), (1S,2S)-2-(3-chlorophenyl)cyclopropane-1-carboxamide (0.042 g, 0.21 mmol), Pd(OAc)₂ (0.009 g, 0.039 mmol), XantPhos (0.045 g, 0.078 mmol), and Cs₂CO₃ (0.13 g, 0.39 mmol) in 1,4-dioxane (5.0 mL) was degassed with nitrogen then stirred at 80° C. for 18 h. The mixture was filtered through Celite® and concentrated in vacuo. The residue was purified by preparative HPLC to give the title compound (0.016 g, 14%). ESI-MS (M+H)+: 577, ¹H NMR (400 MHz, DMSO) δ 10.38-10.38 (m, 1H), 8.26 (s, 1H), 7.78 (d, J=5.9 Hz, 1H), 7.69 (s, 1H), 7.46 (s, 1H), 7.32 (dd, J=7.7, 7.7 Hz, 1H), 7.26 (d, J=7.4 Hz, 2H), 7.15 (d, J=7.9 Hz, 2H), 6.97 (s, 1H), 6.34 (d, J=3.9 Hz, 1H), 4.39 (d, J=5.3 Hz, 2H), 4.25 (dd, J=6.7, 6.7 Hz, 2H), 3.53-3.47 (m, 2H), 2.49-2.44 (m, 2H), 2.37 (d, J=6.5 Hz, 2H), 1.97-1.89 (m, 1H), 1.47-1.43 (m, 1H), 1.40-1.35 (m, 1H), 0.94 (q, J=6.1 Hz, 2H), 0.65-0.61 (m, 2H).

Using a similar procedure to that used for (1S,2S)-2-(3-chlorophenyl)-N-(4-(((6-cyclopropylimidazo[1,2-a]pyridin-2-yl)methyl)amino)pyridin-2-yl)cyclopropane-1-carboxamide, the compounds in Table 18 were prepared from (1S,2S)-2-(3-chlorophenyl)cyclopropane-1-carboxamide and an appropriate coupling partner, then purified by preparative LCMS.

TABLE 18

| | | Coupling Partner/ |
|---|---|---|
| Example | Compound | Analytical Data |
| Example 122 | 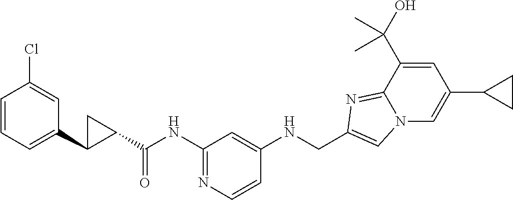 [SMILES: CC(C)(O)c1cc(C2CC2)cn2cc(CNc3ccnc(NC(=O)[C@H]4C[C@@H]4c4cccc(Cl)c4)c3)nc12] (1S,2S)-2-(3-chlorophenyl)-N-(4-(((6- cyclopropyl-8-(2-hydroxypropan-2- yl)imidazo[1,2-a]pyridin-2- yl)methyl)amino)pyridin-2- yl)cyclopropane-1-carboxamide (I-122) | 2-(2-(((2-bromopyridin-4- yl)amino)methyl)-6- cyclopropylimidazo[1,2-a]pyridin-8- yl)propan-2-ol ESI-MS (M + H)+: 516.4, ¹H NMR (400 MHz, DMSO) δ 10.35 (s, 1H), 8.19 (d, J = 1.4 Hz, 1H), 7.77 (d, J = 5.8 Hz, 1H), 7.61 (s, 1H), 7.47-7.46 (m, 1H), 7.33 (dd, J = 7.7, 7.7 Hz, 1H), 7.26 (d, J = 8.9 Hz, 2H), 7.15 (d, J = 7.7 Hz, 2H), 7.07 (d, J = 1.6 Hz, 1H), 6.35 (dd, J = 2.0, 5.8 Hz, 1H), 5.52 (s, 1H), 4.38 (d, J = 5.8 Hz, 2H), 2.41-2.34 (m, 2H), 1.96-1.88 (m, 1H), 1.67 (s, 6H), 1.48-1.33 (m, 2H), 0.95-0.89 (m, 2H), 0.69-0.64 (m, 2H). |
| Example 123 | 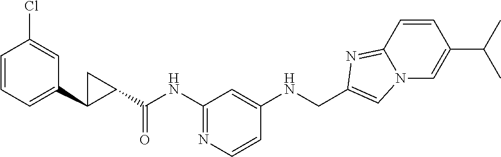 [SMILES: CC(C)c1ccc2nc(CNc3ccnc(NC(=O)[C@H]4C[C@@H]4c4cccc(Cl)c4)c3)cn2c1] (1S,2S)-2-(3-chlorophenyl)-N-(4-(((6- isopropylimidazo[1,2-a]pyridin-2- yl)methyl)amino)pyridin-2- yl)cyclopropane-1-carboxamide (I-123) | 2-bromo-N-((6-isopropylimidazo[1,2- a]pyridin-2-yl)methyl)pyridin-4-amine ESI-MS (M + H)+: 460.4, ¹H NMR (400 MHz, DMSO) δ 10.31 (s, 1H), 8.32- 8.31 (m, 1H), 7.75 (d, J = 5.8 Hz, 1H), 7.66 (s, 1H), 7.44-7.40 (m, 2H), 7.33- 7.28 (m, 1H), 7.26-7.23 (m, 2H), 7.20- 7.12 (m, 3H), 6.32 (dd, J = 2.1, 5.8 Hz, 1H), 4.36 (d, J = 5.8 Hz, 2H), 2.93-2.82 (m, 1H), 2.40-2.31 (m, 2H), 1.47-1.41 (m, 1H), 1.37-1.31 (m, 1H), 1.22 (d, J = 6.9 Hz, 6H). |
| Example 124 | 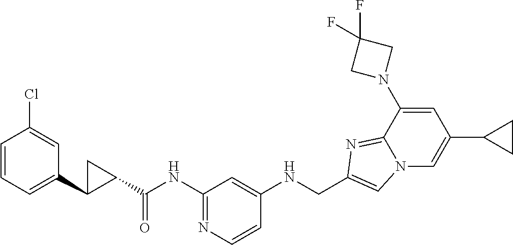 [SMILES: O=C(Nc1cc(NCc2cn3cc(C4CC4)cc(N4CC(F)(F)C4)c3n2)ccn1)[C@H]1C[C@@H]1c1cccc(Cl)c1] | 2-bromo-N-((6-cyclopropyl-8-(3,3- difluoroazetidin-1-yl)imidazo[1,2- a]pyridin-2-yl)methyl)pyridin-4-amine ESI-MS (M + H)+: 549.3, ¹H NMR (400 MHz, DMSO) δ 10.34 (br s, 1H), 7.82- 7.80 (m, 1H), 7.75 (d, J = 5.9 Hz, 1H), 7.57 (s, 1H), 7.44-7.23 (m, 4H), 7.16- 7.09 (m, 2H), 6.34-6.32 (m, 1H), 5.86 (d, J = 1.0 Hz, 1H), 4.53 (t, J = 12.3 Hz, 4H), 4.32 (d, J = 5.6 Hz, 2H), 2.39-2.31 (m, 2H), 1.87-1.79 (m, 1H), 1.48-1.41 (m, 1H), 1.38-1.31 (m, 1H), 0.89-0.83 (m, 2H), 0.69-0.63 (m, 2H). |
| | (1S,2S)-2-(3-chlorophenyl)-N-(4-(((6- | |
| | cyclopropyl-8-(3,3-difluoroazetidin-1- | |
| | yl)imidazo[1,2-a]pyridin-2- | |
| | yl)methyl)amino)pyridin-2- | |
| | yl)cyclopropane-1-carboxamide (I-124) | |
| Example 125 | 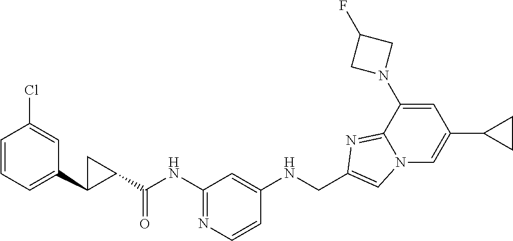 [SMILES: O=C(Nc1cc(NCc2cn3cc(C4CC4)cc(N4CC(F)C4)c3n2)ccn1)[C@H]1C[C@@H]1c1cccc(Cl)c1] | 2-bromo-N-((6-cyclopropyl-8-(3- fluoroazetidin-1-yl)imidazo[1,2- a]pyridin-2-yl)methyl)pyridin-4-amine ESI-MS (M + H)+: 531.3, ¹H NMR (400 MHz, DMSO) δ 10.30 (s, 1H), 7.76- 7.73 (m, 2H), 7.52 (s, 1H), 7.45 (br s, 1H), 7.31-7.23 (m, 3H), 7.15-7.12 (m, 1H), 7.05 (t, J = 5.8 Hz, 1H), 6.32 (dd, J = 2.1, 5.8 Hz, 1H), 5.73 (d, J = 1.2 Hz, 1H), 5.57-5.37 (m, 1H), 4.51-4.40 (m, 2H), 4.33-4.29 (m, 2H), 4.19-4.08 (m, 2H), 2.40-2.32 (m, 2H), 1.85-1.77 (m, 1H), 1.47-1.41 (m, 1H), 1.37-1.31 (m, 1H), 0.87-0.81 (m, 2H), 0.66-0.61 (m, 2H). |
| | (1S,2S)-2-(3-chlorophenyl)-N-(4-(((6- | |
| | cyclopropyl-8-(3-fluoroazetidin-1- | |
| | yl)imidazo[1,2-a]pyridin-2- | |
| | yl)methyl)amino)pyridin-2- | |
| | yl)cyclopropane-1-carboxamide (I-125) | |
| Example 126 | 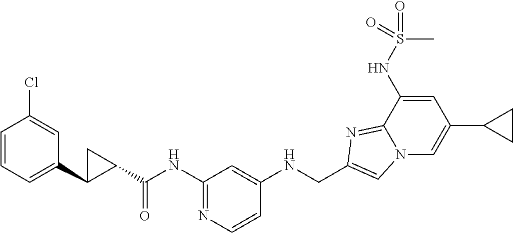 [SMILES: CS(=O)(=O)Nc1cc(C2CC2)cn2cc(CNc3ccnc(NC(=O)[C@H]4C[C@@H]4c4cccc(Cl)c4)c3)nc12] | N-(2-(((2-bromopyridin-4- yl)amino)methyl)-6- cyclopropylimidazo[1,2-a]pyridin-8- yl)methanesulfonamide ESI-MS (M + H)+: 551, ¹H NMR (400 MHz, DMSO) δ 10.37 (s, 1H), 8.42 (s, 1H), 8.06 (s, 1H), 7.81 (d, J = 5.8 Hz, 1H), 7.67 (s, 1H), 7.50 (s, 1H), 7.37- 7.28 (m, 3H), 7.21-7.11 (m, 2H), 6.85 (s, 1H), 6.37 (dd, J = 2.0, 5.8 Hz, 1H), 4.42 (d, J = 7.1 Hz, 2H), 3.16 (s, 3H), 2.46-2.37 (m, 2H), 1.97-1.90 (m, 1H), 1.53-1.47 (m, 1H), 1.43-1.36 (m, 1H), 0.98-0.92 (m, 2H), 0.70-0.65 (m, 2H). |
| | (1S,2S)-2-(3-chlorophenyl)-N-(4-(((6- | |
| | cyclopropyl-8- | |
| | (methylsulfonamido)imidazo[1,2- | |
| | a]pyridin-2-yl)methyl)amino)pyridin-2- | |
| | yl)cyclopropane-1-carboxamide (I-126) | |
| Example 127 | 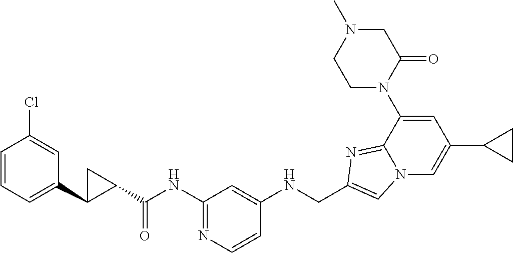 [SMILES: CN1CCN(c2cc(C3CC3)cn3cc(CNc4ccnc(NC(=O)[C@H]5C[C@@H]5c5cccc(Cl)c5)c4)nc23)C(=O)C1] | 1-(2-(((2-bromopyridin-4- yl)amino)methyl)-6- cyclopropylimidazo[1,2-a]pyridin-8- yl)-4-methylpiperazin-2-one ESI-MS (M + H)+: 570.4, ¹H NMR (400 MHz, DMSO) δ 10.18 (s, 1H), 8.18 (d, J = 1.0 Hz, 1H), 7.63 (d, J = 5.8 Hz, 1H), 7.54 (s, 1H), 7.31 (s, 1H), 7.21-7.16 (m, 1H), 7.13-7.11 (m, 2H), 7.04-6.99 (m, 2H), 6.82 (d, J = 1.8 Hz, 1H), 6.18 (dd, J = 2.0, 5.8 Hz, 1H), 4.23 (d, J = 5.8 Hz, 2H), 3.64 (dd, J = 5.6, 5.6 Hz, 2H), 3.02 (s, 2H), 2.64 (dd, J = 5.4, 5.4 Hz, 2H), 2.27-2.21 (m, 2H), 2.20 (s, 3H), 1.83- 1.75 (m, 1H), 1.34-1.19 (m, 2H), 0.82- 0.76 (m, 2H), 0.57-0.52 (m, 2H). |
| | (1S,2S)-2-(3-chlorophenyl)-N-(4-(((6- | |
| | cyclopropyl-8-(4-methyl-2- | |
| | oxopiperazin-1-yl)imidazo[1,2- | |
| | a]pyridin-2-yl)methyl)amino)pyridin-2- | |
| | yl)cyclopropane-1-carboxamide (I-127) | |
| Example 128 | 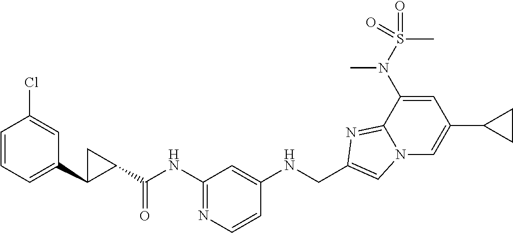 [SMILES: CN(c1cc(C2CC2)cn2cc(CNc3ccnc(NC(=O)[C@H]4C[C@@H]4c4cccc(Cl)c4)c3)nc12)S(C)(=O)=O] | N-(2-(((2-bromopyridin-4- yl)amino)methyl)-6- cyclopropylimidazo[1,2-a]pyridin-8- yl)-N-methylmethanesulfonamide ESI-MS (M + H)+: 565, ¹H NMR (400 MHz, DMSO) δ 10.23 (s, 1H), 8.27- 8.25 (m, 1H), 7.70-7.63 (m, 2H), 7.41- 7.38 (m, 1H), 7.27-7.15 (m, 3H), 7.09- 7.03 (m, 2H), 6.95 (d, J = 1.5 Hz, 1H), 6.26 (dd, J = 2.0, 5.8 Hz, 1H), 4.31 (d, J = 5.8 Hz, 2H), 3.31 (s, 3H), 3.16-3.14 (m, 3H), 2.32-2.25 (m, 2H), 1.90-1.82 (m, 1H), 1.40-1.24 (m, 2H), 0.89-0.82 (m, 2H), 0.63-0.58 (m, 2H). |
| | (1S,2S)-2-(3-chlorophenyl)-N-(4-(((6- | |
| | cyclopropyl-8-(N- | |
| | methylmethylsulfonamido)imidazo[1,2- | |
| | a]pyridin-2-yl)methyl)amino)pyridin-2- | |
| | yl)cyclopropane-1-carboxamide, formic | |
| | acid salt (I-128) | |
| Example 129 | 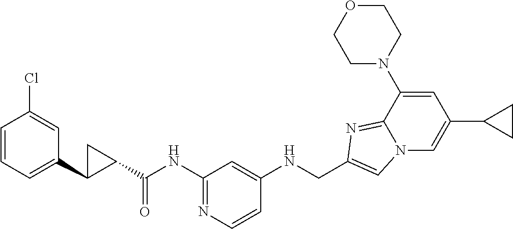 [SMILES: O=C(Nc1cc(NCc2cn3cc(C4CC4)cc(N4CCOCC4)c3n2)ccn1)[C@H]1C[C@@H]1c1cccc(Cl)c1] | 2-bromo-N-((6-cyclopropyl-8- morpholinoimidazo[1,2-a]pyridin-2- yl)methyl)pyridin-4-amine ESI-MS (M + H)+: 543.4, ¹H NMR (400 MHz, DMSO) δ 10.23 (s, 1H), 7.82 (s, 1H), 7.69 (d, J = 5.8 Hz, 1H), 7.49 (s, 1H), 7.37 (s, 1H), 7.25-7.16 (m, 3H), 7.09-7.00 (m, 2H), 6.25 (dd, J = 2.3, 5.8 Hz, 1H), 6.11-6.10 (m, 1H), 4.29-4.25 (m, 2H), 3.75-3.70 (m, 4H), 3.44-3.38 (m, 4H), 2.34-2.26 (m, 2H), 1.83-1.75 (m, 1H), 1.41-1.24 (m, 2H), 0.83-0.77 (m, 2H), 0.62-0.57 (m, 2H). |
| | (1S,2S)-2-(3-chlorophenyl)-N-(4-(((6- | |
| | cyclopropyl-8-morpholinoimidazo[1,2- | |
| | a]pyridin-2-yl)methyl)amino)pyridin-2- | |
| | yl)cyclopropane-1-carboxamide (I-129) | |
| Example 130 | 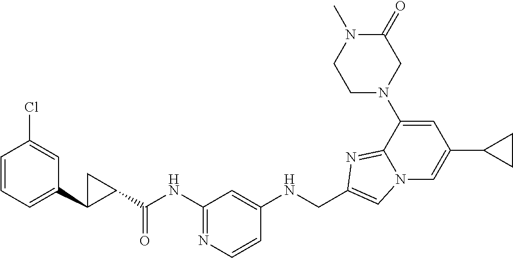 [SMILES: CN1CCN(c2cc(C3CC3)cn3cc(CNc4ccnc(NC(=O)[C@H]5C[C@@H]5c5cccc(Cl)c5)c4)nc23)CC1=O] (1S,2S)-2-(3-chlorophenyl)-N-(4-(((6- cyclopropyl-8-(4-methyl-3- oxopiperazin-1-yl)imidazo[1,2- a]pyridin-2-yl)methyl)amino)pyridin-2- yl)cyclopropane-1-carboxamide (I-130) | 4-(2-(((2-bromopyridin-4- yl)amino)methyl)-6- cyclopropylimidazo[1,2-a]pyridin-8- yl)-1-methylpiperazin-2-one ESI-MS (M + H)+: 570.4, ¹H NMR (400 MHz, DMSO) δ 10.30 (s, 1H), 7.91- 7.90 (m, 1H), 7.75 (d, J = 5.8 Hz, 1H), 7.57 (s, 1H), 7.48-7.42 (m, 1H), 7.32- 7.28 (m, 1H), 7.26-7.23 (m, 2H), 7.16- 7.07 (m, 2H), 6.32 (dd, J = 2.0, 5.8 Hz, 1H), 6.19 (d, J = 1.3 Hz, 1H), 4.35 (d, J = 5.8 Hz, 2H), 4.07 (s, 2H), 3.92-3.86 (m, 2H), 3.46 (dd, J = 5.5, 5.5 Hz, 2H), 2.91 (s, 3H), 2.40-2.32 (m, 2H), 1.90- 1.82 (m, 1H), 1.47-1.41 (m, 1H), 1.37- 1.31 (m, 1H), 0.90-0.83 (m, 2H), 0.71- 0.65 (m, 2H). |

Using a similar procedure to that used for (1S,2S)-2-(3-chlorophenyl)-N-(4-(((6-cyclopropylimidazo[1,2-a]pyridin-2-yl)methyl)amino)pyridin-2-yl)cyclopropane-1-carboxamide, the compound in Table 19 was prepared using rac-(1S*,2S*)-2-(5-chloro-2-cyanophenyl)cyclopropane-1-carboxamide and an appropriate coupling partner, then purified by preparative LCMS.

TABLE 19

| | | Coupling Partner/ |
|---|---|---|
| Example | Compound | Analytical Data |
| Example 131 | 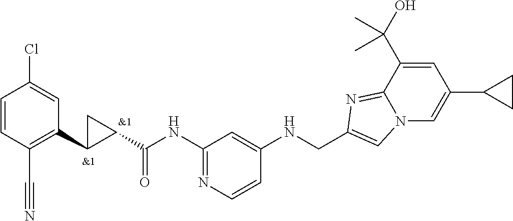 [SMILES: CC(C)(O)c1cc(C2CC2)cn2cc(CNc3ccnc(NC(=O)[C@H]4C[C@@H]4c4cc(Cl)ccc4C#N)c3)nc12] rac-(1S*,2S*)-2-(5-chloro-2-cyanophenyl)- N-(4-(((6-cyclopropyl-8-(2- hydroxypropan-2-yl)imidazo[1,2- a]pyridin-2-yl)methyl)amino)pyridin-2- yl)cyclopropane-1-carboxamide (I-131) | 2-(2-(((2-bromopyridin-4- yl)amino)methyl)-6- cyclopropylimidazo[1,2-a]pyridin-8- yl)propan-2-ol ESI-MS (M + H)+: 541.3, ¹H NMR (400 MHz, DMSO) δ 10.44 (s, 1H), 8.26 (d, J = 1.3 Hz, 1H), 7.95 (d, J = 8.3 Hz, 1H), 7.86 (d, J = 5.8 Hz, 1H), 7.69 (s, 1H), 7.62-7.55 (m, 2H), 7.47 (d, J = 2.0 Hz, 1H), 7.21 (dd, J = 5.8, 5.8 Hz, 1H), 7.14 (d, J = 1.8 Hz, 1H), 6.43 (dd, J = 2.0, 5.8 Hz, 1H), 5.59 (s, 1H), 4.46 (d, J = 5.6 Hz, 2H), 2.69-2.63 (m, 1H), 2.03-1.95 (m, 1H), 1.74 (s, 6H), 1.65-1.60 (m, 2H), 0.99 (ddd, J = 4.3, 6.3, 8.3 Hz, 2H), 0.76-0.71 (m, 2H). |

### Synthesis of tert-butyl 4-(2-(((2-bromopyridin-4-yl)amino)methyl)-6-cyclopropylimidazo[1,2-a]pyridin-8-yl)piperazine-1-carboxylate

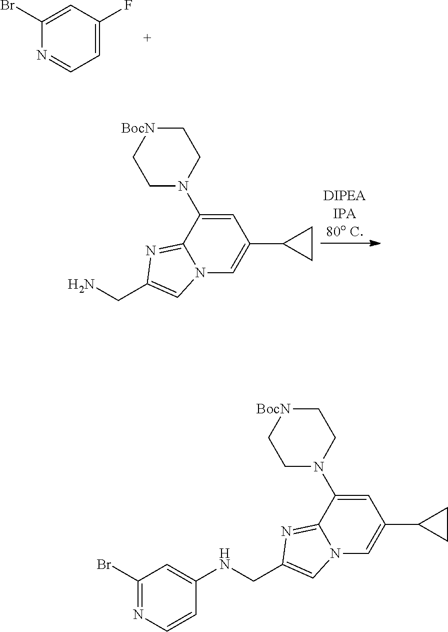 [SMILES: Fc1ccnc(Br)c1 ; CC(C)(C)OC(=O)N1CCN(c2cc(C3CC3)cn3cc(CN)nc23)CC1 ; CC(C)(C)OC(=O)N1CCN(c2cc(C3CC3)cn3cc(CNc4ccnc(Br)c4)nc23)CC1]

A mixture of tert-butyl 4-(2-(aminomethyl)-6-cyclopropylimidazo[1,2-a]pyridin-8-yl)piperazine-1-carboxylate (0.30 g, 0.80 mmol), 2-bromo-4-fluoropyridine (0.15 g, 0.84 mmol), and DIPEA (0.28 mL, 1.6 mmol) in iPrOH (5.0 mL) was stirred at 80° C. for 18 h. The mixture was concentrated in vacuo and the residue was purified by column chromatography on silica gel, eluting with 0-100% ethyl acetate in cyclohexane to give the title compound (0.17 g, 40%). ¹H NMR (400 MHz, CDCl₃) δ 7.93 (d, J=5.8 Hz, 1H), 7.53-7.52 (m, 1H), 7.33-7.32 (m, 1H), 6.73 (d, J=2.1 Hz, 1H), 6.46 (dd, J=2.3, 5.8 Hz, 1H), 6.24-6.23 (7, 1H), 5.03-5.00 (m, 1H), 4.44 (d, J=5.1 Hz, 2H), 3.72-3.68 (m, 4H), 3.47-3.41 (m, 4H), 1.88-1.80 (m, 1H), 1.50 (s, 9H), 0.96-0.90 (m, 2H), 0.67-0.62 (m, 2H).

The compounds in Table 20 were synthesized using a similar procedure to that used for tert-butyl 4-(2-(((2-bromopyridin-4-yl)amino)methyl)-6-cyclopropylimidazo[1,2-a]pyridin-8-yl)piperazine-1-carboxylate from 2-bromo-4-fluoropyridine and an appropriate coupling partner.

TABLE 20

| | Coupling Partner/ |
|---|---|
| Compound | Analytical Data |
| 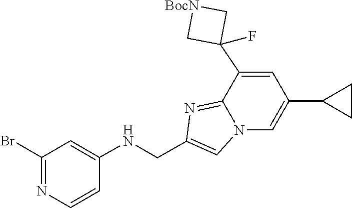 [SMILES: CC(C)(C)OC(=O)N1CC(F)(c2cc(C3CC3)cn3cc(CNc4ccnc(Br)c4)nc23)C1] | tert-butyl 3-(2-(aminomethyl)-6- cyclopropylimidazo[1,2-a]pyridin-8-yl)-3- fluoroazetidine-1-carboxylate ¹H NMR (400 MHz, CDCl₃) δ 7.93 (d, J = 5.8 Hz, 1H), 7.86 (s, 1H), 7.43 (s, 1H), 7.03 (d, J = 1.6 Hz, 1H), 6.75 (d, J = 2.1 Hz, 1H), 6.59 (dd, J = 2.2, 5.8 Hz, 1H), 5.51 (dd, J = 4.8, 4.8 Hz, 1H), 4.87-4.87 (m, 2H), 4.40-4.37 (m, 4H), 4.32 (s, 4H), 1.94-1.87 (m, 1H), 1.45 (s, 9H), 1.01-0.96 (m, 2H), 0.71-0.66 (m, 2H). |
| tert-butyl 3-(2-(((2-bromopyridin-4- | |
| yl)amino)methyl)-6-cyclopropylimidazo[1,2- | |
| a]pyridin-8-yl)-3-fluoroazetidine-1-carboxylate | |
| 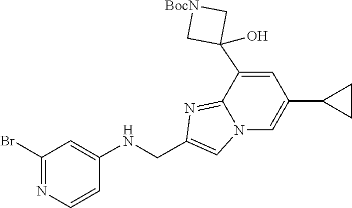 [SMILES: CC(C)(C)OC(=O)N1CC(O)(c2cc(C3CC3)cn3cc(CNc4ccnc(Br)c4)nc23)C1] | tert-butyl 3-(2-(aminomethyl)-6- cyclopropylimidazo[1,2-a]pyridin-8-yl)-3- hydroxyazetidine-1-carboxylate ¹H NMR (400 MHz, CDCl₃) δ 7.94 (d, J = 5.8 Hz, 1H), 7.79 (s, 1H), 7.40 (s, 1H), 7.06 (d, J = 1.5 Hz, 1H), 6.89 (s, 1H), 6.72 (d, J = 2.1 Hz, 1H), 6.48 (dd, J = 2.2, 5.8 Hz, 1H), 4.92 (dd, J = 5.2, 5.2 Hz, 1H), 4.46 (d, J = 5.4 Hz, 2H), 4.32-4.25 (m, 4H), 1.96-1.89 (m, 1H), 1.48 (s, 9H), 1.03-0.97 (m, 2H), 0.72-0.67 (m, 2H). |
| tert-butyl 3-(2-(((2-bromopyridin-4- | |
| yl)amino)methyl)-6-cyclopropylimidazo[1,2- | |
| a]pyridin-8-yl)-3-hydroxyazetidine-1-carboxylate | |

### Synthesis of tert-butyl 4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazine-1-carboxylate

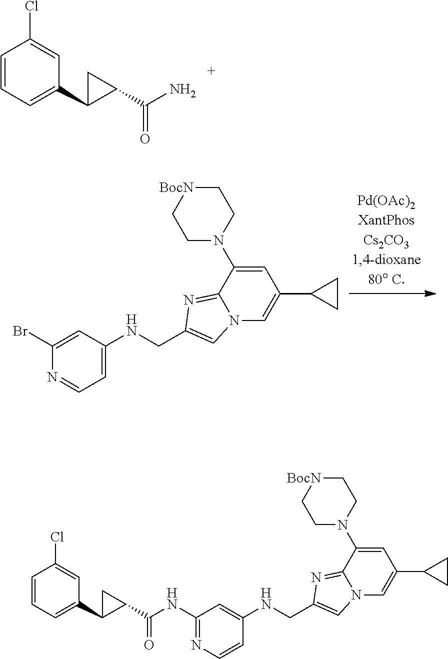 [SMILES: NC(=O)[C@H]1C[C@@H]1c1cccc(Cl)c1 ; CC(C)(C)OC(=O)N1CCN(c2cc(C3CC3)cn3cc(CNc4ccnc(Br)c4)nc23)CC1 ; CC(C)(C)OC(=O)N1CCN(c2cc(C3CC3)cn3cc(CNc4ccnc(NC(=O)[C@H]5C[C@@H]5c5cccc(Cl)c5)c4)nc23)CC1]

A mixture of tert-butyl 4-(2-(((2-bromopyridin-4-yl)amino)methyl)-6-cyclopropylimidazo[1,2-a]pyridin-8-yl)piperazine-1-carboxylate (0.085 g, 0.16 mmol), (1S,2S)-2-(3-chlorophenyl)cyclopropane-1-carboxamide (0.035 g, 0.18 mmol), Pd(OAc)₂ (0.007 g, 0.032 mmol), XantPhos (0.037 g, 0.065 mmol), and Cs₂CO₃ (0.11 g, 0.32 mmol) in 1,4-dioxane (5.0 mL) was degassed with nitrogen then stirred at 80° C. for 3 h. The mixture was filtered through Celite® and concentrated in vacuo. The residue was purified by column chromatography on silica gel, eluting with 0-20% (7 N NH₃ in MeOH) in DCM to give the title compound (0.071 g, 69%). ¹H NMR (400 MHz, CDCl₃) δ 7.83 (d, J=5.9 Hz, 1H), 7.56 (s, 1H), 7.52 (s, 1H), 7.35 (s, 1H), 7.24-7.17 (m, 2H), 7.09 (s, 1H), 7.03-6.99 (m, 1H), 6.29 (dd, J=2.3, 5.9 Hz, 1H), 6.22 (d, J=1.3 Hz, 1H), 5.04 (s, 1H), 4.49 (d, J=5.1 Hz, 2H), 3.70 (dd, J=5.0, 5.0 Hz, 4H), 3.44-3.43 (m, 4H), 2.60-2.54 (m, 1H), 1.86-1.65 (m, 3H), 1.50 (s, 9H), 1.38-1.32 (m, 1H), 0.95-0.89 (m, 2H), 0.66-0.62 (m, 2H).

Using a similar procedure to that used for tert-butyl 4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazine-1-carboxylate, the compounds in

Table 21 were prepared using (1S,2S)-2-(3-chlorophenyl)cyclopropane-1-carboxamide and an appropriate coupling partner, then purified by preparative LCMS.

TABLE 21

| | Coupling Partner/ |
|---|---|
| Compound | Analytical Data |
| 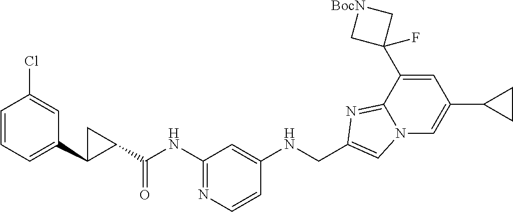 [SMILES: CC(C)(C)OC(=O)N1CC(F)(c2cc(C3CC3)cn3cc(CNc4ccnc(NC(=O)[C@H]5C[C@@H]5c5cccc(Cl)c5)c4)nc23)C1] | tert-butyl 3-(2-(((2-bromopyridin-4- yl)amino)methyl)-6- cyclopropylimidazo[1,2-a]pyridin-8-yl)-3- fluoroazetidine-1-carboxylate ¹H NMR (400 MHz, CDCl₃) δ 7.85-7.82 (m, 2H), 7.54-7.52 (m, 1H), 7.45 (s, 1H), 7.25-7.17 (m, 2H), 7.10 (s, 1H), 7.03-6.99 (m, 2H), 6.44 (dd, J = 1.9, 5.7 Hz, 1H), 5.38- 5.34 (m, 1H), 4.87 (s, 2H), 4.47 (s, 2H), 4.39-4.29 (m, 2H), 2.60-2.55 (m, 1H), 1.93-1.86 (m, 1H), 1.75-1.69 (m, 2H), 1.46 (s, 9H), 1.39-1.33 (m, 1H), 1.00-0.95 (m, 2H), 0.70-0.65 (m, 2H). |
| tert-butyl 3-(2-(((2-((1S,2S)-2-(3- | |
| chlorophenyl)cyclopropane-1-carboxamido)pyridin- | |
| 4-yl)amino)methyl)-6-cyclopropylimidazo[1,2- | |
| a]pyridin-8-yl)-3-fluoroazetidine-1-carboxylate | |
| 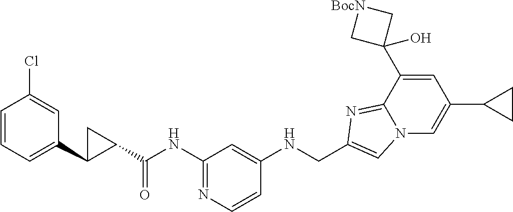 [SMILES: CC(C)(C)OC(=O)N1CC(O)(c2cc(C3CC3)cn3cc(CNc4ccnc(NC(=O)[C@H]5C[C@@H]5c5cccc(Cl)c5)c4)nc23)C1] tert-butyl 3-(2-(((2-((1S,2S)-2-(3- chlorophenyl)cyclopropane-1-carboxamido)pyridin- 4-yl)amino)methyl)-6-cyclopropylimidazo[1,2- a]pyridin-8-yl)-3-hydroxyazetidine-1-carboxylate | tert-butyl 3-(2-(((2-bromopyridin-4- yl)amino)methyl)-6- cyclopropylimidazo[1,2-a]pyridin-8-yl)-3- hydroxyazetidine-1-carboxylate ¹H NMR (400 MHz, CDCl₃) δ 7.84 (d, J = 5.9 Hz, 1H), 7.78 (s, 1H), 7.58 (s, 1H), 7.44 (s, 1H), 7.24-7.17 (m, 2H), 7.09 (dd, J = 2.0, 2.0 Hz, 1H), 7.05-6.99 (m, 2H), 6.29 (dd, J = 2.2, 5.8 Hz, 1H), 4.90-4.90 (m, 1H), 4.50 (d, J = 5.6 Hz, 2H), 4.30 (d, J = 9.0 Hz, 4H), 2.60-2.54 (m, 1H), 1.95-1.88 (m, 1H), 1.77-1.66 (m, 2H), 1.48 (s, 9H), 1.39-1.33 (m, 1H), 1.02-0.96 (m, 2H), 0.71-0.66 (m, 2H). |

### Example 132

### Synthesis of (1S,2S)-2-(3-chlorophenyl)-N-(4-(((6-cyclopropyl-8-(piperazin-1-yl)imidazo[1,2-a]pyridin-2-yl)methyl)amino)pyridin-2-yl)cyclopropane-1-carboxamide (I-132)

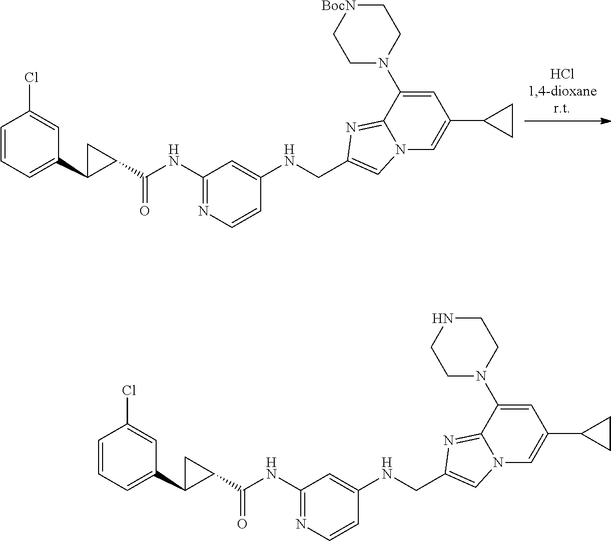 [SMILES: CC(C)(C)OC(=O)N1CCN(c2cc(C3CC3)cn3cc(CNc4ccnc(NC(=O)[C@H]5C[C@@H]5c5cccc(Cl)c5)c4)nc23)CC1 ; O=C(Nc1cc(NCc2cn3cc(C4CC4)cc(N4CCNCC4)c3n2)ccn1)[C@H]1C[C@@H]1c1cccc(Cl)c1]

(1S,2S)-2-(3-chlorophenyl)-N-(4-(((6-cyclopropyl-8-(piperazin-1-yl)imidazo[1,2-a]pyridin-2-yl)methyl)amino)pyridin-2-yl)cyclopropane-1-carboxamide (0.071 g, 0.11 mmol) was stirred in HCl solution (4.0 M in 1,4-dioxane, 0.28 mL, 1.1 mmol) in 1,4-dioxane (5.0 mL) at room temperature for 18 h. The mixture was diluted with methanol and loaded onto an SCX cartridge and washed with a mixture of methanol and DCM (1:4). The product was eluted with a mixture of 7 N NH₃ in MeOH in DCM (1:4) and concentrated in vacuo. The residue was purified by preparative HPLC to give the title compound (0.036 g, 60%). ESI-MS (M+H)+: 542.4, ¹H NMR (400 MHz, DMSO) δ 10.34 (s, 1H), 7.91 (s, 1H), 7.80 (d, J=5.8 Hz, 1H), 7.57 (s, 1H), 7.51-7.47 (m, 1H), 7.38-7.32 (m, 1H), 7.31-7.28 (m, 2H), 7.18 (d, J=7.3 Hz, 1H), 7.12 (dd, J=5.2, 5.2 Hz, 1H), 6.37 (dd, J=2.1, 5.7 Hz, 1H), 6.19 (s, 1H), 4.38 (d, J=5.6 Hz, 2H), 3.48-3.42 (m, 4H), 2.97-2.96 (m, 4H), 2.41-2.37 (m, 2H), 1.93-1.85 (m, 1H), 1.52-1.45 (m, 1H), 1.42-1.36 (m, 1H), 0.94-0.87 (m, 2H), 0.72-0.67 (m, 2H).

Using a similar procedure to that used for (1S,2S)-2-(3-chlorophenyl)-N-(4-(((6-cyclopropyl-8-(piperazin-1-yl)imidazo[1,2-a]pyridin-2-yl)methyl)amino)pyridin-2-yl)cyclopropane-1-carboxamide, the compounds in Table 22 were prepared and purified by preparative LCMS.

TABLE 22

| | | Prepared From/ |
|---|---|---|
| Example | Compound | Analytical Data |
| Example 133 | 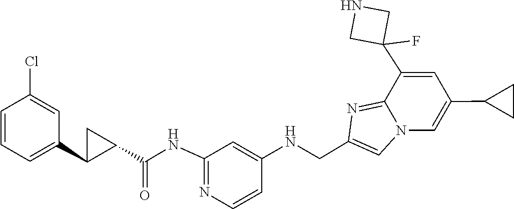 [SMILES: O=C(Nc1cc(NCc2cn3cc(C4CC4)cc(C4(F)CNC4)c3n2)ccn1)[C@H]1C[C@@H]1c1cccc(Cl)c1] (1S,2S)-2-(3-chlorophenyl)-N-(4-(((6- cyclopropyl-8-(3-fluoroazetidin-3- yl)imidazo[1,2-a]pyridin-2- yl)methyl)amino)pyridin-2- yl)cyclopropane-1-carboxamide, formic acid salt (I-133) | tert-butyl 3-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)-3-fluoroazetidine-1-carboxylate ESI-MS (M + H)+: 531.3, ¹H NMR (400 MHz, DMSO) δ 10.33 (s, 1H), 8.37 (s, 1H), 7.79-7.76 (m, 1H), 7.71 (s, 1H), 7.47 (s, 1H), 7.33 (dd, J = 7.8, 7.8 Hz, 1H), 7.26 (d, J = 7.7 Hz, 2H), 7.17-7.13 (m, 2H), 7.09 (s, 1H), 6.35 (dd, J = 2.1, 5.8 Hz, 1H), 4.44-4.34 (m, 4H), 3.97 (dd, J = 11.4, 21.5 Hz, 2H), 2.41-2.33 (m, 3H), 2.01-1.93 (m, 1H), 1.48-1.33 (m, 2H), 0.96- 0.90 (m, 2H), 0.73-0.69 (m, 2H). |
| Example 134 | 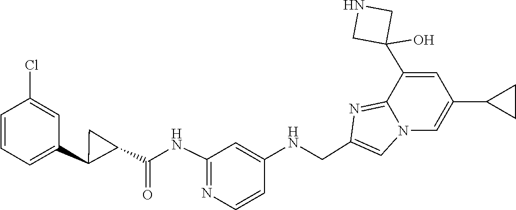 [SMILES: O=C(Nc1cc(NCc2cn3cc(C4CC4)cc(C4(O)CNC4)c3n2)ccn1)[C@H]1C[C@@H]1c1cccc(Cl)c1] (1S,2S)-2-(3-chlorophenyl)-N-(4-(((6- cyclopropyl-8-(3-hydroxyazetidin-3- yl)imidazo[1,2-a]pyridin-2- yl)methyl)amino)pyridin-2- yl)cyclopropane-1-carboxamide (I-134) | tert-butyl 3-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)-3-hydroxyazetidine-1-carboxylate ESI-MS (M + H)+: 529.3, ¹H NMR (400 MHz, MeOD) δ 8.16 (s, 1H), 7.79 (d, J = 6.0 Hz, 1H), 7.71 (s, 1H), 7.47 (s, 1H), 7.29 (dd, J = 7.8, 7.8 Hz, 1H), 7.23-7.19 (m, 3H), 7.13 (d, J = 7.7 Hz, 1H), 6.44 (dd, J = 2.3, 5.9 Hz, 1H), 4.56 (s, 2H), 4.41 (d, J = 10.8 Hz, 2H), 4.07 (d, J = 10.7 Hz, 2H), 2.51- 2.45 (m, 1H), 2.23-2.17 (m, 1H), 2.02- 1.92 (m, 1H), 1.65-1.59 (m, 1H), 1.39 (ddd, J = 4.5, 6.4, 8.3 Hz, 1H), 1.03-0.97 (m, 2H), 0.78-0.73 (m, 2H). |

### Example 135

### Synthesis of (1S,2S)—N-(4-(((8-(-azetidin-3-yl(hydroxy)methyl)-6-cyclopropylimidazo[1,2-a]pyridin-2-yl)methyl)amino)pyridin-2-yl)-2-(3-chlorophenyl)cyclopropane-1-carboxamide (I-135): Synthesis of tert-butyl 3-((2-(((2-bromopyridin-4-yl)amino)methyl)-6-cyclopropylimidazo[1,2-a]pyridin-8-yl)(hydroxy)methyl)azetidine-1-carboxylate

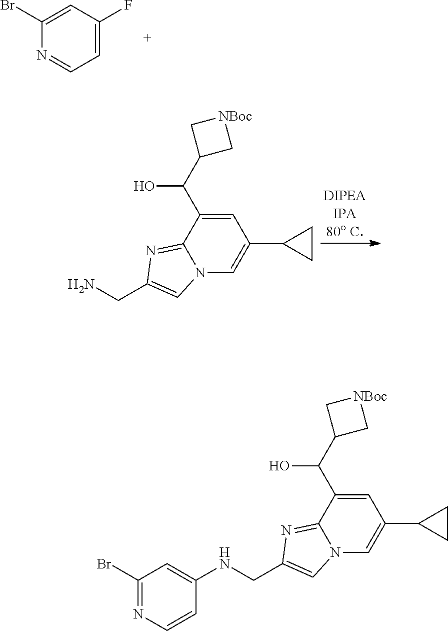 [SMILES: Fc1ccnc(Br)c1 ; CC(C)(C)OC(=O)N1CC(C(O)c2cc(C3CC3)cn3cc(CN)nc23)C1 ; CC(C)(C)OC(=O)N1CC(C(O)c2cc(C3CC3)cn3cc(CNc4ccnc(Br)c4)nc23)C1]

A mixture of tert-butyl 3-((2-(aminomethyl)-6-cyclopropylimidazo[1,2-a]pyridin-8-yl)(hydroxy)methyl)azetidine-1-carboxylate (0.082 g, 0.22 mmol), 2-bromo-4-fluoropyridine (0.039 g, 0.22 mmol), and DIPEA (0.076 mL, 0.44 mmol) in iPrOH (3.0 mL) was stirred at 80° C. for 48 h. The mixture was concentrated in vacuo and the residue was purified by column chromatography on silica gel, eluting with 0-20% methanol in DCM to give the title compound (0.060 g, 52%). ¹H NMR (400 MHz, CDCl₃) δ 7.94 (d, J=5.8 Hz, 1H), 7.78 (s, 1H), 7.39 (s, 1H), 6.81 (d, J=1.4 Hz, 1H), 6.73 (d, J=2.1 Hz, 1H), 6.48 (dd, J=2.1, 5.8 Hz, 1H), 4.46 (d, J=5.4 Hz, 2H), 4.06 (d, J=5.7 Hz, 1H), 3.90-3.84 (m, 2H), 3.76-3.71 (m, 2H), 3.18-3.12 (m, 1H), 1.90-1.85 (m, 1H), 1.45 (s, 9H), 1.01-0.95 (m, 2H), 0.68-0.63 (m, 2H).

### Synthesis of tert-butyl 3-((2-(((2-((1S,2S)-2-(3-chlorophenyl)cyclopropane-1-carboxamido)pyridin-4-yl)amino)methyl)-6-cyclopropylimidazo[1,2-a]pyridin-8-yl)(hydroxy)methyl)azetidine-1-carboxylate

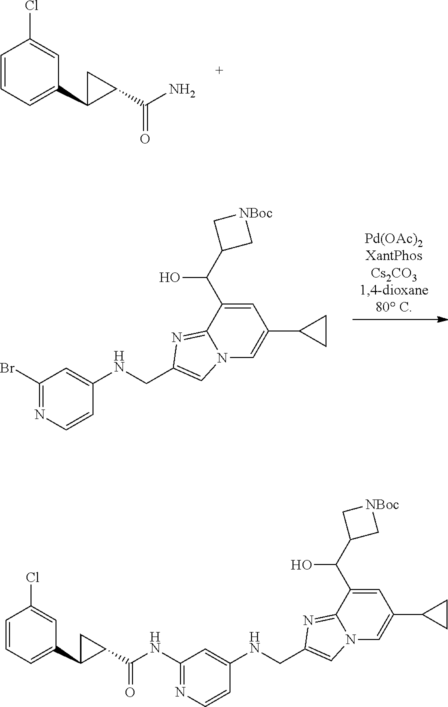 [SMILES: NC(=O)[C@H]1C[C@@H]1c1cccc(Cl)c1 ; CC(C)(C)OC(=O)N1CC(C(O)c2cc(C3CC3)cn3cc(CNc4ccnc(Br)c4)nc23)C1 ; "Pd(OAc)2XantPhosCs2CO31,4-dioxane80° C." ; CC(C)(C)OC(=O)N1CC(C(O)c2cc(C3CC3)cn3cc(CNc4ccnc(NC(=O)[C@H]5C[C@@H]5c5cccc(Cl)c5)c4)nc23)C1]

A mixture of tert-butyl 3-((2-(((2-bromopyridin-4-yl)amino)methyl)-6-cyclopropylimidazo[1,2-a]pyridin-8-yl)(hydroxy)methyl)azetidine-1-carboxylate (0.060 g, 0.11 mmol), (1S,2S)-2-(3-chlorophenyl)cyclopropane-1-carboxamide (0.024 g, 0.13 mmol), XantPhos (0.013 g, 0.023 mmol), and Cs₂CO₃ (0.074 g, 0.23 mmol) in 1,4-dioxane (5.0 mL) was degassed with nitrogen and Pd(OAc)₂ (0.003 g, 0.011 mmol) was added. The mixture was stirred at 80° C. for 3 h. The mixture was filtered through Celite® and concentrated in vacuo. The residue was purified by column chromatography on silica gel, eluting with 0-20% (7 N NH₃ in MeOH) in DCM to give the title compound (0.025 g, 34%). ¹H NMR (400 MHz, CDCl₃) δ 7.85 (d, J=5.6 Hz, 1H), 7.77 (s, 1H), 7.59 (s, 1H), 7.43 (s, 1H), 7.23-7.19 (m, 2H), 7.10 (s, 1H), 7.03-6.99 (m, 1H), 6.79 (d, J=1.1 Hz, 1H), 6.30 (dd, J=2.3, 5.8 Hz, 1H), 4.50 (d, J=5.4 Hz, 2H), 4.06 (d, J=6.4 Hz, 2H), 3.86-3.84 (m, 1H), 3.75-3.71 (m, 1H), 3.18-3.18 (m, 1H), 2.61-2.55 (m, 1H), 1.87 (dd, J=8.3, 8.3 Hz, 1H), 1.74-1.69 (m, 2H), 1.39-1.34 (m, 1H), 1.00-0.94 (m, 2H), 0.67-0.63 (m, 2H), −0.03 (s, 1H).

### Synthesis of (1S,2S)—N-(4-(((8-(-azetidin-3-yl(hydroxy)methyl)-6-cyclopropylimidazo[1,2-a]pyridin-2-yl)methyl)amino)pyridin-2-yl)-2-(3-chlorophenyl)cyclopropane-1-carboxamide (I-135)

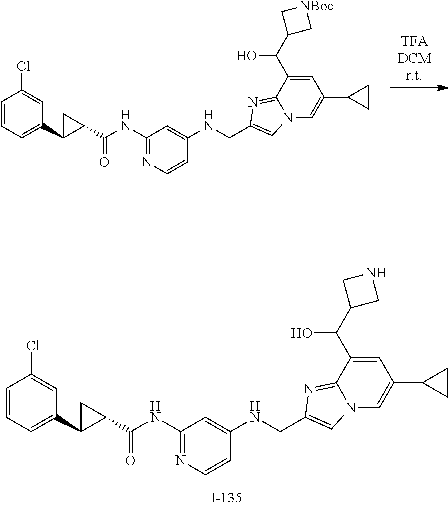 [SMILES: CC(C)(C)OC(=O)N1CC(C(O)c2cc(C3CC3)cn3cc(CNc4ccnc(NC(=O)[C@H]5C[C@@H]5c5cccc(Cl)c5)c4)nc23)C1 ; "TFADCMr.t." ; O=C(Nc1cc(NCc2cn3cc(C4CC4)cc(C(O)C4CNC4)c3n2)ccn1)[C@H]1C[C@@H]1c1cccc(Cl)c1]

tert-Butyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)(hydroxy)methyl)azetidine-1-carboxylate (0.025 g, 0.039 mmol) was stirred with trifluoroacetic acid (0.10 mL, 1.3 mmol) in DCM (2.0 mL) at room temperature for 1 h. The mixture was concentrated in vacuo and the residue was loaded onto an SCX cartridge and washed with a mixture of methanol and DCM. The product was eluted with a mixture of 7 N NH₃ in MeOH in DCM and concentrated in vacuo. The residue was purified by preparative HPLC to give the title compound (0.016 g, 76%) as a trifluoroacetic acid salt. ESI-MS (M+H)+: 543.4¹H NMR (400 MHz, MeOD) δ 8.24 (s, 1H), 7.93-7.81 (m, 2H), 7.46 (s, 1H), 7.34-7.23 (m, 3H), 7.15 (d, J=7.7 Hz, 1H), 6.79 (s, 1H), 6.38 (s, 1H), 5.35 (d, J=3.1 Hz, 1H), 4.71 (s, 2H), 4.23 (dd, J=3.6, 11.2 Hz, 2H), 4.15 (dd, J=6.6, 10.6 Hz, 1H), 3.89 (dd, J=9.7, 9.7 Hz, 1H), 3.52-3.42 (m, 1H), 2.68-2.62 (m, 1H), 2.13-2.00 (m, 2H), 1.80-1.74 (m, 1H), 1.58 (ddd, J=4.6, 6.7, 8.2 Hz, 1H), 1.10-1.04 (m, 2H), 0.78 (q, J=5.0 Hz, 2H).

### Example 137

### Synthesis of (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide (I-137)

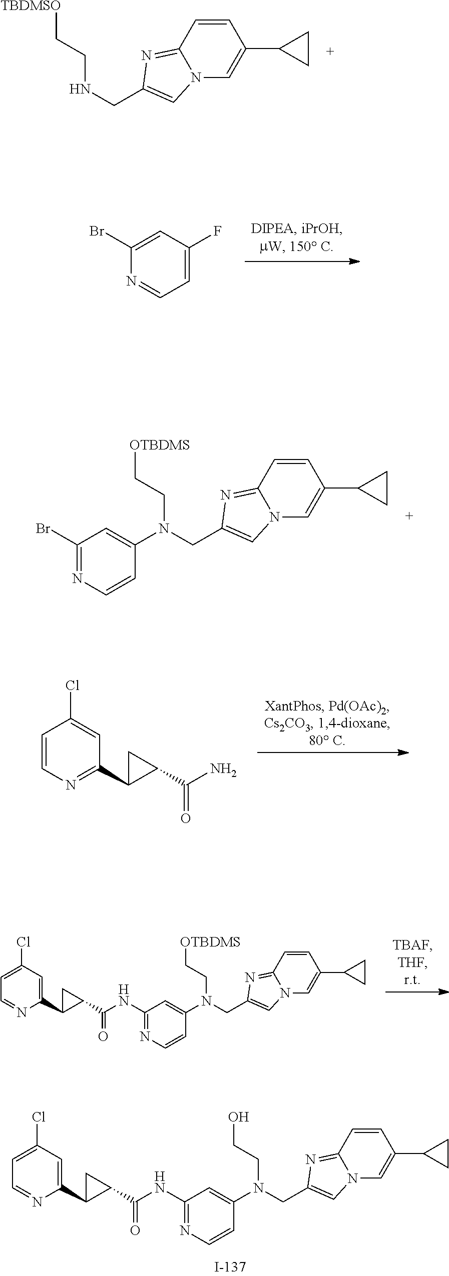

Synthesis of 2-bromo-N-(2-((tert-butyldimethylsilyl)oxy)ethyl)-N-((6-cyclopropylimidazo[1,2-a]pyridin-2-yl)methyl)pyridin-4-amine. 2-bromo-4-fluoropyridine (150 mg, 0.85 mmol), 2-((tert-butyldimethylsilyl)oxy)-N-((6-cyclopropylimidazo[1,2-a]pyridin-2-yl)methyl)ethan-1-amine (290 mg, 0.85 mmol), and DIPEA (0.44 mL, 2.6 mmol) in iPrOH (4.0 mL) were heated to 150° C. in a microwave for 12 h. The mixture was cooled to room temperature and concentrated in vacuo. The residue was purified by column chromatography on silica gel, eluting with a gradient of 0-10% (7 N NH₃ in MeOH) in DCM to give the title compound (100 mg, 23%). ESI-MS (M+H)+: 501, 503, ¹H NMR (400 MHz, CDCl₃) δ 7.86 (d, J=6.1 Hz, 1H), 7.76 (d, J=0.5 Hz, 1H), 7.41 (d, J=9.3 Hz, 1H), 7.19 (s, 1H), 6.91 (dd, J=1.7, 9.3 Hz, 1H), 6.78 (d, J=2.4 Hz, 1H), 6.53 (dd, J=2.4, 6.1 Hz, 1H), 4.69 (s, 2H), 3.82 (dd, J=5.6, 5.6 Hz, 2H), 3.60 (dd, J=5.6, 5.6 Hz, 2H), 1.88-1.81 (m, 1H), 0.95-0.88 (m, 2H), 0.84 (s, 9H), 0.65-0.60 (m, 2H), 0.00 (s, 6H).

Synthesis of (1S,2S)—N-(4-((2-((tert-butyldimethylsilyl)oxy)ethyl)((6-cyclopropylimidazo[1,2-a]pyridin-2-yl)methyl)amino)pyridin-2-yl)-2-(4-chloropyridin-2-yl)cyclopropane-1-carboxamide. (1S,2S)-2-(4-chloropyridin-2-yl)cyclopropane-1-carboxamide (86 mg, 0.44 mmol), Pd(OAc)₂ (9.0 mg, 0.04 mmol), XantPhos (46 mg, 0.08 mmol), and Cs₂CO₃ (260 mg, 0.80 mmol) were placed under N₂ atmosphere. A solution of 2-bromo-N-(2-((tert-butyldimethylsilyl)oxy)ethyl)-N-((6-cyclopropylimidazo[1,2-a]pyridin-2-yl)methyl)pyridin-4-amine (200 mg, 0.40 mmol) in 1,4-dioxane (0.5 mL) was added, and the reaction mixture was degassed for 10 min and stirred at 80° C. for 16 h. The reaction mixture was allowed to cool to room temperature, filtered through Celite®, washed with MeOH, and concentrated in vacuo. The residue was purified by column chromatography on silica gel, eluting with a gradient of 0-10% (7N NH₃ in MeOH) in DCM to give the title compound (90 mg, 37%). ESI-MS (M+H)+: 617.4, ¹H NMR (400 MHz, CDCl₃) δ 9.88 (s, 1H), 8.18 (d, J=5.6 Hz, 1H), 7.77 (s, 1H), 7.73 (s, 1H), 7.62 (d, J=6.3 Hz, 1H), 7.42 (d, J=9.3 Hz, 1H), 7.33 (s, 1H), 7.22 (d, J=1.5 Hz, 1H), 7.02 (dd, J=2.0, 5.3 Hz, 1H), 6.89 (dd, J=1.6, 9.2 Hz, 1H), 6.22 (dd, J=2.5, 6.3 Hz, 1H), 4.75 (s, 2H), 3.86 (dd, J=5.6, 5.6 Hz, 2H), 3.71-3.65 (m, 2H), 2.61-2.55 (m, 1H), 1.88-1.80 (m, 1H), 1.72-1.66 (m, 1H), 0.95-0.90 (m, 2H), 0.85 (s, 9H), 0.65-0.59 (m, 2H), 0.00 (s, 6H).

Synthesis of (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide (I-137). (1S,2S)—N-(4-((2-((tert-butyldimethylsilyl)oxy)ethyl)((6-cyclopropylimidazo[1,2-a]pyridin-2-yl)methyl)amino)pyridin-2-yl)-2-(4-chloropyridin-2-yl)cyclopropane-1-carboxamide (90 mg, 0.15 mmol) was stirred with a solution of TBAF (1M in THF, 0.29 mL, 0.29 mmol) in DCM (1.0 mL) at room temperature for 4 h. The reaction mixture was concentrated in vacuo. The residue was purified by column chromatography on silica gel, eluting with a gradient of 0-10% (7 N NH₃ in MeOH) in DCM and freeze-dried to give the title compound (60 mg, 82%). ESI-MS (M+H)+: 503.0, ¹H NMR (400 MHz, DMSO) δ 10.40 (s, 1H), 8.42 (d, J=5.3 Hz, 1H), 8.29 (s, 1H), 7.79 (d, J=5.8 Hz, 1H), 7.65-7.62 (m, 2H), 7.58 (s, 1H), 7.38 (d, J=9.3 Hz, 1H), 7.33 (dd, J=2.1, 5.4 Hz, 1H), 6.98 (dd, J=1.8, 9.3 Hz, 1H), 6.45 (dd, J=2.4, 6.2 Hz, 1H), 5.35 (s, 1H), 4.66 (s, 2H), 3.66 (dd, J=5.7, 5.7 Hz, 2H), 3.57 (dd, J=6.4, 6.4 Hz, 2H), 2.63-2.52 (m, 2H), 1.95-1.87 (m, 1H), 1.53-1.44 (m, 2H), 0.93-0.87 (m, 2H), 0.69-0.64 (m, 2H).

### Synthesis of 2-bromo-N-(1-(6-cyclopropylimidazo[1,2-a]pyridin-2-yl)-2,2,2-trifluoroethyl)pyridin-4-amine

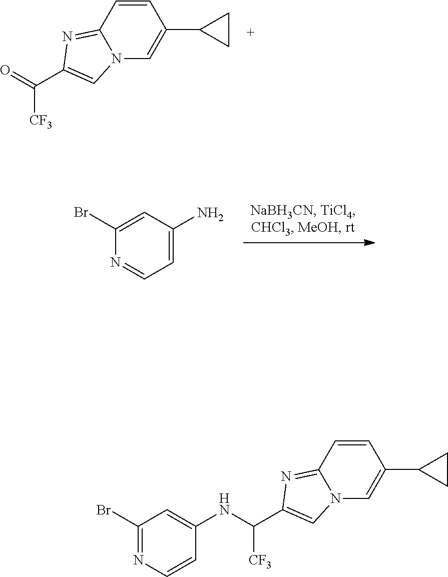 [SMILES: O=C(c1cn2cc(C3CC3)ccc2n1)C(F)(F)F ; Nc1ccnc(Br)c1 ; "NaBH3CN, TiCl4,CHCl3, MeOH, rt" ; FC(F)(F)C(Nc1ccnc(Br)c1)c1cn2cc(C3CC3)ccc2n1]

TiCl₄ (0.10 mL, 0.87 mmol) was added dropwise to a solution of 1-(6-cyclopropylimidazo[1,2-a]pyridin-2-yl)-2,2,2-trifluoroethan-1-one (220 mg, 0.87 mmol) and 2-bromopyridin-4-amine (300 mg, 1.7 mmol) in CHCl₃ (10 mL), and the reaction mixture was stirred at room temperature for 16 h. A solution of NaCNBH₃ (110 mg, 1.7 mmol) in MeOH (3.0 mL) was added dropwise and the reaction mixture was stirred at room temperature for 5 h. The reaction mixture was quenched with NaHCO₃ (sat·aq·, 20 mL) and extracted with DCM (3×40 mL). The combined organics were dried over MgSO₄ and concentrated in vacuo. The residue was purified by column chromatography on silica gel, eluting with a gradient of 30-40% EtOAc in cyclohexane to give the title compound (80 mg, 22%) at ˜50% purity. ESI-MS (M+H)+: 411, 413.

### Synthesis of tert-butyl 6-(2-(aminomethyl)-6-cyclopropylimidazo[1,2-a]pyridin-8-yl)-2,6-diazaspiro[3.3]heptane-2-carboxylate

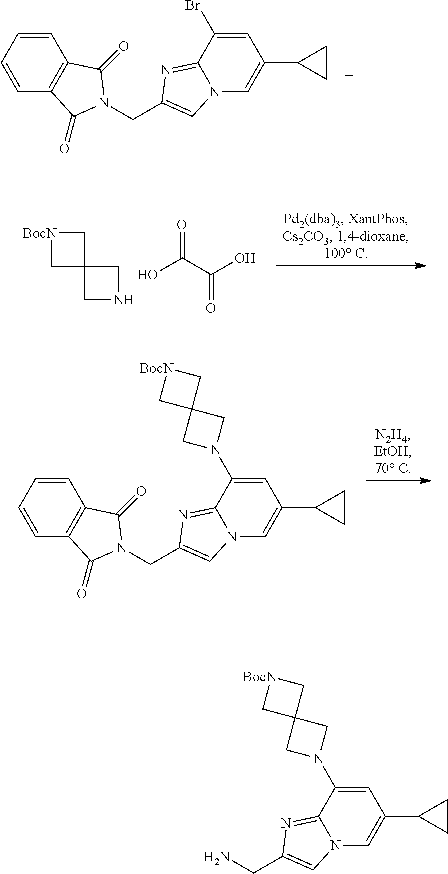

Synthesis of tert-butyl 6-(6-cyclopropyl-2-((1,3-dioxoisoindolin-2-yl)methyl)imidazo[1,2-a]pyridin-8-yl)-2,6-diazaspiro[3.3]heptane-2-carboxylate. A suspension of 2-((8-bromo-6-cyclopropylimidazo[1,2-a]pyridin-2-yl)methyl)isoindoline-1,3-dione (300 mg, 0.76 mmol), tert-butyl 2,6-diazaspiro[3.3]heptane-2-carboxylate oxalate (330 mg, 1.1 mmol), XantPhos (88 mg, 0.15 mmol), and Cs₂CO₃ (740 mg, 2.3 mmol) in 1,4-dioxane (6.0 mL) was degassed for 10 min. Pd₂(dba)₃ (69 mg, 0.08 mmol) was added and the reaction mixture was stirred at 100° C. for 3 h. The reaction mixture was allowed to cool to room temperature, filtered through Celite®, and concentrated in vacuo. The residue was purified by column chromatography on silica gel, eluting with a gradient of 0-10% EtOAc in cyclohexane to give the title compound (190 mg, 49%). ESI-MS (M+H)+: 514.2

Synthesis of tert-butyl 6-(2-(aminomethyl)-6-cyclopropylimidazo[1,2-a]pyridin-8-yl)-2,6-diazaspiro[3.3]heptane-2-carboxylate. Using a similar procedure to that for 3-(2-(aminomethyl)-6-cyclopropylimidazo[1,2-a]pyridin-8-yl)oxetan-3-ol, the title compound was synthesized from tert-butyl 6-(6-cyclopropyl-2-((1,3-dioxoisoindolin-2-yl)methyl)imidazo[1,2-a]pyridin-8-yl)-2,6-diazaspiro[3.3]heptane-2-carboxylate using hydrazine hydrate and used without further purification. ESI-MS (M+H)+: 384.3, ¹H NMR (400 MHz, CDCl₃) δ 7.36 (s, 1H), 7.28 (s, 1H), 5.67 (d, J=1.4 Hz, 1H), 4.29 (s, 4H), 4.11 (s, 4H), 3.94 (s, 2H), 1.84-1.78 (m, 1H), 1.45 (s, 9H), 0.92-0.86 (m, 2H), 0.65-0.60 (m, 2H).

### Synthesis of N-(2-(((2-bromopyridin-4-yl)amino)methyl)-6-cyclopropylimidazo[1,2-a]pyridin-8-yl)-N-methylacetamide

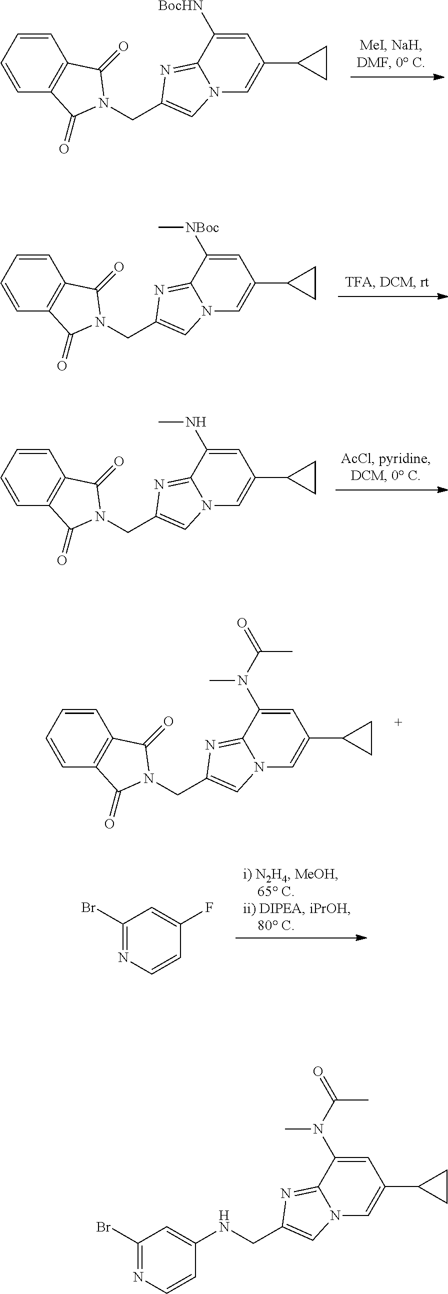

Synthesis of tert-butyl (6-cyclopropyl-2-((1,3-dioxoisoindolin-2-yl)methyl)imidazo[1,2-a]pyridin-8-yl)(methyl)carbamate. A solution of tert-butyl (6-cyclopropyl-2-((1,3-dioxoisoindolin-2-yl)methyl)imidazo[1,2-a]pyridin-8-yl)carbamate (500 mg, 1.2 mmol) in DMF (10 mL) was cooled to 0° C. NaH (60% in mineral oil, 56 mg, 1.4 mmol) was added and the reaction mixture was stirred at 0° C. for 30 min. MeI (86 μL, 1.4 mmol) was added and the reaction mixture was stirred at 0° C. for 2 h. The reaction mixture was allowed to warm to room temperature, quenched with water (10 mL), and extracted with EtOAc (3×20 mL). The combined organics were passed through a hydrophobic frit and concentrated in vacuo. The residue was purified by column chromatography on silica gel, eluting with a gradient of 0-100% EtOAc in cyclohexane to give the title compound (195 mg, 38%). ¹H NMR (400 MHz, CDCl₃) δ 7.87 (dd, J=3.0, 5.6 Hz, 2H), 7.72 (dd, J=3.0, 5.6 Hz, 2H), 7.68 (s, 1H), 7.41 (s, 1H), 6.81 (s, 1H), 5.05 (s, 2H), 3.34 (s, 3H), 1.88-1.80 (m, 1H), 1.37 (s, 9H), 0.96-0.89 (m, 2H), 0.65-0.59 (m, 2H).

Synthesis of 2-((6-cyclopropyl-8-(methylamino)imidazo[1,2-a]pyridin-2-yl)methyl)isoindoline-1,3-dione. Trifluoroacetic acid (0.14 mL, 1.8 mmol) was added to a solution of tert-butyl (6-cyclopropyl-2-((1,3-dioxoisoindolin-2-yl)methyl)imidazo[1,2-a]pyridin-8-yl)(methyl)carbamate (195 mg, 0.45 mmol) in DCM (10 mL) and the reaction mixture was stirred at room temperature for 5 h. The reaction mixture was quenched with NaHCO₃ (sat·aq·, 35 mL) and extracted with DCM (2×50 mL). The combined organics were passed through a hydrophobic frit and concentrated in vacuo to give the title compound (106 mg, 71%). ESI-MS (M+H)+: 347.2

Synthesis of N-(6-cyclopropyl-2-((1,3-dioxoisoindolin-2-yl)methyl)imidazo[1,2-a]pyridin-8-yl)-N-methylacetamide. Acetyl chloride (21 μL, 0.29 mmol) was added to a solution of 2-((6-cyclopropyl-8-(methylamino)imidazo[1,2-a]pyridin-2-yl)methyl)isoindoline-1,3-dione (100 mg, 0.29 mmol) and pyridine (0.07 mL, 0.87 mmol) in DCM (1.0 mL) at 0° C., and the reaction mixture was stirred at 0° C. for 3 h. The reaction mixture was allowed to warm to room temperature, quenched with water (5 mL), and extracted with DCM (3×15 mL). The combined organics were dried over a hydrophobic frit and concentrated in vacuo. The residue was purified by column chromatography on silica gel, eluting with a gradient of 0-100% EtOAc in cyclohexane to give the title compound (87 mg, 78%). ESI-MS (M+H)+: 384.2, ¹H NMR (400 MHz, CDCl₃) δ 7.88 (dd, J=3.0, 5.6 Hz, 2H), 7.79 (s, 1H), 7.74 (dd, J=3.0, 5.3 Hz, 2H), 7.47 (s, 1H), 6.78 (s, 1H), 5.06 (s, 2H), 3.33 (s, 3H), 1.88-1.81 (m, 1H), 1.26 (s, 3H), 1.26 (t, J=7.2 Hz, 3H), 0.97 (d, J=8.3 Hz, 2H), 0.66-0.60 (m, 2H).

Synthesis of N-(2-(((2-bromopyridin-4-yl)amino)methyl)-6-cyclopropylimidazo[1,2-a]pyridin-8-yl)-N-methylacetamide. Using a similar procedure to that for 3-(2-(aminomethyl)-6-cyclopropylimidazo[1,2-a]pyridin-8-yl)oxetan-3-ol, N-(2-(aminomethyl)-6-cyclopropylimidazo[1,2-a]pyridin-8-yl)-N-methylacetamide (44 mg, 0.17 mmol) was synthesized from N-(6-cyclopropyl-2-((1,3-dioxoisoindolin-2-yl)methyl)imidazo[1,2-a]pyridin-8-yl)-N-methylacetamide using hydrazine hydrate. Without further purification. this was dissolved in iPrOH (5.0 mL), then DIPEA (0.09 mL, 0.51 mmol) and 2-bromo-4-fluoropyridine (30 mg, 0.17 mmol) were added. The reaction mixture was stirred at 80° C. for 16 h. The reaction mixture was allowed to cool to room temperature, water (10 mL) was added, and the reaction mixture was extracted with EtOAc (3×20 mL). The combined organics were dried over a hydrophobic frit and concentrated in vacuo. The residue was purified by column chromatography on silica gel, eluting with a gradient of 0-100% EtOAc in cyclohexane followed by 0-20% (7 N NH₃ in MeOH) in EtOAc to give the title compound (45 mg, 63%). ESI-MS (M+H)+: 416.1, ¹H NMR (400 MHz, CDCl₃) δ 7.91 (d, J=5.8 Hz, 1H), 7.87 (s, 1H), 7.48 (s, 1H), 6.85 (s, 1H), 6.70 (d, J=2.3 Hz, 1H), 6.47 (dd, J=2.0, 5.8 Hz, 1H), 5.40 (dd, J=5.6, 5.6 Hz, 1H), 4.48 (d, J=5.6 Hz, 2H), 3.36 (s, 3H), 1.94 (br s, 4H), 1.05-0.97 (m, 2H), 0.72-0.64 (m, 2H).

### Synthesis of N-(2-(((2-bromopyridin-4-yl)amino)methyl)-6-cyclopropylimidazo[1,2-a]pyridin-8-yl)acetamide

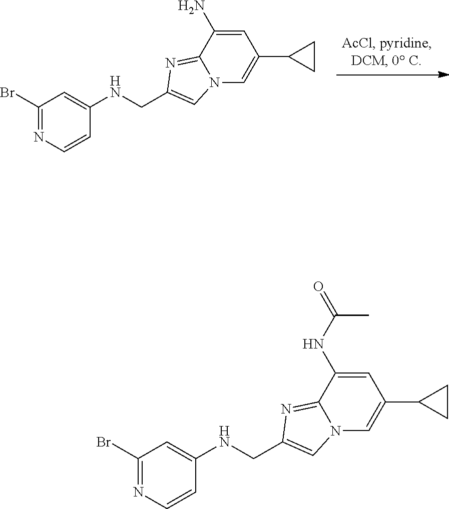 [SMILES: Nc1cc(C2CC2)cn2cc(CNc3ccnc(Br)c3)nc12 ; "AcCl, pyridine,DCM, 0° C." ; CC(=O)Nc1cc(C2CC2)cn2cc(CNc3ccnc(Br)c3)nc12]

Acetyl chloride (12 μL, 0.17 mmol) was added to a solution of 2-(((2-bromopyridin-4-yl)amino)methyl)-6-cyclopropylimidazo[1,2-a]pyridin-8-amine (60 mg, 0.17 mmol) and pyridine (0.04 mL, 0.48 mmol) in DCM (0.3 mL) at 0° C. and the reaction mixture was stirred at 0° C. for 2 h. The reaction mixture was allowed to warm to room temperature, quenched with water (3 mL), and extracted with EtOAc (3×10 mL). The combined organics were dried over a hydrophobic frit and concentrated in vacuo. The residue was purified by column chromatography on silica gel, eluting with a gradient of 0-10% (7N NH₃ in MeOH) in DCM to give the title compound (75 mg, quant). ESI-MS (M+H)+: 400.1, 402.1

### Synthesis of tert-Butyl 4-(2-(((2-bromopyridin-4-yl)amino)methyl)-6-cyclopropylimidazo[1,2-a]pyridin-8-yl)piperazine-1-carboxylate

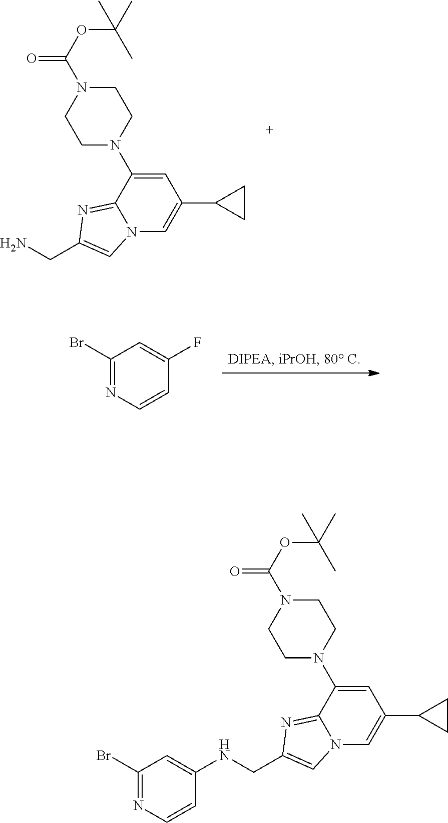 [SMILES: CC(C)(C)OC(=O)N1CCN(c2cc(C3CC3)cn3cc(CN)nc23)CC1 ; Fc1ccnc(Br)c1 ; "DIPEA, iPrOH, 80° C." ; CC(C)(C)OC(=O)N1CCN(c2cc(C3CC3)cn3cc(CNc4ccnc(Br)c4)nc23)CC1]

A solution of tert-butyl 4-(2-(aminomethyl)-6-cyclopropylimidazo[1,2-a]pyridin-8-yl)piperazine-1-carboxylate (300 mg, 0.80 mmol), 2-bromo-4-fluoropyridine (150 mg, 0.84 mmol), and DIPEA (0.28 mL, 1.6 mmol) in iPrOH (5.0 mL) was stirred at 80° C. for 16 h. The reaction mixture was allowed to cool to room temperature and concentrated in vacuo. The residue was purified by column chromatography on silica gel, eluting with a gradient of 0-100% EtOAc in cyclohexane to give the title compound (170 mg, 40%). ¹H NMR (400 MHz, CDCl₃): δ, ppm, 7.93 (d, J=5.9 Hz, 1H), 7.53 (d, J=1.4 Hz, 1H), 7.33 (s, 1H), 6.73 (d, J=2.2 Hz, 1H), 6.47 (dd, J=2.2, 5.9 Hz, 1H), 6.24 (d, J=1.4 Hz, 1H), 5.06 (t, J=5.3 Hz, 1H), 4.44 (d, J=5.3 Hz, 2H), 3.71 (t, J=5.0 Hz, 4H), 3.43 (t, J=5.0 Hz, 4H), 1.88-1.81 (m, 1H), 0.96-0.90 (m, 2H), 0.67-0.62 (m, 2H). ESI-MS (M+H)+: 527.1, 529.0.

Using a similar procedure to that used for tert-Butyl 4-(2-(((2-bromopyridin-4-yl)amino)methyl)-6-cyclopropylimidazo[1,2-a]pyridin-8-yl)piperazine-1-carboxylate, the compound in Table 23 was prepared from tert-butyl 6-(2-(aminomethyl)-6-cyclopropylimidazo[1,2-a]pyridin-8-yl)-2,6-diazaspiro[3.3]heptane-2-carboxylate and 2-bromo-4-fluoropyridine.

TABLE 23

| | Coupling Partner/ |
|---|---|
| Compound | Analytical Data |
| 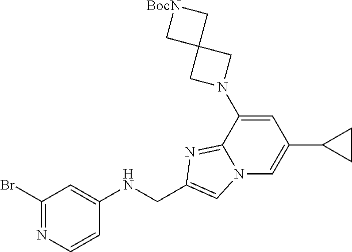 [SMILES: CC(C)(C)OC(=O)N1CC2(C1)CN(c1cc(C3CC3)cn3cc(CNc4ccnc(Br)c4)nc13)C2] tert-butyl 6-(2-(((2-bromopyridin-4- yl)amino)methyl)-6-cyclopropylimidazo[1,2- a]pyridin-8-yl)-2,6-diazaspiro[3.3]heptane-2- carboxylate | tert-butyl 6-(2-(aminomethyl)-6- cyclopropylimidazo[1,2-a]pyridin-8-yl)- 2,6-diazaspiro[3.3]heptane-2-carboxylate ESI-MS (M + H)+: 539.1, 541.0, ¹H NMR (400 MHz, CDCl₃) δ 7.92 (d, J = 5.8 Hz, 1H), 7.36 (s, 1H), 7.30 (s, 1H), 6.75 (d, J = 2.1 Hz, 1H), 6.47 (dd, J = 2.2, 5.8 Hz, 1H), 5.72 (s, 1H), 5.08 (s, 1H), 4.41 (d, J = 5.1 Hz, 2H), 4.31 (s, 4H), 4.13 (s, 4H), 1.85-1.77 (m, 1H), 1.46 (s, 9H), 0.93- 0.86 (m, 2H), 0.66-0.61 (m, 2H). |

### Synthesis of 2-bromo-N-((6-cyclopropyl-8-(4-methylpiperazin-1-yl)imidazo[1,2-a]pyridin-2-yl)methyl)pyridin-4-amine

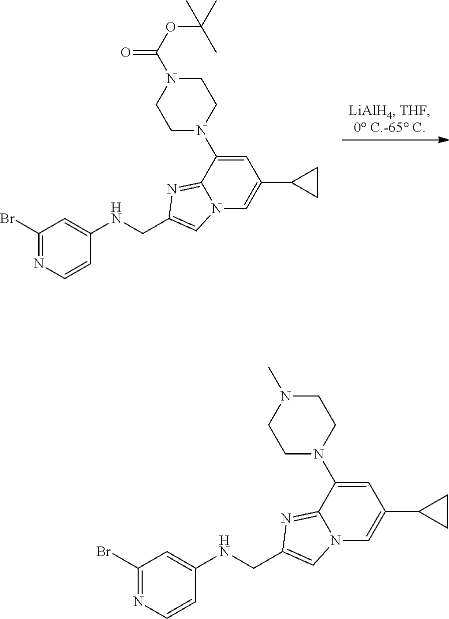 [SMILES: CC(C)(C)OC(=O)N1CCN(c2cc(C3CC3)cn3cc(CNc4ccnc(Br)c4)nc23)CC1 ; "LiAlH4, THF,0° C.-65° C." ; CN1CCN(c2cc(C3CC3)cn3cc(CNc4ccnc(Br)c4)nc23)CC1]

A solution of tert-butyl 4-(2-(((2-bromopyridin-4-yl)amino)methyl)-6-cyclopropylimidazo[1,2-a]pyridin-8-yl)piperazine-1-carboxylate (85 mg, 0.16 mmol) in THF (5.0 mL) was cooled to 0° C., and LiAlH₄ (1 M in THF, 0.48 mL, 0.48 mmol) was added dropwise. The reaction mixture was stirred at 65° C. for 3 h. The reaction mixture was allowed to cool to room temperature, quenched with NH₄Cl (sat·aq·, 10 mL), and extracted with DCM (3×20 mL). The combined organics were dried over MgSO₄ and concentrated in vacuo to give the title compound which was used without further purification. ESI-MS (M+H)+: 441.1, 443.1 ¹H NMR (400 MHz, CDCl₃): δ ppm, 8.20 (ddd, J=1.3, 1.3, 6.2 Hz, 1H), 7.50 (ddd, J=0.8, 1.3, 4.8 Hz, 1H), 7.32-7.30 (m, 1H), 6.53 (ddd, J=1.3, 1.3, 6.2 Hz, 1H), 6.23 (dd, J=1.3, 4.8 Hz, 1H), 4.45 (dd, J=5.2, 13.3 Hz, 2H), 3.53 (br s, 4H), 2.70 (t, J=4.7 Hz, 4H), 2.40 (s, 3H), 1.88-1.80 (m, 1H), 0.95-0.89 (m, 2H), 0.67-0.63 (m, 2H), aromatic CH obscured by CDCl₃ peak.

### Synthesis of 2-bromo-N-((6-cyclopropyl-8-(6-methyl-2,6-diazaspiro[3.3]heptan-2-yl)imidazo[1,2-a]pyridin-2-yl)methyl)pyridin-4-amine

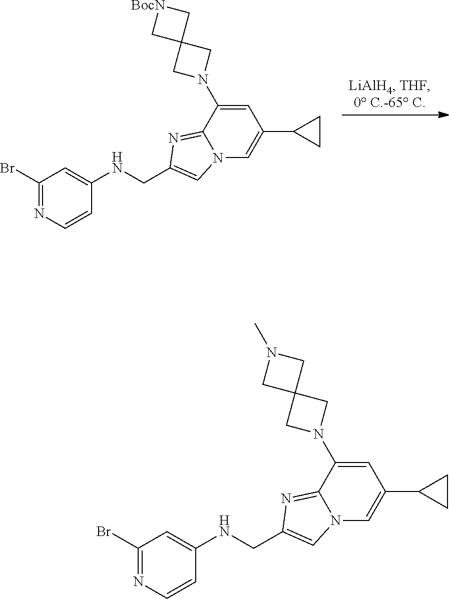 [SMILES: CC(C)(C)OC(=O)N1CC2(C1)CN(c1cc(C3CC3)cn3cc(CNc4ccnc(Br)c4)nc13)C2 ; "LiAlH4, THF,0° C.-65° C." ; CN1CC2(C1)CN(c1cc(C3CC3)cn3cc(CNc4ccnc(Br)c4)nc13)C2]

Using a similar procedure to that used for 2-bromo-N-((6-cyclopropyl-8-(4-methylpiperazin-1-yl)imidazo[1,2-a]pyridin-2-yl)methyl)pyridin-4-amine the title compound was prepared from tert-butyl 6-(2-(((2-bromopyridin-4-yl)amino)methyl)-6-cyclopropylimidazo[1,2-a]pyridin-8-yl)-2,6-diazaspiro[3.3]heptane-2-carboxylate and LiAlH₄. ESI-MS (M+H)+: 441.1, 443.1, ¹H NMR (400 MHz, CDCl₃) δ 7.92 (d, J=5.8 Hz, 1H), 7.34 (s, 1H), 7.28 (s, 1H), 6.76 (d, J=2.0 Hz, 1H), 6.47 (dd, J=2.3, 5.8 Hz, 1H), 5.70 (d, J=1.3 Hz, 1H), 5.11 (t, J=4.6 Hz, 1H), 4.39 (d, J=5.0 Hz, 2H), 4.28 (s, 4H), 3.52 (s, 4H), 2.39 (s, 3H), 1.83-1.76 (m, 1H), 0.93-0.86 (m, 2H), 0.65-0.60 (m, 2H).

### Example 138

### Synthesis of (1S,2S)-2-(3-chlorophenyl)-N-(4-(((6-cyclopropyl-8-(4-methylpiperazin-1-yl)imidazo[1,2-a]pyridin-2-yl)methyl)amino)pyridin-2-yl)cyclopropane-1-carboxamide (I-138)

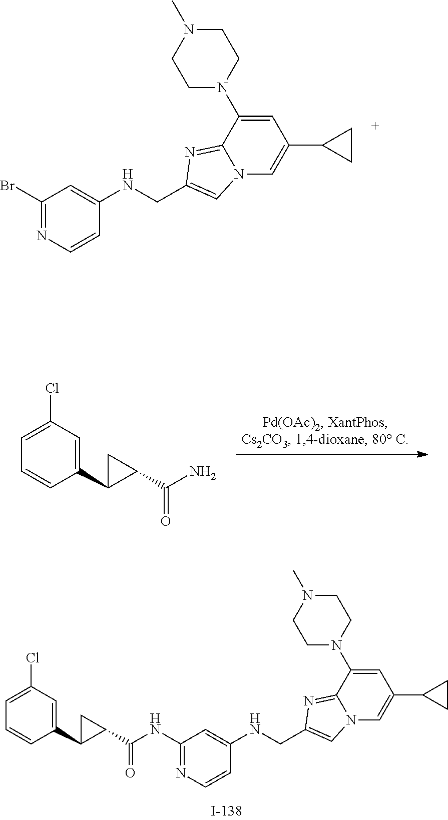 [SMILES: CN1CCN(c2cc(C3CC3)cn3cc(CNc4ccnc(Br)c4)nc23)CC1 ; NC(=O)[C@H]1C[C@@H]1c1cccc(Cl)c1 ; "Pd(OAc)2, XantPhos,Cs2CO3, 1,4-dioxane, 80° C." ; CN1CCN(c2cc(C3CC3)cn3cc(CNc4ccnc(NC(=O)[C@H]5C[C@@H]5c5cccc(Cl)c5)c4)nc23)CC1]

2-Bromo-N-((6-cyclopropyl-8-(4-methylpiperazin-1-yl)imidazo[1,2-a]pyridin-2-yl)methyl)pyridin-4-amine (69 mg, 0.16 mmol), (1S,2S)-2-(3-chlorophenyl)cyclopropane-1-carboxamide (31 mg, 0.16 mmol), XantPhos (18 mg, 0.03 mmol), Cs₂CO₃ (100 mg, 0.31 mmol), and Pd(OAc)₂ (3.5 mg, 0.015 mmol) were placed under a N₂ atmosphere. 1,4-Dioxane (1.5 mL) was added and the reaction mixture was stirred at 80° C. for 16 h. The reaction mixture was cooled to room temperature, filtered through Celite®, washed with EtOAc (30 mL), and concentrated in vacuo. The residue was purified by column chromatography on silica gel, eluting with a gradient of 0-20% 7 N NH₃ in MeOH in DCM. The residue was further purified by column chromatography on silica gel, eluting with 4% 7 N NH₃ in MeOH in DCM. The residue was further purified by preparative HPLC to give of the title compound (10 mg, 11%) as a formic acid salt. ESI-MS (M+H)+: 556.3, ¹H NMR (400 MHz, DMSO) δ 8.24 (s, 1H), 7.88 (d, J=0.9 Hz, 1H), 7.77 (d, J=5.8 Hz, 1H), 7.54 (s, 1H), 7.46 (s, 1H), 7.32 (ddd, J=7.4, 7.4, 1.3 Hz, 1H), 7.28-7.24 (m, 2H), 7.15 (ddd, J=1.3, 1.3, 7.6 Hz, 1H), 7.10 (t, J=5.8 Hz, 1H), 6.33 (dd, J=2.2, 5.8 Hz, 1H), 6.17 (d, J=1.3 Hz, 1H), 4.35 (d, J=5.7 Hz, 2H), 3.49 (s, 4H), 2.41-2.36 (m, 2H), 2.26 (s, 3H), 1.90-1.83 (m, 1H), 1.48-1.43 (m, 1H), 1.39-1.33 (m, 1H), 0.90-0.85 (m, 2H), 0.69-0.64 (m, 2H).

Using a similar procedure to that used for (1S,2S)-2-(3-chlorophenyl)-N-(4-(((6-cyclopropyl-8-(4-methylpiperazin-1-yl)imidazo[1,2-a]pyridin-2-yl)methyl)amino)pyridin-2-yl)cyclopropane-1-carboxamide, the compounds in Table 24 were prepared from (1S,2S)-2-(3-chlorophenyl)cyclopropane-1-carboxamide and an appropriate coupling partner.

TABLE 24

| | | Coupling Partner/ |
|---|---|---|
| Example | Compound | Analytical Data |
| Example 139 | 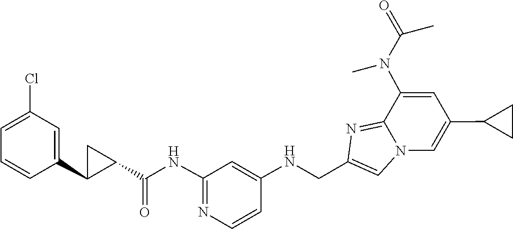 [SMILES: CC(=O)N(C)c1cc(C2CC2)cn2cc(CNc3ccnc(NC(=O)[C@H]4C[C@@H]4c4cccc(Cl)c4)c3)nc12] (1S,2S)-2-(3-chlorophenyl)-N-(4-(((6- cyclopropyl-8-(N- methylacetamido)imidazo[1,2-a]pyridin- 2-yl)methyl)amino)pyridin-2- yl)cyclopropane-1-carboxamide (I-139) | N-(2-(((2-bromopyridin-4- yl)amino)methyl)-6- cyclopropylimidazo[1,2-a]pyridin-8- yl)-N-methylacetamide ESI-MS (M + H)+: 529, ¹H NMR (400 MHz, DMSO) δ 10.35 (s, 1H), 8.40 (s, 1H), 7.82-7.76 (m, 2H), 7.50 (s, 1H), 7.39-7.28 (m, 3H), 7.23-7.18 (m, 2H), 7.11 (s, 1H), 6.38 (dd, J = 2.3, 5.8 Hz, 1H), 4.43 (d, J = 5.8 Hz, 2H), 3.26 (s, 3H), 2.45-2.37 (m, 2H), 2.02-1.94 (m, 1H), 1.84 (s, 3H), 1.52-1.47 (m, 1H), 1.42-1.37 (m, 1H), 1.01-0.95 (m, 2H), 0.77 (q, J = 5.1 Hz, 2H). |
| Example 140 | 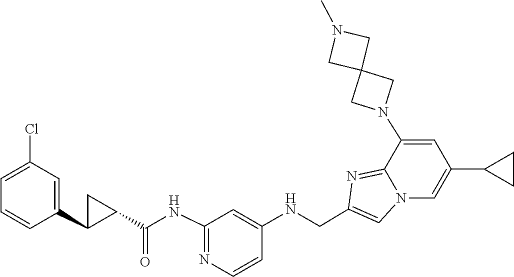 [SMILES: CN1CC2(C1)CN(c1cc(C3CC3)cn3cc(CNc4ccnc(NC(=O)[C@H]5C[C@@H]5c5cccc(Cl)c5)c4)nc13)C2] (1S,2S)-2-(3-chlorophenyl)-N-(4-(((6- cyclopropyl-8-(6-methyl-2,6- diazaspiro[3.3]heptan-2-yl)imidazo[1,2- a]pyridin-2-yl)methyl)amino)pyridin-2- yl)cyclopropane-1-carboxamide (I-140) | 2-bromo-N-((6-cyclopropyl-8-(6- methyl-2,6-diazaspiro[3.3]heptan-2- yl)imidazo[1,2-a]pyridin-2- yl)methyl)pyridin-4-amine ESI-MS (M + H)+: 568.4, ¹H NMR (400 MHz, DMSO) δ 10.32 (s, 1H), 7.77 (d, J = 5.8 Hz, 1H), 7.68 (s, 1H), 7.51 (s, 1H), 7.45 (s, 1H), 7.35-7.30 (m, 1H), 7.26 (d, J = 7.3 Hz, 2H), 7.15 (d, J = 7.8 Hz, 1H), 7.05 (dd, J = 5.6, 5.6 Hz, 1H), 6.33 (dd, J = 2.1, 5.8 Hz, 1H), 5.66 (d, J = 1.1 Hz, 1H), 4.31 (d, J = 5.6 Hz, 2H), 4.16 (s, 4H), 3.30 (s, 2H), 2.42-2.34 (m, 2H), 2.22 (s, 3H), 1.85-1.77 (m, 1H), 1.49-1.43 (m, 1H), 1.39-1.33 (m, 1H), 0.88-0.82 (m, 2H), 0.65- 0.60 (m, 2H). |
| Example 141 | 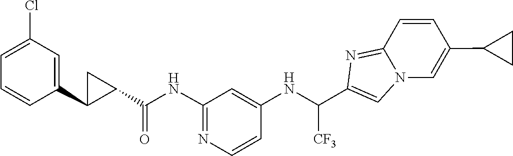 [SMILES: O=C(Nc1cc(NC(c2cn3cc(C4CC4)ccc3n2)C(F)(F)F)ccn1)[C@H]1C[C@@H]1c1cccc(Cl)c1] ((1S,2S)-2-(3-chlorophenyl)-N-(4-((1-(6- cyclopropylimidazo[1,2-a]pyridin-2-yl)- 2,2,2-trifluoroethyl)amino)pyridin-2- yl)cyclopropane-1-carboxamide, formic acid salt (I-141) | 2-bromo-N-(1-(6- cyclopropylimidazo[1,2-a]pyridin-2- yl)-2,2,2-trifluoroethyl)pyridin-4- amine ESI-MS (M + H)+: 526.3, ¹H NMR (400 MHz, DMSO) δ 10.42 (s, 1H), 8.43 (s, 1H), 7.97 (s, 1H), 7.86 (d, J = 5.8 Hz, 1H), 7.66 (s, 1H), 7.48 (d, J = 9.3 Hz, 1H), 7.42 (dd, J = 2.7, 9.5 Hz, 1H), 7.36- 7.31 (m, 1H), 7.27 (d, J = 5.8 Hz, 2H), 7.17 (d, J = 7.8 Hz, 1H), 7.08 (dd, J = 1.8, 9.4 Hz, 1H), 6.65 (dd, J = 2.2, 5.8 Hz, 1H), 5.64 (dd, J = 8.1, 8.1 Hz, 1H), 2.42- 2.34 (m, 3H), 1.99-1.92 (m, 1H), 1.51-1.36 (m, 2H), 0.97-0.92 (m, 2H), 0.73-0.68 (m, 2H). |
| Example 142 | 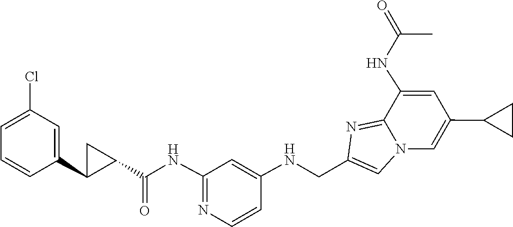 [SMILES: CC(=O)Nc1cc(C2CC2)cn2cc(CNc3ccnc(NC(=O)[C@H]4C[C@@H]4c4cccc(Cl)c4)c3)nc12] (1S,2S)-N-(4-(((8-acetamido-6- cyclopropylimidazo[1,2-a]pyridin-2- yl)methyl)amino)pyridin-2-yl)-2-(3- chlorophenyl)cyclopropane-1- carboxamide, formic acid salt (I-142) | N-(2-(((2-bromopyridin-4- yl)amino)methyl)-6- cyclopropylimidazo[1,2-a]pyridin-8- yl)acetamide ESI-MS (M + H)+: 515.4, ¹H NMR (400 MHz, DMSO) δ 10.38 (s, 1H), 9.93 (s, 1H), 8.08 (s, 1H), 7.84-7.80 (m, 2H), 7.69 (s, 1H), 7.50 (s, 1H), 7.37-7.28 (m, 3H), 7.21-7.12 (m, 2H), 6.36 (dd, J = 1.6, 5.9 Hz, 1H), 4.45 (d, J = 5.6 Hz, 2H), 2.45-2.39 (m, 2H), 2.25 (s, 3H), 1.98-1.91 (m, 1H), 1.53-1.46 (m, 1H), 1.43-1.38 (m, 1H), 0.98-0.92 (m, 2H), 0.69-0.64 (m, 2H). |

### Example 143

### Synthesis of rac-(1S*,2S*)-2-(5-chloro-2-cyanophenyl)-N-(4-(((6-cyclopropyl-8-(4-methyl piperazin-1-yl)imidazo[1,2-a]pyridin-2-yl)methyl)amino)pyridin-2-yl)cyclopropane-1-carboxamide (I-143)

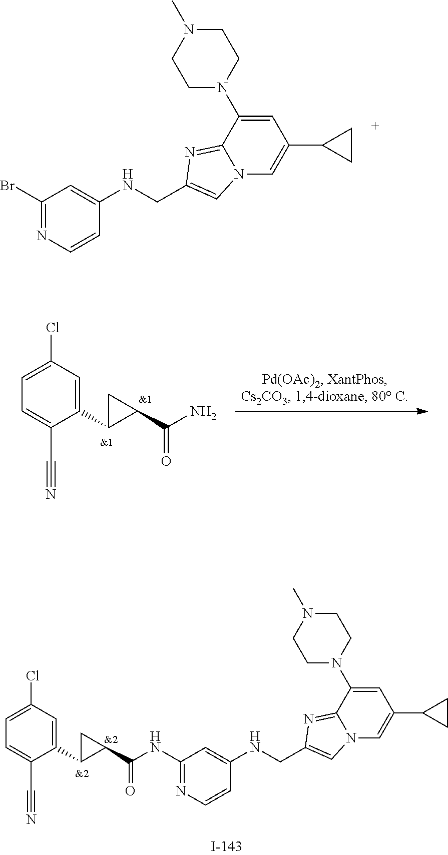 [SMILES: CN1CCN(c2cc(C3CC3)cn3cc(CNc4ccnc(Br)c4)nc23)CC1 ; N#Cc1ccc(Cl)cc1[C@@H]1C[C@H]1C(N)=O ; "Pd(OAc)2, XantPhos,Cs2CO3, 1,4-dioxane, 80° C." ; CN1CCN(c2cc(C3CC3)cn3cc(CNc4ccnc(NC(=O)[C@@H]5C[C@H]5c5cc(Cl)ccc5C#N)c4)nc23)CC1]

A suspension of 2-bromo-N-((6-cyclopropyl-8-(4-methylpiperazin-1-yl)imidazo[1,2-a]pyridin-2-yl)methyl)pyridin-4-amine (56 mg, 0.13 mmol), rac-(1S*,2S*)-2-(5-chloro-2-cyanophenyl)cyclopropane-1-carboxamide (31 mg, 0.14 mmol), XantPhos (15 mg, 0.03 mmol), and Cs₂CO₃ (83 mg, 0.25 mmol) in 1,4-dioxane (3.0 mL) was degassed for 5 min. Pd(OAc)₂ (2.8 mg, 0.01 mmol) was added and the reaction mixture was stirred at 80° C. for 16 h. The reaction mixture was cooled to room temperature, filtered through Celite®, and concentrated in vacuo. The residue was purified by column chromatography on silica gel, eluting with a gradient of 0-20% (7N NH₃ in MeOH) in DCM. The residue was further purified by column chromatography on silica gel, eluting with 2-2.5% (7N NH₃ in MeOH) in DCM. The residue was further purified by preparative HPLC to give of the title compound (10 mg, 14%). ESI-MS (M+H)+: 581.5, ¹H NMR (400 MHz, DMSO) δ 10.38 (s, 1H), 7.89-7.86 (m, 2H), 7.78 (d, J=5.8 Hz, 1H), 7.56-7.51 (m, 2H), 7.48 (s, 1H), 7.40 (d, J=2.0 Hz, 1H), 7.13 (dd, J=5.6, 5.6 Hz, 1H), 6.34 (dd, J=2.1, 5.8 Hz, 1H), 6.16 (s, 1H), 4.35 (d, J=5.8 Hz, 2H), 3.49-3.49 (m, 4H), 2.61-2.55 (m, 2H), 2.25 (s, 3H), 1.90-1.82 (m, 1H), 1.55 (dd, J=7.3, 7.3 Hz, 2H), 0.90-0.84 (m, 2H), 0.69-0.64 (m, 2H).

### Synthesis of 1-(2-(((6-chloropyrimidin-4-yl)amino)methyl)-6-cyclopropylimidazo[1,2-a]pyridin-8-yl)pyrrolidin-2-one

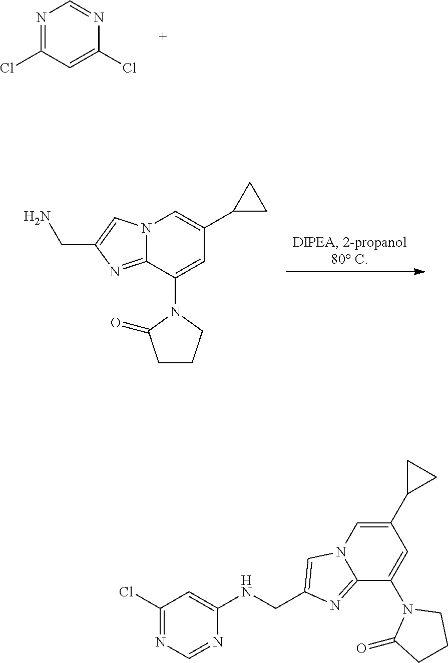 [SMILES: Clc1cc(Cl)ncn1 ; NCc1cn2cc(C3CC3)cc(N3CCCC3=O)c2n1 ; "DIPEA, 2-propanol80° C." ; O=C1CCCN1c1cc(C2CC2)cn2cc(CNc3cc(Cl)ncn3)nc12]

A solution of 2,4-dichloropyrimidine (0.12 g, 0.84 mmol), 1-(2-(aminomethyl)-6-cyclo propylimidazo[1,2-a]pyridin-8-yl)pyrrolidin-2-one (0.25 g, 0.92 mmol) and DIPEA (0.44 mL, 2.52 mmol) in 2-propanol (5 mL) was heated to 70° C. for 1 hour. The mixture was cooled to RT, diluted with H₂O (20 mL), then extracted with DCM (3×10 mL). The combined organics were dried over MgSO₄, then concentrated in vacuo. The residue was triturated with diethyl ether to give the crude product which was used without further purification (0.27 g, 84%). ESI-MS (M+H)+: 383, 1H NMR: ¹H NMR (400 MHz, DMSO) δ 8.35 (s, 1H), 8.30 (d, J=1.0 Hz, 1H), 8.25 (s, 1H), 7.77 (s, 1H), 7.18 (s, 1H), 6.69 (s, 1H), 4.67-4.66 (m, 2H), 4.19 (dd, J=7.1, 7.1 Hz, 2H), 2.02-1.93 (m, 1H), 1.32 (dd, J=7.2, 7.2 Hz, 4H), 0.98 (ddd, J=4.4, 6.4, 8.4 Hz, 2H), 0.72-0.67 (m, 2H).

The compounds in Table 25 were synthesized using a similar procedure to that used for 1-(2-(((6-chloropyrimidin-4-yl)amino)methyl)-6-cyclopropylimidazo[1,2-a]pyridin-8-yl)pyrrolidin-2-one from a suitable di-halo-pyrimidine or pyridine and an appropriate coupling partner.

TABLE 25

| | Coupling Partner/ |
|---|---|
| Structure | Analytical Data |
| 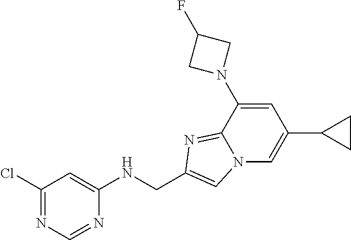 [SMILES: FC1CN(c2cc(C3CC3)cn3cc(CNc4cc(Cl)ncn4)nc23)C1] 6-chloro-N-((6-cyclopropyl-8-(3-fluoroazetidin- 1-yl)imidazo[1,2-a]pyridin-2- yl)methyl)pyrimidin-4-amine | (6-cyclopropyl-8-(3-fluoroazetidin-1- yl)imidazo[1,2-a]pyridin-2-yl)methanamine ESI-MS (M + H)+: 373.2, |
| 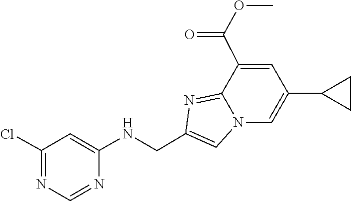 [SMILES: COC(=O)c1cc(C2CC2)cn2cc(CNc3cc(Cl)ncn3)nc12] methyl 2-(((6-chloropyrimidin-4- yl)amino)methyl)-6-cyclopropylimidazo[1,2- a]pyridine-8-carboxylate | methyl 2-(((6-chloropyrimidin-4- yl)amino)methyl)-6-cyclopropylimidazo[1,2- a]pyridine-8-carboxylate ESI-MS (M + H)+: 358.2 ¹H NMR (400 MHz, DMSO) δ 8.30-8.28 (m, 1H), 8.14 (s, 1H), 7.75 (s, 1H), 7.59 (s, 1H), 6.65-6.61 (m, 1H), 5.78-5.74 (m, 1H), 5.58-5.38 (m, 1H), 4.58- 4.58 (m, 1H), 4.51-4.35 (m, 2H), 4.19-4.09 (m, 2H), 1.87-1.80 (m, 1H), 0.90-0.84 (m, 2H), 0.68-0.62 (m, 2H). |
| 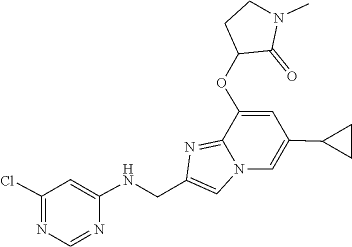 [SMILES: CN1CCC(Oc2cc(C3CC3)cn3cc(CNc4cc(Cl)ncn4)nc23)C1=O] 3-((2-(((6-chloropyrimidin-4-yl)amino)methyl)- 6-cyclopropylimidazo[1,2-a]pyridin-8-yl)oxy)-1- methylpyrrolidin-2-one | 3-((2-(aminomethyl)-6- cyclopropylimidazo[1,2-a]pyridin-8-yl)oxy)- 1-methylpyrrolidin-2-one ESI-MS (M + H)+: 413.2 |
| 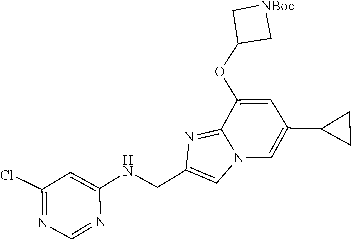 [SMILES: CC(C)(C)OC(=O)N1CC(Oc2cc(C3CC3)cn3cc(CNc4cc(Cl)ncn4)nc23)C1] tert-butyl 3-((2-(((6-chloropyrimidin-4- yl)amino)methyl)-6-cyclopropylimidazo[1,2- a]pyridin-8-yl)oxy)azetidine-1-carboxylate | tert-butyl 3-((2-(((6-chloropyrimidin-4- yl)amino)methyl)-6-cyclopropylimidazo[1,2- a]pyridin-8-yl)oxy)azetidine-1-carboxylate ¹H NMR (400 MHz, CDCl₃): δ, ppm, 8.39 (1H, s), 8.04 (dd, J = 0.7, 1.8 Hz, 1H), 7.75 (d, J = 1.8 Hz, 1H), 7.51 (s, 1H), 6.43 (d, J = 0.9 Hz, 1H), 6.14 (br s, 1H), 4.75 (br s, 2H), 4.03 (s, 3H), 1.97-1.90 (m, 1H), 1.05-1.00 (m, 2H), 0.75-0.69 (m, 2H). |
| 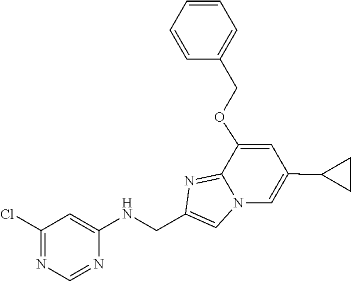 [SMILES: Clc1cc(NCc2cn3cc(C4CC4)cc(OCc4ccccc4)c3n2)ncn1] N-((8-(benzyloxy)-6-cyclopropylimidazo[1,2- a]pyridin-2-yl)methyl)-6-chloropyrimidin-4- amine | (8-(benzyloxy)-6-cyclopropylimidazo[1,2- a]pyridin-2-yl)methanamine ESI-MS (M + H)+: 406.3 ¹H NMR (400 MHz, CDCl₃) δ 8.38 (s, 1H), 7.55 (s, 1H), 7.39 (s, 1H), 6.75 (s, 1H), 6.42 (s, 1H), 6.04-6.03 (m, 1H), 5.24-5.19 (m, 1H), 4.65 (s, 2H), 3.63- 3.56 (m, 1H), 3.44-3.37 (m, 1H), 2.93 (s, 3H), 2.65-2.55 (m, 1H), 2.47-2.38 (m, 1H), 1.90- 1.83 (m, 1H), 0.96-0.91 (m, 2H), 0.70-0.66 (m, 2H). |
| 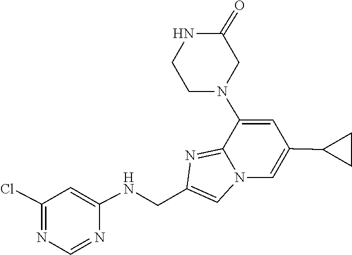 [SMILES: O=C1CN(c2cc(C3CC3)cn3cc(CNc4cc(Cl)ncn4)nc23)CCN1] 4-(2-(((6-chloropyrimidin-4-yl)amino)methyl)-6- cyclopropylimidazo[1,2-a]pyridin-8-yl)piperazin- 2-one | 4-(2-(aminomethyl)-6- cyclopropylimidazo[1,2-a]pyridin-8- yl)piperazin-2-one ESI-MS (M + H)+: 398.3 ¹H NMR (400 MHz, DMSO) δ 8.30 (s, 1H), 8.21 (s, 1H), 7.97 (s, 1H), 7.69 (s, 1H), 6.63 (s, 1H), 6.16 (s, 1H), 4.60-4.60 (m, 2H), 4.36 (dd, J = 7.2, 7.2 Hz, 2H), 3.86 (dd, J = 2.6, 9.3 Hz, 2H), 1.41 (s, 9H), 0.94-0.87 (m, 2H), 0.71-0.66 (m, 2H). |
| 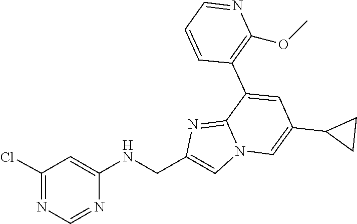 [SMILES: COc1ncccc1-c1cc(C2CC2)cn2cc(CNc3cc(Cl)ncn3)nc12] 4-(2-(((6-chloropyrimidin-4-yl)amino)methyl)-6- cyclopropylimidazo[1,2-a]pyridin-8-yl)piperazin- 2-one | (6-cyclopropyl-8-(2-methoxypyridin-3- yl)imidazo[1,2-a]pyridin-2-yl)methanamine ESI-MS (M + H)+: 406.3 |
| 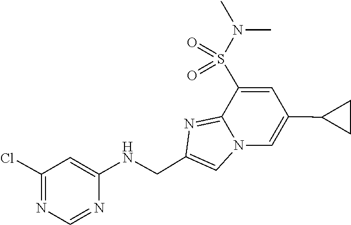 [SMILES: CN(C)S(=O)(=O)c1cc(C2CC2)cn2cc(CNc3cc(Cl)ncn3)nc12] 2-(((6-chloropyrimidin-4-yl)amino)methyl)-6- cyclopropyl-N,N-dimethylimidazo[1,2- a]pyridine-8-sulfonamide | 2-(aminomethyl)-6-cyclopropyl-N,N- dimethylimidazo[1,2-a]pyridine-8- sulfonamide ¹H NMR (400 MHz, DMSO) δ 8.34 (s, 1H), 8.24 (s, 1H), 7.99 (s, 1H), 7.72 (s, 1H), 7.57- 7.53 (m, 2H), 7.50-7.41 (m, 3H), 6.67 (s, 1H), 6.52 (d, J = 1.0 Hz, 1H), 5.30 (s, 2H), 4.63- 4.62 (m, 2H), 1.97-1.89 (m, 1H), 0.97-0.91 (m, 2H), 0.75-0.69 (m, 2H). |
| 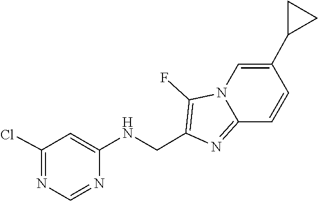 [SMILES: Fc1c(CNc2cc(Cl)ncn2)nc2ccc(C3CC3)cn12] 6-chloro-N-((6-cyclopropyl-3-fluoroimidazo[1,2- a]pyridin-2-yl)methyl)pyrimidin-4-amine | (6-cyclopropyl-3-fluoroimidazo[1,2- a]pyridin-2-yl)methanamine ESI-MS (M + H)+: 318.4 |
| 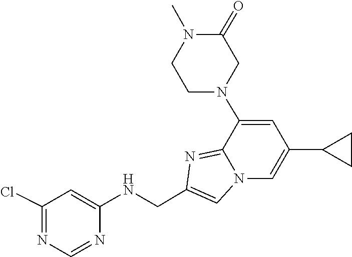 [SMILES: CN1CCN(c2cc(C3CC3)cn3cc(CNc4cc(Cl)ncn4)nc23)CC1=O] 4-(2-(((6-chloropyrimidin-4-yl)amino)methyl)-6- cyclopropylimidazo[1,2-a]pyridin-8-yl)-1- methylpiperazin-2-one | 4-(2-(((6-chloropyrimidin-4- yl)amino)methyl)-6-cyclopropylimidazo[1,2- a]pyridin-8-yl)-1-methylpiperazin-2-one ESI-MS (M + H)+: 412.4 |
| 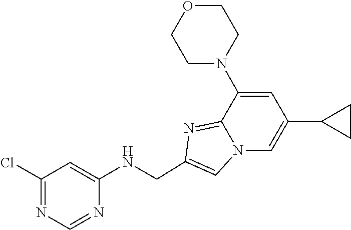 [SMILES: Clc1cc(NCc2cn3cc(C4CC4)cc(N4CCOCC4)c3n2)ncn1] 6-chloro-N-((6-cyclopropyl-8- morpholinoimidazo[1,2-a]pyridin-2- yl)methyl)pyrimidin-4-amine | (6-cyclopropyl-8-morpholinoimidazo[1,2- a]pyridin-2-yl)methanamine ESI-MS (M + H)+: 385.1 |
| 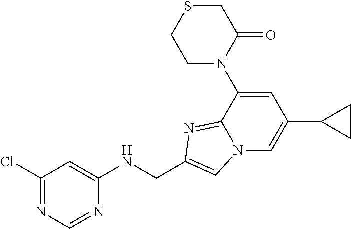 [SMILES: O=C1CSCCN1c1cc(C2CC2)cn2cc(CNc3cc(Cl)ncn3)nc12] 4-(2-(((6-chloropyrimidin-4-yl)amino)methyl)-6- cyclopropylimidazo[1,2-a]pyridin-8- yl)thiomorpholin-3-one | 4-(2-(aminomethyl)-6- cyclopropylimidazo[1,2-a]pyridin-8- yl)thiomorpholin-3-one ESI-MS (M + H)+: 415 |
| 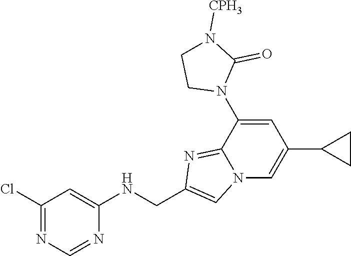 1-(2-(((6-chloropyrimidin-4-yl)amino)methyl)-6- cyclopropylimidazo[1,2-a]pyridin-8-yl)-3- tritylimidazolidin-2-one | 1-(2-(aminomethyl)-6- cyclopropylimidazo[1,2-a]pyridin-8-yl)-3- tritylimidazolidin-2-one ¹H NMR (400 MHz, CDCl₃) δ 8.37 (d, J = 9.2 Hz, 1H), 8.22 (dd, J = 1.9, 5.0 Hz, 1H), 8.06 (dd, J = 1.9, 7.4 Hz, 1H), 7.87-7.86 (m, 1H), 7.48 (s, 1H), 7.15 (d, J = 1.5 Hz, 1H), 7.05- 6.99 (m, 1H), 6.41 (s, 1H), 5.97-5.97 (m, 1H), 4.63-4.63 (m, 2H), 3.95 (s, 3H), 1.97-1.89 (m, 1H), 1.02-0.96 (m, 2H), 0.73-0.68 (m, 2H). |
| 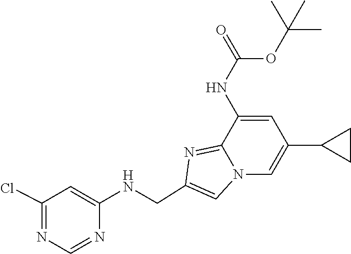 [SMILES: CC(C)(C)OC(=O)Nc1cc(C2CC2)cn2cc(CNc3cc(Cl)ncn3)nc12] tert-butyl (2-(((6-chloropyrimidin-4- yl)amino)methyl)-6-cyclopropylimidazo[1,2- a]pyridin-8-yl)carbamate | tert-butyl (2-(aminomethyl)-6- cyclopropylimidazo[1,2-a]pyridin-8- yl)carbamate ESI-MS (M + H)+: 414.3 |
| 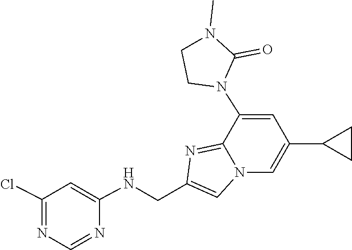 [SMILES: CN1CCN(c2cc(C3CC3)cn3cc(CNc4cc(Cl)ncn4)nc23)C1=O] 1-(2-(((6-chloropyrimidin-4-yl)amino)methyl)-6- cyclopropylimidazo[1,2-a]pyridin-8-yl)-3- methylimidazolidin-2-one | tert-butyl (2-(aminomethyl)-6- cyclopropylimidazo[1,2-a]pyridin-8- yl)carbamate ¹H NMR (400 MHz, DMSO) δ 8.57 (s, 1H), 8.28 (dd, J = 6.3, 6.3 Hz, 2H), 7.84 (s, 1H), 7.51 (s, 1H), 6.63 (s, 1H), 4.68-4.68 (m, 2H), 2.84 (s, 6H), 2.09-2.01 (m, 1H), 1.01-0.95 (m, 2H), 0.75-0.69 (m, 2H). |
| 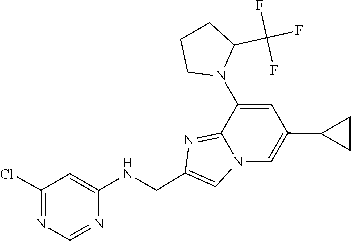 [SMILES: FC(F)(F)C1CCCN1c1cc(C2CC2)cn2cc(CNc3cc(Cl)ncn3)nc12] 6-chloro-N-((6-cyclopropyl-8-(2- (trifluoromethyl)pyrrolidin-1-yl)imidazo[1,2- a]pyridin-2-yl)methyl)pyrimidin-4-amine | (6-cyclopropyl-8-(2- (trifluoromethyl)pyrrolidin-1-yl)imidazo[1,2- a]pyridin-2-yl)methanamine ESI-MS (M + H)+: 436.2 |
| 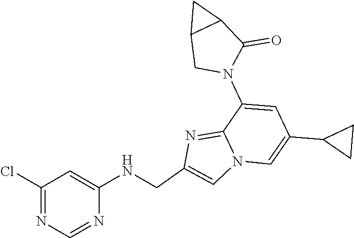 [SMILES: O=C1C2CC2CN1c1cc(C2CC2)cn2cc(CNc3cc(Cl)ncn3)nc12] 3-(2-(((6-chloropyrimidin-4-yl)amino)methyl)-6- cyclopropylimidazo[1,2-a]pyridin-8-yl)-3- azabicyclo[3.1.0]hexan-2-one | 3-(2-(aminomethyl)-6- cyclopropylimidazo[1,2-a]pyridin-8-yl)-3- azabicyclo[3.1.0]hexan-2-one ¹H NMR (400 MHz, CDCl₃) δ 8.40 (s, 1H), 7.66 (s, 1H), 7.35 (d, J = 9.6 Hz, 1H), 6.93 (dd, J = 1.6, 9.5 Hz, 1H), 6.47 (s, 1H), 5.86-5.83 (m, 1H), 4.68-4.67 (m, 2H), 1.95-1.88 (m, 1H), 1.02-0.97 (m, 2H), 0.73-0.68 (m, 2H). |
| 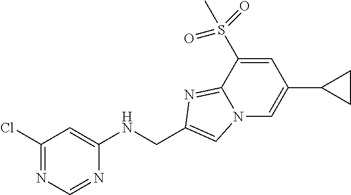 [SMILES: CS(=O)(=O)c1cc(C2CC2)cn2cc(CNc3cc(Cl)ncn3)nc12] 6-chloro-N-((6-cyclopropyl-8- (methylsulfonyl)imidazo[1,2-a]pyridin-2- yl)methyl)pyrimidin-4-amine | (6-cyclopropyl-8- (methylsulfonyl)imidazo[1,2-a]pyridin-2- yl)methanamine ESI-MS (M + H)+: 378.3 |
| 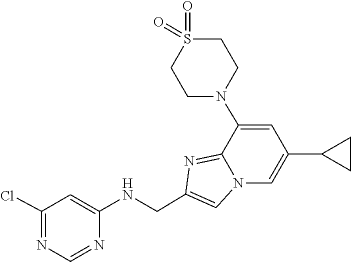 [SMILES: O=S1(=O)CCN(c2cc(C3CC3)cn3cc(CNc4cc(Cl)ncn4)nc23)CC1] 4-(2-(((6-chloropyrimidin-4-yl)amino)methyl)-6- cyclopropylimidazo[1,2-a]pyridin-8- yl)thiomorpholine 1,1-dioxide | 4-(2-(aminomethyl)-6- cyclopropylimidazo[1,2-a]pyridin-8- yl)thiomorpholine 1,1-dioxide ESI-MS (M + H)+: 433.3 |
| 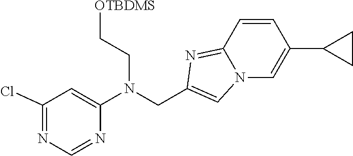 [SMILES: CC(C)(C)[Si](C)(C)OCCN(Cc1cn2cc(C3CC3)ccc2n1)c1cc(Cl)ncn1] N-(2-((tert-butyldimethylsilyl)oxy)ethyl)-6- chloro-N-((6-cyclopropylimidazo[1,2-a]pyridin- 2-yl)methyl)pyrimidin-4-amine | 2-((tert-butyldimethylsilyl)oxy)-N-((6- cyclopropylimidazo[1,2-a]pyridin-2- yl)methyl)ethan-1-amine ESI-MS (M + H)+: 458.4 |
| 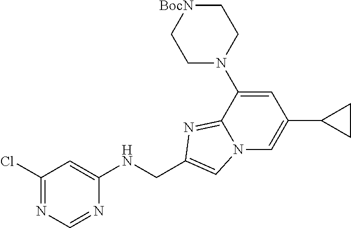 [SMILES: CC(C)(C)OC(=O)N1CCN(c2cc(C3CC3)cn3cc(CNc4cc(Cl)ncn4)nc23)CC1] tert-butyl 4-(2-(((6-chloropyrimidin-4- yl)amino)methyl)-6-cyclopropylimidazo[1,2- a]pyridin-8-yl)piperazine-1-carboxylate | tert-butyl 4-(2-(aminomethyl)-6- cyclopropylimidazo[1,2-a]pyridin-8- yl)piperazine-1-carboxylate ESI-MS (M + H)+: 484.2 |
| 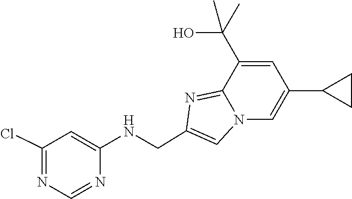 [SMILES: CC(C)(O)c1cc(C2CC2)cn2cc(CNc3cc(Cl)ncn3)nc12] 2-(2-(((6-chloropyrimidin-4-yl)amino)methyl)-6- cyclopropylimidazo[1,2-a]pyridin-8-yl)propan-2- ol | 2-(2-(aminomethyl)-6- cyclopropylimidazo[1,2-a]pyridin-8- yl)propan-2-ol ESI-MS (M + H)+: 358 |
| 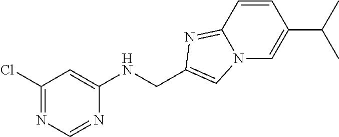 [SMILES: CC(C)c1ccc2nc(CNc3cc(Cl)ncn3)cn2c1] 6-chloro-N-((6-isopropylimidazo[1,2-a]pyridin- 2-yl)methyl)pyrimidin-4-amine | (6-isopropylimidazo[1,2-a]pyridin-2- yl)methanamine ESI-MS (M + H)+: 302.1 |
| 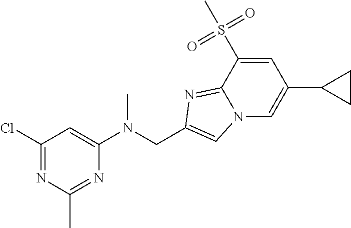 [SMILES: Cc1nc(Cl)cc(N(C)Cc2cn3cc(C4CC4)cc(S(C)(=O)=O)c3n2)n1] 6-chloro-N-((6-cyclopropyl-8- (methylsulfonyl)imidazo[1,2-a]pyridin-2- yl)methyl)-N,2-dimethylpyrimidin-4-amine | 1-(6-cyclopropyl-8- (methylsulfonyl)imidazo[1,2-a]pyridin-2-yl)- N-methylmethanamine ESI-MS [M + H]⁺: 406.2 |
| 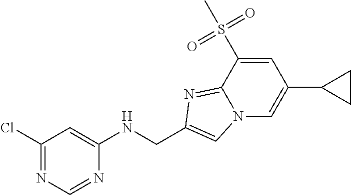 [SMILES: CS(=O)(=O)c1cc(C2CC2)cn2cc(CNc3cc(Cl)ncn3)nc12] 6-chloro-N-((6-cyclopropyl-8- (methylsulfonyl)imidazo[1,2-a]pyridin-2- yl)methyl)pyrimidin-4-amine | (6-cyclopropyl-8- (methylsulfonyl)imidazo[1,2-a]pyridin-2- yl)methanamine ESI-MS [M + H]⁺: 378.2. ¹H NMR (400 MHz, DMSO) δ 8.63 (s, 1H), 8.32-8.30 (m, 2H), 7.87 (s, 1H), 7.57 (d, J = 1.5 Hz, 1H), 6.65 (s, 1H), 4.69 (s, 2H), 3.53 (s, 3H), 2.10-2.04 (m, 1H), 1.00-0.95 (m, 2H), 0.73-0.69 (m, 2H). |
| 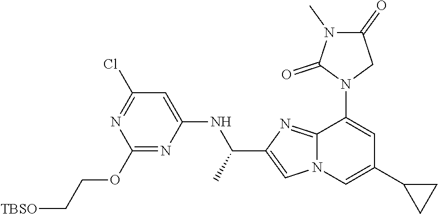 [SMILES: C[C@H](Nc1cc(Cl)nc(OCCO[Si](C)(C)C(C)(C)C)n1)c1cn2cc(C3CC3)cc(N3CC(=O)N(C)C3=O)c2n1] (R)-1-(2-(1-((2-(2-((tert- butyldimethylsilyl)oxy)ethoxy)-6- chloropyrimidin-4-yl)amino)ethyl)-6- cyclopropylimidazo[1,2-a]pyridin-8-yl)-3- methylimidazolidine-2,4-dione | (S)-1-(2-(1-aminoethyl)-6- cyclopropylimidazo[1,2-a]pyridin-8-yl)-3- methylimidazolidine-2,4-dione ESI-MS [M + H]⁺: 600.2 |
| 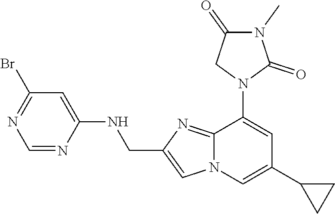 [SMILES: CN1C(=O)CN(c2cc(C3CC3)cn3cc(CNc4cc(Br)ncn4)nc23)C1=O] 1-(2-(((6-bromopyrimidin-4-yl)amino)methyl)-6- cyclopropylimidazo[1,2-a]pyridin-8-yl)-3- methylimidazolidine-2,4-dione | 1-(2-(aminomethyl)-6- cyclopropylimidazo[1,2-a]pyridin-8-yl)-3- methylimidazolidine-2,4-dione ESI-MS [M + H]⁺: 456.2 |
| 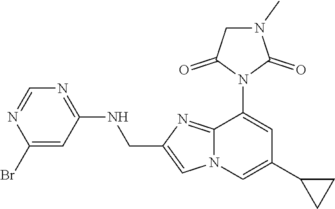 [SMILES: CN1CC(=O)N(c2cc(C3CC3)cn3cc(CNc4cc(Br)ncn4)nc23)C1=O] 3-(2-(((6-bromopyrimidin-4-yl)amino)methyl)-6- cyclopropylimidazo[1,2-a]pyridin-8-yl)-1- methylimidazolidine-2,4-dione | 3-(2-(aminomethyl)-6- cyclopropylimidazo[1,2-a]pyridin-8-yl)-1- methylimidazolidine-2,4-dione ESI-MS [M + H]⁺: 456.2 |
| 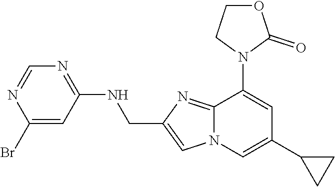 [SMILES: O=C1OCCN1c1cc(C2CC2)cn2cc(CNc3cc(Br)ncn3)nc12] 3-(2-(((6-bromopyrimidin-4-yl)amino)methyl)-6- cyclopropylimidazo[1,2-a]pyridin-8- yl)oxazolidin-2-one | 3-(2-(aminomethyl)-6- cyclopropylimidazo[1,2-a]pyridin-8- yl)oxazolidin-2-one ESI-MS [M + H]+: 429.2. ¹H NMR (400 MHz, DMSO) δ 8.25-8.24 (m, 3H), 7.75 (s, 1H), 7.23 (s, 1H), 6.82 (s, 1H), 4.66-4.51 (m, 2H), 4.52-4.41 (m, 4H), 1.97-1.89 (m, 1H), 0.98-0.90 (m, 2H), 0.68-0.62 (m, 2H). |
| 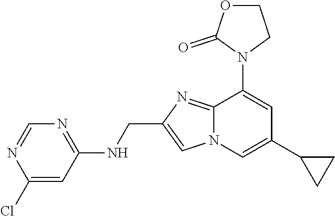 [SMILES: O=C1OCCN1c1cc(C2CC2)cn2cc(CNc3cc(Cl)ncn3)nc12] 3-(2-(((6-chloropyrimidin-4-yl)amino)methyl)-6- cyclopropylimidazo[1,2-a]pyridin-8- yl)oxazolidin-2-one | 3-(2-(aminomethyl)-6- cyclopropylimidazo[1,2-a]pyridin-8- yl)oxazolidin-2-one ESI-MS [M + H]⁺: 385.2 |
| 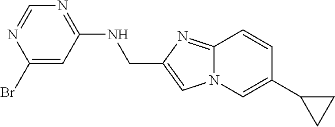 [SMILES: Brc1cc(NCc2cn3cc(C4CC4)ccc3n2)ncn1] 6-bromo-N-((6-cyclopropylimidazo[1,2- a]pyridin-2-yl)methyl)pyrimidin-4-amine | (6-cyclopropylimidazo[1,2-a]pyridin-2- yl)methanamine ESI-MS [M + H]⁺: 344.2 |
| 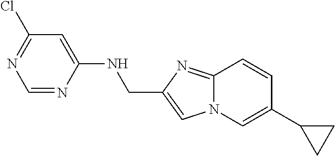 [SMILES: Clc1cc(NCc2cn3cc(C4CC4)ccc3n2)ncn1] 6-chloro-N-((6-cyclopropylimidazo[1,2- a]pyridin-2-yl)methyl)pyrimidin-4-amine | (6-cyclopropylimidazo[1,2-a]pyridin-2- yl)methanamine ESI-MS [M + H]⁺: 300.2 |
| 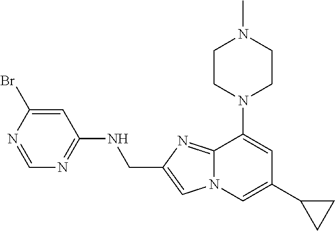 [SMILES: CN1CCN(c2cc(C3CC3)cn3cc(CNc4cc(Br)ncn4)nc23)CC1] 6-bromo-N-((6-cyclopropyl-8-(4- methylpiperazin-1-yl)imidazo[1,2-a]pyridin-2- yl)methyl)pyrimidin-4-amine | (6-cyclopropyl-8-(4-methylpiperazin-1- yl)imidazo[1,2-a]pyridin-2-yl)methanamine ESI-MS [M + H]⁺: 442.2 |
| 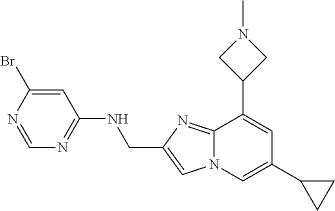 [SMILES: CN1CC(c2cc(C3CC3)cn3cc(CNc4cc(Br)ncn4)nc23)C1] 6-bromo-N-((6-cyclopropyl-8-(1-methylazetidin- 3-yl)imidazo[1,2-a]pyridin-2- yl)methyl)pyrimidin-4-amine | (6-cyclopropyl-8-(1-methylazetidin-3- yl)imidazo[1,2-a]pyridin-2-yl)methanamine ESI-MS [M + H]⁺: 413.2 |
| 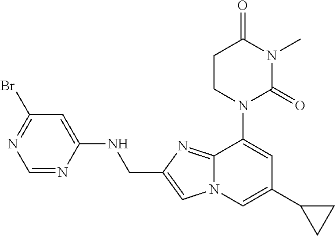 [SMILES: CN1C(=O)CCN(c2cc(C3CC3)cn3cc(CNc4cc(Br)ncn4)nc23)C1=O] 1-(2-(((6-bromopyrimidin-4-yl)amino)methyl)-6- cyclopropylimidazo[1,2-a]pyridin-8-yl)-3- methyldihydropyrimidine-2,4(1H,3H)-dione | 1-(2-(aminomethyl)-6- cyclopropylimidazo[1,2-a]pyridin-8-yl)-3- methyldihydropyrimidine-2,4(1H,3H)-dione ESI-MS [M + H]⁺: 470.2 |
| 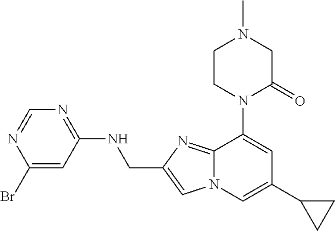 [SMILES: CN1CCN(c2cc(C3CC3)cn3cc(CNc4cc(Br)ncn4)nc23)C(=O)C1] 1-(2-(((6-bromopyrimidin-4-yl)amino)methyl)-6- cyclopropylimidazo[1,2-a]pyridin-8-yl)-4- methylpiperazin-2-one | 1-(2-(aminomethyl)-6- cyclopropylimidazo[1,2-a]pyridin-8-yl)-4- methylpiperazin-2-one ESI-MS [M + H]⁺: 456.2 |
| 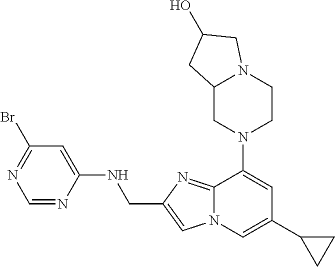 [SMILES: OC1CC2CN(c3cc(C4CC4)cn4cc(CNc5cc(Br)ncn5)nc34)CCN2C1] 2-(2-(((6-bromopyrimidin-4-yl)amino)methyl)-6- cyclopropylimidazo[1,2-a]pyridin-8- yl)octahydropyrrolo[1,2-a]pyrazin-7-ol | 2-(2-(aminomethyl)-6- cyclopropylimidazo[1,2-a]pyridin-8- yl)octahydropyrrolo[1,2-a]pyrazin-7-ol ESI-MS [M + H]⁺: 484.2 |
| 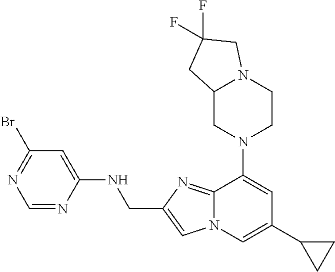 [SMILES: FC1(F)CC2CN(c3cc(C4CC4)cn4cc(CNc5cc(Br)ncn5)nc34)CCN2C1] 6-bromo-N-((6-cyclopropyl-8-(7,7- difluorohexahydropyrrolo[1,2-a]pyrazin-2(1H)- yl)imidazo[1,2-a]pyridin-2-yl)methyl)pyrimidin- 4-amine | (6-cyclopropyl-8-(7,7- difluorohexahydropyrrolo[1,2-a]pyrazin- 2(1H)-yl)imidazo[1,2-a]pyridin-2- yl)methanamine ESI-MS [M + H]⁺: 504.2 |
| 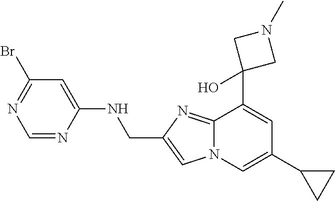 [SMILES: CN1CC(O)(c2cc(C3CC3)cn3cc(CNc4cc(Br)ncn4)nc23)C1] 3-(2-(((6-bromopyrimidin-4-yl)amino)methyl)-6- cyclopropylimidazo[1,2-a]pyridin-8-yl)-1- methylazetidin-3-ol | 3-(2-(aminomethyl)-6- cyclopropylimidazo[1,2-a]pyridin-8-yl)-1- methylazetidin-3-ol ESI-MS [M + H]⁺: 429.2 |
| 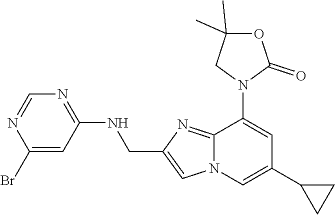 [SMILES: CC1(C)CN(c2cc(C3CC3)cn3cc(CNc4cc(Br)ncn4)nc23)C(=O)O1] 3-(2-(((6-bromopyrimidin-4-yl)amino)methyl)-6- cyclopropylimidazo[1,2-a]pyridin-8-yl)-5,5- dimethyloxazolidin-2-one | 3-(2-(aminomethyl)-6- cyclopropylimidazo[1,2-a]pyridin-8-yl)-5,5- dimethyloxazolidin-2-one ESI-MS [M + H]⁺: 457.2 |
| 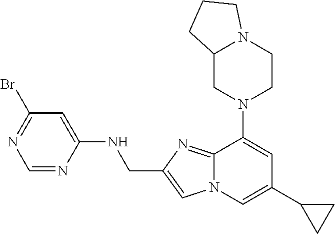 [SMILES: Brc1cc(NCc2cn3cc(C4CC4)cc(N4CCN5CCCC5C4)c3n2)ncn1] 6-bromo-N-((6-cyclopropyl-8- (hexahydropyrrolo[1,2-a]pyrazin-2(1H)- yl)imidazo[1,2-a]pyridin-2-yl)methyl)pyrimidin- 4-amine | (6-cyclopropyl-8-(hexahydropyrrolo[1,2- a]pyrazin-2(1H)-yl)imidazo[1,2-a]pyridin-2- yl)methanamine ESI-MS [M + H]⁺: 468.2 |
| 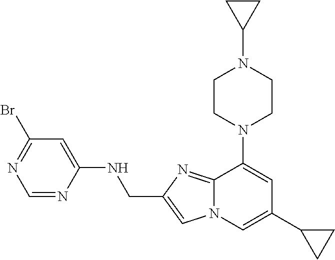 [SMILES: Brc1cc(NCc2cn3cc(C4CC4)cc(N4CCN(C5CC5)CC4)c3n2)ncn1] 6-bromo-N-((6-cyclopropyl-8-(4- cyclopropylpiperazin-1-yl)imidazo[1,2-a]pyridin- 2-yl)methyl)pyrimidin-4-amine | (6-cyclopropyl-8-(4-cyclopropylpiperazin-1- yl)imidazo[1,2-a]pyridin-2-yl)methanamine ESI-MS [M + H]⁺: 468.2 |
| 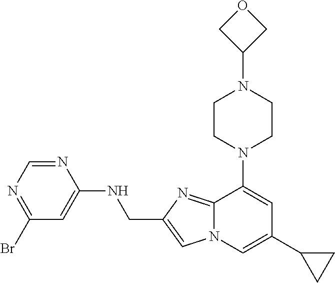 [SMILES: Brc1cc(NCc2cn3cc(C4CC4)cc(N4CCN(C5COC5)CC4)c3n2)ncn1] 6-bromo-N-((6-cyclopropyl-8-(4-(oxetan-3- yl)piperazin-1-yl)imidazo[1,2-a]pyridin-2- yl)methyl)pyrimidin-4-amine | (6-cyclopropyl-8-(4-(oxetan-3-yl)piperazin-1- yl)imidazo[1,2-a]pyridin-2-yl)methanamine ESI-MS [M + H]⁺: 484.2 |
| 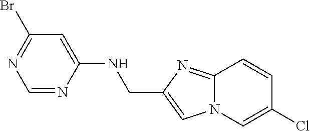 [SMILES: Clc1ccc2nc(CNc3cc(Br)ncn3)cn2c1] 6-bromo-N-((6-chloroimidazo[1,2-a]pyridin-2- yl)methyl)pyrimidin-4-amine | (6-chloroimidazo[1,2-a]pyridin-2- yl)methanamine ESI-MS [M + H]⁺: 338.2 |
| 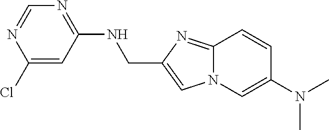 [SMILES: CN(C)c1ccc2nc(CNc3cc(Cl)ncn3)cn2c1] 2-(((6-chloropyrimidin-4-yl)amino)methyl)-N,N- dimethylimidazo[1,2-a]pyridin-6-amine | (6-(N,N-dimethylamino)-imidazo[1,2- a]pyridin-2-yl)methanamine ESI-MS [M + H]⁺: 303.2 |
| 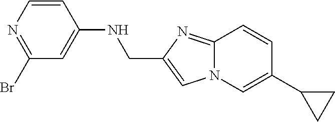 [SMILES: Brc1cc(NCc2cn3cc(C4CC4)ccc3n2)ccn1] 2-bromo-N-((6-cyclopropylimidazo[1,2- a]pyridin-2-yl)methyl)pyridin-4-amine | (6-cyclopropylimidazo[1,2-a]pyridin-2- yl)methanamine ESI-MS [M + H]⁺: 343.2 |
| 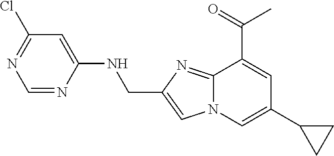 [SMILES: CC(=O)c1cc(C2CC2)cn2cc(CNc3cc(Cl)ncn3)nc12] 1-(2-(((6-chloropyrimidin-4-yl)amino)methyl)-6- cyclopropylimidazo[1,2-a]pyridin-8-yl)ethan-1- one | 1-(2-(aminomethyl)-6- cyclopropylimidazo[1,2-a]pyridin-8-yl)ethan- 1-one ESI-MS [M + H]⁺: 342.2 |
| 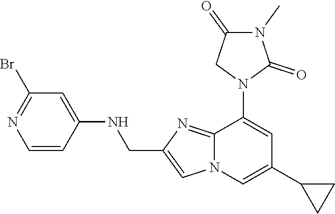 [SMILES: CN1C(=O)CN(c2cc(C3CC3)cn3cc(CNc4ccnc(Br)c4)nc23)C1=O] 1-(2-(((2-bromopyridin-4-yl)amino)methyl)-6- cyclopropylimidazo[1,2-a]pyridin-8-yl)-3- methylimidazolidine-2,4-dione | 1-(2-(aminomethyl)-6- cyclopropylimidazo[1,2-a]pyridin-8-yl)-3- methylimidazolidine-2,4-dione ESI-MS [M + H]⁺: 455.2 |
| 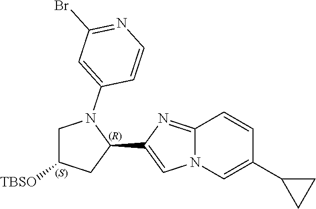 2-((2R,4S)-1-(2-bromopyridin-4-yl)-4-((tert- butyldimethylsilyl)oxy)pyrrolidin-2-yl)-6- cyclopropylimidazo[1,2-a]pyridine | 2-((2R,4S)-4-((tert- butyldimethylsilyl)oxy)pyrrolidin-2-yl)-6- cyclopropylimidazo[1,2-a]pyridine ESI-MS [M + H]⁺: 513.1 |
| 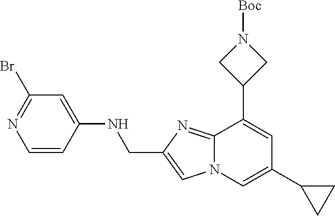 [SMILES: CC(C)(C)OC(=O)N1CC(c2cc(C3CC3)cn3cc(CNc4ccnc(Br)c4)nc23)C1] tert-butyl 3-(2-(((2-bromopyridin-4- yl)amino)methyl)-6-cyclopropylimidazo[1,2- a]pyridin-8-yl)azetidine-1-carboxylate | tert-butyl 3-(2-(aminomethyl)-6- cyclopropylimidazo[1,2-a]pyridin-8- yl)azetidine-1-carboxylate ESI-MS [M + H]⁺: 498.1 |
| 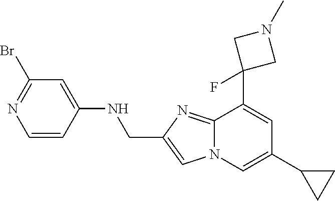 [SMILES: CN1CC(F)(c2cc(C3CC3)cn3cc(CNc4ccnc(Br)c4)nc23)C1] 2-bromo-N-((6-cyclopropyl-8-(3-fluoro-1- methylazetidin-3-yl)imidazo[1,2-a]pyridin-2- yl)methyl)pyridin-4-amine | (6-cyclopropyl-8-(3-fluoro-1- methylazetidin-3-yl)imidazo[1,2-a]pyridin- 2-yl)methanamine ESI-MS [M + H]⁺: 430.1 |
| 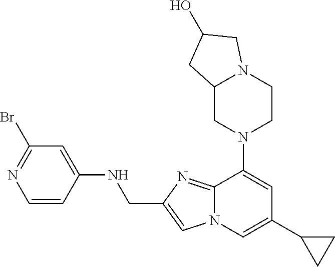 [SMILES: OC1CC2CN(c3cc(C4CC4)cn4cc(CNc5ccnc(Br)c5)nc34)CCN2C1] 2-(2-(((2-bromopyridin-4-yl)amino)methyl)-6- cyclopropylimidazo[1,2-a]pyridin-8- yl)octahydropyrrolo[1,2-a]pyrazin-7-ol | 2-(2-(aminomethyl)-6- cyclopropylimidazo[1,2-a]pyridin-8- yl)octahydropyrrolo[1,2-a]pyrazin-7-ol ESI-MS [M + H]⁺: 483.2 |
| 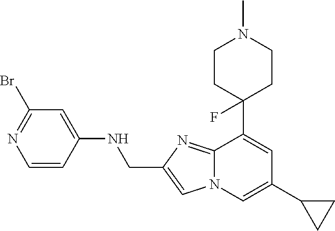 [SMILES: CN1CCC(F)(c2cc(C3CC3)cn3cc(CNc4ccnc(Br)c4)nc23)CC1] 2-bromo-N-((6-cyclopropyl-8-(4-fluoro-1- methylpiperidin-4-yl)imidazo[1,2-a]pyridin-2- yl)methyl)pyridin-4-amine | (6-cyclopropyl-8-(4-fluoro-1- methylpiperidin-4-yl)imidazo[1,2-a]pyridin- 2-yl)methanamine ESI-MS [M + H]⁺: 458.2 |
| 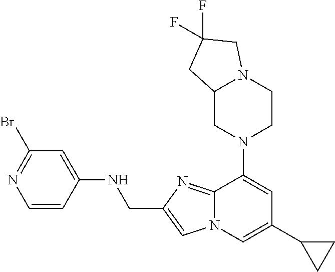 [SMILES: FC1(F)CC2CN(c3cc(C4CC4)cn4cc(CNc5ccnc(Br)c5)nc34)CCN2C1] 2-bromo-N-((6-cyclopropyl-8-(7,7- difluorohexahydropyrrolo[1,2-a]pyrazin-2(1H)- yl)imidazo[1,2-a]pyridin-2-yl)methyl)pyridin-4- amine | (6-cyclopropyl-8-(7,7- difluorohexahydropyrrolo[1,2-a]pyrazin- 2(1H)-yl)imidazo[1,2-a]pyridin-2- yl)methanamine ESI-MS [M + H]⁺: 503.1 |
| 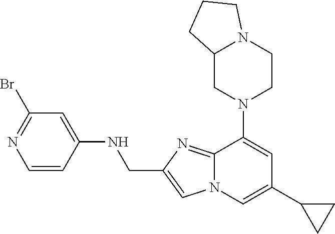 [SMILES: Brc1cc(NCc2cn3cc(C4CC4)cc(N4CCN5CCCC5C4)c3n2)ccn1] 2-bromo-N-((6-cyclopropyl-8- (hexahydropyrrolo[ 1,2-a]pyrazin-2(1H)- yl)imidazo[1,2-a]pyridin-2-yl)methyl)pyridin-4- amine | (6-cyclopropyl-8-(hexahydropyrrolo[1,2- a]pyrazin-2(1H)-yl)imidazo[1,2-a]pyridin-2- yl)methanamine ESI-MS [M + H]⁺: 467.1 |
| 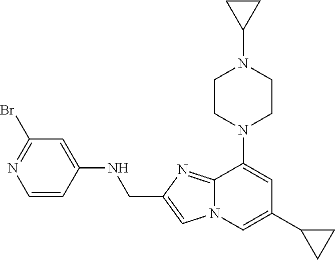 [SMILES: Brc1cc(NCc2cn3cc(C4CC4)cc(N4CCN(C5CC5)CC4)c3n2)ccn1] 2-bromo-N-((6-cyclopropyl-8-(4- cyclopropylpiperazin-1-yl)imidazo[1,2- a]pyridin-2-yl)methyl)pyridin-4-amine | (6-cyclopropyl-8-(4-cyclopropylpiperazin- 1-yl)imidazo[1,2-a]pyridin-2- yl)methanamine ESI-MS [M + H]⁺: 467.1 |
| 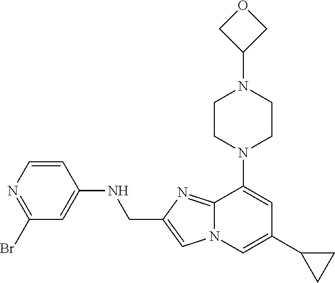 [SMILES: Brc1cc(NCc2cn3cc(C4CC4)cc(N4CCN(C5COC5)CC4)c3n2)ccn1] 2-bromo-N-((6-cyclopropyl-8-(4-(oxetan-3- yl)piperazin-1-yl)imidazo[1,2-a]pyridin-2- yl)methyl)pyridin-4-amine | (6-cyclopropyl-8-(4-(oxetan-3-yl)piperazin- 1-yl)imidazo[1,2-a]pyridin-2- yl)methanamine ESI-MS [M + H]⁺: 483.2 |
| 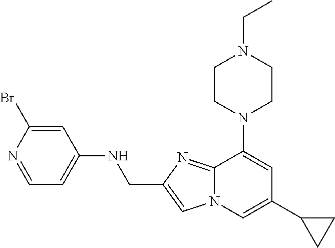 [SMILES: CCN1CCN(c2cc(C3CC3)cn3cc(CNc4ccnc(Br)c4)nc23)CC1] 2-bromo-N-((6-cyclopropyl-8-(4- ethylpiperazin-1-yl)imidazo[1,2-a]pyridin-2- yl)methyl)pyridin-4-amine | (6-cyclopropyl-8-(4-ethylpiperazin-1- yl)imidazo[1,2-a]pyridin-2-yl)methanamine ESI-MS [M + H]⁺: 455.3 |
| 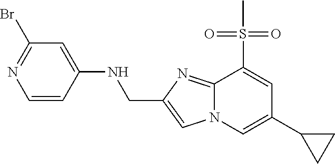 [SMILES: CS(=O)(=O)c1cc(C2CC2)cn2cc(CNc3ccnc(Br)c3)nc12] 2-bromo-N-((6-cyclopropyl-8- (methylsulfonyl)imidazo[1,2-a]pyridin-2- yl)methyl)pyridin-4-amine | (6-cyclopropyl-8- (methylsulfonyl)imidazo[1,2-a]pyridin-2- yl)methanamine ESI-MS [M + H]⁺: 421.2 |
| 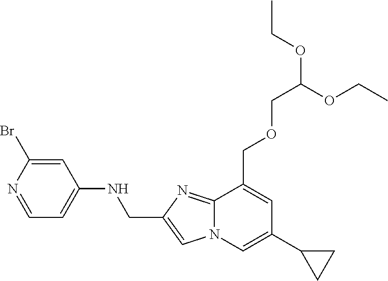 [SMILES: CCOC(COCc1cc(C2CC2)cn2cc(CNc3ccnc(Br)c3)nc12)OCC] 2-bromo-N-((6-cyclopropyl-8-((2,2- diethoxyethoxy)methyl)imidazo[1,2-a]pyridin- 2-yl)methyl)pyridin-4-amine | (6-cyclopropyl-8-((2,2- diethoxyethoxy)methyl)imidazo[1,2- a]pyridin-2-yl)methanamine ESI-MS [M + H]⁺: 489.1 |
| 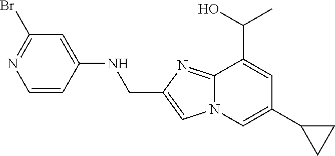 [SMILES: CC(O)c1cc(C2CC2)cn2cc(CNc3ccnc(Br)c3)nc12] 1-(2-(((2-bromopyridin-4-yl)amino)methyl)-6- cyclopropylimidazo[1,2-a]pyridin-8-yl)ethan-1- ol | 1-(2-(aminomethyl)-6- cyclopropylimidazo[1,2-a]pyridin-8- yl)ethan-1-ol ESI-MS [M + H]⁺: 387.1 |
| 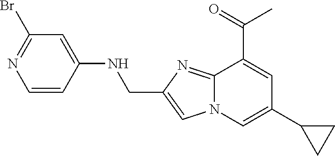 [SMILES: CC(=O)c1cc(C2CC2)cn2cc(CNc3ccnc(Br)c3)nc12] 1-(2-(((2-bromopyridin-4-yl)amino)methyl)-6- cyclopropylimidazo[1,2-a]pyridin-8-yl)ethan-1- one | 1-(2-(aminomethyl)-6- cyclopropylimidazo[1,2-a]pyridin-8- yl)ethan-1-one ESI-MS [M + H]⁺: 385.1 |
| 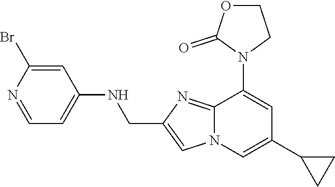 [SMILES: O=C1OCCN1c1cc(C2CC2)cn2cc(CNc3ccnc(Br)c3)nc12] 3-(2-(((2-bromopyridin-4-yl)amino)methyl)-6- cyclopropylimidazo[1,2-a]pyridin-8- yl)oxazolidin-2-one | 3-(2-(aminomethyl)-6- cyclopropylimidazo[1,2-a]pyridin-8- yl)oxazolidin-2-one ESI-MS [M + H]⁺: 428.2 |
| 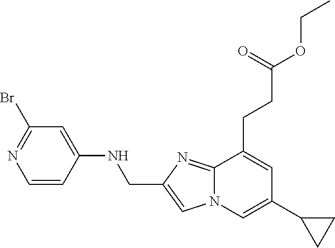 [SMILES: CCOC(=O)CCc1cc(C2CC2)cn2cc(CNc3ccnc(Br)c3)nc12] ethyl 3-(2-(((2-bromopyridin-4- yl)amino)methyl)-6-cyclopropylimidazo[1,2- a]pyridin-8-yl)propanoate | ethyl 3-(2-(aminomethyl)-6- cyclopropylimidazo[1,2-a]pyridin-8- yl)propanoate ESI-MS [M + H]⁺: 443.2 |
| 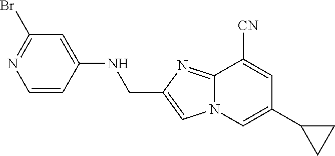 [SMILES: N#Cc1cc(C2CC2)cn2cc(CNc3ccnc(Br)c3)nc12] 2-(((2-bromopyridin-4-yl)amino)methyl)-6- cyclopropylimidazo[1,2-a]pyridine-8- carbonitrile | 2-(aminomethyl)-6-cyclopropylimidazo[1,2- a]pyridine-8-carbonitrile ESI-MS [M + H]⁺: 368.2 |
| 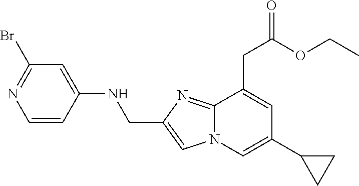 [SMILES: CCOC(=O)Cc1cc(C2CC2)cn2cc(CNc3ccnc(Br)c3)nc12] ethyl 2-(2-(((2-bromopyridin-4- yl)amino)methyl)-6-cyclopropylimidazo[1,2- a]pyridin-8-yl)acetate | ethyl 2-(2-(aminomethyl)-6- cyclopropylimidazo[1,2-a]pyridin-8- yl)acetate ESI-MS [M + H]⁺: 429.1 |
| 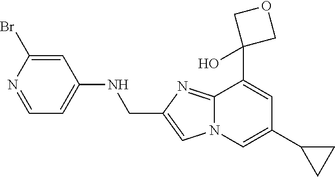 [SMILES: OC1(c2cc(C3CC3)cn3cc(CNc4ccnc(Br)c4)nc23)COC1] 3-(2-(((2-bromopyridin-4-yl)amino)methyl)-6- cyclopropylimidazo[1,2-a]pyridin-8-yl)oxetan- 3-ol | 3-(2-(aminomethyl)-6- cyclopropylimidazo[1,2-a]pyridin-8- yl)oxetan-3-ol ESI-MS [M + H]⁺: 415.1 |
| 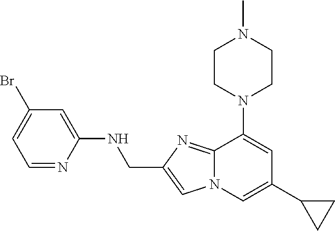 [SMILES: CN1CCN(c2cc(C3CC3)cn3cc(CNc4cc(Br)ccn4)nc23)CC1] 4-bromo-N-((6-cyclopropyl-8-(4- methylpiperazin-1-yl)imidazo[1,2-a]pyridin-2- yl)methyl)pyridin-2-amine | (6-cyclopropyl-8-(4-methylpiperazin-1- yl)imidazo[1,2-a]pyridin-2-yl)methanamine ESI-MS [M + H]⁺: 441.2 |
| 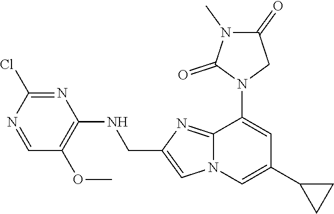 [SMILES: COc1cnc(Cl)nc1NCc1cn2cc(C3CC3)cc(N3CC(=O)N(C)C3=O)c2n1] 1-(2-(((2-chloro-5-methoxypyrimidin-4-yl) amino)methyl)-6-cyclopropylimidazo[1,2-a] pyridin-8-yl)-3-methylimidazolidine-2,4-dione | 1-(2-(aminomethyl)-6- cyclopropylimidazo[1,2-a]pyridin-8-yl)-3- methylimidazolidine-2,4-dione ESI-MS [M + H]⁺: 442.2 |
| 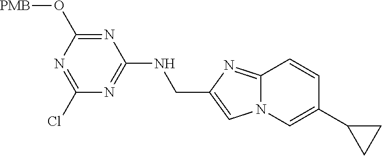 4-chloro-N-((6-cyclopropylimidazo[1,2- a]pyridin-2-yl)methyl)-6-((4- methoxybenzyl)oxy)-1,3,5-triazin-2-amine | (6-cyclopropylimidazo[1,2-a]pyridin-2- yl)methanamine ESI-MS [M + H]⁺: 437.2 |
| 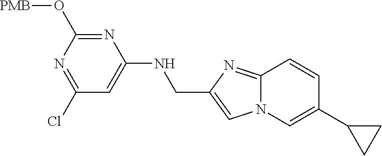 6-chloro-N-((6-cyclopropylimidazo[1,2- a]pyridin-2-yl)methyl)-2-((4- methoxybenzyl)oxy)pyrimidin-4-amine | (6-cyclopropylimidazo[1,2-a]pyridin-2- yl)methanamine ESI-MS [M + H]⁺: 436.2 |
| 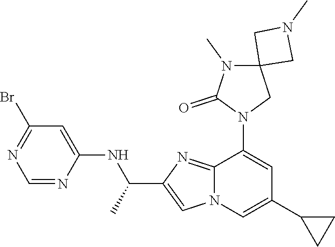 [SMILES: C[C@H](Nc1cc(Br)ncn1)c1cn2cc(C3CC3)cc(N3CC4(CN(C)C4)N(C)C3=O)c2n1] (R)-7-(2-(1-((6-bromopyrimidin-4- yl)amino)ethyl)-6-cyclopropylimidazo[1,2- a]pyridin-8-yl)-2,5-dimethyl-2,5,7- triazaspiro[3.4]octan-6-one | (S)-7-(2-(1-aminoethyl)-6- cyclopropylimidazo[1,2-a]pyridin-8-yl)-2,5- dimethyl-2,5,7-triazaspiro[3.4]octan-6-one ESI-MS [M + H]⁺: 511.1 |
| 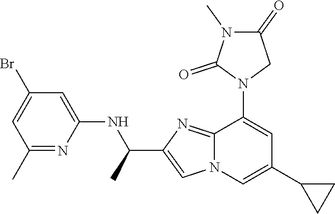 [SMILES: Cc1cc(Br)cc(N[C@H](C)c2cn3cc(C4CC4)cc(N4CC(=O)N(C)C4=O)c3n2)n1] (R)-1-(2-(1-((4-bromo-6-methylpyridin-2-yl) amino)ethyl)-6-cyclopropylimidazo[1,2-a] pyridin-8-yl)-3-methylimidazolidine-2,4-dione | (R)-1-(2-(1-aminoethyl)-6- cyclopropylimidazo[1,2-a]pyridin-8-yl)-3- methylimidazolidine-2,4-dione ESI-MS [M + H]+: 483.3 |
| 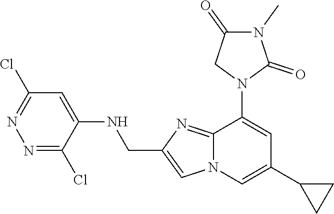 [SMILES: CN1C(=O)CN(c2cc(C3CC3)cn3cc(CNc4cc(Cl)nnc4Cl)nc23)C1=O] 1-(6-cyclopropyl-2-(((3,6-dichloropyridazin-4- yl)amino)methyl)imidazo[1,2-a]pyridin-8-yl)-3- methylimidazolidine-2,4-dione | 1-(2-(aminomethyl)-6- cyclopropylimidazo[1,2-a]pyridin-8-yl)-3- methylimidazolidine-2,4-dione ESI-MS [M + H]⁺: 446.2 |
| 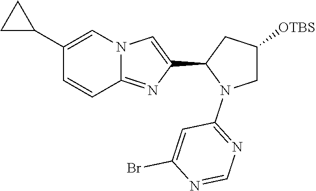 [SMILES: CC(C)(C)[Si](C)(C)O[C@H]1C[C@H](c2cn3cc(C4CC4)ccc3n2)N(c2cc(Br)ncn2)C1] 2-((2R,4S)-1-(6-bromopyrimidin-4-yl)-4-((tert- butyldimethylsilyl)oxy)pyrrolidin-2-yl)-6- cyclopropylimidazo[1,2-a]pyridine | 2-((2R,4S)-4-((tert- butyldimethylsilyl)oxy)pyrrolidin-2-yl)-6- cyclopropylimidazo[1,2-a]pyridine ESI-MS [M + H]⁺: 514.2 |
| 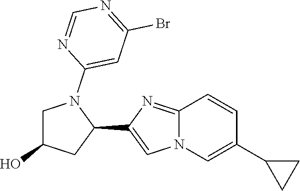 [SMILES: O[C@@H]1C[C@H](c2cn3cc(C4CC4)ccc3n2)N(c2cc(Br)ncn2)C1] (3R,5R)-1-(6-bromopyrimidin-4-yl)-5-(6- cyclopropylimidazo[1,2-a]pyridin-2- yl)pyrrolidin-3-ol | (3R,5R)-5-(6-cyclopropylimidazo[1,2- a]pyridin-2-yl)pyrrolidin-3-ol ESI-MS [M + H]⁺: 400.2 |
| 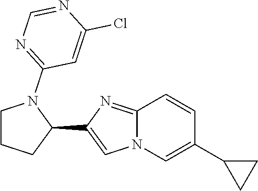 [SMILES: Clc1cc(N2CCC[C@@H]2c2cn3cc(C4CC4)ccc3n2)ncn1] (R)-2-(1-(6-chloropyrimidin-4-yl)pyrrolidin-2- yl)-6-cyclopropylimidazo[1,2-a]pyridine | (R)-6-cyclopropyl-2-(pyrrolidin-2- yl)imidazo[1,2-a]pyridine ESI-MS [M + H]⁺: 340.2 |
| 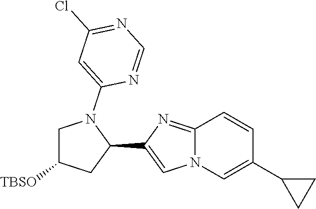 [SMILES: CC(C)(C)[Si](C)(C)O[C@H]1C[C@H](c2cn3cc(C4CC4)ccc3n2)N(c2cc(Cl)ncn2)C1] 2-((2R,4S)-4-((tert-butyldimethylsilyl)oxy)-1- (6-chloropyrimidin-4-yl)pyrrolidin-2-yl)-6- cyclopropylimidazo[1,2-a]pyridine | 2-((2R,4S)-4-((tert- butyldimethylsilyl)oxy)pyrrolidin-2-yl)-6- cyclopropylimidazo[1,2-a]pyridine ESI-MS [M + H]⁺: 470.2 |
| 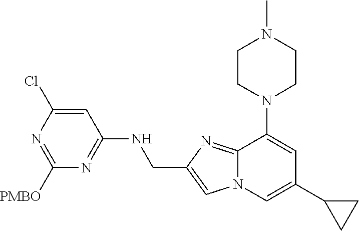 6-chloro-N-((6-cyclopropyl-8-(4-methyl- piperazin-1-yl)imidazo[1,2-a]pyridin-2-yl)methyl)- 2-((4-methoxybenzyl)oxy)pyrimidin-4-amine | (6-cyclopropyl-8-(4-methylpiperazin-1- yl)imidazo[1,2-a]pyridin-2-yl)methanamine ESI-MS [M + H]⁺: 534.3 |
| 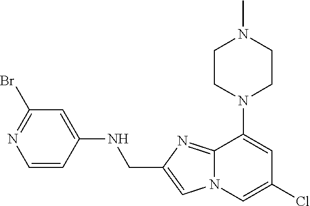 [SMILES: CN1CCN(c2cc(Cl)cn3cc(CNc4ccnc(Br)c4)nc23)CC1] 2-bromo-N-((6-chloro-8-(4-methylpiperazin-1- yl)imidazo[1,2-a]pyridin-2-yl)methyl)pyridin-4- amine | (6-chloro-8-(4-methylpiperazin-1- yl)imidazo[1,2-a]pyridin-2-yl)methanamine ESI-MS [M + H]⁺: 435.2 |
| 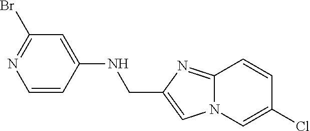 [SMILES: Clc1ccc2nc(CNc3ccnc(Br)c3)cn2c1] 2-bromo-N-((6-chloroimidazo[1,2-a]pyridin-2- yl)methyl)pyridin-4-amine | (6-chloroimidazo[1,2-a]pyridin-2- yl)methanamine ESI-MS [M + H]⁺: 337.2 |
| 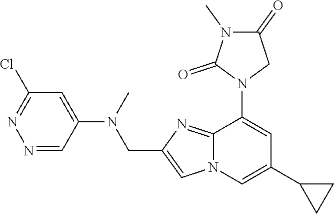 [SMILES: CN1C(=O)CN(c2cc(C3CC3)cn3cc(CN(C)c4cnnc(Cl)c4)nc23)C1=O] 1-(2-(((6-chloropyridazin-4-yl)(methyl)amino) methyl)-6-cyclopropylimidazo[1,2-a]pyridin-8- yl)-3-methylimidazolidine-2,4-dione | 1-(6-cyclopropyl-2- ((methylamino)methyl)imidazo[1,2- a]pyridin-8-yl)-3-methylimidazolidine-2,4- dione ESI-MS [M + H]⁺: 426.2 |
| 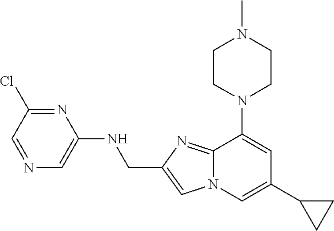 [SMILES: CN1CCN(c2cc(C3CC3)cn3cc(CNc4cncc(Cl)n4)nc23)CC1] 6-chloro-N-((6-cyclopropyl-8-(4- methylpiperazin-1-yl)imidazo[1,2-a]pyridin-2- yl)methyl)pyrazin-2-amine | (6-cyclopropyl-8-(4-methylpiperazin-1- yl)imidazo[1,2-a]pyridin-2-yl)methanamine ESI-MS [M + H]⁺: 398.2 |

### Example 144

### Synthesis of (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide (I-144)

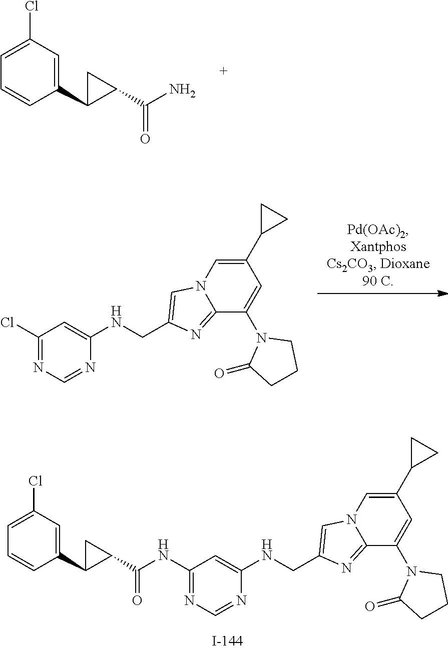 [SMILES: NC(=O)[C@H]1C[C@@H]1c1cccc(Cl)c1 ; O=C1CCCN1c1cc(C2CC2)cn2cc(CNc3cc(Cl)ncn3)nc12 ; "Pd(OAc)2,XantphosCs2CO3, Dioxane90 C." ; O=C(Nc1cc(NCc2cn3cc(C4CC4)cc(N4CCCC4=O)c3n2)ncn1)[C@H]1C[C@@H]1c1cccc(Cl)c1]

A suspension of 1-(2-(((6-chloropyrimidin-4-yl)amino)methyl)-6-cyclopropylimidazo[1,2-a]pyridin-8-yl)pyrrolidin-2-one (0.16 g, 0.70 mmol), (1S,2S)-2-(3-chlorophenyl)cyclopropane-1-carboxamide (0.16 g, 0.83 mmol), Pd(OAc)₂ (0.037 g, 0.17 mmol), Xantphos (0.19 g, 0.34 mmol), and Cs₂CO₃ (0.54 g, 1.66 mmol) in 1,4-dioxane (10 mL) was degassed with N₂ for 10 min, then heated to 80° C. for 3 h. The mixture was cooled to room temperature, diluted with water (50 mL), then extracted with EtOAc (3×20 mL), then extracted with 10% MeOH in DCM (3×20 mL). The combined organics were dried over MgSO₄, then concentrated in vacuo. The residue was purified by silica gel chromatography, eluting with a gradient of DCM—12% (7N NH₃ in MeOH) in DCM. This crude product was triturated with diethyl ether to give the desired product (0.15 g, 38%). ESI-MS (M+H)+: 542.4, ¹H NMR (400 MHz, DMSO) δ 10.65 (s, 1H), 8.29 (s, 1H), 8.25 (s, 1H), 7.93 (s, 1H), 7.70 (s, 1H), 7.40-7.29 (m, 4H), 7.23-7.16 (m, 2H), 4.62-4.61 (m, 2H), 4.20 (dd, J=6.9, 6.9 Hz, 2H), 2.56 (dd, J=1.6, 1.6 Hz, 9H), 2.54-2.50 (m, 2H), 2.49-2.40 (m, 2H), 2.22-2.13 (m, 2H), 2.01-1.93 (m, 1H), 1.56-1.42 (m, 2H), 1.01-0.94 (m, 2H), 0.72-0.66 (m, 2H).

Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the compounds in Table 26 were prepared using a cyclopropyl carboxamide and an appropriate coupling partner, then purified by preparative LCMS.

TABLE 26

| | | Coupling Partner/ |
|---|---|---|
| Example | Compound | Analytical Data |
| Example 145 | 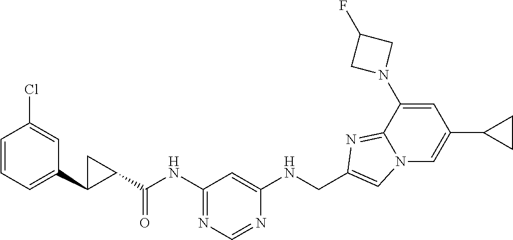 [SMILES: O=C(Nc1cc(NCc2cn3cc(C4CC4)cc(N4CC(F)C4)c3n2)ncn1)[C@H]1C[C@@H]1c1cccc(Cl)c1] (1S,2S)-2-(3-chlorophenyl)-N-(6-(((6- cyclopropyl-8-(3-fluoroazetidin-1- yl)imidazo[1,2-a]pyridin-2- yl)methyl)amino)pyrimidin-4- yl)cyclopropane-1-carboxamide (I- 145) | 6-chloro-N-((6-cyclopropyl-8-(3- fluoroazetidin-1-yl)imidazo[1,2- a]pyridin-2-yl)methyl)pyrimidin-4- amine ESI-MS (M + H)+: 532.4, ¹H NMR (400 MHz, DMSO) δ 10.59 (s, 1H), 8.19 (s, 1H), 7.80 (br s, 1H), 7.72 (d, J = 0.9 Hz, 1H), 7.50 (s, 1H), 7.34-7.25 (m, 4H), 7.17-7.12 (m, 1H), 5.73-5.71 (m, 1H), 5.57-5.37 (m, 1H), 4.52-4.39 (m, 4H), 4.19-4.07 (m, 2H), 2.44-2.32 (m, 2H), 1.85-1.77 (m, 1H), 1.50-1.36 (m, 2H), 0.88-0.81 (m, 2H), 0.66-0.60 (m, 2H). |
| Example 146 | 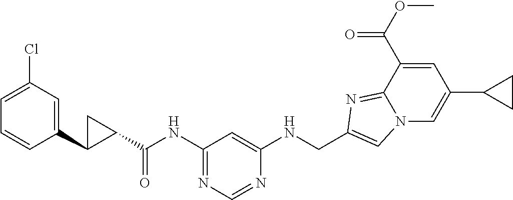 [SMILES: COC(=O)c1cc(C2CC2)cn2cc(CNc3cc(NC(=O)[C@H]4C[C@@H]4c4cccc(Cl)c4)ncn3)nc12] methyl 2-(((6-((1S,2S)-2-(3- chlorophenyl)cyclopropane-1- carboxamido)pyrimidin-4- yl)amino)methyl)-6- cyclopropylimidazo[1,2-a]pyridine-8- carboxylate (I-146) | methyl 2-(((6-chloropyrimidin-4- yl)amino)methyl)-6- cyclopropylimidazo[1,2-a]pyridine-8- carboxylate ESI-MS (M + H)+: 517.3, ¹H NMR (400 MHz, DMSO) δ 10.62 (s, 1H), 8.57 (d, J = 1.3 Hz, 1H), 8.21 (s, 1H), 7.96 (br s, 1H), 7.73 (s, 1H), 7.60 (d, J = 1.4 Hz, 1H), 7.36-7.26 (m, 4H), 7.17 (d, J = 8.2 Hz, 1H), 4.62 (br s, 2H), 3.91 (s, 3H), 2.41-2.40 (m, 2H), 2.01-2.01 (m, 1H), 1.49-1.48 (m, 1H), 1.42-1.42 (m, 1H), 0.97-0.96 (m, 2H), 0.71-0.71 (m, 2H). |
| Example 147 | 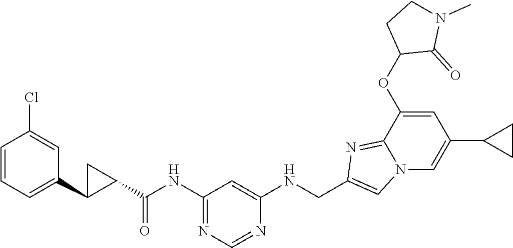 [SMILES: CN1CCC(Oc2cc(C3CC3)cn3cc(CNc4cc(NC(=O)[C@H]5C[C@@H]5c5cccc(Cl)c5)ncn4)nc23)C1=O] (1S,2S)-2-(3-chlorophenyl)-N-(6-(((6- cyclopropyl-8-((1-methyl-2- oxopyrrolidin-3-yl)oxy)imidazo[1,2- a]pyridin-2- yl)methyl)amino)pyrimidin-4- yl)cyclopropane-1-carboxamide (I- 147) | 3-((2-(((6-chloropyrimidin-4- yl)amino)methyl)-6- cyclopropylimidazo[1,2-a]pyridin-8- yl)oxy)-1-methylpyrrolidin-2-one ESI-MS (M + H)+: 572.4, ¹H NMR (400 MHz, DMSO) δ 10.59 (s, 1H), 8.21 (s, 1H), 7.98 (s, 1H), 7.89 (s, 1H), 7.61 (s, 1H), 7.35-7.30 (m, 1H), 7.27 (dd, J = 1.8, 3.6 Hz, 3H), 7.16 (d, J = 7.5 Hz, 1H), 6.54 (s, 1H), 5.35 (dd, J = 7.3, 7.3 Hz, 1H), 4.55-4.55 (m, 2H), 3.46 (ddd, J = 2.9, 8.3, 10.3 Hz, 1H), 3.40-3.35 (m, 1H), 2.83 (s, 3H), 2.63-2.54 (m, 1H), 2.45-2.35 (m, 2H), 2.06-1.85 (m, 2H), 1.51-1.38 (m, 2H), 0.93-0.89 (m, 2H), 0.70-0.67 (m, 2H). |
| Example 148 | 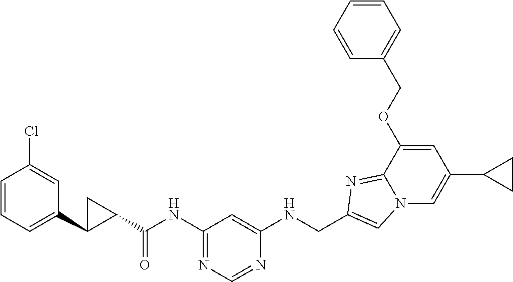 [SMILES: O=C(Nc1cc(NCc2cn3cc(C4CC4)cc(OCc4ccccc4)c3n2)ncn1)[C@H]1C[C@@H]1c1cccc(Cl)c1] (1S,2S)-N-(6-(((8-(benzyloxy)-6- cyclopropylimidazo[1,2-a]pyridin-2- yl)methyl)amino)pyrimidin-4-yl)-2-(3- chlorophenyl)cyclopropane-1- carboxamide (I-148) | N-((8-(benzyloxy)-6- cyclopropylimidazo[1,2-a]pyridin-2- yl)methyl)-6-chloropyrimidin-4-amine ESI-MS (M + H)+: 565.6, ¹H NMR (400 MHz, DMSO) δ 10.59 (s, 1H), 8.21- 8.19 (m, 1H), 7.96-7.94 (m, 1H), 7.88 (br s, 1H), 7.60 (s, 1H), 7.53-7.50 (m, 2H), 7.46-7.30 (m, 4H), 7.28-7.26 (m, 3H), 7.18-7.14 (m, 1H), 6.46 (d, J = 1.1 Hz, 1H), 5.27 (s, 2H), 4.54 (br s, 2H), 2.45-2.35 (m, 2H), 1.92-1.85 (m, 1H), 1.51-1.38 (m, 2H), 0.93-0.87 (m, 2H), 0.71-0.66 (m, 2H). |
| Example 149 | 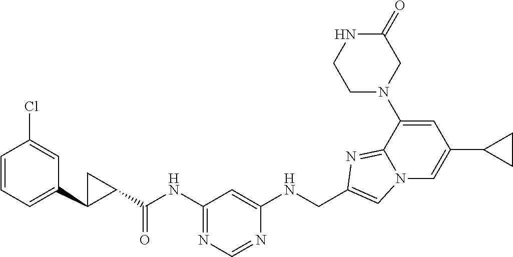 [SMILES: O=C1CN(c2cc(C3CC3)cn3cc(CNc4cc(NC(=O)[C@H]5C[C@@H]5c5cccc(Cl)c5)ncn4)nc23)CCN1] (1S,2S)-2-(3-chlorophenyl)-N-(6-(((6- cyclopropyl-8-(3-oxopiperazin-1- yl)imidazo[1,2-a]pyridin-2- yl)methyl)amino)pyrimidin-4- yl)cyclopropane-1-carboxamide (I- 149) | 4-(2-(((6-chloropyrimidin-4- yl)amino)methyl)-6- cyclopropylimidazo[1,2-a]pyridin-8- yl)piperazin-2-one ESI-MS (M + H)+: 557.4, ¹H NMR (400 MHz, DMSO) δ 10.58 (s, 1H), 8.19 (s, 1H), 8.02-7.99 (m, 1H), 7.90-7.85 (m, 2H), 7.54 (s, 1H), 7.34-7.23 (m, 4H), 7.17-7.13 (m, 1H), 6.17 (s, 1H), 4.54 (br s, 2H), 4.00 (s, 2H), 3.83 (t, J = 4.9 Hz, 2H), 2.43-2.32 (m, 2H), 1.91-1.82 (m, 1H), 1.50-1.36 (m, 2H), 0.90-0.84 (m, 2H), 0.70-0.64 (m, 2H). One piperazinone CH2 signal obscured by H2O signal. |
| Example 150 | 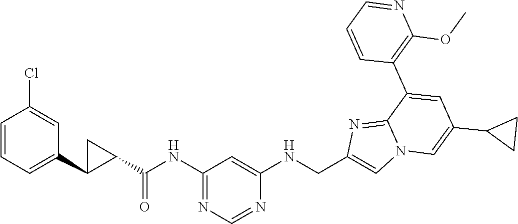 [SMILES: COc1ncccc1-c1cc(C2CC2)cn2cc(CNc3cc(NC(=O)[C@H]4C[C@@H]4c4cccc(Cl)c4)ncn3)nc12] (1S,2S)-2-(3-chlorophenyl)-N-(6-(((6- cyclopropyl-8-(2-methoxypyridin-3- yl)imidazo[1,2-a]pyridin-2- yl)methyl)amino)pyrimidin-4- yl)cyclopropane-1-carboxamide (I- 150) | 6-chloro-N-((6-cyclopropyl-8-(2- methoxypyridin-3-yl)imidazo[1,2- a]pyridin-2-yl)methyl)pyrimidin-4- amine ESI-MS (M + H)+: 566.4, ¹H NMR (400 MHz, DMSO) δ 10.60 (s, 1H), 8.33 (d, J = 1.5 Hz, 1H), 8.24 (dd, J = 1.9, 5.0 Hz, 1H), 8.20 (s, 1H), 8.05 (d, J = 6.1 Hz, 1H), 7.88 (s, 1H), 7.68 (s, 1H), 7.36- 7.31 (m, 1H), 7.27 (d, J = 5.3 Hz, 3H), 7.18-7.11 (m, 2H), 7.09 (s, 1H), 4.54- 4.53 (m, 2H), 3.85 (s, 3H), 2.44-2.34 (m, 3H), 1.99-1.92 (m, 1H), 1.50-1.39 (m, 2H), 0.97-0.92 (m, 2H), 0.73-0.68 (m, 2H). |
| Example 151 | 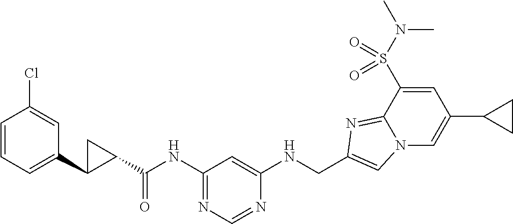 [SMILES: CN(C)S(=O)(=O)c1cc(C2CC2)cn2cc(CNc3cc(NC(=O)[C@H]4C[C@@H]4c4cccc(Cl)c4)ncn3)nc12] (1S,2S)-2-(3-chlorophenyl)-N-(6-(((6- cyclopropyl-8-(N,N- dimethylsulfamoyl)imidazo[1,2- a]pyridin-2- yl)methyl)amino)pyrimidin-4- yl)cyclopropane-1-carboxamide (I- 151) | 2-(((6-chloropyrimidin-4- yl)amino)methyl)-6-cyclopropyl-N,N- dimethylimidazo[1,2-a]pyridine-8- sulfonamide ESI-MS (M + H)+: 566.3, ¹H NMR (400 MHz, DMSO) δ 10.64 (s, 1H), 8.57- 8.55 (m, 1H), 8.21-8.19 (m, 1H), 7.98 (br s, 1H), 7.77 (s, 1H), 7.51-7.50 (m, 1H), 7.35-7.24 (m, 4H), 7.17-7.13 (m, 1H), 4.62 (br s, 2H), 2.84 (s, 6H), 2.42- 2.32 (m, 2H), 2.08-2.00 (m, 1H), 1.48- 1.38 (m, 2H), 0.99-0.93 (m, 2H), 0.73- 0.68 (m, 2H). |
| Example 152 | 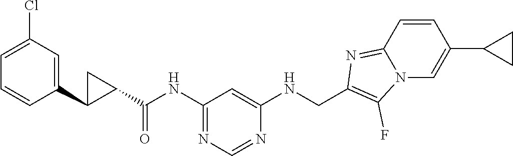 [SMILES: O=C(Nc1cc(NCc2nc3ccc(C4CC4)cn3c2F)ncn1)[C@H]1C[C@@H]1c1cccc(Cl)c1] (1S,2S)-2-(3-chlorophenyl)-N-(6-(((6- cyclopropyl-3-fluoroimidazo[1,2- a]pyridin-2- yl)methyl)amino)pyrimidin-4- yl)cyclopropane-1-carboxamide (I- 152) | 6-chloro-N-((6-cyclopropyl-3- fluoroimidazo[1,2-a]pyridin-2- yl)methyl)pyrimidin-4-amine ESI-MS (M + H)+: 477.3, ¹H NMR (400 MHz, DMSO) δ 10.63 (s, 1H), 8.25 (s, 1H), 8.08 (s, 1H), 7.94-7.88 (m, 1H), 7.40-7.29 (m, 5H), 7.20 (d, J = 7.6 Hz, 1H), 7.00 (dd, J = 1.7, 9.4 Hz, 1H), 4.63 (s, 2H), 2.47-2.38 (m, 2H), 2.08-2.01 (m, 1H), 1.55-1.42 (m, 2H), 1.01-0.95 (m, 2H), 0.81-0.76 (m, 2H). |
| Example 153 | 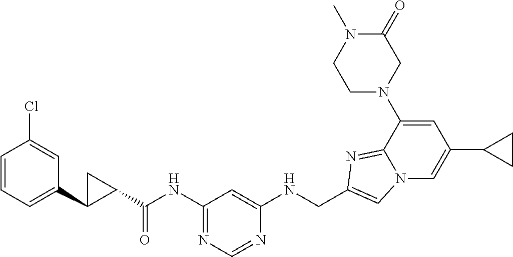 [SMILES: CN1CCN(c2cc(C3CC3)cn3cc(CNc4cc(NC(=O)[C@H]5C[C@@H]5c5cccc(Cl)c5)ncn4)nc23)CC1=O] (1S,2S)-2-(3-chlorophenyl)-N-(6-(((6- cyclopropyl-8-(4-methyl-3- oxopiperazin-1-yl)imidazo[1,2- a]pyridin-2- yl)methyl)amino)pyrimidin-4- yl)cyclopropane-1-carboxamide (I- 153) | 4-(2-(((6-chloropyrimidin-4- yl)amino)methyl)-6- cyclopropylimidazo[1,2-a]pyridin-8- yl)-1-methylpiperazin-2-one ESI-MS (M + H)+: 571.4, ¹H NMR (400 MHz, DMSO) δ 10.59 (s, 1H), 8.19 (s, 1H), 7.91-7.85 (m, 2H), 7.55 (s, 1H), 7.34-7.24 (m, 4H), 7.17-7.13 (m, 1H), 6.18 (d, J = 1.1 Hz, 1H), 4.55 (br s, 2H), 4.07 (s, 2H), 3.89 (dd, J = 5.1, 5.1 Hz, 2H), 3.45 (dd, J = 5.3, 5.3 Hz, 2H), 2.91- 2.90 (m, 3H), 2.43-2.32 (m, 2H), 1.90- 1.82 (m, 1H), 1.50-1.37 (m, 2H), 0.90- 0.84 (m, 2H), 0.70-0.65 (m, 2H). |
| Example 154 | 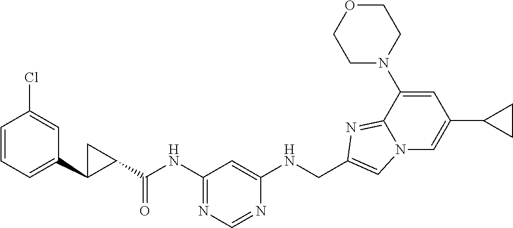 [SMILES: O=C(Nc1cc(NCc2cn3cc(C4CC4)cc(N4CCOCC4)c3n2)ncn1)[C@H]1C[C@@H]1c1cccc(Cl)c1] (1S,2S)-2-(3-chlorophenyl)-N-(6-(((6- cyclopropyl-8-morpholinoimidazo[1,2- a]pyridin-2- yl)methyl)amino)pyrimidin-4- yl)cyclopropane-1-carboxamide (I- 154) | 6-chloro-N-((6-cyclopropyl-8- morpholinoimidazo[1,2-a]pyridin-2- yl)methyl)pyrimidin-4-amine ESI-MS (M + H)+: 544, ¹H NMR (400 MHz, DMSO) δ 10.63 (s, 1H), 8.24 (s, 1H), 7.95-7.88 (m, 2H), 7.58 (s, 1H), 7.38-7.29 (m, 4H), 7.20 (d, J = 7.1 Hz, 1H), 6.21 (s, 1H), 4.58 (s, 2H), 3.87- 3.84 (m, 4H), 3.55-3.49 (m, 4H), 2.47- 2.43 (m, 2H), 1.93-1.87 (m, 1H), 1.54- 1.44 (m, 2H), 0.94-0.90 (m, 2H), 0.74- 0.68 (m, 2H). |
| Example 155 | 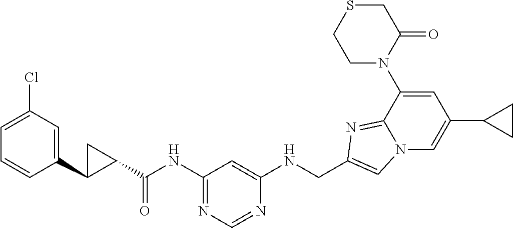 [SMILES: O=C(Nc1cc(NCc2cn3cc(C4CC4)cc(N4CCSCC4=O)c3n2)ncn1)[C@H]1C[C@@H]1c1cccc(Cl)c1] (1S,2S)-2-(3-chlorophenyl)-N-(6-(((6- cyclopropyl-8-(3- oxothiomorpholino)imidazo[1,2- a]pyridin-2- yl)methyl)amino)pyrimidin-4- yl)cyclopropane-1-carboxamide (I- 155) | 4-(2-(((6-chloropyrimidin-4- yl)amino)methyl)-6- cyclopropylimidazo[1,2-a]pyridin-8- yl)thiomorpholin-3-one ESI-MS (M + H)+: 574.5, ¹H NMR (400 MHz, DMSO) δ 10.59 (s, 1H), 8.27 (d, J = 1.0 Hz, 1H), 8.19 (s, 1H), 7.90 (br s, 1H), 7.66 (s, 1H), 7.34-7.24 (m, 4H), 7.15 (d, J = 7.8 Hz, 1H), 6.95 (d, J = 1.5 Hz, 1H), 4.56 (br s, 2H), 4.01 (m 2H), 3.46 (s, 2H), 3.16 (m 2H), 2.43-2.34 (m, 2H), 1.96-1.88 (m, 1H), 1.50-1.44 (m, 1H), 1.43-1.37 (m, 1H), 0.95-0.89 (m, 2H), 0.68-0.63 (m, 2H). |
| Example 156 | 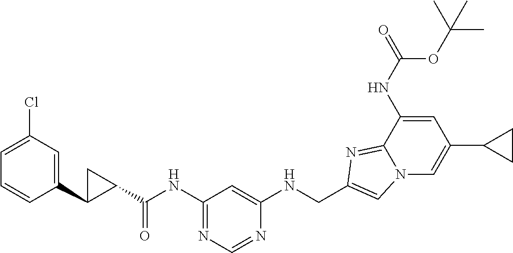 [SMILES: CC(C)(C)OC(=O)Nc1cc(C2CC2)cn2cc(CNc3cc(NC(=O)[C@H]4C[C@@H]4c4cccc(Cl)c4)ncn3)nc12] tert-butyl (2-(((6-((15,2S)-2-(3- chlorophenyl)cyclopropane-1- carboxamido)pyrimidin-4- yl)amino)methyl)-6- cyclopropylimidazo[1,2-a]pyridin-8- yl)carbamate (I-156) | tert-butyl (2-(((6-chloropyrimidin-4- yl)amino)methyl)-6- cyclopropylimidazo[1,2-a]pyridin-8- yl)carbamate ESI-MS (M + H)+: 574.4, ¹H NMR (400 MHz, DMSO) δ 10.65 (s, 1H), 8.29 (s, 1H), 8.25 (s, 1H), 8.04 (s, 1H), 7.89 (s, 1H), 7.68 (s, 1H), 7.40-7.18 (m, 7H), 4.62 (br s, 2H), 2.47-2.40 (m, 2H), 2.00- 1.89 (m, 1H), 1.55 (s, 9H), 1.48-1.36 (m, 2H), 0.99-0.93 (m, 2H), 0.70-0.65 (m, 2H). |
| Example 157 | 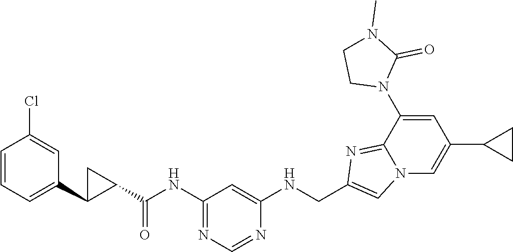 [SMILES: CN1CCN(c2cc(C3CC3)cn3cc(CNc4cc(NC(=O)[C@H]5C[C@@H]5c5cccc(Cl)c5)ncn4)nc23)C1=O] (1S,2S)-2-(3-chlorophenyl)-N-(6-(((6- cyclopropyl-8-(3-methyl-2- oxoimidazolidin-1-yl)imidazo[1,2- a]pyridin-2- yl)methyl)amino)pyrimidin-4- yl)cyclopropane-1-carboxamide (I- 157) | 1-(2-(((6-chloropyrimidin-4- yl)amino)methyl)-6- cyclopropylimidazo[1,2-a]pyridin-8- yl)-3-methylimidazolidin-2-one ESI-MS (M + H)+: 557.4, ¹H NMR (400 MHz, DMSO) δ 10.60 (s, 1H), 8.19 (s, 1H), 8.13-8.11 (m, 1H), 7.85 (br s, 1H), 7.62 (s, 1H), 7.34-7.21 (m, 5H), 7.17- 7.13 (m, 1H), 4.55 (br s, 2H), 4.32-4.25 (m, 2H), 3.49-3.44 (m, 2H), 2.78 (s, 3H), 2.44-2.34 (m, 2H), 1.92-1.84 (m, 1H), 1.50-1.37 (m, 2H), 0.93-0.87 (m, 2H), 0.64-0.58 (m, 2H). |
| Example 158 | 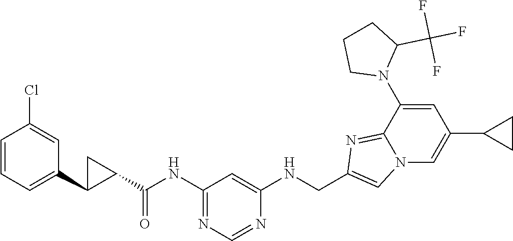 [SMILES: O=C(Nc1cc(NCc2cn3cc(C4CC4)cc(N4CCCC4C(F)(F)F)c3n2)ncn1)[C@H]1C[C@@H]1c1cccc(Cl)c1] (1S,2S)-2-(3-chlorophenyl)-N-(6-(((6- cyclopropyl-8-(2- (trifluoromethyl)pyrrolidin-1- yl)imidazo[1,2-a]pyridin-2- yl)methyl)amino)pyrimidin-4- yl)cyclopropane-1-carboxamide (I- 158) | 6-chloro-N-((6-cyclopropyl-8-(2- (trifluoromethyl)pyrrolidin-1- yl)imidazo[1,2-a]pyridin-2- yl)methyl)pyrimidin-4-amine ESI-MS (M + H)+: 596.3, ¹H NMR (400 MHz, DMSO) δ 10.59 (s, 1H), 8.18 (s, 1H), 7.79-7.77 (m, 2H), 7.52 (s, 1H), 7.33-7.29 (m, 2H), 7.28-7.24 (m, 2H), 7.15 (d, J = 7.6 Hz, 1H), 6.32-6.23 (m, 1H), 6.08 (s, 1H), 4.53-4.53 (m, 2H), 3.91-3.83 (m, 1H), 3.41-3.34 (m, 1H), 2.42-2.34 (m, 2H), 2.28-2.18 (m, 1H), 2.11-2.00 (m, 3H), 1.87-1.79 (m, 1H), 1.50-1.37 (m, 2H), 0.88-0.83 (m, 2H), 0.70-0.60 (m, 2H). |
| Example 159 | 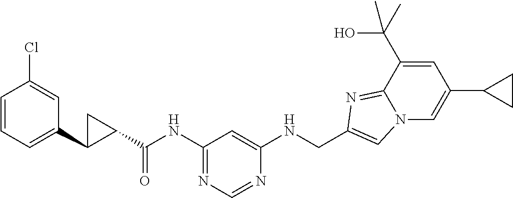 [SMILES: CC(C)(O)c1cc(C2CC2)cn2cc(CNc3cc(NC(=O)[C@H]4C[C@@H]4c4cccc(Cl)c4)ncn3)nc12] (1S,2S)-2-(3-chlorophenyl)-N-(6-(((6- cyclopropyl-8-(2-hydroxypropan-2- yl)imidazo[1,2-a]pyridin-2- yl)methyl)amino)pyrimidin-4- yl)cyclopropane-1-carboxamide (I- 159) | 2-(2-(((6-chloropyrimidin-4- yl)amino)methyl)-6- cyclopropylimidazo[1,2-a]pyridin-8- yl)propan-2-ol ESI-MS (M + H)+: 517.4, ¹H NMR (400 MHz, DMSO) δ 10.63 (s, 1H), 8.19 (d, J = 9.9 Hz, 2H), 7.89-7.89 (m, 1H), 7.58 (s, 1H), 7.36-7.30 (m, 2H), 7.28 (d, J = 6.4 Hz, 2H), 7.16 (d, J = 7.8 Hz, 1H), 7.06 (d, J = 1.5 Hz, 1H), 5.53 (s, 1H), 4.59-4.59 (m, 2H), 2.45-2.34 (m, 2H), 1.96-1.88 (m, 1H), 1.66 (s, 6H), 1.50- 1.39 (m, 2H), 0.95-0.89 (m, 2H), 0.69- 0.63 (m, 2H). |
| Example 160 | 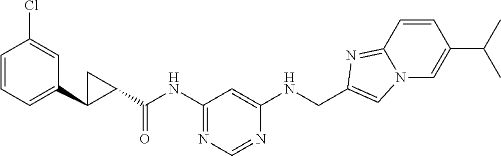 [SMILES: CC(C)c1ccc2nc(CNc3cc(NC(=O)[C@H]4C[C@@H]4c4cccc(Cl)c4)ncn3)cn2c1] (1S,2S)-2-(3-chlorophenyl)-N-(6-(((6- isopropylimidazo[1,2-a]pyridin-2- yl)methyl)amino)pyrimidin-4- yl)cyclopropane-1-carboxamide (I- 160) | 6-chloro-N-((6-isopropylimidazo[1,2- a]pyridin-2-yl)methyl)pyrimidin-4- amine ESI-MS (M + H)+: 461.3, ¹H NMR (400 MHz, DMSO) δ 10.58 (s, 1H), 8.32- 8.30 (m, 1H), 8.20-8.18 (m, 1H), 7.85 (br s, 1H), 7.64 (s, 1H), 7.40 (d, J = 9.3 Hz, 1H), 7.35-7.23 (m, 4H), 7.19-7.13 (m, 2H), 4.56 (br s, 2H), 2.93-2.82 (m, 1H), 2.44-2.34 (m, 2H), 1.50-1.37 (m, 2H), 1.22 (d, J = 6.8 Hz, 6H). |
| Example 161 | 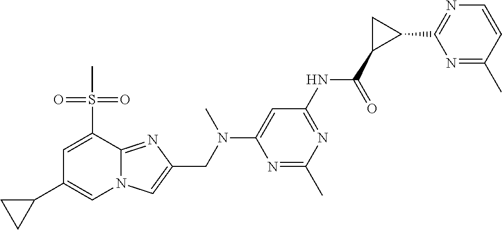 [SMILES: Cc1ccnc([C@H]2C[C@@H]2C(=O)Nc2cc(N(C)Cc3cn4cc(C5CC5)cc(S(C)(=O)=O)c4n3)nc(C)n2)n1] (1S,2S)-N-(6-(((6-cyclopropyl-8- (methylsulfonyl)imidazo[1,2-a]pyridin- 2-yl)methyl)(methyl)amino)-2- methylpyrimidin-4-yl)-2-(4- methylpyrimidin-2-yl)cyclopropane-1- carboxamide (I-161) | 6-chloro-N-((6-cyclopropyl-8- (methylsulfonyl)imidazo[1,2-a]pyridin- 2-yl)methyl)-N,2-dimethylpyrimidin-4- amine ESI-MS (M + H)+: 547.1, ¹H NMR (400 MHz, DMSO) δ 10.80 (s, 1H), 8.60 (d, J = 1.1 Hz, 1H), 8.52 (d, J = 5.1 Hz, 1H), 7.76 (s, 1H), 7.58 (d, J = 1.6 Hz, 1H), 7.28 (s, 1H), 7.20 (d, J = 5.1 Hz, 1H), 4.90 (s, 2H), 3.54 (s, 3H), 3.12 (s, 3H), 2.62-2.58 (m, 2H), 2.41 (s, 3H), 2.31 (s, 3H), 2.09-2.05 (m, 1H), 1.55-1.47 (m, 2H), 1.01-0.92 (m, 2H), 0.75- 0.65 (m, 2H). |
| Example 162 | 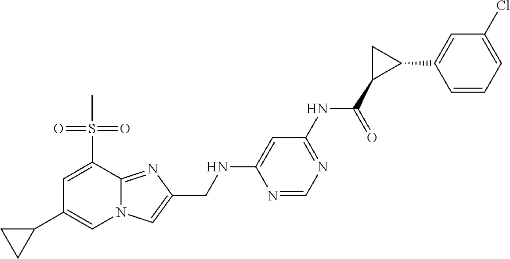 [SMILES: CS(=O)(=O)c1cc(C2CC2)cn2cc(CNc3cc(NC(=O)[C@H]4C[C@@H]4c4cccc(Cl)c4)ncn3)nc12] (1S,2S)-2-(3-chlorophenyl)-N-(6-(((6- cyclopropyl-8- (methylsulfonyl)imidazo[1,2-a]pyridin- 2-yl)methyl)amino)pyrimidin-4- yl)cyclopropane-1-carboxamide (I- 162) | 6-chloro-N-((6-cyclopropyl-8- (methylsulfonyl)imidazo[1,2-a]pyridin- 2-yl)methyl)pyrimidin-4-amine ESI-MS (M + H)+: 537.1, ¹H NMR (400 MHz, DMSO) δ 10.62 (s, 1H), 8.63 (d, J = 1.2 Hz, 1H), 8.20 (s, 1H), 7.99 (s, 1H), 7.81 (s, 1H), 7.56 (d, J = 1.6 Hz, 1H), 7.34-7.30 (m, 2H), 7.27-7.25 (m, 2H), 7.15 (d, J = 7.6 Hz, 1H), 4.65 (s, 2H), 3.54 (s, 3H), 2.43-2.36 (m, 2H), 2.09-2.05 (m, 1H), 1.49-1.45 (m, 1H), 1.43-1.38 (m, 1H), 1.00- 0.95 (m, 2H), 0.73-0.69 (m, 2H). ESI-MS [M + H]+: 537.1. |
| Example 163 | 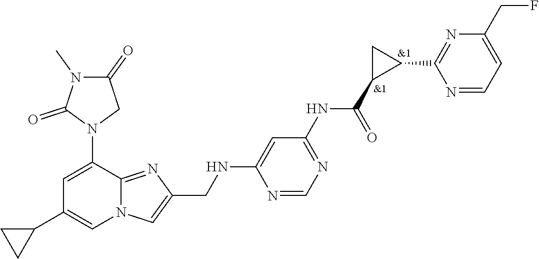 [SMILES: CN1C(=O)CN(c2cc(C3CC3)cn3cc(CNc4cc(NC(=O)[C@H]5C[C@@H]5c5nccc(CF)n5)ncn4)nc23)C1=O] rac-(1S*,2S*)-N-(6-(((6-cyclopropyl- 8-(3-methyl-2,4-dioxoimidazolidin-1- yl)imidazo[1,2-a]pyridin-2- yl)methyl)amino)pyrimidin-4-yl)-2-(4- (fluoromethyl)pyrimidin-2- yl)cyclopropane-1-carboxamide (I- 163) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 571.2, ¹H NMR (400 MHz, DMSO) δ 10.69 (s, 1H), 8.76 (d, J = 5.0 Hz, 1H), 8.21 (d, J = 22.2 Hz, 2H), 7.87 (s, 1H), 7.69 (s, 1H), 7.38 (d, J = 5.0 Hz, 1H), 7.32 (d, J = 11.9 Hz, 1H), 5.53 (s, 1H), 5.42 (s, 1H), 4.91 (s, 2H), 4.57 (s, 2H), 2.97 (s, 3H), 2.68- 2.62 (m, 2H), 1.95-1.92 (m, 1H), 1.57- 1.53 (m, 2H), 0.96-0.91 (m, 2H), 0.68- 0.61 (m, 2H). |
| Example 164 | 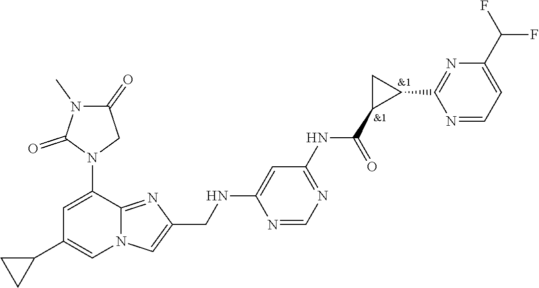 [SMILES: CN1C(=O)CN(c2cc(C3CC3)cn3cc(CNc4cc(NC(=O)[C@H]5C[C@@H]5c5nccc(C(F)F)n5)ncn4)nc23)C1=O] rac-(1S*,2S*)-N-(6-(((6-cyclopropyl- 8-(3-methyl-2,4-dioxoimidazolidin-1- yl)imidazo[1,2-a]pyridin-2- yl)methyl)amino)pyrimidin-4-yl)-2-(4- (difluoromethyl)pyrimidin-2- yl)cyclopropane-1-carboxamide (I- 164) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 589.2, ¹H NMR (400 MHz, DMSO) δ 10.74 (s, 1H), 8.95 (d, J = 5.0 Hz, 1H), 8.22 (d, J = 23.1 Hz, 2H), 7.90 (s, 1H), 7.69 (s, 1H), 7.62 (d, J = 5.0 Hz, 1H), 7.32 (d, J = 8.0 Hz, 2H), 7.08 (s, 0.29H), 6.95 (s, 0.48H), 6.81 (s, 0.27H), 4.91 (s, 2H), 4.58 (s, 2H), 2.97 (s, 3H), 2.76-2.60 (m, 2H), 1.98- 1.89(m, 1H), 1.66-1.48 (m, 2H), 1.05- 0.84 (m, 2H), 0.77-0.50 (m, 2H). |
| Example 165 | 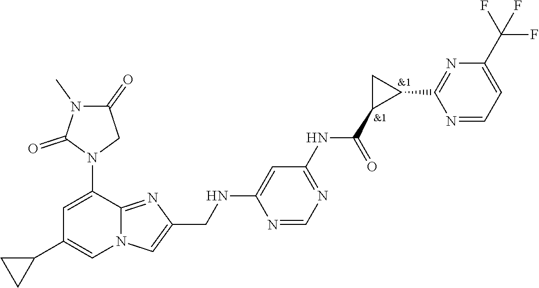 [SMILES: CN1C(=O)CN(c2cc(C3CC3)cn3cc(CNc4cc(NC(=O)[C@H]5C[C@@H]5c5nccc(C(F)(F)F)n5)ncn4)nc23)C1=O] rac-(1S*,2S*)-N-(6-(((6-cyclopropyl- 8-(3-methyl-2,4-dioxoimidazolidin-1- yl)imidazo[1,2-a]pyridin-2- yl)methyl)amino)pyrimidin-4-yl)-2-(4- (trifluoromethyl)pyrimidin-2- yl)cyclopropane-1-carboxamide (I- 165) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 607.2, ¹H NMR (400 MHz, DMSO) δ 10.74 (s, 1H), 9.08 (d, J = 5.0 Hz, 1H), 8.24-8.19 (m, 2H), 7.90 (s, 1H), 7.86 (d, J = 5.1 Hz, 1H), 7.69 (s, 1H), 7.33-7.31 (m, 2H), 4.91 (s, 2H), 4.58 (s, 2H), 2.97 (s, 3H), 2.77- 2.73 (m, 1H), 2.70-2.65 (m, 1H), 1.97-1.90 (m, 1H), 1.65-1.58 (m, 2H), 0.96-0.91 (m, 2H), 0.66-0.62 (m, 2H). |
| Example 166 | 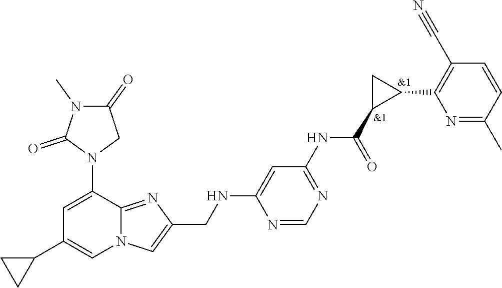 [SMILES: Cc1ccc(C#N)c([C@H]2C[C@@H]2C(=O)Nc2cc(NCc3cn4cc(C5CC5)cc(N5CC(=O)N(C)C5=O)c4n3)ncn2)n1] rac-(1S*,2S*)-2-(3-cyano-6- methylpyridin-2-yl)-N-(6-(((6- cyclopropyl-8-(3-methyl-2,4- dioxoimidazolidin-1-yl)imidazo[1,2- a]pyridin-2- yl)methyl)amino)pyrimidin-4- yl)cyclopropane-1-carboxamide (I- 166) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 577.2, ¹H NMR (400 MHz, DMSO) δ 10.71 (s, 1H), 8.24 (d, J = 1.0 Hz, 1H), 8.19 (s, 1H), 8.12 (d, J = 8.0 Hz, 1H), 7.90 (s, 1H), 7.69 (s, 1H), 7.33-7.29 (m, 3H), 4.91 (s, 2H), 4.58 (s, 2H), 2.97 (s, 3H), 2.81-2.76 (m, 1H), 2.74-2.67 (m, 1H), 2.51 (s, 3H), 1.97-1.90 (m, 1H), 1.63-1.55 (m, 2H), 0.96-0.91 (m, 2H), 0.66-0.62 (m, 2H). |
| Example 167 | 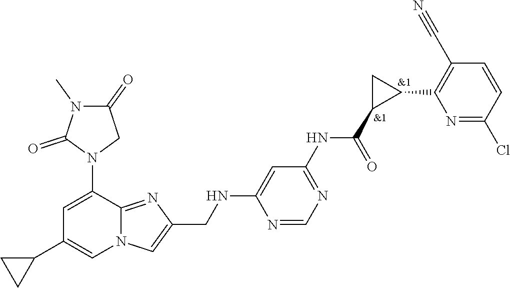 [SMILES: CN1C(=O)CN(c2cc(C3CC3)cn3cc(CNc4cc(NC(=O)[C@H]5C[C@@H]5c5nc(Cl)ccc5C#N)ncn4)nc23)C1=O] rac-(1S*,2S*)-2-(6-chloro-3- cyanopyridin-2-yl)-N-(6-(((6- cyclopropyl-8-(3-methyl-2,4- dioxoimidazolidin-1-yl)imidazo[1,2- a]pyridin-2- yl)methyl)amino)pyrimidin-4- yl)cyclopropane-1-carboxamide (I- 167) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 597.2, ¹H NMR (400 MHz, DMSO) δ 10.73 (s, 1H), 8.32 (d, J = 8.3 Hz, 1H), 8.24-8.19 (m, 2H), 7.90 (s, 1H), 7.69 (s, 1H), 7.59 (d, J = 8.3 Hz, 1H), 7.33 (d, J = 3.8 Hz, 2H), 4.90 (s, 2H), 4.58 (s, 2H), 2.97 (s, 3H), 2.77-2.74 (m, 2H), 1.93-1.90 (m, 1H), 1.66-1.58 (m, 2H), 0.94-0.91 (m, 2H), 0.66-0.62 (m, 2H). ESI-MS [M + H]+: 597.1 |
| Example 168 | 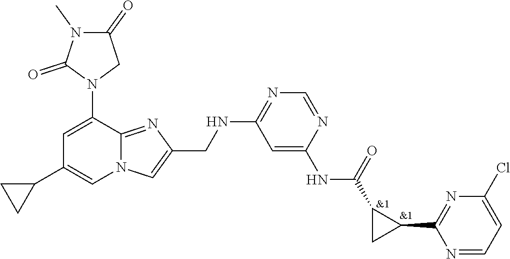 [SMILES: CN1C(=O)CN(c2cc(C3CC3)cn3cc(CNc4cc(NC(=O)[C@H]5C[C@@H]5c5nccc(Cl)n5)ncn4)nc23)C1=O] rac-(1S*,2S*)-2-(4-chloropyrimidin-2- yl)-N-(6-(((6-cyclopropyl-8-(3-methyl- 2,4-dioxoimidazolidin-1- yl)imidazo[1,2-a]pyridin-2- yl)methyl)amino)pyrimidin-4- yl)cyclopropane-1-carboxamide (I- 168) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 573.2, ¹H NMR (400 MHz, DMSO) δ 10.71 (s, 1H), 8.68 (d, J = 5.4 Hz, 1H), 8.35-8.09 (m, 2H), 7.89 (s, 1H), 7.69 (s, 1H), 7.55 (d, J = 5.3 Hz, 1H), 7.42-7.14 (m, 2H), 4.91 (s, 2H), 4.58 (s, 2H), 2.96 (d, J = 10.1 Hz, 3H), 2.77-2.62 (m, 1H), 2.62- 2.53 (m, 1H), 2.03-1.78 (m, 1H), 1.67- 1.38 (m, 2H), 1.06-0.77 (m, 2H), 0.76-0.51 (m, 2H). |
| Example 169 | 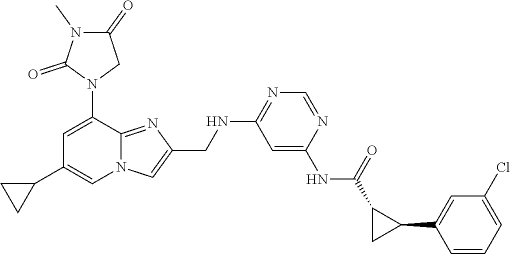 [SMILES: CN1C(=O)CN(c2cc(C3CC3)cn3cc(CNc4cc(NC(=O)[C@H]5C[C@@H]5c5cccc(Cl)c5)ncn4)nc23)C1=O] (1S,2S)-2-(3-chlorophenyl)-N-(6-(((6- cyclopropyl-8-(3-methyl-2,4- dioxoimidazolidin-1-yl)imidazo[1,2- a]pyridin-2- yl)methyl)amino)pyrimidin-4- yl)cyclopropane-1-carboxamide (I- 169) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 571.1, ¹H NMR (400 MHz, DMSO) δ 10.67 (s, 1H), 8.31 (d, J = 1.0 Hz, 1H), 8.25 (s, 1H), 7.96 (s, 1H), 7.75 (s, 1H), 7.39-7.31 (m, 5H), 7.21 (d, J = 7.6 Hz, 1H), 4.97 (s, 2H), 4.64 (s, 2H), 3.04 (s, 3H), 2.48-2.39 (m, 2H), 2.03-1.97 (m, 1H), 1.54- 1.50 (m, 1H), 1.50-1.45 (d, J = 7.6 Hz, 1H), 1.03-0.98 (m, 2H), 0.72-0.68 (m, 1H). |
| Example 170 | 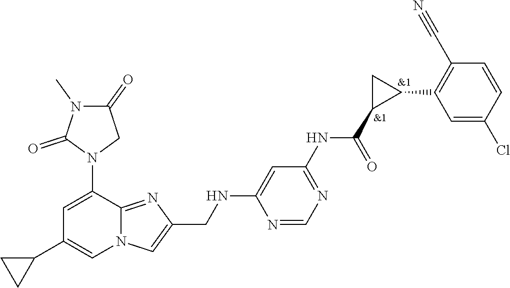 [SMILES: CN1C(=O)CN(c2cc(C3CC3)cn3cc(CNc4cc(NC(=O)[C@H]5C[C@@H]5c5cc(Cl)ccc5C#N)ncn4)nc23)C1=O] rac-(1S*,2S*)-2-(5-chloro-2- cyanophenyl)-N-(6-(((6-cyclopropyl-8- (3-methyl-2,4-dioxoimidazolidin-1- yl)imidazo[1,2-a]pyridin-2- yl)methyl)amino)pyrimidin-4- yl)cyclopropane-1-carboxamide (I- 170) | 1-(2-(((6-bromopyrimidin-4- yl)amino)methyl)-6- cyclopropylimidazo[1,2-a]pyridin-8- yl)-3-methylimidazolidine-2,4-dione ESI-MS (M + H)+: 596.1, ¹H NMR (400 MHz, DMSO) δ 10.67 (s, 1H), 8.24 (d, J = 1.1 Hz, 1H), 8.20 (s, 1H), 7.91 (s, 1H), 7.86 (d, J = 8.3 Hz, 1H), 7.69 (s, 1H), 7.54-7.51 (m, 1H), 7.41 (d, J = 1.9 Hz, 1H), 7.33 (d, J = 2.8 Hz, 2H), 4.91 (s, 2H), 4.58 (s, 2H), 2.97 (s, 3H), 2.62- 2.57 (m, 1H), 2.49-2.45 (m, 1H), 1.97-1.90 (m, 1H), 1.62-1.53 (m, 2H), 0.96-0.91 (m, 2H), 0.66-0.62 (m, 2H). |
| Example 171 | 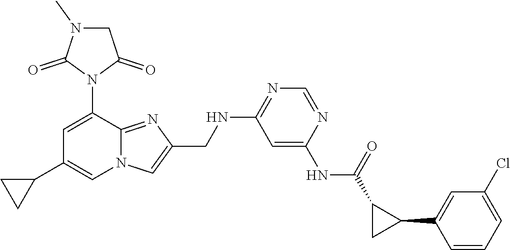 [SMILES: CN1CC(=O)N(c2cc(C3CC3)cn3cc(CNc4cc(NC(=O)[C@H]5C[C@@H]5c5cccc(Cl)c5)ncn4)nc23)C1=O] (1S,2S)-2-(3-chlorophenyl)-N-(6-(((6- cyclopropyl-8-(3-methyl-2,5- dioxoimidazolidin-1-yl)imidazo[1,2- a]pyridin-2- yl)methyl)amino)pyrimidin-4- yl)cyclopropane-1-carboxamide (I- 171) | 3-(2-(((6-bromopyrimidin-4- yl)amino)methyl)-6- cyclopropylimidazo[1,2-a]pyridin-8- yl)-1-methylimidazolidine-2,4-dione ESI-MS (M + H)+: 571.3, ¹H NMR (400 MHz, DMSO) δ 10.57 (s, 1H), 8.16 (s, 1H), 7.79 (s, 1H), 7.48 (d, J = 21.6 Hz, 2H), 7.33-7.17 (m, 4H), 7.12 (d, J = 7.6 Hz, 1H), 5.92 (s, 1H), 5.47 (d, J = 8.5 Hz, 1H), 4.51 (s, 2H), 3.09-3.07 (m, 1H), 2.78-2.59 (m, 1H), 2.53(s, 3H), 2.40-2.27 (m, 1H), 1.47-1.42 (m, 1H), 1.41-1.34 (m, 1H), 0.83-0.81 (m, 2H), 0.66-0.52 (m, 2H). |
| Example 172 | 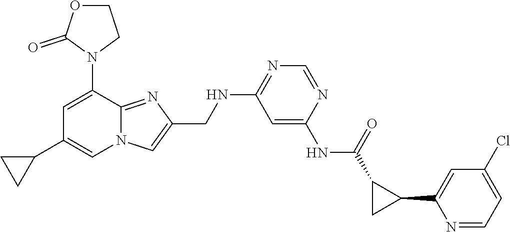 [SMILES: O=C(Nc1cc(NCc2cn3cc(C4CC4)cc(N4CCOC4=O)c3n2)ncn1)[C@H]1C[C@@H]1c1cc(Cl)ccn1] (1S,2S)-2-(4-chloropyridin-2-yl)-N-(6- (((6-cyclopropyl-8-(2-oxooxazolidin-3- yl)imidazo[1,2-a]pyridin-2- yl)methyl)amino)pyrimidin-4- yl)cyclopropane-1-carboxamide (I- 172) | 3-(2-(((6-bromopyrimidin-4- yl)amino)methyl)-6- cyclopropylimidazo[1,2-a]pyridin-8- yl)oxazolidin-2-one ESI-MS (M + H)+: 545.2, ¹H NMR (400 MHz, MeOD) δ 8.35 (d, J = 5.4 Hz, 1H), 8.16 (dd, J = 6.5, 0.8 Hz, 2H), 7.70 (s, 1H), 7.44 (d, J = 1.8 Hz, 1H), 7.34 (d, J = 0.9 Hz, 1H), 7.26 (dd, J = 5.4, 2.0 Hz, 1H), 7.21 (d, J = 1.5 Hz, 1H), 4.69-4.59 (m, 4H), 4.32-4.28 (m, 2H), 2.63-2.58 (m, 1H), 2.47-2.43 (m, 1H), 1.97- 1.93 (m, 1H), 1.64-1.59 (m, 2H), 1.00- 0.94 (m, 2H), 0.75-0.71 (m, 2H). |
| Example 173 | 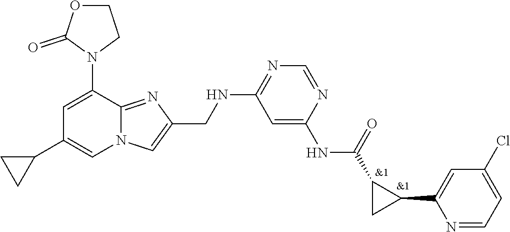 [SMILES: O=C(Nc1cc(NCc2cn3cc(C4CC4)cc(N4CCOC4=O)c3n2)ncn1)[C@H]1C[C@@H]1c1cc(Cl)ccn1] rac-(1S*,2S*)-2-(4-chloropyridin-2- yl)-N-(6-(((6-cyclopropyl-8-(2- oxooxazolidin-3-yl)imidazo[1,2- a]pyridin-2- yl)methyl)amino)pyrimidin-4- yl)cyclopropane-1-carboxamide (I- 173) | 3-(2-(((6-bromopyrimidin-4- yl)amino)methyl)-6- cyclopropylimidazo[1,2-a]pyridin-8- yl)oxazolidin-2-one ESI-MS (M + H)+: 545.2, ¹H NMR (400 MHz, MeOD) δ 8.35 (d, J = 5.4 Hz, 1H), 8.16 (dd, J = 6.5, 0.8 Hz, 2H), 7.70 (s, 1H), 7.44 (d, J = 1.8 Hz, 1H), 7.34 (d, J = 0.9 Hz, 1H), 7.26 (dd, J = 5.4, 2.0 Hz, 1H), 7.21 (d, J = 1.5 Hz, 1H), 4.69-4.59 (m, 4H), 4.32-4.28 (m, 2H), 2.63-2.58 (m, 1H), 2.47-2.43 (m, 1H), 1.97- 1.93 (m, 1H), 1.64-1.59 (m, 2H), 1.00- 0.94 (m, 2H), 0.75-0.71 (m, 2H). |
| Example 174 | 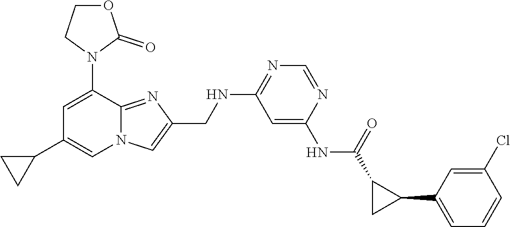 [SMILES: O=C(Nc1cc(NCc2cn3cc(C4CC4)cc(N4CCOC4=O)c3n2)ncn1)[C@H]1C[C@@H]1c1cccc(Cl)c1] (1S,2S)-2-(3-chlorophenyl)-N-(6-(((6- cyclopropyl-8-(2-oxooxazolidin-3- yl)imidazo[1,2-a]pyridin-2- yl)methyl)amino)pyrimidin-4- yl)cyclopropane-1-carboxamide (I- 174) | 3-(2-(((6-chloropyrimidin-4- yl)amino)methyl)-6- cyclopropylimidazo[1,2-a]pyridin-8- yl)oxazolidin-2-one ESI-MS (M + H)+: 544.2, ¹H NMR (400 MHz, DMSO) δ 10.61 (s, 1H), 8.24 (d, J = 1.2 Hz, 1H), 8.19 (s, 1H), 7.89 (s, 1H), 7.67 (s, 1H), 7.41-6.94 (m, 6H), 4.57-4.44 (m, 6H), 2.46-2.32 (m, 2H), 1.93-1.89 (m, 1H), 1.52-1.44 (m, 1H), 1.44-1.35 (m, 1H), 0.98-0.86 (m, 2H), 0.71-0.52 (m, 2H). |
| Example 175 | 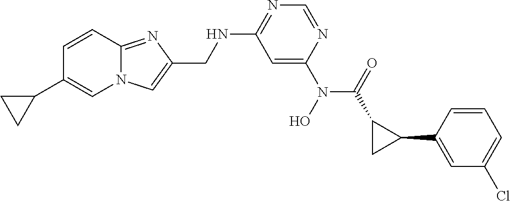 [SMILES: O=C([C@H]1C[C@@H]1c1cccc(Cl)c1)N(O)c1cc(NCc2cn3cc(C4CC4)ccc3n2)ncn1] (1S,2S)-2-(3-chlorophenyl)-N-(6-(((6- cyclopropylimidazo[1,2-a]pyridin-2- yl)methyl)amino)pyrimidin-4-yl)-N- hydroxycyclopropane-1-carboxamide (I-175) | 6-bromo-N-((6- cyclopropylimidazo[1,2-a]pyridin-2- yl)methyl)pyrimidin-4-amine ESI-MS (M + H)+: 475.1, ¹H NMR (400 MHz, DMSO) δ 10.39 (s, 1H), 8.28 (s, 1H), 8.04-7.96 (m, 1H), 7.61 (s, 2H), 7.44-7.06 (m, 5H), 6.97-6.92(m, 1H), 5.71 (s, 1H), 4.60-4.49 (m, 2H), 2.60- 2.55 (m, 1H), 2.48-2.38 (m, 1H), 1.91- 1.87 (m, 1H), 1.61-1.42 (m, 2H), 0.92- 0.89 (m, 2H), 0.69-0.61 (m, 2H). |
| Example 176 | 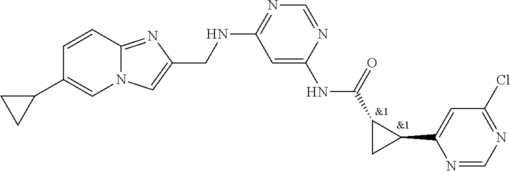 [SMILES: O=C(Nc1cc(NCc2cn3cc(C4CC4)ccc3n2)ncn1)[C@H]1C[C@@H]1c1cc(Cl)ncn1] rac-(1S*,2S*)-2-(6-chloropyrimidin-4- yl)-N-(6-(((6-cyclopropylimidazo[1,2- a]pyridin-2- yl)methyl)amino)pyrimidin-4- yl)cyclopropane-1-carboxamide (I- 176) | 6-bromo-N-((6- cyclopropylimidazo[1,2-a]pyridin-2- yl)methyl)pyrimidin-4-amine ESI-MS (M + H)+: 461.1, ¹H NMR (400 MHz, DMSO) δ 10.70 (s, 1H), 8.89 (s, 1H), 8.29 (s, 1H), 8.19 (s, 1H), 7.87 (s, 2H), 7.60 (s, 1H), 7.36 (d, J = 9.3 Hz, 1H), 7.26 (s, 1H), 6.97-6.94 (m, 1H), 4.55 (s, 2H), 2.76-2.68 (m, 1H), 2.67- 2.59 (m, 1H), 1.96-1.85 (m, 1H), 1.64- 1.52 (m, 2H), 0.95-0.86 (m, 2H), 0.69-0.61 (m, 2H). |
| Example 177 | 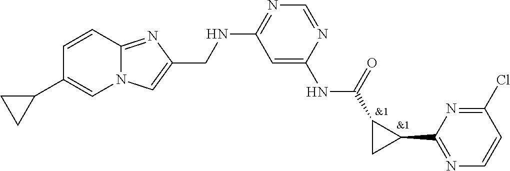 [SMILES: O=C(Nc1cc(NCc2cn3cc(C4CC4)ccc3n2)ncn1)[C@H]1C[C@@H]1c1nccc(Cl)n1] rac-(1S*,2S*)-2-(4-chloropyrimidin-2- yl)-N-(6-(((6-cyclopropylimidazo[1,2- a]pyridin-2- yl)methyl)amino)pyrimidin-4- yl)cyclopropane-1-carboxamide (I- 177) | 6-bromo-N-((6- cyclopropylimidazo[1,2-a]pyridin-2- yl)methyl)pyrimidin-4-amine ESI-MS (M + H)+: 461.1, ¹H NMR (400 MHz, DMSO) δ 10.70 (s, 1H), 8.68 (d, J = 5.3 Hz, 1H), 8.30 (s, 1H), 8.19 (s, 1H), 7.87 (s, 1H), 7.61 (s, 1H), 7.55 (d, J = 5.3 Hz, 1H), 7.37 (d, J = 9.3 Hz, 1H), 7.27 (s, 1H), 6.95 (dd, J = 9.3, 1.6 Hz, 1H), 4.57 (s, 2H), 2.80-2.63 (m, 1H), 2.64-2.50 (m, 11H), 1.90-1.87 (m, 1H), 1.59-1.54 (m, 2H), 0.99-0.79 (m, 2H), 0.77-0.51 (m, 2H). |
| Example 178 | 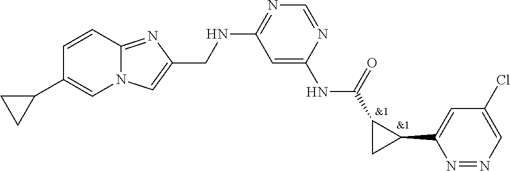 [SMILES: O=C(Nc1cc(NCc2cn3cc(C4CC4)ccc3n2)ncn1)[C@H]1C[C@@H]1c1cc(Cl)cnn1] rac-(1S*,2S*)-2-(5-chloropyridazin-3- yl)-N-(6-(((6-cyclopropylimidazo[1,2- a]pyridin-2- yl)methyl)amino)pyrimidin-4- yl)cyclopropane-1-carboxamide (I- 178) | 6-bromo-N-((6- cyclopropylimidazo[1,2-a]pyridin-2- yl)methyl)pyrimidin-4-amine ESI-MS (M + H)+: 461.1, ¹H NMR (400 MHz, DMSO) δ 10.69 (s, 1H), 9.24 (d, J = 2.3 Hz, 1H), 8.30 (s, 1H), 8.20 (s, 1H), 8.04 (d, J = 2.2 Hz, 1H), 7.88 (s, 1H), 7.61 (s, 1H), 7.37 (d, J = 9.3 Hz, 1H), 7.28 (s, 1H), 6.95 (d, J = 9.3 Hz, 1H), 4.57 (s, 2H), 2.78-2.64 (m, 2H), 1.96-1.85 (m, 1H), 1.75-1.65 (m, 1H), 1.64-1.56 (m, 1H), 0.97-0.84 (m, 2H), 0.71-0.58 (m, 2H). |
| Example 179 | 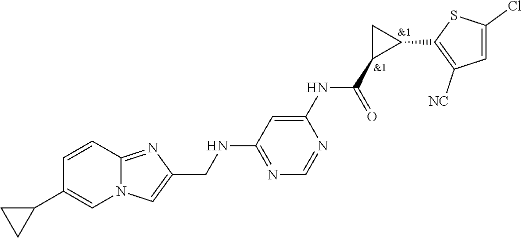 [SMILES: N#Cc1cc(Cl)sc1[C@H]1C[C@@H]1C(=O)Nc1cc(NCc2cn3cc(C4CC4)ccc3n2)ncn1] rac-(1S*,2S*)-2-(5-chloro-3- cyanothiophen-2-yl)-N-(6-(((6- cyclopropylimidazo[1,2-a]pyridin-2- yl)methyl)amino)pyrimidin-4- yl)cyclopropane-1-carboxamide (I- 179) | 6-bromo-N-((6- cyclopropylimidazo[1,2-a]pyridin-2- yl)methyl)pyrimidin-4-amine ESI-MS (M + H)+: 490.1, ¹H NMR (400 MHz, DMSO) δ 10.79 (s, 1H), 8.30 (s, 1H), 8.22 (s, 1H), 7.91 (s, 1H), 7.61 (s, 1H), 7.52 (s, 1H), 7.37 (d, J = 9.3 Hz, 1H), 7.27 (s, 1H), 7.01-6.93 (m, 1H), 4.57 (s, 2H), 2.73-2.65 (m, 2H), 1.94- 1.87 (m, 1H), 1.65-1.62 (m, 1H), 1.49- 1.44 (m, 1H), 0.96-0.86 (m, 2H), 0.72- 0.59 (m, 2H). |
| Example 180 | 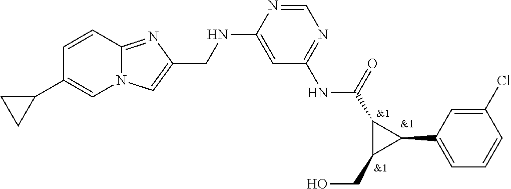 [SMILES: O=C(Nc1cc(NCc2cn3cc(C4CC4)ccc3n2)ncn1)[C@H]1[C@H](CO)[C@@H]1c1cccc(Cl)c1] rac-(1R*,2S*,3R*)-2-(3- chlorophenyl)-N-(6-(((6- cyclopropylimidazo[1,2-a]pyridin-2- yl)methyl)amino)pyrimidin-4-yl)-3- (hydroxymethyl)cyclopropane-1- carboxamide (I-180) | 6-bromo-N-((6- cyclopropylimidazo[1,2-a]pyridin-2- yl)methyl)pyrimidin-4-amine ESI-MS (M + H)+: 489.2, ¹H NMR (400 MHz, DMSO) δ 10.64 (s, 1H), 8.29 (s, 1H), 8.22 (s, 1H), 7.86 (s, 1H), 7.61 (s, 1H), 7.46-7.22 (m, 6H), 7.01-6.92 (m, 1H), 4.65-4.60 (m, 1H), 4.57 (s, 2H), 3.28-3.14 (m, 1H), 3.14-3.02 (m, 1H), 2.68-2.60 (m, 1H), 2.59-2.50 (m, 1H), 2.00-1.78 (m, 2H), 1.02-0.80 (m, 2H), 0.70-0.60 (m, 2H). |
| Example 181 | 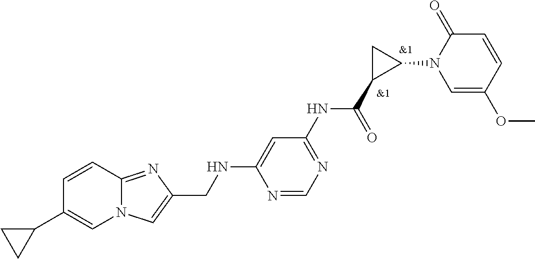 [SMILES: COc1ccc(=O)n([C@H]2C[C@@H]2C(=O)Nc2cc(NCc3cn4cc(C5CC5)ccc4n3)ncn2)c1] rac-(1S*,2S*)-N-(6-(((6- cyclopropylimidazo[1,2-a]pyridin-2- yl)methyl)amino)pyrimidin-4-yl)-2-(5- methoxy-2-oxopyridin-1 (2H)- yl)cyclopropane-1-carboxamide (I- 181) | 6-bromo-N-((6- cyclopropylimidazo[1,2-a]pyridin-2- yl)methyl)pyrimidin-4-amine ESI-MS (M + H)+: 472.2, ¹H NMR (400 MHz, DMSO) δ 10.70 (s, 1H), 8.30 (s, 1H), 8.22 (s, 1H), 7.90 (s, 1H), 7.61 (s, 1H), 7.38-7.29 (m, 3H), 7.07 (d, J = 2.6 Hz, 1H), 6.95 (d, J = 9.1 Hz, 1H), 6.37 (d, J = 9.9 Hz, 1H), 4.58 (s, 2H), 3.67 (s, 3H), 3.54-3.49 (m, 1H), 2.35-2.29 (m, 1H), 1.94-1.87 (m, 1H), 1.76- 1.71 (m, 1H), 1.50-1.45 (m, 1H), 0.93- 0.88 (m, 2H), 0.67-0.64 (m, 2H). |
| Example 182 | 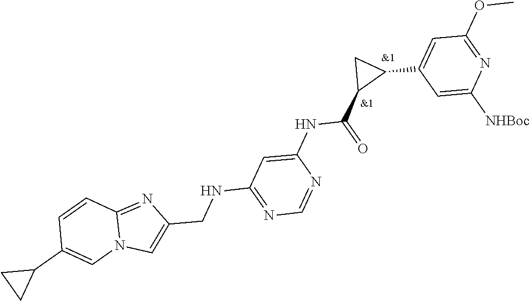 [SMILES: COc1cc([C@H]2C[C@@H]2C(=O)Nc2cc(NCc3cn4cc(C5CC5)ccc4n3)ncn2)cc(NC(=O)OC(C)(C)C)n1] rac-tert-butyl (4-((1S*,2S*)-2-((6-(((6- cyclopropylimidazo[1,2-a]pyridin-2- yl)methyl)amino)pyrimidin-4- yl)carbamoyl)cyclopropyl)-6- methoxypyridin-2-yl)carbamate (I-182) | 6-bromo-N-((6- cyclopropylimidazo[1,2-a]pyridin-2- yl)methyl)pyrimidin-4-amine ESI-MS (M + H)+: 571.3, ¹H NMR (400 MHz, DMSO) δ 10.66 (s, 1H), 9.50 (s, 1H), 8.29 (s, 1H), 8.19 (s, 1H), 7.86 (s, 1H), 7.60 (s, 1H), 7.37 (d, J = 9.3 Hz, 1H), 7.27 (s, 1H), 7.12 (s, 1H), 6.95 (dd, J = 9.3, 1.7 Hz, 1H), 6.29 (d, J = 0.7 Hz, 1H), 4.56 (s, 2H), 3.77 (s, 3H), 2.48- 2.42 (m, 1H), 2.35-2.29 (m, 1H), 1.94- 1.86 (m, 1H), 1.46 (s, 9H), 1.46-1.41 (m, 1H), 1.37-1.30 (m, 1H), 0.94- 0.87 (m, 2H), 0.68-0.63 (m, 2H). |
| Example 183 | 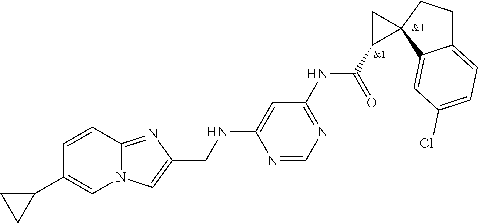 [SMILES: O=C(Nc1cc(NCc2cn3cc(C4CC4)ccc3n2)ncn1)[C@@H]1C[C@]12CCc1ccc(Cl)cc12] rac-(1R*,2R*)-6′-chloro-N-(6-(((6- cyclopropylimidazo[1,2-a]pyridin-2- yl)methyl)amino)pyrimidin-4-yl)-2′,3′- dihydrospiro[cyclopropane-1,1′- indene]-2-carboxamide (I-183) | 6-bromo-N-((6- cyclopropylimidazo[1,2-a]pyridin-2- yl)methyl)pyrimidin-4-amine ESI-MS (M + H)+: 485.3, ¹H NMR (400 MHz, DMSO) δ 10.49 (s, 1H), 8.30 (s, 1H), 8.17 (s, 1H), 7.84 (s, 1H), 7.61 (s, 1H), 7.37 (d, J = 9.3 Hz, 1H), 7.31 (s, 1H), 7.26-7.14 (m, 2H), 6.96 (dd, J = 9.3, 1.6 Hz, 1H), 6.90 (d, J = 1.6 Hz, 1H), 4.57 (s, 2H), 3.05-2.72 (m, 2H), 2.48-2.41 (m, 1H), 2.15-2.10 (m, 2H), 1.90-1.85 (m, 1H), 1.56-1.51 (m, 2H), 1.04-0.83 (m, 2H), 0.67- 0.64 (m, 2H). |
| Example 184 | 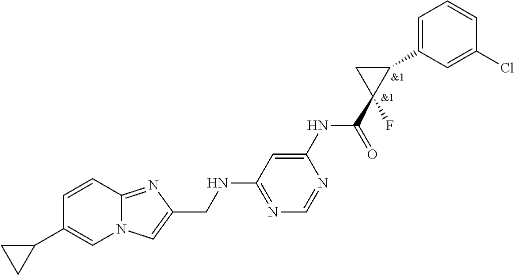 [SMILES: O=C(Nc1cc(NCc2cn3cc(C4CC4)ccc3n2)ncn1)[C@@]1(F)C[C@@H]1c1cccc(Cl)c1] rac-(1S*,2S*)-2-(3-chlorophenyl)-N- (6-(((6-cyclopropylimidazo[1,2- a]pyridin-2- yl)methyl)amino)pyrimidin-4-yl)-1- fluorocyclopropane-1-carboxamide (I- 184) | 6-bromo-N-((6- cyclopropylimidazo[1,2-a]pyridin-2- yl)methyl)pyrimidin-4-amine ESI-MS (M + H)+: 477.1, ¹H NMR (400 MHz, DMSO) δ 10.12 (s, 1H), 8.28 (d, J = 21.0 Hz, 2H), 8.01 (s, 1H), 7.63 (s, 1H), 7.45 (s, 1H), 7.38-7.24 (m, 5H), 6.96 (d, J = 9.1 Hz, 1H), 4.60 (s, 2H), 2.95-2.86 (m, 1H), 2.23-2.04 (m, 1H), 1.99-1.81 (m, 2H), 1.00-0.82 (m, 2H), 0.70-0.61 (m, 2H). |
| Example 185 | 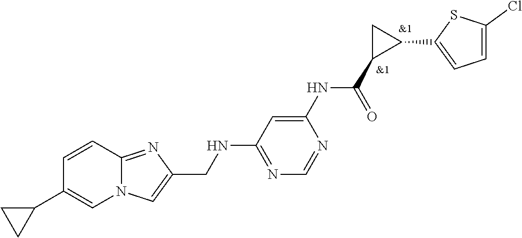 [SMILES: O=C(Nc1cc(NCc2cn3cc(C4CC4)ccc3n2)ncn1)[C@H]1C[C@@H]1c1ccc(Cl)s1] rac-(1S*,2S*)-2-(5-chlorothiophen-2- yl)-N-(6-(((6-cyclopropylimidazo[1,2- a]pyridin-2- yl)methyl)amino)pyrimidin-4- yl)cyclopropane-1-carboxamide (I- 185) | 6-bromo-N-((6- cyclopropylimidazo[1,2-a]pyridin-2- yl)methyl)pyrimidin-4-amine ESI-MS (M + H)+: 465.2, ¹H NMR (400 MHz, DMSO) δ = 10.12 (s, 1H), 8.31 (s, 1H), 8.25 (s, 1H), 8.01 (s, 1H), 7.63 (s, 1H), 7.45 (s, 1H), 7.39-7.30(m, 3H), 6.97 (d, J = 9.1, 1H), 4.60 (s, 2H), 3.31 (s, 1H), 2.99-2.84(m, 1H), 2.23-2.05 (m, 1H), 2.03-1.76 (m, 2H), 0.99-0.80 (m, 2H), 0.72-0.58 (m, 2H). |
| Example 186 | 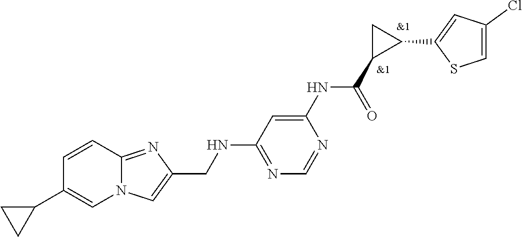 [SMILES: O=C(Nc1cc(NCc2cn3cc(C4CC4)ccc3n2)ncn1)[C@H]1C[C@@H]1c1cc(Cl)cs1] rac-(1S*,2S*)-2-(4-chlorothiophen-2- yl)-N-(6-(((6-cyclopropylimidazo[1,2- a]pyridin-2- yl)methyl)amino)pyrimidin-4- yl)cyclopropane-1-carboxamide (I- 186) | 6-bromo-N-((6- cyclopropylimidazo[1,2-a]pyridin-2- yl)methyl)pyrimidin-4-amine ESI-MS (M + H)+: 465.1, ¹H NMR (400 MHz, DMSO) δ = 10.68 (s, 1H), 8.30 (s, 1H), 8.20 (s, 1H), 7.88 (s, 1H), 7.60 (s, 1H), 7.41-7.32 (m, 2H), 7.26 (s, 1H), 7.02-6.88 (m, 2H), 4.57 (s, 2H), 3.31- 3.30 (m, 1H), 2.41-2.30 (m, 1H), 1.94- 1.86 (m, 1H), 1.52-1.45 (m, 1H), 1.38- 1.30 (m, 1H), 0.96-0.85 (m, 2H), 0.69- 0.61 (m, 2H). |
| Example 187 | 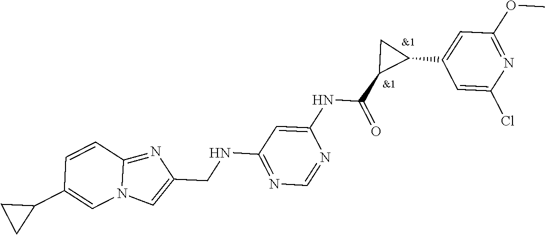 [SMILES: COc1cc([C@H]2C[C@@H]2C(=O)Nc2cc(NCc3cn4cc(C5CC5)ccc4n3)ncn2)cc(Cl)n1] rac-(1S*,2S*)-2-(2-chloro-6- methoxypyridin-4-yl)-N-(6-(((6- cyclopropylimidazo[1,2-a]pyridin-2- yl)methyl)amino)pyrimidin-4- yl)cyclopropane-1-carboxamide (I- 187) | 6-bromo-N-((6- cyclopropylimidazo[1,2-a]pyridin-2- yl)methyl)pyrimidin-4-amine ESI-MS (M + H)+: 490.2, ¹H NMR (400 MHz, DMSO) δ 10.61 (s, 1H), 8.30 (s, 1H), 8.20 (s, 1H), 7.88 (s, 1H), 7.61 (s, 1H), 7.37 (d, J = 9.2 Hz, 1H), 7.25 (s, 1H), 6.99-6.91 (m, 2H), 6.68 (s, 1H), 4.57 (s, 2H), 3.82 (s, 3H), 2.48-2.44 (m, 1H), 2.42-2.37 (m, 1H), 1.96- 1.86 (m, 1H), 1.55-1.47 (m, 2H), 0.95- 0.86 (m, 2H), 0.69-0.63 (m, 2H). |
| Example 188 | 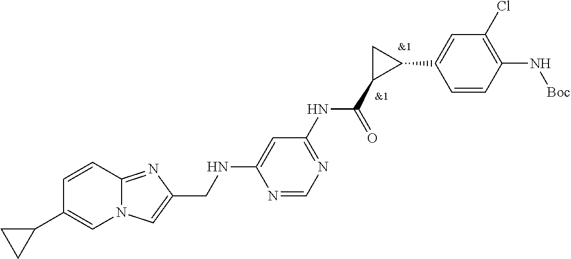 [SMILES: CC(C)(C)OC(=O)Nc1ccc([C@H]2C[C@@H]2C(=O)Nc2cc(NCc3cn4cc(C5CC5)ccc4n3)ncn2)cc1Cl] rac-tert-butyl (2-chloro-4-((1S*,2S*)- 2-((6-(((6-cyclopropylimidazo[1,2- a]pyridin-2- yl)methyl)amino)pyrimidin-4- yl)carbamoyl)cyclopropyl)phenyl) carbamate (I-188) | 6-chloro-N-((6- cyclopropylimidazo [1,2-a]pyridin-2- yl)methyl)pyrimidin-4-amine ESI-MS (M + H)+: 574.2, ¹H NMR (400 MHz, DMSO-d₆) δ 10.61 (s, 1H), 8.61 (s, 1H), 8.30 (s, 1H), 8.18 (s, 1H), 7.87 (s, 1H), 7.60 (s, 1H), 7.43 (d, J = 8.4 Hz, 1H), 7.37 (d, J = 9.2 Hz, 1H), 7.27 (d, J = 1.9 Hz, 2H), 7.11-7.06 (m, 1H), 6.99- 6.93 (m, 1H), 4.56 (s, 2H), 2.39-2.29 (m, 2H), 1.94-1.86 (m, 1H), 1.44 (s, 9H), 1.43-1.40 (m, 1H), 1.38-1.33 (m, 1H), 0.93-0.87 (m, 2H), 0.68- 0.62 (m, 2H). |
| Example 189 | 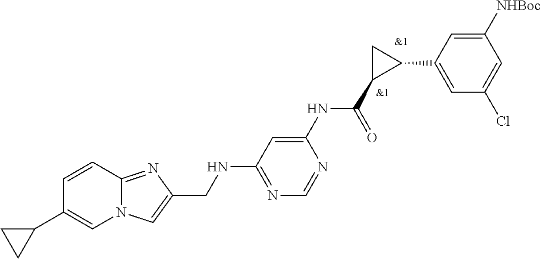 [SMILES: CC(C)(C)OC(=O)Nc1cc(Cl)cc([C@H]2C[C@@H]2C(=O)Nc2cc(NCc3cn4cc(C5CC5)ccc4n3)ncn2)c1] rac-tert-butyl (3-chloro-5-((1S*,2S*)- 2-((6-(((6-cyclopropylimidazo[1,2- a]pyridin-2- yl)methyl)amino)pyrimidin-4- yl)carbamoyl)cyclopropyl)phenyl) carbamate (I-189) | 6-chloro-N-((6- cyclopropylimidazo[1,2-a]pyridin-2- yl)methyl)pyrimidin-4-amine ESI-MS (M + H)+: 574.3, ¹H NMR (400 MHz, DMSO) δ 10.62 (s, 1H), 9.53 (s, 1H), 8.29 (s, 1H), 8.19 (s, 1H), 7.85 (s, 1H), 7.60 (s, 1H), 7.39-7.36 (m, 2H), 7.26 (s, 1H), 7.21 (s, 1H), 6.97-6.94 (m, 1H), 6.84 (s, 1H), 4.56 (s, 2H), 3.32 (s, 11H), 2.51- 2.50 (m, 13H), 2.35-2.30 (m, 2H), 1.93-1.88 (m, 1H), 1.47 (s, 9H), 1.45-1.40 (m, 1H), 1.33-1.28 (m, 1H), 0.93-0.88 (m, 2H), 0.68-0.64 (m, 2H). |
| Example 190 | 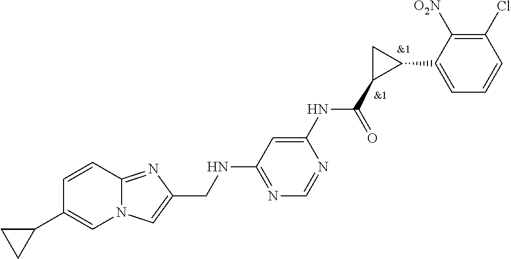 [SMILES: O=C(Nc1cc(NCc2cn3cc(C4CC4)ccc3n2)ncn1)[C@H]1C[C@@H]1c1cccc(Cl)c1[N+](=O)[O-]] rac-(1S*,2S*)-2-(3-chloro-2- nitrophenyl)-N-(6-(((6- cyclopropylimidazo[1,2-a]pyridin-2- yl)methyl)amino)pyrimidin-4- yl)cyclopropane-1-carboxamide (I- 190) | 6-chloro-N-((6- cyclopropylimidazo[1,2-a]pyridin-2- yl)methyl)pyrimidin-4-amine ESI-MS (M + H)+: 504.1, ¹H NMR (400 MHz, DMSO) δ 10.68 (s, 1H), 8.31 (s, 1H), 8.21 (s, 1H), 7.89 (s, 1H), 7.71- 7.57 (m, 3H), 7.43-7.33 (m, 2H), 7.26 (s, 1H), 7.00 (d, J = 9.3 Hz, 1H), 4.58 (s, 2H), 2.43-2.35 (m, 1H), 2.25-2.17 (m, 1H), 1.96-1.86 (m, 1H), 1.55- 1.40 (m, 2H), 0.95-0.88 (m, 2H), 0.70- 0.63 (m, 2H) |
| Example 191 | 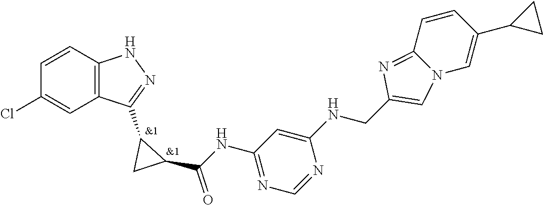 [SMILES: O=C(Nc1cc(NCc2cn3cc(C4CC4)ccc3n2)ncn1)[C@H]1C[C@@H]1c1n[nH]c2ccc(Cl)cc12] rac-(1S*,2S*)-2-(5-chloro-1H-indazol- 3-yl)-N-(6-(((6- cyclopropylimidazo[1,2-a]pyridin-2- yl)methyl)amino)pyrimidin-4- yl)cyclopropane-1-carboxamide (I- 191) | 6-chloro-N-((6- cyclopropylimidazo[1,2-a]pyridin-2- yl)methyl)pyrimidin-4-amine ESI-MS (M + H)+: 499.1, ¹H NMR (400 MHz, DMSO) δ 8.74 (d, J = 9.1 Hz, 1H), 8.46 (s, 1H), 8.30 (s, 1H), 8.10 (s, 1H), 8.04 (s, 1H), 7.72 (s, 1H), 7.67 (s, 1H), 7.58 (d, J = 8.8 Hz, 1H), 7.38 (d, J = 9.2 Hz, 1H), 7.04 (s, 1H), 7.02 (s, 1H), 6.96 (d, J = 9.3 Hz, 1H), 4.66 (s, 2H), 2.73- 2.70 (m, 1H), 2.27-2.23 (m, 1H), 1.93- 1.89 (m, 1H), 1.51-1.49 (m, 2H), 0.93-0.88 (m, 2H), 0.66-0.64 (m, 2H). |
| Example 192 | 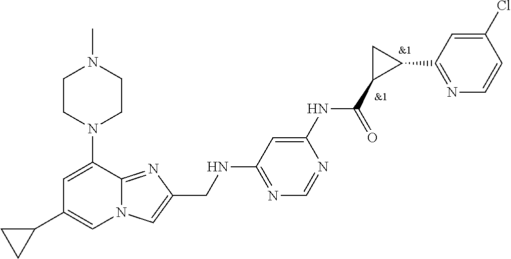 [SMILES: CN1CCN(c2cc(C3CC3)cn3cc(CNc4cc(NC(=O)[C@H]5C[C@@H]5c5cc(Cl)ccn5)ncn4)nc23)CC1] rac-(1S*,2S*)-2-(4-chloropyridin-2- yl)-N-(6-(((6-cyclopropyl-8-(4- methylpiperazin-1-yl)imidazo[1,2- a]pyridin-2- yl)methyl)amino)pyrimidin-4- yl)cyclopropane-1-carboxamide (I- 192) | 6-bromo-N-((6-cyclopropyl-8-(4- methylpiperazin-1-yl)imidazo[1,2- a]pyridin-2-yl)methyl)pyrimidin-4- amine ESI-MS (M + H)+: 558.2, ¹H NMR (400 MHz, DMSO) δ 10.63 (s, 1H), 8.42 (d, J = 5.3 Hz, 1H), 8.18 (s, 1H), 7.86 (s, 2H), 7.64 (s, 1H), 7.51 (s, 1H), 7.38- 7.32 (m, 1H), 7.28 (s, 1H), 6.14 (s, 1H), 4.52 (s, 2H), 3.47 (s, 4H), 3.31 (s, 4H), 2.59-2.58 (m, 2H), 2.23 (s, 3H), 1.88- 1.81 (m, 1H), 1.52-1.47 (m, 2H), 0.88- 0.83 (m, 2H), 0.65-0.64 (m, 2H). |
| Example 193 | 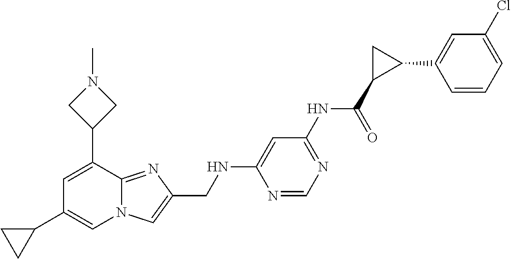 [SMILES: CN1CC(c2cc(C3CC3)cn3cc(CNc4cc(NC(=O)[C@H]5C[C@@H]5c5cccc(Cl)c5)ncn4)nc23)C1] (1S,2S)-2-(3-chlorophenyl)-N-(6-(((6- cyclopropyl-8-(1-methylazetidin-3- yl)imidazo[1,2-a]pyridin-2- yl)methyl)amino)pyrimidin-4- yl)cyclopropane-1-carboxamide (I- 193) | 6-bromo-N-((6-cyclopropyl-8-(1- methylazetidin-3-yl)imidazo[1,2- a]pyridin-2-yl)methyl)pyrimidin-4- amine ESI-MS (M + H)+: 528.2, ¹H NMR (400 MHz, DMSO) δ 10.60 (s, 1H), 8.19 (s, 2H), 7.87 (s, 1H), 7.61 (s, 1H), 7.34- 7.25 (m, 4H), 7.16-7.14 (m, 1H), 6.93 (s, 1H), 4.57 (s, 2H), 4.09-4.01 (m, 1H), 3.90-3.86 (m, 2H), 3.60-3.56 (m, 2H), 2.46 (s, 3H), 2.43-2.36 (m, 2H), 1.93-1.88 (m, 1H), 1.49-1.44 (m, 1H), 1.43-1.38 (m, 1H), 0.93- 0.89 (m, 2H), 0.70-0.66 (m, 2H). |
| Example 194 | 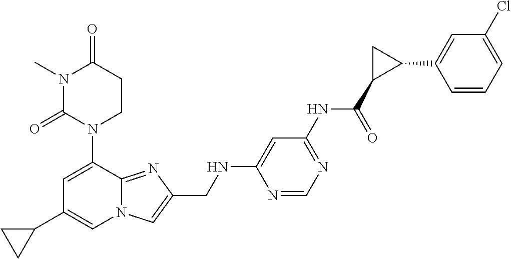 [SMILES: CN1C(=O)CCN(c2cc(C3CC3)cn3cc(CNc4cc(NC(=O)[C@H]5C[C@@H]5c5cccc(Cl)c5)ncn4)nc23)C1=O] (1S,2S)-2-(3-chlorophenyl)-N-(6-(((6- cyclopropyl-8-(3-methyl-2,4- dioxotetrahydropyrimidin-1(2H)- yl)imidazo[1,2-a]pyridin-2- yl)methyl)amino)pyrimidin-4- yl)cyclopropane-1-carboxamide (I- 194) | 1-(2-(((6-bromopyrimidin-4- yl)amino)methyl)-6- cyclopropylimidazo[1,2-a]pyridin-8- yl)-3-methyldihydropyrimidine- 2,4(1H,3H)-dione ESI-MS (M + H)+: 585.2, ¹H NMR (400 MHz, DMSO) δ 10.60 (s, 1H), 8.31 (s, 1H), 8.19 (s, 1H), 7.90 (s, 1H), 7.67 (s, 1H), 7.33-7.25 (m, 4H), 7.15 (d, J = 7.6 Hz, 1H), 7.03 (s, 1H), 4.56 (s, 2H), 3.83 (t, J = 6.6 Hz, 2H), 3.07 (s, 3H), 2.88 (t, J = 6.6 Hz, 2H), 2.42-7.35 (m, 2H), 1.96-1.89 (m, 1H), 1.49-1.38 (m, 2H), 0.95-0.90 (m, 2H), 0.68- 0.64 (m, 2H). |
| Example 195 | 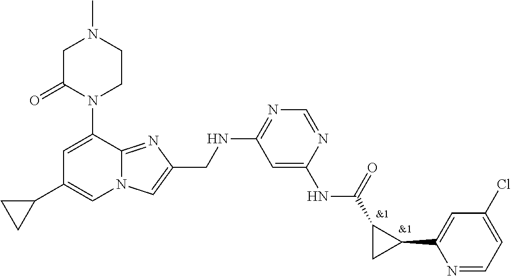 [SMILES: CN1CCN(c2cc(C3CC3)cn3cc(CNc4cc(NC(=O)[C@H]5C[C@@H]5c5cc(Cl)ccn5)ncn4)nc23)C(=O)C1] rac-(1S*,2S*)-2-(4-chloropyridin-2- yl)-N-(6-(((6-cyclopropyl-8-(4-methyl- 2-oxopiperazin-1-yl)imidazo[1,2- a]pyridin-2- yl)methyl)amino)pyrimidin-4- yl)cyclopropane-1-carboxamide (I- 195) | 1-(2-(((6-bromopyrimidin-4- yl)amino)methyl)-6- cyclopropylimidazo[1,2-a]pyridin-8- yl)-4-methylpiperazin-2-one ESI-MS (M + H)+: 572.2, ¹H NMR (400 MHz, DMSO) δ 10.65 (s, 1H), 8.42 (d, J = 5.4 Hz, 1H), 8.29 (s, 1H), 8.18 (s, 1H), 7.92 (s, 1H), 7.64 (s,2H), 7.32-7.26 (m, 1H), 7.28 (s, 1H), 6.94 (d, J = 1.2 Hz, 1H), 4.55 (s, 2H), 3.79-3.70 (m, 2H), 3.15 (s, 2H), 2.79-2.73 ( m, 2H), 2.61-2.52 ( m, 2H), 2.32 (s, 3H), 1.98- 1.86 (m, 1H), 1.58-1.41 (m, 2H), 0.96- 0.86 (m, 2H), 0.72-0.61 (m, 2H). |
| Example 196 | 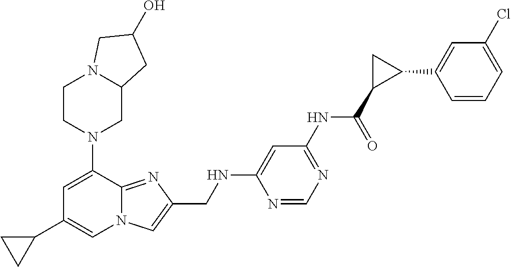 [SMILES: O=C(Nc1cc(NCc2cn3cc(C4CC4)cc(N4CCN5CC(O)CC5C4)c3n2)ncn1)[C@H]1C[C@@H]1c1cccc(Cl)c1] (1S,2S)-2-(3-chlorophenyl)-N-(6-(((6- cyclopropyl-8-(7- hydroxyhexahydropyrrolo[1,2- a]pyrazin-2(1H)-yl)imidazo[1,2- a]pyridin-2- yl)methyl)amino)pyrimidin-4- yl)cyclopropane-1-carboxamide (I- 196) | 2-(2-(((6-bromopyrimidin-4- yl)amino)methyl)-6- cyclopropylimidazo[1,2-a]pyridin-8- yl)octahydropyrrolo[1,2-a]pyrazin-7-ol ESI-MS (M + H)+: 599.2, ¹H NMR (400 MHz, MeOD) δ 8.28 (s, 1H), 8.10 (s, 1H), 7.86 (s, 1H), 7.29-7.04 (m, 6H), 4.81 (s, 2H), 4.76-4.69 (m, 1H), 4.16- 3.71 (m, 4H), 3.69-3.41 (m, 4H), 3.29- 3.10 (m, 2H), 2.74 (s, 1H), 2.53-2.48 (m, 1H), 2.20-2.16 (m, 1H), 2.05- 1.98 (m, 2H), 1.66-1.61 (m, 1H), 1.46- 1.36 (m, 1H), 1.07-1.02 (m, 2H), 0.82-0.78 (m, 2H). |
| Example 197 | 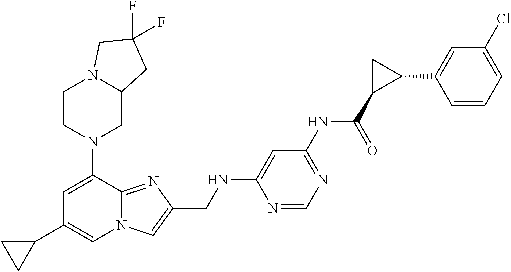 [SMILES: O=C(Nc1cc(NCc2cn3cc(C4CC4)cc(N4CCN5CC(F)(F)CC5C4)c3n2)ncn1)[C@H]1C[C@@H]1c1cccc(Cl)c1] (1S,2S)-2-(3-chlorophenyl)-N-(6-(((6- cyclopropyl-8-(7,7- difluorohexahydropyrrolo[1,2- a]pyrazin-2(1H)-yl)imidazo[1,2- a]pyridin-2- yl)methyl)amino)pyrimidin-4- yl)cyclopropane-1-carboxamide (I- 197) | 6-bromo-N-((6-cyclopropyl-8-(7,7- difluorohexahydropyrrolo[1,2- a]pyrazin-2(1H)-yl)imidazo[1,2- a]pyridin-2-yl)methyl)pyrimidin-4- amine ESI-MS (M + H)+: 619.2, ¹H NMR (400 MHz, DMSO) δ 10.60 (s, 1H), 8.19 (s, 1H), 7.88 (s, 1H), 7.85 (s, 1H), 7.52 (s, 1H), 7.29-7.25 (m, 4H), 7.19-7.14 (m, 1H), 6.18 (s, 1H), 4.54 (s, 2H), 4.41- 4.33 (m, 2H), 3.46-3.41 (m, 2H), 3.03 (d, J = 10.9 Hz, 1H), 2.80 (t, J = 10.7 Hz, 1H), 2.62-2.55 (m, 3H), 2.45- 2.32 (m, 3H), 1.95-1.89 (m, 1H), 1.86-1.82 (m, 1H), 1.48-1.43 (m, 1H), 1.41-1.38 (m, 1H), 0.87-0.84 (m, 2H), 0.67-0.63 (m, 2H). |
| Example 198 | 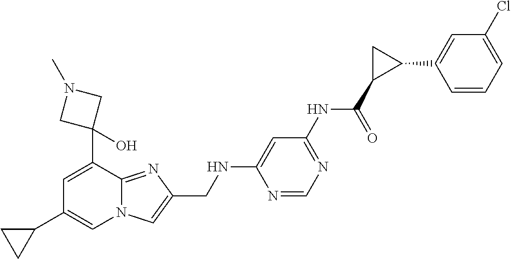 [SMILES: CN1CC(O)(c2cc(C3CC3)cn3cc(CNc4cc(NC(=O)[C@H]5C[C@@H]5c5cccc(Cl)c5)ncn4)nc23)C1] (1S,2S)-2-(3-chlorophenyl)-N-(6-(((6- cyclopropyl-8-(3-hydroxy-1- methylazetidin-3-yl)imidazo[1,2- a]pyridin-2- yl)methyl)amino)pyrimidin-4- yl)cyclopropane-1-carboxamide (I- 198) | 3-(2-(((6-bromopyrimidin-4- yl)amino)methyl)-6- cyclopropylimidazo[1,2-a]pyridin-8- yl)-1-methylazetidin-3-ol ESI-MS (M + H)+: 544.2, ¹H NMR (400 MHz, DMSO) δ 10.61 (s, 1H), 8.24 (d, J = 1.1 Hz, 1H), 8.19 (s, 1H), 7.88 (s, 1H), 7.63 (s, 1H), 7.43-7.35 (m, 1H), 7.35-7.23 (m, 3H), 7.15 (d, J = 7.6 Hz, 1H), 7.09 (d, J = 1.6 Hz, 1H), 6.46 (s, 1H), 4.58 (s, 2H), 3.95 (d, J = 7.2 Hz, 2H), 3.50-3.38 (m, 2H), 2.45-2.28 (m, 5H), 2.00-1.91 (m, 1H), 1.55- 1.43 (m, 1H), 1.43-1.36 (m, 1H), 0.98- 0.87 (m, 2H), 0.69-0.64 (m, 2H). |
| Example 199 | 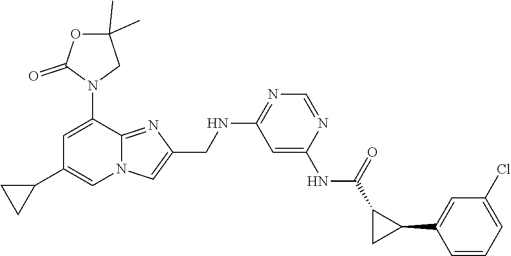 [SMILES: CC1(C)CN(c2cc(C3CC3)cn3cc(CNc4cc(NC(=O)[C@H]5C[C@@H]5c5cccc(Cl)c5)ncn4)nc23)C(=O)O1] (1S,2S)-2-(3-chlorophenyl)-N-(6-(((6- cyclopropyl-8-(5,5-dimethyl-2- oxooxazolidin-3-yl)imidazo[1,2- a]pyridin-2- yl)methyl)amino)pyrimidin-4- yl)cyclopropane-1-carboxamide (I- 199) | 3-(2-(((6-bromopyrimidin-4- yl)amino)methyl)-6- cyclopropylimidazo[1,2-a]pyridin-8- yl)-5,5-dimethyloxazolidin-2-one ESI-MS (M + H)+: 572.2, ¹H NMR (400 MHz, DMSO) δ 10.61 (s, 1H), 8.33- 8.13 (m, 2H), 7.88 (s, 1H), 7.66 (s, 1H), 7.39-7.22 (m, 5H), 7.15 (d, J = 7.6 Hz, 1H), 4.57 (s, 2H), 4.23 (s, 2H), 2.44- 2.33 (m, 2H), 2.01-1.85 (m, 1H), 1.51 (s, 6H), 1.49-1.37 (m, 2H), 0.98-0.86 (m, 2H), 0.72-0.55 (m, 2H). |
| Example 200 | 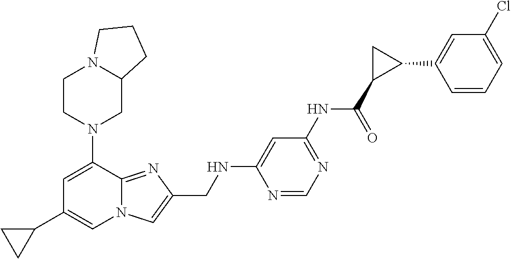 [SMILES: O=C(Nc1cc(NCc2cn3cc(C4CC4)cc(N4CCN5CCCC5C4)c3n2)ncn1)[C@H]1C[C@@H]1c1cccc(Cl)c1] (1S,2S)-2-(3-chlorophenyl)-N-(6-(((6- cyclopropyl-8-(hexahydropyrrolo[1,2- a]pyrazin-2(1H)-yl)imidazo[1,2- a]pyridin-2- yl)methyl)amino)pyrimidin-4- yl)cyclopropane-1-carboxamide (I- 200) | 6-bromo-N-((6-cyclopropyl-8- (hexahydropyrrolo[1,2-a]pyrazin- 2(1H)-yl)imidazo[1,2-a]pyridin-2- yl)methyl)pyrimidin-4-amine ESI-MS (M + H)+: 583.1, ¹H NMR (400 MHz, cd3od) δ 8.17 (s, 1H), 7.78 (s, 1H), 7.55 (s, 1H), 7.33 (s, 1H), 7.26- 7.16 (m, 3H), 7.08 (d, J = 7.6 Hz, 1H), 6.41 (s, 1H), 4.68 (S, J = 36.7 Hz, 2H), 3.01-2.82 (m, 3H), 2.70-2.43 (m, 3H), 2.20-1.85 (m, 6H), 1.65-1.56 (m, 2H), 1.39-1.25 (m, 4H), 0.94- 0.87 (m, 2H), 0.70-0.66 (m, 2H). |
| Example 201 | 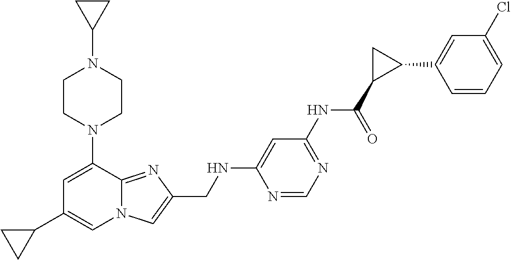 [SMILES: O=C(Nc1cc(NCc2cn3cc(C4CC4)cc(N4CCN(C5CC5)CC4)c3n2)ncn1)[C@H]1C[C@@H]1c1cccc(Cl)c1] (1S,2S)-2-(3-chlorophenyl)-N-(6-(((6- cyclopropyl-8-(4- cyclopropylpiperazin-1- yl)imidazo[1,2-a]pyridin-2- yl)methyl)amino)pyrimidin-4- yl)cyclopropane-1-carboxamide (I- 201) | 6-bromo-N-((6-cyclopropyl-8-(4- cyclopropylpiperazin-1-yl)imidazo[1,2- a]pyridin-2-yl)methyl)pyrimidin-4- amine ESI-MS (M + H)+: 583.3, ¹H NMR (400 MHz, DMSO) δ 10.58 (s, 1H), 8.17 (s, 1H), 7.85 (s, 2H), 7.50 (s, 1H), 7.31- 7.23 (m, 4H), 7.12 (d, J = 7.6 Hz, 1H), 6.12 (s, 1H), 4.52 (s, 2H), 3.39 (s, 4H), 2.67 (s, 4H), 2.41-2.31 (m, 2H), 1.84- 1.80 (m, 1H), 1.66 (s, 1H), 1.47-1.40 (m, 1H), 1.40-1.36 (m, 1H), 0.86- 0.81 (m, 2H), 0.66-0.62 (m, 2H), 0.43- 0.32 (m, 4H). |
| Example 202 | 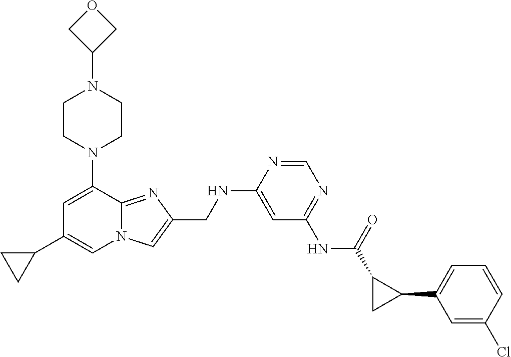 [SMILES: O=C(Nc1cc(NCc2cn3cc(C4CC4)cc(N4CCN(C5COC5)CC4)c3n2)ncn1)[C@H]1C[C@@H]1c1cccc(Cl)c1] (1S,2S)-2-(3-chlorophenyl)-N-(6-(((6- cyclopropyl-8-(4-(oxetan-3- yl)piperazin-1-yl)imidazo[1,2- a]pyridin-2- yl)methyl)amino)pyrimidin-4- yl)cyclopropane-1-carboxamide (I- 202) | 6-bromo-N-((6-cyclopropyl-8-(4- (oxetan-3-yl)piperazin-1- yl)imidazo[1,2-a]pyridin-2- yl)methyl)pyrimidin-4-amine ESI-MS (M + H)+: 599.2, ¹H NMR (400 MHz, DMSO) δ 10.59 (s, 1H), 8.18 (s, 1H), 7.88-7.86 (m, 2H), 7.51 (s,1H), 7.34-7.23 (m, 4H), 7.15 (d, J = 7.6 Hz, 1H), 6.15 (s, 1H), 4.59-4.46 (m, 6H), 3.50-3.42 (m, 5H), 2.40-2.32 (m, 6H), 1.88-1.80 (m, 1H), 1.46-1.42 (m, 2H), 0.91-0.82 (m, 2H), 0.68-0.59 (m, 2H). |
| Example 203 | 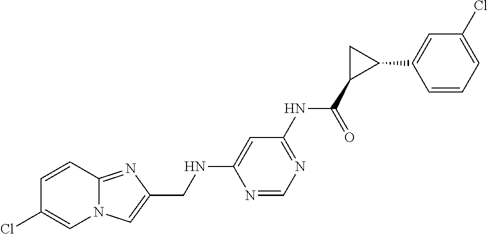 [SMILES: O=C(Nc1cc(NCc2cn3cc(Cl)ccc3n2)ncn1)[C@H]1C[C@@H]1c1cccc(Cl)c1] (1S,2S)-N-(6-(((6-chloroimidazo[1,2- a]pyridin-2- yl)methyl)amino)pyrimidin-4-yl)-2-(3- chlorophenyl)cyclopropane-1- carboxamide (I-203) | 6-bromo-N-((6-chloroimidazo[1,2- a]pyridin-2-yl)methyl)pyrimidin-4- amine ESI-MS (M + H)+: 453.1, ¹H NMR (400 MHz, DMSO) δ 10.61 (s, 1H), 8.77 (d, J = 1.4 Hz, 1H), 8.20 (s, 1H), 7.92 (s, 1H), 7.73 (s, 1H), 7.53 (d, J = 9.6 Hz, 1H), 7.34-7.24 (m, 5H), 7.15 (d, J = 7.6 Hz, 1H), 4.60 (s, 2H), 2.43-2.36 (m, 2H), 1.49-1.45 (m, 1H), 1.43-1.39 (m, 1H). |
| Example 204 | 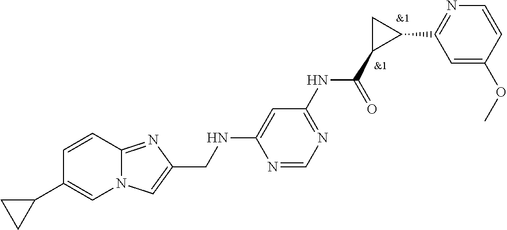 [SMILES: COc1ccnc([C@H]2C[C@@H]2C(=O)Nc2cc(NCc3cn4cc(C5CC5)ccc4n3)ncn2)c1] rac-(1S*,2S*)-N-(6-(((6- cyclopropylimidazo[1,2-a]pyridin-2- yl)methyl)amino)pyrimidin-4-yl)-2-(4- methoxypyridin-2-yl)cyclopropane-1- carboxamide (I-204) | 6-bromo-N-((6- cyclopropylimidazo[1,2-a]pyridin-2- yl)methyl)pyrimidin-4-amine ESI-MS (M + H)+: 456.2, ¹H NMR (400 MHz, CDCl₃) δ 8.31-8.24 (m, 2H), 8.06 (s, 1H), 7.88 (s, 1H), 7.51-7.46 (m, 2H), 7.33 (s, 1H), 6.99 (d, J = 10.7 Hz, 1H), 6.81 (d, J = 2.4 Hz, 1H), 6.69-6.64 (m, 1H), 5.80 (s, 1H), 4.71 (s, 2H), 3.85 (s, 3H), 2.63-2.56 (m, 1H), 2.25-2.18 (m, 1H), 1.92-1.89 (m, 2H), 1.30-1.22 (m, 1H), 1.03-0.94 (m, 2H), 0.72- 0.65 (m, 2H). |
| Example 205 | 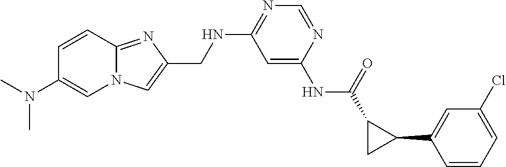 [SMILES: CN(C)c1ccc2nc(CNc3cc(NC(=O)[C@H]4C[C@@H]4c4cccc(Cl)c4)ncn3)cn2c1] (1S,2S)-2-(3-chlorophenyl)-N-(6-(((6- (dimethylamino)imidazo[1,2-a]pyridin- 2-yl)methyl)amino)pyrimidin-4- yl)cyclopropane-1-carboxamide (I- 205) | 2-(((6-chloropyrimidin-4- yl)amino)methyl)-N,N- dimethylimidazo[1,2-a]pyridin-6-amine ESI-MS (M + H)+: 462.1, ¹H NMR (400 MHz, DMSO) δ 10.58 (s, 1H), 8.19 (s, 1H), 7.87-7.76 (m, 2H), 7.57 (s, 1H), 7.35-7.22 (m, 5H), 7.16-7.13 (m, 2H), 4.53 (s, 2H), 2.78 (s, 6H), 2.43- 2.36 (m, 2H), 1.48-1.43 (m, 1H), 1.42- 1.36 (m, 1H). ESI-MS [M +H]+:: 462.1 |
| Example 206 | 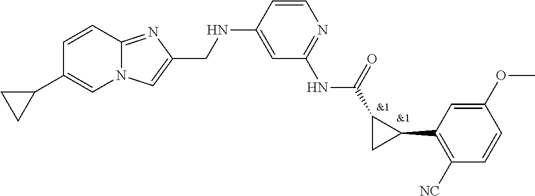 [SMILES: COc1ccc(C#N)c([C@H]2C[C@@H]2C(=O)Nc2cc(NCc3cn4cc(C5CC5)ccc4n3)ccn2)c1] rac-(1S*,2S*)-2-(2-cyano-5- methoxyphenyl)-N-(4-(((6- cyclopropylimidazo[1,2-a]pyridin-2- yl)methyl)amino)pyridin-2- yl)cyclopropane-1-carboxamide (I- 206) | 2-bromo-N-((6- cyclopropylimidazo[1,2-a]pyridin-2- yl)methyl)pyridin-4-amine ESI-MS (M + H)+: 480.2, ¹H NMR (400 MHz, DMSO) δ 10.66 (s, 1H), 8.29 (s, 1H), 8.20 (s, 1H), 7.88 (s, 1H), 7.74 (d, J = 8.6 Hz, 1H), 7.61 (s, 1H), 7.37 (d, J = 9.3 Hz, 1H), 7.29 (s, 1H), 6.94-6.99 (m, 2H), 6.76 (d, J = 2.4 Hz, 1H), 4.58 (s, 2H), 3.84 (s, 3H), 2.60-2.53 (m, 1H), 2.49-2.40 (m, 1H), 1.94-1.87 (m, 1H), 1.56-1.51 (m, 2H), 0.98-0.87 (m, 2H), 0.66-0.60 (m, 2H). |
| Example 207 | 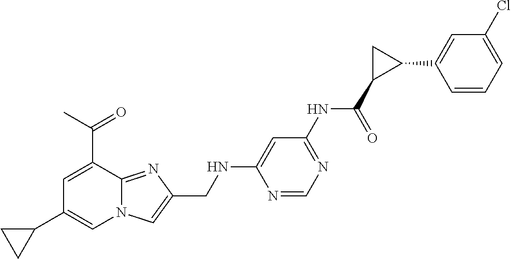 [SMILES: CC(=O)c1cc(C2CC2)cn2cc(CNc3cc(NC(=O)[C@H]4C[C@@H]4c4cccc(Cl)c4)ncn3)nc12] (1S,2S)-N-(6-(((8-acetyl-6- cyclopropylimidazo[1,2-a]pyridin-2- yl)methyl)amino)pyrimidin-4-yl)-2-(3- chlorophenyl)cyclopropane-1- carboxamide (I-207) | 1-(2-(((2-bromopyridin-4- yl)amino)methyl)-6- cyclopropylimidazo[1,2-a]pyridin-8- yl)ethan-1-one ESI-MS (M + H)+: 501.2, ¹H NMR (400 MHz, CDCl₃) δ 8.29 (s, 1H), δ 8.25 (s, 1H), 8.04 (s, 1H), 7.62 (s, 1H), 7.53 (s, 1H), 7.36 (s, 1H), 7.24-7.21 (m, 2H), 7.08 (s, 1H), 7.00 (d, J = 6.1 Hz, 1H), 5.85 (s, 1H), 4.72 (s, 2H), 3.03 (s, 3H), 2.61-2.56 (m, 1H), 1.94-1.89 (m, 1H), 1.77-1.71 (m, 2H), 1.42-1.38 (m, 1H), 1.02-0.99 (m, 2H), 0.74- 0.71 (m, 2H). ESI-MS [M + H]+: 501.2. |
| Example 208 | 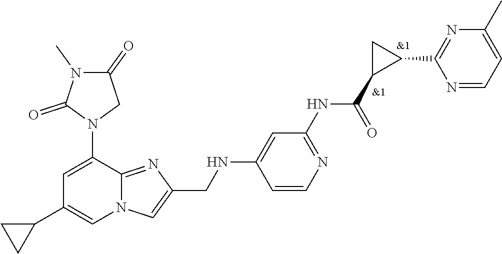 [SMILES: Cc1ccnc([C@H]2C[C@@H]2C(=O)Nc2cc(NCc3cn4cc(C5CC5)cc(N5CC(=O)N(C)C5=O)c4n3)ccn2)n1] rac-(1S*,2S*)-N-(4-(((6-cyclopropyl- 8-(3-methyl-2,4-dioxoimidazolidin-1- yl)imidazo[1,2-a]pyridin-2- yl)methyl)amino)pyridin-2-yl)-2-(4- methylpyrimidin-2-yl)cyclopropane-1- carboxamide (I-208) | 1-(2-(((2-bromopyridin-4- yl)amino)methyl)-6- cyclopropylimidazo[1,2-a]pyridin-8- yl)-3-methylimidazolidine-2,4-dione ESI-MS [M + H]+: 552.2. ¹H NMR (400 MHz, DMSO) δ 10.39 (s, 1H), 8.52 (d, J = 5.1 Hz, 1H), 8.29-8.26 (m, 2H), 7.75 (d, J = 5.8 Hz, 1H), 7.71 (s, 1H), 7.46 (s, 1H), 7.34 (d, J = 1.4 Hz, 1H), 7.20 (d, J = 5.1 Hz, 1H), 7.15 (t, J = 5.7 Hz, 1H), 6.32 (dd, J = 5.7, 2.0 Hz, 1H), 4.93 (s, 2H), 4.37 (d, J = 5.6 Hz, 2H), 2.98 (s, 3H), 2.62-2.52 (m, 2H), 2.41 (s, 3H), 1.97-1.90 (m, 1H), 1.52-1.44 (m, 2H), 1.02-0.85 (m, 2H), 0.71- 0.53 (m, 2H). |
| Example 209 | 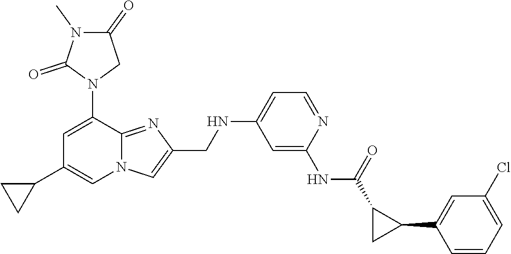 [SMILES: CN1C(=O)CN(c2cc(C3CC3)cn3cc(CNc4ccnc(NC(=O)[C@H]5C[C@@H]5c5cccc(Cl)c5)c4)nc23)C1=O] (1S,2S)-2-(3-chlorophenyl)-N-(4-(((6- cyclopropyl-8-(3-methyl-2,4- dioxoimidazolidin-1-yl)imidazo[1,2- a]pyridin-2-yl)methyl)amino)pyridin- 2-yl)cyclopropane-1-carboxamide (I- 209) | 1-(2-(((2-bromopyridin-4- yl)amino)methyl)-6- cyclopropylimidazo[1,2-a]pyridin-8- yl)-3-methylimidazolidine-2,4-dione ESI-MS [M + H]+: 570.2. ¹H NMR (400 MHz, DMSO) δ 10.31 (s, 1H), 8.23 (d, J = 1.1 Hz, 1H), 7.73 (d, J = 5.8 Hz, 1H), 7.68 (s, 1H), 7.43 (s, 1H), 7.36-7.16 (m, 5H), 7.11 (d, J = 7.7 Hz, 1H), 6.30 (d, J = 4.2 Hz, 1H), 4.90 (s, 2H), 4.35 (d, J = 5.6 Hz, 2H), 2.95 (s, 3H), 2.37-2.28 (m, 2H), 1.94-1.88 (m, 1H), 1.45- 1.38 (m, 1H), 1.35-1.28 (m, 1H), 0.95- 0.87 (m, 2H), 0.66-0.59 (m, 2H). |
| Example 210 | 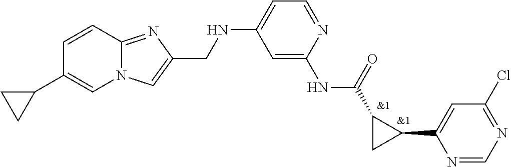 [SMILES: O=C(Nc1cc(NCc2cn3cc(C4CC4)ccc3n2)ccn1)[C@H]1C[C@@H]1c1cc(Cl)ncn1] rac-(1S*,2S*)-2-(6-chloropyrimidin-4- yl)-N-(4-(((6-cyclopropylimidazo[1,2- a]pyridin-2-yl)methyl)amino)pyridin- 2-yl)cyclopropane-1-carboxamide (I- 210) | 2-bromo-N-((6- cyclopropylimidazo[1,2-a]pyridin-2- yl)methyl)pyridin-4-amine ESI-MS [M + H]+: 460.2., ¹H NMR (400 MHz, DMSO) δ 10.42 (s, 1H), 8.88 (s, 1H), 8.31 (s, 1H), 7.85 (s, 1H), 7.75 (d, J = 5.4 Hz, 1H), 7.62 (s, 1H), 7.41 (s, 1H), 7.38 (d, J = 9.6 Hz, 1H), 7.12 (s, 1H), 6.97 (d, J = 8.9 Hz, 1H), 6.33 (d, J = 4.4 Hz, 1H), 4.34 (d, J = 5.6 Hz, 2H), 2.72-2.64 (m, 2H), 2.04-1.95 (m, 1H), 1.94-1.87 (m, 1H), 1.60-1.51 (m, 1H), 0.95-0.86 (m, 2H), 0.69- 0.62 (m, 2H). |
| Example 211 | 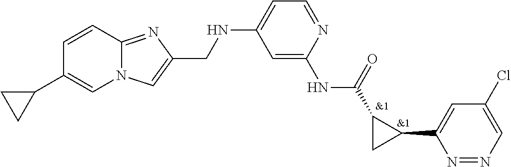 [SMILES: O=C(Nc1cc(NCc2cn3cc(C4CC4)ccc3n2)ccn1)[C@H]1C[C@@H]1c1cc(Cl)cnn1] rac-(1S*,2S*)-2-(5-chloropyridazin-3- yl)-N-(4-(((6-cyclopropylimidazo[1,2- a]pyridin-2-yl)methyl)amino)pyridin- 2-yl)cyclopropane-1-carboxamide (I- 211) | 2-bromo-N-((6- cyclopropylimidazo[1,2-a]pyridin-2- yl)methyl)pyridin-4-amine ESI-MS [M + H]+: 460.2, ¹H NMR (400 MHz, DMSO) δ 10.41 (s, 1H), 9.23 (d, J = 2.1 Hz, 1H), 8.31 (s, 1H), 8.02 (d, J = 2.2 Hz, 1H), 7.76 (d, J = 5.7 Hz, 1H), 7.63 (s, 1H), 7.43 (s, 1H), 7.38 (d, J = 9.3 Hz, 1H), 7.13 (t, J = 2.2 Hz , 1H), 7.01-6.96 (m, 1H), 6.39-6.33 (m, 1H), 4.35 (d, J = 5.6 Hz, 2H), 2.76-2.62 (m, 2H), 1.96-1.85 (m, 1H), 1.70-1.64 (m, 1H), 1.62-1.49 (m, 1H), 0.91- 0.85 (m, 2H), 0.68-0.64 (m, 2H). |
| Example 212 | 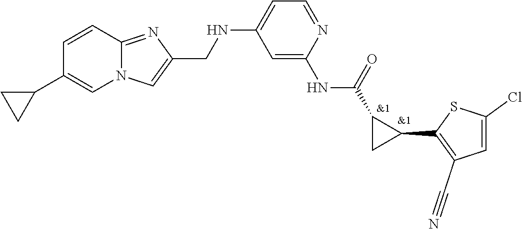 [SMILES: N#Cc1cc(Cl)sc1[C@H]1C[C@@H]1C(=O)Nc1cc(NCc2cn3cc(C4CC4)ccc3n2)ccn1] rac-(1S*,2S*)-2-(5-chloro-3- cyanothiophen-2-yl)-N-(4-(((6- cyclopropylimidazo[1,2-a]pyridin-2- yl)methyl)amino)pyridin-2- yl)cyclopropane-1-carboxamide (I- 212) | 2-bromo-N-((6- cyclopropylimidazo[1,2-a]pyridin-2- yl)methyl)pyridin-4-amine ESI-MS [M + H]+: 489.1, ¹H NMR (400 MHz, DMSO) δ 10.52 (s, 1H), 8.30 (s, 1H), 7.77 (d, J = 5.8 Hz, 1H), 7.63 (s, 1H), 7.51 (s, 1H), 7.42 (s, 1H), 7.38 (d, J = 9.3 Hz, 1H), 7.15 (s, 1H), 6.98-6.95 (m, 1H), 6.35-6.33 (m, 1H), 4.35 (d, J = 5.7 Hz, 2H), 2.70-2.63 (m, 2H), 1.94- 1.87 (m, 1H), 1.65-1.60 (m, 1H), 1.44- 1.37 (m, 1H), 0.93-0.88 (m, 2H), 0.68- 0.64 (m, 2H). |
| Example 213 | 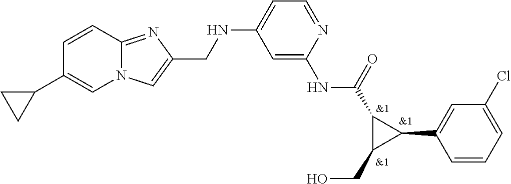 [SMILES: O=C(Nc1cc(NCc2cn3cc(C4CC4)ccc3n2)ccn1)[C@H]1[C@H](CO)[C@@H]1c1cccc(Cl)c1] rac-(1R*,2S*,3R*)-2-(3- chlorophenyl)-N-(4-(((6- cyclopropylimidazo[1,2-a]pyridin-2- yl)methyl)amino)pyridin-2-yl)-3- (hydroxymethyl)cyclopropane-1- carboxamide (I-213) | 2-bromo-N-((6- cyclopropylimidazo[1,2-a]pyridin-2- yl)methyl)pyridin-4-amine ESI-MS [M + H]+: 488.2, ¹H NMR (400 MHz, DMSO) δ 10.64 (s, 1H), 8.29 (s, 1H), 8.22 (s, 1H), 7.86 (s, 1H), 7.61 (s, 11H), 7.46-7.27 (m, 6H), 6.97-6.94 (m, 1H), 4.65-4.56 (m, 1H), 4.57 (s, 2H), 3.22-3.15 (m, 1H), 3.14-3.02 (m, 1H), 2.63-2.60 (m, 1H), 2.57-2.50 (m, 1H), 1.90-1.85 (m, 2H), 0.92-0.85 (m, 2H), 0.67-0.64(m, 2H). |
| Example 214 | 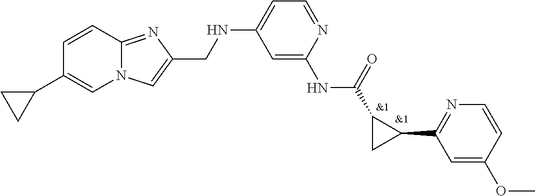 rac-(1S*,2S*)-N-(4-(((6- cyclopropylimidazo[1,2-a]pyridin-2- yl)methyl)amino)pyridin-2-yl)-2-(4- methoxypyridin-2-yl)cyclopropane-1- carboxamide (I-214) | 2-bromo-N-((6- cyclopropylimidazo[1,2-a]pyridin-2- yl)methyl)pyridin-4-amine ESI-MS [M + H]+: 555.1, ¹H NMR (400 MHz, CDCl₃) δ 8.58 (s, 1H), 8.25 (d, J = 5.7 Hz, 1H), 7.86 (s, 1H), 7.79 (d, J = 5.9 Hz, 1H), 7.60 (s, 1H), 7.48-7.41 (m, 2H), 6.94 (dd, J = 9.3, 1.7 Hz, 1H), 6.79 (d, J = 2.4 Hz, 1H), 6.64 (dd, J = 5.7, 2.5 Hz, 1H), 6.27 (dd, J = 5.9, 2.2 Hz, 1H), 5.18 (s, 1H), 4.52 (d, J = 5.2 Hz, 2H), 3.84 (s, 3H), 2.63-2.55 (m, 1H), 2.26-2.18 (m, 1H), 1.92-1.86 (m, 1H), 1.69-1.61 (m, 2H), 0.96- 0.94 (m, 2H), 0.71-0.64 (m, 2H). |
| Example 215 | 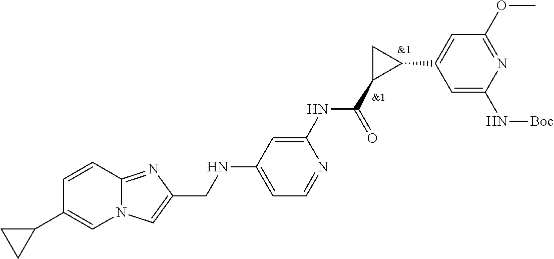 [SMILES: COc1cc([C@H]2C[C@@H]2C(=O)Nc2cc(NCc3cn4cc(C5CC5)ccc4n3)ccn2)cc(NC(=O)OC(C)(C)C)n1] rac-tert-butyl (4-((1S*,2S*)-2-((4-(((6- cyclopropylimidazo[1,2-a]pyridin-2- yl)methyl)amino)pyridin-2- yl)carbamoyl)cyclopropyl)-6- methoxypyridin-2-yl)carbamate (I-216) | 2-bromo-N-((6- cyclopropylimidazo[1,2-a]pyridin-2- yl)methyl)pyridin-4-amine ESI-MS [M + H]+: 570.2. ¹H NMR (400 MHz, MeOD) δ 8.17 (s, 1H), 7.78 (d, J = 6.1 Hz, 1H), 7.65 (s, 1H), 7.40 (d, J = 9.4 Hz, 1H), 7.32 (s, 1H), 7.21 (s, 1H), 7.09 (dd, J = 9.4, 1.7 Hz, 1H), 6.42 (dd, J = 6.1, 2.2 Hz, 1H), 6.26 (s, 1H), 4.51 (s, 2H), 3.84 (s, 3H), 2.44-2.39 (m, 1H), 2.30-2.24 (m, 1H), 1.98-1.92 (m, 1H), 1.66-1.61 (m, 1H), 1.54 (s, 9H), 1.44-1.39 (m, 1H), 1.01-0.95 (m, 2H), 0.75-0.70 (m, 2H). |
| Example 217 | 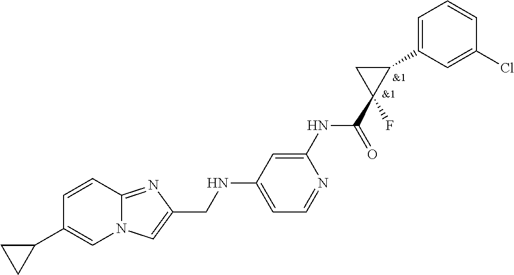 [SMILES: O=C(Nc1cc(NCc2cn3cc(C4CC4)ccc3n2)ccn1)[C@@]1(F)C[C@@H]1c1cccc(Cl)c1] rac-(1S*,2S*)-2-(3-chlorophenyl)-N- (4-(((6-cyclopropylimidazo[1,2- a]pyridin-2-yl)methyl)amino)pyridin- 2-yl)-1-fluorocyclopropane-1- carboxamide (I-217) | 2-bromo-N-((6- cyclopropylimidazo[1,2-a]pyridin-2- yl)methyl)pyridin-4-amine ESI-MS [M + H]+: 476.2, ¹H NMR (400 MHz, DMSO) δ 9.69 (s, 1H), 8.32 (s, 1H), 7.81 (d, J = 5.8 Hz, 1H), 7.65 (s, 1H), 7.44 (s, 1H), 7.42-7.27 (m, 5H), 7.29-7.21 (m, 1H), 6.98-6.96 (m, 1H), 6.43-6.41 (m, 1H), 4.38 (d, J = 5.5 Hz, 2H), 2.90-2.85(m, 1H), 2.16-2.02 (m, 1H), 1.93-1.82 (m, 2H), 0.92-0.89(m, 2H), 0.72-0.64 (m, 2H). |
| Example 218 | 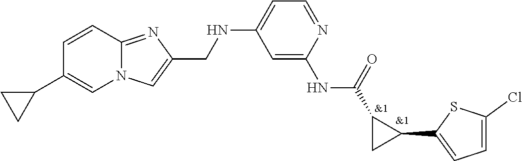 [SMILES: O=C(Nc1cc(NCc2cn3cc(C4CC4)ccc3n2)ccn1)[C@H]1C[C@@H]1c1ccc(Cl)s1] rac-(1S*,2S*)-2-(5-chlorothiophen-2- yl)-N-(4-(((6-cyclopropylimidazo[1,2- a]pyridin-2-yl)methyl)amino)pyridin- 2-yl)cyclopropane-1-carboxamide (I- 218) | 2-bromo-N-((6- cyclopropylimidazo[1,2-a]pyridin-2- yl)methyl)pyridin-4-amine ESI-MS [M + H]+: 464.2, ¹H NMR (400 MHz, DMSO) δ = 9.69 (s, 1H), 8.32 (s, 1H), 7.81 (d, J = 5.8, 1H), 7.65 (s, 1H), 7.42 (d, J = 16.4, 1H), 7.38-7.31 (m, 3H), 7.29-7.20 (m, 1H), 7.03-6.90 (m, 1H), 6.48-6.36 (m, 1H), 4.38 (d, J = 5.5, 2H), 3.31 (s, 1H), 2.97-2.81 (m, 1H), 2.17-2.03 (m, 1H), 1.96-1.76 (m, 2H), 1.01-0.83 (m, 2H), 0.73-0.55 (m, 2H). |
| Example 219 | 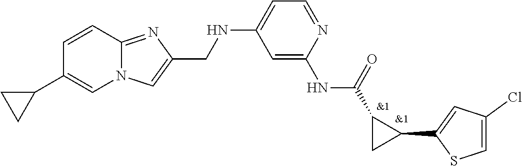 [SMILES: O=C(Nc1cc(NCc2cn3cc(C4CC4)ccc3n2)ccn1)[C@H]1C[C@@H]1c1cc(Cl)cs1] rac-(1S*,2S*)-2-(4-chlorothiophen-2- yl)-N-(4-(((6-cyclopropylimidazo[1,2- a]pyridin-2-yl)methyl)amino)pyridin- 2-yl)cyclopropane-1-carboxamide (I- 219) | 2-bromo-N-((6- cyclopropylimidazo[1,2-a]pyridin-2- yl)methyl)pyridin-4-amine ESI-MS [M + H]+: 464.1, ¹H NMR (400 MHz, DMSO) δ = 10.40 (s, 1H), 8.31 (s, 1H), 7.76 (d, J = 5.8, 1H), 7.62 (s, 1H), 7.45-7.31 (m, 3H), 7.12 (s, 1H), 7.00- 6.91 (m, 2H), 6.36-6.30 (m, 1H), 4.35 (d, J = 5.4, 2H), 2.52-2.50 (m, 1H), 2.35- 2.33 (m, 1H), 1.94-1.83 (m, 1H), 1.50-1.40 (m, 1H), 1.33-1.25 (m, 1H), 0.94-0.85 (m, 2H), 0.72-0.62 (m, 2H). |
| Example 220 | 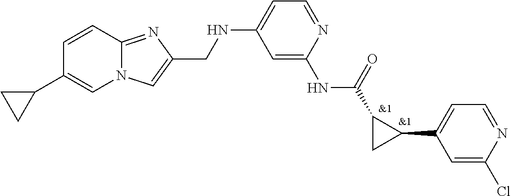 [SMILES: O=C(Nc1cc(NCc2cn3cc(C4CC4)ccc3n2)ccn1)[C@H]1C[C@@H]1c1ccnc(Cl)c1] rac-(1S*,2S*)-2-(2-chloropyridin-4- yl)-N-(4-(((6-cyclopropylimidazo[1,2- a]pyridin-2-yl)methyl)amino)pyridin- 2-yl)cyclopropane-1-carboxamide (I- 220) | 2-bromo-N-((6- cyclopropylimidazo[1,2-a]pyridin-2- yl)methyl)pyridin-4-amine ESI-MS [M + H]+: 459.2, ¹H NMR (400 MHz, DMSO) δ 10.37 (s, 0.62H), 10.03 (s, 0.32H), 8.31-8.22 (m, 2H), 7.76- 7.74 (m, 1H), 7.61 (d, J = 8.1 Hz, 1H), 7.47-7.33 (m, 3H), 7.26-7.07 (m, 2H), 7.01-6.90 (m, 1H), 6.35-6.31 (m, 1H), 4.36-4.32 (m , 2H), 2.49-2.35 (m, 1H), 2.05-1.84 (m, 1H), 1.50- 1.33 (m, 2H), 1.07-0.88 (m, 2H), 0.67-0.63 (m, 2H). |
| Example 221 | 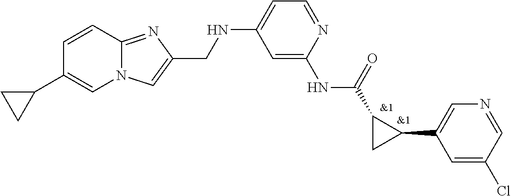 [SMILES: O=C(Nc1cc(NCc2cn3cc(C4CC4)ccc3n2)ccn1)[C@H]1C[C@@H]1c1cncc(Cl)c1] rac-(1S*,2S*)-2-(5-chloropyridin-3- yl)-N-(4-(((6-cyclopropylimidazo[1,2- a]pyridin-2-yl)methyl)amino)pyridin- 2-yl)cyclopropane-1-carboxamide (I- 221) | 2-bromo-N-((6- cyclopropylimidazo[1,2-a]pyridin-2- yl)methyl)pyridin-4-amine ESI-MS [M + H]+: 459.2, ¹H NMR (400 MHz, DMSO) δ 10.33 (s, 1H), 8.47- 8.41 (m, 2H), 8.31 (s, 1H), 7.76 (d, J = 5.8 Hz, 1H), 7.75-7.69 (m, 1H), 7.63 (s, 1H), 7.41 (s, 1H), 7.38 (d, J = 9.3 Hz, 1H), 7.13 (s, 1H), 6.98-6.95 (m, 1H), 6.34-6.32 (m, 1H), 4.35 (d, J = 5.7 Hz, 2H), 2.47-2.38 (m, 2H), 1.96-1.85 (m, 1H), 1.54-1.42 (m, 2H), 0.94-0.82 (m, 2H), 0.72-0.60 (m, 2H). |
| Example 222 | 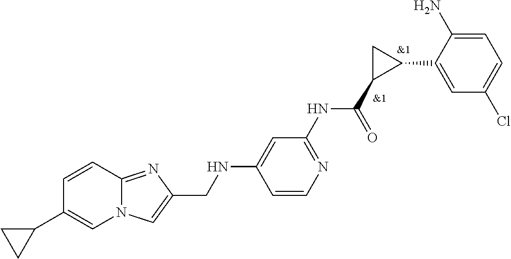 [SMILES: Nc1ccc(Cl)cc1[C@H]1C[C@@H]1C(=O)Nc1cc(NCc2cn3cc(C4CC4)ccc3n2)ccn1] rac-(1S*,2S*)-2-(2-amino-5- chlorophenyl)-N-(4-(((6- cyclopropylimidazo[1,2-a]pyridin-2- yl)methyl)amino)pyridin-2- yl)cyclopropane-1-carboxamide (I- 222) | 2-bromo-N-((6- cyclopropylimidazo[1,2-a]pyridin-2- yl)methyl)pyridin-4-amine ESI-MS [M + H]+: 473.3. ¹H NMR (400 MHz, DMSO) δ 10.40 (s, 1H), 8.30 (s, 1H), 7.77 (d, J = 5.8 Hz, 1H), 7.63 (s, 1H), 7.45 (s, 1H), 7.38 (d, J = 9.3 Hz, 1H), 7.15-7.06 (m, 1H), 6.95-6.91 (m, 3H), 6.66- 6.60 (m, 1H), 6.33 (dd, J = 5.8, 2.1 Hz, 1H), 5.12-5.05 (m, 2H), 4.35 (d, J = 5.5 Hz, 2H), 2.29-2.24 (m, 1H), 2.14-2.09 (m, 1H), 1.94-1.87 (m, 1H), 1.44-1.36 (m, 1H), 1.05- 0.97 (m, 1H), 0.92-0.85 (m, 2H), 0.68- 0.64 (m, 2H). |
| Example 223 | 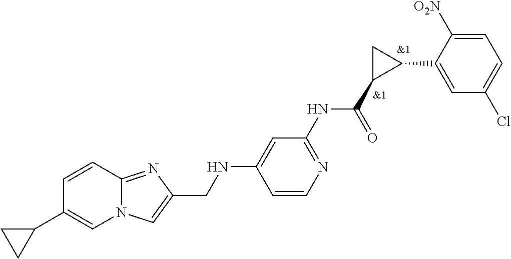 [SMILES: O=C(Nc1cc(NCc2cn3cc(C4CC4)ccc3n2)ccn1)[C@H]1C[C@@H]1c1cc(Cl)ccc1[N+](=O)[O-]] rac-(1S*,2S*)-2-(5-chloro-2- nitrophenyl)-N-(4-(((6- cyclopropylimidazo[1,2-a]pyridin-2- yl)methyl)amino)pyridin-2- yl)cyclopropane-1-carboxamide (I- 223) | 2-bromo-N-((6- cyclopropylimidazo[1,2-a]pyridin-2- yl)methyl)pyridin-4-amine ESI-MS [M + H]+: 503.1, ¹H NMR (400 MHz, DMSO) δ 10.36 (s, 1H), 8.31 (s, 1H), 8.02 (d, J = 8.7 Hz, 1H), 7.77 (d, J = 5.8 Hz, 1H), 7.64 (s, 1H), 7.59 (dd, J = 8.7, 2.2 Hz, 1H), 7.55-7.54 (m, 1H), 7.44 (s, 1H), 7.38 (d, J = 9.3 Hz, 1H), 7.15 (s, 1H), 6.97 (dd, J = 9.3, 1.8 Hz, 1H), 6.33 (dd, J = 5.8, 2.0 Hz, 1H), 4.36 (d, J = 5.7 Hz, 2H), 2.69-2.64 (m, 1H), 2.39-2.33 (m, 1H), 1.90-1.87 (m, 1H), 1.48-1.42 (m, 2H), 0.89-0.88 (m, 2H), 0.68-0.64 (m, 2H). |
| Example 224 | 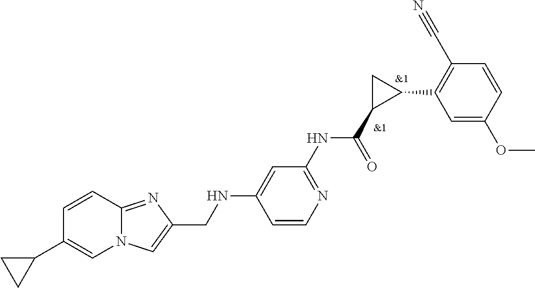 [SMILES: COc1ccc(C#N)c([C@H]2C[C@@H]2C(=O)Nc2cc(NCc3cn4cc(C5CC5)ccc4n3)ccn2)c1] rac-(1S*,2S*)-2-(2-cyano-5- methoxyphenyl)-N-(4-(((6- cyclopropylimidazo[1,2-a]pyridin-2- yl)methyl)amino)pyridin-2- yl)cyclopropane-1-carboxamide (I- 224) | 2-bromo-N-((6- cyclopropylimidazo[1,2-a]pyridin-2- yl)methyl)pyridin-4-amine ESI-MS [M + H]+: 479.2, ¹H NMR (400 MHz, DMSO) δ 10.39 (s, 1H), 8.30 (s, 1H), 7.76-7.72 (m, 2H), 7.64 (s, 1H), 7.45 (s, 1H), 7.37 (d, J = 9.3 Hz, 1H), 7.14 (s, 1H), 6.97-6.94 (m, 2H), 6.74 (d, J = 2.4 Hz, 1H), 6.34-6.32 (m, 1H), 4.36 (d, J = 5.7 Hz, 2H), 3.84 (s, 3H), 2.60- 2.52 (m, 1H), 2.53-2.47 (m, 1H), 1.97- 1.82 (m, 1H), 1.52- 1.49 (m, 2H), 0.93- 0.84 (m, 2H), 0.68-0.64 (m, 2H). |
| Example 225 | 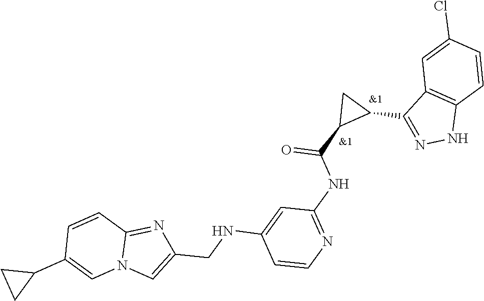 [SMILES: O=C(Nc1cc(NCc2cn3cc(C4CC4)ccc3n2)ccn1)[C@H]1C[C@@H]1c1n[nH]c2ccc(Cl)cc12] rac-(1S*,2S*)-2-(5-chloro-1H-indazol- 3-yl)-N-(4-(((6- cyclopropylimidazo[1,2-a]pyridin-2- yl)methyl)amino)pyridin-2- yl)cyclopropane-1-carboxamide (I- 225) | 2-bromo-N-((6- cyclopropylimidazo[1,2-a]pyridin-2- yl)methyl)pyridin-4-amine ESI-MS [M + H]+: 498.1, ¹H NMR (400 MHz, DMSO) δ 8.69 (d, J = 9.1 Hz, 1H), 8.32 (s, 1H), 7.99 (d, J = 5.8 Hz, 1H), 7.97 (d, J = 1.7 Hz, 1H), 7.72 (s, 1H), 7.71 (s, 1H), 7.50 (dd, J = 9.0 Hz, 1.9 Hz, 1H), 7.40 (d, J = 9.0 Hz, 2H), 7.14 (s, 1H), 7.03 (s, 1H), 6.98 (dd, J = 9.4 Hz, 1.5 Hz, 1H), 6.56-6.54 (m, 1H), 4.46 (d, J = 5.5 Hz, 2H), 2.71-2.67 (m, 1H), 2.25-2.21 (m, 1H), 1.93-1.88 (m, 1H), 1.52-1.45 (m, 2H), 0.92- 0.88 (m, 2H), 0.68-0.64 (m, 2H). |
| Example 226 | 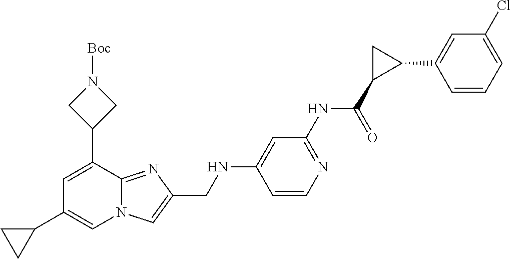 [SMILES: CC(C)(C)OC(=O)N1CC(c2cc(C3CC3)cn3cc(CNc4ccnc(NC(=O)[C@H]5C[C@@H]5c5cccc(Cl)c5)c4)nc23)C1] tert-butyl 3-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)azetidine-1-carboxylate (I-226) | 2-bromo-N-((6- cyclopropylimidazo[1,2-a]pyridin-2- yl)methyl)pyridin-4-amine ESI-MS [M + H]+: 613.3, ¹H NMR (400 MHz, DMSO) δ 10.31 (s, 1H), 8.21 (s, 1H), 7.76 (d, J = 5.7 Hz, 1H), 7.62 (s, 1H), 7.43 (s, 1H), 7.34-7.21 (m, 3H), 7.14-7.09 (m, 2H), 6.95 (s, 1H), 6.35- 6.30 (m, 1H), 4.35 (d, J = 5.6 Hz, 2H), 4.25-4.41 (m, 5H), 2.38-2.32 (m, 2H), 1.96-1.88 (m, 1H), 1.60-1.27 (m, 11H), 0.96-0.85 (m, 2H), 0.75-0.64 (m, 2H). |
| Example 227 | 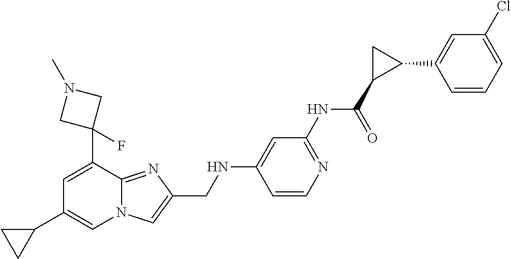 [SMILES: CN1CC(F)(c2cc(C3CC3)cn3cc(CNc4ccnc(NC(=O)[C@H]5C[C@@H]5c5cccc(Cl)c5)c4)nc23)C1] (1S,2S)-2-(3-chlorophenyl)-N-(4-(((6- cyclopropyl-8-(3-fluoro-1- methylazetidin-3-yl)imidazo[1,2- a]pyridin-2-yl)methyl)amino)pyridin- 2-yl)cyclopropane-1-carboxamide (I- 227) | 2-bromo-N-((6-cyclopropyl-8-(3- fluoro-1-methylazetidin-3- yl)imidazo[1,2-a]pyridin-2- yl)methyl)pyridin-4-amine ESI-MS [M + H]+: 545.2, ¹H NMR (400 MHz, DMSO) δ 10.34 (s, 1H), 8.37 (s, 1H), 7.77 (d, J = 5.8 Hz, 1H), 7.69 (s, 1H), 7.45 (s, 1H), 7.34-7.23 (m, 3H), 7.21-7.06 (m, 3H), 6.36-6.32 (m, 1H), 4.39 (d, J = 5.7 Hz, 2H), 4.20-4.12 (m, 2H), 3.89-3.80 (m, 2H), 2.49 (s, 3H), 2.41-2.31 (m, 2H), 1.98-1.92 (m, 1H), 1.47-1.40 (m, 1H), 1.36-1.32 (m, 1H), 0.97-0.87 (m, 2H), 0.74-0.66 (m, 2H). |
| Example 228 | 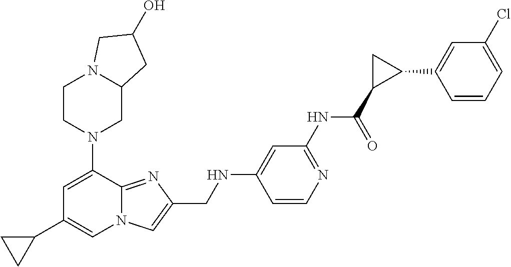 [SMILES: O=C(Nc1cc(NCc2cn3cc(C4CC4)cc(N4CCN5CC(O)CC5C4)c3n2)ccn1)[C@H]1C[C@@H]1c1cccc(Cl)c1] (1S,2S)-2-(3-chlorophenyl)-N-(4-(((6- cyclopropyl-8-(7- hydroxyhexahydropyrrolo[1,2- a]pyrazin-2(1H)-yl)imidazo[1,2- a]pyridin-2-yl)methyl)amino)pyridin- 2-yl)cyclopropane-1-carboxamide (I- 228) | 2-(2-(((2-bromopyridin-4- yl)amino)methyl)-6- cyclopropylimidazo[1,2-a]pyridin-8- yl)octahydropyrrolo[1,2-a]pyrazin-7-ol ESI-MS (M + H)+: 598.2, ¹H NMR (400 MHz, MeOD) δ 7.75 (d, J = 5.7 Hz, 2H), 7.54 (s, 1H), 7.37 (s, 1H), 7.25 (t, J = 7.7 Hz, 1H), 7.19-7.17 (m, 2H), 7.09 (d, J = 7.6 Hz, 1H), 6.40-6.38 (m, 2H), 4.48 (s, 2H), 4.42-4.39 (m, 1H), 4.10 (d, J = 10.9 Hz, 1H), 3.99 (d, J = 11.9 Hz, 1H), 3.11 (d, J = 11.0 Hz, 1H), 3.03 (d, J = 10.2 Hz, 1H), 2.95-2.89 (m, 1H), 2.64- 2.51 (m, 2H), 2.48-2.36 (m, 4H), 2.18-2.14 (m, 1H), 1.90-1.85 (m, 1H), 1.60-1.57 (m, 1H), 1.48-1.42 (m, 1H), 1.37-1.34 (m, 1H), 0.94- 0.89 (m, 2H), 0.72-0.66 (m, 2H). |
| Example 229 | 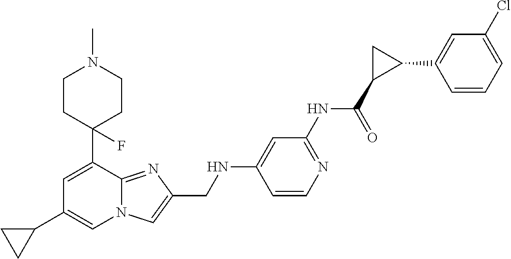 [SMILES: CN1CCC(F)(c2cc(C3CC3)cn3cc(CNc4ccnc(NC(=O)[C@H]5C[C@@H]5c5cccc(Cl)c5)c4)nc23)CC1] (1S,2S)-2-(3-chlorophenyl)-N-(4-(((6- cyclopropyl-8-(4-fluoro-1- methylpiperidin-4-yl)imidazo[1,2- a]pyridin-2-yl)methyl)amino)pyridin- 2-yl)cyclopropane-1-carboxamide (I- 229) | 2-bromo-N-((6-cyclopropyl-8-(4- fluoro-1-methylpiperidin-4- yl)imidazo[1,2-a]pyridin-2- yl)methyl)pyridin-4-amine ESI-MS (M + H)+: 573.3, ¹H NMR (400 MHz, DMSO) δ 10.32 (s, 1H), 8.27 (s, 1H), 7.75 (d, J = 5.8 Hz, 1H), 7.63 (s, 1H), 7.43 (s, 1H), 7.32-7.28 (m, 1H), 7.25-7.23 (m, 2H), 7.17-7.12 (m, 2H), 7.00 (s, 1H), 6.33-6.31 (m, 1H), 4.37 (d, J = 5.7 Hz, 2H), 3.17-3.09 (m, 1H), 3.06-2.98 (m, 1H), 2.81-2.78 (m, 2H), 2.38-2.32 (m, 4H), 2.30 (s, 3H), 1.97-1.90 (s, 1H), 1.80-1.74 (m, 2H), 1.45-1.41 (m, 1H), 1.36-1.32 (m, 1H), 0.92-0.88 (m, 2H), 0.67-0.63 (m, 2H). |
| Example 230 | 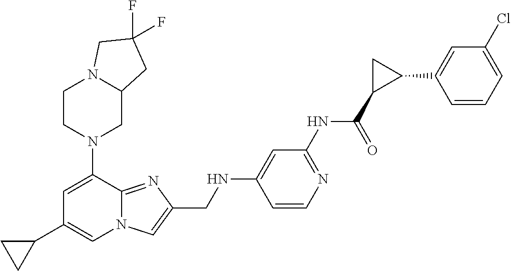 [SMILES: O=C(Nc1cc(NCc2cn3cc(C4CC4)cc(N4CCN5CC(F)(F)CC5C4)c3n2)ccn1)[C@H]1C[C@@H]1c1cccc(Cl)c1] (1S,2S)-2-(3-chlorophenyl)-N-(4-(((6- cyclopropyl-8-(7,7- difluorohexahydropyrrolo[1,2- a]pyrazin-2(1H)-yl)imidazo[1,2- a]pyridin-2-yl)methyl)amino)pyridin- 2-yl)cyclopropane-1-carboxamide (I- 230) | 2-bromo-N-((6-cyclopropyl-8-(7,7- difluorohexahydropyrrolo[1,2- a]pyrazin-2(1H)-yl)imidazo[1,2- a]pyridin-2-yl)methyl)pyridin-4-amine ESI-MS (M + H)+: 618.3, ¹H NMR (400 MHz, DMSO) δ 10.35 (s, 1H), 7.89 (s, 1H), 7.75 (d, J = 5.8 Hz, 1H), 7.54 (s, 1H), 7.41 (s, 1H), 7.31-7.28 (m, 1H), 7.24-7.22 (m, 2H), 7.19-7.10 (m, 2H), 6.34 (d, J = 5.2 Hz, 1H), 6.19 (s, 1H), 4.40 (d, J = 12.0 Hz, 2H), 4.34 (d, J = 5.7 Hz, 2H), 3.51-3.41 (m, 2H), 3.03 (d, J = 10.7 Hz, 1H), 2.80 (t, J = 10.7 Hz, 1H), 2.62-2.57 (m, 3H), 2.40- 2.33 (m, 3H), 2.09-1.92 (m, 1H), 1.87-1.82 (m, 1H), 1.47-1.42 (m, 1H), 1.37-1.35 (m, 1H), 0.89-0.84 (m, 2H), 0.68-0.64 (m, 2H). |
| Example 231 | 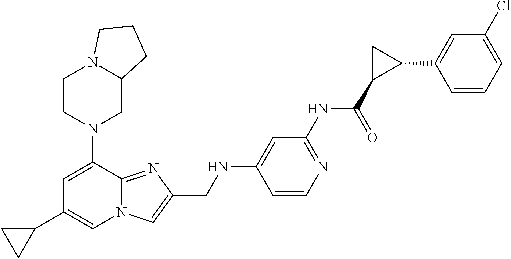 [SMILES: O=C(Nc1cc(NCc2cn3cc(C4CC4)cc(N4CCN5CCCC5C4)c3n2)ccn1)[C@H]1C[C@@H]1c1cccc(Cl)c1] (1S,2S)-2-(3-chlorophenyl)-N-(4-(((6- cyclopropyl-8-(hexahydropyrrolo[1,2- a]pyrazin-2(1H)-yl)imidazo[1,2- a]pyridin-2-yl)methyl)amino)pyridin- 2-yl)cyclopropane-1-carboxamide (I- 231) | 2-bromo-N-((6-cyclopropyl-8- (hexahydropyrrolo[1,2-a]pyrazin- 2(1H)-yl)imidazo[1,2-a]pyridin-2- yl)methyl)pyridin-4-amine ESI-MS [M + H]+: 582.3, ¹H NMR (400 MHz, DMSO) δ 10.29 (s, 1H), 7.84 (s, 1H), 7.73 (d, J = 5.8 Hz, 1H), 7.51 (s, 1H), 7.42 (s, 1H), 7.29 (t, J = 7.6 Hz, 1H), 7.24-7.22 (m, 2H), 7.13-7.06 (m, 2H), 6.32-6.30 (m, 1H), 6.16 (s, 1H), 4.32-4.31 (m, 2H), 3.05-3.00 (m, 2H), 2.74-2.65 (m, 2H), 2.34- 2.31 (m, 2H), 2.10-2.05 (m, 2H), 1.89- 1.80 (m, 2H), 1.71-1.69 (m, 2H), 1.45-1.32 (m, 4H), 1.21-1.21 (m, 2H), 0.86-0.82 (m, 2H), 0.65-0.61 (m, 2H). |
| Example 232 | 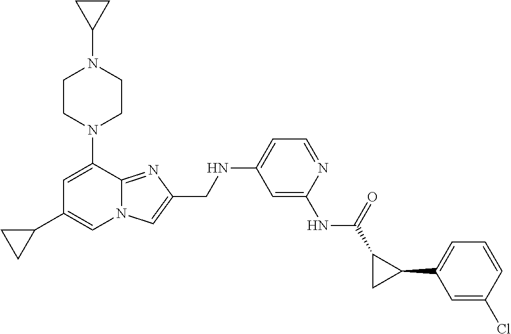 [SMILES: O=C(Nc1cc(NCc2cn3cc(C4CC4)cc(N4CCN(C5CC5)CC4)c3n2)ccn1)[C@H]1C[C@@H]1c1cccc(Cl)c1] (1S,2S)-2-(3-chlorophenyl)-N-(4-(((6- cyclopropyl-8-(4- cyclopropylpiperazin-1- yl)imidazo[1,2-a]pyridin-2- yl)methyl)amino)pyridin-2- yl)cyclopropane-1-carboxamide (I- 232) | 2-bromo-N-((6-cyclopropyl-8-(4- cyclopropylpiperazin-1-yl)imidazo[1,2- a]pyridin-2-yl)methyl)pyridin-4-amine ESI-MS [M + H]+: 582.3, ¹H NMR (400 MHz, DMSO) δ 10.36 (s, 1H), 7.86 (s, 1H), 7.75 (d, J = 5.8 Hz, 1H), 7.53 (s, 1H), 7.42 (s, 1H), 7.34-7.28 (m, 1H), 7.28-7.22 (m, 2H), 7.19-7.09 (m, 2H), 6.34 (d, J = 4.0 Hz, 1H), 6.14 (s, 1H), 4.34 (d, J = 5.6 Hz, 2H), 3.43 (s, 4H), 2.72 (s, 4H), 2.44-2.30 (m, 2H), 1.90-1.78 (m, 1H), 1.70 (s, 1H), 1.50- 1.41 (m, 1H), 1.40-1.30 (m, 1H), 0.91- 0.82 (m, 2H), 0.69-0.62 (m, 2H), 0.49-0.41 (m, 2H), 0.39-0.31 (m, 2H). |
| Example 233 | 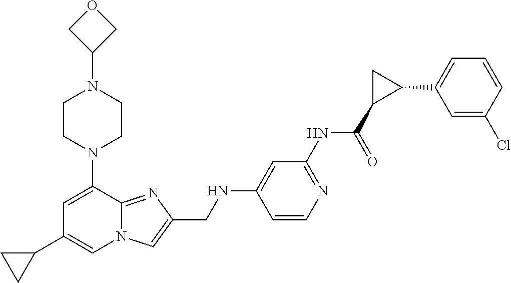 [SMILES: O=C(Nc1cc(NCc2cn3cc(C4CC4)cc(N4CCN(C5COC5)CC4)c3n2)ccn1)[C@H]1C[C@@H]1c1cccc(Cl)c1] (1S,2S)-2-(3-chlorophenyl)-N-(4-(((6- cyclopropyl-8-(4-(oxetan-3- yl)piperazin-1-yl)imidazo[1,2- a]pyridin-2-yl)methyl)amino)pyridin- 2-yl)cyclopropane-1-carboxamide (I- 233) | 2-bromo-N-((6-cyclopropyl-8-(4- (oxetan-3-yl)piperazin-1- yl)imidazo[1,2-a]pyridin-2- yl)methyl)pyridin-4-amine ESI-MS [M + H]+: 598.2, ¹H NMR (400 MHz, DMSO) δ 10.68 (s, 1H), 7.87 (d, J = 5.7 Hz, 1H), 7.76 (t, J = 5.8 Hz, 1H), 7.56 (d, J = 5.6 Hz, 2H), 7.43-6.92 (m, 5H), 6.43 (s, 1H), 6.17 (d, J = 5.7 Hz, 1H), 4.77-4.17 (m, 6H), 3.56-3.48 (m, 4H), 2.46-2.37 (m, 4H), 2.30-2.26 (m, 2H), 1.86-1.82 (m, 1H), 1.53-1.33 (m, 2H), 0.93-0.76 (m, 2H), 0.68-0.62 (m, 2H). |
| Example 234 | 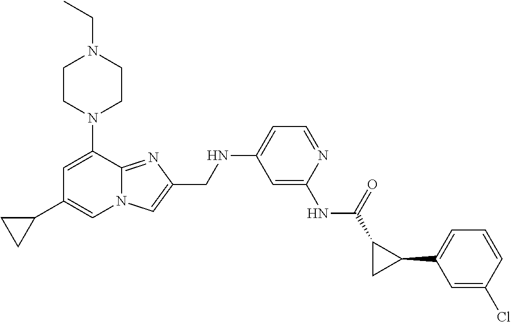 [SMILES: CCN1CCN(c2cc(C3CC3)cn3cc(CNc4ccnc(NC(=O)[C@H]5C[C@@H]5c5cccc(Cl)c5)c4)nc23)CC1] (1S,2S)-2-(3-chlorophenyl)-N-(4-(((6- cyclopropyl-8-(4-ethylpiperazin-1- yl)imidazo[1,2-a]pyridin-2- yl)methyl)amino)pyridin-2- yl)cyclopropane-1-carboxamide (I- 234) | 2-bromo-N-((6-cyclopropyl-8-(4- ethylpiperazin-1-yl)imidazo[1,2- a]pyridin-2-yl)methyl)pyridin-4-amine ESI-MS [M + H]+: 570.3, ¹H NMR (400 MHz, DMSO) δ 10.32 (s, 1H), 7.87 (s, 1H), 7.75 (d, J = 5.7 Hz, 1H), 7.53 (s, 1H), 7.45 (s, 1H), 7.38-7.20 (m, 3H), 7.20-7.04 (m, 2H), 6.32 (d, J = 4.1 Hz, 1H), 6.15 (s, 1H), 4.34 (d, J = 5.5 Hz, 2H), 3.48 (s, 4H), 2.55 (s, 4H), 2.46- 2.31 (m, 2H), 1.90-1.78 (m, 1H), 1.86- 1.82 (m, 2H), 1.53-1.39 (m, 1H), 1.39-1.31 (m, 1H), 1.05 (t, J = 7.1 Hz, 3H), 0.93-0.80 (m, 2H), 0.73-0.59 (m, 2H). |
| Example 235 | 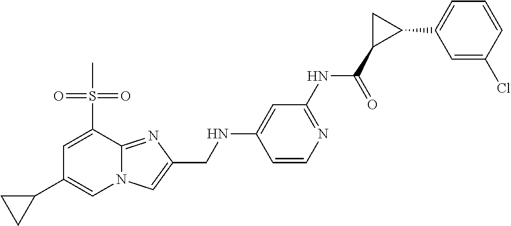 [SMILES: CS(=O)(=O)c1cc(C2CC2)cn2cc(CNc3ccnc(NC(=O)[C@H]4C[C@@H]4c4cccc(Cl)c4)c3)nc12] (1S,2S)-2-(3-chlorophenyl)-N-(4-(((6- cyclopropyl-8- (methylsulfonyl)imidazo[1,2-a]pyridin- 2-yl)methyl)amino)pyridin-2- yl)cyclopropane-1-carboxamide (I- 235) | 2-bromo-N-((6-cyclopropyl-8- (methylsulfonyl)imidazo[1,2-a]pyridin- 2-yl)methyl)pyridin-4-amine ESI-MS [M + H]+: 536.1, ¹H NMR (400 MHz, DMSO) δ 10.33 (s, 1H), 8.65 (d, J = 1.2 Hz, 1H), 7.83 (s, 1H), 7.77 (d, J = 5.8 Hz, 1H), 7.57 (d, J = 1.7 Hz, 1H), 7.45 (s, 1H), 7.33-7.29 (m, 1H), 7.26- 7.23 (m, 3H), 7.15-7.12 (m, 1H), 6.34 (dd, J = 5.8, 2.1 Hz, 1H), 4.45 (d, J = 5.6 Hz, 2H), 3.54 (s, 3H), 2.38-2.32 (m, 2H), 2.10-2.05 (m, 1H), 1.46-1.42 (m, 1H), 1.37-1.33 (m, 1H), 0.99- 0.95 (m, 2H), 0.73-0.69 (m, 2H). |
| Example 236 | 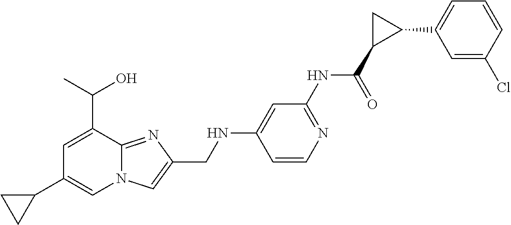 [SMILES: CC(O)c1cc(C2CC2)cn2cc(CNc3ccnc(NC(=O)[C@H]4C[C@@H]4c4cccc(Cl)c4)c3)nc12] (1S,2S)-2-(3-chlorophenyl)-N-(4-(((6- cyclopropyl-8-(1- hydroxyethyl)imidazo[1,2-a]pyridin-2- yl)methyl)amino)pyridin-2- yl)cyclopropane-1-carboxamide (I- 236) | 1-(2-(((2-bromopyridin-4- yl)amino)methyl)-6- cyclopropylimidazo[1,2-a]pyridin-8- yl)ethan-1-ol ESI-MS [M + H]+: 502.2, ¹H NMR (400 MHz, DMSO) δ 10.38 (s, 1H), 8.18 (s, 1H), 7.76 (d, J = 5.8 Hz, 1H), 7.62 (s, 1H), 7.39 (s, 1H), 7.33-7.29 (m, 1H), 7.25 (d, J = 6.8 Hz, 2H), 7.14 (d, J = 7.5 Hz, 1H), 7.01 (s, 1H), 6.34 (d, J = 4.4 Hz, 1H), 5.30 (d, J = 4.7 Hz, 1H), 5.20- 5.17 (m, 1H), 4.37 (d, J = 5.7 Hz, 2H), 2.40-2.34 (m, 2H), 1.95-1.88 (m, 1H), 1.47-1.45 (m, 4H), 1.38-1.33 (m, 6.2 Hz, 1H), 0.93-0.89 (m, 2H), 0.66-0.62 (m, 2H). |
| Example 237 | 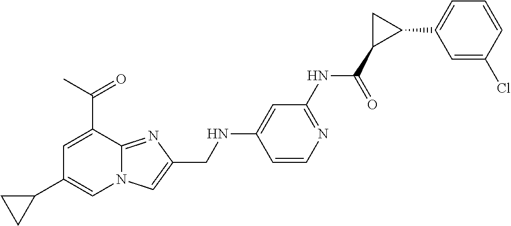 [SMILES: CC(=O)c1cc(C2CC2)cn2cc(CNc3ccnc(NC(=O)[C@H]4C[C@@H]4c4cccc(Cl)c4)c3)nc12] (1S,2S)-N-(4-(((8-acetyl-6- cyclopropylimidazo[1,2-a]pyridin-2- yl)methyl)amino)pyridin-2-yl)-2-(3- chlorophenyl)cyclopropane-1- carboxamide (I-237) | 1-(2-(((2-bromopyridin-4- yl)amino)methyl)-6- cyclopropylimidazo[1,2-a]pyridin-8- yl)ethan-1-one ESI-MS [M + H]+: 500.2, ¹H NMR (400 MHz, CDCl₃) δ 8.73 (s, 1H), 8.04 (s, 1H), 7.80 (d, J = 5.8 Hz, 1H), 7.61 (s, 2H), 7.53 (s, 1H), 7.22-7.17 (m, 2H), 7.08 (s, 1H), 7.01 (d, J = 6.9 Hz, 1H), 6.30-6.29 (m, 1H), 5.18-5.15 (m, 1H), 4.56 (d, J = 5.3 Hz, 2H), 3.02 (s, 3H), 2.59-2.54 (m, 1H), 1.93-1.90 (m, 1H), 1.82-1.79 (m, 2H), 1.37- 1.33 (m, 1H), 1.03-0.98 (m, 2H), 0.74- 0.71 (m, 2H). |
| Example 238 | 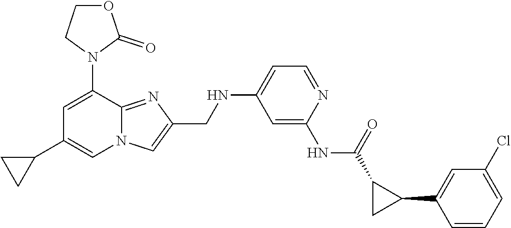 [SMILES: O=C(Nc1cc(NCc2cn3cc(C4CC4)cc(N4CCOC4=O)c3n2)ccn1)[C@H]1C[C@@H]1c1cccc(Cl)c1] (1S,2S)-2-(3-chlorophenyl)-N-(4-(((6- cyclopropyl-8-(2-oxooxazolidin-3- yl)imidazo[1,2-a]pyridin-2- yl)methyl)amino)pyridin-2- yl)cyclopropane-1-carboxamide (I- 238) | 3-(2-(((2-bromopyridin-4- yl)amino)methyl)-6- cyclopropylimidazo[1,2-a]pyridin-8- yl)oxazolidin-2-one ESI-MS [M + H]+: 543.2, ¹H NMR (400 MHz, DMSO) δ 10.32 (s, 1H), 8.26 (s, 1H), 8.15 (s, 1H), 7.76 (d, J = 5.8 Hz, 1H), 7.69 (s, 1H), 7.45 (s, 1H), 7.29- 7.13 (m, 6H), 6.32 (d, J = 4.0 Hz, 1H), 4.59-4.46 (m, 4H), 4.38-4.36 (m, 2H), 2.40-2.29 (m, 2H), 1.99-1.84 (m, 1H), 1.49-1.38 (m, 1H), 1.38-1.24 (m, 1H), 0.99-0.83 (m, 2H), 0.68- 0.58 (m, 2H). |
| Example 239 | 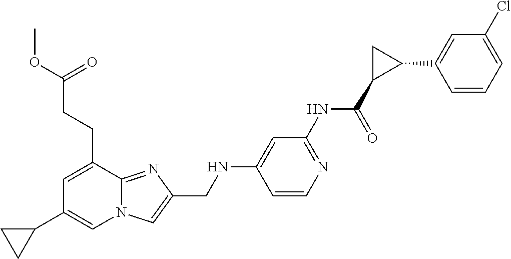 [SMILES: COC(=O)CCc1cc(C2CC2)cn2cc(CNc3ccnc(NC(=O)[C@H]4C[C@@H]4c4cccc(Cl)c4)c3)nc12] methyl 3-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)propanoate (I-239) | methyl 3-(2-(((2-bromopyridin-4- yl)amino)methyl)-6- cyclopropylimidazo[1,2-a]pyridin-8- yl)propanoate ESI-MS [M + H]+: 544.3, ¹H NMR (400 MHz, DMSO) δ 10.31 (s, 1H), 8.18 (s, 1H), 7.75 (d, J = 5.7 Hz, 1H), 7.61-7.58 (m, 1H), 7.44 (s, 1H), 7.37-7.18 (m, 3H), 7.15-7.10 (m, 2H), 6.80 (s, 1H), 6.32 (d, J = 5.8 Hz, 1H), 4.36 (d, J = 5.5 Hz, 2H), 3.60 (s, 3H), 3.09 (t, J = 7.6 Hz, 2H), 2.82 (t, J = 7.6 Hz, 2H), 2.36-2.35 (m, 2H), 1.87-1.85 (m, 1H), 1.52-1.37 (m, 1H), 1.34-1.33 (m, 1H), 0.90-0.88 (m, 2H), 0.66-0.63 (m, 2H). |
| Example 240 | 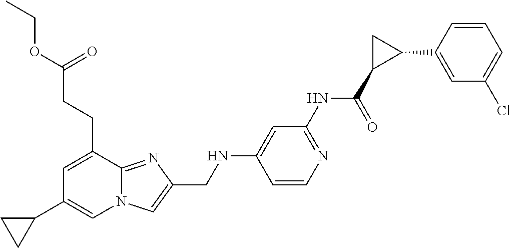 [SMILES: CCOC(=O)CCc1cc(C2CC2)cn2cc(CNc3ccnc(NC(=O)[C@H]4C[C@@H]4c4cccc(Cl)c4)c3)nc12] ethyl 3-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)propanoate (I-240) | ethyl 3-(2-(((2-bromopyridin-4- yl)amino)methyl)-6- cyclopropylimidazo[1,2-a]pyridin-8- yl)propanoate ESI-MS [M + H]+: 558.2. ¹H NMR (400 MHz, DMSO) δ 10.30 (s, 1H), 8.38 (s, 1H), 8.18 (d, J = 1.3 Hz, 1H), 7.75 (d, J = 5.8 Hz, 1H), 7.61 (s, 1H), 7.43 (s, 1H), 7.34-7.28 (m, 1H), 7.26-7.22 (m, 2H), 7.15-7.12 (m, 2H), 6.80 (s, 1H), 6.33-6.31 (m, 1H), 4.36 (d, J = 5.7 Hz, 2H), 4.09-4.03 (m, 2H), 3.12-3.06 (m, 2H), 2.82-1.78 (m, 2H), 2.38-2.32 (m, 2H), 1.90-1.83 (m, 1H), 1.48-1.41 (m, 1H), 1.36-1.32 (m, 1H), 1.18-1.15 (m, 3H),0.95-0.85 (m, 2H), 0.64 (m, 2H). |
| Example 241 | 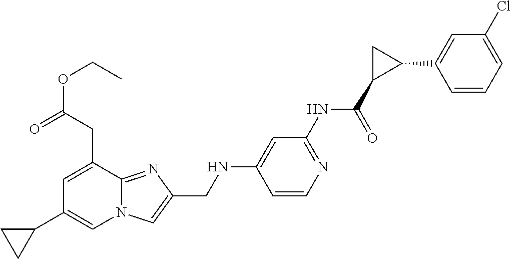 [SMILES: CCOC(=O)Cc1cc(C2CC2)cn2cc(CNc3ccnc(NC(=O)[C@H]4C[C@@H]4c4cccc(Cl)c4)c3)nc12] ethyl 2-(2-(((2-((1S,2S)-2-(3- chlorophenyl)cyclopropane-1- carboxamido)pyridin-4- yl)amino)methyl)-6- cyclopropylimidazo[1,2-a]pyridin-8- yl)acetate (I-241) | ethyl 2-(2-(((2-bromopyridin-4- yl)amino)methyl)-6- cyclopropylimidazo[1,2-a]pyridin-8- yl)acetate ESI-MS [M + H]+: 544.2. ¹H NMR (400 MHz, DMSO) δ 8.25 (s, 1H), 7.79 (d, J = 6.3 Hz, 1H), 7.69 (s, 1H), 7.34-7.26 (m, 4H), 7.18-7.16 (m, 1H), 6.93 (s, 1H), 6.49 (s, 1H), 4.43-4.42 (m, 2H), 4.11-4.06 (m, 2H), 3.90 (s, 2H), 2.51- 2.49 (m, 1H), 2.30-2.27 (m, 1H), 1.94- 1.88 (m, 1H), 1.52-1.45 (m, 2H), 1.18 (t, J = 7.1 Hz, 3H), 0.94-0.90 (m, 2H), 0.67-0.63 (m, 2H). |
| Example 242 | 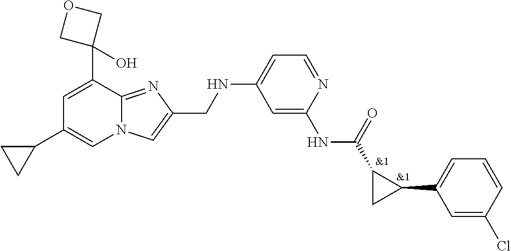 [SMILES: O=C(Nc1cc(NCc2cn3cc(C4CC4)cc(C4(O)COC4)c3n2)ccn1)[C@H]1C[C@@H]1c1cccc(Cl)c1] rac-(1S*,2S*)-2-(3-chlorophenyl)-N- (4-(((6-cyclopropyl-8-(3- hydroxyoxetan-3-yl)imidazo[1,2- a]pyridin-2-yl)methyl)amino)pyridin- 2-yl)cyclopropane-1-carboxamide (I- 242) | 3-(2-(((2-aminopyridin-4- yl)amino)methyl)-6- cyclopropylimidazo[1,2-a]pyridin-8- yl)oxetan-3-ol ESI-MS [M + H]+: 530.2., ¹H NMR (400 MHz, DMSO) δ 10.31 (s, 1H), 8.28 (s, 1H), 7.76 (d, J = 5.8 Hz, 1H), 7.65 (s, 1H), 7.45 (s, 1H), 7.33-7.22 (m, 3H), 7.15-7.11 (m, 2H), 7.05 (s, 1H), 6.46 (s, 1H), 6.33 (d, J = 5.7 Hz, 1H), 5.25 (d, J = 6.4 Hz, 2H), 4.67 (d, J = 6.4 Hz, 2H), 4.38 (d, J = 5.8 Hz, 2H), 2.39-2.32 (m, 2H), 1.97-1.89 (m, 1H), 1.47-1.41 (m, 1H), 1.35-1.31 (m, 1H), 0.95-0.87 (m, 2H), 0.72-0.66 (m, 2H). |
| Example 243 | 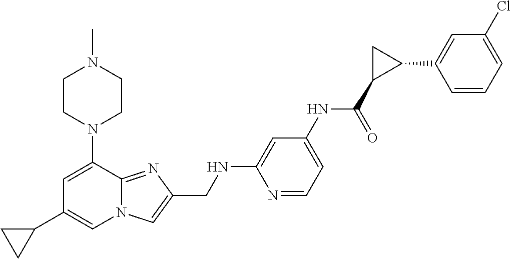 [SMILES: CN1CCN(c2cc(C3CC3)cn3cc(CNc4cc(NC(=O)[C@H]5C[C@@H]5c5cccc(Cl)c5)ccn4)nc23)CC1] (1S,2S)-2-(3-chlorophenyl)-N-(2-(((6- cyclopropyl-8-(4-methylpiperazin-1- yl)imidazo[1,2-a]pyridin-2- yl)methyl)amino)pyridin-4- yl)cyclopropane-1-carboxamide (I- 243) | 4-bromo-N-((6-cyclopropyl-8-(4- methylpiperazin-1-yl)imidazo[1,2- a]pyridin-2-yl)methyl)pyridin-2-amine ESI-MS [M + H]+: 556.3. ¹H NMR (400 MHz, DMSO) δ 10.26 (s, 1H), 7.97- 7.73 (m, 2H), 7.47 (s, 1H), 7.41-7.23 (m, 3H), 7.17 (d, J = 7.6 Hz, 1H), 6.96- 6.81 (m, 2H), 6.67 (d, J = 5.7 Hz, 1H), 6.13 (s, 1H), 4.46 (d, J = 5.6 Hz, 2H), 3.47 (s, 4H), 2.67 (s, 4H), 2.39-2.36 (m, 1H), 2.23 (s, 3H), 2.16-2.09 (m, 1H), 1.88-1.77 (m, 1H), 1.51-1.42 (m, 2H), 0.94-0.76 (m, 2H), 0.73-0.49 (m, 2H). |
| Example 244 | 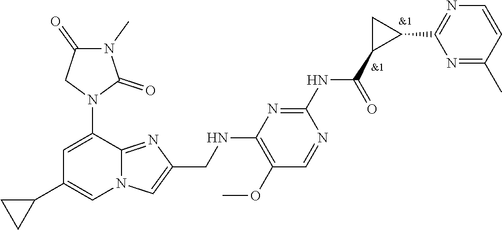 [SMILES: COc1cnc(NC(=O)[C@H]2C[C@@H]2c2nccc(C)n2)nc1NCc1cn2cc(C3CC3)cc(N3CC(=O)N(C)C3=O)c2n1] rac-(1S*,2S*)-N-(4-(((6-cyclopropyl- 8-(3-methyl-2,4-dioxoimidazolidin-1- yl)imidazo[1,2-a]pyridin-2- yl)methyl)amino)-5- methoxypyrimidin-2-yl)-2-(4- methylpyrimidin-2-yl)cyclopropane-1- carboxamide (I-244) | 1-(2-(((2-chloro-5-methoxypyrimidin- 4-yl)amino)methyl)-6- cyclopropylimidazo[1,2-a]pyridin-8- yl)-3-methylimidazolidine-2,4-dione ESI-MS [M + H]+: 583.2, ¹H NMR (400 MHz, DMSO) δ 10.16 (s, 1H), 8.46 (d, J = 5.0 Hz, 1H), 8.31 (s, 1H), 8.20 (s, 1H), 7.70 (d, J = 21.9 Hz, 2H), 7.50 (s, 1H), 7.31 (s, 1H), 7.14 (d, J = 5.0 Hz, 1H), 4.88 (s, 2H), 4.58 (d, J = 5.1 Hz, 2H), 3.81 (s, 3H), 2.97 (s, 3H), 2.67- 2.59 (m,, 2H), 2.38 (s, 3H), 1.93-1.90 (m, 1H), 1.50-1.46 (m, 2H), 1.03- 0.80 (m, 2H), 0.65-0.61 (m, 2H). |
| Example 245 | 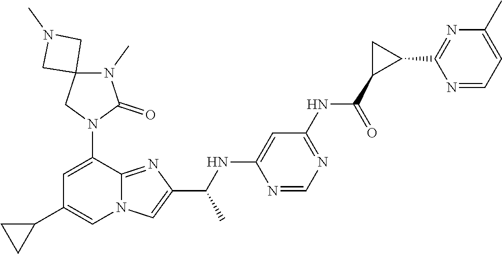 [SMILES: Cc1ccnc([C@H]2C[C@@H]2C(=O)Nc2cc(N[C@H](C)c3cn4cc(C5CC5)cc(N5CC6(CN(C)C6)N(C)C5=O)c4n3)ncn2)n1] (1S,2S)-N-(6-(((R)-1-(6-cyclopropyl-8- (2,5-dimethyl-6-oxo-2,5,7- triazaspiro[3.4]octan-7-yl)imidazo[1,2- a]pyridin-2-yl)ethyl)amino)pyrimidin- 4-yl)-2-(4-methylpyrimidin-2- yl)cyclopropane-1-carboxamide (I- 245) | R)-7-(2-(1-((6-bromopyrimidin-4- yl)amino)ethyl)-6- cyclopropylimidazo[1,2-a]pyridin-8- yl)-2,5-dimethyl-2,5,7- triazaspiro[3.4]octan-6-one ESI-MS [M + H]+: 608.4;. ¹H NMR (400 MHz, DMSO) δ 10.65 (s, 1H), 8.52 (d, J = 5.1 Hz, 1H), 8.15 (s, 1H), 7.80-7.58 (m, 2H), 7.47 (s, 1H), 7.29 (s, 1H), 7.21 (d, J = 5.1 Hz, 1H), 6.98 (s, 1H), 5.28 (s, 1H), 4.41-4.31 (m, 2H), 4.18-4.06 (m, 2H), 3.58 (s, 2H), 2.81 (s, 3H), 2.67 (s, 3H), 2.64-2.58 (m, 1H), 2.56-2.53 (m, 1H), 2.41 (s, 3H), 1.86-1.76 (m, 1H), 1.55-1.44 (m, 5H), 0.87-0.77 (m, 2H), 0.64- 0.55 (m, 2H). |
| Example 246 | 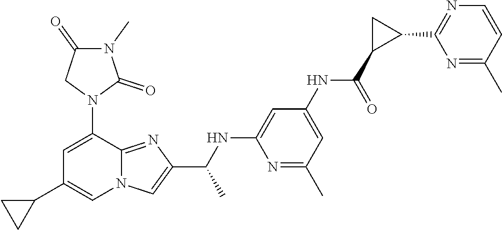 [SMILES: Cc1cc(NC(=O)[C@H]2C[C@@H]2c2nccc(C)n2)cc(N[C@H](C)c2cn3cc(C4CC4)cc(N4CC(=O)N(C)C4=O)c3n2)n1] (1S,2S)-N-(2-(((R)-1-(6-cyclopropyl-8- (3-methyl-2,4-dioxoimidazolidin-1- yl)imidazo[1,2-a]pyridin-2- yl)ethyl)amino)-6-methylpyridin-4-yl)- 2-(4-methylpyrimidin-2- yl)cyclopropane-1-carboxamide (I- 246) | (R)-1-(2-(1-((4-bromo-6- methylpyridin-2-yl)amino)ethyl)-6- cyclopropylimidazo[1,2-a]pyridin-8- yl)-3-methylimidazolidine-2,4-dione ESI-MS [M + H]+: 580.2, ¹H NMR (400 MHz, DMSO) δ 10.24 (s, 1H), 8.52 (d, J = 5.1 Hz, 1H), 8.30 (s, 1H), 8.23 (d, J = 1.1 Hz, 1H), 7.61 (s, 1H), 7.31 (d, J = 1.5 Hz, 1H), 7.21 (d, J = 5.1 Hz, 1H), 6.69 (d, J = 8.7 Hz, 2H), 6.52 (s, 1H), 5.19-5.04 (m, 1H), 4.94 (d, J = 2.0 Hz, 2H), 2.97 (s, 3H), 2.55- 2.51 (m, 2H), 2.41 (s, 3H), 2.17 (s, 3H), 1.99-1.86 (m, 1H), 1.54-1.41 (m, 5H), 0.98-0.88 (m, 2H), 0.68-0.57 (m, 2H). |
| Example 247 | 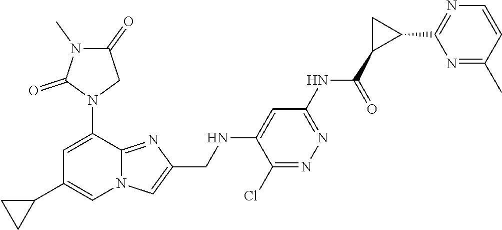 [SMILES: Cc1ccnc([C@H]2C[C@@H]2C(=O)Nc2cc(NCc3cn4cc(C5CC5)cc(N5CC(=O)N(C)C5=O)c4n3)c(Cl)nn2)n1] (1S,2S)-N-(6-chloro-5-(((6- cyclopropyl-8-(3-methyl-2,4- dioxoimidazolidin-1-yl)imidazo[1,2- a]pyridin-2- yl)methyl)amino)pyridazin-3-yl)-2-(4- methylpyrimidin-2-yl)cyclopropane-1- carboxamide (I-247) | 1-(6-cyclopropyl-2-(((3,6- dichloropyridazin-4- yl)amino)methyl)imidazo[1,2- a]pyridin-8-yl)-3-methylimidazolidine- 2,4-dione ESI-MS [M + H]+: 588.2, ¹H NMR (400 MHz, DMSO) δ 10.58 (s, 1H), 8.54 (d, J = 5.1 Hz, 1H), 8.29 (s, 1H), 7.74 (s, 1H), 7.37 (s, 1H), 7.32 (s, 1H), 7.22 (d, J = 5.1 Hz, 1H), 6.95 (s, 1H), 4.90 (d, J = 2.7 Hz, 2H), 4.50 (d, J = 5.7 Hz, 2H), 2.97 (s, 3H), 2.65-2.55 (m, 2H), 2.42 (s, 3H), 1.98-1.88 (m, 1H), 1.57-1.50 (m, 2H), 0.96-0.90 (m, 2H), 0.69-0.62 (m, 2H). |
| Example 248 | 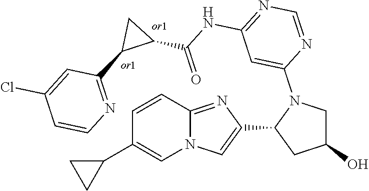 [SMILES: O=C(Nc1cc(N2C[C@@H](O)C[C@@H]2c2cn3cc(C4CC4)ccc3n2)ncn1)[C@H]1C[C@@H]1c1cc(Cl)ccn1] (1S*,2S*)-2-(4-chloropyridin-2-yl)-N- (6-((2R,4S)-2-(6- cyclopropylimidazo[1,2-a]pyridin-2- yl)-4-hydroxypyrrolidin-1- yl)pyrimidin-4-yl)cyclopropane-1- carboxamide, isomer 1 (I-248) | 2-((2R,4S)-1-(6-bromopyrimidin-4-yl)- 4-((tert- butyldimethylsilyl)oxy)pyrrolidin-2- yl)-6-cyclopropylimidazo[1,2- a]pyridine ESI-MS [M + H]+: 516.2, ¹H NMR (400 MHz, cd3od) δ 8.32 (d, J = 5.0 Hz, 1H), 8.25-8.04 (m, 2H), 7.63 (s, 1H), 7.35 (d, J = 9.3Hz, 2H), 7.31-7.23 (m, 2H), 7.05 (d, J = 9.3Hz, 1H), 5.30-5.01 (m, 1H), 4.90 (s, 1H), 4.60 (s, 1H), 4.12-3.88 (m, 1H), 2.55-2.30 (m, 4H), 1.99-1.87 (m, 1H), 1.62-1.57 (m, 2H), 0.97-0.92 (m, 2H), 0.76-0.64 (m, 2H). |
| Example 249 | 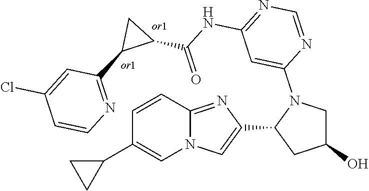 [SMILES: O=C(Nc1cc(N2C[C@@H](O)C[C@@H]2c2cn3cc(C4CC4)ccc3n2)ncn1)[C@H]1C[C@@H]1c1cc(Cl)ccn1] (1S*,2S*)-2-(4-chloropyridin-2-yl)-N- (6-((2R,4S)-2-(6- cyclopropylimidazo[1,2-a]pyridin-2- yl)-4-hydroxypyrrolidin-1- yl)pyrimidin-4-yl)cyclopropane-1- carboxamide, isomer 2 (I-249) | 2-((2R,4S)-1-(6-bromopyrimidin-4-yl)- 4-((tert- butyldimethylsilyl)oxy)pyrrolidin-2- yl)-6-cyclopropylimidazo[1,2- a]pyridine ESI-MS [M + H]+: 516.2, ¹H NMR (400 MHz, MeOD) δ 8.33 (d, J = 5.1 Hz, 1H), 8.20-8.07 (m, 2H), 7.62 (s, 1H), 7.43 (s, 1H), 7.34 (d, J = 9.3 Hz, 1H), 7.25 (d, J = 5.2 Hz, 2H), 7.05 (d, J = 9.5 Hz, 1H), 5.24-5.04 (m, 1H), 4.90 (s, 1H), 4.59 (s, 1H), 4.07-3.86 (m, 1H), 2.58 (s, 1H), 2.48-2.34 (m, 3H), 1.95- 1.89 (m, 1H), 1.56 (s, 2H), 0.99-0.93 (m, 2H), 0.73-0.65 (m, 2H). |
| Example 250 | 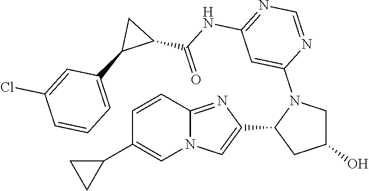 [SMILES: O=C(Nc1cc(N2C[C@H](O)C[C@@H]2c2cn3cc(C4CC4)ccc3n2)ncn1)[C@H]1C[C@@H]1c1cccc(Cl)c1] (1S,2S)-2-(3-chlorophenyl)-N-(6- ((2R,4R)-2-(6-cyclopropylimidazo[1,2- a]pyridin-2-yl)-4-hydroxypyrrolidin-1- yl)pyrimidin-4-yl)cyclopropane-1- carboxamide (I-250) | (3R,5R)-1-(6-bromopyrimidin-4-yl)-5- (6-cyclopropylimidazo[1,2-a]pyridin-2- yl)pyrrolidin-3-ol ESI-MS [M + H]+: 515.2. ¹H NMR (400 MHz, MeOD) δ 8.24-8.14 (m, 2H), 7.66-7.58 (m, 1H), 7.35 (d, J = 9.4 Hz, 1H), 7.30-6.89 (m, 6H), 5.18-5.52 (m, 1H), 4.89 (s, 1H), 4.59 (d, J = 4.1 Hz, 1H), 3.98-3.94 (m, 2H), 2.46-2.32 (m, 3H), 2.18-2.14 (m, 1H), 1.96-1.88 (m, 1H), 1.57 (s, 1H), 1.36 (s, 1H), 1.03- 0.86 (m, 2H), 0.79-0.58 (m, 2H). |
| Example 251 | 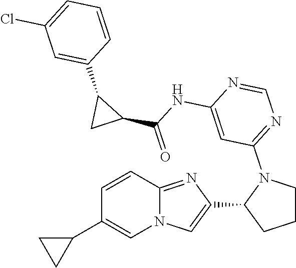 [SMILES: O=C(Nc1cc(N2CCC[C@@H]2c2cn3cc(C4CC4)ccc3n2)ncn1)[C@H]1C[C@@H]1c1cccc(Cl)c1] (1S,2S)-2-(3-chlorophenyl)-N-(6-((R)- 2-(6-cyclopropylimidazo[1,2- a]pyridin-2-yl)pyrrolidin-1- yl)pyrimidin-4-yl)cyclopropane-1- carboxamide (I-251) | (R)-2-(1-(6-chloropyrimidin-4- yl)pyrrolidin-2-yl)-6- cyclopropylimidazo[1,2-a]pyridine ESI-MS [M + H]+: 499.2, ¹H NMR (400 MHz, DMSO) δ 10.65 (d, J = 46.2 Hz, 1H), 8.34-8.09 (m, 2H), 7.57- 6.83 (m, 8H), 5.55-5.29 (m, 1H), 3.90- 3.69 (m, 1H), 3.68-3.49 (m, 1H), 2.46-2.24 (m, 2H), 2.21-2.09 (m, 1H), 2.06-1.85 (m, 3H), 1.58-1.35 (m, 3H), 0.99-0.80 (m, 2H), 0.74- 0.52 (m, 2H). |
| Example 252 | 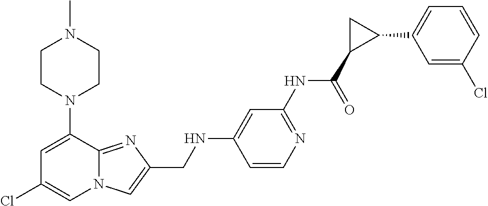 [SMILES: CN1CCN(c2cc(Cl)cn3cc(CNc4ccnc(NC(=O)[C@H]5C[C@@H]5c5cccc(Cl)c5)c4)nc23)CC1] (1S,2S)-N-(4-(((6-chloro-8-(4- methylpiperazin-1-yl)imidazo[1,2- a]pyridin-2-yl)methyl)amino)pyridin- 2-yl)-2-(3-chlorophenyl)cyclopropane- 1-carboxamide (I-252) | 2-bromo-N-((6-chloro-8-(4- methylpiperazin-1-yl)imidazo[1,2- a]pyridin-2-yl)methyl)pyridin-4-amine ESI-MS [M + H]+: 550.2, ¹H NMR (400 MHz, DMSO) δ 10.32 (s, 1H), 8.31 (d, J = 1.6 Hz, 1H), 7.76 (d, J = 5.8 Hz, 1H), 7.65 (s, 1H), 7.45 (s, 1H), 7.31 (t, J = 7.9 Hz, 1H), 7.25 (d, J = 7.5 Hz, 2H), 7.14 (d, J = 7.4 Hz, 2H), 6.40 (s, 1H), 6.34-6.31 (m, 1H), 4.37 (d, J = 5.3 Hz, 2H), 3.57 (s, 4H), 3.30 (s, 4H), 2.39-2.33 (m, 2H), 2.26 (s, 3H), 1.48- 1.41 (m, 1H), 1.37-1.32 (m, 1H). |
| Example 253 | 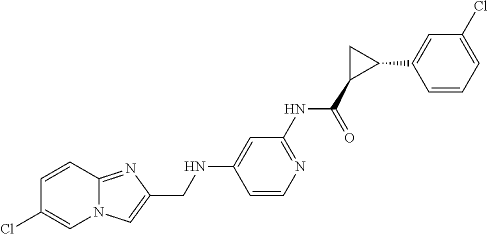 [SMILES: O=C(Nc1cc(NCc2cn3cc(Cl)ccc3n2)ccn1)[C@H]1C[C@@H]1c1cccc(Cl)c1] (1S,2S)-N-(4-(((6-chloroimidazo[1,2- a]pyridin-2-yl)methyl)amino)pyridin- 2-yl)-2-(3-chlorophenyl)cyclopropane- 1-carboxamide (I-253) | 2-bromo-N-((6-chloroimidazo[1,2- a]pyridin-2-yl)methyl)pyridin-4-amine ESI-MS [M + H]+: 452.1, ¹H NMR (400 MHz, DMSO) δ 10.33 (s, 1H), 8.80 (dd, J = 2.0, 0.7 Hz, 1H), 7.77- 7.75 (m, 2H), 7.54 (d, J = 9.6 Hz, 1H), 7.43 (s, 1H), 7.33-7.24 (m, 4H), 7.17- 7.13 (m, 2H), 6.33 (dd, J = 5.8, 2.0 Hz, 1H), 4.39 (d, J = 5.6 Hz, 2H), 2.38- 2.33 (m, 2H), 1.46-1.42 (m, 1H), 1.37-1.34 (m, 1H). |
| Example 254 | 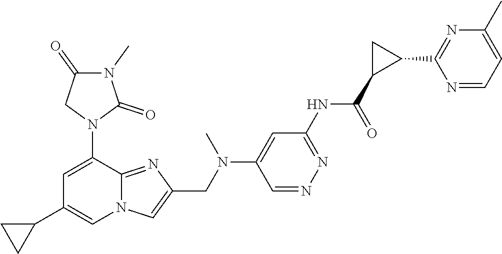 [SMILES: Cc1ccnc([C@H]2C[C@@H]2C(=O)Nc2cc(N(C)Cc3cn4cc(C5CC5)cc(N5CC(=O)N(C)C5=O)c4n3)cnn2)n1] (1S,2S)-N-(5-(((6-cyclopropyl-8-(3- methyl-2,4-dioxoimidazolidin-1- yl)imidazo[1,2-a]pyridin-2- yl)methyl)(methyl)amino)pyridazin-3- yl)-2-(4-methylpyrimidin-2- yl)cyclopropane-1-carboxamide (I- 254) | 1-(2-(((6-chloropyridazin-4- yl)(methyl)amino)methyl)-6- cyclopropylimidazo[1,2-a]pyridin-8- yl)-3-methylimidazolidine-2,4-dione ESI-MS [M + H]+: 567.3, ¹H NMR (400 MHz, DMSO) δ 10.97 (s, 1H), 8.67 (d, J = 2.8 Hz, 1H), 8.52 (d, J = 5.1 Hz, 1H), 8.35 (s, 1H), 8.22 (d, J = 1.1 Hz, 1H), 7.76 (s, 1H), 7.65 (d, J = 2.4 Hz, 1H), 7.38 (d, J = 1.5 Hz, 1H), 7.21 (d, J = 5.1 Hz, 1H), 4.88 (s, 2H), 4.72 (s, 2H), 3.13 (s, 3H), 2.96 (s, 3H), 2.71-2.64 (m, 1H), 2.41 (s, 3H), 2.37-2.30 (m, 1H), 1.98-1.90 (m, 1H), 1.56-1.48 (m, 2H), 0.98-0.89 (m, 2H), 0.68-0.60 (m, 2H). |
| Example 255 | 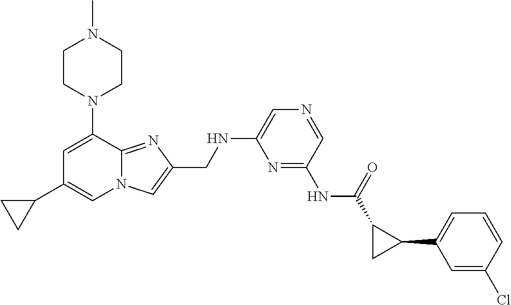 [SMILES: CN1CCN(c2cc(C3CC3)cn3cc(CNc4cncc(NC(=O)[C@H]5C[C@@H]5c5cccc(Cl)c5)n4)nc23)CC1] (1S,2S)-2-(3-chlorophenyl)-N-(6-(((6- cyclopropyl-8-(4-methylpiperazin-1- yl)imidazo[1,2-a]pyridin-2- yl)methyl)amino)pyrazin-2- yl)cyclopropane-1-carboxamide (I- 255) | 6-chloro-N-((6-cyclopropyl-8-(4- methylpiperazin-1-yl)imidazo[1,2- a]pyridin-2-yl)methyl)pyrazin-2-amine ESI-MS [M + H]+: 557.2. ¹H NMR (400 MHz, MeOD) δ 8.48 (s, 1H), 7.76 (s, 1H), 7.67-7.61 (m, 1H), 7.56 (s, 1H), 7.27-7.22 (m, 1H), 7.19 (d, J = 7.5 Hz, 2H), 7.10 (d, J = 7.6 Hz, 1H), 6.37 (s, 1H), 4.64 (s, 2H), 3.35 (d, J = 17.0 Hz, 4H), 2.48-2.46 (m, 2H), 2.39 (s, 3H), 2.20-2.19 (m, 2H), 2.02-2.01 (m, 1H), 1.93-1.82 (m, 2H), 1.65-1.56 (m, 2H), 0.93-0.89 (m, 2H), 0.67 (dt, J = 6.4, 4.7 Hz, 2H). |

### Example 256-258

### Synthesis of rac-(1S*,2S*)-2-(3-amino-5-chlorophenyl)-N-(6-(((6-cyclopropylimidazo[1,2-a]pyridin-2-yl)methylamino)pyrimidin-4-yl)cyclopropane-1-carboxamide (I-256)

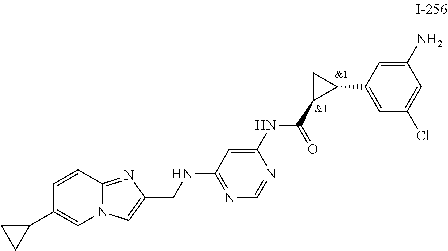 [SMILES: Nc1cc(Cl)cc([C@H]2C[C@@H]2C(=O)Nc2cc(NCc3cn4cc(C5CC5)ccc4n3)ncn2)c1]

The title compound was prepared in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl) amino)pyrimidin-4-yl)cyclopropane-1-carboxamide and obtained as a mixture of enantiomers. ESI-MS (M+H)+: 474.2, ¹H NMR (400 MHz, DMSO) δ 10.60 (s, 1H), 8.29 (s, 1H), 8.19 (s, 1H), 7.85 (s, 1H), 7.61 (s, 1H), 7.37 (d, J=9.3 Hz, 1H), 7.26 (s, 1H), 6.97-6.95 (m, 1H), 6.42-6.41 (m, 1H), 6.29 (d, J=9.7 Hz, 2H), 5.37 (s, 2H), 4.56 (s, 2H), 2.31-2.25 (m, 1H), 2.23-2.16 (m, 1H), 1.95-1.87 (m, 1H), 1.41-1.37 (m, 1H), 1.28-1.22 (m, 1H), 0.95-0.87 (m, 2H), 0.69-0.63 (m, 2H).

The mixture was separated using SFC (Daicel CHIRALPAK OZ-H 20×250 mm, 5.0 μm, 50/50 MeOH (0.2% NH₄OH)/CO₂, 45 g/min, 35° C.) to give two enantiomers: (1S,2S)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide and (1R,2R)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide.

First eluting isomer (I-257): ESI-MS (M+H)+: 474.2, ¹H NMR (400 MHz, DMSO) δ 10.60 (s, 1H), 8.29 (s, 1H), 8.19 (s, 1H), 7.85 (s, 1H), 7.61 (s, 1H), 7.37 (d, J=9.3 Hz, 1H), 7.26 (s, 1H), 6.97-6.95 (m, 1H), 6.42-6.41 (m, 1H), 6.29 (d, J=10.3 Hz, 2H), 5.37 (s, 2H), 4.56 (s, 2H), 2.32-2.24 (m, 1H), 2.23-2.16 (m, 1H), 1.94-1.88 (m, 1H), 1.41-1.37 (m, 1H), 1.28-1.20 (m, 1H), 0.92-0.90 (m, 2H), 0.69-0.63 (m, 2H). RT=3.138 min, 100.0% e.e.

Second eluting isomer (I-258): ESI-MS (M+H)+: 474.2, ¹H NMR (400 MHz, DMSO) δ 10.60 (s, 1H), 8.29 (s, 1H), 8.19 (s, 1H), 7.85 (s, 1H), 7.61 (s, 1H), 7.37 (d, J=9.3 Hz, 1H), 7.26 (s, 1H), 6.97-6.95 (m, 1H), 6.42-6.41 (m, 1H), 6.29 (d, J=9.7 Hz, 2H), 5.37 (s, 2H), 4.56 (s, 2H), 2.31-2.25 (m, 1H), 2.23-2.16 (m, 1H), 1.95-1.87 (m, 1H), 1.41-1.37 (m, 1H), 1.28-1.22 (m, 1H), 0.95-0.87 (m, 2H), 0.69-0.63 (m, 2H). RT=4.193 min, 100.0% e.e.

### Examples 259-261

### Synthesis of rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-259)

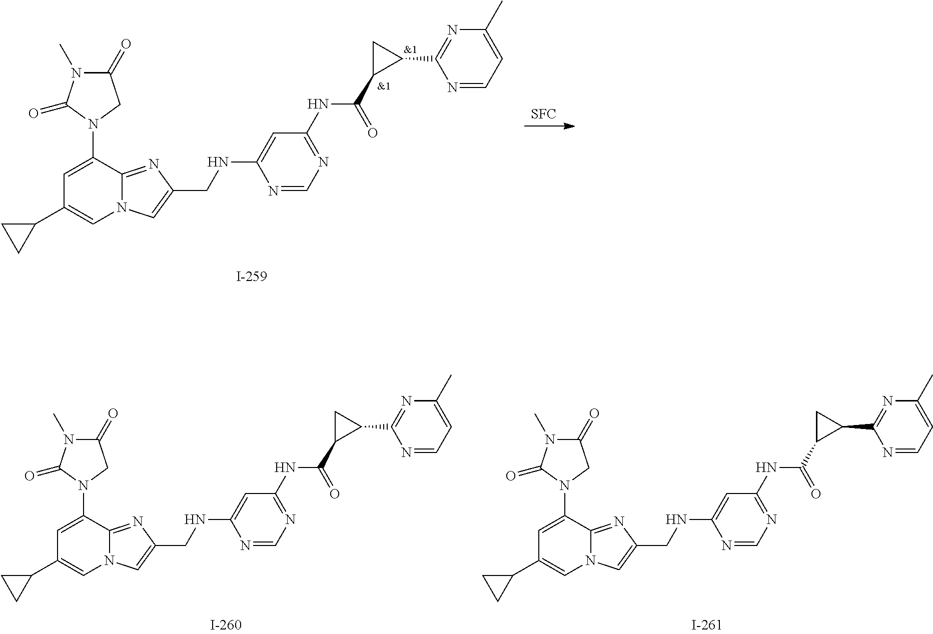

The title compound was prepared in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide from rac-(1R,2R)-2-(4-methylpyrimidin-2-yl)cyclopropane-1-carboxamide and 1-(2-(aminomethyl)-6-cyclopropylimidazo[1,2-a]pyridin-8-yl)-3-methylimidazolidine-2,4-dione and obtained as a mixture of enantiomers. ESI-MS [M+H]+: 553.2, ¹H NMR (400 MHz, DMSO) δ 10.68 (s, 1H), 8.52 (d, J=5.1 Hz, 1H), 8.24 (s, 1H), 8.18 (s, 1H), 7.89 (s, 1H), 7.69 (s, 1H), 7.33-7.31 (m, 2H), 7.21 (d, J=5.1 Hz, 1H), 4.91 (s, 2H), 4.58 (s, 2H), 2.97 (s, 3H), 2.64-2.60 (m, 1H), 2.55-2.52 (m, 1H), 2.41 (s, 3H), 1.97-1.90 (m, 1H), 1.55-1.48 (m, 2H), 0.95-0.91 (m, 2H), 0.66-0.62 (m, 2H).

The mixture was separated using SFC (Daicel CHIRALPAK OJ-H 250 mm×20 mm I.D., 5 m. CO2/MeOH (0.1% DEA)=50/50, 45 g/min, 35° C.) to give two enantiomers: (1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide and (1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide.

First eluting isomer, (1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-260):

ESI-MS [M+H]+: 553.3, ¹H NMR (400 MHz, DMSO) δ 10.68 (s, 1H), 8.53 (d, J=5.1 Hz, 1H), 8.24-8.18 (m, 2H), 7.88 (s, 1H), 7.69 (s, 1H), 7.32 (d, J=9.4 Hz, 2H), 7.21 (d, J=5.1 Hz, 1H), 4.91 (s, 2H), 4.58 (s, 2H), 2.97 (s, 3H), 2.64-2.60 (m, 1H), 2.57-2.53 (m, 1H), 2.41 (s, 3H), 1.98-1.90 (m, 1H), 1.53-1.48 (m, 2H), 0.96-0.91 (m, 2H), 0.68-0.62 (m, 2H). RT=3.21 min, 100% e.e.

Second eluting isomer, (1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (I-261):

ESI-MS [M+H]+: 553.3, ¹H NMR (400 MHz, DMSO) δ 10.68 (s, 1H), 8.52 (d, J=5.1 Hz, 1H), 8.24-8.18 (m, 2H), 7.88 (s, 1H), 7.69 (s, 1H), 7.32 (d, J=9.4 Hz, 2H), 7.21 (d, J=5.1 Hz, 1H), 4.91 (s, 2H), 4.58 (s, 2H), 2.97 (s, 3H), 2.64-2.60 (m, 1H), 2.57-2.53 (m, 1H), 2.41 (s, 3H), 1.98-1.90 (m, 1H), 1.53-1.48 (m, 2H), 0.96-0.91 (m, 2H), 0.68-0.62 (m, 2H). RT=6.47 min, 100% e.e.

### Examples 262-264

### Synthesis of rac-(1S*,2S*)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-262)

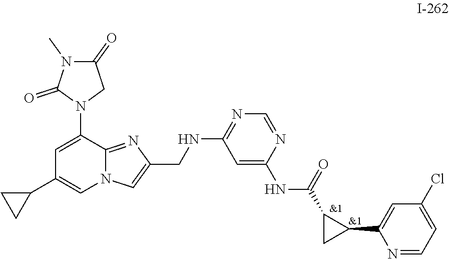 [SMILES: CN1C(=O)CN(c2cc(C3CC3)cn3cc(CNc4cc(NC(=O)[C@H]5C[C@@H]5c5cc(Cl)ccn5)ncn4)nc23)C1=O]

The title compound was prepared in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide from rac-(1R,2R)-2-(4-chloropyridin-2-yl)cyclopropane-1-carboxamide and 1-(2-(aminomethyl)-6-cyclopropylimidazo[1,2-a]pyridin-8-yl)-3-methylimidazolidine-2,4-dione and obtained as a mixture of enantiomers. ESI-MS [M+H]+: 572.1, ¹H NMR (400 MHz, DMSO) δ 10.65 (s, 1H), 8.42 (d, J=5.4 Hz, 1H), 8.24 (d, J=1.1 Hz, 1H), 8.18 (s, 1H), 7.88 (s, 1H), 7.69 (s, 1H), 7.64 (d, J=1.5 Hz, 1H), 7.34 (d, J=2.1 Hz, 2H), 7.31 (d, J=0.7 Hz, 1H), 4.91 (s, 2H), 4.57 (s, 2H), 2.98 (s, 3H), 2.59 (dd, J=13.7, 7.5 Hz, 2H), 1.94 (ddd, J=13.4, 8.3, 5.0 Hz, 1H), 1.56-1.44 (m, 2H), 0.95 (s, 2H), 0.64 (dd, J=5.0, 1.8 Hz, 2H).

The mixture was separated using SFC (Daicel CHIRALPAK AD-H 250 mm×20 mm I.D., 5 μm, CO2/EtOH=74/26, 50 g/min, 35° C.) to give two enantiomers: (1S,2S)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide and (1R,2R)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide.

First eluting isomer (1R,2R)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-263): ESI-MS [M+H]+: 572.2, ¹H NMR (400 MHz, DMSO) δ 10.65 (s, 1H), 8.42 (d, J=5.3 Hz, 1H), 8.24 (s, 1H), 8.18 (s, 1H), 7.89 (s, 1H), 7.69 (s, 1H), 7.64 (s, 1H), 7.39-7.28 (m, 3H), 4.91 (s, 2H), 4.57 (s, 2H), 2.98 (s, 3H), 2.60-2.55 (m, 2H), 1.99-1.88 (m, 1H), 1.55-1.45 (m, 2H), 0.98-0.90 (m, 2H), 0.68-0.59 (m, 2H). RT=0.815 min, 100% e.e.

Second eluting isomer (1S,2S)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide (I-264): ESI-MS [M+H]+: 572.2, ¹H NMR (400 MHz, DMSO) δ 10.65 (s, 1H), 8.42 (d, J=5.2 Hz, 1H), 8.24 (s, 1H), 8.18 (s, 1H), 7.88 (s, 1H), 7.70-7.63 (m, 2H), 7.40-7.25 (m, 3H), 4.91 (s, 2H), 4.56 (s, 2H), 2.98 (s, 3H), 2.62-2.54 (m, 2H), 1.98-1.88 (m, 1H), 1.56-1.44 (m, 2H), 0.98-0.90 (m, 2H), 0.68-0.60 (m, 2H). RT=1.620 min, 100% e.e.

### Examples 265-267

### Synthesis of rac-(1S*,2S*)-2-(4-chloropyridin-2-yl)-N-(6-(((6-cyclopropylimidazo[1,2-a]pyridin-2-yl)methyl)amino)pyrimidin-4-yl)cyclopropane-1-carboxamide (I-265)

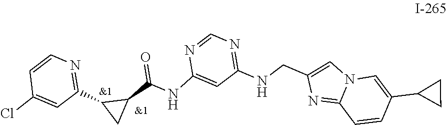 [SMILES: O=C(Nc1cc(NCc2cn3cc(C4CC4)ccc3n2)ncn1)[C@H]1C[C@@H]1c1cc(Cl)ccn1]

The title compound was prepared in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide and obtained as a mixture of enantiomers. ESI-MS [M+H]+: 460.1, ¹H NMR (400 MHz, DMSO) δ=10.64 (s, 1H), 8.42 (d, J=5.4, 1H), 8.30 (s, 1H), 8.19 (s, 1H), 7.90-7.80 (m, 1H), 7.65 (d, J=1.7, 1H), 7.61 (s, 1H), 7.41-7.32 (m, 2H), 7.27 (s, 1H), 6.98-6.93 (m, 1H), 4.56 (s, 2H), 2.63-2.55 (m, 2H), 1.94-1.88 (m, 1H), 1.58-1.41 (m, 2H), 0.94-0.87 (m, 2H), 0.71-0.59 (m, 2H).

The mixture was separated using SFC (Daicel CHIRALPAK OD-H 250 mm×20 mm I.D. 5 μm, CO2/MeOH (0.2% NH4·OH)=50/50, 45 g/min, 35° C.) to give two enantiomers: (1S,2S)-2-(4-chloropyridin-2-yl)-N-(6-(((6-cyclopropylimidazo[1,2-a]pyridin-2-yl)methyl)amino)pyrimidin-4-yl)cyclopropane-1-carboxamide and (1R,2R)-2-(4-chloropyridin-2-yl)-N-(6-(((6-cyclopropylimidazo[1,2-a]pyridin-2-yl)methyl)amino)pyrimidin-4-yl)cyclopropane-1-carboxamide.

First eluting isomer (I-266): ESI-MS [M+H]+: 460.1, ¹H NMR (400 MHz, DMSO) δ 10.64 (s, 1H), 8.42 (d, J=5.4 Hz, 1H), 8.30 (s, 1H), 8.18 (s, 1H), 7.86 (s, 1H), 7.67-7.56 (m, 2H), 7.38-7.32 (m, 2H), 7.27 (s, 1H), 6.98-6.93 (m, 1H), 4.56 (s, 2H), 2.58-2.51 (m, 2H), 1.94-1.87 (m, 1H), 1.55-1.50 (m, 1H), 1.49-1.46 (m, 1H), 0.91-0.88 (m, 2H), 0.68-0.63 (m, 2H). RT=2.462 min, 100% e.e.

Second eluting isomer (I-267): ESI-MS [M+H]+: 460.1, ¹H NMR (400 MHz, DMSO) δ 10.64 (s, 1H), 8.42 (d, J=5.3 Hz, 1H), 8.30 (s, 1H), 8.18 (s, 1H), 7.86 (s, 1H), 7.65-7.61 (m, 2H), 7.36-7.34 (m, 2H), 7.27 (s, 1H), 6.98-6.93 (m, 1H), 4.56 (s, 2H), 2.61-2.55 (m, 2H), 1.93-1.89 (m, 1H), 1.55-1.46 (m, 2H), 0.92-0.89 (m, 2H), 0.67-0.65 (m, 2H). RT=3.034 min, 100% e.e.

### Examples 268-270

### Synthesis of rac-(1S*,2S*)-2-(4-chloropyridin-2-yl)-N-(4-(((6-cyclopropylimidazo[1,2-a]pyridin-2-yl)methyl)amino)pyridin-2-yl)cyclopropane-1-carboxamide (Example I-268)

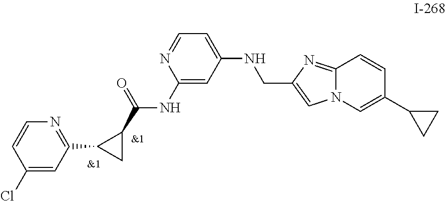 [SMILES: O=C(Nc1cc(NCc2cn3cc(C4CC4)ccc3n2)ccn1)[C@H]1C[C@@H]1c1cc(Cl)ccn1]

The title compound was prepare in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide and obtained as a mixture of enantiomers. ESI-MS [M+H]+: 459.2, ¹H NMR (400 MHz, DMSO) δ 10.34 (s, 1H), 8.41 (d, J=5.3 Hz, 1H), 8.31 (s, 1H), 7.75 (d, J=5.7 Hz, 1H), 7.63 (s, 2H), 7.42 (s, 1H), 7.38 (d, J=9.3 Hz, 1H), 7.35-7.31 (m, 1H), 7.09 (t, J=5.8 Hz, 1H), 6.99-6.94 (m, 1H), 6.34-6.30 (m, 1H), 4.35 (d, J=5.5 Hz, 2H), 2.60-2.53 (m, 2H), 1.94-1.86 (m, 1H), 1.51-1.44 (m, 2H), 0.95-0.87 (m, 2H), 0.71-0.61 (m, 2H).

The mixture was separated using SFC (Daicel CHIRALPAK OD-H 250 mm×20 mm I.D. 5 um, CO2/MeOH (0.1% NH4 OH)=50/50. 45 g/min, 35° C.) to give two enantiomers: (1S,2S)-2-(4-chloropyridin-2-yl)-N-(4-(((6-cyclopropylimidazo[1,2-a]pyridin-2-yl)methyl)amino)pyridin-2-yl)cyclopropane-1-carboxamide and (1R,2R)-2-(4-chloropyridin-2-yl)-N-(4-(((6-cyclopropylimidazo[1,2-a]pyridin-2-yl)methyl)amino)pyridin-2-yl)cyclopropane-1-carboxamide.

First eluting isomer (I-269): ESI-MS [M+H]+: 459.1. ¹H NMR (400 MHz, DMSO) δ 10.35 (s, 1H), 8.41 (d, J=5.3, 1H), 8.31 (s, 1H), 7.75 (d, J=5.8, 1H), 7.63 (d, J=1.9, 2H), 7.45-7.29 (m, 3H), 7.12-7.08 (m, 1H), 6.97 (dd, J=9.3, 1.7, 1H), 6.32 (dd, J=5.8, 2.1, 1H), 4.35 (d, J=5.7, 2H), 2.60-2.53 (m, 2H), 1.94-1.87 (m, 1H), 1.50-1.42 (m, 2H), 0.94-0.87 (m, 2H), 0.69-0.62 (m, 2H). RT=2.20 min, 100.0% e.e

Second eluting isomer (I-270): ESI-MS [M+H]+: 459.2. ¹H NMR (400 MHz, DMSO) δ 10.36 (s, 1H), 8.41 (d, J=5.4, 1H), 8.31 (s, 1H), 7.75 (d, J=5.8, 1H), 7.66-7.59 (m, 2H), 7.44-7.36 (m, 2H), 7.33 (dd, J=5.3, 2.0, 1H), 7.11 (s, 1H), 6.97 (dd, J=9.3, 1.7, 1H), 6.33 (d, J=3.9, 1H), 4.35 (d, J=5.5, 2H), 2.59-2.53 (m, 2H), 1.95-1.85 (m, 1H), 1.52-1.42 (m, 2H), 0.94-0.87 (m, 2H), 0.70-0.62 (m, 2H). RT=3.29 min, 100.0% e.e

### Examples 271-273

### Synthesis of rac-(1S*,2S*)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide (I-271)

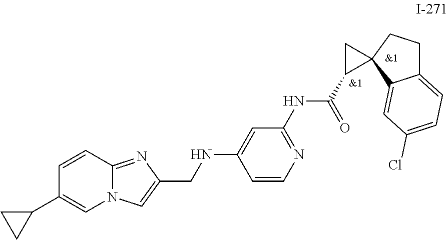 [SMILES: O=C(Nc1cc(NCc2cn3cc(C4CC4)ccc3n2)ccn1)[C@@H]1C[C@]12CCc1ccc(Cl)cc12]

The title compound was prepared in a similar manner to (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide and obtained as a mixture of enantiomers. ESI-MS [M+H]+: 484.2. ¹H NMR (400 MHz, DMSO) δ 10.23 (s, 1H), 8.30 (s, 1H), 7.73 (d, J=5.8 Hz, 1H), 7.64 (s, 1H), 7.44 (s, 1H), 7.38 (d, J=9.3 Hz, 1H), 7.28-7.05 (m, 3H), 6.97 (dd, J=9.3, 1.7 Hz, 1H), 6.88 (d, J=1.8 Hz, 1H), 6.31 (dd, J=5.8, 2.0 Hz, 1H), 4.36 (d, J=5.6 Hz, 2H), 3.03-2.78 (m, 2H), 2.44-2.42 (m, 1H), 2.24-2.11 (m, 2H), 1.98-1.84 (m, 1H), 1.60-1.40 (m, 2H), 0.98-0.85 (m, 2H), 0.75-0.59 (m, 2H).

The mixture was separated using SFC (Daicel CHIRALPAK OD-H, 250 mm×20 mm I.D. CO₂/MeOH (0.1% NH4OH)=50/50. 50 g/min, 35° C.) to give two enantiomers: (1S,2S)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide and (1R,2R)-6′-chloro-N-(4-(((6-cyclo propylimidazo[1,2-a]pyridin-2-yl)methyl)amino)pyridin-2-yl)-2′,3′-dihydrospiro[cyclopropane-1,1′-indene]-2-carboxamide. RT=2.15 min, 100.0% e.e

First eluting isomer (I-272): ESI-MS [M+H]+: 484.1, ¹H NMR (400 MHz, DMSO) δ 10.22 (s, 1H), 8.30 (s, 1H), 7.73 (d, J=5.8 Hz, 1H), 7.64 (s, 1H), 7.44 (s, 1H), 7.38 (d, J=9.3 Hz, 1H), 7.25-7.08 (m, 3H), 6.97 (dd, J=9.3, 1.7 Hz, 1H), 6.88 (d, J=1.8 Hz, 1H), 6.31 (d, J=3.9 Hz, 1H), 4.35 (d, J=5.7 Hz, 2H), 3.00-2.79 (m, 2H), 2.48-2.40 (m, 1H), 2.23-2.09 (m, 2H), 1.95-1.86 (m, 1H), 1.58-1.42 (m, 2H), 0.97-0.86 (m, 2H), 0.72-0.60 (m, 2H). RT=2.15 min, 100.0% e.e

Second eluting isomer (I-273): ESI-MS [M+H]+: 484.1, NMR (400 MHz, DMSO) δ 10.21 (s, 1H), 8.30 (s, 1H), 7.73 (d, J=5.8 Hz, 1H), 7.64 (s, 1H), 7.45 (s, 1H), 7.38 (d, J=9.3 Hz, 1H), 7.25-7.04 (m, 3H), 6.97 (dd, J=9.3, 1.7 Hz, 1H), 6.88 (d, J=1.7 Hz, 1H), 6.31 (d, J=4.0 Hz, 1H), 4.35 (d, J=5.6 Hz, 2H), 2.97-2.80 (m, 2H), 2.47-2.40 (m, 1H), 2.21-2.08 (m, 2H), 1.97-1.85 (m, 1H), 1.59-1.43 (m, 2H), 0.97-0.86 (m, 2H), 0.75-0.60 (m, 2H). RT=3.52 min, 100.0% e.e

### Example 274

### Synthesis of (1S,2S)-2-(3-chlorophenyl)-N-(6-(((6-cyclopropyl-8-(N-methylacetamido)imidazo[1,2-a]pyridin-2-yl)methyl)amino)pyrimidin-4-yl)cyclopropane-1-carboxamide (I-274)

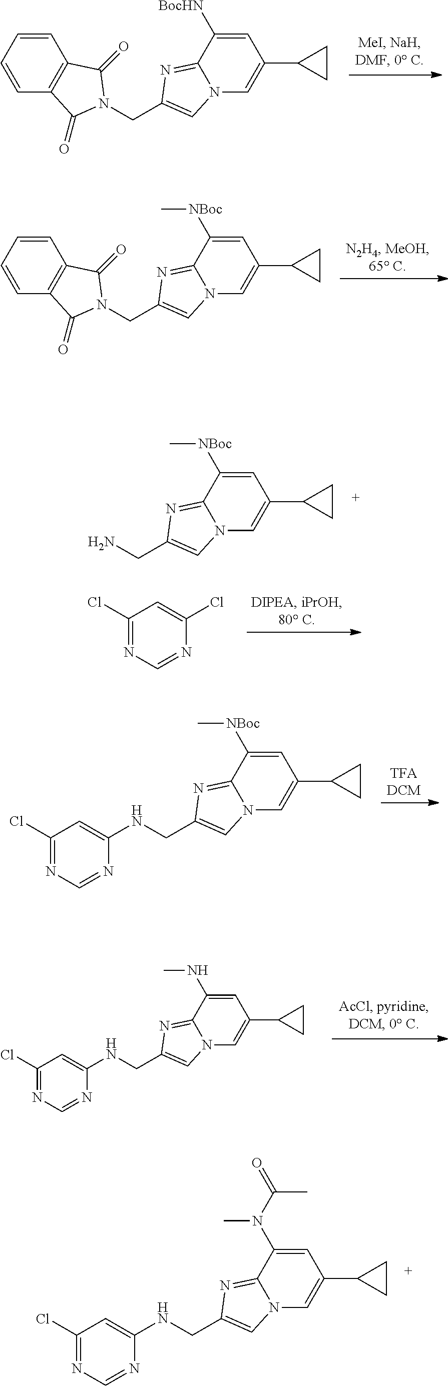

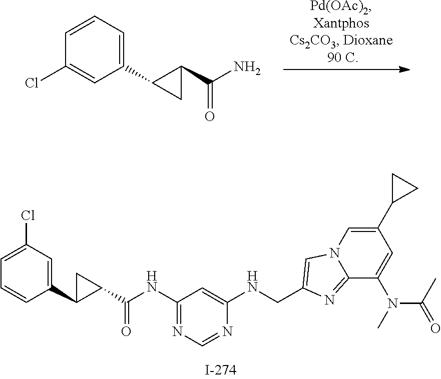 [SMILES: NC(=O)[C@H]1C[C@@H]1c1cccc(Cl)c1 ; "Pd(OAc)2,XantphosCs2CO3, Dioxane 90 C." ; CC(=O)N(C)c1cc(C2CC2)cn2cc(CNc3cc(NC(=O)[C@H]4C[C@@H]4c4cccc(Cl)c4)ncn3)nc12]

Synthesis of tert-butyl (6-cyclopropyl-2-((1,3-dioxoisoindolin-2-yl)methyl)imidazo[1,2-a]pyridin-8-yl)(methyl)carbamate. A solution of tert-butyl (6-cyclopropyl-2-((1,3-dioxoisoindolin-2-yl)methyl)imidazo[1,2-a]pyridin-8-yl)carbamate (500 mg, 1.2 mmol) in DMF (10 mL) was cooled to 0° C. NaH (60% in mineral oil, 56 mg, 1.4 mmol) was added and the reaction mixture was stirred at 0° C. for 30 min. Mel (86 μL, 1.4 mmol) was added and the reaction mixture was stirred at 0° C. for 2 h. The reaction mixture was allowed to warm to room temperature, quenched with water (10 mL), and extracted with EtOAc (3×20 mL). The combined organics were passed through a hydrophobic frit and concentrated in vacuo. The residue was purified by column chromatography on silica gel, eluting with a gradient of 0-100% EtOAc in cyclohexane to give the title compound (350 mg, 68%). ¹H NMR (400 MHz, CDCl₃) δ 7.87 (dd, J=3.0, 5.6 Hz, 2H), 7.72 (dd, J=3.0, 5.6 Hz, 2H), 7.68 (s, 1H), 7.41 (s, 1H), 6.81 (s, 1H), 5.05 (s, 2H), 3.34 (s, 3H), 1.88-1.80 (m, 1H), 1.37 (s, 9H), 0.96-0.89 (m, 2H), 0.65-0.59 (m, 2H).

Synthesis of tert-butyl (2-(aminomethyl)-6-cyclopropylimidazo[1,2-a]pyridin-8-yl)(methyl)carbamate. Using a similar procedure to that for 3-(2-(aminomethyl)-6-cyclopropylimidazo[1,2-a]pyridin-8-yl)oxetan-3-ol, the title compound (260 mg, quant.) was synthesized from tert-butyl (6-cyclopropyl-2-((1,3-dioxoisoindolin-2-yl)methyl)imidazo[1,2-a]pyridin-8-yl)(methyl)carbamate using hydrazine hydrate. ESI-MS (M+H)+: 317.4

Synthesis of tert-butyl (2-(((6-chloropyrimidin-4-yl)amino)methyl)-6-cyclopropylimidazo[1,2-a]pyridin-8-yl)(methyl)carbamate. Using a similar procedure to that used for 1-(2-(((6-chloropyrimidin-4-yl)amino)methyl)-6-cyclopropylimidazo[1,2-a]pyridin-8-yl)pyrrolidin-2-one, the title compound (160 mg, 40%) was made from 2,4-dichloropyrimidine and tert-butyl (2-(aminomethyl)-6-cyclopropylimidazo[1,2-a]pyridin-8-yl)(methyl)carbamate. ESI-MS (M+H)+: 429.3

Synthesis of 2-(((6-chloropyrimidin-4-yl)amino)methyl)-6-cyclopropyl-N-methylimidazo[1,2-a]pyridin-8-amine. Trifluoroacetic acid (0.11 mL, 1.49 mmol) was added to a solution of tert-butyl (2-(((6-chloropyrimidin-4-yl)amino)methyl)-6-cyclopropylimidazo[1,2-a]pyridin-8-yl)(methyl)carbamate (160 mg, 0.37 mmol) in DCM (5.0 mL), and the reaction mixture was stirred at room temperature for 2 h. The reaction mixture was quenched with NaHCO₃ (sat·aq·, 35 mL) and extracted with DCM (2×50 mL). The combined organics were passed through a hydrophobic frit and concentrated in vacuo to give the title compound (126 mg, quant %). ESI-MS (M+H)+: 329.3.

Synthesis of N-(2-(((6-chloropyrimidin-4-yl)amino)methyl)-6-cyclopropylimidazo[1,2-a]pyridin-8-yl)-N-methylacetamide. Acetyl chloride (26 μL, 0.36 mmol) was added to a solution of 2-(((6-chloropyrimidin-4-yl)amino)methyl)-6-cyclopropyl-N-methylimidazo[1,2-a]pyridin-8-amine (120 mg, 0.36 mmol) and pyridine (0.088 mL, 1.09 mmol) in DCM (1.0 mL) at 0° C., and the reaction mixture was stirred at 0° C. for 2 h. The reaction mixture was allowed to warm to room temperature, quenched with water (5 mL), and extracted with DCM (3×15 mL). The combined organics were passed through a hydrophobic frit and concentrated in vacuo. The residue was purified by column chromatography on silica gel, eluting with a gradient of 0-10% (7N NH₃ in MeOH) in DCM to give the title compound which was used directly in the next reaction. ESI-MS (M+H)+: 371.3.

Synthesis of (1S,2S)-2-(3-chlorophenyl)-N-(6-(((6-cyclopropyl-8-(N-methylacetamido)imidazo[1,2-a]pyridin-2-yl)methyl)amino)pyrimidin-4-yl)cyclopropane-1-carboxamide (I-274). Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using (1S,2S)-2-(3-chlorophenyl)cyclopropane-1-carboxamide and N-(2-(((6-chloropyrimidin-4-yl)amino)methyl)-6-cyclopropylimidazo[1,2-a]pyridin-8-yl)-N-methylacetamide then purified by column chromatography on silica gel, eluting with a gradient of 0-10% (7N NH₃ in MeOH) in DCM (0.63 mg). ESI-MS (M+H)+: 530.4, ¹H NMR (400 MHz, DMSO) δ 10.64 (s, 1H), 8.38 (s, 1H), 8.25 (s, 1H), 7.99-7.92 (m, 1H), 7.74 (s, 1H), 7.40-7.29 (m, 4H), 7.22-7.18 (m, 1H), 7.10 (s, 1H), 4.63-4.61 (m, 2H), 3.25 (br s, 3H), 2.47-2.37 (m, 2H), 2.02-1.94 (m, 1H), 1.84 (br s, 3H), 1.54-1.42 (m, 2H), 1.01-0.95 (m, 2H), 0.79-0.73 (m, 2H).

### Example 275

### Synthesis of (1S,2S)—N-(6-(((8-acetamido-6-cyclopropylimidazo[1,2-a]pyridin-2-yl)methyl)amino)pyrimidin-4-yl)-2-(3-chlorophenyl)cyclopropane-1-carboxamide (I-275)

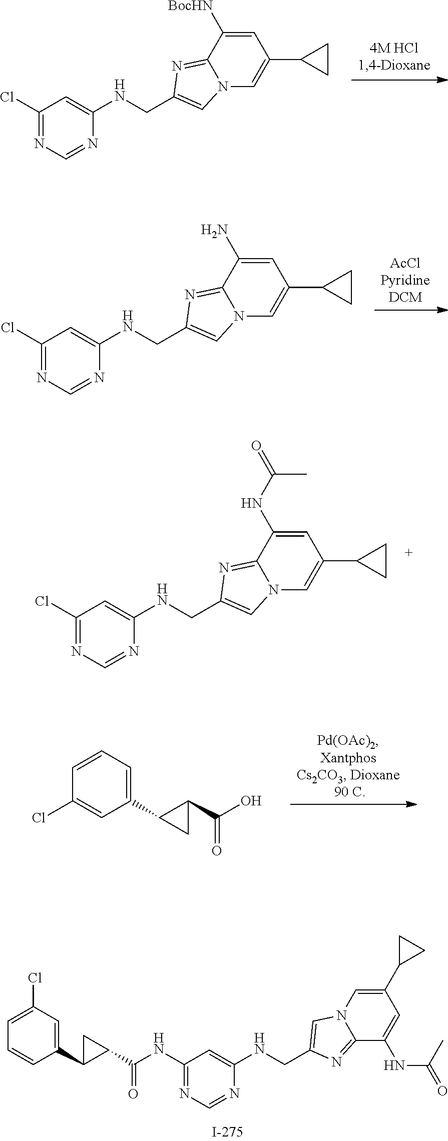

Synthesis of 2-(((6-chloropyrimidin-4-yl)amino)methyl)-6-cyclopropylimidazo[1,2-a]pyridin-8-amine. A solution of HCl (4M in 1,4-Dioxane, 0.16 mL) was added to a solution of tert-butyl (2-(((6-chloropyrimidin-4-yl)amino)methyl)-6-cyclopropylimidazo[1,2-a]pyridin-8-yl)carbamate (175 mg, 0.42 mmol) in 1,4-dioxane (5.0 mL), followed by a few drops of water. The resulting solution was stirred for 2 h. Further HCl (4M in 1,4-Dioxane, 0.5 mL) was added, then then mixture was stirred for 18 h at room temperature, then for 2 h at reflux. NaHCO₃ (sat·aq·, 10 mL) was added, and then the mixture was extracted with EtOAc (3×10 mL). The combined aqueous layers were passed through a hydrophobic frit, then concentrated in vacuo. The residue was purified by column chromatography on silica gel, eluting with a gradient of 0-10% (7N NH₃ in MeOH) in DCM to give the title compound (61 mg, 46%). ESI-MS (M+H)+: 315.1

Synthesis of N-(2-(((6-chloropyrimidin-4-yl)amino)methyl)-6-cyclopropylimidazo[1,2-a]pyridin-8-yl)acetamide. Using a similar procedure to that used for the synthesis of N-(2-(((6-chloropyrimidin-4-yl)amino)methyl)-6-cyclopropylimidazo[1,2-a]pyridin-8-yl)-N-methylacetamide, the title compound was prepared from 2-(((6-chloropyrimidin-4-yl)amino)methyl)-6-cyclopropylimidazo[1,2-a]pyridin-8-amine (35 mg, 55%). ESI-MS (M+H)+: 357.1.

Synthesis of (1S,2S)—N-(6-(((8-acetamido-6-cyclopropylimidazo[1,2-a]pyridin-2-yl)methyl)amino)pyrimidin-4-yl)-2-(3-chlorophenyl)cyclopropane-1-carboxamide (I-275). Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using (1S,2S)-2-(3-chlorophenyl)cyclopropane-1-carboxamide and N-(2-(((6-chloropyrimidin-4-yl)amino)methyl)-6-cyclopropylimidazo[1,2-a]pyridin-8-yl)acetamide and purified by column chromatography on silica gel, eluting with a gradient of 0-20% (7N NH₃ in MeOH) in DCM followed by preparative HPLC (7.1 mg, 14%). ESI-MS (M+H)+: 516.2, ¹H NMR (400 MHz, DMSO) δ 10.63 (s, 1H), 9.89 (s, 1H), 8.23-8.23 (m, 1H), 8.05 (s, 1H), 7.91-7.86 (m, 1H), 7.79-7.76 (m, 1H), 7.65 (s, 1H), 7.37-7.28 (m, 4H), 7.18 (d, J=7.6 Hz, 1H), 4.63-4.63 (m, 2H), 2.47-2.37 (m, 2H), 2.22 (s, 3H), 1.96-1.88 (m, 1H), 1.53-1.39 (m, 2H), 0.96-0.90 (m, 2H), 0.66-0.60 (m, 2H).

### Example 276

### Synthesis of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-(dimethylamino)ethoxy)pyrimidin-4-yl)-2-(4-methylpyrimidin-2-yl)cyclopropane-1-carboxamide (I-276)

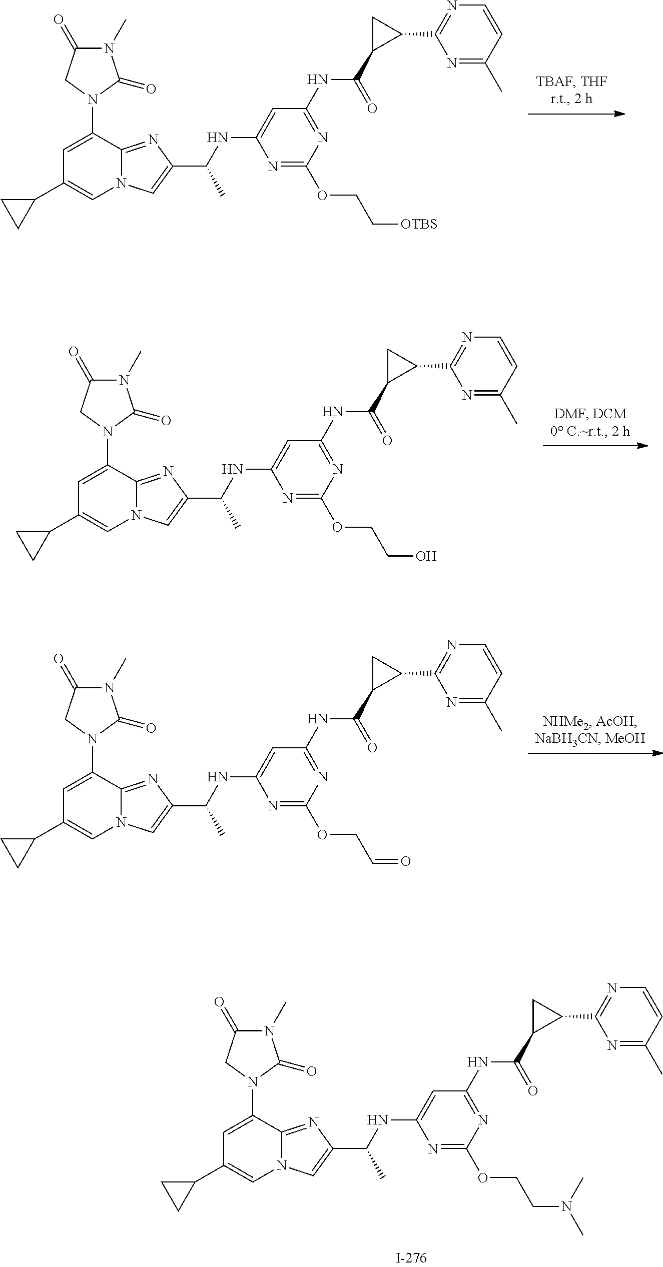

Synthesis of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethoxy)pyrimidin-4-yl)-2-(4-methylpyrimidin-2-yl)cyclopropane-1-carboxamide. To a solution of (1S,2S)—N-(2-(2-((tert-butyldimethylsilyl)oxy)ethoxy)-6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (170 Mg, 0.23 mmol) in THF (10 mL) was added TBAF (0.7 mL, 1M solution in THF, 0.7 mmol). The resulting reaction was stirred at room temperature for 2 h. The reaction was concentrated in vacuo to give the crude, which was purified with preparative TLC (eluent: DCM/MeOH=10/1) to give (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethoxy)pyrimidin-4-yl)-2-(4-methylpyrimidin-2-yl)cyclopropane-1-carboxamide (80 mg, 56%) as a yellow solid. ESI-MS [M+H]+: 627.2.

Synthesis of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-oxoethoxy)pyrimidin-4-yl)-2-(4-methylpyrimidin-2-yl)cyclopropane-1-carboxamide. To a solution of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethoxy)pyrimidin-4-yl)-2-(4-methylpyrimidin-2-yl)cyclopropane-1-carboxamide (80 mg, 0.128 mmol) in DCM (10 mL) was added Dess-Martin Periodinane (108 mg, 0.255 mmol). The resulting reaction was stirred at room temperature for 2 h. The reaction was quenched with saturated aqueous NaHCO₃ (30 mL) and extracted with DCM (3×30 mL). The combined organic layers were washed with brine (30 mL), dried over Na2SO4, and concentrated in vacuo to give the crude, which was purified with preparative TLC (eluent: DCM/MeOH=15/1) to give (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-oxoethoxy)pyrimidin-4-yl)-2-(4-methylpyrimidin-2-yl)cyclopropane-1-carboxamide (30 mg, 37.5%) as a yellow solid. ESI-MS [M+H]+: 625.2.

Synthesis of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-(dimethylamino)ethoxy)pyrimidin-4-yl)-2-(4-methylpyrimidin-2-yl)cyclopropane-1-carboxamide (I-276). A solution of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-oxoethoxy)pyrimidin-4-yl)-2-(4-methylpyrimidin-2-yl)cyclopropane-1-carboxamide (30 mg, 0.048 mmol), NHMe₂ (22 mg, 0.48 mmol), and AcOH (1 drop) in MeOH (3 mL) was stirred at room temperature for 30 min. Then NaBH₃CN (10 mg, 0.144 mmol) was added to the mixture, which was then stirred at room temperature for 3 h. The reaction was quenched with saturated aqueous NaHCO₃ (20 mL) and extracted with DCM (3×20 mL). The combined organic layers were washed with brine (30 mL), dried over Na₂SO₄, and concentrated in vacuo to give the crude, which was purified with preparative TLC (eluent: DCM/MeOH=10/1) to give (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-(dimethylamino)ethoxy)pyrimidin-4-yl)-2-(4-methylpyrimidin-2-yl)cyclopropane-1-carboxamide (12 mg, 38.8%) as a white solid. ESI-MS (M+H)+: 654.3, ¹H NMR (400 MHz, DMSO) δ 10.52 (s, 1H), 8.52 (d, J=5.1 Hz, 1H), 8.23 (d, J=1.1 Hz, 1H), 7.83 (s, 1H), 7.64 (s, 1H), 7.33 (d, J=1.3 Hz, 1H), 7.21 (d, J=5.1 Hz, 1H), 6.98 (s, 1H), 5.31 (s, 1H), 4.93 (s, 2H), 4.26-4.17 (m, 2H), 2.98 (s, 3H), 2.64-2.58 (m, 1H), 2.56-2.51 (m, 2H), 2.48-2.45 (m, 1H), 2.41 (s, 3H), 2.15 (s, 6H), 1.96-1.89 (m, 1H), 1.56-1.46 (m, 5H), 0.96-0.91 (m, 2H), 0.67-0.61 (m, 2H).

### Example 277

### Synthesis of rac-(1S*,2S*)-2-(2-amino-6-methoxypyridin-4-yl)-N-(6-(((6-cyclopropylimidazo[1,2-a]pyridin-2-yl)methyl)amino)pyrimidin-4-yl)cyclopropane-1-carboxamide (I-277)

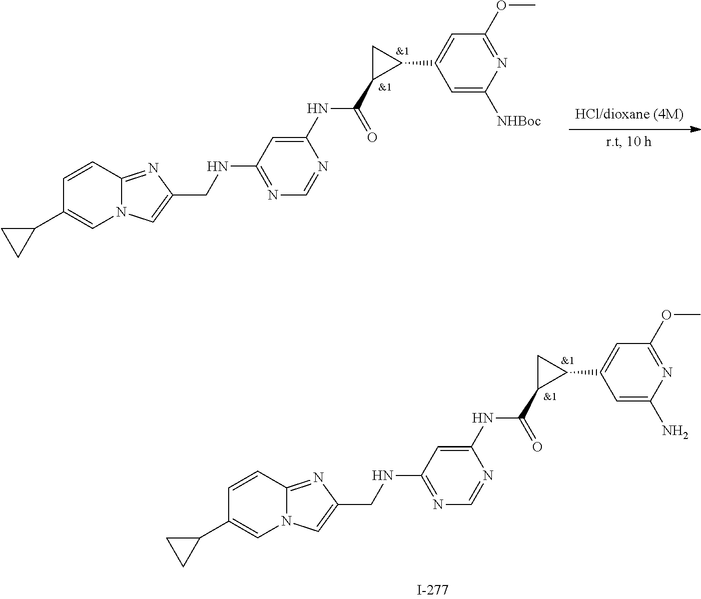 [SMILES: COc1cc([C@H]2C[C@@H]2C(=O)Nc2cc(NCc3cn4cc(C5CC5)ccc4n3)ncn2)cc(NC(=O)OC(C)(C)C)n1 ; "HCl/dioxane (4M)" ; "r.t, 10 h" ; COc1cc([C@H]2C[C@@H]2C(=O)Nc2cc(NCc3cn4cc(C5CC5)ccc4n3)ncn2)cc(N)n1]

To a solution of rac-tert-butyl (4-((1S*,2S*)-2-((6-(((6-cyclopropylimidazo[1,2-a]pyridin-2-yl)methyl)amino)pyrimidin-4-yl)carbamoyl)cyclopropyl)-6-methoxypyridin-2-yl)carbamate (78 mg, 0.137 mmol) in MeOH (2 mL) was added HCl (4 M solution in dioxane, 2 mL). The resulting mixture was stirred at room temperature for 10 h. The reaction mixture was diluted in water (40 mL), adjusted to pH=9˜10 with NaHCO₃ aqueous (10 mL), and extracted with DCM (2×30 ml). The combined organics were washed with brine (60 mL), dried over Na₂SO₄, concentrated, and dried in vacuo to give the title compound as a mixture of enantiomers. ESI-MS (M+H)+: 471.3, ¹H NMR (400 MHz, MeOD) δ 8.20-8.14 (m, 2H), 7.61 (s, 1H), 7.37 (d, J=9.3 Hz, 1H), 7.32 (d, J=0.8 Hz, 1H), 7.06 (dd, J=9.4, 1.7 Hz, 1H), 5.93 (d, J=0.9 Hz, 1H), 5.79 (d, J=0.9 Hz, 1H), 4.64 (s, 2H), 3.77 (s, 3H), 2.32-2.26 (m, 1H), 2.23-2.17 (m, 1H), 1.96-1.89 (m, 1H), 1.58-1.52 (m, 1H), 1.40-1.33 (m, 1H), 0.99-0.94 (m, 2H), 0.73-0.67 (m, 2H).

### Example 278

### Synthesis of rac-(1S*,2S*)-2-(4-amino-3-chlorophenyl)-N-(6-(((6-cyclopropylimidazo[1,2-a]pyridin-2-yl)methyl)amino)pyrimidin-4-yl)cyclopropane-1-carboxamide (I-278)

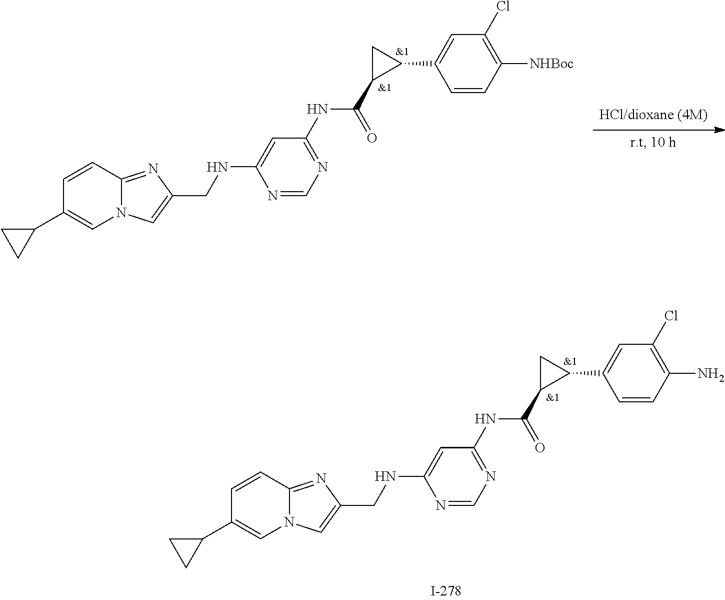 [SMILES: CC(C)(C)OC(=O)Nc1ccc([C@H]2C[C@@H]2C(=O)Nc2cc(NCc3cn4cc(C5CC5)ccc4n3)ncn2)cc1Cl ; "HCl/dioxane (4M)" ; "r.t, 10 h" ; Nc1ccc([C@H]2C[C@@H]2C(=O)Nc2cc(NCc3cn4cc(C5CC5)ccc4n3)ncn2)cc1Cl]

The title compound was prepared in a similar manner to rac-(1S*,2S*)-2-(2-amino-6-methoxypyridin-4-yl)-N-(6-(((6-cyclopropylimidazo[1,2-a]pyridin-2-yl)methyl)amino)pyrimidin-4-yl)cyclopropane-1-carboxamide. ESI-MS (M+H)+: 474.2, ¹H NMR (400 MHz, DMSO) δ 10.55 (s, 1H), 8.31-8.27 (m, 1H), 8.18 (d, J=3.6 Hz, 1H), 7.83 (s, 1H), 7.61 (s, 1H), 7.37 (d, J=9.3 Hz, 1H), 7.26 (s, 1H), 7.00 (d, J=1.9 Hz, 1H), 6.97 (dd, J=9.3, 1.7 Hz, 1H), 6.85 (dd, J=8.3, 1.9 Hz, 1H), 6.71 (d, J=8.3 Hz, 1H), 5.20 (s, 2H), 4.56 (s, 2H), 2.26-2.15 (m, 2H), 1.95-1.87 (m, 1H), 1.39-1.32 (m, 1H), 1.27-1.21 (m, 1H), 0.94-0.88 (m, 2H), 0.69-0.63 (m, 2H).

### Example 279

### Synthesis of rac-(1S*,2S*)-2-(2-amino-5-chlorophenyl)-N-(6-(((6-cyclopropylimidazo[1,2-a]pyridin-2-yl)methylamino)pyrimidin-4-yl)cyclopropane-1-carboxamide (I-279)

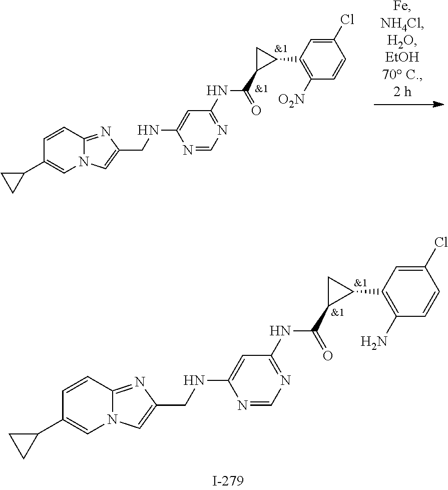 [SMILES: O=C(Nc1cc(NCc2cn3cc(C4CC4)ccc3n2)ncn1)[C@H]1C[C@@H]1c1cc(Cl)ccc1[N+](=O)[O-] ; "Fe,NH4Cl,H2O,EtOH70° C.,2 h" ; Nc1ccc(Cl)cc1[C@H]1C[C@@H]1C(=O)Nc1cc(NCc2cn3cc(C4CC4)ccc3n2)ncn1]

A mixture of rac-(1S*,2S*)-2-(5-chloro-2-nitrophenyl)-N-(6-(((6-cyclopropylimidazo[1,2-a]pyridin-2-yl)methyl)amino)pyrimidin-4-yl)cyclopropane-1-carboxamide (70 mg, 0.14 mmol), NH₄Cl (60 mg, 1.12 mmol), and Fe (39 mg, 0.7 mmol) in water (0.5 ml) and EtOH (2 ml) were stirred at 70° C. for 2 h. After the reaction was completed, water (10 ml) was added, and the mixture was extracted with DCM (3×10 ml), washed with brine (2×10 ml), dried over Na₂SO₄, and concentrated to give the crude, which was purified by preparative TLC (DCM:MeOH=15:1) to afford rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide (30 mg, 45%) as a yellow solid. ESI-MS (M+H)+: 474.2, ¹H NMR (400 MHz, DMSO) δ 10.67 (s, 1H), 8.32 (s, 1H), 8.21 (s, 1H), 7.87 (s, 1H), 7.63 (s, 1H), 7.39 (d, J=9.3 Hz, 1H), 7.29 (s, 1H), 7.02-6.94 (m, 3H), 6.65-6.62 (m, 1H), 5.11 (s, 2H), 4.58 (s, 2H), 2.32-2.26 (m, 1H), 2.18-2.10 (m, 1H), 1.97-1.86 (m, 1H), 1.46-1.38 (m, 1H), 1.11-1.02 (m, 1H), 0.95-0.87 (m, 2H), 0.70-0.62 (m, 2H).

### Example 280

### Synthesis of rac-(1S*,2S*)-2-(2-amino-3-chlorophenyl)-N-(6-(((6-cyclopropylimidazo[1,2-a]pyridin-2-yl)methyl)amino)pyrimidin-4-yl)cyclopropane-1-carboxamide (I-280)

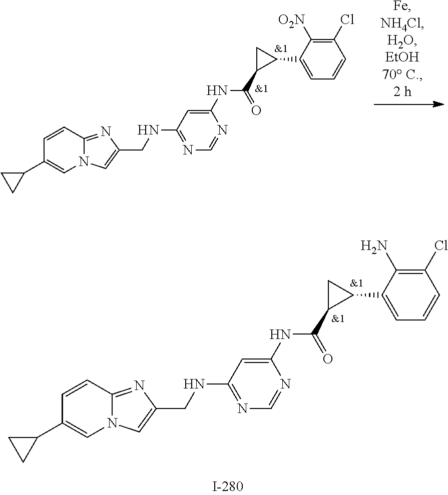 [SMILES: O=C(Nc1cc(NCc2cn3cc(C4CC4)ccc3n2)ncn1)[C@H]1C[C@@H]1c1cccc(Cl)c1[N+](=O)[O-] ; "Fe,NH4Cl,H2O,EtOH70° C.,2 h" ; Nc1c(Cl)cccc1[C@H]1C[C@@H]1C(=O)Nc1cc(NCc2cn3cc(C4CC4)ccc3n2)ncn1]

The title compound was prepared in a similar manner to rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide. ESI-MS (M+H)+: 474.2, ¹H NMR (400 MHz, DMSO) δ 10.67 (s, 1H), 8.32 (s, 1H), 8.21 (s, 1H), 7.87 (s, 1H), 7.64 (s, 1H), 7.40 (d, J=9.3 Hz, 1H), 7.31 (s, 1H), 7.16-7.11 (m, 1H), 7.04-6.90 (m, 2H), 6.55 (t, J=7.8 Hz, 1H), 5.04 (s, 2H), 4.58 (s, 2H), 2.30-2.20 (m, 2H), 1.97-1.86 (m, 1H), 1.50-1.39 (m, 1H), 1.19-1.07 (m, 1H), 0.97-0.87 (m, 2H), 0.71-0.61 (m, 2H).

### Example 281

### Synthesis of (1S,2S)-2-(3-chlorophenyl)-N-(6-(((6-cyclopropyl-8-(1-hydroxyethyl)imidazo[1,2-a]pyridin-2-yl)methyl)amino)pyrimidin-4-yl)cyclopropane-1-carboxamide (I-281)

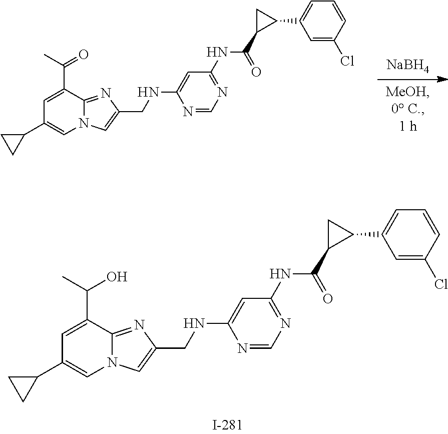 [SMILES: CC(=O)c1cc(C2CC2)cn2cc(CNc3cc(NC(=O)[C@H]4C[C@@H]4c4cccc(Cl)c4)ncn3)nc12 ; "MeOH,0° C.,1 h" ; "NaBH4" ; CC(O)c1cc(C2CC2)cn2cc(CNc3cc(NC(=O)[C@H]4C[C@@H]4c4cccc(Cl)c4)ncn3)nc12]

To a solution of (1S,2S)—N-(6-(((8-acetyl-6-cyclopropylimidazo[1,2-a]pyridin-2-yl)methyl)amino)pyrimidin-4-yl)-2-(3-chlorophenyl)cyclopropane-1-carboxamide (25 mg, 0.05 mmol) in MeOH (5 mL) was added NaBH₄ (0.95 mg, 0.025 mmol) slowly under stirring at 0° C. The mixture was further stirred for 1 h at 0° C., and then stirred at room temperature for another 1 h. The mixture was concentrated in vacuo and purified by preparative HPLC to give (1S,2S)-2-(3-chlorophenyl)-N-(6-(((6-cyclopropyl-8-(1-hydroxyethyl)imidazo[1,2-a]pyridin-2-yl)methyl)amino)pyrimidin-4-yl)cyclopropane-1-carboxamide (4 mg, 16%) as a white solid. ESI-MS (M+H)+: 503.2, ¹H NMR (400 MHz, DMSO) δ 10.60 (s, 1H), 8.40 (s, 1H), 8.18 (d, J=9.6 Hz, 2H), 7.88 (s, 1H), 7.59 (s, 1H), 7.32-7.25 (m, 4H), 7.16-7.14 (m, 1H), 7.00 (s, 1H), 5.33 (s, 1H), 5.20-5.16 (m, 1H), 4.57 (s, 2H), 2.42-2.38 (m, 2H), 1.94-1.90 (m, 1H), 1.46-1.41 (m, 5H), 0.93-0.90 (m, 2H), 0.67-0.63 (m, 2H).

### Example 282

### Synthesis of (1S,2S)-2-(3-chlorophenyl)-N-(4-((2R,4S)-2-(6-cyclopropylimidazo[1,2-a]pyridin-2-yl)-4-hydroxypyrrolidin-1-yl)pyridin-2-yl)cyclopropane-1-carboxamide (I-282)

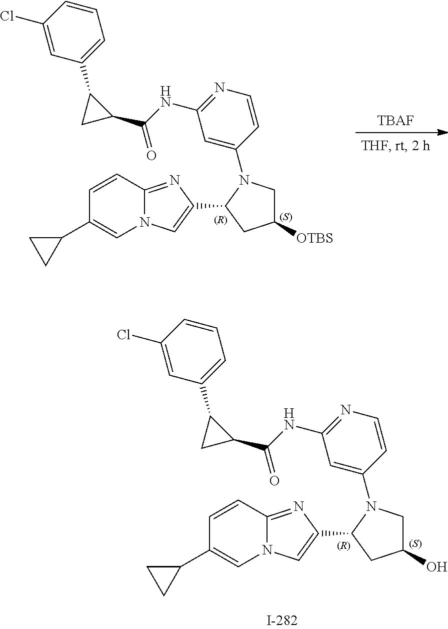

To a solution of (1S,2S)—N-(4-((2R,4S)-4-((tert-butyldimethylsilyl)oxy)-2-(6-cyclopropylimidazo[1,2-a]pyridin-2-yl)pyrrolidin-1-yl)pyridin-2-yl)-2-(3-chlorophenyl)cyclopropane-1-carboxamide (10 mg, 0.016 mmol) in THF (4 mL) was added TBAF (1.0M, 0.03 mL). After stirring the mixture at room temperature for 2 h, water (10 mL) was added, and the mixture was then extracted with EtOAc (3×30 mL). The combined organic layers were washed with brine, dried over Na₂SO₄, filtered, and concentrated in vacuo to give the crude, which was purified by preparative TLC to give (1S,2S)-2-(3-chlorophenyl)-N-(4-((2R,4S)-2-(6-cyclopropylimidazo[1,2-a]pyridin-2-yl)-4-hydroxypyrrolidin-1-yl)pyridin-2-yl)cyclopropane-1-carboxamide (4.7 mg, 57%) as a white solid. ESI-MS [M+H]+: 514.2, ¹H NMR (400 MHz, MeOD) δ 8.10 (s, 1H), 7.73 (d, J=6.0 Hz, 1H), 7.55 (s, 1H), 7.35-7.31 (m, 2H), 7.29-7.22 (m, 1H), 7.21-7.16 (m, 2H), 7.13-7.03 (m, 2H), 6.28 (d, J=4.8 Hz, 1H), 5.14-5.10 (m, 1H), 4.92-4.87 (m, 1H), 4.63-4.60 (m, 1H), 4.00-3.92 (m, 2H), 3.50-3.45 (m, 1H), 2.48-2.40 (m, 2H), 2.39-2.30 (m, 1H), 2.24-2.17 (m, 1H), 1.99-1.87 (m, 1H), 1.64-1.55 (m, 1H), 1.44-1.37 (m, 1H), 1.06-0.85 (m, 2H), 0.78-0.67 (m, 2H).

### Example 283

### Synthesis of (1S,2S)-2-(3-chlorophenyl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide (I-283)

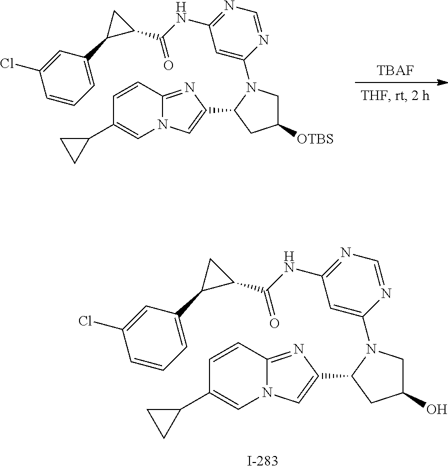 [SMILES: CC(C)(C)[Si](C)(C)O[C@H]1C[C@H](c2cn3cc(C4CC4)ccc3n2)N(c2cc(NC(=O)[C@H]3C[C@@H]3c3cccc(Cl)c3)ncn2)C1 ; "TBAF" ; "THF, rt, 2 h" ; O=C(Nc1cc(N2C[C@@H](O)C[C@@H]2c2cn3cc(C4CC4)ccc3n2)ncn1)[C@H]1C[C@@H]1c1cccc(Cl)c1]

The title compound was prepared in a similar manner to (1S,2S)-2-(3-chlorophenyl)-N-(4-((2R,4S)-2-(6-cyclopropylimidazo[1,2-a]pyridin-2-yl)-4-hydroxypyrrolidin-1-yl)pyridin-2-yl)cyclopropane-1-carboxamide. ESI-MS [M+H]+: 515.2, ¹H NMR (400 MHz, MeOD) δ 8.14 (d, J=16.8 Hz, 2H), 7.63 (s, 1H), 7.34 (d, J=9.3 Hz, 1H), 7.30-7.15 (m, 4H), 7.12-7.00 (m, 2H), 5.15 (s, 1H), 4.93 (s, 1H), 4.60-4.56 (m, 1H), 4.07-3.93 (m, 1H), 2.53-2.34 (m, 3H), 2.12 (s, 1H), 1.97-1.86 (m, 1H), 1.51 (s, 1H), 1.33 (d, J=32.6 Hz, 1H), 1.01-0.91 (m, 2H), 0.78-0.63 (m, 2H).

### Example 284

### Synthesis of (1S,2S)—N-(4-(((8-(azetidin-3-yl)-6-cyclopropylimidazo[1,2-a]pyridin-2-yl)methyl)amino)pyridin-2-yl)-2-(3-chlorophenyl)cyclopropane-1-carboxamide (I-284)

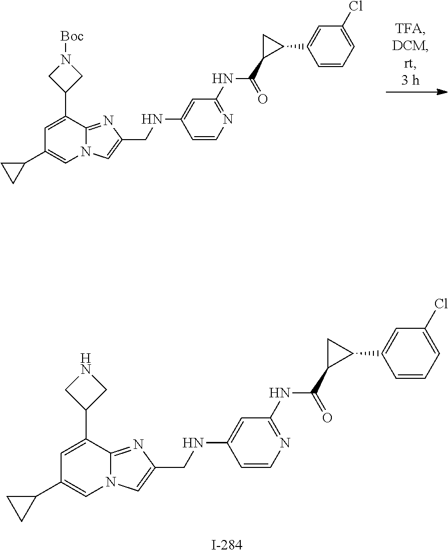 [SMILES: CC(C)(C)OC(=O)N1CC(c2cc(C3CC3)cn3cc(CNc4ccnc(NC(=O)[C@H]5C[C@@H]5c5cccc(Cl)c5)c4)nc23)C1 ; "TFA,DCM,rt,3 h" ; O=C(Nc1cc(NCc2cn3cc(C4CC4)cc(C4CNC4)c3n2)ccn1)[C@H]1C[C@@H]1c1cccc(Cl)c1]

To a solution of tert-butyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)azetidine-1-carboxylate (30 mg, 0.049 mmol) in DCM (10.0 mL) was added TFA (1 mL) at 0° C. After stirring at room temperature for 3 h, the mixture was concentrated in vacuo to give the crude, which was neutralized with a solution of ammonia in methanol, then concentrated in vacuo and purified by Pre-TLC (DCM/MeOH=10/1) to give the product (1S,2S)—N-(4-(((8-(azetidin-3-yl)-6-cyclopropylimidazo[1,2-a]pyridin-2-yl)methyl)amino)pyridin-2-yl)-2-(3-chlorophenyl)cyclopropane-1-carboxamide (6 mg, 24%) as a white solid. ESI-MS [M+H]+: 513.2. ¹H NMR (400 MHz, DMSO) δ 10.31 (s, 1H), 8.21 (s, 1H), 7.76 (d, J=5.6 Hz, 1H), 7.63 (s, 1H), 7.44 (s, 1H), 7.35-7.20 (m, 3H), 7.18-7.06 (m, 2H), 6.96 (s, 1H), 6.32 (d, J=5.5 Hz, 1H), 4.36 (d, J=5.1 Hz, 2H), 4.32-4.08 (m, 2H), 3.93 (d, J=8.1 Hz, 3H), 2.36-2.32 (m, 2H), 1.94-1.91 (m, 1H), 1.45-1.42 (m, 1H), 1.37-1.32 (m, 1H), 0.93-0.90 (m, 2H), 0.71-0.38 (m, 2H).

### Example 285

### Synthesis of (1S,2S)-2-(3-chlorophenyl)-N-(6-(((6-cyclopropyl-8-(4-methylpiperazin-1-yl)imidazo[1,2-a]pyridin-2-yl)methyl)amino)-2-hydroxypyrimidin-4-yl)cyclopropane-1-carboxamide (I-285)

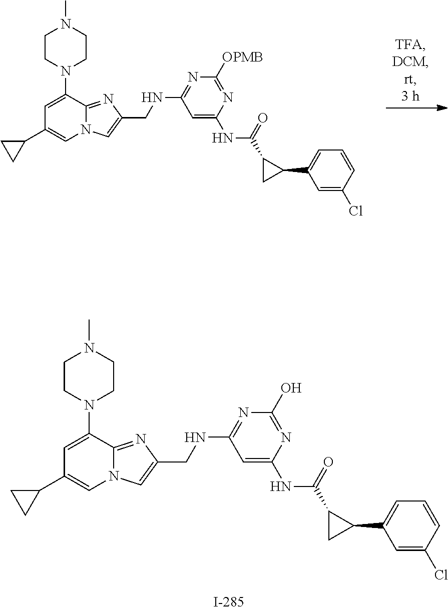

The title compound was prepared in a similar manner to (1S,2S)—N-(4-(((8-(azetidin-3-yl)-6-cyclopropylimidazo[1,2-a]pyridin-2-yl)methyl)amino)pyridin-2-yl)-2-(3-chlorophenyl)cyclopropane-1-carboxamide. ESI-MS [M+H]+: 573.2, ¹H NMR (400 MHz, DMSO) δ 10.89 (s, 1H), 10.63-10.18 (m, 1H), 8.10 (s, 1H), 7.89 (s, 1H), 7.58 (s, 1H), 7.33-7.25 (m, 3H), 7.17-7.15 (m, 1H), 6.16 (s, 1H), 5.77 (s, 1H), 4.48 (s, 2H), 3.48 (s, 4H), ), 2.50-2.49 (m, 4H), 2.46-2.44 (m, 2H), 2.24 (s, 3H), 1.89-1.82 (m, 1H), 1.54-1.43 (m, 2H), 0.89-0.84 (m, 2H), 0.67-0.64 (m, 2H).

### Example 286

### Synthesis of rac-(1S*,2S*)-2-(2-amino-6-methoxypyridin-4-yl)-N-(4-(((6-cyclopropyl imidazo[1,2-a]pyridin-2-yl)methyl)amino)pyridin-2-yl)cyclopropane-1-carboxamide (I-286)

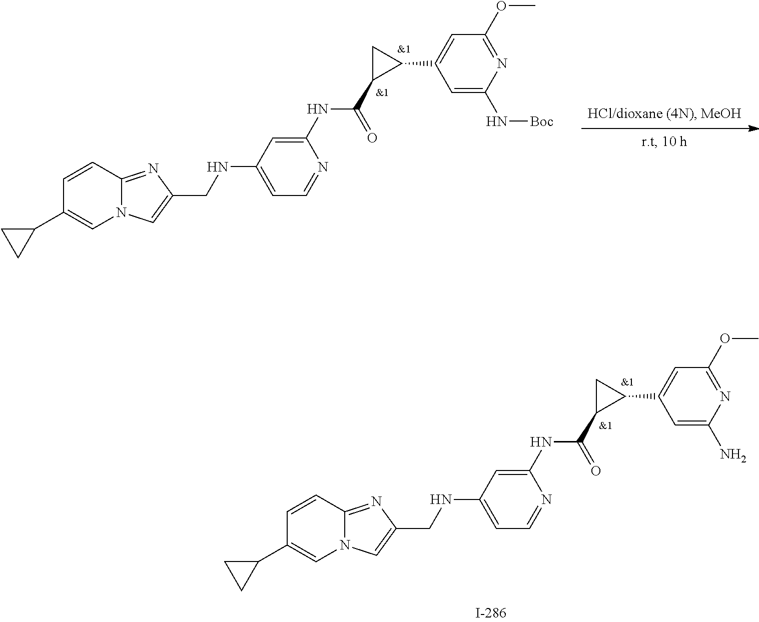 [SMILES: COc1cc([C@H]2C[C@@H]2C(=O)Nc2cc(NCc3cn4cc(C5CC5)ccc4n3)ccn2)cc(NC(=O)OC(C)(C)C)n1 ; "HCl/dioxane (4N), MeOH" ; "r.t, 10 h" ; COc1cc([C@H]2C[C@@H]2C(=O)Nc2cc(NCc3cn4cc(C5CC5)ccc4n3)ccn2)cc(N)n1]

To a solution of rac-tert-butyl (4-((1S*,2S*)-2-((4-(((6-cyclopropylimidazo[1,2-a]pyridin-2-yl)methyl)amino)pyridin-2-yl)carbamoyl)cyclopropyl)-6-methoxypyridin-2-yl)carbamate (85 mg, 0.149 mmol) in MeOH (2 mL) was added HCl (4 M in dioxane, 3 mL). After stirring at room temperature for 10 h, the reaction mixture was diluted in water (20 mL), basified to pH=9˜10 with saturated aq·NaHCO₃ (10 mL), and extracted with DCM (2×30 ml). The combined organics were washed with brine (60 mL), dried over anhydrous Na₂SO₄, and concentrated and dried in vacuo to give rac-(1S*,2S*)-2-(2-amino-6-methoxypyridin-4-yl)-N-(4-(((6-cyclopropylimidazo[1,2-a]pyridin-2-yl)methyl)amino)pyridin-2-yl)cyclopropane-1-carboxamide (33 mg, 47%) as a white solid. ESI-MS [M+H]+: 470.1, ¹H NMR (400 MHz, MeOD) δ 8.13 (s, 1H), 7.75 (d, J=6.0 Hz, 1H), 7.62 (s, 1H), 7.40-7.31 (m, 2H), 7.06 (dd, J=9.4, 1.7 Hz, 1H), 6.38 (dd, J=6.0, 2.2 Hz, 1H), 5.92 (d, J=0.9 Hz, 1H), 5.79 (d, J=1.0 Hz, 1H), 4.48 (s, 2H), 3.77 (s, 3H), 2.31-2.25 (m, 1H), 2.20-2.15 (m, 1H), 1.96-1.88 (m, 1H), 1.57-1.52 (m, 1H), 1.36-1.31 (m, 1H), 0.98-0.93 (m, 2H), 0.73-0.66 (m, 2H).

### Example 287

### Synthesis of rac-(1S*,2S*)-2-(5-chloro-2-(1H-tetrazol-1-yl)phenyl)-N-(4-(((6-cyclopropyl imidazo[1,2-a]pyridin-2-yl)methyl)amino)pyridin-2-yl)cyclopropane-1-carboxamide (I-287)

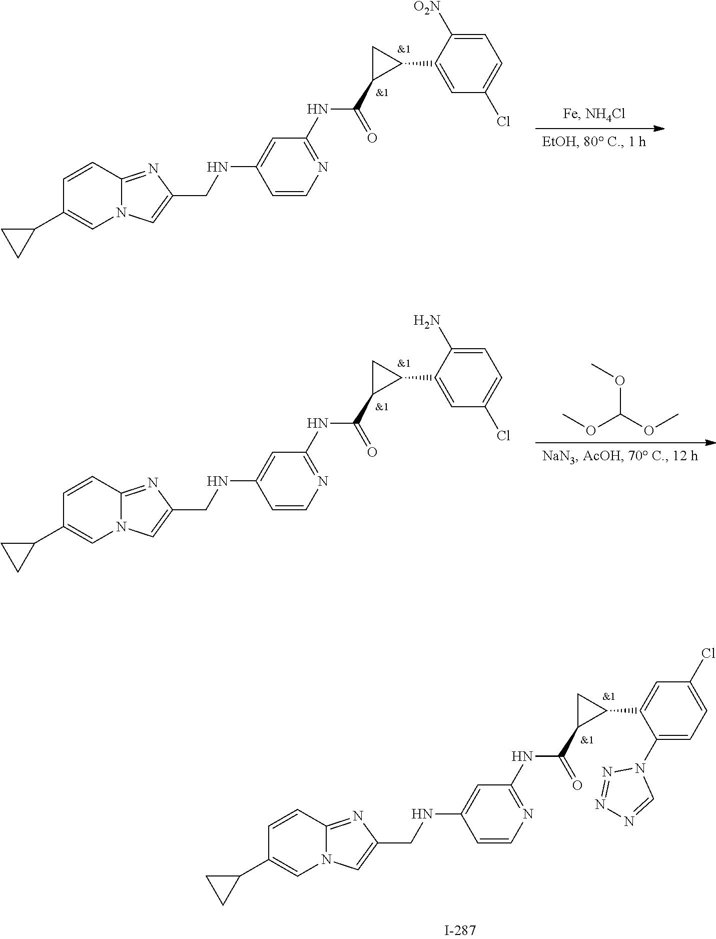

Synthesis of rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide. To a solution of rac-(1S*,2S*)-2-(5-chloro-2-nitrophenyl)-N-(4-(((6-cyclopropylimidazo[1,2-a]pyridin-2-yl)methyl)amino)pyridin-2-yl)cyclopropane-1-carboxamide (90 mg, 0.18 mmol) in EtOH (4 mL) were added Fe (50 mg, 0.90 mmol), NH₄Cl (77 mg, 1.43 mmol), and water (1 mL). After stirring at 80° C. for 1 h, the resulting mixture was cooled to room temperature and filtered, and the residue was washed with MeOH/DCM (2/1, 5×6 mL). The organic layers were concentrated in vacuo and purified by Pre-TLC (eluent: DCM/MeOH=15/1) to give rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide (60 mg, 71%) as a pale yellow solid. ESI-MS [M+H]+: 473.2.

Synthesis of rac-(1S*,2S*)-2-(5-chloro-2-(1H-tetrazol-1-yl)phenyl)-N-(4-(((6-cyclopropylimidazo[1,2-a]pyridin-2-yl)methyl)amino)pyridin-2-yl)cyclopropane-1-carboxamide (I-287). To a solution of rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide (50 mg, 0.11 mmol) in trimethoxymethane (2 mL) was added NaN₃ (21 mg, 0.32 mmol) at 0° C. After stirring at 0° C. for 40 min, AcOH (4 mL) was added and the resulting mixture was stirred at 70° C. for 12 h. The reaction mixture was cooled to room temperature, then quenched by adding water (20 mL) and extracted with EtOAc (4×20 mL). The organic layers were concentrated in vacuo and purified by preparative TLC (eluent: MeOH/DCM=15/1) to give rac-(1S*,2S*)-2-(5-chloro-2-(1H-tetrazol-1-yl)phenyl)-N-(4-(((6-cyclopropylimidazo[1,2-a]pyridin-2-yl)methyl)amino)pyridin-2-yl)cyclopropane-1-carboxamide (5.7 mg, 10%) as a white solid. ESI-MS [M+H]+: 526.1, ¹H NMR (400 MHz, DMSO) δ 10.26 (s, 1H), 9.84 (s, 1H), 8.31 (s, 1H), 7.76 (d, J=5.8 Hz, 1H), 7.63-7.59 (m, 3H), 7.54 (s, 1H), 7.39-7.36 (m, 2H), 7.11 (t, J=5.3 Hz, 1H), 6.96 (dd, J=9.3, 1.4 Hz, 1H), 6.31 (dd, J=5.7, 1.7 Hz, 1H), 4.33 (d, J=5.5 Hz, 2H), 2.36-2.28 (m, 1H), 2.05-2.00 (m, 1H), 1.94-1.87 (m, 1H), 1.33-1.26 (m, 2H), 0.92-0.88 (m, 2H), 0.68-0.64 (m, 2H).

### Example 288

### Synthesis of (1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (I-288)

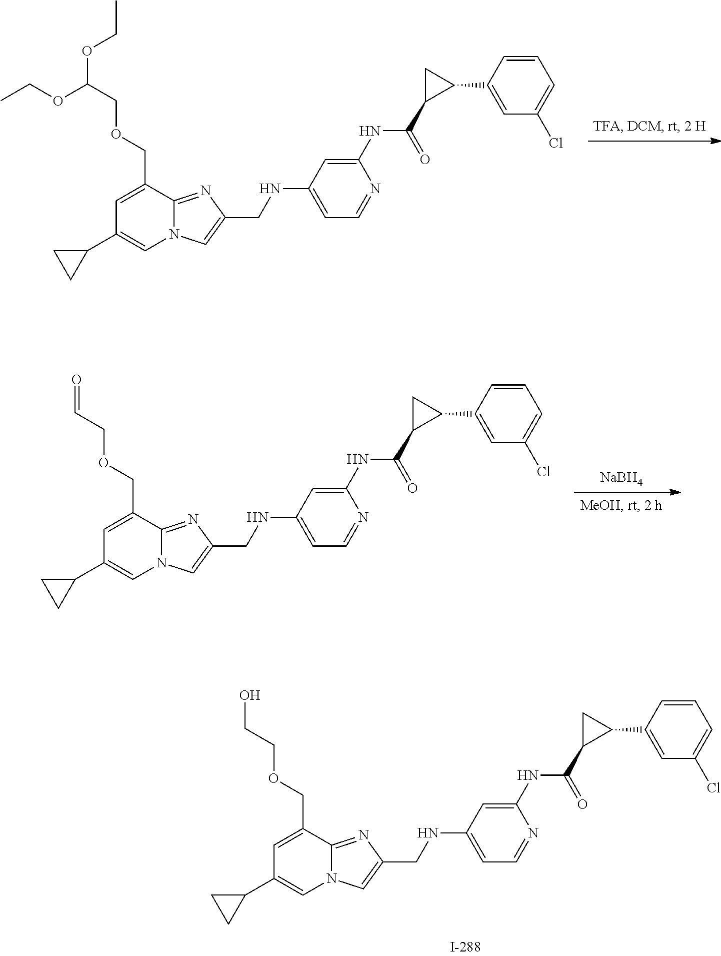 [SMILES: CCOC(COCc1cc(C2CC2)cn2cc(CNc3ccnc(NC(=O)[C@H]4C[C@@H]4c4cccc(Cl)c4)c3)nc12)OCC ; "TFA, DCM, rt, 2 H" ; O=CCOCc1cc(C2CC2)cn2cc(CNc3ccnc(NC(=O)[C@H]4C[C@@H]4c4cccc(Cl)c4)c3)nc12 ; "MeOH, rt, 2 h" ; "NaBH4" ; O=C(Nc1cc(NCc2cn3cc(C4CC4)cc(COCCO)c3n2)ccn1)[C@H]1C[C@@H]1c1cccc(Cl)c1]

Synthesis of (1S,2S)-2-(3-chlorophenyl)-N-(4-(((6-cyclopropyl-8-((2-oxoethoxy)methyl)imidazo[1,2-a]pyridin-2-yl)methyl)amino)pyridin-2-yl)cyclopropane-1-carboxamide. A mixture of (1S,2S)-2-(3-chlorophenyl)-N-(4-(((6-cyclopropyl-8-((2,2-diethoxyethoxy)methyl)-imidazo[1,2-a]pyridin-2-yl)methyl)amino)pyridin-2-yl)cyclopropane-1-carboxamide (100 mg, 0.16 mmol), TFA (0.4 ml), and DCM (4 mL) was stirred at room temperature for 2 h. Water (20 mL) was added to the mixture, which was then extracted with DCM (3×20 mL). The combined organic layers were washed with brine, dried over Na₂SO₄, filtered, and concentrated in vacuo to give (1S,2S)-2-(3-chlorophenyl)-N-(4-(((6-cyclopropyl-8-((2-oxoethoxy)methyl)imidazo[1,2-a]pyridin-2-yl)methyl)amino)pyridin-2-yl)cyclopropane-1-carboxamide (100 mg, crude) as a yellow oil. ESI-MS [M+H]+: 530.2

Synthesis of (1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (I-288). A mixture of (1S,2S)-2-(3-chlorophenyl)-N-(4-(((6-cyclopropyl-8-((2-oxoethoxy)methyl)imidazo[1,2-a]pyridin-2-yl)methyl)amino)pyridin-2-yl)cyclopropane-1-carboxamide (100 mg, crude), NaBH₄ (21.3 mg, 0.57 mmol), and MeOH (5 mL) was stirred at room temperature for 2 h. Water (20 mL) was added to the mixture, which was then extracted with EtOAc (3×20 mL). The combined organic layers were washed with brine, dried over Na₂SO₄, filtered, and concentrated in vacuo to give the crude, which was purified by silica gel chromatography (DCM/MeOH=20/1) to give (1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (45 mg, 35%) as a white solid. ESI-MS [M+H]+: 532.2, ¹H NMR (400 MHz, DMSO) δ 10.33 (s, 1H), 8.24 (s, 1H), 7.75 (d, J=5.8 Hz, 1H), 7.64 (s, 1H), 7.43 (s, 1H), 7.35-7.29 (m, 1H), 7.27-7.21 (m, 2H), 7.14 (d, J=7.6 Hz, 2H), 7.02 (s, 1H), 6.33-6.31 (m, 1H), 4.91 (s, 1H), 4.78 (s, 2H), 4.36 (d, J=5.6 Hz, 2H), 3.59 (s, 4H), 2.50-2.33 (m, 2H), 1.96-1.88 (m, 1H), 1.48-1.41 (m, 1H), 1.38-1.31 (m, 1H), 0.95-0.86 (m, 2H), 0.67-064 (m, 2H).

### Example 289

### Synthesis of rac-(1S*,2S*)-2-(5-chloro-2-cyanophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide (I-289)

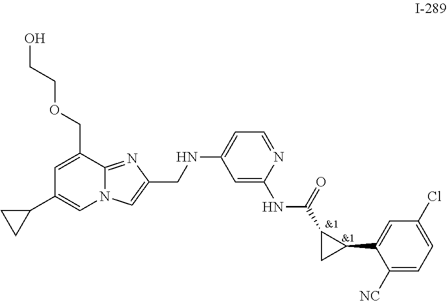 [SMILES: N#Cc1ccc(Cl)cc1[C@H]1C[C@@H]1C(=O)Nc1cc(NCc2cn3cc(C4CC4)cc(COCCO)c3n2)ccn1]

The title compound was prepared in a similar manner to (1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide. ESI-MS [M+H]+: 557.1. ¹H NMR (400 MHz, DMSO) δ 10.39 (s, 1H), 8.24 (s, 1H), 7.86 (d, J=8.3 Hz, 1H), 7.77 (d, J=5.8 Hz, 1H), 7.65 (s, 1H), 7.52 (dd, J=8.3, 2.0 Hz, 1H), 7.46 (s, 1H), 7.39 (d, J=1.8 Hz, 1H), 7.17 (t, J=5.1 Hz, 1H), 7.01 (s, 1H), 6.31 (dd, J=5.8, 1.9 Hz, 1H), 4.95 (s, 1H), 4.78 (s, 2H), 4.36 (d, J=5.7 Hz, 2H), 3.59 (s, 4H), 2.60-2.53 (m, 2H), 1.95-1.88 (m, 1H), 1.56-1.45 (m, 2H), 0.93-0.83 (m, 2H), 0.77-0.64 (m, 2H).

### Example 290

### Synthesis of 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoic acid (I-290)

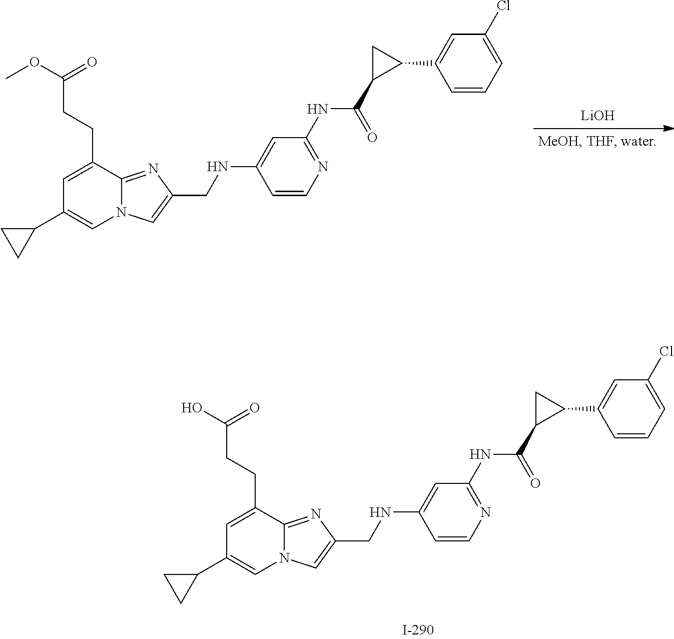 [SMILES: COC(=O)CCc1cc(C2CC2)cn2cc(CNc3ccnc(NC(=O)[C@H]4C[C@@H]4c4cccc(Cl)c4)c3)nc12 ; "LiOH" ; "MeOH, THF, water." ; O=C(O)CCc1cc(C2CC2)cn2cc(CNc3ccnc(NC(=O)[C@H]4C[C@@H]4c4cccc(Cl)c4)c3)nc12]

A mixture of methyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate (40 mg, 0.074 mmol) and LiOH·H₂O (15.5 mg, 0.37 mmol) in MeOH/THF/water (1.0 mL/1.0 mL/1.0 mL) was stirred at room temperature for 1 h. The mixture was concentrated and purified by preparative HPLC (FA) to give the desired product 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoic acid (21 mg, 54%) as a white solid. ESI-MS [M+H]+: 530.2, ¹H NMR (400 MHz, DMSO) δ 12.94 (s, 1H), 10.31 (s, 1H), 8.19 (s, 1H), 7.61 (s, 2H), 7.27-7.24 (m, 6H), 6.81 (s, 1H), 6.38 (s, 1H), 4.33 (s, 2H), 3.07 (s, 2H), 2.85-2.61 (m, 2H), 2.33 (s, 2H), 1.87-1.86 (m, 1H), 1.45-1.35 (m, 2H), 0.90-0.86 (m, 2H), 0.65-0.64 (m, 2H).

### Example 291

### Synthesis of (1S,2S)-2-(3-chlorophenyl)-N-(4-(((6-cyclopropyl-8-(3-hydroxypropyl)imidazo[1,2-a]pyridin-2-yl)methyl)amino)pyridin-2-yl)cyclopropane-1-carboxamide (I-291)

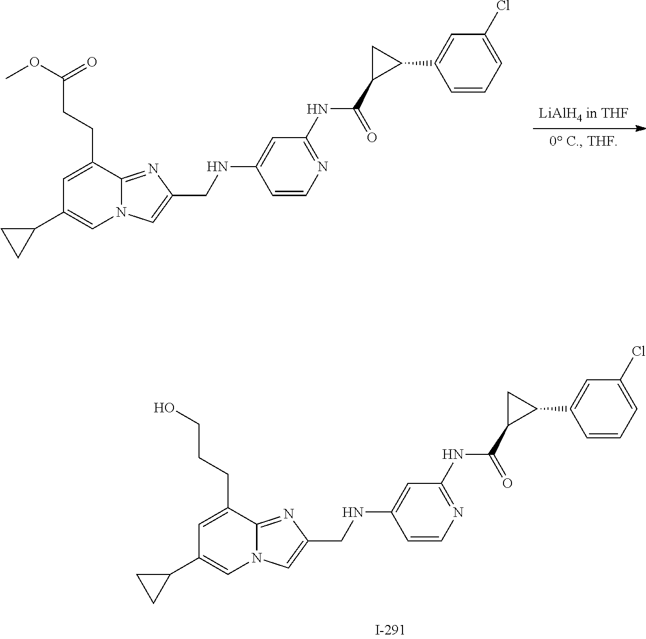 [SMILES: COC(=O)CCc1cc(C2CC2)cn2cc(CNc3ccnc(NC(=O)[C@H]4C[C@@H]4c4cccc(Cl)c4)c3)nc12 ; "0° C., THF." ; "LiAlH4 in THF" ; O=C(Nc1cc(NCc2cn3cc(C4CC4)cc(CCCO)c3n2)ccn1)[C@H]1C[C@@H]1c1cccc(Cl)c1]

To a mixture of methyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate (70 mg, 0.129 mmol) in THF (5.0 mL) was added LiAlH₄ (0.19 ml, 1M, 0.19 mmol). After stirring at 0° C. under N₂ for 1 h, the mixture was quenched with water (1.0 mL), concentrated in vacuo, and purified by preparative HPLC (FA) to give the desired product ((1S,2S)-2-(3-chlorophenyl)-N-(4-(((6-cyclopropyl-8-(3-hydroxypropyl)imidazo[1,2-a]pyridin-2-yl)methyl)amino)pyridin-2-yl)cyclopropane-1-carboxamide (26 mg, 39%) as a white solid. ESI-MS [M+H]+: 516.2, ¹H NMR (400 MHz, DMSO) δ 10.33 (s, 1H), 8.15-8.13 (m, 1H), 7.81-7.12 (m, 9H), 6.55-6.50 (m, 1H), 4.35 (s, 2H), 2.86-2.84 (m, 2H), 2.36-2.30 (m, 4H), 1.86-1.83 (m, 3H), 1.39-1.37 (m, 2H), 0.89-0.86 (m, 2H), 0.65-0.62 (m, 2H).

### Example 292

### Synthesis of (1S,2S)-2-(3-chlorophenyl)-N-(4-(((6-cyclopropyl-8-(2-hydroxyethyl)imidazo[1,2-a]pyridin-2-yl)methyl)amino)pyridin-2-yl)cyclopropane-1-carboxamide (I-292)

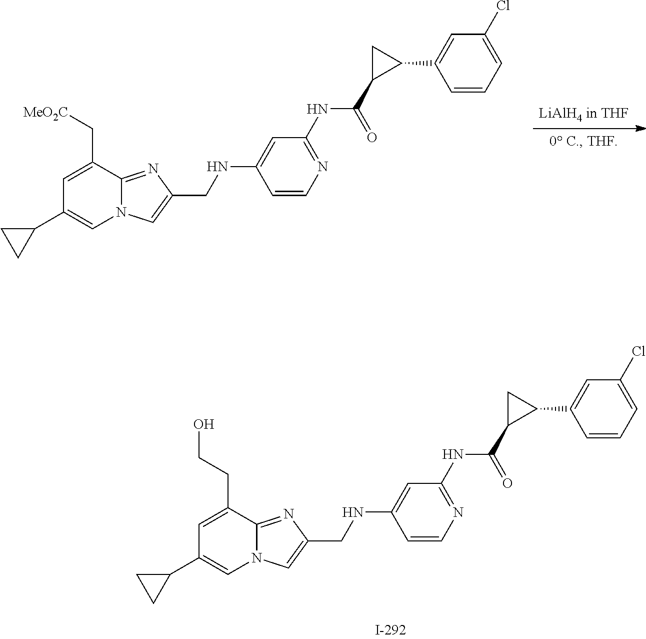 [SMILES: COC(=O)Cc1cc(C2CC2)cn2cc(CNc3ccnc(NC(=O)[C@H]4C[C@@H]4c4cccc(Cl)c4)c3)nc12 ; "0° C., THF." ; "LiAlH4 in THF" ; O=C(Nc1cc(NCc2cn3cc(C4CC4)cc(CCO)c3n2)ccn1)[C@H]1C[C@@H]1c1cccc(Cl)c1]

The title compound was prepared in a similar manner to (1S,2S)-2-(3-chlorophenyl)-N-(4-(((6-cyclopropyl-8-(3-hydroxypropyl)imidazo[1,2-a]pyridin-2-yl)methyl)amino)pyridin-2-yl)cyclopropane-1-carboxamide. ESI-MS [M+H]+: 502.1. ¹H NMR (400 MHz, CDCl₃) δ 7.75-7.74 (m, 2H), 7.58 (s, 1H), 7.49 (s, 1H), 7.21-7.19 (m, 2H), 7.09 (s, 1H), 7.01 (d, J=6.7 Hz, 1H), 6.76 (s, 1H), 6.31-6.28 (m, 1H), 5.27 (s, 1H), 4.52 (d, J=5.6 Hz, 2H), 3.97-3.93 (m, 2H), 3.16-3.14 (m, 2H), 2.57 (s, 1H), 1.88-1.84 (m, 2H), 1.72-1.70 (m, 1H), 1.37-1.31 (m, 2H), 0.95 (d, J=8.2 Hz, 2H), 0.66 (d, J=5.1 Hz, 2H).

### Example 293

### Synthesis of (1S,2S)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (I-293)

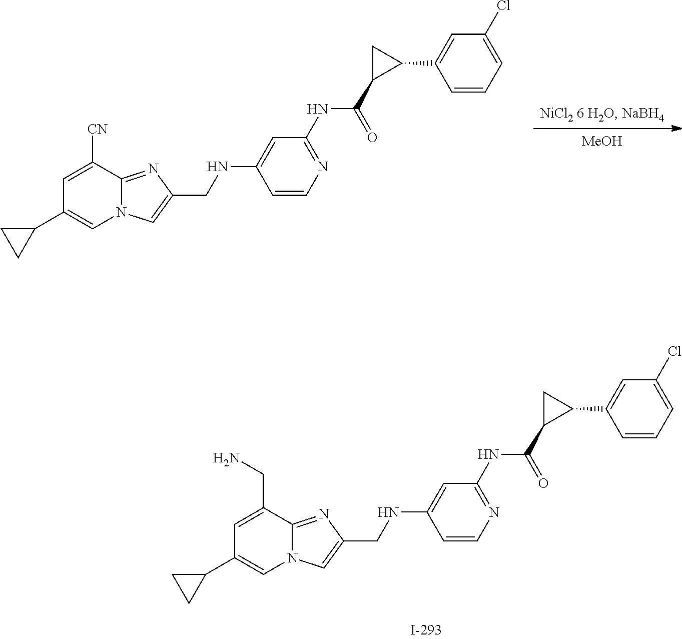 [SMILES: "MeOH" ; N#Cc1cc(C2CC2)cn2cc(CNc3ccnc(NC(=O)[C@H]4C[C@@H]4c4cccc(Cl)c4)c3)nc12 ; "NiCl2 6 H2O, NaBH4" ; NCc1cc(C2CC2)cn2cc(CNc3ccnc(NC(=O)[C@H]4C[C@@H]4c4cccc(Cl)c4)c3)nc12]

To a mixture of (1S,2S)-2-(3-chlorophenyl)-N-(4-(((8-cyano-6-cyclopropylimidazo[1,2-a]pyridin-2-yl)methyl)amino)pyridin-2-yl)cyclopropane-1-carboxamide (70 mg, 0.145 mmol) in MeOH (5.0 mL) was added NiCl₂·6H₂O (34.6 mg, 0.145 mmol) at 0° C., and then NaBH₄ (15.7 mg, 0.435 mmol) was added. After stirring at 0° C. under N₂ for 1 h, the mixture was quenched with water (1.0 mL), concentrated in vacuo, and purified by preparative HPLC (FA) to give the desired product (1S,2S)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide (6.1 mg, 8.7%) as a white solid. ESI-MS [M+H]+: 487.2. ¹H NMR (400 MHz, DMSO) δ 10.33 (s, 1H), 8.65-8.05 (m, 4H), 7.82-7.59 (m, 2H), 7.45 (s, 1H), 7.37-7.20 (m, 3H), 7.16-7.14 (m, 2H), 7.03 (s, 1H), 6.32 (s, 1H), 4.38 (s, 2H), 4.14 (s, 2H), 2.39-2.36 (m, 2H), 1.97-1.76 (m, 1H), 1.50-1.40 (m, 1H), 1.38-1.30 (m, 1H), 0.93-0.92 (m, 2H), 0.69-0.67 (m, 2H).

### Example 294

### Synthesis of rac-(1S*,2S*)-2-(3-chlorophenyl)-N-(4-(((6-cyclopropyl-8-(3-fluorooxetan-3-yl)imidazo[1,2-a]pyridin-2-yl)methyl)amino)pyridin-2-yl)cyclopropane-1-carboxamide (I-294)

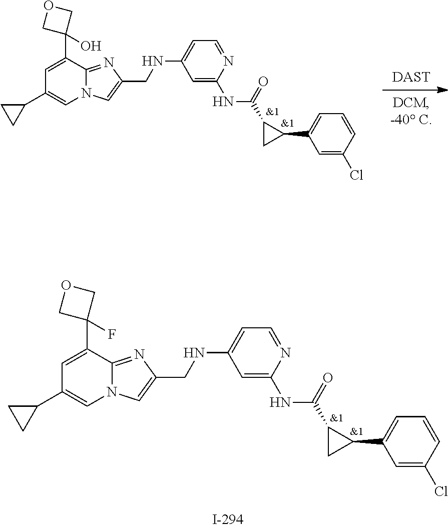 [SMILES: O=C(Nc1cc(NCc2cn3cc(C4CC4)cc(C4(O)COC4)c3n2)ccn1)[C@H]1C[C@@H]1c1cccc(Cl)c1 ; "DAST" ; "DCM,-40° C." ; O=C(Nc1cc(NCc2cn3cc(C4CC4)cc(C4(F)COC4)c3n2)ccn1)[C@H]1C[C@@H]1c1cccc(Cl)c1]

To a mixture of rac-(1S*,2S*)-2-(3-chlorophenyl)-N-(4-(((6-cyclopropyl-8-(3-hydroxyoxetan-3-yl)imidazo[1,2-a]pyridin-2-yl)methyl)amino)pyridin-2-yl)cyclopropane-1-carboxamide (20 mg, 0.038 mmol) in DCM (5 ml) was added DAST (30 mg, 0.19 mmol). The mixture was stirred at −40° C. for 3 h under N₂. Water (10 mL) was added to the reaction, which was then extracted with EtOAc (3×10 mL). The combined organic layers were washed with brine, dried over Na₂SO₄, and concentrated in vacuo to give the crude, which was purified with silica gel chromatography (DCM/MeOH=10/1) to give the rac-(1S*,2S*)-2-(3-chlorophenyl)-N-(4-(((6-cyclopropyl-8-(3-fluorooxetan-3-yl)imidazo[1,2-a]pyridin-2-yl)methyl)amino)pyridin-2-yl)cyclopropane-1-carboxamide (7 mg, 35%) as a white solid. ESI-MS [M+H]+: 532.2, ¹H NMR (400 MHz, DMSO) δ 10.31 (s, 1H), 8.40 (s, 1H), 7.76 (d, J=5.7 Hz, 1H), 7.69 (s, 1H), 7.45 (s, 1H), 7.35-7.20 (m, 3H), 7.18-7.11 (m, 3H), 6.35-6.31 (m, 1H), 5.39 (d, J=8.6 Hz, 1H), 5.32 (d, J=8.6 Hz, 1H), 5.01 (d, J=8.6 Hz, 1H), 4.95 (d, J=8.6 Hz, 1H), 4.38 (d, J=5.8 Hz, 2H), 2.38-2.32 (m, 2H), 2.00-1.89 (m, 1H), 1.48-1.42 (m, 1H), 1.36-1.32 (m, 1H), 0.97-0.86 (m, 2H), 0.77-0.63 (m, 2H).

### Example 295

### Synthesis of (1S,2S)-2-(3-chlorophenyl)-N-(4-(((6-cyclopropylimidazo[1,2-a]pyridin-2-yl)methyl)amino)-6-hydroxy-1,3,5-triazin-2-yl)cyclopropane-1-carboxamide (I-295)

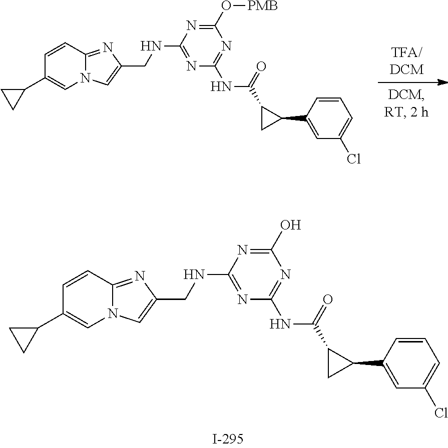

A mixture of (1S,2S)-2-(3-chlorophenyl)-N-(4-(((6-cyclopropylimidazo[1,2-a]pyridin-2-yl)methyl)amino)-6-((4-methoxybenzyl)oxy)-1,3,5-triazin-2-yl)cyclopropane-1-carboxamide (45 mg, 0.1 mmol) in TFA/DCM (1 mL/3 mL) was stirred at room temperature for 2 h. The reaction mixture was neutralized with saturated NaHCO₃ solution (20 mL) to pH˜8 and extracted with DCM (5×30 mL). The combined organic layers were washed with brine (30 mL), dried over Na₂SO₄, and concentrated in vacuo. The residue was purified by preparative TLC (eluent: MeOH/DCM=1/8) to afford (1S,2S)-2-(3-chlorophenyl)-N-(4-(((6-cyclopropylimidazo[1,2-a]pyridin-2-yl)methyl)amino)-6-hydroxy-1,3,5-triazin-2-yl)cyclopropane-1-carboxamide (10 mg, 26% yield) as a white solid. ESI-MS [M+H]+: 476.2. ¹H NMR (400 MHz, CDCl₃) δ 14.12 (s, 1H), 11.45 (s, 1H), 8.64 (s, 1H), 7.78 (s, 1H), 7.61 (s, 1H), 7.10-6.98 (m, 2H), 6.74 (d, J=7.4 Hz, 1H), 6.67-6.60 (m, 2H), 6.36 (d, J=8.9 Hz, 1H), 5.15 (s, 1H), 4.37 (d, J=13.9 Hz, 1H), 2.60 (s, 1H), 2.20 (s, 1H), 2.02 (s, 1H), 1.81-1.75 (m, 2H), 0.98-0.90 (m, 2H), 0.62-0.55 (m, 2H).

### Example 296

### Synthesis of (1S,2S)-2-(3-chlorophenyl)-N-(6-(((6-cyclopropylimidazo[1,2-a]pyridin-2-yl)methyl)amino)-2-oxo-2,3-dihydropyrimidin-4-yl)cyclopropane-1-carboxamide (I-296)

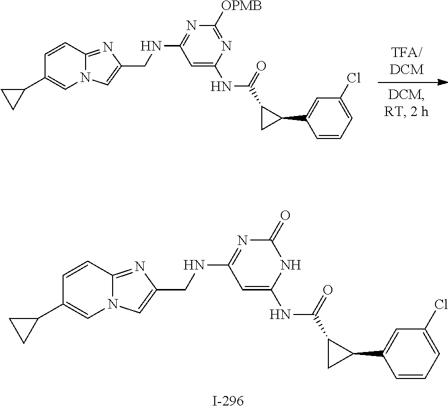

The title compound was prepared in a similar manner to (1S,2S)-2-(3-chlorophenyl)-N-(4-(((6-cyclopropylimidazo[1,2-a]pyridin-2-yl)methyl)amino)-6-hydroxy-1,3,5-triazin-2-yl)cyclopropane-1-carboxamide. ESI-MS [M+H]+: 475.1, ¹H NMR (400 MHz, DMSO) δ=10.91 (s, 1H), 10.45 (s, 1H), 8.33 (s, 1H), 8.00 (s, 1H), 7.67 (s, 1H), 7.44-7.37 (m, 1H), 7.35-7.22 (m, 3H), 7.20-7.10 (m, 1H), 6.98 (d, J=9.2, 1H), 5.76 (s, 1H), 4.50 (s, 2H), 2.46-2.35 (m, 1H), 2.25-2.06 (m, 1H), 1.97-1.87 (m, 1H), 1.56-1.38 (m, 2H), 1.00-0.80 (m, 2H), 0.74-0.47 (m, 2H).

### Example 297

### Synthesis of (1S,2S)—N-(6-((2R,4S)-4-amino-2-(6-cyclopropylimidazo[1,2-a]pyridin-2-yl)pyrrolidin-1-yl)pyrimidin-4-yl)-2-(3-chlorophenyl)cyclopropane-1-carboxamide (I-297)

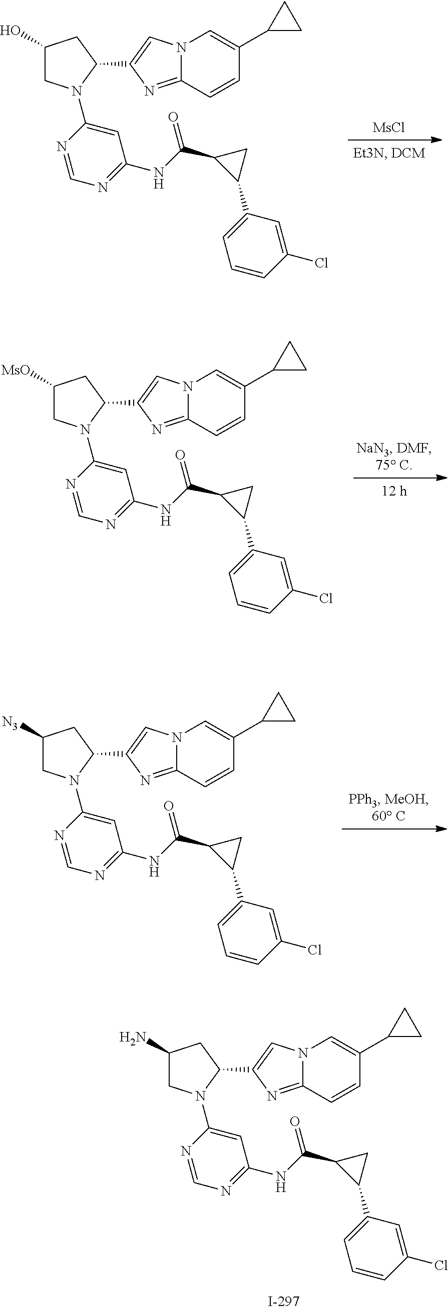

Synthesis of (3R,5R)-1-(6-((1S,2S)-2-(3-chlorophenyl)cyclopropane-1-carboxamido)pyrimidin-4-yl)-5-(6-cyclopropylimidazo[1,2-a]pyridin-2-yl)pyrrolidin-3-yl methanesulfonate. To a solution of (1S,2S)-2-(3-chlorophenyl)-N-(6-((2R,4R)-2-(6-cyclopropylimidazo[1,2-a]pyridin-2-yl)-4-hydroxypyrrolidin-1-yl)pyrimidin-4-yl)cyclopropane-1-carboxamide (60 mg, 0.12 mmol) in DCM (5 ml) was added Et₃N (36 mg, 0.36 mmol) and MsCl (41 mg, 0.36 mmol) at 0° C. After stirring at room temperature for 3 h, the mixture was quenched with water (20 mL) and extracted with DCM (3×30 mL). The organic layer was concentrated to give (3R,5R)-1-(6-((1S,2S)-2-(3-chlorophenyl)cyclopropane-1-carboxamido)pyrimidin-4-yl)-5-(6-cyclopropylimidazo[1,2-a]pyridin-2-yl)pyrrolidin-3-yl methanesulfonate (60 mg, crude) as a yellow oil. ESI-MS [M+H]⁺: 593.2.

Synthesis of (1S,2S)—N-(6-((2R,4S)-4-azido-2-(6-cyclopropylimidazo[1,2-a]pyridin-2-yl)pyrrolidin-1-yl)pyrimidin-4-yl)-2-(3-chlorophenyl)cyclopropane-1-carboxamide. A solution of (3R,5R)-1-(6-((1S,2S)-2-(3-chlorophenyl)cyclopropane-1-carboxamido)pyrimidin-4-yl)-5-(6-cyclopropylimidazo[1,2-a]pyridin-2-yl)pyrrolidin-3-yl methanesulfonate (60 mg, crude) and NaN₃ (10 mg, 0.15 mmol) in DMF (10 mL) was stirred at 75° C. for 12 h. After cooling to room temperature, water (30 mL) was added, and the mixture was extracted with EtOAc (3×30 mL). The combined organic layers were washed with brine, dried over Na₂SO₄, filtered. and concentrated in vacuo to give the crude, which was purified by preparative TLC to give (1S,2S)—N-(6-((2R,4S)-4-azido-2-(6-cyclopropylimidazo[1,2-a]pyridin-2-yl)pyrrolidin-1-yl)pyrimidin-4-yl)-2-(3-chlorophenyl)cyclopropane-1-carboxamide (40 mg, 74%) as a yellow oil. ESI-MS [M+H]⁺: 540.2.

Synthesis of (1S,2S)—N-(6-((2R,4S)-4-amino-2-(6-cyclopropylimidazo[1,2-a]pyridin-2-yl)pyrrolidin-1-yl)pyrimidin-4-yl)-2-(3-chlorophenyl)cyclopropane-1-carboxamide (I-297). A solution of (1S,2S)—N-(6-((2R,4S)-4-azido-2-(6-cyclopropylimidazo[1,2-a]pyridin-2-yl)pyrrolidin-1-yl)pyrimidin-4-yl)-2-(3-chlorophenyl)cyclopropane-1-carboxamide (40 mg, 0.07 mmol) and PPh₃ (55 mg, 0.21 mmol) in MeOH (5 mL) was stirred at 60° C. for 2 h under N₂. After cooling to room temperature, water (15 mL) was added, and the mixture was extracted with EtOAc (3×15 mL). The combined organic layers were washed with brine, dried over Na₂SO₄, filtered, and concentrated in vacuo to give the crude, which was purified by preparative TLC to give (1S,2S)—N-(6-((2R,4R)-4-amino-2-(6-cyclopropylimidazo[1,2-a]pyridin-2-yl)pyrrolidin-1-yl)pyrimidin-4-yl)-2-(3-chlorophenyl)cyclopropane-1-carboxamide (20 mg, 56%) as a yellow solid. ESI-MS [M+H]+: 514.2. ¹H NMR (400 MHz, MeOD) δ 8.19 (s, 2H), 7.79 (s, 1H), 7.44-7.33 (m, 2H), 7.28-7.12 (m, 3H), 7.09-6.98 (m, 2H), 5.36-5.15 (m, 1H), 4.15-3.72 (m, 3H), 2.96-2.77 (m, 1H), 2.46-2.42 (m, 1H), 2.28-2.21 (m, 1H), 2.16-2.12 (m, 1H), 1.96-1.88 (m, 1H), 1.64-1.58 (m, 1H), 1.43-1.34 (m, 1H), 0.98-0.91 (m, 2H), 0.73-0.68 (m, 2H).

### Example 298

### Synthesis of 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid (I-298)

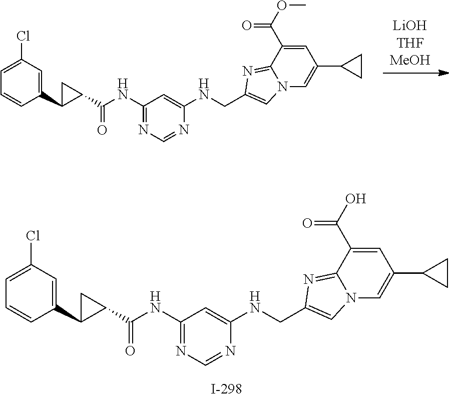 [SMILES: COC(=O)c1cc(C2CC2)cn2cc(CNc3cc(NC(=O)[C@H]4C[C@@H]4c4cccc(Cl)c4)ncn3)nc12 ; "LiOHTHFMeOH" ; O=C(O)c1cc(C2CC2)cn2cc(CNc3cc(NC(=O)[C@H]4C[C@@H]4c4cccc(Cl)c4)ncn3)nc12]

Methyl 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylate (160 mg, 0.31 mmol) was dissolved in THF (10 mL) and MeOH (0.5 mL), and lithium hydroxide was added (8.2 mg, 0.34 mmol). The resulting reaction mixture was stirred at room temperature for 16 h. The solvent was removed under reduced pressure to give the title compound as a lithium salt (160 mg). 140 mg was used without further purification, however 20 mg was purified by preparative HPLC to give the title compound (6 mg). ESI-MS (M+H)+: 503.3, ¹H NMR (400 MHz, DMSO): δ 10.64 (s, 1H), 8.58 (s, 1H), 8.23 (s, 1H), 8.02 (br s, 1H), 7.80 (s, 1H), 7.69 (s, 1H), 7.36-7.26 (m, 4H), 7.17 (ddd, J=1.4, 1.4, 7.7 Hz, 1H), 4.67 (br s, 2H), 2.41-2.40 (m, 2H), 2.05-2.05 (m, 1H), 1.49-1.48 (m, 1H), 1.43-1.42 (m, 1H), 0.98-0.98 (m, 2H), 0.74-0.74 (m, 2H).

### Example 299

### Synthesis of 2-(((6-((1S,2S)-2-(3-chlorophenyl)cyclopropane-1-carboxamido)pyrimidin-4-yl)amino)methyl)-6-cyclopropyl-N,N-dimethylimidazo(1,2-a pyridine-8-carboxamide (I-299)

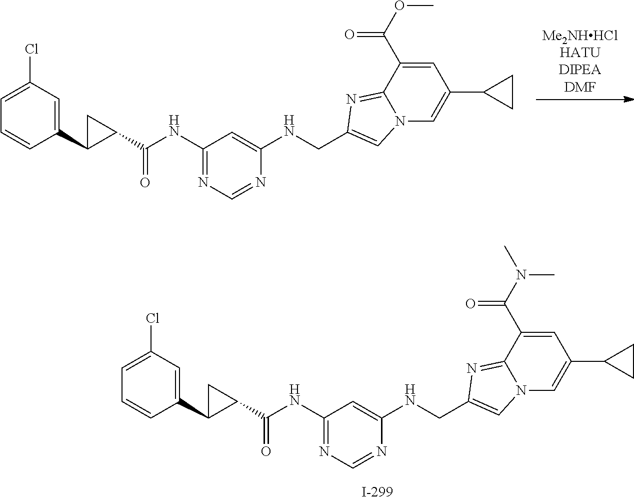 [SMILES: COC(=O)c1cc(C2CC2)cn2cc(CNc3cc(NC(=O)[C@H]4C[C@@H]4c4cccc(Cl)c4)ncn3)nc12 ; "Me2NH·HClHATUDIPEADMF" ; CN(C)C(=O)c1cc(C2CC2)cn2cc(CNc3cc(NC(=O)[C@H]4C[C@@H]4c4cccc(Cl)c4)ncn3)nc12]

2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid (70 mg, 0.14 mmol) was dissolved in DMF (1 mL), and HATU (58 mg, 1.10 mmol) and DIPEA (0.048 mL, 0.28 mmol) were added. The resulting mixture was stirred at room temperature, and then dimethylamine hydrochloride (11 mg, 0.139 mmol) was added. The reaction mixture was stirred at room temperature for 16 h. The reaction mixture was diluted with water (20 mL) and brine solution (20 mL) and extracted with EtOAc (2×20 mL). The combined organics were washed with brine (20 mL) and passed through a hydrophobic frit. The solvent was removed in vacuo and the residue was purified by silica gel chromatography (eluting with a gradient of 0-20% (7N NH₃ in MeOH) in DCM to give the title compound (33 mg, 45%) as a formic acid salt. ESI-MS (M+H)+: 530.4, ¹H NMR (400 MHz, DMSO): δ 10.61 (s, 1H), 8.37 (d, J=1.6 Hz, 1H), 8.21 (d, J=1.0 Hz, 1H), 7.92 (br s, 1H), 7.68 (s, 1H), 7.36-7.26 (m, 4H), 7.16 (ddd, J=1.3, 1.3, 7.6 Hz, 1H), 6.95 (d, J=1.7 Hz, 1H), 4.58 (s, 2H), 3.05 (s, 3H), 2.81 (s, 3H), 2.44-2.38 (m, 2H), 1.98-1.91 (m, 1H), 1.51-1.46 (m, 1H), 1.44-1.39 (m, 1H), 0.96-0.90 (m, 2H), 0.72-0.67 (m, 2H).

Using a procedure similar to that for 2-(((6-((1S,2S)-2-(3-chlorophenyl)cyclopropane-1-carboxamido)pyrimidin-4-yl)amino)methyl)-6-cyclopropyl-N,N-dimethylimidazo[1,2-a]pyridine-8-carboxamide, the compounds in Table 27 were synthesized from 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid and an appropriate coupling partner.

TABLE 27

| | | Coupling Partner / |
|---|---|---|
| Example | Compound | Analytical Data |
| Example 300 | 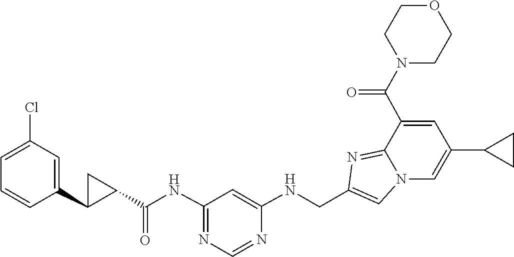 [SMILES: O=C(Nc1cc(NCc2cn3cc(C4CC4)cc(C(=O)N4CCOCC4)c3n2)ncn1)[C@H]1C[C@@H]1c1cccc(Cl)c1] | Morpholine ESI-MS (M + H)+: 572.3, ¹H NMR (400 MHz, DMSO) δ 10.59 (s, 1H), 8.37 - 8.35 (m, 1H), 8.19 (s, 1H), 7.87 (br s, 1H), 7.67 (s, 1H), 7.34 - 7.24 (m, 4H), 7.16 - 7.13 (m, 1H), 7.00 (d, J = 1.5 Hz, 1H), 4.56 (br s, 2H), 3.67 (s, 4H), 3.55 - 3.50 (m, 2H), 3.16 - 3.12 (m, 2H), 2.42 - 2.34 (m, 2H), 1.97 - 1.90 (m, 1H), 1.49 - 1.36 (m, 2H), 0.95 - 0.89 (m, 2H), 0.71 - 0.66 (m, 2H). |
| | (1S,2S)-2-(3-chlorophenyl)-N-(6-(((6- | |
| | cyclopropyl-8-(morpholine-4- | |
| | carbonyl)imidazo[1,2-a]pyridin-2- | |
| | yl)methyl)amino)pyrimidin-4- | |
| | yl)cyclopropane-1-carboxamide (I-300) | |
| Example 301 | 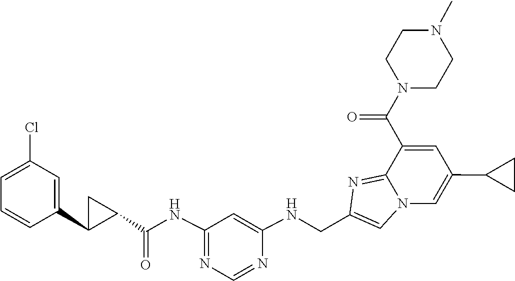 [SMILES: CN1CCN(C(=O)c2cc(C3CC3)cn3cc(CNc4cc(NC(=O)[C@H]5C[C@@H]5c5cccc(Cl)c5)ncn4)nc23)CC1] | 1-methylpiperazine ESI-MS (M + H)+: 585.3, ¹H NMR (400 MHz, DMSO) δ 10.61 (s, 1H), 8.37 (d, J = 1.4 Hz, 1H), 8.20 (d, J = 0.8 Hz, 1H), 7.90 (br s, 1H), 7.68 (s, 1H), 7.36 - 7.25 (m, 4H), 7.16 (ddd, J = 1.3, 1.3, 7.6 Hz, 1H), 6.97 (d, J = 1.6 Hz, 1H), 4.58 (br s, 2H), 3.67 (t, J = 4.5 Hz, 2H), 3.13 (t, J = 4.7 Hz, 2H), 2.41 - 2.36 (m, 4H), 2.25 (br s, 2H), 1.98 - 1.91 (m, 1H), 1.50 - 1.46 (m, 2H), 1.44 - 1.39 (m, 1H), 0.97 - 0.91 (m, 2H), 0.72 - 0.67 (m, 2H). |
| | (1S,2S)-2-(3-chlorophenyl)-N-(6-(((6- | |
| | cyclopropyl-8-(4-methylpiperazine-1- | |
| | carbonyl)imidazo[1,2-a]pyridin-2- | |
| | yl)methyl)amino)pyrimidin-4- | |
| | yl)cyclopropane-1-carboxamide (I-301) | |
| Example 302 | 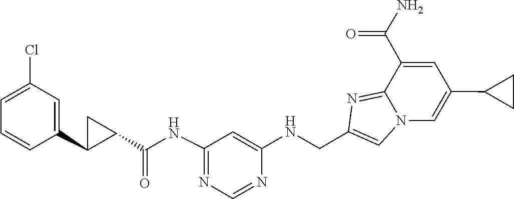 [SMILES: NC(=O)c1cc(C2CC2)cn2cc(CNc3cc(NC(=O)[C@H]4C[C@@H]4c4cccc(Cl)c4)ncn3)nc12] | Ammonium carbonate ESI-MS (M + H)+: 502.4, ¹H NMR (400 MHz, DMSO) δ 10.62 (s, 1H), 9.49 (s, 1H), 8.55 (d, J = 1.4 Hz, 1H), 8.22 (d, J = 0.7 Hz, 1H), 8.00 - 7.96 (m, 2H), 7.78 (s, 1H), 7.73 (d, J = 1.8 Hz, 1H), 7.34 - 7.26 (m, 4H), 7.17 (ddd, J = 1.3, 1.3, 7.6 Hz, 1H), 4.65 (br s, 2H), 2.45 - 2.38 (m, 2H), 2.06 - 1.99 (m, 1H), 1.51 - 1.40 (m, 2H), 1.00 - 0.95 (m, 2H), 0.73 - 0.69 (m, 2H). |
| | 2-(((6-((1S,2S)-2-(3- | |
| | chlorophenyl)cyclopropane-1- | |
| | carboxamido)pyrimidin-4- | |
| | yl)amino)methyl)-6- | |
| | cyclopropylimidazo[1,2-a]pyridine-8- | |
| | carboxamide (I-302) | |
| Example 303 | 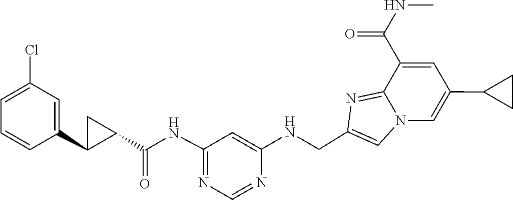 [SMILES: CNC(=O)c1cc(C2CC2)cn2cc(CNc3cc(NC(=O)[C@H]4C[C@@H]4c4cccc(Cl)c4)ncn3)nc12] | Methylamine hydrochloride ESI-MS (M + H)+: 516.3, ¹H NMR (400 MHz, DMSO) δ 10.64 (s, 1H), 9.94 (d, J = 4.5 Hz, 1H), 8.53 (d, J = 1.5 Hz, 1H), 8.22 (s, 1H), 7.96 (br s, 1H), 7.78 (s, 1H), 7.75 (d, J = 1.7 Hz, 1H), 7.36 - 7.26 (m, 4H), 7.17 (ddd, J = 1.4, 1.4, 7.5 Hz, 1H), 4.65 (br s, 2H), 2.97 (d, J = 4.8 Hz, 3H), 2.45 - 2.37 (m, 2H), 2.07 - 2.00 (m, 1H), 1.51 - 1.40 (m, 2H), 1.01 - 0.95 (m, 2H), 0.73 - 0.68 (m, 2H). |
| | 2-(((6-((1S,2S)-2-(3- | |
| | chlorophenyl)cyclopropane-1- | |
| | carboxamido)pyrimidin-4- | |
| | yl)amino)methyl)-6-cyclopropyl-N- | |
| | methylimidazo[1,2-a]pyridine-8- | |
| | carboxamide (I-303) | |

### Example 304

### Synthesis of (1S,2S)-2-(3-chlorophenyl)-N-(6-(((6-cyclopropyl-8-(piperazine-1-carbonyl)imidazo[1,2-a]pyridin-2-yl)methyl)amino)pyrimidin-4-yl)cyclopropane-1-carboxamide (I-304)

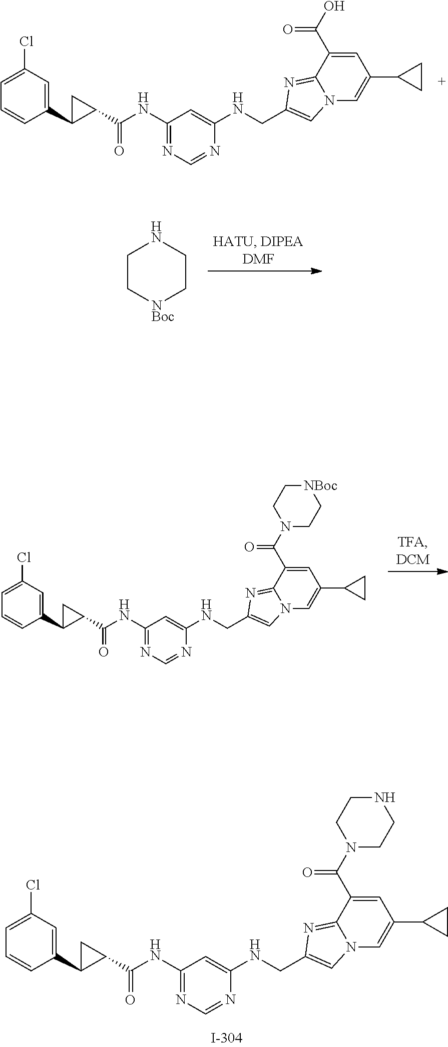

Synthesis of tert-butyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridine-8-carbonyl)piperazine-1-carboxylate. Using a procedure similar to that for 2-(((6-((1S,2S)-2-(3-chlorophenyl)cyclopropane-1-carboxamido)pyrimidin-4-yl)amino)methyl)-6-cyclopropyl-N,N-dimethylimidazo[1,2-a]pyridine-8-carboxamide, tert-butyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridine-8-carbonyl)piperazine-1-carboxylate was synthesized from 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid (0.23 mmol, 116 mg) and tert-butyl piperazine-1-carboxylate (0.23 mmol, 43 mg) and purified by silica gel column chromatography, eluting with a gradient of 0-20% MeOH in DCM (45 mg, 29%). ESI-MS (M+H)+: 671, ¹H NMR (400 MHz, CDCl₃) δ 8.28 (s, 1H), 8.13-8.09 (m, 1H), 7.90-7.89 (m, 1H), 7.49 (s, 1H), 7.29-7.27 (m, 1H), 7.23-7.19 (m, 2H), 7.10-7.07 (m, 1H), 7.03 (d, J=1.5 Hz, 1H), 7.02-6.98 (m, 1H), 5.73-5.72 (m, 1H), 4.68-4.65 (m, 2H), 3.85-3.82 (m, 2H), 3.60-3.60 (m, 2H), 3.44-3.42 (m, 2H), 3.30-3.29 (m, 2H), 2.62-2.54 (m, 1H), 1.93-1.85 (m, 1H), 1.80-1.69 (m, 2H), 1.47 (s, 9H), 1.41-1.35 (m, 1H), 1.01-0.95 (m, 2H), 0.71-0.66 (m, 2H).

Synthesis of (1S,2S)-2-(3-chlorophenyl)-N-(6-(((6-cyclopropyl-8-(piperazine-1-carbonyl)imidazo[1,2-a]pyridin-2-yl)methyl)amino)pyrimidin-4-yl)cyclopropane-1-carboxamide (I-304). A solution of tert-butyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridine-8-carbonyl)piperazine-1-carboxylate (0.067 mmol, 45 mg) and trifluoroacetic acid (0.18 mL) in DCM (3.4 mL) was stirred at room temperature for 6 h. The mixture was concentrated in vacuo, then applied to an SCX column (eluting with MeOH then NH₃ (2M) in DCM/MeOH. The crude product was then purified by preparative HPLC to give the title compound (8.7 mg, 23%). ESI-MS (M+H)+: 571.3, NMR (400 MHz, DMSO) δ 10.59 (s, 1H), 8.35-8.34 (m, 1H), 8.20-8.17 (m, 1H), 7.88 (br s, 1H), 7.66 (s, 1H), 7.34-7.24 (m, 4H), 7.17-7.13 (m, 1H), 6.95 (d, J=1.5 Hz, 1H), 4.56 (br s, 2H), 3.63-3.58 (m, 2H), 3.10-3.07 (m, 2H), 2.81-2.77 (m, 2H), 2.68-2.65 (m, 2H), 2.43-2.34 (m, 2H), 1.97-1.89 (m, 1H), 1.50-1.37 (m, 2H), 0.95-0.89 (m, 2H), 0.71-0.65 (m, 2H). Exchangeable NH signal not visible.

### Example 305

### Synthesis of (1S,2S)-2-(3-chlorophenyl)-N-(6-(((6-cyclopropyl-8-(2-oxo-1,2-dihydropyridin-3-yl)imidazo[1,2-a]pyridin-2-yl)methyl)amino)pyrimidin-4-yl)cyclopropane-1-carboxamide (I-305)

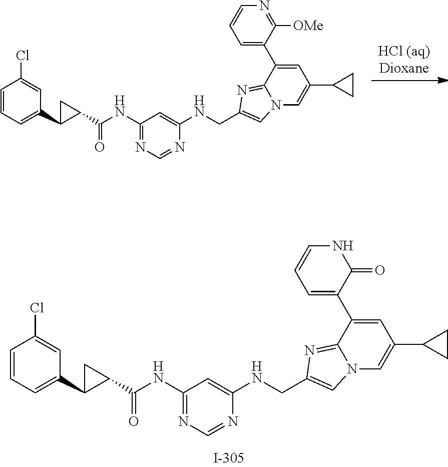 [SMILES: COc1ncccc1-c1cc(C2CC2)cn2cc(CNc3cc(NC(=O)[C@H]4C[C@@H]4c4cccc(Cl)c4)ncn3)nc12 ; "HCl (aq)Dioxane" ; O=C(Nc1cc(NCc2cn3cc(C4CC4)cc(-c4ccc[nH]c4=O)c3n2)ncn1)[C@H]1C[C@@H]1c1cccc(Cl)c1]

A mixture of ((1S,2S)-2-(3-chlorophenyl)-N-(6-(((6-cyclopropyl-8-(2-methoxypyridin-3-yl)imidazo[1,2-a]pyridin-2-yl)methyl)amino)pyrimidin-4-yl)cyclopropane-1-carboxamide) (0.008 mmol, 0.065 g), aqueous HCl (2M, 2.30 mmol, 1.1 mL), and 1,4-dioxane (3 mL) were heated at 80° C. for 2 h. The mixture was cooled to room temperature and concentrated in vacuo. The residue was applied to a SCX column (eluting with 25% MeOH in DCM then 25% (7N NH3 in MeOH) in DCM). The product-containing fractions were concentrated and the residue purified by preparative HPLC to give the title compound (4.7 mg, 7%). ESI-MS (M+H)+: 522.3, ¹H NMR (400 MHz, DMSO) δ 11.86-11.85 (m, 1H), 10.61 (s, 1H), 8.69 (s, 1H), 8.27 (d, J=1.5 Hz, 1H), 8.21 (s, 1H), 7.90-7.87 (m, 2H), 7.66 (s, 1H), 7.45 (d, J=5.8 Hz, 1H), 7.36-7.26 (m, 4H), 7.16 (d, J=7.7 Hz, 1H), 6.39 (dd, J=6.7, 6.7 Hz, 1H), 5.77 (s, 1H), 2.42-2.34 (m, 2H), 1.96-1.90 (m, 1H), 1.49-1.39 (m, 2H), 0.97-0.91 (m, 2H), 0.70-0.65 (m, 2H).

### Example 306

### Synthesis of (1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide (I-306)

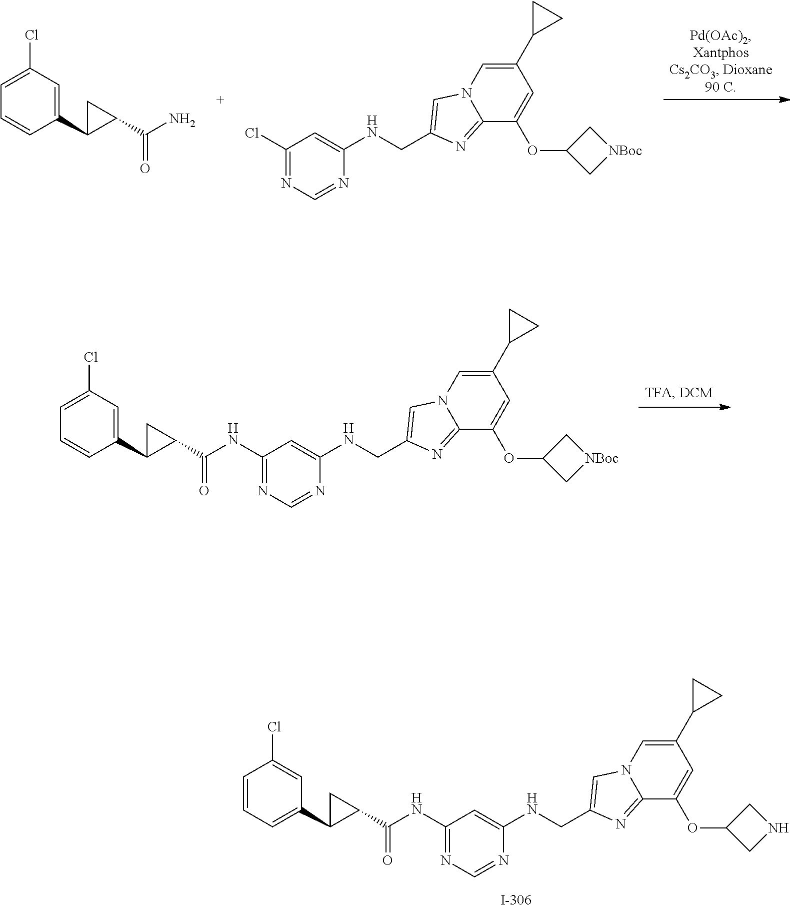

Synthesis of tert-butyl 3-((2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)oxy)azetidine-1-carboxylate. Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using (1S,2S)-2-(3-chlorophenyl)cyclopropane-1-carboxamide (0.23 mmol, 0.046 g) and tert-butyl 3-((2-(((6-chloropyrimidin-4-yl)amino)methyl)-6-cyclopropylimidazo[1,2-a]pyridin-8-yl)methyl)azetidine-1-carboxylate (0.21 mmol; 0.1 g) then purified by silica gel chromatography, eluting with a gradient of 1-20% (7N NH₃ in MeOH) in DCM (0.012 g, 9%).

Synthesis of (1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide (I-306). A solution of (0.019 mmol, 0.012 g) and trifluoroacetic acid (0.5 mL) in DCM (1 mL) was stirred for 30 min, then concentrated in vacuo. The residue was purified by preparative HPLC to give the title compound (4.2 mg, 42%). ESI-MS (M+H)+: 530.3, ¹H NMR (400 MHz, DMSO) δ 10.59 (s, 1H), 8.36 (s, 1H), 8.19 (s, 1H), 7.93-7.90 (m, 2H), 7.59 (s, 1H), 7.34-7.25 (m, 4H), 7.17-7.12 (m, 1H), 6.08 (s, 1H), 5.22-5.16 (m, 1H), 4.54-4.54 (m, 2H), 3.90-3.83 (m, 2H), 3.62 (dd, J=5.8, 8.5 Hz, 2H), 2.43-2.32 (m, 2H), 1.90-1.82 (m, 1H), 1.50-1.37 (m, 2H), 0.91-0.85 (m, 2H), 0.67-0.62 (m, 2H).

### Example 307

### Synthesis of (1S,2S)-2-(3-chlorophenyl)-N-(6-(((6-cyclopropyl-8-(piperazin-1-yl)imidazo[1,2-a]pyridin-2-yl)methyl)amino)pyrimidin-4-yl)cyclopropane-1-carboxamide (I-307)

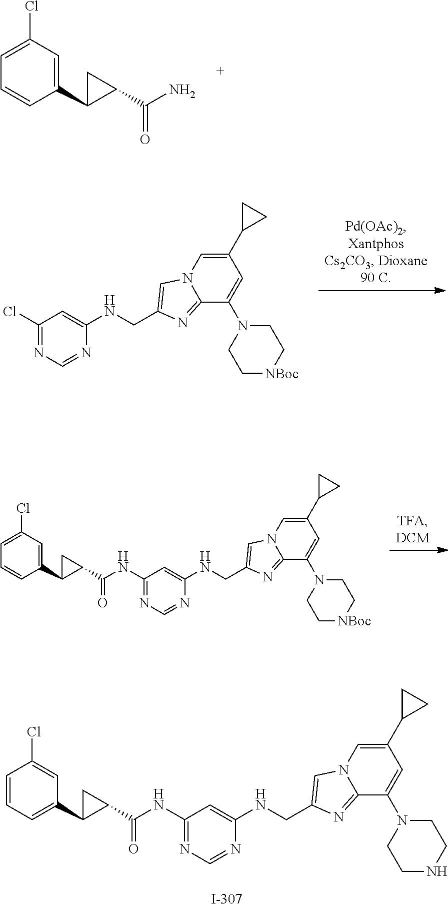

Using a procedure similar to that used for (1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide, the title compound was synthesized from (1S,2S)-2-(3-chlorophenyl)cyclopropane-1-carboxamide and tert-butyl 4-(2-(aminomethyl)-6-cyclopropylimidazo[1,2-a]pyridin-8-yl)piperazine-1-carboxylate, then purified by preparative HPLC (22 mg, 37%) (formic acid salt). ESI-MS (M+H)+: 543.3, ¹H NMR (400 MHz, DMSO) δ 10.59 (s, 1H), 8.27 (0.s, 5H), 8.20-8.18 (m, 1H), 7.87-7.83 (m, 2H), 7.51 (s, 1H), 7.34-7.25 (m, 4H), 7.16-7.13 (m, 1H), 6.14 (d, J=1.1 Hz, 1H), 4.53 (br s, 2H), 3.46-3.41 (m, 4H), 2.96-2.90 (m, 4H), 2.43-2.32 (m, 2H), 1.91-1.81 (m, 1H), 1.50-1.37 (m, 2H), 0.89-0.83 (m, 2H), 0.67-0.62 (m, 2H). Piperazine NH exchangeable not observed.

### Example 308

### Synthesis of (1S,2S)—N-(6-(((8-amino-6-cyclopropylimidazo[1,2-a]pyridin-2-yl)methyl)amino)pyrimidin-4-yl)-2-(3-chlorophenyl)cyclopropane-1-carboxamide (I-308)

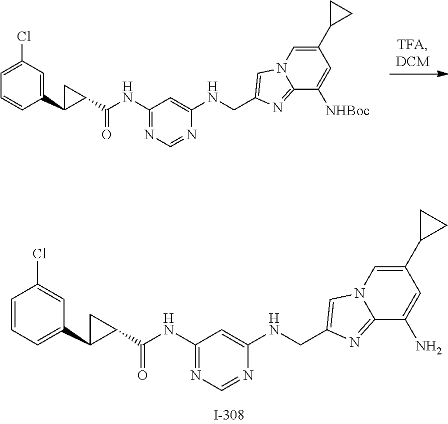

Using a procedure similar to that for (1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide, the title compound was synthesized from tert-butyl (2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)carbamate using trifluoroacetic acid (12 mg, 60%). ESI-MS (M+H)+: 474.3, ¹H NMR (400 MHz, DMSO) δ 10.61 (s, 1H), 8.22 (s, 1H), 7.82-7.81 (m, 1H), 7.60 (d, J=1.0 Hz, 1H), 7.50 (s, 1H), 7.37-7.26 (m, 4H), 7.17 (d, J=7.6 Hz, 1H), 5.98 (d, J=1.3 Hz, 1H), 5.50-5.45 (m, 2H), 4.56-4.56 (m, 2H), 2.47-2.36 (m, 2H), 1.84-1.76 (m, 1H), 1.53-1.39 (m, 2H), 0.89-0.83 (m, 2H), 0.61-0.55 (m, 2H).

### Example 309

### Synthesis of (1S,2S)-2-(3-chlorophenyl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide (I-309)

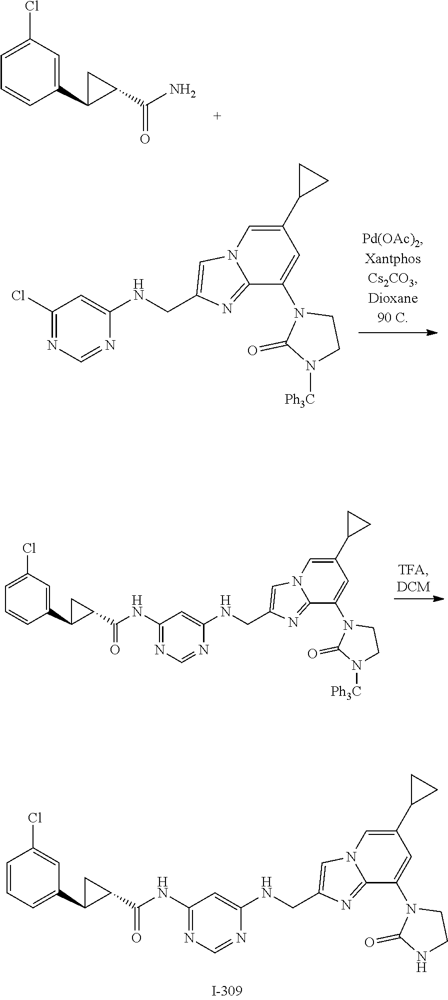

Synthesis of (1S,2S)-2-(3-chlorophenyl)-N-(6-(((6-cyclopropyl-8-(2-oxo-3-tritylimidazolidin-1-yl)imidazo[1,2-a]pyridin-2-yl)methyl)amino)pyrimidin-4-yl)cyclopropane-1-carboxamide. Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using (1S,2S)-2-(3-chlorophenyl)cyclopropane-1-carboxamide (0.19 mmol, 0.04 g) and 1-(2-(((6-chloropyrimidin-4-yl)amino)methyl)-6-cyclopropylimidazo[1,2-a]pyridin-8-yl)-3-tritylimidazolidin-2-one (0.17 mmol; 0.11 g) then purified by silica gel chromatography (eluting with a gradient of 1-10% (7N NH₃ in MeOH) in DCM) (0.117 g, 85%).

Synthesis of (1S,2S)-2-(3-chlorophenyl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide (I-309). A solution of (1S,2S)-2-(3-chlorophenyl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide (0.15 mmol; 0.12 g) and trifluoroacetic acid (1 mL) in DCM (5 mL) was stirred for 1 h the concentrated in vacuo. The residue was passed through an SCX column (eluting with 1:1 MeOH:DCM then 7N NH₃ in MeOH) then purified by silica gel chromatography, eluting with a gradient of 0-100% (7N NH₃ in MeOH) in DCM (0.026 g, 32%). ESE-MS (M+H)+: 543.3, ¹H NMR (400 MHz, DMSO) δ 10.59 (s, 1H), 8.19 (s, 1H), 8.11-8.09 (m, 1H), 7.85 (br s, 1H), 7.61 (s, 1H), 7.34-7.23 (m, 5H), 7.17-7.13 (m, 1H), 6.98-6.95 (m, 1H), 4.55 (br s, 2H), 4.38-4.32 (m, 2H, 3.46-3.40 (m, 2H), 2.44-2.32 (m, 2H), 1.92-1.84 (m, 1H), 1.50-1.43 (m, 1H), 1.43-1.36 (m, 1H), 0.93-0.87 (m, 2H), 0.64-0.59 (m, 2H).

Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the compounds in Table 28 were prepared from 6-chloro-N-((6-cyclopropylimidazo[1,2-a]pyridin-2-yl)methyl)pyrimidin-4-amine and an appropriate coupling partner.

TABLE 28

| | | Coupling Partner / |
|---|---|---|
| Example | Compound | Analytical Data |
| Example 310 | 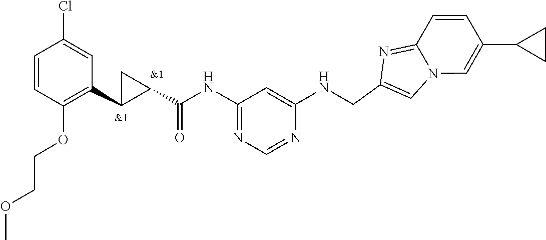 [SMILES: COCCOc1ccc(Cl)cc1[C@H]1C[C@@H]1C(=O)Nc1cc(NCc2cn3cc(C4CC4)ccc3n2)ncn1] | rac-(1S*,2S*)-2-(5-chloro-2-(2- methoxyethoxy)phenyl)cyclopropane- 1-carboxamide ESI-MS (M + H)+: 533.3, ¹H NMR (400 MHz, DMSO): δ, ppm 10.57 (1H, s), 8.31 (1H, t, J = 0.8 Hz), 8.21 (1H, d, J = 0.7 Hz), 8.15 (1H, s), 7.85 (1H, br s), 7.62 (1H, s), 7.38 (1H, d, J = 9.4 Hz), 7.29 (1H, br s), 7.23 (1H, dd, J = 2.6, 8.7 Hz), 7.03 (1H, d, J = 2.6 Hz), 7.01 (1H, d, J = 8.8 Hz), 6.97 (1H, dd, J = 1.8, 9.3 Hz), 4.58 (2H, s), 4.11 (2H, t, J = 4.4 Hz), 3.64 (2H, t, J = 4.4 Hz), 3.23 (3H, s), 2.28 (1H, td, J = 4.9, 8.0 Hz), 1.92 (1H, ddd, J = 5.0, 8.4, 13.4 Hz), 1.40 (2H, ddd, J = 4.1, 9.8, |
| | rac-(1S*,2S*)-2-(5-chloro-2-(2- | 13.8 Hz), 0.92 (2H, ddd, J = 4.3, 6.3, 8.4 |
| | methoxyethoxy)phenyl)-N-(6-(((6- | Hz), 0.67 (2H, ddd, J = 3.2, 4.6, 6.7 Hz); |
| | cyclopropylimidazo[1,2-a]pyridin-2- | CH₂ of cyclopropyl obscured by DMSO |
| | yl)methyl)amino)pyrimidin-4- | |
| | yl)cyclopropane-1-carboxamide, | |
| | formic acid salt (I-310) | |
| Example 311 | 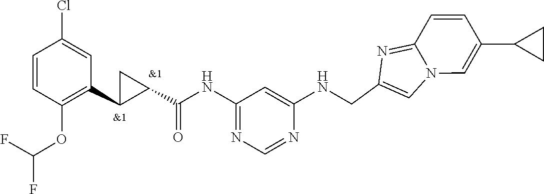 [SMILES: O=C(Nc1cc(NCc2cn3cc(C4CC4)ccc3n2)ncn1)[C@H]1C[C@@H]1c1cc(Cl)ccc1OC(F)F] | rac-(1S*,2S*)-2-(5-chloro-2- (difluoromethoxy)phenyl)cyclopropane- 1-carboxamide ESI-MS (M + H)+: 525.4, ¹H NMR (400 MHz, DMSO) δ 10.60 (s, 1H), 8.32 (s, 1H), 8.27 - 8.21 (m, 2H), 7.92 - 7.83 (m, 1H), 7.64 (s, 1H), 7.39 (d, J = 9.3 Hz, 2H), 7.30 - 7.21 (m, 3H), 7.05 - 6.96 (m, 1H), 4.60 - 4.59 (m, 2H), 2.40 - 2.36 (m, 1H), 1.95 - 1.89 (m, 1H), 1.51 - 1.44 (m, 2H), 0.96 - 0.92 (m, 2H), 0.71 - 0.66 (m, 2H). 1H missing, presumed under |
| | rac-(1S*,2S*)-2-(5-chloro-2- | DMSO peak. |
| | (difluoromethoxy)phenyl)-N-(6-(((6- | |
| | cyclopropylimidazo[1,2-a]pyridin-2- | |
| | yl)methyl)amino)pyrimidin-4- | |
| | yl)cyclopropane-1-carboxamide (I- | |
| | 311) | |
| Example 312 | 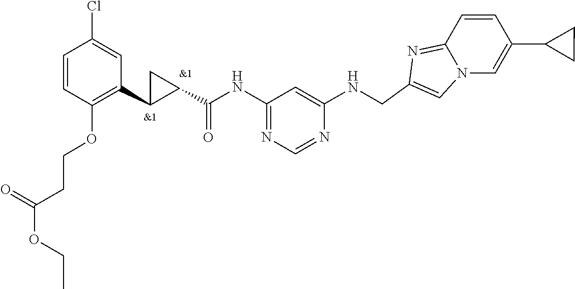 | rac-ethyl 3-(2-((1S*,2S*))-2- carbamoylcyclopropyl)-4- chlorophenoxy)propanoate ESI-MS (M + H)+: 561.3, ¹H NMR (400 MHz, CDCl₃) δ 8.48 - 8.42 (m, 1H), 8.30 (s, 1H), 7.93 (s, 1H), 7.61 - 7.54 (m, 2H), 7.40 (s, 1H), 7.19 - 7.05 (m, 3H), 6.71 - 6.68 (m, 1H), 6.04 - 5.99 (m, OH), 4.79 - 4.66 (m, 4H), 4.35 - 4.22 (m, 2H), 2.67 - 2.61 (m, 1H), 1.98 - 1.84 (m, 2H), 1.76 - 1.70 (m, 1H), 1.46 - 1.40 (m, 1H), 1.31 (t, J = 7.1 Hz, 3H), 1.07 - 1.01 (m, 2H), 0.75 - 0.70 (m, 2H). |
| | rac-ethyl 3-(4-chloro-2-((1S*,2S*)-2- | |
| | ((6-(((6-cyclopropylimidazo[1,2- | |
| | a]pyridin-2- | |
| | yl)methyl)amino)pyrimidin-4- | |
| | yl)carbamoyl)cyclopropyl)phenoxy) | |
| | propanoate (I-312) | |
| Example 313 | 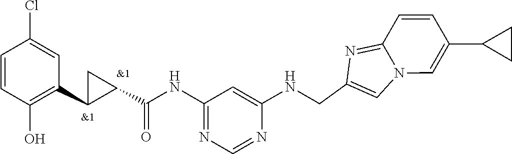 [SMILES: O=C(Nc1cc(NCc2cn3cc(C4CC4)ccc3n2)ncn1)[C@H]1C[C@@H]1c1cc(Cl)ccc1O] | rac-(1S*,2S*)-2-(5-chloro-2- hydroxyphenyl)cyclopropane-1- carboxamide ESI-MS (M + H)+: 475.3, ¹H NMR (400 MHz, DMSO) δ 10.59 (s, 1H), 9.91 (br s, 1H), 8.35 (s, 1H), 8.25 (s, 1H), 7.89 (br s, 1H), 7.66 (s, 1H), 7.42 (d, J = 9.1 Hz, 1H), 7.33 (s, 1H), 7.09 (dd, J = 1.9, 8.5 Hz, 1H), 7.01 (d, J = 9.1 Hz, 1H), 6.95 (s, 1H), 6.84 (d, J = 8.6 Hz, 1H), |
| | rac-(1S*,2S*)-2-(5-chloro-2- | 4.62 (br s, 2H), 2.34 - 2.28 (m, 1H), |
| | hydroxyphenyl)-N-(6-(((6- | 2.00 - 1.92 (m, 1H), 1.45 - 1.37 (m, 2H), |
| | cyclopropylimidazo[1,2-a]pyridin-2- | 0.99 - 0.93 (m, 2H), 0.73 - 0.69 (m, 2H). |
| | yl)methyl)amino)pyrimidin-4- | |
| | yl)cyclopropane-1-carboxamide (I- | |
| | 313) | |
| Example 314 | 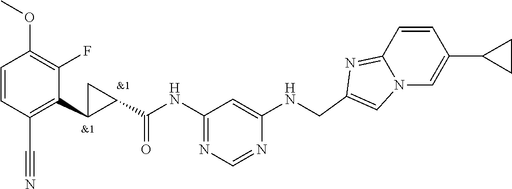 [SMILES: COc1ccc(C#N)c([C@H]2C[C@@H]2C(=O)Nc2cc(NCc3cn4cc(C5CC5)ccc4n3)ncn2)c1F] | rac-(1S*,2S*)-2-(6-cyano-2-fluoro-3- methoxyphenyl)cyclopropane-1- carboxamide ESI-MS (M + H)+: 498.4, ¹H NMR (400 MHz, DMSO) δ 10.78 (s, 1H), 8.50 (s, 1H), 8.35 (s, 1H), 8.26 (s, 1H), 7.91 - 7.91 (m, 1H), 7.75 - 7.65 (m, 2H), 7.45 - 7.27 (m, 3H), 7.04 - 7.00 (m, 1H), 4.63 - 4.58 (m, 2H), 3.97 (s, 3H), 1.99 - 1.92 (m, 1H), 1.60 - 1.53 (m, 2H), 0.99 - 0.93 (m, 2H), 0.74 - 0.68 (m, 2H). 2H missing, presumed under DMSO peak. |
| | rac-(1S*,2S*)-2-(6-cyano-2-fluoro-3- | |
| | methoxyphenyl)-N-(6-(((6- | |
| | cyclopropylimidazo[1,2-a]pyridin-2- | |
| | yl)methyl)amino)pyrimidin-4- | |
| | yl)cyclopropane-1-carboxamide, | |
| | formic acid salt (I-314) | |

### Example 315

### Synthesis of rac-3-(4-chloro-2-((1S*,2S*)-2-((6-(((6-cyclopropylimidazo[1,2-a]pyridin-2-yl)methyl)amino)pyrimidin-4-yl)carbamoyl)cyclopropyl)phenoxy)acetic acid (I-315)

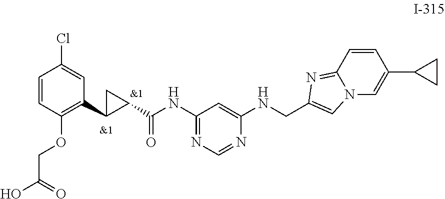 [SMILES: O=C(O)COc1ccc(Cl)cc1[C@H]1C[C@@H]1C(=O)Nc1cc(NCc2cn3cc(C4CC4)ccc3n2)ncn1]

Using a similar procedure to that used for the synthesis of 2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid, the title compound was prepared from rac-ethyl 3-(4-chloro-2-((1S*,2S*)-2-((6-(((6-cyclopropylimidazo[1,2-a]pyridin-2-yl)methyl)amino)pyrimidin-4-yl)carbamoyl)cyclopropyl)phenoxy)acetate (8.8 mg, 68%)(formic acid salt). ESI-MS (M+H)+: 533.3, ¹H NMR (400 MHz, DMSO) δ 10.78 (s, 1H), 8.50 (s, 1H), 8.35 (s, 1H), 8.26 (s, 1H), 7.91-7.91 (m, 1H), 7.75-7.65 (m, 2H), 7.45-7.27 (m, 3H), 7.04-7.00 (m, 1H), 4.63-4.58 (m, 2H), 3.97 (s, 3H), 1.99-1.92 (m, 1H), 1.60-1.53 (m, 2H), 0.99-0.93 (m, 2H), 0.74-0.68 (m, 2H). 2H missing, presumed under DMSO peak.

### Example 316

### Synthesis of rac-(1S*,2S*)-2-(5-chloro-2-(1H-tetrazol-1-yl)phenyl)-N-(6-(((6-cyclopropylimidazo[1,2-a]pyridin-2-yl)methyl)amino)pyrimidin-4-yl)cyclopropane-1-carboxamide (I-316)

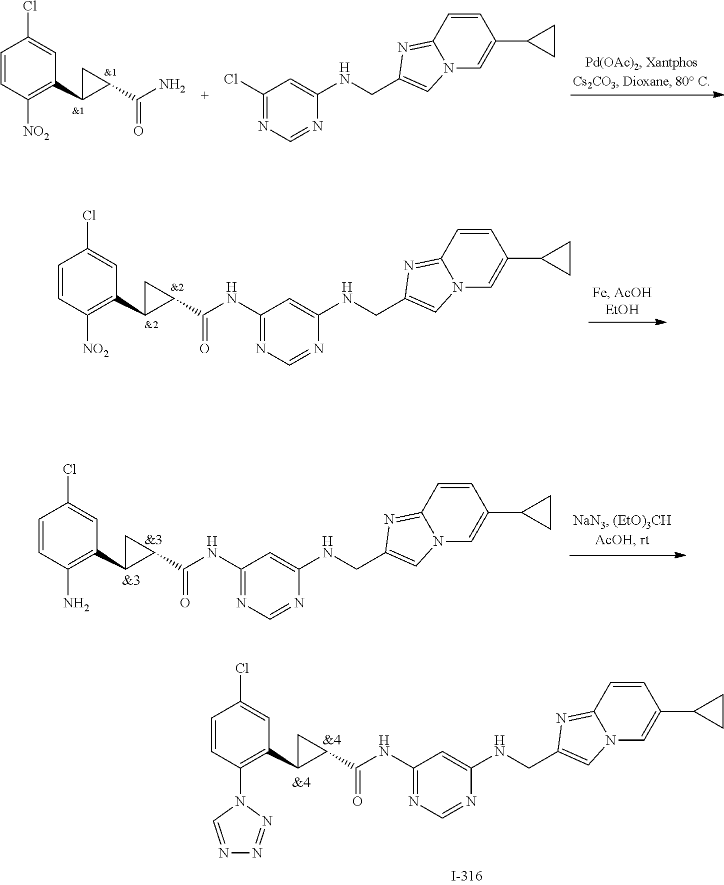 [SMILES: NC(=O)[C@H]1C[C@@H]1c1cc(Cl)ccc1[N+](=O)[O-] ; Clc1cc(NCc2cn3cc(C4CC4)ccc3n2)ncn1 ; O=C(Nc1cc(NCc2cn3cc(C4CC4)ccc3n2)ncn1)[C@H]1C[C@@H]1c1cc(Cl)ccc1[N+](=O)[O-] ; Nc1ccc(Cl)cc1[C@H]1C[C@@H]1C(=O)Nc1cc(NCc2cn3cc(C4CC4)ccc3n2)ncn1 ; O=C(Nc1cc(NCc2cn3cc(C4CC4)ccc3n2)ncn1)[C@H]1C[C@@H]1c1cc(Cl)ccc1-n1cnnn1]

Synthesis of rac-(1S*,2S*)-2-(5-chloro-2-nitrophenyl)-N-(6-(((6-cyclopropylimidazo[1,2-a]pyridin-2-yl)methyl)amino)pyrimidin-4-yl)cyclopropane-1-carboxamide. Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared from 6-chloro-N-((6-cyclopropylimidazo[1,2-a]pyridin-2-yl)methyl)pyrimidin-4-amine and rac-(1S*,2S*)-2-(5-chloro-2-nitrophenyl)cyclopropane-1-carboxamide, then purified by column chromatography on silica using 0-10% (7N NH₃ in MeOH) in DCM (35 mg, ˜50% purity). Used without further purification.

Synthesis of rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide. A mixture of rac-(1S*,2S*)-2-(5-chloro-2-nitrophenyl)-N-(6-(((6-cyclopropylimidazo[1,2-a]pyridin-2-yl)methyl)amino)pyrimidin-4-yl)cyclopropane-1-carboxamide (˜50% purity, 35 mg), iron (9.7 mg, 0.17 mmol), ethanol (2 mL), and acetic acid (0.5 mL) were stirred overnight at 70° C. The mixture was cooled to room temperature, filtered through Celite®, and concentrated in vacuo to give the title compound (20 mg, (˜50% purity) which was used without further purification.

Synthesis of rac-(1S*,2S*)-2-(5-chloro-2-(1H-tetrazol-1-yl)phenyl)-N-(6-(((6-cyclopropylimidazo[1,2-a]pyridin-2-yl)methyl)amino)pyrimidin-4-yl)cyclopropane-1-carboxamide (I-316). rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide (˜50% purity, 20 mg, 0.042 mmol) was dissolved in acetic acid (0.24 mL). Sodium azide (3.4 mg, 0.053 mmol) and triethyl orthoformate (0.022 mL) were added. The mixture was stirred at room temperature overnight. Water (1 mL) was added and product extracted into EtOAc (3×2 mL). The combined organics were passed through a hydrophobic frit and concentrated in vacuo. The residue was purified by preparative HPLC to give the title compound (0.8 mg) as a formic acid salt. ESI-MS (M+H)+: 527.3, ¹H NMR (400 MHz, DMSO) δ 8.50 (s, 2H), 8.32 (s, 1H), 8.23 (s, 1H), 7.69-7.57 (m, 4H), 7.41-7.37 (m, 1H), 7.21 (s, 1H), 7.00-6.96 (m, 1H), 4.59-4.59 (m, 2H), 2.13-2.07 (m, 1H), 1.97-1.89 (m, 1H), 1.40-1.35 (m, 1H), 1.30-1.23 (m, 1H), 0.96-0.90 (m, 2H), 0.71-0.66 (m, 2H). 2H missing, 1H assumed under DMSO and 1H exchangeable.

### Examples 317-321

### Synthesis of (rac-(1S*,2S*)—N-(6-(((6-cyclopropyl-8-(2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide (I-317)

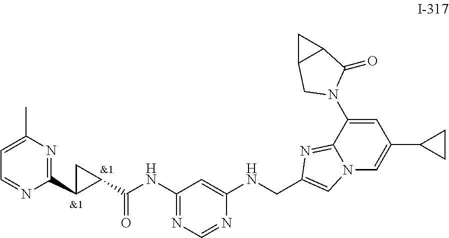 [SMILES: Cc1ccnc([C@H]2C[C@@H]2C(=O)Nc2cc(NCc3cn4cc(C5CC5)cc(N5CC6CC6C5=O)c4n3)ncn2)n1]

Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using 2-(4-methylpyrimidin-2-yl)cyclopropane-1-carboxamide (35 mg, 0.20 mmol) and 3-(2-(((6-chloropyrimidin-4-yl)amino)methyl)-6-cyclopropylimidazo[1,2-a]pyridin-8-yl)-3-azabicyclo[3.1.0]hexan-2-one (71 mg, 0.18 mmol), then purified by preparative LCMS to give the title compound (40 mg, 42%) as a mixture of stereoisomers. ESI-MS (M+H)+: 536, ¹H NMR (400 MHz, DMSO) δ 10.72 (s, 1H), 8.58 (d, J=5.1 Hz, 1H), 8.26 (d, J=1.3 Hz, 1H), 8.24 (s, 1H), 7.92 (s, 1H), 7.69 (s, 1H), 7.35 (s, 1H), 7.26 (d, J=5.3 Hz, 1H), 7.08 (d, J=1.3 Hz, 1H), 4.61 (s, 2H), 4.41-4.36 (m, 1H), 4.05 (d, J=10.4 Hz, 1H), 2.71-2.65 (m, 1H), 2.62-2.57 (m, 1H), 2.47 (s, 3H), 2.19-2.11 (m, 1H), 2.08-2.02 (m, 1H), 1.98-1.91 (m, 1H), 1.63-1.53 (m, 2H), 1.31-1.23 (m, 2H), 0.98-0.93 (m, 2H), 0.92-0.87 (m, 1H), 0.71-0.64 (m, 2H).

The mixture was resolved using SFC (YMC Cellulose-C 4.6×250 mm, 5 μm 40/60 IPA (0.1% DEA)/CO₂, 5.0 mL/min, 120 bar, 40° C.) to give four stereoisomers: (1S,2S)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, (1R,2R)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, (1S,2S)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide, and (1R,2R)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide.

First eluting isomer (I-318, 4.94 mg): ESI-MS (M+H)+: 536, ¹H NMR (400 MHz, DMSO) δ 10.68 (s, 1H), 8.54 (d, J=5.1 Hz, 1H), 8.23 (d, J=1.1 Hz, 1H), 8.20 (s, 1H), 7.88 (s, 1H), 7.65 (s, 1H), 7.31 (s, 1H), 7.22 (d, J=5.4 Hz, 1H), 7.04 (d, J=1.3 Hz, 1H), 4.57 (s, 2H), 4.35 (dd, J=5.9, 10.4 Hz, 1H), 4.01-3.98 (m, 1H), 2.64 (ddd, J=3.7, 5.2, 8.7 Hz, 1H), 2.55 (q, J=2.8 Hz, 1H), 2.43 (s, 3H), 2.15-2.08 (m, 1H), 2.05-1.98 (m, 1H), 1.94-1.87 (m, 1H), 1.58-1.49 (m, 2H), 1.24-1.18 (m, 1H), 0.94-0.84 (m, 3H), 0.66-0.63 (m, 2H). RT=5.99 min, 76.6% e.e.

Second eluting isomer (I-319, 4.59 mg): ESI-MS (M+H)+: 536, ¹H NMR (400 MHz, DMSO) δ 10.68 (s, 1H), 8.52 (d, J=5.1 Hz, 1H), 8.23 (s, 1H), 8.19 (s, 1H), 7.89 (s, 1H), 7.65 (s, 1H), 7.30 (s, 1H), 7.21 (d, J=5.1 Hz, 1H), 7.06 (s, 1H), 4.57 (s, 2H), 4.32 (dd, J=5.9, 10.3 Hz, 1H), 3.97 (d, J=9.4 Hz, 1H), 2.65-2.59 (m, 1H), 2.56-2.52 (m, 1H), 2.41 (s, 3H), 2.14-2.07 (m, 1H), 2.03-1.98 (m, 1H), 1.94-1.86 (m, 1H), 1.57-1.47 (m, 2H), 1.23-1.18 (m, 2H), 0.92-0.85 (m, 3H), 0.64-0.64 (m, 2H). RT=8.32 min, 78.0% e.e.

The third and fourth eluting isomers were not resolved under the above conditions. Further SFC resolution (LUX Cellulose-4 4.6×250 mm, 5 um 55/45 IPA (0.1% DEA)/CO2, 5.0 ml/min, 120 bar, 40° C.) gave:

Third eluting isomer (I-320, 2.97 mg): ESI-MS (M+H)+: 536, ¹H NMR (400 MHz, DMSO) δ 10.69 (s, 1H), 8.54 (d, J=5.1 Hz, 1H), 8.23 (d, J=1.1 Hz, 1H), 8.20 (s, 1H), 7.89 (s, 1H), 7.65 (s, 1H), 7.32 (s, 1H), 7.22 (d, J=5.1 Hz, 1H), 7.04 (d, J=1.5 Hz, 1H), 4.57 (s, 2H), 4.35 (dd, J=5.8, 10.3 Hz, 1H), 4.00 (d, J=10.8 Hz, 1H), 2.64 (ddd, J=3.6, 5.2, 8.5 Hz, 1H), 2.58-2.54 (m, 1H), 2.43 (s, 3H), 2.15-2.08 (m, 1H), 2.04-1.99 (m, 1H), 1.94-1.87 (m, 1H), 1.58-1.49 (m, 2H), 1.26-1.18 (m, 2H), 0.95-0.84 (m, 3H), 0.66-0.63 (m, 2H). RT=8.36 min, 99.9% e.e.

Fourth eluting isomer (I-321, 5.99 mg): ESI-MS (M+H)+: 536, ¹H NMR (400 MHz, DMSO) δ 10.67 (s, 1H), 8.55-8.51 (m, 1H), 8.21 (d, J=1.1 Hz, 1H), 8.18 (s, 1H), 7.87 (s, 1H), 7.63 (s, 1H), 7.30 (s, 1H), 7.21 (d, J=5.1 Hz, 1H), 7.03 (s, 1H), 4.55 (s, 2H), 4.33 (dd, J=5.8, 10.3 Hz, 1H), 3.98 (d, J=10.1 Hz, 1H), 2.62 (ddd, J=3.6, 5.1, 8.4 Hz, 1H), 2.56-2.52 (m, 1H), 2.41 (s, 3H), 2.13-2.06 (m, 1H), 2.02-1.97 (m, 1H), 1.93-1.85 (m, 1H), 1.54-1.47 (m, 2H), 1.22-1.17 (m, 2H), 0.93-0.80 (m, 3H), 0.65-0.61 (m, 2H). RT=11.82 min, 94.4% e.e.

### Examples 322-324

### Synthesis of rac-(1S*,2S*)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-322)

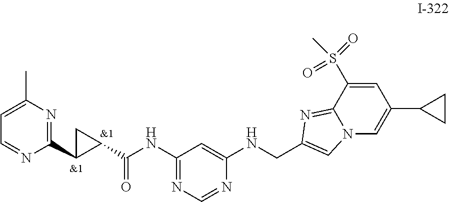 [SMILES: Cc1ccnc([C@H]2C[C@@H]2C(=O)Nc2cc(NCc3cn4cc(C5CC5)cc(S(C)(=O)=O)c4n3)ncn2)n1]

Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using 2-(4-methylpyrimidin-2-yl)cyclopropane-1-carboxamide (35 mg, 0.198) and 6-chloro-N-((6-cyclopropyl-8-(methylsulfonyl)imidazo[1,2-a]pyridin-2-yl)methyl)pyrimidin-4-amine (68 mg, 0.180 mmol), then purified by preparative LCMS to give the title compound (26 mg, 28%) as a mixture of enantiomers. ESI-MS (M+H)+: 519, ¹H NMR (400 MHz, DMSO) δ 10.73 (s, 1H), 8.69 (d, J=1.3 Hz, 1H), 8.58 (d, J=5.1 Hz, 1H), 8.25 (s, 1H), 8.02 (s, 1H), 7.86 (s, 1H), 7.62 (d, J=1.8 Hz, 1H), 7.36 (s, 1H), 7.26 (d, J=5.1 Hz, 1H), 4.70 (s, 2H), 3.59 (s, 3H), 2.71-2.65 (m, 1H), 2.61-2.58 (m, 1H), 2.47 (s, 3H), 2.16-2.08 (m, 1H), 1.63-1.53 (m, 2H), 1.06-1.00 (m, 2H), 0.79-0.74 (m, 2H).

The mixture was resolved using SFC (YMC Cellulose-C 20×250 mm, 5 μm 35/65 MeOH (0.1% DEA)/CO₂, 100 mL/min, 120 bar, 40° C.) to give two enantiomers: (1S,2S)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide and (1R,2R)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide.

First eluting isomer, (1R,2R)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-323): ESI-MS (M+H)+: 519, ¹H NMR (400 MHz, DMSO) δ 10.71 (s, 1H), 8.65 (d, J=1.3 Hz, 1H), 8.54 (d, J=5.1 Hz, 1H), 8.21 (s, 1H), 8.00 (s, 1H), 7.82 (s, 1H), 7.58 (d, J=1.8 Hz, 1H), 7.33 (s, 1H), 7.22 (d, J=5.0 Hz, 1H), 4.66 (s, 2H), 3.55 (s, 3H), 2.67-2.61 (m, 1H), 2.58-2.53 (m, 1H), 2.43 (s, 3H), 2.12-2.04 (m, 1H), 1.58-1.49 (m, 2H), 1.01-0.96 (m, 2H), 0.75-0.70 (m, 2H). RT=2.37 min, 98.2% e.e.

Second eluting isomer, (1S,2S)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide (I-324): ESI-MS (M+H)+: 519, ¹H NMR (400 MHz, DMSO) δ 10.71 (s, 1H), 8.65 (d, J=1.3 Hz, 1H), 8.54 (d, J=5.1 Hz, 1H), 8.21 (s, 1H), 8.00 (s, 1H), 7.82 (s, 1H), 7.58 (d, J=1.8 Hz, 1H), 7.33 (s, 1H), 7.22 (d, J=5.0 Hz, 1H), 4.66 (s, 2H), 3.55 (s, 3H), 2.67-2.61 (m, 1H), 2.58-2.53 (m, 1H), 2.43 (s, 3H), 2.12-2.04 (m, 1H), 1.58-1.49 (m, 2H), 1.01-0.96 (m, 2H), 0.75-0.70 (m, 2H). RT=3.45 min, 99.2% e.e.

Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the compounds in Table 29 were prepared using (1S,2S)-2-(4-chloropyridin-2-yl)cyclopropane-1-carboxamide and an appropriate coupling partner, then purified by preparative LCMS.

TABLE 29

| | | Coupling Partner / |
|---|---|---|
| Example | Compound | Analytical Data |
| Example 325 | 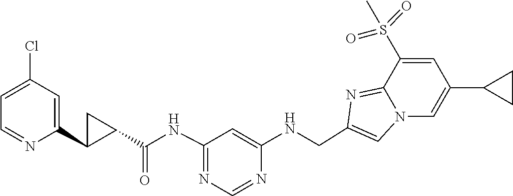 [SMILES: CS(=O)(=O)c1cc(C2CC2)cn2cc(CNc3cc(NC(=O)[C@H]4C[C@@H]4c4cc(Cl)ccn4)ncn3)nc12] | 6-chloro-N-((6-cyclopropyl-8- (methylsulfonyl)imidazo[1,2-a]pyridin-2- yl)methyl)pyrimidin-4-amine ESI-MS (M + H)+: 538.4, ¹H NMR (400 MHz, DMSO) δ 10.68 (s, 1H), 8.64 (s, 1H), 8.44 (d, J = 5.3 Hz, 1H), 8.21 (s, 1H), 8.00 (s, 1H), 7.82 (s, 1H), 7.66 - 7.65 (m, 1H), 7.57 (d, J = 1.4 Hz, 1H), 7.36 (dd, J = 2.0, 5.3 Hz, 1H), 7.32 (s, 1H), 4.70 - 4.63 (m, 2H), 3.56 (s, 3H), 2.64 - 2.57 (m, 1H), 2.12 - 2.06 (m, 1H), 1.57 - 1.47 (m, 3H), 1.02 - 0.96 (m, 2H), 0.75 - 0.70 (m, 2H). |
| | (1S,2S)-2-(4-chloropyridin-2-yl)-N-(6- | |
| | (((6-cyclopropyl-8- | |
| | (methylsulfonyl)imidazo[1,2- | |
| | a]pyridin-2- | |
| | yl)methyl)amino)pyrimidin-4- | |
| | yl)cyclopropane-1-carboxamide (I- | |
| | 325) | |
| Example 326 | 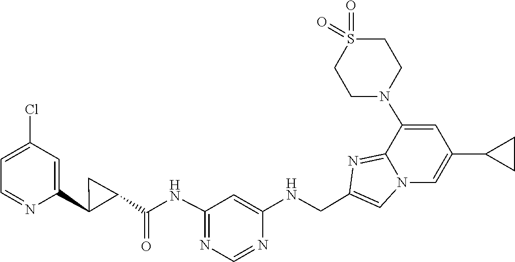 [SMILES: O=C(Nc1cc(NCc2cn3cc(C4CC4)cc(N4CCS(=O)(=O)CC4)c3n2)ncn1)[C@H]1C[C@@H]1c1cc(Cl)ccn1] | 4-(2-(((6-chloropyrimidin-4- yl)amino)methyl)-6- cyclopropylimidazo[1,2-a]pyridin-8- yl)thiomorpholine 1,1-dioxide ESI-MS (M + H)+: 593.4, ¹H NMR (400 MHz, DMSO) δ 10.66 (s, 1H), 8.44 (d, J = 5.3 Hz, 1H), 8.20 (s, 1H), 7.93 (s, 1H), 7.83 (s, 1H), 7.66 (d, J = 1.9 Hz, 1H), 7.57 (s, 1H), 7.36 (dd, J = 2.1, 5.3 Hz, 1H), 7.30 (s, 1H), 6.31 (s, 1H), 4.55 - 4.55 (m, 2H), 4.13 - 4.13 (m, 4H), 3.24 (dd, J = 4.8, 4.8 Hz, 4H), 2.64 - 2.57 (m, 2H), 1.93 - 1.84 (m, 1H), 1.57 - 1.46 (m, 2H), 0.91 - 0.86 (m, 2H), 0.73 - 0.68 (m, 2H). |
| | (1S,2S)-2-(4-chloropyridin-2-yl)-N-(6- | |
| | (((6-cyclopropyl-8-(1,1- | |
| | dioxidothiomorpholino)imidazo[1,2- | |
| | a]pyridin-2- | |
| | yl)methyl)amino)pyrimidin-4- | |
| | yl)cyclopropane-1-carboxamide (I- | |
| | 326) | |
| Example 327 | 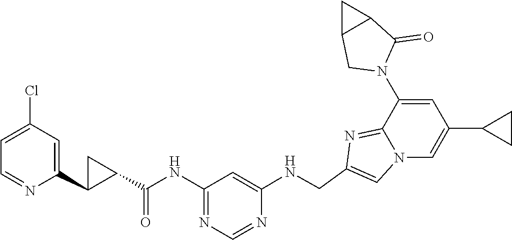 [SMILES: O=C(Nc1cc(NCc2cn3cc(C4CC4)cc(N4CC5CC5C4=O)c3n2)ncn1)[C@H]1C[C@@H]1c1cc(Cl)ccn1] | 3-(2-(((6-chloropyrimidin-4- yl)amino)methyl)-6- cyclopropylimidazo[1,2-a]pyridin-8-yl)-3- azabicyclo[3.1.0]hexan-2-one ESI-MS (M + H)+: 555.3, ¹H NMR (400 MHz, DMSO) δ 10.67 (s, 1H), 8.23 (d, J = 1.2 Hz, 1H), 8.20 (d, J = 0.6 Hz, 1H), 7.89 (s, 1H), 7.66 (d, J = 1.7 Hz, 1H), 7.64 (s, 1H), 7.36 (dd, J = 2.0, 5.4 Hz, 1H), 7.31 (s, 1H), 7.04 (d, J = 1.3 Hz, 1H), 4.35 (ddd, J = 3.4, 5.9, 10.3 Hz, 1H), 4.00 (d, J = 10.0 Hz, 1H), 2.65 - 2.57 (m, 2H), 2.15 - 2.07 (m, 1H), 2.04 - 1.98 (m, 1H), 1.91 (tdd, J = 3.8, 9.2, 9.2 Hz, 1H), 1.57 - 1.47 (m, 2H), 1.21 (ddd, J = 4.0, 4.0, 12.0 Hz, 1H), 0.95 - 0.90 (m, 2H), 0.86 |
| | (1S,2S)-2-(4-chloropyridin-2-yl)-N-(6- | (ddd, J = 1.3, 4.2, 7.3 Hz, 1H), 0.65 (ddd, |
| | (((6-cyclopropyl-8-(2-oxo-3- | J = 1.9, 4.3, 7.1 Hz, 2H). |
| | azabicyclo[3.1.0]hexan-3- | |
| | yl)imidazo[1,2-a]pyridin-2- | |
| | yl)methyl)amino)pyrimidin-4- | |
| | yl)cyclopropane-1-carboxamide (I- | |
| | 327) | |

### Example 328

### Synthesis of (1S,2S)-2-(4-chloropyridin-2-yl)-N-(6-(((6-cyclopropylimidazo[1,2-a]pyridin-2-yl)methyl)(2-hydroxyethyl)amino)pyrimidin-4-yl)cyclopropane-1-carboxamide (I-328)

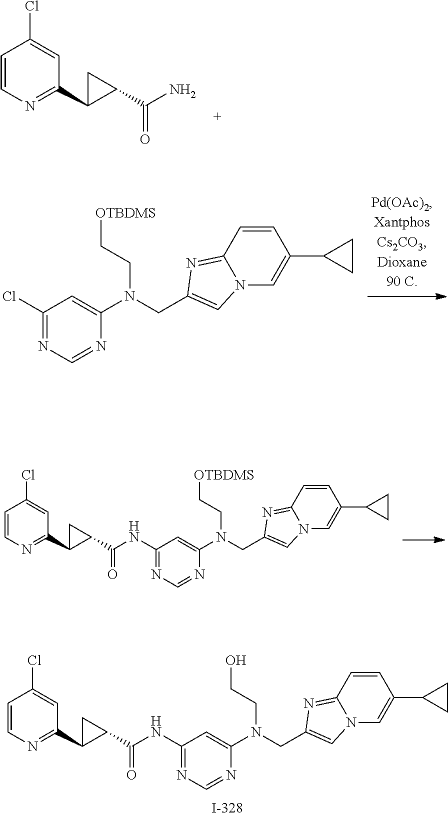 [SMILES: NC(=O)[C@H]1C[C@@H]1c1cc(Cl)ccn1 ; CC(C)(C)[Si](C)(C)OCCN(Cc1cn2cc(C3CC3)ccc2n1)c1cc(Cl)ncn1 ; CC(C)(C)[Si](C)(C)OCCN(Cc1cn2cc(C3CC3)ccc2n1)c1cc(NC(=O)[C@H]2C[C@@H]2c2cc(Cl)ccn2)ncn1 ; O=C(Nc1cc(N(CCO)Cc2cn3cc(C4CC4)ccc3n2)ncn1)[C@H]1C[C@@H]1c1cc(Cl)ccn1]

Synthesis of (1S,2S)—N-(6-((2-((tert-butyldimethylsilyl)oxy)ethyl)((6-cyclopropylimidazo[1,2-a]pyridin-2-yl)methyl)amino)pyrimidin-4-yl)-2-(4-chloropyridin-2-yl)cyclopropane-1-carboxamide. Using a similar procedure to that used for (1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide, the title compound was prepared using (1S,2S)-2-(4-chloropyridin-2-yl)cyclopropane-1-carboxamide (0.14 mmol, 0.028 g) and N-(2-((tert-butyldimethylsilyl)oxy)ethyl)-6-chloro-N-((6-cyclopropylimidazo[1,2-a]pyridin-2-yl)methyl)pyrimidin-4-amine (0.13 mmol; 0.060 g), then purified by silica gel chromatography (eluting with a gradient of 1-20% (7N NH₃ in MeOH) in DCM) (0.030 g, 37%). ESI-MS (M+H)+: 618.4

Synthesis of (1S,2S)-2-(4-chloropyridin-2-yl)-N-(6-(((6-cyclopropylimidazo[1,2-a]pyridin-2-yl)methyl)(2-hydroxyethyl)amino)pyrimidin-4-yl)cyclopropane-1-carboxamide (I-328). Using a similar procedure to that for (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide, the title compound was synthesized from (1S,2S)—N-(6-((2-((tert-butyldimethylsilyl)oxy)ethyl)((6-cyclopropylimidazo[1,2-a]pyridin-2-yl)methyl)amino)pyrimidin-4-yl)-2-(4-chloropyridin-2-yl)cyclopropane-1-carboxamide (8 mg, 33%). ESI-MS (M+H)+: 502.4, ¹H NMR (400 MHz, DMSO) δ 10.75-10.72 (m, 1H), 8.43 (d, J=5.4 Hz, 1H), 8.33-8.30 (m, 1H), 8.27-8.25 (m, 1H), 7.66-7.63 (m, 2H), 7.45-7.34 (m, 3H), 7.02-6.99 (m, 1H), 5.21-5.20 (m, 1H), 4.95-4.69 (m, 2H), 3.80-3.55 (m, 4H), 2.62-2.55 (m, 2H), 1.97-1.89 (m, 1H), 1.56-1.48 (m, 2H), 0.95-0.90 (m, 2H), 0.71-0.65 (m, 2H).

### Examples 329-333

### Synthesis of rac-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide (I-329)

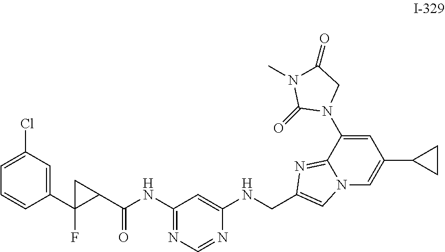 [SMILES: CN1C(=O)CN(c2cc(C3CC3)cn3cc(CNc4cc(NC(=O)C5CC5(F)c5cccc(Cl)c5)ncn4)nc23)C1=O]

Using a similar procedure to that used for (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide, the title compound was prepared, as a mixture of stereoisomers, using 2-(3-chlorophenyl)-2-fluorocyclopropane-1-carboxamide (67 mg, 0.31 mmol) and 1-(2-(((6-chloropyrimidin-4-yl)amino)methyl)-6-cyclopropylimidazo[1,2-a]pyridin-8-yl)-3-methylimidazolidine-2,4-dione (118 mg, 0.29 mmol). The mixture was further purified by preparative LCMS to give two mixtures of enantiomers: (1R,2R)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, (1S,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, (1R,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide, and (1S,2R)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide.

First eluting mixture of enantiomers, (I-330): ESI-MS (M+H)+: 589.4, ¹H NMR (400 MHz, DMSO) δ 10.79 (s, 1H), 8.24-8.19 (m, 2H), 7.84-7.79 (m, 1H), 7.66 (s, 1H), 7.52 (s, 1H), 7.46-7.40 (m, 3H), 7.34 (s, 1H), 7.04 (s, 1H), 4.89-4.86 (m, 2H), 4.59-4.51 (m, 2H), 2.99-2.98 (m, 4H), 2.09-1.85 (m, 3H), 0.98-0.92 (m, 2H), 0.68-0.62 (m, 2H).

Second eluting mixture of enantiomers (I-331): ESI-MS (M+H)+: 589.3, ¹H NMR (400 MHz, DMSO) δ 10.78 (s, 1H), 8.29 (s, 1H), 8.22 (s, 1H), 8.01-7.91 (m, 1H), 7.76-7.73 (m, 1H), 7.50-7.47 (m, 3H), 7.41-7.34 (m, 3H), 4.91 (s, 2H), 4.64-4.61 (m, 2H), 3.00-2.98 (m, 3H), 2.76-2.68 (m, 1H), 2.25-2.14 (m, 1H), 2.00-1.93 (m, 1H), 1.85-1.77 (m, 1H), 1.00-0.93 (m, 2H), 0.70-0.64 (m, 2H).

The second eluting mixture was then further separated by SFC (YMC Cellulose-C 4.6×250 mm, 5 μm 50/50 MeOH (0.1% DEA)/CO₂, 5.0 mL/min, 120 bar, 40° C.) to give two enantiomers: First eluting enantiomer (I-332): ESI-MS (M+H)+: 589.3, ¹H NMR (400 MHz, DMSO) δ 10.79 (s, 1H), 8.30-8.24 (m, 2H), 7.98-7.90 (m, 1H), 7.75 (s, 1H), 7.54-7.50 (m, 3H), 7.44-7.38 (m, 3H), 4.98-4.96 (m, 2H), 4.64-4.61 (m, 2H), 3.03 (s, 3H), 2.81-2.72 (m, 1H), 2.29-2.18 (m, 1H), 2.03-1.95 (m, 1H), 1.89-1.80 (m, 1H), 1.04-0.96 (m, 2H), 0.72-0.67 (m, 2H), RT=4.18 min, 99.9% e.e.

Second eluting enantiomer (I-333): ESI-MS (M+H)+: 589.3, ¹H NMR (400 MHz, DMSO) δ 10.79 (s, 1H), 8.30-8.24 (m, 2H), 7.99-7.92 (m, 1H), 7.75 (s, 1H), 7.54-7.50 (m, 3H), 7.44-7.37 (m, 3H), 4.98-4.96 (m, 2H), 4.66-4.64 (m, 2H), 3.03 (s, 3H), 2.81-2.64 (m, 2H), 2.29-2.18 (m, 1H), 2.03-1.95 (m, 1H), 1.88-1.80 (m, 1H), 1.02-0.96 (m, 2H), 0.72-0.67 (m, 2H), RT=5.57 min, 99.2% e.e.

### Examples 334-336

### Synthesis of (rac-(1S*,2S*))-1-(2-(((6-chloropyrimidin-4-yl)amino)methyl)-6-cyclopropylimidazo[1,2-a]pyridin-8-yl)pyrrolidin-2-one (I-334)

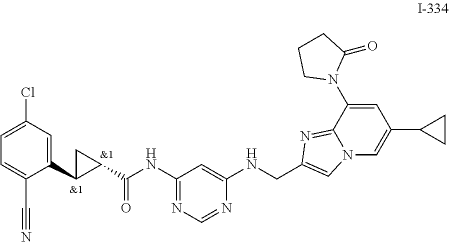 [SMILES: N#Cc1ccc(Cl)cc1[C@H]1C[C@@H]1C(=O)Nc1cc(NCc2cn3cc(C4CC4)cc(N4CCCC4=O)c3n2)ncn1]

Using a similar procedure to that used for (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide, the following compound was prepared using (rac-(1S*,2S*))-2-(5-chloro-2-cyanophenyl)cyclopropane-1-carboxamide (89 mg, 0.40 mmol) and 1-(2-(((6-chloropyrimidin-4-yl)amino)methyl)-6-cyclopropylimidazo[1,2-a]pyridin-8-yl)pyrrolidin-2-one (140 mg, 0.37 mmol), then purified by preparative LCMS to give the title compound (12 mg, 6%) as a mixture of enantiomers. ESI-MS (M+H)+: 567.2, ¹H NMR (400 MHz, DMSO) δ 10.69 (s, 1H), 8.26 (s, 1H), 8.22 (s, 1H), 7.96 (s, 1H), 7.88 (d, J=8.3 Hz, 1H), 7.67 (s, 1H), 7.54 (dd, J=1.9, 8.3 Hz, 1H), 7.43 (s, 1H), 7.32 (s, 1H), 7.13 (s, 1H), 4.59-4.59 (m, 2H), 4.16 (dd, J=7.0, 7.0 Hz, 2H), 2.62-2.55 (m, 1H), 2.50-2.46 (m, 2H), 2.18-2.09 (m, 2H), 1.97-1.88 (m, 1H), 1.65-1.54 (m, 2H), 0.96-0.90 (m, 2H), 0.68-0.62 (m, 2H).

The mixture was then separated by SFC (YMC Cellulose-C 10×250 mm, 5 μm 55/45 MeOH (0.1% DEA)/CO₂, 15 ml/min, 120 bar, 40° C.) to give two enantiomers: (1S,2S)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide and (1R,2R)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide.

First eluting isomer (I-335): ESI-MS (M+H)+: 567.3, ¹H NMR (400 MHz, DMSO) δ 10.69 (s, 1H), 8.26 (s, 1H), 8.22 (s, 1H), 7.96 (s, 1H), 7.88 (d, J=8.3 Hz, 1H), 7.67 (s, 1H), 7.54 (dd, J=1.9, 8.3 Hz, 1H), 7.43 (s, 1H), 7.32 (s, 1H), 7.13 (s, 1H), 4.59-4.59 (m, 2H), 4.16 (dd, J=7.0, 7.0 Hz, 2H), 2.62-2.55 (m, 1H), 2.50-2.46 (m, 2H), 2.18-2.09 (m, 2H), 1.97-1.88 (m, 1H), 1.65-1.54 (m, 2H), 0.96-0.90 (m, 2H), 0.68-0.62 (m, 2H). 1.65 min, RT=1.65 min, 99.9% e.e

Second eluting isomer (I-336): ESI-MS (M+H)+: 567.2, ¹H NMR (400 MHz, DMSO) δ 10.69 (s, 1H), 8.26 (s, 1H), 8.22 (s, 1H), 7.96 (s, 1H), 7.88 (d, J=8.3 Hz, 1H), 7.67 (s, 1H), 7.54 (dd, J=1.9, 8.3 Hz, 1H), 7.43 (s, 1H), 7.32 (s, 1H), 7.13 (s, 1H), 4.59-4.59 (m, 2H), 4.16 (dd, J=7.0, 7.0 Hz, 2H), 2.62-2.55 (m, 1H), 2.50-2.46 (m, 2H), 2.18-2.09 (m, 2H), 1.97-1.88 (m, 1H), 1.65-1.54 (m, 2H), 0.96-0.90 (m, 2H), 0.68-0.62 (m, 2H). 3.86 min, RT=3.86 min, 99.9% e.e.

### Example 337

### Synthesis of (rac-(1S*,2S*))-2-(5-chloro-2-cyanophenyl)-N-(6-(((6-cyclopropyl-8-(2-hydroxypropan-2-yl)imidazo[1,2-a]pyridin-2-yl)methyl)amino)pyrimidin-4-yl)cyclopropane-1-carboxamide (I-337)

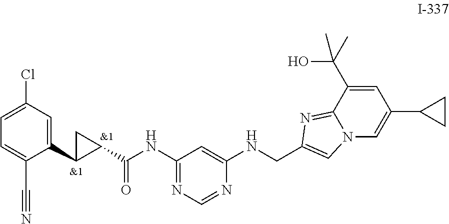 [SMILES: CC(C)(O)c1cc(C2CC2)cn2cc(CNc3cc(NC(=O)[C@H]4C[C@@H]4c4cc(Cl)ccc4C#N)ncn3)nc12]

Using a similar procedure to that used for (1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide, the title compound was prepared using (rac-(1S*,2S*))-2-(5-chloro-2-cyanophenyl)cyclopropane-1-carboxamide (92 mg, 0.41 mmol) and 2-(2-(aminomethyl)-6-cyclopropylimidazo[1,2-a]pyridin-8-yl)propan-2-ol (135 mg, 0.38 mmol), then purified by silica gel chromatography, eluting with a gradient of 0-10% MeOH in DCM (36 mg, 18%) as a mixture of enantiomers. ESI-MS (M+H)+: 542.3, ¹H NMR (400 MHz, DMSO) δ 10.67 (s, 1H), 8.22 (s, 1H), 8.18 (d, J=1.3 Hz, 1H), 7.91-7.87 (m, 2H), 7.59 (s, 1H), 7.54 (dd, J=2.1, 8.3 Hz, 1H), 7.42 (d, J=1.9 Hz, 1H), 7.33 (s, 1H), 7.06 (d, J=1.8 Hz, 1H), 5.52 (s, 1H), 4.59-4.59 (m, 2H), 2.64-2.57 (m, 1H), 2.50-2.47 (m, 1H), 1.96-1.88 (m, 1H), 1.66 (s, 6H), 1.64-1.54 (m, 2H), 0.94-0.89 (m, 2H), 0.68-0.63 (m, 2H).

### Example 338

### Synthesis of (1S,2S)-2-(3-chlorophenyl)-N-(4-(((6-cyclopropylimidazo[1,2-a]pyridin-2-yl)methyl)amino)-6-methylpyridin-2-yl)cyclopropane-1-carboxamide (I-338)

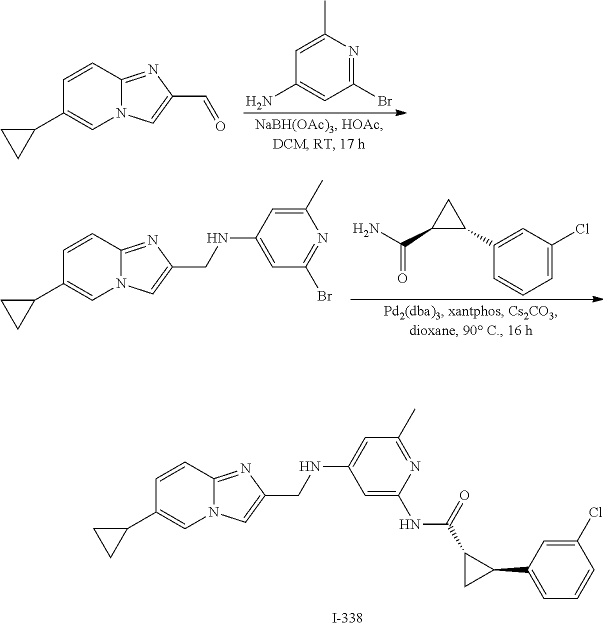

Synthesis of 2-bromo-N-((6-cyclopropylimidazo[1,2-a]pyridin-2-yl)methyl)-6-methylpyridin-4-amine. A mixture of 6-cyclopropylimidazo[1,2-a]pyridine-2-carbaldehyde (200 mg, 1.07 mmol), 2-bromo-6-methylpyridin-4-amine (187 mg, 1.0 mmol), and HOAc (10 drops) in DCM (5 mL) was stirred at room temperature for 1 h under N₂. Then the NaBH(OAc)₃ (318 mg, 1.5 mmol) was added to the mixture and stirred for 16 h. The mixture was diluted with DCM (20 mL) and then extracted with water (3×5 mL). The organic layer was concentrated in vacuo and purified by Prep-TLC (DCM/MeOH=15/1) to give 2-bromo-N-((6-cyclopropylimidazo[1,2-a]pyridin-2-yl)methyl)-6-methylpyridin-4-amine as a light yellow oil (140 mg, 36%). ESI-MS [M+H]⁺: 357.1.

The compounds in Table 30 were prepared in a similar manner to 2-bromo-N-((6-cyclopropylimidazo[1,2-a]pyridin-2-yl)methyl)-6-methylpyridin-4-amine from 6-cyclopropylimidazo[1,2-a]pyridine-2-carbaldehyde and an appropriate aminopyridine.

TABLE 30

| Structure | Analytical Data |
|---|---|
| 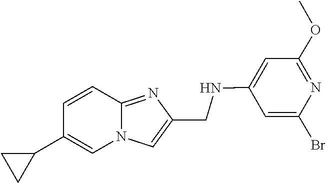 [SMILES: COc1cc(NCc2cn3cc(C4CC4)ccc3n2)cc(Br)n1] | ESI-MS [M + H]+: 373.2. |
| 2-bromo-N-((6-cyclopropylimidazo[1,2-a]pyridin-2-yl)methyl)-6- | |
| methoxypyridin-4-amine | |
| 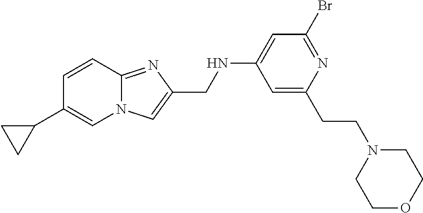 [SMILES: Brc1cc(NCc2cn3cc(C4CC4)ccc3n2)cc(CCN2CCOCC2)n1] | ESI-MS [M + H]+: 456.1 |
| 2-bromo-N-((6-cyclopropylimidazo[1,2-a]pyridin-2-yl)methyl)-6-(2- | |
| morpholinoethyl)pyridin-4-amine | |
| 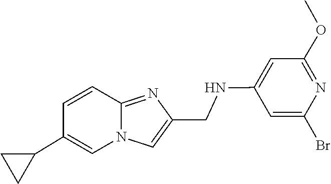 [SMILES: COc1cc(NCc2cn3cc(C4CC4)ccc3n2)cc(Br)n1] | ESI-MS [M + H]+: 373.1. |
| 2-bromo-N-((6-cyclopropylimidazo[1,2-a]pyridin-2-yl)methyl)-6- | |
| methoxypyridin-4-amine | |

Synthesis of (1S,2S)-2-(3-chlorophenyl)-N-(4-(((6-cyclopropylimidazo[1,2-a]pyridin-2-yl)methyl)amino)-6-methylpyridin-2-yl)cyclopropane-1-carboxamide (I-338). A mixture of 2-bromo-N-((6-cyclopropylimidazo[1,2-a]pyridin-2-yl)methyl)-6-methylpyridin-4-amine (45 mg, 0.12 mmol), (1S,2S)-2-(3-chlorophenyl)cyclopropane-1-carboxamide (23.4 mg, 0.12 mmol), Pd₂(dba)₃ (11 mg, 0.012 mmol), xantphos (7 mg, 0.012 mmol), and Cs₂CO₃ (98 mg, 0.23 mmol) in 1,4-dioxane (5 mL) was stirred at 90° C. for 16 h under N₂. The mixture was cooled to room temperature and concentrated in vacuo. The residue was purified by Prep-HPLC to give (1S,2S)-2-(3-chlorophenyl)-N-(4-(((6-cyclopropylimidazo[1,2-a]pyridin-2-yl)methyl) amino)-6-methylpyridin-2-yl)cyclopropane-1-carboxamide (6 mg, 11%) as a light yellow solid. ESI-MS [M+H]+: 472.2, ¹H NMR (400 MHz, DMSO) δ 10.32 (s, 1H), 8.31 (s, 1H), 7.61 (s, 1H), 7.38 (d, J=9.4 Hz, 1H), 7.31-7.23 (m, 4H), 7.13 (d, J=7.7 Hz, 1H), 7.00-6.94 (m, 2H), 6.20 (s, 1H), 4.34 (d, J=5.7 Hz, 2H), 2.37-2.33 (m, 2H), 2.17 (s, 3H), 1.94-1.89 (m, 1H), 1.45-1.40 (m, 1H), 1.34-1.30 (m, 1H), 0.93-0.88 (m, 2H), 0.68-0.64 (m, 2H).

The compounds in Table 31 were prepared in a similar manner to (1S,2S)-2-(3-chlorophenyl)-N-(4-(((6-cyclopropylimidazo[1,2-a]pyridin-2-yl)methyl)amino)-6-methylpyridin-2-yl)cyclopropane-1-carboxamide from a cyclopropyl amide and an appropriate coupling partner.

TABLE 31

| | | Coupling Partner / |
|---|---|---|
| Example | Structure | Analytical Data |
| Example 339 | 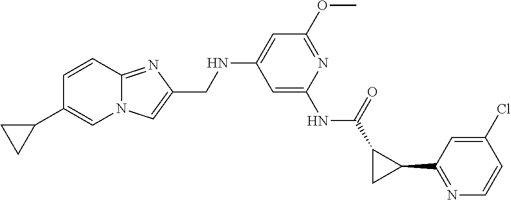 [SMILES: COc1cc(NCc2cn3cc(C4CC4)ccc3n2)cc(NC(=O)[C@H]2C[C@@H]2c2cc(Cl)ccn2)n1] | 2-bromo-N-((6- cyclopropylimidazo[1,2-a]pyridin-2- yl)methyl)-6-methoxypyridin-4-amine ESI-MS [M + H]+: 489.2. ¹H NMR (400 MHz, DMSO) δ 10.17 (s, 1H), 8.42 (d, J = 5.3 Hz, 1H), 8.31 (s, 1H), 7.64 - 7.60 (m, 2H), 7.40 - 7.31 (m, 2H), 7.14 (s, 1H), 7.09 - 7.02 (m, 1H), 6.96 (dd, J = 9.3, 1.6 Hz, 1H), 5.63 (s, 1H), 4.32 (d, J = 5.7 Hz, 2H), 3.68 (s, 3H), 2.62 - 2.57 (m, 1H), 2.56 - 2.52 (m, 1H), 1.96 - 1.85 (m, 1H), 1.53 - 1.40 (m, 2H), 0.95 - 0.86 (m, 2H), 0.72 - |
| | (1S,2S)-2-(4-chloropyridin-2-yl)-N-(4- | 0.60 (m, 2H). |
| | (((6-cyclopropylimidazo[1,2-a]pyridin- | |
| | 2-yl)methyl)amino)-6-methoxypyridin- | |
| | 2-yl)cyclopropane-1-carboxamide (I- | |
| | 339) | |
| Example 340 | 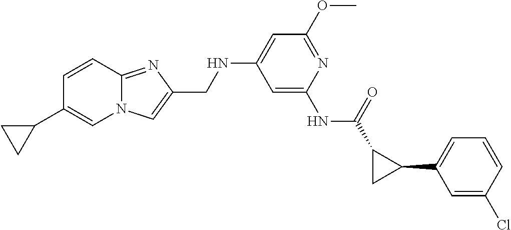 [SMILES: COc1cc(NCc2cn3cc(C4CC4)ccc3n2)cc(NC(=O)[C@H]2C[C@@H]2c2cccc(Cl)c2)n1] | 2-bromo-N-((6- cyclopropylimidazo[1,2-a]pyridin-2- yl)methyl)-6-methoxypyridin-4-amine ESI-MS [M + H]+: 488.2. ¹H NMR (400 MHz, DMSO) δ 10.10 (s, 1H), 8.31 (s, 1H), 8.18 (s, 1H), 7.61 (s, 1H), 7.48 - 7.21 (m, 4H), 7.15 (d, J = 6.1 Hz, 2H), 7.06 (s, 1H), 6.98 - 6.95 (m, 1H), 5.64 (s, 1H), 4.32 (d, J = 5.6 Hz, 2H), 3.69 (s, 3H), 2.46 - 2.30 (m, 2H), 1.98 - 1.81 (m, 1H), 1.55 - 1.40 (m, 1H), 1.35- 1.30 (m, 1H), 0.99 - 0.80 (m, 2H), 0.76 - 0.54 (m, 2H). |
| | (1S,2S)-2-(3-chlorophenyl)-N-(4-(((6- | |
| | cyclopropylimidazo[1,2-a]pyridin-2- | |
| | yl)methyl)amino)-6-methoxypyridin-2- | |
| | yl)cyclopropane-1-carboxamide (I-340) | |
| Example 341 | 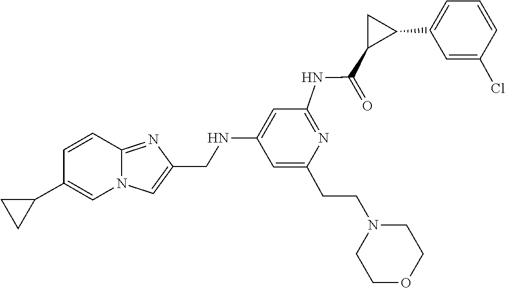 [SMILES: O=C(Nc1cc(NCc2cn3cc(C4CC4)ccc3n2)cc(CCN2CCOCC2)n1)[C@H]1C[C@@H]1c1cccc(Cl)c1] | 2-bromo-N-((6- cyclopropylimidazo[1,2-a]pyridin-2- yl)methyl)-6-(2- morpholinoethyl)pyridin-4-amine ESI-MS [M + H]+: 571.2, ¹H NMR (400 MHz, DMSO) δ 10.32 (s, 1H), 8.31 (s, 1H), 7.62 (s, 1H), 7.37 (d, J = 9.3 Hz, 1H), 7.33 - 7.23 (m, 4H), 7.14 - 7.07 (m, 2H), 6.98 - 6.95 (m, 1H), 6.26 (s, 1H), 4.35 (d, J = 5.6 Hz, 2H), 3.56 - 3.51 (m, 4H), 2.67 - 2.62 (m, 4H), 2.45 - 2.32 (m, 6H), 1.94 - 1.87 (m, 1H), 1.46 - 1.41 (m, 1H), 1.34 - 1.30 (m, 1H), 0.93 - 0.88 (m, 2H), 0.68 - 0.64 (m, 2H). |
| | (1S,2S)-2-(3-chlorophenyl)-N-(4-(((6- | |
| | cyclopropylimidazo[1,2-a]pyridin-2- | |
| | yl)methyl)amino)-6-(2- | |
| | morpholinoethyl)pyridin-2- | |
| | yl)cyclopropane-1-carboxamide (I-341) | |
| Example 342 | 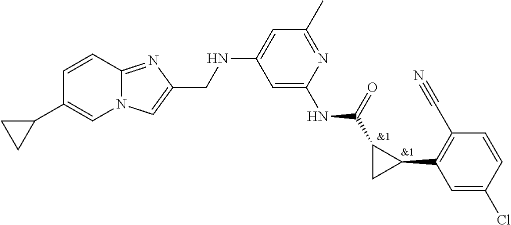 | 2-bromo-N-((6- cyclopropylimidazo[1,2-a]pyridin-2- yl)methyl)-6-methylpyridin-4-amine ESI-MS [M + H]+: 497.2, ¹H NMR (400 MHz, DMSO) δ 10.36 (s, 1H), 8.30 (s, 1H), 8.23 (s, 1H), 7.85 (d, J = 8.3 Hz, 1H), 7.62 (s, 1H), 7.53 - 7.50 (m, 2.0 Hz, 1H), 7.39 - 7.37 (m, 2H), 7.30 (s, 1H), 7.03 - 7.00 (m, 1H), 6.98 - 6.95 (m, 1.7 Hz, 1H), 6.20 (s, 1H), 4.35 (d, J = 5.7 Hz, 2H), 2.57 - 2.54 (m, 2H), 2.18 (s, 3H), 1.94 - 1.87 (m, 1H), 1.51 - 1.49 (m, 2H), 0.93 - 0.88 (m, 2H), 0.68 - 0.64 (m, 2H). |
| | rac-(1S*,2S*)-2-(5-chloro-2- | |
| | cyanophenyl)-N-(4-(((6- | |
| | cyclopropylimidazo[1,2-a]pyridin-2- | |
| | yl)methyl)amino)-6-methylpyridin-2- | |
| | yl)cyclopropane-1-carboxamide (I-342) | |

### Example 343

### Synthesis of (1S,2S)-2-(4-chloropyridin-2-yl)-N-(4-(((6-cyclopropylimidazo[1,2-a]pyridin-2-yl)methyl)amino)-6-oxo-1,6-dihydropyridin-2-yl)cyclopropane-1-carboxamide (I-343)

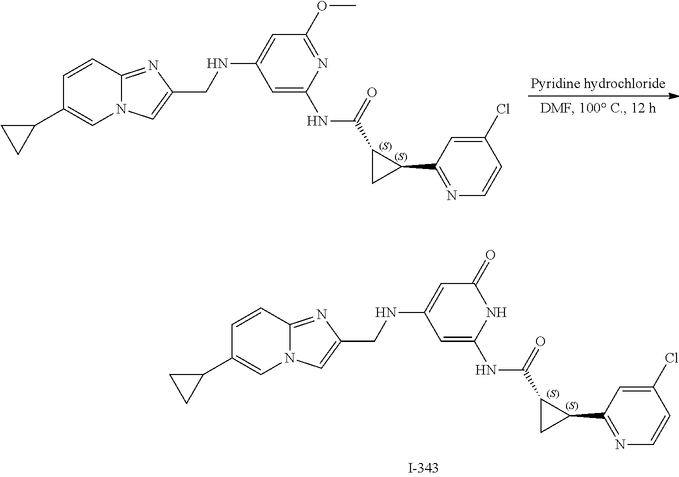

A mixture of (1S,2S)-2-(4-chloropyridin-2-yl)-N-(4-(((6-cyclopropylimidazo[1,2-a]pyridin-2-yl)methyl)amino)-6-methoxypyridin-2-yl)cyclopropane-1-carboxamide (70 mg, 0.14 mmol) and pyridine hydrochloride (800 mg, 6.9 mmol) in DMF (15 mL) was stirred at 100° C. for 12 h. After cooling to room temperature, the mixture was diluted with water (100 mL) and extracted with EtOAc (3×50 mL). The combined extract was washed with brine (50 mL), dried over Na₂SO₄, and concentrated to give the crude product. The crude product was purified by preparative TLC (DCM:MeOH=10:1) to give (1S,2S)-2-(4-chloropyridin-2-yl)-N-(4-(((6-cyclopropylimidazo[1,2-a]pyridin-2-yl)methyl)amino)-6-oxo-1,6-dihydropyridin-2-yl)cyclopropane-1-carboxamide (3.7 mg, 5.4% yield) as a white solid. ESI-MS [M+H]+: 475.1. ¹H NMR (400 MHz, DMSO) δ 11.10-10.67 (m, 2H), 10.64-10.17 (m, 2H), 8.41 (d, J=5.3 Hz, 1H), 8.31 (s, 1H), 7.64 (d, J=6.4 Hz, 2H), 7.43-7.30 (m, 2H), 6.97 (dd, J=9.3, 1.7 Hz, 1H), 4.99 (s, 1H), 4.26 (d, J=5.5 Hz, 2H), 2.64-2.56 (m, 1H), 2.45-2.36 (m, 1H), 1.96-1.86 (m, 1H), 1.56-1.41 (m, 2H), 0.96-0.86 (m, 2H), 0.71-0.60 (m, 2H).

### Example 344

### Synthesis of (rac-(1S*,2S*))-2-(3-chlorophenyl)-N-(4-(((6-cyclopropylimidazo[1,2-a]pyridin-2-yl)methyl)amino)-1,3,5-triazin-2-yl)cyclopropane-1-carboxamide (I-344)

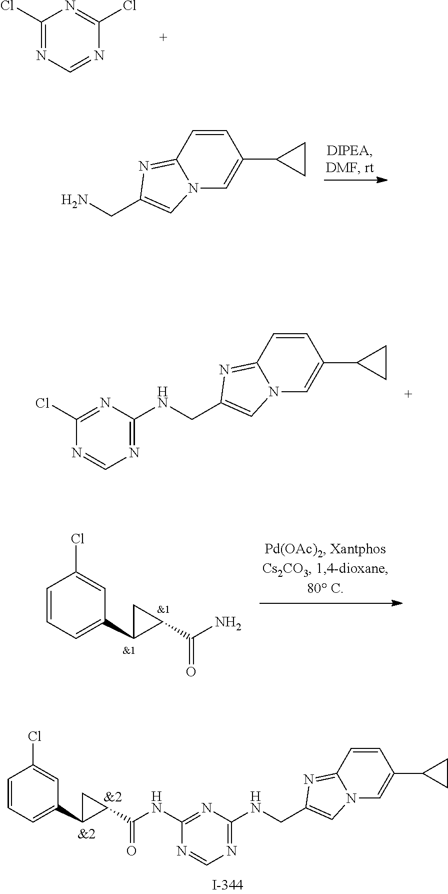 [SMILES: Clc1ncnc(Cl)n1 ; NCc1cn2cc(C3CC3)ccc2n1 ; Clc1ncnc(NCc2cn3cc(C4CC4)ccc3n2)n1 ; NC(=O)[C@H]1C[C@@H]1c1cccc(Cl)c1 ; O=C(Nc1ncnc(NCc2cn3cc(C4CC4)ccc3n2)n1)[C@H]1C[C@@H]1c1cccc(Cl)c1]

Synthesis of 4-chloro-N-((6-cyclopropylimidazo[1,2-a]pyridin-2-yl)methyl)-1,3,5-triazin-2-amine. DIPEA (0.52 mL, 3.0 mmol) was added to a stirred solution of 2,4-dichloro-1,3,5-triazine (150 mg, 1.0 mmol) and (6-cyclopropylimidazo[1,2-a]pyridin-2-yl)methanamine (190 mg, 1.0 mmol) in DMF (2.0 mL) and stirred at room temperature for 1 h. The reaction was diluted with water (25 mL) and brine (25 mL) and extracted with EtOAc (3×50 mL). The organics were combined, dried over MgSO₄, and concentrated in vacuo. The residue was purified by silica gel chromatography, eluting with a gradient of 0-5% MeOH in DCM to give the title compound (91 mg, 30%). ESI-MS (M+H)+: 301.1, 1H NMR: ¹H NMR (400 MHz, DMSO) δ 9.18-9.11 (m, 1H), 8.49 (s, 0.5H), 8.43 (s, 0.5H), 8.33-8.29 (m, 1H), 7.68 (s, 1H), 7.42-7.37 (m, 1H), 7.01-6.97 (m, 1H), 4.64-4.57 (m, 2H), 1.96-1.89 (m, 1H), 0.95-0.90 (m, 2H), 0.71-0.65 (m, 2H), 1:1 mixture of rotamers.

Synthesis of rac-(1S*,2S*))-2-(3-chlorophenyl)-N-(4-(((6-cyclopropylimidazo[1,2-a]pyridin-2-yl)methyl)amino)-1,3,5-triazin-2-yl)cyclopropane-1-carboxamide (I-344). 4-chloro-N-((6-cyclopropylimidazo[1,2-a]pyridin-2-yl)methyl)-1,3,5-triazin-2-amine (85 mg, 0.283 mmol), (rac-(1S*,2S*))-2-(3-chlorophenyl)cyclopropane-1-carboxamide (55 mg, 0.28 mmol), Xantphos (65 mg, 0.11 mmol), Cs₂CO₃ (140 mg, 0.42 mmol), and Pd(OAc)₂ (13 mg, 0.057 mmol) were suspended in 1,4-dioxane (2.0 mL) and degassed with N₂ for 5 min. The reaction was then heated to 80° C. under a N₂ environment for 1.5 h. The reaction was cooled to room temperature and diluted with water (25 mL) and brine (25 mL) and extracted with EtOAc (3×20 mL). The organics were combined, dried over MgSO₄ and concentrated in vacuo. The residue was purified by reverse phase preparative HPLC to give the title compound (13.7 mg, 11%). ESI-MS (M+H)+: 460.2, ¹H NMR (400 MHz, DMSO) δ 10.62 (s 0.4H), 10.61 (s, 0.6H), 8.40 (t, J=6.1 Hz, 0.6H), 8.37 (s, 0.4H), 8.30 (s, 0.6H), 8.29-8.26 (m, 1.4H), 7.65 (s, 0.6H), 7.60 (s, 0.4H), 7.36 (d, J=9.3 Hz, 1H), 7.32-7.20 (m, 3H), 7.18-7.14 (m, 0.4H), 7.12-7.08 (m, 0.6H), 6.96-6.93 (m, 1H), 4.58 (d, J=6.0 Hz, 0.8H), 4.52 (d, J=6.1 Hz, 1.2H), 2.78-2.75 (m, 0.6H), 2.64-2.61 (m, 0.4H), 2.47-2.39 (m, 1H), 1.95-1.87 (m, 1H), 1.52-1.45 (m, 1H), 1.43-1.37 (m, 1H), 0.94-0.88 (m, 2H), 0.69-0.64 (m, 2H), 6:4 mixture of rotamers.

### Example 345

### Synthesis of ((rac-(1S*,2S*))-2-(3-chlorophenyl)-N-(6-(((6-cyclopropylimidazo[1,2-a]pyridin-2-yl)methyl)amino)pyrazin-2-yl)cyclopropane-1-carboxamide (I-345)

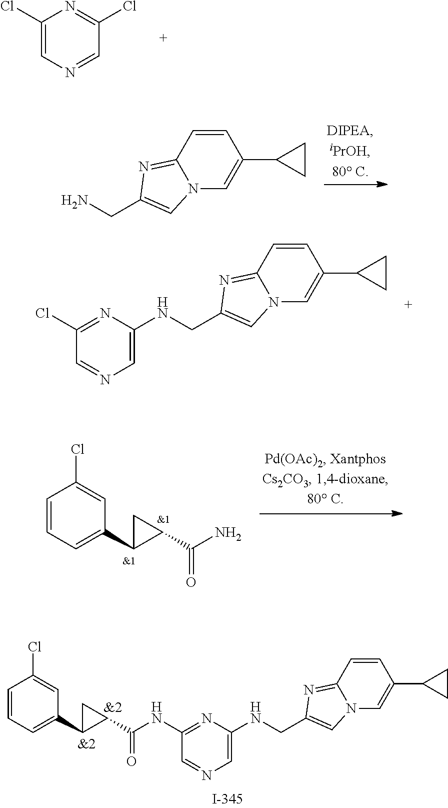 [SMILES: Clc1cncc(Cl)n1 ; NCc1cn2cc(C3CC3)ccc2n1 ; Clc1cncc(NCc2cn3cc(C4CC4)ccc3n2)n1 ; NC(=O)[C@H]1C[C@@H]1c1cccc(Cl)c1 ; O=C(Nc1cncc(NCc2cn3cc(C4CC4)ccc3n2)n1)[C@H]1C[C@@H]1c1cccc(Cl)c1]

Synthesis of 6-chloro-N-((6-cyclopropylimidazo[1,2-a]pyridin-2-yl)methyl)pyrazin-2-amine. DIPEA (0.26 mL, 1.51 mmol) was added to a stirred solution of 2,6-dichloropyrazine (75 mg, 0.50 mmol) and (6-cyclopropylimidazo[1,2-a]pyridin-2-yl)methanamine (100 mg, 0.55 mmol) in isopropanol (1.0 mL) and stirred at 80° C. for 18 h. The reaction was diluted with water (40 mL) and extracted with EtOAc (3×30 mL). The organics were combined, dried over MgSO₄, and concentrated in vacuo. The residue was purified by silica gel chromatography, eluting with a gradient of 0-5% MeOH in DCM to give the title compound (54 mg, 36%) as a formic acid salt. ESI-MS (M+H)+: 300.2

Synthesis of (rac-(1S*,2S*))-2-(3-chlorophenyl)-N-(6-(((6-cyclopropylimidazo[1,2-a]pyridin-2-yl)methyl)amino)pyrazin-2-yl)cyclopropane-1-carboxamide (I-345). 6-chloro-N-((6-cyclopropylimidazo[1,2-a]pyridin-2-yl)methyl)pyrazin-2-amine (50 mg, 0.17 mmol), 2 (rac-(1S*,2S*))-2-(3-chlorophenyl)cyclopropane-1-carboxamide (33 mg, 0.14 mmol), XantPhos (19 mg, 0.033 mmol), and Cs₂CO₃ (82 mg, 0.25 mmol), and Pd(OAc)₂ (15 mg, 0.067 mmol) were suspended in 1,4-dioxane (2.0 mL) and degassed with N₂ for 5 min. The reaction mixture was heated to 80° C. under a N₂ environment for 3 h. The reaction was cooled to room temperature and diluted with water (30 mL) and saturated brine solution (30 mL). The solution was extracted with DCM (3×20 mL) and the organics were combined, dried over MgSO₄ and concentrated in vacuo. The residue was purified by reverse phase preparative HPLC to give the title compound (6.7 mg, 5%). ESI-MS (M+H)+: 459.2, ¹H NMR (400 MHz, DMSO): δ, ppm 10.45 (1H, s), 8.46 (1H, s), 8.31 (1H, t, J=0.8 Hz), 7.74 (1H, s), 7.68 (1H, s), 7.50 (1H, t, J=5.8 Hz), 7.38 (1H, d, J=9.3 Hz), 7.35-7.30 (1H, m), 7.29-7.25 (2H, m), 7.17 (1H, td, J=1.3, 7.6 Hz), 6.97 (1H, dd, J=1.8, 9.3 Hz), 4.56 (2H, d, J=5.7 Hz), 2.48-2.39 (2H, m), 1.92 (1H, tdd, J=3.8, 9.3, 9.3 Hz), 1.50 (1H, ddd, J=4.1, 5.2, 9.2 Hz), 1.40 (1H, ddd, J=4.2, 6.4, 8.1 Hz), 0.92 (2H, ddd, J=4.3, 6.3, 8.4 Hz), 0.67 (2H, ddd, J=3.2, 4.6, 6.7 Hz).

### Example 346

### Synthesis of (rac-(1S*,2S*))-2-(3-chlorophenyl)-N-(4-(((6-cyclopropylimidazo[1,2-a]pyridin-2-yl)methyl)amino)pyrimidin-2-yl)cyclopropane-1-carboxamide (I-346)

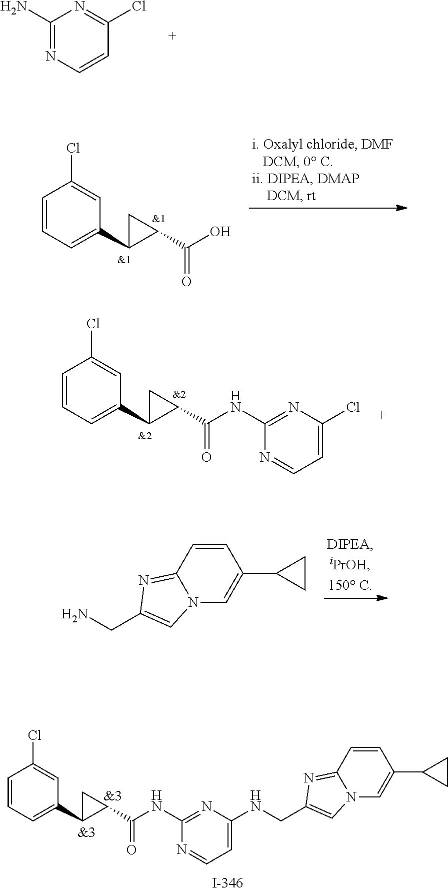 [SMILES: Nc1nccc(Cl)n1 ; O=C(O)[C@H]1C[C@@H]1c1cccc(Cl)c1 ; O=C(Nc1nccc(Cl)n1)[C@H]1C[C@@H]1c1cccc(Cl)c1 ; NCc1cn2cc(C3CC3)ccc2n1 ; O=C(Nc1nccc(NCc2cn3cc(C4CC4)ccc3n2)n1)[C@H]1C[C@@H]1c1cccc(Cl)c1]

Synthesis of (rac-(1S*,2S*))-2-(3-chlorophenyl)-N-(4-chloropyrimidin-2-yl)cyclopropane-1-carboxamide. Oxalyl chloride (0.33 mL, 3.81 mmol) was added to a stirred solution of (rac-(1S*,2S*))-2-(3-chlorophenyl)cyclopropane-1-carboxylic acid (250 mg, 1.27 mmol) and DMF (0.02 mL) in DCM (4 mL) and stirred at room temperature for 20 min. The reaction was concentrated in vacuo. The residue was dissolved in DCM (3 mL) and added to a stirred solution of 4-chloropyrimidin-2-amine (165 mg, 1.27 mmol) and DMAP (16 mg, 0.13 mmol) in DCM (1 mL). DIPEA (0.66 mL, 3.81 mmol) was then added and the reaction was stirred at room temperature for 18 h. The reaction was quenched with water (20 mL) and extracted with DCM (3×15 mL). The organics were combined, dried (MgSO₄), and concentrated in vacuo. The residue was purified by silica gel chromatography, eluting with a gradient of 0-100% EtOAc in cyclohexane to give the title compound (90 mg, 23%). 1H NMR: ¹H NMR (400 MHz, CDCl₃) δ 8.45 (d, J=5.3 Hz, 1H), 8.41 (s, 1H), 7.23-7.14 (m, 3H), 7.10-7.02 (m, 2H), 2.86-2.78 (m, 1H), 2.69-2.61 (m, 1H), 1.86-1.80 (m, 1H), 1.21-1.15 (m, 1H).

Synthesis of (rac-(1S*,2S*))-2-(3-chlorophenyl)-N-(4-(((6-cyclopropylimidazo[1,2-a]pyridin-2-yl)methyl)amino)pyrimidin-2-yl)cyclopropane-1-carboxamide (I-346). (rac-(1S*,2S*))-2-(3-chlorophenyl)-N-(4-chloropyrimidin-2-yl)cyclopropane-1-carboxamide (30 mg, 0.097 mmol), (6-cyclopropylimidazo[1,2-a]pyridin-2-yl)methanamine (22 mg, 0.12 mmol) and DIPEA (51 μL, 0.29 mmol) were dissolved in isopropanol (2.5 mL) and heated at 150° C. in the microwave for 1 h. The reaction was cooled to room temperature and concentrated in vacuo. The residue was purified by reverse phase preparative HPLC to give the title compound (3.1 mg, 7%) as a formic acid salt. ESI-MS (M+H)+: 459.3, ¹H NMR (400 MHz, DMSO) δ 10.22 (s, 1H), 8.35 (s, 1H), 8.29 (s, 1H), 7.94 (s, 1H), 7.86 (s, 1H), 7.73 (s, 1H), 7.43 (d, J=9.3 Hz, 1H), 7.33-7.30 (m, 3H), 7.17 (d, J=7.8 Hz, 1H), 7.01 (dd, J=1.4, 9.2 Hz, 1H), 6.30 (d, J=5.8 Hz, 1H), 4.59 (br s, 2H), 2.49-2.38 (m, 1H), 2.01-1.93 (m, 1H), 1.56-1.49 (m, 1H), 1.43-1.37 (m, 1H), 1.00-0.93 (m, 2H), 0.75-0.69 (m, 2H).

### Example 347

### Synthesis of (rac-(1S*,2S*))-2-(3-chlorophenyl)-N-(2-(((6-cyclopropylimidazo[1,2-a]pyridin-2-yl)methyl)amino)pyrimidin-4-yl)cyclopropane-1-carboxamide (I-347)

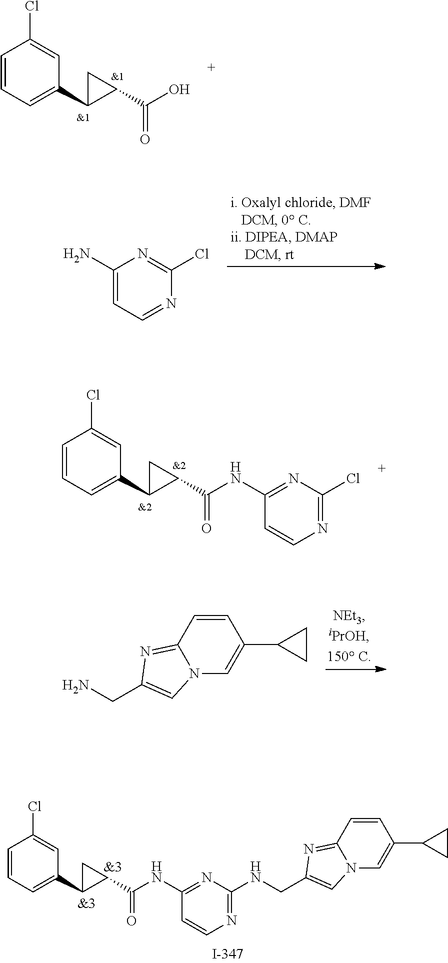 [SMILES: O=C(O)[C@H]1C[C@@H]1c1cccc(Cl)c1 ; Nc1ccnc(Cl)n1 ; O=C(Nc1ccnc(Cl)n1)[C@H]1C[C@@H]1c1cccc(Cl)c1 ; NCc1cn2cc(C3CC3)ccc2n1 ; O=C(Nc1ccnc(NCc2cn3cc(C4CC4)ccc3n2)n1)[C@H]1C[C@@H]1c1cccc(Cl)c1]

Synthesis of (rac-(1S*,2S*))-2-(3-chlorophenyl)-N-(2-chloropyrimidin-4-yl)cyclopropane-1-carboxamide. Oxalyl chloride (0.27 mL, 3.05 mmol) was added to a stirred solution of (rac-(1S*,2S*))-2-(3-chlorophenyl)cyclopropane-1-carboxylic acid (200 mg, 1.02 mmol) and DMF (0.02 mL) in DCM (4 mL) and stirred at room temperature for 20 min. The reaction was concentrated in vacuo. The residue was dissolved in DCM (3.0 mL) and added to a stirred solution of 2-chloropyrimidin-4-amine (130 mg, 1.02 mmol) and DMAP (25 mg, 0.20 mmol) in DCM (1.0 mL). DIPEA (0.27 mL, 1.53 mmol) was then added and the reaction was stirred at rt for 18 h. The reaction was quenched with water (20 mL) and extracted with DCM (3×15 mL). The organics were combined, dried over MgSO₄, and concentrated in vacuo. The residue was purified by silica gel chromatography, eluting with a gradient of 0-100% EtOAc in cyclohexane to give the title compound (145 mg, 46%). ESI-MS (M+H)+: 308.1, ¹H NMR (400 MHz, DMSO) δ 11.62 (s, 1H), 8.60 (d, J=5.8 Hz, 1H), 8.07 (d, J=5.8 Hz, 1H), 7.34-7.28 (m, 3H), 7.18 (d, J=7.5 Hz, 1H), 2.41-2.35 (m, 1H), 1.59-1.50 (m, 2H), one proton hidden under DMSO peak.

Synthesis of (rac-(1S*,2S*))-2-(3-chlorophenyl)-N-(2-(((6-cyclopropylimidazo[1,2-a]pyridin-2-yl)methyl)amino)pyrimidin-4-yl)cyclopropane-1-carboxamide (I-347). (rac-(1S*,2S*))-2-(3-chlorophenyl)-N-(2-chloropyrimidin-4-yl)cyclopropane-1-carboxamide (75 mg, 0.24 mmol), (6-cyclopropylimidazo[1,2-a]pyridin-2-yl)methanamine (46 mg, 0.24 mmol), and triethylamine (0.1 mL, 0.73 mmol) were dissolved in isopropanol (2.5 mL) and heated at 150° C. in the microwave for 30 min. The reaction was cooled to room temperature and concentrated in vacuo. The residue was purified by reverse phase preparative HPLC to give the title compound (23 mg, 21%) as a formic acid salt. ESI-MS (M+H)+: 459.3, ¹H NMR (400 MHz, DMSO) δ 10.61 (s, 1H), 8.28 (s, 1H), 8.16 (d, J=5.7 Hz, 1H), 7.58 (s, 1H), 7.37-7.29 (m, 2H), 7.28-7.25 (m, 3H), 7.15 (d, J=7.7 Hz, 1H), 6.93 (dd, J=1.8, 9.3 Hz, 1H), 4.56 (d, J=6.0 Hz, 2H), 2.47-2.38 (m, 2H), 1.93-1.85 (m, 1H), 1.52-1.37 (m, 2H), 0.92-0.87 (m, 2H), 0.67-0.62 (m, 2H).

### Example 348

### Synthesis of (rac-(1S*,2S*))-2-(3-chlorophenyl)-N-(6-(((6-cyclopropylimidazo[1,2-a]pyridin-2-yl)methyl)amino)pyridazin-4-yl)cyclopropane-1-carboxamide (I-348)

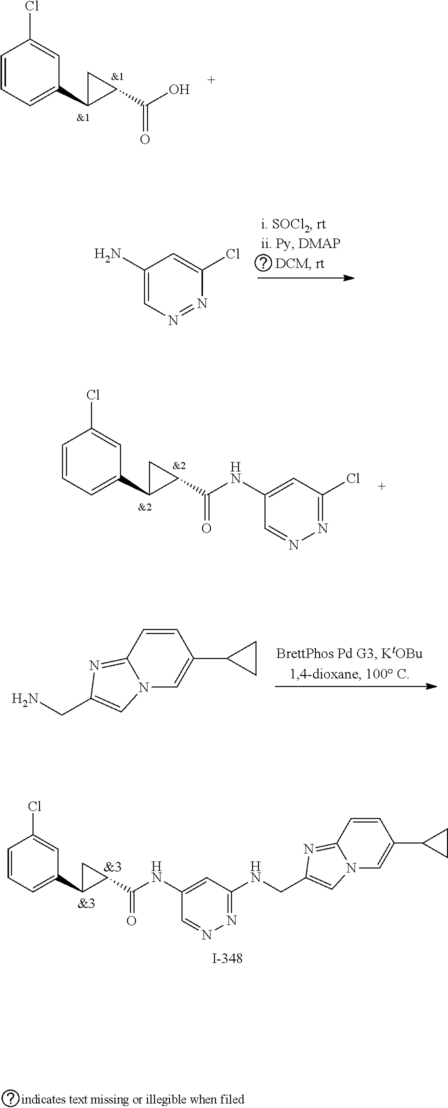 [SMILES: O=C(O)[C@H]1C[C@@H]1c1cccc(Cl)c1 ; Nc1cnnc(Cl)c1 ; O=C(Nc1cnnc(Cl)c1)[C@H]1C[C@@H]1c1cccc(Cl)c1 ; NCc1cn2cc(C3CC3)ccc2n1 ; O=C(Nc1cnnc(NCc2cn3cc(C4CC4)ccc3n2)c1)[C@H]1C[C@@H]1c1cccc(Cl)c1]

Synthesis of (rac-(1S*,2S*))-2-(3-chlorophenyl)-N-(6-chloropyridazin-4-yl)cyclopropane-1-carboxamide. rac-(1S*,2S*)-2-(3-Chlorophenyl)cyclopropane-1-carboxylic acid (180 mg, 0.91 mmol) was dissolved in thionyl chloride (0.33 mL, 4.57 mmol) and stirred at rt for 40 min. The reaction was concentrated in vacuo. The residue was dissolved in DCM (5.0 mL) and added to a stirred solution of 6-chloropyridazin-4-amine (118 mg, 0.913 mmol) in DCM (5.0 mL). Pyridine (0.11 mL, 1.37 mmol) was then added and the reaction was stirred at room temperature for 18 h. The reaction was quenched with water (20 mL) and extracted with DCM (3×15 mL), the organics were combined, dried (MgSO₄), and concentrated in vacuo. The residue was purified by silica gel chromatography, eluting with a gradient of 0-10% MeOH in DCM to give the title compound (100 mg, 36%). ESI-MS (M+H)+: 309.1, ¹H NMR (400 MHz, CDCl₃) δ 9.05 (d, J=2.3 Hz, 1H), 8.95 (s, 1H), 8.34 (d, J=2.1 Hz, 1H), 7.25-7.18 (m, 2H), 7.08 (s, 1H), 7.05-7.02 (m, 1H), 2.65-2.58 (m, 1H), 2.14-2.08 (m, 1H), 1.82-1.76 (m, 1H), 1.50-1.43 (m, 1H).

Synthesis of (rac-(1S*,2S*))-2-(3-chlorophenyl)-N-(6-(((6-cyclopropylimidazo[1,2-a]pyridin-2-yl)methyl)amino)pyridazin-4-yl)cyclopropane-1-carboxamide (I-348). rac-(1S*,2S*)-2-(3-Chlorophenyl)-N-(6-chloropyridazin-4-yl)cyclopropane-1-carboxamide (69 mg, 0.22 mmol), (6-cyclopropylimidazo[1,2-a]pyridin-2-yl)methanamine (62 mg, 0.33 mmol), and K^{t}OBu (75 mg, 0.67 mmol) were suspended in 1,4-dioxane (2.7 mL) and degassed with N₂ for 5 min, BrettPhos Pd G3 (20 mg, 0.022 mmol) was then added and the reaction was heated at 100° C. under a N₂ environment for 18 h. The reaction was cooled to room temperature and filtered through Celite©. The filtrate was diluted with EtOAc (40 mL) and water (40 mL). The layers were separated and the aqueous layer was then further extracted with EtOAc (2×20 mL). The organics were combined, dried (MgSO₄), and concentrated in vacuo. The residue was purified by reverse phase preparative HPLC to give the title compound (8.4 mg, 8%). ESI-MS (M+H)+: 459.3, 1H NMR: ¹H NMR (400 MHz, DMSO) δ 10.59 (s, 1H), 8.49 (d, J=2.1 Hz, 1H), 8.31 (s, 1H), 7.65 (s, 1H), 7.41-7.26 (m, 6H), 7.20 (d, J=7.5 Hz, 1H), 6.97 (dd, J=1.8, 9.3 Hz, 1H), 4.61 (d, J=5.6 Hz, 2H), 2.48-2.41 (m, 1H), 2.17-2.11 (m, 1H), 1.96-1.88 (m, 1H), 1.58-1.46 (m, 2H), 0.95-0.89 (m, 2H), 0.70-0.65 (m, 2H).

### Example 349

### Synthesis of 2-(3-chlorophenyl)-2-cyano-N-(6-(((6-cyclopropylimidazo[1,2-a]pyridin-2-yl)methyl)amino)pyrimidin-4-yl)cyclopropane-1-carboxamide (I-349)

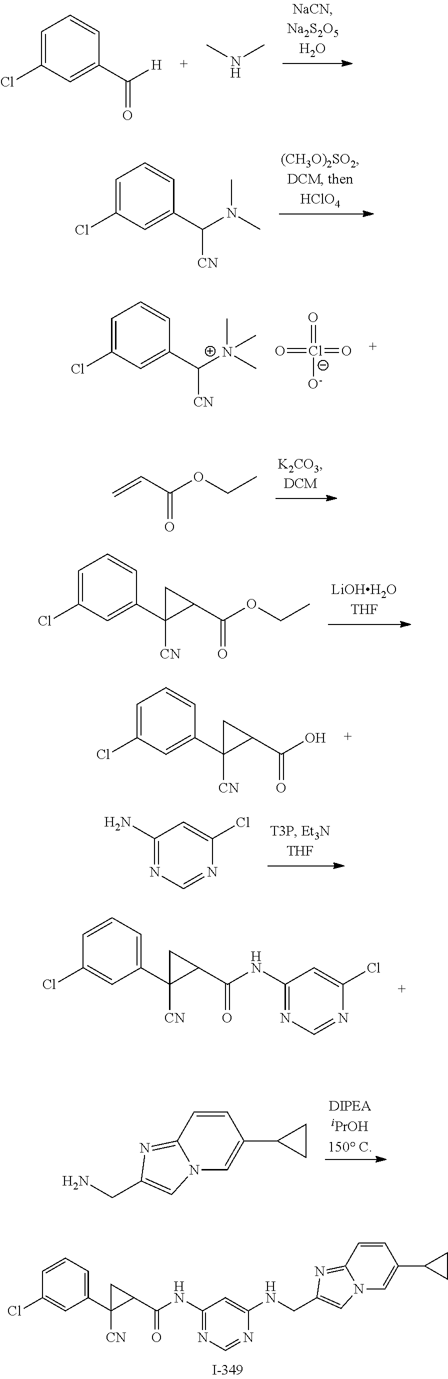

Synthesis of 2-(3-chlorophenyl)-2-(dimethylamino)acetonitrile. 3-chlorobenzaldehyde (2.0 g, 14.23 mmol) was added dropwise to a solution of Na₂S₂O₅ (2.7 g, 14.23 mmol) in water (7 mL), followed by dimethylamine (40% aq·, 1.8 mL, 14.23 mmol) at room temperature. After 30 min the mixture was cooled to 0° C. and a solution of NaCN (0.7 g, 14.23 mmol) in water (3 mL) was added. The mixture was allowed to warm to room temperature and stirred for 18 h. The reaction mixture was diluted with water and extracted with DCM (3×10 mL) The organic phases were combined and passed through a phase separator cartridge and concentrated in vacuo to give the title compound as a residue that was taken on to the next stage without further purification.

Synthesis of 1-(3-chlorophenyl)-1-cyano-N,N,N-trimethylmethammonium perchlorate. Dimethyl sulfate (1.60 mL, 14.23 mmol) was added to a cooled solution of 2-(3-chlorophenyl)-2-(dimethylamino)acetonitrile (2.77 g, 14.23 mmol) in DCM (10 mL) at 0° C. After 2 h the mixture was allowed to warm to room temperature and stirred for 18 h. The mixture was concentrated in vacuo and the residue was dissolved in water (10 mL) then extracted with DCM (3×10 mL). The aqueous phase was treated with HClO₄ (70%, aq·0.9 mL). The precipitate formed was collected, washed with Et₂O, dried to give the title compound (1.37 g, 31% as a perchlorate salt).

Synthesis of Ethyl 2-(3-chlorophenyl)-2-cyanocyclopropane-1-carboxylate. Potassium carbonate (2.24 g, 16.17 mmol) was added to a vigorously stirred mixture of 1-(3-chlorophenyl)-1-cyano-N,N,N-trimethylmethammonium perchlorate (0.50 g, 1.52 mmol) followed by a solution of ethyl acrylate (0.88 mL, 8.09 mmol) in DCM (20 mL). The mixture was stirred at room temperature for 18 h. The mixture was diluted with water (20 mL) and extracted with DCM (3×20 mL). The organic phases were combined and passed through a phase separator cartridge and concentrated in vacuo. Purification by silica gel column chromatography, eluting with a gradient of 0-40% ethyl acetate in hexane to give the title compound (0.17 g, 43%) as a clear oil (mixture of E:Z isomers). ESI-MS: (M+H)+: 250.1, Isomer 1: ¹H NMR (400 MHz, CDCl₃) δ 7.39-7.27 (m, 4H), 4.30 (q, J=7.0 Hz, 2H), 2.37 (dd, J=7.7, 7.7 Hz, 1H), 2.30-2.26 (m, 1H), 1.84 (dd, J=5.8, 8.3 Hz, 1H), 1.34 (dd, J=7.1, 7.1 Hz, 3H). Isomer 2: ¹H NMR (400 MHz, CDCl₃) δ 7.39-7.27 (m, 4H), 3.99-3.91 (m, 2H), 2.73 (dd, J=6.9, 8.7 Hz, 1H), 2.23-2.17 (m, 1H), 1.95 (dd, J=5.8, 8.6 Hz, 1H), 1.05 (dd, J=7.1, 7.1 Hz, 3H).

Synthesis of 2-(3-chlorophenyl)-2-cyanocyclopropane-1-carboxylic acid. Lithium hydroxide (1M, aq, 0.99 mL, 0.99 mmol) was added dropwise to a cooled solution of ethyl 2-(3-chlorophenyl)-2-cyanocyclopropane-1-carboxylate (124 mg, 0.50 mmol) at 0° C. in THF (5 mL), and the resulting mixture was stirred for 2 h. The mixture was diluted with water, acidified with HCl (1 M. aq, 2 mL), and extracted with EtOAc (3×10 mL). The organic phases were combined, dried over MgSO₄, filtered, and concentrated in vacuo to give the title compound as a clear oil (105 mg, 95%) which was used without further purification. Isomer 1: ¹H NMR (400 MHz, CDCl₃) δ 7.45-7.28 (m, 4H), 2.38 (dd, J=7.7, 7.7 Hz, 1H), 2.28-2.24 (m, 1H), 1.95-1.85 (m, 1H). Isomer 2: ¹H NMR (400 MHz, CDCl₃) δ 7.45-7.28 (m, 4H), 2.79-2.73 (m, 1H), 2.22-2.17 (m, 1H), 2.05-2.01 (m, 1H).

Synthesis of 2-(3-chlorophenyl)-N-(6-chloropyrimidin-4-yl)-2-cyanocyclopropane-1-carboxamide. T3P (1.53 mL, 2.58 mmol) was added dropwise to a solution of 2-(3-chlorophenyl)-2-cyanocyclopropane-1-carboxylic acid (228 mg, 1.03 mmol), 4-amino-6-chloro-pyrimidine (120 mg, 0.93 mmol) and triethylamine (0.50 mL, 3.61 mmol) in THF (5 mL). The mixture was stirred for 60 h, then concentrated in vacuo. The residue was partitioned between EtOAc (10 mL) and NaHCO₃ solution (sat·aq·, 10 mL). The aqueous layer was extracted with EtOAc (2×10 mL). The combined organics were dried over MgSO₄ then concentrated in vacuo. The residue was purified by silica gel column chromatography, eluting with a gradient of 0-50% ethyl acetate in cyclohexane to give the title compound (88 mg, 26%) as a pink solid which was used without further purification.

Synthesis of 2-(3-chlorophenyl)-2-cyano-N-(6-(((6-cyclopropylimidazo[1,2-a]pyridin-2-yl)methyl)amino)pyrimidin-4-yl)cyclopropane-1-carboxamide (I-349). Using similar procedure to that for rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide, the title compound was made from 2-(3-chlorophenyl)-N-(6-chloropyrimidin-4-yl)-2-cyanocyclopropane-1-carboxamide and (6-cyclopropylimidazo[1,2-a]pyridin-2-yl)methanamine hydrochloride (5 mg, 7%). ESI-MS (M+H): 484.4, ¹H NMR (400 MHz, DMSO) δ 10.99-10.94 (m, 1H), 8.31 (s, 1H), 8.25 (s, 1H), 7.83-7.82 (m, 1H), 7.63-7.59 (m, 1H), 7.56-7.49 (m, 1H), 7.45-7.36 (m, 3H), 7.01-6.97 (m, 2H), 6.78-6.72 (m, 1H), 4.64-4.50 (m, 2H), 2.32-2.28 (m, 1H), 2.15-2.09 (m, 1H), 1.99-1.91 (m, 1H), 0.99-0.92 (m, 2H), 0.73-0.67 (m, 2H). 1H missing, assumed under water peak

### Example 350

### Synthesis of (1S,2S)-2-(3-chlorophenyl)-N-(6-(((6-cyclopropylimidazo[1,2-a]pyridin-2-ylmethyl amino)pyrimidin-4-yl)-N-methylcyclopropane-1-carboxamide

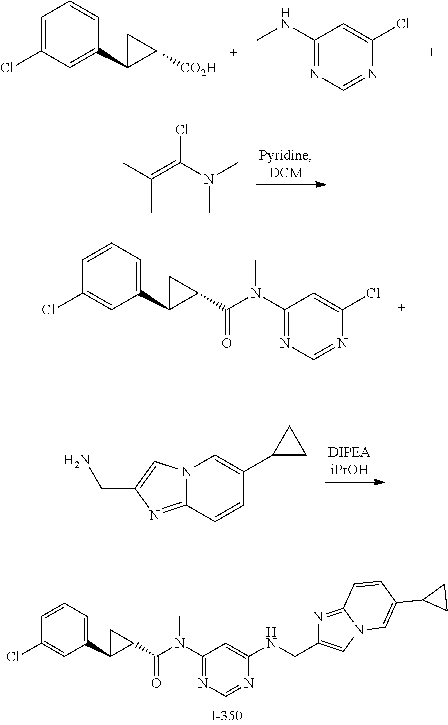

Synthesis of (1S,2S)-2-(3-chlorophenyl)-N-(6-chloropyrimidin-4-yl)-N-methylcyclopropane-1-carboxamide. 1-chloro-N,N,2-trimethylprop-1-en-1-amine (0.081 mL, 0.61 mmol) was added to a N₂ purged solution of (1S,2S)-2-(3-chlorophenyl)cyclopropane-1-carboxylic acid (100 mg, 0.509 mmol) in DCM (2.0 mL) and the mixture was stirred at room temperature. After 2 h, 6-chloro-N-methylpyrimidin-4-amine (80 mg, 0.559 mmol) was added, followed by pyridine (0.062 mL, 0.763 mmol), and the mixture was stirred at room temperature for 18 h. The mixture was diluted with water (5.0 mL), then extracted with DCM (3×5 mL). The organics were concentrated in vacuo onto silica gel and purified by silica gel column chromatography, eluting with a gradient of 0-100% ethyl acetate in hexane to give the title compound (75 mg, 45%). ESI-MS (M+H)+: 322.1, ¹H NMR (400 MHz, CDCl₃) δ 8.74 (d, J=0.8 Hz, 1H), 7.81 (s, 1H), 7.25-7.20 (m, 2H), 7.12 (d, J=2.0 Hz, 1H), 7.05-7.01 (m, 1H), 3.60 (s, 3H), 2.62 (ddd, J=4.2, 6.4, 9.1 Hz, 1H), 2.23 (ddd, J=4.2, 5.4, 8.1 Hz, 1H), 1.88-1.82 (m, 1H), 1.49-1.42 (m, 1H).

Synthesis of (1S,2S)-2-(3-chlorophenyl)-N-(6-(((6-cyclopropylimidazo[1,2-a]pyridin-2-yl)methyl)amino)pyrimidin-4-yl)-N-methylcyclopropane-1-carboxamide (I-350). Using similar procedure to that for rac-(1S*,2S*)-2-(3-chlorophenyl)-N-(6-(((6-cyclopropylimidazo[1,2-a]pyridin-2-yl)methyl)amino)pyrimidin-4-yl)cyclopropane-1-carboxamide, the title compound was made from (1S,2S)-2-(3-chlorophenyl)-N-(6-chloropyrimidin-4-yl)-N-methylcyclopropane-1-carboxamide and (6-cyclopropylimidazo[1,2-a]pyridin-2-yl)methanamine (25 mg, 23%). ESI-MS (M+H): 473.5, ¹H NMR (400 MHz, DMSO) δ 8.35-8.34 (m, 2H), 7.93 (t, J=5.8 Hz, 1H), 7.69 (s, 1H), 7.42 (d, J=9.3 Hz, 1H), 7.32-7.25 (m, 4H), 7.01 (dd, J=1.8, 9.3 Hz, 1H), 6.65-6.61 (m, 1H), 4.66-4.59 (m, 2H), 2.53-2.49 (m, 1H), 2.32 (s, 1H), 2.00-1.92 (m, 1H), 1.59-1.53 (m, 1H), 1.34-1.28 (m, 1H), 0.99-0.93 (m, 2H), 0.74-0.68 (m, 2H). 3H missing, assumed under DMSO or water peak.

### Example 351

### Synthesis of rac-(1S*,2S*)—N-(6-(((6-cyclopropylimidazo[1,2-a]pyridin-2-yl)methyl)amino)pyrimidin-4-yl)-2-(3-cyclopropylphenyl)cyclopropane-1-carboxamide (I-351)

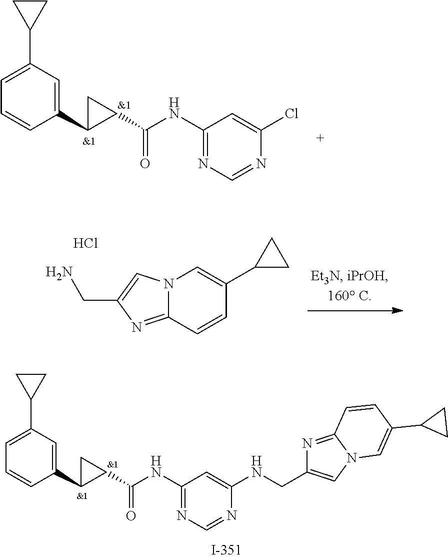

To a suspension of (6-cyclopropylimidazo[1,2-a]pyridin-2-yl)methanamine hydrochloride (140 mg, 0.45 mmol) and rac-(1S*,2S*)—N-(6-chloropyrimidin-4-yl)-2-(3-cyclopropylphenyl)cyclopropane-1-carboxamide (130 mg, 0.58 mmol) in 2-propanol (4.0 mL) was added triethylamine (0.25 mL, 1.8 mmol) and the reaction mixture was stirred at 80° C. until dissolution, then stirred in a microwave at 160° C. for 4.5 h. The reaction mixture was allowed to cool to room temperature and concentrated in vacuo. The residue was dissolved in DCM (25 mL) and washed with water (2×25 mL). The organics were passed through a hydrophobic frit and concentrated in vacuo. The residue was purified by preparative HPLC to give the title compound (47 mg, 22%) as an off white solid. ESI-MS (M+H): 465.4, ¹H NMR (400 MHz, DMSO) δ 10.62 (s, 1H), 8.35 (s, 1H), 8.24 (s, 1H), 7.89-7.89 (m, 1H), 7.66 (s, 1H), 7.42 (d, J=9.3 Hz, 1H), 7.35-7.30 (m, 1H), 7.23-7.17 (m, 1H), 7.04-6.98 (m, 1H), 6.98-6.93 (m, 2H), 6.92-6.89 (m, 1H), 4.62-4.61 (m, 2H), 2.41-2.35 (m, 2H), 2.01-1.88 (m, 2H), 1.52-1.45 (m, 1H), 1.42-1.35 (m, 1H), 1.01-0.92 (m, 4H), 0.74-0.67 (m, 4H).

### Example 352

### Synthesis of rac-(1S*,2S*)-2-(5-chloro-2-cyanophenyl)-N-(6-((6-cyclopropylimidazo[1,2-a]pyridin-2-yl)(hydroxy)methyl)pyrimidin-4-yl)cyclopropane-1-carboxamide (I-352)

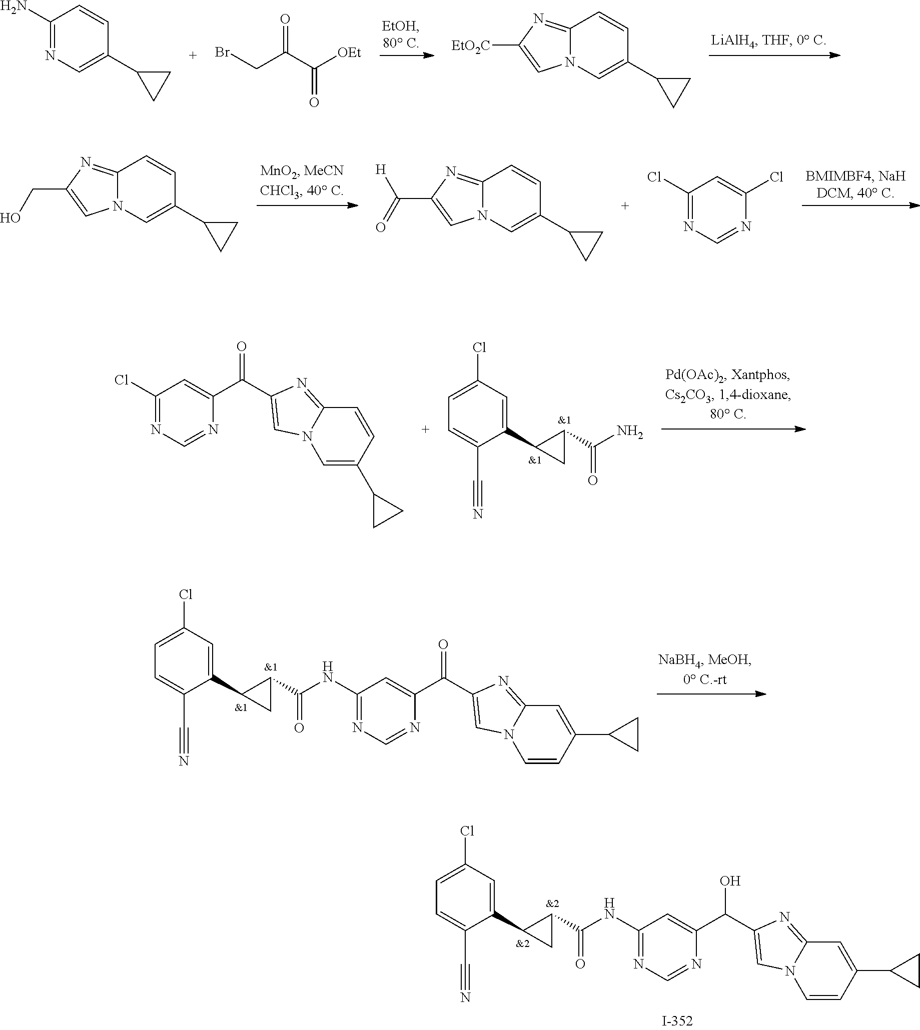

Synthesis of ethyl 6-cyclopropylimidazo[1,2-a]pyridine-2-carboxylate. 5-Cyclopropylpyridin-2-amine (1.5 g, 11 mmol) and ethyl bromopyruvate (1.7 mL, 13 mmol) were dissolved in EtOH (164 mL) and heated to 80° C. for 1 h. The reaction was cooled to room temperature and concentrated in vacuo. The residue was diluted with DCM (100 mL) and washed with NaHCO₃ (sat·aq·, 3×50 mL). The combined organics were dried over MgSO₄, then concentrated in vacuo. The residue was purified by column chromatography on silica gel, eluting with 0-100% EtOAc in cyclohexane to give the title compound (1.76 g, 68%). ESI-MS (M+H)+: 231, ¹H NMR (400 MHz, CDCl₃) δ 8.09 (s, 1H), 7.90 (s, 1H), 7.57 (d, J=9.5 Hz, 1H), 6.99 (dd, J=1.8, 9.4 Hz, 1H), 4.45 (q, J=7.2 Hz, 2H), 1.94-1.86 (m, 1H), 1.44 (t, J=7.0 Hz, 3H), 1.03-0.97 (m, 2H), 0.73-0.68 (m, 2H).

Synthesis of (6-cyclopropylimidazo[1,2-a]pyridin-2-yl)methanol. LiAlH₄ (1.0 M in THF, 17 mL, 17 mmol) was added dropwise to a stirred solution of ethyl 6-cyclopropylimidazo[1,2-a]pyridine-2-carboxylate (2.0 g, 8.7 mmol) in THF (25 mL) at 0° C. The reaction was stirred at 0° C. for 1 h. The reaction was quenched by pouring into NH₄Cl (sat·aq·, 50 mL). The mixture was diluted with DCM, filtered through Celite®, and washed with DCM. The layers were separated and the aqueous phase was further extracted with DCM (2×20 mL). The combined organics were dried over MgSO₄, then concentrated in vacuo to give the title compound (1.3 g, 80%) which was carried forward without further purification. ESI-MS (M+H)+: 189, ¹H NMR (400 MHz, CDCl₃) δ 7.87 (d, J=0.4 Hz, 1H), 7.44 (d, J=8.0 Hz, 2H), 6.93 (dd, J=1.8, 9.4 Hz, 1H), 4.82 (s, 2H), 1.92-1.84 (m, 1H), 0.99-0.93 (m, 2H), 0.70-0.65 (m, 2H).

Synthesis of 6-cyclopropylimidazo[1,2-a]pyridine-2-carbaldehyde. MnO₂ (3.1 g, 36 mmol) was added to a stirred solution of (6-cyclopropylimidazo[1,2-a]pyridin-2-yl)methanol (670 mg, 3.6 mmol) in MeCN (10 mL) and CHCl₃ (10 mL). The reaction was heated at 40° C. for 1 h. The reaction was cooled to room temperature and filtered through Celite®. The Celite® was washed with DCM and the filtrate was concentrated in vacuo to give the title compound (520 mg, 78%) which was carried forward without further purification. ESI-MS (M+H)+: 187, ¹H NMR (400 MHz, CDCl₃) δ 10.13 (s, 1H), 8.07 (s, 1H), 7.93 (d, J=0.4 Hz, 1H), 7.58 (d, J=9.5 Hz, 1H), 7.03 (dd, J=1.7, 9.5 Hz, 1H), 1.95-1.87 (m, 1H), 1.05-0.99 (m, 2H), 0.75-0.70 (m, 2H).

Synthesis of (6-chloropyrimidin-4-yl)(6-cyclopropylimidazo[1,2-a]pyridin-2-yl)methanone. 1-Butyl-3-methylimidazolium tetrafluoroborate (0.17 mL, 0.91 mmol) was added to a stirred degassed solution of 4,6-dichloropyrimidine (320 mg, 2.1 mmol) and 6-cyclopropylimidazo[1,2-a]pyridine-2-carbaldehyde (520 mg, 2.8 mmol) in DCM (50 mL), followed by the addition of NaH (60% in mineral oil, 110 mg, 2.8 mmol). The reaction was then heated at 40° C. for 1 h and cooled to room temperature. The reaction was diluted with DCM (30 mL) and washed with water (2×30 mL) and brine (sat·aq·, 30 mL). The combined organics were dried over MgSO₄ then concentrated in vacuo. The residue was purified by column chromatography on silica gel, eluting with 0-100% EtOAc in cyclohexane to give the title compound (320 mg, 50%). ESI-MS (M+H)+: 299, ¹H NMR (400 MHz, CDCl₃) δ 9.20 (s, 1H), 8.88 (s, 1H), 8.24 (s, 1H), 7.96 (d, J=0.4 Hz, 1H), 7.61 (d, J=9.4 Hz, 1H), 7.04 (dd, J=1.8, 9.5 Hz, 1H), 1.96-1.88 (m, 1H), 1.06-1.00 (m, 2H), 0.76-0.71 (m, 2H).

Synthesis of rac-(1S*,2S*)-2-(5-chloro-2-cyanophenyl)-N-(6-(6-cyclopropylimidazo[1,2-a]pyridine-2-carbonyl)pyrimidin-4-yl)cyclopropane-1-carboxamide. A suspension of (6-chloropyrimidin-4-yl)(6-cyclopropylimidazo[1,2-a]pyridin-2-yl)methanone (41 mg, 0.14 mmol), rac-(1S*,2S*)-2-(5-chloro-2-cyanophenyl)cyclopropane-1-carboxamide (3 mg, 0.14 mmol), XantPhos (15 mg, 0.03 mmol), and Cs₂CO₃ (66 mg, 0.20 mmol) in 1,4-dioxane (2.0 mL) was degassed for 5 min. Pd(OAc)₂ (6.1 mg, 0.03 mmol) was added and the reaction mixture was stirred at 80° C. for 16 h. The reaction mixture was cooled to room temperature, filtered through Celite®, and concentrated in vacuo. The residue was purified by column chromatography on silica gel, eluting with a gradient of 0-10% MeOH in DCM to give of crude title compound (21 mg) which was used in the next step without further purification.

Synthesis of rac-(1S*,2S*)-2-(5-chloro-2-cyanophenyl)-N-(6-((6-cyclopropylimidazo[1,2-a]pyridin-2-yl)(hydroxy)methyl)pyrimidin-4-yl)cyclopropane-1-carboxamide (I-352). NaBH₄ (1.8 mg, 0.05 mmol) was added portion-wise to a stirred solution of rac-(1S*,2S*)-2-(5-chloro-2-cyanophenyl)-N-(6-(6-cyclopropylimidazo[1,2-a]pyridine-2-carbonyl)pyrimidin-4-yl)cyclopropane-1-carboxamide (21 mg, 0.04 mmol) in MeOH (2.0 mL) at 0° C. and stirred at 0° C. for 5 min. The reaction was then allowed to warm to room temperature and stirred for 30 min. The reaction was quenched with NH₄Cl (sat·aq·, 10 mL) and extracted with EtOAc (3×15 mL). The combined organics were dried over MgSO₄ then concentrated in vacuo. The residue was purified by preparative HPLC to give of the title compound (5 mg, 23%). ESI-MS (M+H+): 485.3, ¹H NMR (400 MHz, DMSO) δ 11.24 (s, 1H), 8.76 (d, J=1.0 Hz, 1H), 8.38 (dd, J=1.8, 1.8 Hz, 1H), 8.33 (dd, J=0.8, 0.8 Hz, 1H), 7.89 (dd, J=1.0, 8.3 Hz, 1H), 7.72 (d, J=2.3 Hz, 1H), 7.55 (ddd, J=0.8, 2.0, 8.3 Hz, 1H), 7.46 (s, 1H), 7.36 (dd, J=2.3, 9.4 Hz, 1H), 6.97 (ddd, J=1.8, 1.8, 9.4 Hz, 1H), 6.16 (dd, J=3.5, 4.8 Hz, 1H), 5.71 (d, J=4.8 Hz, 1H), 2.67-2.64 (m, 1H), 1.97-1.89 (m, 1H), 1.71-1.66 (m, 1H), 1.65-1.60 (m, 1H), 0.95-0.90 (m, 2H), 0.70-0.65 (m, 2H) (one CH of cyclopropyl obscured by DMSO).

### Synthesis of rac-(1S*,2S*)—N-(6-chloro-2-(fluoromethyl)pyrimidin-4-yl)-2-(4-methylpyrimidin-2-yl)cyclopropane-1-carboxamide

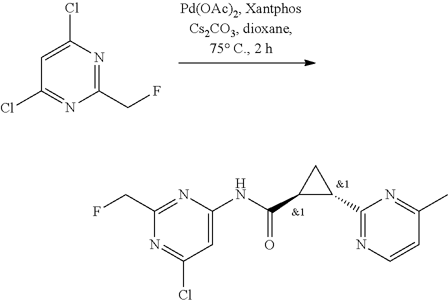 [SMILES: FCc1nc(Cl)cc(Cl)n1 ; Cc1ccnc([C@H]2C[C@@H]2C(=O)Nc2cc(Cl)nc(CF)n2)n1]

To a solution of 4,6-dichloro-2-(fluoromethyl)pyrimidine (50 mg, 0.28 mmol) in dioxane (3 mL) were added rac-(1S*,2S*)-2-(4-methylpyrimidin-2-yl)cyclopropane-1-carboxamide (50 mg, 0.28 mmol), Pd₂(dba)₃ (26 mg, 0.028 mmol), Xantphos (16 mg, 0.028 mmol), and Cs₂CO₃ (274 mg, 0.84 mmol). Then the mixture was stirred for 2 h at 75° C. under N₂. Water (10 mL) was added and extracted with EtOAc (3×10 mL). The organic layers were concentrated to give the crude, which was purified by preparative TLC (DCM/MeOH=20/1) to give rac-(1S*,2S*)—N-(6-chloro-2-(fluoromethyl)pyrimidin-4-yl)-2-(4-methylpyrimidin-2-yl)cyclopropane-1-carboxamide as a white solid (20 mg, 15%). ESI-MS [M+H]⁺: 322.1.

The compounds in Table 32 were prepared in a similar manner to rac-(1S*,2S*)—N-(6-chloro-2-(fluoromethyl)pyrimidin-4-yl)-2-(4-methylpyrimidin-2-yl)cyclopropane-1-carboxamide from appropriately substituted cyclopropyl amide and a di-halo-pyrimidine.

TABLE 32

| Compound | Analytical Data |
|---|---|
| 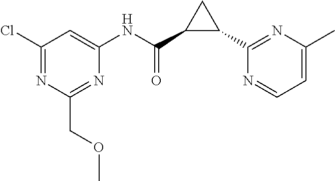 [SMILES: COCc1nc(Cl)cc(NC(=O)[C@H]2C[C@@H]2c2nccc(C)n2)n1] | ESI-MS [M + H]+: 334.2 |
| (1S,2S)-N-(6-chloro-2-(methoxymethyl)pyrimidin-4- | |
| yl)-2-(4-methylpyrimidin-2-yl)cyclopropane-1- | |
| carboxamide | |
| 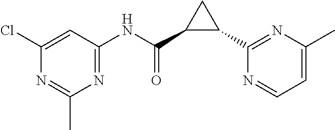 [SMILES: Cc1ccnc([C@H]2C[C@@H]2C(=O)Nc2cc(Cl)nc(C)n2)n1] | ESI-MS [M + H]+: 304.2. ¹H NMR (400 MHz, DMSO) δ 11.57 (s, 1H), 8.54 (d, J = 5.1 Hz, 1H), 7.95 (s, 1H), 7.22 (d, J = 5.1 Hz, 1H), 2.69 - 2.65 (m, 1H), 2.62- 2.57 (m, 1H), 2.51 (s, 3H), 2.42 (s, 3H), 1.62 - 1.54 (m, 2H). |
| (1S,2S)-N-(6-chloro-2-methylpyrimidin-4-yl)-2-(4- | |
| methylpyrimidin-2-yl)cyclopropane-1-carboxamide | |
| 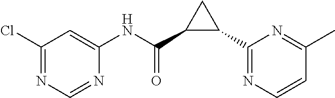 [SMILES: Cc1ccnc([C@H]2C[C@@H]2C(=O)Nc2cc(Cl)ncn2)n1] | ESI-MS [M + H]+: 290.2. ¹H NMR (400 MHz, DMSO) δ 11.60 (s, 1H), 8.75 (s, 1H), 8.53 (t, J = 6.9 Hz, 1H), 8.13 (s, 1H), 7.23 (d, J = 5.1 Hz, 1H), 2.72 - 2.68 (m, 1H), 2.63 - 2.57 (m, 1H), 2.42 (s, 3H), 1.63 - |
| (1S,2S)-N-(6-chloropyrimidin-4-yl)-2-(4- | 156 (m, 2H). |
| methylpyrimidin-2-yl)cyclopropane-1-carboxamide | |
| 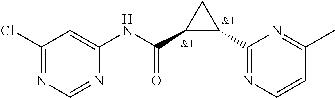 [SMILES: Cc1ccnc([C@H]2C[C@@H]2C(=O)Nc2cc(Cl)ncn2)n1] | ESI-MS [M + H]⁺: 290.2. |
| rac-(1S*,2S*)-N-(6-chloropyrimidin-4-yl)-2-(4- | |
| methylpyrimidin-2-yl)cyclopropane-1-carboxamide | |
| 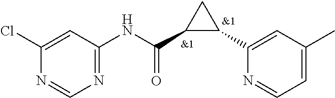 [SMILES: Cc1ccnc([C@H]2C[C@@H]2C(=O)Nc2cc(Cl)ncn2)c1] | ESI-MS [M + H]⁺: 289.2. |
| rac-(1S*,2S*)-N-(6-chloropyrimidin-4-yl)-2-(4- | |
| methylpyridin-2-yl)cyclopropane-1-carboxamide | |
| 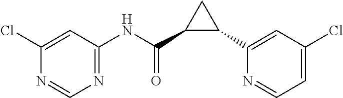 [SMILES: O=C(Nc1cc(Cl)ncn1)[C@H]1C[C@@H]1c1cc(Cl)ccn1] | ESI-MS [M + H]+: 309.2. ¹H NMR (400 MHz, DMSO) δ 11.58 (s, 1H), 8.75 (d, J = 0.9 Hz, 1H), 8.44 (d, J = 5.4 Hz, 1H), 8.13 (d, J = 0.9 Hz, 1H), 7.68 (d, J = 1.8 Hz, 1H), 7.37 |
| (1S,2S)-2-(4-chloropyridin-2-yl)-N-(6- | (dd, J = 5.4, 2.0 Hz, 1H), 2.72 - |
| chloropyrimidin-4-yl)cyclopropane-1-carboxamide | 2.63 (m, 2H), 1.63 - 1.53 (m, |
| | 2H). |
| 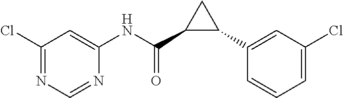 [SMILES: O=C(Nc1cc(Cl)ncn1)[C@H]1C[C@@H]1c1cccc(Cl)c1] | ESI-MS [M + H]+: 308.2. ¹H NMR (400 MHz, DMSO) δ 11.44 (s, 1H), 8.75 (d, J = 0.9 Hz, 1H), 8.14 (d, J = 0.9 Hz, 1H), 7.35- 7.30 (m, 3H), 7.18 (d, J = 7.5 Hz, 1H), 2.50 - 2.47 |
| (1S,2S)-2-(3-chlorophenyl)-N-(6-chloropyrimidin-4- | (m, 1H), 2.43 - 2.38 (m, 1H), |
| yl)cyclopropane-1-carboxamide | 1.57 - 1.51 (m, 2H). |
| 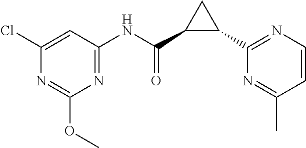 [SMILES: COc1nc(Cl)cc(NC(=O)[C@H]2C[C@@H]2c2nccc(C)n2)n1] | ESI-MS [M + H]+: 320.2. ¹H NMR (400 MHz, DMSO) δ 11.52 (s, 1H), 8.54 (d, J = 5.1 Hz, 1H), 7.77 (s, 1H), 7.23 (d, J = 5.1 Hz, 1H), 3.89 (s, 3H), 2.71 - 2.67 (m, 1H), 2.60 - 2.56 ( m, 1H), 2.42 (s, 3H), 1.62 - 1.55 (m, 2H). |
| (1S,2S)-N-(6-chloro-2-methoxypyrimidin-4-yl)-2-(4- | |
| methylpyrimidin-2-yl)cyclopropane-1-carboxamide | |
| 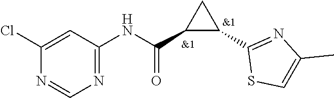 [SMILES: Cc1csc([C@H]2C[C@@H]2C(=O)Nc2cc(Cl)ncn2)n1] | ESI-MS [M + H]+: 295.1. |
| rac-(1S*,2S*)-N-(6-chloropyrimidin-4-yl)-2-(4- | |
| methylthiazol-2-yl)cyclopropane-1-carboxamide | |
| 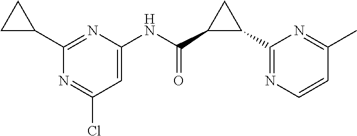 [SMILES: Cc1ccnc([C@H]2C[C@@H]2C(=O)Nc2cc(Cl)nc(C3CC3)n2)n1] | ESI-MS [M + H]+: 330.3. |
| (1S,2S)-N-(6-chloro-2-cyclopropylpyrimidin-4-yl)-2- | |
| (4-methylpyrimidin-2-yl)cyclopropane-1- | |
| carboxamide | |
| 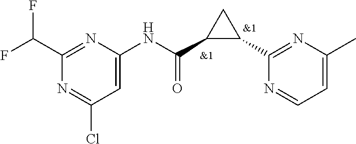 [SMILES: Cc1ccnc([C@H]2C[C@@H]2C(=O)Nc2cc(Cl)nc(C(F)F)n2)n1] | ESI-MS [M + H]+: 340.2. |
| rac-(1S*,2S*)-N-(6-chloro-2- | |
| (difluoromethyl)pyrimidin-4-yl)-2-(4- | |
| methylpyrimidin-2-yl)cyclopropane-1-carboxamide | |
| 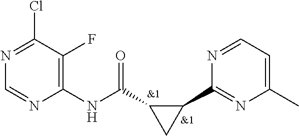 [SMILES: Cc1ccnc([C@H]2C[C@@H]2C(=O)Nc2ncnc(Cl)c2F)n1] | ESI-MS [M + H]+: 308.2 |
| rac-(1S*,2S*)-N-(6-chloro-5-fluoropyrimidin-4-yl)- | |
| 2-(4-methylpyrimidin-2-yl)cyclopropane-1- | |
| carboxamide | |
| 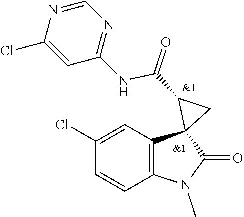 [SMILES: CN1C(=O)[C@]2(C[C@H]2C(=O)Nc2cc(Cl)ncn2)c2cc(Cl)ccc21] | ESI-MS [M + H]⁺: 363.2 |
| rac-(1R*,2S*)-5′-chloro-N-(6-chloropyrimidin-4-yl)- | |
| 1′-methyl-2′-oxospiro[cyclopropane-1,3′-indoline]-2- | |
| carboxamide | |

### Example 353

### Synthesis of rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide (I-353)

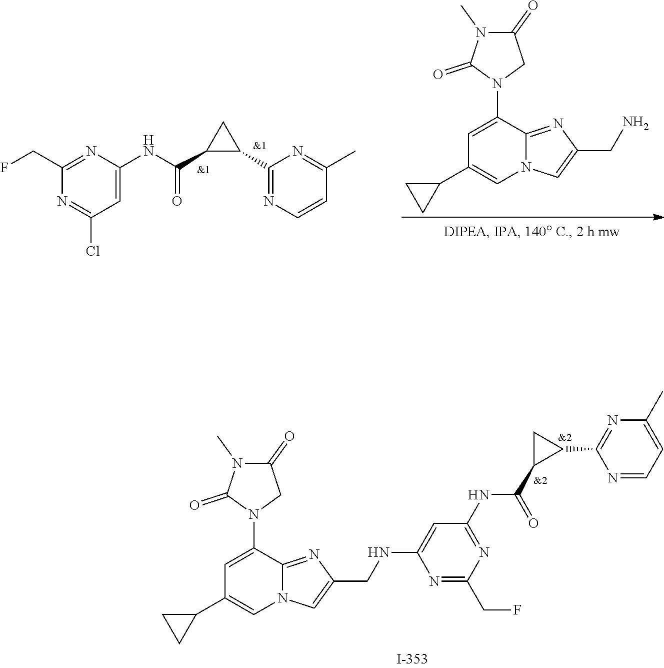

To a solution of 1-(2-(aminomethyl)-6-cyclopropylimidazo[1,2-a]pyridin-8-yl)-3-methylimidazolidine-2,4-dione (19 mg, 0.062 mmol) in IPA (3 mL) was added rac-(1S*,2S*)—N-(6-chloro-2-(fluoromethyl)pyrimidin-4-yl)-2-(4-methylpyrimidin-2-yl)cyclopropane-1-carboxamide (20 mg, 0.062 mmol) and DIPEA (24 mg, 0.19 mmol). Then the mixture was stirred in a sealed tube, then after degassing with N₂ for 1 min, the reaction was irradiated in a microwave reactor at 140° C. for 2 h. The mixture was cooled to room temperature, concentrated in vacuo and purified by preparative TLC (eluent: DCM/MeOH=20/1) to give the title compound as a white solid (13 mg, 36%). ESE-MS [M+H]+: 585.2, ¹H NMR (400 MHz, DMSO) δ 10.89 (s, 1H), 8.53 (d, J=5.1 Hz, 1H), 8.26 (s, 1H), 8.03 (s, 1H), 7.72 (s, 1H), 7.35 (s, 1H), 7.28 (s, 1H), 7.22 (d, J=5.1 Hz, 1H), 5.18 (d, J=46.9 Hz, 2H), 4.93 (s, 2H), 4.62 (s, 2H), 2.99 (s, 3H), 2.68-2.64 (m, 2H), 2.42 (s, 3H), 1.96-1.92 (m, 1H), 1.55-1.49 (m, 2H), 1.01-0.93 (m, 2H), 0.68-0.64 (m, 2H).

The compounds in Table 33 were prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide from a suitable amine and the indicated coupling partner.

TABLE 33

| | | Coupling Partner / |
|---|---|---|
| Example | Structure | Analytical Data |
| Example 354 | 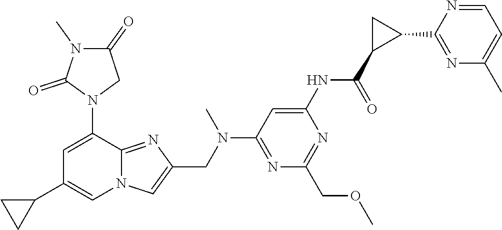 [SMILES: COCc1nc(NC(=O)[C@H]2C[C@@H]2c2nccc(C)n2)cc(N(C)Cc2cn3cc(C4CC4)cc(N4CC(=O)N(C)C4=O)c3n2)n1] | (1S,2S)-N-(6-chloro-2- (methoxymethyl)pyrimidin-4-yl)-2-(4- methylpyrimidin-2-yl)cyclopropane-1- carboxamide ESI-MS (M + H)+: 611.2, ¹H NMR (400 MHz, DMSO) δ 10.91 (s, 1H), 8.52 (d, J = 5.1 Hz, 1H), 8.21 (s, 1H), 7.68 (s, 1H), 7.36 (s, 2H), 7.20 (d, J = 5.1 Hz, 1H), 4.92-4.80 (m, 4H), 4.26 (s, 2H), 3.33 (s, 3H), 3.13 (s, 3H), 2.97 (s, 3H), 2.67-2.61 (m, 1H), 2.56-2.50 (m, 1H), 2.41 (s, 3H), 1.98-1.89 (m, 1H), 1.56-1.48 (m, 2H), 0.97-0.90 (m, 2H), 0.67-0.60 (m, 2H). |
| | (1S,2S)-N-(6-(((6-cyclopropyl-8- | |
| | (3-methyl-2,4-dioxoimidazolidin- | |
| | 1-yl)imidazo[1,2-a]pyridin-2- | |
| | yl)methyl)(methyl)amino)-2- | |
| | (methoxymethyl)pyrimidin-4-yl)- | |
| | 2-(4-methylpyrimidin-2- | |
| | yl)cyclopropane-1-carboxamide | |
| | (I-354) | |
| Example 355 | 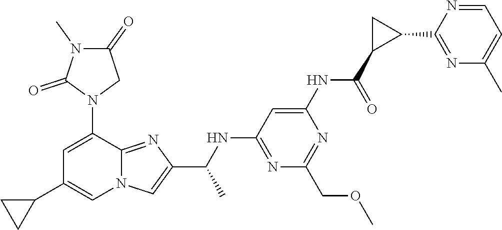 [SMILES: COCc1nc(NC(=O)[C@H]2C[C@@H]2c2nccc(C)n2)cc(N[C@H](C)c2cn3cc(C4CC4)cc(N4CC(=O)N(C)C4=O)c3n2)n1] | (1S,2S)-N-(6-chloro-2- (methoxymethyl)pyrimidin-4-yl)-2-(4- methylpyrimidin-2-yl)cyclopropane-1- carboxamide ESI-MS (M + H)+: 611.2, ¹H NMR (400 MHz, DMSO) δ 10.78 (s, 1H), 8.52 (d, J = 4.9 Hz, 1H), 8.23 (s, 1H), 7.78 (d, J = 7.8 Hz, 1H), 7.67 (s, 1H), 7.34 (s, 1H), 7.24-7.16 (m, 2H), 5.42 (s, 1H), 4.94 (s, 2H), 4.20 (s, 2H), 3.30 (s, 3H), 2.97 (s, 3H), 2.68-2.60 (m, 2H), 2.41 (s, 3H), 1.93 (s, 1H), 1.55-1.40 (m, 5H), 0.97-0.90 (m, 2H), 0.67-0.60 (m, 2H). |
| | (1S,2S)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-2- | |
| | (methoxymethyl)pyrimidin-4-yl)- | |
| | 2-(4-methylpyrimidin-2- | |
| | yl)cyclopropane-1-carboxamide | |
| | (I-355) | |
| Example 356 | 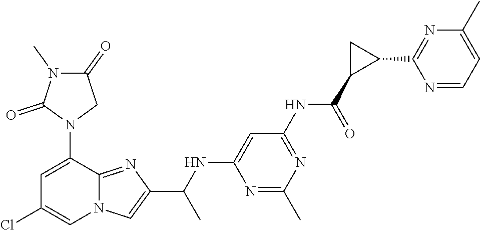 [SMILES: Cc1ccnc([C@H]2C[C@@H]2C(=O)Nc2cc(NC(C)c3cn4cc(Cl)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 575.2, ¹H NMR (400 MHz, DMSO) δ 10.66 (s, 1H), 8.67 (s, 1H), 8.52 (d, J = 5.1 Hz, 1H), 7.79 (s, 1H), 7.71 - 7.67 (m, 2H), 7.20 (d, J = 5.1 Hz, 1H), 7.14 (s, 1H), 5.65 - 5.09 (m, 1H), 5.03 (s, 2H), 2.98 (s, 3H), 2.61 - 2.56 (m, 2H), 2.41 (s, 3H), 2.25 (s, 3H), 1.53 - 1.46 (m, 5H). |
| | (1S,2S)-N-(6-((1-(6-chloro-8-(3- | |
| | methyl-2,4-dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-356) | |
| Example 357 | 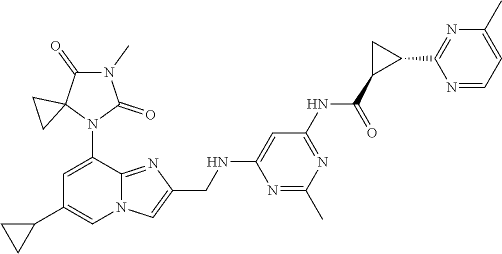 [SMILES: Cc1ccnc([C@H]2C[C@@H]2C(=O)Nc2cc(NCc3cn4cc(C5CC5)cc(N5C(=O)N(C)C(=O)C56CC6)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 593.3, ¹H NMR (400 MHz, DMSO) δ 10.67 (s, 1H), 8.52 (d, J = 5.1 Hz, 1H), 8.41 (d, J = 1.3 Hz, 1H), 7.76 (s, 1H), 7.68 (s, 1H), 7.20 (d, J = 5.1 Hz, 1H), 7.15 - 7.02 (m, 2H), 4.63 - 4.44 (m, 2H), 3.06 (s, 3H), 2.63 - 2.52 (m, 2H), 2.41 (s, 3H), 2.26 (s, 3H), 1.96 - 1.89 (m, 1H), 1.54 - 1.44 (m, 2H), 1.32 1.24 (m, 2H), 1.19 - 1.10 (m, 2H), 0.97 - 0.89 (m, 2H), 0.73 - 0.65 (m, 2H). |
| | (1S,2S)-N-(6-(((6-cyclopropyl-8- | |
| | (6-methyl-5,7-dioxo-4,6- | |
| | diazaspiro[2.4]heptan-4- | |
| | yl)imidazo[1,2-a]pyridin-2- | |
| | yl)methyl)amino)-2- | |
| | methylpyrimidin-4-yl)-2-(4- | |
| | methylpyrimidin-2- | |
| | yl)cyclopropane-1-carboxamide | |
| | (I-357) | |
| Example 358 | 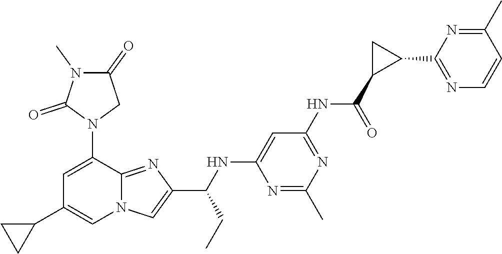 [SMILES: CC[C@@H](Nc1cc(NC(=O)[C@H]2C[C@@H]2c2nccc(C)n2)nc(C)n1)c1cn2cc(C3CC3)cc(N3CC(=O)N(C)C3=O)c2n1] | (1S,2S)-N-(6-chloro-2-methylpyrimidin-4- yl)-2-(4-methylpyrimidin-2-yl)cyclopropane- 1-carboxamide ESI-MS (M + H)+: 595.2, ¹H NMR (400 MHz, DMSO) δ 10.64 (s, 1H), 8.52 (d, J = 5.1 Hz, 1H), 8.23 (d, J = 1.1 Hz, 1H), 7.65 - 7.60 (m, 2H), 7.34 (d, J = 1.3 Hz, 1H), 7.20 - 7.17 (m, 2H), 5.30 - 5.24 (m, 1H), 4.96 - 4.95 (m, 2H), 2.98 (s, 3H), 2.59 - 2.57 (m, 1H), 2.54 - 2.52 (m, 1H), 2.41 (s, 3H), 2.24 (s, 3H), 2.02 - 1.91 (m, 2H), 1.83 - 1.77 (m, 1H), 1.52 - 1.47 (m, 2H), 0.95 - 0.89 (m, 5H), 0.65 - 0.62 (m, 2H). |
| | (1S,2S)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)propyl)amino)-2- | |
| | methylpyrimidin-4-yl)-2-(4- | |
| | methylpyrimidin-2- | |
| | yl)cyclopropane-1-carboxamide | |
| | (I-358) | |
| Example 359 | 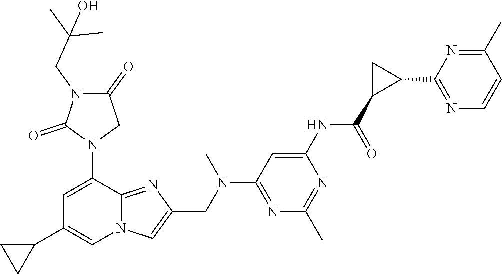 [SMILES: Cc1ccnc([C@H]2C[C@@H]2C(=O)Nc2cc(N(C)Cc3cn4cc(C5CC5)cc(N5CC(=O)N(CC(C)(C)O)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 639.3, ¹H NMR (400 MHz, DMSO) δ 10.75 (s, 1H), 8.49 (d, J = 5.1 Hz, 1H), 8.17 (s, 1H), 7.63 (s, 1H), 7.35 (d, J = 1.4 Hz, 1H), 7.26 (s, 1H), 7.17 (d, J = 5.1 Hz, 1H), 4.90 (s, 2H), 4.78 (s, 2H), 4.56 (s, 1H), 3.41 (s, 2H), 3.08 (s, 3H), 2.60 - 2.55 (m, 1H), 2.52 - 2.49 (m, 1H), 2.38 (s, 3H), 2.27 (s, 3H), 1.94 - 1.88 (m, 1H), 1.51 - 1.44 (m, 2H), 1.10 (s, 6H), 0.92 - 0.88 (m, 2H), 0.62 - 0.58 (m, 2H) |
| | (1S,2S)-N-(6-(((6-cyclopropyl-8- | |
| | (3-(2-hydroxy-2-methylpropyl)- | |
| | 2,4-dioxoimidazolidin-1- | |
| | yl)imidazo[1,2-a]pyridin-2- | |
| | yl)methyl)(methyl)amino)-2- | |
| | methylpyrimidin-4-yl)-2-(4- | |
| | methylpyrimidin-2- | |
| | yl)cyclopropane-1-carboxamide | |
| | (I-359) | |
| Example 360 | 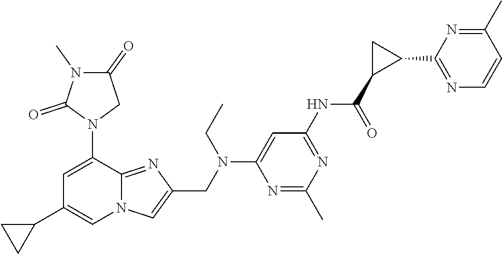 [SMILES: CCN(Cc1cn2cc(C3CC3)cc(N3CC(=O)N(C)C3=O)c2n1)c1cc(NC(=O)[C@H]2C[C@@H]2c2nccc(C)n2)nc(C)n1] | (1S,2S)-N-(6-chloro-2-methylpyrimidin-4- yl)-2-(4-methylpyrimidin-2-yl)cyclopropane- 1-carboxamide ESI-MS (M + H)+: 595.2, ¹H NMR (400 MHz, DMSO) δ = 10.74 (s, 1H), 8.51 (d, J = 5.1, 1H), 8.22 (d, J = 1.1, 1H), 7.68 (s, 1H), 7.36 (d, J = 1.4, 1H), 7.29 (s, 1H), 7.20 (d, J = 5.1, 1H), 4.91 (s, 2H), 4.73 (s, 2H), 3.59 (s, 2H), 2.97 (s, 3H), 2.61-2.56 (m, 1H), 2.54-2.51 (m, 1H), 2.41 (s, 3H), 2.30 (s, 3H), 1.97-1.90 (m, 1H), 1.55-1.45 (m, 2H), 1.16-1.12 (m, 3H), 0.98- 0.89 (m, 2H), 0.68 - 0.59 (m, 2H). |
| | (1S,2S)-N-(6-(((6-cyclopropyl-8- | |
| | (3-methyl-2,4-dioxoimidazolidin- | |
| | 1-yl)imidazo[1,2-a]pyridin-2- | |
| | yl)methyl)(ethyl)amino)-2- | |
| | methylpyrimidin-4-yl)-2-(4- | |
| | methylpyrimidin-2- | |
| | yl)cyclopropane-1-carboxamide | |
| | (I-360) | |
| Example 361 | 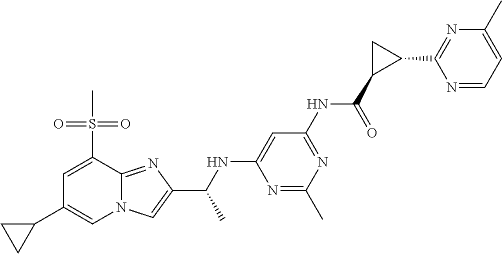 [SMILES: Cc1ccnc([C@H]2C[C@@H]2C(=O)Nc2cc(N[C@H](C)c3cn4cc(C5CC5)cc(S(C)(=O)=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 547.2, ¹H NMR (400 MHz, DMSO) δ 10.67 (s, 1H), 8.64 (s, 1H), 8.52 (d, J = 5.1 Hz, 1H), 7.84 - 7.67 (m, 2H), 7.56 (s, 1H), 7.20 (d, J = 5.1 Hz, 1H), 7.14 (s, 1H), 5.47 (s, 1H), 3.55 (s, 3H), 2.64 - 2.56 (m, 2H), 2.41 (s, 3H), 2.24 (s, 3H), 2.10 - 2.03 (m, 1H), 1.54 - 1.44 (m, 5H), 1.04 - 0.90 (m, 2H), 0.72 - 0.69 (m, 2H). |
| | (1S,2S)-N-(6-(((R)-1-(6- | |
| | cyclopropyl-8- | |
| | (methylsulfonyl)imidazo[1,2- | |
| | a]pyridin-2-yl)ethyl)amino)-2- | |
| | methylpyrimidin-4-yl)-2-(4- | |
| | methylpyrimidin-2- | |
| | yl)cyclopropane-1-carboxamide | |
| | (I-361) | |
| Example 362 | 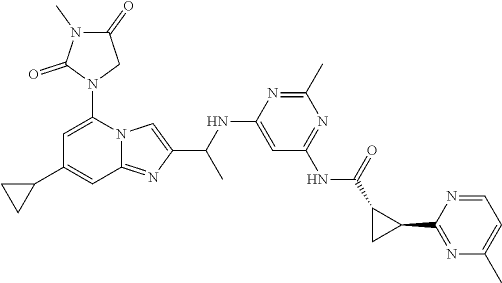 [SMILES: Cc1ccnc([C@H]2C[C@@H]2C(=O)Nc2cc(NC(C)c3cn4c(N5CC(=O)N(C)C5=O)cc(C5CC5)cc4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 581.2, ¹H NMR (400 MHz, cd3od) δ 8.47 - 8.45 (m, 1H), 7.68 (s, 1H), 7.25 (s, 1H), 7.16 - 7.15 (m, 1H), 7.10 (d, J = 4.9 Hz, 1H), 6.79 - 6.78 (m, 1H), 4.60 (s, 2H), 4.53 - 4.49 (m, 1H), 3.09 (s, 3H), 2.70 - 2.65 (m, 1H), 2.51 - 2.46 (m, 4H), 2.30 (s, 3H), 2.05 - 2.02 (m, 1H), 1.66 - 1.60 (m, 5H), 1.09 - 1.07 (m, 2H), 0.84 - 0.82 (m, 2H). |
| | (1S,2S)-N-(6-((1-(7-cyclopropyl- | |
| | 5-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-362) | |
| Example 363 | 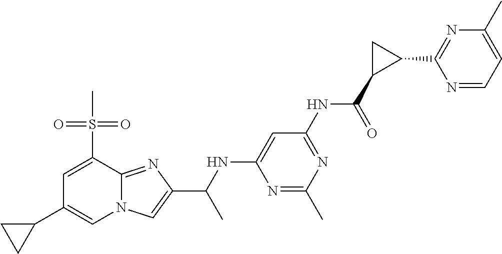 [SMILES: Cc1ccnc([C@H]2C[C@@H]2C(=O)Nc2cc(NC(C)c3cn4cc(C5CC5)cc(S(C)(=O)=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 547.2, ¹H NMR (400 MHz, DMSO) δ 10.67 (s, 1H), 8.64 (s, 1H), 8.52 (d, J = 5.1 Hz, 1H), 8.39 (s, 1H), 7.79 - 7.73 (m, 2H), 7.56 (s, 1H), 7.20 (d, J = 5.1 Hz, 1H), 7.14 (s, 1H), 5.54 - 5.46 (m, 1H), 3.55 (s, 3H), 2.58 - 2.56 (m, 2H), 2.41 (s, 3H), 2.24 (s, 3H), 2.13 - 2.01 (m, 1H), 1.51 (m, 5H), 0.98 - 0.96 (m, 2H), 0.71 - 0.70 (m, 2H). |
| | (1S,2S)-N-(6-((1-(6-cyclopropyl- | |
| | 8-(methylsulfonyl)imidazo[1,2- | |
| | a]pyridin-2-yl)ethyl)amino)-2- | |
| | methylpyrimidin-4-yl)-2-(4- | |
| | methylpyrimidin-2- | |
| | yl)cyclopropane-1-carboxamide | |
| | (I-363) | |
| Example 364 | 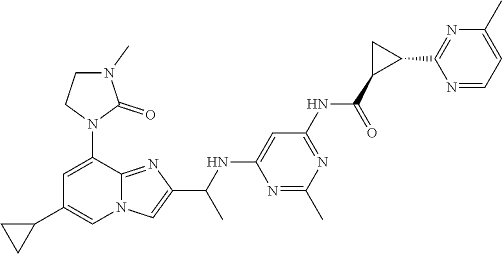 [SMILES: Cc1ccnc([C@H]2C[C@@H]2C(=O)Nc2cc(NC(C)c3cn4cc(C5CC5)cc(N5CCN(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 567.2, ¹H NMR (400 MHz, DMSO) δ 10.65 (s, 1H), 8.52 (d, J = 5.1 Hz, 1H), 8.11 - 8.07 (m, 1H), 7.65 - 7.57 (m, 2H), 7.24 - 7.18 (m, 2H), 7.13 (s, 1H), 5.55 - 5.30 (m, 1H), 4.34 - 4.28 (m, 2H), 3.50 - 3.44 (m, 2H), 2.79 (s, 3H), 2.63 - 2.53 (m, 2H), 2.41 (s, 3H), 2.24 (s, 3H), 1.91 - 1.86 (m, 1H), 1.57 - 1.42 (m, 5H), 0.93 - 0.87 (m, 2H), 0.64 - 0.57 (m, 2H). |
| | (1S,2S)-N-(6-((1-(6-cyclopropyl- | |
| | 8-(3-methyl-2-oxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-364) | |
| Example 365 | 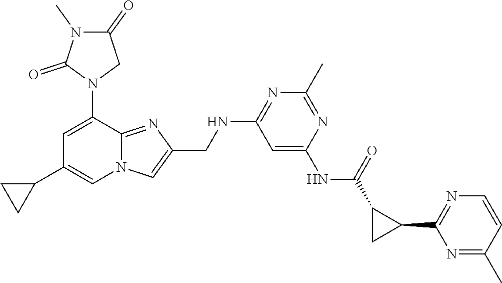 [SMILES: Cc1ccnc([C@H]2C[C@@H]2C(=O)Nc2cc(NCc3cn4cc(C5CC5)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 567.2, ¹H NMR (400 MHz, DMSO) δ 10.65 (s, 1H), 8.51 (d, J = 5.1 Hz, 1H), 8.25 (d, J = 0.7 Hz, 1H), 7.71 (s, 1H), 7.69 (s, 1H), 7.33 (d, J = 1.0 Hz, 1H), 7.20 (d, J = 5.1 Hz, 1H), 7.13 (s, 1H), 4.91 (s, 2H), 4.56 (s, 2H), 2.98 (s, 3H), 2.62-2.57 (m, 1H), 2.55-2.50 (m, 1H),2.41 (s, 3H), 2.27 (s, 3H), 1.97-1.90 (m, 1H), 1.54 - 1.46 (m, 2H), 0.96- 0.91 (m, 2H), 0.66- 0.62 (m, 2H). |
| | (1S,2S)-N-(6-(((6-cyclopropyl-8- | |
| | (3-methyl-2,4-dioxoimidazolidin- | |
| | 1-yl)imidazo[1,2-a]pyridin-2- | |
| | yl)methyl)amino)-2- | |
| | methylpyrimidin-4-yl)-2-(4- | |
| | methylpyrimidin-2- | |
| | yl)cyclopropane-1-carboxamide | |
| | (I-365) | |
| Example 366 | 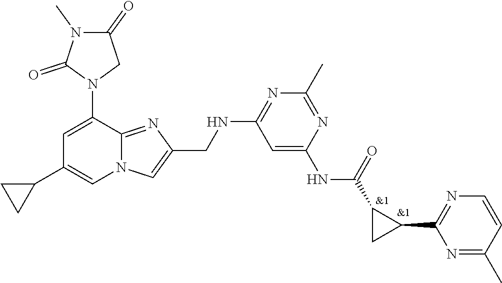 [SMILES: Cc1ccnc([C@H]2C[C@@H]2C(=O)Nc2cc(NCc3cn4cc(C5CC5)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS (M + H)+: 567.2, ¹H NMR (400 MHz, DMSO) δ 10.67 (s, 1H), 8.52 (d, J = 5.1 Hz, 1H), 8.25 (d, J = 1.0 Hz, 1H), 7.73 (s, 1H), 7.69 (s, 1H), 7.33 (d, J = 1.2 Hz, 1H), 7.20 (d, J = 5.1 Hz, 1H), 7.14 (s, 1H), 4.92 (s, 2H), 4.57 (s, 2H), 2.98 (s, 3H), 2.61 - 2.57 (m, 1H), 2.54 - 2.51 (m, 1H), 2.41 (s, 3H), 2.27 (s, 3H), 1.97 - 1.90 (m, 1H), 1.54 - 1.45 (m, 2H), 0.96 - 0.91 (m, 2H), 0.66 - 0.62 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-2- | |
| | methylpyrimidin-4-yl)-2-(4- | |
| | methylpyrimidin-2- | |
| | yl)cyclopropane-1-carboxamide | |
| | (I-366) | |
| Example 367 | 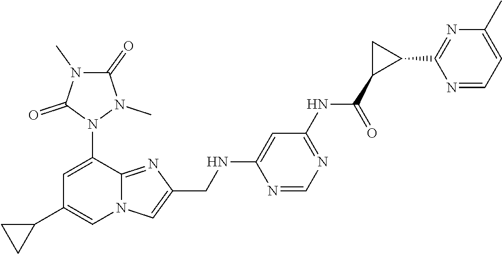 [SMILES: Cc1ccnc([C@H]2C[C@@H]2C(=O)Nc2cc(NCc3cn4cc(C5CC5)cc(-n5c(=O)n(C)c(=O)n5C)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 568.2, ¹H NMR (400 MHz, DMSO) δ 10.69 (s, 1H), 8.52 (d, J = 5.1 Hz, 1H), 8.45 (s, 1H), 8.18 (s, 1H), 7.94 (s, 1H), 7.70 (s, 1H), 7.28 (s, 1H), 7.27 - 7.16 (m, 2H), 4.55 (s, 2H), 3.04 (s, 3H), 2.95 (s, 3H), 2.66 - 2.56 (m, 2H), 2.41 (s, 3H), 2.02 - 1.92 (m, 1H), 1.56 - 1.49 (m, 2H), 0.99 - 0.89 (m, 2H), 0.75 - 0.67 (m, 2H). |
| | (1S,2S)-N-(6-(((6-cyclopropyl-8- | |
| | (2,4-dimethyl-3,5-dioxo-1,2,4- | |
| | triazolidin-1-yl)imidazo[1,2- | |
| | a]pyridin-2- | |
| | yl)methyl)amino)pyrimidin-4-yl)- | |
| | 2-(4-methylpyrimidin-2- | |
| | yl)cyclopropane-1-carboxamide | |
| | (I-367) | |
| Example 368 | 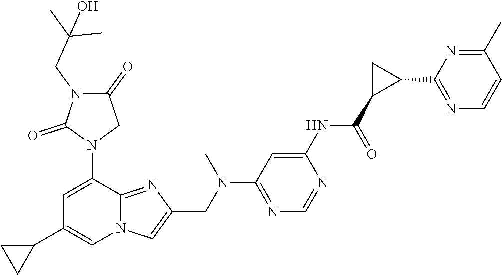 [SMILES: Cc1ccnc([C@H]2C[C@@H]2C(=O)Nc2cc(N(C)Cc3cn4cc(C5CC5)cc(N5CC(=O)N(CC(C)(C)O)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 625.3, ¹H NMR (400 MHz, DMSO) δ 10.75 (s, 1H), 8.50 (d, J = 5.1 Hz, 1H), 8.24 (d, J = 0.8 Hz, 1H), 8.18 (s, 1H), 7.65 (s, 1H), 7.43 (s, 1H), 7.36 (d, J = 1.4 Hz, 1H), 7.18 (d, J = 5.1 Hz, 1H), 4.91 (s, 2H), 4.80 (s, 2H), 4.55 (s, 1H), 3.42 (s, 2H), 3.11 (s, 3H), 2.64 - 2.59 (m, 1H), 2.53 - 2.52 (m, 1H), 2.39 (s, 3H), 1.94 - 1.89 (m, 1H), 1.53 - 1.48 (m, 2H), 1.11 (s, 6H), 0.93 - 0.89 (m, 2H), 0.63 - 0.59 (m, 2H). |
| | (1S,2S)-N-(6-(((6-cyclopropyl-8- | |
| | (3-(2-hydroxy-2-methylpropyl)- | |
| | 2,4-dioxoimidazolidin-1- | |
| | yl)imidazo[1,2-a]pyridin-2- | |
| | yl)methyl)(methyl)amino)pyrimidin- | |
| | 4-yl)-2-(4-methylpyrimidin-2- | |
| | yl)cyclopropane-1-carboxamide | |
| | (I-368) | |
| Example 369 | 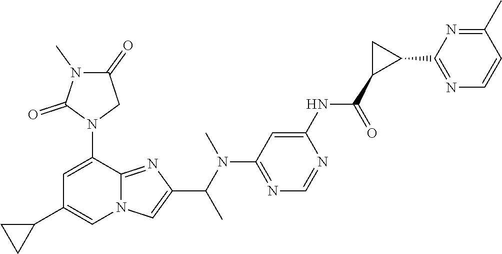 [SMILES: Cc1ccnc([C@H]2C[C@@H]2C(=O)Nc2cc(N(C)C(C)c3cn4cc(C5CC5)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 581.3, ¹H NMR (400 MHz, DMSO) δ 10.80 (s, 1H), 8.52 (d, J = 5.1 Hz, 1H), 8.28 (d, J = 0.7 Hz, 1H), 8.20 (s, 1H), 7.76 (s, 1H), 7.35 (s, 2H), 7.20 (d, J = 5.1 Hz, 1H), 4.87 (s, 2H), 3.88 (d, J = 0.9 Hz, 1H), 2.95 (d, J = 1.2 Hz, 3H), 2.80 (s, 3H), 2.67 - 2.62 (m, 1H), 2.56 - 2.53 (m, 1H), 2.41 (s, 3H), 1.98 - 1.91 (m, 1H), 1.55 - 1.49 (m, 5H), 0.96 - 0.91 (m, 2H), 0.65 - 0.61 (m, 2H). |
| | (1S,2S)-N-(6-((1-(6-cyclopropyl- | |
| | 8-(3-methyl-2,4- | |
| | dioxoimidazolidin-1- | |
| | yl)imidazo[1,2-a]pyridin-2- | |
| | yl)ethyl)(methyl)amino)pyrimidin- | |
| | 4-yl)-2-(4-methylpyrimidin-2- | |
| | yl)cyclopropane-1-carboxamide | |
| | (I-369) | |
| Example 370 | 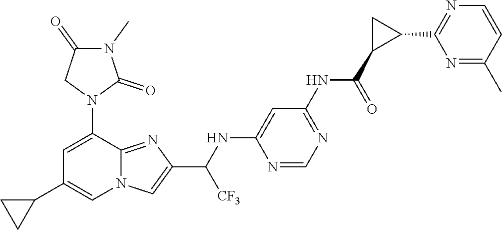 [SMILES: Cc1ccnc([C@H]2C[C@@H]2C(=O)Nc2cc(NC(c3cn4cc(C5CC5)cc(N5CC(=O)N(C)C5=O)c4n3)C(F)(F)F)ncn2)n1] | (1S,2S)-N-(6-chloropyrimidin-4-yl)-2-(4- methylpyrimidin-2-yl)cyclopropane-1- carboxamide ESI-MS (M + H)+: 621.2, ¹H NMR (400 MHz, DMSO) δ 10.82 (s, 1H), 8.54 - 8.46 (m, 2H), 8.33 - 8.30 (m, 2H), 8.06 (s, 1H), 7.54 (s, 1H), 7.44 (s, 1H), 7.22 - 7.20 (m, 1H), 6.38 - 6.34 (m, 1H), 4.98 (s, 2H), 2.98 (s, 3H), 2.65 - 2.63 (m, 1H), 2.59 - 2.55 (m, 1H), 2.42 - 2.41(m, 3H), 1.99 - 1.95 (m, 1H), 1.57 - 1.51 (m, 2H), 0.99 - 0.94 (m, 2H), 0.69 - 0.65 (m, 2H). |
| | (1S,2S)-N-(6-((1-(6-cyclopropyl- | |
| | 8-(3-methyl-2,4- | |
| | dioxoimidazolidin-1- | |
| | yl)imidazo[1,2-a]pyridin-2-yl)- | |
| | 2,2,2- | |
| | trifluoroethyl)amino)pyrimidin-4- | |
| | yl)-2-(4-methylpyrimidin-2- | |
| | yl)cyclopropane-1-carboxamide | |
| | (I-370) | |
| Example 371 | 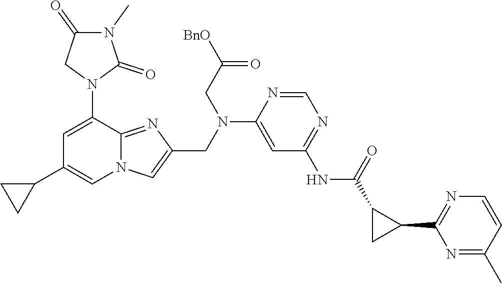 [SMILES: Cc1ccnc([C@H]2C[C@@H]2C(=O)Nc2cc(N(CC(=O)OCc3ccccc3)Cc3cn4cc(C5CC5)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 701.2, ¹H NMR (400 MHz, DMSO) δ 10.82 (s, 1H), 8.52 (d, J = 4.7 Hz, 1H), 8.31 - 8.17 (m, 2H), 7.92 - 7.66 (m, 2H), 7.43 - 7.28 (m, 6H), 7.20 (d, J = 4.7 Hz, 1H), 5.14 (s, 2H), 4.93 (s, 2H), 4.78 - 4.45 (m, 4H), 2.97 (s, 3H), 2.66 - 2.58 (m, 1H), 2.57 - 2.50 (s, 1H), 2.41 (s, 3H), 2.00 - 1.85 (m, 1H), 1.60 - 1.44 (m, 2H), 1.02 - 0.87 (m, 2H), 0.73 - 0.59 (m, 2H). |
| | benzyl N-((6-cyclopropyl-8-(3- | |
| | methyl-2,4-dioxoimidazolidin-1- | |
| | yl)imidazo[1,2-a]pyridin-2- | |
| | yl)methyl)-N-(6-((1S,2S)-2-(4- | |
| | methylpyrimidin-2- | |
| | yl)cyclopropane-1- | |
| | carboxamido)pyrimidin-4- | |
| | yl)glycinate (I-371) | |
| Example 372 | 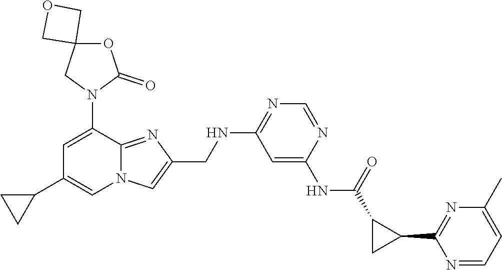 [SMILES: Cc1ccnc([C@H]2C[C@@H]2C(=O)Nc2cc(NCc3cn4cc(C5CC5)cc(N5CC6(COC6)OC5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 568.2, ¹H NMR (400 MHz, DMSO) δ 10.69 (s, 1H), 8.52 (d, J = 5.1 Hz, 1H), 8.25 (s, 1H), 8.19 (s, 1H), 7.90 (s, 1H), 7.69 (s, 1H), 7.31 (s, 1H), 7.26 - 7.18 (m, 2H), 4.87-4.78 (m, 4H), 4.72 (s, 2H), 4.59 (s, 2H), 2.65 - 2.59 (m, 1H), 2.56-2.51 (m, 1H), 2.41 (s, 3H), 1.94 - 1.90 (m, 1H), 1.57 - 1.47 (m, 2H), 0.96 - 0.89 (m, 2H), 0.67 - 0.59 (m, 2H). ESI-MS [M + H] +: 568.2 |
| | (1S,2S)-N-(6-(((6-cyclopropyl-8- | |
| | (6-oxo-2,5-dioxa-7- | |
| | azaspiro[3.4]octan-7- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-372) | |
| Example 373 | 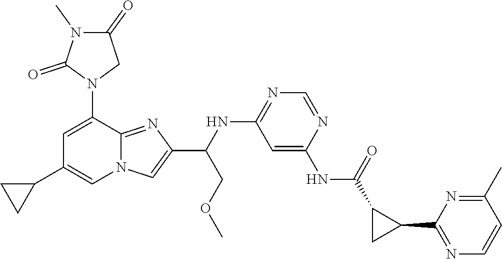 [SMILES: COCC(Nc1cc(NC(=O)[C@H]2C[C@@H]2c2nccc(C)n2)ncn1)c1cn2cc(C3CC3)cc(N3CC(=O)N(C)C3=O)c2n1] | (1S,2S)-N-(6-chloropyrimidin-4-yl)-2-(4- methylpyrimidin-2-yl)cyclopropane-1- carboxamide ESI-MS (M + H)+: 597.2, ¹H NMR (400 MHz, DMSO) δ 10.66 (s, 1H), 8.52 (d, J = 5.1 Hz, 1H), 8.24 (s, 1H), 8.16 (s, 1H), 7.86 (s, 1H), 7.71 (d, J = 2.0 Hz, 1H), 7.35 (s, 2H), 7.21 (d, J = 5.1 Hz, 1H), 5.51 (s, 1H), 4.95 (s, 2H), 3.79 - 3.73 (m, 1H), 3.71 - 3.65 (m, 1H), 3.28 (s, 3H), 2.98 (s, 3H), 2.64 - 2.60 (m, 1H), 2.57 - 2.53 (m, 1H), 2.41 (s, 3H), 1.97 - 1.90 (m, 1H), 1.57 - 1.48 (m, 2H), 0.96 - 0.90 (m, 2H), 0.66 - 0.61 (m, 2H). |
| | (1S,2S)-N-(6-((1-(6-cyclopropyl- | |
| | 8-(3-methyl-2,4- | |
| | dioxoimidazolidin-1- | |
| | yl)imidazo[1,2-a]pyridin-2-yl)-2- | |
| | methoxyethyl)amino)pyrimidin-4- | |
| | yl)-2-(4-methylpyrimidin-2- | |
| | yl)cyclopropane-1-carboxamide | |
| | (I-373) | |
| Example 374 | 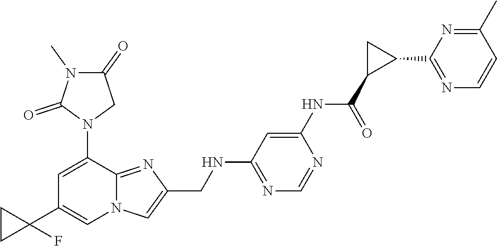 [SMILES: Cc1ccnc([C@H]2C[C@@H]2C(=O)Nc2cc(NCc3cn4cc(C5(F)CC5)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 571.2, ¹H NMR (400 MHz, DMSO) δ 10.69 (s, 1H), 8.60 - 8.46 (m, 2H), 8.19 (s, 1H), 7.94 (s, 1H), 7.79 (s, 1H), 7.59 (s, 1H), 7.32 (s, 1H), 7.21 (d, J = 5.1 Hz, 1H), 4.95 (s, 2H), 4.61 (s, 2H), 2.98 (s, 3H), 2.69 - 2.58 (m, 2H), 2.41 (s, 3H), 1.61 - 1.39 (m, 4H), 1.16-1.08 (m, 2H). |
| | (1S,2S)-N-(6-(((6-(1- | |
| | fluorocyclopropyl)-8-(3-methyl- | |
| | 2,4-dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-374) | |
| Example 375 | 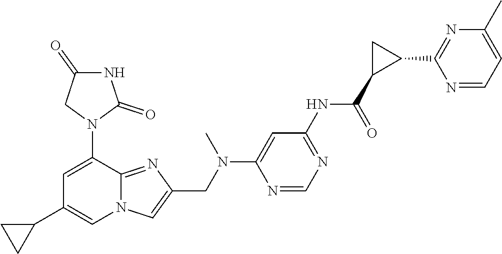 [SMILES: Cc1ccnc([C@H]2C[C@@H]2C(=O)Nc2cc(N(C)Cc3cn4cc(C5CC5)cc(N5CC(=O)NC5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 553.3, ¹H NMR (400 MHz, DMSO) δ 11.32 (s, 1H), 10.77 (s, 1H), 8.52 (d, J = 5.1 Hz, 1H), 8.26 (s, 1H), 8.18 (s, 1H), 7.65 (s, 1H), 7.45 (s, 1H), 7.35 (d, J = 1.1 Hz, 1H), 7.20 (d, J = 5.1 Hz, 1H), 4.84 (d, J = 19.7 Hz, 4H), 3.13 (s, 3H), 2.64 (s, 1H), 2.57 - 2.53 (m, 1H), 2.41 (s, 3H), 1.95-1.89 (m, 1H), 1.54-1.51 (m, 2H), 0.95-0.90 (m, 2H), 0.67 - 0.58 (m, 2H). |
| | (1S,2S)-N-(6-(((6-cyclopropyl-8- | |
| | (2,4-dioxoimidazolidin-1- | |
| | yl)imidazo[1,2-a]pyridin-2- | |
| | yl)methyl)(methyl)amino)pyrimi- | |
| | din-4-yl)-2-(4-methylpyrimidin-2- | |
| | yl)cyclopropane-1-carboxamide | |
| | (I-375) | |
| Example 376 | 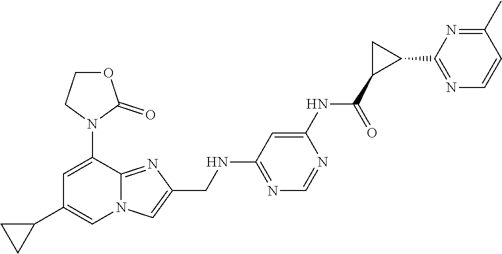 [SMILES: Cc1ccnc([C@H]2C[C@@H]2C(=O)Nc2cc(NCc3cn4cc(C5CC5)cc(N5CCOC5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 526.2, ¹H NMR (400 MHz, DMSO) δ 10.65 (s, 1H), 8.50 (d, J = 5.1 Hz, 1H), 8.22 (s, 1H), 8.16 (s, 1H), 7.86 (s, 1H), 7.65 (s, 1H), 7.27 (s, 1H), 7.18 (d, J = 5.4 Hz, 2H), 4.55 - 4.42 (m, 6H), 2.62 - 2.57 (m, 1H), 2.54 - 2.52 (m, 1H), 2.39 (s, 3H), 1.93 - 1.87 (m, 1H), 1.53 - 1.45 (m, 2H), 0.92 - 0.87 (m, 2H), 0.64 - 0.60 (m, 2H). |
| | (1S,2S)-N-(6-(((6-cyclopropyl-8- | |
| | (2-oxooxazolidin-3- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-376) | |
| Example 377 | 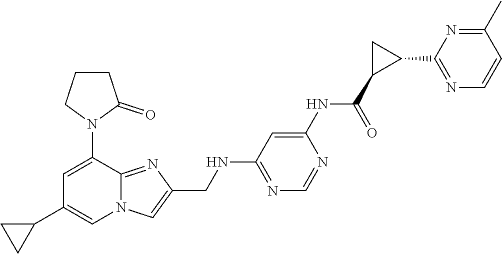 [SMILES: Cc1ccnc([C@H]2C[C@@H]2C(=O)Nc2cc(NCc3cn4cc(C5CC5)cc(N5CCCC5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 524.1, ¹H NMR (400 MHz, DMSO) δ 10.64 (s, 1H), 8.50 (d, J = 5.1 Hz, 1H), 8.21 (d, J = 1.2 Hz, 1H), 8.16 (s, 1H), 7.85 (s, 1H), 7.63 (s, 1H), 7.27 (s, 1H), 7.18 (d, J = 5.1 Hz, 1H), 7.09 (d, J = 1.2 Hz, 1H), 4.54 (s, 1H), 4.12 (t, J = 7.1 Hz, 2H), 2.62 - 2.58 (m, 1H), 2.53 - 2.51 (m, 1H), 2.45 (s, 1H), 2.43 (s, 1H), 2.39 (s, 3H), 2.13 - 2.06 (m, 2H), 1.92 - 1.86 (m, 1H), 1.54 - 1.45 (m, 2H), 0.92 - 0.87 (m, 2H), 0.63 - 0.59 (m, 2H). |
| | (1S,2S)-N-(6-(((6-cyclopropyl-8- | |
| | (2-oxopyrrolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-377) | |
| Example 378 | 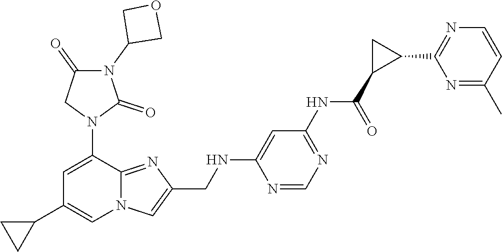 [SMILES: Cc1ccnc([C@H]2C[C@@H]2C(=O)Nc2cc(NCc3cn4cc(C5CC5)cc(N5CC(=O)N(C6COC6)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 595.3, ¹H NMR (400 MHz, DMSO) δ 10.65 (s, 1H), 8.50 (d, J = 5.1 Hz, 1H), 8.23 (d, J = 1.1 Hz, 1H), 8.16 (s, 1H), 7.86 (s, 1H), 7.67 (s, 1H), 7.30 (d, J = 9.0 Hz, 2H), 7.19 (d, J = 5.1 Hz, 1H), 5.19 - 5.13 (m, 1H), 5.11 - 5.08 (m, 2H), 4.89 (s, 2H), 4.71 - 4.67 (m, 2H), 4.55 (s, 2H), 2.61 - 2.57 (m, 1H), 2.54 - 2.53 (m, 1H), 2.39 (s, 3H), 1.95 - 1.88 (m, 1H), 1.53 - 1.46 (m, 2H), 0.94 - 0.89 (m, 2H), 0.64 - 0.60 (m, 2H). |
| | (1S,2S)-N-(6-(((6-cyclopropyl-8- | |
| | (3-(oxetan-3-yl)-2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-378) | |
| Example 379 | 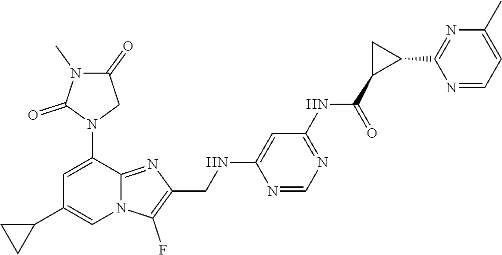 [SMILES: Cc1ccnc([C@H]2C[C@@H]2C(=O)Nc2cc(NCc3nc4c(N5CC(=O)N(C)C5=O)cc(C5CC5)cn4c3F)ncn2)n1] | (1S,2S)-N-(6-chloropyrimidin-4-yl)-2-(4- methylpyrimidin-2-yl)cyclopropane-1- carboxamide ESI-MS (M + H)+: 571.2, ¹H NMR (400 MHz, DMSO) δ 10.66 (s, 1H), 8.52 (d, J = 5.1 Hz, 1H), 8.20 - 8.19 (m, 1H), 7.96 (d, J = 1.2 Hz, 1H), 7.89 (s, 1H), 7.34 (d, J = 1.3 Hz, 1H), 7.30 (s, 1H), 7.20 (d, J = 5.1 Hz, 1H), 4.90 (s, J = 8.7 Hz, 2H), 4.58 (s, 2H), 2.97 (s, 3H), 2.68 - 2.54 (m, 2H), 2.41 (s, 3H), 2.04 - 1.98 (m, 1H), 1.55 - 1.47 (m, 2H), 0.98 - 0.93 (m, 2H), 0.73 - 0.69 (m, 2H). |
| | (1S,2S)-N-(6-(((6-cyclopropyl-3- | |
| | fluoro-8-(3-methyl-2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-379) | |
| Example 380 | 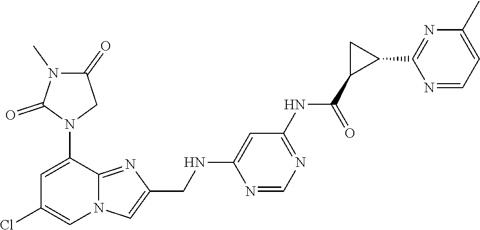 [SMILES: Cc1ccnc([C@H]2C[C@@H]2C(=O)Nc2cc(NCc3cn4cc(Cl)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 547.2, ¹H NMR (400 MHz, cd3od) δ 8.49 - 8.44 (m, 2H), 8.17 (d, J = 4.8 Hz, 1H), 7.78 (s, 1H), 7.68 (d, J = 1.6 Hz, 1H), 7.39 (s, 1H), 7.17 (d, J = 5.2 Hz, 1H), 4.87 (s, 2H), 4.67 (s, 2H), 3.10 (s, 3H), 2.72 - 2.65 (m, 1H), 2.64 - 2.56 (m, 1H), 2.47 (s, 3H), 1.70 - 1.62 (m, 2H). |
| | (1S,2S)-N-(6-(((6-chloro-8-(3- | |
| | methyl-2,4-dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-380) | |
| Example 381 | 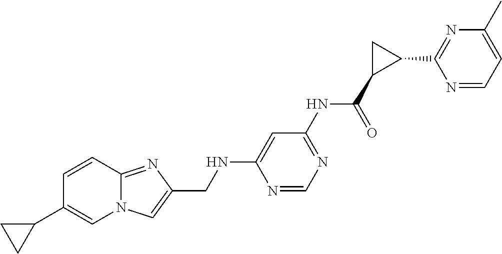 [SMILES: Cc1ccnc([C@H]2C[C@@H]2C(=O)Nc2cc(NCc3cn4cc(C5CC5)ccc4n3)ncn2)n1] | (1S,2S)-N-(6-chloropyrimidin-4-yl)-2-(4- methylpyrimidin-2-yl)cyclopropane-1- carboxamide ESI-MS (M + H)+: 441.1, ¹H NMR (400 MHz, MeOD) δ 8.47 (d, J = 5.2 Hz, 2H), 8.31 (s, 1H), 7.90 (s, 1H), 7.82 (s, 1H), 7.42 (s, 1H), 7.29 (d, J = 10.0 Hz, 1H), 7.17 (d, J = 5.2 Hz, 1H), 4.75 (s, 2H), 2.72 - 2.67 (m, 1H), 2.57 - 2.52 (m, 1H), 2.47 (s, 3H), 2.03 - 1.95 (m, 1H), 1.71 - 1.62 (m, 2H), 1.05 - 0.97 (m, 2H), 0.80 - 0.71 (m, 2H). |
| | (1S,2S)-N-(6-(((6- | |
| | cyclopropylimidazo[1,2-a]pyridin- | |
| | 2-yl)methyl)amino)pyrimidin-4- | |
| | yl)-2-(4-methylpyrimidin-2- | |
| | yl)cyclopropane-1-carboxamide | |
| | (I-381) | |
| Example 382 | 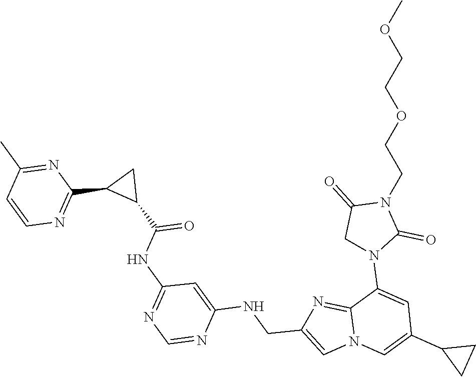 [SMILES: COCCOCCN1C(=O)CN(c2cc(C3CC3)cn3cc(CNc4cc(NC(=O)[C@H]5C[C@@H]5c5nccc(C)n5)ncn4)nc23)C1=O] | (1S,2S)-N-(6-chloropyrimidin-4-yl)-2-(4- methylpyrimidin-2-yl)cyclopropane-1- carboxamide ESI-MS (M + H)+: 641.3, ¹H NMR (400 MHz, DMSO) δ 10.69 (s, 1H), 8.52 (d, J = 5.1 Hz, 1H), 8.42 (s, 1H), 8.25 (d, J = 1.1 Hz, 1H), 8.19 (s, 1H), 7.91 (s, 1H), 7.69 (s, 1H), 7.31 (s, 2H), 7.21 (d, J = 5.2 Hz, 1H), 4.95 (s, 2H), 4.58 (s, 2H), 3.60 - 3.60 (m, 4H), 3.56 - 3.54 (m, 2H), 3.42 - 3.41 (m, 2H), 3.23 (s, 3H), 2.64 - 2.60 (m, 1H), 2.55 - 2.53 (m, 1H), 2.41 (s, 3H), 1.97 - 1.91 (m, 1H), 1.56 - 1.48 (m, 2H), 0.96 - 0.92 (m, 2H), 0.66 - 0.62 (m, 2H). |
| | (1S,2S)-N-(6-(((6-cyclopropyl-8- | |
| | (3-(2-(2-methoxyethoxy)ethyl)- | |
| | 2,4-dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-382) | |
| Example 383 | 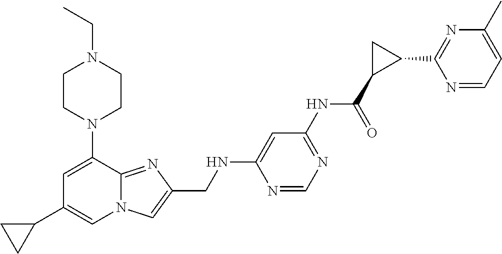 [SMILES: CCN1CCN(c2cc(C3CC3)cn3cc(CNc4cc(NC(=O)[C@H]5C[C@@H]5c5nccc(C)n5)ncn4)nc23)CC1] | (1S,2S)-N-(6-chloropyrimidin-4-yl)-2-(4- methylpyrimidin-2-yl)cyclopropane-1- carboxamide ESI-MS (M + H)+: 553.3, ¹H NMR (400 MHz, DMSO) δ 10.68 (s, 1H), 8.52 (d, J = 5.1 Hz, 1H), 8.20 - 8.16 (m, 1H), 7.89 (s, 2H), 7.54 (s, 1H), 7.27 (s, 1H), 7.21 (d, J = 5.1 Hz, 1H), 6.14 (s, 1H), 4.54 (s, 2H), 3.48 (s, 4H), 2.67 - 2.60 (m, 2H), 2.55 - 2.53 (m, 4H), 2.41 (s, 3H), 2.39 - 2.33 (m, 2H), 1.88 - 1.81 (m, 1H), 1.53 - 1.48 (m, 2H), 1.04 (t, J = 7.1 Hz, 3H), 0.88 - 0.84 (m, 2H), 0.67 - 0.58 (m, 2H). |
| | (1S,2S)-N-(6-(((6-cyclopropyl-8- | |
| | (4-ethylpiperazin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-383) | |
| Example 384 | 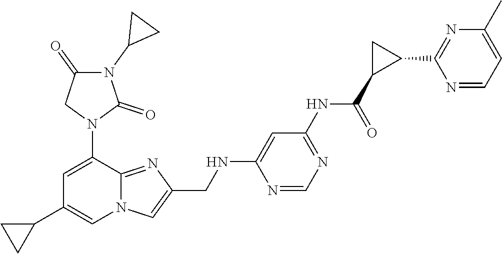 [SMILES: Cc1ccnc([C@H]2C[C@@H]2C(=O)Nc2cc(NCc3cn4cc(C5CC5)cc(N5CC(=O)N(C6CC6)C5=O)c4n3)ncn2)n1] | (1S,2S)-N-(6-chloropyrimidin-4-yl)-2-(4- methylpyrimidin-2-yl)cyclopropane-1- carboxamide ESI-MS (M + H)+: 579.2, ¹H NMR (400 MHz, DMSO) δ 10.69 (s, 1H), 8.52 (d, J = 5.1 Hz, 1H), 8.22 (d, J = 23.2 Hz, 2H), 7.90 (s, 1H), 7.69 (s, 1H), 7.31 (d, J = 9.4 Hz, 2H), 7.21 (d, J = 5.1 Hz, 1H), 4.82 (s, 2H), 4.58 (s, 2H), 2.65 - 2.59 (m, 2H), 2.55 - 2.53 (m, 1H), 2.41 (s, 3H), 1.97 - 1.90 (m, 1H), 1.56 - 1.48 (m, 2H), 0.96 - 0.85 (m, 6H), 0.66 - 0.62 (m, 2H). |
| | (1S,2S)-N-(6-(((6-cyclopropyl-8- | |
| | (3-cyclopropyl-2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-384) | |
| Example 385 | 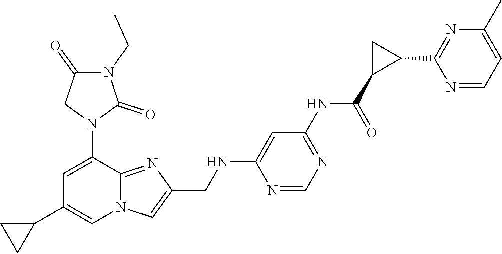 [SMILES: CCN1C(=O)CN(c2cc(C3CC3)cn3cc(CNc4cc(NC(=O)[C@H]5C[C@@H]5c5nccc(C)n5)ncn4)nc23)C1=O] | (1S,2S)-N-(6-chloropyrimidin-4-yl)-2-(4- methylpyrimidin-2-yl)cyclopropane-1- carboxamide ESI-MS (M + H)+: 567.3, ¹H NMR (400 MHz, DMSO) δ 10.70 (s, 1H), 8.55 (d, J = 5.1 Hz, 1H), 8.27 (s, 1H), 8.21 (s, 1H), 7.90 (s, 1H), 7.71 (s, 1H), 7.35 (d, J = 13.9 Hz, 2H), 7.23 (d, J = 5.1 Hz, 1H), 4.95 (s, 2H), 4.60 (s, 2H), 3.58 - 3.53 (m, 2H), 2.70 - 2.57 (m, 2H), 2.44 (s, 3H), 2.00 - 1.93 (m, 1H), 1.56 - 1.51 (m, 2H), 1.20 (t, J = 7.2 Hz, 3H), 0.99 - 0.94 (m, 2H), 0.69 - 0.65 (m, 2H). |
| | (1S,2S)-N-(6-(((6-cyclopropyl-8- | |
| | (3-ethyl-2,4-dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-385) | |
| Example 386 | 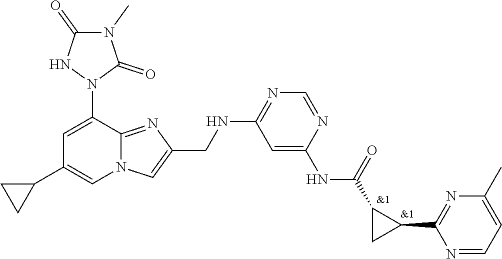 [SMILES: Cc1ccnc([C@H]2C[C@@H]2C(=O)Nc2cc(NCc3cn4cc(C5CC5)cc(-n5[nH]c(=O)n(C)c5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 554.2, ¹H NMR (400 MHz, DMSO) δ 10.84 - 10.32 (m, 2H), 8.65 - 8.46 (m, 1H), 8.32 - 8.18 (m, 1H), 8.11 - 7.66 (m, 3H), 7.34 - 7.16 (m, 2H), 6.30 - 6.12 (m, 1H), 4.62 (d, J = 29.0 Hz, 2H), 2.94 (d, J = 33.4 Hz, 3H), 2.70 - 2.57 (m, 1H), 2.51 (s, 1H), 2.43 (d, J = 16.8 Hz, 3H), 1.95 (s, 1H), 1.84 - 1.42 (m, 2H), 1.09 - 0.44 (m, 4H). |
| | rac-(1S*,2S*)-N-(6-(((6- | |
| | cyclopropyl-8-(4-methyl-3,5- | |
| | dioxo-1,2,4-triazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-386) | |
| Example 387 | 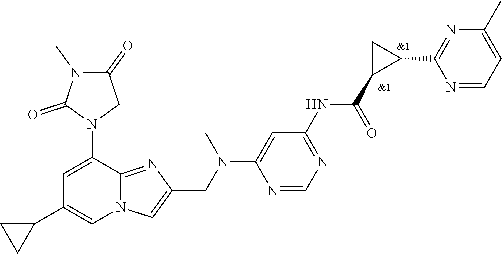 [SMILES: Cc1ccnc([C@H]2C[C@@H]2C(=O)Nc2cc(N(C)Cc3cn4cc(C5CC5)cc(N5CC(=O)N(C)C5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 567.3, ¹H NMR (400 MHz, DMSO) δ 10.78 (s, 1H), 8.52 (d, J = 5.1 Hz, 1H), 8.26 (s, 1H), 8.20 (s, 1H), 7.68 (s, 1H), 7.46 (s, 1H), 7.38 - 7.34 (m, 1H), 7.21 (d, J = 5.1 Hz, 1H), 4.90 (s, 2H), 4.81 (s, 2H), 3.13 (s, 3H), 2.97 (s, 3H), 2.64 - 2.61 (m, 1H), 2.56 - 2.53 (m, 1H), 2.41 (s, 3H), 1.97 - 1.90 (m, 1H), 1.55 - 1.50 (m, 2H), 0.96 - 0.91 (m, 2H), 0.65 - 0.61 (m, 2H) |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)(methyl)amino)pyrimidin- | |
| | 4-yl)-2-(4-methylpyrimidin-2- | |
| | yl)cyclopropane-1-carboxamide | |
| | (I-387) | |
| Example 388 | 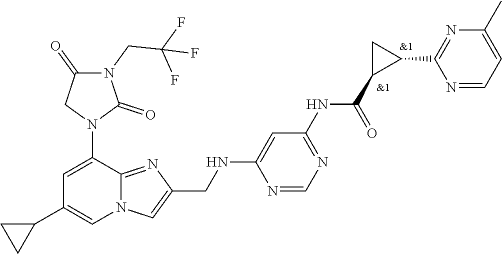 [SMILES: Cc1ccnc([C@H]2C[C@@H]2C(=O)Nc2cc(NCc3cn4cc(C5CC5)cc(N5CC(=O)N(CC(F)(F)F)C5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 621.2, ¹H NMR (400 MHz, DMSO) δ 10.68 (s, 1H), 8.52 (d, J = 5.1 Hz, 1H), 8.28 (s, 1H), 8.19 (s, 1H), 7.88 (s, 1H), 7.71 (s, 1H), 7.32 (d, J = 5.0 Hz, 2H), 7.21 (d, J = 5.1 Hz, 1H), 5.05 (s, 2H), 4.58 (s, 2H), 4.38 - 4.31 (m, 2H), 2.64 - 2.60 (m, 1H), 2.57 - 2.52 (m, 1H), 2.41 (s, 3H), 1.99 - 1.92 (m, 1H), 1.56 - 1.48 (m, 2H), 0.97 - 0.90 (m, 2H), 0.67 - 0.61 (m, 2H). |
| | rac-(1S*,2S*)-N-(6-(((6- | |
| | cyclopropyl-8-(2,4-dioxo-3- | |
| | (2,2,2-trifluoroethyl)imidazolidin- | |
| | 1-yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-388) | |
| Example 389 | 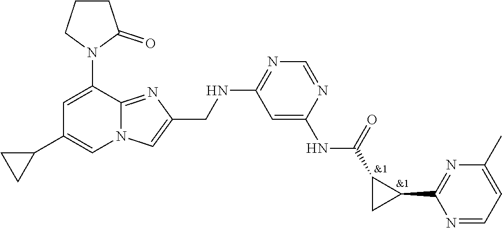 [SMILES: Cc1ccnc([C@H]2C[C@@H]2C(=O)Nc2cc(NCc3cn4cc(C5CC5)cc(N5CCCC5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 524.2, ¹H NMR (400 MHz, DMSO) δ 10.66 (s, 1H), 8.50 (d, J = 5.1 Hz, 1H), 8.22 (d, J = 1.2 Hz, 1H), 8.17 (s, 1H), 7.87 (s, 1H), 7.63 (s, 1H), 7.28 (s, 1H), 7.19 (d, J = 5.1 Hz, 1H), 7.09 (d, J = 1.3 Hz, 1H), 4.55 (s, 2H), 4.13 (t, J = 7.1 Hz, 2H), 2.62 - 2.58 (m, 1H), 2.53 - 2.52 (m, 1H), 2.46 (s, 1H), 2.44 (s, 1H), 2.40 (s, 3H), 2.14 - 2.06 (m, 2H), 1.92 - 1.87 (m, 1H), 1.54 - 1.46 (m, 2H), 0.92 - 0.87 (m, 2H), 0.64 - 0.60 (m, 2H). |
| | rac-(1S*,2S*)-N-(6-(((6- | |
| | cyclopropyl-8-(2-oxopyrrolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-389) | |
| Example 390 | 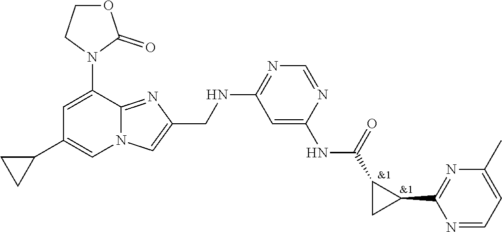 [SMILES: Cc1ccnc([C@H]2C[C@@H]2C(=O)Nc2cc(NCc3cn4cc(C5CC5)cc(N5CCOC5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 526.2, ¹H NMR (400 MHz, DMSO) δ 10.66 (s, 1H), 8.50 (d, J = 5.1 Hz, 1H), 8.23 (d, J = 1.2 Hz, 1H), 8.17 (s, 1H), 7.88 (s, 1H), 7.66 (s, 1H), 7.28 (s, 1H), 7.20 - 7.18 (m, 2H), 4.59 - 4.40 (m, 6H), 2.62 - 2.58 (m, 1H), 2.53 - 2.52 (m, 1H), 2.39 (s, 3H), 1.94 - 1.87 (m, 1H), 1.54 - 1.46 (m, 2H), 0.93 - 0.88 (m, 2H), 0.64 - 0.60 (m, 2H). |
| | rac-(1S*,2S*)-N-(6-(((6- | |
| | cyclopropyl-8-(2-oxooxazolidin- | |
| | 3-yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-390) | |
| Example 391 | 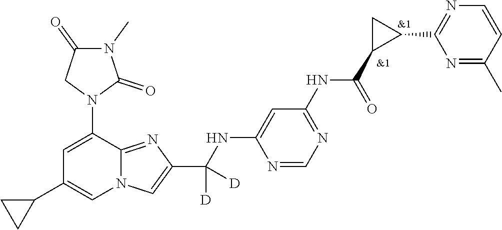 [SMILES: [2H]C([2H])(Nc1cc(NC(=O)[C@H]2C[C@@H]2c2nccc(C)n2)ncn1)c1cn2cc(C3CC3)cc(N3CC(=O)N(C)C3=O)c2n1] | rac-(1S*,2S*)-N-(6-chloropyrimidin-4-yl)-2- (4-methylpyrimidin-2-yl)cyclopropane-1- carboxamide ESI-MS (M + H)+: 555.2, ¹H NMR (400 MHz, DMSO) δ 10.67 (s, 1H), 8.52 (d, J = 5.1 Hz, 1H), 8.24 (s, 1H), 8.17 (s, 1H), 7.86 (s, 1H), 7.69 (s, 1H), 7.32 (d, J = 11.3 Hz, 2H), 7.21 (d, J = 5.1 Hz, 1H), 4.91 (s, 2H), 2.98 (s, 3H), 2.64 - 2.60 (m, 1H), 2.55 - 2.52 (m, 1H), 2.41 (s, 3H), 1.97 - 1.90 (m, 1H), 1.58 - 1.48 (m, 2H), 0.96 - 0.91 (m, 2H), 0.66 - 0.62 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl-d2)amino)pyrimidin-4- | |
| | yl)-2-(4-methylpyrimidin-2- | |
| | yl)cyclopropane-1-carboxamide | |
| | (I-391) | |
| Example 392 | 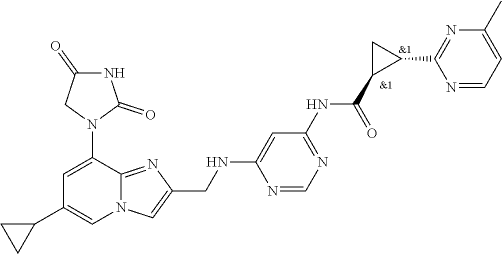 [SMILES: Cc1ccnc([C@H]2C[C@@H]2C(=O)Nc2cc(NCc3cn4cc(C5CC5)cc(N5CC(=O)NC5=O)c4n3)ncn2)n1] | rac-(1S*,2S*)-N-(6-chloropyrimidin-4-yl)-2- (4-methylpyrimidin-2-yl)cyclopropane-1- carboxamide ESI-MS (M + H)+: 539.2, ¹H NMR (400 MHz, DMSO) δ 11.33 (s, 1H), 10.68 (s, 1H), 8.52 (d, J = 5.1 Hz, 1H), 8.21 (d, J = 16.3 Hz, 2H), 7.90 (s, 1H), 7.68 (s, 1H), 7.31 (d, J = 8.4 Hz, 2H), 7.21 (d, J = 5.1 Hz, 1H), 4.87 (s, 2H), 4.58 (s, 2H), 2.72 - 2.52 (m, 2H), 2.41 (s, 3H), 2.01 - 1.85 (m, 1H), 1.55 - 1.48 (m, 2H), 1.10 - 0.84 (m, 2H), 0.79 - 0.47 (m, 2H). |
| | rac-(1S*,2S*)-N-(6-(((6- | |
| | cyclopropyl-8-(2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-392) | |
| Example 393 | 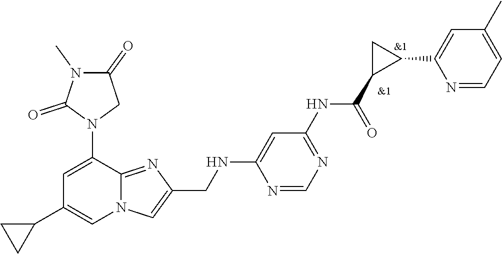 [SMILES: Cc1ccnc([C@H]2C[C@@H]2C(=O)Nc2cc(NCc3cn4cc(C5CC5)cc(N5CC(=O)N(C)C5=O)c4n3)ncn2)c1] | rac-(1S*,2S*)-N-(6-chloropyrimidin-4-yl)-2- (4-methylpyridin-2-yl)cyclopropane-1- carboxamide ESI-MS (M + H)+: 552.2, ¹H NMR (400 MHz, DMSO) δ 10.63 (s, 1H), 8.29 (d, J = 5.0 Hz, 1H), 8.25 (d, J = 1.0 Hz, 1H), 8.18 (s, 1H), 7.89 (s, 1H), 7.69 (s, 1H), 7.33 - 7.31 (m, 2H), 7.25 (s, 1H), 7.03 (d, J = 4.6 Hz, 1H), 4.91 (s, 2H), 4.58 (s, 2H), 2.98 (s, 3H), 2.47 - 2.43 (m, 2H), 2.27 (s, 3H), 1.97 - 1.90 (m, 1H), 1.52 - 1.47 (m, 1H), 1.44 - 1.40 (m, 1H), 0.96 - 0.92 (m, 2H), 0.66 - 0.62 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyridin-2- | |
| | yl)cyclopropane-1-carboxamide | |
| | (I-393) | |
| Example 394 | 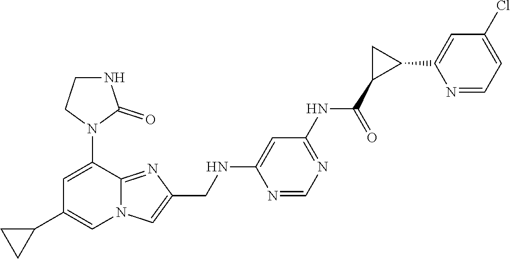 [SMILES: O=C(Nc1cc(NCc2cn3cc(C4CC4)cc(N4CCNC4=O)c3n2)ncn1)[C@H]1C[C@@H]1c1cc(Cl)ccn1] | (1S,2S)-2-(4-chloropyridin-2-yl)-N-(6- chloropyrimidin-4-yl)cyclopropane-1- carboxamide ESI-MS (M + H)+: 544.2, ¹H NMR (400 MHz, DMSO) δ 10.63 (s, 1H), 8.41 (d, J = 5.3 Hz, 1H), 8.17 (s, 1H), 8.09 (d, J = 1.1 Hz, 1H), 7.85 (s, 1H), 7.63 (d, J = 1.7 Hz, 1H), 7.60 (s, 1H), 7.33 (dd, J = 5.3, 2.0 Hz, 1H), 7.28 (s, 1H), 7.22 (s, 1H), 6.97 (s, 1H), 4.54 (s, 2H), 4.34 (t, J = 8.1 Hz, 2H), 3.42 (t, J = 8.1 Hz, 2H), 2.60 - 2.55 (m, 2H), 1.90 - 1.85 (m, 1H), 1.53 - 1.45 (m, 2H), 0.91 - 0.87 (m, 2H), 0.62 - 0.58 (m, 2H). |
| | (1S,2S)-2-(4-chloropyridin-2-yl)- | |
| | N-(6-(((6-cyclopropyl-8-(2- | |
| | oxoimidazolidin-1- | |
| | yl)imidazo[1,2-a]pyridin-2- | |
| | yl)methyl)amino)pyrimidin-4- | |
| | yl)cyclopropane-1-carboxamide | |
| | (I-394) | |
| Example 395 | 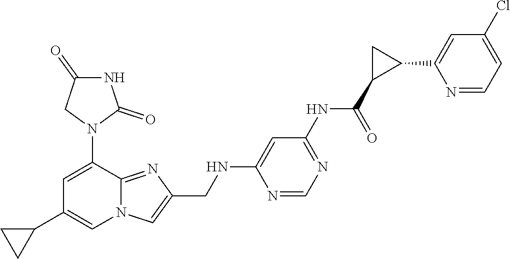 [SMILES: O=C1CN(c2cc(C3CC3)cn3cc(CNc4cc(NC(=O)[C@H]5C[C@@H]5c5cc(Cl)ccn5)ncn4)nc23)C(=O)N1] | (1S,2S)-2-(4-chloropyridin-2-yl)-N-(6- chloropyrimidin-4-yl)cyclopropane-1- carboxamide ESI-MS (M + H)+: 558.1, ¹H NMR (400 MHz, DMSO) δ 11.34 (s, 1H), 10.65 (s, 1H), 8.42 (d, J = 5.4 Hz, 1H), 8.23 - 8.18 (m, 2H), 7.89 (s, 2H), 7.68 - 7.64 (m, 2H), 7.36 - 7.31 (m, 2H), 4.88 (s, 2H), 4.57 (s, 2H), 2.62 - 2.56 (m, 2H), 1.95 - 1.91 (m, 1H), 1.53 - 1.46 (m, 2H), 0.95 - 0.91 (m, 2H), 0.66 - 0.62 (m, 2H). |
| | (1S,2S)-2-(4-chloropyridin-2-yl)- | |
| | N-(6-(((6-cyclopropyl-8-(2,4- | |
| | dioxoimidazolidin-1- | |
| | yl)imidazo[1,2-a]pyridin-2- | |
| | yl)methyl)amino)pyrimidin-4- | |
| | yl)cyclopropane-1-carboxamide | |
| | (I-395) | |
| Example 396 | 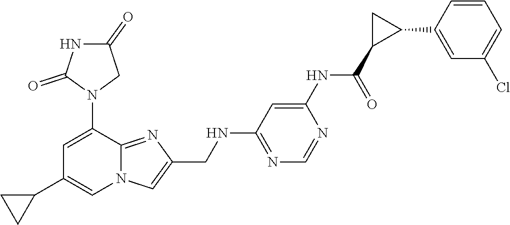 [SMILES: O=C1CN(c2cc(C3CC3)cn3cc(CNc4cc(NC(=O)[C@H]5C[C@@H]5c5cccc(Cl)c5)ncn4)nc23)C(=O)N1] | (1S,2S)-2-(3-chlorophenyl)-N-(6- chloropyrimidin-4-yl)cyclopropane-1- carboxamide ESI-MS (M + H)+: 557.2, ¹H NMR (400 MHz, MeOD) δ 8.17 (s, 1H), 8.15 (s, 1H), 7.70 (s, 1H), 7.36 (s, 1H), 7.27 - 7.24 (m, 2H), 7.20 - 7.18 (m, 2H), 7.10 (d, J = 7.6 Hz, 1H), 4.70 (s, 2H), 4.64 (s, 2H), 2.49 - 2.47 (m, 1H), 2.19 - 2.17 (m, 1H), 1.96 - 1.93 (m, 1H), 1.62 - 1.58 (m, 2H), 0.98 - 0.96 (m, 2H), 0.75 - 0.71 (m, 2H). ESI-MS [M + H]⁺: 557.2. |
| | (1S,2S)-2-(3-chlorophenyl)-N-(6- | |
| | (((6-cyclopropyl-8-(2,4- | |
| | dioxoimidazolidin-1- | |
| | yl)imidazo[1,2-a]pyridin-2- | |
| | yl)methyl)amino)pyrimidin-4- | |
| | yl)cyclopropane-1-carboxamide | |
| | (I-396) | |
| Example 397 | 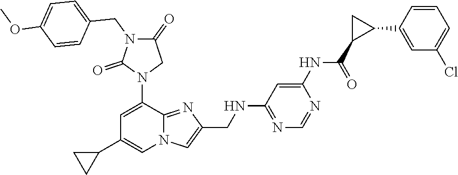 [SMILES: COc1ccc(CN2C(=O)CN(c3cc(C4CC4)cn4cc(CNc5cc(NC(=O)[C@H]6C[C@@H]6c6cccc(Cl)c6)ncn5)nc34)C2=O)cc1] | (1S,2S)-2-(3-chlorophenyl)-N-(6- chloropyrimidin-4-yl)cyclopropane-1- carboxamide ESI-MS (M + H)+: 677.2, ¹H NMR (400 MHz, DMSO) δ 10.60 (s, 1H), 8.24 (s, 1H), 8.19 (s, 1H), 7.88 (s, 1H), 7.69 (s, 1H), 7.31 - 7.25 (m, 7H), 7.15 (d, J = 7.5 Hz, 1H), 6.91 (d, J = 8.7 Hz, 2H), 4.98 (s, 2H), 4.61 - 4.55 (m, 4H), 3.73 (s, 3H), 2.42 - 2.38 (m, 2H), 1.96 - 1.92 (m, 1H), 1.48 - 1.44 (m, 1H), 1.42 - 1.39 (m, 1H), 0.95 - 0.91 (m, 2H), 0.65 - 0.62 (m, 2H). |
| | (1S,2S)-2-(3-chlorophenyl)-N-(6- | |
| | (((6-cyclopropyl-8-(3-(4- | |
| | methoxybenzyl)-2,4- | |
| | dioxoimidazolidin-1- | |
| | yl)imidazo[1,2-a]pyridin-2- | |
| | yl)methyl)amino)pyrimidin-4- | |
| | yl)cyclopropane-1-carboxamide | |
| | (I-397) | |
| Example 398 | 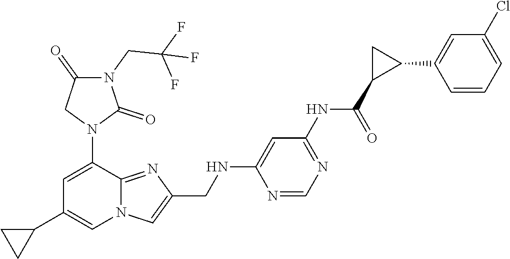 [SMILES: O=C(Nc1cc(NCc2cn3cc(C4CC4)cc(N4CC(=O)N(CC(F)(F)F)C4=O)c3n2)ncn1)[C@H]1C[C@@H]1c1cccc(Cl)c1] | (1S,2S)-2-(3-chlorophenyl)-N-(6- chloropyrimidin-4-yl)cyclopropane-1- carboxamide ESI-MS (M + H)+: 639.2, ¹H NMR (400 MHz, DMSO) δ 10.59 (s, 1H), 8.26 (s, 1H), 8.17 (s, 1H), 7.88 (s, 1H), 7.69 (s, 1H), 7.32 - 7.28 (m, 3H), 7.24 - 7.23 (m, 2H), 7.13 (d, J = 7.5 Hz, 1H), 5.03 (s, 2H), 4.56 (s, 2H), 4.36 - 4.29 (m, 2H), 2.41 - 2.34 (m, 2H), 1.99 - 1.90 (m, 1H), 1.48 - 1.43 (m, 1H), 1.41 - 1.36 (m, 1H), 0.95 - 0.90 (m, 2H), 0.65 - 0.61 (m, 2H). |
| | (1S,2S)-2-(3-chlorophenyl)-N-(6- | |
| | (((6-cyclopropyl-8-(2,4-dioxo-3- | |
| | (2,2,2-trifluoroethyl)imidazolidin- | |
| | 1-yl)imidazo[1,2-a]pyridin-2- | |
| | yl)methyl)amino)pyrimidin-4- | |
| | yl)cyclopropane-1-carboxamide | |
| | (I-398) | |
| Example 399 | 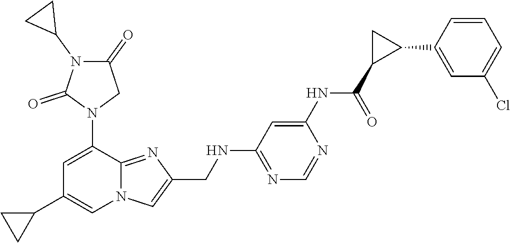 [SMILES: O=C(Nc1cc(NCc2cn3cc(C4CC4)cc(N4CC(=O)N(C5CC5)C4=O)c3n2)ncn1)[C@H]1C[C@@H]1c1cccc(Cl)c1] | (1S,2S)-2-(3-chlorophenyl)-N-(6- chloropyrimidin-4-yl)cyclopropane-1- carboxamide ESI-MS (M + H)+: 597.2, ¹H NMR (400 MHz, MeOD) δ 8.16 (s, 1H), 8.15 (d, J = 0.9 Hz, 1H), 7.69 (s, 1H), 7.36 (s, 1H), 7.27 - 7.23 (m, 2H), 7.20 - 7.18 (m, 2H), 7.10 - 7.09 (m, 1H), 4.63 (s, 2H), 4.59 (s, 2H), 2.70 - 2.64 (m, 1H), 2.50 - 2.45 (m, 1H), 2.20-2.17 (m, 1H), 1.98 - 1.91 (m, 1H), 1.62 - 1.58 (m, 1H), 1.39 - 1.35 (m, 1H), 1.02 - 0.94 (m, 6H), 0.74- 0.70 (m, 2H). ESI-MS [M + H]+: 597.2. |
| | (1S,2S)-2-(3-chlorophenyl)-N-(6- | |
| | (((6-cyclopropyl-8-(3- | |
| | cyclopropyl-2,4- | |
| | dioxoimidazolidin-1- | |
| | yl)imidazo[1,2-a]pyridin-2- | |
| | yl)methyl)amino)pyrimidin-4- | |
| | yl)cyclopropane-1-carboxamide | |
| | (I-399) | |
| Example 400 | 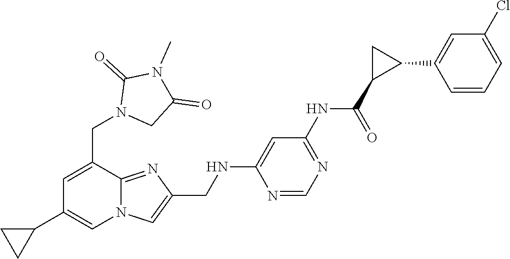 [SMILES: CN1C(=O)CN(Cc2cc(C3CC3)cn3cc(CNc4cc(NC(=O)[C@H]5C[C@@H]5c5cccc(Cl)c5)ncn4)nc23)C1=O] | (1S,2S)-2-(3-chlorophenyl)-N-(6- chloropyrimidin-4-yl)cyclopropane-1- carboxamide ESI-MS (M + H)+: 585.2, ¹H NMR (400 MHz, CDCl₃) δ 8.44 (s, 1H), 8.28 (s, 1H), 7.80 (s, 1H), 7.47 (s, 1H), 7.35 (s, 1H), 7.22 - 7.18 (m, 2H), 7.10 (s, 1H), 7.01 (d, J = 6.8 Hz, 1H), 6.95 (s, 1H), 5.82 (s, 1H), 4.86 (s, 2H), 4.66 (s, 2H), 4.11 (s, 2H), 3.04 (s, 3H), 2.60 - 2.55 (m, 1H), 1.91 - 1.83 (m, 2H), 1.42 - 1.37 (m, 2H), 0.99 - 0.94 (m, 2H), 0.68 - 0.64 (m, 2H). |
| | (1S,2S)-2-(3-chlorophenyl)-N-(6- | |
| | (((6-cyclopropyl-8-((3-methyl- | |
| | 2,4-dioxoimidazolidin-1- | |
| | yl)methyl)imidazo[1,2-a]pyridin- | |
| | 2-yl)methyl)amino)pyrimidin-4- | |
| | yl)cyclopropane-1-carboxamide | |
| | (I-400) | |
| Example 401 | 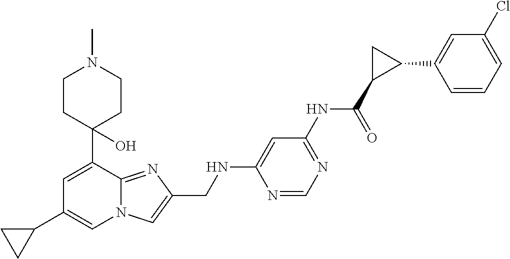 [SMILES: CN1CCC(O)(c2cc(C3CC3)cn3cc(CNc4cc(NC(=O)[C@H]5C[C@@H]5c5cccc(Cl)c5)ncn4)nc23)CC1] | (1S,2S)-2-(3-chlorophenyl)-N-(6- chloropyrimidin-4-yl)cyclopropane-1- carboxamide ESI-MS (M + H)+: 572.2, ¹H NMR (400 MHz, DMSO) δ 10.58 (s, 1H), 8.17 (s, 2H), 7.86 (s, 1H), 7.55 (s, 1H), 7.31 - 7.22 (m, 4H), 7.12 (d, J = 7.8 Hz, 1H), 7.07 - 7.06 (m, 1H), 5.37 (s, 1H), 4.54 (s, 2H), 2.87 - 2.80 (m, 2H), 2.71 (s, 2H), 2.32 - 2.30 (m, 7H), 1.92 - 1.85 (m, 1H), 1.51 - 1.43 (m, 4H), 0.92 - 0.86 (m, 2H), 0.65 - 0.60 (m, 2H). |
| | (1S,2S)-2-(3-chlorophenyl)-N-(6- | |
| | (((6-cyclopropyl-8-(4-hydroxy-1- | |
| | methylpiperidin-4-yl)imidazo[1,2- | |
| | a]pyridin-2- | |
| | yl)methyl)amino)pyrimidin-4- | |
| | yl)cyclopropane-1-carboxamide | |
| | (I-401) | |
| Example 402 | 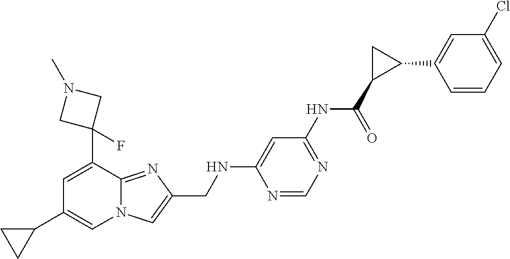 [SMILES: CN1CC(F)(c2cc(C3CC3)cn3cc(CNc4cc(NC(=O)[C@H]5C[C@@H]5c5cccc(Cl)c5)ncn4)nc23)C1] | (1S,2S)-2-(3-chlorophenyl)-N-(6- chloropyrimidin-4-yl)cyclopropane-1- carboxamide ESI-MS (M + H)+: 546.2, ¹H NMR (400 MHz, DMSO) δ 10.61 (s, 1H), 8.34 (s, 1H), 8.19 (s, 1H), 7.89 (s, 1H), 7.65 (s, 1H), 7.37 - 7.25 (m, 4H), 7.15 (d, J = 7.7 Hz, 1H), 7.07 (s, 1H), 4.61 (d, J = 21.0 Hz, 2H), 4.00 (dd, J = 20.7, 10.1 Hz, 2H), 3.67 (dd, J = 20.6, 10.1 Hz, 3H), 2.39 - 2.30 (m, 5H), 1.98 - 1.91 (m, 1H), 1.48 - 1.43 (m, 1H), 1.43 - 1.38 (m, 1H), 0.94 - 0.89 (m, 2H), 0.71 - 0.67 (m, 2H). |
| | (1S,2S)-2-(3-chlorophenyl)-N-(6- | |
| | (((6-cyclopropyl-8-(3-fluoro-1- | |
| | methylazetidin-3-yl)imidazo[1,2- | |
| | a]pyridin-2- | |
| | yl)methyl)amino)pyrimidin-4- | |
| | yl)cyclopropane-1-carboxamide | |
| | (I-402) | |
| Example 403 | 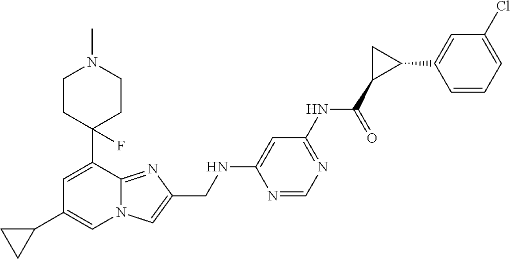 [SMILES: CN1CCC(F)(c2cc(C3CC3)cn3cc(CNc4cc(NC(=O)[C@H]5C[C@@H]5c5cccc(Cl)c5)ncn4)nc23)CC1] | (1S,2S)-2-(3-chlorophenyl)-N-(6- chloropyrimidin-4-yl)cyclopropane-1- carboxamide ESI-MS (M + H)+: 574.2, ¹H NMR (400 MHz, DMSO) δ 10.61 (s, 1H), 8.26 - 8.19 (m, 2H), 7.90 (s, 1H), 7.61 (s, 1H), 7.34 - 7.25 (m, 4H), 7.15 (d, J = 7.6 Hz, 1H), 7.00 (s, 1H), 4.57 (s, 2H), 3.16 - 3.08 (m, 1H), 3.05 - 2.97 (m, 1H), 2.77 (d, J = 10.0 Hz, 2H), 2.41 - 2.28 (m, 7H), 1.97 - 1.90 (m, 1H), 1.78 - 1.72 (m, 2H), 1.49 - 1.38 (m, 2H), 0.93 - 0.89 (m, 2H), 0.67 - 0.63 (m, 2H). |
| | (1S,2S)-2-(3-chlorophenyl)-N-(6- | |
| | (((6-cyclopropyl-8-(4-fluoro-1- | |
| | methylpiperidin-4-yl)imidazo[1,2- | |
| | a]pyridin-2- | |
| | yl)methyl)amino)pyrimidin-4- | |
| | yl)cyclopropane-1-carboxamide | |
| | (I-403) | |
| Example 404 | 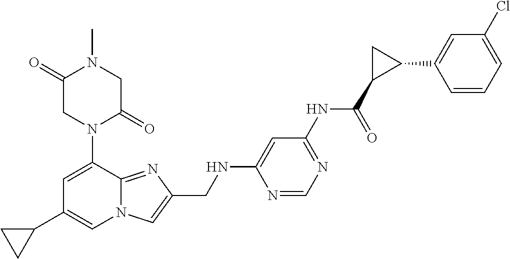 [SMILES: CN1CC(=O)N(c2cc(C3CC3)cn3cc(CNc4cc(NC(=O)[C@H]5C[C@@H]5c5cccc(Cl)c5)ncn4)nc23)CC1=O] | (1S,2S)-2-(3-chlorophenyl)-N-(6- chloropyrimidin-4-yl)cyclopropane-1- carboxamide ESI-MS (M + H)+: 585.2, ¹H NMR (400 MHz, DMSO) δ 10.60 (s, 1H), 8.33 (s, 1H), 8.19 (s, 1H), 7.92 (s, 1H), 7.67 (s, 1H), 7.39 - 7.20 (m, 4H), 7.15 (d, J = 7.6 Hz, 1H), 7.04 (d, J = 1.0 Hz, 1H), 4.56 (s, 2H), 4.42 (s, 2H), 4.20 (s, 2H), 2.93 (s, 3H), 2.47 - 2.30 (m, 2H), 1.98 - 1.87 (m, 1H), 1.51 - 1.44 (m, 1H), 1.43 - 1.37 (m, 1H), 0.98 - 0.88 (m, 2H), 0.71 - 0.61 (m, 2H). |
| | (1S,2S)-2-(3-chlorophenyl)-N-(6- | |
| | (((6-cyclopropyl-8-(4-methyl-2,5- | |
| | dioxopiperazin-1-yl)imidazo[1,2- | |
| | a]pyridin-2- | |
| | yl)methyl)amino)pyrimidin-4- | |
| | yl)cyclopropane-1-carboxamide | |
| | (I-404) | |
| Example 405 | 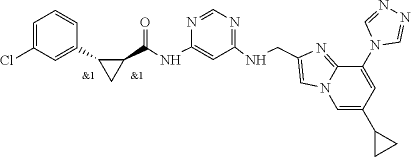 [SMILES: O=C(Nc1cc(NCc2cn3cc(C4CC4)cc(-n4cnnc4)c3n2)ncn1)[C@H]1C[C@@H]1c1cccc(Cl)c1] | (1S,2S)-2-(3-chlorophenyl)-N-(6- chloropyrimidin-4-yl)cyclopropane-1- carboxamide ESI-MS (M + H)+: 526.1, ¹H NMR (400 MHz, DMSO) δ 10.62 (s, 1H), 9.46 (s, 2H), 8.44 (s, 1H), 8.20 (s, 1H), 7.93 (s, 1H), 7.82 (s, 1H), 7.41 (s, 1H), 7.36 - 7.23 (m, 4H), 7.15 (d, J = 7.6 Hz, 1H), 4.62 (s, 2H), 2.42 - 2.37 (m,,2H), 2.12 - 1.82 (m, 1H), 1.49 - 1.41 (m, 2H), 0.99 - 0.96 (m, 2H), 0.83 - 0.80 (m, 2H). ESI-MS |
| | rac-(1S*,2S*)-2-(3-chlorophenyl)- | [M + H]⁺: 526.1. |
| | N-(6-(((6-cyclopropyl-8-(4H- | |
| | 1,2,4-triazol-4-yl)imidazo[1,2- | |
| | a]pyridin-2- | |
| | yl)methyl)amino)pyrimidin-4- | |
| | yl)cyclopropane-1-carboxamide | |
| | (I-405) | |
| Example 406 | 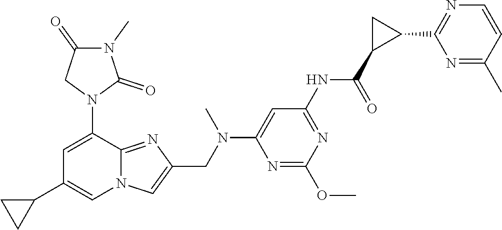 [SMILES: COc1nc(NC(=O)[C@H]2C[C@@H]2c2nccc(C)n2)cc(N(C)Cc2cn3cc(C4CC4)cc(N4CC(=O)N(C)C4=O)c3n2)n1] | (1S,2S)-N-(6-chloro-2-methoxypyrimidin-4- yl)-2-(4-methylpyrimidin-2-yl)cyclopropane- 1-carboxamide ESI-MS (M + H)+: 597.3, ¹H NMR (400 MHz, DMSO) δ 10.71 (s, 1H), 8.52 (d, J = 5.1 Hz, 1H), 8.22 (d, J = 1.0 Hz, 1H), 7.69 (s, 1H), 7.36 (d, J = 1.4 Hz, 1H), 7.20 (d, J = 5.1 Hz, 1H), 7.14 (s, 1H), 4.91 (s, 2H), 4.80 (s, 2H), 3.78 (s, 3H), 3.12 (s, 3H), 2.97 (s, 3H), 2.65 - 2.54 (m, 2H), 2.41 (s, 3H), 1.97 - 1.89 (m, 1H), 1.55 - 1.47 (m, 2H), 0.97 - 0.90 (m, 2H), 0.67 - 0.60 (m, 2H). |
| | (1S,2S)-N-(6-(((6-cyclopropyl-8- | |
| | (3-methyl-2,4-dioxoimidazolidin- | |
| | 1-yl)imidazo[1,2-a]pyridin-2- | |
| | yl)methyl)(methyl)amino)-2- | |
| | methoxypyrimidin-4-yl)-2-(4- | |
| | methylpyrimidin-2- | |
| | yl)cyclopropane-1-carboxamide | |
| | (I-406) | |
| Example 407 | 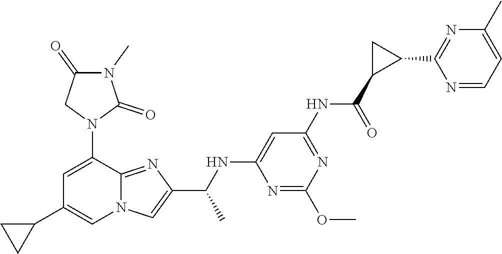 [SMILES: COc1nc(NC(=O)[C@H]2C[C@@H]2c2nccc(C)n2)cc(N[C@H](C)c2cn3cc(C4CC4)cc(N4CC(=O)N(C)C4=O)c3n2)n1] | (1S,2S)-N-(6-chloro-2-methoxypyrimidin-4- yl)-2-(4-methylpyrimidin-2-yl)cyclopropane- 1-carboxamide ESI-MS (M + H)+: 597.2, ¹H NMR (400 MHz, DMSO) δ 10.56 (s, 1H), 8.52 (d, J = 5.1 Hz, 1H), 8.23 (d, J = 1.1 Hz, 1H), 7.82 (s, 1H), 7.66 (s, 1H), 7.33 (d, J = 1.4 Hz, 1H), 7.20 (d, J = 5.1 Hz, 1H), 6.98 (s, 1H), 5.34 (s, 1H), 4.94 (s, 2H), 3.73 (s, 3H), 2.97 (s, 3H), 2.64 - 2.60 (m, 1H), 2.54 - 2.52 (m, 1H), 2.41 (s, 3H), 1.95 - 1.91 m, 1H), 1.51 - 1.49 (m, 5H), 0.96 - 0.91 (m, 2H), 0.65 - 0.61 (m, 2H). |
| | (1S,2S)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-2- | |
| | methoxypyrimidin-4-yl)-2-(4- | |
| | methylpyrimidin-2- | |
| | yl)cyclopropane-1-carboxamide | |
| | (I-407) | |
| Example 408 | 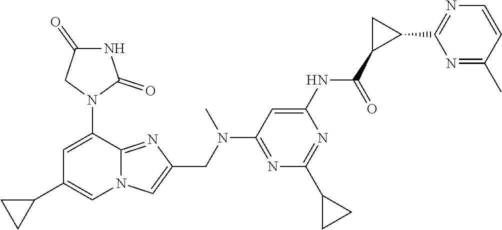 [SMILES: Cc1ccnc([C@H]2C[C@@H]2C(=O)Nc2cc(N(C)Cc3cn4cc(C5CC5)cc(N5CC(=O)NC5=O)c4n3)nc(C3CC3)n2)n1] | (1S,2S)-N-(6-chloro-2-cyclopropylpyrimidin- 4-yl)-2-(4-methylpyrimidin-2- yl)cyclopropane-1-carboxamide ESI-MS (M + H)+: 593.3, ¹H NMR (400 MHz, DMSO) δ 11.34 (s, 1H), 10.67 (s, 1H), 8.53 (d, J = 5.3 Hz, 1H), 8.22 (s, 1H), 7.63 (s, 1H), 7.36 (s, 1H), 7.22 - 7.20 (m, 2H), 4.86 (s, 2H), 4.78 (s, 2H), 3.13 (s, 3H), 2.67 - 2.64 (m, 2H), 2.41 (s, 3H), 1.95 - 1.90 (m, 1H), 1.88 - 1.82 (m, 1H), 1.53 - 1.49 (m, 2H), 1.28 - 1.23 (m, 4H), 0.86 - 0.83 (m, 2H), 0.66 - 0.62 (m, 2H). |
| | (1S,2S)-N-(2-cyclopropyl-6-(((6- | |
| | cyclopropyl-8-(2,4- | |
| | dioxoimidazolidin-1- | |
| | yl)imidazo[1,2-a]pyridin-2- | |
| | yl)methyl)(methyl)amino)pyrimidin- | |
| | 4-yl)-2-(4-methylpyrimidin-2- | |
| | yl)cyclopropane-1-carboxamide | |
| | (I-408) | |
| Example 409 | 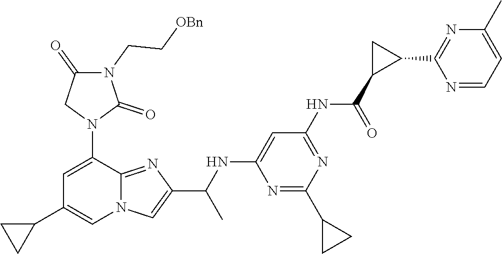 [SMILES: Cc1ccnc([C@H]2C[C@@H]2C(=O)Nc2cc(NC(C)c3cn4cc(C5CC5)cc(N5CC(=O)N(CCOCc6ccccc6)C5=O)c4n3)nc(C3CC3)n2)n1] | (1S,2S)-N-(6-chloro-2-cyclopropylpyrimidin- 4-yl)-2-(4-methylpyrimidin-2- yl)cyclopropane-1-carboxamide ESI-MS (M + H)+: 727.3, ¹H NMR (400 MHz, DMSO) δ 10.53 (s, 1H), 8.52 (d, J = 5.1 Hz, 1H), 8.26 (s, 1H), 7.70 - 7.55 (m, 2H), 7.35 - 7.27 (m, 6H), 7.20 (d, J = 5.1 Hz, 1H), 7.07 (s, 1H), 5.56 - 5.12 (m, 1H), 4.97 (s, 2H), 4.51 (s, 2H), 3.74 - 3.70 (m, 2H), 3.67 - 3.63 (m, 2H), 2.64 - 2.58 (m, 2H), 2.41 (s, 3H), 1.97 - 1.91 (m, 1H), 1.82 - 1.75 (m, 1H), 1.53 - 1.45 (m, 5H), 0.97 - 0.92 (m, 2H), 0.91 - 0.83 (m, 2H), 0.81 - 0.76 (m, 2H), 0.67 - 0.61 (m, 2H). |
| | (1S,2S)-N-(6-((1-(8-(3-(2- | |
| | (benzyloxy)ethyl)-2,4- | |
| | dioxoimidazolidin-1-yl)-6- | |
| | cyclopropylimidazo[1,2-a]pyridin- | |
| | 2-yl)ethyl)amino)-2- | |
| | cyclopropylpyrimidin-4-yl)-2-(4- | |
| | methylpyrimidin-2- | |
| | yl)cyclopropane-1-carboxamide | |
| | (I-409) | |
| Example 410 | 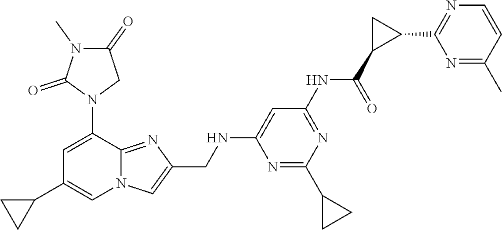 [SMILES: Cc1ccnc([C@H]2C[C@@H]2C(=O)Nc2cc(NCc3cn4cc(C5CC5)cc(N5CC(=O)N(C)C5=O)c4n3)nc(C3CC3)n2)n1] | (1S,2S)-N-(6-chloro-2-cyclopropylpyrimidin- 4-yl)-2-(4-methylpyrimidin-2- yl)cyclopropane-1-carboxamide ESI-MS (M + H)+: 593.3, ¹H NMR (400 MHz, DMSO) δ 10.55 (s, 1H), 8.52 (d, J = 5.1 Hz, 1H), 8.26 (s, 1H), 7.66 (s, 2H), 7.33 (s, 1H), 7.20 (d, J = 5.1 Hz, 1H), 7.08 (s, 1H), 4.92 (s, 2H), 4.54 (s, 2H), 2.98 (s, 3H), 2.68 - 2.56 (m, 2H), 2.41 (s, 3H), 1.94 - 1.90 (m, 1H), 1.89 - 1.71 (m, 1H), 1.60 - 1.41 (m, 2H), 1.01 - 0.75 (m, 6H), 0.73 - 0.53 (m, 2H). |
| | (1S,2S)-N-(2-cyclopropyl-6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4-yl)- | |
| | 2-(4-methylpyrimidin-2- | |
| | yl)cyclopropane-1-carboxamide | |
| | (I-410) | |
| Example 411 | 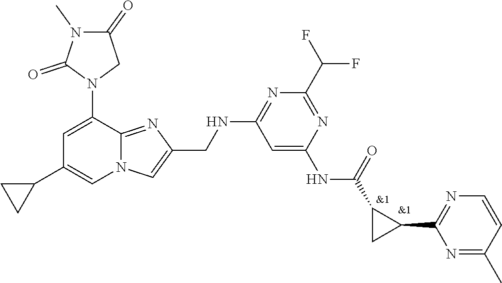 [SMILES: Cc1ccnc([C@H]2C[C@@H]2C(=O)Nc2cc(NCc3cn4cc(C5CC5)cc(N5CC(=O)N(C)C5=O)c4n3)nc(C(F)F)n2)n1] | rac-(1S*,2S*)-N-(6-chloro-2- (difluoromethyl)pyrimidin-4-yl)-2-(4- methylpyrimidin-2-yl)cyclopropane-1- carboxamide ESI-MS (M + H)+: 603.2, ¹H NMR (400 MHz, DMSO) δ 11.03 (s, 1H), 8.52 (d, J = 5.1 Hz, 1H), 8.25 (d, J = 1.1 Hz, 2H), 7.73 (s, 1H), 7.40 (s, 1H), 7.34 (s, 1H), 7.20 (d, J = 5.1 Hz, 1H), 6.54 (t, J = 54.6 Hz, 1H), 4.91 (s, 2H), 4.69 - 4.31 (m, 2H), 2.98 (s, 3H), 2.67 - 2.60 (m, 2H), 2.41 (s, 3H), 1.98 - 1.91 (m, 1H), 1.53 - 1.51 (m, 2H), 0.96 - 0.92 (m, 2H), 0.67 - 0.63 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-2- | |
| | (difluoromethyl)pyrimidin-4-yl)- | |
| | 2-(4-methylpyrimidin-2- | |
| | yl)cyclopropane-1-carboxamide | |
| | (I-411) | |
| Example 412 | 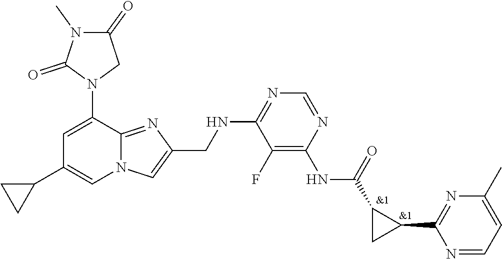 [SMILES: Cc1ccnc([C@H]2C[C@@H]2C(=O)Nc2ncnc(NCc3cn4cc(C5CC5)cc(N5CC(=O)N(C)C5=O)c4n3)c2F)n1] | rac-(1S*,2S*)-N-(6-chloro-5- fluoropyrimidin-4-yl)-2-(4-methylpyrimidin- 2-yl)cyclopropane-1-carboxamide ESI-MS (M + H)+: 571.2, ¹H NMR (400 MHz, MeOD) δ 10.57 (s, 1H), 8.54 (d, J = 5.1 Hz, 1H), 8.26-8.20 (m, 2H), 8.09 (s, 1H), 7.70 (s, 1H), 7.33 (d, J = 1.5 Hz, 1H), 7.22 (d, J = 5.1 Hz, 1H), 4.89 (s, 2H), 4.67 (d, J = 5.8 Hz, 2H), 2.97 (d, J = 2.4 Hz, 3H), 2.54 - 2.51 (m, 2H), 2.43 (s, 3H), 1.97 - 1.85 (m, 1H), 1.65 - 1.47 (m, 2H), 0.96 - 0.89 (m, 2H), 0.69 - 0.59 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-5- | |
| | fluoropyrimidin-4-yl)-2-(4- | |
| | methylpyrimidin-2- | |
| | yl)cyclopropane-1-carboxamide | |
| | (I-412) | |
| Example 413 | 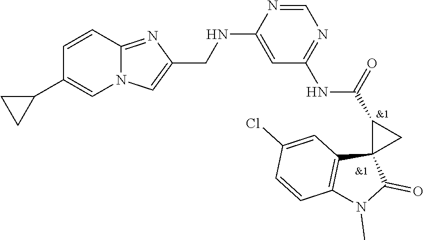 [SMILES: CN1C(=O)[C@]2(C[C@H]2C(=O)Nc2cc(NCc3cn4cc(C5CC5)ccc4n3)ncn2)c2cc(Cl)ccc21] | rac-(1R*,2S*)-5′-chloro-N-(6- chloropyrimidin-4-yl)-1′-methyl-2′- oxospiro[cyclopropane-1,3′-indoline]-2- carboxamide ESI-MS (M + H)+: 514.2, ¹H NMR (400 MHz, DMSO) δ 10.58 (s, 1H), 8.30 (s, 1H), 8.14 (s, 1H), 7.87 (s, 1H), 7.61 (s, 1H), 7.40 - 7.31 (m, 3H), 7.27 (s, 1H), 7.21 (d, J = 2.1 Hz, 1H), 7.07 (d, J = 8.3 Hz, 1H), 6.97 - 6.94 (m, 1H), 4.57 (s, 2H), 3.15 (s, 3H), 3.10 (t, J = 8.5 Hz, 1H), 2.12 - 2.07 (m, 1H), 2.00 - 1.86 (m, 3H). |
| | rac-(1R*,2S*)-5′-chloro-N-(6- | |
| | (((6-cyclopropylimidazo[1,2- | |
| | a]pyridin-2- | |
| | yl)methyl)amino)pyrimidin-4-yl)- | |
| | 1′-methyl-2′- | |
| | oxospiro[cyclopropane-1,3′- | |
| | indoline]-2-carboxamide (I-413) | |
| Example 414 | 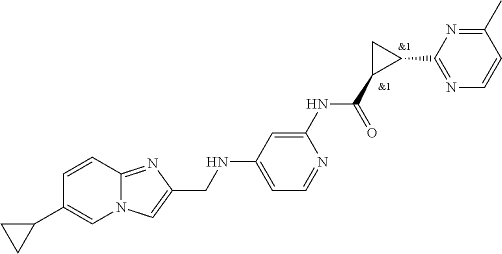 [SMILES: Cc1ccnc([C@H]2C[C@@H]2C(=O)Nc2cc(NCc3cn4cc(C5CC5)ccc4n3)ccn2)n1] | rac-(1S*,2S*)-N-(4-fluoropyridin-2-yl)-2-(4- methylpyrimidin-2-yl)cyclopropane-1- carboxamide ESI-MS (M + H)+: 440.2, ¹H NMR (400 MHz, DMSO) δ 10.47 (s, 1H), 8.51 - 8.49 (m, 1H), 8.30 (s, 1H), 7.75 - 7.74 (m, 1H), 7.63 (s, 1H), 7.38 - 7.35 (m, 2H), 7.20 - 7.18 (m, 2H), 6.98- 6.95 (m, 1H), 6.35 - 6.33 (m, 1H), 4.36 - 4.34 (m, 2H), 2.59 - 2.51 (m, 2H), 2.40 (s, 3H), 1.92 - 1.87 (m, 1H), 1.51 - 1.46 (m, 2H), 0.92 - 0.87 (m, 2H), 0.67 - 0.63 (m, 2H). |
| | rac-(1S*,2S*)-N-(4-(((6- | |
| | cyclopropylimidazo[1,2-a]pyridin- | |
| | 2-yl)methyl)amino)pyridin-2-yl)- | |
| | 2-(4-methylpyrimidin-2- | |
| | yl)cyclopropane-1-carboxamide | |
| | (I-414) | |
| Example 415 | 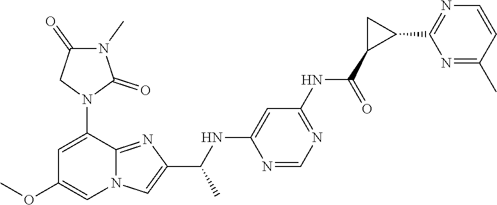 [SMILES: COc1cc(N2CC(=O)N(C)C2=O)c2nc([C@@H](C)Nc3cc(NC(=O)[C@H]4C[C@@H]4c4nccc(C)n4)ncn3)cn2c1] | (1S,2S)-N-(6-chloropyrimidin-4-yl)-2-(4- methylpyrimidin-2-yl)cyclopropane-1- carboxamide ESI-MS [M + H]+: 557.3, ¹H NMR (400 MHz, DMSO) δ 10.64 (s, 1H), 8.52 (d, J = 5.1 Hz, 1H), 8.16 (s, 1H), 8.13 (d, J = 2.1 Hz, 1H), 7.76 (s, 1H), 7.68 (s, 1H), 7.40 (d, J = 2.1 Hz, 1H), 7.31 (s, 1H), 7.20 (d, J = 5.1 Hz, 1H), 5.33 (s, 1H), 5.00 (s, 2H), 3.75 (s, 3H), 2.97 (s, 3H), 2.64 - 2.60 (m, 1H), 2.55 - 2.52 (m, 1H), 2.41 (s, 3H), 1.58 - 1.46 (m, 5H). |
| | (1S,2S)-N-(6-(((R)-1-(6-methoxy- | |
| | 8-(3-methyl-2,4- | |
| | dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-415) | |
| Example 416 | 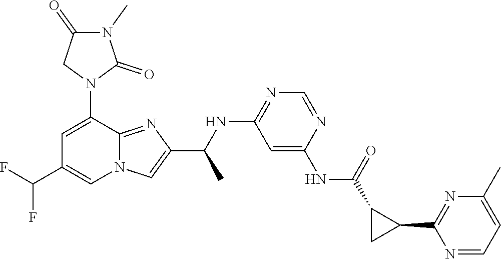 [SMILES: Cc1ccnc([C@H]2C[C@@H]2C(=O)Nc2cc(N[C@@H](C)c3cn4cc(C(F)F)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]+: 577.1. ¹H NMR (400 MHz, DMSO) δ 10.65 (s, 1H), 8.74 (s, 1H), 8.52 (d, J = 5.1 Hz, 1H), 8.16 (s, 1H), 7.91 (s, 1H), 7.82 (s, 1H), 7.77 (s, 1H), 7.32 (s, 1H), 7.26-6.99 (m, 2H), 5.47 - 5.34 (m, 1H), 5.06- 4.96 (m, 2H), 2.98 (s, 3H), 2.64 - 2.60 (m, 2H), 2.41 (s, 3H), 1.58 - 1.47 (m, 5H). |
| | (1S,2S)-N-(6-(((S)-1-(6- | |
| | (difluoromethyl)-8-(3-methyl-2,4- | |
| | dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-416) | |
| Example 417 | 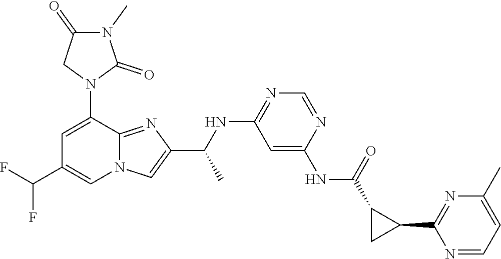 [SMILES: Cc1ccnc([C@H]2C[C@@H]2C(=O)Nc2cc(N[C@H](C)c3cn4cc(C(F)F)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]+: 577.3, ¹H NMR (400 MHz, DMSO) δ 10.66 (s, 1H), 8.74 (s, 1H), 8.52 (d, J = 5.1 Hz, 1H), 8.16 (s, 1H), 7.91 (s, 1H), 7.83 (s, 1H), 7.77 (s, 1H), 7.32 (s, 1H), 7.26-6.98 (m, 2H) ,5.45 - 5.35 (m, 1H), 5.05- 4.96 (m, 2H), 2.98 (s, 3H), 2.64 - 2.60 (m, 2H), 2.41 (s, 3H), 1.57 - 1.46 (m, 5H). |
| | (1S,2S)-N-(6-(((R)-1-(6- | |
| | (difluoromethyl)-8-(3-methyl-2,4- | |
| | dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-417) | |
| Example 418 | 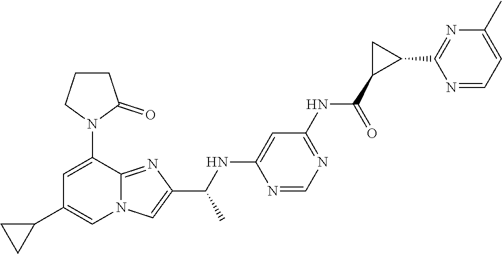 [SMILES: Cc1ccnc([C@H]2C[C@@H]2C(=O)Nc2cc(N[C@H](C)c3cn4cc(C5CC5)cc(N5CCCC5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]⁺: 538.0, ¹H NMR (400 MHz, DMSO) δ 10.65 (s, 1H), 8.52 (d, J = 5.1 Hz, 1H), 8.22 (d, J = 1.2 Hz, 1H), 8.15 (s, 1H), 7.78 (s, 1H), 7.62 (s, 1H), 7.30 (s, 1H), 7.21 (d, J = 5.1 Hz, 1H), 7.12 (d, J = 1.4 Hz, 1H), 5.32 (s, 1H), 4.17 (t, J = 7.1 Hz, 2H), 2.64 - 2.60 (m, 1H), 2.56 - 2.53 (m, 1H), 2.48 (s, 1H), 2.46 (s, 1H), 2.41 (s, 3H), 2.14 - 2.10 (m, 2H), 1.93 - 1.89 (m, 1H), 1.53 - 1.49 (m, 5H), 0.92 - 0.90 (m, 2H), 0.65 - 0.61 (m, 2H). |
| | (1S,2S)-N-(6-(((R)-1-(6- | |
| | cyclopropyl-8-(2-oxopyrrolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-418) | |
| Example 419 | 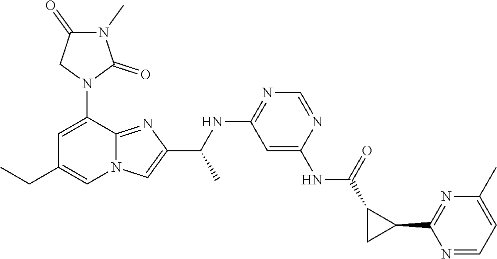 [SMILES: CCc1cc(N2CC(=O)N(C)C2=O)c2nc([C@@H](C)Nc3cc(NC(=O)[C@H]4C[C@@H]4c4nccc(C)n4)ncn3)cn2c1] | (1S,2S)-N-(6-chloropyrimidin-4-yl)-2-(4- methylpyrimidin-2-yl)cyclopropane-1- carboxamide ESI-MS [M + H]+: 555.2, ¹H NMR (400 MHz, DMSO) δ 10.64 (s, 1H), 8.52 (d, J = 5.1 Hz, 1H), 8.23 (s, 1H), 8.16 (d, J = 0.7 Hz, 1H), 7.77 (s, 1H), 7.69 (s, 1H), 7.46 (d, J = 1.4 Hz, 1H), 7.30 (s, 1H), 7.20 (d, J = 5.1 Hz, 1H), 5.33 (s, 1H), 4.95 (d, J = 1.2 Hz, 2H), 2.98 (s, 3H), 2.64 - 2.52 (m, 4H), 2.41 (s, 3H), 1.57 - 1.46 (m, 5H), 1.19 (t, J = 7.5 Hz, 3H). |
| | (1S,2S)-N-(6-(((R)-1-(6-ethyl-8- | |
| | (3-methyl-2,4-dioxoimidazolidin- | |
| | 1-yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-419) | |
| Example 420 | 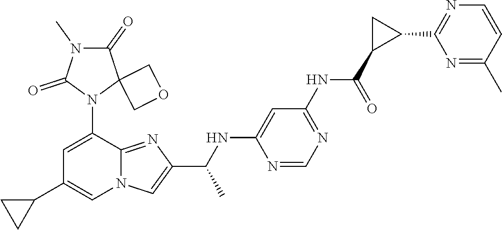 [SMILES: Cc1ccnc([C@H]2C[C@@H]2C(=O)Nc2cc(N[C@H](C)c3cn4cc(C5CC5)cc(N5C(=O)N(C)C(=O)C56COC6)c4n3)ncn2)n1] | (1S,2S)-N-(6-chloropyrimidin-4-yl)-2-(4- methylpyrimidin-2-yl)cyclopropane-1- carboxamide ESI-MS [M + H]⁺: 609.2, ¹H NMR (400 MHz, DMSO) δ 10.64 (s, 1H), 8.52 (d, J = 5.1 Hz, 1H), 8.48 (s, 1H), 8.14 (s, 1H), 7.78 (s, 1H), 7.73 (d, J = 2.2 Hz, 1H), 7.28 (s, 1H), 7.25 - 7.17 (m, 2H), 5.32 (s, 1H), 4.86 - 4.73 (m, 4H), 2.98 (s, 3H), 2.60 (d, J = 8.6 Hz, 2H), 2.41 (s, 3H), 2.04 - 1.83 (m, 1H), 1.52 - 1.46 (m, 5H), 0.97 - 0.93 (m, 2H), 0.69 - 0.65 (m, 2H). |
| | (1S,2S)-N-(6-(((R)-1-(6- | |
| | cyclopropyl-8-(7-methyl-6,8- | |
| | dioxo-2-oxa-5,7- | |
| | diazaspiro[3.4]octan-5- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-420) | |
| Example 421 | 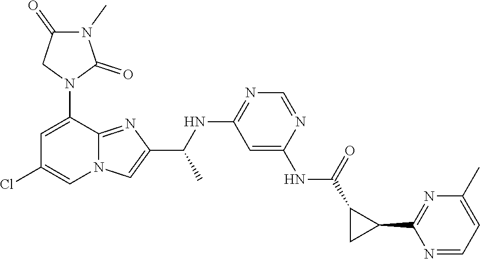 [SMILES: Cc1ccnc([C@H]2C[C@@H]2C(=O)Nc2cc(N[C@H](C)c3cn4cc(Cl)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]+: 561.1, ¹H NMR (400 MHz, DMSO) δ 10.67 (s, 1H), 8.66 (d, J = 1.9 Hz, 1H), 8.52 (d, J = 5.1 Hz, 1H), 8.16 (s, 1H), 7.84 (s, 1H), 7.77 (s, 1H), 7.67 (d, J = 1.9 Hz, 1H), 7.32 (s, 1H), 7.21 (d, J = 5.1 Hz, 1H), 5.38 (s, 1H), 5.01 (s, 1H), 2.98 (s, 3H), 2.63 - 2.54 (m, 2H), 2.41 (s, 3H), 1.52 (d, J = 6.8 Hz, 5H). |
| | (1S,2S)-N-(6-(((R)-1-(6-chloro-8- | |
| | (3-methyl-2,4-dioxoimidazolidin- | |
| | 1-yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-421) | |
| Example 422 | 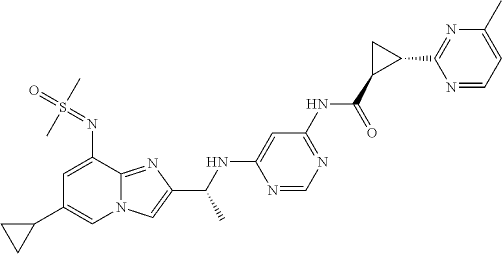 [SMILES: Cc1ccnc([C@H]2C[C@@H]2C(=O)Nc2cc(N[C@H](C)c3cn4cc(C5CC5)cc(N=S(C)(C)=O)c4n3)ncn2)n1] | (1S,2S)-N-(6-chloropyrimidin-4-yl)-2-(4- methylpyrimidin-2-yl)cyclopropane-1- carboxamide ESI-MS [M + H]⁺: 546.2, ¹H NMR (400 MHz, DMSO) δ 10.63 (s, 1H), 8.52 (d, J = 5.1 Hz, 1H), 8.15 (s, 1H), 7.84 (s, 1H), 7.74 (s, 1H), 7.49 (s, 1H), 7.26 (s, 1H), 7.21 (d, J = 5.1 Hz, 1H), 6.43 (s, 1H), 5.31 (s, 1H), 3.36 (s, 6H), 2.63 - 2.59 (m, 2H), 2.41 (s, 3H), 1.86 - 1.80 (m, 1H), 1.53 - 1.48 (m, 5H), 0.88 - 0.83 (m, 2H), 0.61 - 0.57 (m, 2H). |
| | (1S,2S)-N-(6-(((R)-1-(6- | |
| | cyclopropyl-8-((dimethyl(oxo)-16- | |
| | sulfaneylidene)amino)imidazo[1,2- | |
| | a]pyridin-2- | |
| | yl)ethyl)amino)pyrimidin-4-yl)-2- | |
| | (4-methylpyrimidin-2- | |
| | yl)cyclopropane-1-carboxamide | |
| | (I-422) | |
| Example 423 | 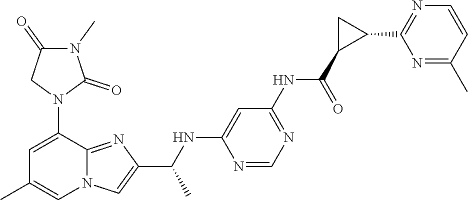 [SMILES: Cc1cc(N2CC(=O)N(C)C2=O)c2nc([C@@H](C)Nc3cc(NC(=O)[C@H]4C[C@@H]4c4nccc(C)n4)ncn3)cn2c1] | (1S,2S)-N-(6-chloropyrimidin-4-yl)-2-(4- methylpyrimidin-2-yl)cyclopropane-1- carboxamide ESI-MS [M + H]⁺: 541.3, ¹H NMR ¹H NMR (400 MHz, DMSO) δ 10.64 (s, 1H), 8.52 (d, J = 5.1 Hz, 1H), 8.21 (s, 1H), 8.16 (s, 1H), 7.76 (s, 1H), 7.69 (s, 1H), 7.42 (d, J = 1.2 Hz, 1H), 7.30 (s, 1H), 7.21 (d, J = 5.1 Hz, 1H), 5.34 (s, 1H), 4.95 (d, J = 1.1 Hz, 2H), 2.98 (s, 3H), 2.64 - 2.60 (m, 1H), 2.55 - 2.54 (m, 1H), 2.41 (s, 3H), 2.26 (s, 3H), 1.55 - 1.49 (m, 5H). |
| | (1S,2S)-N-(6-(((R)-1-(6-methyl-8- | |
| | (3-methyl-2,4-dioxoimidazolidin- | |
| | 1-yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-423) | |
| Example 424 | 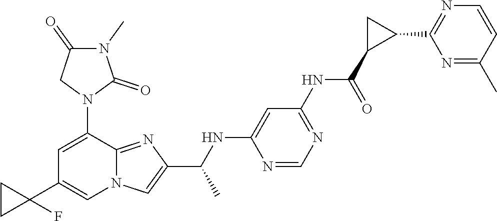 [SMILES: Cc1ccnc([C@H]2C[C@@H]2C(=O)Nc2cc(N[C@H](C)c3cn4cc(C5(F)CC5)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]+: 585.2, ¹H NMR (400 MHz, DMSO) δ 10.66 (s, 1H), 8.52 (d, J = 5.0 Hz, 2H), 8.16 (s, 1H), 7.81 (s, 1H), 7.75 (d, J = 2.3 Hz, 1H), 7.60 (d, J = 1.4 Hz, 1H), 7.31 (s, 1H), 7.21 (d, J = 5.1 Hz, 1H), 5.37 (s, 1H), 5.02 - 4.93 (m, 2H), 2.98 (s, 3H), 2.63 - 2.60 (m, 1H), 2.57 - 2.55 (m, 1H), 2.41 (s, 3H), 1.53 - 1.42 (m, 7H), 1.14 - 1.08 (m, 2H). |
| | (1S,2S)-N-(6-(((R)-1-(6-(1- | |
| | fluorocyclopropyl)-8-(3-methyl- | |
| | 2,4-dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-424) | |
| Example 425 | 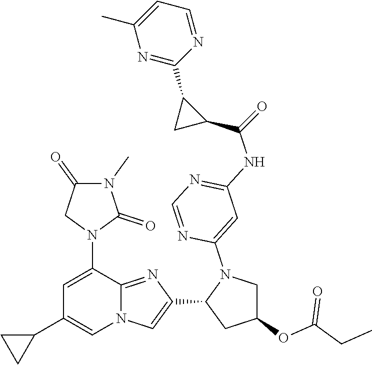 [SMILES: CCC(=O)O[C@H]1C[C@H](c2cn3cc(C4CC4)cc(N4CC(=O)N(C)C4=O)c3n2)N(c2cc(NC(=O)[C@H]3C[C@@H]3c3nccc(C)n3)ncn2)C1] | (1S,2S)-N-(6-chloropyrimidin-4-yl)-2-(4- methylpyrimidin-2-yl)cyclopropane-1- carboxamide ESI-MS [M + H]⁺: 665.3, ¹H NMR (400 MHz, DMSO) δ 10.70 (s, 1H), 8.52 (d, J = 5.1 Hz, 1H), 8.21 (d, J = 9.4 Hz, 2H), 7.76 (s, 1H), 7.37 (s, 1H), 7.29 (s, 1H), 7.20 (d, J = 5.1 Hz, 1H), 5.44 (s, 1H), 4.94 (s, 2H), 3.97 (s, 1H), 3.40-3.39 (m, 1H), 3.30 - 3.26 (m, 1H), 2.97 (s, 3H), 2.53-2.51 (m, 4H), 2.41 (s, 3H), 2.34 - 2.28 (m, 2H), 1.94-1.93 (m, 1H), 1.50-1.48 (m, 2H), 1.02 (t, J = 7.5 Hz, 3H), 0.93-0.92 (m, 2H), 0.64-0.63 (m, 2H). |
| | (3S,5R)-5-(6-cyclopropyl-8-(3- | |
| | methyl-2,4-dioxoimidazolidin-1- | |
| | yl)imidazo[1,2-a]pyridin-2-yl)-1- | |
| | (6-((1S,2S)-2-(4-methylpyrimidin- | |
| | 2-yl)cyclopropane-1- | |
| | carboxamido)pyrimidin-4- | |
| | yl)pyrrolidin-3-yl propionate (I- | |
| | 425) | |
| Example 426 | 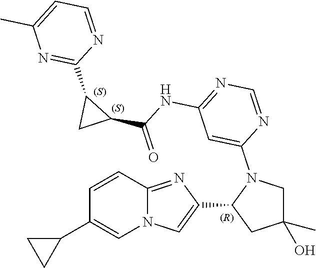 | (1S,2S)-N-(6-chloropyrimidin-4-yl)-2-(4- methylpyrimidin-2-yl)cyclopropane-1- carboxamide ESI-MS [M + H]⁺: 511.2. ¹H NMR (400 MHz, DMSO) δ 10.66 (s, 1H), 8.50 (d, J = 5.1 Hz, 1H), 8.38 - 8.30 (m, 2H), 8.19 - 8.15 (m, 1H), 7.71 (s, 1H), 7.36 (d, J = 9.2 Hz, 1H), 7.19 (d, J = 5.1 Hz, 1H), 7.01 - 6.98 (m, 1H), 5.47 (s, 1H), 5.01 - 4.96 (m, 1H), 3.79 - 3.61 (m, 2H), 2.65 - 2.59 (m, 2H), 2.39 (s, 3H), 2.32 - 2.30 (m, 1H), 2.12 (d, J = 13.4 Hz, 1H), 1.94 - 1.88 (m, 1H), 1.54 - 1.47 (m, 2H), 1.34 (s, 3H), 0.92 - 0.87 (m, 2H), 0.66 - 0.63 (m, 2H). |
| | (1S,2S)-N-(6-((2R)-2-(6- | |
| | cyclopropylimidazo[1,2-a]pyridin- | |
| | 2-yl)-4-hydroxy-4- | |
| | methylpyrrolidin-1-yl)pyrimidin- | |
| | 4-yl)-2-(4-methylpyrimidin-2- | |
| | yl)cyclopropane-1-carboxamide | |
| | (I-426) | |
| Example 427 | 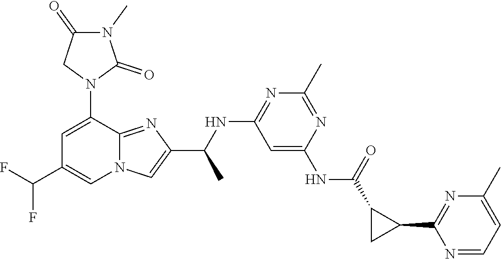 [SMILES: Cc1ccnc([C@H]2C[C@@H]2C(=O)Nc2cc(N[C@@H](C)c3cn4cc(C(F)F)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]+: 591.1. ¹H NMR (400 MHz, DMSO) δ 10.65 (s, 1H), 8.75 (s, 1H), 8.51 (d, J = 5.1 Hz, 1H), 7.92 (s, 1H), 7.77 (s, 1H), 7.70 (s, 1H), 7.26-6.99 (m, 3H), 5.52- 5.38 (m, 1H), 5.07-4.99 (m, 2H), 2.99 (s, 3H), 2.57-2.25 (m, 2H), 2.41 (s, 3H), 2.25 (s, 3H), 1.58 - 1.38 (m, 5H). |
| | (1S,2S)-N-(6-(((S)-1-(6- | |
| | (difluoromethyl)-8-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-427) | |
| Example 428 | 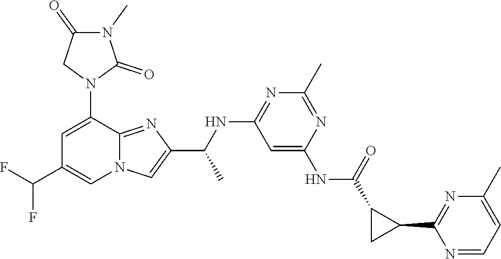 [SMILES: Cc1ccnc([C@H]2C[C@@H]2C(=O)Nc2cc(N[C@H](C)c3cn4cc(C(F)F)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]+: 591.1. ¹H NMR (400 MHz, DMSO) δ 10.64 (s, 1H), 8.75 (s, 1H), 8.51 (d, J = 5.1 Hz, 1H), 7.91 (s, 1H), 7.76 (s, 1H), 7.69 (s, 1H), 7.26-6.98 (m, 3H), 5.59 - 5.34 (m, 1H), 5.05-4.96 (m, 2H), 2.98 (s, 3H), 2.62 - 2.56 (m, 2H), 2.40 (s, 3H), 2.24 (s, 3H), 1.56 - 1.39 (m, 5H). |
| | (1S,2S)-N-(6-(((R)-1-(6- | |
| | (difluoromethyl)-8-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-428) | |
| Example 429 | 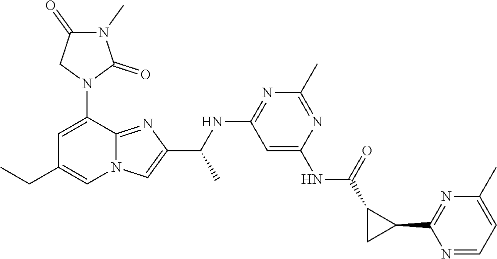 [SMILES: CCc1cc(N2CC(=O)N(C)C2=O)c2nc([C@@H](C)Nc3cc(NC(=O)[C@H]4C[C@@H]4c4nccc(C)n4)nc(C)n3)cn2c1] | (1S,2S)-N-(6-chloro-2-methylpyrimidin-4- yl)-2-(4-methylpyrimidin-2-yl)cyclopropane- 1-carboxamide ESI-MS [M + H]+: 569.2, ¹H NMR (400 MHz, DMSO) δ 10.64 (s, 1H), 8.51 (d, J = 5.1 Hz, 1H), 8.24 (s, 1H), 7.70 (s, 1H), 7.64 (d, J = 7.4 Hz, 1H), 7.47 (d, J = 1.4 Hz, 1H), 7.20 (d, J = 5.1 Hz, 1H), 7.13 (s, 1H), 5.41 (s, 1H), 5.01 - 4.91 (m, 2H), 2.98 (s, 3H), 2.61 - 2.59 (m, 4H), 2.41 (s, 3H), 2.24 (s, 3H), 1.55 - 1.46 (m, 5H), 1.19 (t, J = 7.5 Hz, 3H). |
| | (1S,2S)-N-(6-(((R)-1-(6-ethyl-8- | |
| | (3-methyl-2,4-dioxoimidazolidin- | |
| | 1-yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-429) | |
| Example 430 | 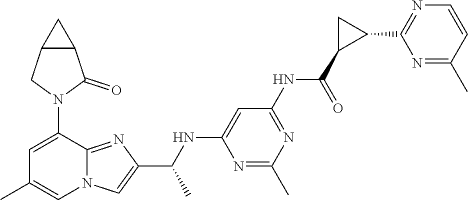 | (1S,2S)-N-(6-chloro-2-methylpyrimidin-4- yl)-2-(4-methylpyrimidin-2-yl)cyclopropane- 1-carboxamide ESI-MS [M + H]⁺: 538.3, ¹H NMR (400 MHz, DMSO) δ 10.65 (s, 1H), 8.52 (d, J = 5.1 Hz, 1H), 8.21 (s, 1H), 7.64 (s, 2H), 7.20 (d, J = 5.1 Hz, 1H), 7.11 (d, J = 9.5 Hz, 2H), 5.40 (s, 1H), 4.40-4.35 (m, 1H), 3.99 (d, J = 10.1 Hz, 1H), 2.65 - 2.53 (m, 2H), 2.41 (s, 3H), 2.23 (d, J = 10.1 Hz, 6H), 2.16 - 2.03 (m, 1H), 2.03-2.01 (m, 1H), 1.50-1.47 (m, 5H), 1.33 - 1.12 (m, 1H), 0.85-1.82 (m, 1H). |
| | (1S,2S)-N-(2-methyl-6-(((1R)-1- | |
| | (6-methyl-8-(2-oxo-3- | |
| | azabicyclo[3.1.0]hexan-3- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-430) | |
| Example 431 | 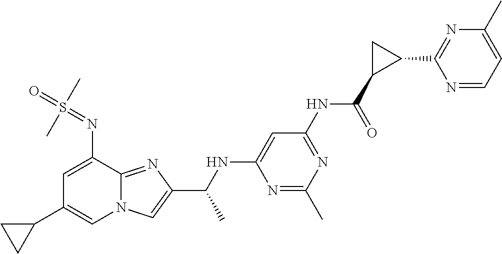 [SMILES: Cc1ccnc([C@H]2C[C@@H]2C(=O)Nc2cc(N[C@H](C)c3cn4cc(C5CC5)cc(N=S(C)(C)=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]⁺: 560.3, ¹H NMR (400 MHz, DMSO) 10.63 (s, 1H), 8.52 (d, J = 5.1 Hz, 1H), 7.85 (s, 1H), 7.60 (s, 1H), 7.48 (s, 1H), 7.20 (d, J = 5.1 Hz, 1H), 7.07 (s, 1H), 6.43 (s, 1H), 5.39 (s, 1H), 3.36 (s, 6H), 2.60 - 2.54 (m, 2H), 2.41 (s, 3H), 2.24 (s, 3H), 1.86 - 1.80 (m, 1H), 1.49 (t, J = 9.8 Hz, 5H), 0.88 - 0.81 (m, 2H), 0.59 (q, J = 5.4 Hz, 2H). |
| | (1S,2S)-N-(6-(((R)-1-(6- | |
| | cyclopropyl-8-((dimethyl(oxo)-16- | |
| | sulfaneylidene)amino)imidazo[1,2- | |
| | a]pyridin-2-yl)ethyl)amino)-2- | |
| | methylpyrimidin-4-yl)-2-(4- | |
| | methylpyrimidin-2- | |
| | yl)cyclopropane-1-carboxamide | |
| | (I-431) | |
| Example 432 | 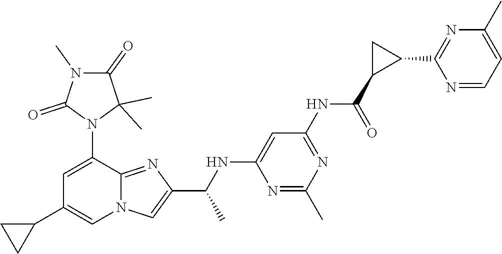 [SMILES: Cc1ccnc([C@H]2C[C@@H]2C(=O)Nc2cc(N[C@H](C)c3cn4cc(C5CC5)cc(N5C(=O)N(C)C(=O)C5(C)C)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]⁺: 609.3, ¹H NMR (400 MHz, DMSO) δ 10.64 (s, 1H), 8.52 (d, J = 5.1 Hz, 1H), 8.40 (d, J = 1.2 Hz, 1H), 7.68 - 7.59 (m, 2H), 7.20 (d, J = 5.1 Hz, 1H), 7.10 (s, 1H), 7.01 (d, J = 1.4 Hz, 1H), 5.47 - 5.27 (m, 1H), 3.00 (s, 3H), 2.63 - 2.51 (m, 2H), 2.41 (s, 3H), 2.23 (s, 3H), 1.98 - 1.91 (m, 1H), 1.57 - 1.43 (m, 5H), 1.38 - 1.35 (m, 6H), 0.95 - 0.90 (m, 2H), 0.71 - 0.64 (m, 2H). |
| | (1S,2S)-N-(6-(((R)-1-(6- | |
| | cyclopropyl-8-(3,5,5-trimethyl- | |
| | 2,4-dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-432) | |
| Example 433 | 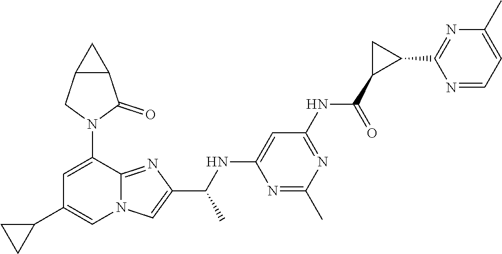 [SMILES: Cc1ccnc([C@H]2C[C@@H]2C(=O)Nc2cc(N[C@H](C)c3cn4cc(C5CC5)cc(N5CC6CC6C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]⁺: 564.2. ¹H NMR (400 MHz, DMSO) δ 10.65 (s, 1H), 8.52 (d, J = 5.1 Hz, 1H), 8.21 (d, J = 1.1 Hz, 1H), 7.70 - 7.47 (m, 2H), 7.20 (d, J = 5.2 Hz, 1H), 7.12 (s, 1H), 7.02 (s, 1H), 5.38 (s, 1H), 4.43-4.33 (m, 1H), 3.98 (d, J = 10.1 Hz, 1H), 2.65 - 2.53 (m, 2H), 2.42 (s, 3H), 2.24 (s, 3H), 2.12-2.07 (m, 1H), 2.05-1.98 (m, 1H), 1.93-1.84 (m, 1H), 1.55-1.45(m, 5H), 1.29 - 1.08 (m, 1H), 0.93- 0.83 (m, 3H),0.65-0.58 (m, 2H). |
| | (1S,2S)-N-(6-(((1R)-1-(6- | |
| | cyclopropyl-8-(2-oxo-3- | |
| | azabicyclo[3.1.0]hexan-3- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-433) | |
| Example 434 | 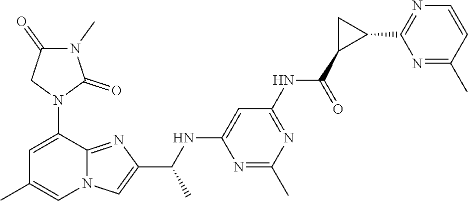 | (1S,2S)-N-(6-chloro-2-methylpyrimidin-4- yl)-2-(4-methylpyrimidin-2-yl)cyclopropane- 1-carboxamide ESI-MS [M + H]⁺: 555.2, ¹H NMR (400 MHz, DMSO) δ 10.65 (s, 1H), 8.52 (d, J = 5.1 Hz, 1H), 8.22 (s, 1H), 7.69 (s, 1H), 7.65 - 7.63 (m, 1H), 7.42 (s, 1H), 7.20 (d, J = 5.1 Hz, 1H), 7.13 (s, 1H), 5.43 (s, 1H), 5.01 - 4.91 (m, 2H), 2.98 (s, 3H), 2.61 - 2.57 (m, 1H), 2.55 - 2.53 (m, 1H), 2.41 (s, 3H), 2.26 - 2.25 (m, 6H), 1.51 - 1.46 (m, 5H). |
| | (1S,2S)-N-(2-methyl-6-(((R)-1-(6- | |
| | methyl-8-(3-methyl-2,4- | |
| | dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-434) | |
| Example 435 | 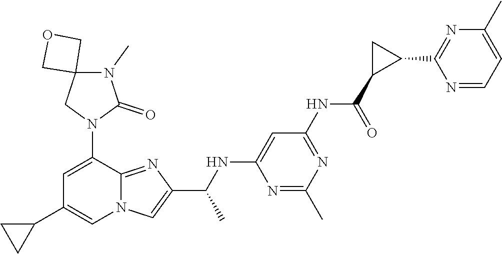 [SMILES: Cc1ccnc([C@H]2C[C@@H]2C(=O)Nc2cc(N[C@H](C)c3cn4cc(C5CC5)cc(N5CC6(COC6)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]+: 609.3, ¹H NMR (400 MHz, DMSO) δ 10.65 (s, 1H), 8.52 (d, J = 5.1, 1H), 8.12 (s, 1H), 7.59-7.52 (m, 2H), 7.23- 7.18 (m, 2H), 7.13 (s, 1H), 5.41(s, 1H), 4.98 (d, J = 7.4, 2H), 4.68 - 4.59 (m, 4H), 3.03 (s, 3H), 2.62 - 2.56 (m, 2H), 2.41 (s, 3H), 2.25 (s, 3H), 1.90-1.85 (m, 1H), 1.55-1.44 (m, 5H), 0.92-0.87 (m, 2H), 0.62-0.58 (m, 2H). |
| | (1S,2S)-N-(6-(((R)-1-(6- | |
| | cyclopropyl-8-(5-methyl-6-oxo-2- | |
| | oxa-5,7-diazaspiro[3.4]octan-7- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-435) | |
| Example 436 | 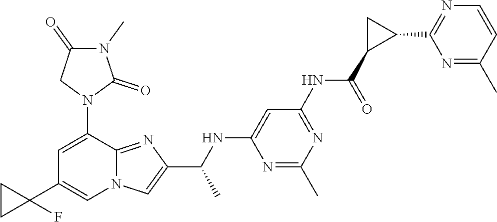 [SMILES: Cc1ccnc([C@H]2C[C@@H]2C(=O)Nc2cc(N[C@H](C)c3cn4cc(C5(F)CC5)cc(N5CC(=O)N(C)C5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]+: 599.2, ¹H NMR (400 MHz, DMSO) δ 10.66 (s, 1H), 8.54 - 8.51 (m, 2H), 7.76 (d, J = 2.6 Hz, 1H), 7.69 (d, J = 7.6 Hz, 1H), 7.60 (d, J = 1.5 Hz, 1H), 7.20 (d, J = 5.1 Hz, 1H), 7.13 (s, 1H), 5.44 (s, 1H), 5.03 - 4.93 (m, 2H), 2.98 (s, 3H), 2.64 - 2.54 (m, 2H), 2.41 (s, 3H), 2.25 (s, 3H), 1.52 - 1.43 (m, 7H), 1.14 - 1.09 (m, 2H). |
| | (1S,2S)-N-(6-(((R)-1-(6-(1- | |
| | fluorocyclopropyl)-8-(3-methyl- | |
| | 2,4-dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-436) | |
| Example 437 | 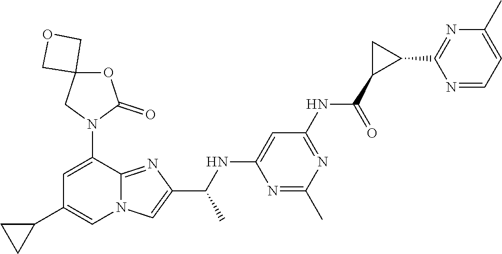 [SMILES: Cc1ccnc([C@H]2C[C@@H]2C(=O)Nc2cc(N[C@H](C)c3cn4cc(C5CC5)cc(N5CC6(COC6)OC5=O)c4n3)nc(C)n2)n1] | (1S,2S)-N-(6-chloro-2-methylpyrimidin-4- yl)-2-(4-methylpyrimidin-2-yl)cyclopropane- 1-carboxamide ESI-MS [M + H]⁺: 596.2, ¹H NMR (400 MHz, DMSO) δ 10.67 (s, 1H), 8.52 (d, J = 5.1 Hz, 1H), 8.25 (s, 1H), 7.66 (s, 2H), 7.23 - 7.13 (m, 3H), 5.48 - 5.33 (m, 1H), 4.86 - 4.75 (m, 6H), 2.61 - 2.57 (m, 2H), 2.41 (s, 3H), 2.25 (s, 3H), 1.94 - 1.91 (m, 1H), 1.52 - 1.47 (m, 5H), 0.93 - 0.91 (m, 2H), 0.64 - 0.61 (m, 2H). |
| | (1S,2S)-N-(6-(((R)-1-(6- | |
| | cyclopropyl-8-(6-oxo-2,5-dioxa-7- | |
| | azaspiro[3.4]octan-7- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-437) | |
| Example 438 | 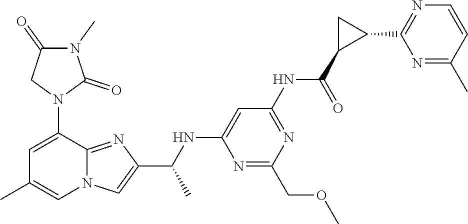 | (1S,2S)-N-(6-chloro-2-methylpyrimidin-4- yl)-2-(4-methylpyrimidin-2-yl)cyclopropane- 1-carboxamide ESI-MS [M + H]⁺: 585.2,. ¹H NMR (400 MHz, DMSO) δ 10.77 (s, 1H), 8.52 (d, J = 5.1 Hz, 1H), 8.22 (s, 1H), 7.77 (d, J = 8.1 Hz, 1H), 7.71 (s, 1H), 7.42 (d, J = 1.2 Hz, 1H), 7.20 (d, J = 5.0 Hz, 2H), 5.43 (s, 1H), 4.96 (d, J = 1.4 Hz, 2H), 4.21 (s, 2H), 3.30 (s, 3H), 2.98 (s, 3H), 2.64 - 2.59 (m, 1H), 2.56 - 2.52 (m, 1H), 2.41 (s, 3H), 2.26 (s, 3H), 1.52 - 1.48 (m, 5H). |
| | (1S,2S)-N-(2-(methoxymethyl)-6- | |
| | (((R)-1-(6-methyl-8-(3-methyl- | |
| | 2,4-dioxoimidazolidin-1- | |
| | yl)imidazo[1,2-a]pyridin-2- | |
| | yl)ethyl)amino)pyrimidin-4-yl)-2- | |
| | (4-methylpyrimidin-2- | |
| | yl)cyclopropane-1-carboxamide | |
| | (I-438) | |
| Example 439 | 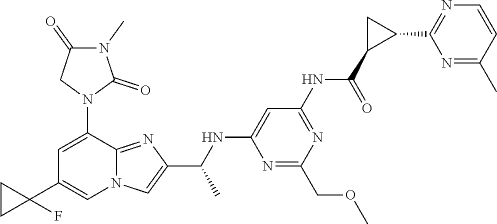 [SMILES: COCc1nc(NC(=O)[C@H]2C[C@@H]2c2nccc(C)n2)cc(N[C@H](C)c2cn3cc(C4(F)CC4)cc(N4CC(=O)N(C)C4=O)c3n2)n1] | (1S,2S)-N-(6-chloro-2-methylpyrimidin-4- yl)-2-(4-methylpyrimidin-2-yl)cyclopropane- 1-carboxamide ESI-MS [M + H]+: 629.2, ¹H NMR (400 MHz, DMSO) δ 10.79 (s, 1H), 8.54 - 8.51 (m, 2H), 7.82 (t, J = 6.9 Hz, 1H), 7.77 (s, 1H), 7.60 (d, J = 1.1 Hz, 1H), 7.20 (d, J = 5.1 Hz, 2H), 5.44 (s, 1H), 5.03 - 4.93 (m, 2H), 4.21 (s, 2H), 3.29 (s, 3H), 2.98 (s, 3H), 2.65 - 2.59 (m, 1H), 2.58 - 2.54 (m, 1H), 2.41 (s, 3H), 1.55 - 1.42 (m, 7H), 1.14 - 1.09 (m, 2H). |
| | (1S,2S)-N-(6-(((R)-1-(6-(1- | |
| | fluorocyclopropyl)-8-(3-methyl- | |
| | 2,4-dioxoimidazolidin-1- | |
| | yl)imidazo[1,2-a]pyridin-2- | |
| | yl)ethyl)amino)-2- | |
| | (methoxymethyl)pyrimidin-4-yl)- | |
| | 2-(4-methylpyrimidin-2- | |
| | yl)cyclopropane-1-carboxamide | |
| | (I-439) | |
| Example 440 | 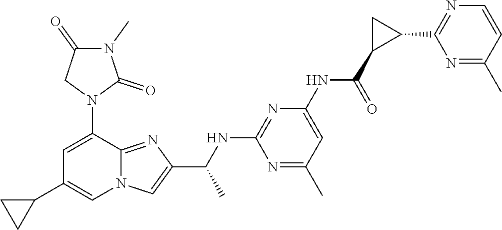 [SMILES: Cc1cc(NC(=O)[C@H]2C[C@@H]2c2nccc(C)n2)nc(N[C@H](C)c2cn3cc(C4CC4)cc(N4CC(=O)N(C)C4=O)c3n2)n1] | (1S,2S)-N-(2-chloro-6-methylpyrimidin-4- yl)-2-(4-methylpyrimidin-2-yl)cyclopropane- 1-carboxamide ESI-MS [M + H]+: 581.3, ¹H NMR (400 MHz, DMSO) δ 10.57 (s, 1H), 8.51 (d, J = 5.1 Hz, 1H), 8.21 (s, 1H), 7.65 (s, 1H), 7.31 (d, J = 1.3 Hz, 1H), 7.19 (d, J = 5.1 Hz, 1H), 7.15 (s, 1H), 5.30 - 5.27 (m, 1H), 4.91 (s, 2H), 2.96 (s, 3H), 2.67 - 2.62 (m, 1H), 2.52 - 2.52 (m, 1H), 2.40 (s, 3H), 2.21 (s, 3H), 1.93 - 1.89 (m, 1H), 1.55 - 1.47 (m, 5H), 0.94 - 0.89 (m, 2H), 0.64 - 0.60 (m, 2H). |
| | (1S,2S)-N-(2-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-6- | |
| | methylpyrimidin-4-yl)-2-(4- | |
| | methylpyrimidin-2- | |
| | yl)cyclopropane-1-carboxamide | |
| | (I-440) | |
| Example 441 | 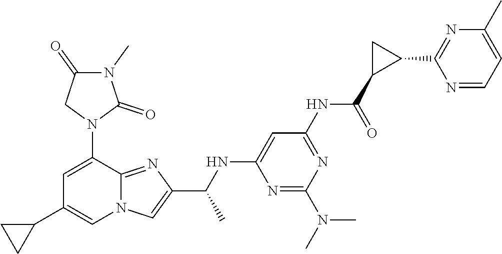 [SMILES: Cc1ccnc([C@H]2C[C@@H]2C(=O)Nc2cc(N[C@H](C)c3cn4cc(C5CC5)cc(N5CC(=O)N(C)C5=O)c4n3)nc(N(C)C)n2)n1] | (1S,2S)-N-(6-chloro-2- (dimethylamino)pyrimidin-4-yl)-2-(4- methylpyrimidin-2-yl)cyclopropane-1- carboxamide ESI-MS [M + H]⁺: 610.3, ¹H NMR (400 MHz, DMSO) δ 10.13 (s, 1H), 8.52 (d, J = 5.1 Hz, 1H), 8.24 (s, 1H), 7.64 (s, 1H), 7.32 - 7.19 (m, 3H), 6.60 (s, 1H), 5.36 - 5.29 (m, 1H), 4.93 (s, 2H), 2.98 - 2.97 (m, 9H), 2.65 - 2.59 (m, 2H), 2.41 (s, 3H), 1.95 - 1.91 (m, 1H), 1.50 - 1.45 (m, 5H), 0.95 - 0.91 (m, 2H), 0.65 - 0.62 (m, 2H). ESI-MS [M + H]⁺: 610.3. |
| | (1S,2S)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-2- | |
| | (dimethylamino)pyrimidin-4-yl)- | |
| | 2-(4-methylpyrimidin-2- | |
| | yl)cyclopropane-1-carboxamide | |
| | (I-441) | |
| Example 442 | 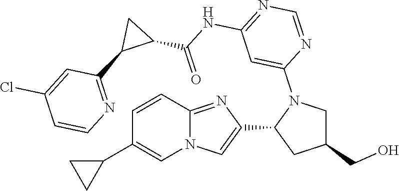 [SMILES: O=C(Nc1cc(N2C[C@@H](CO)C[C@@H]2c2cn3cc(C4CC4)ccc3n2)ncn1)[C@H]1C[C@@H]1c1cc(Cl)ccn1] | (1S,2S)-2-(4-chloropyridin-2-yl)-N-(6- chloropyrimidin-4-yl)cyclopropane-1- carboxamide ESI-MS [M + H ]⁺: 530.2, ¹H NMR (400 MHz, DMSO) δ 10.61 (s, 1H), 8.37 (d, J = 5.1 Hz, 1H), 8.21 (s, 2H), 7.57 (s, 1H), 7.43 (s, 1H), 7.35 - 7.29 (m, 3H), 6.92 (d, J = 9.2 Hz, 1H), 5.39 - 5.18 (m, 1H), 5.05 - 4.80 (m, 1H), 4.62 (s, 1H), 4.17 - 3.93 (m, 1H), 3.64 - 3.33 (m, 3H), 2.70 - 2.51 (m, 2H), 2.37 - 2.21 (m, 1H), 2.03 - 1.97 (m, 1H), 1.90 - 1.83 (m, 1H), 1.51 - 1.35 (m, 2H), 0.87 - |
| | (1S,2S)-2-(4-chloropyridin-2-yl)- | 0.85 (m, 2H), 0.62 - 0.58 (m, 2H). |
| | N-(6-((2R,4S)-2-(6- | |
| | cyclopropylimidazo[1,2-a]pyridin- | |
| | 2-yl)-4- | |
| | (hydroxymethyl)pyrrolidin-1- | |
| | yl)pyrimidin-4-yl)cyclopropane-1- | |
| | carboxamide (I-442) | |

### Examples 443-445

### Synthesis of (1S,2S)—N-(6-(((R)-1-(6-cyclopropyl-8-((1R*,5S*)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide (I-443)

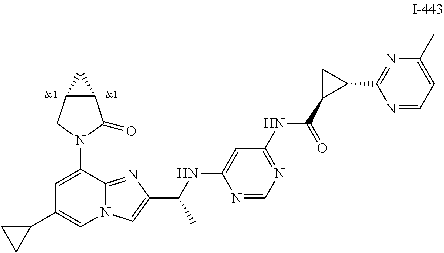 [SMILES: Cc1ccnc([C@H]2C[C@@H]2C(=O)Nc2cc(N[C@H](C)c3cn4cc(C5CC5)cc(N5C[C@@H]6C[C@@H]6C5=O)c4n3)ncn2)n1]

The title compound was prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide and obtained as a mixture of diastereomers. ESI-MS [M+H]+: 550.2, ¹H NMR (400 MHz, DMSO) δ 10.65 (s, 1H), 8.52 (d, J=5.1 Hz, 1H), 8.20 (s, 1H), 8.15 (s, 1H), 7.76 (s, 1H), 7.59 (d, J=3.0 Hz, 1H), 7.30 (s, 1H), 7.21 (d, J=5.1 Hz, 1H), 7.02 (s, 1H), 5.32 (s, 1H), 4.39-4.33 (m, 1H), 3.98 (d, J=10.4 Hz, 1H), 2.62-2.60 (m, 1H), 2.57-2.55 (m, 1H), 2.41 (s, 3H), 2.11-2.07 (m, 1H), 2.02-1.97 (m, 1H), 1.91-1.85 (m, 1H), 1.55-1.49 (m, 5H), 1.23-1.17 (m, 1H), 0.91-0.85 (m, 3H), 0.66-0.58 (m, 2H).

The mixture was separated using chiral column separation [CHIRALPAK IA, 0.46 cm I.D×15 cm L MEOH/ACN=80/20, 1 mL/min, 35° C.) to give two diastereomers: (1S,2S)—N-(6-(((R)-1-(6-cyclopropyl-8-((1S,5R)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide and (1S,2S)—N-(6-(((R)-1-(6-cyclopropyl-8-((1R,5S)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide.

First eluting isomer (I-444): ESI-MS: [M+H]+, 550.3. ¹H NMR (400 MHz, DMSO) δ 10.65 (s, 1H), 8.52 (d, J=5.1 Hz, 1H), 8.20 (d, J=1.2 Hz, 1H), 8.15 (d, J=0.7 Hz, 1H), 7.77 (s, 1H), 7.59 (s, 1H), 7.30 (s, 1H), 7.21 (d, J=5.1 Hz, 1H), 7.02 (d, J=1.3 Hz, 1H), 5.32 (s, 1H), 4.39-4.35 (m, 1H), 3.97 (d, J=10.5 Hz, 1H), 2.64-2.60 (m, 1H), 2.55-2.53 (m, 1H), 2.41 (s, 3H), 2.13-2.07 (m, 1H), 2.02-1.98 (m, 1H), 1.91-1.85 (m, 1H), 1.55-1.49 (m, 5H), 1.22-1.18 (m, 1H), 0.91-0.85 (m, 3H), 0.65-0.60 (m, 2H). RT=3.885 min, 97.4% e.e.

Second eluting isomer (I-445): ESI-MS: [M+H]+, 550.3. ¹H NMR (400 MHz, DMSO) δ 10.65 (s, 1H), 8.52 (d, J=5.0 Hz, 1H), 8.20 (s, 1H), 8.16 (s, 1H), 7.76 (s, 1H), 7.60 (s, 1H), 7.30 (s, 1H), 7.21 (d, J=5.0 Hz, 1H), 7.02 (s, 1H), 5.32 (s, 1H), 4.37-4.33 (m, 1H), 3.98 (d, J=10.4 Hz, 1H), 2.63-2.61 (m, 1H), 2.58-2.55 (m, 1H), 2.41 (s, 3H), 2.11-2.07 (m, 1H), 2.02-1.98 (m, 1H), 1.91-1.85 (m, 1H), 1.59-1.49 (m, 5H), 1.22-1.17 (m, 1H), 0.91-0.84 (m, 3H), 0.66-0.57 (m, 2H). RT=5.552 min, 99.3% e.e.

### Examples 446-448

### Synthesis of rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide (I-446)

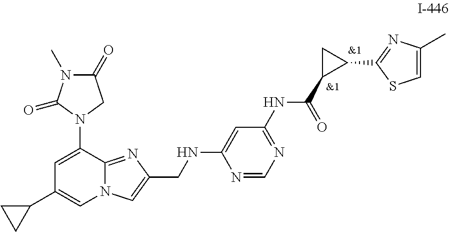 [SMILES: Cc1csc([C@H]2C[C@@H]2C(=O)Nc2cc(NCc3cn4cc(C5CC5)cc(N5CC(=O)N(C)C5=O)c4n3)ncn2)n1]

The title compound was prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide and obtained as a mixture of enantiomers. ESI-MS (M+H)+: 558.1, ¹H NMR (400 MHz, DMSO) δ 10.69 (s, 1H), 8.22 (s, 1H), 8.17 (s, 1H), 7.90 (s, 1H), 7.66 (s, 1H), ), 7.31 (s, 1H), 7.29 (s, 1H), 7.05 (s, 1H), 4.88 (s, 2H), 4.56 (s, 2H), 2.95 (s, 3H), 2.77-2.69 (m, 1H), 2.60-2.56 (m, 1H), 2.27 (s, 3H), 1.94-1.87 (m, 1H), 1.53-1.49 (m, 2H), 0.94-0.83 (m, 2H), 0.63-0.59 (m, 2H).

The mixture was separated using SFC (Daicel CHIRALPAK OZ-H 20×250 mm, 5.0 μm, 46/54 MeOH (0.2% NH₄OH)/CO₂, 45 g/min, 35° C.) to give two enantiomers: (1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide and (1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide.

First eluting isomer (I-447): ESI-MS (M+H)+: 558.2, 1 ¹H NMR (400 MHz, DMSO) δ 10.68 (s, 1H), 8.22 (s, 1H), 8.17 (s, 1H), 7.87 (s, 1H), 7.67 (s, 1H), 7.31 (s, 1H), 7.29 (s, 1H), 7.05 (s, 1H), 4.89 (s, 2H), 4.55 (s, 2H), 2.95 (s, 3H), 2.74-2.70 (m, 1H), 2.61-2.57 (m, 1H), 2.28 (s, 3H), 1.93-1.88 (m, 1H), 1.54-1.47 (m, 2H), 0.94-0.89 (m, 2H), 0.64-0.60 (m, 2H). RT=2.771 min, 100.0% e.e.

Second eluting isomer (I-448): ESI-MS (M+H)+: 558.2, ¹H NMR (400 MHz, DMSO) δ 10.68 (s, 1H), 8.22 (s, 1H), 8.17 (s, 1H), 7.88 (s, 1H), 7.67 (s, 1H), 7.31 (s, 1H), 7.28 (s, 1H), 7.05 (s, 1H), 4.89 (s, 2H), 4.55 (s, 2H), 2.95 (s, 3H), 2.74-2.70 (m, 1H), 2.61-2.58 (m, 1H), 2.28 (s, 3H), 1.93-1.88 (m, 1H), 1.53-1.47 (m, 2H), 0.94-0.89 (m, 2H), 0.64-0.60 (m, 2H). RT=5.332 min, 100.0% e.e.

### Examples 449-453

### Synthesis of rac-(1S*,2S*)—N-(6-((1-(6-cyclopropyl-8-(3-methyl-2,4-dioxoimidazolidin-1-yl) imidazo[1,2-a]pyridin-2-yl)ethyl)amino)pyrimidin-4-yl)-2-(4-methylpyrimidin-2-yl)cyclopropane-1-carboxamide (I-449)

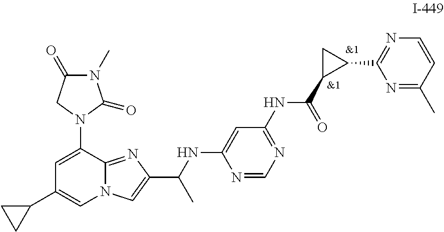 [SMILES: Cc1ccnc([C@H]2C[C@@H]2C(=O)Nc2cc(NC(C)c3cn4cc(C5CC5)cc(N5CC(=O)N(C)C5=O)c4n3)ncn2)n1]

The title compound was prepared in a similar manner to that described in Example 353 for rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide from rac-(1S*,2S*)—N-(6-chloropyrimidin-4-yl)-2-(4-methylpyrimidin-2-yl)cyclopropane-1-carboxamide and 1-(2-(1-aminoethyl)-6-cyclopropylimidazo[1,2-a]pyridin-8-yl)-3-methylimidazolidine-2,4-dione and obtained as a mixture of stereoisomers. ESI-MS (M+H)+: 567.2, ¹H NMR (400 MHz, DMSO) δ 10.65 (s, 1H), 8.52 (d, J=5.1 Hz, 1H), 8.23 (s, 1H), 8.16 (s, 1H), 7.77 (s, 1H), 7.65 (d, J=2.5 Hz, 1H), 7.34-7.30 (m, 2H), 7.21 (d, J=5.1 Hz, 1H), 5.32 (s, 1H), 4.94 (s, 2H), 2.98 (s, 3H), 2.63-2.60 (m, 1H), 2.55-2.52 (m, 1H), 2.41 (s, 3H), 1.95-1.91 (m, 1H), 1.55-1.50 (m, 5H), 0.96-0.92 (m, 2H), 0.65-0.63 (m, 2H).

The mixture was separated using SFC (Daicel CHIRALPAK AS-H 250 mm×20 mm I.D. 5 μm. CO2/MeOH (0.2% NH4OH)=52/48, 45 g/min, 35° C.) to give two separated isomers and a mixture of another two isomers. The remaining mixture was further separated using SFC (Daicel CHIRALPAK OD-H 250 mm×20 mm I.D., 5 μm. CO2/MeOH (0.2% NH4·OH)=66/34, 50 g/min, 35° C.): (1S*,2S*)—N-(6-(((R*)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, isomer 1-4 (i.e., (1S,2S)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, (1R,2R)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, (1S,2S)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide, and (1R,2R)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide).

First eluting isomer (I-450): ESI-MS [M+H]+: 567.2, ¹H NMR (400 MHz, DMSO) δ 10.65 (s, 1H), 8.52 (d, J=5.1 Hz, 1H), 8.23 (s, 1H), 8.16 (s, 1H), 7.78 (s, 1H), 7.66 (s, 1H), 7.34-7.30 (m, 2H), 7.21 (d, J=5.1 Hz, 1H), 5.33-5.31 (m, 1H), 4.94 (s, 2H), 2.98 (s, 3H), 2.64-2.59 (m, 1H), 2.57-2.54 (m, 1H), 2.41 (s, 3H), 1.94-1.91 (m, 1H), 1.54-1.49 (m, 5H), 0.95-0.92 (m, 2H), 0.65-0.62 (m, 2H). RT=2.28 min, 100% e.e.

Second eluting isomer (I-451): (1S,2S)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide. ESI-MS [M+H]+: 567.2, ¹H NMR (400 MHz, DMSO) δ 10.65 (s, 1H), 8.52 (d, J=5.1 Hz, 1H), 8.22 (s, 1H), 8.16 (s, 1H), 7.77 (s, 1H), 7.65 (s, 1H), 7.34-7.30 (m, 2H), 7.21 (d, J=5.1 Hz, 1H), 5.34-5.31 (m, 1H), 4.94 (s, 2H), 2.97 (s, 3H), 2.63-2.61 (m, 1H), 2.56-2.55 (m, 1H), 2.41 (s, 3H), 1.94-1.91 (m, 1H), 1.53-1.50 (m, 5H), 0.94-0.92 (m, 2H), 0.64-0.61 (m, 2H). RT=3.18 min, 100% e.e.

Third eluting isomer (I-452): (1S,2S)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide ESI-MS [M+H]+: 567.2, ¹H NMR (400 MHz, DMSO) δ 10.65 (s, 1H), 8.52 (d, J=5.1 Hz, 1H), 8.23 (s, 1H), 8.16 (s, 1H), 7.78 (s, 1H), 7.66 (s, 1H), 7.34-7.30 (m, 2H), 7.21 (d, J=5.1 Hz, 1H), 5.33-5.31 (m, 1H), 4.94 (s, 2H), 2.98 (s, 3H), 2.63-2.60 (m, 1H), 2.57-2.55 (m, 1H), 2.41 (s, 3H), 1.95-1.90 (m, 1H), 1.54-1.49 (m, 5H), 0.95-0.91 (m, 2H), 0.65-0.63 (m, 2H). RT=3.8 min, 100% e.e.

Fourth eluting isomer (I-453): ESI-MS [M+H]+: 567.2, ¹H NMR (400 MHz, DMSO) δ 10.66 (s, 1H), 8.52 (d, J=5.1 Hz, 1H), 8.22 (s, 1H), 8.15 (s, 1H), 7.78 (s, 1H), 7.65 (s, 1H), 7.34-7.30 (m, 2H), 7.21 (d, J=5.2 Hz, 1H), 5.34-5.31 (m, 1H), 4.94 (s, 2H), 2.97 (s, 3H), 2.62-2.60 (m, 1H), 2.56-2.54 (m, 1H), 2.41 (s, 3H), 1.95-1.91 (m, 1H), 1.54-1.50 (m, 5H), 0.95-0.92 (m, 2H), 0.65-0.61 (m, 2H). RT=5.16 min, 100% e.e.

### Example 454-456

### Synthesis of (1S,2S)—N-(6-((1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide (I-454)

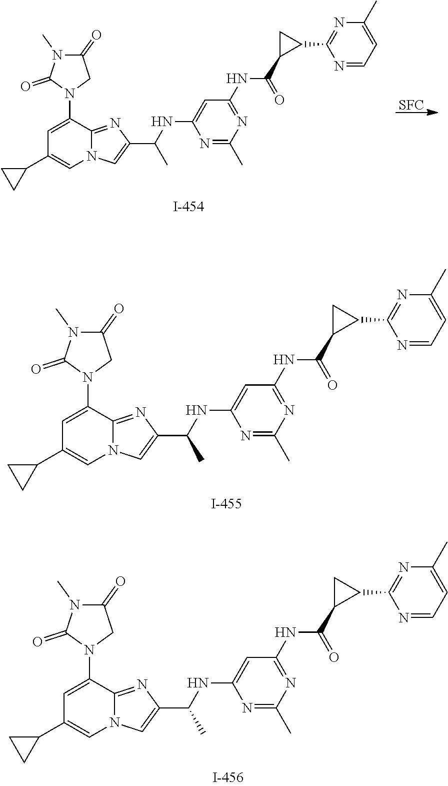

The title compound was prepared in a similar manner to that described in Example 353 for rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide from rac-(1S*,2S*)—N-(6-chloro-2-methyl-pyrimidin-4-yl)-2-(4-methylpyrimidin-2-yl)cyclopropane-1-carboxamide and 1-(2-(1-aminoethyl)-6-cyclopropylimidazo[1,2-a]pyridin-8-yl)-3-methylimidazolidine-2,4-dione and obtained as a mixture of diastereomers. ESI-MS (M+H)+: 581.2, ¹H NMR (400 MHz, DMSO) δ 10.65 (s, 1H), 8.51 (d, J=5.1 Hz, 1H), 8.23 (s, 1H), 7.65-7.63 (m, 2H), 7.33 (s, 1H), 7.20 (d, J=5.1 Hz, 1H), 7.12 (s, 1H), 5.38 (s, 1H), 4.95-4.92 (m, 2H), 2.97 (s, 3H), 2.60-2.57 (m, 2H), 2.40 (s, 3H), 2.24 (s, 3H), 1.95-1.91 (m, 1H), 1.50-1.48 (m, 5H), 0.95-0.92 (m, 2H), 0.64-0.63 (m, 2H). ESI-MS [M+H]+: 581.2.

The mixture was separated using SFC (Daicel CHIRALPAK IC-H 250 mm×20 mm I.D, 5 μm, CO₂/EtOH ((0.2% NH₄OH)=85/15, 40 g/min, UV 214 nm, 35° C.) to give two diastereomers: (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide and (1S,2S)—N-(6-(((S)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide.

First eluting isomer (I-455): (1S,2S)—N-(6S-((1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide: ESI-MS (M+H)+: 581.2, ¹H NMR (400 MHz, DMSO) δ 10.66 (s, 1H), 8.52 (d, J=5.1 Hz, 1H), 8.25 (s, 1H), 7.67-7.60 (m, 2H), 7.35 (s, 1H), 7.21 (d, J=5.1 Hz, 1H), 7.13 (s, 1H), 5.40 (s, 1H), 4.96 (d, J=4.1 Hz, 2H), 2.98 (s, 3H), 2.67-2.58 (m, 2H), 2.41 (s, 3H), 2.25 (s, 3H), 1.96-1.92 (m, 1H), 1.55-1.47 (m, 5H), 0.97-0.92 (m, 2H), 0.66-0.63 (m, 2H). RT=5.439 min, 100%, e.e

Second eluting isomer (I-456): (1S,2S)—N-(6R-((1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide. ESI-MS (M+H)+: 581.2, ¹H NMR (400 MHz, DMSO) δ 10.66 (s, 1H), 8.53 (d, J=5.1 Hz, 1H), 8.25 (d, J=1.2 Hz, 1H), 7.67-7.63 (m, 2H), 7.35 (s, 1H), 7.22 (s, 1H), 7.14 (s, 1H), 5.41 (s, 1H), 4.96 (d, J=2.7 Hz, 2H), 2.99 (s, 3H), 2.69-2.58 (m, 2H), 2.43 (s, 3H), 2.26 (s, 3H), 1.94-1.94 (m, 1H), 1.52-1.51 (m, 5H), 0.96-0.94 (m, 2H), 0.67-0.63 (m, 2H). RT=6.889 min, 100% e.e.

### Examples 457-459

### Synthesis of rac-(1S,2S)-2-(3-chlorophenyl)-N-(6-(((6-cyclopropyl-8-((1R*,5S*)-2-oxo-3-azabicyclo[3.1.0]hexan-3-yl)imidazo[1,2-a]pyridin-2-yl)methyl)amino)pyrimidin-4-yl)cyclopropane-1-carboxamide (I-457)

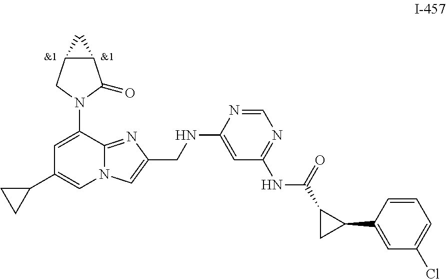 [SMILES: O=C(Nc1cc(NCc2cn3cc(C4CC4)cc(N4C[C@@H]5C[C@@H]5C4=O)c3n2)ncn1)[C@H]1C[C@@H]1c1cccc(Cl)c1]

The title compound was prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide and obtained as a mixture of diastereomers. ESI-MS (M+H)+: 554.2, ¹H NMR (400 MHz, DMSO) δ 10.60 (s, 1H), 8.20 (d, J=8.7 Hz, 2H), 7.87 (s, 1H), 7.63 (s, 1H), 7.41-7.19 (m, 4H), 7.15 (d, J=7.6 Hz, 1H), 7.03 (s, 1H), 4.55 (s, 2H), 4.35-4.29 (m, 1H), 3.98 (d, J=10.3 Hz, 1H), 2.41-2.36 (m, 2H), 2.11-2.06 (m, 1H), 2.05-1.98 (m, 1H), 1.91-1.87 (m, 1H), 1.50-1.38 (m, 2H), 1.22-1.08 (m, 1H), 0.93-0.82 (m, 3H), 0.65-0.57 (m, 2H).

The mixture was separated using chiral column separation [CHIRALPAK IC, 2.5 cm I.D×25 cm L MEOH=100, 30 mL/min, 35° C.) to give two diastereomers: (1S,2S)-2-(3-chlorophenyl)-N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)cyclopropane-1-carboxamide and (1S,2S)—N-(5-((6-cyclopropyl-8-((1S,5R)-2-oxo-3-azabicyclo[3.1.0]hexan-3-yl)imidazo[1,2-a]pyridin-2-yl)methoxy)pyridazin-3-yl)-2-(4-methylpyrimidin-2-yl)cyclopropane-1-carboxamide

First eluting isomer (I-458) ESI-MS (M+H)+: 554.2, ¹H NMR (400 MHz, DMSO) δ 10.61 (s, 1H), 8.20 (d, J=8.7 Hz, 2H), 7.89 (s, 1H), 7.63 (s, 1H), 7.41-7.23 (m, 4H), 7.15 (d, J=7.6 Hz, 1H), 7.03 (s, 1H), 4.56 (s, 2H), 4.35-4.29 (m, 1H), 3.98 (d, J=10.3 Hz, 1H), 2.32-2.39 (m, 2H), 2.12-2.06 (m, 1H), 2.05-1.98 (m, 1H), 1.94-1.85 (m, 1H), 1.50-1.38 (m, 2H), 1.23-1.17 (m, 1H), 0.92-0.83 (m, 3H), 0.64-0.62 (m, 2H). RT=6.328 min, 100.0% e.e.

Second eluting isomer (I-459): ESI-MS (M+H)+: 554.2, H NMR (400 MHz, DMSO) δ 10.60 (s, 1H), 8.20 (d, J=8.7 Hz, 2H), 7.87 (s, 1H), 7.68 (s, 1H), 7.41-7.19 (m, 4H), 7.15 (d, J=7.6 Hz, 1H), 7.03 (s, 1H), 4.55 (s, 2H), 4.36-4.29 (m, 1H), 3.98 (d, J=10.3 Hz, 1H), 2.41-2.36 (m, 2H), 2.11-2.06 (m, 1H), 2.05-1.98 (m, 1H), 1.91-1.87 (m, 1H), 1.50-1.38 (m, 2H), 1.22-1.10 (m, 1H), 0.93-0.82 (m, 3H), 0.65-0.57 (m, 2H). RT=7.635 min, 100.0% e.e.

### Example 460

### Synthesis of (1S,2S)—N-(2-cyclopropyl-6-((1-(6-cyclopropyl-8-(3-(2-hydroxyethyl)-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-460)

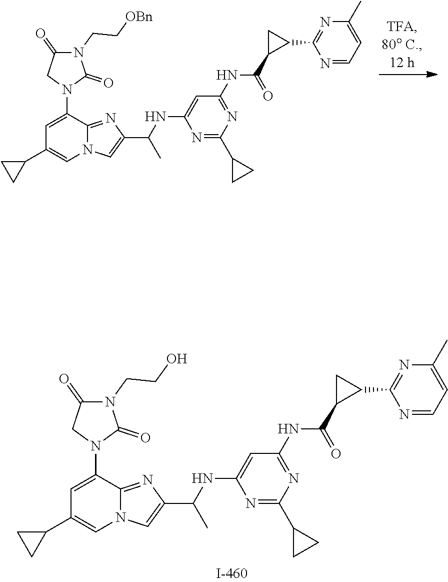

A solution of (1S,2S)—N-(6-((1-(8-(3-(2-(benzyloxy)ethyl)-2,4-dioxoimidazolidin-1-yl)-6-cyclopropylimidazo[1,2-a]pyridin-2-yl)ethyl)amino)-2-cyclopropylpyrimidin-4-yl)-2-(4-methylpyrimidin-2-yl)cyclopropane-1-carboxamide (40 mg, 0.06 mmol) in TFA (3 mL) was stirred at 80° C. for 12 h. The reaction mixture was concentrated in vacuo, diluted with NH₃/MeOH (7M, 2 mL) and stirred at room temperature for 10 min. Removal of the solvents gave the crude product, which was purified by preparative TLC (eluted with DCM:MeOH=20:1) to give (1S,2S)—N-(2-cyclopropyl-6-((1-(6-cyclopropyl-8-(3-(2-hydroxyethyl)-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide as a white solid. (22 mg, 58%). ESI-MS (M+H)+: 637.3, ¹H NMR (400 MHz, DMSO) δ 10.53 (s, 1H), 8.52 (d, J=5.1 Hz, 1H), 8.24 (s, 1H), 7.69-7.55 (m, 2H), 7.33 (s, 1H), 7.20 (d, J=5.1 Hz, 1H), 7.07 (s, 1H), 5.53-5.21 (m, 1H), 4.96 (s, 2H), 4.92-4.87 (m, 1H), 3.60-3.54 (m, J=9.5 Hz, 4H), 2.63-2.60 (m, 1H), 2.55-2.53 (m, 1H), 2.41 (s, 3H), 1.97-1.91 (m, 1H), 1.82-1.75 (m, 1H), 1.53-1.45 (m, 5H), 0.96-0.92 (m, 2H), 0.91-0.84 (m, 2H), 0.82-0.78 (m, 2H), 0.66-0.60 (m, 2H).

The compounds in Table 34 were prepared in a similar manner to (1S,2S)—N-(2-cyclopropyl-6-((1-(6-cyclopropyl-8-(3-(2-hydroxyethyl)-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide from the corresponding benzyl esters or ethers, which were in turn prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide.

TABLE 34

| Example | Structure | Analytical Data |
|---|---|---|
| Example 461 | 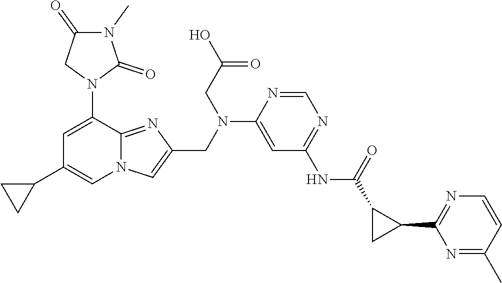 [SMILES: Cc1ccnc([C@H]2C[C@@H]2C(=O)Nc2cc(N(CC(=O)O)Cc3cn4cc(C5CC5)cc(N5CC(=O)N(C)C5=O)c4n3)ncn2)n1] | ESI-MS (M + H)⁺: 611.2, ¹H NMR (400 MHz, DMSO) δ 10.77 (s, 1H), 8.52 (d, J = 5.0 Hz, 1H), 8.25 (s, 2H), 7.87 − 7.60 (m, 2H), 7.36 (s, 1H), 7.20 (d, J = 5.0 Hz, 1H), 4.92 (s, 2H), 4.64 (s, 2H), 4.37 (s, 2H), 2.98 (s, 3H), 2.69 − 2.57 (m, 2H), 2.41 (s, 3H), 2.00 − 1.83 (m, 1H), 1.61 − 1.46 (m, 2H), 1.00 − 0.88 (m, 2H), 0.76 − 0.51 (m, 2H). |
| | N-((6-cyclopropyl-8-(3-methyl-2,4- | |
| | dioxoimidazolidin-1-yl)imidazo[1,2- | |
| | a]pyridin-2-yl)methyl)-N-(6-((1S,2S)- | |
| | 2-(4-methylpyrimidin-2- | |
| | yl)cyclopropane-1- | |
| | carboxamido)pyrimidin-4-yl)glycine | |
| | (I-461) | |
| Example 462 | 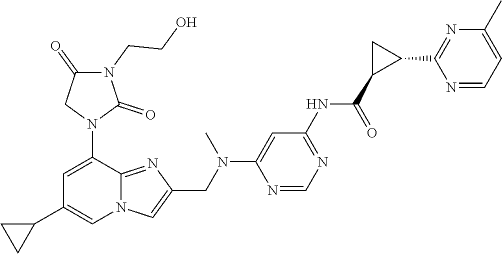 [SMILES: Cc1ccnc([C@H]2C[C@@H]2C(=O)Nc2cc(N(C)Cc3cn4cc(C5CC5)cc(N5CC(=O)N(CCO)C5=O)c4n3)ncn2)n1] | ESI-MS (M + H)+: 597.2, ¹H NMR (400 MHz, DMSO) δ 10.76 (s, 1H), 8.52 (d, J = 5.1 Hz, 1H), 8.26 (d, J = 0.6 Hz, 1H), 8.20 (s, 1H), 7.68 (s, 1H), 7.46 (s, 1H), 7.37 (d, J = 1.0 Hz, 1H), 7.20 (d, J = 5.1 Hz, 1H), 4.91 − 4.81 (m, 5H), 3.59 − 3.56 (m, 4H), 3.13 (s, 3H), 2.67 − 2.62 (m, 1H), 2.56 − 2.54 (m, 1H), 2.41 (s, 3H), 1.97 − 1.91 (m, 1H), 1.56 − 1.50 (m, 2H), 0.96 − 0.91 (m, 2H), 0.65 − 0.62 (m, 2H). |
| | (1S,2S)-N-(6-(((6-cyclopropyl-8-(3- | |
| | (2-hydroxyethyl)-2,4- | |
| | dioxoimidazolidin-1-yl)imidazo[1,2- | |
| | a]pyridin-2- | |
| | yl)methyl)(methyl)amino)pyrimidin- | |
| | 4-yl)-2-(4-methylpyrimidin-2- | |
| | yl)cyclopropane-1-carboxamide (I- | |
| | 462) | |
| Example 463 | 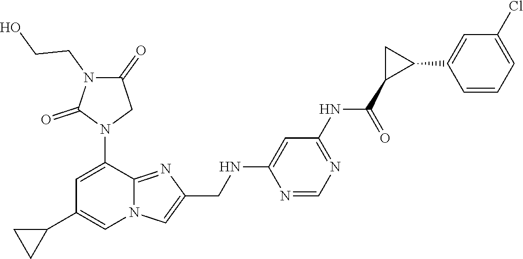 [SMILES: O=C(Nc1cc(NCc2cn3cc(C4CC4)cc(N4CC(=O)N(CCO)C4=O)c3n2)ncn1)[C@H]1C[C@@H]1c1cccc(Cl)c1] | ESI-MS (M + H)+: 601.2, ¹H NMR (400 MHz, MeOD) δ 8.17 (s, 2H), 7.71 (s, 1H), 7.36 (s, 1H), 7.32 − 7.23 (m, 2H), 7.22 − 7.16 (m,, 2H), 7.10 (d, J = 7.6 Hz, 1H), 4.69 (s, 2H), 4.64 (s, 2H), 3.83 − 3.70 (m, 4H), 2.51 − 2.45 (m, 1H), 2.21 − 2.15 (m, 1H), 1.98 − 1.93 (m, 1H), 1.64 − 1.57 (m, 1H), 1.41 − 1.35 (m, 1H), 1.02 − 0.95 (m, 2H), 0.78 − 0.70 (m, 2H). |
| | (1S,2S)-2-(3-chlorophenyl)-N-(6-(((6- | |
| | cyclopropyl-8-(3-(2-hydroxyethyl)- | |
| | 2,4-dioxoimidazolidin-1- | |
| | yl)imidazo[1,2-a]pyridin-2- | |
| | yl)methyl)amino)pyrimidin-4- | |
| | yl)cyclopropane-1-carboxamide (I- | |
| | 463) | |
| Example 464 | 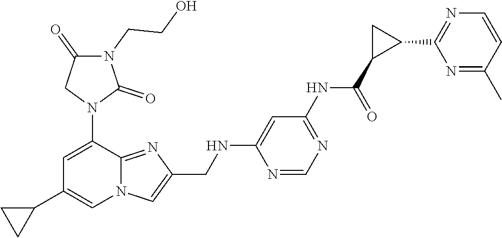 [SMILES: Cc1ccnc([C@H]2C[C@@H]2C(=O)Nc2cc(NCc3cn4cc(C5CC5)cc(N5CC(=O)N(CCO)C5=O)c4n3)ncn2)n1] | ESI-MS (M + H)+: 583.2, ¹H NMR (400 MHz, DMSO) δ 10.67 (s, 1H), 8.52 (d, J = 5.1 Hz, 1H), 8.24 (d, J = 1.0 Hz, 1H), 8.18 (s, 1H), 7.89 (s, 1H), 7.69 (s, 1H), 7.31 (d, J = 10.6 Hz, 2H), 7.20 (d, J = 5.1 Hz, 1H), 4.93 (s, 2H), 4.89 (s, 1H), 4.57 (s, 2H), 3.61 − 3.53 (m, 4H), 2.64 − 2.59 (m, 1H), 2.53 − 2.51 (m, 1H), 2.41 (s, 3H), 1.97 − 1.90 (m, 1H), 1.55 − 1.47 (m, 2H), 0.96 − 0.91 (m, 2H), 0.66 − 0.62 (m, 2H). |
| | (1S,2S)-N-(6-(((6-cyclopropyl-8-(3- | |
| | (2-hydroxyethyl)-2,4- | |
| | dioxoimidazolidin-1-yl)imidazo[1,2- | |
| | a]pyridin-2- | |
| | yl)methyl)amino)pyrimidin-4-yl)-2- | |
| | (4-methylpyrimidin-2- | |
| | yl)cyclopropane-1-carboxamide (I- | |
| | 464) | |

### Example 465

### Synthesis of 2-(3-(6-cyclopropyl-2-(((6-((1S,2S)-2-(4-methylpyrimidin-2-yl)cyclopropane-1-yl)acetic acid. (I-465)

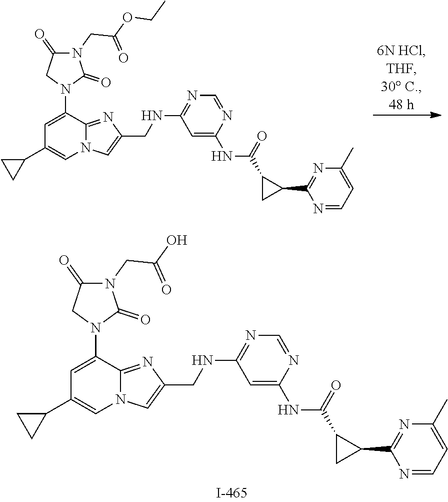 [SMILES: CCOC(=O)CN1C(=O)CN(c2cc(C3CC3)cn3cc(CNc4cc(NC(=O)[C@H]5C[C@@H]5c5nccc(C)n5)ncn4)nc23)C1=O ; Cc1ccnc([C@H]2C[C@@H]2C(=O)Nc2cc(NCc3cn4cc(C5CC5)cc(N5CC(=O)N(CC(=O)O)C5=O)c4n3)ncn2)n1]

To a solution of ethyl 2-(3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)acetate (30 mg, 0.05 mmol) in THF (1 mL) was added 6 N HCl (0.5 mL) at room temperature. The reaction mixture was stirred at 35° C. for 48 h and then concentrated in vacuo to provide crude product which was purified by preparative HPLC to afford product (2 mg, 7% yield) as a white solid. ESI-MS (M+H)+: 597.2, ¹H NMR (400 MHz, DMSO) δ 10.67 (s, 1H), 8.52 (d, J=5.0 Hz, 1H), 8.24 (s, 1H), 8.19 (s, 1H), 7.93 (s, 1H), 7.69 (s, 1H), 7.30 (d, J=5.9 Hz, 1H), 7.21 (d, J=5.0 Hz, 1H), 6.69 (s, 1H), 4.97 (s, 2H), 4.59 (s, 2H), 3.86 (s, 2H), 2.59 (d, J=14.8 Hz, 2H), 2.41 (s, 3H), 1.94 (s, 1H), 1.51 (s, 2H), 0.93 (d, J=7.0 Hz, 2H), 0.64 (d, J=5.5 Hz, 2H).

### Example 466

### Synthesis of (3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl dihydrogen phosphate (I-466)

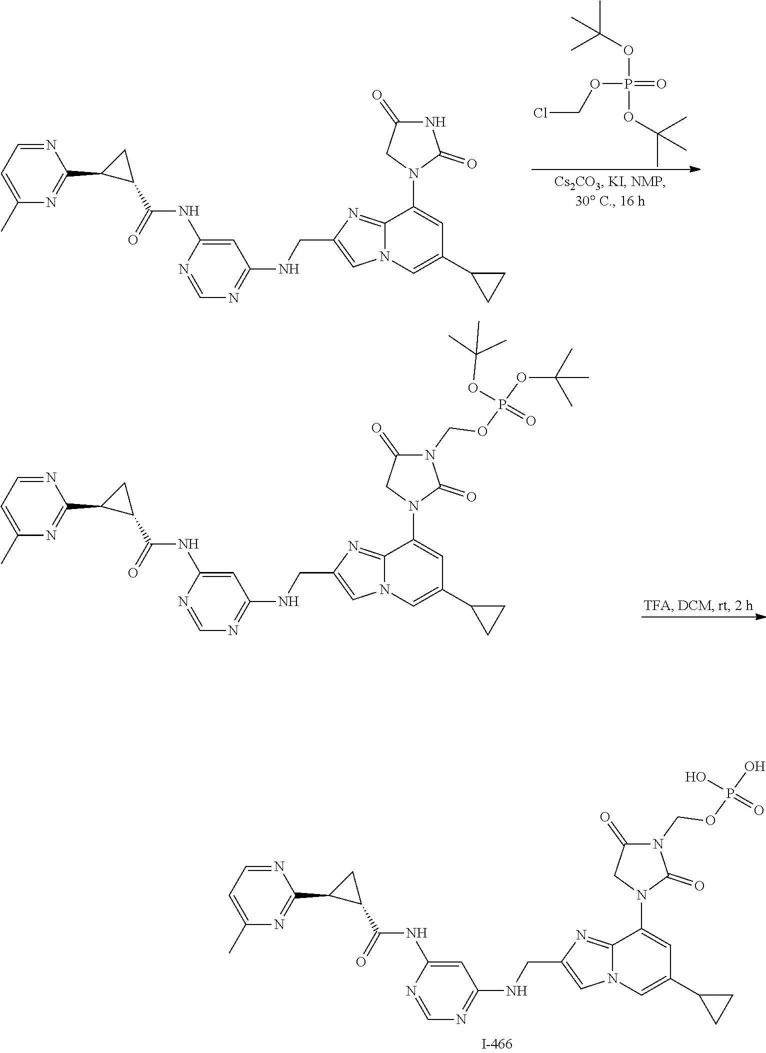

Synthesis of di-tert-butyl ((3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl) phosphate. To a mixture of (1S,2S)—N-(6-(((6-cyclopropyl-8-(2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (220 mg, 0.41 mmol), KI (136 mg, 0.82 mmol), and Cs₂CO₃ (401 mg, 1.23 mmol) in NMP (5 mL) was added di-tert-butyl (chloromethyl) phosphate (159 mg, 0.62 mmol). The reaction was stirred at room temperature for 16 h, then diluted with water (20 mL). The mixture was extracted with EtOAc (3×20 mL). The combined organic layers were washed with brine, dried over Na₂SO₄, and concentrated in vacuo to give the crude, which was purified with preparative TLC (eluent: DCM/MeOH=15:1) to give di-tert-butyl ((3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl) phosphate (50 mg, 16%) as yellow solid. ESI-MS [M+H]⁺: 761.3.

Synthesis of (3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl dihydrogen phosphate (I-466). To a solution of di-tert-butyl ((3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl) methyl) phosphate (50 mg, 0.066 mol) in DCM (10 mL) was added TFA (1 mL). After stirring at room temperature for 2 h, the reaction was neutralized with sat·aqueous NaHCO₃ (10 mL) and extracted with DCM (3×20 mL). The combined organic layers were washed with brine, dried over Na₂SO₄, and concentrated in vacuo to give the crude, which was purified with preparative TLC (eluent: DCM/MeOH=10:1) to afford (3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl dihydrogen phosphate (21.8 mg, 51%) as white solid. ESI-MS [M+H]+: 649.2 ¹H NMR (400 MHz, DMSO) δ 10.94 (s, 1H), 8.54-8.51 (m, 2H), 8.28 (s, 2H), 7.94 (s, 1H), 7.59 (s, 1H), 7.30 (s, 1H), 7.22 (d, J=5.1 Hz, 1H), 5.27 (d, J=7.3 Hz, 2H), 4.82 (s, 2H), 4.69 (s, 2H), 2.64-2.61 (m, 1H), 2.58-2.54 (m, 1H), 2.42 (s, 3H), 2.07-2.00 (m, 1H), 1.58-1.51 (m, 2H), 1.04-1.00 (m, 2H), 0.76-0.72 (m, 2H).

### Example 467

### Synthesis of 1-((((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamoyl)oxy)ethyl isobutyrate (I-467)

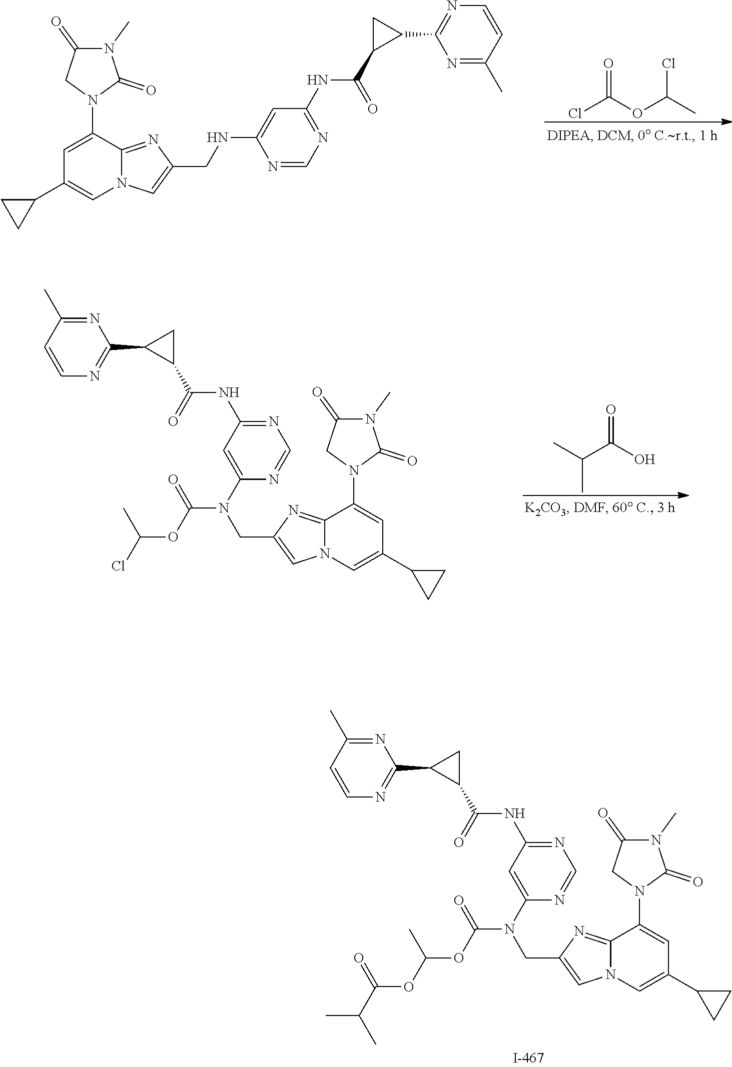

Synthesis of 1-chloroethyl ((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamate. To a solution of (1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide (200 mg, 0.36 mmol) and DIPEA (140 mg, 1.08 mmol) in DCM (20 mL) was added 1-chloroethyl carbonochloridate (102 mg, 0.72 mmol) at 0° C. After stirring at 0° C. for 1 h, the reaction was concentrated in vacuo to give the crude product of 1-chloroethyl ((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamate as a yellow solid. (236 mg, crude). ESI-MS [M+H]+: 659.2

Synthesis of 1-((((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamoyl)oxy)ethyl isobutyrate (I-467). A mixture of 1-chloroethyl ((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamate (118 mg, 0.18 mmol), DIPEA (70 mg, 0.54 mmol), and isobutyric acid (48 mg, 0.54 mmol) in DMF (6 mL) was stirred at 55° C. for 3 h. The reaction was concentrated in vacuo to give the crude product, which was purified by preparative HPLC to give product 1-((((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl)cyclopropane-1-carboxamido)pyrimidin-4-yl)carbamoyl)oxy)ethyl isobutyrate as a white solid (26 mg, 10%). ESI-MS (M+H)+: 711.3, ¹H NMR (400 MHz, DMSO) δ 11.24 (s, 1H), 8.69 (s, 1H), 8.64 (s, 1H), 8.54 (d, J=5.1 Hz, 1H), 8.19 (s, 1H), 7.61 (s, 1H), 7.34 (d, J=1.2 Hz, 1H), 7.22 (d, J=5.1 Hz, 1H), 6.87-6.75 (m, 1H), 5.30 (s, 2H), 4.81 (s, 2H), 2.96 (s, 3H), 2.73-2.65 (m, 1H), 2.61-2.52 (m, 2H), 2.42 (s, 3H), 1.98-1.84 (m, 1H), 1.64-1.51 (m, 2H), 1.47 (d, J=5.3 Hz, 3H), 1.05-0.98 (m, 3H), 0.96-0.86 (m, 5H), 0.67-0.54 (m, 2H).

### Example 468

### Synthesis of (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide (I-468)

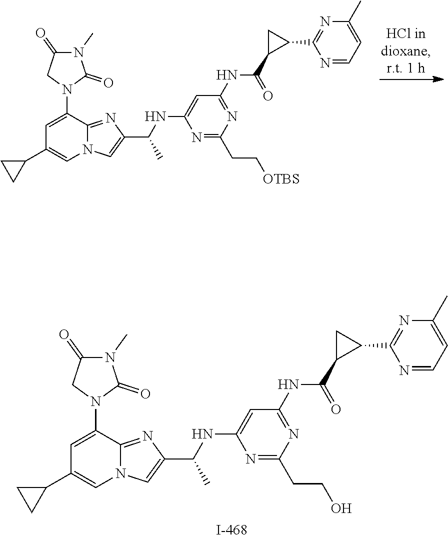 [SMILES: Cc1ccnc([C@H]2C[C@@H]2C(=O)Nc2cc(N[C@H](C)c3cn4cc(C5CC5)cc(N5CC(=O)N(C)C5=O)c4n3)nc(CCO[Si](C)(C)C(C)(C)C)n2)n1 ; Cc1ccnc([C@H]2C[C@@H]2C(=O)Nc2cc(N[C@H](C)c3cn4cc(C5CC5)cc(N5CC(=O)N(C)C5=O)c4n3)nc(CCO)n2)n1]

To a solution of (1S,2S)—N-(2-(2-((tert-butyldimethylsilyl)oxy)ethyl)-6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (50 mg, 0.07 mmol) in MeOH (3 mL) was added HCl (4M solution in dioxane, 3 mL). After stirring at room temperature for 1 h, the reaction mixture was concentrated in vacuo to give the crude product, which was purified by Pre-TLC (eluent:DCM/MeOH=15/1) to give (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide as a white solid (20 mg, 47%). ESI-MS [M+H]+: 611.3, ¹H NMR (400 MHz, DMSO) δ 10.64 (s, 1H), 8.52 (d, J=5.1 Hz, 1H), 8.23 (d, J=1.1 Hz, 1H), 7.67 (s, 2H), 7.33 (d, J=1.2 Hz, 1H), 7.20 (d, J=5.1 Hz, 1H), 7.13 (s, 1H), 5.36 (d, J=29.4 Hz, 1H), 4.94 (d, J=1.6 Hz, 2H), 4.63-4.55 (m, 1H), 3.79-3.66 (m, 2H), 2.97 (s, 3H), 2.73-2.56 (m, 4H), 2.41 (s, 3H), 1.97-1.88 (m, 1H), 1.59-1.43 (m, 5H), 1.00-0.86 (m, 2H), 0.70-0.57 (m, 2H).

The compounds in Table 35 were prepared in a similar manner to (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide from the corresponding silyl protected ethers, which were in turn prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide.

TABLE 35

| Example | Structure | Analytical Data |
|---|---|---|
| Example 469 | 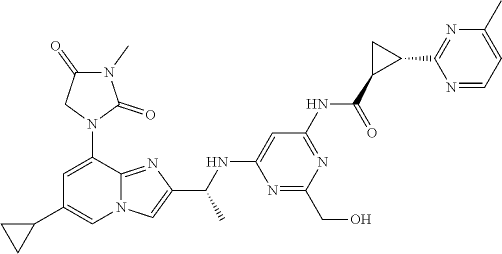 [SMILES: Cc1ccnc([C@H]2C[C@@H]2C(=O)Nc2cc(N[C@H](C)c3cn4cc(C5CC5)cc(N5CC(=O)N(C)C5=O)c4n3)nc(CO)n2)n1] | ESI-MS [M + H]⁺: 597.2, ¹H NMR (400 MHz, DMSO) δ 10.74 (s, 1H), 8.52 (d, J = 5.1 Hz, 1H), 8.23 (s, 1H), 7.80 (s, 1H), 7.67 (s, 1H), 7.34 (s, 1H), 7.23 − 7.16 (m, 2H), 4.94 (s, 2H), 4.64 (s, 1H), 4.25 (d, J = 5.8 Hz, 2H), 2.97 (s, 3H), 2.62 (s, 2H), 2.41 (s, 3H), 1.97 − 1.90 (m, 1H), 1.51 (d, J = 6.4 Hz, 5H), 0.97 − 0.89 (m, 2H), 0.64-0.58 (m, 2H). |
| | (1S,2S)-N-(6-(((R)-1-(6-cyclopropyl-8-(3- | |
| | methyl-2,4-dioxoimidazolidin-1- | |
| | yl)imidazo[1,2-a]pyridin-2- | |
| | yl)ethyl)amino)-2- | |
| | (hydroxymethyl)pyrimidin-4-yl)-2-(4- | |
| | methylpyrimidin-2-yl)cyclopropane-1- | |
| | carboxamide (I-469) | |
| Example 470 | 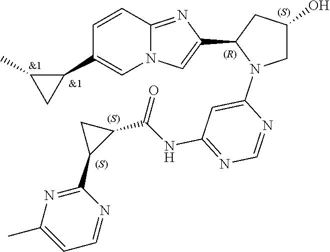 | ESI-MS [M + H]+: 511.3, ¹H NMR (400 MHz, DMSO) δ 10.77 − 10.63 (m, 1H), 8.51 (d, J = 4.9 Hz, 1H), 8.31 − 8.09 (m, 2H), 7.59 − 7.47 (m, 1H), 7.40 − 7.34 (m, 1H), 7.20 − 7.08 (m, 2H), 6.93 (d, J = 9.1 Hz, 1H), 5.28 − 4.98 (m, 2H), 4.57 − 4.46 (m, 1H), 3.82 − 3.66 (m, 2H), 2.62 − 2.54 (m, 2H), 2.41 (s, 3H), 2.33 − 2.30 (m, 2H), 1.61 − 1.44 (m, 3H), 1.23 − 1.22 (m, 1H), 1.14 (d, J = 5.9 Hz, 3H), 1.05 − 0.97 (m, 1H), 0.71 − 0.66 (m, 1H). |
| | (1S,2S)-N-(6-((2R,4S)-4-hydroxy-2-(6- | |
| | ((1S*,2S*)-2- | |
| | methylcyclopropyl)imidazo[1,2-a]pyridin- | |
| | 2-yl)pyrrolidin-1-yl)pyrimidin-4-yl)-2-(4- | |
| | methylpyrimidin-2-yl)cyclopropane-1- | |
| | carboxamide (I-470) | |
| Example 471 | 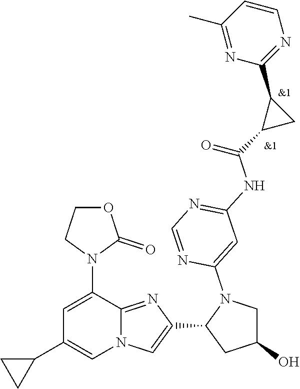 [SMILES: Cc1ccnc([C@H]2C[C@@H]2C(=O)Nc2cc(N3C[C@@H](O)C[C@@H]3c3cn4cc(C5CC5)cc(N5CCOC5=O)c4n3)ncn2)n1] | ESI-MS: [M + H]+, 582.2, ¹H NMR (400 MHz, DMSO) δ 10.79-10.64 (m, 1H), 8.51 (d, J = 4.8 Hz, 1H), 8.22 − 8.16 (m, 2H), 7.67 − 7.54 (m, 1H), 7.23 − 7.19 (m, 3H), 5.54 − 5.31 (m, 1H), 5.20 − 4.98 (m, 2H), 4.57 − 4.46 (m, 4H), 3.89 − 3.62 (m, 2H), 2.69 − 2.64 (m, 1H), 2.54 − 2.53 (m, 1H), 2.40 (s, 3H), 2.33 − 2.31 (m, 2H), 1.95 − 1.87 (m, 1H), 1.58 − 1.42 (m, 2H), 0.92 − 0.90 (m, 2H), 0.66 − 0.58 (m, 2H) |
| | rac-(1S*,2S*)-N-(6-((2R,4S)-2-(6- | |
| | cyclopropyl-8-(2-oxooxazolidin-3- | |
| | yl)imidazo[1,2-a]pyridin-2-yl)-4- | |
| | hydroxypyrrolidin-1-yl)pyrimidin-4-yl)-2- | |
| | (4-methylpyrimidin-2-yl)cyclopropane-1- | |
| | carboxamide (I-471) | |
| Example 472 | 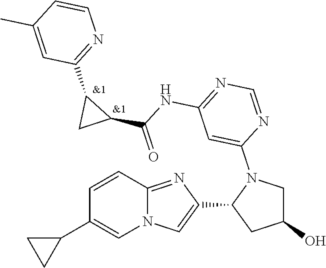 [SMILES: Cc1ccnc([C@H]2C[C@@H]2C(=O)Nc2cc(N3C[C@@H](O)C[C@@H]3c3cn4cc(C5CC5)ccc4n3)ncn2)c1] | ESI-MS [M + H]+: 496.2, ¹H NMR (400 MHz, DMSO) δ 10.70 − 10.58 (m, 1H), 8.27 − 8.13 (m, 3H), 7.64 − 7.51 (m, 1H), 7.39 − 7.37 (m, 1H), 7.28 − 7.12 (m, 2H), 7.02 − 6.95 (m, 2H), 5.19 − 4.98 (m, 2H), 4.60 − 4.46 (m, 1H), 3.84 − 3.64 (m, 2H), 2.45 − 2.17 (m, 7H), 1.95 − 1.87 (m, 1H), 1.56 − 1.39 (s, 2H), 0.93 − 0.88 (m, 2H), 0.69 − 0.62 (m, 2H). |
| | rac-(1S*,2S*)-N-(6-((2R,4S)-2-(6- | |
| | cyclopropylimidazo[1,2-a]pyridin-2-yl)-4- | |
| | hydroxypyrrolidin-1-yl)pyrimidin-4-yl)-2- | |
| | (4-methylpyridin-2-yl)cyclopropane-1- | |
| | carboxamide (I-472) | |
| Example 473 | 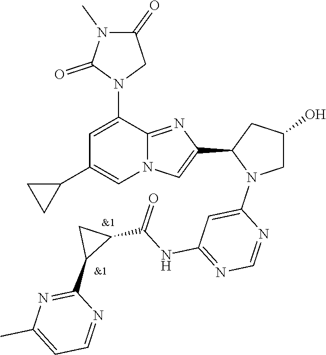 [SMILES: Cc1ccnc([C@H]2C[C@@H]2C(=O)Nc2cc(N3C[C@@H](O)C[C@@H]3c3cn4cc(C5CC5)cc(N5CC(=O)N(C)C5=O)c4n3)ncn2)n1] | ESI-MS: [M + H]+, 609.2, ¹H NMR (400 MHz, DMSO) δ 11.01 (s, 1H), 8.53 (d, J = 5.0 Hz, 1H), 8.43 − 8.19 (m, 2H), 7.95 − 7.90 (m, 1H), 7.62 (s, 1H), 7.22 − 7.16 (m, 2H), 5.51 − 5.15 (m, 2H), 4.83 − 4.66 (m, 2H), 4.53 − 4.52 (m, 1H), 3.88 − 3.84 (m, 1H), 2.99 − 2.96 (m, 3H), 2.67 − 2.56 (m, 2H), 2.41 (s, 4H), 2.33 − 2.27 (m, 1H), 2.07 − 1.99 (m, 1H), 1.60 − 1.49 (m, 2H), 1.05 − 0.96 (m, 2H), 0.78 − 0.69 (m, 2H). |
| | rac-(1S*,2S*)-N-(6-((2R,4S)-2-(6- | |
| | cyclopropyl-8-(3-methyl-2,4- | |
| | dioxoimidazolidin-1-yl)imidazo[1,2- | |
| | a]pyridin-2-yl)-4-hydroxypyrrolidin-1- | |
| | yl)pyrimidin-4-yl)-2-(4-methylpyrimidin- | |
| | 2-yl)cyclopropane-1-carboxamide (I-473) | |
| Example 474 | 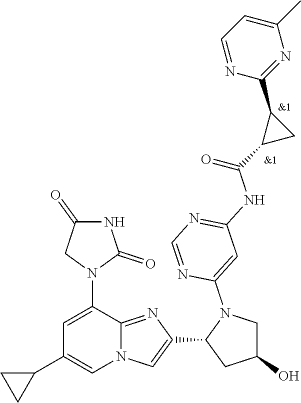 [SMILES: Cc1ccnc([C@H]2C[C@@H]2C(=O)Nc2cc(N3C[C@@H](O)C[C@@H]3c3cn4cc(C5CC5)cc(N5CC(=O)NC5=O)c4n3)ncn2)n1] | ESI-MS: [M + H]+, 595.2 ¹H NMR (400 MHz, DMSO) δ 11.30 (s, 1H), 10.75-10.60 (m, 1H), 8.49 (d, J = 5.0 Hz, 1H), 8.20 − 8.12 (m, 2H), 7.68 − 7.54 (m, 1H), 7.32 (s, 1H), 7.22 − 7.17 (m, 2H), 5.44- 5.09 (m, 2H), 4.93 − 4.85 (m, 2H), 4.56 − 4.50 (m, 1H), 3.77- 3.70 (m, 2H), 2.64 − 2.53 (m, 1H), 2.39 (s, 3H), 2.30 − 2.17 (m, 3H), 1.93 − 1.88 (m, 1H), 1.50 − 1.45 (m, 2H), 0.91 − 0.89 (m, 2H), 0.65 − 0.56 (m, 2H). |
| | rac-(1S*,2S*)-N-(6-((2R,4S)-2-(6- | |
| | cyclopropyl-8-(2,4-dioxoimidazolidin-1- | |
| | yl)imidazo[1,2-a]pyridin-2-yl)-4- | |
| | hydroxypyrrolidin-1-yl)pyrimidin-4-yl)-2- | |
| | (4-methylpyrimidin-2-yl)cyclopropane-1- | |
| | carboxamide (I-474) | |
| Example 475 | 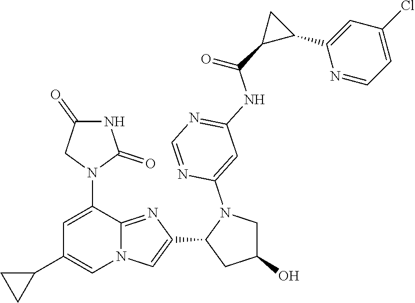 [SMILES: O=C1CN(c2cc(C3CC3)cn3cc([C@H]4C[C@H](O)CN4c4cc(NC(=O)[C@H]5C[C@@H]5c5cc(Cl)ccn5)ncn4)nc23)C(=O)N1] | ESI-MS: [M + H]+, 614.1 ¹H NMR (400 MHz, DMSO) δ 11.37 (s, 1H), 10.75 − 10.61 (m, 1H), 8.41 (d, J = 5.2 Hz, 1H), 8.22- 8.14 (m, 2H), 7.69 − 7.56 (m, 2H), 7.35 − 7.25 (m, 3H), 5.48 (s, 1H), 5.22 − 5.12 (m, 1H), 4.91 − 4.86(m, 2H), 4.63 − 4.51 (m, 1H), 3.80 − 3.72 (m, 2H), 2.67- 2.59 (m, 2H), 2.33 − 2.21 (m, 2H), 1.97 − 1.88 (m, 1H), 1.59 − 1.38 (m, 2H), 0.92 − 0.90 (m 2H), 0.63 − 0.62 (m, 2H). |
| | (1S,2S)-2-(4-chloropyridin-2-yl)-N-(6- | |
| | ((2R,4S)-2-(6-cyclopropyl-8-(2,4- | |
| | dioxoimidazolidin-1-yl)imidazo[1,2- | |
| | a]pyridin-2-yl)-4-hydroxypyrrolidin-1- | |
| | yl)pyrimidin-4-yl)cyclopropane-1- | |
| | carboxamide (I-475) | |
| Example 476 | 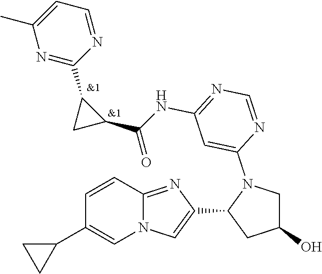 [SMILES: Cc1ccnc([C@H]2C[C@@H]2C(=O)Nc2cc(N3C[C@@H](O)C[C@@H]3c3cn4cc(C5CC5)ccc4n3)ncn2)n1] | ESI-MS [M + H]+: 497.2, ¹H NMR (400 MHz, cd3od) δ 8.45 (t, J = 5.2 Hz, 1H), 8.27 (s, 1H), 8.16 (s, 1H), 7.77 (s, 1H), 7.47 (d, J = 8.7 Hz, 1H), 7.28 (s, 2H), 7.16 (t, J = 4.6 Hz, 1H), 5.26 (s, 1H), 4.62 − 4.60 (m, 1H), 3.99 − 3.80 (m, 2H), 2.67 − 2.61 (m, 1H), 2.47 (s, 3H), 2.46 (s, 2H), 2.42 − 2.36 (m, 1H), 2.03 − 1.96 (m, 1H), 1.68 − 1.59 (m, 2H), 1.04 − 0.99 (m, 2H), 0.78 − 0.73 (m, 2H). |
| | rac-(1S*,2S*)-N-(6-((2R,4S)-2-(6- | |
| | cyclopropylimidazo[1,2-a]pyridin-2-yl)-4- | |
| | hydroxypyrrolidin-1-yl)pyrimidin-4-yl)-2- | |
| | (4-methylpyrimidin-2-yl)cyclopropane-1- | |
| | carboxamide (I-476) | |
| Example 477 | 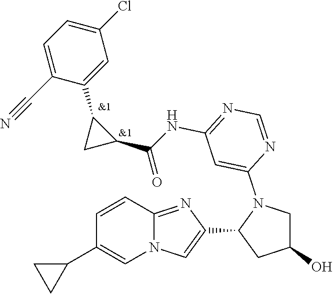 [SMILES: N#Cc1ccc(Cl)cc1[C@H]1C[C@@H]1C(=O)Nc1cc(N2C[C@@H](O)C[C@@H]2c2cn3cc(C4CC4)ccc3n2)ncn1] | ESI-MS [M + H] ⁺: 540.1, ¹H NMR (400 MHz, MeOD) δ 8.21 − 8.12 (m, 2H), 7.68 (t, J = 8.0 Hz, 2H), 7.43 − 7.40 (m, 1H), 7.35 − 7.28 (m, 3H), 7.06 − 7.02 (m, 1H), 5.54 − 5.15 (m, 1H), 4.60 (d, J = 3.5 Hz, 1H), 3.97 (s, 2H), 2.74 − 2.65 (m, 1H), 2.44 (d, J = 5.0 Hz, 2H), 2.24 (s, 1H), 1.95 − 1.88 (m, 1H), 1.68 − 1.62 (m, 1H), 1.48 (s, 1H), 0.98 − 0.90 (m, 2H), 0.70 − 0.66 (m, 2H). |
| | rac-(1S*,2S*)-2-(5-chloro-2- | |
| | cyanophenyl)-N-(6-((2R,4S)-2-(6- | |
| | cyclopropylimidazo[1,2-a]pyridin-2-yl)-4- | |
| | hydroxypyrrolidin-1-yl)pyrimidin-4- | |
| | yl)cyclopropane-1-carboxamide (I-477) | |
| Example 478 | 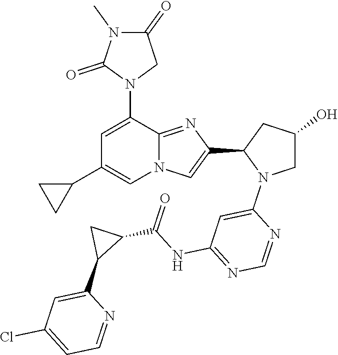 [SMILES: CN1C(=O)CN(c2cc(C3CC3)cn3cc([C@H]4C[C@H](O)CN4c4cc(NC(=O)[C@H]5C[C@@H]5c5cc(Cl)ccn5)ncn4)nc23)C1=O] | ESI-MS [M + H]+: 628.2, ¹H NMR (400 MHz, cd3od) δ 8.33 (d, J = 5.4 Hz, 1H), 8.15 (s, 1H), 8.11 (s, 1H), 7.69 (s, 1H), 7.47 (d, J = 1.8 Hz, 1H), 7.29 − 7.24 (m, 3H), 5.12 (s, 1H), 4.94 − 4.89 (m, 1H), 4.65 − 4.61 (m, 2H), 3.97 − 3.81 (m, 2H), 3.10 (s, 3H), 2.62 − 2.57 (m, 1H), 2.51 − 2.38 (m, 3H), 1.97 − 1.90 (m, 1H), 1.55 (s, 2H), 0.99 − 0.94 (m, 2H), 0.74 − 0.70 (m, 2H). |
| | (1S,2S)-2-(4-chloropyridin-2-yl)-N-(6- | |
| | ((2R,4S)-2-(6-cyclopropyl-8-(3-methyl- | |
| | 2,4-dioxoimidazolidin-1-yl)imidazo[1,2- | |
| | a]pyridin-2-yl)-4-hydroxypyrrolidin-1- | |
| | yl)pyrimidin-4-yl)cyclopropane-1- | |
| | carboxamide (I-478) | |
| Example 479 | 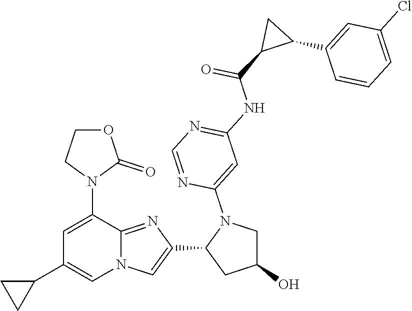 [SMILES: O=C(Nc1cc(N2C[C@@H](O)C[C@@H]2c2cn3cc(C4CC4)cc(N4CCOC4=O)c3n2)ncn1)[C@H]1C[C@@H]1c1cccc(Cl)c1] | ESI-MS [M + H]+: 600.2, ¹H NMR (400 MHz, cd3od) δ 8.36 − 8.11 (m, 2H), 7.68 (s, 1H), 7.27 − 7.07 (m, 6H), 5.16 (s, 1H), 4.62 − 4.56 (m, 3H), 4.39 (s, 1H), 4.28-4.18 (m, 1H), 4.06 − 3.80 (m, 2H), 2.46 (s, 3H), 2.24 − 2.03 (m, 1H), 1.97 − 1.91 (m, 1H), 1.51 (s, 1H), 1.32 (s, 1H), 0.99 − 0.94 (m, 2H), 0.74 − 0.70 (m, 2H). |
| | (1S,2S)-2-(3-chlorophenyl)-N-(6- | |
| | ((2R,4S)-2-(6-cyclopropyl-8-(2- | |
| | oxooxazolidin-3-yl)imidazo[1,2-a]pyridin- | |
| | 2-yl)-4-hydroxypyrrolidin-1-yl)pyrimidin- | |
| | 4-yl)cyclopropane-1-carboxamide (I-479) | |
| Example 480 | 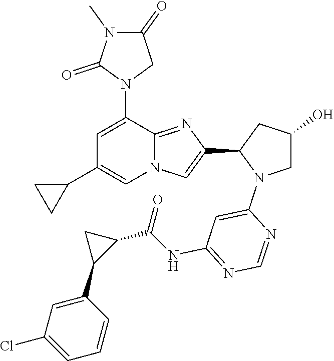 [SMILES: CN1C(=O)CN(c2cc(C3CC3)cn3cc([C@H]4C[C@H](O)CN4c4cc(NC(=O)[C@H]5C[C@@H]5c5cccc(Cl)c5)ncn4)nc23)C1=O] | ESI-MS [M + H] ⁺: 627.1, ¹H NMR (400 MHz, MeOD) δ 8.14 (d, J = 19.9 Hz, 2H), 7.69 (s, 1H), 7.29 − 7.23 (m, 3H), 7.18 (d, J = 9.5 Hz, 2H), 7.09 (d, J = 7.5 Hz, 1H), 5.12 (s, 1H), 4.95 (s, 1H), 4.66 − 4.59 (m, 2H), 3.95 (s, 2H), 3.10 (s, 3H), 2.45 (d, J = 5.5 Hz, 3H), 2.12 (s, 1H), 1.97 − 1.91 (m, 1H), 1.51 (s, 1H), 1.38 − 1.31 (m, 1H), 1.00 − 0.95 (m, 2H), 0.74 − 0.72 (m, 2H). |
| | (1S,2S)-2-(3-chlorophenyl)-N-(6- | |
| | ((2R,4S)-2-(6-cyclopropyl-8-(3-methyl- | |
| | 2,4-dioxoimidazolidin-1-yl)imidazo[1,2- | |
| | a]pyridin-2-yl)-4-hydroxypyrrolidin-1- | |
| | yl)pyrimidin-4-yl)cyclopropane-1- | |
| | carboxamide (I-480) | |
| Example 481 | 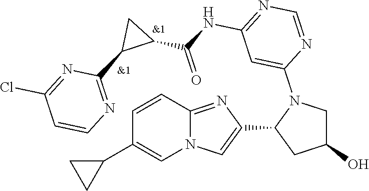 | ESI-MS [M + H]+: 517.2, ¹H NMR (400 MHz, MeOD) δ 8.55-8.47 (m, 1H), 8.14 (s, 2H), 7.65 (s, 1H), 7.40- 7.30 (m, 2H), 7.25 (s, 1H), 7.05 (d, J=8.1, 1H), 5.13 (s, 1H), 4.88 (s, 1H), 4.63-4.57 (m, 1H), 4.05-3.85 (m, 1H), 2.50 − 2.38 (m, 3H), 2.21 − 1.99 (m, 1H), 1.95-1.90 (m, 1H), 1.69 − 1.53 (m, 2H), 0.99 − 0.92 (m, 2H), 0.75 − 0.66 (m, 2H). |
| | rac-(1S*,2S*)-2-(4-chloropyrimidin-2-yl)- | |
| | N-(6-((2R,4S)-2-(6- | |
| | cyclopropylimidazo[1,2-a]pyridin-2-yl)-4- | |
| | hydroxypyrrolidin-1-yl)pyrimidin-4- | |
| | yl)cyclopropane-1-carboxamide (I-481) | |

### Example 482

### Synthesis of rac-(1S*,2S*)—N-(6-((2R,4S)-2-(6-cyclopropyl-8-(2-oxo-3-azabicyclo[3.1.0]hexan-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (I-482)

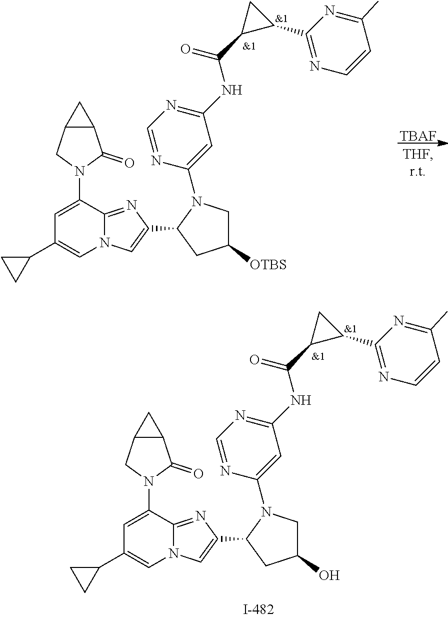

A mixture of rac-(1S*,2S*)—N-(6-((2R,4S)-4-((tert-butyldimethylsilyl)oxy)-2-(6-cyclopropyl-8-(2-oxo-3-azabicyclo[3.1.0]hexan-3-yl)imidazo[1,2-a]pyridin-2-yl)pyrrolidin-1-yl)pyrimidin-4-yl)-2-(4-methylpyrimidin-2-yl)cyclopropane-1-carboxamide (prepared in a similar manner to rac-(1S*,2S*)—N-(6-((2R,4S)-4-((tert-butyldimethylsilyl)oxy)-2-(6-cyclopropyl-8-(2-oxo-3-azabicyclo[3.1.0]hexan-3-yl)imidazo[1,2-a]pyridin-2-yl)pyrrolidin-1-yl)pyrimidin-4-yl)-2-(4-methylpyrimidin-2-yl)cyclopropane-1-carboxamide) (100 mg, 0.14 mmol) and TBAF (74 mg, 0.28 mmol) in THF (5 mL) was stirred at room temperature for 2 h. The reaction was then quenched with water (20 mL) and extracted with DCM (3×25 mL). The combined organic layer was concentrated in vacuo to give the crude product, which was purified by preparative HPLC to afford rac-(1S*,2S*)—N-(6-((2R,4S)-2-(6-cyclopropyl-8-(2-oxo-3-azabicyclo[3.1.0]hexan-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide (10 mg, 12%) as a white solid. ESI-MS [M+H]+: 592.3, ¹H NMR (400 MHz, MeOD) δ 8.46 (d, J=4.9 Hz, 1H), 8.17 (s, 1H), 8.09 (s, 1H), 7.65 (s, 1H), 7.25 (s, 1H), 7.16 (d, J=4.5 Hz, 1H), 7.01 (d, J=12.8 Hz, 1H), 5.18 (s, 1H), 4.64 (s, 1H), 4.36 (s, 1H), 4.13 (d, J=40.8 Hz, 1H), 3.96 (s, 2H), 2.67 (s, 1H), 2.46 (d, J=3.7 Hz, 5H), 2.08 (s, 1H), 2.06 (s, 1H), 1.92 (d, J=5.2 Hz, 1H), 1.63 (s, 2H), 1.29 (s, 2H), 1.13 (d, J=33.5 Hz, 1H), 0.95 (d, J=6.8 Hz, 2H), 0.71 (s, 2H).

The compounds in Table 36 were prepared in a similar manner to (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide from the corresponding silyl protected ethers (or acetylene for (1S,2S)—N-(6-((2R,4S)-2-(6-cyclopropyl imidazo[1,2-a]pyridin-2-yl)-4-hydroxypyrrolidin-1-yl)pyrimidin-4-yl)-2-(3-ethynylphenyl)cyclo propane-1-carboxamide), which were in turn prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide.

TABLE 36

| Example | Structure | Analytical Data |
|---|---|---|
| Example 483 | 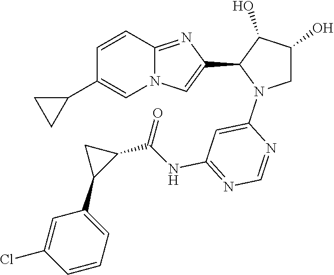 [SMILES: O=C(Nc1cc(N2C[C@@H](O)[C@@H](O)[C@@H]2c2cn3cc(C4CC4)ccc3n2)ncn1)[C@H]1C[C@@H]1c1cccc(Cl)c1] | ESI-MS [M + H]⁺: 531.2, ¹H NMR (400 MHz, CDCl₃) δ 9.73 (s, 1H), 8.21-8.13 (m, 2H), 7.79 (s, 1H), 7.44 (d, J = 9.2 Hz, 1H), 7.33 (s, 1H),7.23 − 7.14 (m, 2H), 7.11 (s, 1H), 7.03 − 6.93 (m, 2H), 5.55 (s, 1H), 5.05 (s, 1H), 4.64 (s, 2H), 4.00 (s, 2H), 3.54 (s, 1H), 2.55 (s, 1H), 1.96-1.80 (m, 1H), 1.67 (s, 1H), 1.34 (s, 1H), 1.02 − 0.87 (m, 2H), 0.69-0.59 (m, 2H). |
| | (1S,2S)-2-(3-chlorophenyl)-N-(6- | |
| | ((2S,3S,4R)-2-(6- | |
| | cyclopropylimidazo[1,2-a]pyridin-2-yl)- | |
| | 3,4-dihydroxypyrrolidin-1-yl)pyrimidin- | |
| | 4-yl)cyclopropane-1-carboxamide (I-483) | |
| Example 484 | 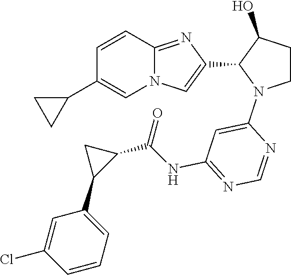 [SMILES: O=C(Nc1cc(N2CC[C@H](O)[C@H]2c2cn3cc(C4CC4)ccc3n2)ncn1)[C@H]1C[C@@H]1c1cccc(Cl)c1] | ESI-MS [M + H]+: 515.2, ¹H NMR (400 MHz, DMSO) δ 10.64 (d, J = 44.0 Hz, 1H), 8.27 − 8.10 (m, 2H), 7.52 − 7.18 (m, 6H), 7.08 − 6.97 (m, 2H), 5.37 − 5.25 (m, 2H), 4.35 (s, 1H), 3.84 − 3.71 (m, 1H), 3.59 − 3.46 (m, 1H), 2.38 − 2.28 (m, 2H), 2.17 − 2.05 (m, 1H), 1.89 − 1.79 (m, 2H), 1.51 − 1.34 (m, 2H), 0.89 − 0.87 (m, 2H), 0.66 − 0.58 (m, 2H). |
| | (1S,2S)-2-(3-chlorophenyl)-N-(6- | |
| | ((2R,3S)-2-(6-cyclopropylimidazo[1,2- | |
| | a]pyridin-2-yl)-3-hydroxypyrrolidin-1- | |
| | yl)pyrimidin-4-yl)cyclopropane-1- | |
| | carboxamide (I-484) | |
| Example 485 | 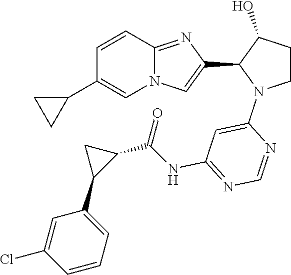 [SMILES: O=C(Nc1cc(N2CC[C@@H](O)[C@@H]2c2cn3cc(C4CC4)ccc3n2)ncn1)[C@H]1C[C@@H]1c1cccc(Cl)c1] | ESI-MS [M + H] ⁺: 515.2, ¹H NMR (400 MHz, DMSO) δ 10.66 (d, J = 45.6 Hz, 1H), 8.30 − 8.11 (m, 2H), 7.54 − 7.23 (m, 5H), 7.17 − 6.94(m, 3H), 5.39 − 5.27(m, 2H), 4.37 (d, J = 3.3 Hz, 1H), 3.86 − 3.72(m, 1H), 3.58 − 3.51 (m 1H), 2.44 − 2.30 (m, 2H), 2.13 − 2.08 (m, 1H), 1.94 − 1.83 (m, 2H), 1.52 − 1.33 (m, 2H), 0.90 − 0.85 (m, 2H), 0.64 (s, 2H). |
| | (1S,2S)-2-(3-chlorophenyl)-N-(6- | |
| | ((2S,3R)-2-(6-cyclopropylimidazo[1,2- | |
| | a]pyridin-2-yl)-3-hydroxypyrrolidin-1- | |
| | yl)pyrimidin-4-yl)cyclopropane-1- | |
| | carboxamide (I-485) | |
| Example 486 | 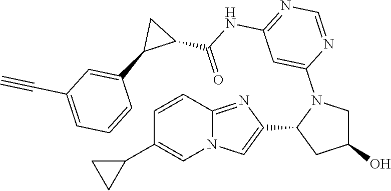 [SMILES: C#Cc1cccc([C@H]2C[C@@H]2C(=O)Nc2cc(N3C[C@@H](O)C[C@@H]3c3cn4cc(C5CC5)ccc4n3)ncn2)c1] | ESI-MS [M + H]+: 505.2, ¹H NMR (400 MHz, MeOD) δ 8.21-8.07 (m, 2H), 7.65 (s, 1H), 7.35 (d, J=9.2, 1H), 7.31 − 7.19 (m, 4H), 7.17 (s, 1H), 7.05 (d, J=7.7, 1H), 5.19 (s, 1H), 4.89 (s, 1H), 4.61-4.58 (m, 1H), 4.03 − 3.84(m, 1H), 3.46 (s, 1H), 2.51-2.38 (m, 3H), 2.12 (s, 1H), 1.95-1.89 (m, 1H), 1.50 (s, 1H), 1.34-1.28 (m, 1H), 0.99-0.91 (m, 2H), 0.74-0.66 (m, 2H). |
| | (1S,2S)-N-(6-((2R,4S)-2-(6- | |
| | cyclopropylimidazo[1,2-a]pyridin-2-yl)- | |
| | 4-hydroxypyrrolidin-1-yl)pyrimidin-4-yl)- | |
| | 2-(3-ethynylphenyl)cyclopropane-1- | |
| | carboxamide (I-486) | |

### Synthesis of (1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)-2-hydroxypyrimidin-4-yl)cyclopropane-1-carboxamide (I-487)

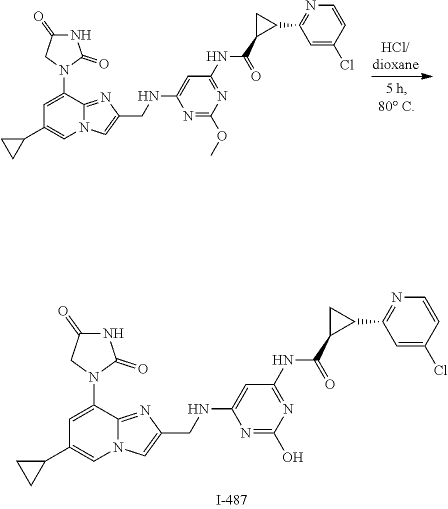

To a solution of (1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)-2-methoxypyrimidin-4-yl)cyclopropane-1-carboxamide (prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(fluoromethyl)pyrimidin-4-yl)-2-(4-methylpyrimidin-2-yl)cyclopropane-1-carboxamide) (27 mg, 0.046 mmol) in dioxane (2 mL) was added HCl/dioxane (1 mL, 4 M, 8.0 mmol) at room temperature. The mixture was stirred at 80° C. for 5 h. After cooling to room temperature, the mixture was treated with a solution of ammonia in methanol (5 mL). The resulting solution was concentrated in vacuo to give the crude product, which was purified by preparative TLC (eluent: DCM/MeOH=14/1) to give (1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)-2-hydroxypyrimidin-4-yl)cyclopropane-1-carboxamide (14 mg, 53%) as a white solid. ESI-MS [M+H]+: 574.1, ¹H NMR (400 MHz, DMSO) δ 12.28 (s, 1H), 8.38 (d, J=5.0 Hz, 1H), 8.13 (s, 1H), 7.78-7.60 (m, 3H), 7.34 (d, J=1.1 Hz, 1H), 7.29 (dd, J=5.3 Hz, 1.7 Hz, 1H), 7.24-7.11 (m, 1H), 6.26 (s, 1H), 4.72 (s, 2H), 4.45 (s, 2H), 2.57-2.54 (m, 1H), 2.43-2.42 (m, 1H), 1.91-1.84 (m, 1H), 1.50-1.40 (m, 2H), 0.91-0.86 (m, 2H), 0.62-0.58 (m, 2H).

### Example 488

### Synthesis of (1S,2S)-2-(3-chlorophenyl)-N-(4-(((6-cyclopropyl-8-(4-hydroxy-1-methylpiperidin-4-yl)imidazo[1,2-a]pyridin-2-yl)methyl)amino)pyridin-2-yl)cyclopropane-1-carboxamide (I-488)

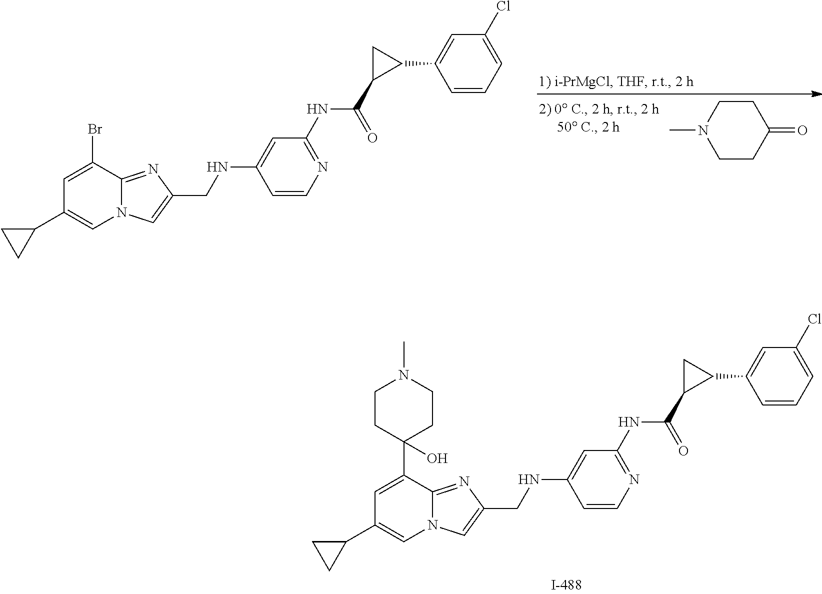

Isopropylmagnesium chloride (0.33 mL, 0.66 mmol) was added to a solution of (1S,2S)—N-(4-(((8-bromo-6-cyclopropylimidazo[1,2-a]pyridin-2-yl)methyl)amino)pyridin-2-yl)-2-(3-chlorophenyl)cyclopropane-1-carboxamide (45 mg, 0.084 mmol) in THF (1.5 mL) at 0° C. and then stirred at room temperature for 2 h under N₂. Then a solution of 1-methylpiperidin-4-one (95 mg, 0.84 mmol) in THF (0.5 mL) was added and the mixture was stirred at room temperature for 2 h and then stirred at 50° C. for additional 2 h. Water (10 mL) was added and the mixture was extracted with EtOAc (3×20 mL), the combined organic layers were concentrated in vacuo and purified by preparative TLC (DCM:MeOH=10:1) to afford (1S,2S)-2-(3-chlorophenyl)-N-(4-(((6-cyclopropyl-8-(4-hydroxy-1-methylpiperidin-4-yl)imidazo[1,2-a]pyridin-2-yl)methyl)amino)pyridin-2-yl)cyclopropane-1-carboxamide (5 mg, 10%) as a white solid. ESI-MS [M+H]+: 571.3, ¹H NMR (400 MHz, MeOD) ? 8.03 (s, 1H), 7.75 (d, J=5.9 Hz, 1H), 7.59 (s, 1H), 7.43 (s, 1H), 7.26 (t, J=8.0 Hz, 1H), 7.20-7.18 (m, 2H), 7.10 (d, J=7.6 Hz, 1H), 7.02 (d, J=1.4 Hz, 1H), 6.41 (dd, J=6.0, 2.2 Hz, 1H), 4.47 (s, 2H), 2.78 (d, J=10.6 Hz, 2H), 2.65 (t, J=11.2 Hz, 2H), 2.56-2.51 (m, 2H), 2.35 (s, 3H), 2.21-2.17 (m, 2H), 2.03-2.00 (m, 1H), 1.96-1.92 (m, 2H), 1.63-1.58 (m, 2H), 0.97-0.93 (m, 2H), 0.71-0.63 (m, 2H).

### Synthesis of rac-(1S*,2S*)—N-(6-chloropyrimidin-4-yl)-2-(5-fluoro-4-methylpyrimidin-2-yl) cyclopropane-1-carboxamide

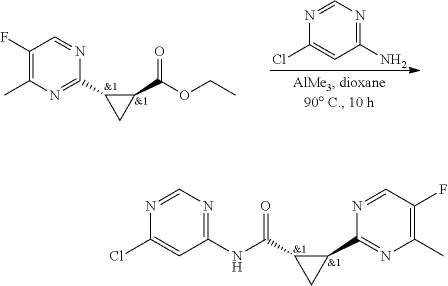 [SMILES: CCOC(=O)[C@H]1C[C@@H]1c1ncc(F)c(C)n1 ; Nc1cc(Cl)ncn1 ; Cc1nc([C@H]2C[C@@H]2C(=O)Nc2cc(Cl)ncn2)ncc1F]

To a solution of 6-chloropyrimidin-4-amine (344 mg, 2.67 mmol) in dioxane (7 mL) was added AlMe₃ (1.6 M solution in toluene, 2.2 mL, 3.56 mmol) at 25° C. After stirring at 30° C. for 2 h under N₂, a solution of rac-ethyl (1S*,2S*)-2-(5-fluoro-4-methylpyrimidin-2-yl)cyclopropane-1-carboxylate (200 mg, 0.89 mmol) in dioxane (3 mL) was added dropwise. The resulting reaction mixture was stirred at 90° C. for another 10 h. After cooling to room temperature, the reaction was quenched with water (30 mL) and extracted with EtOAc (3×40 mL). The combined organic layers were washed with brine, dried over anhydrous Na₂SO₄, and concentrated in vacuo to give the crude, which was purified by silica gel chromatography (eluent: DCM/MeOH=20/1) to give rac-(1S*,2S*)—N-(6-chloropyrimidin-4-yl)-2-(5-fluoro-4-methylpyrimidin-2-yl)cyclopropane-1-carboxamide (60 mg, 22%) as a yellow solid. ESI-MS [M+H]+: 308.1, ¹H NMR (400 MHz, DMSO) δ 11.61 (s, 1H), 8.75 (d, J=0.7 Hz, 1H), 8.63 (d, J=1.7 Hz, 1H), 8.13 (d, J=0.7 Hz, 1H), 2.74-2.63 (m, 2H), 2.44 (s, 3H), 1.63-1.53 (m, 2H).

The compounds in Table 37 were prepared in a similar fashion to rac-(1S*,2S*)—N-(6-chloropyrimidin-4-yl)-2-(5-fluoro-4-methylpyrimidin-2-yl)cyclopropane-1-carboxamide from a substituted cyclopropyl ester and an appropriate heterocyclic amine.

TABLE 37

| | Analytical |
|---|---|
| Compound | Data |
| 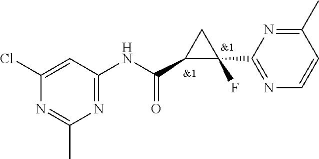 [SMILES: Cc1ccnc([C@@]2(F)C[C@@H]2C(=O)Nc2cc(Cl)nc(C)n2)n1] | ESI-MS [M + H]+: 322.3 |
| rac-(1S*,2S*)-N-(6-chloro-2-methylpyrimidin-4-yl)- | |
| 2-fluoro-2-(4-methylpyrimidin-2-yl)cyclopropane-1- | |
| carboxamide | |
| 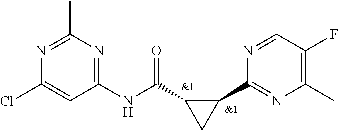 [SMILES: Cc1nc(Cl)cc(NC(=O)[C@H]2C[C@@H]2c2ncc(F)c(C)n2)n1] | ESI-MS [M + H]⁺: 322.3 |
| rac-(1S*,2S*)-N-(6-chloro-2-methylpyrimidin-4-yl)- | |
| 2-(5-fluoro-4-methylpyrimidin-2-yl)cyclopropane-1- | |
| carboxamide | |
| 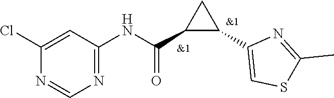 [SMILES: Cc1nc([C@H]2C[C@@H]2C(=O)Nc2cc(Cl)ncn2)cs1] | ESI-MS [M + H]+: 295.2 |
| rac-(1S*,2S*)-N-(6-chloropyrimidin-4-yl)-2-(2- | |
| methylthiazol-4-yl)cyclopropane-1-carboxamide | |
| 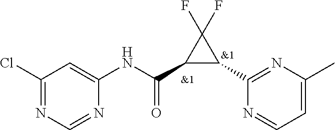 [SMILES: Cc1ccnc([C@@H]2[C@@H](C(=O)Nc3cc(Cl)ncn3)C2(F)F)n1] | ESI-MS [M + H] +: 326.2 |
| rac-(1S*,2S*)-N-(6-chloropyrimidin-4-yl)-2,2- | |
| difluoro-3-(4-methylpyrimidin-2-yl)cyclopropane-1- | |
| carboxamide | |
| 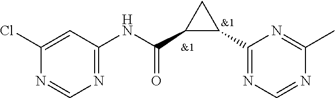 [SMILES: Cc1ncnc([C@H]2C[C@@H]2C(=O)Nc2cc(Cl)ncn2)n1] | ESI-MS [M + H] +: 291.2 |
| rac-(1S*,2S*)-N-(6-chloropyrimidin-4-yl)-2-(4- | |
| methyl-1,3,5-triazin-2-yl)cyclopropane-1-carboxamide | |
| 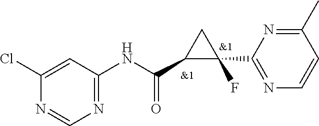 [SMILES: Cc1ccnc([C@@]2(F)C[C@@H]2C(=O)Nc2cc(Cl)ncn2)n1] | ESI-MS [M + H]⁺: 308.2 |
| rac-(1S*,2S*)-N-(6-chloropyrimidin-4-yl)-2-fluoro-2- | |
| (4-methylpyrimidin-2-yl)cyclopropane-1-carboxamide | |
| 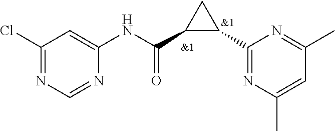 [SMILES: Cc1cc(C)nc([C@H]2C[C@@H]2C(=O)Nc2cc(Cl)ncn2)n1] | ESI-MS [M + H]+: 304.2. |
| rac-(1S*,2S*)-N-(6-chloropyrimidin-4-yl)-2-(4,6- | |
| dimethylpyrimidin-2-yl)cyclopropane-1-carboxamide | |
| 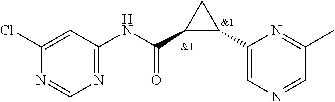 [SMILES: Cc1cncc([C@H]2C[C@@H]2C(=O)Nc2cc(Cl)ncn2)n1] | ESI-MS [M + H]+: 290.2. |
| rac-(1S*,2S*)-N-(6-chloropyrimidin-4-yl)-2-(6- | |
| methylpyrazin-2-yl)cyclopropane-1-carboxamide | |
| 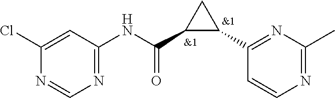 [SMILES: Cc1nccc([C@H]2C[C@@H]2C(=O)Nc2cc(Cl)ncn2)n1] | ESI-MS [M + H] +: 290.2 |
| rac-(1S*,2S*)-N-(6-chloropyrimidin-4-yl)-2-(2- | |
| methylpyrimidin-4-yl)cyclopropane-1-carboxamide | |
| 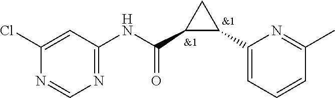 [SMILES: Cc1cccc([C@H]2C[C@@H]2C(=O)Nc2cc(Cl)ncn2)n1] rac-(1S*,2S*)-N-(6-chloropyrimidin-4-yl)-2-(6- methylpyridin-2-yl)cyclopropane-1-carboxamide | ESI-MS [M + H]+: 289.3. ¹H NMR (400 MHz, DMSO) δ 11.52 (s, 1H), 8.74 (s, 1H), 8.14 (s, 1H), |
| | 7.57 (t, |
| | J = 7.6 |
| | Hz, 1H), |
| | 7.22 (d, |
| | J = 7.6 Hz, |
| | 1H), 7.07 |
| | (d, J = 7.5 |
| | Hz, 1H), |
| | 2.63 − 2.57 |
| | (m, 2H), |
| | 2.41 (s, 3H), |
| | 1.60 − 1.56 |
| | (m, 1H), |
| | 1.53 − 1.48 |
| | (m, 1H). |
| 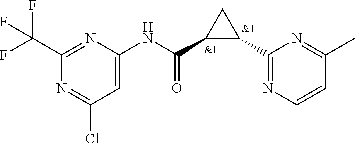 [SMILES: Cc1ccnc([C@H]2C[C@@H]2C(=O)Nc2cc(Cl)nc(C(F)(F)F)n2)n1] | ESI-MS [M + H]+: 358.3. |
| rac-(1S*,2S*)-N-(6-chloro-2- | |
| (trifluoromethyl)pyrimidin-4-yl)-2-(4- | |
| methylpyrimidin-2-yl)cyclopropane-1-carboxamide | |
| 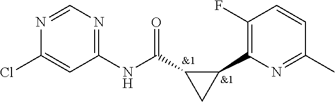 [SMILES: Cc1ccc(F)c([C@H]2C[C@@H]2C(=O)Nc2cc(Cl)ncn2)n1] | ESI-MS [M + H]+: 307.2 |
| rac-(1S*,2S*)-N-(6-chloropyrimidin-4-yl)-2-(3- | |
| fluoro-6-methylpyridin-2-yl)cyclopropane-1- | |
| carboxamide | |
| 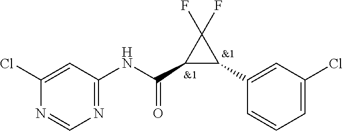 [SMILES: O=C(Nc1cc(Cl)ncn1)[C@@H]1[C@@H](c2cccc(Cl)c2)C1(F)F] | ESI-MS [M + H] +: 344.2 |
| rac-(1S*,3S*)-3-(3-chlorophenyl)-N-(6- | |
| chloropyrimidin-4-yl)-2,2-difluorocyclopropane-1- | |
| carboxamide | |
| 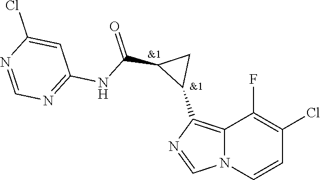 [SMILES: O=C(Nc1cc(Cl)ncn1)[C@H]1C[C@@H]1c1ncn2ccc(Cl)c(F)c12] | ESI-MS [M + H]+: 366.2 |
| rac-(1S*,2S*)-2-(7-chloro-8-fluoroimidazo[1,5- | |
| a]pyridin-1-yl)-N-(6-chloropyrimidin-4- | |
| yl)cyclopropane-1-carboxamide | |
| 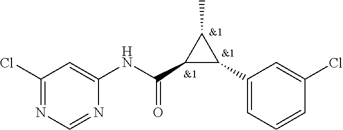 [SMILES: C[C@H]1[C@H](C(=O)Nc2cc(Cl)ncn2)[C@H]1c1cccc(Cl)c1] | ESI-MS [M + H] +: 322.2 |
| rac-(1R*,2R*,3S*)-2-(3-chlorophenyl)-N-(6- | |
| chloropyrimidin-4-yl)-3-methylcyclopropane-1- | |
| carboxamide | |
| 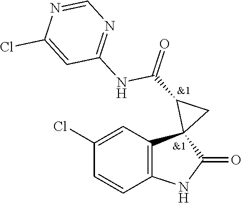 [SMILES: O=C(Nc1cc(Cl)ncn1)[C@@H]1C[C@]12C(=O)Nc1ccc(Cl)cc12] | ESI-MS [M + H] +: 349.3 |
| rac-(1R*,2S*)-5′-chloro-N-(6-chloropyrimidin-4-yl)- | |
| 2′-oxospiro[cyclopropane-1,3′-indoline]-2- | |
| carboxamide | |
| 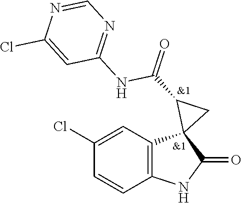 [SMILES: O=C(Nc1cc(Cl)ncn1)[C@@H]1C[C@@]12C(=O)Nc1ccc(Cl)cc12] | ESI-MS [M + H]+: 349.1 |
| rac-(1R*,2R*)-5′-chloro-N-(6-chloropyrimidin-4-yl)- | |
| 2′-oxospiro[cyclopropane-1,3′-indoline]-2- | |
| carboxamide | |
| 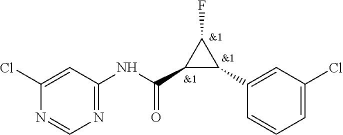 [SMILES: O=C(Nc1cc(Cl)ncn1)[C@H]1[C@H](F)[C@@H]1c1cccc(Cl)c1] | ESI-MS [M + H]+: 326.1. |
| rac-(1R*,2R*,3S*)-2-(3-chlorophenyl)-N-(6- | |
| chloropyrimidin-4-yl)-3-fluorocyclopropane-1- | |
| carboxamide | |
| 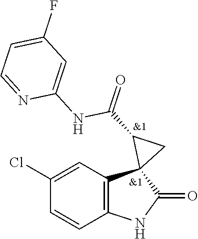 [SMILES: O=C(Nc1cc(F)ccn1)[C@@H]1C[C@]12C(=O)Nc1ccc(Cl)cc12] | ESI-MS [M + H]+: 332.1. |
| rac-(1R*,2S*)-5′-chloro-N-(4-fluoropyridin-2-yl)-2′- | |
| oxospiro[cyclopropane-1,3′-indoline]-2-carboxamide | |
| 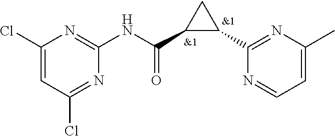 [SMILES: Cc1ccnc([C@H]2C[C@@H]2C(=O)Nc2nc(Cl)cc(Cl)n2)n1] | ESI-MS [M + H]+: 324.1. |
| rac-(1S*,2S*)-N-(4,6-dichloropyrimidin-2-yl)-2-(4- | |
| methylpyrimidin-2-yl)cyclopropane-1-carboxamide | |
| 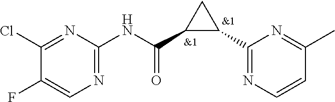 [SMILES: Cc1ccnc([C@H]2C[C@@H]2C(=O)Nc2ncc(F)c(Cl)n2)n1] | ESI-MS [M + H]+: 308.1. |
| rac-(1S*,2S*)-N-(4-chloro-5-fluoropyrimidin-2-yl)-2- | |
| (4-methylpyrimidin-2-yl)cyclopropane-1-carboxamide | |
| 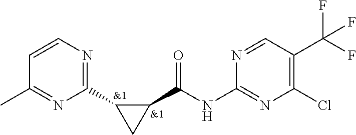 [SMILES: Cc1ccnc([C@H]2C[C@@H]2C(=O)Nc2ncc(C(F)(F)F)c(Cl)n2)n1] | ESI-MS [M + H]+: 358.1. |
| rac-(1S*,2S*)-N-(4-chloro-5- | |
| (trifluoromethyl)pyrimidin-2-yl)-2-(4- | |
| methylpyrimidin-2-yl)cyclopropane-1-carboxamide | |
| 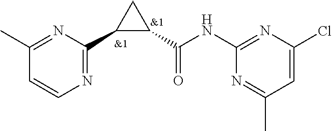 [SMILES: Cc1cc(Cl)nc(NC(=O)[C@H]2C[C@@H]2c2nccc(C)n2)n1] | ESI-MS [M + H]+: 304.1. |
| rac-(1S*,2S*)-N-(4-chloro-6-methylpyrimidin-2-yl)- | |
| 2-(4-methylpyrimidin-2-yl)cyclopropane-1- | |
| carboxamide | |
| 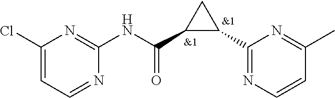 [SMILES: Cc1ccnc([C@H]2C[C@@H]2C(=O)Nc2nccc(Cl)n2)n1] | ESI-MS [M + H]+: 290.1. |
| rac-(1S*,2S*)-N-(4-chloropyrimidin-2-yl)-2-(4- | |
| methylpyrimidin-2-yl)cyclopropane-1-carboxamide | |
| 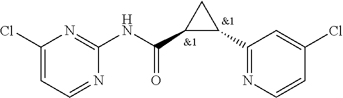 [SMILES: O=C(Nc1nccc(Cl)n1)[C@H]1C[C@@H]1c1cc(Cl)ccn1] | ESI-MS [M + H]+: 309.1. |
| rac-(1S*,2S*)-2-(4-chloropyridin-2-yl)-N-(4- | |
| chloropyrimidin-2-yl)cyclopropane-1-carboxamide | |
| 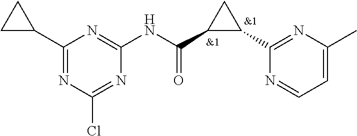 [SMILES: Cc1ccnc([C@H]2C[C@@H]2C(=O)Nc2nc(Cl)nc(C3CC3)n2)n1] | ESI-MS [M + H]+: 331.2. |
| rac-(1S*,2S*)-N-(4-chloro-6-cyclopropyl-1,3,5- | |
| triazin-2-yl)-2-(4-methylpyrimidin-2-yl)cyclopropane- | |
| 1-carboxamide | |
| 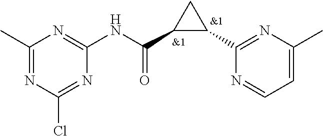 | ESI-MS [M + H]+: 305.2. |
| rac-(1S*,2S*)-N-(4-chloro-6-methyl-1,3,5-triazin-2- | |
| yl)-2-(4-methylpyrimidin-2-yl)cyclopropane-1- | |
| carboxamide | |
| 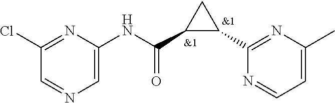 [SMILES: Cc1ccnc([C@H]2C[C@@H]2C(=O)Nc2cncc(Cl)n2)n1] | ESI-MS [M + H]+: 290.1. |
| rac-(1S*,2S*)-N-(6-chloropyrazin-2-yl)-2-(4- | |
| methylpyrimidin-2-yl)cyclopropane-1-carboxamide | |
| 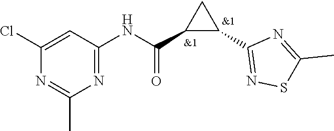 [SMILES: Cc1nc(Cl)cc(NC(=O)[C@H]2C[C@@H]2c2nsc(C)n2)n1] | ESI-MS [M + H]+: 310.2. |
| rac-(1S*,2S*)-N-(6-chloro-2-methylpyrimidin-4-yl)- | |
| 2-(5-methyl-1,2,4-thiadiazol-3-yl)cyclopropane-1- | |
| carboxamide | |
| 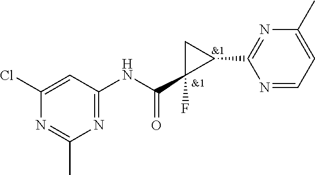 [SMILES: Cc1ccnc([C@H]2C[C@]2(F)C(=O)Nc2cc(Cl)nc(C)n2)n1] | ESI-MS [M + H]+: 322.1. |
| rac-(1S*,2S*)-N-(6-chloro-2-methylpyrimidin-4-yl)- | |
| 1-fluoro-2-(4-methylpyrimidin-2-yl)cyclopropane-1- | |
| carboxamide | |
| 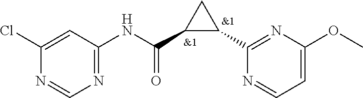 [SMILES: COc1ccnc([C@H]2C[C@@H]2C(=O)Nc2cc(Cl)ncn2)n1] | ESI-MS [M + H]+: 306.2 |
| rac-(1R,2R)-N-(6-chloropyrimidin-4-yl)-2-(4- | |
| methoxypyrimidin-2-yl)cyclopropane-1-carboxamide | |
| 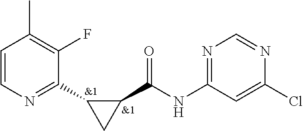 [SMILES: Cc1ccnc([C@H]2C[C@@H]2C(=O)Nc2cc(Cl)ncn2)c1F] | ESI-MS [M + H]+: 307.1. |
| rac-(1S*,2S*)-N-(6-chloropyrimidin-4-yl)-2-(3- | |
| fluoro-4-methylpyridin-2-yl)cyclopropane-1- | |
| carboxamide | |
| 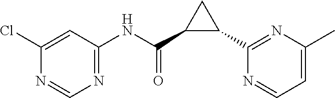 [SMILES: Cc1ccnc([C@H]2C[C@@H]2C(=O)Nc2cc(Cl)ncn2)n1] | ESI-MS [M + H]+: 290.2. |
| (1S,2S)-N-(6-chloropyrimidin-4-yl)-2-(4- | |
| methylpyrimidin-2-yl)cyclopropane-1-carboxamide | |
| 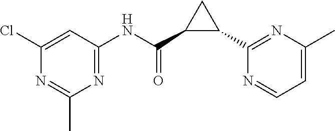 [SMILES: Cc1ccnc([C@H]2C[C@@H]2C(=O)Nc2cc(Cl)nc(C)n2)n1] | ESI-MS [M + H]+: 304.2. |
| (1S,2S)-N-(6-chloro-2-methylpyrimidin-4-yl)-2-(4- | |
| methylpyrimidin-2-yl)cyclopropane-1-carboxamide | |

### Example 489

### Synthesis of rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide (I-489)

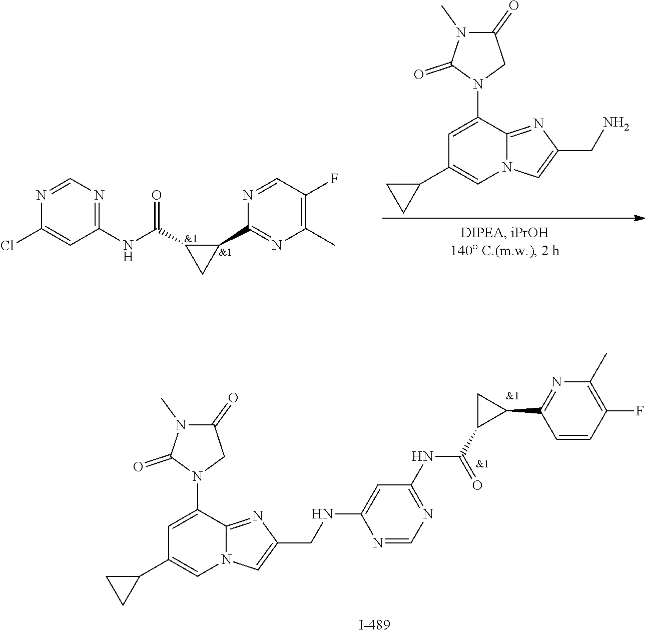 [SMILES: Cc1nc([C@H]2C[C@@H]2C(=O)Nc2cc(Cl)ncn2)ncc1F ; CN1C(=O)CN(c2cc(C3CC3)cn3cc(CN)nc23)C1=O ; Cc1nc([C@@H]2C[C@H]2C(=O)Nc2cc(NCc3cn4cc(C5CC5)cc(N5CC(=O)N(C)C5=O)c4n3)ncn2)ccc1F]

A mixture of rac-(1S*,2S*)—N-(6-chloropyrimidin-4-yl)-2-(5-fluoro-4-methylpyrimidin-2-yl)cyclopropane-1-carboxamide (30 mg, 0.098 mmol), 1-(2-(aminomethyl)-6-cyclopropylimidazo[1,2-a]pyridin-8-yl)-3-methylimidazolidine-2,4-dione (29 mg, 0.098 mmol), and DIPEA (37 mg, 0.29 mmol) in i-PrOH (2 mL) was stirred in a sealed tube. After degassing with N₂ for 1 min, the reaction mixture was irradiated in a microwave reactor at 140° C. for 2 h. The reaction was then cooled to room temperature and concentrated in vacuo to give the crude, which was purified by preparative TLC (eluent: DCM/MeOH=15/1) to give the title compound (20.1 mg, 36%) as a white solid. ESI-MS [M+H]+: 571.3, ¹H NMR (400 MHz, DMSO) δ 10.69 (s, 1H), 8.62 (s, 1H), 8.22 (d, J=22.4 Hz, 2H), 7.89 (s, 1H), 7.69 (s, 1H), 7.32 (d, J=11.4 Hz, 2H), 4.91 (s, 2H), 4.58 (s, 2H), 2.97 (s, 3H), 2.67-2.66 (m, 1H), 2.46-2.45 (m, 1H), 2.43 (s, 3H), 1.93-1.92 (m, 1H), 1.50-1.49 (m, 2H), 0.94-0.93 (m, 2H), 0.64-0.63 (m, 2H). ESI-MS [M+H]+: 621.3.

The compounds in Table 38 were prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide from a suitable amine and the indicated coupling partner.

TABLE 38

| | | Coupling Partner |
|---|---|---|
| Example | Structure | Analytical Data |
| Example 490 | 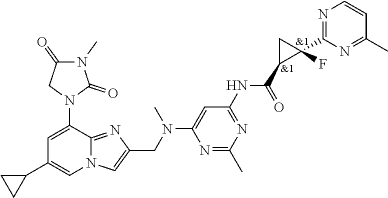 [SMILES: Cc1ccnc([C@@]2(F)C[C@@H]2C(=O)Nc2cc(N(C)Cc3cn4cc(C5CC5)cc(N5CC(=O)N(C)C5=O)c4n3)nc(C)n2)n1] | rac-(1S*,2S*)-N-(6-chloro- 2-methylpyrimidin- 4-yl)-2-fluoro-2-(4-methylpyrimidin-2- yl)cyclopropane-1-carboxamide ESI-MS (M + H)+: 599.2, ¹H NMR (400 MHz, DMSO) δ 10.77 (s, 1H), 8.66 (d, J = 5.1 Hz, 1H), 8.21 (s, 1H), 7.67 (s, 1H), 7.36-7.31 (m, 3H), 4.91 − 4.82 (m, 4H), 3.12 (s, 3H), 2.97 (s, 3H), 2.91 (s, 1H), 2.48 (s, 3H), 2.29 (s, 3H), 2.22 − 2.16 (m, 1H), 1.97 − 1.85 (m, 2H), 0.96 − 0.91 (m, 2H), 0.66 − 0.62 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)(methyl)amino)-2- | |
| | methylpyrimidin-4-yl)-2-fluoro- | |
| | 2-(4-methylpyrimidin-2- | |
| | yl)cyclopropane-1-carboxamide | |
| | (I-490) | |
| Example 491 | 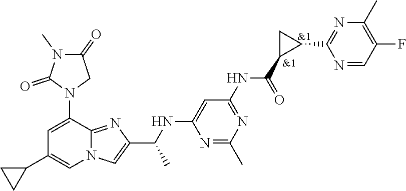 [SMILES: Cc1nc(NC(=O)[C@H]2C[C@@H]2c2ncc(F)c(C)n2)cc(N[C@H](C)c2cn3cc(C4CC4)cc(N4CC(=O)N(C)C4=O)c3n2)n1] | rac-(1S*,2S*)-N-(6-chloro-2-methylpyrimidin- 4-yl)-2-(5-fluoro-4-methylpyrimidin-2- yl)cyclopropane-1-carboxamide ESI-MS (M + H)+: 599.2, ¹H NMR (400 MHz, DMSO) δ 10.66 (s, 1H), 8.61 (d, J = 1.5 Hz, 1H), 8.24 (s, 1H), 7.68 − 7.64 (m, 2H), 7.34 (d, J = 1.6 Hz, 1H), 7.12 (s, 1H), 5.40 (s, 1H), 4.95 (s, 2H), 2.98 (s, 3H), 2.51-2.49 (m, 2H), 2.43 (d, J = 2.2 Hz, 3H), 2.25 (s, 3H), 1.97 − 1.90 (m, 1H), 1.50-1.49 (m, 5H), 0.94-0.92 (m, 2H), 0.64-0.63 (m, 2H). |
| | rac-(-(1S*,2S*)-N-(6-(((R)-1- | |
| | (6-cyclopropyl-8-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(5- | |
| | fluoro-4-methylpyrimidin-2- | |
| | yl)cyclopropane-1-carboxamide | |
| | (I-491) | |
| Example 492 | 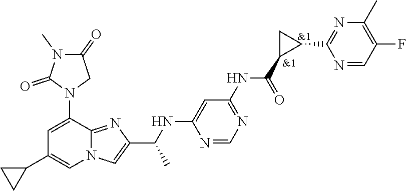 [SMILES: Cc1nc([C@H]2C[C@@H]2C(=O)Nc2cc(N[C@H](C)c3cn4cc(C5CC5)cc(N5CC(=O)N(C)C5=O)c4n3)ncn2)ncc1F] | rac-(1S*,2S*)-N-(6-chloropyrimidin-4-yl)-2-(5- fluoro-4-methylpyrimidin-2-yl)cyclopropane-1- carboxamide ESI-MS (M + H)+: 585.2, ¹H NMR (400 MHz, DMSO) δ 10.66 (s, 1H), 8.62 (d, J = 1.5 Hz, 1H), 8.25-8.16 (m, 2H), 7.78 (s, 1H), 7.65 (d, J = 2.1 Hz, 1H), 7.34-7.30 (m, 2H), 5.33 (s, 1H), 4.93 (s, 2H), 2.97 (s, 3H), 2.55-2.51 (m, 2H), 2.43 (s, 3H), 1.93-1.92 (m, 1H), 1.59 − 1.46 (m, 5H), 0.96-0.92 (m, 2H), 0.63-0.62 (m, 2H). |
| | rac-(-(1S*,2S*)-N-(6-(((R)-1- | |
| | (6-cyclopropyl-8-(3-methyl-2,4- | |
| | dioxoimidazolidin-1- | |
| | yl)imidazo[1,2-a]pyridin-2- | |
| | yl)ethyl)amino)pyrimidin-4-yl)- | |
| | 2-(5-fluoro-4-methylpyrimidin- | |
| | 2-yl)cyclopropane-1- | |
| | carboxamide (I-492) | |
| Example 493 | 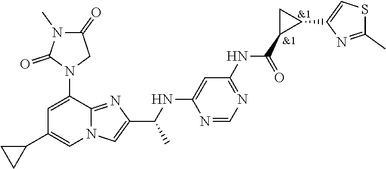 | rac-(1S*,2S*)-N-(6-chloropyrimidin-4-yl)-2- (2-methylthiazol-4-yl)cyclopropane-1- carboxamide ESI-MS (M + H)+: 558.2, ¹H NMR (400 MHz, DMSO) δ 10.60 (s, 1H), 8.24 (s, 1H), 8.18 (s, 1H), 7.87 (s, 1H), 7.68 (s, 1H), 7.33 (s, 1H), 7.30 (s, 1H), 7.25 (s, 1H), 4.91 (s, 2H), 4.57 (s, 2H), 2.97 (s, 3H), 2.60 (s, 3H), 2.54 − 2.52 (m, 1H), 2.46 − 2.40 (m, 1H), 1.98 − 1.89 (m, 1H), 1.45 − 1.37 (m, 2H), 0.97 − 0.90 (m, 2H), 0.68 − 0.58 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2-(2-methylthiazol-4- | |
| | yl)cyclopropane-1-carboxamide | |
| | (I-493) | |
| Example 494 | 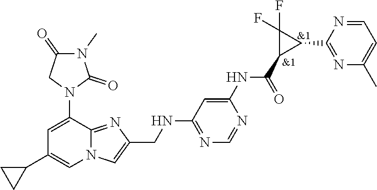 [SMILES: Cc1ccnc([C@@H]2[C@@H](C(=O)Nc3cc(NCc4cn5cc(C6CC6)cc(N6CC(=O)N(C)C6=O)c5n4)ncn3)C2(F)F)n1] | rac-(1S*,3S*)-N-(6-chloropyrimidin-4-yl)-2,2- difluoro-3-(4-methylpyrimidin-2- yl)cyclopropane-1-carboxamide ESI-MS (M + H)+: 589.2, ¹H NMR (400 MHz, DMSO) δ 11.02 (s, 1H), 8.66 (d, J = 5.1 Hz, 1H), 8.24 (s, 2H), 7.97 (s, 1H), 7.70 (s, 1H), 7.37 (d, J = 5.1 Hz, 1H), 7.34 − 7.29 (m, 2H), 4.91 (s, 2H), 4.60 (s, 2H), 3.97 − 3.91 (m, 1H), 3.66 − 3.60 (m, 1H), 2.48 (s, 3H) 2.97 (s, 3H), 1.95 − 1.91 (m, 1H), 0.96 − 0.91 (m, 2H), 0.65 − 0.62 (m, 2H). |
| | rac-(1S*,3S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2,2-difluoro-3-(4- | |
| | methylpyrimidin-2- | |
| | yl)cyclopropane-1-carboxamide | |
| | (I-494) | |
| Example 495 | 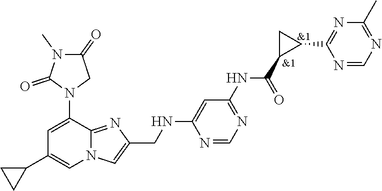 [SMILES: Cc1ncnc([C@H]2C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2-(4- methyl-1,3,5-triazin-2-yl)cyclopropane-1- carboxamide ESI-MS (M + H)+: 554.2, ¹H NMR (400 MHz, DMSO) δ 10.74 (s, 1H), 9.00 (s, 1H), 8.24 (d, J = 1.0 Hz, 1H), 8.19 (s, 1H), 7.91 (s, 1H), 7.69 (s, 1H), 7.33 (d, J = 1.0 Hz, 1H), 7.30 (d, J = 0.6 Hz, 1H), 4.91 (s, 2H), 4.58 (s, 2H), 2.97 (s, 3H), 2.80 − 2.72 (m, 1H), 2.54 (s, 3H), 2.48 − 2.42 (m, 1H), 1.99-1.89 (m, 1H), 1.65 − 1.56 (m, 2H), 0.98-0.89 (m, 2H), 0.68 − 0.60 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2-(4-methyl-1,3,5-triazin-2- | |
| | yl)cyclopropane-1-carboxamide | |
| | (1-495) | |
| Example 496 | 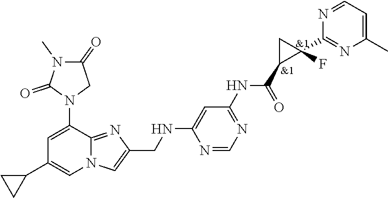 [SMILES: Cc1ccnc([C@@]2(F)C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2- fluoro-2-(4-methylpyrimidin-2- yl)cyclopropane-1-carboxamide ESI-MS (M + H)+: 571.2, 1H NMR (400 MHz, DMSO) δ 10.70 (s, 1H), 8.67 (d, J = 5.1 Hz, 1H), 8.28 − 8.16 (m, 2H), 7.93 (s, 1H), 7.69 (s, 1H), 7.36 − 7.33 (m, 3H), 4.91 (s, 2H), 4.59 (s, 2H), 2.97 (s, 3H), 2.92 (t, J = 7.3 Hz, 1H), 2.51 (s, 3H), 2.26 − 2.17 (m, 1H), 1.97 − 1.87 (m, 2H), 0.96 − 0.91 (m, 2H), 0.65 − 0.63 (m, 2H). |
| | rac-(1S*,2S*)-N-(6-(((6- | |
| | cyclopropyl-8-(3-methyl-2,4- | |
| | dioxoimidazolidin-1-yl)imidazo | |
| | [1,2-a]pyridin-2-yl)methyl) | |
| | amino)pyrimidin-4-yl)-2-fluoro- | |
| | 2-(4-methylpyrimidin-2-yl)cyclo | |
| | propane-1-carboxamide (I-496) | |
| Example 497 | 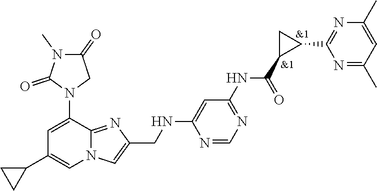 [SMILES: Cc1cc(C)nc([C@H]2C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2- (4,6-dimethylpyrimidin-2-yl)cyclopropane-1- carboxamide ESI-MS (M + H)+: 567.2, ¹H NMR (400 MHz, DMSO) δ 10.63 (s, 1H), 8.21 (d, J = 21.5 Hz, 2H), 7.86 (s, 1H), 7.69 (s, 1H), 7.32 (d, J = 11.0 Hz, 2H), 7.06 (s, 1H), 4.90 (d, J = 6.0 Hz, 2H), 4.57 (s, 2H), 2.97 (s, 3H), 2.60 − 2.57 (m, 1H), 2.47 − 2.44 (m, 1H), 2.35 (s, 6H), 1.96 − 1.90 (m, 1H), 1.54 − 1.45 (m, 2H), 0.976- 0.91 (m, 2H), 0.66 − 0.62 (m, 2H). |
| | rac-(1S*,2S*)-N-(6-(((6- | |
| | cyclopropyl-8-(3-methyl-2,4- | |
| | dioxoimidazolidin-1-yl) imidazo | |
| | [1,2-a]pyridin-2-yl)methyl) | |
| | amino)pyrimidin-4-yl)-2-(4,6- | |
| | dimethylpyrimidin-2-yl) cyclo | |
| | propane-1-carboxamide (I-497) | |
| Example 498 | 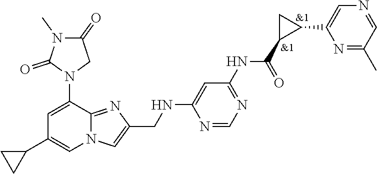 [SMILES: Cc1cncc([C@H]2C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2-(6- methylpyrazin-2-yl)cyclopropane-1- carboxamide ESI-MS (M + H)+: 553.3, ¹H NMR (400 MHz, DMSO) δ 10.64 (s, 1H), 8.51 (s, 1H), 8.35 (s, 1H), 8.24 (s, 1H), 8.18 (s, 1H), 7.89 (s, 1H), 7.68 (s, 1H), 7.32 (d, J = 6.4 Hz, 2H), 4.91 (s, 2H), 4.57 (s, 2H), 2.97 (s, 3H), 2.64 − 2.55 (m, 2H), 2.43 (s, 3H), 1.97 − 1.90 (m, 1H), 1.52 − 1.48 (m, 2H), 0.96 − 0.91 (m, 2H), 0.66 − 0.62 (m, 2H). |
| | rac-(1S*,2S*)-N-(6-(((6- | |
| | cyclopropyl-8-(3-methyl-2,4- | |
| | dioxoimidazolidin-1-yl) | |
| | imidazo[1,2-a]pyridin-2-yl) | |
| | methyl)amino)pyrimidin-4-yl)- | |
| | 2-(6-methylpyrazin-2-yl)cyclo | |
| | propane-1-carboxamide (I-498) | |
| Example 499 | 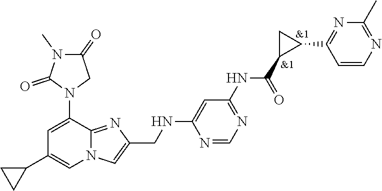 [SMILES: Cc1nccc([C@H]2C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2-(2- methylpyrimidin-4-yl)cyclopropane-1- carboxamide ESI-MS (M + H)+: 553.2, ¹H NMR (400 MHz, DMSO) δ 10.87 (s, 1H), 8.49 (d, J = 5.1 Hz, 1H), 8.37 (d, J = 2.5 Hz, 1H), 8.23 (s, 1H), 7.73 (s, 1H), 7.52 − 7.50 (m, 2H), 7.35 − 7.31 (m, 1H), 7.17 (d, J = 5.1 Hz, 1H), 4.90 (s, 2H), 4.38 (d, J = 5.5 Hz, 2H), 2.95 (s, 3H), 2.64- 2.57 (m, 2H), 2.38 (s, 3H), 1.95 − 1.88 (m, J = 13.3 Hz, 1H), 1.50 − 1.44 (m, J = 13.1 Hz, 2H), 0.93 − 0.89 (m, 2H), 0.64 −0.60 (m, J = 4.7 Hz, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2-(2-methylpyrimidin-4- | |
| | yl)cyclopropane-1-carboxamide | |
| | (I-499) | |
| Example 500 | 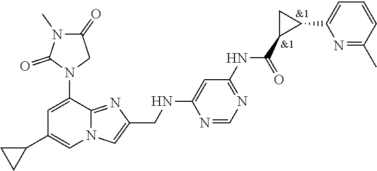 [SMILES: Cc1cccc([C@H]2C[C@@H]2C(=O)Nc2cc(NCc3cn4cc(C5CC5)cc(N5CC(=O)N(C)C5=O)c4n3)ncn2)n1] rac-(1S*,2S*)-N-(6-(((6- | rac-(1S*,2S*)-N-(6-chloropyrimidin-4-yl)-2-(6- methylpyridin-2-yl)cyclopropane-1- carboxamide ESI-MS (M + H)+: 552.3, ¹H NMR (400 MHz, DMSO) δ 10.60 (s, 1H), 8.25 (s, 1H), 8.18 (s, 1H), 7.87 (s, 1H), 7.69 (s, 1H), 7.58 − 7.54 (m, 1H), 7.34 − 7.32 (m, 2H), 7.19 − 7.18 (m, 1H), 7.06 − 7.04 (m, 1H), 4.91 (s, 2H), 4.58 (s, 2H), 2.98 (s, 3H), 2.61 − 2.54 (m, 2H), 2.40 (s, 3H), 1.97 − 1.90 (m, 1H), 1.52 − 1.48 (m, 1H), 1.45 − 1.41 (m, 1H), 0.96 − 0.92 (m, 2H), 0.66 − 0.62 (m, 2H). |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2-(6-methylpyridin-2- | |
| | yl)cyclopropane-1-carboxamide | |
| | (I-500) | |
| Example 501 | 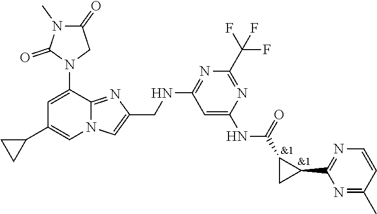 [SMILES: Cc1ccnc([C@H]2C[C@@H]2C(=O)Nc2cc(NCc3cn4cc(C5CC5)cc(N5CC(=O)N(C)C5=O)c4n3)nc(C(F)(F)F)n2)n1] | rac-(1S*,2S*)-N-(6-chloro-2- (trifluoromethyl)pyrimidin-4-yl)-2-(4- methylpyrimidin-2-yl)cyclopropane-1- carboxamide ESI-MS (M + H)+: 621.3, ¹H NMR (400 MHz, DMSO) δ 11.14 (s, 1H), 8.57 − 8.47 (m, 2H), 8.26 (s, 1H), 7.74 (s, 1H), 7.49 (s, 1H), 7.34 (s, 1H), 7.21 (d, J = 5.0 Hz, 1H), 4.93 (d, J = 17.6 Hz, 2H), 4.63-4.44 (m, 2H), 2.98 (s, 3H), 2.67- 2.66 (m, 1H), 2.41 (s, 3H), 2.00 − 1.90 (m, 1H), 1.52-1.51 (m, 2H), 0.97 − 0.92 (m, 2H), 0.67 − 0.62 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-2- | |
| | (trifluoromethyl)pyrimidin-4- | |
| | yl)-2-(4-methylpyrimidin-2- | |
| | yl)cyclopropane-1-carboxamide | |
| | (I-501) | |
| Example 502 | 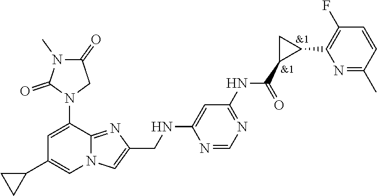 [SMILES: Cc1ccc(F)c([C@H]2C[C@@H]2C(=O)Nc2cc(NCc3cn4cc(C5CC5)cc(N5CC(=O)N(C)C5=O)c4n3)ncn2)n1] | rac-(1S*,2S*)-N-(6-chloropyrimidin-4-yl)-2-(3- fluoro-6-methylpyridin-2-yl)cyclopropane-1- carboxamide ESI-MS (M + H)+: 570.2, ¹H NMR (400 MHz, DMSO) δ 10.64 (s, 1H), 8.21 (d, J = 22.7 Hz, 2H), 7.87 (s, 1H), 7.69 (s, 1H), 7.59 − 7.48 (m, 1H), 7.33 (d, J = 8.8 Hz, 2H), 7.15-7.10 (m, 1H), 4.91 (s, 2H), 4.57 (s, 2H), 2.97 (s, 3H), 2.69-2.64 (m, 1H), 2.62-2.58 (m, 1H), 2.40 (s, 3H), 1.97-1.90 (m, 1H), 1.56-1.46 (m, 2H), 1.00 − 0.86 (m, 2H), 0.68 − 0.56 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-4- | |
| | yl)-2-(3-fluoro-6-methylpyridin- | |
| | 2-yl)cyclopropane-1- | |
| | carboxamide (I-502) | |
| Example 503 | 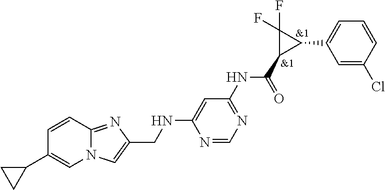 [SMILES: O=C(Nc1cc(NCc2cn3cc(C4CC4)ccc3n2)ncn1)[C@@H]1[C@@H](c2cccc(Cl)c2)C1(F)F] | rac-(1S*,3S*)-3-(3-chlorophenyl)-N-(6- chloropyrimidin-4-yl)-2,2- difluorocyclopropane-1-carboxamide ESI-MS (M + H)+: 495.1, ¹H NMR (400 MHz, DMSO) δ 10.79 (s, 1H), 8.30 (s, 1H), 8.24 (s, 1H), 7.96 (s, 1H), 7.62 (s, 1H), 7.46 − 7.40 (m, 3H), 7.37 (d, J = 9.3 Hz, 1H), 7.33 − 7.22 (m, 2H), 6.98-6.93(m, 1H), 4.59 (s, 2H), 3.71 − 3.63 (m, 1H), 3.55-3.47 (m, 1H), 1.95-1.86 (m, 1H), 0.95 − 0.87 (m, 2H), 0.69 − 0.62 (m, 2H). |
| | rac-(1S*,3S*)-3-(3- | |
| | chlorophenyl)-N-(6-(((6- | |
| | cyclopropylimidazo[1,2- | |
| | a]pyridin-2- | |
| | yl)methyl)amino)pyrimidin-4- | |
| | yl)-2,2-difluorocyclopropane-1- | |
| | carboxamide (I-503) | |
| Example 504 | 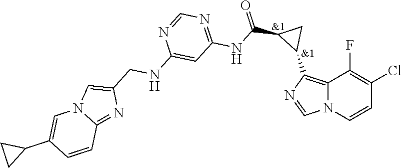 [SMILES: O=C(Nc1cc(NCc2cn3cc(C4CC4)ccc3n2)ncn1)[C@H]1C[C@@H]1c1ncn2ccc(Cl)c(F)c12] | rac-(1S*,2S*)-2-(7-chloro-8-fluoroimidazo[1,5- a]pyridin-1-yl)-N-(6-chloropyrimidin-4- yl)cyclopropane-1-carboxamide ESI-MS (M + H)+: 517.1, ¹H NMR (400 MHz, DMSO) δ 10.63 (s, 1H), 8.39 (d, J = 2.3 Hz, 1H), 8.30 (s, 1H), 8.23 − 8.12 (m, 2H), 7.85 (s, 1H), 7.61 (s, 1H), 7.37 (d, J = 9.3 Hz, 1H), 7.29 (s, 1H), 6.96 (dd, J = 9.3, 1.6 Hz, 1H), 6.76 − 6.69 (m, 1H), 4.57 (s, 2H), 2.67-2.66 (m, 1H), 2.49-2.47 (m, 1H), 1.94 − 1.87 (m, 1H), 1.55 − 1.45 (m, 2H), 0.92-0.89 (m, 2H), 0.67-0.65 (m, 2H). |
| | rac-(1S*,2S*)-2-(7-chloro-8- | |
| | fluoroimidazo[1,5-a]pyridin-1- | |
| | yl)-N-(6-(((6- | |
| | cyclopropylimidazo[1,2- | |
| | a]pyridin-2- | |
| | yl)methyl)amino)pyrimidin-4- | |
| | yl)cyclopropane-1-carboxamide | |
| | (I-504) | |
| Example 505 | 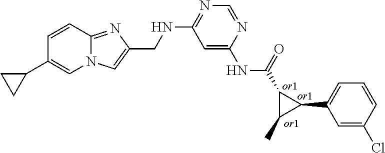 [SMILES: C[C@H]1[C@H](C(=O)Nc2cc(NCc3cn4cc(C5CC5)ccc4n3)ncn2)[C@H]1c1cccc(Cl)c1] (1R*,2R*,3S*)-2-(3- | (1R*,2R*,3S*)-2-(3-chlorophenyl)-N-(6- chloropyrimidin-4-yl)-3-methylcyclopropane-1- carboxamide, isomer 1 − 4 ESI-MS (M + H)+: 473.2, ¹H NMR (400 MHz, DMSO) δ 10.62 (s, 1H), 8.30 (s, 1H), 8.22 (s, 1H), 7.86 (s, 1H), 7.61 (s, 1H), 7.39-7.34 (m, 3H), 7.33 − 7.21 (m, 3H), 6.98-6.93 (m, 1H), 4.57 (s, 2H), 2.62-2.56 (m, 1H), 2.42 (t, J = 4.5 Hz, 1H), 1.95-1.86 (m, 1H), 1.76 − 1.67 (m, 1H), 0.94 − 0.88 (m, 2H), 0.83 (d, J = 6.3 Hz, 3H), 0.69 − 0.62 (m, 2H). |
| | chlorophenyl)-N-(6-(((6- | |
| | cyclopropylimidazo[1,2- | |
| | a]pyridin-2- | |
| | yl)methyl)amino)pyrimidin-4- | |
| | yl)-3-methylcyclopropane-1- | |
| | carboxamide, isomer 2 (I-505) | |
| Example 506 | 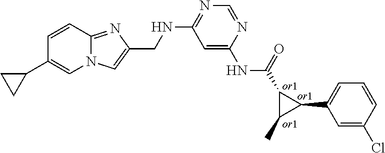 [SMILES: C[C@H]1[C@H](C(=O)Nc2cc(NCc3cn4cc(C5CC5)ccc4n3)ncn2)[C@H]1c1cccc(Cl)c1] (1R*,2R*,3S*)-2-(3- chlorophenyl)-N-(6-(((6- cyclopropylimidazo[1,2- a]pyridin-2- | (1R*,2R*,3S*)-2-(3-chlorophenyl)-N-(6- chloropyrimidin-4-yl)-3-methylcyclopropane-1- carboxamide, isomer 1 − 4 ESI-MS (M + H)+: 473.2, ¹H NMR (400 MHz, DMSO) δ 10.61-10.45 (m, 1H), 8.26 (d, J = 5.2 Hz, 1H), 8.19 (s, 1H), 8.15 (d, J = 3.7 Hz, 1H), 7.70 (s, 1H), 7.56 (d, J = 9.3 Hz, 1H), 7.34 (d, J = 9.3 Hz, 1H), 7.30 − 7.20 (m, 2H), 7.16 (d, J = 9.7 Hz, 2H), 7.12 − 7.02 (m, 1H), 6.96-6.94 (m, 1H), 4.54 (s, 2H), 2.62 (t, J = 8.9 Hz, 1H), 2.42- 2.34 (m, 1H), 2.23-2.18 (m, 0.5H), 2.04-1.97 (m, 0.5H), 1.94 − 1.86 (m, 1H), 1.82 − 1.71 (m, 1H), 1.23 (d, J = 6.3 Hz, 3H), 0.93 − 0.87 (m, 2H), 0.69-0.61 (m, 2H). |
| | yl)methyl)amino)pyrimidin-4- | |
| | yl)-3-methylcyclopropane-1- | |
| | carboxamide, isomer 3 (I-506) | |
| Example 507 | 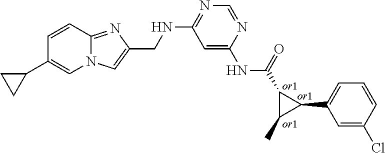 [SMILES: C[C@H]1[C@H](C(=O)Nc2cc(NCc3cn4cc(C5CC5)ccc4n3)ncn2)[C@H]1c1cccc(Cl)c1] (1R*,2R*,3S*)-2-(3- chlorophenyl)-N-(6-(((6- | (1R*,2R*,3S*)-2-(3-chlorophenyl)-N-(6- chloropyrimidin-4-yl)-3-methylcyclopropane-1- carboxamide, isomer 1 − 4 ESI-MS (M + H)+: 473.2, ¹H NMR (400 MHz, DMSO) δ 10.50 (s, 1H), 8.29 (s, 1H), 8.19 (s, 1H), 7.85 (s, 1H), 7.61 (s, 1H), 7.37 (d, J = 9.3 Hz, 1H), 7.34 − 7.27 (m, 2H), 7.24 (d, J = 8.6 Hz, 1H), 7.19 (s, 1H), 7.10 (d, J = 7.6 Hz, 1H), 6.96 (d, J = 9.3 Hz, 1H), 4.57 (s, 2H), 2.47-2.41 (m, 1H), 2.38 (t, J = 5.6 Hz, 1H), 1.95-1.86 (m, 1H), 1.81 − 1.72 (m, 1H), 1.27 (d, J = 6.0 Hz, 3H), 0.94 − 0.87 (m, 2H), 0.69 − 0.63 (m, 2H). |
| | cyclopropylimidazo[1,2- | |
| | a]pyridin-2- | |
| | yl)methyl)amino)pyrimidin-4- | |
| | yl)-3-methylcyclopropane-1- | |
| | carboxamide, isomer 1 (I-507) | |
| Example 508 | 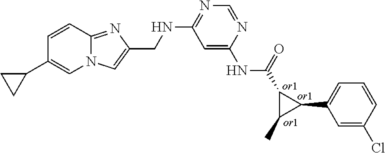 [SMILES: C[C@H]1[C@H](C(=O)Nc2cc(NCc3cn4cc(C5CC5)ccc4n3)ncn2)[C@H]1c1cccc(Cl)c1] (1R*,2R*,3S*)-2-(3- chlorophenyl)-N-(6-(((6- | (1R*,2R*,3S*)-2-(3-chlorophenyl)-N-(6- chloropyrimidin-4-yl)-3-methylcyclopropane-1- carboxamide, isomer 1 − 4 ESI-MS (M + H)+: 473.2, ¹H NMR (400 MHz, DMSO) δ 10.47 (s, 1H), 8.26 (s, 1H), 8.15 (s, 1H), 7.74 (s, 1H), 7.55 (s, 1H), 7.34 (d, J = 9.3 Hz, 1H), 7.26 (s, 1H), 7.22 (d, J = 7.6 Hz, 1H), 7.20 − 7.15 (m, 2H), 7.04 (s, 1H), 6.94 (d, J = 9.3 Hz, 1H), 4.50 (s, 2H), 2.42 − 2.36 (m, 1H), 2.23-2.18 (m, 1H), 2.04-1.97 (m, 1H), 1.94-1.85 (m, 1H), 1.23 (d, J = 6.0 Hz, 3H), 0.93 − 0.86 (m, 2H), 0.68 − 0.62 (m, 2H). |
| | cyclopropylimidazo[1,2- | |
| | a]pyridin-2- | |
| | yl)methyl)amino)pyrimidin-4- | |
| | yl)-3-methylcyclopropane-1- | |
| | carboxamide, isomer 4 (I-508) | |
| Example 509 | 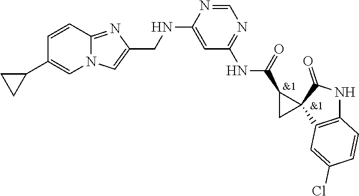 [SMILES: O=C(Nc1cc(NCc2cn3cc(C4CC4)ccc3n2)ncn1)[C@@H]1C[C@]12C(=O)Nc1ccc(Cl)cc12] | rac-(1R*,2S*)-5′-chloro-N-(6-chloropyrimidin- 4-yl)-2′-oxospiro[cyclopropane-1,3′-indoline]-2- carboxamide ESI-MS (M + H)+: 500.2, ¹H NMR (400 MHz, DMSO) δ 10.63 (d, J = 6.7 Hz, 2H), 8.29 (s, 1H), 8.14 (s, 1H), 7.88 (s, 1H), 7.60 (s, 1H), 7.37 (d, J = 9.3 Hz, 1H), 7.29 − 7.17 (m, 2H), 7.14 (d, J = 1.7 Hz, 1H), 6.95 (d, J = 9.3 Hz, 1H), 6.87 (d, J = 8.2 Hz, 1H), 4.57 (s, 2H), 3.03 (t, J = 8.4 Hz, 1H), 2.10 − 2.00 (m, 1H), 1.97 − 1.80 (m, 2H), 0.93 − 0.86 (m, 2H), 0.72 − 0.55 (m, 2H). |
| | rac-(1R*,2S*)-5′-chloro-N-(6- | |
| | (((6-cyclopropylimidazo[1,2- | |
| | a]pyridin-2- | |
| | yl)methyl)amino)pyrimidin-4- | |
| | yl)-2′-oxospiro[cyclopropane- | |
| | 1,3′-indoline]-2-carboxamide (I- | |
| | 509) | |
| Example 510 | 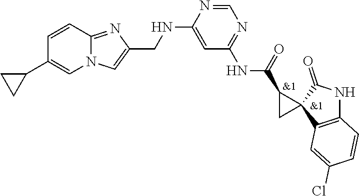 [SMILES: O=C(Nc1cc(NCc2cn3cc(C4CC4)ccc3n2)ncn1)[C@@H]1C[C@@]12C(=O)Nc1ccc(Cl)cc12] | rac-(1R*,2R*)-5′-chloro-N-(6-chloropyrimidin- 4-yl)-2′-oxospiro[cyclopropane-1,3′-indoline]-2- carboxamide ESI-MS (M + H)+: 500.1, ¹H NMR (400 MHz, DMSO) δ 10.87 (s, 1H), 10.76 (s, 1H), 8.48 (s, 1H), 8.28 (s, 1H), 8.16 (s, 1H), 7.97 (s, 1H), 7.61 (s, 1H), 7.37 − 7.35 (m, 1H), 7.28 − 7.21 (m, 3H), 6.96 − 6.90 (m, 2H), 4.56 (s, 2H), 2.98 (t, J = 8.0 Hz, 1H), 2.14 − 2.11 (m, 1H), 1.93 − 1.87(m, 1H), 1.85 − 1.82 (m, 1H), 0.93 − 0.88 (m, 2H), 0.67 − 0.63 (m, 2H). |
| | rac-(1R*,2R*)-5′-chloro-N-(6- | |
| | (((6-cyclopropylimidazo[1,2- | |
| | a]pyridin-2- | |
| | yl)methyl)amino)pyrimidin-4- | |
| | yl)-2′-oxospiro[cyclopropane- | |
| | 1,3′-indoline]-2-carboxamide (I- | |
| | 510) | |
| Example 511 | 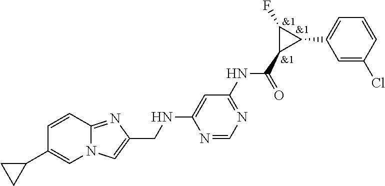 [SMILES: O=C(Nc1cc(NCc2cn3cc(C4CC4)ccc3n2)ncn1)[C@H]1[C@H](F)[C@@H]1c1cccc(Cl)c1] | rac-(1R*,2R*,3S*)-2-(3-chlorophenyl)-N-(6- chloropyrimidin-4-yl)-3-fluorocyclopropane-1- carboxamide ESI-MS (M + H)+: 477.1, ¹H NMR (400 MHz, DMSO) δ 10.75 (s, 1H), 8.29 (s, 1H), 8.23 (s, 1H), 7.92 (s, 1H), 7.61 (s, 1H), 7.43-7.33 (m, 4H), 7.30 (d, J = 7.2 Hz, 1H), 7.23 (s, 1H), 6.95 (d, J = 9.3 Hz, 1H), 5.23-5.7 (m, 1H), 4.57 (s, 2H), 3.14-3.06 (m, 1H), 2.84-2.77 (m, 1H), 1.94 − 1.87 (m, 1H), 0.95 − 0.87 (m, 2H), 0.69-0.62 (m, 2H). |
| | rac-(1R*,2R*,3S*)-2-(3- | |
| | chlorophenyl)-N-(6-(((6- | |
| | cyclopropylimidazo[1,2- | |
| | a]pyridin-2- | |
| | yl)methyl)amino)pyrimidin-4- | |
| | yl)-3-fluorocyclopropane-1- | |
| | carboxamide (I-511) | |
| Example 512 | 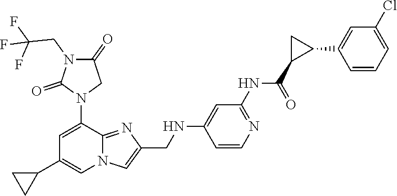 [SMILES: O=C(Nc1cc(NCc2cn3cc(C4CC4)cc(N4CC(=O)N(CC(F)(F)F)C4=O)c3n2)ccn1)[C@H]1C[C@@H]1c1cccc(Cl)c1] | (1S,2S)-2-(3-chlorophenyl)-N-(4-fluoropyridin- 2-yl)cyclopropane-1-carboxamide ESI-MS [M + H] ⁺: 638.1, ¹H NMR (400 MHz, cd3od) δ 8.17 (s, 1H), 7.75 (d, J = 6.0 Hz, 1H), 7.71 (s, 1H), 7.35 (s, 1H), 7.31 (d, J = 0.9 Hz, 1H), 7.27 − 7.23(m, 1H), 7.18 (d, J = 7.1 Hz, 2H), 7.09 (d, J = 7.6 Hz, 1H), 6.39 (dd, J = 6.0, 2.1 Hz, 1H), 4.80 (s, 2H), 4.49 (s, 2H), 4.31 (q, J = 8.9 Hz, 2H), 2.49 − 2.44 (m, 1H), 2.18 − 2.13 (m, 1H), 1.99 − 1.92 (m, 1H), 1.62 − 1.57 (m, 2H), 1.00 − 0.95 (m, 2H), 0.75 − 0.71 (m,, 2H). |
| | (1S,2S)-2-(3-chlorophenyl)-N- | |
| | (4-(((6-cyclopropyl-8-(2,4- | |
| | dioxo-3-(2,2,2- | |
| | trifluoroethyl)imidazolidin-1- | |
| | yl)imidazo[1,2-a]pyridin-2- | |
| | yl)methyl)amino)pyridin-2- | |
| | yl)cyclopropane-1-carboxamide | |
| | (I-512) | |
| Example 513 | 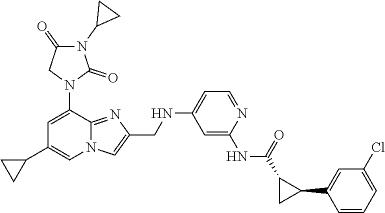 [SMILES: O=C(Nc1cc(NCc2cn3cc(C4CC4)cc(N4CC(=O)N(C5CC5)C4=O)c3n2)ccn1)[C@H]1C[C@@H]1c1cccc(Cl)c1] | (1S,2S)-2-(3-chlorophenyl)-N-(4-fluoropyridin- 2-yl)cyclopropane-1-carboxamide ESI-MS [M + H]+: 596.2, ¹H NMR (400 MHz, MeOD) δ 8.15 (d, J = 0.9 Hz, 1H), 7.75 (d, J = 6.0 Hz, 1H), 7.70 (s, 1H), 7.38 (s, 1H), 7.27 − 7.23 (m, 2H), 7.20 − 7.17 (m, 2H), 7.10 (d, J = 7.6 Hz, 1H), 6.38 (dd, J = 6.0, 2.2 Hz, 1H), 4.59 (s, 2H), 4.48 (s, 2H), 2.70 − 2.65 (m, 1H), 2.49 − 2.44 (m, 1H), 2.19 − 2.13 (m, 1H), 1.98 − 1.91 (m, 1H), 1.62 − 1.57 (m, 1H), 1.36 − 1.34 (m, 1H), 1.03 − 0.93 (m, 6H), 0.74 − 0.70 (m, 2H). |
| | (1S,2S)-2-(3-chlorophenyl)-N- | |
| | (4-(((6-cyclopropyl-8-(3- | |
| | cyclopropyl-2,4- | |
| | dioxoimidazolidin-1- | |
| | yl)imidazo[1,2-a]pyridin-2- | |
| | yl)methyl)amino)pyridin-2- | |
| | yl)cyclopropane-1-carboxamide | |
| | (I-513) | |
| Example 514 | 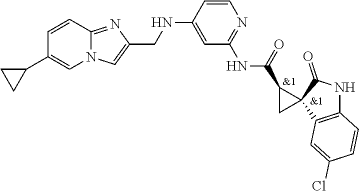 [SMILES: O=C(Nc1cc(NCc2cn3cc(C4CC4)ccc3n2)ccn1)[C@@H]1C[C@]12C(=O)Nc1ccc(Cl)cc12] rac-(1R*,2S*)-5′-chloro-N-(4- | rac-(1R*,2S*)-5′-chloro-N-(4-fluoropyridin-2- yl)-2′-oxospiro[cyclopropane-1,3′-indoline]-2- carboxamide ESI-MS [M + H] ⁺: 499.1. ¹H NMR (400 MHz, DMSO) δ 10.85 (s, 1H), 10.48 (s, 1H), 8.28 (s, 1H), 7.72 (d, J = 5.7 Hz, 1H), 7.63 (s, 1H), 7.41 7.37 (m, 2H), 7.28 (d, J = 1.7 Hz, 1H), 7.22 − 7.14 (m, 2H), 6.97 (dd, J = 9.3, 1.6 Hz, 1H), 6.90 (d, J = 8.3 Hz, 1H), 6.30 (dd, J = 5.7, 1.9 Hz, 1H), 5.32 (t, J = 4.7 Hz, 1H), 4.35 (d, J = 5.7 Hz, 2H), 2.98 − 2.94 (m, 1H), 2.14 − 2.11 (m, 1H), 1.92 − 1.87 (m, 1H), 1.82 − 1.79 (m, 1H), 0.93 − 0.89 (m, 2H), 0.68 − 0.64 (m, 2H). |
| | (((6-cyclopropylimidazo[1,2- | |
| | a]pyridin-2- | |
| | yl)methyl)amino)pyridin-2-yl)- | |
| | 2′-oxospiro[cyclopropane-1,3′- | |
| | indoline]-2-carboxamide (I-514) | |
| Example 515 | 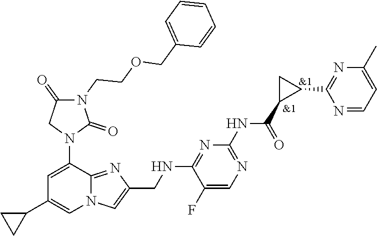 [SMILES: Cc1ccnc([C@H]2C[C@@H]2C(=O)Nc2ncc(F)c(NCc3cn4cc(C5CC5)cc(N5CC(=O)N(CCOCc6ccccc6)C5=O)c4n3)n2)n1] | rac-(1S*,2S*)-N-(4-chloro-5-fluoropyrimidin- 2-yl)-2-(4-methylpyrimidin-2-yl)cyclopropane- 1-carboxamide ESI-MS [M + H]+: 691.3. ¹H NMR (400 MHz, DMSO) δ 10.42 (s, 1H), 8.46 (d, J = 5.1 Hz, 1H), 8.22 (d, J = 4.9 Hz, 2H), 8.00 (d, J = 3.4 Hz, 1H), 7.79 (s, 1H), 7.32 (s, 6H), 7.14 (d, J = 5.1 Hz, 1H), 4.92 (s, 2H), 4.61 (d, J = 5.6 Hz, 2H), 4.51 (s, 2H), 3.72 (t, J = 5.3 Hz, 2H), 3.64 (t, J = 5.1 Hz, 2H), 2.87-2.86 (m, 1H), 2.51-2.50 (m, 1H), 2.37 (s, 3H), 1.97 − 1.90 (m, 1H), 1.52-1.49 (m, 2H), 0.95-0.93 (m, 2H), 0.65-0.63 (m, 2H). |
| | rac-(1S*,2S*)-N-(4-(((8-(3-(2- | |
| | (benzyloxy)ethyl)-2,4- | |
| | dioxoimidazolidin-1-yl)-6- | |
| | cyclopropylimidazo[1,2- | |
| | a]pyridin-2-yl)methyl)amino)-5- | |
| | fluoropyrimidin-2-yl)-2-(4- | |
| | methylpyrimidin-2- | |
| | yl)cyclopropane-1-carboxamide | |
| | (I-515) | |
| Example 516 | 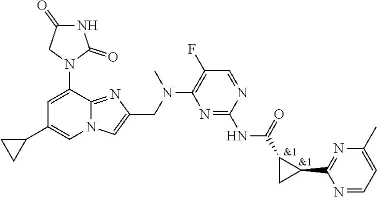 [SMILES: Cc1ccnc([C@H]2C[C@@H]2C(=O)Nc2ncc(F)c(N(C)Cc3cn4cc(C5CC5)cc(N5CC(=O)NC5=O)c4n3)n2)n1] | rac-(1S*,2S*)-N-(4-chloro-5-fluoropyrimidin- 2-yl)-2-(4-methylpyrimidin-2-yl)cyclopropane- 1-carboxamide ESI-MS [M + H]+: 571.2. ¹H NMR (400 MHz, DMSO) δ 11.33 (s, 1H), 10.50 (s, 1H), 8.48 (d, J = 5.1 Hz, 1H), 8.19 (s, 1H), 8.06 (d, J = 6.6 Hz, 1H), 7.77 (s, 1H), 7.36 (d, J = 1.1 Hz, 1H), 7.17 (d, J = 5.1 Hz, 1H), 4.83 (s, 4H), 3.21 (s, 3H), 2.83 − 2.82 (m, 1H), 2.54-2.53 (m, 1H), 2.39 (s, 3H), 1.94-1.92 (m, 1H), 1.54 − 1.48 (m, 2H), 0.95 − 0.91 (m, 2H), 0.66 − 0.61 (m, 2H). |
| | rac-(1S*,2S*)-N-(4-(((6- | |
| | cyclopropyl-8-(2,4- | |
| | dioxoimidazolidin-1- | |
| | yl)imidazo[1,2-a]pyridin-2- | |
| | yl)methyl)(methyl)amino)-5- | |
| | fluoropyrimidin-2-yl)-2-(4- | |
| | methylpyrimidin-2- | |
| | yl)cyclopropane-1-carboxamide | |
| | (I-516) | |
| Example 517 | 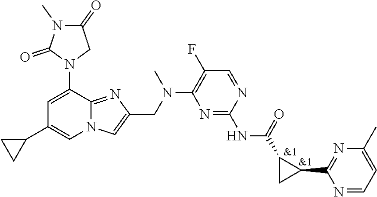 | rac-(1S*,2S*)-N-(4-chloro-5-fluoropyrimidin- 2-yl)-2-(4-methylpyrimidin-2-yl)cyclopropane- 1-carboxamide ESI-MS [M + H]+: 571.2. ¹H NMR (400 MHz, DMSO) δ 10.42 (s, 1H), 8.46 (d, J = 5.1 Hz, 1H), 8.20 (d, J = 6.1 Hz, 2H), 8.01 (d, J = 3.4 Hz, 1H), 7.79 (s, 1H), 7.32 (d, J = 1.5 Hz, 1H), 7.14 (d, J = 5.1 Hz, 1H), 4.88 (s, 2H), 4.61 (d, J = 5.9 Hz, 2H), 2.97 (s, 3H), 2.86 (s, 2H), 2.37 (s, 3H), 1.94 − 1.91 (m, 1H), 1.55 − 1.46 (m, 2H), 0.97 − 0.88 (m, 2H), 0.64 − 0.60 (m, 2H). |
| | rac-(1S*,2S*)-N-(4-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-5- | |
| | fluoropyrimidin-2-yl)-2-(4- | |
| | methylpyrimidin-2- | |
| | yl)cyclopropane-1-carboxamide | |
| | (I-517) | |
| Example 518 | 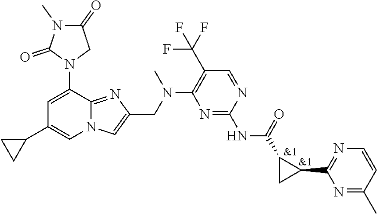 | rac-(1S*,2S*)-N-(4-chloro-5- (trifluoromethyl)pyrimidin-2-yl)-2-(4- methylpyrimidin-2-yl)cyclopropane-1- carboxamide ESI-MS [M + H]+: 621.2. ¹H NMR (400 MHz, DMSO) δ 10.73 (s, 1H), 8.46 (d, J = 5.1 Hz, 1H), 8.35 (s, 1H), 8.23 (s, 1H), 7.87-7.84 (m, 1H), 7.71 (s, 1H), 7.34 (s, 1H), 7.14 (d, J = 5.1 Hz, 1H), 4.87 (s, 2H), 4.70 (d, J = 5.4 Hz, 2H), 2.97 (s, 3H), 2.58-2.55 (m, 2H), 2.38 (s, 3H), 1.99- 1.90 (m, 1H), 1.53 (s, 2H), 0.96-0.91 (m, 2H), 0.66-0.64 (m, 2H). |
| | rac-(1S*,2S*)-N-(4-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-5- | |
| | (trifluoromethyl)pyrimidin-2- | |
| | yl)-2-(4-methylpyrimidin-2- | |
| | yl)cyclopropane-1-carboxamide | |
| | (I-518) | |
| Example 519 | 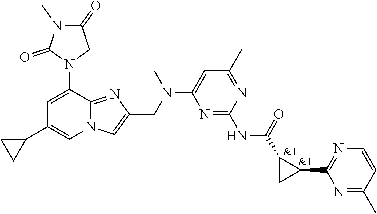 | rac-(1S*,2S*)-N-(4-chloro-6-methylpyrimidin- 2-yl)-2-(4-methylpyrimidin-2-yl)cyclopropane- 1-carboxamide ESI-MS [M + H]+: 567.2. ¹H NMR (400 MHz, DMSO) δ 1H NMR (400 MHz, DMSO) δ 10.17 (s, 1H), 8.46 (d, J = 4.9 Hz, 1H), 8.24 (d, J = 1.0 Hz, 1H), 7.73 (s, 2H), 7.32 (d, J = 1.2 Hz, 1H), 7.12 (d, J = 4.4 Hz, 1H), 6.09 (s, 1H), 4.89 (s, 2H), 4.50 (s, 2H), 2.97 (s, 3H), 2.52-2.51 (m, 2H), 2.39 − 2.33 (m, 3H), 2.09 (s, 3H), 1.96 − 1.91 (m, 1H), 1.55 − 1.48 (m, 2H), 0.99-0.91 (m, 2H), 0.67 − 0.62 (m, 2H). |
| | rac-(1S*,2S*)-N-(4-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-6- | |
| | methylpyrimidin-2-yl)-2-(4- | |
| | methylpyrimidin-2- | |
| | yl)cyclopropane-1-carboxamide | |
| | (I-519) | |
| Example 520 | 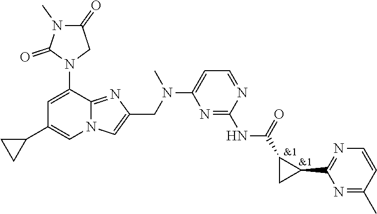 | rac-(1S*,2S*)-N-(4-chloropyrimidin-2-yl)-2-(4- methylpyrimidin-2-yl)cyclopropane-1- carboxamide ESI-MS [M + H]+: 553.2. ¹H NMR (400 MHz, DMSO) δ 10.23 (s, 1H), 8.50-8.46 (m, 1H), 8.26- 8.22 (m, 2H), 7.89 (s, 1H), 7.76 (s, 1H), 7.33 (s, 1H), 7.18 − 7.09 (m, 1H), 6.24 (d, J = 5.9 Hz, 1H), 4.89 (s, 2H), 4.51 (s, 2H), 2.97 (d, J = 4.0 Hz, 3H), 2.54 (s, 2H), 2.38 (d, J = 11.7 Hz, 3H), 1.94 (s, 1H), 1.52 (s, 2H), 0.97 − 0.90 (m, 2H), 0.65-0.63 (m, 2H). |
| | rac-(1S*,2S*)-N-(4-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrimidin-2- | |
| | yl)-2-(4-methylpyrimidin-2- | |
| | yl)cyclopropane-1-carboxamide | |
| | (I-520) | |
| Example 521 | 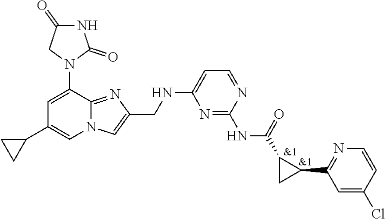 [SMILES: O=C1CN(c2cc(C3CC3)cn3cc(CNc4ccnc(NC(=O)[C@H]5C[C@@H]5c5cc(Cl)ccn5)n4)nc23)C(=O)N1] | rac-(1S*,2S*)-2-(4-chloropyridin-2-yl)-N-(4- chloropyrimidin-2-yl)cyclopropane-1- carboxamide ESI-MS [M + H]+: 558.1. ¹H NMR (400 MHz, DMSO) δ 10.21 (s, 1H), 8.36 (d, J = 5.5 Hz, 1H), 8.22 (s, 1H), 7.95 − 7.81 (m, 3H), 7.74 (s, 1H), 7.63 (d, J = 1.8 Hz, 1H), 7.31 (s, 1H), 7.30 − 7.26 (m, 1H), 6.23 (d, J = 5.9 Hz, 1H), 5.32 (s, 1H), 4.86 (s, 2H), 4.51 (s, 2H), 2.69 − 2.66 (m, 1H), 2.64 − 2.58 (m, 1H), 2.34 − 2.31 (m, 1H), 1.99 − 1.92 (m, 2H), 0.96 − 0.91 (m, 2H), 0.67 − 0.62 (m, 2H). |
| | rac-(1S*,2S*)-2-(4- | |
| | chloropyridin-2-yl)-N-(4-(((6- | |
| | cyclopropyl-8-(2,4- | |
| | dioxoimidazolidin-1- | |
| | yl)imidazo[1,2-a]pyridin-2- | |
| | yl)methyl)amino)pyrimidin-2- | |
| | yl)cyclopropane-1-carboxamide | |
| | (I-521) | |
| Example 522 | 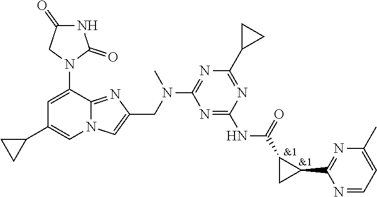 [SMILES: Cc1ccnc([C@H]2C[C@@H]2C(=O)Nc2nc(C3CC3)nc(N(C)Cc3cn4cc(C5CC5)cc(N5CC(=O)NC5=O)c4n3)n2)n1] | rac-(1S*,2S*)-N-(4-chloro-6-cyclopropyl- 1,3,5-triazin-2-yl)-2-(4-methylpyrimidin-2- yl)cyclopropane-1-carboxamide ESI-MS [M + H]+: 594.2 ¹H NMR (400 MHz, DMSO) δ 11.35 (s, 1H), 10.50 (s, 1H), 8.52-8.41 (m, 1H), 8.19 (d, J = 13.1 Hz, 1H), 7.68 (d, J = 15.2 Hz, 1H), 7.34 (s, 1H), 7.35-7.10 (m, 1H), 4.85 − 4.72 (m, 4H), 3.10 (d, J = 35.5 Hz, 3H), 2.53 (d, J = 8.1 Hz, 2H), 2.37 (d, J = 17.1 Hz, 3H), 1.95-1.92 (m, 1H), 1.79-1.77 (m, 1H), 1.64 − 1.46 (m, 2H), 0.99-0.90(m, 6H), 0.65-0.62 (m, 2H). |
| | rac-(1S*,2S*)-N-(4- | |
| | cyclopropyl-6-(((6-cyclopropyl- | |
| | 8-(2,4-dioxoimidazolidin-1- | |
| | yl)imidazo[1,2-a]pyridin-2- | |
| | yl)methyl)(methyl)amino)-1,3,5- | |
| | triazin-2-yl)-2-(4- | |
| | methylpyrimidin-2- | |
| | yl)cyclopropane-1-carboxamide | |
| | (I-522) | |
| Example 523 | 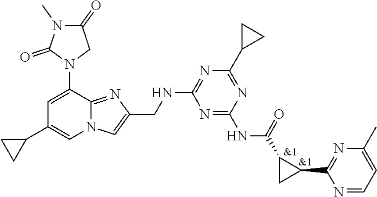 [SMILES: Cc1ccnc([C@H]2C[C@@H]2C(=O)Nc2nc(NCc3cn4cc(C5CC5)cc(N5CC(=O)N(C)C5=O)c4n3)nc(C3CC3)n2)n1] | rac-(1S*,2S*)-N-(4-chloro-6-cyclopropyl- 1,3,5-triazin-2-yl)-2-(4-methylpyrimidin-2- yl)cyclopropane-1-carboxamide ESI-MS [M + H]+: 594.2. ¹H NMR (400 MHz, DMSO) δ 10.46 (d, J = 22.1 Hz, 1H), 8.52-8.41 (m, 1H), 8.29 − 7.97 (m, 2H), 7.69 (d, J = 22.8 Hz, 1H), 7.32 (d, J = 1.4 Hz, 1H), 7.22-7.10 (m, 1H), 4.89 (d, J = 3.3 Hz, 2H), 4.58-4.46 (m, 2H), 2.97 (s, 3H), 2.60 − 2.50 (m, 2H), 2.39 (d, J = 20.3 Hz, 3H), 2.01 − 1.84 (m, 1H), 1.76-1.72 (m, 1H), 1.58-1.52 (m, 2H), 0.98-0.87 (m, 6H), 0.67- 0.61 (m, 2H). |
| | rac-(1S*,2S*)-N-(4- | |
| | cyclopropyl-6-(((6-cyclopropyl- | |
| | 8-(3-methyl-2,4- | |
| | dioxoimidazolidin-1- | |
| | yl)imidazo[1,2-a]pyridin-2- | |
| | yl)methyl)amino)-1,3,5-triazin- | |
| | 2-yl)-2-(4-methylpyrimidin-2- | |
| | yl)cyclopropane-1-carboxamide | |
| | (I-523) | |
| Example 524 | 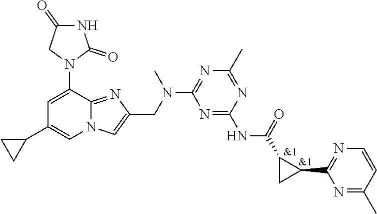 [SMILES: Cc1ccnc([C@H]2C[C@@H]2C(=O)Nc2nc(C)nc(N(C)Cc3cn4cc(C5CC5)cc(N5CC(=O)NC5=O)c4n3)n2)n1] | rac-(1S*,2S*)-N-(4-chloro-6-methyl-1,3,5- triazin-2-yl)-2-(4-methylpyrimidin-2- yl)cyclopropane-1-carboxamide ESI-MS [M + H]+: 568.2 ¹H NMR (400 MHz, DMSO)) δ 11.33 (s, 1H), 10.62 (d, J = 6.6 Hz, 1H), 8.52-8.40 (m, 1H), 8.18 (s, 1H), 7.69 (d, J = 2.8 Hz, 1H), 7.34 (s, 1H), 7.20-7.06(m, 1H), 4.94 − 4.63 (m, 4H), 3.10 (d, J = 43.9 Hz, 3H), 2.53- 2.50 (m, 2H), 2.36 (d, J = 20.2 Hz, 3H), 2.24 (s, 3H), 1.95-1.90 (m, 1H), 1.58-1.53 (m, 2H), 0.98 − 0.87 (m, 2H), 0.69 − 0.55 (m, 2H). |
| | rac-(1S*,2S*)-N-(4-(((6- | |
| | cyclopropyl-8-(2,4- | |
| | dioxoimidazolidin-1- | |
| | yl)imidazo[1,2-a]pyridin-2- | |
| | yl)methyl)(methyl)amino)-6- | |
| | methyl-1,3,5-triazin-2-yl)-2-(4- | |
| | methylpyrimidin-2- | |
| | yl)cyclopropane-1-carboxamide | |
| | (I-524) | |
| Example 525 | 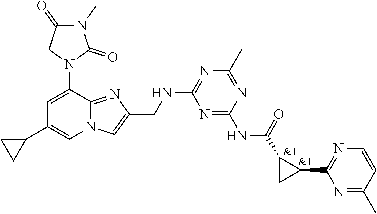 [SMILES: Cc1ccnc([C@H]2C[C@@H]2C(=O)Nc2nc(C)nc(NCc3cn4cc(C5CC5)cc(N5CC(=O)N(C)C5=O)c4n3)n2)n1] | rac-(1S*,2S*)-N-(4-chloro-6-methyl-1,3,5- triazin-2-yl)-2-(4-methylpyrimidin-2- yl)cyclopropane-1-carboxamide ESI-MS [M + H]+: 568.2. ¹H NMR (400 MHz, DMSO) δ 10.61 (d, J = 30.0 Hz, 1H), 8.54- 8.49(m, 1H), 8.35 − 8.14 (m, 2H), 7.70 (d, J = 11.0 Hz, 1H), 7.32 (s, 1H), 7.25 −7.08(m, 1H), 4.86 (d, J = 18.1 Hz, 2H), 4.62 −4.47(m, 1H), 2.97 (s, 3H), 2.58-2.55 (m, 1H), 2.38 (d, J = 24.9 Hz, 3H), 2.20 (d, J = 4.8 Hz, 3H), 1.97 − 1.88 (m, 1H), 1.59 − 1.47 (m, 2H), 0.98 − 0.88 (m, 2H), 0.69 − 0.59 (m, 2H). |
| | rac-(1S*,2S*)-N-(4-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)-6-methyl- | |
| | 1,3,5-triazin-2-yl)-2-(4- | |
| | methylpyrimidin-2- | |
| | yl)cyclopropane-1-carboxamide | |
| | (I-525) | |
| Example 526 | 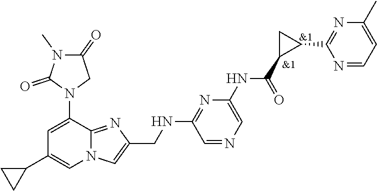 [SMILES: Cc1ccnc([C@H]2C[C@@H]2C(=O)Nc2cncc(NCc3cn4cc(C5CC5)cc(N5CC(=O)N(C)C5=O)c4n3)n2)n1] | rac-(1S*,2S*)-N-(6-chloropyrazin-2-yl)-2-(4- methylpyrimidin-2-yl)cyclopropane-1- carboxamide ESI-MS [M + H]+: 553.2. ¹H NMR (400 MHz, DMSO) δ 10.55 (s, 1H), 8.52 (d, J = 5.1 Hz, 1H), 8.44 (s, 1H), 8.24 (d, J = 1.1 Hz, 1H), 7.73 (d, J = 8.9 Hz, 2H), 7.55-7.52 (m, 1H), 7.32 (d, J = 1.5 Hz, 1H), 7.20 (d, J = 5.1 Hz, 1H), 4.88 (s, 2H), 4.55 (d, J = 5.8 Hz, 2H), 2.97 (s, 3H), 2.78 − 2.60 (m, 1H), 2.51 (s, 1H), 2.41 (s, 3H), 1.96-1.90 (m, 1H), 1.55-1.50 (m, 2H), 0.94 − 0.92 (m, 2H), 0.64-0.63 (m, 2H). |
| | rac-(1S*,2S*)-N-(6-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyrazin-2-yl)- | |
| | 2-(4-methylpyrimidin-2- | |
| | yl)cyclopropane-1-carboxamide | |
| | (I-526) | |
| Example 527 | 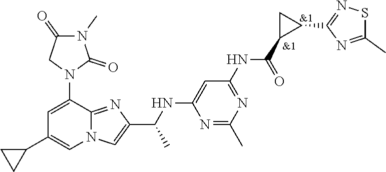 [SMILES: Cc1nc(NC(=O)[C@H]2C[C@@H]2c2nsc(C)n2)cc(N[C@H](C)c2cn3cc(C4CC4)cc(N4CC(=O)N(C)C4=O)c3n2)n1] | rac-(1S*,2S*)-N-(6-chloro-2-methylpyrimidin- 4-yl)-2-(5-methyl-1,2,4-thiadiazol-3- yl)cyclopropane-1-carboxamide ESI-MS: [M + H]+, 587.2, ¹H NMR (400 MHz, DMSO) δ 10.69 (s, 1H), 8.24 (s, 1H), 7.66 (d, J = 2.6 Hz, 2H), 7.34 (s, 1H), 7.12 (s, 1H), 5.40 (s, 1H), 4.95 − 4.94 (m, 2H), 2.97 (d, J = 0.8 Hz, 3H), 2.75 (s, 3H), 2.64 − 2.61 (m, 2H), 2.25 (s, 3H), 1.95 − 1.90 (m, 1H), 1.49 (d, J = 6.7 Hz, 5H), 0.96 − 0.91 (m, 2H), 0.65 − 0.61 (m, 2H). |
| | rac-(1S*,2S*)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(5- | |
| | methyl-1,2,4-thiadiazol-3- | |
| | yl)cyclopropane-1-carboxamide | |
| | (I-527) | |
| Example 528 | 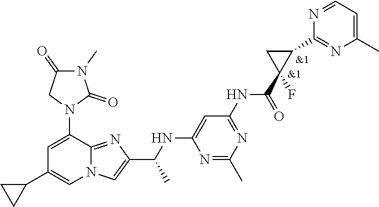 [SMILES: Cc1ccnc([C@H]2C[C@]2(F)C(=O)Nc2cc(N[C@H](C)c3cn4cc(C5CC5)cc(N5CC(=O)N(C)C5=O)c4n3)nc(C)n2)n1] | rac-(1S*,2S*)-N-(6-chloro-2-methylpyrimidin- 4-yl)-1-fluoro-2-(4-methylpyrimidin-2- yl)cyclopropane-1-carboxamide ESI-MS [M + H]⁺: 599.1, H NMR (400 MHz, DMSO) δ 10.06 (s, 1H), 8.62 (d, J = 5.0 Hz, 1H), 8.24 (s, 1H), 7.79 (s, 1H), 7.68 (s, 1H), 7.34 (s, 1H), 7.31 (d, J = 5.1 Hz, 1H), 7.13 (d, J = 14.1 Hz, 1H), 5.45 (s, 1H), 4.95 (s, 2H), 3.09 − 3.01 (m, 1H), 2.97 (s, 3H), 2.46 (s, 3H), 2.28 (d, J = 3.1 Hz, 3H), 2.04 − 1.81 (m, 1H), 1.54- 1.50 (m, 2H), 1.23 (s, 3H), 1.01 − 0.88 (m, 2H), 0.67-0.61 (m, 2H). |
| | rac-(1S*,2S*)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)-2- | |
| | methylpyrimidin-4-yl)-1-fluoro- | |
| | 2-(4-methylpyrimidin-2- | |
| | yl)cyclopropane-1-carboxamide | |
| | (I-528) | |
| Example 529 | 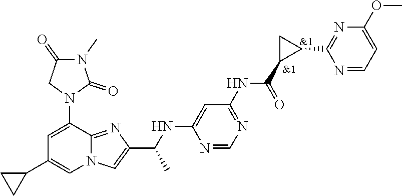 [SMILES: COc1ccnc([C@H]2C[C@@H]2C(=O)Nc2cc(N[C@H](C)c3cn4cc(C5CC5)cc(N5CC(=O)N(C)C5=O)c4n3)ncn2)n1] | rac-(1R,2R)-N-(6-chloropyrimidin-4-yl)-2-(4- methoxypyrimidin-2-yl)cyclopropane-1- carboxamide ESI-MS [M + H]+: 583.2, ¹H NMR (400 MHz, DMSO) δ 10.68 (s, 1H), 8.38 (d, J=5.8, 1H), 8.23 (s, 1H), 8.16 (s, 1H), 7.78 (s, 1H), 7.65 (d, J = 2.1, 1H), 7.34 (s, 1H), 7.30 (s, 1H), 6.75 (d, J = 5.8, 1H), 5.35 (s, 1H), 4.94 (s, 2H), 3.90 (s, 3H), 2.98 (d, J = 1.0, 3H), 2.72-2.68 (m, 1H), 2.54-2.52 (m, 1H), 1.94 − 1.92 (m, 1H), 1.55- 1.50 (m, 5H), 0.96-0.91 (m, 2H), 0.65-0.63 (m, 2H). |
| | rac-(1S*,2S*)-N-(6-(((R)-1-(6- | |
| | cyclopropyl-8-(3-methyl-2,4- | |
| | dioxoimidazolidin-1- | |
| | yl)imidazo[1,2-a]pyridin-2- | |
| | yl)ethyl)amino)pyrimidin-4-yl)- | |
| | 2-(4-methoxypyrimidin-2- | |
| | yl)cyclopropane-1-carboxamide | |
| | (I-529) | |
| Example 530 | 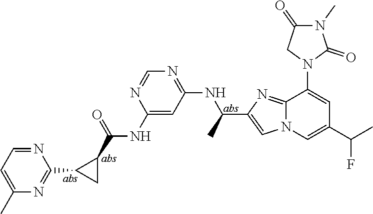 [SMILES: Cc1ccnc([C@H]2C[C@@H]2C(=O)Nc2cc(N[C@H](C)c3cn4cc(C(C)F)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H]+: 573.3. ¹H NMR (400 MHz, DMSO) δ 10.66 (s, 1H), 8.53 − 8.51 (m, 2H), 8.16 (s, 1H), 7.80 (s, 2H), 7.66 (s, 1H), 7.32 (s, 1H), 7.21 (d, J = 5.1 Hz, 1H), 5.83 − 5.66 (m, 1H), 5.39 (s, 1H), 5.03 − 4.93 (m, 2H), 2.98 (s, 3H), 2.63 − 2.60 (m, 1H), 2.6 − 2.54 (m, 1H), 2.41 (s, 3H), 1.65 − 1.57 (m, 3H), 1.52 (d, J = 6.7 Hz, 5H). |
| | (1S,2S)-N-(6-(((1R)-1-(6-(1- | |
| | fluoroethyl)-8-(3-methyl-2,4- | |
| | dioxoimidazolidin-1- | |
| | yl)imidazo[1,2-a]pyridin-2- | |
| | yl)ethyl)amino)pyrimidin-4-yl)- | |
| | 2-(4-methylpyrimidin-2- | |
| | yl)cyclopropane-1-carboxamide | |
| | (I-530) | |
| Example 531 | 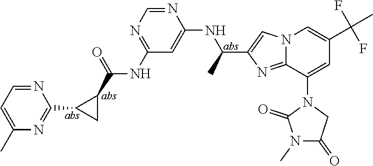 [SMILES: Cc1ccnc([C@H]2C[C@@H]2C(=O)Nc2cc(N[C@H](C)c3cn4cc(C(C)(F)F)cc(N5CC(=O)N(C)C5=O)c4n3)ncn2)n1] (1S,2S)-N-(6-(((R)-1-(6-(1,1- | (1S,2S)-N-(6-chloropyrimidin-4-yl)-2-(4- methylpyrimidin-2-yl)cyclopropane-1- carboxamide ESI-MS [M + H]+: 591.3, ¹H NMR (400 MHz, DMSO) δ 10.67 (s, 1H), 8.76 (d, J = 1.5 Hz, 1H), 8.52 (d, J = 5.1 Hz, 1H), 8.16 (d, J = 0.7 Hz, 1H), 7.89 − 7.80 (m, 2H), 7.75 (d, J = 1.5 Hz, 1H), 7.32 (s, 1H), 7.21 (d, J = 5.2 Hz, 1H), 5.37 (s, 1H), 4.99 (d, J = 1.5 Hz, 2H), 2.98 (s, 3H), 2.64 − 2.60 (m, 1H), 2.55 − 2.52 (m, 1H), 2.41 (s, 3H), 2.01 (t, J = 18.8 Hz, 3H), 1.54 − 1.50 (m, 5H). |
| | difluoroethyl)-8-(3-methyl-2,4- | |
| | dioxoimidazolidin-1- | |
| | yl)imidazo[1,2-a]pyridin-2- | |
| | yl)ethyl)amino)pyrimidin-4-yl)- | |
| | 2-(4-methylpyrimidin-2- | |
| | yl)cyclopropane-1-carboxamide | |
| | (I-531) | |
| Example 532 | 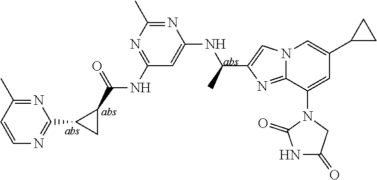 [SMILES: Cc1ccnc([C@H]2C[C@@H]2C(=O)Nc2cc(N[C@H](C)c3cn4cc(C5CC5)cc(N5CC(=O)NC5=O)c4n3)nc(C)n2)n1] (1S,2S)-N-(6-(((R)-1-(6- | (1S,2S)-N-(6-chloro-2-methyl-pyrimidin-4-yl)- 2-(4-methylpyrimidin-2-yl)cyclopropane-1- carboxamide ESI-MS [M + H]⁺: 567.2. ¹H NMR (400 MHz, DMSO) δ 11.34 (s, 1H), 10.65 (s, 1H), 8.52 (d, J = 5.1 Hz, 1H), 8.22 (d, J = 1.1 Hz, 1H), 7.67 − 7.64 (m, 2H), 7.32 (d, J = 1.4 Hz, 1H), 7.20 (d, J = 5.1 Hz, 1H), 7.12 (s, 1H), 5.38 (s, 1H), 4.91 (d, J = 17.9 Hz 2H), 2.55-2.52 (m, 2H), 2.41 (s, 3H), 2.24 (s, 3H), 1.95-1.89 (m, 1H), 1.51-1.48 (m, 5H), 0.95 − 0.91 (m, 2H), 0.65- 0.60 (m, 2H). |
| | cyclopropyl-8-(2,4- | |
| | dioxoimidazolidin-1- | |
| | yl)imidazo[1,2-a]pyridin-2- | |
| | yl)ethyl)amino)-2- | |
| | methylpyrimidin-4-yl)-2-(4- | |
| | methylpyrimidin-2- | |
| | yl)cyclopropane-1-carboxamide | |
| | (I-532) | |
| Example 533 | 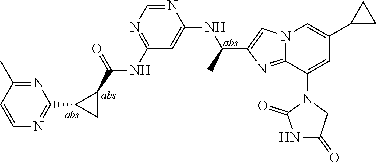 [SMILES: Cc1ccnc([C@H]2C[C@@H]2C(=O)Nc2cc(N[C@H](C)c3cn4cc(C5CC5)cc(N5CC(=O)NC5=O)c4n3)ncn2)n1] (1S,2S)-N-(6-(((R)-1-(6- cyclopropyl-8-(2,4- | (1S,2S)-N-(6-chloropyrimidin-4-yl)-2-(4- methylpyrimidin-2-yl)cyclopropane-1- carboxamide ESI-MS [M + H]+: 553.2. ¹H NMR (400 MHz, DMSO) δ 11.36 (s, 1H), 10.68 (s, 1H), 8.52 (d, J = 5.1 Hz, 1H), 8.17 (s, 1H), 8.16 (s, 1H), 7.83 (s, 1H), 7.66 (s, 1H), 7.33 (d, J = 15.5 Hz, 2H), 7.20 (d, J = 4.8 Hz, 1H), 5.36 (s, 1H), 4.89 (d, J = 2.9 Hz, 2H), 2.61-2.55 (m, 2H), 2.41 (s, 3H), 1.95-1.90 (m, 1H), 1.52(s, 3H), 1.24 − 1.20 (m, 2H), 0.97 − 0.90 (m, 2H), 0.66-0.60 (m, 2H). |
| | dioxoimidazolidin-1- | |
| | yl)imidazo[1,2-a]pyridin-2- | |
| | yl)ethyl)amino)pyrimidin-4-yl)- | |
| | 2-(4-methylpyrimidin-2- | |
| | yl)cyclopropane-1-carboxamide | |
| | (I-533) | |
| Example 534 | 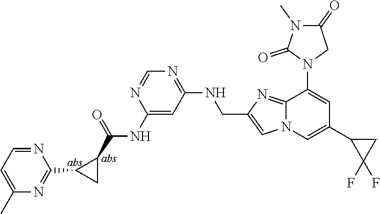 | (1S,2S)-N-(6-chloropyrimidin-4-yl)-2-(4- methylpyrimidin-2-yl)cyclopropane-1- carboxamide ESI-MS [M + H] +: 540.2, ¹H NMR (400 MHz, DMSO) δ 10.65 (s, 1H), 8.52 (d, J = 5.1 Hz, 1H), 8.23 (s, 1H), 8.16 (s, 1H), 7.77 (s, 1H), 7.64 (s, 1H), 7.30 (s, 1H), 7.23 − 7.19 (m, 2H), 5.33 (s, 1H), 4.54 − 4.44 (m, 4H), 2.63 − 2.60 (m, 1H), 2.56 − 2.54 (m, 1H), 2.42 (s, 3H), 1.96-1.90 (m, 1H), 1.58-1.45 (m, 5H), 0.95-0.90 (m, 2H), 0.64-0.63 (m, 2H). |
| | (1S,2S)-N-(6-(((R)-1-(6- | |
| | cyclopropyl-8-(2-oxooxazolidin- | |
| | 3-yl)imidazo[1,2-a]pyridin-2- | |
| | yl)ethyl)amino)pyrimidin-4-yl)- | |
| | 2-(4-methylpyrimidin-2- | |
| | yl)cyclopropane-1-carboxamide | |
| | (I-534) | |
| Example 535 | 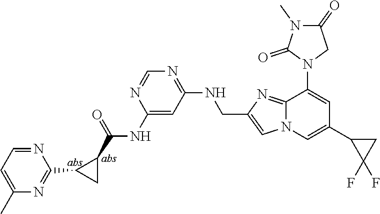 [SMILES: Cc1ccnc([C@H]2C[C@@H]2C(=O)Nc2cc(NCc3cn4cc(C5CC5(F)F)cc(N5CC(=O)N(C)C5=O)c4n3)ncn2)n1] | (1S,2S)-N-(6-chloropyrimidin-4-yl)-2-(4- methylpyrimidin-2-yl)cyclopropane-1- carboxamide ESI-MS [M + H] +: 589.2, ¹H NMR (400 MHz, DMSO) δ 10.68 (s, 1H), 8.52 (d, J = 5.1 Hz, 1H), 8.41 (s, 1H), 8.19 (s, 1H), 7.90 (s, 1H), 7.76 (s, 1H), 7.54 (s, 1H), 7.32 (s, 1H), 7.21 (d, J = 5.1 Hz, 1H), 4.93 (d, J = 18.5 Hz, 2H), 4.60 (s, 2H), 3.12 − 3.01 (m, 1H), 2.98 (s, 3H), 2.66 − 2.52 (m, 2H), 2.41 (s, 3H), 2.10 − 1.97 (m, 1H), 1.90 − 1.80 (m, 1H), 1.55 − 1.46 (m, 2H). |
| | (1S,2S)-N-(6-(((6-(2,2- | |
| | difluorocyclopropyl)-8-(3- | |
| | methyl-2,4-dioxoimidazolidin-1- | |
| | yl)imidazo[1,2-a]pyridin-2- | |
| | yl)methyl)amino)pyrimidin-4- | |
| | yl)-2-(4-methylpyrimidin-2- | |
| | yl)cyclopropane-1-carboxamide | |
| | (I-535) | |
| Example 536 | 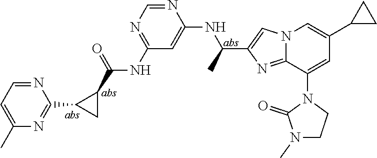 [SMILES: Cc1ccnc([C@H]2C[C@@H]2C(=O)Nc2cc(N[C@H](C)c3cn4cc(C5CC5)cc(N5CCN(C)C5=O)c4n3)ncn2)n1] (1S,2S)-N-(6-(((R)-1-(6- cyclopropyl-8-(3-methyl-2- | (1S,2S)-N-(6-chloropyrimidin-4-yl)-2-(4- methylpyrimidin-2-yl)cyclopropane-1- carboxamide ESI-MS [M + H] +: 553.7, ¹H NMR (400 MHz, DMSO) δ 10.65 (s, 1H), 8.52 (d, J = 5.1 Hz, 1H), 8.16 (d, J = 0.8 Hz, 1H), 8.10 (d, J = 1.1 Hz, 1H), 7.75 (s, 1H), 7.59 (s, 1H), 7.30 (s, 1H), 7.22 − 7.20 (m, 2H), 5.31 (s, 1H), 4.32 − 4.28 (m, 2H), 3.49 − 3.45 (m, 2H), 2.79 (s, 3H), 2.64 − 2.60 (m, 1H), 2.53 − 2.52 (m, 1H), 2.41 (s, 3H), 1.90 − 1.84 ( m, 1H), 1.55 − 1.49 (m, 5H), 0.92 − 0.87 (m, 2H), 0.62 − 0.59 (m, 2H). |
| | oxoimidazolidin-1- | |
| | yl)imidazo[1,2-a]pyridin-2- | |
| | yl)ethyl)amino)pyrimidin-4-yl)- | |
| | 2-(4-methylpyrimidin-2- | |
| | yl)cyclopropane-1-carboxamide | |
| | (I-536) | |
| Example 537 | 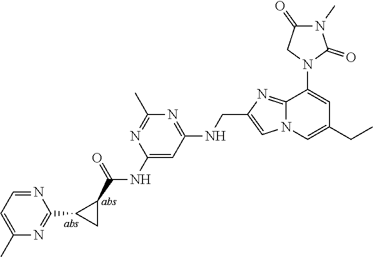 [SMILES: CCc1cc(N2CC(=O)N(C)C2=O)c2nc(CNc3cc(NC(=O)[C@H]4C[C@@H]4c4nccc(C)n4)nc(C)n3)cn2c1] | (1S,2S)-N-(6-chloro-2-methyl-pyrimidin-4-yl)- 2-(4-methylpyrimidin-2-yl)cyclopropane-1- carboxamide ESI-MS [M + H]+: 555.3 ¹H NMR (400 MHz, DMSO) δ 10.68 (s, 1H), 8.52 (d, J = 5.1 Hz, 1H), 8.26 (s, 1H), 7.74 (s, 2H), 7.47 (s, 1H), 7.20 (d, J = 5.1 Hz, 1H), 7.14 (s, 1H), 4.93 (s, 2H), 4.58 (s, 2H), 2.98 (s, 3H), 2.60-2.56 (m, J = 11.1 Hz, 4H), 2.41 (s, 3H), 2.27 (s, 3H), 1.54 − 1.426(m, 2H), 1.20 (t, J = 7.5 Hz, 3H). |
| | (1S,2S)-N-(6-(((6-ethyl-8-(3- | |
| | methyl-2,4-dioxoimidazolidin-1- | |
| | yl)imidazo[1,2-a]pyridin-2- | |
| | yl)methyl)amino)-2- | |
| | methylpyrimidin-4-yl)-2-(4- | |
| | methylpyrimidin-2- | |
| | yl)cyclopropane-1-carboxamide | |
| | (I-537) | |
| Example 538 | 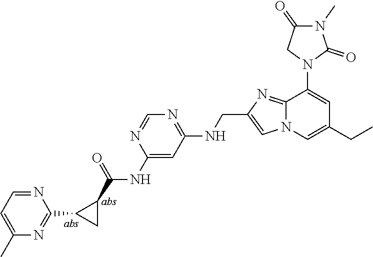 [SMILES: CCc1cc(N2CC(=O)N(C)C2=O)c2nc(CNc3cc(NC(=O)[C@H]4C[C@@H]4c4nccc(C)n4)ncn3)cn2c1] | (1S,2S)-N-(6-chloropyrimidin-4-yl)-2-(4- methylpyrimidin-2-yl)cyclopropane-1- carboxamide ESI-MS [M + H]+: 541.3, ¹H NMR (400 MHz, DMSO) δ 10.67 (s, 1H), 8.52 (d, J = 5.1 Hz, 1H), 8.25 (d, J = 1.0 Hz, 1H), 8.18 (s, 1H), 7.88 (s, 1H), 7.73 (s, 1H), 7.47 (d, J = 1.1 Hz, 1H), 7.31 (d, J = 0.7 Hz, 1H), 7.20 (d, J = 5.1 Hz, 1H), 4.93 (s, 2H), 4.58 (s, 2H), 2.98 (s, 3H), 2.64 − 2.56 (m, 3H), 2.55 − 2.52 (m, 1H), 2.41 (s, 3H), 1.55 − 1.47 (m, 2H), 1.20 (t, J = 7.5 Hz, 3H). |
| | (1S,2S)-N-(6-(((6-ethyl-8-(3- | |
| | methyl-2,4-dioxoimidazolidin-1- | |
| | yl)imidazo[1,2-a]pyridin-2-yl) | |
| | methyl)amino)pyrimidin-4-yl)- | |
| | 2-(4-methylpyrimidin-2-yl)cyclo | |
| | propane-1-carboxamide (I-538) | |

### Synthesis of rac-(1S*,2S*)—N-(4-cyclopropyl-6-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide (I-539)

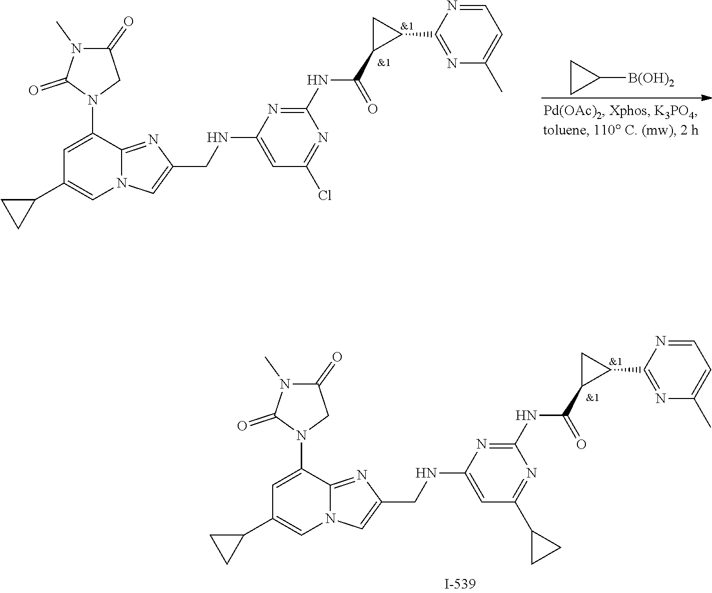 [SMILES: Cc1ccnc([C@H]2C[C@@H]2C(=O)Nc2nc(Cl)cc(NCc3cn4cc(C5CC5)cc(N5CC(=O)N(C)C5=O)c4n3)n2)n1 ; OB(O)C1CC1 ; Cc1ccnc([C@H]2C[C@@H]2C(=O)Nc2nc(NCc3cn4cc(C5CC5)cc(N5CC(=O)N(C)C5=O)c4n3)cc(C3CC3)n2)n1]

To a mixture of N-(4-chloro-6-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide (120 mg, 0.2 mmol), cyclopropylboronic acid (51.6 mg, 0.6 mmol), and Cs₂CO₃ (195.5 mg, 0.6 mmol) in dioxane (5 mL) were added Pd(OAc)₂ (8.9 g, 0.04 mmol) and Xphos (28.5 mg, 0.06 mmol). After degassing with N₂ for 1 min, the reaction was irradiated in microwave at 110° C. for 2 h. The reaction mixture was filtered through Celite and the filter cake was washed with DCM/MeOH (10/1, 20 mL). The filtrate was concentrated in vacuo to give the crude, which was purified by preparative TLC (eluent: DCM/MeOH=20/1) to give N-(4-cyclopropyl-6-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide (3.3 mg, 2.8%) as a white solid. ESI-MS [M+H]+: 593.2, ¹H NMR (400 MHz, DMSO) δ 9.95 (s, 1H), 8.47 (d, J=4.9 Hz, 1H), 8.23 (s, 1H), 7.74 (s, 1H), 7.67 (s, 1H), 7.32 (s, 1H), 7.15 (d, J=5.0 Hz, 1H), 6.16 (s, 1H), 4.89 (s, 2H), 4.50 (s, 2H), 2.97 (s, 3H), 2.54-2.52 (m, 2H), 2.38 (s, 3H), 2.01-1.98 (m, 1H), 1.77-1.71 (m, 1H), 1.58-1.45 (m, 2H), 0.96-0.90 (m, 2H), 0.89-0.72 (m, 4H), 0.66-0.63 (m, 2H).

### Example 540

### Synthesis of rac-(1S*,2S*)—N-(4-(((6-cyclopropyl-8-(3-(2-hydroxyethyl)-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide (I-540)

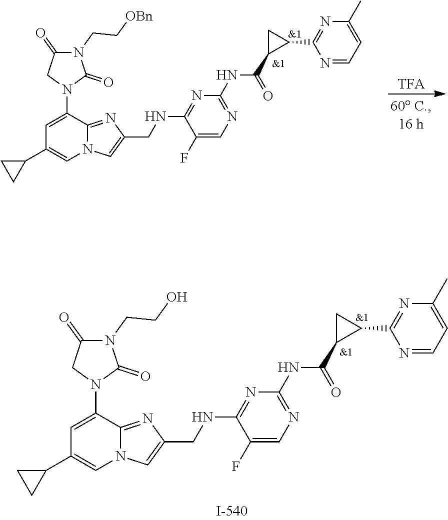 [SMILES: Cc1ccnc([C@H]2C[C@@H]2C(=O)Nc2ncc(F)c(NCc3cn4cc(C5CC5)cc(N5CC(=O)N(CCOCc6ccccc6)C5=O)c4n3)n2)n1 ; Cc1ccnc([C@H]2C[C@@H]2C(=O)Nc2ncc(F)c(NCc3cn4cc(C5CC5)cc(N5CC(=O)N(CCO)C5=O)c4n3)n2)n1]

A solution of rac-(1S*,2S*)—N-(4-(((8-(3-(2-(benzyloxy)ethyl)-2,4-dioxoimidazolidin-1-yl)-6-cyclopropylimidazo[1,2-a]pyridin-2-yl)methyl)amino)-5-fluoropyrimidin-2-yl)-2-(4-methylpyrimidin-2-yl)cyclopropane-1-carboxamide (50 mg, 0.072 mmol) in TFA (2 mL) was stirred at 60° C. for 16 h. After cooling to room temperature, the mixture was basified to pH˜8 with a solution of saturated aq·NaHCO₃ (15 mL) and then extracted with DCM (2×20 mL). The combined organic layers were washed with brine (30 mL), dried over anhydrous Na₂SO₄, and concentrated in vacuo to give the crude, which was purified by preparative TLC (eluent: DCM/MeOH=10:1) to give rac-(1S*,2S*)—N-(4-(((6-cyclopropyl-8-(3-(2-hydroxyethyl)-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide (27 mg, 62%) as a yellow solid. ESI-MS [M+H]+: 601.2. ¹H NMR (400 MHz, DMSO) δ 10.42 (s, 1H), 8.47 (d, J=4.6 Hz, 1H), 8.30-8.15 (m, 2H), 8.01 (s, 1H), 7.75 (d, J=34.0 Hz, 1H), 7.32 (s, 1H), 7.15 (s, 1H), 4.97-4.85 (m, 3H), 4.62 (s, 2H), 3.61-3.52 (m, 4H), 2.87-2.86 (m, 1H), 2.58-2.56 (m, 1H), 2.41 (s, 3H), 2.03-1.88 (m, 1H), 1.57-1.40 (m, 2H), 0.99-0.89 (m, 2H), 0.69-0.58 (m, 2H).

### Example 541

### Synthesis of rac-(1S*,2S*)—N-(4-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-2-yl)-2-(4-methylpyrimidin-2-yl)cyclopropane-1-carboxamide (I-541)

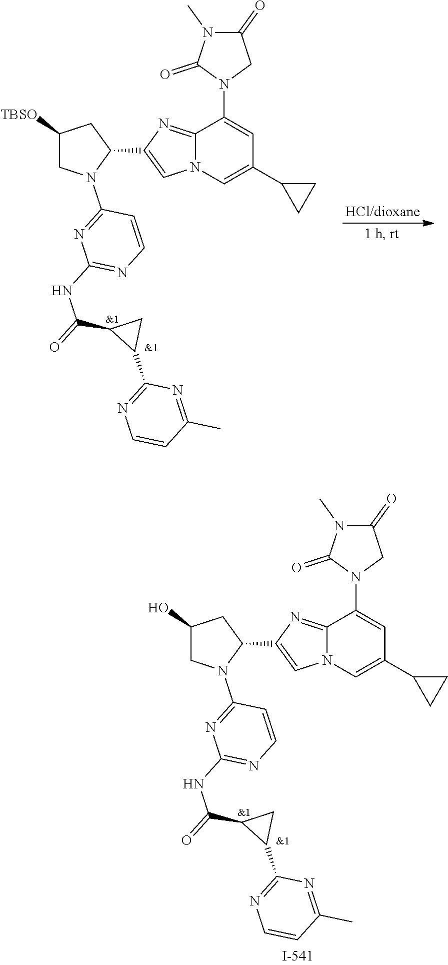

To a solution of rac-(1S*,2S*)—N-(4-((2R,4S)-4-((tert-butyldimethylsilyl)oxy)-2-(6-cyclopropyl-8-(3-methyl-2,4-dioxoimidazolidin-1-yl)imidazo[1,2-a]pyridin-2-yl)pyrrolidin-1-yl)pyrimidin-2-yl)-2-(4-methylpyrimidin-2-yl)cyclopropane-1-carboxamide (45 mg, 0.06 mmol) in MeOH (5 mL) was added HCl (4 M solution in dioxane, 1 mL). The resulting mixture was stirred at room temperature for 1 h, then ammonia in methanol (2 mL, 7M) was added. The reaction mixture was concentrated in vacuo and purified by preparative TLC to give rac-(1S*,2S*)—N-(4-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-2-yl)-2-(4-methylpyrimidin-2-yl)cyclopropane-1-carboxamide (11.3 mg, 31%) as a yellow solid. ESI-MS [M+H]+: 609.2. ¹H NMR (400 MHz, DMSO) δ 10.19 (s, 1H), 8.50-8.48 (d, J=5.1 Hz, 1H), 8.31 (s, 2H), 8.18-8.14 (m, 1H), 7.73-7.72 (m, 1H), 7.32 (s, 1H), 7.17 (d, J=5.0 Hz, 1H), 5.08-5.01 (m, 1H), 4.93-4.87 (m, 2H), 4.83-4.79 (m, 1H), 4.47-4.42 (m, 1H), 3.67-3.63 (m, 2H), 2.93 (d, J=1.7 Hz, 3H), 2.65-2.63 (m, 2H), 2.39 (m, 3H), 2.30-2.29 (m, 2H), 1.92-1.88 (m, 1H), 1.55-1.47 (m, 2H), 0.91-0.89 (m, 2H), 0.60-0.55 (m, 2H).

The compound in Table 39 was prepared in a similar manner to rac-(1S*,2S*)—N-(4-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-2-yl)-2-(4-methylpyrimidin-2-yl)cyclopropane-1-carboxamide from the corresponding silyl ether.

TABLE 39

| Example | Structure | Analytical Data |
|---|---|---|
| Example 542 | 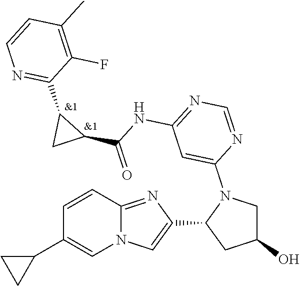 [SMILES: Cc1ccnc([C@H]2C[C@@H]2C(=O)Nc2cc(N3C[C@@H](O)C[C@@H]3c3cn4cc(C5CC5)ccc4n3)ncn2)c1F] | ESI-MS [M + H]⁺: 514.2, ¹H NMR (400 MHz, DMSO) δ 10.77-10.63 (m, 1H), 8.24-8.15 (m, 3H), 7.62-7.51 (m, 1H), 7.38 (d, J = 8.9 Hz, 1H), 7.18-7.17 (m, 2H), 6.96 (d, J = 8.8 Hz, 1H), 5.46-4.99 (m, 2H), 4.57-4.44 (m, 1H), 3.76 (s, 1.5H), 3.40 − 3.38 (m, 1H), 3.20 (s, 0.5H), 2.75 − 2.61 (m, 1H), 2.31 − 2.23 (m, 4H), 1.96 − 1.85 (m, 1H), 1.61 − 1.40 (m, 2H), 0.94 − 0.87 (m, 2H), 0.70 − 0.60 (m, 2H). |
| | rac-(1S*,2S*)-N-(6-((2R,4S)-2-(6- | |
| | cyclopropylimidazo[1,2-a]pyridin-2- | |
| | yl)-4-hydroxypyrrolidin-1- | |
| | yl)pyrimidin-4-yl)-2-(3-fluoro-4- | |
| | methylpyridin-2-yl)cyclopropane-1- | |
| | carboxamide (I-542) | |

### Examples 543-545

### Synthesis of rac-(1S*,3S*)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide (I-543)

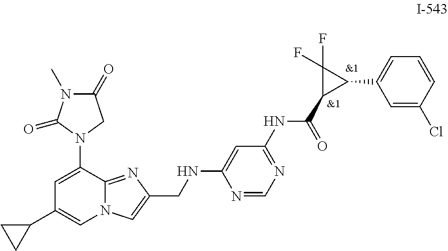 [SMILES: CN1C(=O)CN(c2cc(C3CC3)cn3cc(CNc4cc(NC(=O)[C@@H]5[C@@H](c6cccc(Cl)c6)C5(F)F)ncn4)nc23)C1=O]

The title compound was prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide as a mixture of enantiomers. ESI-MS [M+H]+: 607.2, ¹H NMR (400 MHz, DMSO) δ 10.80 (s, 1H), 8.24 (s, 2H), 7.98 (s, 1H), 7.70 (s, 1H), 7.48-7.39 (m, 3H), 7.37-7.26 (m, 3H), 4.91 (s, 2H), 4.60 (s, 2H), 3.72-3.63 (m, 1H), 3.55-3.47 (m, 1H), 2.96 (s, 3H), 1.99-1.88 (m, 1H), 0.97-0.90 (m, 2H), 0.67-0.60 (m, 2H).

The mixture was separated using SFC (Daicel CHIRALPAK OJ-H 250 mm×20 mm I.D., 5 m, CO₂/EtOH=54/46, 50 g/min, 35° C.) to give two enantiomers: (1S,3S)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide and (1R,3R)-3-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluorocyclopropane-1-carboxamide.

First eluting isomer (I-544): ESI-MS [M+H]+: 607.2, ¹H NMR (400 MHz, DMSO) δ 10.80 (s, 1H), 8.24 (s, 2H), 7.98 (s, 1H), 7.70 (s, 1H), 7.47-7.39 (m, 3H), 7.36-7.26 (m, 3H), 4.91 (s, 2H), 4.60 (s, 2H), 3.72-3.62 (m, 1H), 3.55-3.46 (m, 1H), 2.96 (s, 3H), 1.96-1.90 (m, 1H), 0.97-0.89 (m, 2H), 0.67-0.60 (m, 2H). RT=4.314 min, 100% e.e.

Second eluting isomer (I-545): ESI-MS [M+H]+: 607.2, ¹H NMR (400 MHz, DMSO) δ 10.80 (s, 1H), 8.24 (s, 2H), 7.98 (s, 1H), 7.70 (s, 1H), 7.48-7.39 (m, 3H), 7.37-7.26 (m, 3H), 4.91 (s, 2H), 4.60 (s, 2H), 3.72-3.63 (m, 1H), 3.55-3.47 (m, 1H), 2.96 (s, 3H), 1.99-1.88 (m, 1H), 0.97-0.90 (m, 2H), 0.67-0.60 (m, 2H). RT=7.144 min, 100% e.e.

### Examples 546-548

### Synthesis of rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide (I-546)

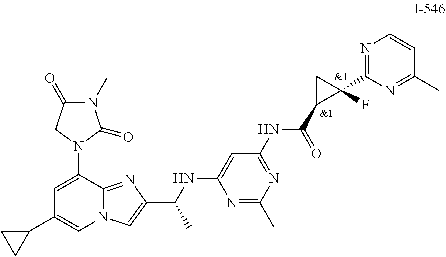 [SMILES: Cc1ccnc([C@@]2(F)C[C@@H]2C(=O)Nc2cc(N[C@H](C)c3cn4cc(C5CC5)cc(N5CC(=O)N(C)C5=O)c4n3)nc(C)n2)n1]

The title compound was prepared in a similar manner to rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide as a mixture of diastereomers. ESI-MS (M+H)+: 599.3, ¹H NMR (400 MHz, DMSO) δ 10.64 (s, 1H), 8.66 (d, J=5.0 Hz, 1H), 8.24 (s, 1H), 7.66 (d, J=7.2 Hz, 2H), 7.34 (d, J=4.3 Hz, 2H), 7.15-7.06 (m, 1H), 5.41-5.32 (m, 1H), 4.95 (s, 2H), 2.98 (s, 3H), 2.93-2.82 (m, 1H), 2.49 (s, 3H), 2.23 (s, 3H), 1.94-1.86 (m, 2H), 1.50 (d, J=6.7 Hz, 3H), 1.00-0.88 (m, 2H), 0.72-0.57 (m, 2H).

The mixture was separated using SFC 80 (Daicel CHIRALPAK OJ-H 20×250 mm, 5.0 μm, CO2/MeOH (0.2% NH₃ (7M in MeOH))=75/25, 60 g/min, 35° C.) to give two diastereomers: (1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide and (1R,2R)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide.

First eluting isomer (I-547): ESI-MS [M+H]+: 599.3, ¹H NMR (400 MHz, DMSO) δ 10.64 (s, 1H), 8.66 (d, J=5.1 Hz, 1H), 8.24 (d, J=1.0 Hz, 1H), 7.68-7.64 (m, 2H), 7.34 (d, J=5.2 Hz, 2H), 7.13 (s, 1H), 5.42 (s, 1H), 4.95 (s, 2H), 2.97 (s, 3H), 2.93-2.82 (m, 1H), 2.49 (s, 3H), 2.26-2.13 (m, 4H), 1.95-1.87 (m, 2H), 1.50 (d, J=6.7 Hz, 3H), 0.98-0.90 (m, 2H), 0.66-0.60 (m, 2H). RT=2.52 min, 100.0% e.e.

Second eluting isomer (I-548): ESI-MS [M+H]+: 599.3, ¹H NMR (400 MHz, DMSO) δ 10.65 (s, 1H), 8.66 (d, J=5.1 Hz, 1H), 8.25 (s, 1H), 7.68 (s, 2H), 7.34 (d, J=5.1 Hz, 2H), 7.13 (s, 1H), 5.37 (s, 1H), 4.95 (s, 2H), 2.98 (s, 3H), 2.91-2.87 (m, 1H), 2.48 (s, 3H), 2.27-2.15 (m, 4H), 1.93-1.87 (m, 2H), 1.50 (d, J=6.7 Hz, 3H), 0.99-0.89 (m, 2H), 0.66-0.61 (m, 2H). RT=4.12 min, 100.0% e.e.

### Synthesis of 1-(2-(1-((6-chloropyrimidin-4-yl)oxy)ethyl)-6-cyclopropylimidazo[1,2-a]pyridin-8-yl)-3-methylimidazolidine-2,4-dione

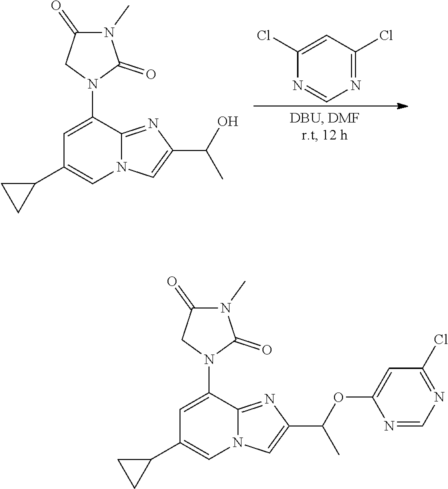 [SMILES: CC(O)c1cn2cc(C3CC3)cc(N3CC(=O)N(C)C3=O)c2n1 ; Clc1cc(Cl)ncn1 ; CC(Oc1cc(Cl)ncn1)c1cn2cc(C3CC3)cc(N3CC(=O)N(C)C3=O)c2n1]

To a solution of 1-(6-cyclopropyl-2-(1-hydroxyethyl)imidazo[1,2-a]pyridin-8-yl)-3-methylimidazolidine-2,4-dione (100 mg, 0.32 mmol) in DMF (3 mL) was added 4,6-dichloropyrimidine (284 mg, 1.92 mmol) and DBU (145 mg, 0.96 mmol), and then the mixture was stirred at 45° C. for 48 h. The reaction mixture was quenched by water (20 mL) and extracted with EtOAc (3×30 mL). The combined organic layers were concentrated to dryness. The residue was purified by preparative TLC (eluted with PB:EtOAc=3:1) to give 1-(2-(1-((6-chloropyrimidin-4-yl)oxy)ethyl)-6-cyclopropylimidazo[1,2-a]pyridin-8-yl)-3-methylimidazolidine-2,4-dione as a yellow oil (30 mg, yield 220M). ESI-MS [M+H]⁺: 427.1

The compounds in Table 40 were prepared in a similar manner to 1-(2-(1-((6-chloropyrimidin-4-yl)oxy)ethyl)-6-cyclopropylimidazo[1,2-a]pyridin-8-yl)-3-methylimidazolidine-2,4-dione from a di-halo-pyrimidine and appropriate alcohol.

TABLE 40

| Structure | Analytical Data |
|---|---|
| 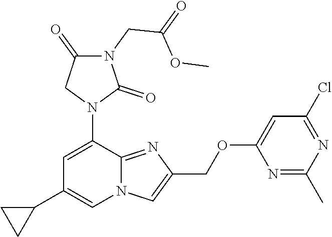 [SMILES: COC(=O)CN1C(=O)CN(c2cc(C3CC3)cn3cc(COc4cc(Cl)nc(C)n4)nc23)C1=O] | ESI-MS [M + H]⁺: 485.2 |
| methyl 2-(3-(2-(((6-chloro-2-methylpyrimidin-4- | |
| yl)oxy)methyl)-6-cyclopropylimidazo[1,2-a]pyridin-8- | |
| yl)-2,5-dioxoimidazolidin-1-yl)acetate | |
| 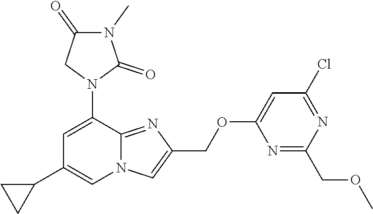 [SMILES: COCc1nc(Cl)cc(OCc2cn3cc(C4CC4)cc(N4CC(=O)N(C)C4=O)c3n2)n1] | ESI-MS [M + H]⁺: 457.2 |
| 1-(2-(((6-chloro-2-(methoxymethyl)pyrimidin-4- | |
| yl)oxy)methyl)-6-cyclopropylimidazo[1,2-a]pyridin-8- | |
| yl)-3-methylimidazolidine-2,4-dione | |
| 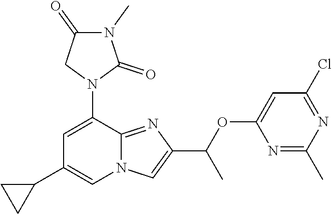 [SMILES: Cc1nc(Cl)cc(OC(C)c2cn3cc(C4CC4)cc(N4CC(=O)N(C)C4=O)c3n2)n1] | ESI-MS [M + H]⁺: 441.2 |
| 1-(2-(1-((6-chloro-2-methylpyrimidin-4-yl)oxy)ethyl)-6- | |
| cyclopropylimidazo[1,2-a]pyridin-8-yl)-3- | |
| methylimidazolidine-2,4-dione | |
| 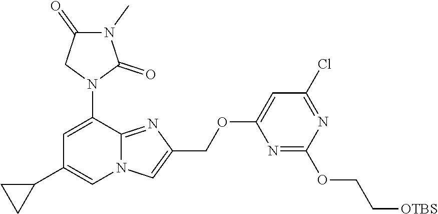 [SMILES: CN1C(=O)CN(c2cc(C3CC3)cn3cc(COc4cc(Cl)nc(OCCO[Si](C)(C)C(C)(C)C)n4)nc23)C1=O] | ESI-MS [M + H]⁺: 587.2 |
| 1-(2-(((2-(2-((tert-butyldimethylsilyl)oxy)ethoxy)-6- | |
| chloropyrimidin-4-yl)oxy)methyl)-6- | |
| cyclopropylimidazo[1,2-a]pyridin-8-yl)-3- | |
| methylimidazolidine-2,4-dione | |
| 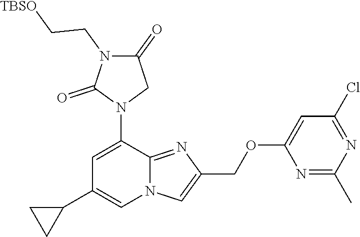 [SMILES: Cc1nc(Cl)cc(OCc2cn3cc(C4CC4)cc(N4CC(=O)N(CCO[Si](C)(C)C(C)(C)C)C4=O)c3n2)n1] | ESI-MS [M + H]⁺: 571.2 |
| 3-(2-((tert-butyldimethylsilyl)oxy)ethyl)-1-(2-(((6- | |
| chloro-2-methylpyrimidin-4-yl)oxy)methyl)-6- | |
| cyclopropylimidazo[1,2-a]pyridin-8-yl)imidazolidine- | |
| 2,4-dione | |
| 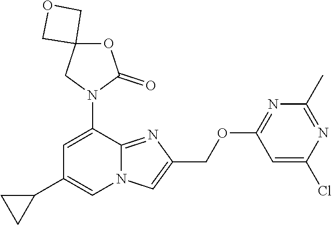 [SMILES: Cc1nc(Cl)cc(OCc2cn3cc(C4CC4)cc(N4CC5(COC5)OC4=O)c3n2)n1] | ESI-MS [M + H]⁺: 442.2 |
| 7-(2-(((6-chloro-2-methylpyrimidin-4-yl)oxy)methyl)-6- | |
| cyclopropylimidazo[1,2-a]pyridin-8-yl)-2,5-dioxa-7- | |
| azaspiro[3.4]octan-6-one | |
| 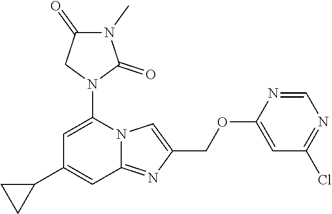 [SMILES: CN1C(=O)CN(c2cc(C3CC3)cc3nc(COc4cc(Cl)ncn4)cn23)C1=O] | ESI-MS [M + H]⁺: 413.2 |
| 1-(2-(((6-chloropyrimidin-4-yl)oxy)methyl)-7- | |
| cyclopropylimidazo[1,2-a]pyridin-5-yl)-3- | |
| methylimidazolidine-2,4-dione | |
| 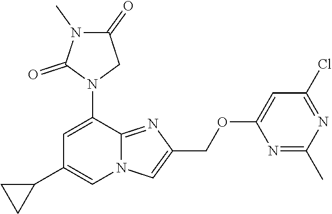 [SMILES: Cc1nc(Cl)cc(OCc2cn3cc(C4CC4)cc(N4CC(=O)N(C)C4=O)c3n2)n1] | ESI-MS [M + H]⁺: 427.2. ¹H NMR (400 MHz, DMSO) δ 8.29 (d, J = 1.2 Hz, 1H), 7.98 (s, 1H), 7.39 (d, J = 1.4 Hz, 1H), 7.04 (s, 1H), 5.51 (s, 2H), 4.88 (s, 2H), 2.97 (s, 3H), 2.55 (s, 3H), 2.00 − 1.93 (m, 1H), 0.98 − 0.94 (m, 2H), 0.68 − 0.64 (m, 2H). |
| 1-(2-(((6-chloro-2-methylpyrimidin-4-yl)oxy)methyl)-6- | |
| cyclopropylimidazo[1,2-a]pyridin-8-yl)-3- | |
| methylimidazolidine-2,4-dione | |
| 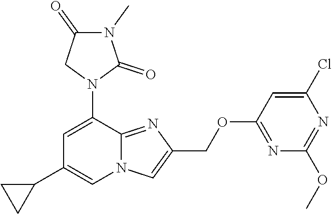 [SMILES: COc1nc(Cl)cc(OCc2cn3cc(C4CC4)cc(N4CC(=O)N(C)C4=O)c3n2)n1] | ESI-MS [M + H]⁺: 443.2 |
| 1-(2-(((6-chloro-2-methoxypyrimidin-4-yl)oxy)methyl)- | |
| 6-cyclopropylimidazo[1,2-a]pyridin-8-yl)-3- | |
| methylimidazolidine-2,4-dione | |
| 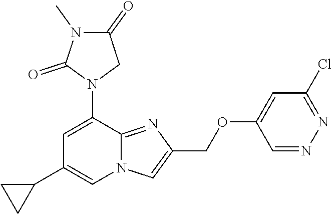 [SMILES: CN1C(=O)CN(c2cc(C3CC3)cn3cc(COc4cnnc(Cl)c4)nc23)C1=O] | ESI-MS (M + H)⁺: 413.3 |
| 1-(2-(((6-chloropyridazin-4-yl)oxy)methyl)-6- | |
| cyclopropylimidazo[1,2-a]pyridin-8-yl)-3- | |
| methylimidazolidine-2,4-dione | |
| 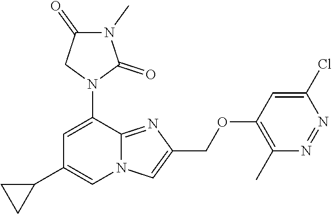 [SMILES: Cc1nnc(Cl)cc1OCc1cn2cc(C3CC3)cc(N3CC(=O)N(C)C3=O)c2n1] | ESI-MS (M + H)⁺: 427.2 |
| 1-(2-(((6-chloro-3-methylpyridazin-4-yl)oxy)methyl)-6- | |
| cyclopropylimidazo[1,2-a]pyridin-8-yl)-3- | |
| methylimidazolidine-2,4-dione | |
| 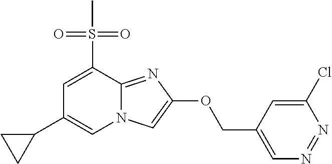 [SMILES: CS(=O)(=O)c1cc(C2CC2)cn2cc(OCc3cnnc(Cl)c3)nc12] | ESI-MS (M + H)⁺: 379.1 |
| 2-(((6-chloropyridazin-4-yl)oxy)methyl)-6-cyclopropyl- | |
| 8-(methylsulfonyl)imidazo[1,2-a]pyridine | |

### Example 549

### Synthesis of (1S,2S)—N-(6-(1-(6-cyclopropyl-8-(3-methyl-2,4-dioxoimidazolidin-1-yl)imidazo[1,2-a]pyridin-2-yl)ethoxy)pyrimidin-4-yl)-2-(4-methylpyrimidin-2-yl)cyclopropane-1-carboxamide (I-549)

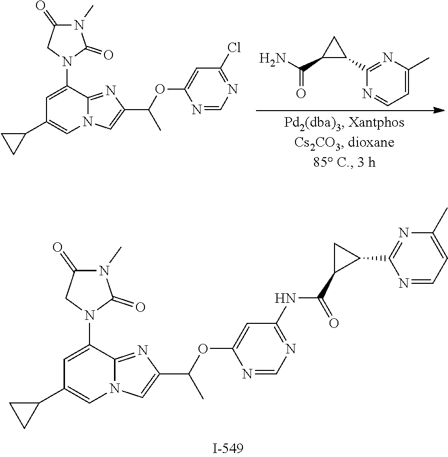 [SMILES: CC(Oc1cc(Cl)ncn1)c1cn2cc(C3CC3)cc(N3CC(=O)N(C)C3=O)c2n1 ; Cc1ccnc([C@H]2C[C@@H]2C(N)=O)n1 ; Cc1ccnc([C@H]2C[C@@H]2C(=O)Nc2cc(OC(C)c3cn4cc(C5CC5)cc(N5CC(=O)N(C)C5=O)c4n3)ncn2)n1]

To a solution of 1-(2-(1-((6-chloropyrimidin-4-yl)oxy)ethyl)-6-cyclopropylimidazo[1,2-a]pyridin-8-yl)-3-methylimidazolidine-2,4-dione (30 mg, 0.07 mmol) in dioxane (3 mL) was added (1S,2S)-2-(4-methylpyrimidin-2-yl)cyclopropane-1-carboxamide (12 mg, 0.07 mmol), Pd₂(dba)₃ (9 mg, 0.01 mmol), Xantphos (6 mg, 0.01 mmol), and Cs₂CO₃ (69 mg, 0.21 mmol). The mixture was degassed with N₂ three times and the mixture was stirred at 85° C. for 6 hr. After cooling to room temperature, the mixture was diluted with water (10 mL) and extracted with EtOAc (3×30). The combined organic layers were washed with brine, dried over anhydrous Na₂SO₄, filtered, and concentrated in vacuo. The residue was purified by preparative TLC (eluted with DCM:MeOH=20:1) to give (1S,2S)—N-(6-(1-(6-cyclopropyl-8-(3-methyl-2,4-dioxoimidazolidin-1-yl)imidazo[1,2-a]pyridin-2-yl)ethoxy)pyrimidin-4-yl)-2-(4-methylpyrimidin-2-yl)cyclopropane-1-carboxamide as white solid (12 mg, 30% ). ESI-MS (M+H)+: 568.2, ¹H NMR (400 MHz, DMSO) δ 11.17 (s, 1H), 8.52-8.49 (min, 2H), 8.26 (s, 1H), 7.85 (d, J=2.0 Hz, 1H), 7.48 (s, 1H), 7.40-7.35 (m, 1H), 7.22 (d, J=5.0 Hz, 1H), 6.39-6.32 (m, 1H), 4.91 (d, J=1.7 Hz, 2H), 2.97 (d, J=1.5 Hz, 3H), 2.68-2.64 (m, 1H), 2.58-2.55 (min, 1H), 2.44-52.39 (m, 3H), 2.00-1.91 (m, 1H), 1.68 (d, J=6.5 Hz, 3H), 1.60-1.51 (m, 2H), 0.98-20.91 (m, 2H), 0.67-0.61 (m, 2H).

The compounds in Table 41 were prepared in a similar manner to (1S,2S)—N-(6-(1-(6-cyclopropyl-8-(3-methyl-2,4-dioxoimidazolidin-1-yl)imidazo[1,2-a]pyridin-2-yl)ethoxy)pyrimidin-4-yl)-2-(4-methylpyrimidin-2-yl)cyclopropane-1-carboxamide from the appropriate cyclopropyl

TABLE 41

| | | Coupling Partner/ |
|---|---|---|
| Example | Structure | Analytical Data |
| Example 550 | 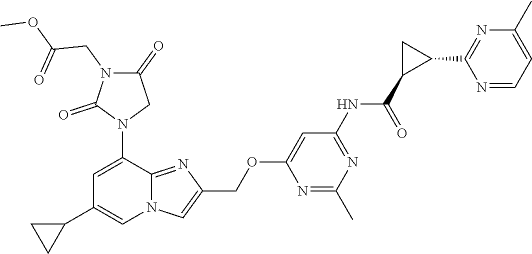 | methyl 2-(3-(2-(((6-chloro-2- methylpyrimidin-4-yl)oxy)methyl)-6- cyclopropylimidazo[1,2-a] pyridin-8-yl)-2,5- dioxoimidazolidin-1-yl)acetate ESI-MS (M + H)+: 626.3, ¹H NMR (400 MHz, MeOD) δ 8.46 (d, J = 5.2 Hz, 1H), 8.22 (d, J = 0.9 Hz, 1H), 7.89 (s, 1H), 7.41 (s, 1H), 7.36 (d, J = 1.5 Hz, 1H), 7.17 (d, J = 5.2 Hz, 1H), 5.51 (s, 2H), 4.82 (s, 2H), 4.40 (s, 2H), 3.79 (s, 3H), 2.73 − 2.68 (m, 1H), 2.56 − 2.52 (m, 1H), 2.47 (d, J = 1.5 Hz, 6H), 1.99 − 1.94 (m, 1H), 1.69 − 1.65 (m, 2H), 1.01 − 0.97 (m, 2H), 0.77 − 0.73 (m, 2H). |
| | methyl 2-(3-(6-cyclopropyl-2-(((2- | |
| | methyl-6-((1S,2S)-2-(4- | |
| | methylpyrimidin-2- | |
| | yl)cyclopropane-1- | |
| | carboxamido)pyrimidin-4- | |
| | yl)oxy)methyl)imidazo[1,2- | |
| | a]pyridin-8-yl)-2,5- | |
| | dioxoimidazolidin-1-yl)acetate (I-550) | |
| Example 551 | 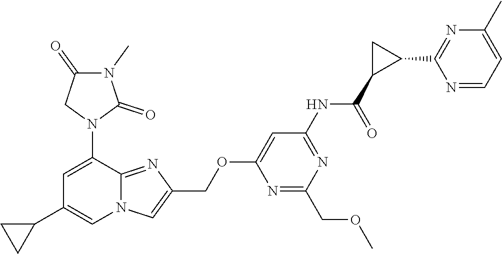 [SMILES: COCc1nc(NC(=O)[C@H]2C[C@@H]2c2nccc(C)n2)cc(OCc2cn3cc(C4CC4)cc(N4CC(=O)N(C)C4=O)c3n2)n1] | 1-(2-(((6-chloro-2- (methoxymethyl)pyrimidin-4- yl)oxy)methyl)-6-cyclopropylimidazo[1,2- a]pyridin-8-yl)-3-methylimidazolidine-2,4- dione ESI-MS (M + H)+: 598.1, ¹H NMR (400 MHz, DMSO) δ 11.31 (s, 1H), 8.53 (d, J = 5.1 Hz, 1H), 8.28 (s, 1H), 7.96 (s, 1H), 7.39 (d, J = 4.3 Hz, 2H), 7.21 (d, J = 5.1 Hz, 1H), 5.48 (s, 2H), 4.89 (s, 2H), 4.43 (s, 2H), 3.37 (s, 3H), 2.97 (s, 3H), 2.69-2.65(m, 1H), 2.54-2.52 (m, 1H), 2.41 (s, 3H), 1.99-1.95 (m, 1H), 1.56 − 1.53(m, 2H), 0.97-0.93 (m, 2H), 0.68-0.65 (m, 2H). |
| | (1S,2S)-N-(6-((6-cyclopropyl-8-(3- | |
| | methyl-2,4-dioxoimidazolidin-1- | |
| | yl)imidazo[1,2-a]pyridin-2- | |
| | yl)methoxy)-2- | |
| | (methoxymethyl)pyrimidin-4-yl)-2- | |
| | (4-methylpyrimidin-2- | |
| | yl)cyclopropane-1-carboxamide (I- | |
| | 551) | |
| Example 552 | 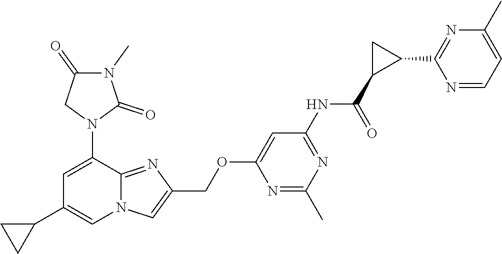 | 1-(2-(1-((6-chloro-2-methylpyrimidin-4- yl)oxy)ethyl)-6-cyclopropylimidazo[1,2- a]pyridin-8-yl)-3-methylimidazolidine-2,4- dione ESI-MS (M + H)+: 582.2, ¹H NMR (400 MHz, DMSO) δ 11.14 (s, 1H), 8.53 (d, J = 5.1 Hz, 1H), 8.23 (s, 1H), 7.86 (d, J = 2.2 Hz, 1H), 7.38 (s, 1H), 7.29 (s, 1H), 7.21 (d, J = 5.0 Hz, 1H), 6.38 (d, J = 6.6 Hz, 1H), 4.92 (s, 2H), 2.97 (d, J = 1.2 Hz, 3H), 2.66 − 2.59 (m, 1H), 2.56-2.54 (m, 1H), 2.43 (s, 3H), 2.41 (s, 3H), 2.02 − 1.89 (m, 1H), 1.67 (d, J = 6.5 Hz, 3H), 1.58-1.50 (m, 2H), 0.97-0.94 (m, 2H), 0.66-0.63 (m, 2H). |
| | (1S,2S)-N-(6-(1-(6-cyclopropyl-8- | |
| | (3-methyl-2,4-dioxoimidazolidin-1- | |
| | yl)imidazo[1,2-a]pyridin-2- | |
| | yl)ethoxy)-2-methylpyrimidin-4- | |
| | yl)-2-(4-methylpyrimidin-2- | |
| | yl)cyclopropane-1-carboxamide (I- | |
| | 552) | |
| Example 553 | 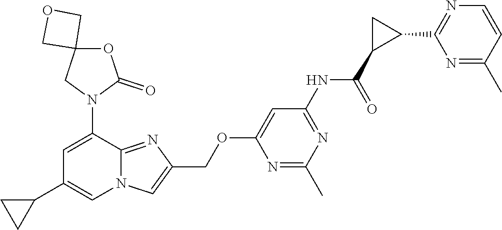 [SMILES: Cc1ccnc([C@H]2C[C@@H]2C(=O)Nc2cc(OCc3cn4cc(C5CC5)cc(N5CC6(COC6)OC5=O)c4n3)nc(C)n2)n1] | 7-(2-(((6-chloro-2-methylpyrimidin-4- yl)oxy)methyl)-6-cyclopropylimidazo[1,2- a]pyridin-8-yl)-2,5-dioxa-7- azaspiro[3.4]octan-6-one ESI-MS (M + H)+: 583.2, ¹H NMR (400 MHz, DMSO) δ 11.18 (s, 1H), 8.53 (d, J = 5.1 Hz, 1H), 8.28 (d, J = 1.1 Hz, 1H), 7.95 (s, 1H), 7.33 − 7.27 (m, 2H), 7.21 (d, J = 5.1 Hz, 1H), 5.47 (s, 2H), 4.89 − 4.78 (m, 4H), 4.73 (s, 2H), 2.69 − 2.61 (m, 1H), 2.58 − 2.53 (m, 1H), 2.47 (s, 3H), 2.41 (s, 3H), 1.98-1.93 (m, 1H), 1.58-1.50 (m, 2H), 0.98 − 0.90 (m, 2H), 0.69 − 0.61 (m, 2H). |
| | (1S,2S)-N-(6-((6-cyclopropyl-8-(6- | |
| | oxo-2,5-dioxa-7- | |
| | azaspiro[3.4]octan-7- | |
| | yl)imidazo[1,2-a]pyridin-2- | |
| | yl)methoxy)-2-methylpyrimidin-4- | |
| | yl)-2-(4-methylpyrimidin-2- | |
| | yl)cyclopropane-1-carboxamide (I- | |
| | 553) | |
| Example 554 | 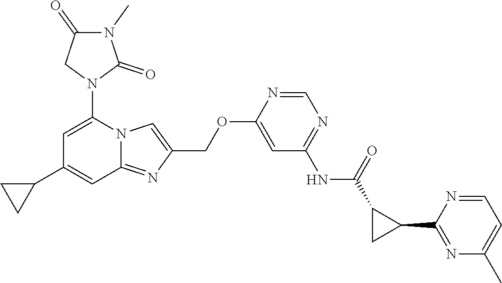 [SMILES: Cc1ccnc([C@H]2C[C@@H]2C(=O)Nc2cc(OCc3cn4c(N5CC(=O)N(C)C5=O)cc(C5CC5)cc4n3)ncn2)n1] | 1-(2-(((6-chloropyrimidin-4-yl)oxy)methyl)- 7-cyclopropylimidazo[1,2-a]pyridin-5-yl)-3- methylimidazolidine-2,4-dione ESI-MS (M + H)+: 554.1, ¹H NMR (400 MHz, DMSO) δ 11.19 (s, 1H), 8.55 (d, J = 0.7 Hz, 1H), 8.53 (d, J = 5.1 Hz, 1H), 7.99 (s, 1H), 7.48 (d, J = 0.7 Hz, 1H), 7.34 (s, 1H), 7.22 (d, J = 5.1 Hz, 1H), 6.82 (d, J = 1.4 Hz, 1H), 5.46 (s, 2H), 4.51 (s, 2H), 2.97 (s, 3H), 2.69 − 2.63 (m, 1H), 2.59 − 2.54 (m, 1H), 2.42 (s, 3H), 2.07-1.97 (m, 1H), 1.60 − 1.50 (m, 2H), 1.07-0.98 (m, 2H), 0.83 − 0.73 (m, 2H). |
| | (1S,2S)-N-(6-((7-cyclopropyl-5-(3- | |
| | methyl-2,4-dioxoimidazolidin-1- | |
| | yl)imidazo[1,2-a]pyridin-2- | |
| | yl)methoxy)pyrimidin-4-yl)-2-(4- | |
| | methylpyrimidin-2- | |
| | yl)cyclopropane-1-carboxamide (I- | |
| | 554) | |
| Example 555 | 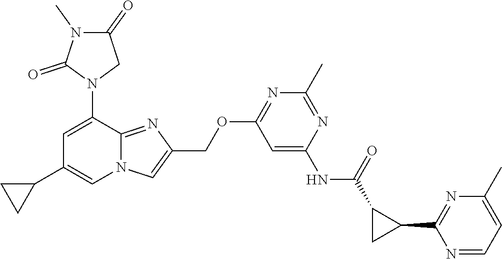 [SMILES: Cc1ccnc([C@H]2C[C@@H]2C(=O)Nc2cc(OCc3cn4cc(C5CC5)cc(N5CC(=O)N(C)C5=O)c4n3)nc(C)n2)n1] | 1-(2-(((6-chloro-2-methylpyrimidin-4- yl)oxy)methyl)-6-cyclopropylimidazo[1,2- a]pyridin-8-yl)-3-methylimidazolidine-2,4- dione ESI-MS (M + H)+: 568.2, ¹H NMR (400 MHz, DMSO) δ 11.17 (s, 1H), 8.53 (d, J = 5.1 Hz, 1H), 8.28 (d, J = 1.1 Hz, 1H), 7.95 (s, 1H), 7.38 (d, J = 1.4 Hz, 1H), 7.31 (s, 1H), 7.21 (d, J = 5.1 Hz, 1H), 5.46 (s, 2H), 4.90 (s, 2H), 2.97 (s, 3H), 2.66 − 2.61 (m, 1H), 2.57 − 2.54 (m, 1H), 2.46 (s, 3H), 2.41 (s, 3H), 1.98 − 1.94 (m, 1H), 1.58 − 1.51 (m, 2H), 0.98 − 0.93 (m, 2H), 0.68 − 0.64 (m, 2H). |
| | (1S,2S)-N-(6-((6-cyclopropyl-8-(3- | |
| | methyl-2,4-dioxoimidazolidin-1- | |
| | yl)imidazo[1,2-a]pyridin-2- | |
| | yl)methoxy)-2-methylpyrimidin-4- | |
| | yl)-2-(4-methylpyrimidin-2- | |
| | yl)cyclopropane-1-carboxamide (I- | |
| | 555) | |
| Example 556 | 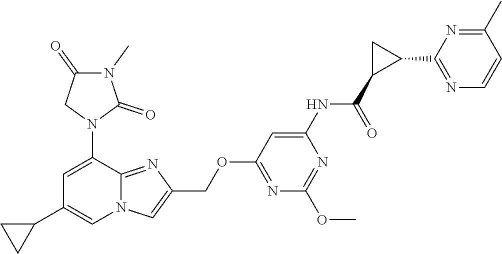 [SMILES: COc1nc(NC(=O)[C@H]2C[C@@H]2c2nccc(C)n2)cc(OCc2cn3cc(C4CC4)cc(N4CC(=O)N(C)C4=O)c3n2)n1] | 1-(2-(((6-chloro-2-methoxypyrimidin-4- yl)oxy)methyl)-6-cyclopropylimidazo[1,2- a]pyridin-8-yl)-3-methylimidazolidine-2,4- dione ESI-MS (M + H)+: 584.2, ¹H NMR (400 MHz, DMSO) δ 11.09 (s, 1H), 8.53 (d, J = 5.1 Hz, 1H), 8.29 (s, 1H), 7.95 (s, 1H), 7.38 (d, J = 1.5 Hz, 1H), 7.21 (d, J = 5.1 Hz, 1H), 7.14 (s, 1H), 5.46 (s, 2H), 4.89 (s, 2H), 3.89 (s, 3H), 2.97 (s, 3H), 2.70 − 2.64 (m, 2H), 2.41 (s, 3H), 1.98 − 1.95 (m, 1H), 1.58 − 1.52 (m, 2H), 0.98 − 0.93 (m, 2H), 0.70 − 0.64 (m, 2H). ESI-MS [M + H]+: 581, |
| | (1S,2S)-N-(6-((6-cyclopropyl-8-(3- | |
| | methyl-2,4-dioxoimidazolidin-1- | |
| | yl)imidazo[1,2-a]pyridin-2- | |
| | yl)methoxy)-2-methoxypyrimidin- | |
| | 4-yl)-2-(4-methylpyrimidin-2- | |
| | yl)cyclopropane-1-carboxamide (I- | |
| | 556) | |
| Example 557 | 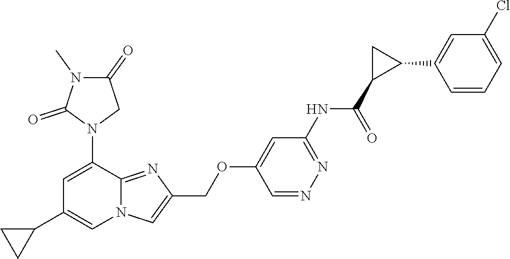 [SMILES: CN1C(=O)CN(c2cc(C3CC3)cn3cc(COc4cnnc(NC(=O)[C@H]5C[C@@H]5c5cccc(Cl)c5)c4)nc23)C1=O] | 1-(2-(((6-chloropyridazin-4-yl)oxy)methyl)- 6-cyclopropylimidazo[1,2-a]pyridin-8-yl)-3- methylimidazolidine-2,4-dione ESI-MS [M + H]+: 572.2, ¹H NMR (400 MHz, DMSO) δ 11.33 (s, 1H), 8.77 (d, J = 2.6 Hz, 1H), 8.45 (s, 1H), 8.30 (s, 1H), 8.26 (d, J = 2.4 Hz, 1H), 8.02 (s, 1H), 7.41 (s, 1H), 7.32 (d, J = 7.9 Hz, 1H), 7.28 (s, 1H), 7.17 (d, J = 7.5 Hz, 1H), 5.37 (s, 2H), 4.93 (s, 2H), 2.98 (s, 3H), 2.45 − 2.38 (m, 2H), 1.98-1.97 (m, 1H), 1.57 − 1.49 (m, 1H), 1.48-1.47 (m, 1H), 0.97-0.95 (m, 2H), 0.67- 0.66 (m, 2H). |
| | (1S,2S)-2-(3-chlorophenyl)-N-(5- | |
| | ((6-cyclopropyl-8-(3-methyl-2,4- | |
| | dioxoimidazolidin-1- | |
| | yl)imidazo[1,2-a]pyridin-2- | |
| | yl)methoxy)pyridazin-3- | |
| | yl)cyclopropane-1-carboxamide (I- | |
| | 557) | |
| Example 558 | 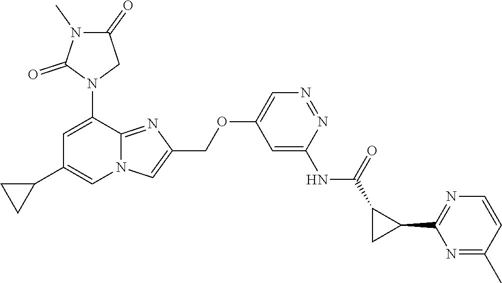 [SMILES: Cc1ccnc([C@H]2C[C@@H]2C(=O)Nc2cc(OCc3cn4cc(C5CC5)cc(N5CC(=O)N(C)C5=O)c4n3)cnn2)n1] | 1-(2-(((6-chloropyridazin-4-yl)oxy)methyl)- 6-cyclopropylimidazo[1,2-a]pyridin-8-yl)-3- methylimidazolidine-2,4-dione ESI-MS [M + H]+: 554.2 ¹H NMR (400 MHz, DMSO) 11.39 (s, 1H), 8.77 (d, J = 2.7 Hz, 1H), 8.54 (d, J = 5.1 Hz, 1H), 8.30 (d, J = 1.3 Hz, 1H), 8.21 (d, J = 2.7 Hz, 1H), 8.02 (s, 1H), 7.41 (d, J = 1.5 Hz, 1H), 7.22 (d, J = 5.1 Hz, 1H), 5.37 (s, 2H), 4.93 (d, J = 4.4 Hz, 2H), 2.98 (s, 3H), 2.72 − 2.66 (m, 1H), 2.60 − 2.55 (m, 1H), 2.42 (s, 3H), 2.00 − 1.95 (m, 1H), 1.61 − 1.52 (m, 2H), 1.01 − 0.92 (m, 2H), 0.71 − 0.63 (m, 2H). |
| | (1S,2S)-N-(5-((6-cyclopropyl-8-(3- | |
| | methyl-2,4-dioxoimidazolidin-1- | |
| | yl)imidazo[1,2-a]pyridin-2- | |
| | yl)methoxy)pyridazin-3-yl)-2-(4- | |
| | methylpyrimidin-2- | |
| | yl)cyclopropane-1-carboxamide (I- | |
| | 558) | |
| Example 559 | 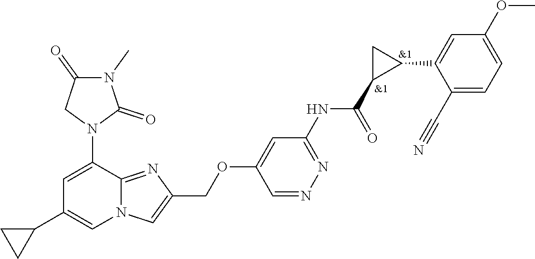 [SMILES: COc1ccc(C#N)c([C@H]2C[C@@H]2C(=O)Nc2cc(OCc3cn4cc(C5CC5)cc(N5CC(=O)N(C)C5=O)c4n3)cnn2)c1] | 1-(2-(((6-chloropyridazin-4-yl)oxy)methyl)- 6-cyclopropylimidazo[1,2-a]pyridin-8-yl)-3- methylimidazolidine-2,4-dione ESI-MS [M + H]+: 593.2. ¹H NMR (400 MHz, DMSO) δ 11.41 (s, 1H), 8.78 (d, J = 2.7 Hz, 1H), 8.30 (s, 1H), 8.26 (d, J = 2.5 Hz, 1H), 8.03 (s, 1H), 7.75 (d, J = 8.6 Hz, 1H), 7.42 (d, J = 1.3 Hz, 1H), 6.99 (dd, J = 8.7, 2.4 Hz, 1H), 6.80 (d, J = 2.4 Hz, 1H), 5.38 (s, 2H), 4.92 (s, 2H), 3.86 (s, 3H), 2.96 (s, 3H), 2.64 − 2.62 (m, 2H), 2.03 − 1.89 (m, 1H), 1.59 (t, J = 7.2 Hz, 2H), 1.01 − 0.89 (m, 2H), 0.75 − 0.60 (m, 2H). |
| | rac-(1S*,2S*)-2-(2-cyano-5- | |
| | methoxyphenyl)-N-(5-((6- | |
| | cyclopropyl-8-(3-methyl-2,4- | |
| | dioxoimidazolidin-1- | |
| | yl)imidazo[1,2-a]pyridin-2- | |
| | yl)methoxy)pyridazin-3- | |
| | yl)cyclopropane-1-carboxamide (I- | |
| | 559) | |
| Example 560 | 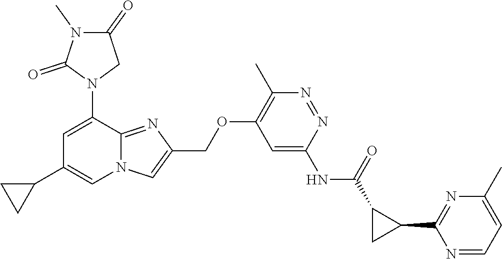 [SMILES: Cc1ccnc([C@H]2C[C@@H]2C(=O)Nc2cc(OCc3cn4cc(C5CC5)cc(N5CC(=O)N(C)C5=O)c4n3)c(C)nn2)n1] | 1-(2-(((6-chloro-3-methylpyridazin-4- yl)oxy)methyl)-6-cyclopropylimidazo[1,2- a]pyridin-8-yl)-3-methylimidazolidine-2,4- dione ESI-MS [M + H]+: 568.2. ¹H NMR (400 MHz, DMSO) δ 11.26 (s, 1H), 8.51 (d, J = 5.1 Hz, 1H), 8.27 (s, 1H), 8.20 (s, 1H), 7.99 (s, 1H), 7.39 (d, J = 1.5 Hz, 1H), 7.19 (d, J = 5.1 Hz, 1H), 5.33 (s, 2H), 4.97 − 4.87 (m, 2H), 2.96 (s, 3H), 2.65 − 2.63 (m, 1H), 2.52 − 2.51 (m, 1H), 2.39 (d, J = 2.5 Hz, 6H), 1.97 − 1.92 (m, 1H), 1.53 (t, J = 9.9 Hz, 2H), 0.94 (t, J = 7.4 Hz, 2H), 0.67 − 0.61 (m, 2H). |
| | (1S,2S)-N-(5-((6-cyclopropyl-8-(3- | |
| | methyl-2,4-dioxoimidazolidin-1- | |
| | yl)imidazo[1,2-a]pyridin-2- | |
| | yl)methoxy)-6-methylpyridazin-3- | |
| | yl)-2-(4-methylpyrimidin-2- | |
| | yl)cyclopropane-1-carboxamide (I- | |
| | 560) | |
| Example 561 | 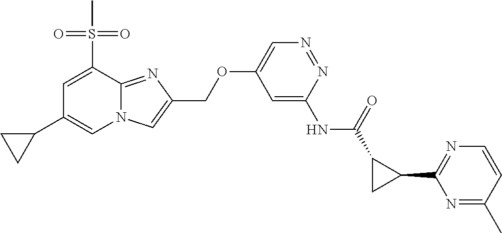 [SMILES: Cc1ccnc([C@H]2C[C@@H]2C(=O)Nc2cc(OCc3cn4cc(C5CC5)cc(S(C)(=O)=O)c4n3)cnn2)n1] | 2-(((6-chloropyridazin-4-yl)oxy)methyl)-6- cyclopropyl-8-(methylsulfonyl)imidazo[1,2- a]pyridine ESI-MS [M + H]+: 520.2. ¹H NMR (400 MHz, DMSO) δ 11.42 (s, 1H), 8.83 (d, J = 2.7 Hz, 1H), 8.71 (d, J = 1.2 Hz, 1H), 8.54 (d, J = 5.1 Hz, 1H), 8.18 − 8.09 (m, 2H), 7.63 (d, J = 1.7 Hz, 1H), 7.23 (d, J = 5.1 Hz, 1H), 5.44 (s, 2H), 3.53 (s, 3H), 2.73 − 2.67 (m, 1H), 2.62 − 2.56 (m, 2H), 2.43 (s, 3H), 2.14 − 2.06 (m, 1H), 1.63 − 1.56 (m, 2H), 1.03 − 0.95 (m, 2H), 0.78 − 0.72 (m, 2H). |
| | (1S,2S)-N-(5-((6-cyclopropyl-8- | |
| | (methylsulfonyl)imidazo[1,2- | |
| | a]pyridin-2-yl)methoxy)pyridazin- | |
| | 3-yl)-2-(4-methylpyrimidin-2- | |
| | yl)cyclopropane-1-carboxamide (I- | |
| | 561) | |

### Example 562

### Synthesis of (1S,2S)—N-(6-((6-cyclopropyl-8-(3-(2-hydroxy-2-methylpropyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide (I-562)

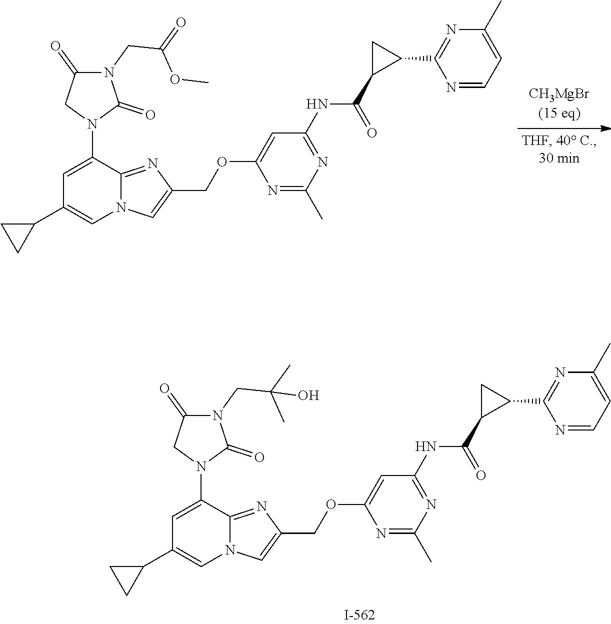 [SMILES: COC(=O)CN1C(=O)CN(c2cc(C3CC3)cn3cc(COc4cc(NC(=O)[C@H]5C[C@@H]5c5nccc(C)n5)nc(C)n4)nc23)C1=O ; Cc1ccnc([C@H]2C[C@@H]2C(=O)Nc2cc(OCc3cn4cc(C5CC5)cc(N5CC(=O)N(CC(C)(C)O)C5=O)c4n3)nc(C)n2)n1]

To a mixture of methyl 2-(3-(6-cyclopropyl-2-(((2-methyl-6-((1S,2S)-2-(4-methylpyrimidin-2-yl)cyclopropane-1-carboxamido)pyrimidin-4-yl)oxy)methyl)imidazo[1,2-a]pyridin-8-yl)-2,5-dioxoimidazolidin-1-yl)acetate (105 mg, 0.17 mmol) in THF (3 mL) was added CH₃MgBr (0.85 mL, 2.55 mmol) at 0° C. The mixture was stirred at 40° C. for 30 min. The mixture was quenched with sat·NH₄Cl (10 mL) and extracted with EtOAc (3×20 mL). Then the combined organic layers were concentrated in vacuo to give the crude product, which was purified by preparative TLC (eluent: DCM/MeOH=12/1) to give (1S,2S)—N-(6-((6-cyclopropyl-8-(3-(2-hydroxy-2-methylpropyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide (3.4 mg, 3%) as a white solid. ESI-MS (M+H)+: 626.3, ¹H NMR (400 MHz, MeOD) δ 8.46 (d, J=5.2 Hz, 1H), 8.23 (s, 1H), 7.90 (s, 1H), 7.39 (s, 1H), 7.33 (d, J=1.4 Hz, 1H), 7.17 (d, J=5.2 Hz, 1H), 5.51 (s, 2H), 4.69 (s, 2H), 3.63 (s, 2H), 2.73-2.68 (m, 1H), 2.56-2.51 (m, 1H), 2.47 (d, J=1.5 Hz, 6H), 2.03-1.94 (m, 2H), 1.67-1.63 (m, 1H), 1.27 (s, 6H), 1.02-0.97 (m, 2H), 0.77-0.73 (m, 2H).

### Example 563

### Synthesis of (1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide (I-563)

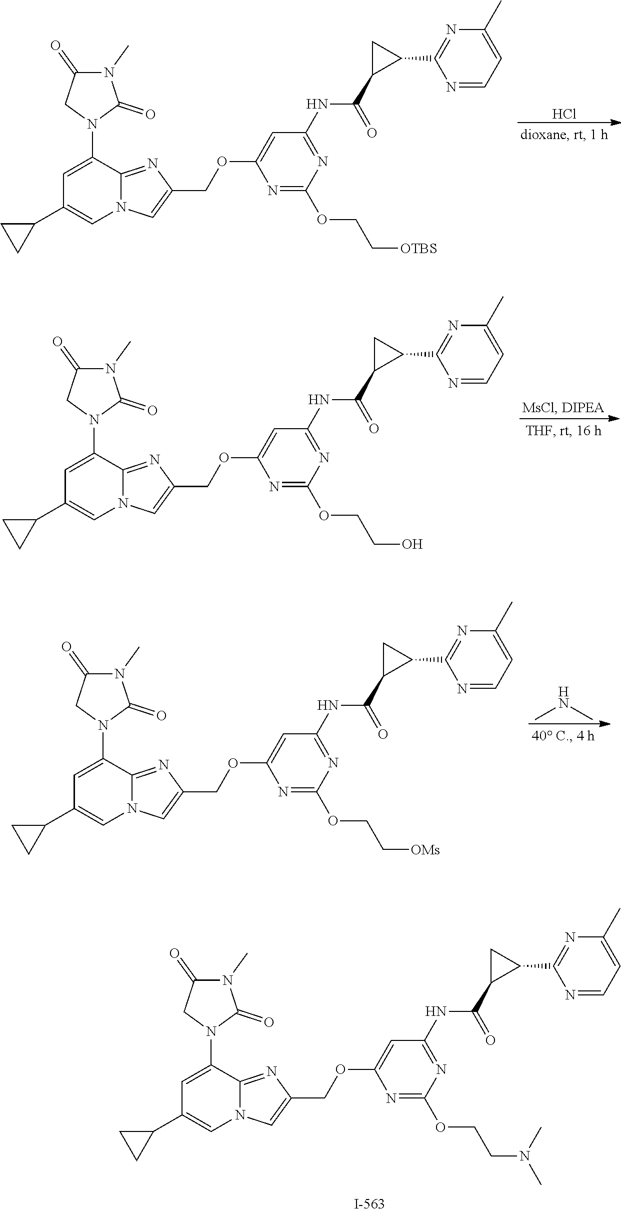

Synthesis of (1S,2S)—N-(6-((6-cyclopropyl-8-(3-methyl-2,4-dioxoimidazolidin-1-yl)imidazo[1,2-a]pyridin-2-yl)methoxy)-2-(2-hydroxyethoxy)pyrimidin-4-yl)-2-(pyrimidin-2-yl)cyclopropane-1-carboxamide. To the mixture of (1S,2S)—N-(2-(2-((tert-butyldimethylsilyl)oxy)ethoxy)-6-((6-cyclopropyl-8-(3-meth yl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (90 mg, 0.13 mmol) (prepared in a similar manner to methyl 2-(3-(6-cyclopropyl-2-(((2-methyl-6-((1S,2S)-2-(4-methylpyrimidin-2-yl)cyclopropane-1-carboxamido)pyrimidin-4-yl)oxy)methyl)imidazo[1,2-a]pyridin-8-yl)-2,5-dioxoimidazolidin-1-yl)acetate) in dioxane (5 mL) was added HCl/dioxane (4M solution in dioxane, 3 mL). The reaction mixture was stirred at room temperature for 1 h, concentrated in vacuo, then diluted in DCM/MeOH (30 mL, 10/1) and adjusted to pH=9˜10 by aqueous NaHCO₃ (10 mL). The organics were washed with brine (30 mL), dried over Na₂SO₄, and concentrated in vacuo to give the crude, which was purified by silica gel chromatography (eluent: DCM/MeOH=10/1) to afford (1S,2S)—N-(6-((6-cyclopropyl-8-(3-methyl-2,4-dioxoimidazolidin-1-yl)imidazo[1,2-a]pyridin-2-yl)methoxy)-2-(2-hydroxyethoxy)pyrimidin-4-yl)-2-(pyrimidin-2-yl)cyclopropane-1-carboxamide (60 mg, 79%) as yellow solid. ESI-MS [M+H]+: 614.2.

Synthesis of 2-((4-((6-cyclopropyl-8-(3-methyl-2,4-dioxoimidazolidin-1-yl)imidazo[1,2-a]pyridin-2-yl)methoxy)-6-((1S,2S)-2-(4-methylpyrimidin-2-yl)cyclopropane-1-carboxamido)pyrimidin-2-yl)oxy)methylmethanesulfonate. To a mixture of (1S,2S)—N-(6-((6-cyclopropyl-8-(3-methyl-2,4-dioxoimidazolidin-1-yl)imidazo[1,2-a]pyridin-2-yl)methoxy)-2-(2-hydroxyethoxy)pyrimidin-4-yl)-2-(4-methylpyrimidin-2-yl)cyclopropane-1-carboxamide (30 mg, 0.049 mmol) in THF (2 mL) was added DIPEA (19 mg, 0.147 mmol) and MsCl (14 mg, 0.122 mmol) at 0° C. The reaction mixture was stirred at room temperature for 16 h. The reaction was then quenched with water (10 mL) and extracted with EtOAc (3×10 mL). The combined organic layers were washed with brine (10 mL), dried over anhydrous Na₂SO₄, and concentrated in vacuo to afford 2-((4-((6-cyclopropyl-8-(3-methyl-2,4-dioxoimidazolidin-1-yl)imidazo[1,2-a]pyridin-2-yl)methoxy)-6-((1S,2S)-2-(4-methylpyrimidin-2-yl)cyclopropane-1-carboxamido)pyrimidin-2-yl)oxy)ethyl methanesulfonate (30 mg, 87%) as a yellow solid, which was used in the next step without purification. ESI-MS [M+H]+: 692.2.

Synthesis of (1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide (I-563). A mixture of 2-((4-((6-cyclopropyl-8-(3-methyl-2,4-dioxoimidazolidin-1-yl)imidazo[1,2-a]pyridin-2-yl)methoxy)-6-((1S,2S)-2-(4-methylpyrimidin-2-yl)cyclopropane-1-carboxamido)pyrimidin-2-yl)oxy)ethyl methanesulfonate (30 mg, 0.043 mmol) in dimethylamine (1.5 mL) was stirred at 40° C. for 4 h. The reaction mixture was concentrated in vacuo and purified by preparative TLC (eluent: DCM/MeOH=15/1) to afford (1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide (8 mg, 30%) as a pale solid. ESI-MS (M+H)+: 641.2, ¹H NMR (400 MHz, DMSO) δ 11.03 (s, 1H), 8.53 (d, J=5.1 Hz, 1H), 8.28 (d, J=1.4 Hz, 1H), 7.93 (s, 1H), 7.38 (d, J=1.5 Hz, 1H), 7.21 (d, J=5.1 Hz, 1H), 7.13 (s, 1H), 5.45 (s, 2H), 4.89 (s, 2H), 4.35 (t, J=5.9 Hz, 2H), 2.97 (s, 3H), 2.67-2.63 (m, 1H), 2.59-2.54 (m, 3H), 2.42 (s, 3H), 2.17 (s, 6H), 1.98-1.95 (m, 1H), 1.57-1.52 (m, 2H), 0.98-0.93 (m, 2H), 0.68-0.64 (m, 2H).

The compound in Table 42 was prepared in a similar manner to (1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide.

TABLE 42

| Example | Structure | Analytical Data |
|---|---|---|
| Example 564 | 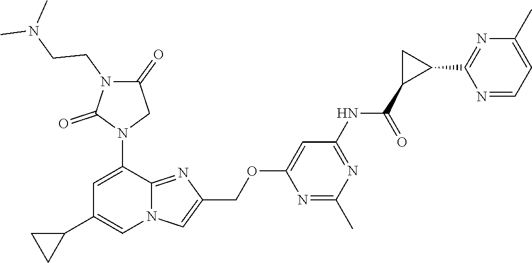 | ESI-MS (M + H)+: 625.3, ¹H NMR (400 MHz, DMSO) δ 11.17 (s, 1H), 8.53 (d, J = 5.1 Hz, 1H), 8.28 (d, J = 1.5 Hz, 1H), 7.95 (s, 1H), 7.37 (d, J = 1.5 Hz, 1H), 7.31 (s, 1H), 7.21 (d, J = 5.1 Hz, 1H), 5.47 (s, 2H), 4.93 (s, 2H), 3.60 − 3.57 (m, 2H), 2.67 − 2.62 (m, 1H), 2.57 − 2.54 (m, 1H), 2.47 − 2.45 (m, 5H), 2.41 (s, 3H), 2.18 (s, 6H), 1.99 − 1.95 (m, 1H), 1.58 − 1.51 (m, 2H), 0.98 − 0.93 (m, 2H), 0.68 − 0.65 (m, 2H). |
| | (1S,2S)-N-(6-((6-cyclopropyl-8- | |
| | (3-(2-(dimethylamino)ethyl)- | |
| | 2,4-dioxoimidazolidin-1- | |
| | yl)imidazo[1,2-a]pyridin-2- | |
| | yl)methoxy)-2-methylpyrimidin- | |
| | 4-yl)-2-(4-methylpyrimidin-2- | |
| | yl)cyclopropane-1-carboxamide | |
| | (I-564) | |

### Synthesis of 2-(((2-bromopyridin-4-yl)oxy)methyl)-5-cyclopropyl-7-(4-methylpiperazin-1-yl)pyrazolo[1,5-a]pyridine

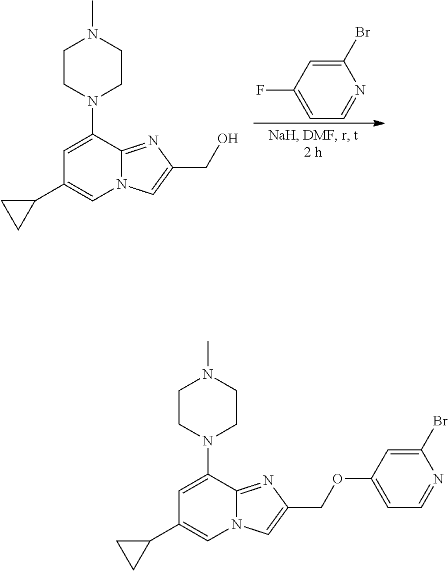 [SMILES: CN1CCN(c2cc(C3CC3)cn3cc(CO)nc23)CC1 ; Fc1ccnc(Br)c1 ; CN1CCN(c2cc(C3CC3)cn3cc(COc4ccnc(Br)c4)nc23)CC1]

A mixture of (6-cyclopropyl-8-(4-methylpiperazin-1-yl)imidazo[1,2-a]pyridin-2-yl)methanol (80 mg, 0.28 mmol), 2-bromo-4-fluoropyridine (54 mg, 0.31 mmol), NaH (33.5 mg, 1.4 mmol) and DMF (4 mL) was stirred at room temperature for 2 h. Water (20 mL) was added to the mixture, which was then extracted with EtOAc (3×20 mL). The combined organic layers were washed with brine, dried over Na₂SO₄, filtered, and concentrated in vacuo to give the crude, which was purified by silica gel chromatography (DCM/MeOH=20/1) to give 2-(((2-bromopyridin-4-yl)oxy)methyl)-6-cyclopropyl-8-(4-methylpiperazin-1-yl)imidazo[1,2-a]pyridine (80 mg, 65% ) as a gray solid. ESI-MS [M+H]+: 442.2.

The compounds in Table 43 were prepared in a similar manner to 2-(((2-bromopyridin-4-yl)oxy)methyl)-5-cyclopropyl-7-(4-methylpiperazin-1-yl)pyrazolo[1,5-a]pyridine.

TABLE 43

| | Analytical |
|---|---|
| Structure | Data |
| 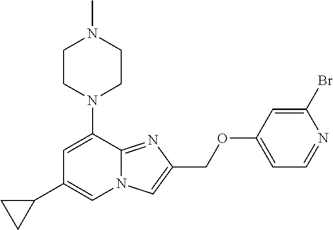 [SMILES: CN1CCN(c2cc(C3CC3)cn3cc(COc4ccnc(Br)c4)nc23)CC1] | ESI-MS [M + H]+: 442.2. |
| 2-(((2-bromopyridin-4-yl)oxy) | |
| methyl)-6-cyclopropyl-8-(4- | |
| methylpiperazin-1-yl)imidazo[1,2-a]pyridine | |
| 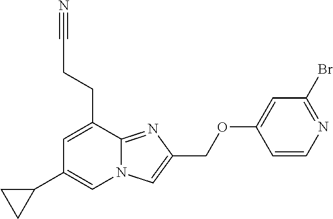 [SMILES: N#CCCc1cc(C2CC2)cn2cc(COc3ccnc(Br)c3)nc12] | ESI-MS [M + H]+: 397.2. |
| 3-(2-(((2-bromopyridin-4-yl)oxy)methyl)- | |
| 6-cyclopropylimidazo[1,2- | |
| a]pyridin-8-yl)propanenitrile | |
| 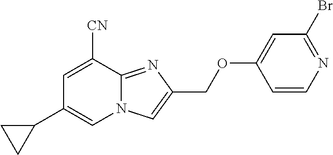 [SMILES: N#Cc1cc(C2CC2)cn2cc(COc3ccnc(Br)c3)nc12] | ESI-MS [M + H]+: 369.1. |
| 2-(((2-bromopyridin-4-yl)oxy)methyl)- | |
| 6-cyclopropylimidazo[1,2- | |
| a]pyridine-8-carbonitrile | |
| 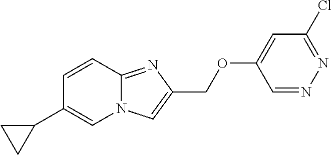 [SMILES: Clc1cc(OCc2cn3cc(C4CC4)ccc3n2)cnn1] | ESI-MS [M + H]+: 301.2. |
| 2-(((6-chloropyridazin-4-yl)oxy)methyl)- | |
| 6-cyclopropylimidazo[1,2- | |
| a]pyridine | |
| 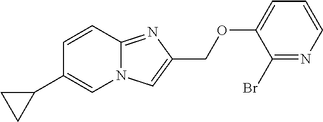 [SMILES: Brc1ncccc1OCc1cn2cc(C3CC3)ccc2n1] | ESI-MS [M + H]+: 344.2. |
| 2-(((2-bromopyridin-3-yl)oxy)methyl)- | |
| 6-cyclopropylimidazo[1,2- | |
| a]pyridine | |
| 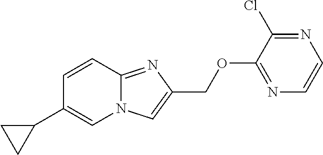 [SMILES: Clc1nccnc1OCc1cn2cc(C3CC3)ccc2n1] | ESI-MS [M + H]+: 301.2. |
| 2-(((3-chloropyrazin-2-yl)oxy)methyl)- | |
| 6-cyclopropylimidazo[1,2- | |
| a]pyridine | |
| 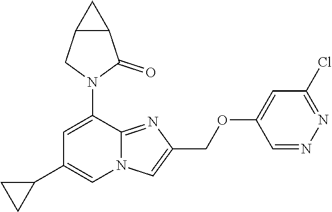 [SMILES: O=C1C2CC2CN1c1cc(C2CC2)cn2cc(COc3cnnc(Cl)c3)nc12] | ESI-MS [M + H]+: 396.2. |
| 3-(2-(((6-chloropyridazin-4-yl)oxy)methyl)-6- | |
| cyclopropylimidazo[1,2-a]pyridin-8-yl)- | |
| 3-azabicyclo[3.1.0]hexan-2- | |
| one | |

### Example 565

### Synthesis of (1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide (I-565)

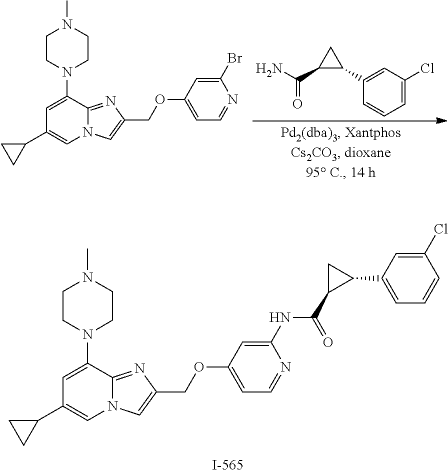 [SMILES: CN1CCN(c2cc(C3CC3)cn3cc(COc4ccnc(Br)c4)nc23)CC1 ; NC(=O)[C@H]1C[C@@H]1c1cccc(Cl)c1 ; CN1CCN(c2cc(C3CC3)cn3cc(COc4ccnc(NC(=O)[C@H]5C[C@@H]5c5cccc(Cl)c5)c4)nc23)CC1]

To a mixture of 2-(((2-bromopyridin-4-yl)oxy)methyl)-6-cyclopropyl-8-(4-methylpiperazin-1-yl)imidazo[1,2-a]pyridine (60 mg, 0.135 mmol), (1S,2S)-2-(3-chlorophenyl)cyclopropane-carboxamide (26.3 mg, 0.135 mmol), and Cs₂CO₃ (131.9 mg, 0.405 mmol) in 1,4-dioxane (5 mL) was added Pd₂(dba)₃ (18.3 mg, 0.02 mmol) and xantphos (15.6 mg, 0.027 mmol). After stirring at 95° C. for 6 h under N₂, the reaction mixture was filtered through Celite© and the filter cake was washed with DCM/MeOH (10/1, 20 mL). The filtrate was concentrated in vacuo to give the crude, which was purified by preparative TLC (DCM/MeOH=20/1) to give to (1S,2S)-2-(3-chlorophenyl)-N-(4-((6-cyclopropyl-8-(4-methylpiperazin-1-yl)imidazo[1,2-a]pyridin-2-yl)methoxy)pyridin-2-yl)cyclopropane-1-carboxamide (20 mg, 27%) as a white solid. ESI-MS [M+H]+: 557.2, ¹H NMR (400 MHz, DMSO) δ 10.74 (s, 1H), 8.12 (d, J=5.8 Hz, 1H), 7.91 (s, 1H), 7.83 (s, 1H), 7.80 (s, 1H), 7.36-7.29 (m, 1H), 7.29-7.22 (m, 2H), 7.16 (d, J=7.6 Hz, 1H), 6.84-6.82 (m, 1H), 6.18 (s, 1H), 5.22 (s, 2H), 2.45-2.40 (m, 4H), 2.53-2.50 (m, 4H), 2.47-2.35 (m, 2H), 2.23 (s, 3H), 1.90-1.84 (m, 1H), 1.56-1.47 (m, 1H), 1.43-1.39 (m, 1H), 0.94-0.84 (m, 2H), 0.74-0.66 (m, 2H).

The compounds in Table 44 were prepared in a similar manner to (1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide from an appropriate cyclopropyl amide and the indicated coupling partner.

TABLE 44

| | | Coupling Partner/ |
|---|---|---|
| Example | Structure | Analytical Data |
| Example 566 | 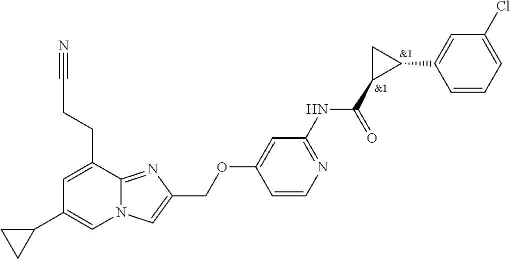 [SMILES: N#CCCc1cc(C2CC2)cn2cc(COc3ccnc(NC(=O)[C@H]4C[C@@H]4c4cccc(Cl)c4)c3)nc12] | 3-(2-(((2-bromopyridin-4- yl)oxy)methyl)-6- cyclopropylimidazo[1,2-a]pyridin-8- yl)propanenitrile ESI-MS [M + H]+: 512.2, ¹H NMR (400 MHz, DMSO) δ 10.75 (s, 1H), 8.29 (s, 1H), 8.12 (d, J = 5.8 Hz, 1H), 7.91 (s, 1H), 7.84 (d, J = 1.8 Hz, 1H), 7.38 − 7.28 (m, 1H), 7.29 − 7.23 (m, 2H), 7.16 (d, J = 7.7 Hz, 1H), 6.97 (s, 1H), 6.83 − 6.82 (m, 1H), 5.25 (s, 2H), 3.16 (t, J = 7.0 Hz, 2H), 3.06 (t, J = 6.8 Hz, 2H), 2.45 − 2.35 (m, 2H), 1.96 − 1.91 (m, 1H), 1.53 − 1.46 (m, 1H), 1.45 − 1.35 (m, 1H), 0.99 − 0.91 (m, 2H), 0.73 − 0.68 (m, 2H). |
| | rac-(1S*,2S*)-2-(3-chlorophenyl)-N- | |
| | (4-((8-(2-cyanoethyl)-6- | |
| | cyclopropylimidazo[1,2-a]pyridin-2- | |
| | yl)methoxy)pyridin-2- | |
| | yl)cyclopropane-1-carboxamide (I- | |
| | 566) | |
| Example 567 | 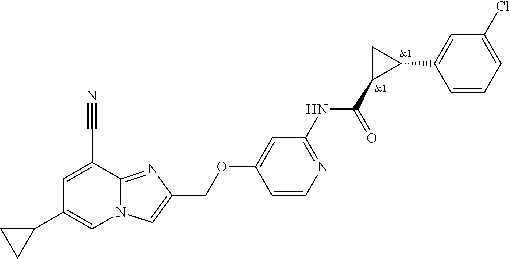 [SMILES: N#Cc1cc(C2CC2)cn2cc(COc3ccnc(NC(=O)[C@H]4C[C@@H]4c4cccc(Cl)c4)c3)nc12] | 2-(((2-bromopyridin-4-yl)oxy)methyl)-6- cyclopropylimidazo[1,2-a]pyridine-8- carbonitrile ESI-MS [M + H]⁺: 484.2. ¹H NMR (400 MHz, DMSO) δ 10.76 (s, 1H), 8.71 (s, 1H), 8.14 (d, J = 5.7 Hz, 1H), 8.10 (d, J = 27.8 Hz, 1H), 7.81 (d, J = 9.0 Hz, 2H), 7.37 − 7.22 (m, 3H), 7.16 (d, J = 7.6 Hz, 1H), 6.84 (d, J = 7.6 Hz, 1H), 5.30 (s, 2H), 2.42 − 2.40 (m, 2H), 2.07 − 1.90 (m, 1H), 1.59 − 1.47 (m, 1H), 1.43 − 1.37 (m, 1H), 0.99 − 0.94 (m, 2H), 0.80 − 0.76 (m, 2H). |
| | rac-(1S*,2S*)-2-(3-chlorophenyl)-N- | |
| | (4-((8-cyano-6- | |
| | cyclopropylimidazo[1,2-a]pyridin-2- | |
| | yl)methoxy)pyridin-2- | |
| | yl)cyclopropane-1-carboxamide (I- | |
| | 567) | |
| Example 568 | 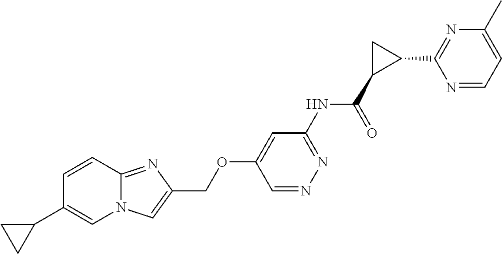 [SMILES: Cc1ccnc([C@H]2C[C@@H]2C(=O)Nc2cc(OCc3cn4cc(C5CC5)ccc4n3)cnn2)n1] | 2-(((6-chloropyridazin-4-yl)oxy)methyl)- 6-cyclopropylimidazo[1,2-a]pyridine ESI-MS [M + H]+: 442.2 ¹H NMR (400 MHz, DMSO) δ 11.41 (s, 1H), 8.81 (d, J = 2.7 Hz, 1H), 8.54 (d, J = 5.1 Hz, 1H), 8.37 (s, 1H), 8.05 (d, J = 2.7 Hz, 1H), 7.93 (s, 1H), 7.44 (d, J = 9.4 Hz, 1H), 7.22 (d, J = 5.1 Hz, 1H), 7.02 (dd, J = 9.4, 1.7 Hz, 1H), 5.34 (s, 2H), 2.73 − 2.66 (m, 1H), 2.64 − 2.56 (m, 1H), 2.43 (s, 3H), 2.00 − 1.90 (m, 1H), 1.63 − 1.53 (m, 2H), 0.96 − 0.89 (m, 2H), 0.74 − 0.63 (m, 2H). |
| | (1S,2S)-N-(5-((6- | |
| | cyclopropylimidazo[1,2-a]pyridin-2- | |
| | yl)methoxy)pyridazin-3-yl)-2-(4- | |
| | methylpyrimidin-2-yl)cyclopropane- | |
| | 1-carboxamide (I-568) | |
| Example 569 | 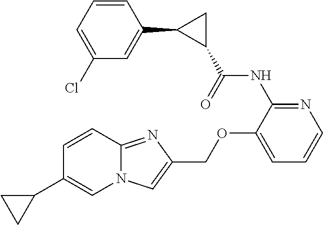 [SMILES: O=C(Nc1ncccc1OCc1cn2cc(C3CC3)ccc2n1)[C@H]1C[C@@H]1c1cccc(Cl)c1] | 2-(((2-bromopyridin-3-yl)oxy)methyl)-6- cyclopropylimidazo[1,2-a]pyridine ESI-MS [M + H]+: 459.1. ¹H NMR (400 MHz, DMSO) δ 9.98 (s, 1H), 8.33 (s, 1H), 7.94 (d, J = 3.9 Hz, 1H), 7.84 (s, 1H), 7.65 (d, J = 7.5 Hz, 1H), 7.41 (d, J = 9.3 Hz, 1H), 7.34 − 7.15 (m, 4H), 7.08 (d, J = 7.3 Hz, 1H), 6.99 (dd, J = 9.4 Hz, 1.4 Hz, 1H), 5.24 (s, 2H), 2.38 − 2.27 (m, 2H), 1.95 − 1.91 (m, 1H), 1.48 − 1.44 (m, 1H), 1.39 − 1.35 (m, 1H), 0.95 − 0.90 (m, 2H), 0.70 − 0.66 (m, 2H). |
| | (1S,2S)-2-(3-chlorophenyl)-N-(3-((6- | |
| | cyclopropylimidazo[1,2-a]pyridin-2- | |
| | yl)methoxy)pyridin-2- | |
| | yl)cyclopropane-1-carboxamide (I- | |
| | 569) | |
| Example 570 | 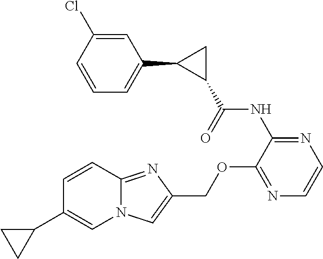 [SMILES: O=C(Nc1nccnc1OCc1cn2cc(C3CC3)ccc2n1)[C@H]1C[C@@H]1c1cccc(Cl)c1] | 2-(((3-chloropyrazin-2-yl)oxy)methyl)- 6-cyclopropylimidazo[1,2-a]pyridine ESI-MS [M + H]⁺: 460.1. ¹H NMR (400 MHz, DMSO-d6) δ 10.30 (s, 1H), 8.33 (s, 1H), 8.02 (dd, J = 8.4, 2.8 Hz, 2H), 7.84 (s, 1H), 7.41 (d, J = 9.3 Hz, 1H), 7.31 − 7.22 (m, 2H), 7.22 − 7.20 (m, 1H), 7.10 (dd, J= 7.3, 1.5 Hz, 1H), 6.99 (dd, J = 9.4, 1.7 Hz, 1H), 5.49 (s, 2H), 2.40 − 2.30 (m, 2H), 1.97 − 1.89 (m, 1H), 1.51 − 1.45 (m, 1H), 1.43 − 1.37 (m, 1H), 0.95 − 0.89 (m, 2H), 0.71 − 0.65 (m, 2H). |
| | (1S,2S)-2-(3-chlorophenyl)-N-(3-((6- | |
| | cyclopropylimidazo[1,2-a]pyridin-2- | |
| | yl)methoxy)pyrazin-2- | |
| | yl)cyclopropane-1-carboxamide (I- | |
| | 570) | |
| Example 571 | 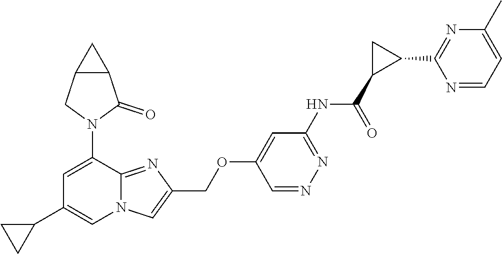 [SMILES: Cc1ccnc([C@H]2C[C@@H]2C(=O)Nc2cc(OCc3cn4cc(C5CC5)cc(N5CC6CC6C5=O)c4n3)cnn2)n1] | 3-(2-(((6-chloropyridazin-4- yl)oxy)methyl)-6- cyclopropylimidazo[1,2-a]pyridin-8-yl)- 3-azabicyclo[3.1.0]hexan-2-one ESI-MS [M + H]⁺: 537.3, ¹H NMR (400 MHz, DMSO) δ 11.41 (s, 1H), 8.79 (d, J = 2.6 Hz, 1H), 8.54 (d, J = 5.1 Hz, 1H), 8.28 (s, 1H), 8.18-8.15 (m, 1H), 7.97 (s, 1H), 7.22 (d, J = 5.1 Hz, 1H), 7.11 (s, 1H), 5.35 (s, 2H), 4.35-4.29 (m, 1H), 4.02 (d, J = 9.8 Hz, 1H), 2.74 − 2.66 (m, 1H), 2.65 − 2.58 (m, 1H), 2.43 (s, 3H), 2.12-2.05 (m, 1H), 2.03-1.97 (m, 1H), 1.95-1.89 (m, 1H), 1.60-1.54 (m, 2H), 1.22-1.14 (m, 1H), 0.97 − 0.89 (m, 2H), 0.86-0.80 (m, 1H), 0.68-0.65 (m, 2H). |
| | (1S,2S)-N-(5-((6-cyclopropyl-8-(2- | |
| | oxo-3-azabicyclo[3.1.0]hexan-3- | |
| | yl)imidazo[1,2-a]pyridin-2- | |
| | yl)methoxy)pyridazin-3-yl)-2-(4- | |
| | methylpyrimidin-2-yl)cyclopropane- | |
| | 1-carboxamide (I-571) | |

### Example 572

### Synthesis of rac-(1S*,2S*)—N-(4-((8-(aminomethyl)-6-cyclopropylimidazo[1,2-a]pyridin-2-yl)methoxy)pyridin-2-yl)-2(3-chlorophenyl)cyclopropane-1-carboxamide (I-572)

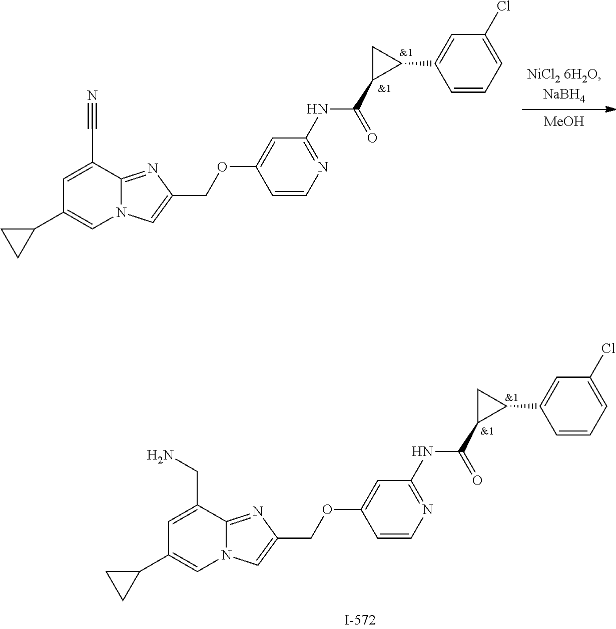 [SMILES: "MeOH" ; N#Cc1cc(C2CC2)cn2cc(COc3ccnc(NC(=O)[C@H]4C[C@@H]4c4cccc(Cl)c4)c3)nc12 ; NCc1cc(C2CC2)cn2cc(COc3ccnc(NC(=O)[C@H]4C[C@@H]4c4cccc(Cl)c4)c3)nc12]

The title compound was prepared from rac-(1S*,2S*)-2-(3-chlorophenyl)-N-(4-((8-cyano-6-cyclopropylimidazo[1,2-a]pyridin-2-yl)methoxy)pyridin-2-yl)cyclopropane-1-carboxamide using a similar method to that used for the synthesis of (1S,2S)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide. ESI-MS [M+H]+: 488.2, ¹H NMR (400 MHz, DMSO) δ 10.76 (s, 1H), 8.32 (s, 1H), 8.13 (d, J=5.6 Hz, 1H), 7.93 (s, 1H), 7.84 (s, 1H), 7.34-7.27 (m, 3H), 7.16 (d, J=7.5 Hz, 1H), 7.06 (s, 1H), 6.82 (d, J=4.0 Hz, 1H), 5.25 (s, 2H), 4.13 (s, 2H), 2.47-2.33 (m, 2H), 2.00-1.83 (m, 1H), 1.58-1.46 (m, 1H), 1.45-1.36 (m, 1H), 1.05-0.82 (m, 2H), 0.82-0.50 (m, 2H).

### Example 573

### Synthesis of (1S,2S)—N-(4-((6-cyclopropyl-8-(3-methyl-2,4-dioxoimidazolidin-1-yl)imidazo[1,2-a]pyridin-2-yl)methoxy)-6-methyl-1,3,5-triazin-2-yl)-2-(4-methylpyrimidin-2-yl)cyclopropane-1-carboxamide (I-573)

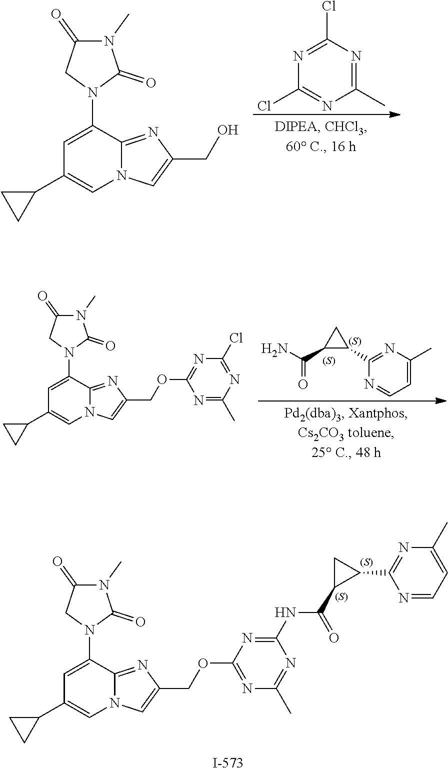

Synthesis of 1-(2-(((4-chloro-6-methyl-1,3,5-triazin-2-yl)oxy)methyl)-6-cyclopropylimidazo[1,2-a]pyridin-8-yl)-3-methylimidazolidine-2,4-dione. To a mixture of 1-(6-cyclopropyl-2-(hydroxymethyl)imidazo[1,2-a]pyridin-8-yl)-3-methylimidazolidine-2,4-dione (200 mg, 0.66 mmol) and 2,4-dichloro-6-methyl-1,3,5-triazine (538 mg, 3.3 mmol) in CHCl₃ (10 mL) was added DIPEA (256 mg, 1.98 mmol). The mixture was stirred at 60° C. for 16 h. Water (20 mL) was added, and the reaction mixture was extracted with DCM (3×20 mL). The combined organic layers were washed with brine, dried over Na₂SO₄, filtered, and concentrated in vacuo to give the crude, which was purified by silica gel chromatography (DCM/MeOH=30/1) to afford 1-(2-(((4-chloro-6-methyl-1,3,5-triazin-2-yl)oxy)methyl)-6-cyclopropylimidazo[1,2-a]pyridin-8-yl)-3-methylimidazolidine-2,4-dione (176 mg, yield 62%) as pale yellow solid. ESI-MS [M+H]+: 428.1.

Synthesis of (1S,2S)—N-(4-((6-cyclopropyl-8-(3-methyl-2,4-dioxoimidazolidin-1-yl)imidazo[1,2-a]pyridin-2-yl)methoxy)-6-methyl-1,3,5-triazin-2-yl)-2-(4-methylpyrimidin-2-yl)cyclopropane-1-carboxamide (I-573). The title compound was prepared in a similar manner to (1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide from 1-(2-(((4-chloro-6-methyl-1,3,5-triazin-2-yl)oxy)methyl)-6-cyclopropylimidazo[1,2-a]pyridin-8-yl)-3-methylimidazolidine-2,4-dione and (1S,2S)-2-(4-methylpyrimidin-2-yl)cyclopropane-1-carboxamide. ESI-MS [M+H]+: 569.2. ¹H NMR (400 MHz, DMSO) δ 11.21 (s, 1H), 8.49 (d, J=5.1 Hz, 1H), 8.28 (d, J=1.2 Hz, 1H), 7.98 (s, 1H), 7.38 (d, J=1.5 Hz, 1H), 7.16 (d, J=5.1 Hz, 1H), 5.44 (s, 2H), 4.88 (s, 2H), 3.02-2.95 (m, 4H), 2.59-2.56 (m, 1H), 2.38-2.37 (m, 6H), 1.99-1.95 (m, 1H), 1.60-1.57 (m, 2H), 0.98-0.93 (m, 2H), 0.69-0.65 (m, 2H).

### Synthesis of rac-(1S*,2S*)—N-(5-aminopyridin-3-yl)-2-(3-chlorophenyl)cyclopropane-1-carboxamide

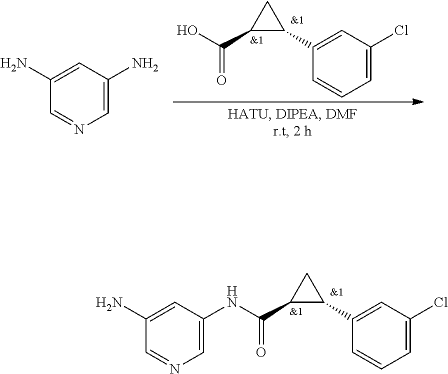 [SMILES: Nc1cncc(N)c1 ; O=C(O)[C@H]1C[C@@H]1c1cccc(Cl)c1 ; Nc1cncc(NC(=O)[C@H]2C[C@@H]2c2cccc(Cl)c2)c1]

A mixture of pyridine-3,5-diamine (30 mg, 0.275 mmol), rac-(1S*,2S*)-2-(3-chlorophenyl)cyclopropane-1-carboxylic acid (54 mg, 0.275 mmol), HATU (125 mg, 0.33 mmol), and DIPEA (178 mg, 1.38 mmol) in DMF (2 mL) was stirred at room temperature for 2 h. The reaction mixture was poured into water (30 mL) and extracted with EtOAc (2×30 mL). The combined organics were washed with water (3×60 mL) and brine (60 mL), dried over anhydrous Na₂SO₄, then concentrated in vacuo to give the crude product, which was purified by silica gel chromatography (eluent: EtOAc) to give rac-(1S*,2S*)—N-(5-aminopyridin-3-yl)-2-(3-chlorophenyl)cyclopropane-1-carboxamide (50 mg, 63%) as a yellow solid. ESI-MS [M+H]⁺: 288.1.

The compound in Table 45 was made in a similar manner to rac-(1S*,2S*)—N-(5-aminopyridin-3-yl)-2-(3-chlorophenyl)cyclopropane-1-carboxamide.

TABLE 45

| Intermediate | Analytical Data |
|---|---|
| 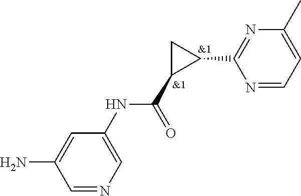 [SMILES: Cc1ccnc([C@H]2C[C@@H]2C(=O)Nc2cncc(N)c2)n1] | ESI-MS [M + H]+: 270.2. |
| rac-(1S*,2S*)-N-(5-aminopyridin-3-yl)-2-(4- | |
| methylpyrimidin-2-yl)cyclopropane-1- | |
| carboxamide | |

### Example 574

### Synthesis of rac-(1S*,2S*)-2-(3-chlorophenyl)-N-(5-(((6-cyclopropylimidazo[1,2-a]pyridin-2-yl)methyl)amino)pyridin-3-yl)cyclopropane-1-carboxamide (I-574)

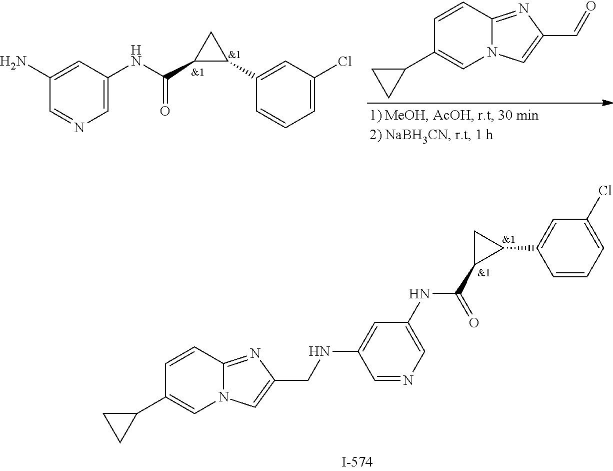

A mixture of rac-(1S*,2S*)—N-(5-aminopyridin-3-yl)-2-(3-chlorophenyl)cyclopropane-1-carboxamide (50 mg, 0.174 mmol), 6-cyclopropylimidazo[1,2-a]pyridine-2-carbaldehyde (32 mg, 0.174 mmol) and AcOH (0.2 mL) in MeOH (5 mL) was stirred at room temperature for 30 min. NaBH₃CN (55 mg, 0.87 mmol) was added to the mixture, which was stirred at room temperature for 1 h. The reaction mixture was poured into water (30 mL), basified to pH=9˜10 with saturated aq·NaHCO₃, and extracted with EtOAc (2×30 mL). The combined organics were washed with brine (60 mL), dried over anhydrous Na₂SO₄, and concentrated in vacuo to give the crude product, which was purified by preparative HPLC to give rac-(1S*,2S*)-2-(3-chlorophenyl)-N-(5-(((6-cyclopropylimidazo[1,2-a]pyridin-2-yl)methyl)amino)pyridin-3-yl)cyclopropane-1-carboxamide (27 mg, 34%) as a pale solid. ESI-MS [M+H]+: 458.2. ¹H NMR (400 MHz, DMSO) δ 10.16 (s, 1H), 8.28 (s, 1H), 7.97 (d, J=1.5 Hz, 1H), 7.71 (d, J=2.2 Hz, 1H), 7.61 (s, 1H), 7.35 (d, J=9.3 Hz, 1H), 7.32-7.20 (m, 4H), 7.14 (d, J=7.6 Hz, 1H), 6.94 (dd, J=9.3, 1.3 Hz, 1H), 6.47 (t, J=5.8 Hz, 1H), 4.30 (d, J=5.7 Hz, 2H), 2.39-2.33 (m, 1H), 2.10-2.04 (m, 1H), 1.92-1.83 (m, 1H), 1.49-1.35 (m, 2H), 0.92-0.85 (m, 2H), 0.66-0.61 (m, 2H).

The compound in Table 46 was prepared in a similar manner to rac-(1S*,2S*)-2-(3-chlorophenyl)-N-(5-(((6-cyclopropylimidazo[1,2-a]pyridin-2-yl)methyl)amino)pyridin-3-yl)cyclopropane-1-carboxamide.

TABLE 46

| | | Coupling Partner / |
|---|---|---|
| Example | Structure | Analytical Data |
| Example 575 | 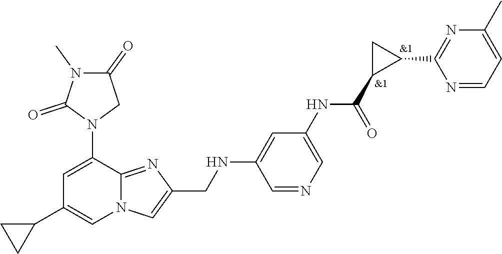 [SMILES: Cc1ccnc([C@H]2C[C@@H]2C(=O)Nc2cncc(NCc3cn4cc(C5CC5)cc(N5CC(=O)N(C)C5=O)c4n3)c2)n1] | rac-(1S*,2S*)-N-(5-aminopyridin-3-yl)-2- (4-methylpyrimidin-2-yl)cyclopropane-1- carboxamide ESI-MS [M + H]+: 552.2. ¹H NMR (400 MHz, DMSO) δ 10.25 (s, 1H), 8.50 (d, J = 5.1 Hz, 1H), 8.23 (d, J = 1.0 Hz, 1H), 7.92 (d, J = 8.2 Hz, 1H), 7.71 − 7.69 (m, 2H), 7.37 − 7.32 (m, 2H), 7.19 (d, J = 5.1 Hz, 1H), 6.50 (t, J = 5.9 Hz, 1H), 4.90 (s, 2H), 4.33 (d, J = 5.8 Hz, 2H), 2.96 (s, 3H), 2.53 − 2.49 (m, 2H), 2.40 (s, 3H), 1.95 − 1.88 (m, 1H), 1.51 − 1.44 (m, 2H), 0.94 − 0.85 (m, 2H), 0.68 − 0.61 (m, 2H). |
| | rac-(1S*,2S*)-N-(5-(((6- | |
| | cyclopropyl-8-(3-methyl-2,4- | |
| | dioxoimidazolidin-1- | |
| | yl)imidazo[1,2-a]pyridin-2- | |
| | yl)methyl)amino)pyridin-3-yl)-2- | |
| | (4-methylpyrimidin-2- | |
| | yl)cyclopropane-1-carboxamide (I- | |
| | 575) | |

### Synthesis of 1-(2-((4-chloro-2H-pyrazolo[4,3-c]pyridin-2-yl)methyl)-6-cyclopropylimidazo[1,2-a]pyridin-8-yl)-3-methylimidazolidine-2,4-dione

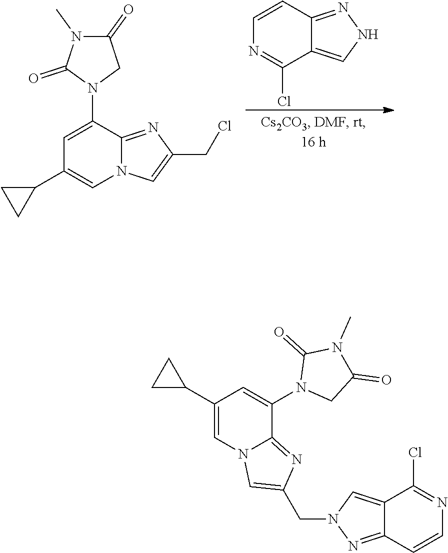 [SMILES: CN1C(=O)CN(c2cc(C3CC3)cn3cc(CCl)nc23)C1=O ; Clc1nccc2n[nH]cc12 ; CN1C(=O)CN(c2cc(C3CC3)cn3cc(Cn4cc5c(Cl)nccc5n4)nc23)C1=O]

A mixture of 1-(2-(chloromethyl)-6-cyclopropylimidazo[1,2-a]pyridin-8-yl)-3-methylimidazolidine-2,4-dione (420 mg, 1.32 mmol), 4-chloro-2H-pyrazolo[4,3-c]pyridine (200 mg, 1.32 mmol) and Cs₂CO₃ (1.3 g, 3.96 mmol) in DMF (8 mL) was stirred at 25° C. for 16 h. Water (50 mL) was added, then the reaction was extracted with EtOAc (3×100 mL). The combined organic layers were washed with brine (30 mL), dried over anhydrous Na₂SO₄, and concentrated in vacuo to give the crude, which was purified by preparative HPLC to give 1-(2-((4-chloro-2H-pyrazolo[4,3-c]pyridin-2-yl)methyl)-6-cyclopropylimidazo[1,2-a]pyridin-8-yl)-3-methylimidazolidine-2,4-dione (90 mg, 16% ) as a yellow solid. ESI-MS [M+H]+: 436.1. The compounds in Table 47 were prepared in a similar manner to 1-(2-((4-chloro-2H-pyrazolo[4,3-c]pyridin-2-yl)methyl)-6-cyclopropylimidazo[1,2-a]pyridin-8-yl)-3-methylimidazolidine-2,4-dione.

TABLE 47

| | Analytical |
|---|---|
| Intermediate | Data |
| 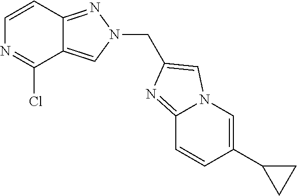 [SMILES: Clc1nccc2nn(Cc3cn4cc(C5CC5)ccc4n3)cc12] | ESI-MS [M + H]+: 324.2 |
| 4-chloro-2-((6-cyclopropylimidazo[1,2-a]pyridin- | |
| 2-yl)methyl)-2H-pyrazolo[4,3-c]pyridine | |
| 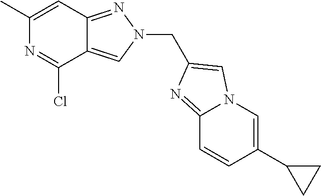 [SMILES: Cc1cc2nn(Cc3cn4cc(C5CC5)ccc4n3)cc2c(Cl)n1] | ESI-MS [M + H]+: 338.2 |
| 4-chloro-2-((6-cyclopropylimidazo[1,2-a]pyridin- | |
| 2-yl)methyl)-6-methyl-2H-pyrazolo[4,3-c]pyridine | |
| 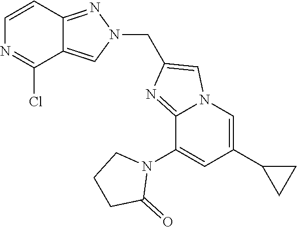 [SMILES: O=C1CCCN1c1cc(C2CC2)cn2cc(Cn3cc4c(Cl)nccc4n3)nc12] | ESI-MS [M + H]+: 407.1 |
| 1-(2-((4-chloro-2H-pyrazolo[4,3-c]pyridin-2- | |
| yl)methyl)-6-cyclopropylimidazo | |
| [1,2-a]pyridin-8-yl)pyrrolidin-2-one | |
| 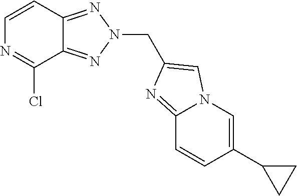 [SMILES: Clc1nccc2nn(Cc3cn4cc(C5CC5)ccc4n3)nc12] | ESI-MS [M + H]+: 325.2 |
| 4-chloro-2-((6-cyclopropylimidazo[1,2-a]pyridin- | |
| 2-yl)methyl)-2H-[1,2,3]triazolo[4,5-c]pyridine | |
| 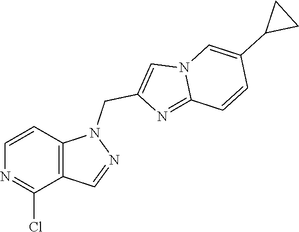 [SMILES: Clc1nccc2c1cnn2Cc1cn2cc(C3CC3)ccc2n1] | ESI-MS [M + H]+: 324.1 |
| 4-chloro-1-((6-cyclopropylimidazo[1,2-a]pyridin- | |
| 2-yl)methyl)-1H-pyrazolo[4,3-c ]pyridine | |

### Example 576

### Synthesis of rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[4,3-c]pyridin-4-yl)-2-(4-methylpyrimidin-2-yl)cyclopropane-1-carboxamide (I-576)

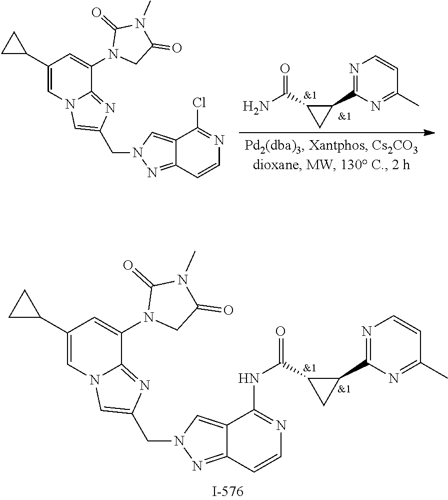 [SMILES: CN1C(=O)CN(c2cc(C3CC3)cn3cc(Cn4cc5c(Cl)nccc5n4)nc23)C1=O ; Cc1ccnc([C@H]2C[C@@H]2C(N)=O)n1 ; Cc1ccnc([C@H]2C[C@@H]2C(=O)Nc2nccc3nn(Cc4cn5cc(C6CC6)cc(N6CC(=O)N(C)C6=O)c5n4)cc23)n1]

To a mixture of 1-(2-((4-chloro-2H-pyrazolo[4,3-c]pyridin-2-yl)methyl)-6-cyclopropylimidazo[1,2-a]pyridin-8-yl)-3-methylimidazolidine-2,4-dione (25 mg, 0.05 mmol), rac-(1S*,2S*)-2-(4-methylpyrimidin-2-yl)cyclopropane-1-carboxamide (10.2 mg, 0.05 mmol), and Cs₂CO₃ (56 mg, 0.15 mmol) in dioxane (3 mL) were added Pd₂(dba)₃ (15.7 mg, 0.015 mmol) and xantphos (20 mg, 0.03 mmol). The mixture was stirred at 90° C. for 2 h under N₂, then cooled to room temperature. The reaction mixture was filtered through Celite and the filter cake was washed with DCM/MeOH (10/1, 50 mL). The filtrate was concentrated in vacuo to give the crude, which was purified by preparative TLC (eluent: DCM/MeOH=10/1) to give (1S*,2S*)—N-(2-((6 cyclopropyl-8-(3-methyl-2,4-dioxoimidazolidin-1-yl)imidazo[1,2-a]pyridin-2-yl)methyl)-2H-pyrazolo[4,3-c]pyridin-4-yl)-2-(4-methylpyrimidin-2-yl)cyclopropane-1-carboxamide (5 mg, 330) as a white solid. ESI-MS [M+H]+: 577.2. ¹H NMR (400 MHz, DMSO) δ 11.04 (s, 1H), 8.85 (s, 1H), 8.54 (d, J=5.1, 1H), 8.46 (s, 1H), 8.29 (s, 1H), 7.89 (s, 1H), 7.86 (d, J=5.8, 1H), 7.39 (d, J=1.4, 1H), 7.25 (d, J=5.9, 1H), 7.22 (d, J=5.1, 1H), 5.77 (s, 2H), 4.88 (s, 2H), 2.96 (s, 3H), 2.76-2.65 (m, 2H), 2.43 (s, 3H), 1.98-1.93 (m, 1H), 1.63-1.57 (m, 2H), 0.98-0.92 (m, 2H), 0.67-0.63 (d, 2H). The compounds in Table 48 were prepared in a similar manner to (1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[4,3-c]pyridin-4-yl)-2-(4-methylpyrimidin-2-yl)cyclopropane-1-carboxamide using the indicated coupling partner.

TABLE 48

| | | Coupling Partner / |
|---|---|---|
| Example | Structure | Analytical Data |
| Example 577 | 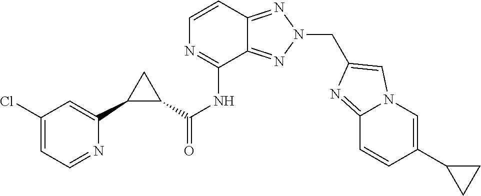 [SMILES: O=C(Nc1nccc2nn(Cc3cn4cc(C5CC5)ccc4n3)nc12)[C@H]1C[C@@H]1c1cc(Cl)ccn1] | 4-chloro-2-((6-cyclopropylimidazo[1,2- a]pyridin-2-yl)methyl)-2H-pyrazolo[4,3- c]pyridine ESI-MS [M + H]+: 484.1. ¹H NMR (400 MHz, DMSO) δ 11.02 (s, 1H), 8.78 (s, 1H), 8.44 (d, J = 5.4, 1H), 8.35 (s, 1H), 7.89 − 7.83 (m, 1H), 7.83 (s, 1H), 7.67 (d, J = 1.8, 1H), 7.41-7.38 (m, 1H), 7.37-7.35 (m, 1H), 7.27-7.20 (m, 1H), 7.02-6.98 (m, 1H), 5.74 (s, 2H), 2.70- 2.66 (m, 2H), 1.94-1.89 (m, 1H), 1.60-1.54 (m, 2H), 0.94 − 0.89 (m, 2H), 0.69 − 0.65 (m, 2H). |
| | (1S,2S)-2-(4-chloropyridin-2-yl)-N- | |
| | (2-((6-cyclopropylimidazo[1,2- | |
| | a]pyridin-2-yl)methyl)-2H- | |
| | pyrazolo[4,3-c]pyridin-4- | |
| | yl)cyclopropane-1-carboxamide (I- | |
| | 577) | |
| Example 578 | 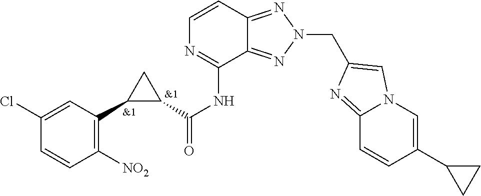 [SMILES: O=C(Nc1nccc2nn(Cc3cn4cc(C5CC5)ccc4n3)nc12)[C@H]1C[C@@H]1c1cc(Cl)ccc1[N+](=O)[O-]] | 4-chloro-2-((6-cyclopropylimidazo[1,2- a]pyridin-2-yl)methyl)-2H-pyrazolo[4,3- c]pyridine ESI-MS [M + H]+: 528.2, ¹H NMR (400 MHz, DMSO) δ 11.02 (s, 1H), 8.82 (s, 1H), 8.35 (s, 1H), 8.05 (d, J = 9.2 Hz, 1H), 7.91 − 7.80 (m, 2H), 7.62- 7.60 (m, 2H), 7.39 (d, J = 9.3 Hz, 1H), 7.25 (d, J = 6.1 Hz, 1H), 7.01 − 6.98 (m, 1H), 5.75 (s, 2H), 2.81 − 2.76 (m, 1H), 2.51 − 2.44 (m, 1H), 1.97 − 1.87 (m, 1H), 1.63 − 1.50 (m, 2H), 0.96 − 0.82 (m, 2H), 0.69 − 0.65 (m, 2H). |
| | rac-(1S*,2S*)-2-(5-chloro-2- | |
| | nitrophenyl)-N-(2-((6- | |
| | cyclopropylimidazo[1,2-a]pyridin-2- | |
| | yl)methyl)-2H-pyrazolo[4,3- | |
| | c]pyridin-4-yl)cyclopropane-1- | |
| | carboxamide (I-578) | |
| Example 579 | 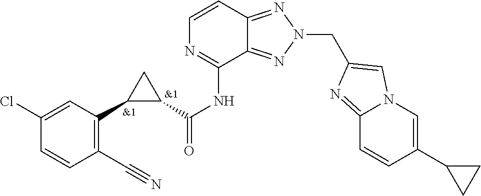 [SMILES: N#Cc1ccc(Cl)cc1[C@H]1C[C@@H]1C(=O)Nc1nccc2nn(Cc3cn4cc(C5CC5)ccc4n3)nc12] | 4-chloro-2-((6-cyclopropylimidazo[1,2- a]pyridin-2-yl)methyl)-2H-pyrazolo[4,3- c]pyridine ESI-MS [M + H]⁺: 508.2. ¹H NMR (400 MHz, DMSO) δ 11.04 (s, 1H), 8.81 (s, 1H), 8.34 (s, 1H), 7.88 − 7.82 (m, 3H), 7.53 (dd, J = 8.3, 1.9 Hz, 1H), 7.45 (s, 1H), 7.39 (d, J = 9.3 Hz, 1H), 7.25 (d, J = 6.2 Hz, 1H), 7.00 (dd, J = 9.4, 1.8 Hz, 1H), 5.75 (s, 2H), 2.70 − 2.65 (m, 2H), 1.95 − 1.88 (m, 1H), 1.66 − 1.62 (m, 2H), 0.94 − 0.89 (m, 2H), 0.69 − 0.65 (m, 2H). |
| | rac-(1S*,2S*)-2-(5-chloro-2- | |
| | cyanophenyl)-N-(2-((6- | |
| | cyclopropylimidazo[1,2-a]pyridin-2- | |
| | yl)methyl)-2H-pyrazolo[4,3- | |
| | c]pyridin-4-yl)cyclopropane-1- | |
| | carboxamide (I-579) | |
| Example 580 | 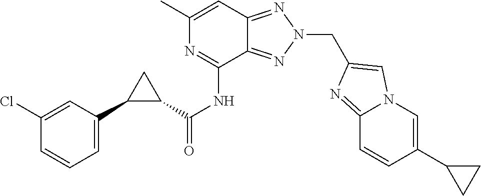 | 4-chloro-2-((6-cyclopropylimidazo[1,2- a]pyridin-2-yl)methyl)-2H-pyrazolo[4,3- c]pyridine ESI-MS [M + H]⁺: 483.1. ¹H NMR (400 MHz, DMSO) δ 10.98 (s, 1H), 8.80 (s, 1H), 8.35 (s, 1H), 7.86 − 7.83 (m, 2H), 7.41 − 7.17 (m, 6H), 7.00 (dd, J = 9.4, 1.7 Hz, 1H), 5.74 (s, 2H), 2.52 − 2.51 (m, 1H), 2.47 − 2.44 (m, 1H), 1.92 − 1.89 (m, 1H), 1.58 − 1.44 (m, 2H), 0.94 − 0.89 (m, 2H), 0.69 − 0.65 (m, 2H). |
| | (1S,2S)-2-(3-chlorophenyl)-N-(2-((6- | |
| | cyclopropylimidazo[1,2-a]pyridin-2- | |
| | yl)methyl)-2H-pyrazolo[4,3- | |
| | c]pyridin-4-yl)cyclopropane-1- | |
| | carboxamide (I-580) | |
| Example 581 | 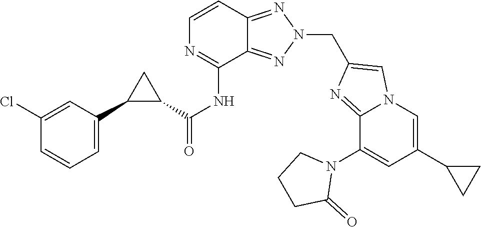 [SMILES: O=C(Nc1nccc2nn(Cc3cn4cc(C5CC5)cc(N5CCCC5=O)c4n3)nc12)[C@H]1C[C@@H]1c1cccc(Cl)c1] | 4-chloro-2-((6-cyclopropylimidazo[1,2- a]pyridin-2-yl)methyl)-6-methyl-2H- pyrazolo[4,3-c]pyridine ESI-MS [M + H]⁺: 497.2. ¹H NMR (400 MHz, DMSO) δ 10.98 (s, 1H), 8.73 (s, 1H), 8.35 (s, 1H), 7.80 (s, 1H), 7.40 − 7.28 (m, 4H), 7.19 − 7.16 (m, 1H), 7.04 − 6.98 (m, 2H), 5.69 (s, 2H), 2.49 − 2.44 (m, 2H), 2.38 (s, 3H), 1.95 − 1.90 (m, 1H), 1.56 − 1.51 (m, 1H), 1.46 − 1.41 (m, 1H), 0.94 − 0.89 (m, 2H), 0.69 − 0.65 (m, 2H). |
| | (1S,2S)-2-(3-chlorophenyl)-N-(2-((6- | |
| | cyclopropylimidazo[1,2-a]pyridin-2- | |
| | yl)methyl)-6-methyl-2H- | |
| | pyrazolo[4,3-c]pyridin-4- | |
| | yl)cyclopropane-1-carboxamide (I- | |
| | 581) | |
| Example 582 | 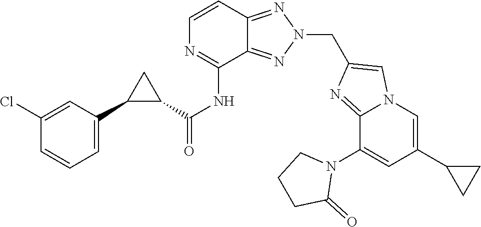 [SMILES: O=C(Nc1nccc2nn(Cc3cn4cc(C5CC5)cc(N5CCCC5=O)c4n3)nc12)[C@H]1C[C@@H]1c1cccc(Cl)c1] | 1-(2-((4-chloro-2H-pyrazolo[4,3-c]pyridin- 2-yl)methyl)-6-cyclopropylimidazo[1,2- a]pyridin-8-yl)pyrrolidin-2-one ESI-MS [M + H]⁺: 566.1. ¹H NMR (400 MHz, DMSO) δ 10.98 (s, 1H), 8.86 (s, 1H), 8.28 (d, J = 1.2 Hz, 1H), 7.867 − 7.85 (m, 2H), 7.35 − 7.17 (m, 6H), 5.76 (s, 2H), 4.14 (t, J = 7.1 Hz, 2H), 2.47 − 2.42 (m, 4H), 2.15 − 2.08 (m, 2H), 1.96 − 1.90 (m, 1H), 1.56 − 1.44 (m, 2H), 0.950.91 (m, 2H), 0.67 − 0.63 (m, 2H). |
| | (1S,2S)-N-(5-((6-cyclopropyl-8-(3- | |
| | methyl-2,4-dioxoimidazolidin-1- | |
| | yl)imidazo[1,2-a]pyridin-2- | |
| | yl)methoxy)pyridazin-3-yl)-2-(4- | |
| | methylpyrimidin-2-yl)cyclopropane- | |
| | 1-carboxamide (I-582) | |
| Example 583 | 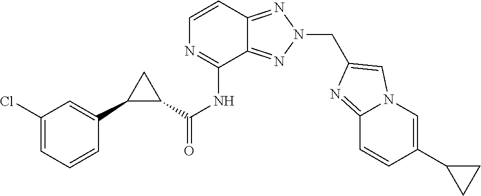 [SMILES: O=C(Nc1nccc2nn(Cc3cn4cc(C5CC5)ccc4n3)nc12)[C@H]1C[C@@H]1c1cccc(Cl)c1] | 4-chloro-2-((6-cyclopropylimidazo[1,2- a]pyridin-2-yl)methyl)-2H- [1,2,3]triazolo[4,5-c]pyridine ESI-MS (M + H)+: 484.1, ¹H NMR (400 MHz, DMSO) δ 11.04 (s, 1H), 8.40 (s, 1H), 8.33 (s, 1H), 8.10 (s, 1H), 7.89 (s, 1H), 7.61 (s, 1H), 7.35 − 7.15 (m, 5H), 6.98 (d, J = 9.4 Hz, 1H), 6.07 (s, 2H), 2.44 − 2.36 (m, 2H), 1.95 − 1.86 (m, 1H), 1.57 − 1.48 (m, 1H), 1.44 − 1.40 (m, 1H), 0.95 − 0.85 (m, 2H), 0.69 − 0.60 (m, 2H). |
| | (1S,2S)-2-(3-chlorophenyl)-N-(2-((6- | |
| | cyclopropylimidazo[1,2-a]pyridin-2- | |
| | yl)methyl)-2H-[1,2,3]triazolo[4,5- | |
| | c]pyridin-4-yl)cyclopropane-1- | |
| | carboxamide (I-583) | |
| Example 584 | 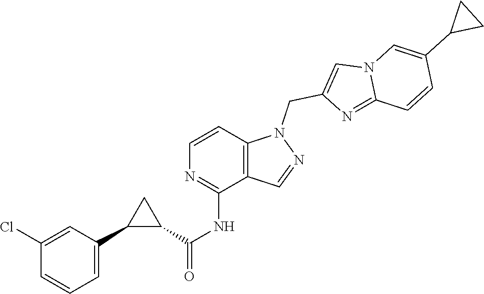 [SMILES: O=C(Nc1nccc2c1cnn2Cc1cn2cc(C3CC3)ccc2n1)[C@H]1C[C@@H]1c1cccc(Cl)c1] | 4-chloro-1-((6-cyclopropylimidazo[1,2- a]pyridin-2-yl)methyl)-1H-pyrazolo[4,3- c]pyridine ESI-MS [M + H]⁺: 483.1. ¹H NMR (400 MHz, DMSO) δ 10.99 (s, 1H), 8.31 (d, J = 0.8 Hz, 1H), 8.26 − 8.26 (m, 1H), 8.04 (d, J = 6.0 Hz, 1H), 7.62 (s, 1H), 7.50-7.48 (m, 1H), 7.34 − 7.23 (m, 4H), 7.18 − 7.16 (m, 1H), 6.96-6.93 (m, 1H), 5.68 (s, 2H), 1.99 − 1.99 (m, 1H), 1.91 − 1.83 (m, 1H), 1.57 − 1.53 (m, 1H), 1.47 − 1.42 (m, 2H), 0.90 − 0.85 (m, 2H), 0.64 − 0.60 (m, 2H). |
| | (1S,2S)-2-(3-chlorophenyl)-N-(1-((6- | |
| | cyclopropylimidazo[1,2-a]pyridin-2- | |
| | yl)methyl)-1H-pyrazolo[4,3- | |
| | c]pyridin-4-yl)cyclopropane-1- | |
| | carboxamide (I-584) | |

### Examples 585-586

### Synthesis of rac-(1S*,2S*)-2-(5-chloro-2-(1H-tetrazol-1-yl)phenyl)-N-(2-((6-cyclopropylimidazo imidazo[1,2-a]pyridin-2-yl)methyl)-2H-pyrazolo[4,3-c]pyridin-4-yl)cyclopropane-1-carboxamide

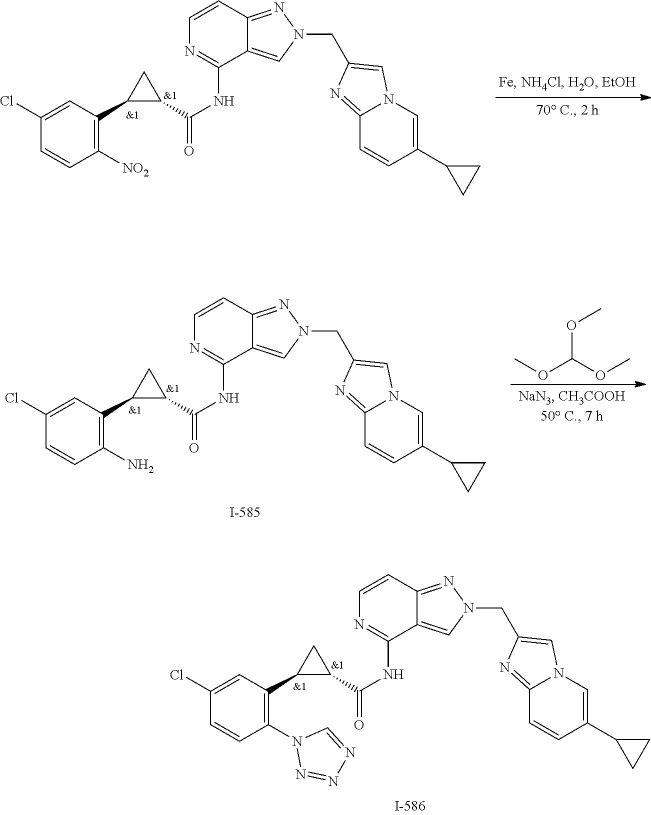

### Synthesis of rac-(1S*,2S*)-2-(2-amino-5-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide (I-585)

The title compound was prepared in a similar manner to rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide from rac-(1S*,2S*)-2-(5-chloro-2-nitrophenyl)-N-(2-((6-cyclopropylimidazo[1,2-a]pyridin-2-yl)methyl)-2H-pyrazolo[4,3-c]pyridin-4-yl)cyclopropane-1-carboxamide. ESI-MS [M+H]⁺: 498.2; ¹H NMR (400 MHz, DMSO) δ 11.07 (s, 1H), 8.83 (s, 1H), 8.35 (s, 1H), 7.88-7.83 (m, 2H), 7.39 (d, J=9.3 Hz, 1H), 7.25 (d, J=6.0 Hz, 1H), 7.05-6.90 (m, 3H), 6.65 (d, J=8.5 Hz, 1H), 5.74 (s, 2H), 5.14 (s, 2H), 2.41-2.32 (m, 1H), 2.26-2.21 (m, 1H), 1.97-1.87 (m, 1H), 1.55-1.46 (m, 1H), 1.19-1.11 (m, 1H), 0.97-0.81 (m, 2H), 0.72-0.62 (m, 2H).

### Synthesis of rac-(1S*,2S*)-2-(5-chloro-2-(1H-tetrazol-1-yl)phenyl)-N-(2-((6-cyclopropylimidazo[1,2-a]pyridin-2-yl)methyl)-2H-pyrazolo[4,3-c]pyridin-4-yl)cyclopropane-1-carboxamide (I-586)

The title compound was prepared in a similar manner to rac-(1S*,2S*)-2-(5-chloro-2-(1H-tetrazol-1-yl)phenyl)-N-(4-(((6-cyclopropylimidazo[1,2-a]pyridin-2-yl)methyl)amino)pyridin-2-yl)cyclopropane-1-carboxamide from rac-(1S*,2S*)-2-(2-amino-5-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide. ESI-MS [M+H]⁺: 551.3, ¹H NMR (400 MHz, MeOD) δ 9.54 (s, 1H), 8.67 (s, 1H), 8.21 (s, 1H), 7.84 (s, 2H), 7.55-7.52 (m, 3H), 7.40 (d, J=9.3 Hz, 1H), 7.26 (d, J=6.1 Hz, 1H), 7.14-7.07 (m, 1H), 5.78 (s, 2H), 2.37-2.30 (m, 1H), 2.16-2.10 (m, 1H), 1.96-1.92 (m, 1H), 1.53-1.44 (m, 2H), 1.00-0.94 (m, 2H), 0.73-0.69 (m, 2H).

### Example 587

### Synthesis of rac-(1S*,2S*)-2-(3-chlorophenyl)-N-(6-((6-cyclopropylimidazo[1,2-a]pyridin-2-yl)methyl)pyrimidin-4-yl)cyclopropane-1-carboxamide (I-587)

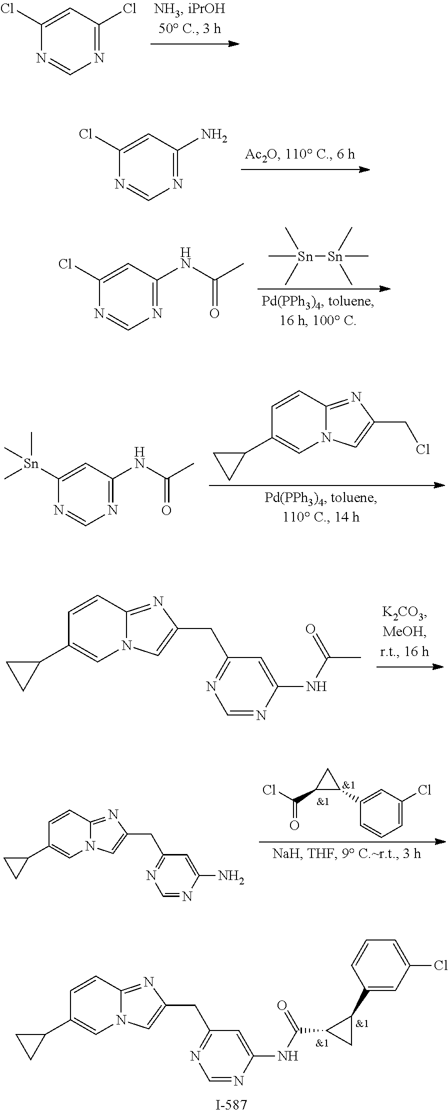

Synthesis of 6-chloropyrimidin-4-amine. A mixture of 4,6-dichloropyrimidine (3.2 g, 21 mmol) and NH₄OH (15 mL) in i-PrOH (15 mL) was stirred at 50° C. for 3 h in a sealed tube. The reaction mixture was concentrated in vacuo to give 6-chloropyrimidin-4-amine (2.8 g, quant.) as a white solid. ESI-MS [M+H]⁺: 130.1.

Synthesis of N-(6-chloropyrimidin-4-yl)acetamide. A mixture of 6-chloropyrimidin-4-amine (2.8 g, 21 mmol) in acetic anhydride (20 mL) was stirred at 110° C. for 6 h. The reaction mixture was cooled to room temperature and concentrated in vacuo to give the residue, which was diluted in EtOAc (150 mL) and washed with saturated aq·NaHCO₃ (60 mL). The organic layer was washed with brine (60 mL), dried over Na₂SO₄, and concentrated in vacuo to give the crude product, which was purified by silica gel column chromatography (eluent: PE/EtOAc=2/1) to give N-(6-chloropyrimidin-4-yl)acetamide (2.9 g, 79%) as a yellow solid. ESI-MS [M+H]+: 172.0.

Synthesis of N-(6-(trimethylstannyl)pyrimidin-4-yl)acetamide. A mixture of N-(6-chloropyrimidin-4-yl)acetamide (2.0 g, 12 mmol), 1,1,1,2,2,2-hexamethyldistannane (7.6 g, 23 mmol) and Pd(PPh₃)₄(1.4 g, 1.2 mmol) in toluene (100 mL) was stirred at 100° C. for 6 h. After cooling to room temperature, the reaction mixture was filtered and the filtrate was concentrated in vacuo to give the crude product, which was purified by column chromatography on neutral Al₂O₃ (eluent: PE/EtOAc=2/1) to give N-(6-(trimethylstannyl)pyrimidin-4-yl)acetamide (3.2 g, 91%) as a white solid. ESI-MS [M+H]⁺: 302.0.

Synthesis of N-(6-((6-cyclopropylimidazo[1,2-a]pyridin-2-yl)methyl)pyrimidin-4-yl)acetamide. A mixture of N-(6-(trimethylstannyl)pyrimidin-4-yl)acetamide (2.4 g, 8.0 mmol), 2-(chloromethyl)-6-cyclopropylimidazo[1,2-a]pyridine (1.7 g, 8.0 mmol) and Pd(PPh₃)₄ (0.92 g, 0.80 mmol) in toluene (100 mL) was stirred at 110° C. for 14 h. The reaction mixture was cooled to room temperature and concentrated in vacuo to give the crude product, which was purified by column chromatography on silica gel column chromatography (eluent: DCM/MeOH=20/1) to give N-(6-((6-cyclopropylimidazo[1,2-a]pyridin-2-yl)methyl)pyrimidin-4-yl)acetamide (0.20 g, 8%) as a yellow solid. ESI-MS [M+H]⁺: 308.2.

Synthesis of 6-((6-cyclopropylimidazo[1,2-a]pyridin-2-yl)methyl)pyrimidin-4-amine. To a stirred solution of N-(6-((6-cyclopropylimidazo[1,2-a]pyridin-2-yl)methyl)pyrimidin-4-yl)acetamide (0.20 g, 0.65 mmol) in MeOH (6.0 mL) was added K₂CO₃ (0.18 g, 1.3 mmol) and the mixture was stirred at room temperature for 16 h. The reaction mixture was concentrated in vacuo to give the crude, which was diluted in water (15 mL), extracted with DCM/MeOH (10/1, 4×30 mL). The combined organics was washed with brine (30 mL), dried over Na₂SO₄ and concentrated in vacuo to give the crude product, which was purified by silica gel column chromatography (eluent: DCM/MeOH=15/1) to afford 6-((6-cyclopropylimidazo[1,2-a]pyridin-2-yl)methyl)pyrimidin-4-amine (120 mg, 70%) as a yellow solid. ESI-MS [M+H]⁺: 266.2.

Synthesis of rac-(1S*,2S*)-2-(3-chlorophenyl)-N-(6-((6-cyclopropylimidazo[1,2-a]pyridin-2-yl)methyl)pyrimidin-4-yl)cyclopropane-1-carboxamide (I-587). To a stirred solution of 6-((6-cyclopropylimidazo[1,2-a]pyridin-2-yl)methyl)pyrimidin-4-amine (70 mg, 0.26 mmol) in THF (4.0 mL) was added NaH (21 mg, 0.53 mmol, 60% dispersion in mineral oil) in one portion at 0° C. After 10 min, a solution of rac-(1S*,2S*)-2-(3-chlorophenyl)cyclopropane-1-carbonyl chloride (110 mg, 0.53 mmol) in THF (2 mL) was added dropwise. After stirring at room temperature for 3 h, the reaction mixture was quenched with water (20 mL) and extracted with EtOAc (2×50 mL). The combined organics was washed with brine (40 mL), dried over Na₂SO₄, and concentrated in vacuo to give the crude product, which was purified by preparative HPLC to give rac-(1S*,2S*)-2-(3-chlorophenyl)-N-(6-((6-cyclopropylimidazo[1,2-a]pyridin-2-yl)methyl)pyrimidin-4-yl)cyclopropane-1-carboxamide (7.0 mg, 6%) as a white solid.

ESI-MS [M+H]⁺: 444.1 ¹H NMR (400 MHz, DMSO) δ 11.12 (s, 1H), 8.76 (s, 1H), 8.32 (s, 1H), 7.98 (s, 1H), 7.67 (s, 1H), 7.35-7.25 (m, 4H), 7.14 (d, J=7.6 Hz, 1H), 6.96 (d, J=9.3 Hz, 1H), 4.10 (s, 2H), 2.44-2.33 (m, 2H), 1.96-1.89 (m, 1H), 1.52-1.41 (m, 2H), 0.97-0.86 (m, 2H), 0.70-0.66 (m, 2H).

### Example 588

### Synthesis of rac-(1S*,2S*)-2-(3-chlorophenyl)-N-((1-((6-cyclopropylimidazo[1,2-a]pyridin-2-yl)methyl)-1H-1,2,3-triazol-4-yl methylcyclopropane-1-carboxamide (I-588)

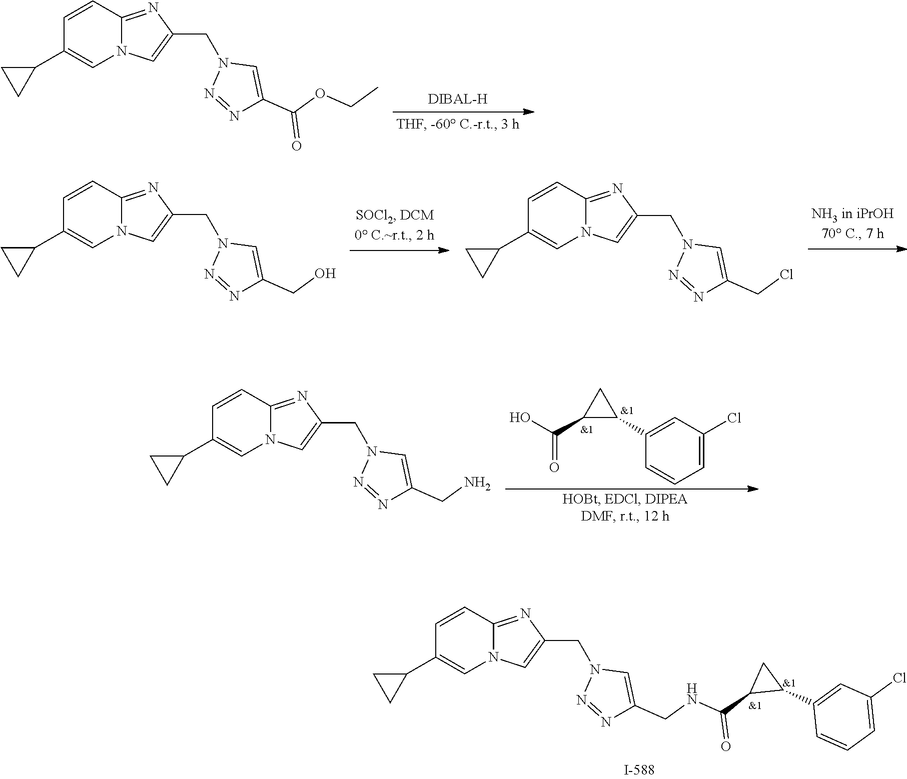 [SMILES: CCOC(=O)c1cn(Cc2cn3cc(C4CC4)ccc3n2)nn1 ; OCc1cn(Cc2cn3cc(C4CC4)ccc3n2)nn1 ; ClCc1cn(Cc2cn3cc(C4CC4)ccc3n2)nn1 ; NCc1cn(Cc2cn3cc(C4CC4)ccc3n2)nn1 ; O=C(O)[C@H]1C[C@@H]1c1cccc(Cl)c1 ; O=C(NCc1cn(Cc2cn3cc(C4CC4)ccc3n2)nn1)[C@H]1C[C@@H]1c1cccc(Cl)c1]

Synthesis of (1-((6-cyclopropylimidazo[1,2-a]pyridin-2-yl)methyl)-1H-1,2,3-triazol-4-yl)methanol. To a mixture of ethyl 1-((6-cyclopropylimidazo[1,2-a]pyridin-2-yl)methyl)-1H-1,2,3-triazole-4-carboxylate (815 mg, 2.62 mmol) in THF (10 mL) was added DIBALH (5.3 mL, 5.3 mmol, 1M in THF) at −60° C. The mixture was warmed to room temperature and stirred for 3 h. The mixture was quenched with NH₄Cl (sat·aq·, 20 mL), filtered through Celite®, and the filter cake was washed with EtOAc (30 mL×3). The aqueous filtrate was extracted with EtOAc (20 mL×3). The combined organic layers were washed with brine (50 mL), dried over Na₂SO₄, and concentrated in vacuo. The residue was purified by column chromatography (eluent: EtOAc/PE from 0 to 30%) to afford (1-((6-cyclopropylimidazo[1,2-a]pyridin-2-yl)methyl)-1H-1,2,3-triazol-4-yl)methanol (425 mg, 60% yield) as a white solid. ESI-MS [M+H]+: 270.1

Synthesis of 2-((4-(chloromethyl)-1H-1,2,3-triazol-1-yl)methyl)-6-cyclopropylimidazo[1,2-a]pyridine. To a solution of (1-((6-cyclopropylimidazo[1,2-a]pyridin-2-yl)methyl)-1H-1,2,3-triazol-4-yl)methanol (300 mg, 1.1 mmol) in DCM (20 mL) was added SOCl₂ (2 mL) at 0° C. The resulting reaction was stirred at room temperature for 2 h and concentrated in vacuo to give the crude (320 mg crude), which was used into next step without further purification. ESI-MS [M+H]+: 288.1.

Synthesis of (1-((6-cyclopropylimidazo[1,2-a]pyridin-2-yl)methyl)-1H-1,2,3-triazol-4-yl)methanamine. A solution of 2-((4-(chloromethyl)-1H-1,2,3-triazol-1-yl)methyl)-6-cyclopropylimidazo[1,2-a]pyridine (320 mg crude from previous step) in NH₃/iPrOH (7M, 70 mL) was stirred in a sealed tube at 70° C. for 7 h. The reaction was cooled to room temperature and concentrated in vacuo to give the crude, which was purified with silica gel column chromatography (eluent: DCM/MeOH=10/1) to give (1-((6-cyclopropylimidazo[1,2-a]pyridin-2-yl)methyl)-1H-1,2,3-triazol-4-yl)methanamine (130 mg, 44% over 2 steps). ESI-MS [M+H]+: 269.2.

Synthesis of rac-(1S*,2S*)-2-(3-chlorophenyl)-N-((1-((6-cyclopropylimidazo[1,2-a]pyridin-2-yl)methyl)-1H-1,2,3-triazol-4-yl)methyl)cyclopropane-1-carboxamide (I-588). A mixture of (1-((6-cyclopropylimidazo[1,2-a]pyridin-2-yl)methyl)-1H-1,2,3-triazol-4-yl)methanamine (100 mg, 0.73 mmol), rac-(1S*,2S*)-2-(3-chlorophenyl)cyclopropane-1-carboxylic acid (143 mg, 0.73 mmol), HOBt (148.5 mg, 1.1 mmol), EDCI (211 mg, 1.1 mmol), and DIPEA (282.5 mg, 2.19 mmol) in DMF (5 mL) was stirred at room temperature for 12 h. The resulting mixture was diluted with water (30 mL) and extracted with EtOAc (3×30 mL). The combined organic layers were washed with brine (30 mL), dried over Na₂SO₄, and concentrated in vacuo to give the crude, which was purified with preparative TLC (eluent: DCM/MeOH=10/1) to give rac-(1S*,2S*)-2-(3-chlorophenyl)-N-((1-((6-cyclopropylimidazo[1,2-a]pyridin-2-yl)methyl)-1H-1,2,3-triazol-4-yl)methyl)cyclopropane-1-carboxamide (25 mg, 8%) as a white solid. ESI-MS [M+H]+: 447.2. ¹H NMR (400 MHz, DMSO) δ 8.59 (t, J=5.0 Hz, 1H), 8.35 (s, 1H), 7.92 (s, 1H), 7.82 (s, 1H), 7.40 (d, J=9.3 Hz, 1H), 7.28 (t, J=7.7 Hz, 1H), 7.22 (d, J=8.0 Hz, 1H), 7.18 (s, 1H), 7.09 (d, J=7.5 Hz, 1H), 7.01 (d, J=9.4 Hz, 1H), 5.63 (s, 2H), 4.32 (d, J=5.4 Hz, 2H), 2.31-2.24 (m, 1H), 1.95-1.88 (m, 2H), 1.39-1.31 (m, 1H), 1.25-1.23 (m, 1H), 0.96-0.88 (m, 2H), 0.71-0.64 (m, 2H).

### Examples 589-590

### Synthesis of rac-(1S*,2S*)-2-(3-chlorophenyl)-N-(1-(1-((6-cyclopropylimidazo[1,2-a]pyridin-2-yl)methyl)-1H-1,2,3-triazol-4-yl)ethyl)cyclopropane-1-carboxamide

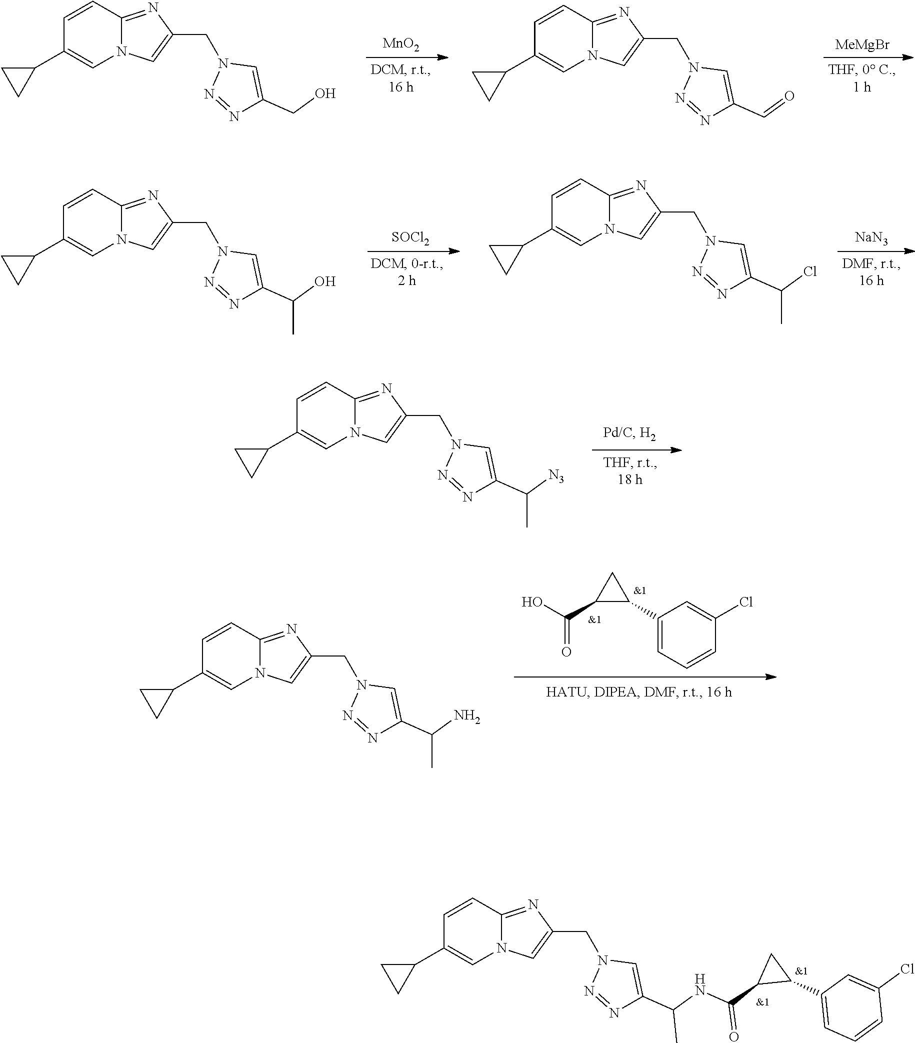

Synthesis of 1-((6-cyclopropylimidazo[1,2-a]pyridin-2-yl)methyl)-1H-1,2,3-triazole-4-carbaldehyde. A mixture of (1-((6-cyclopropylimidazo[1,2-a]pyridin-2-yl)methyl)-1H-1,2,3-triazol-4-yl)methanol (3.0 g, 11.1 mmol) and MnO₂ (9.7 g, 111 mmol) in DCM (50 mL) was stirred at room temperature for 16 h. The reaction mixture was filtered through Celite® and the filter cake was washed with DCM/MeOH (10/1, 100 mL). The filtrate was concentrated in vacuo to give the crude, which was purified by column chromatography (eluent: EtOAc/PE from 0 to 30%) to afford 1-((6-cyclopropylimidazo[1,2-a]pyridin-2-yl)methyl)-1H-1,2,3-triazole-4-carbaldehyde (2.2 g, 73% yield) as a white solid. ESI-MS [M+H]+: 268.2

Synthesis of 1-(1-((6-cyclopropylimidazo[1,2-a]pyridin-2-yl)methyl)-1H-1,2,3-triazol-4-yl)ethan-1-ol. To a mixture of 1-((6-cyclopropylimidazo[1,2-a]pyridin-2-yl)methyl)-1H-1,2,3-triazole-4-carbaldehyde (2.0 g, 7.5 mmol) in THF (100 mL) was added methylmagnesium bromide (5.0 mL, 15.0 mmol, 3M in THF) at 0° C., and the mixture was stirred at 0° C. for 1 h. The mixture was quenched with NH₄Cl (sat·aq·, 100 mL) and extracted with EtOAc (50 mL×3). The combined organic layers were washed with brine (50 mL), dried over Na₂SO₄, and concentrated. The residue was purified by column chromatography (eluent: EtOAc/PE from 0 to 50%) to afford 1-(1-((6-cyclopropylimidazo[1,2-a]pyridin-2-yl)methyl)-1H-1,2,3-triazol-4-yl)ethan-1-ol (1.5 g, 71% yield) as a white solid. ESI-MS [M+H]+: 284.1

Synthesis of 2-((4-(1-chloroethyl)-1H-1,2,3-triazol-1-yl)methyl)-6-cyclopropylimidazo[1,2-a]pyridine. To a mixture of 1-(1-((6-cyclopropylimidazo[1,2-a]pyridin-2-yl)methyl)-1H-1,2,3-triazol-4-yl)ethan-1-ol (500 mg, 1.77 mmol) in DCM (10 mL) was added SOCl₂ (1 mL) at 0° C. and the mixture was stirred at room temperature for 2 h. The mixture was concentrated in vacuo to afford 2-((4-(1-chloroethyl)-1H-1,2,3-triazol-1-yl)methyl)-6-cyclopropylimidazo[1,2-a]pyridine (600 mg, crude) as a yellow oil which was used in the next step. ESI-MS [M+H]+: 302.1

Synthesis of 2-((4-(1-azidoethyl)-1H-1,2,3-triazol-1-yl)methyl)-6-cyclopropylimidazo[1,2-a]pyridine. A mixture of 2-((4-(1-chloroethyl)-1H-1,2,3-triazol-1-yl)methyl)-6-cyclopropylimidazo[1,2-a]pyridine (600 mg, 1.77 mmol), NaN₃ (230 mg, 3.54 mmol) in DMF (10 mL) was stirred at room temperature for 16 h. The mixture was quenched with water (100 mL) and extracted with EtOAc (30 mL×3). The combined organic layers were washed with brine (30 mL), dried over Na₂SO₄ and concentrated in vacuo. The residue was purified by flash column chromatography (eluent: EtOAc/PE from 0 to 10%) to afford 2-((4-(1-azidoethyl)-1H-1,2,3-triazol-1-yl)methyl)-6-cyclopropylimidazo[1,2-a]pyridine (500 mg, 92% yield, for 2 steps) as a yellow oil. ESI-MS [M+H]+: 309.1

Synthesis of 1-(1-((6-cyclopropylimidazo[1,2-a]pyridin-2-yl)methyl)-1H-1,2,3-triazol-4-yl)ethan-1-amine. A mixture of 2-((4-(1-azidoethyl)-1H-1,2,3-triazol-1-yl)methyl)-6-cyclopropylimidazo[1,2-a]pyridine (250 mg, 0.81 mmol) and Pd/C (50 mg) in THF (10 mL) was stirred at room temperature for 18 h. The mixture was filtered and the filtrate was concentrated in vacuo to give 1-(1-((6-cyclopropylimidazo[1,2-a]pyridin-2-yl)methyl)-1H-1,2,3-triazol-4-yl)ethan-1-amine (150 mg, crude) as a brown oil which was used in the next step directly. ESI-MS [M+H]+: 283.2

Synthesis of rac-(1S*,2S*)-2-(3-chlorophenyl)-N-(1-(1-((6-cyclopropylimidazo[1,2-a]pyridin-2-yl)methyl)-1H-1,2,3-triazol-4-yl)ethyl)cyclopropane-1-carboxamide. To a mixture of 1-(1-((6-cyclopropylimidazo[1,2-a]pyridin-2-yl)methyl)-1H-1,2,3-triazol-4-yl)ethan-1-amine (60 mg, 0.21 mmol), rac-(1S*,2S*)-2-(3-chlorophenyl)cyclopropane-1-carboxylic acid (42 mg, 0.21 mmol), DIPEA (54 mg, 0.42 mmol) in DMF (5 mL) was added HATU (96 mg, 0.25 mmol) and the mixture was stirred at room temperature for 18 h. The mixture was quenched with water (50 mL) and extracted with EtOAc (30 mL×3). The combined organic layers were washed with brine (30 mL), dried over Na₂SO₄ and concentrated in vacuo. The residue was purified by preparative TLC (eluent: EtOAc/PE=25%) to afford isomer 1 (12 mg, 12% yield) and isomer 2 (13 mg, 13% yield) as white solids. ESI-MS [M+H]+: 461.2

Isomer 1 (I-589): ESI-MS [M+H]+: 461.2. ¹H NMR (400 MHz, DMSO) δ 8.53 (d, J=8.1 Hz, 1H), 8.35 (d, J=8.4 Hz, 1H), 7.90 (d, J=2.3 Hz, 1H), 7.83 (d, J=8.4 Hz, 1H), 7.40 (dd, J=12.6, 9.3 Hz, 1H), 7.31-7.26 (m, 1H), 7.25-7.21 (m, 1H), 7.18-7.14 (m, 1H), 7.19-7.06 (m, 1H), 7.04-6.98 (m, 1H), 5.62 (d, J=7.3 Hz, 2H), 5.12-5.02 (m, 1H), 2.31-2.22 (m, 1H), 2.01-1.94 (m, 2H), 1.50-1.35 (m, 4H), 1.38-1.25 (m, 1H), 0.95-0.87 (m, 2H), 0.71-0.63 (m, 2H).

Isomer 2 (I-590): ESI-MS [M+H]+: 461.2, ¹H NMR (400 MHz, DMSO) δ 8.56-8.52 (m, 2H), 8.05-7.94 (m, 2H), 7.59-7.51 (m, 1H), 7.37-7.06 (m, 5H), 5.79-5.75 (m, 2H), 5.11-5.04 (m, 1H), 2.31-2.24 (m, 1H), 2.04-1.91 (m, 2H), 1.44-1.31 (m, 4H), 1.26-1.20 (m, 1H), 1.02-0.95 (m, 2H), 0.76-0.68 (m, 2H).

### Example 591

### Synthesis of (1S,2S)-2-(3-chlorophenyl)-N-((1-((6-cyclopropyl-8-(4H-1,2,4-triazol-4-yl)imidazo[1,2-a]pyridin-2-yl)methyl)-1H-1,2,3-triazol-4-yl)methyl)cyclopropane-1-carboxamide (I-591)

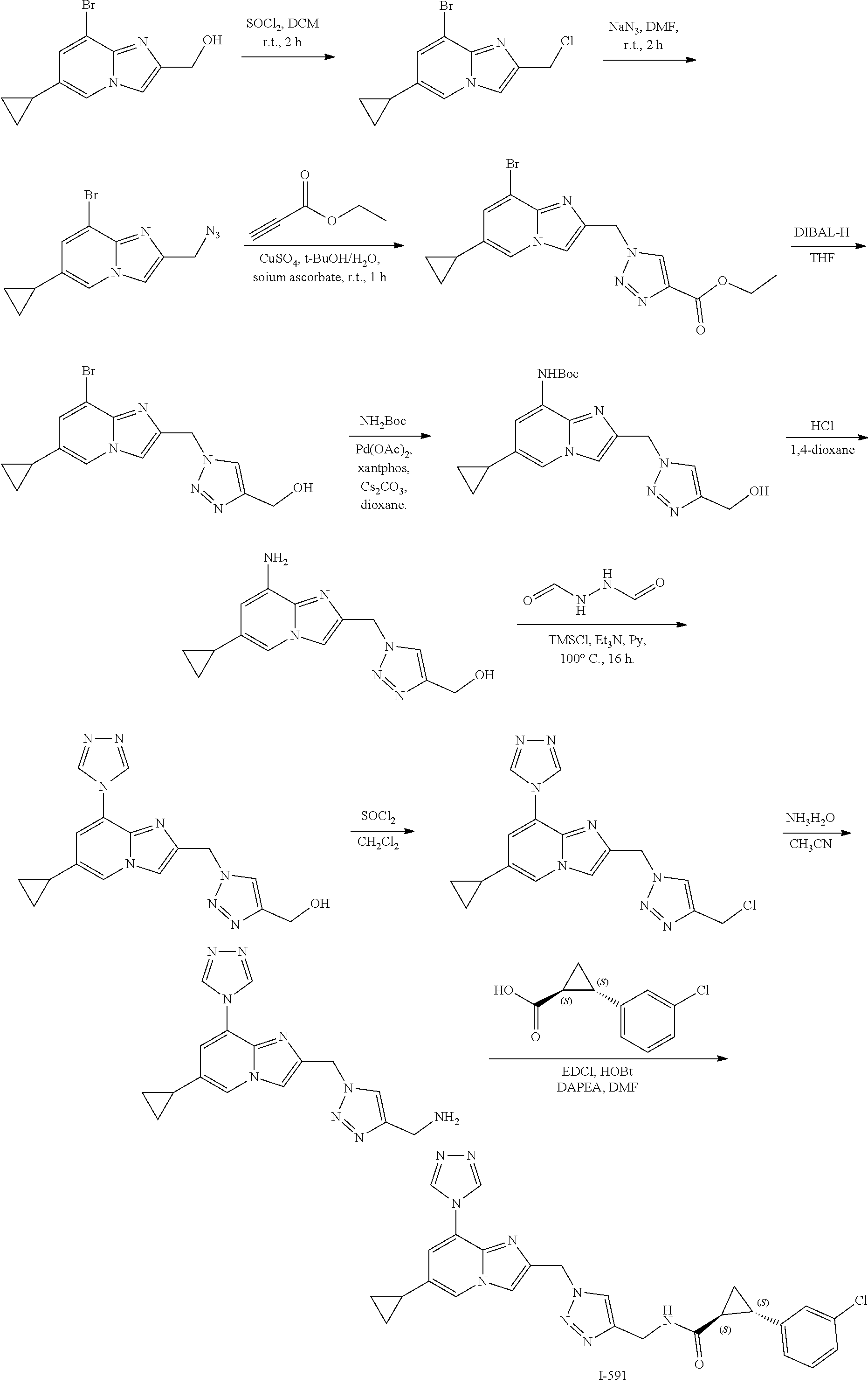

Synthesis of 8-bromo-2-(chloromethyl)-6-cyclopropylimidazo[1,2-a]pyridine. A solution of (8-bromo-6-cyclopropylimidazo[1,2-a]pyridin-2-yl)methanol (2 g, 7.5 mmol) and COCl₂ (2 mL) in DCM (25 mL) was stirred at room temperature for 2 h. The reaction was concentrated in vacuo to give 8-bromo-2-(chloromethyl)-6-cyclopropylimidazo[1,2-a]pyridine (2.2 g, crude) as yellow oil, which was used without further purification. ESI-MS [M+H]+: 285.2

Synthesis of 2-(azidomethyl)-8-bromo-6-cyclopropylimidazo[1,2-a]pyridine. To a solution of 8-bromo-2-(chloromethyl)-6-cyclopropylimidazo[1,2-a]pyridine (2.2 g, crude) in DMF (20 mL) was added NaN₃ (975 mg, 15 mmol). The resulting reaction mixture was stirred at room temperature for 2 h. The reaction was diluted with H₂O (200 mL) and extracted with EtOAc (100 mL×3). The combined organic layers were washed with brine (100 mL), dried over anhydrous Na₂SO₄ and concentrated in vacuo to give crude, which was purified by column chromatography (eluent: EtOAc/PE from 0 to 40%) to give 2-(azidomethyl)-8-bromo-6-cyclopropylimidazo[1,2-a]pyridine (1.8 g, 82% yield over 2 steps) ESI-MS [M+H]+: 292.2

Synthesis of ethyl ethyl 1-((8-bromo-6-cyclopropylimidazo[1,2-a]pyridin-2-yl)methyl)-1H-1,2,3-triazole-4-carboxylate. To a mixture of 2-(azidomethyl)-8-bromo-6-cyclopropylimidazo[1,2-a]pyridine (1.8 g, 6.2 mmol) in t-BuOH/H₂O (20 mL/20 mL) was added CuSO₄ (192 mg, 1.2 mmol) and sodium ascorbate (238 mg, 1.2 mmol). The resulting reaction was stirred at room temperature for 1 h. The reaction was diluted with H₂O (50 mL) and extracted with EtOAc (50 mL×3). The combined organic layers were washed with brine (50 mL), dried over anhydrous Na₂SO₄, and concentrated in vacuo to give the crude, which was purified by column chromatography (eluent: EtOAc/PE from 0 to 50%) to give ethyl 1-((8-bromo-6-cyclopropylimidazo[1,2-a]pyridin-2-yl)methyl)-1H-1,2,3-triazole-4-carboxylate (2.1 g, 88%) as a yellow solid. ESI-MS [M+H]+: 390.2

Synthesis of (1-((8-bromo-6-cyclopropylimidazo[1,2-a]pyridin-2-yl)methyl)-1H-1,2,3-triazol-4-yl)methanol. To a solution of ethyl 1-((8-bromo-6-cyclopropylimidazo[1,2-a]pyridin-2-yl)methyl)-1H-1,2,3-triazole-4-carboxylate (828 mg, 2.13 mmol) in dry THF (20 mL) at −60° C. was added DIBAL-H (6.39 mL, 6.39 mmol, 1M in THF) under N₂, The mixture was stirred at −60° C. for 1 h and then warmed to room temperature for 2 h. The mixture was quenched with NH₄Cl (sat·aq·, 50 mL), filtered through Celite®, and the filter cake was washed with EtOAc (50 mL×3). The filtrate was extracted with EtOAc (50 mL×3). The combined organic layers were washed with brine (50 mL), dried over Na₂SO₄ and concentrated in vacuo. The residue was purified by column chromatography (eluent: DCM/MeOH=0-5%) to give (1-((8-bromo-6-cyclopropylimidazo[1,2-a]pyridin-2-yl)methyl)-1H-1,2,3-triazol-4-yl)methanol as a white solid (600 mg, 81%). ESI-MS [M+H]+: 348.1.

Synthesis of tert-butyl tert-butyl tert-butyl (6-cyclopropyl-2-((4-(hydroxymethyl)-1H-1,2,3-triazol-1-yl)methyl)imidazo[1,2-a]pyridin-8-yl)carbamate. To a mixture of (1-((8-bromo-6-cyclopropylimidazo[1,2-a]pyridin-2-yl)methyl)-1H-1,2,3-triazol-4-yl)methanol (450 mg, 1.30 mmol) in dioxane (10 mL) was added tert-butyl carbamate (228 mg, 1.95 mmol), Pd(OAc)₂ (29 mg, 0.13 mmol), Xantphos (75 mg, 0.13 mmol), and Cs₂CO₃ (1.27 g, 3.9 mmol), and the mixture was stirred at 95° C. for 16 h under N₂. After cooling to room temperature, the reaction mixture was filtered through Celite® and the filter cake was washed with DCM/MeOH (10/1, 50 mL). The filtrate was concentrated in vacuo to give the crude, which was purified by column chromatography (eluent: DCM/MeOH=0-3%) to give (6-cyclopropyl-2-((4-(hydroxymethyl)-1H-1,2,3-triazol-1-yl)methyl)imidazo[1,2-a]pyridin-8-yl)carbamate as a white solid (90 mg, 18%). ESI-MS [M+H]+: 385.2.

Synthesis of (1-((8-amino-6-cyclopropylimidazo[1,2-a]pyridin-2-yl)methyl)-1H-1,2,3-triazol-4-yl)methanol. A solution of (6-cyclopropyl-2-((4-(hydroxymethyl)-1H-1,2,3-triazol-1-yl)methyl)imidazo[1,2-a]pyridin-8-yl)carbamate (90 mg, 0.23 mmol) in HCl (5.0 mL, 4M in dioxane) was stirred at room temperature for 1 h. The mixture was concentrated to give (1-((8-amino-6-cyclopropylimidazo[1,2-a]pyridin-2-yl)methyl)-1H-1,2,3-triazol-4-yl)methanol (66 mg, crude) as a yellow solid which was used for the next step directly without purification. ESI-MS [M+H]+: 285.1.

Synthesis of (1-((6-cyclopropyl-8-(4H-1,2,4-triazol-4-yl)imidazo[1,2-a]pyridin-2-yl)methyl)-1H-1,2,3-triazol-4-yl)methanol. To a mixture of N′-formylformohydrazide (280 mg, 3.18 mmol) and TEA (749 mg, 7.42 mmol) and (1-((8-amino-6-cyclopropylimidazo[1,2-a]pyridin-2-yl)methyl)-1H-1,2,3-triazol-4-yl)methanol (300 mg, 1.06 mmol) in pyridine (10 mL) was added TMSCl (1.72 g, 15.9 mmol) at 0° C. under N₂. The resulting mixture was stirred at 100° C. for 18 h. After cooling to room temperature, the reaction mixture was quenched with water (100 mL) and extracted with EtOAc (50 mL×3). The combined organic layers were washed with brine (50 mL), dried over anhydrous Na₂SO₄ and concentrated in vacuo to give the crude product, which was purified by column chromatography (eluent: DCM/MeOH=20/1˜10/1) to give (1-((6-cyclopropyl-8-(4H-1,2,4-triazol-4-yl)imidazo[1,2-a]pyridin-2-yl)methyl)-1H-1,2,3-triazol-4-yl)methanol (150 mg, 42%) as a white solid. ESI-MS [M+H]+: 337.1.

Synthesis of 2-((4-(chloromethyl)-1H-1,2,3-triazol-1-yl)methyl)-6-cyclopropyl-8-(4H-1,2,4-triazol-4-yl)imidazo[1,2-a]pyridine. A mixture of (1-((6-cyclopropyl-8-(4H-1,2,4-triazol-4-yl)imidazo[1,2-a]pyridin-2-yl)methyl)-1H-1,2,3-triazol-4-yl)methanol (33 mg, 0.10 mmol) in SOCl₂/CH₂Cl₂ (0.5 mL/2.5 mL) was stirred at 0° C. for 1 h, then at 35° C. for 1.5 h. The mixture was cooled then concentrated in vacuo to give (1-((6-cyclopropyl-8-(4H-1,2,4-triazol-4-yl)imidazo[1,2-a]pyridin-2-yl)methyl)-1H-1,2,3-triazol-4-yl)methanamine (30 mg, crude) as a yellow solid, which was used without purification. ESI-MS [M+H]+: 355.1.

Synthesis of (1-((6-cyclopropyl-8-(4H-1,2,4-triazol-4-yl)imidazo[1,2-a]pyridin-2-yl)methyl)-1H-1,2,3-triazol-4-yl)methanamine. A mixture of 2-((4-(chloromethyl)-1H-1,2,3-triazol-1-yl)methyl)-6-cyclopropyl-8-(4H-1,2,4-triazol-4-yl)imidazo[1,2-a]pyridine (36 mg, 0.10 mmol) in NH₄OH/CH₃CN (2.5 mL/0.5 mL) was stirred in a sealed in tube at room temperature for 16 h. Then, the mixture was concentrated in vacuo to give (1-((6-cyclopropyl-8-(4H-1,2,4-triazol-4-yl)imidazo[1,2-a]-pyridin-2-yl)methyl)-1H-1,2,3-triazol-4-yl)methanamine (30 mg, crude) as a yellow solid, which was used without purification. ESI-MS [M+H]+: 336.1.

Synthesis of (1S,2S)-2-(3-chlorophenyl)-N-((1-((6-cyclopropyl-8-(4H-1,2,4-triazol-4-yl)imidazo[1,2-a]pyridin-2-yl)methyl)-1H-1,2,3-triazol-4-yl)methyl)cyclopropane-1-carboxamide (I-591). To a solution of (1-((6-cyclopropyl-8-(4H-1,2,4-triazol-4-yl)imidazo[1,2-a]pyridin-2-yl)methyl)-1H-1,2,3-triazol-4-yl)methanamine (30 mg, 0.1 mmol) in DMF (2.0 mL) were added (1S,2S)-2-(3-chlorophenyl)cyclopropane-1-carboxylic acid (20 mg. 0.1 mmol), HOBt (20 mg, 0.15 mmol), and DIPEA (39 mg, 0.3 mmol) at room temperature. After stirring at room temperature for 16 h, the reaction was diluted with water (20 mL) and extracted with EtOAc (3×40 mL). The combined organic layers were washed with brine (30 mL), dried over Na₂SO₄ and concentrated in vacuo to give the crude, which was purified by preparative HPLC to give (1S,2S)-2-(3-chlorophenyl)-N-((1-((6-cyclopropyl-8-(4H-1,2,4-triazol-4-yl)imidazo[1,2-a]pyridin-2-yl)methyl)-1H-1,2,3-triazol-4-yl)methyl)cyclopropane-1-carboxamide (6 mg, 11%) as a white solid. ESI-MS [M+H]+: 514.2, ¹H NMR (400 MHz, DMSO) δ 9.40 (s, 2H), 8.58 (t, J=5.5 Hz, 1H), 8.48 (s, 1H), 8.01 (d, J=3.3 Hz, 2H), 7.45 (s, 1H), 7.31-7.20 (m, 2H), 7.17 (s, 1H), 7.08 (d, J=7.6 Hz, 1H), 5.71 (s, 2H), 4.36-4.28 (m, 2H), 2.33-2.24 (m, 1H), 2.02-1.96 (m, 1H), 1.95-1.86 (m, 1H), 1.38-1.33 (m, 1H), 1.26-1.20 (m, 1H), 1.05-0.93 (m, 2H), 0.89-0.76 (m, 2H).

### Example 592

### Synthesis of rac-(1S*, 2S*)-2-(3-chlorophenyl)-N-(2-((6-cyclopropylimidazo[1,2-a]pyridin-2-yl)methyl)-2H-pyrazolo[3,4-c]pyridin-7-yl)cyclopropane-1-carboxamide (I-592)

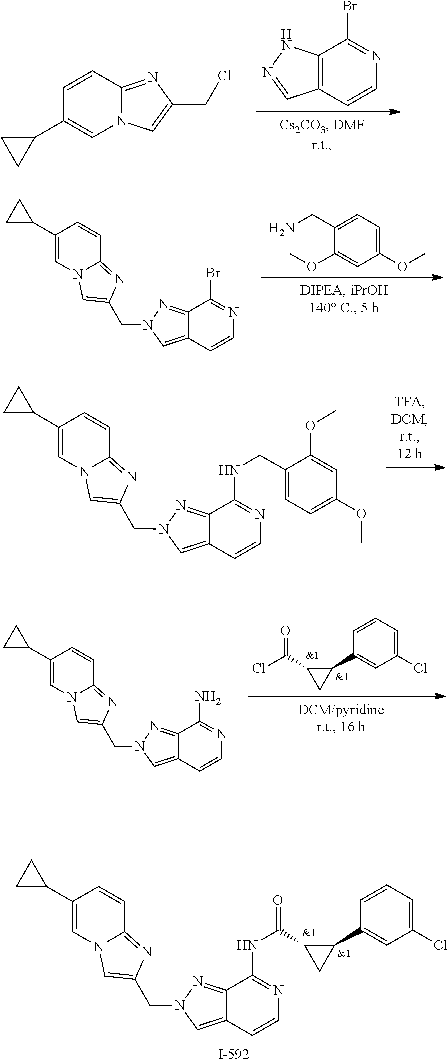

Synthesis of 7-bromo-2-((6-cyclopropylimidazo[1,2-a]pyridin-2-yl)methyl)-2H-pyrazolo[3,4-c]pyridine. To a solution of 2-(chloromethyl)-6-cyclopropylimidazo[1,2-a]pyridine (380 mg, 1.84 mmol) in DMF (15 mL) was added Cs₂CO₃ (1.79 g, 5.5 mmol) and 7-bromo-1H-pyrazolo[3,4-c]pyridine (302 mg, 1.52 mmol). The mixture was stirred at rt for 16 h. Water (30 mL) was added and extracted by EtOAc (30 mL×3). The combined organic layers were washed with brine, dried over Na₂SO₄, filtered, then concentrated in vacuo. The residue was purified by preparative TLC (EA:PE=1:1) to give the product 7-bromo-2-((6-cyclopropylimidazo[1,2-a]pyridin-2-yl)methyl)-2H-pyrazolo[3,4-c]pyridine (300 mg, 44%) as a white solid. ESI-MS [M+H]+: 369.1.

Synthesis of 2-((6-cyclopropylimidazo[1,2-a]pyridin-2-yl)methyl)-N-(2,4-dimethoxybenzyl)-2H-pyrazolo[3,4-c]pyridin-7-amine. A mixture of 7-bromo-2-((6-cyclopropylimidazo[1,2-a]pyridin-2-yl)methyl)-2H-pyrazolo[3,4-c]pyridine (100 mg, 0.27 mmol), (2,4-dimethoxyphenyl)methanamine (68 mg, 0.41 mmol), and DIPEA (104 mg, 0.81 mmol) in iPrOH (2 mL) was degassed for 1 min and stirred in a sealed tube. The reaction mixture was irradiated in microwave at 140° C. for 5 h and then concentrated in vacuo to give the crude product, which was purified by preparative TLC (eluent: DCM/MeOH=10/1) to give 2-((6-cyclopropylimidazo[1,2-a]pyridin-2-yl)methyl)-N-(2,4-dimethoxybenzyl)-2H-pyrazolo[3,4-c]pyridin-7-amine (100 mg, 81%) as a yellow oil. ESI-MS [M+H]⁺: 455.3.

Synthesis of 2-((6-cyclopropylimidazo[1,2-a]pyridin-2-yl)methyl)-2H-pyrazolo[3,4-c]pyridin-7-amine. To a mixture of 2-((6-cyclopropylimidazo[1,2-a]pyridin-2-yl)methyl)-N-(2,4-dimethoxybenzyl)-2H-pyrazolo[3,4-c]pyridin-7-amine (100 mg, 0.26 mmol) in DCM (2 mL) was added TFA (1 mL). After stirring at room temperature for 12 h, the reaction solution was diluted with DCM (20 mL) and washed with NaHCO₃ (10 mL). The organic layer was dried over anhydrous Na₂SO₄ then concentrated in vacuo to give the crude product, which was purified by preparative TLC (eluent: DCM/MeOH=10/1, 1% of NH₃ as the additive) to give 2-((6-cyclopropylimidazo[1,2-a]pyridin-2-yl)methyl)-2H-pyrazolo[3,4-c]pyridin-7-amine (70 mg, 88%) as a yellow oil ESI-MS [M+H]+: 305.2.

Synthesis of rac-(1S*,2S*)-2-(3-chlorophenyl)-N-(2-((6-cyclopropylimidazo[1,2-a]pyridin-2-yl)methyl)-2H-pyrazolo[3,4-c]pyridin-7-yl)cyclopropane-1-carboxamide (I-592). To a mixture of 2-((6-cyclopropylimidazo[1,2-a]pyridin-2-yl)methyl)-2H-pyrazolo[3,4-c]pyridin-7-amine (50 mg, 0.16 mmol) in DCM (2 mL) was added pyridine (1 mL) and DMAP (2 mg, 0.016 mmol). After stirring the mixture at room temperature for 20 min, a solution of rac-(1S*,2S*)-2-(3-chlorophenyl)cyclopropane-1-carbonyl chloride (39 mg, 0.18 mmol) in DCM (1 mL) was added dropwise and stirred at room temperature for 16 h. The reaction was diluted with DCM (20 mL) and washed sequentially with water (2×10 mL) and brine (10 mL). The organic extract was dried over anhydrous Na₂SO₄ and concentrated in vacuo to give the crude product, which was purified by preparative TLC (eluent: DCM/MeOH=10/1) to give rac-(1S*,2S*)-2-(3-chlorophenyl)-N-(2-((6-cyclopropylimidazo[1,2-a]pyridin-2-yl)methyl)-2H-pyrazolo[3,4-c]pyridin-7-yl)cyclopropane-1-carboxamide (11 mg, 14%) as a white solid. ESI-MS [M+H]+: 483.2, ¹H NMR (400 MHz, DMSO) δ 10.54 (s, 1H), 8.53 (s, 1H), 8.33 (s, 1H), 8.24 (s, 1H), 7.86-7.72 (m, 2H), 7.40-7.38 (m, 2H), 7.34-7.30 (m, 1H), 7.27-7.25 (m, 2H), 7.19-7.17 (m, 1H), 7.01-6.98 (m, 1H), 5.78 (s, 2H), 2.44-2.41 (m, 2H), 1.92-1.91 (m, 1H), 1.51-1.50 (m, 1H), 1.38-1.37 (m, 1H), 0.92-0.90 (m, 2H), 0.67-0.65 (m, 2H).

### Example 593

### Synthesis of rac-(1S*,2S*)-2-(3-chlorophenyl)-N-(1-((6-cyclopropylimidazo[1,2-a]pyridin-2-yl)methyl)-1H-1,2,3-triazol-4-yl)cyclopropane-1-carboxamide (I-593)

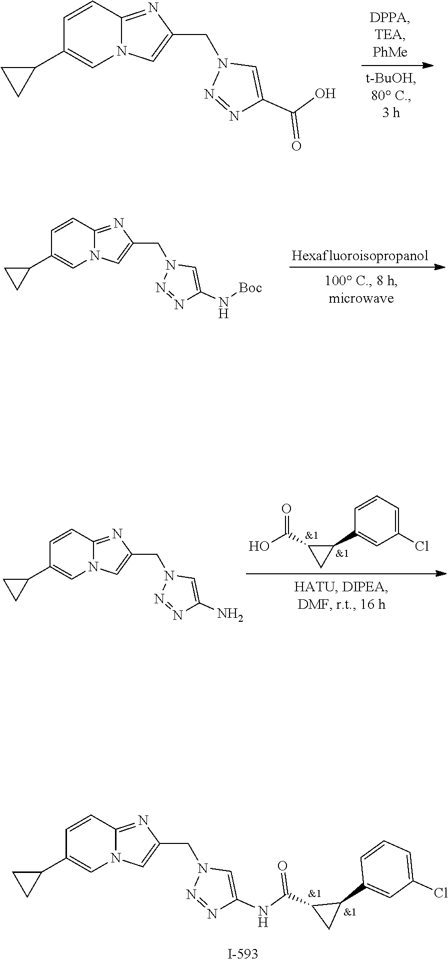 [SMILES: O=C(O)c1cn(Cc2cn3cc(C4CC4)ccc3n2)nn1 ; CC(C)(C)OC(=O)Nc1cn(Cc2cn3cc(C4CC4)ccc3n2)nn1 ; Nc1cn(Cc2cn3cc(C4CC4)ccc3n2)nn1 ; O=C(O)[C@H]1C[C@@H]1c1cccc(Cl)c1 ; O=C(Nc1cn(Cc2cn3cc(C4CC4)ccc3n2)nn1)[C@H]1C[C@@H]1c1cccc(Cl)c1]

Synthesis of tert-butyl (1-((6-cyclopropylimidazo[1,2-a]pyridin-2-yl)methyl)-1H-1,2,3-triazol-4-yl)carbamate. A mixture of 1-((6-cyclopropylimidazo[1,2-a]pyridin-2-yl)methyl)-1H-1,2,3-triazole-4-carboxylic acid (1 g, 3.53 mmol), TEA (400 mg, 3.88 mmol), and DPPA (1.04 g, 3.88 mmol) in toluene (10 mL) was stirred at 80° C. for 2 h. Then tBuOH (3 mL) was added and the resulting reaction was stirred at 80° C. for another 3 h. After cooling to room temperature, the mixture was washed with saturated aq·NaCl (25 mL) and extracted with EtOAc (3×30 mL). The combined organic layers were dried over Na₂SO₄, concentrated in vacuo, and the residue was purified by column chromatography (eluent: MeOH/DCM=1/50) to give tert-butyl(1-((6-cyclopropylimidazo[1,2-a]pyridin-2-yl)methyl)-1H-1,2,3-triazol-4-yl)carbamate (140 mg, 11%) as a white solid. ESI-MS [M+H]+: 355.2

Synthesis of 1-((6-cyclopropylimidazo[1,2-a]pyridin-2-yl)methyl)-JH-1,2,3-triazol-4-amine. A mixture of tert-butyl (1-((6-cyclopropylimidazo[1,2-a]pyridin-2-yl)methyl)-1H-1,2,3-triazol-4-yl)carbamate (140 mg, 0.395 mmol) in hexafluoroisopropanol (6 mL) was degassed with N₂ for 1 min, then stirred in a sealed tube. After heating in microwave at 100° C. for 8 h, the mixture was concentrated in vacuo to give the crude product, which was purified by column chromatography (eluent: MeOH/DCM=1/20) to give 1-((6-cyclopropylimidazo[1,2-a]pyridin-2-yl)methyl)-1H-1,2,3-triazol-4-amine (70 mg, 71%) as a yellow solid. ESI-MS [M+H]+: 255.1

Synthesis of rac-(1S*,2S*)-2-(3-chlorophenyl)-N-(1-((6-cyclopropylimidazo[1,2-a]pyridin-2-yl)methyl)-1H-1,2,3-triazol-4-yl)cyclopropane-1-carboxamide (I-593). A mixture of 1-((6-cyclopropylimidazo[1,2-a]pyridin-2-yl)methyl)-1H-1,2,3-triazol-4-amine (70 mg, 0.27 mmol), rac-(1S*,2S*)-2-(3-chlorophenyl)cyclopropane-1-carboxylic acid (63 mg, 0.22 mmol), HATU (132 mg, 0.33 mmol), and DIPEA (120 mg, 0.89 mmol)) in DMF (5 mL) was stirred at room temperature for 16 h. The reaction was diluted with water (20 mL) and extracted with EtOAc (3×30 mL). The combined organic layers were washed with brine (30 mL), dried over anhydrous Na₂SO₄, then concentrated in vacuo to give the crude, which was purified by column chromatography (eluent: MeOH/DCM=1/20) to give rac-(1S*,2S*)-2-(3-chlorophenyl)-N-(1-((6-cyclopropylimidazo[1,2-a]pyridin-2-yl)methyl)-1H-1,2,3-triazol-4-yl)cyclopropane-1-carboxamide (6.6 mg, 5%) as a white solid. ESI-MS [M+H]+: 433.1, ¹H NMR (400 MHz, DMSO) δ=11.15 (s, 1H), 8.35 (s, 1H), 8.11 (s, 1H), 7.83 (s, 1H), 7.42 (d, J=9.3, 1H), 7.34-7.28 (m, 1H), 7.28-7.21 (m, 2H), 7.14 (d, J=7.6, 1H), 7.02 (d, J=9.5, 1H), 5.63 (s, 2H), 2.42-2.32 (m, 1H), 2.23-2.18 (m, 1H), 1.96-1.88 (m, 1H), 1.49-1.37 (m, 2H), 0.97-0.88 (m, 2H), 0.72-0.64 (m, 2H).

### Example 594

### Synthesis of rac-(1S*,2S*)-2-(3-chlorophenyl)-N-(1-((6-cyclopropylimidazo[1,2-a]pyridin-2-yl)methyl)-1H-imidazo[4,5-c]pyridin-6-yl)cyclopropane-1-carboxamide (I-594)

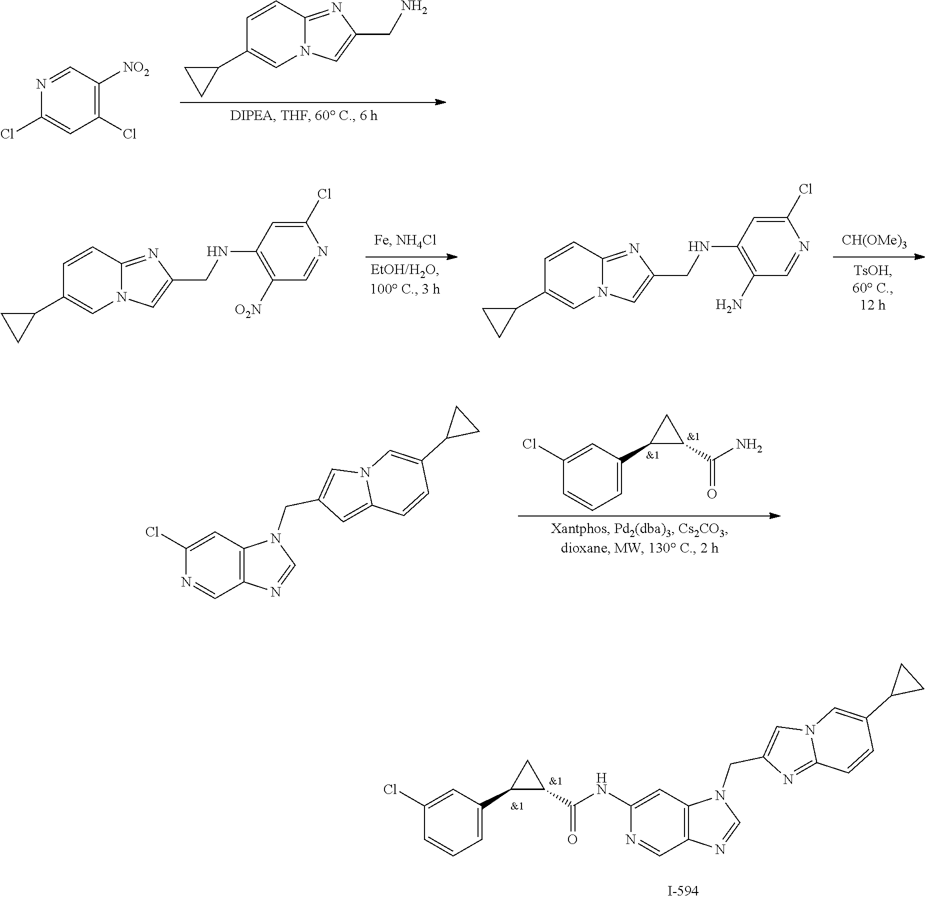

Synthesis of 2-chloro-N-((6-cyclopropylimidazo[1,2-a]pyridin-2-yl)methyl)-5-nitropyridin-4-amine. To a solution of 2,4-dichloro-5-nitropyridine (1.0 g, 5.2 mmol) in THF (30 mL) were added (6-cyclopropylimidazo[1,2-a]pyridin-2-yl)methanamine (2.0 g, 8.9 mmol) and DIPEA (2.7 g, 20.8 mmol). The resulting mixture was heated to 60° C. and stirred for 6 h. The reaction was quenched with water (100 ml) and extracted with EtOAc (3×100 mL). The combined organic layers were washed with brine, dried over Na₂SO₄ and concentrated in vacuo to give the crude product, which was purified by silica gel column chromatography (eluent: MeOH/DCM=1/20) to give the product (1.6 g, 90%) as a yellow solid. LC-MS: (M+H)⁺ 344.0.

Synthesis of 6-chloro-N4-((6-cyclopropylimidazo[1,2-a]pyridin-2-yl)methyl)pyridine-3,4-diamine. A solution of 2-chloro-N-((6-cyclopropylimidazo[1,2-a]pyridin-2-yl)methyl)-5-nitropyridin-4-amine (500 mg, 1.45 mmol) in ethanol (20 mL) was heated at 100° C., then sequentially were added iron (820 mg, 14.5 mmol) and a solution of NH₄Cl (780 mg, 14.5 mmol) in water (6 ml). The resulting suspension mixture was stirred at 100° C. for 3 h. The hot reaction mixture was filtered through a Celite® pad and the filtrate was concentrated in vacuo. The residue was dissolved in EtOAc (30 mL) and washed with water (30 mL). The aqueous phase was further extracted with i-PrOH:CHCl3 (1:3, 3×50 mL). The combined organic layers were washed with brine, dried over Na₂SO₄ and concentrated in vacuo to give the crude product (450 mg, 99%) as a yellow solid. LC-MS: (M+H)⁺ 314.0

Synthesis of 6-chloro-1-((6-cyclopropylimidazo[1,2-a]pyridin-2-yl)methyl)-1H-imidazo[4,5-c]pyridine. To a solution of 6-chloro-N4-((6-cyclopropylimidazo[1,2-a]pyridin-2-yl)methyl)pyridine-3,4-diamine (450 mg, 1.44 mmol) in CH(OMe)₃ (10 mL) was added p-MeC₆H₄SO₃H (49.5 mg, 1.45 mmol). After stirring for 2 h at 60° C., the reaction was quenched with saturated aq·NaHCO₃ (50 mL) and extracted with EtOAc (3×50 mL). The combined organic layers were washed with brine, dried over Na₂SO₄ and concentrated in vacuo to give the crude product, which was purified by silica gel column chromatography (eluent: MeOH/DCM=1/10) to give the product (320 mg, 34%) as a yellow solid. LC-MS: (M+H)⁺ 324.0

Synthesis of rac-(1S*,2S*)-2-(3-chlorophenyl)-N-(1-((6-cyclopropylimidazo[1,2-a]pyridin-2-yl)methyl)-1H-imidazo[4,5-c]pyridin-6-yl)cyclopropane-1-carboxamide (I-594). To a solution of 6-chloro-1-((6-cyclopropylimidazo[1,2-a]pyridin-2-yl)methyl)-1H-imidazo-[4,5-c]pyridine (50 mg, 0.155 mmol) in 1,4-dioxane (2 mL) was added rac-(1S*,2S*)-2-(3-chlorophenyl)cyclopropane-1-carboxamide (30.2 mg, 0.155 mmol), Cs₂CO₃ (150 mg, 0.465 mmol), xantphos (9 mg, 0.0155 mmol), and Pd₂(dba)₃ (25.2 mg, 0.0155 mmol). The reaction was degassed with N₂ for 3 min, then irradiated in a microwave reactor at 130° C. for 2 h. The reaction was concentrated in vacuo to give the crude product, which was purified with preparative HPLC to give the product as a white solid (30 mg, 40%). ESI-MS [M+H]+: 483.2. ¹H NMR (400 MHz, DMSO) δ 10.77 (s, 1H), 8.68 (s, 1H), 8.41 (s, 1H), 8.33 (s, 1H), 8.29 (s, 1H), 7.71 (s, 1H), 7.38 (d, J=9.3 Hz, 1H), 7.34-7.30 (m, 1H), 7.26-7.25 (m, 2H), 7.15 (d, J=7.6 Hz, 1H), 7.00-6.98 (m, 1H), 5.54 (s, 2H), 2.41-2.38 (m, 2H), 1.93-1.88 (m, 1H), 1.50-1.37 (m, 2H), 0.93-0.88 (m, 2H), 0.68-0.64 (m, 2H)

### Example 595

### Synthesis of (1S,2S)-2-(3-chlorophenyl)-N-(4-(((6-cyclopropyl-8-((methylamino)methyl)imidazo[1,2-a]pyridin-2-yl)methyl)amino)pyridin-2-yl)cyclopropane-1-carboxamide (I-595)

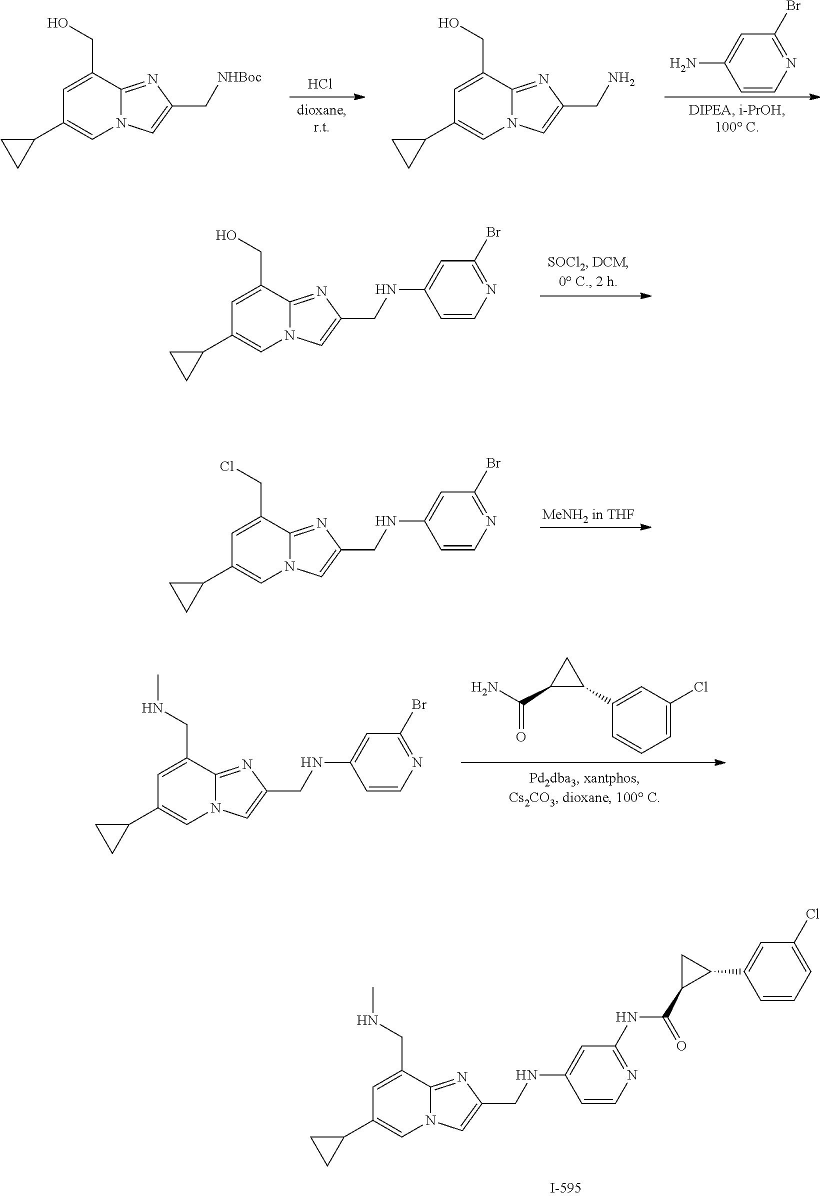

Synthesis of (2-(aminomethyl)-6-cyclopropylimidazo[1,2-a]pyridin-8-yl)methanol. A mixture of tert-butyl ((6-cyclopropyl-8-(hydroxymethyl)imidazo[1,2-a]pyridin-2-yl)methyl)-carbamate (317 mg, 1.0 mmol) in HCl (4M in dioxane, 5.0 mL) was stirred at room temperature for 2 h. The mixture was concentrated in vacuo to give (2-(aminomethyl)-6-cyclopropylimidazo-[1,2-a]pyridin-8-yl)methanol (220 mg, crude) as a yellow solid, which was used into the next step directly. ESI-MS [M+H]+: 218.2.

Synthesis of (2-(((2-bromopyridin-4-yl)amino)methyl)-6-cyclopropylimidazo[1,2-a]pyridin-8-yl)methanol. To a mixture of (2-(aminomethyl)-6-cyclopropylimidazo[1,2-a]pyridin-8-yl)methanol (217 mg, 1.0 mmol), 2-bromopyridin-4-amine (262.5 mg, 1.5 mmol), and DIPEA (387 mg, 3.0 mml) in i-PrOH (8 mL) was stirred at 100° C. for 16 h. The mixture was concentrated in vacuo and purified by preparative TLC (DCM/MeOH=15/1) to give the product (2-(((2-bromopyridin-4-yl)amino)methyl)-6-cyclopropylimidazo[1,2-a]pyridin-8-yl)methanol (400 mg, 82%) as yellow oil. ESI-MS [M+H]⁺: 373.2.

Synthesis of 2-bromo-N-((8-(chloromethyl)-6-cyclopropylimidazo[1,2-a]pyridin-2-yl)methyl)pyridin-4-amine. To a mixture of (2-(((2-bromopyridin-4-yl)amino)methyl)-6-cyclopropylimidazo[1,2-a]pyridin-8-yl)methanol (300 mg, 0.81 mmol) in DCM (5.0 mL) was added SOCl₂ (1.0 mL) at 0° C. The reaction mixture was stirred at 0° C. for 2 h. The mixture was concentrated in vacuo to give the product 2-bromo-N-((8-(chloromethyl)-6-cyclopropylimidazo[1,2-a]pyridin-2-yl)methyl)pyridin-4-amine (300 mg, crude) as a yellow solid, which was used without further purification. ESI-MS [M+H]⁺: 391.2.

Synthesis of 2-bromo-N-((6-cyclopropyl-8-((methylamino)methyl)imidazo[1,2-a]pyridin-2-yl)methyl)pyridin-4-amine. A mixture of 2-bromo-N-((8-(chloromethyl)-6-cyclopropylimidazo[1,2-a]pyridin-2-yl)methyl)pyridin-4-amine (200 mg, 0.51 mmol) in MeNH₂ (2.0 M in THF, 10 mL) was stirred at 70° C. in a sealed tube for 48 h. The mixture was cooled to room temperature, then concentrated in vacuo and purified by preparative TLC (DCM/MeOH=10/1) to give the 2-bromo-N-((6-cyclopropyl-8-((methylamino)methyl)imidazo-[1,2-a]pyridin-2-yl)methyl)pyridin-4-amine (30 mg, 15%) as a yellow oil. ESI-MS [M+H]⁺: 387.2.

Synthesis of (1S,2S)-2-(3-chlorophenyl)-N-(4-(((6-cyclopropyl-8-((methylamino)methyl)imidazo[1,2-a]pyridin-2-yl)methyl)amino)pyridin-2-yl)cyclopropane-1-carboxamide (I-595). A mixture of 2-bromo-N-((6-cyclopropyl-8-((methylamino)methyl)imidazo[1,2-a]pyridin-2-yl)-methyl)pyridin-4-amine (19.3 mg, 0.05 mmol), (1S,2S)-2-(3-chlorophenyl)cyclopropane-1-carboxamide (9.8 mg, 0.05 mmol), Pd₂(dba)₃ (4.6 mg, 0.005 mmol), xantphos (2.9 mg, 0.005 mmol), and Cs₂CO₃ (48.9 mg, 0.15 mmol) in 1,4-dioxane (2.5 mL) was stirred at 100° C. under N₂ for 16 h. The mixture was cooled to room temperature, concentrated in vacuo and purified by preparative TLC (DCM/MeOH=7/1) to give (1S,2S)-2-(3-chlorophenyl)-N-(4-(((6-cyclopropyl-8-((methylamino)methyl)imidazo[1,2-a]pyridin-2-yl)methyl)amino)pyridin-2-yl)cyclopropane-1-carboxamide (5.2 mg, 21%) as a light yellow solid. ESI-MS [M+H]+: 501.2, ¹H NMR (400 MHz, DMSO) δ 10.32 (s, 1H), 8.26 (s, 1H), 7.76 (d, J=5.7 Hz, 1H), 7.65 (s, 1H), 7.44 (s, 1H), 7.30 (d, J=7.6 Hz, 1H), 7.26-7.24 (m, 2H), 7.13 (d, J=6.9 Hz, 2H), 6.98 (s, 1H), 6.32 (d, J=5.7 Hz, 1H), 4.37 (d, J=5.3 Hz, 2H), 4.04 (s, 2H), 2.41 (s, 3H), 2.39-2.23 (m, 2H), 1.97-1.81 (m, 1H), 1.51-1.40 (m, 1H), 1.39-1.29 (m, 1H), 0.97-0.87 (m, 2H), 0.68-0.65 (m, 2H).

### Example 596

### Synthesis of (1S,2S)-2-(3-chlorophenyl)-N-(5-((6-cyclopropylimidazo[1,2-a]pyridin-2-yl)methoxy)pyrimidin-4-yl)cyclopropane-1-carboxamide (I-596)

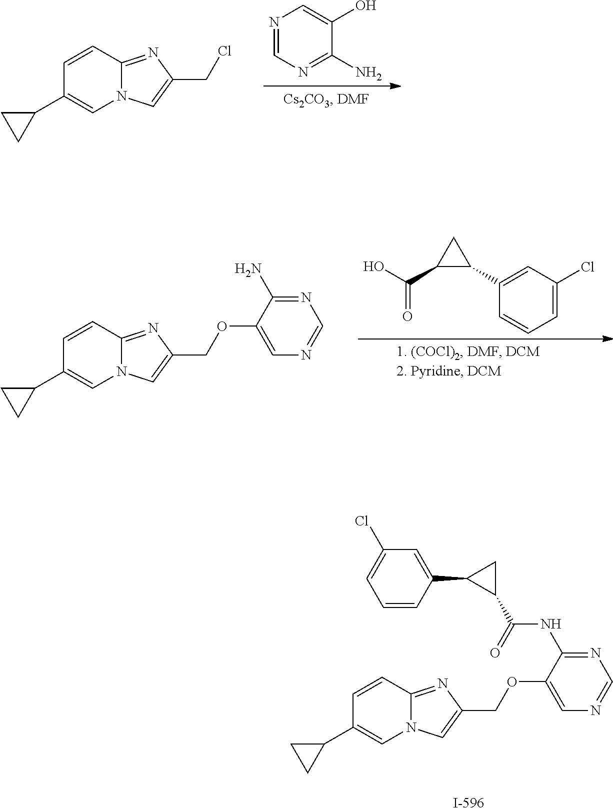 [SMILES: ClCc1cn2cc(C3CC3)ccc2n1 ; Nc1ncncc1O ; Nc1ncncc1OCc1cn2cc(C3CC3)ccc2n1 ; O=C(O)[C@H]1C[C@@H]1c1cccc(Cl)c1 ; O=C(Nc1ncncc1OCc1cn2cc(C3CC3)ccc2n1)[C@H]1C[C@@H]1c1cccc(Cl)c1]

Synthesis of 5-((6-cyclopropylimidazo[1,2-a]pyridin-2-yl)methoxy)pyrimidin-4-amine. A solution of 2-(chloromethyl)-6-cyclopropylimidazo[1,2-a]pyridine (50 mg, 0.45 mmol), 4-aminopyrimidin-5-ol (102 mg, 0.50 mmol), and Cs₂CO₃ (293 mg, 0.9 mmol) in DMF (10 mL) was stirred at 60° C. for 16 h. The reaction was quenched with water (30 mL) and extracted with EtOAc (3×30 mL). The combined organic layers were washed with brine, dried over Na₂SO₄, and concentrated in vacuo to give the crude, which was purified by silica gel chromatography (eluent: DCM/MeOH=10/1) to give the 5-((6-cyclopropylimidazo[1,2-a]pyridin-2-yl)methoxy)pyrimidin-4-amine as a yellow solid. (50 mg, 40%), ESI-MS [M+H]+: 282.2

Synthesis of (1S,2S)-2-(3-chlorophenyl)-N-(5-((6-cyclopropylimidazo[1,2-a]pyridin-2-yl)methoxy)pyrimidin-4-yl)cyclopropane-1-carboxamide (I-596). To a solution of (1S,2S)-2-(3-chlorophenyl)cyclopropane-1-carboxylic acid (50 mg, 0.6 mmol) in DCM (5 mL) was added oxalyl dichloride (65 mg, 0.52 mmol) and DMF (3 drops) at 0° C. under nitrogen. After stirring at 0° C. for 3 h, the mixture was evaporated to give the product as light-yellow oil (46 mg, crude), which was used without further purification.

To a solution of 5-((6-cyclopropylimidazo[1,2-a]pyridin-2-yl)methoxy)pyrimidin-4-amine (30 mg, 0.11 mmol) in pyridine (1 mL) in DCM (3 mL) was added a solution of (1S,2S)-2-(3-chlorophenyl)cyclopropane-1-carbonyl chloride (46 mg, 0.22 mmol) in DCM (2 mL). After stirring at room temperature for 12 h, the reaction was quenched with water (10 mL) and extracted with EtOAc (3×10 mL). The combined organic layers were washed with brine, dried over Na₂SO₄, concentrated in vacuo to give the crude, which was purified with preparative TLC (eluent: DCM/MeOH=10/1) to give (1S,2S)-2-(3-chlorophenyl)-N-(5-((6-cyclopropylimidazo[1,2-a]pyridin-2-yl)methoxy)pyrimidin-4-yl)cyclopropane-1-carboxamide as a white solid. (15 mg, 29%). ESI-MS [M+H]+: 460.1, ¹H NMR (400 MHz, DMSO) δ 10.32 (s, 1H), 8.66 (s, 1H), 8.51 (s, 1H), 8.36 (s, 1H), 7.92 (s, 1H), 7.45-7.37 (m, 1H), 7.36-7.20 (m, 3H), 7.14 (d, J=7.5 Hz, 1H), 7.06-6.95 (m, 1H), 5.38 (s, 2H), 2.58-2.56 (m, 1H), 2.45-2.38 (m, 1H), 1.97-1.91 (m, 1H), 1.54-1.47 (m, 1H), 1.45-1.38 (m, 1H), 0.96-0.90 (m, 2H), 0.72-0.66 (m, 2H).

### Example 597

### Synthesis of (1S,2S)-2-(3-chlorophenyl)-N-(1-((6-cyclopropylimidazo[1,2-a]pyridin-2-yl)methyl)-2,3-dihydro-1H-pyrido[3,4-b][1,4]oxazin-7-yl)cyclopropane-1-carboxamide (I-597)

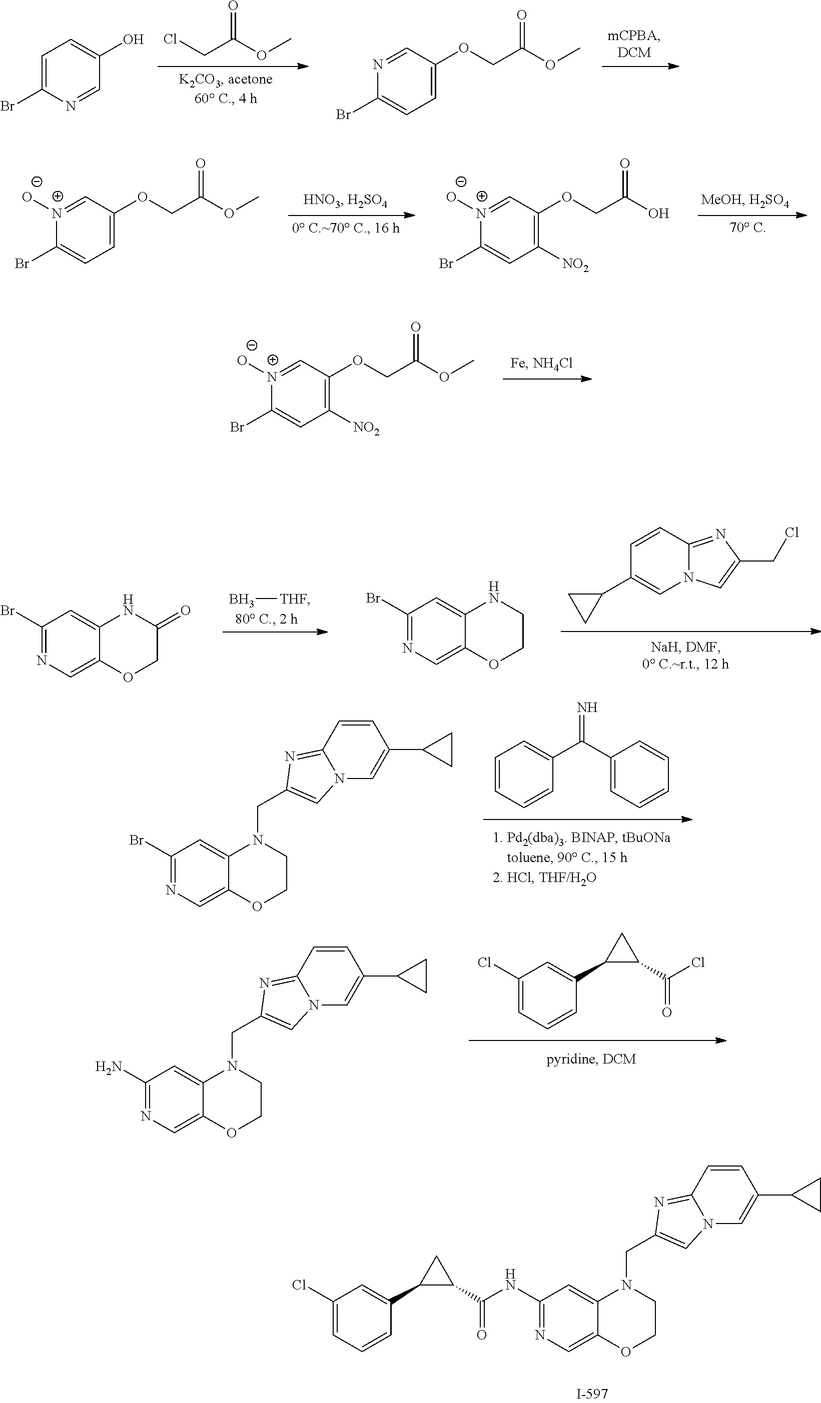

Synthesis of methyl 2-((6-bromopyridin-3-yl)oxy)acetate. To a mixture of 6-bromopyridin-3-ol (1.74 g, 10 mmol) and potassium carbonate (2.76 g, 20 mmol) in acetone (30 mL) was added dropwise chloroacetic methyl ester (1.08 g, 10 mmol). After stirring at 60° C. for 15 hours, the mixture was filtered, and the filtrate was concentrated in vacuo to afford the crude product, which was purified by flash silica gel chromatography (PE/EtOAc=1/1) to give methyl 2-((6-bromopyridin-3-yl)oxy)acetate (1.23 g, 50%) as a yellow solid. ESI-MS [M+H]+: 246.1.

Synthesis of 2-bromo-5-(2-methoxy-2-oxoethoxy)pyridine 1-oxide. To a mixture of methyl 2-(6-bromopyridin-3-yloxy)acetate (1.2 g, 5 mmol) in dichloromethane (20 mL) was added m-chloroperbenzoic acid (1.72 g, 10 mmol). After stirring at room temperature for 18 hours, reaction was quenched with saturated aq·sodium sulfite solution (50 mL) and extracted by DCM (3×40 mL). The combined organic layers were washed with brine, dried over Na₂SO₄ then concentrated in vacuo to give the crude product (0.9 g, crude) as a yellow solid, which was used without further purification. ESI-MS [M+H]+: 262.1.

Synthesis of 2-bromo-5-(carboxymethoxy)-4-nitropyridine 1-oxide. 2-bromo-5-(2-methoxy-2-oxoethoxy)pyridine 1-oxide (1 g, 3.8 mmol) was dissolved in sulfuric acid (4 mL) at 0° C., and then nitric acid (2 mL) was added slowly over several minutes. The reaction mixture was then placed in an oil bath heated to 40° C., then raised to 75° C. over 1 hour. After stirring at 75° C. for another 2 hours, the reaction mixture was slowly poured into the ice water and basified (pH=9) with saturated aq·NaHCO₃. The mixture was concentrated in vacuo to give the crude product (1.5 g, crude) as a white solid, which was used without further purification. ESI-MS [M+H]+: 294.1.

Synthesis of 2-bromo-5-(2-methoxy-2-oxoethoxy)-4-nitropyridine 1-oxide. To a mixture of 2-bromo-5-(carboxymethoxy)-4-nitropyridine 1-oxide (1.5 g, crude from previous step) in MeOH (100 mL) was added H₂SO₄ (3 mL). After stirring at 70° C. for 16 h, then mixture was filtered and washed with MeOH (100 mL). The filtrate was concentrated in vacuo to give the residue, which was re-dissolved in EtOAc (100 mL) and washed with saturated aq·NaHCO₃ (50 mL). The organic layer was washed with brine, dried over Na₂SO₄, and concentrated in vacuo to give crude, which was purified with silica gel chromatography (eluent: PE/EtOAc=1/1) to give 2-bromo-5-(2-methoxy-2-oxoethoxy)-4-nitropyridine 1-oxide (600 mg, 51% over two steps) as a yellow solid. ESI-MS [M+H]+: 307.1.

Synthesis of 7-bromo-1H-pyrido[3,4-b][1,4]oxazin-2(3H)-one. To a solution of 2-bromo-5-(2-methoxy-2-oxoethoxy)-4-nitropyridine 1-oxide (307 mg, 1 mmol) in MeOH (15 mL) was added Fe power (560 mg, 10 mmol) and NH₄Cl (540 mg, 10 mmol) in water (3 mL). After stirring at 70° C. for 2 h, the hot reaction mixture was filtered and washed with MeOH (50 mL). The filtrate was concentrated to give the residue, which was washed with saturated aq·NaHCO₃ (30 mL) and extracted by EtOAc (3×40 mL). The combined organic layers were washed with brine, dried over Na₂SO₄, and concentrated in vacuo to give the crude, which was purified with silica gel chromatography (eluent: PE/EtOAc=1/1) to give 7-bromo-1H-pyrido[3,4-b][1,4]oxazin-2(3H)-one (140 mg, 61%) as a white solid. ESI-MS [M+H]+: 229.1.

Synthesis of 7-bromo-2,3-dihydro-1H-pyrido[3,4-b][1,4]oxazine. To a solution of 7-bromo-1H-pyrido[3,4-b][1,4]oxazin-2(3H)-one (160 mg, 0.7 mmol) in THF (10 mL) was added BH₃-THF (1.68 mL, 1.68 mmol) at 0° C. The resulting mixture was stirred at 80° C. for 2 h and cooled to room temperature. The reaction was quenched with methanol (10 mL), washed with saturated aq·NaHCO₃ (20 mL), and extracted by EtOAc (3×30 mL). The combined organic layers was washed with brine, dried over Na₂SO₄, concentrated in vacuo, and purified by flash silica gel chromatography (eluent: PE/EtOAc=2/1) to give 7-bromo-2,3-dihydro-1H-pyrido[3,4-b][1,4]oxazine (80 mg, 50%) as a yellow solid. ESI-MS [M+H]+: 215.1.

Synthesis of 7-bromo-1-((6-cyclopropylimidazo[1,2-a]pyridin-2-yl)methyl)-2,3-dihydro-1H-pyrido[3,4-b][1,4]oxazine. To a solution of 7-bromo-2,3-dihydro-1H-pyrido[3,4-b][1,4]oxazine (65 mg, 0.30 mmol) in DMF (5 mL) was added NaH (60 mg, 1.51 mmol, 60% dispersion in mineral oil) at 0° C. and stirred at room temperature for 1 h. Then 2-(chloromethyl)-6-cyclopropylimidazo[1,2-a]pyridine (74 mg, 0.36 mmol) was added and the resulting mixture was stirred at room temperature for another 16 h. The reaction was quenched with saturated aq·NH₄Cl (20 mL) and extracted by EtOAc (3×20 mL). The combined organic layers were washed with brine, dried over Na₂SO₄, and concentrated in vacuo to give the crude, which was purified by silica gel chromatography (eluent: 100% of EtOAc) to give the product (90 mg, 77%) as a white solid. ESI-MS [M+H]+: 385.1.

Synthesis of 1-((6-cyclopropylimidazo[1,2-a]pyridin-2-yl)methyl)-2,3-dihydro-1H-pyrido[3,4-b][1,4]oxazin-7-amine. Step 1: A mixture of 7-bromo-1-((6-cyclopropylimidazo[1,2-a]pyridin-2-yl)methyl)-2,3-dihydro-1H-pyrido[3,4-b][1,4]oxazine (115 mg, 0.3 mmol), diphenylmethanimine (66 mg, 0.36 mmol), Pd₂(dba)₃ (57 mg, 0.06 mmol), BINAP (75 mg, 0.12 mmol) and t-BuOK (45 mg, 0.45 mmol) in toluene (4 mL) was stirred at 80° C. for 16 h. The reaction was washed with water (10 mL) and extracted by EtOAc (3×20 mL). The combined organic layers were washed with brine, dried over Na₂SO₄, and concentrated in vacuo to give the crude, which was purified by preparative TLC (DCM/MeOH=10/1) to give product (120 mg, 82%). ESI-MS [M+H]+: 486.1.

Step 2: To a solution of 7 N-(1-((6-cyclopropylimidazo[1,2-a]pyridin-2-yl)methyl)-2,3-dihydro-1H-pyrido[3,4-b][1,4]oxazin-7-yl)-1,1-diphenylmethanimine (100 mg, 0.206 mmol) in THF (6 mL) was added 1 N HCl (3 mL). After stirring at room temperature for 3 h, the pH of the reaction was adjusted to 8 by saturated aq·NaHCO₃ and extracted by EtOAc (3×30 mL). The combined organic layers were washed with brine, dried over Na₂SO₄, and concentrated in vacuo to give the crude, which was purified by preparative TLC (DCM/MeOH=10/1) to give product (50 mg, 75.7%) as a brown solid. ESI-MS [M+H]+: 322.1.

Synthesis of (1S,2S)-2-(3-chlorophenyl)-N-(1-((6-cyclopropylimidazo[1,2-a]pyridin-2-yl)methyl)-2,3-dihydro-1H-pyrido[3,4-b][1,4]oxazin-7-yl)cyclopropane-1-carboxamide (I-597). To a solution of 7-bromo-1-((6-cyclopropylimidazo[1,2-a]pyridin-2-yl)methyl)-2,3-dihydro-1H-pyrido[3,4-b][1,4]oxazine (50 mg, 0.16 mmol) in pyridine/DCM (1 mL/3 mL) was added a solution of (1S,2S)-2-(3-chlorophenyl)cyclopropane-1-carbonyl chloride (49 mg, 0.23 mmol) in DCM (1 mL) at 0° C. After stirring at room temperature for 5 h, the reaction was washed with water (10 mL) and extracted by DCM (3×20 mL). The combined organic layers were washed with brine, dried over Na₂SO₄, then concentrated in vacuo to give the crude, which was purified by preparative TLC (DCM/MeOH=10/1) to give (1S,2S)-2-(3-chlorophenyl)-N-(1-((6-cyclopropylimidazo[1,2-a]pyridin-2-yl)methyl)-2,3-dihydro-1H-pyrido[3,4-b][1,4]oxazin-7-yl)cyclopropane-1-carboxamide (10 mg, 13%) as a white solid. ESI-MS [M+H]+: 500.2, ¹H NMR (400 MHz, DMSO) δ 10.28 (s, 1H), 8.29 (s, 1H), 7.66 (s, 1H), 7.61 (s, 1H), 7.54 (s, 1H), 7.40 (d, J=9.3 Hz, 1H), 7.30 (t, J=7.7 Hz, 1H), 7.23 (dd, J=9.1, 1.2 Hz, 2H), 7.12 (d, J=7.6 Hz, 1H), 6.98 (dd, J=9.3, 1.7 Hz, 1H), 4.57 (s, 2H), 4.27-4.10 (m, 2H), 3.62-3.50 (m, 2H), 2.40-2.22 (m, 2H), 1.95-1.88 (m, 1H), 1.48-1.39 (m, 1H), 1.35-1.30 (m, 1H), 0.95-0.84 (m, 2H), 0.71-0.62 (m, 2H).

### Example 598

### Synthesis of (1S,2S)-2-(3-chlorophenyl)-N-(4-(((6-cyclopropyl-8-(2-(dimethylamino)ethyl)imidazo[1,2-a]pyridin-2-yl)methyl)amino)pyridin-2-yl)cyclopropane-1-carboxamide (I-598)

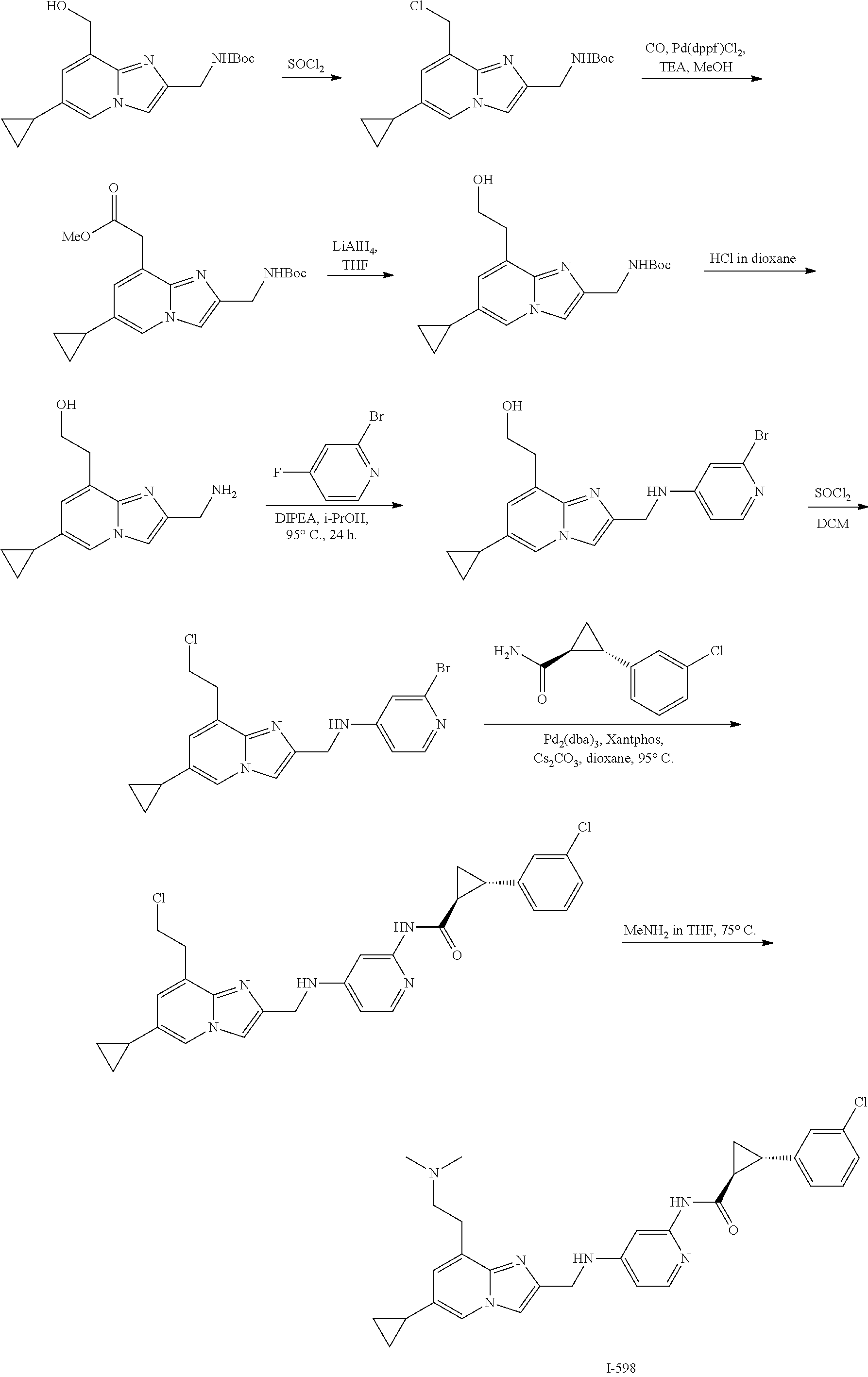

Synthesis of tert-butyl ((8-(chloromethyl)-6-cyclopropylimidazo[1,2-a]pyridin-2-yl)methyl)carbamate. To a mixture of tert-butyl ((6-cyclopropyl-8-(hydroxymethyl)imidazo[1,2-a]pyridin-2-yl)methyl)-carbamate (317 mg, 1.0 mmol) in DCM (5.0 mL) was added SOCl₂ (178.5 mg, 1.5 mmol) at 0° C. After stirring at 0° C. for 1 h, the reaction mixture was concentrated in vacuo to give tert-butyl ((8-(chloromethyl)-6-cyclopropylimidazo[1,2-a]pyridin-2-yl)methyl)carbamate (335 mg, crude) as a yellow solid, which was used without further purification. ESI-MS [M+H]+: 336.2.

Synthesis of methyl 2-(2-(((tert-butoxycarbonyl)amino)methyl)-6-cyclopropylimidazo[1,2-a-]pyridin-8-yl)acetate. A mixture of tert-butyl ((8-(chloromethyl)-6-cyclopropylimidazo[1,2-a]pyridin-2-yl)methyl)-carbamate (335 mg, 1.0 mmol) and Pd(dppf)Cl₂ (41 mg, 0.05 mmol) in TEA/MeOH (2.0 mL/10.0 mL) was stirred at 70° C. under CO atmosphere for 16 h. The mixture was concentrated in vacuo and purified by preparative TLC (DCM/MeOH=15/1) to give methyl 2-(2-(((tert-butoxycarbonyl)amino)-methyl)-6-cyclopropylimidazo[1,2-a]pyridin-8-yl)acetate (160 mg, 45%) as a yellow solid. ESI-MS [M+H]⁺: 360.2.

Synthesis of tert-butyl ((6-cyclopropyl-8-(2-hydroxyethyl)imidazo[1,2-a]pyridin-2-yl)methyl)carbamate. To a mixture of methyl 2-(2-(((tert-butoxycarbonyl)amino)methyl)-6-cyclopropylimidazo-[1,2-a]pyridin-8-yl)acetate (160 mg, 0.45 mmol) in THF (10.0 mL) was added a solution of LiAlH₄ (1.34 mL, 1.34 mmol, 1M solution in THF) at 0° C. under N₂. After stirring at 0° C. for 1 h, the mixture was quenched with water (0.2 mL), 15% NaOH solution (0.2 mL), and water (0.6 mL). The mixture was filtered and the filtrate concentrated in vacuo to give tert-butyl ((6-cyclopropyl-8-(2-hydroxyethyl)imidazo[1,2-a]pyridin-2-yl)methyl)carbamate (130 mg, crude) as a yellow solid, which was used without further purification. ESI-MS [M+H]⁺: 332.2.

Synthesis of 2-(2-(aminomethyl)-6-cyclopropylimidazo[1,2-a]pyridin-8-yl)ethan-1-ol. A mixture of tert-butyl ((6-cyclopropyl-8-(2-hydroxyethyl)imidazo[1,2-a]pyridin-2-yl)methyl)-carbamate (130 mg, 0.39 mmol) in HCl (4M in 1,4-dioxane, 2.0 mL) was stirred at room temperature for 1 h. The mixture was concentrated in vacuo to give 2-(2-(aminomethyl)-6-cyclopropylimidazo[1,2-a]pyridin-8-yl)ethan-1-ol (100 mg, crude) as a yellow solid, which was used without further purification. ESI-MS [M+H]⁺: 232.2.

Synthesis of 2-(2-(((2-bromopyridin-4-yl)amino)methyl)-6-cyclopropylimidazo[1,2-a]-pyridin-8-yl)ethan-1-ol. A mixture of 2-(2-(aminomethyl)-6-cyclopropylimidazo[1,2-a]pyridin-8-yl)ethan-1-ol (100 mg, crude from previous step), 2-bromo-4-fluoropyridine (150.0 mg, 0.86 mmol), and DIPEA (166.4 mg, 1.29 mmol) in i-PrOH (5 mL) was stirred at 100° C. for 16 h. The mixture was concentrated in vacuo and purified by preparative TLC (DCM/MeOH=15/1) to give 2-(2-(((2-bromopyridin-4-yl)amino)methyl)-6-cyclopropylimidazo[1,2-a]pyridin-8-yl)ethan-1-ol (80 mg, 48% over 2 steps) as yellow oil. ESI-MS [M+H]⁺: 387.2.

Synthesis of 2-bromo-N-((8-(2-chloroethyl)-6-cyclopropylimidazo[1,2-a]pyridin-2-yl)methyl)pyridin-4-amine. To a mixture of 2-(2-(((2-bromopyridin-4-yl)amino)methyl)-6-cyclopropylimidazo-[1,2-a]pyridin-8-yl)ethan-1-ol (600 mg, 1.55 mmol) in DCM (10.0 mL) was added SOCl₂ (1.0 mL) at 0° C. After stirring at 0° C. for 4 h, the mixture was concentrated in vacuo to give the crude 2-bromo-N-((8-(2-chloroethyl)-6-cyclopropylimidazo[1,2-a]pyridin-2-yl)methyl)pyridin-4-amine (600 mg, crude) as a yellow solid, which was used without further purification. ESI-MS [M+H]⁺: 405.2.

Synthesis of (1S,2S)—N-(4-(((8-(2-chloroethyl)-6-cyclopropylimidazo[1,2-a]pyridin-2-yl)-methyl)amino)pyridin-2-yl)-2-(3-chlorophenyl)cyclopropane-1-carboxamide. A mixture of 2-bromo-N-((8-(2-chloroethyl)-6-cyclopropylimidazo[1,2-a]pyridin-2-yl)-methyl)pyridin-4-amine (100.0 mg, 0.25 mmol), (1S,2S)-2-(3-chlorophenyl)cyclopropane-1-carboxamide (48 mg, 0.25 mmol), Pd₂(dba)₃ (23 mg, 0.025 mmol), Xantphos (14 mg, 0.025 mmol), and Cs₂CO₃ (202 mg, 0.62 mmol) in 1,4-dioxane (5.0 mL) was stirred at 100° C. under N₂ for 3 h. The mixture was concentrated in vacuo and purified by preparative TLC (DCM/MeOH=10/1) to give (1S,2S)—N-(4-(((8-(2-chloroethyl)-6-cyclopropylimidazo[1,2-a]pyridin-2-yl)methyl)amino)pyridin-2-yl)-2-(3-chlorophenyl)cyclopropane-1-carboxamide (50.0 mg, 39%) as a light yellow solid.

Synthesis of (1S,2S)-2-(3-chlorophenyl)-N-(4-(((6-cyclopropyl-8-(2-(dimethylamino)ethyl)imidazo-[1,2-a]pyridin-2-yl)methyl)amino)pyridin-2-yl)cyclopropane-1-carboxamide (I-598). A mixture of (1S,2S)—N-(4-(((8-(2-chloroethyl)-6-cyclopropylimidazo[1,2-a]pyridin-2-yl)-methyl)amino)pyridin-2-yl)-2-(3-chlorophenyl)cyclopropane-1-carboxamide (52 mg, 0.10 mmol) in MeNH₂ (2.0 M in THF, 3.0 mL) was stirred at 80° C. in a sealed tube for 48 h. The mixture was concentrated in vacuo and purified by preparative TLC (DCM/MeOH=10/1) to give (1S,2S)-2-(3-chlorophenyl)-N-(4-(((6-cyclopropyl-8-(2-(dimethylamino)ethyl)imidazo[1,2-a]pyridin-2-yl)methyl)amino)pyridin-2-yl)cyclopropane-1-carboxamide (6.0 mg, 11%) as a light-yellow solid. ESI-MS [M+H]+: 529.2, ¹H NMR (400 MHz, DMSO) δ 10.32 (s, 1H), 8.16 (s, 1H), 7.75 (d, J=5.7 Hz, 1H), 7.60 (s, 1H), 7.44 (s, 1H), 7.37-7.20 (m, 3H), 7.14 (d, J=6.1 Hz, 2H), 6.84 (s, 1H), 6.34-6.32 (m, 1H), 4.36 (d, J=5.7 Hz, 2H), 3.00 (t, J=7.4 Hz, 2H), 2.79-2.63 (m, 2H), 2.41-2.31 (m, 2H), 2.26 (s, 6H), 1.93-1.79 (m, 1H), 1.54-1.41 (m, 1H), 1.37-1.32 (m, 1H), 0.98-0.79 (m, 2H), 0.76-0.52 (m, 2H).

### Example 599

### Synthesis of (1S,2S)-2-(3-chlorophenyl)-N-(4-(((6-cyclopropyl-8-(4-(2-methoxyethyl)piperazin-1-yl)imidazo[1,2-a]pyridin-2-yl)methyl)amino)pyridin-2-yl)cyclopropane-1-carboxamide (I-599)

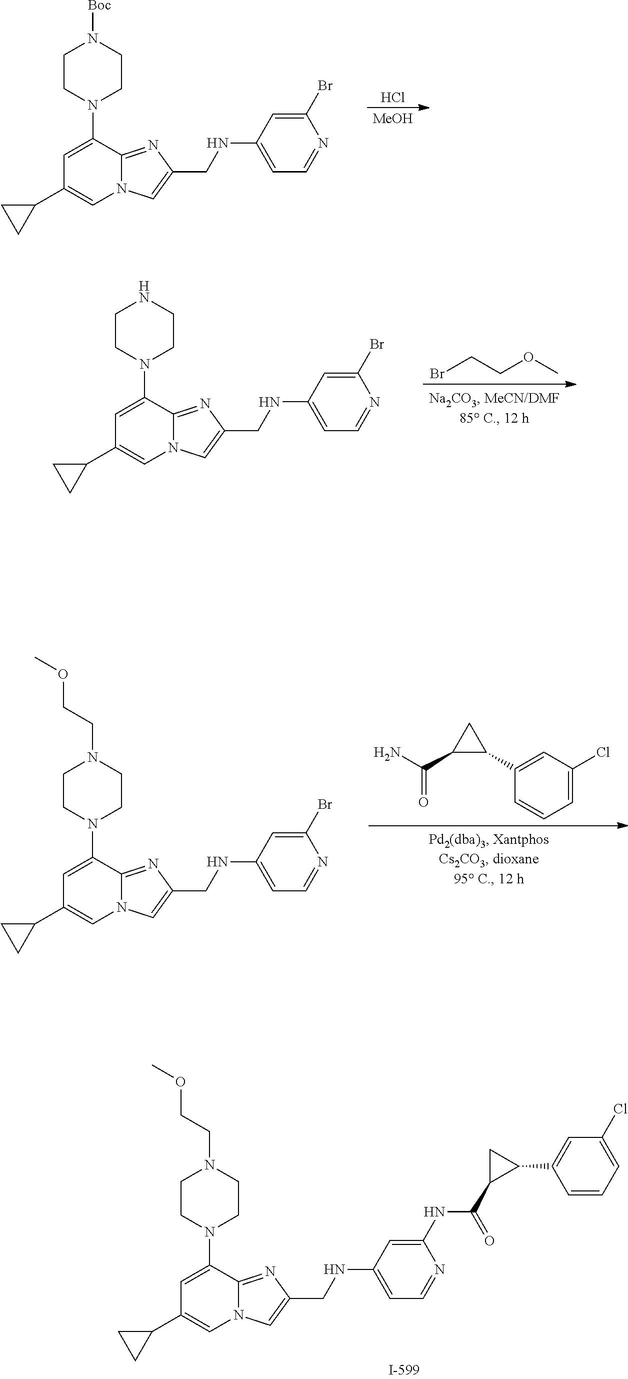

Synthesis of 2-bromo-N-((6-cyclopropyl-8-(piperazin-1-yl)imidazo[1,2-a]pyridin-2-yl)methyl)pyridin-4-amine. To a solution of tert-butyl 4-(2-(((2-bromopyridin-4-yl)amino)methyl)-6-cyclopropylimidazo[1,2-a]pyridin-8-yl)piperazine-1-carboxylate (500 mg, 0.95 mmol) in MeOH (10 ml) was added HCl (4 M solution in MeOH, 1.19 ml, 4.75 mmol). After stirring at 0° C. for 5 h, the mixture was concentrated in vacuo to give the 2-bromo-N-((6-cyclopropyl-8-(piperazin-1-yl)imidazo[1,2-a]pyridin-2-yl)methyl)pyridin-4-amine as a light-yellow oil (300 mg, crude). ESI-MS [M+H]+: 427.2

Synthesis of 2-bromo-N-((6-cyclopropyl-8-(4-(2-methoxyethyl)piperazin-1-yl)imidazo[1,2-a]pyridin-2-yl)methyl)pyridin-4-amine. To a solution of 2-bromo-N-((6-cyclopropyl-8-(piperazin-1-yl)imidazo[1,2-a]pyridin-2-yl)methyl)pyridin-4-amine (50 mg, 0.12 mmol) and Na₂CO₃ (38 mg, 0.36 mmol) in MeCN/DMF (10 mL/1 mL), 1-bromo-2-methoxyethane (13 mg, 0.096 mmol) was added. After stirring at 85° C. for 12 h, the reaction was quenched with water (30 mL) and extracted with EtOAc (3×30 mL). The combined organic layers were washed with brine (50 mL), dried over Na₂SO₄ then concentrated in vacuo to give the crude, which was purified by preparative TLC (DCM/MeOH=10/1) to give 2-bromo-N-((6-cyclopropyl-8-(4-(2-methoxyethyl)piperazin-1-yl)imidazo[1,2-a]pyridin-2-yl)methyl)pyridin-4-amine (30 mg, 52%) as a white solid. ESI-MS [M+H]+: 485.2

Synthesis of (1S,2S)-2-(3-chlorophenyl)-N-(4-(((6-cyclopropyl-8-(4-(2-methoxyethyl)piperazin-1-yl)imidazo[1,2-a]pyridin-2-yl)methyl)amino)pyridin-2-yl)cyclopropane-1-carboxamide (I-599). To a solution of 2-bromo-N-((6-cyclopropyl-8-(4-(2-methoxyethyl)piperazin-1-yl)imidazo[1,2-a]pyridin-2-yl)methyl)pyridin-4-amine (30 mg, 0.062 mmol), (1S,2S)-2-(3-chlorophenyl)cyclopropane-1-carboxamide (18 mg, 0.092 mmol), Pd₂(dba)₃ (6 mg, 0.0062 mmol), Xantphos (4 mg, 0.0062 mmol), and Cs₂CO₃ (61 mg, 0.186 mmol) in 1,4-dioxane (10 mL) was stirred at 95° C. for 12 h under N₂. After quenching with water (20 mL), the aqueous phase was extracted with EtOAc (3×20 mL). The combined organic layers were washed with brine (50 mL), dried over Na₂SO₄ then concentrated in vacuo to give the crude, which was purified by preparative TLC (DCM/MeOH=10/1) to give (1S,2S)-2-(3-chlorophenyl)-N-(4-(((6-cyclopropyl-8-(4-(2-methoxyethyl)piperazin-1-yl)imidazo[1,2-a]pyridin-2-yl)methyl)amino)pyridin-2-yl)cyclopropane-1-carboxamide (20 mg, 54%) as a white solid. ESI-MS [M+H]+: 600.3. ¹H NMR (400 MHz, DMSO) δ 10.32 (s, 1H), 7.87 (s, 1H), 7.75 (d, J=5.8 Hz, 1H), 7.53 (s, 1H), 7.44 (s, 1H), 7.33-7.20 (m, 3H), 7.19-7.10 (m, 2H), 6.32 (dd, J=5.8, 2.1 Hz, 1H), 6.14 (s, 1H), 4.33 (d, J=5.7 Hz, 2H), 3.49-3.46 (m, 6H), 3.25 (s, 3H), 2.67-2.55 (m, J=25.8 Hz, 4H), 2.54-2.51 (m, 2H), 2.40-2.33 (m, 2H), 1.88-1.81 (m, J=13.4, 8.4, 5.1 Hz, 1H), 1.46-1.42 (m, 1H), 1.37-1.32 (m, 1H), 0.88-0.83 (m, 2H), 0.67-0.63 (m, 2H).

### Example 600

### Synthesis of ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (I-600)

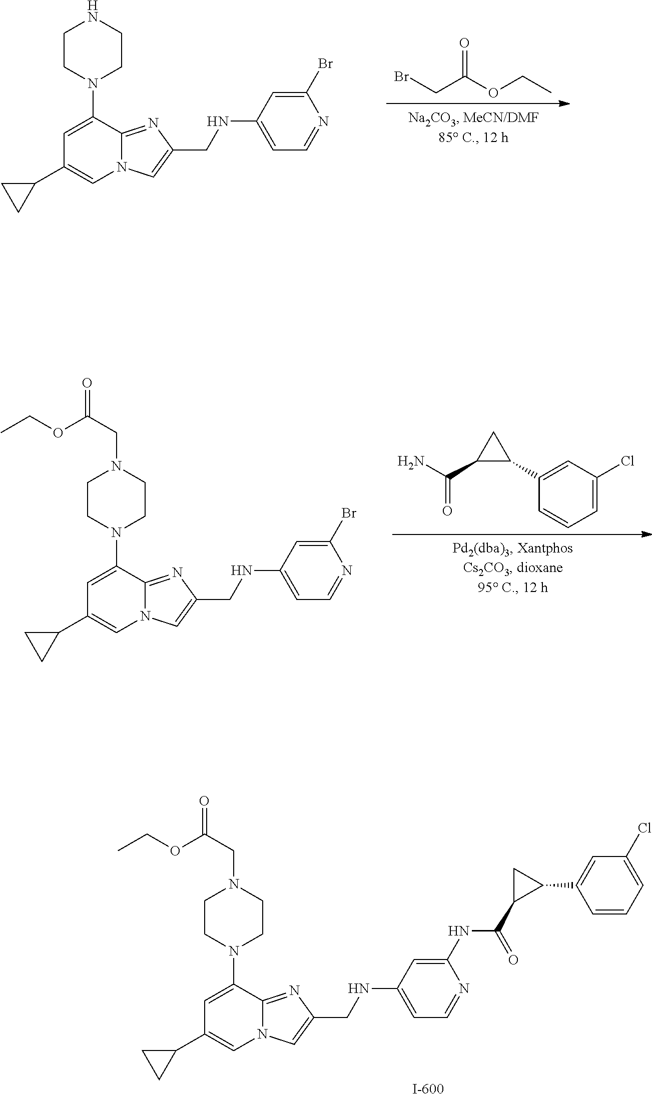

Synthesis of ethyl 2-(4-(2-(((2-bromopyridin-4-yl)amino)methyl)-6-cyclopropylimidazo[1,2-a]pyridin-8-yl)piperazin-1-yl)acetate. To a solution of 2-bromo-N-((6-cyclopropyl-8-(piperazin-1-yl)imidazo[1,2-a]pyridin-2-yl)methyl)pyridin-4-amine (90 mg, 0.21 mmol) and Na2CO3 (67 mg, 0.63 mmol) in MeCN/DMF (10 mL/1 mL), ethyl 2-bromoacetate (35 mg, 0.21 mmol) was added. After stirring at 85° C. for 12 h, the reaction was quenched with water (30 mL) and extracted with EtOAc (3×20 mL). The combined organic layers were washed with brine (50 mL), dried over Na2SO4, and concentrated in vacuo to give the crude, which was purified by preparative TLC (DCM/MeOH=10/1) to give the product ethyl 2-(4-(2-(((2-bromopyridin-4-yl)amino)methyl)-6-cyclopropylimidazo[1,2-a]pyridin-8-yl)piperazin-1-yl)acetate (100 mg, 90%) as a white solid. ESI-MS [M+H]+: 513.2

Synthesis of ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (I-600). A solution of ethyl 2-(4-(2-(((2-bromopyridin-4-yl)amino)methyl)-6-cyclopropylimidazo[1,2-a]pyridin-8-yl)piperazin-1-yl)acetate (100 mg, 0.19 mmol), (1S,2S)-2-(3-chlorophenyl)cyclopropane-1-carboxamide (57 mg, 0.29 mmol), Pd₂(dba)₃ (17 mg, 0.019 mmol), Xantphos (11 mg, 0.019 mmol), and Cs₂CO₃ (186 mg, 0.57 mmol) in 1,4-dioxane (10 mL) was stirred at 95° C. for 12 h under N₂. The reaction was quenched with water (20 mL) and the aqueous phase was extracted with EtOAc (3×20 mL). The combined organic layers were washed with brine (50 mL), dried over Na₂SO₄ then concentrated in vacuo to give the crude, which was purified by preparative TLC (DCM/MeOH=10/1) to give the product ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (90 mg, 75%) as a white solid.

ESI-MS [M+H]+: 628.4, ¹H NMR (400 MHz, DMSO) δ 10.32 (s, 1H), 7.87 (s, 1H), 7.80-7.71 (m, 1H), 7.53 (s, 1H), 7.43 (s, 1H), 7.36-7.07 (m, 5H), 6.33 (d, J=5.2 Hz, 1H), 6.16 (s, 1H), 4.39-4.29 (m, 2H), 4.20-4.06 (m, 2H), 3.52-3.46 (m, 4H), 2.76-2.68 (m, 4H), 2.41-2.29 (m, 2H), 1.87-1.82 (m, 1H), 1.47-1.42 (m, 1H), 1.37-1.32 (m, 1H), 1.24-1.17 (m, 3H), 0.89-0.83 (m, 2H), 0.69-0.60 (m, 2H).

### Example 601

### Synthesis of (1S,2S)-2-(3-chlorophenyl)-N-(4-(((6-cyclopropyl-8-(4-(cyclopropylmethyl)piperazin-1-yl)imidazo[1,2-a]pyridin-2-yl)methyl)amino)pyridin-2-yl)cyclopropane-1-carboxamide (I-601)

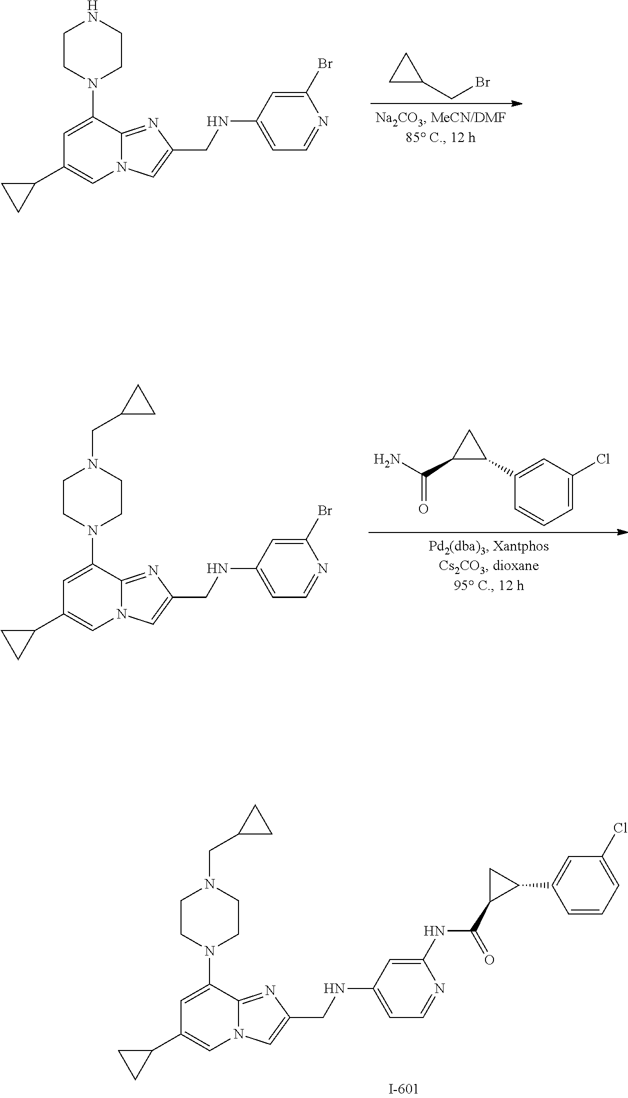 [SMILES: Brc1cc(NCc2cn3cc(C4CC4)cc(N4CCNCC4)c3n2)ccn1 ; BrCC1CC1 ; Brc1cc(NCc2cn3cc(C4CC4)cc(N4CCN(CC5CC5)CC4)c3n2)ccn1 ; NC(=O)[C@H]1C[C@@H]1c1cccc(Cl)c1 ; O=C(Nc1cc(NCc2cn3cc(C4CC4)cc(N4CCN(CC5CC5)CC4)c3n2)ccn1)[C@H]1C[C@@H]1c1cccc(Cl)c1]

Synthesis of 2-bromo-N-((6-cyclopropyl-8-(4-(cyclopropylmethyl)piperazin-1-yl)imidazo[1,2-a]pyridin-2-yl)methyl)pyridin-4-amine. To a solution of 2-bromo-N-((6-cyclopropyl-8-(piperazin-1-yl)imidazo[1,2-a]pyridin-2-yl)methyl)pyridin-4-amine (80 mg, 0.19 mmol) and Na₂CO₃ (60 mg, 0.56 mmol) in MeCN/DMF (15 mL/1 mL) was added (bromomethyl)cyclopropane (25 mg, 0.19 mmol). After stirring at 85° C. for 12 h, the reaction was quenched with water (30 mL) and extracted with EtOAc (3×30 mL). The combined organic layers were washed with brine (30 mL), dried over Na₂SO₄ then concentrated in vacuo to give the crude, which was purified by Pre-TLC (eluent: DCM/MeOH=10/1) to give 2-bromo-N-((6-cyclopropyl-8-(4-(cyclopropylmethyl)piperazin-1-yl)imidazo[1,2-a]pyridin-2-yl)methyl)pyridin-4-amine (60 mg, 66%) as a white solid. ESI-MS [M+H]+: 481.1

Synthesis of (1S,2S)-2-(3-chlorophenyl)-N-(4-(((6-cyclopropyl-8-(4-(cyclopropylmethyl)piperazin-1-yl)imidazo[1,2-a]pyridin-2-yl)methyl)amino)pyridin-2-yl)cyclopropane-1-carboxamide (I-601). A mixture of 2-bromo-N-((6-cyclopropyl-8-(4-(cyclopropylmethyl)piperazin-1-yl)imidazo[1,2-a]pyridin-2-yl)methyl)pyridin-4-amine (60 mg, 0.13 mmol), (1S,2S)-2-(3-chlorophenyl)cyclopropane-1-carboxamide (27 mg, 0.13 mmol), Pd₂(dba)₃ (23 mg, 0.025 mmol), Xantphos (15 mg, 0.025 mmol), and Cs₂CO₃ (122 mg, 0.38 mmol) in 1,4-dioxane (15 mL) was stirred at 95° C. for 12 h. The reaction was quenched with water (30 mL) and extracted with EtOAc (3×30 mL). The combined organic layers were washed with brine (50 mL), dried over Na₂SO₄ then concentrated in vacuo to give the crude, which was purified by Pre-TLC (eluent: DCM/MeOH=10/1) to give (1S,2S)-2-(3-chlorophenyl)-N-(4-(((6-cyclopropyl-8-(4-(cyclopropylmethyl)piperazin-1-yl)imidazo[1,2-a]pyridin-2-yl)methyl)amino)pyridin-2-yl)cyclopropane-1-carboxamide (20 mg, 27%) as a white solid. ESI-MS [M+H]+: 596.3, ¹H NMR (400 MHz, DMSO) δ 10.31 (s, 1H), 7.86 (s, 1H), 7.75 (d, J=5.8 Hz, 1H), 7.53 (s, 1H), 7.44 (s, 1H), 7.35-7.28 (m, 1H), 7.27-7.22 (m, 2H), 7.16-7.08 (m, 2H), 6.32 (dd, J=5.8, 2.1 Hz, 1H), 6.16 (s, 1H), 4.33 (d, J=5.5 Hz, 2H), 3.49 (s, 4H), 2.64 (s, 4H), 2.43-2.30 (m, 2H), 2.25 (d, J=6.4 Hz, 2H), 1.92-1.80 (m, 1H), 1.49-1.40 (m, 1H), 1.39-1.30 (m, 1H), 0.90-0.82 (m, 3H), 0.70-0.62 (m, 2H), 0.52-0.45 (m, 2H), 0.15-0.08 (m, 2H).

### Example 602

### Synthesis of 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetic acid (I-602)

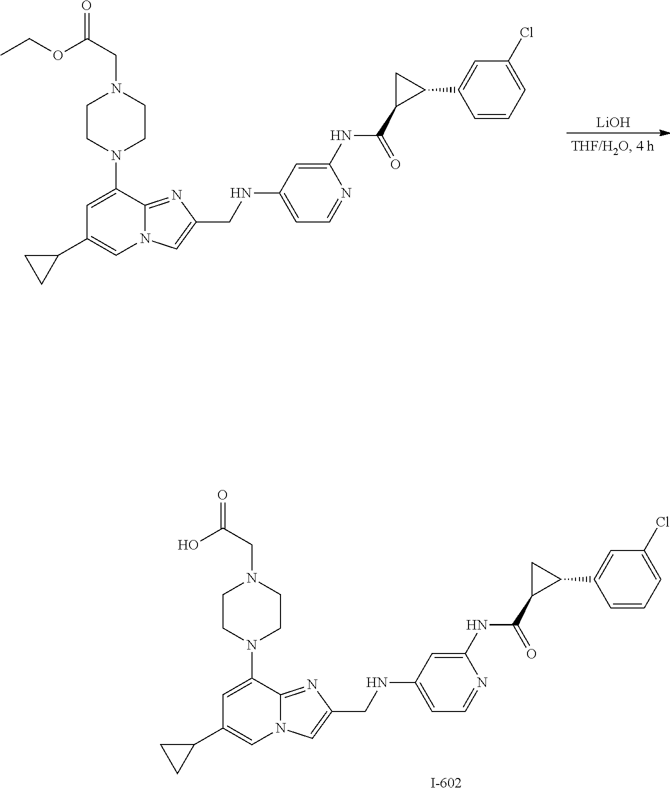 [SMILES: CCOC(=O)CN1CCN(c2cc(C3CC3)cn3cc(CNc4ccnc(NC(=O)[C@H]5C[C@@H]5c5cccc(Cl)c5)c4)nc23)CC1 ; "LiOH" ; O=C(O)CN1CCN(c2cc(C3CC3)cn3cc(CNc4ccnc(NC(=O)[C@H]5C[C@@H]5c5cccc(Cl)c5)c4)nc23)CC1]

To a solution of ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (30 mg, 0.048 mmol) in THF (3 mL) and water (3 mL) was added LiOH—H₂O (4 mg, 0.095 mmol). The mixture was stirred at room temperature for 4 h. The organic layer was evaporated, and the aqueous phase was acidified with 1 N HCl to pH=3 and extracted with EtOAc (3×10 mL). The combined organic layers were washed with brine (10 mL), dried over Na₂SO₄ and concentrated in vacuo. The residue was purified by column chromatography (eluent: DCM/MeOH=7/1) to give 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetic acid (12 mg, 42% yield). ESI-MS [M+H]⁺: 600.2, ¹H NMR (400 MHz, DMSO) δ 10.44 (s, 1H), 7.89 (s, 1H), 7.76 (d, J=5.8 Hz, 1H), 7.55 (s, 1H), 7.39 (s, 1H), 7.34-7.21 (m, 4H), 7.14 (d, J=7.7 Hz, 1H), 6.36 (d, J=4.9 Hz, 1H), 6.19 (s, 1H), 4.34 (d, J=5.5 Hz, 2H), 3.54 (s, 4H), 3.26 (s, 2H), 2.83 (s, 4H), 2.39-2.32 (m, 2H), 1.88-1.82 (m, 1H), 1.50-1.42 (m, 1H), 1.38-1.32 (m, 1H), 0.88-0.82 (m, 2H), 0.68-0.62 (m, 2H).

### Example 603

### Synthesis of (1S,2S)-2-(3-chlorophenyl)-N-(4-(((6-cyclopropyl-8-(4-(2-hydroxyethyl)piperazin-1-yl)imidazo[1,2-a]pyridin-2-yl)methyl)amino)pyridin-2-yl)cyclopropane-1-carboxamide (I-603)

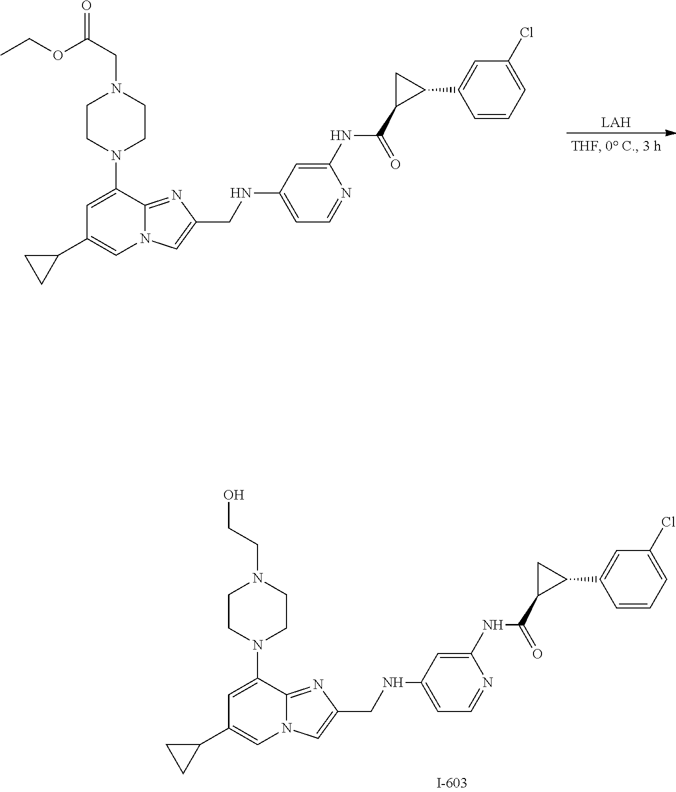 [SMILES: CCOC(=O)CN1CCN(c2cc(C3CC3)cn3cc(CNc4ccnc(NC(=O)[C@H]5C[C@@H]5c5cccc(Cl)c5)c4)nc23)CC1 ; "LAH" ; O=C(Nc1cc(NCc2cn3cc(C4CC4)cc(N4CCN(CCO)CC4)c3n2)ccn1)[C@H]1C[C@@H]1c1cccc(Cl)c1]

To a solution of ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate (15 mg, 0.024 mmol) in THF (5 mL) was added LAH (2 mg, 0.048 mmol) at 0° C. After stirring at 0° C. for 3 h, the reaction was quenched with water (15 mL) and extracted with EtOAc (3×10 mL). The combined organic layers were washed with brine (15 mL), dried over Na₂SO₄ then concentrated in vacuo to give the crude, which was purified by column chromatography (eluent: DCM/MeOH=10/1) to give the (1S,2S)-2-(3-chlorophenyl)-N-(4-(((6-cyclopropyl-8-(4-(2-hydroxyethyl)piperazin-1-yl)imidazo[1,2-a]pyridin-2-yl)methyl)amino)pyridin-2-yl)cyclopropane-1-carboxamide as a white solid. (10 mg, 69% yield). ESI-MS [M+H]⁺: 586.3, ¹H NMR (400 MHz, DMSO) δ 10.31 (s, 1H), 7.87 (s, 1H), 7.75 (d, J=5.8 Hz, 1H), 7.53 (s, 1H), 7.44 (s, 1H), 7.34-7.22 (m, 3H), 7.17-7.04 (m, 2H), 6.33-6.31 (m, 1H), 6.15 (s, 1H), 4.44 (s, 1H), 4.33 (d, J=5.6 Hz, 2H), 3.56-3.47 (m, 6H), 2.68-2.62 (m, 6H), 2.38-2.34 (m, 2H), 1.89-1.78 (m, 1H), 1.48-1.40 (m, 1H), 1.38-1.32 (m, 1H), 0.88-0.82 (m, 2H), 0.71-0.61 (m, 2H).

### Example 604

### Synthesis of (1S,2S)-2-(3-chlorophenyl)-N-(4-(((6-cyclopropylimidazo[1,2-a]pyridin-2-yl)methyl)(2-hydroxyethyl)amino)pyridin-2-yl)cyclopropane-1-carboxamide (I-604)

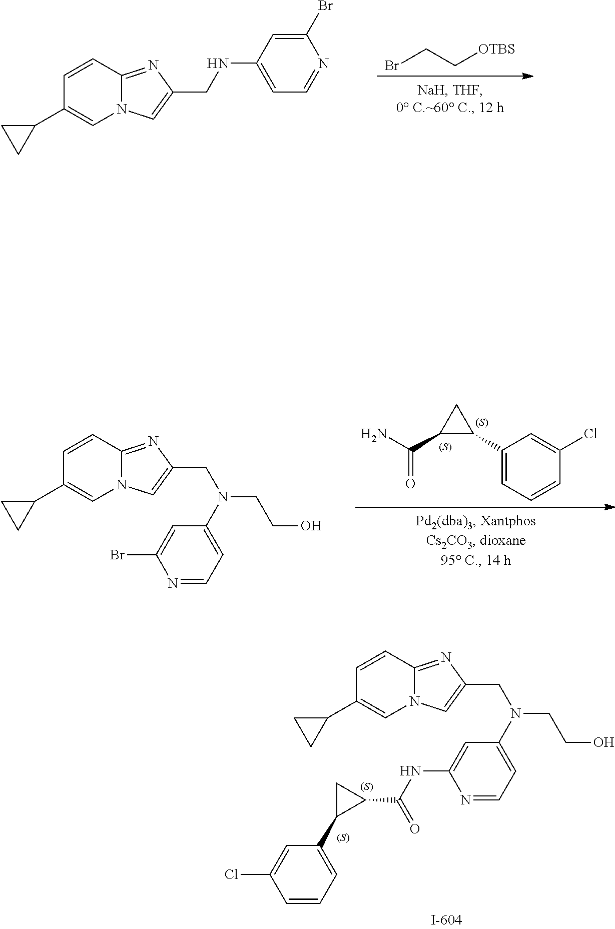

Synthesis of 2-((2-bromopyridin-4-yl)((6-cyclopropylimidazo[1,2-a]pyridin-2-yl)methyl)amino)ethan-1-ol. To a mixture of 2-bromo-N-((6-cyclopropylimidazo[1,2-a]pyridin-2-yl)methyl)pyridin-4-amine (300 mg, 0.88 mmol) in DMF (5 mL) was added NaH (126 mg, 5.26 mmol) at 0° C. After stirring at room temperature for 1 h, (2-bromoethoxy)(tert-butyl)dimethylsilane (315 mg, 1.32 mmol) was added to the mixture and stirred at 60° C. for 12 h. Water (30 mL) was added and the mixture was extracted with EtOAc (3×30 mL). The combined organic layers were washed with brine (20 mL), dried over Na₂SO₄ and concentrated in vacuo to give the crude, which was purified by column chromatography (eluent: DCM/MeOH=20/1) to 2-((2-bromopyridin-4-yl)((6-cyclopropylimidazo[1,2-a]pyridin-2-yl)methyl)amino)ethan-1-ol (100 mg, 28%) as a yellow solid. ESI-MS [M+H]+: 387.2.

Synthesis of (1S,2S)-2-(3-chlorophenyl)-N-(4-(((6-cyclopropylimidazo[1,2-a]pyridin-2-yl)methyl)(2-hydroxyethyl)amino)pyridin-2-yl)cyclopropane-1-carboxamide (I-604). To a mixture of 6-bromo-N-((5-cyclopropyl-7-(4-methylpiperazin-1-yl)pyrazolo[1,5-a]pyridin-2-yl)methyl)pyrimidin-4-amine (50 mg, 0.13 mmol), (1S,2S)-2-(3-chlorophenyl)cyclopropane-carboxamide (25 mg, 0.13 mmol), and Cs₂CO₃ (126 mg, 0.39 mmol) in 1,4-dioxane (5 mL) were added Pd₂(dba)₃ (17 mg, 0.019 mmol) and Xantphos (15.0 mg, 0.026 mmol). The mixture was stirred at 95° C. for 14 h under N₂ and then filtered through Celite®. The filter cake was washed with DCM/MeOH (10/1, 30 mL) and the filtrate was concentrated in vacuo to give the crude, which was purified by preparative TLC (DCM/MeOH=20/1) to give (1S,2S)-2-(3-chlorophenyl)-N-(4-(((6-cyclopropylimidazo[1,2-a]pyridin-2-yl)methyl)(2-hydroxyethyl)amino)pyridin-2-yl)cyclopropane-1-carboxamide (30 mg, 47%) as a yellow solid. ESI-MS [M+H]+: 502.2, ¹H NMR (400 MHz, DMSO) δ 10.38 (s, 1H), 8.30 (s, 1H), 7.79 (d, J=6.0 Hz, 1H), 7.65 (s, 1H), 7.57 (s, 1H), 7.38 (d, J=9.3 Hz, 1H), 7.26-7.25 (m, 1H), 7.24 (d, J=7.1 Hz, 2H), 7.14 (d, J=7.6 Hz, 1H), 7.00-6.97 (m, 1H), 6.46-6.45 (m, 1H), 5.33 (s, 1H), 4.66 (s, 2H), 3.66-3.51 (m, 4H), 2.42-2.33 (m, 2H), 1.93-1.89 (m, 1H), 1.47-1.41 (m, 1H), 1.38-1.33 (m, 1H), 0.91-1.85 (m, 2H), 0.67-0.64 (m, 2H).

### Example 605

### Synthesis of (1S,2S)-2-(3-chlorophenyl)-N-(4-(((6-cyclopropyl-8-(4-methylpiperazin-1-yl)imidazo[1,2-a]pyridin-2-yl)methyl)amino)-6-methylpyridin-2-yl)cyclopropane-1-carboxamide (I-605)

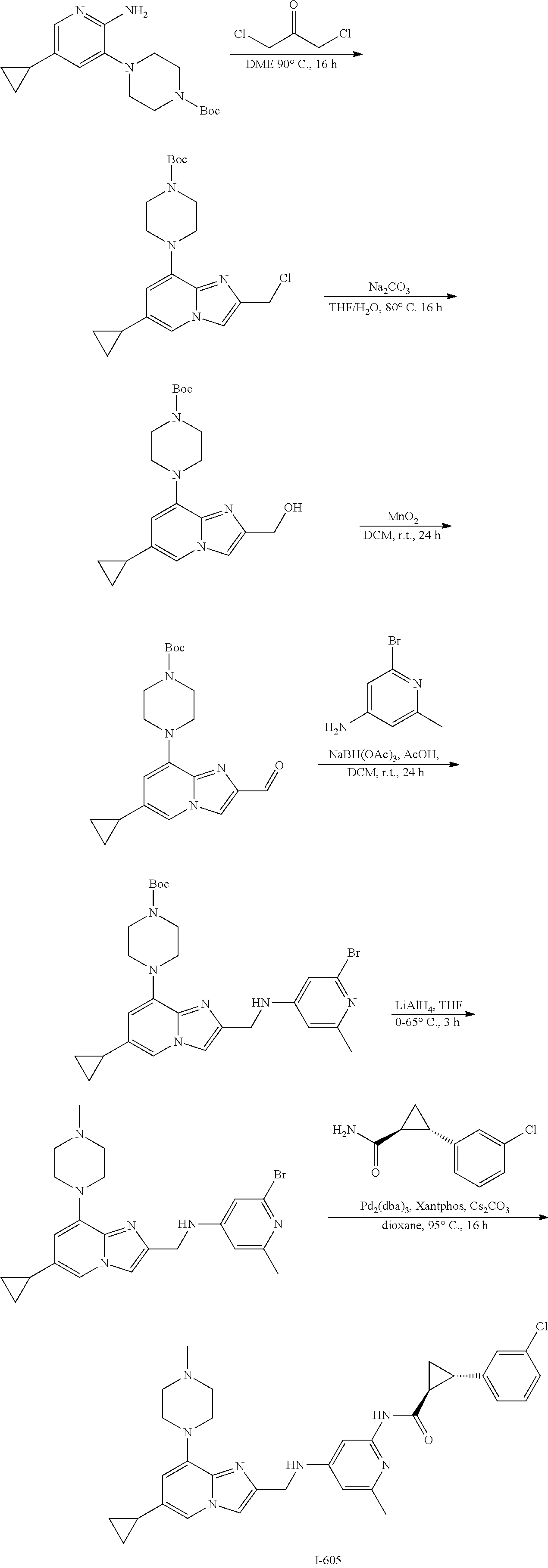

Synthesis of tert-butyl 4-(2-(chloromethyl)-6-cyclopropylimidazo[1,2-a]pyridin-8-yl)piperazine-1-carboxylate. A mixture of tert-butyl 4-(2-amino-5-cyclopropylpyridin-3-yl)piperazine-1-carboxylate (800 mg, 2.52 mmol), 1,3-dichloropropan-2-one (639 mg, 5.03 mmol), and DIPEA (649 mg, 5.03 mmol) in DME (10 mL) was stirred at 90° C. for 16 h. The mixture was quenched with saturated aq·NaHCO₃ (50 mL) and extracted with EtOAc (3×50 mL). The combined organic layers were washed with brine (50 mL), dried over Na₂SO₄ and concentrated in vacuo to give tert-butyl 4-(2-(chloromethyl)-6-cyclopropylimidazo[1,2-a]pyridin-8-yl)piperazine-1-carboxylate as a yellow solid (700 mg, crude), which was used without purification. ESI-MS [M+H]+: 391.2.

Synthesis of tert-butyl 4-(6-cyclopropyl-2-(hydroxymethyl)imidazo[1,2-a]pyridin-8-yl)piperazine-1-carboxylate. A mixture of tert-butyl 4-(2-(chloromethyl)-6-cyclopropylimidazo[1,2-a]pyridin-8-yl)-piperazine-1-carboxylate (700 mg, 1.79 mmol, crude) and Na₂CO₃ (951 mg, 8.97 mmol) in THF/H₂O (45 mL/45 mL) was stirred at 80° C. for 16 h. The mixture was cooled to room temperature and extracted with EtOAc (3×50 mL). The organic layers were washed with brine (50 mL), dried over Na₂SO₄ and concentrated in vacuo. The residue was purified by column chromatography (eluent: DCM/MeOH=0˜15%) to give tert-butyl 4-(6-cyclopropyl-2-(hydroxymethyl)imidazo[1,2-a]pyridin-8-yl)piperazine-1-carboxylate as a red solid (470 mg, 70%). ESI-MS [M+H]⁺: 373.2.

Synthesis of tert-butyl 4-(6-cyclopropyl-2-formylimidazo[1,2-a]pyridin-8-yl)piperazine-1-carboxylate. A mixture of tert-butyl 4-(6-cyclopropyl-2-(hydroxymethyl)imidazo[1,2-a]pyridin-8-yl)-piperazine-1-carboxylate (370 mg, 1.0 mmol) and MnO₂ (435 mg, 5.0 mmol) in DCM (10 mL) was stirred at room temperature for 16 h. Further MnO₂ (435 mg, 5.0 mmol) was added into the mixture and stirring was continued for 8 h. The mixture was filtered and the filtrate was concentrated in vacuo to give the tert-butyl 4-(6-cyclopropyl-2-formylimidazo[1,2-a]pyridin-8-yl)-piperazine-1-carboxylate as a yellow solid (170 mg, crude) which was used without purification. ESI-MS [M+H]⁺: 371.2

Synthesis of tert-butyl 4-(2-(((2-bromo-6-methylpyridin-4-yl)amino)methyl)-6-cyclopropylimidazo-[1,2-a]pyridin-8-yl)piperazine-1-carboxylate. A mixture of tert-butyl 4-(6-cyclopropyl-2-formylimidazo[1,2-a]pyridin-8-yl)piperazine-1-carboxylate (370 mg, 1.0 mmol), 2-bromo-6-methylpyridin-4-amine (186 mg, 1.0 mmol), and AcOH (2 drops) in DCM (5 mL) was stirred at room temperature for 8 h. Then NaBH(OAc)₃ (318 mg, 1.5 mmol) was added and the mixture was stirred at room temperature for 16 h. The mixture was diluted with water (15 mL) and extracted with EtOAc (3×15 mL). The organic layers were washed with brine (15 mL), dried over Na₂SO₄ and concentrated in vacuo. The residue was purified by silica gel column chromatography (eluent: DCM/MeOH=20:1) to give tert-butyl 4-(2-(((2-bromo-6-methylpyridin-4-yl)amino)methyl)-6-cyclopropylimidazo[1,2-a]pyridin-8-yl)piperazine-1-carboxylates as a yellow solid (75 mg, 20%). ESI-MS [M+H]⁺: 541.3.

Synthesis of 2-bromo-N-((6-cyclopropyl-8-(4-methylpiperazin-1-yl)imidazo[1,2-a]pyridin-2-yl)methyl)-6-methylpyridin-4-amine. To a mixture of tert-butyl 4-(2-(((2-bromo-6-methylpyridin-4-yl)amino)methyl)-6-cyclopropylimidazo[1,2-a]pyridin-8-yl)piperazine-1-carboxylate (60 mg, 0.11 mmol) in THF (2 mL) was added LiAlH₄ (0.33 mL, 0.33 mmol) at 0° C. under N₂. The mixture was stirred at 65° C. for 16 h under N₂. The reaction was quenched with saturated aq·KOH (5 mL) and extracted with DCM (3×15 mL). The organic layers were washed with brine (15 mL), dried over Na₂SO₄ and concentrated in vacuo. The residue was purified by preparative TLC (eluent: DCM/MeOH=20/1) to give 2-bromo-N-((6-cyclopropyl-8-(4-methylpiperazin-1-yl)imidazo[1,2-a]pyridin-2-yl)methyl)-6-methylpyridin-4-amine as a yellow solid (30 mg, 59%). ESI-MS [M+H]⁺: 455.1.

Synthesis of (1S,2S)-2-(3-chlorophenyl)-N-(4-(((6-cyclopropyl-8-(4-methylpiperazin-1-yl)imidazo[1,2-a]pyridin-2-yl)methyl)amino)-6-methylpyridin-2-yl)cyclopropane-1-carboxamide (I-605). A mixture of 2-bromo-N-((6-cyclopropyl-8-(4-methylpiperazin-1-yl)imidazo-[1,2-a]pyridin-2-yl)methyl)-6-methylpyridin-4-amine (30 mg, 0.066 mmol), (1S,2S)-2-(3-chlorophenyl)cyclopropane-1-carboxamide (13 mg, 0.066 mmol), Pd₂(dba)₃ (6 mg, 0.0066 mmol), Xantphos (4 mg, 0.0066 mmol), and Cs₂CO₃ (65 mg, 0.2 mmol) in dioxane (3 mL) was stirred at 95° C. for 16 h under N₂. The mixture was filtered through Celite® and the filter cake was washed with DCM/MeOH (10/1, 30 mL). The filtrate was concentrated in vacuo to give the crude, which was purified by preparative TLC (DCM/MeOH=10/1) to give (1S,2S)-2-(3-chlorophenyl)-N-(4-(((6-cyclopropyl-8-(4-methylpiperazin-1-yl)imidazo[1,2-a]pyridin-2-yl)methyl)amino)-6-methylpyridin-2-yl)cyclopropane-1-carboxamide as a light yellow solid (16.6 mg, 45%). ESI-MS [M+H]+: 570.2. ¹H NMR (400 MHz, DMSO) δ 10.30 (s, 1H), 7.86 (s, 1H), 7.50 (s, 1H), 7.31-7.26 (m, 2H), 7.24-7.22 (m, 1H), 7.21-7.21 (m, 1H), 7.11-7.09 (m, 1H), 6.99-6.96 (m, 1H), 6.20 (d, J=1.6 Hz, 1H), 6.15 (d, 1H), 4.30 (d, J=5.8 Hz, 2H), 3.50 (s, 4H), 2.66-2.65 (m, 3H), 2.33-2.31 (m, 4H), 2.16 (s, 3H), 1.86-1.79 (m, 2H), 1.41-1.39 (m, 1H), 1.31-1.28 (m, 1H), 1.23-1.20 (m, 1H), 0.85-0.83 (m, 2H), 0.65-0.63 (m, 2H).

### Example 606

### Synthesis of (1S,2S)-2-(3-chlorophenyl)-N-(5-(((6-cyclopropylimidazo[1,2-a]pyridin-2-yl)methyl)amino)-1,3,4-thiadiazol-2-yl)cyclopropane-1-carboxamide (I-606)

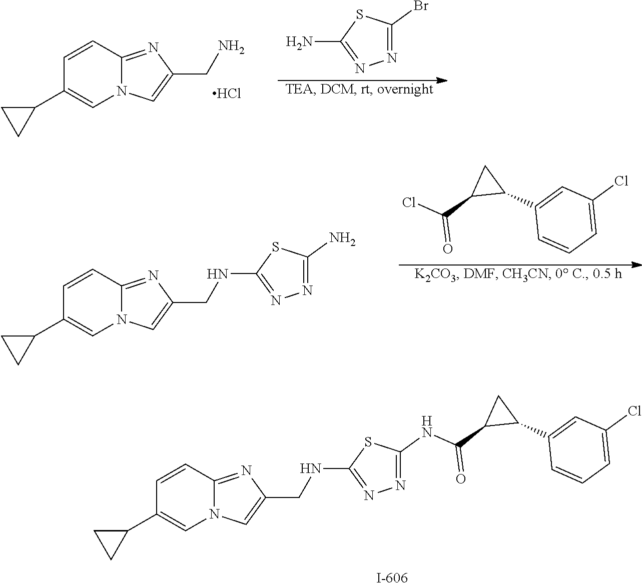 [SMILES: NCc1cn2cc(C3CC3)ccc2n1 ; Cl ; Nc1nnc(Br)s1 ; Nc1nnc(NCc2cn3cc(C4CC4)ccc3n2)s1 ; O=C(Cl)[C@H]1C[C@@H]1c1cccc(Cl)c1 ; O=C(Nc1nnc(NCc2cn3cc(C4CC4)ccc3n2)s1)[C@H]1C[C@@H]1c1cccc(Cl)c1]

Synthesis of N²-((6-cyclopropylimidazo[1,2-a]pyridin-2-yl)methyl)-1,3,4-thiadiazole-2,5-diamine. To a solution of (6-cyclopropylimidazo[1,2-a]pyridin-2-yl)methanamine hydrochloride (600 mg, 2.68 mmol) in DCM (7 mL) was added TEA (1.12 mL, 8.05 mmol) and 5-bromo-1,3,4-thiadiazol-2-amine (724 mg, 4.02 mmol). After stirring at room temperature overnight, the reaction was quenched with saturated aq·NaHCO₃ (50 mL) and extracted with EtOAc (3×50 mL). The combined organic layers were washed with brine (30 mL), dried over Na₂SO₄ and concentrated in vacuo. The residue was purified by silica gel column chromatography (eluent: MeOH/DCM=0-10%) to afford N²-((6-cyclopropylimidazo[1,2-a]pyridin-2-yl)methyl)-1,3,4-thiadiazole-2,5-diamine (206 mg, yield 27%) as off-white solid. ESI-MS [M+H]+: 287.1.

Synthesis of (1S,2S)-2-(3-chlorophenyl)-N-(5-(((6-cyclopropylimidazo[1,2-a]pyridin-2-yl)methyl)amino)-1,3,4-thiadiazol-2-yl)cyclopropane-1-carboxamide (I-606). To a solution of N2-((6-cyclopropylimidazo[1,2-a]pyridin-2-yl)methyl)-1,3,4-thiadiazole-2,5-diamine (100 mg, 0.349 mmol) in DMF (0.7 mL) and CH₃CN (0.7 mL) was added K₂CO₃ (145 mg, 1.05 mmol). A solution of (1S,2S)-2-(3-chlorophenyl)cyclopropane-1-carbonyl chloride (80 mg, 0.347 mmol) in DCM (0.5 mL) was then added at 0° C. and the mixture stirred at 0° C. for 0.5 h. The mixture was diluted with water (30 mL) and extracted with DCM (3×20 mL). The combined organic layers were washed with brine (20 mL), dried over Na₂SO₄ and concentrated in vacuo. The residue was purified by preparative TLC (eluent: DCM:MeOH=7:1) to afford (1S,2S)-2-(3-chlorophenyl)-N-(5-(((6-cyclopropylimidazo[1,2-a]pyridin-2-yl)methyl)amino)-1,3,4-thiadiazol-2-yl)cyclopropane-1-carboxamide (45 mg, yield 28%) as white solid. ESI-MS [M+H]+: 465.1, NMR (400 MHz, DMSO) δ 12.19 (s, 1H), 8.33 (s, 1H), 7.79-7.76 (m, 1H), 7.71 (s, 1H), 7.38 (d, J=9.3 Hz, 1H), 7.34-7.25 (m, 3H), 7.15 (d, J=7.6 Hz, 1H), 6.97 (dd, J=9.3, 1.7 Hz, 1H), 4.50 (d, J=5.5 Hz, 2H), 2.47-2.45 (m, 1H), 2.19-2.17 (m, 1H), 1.95-1.88 (m, 1H), 1.53-1.49 (m, 2H), 0.94-0.89 (m, 2H), 0.67-0.65 (m, 2H).

### Example 607

### Synthesis of (1S,2S)-2-(3-chlorophenyl)-N-(4-(((6-cyclopropyl-8-(2-(methylamino)ethyl)imidazo[1,2-a]pyridin-2-yl)methyl)amino)pyridin-2-yl)cyclopropane-1-carboxamide (I-607)

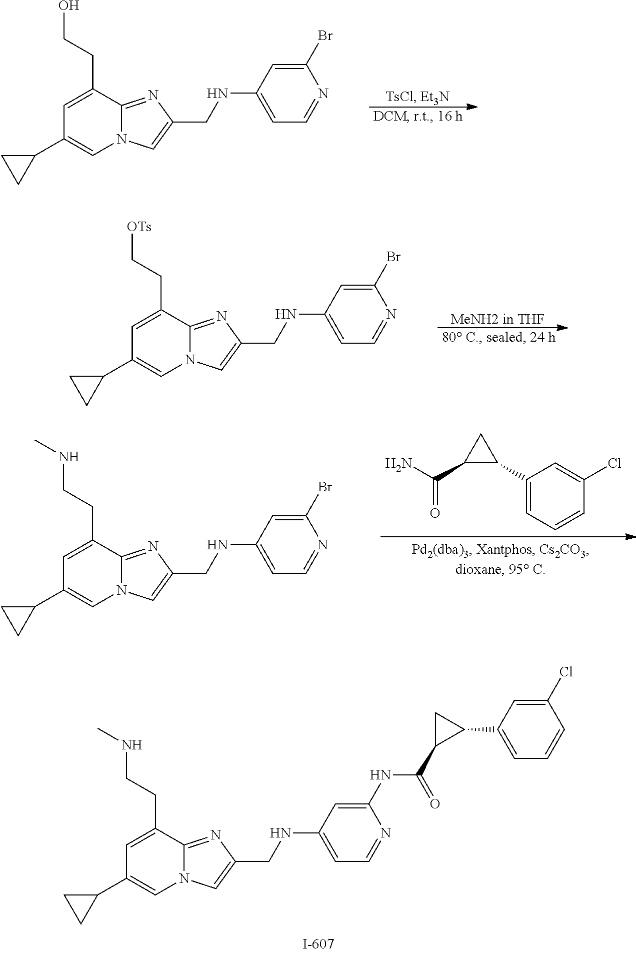 [SMILES: OCCc1cc(C2CC2)cn2cc(CNc3ccnc(Br)c3)nc12 ; Cc1ccc(S(=O)(=O)OCCc2cc(C3CC3)cn3cc(CNc4ccnc(Br)c4)nc23)cc1 ; CNCCc1cc(C2CC2)cn2cc(CNc3ccnc(Br)c3)nc12 ; NC(=O)[C@H]1C[C@@H]1c1cccc(Cl)c1 ; CNCCc1cc(C2CC2)cn2cc(CNc3ccnc(NC(=O)[C@H]4C[C@@H]4c4cccc(Cl)c4)c3)nc12]

Synthesis of 2-(2-(((2-bromopyridin-4-yl)amino)methyl)-6-cyclopropylimidazo[1,2-a]pyridin-8-yl)ethyl 4-methylbenzenesulfonate. To a mixture of 2-(2-(((2-bromopyridin-4-yl)amino)methyl)-6-cyclopropylimidazo-[1,2-a]pyridin-8-yl)ethan-1-ol (38.6 mg, 0.1 mmol) and TEA (30.3 mg, 0.3 mmol) in DCM (5 mL) was added TsCl (28.5 mg, 0.15 mmol) at room temperature. The mixture was stirred at room temperature for 16 h. The reaction was quenched with water (30 mL) and extracted with EtOAc (3×30 mL). The combined organic layers were washed with brine (30 mL), dried over Na₂SO₄ and concentrated in vacuo. The residue was by preparative TLC (eluent: DCM/MeOH=10/1) to give 2-(2-(((2-bromopyridin-4-yl)amino)methyl)-6-cyclopropylimidazo[1,2-a]pyridin-8-yl)ethyl 4-methylbenzenesulfonate (35 mg, 65%) as a yellow solid. ESI-MS [M+H]⁺: 541.2.

Synthesis of 2-bromo-N-((6-cyclopropyl-8-(2-(methylamino)ethyl)imidazo[1,2-a]pyridin-2-yl)methyl)pyridin-4-amine. A mixture of 2-(2-(((2-bromopyridin-4-yl)amino)methyl)-6-cyclopropylimidazo-[1,2-a]pyridin-8-yl)ethyl 4-methylbenzenesulfonate (35 mg, 0.065 mmol) in MeNH₂/THF (2.0 M in THF, 3.0 mL) was stirred at 80° C. in a sealed tube for 24 h. The mixture was concentrated in vacuo and purified by preparative TLC (eluent: DCM/MeOH=10/1) to give 2-bromo-N-((6-cyclopropyl-8-(2-(methylamino)ethyl)imidazo[1,2-a]pyridin-2-yl)methyl)pyridin-4-amine (15.0 mg, 58%) as a light yellow solid. ESI-MS [M+H]⁺: 401.1.

Synthesis of (1S,2S)-2-(3-chlorophenyl)-N-(4-(((6-cyclopropyl-8-(2-(methylamino)ethyl)imidazo-[1,2-a]pyridin-2-yl)methyl)amino)pyridin-2-yl)cyclopropane-1-carboxamide (I-607). A mixture of 2-bromo-N-((6-cyclopropyl-8-(2-(methylamino)ethyl)imidazo[1,2-a]pyridin-2-yl)methyl)pyridin-4-amine (15.0 mg, 0.038 mmol), (1S,2S)-2-(3-chlorophenyl)cyclopropane-1-carboxamide (7.3 mg, 0.038 mmol), Pd₂(dba)₃ (3.4 mg, 0.0038 mmol), Xantphos (2.2 mg, 0.0038 mmol), and Cs₂CO₃ (36.8 mg, 0.11 mmol) in dioxane (2.0 mL) was stirred at 100° C. under N₂ for 5 h. The mixture was quenched with water (30 mL) and extracted with EtOAc (3×30 mL). The combined organic layers were washed with brine (30 mL), dried over Na₂SO₄ and concentrated in vacuo. The residue was purified by preparative TLC (eluent: DCM/MeOH=8/1) to give (1S,2S)-2-(3-chlorophenyl)-N-(4-(((6-cyclopropyl-8-(2-(methylamino)ethyl)imidazo[1,2-a]pyridin-2-yl)methyl)amino)pyridin-2-yl)cyclopropane-1-carboxamide (4.2 mg, 22%) as a white yellow solid. ESI-MS [M+H]⁺: 515.2. ¹H NMR (400 MHz, DMSO) δ 10.31 (s, 1H), 8.16 (s, 1H), 7.75 (d, J=5.7 Hz, 1H), 7.60 (s, 1H), 7.44 (s, 1H), 7.39-7.19 (m, 3H), 7.14 (d, J=7.0 Hz, 2H), 6.81 (s, 1H), 6.32 (d, J=4.0 Hz, 1H), 4.36 (d, J=5.1 Hz, 2H), 3.03-2.94 (m, 2H), 2.91-2.87 (m, 2H), 2.41-2.30 (m, 5H), 1.92-1.80 (m, 1H), 1.48-1.40 (m, 1H), 1.40-1.29 (m, 1H), 0.95-0.83 (m, 2H), 0.70-0.59 (m, 2H).

### Examples 608-609

### Synthesis of (1S,2S)-2-(3-chlorophenyl)-N-(4-(((6-cyclopropyl-8-(3-hydroxy-1-methylazetidin-3-yl)imidazo[1,2-a]pyridin-2-yl)methyl)amino)pyridin-2-yl)cyclopropane-1-carboxamide (I-608) & (1S,2S)-2-(3-chlorophenyl)-N-(4-(((6-cyclopropyl-8-(1-methylazetidin-3-yl)imidazo[1,2-a]pyridin-2-yl)methyl)amino)pyridin-2-yl)cyclopropane-1-carboxamide (I-609)

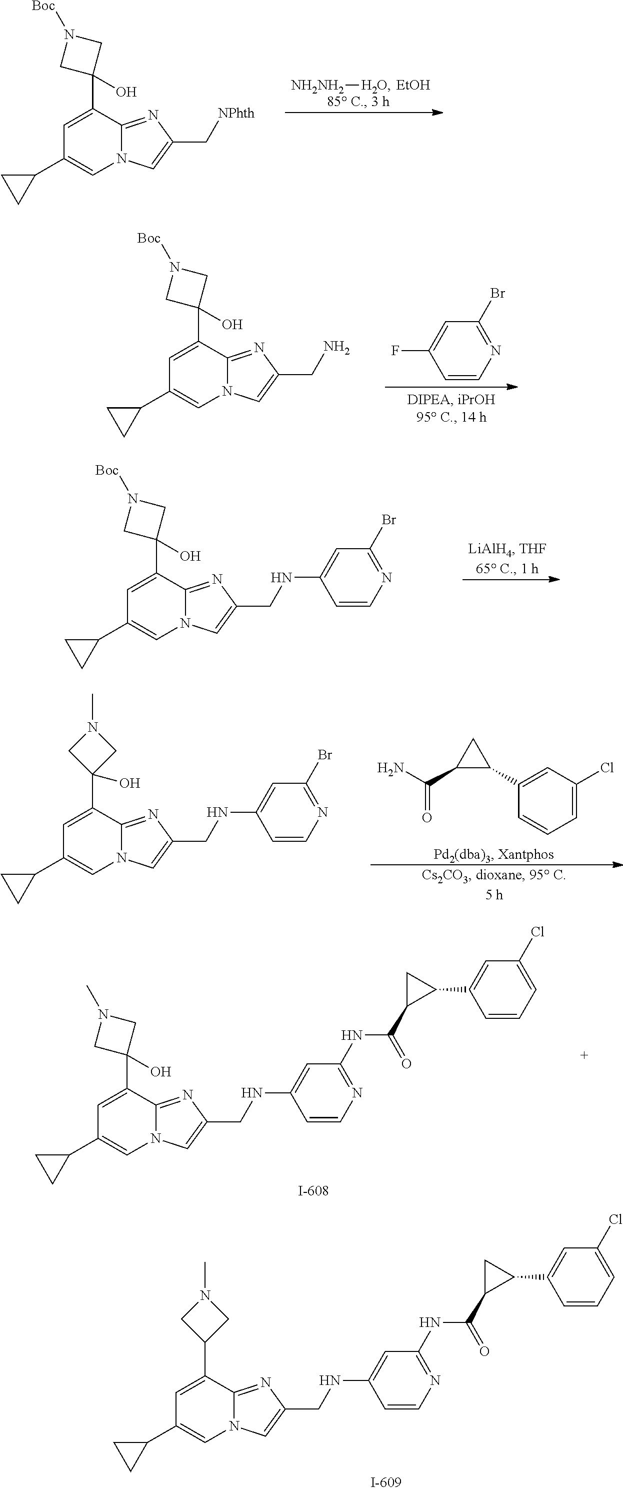

Synthesis of tert-butyl 3-(2-(aminomethyl)-6-cyclopropylimidazo[1,2-a]pyridin-8-yl)-3-hydroxyazetidine-1-carboxylate. To a solution of tert-butyl 3-(6-cyclopropyl-2-((1,3-dioxoisoindolin-2-yl)methyl)imidazo[1,2-a]pyridin-8-yl)-3-hydroxyazetidine-1-carboxylate (1 g, 2.0 mmol)) in EtOH (30 mL) was added hydrazine hydrate (1 g, 20.0 mmol). The mixture was stirred at 85° C. for 3 h and cooled to room temperature. The mixture was filtered and the filtrate was concentrated in vacuo to give tert-butyl 3-(2-(aminomethyl)-6-cyclopropylimidazo[1,2-a]pyridin-8-yl)-3-hydroxyazetidine-1-carboxylate (730 mg, crude) as a yellow solid. ESI-MS [M+H]+: 359.2

Synthesis of tert-butyl 3-(2-(((2-bromopyridin-4-yl)amino)methyl)-6-cyclopropylimidazo[1,2-a]pyridin-8-yl)-3-hydroxyazetidine-1-carboxylate. To a solution of tert-butyl 3-(2-(aminomethyl)-6-cyclopropylimidazo[1,2-a]pyridin-8-yl)-3-hydroxy azetidine-1-carboxylate (700 mg, crude) and 2-bromo-4-fluoropyridine (704 mg, 4.0 mmol) in IPA (30 mL) was added DIPEA (1.29 g, 10.0 mmol). The mixture was stirred at 95° C. for 12 h under N₂. The reaction was quenched with water (50 mL) and extracted with EtOAc (3×50 mL). The combined organic layers were washed with brine (50 mL), dried over Na₂SO₄ and concentrated in vacuo. The residue was purified by column chromatography (eluent: DCM/MeOH=15/1) to give tert-butyl 3-(2-(((2-bromopyridin-4-yl)amino)methyl)-6-cyclopropylimidazo[1,2-a]pyridin-8-yl)-3-hydroxyazetidine-1-carboxylate (530 mg, 52% yield, for two steps) as a pink solid. ESI-MS [M+H]+: 514.1

Synthesis of 3-(2-(((2-bromopyridin-4-yl)amino)methyl)-6-cyclopropylimidazo[1,2-a]pyridin-8-yl)-1-methylazetidin-3-ol. To a solution of tert-butyl 3-(2-(((2-bromopyridin-4-yl)amino)methyl)-6-cyclopropylimidazo[1,2-a]pyridin-8-yl)-3-hydroxyazetidine-1-carboxylate (330 mg, 0.64 mmol) in THF (25 mL) was added LiAlH₄ (3.84 mL, 3.84 mmol, 1M in THF) at 0° C. After stirring at 70° C. for 1 h under N₂, the reaction was quenched with saturated aq·NH₄Cl (50 mL). The aqueous phase was extracted with EtOAc (3×30 mL). The combined organic layers were washed with brine (30 mL), dried over Na₂SO₄, then concentrated in vacuo to give the crude, which was purified by Pre-TLC (eluent: DCM/MeOH=10/1) to give the product 3-(2-(((2-bromopyridin-4-yl)amino)methyl)-6-cyclopropylimidazo[1,2-a]pyridin-8-yl)-1-methylazetidin-3-ol (130 mg, 47%) as a yellow solid. ESI-MS [M+H]+: 428.1

Synthesis of (1S,2S)-2-(3-chlorophenyl)-N-(4-(((6-cyclopropyl-8-(3-hydroxy-1-methylazetidin-3-yl)imidazo[1,2-a]pyridin-2-yl)methyl)amino)pyridin-2-yl)cyclopropane-1-carboxamide (I-608) and (1S,2S)-2-(3-chlorophenyl)-N-(4-(((6-cyclopropyl-8-(1-methylazetidin-3-yl)imidazo[1,2-a]pyridin-2-yl)methyl)amino)pyridin-2-yl)cyclopropane-1-carboxamide (I-609). A mixture of 3-(2-(((2-bromopyridin-4-yl)amino)methyl)-6-cyclopropylimidazo[1,2-a]pyridin-8-yl)-1-methylazetidin-3-ol (130 mg, 0.30 mmol), (1S,2S)-2-(3-chlorophenyl)cyclopropane-1-carboxamide (66 mg, 0.33 mmol), Pd₂(dba)₃ (56 mg, 0.068 mmol), Xantphos (35 mg, 0.068 mmol), and Cs₂CO₃ (298 mg, 0.91 mmol) in dioxane (15 mL) was stirred at 95° C. for 5 h. The reaction was quenched with water (50 mL) and extracted with EtOAc (3×30 mL). The combined organic layers were washed with brine (30 mL), dried over Na₂SO₄, and concentrated in vacuo to give the crude, which was purified by Pre-TLC (eluent: DCM/MeOH=10/1) to give the product (1S,2S)-2-(3-chlorophenyl)-N-(4-(((6-cyclopropyl-8-(3-hydroxy-1-methylazetidin-3-yl)imidazo[1,2-a]pyridin-2-yl)methyl)amino)pyridin-2-yl)cyclopropane-1-carboxamide (10 mg, 12%) as a white solid. ESI-MS [M+H]+: 543.2. ¹H NMR (400 MHz, DMSO) δ 10.32 (s, 1H), 8.25 (d, J=1.2 Hz, 1H), 7.80-7.72 (m, 1H), 7.65 (s, 1H), 7.45 (s, 1H), 7.31 (t, J=7.9 Hz, 1H), 7.26-7.23 (m, 2H), 7.15-7.10 (m, 3H), 6.43 (s, 1H), 6.34-6.32 (m, 1H), 4.43-4.35 (m, 2H), 3.97 (d, J=6.3 Hz, 2H), 3.37-3.35 (m, 2H), 2.42 (s, 3H), 2.34-2.32 (m, 2H), 2.03-1.91 (m, 1H), 1.49-1.40 (m, 1H), 1.38-1.31 (m, 1H), 0.94-0.89 (m, 2H), 0.70-0.65 (m, 2H).

Also isolated was (1S,2S)-2-(3-chlorophenyl)-N-(4-(((6-cyclopropyl-8-(1-methylazetidin-3-yl)imidazo[1,2-a]pyridin-2-yl)methyl)amino)pyridin-2-yl)cyclopropane-1-carboxamide (10 mg, 12%) as a white solid. ESI-MS [M+H]+: 527.2, ¹H NMR (400 MHz, DMSO) δ 10.31 (s, 1H), 8.17 (s, 1H), 7.75 (d, J=5.8 Hz, 1H), 7.60 (s, 1H), 7.43 (s, 1H), 7.31 (t, J=7.9 Hz, 1H), 7.26-7.23 (m, 2H), 7.15-7.09 (m, 2H), 6.94 (s, 1H), 6.33-6.31 (m, 1H), 4.35 (d, J=5.8 Hz, 2H), 4.04-3.96 (m, 1H), 3.71 (t, J=7.4 Hz, 2H), 3.29-3.22 (m, 2H), 2.40-2.33 (m, 2H), 2.30 (s, 3H), 1.93-1.89 (m, 1H), 1.46-1.42 (m, 1H), 1.37-1.34 (m, 1H), 0.93-0.87 (m, 2H), 0.71-0.66 (m, 2H).

### Example 610

### Synthesis of (1S,2S)-2-(3-chlorophenyl)-N-(5-(((6-cyclopropylimidazo[1,2-a]pyridin-2-yl)methyl)amino)-1,2,4-thiadiazol-3-yl)cyclopropane-1-carboxamide (I-610)

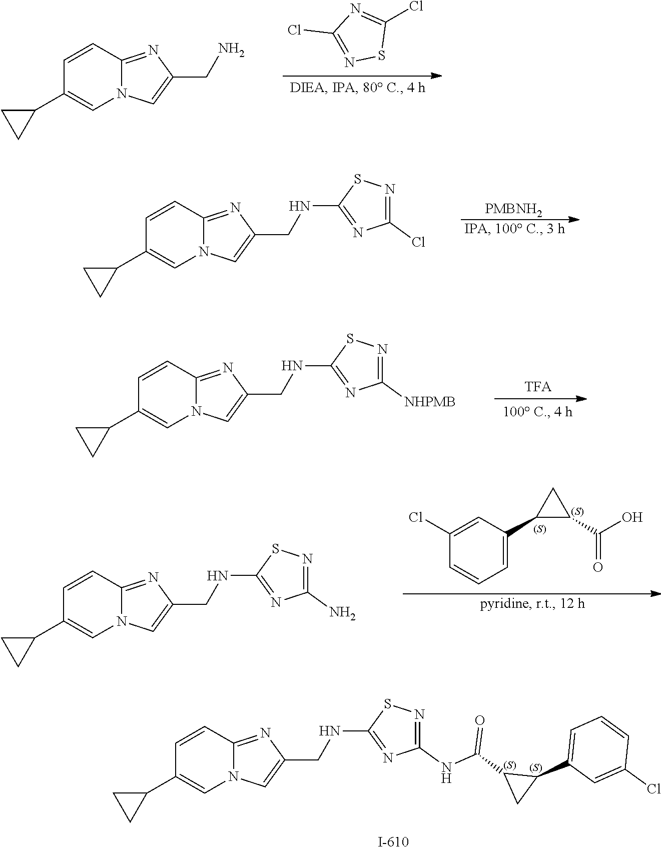

Synthesis of 3-chloro-N-((6-cyclopropylimidazo[1,2-a]pyridin-2-yl)methyl)-1,2,4-thiadiazol-5-amine. A solution of (6-cyclopropylimidazo[1,2-a]pyridin-2-yl)methanamine (0.5 g, 2.67 mmol), 3,5-dichloro-1,2,4-thiadiazole (494 mg, 3.21 mmol), and DIPEA (1.03 g, 8.01 mmol) in IPA (20 mL) was stirred at 80° C. for 4 h. The reaction was quenched with water (30 mL) and extracted with EtOAc (3×30 mL). The combined organic layers were washed with brine (30 mL), dried over Na₂SO₄ and concentrated in vacuo to give the crude, which was purified by silica gel column chromatography (eluent: DCM/MeOH=10/1) to give the 3-chloro-N-((6-cyclopropylimidazo[1,2-a]pyridin-2-yl)methyl)-1,2,4-thiadiazol-5-amine as a yellow solid. (400 mg, 49%), ESI-MS [M+H]+: 306.2

Synthesis of N5-((6-cyclopropylimidazo[1,2-a]pyridin-2-yl)methyl)-N3-(4-methoxybenzyl)-1,2,4-thiadiazole-3,5-diamine. A solution of 3-chloro-N-((6-cyclopropylimidazo[1,2-a]pyridin-2-yl)methyl)-1,2,4-thiadiazol-5-amine (500 mg, 1.64 mmol) and (4-methoxyphenyl)methanamine (1.12 g, 8.20 mmol) in DMSO (10 mL) was stirred at 100° C. for 3 h. The reaction was quenched with water (100 mL) and extracted with EtOAc (3×30 mL). The combined organic layers were washed with brine (30 mL), dried over Na₂SO₄ then concentrated in vacuo to give N5-((6-cyclo propylimidazo[1,2-a]pyridin-2-yl)methyl)-N3-(4-methoxybenzyl)-1,2,4-thiadiazole-3,5-diamine as a white solid (350 mg, crude) which was used without further purification. ESI-MS [M+H]+: 407.2.

Synthesis of N5-((6-cyclopropylimidazo[1,2-a]pyridin-2-yl)methyl)-1,2,4-thiadiazole-3,5-diamine. A solution of N5-((6-cyclopropylimidazo[1,2-a]pyridin-2-yl)methyl)-N3-(4-methoxybenzyl)-1,2,4-thiadiazole-3,5-diamine (400 mg, 0.99 mmol) in TFA (5 mL) was stirred at 100° C. for 4 h. The reaction was quenched with saturated aq·NaHCO₃ (100 mL) and extracted with EtOAc (3×30 mL). The combined organic layers were washed with brine (30 mL), dried over Na₂SO₄, concentrated in vacuo and purified by silica gel column chromatography (eluent: DCM/MeOH=10/1) to give N5-((6-cyclopropylimidazo[1,2-a]pyridin-2-yl)methyl)-1,2,4-thiadiazole-3,5-diamine (200 mg, 71% yield) as a white solid. ESI-MS [M+H]+: 287.2.

Synthesis of (1S,2S)-2-(3-chlorophenyl)-N-(5-(((6-cyclopropylimidazo[1,2-a]pyridin-2-yl)methyl)amino)-1,2,4-thiadiazol-3-yl)cyclopropane-1-carboxamide (I-610). To a solution of (1S,2S)-2-(3-chlorophenyl)cyclopropane-1-carboxylic acid (33 mg, 0.17 mmol) in DCM (5 ml) was added oxalyl dichloride (43 mg, 0.34 mmol) and DMF (3 drops) at 0° C. under N2. The mixture was stirred at 0° C. for 3 h and evaporated to give the product as a light-yellow oil (36 mg, crude). DCM (3 mL) was added, followed by N5-((6-cyclopropylimidazo[1,2-a]pyridin-2-yl)methyl)-1,2,4-thiadiazole-3,5-diamine (50 mg, 0.17 mmol) and pyridine (1 mL). The mixture was stirred at room temperature for 12 h, then quenched with saturated aq·NaHCO₃ (50 mL) and extracted with EtOAc (3×10 mL). The combined organic layers were washed with brine (30 mL), dried over Na₂SO₄ and concentrated in vacuo to give the crude, which was purified by silica gel column chromatography (eluent: DCM/MeOH=10/1) to give (1S,2S)-2-(3-chlorophenyl)-N-(5-(((6-cyclopropylimidazo[1,2-a]pyridin-2-yl)methyl)amino)-1,2,4-thiadiazol-3-yl)cyclopropane-1-carboxamide as a white solid. (20 mg, 25%). ESI-MS [M+H]⁺: 465.0, ¹H NMR (400 MHz, DMSO) δ 10.89 (s, 1H), 8.87 (s, 1H), 8.32 (s, 1H), 7.72 (s, 1H), 7.40 (d, J=9.3 Hz, 1H), 7.35-7.27 (m, 1H), 7.27-7.21 (m, 2H), 7.13 (d, J=7.6 Hz, 1H), 7.01-6.98 (m, 1H), 4.54 (d, J=4.7 Hz, 2H), 2.38-2.32 (m, 2H), 1.97-1.88 (m, 1H), 1.48-1.43 (m, 1H), 1.37-1.33 (m, 1H), 0.95-0.89 (m, 2H), 0.70-0.62 (m, 2H).

### Example 611

### Synthesis of (1S,2S)-2-(3-chlorophenyl)-N-(4-(((6-cyclopropylimidazo[1,2-a]pyridin-2-yl)methyl)amino)-6-hydroxypyridin-2-yl)cyclopropane-1-carboxamide (I-611)

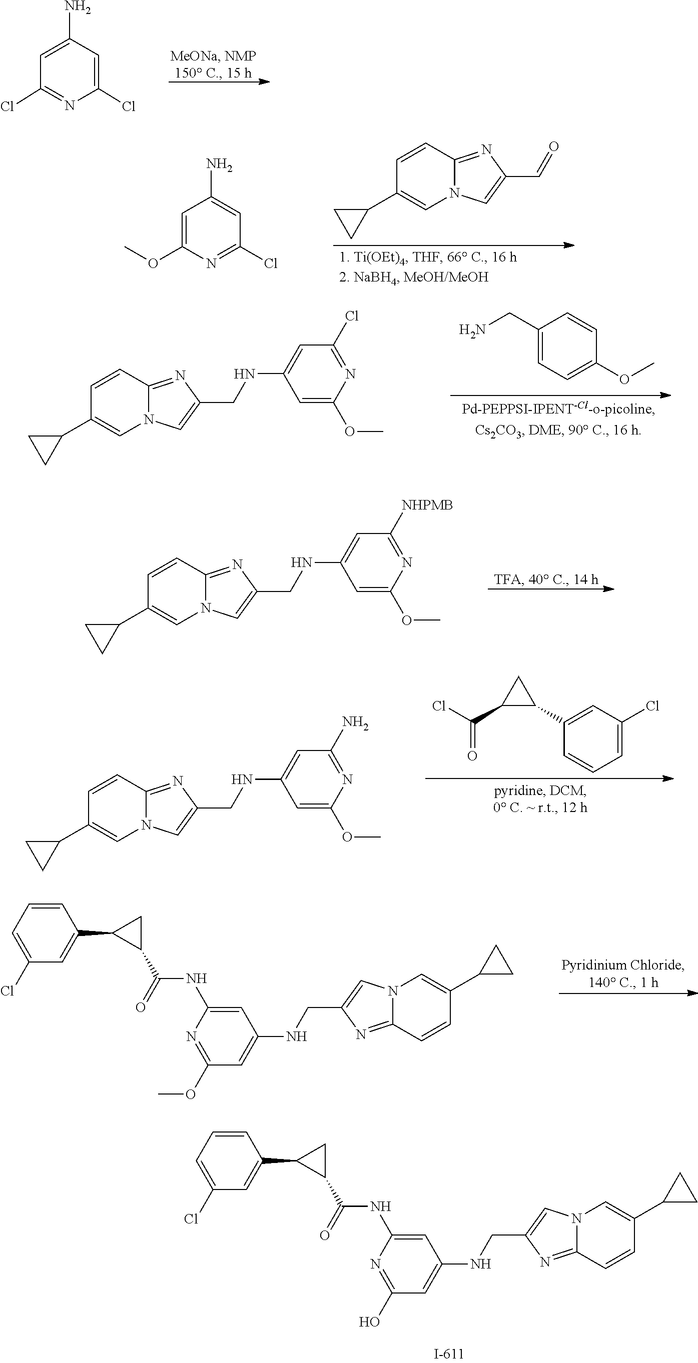

Synthesis of 2-chloro-6-methoxypyridin-4-amine. A mixture of 2,6-dichloropyridin-4-amine (1 g, 6.13 mmol) and NaOMe (1 g, 19.39 mmol) in NMP (40 mL) was stirred at 150° C. for 15 h. After cooling to room temperature. the reaction was quenched with water (80 mL) and extracted with EtOAc (3×80 mL). The combined organic layers were washed with brine (50 mL), dried over Na₂SO₄, and concentrated in vacuo to give the crude, which was purified by silica gel column chromatography (eluent: PE/EtAOc=3/1) to give 2-chloro-6-methoxypyridin-4-amine (400 mg, 40.8%) as yellow oil.

ESI-MS [M+H]+: 159.2

Synthesis of 2-chloro-N-((6-cyclopropylimidazo[1,2-a]pyridin-2-yl)methyl)-6-methoxypyridin-4-amine. A mixture of 2-chloro-6-methoxypyridin-4-amine (400 mg, 2.51 mmol), 6-cyclopropylimidazo[1,2-a]pyridine-2-carbaldehyde (466.9 mg, 2.51 mmol) and Ti(OEt)₄ (1.14 g, 5.02 mmol) in THF (20 mL) was stirred at 66° C. for 16 h. The reaction was cooled to room temperature, and then MeOH (10 mL) was added, followed by NaBH₄ (286 mg, 7.53 mmol). The resulting reaction mixture was stirred at room temperature for another 2 h. The reaction was quenched with saturated aq·NH₄Cl (30 mL) and extracted with DCM (3×50 mL). The combined organic layers were washed with brine (30 mL), dried over Na₂SO₄, and concentrated in vacuo to give the crude, which was purified with silica gel column chromatography (eluent: DCM/MeOH=15/1) to give 2-chloro-N-((6-cyclopropylimidazo[1,2-a]pyridin-2-yl)methyl)-6-methoxypyridin-4-amine (350 mg, 43%) as a yellow solid. ESI-MS [M+H]+: 329.2

Synthesis of N4-((6-cyclopropylimidazo[1,2-a]pyridin-2-yl)methyl)-6-methoxy-N2-(4-methoxybenzyl)pyridine-2,4-diamine. A mixture of 2-chloro-N-((6-cyclopropylimidazo[1,2-a]pyridin-2-yl)methyl)-6-methoxypyridin-4-amine (350 mg, 1.1 mmol), (4-methoxyphenyl)methanamine (370 mg, 2.7 mmol), Pd-PEPPSI-IPENT⁻Cl-o-picoline (92.4 mg, 0.11 mmol), and Cs₂CO₃ (1.1 g, 3.3 mmol) in DME (25 mL) was stirred at 90° C. for 16 h. The reaction was cooled to room temperature and filtered. The filtrate was concentrated in vacuo to give the crude, which was purified with preparative TLC (eluent: DCM/MeOH=15/1) to give N4-((6-cyclopropylimidazo[1,2-a]pyridin-2-yl)methyl)-6-methoxy-N2-(4-methoxybenzyl)pyridine-2,4-diamine (265 mg, 56%) as a yellow solid. ESI-MS [M+H]+: 430.2

Synthesis of N4-((6-cyclopropylimidazo[1,2-a]pyridin-2-yl)methyl)-6-methoxypyridine-2,4-diamine. A solution of N4-((6-cyclopropylimidazo[1,2-a]pyridin-2-yl)methyl)-6-methoxy-N2-(4-methoxybenzyl)pyridine-2,4-diamine (265 mg, 0.62 mmol) in TFA (10 mL) was stirred at 40° C. for 14 h. The reaction was concentrated in vacuo to give the residue, which was washed with saturated aq·NaHCO₃ (30 mL) and extracted with EtOAc (3×50 mL). The combined organic layers were washed with brine (40 mL), dried over Na₂SO₄, and concentrated in vacuo to give the crude, which was purified with preparative TLC (eluent: DCM/MeOH=10/1) to give N4-((6-cyclopropylimidazo[1,2-a]pyridin-2-yl)methyl)-6-methoxypyridine-2,4-diamine (120 mg, 63%) as a yellow solid. ESI-MS [M+H]+: 310.2

Synthesis of (1S,2S)-2-(3-chlorophenyl)-N-(4-(((6-cyclopropylimidazo[1,2-a]pyridin-2-yl)methyl)amino)-6-methoxypyridin-2-yl)cyclopropane-1-carboxamide. To a solution of N4-((6-cyclopropylimidazo[1,2-a]pyridin-2-yl)methyl)-6-methoxypyridine-2,4-diamine (120 mg, 0.39 mmol) in pyridine/DCM (2 mL/10 mL) was added (1S,2S)-2-(3-chlorophenyl)cyclopropane-1-carbonyl chloride (167.7 mg, 0.78 mmol) at 0° C. The resulting reaction mixture was stirred at room temperature for 12 h. The reaction was quenched with saturated aq·NH₄Cl (20 mL) and extracted with DCM (3×30 mL). The combined organic layers were washed with brine (30 mL), dried over Na₂SO₄, and concentrated in vacuo to give crude, which was purified with preparative TLC (eluent: DCM/MeOH=10/1) to give (1S,2S)-2-(3-chlorophenyl)-N-(4-(((6-cyclopropylimidazo[1,2-a]pyridin-2-yl)methyl)amino)-6-methoxypyridin-2-yl)cyclopropane-1-carboxamide (75 mg, 39%) as a yellow solid. ESI-MS [M+H]+: 488.2

Synthesis of (1S,2S)-2-(3-chlorophenyl)-N-(4-(((6-cyclopropylimidazo[1,2-a]pyridin-2-yl)methyl)amino)-6-hydroxypyridin-2-yl)cyclopropane-1-carboxamide (I-611). A mixture of (1S,2S)-2-(3-chlorophenyl)-N-(4-(((6-cyclopropylimidazo[1,2-a]pyridin-2-yl)methyl)amino)-6-methoxypyridin-2-yl)cyclopropane-1-carboxamide (75 mg, 0.15 mmol) and pyridinium chloride (345 mg, 3 mmol) was stirred at 140° C. for 1 h. The reaction was washed with water (30 mL) and extracted with EtOAc (3×40 mL). The combined organic layers were washed with brine (40 mL), dried over Na₂SO₄, and concentrated in vacuo to give the crude, which was purified with silica gel column chromatography (eluent: DCM/MeOH=10/1) to give (1S,2S)-2-(3-chlorophenyl)-N-(4-(((6-cyclopropylimidazo[1,2-a]pyridin-2-yl)methyl)amino)-6-hydroxypyridin-2-yl)cyclopropane-1-carboxamide (15 mg, 21%) as a white solid. ESI-MS [M+H]+: 474.2, ¹H NMR (400 MHz, DMSO) δ 10.67 (s, 1H), 10.45 (s, 1H), 8.32 (s, 1H), 7.65 (s, 1H), 7.38 (d, J=9.3 Hz, 1H), 7.33-7.25 (m, 3H), 7.17 (d, J=7.6 Hz, 1H), 7.10 (t, J=3.8 Hz, 1H), 6.98-6.96 (m, 1H), 5.69 (s, 1H), 5.05 (s, 1H), 4.27 (d, J=8.0 Hz, 2H), 2.47-2.42 (m, 2H), 1.94-1.88 (m, 1H), 1.54-1.50 (m, 1H), 1.48-1.43 (m, 1H), 0.93-0.87 (m, 2H), 0.71-0.65 (m, 2H).

### Example 612

### Synthesis of (1S,2S)-2-(3-chlorophenyl)-N-(5-(((6-cyclopropyl-8-(4-methylpiperazin-1-yl)imidazo[1,2-a]pyridin-2-yl)methyl)amino)pyridazin-3-yl)cyclopropane-1-carboxamide (I-612)

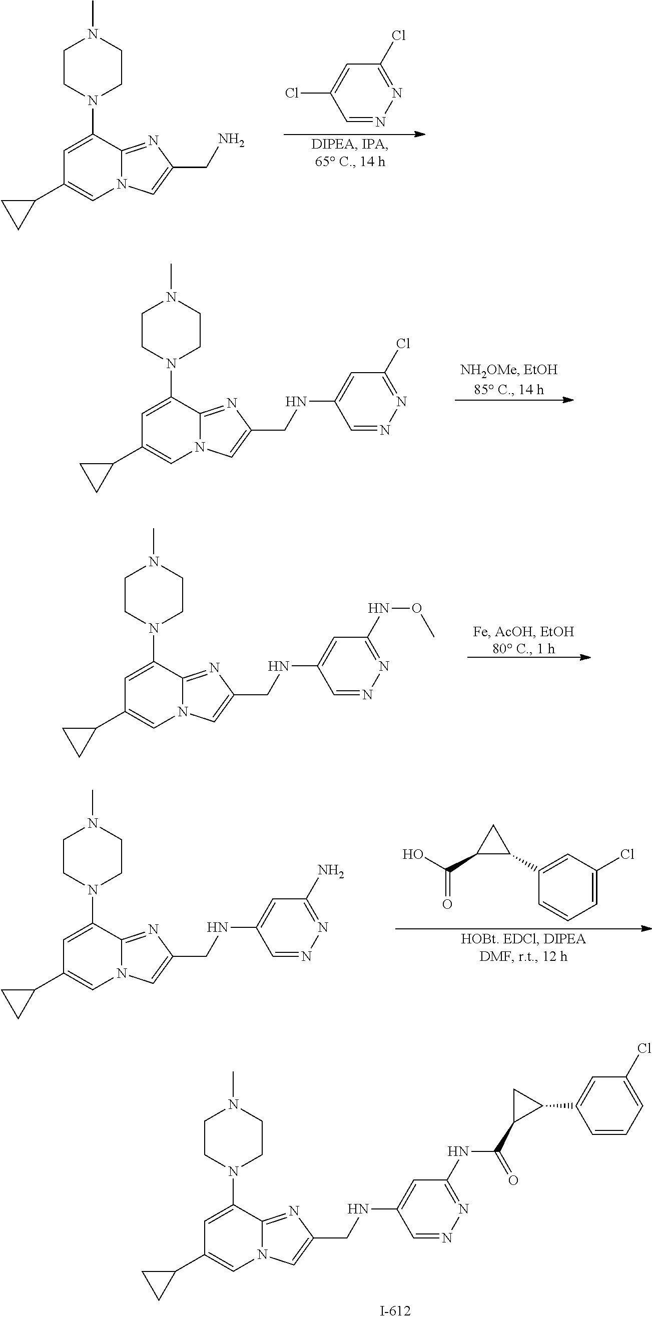

Synthesis of 6-chloro-N-((6-cyclopropyl-8-(4-methylpiperazin-1-yl)imidazo[1,2-a]pyridin-2-yl)methyl)pyridazin-4-amine. A mixture of (6-cyclopropyl-8-(4-methylpiperazin-1-yl)imidazo[1,2-a]pyridin-2-yl)methanamine (570 mg, 2 mmol), 3,5-dichloropyridazine (298 mg, 2 mmol), and DIPEA (1.42 mL, 18 mmol) in IPA (15 mL) was stirred at 65° C. for 14 h. The mixture was quenched with saturated aq·NaHCO₃ (50 mL) and extracted with EtOAc (3×50 mL). The combined organic layers were washed with brine (30 mL), dried over Na₂SO₄ and concentrated in vacuo. The residue was purified by column chromatography (eluent: DCM/MeOH=15/1) to give 6-chloro-N-((6-cyclopropyl-8-(4-methylpiperazin-1-yl)imidazo[1,2-a]pyridin-2-yl)methyl)pyridazin-4-amine (350 mg, 46%) as a yellow solid. ESI-MS [M+H]+: 398.2

Synthesis of N-((6-cyclopropyl-8-(4-methylpiperazin-1-yl)imidazo[1,2-a]pyridin-2-yl)methyl)-6-(methoxyamino)pyridazin-4-amine. A mixture of 6-chloro-N-((6-cyclopropyl-8-(4-methylpiperazin-1-yl)imidazo[1,2-a]pyridin-2-yl)methyl)pyridazin-4-amine (200 mg, 0.5 mmol) and NH₂OMe (236 mg, 5 mmol) in EtOH (10 mL) was stirred at 85° C. for 14 h. The reaction was quenched with saturated aq·NaHCO₃ (50 mL) and extracted with EtOAc (3×50 mL). The combined organic layers were washed with brine (30 mL), dried over Na₂SO₄ and concentrated in vacuo. The residue was purified by preparative TLC (eluent: DCM/MeOH=15/1) to give N-((6-cyclopropyl-8-(4-methylpiperazin-1-yl)imidazo[1,2-a]pyridin-2-yl)methyl)-6-(methoxyamino)pyridazin-4-amine (160 mg, 78%) as a yellow solid. ESI-MS [M+H]+: 409.2

Synthesis of N5-((6-cyclopropyl-8-(4-methylpiperazin-1-yl)imidazo[1,2-a]pyridin-2-yl)methyl)pyridazine-3,5-diamine. A mixture of N-((6-cyclopropyl-8-(4-methylpiperazin-1-yl)imidazo[1,2-a]pyridin-2-yl)methyl)-6-(methoxyamino)pyridazin-4-amine (160 mg, 0.39 mmol) and Fe (109 mg, 1.95 mmol) in AcOH/EtOH (2 mL/10 mL) was stirred at 80° C. for 1 h. The reaction mixture was filtered and washed with MeOH (20 mL). The combined filtrate was concentrated in vacuo to give N5-((6-cyclopropyl-8-(4-methylpiperazin-1-yl)imidazo[1,2-a]pyridin-2-yl)methyl)pyridazine-3,5-diamine (100 mg, 68%) as a yellow solid which was used in next step directly without further purification. ESI-MS [M+H]+: 379.2

Synthesis of (1S,2S)-2-(3-chlorophenyl)-N-(5-(((6-cyclopropyl-8-(4-methylpiperazin-1-yl)imidazo[1,2-a]pyridin-2-yl)methyl)amino)pyridazin-3-yl)cyclopropane-1-carboxamide (I-612). A mixture of N5-((6-cyclopropyl-8-(4-methylpiperazin-1-yl)imidazo[1,2-a]pyridin-2-yl)methyl)pyridazine-3,5-diamine (60 mg, 0.16 mmol), (1S,2S)-2-(3-chlorophenyl)cyclopropane-1-carboxylic acid (37 mg, 0.19 mmol), HOBt (33 mg, 0.24 mmol), EDCI (46 mg, 0.24 mmol), and DIPEA (83 mg, 0.64 mmol) in DMF (3 mL) was stirred at room temperature for 12 h. The reaction was quenched with water (30 mL) and extracted with EtOAc (4×25 mL). The combined organic layers were washed with brine (30 mL), dried over Na₂SO₄, and concentrated in vacuo to give the crude, which was purified with preparative TLC (eluent: DCM/MeOH=15/1) to give (1S,2S)-2-(3-chlorophenyl)-N-(5-(((6-cyclopropyl-8-(4-methylpiperazin-1-yl)imidazo[1,2-a]pyridin-2-yl)methyl)amino)pyridazin-3-yl)cyclopropane-1-carboxamide (10 mg, 11%) as a white solid. ESI-MS [M+H]+: 557.2, ¹H NMR (400 MHz, DMSO) δ 10.84 (s, 1H), 8.40 (d, J=1.4 Hz, 1H), 7.87 (s, 1H), 7.66-7.53 (m, 3H), 7.33-7.25 (m, 3H), 7.15 (d, J=7.5 Hz, 1H), 6.16 (s, 1H), 4.37 (d, J=5.4 Hz, 2H), 3.55-3.45 (m, 4H), 2.61-2.53 (m, 4H), 2.41-2.38 (m, 2H), 2.26 (s, 3H), 1.88-1.82 (m, 1H), 1.47-1.44 (m, 1H), 1.42-1.39 (m, 1H), 0.89-0.84 (m, 2H), 0.69-0.63 (m, 2H).

### Example 613

### Synthesis of (1S,2S)-2-(3-chlorophenyl)-N-(2-(((6-cyclopropyl-8-(4-methylpiperazin-1-yl)imidazo[1,2-a]pyridin-2-yl)methyl)amino)pyrimidin-4-yl)cyclopropane-1-carboxamide (I-613)

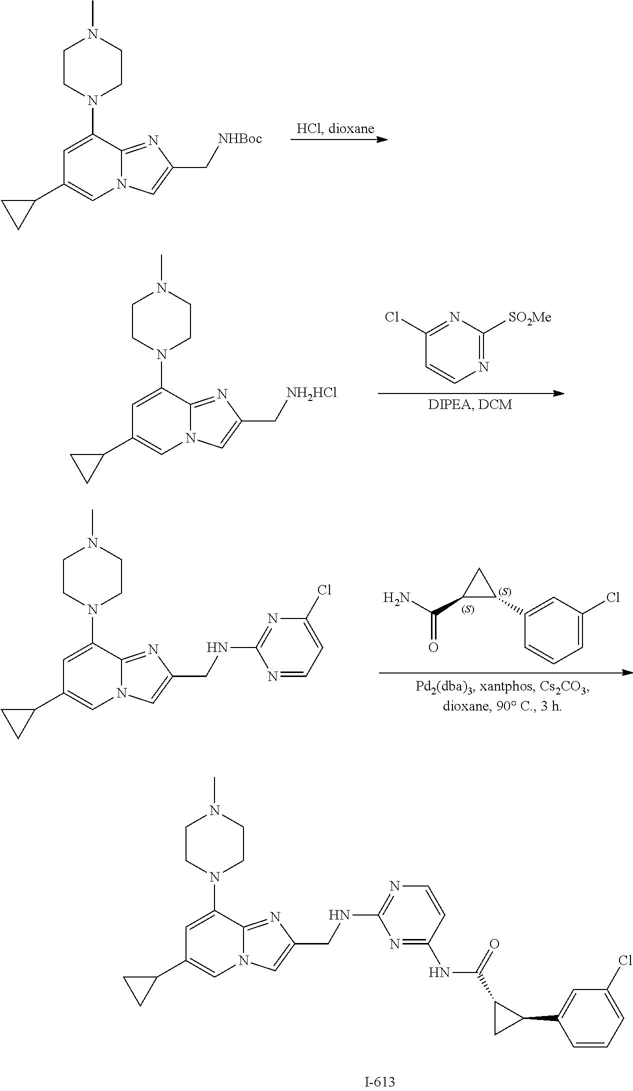

Synthesis of (6-cyclopropyl-8-(4-methylpiperazin-1-yl)imidazo[1,2-a]pyridin-2-yl)methanamine hydrochloride. HCl (4M solution in dioxane, 5 mL) was added to a solution of tert-butyl ((6-cyclopropyl-8-(4-methylpiperazin-1-yl)imidazo[1,2-a]pyridin-2-yl)-methyl)carbamate (385 mg, 1.0 mmol) in MeOH (5 mL). The mixture was stirred at room temperature for 1 h and concentrated in vacuo to give (6-cyclopropyl-8-(4-methylpiperazin-1-yl)imidazo[1,2-a]pyridin-2-yl)methanamine as the hydrochloric acid salt (300 mg, 94%) as a yellow solid which was used in next step directly without further purification. ESI-MS [M+H]+: 286.1.

Synthesis of 4-chloro-N-((6-cyclopropyl-8-(4-methylpiperazin-1-yl)imidazo[1,2-a]pyridin-2-yl-)methyl)pyrimidin-2-amine. A mixture of (6-cyclopropyl-8-(4-methylpiperazin-1-yl)imidazo[1,2-a]pyridin-2-yl)methanamine hydrochloride (200 mg, 0.63 mmol), 4-chloro-2-(methylsulfonyl)pyrimidine (121.0 mg, 0.63 mmol), and DIPEA (162.5 mg, 1.26 mmol) in dry DCM (20 mL) was stirred at room temperature for 1 h. The mixture was quenched with saturated aq·NaHCO₃ (50 mL) and extracted with EtOAc (3×50 mL). The combined organic layers were washed with brine (30 mL), dried over Na₂SO₄ and concentrated in vacuo. The residue was purified by preparative TLC (eluent: DCM/MeOH=10/1) to give 4-chloro-N-((6-cyclopropyl-8-(4-methylpiperazin-1-yl)imidazo[1,2-a]pyridin-2-yl)methyl)pyrimidin-2-amine (30 mg, 12%) as a yellow solid. ESI-MS [M+H]+: 398.2.

Synthesis of (1S,2S)-2-(3-chlorophenyl)-N-(2-(((6-cyclopropyl-8-(4-methylpiperazin-1-yl)imidazo[1,2-a]pyridin-2-yl)methyl)amino)pyrimidin-4-yl)cyclopropane-1-carboxamide (I-613). To a mixture of 4-chloro-N-((6-cyclopropyl-8-(4-methylpiperazin-1-yl)imidazo[1,2-a]-pyridin-2-yl)methyl)pyrimidin-2-amine (30 mg, 0.076 mmol), (1S,2S)-2-(3-chlorophenyl)cyclopropane-1-carboxamide (14.7 mg, 0.076 mmol), and Cs₂CO₃ (74.3 mg, 0.228 mmol) in 1,4-dioxane (5 mL) were added Pd₂(dba)₃ (7.0 mg, 0.0076 mmol) and Xantphos (8.8 mg, 0.015 mmol). After stirring at 90° C. for 3 h under N₂, the reaction mixture was filtered through Celite® and the filter cake was washed with DCM/MeOH (10/1, 20 mL). The filtrate was concentrated in vacuo to give the crude, which was purified by preparative HPLC (eluent: DCM/MeOH=10/1) to give (1S,2S)-2-(3-chlorophenyl)-N-(2-(((6-cyclopropyl-8-(4-methylpiperazin-1-yl)imidazo[1,2-a]pyridin-2-yl)methyl)amino)pyrimidin-4-yl)cyclopropane-1-carboxamide (13.1 mg, 31%) as a white solid. ESI-MS [M+H]⁺: 557.2, ¹H NMR (400 MHz, MeOD) δ 8.15 (d, J=5.9 Hz, 1H), 7.95 (s, 1H), 7.71 (s, 1H), 7.43 (d, J=5.9 Hz, 1H), 7.26-7.20 (m, 1H), 7.23-7.14 (m, 2H), 7.11 (d, J=7.6 Hz, 1H), 6.69 (s, 1H), 4.72 (s, 2H), 3.71-3.39 (m, 4H), 3.31-3.30 (m, 4H), 3.00 (s, 3H), 2.57-2.45 (m, 1H), 2.27-2.13 (m, 1H), 1.99-1.91 (m, 1H), 1.70-1.59 (m, 1H), 1.47-1.35 (m, 1H), 1.05-0.91 (m, 2H), 0.80-0.65 (m, 2H).

### Example 614

### Synthesis of rac-(1S*,2S*)-2-(3-chloropyridinyl)-N-(5-(((6-cyclopropylimidazo[1,2-a]pyridin-2-yl)methyl)amino)-1,2,4-thiadiazol-3-yl)cyclopropane-1-carboxamide (I-614)

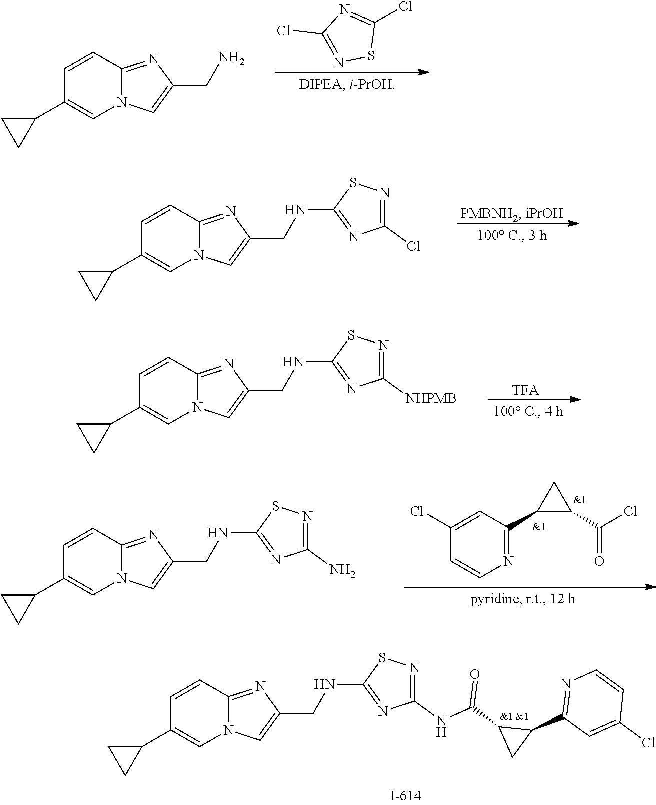

Synthesis of 3-chloro-N-((6-cyclopropylimidazo[1,2-a]pyridin-2-yl)methyl)-1,2,4-thiadiazol-5-amine. A solution of (6-cyclopropylimidazo[1,2-a]pyridin-2-yl)methanamine (0.5 g, 2.67 mmol), 3,5-dichloro-1,2,4-thiadiazole (494 mg, 3.21 mmol) and DIPEA (1.03 g, 8.01 mmol) in i-PrOH (20 mL) was stirred at 80° C. for 4 h. The reaction was quenched with water (30 mL) and extracted with EtOAc (3×30 mL). The combined organic layers were washed with brine, dried over Na₂SO₄, and concentrated in vacuo to give the crude, which was purified by silica gel column chromatography (eluent: DCM/MeOH=10/1) to give 3-chloro-N-((6-cyclopropylimidazo[1,2-a]pyridin-2-yl)methyl)-1,2,4-thiadiazol-5-amine (400 mg, 49%) as a yellow solid. ESI-MS [M+H]+: 306.2

Synthesis of N5-((6-cyclopropylimidazo[1,2-a]pyridin-2-yl)methyl)-N3-(4-methoxybenzyl)-1,2,4-thiadiazole-3,5-diamine. A solution of 3-chloro-N-((6-cyclopropylimidazo[1,2-a]pyridin-2-yl)methyl)-1,2,4-thiadiazol-5-amine (500 mg, 1.64 mmol) and (4-methoxyphenyl)methanamine (1.12 g, 8.20 mmol) in DMSO (10 mL) was stirred at 100° C. for 3 h. The reaction was quenched with water (25 mL) and extracted with EtOAc (3×30 mL). The combined organic layers were washed with brine, dried over Na₂SO₄, and concentrated in vacuo to give N5-((6-cyclopropylimidazo[1,2-a]pyridin-2-yl)methyl)-N3-(4-methoxybenzyl)-1,2,4-thiadiazole-3,5-diamine (350 mg, crude) as a white solid. ESI-MS [M+H]+: 407.2.

Synthesis of N5-((6-cyclopropylimidazo[1,2-a]pyridin-2-yl)methyl)-1,2,4-thiadiazole-3,5-diamine. A solution of N5-((6-cyclopropylimidazo[1,2-a]pyridin-2-yl)methyl)-N3-(4-methoxybenzyl)-1,2,4-thiadiazole-3,5-diamine (400 mg, 0.99 mmol) in TFA (5 mL) was stirred at 100° C. for 4 h. The reaction was quenched with NaHCO₃ (50 mL) and extracted with EtOAc (3×30 mL). The combined organic layers were washed with brine, dried over Na₂SO₄, and then concentrated in vacuo to give the crude, which was purified by silica gel column chromatography (eluent: DCM/MeOH=10/1) to give N5-((6-cyclopropylimidazo[1,2-a]pyridin-2-yl)methyl)-1,2,4-thiadiazole-3,5-diamine (200 mg, 71%) as a white solid. ESI-MS [M+H]+: 287.2.

Synthesis of rac-(1S*,2S*)-2-(3-chloropyridinyl)-N-(5-(((6-cyclopropylimidazo[1,2-a]pyridin-2-yl)methyl)amino)-1,2,4-thiadiazol-3-yl)cyclopropane-1-carboxamide (I-614). To a solution of rac-(1S*,2S*)-2-(3-chlorophenyl)cyclopropane-1-carboxylic acid (82 mg, 0.42 mmol) in DCM (5 ml) was added oxalyl dichloride (53 mg, 0.34 mmol) and DMF (3 drops) at 0° C. under N₂. After stirring at 0° C. for 3 h, the mixture was evaporated to give rac-(1S*,2S*)-2-(3-chlorophenyl)cyclopropane-1-carboxylic acid as a light yellow oil (80 mg, crude). This was dissolved in DCM (3 mL) then N5-((6-cyclopropylimidazo[1,2-a]pyridin-2-yl)methyl)-1,2,4-thiadiazole-3,5-diamine (80 mg, 0.28 mmol) and pyridine (1 mL) were added. The mixture was stirred at room temperature for 12 h. The reaction mixture was quenched with water (10 mL) and extracted with EtOAc (3×10 mL). The combined organic layers were washed with brine, dried over Na₂SO₄, and concentrated in vacuo to give the crude, which was purified by silica gel column chromatography (eluent: DCM/MeOH=10/1) to give rac-(1S*,2S*)-2-(3-chloropyridinyl)-N-(5-(((6-cyclopropylimidazo[1,2-a]pyridin-2-yl)methyl)amino)-1,2,4-thiadiazol-3-yl)cyclopropane-1-carboxamide (20 mg, 15%) as a white solid. ESI-MS [M+H]+: 466.1, ¹H NMR (400 MHz, DMSO) δ 10.90 (s, 1H), 8.87 (s, 1H), 8.40 (d, J=5.3 Hz, 1H), 8.32 (s, 1H), 7.72 (s, 1H), 7.64 (s, 1H), 7.39 (d, J=9.3 Hz, 1H), 7.33 (d, J=5.2 Hz, 1H), 6.98 (d, J=9.3 Hz, 1H), 4.53 (d, J=3.9 Hz, 2H), 2.58-2.55 (m, 2H), 1.96-1.86 (m, 1H), 1.48-1.42 (m, 2H), 0.94-0.88 (m, 2H), 0.72-0.60 (m, 2H).

### Example 615

### Synthesis of (1S,2S)-2-(3-chlorophenyl)-N-(5-(((6-cyclopropylimidazo[1,2-a]pyridin-2-yl)methyl)amino)-1,2,4-oxadiazol-3-yl)cyclopropane-1-carboxamide (I-615)

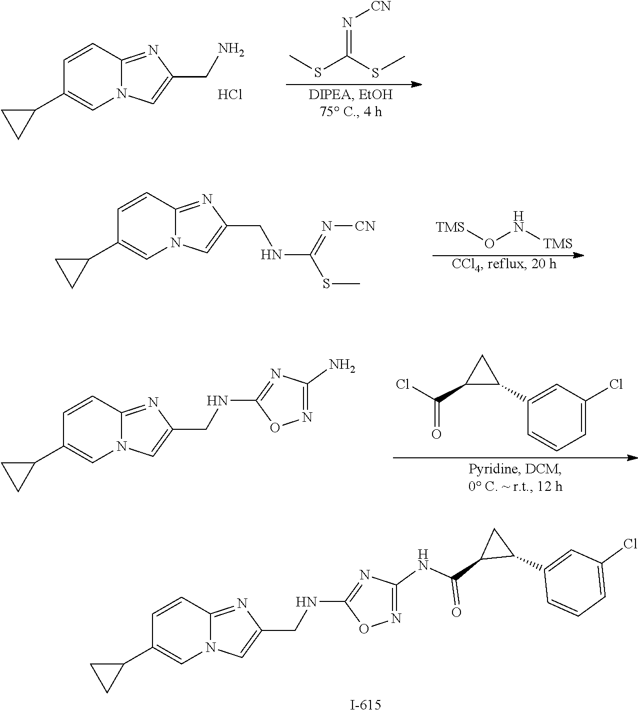

Synthesis of methyl (Z)—N′-cyano-N-((6-cyclopropylimidazo[1,2-a]pyridin-2-yl)methyl)carbamimidothioate. To a mixture of (6-cyclopropylimidazo[1,2-a]pyridin-2-yl)methanamine hydrochloride (450 mg, 2 mmol) and dimethyl cyanocarbonimidodithioate (438 mg, 3 mmol) in EtOH (20 mL) was added DIPEA (774 mg, 6 mmol) at room temperature and then was stirred at 75° C. for 4 h. The reaction was cooled to room temperature and concentrated in vacuo to give the crude, which was purified by silica gel column chromatography (eluent: DCM/MeOH=30/1) to give the N-(6-(((6-cyclopropyl-8-(3-methyl-2,4-dioxoimidazolidin-1-yl)imidazo[1,2-a]pyridin-2-yl)methyl-d2)amino)pyrimidin-4-yl)-2-(4-methylpyrimidin-2-yl)cyclopropane-1-carboxamide (450 mg, 9%) as a yellow solid. ESI-MS [M+H]+: 286.1.

Synthesis of N⁵-((6-cyclopropylimidazo[1,2-a]pyridin-2-yl)methyl)-1,2,4-oxadiazole-3,5-diamine. To a mixture of methyl (Z)—N′-cyano-N-((6-cyclopropylimidazo[1,2-a]pyridin-2-yl)methyl)carbamimidothioate (250 mg, 0.88 mmol) in CCl4 (4 mL) and 1,4-dioxane (4 mL) was added N,O-bis(trimethylsilyl)hydroxylamine (779 mg, 4.4 mmol). After stirring at 90° C. for 20 h, the reaction mixture was then cooled to room temperature and concentrated in vacuo to give the crude, which was purified by silica gel column chromatography (eluent: DCM/MeOH=30/1) to give the N⁵-((6-cyclopropylimidazo[1,2-a]pyridin-2-yl)methyl)-1,2,4-oxadiazole-3,5-diamine (150 mg, 63%) as a yellow oil. ESI-MS [M+H]+: 271.1.

Synthesis of (1S,2S)-2-(3-chlorophenyl)-N-(5-(((6-cyclopropylimidazo[1,2-a]pyridin-2-yl)methyl)amino)-1,2,4-oxadiazol-3-yl)cyclopropane-1-carboxamide (I-615). To a stirred solution of N5-((6-cyclopropylimidazo[1,2-a]pyridin-2-yl)methyl)-1,2,4-oxadiazole-3,5-diamine (90 mg, 0.33 mmol) in DCM (6 mL) was added pyridine (1 mL) and (1S,2S)-2-(3-chlorophenyl)cyclopropane-1-carbonyl chloride (140 mg, 0.66 mmol) at 0° C. After stirring at room temperature for 12 h, the reaction mixture was concentrated in vacuo to give the crude, which was purified with preparative TLC (eluent: DCM/MeOH=20/1) to give (1S,2S)-2-(3-chlorophenyl)-N-(5-(((6-cyclopropylimidazo[1,2-a]pyridin-2-yl)methyl)amino)-1,2,4-oxadiazol-3-yl)cyclopropane-1-carboxamide (7.3 mg, 5%) as a white solid. ESI-MS [M+H]⁺: 449.1. ¹H NMR (400 MHz, DMSO) δ 10.08 (s, 1H), 8.33 (s, 1H), 7.68 (s, 1H), 7.59 (t, J=9.4 Hz, 1H), 7.41-7.34 (m, 2H), 7.33-7.28 (m, 2H), 7.24-7.21 (m, 1H), 7.01-6.92 (m, 1H), 4.42 (d, J=5.2 Hz, 2H), 2.75-2.64 (m, 2H), 1.99-1.89 (m, 1H), 1.81-1.73 (m, 2H), 0.94-0.89 (m, 2H), 0.69-0.65 (m, 2H).

### Example 616

(1S,2S)-2-(3-chlorophenyl)-N-(6-((2R,4S)-2-(6-cyclopropylimidazo[1,2-a]pyridin-2-yl)-4-methoxypyrrolidin-1-yl)pyrimidin-4-yl)cyclopropane-1-carboxamide (I-616)

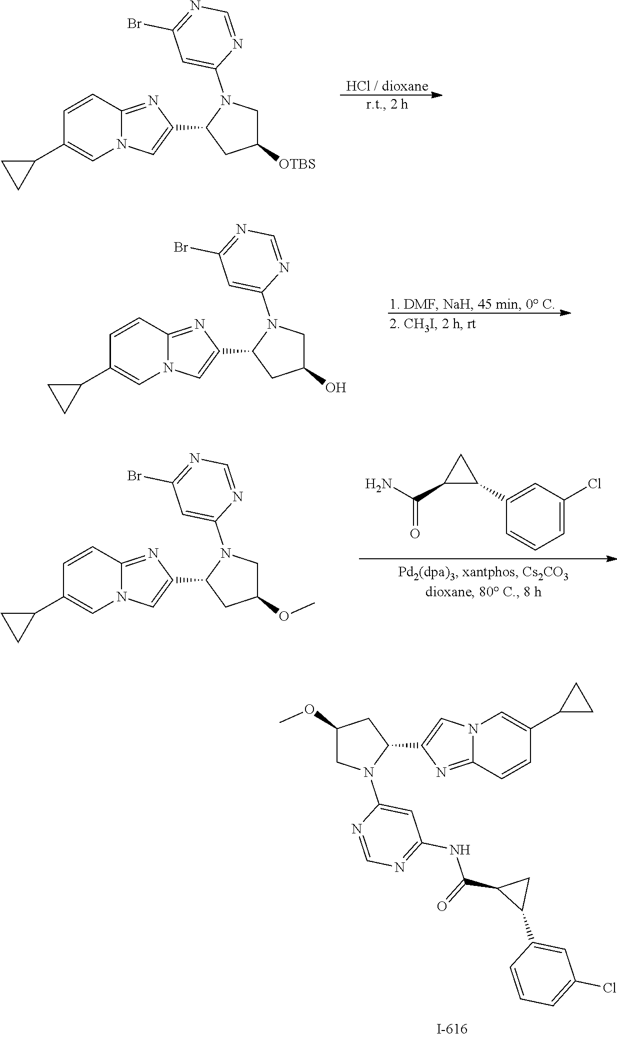 [SMILES: CC(C)(C)[Si](C)(C)O[C@H]1C[C@H](c2cn3cc(C4CC4)ccc3n2)N(c2cc(Br)ncn2)C1 ; O[C@H]1C[C@H](c2cn3cc(C4CC4)ccc3n2)N(c2cc(Br)ncn2)C1 ; CO[C@H]1C[C@H](c2cn3cc(C4CC4)ccc3n2)N(c2cc(Br)ncn2)C1 ; NC(=O)[C@H]1C[C@@H]1c1cccc(Cl)c1 ; CO[C@H]1C[C@H](c2cn3cc(C4CC4)ccc3n2)N(c2cc(NC(=O)[C@H]3C[C@@H]3c3cccc(Cl)c3)ncn2)C1]

Synthesis of (3S,5R)-1-(6-bromopyrimidin-4-yl)-5-(6-cyclopropylimidazo[1,2-a]pyridin-2-yl)pyrrolidin-3-ol. A mixture of 2-((2R,4S)-1-(6-bromopyrimidin-4-yl)-4-((tert-butyldimethylsilyl)oxy)pyrrolidin-2-yl)-6-cyclopropylimidazo[1,2-a]pyridine (200 mg, 0.39 mmol) in HCl (4M solution in dioxane, 5 mL) was stirred at room temperature for 1 h. The reaction mixture was concentrated in vacuo to give the crude, which was purified by preparative HPLC to give (3S,5R)-1-(6-bromopyrimidin-4-yl)-5-(6-cyclopropylimidazo[1,2-a]pyridin-2-yl)pyrrolidin-3-ol (170 mg, 43%) as a white solid. ESI-MS [M+H]⁺: 400.1.

Synthesis of 2-((2R,4S)-1-(6-bromopyrimidin-4-yl)-4-methoxypyrrolidin-2-yl)-6-cyclopropylimidazo[1,2-a]pyridine. To mixture of (3S,5R)-1-(6-bromopyrimidin-4-yl)-5-(6-cyclopropylimidazo[1,2-a]pyridin-2-yl)pyrrolidin-3-ol (170 mg, 0.425 mmol) in dry DMF (8.0 mL) was added NaH (21 mg, 0.51 mmol, 60% dispersion in mineral oil) at 0° C. After stirring at 0° C. for 45 min, a solution of Mel (125 mg, 0.85 mmol) in dry THF (1.7 mL) was added and the resulting mixture was stirred at room temperature for another 2 h. The reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by silica gel column chromatography (eluent: DCM/MeOH=20/1) to give 2-((2R,4S)-1-(6-bromopyrimidin-4-yl)-4-methoxypyrrolidin-2-yl)-6-cyclopropylimidazo[1,2-a]pyridine (100 mg, 57%) as a yellow solid. ESI-MS [M+H]⁺: 414.1.

Synthesis of (1S,2S)-2-(3-chlorophenyl)-N-(6-((2R,4S)-2-(6-cyclopropylimidazo[1,2-a]pyridin-2-yl)-4-methoxypyrrolidin-1-yl)pyrimidin-4-yl)cyclopropane-1-carboxamide (I-616). To a mixture of 2-((2R,4S)-1-(6-bromopyrimidin-4-yl)-4-methoxypyrrolidin-2-yl)-6-cyclopropyl-imidazo[1,2-a]pyridine (80 mg, 0.19 mmol), (1S,2S)-2-(3-chlorophenyl)cyclopropane-1-carboxamide (38 mg, 0.19 mmol) and Cs₂CO₃ (186 mg, 0.57 mmol) in 1,4-dioxane (5 mL) was added Pd₂(dba)₃ (18 mg, 0.019 mmol) and Xantphos (23 mg, 0.038 mmol). The reaction mixture was stirred at 80° C. for 1 h under N₂. The reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)-2-(3-chlorophenyl)-N-(6-((2R,4S)-2-(6-cyclopropylimidazo[1,2-a]pyridin-2-yl)-4-methoxypyrrolidin-1-yl)pyrimidin-4-yl)cyclopropane-1-carboxamide (33 mg, 33%) as a yellow solid. ESI-MS: [M+H]+, 529.2. ¹H NMR (400 MHz, DMSO) δ 10.68-10.59 (m, 1H), 8.25 (s, 2H), 7.59 (s, 1H), 7.37 (d, J=9.2 Hz, 1H), 7.32-7.28 (m, 1H), 7.26-7.21 (m, 2H), 7.17-7.12 (m, 2H), 6.95 (d, J=9.2 Hz, 1H), 4.96 (s, 1H), 4.19 (s, 1H), 3.84-3.79 (m, 2H), 3.26 (s, 3H), 2.40-2.31 (m, 4H), 1.91-1.88 (m, 1H), 1.39-1.33 (m, 2H), 0.91-0.87 (m, 2H), 0.65-0.63 (m, 2H).

### Example 617

### Synthesis of rac-(1S*,2S*)—N-(5-(((6-cyclopropyl-8-(3-methyl-2,4-dioxoimidazolidin-1-yl) imidazo[1,2-a]pyridin-2-yl)methyl)amino)pyridazin-3-yl)-2-(4-methylpyrimidin-2-yl)cyclo propane-1-carboxamide (I-617)

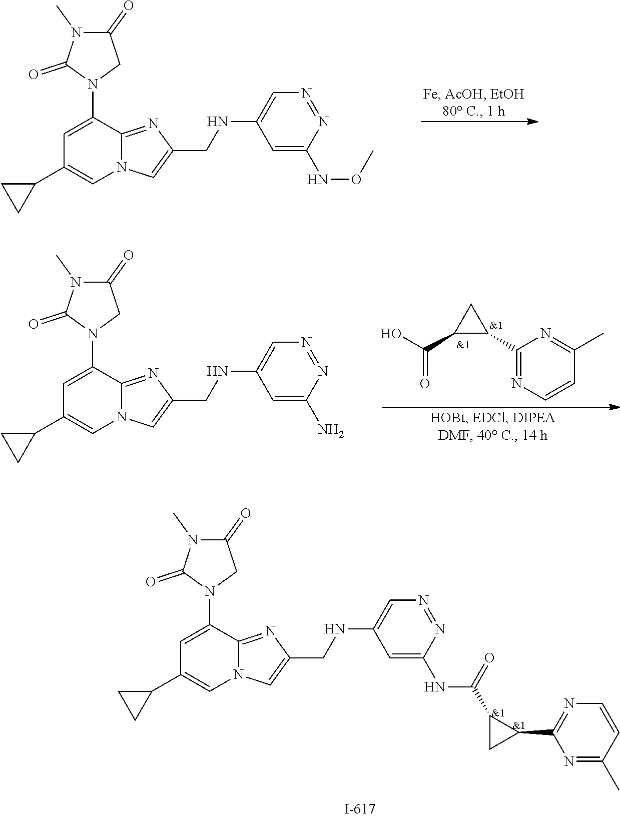 [SMILES: CONc1cc(NCc2cn3cc(C4CC4)cc(N4CC(=O)N(C)C4=O)c3n2)cnn1 ; CN1C(=O)CN(c2cc(C3CC3)cn3cc(CNc4cnnc(N)c4)nc23)C1=O ; Cc1ccnc([C@H]2C[C@@H]2C(=O)O)n1 ; Cc1ccnc([C@H]2C[C@@H]2C(=O)Nc2cc(NCc3cn4cc(C5CC5)cc(N5CC(=O)N(C)C5=O)c4n3)cnn2)n1]

Synthesis of 1-(2-(((6-aminopyridazin-4-yl)amino)methyl)-6-cyclopropylimidazo[1,2-a]pyridin-8-yl)-3-methylimidazolidine-2,4-dione. To a mixture of 1-(6-cyclopropyl-2-(((6-(methoxyamino)pyridazin-4-yl)amino)methyl)imidazo[1,2-a]pyridin-8-yl)-3-methylimidazolidine-2,4-dione (800 mg, 1.92 mmol) in EtOH (5 mL) was added Fe (528 mg, 9.6 mmol) and AcOH (4 mL) at room temperature. After stirring at 80° C. for 1 h, the reaction mixture was poured into water (50 mL) and extracted with EtOAc (3×50 mL). The combined organics were washed with brine (50 mL) then dried over Na₂SO₄. The reaction mixture was concentrated in vacuo and purified by silica gel column chromatography (eluent: PE/EtOAc=5/1) to give 1-(2-(((6-aminopyridazin-4-yl)amino)methyl)-6-cyclopropylimidazo[1,2-a]pyridin-8-yl)-3-methylimidazolidine-2,4-dione (600 mg, 80%) as a colorless oil. ESI-MS [M+H]+: 393.1

Synthesis of rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclopropane-1-carboxamide (I-617). To a stirred solution of 1-(2-(((6-aminopyridazin-4-yl)amino)methyl)-6-cyclopropylimidazo[1,2-a]pyridin-8-yl)-3-methylimidazolidine-2,4-dione (100 mg, 0.26 mmol) in DMF (5 mL) was added rac-(1S*,2S*)-2-(4-methylpyrimidin-2-yl)cyclopropane-1-carboxylic acid (44 mg, 0.26 mmol), EDCI (146 mg, 0.52 mmol), HOBt (102 mg, 0.52 mmol), and DIPEA (164.6 mg, 2.6 mmol). The mixture was stirred at 40° C. for 14 h. The reaction was concentrated in vacuo to give the crude product, which was purified by preparative HPLC to give rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclopropane-1-carboxamide (43.4 mg, 30%) as a white solid. ESI-MS [M+H]+: 553.1. ¹H NMR (400 MHz, DMSO) δ 10.87 (s, 1H), 8.49 (d, J=5.1 Hz, 1H), 8.37 (d, J=2.5 Hz, 1H), 8.23 (s, 1H), 7.73 (s, 1H), 7.52-7.50 (m, 2H), 7.35-7.31 (m, 1H), 7.17 (d, J=5.1 Hz, 1H), 4.90 (s, 2H), 4.38 (d, J=5.5 Hz, 2H), 2.95 (s, 3H), 2.64-2.57 (m, 2H), 2.38 (s, 3H), 1.95-1.88 (m, J=13.3 Hz, 1H), 1.50-1.44 (m, J=13.1 Hz, 2H), 0.93-0.89 (m, 2H), 0.64-0.60 (m, J=4.7 Hz, 2H).

### Example 618

### Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-(trifluoromethyl)pyridin-2-yl)-2-(4-methylpyrimidin-2-yl)cyclopropane-1-carboxamide (I-618)

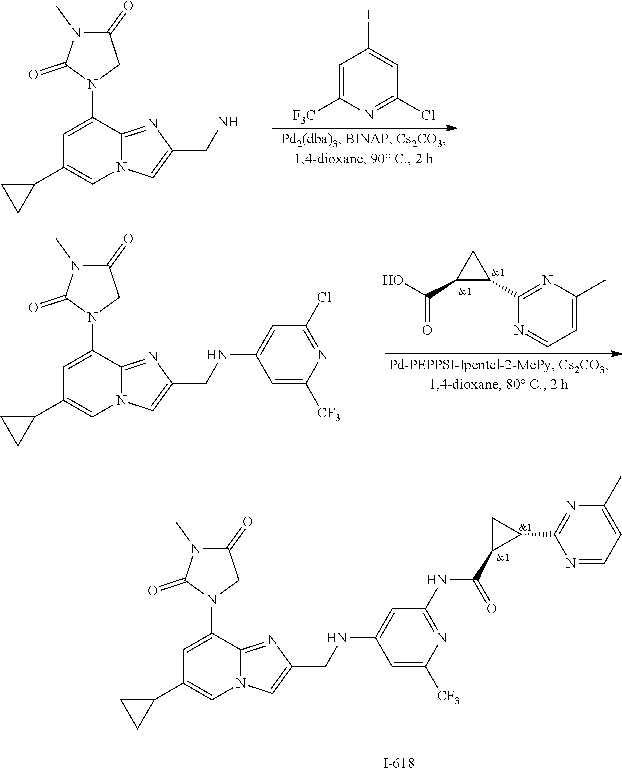

Synthesis of 1-(2-(((2-chloro-6-(trifluoromethyl)pyridin-4-yl)amino)methyl)-6-cyclopropylimidazo[1,2-a]pyridin-8-yl)-3-methylimidazolidine-2,4-dione. To a mixture of 2-chloro-4-iodo-6-(trifluoromethyl)pyridine (384 mg, 1.25 mmol), 1-(2-(aminomethyl)-6-cyclopropylimidazo[1,2-a]pyridin-8-yl)-3-methylimidazolidine-2,4-dione (299.0 mg, 1.0 mmol) and Cs₂CO₃ (978 mg, 3.0 mmol) in 1,4-dioxane (20 mL) were added Pd₂(dba)₃ (45.8 mg, 0.05 mmol) and BINAP (62.3 mg, 0.1 mmol). The reaction mixture was stirred at 90° C. for 2 h under N₂ and cooled to room temperature. Then the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give 1-(2-(((2-chloro-6-(trifluoromethyl)pyridin-4-yl)amino)methyl)-6-cyclopropylimidazo[1,2-a]pyridin-8-yl)-3-methylimidazolidine-2,4-dione (200 mg, 42%) as a yellow solid. ESI-MS [M+H]⁺: 479.2.

Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-(trifluoromethyl)pyridin-2-yl)-2-(4-methylpyrimidin-2-yl)cyclopropane-1-carboxamide (I-618). To a mixture of 1-(2-(((2-chloro-6-(trifluoromethyl)pyridin-4-yl)amino)methyl)-6-cyclopropyl-imidazo[1,2-a]pyridin-8-yl)-3-methylimidazolidine-2,4-dione (65 mg, 0.14 mmol), rac-(1S*,2S*)-2-(4-methylpyrimidin-2-yl)cyclopropane-1-carboxamide (24 mg, 0.14 mmol) and Cs₂CO₃ (137 mg, 0.42 mmol) in 1,4-dioxane (6 mL) was added Pd-PEPPSI-IpentCl-2-MePy (12 mg, 0.014 mmol). The reaction mixture was stirred at 80° C. for 2 h under N₂ and cooled to room temperature. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=15/1) to give rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-(trifluoromethyl)pyridin-2-yl)-2-(4-methylpyrimidin-2-yl)cyclopropane-1-carboxamide (40 mg, 46%) as a yellow solid. ESI-MS [M+H]⁺: 620.2. ¹H NMR (400 MHz, DMSO) δ 10.82 (s, 1H), 8.52 (d, J=5.1 Hz, 1H), 8.27 (s, 1H), 7.76 (s, 1H), 7.69-7.65 (m, 2H), 7.36 (d, J=1.4 Hz, 1H), 7.20 (d, J=5.1 Hz, 1H), 6.84 (d, J=1.7 Hz, 1H), 4.92 (s, 2H), 4.44 (d, J=5.6 Hz, 2H), 2.98 (s, 3H), 2.62-2.60 (m, 2H), 2.41 (s, 3H), 1.96-1.91 (m, 1H), 1.60-1.39 (m, 2H), 1.04-0.83 (m, 2H), 0.74-0.52 (m, 2H).

### Example 619

### Synthesis of rac-(1S*,2S*)—N-(5-(((6-cyclopropylimidazo[1,2-a]pyridin-2-yl)methyl)amino)-2-(methylsulfonyl)phenyl)-2-(4-methylpyrimidin-2-yl)cyclopropane-1-carboxamide (I-619)

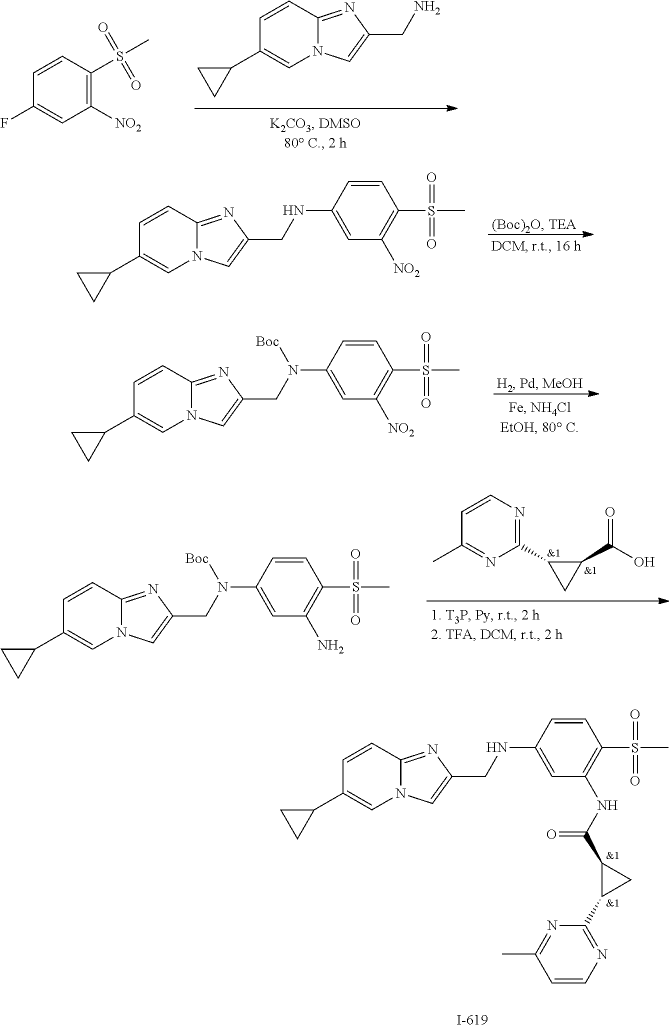

Synthesis of N-((6-cyclopropylimidazo[1,2-a]pyridin-2-yl)methyl)-4-(methylsulfonyl)-3-nitroaniline. To a mixture of 4-fluoro-1-(methylsulfonyl)-2-nitrobenzene (430 mg, 1.96 mmol) and (6-cyclopropylimidazo[1,2-a]pyridin-2-yl)methanamine (366 mg, 2.16 mmol) in DMSO (10 mL) was added K₂CO₃ (676 mg, 4.9 mmol) and the mixture was stirred at 80° C. for 2 h under N₂. After cooling to 25° C., water (50 mL) was added and the mixture was extracted with EtOAc (3×30 mL). The combined organic layers were washed with brine (60 mL), dried over anhydrous Na₂SO₄, and concentrated in vacuo to give N-((6-cyclopropylimidazo[1,2-a]pyridin-2-yl)methyl)-4-(methylsulfonyl)-3-nitroaniline (740 mg, crude) as a yellow oil. ESI-MS [M+H]+: 387.1.

Synthesis of tert-butyl ((6-cyclopropylimidazo[1,2-a]pyridin-2-yl)methyl)(4-(methylsulfonyl)-3-nitrophenyl)carbamate. To a mixture of N-((6-cyclopropylimidazo[1,2-a]pyridin-2-yl)methyl)-4-(methylsulfonyl)-3-nitroaniline (740 mg, 1.9 mmol) in DCM (30 mL) were added di-tert-butyl dicarbonate (828 mg, 3.8 mmol), DMAP (23 mg, 0.19 mmol), and TEA (576 mg, 5.7 mmol). After stirring at 25° C. for 16 h under N₂, water (50 mL) was added to the mixture and extracted with EtOAc (3×30 mL). The combined organic layers were washed with brine (60 mL), dried over anhydrous Na₂SO₄, and concentrated in vacuo to give tert-butyl ((6-cyclopropylimidazo[1,2-a]pyridin-2-yl)methyl)(4-(methylsulfonyl)-3-nitrophenyl)carbamate (900 mg, 60%) as a yellow solid. ESI-MS [M+H]⁺: 487.2.

Synthesis of tert-butyl (3-amino-4-(methylsulfonyl)phenyl)((6-cyclopropylimidazo[1,2-a]pyridin-2-yl)methyl)carbamate. To a mixture of tert-butyl ((6-cyclopropylimidazo[1,2-a]pyridin-2-yl)methyl)(4-(methylsulfonyl)-3-nitrophenyl)carbamate (900 mg, 1.85 mmol) in MeOH (10 mL) and THF (2 mL) was added Pd/C (200 mg) and the mixture was stirred at 25° C. for 2 h under H₂. The reaction mixture was filtered through Celite® and the filter cake was washed with DCM/MeOH (10/1, 20 mL). The filtrate was concentrated in vacuo and the resulting residue was mixed with EtOH (20 mL) and NH₄Cl (1.08 g, 20 mmol). The mixture was then heated to 80° C. and Fe powder (311 mg, 5.55 mmol) was added. The mixture was stirred at 80° C. for 20 min under N₂, then filtered through Celite® and the filter cake was washed with DCM/MeOH (10/1, 20 mL). The filtrate was concentrated in vacuo to give the crude, which was purified by silica gel column chromatography (eluent: DCM/MeOH=50/1) to give tert-butyl (3-amino-4-(methylsulfonyl)phenyl)((6-cyclopropylimidazo[1,2-a]pyridin-2-yl)methyl)carbamate (450 mg, 53%) as a yellow solid. ESI-MS [M+H]+: 457.2.

Synthesis of rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate. To a mixture of tert-butyl (3-amino-4-(methylsulfonyl)phenyl)((6-cyclopropylimidazo[1,2-a]pyridin-2-yl)methyl)carbamate (200 mg, 0.44 mmol) and rac-(1S*,2S*)-2-(4-methylpyrimidin-2-yl)cyclopropane-1-carboxylic acid (78 mg, 0.44 mmol) in pyridine (5 mL) was added T₃P (2.1 g, 6.6 mmol) dropwise at 25° C. under N₂ for 3 h under N₂. Water (20 mL) was added, and the mixture was extracted with EtOAc (3×20 mL). The combined organic layers were washed with brine (10 mL), dried over anhydrous Na₂SO₄ and concentrated to give the crude product, which was purified by preparative TLC (eluent: DCM/MeOH=15/1) to give rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate (200 mg, 74%) as a yellow solid. ESI-MS [M+H]+: 617.3.

Synthesis of rac-(1S*,2S*)—N-(5-(((6-cyclopropylimidazo[1,2-a]pyridin-2-yl)methyl)amino)-2-(methylsulfonyl)phenyl)-2-(4-methylpyrimidin-2-yl)cyclopropane-1-carboxamide (I-619). To a solution of give rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate (50 mg, 0.081 mmol) in DCM (3 mL) was added TFA (1 mL). The mixture was stirred at room temperature for 2 h. The reaction mixture was concentrated and neutralized by adding NH3 (5.0 mL, 4 N in MeOH, 20 mmol), then concentrated to give the crude product, which was purified by Prep-TLC (eluent: DCM/MeOH=10/1) to give rac-(1S*,2S*)—N-(5-(((6-cyclopropylimidazo[1,2-a]pyridin-2-yl)methyl)amino)-2-(methylsulfonyl)phenyl)-2-(4-methylpyrimidin-2-yl)cyclopropane-1-carboxamide (25 mg, 59.7%) as a yellow solid. ESI-MS [M+H]+: 517.2. 1H NMR (400 MHz, DMSO) δ 9.84 (s, 1H), 8.54 (d, J=5.1 Hz, 1H), 8.32 (s, 1H), 7.67 (s, 1H), 7.52 (d, J=8.9 Hz, 1H), 7.39 (d, J=9.3 Hz, 1H), 7.36-7.29 (m, 2H), 7.22 (d, J=5.1 Hz, 1H), 6.97 (dd, J=9.3, 1.6 Hz, 1H), 6.55 (dd, J=8.8, 2.1 Hz, 1H), 4.39 (d, J=5.7 Hz, 2H), 3.09 (s, 3H), 2.65-2.57 (m, 1H), 2.43 (s, 3H), 2.34-2.33 (m, 1H), 1.95-1.88 (m, 1H), 1.56-1.50 (m, 2H), 0.94-0.88 (m, 2H), 0.69-0.64 (m, 2H).

### Example 620

### Synthesis of rac-(1S*,2S*)—N-(6-((6-cyclopropyl-8-(3-methyl-2,4-dioxoimidazolidin-1-yl)imidazo[1,2-a]pyridin-2-yl)methyl)pyrimidin-4-yl)-2-(4-methylpyrimidin-2-yl)cyclopropane-1-carboxamide (I-620)

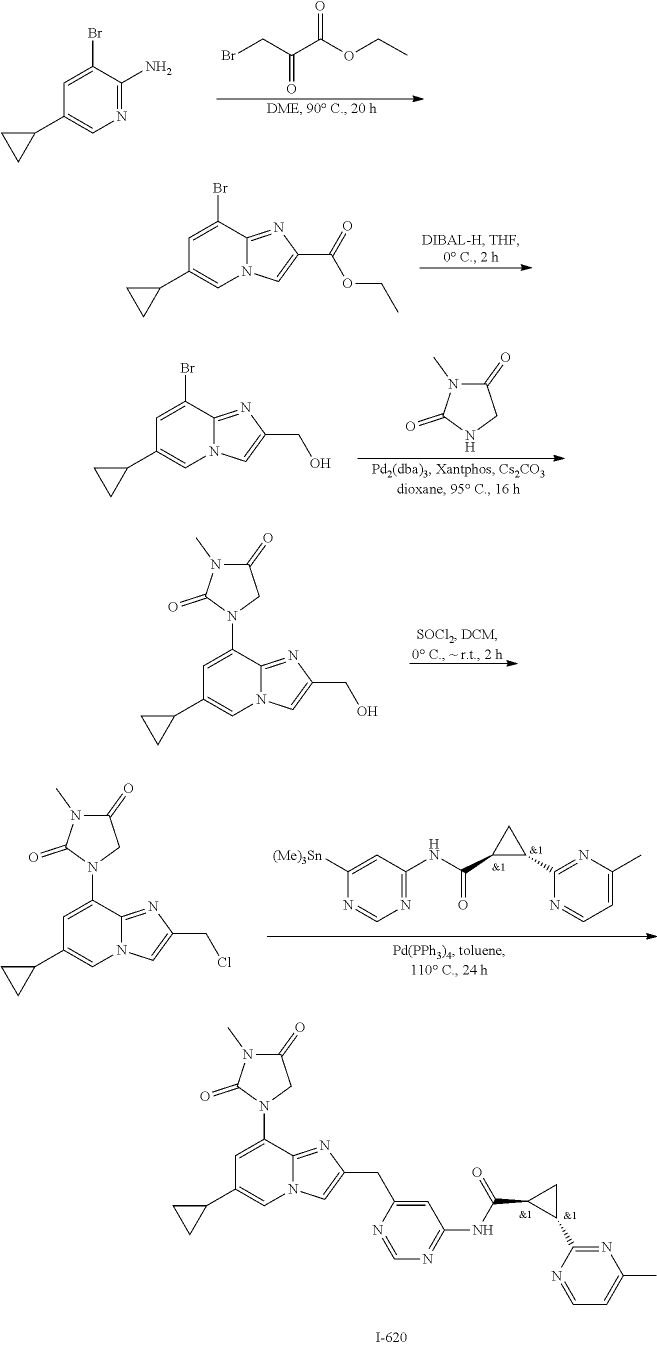

Synthesis of ethyl 8-bromo-6-cyclopropylimidazo[1,2-a]pyridine-2-carboxylate. A mixture of 3-bromo-5-cyclopropylpyridin-2-amine (2.5 g, 11.8 mmol) and ethyl 3-bromo-2-oxopropanoate (2.7 g, 14.2 mmol) in DME (50 mL) was stirred at 90° C. for 20 h. After cooling to room temperature, the reaction mixture was concentrated in vacuo and diluted with EtOAc (150 mL). The organic extract was washed with saturated aq·NaHCO₃ (2×100 mL), dried over Na₂SO₄, concentrated in vacuo and purified by silica gel chromatography (EtOAc/PE=1/10) to give product (2.0 g, 56%) as a white solid. ESI-MS (M+H)+: 309.0.

Synthesis of (8-bromo-6-cyclopropylimidazo[1,2-a]pyridin-2-yl)methanol. To a mixture of ethyl 8-bromo-6-cyclopropylimidazo[1,2-a]pyridine-2-carboxylate (2.0 g, 11.8 mmol) in THF (50 mL) was added DIBAL-H (1M solution in THF, 19.5 mL, 19.5 mmol) at 0° C. After stirring at room temperature for 2 h, the reaction mixture was cooled to 0° C. and quenched by addition of water (1.0 mL), followed by 15% aq·NaOH (1.0 mL). After stirring at room temperature for 15 min, the precipitated solid was removed by filtration. The filtrate was concentrated to dryness to give crude product, which was purified by silica gel chromatography (DCM/MeOH=20/1) to give (8-bromo-6-cyclopropylimidazo[1,2-a]pyridin-2-yl)methanol (1.3 g, 76%) as a white solid. ESI-MS (M+H)+: 267.1

Synthesis of 1-(6-cyclopropyl-2-(hydroxymethyl)imidazo[1,2-a]pyridin-8-yl)-3-methylimidazolidine-2,4-dione. To a mixture of (8-bromo-6-cyclopropylimidazo[1,2-a]pyridin-2-yl)methanol (1.3 g, 4.9 mmol), 3-methylimidazolidine-2,4-dione (1.12 g, 9.8 mmol), and Cs₂CO₃ (4.8 g, 14.7 mmol) in dioxane (50 mL) were added Pd₂(dba)₃ (455 mg, 0.5 mmol) and Xantphos (286 mg, 1.1 mmol). The reaction mixture was stirred at 90° C. for 16 h under N₂ and cooled to room temperature. The reaction mixture was filtered and the filter cake was washed with DCM/MeOH (10/1, 30 mL). The filtrate was concentrated in vacuo to give the crude, which was purified by silica gel column chromatography (eluent: DCM/MeOH=50/1˜20/1) to give 1-(6-cyclopropyl-2-(hydroxymethyl)imidazo[1,2-a]pyridin-8-yl)-3-methylimidazolidine-2,4-dione (530 mg, 36%) as a yellow solid. ESI-MS [M+H]+: 301.1.

Synthesis of 1-(2-(chloromethyl)-6-cyclopropylimidazo[1,2-a]pyridin-8-yl)-3-methylimidazolidine-2,4-dione. To a solution of 1-(6-cyclopropyl-2-(hydroxymethyl)imidazo[1,2-a]pyridin-8-yl)-3-methylimidazolidine-2,4-dione (350 mg, 1.82 mmol) in DCM (10 ml) was added SOCl₂ (0.5 mL) at 0° C. After stirring at room temperature for 2 h, the reaction mixture was concentrated in vacuo to give the crude, which was purified by silica gel column chromatography (PE/EtOAc from 0 to 50%) to afford 1-(2-(chloromethyl)-6-cyclopropylimidazo[1,2-a]pyridin-8-yl)-3-methylimidazolidine-2,4-dione (280 mg, 75%) as a brown oil. ESI-MS [M+H]+: 319.1.

Synthesis of rac-(1S*,2S*)-2-(4-methylpyrimidin-2-yl)-N-(6-(trimethylstannyl)pyrimidin-4-yl)cyclopropane-1-carboxamide. A mixture of rac-(1S*,2S*)—N-(6-chloropyrimidin-4-yl)-2-(4-methylpyrimidin-2-yl)cyclopropane-1-carboxamide (200 mg, 0.69 mmol), 1,1,1,2,2,2-hexamethyldistannane (227 mg, 104 mmol), and Pd(PPh₃)₄ (81 mg, 0.07 mmol) in toluene (10 mL) was stirred at 110° C. for 16 h. The reaction mixture was cooled to room temperature and filtered. The filtrate was concentrated in vacuo to rac-(1S*,2S*)-2-(4-methylpyrimidin-2-yl)-N-(6-(trimethylstannyl)pyrimidin-4-yl)cyclopropane-1-carboxamide (350 mg crude), which was used into next step without any purification. ESI-MS [M+H]+: 402.0

Synthesis of rac-(1S*,2S*)—N-(6-((6-cyclopropyl-8-(3-methyl-2,4-dioxoimidazolidin-1-yl)imidazo[1,2-a]pyridin-2-yl)methyl)pyrimidin-4-yl)-2-(4-methylpyrimidin-2-yl)cyclopropane-1-carboxamide (I-620). A mixture of rac-(1S*,2S*)-2-(4-methylpyrimidin-2-yl)-N-(6-(trimethylstannyl)pyrimidin-4-yl)cyclopropane-1-carboxamide (150 mg crude), 1-(2-(chloromethyl)-6-cyclopropylimidazo[1,2-a]pyridin-8-yl)-3-methylimidazolidine-2,4-dione (114 mg, 0.36 mmol), and Pd (PPh₃)₄ (46 mg, 0.04 mmol) in toluene (10 mL) was stirred at 110° C. for 24 h. The reaction mixture was cooled to room temperature and filtered. The filtrate was concentrated in vacuo to provide crude product, which was purified by preparative HPLC to give rac-(1S*,2S*)—N-(6-((6-cyclopropyl-8-(3-methyl-2,4-dioxoimidazolidin-1-yl)imidazo[1,2-a]pyridin-2-yl)methyl)pyrimidin-4-yl)-2-(4-methylpyrimidin-2-yl)cyclopropane-1-carboxamide (17 mg, 9%). ESI-MS [M+H]+: 538.1, ¹H NMR (400 MHz, DMSO) δ 11.20 (s, 1H), 8.76 (d, J=1.1 Hz, 1H), 8.52 (d, J=5.1 Hz, 1H), 8.26 (d, J=1.1 Hz, 1H), 8.16 (s, 1H), 7.75 (s, 1H), 7.34 (d, J=1.5 Hz, 1H), 7.21 (d, J=5.1 Hz, 1H), 4.88 (d, J=1.4 Hz, 2H), 4.14 (s, 2H), 2.97 (s, 3H), 2.64 (dd, J=6.9, 4.6 Hz, 1H), 2.58-2.55 (m, 1H), 2.41 (s, 3H), 1.96 (ddd, J=13.4, 8.3, 5.0 Hz, 1H), 1.58-1.50 (m, 2H), 0.98-0.92 (m, 2H), 0.66 (dt, J=6.4, 4.5 Hz, 2H)

### Example 621

### Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide (I-621)

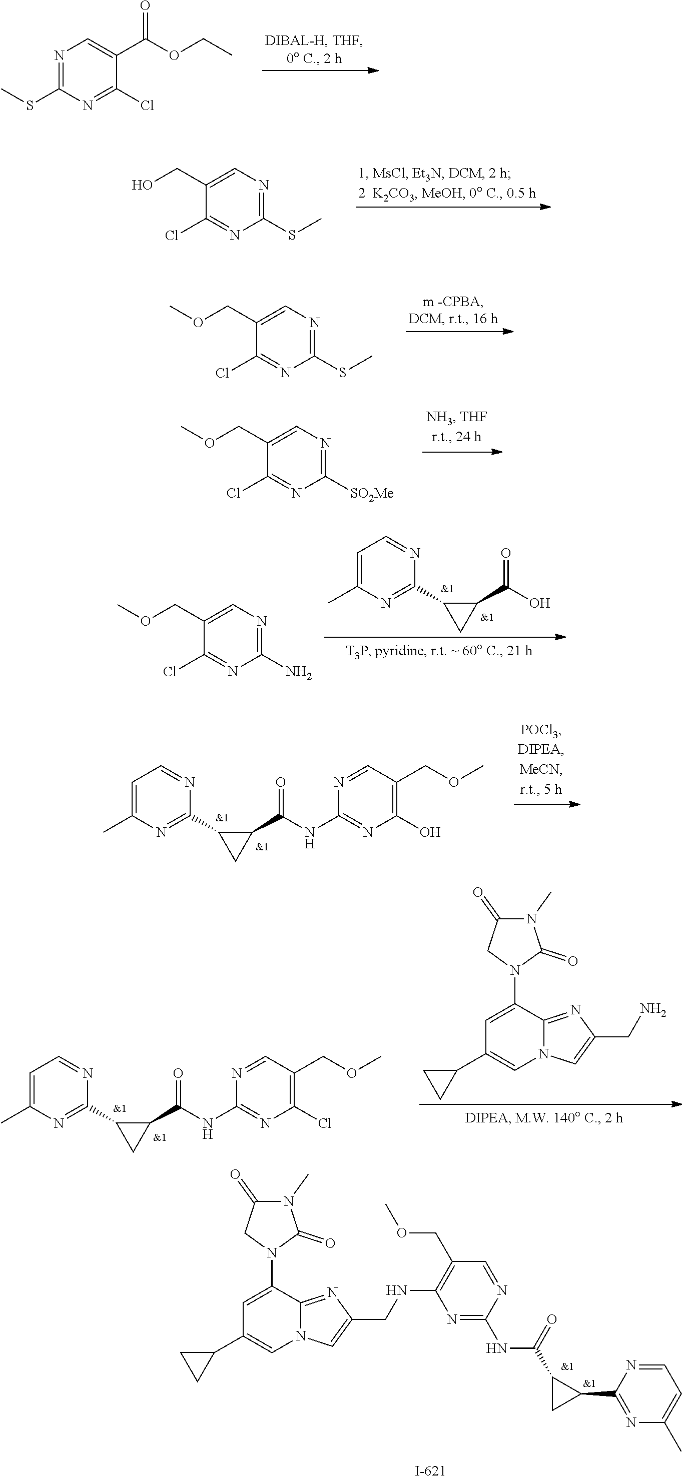

Synthesis of (4-chloro-2-(methylthio)pyrimidin-5-yl)methanol. To a solution of ethyl 4-chloro-2-(methylthio)pyrimidine-5-carboxylate (3 g, 13 mmol) in THF (30 mL) was added DIBAL-H (39 mL, 39 mmol) at 0° C., then the reaction mixture was stirred at 0° C. for 2 h under N₂. The reaction was quenched with water (30 mL) and extracted with EtOAc (3×30 mL). The combined organic layers were washed with brine (30 mL), dried over anhydrous Na₂SO₄, and concentrated in vacuo to give the crude product, which was purified by silica gel column chromatography (eluent:PE/EtOAc=1/1) to give (4-chloro-2-(methylthio)pyrimidin-5-yl)methanol (1.5 g, 61%) as a white solid. ESI-MS [M+H]+: 191.1.

Synthesis of 4-chloro-5-(methoxymethyl)-2-(methylthio)pyrimidine. To a solution of (4-chloro-2-(methylthio)pyrimidin-5-yl)methanol (500 mg, 2.63 mmol) in DCM (15 mL) was added Et₃N (797 mg, 7.89 mmol) and methanesulfonyl chloride (449 mg, 3.94 mmol) at 0° C., then the solution was stirred at room temperature for 2 h. Water (50 mL) was added and the mixture was extracted with EtOAc (3×30 mL). The combined organic layers were washed with brine (60 mL), dried over anhydrous Na₂SO₄, and concentrated in vacuo to give the crude (4-chloro-2-(methylthio)pyrimidin-5-yl)methyl methanesulfonate (500 mg, crude), which was used in the next step without purification. To a solution of (4-chloro-2-(methylthio)pyrimidin-5-yl)methyl methanesulfonate (500 mg, 0.26 mmol) in MeOH (10 mL) was added K₂CO₃ (258 mg, 1.87 mmol) at 0° C., then the solution was stirred at 0° C. for 30 min. Water (50 mL) was added and the mixture was extracted with EtOAc (3×30 mL). The combined organic layers were washed with brine (60 mL), dried over anhydrous Na₂SO₄, and concentrated in vacuo to give the crude, which was purified by column chromatography (eluent:PE/EtOAc=4/1) to give the product 4-chloro-5-(methoxy methyl)-2-(methylthio)pyrimidine (300 mg, 56%) as a colorless oil. ESI-MS [M+H]+: 205.1.

Synthesis of 4-chloro-5-(methoxymethyl)-2-(methylsulfonyl)pyrimidine. To a solution of 4-chloro-5-(methoxymethyl)-2-(methylthio)pyrimidine (400 mg, 1.96 mmol) in DCM (20 mL) was added m-CPBA (1.01 g, 5.88 mmol) and the reaction mixture was stirred at room temperature for 16 h. The reaction was quenched with saturated aq·Na₂S₂O₃ (30 mL) and saturated aq·NaHCO₃ (30 mL), then extracted with DCM (3×20 ml). The combined organic layers were washed with brine (30 mL), dried over anhydrous Na₂SO₄, and concentrated in vacuo to give the crude, which was purified by column chromatography (eluent:PE:EtOAc=1:1) to give 4-chloro-5-(methoxymethyl)-2-(methylsulfonyl)pyrimidine (380 mg, 82%) as a white solid. ESI-MS [M+H]+: 237.1.

Synthesis of 4-chloro-5-(methoxymethyl)pyrimidin-2-amine. A mixture of 4-chloro-5-(methoxymethyl)-2-(methylsulfonyl)pyrimidine (320 mg, 1.35 mmol) in ammonia solution (2M in THF, 40 mL) was stirred at room temperature for 24 h. The mixture was concentrated in vacuo to give the crude, which was purified by column chromatography (eluent: EtOAc/PE=8/1) to give 4-chloro-5-(methoxymethyl)pyrimidin-2-amine (170 mg, 73%) as a white solid. ESI-MS [M+H]+: 174.1.

Synthesis of rac-(1S*,2S*)—N-(4-hydroxy-5-(methoxymethyl)pyrimidin-2-yl)-2-(4-methylpyrimidin-2-yl)cyclopropane-1-carboxamide. To a solution of 4-chloro-5-(methoxymethyl)pyrimidin-2-amine (50 mg, 0.289 mmol) in pyridine (10 mL) was added rac-(1S*,2S*)-2-(4-methylpyrimidin-2-yl)cyclopropane-1-carboxylic acid (51 mg, 0.289 mmol) and T3P (1.84 g, 2.89 mmol, 50% in EtOAc). After stirring at 60° C. for 21 h under N₂, water (10 mL) was added and the mixture was extracted with EtOAc (3×20 mL). The combined organic layers were washed with brine (30 mL), dried over anhydrous Na₂SO₄, and concentrated in vacuo to give the crude, which was purified by column chromatography (eluent: DCM/MeOH=20/1) to give rac-(1S*,2S*)—N-(4-hydroxy-5-(methoxymethyl)pyrimidin-2-yl)-2-(4-methylpyrimidin-2-yl)cyclopropane-1-carboxamide (50 mg, 55%) as a white solid. ESI-MS [M+H]+: 316.1

Synthesis of rac-(1S*,2S*)—N-(4-chloro-5-(methoxymethyl)pyrimidin-2-yl)-2-(4-methylpyrimidin-2-yl)cyclopropane-1-carboxamide. To a solution of rac-(1S*,2S*)—N-(4-hydroxy-5-(methoxymethyl)pyrimidin-2-yl)-2-(4-methylpyrimidin-2-yl)cyclopropane-1-carboxamide (50 mg, 0.158 mmol) in MeCN (5 mL) was added slowly POCl₃ (73 mg, 0.476 mmol) at 0° C., followed by DIPEA (62 mg, 0.476 mmol). After stirring at room temperature for 5 h, water (10 mL) was added and the mixture was extracted with EtOAc (3×20 mL). The combined organic layers were washed with brine (30 mL), dried over anhydrous Na₂SO₄, and concentrated in vacuo to give the crude, which was purified by preparative TLC (eluent: DCM/MeOH=20/1) to give t rac-(1S*,2S*)—N-(4-chloro-5-(methoxymethyl)pyrimidin-2-yl)-2-(4-methylpyrimidin-2-yl)cyclopropane-1-carboxamide (20 mg, 38%) as a white solid. ESI-MS [M+H]+: 334.1

Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide (I-621). To a solution of rac-(1S*,2S*)—N-(4-chloro-5-(methoxymethyl)pyrimidin-2-yl)-2-(4-methylpyrimidin-2-yl)cyclopropane-1-carboxamide (10 mg, 0.03 mmol) in i-PrOH (1 mL) were added 1-(2-(aminomethyl)-6-cyclopropylimidazo[1,2-a]pyridin-8-yl)-3-methylimidazolidine-2,4-dione (9 mg, 0.03 mmol) and DIPEA (12 mg, 0.09 mmol). After degassing with N₂ for 1 min, the reaction was irradiated in a microwave reactor at 140° C. for 2 h. The mixture was then concentrated in vacuo to get the crude, which was purified by preparative TLC (eluent: DCM/MeOH=10/1) to give the product rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide (10 mg. 56%) as a white solid. ESI-MS [M+H]+: 597.3. ¹H NMR (400 MHz, DMSO) δ 10.32 (s, 1H), 8.46 (d, J=5.1 Hz, 1H), 8.23 (s, 1H), 7.91 (s, 1H), 7.74 (s, 1H), 7.40-7.23 (m, 2H), 7.13 (d, J=5.0 Hz, 1H), 4.90 (s, 2H), 4.61 (d, J=5.3 Hz, 2H), 4.29 (s, 2H), 3.26 (s, 3H), 2.98 (s, 3H), 2.59-2.52 (m, 2H), 2.37 (s, 3H), 1.96-1.91 (m, 1H), 1.54-1.50 (m, 2H), 1.03-0.87 (m, 2H), 0.70-0.57 (m, 2H).

### Examples 622-623

### Synthesis of rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclo propane-1-carboxamide (I-622) and rac-(1S*,2S*)—N-(4-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methyl pyrimidin-2-yl)cyclopropane-1-carboxamide (I-623)

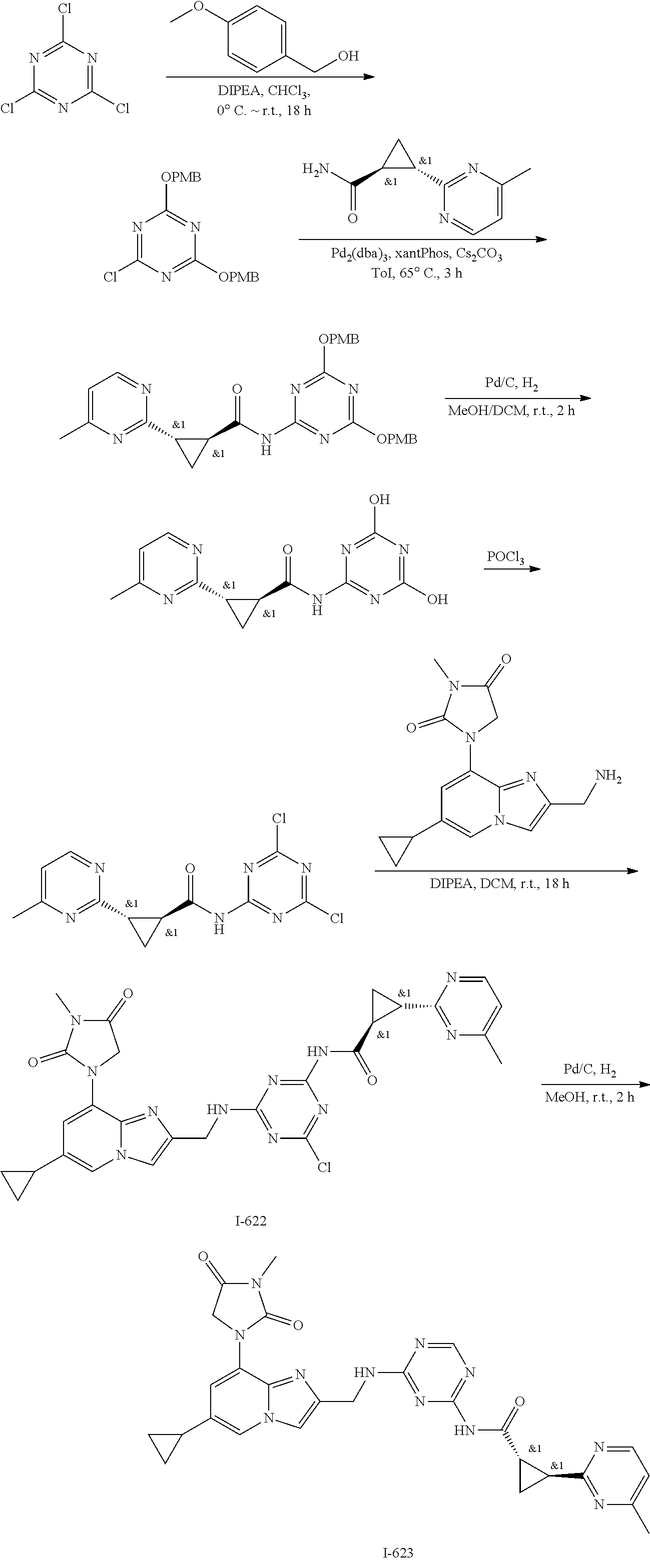

Synthesis of 2-chloro-4,6-bis((4-methoxybenzyl)oxy)-1,3,5-triazine. To a mixture of 2,4,6-trichloro-1,3,5-triazine (2 g, 10.9 mol) and (4-methoxyphenyl)methanol (3.0 g, 21.8 mol) in CHCl₃ (50 mL) was added DIPEA (4.2 g, 32.7 mol) at 0° C. After stirring at room temperature for 18 h, the reaction was quenched with saturated aq·NaHCO₃ (150 mL) and extracted with EtOAc (3×80 mL). The combined organic layers were washed with brine (80 mL), dried over Na₂SO₄ and concentrated in vacuo to get the crude, which was purified by column chromatography (eluent: EtOAc/PE=1/10) to give 2-chloro-4,6-bis((4-methoxybenzyl)oxy)-1,3,5-triazine (1.6 g, 38%) as a white solid. ESI-MS [M+H]+: 388.1.

Synthesis of rac-(1S*,2S*)—N-(4,6-bis((4-methoxybenzyl)oxy)-1,3,5-triazin-2-yl)-2-(4-methylpyrimidin-2-yl)cyclopropane-1-carboxamide. To a mixture of 2-chloro-4,6-bis((4-methoxybenzyl)oxy)-1,3,5-triazine (400 mg, 1.03 mmol), rac-(1S*,2S*)-2-(4-methylpyrimidin-2-yl)cyclopropane-1-carboxamide (182 mg, 1.03 mmol), and Cs₂CO₃ (674 mg, 2.07 mmol) in toluene (20 mL) were added Pd₂(dba)₃ (146 mg, 0.16 mmol) and XantPhos (179 mg, 0.31 mmol). The mixture was degassed with N₂ for three times and stirred at 65° C. in for 3 h. The mixture was diluted with H2O (100 mL) and extracted with EtOAc (3×50 mL). The combined organic layers were washed with brine (50 mL), dried over Na₂SO₄ and concentrated in vacuo. The residue was purified by silica gel column chromatography (eluent: MeOH/DCM=1/20) to give rac-(1S*,2S*)—N-(4,6-bis ((4-methoxybenzyl)oxy)-1,3,5-triazin-2-yl)-2-(4-methylpyrimidin-2-yl)cyclopropane-1-carboxamide (180 mg, 33%) as a white solid. ESI-MS [M+H]+: 529.1, ¹H NMR (400 MHz, DMSO) δ 11.16 (s, 1H), 8.49 (d, J=5.1 Hz, 1H), 7.44-7.33 (m, 4H), 7.16 (d, J=5.1 Hz, 1H), 6.97-6.89 (m, 4H), 5.34-5.21 (m, 4H), 3.03 (s, 1H), 2.62-2.55 (m, 1H), 2.36 (s, 3H), 1.65-1.55 (m, 2H).

Synthesis of rac-(1S*,2S*)—N-(4,6-dihydroxy-1,3,5-triazin-2-yl)-2-(4-methylpyrimidin-2-yl)cyclopropane-1-carboxamide. A mixture of rac-(1S*,2S*)—N-(4,6-bis((4-methoxybenzyl)oxy)-1,3,5-triazin-2-yl)-2-(4-methylpyrimidin-2-yl)cyclopropane-1-carboxamide (280 mg, 0.53 mmol) and Pd/C (100 mg) in MeOH/DCM (30 mL/30 mL) was degassed and stirred under H₂ at room temperature for 3 h. The mixture was filtered and concentrated in vacuo to give rac-(1S*,2S*)—N-(4,6-dihydroxy-1,3,5-triazin-2-yl)-2-(4-methylpyrimidin-2-yl)cyclopropane-1-carboxamide (140 mg, crude) as a white solid which was used in the next step directly. ESI-MS [M+H]+: 289.1

Synthesis of rac-(1S*,2S*)—N-(4,6-dichloro-1,3,5-triazin-2-yl)-2-(4-methylpyrimidin-2-yl)cyclopropane-1-carboxamide. A mixture of rac-(1S*,2S*)—N-(4,6-dihydroxy-1,3,5-triazin-2-yl)-2-(4-methylpyrimidin-2-yl)cyclopropane-1-carboxamide (140 mg, 0.48 mmol) and PhNEt₂ (107 mg, 0.72 mmol) in POCl₃ (5 mL) was degassed with N₂ and stirred at 120° C. for 3 h. The mixture was cooled to room temperature and concentrated in vacuo to give rac-(1S*,2S*)—N-(4,6-dichloro-1,3,5-triazin-2-yl)-2-(4-methylpyrimidin-2-yl)cyclopropane-1-carboxamide (250 mg, crude) as a brown oil which was used in the next step. ESI-MS [M+H]+: 325.1

Synthesis of rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-622). A mixture of rac-(1S*,2S*)—N-(4,6-dichloro-1,3,5-triazin-2-yl)-2-(4-methylpyrimidin-2-yl)cyclopropane-1-carboxamide (250 mg, 0.48 mmol), 1-(2-(aminomethyl)-6-cyclopropylimidazo[1,2-a]pyridin-8-yl)-3-methylimidazolidine-2,4-dione (144 mg, 0.48 mmol), and DIPEA (186 mg, 1.45 mmol) in DCM (10 mL) was stirred at room temperature for 18 h. The reaction was quenched with saturated aq·NaHCO₃ (50 mL) and extracted with EtOAc (3×50 mL). The combined organic layers were washed with brine (50 mL), dried over Na₂SO₄ and concentrated in vacuo. The residue was purified by preparative TLC (eluent: MeOH/DCM=1/15) to afford rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (75 mg, 27%) as a white solid. ESI-MS [M+H]+: 588.2. ¹H NMR (400 MHz, DMSO) δ 11.06-11.00 (m, 1H), 8.94-8.85 (m, 1H), 8.54-8.40 (m, 1H), 8.25-8.20 (m, 1H), 7.77-7.71 (m, 1H), 7.35-7.32 (m, 1H), 7.21-7.08 (m, 1H), 4.88 (s, 2H), 4.58-4.50 (m, 2H), 2.97 (s, 3H), 2.61-2.52 (m, 2H), 2.42-2.33 (s, 3H), 1.97-1.91 (m, 1H), 1.59-1.50 (m, 2H), 0.99-0.90 (m, 2H), 0.68-0.61 (m, 2H).

Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclo propane-1-carboxamide (I-623). A mixture of rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (45 mg, 0.077 mmol), and Pd/C (25 mg) in MeOH/DCM (5 mL/5 mL) was degassed and stirred under H₂ at RT for 2 h. The mixture was filtered, concentrated in vacuo to get the crude, & purified by preparative TLC (eluent: MeOH/DCM=1/15) to afford rac-(1S*,2S*)—N-(4-(((6-cyclopropyl-8-(3-methyl-2,4-dioxoimi-dazolidin-1-yl)imidazo[1,2-a]pyridin-2-yl)methyl)amino)-1,3,5-triazin-2-yl)-2-(4-methyl pyrimidin-2-yl)cyclopropane-1-carboxamide (20 mg, 48%) as a white solid. ESI-MS [M+H]+: 554.2. ¹H NMR (400 MHz, DMSO) δ 10.75-10.66 (m, 1H), 8.54-8.29 (m, 3H), 8.23 (s, 1H), 7.76-7.66 (m, 1H), 7.33 (s, 1H), 7.22-7.08 (m, 1H), 4.92-4.86 (m, 2H), 4.60-4.47 (m, 2H), 2.98-2.96 (m, 3H), 2.59-2.54 (m, 2H), 2.42-2.33 (m, 3H), 1.98-1.89 (m, 1H), 1.58-1.48 (m, 2H), 0.97-0.89 (m, 2H), 0.68-0.60 (m, 2H).

### Example 624

### Synthesis of rac-(1S*,2S*)—N-(7-(((6-cyclopropyl-8-(3-methyl-2,4-dioxoimidazolidin-1-yl)imidazo[1,2-a]pyridin-2-yl)methyl)amino)-[1,2,4]triazolo[1,5-a]pyrimidin-5-yl)-2-(4-methylpyrimidin-2-yl)cyclopropane-1-carboxamide (I-624)

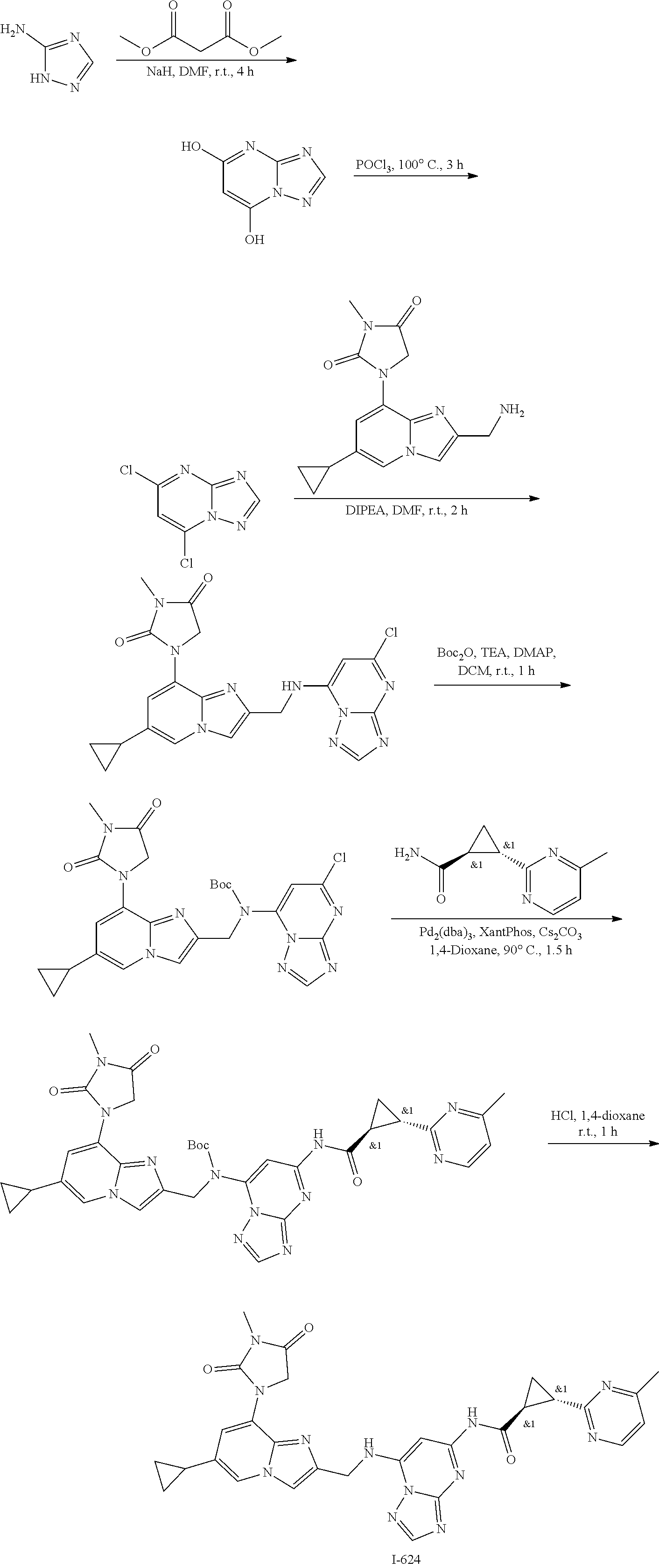

Synthesis of [1,2,4]triazolo[1,5-a]pyrimidine-5,7-diol. A mixture of [1,2,4]triazolo[1,5-a]pyrimidine-5,7-diol (5 g, 59.5 mmol), dimethyl malonate (11.5 g, 71.4 mmol), and NaH (2.8 g, 119 mmol, 600% dispersion in mineral oil) in DMF (25 mL) was stirred at RT for 4 h. The reaction was quenched with H2O (100 mL) and extracted with EtOAc (3×100 mL). The combined organic layers were washed with brine (60 mL), dried over Na₂SO₄, filtered and concentrated in vacuo to give the crude, purified by silica gel column chromatography (DCM/MeOH=10/1) to give [1,2,4]triazolo[1,5-a]pyrimidine-5,7-diol (2.6 g, 28%) as a white solid. ESI-MS [M+H]+: 153.2.

Synthesis of 5,7-dichloro-[1,2,4]triazolo[1,5-a]pyrimidine. A mixture of [1,2,4]triazolo[1,5-a]pyrimidine-5,7-diol (1.5 g, 9.9 mmol) and POCl₃ (15 mL) was stirred at 100° C. for 3 h. The reaction mixture was cooled to RT and concentrated in vacuo to give the crude, which was washed with saturated aq·NaHCO₃ (60 mL) and extracted with EtOAc (4×60 mL). The combined organic layers were washed with brine (60 mL), dried over Na₂SO₄ then concentrated in vacuo to give the crude. The residue was purified by silica gel column chromatography (PE/EtOAc=5/1) to give 5,7-dichloro-[1,2,4]triazolo[1,5-a]pyrimidine (400 mg, 22%) as a white solid. ESI-MS [M+H]+: 188.9.

Synthesis of 1-(2-(((5-chloro-[1,2,4]triazolo[1,5-a]pyrimidin-7-yl)amino)methyl)-6-cyclo propylimidazo[1,2-a]pyridin-8-yl)-3-methylimidazolidine-2,4-dione. A mixture of 5,7-dichloro-[1,2,4]triazolo[1,5-a]pyrimidine (400 mg, 2.1 mmol), 1-(2-(aminomethyl)-6-cyclopropylimidazo[1,2-a]pyridin-8-yl)-3-methylimidazolidine-2,4-dione hydrochloride (703 mg, 2.1 mmol), DIPEA (774 mg, 6 mmol), and DMF (20 mL) was stirred at room temperature for 2 h. The reaction was washed with water (30 mL) and extracted with EtOAc (3×50 mL). The combined organic layers were washed with brine (40 mL), dried over Na₂SO₄, filtered, and concentrated in vacuo to give the crude, which was purified by preparative TLC (DCM/MeOH=30/1) to give 1-(2-(((5-chloro-[1,2,4]triazolo[1,5-a]pyrimidin-7-yl)amino)methyl)-6-cyclopropylimidazo[1,2-a]pyridin-8-yl)-3-methylimidazolidine-2,4-dione (300 mg, 32%) as a white solid. ESI-MS [M+H]+: 452.2.

Synthesis of tert-butyl (5-chloro-[1,2,4]triazolo[1,5-a]pyrimidin-7-yl)((6-cyclopropyl-8-(3-methyl-2,4-dioxoimidazolidin-1-yl)imidazo[1,2-a]pyridin-2-yl)methyl)carbamate. A mixture of 1-(2-(((5-chloro-[1,2,4]triazolo[1,5-a]pyrimidin-7-yl)amino)methyl)-6-cyclopropylimidazo[1,2-a]pyridin-8-yl)-3-methylimidazolidine-2,4-dione (300 mg, 0.66 mmol), di-tert-butyl dicarbonate (216 mg, 0.99 mmol), TEA (200 mg, 1.98 mmol), and DMAP (80.5 mg, 0.66 mmol) in DCM (20 ml) was stirred at room temperature for 1 h. The reaction was washed with water (50 mL) and extracted with DCM (3×40 mL). The combined organic layers were washed with brine (40 mL), dried over Na₂SO₄, filtered, and concentrated in vacuo to give the crude, which was purified by silica gel column chromatography (DCM/MeOH=20/1) to give tert-butyl (5-chloro-[1,2,4]triazolo[1,5-a]pyrimidin-7-yl)((6-cyclopropyl-8-(3-methyl-2,4-dioxoimidazolidin-1-yl)imidazo[1,2-a]pyridin-2-yl)methyl)carbamate (300 mg, 82%) as a yellow solid. ESI-MS [M+H]+: 552.2.

Synthesis of rac-tert-butyl ((6-cyclopropyl-8-(3-methyl-2,4-dioxoimidazolidin-1-yl) imidazo[1,2-a]pyridin-2-yl)methyl)(5-((1S*,2S*)-2-(4-methylpyrimidin-2-yl)cyclopropane-1-carboxamido)-[1,2,4]triazolo[1,5-a]pyrimidin-7-yl)carbamate. To a mixture of tert-butyl (5-chloro-[1,2,4]triazolo[1,5-a]pyrimidin-7-yl)((6-cyclopropyl-8-(3-methyl-2,4-dioxoimidazolidin-1-yl) imidazo[1,2-a]pyridin-2-yl)methyl)carbamate (140 mg, 0.25 mmol), rac-(1S*,2S*)-2-(4-methyl pyrimidin-2-yl) cyclopropane-1-carboxamide (49 mg, 0.26 mmol), & Cs₂CO₃ (234.6 mg, 0.72 mmol) in 1,4-dioxane (5 mL) were added Pd₂(dba)₃ (33 mg, 0.038 mmol) and Xantphos (27.8 mg, 0.048 mmol). After stirring at 90° C. for 1.5 h, the reaction was washed with H2O (30 mL) and extracted with EtOAc (3×40 mL). The combined organic layers were washed with brine (30 mL), dried over Na₂SO₄, filtered, concentrated in vacuo to give the crude, & purified by preparative TLC (DCM/MeOH=20/1) to give rac-tert-butyl((6-cyclopropyl-8-(3-methyl-2,4-dioxoimidazolidin-1-yl)imidazo[1,2-a]pyridin-2-yl)methyl)(5-((1S*,2S*)-2-(4-methylpyrimidin-2-yl) cyclopropane-1-carboxamido)-[1,2,4]triazolo[1,5-a]pyrimidin-7-yl)carbamate (60 mg, 36%) as a yellow solid. ESI-MS [M+H]+: 693.2.

Synthesis of rac-(1S*,2S*)—N-(7-(((6-cyclopropyl-8-(3-methyl-2,4-dioxoimidazolidin-1-yl)imidazo[1,2-a]pyridin-2-yl)methyl)amino)-[1,2,4]triazolo[1,5-a]pyrimidin-5-yl)-2-(4-methyl pyrimidin-2-yl)cyclopropane-1-carboxamide (I-624). A mixture of rac-tert-butyl((6-cyclo propyl-8-(3-methyl-2,4-dioxoimidazolidin-1-yl)imidazo[1,2-a]pyridin-2-yl)methyl)(5-((1S*,2S*)-2-(4-methyl pyrimidin-2-yl)cyclopropane-1-carboxamido)-[1,2,4]triazolo[1,5-a]pyrimidin-7-yl) carbamate (60 mg, 0.086 mmol) in HCl (3 mL, 4M solution in 1,4-dioxane) was stirred at RT for 1 h. The reaction was concentrated in vacuo to give the crude, which was purified by preparative TLC (DCM/MeOH=20/1) to give rac-(1S*,2S*)—N-(7-(((6-cyclo propyl-8-(3-methyl-2,4-dioxoimidazolidin-1-yl)imidazo[1,2-a]pyridin-2-yl)methyl)amino)-[1,2,4]triazolo[1,5-a]pyrimidin-5-yl)-2-(4-methylpyrimidin-2-yl)cyclopropane-1-carboxamide (30 mg, 59%) as a white solid. ESI-MS [M+H]⁺: 593.2. ¹H NMR (400 MHz, DMSO) δ 11.04 (s, 1H), 8.80 (t, J=6.1 Hz, 1H), 8.52 (d, J=5.1 Hz, 1H), 8.34 (s, 1H), 8.23 (s, 1H), 7.80 (s, 1H), 7.46 (d, J=21.5 Hz, 1H), 7.38 (s, 1H), 7.20 (d, J=5.1 Hz, 1H), 4.98 (d, J=4.4 Hz, 2H), 4.65 (d, J=5.9 Hz, 2H), 2.99 (s, 3H), 2.69-2.54 (m, 2H), 2.41 (s, 3H), 1.98-1.90 (m, 1H), 1.55-1.52 (m, 2H), 0.99-0.85 (m, 2H), 0.70-0.58 (m, 2H).

### Example 625

### Synthesis of rac-(1S*,2S*)—N-(6-(2-(6-cyclopropyl-8-(3-methyl-2,4-dioxoimidazolidin-1-yl) imidazo[1,2-a]pyridin-2-yl)ethyl)pyrimidin-4-yl)-2-(4-methylpyrimidin-2-yl)cyclopropane-1-carboxamide (I-625)

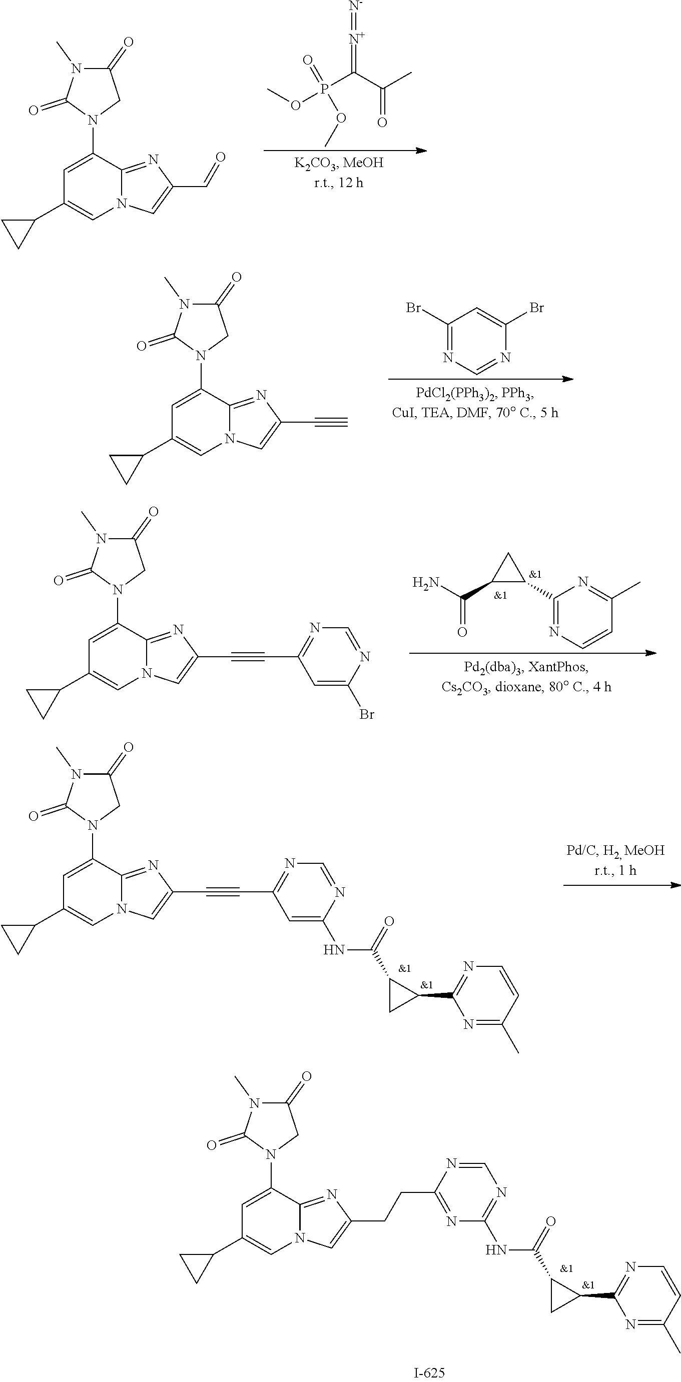

Synthesis of 1-(6-cyclopropyl-2-ethynylimidazo[1,2-a]pyridin-8-yl)-3-methylimidazolidine-2,4-dione. To a solution of 6-cyclopropyl-8-(3-methyl-2,4-dioxoimidazolidin-1-yl)imidazo[1,2-a]pyridine-2-carbaldehyde (700 mg, 2.35 mmol) in MeOH (30 mL) were added dimethyl (1-diazo-2-oxopropyl)phosphonate (2.25 g, 11.75 mmol) and K₂CO₃ (1.62 g, 11.75 mmol). The reaction mixture was stirred at room temperature for 12 h. The reaction was diluted with water (60 mL) and extracted with EtOAc (3×50 mL). The combined organic layers were washed with brine (40 mL), dried over Na₂SO₄, filtered and concentrated in vacuo to give the crude, which was purified by silica gel column chromatography (eluent: DCM/MeOH=30/1) to give 1-(6-cyclopropyl-2-ethynylimidazo[1,2-a]pyridin-8-yl)-3-methylimidazolidine-2,4-dione (250 mg, 36%) as a light yellow oil. ESI-MS [M+H]+: 295.2

Synthesis of 1-(2-((6-bromopyrimidin-4-yl)ethynyl)-6-cyclopropylimidazo[1,2-a]pyridin-8-yl)-3-methylimidazolidine-2,4-dione. To a solution of 1-(6-cyclopropyl-2-ethynylimidazo[1,2-a]pyridin-8-yl)-3-methylimidazolidine-2,4-dione (250 mg, 0.85 mmol), 4,6-dibromopyrimidine (300 mg, 1.28 mmol) PdCl₂(PPh₃)₂ (60 mg, 0.085 mmol), CuI (16 mg, 0.085 mmol), and TEA (1 mL) in DMF (20 mL) was stirred at 70° C. for 5 h under N₂. After cooling to room temperature, the mixture washed with water (50 mL) and extracted with EtOAc (3×30 mL). The combined organic layers were washed with brine (40 mL), dried over Na₂SO₄, filtered, and concentrated in vacuo to give the crude, which was purified by silica gel column chromatography (eluent: DCM/MeOH=20/1) to give the 1-(2-((6-bromopyrimidin-4-yl)ethynyl)-6-cyclopropylimidazo[1,2-a]pyridin-8-yl)-3-methylimidazolidine-2,4-dione (100 mg, 17%) as a yellow solid. ESI-MS [M+H]+: 451.2.

Synthesis of rac-(1S*,2S*)—N-(6-((6-cyclopropyl-8-(3-methyl-2,4-dioxoimidazolidin-1-yl)imidazo[1,2-a]pyridin-2-yl)ethynyl)pyrimidin-4-yl)-2-(4-methylpyrimidin-2-yl)cyclopropane-1-carboxamide. A mixture of 1-(2-((6-bromopyrimidin-4-yl)ethynyl)-6-cyclopropylimidazo[1,2-a]pyridin-8-yl)-3-methylimidazolidine-2,4-dione (100 mg, 0.22 mmol), rac-(1S*,2S*)-2-(4-methylpyrimidin-2-yl)cyclopropane-1-carboxamide (58 mg, 0.33 mmol), Pd₂(dba)₃ (20 mg, 0.022 mmol), Xantphos (13 mg, 0.022 mmol), and Cs₂CO₃ (215 mg, 0.66 mmol) in 1,4-dioxane (10 mL) was stirred at 80° C. for 4 h under nitrogen. The reaction was washed with water (30 mL) and extracted with EtOAc (3×30 mL). The combined organic layers were washed with brine (30 mL), dried over Na₂SO₄ then concentrated in vacuo to give the crude, which was purified by silica gel column chromatography (eluent: DCM/MeOH=20/1) to give the rac-(1S*,2S*)—N-(6-((6-cyclo propyl-8-(3-methyl-2,4-dioxoimidazolidin-1-yl)imidazo[1,2-a]pyridin-2-yl)ethynyl)pyrimidin-4-yl)-2-(4-methylpyrimidin-2-yl)cyclopropane-1-carboxamide (40 mg, 33%) as a light yellow solid. ESI-MS [M+H]+: 548.2.

Synthesis of rac-(1S*,2S*)—N-(6-(2-(6-cyclopropyl-8-(3-methyl-2,4-dioxoimidazolidin-1-yl)imidazo[1,2-a]pyridin-2-yl)ethyl)pyrimidin-4-yl)-2-(4-methylpyrimidin-2-yl)cyclopropane-1-carboxamide (I-625). To a solution of rac-(1S*,2S*)—N-(6-((6-cyclopropyl-8-(3-methyl-2,4-dioxoimidazolidin-1-yl)imidazo[1,2-a]pyridin-2-yl)ethynyl)pyrimidin-4-yl)-2-(4-methylpyrimidin-2-yl)cyclopropane-1-carboxamide (40 mg, 0.073 mmol) and Pd/C (10 mg) in MeOH (15 mL) was stirred at room temperature for 1 h under H₂. The reaction was filtered and concentrated in vacuo to give the crude, which was purified by preparative TLC (DCM/MeOH=15/1) to give rac-(1S*,2S*)—N-(6-(2-(6-cyclopropyl-8-(3-methyl-2,4-dioxoimidazolidin-1-yl)imidazo[1,2-a]pyridin-2-yl)ethyl)pyrimidin-4-yl)-2-(4-methylpyrimidin-2-yl)cyclopropane-1-carboxamide (10 mg, 25%) as a white solid. ESI-MS [M+H]+: 555.2. ¹H NMR (400 MHz, MeOD) δ 8.68 (s, 1H), 8.47 (d, J=5.1 Hz, 1H), 8.12 (s, 1H), 7.95 (s, 1H), 7.53 (s, 1H), 7.25 (s, 1H), 7.17 (d, J=5.1 Hz, 1H), 4.64 (s, 2H), 3.16 (s, 4H), 3.10 (s, 3H), 2.68-2.62 (m, 1H), 2.56-2.50 (m, 1H), 2.47 (s, 3H), 1.98-1.92 (m, 1H), 1.70-1.56 (m, 2H), 1.00-0.93 (m, 2H), 0.75-0.71 (m, 2H).

### Example 626

### Synthesis of rac-(1S*,2S*)—N-(3-((6-cyclopropyl-8-(3-methyl-2,4-dioxoimidazolidin-1-yl)imidazo[1,2-a]pyridin-2-yl)methyl)-2-oxo-2,3-dihydrooxazolo[4,5-d]pyrimidin-5-yl)-2-(4-methylpyrimidin-2-yl)cyclopropane-1-carboxamide (I-626)

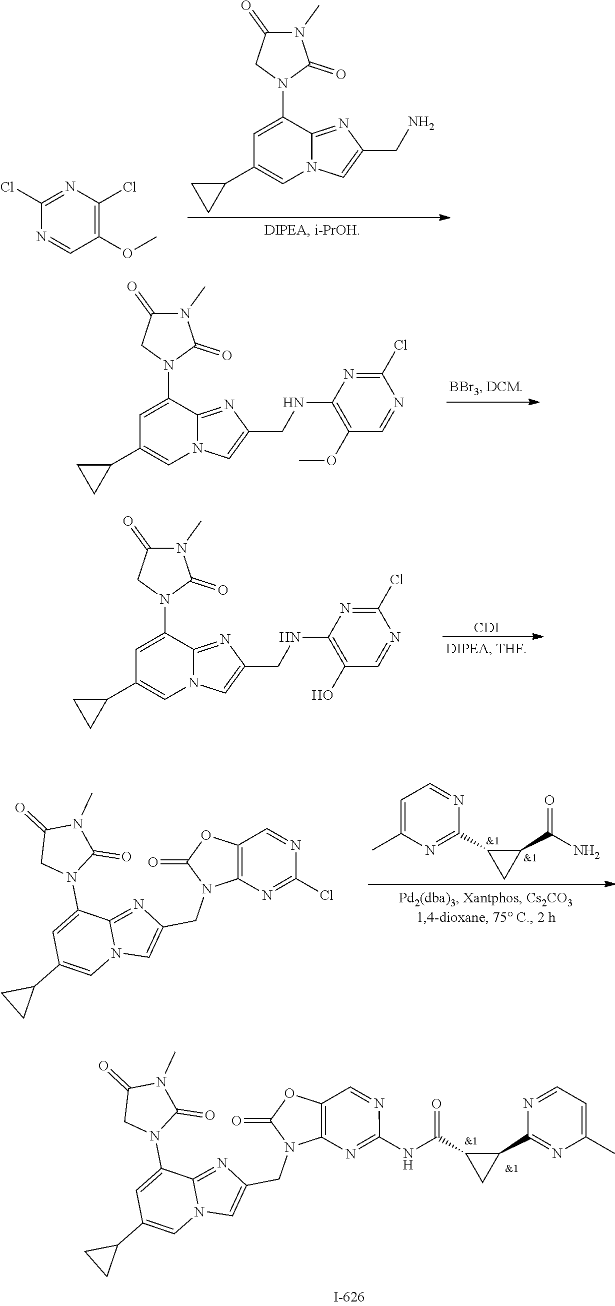

Synthesis of 1-(2-(((2-chloro-5-methoxypyrimidin-4-yl)amino)methyl)-6-cyclopropylimidazo[1,2-a]pyridin-8-yl)-3-methylimidazolidine-2,4-dione. A mixture of 2,4-dichloro-5-methoxypyrimidine (300 mg, 1.0 mmol), 1-(2-(aminomethyl)-6-cyclopropylimidazo[1,2-a]pyridin-8-yl)-3-methylimidazolidine-2,4-dione (356 mg, 2.0 mmol), and DIPEA (645 mg, 5.0 mmol) in i-PrOH (10 mL) was stirred at 60° C. for 16 h under N₂. The mixture was concentrated in vacuo to give the crude, which was purified by Prep-TLC (eluent: DCM/MeOH=10/1) to give 1-(2-(((2-chloro-5-methoxypyrimidin-4-yl)amino)methyl)-6-cyclopropylimidazo[1,2-a]pyridin-8-yl)-3-methylimidazolidine-2,4-dione as a white solid (260 mg, 59%). ESI-MS [M+H]⁺: 442.0.

Synthesis of 1-(2-(((2-chloro-5-hydroxypyrimidin-4-yl)amino)methyl)-6-cyclopropylimidazo[1,2-a]pyridin-8-yl)-3-methylimidazolidine-2,4-dione. To a mixture of 1-(2-(((2-chloro-5-methoxypyrimidin-4-yl)amino)methyl)-6-cyclopropylimidazo[1,2-a]pyridin-8-yl)-3-methylimidazolidine-2,4-dione (260 mg, 0.59 mmol) in DCM (10 mL) was added BBr₃ (731 mg, 2.95 mmol) at 0° C. under N₂. After stirring for 36 h at room temperature under N₂, the reaction was quenched with saturated aq·NaHCO₃ (15 mL) and extracted with EtOAc (3×10 mL). The combined organic layers were washed with brine (20 mL), dried over anhydrous Na₂SO₄, and concentrated in vacuo to give the crude product, which was purified by Prep-TLC (eluent: DCM/MeOH=10/1) to give 1-(2-(((2-chloro-5-hydroxypyrimidin-4-yl)amino)methyl)-6-cyclopropylimidazo[1,2-a]pyridin-8-yl)-3-methylimidazolidine-2,4-dione (110 mg, 44%). as a white solid. ESI-MS [M+H]⁺: 428.1.

Synthesis of 1-(2-((5-chloro-2-oxooxazolo[4,5-d]pyrimidin-3(2H)-yl)methyl)-6-cyclopropylimidazo[1,2-a]pyridin-8-yl)-3-methylimidazolidine-2,4-dione. A mixture of 1-(2-(((2-chloro-5-hydroxypyrimidin-4-yl)amino)methyl)-6-cyclopropylimidazo[1,2-a]pyridin-8-yl)-3-methylimidazolidine-2,4-dione (80 mg, 0.19 mmol) and CDI (152 mg, 0.94 mmol) in THF (5 mL) was stirred for 2 h at 60° C. under N₂. The mixture was concentrated in vacuo to give the crude product, which was purified by Prep-TLC (eluent: DCM/MeOH=20/1) to give 1-(2-((5-chloro-2-oxooxazolo[4,5-d]pyrimidin-3(2H)-yl)methyl)-6-cyclopropylimidazo[1,2-a]pyridin-8-yl)-3-methylimidazolidine-2,4-dione (70 mg, 81%) as a white solid. ESI-MS [M+H]⁺: 454.1.

Synthesis of rac-(1S*,2S*)—N-(3-((6-cyclopropyl-8-(3-methyl-2,4-dioxoimidazolidin-1-yl)imidazo[1,2-a]pyridin-2-yl)methyl)-2-oxo-2,3-dihydrooxazolo[4,5-d]pyrimidin-5-yl)-2-(4-methylpyrimidin-2-yl)cyclopropane-1-carboxamide (I-626). A mixture of 1-(2-((5-chloro-2-oxooxazolo[4,5-d]pyrimidin-3(2H)-yl)methyl)-6-cyclopropylimidazo[1,2-a]pyridin-8-yl)-3-methylimidazolidine-2,4-dione (20 mg, 0.044 mmol), rac-(1S*,2S*)-2-(4-methylpyrimidin-2-yl)cyclopropane-1-carboxamide (7.8 mg, 0.044 mmol), Pd₂(dba)₃ (4 mg, 0.0044 mmol), Xantphos (3 mg, 0.0044 mmol), and Cs₂CO₃ (43 mg, 0.13 mmol) in 1,4-dioxane (3 mL) was stirred for 2 h at 75° C. under N₂. The reaction mixture was filtered through Celite® and the filter cake was washed with DCM/MeOH (10/1, 10 mL). The filtrate was concentrated in vacuo to give the crude, which was purified by preparative HPLC to give rac-(1S*,2S*)—N-(3-((6-cyclopropyl-8-(3-methyl-2,4-dioxoimidazolidin-1-yl)imidazo[1,2-a]pyridin-2-yl)methyl)-2-oxo-2,3-dihydrooxazolo[4,5-d]pyrimidin-5-yl)-2-(4-methylpyrimidin-2-yl)cyclopropane-1-carboxamide (4 mg, 15%) as a white solid. ESI-MS [M+H]+: 595.2. ¹H NMR (400 MHz, DMSO) δ 10.99 (s, 1H), 8.49-8.47 (m, 2H), 8.23-8.19 (m, 1H), 7.94 (s, 1H), 7.37 (d, J=1.3 Hz, 1H), 7.16 (d, J=5.1 Hz, 1H), 5.02 (s, 2H), 4.80 (s, 2H), 2.95 (s, 3H), 2.76-2.67 (m, 2H), 2.40 (s, 3H), 1.99-1.92 (m, 1H), 1.54-1.50 (m, 2H), 0.96-0.85 (m, 2H), 0.70-0.62 (m, 2H).

### Example 627

### Synthesis of rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(hydroxymethyl)pyrimidin-2-yl)-2-(4-methylpyrimidin-2-yl)cyclopropane-1-carboxamide (I-627)

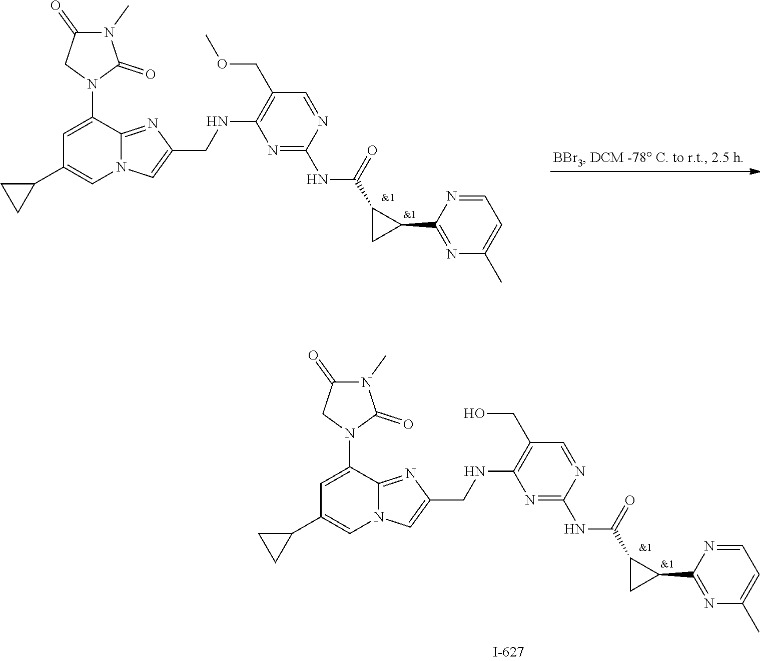 [SMILES: COCc1cnc(NC(=O)[C@H]2C[C@@H]2c2nccc(C)n2)nc1NCc1cn2cc(C3CC3)cc(N3CC(=O)N(C)C3=O)c2n1 ; Cc1ccnc([C@H]2C[C@@H]2C(=O)Nc2ncc(CO)c(NCc3cn4cc(C5CC5)cc(N5CC(=O)N(C)C5=O)c4n3)n2)n1]

To a solution of rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide (100 mg, 0.167 mmol) in DCM (8 mL) was added BBr₃ (251 mg, 1.0 mmol) at −78° C. The reaction was allowed warm to room temperature and it was stirred at room temperature for 2.5 h. Then the mixture was concentrated in vacuo to get the crude, which was purified by preparative HPLC to give rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(hydroxymethyl)pyrimidin-2-yl)-2-(4-methylpyrimidin-2-yl)cyclopropane-1-carboxamide (15 mg, 15%) as a white solid. ESI-MS [M+H]+: 583.2. ¹H NMR (400 MHz, DMSO) δ 10.26 (s, 1H), 8.46 (d, J=5.1 Hz, 1H), 8.38 (s, 1H), 8.22 (d, J=1.0 Hz, 1H), 7.88 (s, 1H), 7.76 (s, 1H), 7.33 (d, J=1.4 Hz, 1H), 7.22-7.27 (m, 1H), 7.13 (d, J=5.1 Hz, 1H), 5.16 (s, 1H), 4.89 (s, 2H), 4.61 (d, J=5.4 Hz, 2H), 4.35 (s, 2H), 2.98 (s, 3H), 2.52-2.56 (m, 2H), 2.37 (s, 3H), 1.97-1.91 (m, 1H), 1.53-1.50 (m, 2H), 1.02-0.87 (m, 2H), 0.71-0.55 (m, 2H).

### Example 628

### Synthesis of rac-(1S*,2S*)—N-(4-((6-cyclopropyl-8-(3-methyl-2,4-dioxoimidazolidin-1-yl)imidazo[1,2-a]pyridin-2-yl)methoxy)-5-(trifluoromethyl)pyrimidin-2-yl)-2-(4-methylpyrimidin-2-yl)cyclopropane-1-carboxamide (I-628)

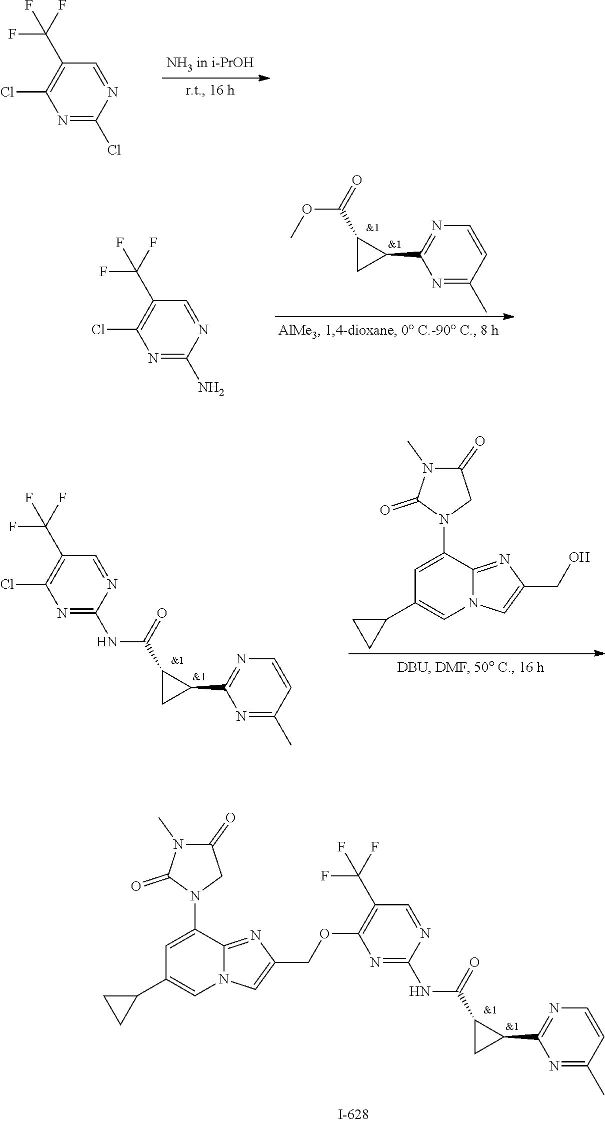

Synthesis of 4-chloro-5-(trifluoromethyl)pyrimidin-2-amine. A mixture of 2,4-dichloro-5-(trifluoromethyl)pyrimidine (1 g, 4.6 mmol) in ammonia solution (2M in i-PrOH, 40 mL) was stirred at room temperature for 16 h. The reaction mixture was concentrated in vacuo to give the crude, which was purified by silica gel column chromatography (eluent: EtOAc/PE=1/8) to give 4-chloro-5-(trifluoromethyl)pyrimidin-2-amine (350 mg, 39%) as a white solid. ESI-MS [M+H]⁺: 198.0.

Synthesis of rac-(1S*,2S*)—N-(4-chloro-5-(trifluoromethyl)pyrimidin-2-yl)-2-(4-methylpyrimidin-2-yl)cyclopropane-1-carboxamide. To a solution of 4-chloro-5-(trifluoromethyl)pyrimidin-2-amine (350 mg, 1.78 mmol) in 1,4-dioxane (10 mL) was added AlMe₃ (2M in 1,4-dioxane, 1.2 mL, 2.4 mmol) at 0° C. under N₂ and stirred at 30° C. for 2 h. To this resulting mixture was added a solution of rac-methyl (1S*,2S*)-2-(4-methylpyrimidin-2-yl)cyclopropane-1-carboxylate (386 mg, 1.78 mmol) in 1,4-dioxane (10 mL). After stirring at 90° C. for 8 h under N₂, the reaction was quenched with water (30 mL) and extracted with EtOAc (3×30 mL). The combined organic layers were washed with brine (20 mL), dried over anhydrous Na₂SO₄, and concentrated in vacuo to give the crude product, which was purified by silica gel column chromatography (eluent: EtOAc/PE=1/1) to give rac-(1S*,2S*)—N-(4-chloro-5-(trifluoromethyl)pyrimidin-2-yl)-2-(4-methylpyrimidin-2-yl)cyclopropane-1-carboxamide (270 mg, 43%) as a white solid. ESI-MS [M+H]⁺: 358.1.

Synthesis of rac-(1S*,2S*)—N-(4-((6-cyclopropyl-8-(3-methyl-2,4-dioxoimidazolidin-1-yl)imidazo[1,2-a]pyridin-2-yl)methoxy)-5-(trifluoromethyl)pyrimidin-2-yl)-2-(4-methylpyrimidin-2-yl)cyclopropane-1-carboxamide (I-628). A mixture of rac-(1S*,2S*)—N-(4-chloro-5-(trifluoromethyl)pyrimidin-2-yl)-2-(4-methylpyrimidin-2-yl)cyclopropane-1-carboxamide (60 mg, 0.168 mmol), 1-(6-cyclopropyl-2-(hydroxymethyl)imidazo[1,2-a]pyridin-8-yl)-3-methylimidazolidine-2,4-dione (50 mg, 0.168 mmol), and DBU (25.5 mg, 0.168 mmol) in DMF (5 mL) was stirred at 50° C. for 16 h. The reaction was diluted with water (20 mL) and extracted with EtOAc (3×20 mL). The organic layer was concentrated in vacuo and purified by preparative HPLC to give rac-(1S*,2S*)—N-(4-((6-cyclopropyl-8-(3-methyl-2,4-dioxoimidazolidin-1-yl)imidazo[1,2-a]pyridin-2-yl)methoxy)-5-(trifluoromethyl)pyrimidin-2-yl)-2-(4-methylpyrimidin-2-yl)cyclopropane-1-carboxamide (10 mg, 10%) as a white solid. ESI-MS [M+H]+: 622.2. ¹H NMR (400 MHz, DMSO) δ 11.34 (s, 1H), 8.74 (s, 1H), 8.50 (d, J=5.1 Hz, 1H), 8.30 (s, 1H), 8.00 (s, 1H), 7.40 (s, 1H), 7.17 (d, J=5.1 Hz, 1H), 5.60 (s, 2H), 4.87 (s, 2H), 2.97 (s, 3H), 2.68-2.58 (m, 1H), 2.54 (s, 1H), 2.39 (s, 3H), 2.05-1.88 (m, 1H), 1.63-1.60 (m, 2H), 1.02-0.90 (m, 2H), 0.69-0.65 (m, 2H).

### Example 629

### Synthesis of (1S,2S)—N-(2-((6-cyclopropyl-8-(3-methyl-2,4-dioxoimidazolidin-1-yl)imidazo[1,2-a]pyridin-2-yl)methyl)oxazolo[5,4-c]pyridin-4-yl)-2-(4-methylpyrimidin-2-yl)cyclopropane-1-carboxamide (I-629)

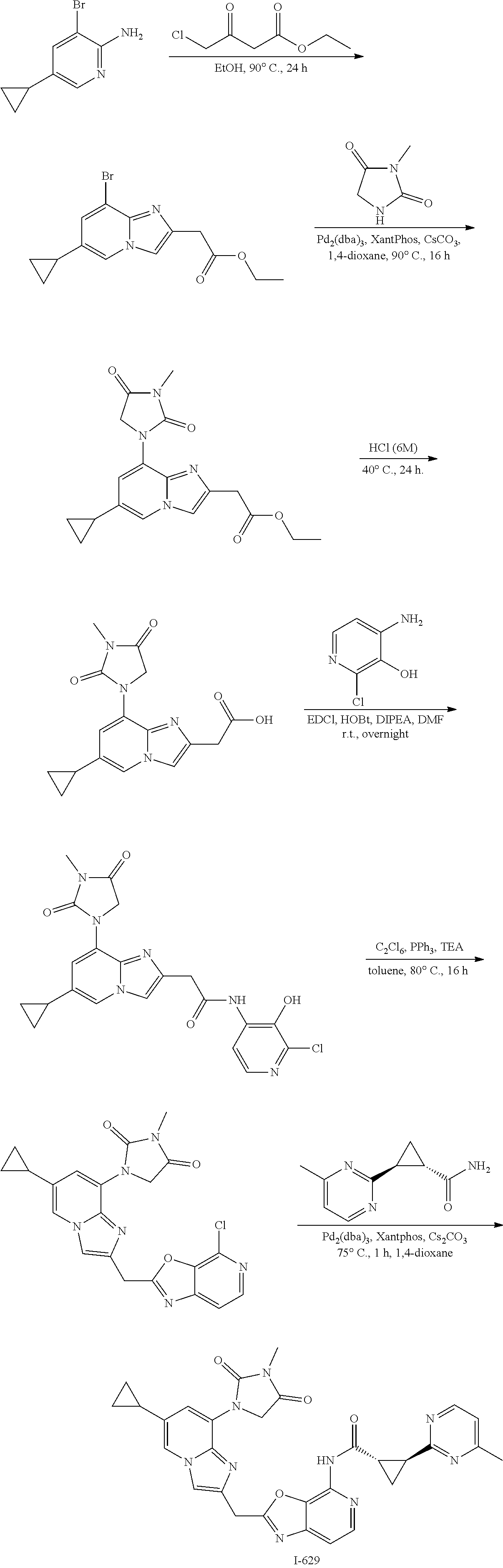

Synthesis of ethyl 2-(8-bromo-6-cyclopropylimidazo[1,2-a]pyridin-2-yl)acetate. A mixture of ethyl 4-chloro-3-oxobutanoate (3.0 g, 18 mmol) and 3-bromo-5-cyclopropylpyridin-2-amine (2.5 g, 12 mmol) in EtOH (50 mL) was stirred at 90° C. for 24 h. The reaction mixture was concentrated in vacuo to give the crude, which was purified by column chromatography (eluent: PE/EtOAc=5/1) to give ethyl 2-(8-bromo-6-cyclopropylimidazo[1,2-a]pyridin-2-yl)acetate (3.0 g, 78%) as a white solid. ESI-MS [M+H]+: 323.2.

Synthesis of ethyl 2-(6-cyclopropyl-8-(3-methyl-2,4-dioxoimidazolidin-1-yl)imidazo[1,2-a]pyridin-2-yl)acetate. To a solution of ethyl 2-(8-bromo-6-cyclopropylimidazo[1,2-a]pyridin-2-yl)acetate (3.0 g, 9.3 mmol), 3-methylimidazolidine-2,4-dione (3.2 g, 27.9 mmol), and Cs₂CO₃ (9.1 g, 27.9 mmol) in 1,4-dioxane (50 mL) was added Pd₂(dba)₃ (431 mg, 0.47 mmol) and Xantphos (538 mg, 0.93 mmol). The reaction mixture was stirred at 90° C. for 16 h under N₂. The reaction mixture was filtered through Celite® and the filter cake was washed with DCM/MeOH (10/1, 100 mL). The filtrate was concentrated in vacuo to give the crude, which was purified by column chromatography (eluent: DCM/MeOH=40/1) to give ethyl 2-(6-cyclopropyl-8-(3-methyl-2,4-dioxoimidazolidin-1-yl)imidazo[1,2-a]pyridin-2-yl)acetate (2.8 g, 85%) as a yellow solid. ESI-MS [M+H]+: 357.2.

Synthesis of 2-(6-cyclopropyl-8-(3-methyl-2,4-dioxoimidazolidin-1-yl)imidazo[1,2-a]pyridin-2-yl)acetic acid. A solution of ethyl 2-(6-cyclopropyl-8-(3-methyl-2,4-dioxoimidazolidin-1-yl)imidazo[1,2-a]pyridin-2-yl)acetate (2.8 g, 7.9 mmol) in HCl (6M solution in water, 50 mL) was stirred at 40° C. for 24 h. The reaction mixture was concentrated in vacuo to give 2-(6-cyclopropyl-8-(3-methyl-2,4-dioxoimidazolidin-1-yl)imidazo[1,2-a]pyridin-2-yl)acetic acid (2.4 g, 93%) as a yellow solid, which was used in the next step without further purification. ESI-MS [M+H]+: 329.2.

Synthesis of N-(2-chloro-3-hydroxypyridin-4-yl)-2-(6-cyclopropyl-8-(3-methyl-2,4-dioxoimidazolidin-1-yl)imidazo[1,2-a]pyridin-2-yl)acetamide. A mixture of 2-(6-cyclopropyl-8-(3-methyl-2,4-dioxoimidazolidin-1-yl)imidazo[1,2-a]pyridin-2-yl) acetic acid (0.40 g, 1.2 mmol), 4-amino-2-chloropyridin-3-ol (0.18 g, 1.3 mmol), EDCI (0.35 g, 1.8 mmol), HOBt (0.25 g, 1.9 mmol), and DIPEA (0.47 g, 3.6 mmol) in dry DMF (10 mL) was stirred at room temperature for 16 h. The reaction was quenched with water (50 mL) and extracted with EtOAc (3×30 mL). The combined organic layers were washed with brine (sat·aq·, 20 mL), dried over anhydrous Na₂SO₄, and concentrated in vacuo to give the crude product, which was purified by preparative TLC (eluent: DCM/MeOH=15/1) to give N-(2-chloro-3-hydroxypyridin-4-yl)-2-(6-cyclopropyl-8-(3-methyl-2,4-dioxoimidazolidin-1-yl)imidazo[1,2-a]pyridin-2-yl)acetamide (0.22 g, 40%) as a light-yellow solid. ESI-MS [M+H]⁺: 455.1.

Synthesis of 1-(2-((4-chlorooxazolo[5,4-c]pyridin-2-yl)methyl)-6-cyclopropylimidazo[1,2-a]pyridin-8-yl)-3-methylimidazolidine-2,4-dione. A mixture of N-(2-chloro-3-hydroxypyridin-4-yl)-2-(6-cyclopropyl-8-(3-methyl-2,4-dioxoimidazolidin-1-yl)imidazo[1,2-a]pyridin-2-yl)acetamide (0.22 g, 0.48 mmol), perchloroethane (0.34 g, 1.4 mmol), TEA (0.51 mL, 3.7 mmol), and PPh₃ (0.30 g, 1.1 mmol) in toluene (10 mL) was stirred at 80° C. for 16 h under N₂. The reaction mixture was filtered through Celite® and the filter cake was washed with DCM/MeOH (10/1, 30 mL). The filtrate was concentrated in vacuo to give the crude, which was purified by preparative HPLC to give 1-(2-((4-chlorooxazolo[5,4-c]pyridin-2-yl)methyl)-6-cyclopropylimidazo[1,2-a]pyridin-8-yl)-3-methylimidazolidine-2,4-dione (0.11 g, 52%) as a white solid. ESI-MS [M+H]⁺: 437.2.

Synthesis of (1S,2S)—N-(2-((6-cyclopropyl-8-(3-methyl-2,4-dioxoimidazolidin-1-yl)imidazo[1,2-a]pyridin-2-yl)methyl)oxazolo[5,4-c]pyridin-4-yl)-2-(4-methylpyrimidin-2-yl)cyclopropane-1-carboxamide (I-629). To a mixture of 1-(2-((4-chlorooxazolo[5,4-c]pyridin-2-yl)methyl)-6-cyclopropylimidazo-[1,2-a]pyridin-8-yl)-3-methylimidazolidine-2,4-dione (44 mg, 0.10 mmol), (1S,2S)-2-(4-methylpyrimidin-2-yl) cyclopropane-1-carboxamide (18 mg, 0.10 mmol), and Cs₂CO₃ (98 mg, 0.30 mmol) in dioxane (6.0 mL) were added Pd₂(dba)₃ (9.0 mg, 0.0098 mmol) and Xant-phos (12 mg, 0.021 mmol). After stirring at 75° C. for 1 h under N₂, the mixture was filtered through Celite® and the filter cake was washed with DCM/MeOH (10/1, 20 mL). The filtrate was concentrated in vacuo to give the crude, which was purified by preparative HPLC to give (1S,2S)—N-(2-((6-cyclopropyl-8-(3-methyl-2,4-dioxoimidazolidin-1-yl)imidazo[1,2-a]pyridin-2-yl)methyl)oxazolo[5,4-c]pyridin-4-yl)-2-(4-methylpyrimidin-2-yl)cyclopropane-1-carboxamide (2.0 mg, 3%) as a yellow solid. ESI-MS [M+H]+: 578.2. ¹H NMR (400 MHz, DMSO) δ 11.04 (s, 1H), 8.54 (d, J=5.1 Hz, 1H), 8.40-8.10 (m, 2H), 7.92 (s, 1H), 7.60 (d, J=5.4 Hz, 1H), 7.37 (s, 1H), 7.23 (d, J=5.1 Hz, 1H), 4.85 (s, 2H), 4.52 (s, 2H), 2.98 (s, 3H), 2.62-2.55 (m, 2H), 2.43 (s, 3H), 1.99-1.93 (m, 1H), 1.60-1.55 (m, 2H), 0.99-0.91 (m, 2H), 0.71-0.62 (m, 2H).

### Example 630

### Synthesis of (1S,2S)—N-(3-((6-cyclopropyl-8-(3-methyl-2,4-dioxoimidazolidin-1-yl)imidazo[1,2-a]pyridin-2-yl)methyl)-[1,2,4]triazolo[4,3-a]pyrazin-6-yl)-2-(4-methylpyrimidin-2-yl)cyclopropane-1-carboxamide (I-630)

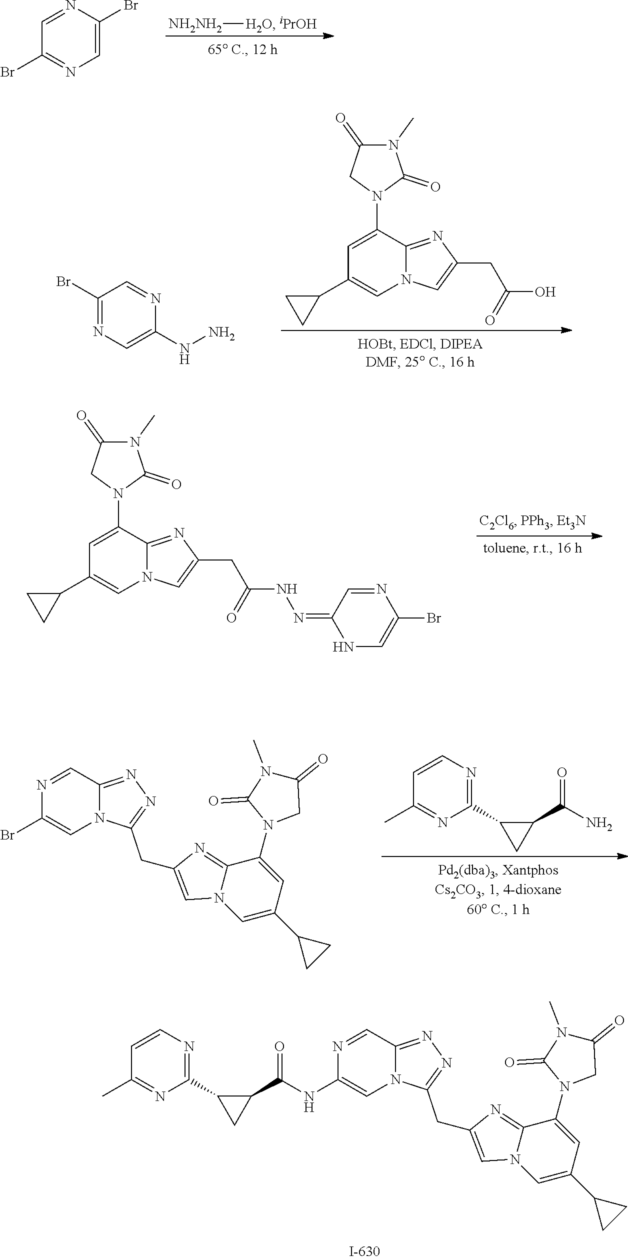

Synthesis of 2-bromo-5-hydrazineylpyrazine. A mixture of 2,5-dibromopyrazine (1.0 g, 4.2 mmol) and NH₂NH₂—H₂O (4 g, purity: 80%, 64 mmol) in ⁱPrOH (20 mL) was stirred at 65° C. for 12 h. The reaction mixture was poured into water (50 mL) and extracted with EtOAc (3×50 mL). The combined organic layers were washed with brine (30 mL), dried over anhydrous Na₂SO₄, and concentrated in vacuo to give the crude, which was purified by column chromatography on silica gel (eluent: PE/EtOAc=5/1) to give 2-bromo-5-hydrazineylpyrazine (0.70 g, 88%) as a yellow oil. ESI-MS [M+H]+: 188.1

Synthesis of (E)-N′-(5-bromopyrazin-2(1H)-ylidene)-2-(6-cyclopropyl-8-(3-methyl-2,4-dioxoimidazolidin-1-yl)imidazo[1,2-a]pyridin-2-yl)acetohydrazide. A mixture of 2-bromo-5-hydrazineylpyrazine (0.20 g, 1.1 mmol), 2-(6-cyclopropyl-8-(3-methyl-2,4-dioxoimidazolidin-1-yl)imidazo[1,2-a]pyridin-2-yl)acetic acid (0.35 g, 1.1 mmol), EDCI (0.40 g, 2.1 mmol), HOBt (0.29 g, 2.1 mmol), and DIPEA (0.19 g, 1.5 mmol) in dioxane (8.0 mL) was stirred at 25° C. for 16 h. The mixture was diluted with EtOAc (3×30 mL) and washed with saturated brine (60 mL), dried over anhydrous Na₂SO₄, and concentrated in vacuo to give the crude, which was purified by column chromatography on silica gel (eluent: PE/EtOAc=10/1) to give (E)-N′-(5-bromopyrazin-2(1H)-ylidene)-2-(6-cyclopropyl-8-(3-methyl-2,4-dioxoimidazolidin-1-yl)imidazo[1,2-a]pyridin-2-yl)acetohydrazide (0.10 g, yield 18%) as a yellow oil. ESI-MS [M+H]+: 499.1

Synthesis of 1-(2-((6-bromo-[1,2,4]triazolo[4,3-a]pyrazin-3-yl)methyl)-6-cyclopropylimidazo[1,2-a]pyridin-8-yl)-3-methylimidazolidine-2,4-dione. A mixture of (E)-N′-(5-bromopyrazin-2(1H)-ylidene)-2-(6-cyclopropyl-8-(3-methyl-2,4-dioxoimidazolidin-1-yl)imidazo[1,2-a]pyridin-2-yl)acetohydrazide (25 mg, 0.050 mmol), C₂Cl₆ (30 mg, 0.13 mmol), PPh₃ (33 mg, 0.13 mmol), and Et₃N (0.050 mL, 0.36 mmol) in toluene (8 mL) was stirred at room temperature for 16 h. The combined organic layers were washed with brine (sat·aq·, 60 mL), dried over anhydrous Na₂SO₄, and concentrated in vacuo to give the crude, which was purified by column chromatography on silica gel (eluent: PE/EtOAc=15/1) to give 1-(2-((6-bromo-[1,2,4]triazolo[4,3-a]pyrazin-3-yl)methyl)-6-cyclopropylimidazo[1,2-a]pyridin-8-yl)-3-methylimidazolidine-2,4-dione (10 mg, 42%) as a yellow oil. ESI-MS [M+H]⁺: 481.1

Synthesis of (1S,2S)—N-(3-((6-cyclopropyl-8-(3-methyl-2,4-dioxoimidazolidin-1-yl)imidazo[1,2-a]pyridin-2-yl)methyl)-[1,2,4]triazolo[4,3-a]pyrazin-6-yl)-2-(4-methylpyrimidin-2-yl)cyclopropane-1-carboxamide (I-630). To a mixture of 1-(2-((6-bromo-[1,2,4]triazolo[4,3-a]pyrazin-3-yl)methyl)-6-cyclopropylimidazo[1,2-a]pyridin-8-yl)-3-methylimidazolidine-2,4-dione (0.10 g, 0.21 mmol), (1S,2S)-2-(4-methylpyrimidin-2-yl)cyclopropane-1-carboxamide (46 mg, 0.26 mmol), and Cs₂CO₃ (0.20 g, 0.61 mmol) in dioxane (8.0 mL) were added Pd₂(dba)₃ (20 mg, 0.022 mmol) and Xant-phos (1.6 g, 2.8 mmol). The reaction mixture was stirred at 60° C. for 1 h under N₂. The mixture was filtered through Celite® and the filter cake was washed with DCM/MeOH (10/1, 0.10 mL). The filtrate was concentrated in vacuo to give the crude, which was purified by silica gel chromatography (eluent: DCM/MeOH=50/1˜20/1) to give (1S,2S)—N-(3-((6-cyclopropyl-8-(3-methyl-2,4-dioxoimidazolidin-1-yl)imidazo[1,2-a]pyridin-2-yl)methyl)-[1,2,4]triazolo[4,3-a]pyrazin-6-yl)-2-(4-methylpyrimidin-2-yl)cyclopropane-1-carboxamide (50 mg, 41%) as a yellow solid. ESI-MS [M+H]+: 578.2. ¹H NMR (400 MHz, DMSO) δ 11.07 (s, 1H), 9.42 (s, 1H), 9.26 (s, 1H), 8.53 (d, J=5.1 Hz, 1H), 8.22 (s, 1H), 7.83 (s, 1H), 7.38 (d, J=1.4 Hz, 1H), 7.21 (d, J=5.1 Hz, 1H), 4.91 (d, J=3.4 Hz, 2H), 4.67 (s, 2H), 3.00 (s, 3H), 2.56-2.55 (m, 2H), 2.42 (s, 3H), 2.02-1.99 (m, 1H), 1.50-1.47 (m, 2H), 0.95-0.93 (m, 2H), 0.64-0.63 (m, 2H).

### Example 631

### Synthesis of (1S,2S)-2-(4-chloropyrimidin-2-yl)-N-(8-((6-cyclopropyl-8-(3-methyl-2,4-dioxoimidazolidin-1-yl)imidazo[1,2-a]pyridin-2-yl)methyl)-7,8-dihydro-6H-pyrimido[5,4-b][1,4]oxazin-2-yl)cyclopropane-1-carboxamide (I-631)

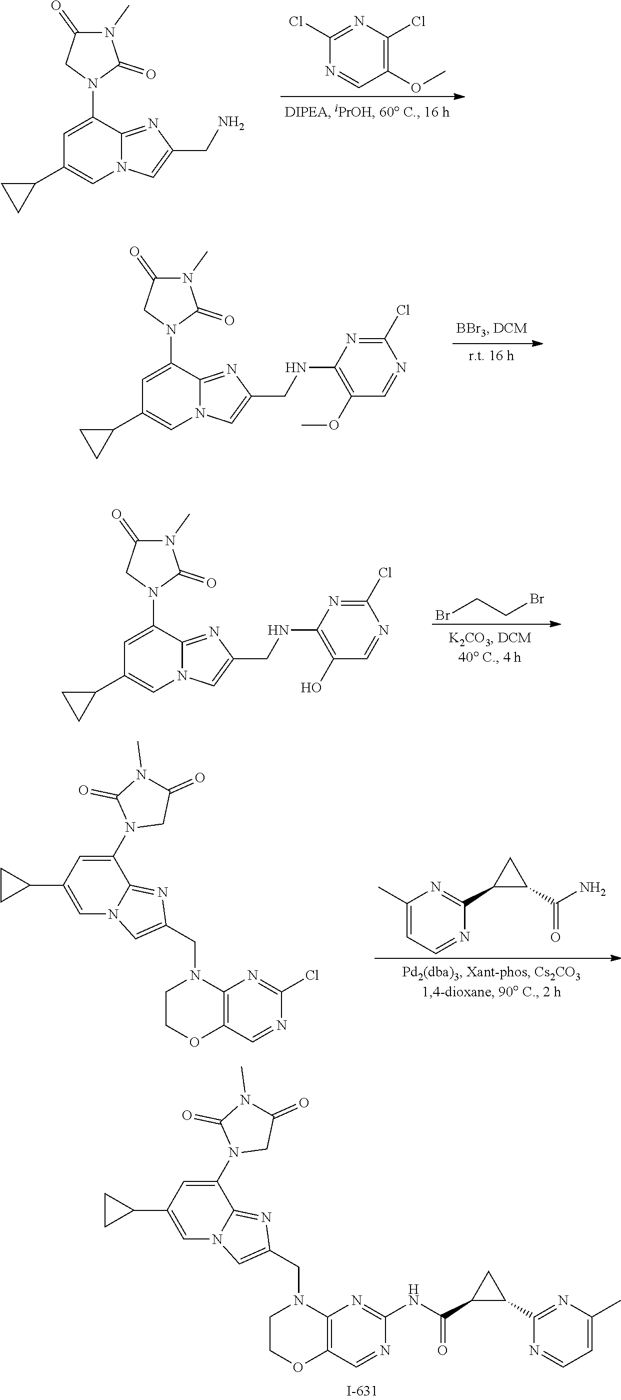

Synthesis of 1-(2-(((2-chloro-5-methoxypyrimidin-4-yl)amino)methyl)-6-cyclopropylimidazo[1,2-a] pyridin-8-yl)-3-methylimidazolidine-2,4-dione. A mixture of 2,4-dichloro-5-methoxypyrimidine (0.36 g, 2.0 mmol), 1-(2-(aminomethyl)-6-cyclopropylimidazo[1,2-a]pyridin-8-yl)-3-methylimidazolidine-2,4-dione (0.30 g, 1.0 mmol), and DIPEA (0.39 g, 3.0 mmol) in ⁱPrOH (10 mL) was stirred at 60° C. for 16 h. The mixture was concentrated in vacuo to get a crude, which was purified by preparative TLC (DCM/MeOH=20/1) to give 1-(2-(((2-chloro-5-methoxypyrimidin-4-yl)amino)methyl)-6-cyclopropylimidazo[1,2-a]pyridin-8-yl)-3-methylimidazolidine-2,4-dione (0.35 g, 79%) as a white solid. ESI-MS [M+H]⁺: 442.2.

Synthesis of 1-(2-(((2-chloro-5-hydroxypyrimidin-4-yl)amino)methyl)-6-cyclopropylimidazo[1,2-a] pyridin-8-yl)-3-methylimidazolidine-2,4-dione. To mixture of 1-(2-(((2-chloro-5-methoxypyrimidin-4-yl)amino)methyl)-6-cyclopropylimidazo[1,2-a]pyridin-8-yl)-3-methylimidazolidine-2,4-dione (0.22 g, 0.50 mmol) in dry DCM (10 mL) was added BBr₃ (1.3 g, 5.2 mmol) at 0° C. Then the mixture was stirred at room temperature for 16 h. The reaction mixture was quenched with saturated aq·NH₄Cl (20 mL) and extracted with EtOAc (3×30 mL). The combined organic layers were washed with saturated brine (30 mL), dried over anhydrous Na₂SO₄, and concentrated in vacuo to give the crude, which was purified by column chromatography on silica gel (eluent: DCM/MeOH=20/1) to give 1-(2-(((2-chloro-5-hydroxypyrimidin-4-yl)amino)methyl)-6-cyclopropylimidazo[1,2-a]pyridin-8-yl)-3-methylimidazolidine-2,4-dione (0.10 g, 47%) as a yellow solid. ESI-MS [M+H]⁺: 428.1.

Synthesis of 1-(2-((2-chloro-6,7-dihydro-8H-pyrimido[5,4-b][1,4]oxazin-8-yl)methyl)-6-cyclopropylimidazo [1,2-a]pyridin-8-yl)-3-methylimidazolidine-2,4-dione. A mixture of 1-(2-(((2-chloro-5-hydroxypyrimidin-4-yl)amino)methyl)-6-cyclopropylimidazo [1,2-a]pyridin-8-yl)-3-methylimidazolidine-2,4-dione (0.10 g, 0.23 mmol), 1,2-dibromoethane (65 mg, 0.35 mmol), and K₂CO₃ (95 mg, 0.69 mmol) in DCM (10.0 mL) was stirred at 40° C. for 4 h. The mixture was concentrated in vacuo to get a crude, which was purified by preparative TLC (DCM/MeOH=20/1) to give 1-(2-((2-chloro-6,7-dihydro-8H-pyrimido[5,4-b][1,4]oxazin-8-yl)methyl)-6-cyclopropyl imidazo[1,2-a]pyridin-8-yl)-3-methylimidazolidine-2,4-dione (40 mg, 38%) as a white solid. ESI-MS [M+H]⁺: 454.2.

Synthesis of (1S,2S)-2-(4-chloropyrimidin-2-yl)-N-(8-((6-cyclopropyl-8-(3-methyl-2,4-dioxoimidazolidin-1-yl)imidazo[1,2-a]pyridin-2-yl)methyl)-7,8-dihydro-6H-pyrimido[5,4-b][1,4]oxazin-2-yl)cyclopropane-1-carboxamide (I-631). To a mixture of 1-(2-((2-chloro-6,7-dihydro-8H-pyrimido[5,4-b][1,4]oxazin-8-yl)methyl)-6-cyclopropylimidazo[1,2-a]pyridin-8-yl)-3-methylimidazolidine-2,4-dione (40 mg, 0.088 mmol), 2-(4-methylpyrimidin-2-yl)cyclopropane-1-carboxamide (16 mg, 0.090 mmol), and Cs₂CO₃ (86 mg, 0.26 mmol) in dioxane (5.0 mL) were added Pd₂(dba)₃ (8.0 mg, 0.0087 mmol) and Xant-phos (9.5 mg, 0.02 mmol). After stirring at 90° C. for 2 h under N₂, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=15/1) to give (1S,2S)-2-(4-chloropyrimidin-2-yl)-N-(8-((6-cyclopropyl-8-(3-methyl-2,4-dioxoimidazolidin-1-yl)imidazo[1,2-a]pyridin-2-yl)methyl)-7,8-dihydro-6H-pyrimido[5,4-b][1,4]oxazin-2-yl)cyclopropane-1-carboxamide (22 mg, 42%) as a white solid. ESI-MS [M+H]⁺: 595.2, ¹H NMR (400 MHz, DMSO) δ 10.26 (s, 1H), 8.46 (d, J=5.1 Hz, 1H), 8.20 (s, 1H), 7.80 (s, 1H), 7.64 (s, 1H), 7.35 (d, J=1.3 Hz, 1H), 7.14 (d, J=5.0 Hz, 1H), 4.88 (s, 2H), 4.85-4.75 (m, 2H), 4.14 (t, J=4.3 Hz, 2H), 3.60 (s, 2H), 2.53-2.50 (m, 2H), 2.37 (s, 3H), 1.98-1.91 (m, 1H), 1.57-1.42 (m, 2H), 1.00-0.84 (m, 2H), 0.74-0.54 (m, 2H) ppm.

### Examples 632-634

### Synthesis of rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclo propane-1-carboxamide (I-632)

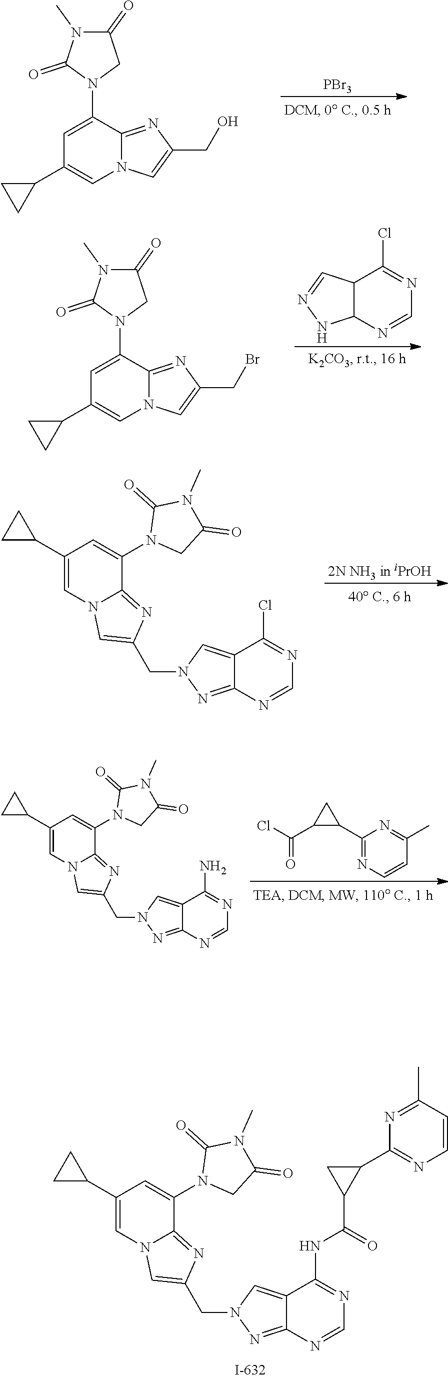

Synthesis of 1-(2-(bromomethyl)-6-cyclopropylimidazo[1,2-a]pyridin-8-yl)-3-methylimidazolidine-2,4-dione. To a mixture of 1-(6-cyclopropyl-2-(hydroxymethyl)imidazo[1,2-a]pyridin-8-yl)-3-methylimidazoli dine-2,4-dione (0.50 g, 1.7 mmol) in DCM (10 mL) was added PBr₃ (1.0 mL). After stirring at 0° C. for 0.5 h, the reaction mixture was quenched by saturated aq·NaHCO₃ (10 mL) and extracted by DCM (3×20 mL). The combined organic layers were concentrated in vacuo to afford 1-(2-(bromomethyl)-6-cyclopropylimidazo[1,2-a]pyridin-8-yl)-3-methylimidazolidine-2,4-dione (0.32 g, 52% yield) as a yellow oil. ESI-MS [M+H]+: 363.1.

Synthesis of 1-(2-((4-chloro-2H-pyrazolo[3,4-d]pyrimidin-2-yl)methyl)-6-cyclopropylimidazo[1,2-a]pyridin-8-yl)-3-methylimidazolidine-2,4-dione. A mixture of 1-(2-(bromomethyl)-6-cyclopropylimidazo[1,2-a]pyridin-8-yl)-3-methylimidazolidine-2,4-dione (0.32 g, 0.88 mmol), 4-chloro-3a,7a-dihydro-1H-pyrazolo[3,4-d]pyrimidine (0.14 g, 0.89 mmol), and K₂CO₃ (0.37 g, 2.7 mmol) in DMF (10 mL) was stirred at room temperature for 16 h under N₂. The reaction mixture was quenched with water (30 mL) and extracted with EtOAc (3×30 mL). The combined organic layers were washed with saturated brine (20 mL), dried over anhydrous Na₂SO₄, and concentrated in vacuo to give the crude product, which was purified by prep-HPLC to afford 1-(2-((4-chloro-2H-pyrazolo[3,4-d]pyrimidin-2-yl)methyl)-6-cyclopropylimidazo[1,2-a]pyridin-8-yl)-3-methylimidazolidine-2,4-dione (50 mg, 13% yield) as yellow solid. ESI-MS [M+H]+: 437.2.

Synthesis of 1-(2-((4-amino-2H-pyrazolo[3,4-d]pyrimidin-2-yl)methyl)-6-cyclopropylimidazo[1,2-a]pyridin-8-yl)-3-methylimidazolidine-2,4-dione. A solution of 1-(2-((4-chloro-2H-pyrazolo[3,4-d]pyrimidin-2-yl)methyl)-6-cyclopropylimidazo[1,2-a]pyridin-8-yl)-3-methylimidazolidine-2,4-dione (0.34 g, 0.78 mmol) in NH₃—ⁱPrOH (2 N, 10 mL) was stirred at 40° C. for 6 h under N₂. The reaction mixture was concentrated in vacuo to give a crude, which was purified by preparative TLC (eluent: DCM/MeOH=10/1) to afford 1-(2-((4-amino-2H-pyrazolo[3,4-d]pyrimidin-2-yl)methyl)-6-cyclopropylimidazo[1,2-a]pyridin-8-yl)-3-methylimidazolidine-2,4-dione (40 mg, 12% yield) as yellow solid. ESI-MS [M+H]+: 418.2.

Synthesis of rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide (I-632). A mixture of 1-(2-((4-amino-2H-pyrazolo[3,4-d]pyrimidin-2-yl)methyl)-6-cyclopropylimidazo [1,2-a]pyridin-8-yl)-3-methylimidazolidine-2,4-dione (50 mg, 0.12 mmol), rac-(1S*,2S*)-2-(4-methylpyrimidin-2-yl)cyclopropane-1-carbonyl chloride (26 mg, 0.13 mmol) and TEA (13 mg, 0.13 mmol) in DCM (0.5 mL) was stirred in a sealed tube. After degassing with N₂ for 1 min, the mixture was irradiated in a microwave reactor at 110° C. for 1 h. The reaction mixture was concentrated to give a crude, which was purified by preparative HPLC to afford rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide (16 mg, 23% yield) as a pale solid (mixture of enantiomers). ESI-MS [M+H]+: 578.2, ¹H NMR (400 MHz, DMSO) δ 11.56 (s, 1H), 9.01 (s, 1H), 8.57-8.54 (m, 2H), 8.30 (d, J=1.3 Hz, 1H), 7.94 (s, 1H), 7.39 (d, J=1.5 Hz, 1H), 7.23 (d, J=5.1 Hz, 1H), 5.79 (s, 2H), 4.88 (s, 2H), 2.96 (s, 3H), 2.82-2.78 (m, 1H), 2.69-2.65 (m, 1H), 2.43 (s, 3H), 1.98-1.94 (m, 1H), 1.68-1.61 (m, 2H), 0.98-0.93 (m, 2H), 0.68-0.64 (m, 2H).

The mixture was separated using SFC 80 (Daicel CHIRALPAK OD-H 250 mm×20 mm I.D. CO₂/[MeOH/ACN=1/1 (0.2% MA)]=55/45. 50 g/min, 35° C.) to give two enantiomers: (1S,2S)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide and (1R,2R)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide

First eluting isomer (I-633): ESI-MS [M+H]+: 578.2. ¹H NMR (400 MHz, MeOD) δ 9.03 (s, 1H), 8.54 (s, 1H), 8.48-8.47 (m, 1H), 8.19 (d, J=0.9 Hz, 1H), 7.92 (s, 1H), 7.38 (d, J=1.4 Hz, 1H), 7.18 (d, J=1.5 Hz, 1H), 5.76 (s, 2H), 4.76 (d, J=1.9 Hz, 2H), 3.08 (s, 3H), 2.81-2.77 (m, 1H), 2.70-2.66 (m, 1H), 2.48 (s, 3H), 1.99-1.94 (m, 1H), 1.80-1.72 (m, 2H), 1.02-0.97 (m, 2H), 0.76-0.72 (m, 2H). RT=1.74 min, 100.0% e.e.

Second eluting isomer (I-634): ESI-MS [M+H]+: 578.2. ¹H NMR (400 MHz, DMSO) δ 11.56 (s, 1H), 9.00 (s, 1H), 8.57-8.54 (m, 2H), 8.30 (d, J=1.5 Hz, 1H), 7.94 (s, 1H), 7.39 (d, J=1.5 Hz, 1H), 7.23 (d, J=1.5 Hz, 1H), 5.79 (s, 2H), 4.88 (s, 2H), 2.96 (s, 3H), 2.82-2.78 (m, 1H), 2.69-2.64 (m, 1H), 2.43 (s, 3H), 1.98-1.94 (m, 1H), 1.68-1.61 (m, 2H), 0.98-0.93 (m, 2H), 0.68-0.64 (m, 2H). RT=2.87 min, 100.0% e.e.

### Example 635

### Synthesis of (1S,2S)—N-(1-((6-cyclopropyl-8-(3-methyl-2,4-dioxoimidazolidin-1-yl)imidazo[1,2-a]pyridin-2-yl)methyl)-1H-[1,2,3]triazolo[4,5-c]pyridin-6-yl)-2-(4-methylpyrimidin-2-yl)cyclopropane-1-carboxamide (I-635)

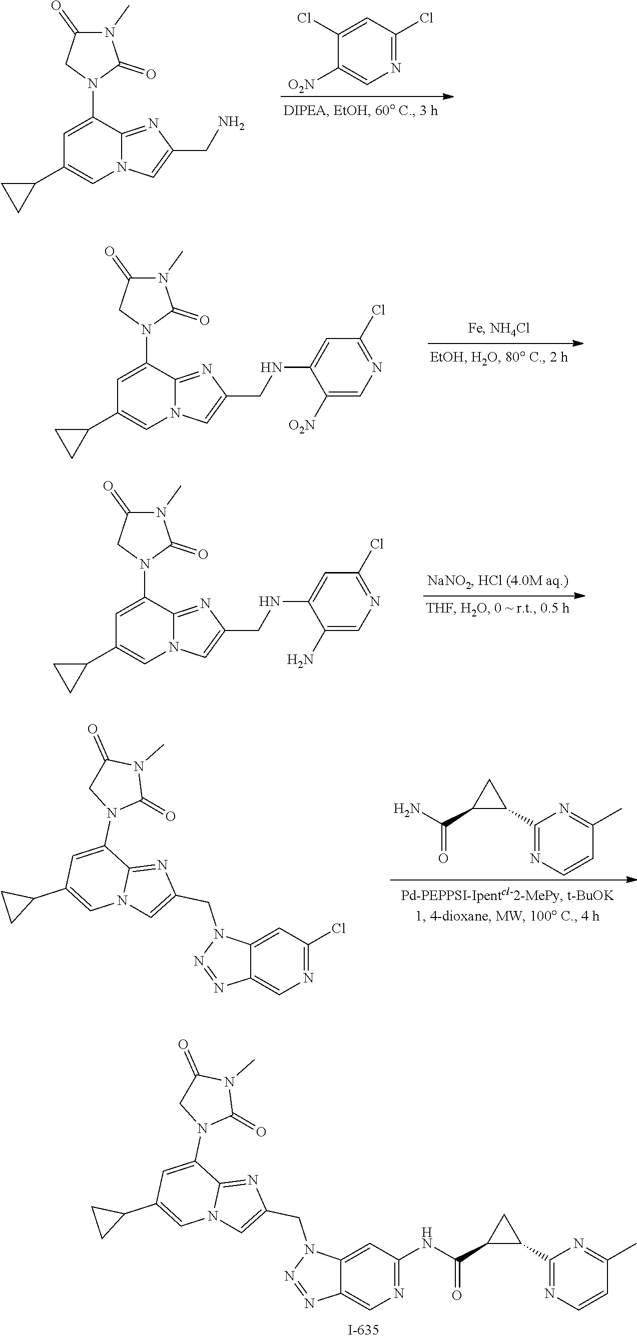

Synthesis of 1-(2-(((2-chloro-5-nitropyridin-4-yl)amino)methyl)-6-cyclopropylimidazo[1,2-a]pyridin-8-yl)-3-methylimidazolidine-2,4-dione. To a mixture of 1-(2-(aminomethyl)-6-cyclopropylimidazo[1,2-a]pyridin-8-yl)-3-methylimidazolidine-2,4-dione (0.80 g, 2.7 mmol) and 2,4-dichloro-5-nitropyridine (0.62 g, 3.2 mmol) in EtOH (10 mL) was added DIPEA (1.0 g, 8.0 mmol). After stirring at 60° C. for 3 h, the reaction mixture was concentrated in vacuo to give the crude, which was purified by column chromatography on silica gel (eluent: EtOAc/PE=0˜100%, then, MeOH/DCM=0˜20%) to afford 1-(2-(((2-chloro-5-nitropyridin-4-yl)amino)methyl)-6-cyclopropylimidazo[1,2-a]pyridin-8-yl)-3-methylimidazolidine-2,4-dione (1.0 g, yield 81%) as yellow solid. ESI-MS [M+H]+: 456.2.

Synthesis of 1-(2-(((5-amino-2-chloropyridin-4-yl)amino)methyl)-6-cyclopropylimidazo[1,2-a]pyridin-8-yl)-3-methylimidazolidine-2,4-dione. To a mixture of 1-(2-(((2-chloro-5-nitropyridin-4-yl)amino)methyl)-6-cyclopropylimidazo[1,2-a]pyridin-8-yl)-3-methylimidazolidine-2,4-dione (1.0 g, 2.2 mmol) and Fe powder (0.49 g, 8.8 mmol) in EtOH (18 mL) was added a solution of NH₄Cl (0.47 g, 8.8 mmol) in water (2.0 mL). After stirring at 80° C. for 2 h, the reaction mixture was filtered by Celite©. The filtered cake was washed with DCM/MeOH (v/v=3/1, 10 mL×3). The filtrate was concentrated in vacuo to afford 1-(2-(((5-amino-2-chloropyridin-4-yl)amino)methyl)-6-cyclopropylimidazo[1,2-a]pyridin-8-yl)-3-methylimidazolidine-2,4-dione (0.80 g, yield 85%) as yellow solid, which was used without further purification. ESI-MS [M+H]+: 426.2.

Synthesis of 1-(2-((6-chloro-1H-[1,2,3]triazolo[4,5-c]pyridin-1-yl)methyl)-6-cyclopropylimidazo[1,2-a]pyridin-8-yl)-3-methylimidazolidine-2,4-dione. To a mixture of 1-(2-(((5-amino-2-chloropyridin-4-yl)amino)methyl)-6-cyclopropylimidazo[1,2-a]pyridin-8-yl)-3-methylimidazolidine-2,4-dione (0.80 g, 1.9 mmol) and HCl (2.4 mL, 4 N HCl in dioxane, 9.6 mmol) in THF (10 mL) was added of a solution of NaNO₂ (0.39 g, 5.7 mmol) in water (5.0 mL) at 0° C. After stirring at room temperature for 0.5 h, the reaction mixture was neutralized with NH₄OH (5.0 mL), and then extracted with DCM (3×10 mL). The combined organic phase was washed with saturated brine (15 mL), dried over Na₂SO₄, and concentrated in vacuo to get a residue, which was purified by column chromatography on silica gel (eluent: EtOAc/PE=0˜75%) to afford 1-(2-((6-chloro-1H-[1,2,3]triazolo[4,5-c]pyridin-1-yl)methyl)-6-cyclopropylimidazo [1,2-a]pyridin-8-yl)-3-methylimidazolidine-2,4-dione (0.20 g, yield 24%) as brown solid. ESI-MS [M+H]+: 437.2.

Synthesis of (1S,2S)—N-(1-((6-cyclopropyl-8-(3-methyl-2,4-dioxoimidazolidin-1-yl)imidazo[1,2-a]pyridin-2-yl)methyl)-1H-[1,2,3]triazolo[4,5-c]pyridin-6-yl)-2-(4-methylpyrimidin-2-yl)cyclopropane-1-carboxamide (I-635). To a mixture of 1-(2-((6-chloro-1H-[1,2,3]triazolo[4,5-c]pyridin-1-yl)methyl)-6-cyclopropylimidazo[1,2-a]pyridin-8-yl)-3-methylimidazolidine-2,4-dione (90 mg, 0.21 mmol) and (1S,2S)-2-(4-methylpyrimidin-2-yl)cyclopropane-1-carboxamide (40 mg, 0.23 mmol) in dioxane (10 mL) was added Pd-PEPPSI-IpentCl-2-MePy (69 mg, 0.082 mmol) and t-BuOK (92 mg, 0.82 mmol). After stirring at 100° C. for 4 h in a microwave reactor, the mixture was diluted with DCM (20 mL) and filtered through Celite®. The filtered cake was washed with DCM/MeOH (v/v=3/1, 3×10 mL). The filtrates were concentrated in vacuo and purified by column chromatography on silica gel (eluent: EtOAc/PE=0˜100%) to afford crude product, which was purified by preparative TLC again (eluent: PE:EA=1:3) to afford (1S,2S)—N-(1-((6-cyclopropyl-8-(3-methyl-2,4-dioxoimidazolidin-1-yl)imidazo[1,2-a]pyridin-2-yl)methyl)-1H-[1,2,3]triazolo[4,5-c]pyridin-6-yl)-2-(4-methylpyrimidin-2-yl) cyclopropane-1-carboxamide (33 mg, yield 27%) as white solid. ESI-MS [M+H]+: 578.3. ¹H NMR (400 MHz, DMSO) δ 11.14 (s, 1H), 9.19 (d, J=1.0 Hz, 1H), 8.69 (d, J=0.9 Hz, 1H), 8.53 (d, J=5.1 Hz, 1H), 8.25 (d, J=1.0 Hz, 1H), 7.94 (s, 1H), 7.40 (d, J=1.4 Hz, 1H), 7.21 (d, J=5.1 Hz, 1H), 6.03 (s, 2H), 4.93-4.83 (m, 2H), 2.99 (s, 3H), 2.71-2.67 (m, 1H), 2.56-2.53 (m, 1H), 2.42 (s, 3H), 1.96-1.91 (m, 1H), 1.56-1.49 (m, 2H), 0.96-0.91 (m, 2H), 0.66-0.62 (m, 2H).

### Examples 636-637

### Synthesis of (1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide (I-636) and (1S,2S)—N-(6-cyclopropyl-4-(((6-cyclopropyl-8-(3-(2-hydroxyethyl)-2,4-dioxoimidazolidin-1-yl)imidazo[1,2-a]pyridin-2-yl)methyl)(methyl)amino)pyridin-2-yl)-2-(4-methylpyrimidin-2-yl)cyclopropane-1-carboxamide (I-637)

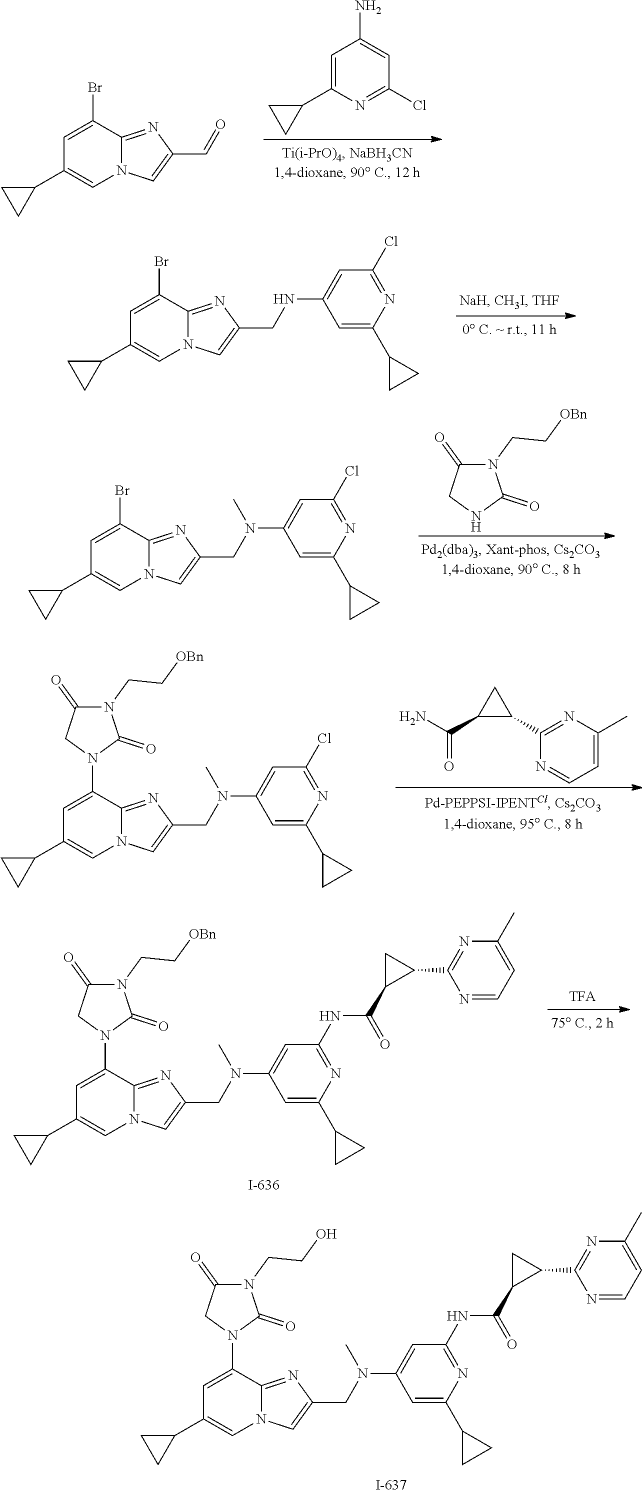

Synthesis of N-((8-bromo-6-cyclopropylimidazo[1,2-a]pyridin-2-yl)methyl)-2-chloro-6-cyclopropylpyridin-4-amine. A mixture of 8-bromo-6-cyclopropylimidazo[1,2-a]pyridine-2-carbaldehyde (0.26 g, 0.98 mmol), 2-chloro-6-cyclopropylpyridin-4-amine (0.17 g, 1.0 mmol) and Ti(i-PrO)₄ (1.4 g, 4.9 mmol) in dioxane (6.0 mL) was stirred at 90° C. for 10 h under N₂. After cooling to room temperature, the mixture was diluted with MeOH (0.50 mL) and NaBH₃CN (0.32 g, 5.1 mmol) was added. Then the mixture was stirred for another 1 h at room temperature. The resulting mixture was quenched with water (50 mL) and extracted with EtOAc (3×15 mL). The combined organic layers were concentrated in vacuo and purified by column chromatography on silica gel (eluent: DCM/MeOH=0˜5%) to give N-((8-bromo-6-cyclopropylimidazo [1,2-a]pyridin-2-yl)methyl)-2-chloro-6-cyclopropylpyridin-4-amine (0.20 g, 49%) as a light yellow solid. ESI-MS [M+H]⁺: 416.9.

Synthesis of N-((8-bromo-6-cyclopropylimidazo[1,2-a]pyridin-2-yl)methyl)-2-chloro-6-cyclopropyl-N-methylpyridin-4-amine. To a solution of N-((8-bromo-6-cyclopropylimidazo[1,2-a]pyridin-2-yl)methyl)-2-chloro-6-cyclopropylpyridin-4-amine (0.20 g, 0.48 mmol) in THF (2.0 mL) was added NaH (20 mg, 0.50 mmol) at 0° C. After stirring at room temperature for 1 h under N₂, CH₃I (124 mg, 0.87 mmol) was added and continued stirring for another 10 h at room temperature. The reaction mixture was quenched with water and then concentrated to give the crude product, which was purified by preparative TLC (eluent: DCM/MeOH=20:1) to give N-((8-bromo-6-cyclopropylimidazo[1,2-a]pyridin-2-yl)methyl)-2-chloro-6-cyclopropyl-N-methylpyridin-4-amine (0.10 g, 48%) as a white solid. ESI-MS [M+H]+: 431.0.

Synthesis of 3-(2-(benzyloxy)ethyl)-1-(2-(((2-chloro-6-cyclopropylpyridin-4-yl)(methyl)amino)methyl)-6-cyclopropylimidazo[1,2-a]pyridin-8-yl)imidazolidine-2,4-dione. A mixture of N-((8-bromo-6-cyclopropylimidazo[1,2-a]pyridin-2-yl)methyl)-2-chloro-6-cyclopropyl-N-methylpyridin-4-amine (0.10 g, 0.23 mmol), 3-(2-(benzyloxy)ethyl)imidazolidine-2,4-dione (0.11 g, 0.47 mmol), Pd₂(dba)₃ (21 mg, 0.023 mmol), Xant-phos (13 mg, 0.022 mmol), and Cs₂CO₃ (0.12 g, 0.37 mmol) in dioxane (3.0 mL) was stirred at 90° C. for 8 h under N₂. The reaction mixture was concentrated in vacuo and purified by preparative TLC (eluent: DCM/MeOH=20:1) to give 3-(2-(benzyloxy)ethyl)-1-(2-(((2-chloro-6-cyclopropylpyridin-4-yl)(methyl)amino)methyl)-6-cyclopropylimidazo[1,2-a]pyridin-8-yl)imidazolidine-2,4-dione (80 mg, 59%) as a light yellow solid. ESI-MS [M+H]+: 585.2.

Synthesis of (1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide (I-636). A mixture 3-(2-(benzyloxy)ethyl)-1-(2-(((2-chloro-6-cyclopropylpyridin-4-yl)(methyl)amino)-methyl)-6-cyclopropylimidazo[1,2-a]pyridin-8-yl)imidazolidine-2,4-dione (80 mg, 0.14 mmol), (1S,2S)-2-(4-methylpyrimidin-2-yl)cyclopropane-1-carboxamide (29 mg, 0.16 mmol), Pd-PEPPSI-IPENTCl (13 mg, 0.015 mmol), and Cs₂CO₃ (0.16 g, 0.49 mmol) in dioxane (3.0 mL) was stirred at 95° C. for 8 h. The reaction mixture was concentrated in vacuo and purified by preparative TLC (eluent: DCM/MeOH=15:1) to give (1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide (43 mg, 42%) as a light yellow solid. ESI-MS [M+H]+: 726.3. ¹H NMR (400 MHz, DMSO) δ 10.18 (s, 1H), 8.47 (d, J=5.1 Hz, 1H), 8.20 (s, 1H), 7.61 (s, 1H), 7.34-7.20 (m, 7H), 7.15 (d, J=5.1 Hz, 1H), 6.41 (d, J=2.0 Hz, 1H), 4.91 (s, 2H), 4.60 (s, 2H), 4.47 (s, 2H), 3.68 (t, J=5.4 Hz, 2H), 3.61 (t, J=5.3 Hz, 2H), 3.03 (s, 3H), 2.63-2.50 (m, 2H), 2.37 (s, 3H), 1.93-1.86 (m, 1H), 1.81-1.78 (m, 1H), 1.45-1.42 (m, 2H), 0.92-0.87 (m, 2H), 0.77-0.72 (m, 4H), 0.62-0.58 (m, 2H).

Synthesis of (1S,2S)—N-(6-cyclopropyl-4-(((6-cyclopropyl-8-(3-(2-hydroxyethyl)-2,4-dioxoimidazolidin-1-yl)imidazo[1,2-a]pyridin-2-yl)methyl)(methyl)amino)pyridin-2-yl)-2-(4-methylpyrimidin-2-yl)cyclopropane-1-carboxamide (I-637). A solution of (1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide (43 mg, 0.059 mmol) in TFA (3.0 mL) was stirred at 75° C. for 2 h. The reaction mixture was concentrated in vacuo and purified by preparative TLC (eluent: DCM/MeOH=10:1) to give (1S,2S)—N-(6-cyclopropyl-4-(((6-cyclopropyl-8-(3-(2-hydroxyethyl)-2,4-dioxoimidazolidin-1-yl)imidazo[1,2-a]pyridin-2-yl)methyl)(methyl)amino)pyridin-2-yl)-2-(4-methylpyrimidin-2-yl)cyclopropane-1-carboxamide (20 mg, 53%) as a light yellow solid. ESI-MS [M+H]+: 636.2. ¹H NMR (400 MHz, DMSO) δ 10.19 (s, 1H), 8.48 (d, J=5.1 Hz, 1H), 8.19 (d, J=0.9 Hz, 1H), 7.61 (s, 1H), 7.35-7.33 (m, 2H), 7.16 (d, J=5.1 Hz, 1H), 6.42 (d, J=2.0 Hz, 1H), 4.90 (s, 2H), 4.87-4.83 (m, 1H), 4.61 (s, 2H), 3.56-3.52 (m, 4H), 3.05 (s, 3H), 2.64-2.55 (m, 2H), 2.38 (s, 3H), 1.93-1.88 (m, 1H), 1.82-1.78 (m, 1H), 1.46-1.43 (m, 2H), 0.91-0.89 (m, 2H), 0.78-0.73 (m, 4H), 0.62-0.60 (m, 2H).

### Example 638

### Synthesis of (1S,2S)—N-(6-(((6-cyclopropyl-8-(3-methyl-2,4-dioxoimidazolidin-1-yl)imidazo[1,2-a]pyridin-2-yl)methyl)thio)-2-methylpyrimidin-4-yl)-2-(4-methylpyrimidin-2-yl)cyclopropane-1-carboxamide (I-638)

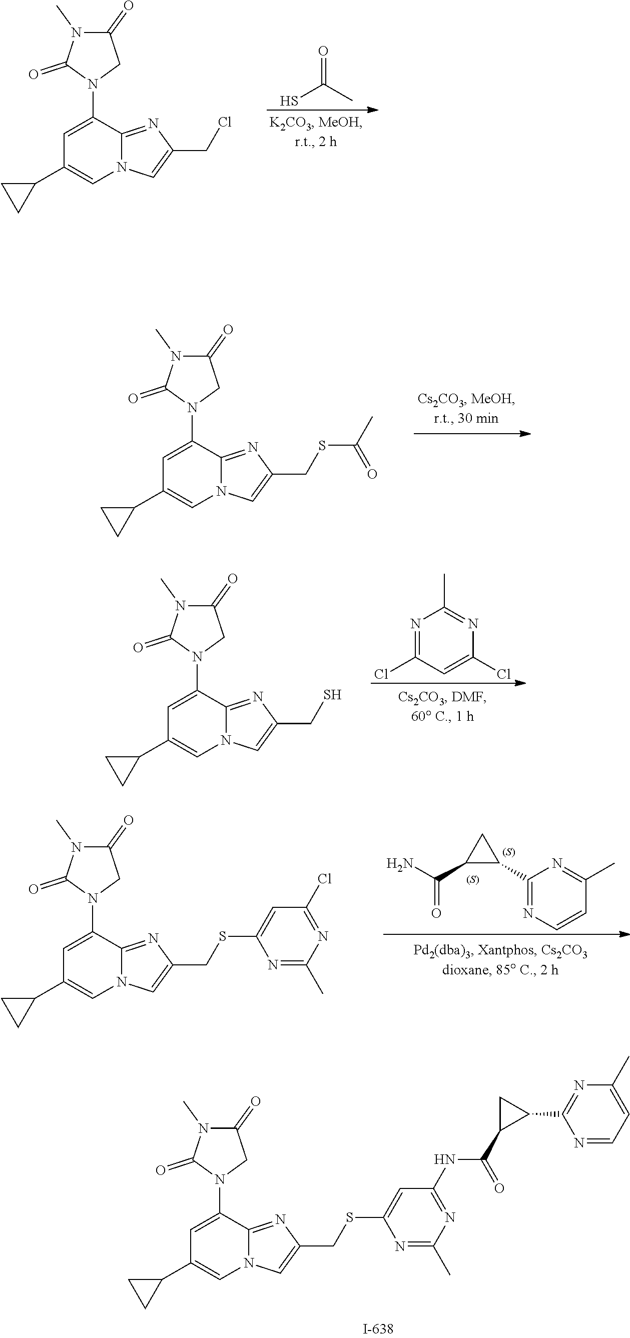

Synthesis of S-((6-cyclopropyl-8-(3-methyl-2,4-dioxoimidazolidin-1-yl)imidazo[1,2-a]pyridin-2-yl)methyl) ethanethioate. A mixture of 1-(2-(chloromethyl)-6-cyclopropylimidazo[1,2-a]pyridin-8-yl)-3-methylimidazolidine-2,4-dione (0.41 g, 1.3 mmol), ethanethioic S-acid (0.19 g, 2.5 mmol) and K₂CO₃ (0.35 g, 2.5 mmol) in MeOH (15 mL) was stirred at room temperature for 2 h. The reaction mixture was concentrated in vacuo, diluted in water (40 mL), and extracted with EtOAc (3×40 ml). The combined organics were washed with brine (40 mL), dried over Na₂SO₄, and concentrated in vacuo to give the crude product, which was purified by silica gel column chromatography (elute: DCM/MeOH=25/1) to give S-((6-cyclopropyl-8-(3-methyl-2,4-dioxoimidazolidin-1-yl)imidazo[1,2-a]pyridin-2-yl)methyl) ethanethioate (250 mg, 52%) as a yellow solid. ESI-MS [M+H]⁺: 359.1.

Synthesis of 1-(6-cyclopropyl-2-(mercaptomethyl)imidazo[1,2-a]pyridin-8-yl)-3-methylimidazolidine-2,4-dione. To a stirred solution of S-((6-cyclopropyl-8-(3-methyl-2,4-dioxoimidazolidin-1-yl)imidazo[1,2-a]pyridin-2-yl)methyl) ethanethioate (230 mg, 0.64 mmol) in MeOH (10 mL) was added Cs₂CO₃ (0.42 g, 1.3 mmol). After stirring at room temperature for 30 min, the reaction mixture was concentrated in vacuo, diluted in water (30 mL), and extracted with EtOAc (2×30 mL). The combined organics were washed with brine (40 mL), dried over Na₂SO₄, concentrated, and dried in vacuo to give 1-(6-cyclopropyl-2-(mercaptomethyl)imidazo[1,2-a]pyridin-8-yl)-3-methylimidazolidine-2,4-dione (220 mg, quant.) as a yellow solid. ESI-MS [M+H]⁺: 317.1.

Synthesis of 1-(2-(((6-chloro-2-methylpyrimidin-4-yl)thio)methyl)-6-cyclopropylimidazo[1,2-a]pyridin-8-yl)-3-methylimidazolidine-2,4-dione. A mixture of 1-(6-cyclopropyl-2-(mercaptomethyl)imidazo[1,2-a]pyridin-8-yl)-3-methylimidazolidine-2,4-dione (220 mg, 0.70 mmol), 4,6-dichloro-2-methylpyrimidine (230 mg, 1.4 mmol), and Cs₂CO₃ (450 mg, 1.4 mmol) in DMF (10 mL) was stirred at 60° C. for 1 h. The reaction mixture was poured into water (40 mL) and extracted with EtOAc (2×40 mL). The combined organics were washed with brine (40 mL), dried over Na₂SO₄, concentrated in vacuo, and purified by silica gel column chromatography (elute: PE/EtOAc=2/1) to give 1-(2-(((6-chloro-2-methylpyrimidin-4-yl)thio)methyl)-6-cyclopropylimidazo[1,2-a]pyridin-8-yl)-3-methylimidazolidine-2,4-dione (250 mg, 81%) as a yellow solid. ESI-MS [M+H]⁺: 443.1.

Synthesis of (1S,2S)—N-(6-(((6-cyclopropyl-8-(3-methyl-2,4-dioxoimidazolidin-1-yl)imidazo[1,2-a]pyridin-2-yl)methyl)thio)-2-methylpyrimidin-4-yl)-2-(4-methylpyrimidin-2-yl)cyclopropane-1-carboxamide (I-638). A mixture of 1-(2-(((6-chloro-2-methylpyrimidin-4-yl)thio)methyl)-6-cyclopropylimidazo[1,2-a]pyridin-8-yl)-3-methylimidazolidine-2,4-dione (60 mg, 0.14 mmol), (1S,2S)-2-(4-methylpyrimidin-2-yl)cyclopropane-1-carboxamide (29 mg, 0.16 mmol), Pd₂(dba)₃ (12 mg, 0.014 mmol), Xantphos (16 mg, 0.027 mmol), and Cs₂CO₃ (0.13 g, 0.41 mmol) in 1,4-dioxane (5.0 mL) was stirred at 85° C. for 2 h under N₂. The reaction mixture was diluted in DCM/MeOH (20 mL, 10/1), filtered, and the filtrate was concentrated in vacuo and purified by preparative TLC (DCM/MeOH=15/1) to give (1S,2S)—N-(6-(((6-cyclopropyl-8-(3-methyl-2,4-dioxoimidazolidin-1-yl)imidazo[1,2-a]pyridin-2-yl)methyl)thio)-2-methylpyrimidin-4-yl)-2-(4-methylpyrimidin-2-yl)cyclopropane-1-carboxamide (35 mg, 44%) as a white solid. ESI-MS [M+H]+: 584.2. ¹H NMR (400 MHz, DMSO) δ 11.20 (s, 1H), 8.53 (d, J=5.1 Hz, 1H), 8.24 (d, J=1.2 Hz, 1H), 7.91 (s, 1H), 7.84 (s, 1H), 7.37 (d, J=1.5 Hz, 1H), 7.21 (d, J=5.1 Hz, 1H), 4.89 (s, 2H), 4.48 (s, 2H), 2.97 (s, 3H), 2.65-2.60 (m, 1H), 2.57-2.55 (m, 1H), 2.49 (s, 3H), 2.41 (s, 3H), 1.99-1.91 (m, 1H), 1.58-1.49 (m, 2H), 0.98-0.90 (m, 2H), 0.69-0.61 (m, 2H).

### Example 639

### Synthesis of (1S,2S)—N-(2-(((6-cyclopropyl-8-(3-methyl-2,4-dioxoimidazolidin-1-yl)imidazo[1,2-a]pyridin-2-yl)methyl)amino)-6-methylpyridin-4-yl)-2-(4-methylpyrimidin-2-yl)cyclopropane-1-carboxamide (I-639)

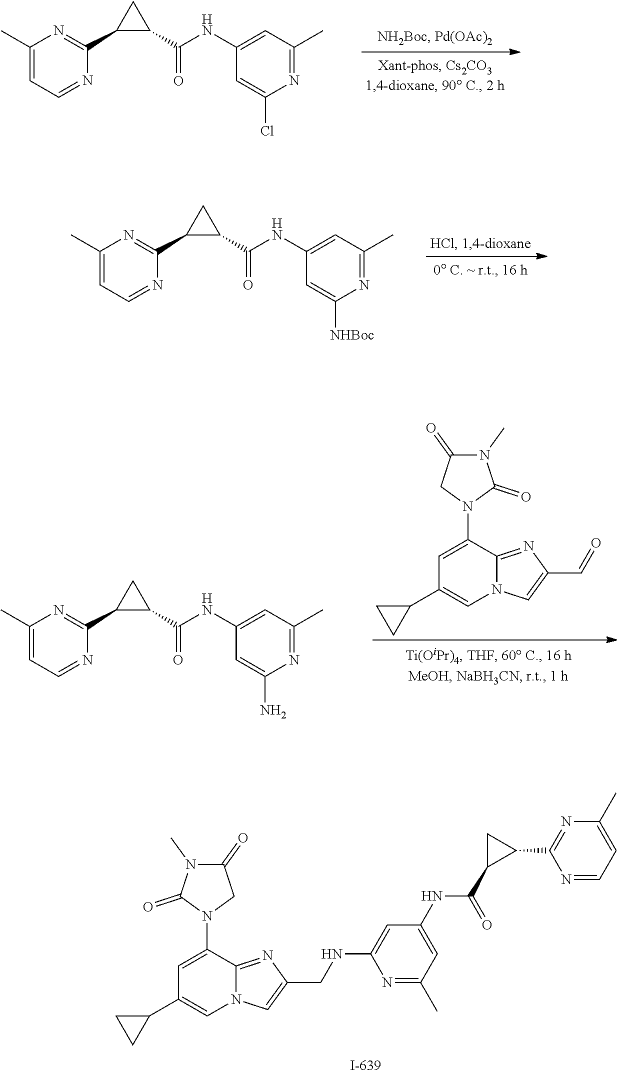

Synthesis of tert-butyl (6-methyl-4-((1S,2S)-2-(4-methylpyrimidin-2-yl)cyclopropane-1-carboxamido)pyridin-2-yl)carbamate. To a solution of (1S,2S)—N-(2-chloro-6-methylpyridin-4-yl)-2-(4-methylpyrimidin-2-yl)cyclopropane-1-carboxamide (0.15 g, 0.50 mmol) in dioxane (5.0 mL) was added NH₂Boc (0.12 g, 1.0 mmol), Cs₂CO₃ (0.49 g, 1.5 mmol), Xant-phos (48 mg, 0.083 mmol), and Pd(OAc)₂ (22 mg, 0.098 mmol) under N₂. After stirring at 90° C. for 2 h, the reaction mixture was concentrated in vacuo to give the crude, which was purified by column chromatography on silica gel (eluent: EtOAc/PE=1/1) to give tert-butyl (6-methyl-4-((1S,2S)-2-(4-methylpyrimidin-2-yl)cyclopropane-1-carboxamido)pyridin-2-yl)carbamate (0.11 g, 57%) as a white solid. ESI-MS [M+H]⁺: 384.2.

Synthesis of (1S,2S)—N-(2-amino-6-methylpyridin-4-yl)-2-(4-methylpyrimidin-2-yl)cyclopropane-1-carboxamide. To a solution of tert-butyl (6-methyl-4-((1S,2S)-2-(4-methylpyrimidin-2-yl)cyclopropane-1-carboxamido)pyridin-2-yl)carbamate (0.11 g, 0.29 mmol) in dioxane (3.0 mL) was added HCl (2.0 mL, 4 N HCl in 1, 4-dioxane, 8.0 mmol). After stirring at room temperature for 16 h, the reaction mixture was neutralized by adding NH₃ in MeOH (5.0 mL, 4 N, 20 mmol). The mixture was concentrated in vacuo and purified by preparative HPLC (eluent: MeOH/DCM=1/10) to give (1S,2S)—N-(2-amino-6-methylpyridin-4-yl)-2-(4-methylpyrimidin-2-yl)cyclopropane-1-carboxamide (60 mg, 73%) as a yellow solid. ESI-MS [M+H]⁺: 284.2.

Synthesis of (1S,2S)—N-(2-(((6-cyclopropyl-8-(3-methyl-2,4-dioxoimidazolidin-1-yl)imidazo[1,2-a]pyridin-2-yl)methyl)amino)-6-methylpyridin-4-yl)-2-(4-methylpyrimidin-2-yl)cyclopropane-1-carboxamide (I-639). To a solution of (1S,2S)—N-(2-amino-6-methylpyridin-4-yl)-2-(4-methylpyrimidin-2-yl) cyclopropane-1-carboxamide (60 mg, 0.21 mmol) in THF (5.0 mL) was added 6-cyclopropyl-8-(3-methyl-2,4-dioxoimidazolidin-1-yl)imidazo[1,2-a]pyridine-2-carbaldehyde (63 mg, 0.21 mmol) and Ti(OⁱPr)₄ (0.30 g, 1.1 mmol) at room temperature under N₂. After stirring at 60° C. for 16 h, NaBH₃CN (40 mg, 0.64 mmol) and MeOH (1.0 mL) were added and the resulting mixture was stirred at room temperature for another 1 h. The reaction was quenched with saturated aq·NH₄Cl (5.0 mL) and extracted with EtOAc/MeOH (10/1, 3×50 mL). The combined organic layers were concentrated and purified by preparative HPLC (eluent: MeOH/DCM=1/10) to give (1S,2S)—N-(2-(((6-cyclopropyl-8-(3-methyl-2,4-dioxoimidazolidin-1-yl)imidazo[1,2-a]pyridin-2-yl)methyl)amino)-6-methylpyridin-4-yl)-2-(4-methylpyrimidin-2-yl)cyclopropane-1-carboxamide (50 mg, 42%) as a white solid. ESI-MS [M+H]+: 566.2. ¹H NMR (400 MHz, DMSO) δ 10.34 (s, 1H), 8.52 (d, J=5.1 Hz, 1H), 8.25 (d, J=1.1 Hz, 1H), 7.68 (s, 1H), 7.32 (d, J=1.4 Hz, 1H), 7.21 (d, J=5.1 Hz, 1H), 6.96 (s, 1H), 6.80 (s, 1H), 6.56 (s, 1H), 4.91 (s, 2H), 4.50 (d, J=5.7 Hz, 2H), 2.97 (s, 3H), 2.52 (s, 2H), 2.41 (s, 3H), 2.22 (s, 3H), 1.96-1.90 (m, 1H), 1.51-1.48 (m, 2H), 0.96-0.91 (m, 2H), 0.67-0.61 (m, 2H).

### Example 640

### Synthesis of (1S,2S)—N-(5-(((6-cyclopropyl-8-(3-methyl-2,4-dioxoimidazolidin-1-yl)imidazo[1,2-a]pyridin-2-yl)methyl)amino)-6-(trifluoromethyl)pyridazin-3-yl)-2-(4-methylpyrimidin-2-yl)cyclopropane-1-carboxamide (I-640)

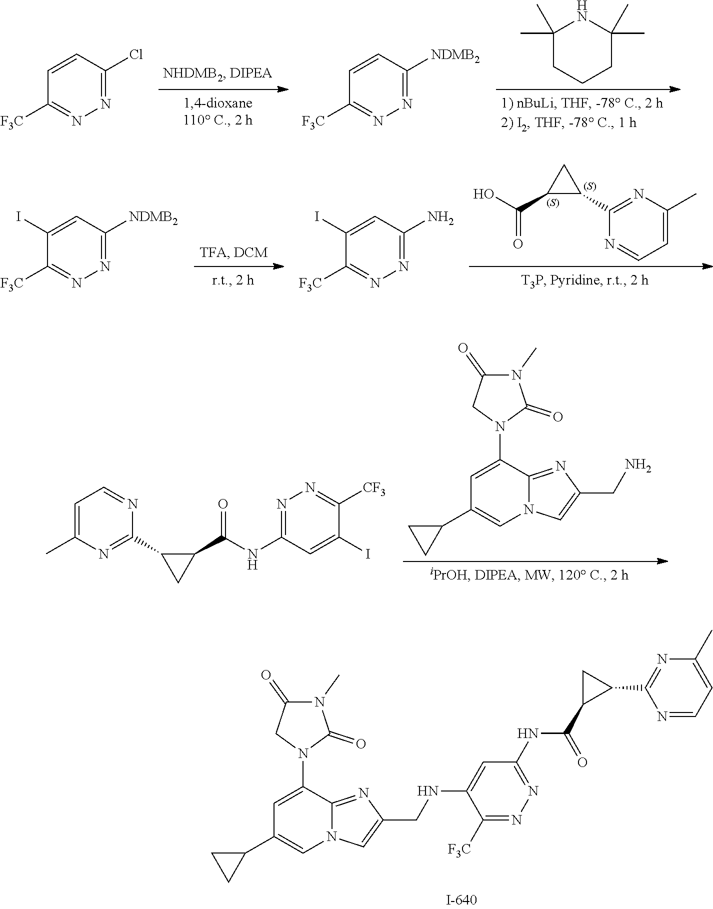

Synthesis of N,N-bis(2,4-dimethoxybenzyl)-6-(trifluoromethyl)pyridazin-3-amine. A mixture of 3-chloro-6-(trifluoromethyl)pyridazine (2.0 g, 11 mmol), bis(3,4-dimethoxybenzyl) amine (5.2 g, 16 mmol), and DIPEA (4.3 g, 33 mmol) in dioxane (30 mL) was stirred under N₂ at 110° C. for 2 h. After cooling to room temperature, the reaction was quenched with saturated aq·NaHCO₃ (100 mL) and extracted with EtOAc (3×50 mL). The combined organic layers were washed with saturated brine (50 mL), dried over Na₂SO₄ and concentrated in vacuo to get a residue, which was purified by column chromatography on silica gel (eluent: EtOAc/PE=0˜20%) to afford N,N-bis(2,4-dimethoxybenzyl)-6-(trifluoromethyl)pyridazin-3-amine (2.0 g, 39%) as an off-white solid.

Synthesis of N,N-bis(2,4-dimethoxybenzyl)-5-iodo-6-(trifluoromethyl)pyridazin-3-amine. To a mixture of 2,2,6,6-tetramethylpiperidine (0.15 g, 1.1 mmol) in THF (6 mL) was added nBuLi (0.44 mL, 1.1 mmol) at −78° C. The mixture was stirred at −78° C. for 1 h, then was added a solution of N,N-bis(2,4-dimethoxybenzyl)-6-(trifluoromethyl)pyridazin-3-amine (0.40 g, 0.86 mmol) in THF (5.0 mL) was added and the mixture was stirred at −78° C. for 1 h. Then a solution of I₂ (0.26 g, 1.0 mmol) in THF (4 mL) was added and stirring was continued for another 1 h at −78° C. The reaction mixture was quenched with saturated aq·NH₄Cl (100 mL) and extracted with EtOAc (3×50 mL). The combined organic layers were washed with saturated brine (50 mL), dried over Na₂SO₄ and concentrated in vacuo to get a residue. The residue was purified by column chromatography on silica gel (eluent: EtOAc/PE=0˜20%) to afford N,N-bis(2,4-dimethoxybenzyl)-5-iodo-6-(trifluoromethyl)pyridazin-3-amine (0.35 g, 69%) as a yellow solid.

Synthesis of 5-iodo-6-(trifluoromethyl)pyridazin-3-amine. A mixture of N,N-bis(2,4-dimethoxybenzyl)-5-iodo-6-(trifluoromethyl)pyridazin-3-amine (0.35 g, 0.59 mmol) in TFA (5.0 mL) and DCM (5.0 mL) was stirred at room temperature for 2 h. Then the reaction was quenched with saturated aq·NaHCO₃ (0.10 L) and extracted with EtOAc (3×50 mL). The combined organic layers were washed with saturated brine (50 mL), dried over Na₂SO₄ and concentrated in vacuo to get a residue. The residue was purified by column chromatography on silica gel (eluent: EtOAc/PE=0˜50%) to afford 5-iodo-6-(trifluoromethyl)pyridazin-3-amine (93 mg, 55%) as a light-yellow solid.

Synthesis of (1S,2S)—N-(5-iodo-6-(trifluoromethyl)pyridazin-3-yl)-2-(4-methylpyrimidin-2-yl)cyclopropane-1-carboxamide. To a mixture of 5-iodo-6-(trifluoromethyl)pyridazin-3-amine (50 mg, 0.17 mmol), (1S,2S)-2-(4-methylpyrimidin-2-yl)cyclopropane-1-carboxylic acid (46 mg, 0.26 mmol), and pyridine (0.20 mL) was added T₃P (1.1 g, 50% in EtOAc, 1.7 mmol). After stirring at room temperature for 2 h, the reaction was quenched with water (50 mL) and extracted with EtOAc (3×30 mL). The combined organic layers were washed with saturated brine (30 mL) and concentrated in vacuo. The residue was purified by preparative TLC (eluent: DCM/MeOH=15/1) to afford (1S,2S)—N-(5-iodo-6-(trifluoromethyl)pyridazin-3-yl)-2-(4-methylpyrimidin-2-yl)cyclopropane-1-carboxamide (50 mg, 65%) as an off-white solid.

Synthesis of (1S,2S)—N-(5-(((6-cyclopropyl-8-(3-methyl-2,4-dioxoimidazolidin-1-yl)imidazo[1,2-a]pyridin-2-yl)methyl)amino)-6-(trifluoromethyl)pyridazin-3-yl)-2-(4-methylpyrimidin-2-yl)cyclopropane-1-carboxamide (I-640). A mixture of (1S,2S)—N-(5-iodo-6-(trifluoromethyl)pyridazin-3-yl)-2-(4-methylpyrimidin-2-yl)cyclopropane-1-carboxamide (40 mg, 0.089 mmol), 1-(2-(aminomethyl)-6-cyclopropylimidazo-[1,2-a]pyridin-8-yl)-3-methylimidazolidine-2,4-dione (40 mg, 0.13 mmol), and DIPEA (34 mg, 0.26 mmol) in ⁱPrOH (0.50 mL) was stirred in a sealed tube. After degassing with N₂ for 1 min, the reaction was irradiated in a microwave reactor at 120° C. for 2 h. The reaction mixture was quenched with saturated aq·NaHCO₃ (50 mL) and extracted with EtOAc (3×30 mL). The combined organic layers were washed with saturated brine (30 mL) and concentrated in vacuo. The residue was purified by preparative TLC (eluent: MeOH/DCM=10%) to afford (1S,2S)—N-(5-(((6-cyclopropyl-8-(3-methyl-2,4-dioxoimidazolidin-1-yl)imidazo[1,2-a]pyridin-2-yl)methyl)amino)-6-(trifluoromethyl)pyridazin-3-yl)-2-(4-methylpyrimidin-2-yl)cyclopropane-1-carboxamide (30 mg, 54%) as a white solid. ESI-MS [M+H]+: 621.1, ¹H NMR (400 MHz, DMSO) δ 11.34 (s, 1H), 8.52 (d, J=5.0 Hz, 1H), 8.25 (s, 1H), 7.95 (s, 1H), 7.73 (s, 1H), 7.45-7.39 (m, 2H), 7.20 (d, J=5.0 Hz, 1H), 5.03-4.88 (m, 2H), 4.53 (d, J=5.1 Hz, 2H), 2.99 (s, 3H), 2.69-2.62 (m, 2H), 2.41 (s, 3H), 1.99-1.88 (m, 1H), 1.53 (s, 2H), 0.98-0.90 (m, 2H), 0.68-0.61 (m, 2H).

### Example 641

### Synthesis of (1S,2S)-2-(3-chlorophenyl)-N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)cyclopropane-1-carboxamide (I-641)

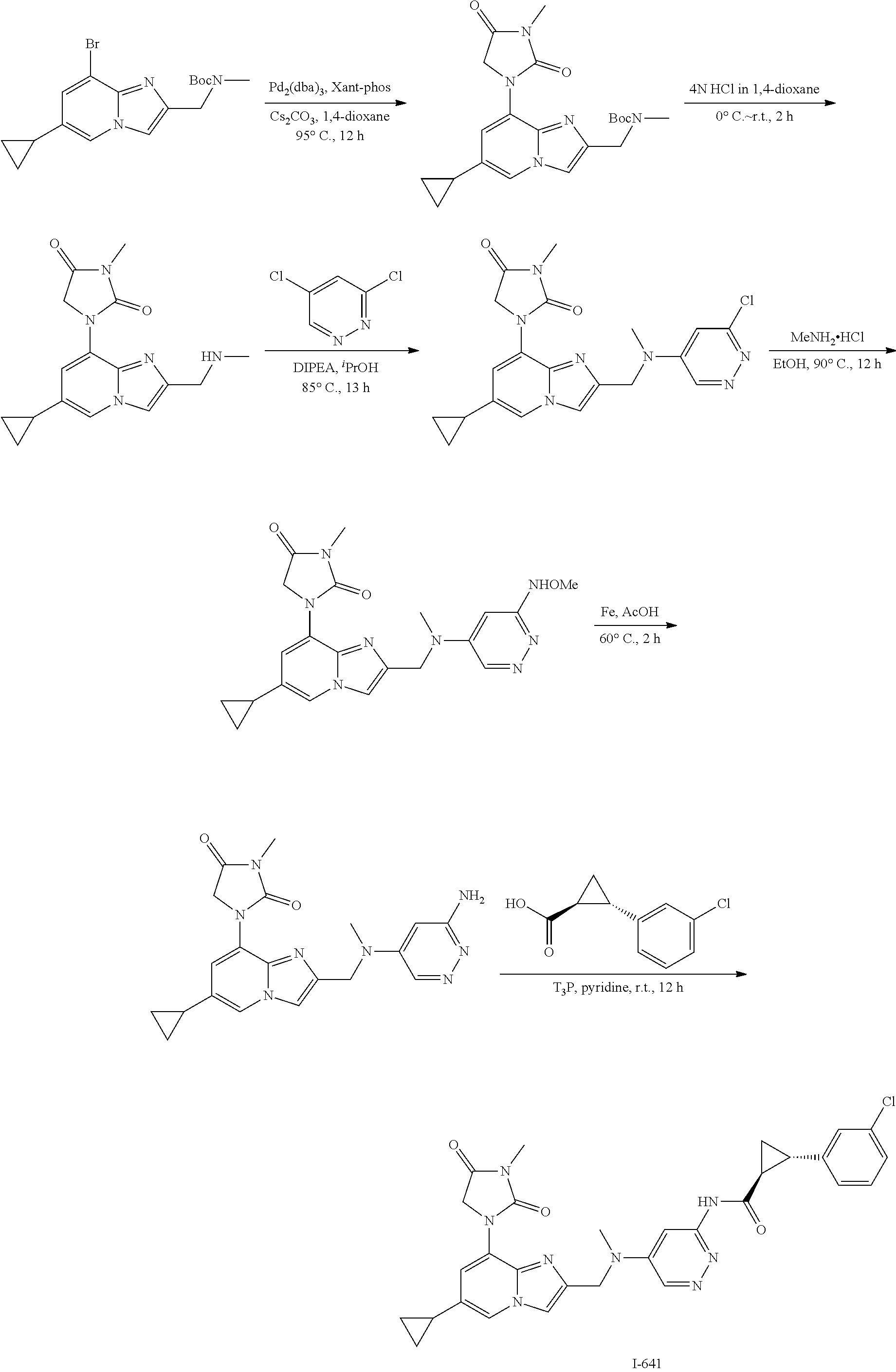

Synthesis of tert-butyl ((6-cyclopropyl-8-(3-methyl-2,4-dioxoimidazolidin-1-yl)imidazo[1,2-a]pyridin-2-yl)methyl)(methyl)carbamate. To a mixture of tert-butyl ((8-bromo-6-cyclopropylimidazo[1,2-a]pyridin-2-yl)methyl)(methyl)carbamate (0.40 g, 1.1 mmol), 3-methylimidazolidine-2,4-dione (0.36 g, 3.2 mmol), and Cs₂CO₃ (1.0 g, 3.1 mmol) in dioxane (20 mL) were added Pd₂(dba)₃ (91 mg, 0.099 mmol) and Xant-phos (58 mg, 0.10 mmol). After stirring at 95° C. for 12 h under N₂, the reaction mixture was filtered through Celite® and the filter cake was washed with DCM/MeOH (10/1, 100 mL). The filtrate was concentrated in vacuo to give the crude, which was purified by column chromatography on silica gel (eluent: PE/EtOAc=10/1˜1/1) to give tert-butyl ((6-cyclopropyl-8-(3-methyl-2,4-dioxoimidazolidin-1-yl)imidazo[1,2-a]pyridin-2-yl)methyl)(methyl)carbamate (350 mg, 77%) as a yellow solid. ESI-MS [M+H]+: 414.2.

Synthesis of 1-(6-cyclopropyl-2-((methylamino)methyl)imidazo[1,2-a]pyridin-8-yl)-3-methylimidazolidine-2,4-dione. To a solution of tert-butyl ((6-cyclopropyl-8-(3-methyl-2,4-dioxoimidazolidin-1-yl)imidazo[1,2-a]pyridin-2-yl)methyl)(methyl)carbamate (0.35 g, 0.85 mmol) in MeOH (10 mL) was added HCl (10 mL, 4N HCl in dioxane, 40 mmol). The resulting mixture was stirred at room temperature for 2 h and concentrated in vacuo to give 1-(6-cyclopropyl-2-((methylamino)methyl) imidazo[1,2-a]pyridin-8-yl)-3-methylimidazolidine-2,4-dione as the hydrochloric acid salt (0.27 g, quant) as a yellow solid. ESI-MS [M+H]+: 314.1.

Synthesis of 1-(2-(((6-chloropyridazin-4-yl)(methyl)amino)methyl)-6-cyclopropylimidazo[1,2-a]pyridin-8-yl)-3-methylimidazolidine-2,4-dione. To a solution of 1-(6-cyclopropyl-2-((methylamino)methyl)imidazo[1,2-a]pyridin-8-yl)-3-methylimidazolidine-2,4-dione (0.27 g, 0.86 mmol) and 3,5-dichloropyridazine (0.25 g, 1.7 mmol) in i-PrOH (10 mL) was added DIPEA (0.55 mg, 4.3 mmol). The mixture was stirred at 85° C. for 13 h and then concentrated in vacuo to give the crude, which was purified with preparative TLC (eluent: DCM/MeOH=12/1) to give 1-(2-(((6-chloropyridazin-4-yl)(methyl)amino) methyl)-6-cyclopropylimidazo[1,2-a]pyridin-8-yl)-3-methylimidazolidine-2,4-dione (0.25 g, 68%) as a white solid. ESI-MS [M+H]+: 426.2.

Synthesis of 1-(6-cyclopropyl-2-(((6-(methoxyamino)pyridazin-4-yl)(methyl)amino)methyl)imidazo[1,2-a]pyridin-8-yl)-3-methylimidazolidine-2,4-dione. To a solution of 1-(2-(((6-chloropyridazin-4-yl)(methyl)amino)methyl)-6-cyclopropylimidazo[1,2-a]pyridin-8-yl)-3-methylimidazolidine-2,4-dione (0.22 g, 0.52 mmol) in EtOH (15 mL) was added MeNH₂·HCl (0.63 g, 9.3 mmol). The mixture was stirred at 90° C. for 12 h and then concentrated in vacuo. The residue was washed with saturated aq·NaHCO₃ (20 mL) and extracted with DCM (3×30 mL). The combined organic layers were washed with saturated brine (50 mL), dried over Na₂SO₄, and concentrated in vacuo to give the crude 1-(6-cyclopropyl-2-(((6-(methoxyamino)pyridazin-4-yl)(methyl)amino)methyl)imidazo[1,2-a]pyridin-8-yl)-3-methylimidazolidine-2,4-dione (0.22 g, 97%) as a yellow solid. ESI-MS [M+H]+: 437.2.

Synthesis of 1-(2-(((6-aminopyridazin-4-yl)(methyl)amino)methyl)-6-cyclopropylimidazo[1,2-a]pyridin-8-yl)-3-methylimidazolidine-2,4-dione. To a solution of 1-(6-cyclopropyl-2-(((6-(methoxyamino)pyridazin-4-yl)(methyl)amino)methyl)imidazo[1,2-a]pyridin-8-yl)-3-methylimidazolidine-2,4-dione (0.22 g, 0.50 mmol) in EtOH (15 mL) were added AcOH (2.0 mL, 20% H₂O, v/v) and Fe powder (0.14 g, 2.5 mmol). After stirring at 60° C. for 2 h, the reaction mixture was filtered through Celite® and the filtrate was concentrated in vacuo. The residue was added saturated aq·NaHCO₃ (10 mL) and extracted with DCM (3×30 mL). The combined organic layers were washed with saturated brine (30 mL), dried over Na₂SO₄, and concentrated in vacuo to give the crude, which was purified by preparative TLC (eluent: DCM/MeOH=10/1) to give 1-(2-(((6-aminopyridazin-4-yl)(methyl)amino)methyl)-6-cyclopropylimidazo [1,2-a]pyridin-8-yl)-3-methylimidazolidine-2,4-dione (0.12 g, 59%) as a white solid. ESI-MS [M+H]+: 407.1.

Synthesis of (1S,2S)-2-(3-chlorophenyl)-N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)cyclopropane-1-carboxamide (I-641). A mixture of 1-(2-(((6-aminopyridazin-4-yl)(methyl)amino)methyl)-6-cyclopropylimidazo[1,2-a]pyridin-8-yl)-3-methylimidazolidine-2,4-dione (50 mg, 0.12 mmol), (1S,2S)-2-(3-chlorophenyl)cyclopropane-1-carboxylic acid (36 mg, 0.18 mg), pyridine (0.25 mL), and T₃P (0.53 g, 1.7 mmol) was stirred at room temperature for 12 h. The reaction mixture was quenched with saturated aq·NaHCO₃ (10 mL) and extracted with DCM (3×20 mL). The combined organic layers were washed with saturated brine (30 mL), dried over Na₂SO₄, concentrated in vacuo to give the crude, which was purified with Pre-TLC (eluent: DCM/MeOH=20/1) to give the product (1S,2S)-2-(3-chlorophenyl)-N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)cyclopropane-1-carboxamide (48 mg, 68%) as a white solid. ESI-MS [M+H]+: 585.2, ¹H NMR (400 MHz, DMSO) δ 10.91 (s, 1H), 8.67 (d, J=2.8 Hz, 1H), 8.22 (d, J=1.0 Hz, 1H), 7.77 (s, 1H), 7.71 (s, 1H), 7.41-7.24 (m, 4H), 7.15 (d, J=7.6 Hz, 1H), 4.88 (s, 2H), 4.72 (s, 2H), 3.13 (s, 3H), 2.96 (s, 3H), 2.71-2.63 (m, 1H), 2.35-2.28 (m, 1H), 2.00-1.88 (m, 1H), 1.52-1.43 (m, 1H), 1.45-1.35 (m, 1H), 1.01-0.87 (m, 2H), 0.70-0.54 (m, 2H)

### Example 642

### Synthesis of (1S,2S)—N-(4-(((6-cyclopropyl-8-(3-methyl-2,4-dioxoimidazolidin-1-yl)imidazo-[1,2-a]pyridin-2-yl)methyl)amino)-6-methylpyridin-2-yl)-2-(4-methylpyrimidin-2-yl)cyclopropane-1-carboxamide (I-642)

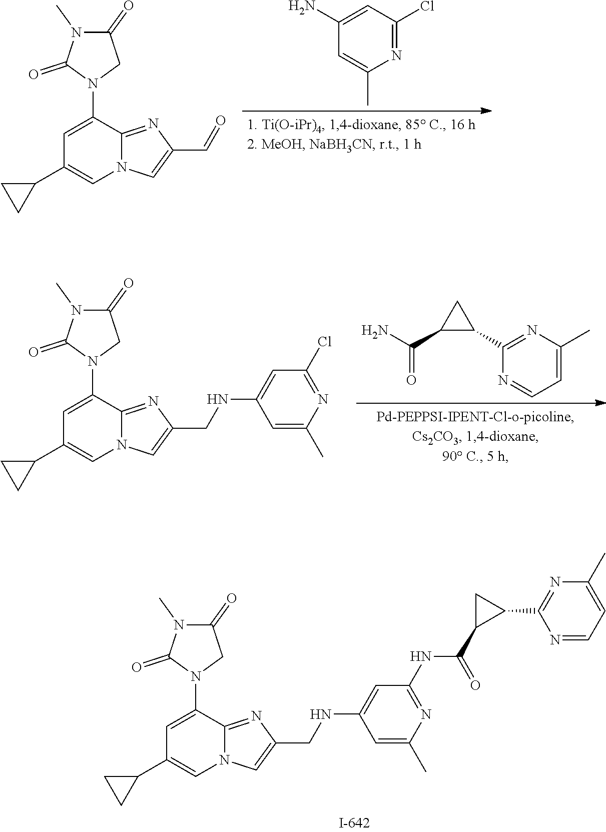

Synthesis of 1-(2-(((2-chloro-6-methylpyridin-4-yl)amino)methyl)-6-cyclopropylimidazo[1,2-a]pyridin-8-yl)-3-methylimidazolidine-2,4-dione. A mixture of 6-cyclopropyl-8-(3-methyl-2,4-dioxoimidazolidin-1-yl)imidazo[1,2-a]pyridine-2-carbaldehyde (149 mg, 0.5 mmol), 2-chloro-6-methylpyridin-4-amine (78.0 mg, 0.55 mmol), and Ti(O-iPr)₄ (710 mg, 2.5 mmol) in 1,4-dioxane (6.0 mL) was stirred at 85° C. for 16 h. Then the reaction mixture was cooled to 0° C. and then added MeOH (3 mL), followed by NaBH₃CN (94.5 mg, 1.5 mmol). The reaction mixture was stirred at room temperature for 1 h. The reaction was quenched with water (10 mL) and extracted with EtOAc (3×20 mL). The combined organic layers were washed with brine (20 mL), dried over anhydrous Na₂SO₄, and concentrated in vacuo to give the crude product, which was purified by preparative TLC (eluent: DCM/MeOH=15/1) to give 1-(2-(((2-chloro-6-methylpyridin-4-yl)amino)methyl)-6-cyclopropylimidazo[1,2-a]pyridin-8-yl)-3-methylimidazolidine-2,4-dione (100 mg, 48%) as a light yellow solid. ESI-MS [M+H]+: 425.2.

Synthesis of (1S,2S)—N-(4-(((6-cyclopropyl-8-(3-methyl-2,4-dioxoimidazolidin-1-yl)imidazo[1,2-a]pyridin-2-yl)methyl)amino)-6-methylpyridin-2-yl)-2-(4-methylpyrimidin-2-yl)cyclopropane-1-carboxamide (I-642). To a mixture of 1-(2-(((2-chloro-6-methylpyridin-4-yl)amino)methyl)-6-cyclopropylimidazo-[1,2-a]pyridin-8-yl)-3-methylimidazolidine-2,4-dione (80 mg, 0.19 mmol), (1S,2S)-2-(4-methylpyrimidin-2-yl)cyclopropane-1-carboxamide (34 mg, 0.19 mmol), and Cs₂CO₃ (185 mg, 0.57 mmol) in 1,4-dioxane (10 mL) was added Pd-PEPPSI-IPENT-Cl-o-picoline (16 mg, 0.02 mmol). After stirring at 90° C. for 5 h under N₂, the reaction mixture was filtered through Celite® and the filter cake was washed with DCM/MeOH (10/1, 20 mL). The filtrate was concentrated in vacuo to give the crude, which was purified by preparative HPLC to give (1S,2S)—N-(4-(((6-cyclopropyl-8-(3-methyl-2,4-dioxoimidazolidin-1-yl)-imidazo[1,2-a]pyridin-2-yl)methyl)amino)-6-methylpyridin-2-yl)-2-(4-methylpyrimidin-2-yl)cyclopropane-1-carboxamide (22 mg, 21%) as a white solid. ESI-MS [M+H]+: 566.3. ¹H NMR (400 MHz, DMSO) δ 10.39 (s, 1H), 8.51 (d, J=5.1 Hz, 1H), 8.26 (d, J=1.1 Hz, 1H), 7.70 (s, 1H), 7.34 (d, J=1.5 Hz, 1H), 7.30 (s, 1H), 7.19 (d, J=5.1 Hz, 1H), 7.01 (t, J=5.9 Hz, 1H), 6.22 (d, J=1.7 Hz, 1H), 4.93 (s, 2H), 4.36 (d, J=5.9 Hz, 2H), 2.98 (s, 3H), 2.54-2.52 (m, 2H), 2.41 (s, 3H), 2.17 (s, 3H), 1.99-1.86 (m, 1H), 1.54-1.39 (m, 2H), 0.99-0.86 (m, 2H), 0.71-0.54 (m, 2H).

### Example 643

### Synthesis of (1S,2S)—N-(3-((6-cyclopropyl-8-(3-methyl-2,4-dioxoimidazolidin-1-yl)imidazo[1,2-a]pyridin-2-yl)methoxy)phenyl)-2-(4-methylpyrimidin-2 l)cyclopropane-1-carboxamide (I-643)

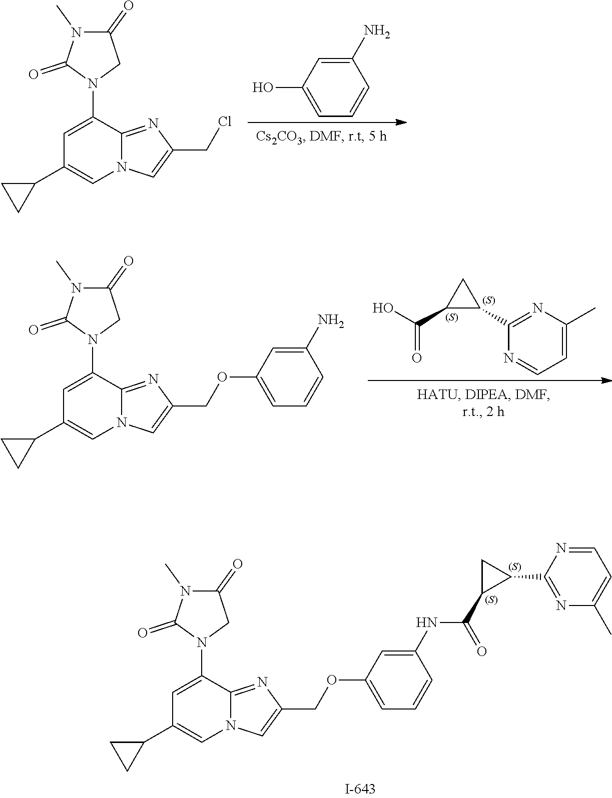

Synthesis of 1-(2-((3-aminophenoxy)methyl)-6-cyclopropylimidazo[1,2-a]pyridin-8-yl)-3-methylimidazolidine-2,4-dione. A mixture of 1-(2-(chloromethyl)-6-cyclopropylimidazo[1,2-a]pyridin-8-yl)-3-methylimidazolidine-2,4-dione (0.38 g, 1.2 mmol), 3-aminophenol (0.14 g, 1.3 mmol), and Cs₂CO₃ (0.78 g, 2.4 mmol) in DMF (10 mL) was stirred at room temperature for 5 h. The reaction mixture was poured into water (100 mL) and extracted with EtOAc (3×40 mL). The combined organics were washed and brine (3×30 mL), dried over Na₂SO₄ then concentrated in vacuo to give the crude product, which was purified by column chromatography on silica gel (eluent: EtOAc/PE from 0 to 60%) to give 1-(2-((3-aminophenoxy)methyl)-6-cyclopropylimidazo[1,2-a]pyridin-8-yl)-3-methylimidazolidine-2,4-dione (0.15 g, 32%) as a yellow solid. ESI-MS [M+H]⁺: 392.1.

Synthesis of (1S,2S)—N-(3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (I-643). To a stirred solution of 1-(2-((3-aminophenoxy)methyl)-6-cyclopropylimidazo[1,2-a]pyridin-8-yl)-3-methylimidazolidine-2,4-dione (50 mg, 0.13 mmol) and (1S,2S)-2-(4-methylpyrimidin-2-yl)cyclopropane-1-carboxylic acid (23 mg, 0.13 mmol) in DMF (3.0 mL) was added HATU (58 mg, 015 mmol) and DIPEA (50 mg, 0.38 mmol). The resulting mixture was stirred at room temperature for 2 h. The reaction mixture was poured into water (30 mL) and extracted with EtOAc (3×30 mL). The combined organics were washed with brine (2×30 mL), dried over Na₂SO₄, concentrated in vacuo to give the crude product, which was purified by column chromatography (eluent: EtOAc/PE from 0 to 60%) to give (1S,2S)—N-(3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (45 mg, 53%) as a white solid. ESI-MS [M+H]⁺: 552.2. ¹H NMR (400 MHz, DMSO) δ 10.32 (s, 1H), 8.53 (d, J=5.1 Hz, 1H), 8.29 (d, J=1.1 Hz, 1H), 7.93 (s, 1H), 7.43-7.40 (m, 1H), 7.38 (d, J=1.4 Hz, 1H), 7.23-7.16 (m, 2H), 7.15-7.09 (m, 1H), 6.75 (dd, J=8.1, 1.8 Hz, 1H), 5.15 (s, 2H), 4.90 (s, 2H), 2.97 (s, 3H), 2.57-2.52 (m, 1H), 2.45-2.38 (m, 1H), 2.42 (s, 3H), 1.99-1.93 (m, 1H), 1.56-1.45 (m, 2H), 1.00-0.91 (m, 2H), 0.71-0.62 (m, 2H).

### Example 644

### Synthesis of (1S,2S)—N-(3-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (I-644)

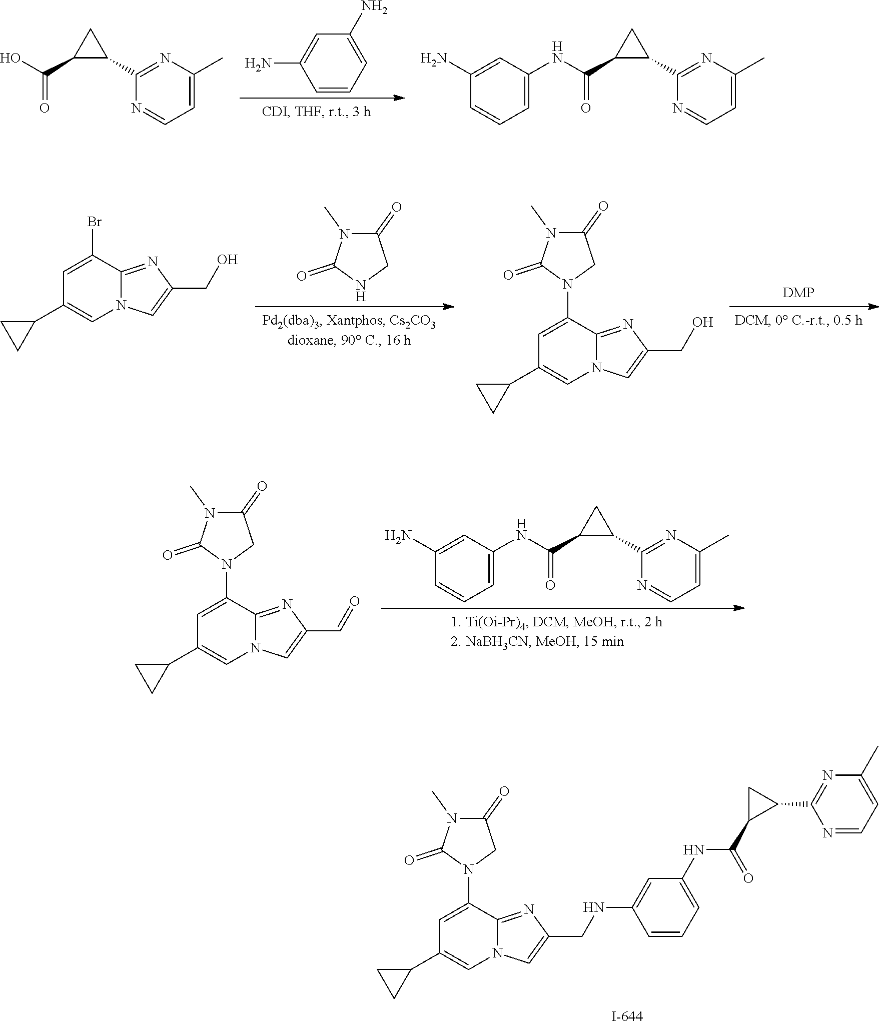

Synthesis of (1S,2S)—N-(3-aminophenyl)-2-(4-methylpyrimidin-2-yl) cyclopropane-1-carboxamide. To a mixture of (1S,2S)-2-(4-methylpyrimidin-2-yl)cyclopropane-1-carboxylic acid (200 mg, 1.29 mmol) in dry THF (10 mL) was added benzene-1,3-diamine (266.4 mg, 2.464 mmol). The mixture was stirred at room temperature for 45 min and then was added CDI in dry THF (2 mL). After stirring at room temperature for 2 h, the reaction was quenched with water (30 mL) and extracted with EtOAc (3×30 mL). The combined organic layers were washed with saturated brine (30 mL), dried over anhydrous Na₂SO₄, and concentrated in vacuo. The residue was purified by column chromatography (eluent: EtOAc/PE=0˜40%) to afford (1S,2S)—N-(3-aminophenyl)-2-(4-methylpyrimidin-2-yl)cyclopropane-1-carboxamide (216 mg, 81%) as brown solid. ESI-MS [M+H]+: 179.1.

Synthesis of (1S,2S)—N-(3-aminophenyl)-2-(4-methylpyrimidin-2-yl)cyclopropane-1-carboxamide. To a mixture of (8-bromo-6-cyclopropylimidazo[1,2-a]pyridin-2-yl)methanol (300 mg, 1.12 mmol), 3-methylimidazolidine-2,4-dione (383.4 mg, 3.36 mmol), and Cs₂CO₃ (912.8 mg, 2.8 mmol) in 1,4-dioxane (25 mL) were added Pd₂(dba)₃ (207.4 mg, 0.22 mmol) and Xantphos (259 mg, 0.45 mmol). After stirring at 90° C. for 16 h under N₂, the reaction mixture was filtered through Celite® and the filter cake was washed with DCM/MeOH (10/1, 50 mL). The filtrate was concentrated in vacuo to give the crude, which was purified by silica gel column chromatography (eluent:MeOH/DCM=0˜5%) to afford 1-(6-cyclopropyl-2-(hydroxymethyl)imidazo[1,2-a]pyridin-8-yl)-3-methylimidazolidine-2,4-dione (142 mg, 65%) as pale solid. ESI-MS [M+H]+: 301.1.

Synthesis of 6-cyclopropyl-8-(3-methyl-2,4-dioxoimidazolidin-1-yl)imidazo[1,2-a]pyridine-2-carbaldehyde. To a mixture of 1-(6-cyclopropyl-2-(hydroxymethyl)imidazo[1,2-a]pyridin-8-yl)-3-methylimidazolidine-2,4-dione (140 mg, 0.47 mmol) in DCM (3.6 mL) was added Dess Martin periodinane (493 mg, 1.16 mmol). The reaction mixture was stirred at room temperature for 0.5 h. The resulting solution was washed with saturated aq·NaHCO₃ (20 mL) and saturated aq·Na₂S₂O₃ (20 mL) and then extracted with EtOAc (3×30 mL). The combined organic layers were washed with brine (30 mL), dried over Na₂SO₄ and concentrated in vacuo. The residue was purified by silica gel column chromatography (eluent: MeOH/DCM=0˜5%) to afford 6-cyclopropyl-8-(3-methyl-2,4-dioxoimidazolidin-1-yl)imidazo[1,2-a]pyridine-2-carbaldehyde (67 mg, 47%) as pale solid. ESI-MS [M+H]+: 299.1.

Synthesis of (1S,2S)—N-(3-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (I-644). To a mixture of (1S,2S)—N-(3-aminophenyl)-2-(4-methylpyrimidin-2-yl)cyclopropane-1-carboxamide (34.7 mg, 0.13 mmol) in DCM (1 mL) and MeOH (1 mL) was added 6-cyclopropyl-8-(3-methyl-2,4-dioxoimidazolidin-1-yl)imidazo[1,2-a]pyridine-2-carbaldehyde (35 mg, 0.12 mol) and Ti (Oi-Pr)₄ (168 mg, 0.59 mmol). The mixture was stirred at 50° C. for 2 h, then NaBH₃CN (62.84 mg, 0.413 mmol) was added. After stirring at room temperature for 15 min, the reaction was quenched with water (20 mL) and extracted with EtOAc (3×30 mL). The combined organic phases were washed with brine (30 mL), dried over anhydrous Na₂SO₄ and concentrated in vacuo to give the crude product, which was purified by pre-TLC (eluent: DCM:MeOH=20:1) to give (1S,2S)—N-(3-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (27.2 mg, 77%) as pale solid. ESI-MS [M+H]+: 551.2. ¹H NMR (400 MHz, DMSO) δ 10.04 (s, 1H), 8.52 (d, J=5.1 Hz, 1H), 8.24 (d, J=1.1 Hz, 1H), 7.67 (s, 1H), 7.32 (d, J=1.4 Hz, 1H), 7.20 (d, J=5.1 Hz, 1H), 6.97-6.92 (m, 2H), 6.78 (d, J=8.0 Hz, 1H), 6.33 (d, J=7.8 Hz, 1H), 6.19 (t, J=6.0 Hz, 1H), 4.91 (s, 2H), 4.30 (d, J=5.8 Hz, 2H), 2.97 (s, 3H), 2.47-2.45 (m, 1H), 2.41-2.37 (m, 4H), 1.96-1.89 (m, 1H), 1.50-1.43 (m, 2H), 0.95-0.90 (m, 2H), 0.66-0.62 (m, 2H).

### Examples 645-646

### Synthesis of (1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide (I-645) and (1S,2S)—N-(3-(((6-cyclopropyl-8-(3-methyl-2,4-dioxoimidazolidin-1-yl)imidazo[1,2-a]pyridin-2-yl)methyl)sulfonyl)phenyl)-2-(4-methylpyrimidin-2-yl)cyclopropane-1-carboxamide (I-646)

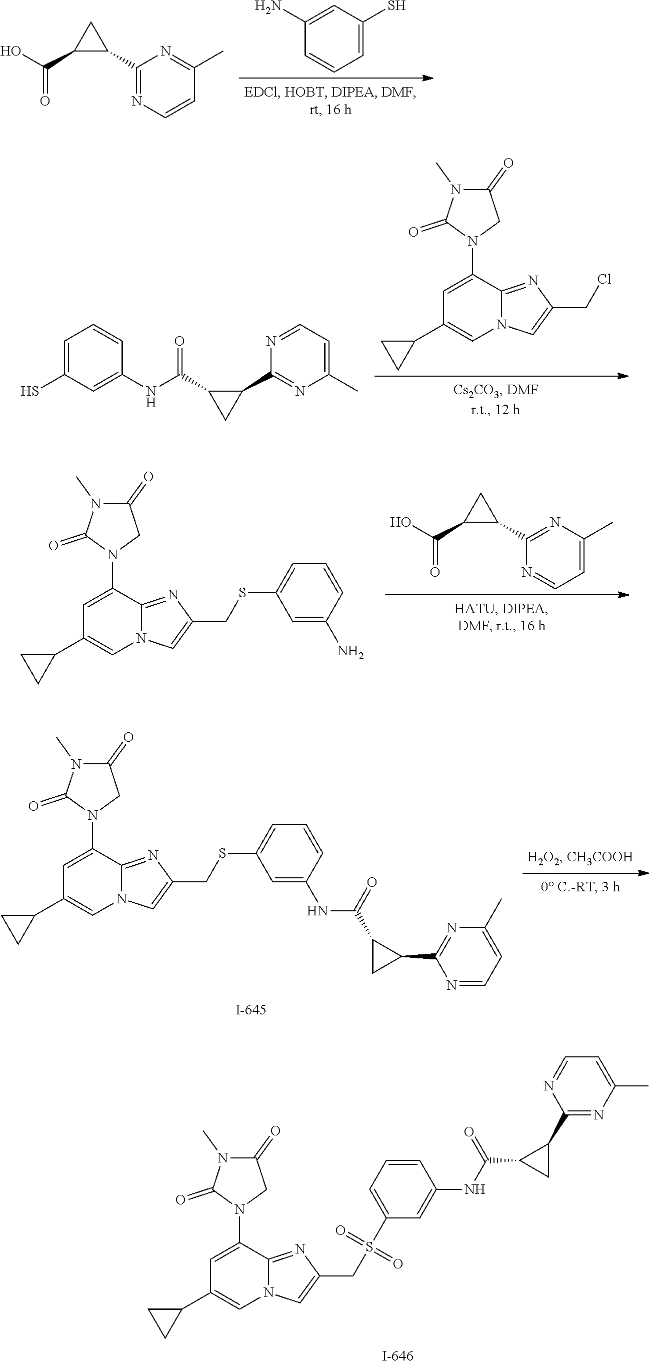

Synthesis of (1S,2S)—N-(3-mercaptophenyl)-2-(4-methylpyrimidin-2-yl)cyclopropane-1-carboxamide. To a mixture of (1S,2S)-2-(4-methylpyrimidin-2-yl)cyclopropane-1-carboxylic acid (200 mg, 1.12 mmol) in DMF (4 mL) was added 3-aminobenzenethiol (421 mg, 3.36 mmol), EDCI (428 mg, 2.24 mmol), HOBt (303 mg, 2.24 mmol), and DIPEA (1.08 g, 8.4 mmol), and the mixture was stirred at room temperature for 16 h. The reaction mixture was quenched with water (50 mL) and extracted with EtOAc (30 mL×3). The combined organic layers were washed with brine (30 mL), dried over anhydrous Na2SO4, and concentrated in vacuo to give the crude, which was purified by column chromatography (eluent: EtOAc/PE=0-10%) to give (1S,2S)—N-(3-mercaptophenyl)-2-(4-methylpyrimidin-2-yl)cyclopropane-1-carboxamide (150 mg, yield 47%) as a yellow oil. ESI-MS [M+H]+: 286.1

Synthesis of 1-(2-(((3-aminophenyl)thio)methyl)-6-cyclopropylimidazo[1,2-a]pyridin-8-yl)-3-methylimidazolidine-2,4-dione. To a mixture of (1S,2S)—N-(3-mercaptophenyl)-2-(4-methylpyrimidin-2-yl)cyclopropane-1-carboxamide (100 mg, 0.35 mmol) in DMF (5 mL) was added 1-(2-(chloromethyl)-6-cyclopropylimidazo[1,2-a]pyridin-8-yl)-3-methylimidazolidine-2,4-dione (112 mg, 0.35 mmol) and CsCO3 (342 mg, 1.05 mmol), and the mixture was stirred at room temperature for 12 h. The reaction mixture was quenched with water (50 mL) and extracted with EtOAc (30 mL×3). The combined organic layers were washed with brine (30 mL), dried over anhydrous Na₂SO₄, and concentrated in vacuo to give the crude, which was purified by column chromatography (eluent: MeOH/DCM=0-8%) to give 1-(2-(((3-aminophenyl)thio)methyl)-6-cyclopropylimidazo[1,2-a]pyridin-8-yl)-3-methylimidazolidine-2,4-dione (50 mg, 35%) as a yellow oil. ESI-MS [M+H]+: 408.1

Synthesis of (1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide (I-645). A mixture of 1-(2-(((3-aminophenyl)thio)methyl)-6-cyclopropylimidazo[1,2-a]pyridin-8-yl)-3-methylimidazolidine-2,4-dione (50 mg, 0.1 mmol), (1S,2S)-2-(4-methylpyrimidin-2-yl)cyclopropane-1-carboxylic acid (45 mg, 0.2 mmol), HATU (114 mg, 0.3 mmol), and DIPEA (39 mg, 0.3 mmol) in DMF (2 mL) was stirred at room temperature for 16 h under N₂. The reaction mixture was quenched with water (50 mL) and extracted with EtOAc (3×50 mL). The combined organics were washed with brine (50 mL), dried over Na₂SO₄ and concentrated in vacuo. The residue was purified by flash chromatography (eluent: EtOAc/PE=0-20%) to give (1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide (60 mg, yield 60%) as a colorless oily liquid ESI-MS: [M+H]+, 568.1. ¹H NMR (400 MHz, DMSO) δ 10.35 (s, 1H), 8.53 (d, J=5.1 Hz, 1H), 8.23 (d, J=1.0 Hz, 1H), 7.77 (s, 1H), 7.70 (s, 1H), 7.37-7.33 (m, 2H), 7.23-7.19 (m, 2H), 7.08 (d, J=8.3 Hz, 1H), 4.86 (s, 2H), 4.28 (s, 2H), 2.96 (s, 3H), 2.56-2.54 (m, 1H), 2.46-2.45 (m, 1H), 2.42 (s, 3H), 1.95-1.91 (m, 1H), 1.53-1.43 (m, 2H), 0.96-0.91 (m, 2H), 0.66-0.62 (m, 2H).

Synthesis of (1S,2S)—N-(3-(((6-cyclopropyl-8-(3-methyl-2,4-dioxoimidazolidin-1-yl)imidazo[1,2-a]pyridin-2-yl)methyl)sulfonyl)phenyl)-2-(4-methylpyrimidin-2-yl)cyclopropane-1-carboxamide (I-646). To a mixture of (1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide (60 mg, 0.11 mmol) in AcOH (5 mL) was added H₂O₂ (0.08 mL, 0.11 mmol) and the mixture was stirred for 3 h at room temperature under N₂. The reaction mixture was concentrated in vacuo and the residue was purified by preparative TLC (eluent: DCM/MeOH=10/1) to afford (1S,2S)—N-(3-(((6-cyclopropyl-8-(3-methyl-2,4-dioxoimidazolidin-1-yl)imidazo[1,2-a]pyridin-2-yl)methyl)sulfonyl)phenyl)-2-(4-methylpyrimidin-2-yl)cyclopropane-1-carboxamide (27 mg, 41%) as white solid. ESI-MS: [M+H]+, 600.1. ¹H NMR (400 MHz, DMSO) δ 10.68 (s, 1H), 8.53 (d, J=5.1 Hz, 1H), 8.29 (s, 1H), 8.06 (s, 1H), 7.84 (s, 1H), 7.76 (d, J=7.7 Hz, 1H), 7.53-7.45 (m, 2H), 7.34 (d, J=1.3 Hz, 1H), 7.21 (d, J=5.1 Hz, 1H), 4.76 (s, 2H), 4.52 (s, 2H), 2.93 (s, 3H), 2.55-2.54 (m, 1H), 2.46-2.45 (m, 1H), 2.42 (s, 3H), 1.97-1.92 (m, 1H), 1.51-1.47 (m, 2H), 0.97-0.92 (m, 2H), 0.67-0.64 (m, 2H).

### Example 647

### Synthesis of (1S,2S)-2-(3-chlorophenyl)-N-(6-(((6-cyclopropyl-8-(2-oxopiperazin-1-yl)imidazo[1,2-a]pyridin-2-yl)methyl)amino)pyrimidin-4-yl)cyclopropane-1-carboxamide (I-647)

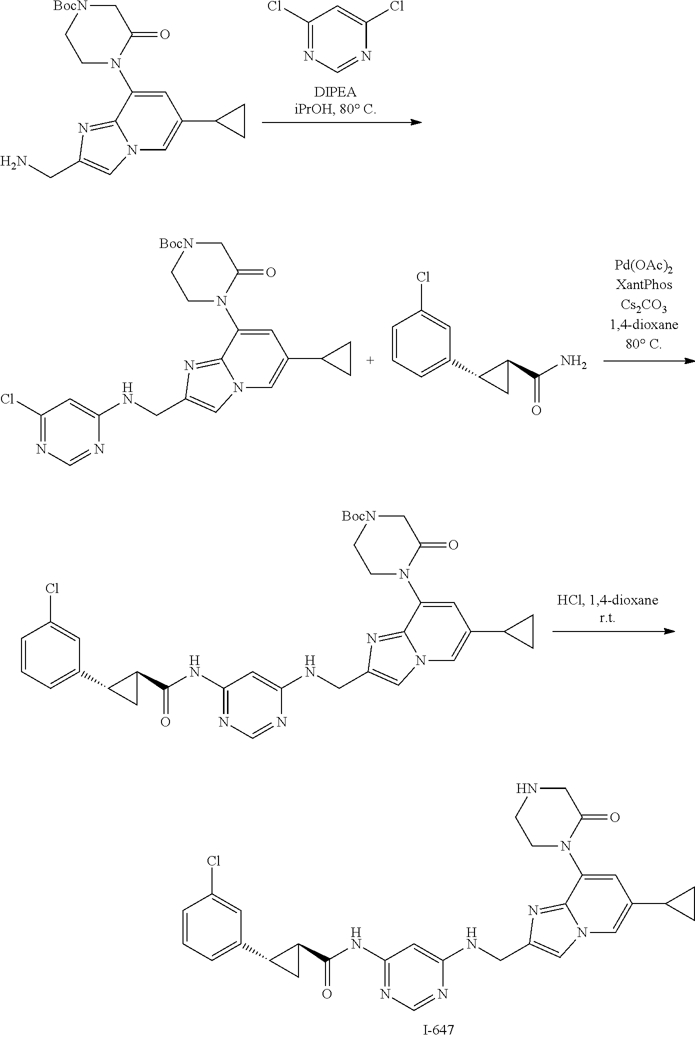

Synthesis of tert-butyl 4-(2-(((6-chloropyrimidin-4-yl)amino)methyl)-6-cyclopropylimidazo[1,2-a]pyridin-8-yl)-3-oxopiperazine-1-carboxylate. A mixture of tert-butyl 4-(2-(aminomethyl)-6-cyclopropylimidazo[1,2-a]pyridin-8-yl)-3-oxopiperazine-1-carboxylate (0.13 g, 0.32 mmol), 4,6-dichloropyrimidine (0.048 g, 0.32 mmol), and DIPEA (0.56 mL, 3.2 mmol) in iPrOH (1.8 mL) was stirred at 80° C. for 18 h. Further 4,6-dichloropyrimidine (0.048 g, 0.32 mmol) was added and the mixture was stirred at 80° C. for 1 h. The mixture was concentrated in vacuo and purified by column chromatography on silica gel, eluting with 0-20% (7 N NH₃ in MeOH) in DCM to give the title compound (0.14 g, 85%) as a yellow gum. ESI-MS (M+H)+: 498.

Synthesis of tert-butyl 4-(2-(((6-((1S,2S)-2-(3-chlorophenyl)cyclopropane-1-carbox amido)pyrimidin-4-yl)amino)methyl)-6-cyclopropylimidazo[1,2-a]pyridin-8-yl)-3-oxopiperazine-1-carboxylate. A mixture of tert-butyl 4-(2-(((6-chloropyrimidin-4-yl)amino)methyl)-6-cyclopropylimidazo[1,2-a]pyridin-8-yl)-3-oxopiperazine-1-carboxylate (0.14 g, 0.28 mmol), (1S,2S)-2-(3-chlorophenyl)cyclopropane-1-carboxamide (0.075 g, 0.39 mmol), Pd(OAc)₂ (0.012 g, 0.055 mmol), XantPhos (0.064 g, 0.11 mmol), and Cs₂CO₃ (0.27 g, 0.83 mmol) in 1,4-dioxane (3.0 mL) was degassed with nitrogen and stirred at 80° C. for 18 h. Further Pd(OAc)₂ (0.012 g, 0.055 mmol), XantPhos (0.064 g, 0.11 mmol), and 1,4-dioxane (1.0 mL) were added and the mixture was degassed with nitrogen. The mixture was stirred at 100° C. for 2 h, then concentrated in vacuo. The residue was purified by column chromatography on silica gel (25 g), eluting with 0-20% (7 N NH₃ in MeOH) in DCM to give the title compound (0.057 g, 31%) as a brown gum, which was used directly in the next step without further purification. ESI-MS (M+H)+: 657.

Synthesis of (1S,2S)-2-(3-chlorophenyl)-N-(6-(((6-cyclopropyl-8-(2-oxopiperazin-1-yl)imidazo[1,2-a]pyridin-2-yl)methyl)amino)pyrimidin-4-yl)cyclopropane-1-carboxamide (I-647). HCl (4.0 M in 1,4-dioxane, 1.0 mL, 4.0 mmol) was added to a solution of tert-butyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)-3-oxopiperazine-1-carboxylate (0.054 g, 0.082 mmol) in 1,4-dioxane (3.0 mL). The mixture was stirred at room temperature for 3.5 h then concentrated in vacuo. The residue was loaded onto an SCX cartridge and washed with a mixture of methanol and DCM then with MeOH. The product was eluted with 7 N NH₃ in MeOH and concentrated in vacuo. The residue was purified by preparative HPLC to give the title compound (0.0094 g, 20%) as an off-white solid. ESI-MS (M+H)+: 557.7, ¹H NMR (400 MHz, DMSO) δ 10.63 (s, 1H), 8.32 (s, 1H), 8.21 (s, 1H), 7.93 (s, 1H), 7.67 (s, 1H), 7.36-7.31 (m, 1H), 7.28 (d, J=6.4 Hz, 3H), 7.16 (d, J=7.7 Hz, 1H), 6.95 (s, 1H), 4.58-4.58 (m, 2H), 3.79 (dd, J=5.1, 5.1 Hz, 2H), 3.58 (s, 2H), 3.19 (dd, J=5.1, 5.1 Hz, 2H), 2.45-2.34 (m, 2H), 1.98-1.90 (m, 1H), 1.51-1.39 (m, 2H), 0.97-0.91 (m, 2H), 0.69-0.64 (m, 2H). One NH proton not visible.

### Example 648

### Synthesis of (1S,2S)—N-(6-((6-cyclopropyl-8-(3-methyl-2,4-dioxoimidazolidin-1-yl)imidazo[1,2-a]pyridin-2-yl)methoxy)pyridazin-4-yl)-2-(4-methylpyrimidin-2-yl)cyclopropane-1-carboxamide (I-648)

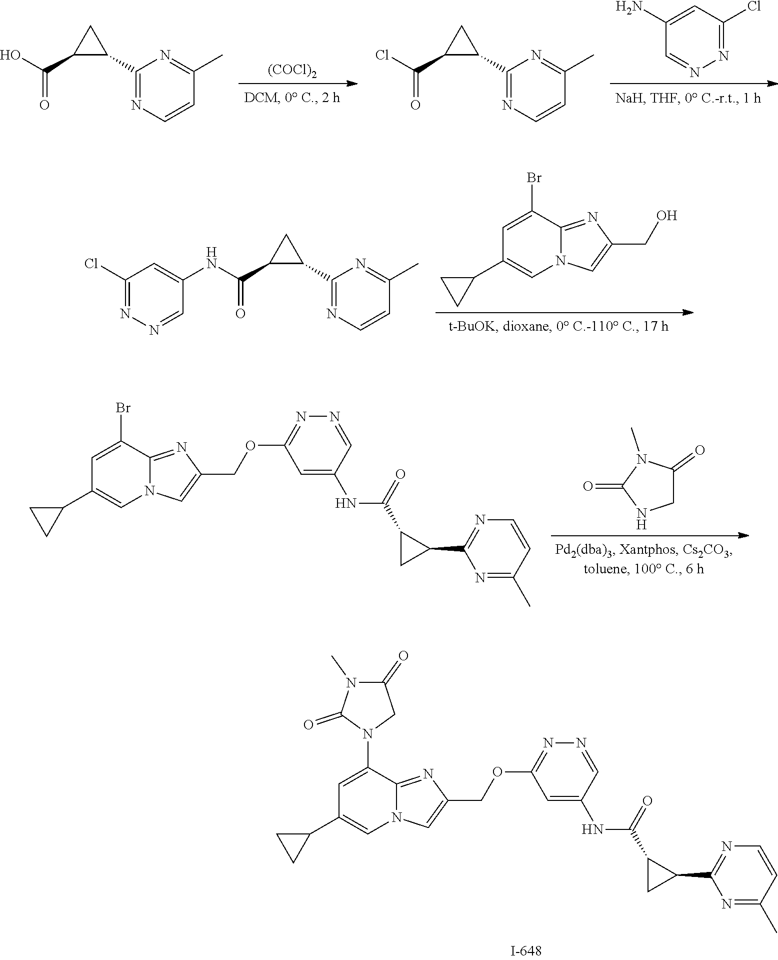

Synthesis of (1S,2S)-2-(4-methylpyrimidin-2-yl)cyclopropane-1-carbonyl chloride. To a solution of (1S,2S)-2-(4-methylpyrimidin-2-yl)cyclopropane-1-carboxylic acid (1.0 g, 5.62 mol) in DCM (20 mL) was added (COCl)₂ (1 mL) at 0° C., and the mixture was stirred at 0° C. for 2 h. The mixture was concentrated in vacuo to give (1S,2S)-2-(4-methylpyrimidin-2-yl)cyclopropane-1-carbonyl chloride (1.1 g, 99%, crude) as a yellow solid which was used directly in the next step. ESI-MS [M+H−35+31]⁺: 193.0.

Synthesis of (1S,2S)—N-(6-chloropyridazin-4-yl)-2-(4-methylpyrimidin-2-yl)cyclopropane-1-carboxamide. To a solution of 6-chloropyridazin-4-amine (1.1 g, 8.42 mmol) in THF (15 mL) was added NaH (1.1 g, 28.0 mmol), and the mixture was stirred at 0° C. for 30 min. Then was added (1S,2S)-2-(4-methylpyrimidin-2-yl)cyclopropane-1-carbonyl chloride (1.1 g, 5.61 mmol) and then the mixture was stirred at 25° C. for 30 min. The reaction was quenched by saturated aq·NH4Cl (100 mL) and extracted with EtOAc (3×50 mL). The combined organic layers were washed with brine (50 mL), dried over Na₂SO₄ and concentrated in vacuo. The residue was purified by silica gel column chromatography (eluent: DCM:MeOH=0˜20:1) to give (1S,2S)—N-(6-chloropyridazin-4-yl)-2-(4-methylpyrimidin-2-yl)cyclopropane-1-carboxamide (560 mg, 35%) as yellow solid. ESI-MS [M+H]⁺: 290.1

Synthesis of (1S,2S)—N-(6-((8-bromo-6-cyclopropylimidazo[1,2-a]pyridin-2-yl)methoxy)pyridazin-4-yl)-2-(4-methylpyrimidin-2-yl)cyclopropane-1-carboxamide. To a mixture of t-BuOK (651 mg, 5.81 mmol) in 1,4-dioxane (20 mL) was added (8-bromo-6-cyclopropylimidazo[1,2-a]pyridin-2-yl)methanol (928 mg, 3.49 mmol) at 0° C., and the mixture was stirred at room temperature for 1 h. (1S,2S)—N-(6-chloropyridazin-4-yl)-2-(4-methylpyrimidin-2-yl)cyclopropane-1-carboxamide (560 mg, 1.94 mmol) was added and then stirred at 110° C. for 16 h. After cooling to room temperature, the mixture was quenched by ice water and extracted with EtOAc (3×50 mL). The combined organic layers were washed with brine (50 mL), dried over Na₂SO₄ and concentrated in vacuo. The residue was purified by silica gel column chromatography (eluent: DCM:MeOH=10:1) to give (1S,2S)—N-(6-((8-bromo-6-cyclopropylimidazo[1,2-a]pyridin-2-yl)methoxy)pyridazin-4-yl)-2-(4-methylpyrimidin-2-yl)cyclopropane-1-carboxamide (205 mg, yield 20%) as yellow solid. ESI-MS [M+H]⁺: 520.1

Synthesis of (1S,2S)—N-(6-((6-cyclopropyl-8-(3-methyl-2,4-dioxoimidazolidin-1-yl)imidazo[1,2-a]pyridin-2-yl)methoxy)pyridazin-4-yl)-2-(4-methylpyrimidin-2-yl)cyclopropane-1-carboxamide (I-648). To a solution of (1S,2S)—N-(6-((8-bromo-6-cyclopropylimidazo[1,2-a]pyridin-2-yl)methoxy)pyridazin-4-yl)-2-(4-methylpyrimidin-2-yl)cyclopropane-1-carboxamide (100 mg, 0.19 mmol) in toluene (20 mL) was added 3-methylimidazolidine-2,4-dione (110 mg, 0.96 mmol), Pd₂(dba)₃ (35 mg, 0.039 mmol), Cs₂CO₃ (188 mg, 0.58 mmol), and Xantphos (45 mg, 0.077 mmol), and the mixture was stirred at 100° C. for 6 h under N₂. The mixture was quenched with water (50 mL) and extracted with EtOAc (3×50 mL). The combined organic layers were washed with brine (50 mL), dried over Na₂SO₄ and concentrated in vacuo. The residue was purified by preparative HPLC to give (1S,2S)—N-(6-((6-cyclopropyl-8-(3-methyl-2,4-dioxoimidazolidin-1-yl)imidazo[1,2-a]pyridin-2-yl)methoxy)pyridazin-4-yl)-2-(4-methylpyrimidin-2-yl)cyclopropane-1-carboxamide (15 mg, 14%) as a white solid. ESI-MS [M+H]+: 554.2. ¹H NMR (400 MHz, DMSO) δ 11.05 (s, 1H), 8.89 (d, J=2.0 Hz, 1H), 8.54 (d, J=5.1 Hz, 1H), 8.31-8.26 (m, 1H), 7.97 (s, 1H), 7.49 (d, J=2.0 Hz, 1H), 7.38 (d, J=1.5 Hz, 1H), 7.23 (d, J=5.1 Hz, 1H), 5.56 (s, 2H), 4.89 (s, 2H), 2.97 (s, 3H), 2.60-2.55 (m, 2H), 2.42 (s, 3H), 2.00-1.92 (m, 1H), 1.63-1.53 (m, 2H), 0.98-0.93 (m, 2H), 0.69-0.64 (m, 2H).

### Example 649

### Synthesis of (1S,2S)—N-(2-((6-cyclopropyl-8-(3-methyl-2,4-dioxoimidazolidin-1-yl)imidazo[1,2-a]pyridin-2-yl)methyl)pyrazolo[1,5-a]pyrazin-4-yl)-2-(4-methylpyrimidin-2-yl)cyclopropane-1-carboxamide (I-649)

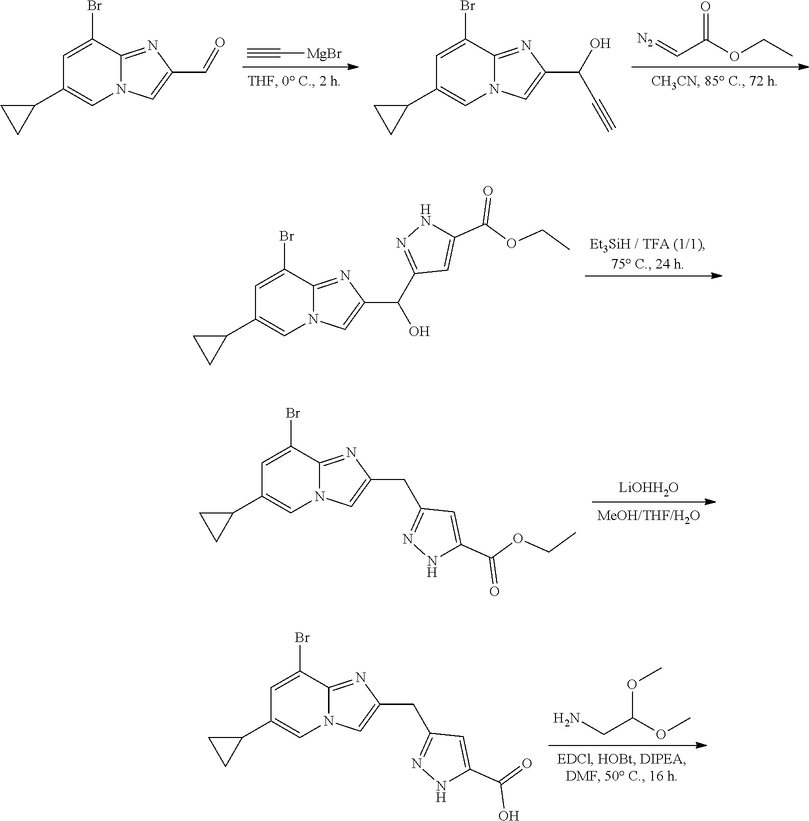

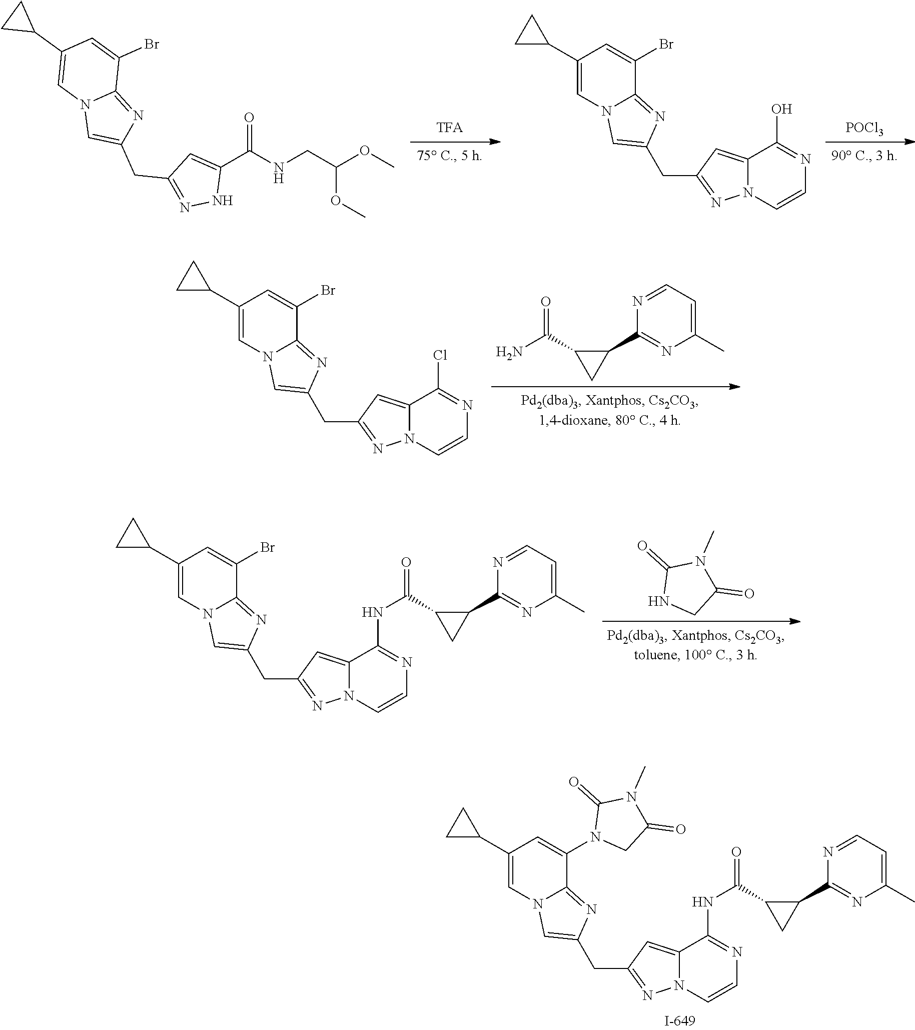

Synthesis of 1-(8-bromo-6-cyclopropylimidazo[1,2-a]pyridin-2-yl)prop-2-yn-1-ol. To a mixture of 8-bromo-6-cyclopropylimidazo[1,2-a]pyridine-2-carbaldehyde (1.0 g, 3.78 mmol) in THF (30.0 mL) was added ethynylmagnesium bromide (22.8 mL, 11.4 mmol, 0.5 M in THF) at 0° C. and the mixture was stirred at 0° C. for 2 h under N₂. The reaction was quenched with saturated aq·NH₄Cl (50 mL) and extracted with EtOAc (3×50 mL). The combined organic layers were washed with brine (50 mL), dried over anhydrous Na₂SO₄, and concentrated in vacuo to give 1-(8-bromo-6-cyclopropylimidazo[1,2-a]pyridin-2-yl)prop-2-yn-1-ol (1.1 g, crude) as a yellow solid which was used for the next step directly. ESI-MS [M+H]⁺: 291.2.

Synthesis of ethyl 3-((8-bromo-6-cyclopropylimidazo[1,2-a]pyridin-2-yl)(hydroxy)methyl)-1H-pyrazole-5-carboxylate. A mixture of 1-(8-bromo-6-cyclopropylimidazo[1,2-a]pyridin-2-yl)prop-2-yn-1-ol (1.0 g, 3.45 mmol) and ethyl 2-diazoacetate (1.18 g, 10.34 mmol) in CH₃CN (20 mL) was stirred at 85° C. for 72 h under N₂. The reaction mixture was quenched with water (50 mL) and extracted with EtOAc (3×50 mL). The combined organic layers were washed with brine (50 mL), dried over anhydrous Na₂SO₄ and concentrated in vacuo. The residue was purified by column chromatography (eluent: DCM/MeOH=30/1) to give ethyl3-((8-bromo-6-cyclopropylimidazo[1,2-a]-pyridin-2-yl)(hydroxy)methyl)-1H-pyrazole-5-carboxylate (700 mg, 50%) as a yellow solid. ESI-MS [M+H]⁺: 405.2.

Synthesis of ethyl 3-((8-bromo-6-cyclopropylimidazo[1,2-a]pyridin-2-yl)methyl)-1H-pyrazole-5-carboxylate. A mixture of ethyl 3-((8-bromo-6-cyclopropylimidazo[1,2-a]pyridin-2-yl)(hydroxy)methyl)-1H-pyrazole-5-carboxylate (300 mg, 0.74) in Et₃SiH/TFA (5 mL/5 mL) was stirred at 75° C. for 24 h. The reaction was quenched with saturated aq·NaHCO₃ (100 mL) and extracted with EtOAc (3×50 mL). The combined organic layers were washed with brine (50 mL), dried over anhydrous Na₂SO₄ and concentrated in vacuo. The residue was purified by column chromatography (eluent: DCM/MeOH=20/1) to give ethyl 3-((8-bromo-6-cyclopropylimidazo[1,2-a]-pyridin-2-yl)methyl)-1H-pyrazole-5-carboxylate (190 mg, 66%) as a yellow oil. ESI-MS [M+H]⁺: 389.2.

Synthesis of 3-((8-bromo-6-cyclopropylimidazo[1,2-a]pyridin-2-yl)methyl)-1H-pyrazole-5-carboxylic acid. A mixture of ethyl 3-((8-bromo-6-cyclopropylimidazo[1,2-a]pyridin-2-yl)methyl)-1H-pyrazole-5-carboxylate (190 mg, 0.5 mmol) in a solution of MeOH/THF/H2O (5 mL/5 mL/1 mL) was stirred at 60° C. for 5 h. The mixture was acidified to pH=3 with HCl (1N) and concentrated in vacuo to give 3-((8-bromo-6-cyclopropylimidazo[1,2-a]pyridin-2-yl)methyl)-1H-pyrazole-5-carboxylic acid (200 mg, crude) as a yellow solid which was used for the next step directly. ESI-MS [M+H]⁺: 361.2.

Synthesis of 3-((8-bromo-6-cyclopropylimidazo[1,2-a]pyridin-2-yl)methyl)-N-(2,2-dimethoxyethyl)-1H-pyrazole-5-carboxamide. A mixture of 3-((8-bromo-6-cyclopropylimidazo[1,2-a]pyridin-2-yl)methyl)-1H-pyrazole-5-carboxylic acid (200 mg, 0.5 mmol, crude), 2,2-dimethoxyethan-1-amine (263 mg, 2.5 mmol), EDCI (384 mg, 2.0 mmol), HOBt (270 mg, 2.0 mmol), and DIPEA (387 mg, 3.0 mmol) in DMF (10 mL) was stirred at 50° C. for 16 h. The reaction was quenched with water (100 mL) and extracted with EtOAc (3×30 mL). The combined organic layers were washed with brine (30 mL), dried over anhydrous Na₂SO₄ and concentrated in vacuo to give the crude, which was purified by column chromatography (eluent: DCM/MeOH=15/1) to give 3-((8-bromo-6-cyclopropylimidazo[1,2-a]pyridin-2-yl)methyl)-N-(2,2-dimethoxy ethyl)-1H-pyrazole-5-carboxamide (140 mg, 63%) as a yellow solid. ESI-MS [M+H]⁺: 448.2.

Synthesis of 2-((8-bromo-6-cyclopropylimidazo[1,2-a]pyridin-2-yl)methyl)pyrazolo[1,5-a]pyrazin-4-ol. A mixture of 3-((8-bromo-6-cyclopropylimidazo[1,2-a]pyridin-2-yl)methyl)-N-(2,2-dimethoxyethyl)-1H-pyrazole-5-carboxamide (140 mg, 0.31 mmol) in TFA (5.0 mL) was stirred at 75° C. for 5 h. The reaction was concentrated in vacuo to give 2-((8-bromo-6-cyclopropylimidazo[1,2-a]pyridin-2-yl)methyl)-pyrazolo[1,5-a]pyrazin-4-ol (110 mg, crude) as a yellow solid which was used for the next step directly. ESI-MS [M+H]+: 384.2.

Synthesis of 2-((8-bromo-6-cyclopropylimidazo[1,2-a]pyridin-2-yl)methyl)-4-chloropyrazolo[1,5-a]pyrazine. A mixture of 2-((8-bromo-6-cyclopropylimidazo[1,2-a]pyridin-2-yl)methyl)pyrazolo-[1,5-a]pyrazin-4-ol (110 mg, 0.29 mmol, crude) in POCl₃ (5.0 mL) was stirred at 90° C. for 3 h. The reaction was concentrated in vacuo to give the crude, which was diluted with EtOAc (50 mL) and quenched with saturated aq·NaHCO₃ (100 mL). The mixture was extracted with EtOAc (3×30 mL). The combined organic layers were washed with brine (30 mL), dried over anhydrous Na₂SO₄, concentrated in vacuo to give the crude, & purified by preparative TLC (eluent: DCM/MeOH=15/1) to give 2-((8-bromo-6-cyclopropylimidazo-[1,2-a]pyridin-2-yl)methyl)-4-chloropyrazolo[1,5-a]pyrazine (85 mg, 74%) as a white solid. ESI-MS [M+H]⁺: 402.1.

Synthesis of (1S,2S)—N-(2-((8-bromo-6-cyclopropylimidazo[1,2-a]pyridin-2-yl)methyl)pyrazolo[1,5-a]pyrazin-4-yl)-2-(4-methylpyrimidin-2-yl)cyclopropane-1-carboxamide. To a mixture of 2-((8-bromo-6-cyclopropylimidazo[1,2-a]pyridin-2-yl)methyl)-4-chloropyrazolo-[1,5-a]pyrazine (85 mg, 0.21 mmol), (1S,2S)-2-(4-methylpyrimidin-2-yl)-cyclopropane-1-carboxamide (41 mg, 0.23 mmol) and Cs₂CO₃ (205 mg, 0.63 mmol) in 1,4-dioxane (10 mL) were added Pd₂(dba)₃ (19 mg, 0.021 mmol) and Xantphos (24 mg, 0.042 mmol). After stirring at 80° C. for 4 h under N₂, the reaction mixture was filtered through Celite® and the filter cake was washed with DCM/MeOH (10/1, 30 mL). The filtrate was concentrated to give the crude, which was purified by preparative TLC (eluent: DCM/MeOH=15/1) to give (1S,2S)—N-(2-((8-bromo-6-cyclopropylimidazo[1,2-a]pyridin-2-yl)methyl)pyrazolo[1,5-a]pyrazin-4-yl)-2-(4-methylpyrimidin-2-yl)cyclopropane-1-carboxamide (55 mg, 48%) as a yellow solid. ESI-MS [M+H]+: 545.2.

Synthesis of (1S,2S)—N-(2-((6-cyclopropyl-8-(3-methyl-2,4-dioxoimidazolidin-1-yl)imidazo[1,2-a]pyridin-2-yl)methyl)pyrazolo[1,5-a]pyrazin-4-yl)-2-(4-methylpyrimidin-2-yl)cyclopropane-1-carboxamide (I-649). To a mixture of (1S,2S)—N-(2-((8-bromo-6-cyclopropylimidazo[1,2-a]pyridin-2-yl)methyl)pyrazolo[1,5-a]pyrazin-4-yl)-2-(4-methylpyrimidin-2-yl)cyclopropane-1-carboxamide (54 mg, 0.1 mmol), 3-methylimidazolidine-2,4-dione (57 mg, 0.5 mmol), and Cs₂CO₃ (98 mg, 0.3 mmol) in 1,4-dioxane (5 mL) were added Pd₂(dba)₃ (18 mg, 0.02 mmol) and Xantphos (23 mg, 0.04 mmol). After stirring at 100° C. for 3 h under N₂, the reaction mixture was filtered through Celite® and the filter cake was washed with DCM/MeOH (10/1, 30 mL). The filtrate was concentrated in vacuo to give the crude, which was purified by preparative HPLC to give (1S,2S)—N-(2-((6-cyclopropyl-8-(3-methyl-2,4-dioxoimidazolidin-1-yl)imidazo[1,2-a]pyridin-2-yl)methyl)pyrazolo[1,5-a]pyrazin-4-yl)-2-(4-methylpyrimidin-2-yl)cyclopropane-1-carboxamide (5 mg, 9%) as a white solid. ESI-MS [M+H]⁺: 577.2. ¹H NMR (400 MHz, DMSO) δ 10.96 (s, 1H), 8.53 (d, J=5.1 Hz, 1H), 8.49 (d, J=4.8 Hz, 1H), 8.23 (s, 1H), 7.69 (s, 1H), 7.57 (d, J=4.5 Hz, 1H), 7.33 (s, 1H), 7.22 (d, J=4.9 Hz, 1H), 6.80 (s, 1H), 4.89 (s, 2H), 4.25 (s, 2H), 2.96 (s, 3H), 2.71-2.67 (m, 2H), 2.42 (s, 3H), 1.99-1.88 (m, 1H), 1.59-1.55 (m, 2H), 0.99-0.87 (m, 2H), 0.66-0.62 (m, 2H).

### Example 650

### Synthesis of (1S,2S)—N-(6-((1-(6-cyclopropyl-8-(3-methyl-2,4-dioxoimidazolidin-1-yl)imidazo[1,2-a]pyridin-2-yl)-2-hydroxyethyl)amino)-2-methylpyrimidin-4-yl)-2-(4-methylpyrimidin-2-yl)cyclopropane-1-carboxamide (I-650)

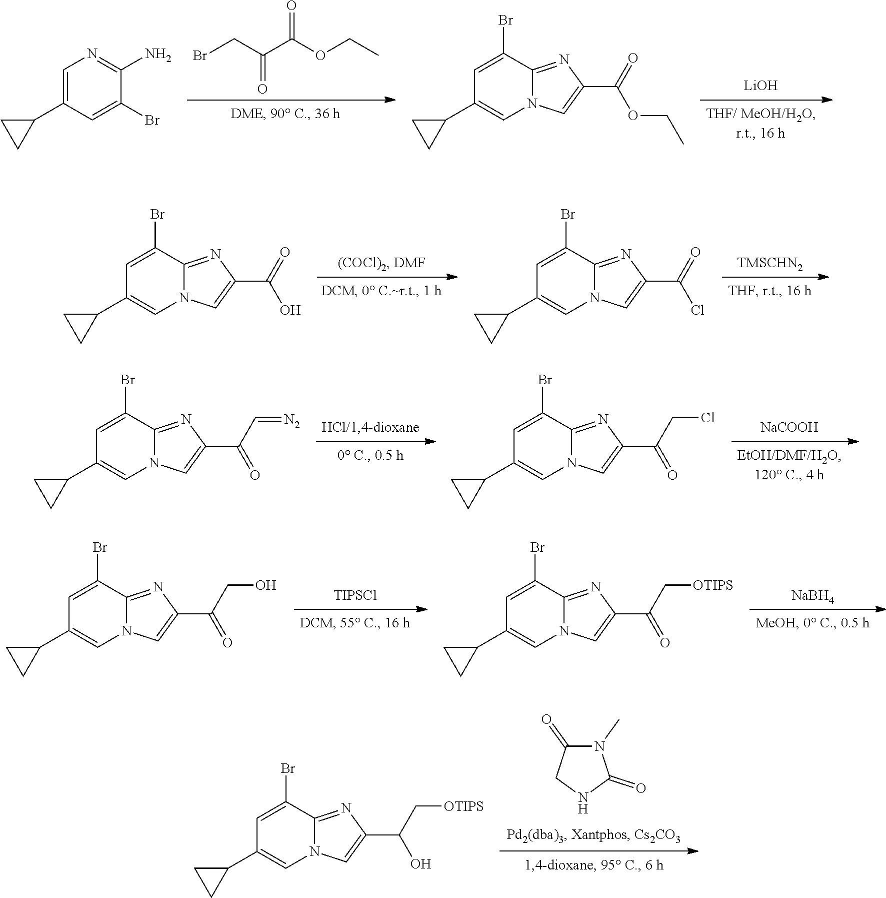

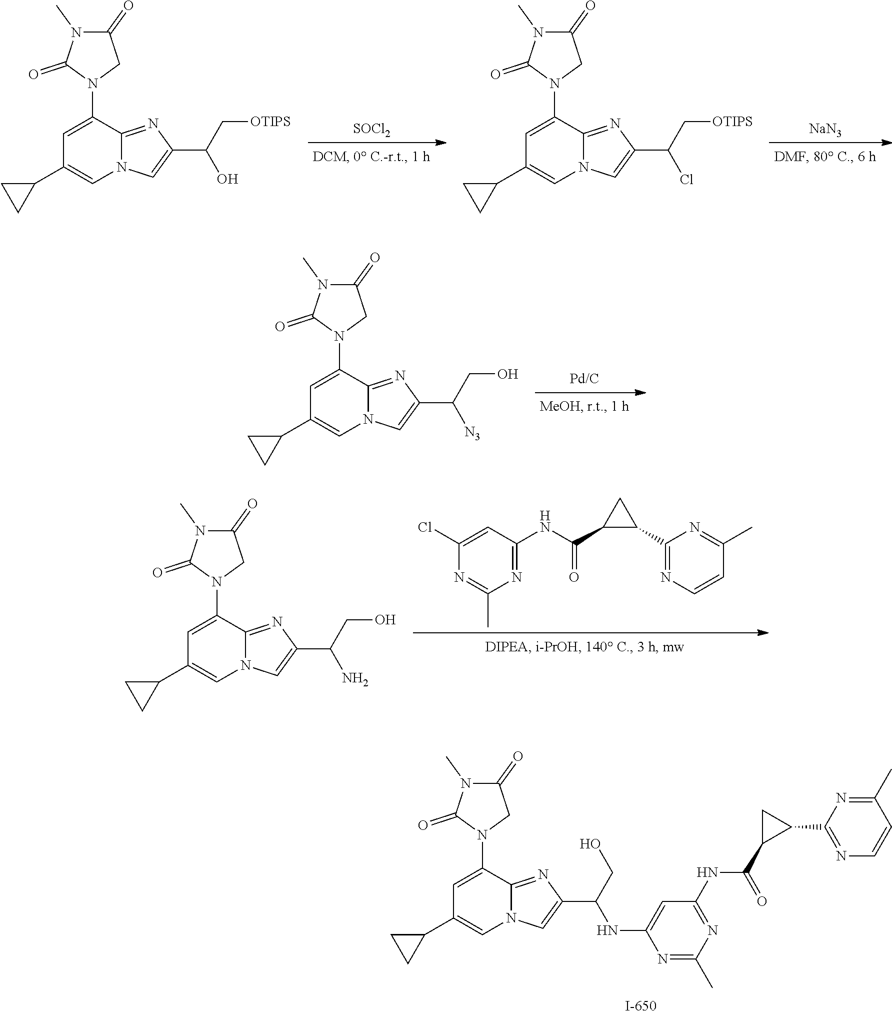

Synthesis of ethyl 8-bromo-6-cyclopropylimidazo[1,2-a]pyridine-2-carboxylate. To a mixture of 3-bromo-5-cyclopropylpyridin-2-amine (6.0 g, 28 mmol) in DME (50 mL) was added ethyl 3-bromo-2-oxopropanoate (6.6 g, 34 mmol). The resulting reaction mixture was stirred at 90° C. for 36 h under N₂. The mixture was quenched with saturated aq·NaHCO₃ (100 mL) and extracted with EtOAc (3×50 mL). The combined organic layers were washed with brine (50 mL), dried over Na₂SO₄ and concentrated in vacuo. The residue was purified by flash column chromatography (eluent: EtOAc/PE=0-50%) to afford ethyl 8-bromo-6-cyclopropylimidazo[1,2-a]pyridine-2-carboxylate (5.8 g, 67%) as yellow solid. ESI-MS [M+H]+: 309.0.

Synthesis of 8-bromo-6-cyclopropylimidazo[1,2-a]pyridine-2-carboxylic acid. A mixture of ethyl 8-bromo-6-cyclopropylimidazo[1,2-a]pyridine-2-carboxylate (4 g, 12.9 mmol) and LiOH—H₂O (1.09 g, 25.8 mmol) in THF/MeOH/H₂O (40 mL/40 mL/16 mL) was stirred at room temperature for 16 h under N₂. The reaction mixture was diluted with water (50 mL) and extracted with EtOAc (3×50 mL). The aqueous phase was adjusted to pH=4 with HCl (1N) and filtered. The solid was dried to afford 8-bromo-6-cyclopropylimidazo[1,2-a]pyridine-2-carboxylic acid (3.0 g, 83%) as yellow solid. ESI-MS [M+H]+: 281.0. pH˜3

Synthesis of 8-bromo-6-cyclopropylimidazo[1,2-a]pyridine-2-carbonyl chloride. To a mixture of 8-bromo-6-cyclopropylimidazo[1,2-a]pyridine-2-carboxylic acid (2.0 g, 7.1 mmol) in DCM (50 mL) was added oxalyl chloride (1.1 g, 8.5 mmol) and DMF (2 drops) at 0° C. The mixture was stirred at room temperature for 1 h and then concentrated to afford 8-bromo-6-cyclopropylimidazo[1,2-a]pyridine-2-carbonyl chloride (2.1 g, quant) as yellow oil which was used in next step directly. ESI-MS [M+H]+: 295.0.

Synthesis of 1-(8-bromo-6-cyclopropylimidazo[1,2-a]pyridin-2-yl)-2-diazoethan-1-one. To a mixture of 8-bromo-6-cyclopropylimidazo[1,2-a]pyridine-2-carbonyl chloride (2.1 g, 7.1 mmol) in THF (50 mL) was added TMSCHN₂ (7.1 mL, 14 mmol) at 0° C. The mixture was stirred at room temperature for 16 h and then concentrated in vacuo to afford 1-(8-bromo-6-cyclopropylimidazo[1,2-a]pyridin-2-yl)-2-diazoethan-1-one (2.2 g, quant) as yellow oil which was used in next step directly. ESI-MS [M+H]+: 305.0.

Synthesis of 1-(8-bromo-6-cyclopropylimidazo[1,2-a]pyridin-2-yl)-2-chloroethan-1-one. To a mixture of 1-(8-bromo-6-cyclopropylimidazo[1,2-a]pyridin-2-yl)-2-diazoethan-1-one (2.1 g, 7.1 mmol) in THF (50 mL) was added HCl (2.1 mL, 4.0 M) at 0° C. The mixture was stirred at 0° C. for 0.5 h. The reaction mixture was quenched with water (100 mL) and extracted with EtOAc (3×50 mL). The combined organic layers were washed with brine (50 mL), dried over Na₂SO₄ and concentrated. The residue was purified by column chromatography (eluent: EtOAc/PE, 0-50%) to afford 1-(8-bromo-6-cyclopropylimidazo[1,2-a]pyridin-2-yl)-2-chloroethan-1-one (1.0 g, 47%, for three steps) as yellow solid. ESI-MS [M+H]+: 313.0.

Synthesis of 1-(8-bromo-6-cyclopropylimidazo[1,2-a]pyridin-2-yl)-2-hydroxyethan-1-one. A mixture of 1-(8-bromo-6-cyclopropylimidazo[1,2-a]pyridin-2-yl)-2-chloroethan-1-one (1.1 g, 3.5 mmol) and HCOONa (0.72 g, 11 mmol) in EtOH/DMF/water (28 mL/10 mL/8 mL) was stirred at 120° C. for 4 h under N₂. After cooling to room temperature, water (40 mL) was added and the mixture was extracted by EtOAc (3×40 mL). The combined organic layers were washed with brine (50 mL), dried over Na₂SO₄ and concentrated in vacuo. The residue was purified by column chromatography (eluent: EtOAc/PE=0-50%) to afford 1-(8-bromo-6-cyclopropylimidazo[1,2-a]pyridin-2-yl)-2-hydroxyethan-1-one (0.54 g, 52%) as pale solid. ESI-MS [M+H]+: 295.1.

Synthesis of 1-(8-bromo-6-cyclopropylimidazo[1,2-a]pyridin-2-yl)-2-((triisopropyl silyl)oxy)ethan-1-one. To a mixture of 1-(8-bromo-6-cyclopropylimidazo[1,2-a] pyridin-2-yl)-2-hydroxyethan-1-one (0.54 g, 1.8 mmol) and imidazole (0.87 g, 13 mmol) in DCM (30 mL) was added TIPSCl (1.8 g, 9.2 mmol). After stirring at 55° C. for 16 h under N₂, the reaction mixture was quenched with H2O (100 mL) and extracted with DCM (3×30 mL). The combined organic layers were washed with brine (100 mL), dried over Na₂SO₄ and concentrated in vacuo. The residue was purified by column chromatography (eluent: EtOAc/PE from 0 to 50%) to afford 1-(8-bromo-6-cyclopropylimidazo[1,2-a]pyridin-2-yl)-2-((triisopropylsilyl)oxy)ethan-1-one (0.69 g, 84%) as pale solid. ESI-MS [M+H]+: 451.1.

Synthesis of 1-(8-bromo-6-cyclopropylimidazo[1,2-a]pyridin-2-yl)-2-((triisopropylsilyl)oxy) ethan-1-ol. To a mixture of 1-(8-bromo-6-cyclopropylimidazo[1,2-a]pyridin-2-yl)-2-((triisopropyl silyl) oxy)ethan-1-one (0.69 g, 1.5 mmol) in MeOH (20 mL) was added NaBH₄ (0.29 mg, 7.7 mmol) at 0° C. After stirring at 0° C. for 0.5 h under N₂, the reaction mixture was quenched by H2O (30 mL) and extracted with DCM (3×30 mL). The combined organic layers were washed with brine (30 mL), dried over Na₂SO₄ and concentrated in vacuo. The residue was purified by column chromatography (eluent: EtOAc/PE from 0-50%) to afford 1-(8-bromo-6-cyclopropyl imidazo[1,2-a]pyridin-2-yl)-2-((triisopropylsilyl)oxy)ethan-1-ol (0.65 g, 94%) as pale solid. ESI-MS [M+H]+: 453.1.

Synthesis of 1-(6-cyclopropyl-2-(1-hydroxy-2-((triisopropylsilyl)oxy)ethyl)imidazo[1,2-a]pyridin-8-yl)-3-methylimidazolidine-2,4-dione. To a mixture of 1-(8-bromo-6-cyclopropyl imidazo[1,2-a]pyridin-2-yl)-2-((triisopropylsilyl)oxy)ethan-1-ol (0.65 g, 1.4 mmol), 3-methyl imidazolidine-2,4-dione (0.49 g, 4.3 mmol), and Cs₂CO₃ (1.4 g, 4.3 mmol) in 1,4-dioxane (40 mL) were added Pd₂(dba)₃ (0.26 g, 0.29 mmol) and Xantphos (0.55 mg, 0.57 mmol). After stirring at 95° C. for 6 h under N₂, the reaction mixture was filtered through Celite® and the filter cake was washed with DCM/MeOH (10/1, 100 mL). The filtrate was concentrated in vacuo to give the crude which was purified by column chromatography (eluent: EtOAc/PE from 0 to 50%) to afford 1-(6-cyclo propyl-2-(1-hydroxy-2-((triisopropylsilyl)oxy)ethyl)imidazo[1,2-a]pyridin-8-yl)-3-methyl imidazolidine-2,4-dione (0.82 g, quant) as yellow oil. ESI-MS [M+H]+: 487.3.

Synthesis of 1-(2-(1-chloro-2-((triisopropylsilyl)oxy)ethyl)-6-cyclopropylimidazo[1,2-a]pyridin-8-yl)-3-methylimidazolidine-2,4-dione. To a mixture of 1-(6-cyclopropyl-2-(1-hydroxy-2-((triisopropylsilyl)oxy)ethyl)imidazo[1,2-a]pyridin-8-yl)-3-methylimidazolidine-2,4-dione (0.82 g, 1.7 mmol) in DCM (20 mL) was added SOCl₂ (0.40 g, 3.4 mmol) at 0° C. The mixture was stirred at room temperature for 1 h under N₂ and then concentrated in vacuo to afford 1-(2-(1-chloro-2-((triiso propylsilyl)oxy)ethyl)-6-cyclopropylimidazo[1,2-a]pyridin-8-yl)-3-methylimidazolidine-2,4-dione (0.85 g, quant) as yellow oil which was used in next step directly. ESI-MS [M+H]+: 505.2.

Synthesis of 1-(2-(1-azido-2-hydroxyethyl)-6-cyclopropylimidazo[1,2-a]pyridin-8-yl)-3-methylimidazolidine-2,4-dione. To a mixture of 1-(2-(1-chloro-2-((triisopropylsilyl)oxy)ethyl)-6-cyclopropylimidazo[1,2-a]pyridin-8-yl)-3-methylimidazolidine-2,4-dione (0.85 g, 1.7 mmol) in DMF (20 mL) was added NaN₃ (0.55 g, 8.4 mmol). After stirring at 80° C. for 6 h under N₂, the reaction mixture was quenched with water (200 mL) and extracted with EtOAc (3×60 mL). The combined organic layers were washed with brine (60 mL), dried over Na₂SO₄ and concentrated in vacuo. The residue was purified by column chromatography (eluent: EtOAc/PE from 0 to 50%) to afford 1-(2-(1-azido-2-hydroxyethyl)-6-cyclopropylimidazo[1,2-a]pyridin-8-yl)-3-methyl imidazolidine-2,4-dione (0.18 g, 24% for two steps) as yellow solid. ESI-MS [M+H]+: 356.2.

Synthesis of 1-(2-(1-amino-2-hydroxyethyl)-6-cyclopropylimidazo[1,2-a]pyridin-8-yl)-3-methylimidazolidine-2,4-dione. A mixture of 1-(2-(1-azido-2-hydroxyethyl)-6-cyclopropylimidazo[1,2-a]pyridin-8-yl)-3-methylimidazolidine-2,4-dione (0.21 g, 0.59 mmol) and Pd/C (0.21 g) in MeOH (10 mL) was stirred at RT for 1 h. The reaction mixture was filtered through Celite® and the filter cake was washed with MeOH (10 mL). The filtrate was concentrated in vacuo to afford 1-(2-(1-amino-2-hydroxyethyl)-6-cyclopropylimidazo[1,2-a]pyridin-8-yl)-3-methylimidazolidine-2,4-dione (170 mg, crude) as yellow oil which was used in next step directly. ESI-MS [M+H]+: 330.1.

Synthesis of (1S,2S)—N-(6-((1-(6-cyclopropyl-8-(3-methyl-2,4-dioxoimidazolidin-1-yl) imidazo[1,2-a]pyridin-2-yl)-2-hydroxyethyl)amino)-2-methylpyrimidin-4-yl)-2-(4-methyl pyrimidin-2-yl)cyclopropane-1-carboxamide (I-650). A mixture of 1-(2-(1-amino-2-hydroxyethyl)-6-cyclo propylimidazo[1,2-a]pyridin-8-yl)-3-methylimidazolidine-2,4-dione (88 mg, 0.27 mmol), (1S,2S)—N-(6-chloro-2-methylpyrimidin-4-yl)-2-(4-methylpyrimidin-2-yl)cyclopropane-1-carbox amide (81 mg, 0.27 mmol), and DIPEA (0.10 g, 0.81 mmol) in IPA (3 mL) in a sealed tube was degassed with N₂ for 1 min. After heating in a microwave reactor at 140° C. for 3 h, the mixture quenched with H2O (50 mL) and extracted with EtOAc (3×50 mL). The combined organic layers were washed with brine (50 mL), dried over Na₂SO₄ and concentrated in vacuo. The residue was purified by preparative TLC (eluent: DCM:MeOH=20:1) to afford (1S,2S)—N-(6-((1-(6-cyclo propyl-8-(3-methyl-2,4-dioxoimi-dazolidin-1-yl)imidazo[1,2-a]pyridin-2-yl)-2-hydroxyethyl) amino) -2-methylpyrimidin-4-yl)-2-(4-methylpyrimidin-2-yl)cyclopropane-1-carboxamide (21 mg, 13%) as pale solid. ESI-MS [M+H]+: 597.3. ¹HNMR (400 MHz, DMSO) δ 10.64 (s, 1H), 8.52 (d, J=5.1 Hz, 1H), 8.24 (s, 1H), 7.69 (d, J=2.7 Hz, 1H), 7.61 (s, 1H), 7.34 (s, 1H), 7.21-7.16 (m, 2H), 5.39 (s, 1H), 4.96-4.93 (m, 2H), 4.82 (s, 1H), 3.86-3.81 (m, 1H), 3.74-3.68 (m, 1H), 2.98 (d, J=1.2 Hz, 3H), 2.57-2.56 (m, 2H), 2.41 (s, 3H), 2.24 (s, 3H), 1.95-1.92 (m, 1H), 1.53-1.46 (m, 2H), 0.94-0.91 (m, 2H), 0.66-0.62 (m, H).

### Example 651

### Synthesis of (1S,2S)—N-(5-(((6-cyclopropyl-8-(3-methyl-2,4-dioxoimidazolidin-1-yl)imidazo[1,2-a]pyridin-2-yl)methyl)amino)-1,2,4-triazin-3-yl)-2-(4-methylpyrimidin-2-yl)cyclopropane-1-carboxamide (I-651)

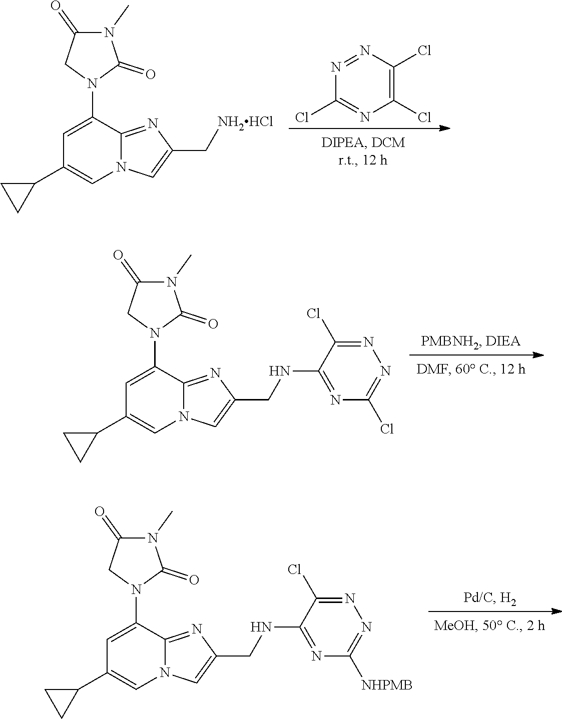

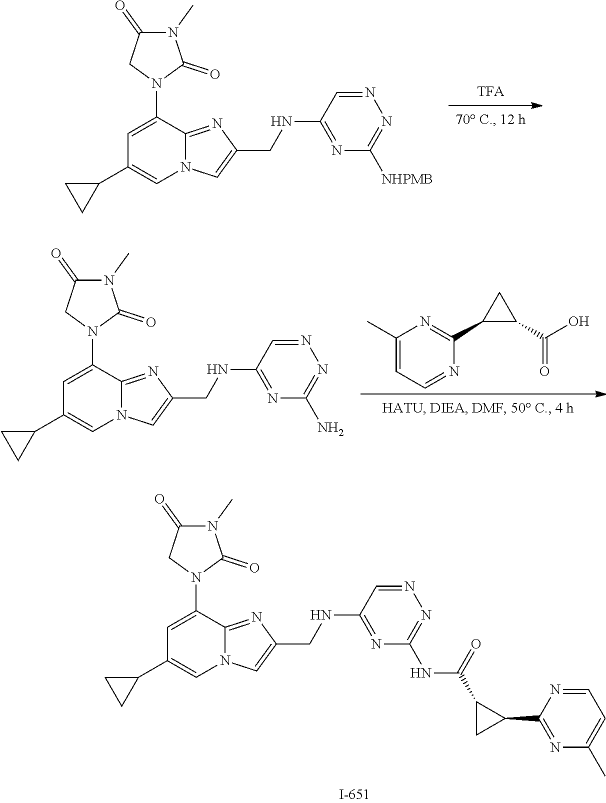

Synthesis of 1-(6-cyclopropyl-2-(((3,6-dichloro-1,2,4-triazin-5-yl)amino)methyl)imidazo[1,2-a]pyridin-8-yl)-3-methylimidazolidine-2,4-dione. A mixture of 1-(2-(aminomethyl)-6-cyclopropylimidazo[1,2-a]pyridin-8-yl)-3-methylimidazolidine-2,4-dione hydrochloride (300 mg, 0.89 mmol), 3,5,6-trichloro-1,2,4-triazine (197 mg, 1.07 mmol), and DIPEA (344 mg, 2.67 mmol) in DCM (6 mL) was stirred at room temperature for 12 h. The mixture quenched with saturated aq·NaHCO₃ (100 mL) and extracted with EtOAc (3×50 mL). The combined organic layers were washed with brine (50 mL), dried over Na₂SO₄ and concentrated in vacuo. The residue was purified by preparative TLC (eluent: DCM/MeOH=20/1) to give 1-(6-cyclopropyl-2-(((3,6-dichloro-1,2,4-triazin-5-yl)amino)methyl)imidazo[1,2-a]pyridin-8-yl)-3-methylimidazolidine-2,4-dione (300 mg, 70%) as a yellow solid. ESI-MS [M+H]+: 447.2.

Synthesis of 1-(2-(((6-chloro-3-((4-methoxybenzyl)amino)-1,2,4-triazin-5-yl)amino)methyl)-6-cyclopropylimidazo[1,2-a]pyridin-8-yl)-3-methylimidazolidine-2,4-dione. To a mixture of 1-(6-cyclopropyl-2-(((3,6-dichloro-1,2,4-triazin-5-yl)amino)methyl)imidazo[1,2-a]pyridin-8-yl)-3-methylimidazolidine-2,4-dione (300 mg, 0.67 mmol), DIPEA (258 mg, 2.0 mmol) in DMF (5 mL) was added PMBNH₂ (275.7 mg, 2.0 mmol). The reaction mixture was stirred at 60° C. for 12 h under N₂. Water (50 mL) was added and the mixture was extracted with EtOAc (30 mL×3). The combined organic layers were washed with brine (30 mL), dried over Na₂SO₄ and concentrated in vacuo to give the crude, which was purified by preparative TLC (eluent: DCM/MeOH=20/1) to give 1-(2-(((6-chloro-3-((4-methoxybenzyl)amino)-1,2,4-triazin-5-yl)amino)methyl)-6-cyclopropylimidazo[1,2-a]pyridin-8-yl)-3-methylimidazolidine-2,4-dione (300 mg, 82%) as a yellow solid. ESI-MS [M+H]+: 584.2

Synthesis of 1-(6-cyclopropyl-2-(((3-((4-methoxybenzyl)amino)-1,2,4-triazin-5-yl)amino)methyl)imidazo[1,2-a]pyridin-8-yl)-3-methylimidazolidine-2,4-dione. A mixture of 1-(2-(((6-chloro-3-((4-methoxybenzyl)amino)-1,2,4-triazin-5-yl)amino)methyl)-6-cyclopropylimidazo[1,2-a]pyridin-8-yl)-3-methylimidazolidine-2,4-dione (200 mg, 0.36 mmol) and Pd/C (100 mg) in MeOH (5 mL) was stirred at 50° C. for 2 h under H₂. The reaction mixture was filtered through Celite® and the filter cake was washed with DCM/MeOH (10/1, 50 mL). The filtrate was concentrated to give the crude, which was purified by preparative TLC (eluent: DCM/MeOH=20/1) to give 1-(6-cyclopropyl-2-(((3-((4-methoxybenzyl)amino)-1,2,4-triazin-5-yl)amino)methyl)imidazo[1,2-a]pyridin-8-yl)-3-methylimidazolidine-2,4-dione (150 mg, 80%) as a yellow solid. ESI-MS [M+H]+: 514.2

Synthesis of 1-(6-cyclopropyl-2-(((3-((4-methoxybenzyl)amino)-1,2,4-triazin-5-yl)amino)methyl)imidazo[1,2-a]pyridin-8-yl)-3-methylimidazolidine-2,4-dione. A mixture of 1-(2-(((6-(bis(3,4-dimethylbenzyl)amino)-3-(trifluoromethyl)pyridazin-4-yl)oxy)methyl)-6-cyclopropylimidazo[1,2-a]pyridin-8-yl)-3-methylimidazolidine-2,4-dione (300 mg, 0.58 mmol) in TFA (4 ml) was stirred at 70° C. for 12 h. The mixture was quenched with saturated aq·NaHCO₃ (50 mL) and extracted with EtOAc (3×30 mL). The combined organic layers were washed with brine (30 mL), dried over anhydrous Na₂SO₄, and concentrated in vacuo to give the crude product, which was purified by preparative TLC (eluent: DCM/MeOH=10/1) to give 1-(2-(((3-amino-1,2,4-triazin-5-yl)amino)methyl)-6-cyclopropylimidazo[1,2-a]pyridin-8-yl)-3-methylimidazolidine-2,4-dione (140 mg, 61%) as a white solid. ESI-MS [M+H]+: 394.2.

Synthesis of (1S,2S)—N-(5-(((6-cyclopropyl-8-(3-methyl-2,4-dioxoimidazolidin-1-yl)imidazo[1,2-a]pyridin-2-yl)methyl)amino)-1,2,4-triazin-3-yl)-2-(4-methylpyrimidin-2-yl)cyclopropane-1-carboxamide (I-561). A mixture of 1-(2-(((3-amino-1,2,4-triazin-5-yl)amino)methyl)-6-cyclopropylimidazo[1,2-a]pyridin-8-yl)-3-methylimidazolidine-2,4-dione (140 mg, 0.35 mmol), (1S,2S)-2-(4-methylpyrimidin-2-yl)cyclopropane-1-carboxylic acid (70 mg, 0.39 mmol), HATU (200 mg, 0.52 mmol), and DIPEA (135 mg, 1.05 mmol) in DMF (4 mL) was stirred at 50° C. for 4 h. Water (50 mL) was added and extracted with EtOAc (3×30 mL). The combined organic layers were washed with brine (30 mL), dried over Na₂SO₄, filtered and concentrated in vacuo to give the crude, which was purified by preparative TLC (eluent: DCM/MeOH=20/1) to give (1S,2S)—N-(5-(((6-cyclopropyl-8-(3-methyl-2,4-dioxoimidazolidin-1-yl)imidazo[1,2-a]pyridin-2-yl)methyl)amino)-1,2,4-triazin-3-yl)-2-(4-methylpyrimidin-2-yl)cyclopropane-1-carboxamide (95 mg, 50%) as a yellow solid. ESI-MS [M+H]+: 554.2 ¹H NMR (400 MHz, DMSO) δ 10.63 (s, 1H), 8.65 (s, 1H), 8.48 (d, J=5.1 Hz, 1H), 8.33 (s, 1H), 8.23 (d, J=8.6 Hz, 1H), 7.83 (s, 1H), 7.34 (s, 1H), 7.15 (d, J=5.1 Hz, 1H), 4.89 (s, 2H), 4.54 (d, J=5.2 Hz, 2H), 2.97 (s, 3H), 2.69-2.59 (m, 2H), 2.38 (s, 3H), 1.98-1.90 (m, 1H), 1.59-1.47 (m, 2H), 0.98-0.90 (m, 2H), 0.70-0.60 (m, 2H).

### Example 652

### Synthesis of (1S,2S)—N-(2-((6-cyclopropyl-8-(3-methyl-2,4-dioxoimidazolidin-1-yl)imidazo[1,2-a]pyridin-2-yl)methoxy)-6-methylpyrimidin-4-yl)-2-(4-methylpyrimidin-2-yl)cyclopropane-1-carboxamide (I-652)

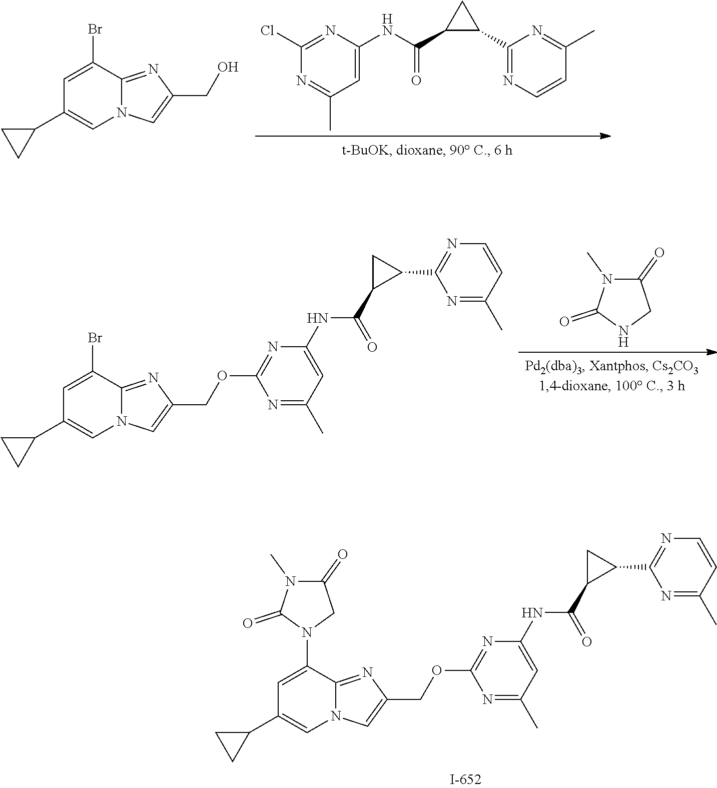

Synthesis of (1S,2S)—N-(2-((8-bromo-6-cyclopropylimidazo[1,2-a]pyridin-2-yl)methoxy)-6-methylpyrimidin-4-yl)-2-(4-methylpyrimidin-2-yl)cyclopropane-1-carboxamide. To a mixture of (8-bromo-6-cyclopropylimidazo[1,2-a]pyridin-2-yl)methanol (0.12 g, 0.44 mmol) and (1S,2S)—N-(2-chloro-6-methylpyrimidin-4-yl)-2-(4-methylpyrimidin-2-yl)cyclopropane-1-carboxamide (0.15 g, 0.51 mmol) in 1,4-dioxane (4.0 mL) was added t-BuOK (0.88 mL, 0.88 mmol, 1M in THF). The mixture was stirred at 90° C. for 6 h and cooled to room temperature. The reaction was quenched with saturated aq·NH₄Cl (50 mL) and extracted with EtOAc (3×50 mL). The combined organic layers were washed with brine (50 mL), dried over Na₂SO₄ and concentrated in vacuo. The residue was purified by flash column chromatography (eluent: MeOH/DCM from 0 to 5%) to afford (1S,2S)—N-(2-((8-bromo-6-cyclopropylimidazo[1,2-a]pyridin-2-yl)methoxy)-6-methylpyrimidin-4-yl)-2-(4-methylpyrimidin-2-yl)cyclopropane-1-carboxamide (0.12 mg, yield 49%) as white solid. ESI-MS [M+H]+: 534.1

Synthesis of (1S,2S)—N-(2-((6-cyclopropyl-8-(3-methyl-2,4-dioxoimidazolidin-1-yl)imidazo[1,2-a]pyridin-2-yl)methoxy)-6-methylpyrimidin-4-yl)-2-(4-methylpyrimidin-2-yl)cyclopropane-1-carboxamide (I-652). A mixture of (1S,2S)—N-(2-((8-bromo-6-cyclopropylimidazo[1,2-a]pyridin-2-yl)methoxy)-6-methylpyrimidin-4-yl)-2-(4-methylpyrimidin-2-yl)cyclopropane-1-carboxamide (60 mg, 0.11 mmol), 3-methylimidazolidine-2,4-dione (64 mg, 0.56 mmol), Pd₂(dba)₃ (31 mg, 0.034 mmol), Xantphos (39 mg, 0.067 mmol), and Cs₂CO₃ (0.11 g, 0.34 mmol) in 1,4-dioxane (4.0 mL) was stirred at 100° C. for 3 h under N₂. The mixture was filtered through Celite® and the filter cake was washed with DCM/MeOH (10/1, 100 mL). The filtrate was concentrated in vacuo to give the crude, which was purified by preparative TLC (eluent: 100% EtOAc) to afford (1S,2S)—N-(2-((6-cyclopropyl-8-(3-methyl-2,4-dioxoimidazolidin-1-yl)imidazo[1,2-a]pyridin-2-yl)methoxy)-6-methylpyrimidin-4-yl)-2-(4-methylpyrimidin-2-yl)cyclopropane-1-carboxamide (21.2 mg, yield 33%) as white solid. ESI-MS [M+H]+: 568.2. ¹H NMR (400 MHz, DMSO) δ 11.11 (s, 1H), 8.53 (d, J=5.1 Hz, 1H), 8.28 (d, J=1.3 Hz, 1H), 7.92 (s, 1H), 7.63 (s, 1H), 7.37 (d, J=1.4 Hz, 1H), 7.21 (d, J=5.1 Hz, 1H), 5.42 (s, 2H), 4.88 (s, 2H), 2.96 (s, 3H), 2.59-2.54 (m, 2H), 2.42 (s, 3H), 2.37 (s, 3H), 1.97-1.93 (m, 1H), 1.59-1.53 (m, 2H), 0.96-0.92 (m, 2H), 0.67-0.64 (m, 2H).

### Example 653

### Synthesis of (1S,2S)—N-(4-(2-(6-cyclopropyl-8-(3-methyl-2,4-dioxoimidazolidin-1-yl)imidazo[1,2-a]pyridin-2-yl)ethyl)pyridin-2-yl)-2-(4-methylpyrimidin-2 l)cyclopropane-1-carboxamide (I-653)

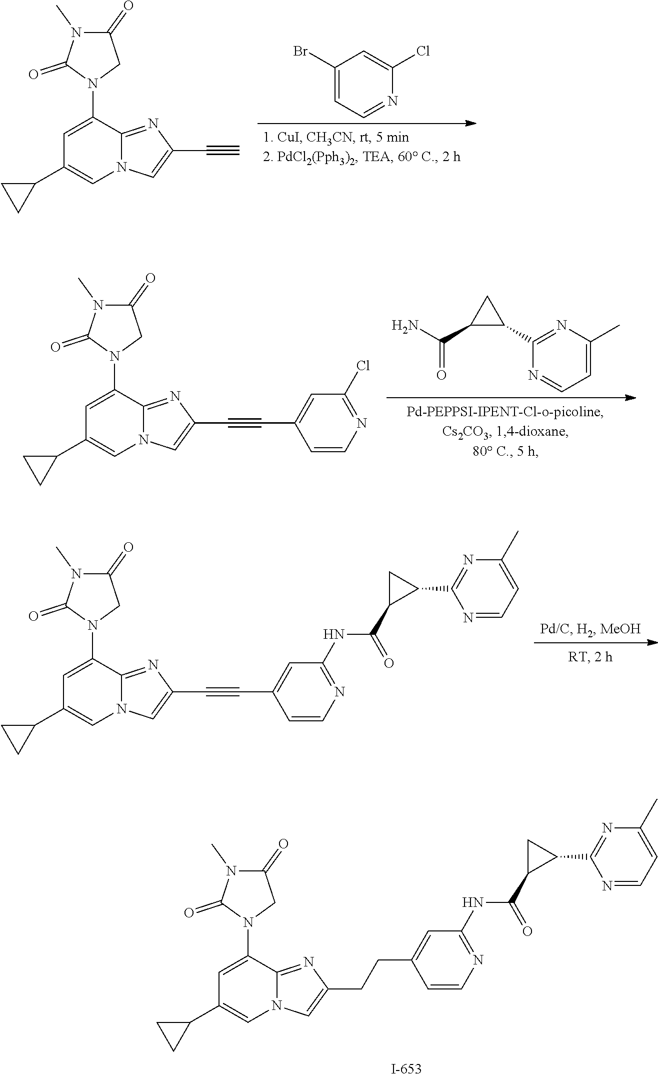

Synthesis of 1-(2-((2-chloropyridin-4-yl)ethynyl)-6-cyclopropylimidazo[1,2-a]pyridin-8-yl)-3-methylimidazolidine-2,4-dione. To a mixture of 1-(6-cyclopropyl-2-ethynylimidazo[1,2-a]pyridin-8-yl)-3-methylimidazolidine-2,4-dione (100 mg, 0.3 mmol) in MeCN (20 mL) was added 4-bromo-2-chloropyridine (98 mg, 0.45 mmol) and CuI (10 mg, 0.9 mmol) at room temperature for 5 min, followed by PdCl₂(PPh3)2 (40 mg, 0.06 mmol), TEA (1 mL). After stirring at 60° C. for 2 h, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to 1-(2-((2-chloropyridin-4-yl)ethynyl)-6-cyclopropylimidazo[1,2-a]pyridin-8-yl)-3-methylimidazolidine-2,4-dione (80 mg, 65%) as a yellow solid. ESI-MS [M+H]+: 406.2.

Synthesis of (1S,2S)—N-(4-((6-cyclopropyl-8-(3-methyl-2,4-dioxoimidazolidin-1-yl)imidazo[1,2-a]pyridin-2-yl)ethynyl)pyridin-2-yl)-2-(4-methylpyrimidin-2-yl)cyclopropane-1-carboxamide. To a mixture of 1-(2-((2-chloropyridin-4-yl)ethynyl)-6-cyclopropylimidazo[1,2-a]pyridin-8-yl)-3-methylimidazolidine-2,4-dione (80 mg, 0.19 mmol), (1S,2S)-2-(4-methylpyrimidin-2-yl)cyclopropane-1-carboxamide (34 mg, 0.19 mmol), and Cs₂CO₃ (185 mg, 0.57 mmol) in 1,4-dioxane (10 mL) was added Pd-PEPPSI-IPENT-Cl-o-picoline (16 mg, 0.02 mmol). The reaction mixture was stirred at 80° C. for 5 h under N₂. The reaction mixture was filtered through Celite® and the filter cake was washed with DCM/MeOH (10/1, 50 mL). The filtrate was concentrated in vacuo to give the crude, which was purified by preparative HPLC to give (1S,2S)—N-(4-((6-cyclopropyl-8-(3-methyl-2,4-dioxoimidazolidin-1-yl)imidazo[1,2-a]pyridin-2-yl)ethynyl)pyridin-2-yl)-2-(4-methylpyrimidin-2-yl)cyclopropane-1-carboxamide (22 mg, 21%) as a white solid. ESI-MS [M+H]⁺: 547.1.

Synthesis of (1S,2S)—N-(4-(2-(6-cyclopropyl-8-(3-methyl-2,4-dioxoimidazolidin-1-yl)imidazo[1,2-a]pyridin-2-yl)ethyl)pyridin-2-yl)-2-(4-methylpyrimidin-2-yl)cyclopropane-1-carboxamide (I-653). A mixture of (1S,2S)—N-(4-((6-cyclopropyl-8-(3-methyl-2,4-dioxoimidazolidin-1-yl)imidazo[1,2-a]pyridin-2-yl)ethynyl)pyridin-2-yl)-2-(4-methylpyrimidin-2-yl)cyclopropane-1-carboxamide (22 mg, 0.04 mmol) and Pd/C (20 mg) in MeOH (5 mL) was stirred at room temperature for 2 h under H₂. The reaction mixture was filtered through Celite® and the filter cake was washed with DCM/MeOH (10/1, 20 mL). The filtrate was concentrated in vacuo to give the crude, which was purified by preparative HPLC to give (1S,2S)—N-(4-(2-(6-cyclopropyl-8-(3-methyl-2,4-dioxoimidazolidin-1-yl)imidazo[1,2-a]pyridin-2-yl)ethyl)pyridin-2-yl)-2-(4-methylpyrimidin-2-yl)cyclopropane-1-carboxamide (18 mg, 81%) as a white solid. ESI-MS: [M+H]+, 551.2. ¹H NMR (400 MHz, DMSO) δ 10.75 (s, 1H), 8.53 (d, J=5.1 Hz, 1H), 8.20 (d, J=1.1 Hz, 1H), 8.15 (d, J=5.0 Hz, 1H), 8.01 (s, 1H), 7.62 (s, 1H), 7.31 (d, J=1.5 Hz, 1H), 7.21 (d, J=5.1 Hz, 1H), 6.99 (dd, J=5.1, 1.4 Hz, 1H), 4.92 (s, 2H), 3.00 (s, 4H), 2.98 (s, 3H), 2.68-2.61 (m, 1H), 2.56-2.53 (m, 1H), 2.42 (s, 3H), 1.97-1.92 (m, 1H), 1.53-1.47 (m, 2H), 0.96-0.91 (m, 2H), 0.67-0.63 (m, 2H).

### Example 654

### Synthesis of ethyl 4-((6-cyclopropyl-8-(3-methyl-2,4-dioxoimidazolidin-1-yl)imidazo[1,2-a]pyridin-2-yl)methoxy)-2-((1S,2S)-2-(4-methylpyrimidin-2-yl)cyclopropane-1-carboxamido)pyrimidine-5-carboxylate (I-654)

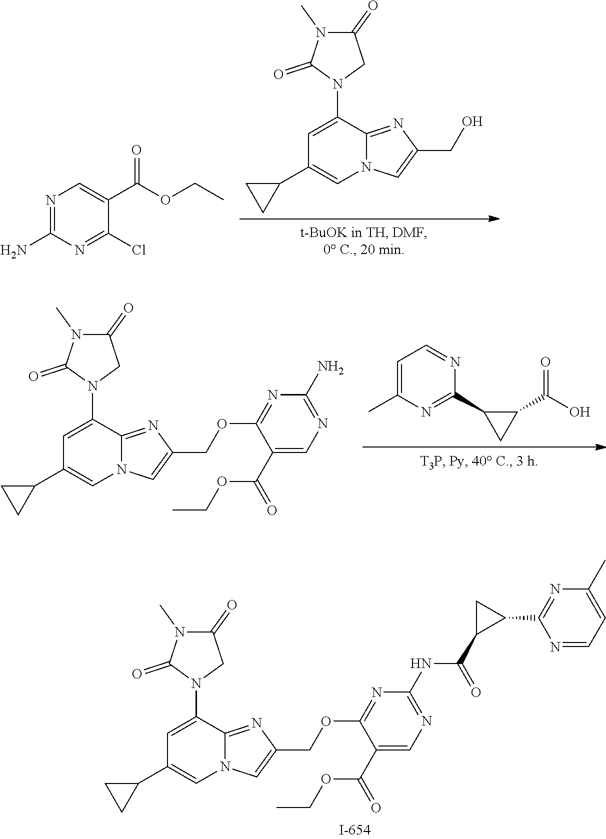

Synthesis of ethyl 2-amino-4-((6-cyclopropyl-8-(3-methyl-2,4-dioxoimidazolidin-1-yl) imidazo[1,2-a]pyridin-2-yl)methoxy)pyrimidine-5-carboxylate. To a solution of 1-(6-cyclopropyl-2-(hydroxymethyl)imidazo[1,2-a]pyridin-8-yl)-3-methylimidazolidine-2,4-dione (140 mg, 0.47 mmol) and ethyl 2-amino-4-chloropyrimidine-5-carboxylate (95 mg, 0.47 mmol) in DMF (10 mL) was added t-BuOK (1M in THF, 0.94 mL) at 0° C. After stirring at 0° C. for 20 min, the mixture was quenched with water (10 mL) and extracted with EtOAc (3×20 mL). The combined organic layers were washed with brine (30 mL), dried over Na₂SO₄, and concentrated in vacuo to give the crude product, which was purified with Pre-TLC (eluent: DCM/MeOH=15/1) to give the product ethyl 2-amino-4-((6-cyclopropyl-8-(3-methyl-2,4-dioxoimidazolidin-1-yl)imidazo[1,2-a]pyridin-2-yl)methoxy)pyrimidine-5-carboxylate (40 mg, 18%) as a white solid. ESI-MS [M+H]+: 466.1

Synthesis of ethyl 4-((6-cyclopropyl-8-(3-methyl-2,4-dioxoimidazolidin-1-yl)imidazo[1,2-a]pyridin-2-yl)methoxy)-2-((1S,2S)-2-(4-methylpyrimidin-2-yl)cyclopropane-1-carboxamido)pyrimidine-5-carboxylate (I-654). To a mixture of ethyl 2-amino-4-((6-cyclopropyl-8-(3-methyl-2,4-dioxoimidazolidin-1-yl)imidazo[1,2-a]pyridin-2-yl)methoxy)pyrimidine-5-carboxylate (30 mg, 0.06 mmol) and (1R,2R)-2-(4-methylpyrimidin-2-yl)cyclopropane-1-carboxylic acid (12 mg, 0.066 mg) in pyridine (1.5 mL) was added T₃P (305 mg, 0.48 mmol). After stirring at 40° C. for 3 h, the mixture was quenched with saturated aq·NaHCO₃ solution (10 mL) and extracted with EtOAc (3×20 mL). The combined organic layers were washed with brine (30 mL), dried over Na₂SO₄, and concentrated in vacuo to give the crude product, which was purified with Pre-TLC (DCM/MeOH=20/1) to give the product ethyl 4-((6-cyclopropyl-8-(3-methyl-2,4-dioxoimidazolidin-1-yl)imidazo[1,2-a]pyridin-2-yl)methoxy)-2-((1S,2S)-2-(4-methylpyrimidin-2-yl)cyclopropane-1-carboxamido)pyrimidine-5-carboxylate (10 mg, 26%) as a white solid. ESI-MS [M+H]+: 626.2 ¹H NMR (400 MHz, DMSO) δ 11.27 (s, 1H), 8.83 (s, 1H), 8.50 (d, J=5.1 Hz, 1H), 8.31 (d, J=1.2 Hz, 1H), 8.00 (s, 1H), 7.38 (d, J=1.4 Hz, 1H), 7.17 (d, J=5.1 Hz, 1H), 5.55 (s, 2H), 4.88 (s, 2H), 4.27-4.16 (m, 2H), 2.97 (s, 3H), 2.65-2.60 (m, 1H), 2.39 (s, 3H), 2.02-1.93 (m, 1H), 1.61 (t, J=7.4 Hz, 2H), 1.29-1.21 (m, 4H), 1.00-0.92 (m, 2H), 0.71-0.63 (m, 2H).

### Example 655

### Synthesis of (1S,2S)—N-(3-cyano-5-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (I-655); Synthesis of 3,5-diaminobenzonitrile

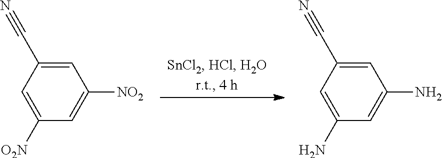 [SMILES: N#Cc1cc([N+](=O)[O-])cc([N+](=O)[O-])c1 ; "SnCl2, HCl, H2Or.t., 4 h" ; N#Cc1cc(N)cc(N)c1]

A mixture of 3,5-dinitrobenzonitrile (2 g, 10.4 mmol) and SnCl₂ (1.89 g, 10.4 mmol) in HCl (2M in water, 10 mL) was stirred at room temperature for 4 h under N₂. The reaction was quenched with water (100 mL) and extracted with EtOAc (3×100 mL). The combined organic layers were washed with brine (100 mL), dried over anhydrous Na₂SO₄, and concentrated in vacuo to give the crude product, which was purified by column chromatography (eluent: DCM/MeOH=20/1) to give 3,5-diaminobenzonitrile (280 mg, 20%) as a yellow solid. ESI-MS [M+H]+: 134.2.

### Synthesis of (1S,2S)—N-(3-amino-5-cyanophenyl)-2-(4-methylpyrimidin-2-yl)cyclopropane-1-carboxamide

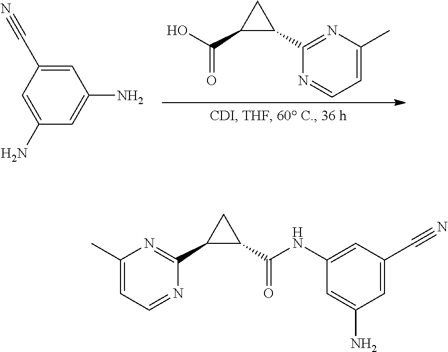 [SMILES: N#Cc1cc(N)cc(N)c1 ; Cc1ccnc([C@H]2C[C@@H]2C(=O)O)n1 ; Cc1ccnc([C@H]2C[C@@H]2C(=O)Nc2cc(N)cc(C#N)c2)n1]

A mixture of 3,5-diaminobenzonitrile (280 mg, 2.1 mmol), (1S,2S)-2-(4-methylpyrimidin-2-yl)cyclopropane-1-carboxylic acid (269 mg, 2.1 mmol) and CDI (340 mg, 2.1 mmol) in THF (10 mL) was stirred at 60° C. for 36 h under N₂. The reaction was quenched with water (50 mL) and extracted with EtOAc (3×50 mL). The combined organic layers were washed with brine (50 mL), dried over anhydrous Na₂SO₄, and concentrated in vacuo to give the crude product, which was purified by preparative TLC (eluent: DCM/MeOH=20/1) to give (1S,2S)—N-(3-amino-5-cyanophenyl)-2-(4-methylpyrimidin-2-yl)cyclopropane-1-carboxamide (300 mg, 49%) as a yellow solid. ESI-MS [M+H]⁺: 294.2.

### Synthesis of (1S,2S)—N-(3-cyano-5-(((6-cyclopropyl-8-(3-methyl-2,4-dioxoimidazolidin-1-yl) imidazo[1,2-a]pyridin-2-yl)methyl)amino)phenyl)-2-(4-methylpyrimidin-2-yl)cyclopropane-1-carboxamide (I-655)

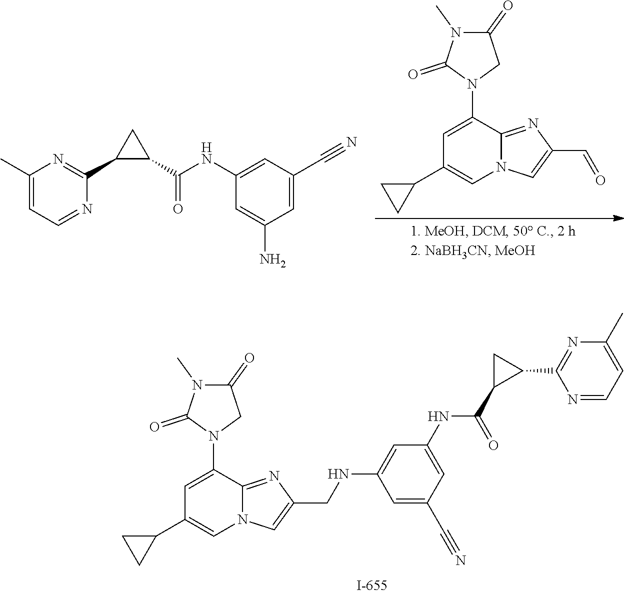 [SMILES: Cc1ccnc([C@H]2C[C@@H]2C(=O)Nc2cc(N)cc(C#N)c2)n1 ; CN1C(=O)CN(c2cc(C3CC3)cn3cc(C=O)nc23)C1=O ; Cc1ccnc([C@H]2C[C@@H]2C(=O)Nc2cc(C#N)cc(NCc3cn4cc(C5CC5)cc(N5CC(=O)N(C)C5=O)c4n3)c2)n1]

A mixture of (1S,2S)—N-(3-amino-5-cyanophenyl)-2-(4-methylpyrimidin-2-yl)cyclopropane-1-carboxamide (70 mg, 0.24 mmol), 6-cyclopropyl-8-(3-methyl-2,4-dioxoimidazolidin-1-yl)imidazo[1,2-a]pyridine-2-carbaldehyde (72.0 mg, 0.24 mmol), and Ti(O-iPr)₄ (710 mg, 2.5 mmol) in a solution of MeOH/DCM (2.0 mL/10.0 mL) was stirred at 50° C. for 2 h under N₂. After cooling to 0° C., NaBH₃CN (47 mg, 0.72 mmol) in MeOH (3.0 mL) was added to the mixture which was then stirred at room temperature for 1 h. The reaction was quenched with water (10 mL) and extracted with EtOAc (3×10 mL). The combined organic layers were washed with brine (20 mL), dried over anhydrous Na₂SO₄, then concentrated in vacuo to give the crude product, which was purified by preparative TLC (eluent: DCM/MeOH=15/1) to give (1S,2S)—N-(3-cyano-5-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide (40 mg, 29%) as a light yellow solid. ESI-MS [M+H]+: 576.2. ¹H NMR (400 MHz, DMSO) δ 10.39 (s, 1H), 8.52 (d, J=5.1 Hz, 1H), 8.25 (s, 1H), 7.71 (s, 1H), 7.33 (d, J=1.3 Hz, 1H), 7.21-7.20 (m, 2H), 7.16 (s, 1H), 6.82-6.79 (m, 1H), 6.69 (s, 1H), 4.90 (s, 2H), 4.35 (d, J=5.8 Hz, 2H), 2.97 (s, 3H), 2.55-2.52 (m, 2H), 2.41 (s, 3H), 1.95-1.91 (m, 1H), 1.52-1.46 (m, 2H), 0.96-0.91 (m, 2H), 0.66-0.62 (m, 2H).

### Example 656

### Synthesis of (1S,2S)—N-(3-(((6-cyclopropyl-8-(3-methyl-2,4-dioxoimidazolidin-1-yl)imidazo[1,2-a]pyridin-2-yl)methyl)amino)-4-fluorophenyl)-2-(4-methylpyrimidin-2-yl)cyclopropane-1-carboxamide (I-656)

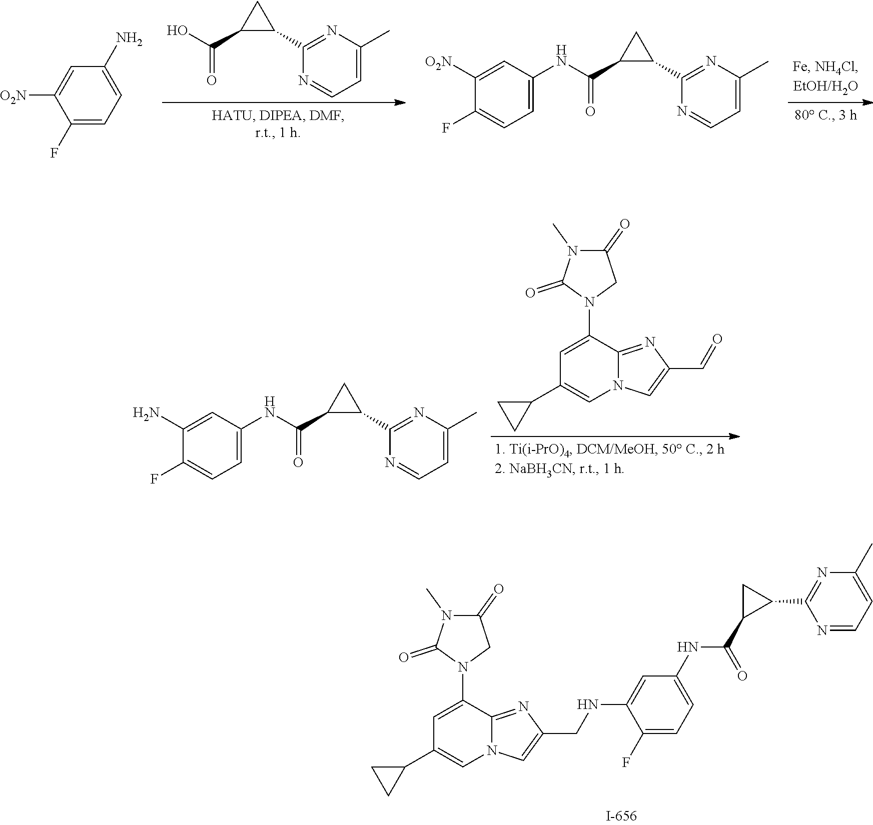

Synthesis of (1S,2S)—N-(4-fluoro-3-nitrophenyl)-2-(4-methylpyrimidin-2-yl)cyclopropane-1-carboxamide. A mixture of 4-fluoro-3-nitroaniline (262 mg, 1.68 mmol), (1S,2S)-2-(4-methylpyrimidin-2-yl)cyclopropane-1-carboxylic acid (300 mg, 1.68 mmol), HATU (958 mg, 2.52 mmol), and DIPEA (720 mg, 5.58 mmol) in DMF (15 mL) was stirred at room temperature for 1 h. The mixture was quenched with water (50 mL) and extracted with EtOAc (3×50 mL). The combined organic layers were washed with brine (50 mL), dried over Na₂SO₄ and concentrated in vacuo to give the crude product, which was purified by column chromatography (eluent: PE/EA=1/2) to give the product (1S,2S)—N-(4-fluoro-3-nitrophenyl)-2-(4-methylpyrimidin-2-yl)cyclopropane-1-carboxamide (500 mg, 93%) as a yellow solid. ESI-MS [M+H]+: 317.1

Synthesis of (1S,2S)—N-(3-amino-4-fluorophenyl)-2-(4-methylpyrimidin-2-yl)cyclopropane-1-carboxamide. To a mixture of (1S,2S)—N-(4-fluoro-3-nitrophenyl)-2-(4-methylpyrimidin-2-yl)cyclopropane-1-carboxamide (316 mg, 1.0 mmol) and NH₄Cl (64 mg, 1.2 mmol) in EtOH/H2O (10 mL/1.0 mL) was added Fe powder (336 mg, 6 mmol). The mixture was stirred at 80° C. for 3 h and then concentrated in vacuo to give the crude product, which was purified with preparative TLC (eluent: DCM/MeOH=15/1) to give the product (1S,2S)—N-(3-amino-4-fluorophenyl)-2-(4-methylpyrimidin-2-yl)cyclopropane-1-carboxamide (125 mg, 44%) as a yellow solid. ESI-MS [M+H]+: 287.1

Synthesis of (1S,2S)—N-(3-(((6-cyclopropyl-8-(3-methyl-2,4-dioxoimidazolidin-1-yl)imidazo[1,2-a]pyridin-2-yl)methyl)amino)-4-fluorophenyl)-2-(4-methylpyrimidin-2-yl)cyclopropane-1-carboxamide (I-656). To a solution of (1S,2S)—N-(3-amino-4-fluorophenyl)-2-(4-methylpyrimidin-2-yl)cyclopropane-1-carboxamide (86 mg, 0.3 mmol) and 6-cyclopropyl-8-(3-methyl-2,4-dioxoimidazolidin-1-yl)imidazo[1,2-a]pyridine-2-carbaldehyde (90 mg, 0.3 mmol) in DCM/MeOH (4 mL/4 mL) was added Ti(i-PrO)₄ (426 mg, 1.5 mmol). The mixture was stirred at 50° C. for 2 h, then after cooling to room temperature, NaBH₃CN (57 mg, 0.9 mmol) was added. Then the mixture was stirred at room temperature for 1 h. The reaction was quenched with water (20 mL) and the aqueous phase was extracted with DCM (3×30 mL). The combined organic layers were washed with brine (50 mL), dried over Na₂SO₄, concentrated in vacuo to give the crude product, which was purified by preparative HPLC to give the product (1S,2S)—N-(3-(((6-cyclopropyl-8-(3-methyl-2,4-dioxoimidazolidin-1-yl)imidazo[1,2-a]pyridin-2-yl)methyl)amino)-4-fluorophenyl)-2-(4-methylpyrimidin-2-yl)cyclopropane-1-carboxamide (66 mg, 39%) as a yellow solid. ESI-MS [M+H]+: 569.2 ¹H NMR (400 MHz, DMSO) δ 10.08 (s, 1H), 8.50 (d, J=5.1 Hz, 1H), 8.23 (d, J=1.0 Hz, 1H), 7.66 (s, 1H), 7.33 (d, J=1.4 Hz, 1H), 7.18 (d, J=5.1 Hz, 1H), 7.00 (dd, J=8.2, 2.2 Hz, 1H), 6.98-6.87 (m, 1H), 6.86-6.77 (m, 1H), 6.07 (t, J=5.1 Hz, 1H), 4.92 (s, 2H), 4.37 (d, J=5.9 Hz, 2H), 2.97 (s, 3H), 2.46-2.42 (m, 1H), 2.40 (s, 3H), 2.35-2.30 (m, 1H), 1.96-1.89 (m, 1H), 1.48-1.40 (m, 2H), 0.95-0.88 (m, 2H), 0.68-0.59 (m, 2H).

### Example 657

### Synthesis of (1S,2S)—N-(3-((6-cyclopropyl-8-(3-methyl-2,4-dioxoimidazolidin-1-yl)imidazo[1,2-a]pyridin-2-yl)methoxy)-4-fluorophenyl)-2-(4-methylpyrimidin-2-yl)cyclopropane-1-carboxamide (I-657)

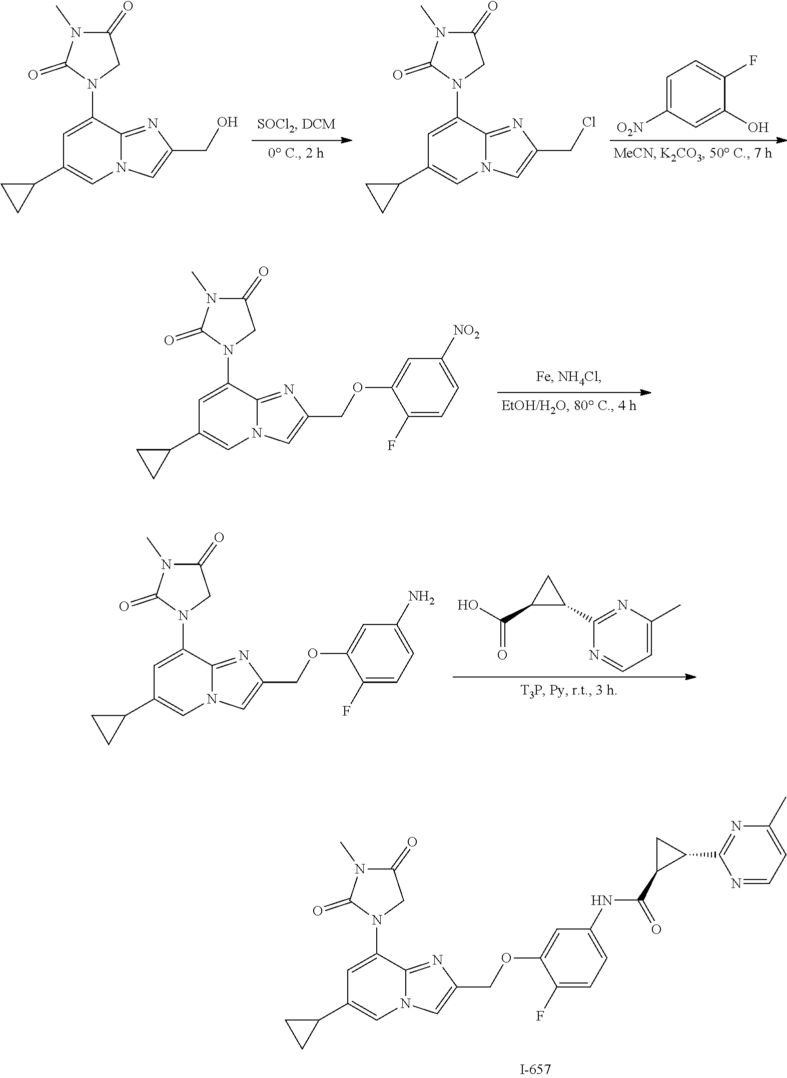

Synthesis of 1-(2-(chloromethyl)-6-cyclopropylimidazo[1,2-a]pyridin-8-yl)-3-methyl imidazolidine-2,4-dione. To a mixture of 1-(6-cyclopropyl-2-(hydroxymethyl)imidazo[1,2-a]pyridin-8-yl)-3-methylimidazolidine-2,4-dione (100 mg, 0.33 mmol) in DCM (4 mL) was added SOCl₂ at 0° C. and the mixture was stirred at 0° C. for 2 h. The mixture was concentrated in vacuo to give the crude, which was purified by preparative TLC (eluent: DCM/MeOH=20/1) to give the product (42 mg, 40%) as yellow solid. ESI-MS [M+H]+: 319.1.

Synthesis of 1-(6-cyclopropyl-2-((2-fluoro-5-nitrophenoxy)methyl)imidazo[1,2-a]pyridin-8-yl)-3-methylimidazolidine-2,4-dione. A mixture of 1-(2-(chloromethyl)-6-cyclopropylimidazo[1,2-a]pyridin-8-yl)-3-methylimidazolidine-2,4-dione (100 mg, 0.32 mmol), 2-fluoro-5-nitrophenol (101 mg, 0.64 mmol), and K₂CO₃ (443 mg, 3.20 mmol) in MeCN (5 mL) was stirred at 50° C. under N₂ for 7 h. The reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC (eluent: PE/EA=2/1) to give 1-(6-cyclopropyl-2-((2-fluoro-5-nitrophenoxy)methyl)imidazo[1,2-a]pyridin-8-yl)-3-methylimidazolidine-2,4-dione (105 mg, 75%) as yellow solid. ESI-MS [M+H]+: 440.1.

Synthesis of 1-(2-((5-amino-2-fluorophenoxy)methyl)-6-cyclopropylimidazo[1,2-a]pyridin-8-yl)-3-methylimidazolidine-2,4-dione. A mixture of 1-(6-cyclopropyl-2-((2-fluoro-5-nitrophenoxy)methyl)imidazo[1,2-a]pyridin-8-yl)-3-methylimidazolidine-2,4-dione (24 mg, 0.06 mmol), NH₄Cl (3.5 mg, 0.07 mmol), and Fe powder (18.4 mg, 0.33 mmol) in EtOH (2 mL) and H2O (0.2 mL) was stirred at 80° C. for 4 h under N₂. The reaction mixture was filtered and the filtrate was concentrated in vacuo to give the crude, which was purified by preparative TLC (eluent: DCM/MeOH=20/1) to give 1-(2-((5-amino-2-fluorophenoxy)methyl)-6-cyclopropylimidazo[1,2-a] pyridin-8-yl)-3-methylimidazolidine-2,4-dione (13 mg, 53%) as yellow solid. ESI-MS [M+H]+: 410.1.

Synthesis of (1S,2S)—N-(3-((6-cyclopropyl-8-(3-methyl-2,4-dioxoimidazolidin-1-yl)imidazo[1,2-a]pyridin-2-yl)methoxy)-4-fluorophenyl)-2-(4-methylpyrimidin-2-yl)cyclopropane-1-carboxamide (I-657). To a mixture of 1-(2-((5-amino-2-fluorophenoxy)methyl)-6-cyclopropylimidazo[1,2-a]pyridin-8-yl)-3-methylimidazolidine-2,4-dione (62 mg, 0.15 mmol) and (1S,2S)-2-(4-methylpyrimidin-2-yl)cyclopropane-1-carboxylic acid (62 mg, 0.15 mmol)) in pyridine (0.5 mL) was added T₃P (734 mg, 15 mmol). After stirring at room temperature for 3 h, the reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC (eluent: DCM/MeOH=20/1) to give (1S,2S)—N-(3-((6-cyclopropyl-8-(3-methyl-2,4-dioxoimidazolidin-1-yl)imidazo[1,2-a]pyridin-2-yl)methoxy)-4-fluorophenyl)-2-(4-methylpyrimidin-2-yl)cyclopropane-1-carboxamide (70 mg, 82%) as a yellow solid. ESI-MS [M+H]+: 570.2, ¹H NMR (400 MHz, DMSO) δ 10.36 (s, 1H), 8.53 (d, J=5.1 Hz, 1H), 8.30 (d, J=1.1 Hz, 1H), 7.96 (s, 1H), 7.75-7.73 (m, 1H), 7.39 (d, J=1.5 Hz, 1H), 7.21 (d, J=5.1 Hz, 1H), 7.16-7.10 (m, 1H), 7.09-7.05 (m, 1H), 5.21 (s, 2H), 4.86 (s, 2H), 2.97 (s, 3H), 2.54 (s, 1H), 2.42 (s, 3H), 2.39-2.37 (m, 1H), 2.00-1.93 (m, 1H), 1.54-1.46 (m, 2H), 1.00-0.90 (m, 2H), 0.71-0.60 (m, 2H).

### Example 658

### Synthesis of (1S,2S)—N-(4-chloro-3-(((6-cyclopropyl-8-(3-methyl-2,4-dioxoimidazolidin-1-yl)imidazo[1,2-a]pyridin-2-yl)methyl)amino)-5-fluorophenyl)-2-(4-methylpyrimidin-2-yl)cyclopropane-1-carboxamide (I-658)

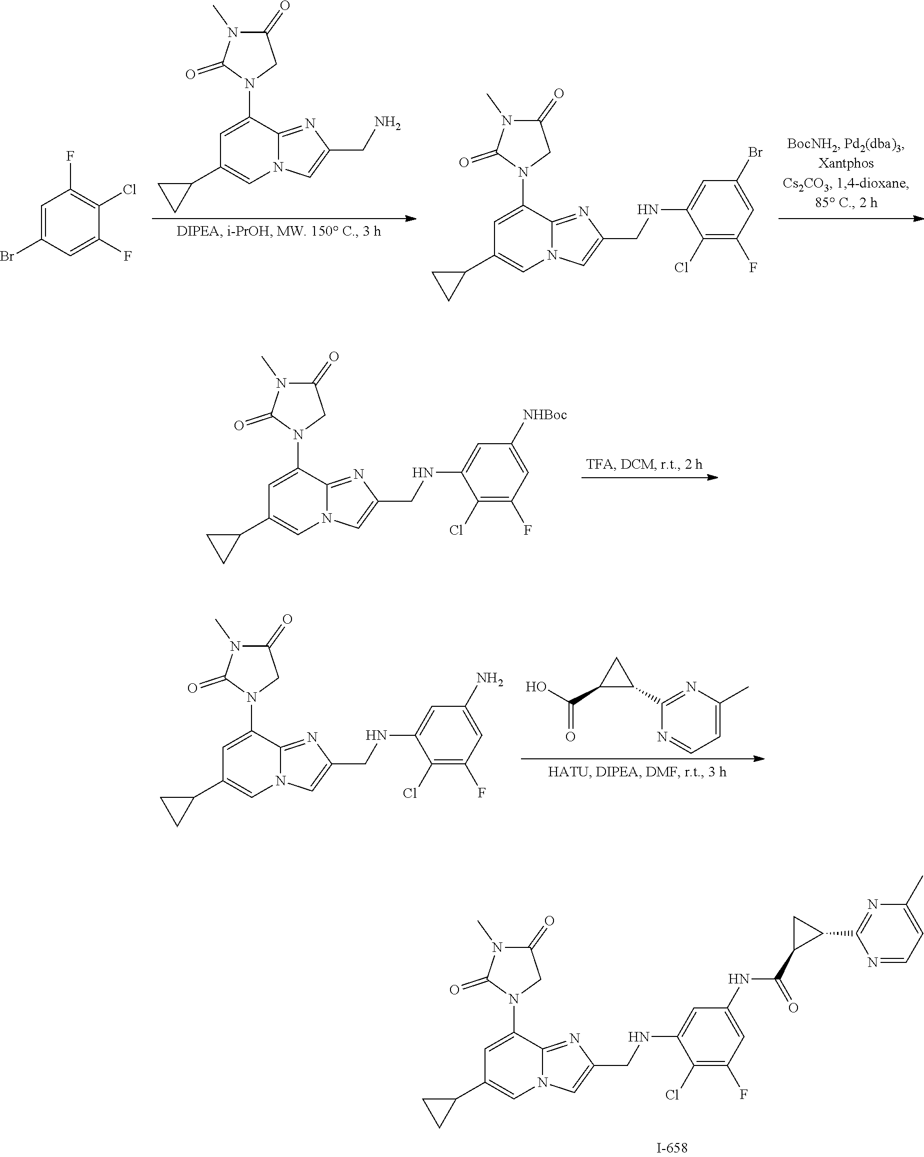

Synthesis of 1-(2-(((5-bromo-2-chloro-3-fluorophenyl)amino)methyl)-6-cyclopropylimidazo[1,2-a]pyridin-8-yl)-3-methylimidazolidine-2,4-dione. A solution of 5-bromo-2-chloro-1,3-difluorobenzene (648 mg, 2.34 mmol), 1-(2-(aminomethyl)-6-cyclopropylimidazo[1,2-a]pyridin-8-yl)-3-methylimidazolidine-2,4-dione (200 mg, 0.67 mmol) and DIPEA (260 mg, 0.21 mmol) in i-PrOH (2 mL) was stirred in a sealed tube. After degassing with N₂ for 1 min, the reaction was irradiated in a microwave reactor at 150° C. for 3 h. The reaction was concentrated in vacuo to give the crude product, which was purified by preparative TLC (eluent: DCM/MeOH=15/1) to give 1-(2-(((5-bromo-2-chloro-3-fluorophenyl)amino)methyl)-6-cyclopropylimidazo[1,2-a]pyridin-8-yl)-3-methylimidazolidine-2,4-dione (170 mg, 50%) as a yellow oil. ESI-MS [M+H]+: 506.0

Synthesis of tert-butyl (4-chloro-3-(((6-cyclopropyl-8-(3-methyl-2,4-dioxoimidazolidin-1-yl)imidazo[1,2-a]pyridin-2-yl)methyl)amino)-5-fluorophenyl)carbamate. To a solution of 1-(2-(((5-bromo-2-chloro-3-fluorophenyl)amino)methyl)-6-cyclopropylimidazo[1,2-a]pyridin-8-yl)-3-methylimidazolidine-2,4-dione (170 mg, 0.34 mmol), tert-butyl carbamate (48 mg, 0.41 mmol), and Cs₂CO₃ (222 mg, 0.68 mmol) in toluene (10 mL) were added Pd₂(dba)₃ (62 mg, 0.068 mmol) and Xantphos (59 mg, 0.10 mmol). The reaction mixture was stirred at 85° C. for 2 h under N₂. The reaction mixture was filtered through Celite® and the filter cake was washed with DCM/MeOH (20/1, 30 mL). The filtrate was concentrated in vacuo to give the crude, which was purified by preparative TLC (eluent: PE/EtOAc=2/1) to give tert-butyl (4-chloro-3-(((6-cyclopropyl-8-(3-methyl-2,4-dioxoimidazolidin-1-yl)imidazo[1,2-a]pyridin-2-yl)methyl)amino)-5-fluorophenyl)carbamate (160 mg, 87%) as a yellow solid. ESI-MS [M+H]+: 543.2

Synthesis of 1-(2-(((5-amino-2-chloro-3-fluorophenyl)amino)methyl)-6-cyclopropylimidazo[1,2-a]pyridin-8-yl)-3-methylimidazolidine-2,4-dione. To a mixture of tert-butyl (4-chloro-3-(((6-cyclopropyl-8-(3-methyl-2,4-dioxoimidazolidin-1-yl)imidazo[1,2-a]pyridin-2-yl)methyl)amino)-5-fluorophenyl)carbamate (160 mg, 0.30 mmol) in DCM (3 mL) was added TFA (1 ml) at room temperature. The resulting reaction was stirred at room temperature for 2 h. The reaction was diluted with saturated aq·NaHCO₃ solution (5 mL) and extracted with DCM/MeOH (20/1, 3×30 mL). The combined organic layers were washed with brine (20 mL), dried over anhydrous Na₂SO₄, and concentrated in vacuo to give the crude product, which was purified by preparative TLC (eluent: DCM/MeOH=15/1) to give 1-(2-(((5-amino-2-chloro-3-fluorophenyl)amino)methyl)-6-cyclopropylimidazo[1,2-a]pyridin-8-yl)-3-methylimidazolidine-2,4-dione (130 mg, quant) as a yellow solid. ESI-MS [M+H]+: 443.1.

Synthesis of (1S,2S)—N-(4-chloro-3-(((6-cyclopropyl-8-(3-methyl-2,4-dioxoimidazolidin-1-yl)imidazo[1,2-a]pyridin-2-yl)methyl)amino)-5-fluorophenyl)-2-(4-methylpyrimidin-2-yl)cyclopropane-1-carboxamide (I-658). A mixture of 1-(2-(((5-amino-2-chloro-3-fluorophenyl)amino)methyl)-6-cyclopropylimidazo[1,2-a]pyridin-8-yl)-3-methylimidazolidine-2,4-dione (130 mg, 0.29 mmol), (1S,2S)-2-(4-methylpyrimidin-2-yl)cyclopropane-1-carboxylic acid (52 mg, 0.29 mmol), HATU (165 mg, 0.44 mmol) and DIPEA (187 mg, 1.45 mmol) in DMF (2 mL) was stirred at room temperature for 3 h. Water (20 mL) was added and the mixture was extracted with DCM/MeOH (20/1, 3×30 mL). The combined organic layers were washed with brine (20 mL), dried over Na₂SO₄, filtered and concentrated in vacuo to give the crude, which was purified by preparative TLC (eluent: DCM/MeOH=20/1) to give (1S,2S)—N-(4-chloro-3-(((6-cyclopropyl-8-(3-methyl-2,4-dioxoimidazolidin-1-yl)imidazo[1,2-a]pyridin-2-yl)methyl)amino)-5-fluorophenyl)-2-(4-methylpyrimidin-2-yl)cyclopropane-1-carboxamide (35 mg, 20%) as a white solid. ESI-MS [M+H]⁺: 603.2. ¹H NMR (400 MHz, DMSO) δ 10.34 (s, 1H), 8.50 (d, J=5.1 Hz, 1H), 8.23 (s, 1H), 7.64 (s, 1H), 7.34 (s, 1H), 7.19 (d, J=5.1 Hz, 1H), 7.04 (d, J=10.1 Hz, 1H), 6.73 (s, 1H), 6.38 (t, J=5.8 Hz, 1H), 4.91 (s, 2H), 4.44 (d, J=5.6 Hz, 2H), 2.97 (s, 3H), 2.49-2.43 (m, 1H), 2.39 (s, 3H), 2.34 (m, 1H), 1.97-1.88 (m, 1H), 1.50-1.40 (m, 2H), 0.97-0.88 (m, 2H), 0.67-0.60 (m, 2H).

### Example 659

### Synthesis of (1S,2S)—N-(5-(((6-cyclopropyl-8-(3-methyl-2,4-dioxoimidazolidin-1-yl)imidazo[1,2-a]pyridin-2-yl)methyl)amino)-2,4-difluorophenyl)-2-(4-methylpyrimidin-2-yl)cyclopropane-1-carboxamide (I-659)

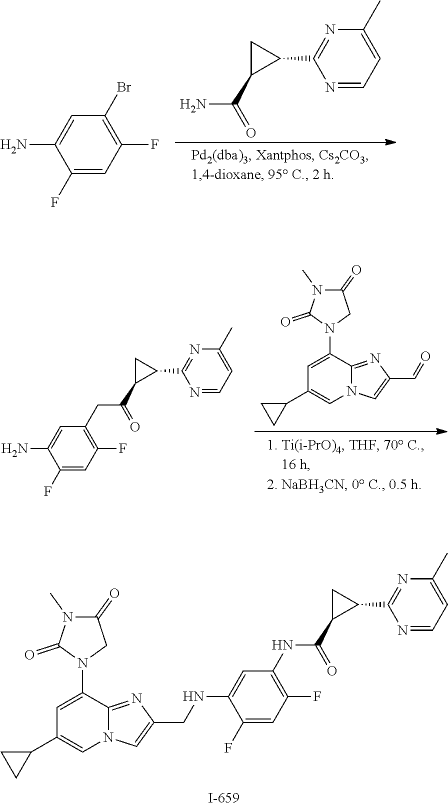 [SMILES: Nc1cc(Br)c(F)cc1F ; Cc1ccnc([C@H]2C[C@@H]2C(N)=O)n1 ; Cc1ccnc([C@H]2C[C@@H]2C(=O)Cc2cc(N)c(F)cc2F)n1 ; CN1C(=O)CN(c2cc(C3CC3)cn3cc(C=O)nc23)C1=O ; Cc1ccnc([C@H]2C[C@@H]2C(=O)Nc2cc(NCc3cn4cc(C5CC5)cc(N5CC(=O)N(C)C5=O)c4n3)c(F)cc2F)n1]

Synthesis of (1S,2S)—N-(5-amino-2,4-difluorophenyl)-2-(4-methylpyrimidin-2-yl)cyclopropane-1-carboxamide. To a mixture of 5-bromo-2,4-difluoroaniline (141.2 mg, 0.68 mmol), (1S,2S)-2-(4-methylpyrimidin-2-yl)cyclopropane-1-carboxamide (60 mg, 0.68 mmol) and Cs₂CO₃ (665 mg, 2.04 mmol) in 1,4-dioxane (14 mL) were added Pd₂(dba)₃ (124.5 mg, 0.136 mmol) and XantPhos (157.4 mg, 0.272 mmol) at 25° C. After stirring at 95° C. for 2 h under N₂, the reaction mixture was filtered through Celite© and the filter cake was washed with DCM/MeOH (10/1, 50 mL). The filtrate was concentrated in vacuo to give the crude, which was purified by column chromatography (eluent: DCM/MeOH=30/1˜10/1) to give (1S,2S)—N-(5-amino-2,4-difluorophenyl)-2-(4-methylpyrimidin-2-yl)cyclopropane-1-carboxamide (20 mg, 10%) as a yellow solid. ESI-MS [M+H]+: 305.1

Synthesis of (1S,2S)—N-(5-(((6-cyclopropyl-8-(3-methyl-2,4-dioxoimidazolidin-1-yl)imidazo[1,2-a]pyridin-2-yl)methyl)amino)-2,4-difluorophenyl)-2-(4-methylpyrimidin-2-yl)cyclopropane-1-carboxamide (I-659). To a mixture of (1S,2S)—N-(5-amino-2,4-difluorophenyl)-2-(4-methylpyrimidin-2-yl)cyclopropane-1-carboxamide (20 mg, 0.065 mmol) and 6-cyclopropyl-8-(3-methyl-2,4-dioxoimidazolidin-1-yl)imidazo[1,2-a]pyridine-2-carbaldehyde (30 mg, 0.098 mmol) in THF (3 mL) was added Ti(i-PrO)₄ (93 mg, 0.325 mmol). The resulting mixture was stirred at 70° C. for 16 h under N₂. After cooling to 25° C., NaBH₃CN (14.5 mg, 0.23 mmol) was added and the mixture was stirred at 25° C. for 0.5 h. Then the mixture was quenched with water (20 mL) and extracted with EtOAc (3×10 mL). The organic were concentrated in vacuo to give the crude, which was purified by column chromatography (eluent: DCM/MeOH=30/1˜10/1) to give (1S,2S)—N-(5-(((6-cyclopropyl-8-(3-methyl-2,4-dioxoimidazolidin-1-yl)imidazo[1,2-a]pyridin-2-yl)methyl)amino)-2,4-difluorophenyl)-2-(4-methylpyrimidin-2-yl)cyclopropane-1-carboxamide (20 mg, 53%) as a white solid. ESI-MS [M+H]+: 587.2. ¹H NMR (400 MHz, DMSO) δ 9.86 (s, 1H), 8.51 (d, J=5.1 Hz, 1H), 8.23 (d, J=1.2 Hz, 1H), 7.69 (s, 1H), 7.34 (d, J=1.5 Hz, 1H), 7.23-7.11 (m, 3H), 5.93-5.91 (m, 1H), 4.92 (s, 2H), 4.35 (d, J=5.9 Hz, 2H), 2.97 (s, 3H), 2.47-2.43 (m, 2H), 2.40 (s, 3H), 1.97-1.90 (m, 1H), 1.47-1.44 (m, 2H), 0.96-0.91 (m, 2H), 0.66-0.62 (m, 2H).

### Example 660

### Synthesis of (1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide (I-660)

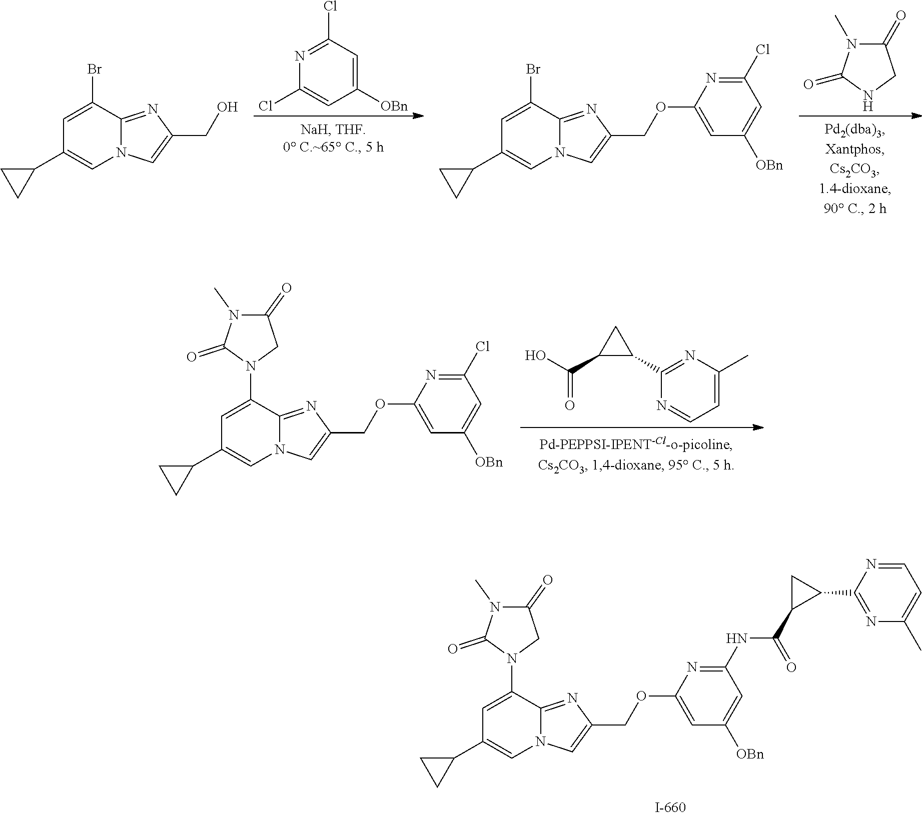

Synthesis of 2-(((4-(benzyloxy)-6-chloropyridin-2-yl)oxy)methyl)-8-bromo-6-cyclopropylimidazo[1,2-a]pyridine. To a mixture of (8-bromo-6-cyclopropylimidazo[1,2-a]pyridin-2-yl)methanol (100 mg, 0.38 mmol) in dry THF (10.0 mL) was added NaH (60 mg, 1.5 mmol, 60% dispersion in mineral oil) at 0° C. After stirring the mixture at 0° C. for 0.5 h, a solution of 4-(benzyloxy)-2,6-dichloropyridine (95 mg, 1.2 mmol) in dry THF (2.0 mL) was added and stirred at 65° C. for another 5 h. The reaction was quenched with saturated aq·NH₄Cl (20 mL) and extracted with EtOAc (3×50 mL). The combined organic layers were washed with brine (50 mL), dried over anhydrous Na₂SO₄, and concentrated in vacuo to give the crude, which was purified by silica gel column chromatography (eluent: DCM/MeOH=50/1) to give 2-(((4-(benzyloxy)-6-chloropyridin-2-yl)oxy)methyl)-8-bromo-6-cyclopropylimidazo[1,2-a]pyridine (110 mg, 60%) as a yellow solid. ESI-MS [M+H]⁺: 486.2.

Synthesis of 1-(2-(((4-(benzyloxy)-6-chloropyridin-2-yl)oxy)methyl)-6-cyclopropylimidazo[1,2-a]pyridin-8-yl)-3-methylimidazolidine-2,4-dione. A mixture of 2-(((4-(benzyloxy)-6-chloropyridin-2-yl)oxy)methyl)-8-bromo-6-cyclopropylimidazo[1,2-a]pyridine (110 mg, 0.23 mmol), 3-methylimidazolidine-2,4-dione (78 mg, 0.25 mmol), and Cs₂CO₃ (222 mg, 0.68 mmol) in 1,4-dioxane (8 mL) were added Pd₂(dba)₃ (21 mg, 0.023 mmol) and Xantphos (26 mg, 0.045 mmol). After stirring at 85° C. for 8 h under N₂, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=30/1) to give 1-(2-(((4-(benzyloxy)-6-chloropyridin-2-yl)oxy)methyl)-6-cyclopropylimidazo[1,2-a]pyridin-8-yl)-3-methylimidazolidine-2,4-dione (60 mg, 51%) as a yellow solid. ESI-MS [M+H]⁺: 518.2.

Synthesis of (1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide (I-660). To a mixture of 1-(2-(((4-(benzyloxy)-6-chloropyridin-2-yl)oxy)methyl)-6-cyclopropylimidazo[1,2-a]pyridin-8-yl)-3-methylimidazolidine-2,4-dione (60 mg, 0.12 mmol), (1S,2S)-2-(4-methylpyrimidin-2-yl)cyclopropane-1-carboxamide (23 mg, 0.13 mmol), and Cs₂CO₃ (113 mg, 0.35 mmol) in 1,4-dioxane (5 mL) was added Pd-PEPPSI-IPENT-Cl-o-picoline (10 mg, 0.012 mmol). The reaction mixture was stirred at 90° C. for 2 h under N₂. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=20/1) to give (1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide (42 mg, 55%) as a white solid. ESI-MS [M+H]+: 659.2. ¹H NMR (400 MHz, DMSO) δ 10.66 (s, 1H), 8.53 (d, J=5.1 Hz, 1H), 8.27 (s, 1H), 7.92 (s, 1H), 7.52-7.27 (m, 7H), 7.21 (d, J=5.1 Hz, 1H), 6.20 (d, J=1.8 Hz, 1H), 5.38 (s, 2H), 5.14 (s, 2H), 4.88 (s, 2H), 2.97 (s, 3H), 2.56-2.52 (m, 2H), 2.42 (s, 3H), 1.98-1.92 (m, 1H), 1.55-1.52 (m, 2H), 0.97-0.92 (m, 2H), 0.67-0.64 (m, 2H).

### Example 661

### Synthesis of (1S,2S)—N-(6-((6-cyclopropyl-8-(3-methyl-2,4-dioxoimidazolidin-1-yl)imidazo[1,2-a]pyridin-2-yl)methoxy)-4-hydroxypyridin-2-yl)-2-(4-methylpyrimidin-2-yl)cyclopropane-1-carboxamide (I-661)

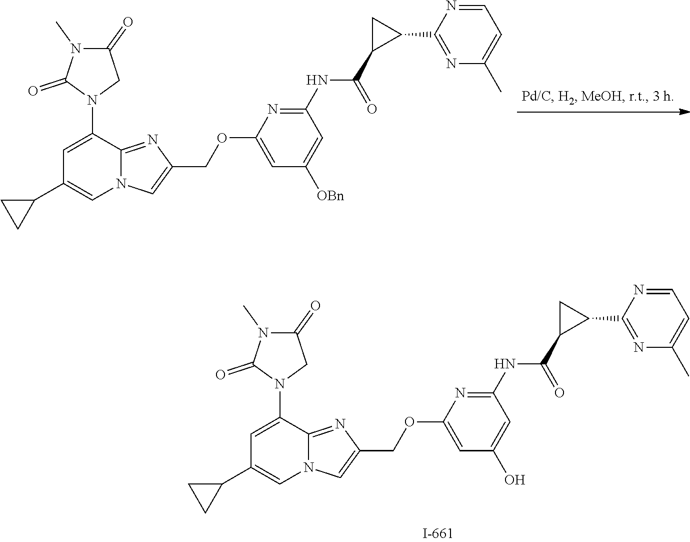 [SMILES: Cc1ccnc([C@H]2C[C@@H]2C(=O)Nc2cc(OCc3ccccc3)cc(OCc3cn4cc(C5CC5)cc(N5CC(=O)N(C)C5=O)c4n3)n2)n1 ; Cc1ccnc([C@H]2C[C@@H]2C(=O)Nc2cc(O)cc(OCc3cn4cc(C5CC5)cc(N5CC(=O)N(C)C5=O)c4n3)n2)n1]

A mixture of (1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide (100 mg, 0.15 mmol) and Pd/C (10%, 20 mg) in MeOH (5 mL) was stirred at room temperature for 3 h under H₂. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=15/1) to give (1S,2S)—N-(6-((6-cyclopropyl-8-(3-methyl-2,4-dioxoimidazolidin-1-yl)imidazo-[1,2-a]pyridin-2-yl)methoxy)-4-hydroxypyridin-2-yl)-2-(4-methylpyrimidin-2-yl)cyclopropane-1-carboxamide (75 mg, 88%) as a white solid. ESI-MS [M+H]⁺: 569.2. ¹H NMR (400 MHz, DMSO) δ 10.52 (d, J=6.6 Hz, 2H), 8.53 (d, J=4.7 Hz, 1H), 8.26 (s, 1H), 7.90 (s, 1H), 7.35 (s, 1H), 7.27 (s, 1H), 7.21 (d, J=4.9 Hz, 1H), 5.87 (s, 1H), 5.34 (s, 2H), 4.88 (s, 2H), 2.97 (s, 3H), 2.56-2.52 (m, 2H), 2.42 (s, 3H), 2.00-1.87 (m, 1H), 1.55-1.50 (m, 2H), 1.04-0.85 (m, 2H), 0.67-0.63 (m, 2H).

### Example 662

### Synthesis of (1S,2S)—N-(6-((6-cyclopropyl-8-(3-methyl-2,4-dioxoimidazolidin-1-yl)imidazo[1,2-a]pyridin-2-yl)methoxy)-2-(1-hydroxyethyl)pyrimidin-4-yl)-2-(4-methylpyrimidin-2-yl)cyclopropane-1-carboxamide (I-662)

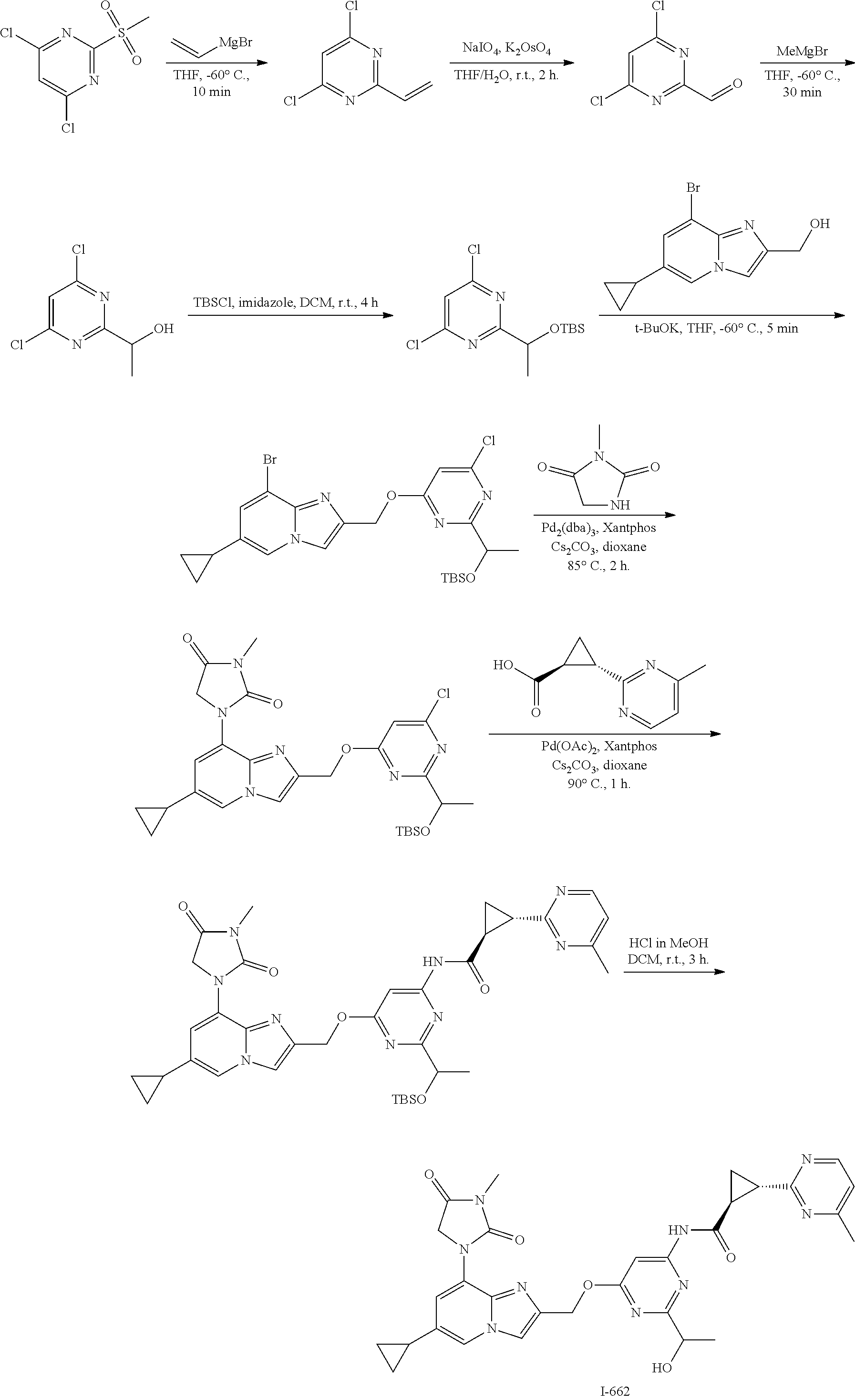

Synthesis of 4,6-dichloro-2-vinylpyrimidine. To a mixture of 4,6-dichloro-2-(methylsulfonyl)pyrimidine (50 g, 221 mmol) in THF (300 mL) was added vinylmagnesium bromide in THF (1M solution in THF, 332 mL, 332 mmol) dropwise at −60° C. under N₂. After stirring at −60° C. for 10 min, the reaction was quenched with saturated aq·NH₄Cl (100 mL) and extracted with EtOAc (2×200 mL). The combined organic layers were washed with brine (80 mL), dried over anhydrous Na₂SO₄, and concentrated in vacuo to give the crude, which was purified by silica gel column chromatography (eluent: PE/EtOAc=15/1) to give 4,6-dichloro-2-vinylpyrimidine (35 g, 91%) as yellow oil. ESI-MS [M+H]+: No MS.

Synthesis of 4,6-dichloropyrimidine-2-carbaldehyde. To a mixture of 4,6-dichloro-2-vinylpyrimidine (15 g, 86.2 mmol) in THF (200 mL) and water (100 mL) were added NaIO₄ (55.3 g, 258.6 mmol) and K₂OsO₄2H₂O (1.59 g, 4.3 mmol) at 0° C. After stirring at room temperature for 2 h, the reaction mixture was filtered through Celite© and the filter cake was washed with EtOAc (200 mL). The filtrate was concentrated in vacuo to give the crude, which was purified with silica gel column chromatography (eluent: PE/EtOAc=10/1) to give 4,6-dichloropyrimidine-2-carbaldehyde (12 g, 79%) as yellow oil. ESI-MS [M+H]+: 177.0.

Synthesis of 1-(4,6-dichloropyrimidin-2-yl)ethan-1-ol. To a solution of 4,6-dichloropyrimidine-2-carbaldehyde (5 g, 28.4 mmol) in THF (50 mL) was added MeMgBr in THF (3M solution in THF, 19 mL, 56.8 mmol) at −60° C. dropwise. After stirring at −60° C. for 30 min under N₂, the reaction was quenched with saturated aqueous NH₄Cl (50 mL), extracted with EtOAc (3×50 mL). The combined organic layers were washed with brine (60 mL), dried over anhydrous Na₂SO₄, and concentrated in vacuo to give the crude, which was purified by silica gel column chromatography (eluent: PE/EtOAc=5/1) to give 1-(4,6-dichloropyrimidin-2-yl)ethan-1-ol (500 mg, 9%) as a yellow oil. ESI-MS [M+H]+: 193.0.

Synthesis of 2-(1-((tert-butyldimethylsilyl)oxy)ethyl)-4,6-dichloropyrimidine. To a mixture of 1-(4,6-dichloropyrimidin-2-yl)ethan-1-ol (500 mg, 2.6 mmol) and imidazole (265 mg, 3.9 mmol) in DCM (10 mL) was added TBSCl (465 mg, 3.1 mmol). After stirring at room temperature for 4 h under N₂, the reaction was diluted with DCM (50 mL) and washed with water (40 mL). The organic layer was washed with brine (40 mL), dried over anhydrous Na₂SO₄, and concentrated in vacuo to give the crude, which was purified by preparative TLC (eluent: PE/EtOAc=50/1) give 2-(1-((tert-butyldimethylsilyl)oxy)ethyl)-4,6-dichloropyrimidine (300 mg, 38%) as a yellow oil. ESI-MS [M+H]+: 307.0.

Synthesis of 8-bromo-2-(((2-(1-((tert-butyldimethylsilyl)oxy)ethyl)-6-chloropyrimidin-4-yl)oxy)methyl)-6-cyclopropylimidazo[1,2-a]pyridine. To a mixture of (8-bromo-6-cyclopropylimidazo[1,2-a]pyridin-2-yl)methanol (230 mg, 0.88 mmol), 2-(1-((tert-butyldimethylsilyl)oxy)ethyl)-4,6-dichloropyrimidine (270 mg, 0.88 mmol) in THF (5 mL) was added t-BuOK (1M in THF, 0.88 mL, 0.88 mmol) dropwise at −60° C. After stirring at −60° C. for 5 min under N₂, the reaction was quenched with saturated aq·NH₄Cl (30 mL) and extracted with EtOAc (3×30 mL). The combined organic layers were washed with brine (30 mL), dried over anhydrous Na₂SO₄, and concentrated in vacuo to give the crude product, which was purified by preparative TLC (eluent: PE/EtOAc=4/1) to give 8-bromo-2-(((2-(1-((tert-butyldimethylsilyl)oxy)ethyl)-6-chloropyrimidin-4-yl)oxy)methyl)-6-cyclopropylimidazo[1,2-a]pyridine (230 mg, 50%) as a yellow oil. ESI-MS [M+H]+: 537.1.

Synthesis of 1-(2-(((2-(1-((tert-butyldimethylsilyl)oxy)ethyl)-6-chloropyrimidin-4-yl)oxy)methyl)-6-cyclopropylimidazo[1,2-a]pyridin-8-yl)-3-methylimidazolidine-2,4-dione. To a mixture of 8-bromo-2-(((2-(1-((tert-butyldimethylsilyl)oxy)ethyl)-6-chloropyrimidin-4-yl)oxy)methyl)-6-cyclopropylimidazo[1,2-a]pyridine (230 mg, 0.43 mmol), 3-methylimidazolidine-2,4-dione (49 mg, 0.43 mmol), and Cs₂CO₃ (421 mg, 1.29 mmol) in 1, 4-dioxane (20 mL) were added Pd₂(dba)₃ (39 mg, 0.043 mmol) and Xantphos (50 mg, 0.086 mmol). The resulting reaction mixture was stirred at 85° C. for 2 h under N₂. The reaction mixture was filtered through Celite® and the filter cake was washed with DCM/MeOH (10/1, 50 mL). The filtrate was concentrated in vacuo to give the crude, which was purified by silica gel column chromatography (eluent: PE/EtOAc=2/1) to give 1-(2-(((2-(1-((tert-butyldimethylsilyl)oxy)ethyl)-6-chloropyrimidin-4-yl)oxy)methyl)-6-cyclopropylimidazo[1,2-a]pyridin-8-yl)-3-methylimidazolidine-2,4-dione (80 mg, 33%) as a yellow solid. ESI-MS [M+H]+: 571.2.

Synthesis of (1S,2S)—N-(2-(1-((tert-butyldimethylsilyl)oxy)ethyl)-6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide. To a mixture of 1-(2-(((2-(1-((tert-butyldimethylsilyl)oxy)ethyl)-6-chloropyrimidin-4-yl)oxy)methyl)-6-cyclopropylimidazo[1,2-a]pyridin-8-yl)-3-methylimidazolidine-2,4-dione (80 mg, 0.14 mmol), (1S,2S)-2-(4-methylpyrimidin-2-yl)cyclopropane-1-carboxamide (37 mg, 0.21 mmol), and Cs₂CO₃ (137 mg, 0.42 mmol) in 1,4-dioxane (5 mL) were added Pd(OAc)₂ (6 mg, 0.028 mmol) and Xantphos (32 mg, 0.056 mmol). After stirring at 90° C. for 1 h under N₂, the reaction mixture was filtered through Celite® and the filter cake was washed with DCM/MeOH (10/1, 50 mL). The filtrate was concentrated in vacuo to give the crude, which was purified by silica gel column chromatography (eluent: DCM/MeOH=20/1) to give (1S,2S)—N-(2-(1-((tert-butyldimethylsilyl)oxy)ethyl)-6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (50 mg, 50%) as a yellow solid. ESI-MS [M+H]+: 712.3

Synthesis of (1S,2S)—N-(6-((6-cyclopropyl-8-(3-methyl-2,4-dioxoimidazolidin-1-yl)imidazo[1,2-a]pyridin-2-yl)methoxy)-2-(1-hydroxyethyl)pyrimidin-4-yl)-2-(4-methylpyrimidin-2-yl)cyclopropane-1-carboxamide (I-662). To a solution of (1S,2S)—N-(2-(1-((tert-butyldimethylsilyl)oxy)ethyl)-6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide (50 mg, 0.07 mmol) in DCM (5 mL) was added HCl (4M solution in MeOH, 1 mL). After stirring at room temperature for 3 h, the reaction was concentrated in vacuo to give the crude, which was purified by preparative TLC (eluent: DCM/MeOH=15/1) to give (1S,2S)—N-(6-((6-cyclopropyl-8-(3-methyl-2,4-dioxoimidazolidin-1-yl)imidazo[1,2-a]pyridin-2-yl)methoxy)-2-(1-hydroxyethyl)pyrimidin-4-yl)-2-(4-methylpyrimidin-2-yl)cyclopropane-1-carboxamide (30 mg, 71%) as a yellow solid. ESI-MS [M+H]⁺: 598.3. ¹H NMR (400 MHz, DMSO) δ 11.25 (d, J=1.7 Hz, 1H), 8.53 (d, J=5.1 Hz, 1H), 8.28 (d, J=1.3 Hz, 1H), 7.98 (s, 1H), 7.40-7.33 (m, 2H), 7.21 (d, J=5.1 Hz, 1H), 5.52 (s, 2H), 5.09 (m, 1H), 4.89 (s, 2H), 4.65-4.58 (m, 1H), 2.97 (s, 3H), 2.69-2.65 (m, 1H), 2.55-2.49 (m, 1H), 2.41 (s, 3H), 1.99-1.95 (m, 1H), 1.58-1.50 (m, 2H), 1.39 (d, J=6.6 Hz, 3H), 1.00-0.93 (m, 2H), 0.69-0.64 (m, 2H).

### Example 663

### Synthesis of (1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide (I-663)

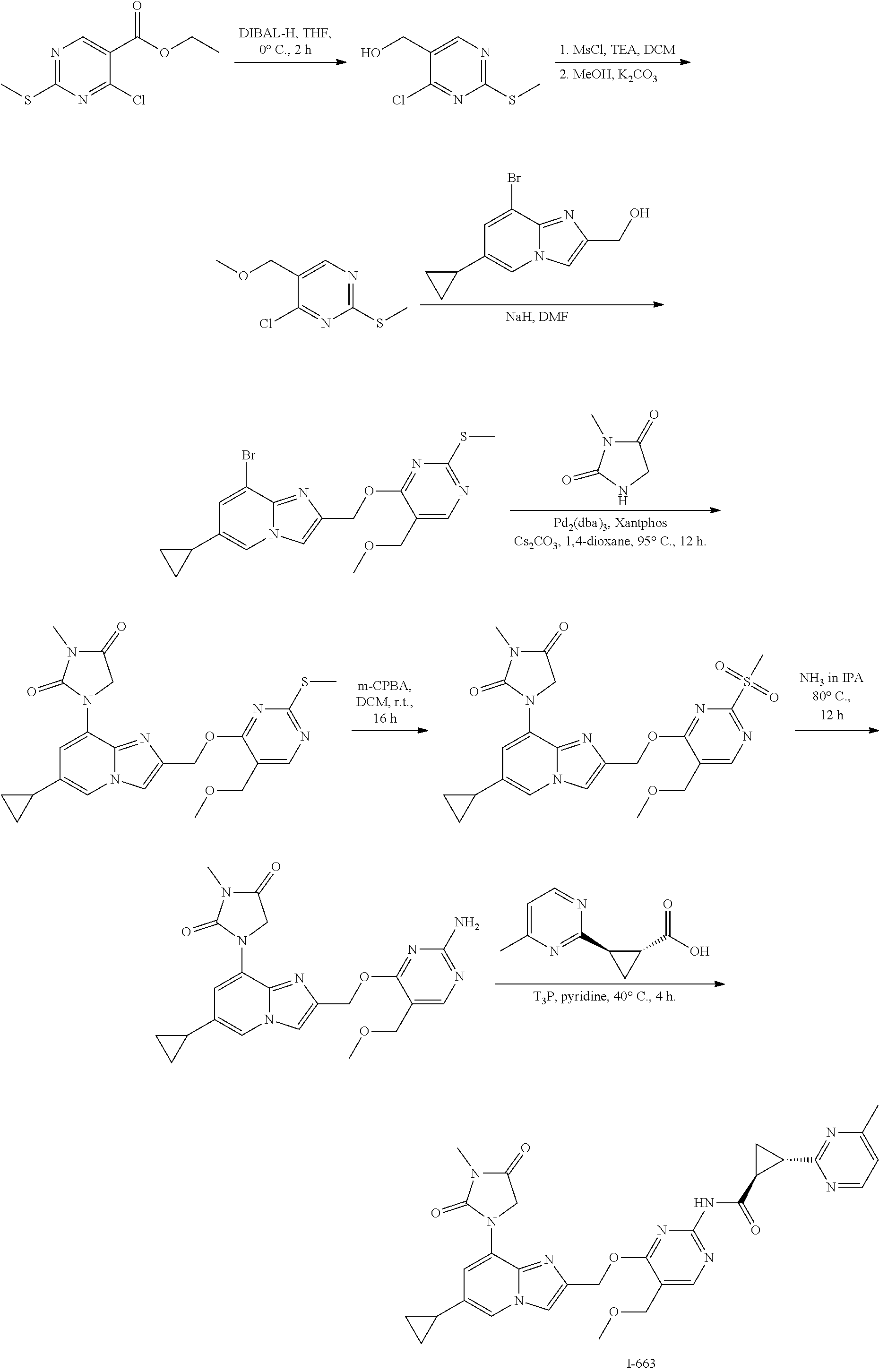

Synthesis of (4-chloro-2-(methylthio)pyrimidin-5-yl)methanol. To a solution of ethyl 4-chloro-2-(methylthio)pyrimidine-5-carboxylate (10 g, 43 mmol) in THF (100 mL) was added DIBAL-H (86 mL, 86 mmol) at 0° C. After stirring at 0° C. for 2 h, the reaction was quenched with Na₂SO₄·10H₂O and filtered. The filtrate was concentrated in vacuo to give (4-chloro-2-(methylthio)pyrimidin-5-yl)methanol (6 g, crude) as a white solid. ESI-MS [M+H]+: 191.1

Synthesis of 4-chloro-5-(methoxymethyl)-2-(methylthio)pyrimidine. To a solution of (4-chloro-2-(methylthio)pyrimidin-5-yl)methanol (6 g, 31.6 mmol) and TEA (9.6 g, 94.8 mmol) in DCM (10 mL) was added MsCl (5.4 g, 47.4 mmol) at 0° C. The mixture was stirred at r.t for 2 h and then concentrated in vacuo. Then, the residue was dissolved in MeOH (50 mL) and K₂CO₃ was added (4.3 g, 31.6 mmol). After stirring at 0° C. for 0.5 h, the reaction was concentrated in vacuo to give the crude, which was purified by column chromatography (eluent: PE/EA=10/1) to give 4-chloro-5-(methoxymethyl)-2-(methylthio)pyrimidine (3.1 g, 48%) as a white solid. ESI-MS [M+H]+: 205.2

Synthesis of 8-bromo-6-cyclopropyl-2-(((5-(methoxymethyl)-2-(methylthio)pyrimidin-4-yl)oxy)methyl)imidazo[1,2-a]pyridine. To a solution of (8-bromo-6-cyclopropylimidazo[1,2-a]pyridin-2-yl)methanol (4.0 g, 15.2 mmol) in THF (50 mL) was added NaH (1.2 g, 30.4 mmol) at 0° C. and the mixture was stirred at 0° C. for 1 h. Then a solution of 4-chloro-5-(methoxymethyl)-2-(methylthio)pyrimidine (3.1 g, 15.2 mmol) in DMF (1 mL) was added and stirred at room temperature for 2 h. The mixture was quenched with water (50 mL) and extracted with EtOAc (3×50 mL). The combined organic layers were washed with brine (50 mL), dried over Na₂SO₄, and concentrated in vacuo to give the crude product, which was purified by column chromatography (eluent: PE/EA=1/1) to give 8-bromo-6-cyclopropyl-2-(((5-(methoxymethyl)-2-(methylthio)pyrimidin-4-yl)oxy)methyl)imidazo[1,2-a]pyridine (5.6 g, 85%) as a white solid. ESI-MS [M+H]+: 435.1

Synthesis of 1-(6-cyclopropyl-2-(((5-(methoxymethyl)-2-(methylthio)pyrimidin-4-yl)oxy)methyl)imidazo[1,2-a]pyridin-8-yl)-3-methylimidazolidine-2,4-dione. To a mixture of 8-bromo-6-cyclopropyl-2-(((5-(methoxymethyl)-2-(methylthio)pyrimidin-4-yl)oxy)methyl)imidazo[1,2-a]pyridine (1.5 g, 3.46 mmol), 3-methylimidazolidine-2,4-dione (1.18 g, 10.38 mmol), and Cs₂CO₃ (3.38 g, 10.38 mmol) in 1,4-dioxane (50 mL) were added Pd₂(dba)₃ (316.6 mg, 0.346 mmol) and Xantphos (400 mg, 0.692 mmol). After stirring at 95° C. for 12 h under N₂, the reaction mixture was filtered through Celite® and the filter cake was washed with DCM/MeOH (10/1, 100 mL). The filtrate was concentrated in vacuo to give the crude, which was purified by column chromatography (eluent: PE/EA=10/1˜1/2) to give the product (1.5 g, 92.6%) as a yellow solid. ESI-MS [M+H]+: 469.2

Synthesis of 1-(6-cyclopropyl-2-(((5-(methoxymethyl)-2-(methylsulfonyl)pyrimidin-4-yl)oxy)methyl)imidazo[1,2-a]pyridin-8-yl)-3-methylimidazolidine-2,4-dione. To a solution of 1-(6-cyclopropyl-2-(((5-(methoxymethyl)-2-(methylthio)pyrimidin-4-yl)oxy)methyl)imidazo[1,2-a]pyridin-8-yl)-3-methylimidazolidine-2,4-dione (1.5 g, 3.2 mmol) in DCM (40 mL) was added m-CPBA (2.2 g, 12.8 mmol) at 0° C. After stirring at room temperature for 16 h, the reaction was quenched with water (50 mL) and extracted with DCM (3×50 mL). The combined organic layers were washed with brine (50 mL), dried over Na₂SO₄, and concentrated in vacuo to give the crude product, which was purified with column chromatography (eluent: DCM/MeOH=10/1) to give the product 1-(6-cyclopropyl-2-(((5-(methoxymethyl)-2-(methylsulfonyl)pyrimidin-4-yl)oxy)methyl)imidazo[1,2-a]pyridin-8-yl)-3-methylimidazolidine-2,4-dione as a red solid. (430 mg, 27%) ESI-MS [M+H]+: 501.2

Synthesis of 1-(2-(((2-amino-5-(methoxymethyl)pyrimidin-4-yl)oxy)methyl)-6-cyclopropylimidazo[1,2-a]pyridin-8-yl)-3-methylimidazolidine-2,4-dione. A mixture of 1-(6-cyclopropyl-2-(((5-(methoxymethyl)-2-(methylsulfonyl)pyrimidin-4-yl)oxy)methyl)imidazo[1,2-a]pyridin-8-yl)-3-methylimidazolidine-2,4-dione (430 mg, 0.86 mmol) in NH₃ (2M in IPA, 30 mL) was stirred at 80° C. for 12 h. The mixture was concentrated in vacuo to give the crude product, which was purified by column chromatography (eluent: PE/EA=1/1) to give the product 1-(2-(((2-amino-5-(methoxymethyl)pyrimidin-4-yl)oxy)methyl)-6-cyclopropylimidazo[1,2-a]pyridin-8-yl)-3-methylimidazolidine-2,4-dione (200 mg, 53%) as a brown solid. ESI-MS [M+H]+: 438.2

Synthesis of (1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide (I-663). To a mixture of 1-(2-(((2-amino-5-(methoxymethyl)pyrimidin-4-yl)oxy)methyl)-6-cyclopropylimidazo[1,2-a]pyridin-8-yl)-3-methylimidazolidine-2,4-dione (200 mg, 0.46 mmol) and (1R,2R)-2-(4-methylpyrimidin-2-yl)cyclopropane-1-carboxylic acid (91 mg, 0.51 mg) in pyridine (1.0 mL) was added T₃P (2.3 g, 3.68 mmol). After stirring at 40° C. for 4 h, the reaction was quenched with saturated aq·NaHCO₃ solution (10 mL) and extracted with EtOAc (20 mL×3). The combined organic layers were washed with brine (30 mL), dried over Na₂SO₄ and concentrated in vacuo to give the crude product, which was purified by preparative TLC (eluent: DCM/MeOH=15/1) to give the product (1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide (100 mg, 36%) as a yellow solid. ESI-MS [M+H]+: 598.2 ¹H NMR (400 MHz, DMSO) δ 10.88 (s, 1H), 8.49 (d, J=5.1 Hz, 1H), 8.32 (s, 1H), 8.27 (d, J=1.2 Hz, 1H), 8.01 (s, 1H), 7.38 (d, J=1.5 Hz, 1H), 7.16 (d, J=5.1 Hz, 1H), 5.48 (s, 2H), 4.88 (s, 2H), 4.30 (s, 2H), 3.26 (s, 3H), 2.97 (s, 3H), 2.63-2.57 (m, 1H), 2.55-2.51 (m, 1H), 2.39 (s, 3H), 2.01-1.91 (m, 1H), 1.61-1.54 (m, 2H), 0.98-0.90 (m, 2H), 0.72-0.63 (m, 2H).

### Example 664

### Synthesis of (1S,2S)—N-(4-((6-cyclopropyl-8-(3-methyl-2,4-dioxoimidazolidin-1-yl)imidazo[1,2-a]pyridin-2-yl)methoxy)-5-(hydroxymethyl)pyrimidin-2-yl)-2-(4-methylpyrimidin-2-yl)cyclopropane-1-carboxamide (I-664)

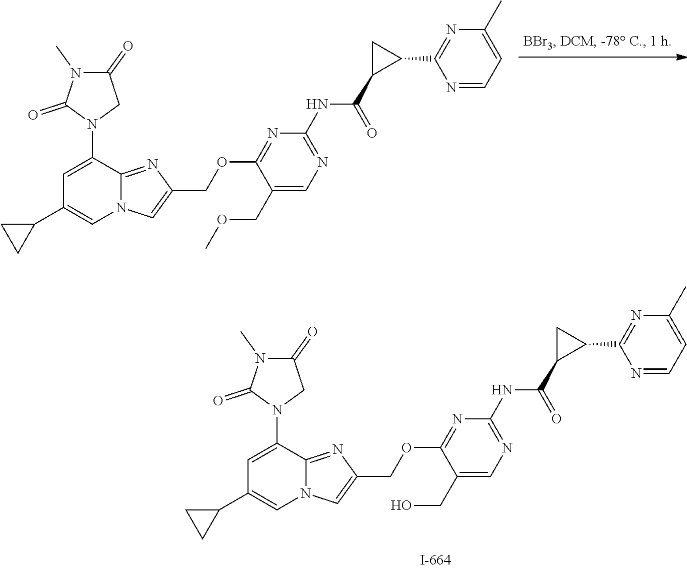 [SMILES: COCc1cnc(NC(=O)[C@H]2C[C@@H]2c2nccc(C)n2)nc1OCc1cn2cc(C3CC3)cc(N3CC(=O)N(C)C3=O)c2n1 ; Cc1ccnc([C@H]2C[C@@H]2C(=O)Nc2ncc(CO)c(OCc3cn4cc(C5CC5)cc(N5CC(=O)N(C)C5=O)c4n3)n2)n1]

To a solution of (1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide (50 mg, 0.08 mmol) in DCM (5 mL) was added BBr₃ (100 mg, 0.40 mmol) at −78° C. After stirring at −78° C. for 1 h, the reaction was quenched with MeOH (1 mL). Then the mixture was concentrated in vacuo to give the crude, which was purified by preparative TLC (eluent: DCM/MeOH=10/1) to give the (1S,2S)—N-(4-((6-cyclopropyl-8-(3-methyl-2,4-dioxoimidazolidin-1-yl)imidazo[1,2-a]pyridin-2-yl)methoxy)-5-(hydroxymethyl)pyrimidin-2-yl)-2-(4-methylpyrimidin-2-yl)cyclopropane-1-carboxamide as a white solid. (7 mg, 15%). ESI-MS [M+H]+: 584.3 ¹H NMR (400 MHz, DMSO) δ 10.82 (s, 1H), 8.49 (d, J=5.1 Hz, 1H), 8.31 (s, 1H), 8.27 (s, 1H), 8.02 (s, 1H), 7.38 (d, J=1.4 Hz, 1H), 7.16 (d, J=5.1 Hz, 1H), 5.47 (s, 2H), 5.15 (t, J=5.5 Hz, 1H), 4.88 (s, 2H), 4.39 (d, J=5.4 Hz, 2H), 2.97 (s, 3H), 2.61-2.57 (m, 1H), 2.55-2.52 (m, 1H), 2.39 (s, 3H), 2.00-1.94 (m, 1H), 1.60-1.55 (m, 2H), 1.01-0.88 (m, 2H), 0.69-0.65 (m, 2H). ESI-MS [M+H]+: 584.3

### Example 665

### Synthesis of (1S,2S)—N-(5-(((R)-1-(6-cyclopropyl-8-(3-methyl-2,4-dioxoimidazolidin-1-yl) imidazo[1,2-a]pyridin-2-yl)ethyl)amino)-1,2,4-triazin-3-yl)-2-(4-methylpyrimidin-2-yl)cyclopropane-1-carboxamide (I-665)

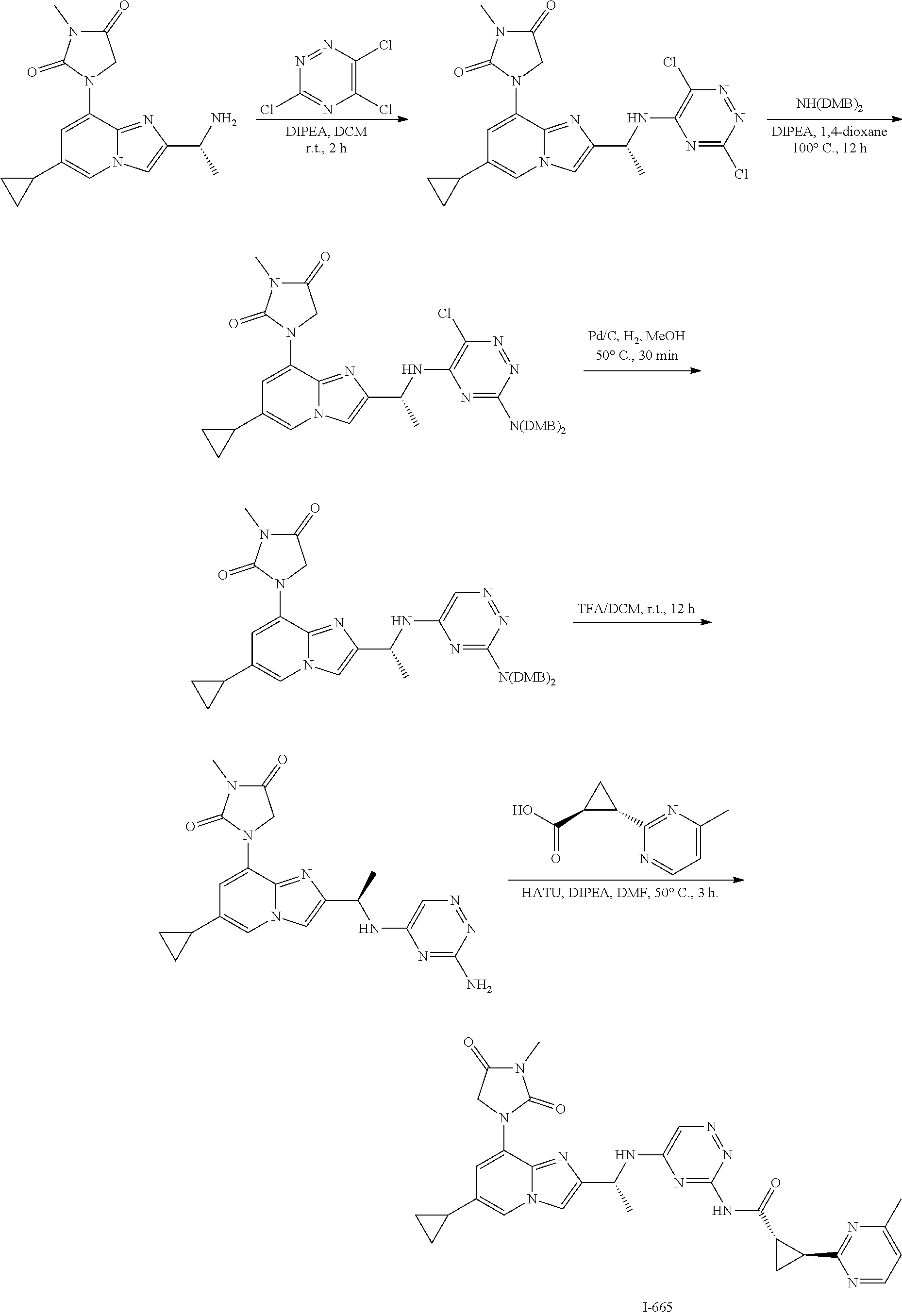

Synthesis of (R)-1-(6-cyclopropyl-2-(1-((3,6-dichloro-1,2,4-triazin-5-yl)amino)ethyl)imidazo[1,2-a]pyridin-8-yl)-3-methylimidazolidine-2,4-dione. A mixture of (R)-1-(2-(1-aminoethyl)-6-cyclopropylimidazo[1,2-a]pyridin-8-yl)-3-methylimidazolidine-2,4-dione (600 mg, 1.91 mmol), 3,5,6-trichloro-1,2,4-triazine (527 mg, 2.87 mmol) and DIPEA (740 mg, 5.73 mmol) in DCM (12 mL) was stirred in a sealed tube. After degassing with N₂ for 1 min, the mixture was stirred at room temperature for 2 h. The mixture was concentrated in vacuo to give the crude product, which was purified by preparative TLC (eluent: DCM/MeOH=20/1) to give (R)-1-(6-cyclopropyl-2-(1-((3,6-dichloro-1,2,4-triazin-5-yl)amino)ethyl)imidazo[1,2-a]pyridin-8-yl)-3-methylimidazolidine-2,4-dione (600 mg, 68%) as a yellow solid. ESI-MS [M+H]+: 461.2.

Synthesis of (R)-1-(2-(1-((3-(bis(3,4-dimethylbenzyl)amino)-6-chloro-1,2,4-triazin-5-yl)amino)ethyl)-6-cyclopropylimidazo[1,2-a]pyridin-8-yl)-3-methylimidazolidine-2,4-dione. A mixture of (R)-1-(6-cyclopropyl-2-(1-((3,6-dichloro-1,2,4-triazin-5-yl)amino)ethyl)imidazo[1,2-a]pyridin-8-yl)-3-methylimidazolidine-2,4-dione (600 mg, 1.3 mmol), NH(DMB)₂ (658 mg, 2.6 mmol) and DIPEA (500 mg, 3.9 mmol) in DMF (10 mL) was stirred at 100° C. for 12 h under N₂. Water (20 mL) was added and the mixture was extracted with DCM (3×20 mL). The combined organic layers were washed with brine, dried over Na₂SO₄, filtered, and concentrated in vacuo to give the crude, which was purified by preparative TLC (eluent: DCM/MeOH=20/1) to give (R)-1-(2-(1-((3-(bis(3,4-dimethylbenzyl)amino)-6-chloro-1,2,4-triazin-5-yl)amino)ethyl)-6-cyclopropylimidazo[1,2-a]pyridin-8-yl)-3-methylimidazolidine-2,4-dione (500 mg, 58%) as a yellow solid.

ESI-MS [M+H]+: 678.2

Synthesis of (R)-1-(2-(1-((3-(bis(3,4-dimethylbenzyl)amino)-1,2,4-triazin-5-yl)amino)ethyl)-6-cyclopropylimidazo[1,2-a]pyridin-8-yl)-3-methylimidazolidine-2,4-dione. A mixture of (R)-1-(2-(1-((3-(bis(3,4-dimethylbenzyl)amino)-6-chloro-1,2,4-triazin-5-yl)amino)ethyl)-6-cyclopropylimidazo[1,2-a]pyridin-8-yl)-3-methylimidazolidine-2,4-dione (500 mg, 0.73 mmol) and Pd/C (300 mg) in MeOH (20 mL) was stirred at 50° C. for 0.5 h under H₂. The reaction mixture was filtered through Celite® and the filter cake was washed with DCM/MeOH (10/1, 20 mL). The filtrate was concentrated in vacuo to give the crude, which was purified by preparative HPLC to give (R)-1-(2-(1-((3-(bis(3,4-dimethylbenzyl)amino)-1,2,4-triazin-5-yl)amino)ethyl)-6-cyclopropylimidazo[1,2-a]pyridin-8-yl)-3-methylimidazolidine-2,4-dione (380 mg, 80%) as a white solid. ESI-MS [M+H]⁺: 644.1.

Synthesis of (R)-1-(2-(1-((3-amino-1,2,4-triazin-5-yl)amino)ethyl)-6-cyclopropylimidazo[1,2-a]pyridin-8-yl)-3-methylimidazolidine-2,4-dione. A mixture of (R)-1-(2-(1-((3-(bis(3,4-dimethylbenzyl)amino)-1,2,4-triazin-5-yl)amino)ethyl)-6-cyclopropylimidazo[1,2-a]pyridin-8-yl)-3-methylimidazolidine-2,4-dione (380 mg, 0.59 mmol) in TFA (4 mL) was stirred at room temperature for 12 h. The reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC (eluent: DCM/MeOH=10/1) to give (R)-1-(2-(1-((3-amino-1,2,4-triazin-5-yl)amino)ethyl)-6-cyclopropylimidazo[1,2-a]pyridin-8-yl)-3-methylimidazolidine-2,4-dione (120 mg, 50%) as a white solid. ESI-MS [M+H]+: 408.2.

Synthesis of (1S,2S)—N-(5-(((R)-1-(6-cyclopropyl-8-(3-methyl-2,4-dioxoimidazolidin-1-yl)imidazo[1,2-a]pyridin-2-yl)ethyl)amino)-1,2,4-triazin-3-yl)-2-(4-methylpyrimidin-2-yl)cyclopropane-1-carboxamide. A mixture of (R)-1-(2-(1-((3-amino-1,2,4-triazin-5-yl)amino)ethyl)-6-cyclopropylimidazo[1,2-a]pyridin-8-yl)-3-methylimidazolidine-2,4-dione (120 mg, 0.29 mmol), (1S,2S)-2-(4-methylpyrimidin-2-yl)cyclopropane-1-carboxylic acid (34 mg, 0.29 mmol), HATU (220 mg, 0.58 mmol), and DIPEA (112 mg, 0.87 mmol) in DMF (4 mL) was stirred at 50° C. for 3 h under N₂. The reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC(eluent: DCM/MeOH=20/1) to give (1S,2S)—N-(5-(((R)-1-(6-cyclopropyl-8-(3-methyl-2,4-dioxoimidazolidin-1-yl)imidazo[1,2-a]pyridin-2-yl)ethyl)amino)-1,2,4-triazin-3-yl)-2-(4-methylpyrimidin-2-yl)cyclopropane-1-carboxamide (95 mg, 58%) as a yellow solid. ESI-MS [M+H]+: 568.2, ¹H NMR (400 MHz, DMSO) δ 10.60 (s, 1H), 8.54 (d, J=7.9 Hz, 1H), 8.48 (d, J=5.1 Hz, 1H), 8.32 (s, 1H), 8.21 (s, 1H), 7.75 (s, 1H), 7.34 (d, J=1.4 Hz, 1H), 7.15 (d, J=5.1 Hz, 1H), 5.26-5.23 (m, 1H), 4.91 (s, 2H), 2.97 (s, 3H), 2.57-2.52 (m, 2H), 2.37 (s, 3H), 1.97-1.93 (m, 1H), 1.55-1.52 (m, 5H), 0.96-0.92 (m, 2H), 0.66-0.62 (m, 2H).

### Example 666

### Synthesis of N-(5-((6-cyclopropyl-8-(3-methyl-2,4-dioxoimidazolidin-1-yl)imidazo[1,2-a]pyridin-2-yl)methoxy)-6-(trifluoromethyl)pyridazin-3-yl)-2-(4-methylpyrimidin-2-yl)cyclopropane-1-carboxamide (I-666)

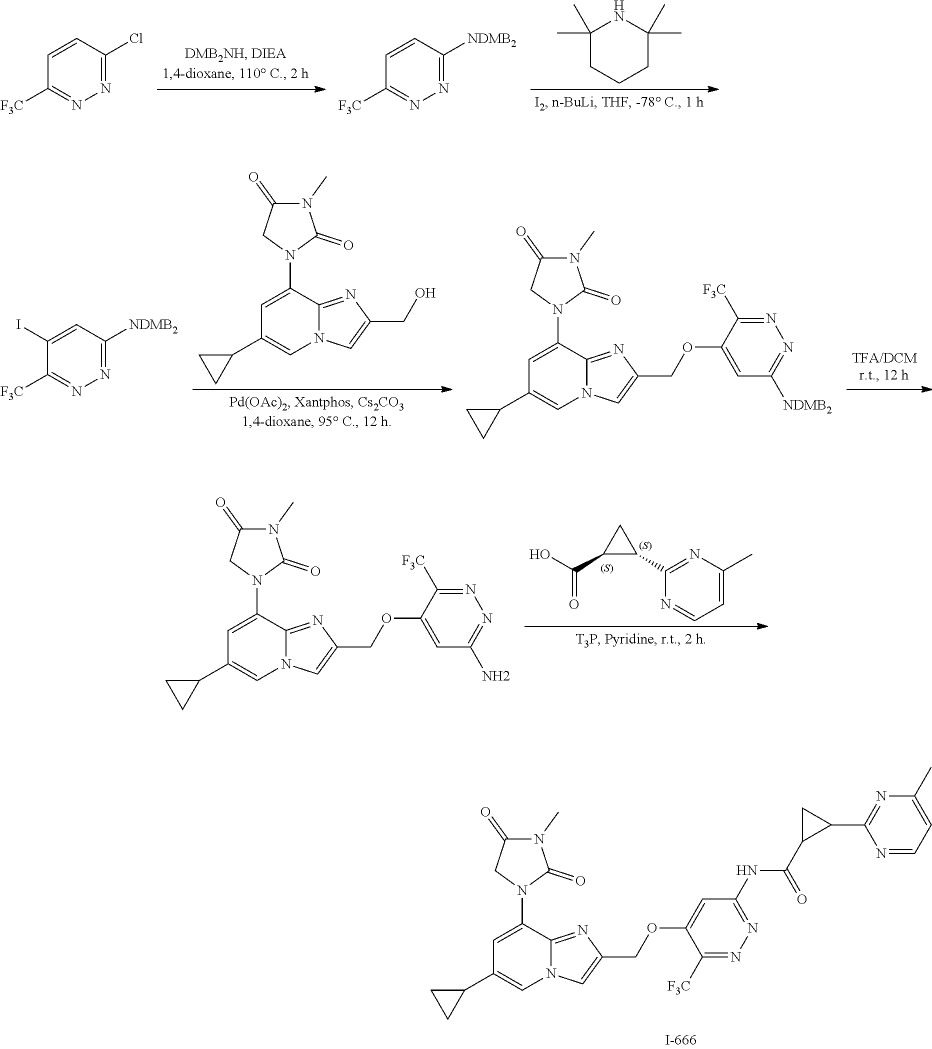

Synthesis of N,N-bis(3,4-dimethylbenzyl)-6-(trifluoromethyl)pyridazin-3-amine. To a solution of 3-chloro-6-(trifluoromethyl)pyridazine (5 g, 27.4 mmol) in 1,4-dioxane (50 mL) was added DMB₂NH (9.5 g, 30.1 mmol) and DIEA (10.6 g, 82.2 mmol) at room temperature. After stirring at 110° C. for 2 h, water (200 mL) was added and the mixture was extracted with DCM (3×50 mL). The combined organic layers were washed with brine, dried over Na₂SO₄, filtered and concentrated in vacuo to give the crude, which was purified by column chromatography (eluent: PE/EtOAc=20/1˜5/1) to give N,N-bis(3,4-dimethylbenzyl)-6-(trifluoromethyl)pyridazin-3-amine (7 g, 55%) as a yellow solid. ESI-MS [M+H]+: 464.2

Synthesis of N,N-bis(3,4-dimethoxybenzyl)-5-iodo-6-(trifluoromethyl)pyridazin-3-amine. To a solution of 2,2,6,6-tetramethylpiperidine (423 mg, 0.9 mmol) in THF (10 mL) was added n-BuLi (0.5 mL, 1.2 mmol) at −78° C. under N₂. The mixture was stirred at −78° C. for 1 h and then a solution of N,N-bis(3,4-dimethylbenzyl)-6-(trifluoromethyl)pyridazin-3-amine (463 mg, 1.0 mmol) in THF (1 mL) was added slowly. After 1 h, a solution of I₂ (379.5 mg, 1.5 mmol) in THF (1 mL) was added and the reaction mixture was stirred at −78° C. for 1 h. The reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by preparative TLC (eluent: PE/EtOAc=5/1) to give N,N-bis(3,4-dimethoxybenzyl)-5-iodo-6-(trifluoromethyl)pyridazin-3-amine (463 mg, 87%) as a yellow solid. ESI-MS [M+H]+: 590.2

Synthesis of 1-(2-(((6-(bis(3,4-dimethylbenzyl)amino)-3-(trifluoromethyl)pyridazin-4-yl)oxy)methyl)-6-cyclopropylimidazo[1,2-a]pyridin-8-yl)-3-methylimidazolidine-2,4-dione. To a mixture of N,N-bis(3,4-dimethoxybenzyl)-5-iodo-6-(trifluoromethyl)pyridazin-3-amine (138 mg, 0.46 mmol), 1-(6-cyclopropyl-2-(hydroxymethyl)imidazo[1,2-a]pyridin-8-yl)-3-methylimidazolidine-2,4-dione (270 mg, 0.46 mmol) and Cs₂CO₃ (449.6 mg, 1.4 mmol) in 1,4-dioxane (5 mL) were added Pd(OAc)₂ (24.1 mg, 0.09 mmol) and Xantphos (79.8 mg, 0.14 mmol). After stirring at 95° C. for 12 h under N₂, the reaction mixture was filtered through Celite® and the filter cake was washed with DCM/MeOH (10/1, 10 mL). The filtrate was concentrated in vacuo to give the crude, which was purified by preparative TLC (eluent: DCM/MeOH=20/1) to give 1-(2-(((6-(bis(3,4-dimethylbenzyl)amino)-3-(trifluoromethyl)pyridazin-4-yl)oxy)methyl)-6-cyclopropylimidazo[1,2-a]pyridin-8-yl)-3-methylimidazolidine-2,4-dione (160 mg, 46%) as a yellow solid. ESI-MS [M+H]+: 762.2.

Synthesis of 1-(2-(((6-amino-3-(trifluoromethyl)pyridazin-4-yl)oxy)methyl)-6-cyclopropylimidazo[1,2-a]pyridin-8-yl)-3-methylimidazolidine-2,4-dione. To a mixture of 1-(2-(((6-(bis(3,4-dimethylbenzyl)amino)-3-(trifluoromethyl)pyridazin-4-yl)oxy)methyl)-6-cyclopropylimidazo[1,2-a]pyridin-8-yl)-3-methylimidazolidine-2,4-dione (300 mg, 0.58 mmol) in DCM (4 mL) was added TFA (4 ml) at room temperature. The resulting reaction was stirred at room temperature for 12 h. The reaction was quenched with water (10 mL) and extracted with EtOAc (3×10 mL). The combined organic layers were washed with brine (20 mL), dried over anhydrous Na₂SO₄, and concentrated in vacuo to give the crude product, which was purified by preparative TLC (eluent: DCM/MeOH=10/1) to give 1-(2-(((6-amino-3-(trifluoromethyl)pyridazin-4-yl)oxy)methyl)-6-cyclopropylimidazo[1,2-a]pyridin-8-yl)-3-methylimidazolidine-2,4-dione (140 mg, 52%) as a white solid. ESI-MS [M+H]+: 462.2.

Synthesis of N-(5-((6-cyclopropyl-8-(3-methyl-2,4-dioxoimidazolidin-1-yl)imidazo[1,2-a]pyridin-2-yl)methoxy)-6-(trifluoromethyl)pyridazin-3-yl)-2-(4-methylpyrimidin-2-yl)cyclopropane-1-carboxamide (I-666). A mixture of 1-(2-(((6-amino-3-(trifluoromethyl)pyridazin-4-yl)oxy)methyl)-6-cyclopropylimidazo[1,2-a]pyridin-8-yl)-3-methylimidazolidine-2,4-dione (50 mg, 0.1 mmol), (1S,2S)-2-(4-methylpyrimidin-2-yl)cyclopropane-1-carboxylic acid (19 mg, 0.1 mmol) and T₃P (343.0 mg, 1.1 mmol) in pyridine (1 mL) was stirred at room temperature for 2 h. Water (20 mL) was added and the mixture was extracted with EtOAc (3×20 mL). The combined organic layers were washed with brine, dried over Na₂SO₄, filtered and concentrated in vacuo to give the crude, which was purified by preparative TLC (eluent: DCM/MeOH=201) to give N-(5-((6-cyclopropyl-8-(3-methyl-2,4-dioxoimidazolidin-1-yl)imidazo[1,2-a]pyridin-2-yl)methoxy)-6-(trifluoromethyl)pyridazin-3-yl)-2-(4-methylpyrimidin-2-yl)cyclopropane-1-carboxamide (38 mg, 61%) as a white solid. ESI-MS [M+H]+: 622.1. ¹H NMR (400 MHz, DMSO) δ 11.83 (s, 1H), 8.62 (d, J=28.2 Hz, 1H), 8.55 (d, J=5.0 Hz, 1H), 8.31 (s, 1H), 8.01 (s, 1H), 7.44 (s, 1H), 7.23 (d, J=5.0 Hz, 1H), 5.53 (s, 2H), 4.96-4.94 (m, 2H), 2.99 (s, 3H), 2.67-2.56 (m, 2H), 2.43 (s, 3H), 1.99-1.96 (m, 1H), 1.67-1.46 (m, 2H), 0.98-0.94 (m, 2H), 0.69-0.65 (m, 2H).

### Example 667

### Synthesis of (1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (I-667)

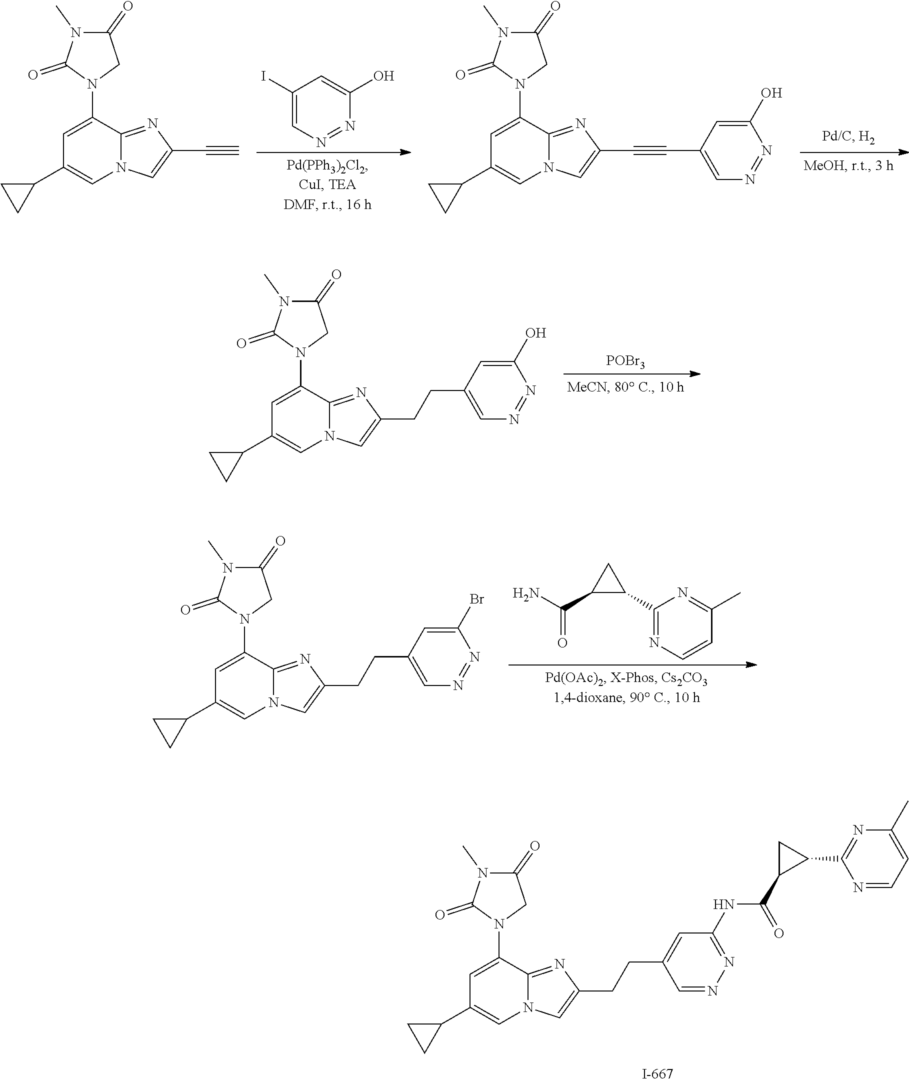

Synthesis of 1-(6-cyclopropyl-2-((6-hydroxypyridazin-4-yl)ethynyl)imidazo[1,2-a]pyridin-8-yl)-3-methylimidazolidine-2,4-dione. To a reaction mixture of 1-(6-cyclopropyl-2-ethynylimidazo[1,2-a]pyridin-8-yl)-3-methylimidazolidine-2,4-dione (0.37 g, 0.50 mmol), Pd (PPh₃)₂Cl₂ (0.10 g, 0.13 mmol), CuI (0.12 g, 0.63 mmol), and TEA (0.64 g, 5.0 mmol) in DMF (10 mL) was added 5-iodopyridazin-3-ol (0.56 g, 2.5 mmol). After stirring at room temperature for 16 h, the reaction was concentrated in vacuo to give the crude, which was purified by column chromatography on silica gel (eluent: MeOH/DCM=1/20) to give the 1-(6-cyclopropyl-2-((6-hydroxypyridazin-4-yl)ethynyl)imidazo[1,2-a]pyridin-8-yl)-3-methylimidazolidine-2,4-dione (0.20 g, 41%) as yellow solid. ESI-MS [M+H]⁺: 389.1.

Synthesis of 1-(6-cyclopropyl-2-(2-(6-hydroxypyridazin-4-yl)ethyl)imidazo[1,2-a]pyridin-8-yl)-3-methylimidazolidine-2,4-dione. A reaction mixture of 1-(6-cyclopropyl-2-((6-hydroxypyridazin-4-yl)ethynyl)imidazo[1,2-a]pyridin-8-yl)-3-methylimidazolidine-2,4-dione (0.20 g, 0.52 mmol) and 10% Pd on carbon (0.20 g) in MeOH (20 mL) was stirred at room temperature for 3 h under H₂ atmosphere. The mixture was then filtered and concentrated in vacuo to give the 1-(6-cyclopropyl-2-(2-(6-hydroxypyridazin-4-yl)ethyl)imidazo[1,2-a]pyridin-8-yl)-3-methylimidazolidine-2,4-dione (0.18 g, 89%) as yellow solid, which was used in next step without purification. ESI-MS [M+H]⁺: 393.2.

Synthesis of 1-(2-(2-(6-bromopyridazin-4-yl)ethyl)-6-cyclopropylimidazo[1,2-a]pyridin-8-yl)-3-methylimidazolidine-2,4-dione. A mixture of 1-(6-cyclopropyl-2-(2-(6-hydroxypyridazin-4-yl)ethyl)imidazo[1,2-a]pyridin-8-yl)-3-methylimidazolidine-2,4-dione (0.16 g, 0.40 mmol) in MeCN (15 mL) was added POBr₃ (1.1 g, 4.0 mmol). Then the reaction was stirred at 80° C. for 10 h under N₂. After cooling to room temperature, the reaction was quenched in ice/water and neutralized with solid NaHCO₃. The product was extracted with EtOAc and the organic phase was washed with brine, dried over Na₂SO₄ and concentrated in vacuo to give the crude, which was purified by column chromatography on silica gel (eluent: MeOH/DCM=1/20) to give the 1-(2-(2-(6-bromopyridazin-4-yl)ethyl)-6-cyclopropylimidazo[1,2-a]pyridin-8-yl)-3-methylimidazolidine-2,4-dione (95 mg, 52%) as yellow solid. ESI-MS [M+H]⁺: 455.1.

Synthesis of (1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (I-667). To a reaction mixture of 1-(2-(2-(6-bromopyridazin-4-yl)ethyl)-6-cyclopropylimidazo[1,2-a]pyridin-8-yl)-3-methylimidazolidine-2,4-dione (0.14 g, 0.50 mmol), (1S,2S)-2-(4-methylpyrimidin-2-yl)cyclopropane-1-carboxamide (36 mg, 0.20 mmol), Cs₂CO₃ (0.20 mg, 0.60 mmol), and X-Phos (19 mg, 0.040 mmol) in 1,4-dioxane (10 mL) was added Pd(OAc)₂ (4.0 mg, 0.020 mmol). The reaction was stirred at 90° C. for 10 h under N₂ and cooled to room temperature. The reaction was concentrated in vacuo to give the crude, which was purified by column chromatography on silica gel (eluent: MeOH/DCM=1/15) to give the (1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (19 mg, 17%) as white solid. ESI-MS [M+H]⁺: 552.2. ¹H NMR (400 MHz, DMSO) δ 11.35 (s, 1H), 8.85 (d, J=1.9 Hz, 1H), 8.53 (d, J=5.1 Hz, 1H), 8.19 (d, J=1.2 Hz, 2H), 7.62 (s, 1H), 7.32 (d, J=1.5 Hz, 1H), 7.22 (d, J=5.1 Hz, 1H), 4.92-4.87 (m, 2H), 3.09-3.02 (m, 4H), 2.98 (s, 3H), 2.69-2.67 (m, 1H), 2.56-2.54 (m, 1H), 2.42 (s, 3H), 1.97-1.91 (m, 1H), 1.58-1.49 (m, 2H), 0.97-0.90 (m, 2H), 0.68-0.62 (m, 2H).

### Example 668

### Synthesis of (1S,2S)—N-(5-(((6-cyclopropyl-8-(3-methyl-2,4-dioxoimidazolidin-1-yl)imidazo[1,2-a]pyridin-2-yl)methyl)(methyl)amino)-1,2,4-triazin-3-yl)-2-(4-methylpyrimidin-2-yl)cyclopropane-1-carboxamide (I-668)

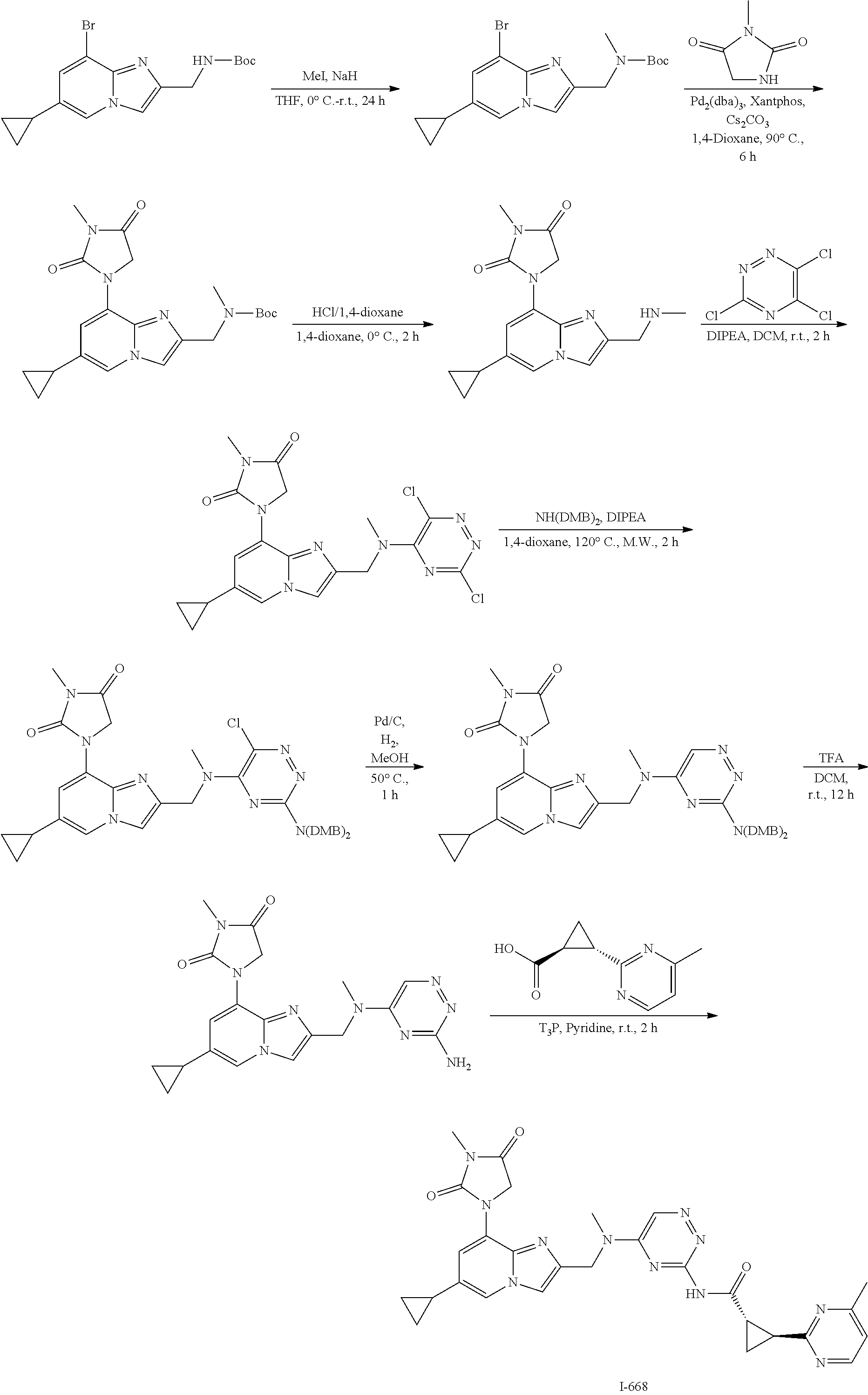

Synthesis of tert-butyl ((8-bromo-6-cyclopropylimidazo[1,2-a]pyridin-2-yl)methyl)(methyl)carbamate. To a mixture of tert-butyl ((8-bromo-6-cyclopropylimidazo[1,2-a]pyridin-2-yl)methyl)carbamate (2.0 g, 5.5 mmol) in THF (35 mL) was added NaH (0.40 g, 9.8 mmol, 60% purity in mineral oil) at 0° C. for 30 min. Then a solution of Mel (1.712 g, 12.054 mmol) in THF (5 mL) was added and stirred at room temperature for 24 h. The mixture was quenched with saturated aq·NH₄Cl (50 mL) and extracted with EtOAc (3×100 mL). The combined organic extract was washed with brine (200 mL), dried over Na₂SO₄, concentrated in vacuo and purified by column chromatography on silica gel (eluent: PE/EtOAc=2/1) to give tert-butyl ((8-bromo-6-cyclopropylimidazo[1,2-a]pyridin-2-yl)methyl)(methyl)carbamate (1.3 g, 60%) as white solid. ESI-MS [M+H]+: 380.1.

Synthesis of tert-butyl ((6-cyclopropyl-8-(3-methyl-2,4-dioxoimidazolidin-1-yl)imidazo[1,2-a]pyridin-2-yl)methyl)(methyl)carbamate. A mixture of tert-butyl ((8-bromo-6-cyclopropylimidazo[1,2-a]pyridin-2-yl)methyl)(methyl)carbamate (1.2 g, 3.1 mmol), 3-methylimidazolidine-2,4-dione (0.70 g, 6.2 mmol), Pd₂(dba)₃ (0.57 g, 0.62 mmol), Xantphos (0.72 mg, 1.2 mmol), and Cs₂CO₃ (3.0 mg, 9.3 mmol) in 1,4-dioxane (40 mL) was stirred at 90° C. under N₂ for 6 h. Water (50 mL) was added, and the mixture extracted with EtOAc (3×80 mL). The combined organic layers were washed with brine (100 mL), dried over anhydrous Na₂SO₄, filtered and concentrated in vacuo. The residue was purified by column chromatography on silica gel (eluent: PE/EtOAc=2/1) to give tert-butyl ((6-cyclopropyl-8-(3-methyl-2,4-dioxoimidazolidin-1-yl)imidazo[1,2-a]pyridin-2-yl)methyl)(methyl)carbamate (0.78 g, 61%) as yellow solid. ESI-MS [M+H]+: 414.2.

Synthesis of 1-(6-cyclopropyl-2-((methylamino)methyl)imidazo[1,2-a]pyridin-8-yl)-3-methylimidazolidine-2,4-dione. To a mixture of tert-butyl ((6-cyclopropyl-8-(3-methyl-2,4-dioxoimidazolidin-1-yl)imidazo[1,2-a]pyridin-2-yl)methyl)(methyl)carbamate (0.78 g, 1.9 mmol) in 1,4-dioxane (3 mL) was added HCl/dioxane (4M in dioxane, 15 mL). The mixture was stirred at 0° C. for 2 h. The mixture was concentrated in vacuo and purified by preparative TLC (eluent: DCM/MeOH=10/1) to give 1-(6-cyclopropyl-2-((methylamino)methyl)imidazo[1,2-a]pyridin-8-yl)-3-methylimidazolidine-2,4-dione (0.53 g, 90%) as yellow solid. ESI-MS [M+H]+: 314.2.

Synthesis of 1-(6-cyclopropyl-2-(((3,6-dichloro-1,2,4-triazin-5-yl)(methyl)amino)methyl)imidazo[1,2-a]pyridin-8-yl)-3-methylimidazolidine-2,4-dione. A mixture of 1-(6-cyclopropyl-2-((methylamino)methyl)imidazo[1,2-a]pyridin-8-yl)-3-methylimidazolidine-2,4-dione (0.26 g, 0.84 mmol), 3,5,6-trichloro-1,2,4-triazine (0.23 g, 1.3 mmol), and DIPEA (0.54 g, 4.2 mmol) in DCM (15 mL) was stirred at room temperature for 2 h. The mixture was concentrated in vacuo and purified by preparative TLC (eluent: PE/EtOAc=1/1) to give 1-(6-cyclopropyl-2-(((3,6-dichloro-1,2,4-triazin-5-yl)(methyl)amino)methyl)imidazo[1,2-a]pyridin-8-yl)-3-methylimidazolidine-2,4-dione (0.33 g, 85%) as yellow solid. ESI-MS [M+H]+: 293.1.

Synthesis of 1-(2-(((3-(bis(2,4-dimethoxybenzyl)amino)-6-chloro-1,2,4-triazin-5-yl)(methyl)amino)methyl)-6-cyclopropylimidazo[1,2-a]pyridin-8-yl)-3-methylimidazolidine-2,4-dione. A mixture of 1-(6-cyclopropyl-2-(((3,6-dichloro-1,2,4-triazin-5-yl)(methyl)amino)methyl)imidazo[1,2-a]pyridin-8-yl)-3-methylimidazolidine-2,4-dione (0.33 g, 0.72 mmol), NH(DMB)₂ (0.68 g, 2.2 mmol), and DIPEA (0.28 g, 2.2 mmol) in 1,4-dioxane (8.0 mL) was stirred at 120° C. in a microwave reactor for 3 h. The mixture was concentrated in vacuo and purified by preparative TLC (eluent: PE/EtOAc=1/1) to give 1-(2-(((3-(bis(2,4-dimethoxybenzyl)amino)-6-chloro-1,2,4-triazin-5-yl)(methyl)amino)methyl)-6-cyclopropylimidazo[1,2-a]pyridin-8-yl)-3-methylimidazolidine-2,4-dione (0.48 g, 90%) as light-yellow solid. ESI-MS [M+H]+: 742.2.

Synthesis of 1-(2-(((3-(bis(2,4-dimethoxybenzyl)amino)-1,2,4-triazin-5-yl)(methyl)amino)methyl)-6-cyclopropylimidazo[1,2-a]pyridin-8-yl)-3-methylimidazolidine-2,4-dione. A mixture of 1-(2-(((3-(bis(2,4-dimethoxybenzyl)amino)-6-chloro-1,2,4-triazin-5-yl)(methyl)amino)methyl)-6-cyclopropylimidazo[1,2-a]pyridin-8-yl)-3-methylimidazolidine-2,4-dione (0.24 g, 0.32 mmol) and 10% Pd on carbon (0.21 g, 1.9 mmol) in MeOH (20 mL) was stirred at 50° C. for 1 h under H₂. The mixture was filtered and concentrated in vacuo to give the crude product, which was purified by preparative TLC (eluent: DCM/MeOH=10/1) to give 1-(2-(((3-(bis(2,4-dimethoxybenzyl)amino)-1,2,4-triazin-5-yl)(methyl)amino)methyl)-6-cyclopropylimidazo[1,2-a]pyridin-8-yl)-3-methylimidazolidine-2,4-dione (0.15 mg, 66%) as a yellow solid. ESI-MS [M+H]+: 708.3.

Synthesis of 1-(2-(((3-amino-1,2,4-triazin-5-yl)(methyl)amino)methyl)-6-cyclopropylimidazo[1,2-a]pyridin-8-yl)-3-methylimidazolidine-2,4-dione. A mixture of 1-(2-(((3-(bis(2,4-dimethoxybenzyl)amino)-1,2,4-triazin-5-yl)(methyl)amino)methyl)-6-cyclopropylimidazo[1,2-a]pyridin-8-yl)-3-methylimidazolidine-2,4-dione (0.15 g, 0.21 mmol) and TFA (0.73 g, 6.4 mmol) in DCM (10 mL) was stirred at room temperature for 12 h. The mixture was concentrated in vacuo and purified by preparative TLC (eluent: DCM/MeOH=20/1) to give 1-(2-(((3-amino-1,2,4-triazin-5-yl)(methyl)amino)methyl)-6-cyclopropylimidazo[1,2-a]pyridin-8-yl)-3-methylimidazolidine-2,4-dione (75 mg, 87%) as white solid. ESI-MS [M+H]+: 408.1.

Synthesis of (1S,2S)—N-(5-(((6-cyclopropyl-8-(3-methyl-2,4-dioxoimidazolidin-1-yl)imidazo[1,2-a]pyridin-2-yl)methyl)(methyl)amino)-1,2,4-triazin-3-yl)-2-(4-methylpyrimidin-2-yl)cyclopropane-1-carboxamide (I-668). A mixture of 1-(2-(((3-amino-1,2,4-triazin-5-yl)(methyl)amino)methyl)-6-cyclopropylimidazo[1,2-a]pyridin-8-yl)-3-methylimidazolidine-2,4-dione (75 mg, 0.18 mmol), (1S,2S)-2-(4-methylpyrimidin-2-yl)cyclopropane-1-carboxylic acid (49 mg, 0.28 mmol) and T₃P (0.94 g, 1.5 mmol) in pyridine (0.38 mL) was stirred at room temperature for 2 h. The mixture was quenched with saturated aq·NaHCO₃ (5.0 mL) and extracted with EtOAc (3×20 mL). The combined organic extract was washed with brine (20 mL), dried over Na₂SO₄, and concentrated in vacuo and purified by preparative TLC (eluent: DCM/MeOH=10/1) to give (1S,2S)—N-(5-(((6-cyclopropyl-8-(3-methyl-2,4-dioxoimidazolidin-1-yl)imidazo[1,2-a]pyridin-2-yl)methyl)(methyl)amino)-1,2,4-triazin-3-yl)-2-(4-methylpyrimidin-2-yl)cyclopropane-1-carboxamide (16 mg, 15%) as yellow solid. ESI-MS [M+H]+: 568.3. ¹H NMR (400 MHz, DMSO) δ 10.71 (d, J=17.9 Hz, 1H), 8.88-8.68 (m, 1H), 8.51-8.47 (m, 1H), 8.22 (d, J=15.6 Hz, 1H), 7.83 (d, J=27.6 Hz, 1H), 7.37 (d, J=11.2 Hz, 1H), 7.17 (d, J=12.8 Hz, 1H), 4.95-4.71 (m, 4H), 3.16 (d, J=26.3 Hz, 3H), 2.96 (s, 4H), 2.56 (d, J=14.8 Hz, 1H), 2.39 (s, 3H), 2.03-1.87 (m, 1H), 1.55 (s, 2H), 0.97-0.90 (m, 2H), 0.67-0.61 (m, 2H).

### Example 669

### Synthesis (1S,2S)—N-(4-cyano-3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (I-669)

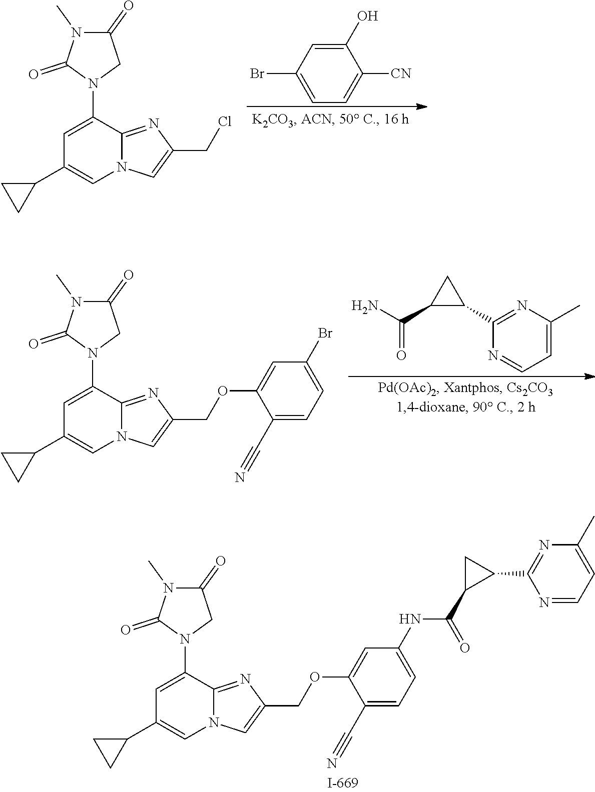 [SMILES: CN1C(=O)CN(c2cc(C3CC3)cn3cc(CCl)nc23)C1=O ; N#Cc1ccc(Br)cc1O ; CN1C(=O)CN(c2cc(C3CC3)cn3cc(COc4cc(Br)ccc4C#N)nc23)C1=O ; Cc1ccnc([C@H]2C[C@@H]2C(N)=O)n1 ; Cc1ccnc([C@H]2C[C@@H]2C(=O)Nc2ccc(C#N)c(OCc3cn4cc(C5CC5)cc(N5CC(=O)N(C)C5=O)c4n3)c2)n1]

Synthesis of 4-bromo-2-((6-cyclopropyl-8-(3-methyl-2,4-dioxoimidazolidin-1-yl)imidazo[1,2-a]pyridin-2-yl)methoxy)benzonitrile. To a solution of 1-(2-(chloromethyl)-6-cyclopropylimidazo[1,2-a]pyridin-8-yl)-3-methylimidazolidine-2,4-dione (0.11 g, 0.36 mmol) in ACN (5.0 mL) was added 4-bromo-2-hydroxybenzonitrile (0.14 g, 0.72 mmol) and K₂CO₃ (0.50 g, 3.6 mmol). After the mixture was stirred at 50° C. for 16 h, water (20 mL) was added and extracted with EtOAc (3×20 mL). The combined organic layers were washed with brine, dried over Na₂SO₄, filtered and concentrated in vacuo to give the crude, which was purified by preparative TLC to give 4-bromo-2-((6-cyclopropyl-8-(3-methyl-2,4-dioxoimidazolidin-1-yl)imidazo[1,2-a]pyridin-2-yl)methoxy)benzonitrile (0.10 g, 58%) as a white solid. ESI-MS [M+H]+: 480.1.

Synthesis of (1S,2S)—N-(4-cyano-3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide. To a solution of 4-bromo-2-((6-cyclopropyl-8-(3-methyl-2,4-dioxoimidazolidin-1-yl)imidazo[1,2-a]pyridin-2-yl)methoxy)benzonitrile (0.10 g, 0.21 mmol) in 1,4-dioxane (8.0 mL) was added (1S,2S)-2-(4-methylpyrimidin-2-yl)cyclopropane-1-carboxamide (39 mg, 0.22 mmol), Cs₂CO₃ (0.20 g, 0.63 mmol), Pd₂(dba)₃ (19 mg, 0.021 mmol), and Xantphos (24 mg, 0.042 mmol). The mixture was stirred at 90° C. for 2 h under N₂. The reaction mixture was filtered through Celite® and the filter cake was washed with DCM/MeOH (v/v=10/1, 30 mL). The filtrate was concentrated in vacuo to give the crude, which was purified by prep-HPLC to give (1S,2S)—N-(4-cyano-3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide (21 mg, 17%) as a white solid. ESI-MS [M+H]⁺: 577.2. ¹H NMR (400 MHz, DMSO) δ 10.77 (s, 1H), 8.53 (d, J=4.9 Hz, 1H), 8.31 (s, 1H), 7.94 (d, J=22.7 Hz, 2H), 7.63 (d, J=8.5 Hz, 1H), 7.40 (s, 1H), 7.21-7.19 (m, 2H), 5.32 (s, 2H), 4.93 (d, J=18.4 Hz, 2H), 2.97 (s, 3H), 2.53-2.51 (m, 2H), 2.42 (s, 3H), 1.97-1.94 (m, 1H), 1.62-1.48 (m, 2H), 0.96-0.94 (m, 2H), 0.68-0.66 (m, 2H).

### Example 670

### Synthesis of (1S,2S)—N-(5-((6-cyclopropyl-8-(3-methyl-2,4-dioxoimidazolidin-1-yl)imidazo[1,2-a]pyridin-2-yl)methoxy)-6-(methylamino)pyridazin-3-yl)-2-(4-methylpyrimidin-2-yl)cyclopropane-1-carboxamide (I-670)

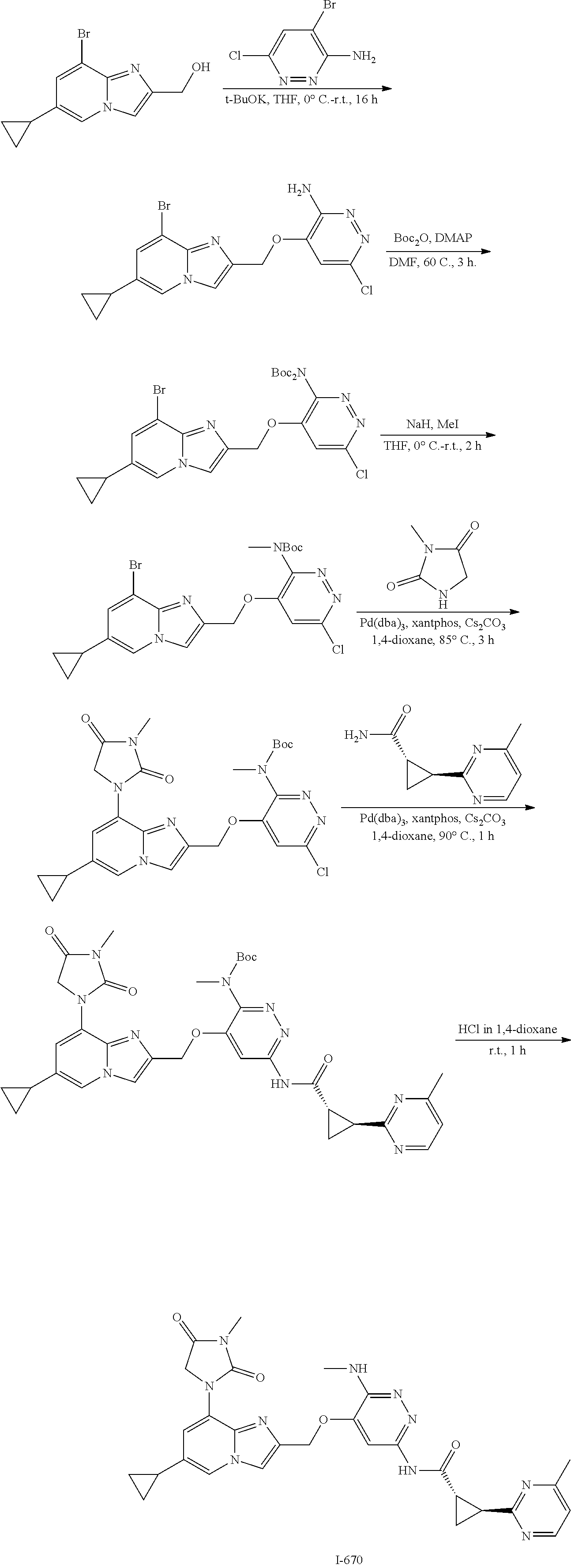

Synthesis of 4-((8-bromo-6-cyclopropylimidazo[1,2-a]pyridin-2-yl)methoxy)-6-chloropyridazin-3-amine. To mixture of (8-bromo-6-cyclopropylimidazo[1,2-a]pyridin-2-yl)methanol (1.1 g, 4.0 mmol) and 4-bromo-6-chloropyridazin-3-amine (1.3 g, 6.0 mmol) in dry THF (40 mL) was added t-BuOK (1M in THF, 12 mL) at 0° C. After stirring at room temperature for 16 h, the reaction was quenched with saturated aq·NH₄Cl (20 mL) and extracted with EtOAc (3×30 mL×3). The combined organic layers were washed with brine (30 mL), dried over anhydrous Na₂SO₄, and concentrated in vacuo to give the crude, which was purified by column chromatography on silica gel (eluent: DCM/MeOH=30/1) to give 4-((8-bromo-6-cyclopropylimidazo[1,2-a]pyridine-2-yl)methoxy)-6-chloropyridazin-3-amine (1.5 g, 95%) as a yellow solid. ESI-MS [M+H]⁺: 396.2.

Synthesis of tert-butyl (4-((8-bromo-6-cyclopropylimidazo[1,2-a]pyridin-2-yl)methoxy)-6-chloropyridazin-3-yl)(tert-butoxycarbonyl)carbamate. A mixture of 4-((8-bromo-6-cyclopropylimidazo[1,2-a]pyridin-2-yl)methoxy)-6-chloropyridazin-3-amine (1.5 g, 3.8 mmol), Boc₂O (2.4 g, 11 mmol) and DMAP (0.46 g, 3.8 mmol) in DMF (30 mL) was stirred at 60° C. for 3 h. The reaction was quenched with water (50 mL) and extracted with EtOAc (3×30 mL). The combined organic layers were washed with brine (50 mL), dried over anhydrous Na₂SO₄, and concentrated in vacuo to give the crude, which was purified by column chromatography on silica gel (eluent: DCM/MeOH=50/1) to give tert-butyl (4-((8-bromo-6-cyclopropylimidazo[1,2-a]pyridin-2-yl)methoxy)-6-chloropyridazin-3-yl)(tert-butoxycarbonyl)carbamate (1.5 g, 66%) as a yellow solid. ESI-MS [M+H]⁺: 596.2.

Synthesis of tert-butyl (4-((8-bromo-6-cyclopropylimidazo[1,2-a]pyridin-2-yl)methoxy)-6-chloropyridazin-3-yl)(methyl)carbamate. To mixture of tert-butyl (4-((8-bromo-6-cyclopropylimidazo[1,2-a]pyridin-2-yl)methoxy)-6-chloro pyridazin-3-yl)(tert-butoxycarbonyl)carbamate (0.50 g, 0.84 mmol) in dry THF (15 mL) was added NaH (0.10 g, 2.5 mmol) at 0° C. for 0.5 h. Then a solution of Mel (0.36 g, 2.5 mmol) in dry THF (5.0 mL) was added and it was stirred at room temperature for another 2 h. The reaction was quenched with saturated aq·NH₄Cl (20 mL) and extracted with EtOAc (3×30 mL). The combined organic layers were washed with brine (30 mL), dried over anhydrous Na₂SO₄, and concentrated in vacuo to give the crude, which was purified by column chromatography on silica gel (eluent: DCM/MeOH=20/1) to give tert-butyl (4-((8-bromo-6-cyclopropylimidazo[1,2-a]pyridin-2-yl)methoxy)-6-chloropyridazin-3-yl)(methyl)carbamate (0.25 g, 59%) as a yellow solid. ESI-MS [M+H]⁺: 510.1.

Synthesis of tert-butyl (6-chloro-4-((6-cyclopropyl-8-(3-methyl-2,4-dioxoimidazolidin-1-yl)imidazo[1,2-a]pyridin-2-yl)methoxy)pyridazin-3-yl)(methyl)carbamate. To a mixture of tert-butyl (4-((8-bromo-6-cyclopropylimidazo[1,2-a]pyridin-2-yl)methoxy)-6-chloropyridazin-3-yl)(methyl)carbamate (0.20 g, 0.40 mmol), 3-methylimidazolidine-2,4-dione (46 mg, 0.40 mmol), and Cs₂CO₃ (0.39 g, 1.2 mmol) in 1,4-dioxane (10 mL) were added Pd₂(dba)₃ (37 mg, 0.040 mmol) and Xantphos (46 mg, 0.080 mmol). The reaction mixture was stirred at 85° C. for 3 h under N₂ and cooled to room temperature. The reaction mixture was filtered through Celite® and the filter cake was washed with DCM/MeOH (v/v=10/1, 30 mL). The filtrate was concentrated in vacuo to give the crude, which was purified by preparative TLC (eluent: DCM/MeOH=20/1) to give tert-butyl (6-chloro-4-((6-cyclopropyl-8-(3-methyl-2,4-dioxoimidazolidin-1-yl)imidazo[1,2-a]pyridin-2-yl)methoxy)pyridazin-3-yl)(methyl)carbamate (80 mg, 37%) as a yellow solid. ESI-MS [M+H]⁺: 542.2.

Synthesis of tert-butyl (4-((6-cyclopropyl-8-(3-methyl-2,4-dioxoimidazolidin-1-yl)-imidazo[1,2-a]pyridin-2-yl)methoxy)-6-((1S,2S)-2-(4-methylpyrimidin-2-yl)cyclopropane-1-carboxamido)pyridazin-3-yl)(methyl)carbamate. To a mixture of tert-butyl (6-chloro-4-((6-cyclopropyl-8-(3-methyl-2,4-dioxoimidazolidin-1-yl) imidazo[1,2-a]pyridin-2-yl)methoxy)pyridazin-3-yl)(methyl)carbamate (0.12 g, 0.22 mmol), (1S,2S)-2-(4-methylpyrimidin-2-yl)cyclopropane-1-carboxamide (40 mg, 0.22 mmol), and Cs₂CO₃ (0.22 g, 0.66 mmol) in 1,4-dioxane (10 mL) were added Pd₂(dba)₃ (20 mg, 0.022 mmol) and Xantphos (25 mg, 0.044 mmol). After stirring at 90° C. for 1 h under N₂, the reaction mixture was cooled to room temperature and filtered through Celite®. The filter cake was washed with DCM/MeOH (v/v=10/1, 30 mL) and the filtrate was concentrated in vacuo to give the crude, which was purified by preparative TLC (eluent: DCM/MeOH=20/1) to give tert-butyl (4-((6-cyclopropyl-8-(3-methyl-2,4-dioxoimidazolidin-1-yl)imidazo[1,2-a]pyridin-2-yl)methoxy)-6-((1S,2S)-2-(4-methylpyrimidin-2-yl)cyclopropane-1-carboxamido)pyridazin-3-yl)(methyl)carbamate (40 mg, 27%) as a yellow solid. ESI-MS [M+H]⁺: 683.2.

Synthesis of (1S,2S)—N-(5-((6-cyclopropyl-8-(3-methyl-2,4-dioxoimidazolidin-1-yl)imidazo[1,2-a]pyridin-2-yl)methoxy)-6-(methylamino)pyridazin-3-yl)-2-(4-methylpyrimidin-2-yl)cyclopropane-1-carboxamide (I-670). A mixture of tert-butyl (4-((6-cyclopropyl-8-(3-methyl-2,4-dioxoimidazolidin-1-yl)imidazo[1,2-a] pyridin-2-yl)methoxy)-6-((1S,2S)-2-(4-methylpyrimidin-2-yl)cyclopropane-1-carboxamido)pyridazin-3-yl)(methyl)carbamate (40 mg, 0.060 mmol) in HCl (4M solution in 1,4-dioxane, 5.0 mL) was stirred at room temperature for 1 h. The reaction mixture was concentrated in vacuo to give the crude, which was purified by preparative HPLC to give (1S,2S)—N-(5-((6-cyclopropyl-8-(3-methyl-2,4-dioxoimidazolidin-1-yl)imidazo[1,2-a]pyridin-2-yl)methoxy)-6-(methylamino)pyridazin-3-yl)-2-(4-methylpyrimidin-2-yl)cyclopropane-1-carboxamide (12 mg, 35%) as a white solid. ESI-MS [M+H]⁺: 583.2. ¹H NMR (400 MHz, DMSO) δ 10.82 (s, 1H), 8.53 (d, J=5.1 Hz, 1H), 8.29 (s, 1H), 8.05 (s, 1H), 7.87 (s, 1H), 7.40 (d, J=1.4 Hz, 1H), 7.21 (d, J=5.1 Hz, 1H), 6.17 (q, J=4.8 Hz, 1H), 5.31 (s, 2H), 4.94 (d, J=4.0 Hz, 2H), 2.98 (s, 3H), 2.85 (d, J=4.8 Hz, 3H), 2.57-2.54 (m, 2H), 2.42 (s, 3H), 2.00-1.95 (m, 1H), 1.53-1.49 (m, 2H), 1.03-0.86 (m, 2H), 0.73-0.63 (m, 2H).

### Example 671

### Synthesis of (1S,2S)—N-(5-((6-cyclopropyl-8-(3-methyl-2,4-dioxoimidazolidin-1-yl)imidazo[1,2-a]pyridin-2-yl)methoxy)-1,2,4-triazin-3-yl)-2-(4-methylpyrimidin-2-yl)cyclopropane-1-carboxamide (I-671)

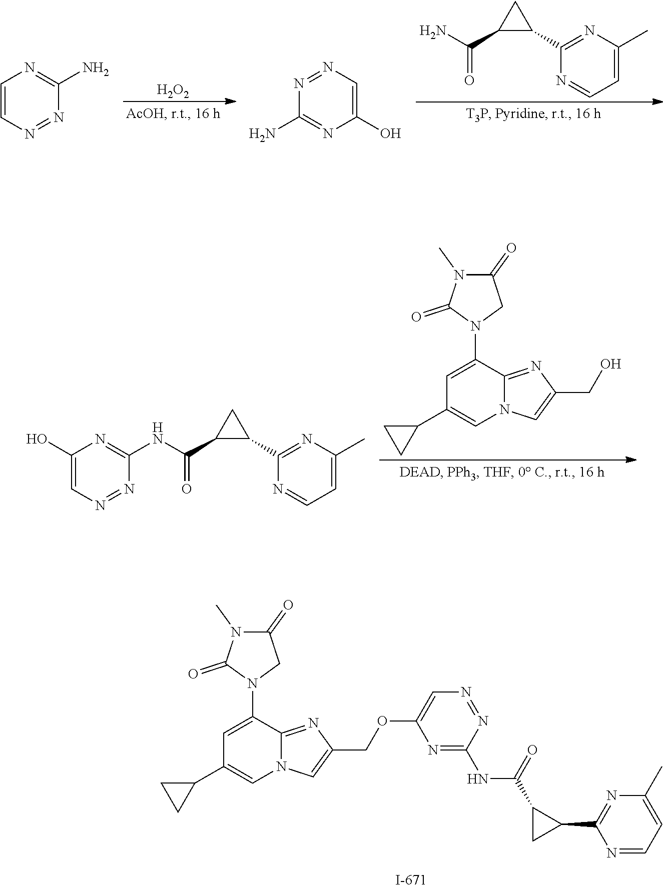

Synthesis of 3-amino-1,2,4-triazin-5-ol. To a mixture of 1,2,4-triazin-3-amine (2.4 g, 25 mmol) in acetic acid (15 mL) was added dropwise H₂O₂ (30% w/w in H₂O, 2.5 mL, 50 mmol) at room temperature. After stirring for 16 h, the mixture was diluted with EtOAc (40 mL) and filtered. The filter cake was washed with EtOAc (2×30 mL) and then added MeOH (40 mL). The resulting solution was concentrated in vacuo to afford 3-amino-1,2,4-triazin-5-ol (2 g, 71%) as a white solid. ESI-MS [M+H]⁺: 113.1.

Synthesis of (1S,2S)—N-(5-hydroxy-1,2,4-triazin-3-yl)-2-(4-methylpyrimidin-2-yl)cyclopropane-1-carboxamide. To a reaction mixture of 3-amino-1,2,4-triazin-5-ol (0.67 g, 6.0 mmol) and (1S,2S)-2-(4-methylpyrimidin-2-yl)cyclopropane-1-carboxylic acid (0.59 g, 5.0 mmol) in pyridine (9.0 mL) was added T₃P (4.7 g, 15 mmol). After stirring at room temperature for 16 h, the reaction mixture was diluted with water (50 mL) and extracted with EtOAc (50 mL). The organic phase was washed with brine, dried over Na₂SO₄, filtered and concentrated in vacuo and the residue was purified by column chromatography on silica gel (eluent: DCM/MeOH=15/1) to give (1S,2S)—N-(5-hydroxy-1,2,4-triazin-3-yl)-2-(4-methylpyrimidin-2-yl)cyclopropane-1-carboxamide (0.70 g, 78%) as yellow solid. ESI-MS [M+H]⁺: 273.1.

Synthesis of (1S,2S)—N-(5-((6-cyclopropyl-8-(3-methyl-2,4-dioxoimidazolidin-1-yl)imidazo[1,2-a]pyridin-2-yl)methoxy)-1,2,4-triazin-3-yl)-2-(4-methylpyrimidin-2-yl)cyclopropane-1-carboxamide (I-671). To a mixture of (1S,2S)—N-(5-hydroxy-1,2,4-triazin-3-yl)-2-(4-methylpyrimidin-2-yl)cyclopropane-1-carboxamide (54 mg, 0.20 mmol) and PPh₃ (0.16 g, 0.60 mol) in THF (10 mL) at 0° C. was added 1-(6-cyclopropyl-2-(hydroxymethyl)imidazo[1,2-a]pyridin-8-yl)-3-methylimidazolidine-2,4-dione (90 mg, 0.30 mmol), followed by DEAD (0.14 g, 0.80 mmol). After stirring the mixture at room temperature under N₂ for 16 hours, the reaction was concentrated in vacuo to give the crude, which was purified with preparative TLC (eluent: DCM/MeOH=18/1) to give (1S,2S)—N-(5-((6-cyclopropyl-8-(3-methyl-2,4-dioxoimidazolidin-1-yl)imidazo[1,2-a]pyridin-2-yl)methoxy)-1,2,4-triazin-3-yl)-2-(4-methylpyrimidin-2-yl)cyclopropane-1-carboxamide (15 mg, 14%) as yellow oil. ESI-MS [M+H]⁺: 555, ¹H NMR (400 MHz, DMSO) δ 11.35 (s, 1H), 8.81 (s, 1H), 8.52 (d, J=5.1 Hz, 1H), 8.29 (s, 1H), 8.08 (s, 1H), 7.39 (d, J=1.4 Hz, 1H), 7.19 (d, J=5.1 Hz, 1H), 5.49 (s, 2H), 4.88 (s, 2H), 2.97 (s, 3H), 2.90-2.88 (m, 1H), 2.63-2.60 (m, 1H), 2.40 (s, 3H), 1.99-1.94 (m, 1H), 1.63-1.59 (m, 2H), 0.97-0.94 (m, 2H), 0.69-0.65 (m, 2H).

### Example 672

### Synthesis of (1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide (I-672)

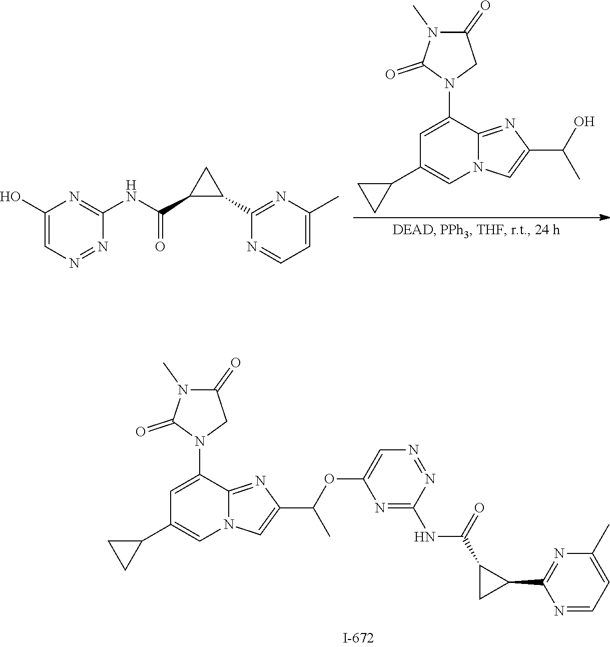

To a reaction mixture of (1S,2S)—N-(5-hydroxy-1,2,4-triazin-3-yl)-2-(4-methylpyrimidin-2-yl)-cyclopropane-1-carboxamide (54 mg, 0.20 mmol) and PPh₃ (0.16 g, 0.60 mmol) in THF (10 mL) was added 1-(6-cyclopropyl-2-(1-hydroxyethyl)imidazo[1,2-a]pyridin-8-yl)-3-methylimidazolidine-2,4-dione (94 mg, 0.30 mmol) at 0° C. Then DEAD (0.14 g, 0.80 mmol) was added dropwise and the solution was stirred at room temperature under N₂ for 24 hours. The mixture was concentrated in vacuo to give the crude which was purified with preparative TLC (eluent: DCM/MeOH=18/1) to give (1S,2S)—N-(5-(1-(6-cyclopropyl-8-(3-methyl-2,4-dioxoimidazolidin-1-yl)imidazo[1,2-a]pyridin-2-yl)ethoxy)-1,2,4-triazin-3-yl)-2-(4-methylpyrimidin-2-yl)cyclopropane-1-carboxamide (24 mg, 21%) as yellow oil. ESI-MS [M+H]+: 569.1 ¹H NMR (400 MHz, DMSO) δ 11.32 (s, 1H), 8.78 (s, 1H), 8.55-8.50 (m, 1H), 8.24-8.20 (m, 1H), 8.02 (d, J=18.5 Hz, 1H), 7.41-7.36 (m, 1H), 7.22-7.19 (m, 1H), 6.42-6.38 (m, 1H), 4.95-4.84 (m, 2H), 2.97 (s, 3H), 2.85-2.83 (m, 1H), 2.65-2.59 (m, 1H), 2.41 (d, J=5.6 Hz, 3H), 2.00-1.93 (m, 1H), 1.77-1.73 (m, 3H), 1.63-1.58 (m, 2H), 0.98-0.92 (m, 2H), 0.69-0.63 (m, 2H).

### Examples 673-674

Synthesis of 2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (I-673) and N-(4-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide (I-674)

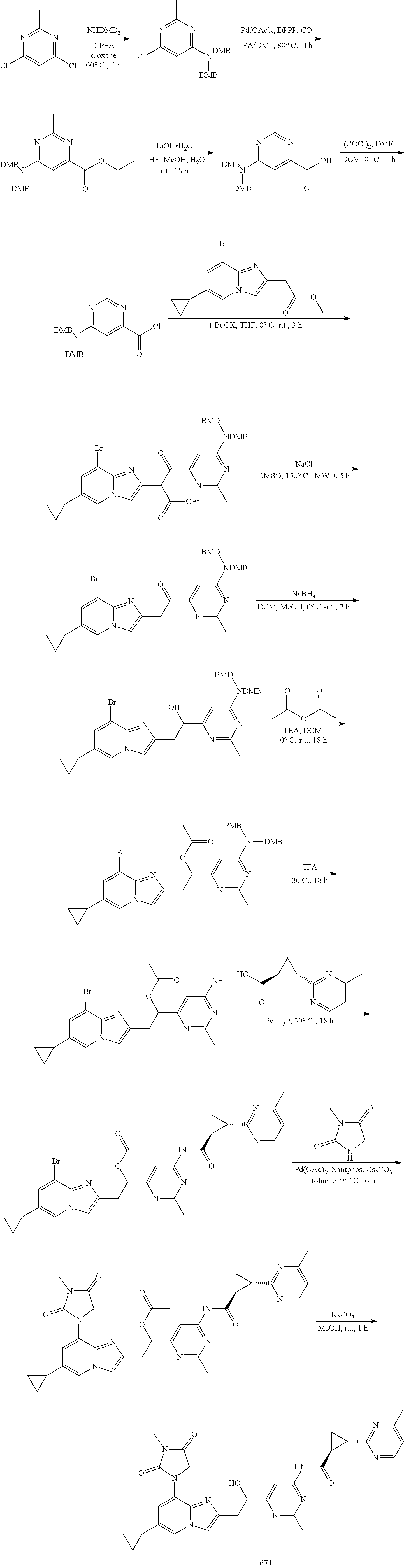

Synthesis of 6-chloro-N,N-bis(2,4-dimethoxybenzyl)-2-methylpyrimidin-4-amine. A mixture of 4,6-dichloro-2-methylpyrimidine (15 g, 93 mmol), bis(2,4-dimethoxybenzyl)amine (35 g, 0.11 mol), and DIPEA (36 g, 0.28 mol) in 1,4-dioxane (0.40 L) was stirred at 60° C. for 4 h. The mixture was quenched with saturated aq·NaHCO₃ (500 mL) and extracted with EtOAc (3×500 mL). The combined organic layers were washed with brine (500 mL), dried over Na₂SO₄ and concentrated in vacuo. The residue was purified by column chromatography on silica gel (eluent: EtOAc/PE=0-30%) to afford 6-chloro-N,N-bis(2,4-dimethoxybenzyl)-2-methylpyrimidin-4-amine (30 g, 73%) as an off-white solid. ESI-MS [M+H]+: 444.2

Synthesis of isopropyl 6-(bis(2,4-dimethoxybenzyl)amino)-2-methylpyrimidine-4-carboxylate. A mixture of 6-chloro-N,N-bis(2,4-dimethoxybenzyl)-2-methylpyrimidin-4-amine (10 g, 23 mmol), 1,3-Bis(diphenylphosphino)propane (0.95 g, 2.3 mmol), 1,3-Bis(diphenylphosphino)propane (0.95 g, 2.3 mmol), and Pd(OAc)₂ (0.52 g, 2.3 mmol) in IPA/DMF (10 mL/100 mL) was stirred at 80° C. for 4 h under CO. The mixture was quenched with water (1.0 L) and extracted with EtOAc (3×300 mL). The combined organic layers were washed with brine (300 mL), dried over Na₂SO₄ and concentrated in vacuo. The residue was purified by column chromatography on silica gel (eluent: EtOAc/PE=0-30%) to afford isopropyl 6-(bis(2,4-dimethoxybenzyl)amino)-2-methylpyrimidine-4-carboxylate (21 g, 52% yield, for 3 batches) as an off-white solid. ESI-MS [M+H]+: 496.2

Synthesis of 6-(bis(2,4-dimethoxybenzyl)amino)-2-methylpyrimidine-4-carboxylic acid. A mixture of isopropyl 6-(bis(2,4-dimethoxybenzyl)amino)-2-methylpyrimidine-4-carboxylate (10.0 g, 20.2 mmol) and LiOH·H₂O (1.7 g, 40 mmol) in THF/MeOH/H₂O (100 mL/100 mL/100 mL) was stirred in at room temperature for 18 h. The mixture was adjusted to pH=3 and extracted with IPA/CHCl₃ (5×300 mL). The combined organic layers were washed with brine (300 mL), dried over Na₂SO₄ and concentrated in vacuo to give 6-(bis(2,4-dimethoxybenzyl)amino)-2-methylpyrimidine-4-carboxylic acid (9.0 g, crude) as a white solid, which was used in the next step directly. ESI-MS [M+H]+: 454.2

Synthesis of 6-(bis(2,4-dimethoxybenzyl)amino)-2-methylpyrimidine-4-carbonyl chloride. To a mixture of 6-(bis(2,4-dimethoxybenzyl)amino)-2-methylpyrimidine-4-carboxylic acid (2.3 g, 5.0 mmol and oxalyl chloride (1.3 g, 10 mmol) in DCM (20 mL) was added DMF (one drop) and the mixture was stirred at room temperature for 1 h. The mixture was concentrated in vacuo to give 6-(bis(2,4-dimethoxybenzyl)amino)-2-methylpyrimidine-4-carbonyl chloride (2.5 g, crude) as a yellow solid, which was used in the next step without purification.

Synthesis of ethyl 3-(6-(bis(2,4-dimethoxybenzyl)amino)-2-methylpyrimidin-4-yl)-2-(8-bromo-6-cyclopropylimidazo[1,2-a]pyridin-2-yl)-3-oxopropanoate. To a mixture of ethyl 2-(8-bromo-6-cyclopropylimidazo[1,2-a]pyridin-2-yl)acetate (1.6 g, 5.0 mmol) in THF (30 mL) was added t-BuOK (16 mL, 16 mmol) at 0° C. and stirred at room temperature for 0.5 h. 6-(Bis(2,4-dimethoxybenzyl)amino)-2-methylpyrimidine-4-carbonyl chloride (2.5 g, 5.0 mmol) in THF (20 mL) was then added at 0° C. and the mixture was stirred at room temperature for 2.5 h. The reaction was quenched with saturated aq·NH₄Cl (100 mL) and extracted with EtOAc (3×50 mL). The combined organic layers were washed with brine (50 mL), dried over Na₂SO₄ and concentrated in vacuo to afford ethyl 3-(6-(bis(2,4-dimethoxybenzyl)amino)-2-methylpyrimidin-4-yl)-2-(8-bromo-6-cyclopropylimidazo[1,2-a]pyridin-2-yl)-3-oxopropanoate (3.6 g, crude) as a brown solid which was used in next step directly. ESI-MS [M+H]+: 758.2

Synthesis of 6-(bis(2,4-dimethoxybenzyl)amino)-2-methylpyrimidine-4-carbonyl chloride. A mixture of 3-(6-(bis(2,4-dimethoxybenzyl)amino)-2-methylpyrimidin-4-yl)-2-(8-bromo-6-cyclopropylimidazo[1,2-a]pyridin-2-yl)-3-oxopropanoate (0.5 g, 0.66 mmol) and NaCl (46 mg, 0.79 mmol) in DMSO/water (1.5 mL/0.10 mL) was stirred at 150° C. in a microwave reactor for 0.5 h. The mixture was quenched with water (7×200 mL) and extracted with EtOAc (3×50 mL). The combined organic layers were washed with brine (50 mL), dried over Na₂SO₄ and concentrated in vacuo. The residue was purified by column chromatography on silica gel (eluent: EtOAc/PE=0-50%) to afford 1-(6-(bis(2,4-dimethoxybenzyl)amino)-2-methylpyrimidin-4-yl)-2-(8-bromo-6-cyclopropylimidazo[1,2-a]pyridin-2-yl)ethan-1-one (1.0 g, 32%, for 7 batches) as a brown solid. ESI-MS [M+H]+: 686.2

Synthesis of 6-(bis(2,4-dimethoxybenzyl)amino)-2-methylpyrimidine-4-carbonyl chloride. To a mixture of 1-(6-(bis(2,4-dimethoxybenzyl)amino)-2-methylpyrimidin-4-yl)-2-(8-bromo-6-cyclopropylimidazo[1,2-a]pyridin-2-yl)ethan-1-one (0.40 g, 0.58 mmol) in DCM/MeOH (10 mL/1.0 mL) was added NaBH₄ (44 mg, 1.2 mmol) at 0° C. After stirring at room temperature for 2 h, the mixture was with poured into water (50 mL,) and extracted with EtOAc (3×30 mL). The combined organic layers were washed with brine (30 mL), dried over Na₂SO₄ and concentrated in vacuo. The residue was purified by column chromatography on silica gel (eluent: MeOH/DCM=0˜5%) concentrated in vacuo to give 1-(6-(bis(2,4-dimethoxybenzyl)amino)-2-methylpyrimidin-4-yl)-2-(8-bromo-6-cyclopropylimidazo[1,2-a]pyridin-2-yl)ethan-1-ol (0.36 g, 90%) as a white solid. ESI-MS [M+H]+: 688.2

Synthesis of 1-(6-(bis(2,4-dimethoxybenzyl)amino)-2-methylpyrimidin-4-yl)-2-(8-bromo-6-cyclopropylimidazo[1,2-a]pyridin-2-yl)ethyl acetate. To a mixture of 1-(6-(bis(2,4-dimethoxybenzyl)amino)-2-methylpyrimidin-4-yl)-2-(8-bromo-6-cyclopropylimidazo[1,2-a]pyridin-2-yl)ethan-1-ol (0.45 g, 0.66 mmol) and TEA (0.53 g, 5.3 mmol) in DCM (10 mL) was added acetic anhydride (0.34 g, 3.3 mmol) at 0° C. After stirring at room temperature for 18 h, the mixture was quenched with saturated aq·NaHCO₃ (50 mL) and extracted with EtOAc (3×50 mL). The combined organic layers were washed with brine (50 mL), dried over Na₂SO₄ and concentrated in vacuo. The residue was purified by column chromatography on silica gel (eluent: MeOH/DCM=0-3%) to give 1-(6-(bis(2,4-dimethoxybenzyl)amino)-2-methylpyrimidin-4-yl)-2-(8-bromo-6-cyclopropylimidazo[1,2-a]pyridin-2-yl)ethyl acetate (0.40 g, 83%) as a white solid. ESI-MS [M+H]+: 730.2

Synthesis of 1-(6-amino-2-methylpyrimidin-4-yl)-2-(8-bromo-6-cyclopropylimidazo[1,2-a]pyridin-2-yl)ethyl acetate. A mixture of 1-(6-(bis(2,4-dimethoxybenzyl)amino)-2-methylpyrimidin-4-yl)-2-(8-bromo-6-cyclopropylimidazo[1,2-a]pyridin-2-yl)ethyl acetate (0.42 g, 0.58 mmol) in TFA (5.0 mL) was stirred at 30° C. for 18 h. The mixture was quenched with saturated aq·NaHCO₃ (100 mL) and extracted with EtOAc (3×50 mL). The combined organic layers were washed with brine (30 mL), dried over Na₂SO₄ and concentrated in vacuo. The residue was purified by preparative TLC (eluent: MeOH/DCM=1/12) to afford 1-(6-amino-2-methylpyrimidin-4-yl)-2-(8-bromo-6-cyclopropylimidazo[1,2-a]pyridin-2-yl)ethyl acetate (0.20 g, 81%) as a white solid. ESI-MS [M+H]+: 430.1

Synthesis of 2-(8-bromo-6-cyclopropylimidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate. To a mixture of 1-(6-amino-2-methylpyrimidin-4-yl)-2-(8-bromo-6-cyclopropylimidazo[1,2-a]pyridin-2-yl)ethyl acetate (0.20 g, 0.47 mmol), (1S,2S)-2-(4-methylpyrimidin-2-yl)cyclopropane-1-carboxylic acid (0.12 g, 0.70 mmol) in pyridine (1.0 mL) was added T₃P (3.0 g, 4.7 mmol) and the mixture was degassed with N₂. After stirring at 30° C. for 18 h, the mixture was quenched with water (50 mL) and extracted with EtOAc (3×30 mL). The combined organic layers were washed with brine (30 mL), dried over Na₂SO₄ and concentrated in vacuo. The residue was purified by preparative TLC (eluent: MeOH/DCM=1/20) to afford 2-(8-bromo-6-cyclopropylimidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (0.18 g, 65%) as a white solid. ESI-MS [M+H]+: 590.1

Synthesis of 2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (I-673). A mixture of 2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (0.18 mg, 0.31 mmol), 3-methylimidazolidine-2,4-dione (0.11 mg, 0.92 mmol), Pd(OAc)₂ (14 mg, 0.062 mmol), Xantphos (53 mg, 0.092 mmol), and Cs₂CO₃ (0.20 mg, 0.62 mmol) in toluene (10 mL) was degassed with N₂ and stirred at 95° C. for 6 h. The mixture was quenched with water (50 mL) and extracted with EtOAc (3×30 mL). The combined organic layers were washed with brine (30 mL), dried over Na₂SO₄ and concentrated in vacuo. The residue was purified by preparative TLC (eluent: MeOH/DCM=1/20) to afford 2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (0.10 g, 53%) as a white solid. ESI-MS [M+H]+: 624.3. ¹H NMR (400 MHz, DMSO) δ 11.32 (s, 1H), 8.53 (d, J=5.1 Hz, 1H), 8.23 (s, 1H), 7.86 (s, 1H), 7.69 (s, 1H), 7.33 (d, J=1.2 Hz, 1H), 7.21 (d, J=5.0 Hz, 1H), 5.93-5.88 (m, 1H), 4.83 (s, 2H), 3.31-3.17 (m, 2H), 2.96 (s, 3H), 2.67-2.62 (m, 1H), 2.57 (s, 1H), 2.52 (s, 3H), 2.41 (s, 3H), 2.06 (s, 3H), 1.98-1.90 (m, 1H), 1.61-1.48 (m, 2H), 0.98-0.90 (m, 2H), 0.72-0.62 (m, 2H).

Synthesis of (1S,2S)—N-(6-(2-(6-cyclopropyl-8-(3-methyl-2,4-dioxoimidazolidin-1-yl)imidazo[1,2-a]pyridin-2-yl)-1-hydroxyethyl)-2-methylpyrimidin-4-yl)-2-(4-methylpyrimidin-2-yl)cyclopropane-1-carboxamide (I-674). A mixture of 2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate (60 mg, 0.096 mmol) and K₂CO₃ (27 mg, 0.19 mmol) in MeOH (5.0 mL) was stirred at room temperature for 1 h. The mixture was quenched with 1 N HCl (2.0 mL), then saturated aq·NaHCO₃ (50 mL) was added and the resulting mixture was extracted with EtOAc (3×30 mL). The combined organic layers were washed with brine (30 mL), dried over Na₂SO₄ and concentrated in vacuo. The residue was purified by preparative TLC (eluent: MeOH/DCM=1/20) to afford (1S,2S)—N-(6-(2-(6-cyclopropyl-8-(3-methyl-2,4-dioxoimidazolidin-1-yl)imidazo[1,2-a]pyridin-2-yl)-1-hydroxyethyl)-2-methylpyrimidin-4-yl)-2-(4-methylpyrimidin-2-yl)cyclopropane-1-carboxamide (25 mg, 45%) as a white solid.

ESI-MS [M+H]⁺: 582.0. ¹H NMR (400 MHz, DMSO) δ 11.20 (s, 1H), 8.57-8.50 (m, 1H), 8.25 (d, J=0.9 Hz, 1H), 8.08 (s, 1H), 7.67 (s, 1H), 7.30 (d, J=1.1 Hz, 1H), 7.22 (d, J=5.0 Hz, 1H), 5.64 (d, J=5.1 Hz, 1H), 4.92-4.79 (m, 3H), 3.23-3.17 (m, 1H), 2.99-2.95 (m, 3H), 2.94-2.86 (m, 1H), 2.69-2.63 (m, 1H), 2.59-2.54 (m, 1H), 2.52 (s, 3H), 2.42 (s, 3H), 1.99-1.90 (m, 1H), 1.60-1.49 (m, 2H), 1.01-0.87 (m, 2H), 0.71-0.58 (m, 2H).

### Example 675

### Synthesis of (1S,2S)—N-(6-((6-cyclopropyl-8-(3-methyl-2,4-dioxoimidazolidin-1-yl)imidazo[1,2-a]pyridin-2-yl)methoxy)-4-fluoropyridin-2-yl)-2-(4-methylpyrimidin-2-yl)cyclopropane-1-carboxamide (I-675)

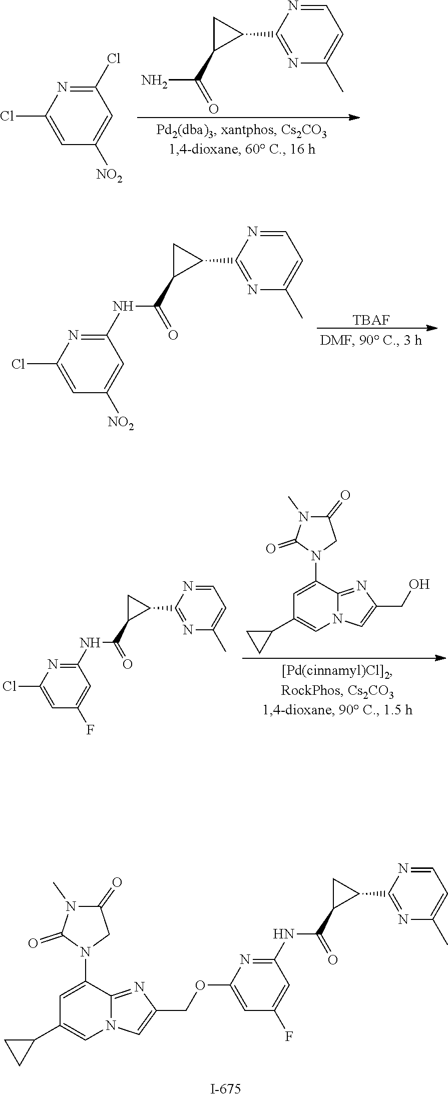

Synthesis of (1S,2S)—N-(6-chloro-4-nitropyridin-2-yl)-2-(4-methylpyrimidin-2-yl)cyclopropane-1-carboxamide. A mixture of 2,6-dichloro-4-nitropyridine (0.19 g, 1.0 mmol), (1S,2S)-2-(4-methylpyrimidin-2-yl)cyclopropane-1-carboxamide (0.18 g, 1.0 mmol), and Cs₂CO₃ (0.98 g, 3.0 mmol) in 1,4-dioxane (10 mL) were added Pd₂(dba)₃ (46 mg, 0.050 mmol) and Xantphos (58 mg, 0.10 mmol). After stirring at 60° C. for 16 h under N₂, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give (1S,2S)—N-(6-chloro-4-nitropyridin-2-yl)-2-(4-methylpyrimidin-2-yl) cyclopropane-1-carboxamide (80 mg, 24%) as a yellow solid. ESI-MS [M+H]+: 334.2.

Synthesis of (1S,2S)—N-(6-chloro-4-fluoropyridin-2-yl)-2-(4-methylpyrimidin-2-yl)cyclopropane-1-carboxamide. A mixture of (1S,2S)—N-(6-chloro-4-nitropyridin-2-yl)-2-(4-methylpyrimidin-2-yl)cyclopropane-1-carboxamide (0.10 g, 0.3 mmol) and TBAF (1M in THF, 0.75 mL) in DMF (5.0 mL) was stirred at 90° C. for 3 h under N₂. The reaction was quenched with water (30 mL) and extracted with EtOAc (3×30 mL). The combined organic layers were washed with brine (30 mL), dried over anhydrous Na₂SO₄, and concentrated in vacuo to give the crude product, which was purified by preparative TLC (eluent: DCM/MeOH=30/1) to give (1S,2S)—N-(6-chloro-4-fluoropyridin-2-yl)-2-(4-methylpyrimidin-2-yl)cyclopropane-1-carboxamide (55 mg, 60%) as a yellow solid. ESI-MS [M+H]⁺: 307.2.

Synthesis of (1S,2S)—N-(6-((6-cyclopropyl-8-(3-methyl-2,4-dioxoimidazolidin-1-yl)imidazo[1,2-a]pyridin-2-yl)methoxy)-4-fluoropyridin-2-yl)-2-(4-methylpyrimidin-2-yl)cyclopropane-1-carboxamide (I-675). To a mixture of (1S,2S)—N-(6-chloro-4-fluoropyridin-2-yl)-2-(4-methylpyrimidin-2-yl)cyclo propane-1-carboxamide (40 mg, 0.13 mmol), 1-(6-cyclopropyl-2-(hydroxymethyl)imidazo[1,2-a] pyridin-8-yl)-3-methylimidazolidine-2,4-dione (20 mg, 0.11 mmol), and Cs₂CO₃ (0.11 mg, 0.33 mmol) in 1,4-dioxane (8.0 mL) were added Di-chlorobis[(1,2,3-)-1-phenyl-2-propenyl] dipalladium(II) (7.0 mg, 0.013 mmol) and RockPhos (12 mg, 0.026 mmol). After stirring at 90° C. for 1.5 h under N₂, the reaction mixture was filtered through Celite® and the filter cake was washed with DCM/MeOH (10/1, 20 mL). The filtrate was concentrated in vacuo to give the crude, which was purified by preparative HPLC to give (1S,2S)—N-(6-((6-cyclopropyl-8-(3-methyl-2,4-dioxoimidazolidin-1-yl)imidazo[1,2-a]pyridin-2-yl)methoxy)-4-fluoropyridin-2-yl)-2-(4-methylpyrimidin-2-yl)cyclopropane-1-carboxamide (15 mg, 20%) as a white solid. ESI-MS [M+H]⁺: 571.2. ¹H NMR (400 MHz, DMSO) δ 11.02 (s, 1H), 8.54 (d, J=5.1 Hz, 1H), 8.42 (s, 1H), 8.10 (s, 1H), 7.63-7.39 (m, 2H), 7.23 (d, J=5.1 Hz, 1H), 6.51 (dd, J=9.6, 1.8 Hz, 1H), 5.47 (s, 2H), 4.80 (s, 2H), 2.97 (s, 3H), 2.62-2.52 (m, 2H), 2.43 (s, 3H), 2.06-1.92 (m, 1H), 1.56 (t, J=7.2 Hz, 2H), 0.99-0.96 (m, 2H), 0.72-0.69 (m, 2H).

### Example 676

### Synthesis of (1S,2S)—N-(5-((6-(1-fluorocyclopropyl)-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (I-676)

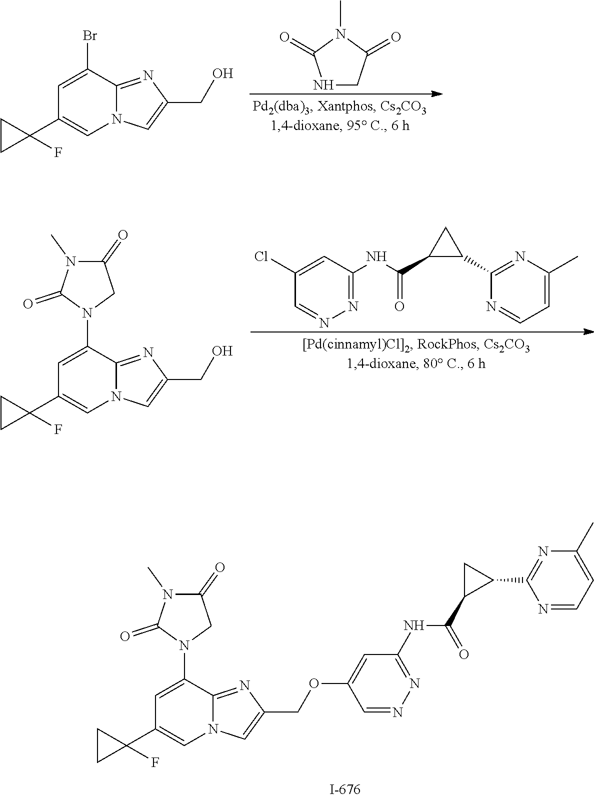

Synthesis of 1-(6-(1-fluorocyclopropyl)-2-(hydroxymethyl)imidazo[1,2-a]pyridin-8-yl)-3-methylimidazolidine-2,4-dione. To a stirred solution of (8-bromo-6-(1-fluorocyclopropyl)imidazo[1,2-a]pyridin-2-yl)methanol (86 mg, 0.30 mmol), 3-methylimidazolidine-2,4-dione (0.10 g, 0.90 mmol), Xantphos (35 mg, 0.060 mmol), and Cs₂CO₃ (0.29 g, 0.90 mmol) in 1,4-dioxane (10 mL) was added Pd₂(dba)₃ (23 mg, 0.025 mmol). Then the reaction was stirred at 95° C. for 6 h and cooled to room temperature. The mixture was then concentrated in vacuo to give the crude, which was purified by column chromatography on silica gel (eluent: MeOH/DCM=1/20) to give the 1-(6-(1-fluorocyclopropyl)-2-(hydroxymethyl)imidazo[1,2-a]pyridin-8-yl)-3-methylimidazolidine-2,4-dione (70 mg, 73%) as yellow solid. ESI-MS [M+H]⁺: 319.1.

Synthesis of (1S,2S)—N-(5-((6-(1-fluorocyclopropyl)-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (I-676). To a stirred solution of 1-(6-(1-fluorocyclopropyl)-2-(hydroxymethyl)imidazo[1,2-a]pyridin-8-yl)-3-methylimidazolidine-2,4-dione (64 mg, 0.20 mmol), (1S,2S)—N-(5-chloropyridazin-3-yl)-2-(4-methylpyrimidin-2-yl)cyclopropane-1-carboxamide (87 mg, 0.30 mmol), RockPhos (19 mg, 0.040 mmol), and Cs₂CO₃ (0.20 mg, 0.60 mmol) in 1,4-dioxane (10 mL) was added Di-chlorobis[(1,2,3-)-1-phenyl-2-propenyl]dipalladium(II) (10 mg, 0.020 mmol). The resulting mixture was stirred at 80° C. for 6 h and then concentrated in vacuo to give the crude, which was purified with silica gel chromatography (eluent: (MeOH:DCM=1:20) to give the (1S,2S)—N-(5-((6-(1-fluorocyclopropyl)-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide (6 mg, 5%) as white solid. ESI-MS [M+H]+: 572. ¹H NMR (400 MHz, DMSO) δ 11.38 (s, 1H), 8.78 (d, J=2.5 Hz, 1H), 8.59 (s, 1H), 8.54 (d, J=5.1 Hz, 1H), 8.21 (d, J=2.4 Hz, 1H), 8.12 (s, 1H), 7.66 (s, 1H), 7.22 (d, J=5.1 Hz, 1H), 5.41 (s, 2H), 5.01-4.92 (m, 2H), 2.99 (s, 3H), 2.69-2.67 (m, 1H), 2.58-2.55 (m, 1H), 2.42 (s, 3H), 1.57-1.45 (m, 4H), 1.16 (q, J=7.8 Hz, 2H).

### Example 677

### Synthesis of (1S,2S)—N-(5-((6-cyclopropyl-8-(3-methyl-2,4-dioxoimidazolidin-1-yl)imidazo[1,2-a]pyridin-2-yl)methoxy)-6-(hydroxymethyl)pyridazin-3-yl)-2-(4-methylpyrimidin-2-yl)cyclopropane-1-carboxamide (I-677)

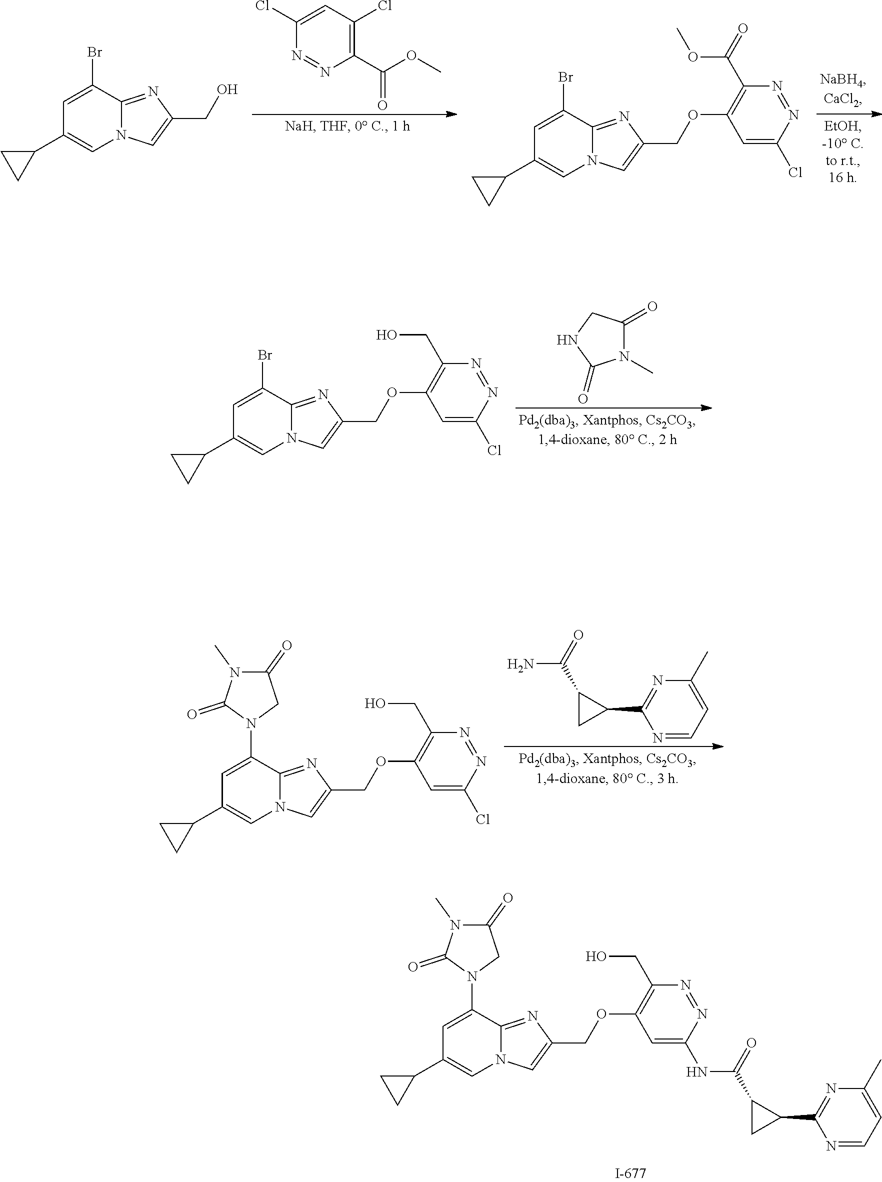

Synthesis of methyl 4-((8-bromo-6-cyclopropylimidazo[1,2-a]pyridin-2-yl)methoxy)-6-chloropyridazine-3-carboxylate. To a solution of (8-bromo-6-cyclopropylimidazo[1,2-a]pyridin-2-yl)methanol (1.2 g, 4.5 mmol) in dry THF (20.0 mL) was added NaH (360 mg, 60% in mineral oil, 9.0 mmol) at 0° C. After the reaction was stirred at 0° C. for 0.5 h, a solution of methyl 4,6-dichloropyridazine-3-carboxylate (1.38 g, 6.75 mmol) in dry THF (20.0 mL) was added and the resulting mixture was stirred at room temperature for another 2 h. The reaction was quenched with saturated aqueous NH₄Cl (50 mL) and extracted with EtOAc (50 mL×3). The combined organic layers were washed with brine (50 mL), dried over anhydrous Na₂SO₄, and concentrated in vacuo to give the crude, which was purified by column chromatography (eluent: DCM/MeOH=50/1) to give methyl 4-((8-bromo-6-cyclopropylimidazo[1,2-a]pyridin-2-yl)methoxy)-6-chloropyridazine-3-carboxylate (1.7 g, 86%) as a yellow solid. ESI-MS [M+H]⁺: 438.1.

Synthesis of (4-((8-bromo-6-cyclopropylimidazo[1,2-a]pyridin-2-yl)methoxy)-6-chloropyridazin-3-yl)methanol. To a mixture of methyl 4-((8-bromo-6-cyclopropylimidazo[1,2-a]pyridin-2-yl)methoxy)-6-chloropyridazine-3-carboxylate (1.3 g, 3.0 mmol) and CaCl₂ (660 mg, 6.0 mmol) in dry THF (40.0 mL) was added NaBH₄ (338 mg, 8.9 mmol) at −10° C. The resulting mixture was stirred at room temperature for 16 h. The reaction was quenched with saturated aqueous NH₄Cl (40 mL) and extracted with EtOAc (30 mL×3). The combined organic layers were washed with brine (30 mL), dried over anhydrous Na₂SO₄, and concentrated in vacuo to give the crude, which was purified by column chromatography (eluent: DCM/MeOH=20/1) to give (4-((8-bromo-6-cyclopropylimidazo[1,2-a]pyridin-2-yl)methoxy)-6-chloropyridazin-3-yl)methanol (900 mg, 73%) as a yellow solid. ESI-MS [M+H]⁺: 410.2.

Synthesis of 1-(2-(1-((6-chloro-3-methylpyridazin-4-yl)oxy)ethyl)-6-cyclopropylimidazo[1,2-a]pyridin-8-yl)-3-methylimidazolidine-2,4-dione. A mixture of (4-((8-bromo-6-cyclopropylimidazo[1,2-a]pyridin-2-yl)methoxy)-6-chloropyridazin-3-yl)methanol (410 mg, 1.0 mmol), 3-methylimidazolidine-2,4-dione (228 mg, 2.0 mmol), and Cs₂CO₃ (815 mg, 2.5 mmol) in 1,4-dioxane (20 mL) was added Pd₂(dba)₃ (46 mg, 0.05 mmol) and Xantphos (58 mg, 0.1 mmol). The reaction mixture was stirred at 80° C. for 2 h under N₂. The reaction mixture was filtered through Celite® and the filter cake was washed with DCM/MeOH (10/1, 30 mL). The filtrate was concentrated in vacuo to give the crude, which was purified by preparative HPLC to give 1-(2-(((6-chloro-3-(hydroxymethyl)pyridazin-4-yl)oxy)methyl)-6-cyclopropylimidazo[1,2-a]pyridin-8-yl)-3-methylimidazolidine-2,4-dione (70 mg, 16%) as a white solid. ESI-MS [M+H]⁺: 443.2.

Synthesis of (1S,2S)—N-(5-((6-cyclopropyl-8-(3-methyl-2,4-dioxoimidazolidin-1-yl)imidazo[1,2-a]pyridin-2-yl)methoxy)-6-(hydroxymethyl)pyridazin-3-yl)-2-(4-methylpyrimidin-2-yl)cyclopropane-1-carboxamide (I-677). To a mixture of 1-(2-(((6-chloro-3-(hydroxymethyl)pyridazin-4-yl)oxy)methyl)-6-cyclopropylimidazo[1,2-a]pyridin-8-yl)-3-methylimidazolidine-2,4-dione (70 mg, 0.16 mmol), (1S,2S)-2-(4-methylpyrimidin-2-yl)cyclopropane-1-carboxamide (28 mg, 0.16 mmol), and Cs₂CO₃ (129 mg, 0.39 mmol) in 1,4-dioxane (8 mL) was added Pd₂(dba)₃ (14.6 mg, 0.016 mmol) and Xantphos (18.3 mg, 0.032 mmol). The reaction mixture was stirred at 80° C. for 3 h under N₂. The reaction mixture was filtered through Celite® and the filter cake was washed with DCM/MeOH (10/1, 20 mL). The filtrate was concentrated in vacuo to give the crude, which was purified by preparative TLC to give (1S,2S)—N-(5-((6-cyclopropyl-8-(3-methyl-2,4-dioxoimidazolidin-1-yl)imidazo[1,2-a]pyridin-2-yl)methoxy)-6-(hydroxymethyl)pyridazin-3-yl)-2-(4-methylpyrimidin-2-yl)cyclopropane-1-carboxamide (16 mg, 17%) as a white solid. ESI-MS [M+H]⁺: 584.2. ¹H NMR (400 MHz, DMSO) δ 11.38 (s, 1H), 8.54 (d, J=5.1 Hz, 1H), 8.30 (s, 2H), 8.01 (s, 1H), 7.41 (s, 1H), 7.22 (d, J=5.1 Hz, 1H), 5.38 (s, 2H), 5.16 (t, J=5.8 Hz, 1H), 5.03-4.82 (m, 2H), 4.63 (d, J=3.9 Hz, 2H), 2.99 (s, 3H), 2.56-2.54 (m, 2H), 2.42 (s, 3H), 2.00-1.94 (m, 1H), 1.56 (t, J=7.2 Hz, 2H), 1.03-0.87 (m, 2H), 0.69-0.65 (m, 2H).

### Example 678

Synthesis of (1S,2S)—N-(6-(((R)-1-(6-(fluoromethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-678)

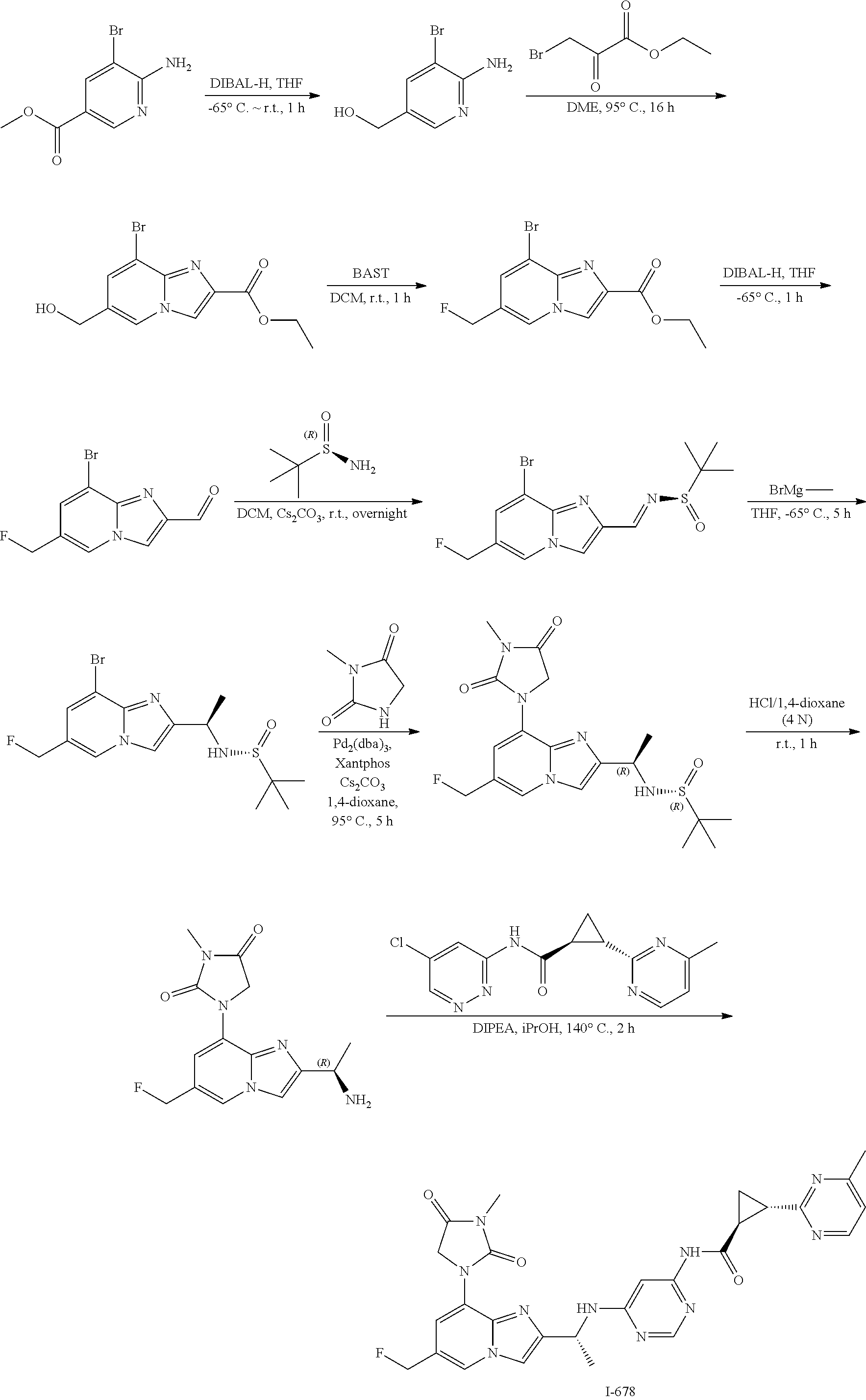

Synthesis of (6-amino-5-bromopyridin-3-yl)methanol. To a solution of methyl 6-amino-5-bromonicotinate (3.0 g, 13 mmol) in THF (30 mL) was added DIBAL-H (36 mL, 1.0 N in hexane, 36 mmol) dropwise at −65° C. After stirring at −65° C. for 0.5 h, the mixture was warmed to room temperature and stirred for another 0.5 h. The reaction mixture was quenched with NaOH (1.0 M aq·, 36 mL) and then extracted with EtOAc (50 mL×3). The combined organics were washed with brine (100 mL), dried over Na₂SO₄, and concentrated in vacuo. The residue was purified by column chromatography on silica gel, eluting with 0˜20% EtOAc in PE to give (6-amino-5-bromopyridin-3-yl)methanol (2.4 g, yield: 91%) as a white solid. ESI-MS [M+H]⁺: 203.0.

Synthesis of ethyl 8-bromo-6-(hydroxymethyl)imidazo[1,2-a]pyridine-2-carboxylate. A mixture of ethyl 3-bromo-2-oxopropanoate (3.3 g, 17 mmol) and (6-amino-5-bromopyridin-3-yl)methanol (2.4 g, 12 mmol) in DME (50 mL) was stirred at 95° C. for 24 h. The mixture was cooled to room temperature and concentrated in vacuo. The residue was diluted with NaHCO₃ (sat·aq·, 50 mL) and then extracted with DCM (50 mL×3). The combined organic layers were washed with brine (30 mL), dried over Na₂SO₄, and concentrated in vacuo. The residue was purified by column chromatography on silica gel, eluting with 0˜40% EtOAc in PE to give ethyl 8-bromo-6-(hydroxymethyl)imidazo[1,2-a]pyridine-2-carboxylate (1.0 g, yield: 28%) as a brown solid. ESI-MS [M+H]+: 299.0

Synthesis of ethyl 8-bromo-6-(fluoromethyl)imidazo[1,2-a]pyridine-2-carboxylate. To a solution of ethyl 8-bromo-6-(hydroxymethyl)imidazo[1,2-a]pyridine-2-carboxylate (1.0 g, 3.3 mmol) in DME (20 mL) was added BAST (1.8 mL, 10 mmol) at 0° C. After stirring at room temperature for 12 h, the reaction mixture was quenched with NaHCO₃ (Sat·aq·, 50 mL) and extracted with DCM (50 mL×3). The combined organics were washed with brine (30 mL), dried over Na₂SO₄, and concentrated in vacuo. The residue was purified by column chromatography on silica gel, eluting with 10˜20% EtOAc in PE to give ethyl 8-bromo-6-(fluoromethyl)imidazo[1,2-a]pyridine-2-carboxylate (0.55 g, yield: 55%) as a white solid. ESI-MS [M+H]+: 301.0

Synthesis of 8-bromo-6-(fluoromethyl)imidazo[1,2-a]pyridine-2-carbaldehyde. To a solution of ethyl 8-bromo-6-(fluoromethyl)imidazo[1,2-a]pyridine-2-carboxylate (0.55 g, 1.8 mmol) in THF (20 mL) was added DIBAL-H (5.4 mL, 1.0 M in hexane, 5.4 mmol) at −65° C. After stirring at −65° C. for 1 h, the reaction mixture was quenched by adding sodium sulfate decahydrate at −65° C., warmed to 0° C. and stirred for 10 min. The reaction mixture was filtered and the filtrate was concentrated to give residue, which was purified by column chromatography on silica gel, eluting with 0˜50% EtOAc in PE to give 8-bromo-6-(fluoromethyl)imidazo[1,2-a]pyridine-2-carbaldehyde (0.30 g, yield: 65%) as a white solid. ESI-MS [M+H]⁺: 257.1.

Synthesis of (R,E)-N-((8-bromo-6-(fluoromethyl)imidazo[1,2-a]pyridin-2-yl)methylene)-2-methylpropane-2-sulfinamide. A mixture of 8-bromo-6-(fluoromethyl)imidazo[1,2-a]pyridine-2-carbaldehyde (0.30 g, 1.2 mmol), (R)-2-methylpropane-2-sulfinamide (0.17 g, 1.4 mmol) and Cs₂CO₃ (0.78 g, 2.4 mmol) in DCM (25 mL) was stirred at room temperature overnight. The resulting mixture was filtered and the filtrate was concentrated in vacuo. The residue was purified by column chromatography on silica gel, eluting with 0˜50% EtOAc in PE to give (R,E)-N-((8-bromo-6-(fluoromethyl)imidazo[1,2-a]pyridin-2-yl)methylene)-2-methylpropane-2-sulfinamide (0.30 g, yield: 69%) as a pale-yellow solid. ESI-MS [M+H]+: 360.1

Synthesis of (R)—N—((R)-1-(8-bromo-6-(fluoromethyl)imidazo[1,2-a]pyridin-2-yl)ethyl)-2-methylpropane-2-sulfinamide. To a solution of (R,E)-N-((8-bromo-6-(fluoromethyl)imidazo[1,2-a]pyridin-2-yl)methylene)-2-methylpropane-2-sulfinamide (0.30 g, 0.83 mmol) in THF (10 mL) was added methylmagnesium bromide (0.83 mL, 3 N in ether, 2.5 mmol) at −65° C. and the mixture was stirred at the same temperature for 5 h. The reaction mixture was quenched with NH₄Cl (Sat·aq·, 20 mL) at −65° C. and then extracted with EtOAc (30 mL×3). The combined organics were washed with brine (20 mL), dried over Na₂SO₄, and concentrated in vacuo. The residue was purified by column chromatography on silica gel, eluting with 0˜50% MeOH in DCM to give (R)—N—((R)-1-(8-bromo-6-(fluoromethyl)imidazo[1,2-a]pyridin-2-yl)ethyl)-2-methylpropane-2-sulfinamide (0.20 g, yield: 64%) as pale-yellow solid. ESI-MS [M+H]⁺: 376.1.

Synthesis of (R)—N—((R)-1-(6-(fluoromethyl)-8-(3-methyl-2,4-dioxoimidazolidin-1-yl)imidazo[1,2-a]pyridin-2-yl)ethyl)-2-methylpropane-2-sulfinamide. A mixture of (R)—N—((R)-1-(8-bromo-6-(fluoromethyl)imidazo[1,2-a]pyridin-2-yl)ethyl)-2-methylpropane-2-sulfinamide (101 mg, 0.27 mmol), 3-methylimidazolidine-2,4-dione (160 mg, 1.4 mmol), Pd₂(dba)₃ (38 mg, 0.041 mmol), Xantphos (47 mg, 0.082 mmol) and Cs₂CO₃ (222 mg, 0.68 mmol) in 1,4-dioxane (10 mL) was stirred at 95° C. for 5 h under N₂ atmosphere. The mixture was cooled to room temperature and filtered through Celite®. The filter cake was washed with DCM/MeOH (5/1, 30 mL) and the filtrate was concentrated in vacuo. The residue was purified by column chromatography on silica gel, eluting with 0˜4% MeOH in DCM to give (R)—N—((R)-1-(6-(fluoromethyl)-8-(3-methyl-2,4-dioxoimidazolidin-1-yl)imidazo[1,2-a]pyridin-2-yl)ethyl)-2-methylpropane-2-sulfinamide (100 mg, yield: 90%) as pale-yellow solid. ESI-MS [M+H]+: 410.2

Synthesis of (R)-1-(2-(1-aminoethyl)-6-(fluoromethyl)imidazo[1,2-a]pyridin-8-yl)-3-methylimidazolidine-2,4-dione. A solution of (R)—N—((R)-1-(6-(fluoromethyl)-8-(3-methyl-2,4-dioxoimidazolidin-1-yl)imidazo[1,2-a]pyridin-2-yl)ethyl)-2-methylpropane-2-sulfinamide (100 mg, 0.24 mmol) and HCl (4 N in 1,4-dioxane, 3 mL) in DCM (5 mL) was stirred at room temperature for 1 h. The mixture was concentrated, and the residue was diluted with NH₃ (7 N in MeOH, 5 mL) at 0° C. and stirred at room temperature for 30 min. The resulting mixture was then then concentrated in vacuo, the residue was purified by Prep-TLC (eluting with DCM/MeOH=10:1) to give (R)-1-(2-(1-aminoethyl)-6-(fluoromethyl)imidazo[1,2-a]pyridin-8-yl)-3-methylimidazolidine-2,4-dione (50 mg, yield: 68%) as white solid. ESI-MS [M+H]+: 306.1

Synthesis of (1S,2S)—N-(6-(((R)-1-(6-(fluoromethyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide. A mixture of (R)-1-(2-(1-aminoethyl)-6-(fluoromethyl)imidazo[1,2-a]pyridin-8-yl)-3-methylimidazolidine-2,4-dione (50 mg, 0.16 mmol), DIPEA (0.21 g, 1.6 mmol) and (1S,2S)—N-(6-chloropyrimidin-4-yl)-2-(4-methylpyrimidin-2-yl)cyclopropane-1-carboxamide (72 mg, 0.25 mmol) in iPrOH (2.0 mL) was stirred in sealed tube. After degassing with N₂ for 1 min, the mixture was irradiated in microwave at 140° C. for 2 h. The mixture was cooled to room temperature and concentrated in vacuo. The residue was diluted with H₂O (15 mL) and extracted with EtOAc (30 mL×2). The combined organics were washed with brine (10 mL), dried over Na₂SO₄, and concentrated in vacuo. The residue was purified by Prep-HPLC to give (1S,2S)—N-(6-(((R)-1-(6-(fluoromethyl)-8-(3-methyl-2,4-dioxoimidazolidin-1-yl)imidazo[1,2-a]pyridin-2-yl)ethyl)amino) pyrimidin-4-yl)-2-(4-methylpyrimidin-2-yl)cyclopropane-1-carboxamide (13.8 mg, yield: 15%) as a white solid. ESI-MS [M+H]+: 559.1. Purity: 99.4% (214 nm), 99.5% (254 nm) 1H NMR (400 MHz, DMSO) δ 10.66 (s, 1H), 8.55 (d, J=2.8 Hz, 1H), 8.52 (d, J=5.1 Hz, 1H), 8.16 (s, 1H), 7.83 (s, 2H), 7.66 (s, 1H), 7.32 (s, 1H), 7.21 (d, J=5.1 Hz, 1H), 5.48-5.36 (m, 3H), 5.03-4.94 (m, 2H), 2.98 (s, 3H), 2.64-2.55 (m, 2H), 2.41 (s, 3H), 1.57-1.49 (m, 5H).

### Example 679

### Synthesis of (1S,2S)—N-(6-(((R)-1-(6-isopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (I-679)

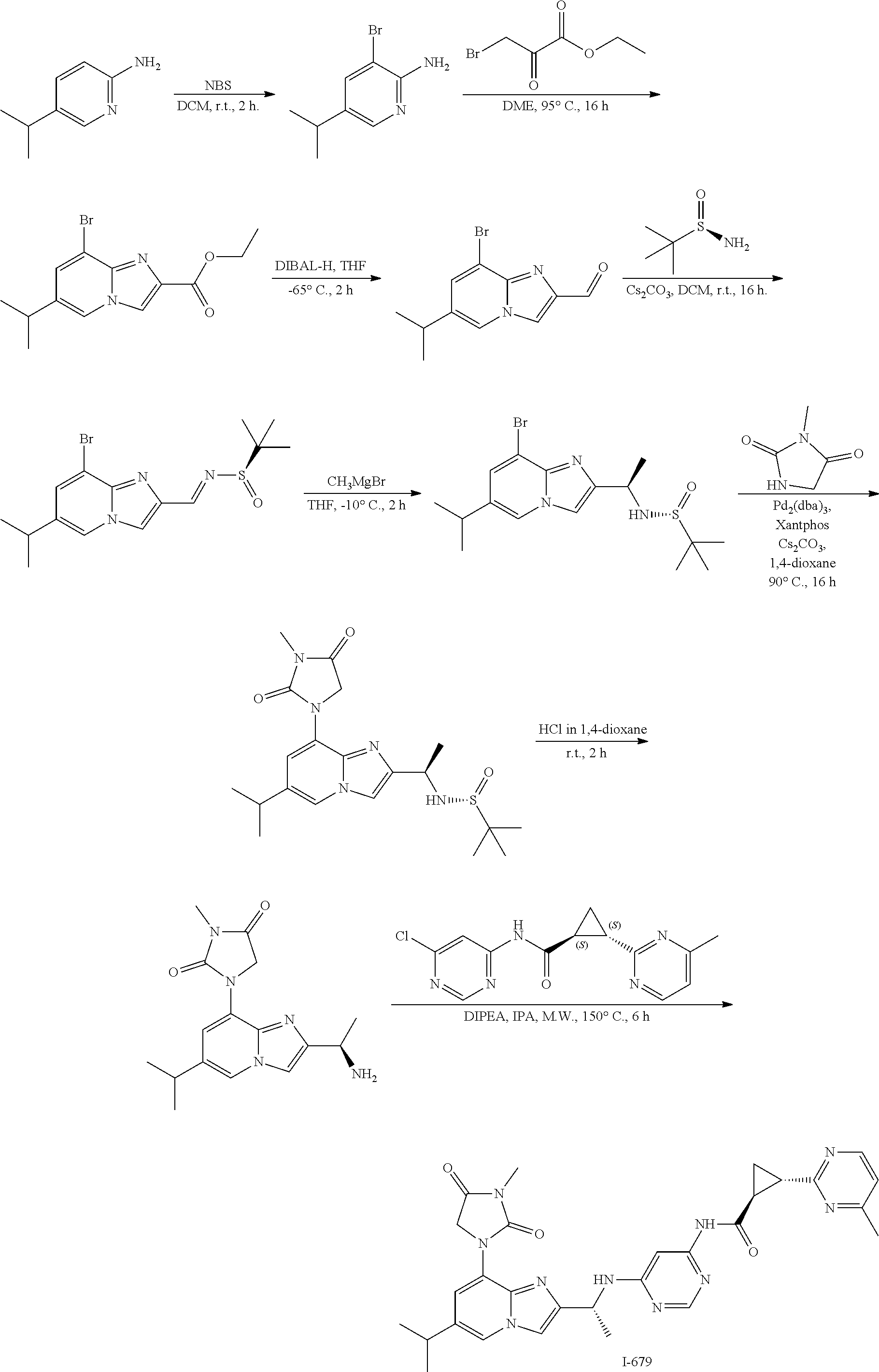

Synthesis of 3-bromo-5-isopropylpyridin-2-amine. A mixture of 5-isopropylpyridin-2-amine (2.0 g, 14.7 mmol) and NBS (3.1 g, 17.6 mmol) in DCM (40 mL) was stirred at room temperature for 2 h. Water (50 mL) was added and the mixture was extracted with EtOAc (30 mL×3). The combined organic layers were washed with brine (60 mL), dried over anhydrous Na₂SO₄, and concentrated in vacuo to give the crude, which was purified by column chromatography (eluent: PE/EtOAc=3/1) to give 3-bromo-5-isopropylpyridin-2-amine (3.0 g, yield: 95%) as a yellow oil. ESI-MS [M+H]+: 215.1.

Synthesis of ethyl 8-bromo-6-isopropylimidazo[1,2-a]pyridine-2-carboxylate. A mixture of 3-bromo-5-isopropylpyridin-2-amine (3.0 g, 14.0 mmol) and ethyl 3-bromo-2-oxopropanoate (8.1 g, 42.0 mmol) in DME (50 mL) was stirred at 95° C. for 16 h. The mixture was cooled to room temperature and concentrated in vacuo. The residue was diluted with NaHCO₃ (sat·aq·, 50 mL) and extracted with DCM (50 mL×3). The combined organic layers were washed with brine (30 mL), dried over Na₂SO₄, and concentrated in vacuo, the residue was purified by column chromatography (eluent: PE/EtOAc=3/1) to give ethyl 8-bromo-6-isopropylimidazo[1,2-a]pyridine-2-carboxylate (2.6 g, yield: 60%) as a brown oil. ESI-MS [M+H]+: 311.2.

Synthesis of 8-bromo-6-isopropylimidazo[1,2-a]pyridine-2-carbaldehyde. To a solution of ethyl 8-bromo-6-isopropylimidazo[1,2-a]pyridine-2-carboxylate (550 mg, 1.8 mmol) in THF (15 mL) was added DIBAL-H (5.4 mL, 1.0 M in hexane, 5.4 mmol) at −65° C. under N₂. After the reaction mixture was stirred at −65° C. for 2 h, the mixture was quenched with NaOH (1M aq·, 10 mL) and extracted with EtOAc (30 mL×3). The combined organic layers were washed with brine (20 mL), dried over anhydrous Na₂SO₄, and concentrated to give the crude, which was purified by column chromatography (eluent: PE/EtOAc=5/1) to give 8-bromo-6-isopropylimidazo[1,2-a]pyridine-2-carbaldehyde (350 mg, yield: 73%) as a yellow solid. ESI-MS [M+H]+: 267.1.

Synthesis of (R,E)-N-((8-bromo-6-isopropylimidazo[1,2-a]pyridin-2-yl)methylene)-2-methylpropane-2-sulfinamide. A mixture of 8-bromo-6-isopropylimidazo[1,2-a]pyridine-2-carbaldehyde (350 mg, 1.32 mmol), (R)-2-methylpropane-2-sulfinamide (240 mg, 1.98 mmol) and Cs₂CO₃ (1.3 g, 3.96 mmol) in DCM (30 mL) was stirred at room temperature for 16 h. The resulting mixture was filtered and the filtrate was concentrated in vacuo. The residue was purified by column chromatography (eluent: PE/EtOAc=3/1) to give (R,E)-N-((8-bromo-6-isopropylimidazo[1,2-a]pyridin-2-yl)methylene)-2-methylpropane-2-sulfinamide (450 mg, yield: 92%) as a yellow solid. ESI-MS [M+H]+: 372.1.

Synthesis of (R)—N—((R)-1-(8-bromo-6-isopropylimidazo[1,2-a]pyridin-2-yl)ethyl)-2-methylpropane-2-sulfinamide. To a solution of (R,E)-N-((8-bromo-6-isopropylimidazo[1,2-a]pyridin-2-yl)methylene)-2-methylpropane-2-sulfinamide (450 mg, 1.2 mmol) in THF (10 mL) was added methylmagnesium bromide (2.0 mL, 3 M in ether, 6.0 mmol) at −10° C. under N₂ and the reaction mixture was stirred at −10° C. for 2 h. The mixture was quenched with NH₄Cl (sat·aq·, 30 mL) and extracted with EtOAc (30 mL×3). The combined organic layers were washed with brine (30 mL), dried over anhydrous Na₂SO₄, and concentrated to give the crude product, which was purified by column chromatography (eluent: DCM/MeOH=20/1) to give (R)—N—((R)-1-(8-bromo-6-isopropylimidazo[1,2-a]pyridin-2-yl)ethyl)-2-methylpropane-2-sulfinamide (300 mg, yield: 65%) as a yellow solid. ESI-MS [M+H]+: 388.1.

Synthesis of (R)—N—((R)-1-(6-isopropyl-8-(3-methyl-2,4-dioxoimidazolidin-1-yl)imidazo[1,2-a]pyridin-2-yl)ethyl)-2-methylpropane-2-sulfinamide. To a mixture of (R)—N—((R)-1-(8-bromo-6-isopropylimidazo[1,2-a]pyridin-2-yl)ethyl)-2-methylpropane-2-sulfinamide (300 mg, 0.78 mmol), 3-methylimidazolidine-2,4-dione (133 mg, 1.17 mmol) and Cs₂CO₃ (763 mg, 2.34 mmol) in 1,4-dioxane (15 mL) was added Pd₂(dba)₃ (71 mg, 0.078 mmol) and Xantphos (90 mg, 0.16 mmol). The mixture was stirred at 90° C. for 16 h under N₂. The reaction mixture was cooled to room temperature and filtered through Celite®, the filter cake was washed with DCM/MeOH (10/1, 30 mL). The filtrate was concentrated in vacuo, the residue was purified by column chromatography (eluent: DCM/MeOH=20/1) to give (R)—N—((R)-1-(6-isopropyl-8-(3-methyl-2,4-dioxoimidazolidin-1-yl)imidazo[1,2-a]pyridin-2-yl)ethyl)-2-methylpropane-2-sulfinamide (80 mg, yield: 24%) as a yellow solid. ESI-MS [M+H]+: 420.2.

Synthesis of (R)-1-(2-(1-aminoethyl)-6-isopropylimidazo[1,2-a]pyridin-8-yl)-3-methylimidazolidine-2,4-dione. To a solution of (R)—N—((R)-1-(6-isopropyl-8-(3-methyl-2,4-dioxoimidazolidin-1-yl)imidazo[1,2-a]pyridin-2-yl)ethyl)-2-methylpropane-2-sulfinamide (80 mg, 0.19 mmol) in DCM (5 mL) was added HCl (4M solution in 1,4-dioxane, 2 mL) and the reaction mixture was stirred at room temperature for 2 h. The mixture was concentrated, and the residue was diluted with NH₃ (7M solution in MeOH, 5 mL). The resulting mixture was concentrated in vacuo, the residue was purified by Prep-TLC (eluent: DCM/MeOH=10/1) to give (R)-1-(2-(1-aminoethyl)-6-isopropylimidazo[1,2-a]pyridin-8-yl)-3-methylimidazolidine-2,4-dione (50 mg, yield: 83%) as a yellow solid. ESI-MS [M+H]+: 316.2.

Synthesis of (1S,2S)—N-(6-(((R)-1-(6-isopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide. A mixture of (R)-1-(2-(1-aminoethyl)-6-isopropylimidazo[1,2-a]pyridin-8-yl)-3-methylimidazolidine-2,4-dione (50 mg, 0.16 mmol), (1S,2S)—N-(6-chloropyrimidin-4-yl)-2-(4-methylpyrimidin-2-yl)cyclopropane-1-carboxamide (69 mg, 0.24 mmol) and DIPEA (103 mg, 0.80 mmol) in i-PrOH (5 mL) was stirred in a sealed tube. After degassing with N₂ for 1 min, the reaction was irradiated in microwave at 150° C. for 6 h. The mixture was cooled to room temperature and concentrated to give the crude, which was purified by Prep-TLC (eluent: DCM/MeOH=20/1) to give (1S,2S)—N-(6-(((R)-1-(6-isopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide (36 mg, yield: 40%) as a white solid. ESI-MS [M+H]+: 569.2. Purity: 99.16 (214 nm), 99.18 (254 nm). 1H NMR (400 MHz, DMSO) δ 10.65 (s, 1H), 8.52 (d, J=5.1 Hz, 1H), 8.25 (s, 1H), 8.15 (d, J=0.8 Hz, 1H), 7.78 (s, 1H), 7.69 (s, 1H), 7.52 (d, J=1.4 Hz, 1H), 7.30 (s, 1H), 7.21 (d, J=5.1 Hz, 1H), 5.32 (s, 1H), 4.95 (d, J=1.5 Hz, 1H), 2.98 (s, 3H), 2.92-2.85 (m, 1H), 2.60-2.52 (m, 2H), 2.41 (s, 3H), 1.52-1.50 (m, 5H), 1.22 (d, J=6.9 Hz, 6H).

### Example 680

Inhibitory Activity of Exemplary Compounds against Plasma Kallikrein.

Compounds were evaluated for inhibition of the human activated kallikrein enzyme in two formats of an assay employing a fluorogenic peptide substrate. In one assay format, the concentrations of reagents were as follows: 20 mM Tris pH 7.5, 1 mM EDTA, 150 mM sodium chloride, 0.1% PEG-400, 0.1% Triton X-100, 500 pM activated kallikrein enzyme, 300 uM Pro-Phe-Arg-7-amido-4-methylcoumarin (PFR-AMC) substrate. Prior to reaction initiation with substrate, enzyme and inhibitors were preincubated for 30 min at room temperature. After initiation with substrate, reactions were incubated for 10 min at room temperature and fluorescence emission at 460 nm from 380 nm excitation measured with a microplate reader. In another assay format, the concentrations of reagents were as follows: 20 mM Tris pH 7.5, 1 mM EDTA, 150 mM sodium chloride, 0.1% PEG-400, 0.1% Triton X-100, 5 pM activated kallikrein enzyme, 300 uM PFR-AMC substrate. Prior to reaction initiation with substrate, enzyme and inhibitors were preincubated for 30 min at room temperature. After initiation with substrate, reactions were incubated for 18 hr at room temperature and fluorescence emission at 460 nm from 380 nm excitation measured with a microplate reader.

Table 49 provides the results of the assay in the format with 500 pM activated kallikrein assay. For the compounds listed in Table 49, the EC₅₀ values are reported according to the following ranges: A≤1.0 nM; 1.0 nM<B≤10 nM; 10 nM<C≤100 nM; 100 nM<D≤1000 nM; 1000 nM<E≤6000 nM; 6000 nM<F.

TABLE 49

| | Primary |
|---|---|
| | Dose |
| | Response |
| Compound | Assay: |
| I-# | Average EC₅₀ |
| 1 | B |
| 2 | A |
| 3 | B |
| 4 | A |
| 5 | A |
| 6 | B |
| 7 | B |
| 8 | B |
| 9 | B |
| 10 | B |
| 11 | B |
| 12 | B |
| 13 | B |
| 14 | B |
| 15 | B |
| 16 | A |
| 17 | A |
| 18 | A |
| 19 | A |
| 20 | B |
| 21 | B |
| 22 | C |
| 23 | A |
| 24 | A |
| 25 | A |
| 26 | A |
| 27 | A |
| 28 | A |
| 29 | D |
| 30 | A |
| 31 | B |
| 32 | A |
| 33 | A |
| 34 | A |
| 35 | A |
| 36 | A |
| 37 | A |
| 38 | B |
| 39 | B |
| 40 | A |
| 41 | C |
| 42 | A |
| 43 | C |
| 44 | C |
| 45 | D |
| 46 | C |
| 47 | D |
| 48 | B |
| 49 | D |
| 50 | B |
| 51 | C |
| 52 | D |
| 53 | D |
| 54 | C |
| 55 | B |
| 56 | D |
| 57 | B |
| 58 | B |
| 59 | B |
| 60 | B |
| 61 | B |
| 62 | B |
| 63 | B |
| 64 | B |
| 65 | B |
| 66 | B |
| 67 | B |
| 68 | B |
| 69 | E |
| 70 | B |
| 71 | B |
| 72 | B |
| 73 | B |
| 74 | A |
| 75 | A |
| 76 | D |
| 77 | C |
| 78 | D |
| 79 | A |
| 80 | A |
| 81 | C |
| 82 | A |
| 83 | C |
| 84 | A |
| 85 | A |
| 86 | D |
| 87 | B |
| 88 | A |
| 89 | A |
| 90 | A |
| 91 | A |
| 92 | A |
| 93 | B |
| 94 | A |
| 95 | A |
| 96 | A |
| 97 | A |
| 98 | A |
| 99 | A |
| 100 | A |
| 101 | B |
| 102 | B |
| 103 | C |
| 104 | D |
| 105 | D |
| 106 | D |
| 107 | C |
| 108 | B |
| 109 | B |
| 110 | C |
| 111 | B |
| 112 | B |
| 113 | A |
| 114 | B |
| 115 | B |
| 116 | D |
| 117 | A |
| 118 | A |
| 119 | B |
| 120 | A |
| 121 | A |
| 122 | B |
| 123 | A |
| 124 | B |
| 125 | B |
| 126 | A |
| 127 | A |
| 128 | A |
| 129 | A |
| 130 | A |
| 131 | A |
| 132 | A |
| 133 | A |
| 134 | A |
| 135 | A |
| 137 | A |
| 138 | A |
| 139 | A |
| 140 | A |
| 141 | D |
| 142 | A |
| 143 | A |
| 144 | A |
| 145 | C |
| 146 | B |
| 147 | B |
| 148 | C |
| 149 | B |
| 150 | C |
| 151 | B |
| 152 | C |
| 153 | B |
| 154 | B |
| 155 | A |
| 156 | D |
| 157 | A |
| 158 | D |
| 159 | B |
| 160 | C |
| 161 | A |
| 162 | B |
| 163 | A |
| 164 | B |
| 165 | B |
| 166 | A |
| 167 | A |
| 168 | A |
| 169 | A |
| 170 | A |
| 171 | B |
| 172 | A |
| 173 | A |
| 174 | A |
| 175 | F |
| 176 | D |
| 177 | A |
| 178 | D |
| 179 | D |
| 180 | C |
| 181 | F |
| 182 | F |
| 183 | C |
| 184 | C |
| 185 | D |
| 186 | C |
| 187 | F |
| 188 | F |
| 189 | F |
| 190 | F |
| 191 | F |
| 192 | A |
| 193 | A |
| 194 | A |
| 195 | A |
| 196 | B |
| 197 | C |
| 198 | A |
| 199 | B |
| 200 | A |
| 201 | B |
| 202 | B |
| 203 | C |
| 204 | D |
| 205 | C |
| 206 | C |
| 207 | B |
| 208 | A |
| 209 | A |
| 210 | D |
| 211 | C |
| 212 | B |
| 213 | B |
| 214 | B |
| 216 | F |
| 217 | B |
| 218 | C |
| 219 | B |
| 220 | C |
| 221 | C |
| 222 | B |
| 223 | A |
| 224 | A |
| 225 | F |
| 226 | C |
| 227 | A |
| 228 | A |
| 229 | A |
| 230 | B |
| 231 | A |
| 232 | A |
| 233 | A |
| 234 | A |
| 235 | A |
| 236 | A |
| 237 | A |
| 238 | A |
| 239 | A |
| 240 | B |
| 241 | A |
| 242 | A |
| 243 | A |
| 244 | A |
| 245 | A |
| 246 | A |
| 247 | B |
| 248 | E |
| 249 | B |
| 250 | F |
| 251 | C |
| 252 | A |
| 253 | B |
| 254 | A |
| 255 | B |
| 256 | C |
| 257 | B |
| 258 | E |
| 259 | A |
| 260 | A |
| 261 | C |
| 262 | A |
| 263 | C |
| 264 | A |
| 265 | B |
| 266 | D |
| 267 | B |
| 268 | A |
| 269 | C |
| 270 | A |
| 271 | A |
| 272 | E |
| 273 | A |
| 274 | B |
| 275 | B |
| 276 | A |
| 277 | D |
| 278 | D |
| 279 | D |
| 280 | F |
| 281 | B |
| 282 | B |
| 283 | B |
| 284 | A |
| 285 | A |
| 286 | C |
| 287 | A |
| 288 | A |
| 289 | A |
| 290 | A |
| 291 | A |
| 292 | A |
| 293 | A |
| 294 | A |
| 295 | A |
| 296 | A |
| 297 | E |
| 298 | B |
| 299 | A |
| 300 | A |
| 301 | A |
| 302 | B |
| 303 | B |
| 304 | A |
| 305 | B |
| 306 | A |
| 307 | A |
| 308 | B |
| 309 | A |
| 310 | D |
| 311 | C |
| 312 | C |
| 313 | B |
| 314 | C |
| 315 | D |
| 316 | B |
| 317 | A |
| 318 | A |
| 319 | A |
| 320 | D |
| 321 | D |
| 322 | A |
| 323 | C |
| 324 | A |
| 325 | A |
| 326 | A |
| 327 | A |
| 328 | C |
| 329 | B |
| 330 | D |
| 331 | B |
| 332 | D |
| 333 | A |
| 334 | A |
| 335 | D |
| 336 | A |
| 337 | B |
| 338 | A |
| 339 | B |
| 340 | B |
| 341 | B |
| 342 | A |
| 343 | A |
| 344 | B |
| 345 | C |
| 346 | B |
| 347 | C |
| 348 | B |
| 349 | F |
| 350 | F |
| 351 | F |
| 352 | C |
| 353 | A |
| 354 | A |
| 355 | A |
| 356 | A |
| 357 | A |
| 358 | A |
| 359 | A |
| 360 | B |
| 361 | A |
| 362 | D |
| 363 | A |
| 364 | A |
| 365 | A |
| 366 | A |
| 367 | A |
| 368 | A |
| 369 | B |
| 370 | C |
| 371 | B |
| 372 | A |
| 373 | B |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | B |
| 387 | A |
| 388 | A |
| 389 | A |
| 390 | A |
| 391 | A |
| 392 | A |
| 393 | A |
| 394 | A |
| 395 | A |
| 396 | A |
| 397 | C |
| 398 | B |
| 399 | B |
| 400 | B |
| 401 | A |
| 402 | A |
| 403 | A |
| 404 | A |
| 405 | A |
| 406 | A |
| 407 | A |
| 408 | B |
| 409 | C |
| 410 | A |
| 411 | A |
| 412 | B |
| 413 | D |
| 414 | A |
| 415 | A |
| 416 | C |
| 417 | A |
| 418 | A |
| 419 | A |
| 420 | A |
| 421 | A |
| 422 | A |
| 423 | A |
| 424 | A |
| 425 | C |
| 426 | C |
| 427 | C |
| 428 | A |
| 429 | A |
| 430 | B |
| 431 | A |
| 432 | A |
| 433 | A |
| 434 | A |
| 435 | A |
| 436 | A |
| 437 | A |
| 438 | A |
| 439 | A |
| 440 | A |
| 441 | A |
| 442 | E |
| 443 | A |
| 444 | A |
| 445 | A |
| 446 | A |
| 447 | A |
| 448 | D |
| 449 | A |
| 450 | D |
| 451 | A |
| 452 | C |
| 453 | F |
| 454 | A |
| 455 | C |
| 456 | A |
| 457 | B |
| 458 | B |
| 459 | B |
| 460 | B |
| 461 | B |
| 462 | A |
| 463 | A |
| 464 | A |
| 465 | A |
| 466 | A |
| 467 | D |
| 468 | A |
| 469 | A |
| 470 | B |
| 471 | A |
| 472 | B |
| 473 | A |
| 474 | A |
| 475 | A |
| 476 | A |
| 477 | B |
| 478 | A |
| 479 | B |
| 480 | B |
| 481 | A |
| 482 | A |
| 483 | D |
| 484 | F |
| 485 | E |
| 486 | B |
| 487 | A |
| 488 | A |
| 489 | A |
| 490 | A |
| 491 | A |
| 492 | A |
| 493 | B |
| 494 | A |
| 495 | B |
| 496 | A |
| 497 | A |
| 498 | A |
| 499 | B |
| 500 | A |
| 501 | A |
| 502 | A |
| 503 | B |
| 504 | C |
| 505 | C |
| 506 | F |
| 507 | F |
| 508 | F |
| 509 | A |
| 510 | D |
| 511 | B |
| 512 | B |
| 513 | A |
| 514 | C |
| 515 | A |
| 516 | A |
| 517 | A |
| 518 | A |
| 519 | A |
| 520 | A |
| 521 | A |
| 522 | A |
| 523 | A |
| 524 | A |
| 525 | A |
| 526 | A |
| 527 | B |
| 528 | B |
| 529 | A |
| 530 | A |
| 531 | A |
| 532 | A |
| 533 | A |
| 534 | A |
| 535 | A |
| 536 | A |
| 537 | A |
| 538 | A |
| 539 | A |
| 540 | A |
| 541 | A |
| 542 | C |
| 543 | B |
| 544 | D |
| 545 | A |
| 546 | A |
| 547 | A |
| 548 | D |
| 549 | A |
| 550 | A |
| 551 | A |
| 552 | A |
| 553 | A |
| 554 | D |
| 555 | A |
| 556 | A |
| 557 | A |
| 558 | A |
| 559 | B |
| 560 | A |
| 561 | A |
| 562 | A |
| 563 | A |
| 564 | A |
| 565 | B |
| 566 | B |
| 567 | C |
| 568 | A |
| 569 | D |
| 570 | C |
| 571 | A |
| 572 | B |
| 573 | A |
| 574 | B |
| 575 | A |
| 576 | A |
| 577 | A |
| 578 | B |
| 579 | A |
| 580 | B |
| 581 | B |
| 582 | A |
| 583 | B |
| 584 | E |
| 585 | C |
| 586 | A |
| 587 | D |
| 588 | C |
| 589 | C |
| 590 | C |
| 591 | B |
| 592 | B |
| 593 | F |
| 594 | C |
| 595 | A |
| 596 | C |
| 597 | B |
| 598 | A |
| 599 | A |
| 600 | A |
| 601 | A |
| 602 | A |
| 603 | A |
| 604 | B |
| 605 | A |
| 606 | F |
| 607 | A |
| 608 | A |
| 609 | A |
| 610 | B |
| 611 | A |
| 612 | A |
| 613 | B |
| 614 | B |
| 615 | E |
| 616 | F |
| 617 | A |
| 618 | A |
| 619 | E |
| 620 | B |
| 621 | A |
| 622 | A |
| 623 | A |
| 624 | A |
| 625 | A |
| 626 | A |
| 627 | A |
| 628 | A |
| 629 | A |
| 630 | B |
| 631 | A |
| 632 | A |
| 633 | D |
| 634 | A |
| 635 | A |
| 636 | B |
| 637 | A |
| 638 | A |
| 639 | A |
| 640 | A |
| 641 | A |
| 642 | A |
| 643 | A |
| 644 | A |
| 645 | C |
| 646 | F |
| 647 | A |
| 648 | A |
| 649 | A |
| 650 | A |
| 651 | A |
| 652 | A |
| 653 | A |
| 654 | A |
| 655 | A |
| 656 | A |
| 657 | A |
| 658 | A |
| 659 | B |
| 660 | C |
| 661 | B |
| 662 | A |
| 663 | A |
| 664 | A |
| 665 | A |
| 666 | A |
| 667 | A |
| 668 | A |
| 669 | A |
| 670 | A |
| 671 | A |
| 672 | A |
| 673 | D |
| 674 | C |
| 675 | A |
| 676 | A |
| 677 | A |
| 678 | A |
| 679 | A |

### Example 681

### Neat Human and Rat Plasma Assay

To analyze inhibition of plasma kallikrein in an ex vivo setting, the potency of compounds was measure in contact pathway-activated plasma assays. In a fluorogenic peptide substrate assay, test compounds dissolved in DMSO were added to sodium citrate collected human or rat plasma in a 96-well microplate. Alternatively, citrated plasma was collected from rats administered the compounds orally or by IV. 10 nM of human FXIIa (Enzyme Research Laboratories) diluted in pKal buffer (20 mM Tris-HCl, pH 7.5, 150 mM NaCl, 1 mM EDTA, 0.1% PEG-8000, and 0.1% Triton X-100) was added to the plasma, followed by the 100 μM of the profluorescent, synthetic plasma kallikrein substrate PFR-AMC (also diluted in pKal buffer). Final plasma concentration in the reaction was 78%. Fluorescence was immediately monitored by excitation/emission wavelengths of 360 nm/480 nm respectively over a period of 5 minutes in a microplate reader. The resulting linear increase in fluorescence emission (reflecting pKal proteolysis of PFR-AMC substrate) was fit to extract a proteolytic rate (fluorescent units over time), and this rate was subsequently plotted against compound inhibitor concentration. Resulting plots were fit to a standard 4-parameter IC50/IC90 equation to determine min/max values, IC50/90, and slope. All experimental steps were performed at room temperature. Table 50 provides results of the assay.

For the compounds listed in Table 50, the IC₉₀ values are reported according to the following ranges: A≤400 nM; 400 nM<B≤1500 nM; 1500 nM<C≤10000 nM; 10000 nM<D≤48000 nM; 48000 nM<E.

TABLE 50

| | Human |
|---|---|
| Compound | Plasma Assay: |
| I-# | IC90 |
| 2 | B |
| 4 | C |
| 5 | B |
| 6 | C |
| 7 | B |
| 9 | C |
| 10 | C |
| 11 | C |
| 13 | C |
| 16 | B |
| 17 | B |
| 18 | A |
| 19 | A |
| 21 | C |
| 23 | B |
| 24 | C |
| 25 | B |
| 26 | C |
| 27 | A |
| 28 | B |
| 30 | A |
| 31 | D |
| 32 | A |
| 33 | B |
| 34 | A |
| 40 | B |
| 49 | A |
| 50 | D |
| 57 | E |
| 60 | D |
| 62 | C |
| 63 | C |
| 64 | E |
| 70 | E |
| 71 | D |
| 79 | B |
| 80 | A |
| 82 | D |
| 85 | B |
| 87 | D |
| 88 | B |
| 90 | B |
| 91 | B |
| 92 | C |
| 95 | C |
| 96 | B |
| 97 | B |
| 99 | B |
| 100 | D |
| 101 | D |
| 108 | D |
| 113 | B |
| 114 | B |
| 117 | C |
| 120 | D |
| 122 | D |
| 123 | D |
| 125 | D |
| 126 | D |
| 127 | A |
| 128 | B |
| 129 | C |
| 130 | B |
| 131 | D |
| 132 | B |
| 133 | B |
| 134 | A |
| 135 | A |
| 137 | C |
| 138 | A |
| 139 | B |
| 140 | B |
| 142 | C |
| 143 | B |
| 144 | B |
| 147 | C |
| 151 | C |
| 153 | C |
| 155 | C |
| 157 | C |
| 159 | D |
| 161 | C |
| 162 | C |
| 163 | B |
| 164 | B |
| 166 | B |
| 167 | B |
| 168 | B |
| 169 | B |
| 170 | C |
| 172 | B |
| 173 | B |
| 174 | C |
| 192 | C |
| 193 | A |
| 194 | C |
| 195 | B |
| 196 | C |
| 198 | A |
| 199 | C |
| 200 | B |
| 202 | B |
| 207 | C |
| 208 | A |
| 209 | B |
| 217 | D |
| 219 | D |
| 222 | E |
| 223 | E |
| 224 | D |
| 227 | A |
| 228 | B |
| 229 | A |
| 231 | B |
| 232 | C |
| 233 | B |
| 234 | A |
| 235 | B |
| 236 | E |
| 237 | C |
| 238 | A |
| 239 | C |
| 240 | C |
| 242 | C |
| 243 | B |
| 244 | A |
| 249 | A |
| 254 | A |
| 259 | A |
| 260 | A |
| 262 | B |
| 264 | B |
| 267 | D |
| 268 | D |
| 270 | C |
| 274 | C |
| 275 | C |
| 276 | A |
| 281 | D |
| 282 | C |
| 283 | C |
| 284 | A |
| 285 | A |
| 287 | D |
| 288 | C |
| 289 | C |
| 290 | C |
| 291 | C |
| 292 | C |
| 293 | C |
| 294 | D |
| 295 | C |
| 296 | C |
| 299 | B |
| 300 | C |
| 301 | C |
| 304 | A |
| 305 | C |
| 307 | A |
| 309 | C |
| 317 | C |
| 318 | B |
| 319 | B |
| 322 | B |
| 324 | B |
| 326 | B |
| 327 | A |
| 333 | B |
| 337 | D |
| 338 | C |
| 341 | C |
| 342 | C |
| 343 | C |
| 344 | C |
| 353 | B |
| 354 | A |
| 355 | A |
| 356 | C |
| 357 | A |
| 358 | B |
| 359 | B |
| 360 | B |
| 361 | B |
| 363 | C |
| 364 | B |
| 365 | A |
| 366 | A |
| 367 | B |
| 368 | A |
| 369 | C |
| 371 | C |
| 372 | A |
| 373 | B |
| 374 | A |
| 375 | A |
| 376 | A |
| 377 | B |
| 378 | A |
| 379 | B |
| 381 | D |
| 382 | A |
| 383 | A |
| 384 | A |
| 385 | A |
| 386 | C |
| 387 | A |
| 388 | B |
| 389 | B |
| 390 | A |
| 391 | A |
| 392 | B |
| 393 | B |
| 394 | B |
| 395 | A |
| 396 | B |
| 399 | C |
| 400 | C |
| 401 | B |
| 402 | B |
| 403 | B |
| 404 | B |
| 406 | A |
| 407 | B |
| 408 | B |
| 410 | A |
| 411 | A |
| 412 | B |
| 414 | D |
| 415 | A |
| 418 | A |
| 423 | A |
| 424 | B |
| 434 | A |
| 437 | A |
| 443 | A |
| 446 | B |
| 447 | A |
| 449 | C |
| 451 | B |
| 454 | B |
| 456 | A |
| 457 | B |
| 458 | C |
| 459 | C |
| 460 | C |
| 462 | A |
| 463 | B |
| 464 | A |
| 465 | B |
| 466 | C |
| 470 | C |
| 471 | B |
| 472 | C |
| 473 | B |
| 474 | B |
| 475 | A |
| 476 | C |
| 477 | C |
| 478 | B |
| 479 | C |
| 480 | A |
| 481 | C |
| 482 | B |
| 487 | A |
| 488 | B |
| 489 | B |
| 490 | B |
| 491 | B |
| 492 | B |
| 493 | B |
| 494 | A |
| 495 | B |
| 496 | A |
| 497 | B |
| 498 | B |
| 499 | B |
| 500 | B |
| 501 | B |
| 502 | B |
| 512 | C |
| 513 | C |
| 515 | B |
| 516 | A |
| 517 | A |
| 518 | A |
| 519 | A |
| 520 | A |
| 521 | B |
| 522 | B |
| 523 | A |
| 524 | B |
| 525 | A |
| 526 | B |
| 529 | B |
| 534 | A |
| 539 | C |
| 540 | A |
| 541 | B |
| 543 | C |
| 545 | C |
| 546 | B |
| 549 | B |
| 550 | B |
| 551 | B |
| 552 | C |
| 553 | A |
| 555 | B |
| 556 | B |
| 557 | B |
| 558 | A |
| 559 | B |
| 560 | A |
| 561 | A |
| 562 | B |
| 563 | A |
| 564 | A |
| 565 | C |
| 566 | D |
| 568 | C |
| 573 | B |
| 574 | D |
| 575 | A |
| 595 | A |
| 598 | A |
| 599 | A |
| 600 | E |
| 601 | B |
| 602 | A |
| 603 | A |
| 604 | D |
| 605 | B |
| 607 | A |
| 608 | A |
| 609 | A |
| 610 | D |
| 611 | C |
| 612 | C |
| 613 | B |
| 614 | D |
| 617 | A |
| 618 | B |
| 620 | B |
| 621 | A |
| 622 | B |
| 623 | A |
| 625 | B |
| 627 | A |
| 628 | B |
| 636 | C |
| 637 | A |
| 638 | B |
| 639 | A |
| 640 | A |
| 641 | B |
| 647 | B |
| 678 | A |
| 679 | A |

### Example 683

### Comparative Inhibitory Activity of Various Compounds Against Plasma Kallikrein.

Table 51 provides comparative plasma kallikrein inhibition potency in the assay format with 500 pM activated kallikrein assay (described in detail in a preceding example). For the compounds listed in Table 51, the EC₅₀ values are reported according to the following ranges: A≤1.0 nM; 1.0 nM<B≤10 nM; 10 nM<C≤100 nM; 100 nM<D≤1000 nM; 1000 nM<E≤6000 nM; 6000 nM<F.

TABLE 51

| | Primary Dose Response Assay: |
|---|---|
| Compound | Average EC50 |
| 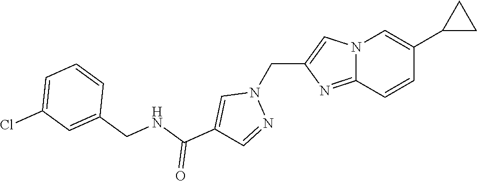 [SMILES: O=C(NCc1cccc(Cl)c1)c1cnn(Cc2cn3cc(C4CC4)ccc3n2)c1] | E |
| 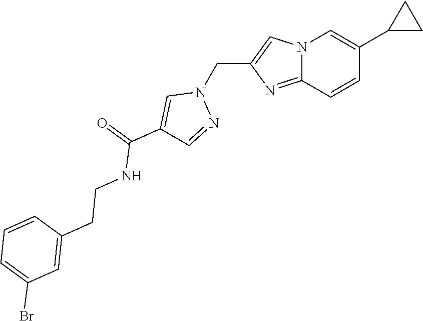 [SMILES: O=C(NCCc1cccc(Br)c1)c1cnn(Cc2cn3cc(C4CC4)ccc3n2)c1] | E |
| 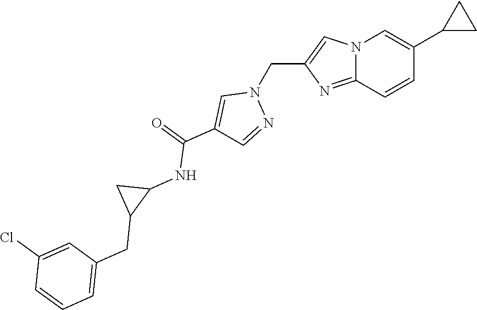 [SMILES: O=C(NC1CC1Cc1cccc(Cl)c1)c1cnn(Cc2cn3cc(C4CC4)ccc3n2)c1] | E |
| 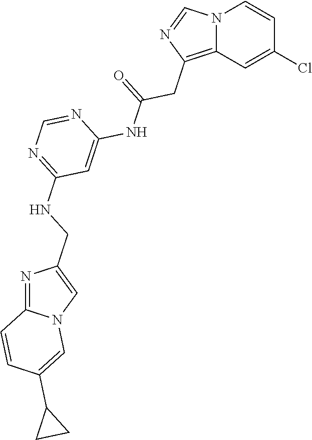 [SMILES: O=C(Cc1ncn2ccc(Cl)cc12)Nc1cc(NCc2cn3cc(C4CC4)ccc3n2)ncn1] | B |
| 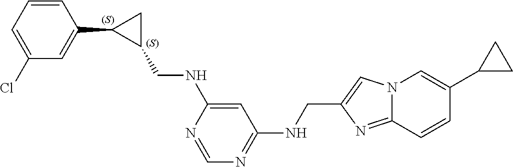 | D |
| 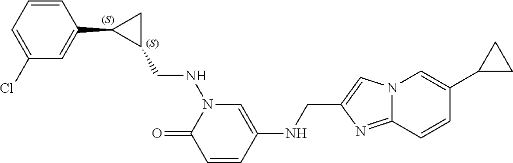 | E |
| 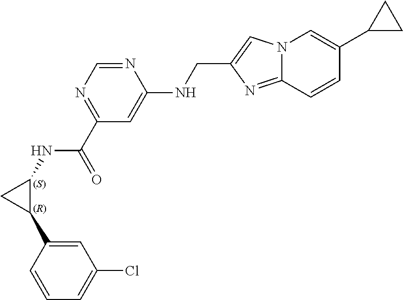 | E |
| 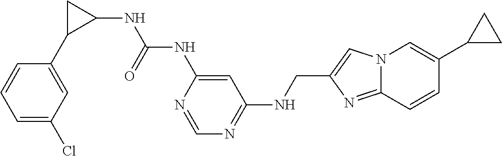 [SMILES: O=C(Nc1cc(NCc2cn3cc(C4CC4)ccc3n2)ncn1)NC1CC1c1cccc(Cl)c1] | E |

### EXEMPLARY ENUMERATED EMBODIMENTS

1. A compound of Formula (I):

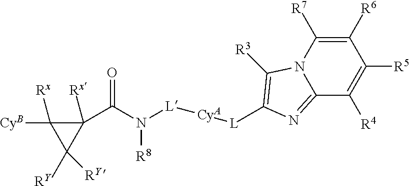

or a pharmaceutically acceptable salt thereof,
wherein:
Cy^{A} is a phenylene or a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 —R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}—, —S—, —SO—, or SO₂—;
each R³, R⁴, R⁵, R⁶, and R⁷ is independently selected from hydrogen or -L^{C}-R^{C}, wherein
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
      each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4-L^{D}-R^{D} groups;
         each L^{D} is independently selected from a covalent bond or an optionally substituted C₁₋₆ hydrocarbon chain, wherein 1 to 3 methylene units are optionally and independently replaced with —O— or —NR—;
         each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and
R⁸ is selected from hydrogen, —OR, or an optionally substituted C₁₋₆ aliphatic group.

2. The compound of embodiment 1, wherein Cy^{A} is a phenylene, wherein Cy^{A} is substituted with 0-4 —R^{A} groups.

3. The compound of any one of the preceding embodiments, wherein Cy^{A} is a 5- to 6-membered monocyclic heteroarylene having 1-3 heteroatoms independently selected from oxygen, nitrogen, and sulfur, wherein Cy^{A} is substituted with 0-4 —R^{A} groups.

4. The compound of embodiment 3, wherein Cy^{A} is a 6-membered monocyclic heteroarylene having 1-3 heteroatoms independently selected from oxygen, nitrogen, and sulfur, wherein Cy^{A} is substituted with 0-4 —R^{A} groups.

5. The compound of embodiments 3 or 4, wherein Cy^{A} is pyridinediyl, pyrimidinediyl, pyridazinediyl, or triazinediyl substituted with 0-1 R^{A} groups.

6. The compound of embodiment 3, wherein Cy^{A} is a 5-membered monocyclic heteroarylene having 1-3 heteroatoms independently selected from oxygen, nitrogen, and sulfur, wherein Cy^{A} is substituted with 0-2 —R^{A} groups.

7. The compound of embodiment 6, wherein Cy^{A} is unsubstituted thiadiazolediyl, unsubstituted oxadiazolediyl, or unsubstituted triazolediyl.

8. The compound of embodiment 1, wherein Cy^{A} is a 7- to 12-membered bicyclic heteroarylene having 1-4 heteroatoms independently selected from oxygen, nitrogen, and sulfur, wherein Cy^{A} is substituted with 0-4 —R^{A} groups.

9. The compound of embodiment 8, wherein Cy^{A} is a 9-membered bicyclic heteroarylene having 3-4 heteroatoms independently selected from oxygen and nitrogen, or a 10-membered bicyclic heteroarylene having 3-4 heteroatoms independently selected from oxygen and nitrogen, wherein CyA is substituted with 0-1 —R^{A} groups.

10. The compound of embodiment 4, wherein Cy^{A} is selected from the group consisting of:

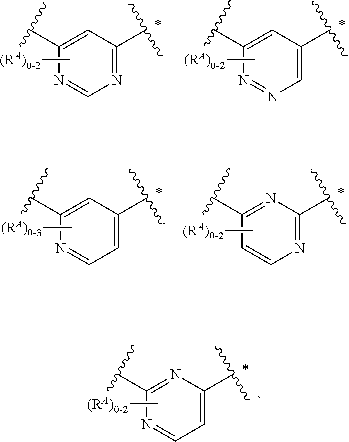

wherein * represents the point of attachment to L.

11. The compound of embodiment 10, wherein Cy^{A} is

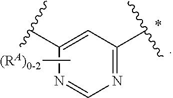

12. The compound of embodiment 10, wherein Cy^{A} is

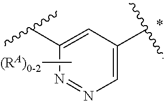

13. The compound of embodiment 10, wherein Cy^{A} is

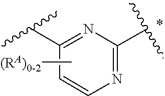

14. The compound of embodiment 6, wherein Cy^{A} is selected from the group consisting of:

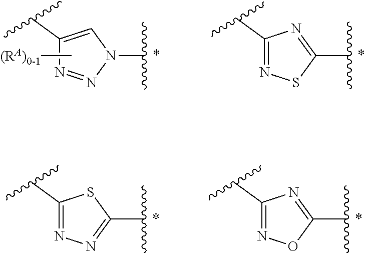

wherein * represents the point of attachment to L.

15. The compound of any one of the preceding embodiments, wherein each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur.

16. The compound of any one of the preceding embodiments, wherein Cy^{A} is substituents on an optionally substituted R^{A} group are independently halogen, —(CH₂)₀₋₄OR^{∘}, or —(CH₂)₀₋₄N(R^{∘})₂, wherein each R^{∘} is independently as defined above and described in classes and subclasses herein.

17. The compound of any one of the preceding embodiments, wherein Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 —R^{B} groups.

18. The compound of any one of the preceding embodiments, wherein Cy^{B} is phenyl or a 5- to 6-membered heteroaryl having 1-3 heteroatoms independently selected from oxygen, nitrogen, and sulfur, wherein Cy^{B} is substituted with 0-4 —R^{B} groups.

19. The compound of any one of the preceding embodiments, wherein Cy^{B} is phenyl, wherein Cy^{B} is substituted with 0-4 —R^{B} groups.

20. The compound of any one of embodiments 1-18, wherein Cy^{B} is a 6-membered heteroaryl having 1-3 nitrogens, wherein Cy^{B} is substituted with 0-4 —R^{B} groups.

21. The compound of embodiment 20, wherein Cy^{B} a pyrimidinyl group substituted with 0-2 —R^{B} groups, a pyridinyl group substituted with 0-2 —R^{B} groups, a pyrazinyl group substituted with 0-1 —R^{B} groups, a pyridazinyl group substituted with 0-1 —R^{B} groups, or a 1,3,5-triazinyl group substituted with 0-1 —R^{B} groups.

22. The compound of any one of embodiments 1-18, wherein Cy^{B} is a 5-membered heteroaryl having 1-2 heteroatoms independently selected from oxygen, nitrogen, and sulfur, wherein Cy^{B} is substituted with 0-4 —R^{B} groups.

23. The compound of embodiment 22, wherein Cy^{B} is a thienyl group substituted with 0-2 —R^{B} groups, a thiazolyl group substituted with 0-1 —R^{B} groups, or a thiadiazolyl group substituted with 0-1-R^{B} groups.

24. The compound of any one of embodiments 1-18, wherein Cy^{B} is selected from the group consisting of:

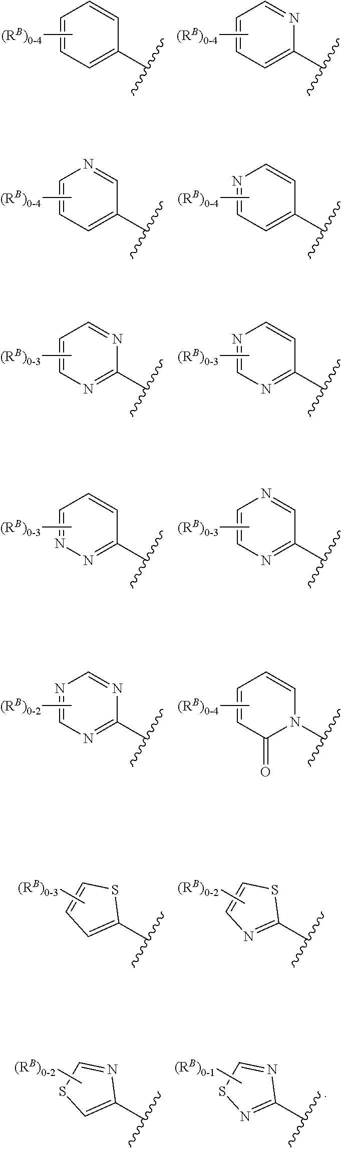

25. The compound ofany one of embodiments 1-18, wherein Cy^{B} is:

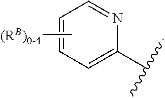

26. The compound of any one of embodiments 1-18, wherein Cy^{B} is

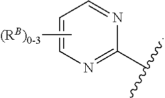

27. The compound of any one of embodiments 1-18, wherein Cy^{B} is

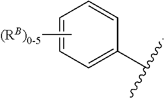

28. The compound of any one of embodiments 1-16, wherein Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups.

29. The compound of embodiment 28, wherein Cy^{B} and R^{x}, together with their intervening atoms, form a 6- to 12-membered spirocyclic ring system selected from:

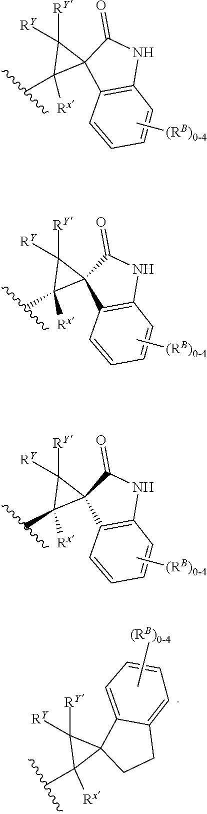

30. The compound of any one of the preceding embodiments, wherein each R^{B} is independently selected from oxo, halogen, —CN, —NO₂, —N(R)₂, —N(R)C(O)₂R, —OR, or an optionally substituted group selected from C1-6 aliphatic or a 5-membered heteroaryl having 1-4 heteroatoms independently selected from oxygen, nitrogen, and sulfur.

31. The compound of any one of the preceding embodiments, wherein substituents on an optionally substituted R^{B} group are independently selected from oxo, halogen, and —(CH₂)₀₋₄OR^{∘}, wherein R^{∘} is as defined above and described in classes and subclasses herein.

32. The compound of any one of the preceding embodiments, wherein each of R^{x} and R^{x′} is independently selected from hydrogen and halogen.

33. The compound of any one of the preceding embodiments, wherein each of R^{x} and R^{x′} is hydrogen.

34. The compound of any one of the preceding embodiments, wherein one of R^{x} and R^{x′} is hydrogen and the other is halogen.

35. The compound of any one of the preceding embodiments, wherein each of R^{Y} and R^{Y′} is independently selected from hydrogen and halogen.

36. The compound of any one of the preceding embodiments, wherein each of R^{Y} and R^{Y′} is hydrogen.

37. The compound of any one of the preceding embodiments, wherein L is an optionally substituted C₁₋₃ hydrocarbon chain, wherein 1-3 methylene units are optionally replaced with —O—, —NR^{z}—, —S—, or —So₂—.

38. The compound of any one of the preceding embodiments, wherein L is an optionally substituted C₁₋₃ hydrocarbon chain, wherein 1 methylene unit is optionally replaced with —O—, —NR—, —S—, or —SO₂—.

39. The compound of any one of the preceding embodiments, wherein L is an optionally substituted C₁ hydrocarbon chain.

40. The compound of embodiment 39, wherein Cy^{A} is bicyclic.

41. The compound of any one of the preceding embodiments, wherein L is an optionally substituted C2 hydrocarbon chain, wherein 1 methylene unit is optionally replaced with —NR^{z}— or —O—.

42. The compound of any one of the preceding embodiments, wherein L is an optionally substituted C₂ hydrocarbon chain, wherein the methylene unit connected to Cy^{A} is replaced with —NR^{z}— or —O—.

43. The compound of any one of the preceding embodiments, wherein L is an optionally substituted C₂ hydrocarbon chain, wherein the methylene unit connected to Cy^{A} is replaced with —NR^{z}—.

44. The compound of embodiment 43, wherein L is *—NHCH(Me)—, wherein * represents the point of attachment to Cy^{A}.

45. The compound of embodiment 43, wherein L is *—NHCH₂—, wherein * represents the point of attachment to Cy^{A}.

46. The compound of any one of embodiments 1-42, wherein L is an optionally substituted C2 hydrocarbon chain, wherein the methylene unit connected to Cy^{A} is replaced with —O—.

47. The compound of embodiment 46, wherein L is *—OCH(Me)—, wherein * represents the point of attachment to Cy^{A}.

48. The compound of embodiment 46, wherein L is *—OCH₂—, wherein * represents the point of attachment to Cy^{A}.

49. The compound of any one of the preceding embodiments, wherein L comprises a two-atom spacer between Cy^{A} and

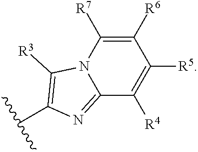

50. The compound of any one of the preceding embodiments, wherein L is an optionally substituted 5-membered saturated or partially unsaturated heterocyclene, having 1 heteroatom independently selected from oxygen, nitrogen, and sulfur.

51. The compound of any one of the preceding embodiments, wherein L is optionally substituted

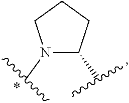

wherein * represents the point of attachment to Cy^{A}.

52. The compound of any one of embodiments 41-51, wherein Cy^{A} is monocyclic.

53. The compound of any one of the preceding embodiments, wherein optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

54. The compound of any one of the preceding embodiments, wherein L′ is a covalent bond or a methylene unit optionally substituted with —(CH₂)₀₋₄R^{∘}, wherein R^{∘} is independently as defined above and described in classes and subclasses herein.

55. The compound of any one of the preceding embodiments, wherein L′ is a covalent bond.

56. The compound of any one of the preceding embodiments, wherein each of R³, R⁴, R⁵, R⁶, and R⁷ is independently selected from hydrogen or L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{C}, or an optionally substituted group selected from C₁₋₆ aliphatic

57. The compound of any one of the preceding embodiments, wherein R³ is selected from hydrogen or L^{C}-R^{C}, wherein L^{C} is a covalent bond and R^{C} is halogen.

58. The compound of any one of the preceding embodiments, wherein R³ is hydrogen.

59. The compound of any one of the preceding embodiments, wherein R⁴ is selected from hydrogen or L^{C}-R^{C}, wherein L^{C} is selected from a covalent bond or an optionally substituted C₁₋₆ hydrocarbon chain, wherein 1 to 3 methylene units are optionally and independently replaced with —O— or —NR—; and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic.

60. The compound of any one of the preceding embodiments, wherein L^{C} is a covalent bond, R^{C} is Cy^{C} and Cy^{C} is a 5-membered saturated or partially unsaturated monocyclic heterocyclyl having 1-2 heteroatoms selected from oxygen, nitrogen, or sulfur.

61. The compound of any one of the preceding embodiments, wherein R⁴ is selected from the group consisting of:

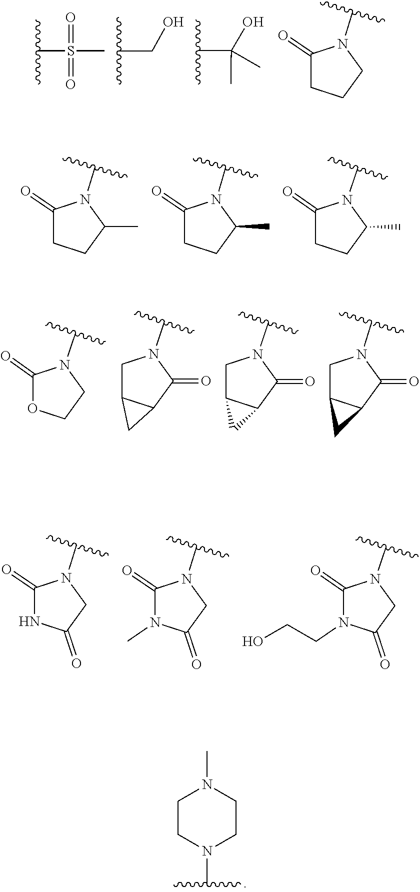

62. The compound of any one of the preceding embodiments, wherein optional substituents on a C₁₋₆ aliphatic group of R⁴ are selected from —(CH₂)₀₋₄R^{∘}, —(CH₂)₀₋₄OR^{∘}, —CN, —(CH₂)₀₋₄N(R^{∘})₂, and —(CH₂)₀₋₄C(O)OR^{∘}, wherein each R^{∘} is independently as defined above and described in classes and subclasses herein.

63. The compound of any one of the preceding embodiments, wherein Cy^{C} is selected from a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4-L^{D}-R^{D} groups.

64. The compound of any one of the preceding embodiments, wherein R^{D} is selected from oxo, halogen, —C(O)₂R, —N(R)₂, —OR, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

65. The compound of any one of the preceding embodiments, wherein optional substituents on R^{D} are selected from halogen, —(CH₂)₀₋₄R^{∘}, —(CH₂)₀₋₄OR^{∘}, —(CH₂)₀₋₄N(R^{∘})₂, —(CH₂)₀₋₄C(O)OR^{∘}, and —OP(O)(OR^{∘})₂, wherein each R^{∘} is independently as defined above and described in classes and subclasses herein.

66. The compound of any one of the preceding embodiments, wherein L^{D} is a covalent bond.

67. The compound of any one of the preceding embodiments, wherein R⁵ is hydrogen.

68. The compound of any one of embodiments 1-66, wherein R⁵ is L^{C}-R^{C}, wherein L^{C} is a covalent bond and R^{C} is Cy^{C}.

69. The compound of any one of the preceding embodiments, wherein Cy^{C} is a cyclopropyl group.

70. The compound of any one of the preceding embodiments, wherein R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆ aliphatic group.

71. The compound of any one of the preceding embodiments, wherein Cy^{C} is a cyclopropyl group substituted with 0-4 L^{D}-R^{D} groups.

72. The compound of any one of the preceding embodiments, wherein L^{C} is a covalent bond, and wherein R^{C} is an optionally substituted C₁₋₆ aliphatic group.

73. The compound of any one of the preceding embodiments, wherein Cy^{C} is an unsubstituted cyclopropyl group.

74. The compound of any one of the preceding embodiments, wherein L^{D} is a covalent bond and R^{D} is selected from halogen and optionally substituted C₁₋₆ aliphatic.

75. The compound of any one of the preceding embodiments, wherein R⁷ is selected from hydrogen or L^{C}-R^{C}, wherein L^{C} is a covalent bond, and wherein R^{C} is Cy^{C}.

76. The compound of any one of the preceding embodiments, wherein R⁷ is hydrogen.

77 The compound of any one of the preceding embodiments, wherein Cy^{C} is:

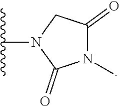

78. The compound of any one of the preceding embodiments, wherein R⁸ is hydrogen.

79. The compound of any one of embodiments 1-77, wherein R⁸ is selected from —OR or an optionally substituted C₁₋₆ aliphatic group.

80. The compound of any one of the preceding embodiments, wherein the compound is of Formula (II):

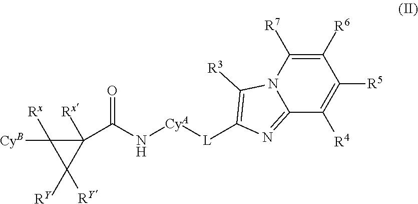

or a pharmaceutically acceptable salt thereof.

81. The compound of any one of the preceding embodiments, wherein the compound is of Formula (III-a), Formula (III-b), or Formula (III-c):

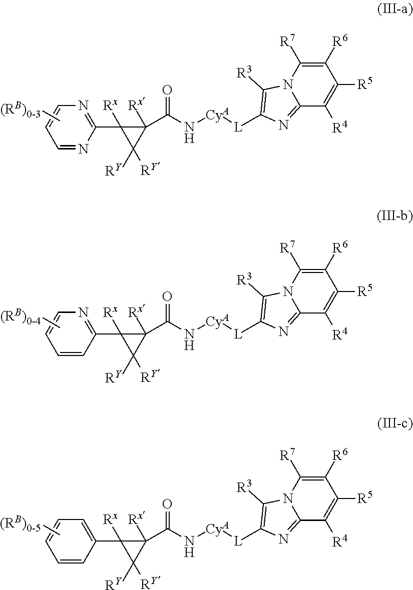

or a pharmaceutically acceptable salt thereof.

82. The compound of any one of the preceding embodiments, wherein the compound is of Formula (IV-a), Formula (IV-b), Formula (IV-c), Formula (IV-d), Formula (IV-e), or Formula (IV-e):

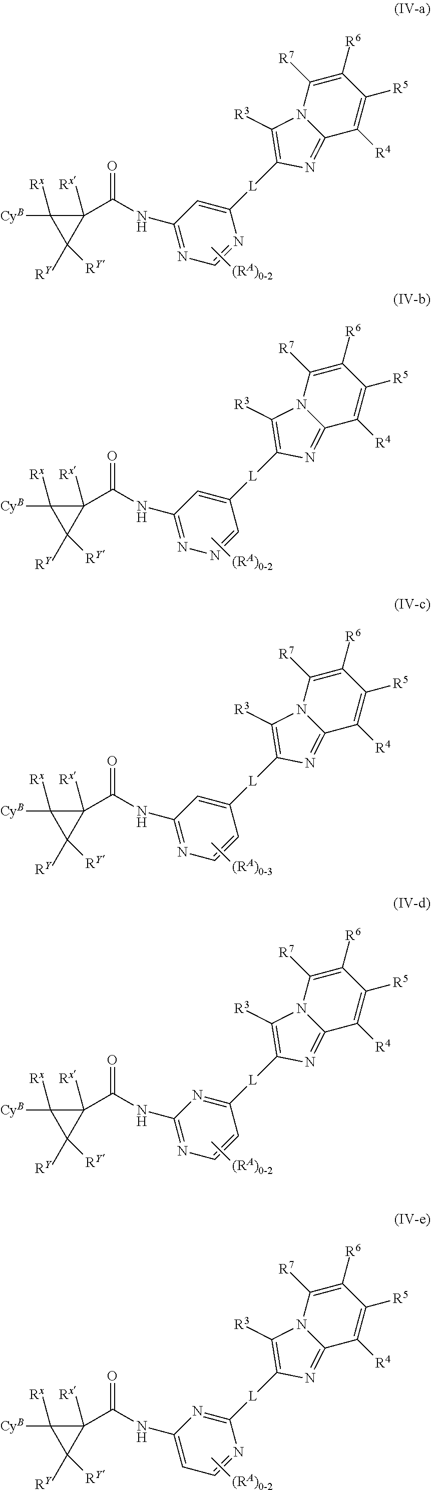

or a pharmaceutically acceptable salt thereof.

83. The compound of any one of the preceding embodiments, wherein the compound is of Formula (V-a), Formula (V-b), or Formula (V-c):

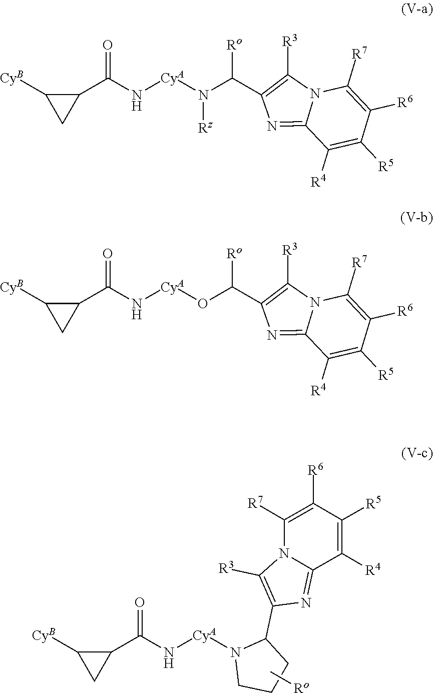

or a pharmaceutically acceptable salt thereof

84. The compound of any one of the preceding embodiments, wherein the compound is of Formula (V-a-1), Formula (V-b-1), or Formula (V-c-1):

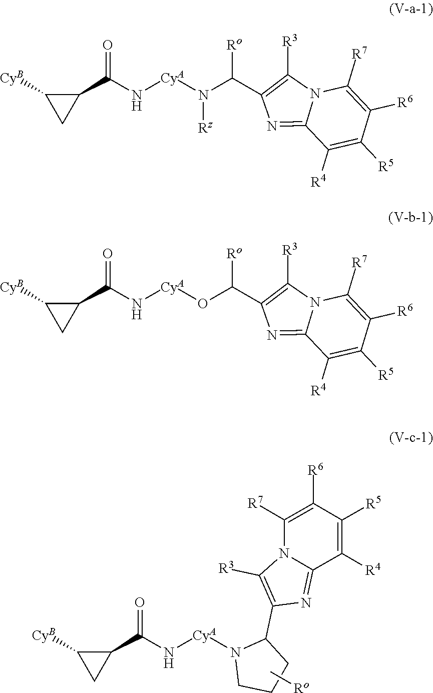

or a pharmaceutically acceptable salt thereof.

85. The compound of any one of the preceding embodiments, wherein the compound is of Formula (VI-a), Formula (VI-b), or Formula (VI-c):

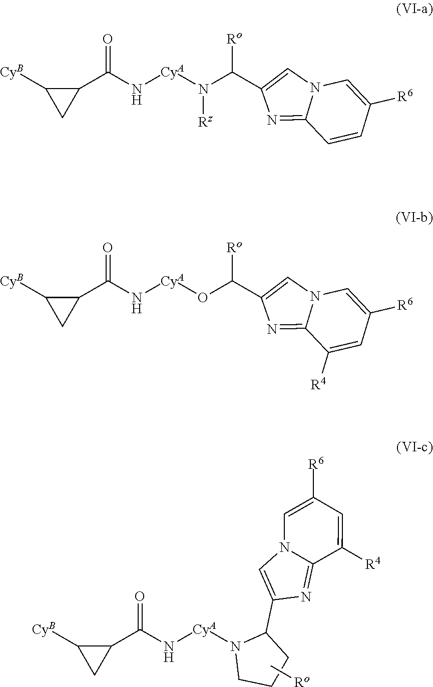

or a pharmaceutically acceptable salt thereof,

wherein each of Cy^{A}, Cy^{B}, R^{z}, and R^{∘} is defined and described in classes and subclasses herein, both singly and in combination;
R⁴ is L^{C}-R^{C}, wherein L^{C} is a covalent bond and R^{C} is Cy^{C}, wherein Cy^{C} is a 5-membered saturated or partially unsaturated monocyclic heterocyclyl having 1-2 heteroatoms selected from nitrogen, and wherein Cy^{C} is substituted with 0-4-L^{D}-R^{D} groups;
R⁶ is L^{C}-R^{C}, wherein L^{C} is a covalent bond and R^{C} is C₁₋₆ aliphatic or Cy^{C}, wherein Cy^{C} is cyclopropyl optionally substituted with halogen.

86. The compound of any one of the preceding embodiments, wherein R⁴ is a ring selected from:

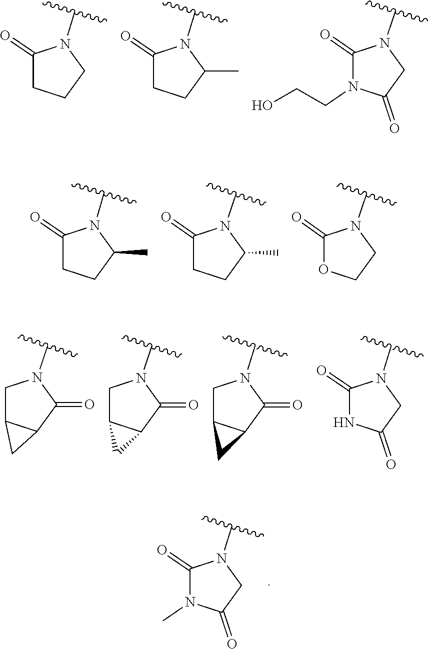

87. The compound of any one of the preceding embodiments, wherein R⁴ is a ring selected from:

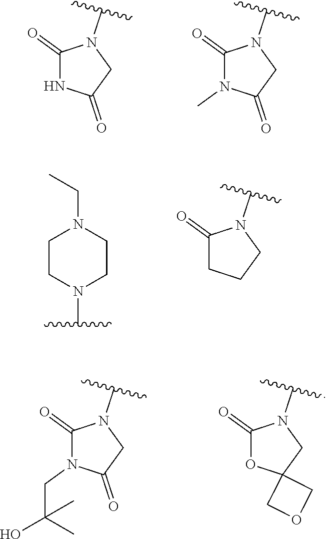

88. The compound of any one of the preceding embodiments, wherein the moiety:

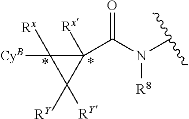

(including where one or more of R^{x}, R^{x′}, R^{Y}, or R^{Y′} is hydrogen) is in the relative trans configuration with respect to the Cy^{B} and amide group attached to the two stereocenters marked with an*.

89. The compound of embodiment 88, wherein the compound is a racemic mixture with respect to the two stereocenters marked with an *.

90. The compound of any one of embodiments 1-88, wherein the absolute stereochemistry of the moiety:

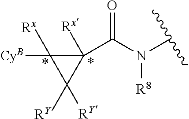

(including where one or more of R^{x}, R^{x′}, R^{Y}, or R^{Y′} is hydrogen) is as follows:

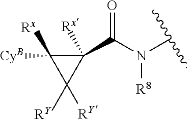

91. The compound of any one of embodiments 1-88, wherein the absolute stereochemistry of the moiety:

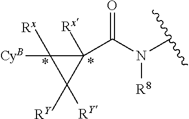

(including where one or more of R^{x}, R^{x′}, R^{Y}, or R^{Y′} is hydrogen) is as follows:

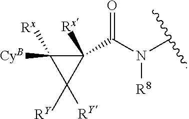

92. The compound of any one of the preceding embodiments, wherein each R^{∘} is independently selected from hydrogen, C₁₋₆ aliphatic, or —OH.

93. The compound of any one of the preceding embodiments, wherein R^{∘} of L is methyl.

94. The compound of any one of the preceding embodiments, wherein the compound is selected from compounds I-1 through I-679, or a pharmaceutically acceptable salt thereof.

95. A pharmaceutical composition comprising a compound of any one of the preceding embodiments.

96. The pharmaceutical composition comprising a compound of any one of the preceding embodiments, further comprising a pharmaceutically acceptable excipient.

97. The composition of embodiment 95 or 96, wherein the composition is suitable for oral administration.

98. A method of treating a plasma kallikrein-mediated disease or disorder using a compound or composition of any one of the preceding embodiments.

99. The method of embodiment 98, wherein the disease or disorder is hereditary angioedema.

100. The method of embodiment 98, wherein the disease or disorder is diabetic macular edema.

101. A method of treating hereditary angioedema comprising administering to a patient in need thereof a compound or composition of any one of the preceding embodiments.

102. A method of treating diabetic macular edema comprising administering to a patient in need thereof a compound or composition of any one of the preceding embodiments.

103. The method of any one of embodiment 99 or 101, wherein administration of the compound partially or completely inhibits, delays onset of, reduces severity of, and/or reduces incidence of one or more symptoms, features, and/or causes of a hereditary angioedema.

104. The method of embodiment 103, wherein the compound is administered orally.

1. A compound of Formula (I): 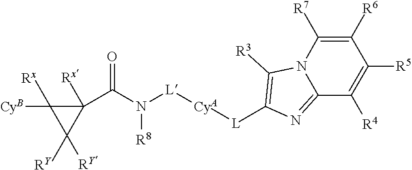
or a pharmaceutically acceptable salt thereof,
wherein:
Cy^{A} is a phenylene or a 5- to 6-membered monocyclic heteroarylene having 1-3 heteroatoms independently selected from oxygen, nitrogen, and sulfur, or a 7- to 12-membered bicyclic heteroarylene having 1-4 heteroatoms independently selected from oxygen, nitrogen, and sulfur, wherein Cy^{A} is substituted with 0-4 —R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
each R is independently hydrogen or an optionally substituted C₁₋₆ aliphatic group;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R^{z} is independently selected from hydrogen, —(CH₂)₀₋₃OR, —(CH₂)₀₋₃C(O)OR, or an optionally substituted C₁₋₆ aliphatic group;
L′ is a covalent bond or an optionally substituted C₁₋₃ hydrocarbon chain, wherein 1 to 3 methylene units are optionally and independently replaced with —O—, —NR^{z}—, —S—, —SO—, or SO₂—;
R⁶ is Cy^{C} and the latter is a cyclopropyl group substituted with 0-4 L^{D}-R^{D} groups; each R³, R⁴, R⁵, and R⁷ is independently selected from hydrogen or -L^{C}-R^{C}, wherein
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted group selected from C₁₋₆ aliphatic;
each Cy^{C} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{C} is substituted with 0-4-L^{D}-R^{D} groups;
each L^{D} is independently selected from a covalent bond or an optionally substituted C₁₋₆ hydrocarbon chain, wherein 1 to 3 methylene units are optionally and independently replaced with —O— or —NR—;
each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur; and
R⁸ is selected from hydrogen, —OR, or an optionally substituted C₁₋₆ aliphatic group.

2. The compound of claim 1, wherein L′ is a covalent bond.

3. The compound of claim 1, wherein the compound is of Formula (II): 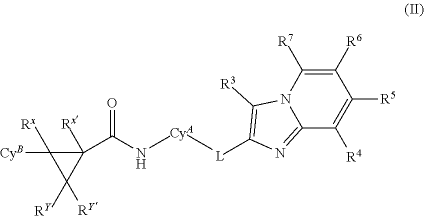
or a pharmaceutically acceptable salt thereof, wherein R³, R⁴, R⁵, R⁶, R⁷, R^{X}, R^{X′}, R^{Y}, R^{Y′}, Cy^{A}, Cy^{B} and L are defined for compounds of formula (I).

4. The compound of claim 1, wherein the compound is of Formula (III-a), Formula (III-b), or Formula (III-c): 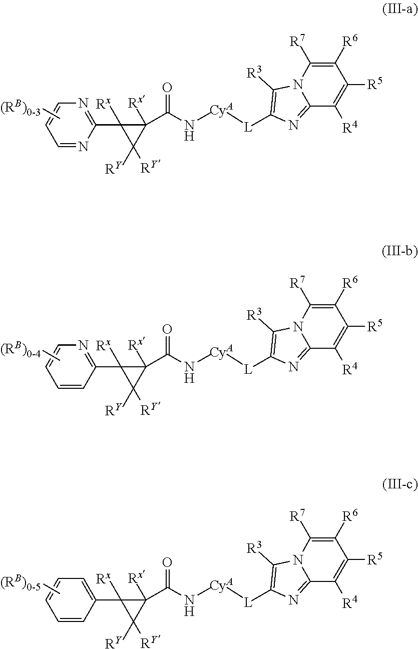
or a pharmaceutically acceptable salt thereof wherein R³, R⁴, R⁵, R⁶, R⁷, R^{B}, R^{X}, R^{X′}, R^{Y}, R^{Y′}, Cy^{A}, and L are defined for compounds of formula (I).

5. The compound of claim 1, wherein the compound is of Formula (IV-a), Formula (IV-b), Formula (IV-c), Formula (IV-d), Formula (IV-e), or Formula (IV-e): 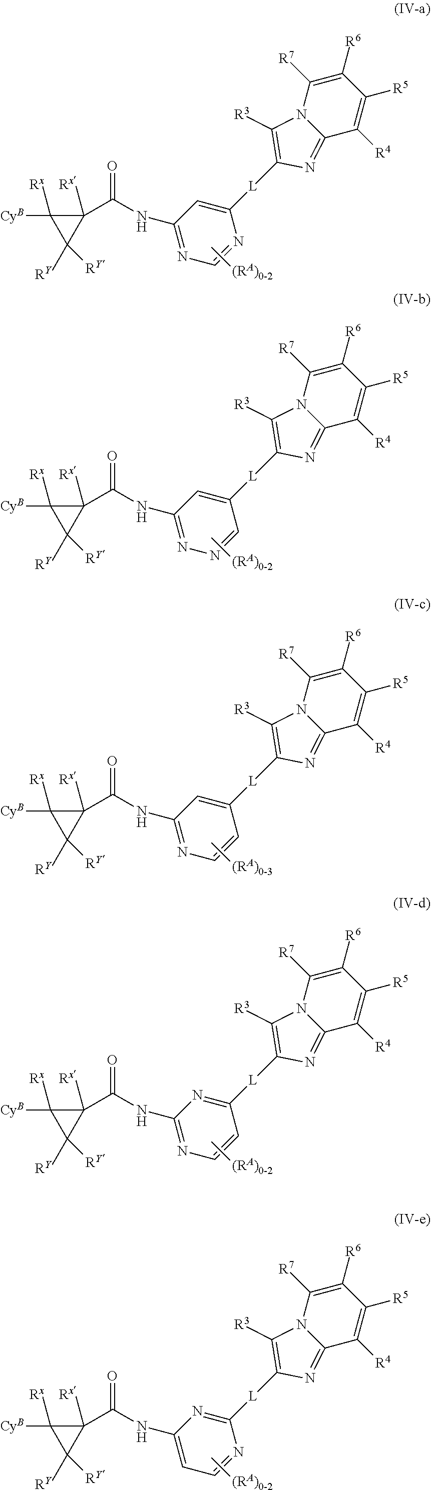
or a pharmaceutically acceptable salt thereof wherein R³, R⁴, R⁵, R⁶, R⁷, R^{X}, R^{X′}, R^{Y}, R^{Y′}, R^{A} and Cy^{B} are defined for compounds of formula (I).

6. The compound of claim 1, wherein the compound is of Formula (V-a), Formula (V-b), or Formula (V-c): 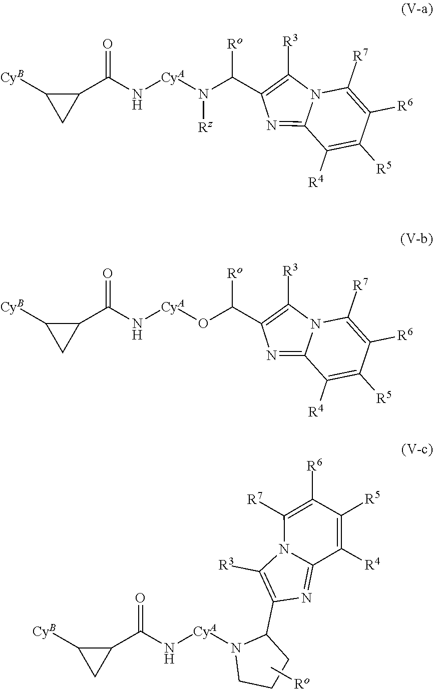
or a pharmaceutically acceptable salt thereof wherein R³, R⁴, R⁵, R⁶, R⁷, R^{X}, R^{X′}, R^{Y}, R^{Y′}, Cy^{A} and Cy^{B} are defined for compounds of formula (I).

7. The compound of claim 1, wherein the compound is of Formula (V-a-1), Formula (V-b-1), or Formula (V-c-1): 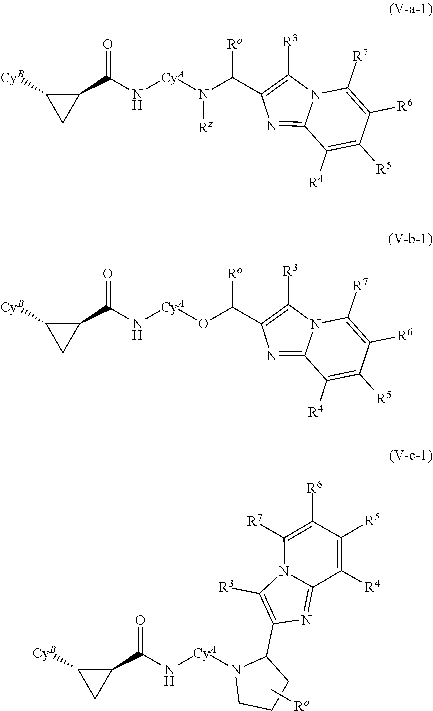
or a pharmaceutically acceptable salt thereof wherein R³, R⁴, R⁵, R⁶, R⁷, Cy^{A} and Cy^{B} are defined for compounds of formula (I), and R^{∘} is hydrogen or C₁₋₆ aliphatic.

8. The compound of claim 7, wherein the compound is of Formula (VI-a), Formula (VI-b), or Formula (VI-c): 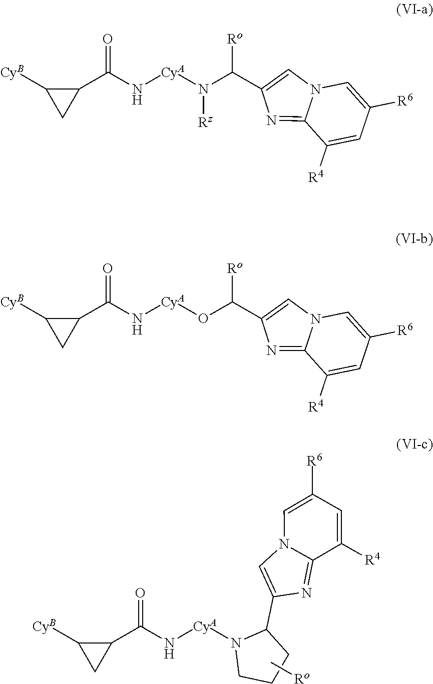
or a pharmaceutically acceptable salt thereof
wherein each of Cy^{A}, Cy^{B}, R^{z}, and R^{∘} is defined for compounds of formula (V);
R⁴ is L^{C}-R^{C}, wherein L^{C} is a covalent bond and R^{C} is Cy^{C}, wherein Cy^{C} is a 5-membered saturated or partially unsaturated monocyclic heterocyclyl having 1-2 heteroatoms selected from nitrogen, and wherein Cy^{C} is substituted with 0-4-L^{D}-R^{D} groups;
R⁶ is L^{C}-R^{C}, wherein L^{C} is a covalent bond and R^{C} is C₁₋₆ aliphatic or Cy^{C}, wherein Cy^{C} is cyclopropyl optionally substituted with halogen.

9. The compound of claim 1, wherein L is an optionally substituted C₁₋₃ hydrocarbon chain, wherein 1-3 methylene units are optionally replaced with —O—, —NR^{z}—, —S—, or —SO₂—.

10. The compound of claim 1, wherein L is *—NHCH(Me)—, wherein * represents the point of attachment to Cy^{A}.

11. The compound of a claim 1, wherein L is *—NHCH₂—, wherein * represents the point of attachment to Cy^{A}.

12. The compound of claim 1, wherein L is *—OCH(Me)—, wherein * represents the point of attachment to Cy^{A}.

13. The compound of claim 1, wherein L is *—OCH₂—, wherein * represents the point of attachment to Cy^{A}.

14. The compound of claim 1, wherein L comprises a two-atom spacer between Cy^{A} and

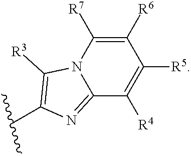

15. The compound of claim 1, wherein Cy^{A} is a 5- to 6-membered monocyclic heteroarylene having 1-3 heteroatoms independently selected from oxygen, nitrogen, and sulfur (for example a pyrimidinediyl or pyridinediyl, such as pyrimidinediyl), wherein Cy^{A} is substituted with 0-4 —R^{A} groups (for example 0 or 1).

16. The compound of claim 15, wherein Cy^{A} is selected from the group consisting of: 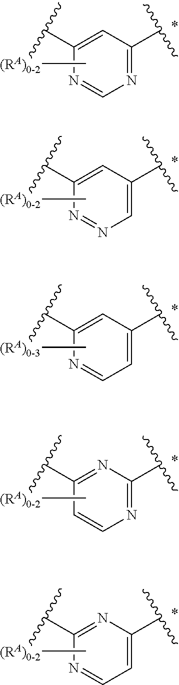
wherein * represents the point of attachment to L.

17. The compound of claim 1, wherein Cy^{A} is selected from the group consisting of: 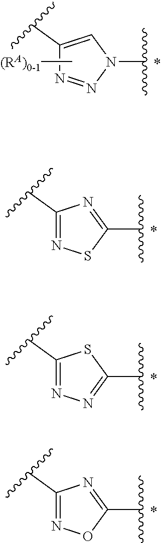
wherein * represents the point of attachment to L.

18. The compound of claim 1, wherein each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur, in particular an optionally substituted group selected from C₁₋₆ aliphatic, such as methyl.

19. The compound of claim 1, wherein Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 —R^{B} groups (for example 0 or 1 groups) 5- to 6-membered heteroaryl having 1-3 heteroatoms independently selected from oxygen, nitrogen, and sulfur (such as pyridine or pyrimidine, in particular pyrimidine) substituted with 0-4 —R^{B} groups.

20. The compound of claim 1, wherein Cy^{B} is selected from the group consisting of:

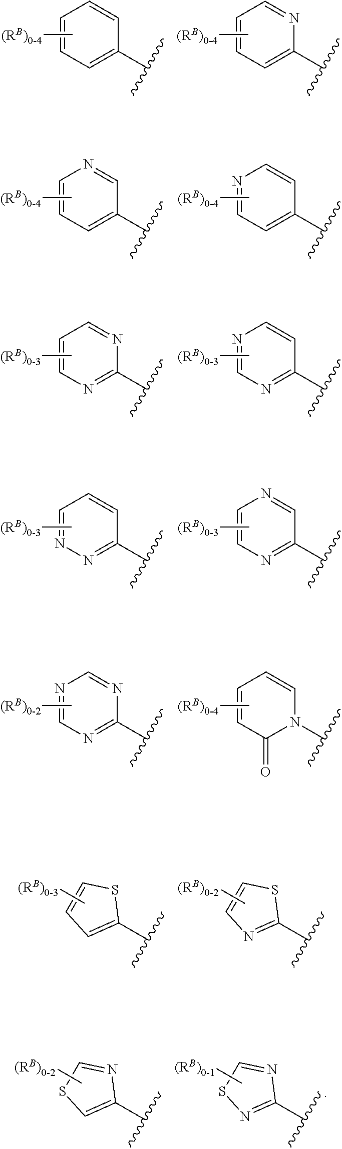

21. The compound of claim 1, wherein each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur, for example where the optionally substituted group is selected from C₁₋₆ aliphatic, such as methyl.

22. The compound of claim 1, wherein each of R^{x} and/or R^{x′} is hydrogen.

23. The compound of claim 1, wherein each of R^{Y} and/or R^{Y′} is hydrogen.

24. The compound of claim 1, wherein each of R³, R⁴, R⁵, R⁶, and R⁷ is independently selected from hydrogen or L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂,
—N(R)C(O)R, —N(R)C(O)₂R, —N(R)S(O)₂R, —S(O)₂R, —S(O)₂N(R)₂, Cy^{C}, or an optionally substituted group selected from C₁₋₆ aliphatic.

25. The compound of claim 1, wherein R³ is hydrogen.

26. The compound of claim 1, wherein R⁴ is selected from the group consisting of:

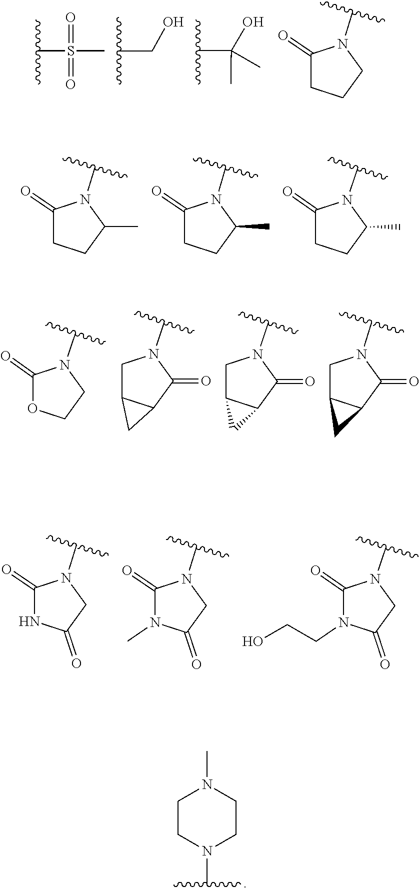

27. The compound of claim 1, wherein R⁵ is hydrogen.

28. The compound of claim 1, wherein R⁶ is Cy^{C} and the latter is a cyclopropyl group substituted with methyl or halogen.

29. The compound of claim 1, wherein Cy^{C} is an unsubstituted cyclopropyl group.

30. The compound of claim 1, wherein R⁷ is hydrogen.

31. The compound of claim 1, wherein the compound is selected from those in
(1S,2S)—N-(6-(((8-(4-acetylpiperazin-1-yl)-6-cyclopropylimidazo[1,2-a]pyridin-2-yl)methyl)amino)pyrimidin-4-yl)-2-(3-chlorophenyl)cyclopropane-1-carboxamide;
(1S,2S)-2-(3-chlorophenyl)-N-(6-(((6-cyclopropyl-8-(4-methylpiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-hydroxypiperidin-1-yl)imidazo[1,2-a]pyridin-2-yl)methyl)amino)pyrimidin-4-yl)cyclopropane-1-carboxamide;
(1S,2S)-2-(3-chlorophenyl)-N-(4-(((6-cyclopropyl-8-(3-(dimethylamino)azetidin-1-yl)imidazo[1,2-a]pyridin-2-yl)methyl)amino)pyridin-2-yl)cyclopropane-1-carboxamide;
(1S,2S)-2-(3-chlorophenyl)-N-(6-(((6-cyclopropyl-8-((1-methylpiperidin-4-yl)amino)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(methoxymethyl)-4-methylpiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(hydroxymethyl)-4-methylpiperazin-1-yl)imidazo[1,2-a]pyridin-2-yl)methyl)amino)pyrimidin-4-yl)cyclopropane-1-carboxamide;
methyl 4-(2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)-1-methylpiperazine-2-carboxylate;
(1S,2S)-2-(3-chlorophenyl)-N-(6-(((6-cyclopropyl-8-(3-methyl-4-oxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(1,1-dioxidothiomorpholino)imidazo[1,2-a]pyridin-2-yl)methyl)amino)pyrimidin-4-yl)cyclopropane-1-carboxamide;
(1S,2S)—N-(6-(((8-(1,4-diazabicyclo[3.2.1]octan-4-yl)-6-cyclopropylimidazo[1,2-a]pyridin-2-yl)methyl)amino)pyrimidin-4-yl)-2-(3-chlorophenyl)cyclopropane-1-carboxamide;
(1S,2S)-2-(3-chlorophenyl)-N-(6-(((6-cyclopropyl-8-(6-cyclopropyl-2,6-diazaspiro[3.3]heptan-2-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-((R)-3-(dimethylamino)pyrrolidin-1-yl).midazo [1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-((S)-3-(dimethylamino)pyrrolidin-1-yl).midazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(methylamino)pyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(dimethylamino)-2-oxopyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-(dimethylamino)-2-oxopyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(5-oxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-((6-cyclopropyl-8-(4-(2,2,2-trifluoroethyl)piperazin-1-yl)imidazo[1,2-a]pyridin-2-yl)methylamino)pyrimidin-4-yl)cyclopropane-1-carboxamide;
(1S,2S)-2-(3-chlorophenyl)-N-(4-(((6-cyclopropyl-8-(3-(trifluoromethyl)piperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-(4-methoxybenzyl)-2,4-dioxoimidazolidin-1-yl)imidazo[1,2-a]pyridin-2-yl)methyl)amino)pyridin-2-yl)cyclopropane-1-carboxamide;
(1S,2S)—N-(4-(((8-(1,4-diazabicyclo[3.2.1]octan-4-yl)-6-cyclopropylimidazo[1,2-a]pyridin-2-yl)methyl)amino)pyridin-2-yl)-2-(3-chlorophenyl)cyclopropane-1-carboxamide;
(1S,2S)-2-(3-chlorophenyl)-N-(4-(((6-cyclopropyl-8-(6-cyclopropyl-2,6-diazaspiro[3.3]heptan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-(dimethylamino)pyrrolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(oxetan-3-ylamino)imidazo[1,2-a]pyridin-2-yl)methyl)amino)pyridin-2-yl)cyclopropane-1-carboxamide;
(1S,2S)—N-(6-(((R)-1-(6-cyclopropyl-8-(7-oxo-2-oxa-6-azaspiro[3.4]octan-6-yl)imidazo[1,2-a]pyridin-2-yl)ethyl)amino)-2-methylpyrimidin-4-yl)-2-(4-methylpyrimidin-2-yl)cyclopropane-1-carboxamide;
rac-(1S*,2S*)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide;
(1R,2R)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide;
rac-(1S*,2S*)—N-(6-((8-(3-(2-(benzyloxy)ethyl)-2,4-dioxoimidazolidin-1-yl)-6-cyclopropylimidazo[1,2-a]pyridin-2-yl)methoxy)pyrimidin-4-yl)-2-(4-methylpyrimidin-2-yl)cyclopropane-1-carboxamide;
(1S,2S)—N-(6-((6-cyclopropyl-8-(2,4-dioxoimidazolidin-1-yl)imidazo[1,2-a]pyridin-2-yl)methoxy)pyrimidin-4-yl)-2-(4-methylpyrimidin-2-yl)cyclopropanecarboxamide;
rac-(1S*,2S*)—N-(6-((6-cyclopropyl-8-(3-(2-hydroxyethyl)-2,4-dioxoimidazolidin-1-yl)imidazo[1,2-a]pyridin-2-yl)methoxy)pyrimidin-4-yl)-2-(4-methylpyrimidin-2-yl)cyclopropane-1-carboxamide;
(1S,2S)—N-(4-((6-cyclopropyl-8-(3-methyl-2,4-dioxoimidazolidin-1-yl)imidazo[1,2-a]pyridin-2-yl)methoxy)-6-methylpyridin-2-yl)-2-(4-methylpyrimidin-2-yl)cyclopropane-1-carboxamide;
(1S,2S)—N-(5-(1-(6-cyclopropyl-8-(3-methyl-2,4-dioxoimidazolidin-1-yl)imidazo[1,2-a]pyridin-2-yl)ethoxy)-6-methylpyridazin-3-yl)-2-(4-methylpyrimidin-2-yl)cyclopropane-1-carboxamide;
(1S,2S)—N-(5-(1-(6-cyclopropyl-8-(2-oxooxazolidin-3-yl)imidazo[1,2-a]pyridin-2-yl)ethoxy)pyridazin-3-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-4-methoxypyridin-2-yl)-2-(4-methylpyrimidin-2-yl)cyclopropane-1-carboxamide;
(1S,2S)—N-(5-(1-(6-cyclopropyl-8-(2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide;
(1S,2S)—N-(5-((S)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide;
(1S,2S)—N-(5-((R)-1-(6-cyclopropyl-8-(3-methyl-2,4-dioxoimidazolidin-1-yl)imidazo[1,2-a]pyridin-2-yl)ethoxy)pyridazin-3-yl)-2-(4-methylpyrimidin-2-yl)cyclopropane-1-carboxamide;
rac-(1S*,2S*)-2-(3-chloro-2-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(3-chloro-4-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(3-chloro-5-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-methoxyphenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-cyanophenyl)-N-(6-(((6-cyclopropylimidazo[1,2-a]pyridin-2-yl)methyl)amino)pyrimidin-4-yl)cyclopropane-1-carboxamide;
rac-(1S*,2S*)—N-(6-(((6-cyclopropylimidazo[1,2-a]pyridin-2-yl)methyl)amino)pyrimidin-4-yl)-2-(2,5-dichlorophenyl)cyclopropane-1-carboxamide;
rac-(1S*,2S*)-2-(3-chloro-6-cyano-2-fluorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-(difluoromethyl)phenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-(trifluoromethyl)phenyl)-N-(6-(((6-cyclopropylimidazo[1,2-a]pyridin-2-yl)methyl)amino)pyrimidin-4-yl)cyclopropane-1-carboxamide;
rac-(1S*,2S*)—N-(6-(((6-cyclopropylimidazo[1,2-a]pyridin-2-yl)methyl)amino)pyrimidin-4-yl)-2-(3-methoxyphenyl)cyclopropane-1-carboxamide;
rac-(1S*,2S*)-2-(3-bromophenyl)-N-(6-(((6-cyclopropylimidazo[1,2-a]pyridin-2-yl)methyl)amino)pyrimidin-4-yl)cyclopropane-1-carboxamide;
(1R,2R)-2-(3-chlorophenyl)-N-(6-(((6-cyclopropylimidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
rac-(1S*,2S*)—N-(6-(((8-chloro-6-cyclopropylimidazo[1,2-a]pyridin-2-yl)methyl)amino)pyrimidin-4-yl)-2-(3-chlorophenyl)cyclopropane-1-carboxamide;
rac-(1S*,2S*)-2-(3-chlorophenyl)-N-(6-(((6-cyclopropyl-8-(methylsulfonyl)imidazo[1,2-a]pyridin-2-yl)methyl)amino)pyrimidin-4-yl)cyclopropane-1-carboxamide;
rac-ethyl 3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoate;
rac-(1S*,2S*)-2-(3-chlorophenyl)-N-(6-(((6-cyclopropyl-8-(3-hydroxyoxetan-3-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(3-fluorooxetan-3-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-((R)-4-hydroxy-2-oxopyrrolidin-1-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(4,4-dimethyl-2-oxopyrrolidin-1-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(3-methyl-2-oxopyrrolidin-1-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(2-methyl-3-oxomorpholino)imidazo[1,2-a]pyridin-2-yl)methyl)amino)pyrimidin-4-yl)cyclopropane-1-carboxamide;
rac-(1S*,2S*)—N-(6-(((3-chloro-6-cyclopropylimidazo[1,2-a]pyridin-2-yl)methyl)amino)pyrimidin-4-yl)-2-(3-chlorophenyl)cyclopropane-1-carboxamide;
rac-(1S*,2S*)-2-(3-chlorophenyl)-N-(6-(((6-cyclopropyl-8-(hydroxymethyl)imidazo[1,2-a]pyridin-2-yl)methyl)amino)pyrimidin-4-yl)cyclopropane-1-carboxamide, formic acid salt;
rac-(1S*,2S*)-2-(3-chlorophenyl)-N-(6-(((8-cyano-6-cyclopropylimidazo[1,2-a]pyridin-2-yl)methyl)amino)pyrimidin-4-yl)cyclopropane-1-carboxamide;
rac-3-(2-(((6-((1S*,2S*)-2-(3-chlorophenyl)cyclopropane-1-carboxamido)pyrimidin-4-yl)amino)methyl)-6-cyclopropylimidazo[1,2-a]pyridin-8-yl)propanoic acid;
rac-(1S*,2S*)-2-(3-chlorophenyl)-N-(6-(((6-cyclopropyl-8-(morpholinomethyl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide;
(1S,2S)-2-(3-chlorophenyl)-N-(6-((6-cyclopropyl-8-(3-oxomorpholino)imidazo[1,2-a]pyridin-2-yl)methylamino)pyrimidin-4-yl)cyclopropanecarboxamide;
(1R,2R)-2-(3-chlorophenyl)-N-(6-(((6-cyclopropyl-8-(3-oxomorpholino)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropylimidazo[1,2-a]pyridin-2-yl)methyl)(2-hydroxyethyl)amino)pyrimidin-4-yl)cyclopropane-1-carboxamide;
rac-(1S*,2S*)-2-(3-chlorophenyl)-N-(4-(((6-cyclopropylimidazo[1,2-a]pyridin-2-yl)methyl)amino)pyrimidin-5-yl)cyclopropane-1-carboxamide;
(1S,2S)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
(1R,2R)-2-(5-chloro-2-cyanophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
(1R,2R)-2-(5-chloro-2-cyanophenyl)-N-(4-(((6-cyclopropylimidazo[1,2-a]pyridin-2-yl)methyl)amino)pyridin-2-yl)cyclopropane-1-carboxamide;
(1S,2S)-2-(5-chloro-2-cyanophenyl)-N-(4-(((6-cyclopropylimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
rac-(1S*,2S*)-2-(3-chlorophenyl)-N-(4-(((6-cyclopropylimidazo[1,2-a]pyridin-2-yl)methyl)amino)-5-fluoropyridin-2-yl)cyclopropane-1-carboxamide, formic acid salt;
rac-(1S*,2S*)-2-(3-chlorophenyl)-N-(4-(((8-(2-cyanoethyl)-6-cyclopropylimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-methylpiperazin-1-yl)imidazo[1,2-a]pyridin-2-yl)methyl)amino)pyrimidin-2-yl)cyclopropane-1-carboxamide;
(1S,2S)-2-(3-chlorophenyl)-N-(4-(((6-cyclopropyl-8-((S)-2-methyl-5-oxopyrrolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((R)-2-methyl-5-oxopyrrolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1R,4S)-3-oxo-2-azabicyclo[2.2.1]heptan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1S,4R)-3-oxo-2-azabicyclo[2.2.1]heptan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1S,5R)-2-oxo-3-azabicyclo[3.1.0]hexan-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((1R,5S)-2-oxo-3-azabicyclo[3.1.0]hexan-3-yl)imidazo[1,2-a]pyridin-2-yl)methyl)amino)pyridin-2-yl)cyclopropane-1-carboxamide;
rac-(1S*,2S*)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(hydroxymethyl)imidazo[1,2-a]pyridin-2-yl)methyl)amino)pyridin-2-yl)cyclopropane-1-carboxamide;
rac-(1S*,2S*)-2-(5-chloro-2-cyanophenyl)-N-(4-(((6-cyclopropyl-8-(hydroxymethyl) imidazo[1,2-a]pyridin-2-yl)methyl)amino)pyridin-2-yl)cyclopropane-1-carboxamide;
rac-(1S*,2S*)-2-(3-chlorophenyl)-N-(5-(((6-cyclopropylimidazo[1,2-a]pyridin-2-yl)methyl)amino)pyridazin-3-yl)cyclopropane-1-carboxamide;
rac-(1S*,2S*)-2-(5-chloro-2-cyanophenyl)-N-(5-(((6-cyclopropylimidazo[1,2-a]pyridin-2-yl)methyl)amino)pyridazin-3-yl)cyclopropane-1-carboxamide;
rac-(1S*,2S*)-2-(3-chlorophenyl)-N-(5-((6-cyclopropylimidazo[1,2-a]pyridin-2-yl)methoxy)pyridazin-3-yl)cyclopropane-1-carboxamide;
rac-(1S*,2S*)-2-(3-chlorophenyl)-N-(6-((6-cyclopropylimidazo[1,2-a]pyridin-2-yl)methoxy)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-(pyrrolidin-1-yl)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-(methylthio)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-methoxypyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-(methoxymethyl)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-(methylsulfinyl)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)-2-(methylsulfonyl)pyrimidin-4-yl)cyclopropane-1-carboxamide;
rac-ethyl 4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylate;
rac-4-((1S*,2S*)-2-(3-chlorophenyl)cyclopropane-1-carboxamido)-6-(((6-cyclopropylimidazo[1,2-a]pyridin-2-yl)methyl)amino)pyrimidine-2-carboxylic acid;
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(3-fluorooxetan-3-yl)pyrimidin-4-yl)-2-(4-methylpyrimidin-2-yl)cyclopropane-1-carboxamide;
rac-(1S*,2S*)-2-(3-chlorophenyl)-N-(4-(((6-cyclopropylimidazo[1,2-a]pyridin-2-yl)methyl)amino)pyridin-2-yl)cyclopropane-1-carboxamide;
(1R,2R)-2-(3-chlorophenyl)-N-(4-(((6-cyclopropylimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropylimidazo[1,2-a]pyridin-2-yl)methyl)amino) pyridin-2-yl)cyclopropane-1-carboxamide;
rac-(1S*,2S*)-2-(3-chlorophenyl)-N-(4-(((6-cyclopropyl-8-(2-oxopyrrolidin-1-yl)imidazo[1,2-a]pyridin-2-yl)methyl)amino)pyridin-2-yl)cyclopropane-1-carboxamide;
rac-(1S*,2S*)-2-(3-chlorophenyl)-N-(4-(((8-cyano-6-cyclopropylimidazo[1,2-a]pyridin-2-yl)methyl)amino)pyridin-2-yl)cyclopropane-1-carboxamide;
rac-(1S*,2S*)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide;
(1S,2S)-2-(3-chlorophenyl)-N-(4-(((6-cyclopropylimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(2-hydroxypropan-2-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3,3-difluoroazetidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-fluoroazetidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(methylsulfonamido)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-methyl-2-oxopiperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(N-methylmethylsulfonamido)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-morpholinoimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-methyl-3-oxopiperazin-1-yl)imidazo[1,2-a]pyridin-2-yl)methyl)amino)pyridin-2-yl)cyclopropane-1-carboxamide;
rac-(1S*,2S*)-2-(5-chloro-2-cyanophenyl)-N-(4-(((6-cyclopropyl-8-(2-hydroxypropan-2-yl) imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(piperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-fluoroazetidin-3-yl)imidazo[1,2-a]pyridin-2-yl)methyl)amino)pyridin-2-yl)cyclopropane-1-carboxamide, formic acid salt;
(1S,2S)-2-(3-chlorophenyl)-N-(4-(((6-cyclopropyl-8-(3-hydroxyazetidin-3-yl)imidazo[1,2-a]pyridin-2-yl)methyl)amino)pyridin-2-yl)cyclopropane-1-carboxamide;
(1S,2S)—N-(4-(((8-(-azetidin-3-yl(hydroxy)methyl)-6-cyclopropylimidazo[1,2-a]pyridin-2-yl)methyl)amino)pyridin-2-yl)-2-(3-chlorophenyl)cyclopropane-1-carboxamide;
(1S,2S)-2-(4-chloropyridin-2-yl)-N-(4-(((6-cyclopropylimidazo[1,2-a]pyridin-2-yl)methyl)(2-hydroxyethyl)amino)pyridin-2-yl)cyclopropane-1-carboxamide;
(1S,2S)-2-(3-chlorophenyl)-N-(4-(((6-cyclopropyl-8-(4-methylpiperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(N-methylacetamido)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(6-methyl-2,6-diazaspiro[3.3]heptan-2-yl) imidazo[1,2-a]pyridin-2-yl)methyl)amino)pyridin-2-yl)cyclopropane-1-carboxamide;
(1S,2S)-2-(3-chlorophenyl)-N-(4-((1-(6-cyclopropylimidazo[1,2-a]pyridin-2-yl)-2,2,2-trifluoroethyl)amino)pyridin-2-yl)cyclopropane-1-carboxamide;
(1S,2S)—N-(4-(((8-acetamido-6-cyclopropylimidazo[1,2-a]pyridin-2-yl)methyl)amino)pyridin-2-yl)-2-(3-chlorophenyl)cyclopropane-1-carboxamide;
rac-(1S*,2S*)-2-(5-chloro-2-cyanophenyl)-N-(4-(((6-cyclopropyl-8-(4-methyl piperazin-1-yl)imidazo[1,2-a]pyridin-2-yl)methyl)amino)pyridin-2-yl)cyclopropane-1-carboxamide;
(1S,2S)-2-(3-chlorophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-fluoroazetidin-1-yl)imidazo[1,2-a]pyridin-2-yl)methyl)amino)pyrimidin-4-yl)cyclopropane-1-carboxamide;
methyl 2-(((6-((1S,2S)-2-(3-chlorophenyl)cyclopropane-1-carboxamido)pyrimidin-4-yl)amino) methyl)-6-cyclopropylimidazo[1,2-a]pyridine-8-carboxylate;
(1S,2S)-2-(3-chlorophenyl)-N-(6-(((6-cyclopropyl-8-((1-methyl-2-oxopyrrolidin-3-yl)oxy)imidazo[1,2-a]pyridin-2-yl)methyl)amino)pyrimidin-4-yl)cyclopropane-1-carboxamide;
(1S,2S)—N-(6-(((8-(benzyloxy)-6-cyclopropylimidazo[1,2-a]pyridin-2-yl)methyl)amino)pyrimidin-4-yl)-2-(3-chlorophenyl)cyclopropane-1-carboxamide;
(1S,2S)-2-(3-chlorophenyl)-N-(6-(((6-cyclopropyl-8-(3-oxopiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2-methoxypyridin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(N,N-dimethylsulfamoyl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-3-fluoroimidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-methyl-3-oxopiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-morpholinoimidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-oxothiomorpholino)imidazo[1,2-a]pyridin-2-yl)methyl)amino)pyrimidin-4-yl)cyclopropane-1-carboxamide;
tert-butyl (2-(((6-((1S,2S)-2-(3-chlorophenyl)cyclopropane-1-carboxamido)pyrimidin-4-yl)amino)methyl)-6-cyclopropylimidazo[1,2-a]pyridin-8-yl)carbamate;
(1S,2S)-2-(3-chlorophenyl)-N-(6-(((6-cyclopropyl-8-(3-methyl-2-oxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2-(trifluoromethyl)pyrrolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2-hydroxypropan-2-yl)imidazo[1,2-a]pyridin-2-yl)methyl)amino)pyrimidin-4-yl)cyclopropane-1-carboxamide;
(1S,2S)—N-(6-(((6-cyclopropyl-8-(methylsulfonyl)imidazo[1,2-a]pyridin-2-yl)methyl)(methyl)amino)-2-methylpyrimidin-4-yl)-2-(4-methylpyrimidin-2-yl)cyclopropane-1-carboxamide;
(1S,2S)-2-(3-chlorophenyl)-N-(6-(((6-cyclopropyl-8-(methylsulfonyl)imidazo[1,2-a]pyridin-2-yl)methyl)amino)pyrimidin-4-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(fluoromethyl)pyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(difluoromethyl)pyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-(trifluoromethyl)pyrimidin-2-yl)cyclopropane-1-carboxamide;
rac-(1S*,2S*)-2-(3-cyano-6-methylpyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide;
rac-(1S*,2S*)-2-(6-chloro-3-cyanopyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide;
rac-(1S*,2S*)-2-(4-chloropyrimidin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide.
(1S,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino) pyrimidin-4-yl)cyclopropane-1-carboxamide;
rac-(1S*,2S*)-2-(5-chloro-2-cyanophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-methyl-2,5-dioxoimidazolidin-1-yl)imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide;
rac-(1S*,2S*)-2-(4-chloropyridin-2-yl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2-oxooxazolidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropylimidazo[1,2-a]pyridin-2-yl)methyl)amino)pyrimidin-4-yl)-N-hydroxycyclopropane-1-carboxamide;
rac-(1S*,2S*)-2-(6-chloropyrimidin-4-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(4-chloropyrimidin-2-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloropyridazin-3-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-3-cyanothiophen-2-yl)-N-(6-(((6-cyclopropylimidazo[1,2-a]pyridin-2-yl)methyl)amino)pyrimidin-4-yl)cyclopropane-1-carboxamide;
rac-(1R*,2S*,3R*)-2-(3-chlorophenyl)-N-(6-(((6-cyclopropylimidazo[1,2-a]pyridin-2-yl)methyl)amino)pyrimidin-4-yl)-3-(hydroxymethyl)cyclopropane-1-carboxamide;
rac-(1S*,2S*)—N-(6-(((6-cyclopropylimidazo[1,2-a]pyridin-2-yl)methyl)amino)pyrimidin-4-yl)-2-(5-methoxy-2-oxopyridin-1(2H)-yl)cyclopropane-1-carboxamide;
rac-tert-butyl (4-((1S*,2S*)-2-((6-(((6-cyclopropylimidazo[1,2-a]pyridin-2-yl)methyl)amino)pyrimidin-4-yl)carbamoyl)cyclopropyl)-6-methoxypyridin-2-yl)carbamate;
rac-(1R*,2R*)-6′-chloro-N-(6-(((6-cyclopropylimidazo[1,2-a]pyridin-2-yl)methyl)amino)pyrimidin-4-yl)-2′,3′-dihydrospiro[cyclopropane-1,1′-indene]-2-carboxamide;
rac-(1S*,2S*)-2-(3-chlorophenyl)-N-(6-(((6-cyclopropylimidazo[1,2-a]pyridin-2-yl)methyl)amino)pyrimidin-4-yl)-1-fluorocyclopropane-1-carboxamide;
rac-(1S*,2S*)-2-(5-chlorothiophen-2-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(4-chlorothiophen-2-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(2-chloro-6-methoxypyridin-4-yl)-N-(6-(((6-cyclopropylimidazo[1,2-a]pyridin-2-yl)methyl)amino)pyrimidin-4-yl)cyclopropane-1-carboxamide;
rac-tert-butyl (2-chloro-4-((1S*,2S*)-2-((6-(((6-cyclopropylimidazo[1,2-a]pyridin-2-yl)methyl) amino)pyrimidin-4-yl)carbamoyl)cyclopropyl)phenyl)carbamate;
rac-tert-butyl (3-chloro-5-((1S*,2S*)-2-((6-(((6-cyclopropylimidazo[1,2-a]pyridin-2-yl)methyl)amino)pyrimidin-4-yl)carbamoyl)cyclopropyl)phenyl)carbamate;
rac-(1S*,2S*)-2-(3-chloro-2-nitrophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-1H-indazol-3-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(4-chloropyridin-2-yl)-N-(6-(((6-cyclopropyl-8-(4-methylpiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(1-methylazetidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-methyl-2,4-dioxotetrahydropyrimidin-1(2H)-yl)imidazo[1,2-a]pyridin-2-yl)methyl)amino)pyrimidin-4-yl)cyclopropane-1-carboxamide;
rac-(1S*,2S*)-2-(4-chloropyridin-2-yl)-N-(6-(((6-cyclopropyl-8-(4-methyl-2-oxopiperazin-1-yl) imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(7-hydroxyhexahydropyrrolo[1,2-a]pyrazin-2(1H)-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(7,7-difluorohexahydropyrrolo[1,2-a]pyrazin-2(1H)-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-hydroxy-1-methylazetidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(5,5-dimethyl-2-oxooxazolidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(hexahydropyrrolo[1,2-a]pyrazin-2(1H)-yl) imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-cyclopropylpiperazin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-(oxetan-3-yl)piperazin-1-yl)imidazo[1,2-a]pyridin-2-yl)methyl)amino)pyrimidin-4-yl)cyclopropane-1-carboxamide;
rac-(1S*,2S*)—N-(6-(((6-cyclopropylimidazo[1,2-a]pyridin-2-yl)methyl)amino)pyrimidin-4-yl)-2-(4-methoxypyridin-2-yl)cyclopropane-1-carboxamide;
rac-(1S*,2S*)-2-(2-cyano-5-methoxyphenyl)-N-(4-(((6-cyclopropylimidazo[1,2-a]pyridin-2-yl)methyl)amino)pyridin-2-yl)cyclopropane-1-carboxamide;
(1S,2S)—N-(6-(((8-acetyl-6-cyclopropylimidazo[1,2-a]pyridin-2-yl)methyl)amino)pyrimidin-4-yl)-2-(3-chlorophenyl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)pyridin-2-yl)-2-(4-methylpyrimidin-2-yl)cyclopropane-1-carboxamide;
(1S,2S)-2-(3-chlorophenyl)-N-(4-(((6-cyclopropyl-8-(3-methyl-2,4-dioxoimidazolidin-1-yl)imidazo[1,2-a]pyridin-2-yl)methyl)amino)pyridin-2-yl)cyclopropane-1-carboxamide;
rac-(1S*,2S*)-2-(6-chloropyrimidin-4-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloropyridazin-3-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloro-3-cyanothiophen-2-yl)-N-(4-(((6-cyclopropylimidazo[1,2-a]pyridin-2-yl)methyl)amino)pyridin-2-yl)cyclopropane-1-carboxamide;
rac-(1R*,2S*,3R*)-2-(3-chlorophenyl)-N-(4-(((6-cyclopropylimidazo[1,2-a]pyridin-2-yl)methyl)amino)pyridin-2-yl)-3-(hydroxymethyl)cyclopropane-1-carboxamide;
rac-(1S*,2S*)—N-(4-(((6-cyclopropylimidazo[1,2-a]pyridin-2-yl)methyl)amino)pyridin-2-yl)-2-(4-methoxypyridin-2-yl)cyclopropane-1-carboxamide;
rac-tert-butyl (4-((1S*,2S*)-2-((4-(((6-cyclopropylimidazo[1,2-a]pyridin-2-yl)methyl)amino)pyridin-2-yl)carbamoyl)cyclopropyl)-6-methoxypyridin-2-yl)carbamate;
rac-(1S*,2S*)-2-(3-chlorophenyl)-N-(4-(((6-cyclopropylimidazo[1,2-a]pyridin-2-yl)methyl)amino)pyridin-2-yl)-1-fluorocyclopropane-1-carboxamide;
rac-(1S*,2S*)-2-(5-chlorothiophen-2-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(4-chlorothiophen-2-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(2-chloropyridin-4-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloropyridin-3-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(2-amino-5-chlorophenyl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloro-2-nitrophenyl)-N-(4-(((6-cyclopropylimidazo[1,2-a]pyridin-2-yl)methyl)amino)pyridin-2-yl)cyclopropane-1-carboxamide;
rac-(1S*,2S*)-2-(2-cyano-5-methoxyphenyl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloro-1H-indazol-3-yl)-N-(4-(((6-cyclopropylimidazo[1,2-a]pyridin-2-yl)methyl)amino)pyridin-2-yl)cyclopropane-1-carboxamide;
tert-butyl 3-(2-(((2-((1S,2S)-2-(3-chlorophenyl)cyclopropane-1-carboxamido)pyridin-4-yl) amino)methyl)-6-cyclopropylimidazo[1,2-a]pyridin-8-yl)azetidine-1-carboxylate;
(1S,2S)-2-(3-chlorophenyl)-N-(4-(((6-cyclopropyl-8-(3-fluoro-1-methylazetidin-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(7-hydroxyhexahydropyrrolo[1,2-a]pyrazin-2(1H)-yl)imidazo[1,2-a]pyridin-2-yl)methyl)amino)pyridin-2-yl)cyclopropane-1-carboxamide;
1S,2S)-2-(3-chlorophenyl)-N-(4-(((6-cyclopropyl-8-(4-fluoro-1-methylpiperidin-4-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(7,7-difluorohexahydropyrrolo[1,2-a]pyrazin-2(1H)-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(hexahydropyrrolo[1,2-a]pyrazin-2(1H)-yl) imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-cyclopropylpiperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-(oxetan-3-yl)piperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-ethylpiperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(methylsulfonyl)imidazo[1,2-a]pyridin-2-yl)methyl)amino)pyridin-2-yl)cyclopropane-1-carboxamide;
1S,2S)-2-(3-chlorophenyl)-N-(4-(((6-cyclopropyl-8-(1-hydroxyethyl)imidazo[1,2-a]pyridin-2-yl)methyl)amino)pyridin-2-yl)cyclopropane-1-carboxamide;
(1S,2S)—N-(4-(((8-acetyl-6-cyclopropylimidazo[1,2-a]pyridin-2-yl)methyl)amino)pyridin-2-yl)-2-(3-chlorophenyl)cyclopropane-1-carboxamide;
(1S,2S)-2-(3-chlorophenyl)-N-(4-(((6-cyclopropyl-8-(2-oxooxazolidin-3-yl)imidazo[1,2-a]pyridin-2-yl)methyl)amino)pyridin-2-yl)cyclopropane-1-carboxamide;
methyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate;
ethyl 3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoate;
ethyl 2-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)acetate;
rac-(1S*,2S*)-2-(3-chlorophenyl)-N-(4-(((6-cyclopropyl-8-(3-hydroxyoxetan-3-yl)imidazo[1,2-a]pyridin-2-yl)methyl)amino)pyridin-2-yl)cyclopropane-1-carboxamide;
(1S,2S)-2-(3-chlorophenyl)-N-(2-(((6-cyclopropyl-8-(4-methylpiperazin-1-yl)imidazo[1,2-a]pyridin-2-yl)methyl)amino)pyridin-4-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-methoxypyrimidin-2-yl)-2-(4-methylpyrimidin-2-yl)cyclopropane-1-carboxamide;
(1S,2S)—N-(6-(((R)-1-(6-cyclopropyl-8-(2,5-dimethyl-6-oxo-2,5,7-triazaspiro[3.4]octan-7-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(2-(((R)-1-(6-cyclopropyl-8-(3-methyl-2,4-dioxoimidazolidin-1-yl)imidazo[1,2-a]pyridin-2-yl)ethyl)amino)-6-methylpyridin-4-yl)-2-(4-methylpyrimidin-2-yl)cyclopropane-1-carboxamide;
(1S,2S)—N-(6-chloro-5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclopropane-1-carboxamide;
(1S,2S)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1R,2R)-2-(4-chloropyridin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1S,2S)-2-(3-chlorophenyl)-N-(6-((2R,4R)-2-(6-cyclopropylimidazo[1,2-a]pyridin-2-yl)-4-hydroxypyrrolidin-1-yl)pyrimidin-4-yl)cyclopropane-1-carboxamide;
(1S,2S)-2-(3-chlorophenyl)-N-(6-((R)-2-(6-cyclopropylimidazo[1,2-a]pyridin-2-yl)pyrrolidin-1-yl)pyrimidin-4-yl)cyclopropane-1-carboxamide;
(1S,2S)—N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)-2-(4-methylpyrimidin-2-yl)cyclopropane-1-carboxamide;
(1S,2S)-2-(3-chlorophenyl)-N-(6-(((6-cyclopropyl-8-(4-methylpiperazin-1-yl)imidazo[1,2-a]pyridin-2-yl)methyl)amino)pyrazin-2-yl)cyclopropane-1-carboxamide;
rac-(1S*,2S*)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide;
(1S,2S)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide;
(1R,2R)-2-(3-amino-5-chlorophenyl)-N-(6-(((6-cyclopropylimidazo[1,2-a]pyridin-2-yl)methyl)amino)pyrimidin-4-yl)cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1R,2R)-2-(4-chloropyridin-2-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropyl-8-(3-methyl-2,4-dioxoimidazolidin-1-yl) imidazo[1,2-a]pyridin-2-yl)methyl)amino)pyrimidin-4-yl)cyclopropane-1-carboxamide;
(1S,2S)-2-(4-chloropyridin-2-yl)-N-(4-(((6-cyclopropylimidazo[1,2-a]pyridin-2-yl)methyl)amino)pyridin-2-yl)cyclopropane-1-carboxamide;
(1R,2R)-2-(4-chloropyridin-2-yl)-N-(4-(((6-cyclopropylimidazo[1,2-a]pyridin-2-yl)methyl)amino)pyridin-2-yl)cyclopropane-1-carboxamide;
(1S,2S)-6′-chloro-N-(4-(((6-cyclopropylimidazo[1,2-a]pyridin-2-yl)methyl)amino)pyridin-2-yl)-2′,3′-dihydrospiro[cyclopropane-1,1′-indene]-2-carboxamide;
(1R,2R)-6′-chloro-N-(4-(((6-cyclo propylimidazo[1,2-a]pyridin-2-yl)methyl)amino)pyridin-2-yl)-2′,3′-dihydrospiro[cyclopropane-1,1′-indene]-2-carboxamide;
(1S,2S)-2-(3-chlorophenyl)-N-(6-(((6-cyclopropyl-8-(N-methylacetamido)imidazo[1,2-a]pyridin-2-yl)methyl)amino)pyrimidin-4-yl)cyclopropane-1-carboxamide;
(1S,2S)—N-(6-(((8-acetamido-6-cyclopropylimidazo[1,2-a]pyridin-2-yl)methyl)amino)pyrimidin-4-yl)-2-(3-chlorophenyl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-(dimethylamino)ethoxy)pyrimidin-4-yl)-2-(4-methylpyrimidin-2-yl)cyclopropane-1-carboxamide;
rac-(1S*,2S*)-2-(2-amino-6-methoxypyridin-4-yl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(4-amino-3-chlorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(2-amino-5-chlorophenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(2-amino-3-chlorophenyl)-N-(6-(((6-cyclopropylimidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(1-hydroxyethyl)imidazo[1,2-a]pyridin-2-yl)methyl)amino)pyrimidin-4-yl)cyclopropane-1-carboxamide;
(1S,2S)-2-(3-chlorophenyl)-N-(4-((2R,4S)-2-(6-cyclopropylimidazo[1,2-a]pyridin-2-yl)-4-hydroxypyrrolidin-1-yl)pyridin-2-yl)cyclopropane-1-carboxamide;
(1S,2S)-2-(3-chlorophenyl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1S,2S)—N-(4-(((8-(azetidin-3-yl)-6-cyclopropylimidazo[1,2-a]pyridin-2-yl)methyl)amino)pyridin-2-yl)-2-(3-chlorophenyl)cyclopropane-1-carboxamide;
(1S,2S)-2-(3-chlorophenyl)-N-(6-(((6-cyclopropyl-8-(4-methylpiperazin-1-yl)imidazo[1,2-a]pyridin-2-yl)methyl)amino)-2-hydroxypyrimidin-4-yl)cyclopropane-1-carboxamide;
rac-(1S*,2S*)-2-(2-amino-6-methoxypyridin-4-yl)-N-(4-(((6-cyclopropylimidazo[1,2-a]pyridin-2-yl)methyl)amino)pyridin-2-yl)cyclopropane-1-carboxamide;
rac-(1S*,2S*)-2-(5-chloro-2-(1H-tetrazol-1-yl)phenyl)-N-(4-(((6-cyclopropylimidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-((2-hydroxyethoxy)methyl)imidazo[1,2-a]pyridin-2-yl)methyl)amino)pyridin-2-yl)cyclopropane-1-carboxamide;
rac-(1S*,2S*)-2-(5-chloro-2-cyanophenyl)-N-(4-(((6-cyclopropyl-8-((2-hydroxyethoxy)methyl) imidazo[1,2-a]pyridin-2-yl)methyl)amino)pyridin-2-yl)cyclopropane-1-carboxamide;
3-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)propanoic acid;
(1S,2S)-2-(3-chlorophenyl)-N-(4-(((6-cyclopropyl-8-(3-hydroxypropyl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(2-hydroxyethyl)imidazo[1,2-a]pyridin-2-yl)methyl)amino)pyridin-2-yl)cyclopropane-1-carboxamide;
(1S,2S)—N-(4-(((8-(aminomethyl)-6-cyclopropylimidazo[1,2-a]pyridin-2-yl)methyl)amino)pyridin-2-yl)-2-(3-chlorophenyl)cyclopropane-1-carboxamide;
rac-(1S*,2S*)-2-(3-chlorophenyl)-N-(4-(((6-cyclopropyl-8-(3-fluorooxetan-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropylimidazo[1,2-a]pyridin-2-yl)methyl)amino)-6-hydroxy-1,3,5-triazin-2-yl)cyclopropane-1-carboxamide;
(1S,2S)-2-(3-chlorophenyl)-N-(6-(((6-cyclopropylimidazo[1,2-a]pyridin-2-yl)methyl)amino)-2-oxo-2,3-dihydropyrimidin-4-yl)cyclopropane-1-carboxamide;
(1S,2S)—N-(6-((2R,4S)-4-amino-2-(6-cyclopropylimidazo[1,2-a]pyridin-2-yl)pyrrolidin-1-yl)pyrimidin-4-yl)-2-(3-chlorophenyl)cyclopropane-1-carboxamide;
2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxylic acid;
2-(((6-((1S,2S)-2-(3-chlorophenyl)cyclopropane-1-carboxamido)pyrimidin-4-yl)amino)methyl)-6-cyclopropyl-N,N-dimethylimidazo[1,2-a]pyridine-8-carboxamide;
(1S,2S)-2-(3-chlorophenyl)-N-(6-(((6-cyclopropyl-8-(morpholine-4-carbonyl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-methylpiperazine-1-carbonyl) imidazo[1,2-a]pyridin-2-yl)methyl)amino)pyrimidin-4-yl)cyclopropane-1-carboxamide;
2-(((6-((1S,2S)-2-(3-chlorophenyl)cyclopropane-1-carboxamido)pyrimidin-4-yl)amino)methyl)-6-cyclopropylimidazo[1,2-a]pyridine-8-carboxamide;
2-(((6-((1S,2S)-2-(3-chlorophenyl)cyclopropane-1-carboxamido)pyrimidin-4-yl)amino)methyl)-6-cyclopropyl-N-methylimidazo[1,2-a]pyridine-8-carboxamide;
(1S,2S)-2-(3-chlorophenyl)-N-(6-(((6-cyclopropyl-8-(piperazine-1-carbonyl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2-oxo-1,2-dihydropyridin-3-yl)imidazo[1,2-a]pyridin-2-yl)methyl)amino)pyrimidin-4-yl)cyclopropane-1-carboxamide;
(1S,2S)—N-(6-(((8-(azetidin-3-yloxy)-6-cyclopropylimidazo[1,2-a]pyridin-2-yl)methyl)amino)pyrimidin-4-yl)-2-(3-chlorophenyl)cyclopropane-1-carboxamide;
(1S,2S)-2-(3-chlorophenyl)-N-(6-(((6-cyclopropyl-8-(piperazin-1-yl)imidazo[1,2-a]pyridin-2-yl)methyl)amino)pyrimidin-4-yl)cyclopropane-1-carboxamide;
(1S,2S)—N-(6-(((8-amino-6-cyclopropylimidazo[1,2-a]pyridin-2-yl)methyl)amino)pyrimidin-4-yl)-2-(3-chlorophenyl)cyclopropane-1-carboxamide;
(1S,2S)-2-(3-chlorophenyl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide;
rac-(1S*,2S*)-2-(5-chloro-2-(2-methoxyethoxy)phenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(5-chloro-2-(difluoromethoxy)phenyl)-N-(6-(((6-cyclopropylimidazo[1,2-a]pyridin-2-yl)methyl)amino)pyrimidin-4-yl)cyclopropane-1-carboxamide;
rac-ethyl 3-(4-chloro-2-((1S*,2S*)-2-((6-(((6-cyclopropylimidazo[1,2-a]pyridin-2-yl)methyl)amino)pyrimidin-4-yl)carbamoyl)cyclopropyl)phenoxy)propanoate;
rac-(1S*,2S*)-2-(5-chloro-2-hydroxyphenyl)-N-(6-(((6-cyclopropylimidazo[1,2-a]pyridin-2-yl)methyl)amino)pyrimidin-4-yl)cyclopropane-1-carboxamide;
rac-(1S*,2S*)-2-(6-cyano-2-fluoro-3-methoxyphenyl)-N-(6-(((6-cyclopropylimidazo[1,2-a]pyridin-2-yl)methyl)amino)pyrimidin-4-yl)cyclopropane-1-carboxamide;
rac-3-(4-chloro-2-((1S*,2S*)-2-((6-(((6-cyclopropylimidazo[1,2-a]pyridin-2-yl)methyl)amino) pyrimidin-4-yl)carbamoyl)cyclopropyl)phenoxy)acetic acid;
rac-(1S*,2S*)-2-(5-chloro-2-(1H-tetrazol-1-yl)phenyl)-N-(6-(((6-cyclopropylimidazo[1,2-a]pyridin-2-yl)methyl)amino)pyrimidin-4-yl)cyclopropane-1-carboxamide;
(1S,2S)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-((1S,5R)-2-oxo-3-azabicyclo[3.1.0]hexan-3-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide;
(1R,2R)—N-(6-(((6-cyclopropyl-8-(methylsulfonyl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(methylsulfonyl)imidazo[1,2-a]pyridin-2-yl)methyl)amino) pyrimidin-4-yl)-2-(4-methylpyrimidin-2-yl)cyclopropane-1-carboxamide;
(1S,2S)-2-(4-chloropyridin-2-yl)-N-(6-(((6-cyclopropyl-8-(methylsulfonyl)imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropyl-8-(1,1-dioxidothiomorpholino)imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropyl-8-(2-oxo-3-azabicyclo[3.1.0]hexan-3-yl) imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropylimidazo[1,2-a]pyridin-2-yl)methyl)(2-hydroxyethyl)amino)pyrimidin-4-yl)cyclopropane-1-carboxamide;
rac-(1R *,2S*)-2-(3-chlorophenyl)-N-(6-(((6-cyclopropyl-8-(3-methyl-2,4-dioxoimidazolidin-1-yl)imidazo[1,2-a]pyridin-2-yl)methyl)amino)pyrimidin-4-yl)-2-fluorocyclopropane-1-carboxamide;
(1R,2R)-2-(3-chlorophenyl)-N-(6-(((6-cyclopropyl-8-(3-methyl-2,4-dioxoimidazolidin-1-yl).midazo [1,2-a]pyridin-2-yl)methyl)amino) pyrimidin-4-yl)-2-fluorocyclopropane-1-carboxamide;
(1S,2S)-2-(3-chlorophenyl)-N-(6-(((6-cyclopropyl-8-(3-methyl-2,4-dioxoimidazolidin-1-yl)imidazo[1,2-a]pyridin-2-yl)methyl)amino) pyrimidin-4-yl)-2-fluorocyclopropane-1-carboxamide;
rac-(1S*,2S*)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
(1S,2S)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
(1R,2R)-2-(5-chloro-2-cyanophenyl)-N-(6-(((6-cyclopropyl-8-(2-oxopyrrolidin-1-yl)imidazo[1,2-a]pyridin-2-yl)methyl)amino)pyrimidin-4-yl)cyclopropane-1-carboxamide;
rac-(1S*,2S*))-2-(5-chloro-2-cyanophenyl)-N-(6-(((6-cyclopropyl-8-(2-hydroxypropan-2-yl)imidazo[1,2-a]pyridin-2-yl)methyl)amino)pyrimidin-4-yl)cyclopropane-1-carboxamide;
(1S,2S)-2-(3-chlorophenyl)-N-(4-(((6-cyclopropylimidazo[1,2-a]pyridin-2-yl)methyl)amino)-6-methylpyridin-2-yl)cyclopropane-1-carboxamide;
(1S,2S)-2-(4-chloropyridin-2-yl)-N-(4-(((6-cyclopropylimidazo[1,2-a]pyridin-2-yl)methyl)amino)-6-methoxypyridin-2-yl)cyclopropane-1-carboxamide;
(1S,2S)-2-(3-chlorophenyl)-N-(4-(((6-cyclopropylimidazo[1,2-a]pyridin-2-yl)methyl)amino)-6-methoxypyridin-2-yl)cyclopropane-1-carboxamide;
(1S,2S)-2-(3-chlorophenyl)-N-(4-(((6-cyclopropylimidazo[1,2-a]pyridin-2-yl)methyl)amino)-6-(2-morpholinoethyl)pyridin-2-yl)cyclopropane-1-carboxamide;
rac-(1S*,2S*)-2-(5-chloro-2-cyanophenyl)-N-(4-(((6-cyclopropylimidazo[1,2-a]pyridin-2-yl)methyl)amino)-6-methylpyridin-2-yl)cyclopropane-1-carboxamide;
(1S,2S)-2-(4-chloropyridin-2-yl)-N-(4-(((6-cyclopropylimidazo[1,2-a]pyridin-2-yl)methyl)amino)-6-oxo-1,6-dihydropyridin-2-yl)cyclopropane-1-carboxamide;
rac-(1S*,2S*)-2-(3-chlorophenyl)-N-(4-(((6-cyclopropylimidazo[1,2-a]pyridin-2-yl)methyl)amino)-1,3,5-triazin-2-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)pyrazin-2-yl)cyclopropane-1-carboxamide;
rac-(1S*,2S*)-2-(3-chlorophenyl)-N-(4-(((6-cyclopropylimidazo[1,2-a]pyridin-2-yl)methyl)amino)pyrimidin-2-yl)cyclopropane-1-carboxamide;
rac-(1S*,2S*)-2-(3-chlorophenyl)-N-(2-(((6-cyclopropylimidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropylimidazo[1,2-a]pyridin-2-yl)methyl)amino)pyridazin-4-yl)cyclopropane-1-carboxamide;
2-(3-chlorophenyl)-2-cyano-N-(6-(((6-cyclopropylimidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropylimidazo[1,2-a]pyridin-2-yl)methyl)amino)pyrimidin-4-yl)-N-methylcyclopropane-1-carboxamide;
rac-(1S*,2S*)—N-(6-(((6-cyclopropylimidazo[1,2-a]pyridin-2-yl)methyl)amino)pyrimidin-4-yl)-2-(3-cyclopropylphenyl)cyclopropane-1-carboxamide;
rac-(1S*,2S*)-2-(5-chloro-2-cyanophenyl)-N-(6-((6-cyclopropylimidazo[1,2-a]pyridin-2-yl)(hydroxy)methyl)pyrimidin-4-yl)cyclopropane-1-carboxamide;
rac-(1S*,2S*)—N-(6-(((6-cyclopropyl-8-(3-methyl-2,4-dioxoimidazolidin-1-yl).midazo[1,2-a]pyridin-2-yl)methyl)amino)-2-(fluoromethyl)pyrimidin-4-yl)-2-(4-methylpyrimidin-2-yl)cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)(methyl)amino)-2-(methoxymethyl)pyrimidin-4-yl)-2-(4-methylpyrimidin-2-yl) cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(methoxymethyl)pyrimidin-4-yl)-2-(4-methylpyrimidin-2-yl)cyclopropane-1-carboxamide;
(1S,2S)—N-(6-(((6-cyclopropyl-8-(6-methyl-5,7-dioxo-4,6-diazaspiro[2.4]heptan-4-yl)imidazo[1,2-a]pyridin-2-yl)methyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)propyl)amino)-2-methylpyrimidin-4-yl)-2-(4-methylpyrimidin-2-yl)cyclopropane-1-carboxamide;
(1S,2S)—N-(6-(((6-cyclopropyl-8-(3-(2-hydroxy-2-methylpropyl)-2,4-dioxoimidazolidin-1-yl)imidazo[1,2-a]pyridin-2-yl)methyl)(methyl)amino)-2-methylpyrimidin-4-yl)-2-(4-methylpyrimidin-2-yl)cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)(ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(methylsulfonyl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-((1-(7-cyclopropyl-5-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide.
(1S,2S)—N-(6-((1-(6-cyclopropyl-8-(methylsulfonyl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-((1-(6-cyclopropyl-8-(3-methyl-2-oxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)amino)-2-methylpyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-methylpyrimidin-4-yl)-2-(4-methylpyrimidin-2-yl)cyclopropane-1-carboxamide.
(1S,2S)—N-(6-(((6-cyclopropyl-8-(2,4-dimethyl-3,5-dioxo-1,2,4-triazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(3-(2-hydroxy-2-methylpropyl)-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide;
(1S,2S)—N-(6-((1-(6-cyclopropyl-8-(3-methyl-2,4-dioxoimidazolidin-1-yl)imidazo[1,2-a]pyridin-2-yl)ethyl)(methyl)amino)pyrimidin-4-yl)-2-(4-methylpyrimidin-2-yl)cyclopropane-1-carboxamide;
(1S,2S)—N-(6-((1-(6-cyclopropyl-8-(3-methyl-2,4-dioxoimidazolidin-1-yl)imidazo[1,2-a]pyridin-2-yl)-2,2,2-trifluoroethyl)amino)pyrimidin-4-yl)-2-(4-methylpyrimidin-2-yl)cyclopropane-1-carboxamide;
benzyl N-((6-cyclopropyl-8-(3-methyl-2,4-dioxoimidazolidin-1-yl)imidazo[1,2-a]pyridin-2-yl)methyl)-N-(6-((1S,2S)-2-(4-methylpyrimidin-2-yl)cyclopropane-1-carboxamido)pyrimidin-4-yl)glycinate;
(1S,2S)—N-(6-(((6-cyclopropyl-8-(6-oxo-2,5-dioxa-7-azaspiro[3.4]octan-7-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-((1-(6-cyclopropyl-8-(3-methyl-2,4-dioxoimidazolidin-1-yl)imidazo[1,2-a]pyridin-2-yl)-2-methoxyethyl)amino)pyrimidin-4-yl)-2-(4-methylpyrimidin-2-yl)cyclopropane-1-carboxamide;
(1S,2S)—N-(6-(((6-(1-fluorocyclopropyl)-8-(3-methyl-2,4-dioxoimidazolidin-1-yl)imidazo[1,2-a]pyridin-2-yl)methyl)amino) pyrimidin-4-yl)-2-(4-methylpyrimidin-2-yl)cyclopropane-1-carboxamide;
(1S,2S)—N-(6-(((6-cyclopropyl-8-(2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide;
(1S,2S)—N-(6-(((6-cyclopropyl-8-(2-oxooxazolidin-3-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(2-oxopyrrolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(3-(oxetan-3-yl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-3-fluoro-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropylimidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(3-(2-(2-methoxyethoxy)ethyl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(4-ethylpiperazin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(3-cyclopropyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((6-cyclopropyl-8-(3-ethyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(4-methyl-3,5-dioxo-1,2,4-triazolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide;
rac-(1S*,2S*)—N-(6-(((6-cyclopropyl-8-(2,4-dioxo-3-(2,2,2-trifluoroethyl)imidazolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(2-oxopyrrolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(2-oxooxazolidin-3-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl-d2)amino)pyrimidin-4-yl)-2-(4-methylpyrimidin-2-yl)cyclopropane-1-carboxamide;
rac-(1S*,2S*)—N-(6-(((6-cyclopropyl-8-(2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyridin-2-yl)cyclopropane-1-carboxamide;
(1S,2S)-2-(4-chloropyridin-2-yl)-N-(6-(((6-cyclopropyl-8-(2-oxoimidazolidin-1-yl)imidazo[1,2-a]pyridin-2-yl)methyl)amino)pyrimidin-4-yl)cyclopropane-1-carboxamide;
(1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino) pyrimidin-4-yl)cyclopropane-1-carboxamide;
(1S,2S)-2-(3-chlorophenyl)-N-(6-(((6-cyclopropyl-8-(2,4-dioxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-(4-methoxybenzyl)-2,4-dioxoimidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(2,4-dioxo-3-(2,2,2-trifluoroethyl)imidazolidin-1-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-cyclopropyl-2,4-dioxoimidazolidin-1-yl) imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-((3-methyl-2,4-dioxoimidazolidin-1-yl)methyl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-hydroxy-1-methylpiperidin-4-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(3-fluoro-1-methylazetidin-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-fluoro-1-methylpiperidin-4-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-(4-methyl-2,5-dioxopiperazin-1-yl)imidazo[1,2-a]pyridin-2-yl)methyl)amino)pyrimidin-4-yl)cyclopropane-1-carboxamide;
rac-(1S*,2S*)-2-(3-chlorophenyl)-N-(6-(((6-cyclopropyl-8-(4H-1,2,4-triazol-4-yl)imidazo[1,2-a]pyridin-2-yl)methyl)amino)pyrimidin-4-yl) cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)(methyl)amino)-2-methoxypyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methoxypyrimidin-4-yl)-2-(4-methylpyrimidin-2-yl)cyclopropane-1-carboxamide;
(1S,2S)—N-(2-cyclopropyl-6-(((6-cyclopropyl-8-(2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide;
(1S,2S)—N-(6-((1-(8-(3-(2-(benzyloxy)ethyl)-2,4-dioxoimidazolidin-1-yl)-6-cyclopropylimidazo[1,2-a]pyridin-2-yl)ethyl)amino)-2-cyclopropylpyrimidin-4-yl)-2-(4-methylpyrimidin-2-yl)cyclopropane-1-carboxamide;
(1S,2S)—N-(2-cyclopropyl-6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(difluoromethyl)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)-5-fluoropyrimidin-4-yl)-2-(4-methylpyrimidin-2-yl)cyclopropane-1-carboxamide;
rac-(1R*,2S*)-5′-chloro-N-(6-(((6-cyclopropylimidazo[1,2-a]pyridin-2-yl)methyl)amino)pyrimidin-4-yl)-1′-methyl-2′-oxospiro[cyclopropane-1,3′-indoline]-2-carboxamide;
rac-(1S*,2S*)—N-(4-(((6-cyclopropylimidazo[1,2-a]pyridin-2-yl)methyl)amino)pyridin-2-yl)-2-(4-methylpyrimidin-2-yl)cyclopropane-1-carboxamide;
(1S,2S)—N-(6-(((R)-1-(6-cyclopropyl-8-(2-oxopyrrolidin-1-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-(7-methyl-6,8-dioxo-2-oxa-5,7-diazaspiro[3.4]octan-5-yl) imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-((dimethyl(oxo)-16-sulfanylidene)amino)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(3S,5R)-5-(6-cyclopropyl-8-(3-methyl-2,4-dioxoimidazolidin-1-yl)imidazo[1,2-a]pyridin-2-yl)-1-(6-((1S,2S)-2-(4-methylpyrimidin-2-yl)cyclopropane-1-carboxamido)pyrimidin-4-yl)pyrrolidin-3-yl propionate;
(1S,2S)—N-(6-((2R)-2-(6-cyclopropylimidazo[1,2-a]pyridin-2-yl)-4-hydroxy-4-methylpyrrolidin-1-yl)pyrimidin-4-yl)-2-(4-methylpyrimidin-2-yl)cyclopropane-1-carboxamide;
(1S,2S)—N-(6-(((R)-1-(6-cyclopropyl-8-((dimethyl(oxo)-16-sulfanylidene)amino)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(3,5,5-trimethyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((1R)-1-(6-cyclopropyl-8-(2-oxo-3-azabicyclo[3.1.0]hexan-3-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(5-methyl-6-oxo-2-oxa-5,7-diazaspiro[3.4]octan-7-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(6-oxo-2,5-dioxa-7-azaspiro[3.4]octan-7-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-(1-fluorocyclopropyl)-8-(3-methyl-2,4-dioxoimidazolidin-1-yl)imidazo[1,2-a]pyridin-2-yl)ethyl)amino)-2-(methoxymethyl)pyrimidin-4-yl)-2-(4-methylpyrimidin-2-yl)cyclopropane-1-carboxamide;
(1S,2S)—N-(2-(((R)-1-(6-cyclopropyl-8-(3-methyl-2,4-dioxoimidazolidin-1-yl)imidazo[1,2-a]pyridin-2-yl)ethyl)amino)-6-methylpyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(dimethylamino)pyrimidin-4-yl)-2-(4-methylpyrimidin-2-yl)cyclopropane-1-carboxamide;
(1S,2S)-2-(4-chloropyridin-2-yl)-N-(6-((2R,4S)-2-(6-cyclopropylimidazo[1,2-a]pyridin-2-yl)-4-(hydroxymethyl)pyrrolidin-1-yl)pyrimidin-4-yl)cyclopropane-1-carboxamide;
(1S,2S)—N-(6-(((R)-1-(6-cyclopropyl-8-((1S,5R)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-((1R,5S)-2-oxo-3-azabicyclo[3.1.0]hexan-3-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide;
(1R,2R)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methylthiazol-2-yl)cyclopropane-1-carboxamide;
(1R,2R)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1R,2R)—N-(6-(((S)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((S)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)-2-(3-chlorophenyl)-N-(6-(((6-cyclopropyl-8-((1R,5S)-2-oxo-3-azabicyclo[3.1.0]hexan-3-yl)imidazo[1,2-a]pyridin-2-yl)methyl)amino)pyrimidin-4-yl)cyclopropane-1-carboxamide;
(1S,2S)-2-(3-chlorophenyl)-N-(6-(((6-cyclopropyl-8-((1S,5R)-2-oxo-3-azabicyclo[3.1.0]hexan-3-yl) imidazo[1,2-a]pyridin-2-yl)methyl)amino)pyrimidin-4-yl)cyclopropane-1-carboxamide;
(1S,2S)—N-(2-cyclopropyl-6-((1-(6-cyclopropyl-8-(3-(2-hydroxyethyl)-2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
N-((6-cyclopropyl-8-(3-methyl-2,4-dioxoimidazolidin-1-yl)imidazo[1,2-a]pyridin-2-yl)methyl)-N-(6-((1S,2S)-2-(4-methylpyrimidin-2-yl)cyclopropane-1-carboxamido)pyrimidin-4-yl)glycine;
(1S,2S)—N-(6-(((6-cyclopropyl-8-(3-(2-hydroxyethyl)-2,4-dioxoimidazolidin-1-yl)imidazo[1,2-a]pyridin-2-yl)methyl)(methyl)amino)pyrimidin-4-yl)-2-(4-methylpyrimidin-2-yl)cyclopropane-1-carboxamide;
(1S,2S)-2-(3-chlorophenyl)-N-(6-(((6-cyclopropyl-8-(3-(2-hydroxyethyl)-2,4-dioxoimidazolidin-1-yl)imidazo[1,2-a]pyridin-2-yl)methyl)amino)pyrimidin-4-yl)cyclopropane-1-carboxamide;
(1S,2S)—N-(6-(((6-cyclopropyl-8-(3-(2-hydroxyethyl)-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
2-(3-(6-cyclopropyl-2-(((6-((1S,2S)-2-(4-methylpyrimidin-2-yl)cyclopropane-1-carboxamido) pyrimidin-4-yl)amino)methyl)imidazo[1,2-a]pyridin-8-yl)-2,5-dioxoimidazolidin-1-yl)acetic acid;
(3-(6-cyclopropyl-2-(((6-((1S,2S)-2-(4-methylpyrimidin-2-yl)cyclopropane-1-carboxamido)pyrimidin-4-yl)amino)methyl)imidazo[1,2-a]pyridin-8-yl)-2,5-dioxoimidazolidin-1-yl)methyl dihydrogen phosphate;
1-((((6-cyclopropyl-8-(3-methyl-2,4-dioxoimidazolidin-1-yl)imidazo[1,2-a]pyridin-2-yl)methyl)(6-((1S,2S)-2-(4-methylpyrimidin-2-yl) cyclopropane-1-carboxamido)pyrimidin-4-yl)carbamoyl)oxy)ethyl.isobutyrate;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(2-hydroxyethyl)pyrimidin-4-yl)-2-(4-methylpyrimidin-2-yl)cyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-(hydroxymethyl)pyrimidin-4-yl)-2-(4-methylpyrimidin-2-yl)cyclopropane-1-carboxamide;
(1S,2S)—N-(6-((2R,4S)-4-hydroxy-2-(6-((1S*,2S*)-2-methylcyclopropyl)imidazo[1,2-a]pyridin-2-yl)pyrrolidin-1-yl)pyrimidin-4-yl)-2-(4-methylpyrimidin-2-yl)cyclopropane-1-carboxamide;
rac-(1S*,2S*)—N-(6-((2R,4S)-2-(6-cyclopropyl-8-(2-oxooxazolidin-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide;
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(4-methylpyridin-2-yl)cyclopropane-1-carboxamide;
rac-(1S*,2S*)—N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide;
rac-(1S*,2S*)—N-(6-((2R,4S)-2-(6-cyclopropyl-8-(2,4-dioxoimidazolidin-1-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide;
(1S,2S)-2-(4-chloropyridin-2-yl)-N-(6-((2R,4S)-2-(6-cyclopropyl-8-(2,4-dioxoimidazolidin-1-yl)imidazo[1,2-a]pyridin-2-yl)-4-hydroxypyrrolidin-1-yl)pyrimidin-4-yl)cyclopropane-1-carboxamide;
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(4-methylpyrimidin-2-yl)cyclopropane-1-carboxamide;
rac-(1S*,2S*)-2-(5-chloro-2-cyanophenyl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
(1S,2S)-2-(4-chloropyridin-2-yl)-N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)cyclopropane-1-carboxamide;
(1S,2S)-2-(3-chlorophenyl)-N-(6-((2R,4S)-2-(6-cyclopropyl-8-(2-oxooxazolidin-3-yl)imidazo[1,2-a]pyridin-2-yl)-4-hydroxypyrrolidin-1-yl)pyrimidin-4-yl)cyclopropane-1-carboxamide;
(1S,2S)-2-(3-chlorophenyl)-N-(6-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-4-yl)cyclopropane-1-carboxamide;
rac-(1S*,2S*)-2-(4-chloropyrimidin-2-yl)-N-(6-((2R,4S)-2-(6-cyclopropylimidazo[1,2-a]pyridin-2-yl)-4-hydroxypyrrolidin-1-yl)pyrimidin-4-yl)cyclopropane-1-carboxamide;
rac-(1S*,2S*)—N-(6-((2R,4S)-2-(6-cyclopropyl-8-(2-oxo-3-azabicyclo[3.1.0]hexan-3-yl)imidazo[1,2-a]pyridin-2-yl)-4-hydroxypyrrolidin-1-yl)pyrimidin-4-yl)-2-(4-methylpyrimidin-2-yl)cyclopropane-1-carboxamide;
(1S,2S)-2-(3-chlorophenyl)-N-(6-((2S,3S,4R)-2-(6-cyclopropylimidazo[1,2-a]pyridin-2-yl)-3,4-dihydroxypyrrolidin-1-yl)pyrimidin-4-yl)cyclopropane-1-carboxamide;
(1S,2S)-2-(3-chlorophenyl)-N-(6-((2R,3S)-2-(6-cyclopropylimidazo[1,2-a]pyridin-2-yl)-3-hydroxypyrrolidin-1-yl)pyrimidin-4-yl)cyclopropane-1-carboxamide;
(1S,2S)-2-(3-chlorophenyl)-N-(6-((2S,3R)-2-(6-cyclopropylimidazo[1,2-a]pyridin-2-yl)-3-hydroxypyrrolidin-1-yl)pyrimidin-4-yl)cyclopropane-1-carboxamide;
(1S,2S)—N-(6-((2R,4S)-2-(6-cyclopropylimidazo[1,2-a]pyridin-2-yl)-4-hydroxypyrrolidin-1-yl) pyrimidin-4-yl)-2-(3-ethynylphenyl)cyclopropane-1-carboxamide;
(1S,2S)-2-(4-chloropyridin-2-yl)-N-(6-(((6-cyclopropyl-8-(2,4-dioxoimidazolidin-1-yl)imidazo[1,2-a]pyridin-2-yl)methyl)amino)-2-hydroxypyrimidin-4-yl)cyclopropane-1-carboxamide;
(1S,2S)-2-(3-chlorophenyl)-N-(4-(((6-cyclopropyl-8-(4-hydroxy-1-methylpiperidin-4-yl)imidazo[1,2-a]pyridin-2-yl)methyl)amino)pyridin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(5-fluoro-4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)(methyl)amino)-2-methylpyrimidin-4-yl)-2-fluoro-2-(4-methylpyrimidin-2-yl)cyclopropane-1-carboxamide;
rac-(-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(5-fluoro-4-methylpyrimidin-2-yl)cyclopropane-1-carboxamide;
rac-(-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(5-fluoro-4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(2-methylthiazol-4-yl)cyclopropane-1-carboxamide;
rac-(1S*,3S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2,2-difluoro-3-(4-methylpyrimidin-2-yl)cyclopropane-1-carboxamide.
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(4-methyl-1,3,5-triazin-2-yl)cyclopropane-1-carboxamide;
rac-(1S*,2S*)—N-(6-(((6-cyclopropyl-8-(3-methyl-2,4-dioxoimidazolidin-1-yl)imidazo [1,2-a]pyridin-2-yl)methyl) amino)pyrimidin-4-yl)-2-fluoro-2-(4-methylpyrimidin-2-yl)cyclo propane-1-carboxamide;
rac-(1S*,2S*)—N-(6-(((6-cyclopropyl-8-(3-methyl-2,4-dioxoimidazolidin-1-yl).midazo [1,2-a]pyridin-2-yl)methyl) amino)pyrimidin-4-yl)-2-(4,6-dimethylpyrimidin-2-yl) cyclopropane-1-carboxamide;
rac-(1S*,2S*)—N-(6-(((6-cyclopropyl-8-(3-methyl-2,4-dioxoimidazolidin-1-yl).midazo[1,2-a]pyridin-2-yl) methyl)amino)pyrimidin-4-yl)-2-(6-methylpyrazin-2-yl)cyclo propane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(6-methylpyridin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)-2-(trifluoromethyl)pyrimidin-4-yl)-2-(4-methylpyrimidin-2-yl)cyclopropane-1-carboxamide;
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrimidin-4-yl)-2-(3-fluoro-6-methylpyridin-2-yl)cyclopropane-1-carboxamide;
rac-(1S*,3S*)-3-(3-chlorophenyl)-N-(6-(((6-cyclopropylimidazo[1,2-a]pyridin-2-yl)methyl)amino)pyrimidin-4-yl)-2,2-difluorocyclopropane-1-carboxamide;
rac-(1S*,2S*)-2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)cyclopropane-1-carboxamide;
(1S*,2S*,3R*)-2-(3-chlorophenyl)-N-(6-(((6-cyclopropylimidazo[1,2-a]pyridin-2-yl)methyl)amino)pyrimidin-4-yl)-3-methylcyclopropane-1-carboxamide;
rac-(1R*,2S*)-5′-chloro-N-(6-(((6-cyclopropylimidazo[1,2-a]pyridin-2-yl)methyl)amino)pyrimidin-4-yl)-2′-oxospiro[cyclopropane-1,3′-indoline]-2-carboxamide;
rac-(1R*,2R*)-5′-chloro-N-(6-(((6-cyclopropylimidazo[1,2-a]pyridin-2-yl)methyl)amino)pyrimidin-4-yl)-2′-oxospiro[cyclopropane-1,3′-indoline]-2-carboxamide;
rac-(1R*,2R*,3S*)-2-(3-chlorophenyl)-N-(6-(((6-cyclopropylimidazo[1,2-a]pyridin-2-yl)methyl)amino)pyrimidin-4-yl)-3-fluorocyclopropane-1-carboxamide;
(1S,2S)-2-(3-chlorophenyl)-N-(4-(((6-cyclopropyl-8-(2,4-dioxo-3-(2,2,2-trifluoroethyl)imidazolidin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-cyclopropyl-2,4-dioxoimidazolidin-1-yl)imidazo[1,2-a]pyridin-2-yl)methyl)amino)pyridin-2-yl)cyclopropane-1-carboxamide;
rac-(1R*,2S*)-5′-chloro-N-(4-(((6-cyclopropylimidazo[1,2-a]pyridin-2-yl)methyl)amino)pyridin-2-yl)-2′-oxospiro[cyclopropane-1,3′-indoline]-2-carboxamide;
rac-(1S*,2S*)—N-(4-(((8-(3-(2-(benzyloxy)ethyl)-2,4-dioxoimidazolidin-1-yl)-6-cyclopropylimidazo[1,2-a]pyridin-2-yl)methyl)amino)-5-fluoropyrimidin-2-yl)-2-(4-methylpyrimidin-2-yl)cyclopropane-1-carboxamide;
rac-(1S*,2S*)—N-(4-(((6-cyclopropyl-8-(2,4-dioxoimidazolidin-1-yl)imidazo[1,2-a]pyridin-2-yl)methyl)(methyl)amino)-5-fluoropyrimidin-2-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(trifluoromethyl)pyrimidin-2-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-methylpyrimidin-2-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide;
rac-(1S*,2S*)-2-(4-chloropyridin-2-yl)-N-(4-(((6-cyclopropyl-8-(2,4-dioxoimidazolidin-1-yl)imidazo[1,2-a]pyridin-2-yl)methyl)amino)pyrimidin-2-yl)cyclopropane-1-carboxamide;
rac-(1S*,2S*)—N-(4-cyclopropyl-6-(((6-cyclopropyl-8-(2,4-dioxoimidazolidin-1-yl)imidazo[1,2-a]pyridin-2-yl)methyl)(methyl)amino)-1,3,5-triazin-2-yl)-2-(4-methylpyrimidin-2-yl)cyclopropane-1-carboxamide;
rac-(1S*,2S*)—N-(4-cyclopropyl-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclopropane-1-carboxamide;
rac-(1S*,2S*)—N-(4-(((6-cyclopropyl-8-(2,4-dioxoimidazolidin-1-yl)imidazo[1,2-a]pyridin-2-yl)methyl)(methyl)amino)-6-methyl-1,3,5-triazin-2-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-methyl-1,3,5-triazin-2-yl)-2-(4-methylpyrimidin-2-yl)cyclopropane-1-carboxamide.
rac-(1S*,2S*)—N-(6-(((6-cyclopropyl-8-(3-methyl-2,4-dioxoimidazolidin-1-yl)imidazo[1,2-a]pyridin-2-yl)methyl)amino)pyrazin-2-yl)-2-(4-methylpyrimidin-2-yl)cyclopropane-1-carboxamide;
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-(5-methyl-1,2,4-thiadiazol-3-yl)cyclopropane-1-carboxamide;
rac-(1R*,2R*)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-1-fluoro-2-(4-methylpyrimidin-2-yl)cyclopropane-1-carboxamide;
rac-(1S*,2S*)—N-(6-(((R)-1-(6-cyclopropyl-8-(3-methyl-2,4-dioxoimidazolidin-1-yl)imidazo[1,2-a]pyridin-2-yl)ethyl)amino)pyrimidin-4-yl)-2-(4-methoxypyrimidin-2-yl)cyclopropane-1-carboxamide;
(1S,2S)—N-(6-(((R)-1-(6-cyclopropyl-8-(2,4-dioxoimidazolidin-1-yl)imidazo[1,2-a]pyridin-2-yl)ethyl)amino)-2-methylpyrimidin-4-yl)-2-(4-methylpyrimidin-2-yl)cyclopropane-1-carboxamide;
(1S,2S)—N-(6-(((R)-1-(6-cyclopropyl-8-(2,4-dioxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((R)-1-(6-cyclopropyl-8-(2-oxooxazolidin-3-yl)imidazo[1,2-a]pyridin-2-yl)ethyl)amino)pyrimidin-4-yl)-2-(4-methylpyrimidin-2-yl)cyclopropane-1-carboxamide;
(1S,2S)—N-(6-(((6-(2,2-difluorocyclopropyl)-8-(3-methyl-2,4-dioxoimidazolidin-1-yl)imidazo[1,2-a]pyridin-2-yl)methyl)amino)pyrimidin-4-yl)-2-(4-methylpyrimidin-2-yl)cyclopropane-1-carboxamide;
(1S,2S)—N-(6-(((R)-1-(6-cyclopropyl-8-(3-methyl-2-oxoimidazolidin-1-yl)imidazo[1,2-a]pyridin-2-yl)ethyl)amino)pyrimidin-4-yl)-2-(4-methylpyrimidin-2-yl)cyclopropane-1-carboxamide;
rac-(1S*,2S*)—N-(4-cyclopropyl-6-(((6-cyclopropyl-8-(3-methyl-2,4-dioxoimidazolidin-1-yl)imidazo[1,2-a]pyridin-2-yl)methyl)amino)pyrimidin-2-yl)-2-(4-methylpyrimidin-2-yl)cyclopropane-1-carboxamide;
rac-(1S*,2S*)—N-(4-(((6-cyclopropyl-8-(3-(2-hydroxyethyl)-2,4-dioxoimidazolidin-1-yl)imidazo[1,2-a]pyridin-2-yl)methyl)amino)-5-fluoropyrimidin-2-yl)-2-(4-methylpyrimidin-2-yl)cyclopropane-1-carboxamide;
rac-(1S*,2S*)—N-(4-((2R,4S)-2-(6-cyclopropyl-8-(3-methyl-2,4-dioxoimidazolidin-1-yl)imidazo[1,2-a]pyridin-2-yl)-4-hydroxypyrrolidin-1-yl)pyrimidin-2-yl)-2-(4-methylpyrimidin-2-yl)cyclopropane-1-carboxamide;
rac-(1S*,2S*)—N-(6-((2R,4S)-2-(6-cyclopropylimidazo[1,2-a]pyridin-2-yl)-4-hydroxypyrrolidin-1-yl)pyrimidin-4-yl)-2-(3-fluoro-4-methylpyridin-2-yl)cyclopropane-1-carboxamide;
rac-(1S*,3S*)-3-(3-chlorophenyl)-N-(6-(((6-cyclopropyl-8-(3-methyl-2,4-dioxo.midazolidin-1-yl)imidazo[1,2-a]pyridin-2-yl)methyl)amino)pyrimidin-4-yl)-2,2-difluorocyclopropane-1-carboxamide;
(1S,3S)-3-(3-chlorophenyl)-N-(6-(((6-cyclopropyl-8-(3-methyl-2,4-dioxo.midazolidin-1-yl)imidazo[1,2-a]pyridin-2-yl)methyl)amino)pyrimidin-4-yl)-2,2-difluorocyclopropane-1-carboxamide;
(1R,3R)-3-(3-chlorophenyl)-N-(6-(((6-cyclopropyl-8-(3-methyl-2,4-dioxo.midazolidin-1-yl)imidazo[1,2-a]pyridin-2-yl)methyl)amino)pyrimidin-4-yl)-2,2-difluorocyclopropane-1-carboxamide;
(1S,2S)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide;
(1R,2R)—N-(6-(((R)-1-(6-cyclopropyl-8-(3-methyl-2,4-dioxoimidazolidin-1-yl)imidazo[1,2-a]pyridin-2-yl)ethyl)amino)-2-methylpyrimidin-4-yl)-2-fluoro-2-(4-methylpyrimidin-2-yl)cyclopropane-1-carboxamide;
(1S,2S)—N-(6-(1-(6-cyclopropyl-8-(3-methyl-2,4-dioxoimidazolidin-1-yl)imidazo[1,2-a]pyridin-2-yl)ethoxy)pyrimidin-4-yl)-2-(4-methylpyrimidin-2-yl)cyclopropane-1-carboxamide;
methyl 2-(3-(6-cyclopropyl-2-(((2-methyl-6-((1S,2S)-2-(4-methylpyrimidin-2-yl)cyclopropane-1-carboxamido)pyrimidin-4-yl)oxy)methyl)imidazo[1,2-a]pyridin-8-yl)-2,5-dioxoimidazolidin-1-yl)acetate;
(1S,2S)—N-(6-((6-cyclopropyl-8-(3-methyl-2,4-dioxoimidazolidin-1-yl)imidazo[1,2-a]pyridin-2-yl)methoxy)-2-(methoxymethyl)pyrimidin-4-yl)-2-(4-methylpyrimidin-2-yl)cyclopropane-1-carboxamide;
(1S,2S)—N-(6-(1-(6-cyclopropyl-8-(3-methyl-2,4-dioxoimidazolidin-1-yl)imidazo[1,2-a]pyridin-2-yl)ethoxy)-2-methylpyrimidin-4-yl)-2-(4-methylpyrimidin-2-yl)cyclopropane-1-carboxamide;
(1S,2S)—N-(6-((6-cyclopropyl-8-(6-oxo-2,5-dioxa-7-azaspiro[3.4]octan-7-yl)imidazo[1,2-a]pyridin-2-yl) methoxy)-2-methylpyrimidin-4-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-2-methoxypyrimidin-4-yl)-2-(4-methylpyrimidin-2-yl)cyclopropane-1-carboxamide;
(1S,2S)-2-(3-chlorophenyl)-N-(5-((6-cyclopropyl-8-(3-methyl-2,4-dioxoimidazolidin-1-yl)imidazo[1,2-a]pyridin-2-yl)methoxy)pyridazin-3-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide;
rac-(1S*,2S*)-2-(2-cyano-5-methoxyphenyl)-N-(5-((6-cyclopropyl-8-(3-methyl-2,4-dioxoimidazolidin-1-yl)imidazo[1,2-a]pyridin-2-yl)methoxy)pyridazin-3-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)-6-methylpyridazin-3-yl)-2-(4-methylpyrimidin-2-yl)cyclopropane-1-carboxamide;
(1S,2S)—N-(5-((6-cyclopropyl-8-(methylsulfonyl)imidazo[1,2-a]pyridin-2-yl)methoxy)pyridazin-3-yl)-2-(4-methylpyrimidin-2-yl)cyclopropane-1-carboxamide;
(1S,2S)—N-(6-((6-cyclopropyl-8-(3-(2-hydroxy-2-methylpropyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-2-(2-(dimethylamino)ethoxy)pyrimidin-4-yl)-2-(4-methylpyrimidin-2-yl)cyclopropane-1-carboxamide;
(1S,2S)—N-(6-((6-cyclopropyl-8-(3-(2-(dimethylamino)ethyl)-2,4-dioxoimidazolidin-1-yl)imidazo[1,2-a]pyridin-2-yl)methoxy)-2-methylpyrimidin-4-yl)-2-(4-methylpyrimidin-2-yl)cyclopropane-1-carboxamide;
(1S,2S)-2-(3-chlorophenyl)-N-(6-(((5-cyclopropyl-7-(4-methylpiperazin-1-yl)pyrazolo[1,5-a]pyridin-2-yl)methyl)amino)pyrimidin-4-yl)cyclopropane-1-carboxamide;
rac-(1S*,2S*)-2-(3-chlorophenyl)-N-(4-((8-(2-cyanoethyl)-6-cyclopropylimidazo[1,2-a]pyridin-2-yl)methoxy)pyridin-2-yl)cyclopropane-1-carboxamide;
rac-(1S*,2S*)-2-(3-chlorophenyl)-N-(4-((8-cyano-6-cyclopropylimidazo[1,2-a]pyridin-2-yl)methoxy)pyridin-2-yl)cyclopropane-1-carboxamide;
(1S,2S)—N-(5-((6-cyclopropylimidazo[1,2-a]pyridin-2-yl)methoxy)pyridazin-3-yl)-2-(4-methyl pyrimidin-2-yl)cyclopropane-1-carboxamide;
(1S,2S)-2-(3-chlorophenyl)-N-(3-((6-cyclopropylimidazo[1,2-a]pyridin-2-yl)methoxy)pyridin-2-yl)cyclopropane-1-carboxamide;
(1S,2S)-2-(3-chlorophenyl)-N-(3-((6-cyclopropylimidazo[1,2-a]pyridin-2-yl)methoxy)pyrazin-2-yl)cyclopropane-1-carboxamide;
(1S,2S)—N-(5-((6-cyclopropyl-8-(2-oxo-3-azabicyclo[3.1.0]hexan-3-yl)imidazo[1,2-a]pyridin-2-yl)methoxy)pyridazin-3-yl)-2-(4-methylpyrimidin-2-yl)cyclopropane-1-carboxamide;
rac-(1S*,2S*)—N-(4-((8-(aminomethyl)-6-cyclopropylimidazo[1,2-a]pyridin-2-yl)methoxy)pyridin-2-yl)-2-(3-chlorophenyl)cyclopropane-1-carboxamide;
(1S,2S)—N-(4-((6-cyclopropyl-8-(3-methyl-2,4-dioxoimidazolidin-1-yl)imidazo[1,2-a]pyridin-2-yl)methoxy)-6-methyl-1,3,5-triazin-2-yl)-2-(4-methylpyrimidin-2-yl)cyclopropane-1-carboxamide
rac-(1S*,2S*)-2-(3-chlorophenyl)-N-(5-(((6-cyclopropylimidazo[1,2-a]pyridin-2-yl)methyl)amino)pyridin-3-yl)cyclopropane-1-carboxamide;
rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridin-3-yl)-2-(4-methylpyrimidin-2-yl)cyclopropane-1-carboxamide;
rac-(1S*,2S*)—N-(2-((6-cyclopropyl-8-(3-methyl-2,4-dioxoimidazolidin-1-yl)imidazo[1,2-a]pyridin-2-yl)methyl)-2H-pyrazolo[4,3-c]pyridin-4-yl)-2-(4-methylpyrimidin-2-yl)cyclopropane-1-carboxamide;
(1S,2S)-2-(4-chloropyridin-2-yl)-N-(2-((6-cyclopropylimidazo[1,2-a]pyridin-2-yl)methyl)-2H-pyrazolo[4,3-c]pyridin-4-yl)cyclopropane-1-carboxamide;
rac-(1S*,2S*)-2-(5-chloro-2-nitrophenyl)-N-(2-((6-cyclopropylimidazo[1,2-a]pyridin-2-yl)methyl)-2H-pyrazolo[4,3-c]pyridin-4-yl)cyclopropane-1-carboxamide.
rac-(1S*,2S*)-2-(5-chloro-2-cyanophenyl)-N-(2-((6-cyclopropylimidazo[1,2-a]pyridin-2-yl)methyl)-2H-pyrazolo[4,3-c]pyridin-4-yl)cyclopropane-1-carboxamide;
(1S,2S)-2-(3-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide;
(1S,2S)-2-(3-chlorophenyl)-N-(2-((6-cyclopropylimidazo[1,2-a]pyridin-2-yl)methyl)-6-methyl-2H-pyrazolo[4,3-c]pyridin-4-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide;
(1S,2S)-2-(3-chlorophenyl)-N-(2-((6-cyclopropylimidazo[1,2-a]pyridin-2-yl)methyl)-2H-[1,2,3]triazolo[4,5-c]pyridin-4-yl)cyclopropane-1-carboxamide;
(1S,2S)-2-(3-chlorophenyl)-N-(1-((6-cyclopropylimidazo[1,2-a]pyridin-2-yl)methyl)-1H-pyrazolo[4,3-c]pyridin-4-yl)cyclopropane-1-carboxamide;
rac-(1S*,2S*)-2-(2-amino-5-chlorophenyl)-N-(2-((6-cyclopropylimidazo[1,2-a]pyridin-2-yl)methyl)-2H-pyrazolo[4,3-c]pyridin-4-yl)cyclopropane-1-carboxamide;
rac-(1S*,2S*)-2-(5-chloro-2-(1H-tetrazol-1-yl)phenyl)-N-(2-((6-cyclopropylimidazo[1,2-a]pyridin-2-yl)methyl)-2H-pyrazolo[4,3-c]pyridin-4-yl)cyclopropane-1-carboxamide;
rac-(1S*,2S*)-2-(3-chlorophenyl)-N-(6-((6-cyclopropylimidazo[1,2-a]pyridin-2-yl)methyl)pyrimidin-4-yl)cyclopropane-1-carboxamide;
rac-(1S*,2S*)-2-(3-chlorophenyl)-N-((1-((6-cyclopropylimidazo[1,2-a]pyridin-2-yl)methyl)-1H-1,2,3-triazol-4-yl)methyl)cyclopropane-1-carboxamide;
rac-(1S*,2S*)-2-(3-chlorophenyl)-N-(1-(1-((6-cyclopropylimidazo[1,2-a]pyridin-2-yl)methyl)-1H-1,2,3-triazol-4-yl)ethyl)cyclopropane-1-carboxamide;
(1S,2S)-2-(3-chlorophenyl)-N-((1-((6-cyclopropyl-8-(4H-1,2,4-triazol-4-yl)imidazo[1,2-a]pyridin-2-yl)methyl)-1H-1,2,3-triazol-4-yl)methyl)cyclopropane-1-carboxamide;
rac-(1S*,2S*)-2-(3-chlorophenyl)-N-(2-((6-cyclopropylimidazo[1,2-a]pyridin-2-yl)methyl)-2H-pyrazolo[3,4-c]pyridin-7-yl)cyclopropane-1-carboxamide;
rac-(1S*,2S*)-2-(3-chlorophenyl)-N-(1-((6-cyclopropylimidazo[1,2-a]pyridin-2-yl)methyl)-1H-1,2,3-triazol-4-yl)cyclopropane-1-carboxamide;
rac-(1S*,2S*)-2-(3-chlorophenyl)-N-(1-((6-cyclopropylimidazo[1,2-a]pyridin-2-yl)methyl)-1H-imidazo[4,5-c]pyridin-6-yl)cyclopropane-1-carboxamide;
(1S,2S)-2-(3-chlorophenyl)-N-(4-(((6-cyclopropyl-8-((methylamino)methyl)imidazo[1,2-a]pyridin-2-yl)methyl)amino)pyridin-2-yl)cyclopropane-1-carboxamide;
(1S,2S)-2-(3-chlorophenyl)-N-(5-((6-cyclopropylimidazo[1,2-a]pyridin-2-yl)methoxy)pyrimidin-4-yl)cyclopropane-1-carboxamide;
(1S,2S)-2-(3-chlorophenyl)-N-(1-((6-cyclopropylimidazo[1,2-a]pyridin-2-yl)methyl)-2,3-dihydro-1H-pyrido[3,4-b][1,4]oxazin-7-yl)cyclopropane-1-carboxamide;
(1S,2S)-2-(3-chlorophenyl)-N-(4-(((6-cyclopropyl-8-(2-(dimethylamino)ethyl)imidazo [1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(4-(2-methoxyethyl)piperazin-1-yl)imidazo[1,2-a]pyridin-2-yl)methyl)amino)pyridin-2-yl)cyclopropane-1-carboxamide;
ethyl 2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetate;
(1S,2S)-2-(3-chlorophenyl)-N-(4-(((6-cyclopropyl-8-(4-(cyclopropylmethyl)piperazin-1-yl)imidazo[1,2-a]pyridin-2-yl)methyl)amino)pyridin-2-yl)cyclopropane-1-carboxamide;
2-(4-(2-(((2-((1S,2S)-2-(3-chlorophenyl)cyclopropane-1-carboxamido)pyridin-4-yl)amino)methyl)-6-cyclopropylimidazo[1,2-a]pyridin-8-yl)piperazin-1-yl)acetic acid;
(1S,2S)-2-(3-chlorophenyl)-N-(4-(((6-cyclopropyl-8-(4-(2-hydroxyethyl)piperazin-1-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropylimidazo[1,2-a]pyridin-2-yl)methyl)(2-hydroxyethyl)amino)pyridin-2-yl)cyclopropane-1-carboxamide;
(1S,2S)-2-(3-chlorophenyl)-N-(4-(((6-cyclopropyl-8-(4-methylpiperazin-1-yl)imidazo[1,2-a]pyridin-2-yl)methyl)amino)-6-methylpyridin-2-yl)cyclopropane-1-carboxamide;
(1S,2S)-2-(3-chlorophenyl)-N-(5-(((6-cyclopropylimidazo[1,2-a]pyridin-2-yl)methyl)amino)-1,3,4-thiadiazol-2-yl)cyclopropane-1-carboxamide;
(1S,2S)-2-(3-chlorophenyl)-N-(4-(((6-cyclopropyl-8-(2-(methylamino)ethyl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(3-hydroxy-1-methylazetidin-3-yl)imidazo[1,2-a]pyridin-2-yl)methyl)amino)pyridin-2-yl)cyclopropane-1-carboxamide;
(1S,2S)-2-(3-chlorophenyl)-N-(4-(((6-cyclopropyl-8-(1-methylazetidin-3-yl)imidazo[1,2-a]pyridin-2-yl)methyl)amino)pyridin-2-yl)cyclopropane-1-carboxamide;
(1S,2S)-2-(3-chlorophenyl)-N-(5-(((6-cyclopropylimidazo[1,2-a]pyridin-2-yl)methyl)amino)-1,2,4-thiadiazol-3-yl)cyclopropane-1-carboxamide;
(1S,2S)-2-(3-chlorophenyl)-N-(4-(((6-cyclopropylimidazo[1,2-a]pyridin-2-yl)methyl)amino)-6-hydroxypyridin-2-yl)cyclopropane-1-carboxamide;
(1S,2S)-2-(3-chlorophenyl)-N-(5-(((6-cyclopropyl-8-(4-methylpiperazin-1-yl)imidazo[1,2-a]pyridin-2-yl)methyl)amino)pyridazin-3-yl)cyclopropane-1-carboxamide;
(1S,2S)-2-(3-chlorophenyl)-N-(2-(((6-cyclopropyl-8-(4-methylpiperazin-1-yl)imidazo[1,2-a]pyridin-2-yl)methyl)amino)pyrimidin-4-yl)cyclopropane-1-carboxamide;
rac-(1S*,2S*)-2-(3-chloropyridinyl)-N-(5-(((6-cyclopropylimidazo[1,2-a]pyridin-2-yl)methyl)amino)-1,2,4-thiadiazol-3-yl)cyclopropane-1-carboxamide;
(1S,2S)-2-(3-chlorophenyl)-N-(5-(((6-cyclopropylimidazo[1,2-a]pyridin-2-yl)methyl)amino)-1,2,4-oxadiazol-3-yl)cyclopropane-1-carboxamide;
(1S,2S)-2-(3-chlorophenyl)-N-(6-((2R,4S)-2-(6-cyclopropylimidazo[1,2-a]pyridin-2-yl)-4-methoxypyrrolidin-1-yl)pyrimidin-4-yl)cyclopropane-1-carboxamide;
rac-(1S*,2S*)—N-(5-(((6-cyclopropyl-8-(3-methyl-2,4-dioxoimidazolidin-1-yl)imidazo[1,2-a]pyridin-2-yl)methyl)amino)pyridazin-3-yl)-2-(4-methylpyrimidin-2-yl)cyclo propane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-6-(trifluoromethyl)pyridin-2-yl)-2-(4-methylpyrimidin-2-yl)cyclopropane-1-carboxamide;
rac-(1S*,2S*)—N-(5-(((6-cyclopropylimidazo[1,2-a]pyridin-2-yl)methyl)amino)-2-(methylsulfonyl)phenyl)-2-(4-methylpyrimidin-2-yl)cyclopropane-1-carboxamide;
rac-(1S*,2S*)—N-(6-((6-cyclopropyl-8-(3-methyl-2,4-dioxoimidazolidin-1-yl)imidazo [1,2-a]pyridin-2-yl)methyl)pyrimidin-4-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(methoxymethyl)pyrimidin-2-yl)-2-(4-methylpyrimidin-2-yl)cyclopropane-1-carboxamide;
rac-(1S*,2S*)—N-(4-chloro-6-(((6-cyclopropyl-8-(3-methyl-2,4-dioxoimidazolidin-1-yl)imidazo[1,2-a]pyridin-2-yl)methyl)amino)-1,3,5-triazin-2-yl)-2-(4-methylpyrimidin-2-yl)cyclo propane-1-carboxamide;
rac-(1S*,2S*)—N-(4-(((6-cyclopropyl-8-(3-methyl-2,4-dioxo.midazolidin-1-yl)imidazo[1,2-a]pyridin-2-yl)methyl)amino)-1,3,5-triazin-2-yl)-2-(4-methylpyrimidin-2-yl)cyclopropane-1-carboxamide;
rac-(1S*,2S*)—N-(7-(((6-cyclopropyl-8-(3-methyl-2,4-dioxoimidazolidin-1-yl)imidazo[1,2-a]pyridin-2-yl)methyl)amino)-[1,2,4]triazolo[1,5-a]pyrimidin-5-yl)-2-(4-methylpyrimidin-2-yl)cyclopropane-1-carboxamide;
rac-(1S*,2S*)—N-(6-(2-(6-cyclopropyl-8-(3-methyl-2,4-dioxoimidazolidin-1-yl)imidazo[1,2-a]pyridin-2-yl)ethyl)pyrimidin-4-yl)-2-(4-methylpyrimidin-2-yl)cyclopropane-1-carboxamide;
rac-(1S*,2S*)—N-(3-((6-cyclopropyl-8-(3-methyl-2,4-dioxoimidazolidin-1-yl)imidazo[1,2-a]pyridin-2-yl)methyl)-2-oxo-2,3-dihydrooxazolo[4,5-d]pyrimidin-5-yl)-2-(4-methylpyrimidin-2-yl)cyclopropane-1-carboxamide;
rac-(1S*,2S*)—N-(4-(((6-cyclopropyl-8-(3-methyl-2,4-dioxoimidazolidin-1-yl)imidazo[1,2-a]pyridin-2-yl)methyl)amino)-5-(hydroxymethyl)pyrimidin-2-yl)-2-(4-methylpyrimidin-2-yl)cyclopropane-1-carboxamide;
rac-(1S*,2S*)—N-(4-((6-cyclopropyl-8-(3-methyl-2,4-dioxoimidazolidin-1-yl)imidazo[1,2-a]pyridin-2-yl)methoxy)-5-(trifluoromethyl)pyrimidin-2-yl)-2-(4-methylpyrimidin-2-yl)cyclopropane-1-carboxamide;
(1S,2S)—N-(2-((6-cyclopropyl-8-(3-methyl-2,4-dioxoimidazolidin-1-yl)imidazo[1,2-a]pyridin-2-yl)methyl)oxazolo[5,4-c]pyridin-4-yl)-2-(4-methylpyrimidin-2-yl)cyclopropane-1-carboxamide;
(1S,2S)—N-(3-((6-cyclopropyl-8-(3-methyl-2,4-dioxoimidazolidin-1-yl)imidazo[1,2-a]pyridin-2-yl)methyl)-[1,2,4]triazolo[4,3-a]pyrazin-6-yl)-2-(4-methylpyrimidin-2-yl)cyclopropane-1-carboxamide;
(1S,2S)-2-(4-chloropyrimidin-2-yl)-N-(8-((6-cyclopropyl-8-(3-methyl-2,4-dioxoimidazolidin-1-yl)imidazo[1,2-a]pyridin-2-yl)methyl)-7,8-dihydro-6H-pyrimido[5,4-b][1,4]oxazin-2-yl)cyclopropane-1-carboxamide;
rac-(1S*,2S*)—N-(2-((6-cyclopropyl-8-(3-methyl-2,4-dioxoimidazolidin-1-yl)imidazo [1,2-a]pyridin-2-yl)methyl)-2H-pyrazolo[3,4-d]pyrimidin-4-yl)-2-(4-methylpyrimidin-2-yl)cyclopropane-1-carboxamide;
(1S,2S)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide;
(1R,2R)—N-(2-((6-cyclopropyl-8-(3-methyl-2,4-dioxoimidazolidin-1-yl)imidazo[1,2-a]pyridin-2-yl)methyl)-2H-pyrazolo[3,4-d]pyrimidin-4-yl)-2-(4-methylpyrimidin-2-yl)cyclopropane-1-carboxamide;
(1S,2S)—N-(1-((6-cyclopropyl-8-(3-methyl-2,4-dioxoimidazolidin-1-yl)imidazo[1,2-a]pyridin-2-yl)methyl)-1H-[1,2,3]triazolo[4,5-c]pyridin-6-yl)-2-(4-methylpyrimidin-2-yl)cyclopropane-1-carboxamide;
(1S,2S)—N-(4-(((8-(3-(2-(benzyloxy)ethyl)-2,4-dioxoimidazolidin-1-yl)-6-cyclopropylimidazo[1,2-a]pyridin-2-yl)methyl)(methyl)amino)-6-cyclopropylpyridin-2-yl)-2-(4-methylpyrimidin-2-yl)cyclopropane-1-carboxamide;
(1S,2S)—N-(6-cyclopropyl-4-(((6-cyclopropyl-8-(3-(2-hydroxyethyl)-2,4-dioxoimidazolidin-1-yl) imidazo[1,2-a]pyridin-2-yl)methyl)(methyl)amino)pyridin-2-yl)-2-(4-methylpyrimidin-2-yl) cyclopropane-1-carboxamide;
(1S,2S)—N-(6-(((6-cyclopropyl-8-(3-methyl-2,4-dioxoimidazolidin-1-yl)imidazo[1,2-a]pyridin-2-yl)methyl)thio)-2-methylpyrimidin-4-yl)-2-(4-methylpyrimidin-2-yl)cyclopropane-1-carboxamide;
(1S,2S)—N-(2-(((6-cyclopropyl-8-(3-methyl-2,4-dioxoimidazolidin-1-yl)imidazo[1,2-a]pyridin-2-yl)methyl)amino)-6-methylpyridin-4-yl)-2-(4-methylpyrimidin-2-yl)cyclopropane-1-carboxamide;
(1S,2S)—N-(5-(((6-cyclopropyl-8-(3-methyl-2,4-dioxoimidazolidin-1-yl)imidazo[1,2-a]pyridin-2-yl)methyl)amino)-6-(trifluoromethyl)pyridazin-3-yl)-2-(4-methylpyrimidin-2-yl)cyclopropane-1-carboxamide;
(1S,2S)-2-(3-chlorophenyl)-N-(5-(((6-cyclopropyl-8-(3-methyl-2,4-dioxoimidazolidin-1-yl)imidazo[1,2-a]pyridin-2-yl)methyl)(methyl)amino)pyridazin-3-yl)cyclopropane-1-carboxamide;
(1S,2S)—N-(4-(((6-cyclopropyl-8-(3-methyl-2,4-dioxoimidazolidin-1-yl)imidazo-[1,2-a]pyridin-2-yl)methyl)amino)-6-methylpyridin-2-yl)-2-(4-methylpyrimidin-2-yl)cyclopropane-1-carboxamide;
(1S,2S)—N-(3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide;
(1S,2S)—N-(3-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide;
(1S,2S)—N-(3-(((6-cyclopropyl-8-(3-methyl-2,4-dioxoimidazolidin-1-yl)imidazo[1,2-a]pyridin-2-yl)methyl)thio)phenyl)-2-(4-methylpyrimidin-2-yl)cyclopropane-1-carboxamide;
(1S,2S)—N-(3-(((6-cyclopropyl-8-(3-methyl-2,4-dioxoimidazolidin-1-yl)imidazo[1,2-a]pyridin-2-yl)methyl)sulfonyl)phenyl)-2-(4-methylpyrimidin-2-yl)cyclopropane-1-carboxamide;
(1S,2S)-2-(3-chlorophenyl)-N-(6-(((6-cyclopropyl-8-(2-oxopiperazin-1-yl)imidazo[1,2-a]pyridin-2-yl)methyl)amino)pyrimidin-4-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)pyridazin-4-yl)-2-(4-methylpyrimidin-2-yl)cyclopropane-1-carboxamide;
(1S,2S)—N-(2-((6-cyclopropyl-8-(3-methyl-2,4-dioxoimidazolidin-1-yl)imidazo[1,2-a]pyridin-2-yl)methyl)pyrazolo[1,5-a]pyrazin-4-yl)-2-(4-methylpyrimidin-2-yl)cyclopropane-1-carboxamide;
(1S,2S)—N-(6-((1-(6-cyclopropyl-8-(3-methyl-2,4-dioxoimidazolidin-1-yl)imidazo[1,2-a]pyridin-2-yl)-2-hydroxyethyl)amino)-2-methylpyrimidin-4-yl)-2-(4-methylpyrimidin-2-yl)cyclopropane-1-carboxamide;
(1S,2S)—N-(5-(((6-cyclopropyl-8-(3-methyl-2,4-dioxoimidazolidin-1-yl)imidazo[1,2-a]pyridin-2-yl)methyl)amino)-1,2,4-triazin-3-yl)-2-(4-methylpyrimidin-2-yl)cyclopropane-1-carboxamide;
(1S,2S)—N-(2-((6-cyclopropyl-8-(3-methyl-2,4-dioxoimidazolidin-1-yl)imidazo[1,2-a]pyridin-2-yl)methoxy)-6-methylpyrimidin-4-yl)-2-(4-methylpyrimidin-2-yl)cyclopropane-1-carboxamide;
(1S,2S)—N-(4-(2-(6-cyclopropyl-8-(3-methyl-2,4-dioxoimidazolidin-1-yl)imidazo[1,2-a]pyridin-2-yl)ethyl)pyridin-2-yl)-2-(4-methylpyrimidin-2-yl)cyclopropane-1-carboxamide;
ethyl 4-((6-cyclopropyl-8-(3-methyl-2,4-dioxoimidazolidin-1-yl).midazo[1,2-a]pyridin-2-yl)methoxy)-2-((1S,2S)-2-(4-methylpyrimidin-2-yl)cyclopropane-1-carboxamido)pyrimidine-5-carboxylate;
(1S,2S)—N-(3-cyano-5-(((6-cyclopropyl-8-(3-methyl-2,4-dioxoimidazolidin-1-yl)imidazo[1,2-a]pyridin-2-yl)methyl)amino)phenyl)-2-(4-methylpyrimidin-2-yl)cyclopropane-1-carboxamide;
(1S,2S)—N-(3-(((6-cyclopropyl-8-(3-methyl-2,4-dioxoimidazolidin-1-yl)imidazo[1,2-a]pyridin-2-yl)methyl)amino)-4-fluorophenyl)-2-(4-methylpyrimidin-2-yl)cyclopropane-1-carboxamide;
(1S,2S)—N-(3-((6-cyclopropyl-8-(3-methyl-2,4-dioxoimidazolidin-1-yl)imidazo[1,2-a]pyridin-2-yl)methoxy)-4-fluorophenyl)-2-(4-methylpyrimidin-2-yl)cyclopropane-1-carboxamide;
(1S,2S)—N-(4-chloro-3-(((6-cyclopropyl-8-(3-methyl-2,4-dioxoimidazolidin-1-yl)imidazo[1,2-a]pyridin-2-yl)methyl)amino)-5-fluorophenyl)-2-(4-methylpyrimidin-2-yl)cyclopropane-1-carboxamide;
(1S,2S)—N-(5-(((6-cyclopropyl-8-(3-methyl-2,4-dioxoimidazolidin-1-yl)imidazo[1,2-a]pyridin-2-yl)methyl)amino)-2,4-difluorophenyl)-2-(4-methylpyrimidin-2-yl)cyclopropane-1-carboxamide;
(1S,2S)—N-(4-(benzyloxy)-6-((6-cyclopropyl-8-(3-methyl-2,4-dioxoimidazolidin-1-yl)imidazo[1,2-a]pyridin-2-yl)methoxy)pyridin-2-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-4-hydroxypyridin-2-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-2-(1-hydroxyethyl)pyrimidin-4-yl)-2-(4-methylpyrimidin-2-yl)cyclopropane-1-carboxamide;
(1S,2S)—N-(4-((6-cyclopropyl-8-(3-methyl-2,4-dioxoimidazolidin-1-yl)imidazo[1,2-a]pyridin-2-yl)methoxy)-5-(methoxymethyl)pyrimidin-2-yl)-2-(4-methylpyrimidin-2-yl)cyclopropane-1-carboxamide;
(1S,2S)—N-(4-((6-cyclopropyl-8-(3-methyl-2,4-dioxoimidazolidin-1-yl)imidazo[1,2-a]pyridin-2-yl)methoxy)-5-(hydroxymethyl)pyrimidin-2-yl)-2-(4-methylpyrimidin-2-yl)cyclopropane-1-carboxamide;
(1S,2S)—N-(5-(((R)-1-(6-cyclopropyl-8-(3-methyl-2,4-dioxoimidazolidin-1-yl).midazo[1,2-a]pyridin-2-yl)ethyl)amino)-1,2,4-triazin-3-yl)-2-(4-methylpyrimidin-2-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)-6-(trifluoromethyl)pyridazin-3-yl)-2-(4-methylpyrimidin-2-yl)cyclopropanecarboxamide;
(1S,2S)—N-(5-(2-(6-cyclopropyl-8-(3-methyl-2,4-dioxoimidazolidin-1-yl)imidazo[1,2-a]pyridin-2-yl)ethyl)pyridazin-3-yl)-2-(4-methylpyrimidin-2-yl)cyclopropane-1-carboxamide;
(1S,2S)—N-(5-(((6-cyclopropyl-8-(3-methyl-2,4-dioxoimidazolidin-1-yl)imidazo[1,2-a]pyridin-2-yl)methyl)(methyl)amino)-1,2,4-triazin-3-yl)-2-(4-methylpyrimidin-2-yl)cyclopropane-1-carboxamide;
(1S,2S)—N-(4-cyano-3-((6-cyclopropyl-8-(3-methyl-2,4-dioxoimidazolidin-1-yl)imidazo[1,2-a]pyridin-2-yl)methoxy)phenyl)-2-(4-methylpyrimidin-2-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)-6-(methylamino)pyridazin-3-yl)-2-(4-methylpyrimidin-2-yl)cyclopropane-1-carboxamide;
(1S,2S)—N-(5-((6-cyclopropyl-8-(3-methyl-2,4-dioxoimidazolidin-1-yl)imidazo[1,2-a]pyridin-2-yl)methoxy)-1,2,4-triazin-3-yl)-2-(4-methylpyrimidin-2-yl)cyclopropane-1-carboxamide;
(1S,2S)—N-(5-(1-(6-cyclopropyl-8-(3-methyl-2,4-dioxoimidazolidin-1-yl)imidazo[1,2-a]pyridin-2-yl)ethoxy)-1,2,4-triazin-3-yl)-2-(4-methylpyrimidin-2-yl)cyclopropane-1-carboxamide;
2-(6-cyclopropyl-8-(3-methyl-2,4-dioxoimidazolidin-1-yl)imidazo[1,2-a]pyridin-2-yl)-1-(2-methyl-6-((1S,2S)-2-(4-methylpyrimidin-2-yl)cyclopropane-1-carboxamido)pyrimidin-4-yl)ethyl acetate;
(1S,2S)—N-(6-(2-(6-cyclopropyl-8-(3-methyl-2,4-dioxoimidazolidin-1-yl)imidazo[1,2-a]pyridin-2-yl)-1-hydroxyethyl)-2-methylpyrimidin-4-yl)-2-(4-methylpyrimidin-2-yl)cyclopropane-1-carboxamide;
(1S,2S)—N-(6-((6-cyclopropyl-8-(3-methyl-2,4-dioxoimidazolidin-1-yl)imidazo[1,2-a]pyridin-2-yl)methoxy)-4-fluoropyridin-2-yl)-2-(4-methylpyrimidin-2-yl)cyclopropane-1-carboxamide;
(1S,2S)—N-(5-((6-(1-fluorocyclopropyl)-8-(3-methyl-2,4-dioxoimidazolidin-1-yl)imidazo[1,2-a]pyridin-2-yl)methoxy)pyridazin-3-yl)-2-(4-methylpyrimidin-2-yl)cyclopropane-1-carboxamide; and
(1S,2S)—N-(5-((6-cyclopropyl-8-(3-methyl-2,4-dioxoimidazolidin-1-yl)imidazo[1,2-a]pyridin-2-yl)methoxy)-6-(hydroxymethyl)pyridazin-3-yl)-2-(4-methylpyrimidin-2-yl)cyclopropane-1-carboxamide.

32. A pharmaceutical composition comprising a compound of claim 1.

33. A method of treating a plasma kallikrein-mediated disease or disorder using a compound of claim 1.

34. The method of claim 33, wherein the disease or disorder is hereditary angioedema or diabetic macular edema.
